SEQUENCE LISTING (101 sequences)

SEQ ID NO: 1 - DNA, 395 bp, Homo sapiens
  misc_feature (1)...(395): n = A,T,C or G
ctacttagggcgaattggagctccccgcggtggcggccgaggtacgcggggagagtgttccagacagaagaaatagcaagtgccgagaagctggcatcagaaaaacagaggggagatttgtgtggctgcagccgagggagaccaggaagatctgcatggtgggaaggacctgatgatacagaggtgagaaataagaaaggctgctgactttaccatctgaggccacacatctgctgaaatggagataattaacatcactagaaacagcaagatgacaatataatgtctaagtagtgacatgtttttgcacatttccagcccctttaaatntccacacacacaggaagcacaaaaggaagcacagagatcctgggagaaatgcccggcccgctcta

SEQ ID NO: 2 - DNA, 237 bp, Homo sapiens
  misc_feature (1)...(237): n = A,T,C or G
ccgcggtggcggccgaggtnccaangtcaagtcaccataatgtntttnacctgtttcacaatgttctacattatcgtggaaatgttgacaaggtctcnagcaaagactggatctncantggntttaaaatacaatnntccatttcggaaattganggaaaaagatctttnncaanctggaagagtcttcttgtaaataggaatcacngggctacacctgctgacgaccttgggtttn

SEQ ID NO: 3 - DNA, 463 bp, Homo sapiens
  misc_feature (1)...(463): n = A,T,C or G
ttagggcgaattggagctccccgcggtggcggccgaggtactatatggacaccaacatggagcanaaaatgagggcagcagtgtccaatctcattccaggtgagaagttgcacagtgtccaataggtgcatacatctccttagtaagtagttgtgattaacaatgaaatagaaatgaaaaatatatttttttatttatgtgtattatatttttcaagcagctaataagttggtaggacataatatttaattcgttggggacctaattatttataaattgaatggttagatatttcttttggcctaagccaccatgaaaaaatcactgaggcgctaagggaaacatgaactaagaagcccttctgagtctctgttttctcaactgtaagatgaanaaacctgctccaccttcctcttgcgattggtgaacggcagtttgagatactgcgcagaatggaccttat

SEQ ID NO: 4 - DNA, 434 bp, Homo sapiens
  misc_feature (1)...(434): n = A,T,C or G
ggagctccccgcggtggcggccgaggtacgcgggggttgtatgtgggttggcattcttgcatgatgggagtggccacctgctttcatattctgaagtcagagtgttccagacagaagaaatagcaagtgccgagaagctggcatcagaaaaacagaggggagatttgtgtggctgcagccgagggagaccaggaagatctgcatggtgggaaggacctgatgatacagaggtgagaaataagaaaggctgctgactttaccatctgaggccacacatctgctgaaatggagataattaacatcactagaaacagcaagatgacaatataatgtctaantagtgacatgttttttgcacatttccagcccctttaaatatccacacacacaggaagcacaaaaggaagcacagagatccctgggagaaatgcccg

SEQ ID NO: 5 - DNA, 332 bp, Homo sapiens
  misc_feature (1)...(332): n = A,T,C or G
ctatagggcgaattggagctccccgcggtggcggccgcccgggcatggtacgcggggaggtgttgttccgtgaatcagcacatcaattgcagcattgtggctaccagggggtcaggatgcggncggtggagccctctggcctttgtgtggtagccgaggactctgtgtcagctgaccgttttccggnaaactttcgtgcgagactcacatcttggaaattcaaatactcaatanctctcgaattctaggaatcttgagaagaggcctggattaangattcanacatgggccctnanatngntatggcattgctggttctaccaacgtgacag

SEQ ID NO: 6 - DNA, 348 bp, Homo sapiens
ccgggcaggtaccagaacttcagcaaaggaagcagacacaggtcggaggaagcctacatagaccccattgccatggagtattacaactgggggcggttctcgaagcccccagaagatgatgatgccaattcctacgagaatgtgctcatttgcaagcagaaaaccacagagacaggtgcccagcaggagggcataggtggcctctgcagaggggacctcagcctgtcactggccctgaagactggccccacttctggtctctgtccccctgcctccccggaagaagatgaggaatctgaggattatcagaactcagcatccatccatcagtggcacgagtccaggaag

SEQ ID NO: 7 - DNA, 188 bp, Homo sapiens
  misc_feature (1)...(188): n = A,T,C or G
ttagggcgaattggagctccccgcggtggcggccgaggtactttttttttttttttttttttttggaccttgggtatcttctgcaggtcaagccatcctgaccactctgagaacttacccagggtgactcanctgtcactccaggtcactcataagcccaacgggtagctccacccgtggacagctgg

SEQ ID NO: 8 - DNA, 603 bp, Homo sapiens
  misc_feature (1)...(603): n = A,T,C or G
cgcggtggcggcccgcccgggcaggtactttttttttttttttttttttttttttttaagggaacgtgaattttaatgagggggcanaccgaggaggtggtggctgcccggagatcagggccaggctgtgctanatggcncctggaaggggggtcacccaagtctccctgctatcatttcaggaggccgacccaagtctccctgctgtcatttcaggaggccgaattttttcccgatcccanagaaggtgtcaaaggcctggttagcantcttgtcgatggtttcctgggtggtcttggccanctggtccatggctttctgccccgcctctgtggcctggtccaccacttgctganctgccgctccggcccgctnacacggcttcctgggcggtcccctccacctgttgcttcaggtcctgcaagcccttgcttgccatggcttcggggtatctgtggagtcntcaagancanctggagccacnttggatcctncccaaagtggcccccgcgtaccttggcccgtntaaaactatgtggaatccccccggcctgcangaattccatatcaaagcttatngatacccggcgaccctcgaggggg

SEQ ID NO: 9 - DNA, 429 bp, Homo sapiens
ccgcggtggcggccgaggtacgcggggtcattactacaggaaaaactgttctcttctgtggcacagagaaccctgcttcaaagcagaagtagcagttccggagtccagctggctaaaactcatcccagaggataatggcaacccatgccttagaaatcgctgggctgtttcttggtggtgttggaatggtgggcacagtggctgtcactgtcatgcctcagtggagagtgtcggccttcattgaaaacaacatcgtggtttttgaaaacttctgggaaggactgtggatgaattgcgtgaggcaggctaacatcaggatgcagttgcaaaatctatgattccctgctggctctttctccggacctacaggcagccagaggactgatgttgcgctgcttccgtgatgtccttcttggctttcatgatggc

SEQ ID NO: 10 - DNA, 338 bp, Homo sapiens
  misc_feature (1)...(338): n = A,T,C or G
cgctgatgttggccangcgagagggccacgttctcctgagtgttcagaagggtcttacagacgtccaggggggtcgtggcggccgcggcgagggccccggccagcccgcctgagatgatgtgggactgcgggttgtaggtccggtgggggttgacctgctcctgcaggaactcataggtgatgaagtggatggactggaaggggatgttcatggtcagctgcgtggtgtagctccggtagaaggcccccaacccctnggtcctccacaccgtccggatgcaactgattgctgaccggtgctgcnagttgtacctgcccgggcggccgctctagaacta

SEQ ID NO: 11 - DNA, 561 bp, Homo sapiens
  misc_feature (1)...(561): n = A,T,C or G
gccgccgggcaggtacaaacttagaagaaaatnggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttaccgtagaccngaaccaatttagaagaatacttgaagctagaaggggaagttggttaaaaatcacatcaaaaagctactaaaaggactggtgtaattttaaaaaaaactaaggcagaaggcttttggaagaagttagaagaatttggaaggccttaaatatagtagcttaatttgaaaaaatgtgaaggacctttcgtaaccggaaggtnatttcaagatnaaagaagtaatttaccaaccttaatggtttttgcctttggggcttttgagtttaaagaattant

SEQ ID NO: 12 - DNA, 367 bp, Homo sapiens
  misc_feature (1)...(367): n = A,T,C or G
gaatnggagctccccgcggtggcggccgangnactcngganagagtanaaacgtttccatgttatttctgcagttgtngacttaggcttatttgtaaagaagcatgctccattgactgccatctctagtcttgcagtgggtggtattaacccatagaaagcaagcagttgtgtatcacatacacaatggntatgatgntaaccanatcanctgtttngttgttcattcgtnatatgttttgtgatanggatgttgggagcacagctctattctgcctgctcagacttaagttagacccttatcttttatattatgtcatgaaaaaagtctcctaaaaattgtgaaactagcctcttgatgagtgatg

SEQ ID NO: 13 - DNA, 464 bp, Homo sapiens
ccgcggtggcggcccgaggtacatggccacaagaggagccctggccaacagtttcaggctggccatcttgggctggggctccctgggcttgcagtggggagggtcaggattcaaattctgggtgacaatttcagtagcaccaagaccagaagacagtgaggccacgctggggagtgggggagtgcttgtatgtgggatgcatggagggaggctgcagtatgtggaggagaaagagaggctgttgtgggctggttcttaagacactcaaggggactcaaagggtcggctccagccatggacactatctaaggctgctcagaggagaatgctggaggagaggaagaggaagtgaaatggtgtgagaacattcttacccctataaataaactatttacacactttaagaaaaggagagccggctctcagtcccgcgtacctgcccgggccggcccgctctagaacta

SEQ ID NO: 14 - DNA, 519 bp, Homo sapiens
  misc_feature (1)...(519): n = A,T,C or G
ccgcggtggcggccgaggtacgcggggacctcagcccagcaattcgtttggagcatgtgaacaccttgagccttgatgagttccagtatgtggtatattatgcagagcattcagagcaaatactctctctccgagcgcttaatccgaacaattgctgccatccgttccttcccacatgataatgtagaggacctcatcagagggggagcagatgtgaactgcactcatggcacactgaagcccttgcactgtgcctgtatggtgtcagatgctgactgtgtggagttacttctggaaaaaggagccgaggtgaatgccctggatgggtataacccgaacagccctccactatgcancagagaaagatgaggcttgtgtggaggtcctattggagtatggtgcaaaccccaatgctttggatggcaacagagataccccacttcactgggtagcctttaagaacaatgctgagtgtgtgcgggctctcctagagagcggggcctctgtcaatgccctgga

SEQ ID NO: 15 - DNA, 422 bp, Homo sapiens
  misc_feature (1)...(422): n = A,T,C or G
aaatccggcgaatngtttctncaccgcggtggcggncgcccgggcaggtacncgggtatgctgcctggacacacgcagatttnctgtctantcacacacacacgcagacatgctgtccggacacacacacgcatgcacagatatgctgtccggacacacacacgcacgcanatatgcttgcctggacacacacacagataatgctgcctcaacactcacacacgtgcagatattgcctggacacacacatgtgcacagatatgctgtctggacatgcacacaccgtgcagatatgctgtccggatacacacgcacgcacacatgcagatatgctgcctgggcacacactttcggacacacatgcacacacaggtgcagatattgctgcctggacacacgcagactgnacgtgcttttgggag

SEQ ID NO: 16 - DNA, 360 bp, Homo sapiens
  misc_feature (1)...(360): n = A,T,C or G
aggagtgcagaaccctgtancccgctgcatcatgtgctgtttcaagngctgcctctggtgtctggaaaaatttatcaagttcctaaaccncaatgcatacatcatgatcgccatctncgggaagaatttntgtgtctcagccaaaaacgcgttcatgctnctcatgcgaaacattgtcagggnggtcgtcctggacaaagtcacagacctgctgntgttctttgggaagctgctggtggtcggaggcgtgggggtcctgtccttcttttttttctccggtcgcatnccggggctgggataaagactttaanagcccccacctnaactattactggctgccccatcatgaccctncantcc

SEQ ID NO: 17 - DNA, 428 bp, Homo sapiens
  misc_feature (1)...(428): n = A,T,C or G
cgcccgggcaggtacattctcatcattaggattggttgttgctgtccttgcacaactggttaaggaaaaatgaattattctgtaatttctgaagaatccaaatcctgtctcttataaagtcagaacagaagggggcaaaaggtggtggtgagcatcaagagagaaaaaaggagaaaattatttacagaaaataggagacangagggagtgtccgcaagaaaagacttcattgtcacttcttcttgccggccctcttggcactggactttgctttgggtttcactggtttggcctttttgggcttggatgcctttgaccggcttggctttgacagtcttgggtttttttggcnttcttgggcgtggcagccaagcttcttcttggccttcttgacccggggtggctttnggttttctttggnttggggg

SEQ ID NO: 18 - DNA, 491 bp, Homo sapiens
  misc_feature (1)...(491): n = A,T,C or G
ggtggcggccgaggtacctcaaaagtggggaaatccccatataactgangacgaaggcagttcagaagttcattgatttgccctaaggttcctcaatttgcaaacgtcaggccaatgatccaaccccaggtatgtttggcagtgaaggaccagttgagtcatagctgcaagtaaccaccctgcagtggtccctatcttggccgttagcttacattgacatttaacactcaaatttactcagtaacaccagctatcatgttttccactaaaactccacagcattctggcaacttttctattttagagcaataaagtaaattgttagcatccctttgacatataaatatttctacaaatagtaattctctagccattcatttggagtatttaaaactcaacattcatagcacattttatggtgacaaagaacttatgttcagaacacaaaaaataagtcgtacctgcccgggcgggcggtcttngaactagtg

SEQ ID NO: 19 - DNA, 114 bp, Homo sapiens
acaaaaaccaatctacctgatgaaaactccgttcccttctcgccagaaacataaaatgcgatggagctacggccaccgctgccgagacaaaatggcgccgacccccgcgtacct

SEQ ID NO: 20 - DNA, 405 bp, Homo sapiens
  misc_feature (1)...(405): n = A,T,C or G
gagctcnccgcggtggcggccgcccgggcaggtactttttttttttctccggtcgcatcccggggctgggtaaagactttaagagcccccacctcaactattactggctgcccatcatgacctccatcctgggggcctatgtcatcgccagcggcttttttcagcggttttcggcatgtgtgtggacacgcccttcctctgcttcctggaagacctggagcggaacaacggctccctggaccggccctactacatgtccaagagccttctaaagattctgggcaagaagaacgaggcgcccccggacaacaagaagaggaaggaagtgacagctccggccctgatccaggactgcaccccacccccaccgtccagccattcaaccttacttcgccttacagggtc

SEQ ID NO: 21 - DNA, 530 bp, Homo sapiens
ctcactatagggcgaattgggagctccccgcggtggcggcccgcccgggcaggtactatatggacaccaacatggagcagaaaatgagggcagcagtgtccaatctcattccaggtgagaagttgcacagtgtccaataggtgcatacatctccttagtaagtagttgtgattaacaatgaaatagaaatgaaaaatatatttttttatttatgtgtattatatttttcaagcagctaataagttggtaggacataatatttaattcgttggggacctaattatttataaattgaatggttagatatttcttttggcctaagccaccatgaaaaaatcactgaggcgctaagggaaacatgaactaagaagcccttctgagtctctgttttctcaactgtaagatgaagaaacctactccaccttcctcttgcgattgttgaacggcagttgagatactgcgcagaatgggaccttattgatggcctacccaacatccattctctactccctctactctgatggcacccg

SEQ ID NO: 22 - DNA, 195 bp, Homo sapiens
  misc_feature (1)...(195): n = A,T,C or G
ccgcggtggcggccgaggtacgcggggttttcagggtcgtaggacgccgttgggcaccacgctcggagaaggacaggacaatggcggccttagggtccccgtcgcacacttttcgaggacttctgcgggagttgcgctacctgagcgcggccaccggccgctctagaactaagtggatccccccggcttgcanga

SEQ ID NO: 23 - DNA, 198 bp, Homo sapiens
  misc_feature (1)...(198): n = A,T,C or G
ctccccgcggtggcggccgaggtacgcggggctctatataagtgggcagnggccgngactgcgcgcagacactgaccttcagcgcctnggctccagcgccatggcgccctccaggaagttcttcgttgggggaaactggaagatgaacggacggaagcagantctgggggagctcatcggcactctnaacgcggccaa

SEQ ID NO: 24 - DNA, 620 bp, Homo sapiens
  misc_feature (1)...(620): n = A,T,C or G
ccaccgcggtggcggccgaggtacagtggcacctgggaaaaggcacctggaaggtttccatgtggcccagcccagcatggaagcagggtgggaactctgctgtgtcgccagcgctcactctactcgagtggctttttgaaagccctaccatgtctgtgtcaggcctgtgctgcttcacatcctacagctgcctaggaaaggccggccacgctccctgtccacacactccctgtccacacactccctgtccacacactccctgtccacaactgcagccgggccctctgcctatgggcacccaatccaagcagctgctccacctttgtttggcatggtgatttgtgttttttctcttggcgcttatgtgtgtgggcttgggacgaagtgctggtatgcacttaggaccttcttgatagctccctgcactttggaacacggagcaaatgaaaaaagggtcggggcttgccctccaccttggacttggaagaagcccacattggagangtgaaggaccccatggtggctctagtgggaaaatacgttagcctcaanctnagganggatgaggccaaccccaanagggagaccttaattgataggggatcangctaaaaaaatgg

SEQ ID NO: 25 - DNA, 349 bp, Homo sapiens
aggtactcgcgctgttcttctgttatcatccagggttcgtcggggtaactgctgttatcctgtagttccctctccactgaaaaagaccggataagggaagcctgatgccgaaggggcttggttcctgggccccctgatgaggatccctcgggctggacaaggggctgcctagcttcataaggtgctggatgcatctgttctgcgttgctccgcagtttgctgtagccatggctcaagggcaccctctggtaatgaggcggggaagaaggaggggaggcgaggggtgacatcgtgggagactgtgtttcctgctgtttatcctcatcgtccattcttttgaaggaatttt

SEQ ID NO: 26 - DNA, 434 bp, Homo sapiens
  misc_feature (1)...(434): n = A,T,C or G
ccgcggtggcggccgcccgggcaggtacgcggggattgtggcagctggaggtctctgcatcgcttatccgtttattagccggaccaagattgcacagctaaagtctggcagagactccacggtatgactgtcctcactgggcctgtccacagtgcgagcgactcctgaggggaacagcgccggagttcaggagtccaagcacaaagcggtcttttacattccaacctgttgcctgccagccctttctggattactgatagaaaatcatgcaaaacctcccaacctttctaaggacaagactactgtggattcaagtgctttaatgactatttatgcgttgactgtgagaatagggagcagngccatgggacatttctaggtgtagagaaagaagaaactgcaatggaaaaatttgtatgatttccatttatttc

SEQ ID NO: 27 - DNA, 480 bp, Homo sapiens
ccgcggtggcggccgaggtacagtggttaatttgtcacaactgactgaggcatcacaacaacagcagcagtcaccactacaagaacaagcacagactttacagcagcagatttcatcaaatatttttccatcaccaaatagtaacaacatgcctggaattcaaggagccacatcttcgccttcaaccacaggctacttttatttcacaacacagcaggaggcacaatgaaccaactgcagaattctcctggctcatctcagcagacatcaggaatgttcttatttggcattcaaaataactgtagtcagcttttaacctctggaccagctacattgcctgatcagttgatggccataagtcagccaggccaaccacaaaacgagggccagccacctgtgacaacacttctttctcagcaaatgccagagaattctccactggcatcctctataaacaccaaccagaacatcgaaaagatt

SEQ ID NO: 28 - DNA, 472 bp, Homo sapiens
  misc_feature (1)...(472): n = A,T,C or G
ctacttagggcgaattggagctccccgcggtggcggccgcccgggcaggtactctggtcatcttcgttcgtttggtcgtgcaaggtgttaactattttcacttcccatatcacaaagttagtccacaggaggagctggtggatcttgtccattatgaggactggttggctttccaggtaggctggatcctnttagattaggagggtctcagtaagaacaggatcaatgcaataaccatccaggctaccaagatnattgtaacactgatgccnttatnaccagagggtcccggtaattnctgaanacactctgtgtctgtgcagtatgattgggactgccgtancanattgntcagtcttctcantgcatngtttcatgagagcaaaacacattnacagggatcaaattccaccttcttgcccattattacccagcttggatttttacttggacctntttttaaaatcccncc

SEQ ID NO: 29 - DNA, 449 bp, Homo sapiens
  misc_feature (1)...(449): n = A,T,C or G
cccgcggtggcggccgaggtactgcgtggccgtccgctgttgtgtggcctgggccgcaaaccgggccacgtggttgacaatgggggagaaattggtaggaccgtagaagcggatgtggggcaggcaagctgagtacgcctgggcaataccatccacacctgagcagaaggggttggtggggttgaagttgatggcaaactcatgggagaccttccagtctgggggtaactgggccccgaatcccagagctggaaacatcttatcactgtcgtagtcctgaatgatctgcccaacagcccagatggccgacagatattcgttggtgcccatagggttgatatantgcaaagaggaagggtcgaggggattcccgttggaggctgtaaagtctattccaacggtgaacatgagctggcagcctcccaggacgtagtcaaggaaggagtagt

SEQ ID NO: 30 - DNA, 400 bp, Homo sapiens
  misc_feature (1)...(400): n = A,T,C or G
ccgcggtggcggccgaggtctgaactgcggggtctctatcgcactgctaggggttctgctgctgggtgcggcgcgcctgccgcgcggggcagaagcttttgagattgctctgccacgagaaagcaacattacagttctcataaagctggggaccccgactctgctggcaaaaccctgttacatcgtcttttctaaaagacatataaccatgttgtccatcaagtctggagaaagaatagtctttacctttagctgccagagtcctgagaatcactttgtcatanagatccagaaaaatattgactgtatgtcaggcccatgtccttttggggaggttcagcttcagccctcgacatcgttgttgcctaccctcaacagaactttcatctgggatgtcaaa

SEQ ID NO: 31 - DNA, 122 bp, Homo sapiens
  misc_feature (1)...(122): n = A,T,C or G
tgatcttcagctgctcctgaactttggcactgtaagggttctctgtatcgtatggtggtgacaaagcttttaaaataacctntcctccagtgcatggaaaaggactggagtatttatnaagt

SEQ ID NO: 32 - DNA, 575 bp, Homo sapiens
acttgtgagcctggactggcttccccggcctcctacatgaaccccttcccggtgctccatctcatcgaggacttgaggctggccttggagatgctggagcttcctcaggagagagcagccctcctgagccagatccctggcccaacagctgcctacataaaggaatggtttgaagagagcttgtcccaggtaagccaccacagtgctgctagtaatgaaacctaccaggaacgcttggcacgtctagaaggggataaggagtccctcatattgcaggtgagtgtcctcacagaccaagtagaagcccagggagaaaagattcgagacctggaagtgtgtctggaaggacaccaggtgaaactcaatgctgctgaagagatgcttcaacaggagctgctaagccgcacatctcttgagacccagaagctcgatctgatgactgaagtgtctgagctgaagctcaagctggttggcatggagaaggagcagagagagcaggaggagaagcagagaaaagcagaggagttactgcaagagctcaggcccctcaaaatcaaagtggaagagtttggaaa

SEQ ID NO: 33 - DNA, 422 bp, Homo sapiens
  misc_feature (1)...(422): n = A,T,C or G
gggagtggagcggtgaacacgtcaggggtggggggcgcaggtcaagctttcaccagtttttaattctttgatggggtaaatttgagcaattttctcgacttgtcgacattcgttattaactgancaggaatcagganaggaacccggtcctctccacacagcccagcanagagcctacgactagatttgcatctttacgtcctgcgcggaggctgctacacacatgcanaagtcatgctggtggcctggacattgaagggagagaagtggatttgggagacatttaggaggcaccgaaagcgaaggaagctcctgctcctcctaaagccgaagccaaagcaaaggctttaaaggccaagaaggcagtgttgaaaggtgtccgcagccacacgcaaaaaagaggatccgcatgtcactcacct

SEQ ID NO: 34 - DNA, 702 bp, Homo sapiens
  misc_feature (1)...(702): n = A,T,C or G
accgcgggggcggccgnggnttcngcctggaggagctcagggtggccggcattcacaagaaggtggcccggaccatcggcatttctgtggatccgaggaggcggaacaagtccacggagtccctgcagaccaacgtgcagcggctgaaggagtaccacaaagggacccaaattcagcggtctgtgcctacaaacttcattaataactgcttgcagattggcagctatctggtcacttgacatatccaatgttgctattttggtctggagaaagttctccctttcttcatctaccttaatttcatgtccatttttaaataattcaaacattttggggatgtcacggccaatggaatttcgagaaagctttggatattttttattcagtttcttccttaatcgattaagttcaggcattatttctggaacagctacgtaaaagtctgcaacaatttcatcatcccaaatcttctgtatcagactaagtgcctcctgcaaatgcagctccattttcttccgctattttgacctctgatgcattctctgtaaatacagcaactttattgatttccgaagccaatgggtatggcaaactaanaacactggtaaatggcttcacgtttttcttnttttccagtgccatatncagtgtcaaaatcaaanaaaacactttgctttggactagttaaagccaanaatttga

SEQ ID NO: 35 - DNA, 597 bp, Homo sapiens
  misc_feature (1)...(597): n = A,T,C or G
acatcttccaggacaaggtaaactctgacatgcactaggtatgtgcagatcccggcccctgccacccagcctcatgcaagtcatccccgacatgaccttcacgaccgcaatgcaaggaggggaagaaagtcacagcactgatgaggacagctgcagaggtggcagtgtgtggacacaggaagtttgggccccctccctgccccagctttcctaggccagaattgtgtttggcagtaattgtctgtttaaaaaaataaaaaggagaggaagcgttcaccgccacaaatcataaaatggacatgactgtggagtcttacagttcagggttctttcattcacgtcccttcctgtctcggtctgcggtctttaccacatcaataggactttttatgcgtccgggttaatttttcactccagtgcgtcctgttgcagggaccggagctgatgggagctgcttctccccatgcctcactggtcccagatcanggcttcagggacagatgatgagtctcaaacgagccancaggggttcttttgggtataaatgggcaattcgncctgtcttaagnctgatgacctcanccgtggtttttggat

SEQ ID NO: 36 - DNA, 457 bp, Homo sapiens
tagggcgaattggagctccccgcggtggcggccgaggtacctgagccaggaggaggcccaggccgtggaccaggagctatttaacgaataccagttcagcgtggaccaacttatggaactggccgggctgagctgtgctacagccatcgccaaggcatatccccccacgtccatgtccaggagcccccctactgtcctggtcatctgtggcccggggaataatggaggagatggtctggtctgtgctcgacacctcaaactctttggctacgagccaaccatctattaccccaaaaggcctaacaagcccctcttcactgcattggtgacccagtgtcagaaaatggacatccctttccttggggaaatgcccgcagagcccatgacgattgatgaactgttgagctggtggtggatgccatctttggcttcagcttcaagggcgatgttcgggaac

SEQ ID NO: 37 - DNA, 433 bp, Homo sapiens
  misc_feature (1)...(433): n = A,T,C or G
agtacttctagaattaattaaggcgggcgcctgcantcccagntacncggnaggctgaggcaggagaatggcgtgaacccangaggcggagcttgcagtgagctgagatcgcgccactgcnctccagcctgggcaacagagtgagactccgtcccgctaanaaatnanaaaanaanaannccnggcccncccnctggngnnttcccanatcccnnttttntgaatttttttnccccccccnnntggncctgttttctactagtgatgatctggtaatatacaatttgtccagtagccagtttgtttttattgtgttttctaaccataagagatcattaaaggcaaagcctgtatgacgctgtacacacacaaaaaaatggtcaccgcaggccatactaccaatgaaatggtaggtaaacaaatcttctggtca

SEQ ID NO: 38 - DNA, 420 bp, Homo sapiens
agtacttctagaattaattaaggcggggacaccacttttcaaaggacttcttggtttcagcataacctaagacagggaattgggagccatcatatgtcacagtgttcagaattcaagcatatttaagggcattttctttgattctcaaagttcagcattcattttgaattgagaagcctatacatttagctgacaaagtgcttatagaatttcttaacaactgaaccattcaaaaggattttttttgtttaaaactggatttcaatgtaagcaaatgaagaaaaaaatatagatttcatttccatagcttcttatccctgtattgaggtaataaattgttttactgacaatttttcctttttctacactaaaacaatatgtgatatatttcccctcttgaagaggcaattcattaaactc

SEQ ID NO: 39 - DNA, 86 bp, Homo sapiens
  misc_feature (1)...(86): n = A,T,C or G
agtacttctagaattaattaaaacagacaattcttaaaaaaaaaaaaaaaaaaaaaaaaaaaaaaaaaaaaaaaaaaaaaaaaaan

SEQ ID NO: 40 - DNA, 428 bp, Homo sapiens
agtacttctagaattaattaaggcggggagttttatctattctttacattggatgcaaaatgtattactcaagaagaggcattcggtatcgaaccatagatgaacatgatgccatcatttaaggaaatccatggaccaaggatggaatacagattgatgctgccctatcaattaattttggtttattaatagtttaaaacaatattctctttttgaaaatagtataaacaggccatgcatataatgtacagtgtattacgtaaatatgtaaagattcttcaaggtaacaagggtttgggttttgaaataaacatctggatcttatagaccgttcatacaatggttttagcaagttcatagtaagacaaacaagtcctatcttttttttttttggctggggtgggggcattggtcacatatgaccagta

SEQ ID NO: 41 - DNA, 246 bp, Homo sapiens
agtacttctagaattaattaaggcgggtccctgcttgtcctatgaactgctcagagctcctgtcagtccagctgggccttctgggttctggcaccatttcgtagccattctctttgtattttaaaaggacgttatgaaagggcttagaccaaaataaatcataatgttacttgagccaccttatatagctgcttggagagtctatgtagttctttctgcatgcattaaaaatgtttagaaatgctt

SEQ ID NO: 42 - DNA, 153 bp, Homo sapiens
  misc_feature (1)...(153): n = A,T,C or G
agtacttctagaattaattaaggcggggtctagctccttatttatctaaataaagttttactggaaaaaaaaaaaaaaaaaaaaaaaaaaaaaaaaaaaaaaaaaaaaaaaaaaaaaaaaaaaaaaaanttaaaaaaaaaaaaaaaaaaaaaa

SEQ ID NO: 43 - DNA, 160 bp, Homo sapiens
agtacttctagaattaattaaggcgggggaaagttagaggaactgaaagtttgggaataggctgaccacatattatgccagtgaccagtatgacaggagatggggccctgctgccagtcatctccactgaataaaaaaaaaaaaaaaaaaaaaaaaaaaa

SEQ ID NO: 44 - DNA, 89 bp, Homo sapiens
agtacttctagaattaattaaggcggtcggttaaaaaaaaaaaaaaaaaaaaaaaaaaaaaaaaaaaaaaaaaaaaaaaaaaaaaaaaa

SEQ ID NO: 45 - DNA, 241 bp, Homo sapiens
agtacttctagaattaattaaggcgggtccctgcttgtcctatgaactgctcagagctcctgtcagtccagctgggccttctgggttctggcaccatttcgtagccattctctttgtattttaaaaggacgttatgaaagggcttagaccaaaataaatcataatgttacttgagccaccttatatagctgcttggagagtctgtgtagttctttctgcatgtattaaaaatgtttagaaa

SEQ ID NO: 46 - DNA, 263 bp, Homo sapiens
  misc_feature (1)...(263): n = A,T,C or G
gccctgggaaacgattgtgaacgcntgaatgaattgatgactaanatccgctgcgggggtcctacagcgcanatggtaatgcccgttctgactggctgggaacggcaccttagcaagatacttaaaaggcgccttctgtgtgccacntgcactccaccctgggcaacagagcaaanaccccatctcaaaaataaataaatatatataaaaaataaaaagctatttctagtttnatttcactataaagttttgcttttattaaa

SEQ ID NO: 47 - DNA, 277 bp, Homo sapiens
  misc_feature (1)...(277): n = A,T,C or G
agtacttctagaattaattaaggcgggcggatcatgaggtcaggagttcgagaccagcctggccaacatggtgaaactccgtgtctactaaaaatacaaaaattggctgggccgtggtggttcgcacctgtagtcccagctactgggggggctgaggcaggagaatggcttgaacccgggagggtagaggttgcagtgagccaagattgtgccactgcactccagcctgggcgactgagcaagactgtctaaaaaaaaaaaaaaataaataaagnaa

SEQ ID NO: 48 - DNA, 393 bp, Homo sapiens
  misc_feature (1)...(393): n = A,T,C or G
gccgtcgttttacaccccgnaanaancnatancnatgatngntgccngtcccnctcnnnanagnaataataaaantaattaangcgggtgattaaatttcagtccattatgattttcctttctcacataattacttttttctttttagacttataagctagcaattacagatttaactacagctatcagcatggacaaaaatagttatacagcattttataacagagcattatgttacaccaagataagggaacttcaaatggcattaacagattatggaattgtgctgcttcttgatgctacagaaactgtnaaactaaataccttccttaatcgtggactcatctacgtagaactaggccagtatggctttgcactagaggattttaaaac

SEQ ID NO: 49 - DNA, 228 bp, Homo sapiens
  misc_feature (1)...(228): n = A,T,C or G
ctncgangntacttnctagaaattaattaagggcggggntagancaaaaaaaaataaaacaacaacaaaaaaaaacnaaaaatanaaaaaaaaaaaanaannnanncaannnaaaantaaaaattnnntatttattttaaantaaaaantatactnaactaattatttnaattaaagaaaaaataantaaaaaaattnataaanaataaatnttaaataaaaaatatt

SEQ ID NO: 50 - DNA, 158 bp, Homo sapiens
agtacttctagaattaattaaggcggggagaggaggctctcaataaataatcgtgtaaccttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 51 - DNA, 112 bp, Homo sapiens
agtacttctagaattaattaaggcgggcaataaaagttgtcatggtgtgtaaaaaaaaaaaaaaaaaaaaaaaaaaaaaaaaaaaaaaaaaaaaaaaaaaaaaaaaaaaaaa

SEQ ID NO: 52 - DNA, 345 bp, Homo sapiens
  misc_feature (1)...(345): n = A,T,C or G
tgggtgccgggccccccctcgagagtacttctagaattaattaaggcgggagaaaaggaaaagacattccagacaaaaagactaacttgtcagaaagccctgtggcggaagggagcttttccaatatgaagaactgagcctggagagatgggatgagggggagtgtcgaaccttttaggctttgtaaaggagttttggttttctcctaatagcaatgggatatcttccaaggaatctcaatcaaaagggagagatggctccgattggaatgtcatccctggctgaagagtagaggaagcaaaaaaaaaaaaaaaaaaaaaanngnnnnnncccnntntttttttt

SEQ ID NO: 53 - DNA, 549 bp, Homo sapiens
cccttagcgtggtcgcggccgaggtacttggtggaccaccatcacaccctcctgtgcaatgggtattggcttgcctggctgattcatgtgggagagtccttgtatgccatagtattgtgcaagcataaaggcatcacaagtggtcgggctcagctactctggttcctacagactttcttctttgggatagcgtctctcaccatcttgattgcttacaaacggaagcgccaaaaacaaacttgaagttgtctgaaagcttgctctacacttttacattcatcctcacccttttttttgtggggtagaggaggtgcagtaatttactcagtgatctttctactttctagaaactgtccttcaaagctctttaagaccccctcgttagtcagttttttctcttatatgctctggttgagcttgaatagaccagttgttacttaagaaagaaacagagaaagattttagcttttcaatcctatttggcagaggacttcagctaccttcttacagtctttggctgtgttggtacctgcccgggcggccgctcga

SEQ ID NO: 54 - DNA, 528 bp, Homo sapiens
  misc_feature (1)...(528): n = A,T,C or G
gatatctgcagaattcgcccttagcgtggtcgcggccgaggtacataaagcagattcaagggttaaaataaaaacagaattttggagtgtggtcaaataaggtgcacagattccagaaccctcagagggcctgctggccctctccagacattctgtgtccgtggtgcaggagctgggcccgtccctaacagctccgcactggcttantgcagtggtgctcacagtttcaggaactactaggtgaagtgtctggctcaagtctgccaagtgtcttcactccatcgtcagaagtggagcactatccctaggttcgattcccatgaaatattttatgatttccatcctctntgcccgctcttccaaataaggccctgtgatgccaacgaagggggcatggttgagggtctaaggctctcattaanggcctaattctgtgtgggatatcaacacatgacagacacttgactgcaacattcaagacatttaaggcagtgggttcatttaatgactacttttccaaaataaata

SEQ ID NO: 55 - DNA, 731 bp, Homo sapiens
  misc_feature (1)...(731): n = A,T,C or G
cccttagcgtggtcgcggccgaggtacctcattagtaattgttttgttgtttcatttttttctaatgtctcccctctaccagctcacctgagataacagaatgaaaatggaaggacggccagatttctcctttgctctctgctcattctctctgaagtctaggttacccattttggggacccattataggcaataaacacagttcccaaagcatttggacagtttcttgttgtgttttagaatggntttcctttttcttagccttttcctgcaaaaggctcactcagtcccttgcttgctcaatggactgggctcccagggcctangctgccttcttttccatgtcccacccatgagccctccactggacagcttantaagcctggcccttcattctggcgctgtgttctttctctgggaaaatcaatacctcttaccttctnttgcatgcaaagatcttaaaggattgtcaaactttcaaaacgttacaagnagaaccnnccannaaggtcctataaaatgccagtaagtgacccttntcaagctgtcaaggcttttaaattaggantttggggatttaaatgctttgtntttttttaaagggaaanaaataagagttgctnnnttttaaaaaatgcaatgtttttttanccaattaaaaatttnnccccaaactttttttaaaaagnaaanaaaaanaccncttttgggagancggnaaaaaaaaaaaa

SEQ ID NO: 56 - DNA, 514 bp, Homo sapiens
  misc_feature (1)...(514): n = A,T,C or G
cccttagcgtggtcgcggccgaggtacaagatgtgcatgcagtccaaggccatgaacgaggcatcccacggccagctgggcatgctggtgttcaggcacgagatagaggctcaccttcgcaaacagaagcagaagacaagtagcaaaaaaacatgaactcccagagaaggattgtgggagacactttttctttccttttgcaattactgaaagtggctgcaacagagaaaagacttccataaaggacgacaaaagaattggactgatgggtcagagatgagaaagcctccgatttctctctgttgggctttttacaacagaaatcaaaatctccgctttgcctgcaaaagtaacccagttgcaccctgtgaagtgtctgacaaaggcagaatgcttgtgagattataagcctaatggtgtggaggttttgatggtgtttacaatacactnagacctgatggttttgtggngctcattgaaaatattcatgaatttaaagagcagtttttggtna

SEQ ID NO: 57 - DNA, 409 bp, Homo sapiens
  misc_feature (1)...(409): n = A,T,C or G
cccttagcgtggtcgcggccgaggtactctttctctcccctcctctgaatttaattctttcaacttgcaatttgcaaggattacacatttcactgtgatgtatattgtgttgcaaaaaaaaaaaagtgtctttgtttaaaattacttggtttgtgaatccatcttgctttttccccattggaactagtcattaacccatctctgaactggtagaaaaacatctgaagagctagtctatcagcatctgacagatgaaattggatggttctcagaaccatttcacccagacagcctgtttctatcctgtttaataaattaatttttggggttcctctaccatgccatagcaaaaccccctgcttccaaattcttgtcaacaattaaaaagtctgntggaccttggaagttt

SEQ ID NO: 58 - DNA, 553 bp, Homo sapiens
  misc_feature (1)...(553): n = A,T,C or G
ttttccnttttttttttttttttttttttttccccnccaagctttatctagncttagactttttaaaaaagtttgggggcagattctgaattggctaaaagacatgcatttttaaaactagcaactcttatttctttcctttaaaaatacatagcattaaatcccaaatcctatttaaagacctgacagcttgagaaggtcactactgcatttataggaccttctggtggttctgctgttacgtttgaagtctgacaatccttgagaatctttgcatgcagaggaggnaagaggtattggattttcacagaggaagaacacagcgcagaatgaagggccaggcttactgagctgnccagtggagggctcatgggngggacatggaaaagaaggcagcctaggccctggggagcccagtccactgagcaagcaagggactgagtgagccttttgcaggaaagggctaanaaaaaggnaaaccattctaaaacacancaagaaactggccaaatgctttgggaactggggttattggccnataatggggccccaa

SEQ ID NO: 59 - DNA, 579 bp, Homo sapiens
  misc_feature (1)...(579): n = A,T,C or G
ccctttcgagcggccgcccgggcaggtactttttttttttttttttntttttttccgcctccccaaagctttatttntnttgantttttaaaaaagtttgggggcanattctgaattggctaaaanacatgcatttttaaaactagcaactnttatttntttcctttaaaaatacatagcattaaatcccaaatcctatttaaagacctgacagnttganaaggtcactactgcatttataggaccttctggnggttctgctgttacgtttgaagtctgacaatccttganaatntttgcatgcanaggaggtaanaggtattggattttcacagaggaagaacacagcgcanaatgaagggccaggcttactgagctgtccagtggagggctcatgggtgggacatggaaaanaaggcagcctaggccctggggagcccagtccactgagcaagcaagggactgantgagccttttgcaggaaanggctaanaaaaaggaaaaccattctaaaacccaacangaaactgtccaaatgctttgggaactgggtttattgcctataaagggtccccaaaa

SEQ ID NO: 60 - DNA, 71 bp, Homo sapiens
ctcgagccggctcgccagtgtgatgggatatctgcagaattcgcccttagccgtggtcgcggcccgatgta

SEQ ID NO: 61 - DNA, 552 bp, Homo sapiens
cccttagcgtggtcgcggccgaggtacctagaaaacagaaacttgagtagacatggtaatgaccagaaaaggctatctttatacatttcttttgctacgcttcaaattcatgtcacctaaaagttgtgaagtgcacaaaacaaatctacttaactgaaaattattttcaatgaatgggatgtttagaactctgtgagggtttttaaggtcttttcgaatagcaaattctaatgaggcttttttaagttggcaatttaaactcatacaagaaataaaaactcaccagtgtggctgggcagaatatatatattttctcaaatattgtttgtttgttttttccctgcactgtatccatggtcccatgatgaaactgttatattgctgatatatttattggaatatgtgggccaacttcctttccactcaacatatggattggtagtttaaaataattcctttctattaagcaaatgtgtggctaaggcacatttaaatagcccattaaaccaatggagatgacaatgtgttaccctcagagaaagcttaattttt

SEQ ID NO: 62 - DNA, 463 bp, Homo sapiens
ccctttcgagcggccgcccgggcaggtactatatggacaccagcatggagcagaaaatgagggcagcagtgtccaatctcattccaggtgagaagttgcacagtgtccaataggtgcatacatctccttagtaagtagttgtgattaacaatgaaatagaaatgaaaaatatatttttttatttatgtgtattatatttttcaggcagctaataagttggtaggacataatatttaattcgttggggacctaattatttataaattgaatggttagatatttcttttggcctaagccaccatgaaaaaatcactgaggcgctaagggaaacatgaactaagaagcccttctgagtctctgttttctcaactgtaagatgaagaaacctgctccaccttcctcttgcgattgtcgaacggcagttgagatactgcgccagaatggacccttattgatggcctac

SEQ ID NO: 63 - DNA, 663 bp, Homo sapiens
  misc_feature (1)...(663): n = A,T,C or G
ccctttcgagcggccgcccgggcaggtacctcattagtaattgttttgttgtttcatttttttctaatgtctcccctctaccagctcacctgagataacagaatgaaaatggaaggacagccagatttctcctttgctctctgctcattctctctgaagtctaggttacccattttggggacccattataggcaataaacacagttcccaaagcatttggacagtttcttgttgtgttttagaatggttttcctttttcttagccttttcctgcaaaaggctcactcagtcccttgcttgctcaagtggactgggctccccagggcctaggctgccttcttttccatgtccacccatgagccctccactggacaagctcagtaagcctggcccttcattctgcgctgngttcttctctgtgaaaatccaatacctcttaccttctctgcatgcaaagattctcaaggattgcagacttcaaacgtaacagcagaaccaccagaaggtcctataaatgcagtagtgaccttttcaagctgtcaggcctttaaatangaatttgggaatttaatgctatgtatttttnaaaggaaagaaataagaagttgcttagntttnaaaatgcatgtcttttancccaattaaaaatttgcccccaaactt

SEQ ID NO: 64 - DNA, 269 bp, Homo sapiens
  misc_feature (1)...(269): n = A,T,C or G
ngatatctgcanaattcgccctttcgagcggccgcccgggcaggtacaactttttaaatagggaatatgatagcttngcatggtggtgtgcacctatagcccccactgcctggaaagctggggtgggagaatcgcttgagtccaggagtttgaggttacagtgatccacgatcnngccactacactccagcctgggcancagagcaagaccctgtntcaaagcataaaatggaatnacatatcaaatgaaacanggaaaatgaagctga

SEQ ID NO: 65 - DNA, 194 bp, Homo sapiens
  misc_feature (1)...(194): n = A,T,C or G
ccctttcgagcggccgcccgggcaggtacctttttttttttttttttttttccgtctccccaaagctttatctgtcttgactttttaaaaaagttngggggcanattntgaattggntaaaagacatgcatttttaaaactagcaactcttatttctttcctttaaaaatacatagcattaaatcccaaatcct

SEQ ID NO: 66 - DNA, 69 bp, Homo sapiens
gcatgctcgagccggccgccagtgtgatgggatatctgcagaattcgccctttcgagcggcccgcccgg

SEQ ID NO: 67 - DNA, 509 bp, Homo sapiens
ccctttcgagcggccgcccgggcaggtacaagctatcttttgctccaaaacagttctgaaggttttatttatattttatcttatcccgagggaccaacagcaggcatacctttgccaggccttcttgcagaaagacacagagccgtaaaggcaaaaataaaattgcaataaagtatatggtattgggggcagggagaaccagaaaccctcaaagagaaccaatttgtagcacgttcttttttaaggctctacccctgtagaagtaagaaactagcctgcctttttagccatatgagagtttcctccagagccatcttccaaagtagcagacttggccaagttgcccaatgccaaataagtgagttggaagttcgtttgcttcaaacacactgcacttagaaaccagacttgaaataatcgaagccccacagaaaagcttcatgaaacgaagtgttactttcctagagaataagaaagtcacaagattgaggagtctgttctaaagttct

SEQ ID NO: 68 - DNA, 716 bp, Homo sapiens
  misc_feature (1)...(716): n = A,T,C or G
ccctttcgagcggccgcccgggcaggtacctcattagtaattgttttgttgtttcatttttttctaatgtctcccctctaccagctcacctgagataacagaatgaaaatggaaggacagccagatttctcctttgctctctgatcattctctctgaagtctaggttacccattttggggacccattataggcaataaacacagttcccaaagcatttggacagtttcttgttgtgttttagaatggttttcctttttcttagccttttcctgcaaaaggctcactcagtcccttgcttgctcagtggactgggctccccagggcctaggctgccttcttttccatgtccacccatgagccctccactggacaagctcagtaagcctggcccttcattctgcgctgtgttcttcctctgtgaaaatccaatacctcttacctcctctgcatgcaaagattctcaaggactggcagacttcaaacgtaacagcagaaccaccagaaaggcctataaatgcagtagtgaccttctcaagctgcangtctttaaataggatttgggaattaatgctatgtanttttaaanggaaagaaataagaagttgctagttttaaaaatgcatgtttttaagccaattcaaaaattggccccaactttttttaaaaagtcaagacaagataaagcntttggggagaacggaaaaaaaaaaaaaaa

SEQ ID NO: 69 - DNA, 477 bp, Homo sapiens
  misc_feature (1)...(477): n = A,T,C or G
cccttagcgtggtcgcggccgaggtactcatgtattttttttttccagatctctttccccaagttgctattgtaagagtattctgctgcgtgtggatgcagttatacacattaaagcagatctggagtctgaagtagctataaagcagctataaaacagaaatacatgcatagctgcagaaaccatgataggtagaggacttttcttttggttttgttttgttttgttttgttttgtttttggttttacagagaagagatttttattcaaagaaaaaaattccagtgaattgtgcacaaatgctggtttttacaccatcctaaagaaaaactttacaaggggtgttttggagtanaaaaaaggttataaagttggaatcttaaattgtnaaattaaccattgagtgtcaaggntctaaaagcagaacttattttgtgcaatgaacataangaaagactactgtatagggtttttttt

SEQ ID NO: 70 - DNA, 380 bp, Homo sapiens
  misc_feature (1)...(380): n = A,T,C or G
ccctttcgagcggccgcccgggcaggtactcagacaaatacaacgtgcccatttcgtcagatattgcgcagaaccaagaattttataagaacgcagaagttagaccaccatttaccgtatgcatntttaattaaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtggcgagtagaaaacgttaaagggggcagtatgggacagtggattgaagtangaattccaaaaacgaagggcccaaaaaggatcagtgggtaacccttt

SEQ ID NO: 71 - DNA, 377 bp, Homo sapiens
  misc_feature (1)...(377): n = A,T,C or G
ntnttagcgtggtcgcggccgaggtactttttgctttancagatagatagggcatccaatacaactgaaacaacctgataacaaattaattttatttttcaataaaaaggaatgctctggttttttaactggctccttgaggaagccagaagatggcatctgctctttaaacaggcttctcttatctgatttgagacacaaatccaccaagnattctttcttacagtggaagtaggcaccacccctcagatcacgaggcccctaggnaagaagcctgtgggaaatcagccctgtgatgttgtgggtgggtgggccagcacctagaagacacatggtnggttgtgcaacataaaatccccttcaagcggactcagtgg

SEQ ID NO: 72 - DNA, 379 bp, Homo sapiens
  misc_feature (1)...(379): n = A,T,C or G
cccttagcgtggtcgcggccgaggtactctggtcatcttcgttcgtttggtcgtgcaaggtgttaactattttcacttcccatatcacaaagttagtccacaggaggagctggtggatcttgtccattatgaggactggttggctttccaggtaggctggatcctcttagattaggagggtctcagtaaagaacaagatcaatgcaantaaccatccaggctaccaagatcattgttaacactgatgccattatcaccagagggtcccggtaaattccttgaagacactctgtgtctgttgcangtangactgggactnccgtaacagattgatcaagntcttctcanttgcatgttcatggagagcaaacacattcac

SEQ ID NO: 73 - DNA, 403 bp, Homo sapiens
  misc_feature (1)...(403): n = A,T,C or G
cccttagcgtggtcgcggccgaggtcggccgaggtactttattttcaaaacactcatatgttgcaaaaaacacatagaaaaataaagtttggtggaggtgctgactaaacttcaagtcacagacttttatgtgacagattggagcagggtttgttatgcatgtagagaacccaaactaatttattaaacaggattagaaacaggctgtctgggtgaaatggttctgagaaccatccaattcacctgtcagatgctgatagactagctctttcagatgtttttttctaccagttcagaagatngggtttaaatngacctagttccaatgggggaaaaaagcaagaatgggatttcacaaaacccaaggtnatttttaaaccaaaagacccctttttttttggca

SEQ ID NO: 74 - DNA, 378 bp, Homo sapiens
cccttagcgtggtcgcggccgaggtaccatgctgacttcttggtatcttttaaggcctaattttcccttccttgagattactgtagtgtgttccagctaatttctatttggaaacgagttggaacagctgaaaactgggtattattgaaggcaaagcagcctcacgtcagttttttatcagctcatttgggaagtttttttttttttttttttaattaattagaaagtaggctgggcacggtggctcatgcctataatcccagcacttggggaggccgaggatctcctctctggtggatcacttgagggcaggagttaagagaccatcctggccaacatgatgaaaccctgtctctactaaaaatacaaaaagtagct

SEQ ID NO: 75 - DNA, 385 bp, Homo sapiens
  misc_feature (1)...(385): n = A,T,C or G
cccttagcgtggtcgcggccgaggtacagaatttattatgaaatagcttaatggcaagtggtaatttagaagaattaagttatcagataggagatatattaaaatatttaaaaattggatatattcttgaagcccttttacacaagtaatttctataatttgattgtaatgaaagtataatataccttgttactattatcaggattaatttttgaaagtagaattccttaatcaagccaaggttaatgctgctttataggaaattaatcaggtagtttaacactagagctcattagcccaacctgtatgtagcacaaaaataaatcattctctgataaatnccntattaaaatantattttttaattcatacctttttaaaataa

SEQ ID NO: 76 - DNA, 691 bp, Homo sapiens
  misc_feature (1)...(691): n = A,T,C or G
cccttagcgtggtcgcggccgaggtactttttttttttttttttttttttccgtctccccaaagctttatctgtcttgactttttaaaaaagtttgggggcagattctgaattggctaaaagacatgcatttttaaaactagcaactcttatttctttcctttaaaaatacatagcattaaatcccaaatcctatttaaagacctgacagcttgagaaggtcactactgcatttataggaccttctggtggttctgctgttacgtttgaagtctgacaatccttganaatctttgcatgcagaggaggtaagaggtattggattttcacagaggaagaacacancgcaaaatgaagggccaggcttactgagctgtccagtggagggctcatgggtgggacatggaaaagaaggcagcctaggccctggggagcccantccactgagcaagcaagggactgagtgagcctttncaggaaaaggctaanaaaaaggaaaaccattttaaaacacaacaagaaacttgtccaaatgctttgggaaccgngtttattgcctataatgggtcccccaaaatgggtaacctaaactttaaaaganaatgaacccnanagcnnaaggaaaaatctggcttgcccttccattttcattctnttatnttaaggngacctttttnangggganccttttn

SEQ ID NO: 77 - DNA, 697 bp, Homo sapiens
  misc_feature (1)...(697): n = A,T,C or G
cccttagcgtggtcgcggccgaggtactttttttttttttttttttttccgtctccccaaagctttatctgtcttgactttttaaaaaagtttgggggcagattctgaattggctaaaagacatgcatttttaaaactagcaactcttatttctttcctttaaaaatacatagcattaaatcccaaatcctatttaaagacctgacagcttgagaaggtcactactgcatttataggaccttctggtggttctgctgttacgtttgaagtctgacaatccttgagaatctttgcatgcagaggaggtaagaggtattggattttcacagaggaagaacacagcgcaggatgaagggccaggcttactgagctgtccagtggagggctcatgggtgggacatggaaaagaaaggcagcctaagccctggggagcccaatccgctgagcaagcaagggactgantgagccttttgcaggaaaaggcttanaaaaangaaaaccattnttaaaaacaacaagaaacttttccaaatgctttnggaaccggggttattggccataaatgggncccccaaatgggtaanccaaactttaaaaaanatganccgaaancaaagggnaaaatctgggtggcccttccatttnattntngtannncaaagggaacctggnnnaaggggggnccttttnaaaaaaa

SEQ ID NO: 78 - DNA, 582 bp, Homo sapiens
  misc_feature (1)...(582): n = A,T,C or G
cccttagcgtggtcgcggcccgaggtacttttttttttttttttttttttttcccgcctccccaaagctttatctgtcttgactttttaaaaaagtttgggggcagattctgaattggctaaaagacatgcatttttaaaactagcagctcttatttctttcctttaaaaatacatagcattaaatcccaaatcctatttaaagacctgacagcttgagaaggtcactactgcatttataggaccttctggtggctctgctgttacgtttgaagtctgacaatccttgagaatctttgcatgcagaggaggtaagaggtattggattttcacagaggaagaacacagcgcanaatgaagggccaggcttactgagctntccagtggagggctcatgggtgggacatggaaaanaaggcagcctangccctggggagcccantccactgagcaagcaagggactgantgagccttttgcaggaaaaggcttaaaaaaaggaaaaccattctaaaacacaacaaggaaactgtccaaatgctttgggaactgggtttatttgcctattatggggtccccaaaaa

SEQ ID NO: 79 - DNA, 580 bp, Homo sapiens
cccttagcgtggtcgcggccgaggtactacaaaaacagaataattttgaagttttagaataaatgtaatatatttactataattctaaatgtttaaatgcttttctaaaaatgcaaaactatgatgtttagttgctttattttacctctatgtgattatttttcttaattgttattttttataatcattatttttctgaaccattcttctggcctcagaagtaggactgaattctactattgctaggtgtgagaaagtggtggtgagaaccttagagcagtggagatttgctacctggtctgtgttttgagaagtgccccttagaaagttaaaagaatgtagaaaagatactcagtcttaatcctatgcaaaaaaaaaatcaagtaattgttttcctatgaggaaaataaccatgagctgtatcatgctacttagcttttatgtaaatatttcttatgtctcctctattaagagtatttaaaatcatatttaaatatgaatctattcatgctaacattatttttcaaaacatacatggaaatttaacccaattgtctcatataaggttttatttgaattg

SEQ ID NO: 80 - DNA, 596 bp, Homo sapiens
  misc_feature (1)...(596): n = A,T,C or G
ccctttcggcggcccgcccgggcaggtacctggggtctcagggttgctctgggcctgatcatccactcagatctgtaaggaggatttgcaggatccatttagaaagatcctcccttacttccacaagcatggcctttggctcttaaatacctgtgctggggttttgtaattatagaaacaacaggaaccaaaactcattaatgttgagctacaaaccagagggaagcttctttctcaaaacagggctcaggcctagaaaaatctagttttctgaaatcgctagccagcaacagcactgagatggccatcccagaaacaaggccaacacagaagcacccataaaggccgctggaggttgggacaaagagatccttgctgtccttacagaccccctgacttccaaggagctcccctcttacccagcctggcctgccttctccacagggtagctgatcgtcagcatcatcttcaatggtgttgccaaaagcactcaantgctcctgccatccctgtccatcttcaacatgaaaaggagaggttagcacttcaacctgggcgactgagcaaganttcatcttaaaaaaaaaaaaaaaatt

SEQ ID NO: 81 - DNA, 209 bp, Homo sapiens
  misc_feature (1)...(209): n = A,T,C or G
cccttagcgtggtcgcggccgcccgggcaggtactttttttttttttttttttttttaccggctccccaaagctttatctgtcttgactttttaaaaaagtttgggggcanattctgaattggctaaaagacatgcatttttaaaactagcaactcttatttctttcctttaaaaatacatagcattaaatcccaaatcctatttaaag

SEQ ID NO: 82 - DNA, 46 bp, Homo sapiens
  misc_feature (1)...(46): n = A,T,C or G
atctgcagaattcgcccttancgtggtcgcggccgaggtacttttt

SEQ ID NO: 83 - DNA, 601 bp, Homo sapiens
  misc_feature (1)...(601): n = A,T,C or G
cccttagcgtggtcgcggcccgaggnaccatgatatcatgtatcctgcttggacattntgggaaagggggaccagctggttggccaatttatcctacaggtcttggacggngggacctcttcaaaagaagatctggtaagggcaagcagcacaagtggccatggaaaaagaaaaactctacannatatttccgaggatcaaggacaagtccaaaacgagatcctctcattcttctgtctcggaaaaacccaaaacttgtttgatgcagaatacaccaaaaaccagcctggaaatctatgaaagatccttaggaaangccagctgctaaggatgtccatcttgtggatcactgcaaatacaagtatctgtttaattttcgaggcgtaactgcaagttttccggttaaacacctcttcctgtgtggctcacttggtttccatgttnggtgatgaagtggctagaattcttctatccacagcttgaagccntgggttcactatattccaagtcaaaacagatctcttcaatgtccaanagctgttacaattttgaaaaaccaaatgatgatgtaacttcaagaaaattgcttgaaagggggaag

SEQ ID NO: 84 - DNA, 570 bp, Homo sapiens
  misc_feature (1)...(570): n = A,T,C or G
cccttagcgtggtcgcggccgaggtaccacaaaaacagaataattttgaagttttagaataaatgtaatatatttactataattctaaatgtttaaatgcttttctaaaaatgcaaaactatgatgtttagttgctttattttacctctatgtgattatttttcttaattgttattttttataatcattatttttctgaaccattcttctggcctcagaagtaggactgaattctactattgctaggtgtgagaaagtggtggtgagaaccttagagcagtggagatttgctacctggtctgtgttttgagaagtgccccttagaaagttaaaagaatgtagaaaagatctcagtcttaatcctatgcaaaaaaaaaaatcaagtaattgttttcctatgaggaaaataaccatgagctgtatcatgctacttagcttttatgtaaatatttcttatgnctcctctattaagaagtatttaaaatcatatttaaatatgaatctattcatgctaacattattttcaaaacatacctggaaattaacccagaatggctacatataagggtt

SEQ ID NO: 85 - DNA, 724 bp, Homo sapiens
  misc_feature (1)...(724): n = A,T,C or G
ccctttcgagcggccgcccgggcaggtacctcattagtaattgttttgttgcttcatttttttctaatgtctcccctctaccagctcacctgagataacagaatgaaaatggaaggacagccagatttctcctttgctctctgctcattctctctgaagtctaggttacccattttggggacccattataggcaataaacacagttcccaaagcatttggacagtttcttgttgtgttttagaatggttttcctttttcttagctttttcctgcaaaaggctcactcaagtcccttgcttgctcagtggactgggctccccagggcctaggctgccttcttttccatgtcccacccatgagccctccactggacagctcagtaagcctgacccttcattctgcgctgtgttcttcctctgtgaaaatccaatacctcttacctcctctgcatgcaaagattctcaaggattgtcagacttcaaacgngacagcagaaccaccagaaggtcctataaatgcagtaagtgaccttctcaagctgtcaggtctttaaataggatttgggatttaatgctatgtatttttaaaggaaagaaataagaagttgctagttttaaaaatgcatgtcttttagccaatttagaatctggccccaaacttttttaaaaagtcaaagacagataaagctttngggagacggaaaaaaaaaaaaaaaaaaaaagt

SEQ ID NO: 86 - DNA, 51 bp, Homo sapiens
cgccagtgtgatgggatatctgcagaattcgccctttcgcggccgcccggg

SEQ ID NO: 87 - DNA, 510 bp, Homo sapiens
  misc_feature (1)...(510): n = A,T,C or G
ccctttcgagcggccgcccgggcaggtacagtcatcccactacctggctatttcattacttggtgctctagacaagctccaagaactgactggatcttggcttgctctgtttctgtcattgctaatataatatggaaaacattgctgaaaagaacagagatggccatggatatggctaggttaggtattcatatccaaatatctgaactctaacctaatgtggatatgattctgtagcattatattaaaagctatgatgatgcaatgcaggaaataacctttcattctcccccctagaggatcacgacaggtgctttcaatgcctgccttatctatgggacaagtagtgtgattcttcaagtgagaagtgaaagcctttgggggatttgagtcangaaggggaacatggctaaattgcctggaaactctgccaacaagtctgcgggtagattctacttgtctctgggataaaaaaatctgtgctcaatgaaacttattgtgtttggaaaa

SEQ ID NO: 88 - DNA, 59 bp, Homo sapiens
  misc_feature (1)...(59): n = A,T,C or G
tgctcgagccggcccgccagtgcganggatatcngcacaattccccctttcaagcggcc

SEQ ID NO: 89 - DNA, 255 bp, Homo sapiens
  misc_feature (1)...(255): n = A,T,C or G
ccgcggtggcggccgaggtaccacaagatattatgcatgctccaatcgagcaaaatgtccatcataacaaatataaaatgtttccagaaagtatcatagcgtggtttcccatctcctgtatggcttaggctccaaggtctgtgagttaatgctgttattacatcccgatctgtttgtctcatcttgtagataacttctggcaagaaagaacagaccacactattattatacagttgaggaagctctagaactagn

SEQ ID NO: 90 - DNA, 683 bp, Homo sapiens
  misc_feature (1)...(683): n = A,T,C or G
ccgcggtggcggccgaggtacctcattagtaattgttttgttgtttcatttttttctaatgtctcccctctaccagctcacctgagataacagaatgaaaatggaaggacagccagatttctcctttgctctctgctcattctctctgaagtctaggttacccattttggggacccattataggcaataaacacagttcccaaagcatttggacagtttcttgttgtgttttagaatggttttcctttttcttagccttttcctgcaaaaggctcactcagtcccttgcttgctcaagtggactgggctccccagggcctaggctgccttcttttccatgtcccacccatgagccctccactggacagctcagtaagcctggcccttcattctgcgctgtgttcttcctctgtgaaaatccaatacctcttacntcctctgcatgcaaagattctcaaggattgtcagacttcaaaacgtnacagcagaaccaccagaaaggtcctataaaatgcagntagtggaccttcttcaagctgtcagggtcttttaaataggatttgggggatataatggctattgtatttttaaaaagggaaagnaaatnaagagtttgctagattcttaaaaaangccatggtctttttanccaatttnaaaaatnttgccccccaacttttttt

SEQ ID NO: 91 - DNA, 74 bp, Homo sapiens
tcgagcggcccgcccgggcaggtacttttttttttttttttttgggattttttaggtagtgggtgttgagcttg

SEQ ID NO: 92 - DNA, 520 bp, Homo sapiens
cgaggtacgttcttctttcccagtttctctacccttagcttatcctcaccctcattttgccctgctggctgctcaaactatgcaacagattcggcatcctcgcttacccatggcccagtttggaggaaccttctcaccttctcctaacacatggggaccattcccagtgagacctgtgaatcctggcaacacaaatagctctccaaagcataataacacaagccgtctacctaaccagaacgggactgttttaccctcagagtctgctggactagctactgccagttgtcctatcactgtctcttctgtagttgctgccagtcagcaactgtgtgtcactaatacccggactccttcatcagtcagaaagcagttgtttgcctgtgtgcctaagacaagtcctccagcaacagtgatttcttctgtgacaagcacttgtagttccctgccttctgtctcctctgcacctatcactagcgggcaagctcccaccacatttctacctgcaagtacctgcccg

SEQ ID NO: 93 - DNA, 382 bp, Homo sapiens
  misc_feature (1)...(382): n = A,T,C or G
ccgcggtggcggccgcccgggcaggtaccgatacagaagaagctgcgttttgaagacaccctggagtttgtagggtttgatgcgaagatggctgaggaatcctcctcctcctcctcctcatcttcaccaactgctgcaacatctcagcagcagcaacttaaaaataagagtatattaatctcttctgtggcttcggtgcatcatgcaaacggcctagccaaatcttctaccaccgtctctagctttgctaacagcaaacctggctctgctaagaagttagtgatcaagaactttaaagataagcctaaattaccagaaaactacacagatgaaacctggcaaaaactgaaagaagcagtggaagctattcanaatagtacct

SEQ ID NO: 94 - DNA, 396 bp, Homo sapiens
  misc_feature (1)...(396): n = A,T,C or G
ccgcggtggcggccgaggtctatatggaccacnaacatgggagcagaaaaatgagggcagcagtgtccaatctcattccaggtgagaagttgcacagtgtccaataggtgcatacatctccttagtaagtagttgtgattaacaatgaaatagaaatgaaaaatatatttttttatttatgtgtattatatttttcaagcagctaataagttggtaggacataatatttaattcgttggggacctaattatttataaattgaatggttagatatttcttttggcctaagccaccatgaaaaaatcactgaggcgctaagggaaacattgaactaagaagcccttctgagtctctgttttctcaactgtaagatgaagaaacctgctccaccttcct

SEQ ID NO: 95 - DNA, 379 bp, Homo sapiens
cgcccggggcaggtaccttagtgaggctcaaaaggattcttttgggtctattttacgccttatctttgaaattcaccactcgggtgagaaaggtgacattgtagtctttctggcctgtgaacaagatattgagaaagtctgtgaaactgtctatcaaggatctaacctaaacccagatcttggagaactggtggttgttcctttgtatccaaaagagaaatgttcattgttcaagccactcgatgaaacagaaaaaagatgccaagtttatcaaagaagagtggtgttaactactagctctggagagtttttgatctggagcaactcagtcagatttgttatcgatgtgggtgtggaaaagaaaaaaggtgtcctcggc

SEQ ID NO: 96 - DNA, 779 bp, Homo sapiens
  misc_feature (1)...(779): n = A,T,C or G
cgaggtacctgtatgaggtctcccagcccgaaggactaggccaagccctctgtgtgccatctccaatgagaaggaatcctgccctcacctcacccttttccaacttgcccagggaagtggaggttccctctttcctttcctcttgcaggccatccatgactttagagaacagacacaagtgtatccagctgtccacgggtggagctacccgttgggcttatgagtgacctggagtgacagctgagtcaccctgggtaagttctcagagtggtcaggatggcttgacctgcagaagatacccaaggtccaaaagcacgaaggtctgcggaaagttctggttgtcggctggcaccacggttacacctataatcgagcactttgggaggccaagacaggaggatcgcttgagaccaagagtttgagcctgcggtgaagctgtgaatgcaccacggcactcaagcctggcaatgtagcaagatcctgtctctacaagaaaattttttaaaaatgagccaagtgtgggggtgcatgcctgtagtttccagctacttnaggacacttacntanganggttggtttgaaactgaaaagttggaggcttgcaatnanccttgaatgccccantggccttcaacctggggcgaaaanaaccaagaccccatnttaaaaaaaaaaaaanttntngggtngnnattgaattgggaaaaaaaaaaagcttggagctttttgcctttnggccnaggngancattgnaatttgggattttngaanggaatggg

SEQ ID NO: 97 - DNA, 535 bp, Homo sapiens
  misc_feature (1)...(535): n = A,T,C or G
cccttagcgtggtcgcggccgaggtacccgttgctgctgccatgtgtgtgcttaaaacagggttcctttttgtagcatcagaatttggaaaccattacttatatcaaattgcacatcttggagatgatgatgaagaacctgagttttcatcagccatgcctctggaagaaggagacacattcttttttcagccaagaccacttaaaaaccttgtgctggttgatgagttggacagcctctctcccattctgttttgccagatagctgatctggccaatgaagatactccacagttgtatgtggcctgtggtaggggaccccgatcatctctgagagtcctaagacatggacttgaggtgtcagaaatggctgtttctgagctacctggtaaccccaacgctgtctggacagtgcgtcgacacattgaagatgagtttgatgcctacatcattgtgtctttcgtgaatgccaccctagtgttggccattggagaaactgtagaagaagtgactgactctnggttcctggggaccac

SEQ ID NO: 98 - DNA, 231 bp, Homo sapiens
  misc_feature (1)...(231): n = A,T,C or G
aggtactctcgtttcagctgggctcttatggccaaccgctcggcttgcgcccgccgggtttccggantatatgttgtattcggctgggtcgagggtctcaggcagagtgcgcaggctcgacggcttatactttgggaacgacatcttggcgaaccagggcgcaattgcgcctgcgcgattctgaggccctttgtctatgctgaccttcagcttcccccgcgtacctgcccg

SEQ ID NO: 99 - DNA, 229 bp, Homo sapiens
  misc_feature (1)...(229): n = A,T,C or G
ccgcggtggcggccgcccggncaggtacgcggggctgggcgcggggaactgaaagccggaaggggcaagacgggctcagttcgtcatggggctgtttggaaagacccaggagaagccgcccaaagaactggtcaatgagtggtcattgaaggtaagaaaaggaaatgagagttgttgacaggcaaataagggatatccangaaaaaaaaaaaaaaaaaaanagtacctt

SEQ ID NO: 100 - DNA, 513 bp, Homo sapiens
  misc_feature (1)...(513): n = A,T,C or G
cgaattggagctccacccgcggtggcggcccgcccgggcaggtactttttgctttagcagatagatagggcatccaatacaactgaaacaacctgataacaaattaattttatttttcaataaaaaggaatgctctggttttttaactggctccttgaggaagccagaagatggcatctgctctttaaacaggcttctcttatctgatttgagacacaaatccaccaagattctttcttacagtggaagtaggcaccacccctcagatcacgaggcccctaggaagaagcctgtggaaatcagcctgtgatgtgtggtggtgggccagcacctagaanacacatggtggttgtgcacataaatcccttcagcggactcagtggaaggatgagaattctgaaagttcatgcacattttattgagtaggtaatataaaaatgctttttctttttcatttgttacaagtgcatgctttgantgcccaccatnttcagaagtcaaaattacaaaagg

SEQ ID NO: 101 - DNA, 658 bp, Homo sapiens
  misc_feature (1)...(658): n = A,T,C or G
ccgcggtggcggccgcccgggcaggtacgcgggcggggatgggagccacgcctgaactagagttcaggctggatacatgtgctcacctgctgctcttgtcttcctaagagacagagagtggggcagatggaggagaagaaagtgaggaatgagtagcatagcattctgccaaaagggccccagattcttaatttagcaaactaagaagcccaattaaaaagcattgtggctaaagtctaacgctcctctcttggtcagataacaaaagccctccctgttggatcttttgaaataaaacgtgcaagttatccaggctcgtaacctgcatgctgccaccttgaatcccaaggagtatctgcacctgnaatanctctccacccctctctgcctccttactttctgtgcaanatnacttcctgggttaacttccttctttccatccacccacccactgaaatctctttccaaacatttttccattttcccacaaatngnctttgattaactntcctctctccatgcctncaaanctccaaatttttggggaaagctgtaccttcanccnctctaaaactaatgnatccccccgnctncaagaattcnatatcaaacttatcaataccntcacctcaaaggggggcccantacccaacttttn

(19) United States
(12) Patent Application Publication
(10) Pub. No.: US 2002/0168638 A1
(43) Pub. Date: Nov. 14, 2002

(54) Compositions, kits, and methods for identification, assessment, prevention, and therapy of human prostate cancer

(76) Inventors: Robert Schlegel, Auburndale, MA (US); Wilson Endege, Norwood, MA (US); John Monahan, Walpole, MA (US)

Correspondence Address: LAHIVE & COCKFIELD, 28 STATE STREET, BOSTON, MA, 02109, US

(21) Appl. No.: 09/768,827

(22) Filed: Jan. 24, 2001

Related U.S. Application Data
(60) Provisional application No. 60/178,525, filed on Jan. 24, 2000.
(60) Provisional application No. 60/183,245, filed on Feb. 17, 2000.
(60) Provisional application No. 60/190,139, filed on Mar. 16, 2000.
(60) Provisional application No. 60/208,126, filed on May 31, 2000.
(60) Provisional application No. 60/219,705, filed on Jul. 18, 2000.
(60) Provisional application No. 60/255,160, filed on Dec. 13, 2000.

(51) Int. Cl. C12Q 1/68; G01N 33/574

(52) U.S. Cl. 435/6; 435/7.23

(57) ABSTRACT

The invention relates to compositions, kits, and methods for detecting, characterizing, preventing, and treating human prostate cancers. A variety of markers are provided, wherein changes in the levels of expression of one or more of the markers is correlated with the presence of prostate cancer.

### RELATED APPLICATIONS

The present application claims priority to U.S. provisional patent application serial No. 60/178,525, filed on Jan. 24, 2000, U.S. provisional patent application serial No. 60/183,245, filed on Feb. 17, 2000, U.S. provisional patent application serial No. 60/190,139, filed on Mar. 16, 2000, U.S. provisional patent application serial No. 60/208,126, filed on May 31, 2000, U.S. provisional patent application serial No. 60/219,705, filed on Jul. 18, 2000, and U.S. provisional patent application serial No. 60/255,160, filed on Dec. 13, 2000, all of which are expressly incorporated by reference.

### FIELD OF THE INVENTION

The field of the invention is prostate cancer, including diagnosis, characterization, management, and therapy of prostate cancer.

### BACKGROUND OF THE INVENTION

The increased number of cancer cases reported in the United States, and, indeed, around the world, is a major concern. Currently there are only a handful of treatments available for specific types of cancer, and these provide no absolute guarantee of success. In order to be most effective, these treatments require not only an early detection of the malignancy, but also a reliable assessment of the severity of the malignancy.

Carcinoma of the prostate (PCA) is the most frequently diagnosed cancer in men in the United States, and is the second leading cause of male cancer deaths (Karp et al., 1996, Cancer Res. 56:5547-5556). The acute susceptibility of this organ to cancer in men is not understood. Skenes glands represent a tissue in females that is homologous to the male prostate, but not a site where significant neoplastic transformation is observed.

An unusual challenge presented by prostate cancer is that most prostate tumors do not represent life threatening conditions. Projections from autopsy surveys indicate that as many as 11 million American men have prostate cancer (Dhom, 1983, J. Cancer Res. Clin. Oncol., 106:210-218). These figures are consistent with clinical observations of prostate carcinomas, which normally exhibit a slow and lingering course of progression. Such disease progression results in relatively few prostate tumors developing into cases of clinical concern during the lifetime of the patient. If, upon detection with available methods, the cancer appears well-differentiated, organ-confined and focal, treatment normally can not extend the life expectancy of older patients.

Unfortunately, the prostate carcinomas that are progressive in nature frequently have already metastasized by the time of clinical detection with available methods. Survival rates for individuals with metastatic prostate cancer are quite low. Between these two extremes are patients with prostate tumors that will metastasize during their lifetimes, but have not yet done so. For these patients, surgical removal of the prostate is curative and extends life expectancy. Therefore, accurate determination of which group a newly diagnosed patient falls into is critical in determining optimal treatment and patient survival.

Currently there is at least one early and noninvasive test available to the physician for detecting asymptomatic disease. The presence of Prostate Specific Antigen (PSA) can be measured with relative ease from blood samples using standard antibody-based detection kits. Abnormally high levels of this antigen in a patient's serum indicate a likelihood of prostate disease, possibly either a carcinoma, Benign Prostatic Hyperplasia (BPH) or prostatitis. In the majority of cases, PSA elevation is due to BPH or prostatitis rather than carcinoma.

Although clinical and pathologic stage and histological grading systems (e.g., Gleason's) have been used to indicate prognosis for groups of patients based on the degree of tumor differentiation or the type of glandular pattern (Carter and Coffey, In: J. P. Karr and H. Yamanak (eds.), Prostate Cancer: The Second Tokyo Symposium, pp. 19-27, New York: Elsevier, 1989.; Diamond et al., J. Urol., 128: 729-734, 1982), these systems do not adequately predict the progression rate of the cancer. While the use of computer-system image analysis of histologic sections of primary lesions for “nuclear roundness” has been suggested as an aide in the management of individual patients (Diamond et al., 1982, J. Urol., 128:729-734), this method is of limited use in studying the progression of the disease.

The analysis of DNA content/ploidy using flow cytometry and FISH has been demonstrated to have utility predicting prostate cancer aggressiveness (Pearsons et al., 1993, J. Urol., 150:120-125; Macoska et al., 1994, Cancer Res., 54: 3824-3830; Visakorpi et al., 1994, Am. J. Pathol., 145:1-7; Takahashi et al., 1994, Cancer Res., 54:3574-3579; Alcaraz et al., Cancer Res., 55:3998-4002, 1994), but these methods are expensive, time-consuming, and the latter methodology requires the construction of centromere-specific probes for analysis. There also exist specific nuclear matrix proteins whose expression has been reported to be associated with prostate cancer. However, these protein markers apparently do not distinguish between BPH and prostate cancer (Partin et al., 1993, Cancer Res., 53:744-746). Unfortunately, markers that cannot distinguish between benign and malignant prostate tumors are of little value.

It would therefore be beneficial to provide specific methods and reagents for the diagnosis, staging, prognosis, monitoring, and treatment of diseases associated with prostate cancer, or to indicate a predisposition to such for preventative medicine.

### SUMMARY OF THE INVENTION

The invention relates to a method of assessing whether a patient is afflicted with prostate cancer. The method of the present invention comprises the step of comparing the level of expression of a marker (listed within Tables 1-1 to 6) in a patient sample with the normal level of expression of the marker in a control, e.g., a sample from a patient without prostate cancer. A significant difference between the level of expression of the marker in the patient sample and the normal level is an indication that the patient is afflicted with prostate cancer.

In one embodiment of the methods of the present invention, the sample comprises cells obtained from the patient. The cells may be found in a prostate tissue sample collected, for example, by a prostate tissue biopsy or histology section, or a bone marrow biopsy. In another embodiment, the patient sample is a prostate-associated body fluid. Such fluids include, for example, blood fluids, lymph, urine, prostatic fluid and semen.

In accordance with the methods of the present invention, the presence and/or level of expression of the marker in a sample can be assessed, for example, by detecting the presence in the sample of:

a protein or protein fragment corresponding to the marker (e.g. using a reagent, such as an antibody, an antibody derivative, or an antibody fragment, which binds specifically with the protein or protein fragment)

a metabolite which is produced directly (i.e., catalyzed) or indirectly by a protein corresponding to the marker

a transcribed polynucleotide (e.g. an mRNA or a cDNA), or fragment thereof, having at least a portion with which the marker is substantially homologous (e.g. by contacting a mixture of transcribed polynucleotides obtained from the sample with a substrate having one or more of the markers listed within Tables 1-1 to 6 fixed thereto at selected positions)

a transcribed polynucleotide or fragment thereof, wherein the polynucleotide anneals with the marker under stringent hybridization conditions.

The methods of the present invention are useful for further diagnosing patients having an identified prostate mass or symptoms associated with prostate cancer, e.g. abnormally high levels of PSA. The methods of the present invention can further be of particular use with patients having an enhanced risk of developing prostate cancer (e.g., patients having a familial history of prostate cancer and patients identified as having a mutant oncogene). The methods of the present invention may further be of particular use in monitoring the efficacy of treatment of a prostate cancer patient (e.g. the efficacy of chemotherapy).

All cancers have staging schemes that are used to describe the degree to which the cancer has progressed. The TNM staging approach assigns the primary tumor (T) to one of four stages (and to additional substages within these categories) based on the size and location of the primary tumor within the prostate. A T1 designation indicates a microscopic tumor which cannot be detected by a digital rectal exam. A T2NO designation refers to a tumor palpable upon a digital rectal exam but are contained within the prostate capsule (local disease). In all forms of stage T3 disease the tumors have extended through the prostate capsule into the surrounding connective tissue or seminal vesicles. The T4 designation refers to tumors that have escaped from the prostate and can be found in the pelvic region. The N stage refers to whether the primary tumor has spread to the regional lymph nodes (pelvic lymph nodes). The M stage refers to whether the tumor cells have metastasized to distant sites.

The methods of the present invention may be performed using a plurality (e.g. 2, 3, 5, or 10 or more) of markers. According to a method involving a plurality of markers, the level of expression in the sample of each of a plurality of markers independently selected from the markers listed in Tables 1-1 to 6 is compared with the normal level of expression of each of the plurality of markers in samples of the same type obtained from control humans not afflicted with prostate cancer. A significantly altered level of expression in the sample of one or more of the markers listed in Tables 1-1 to 6, or some combination thereof, relative to that marker's corresponding normal levels, is an indication that the patient is afflicted with prostate cancer. The markers of Tables 1-1 to 6 may also be used in combination with known prostate cancer markers in the methods of the present invention, e.g. PSA analysis.

In a preferred method of assessing whether a patient is afflicted with prostate cancer (e.g., new detection (“screening”), detection of recurrence, reflex testing), the method comprises comparing:

a) the level of expression of a marker in a patient sample, wherein at least one marker is selected from the markers of Tables 1-1 to 6, and

b) the normal level of expression of the marker in a control non-prostate cancer sample.

A significant difference between the level of expression of the marker in the patient sample and the normal level is an indication that the patient is afflicted with prostate cancer.

The invention further relates to a method of assessing the efficacy of a therapy for inhibiting prostate cancer in a patient. This method comprises comparing:

a) expression of a marker in a first sample obtained from the patient prior to providing at least a portion of the therapy to the patient, wherein the marker is selected from the group consisting of the markers listed within Tables 1-1 to 6, and

b) expression of the marker in a second sample obtained from the patient following provision of the portion of the therapy.

A significant difference between the level of expression of the marker in the second sample, relative to the first sample, is an indication that the therapy is efficacious for inhibiting prostate cancer in the patient.

It will be appreciated that in this method the “therapy” may be any therapy for treating prostate cancer including, but not limited to, chemotherapy, immunotherapy, gene therapy, radiation therapy and surgical removal of tissue. Thus, the methods of the invention may be used to evaluate a patient before, during and after therapy, for example, to evaluate the reduction in tumor burden.

The present invention therefore further comprises a method for monitoring the progression of prostate cancer in a patient, the method comprising:

a) detecting in a patient sample at a first time point, the expression of a marker, wherein the marker is selected from the group consisting of the markers listed in Tables 1-1 to 6;

b) repeating step a) at a subsequent time point in time; and

c) comparing the level of expression detected in steps a) and b), and therefrom monitoring the progression of prostate cancer in the patient.

The present invention also includes a method for assessing the aggressiveness or indolence of prostate cancer (e.g., staging), the method comprising comparing:

a) the level of expression of a marker in a patient sample, wherein at least one marker is selected from the markers of Tables 1-1 to 6, and

b) the normal level of expression of the marker in a control sample.

A significant difference between the level of expression in the sample and the normal level is an indication that the cancer is aggressive or indolent.

The present invention further includes a method for determining whether prostate cancer has metastasized or is likely to metastasize in the future, the method comprising comparing:

a) the level of expression of a marker in a patent sample, wherein at least one marker is selected from the markers of Tables 1-1 to 6 and

b) the normal level (or non-metastatic level) of expression of the marker in a control sample.

A significant difference between the level of expression in the patient sample and the normal level (or non-metastatic level) is an indication that the prostate cancer has metastasized or is likely to metastasize in the future.

The invention also includes a method of selecting a composition for inhibiting prostate cancer in a patient. This method comprises the steps of:

a) obtaining a sample comprising cancer cells from the patient;

b) separately maintaining aliquots of the sample in the presence of a plurality of test compositions;

c) comparing expression of a marker listed within Tables 1-1 to 6 in each of the aliquots; and

d) selecting one of the test compositions which alters the level of expression of the marker in the aliquot containing that test composition, relative to other test compositions.

In addition, the invention includes a method of inhibiting prostate cancer in a patient. This method comprises the steps of:

a) obtaining a sample comprising cancer cells from the patient;

b) separately maintaining aliquots of the sample in the presence of a plurality of test compositions;

c) comparing expression of a marker listed within Tables 1-1 to 6 in each of the aliquots; and

d) administering to the patient at least one of the test compositions which alters the level of expression of the marker in the aliquot containing that test composition, relative to other test compositions.

The invention also includes a kit for assessing whether a patient is afflicted with prostate cancer. This kit comprises reagents for assessing expression of a marker listed within Tables 1-1 to 6.

In another aspect, the invention relates to a kit for assessing the suitability of each of a plurality of compounds for inhibiting a prostate cancer in a patient. The kit comprises a reagent for assessing expression of a marker listed within Tables 1-1 to 6, and may also comprise a plurality of compounds.

In another aspect, the invention relates to a kit for assessing the presence of prostate cancer cells. This kit comprises an antibody, wherein the antibody binds specifically with a protein or protein fragment corresponding to a marker listed within Tables 1-1 to 6. The kit may also comprise a plurality of antibodies, wherein the plurality binds specifically with a protein or protein fragment corresponding to a different marker listed within Tables 1-1 to 6.

The invention also includes a kit for assessing the presence of prostate cancer cells, wherein the kit comprises a nucleic acid probe. The probe binds specifically with a transcribed polynucleotide corresponding to a marker listed within Tables 1-1 to 6. The kit may also comprise a plurality of probes, wherein each of the probes binds specifically with a transcribed polynucleotide corresponding to a different marker listed within Tables 1-1 to 6.

The invention further relates to a method of making an isolated hybridoma which produces an antibody useful for assessing whether a patient is afflicted with prostate cancer. The method comprises isolating a protein or protein fragment corresponding to a marker listed within Tables 1-1 to 6, immunizing a mammal using the isolated protein or protein fragment, isolating splenocytes from the immunized mammal, fusing the isolated splenocytes with an immortalized cell line to form hybridomas, and screening individual hybridomas for production of an antibody which specifically binds with the protein or protein fragment, to isolate the hybridoma. The invention also includes an antibody produced by this method.

The invention further includes a method of assessing the prostate carcinogenic potential of a test compound. This method comprises the steps of:

a) maintaining separate aliquots of prostate cells in the presence and absence of the test compound; and

b) comparing expression of a marker in each of the aliquots.

The marker is selected from those listed within Tables 1-1 to 6. A significant difference between the level of expression of the marker in the aliquot maintained in the presence of (or exposed to) the test compound, relative to the aliquot maintained in the absence of the test compound, is an indication that the test compound possesses prostate carcinogenic potential.

Additionally, the invention includes a kit for assessing the prostate carcinogenic potential of a test compound. The kit comprises prostate cells and a reagent for assessing expression of a marker in each of the aliquots. The marker is selected from those listed within Tables 1-1 to 6.

The invention further relates to a method of treating a patient afflicted with prostate cancer. This method comprises providing to cells of the patient an antisense oligonucleotide complementary to a polynucleotide corresponding to a marker listed within Tables 1-1 to 6, which is overexpressed in prostate cancer. In an alternative method, expression of a gene corresponding to a marker selected from the markers listed in Tables 1-1 to 6 which is underexpressed in prostate cancer, is increased.

The invention includes a method of inhibiting prostate cancer in a patient at risk for developing prostate cancer. This method comprises inhibiting or increasing expression (or overexpression) of a gene corresponding to a marker listed within Tables 1-1 to 6, that is either overexpressed or underexpressed, respectively, in prostate cancer.

It will be appreciated that the methods and kits of the present invention may also include known cancer markers including known prostate cancer markers. It will further be appreciated that the methods and kits may be used to identify cancers other than prostate cancer.

### DETAILED DESCRIPTION OF THE INVENTION

The invention relates to newly discovered correlations between expression of certain markers and the cancerous state of prostate cells. It has been discovered that the level of expression of individual markers and combinations of markers described herein correlates with the presence of prostate cancer or a pre-malignant condition in a patient. Methods are provided for detecting the presence of prostate cancer in a sample, the absence of prostate cancer in a sample, the stage of a prostate cancer, the metastatic potential of a prostate cancer, the indolence or aggressiveness of the cancer, and other characteristics of prostate cancer that are relevant to prevention, diagnosis, characterization and therapy of prostate cancer in a patient.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

A “marker” is a naturally-occurring polymer corresponding to at least one of the nucleic acids listed within Tables 1-1 to 6. For example, markers include, without limitation, sense and anti-sense strands of genomic DNA (i.e. including any introns occurring therein), RNA generated by transcription of genomic DNA (i.e. prior to splicing), RNA generated by splicing of RNA transcribed from genomic DNA, and proteins generated by translation of spliced RNA (i.e. including proteins both before and after cleavage of normally cleaved regions such as transmembrane signal sequences). As used herein, “marker” may also include a cDNA made by reverse transcription of an RNA generated by transcription of genomic DNA (including spliced RNA).

As used herein a polynucleotide “corresponds to” another (a first) polynucleotide if it is related to the first polynucleotide by any of the following relationships: The second polynucleotide comprises the first polynucleotide and the second polynucleotide encodes a gene product; 2) The second polynucleotide is 5′ or 3′ to the first polynucleotide in cDNA, RNA, genomic DNA, or fragment of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide. The first and second polynucleotide may be fragments of a gene coding for a gene product. The second polynucleotide may be an exon of the gene while the first polynucleotide may be an intron of the gene; 3) The second polynucleotide is the complement of the first polynucleotide.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, cDNA, proteins, antibodies, and organic monomers.

A “prostate-associated” body fluid is a fluid which, when in the body of a patient, contacts or passes through prostate cells or into which cells or proteins shed from prostate cells are capable of passing. Exemplary prostate-associated body fluids include blood fluids, semen, prostate fluid, lymph and urine.

The “normal” level of expression of a marker is the level of expression of the marker in prostate cells or prostate-associated body fluids of a patient, e.g. a human, not afflicted with prostate cancer.

“Over-expression” and “under-expression” of a marker refer to expression of the marker of a patient at a greater or lesser level, respectively, than normal level of expression of the marker (e.g. at least two-fold greater or lesser level).

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “transcribed polynucleotide” is a polynucleotide (e.g. an RNA, a cDNA, or an analog of one of an RNA or cDNA) which is complementary to or homologous with all or a portion of a mature RNA made by transcription of a genomic DNA corresponding to a marker of the invention and normal post-transcriptional processing (e.g. splicing), if any, of the transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

A marker is “fixed” to a substrate if it is covalently or non-covalently associated with the substrate such that the substrate can be rinsed with a fluid (e.g. standard saline citrate, pH 7.4) without a substantial fraction of the marker dissociating from the substrate.

As used herein, a “naturally-occurring” nucleic acid molecule refers to an RNA or DNA molecule having a nucleotide sequence that occurs in nature.

Expression of a marker in a patient is “significantly” higher than the normal level of expression of a marker if the level of expression of the marker is greater than the normal level by an amount greater than the standard error of the assay employed to assess expression, and preferably at least twice, and more preferably three, four, five or ten times that amount. Alternately, expression of the marker in the patient can be considered “significantly” higher or lower than the normal level of expression if the level of expression is at least about two, and preferably at least about three, four, or five times, higher or lower, respectively, than the normal level of expression of the marker.

Prostate cancer is “inhibited” if at least one symptom of the cancer is alleviated, terminated, slowed, or prevented. As used herein, prostate cancer is also “inhibited” if recurrence or metastasis of the cancer is reduced, slowed, delayed, or prevented.

A kit is any manufacture (e.g. a package or container) comprising at least one reagent, e.g. a probe, for specifically detecting a marker of the invention, the manufacture being promoted, distributed, or sold as a unit for performing the methods of the present invention.

Description

The present invention is based, in part, on identification of markers which are differentially expressed in prostate cancer cells when compared with normal (i.e. non-cancerous) prostate cells. The markers of the invention correspond to DNA, RNA, and polypeptide molecules which can be detected in one or both of normal and cancerous prostate cells. The presence, absence, or level of expression of one or more of these markers in prostate cells is herein correlated with the cancerous state of the tissue. The invention thus includes compositions, kits, and methods for assessing the cancerous state of prostate cells (e.g. cells obtained from a human, cultured human cells, archived or preserved human cells and in vivo cells).

The compositions, kits, and methods of the invention have the following uses, among others:

1) assessing whether a patient is afflicted with prostate cancer;

2) assessing the stage of prostate cancer in a human patient;

3) assessing the grade of prostate cancer in a patient;

4) assessing the benign or malignant nature of prostate cancer in a patient;

5) assessing the metastatic potential of prostate cancer in a patient;

6) assessing the histological type of neoplasm (e.g. Adenocarcinoma) associated with prostate cancer in a patient;

7) assessing the indolent or aggressive nature of prostate cancer in a patient;

8) making an isolated hybridoma which produces an antibody useful for assessing whether a patient is afflicted with prostate cancer;

9) assessing the presence of prostate cancer cells;

10) assessing the efficacy of one or more test compounds for inhibiting prostate cancer in a patient;

11) assessing the efficacy of a therapy for inhibiting prostate cancer in a patient;

12) monitoring the progression of prostate cancer in a patient;

13) selecting a composition or therapy for inhibiting prostate cancer in a patient;

14) treating a patient afflicted with prostate cancer;

15) inhibiting prostate cancer in a patient;

16) assessing the prostate carcinogenic potential of a test compound; and

17) inhibiting prostate cancer in a patient at risk for developing prostate cancer.

The invention thus includes a method of assessing whether a patient is afflicted with prostate cancer which includes assessing whether the patient has pre-metastasized prostate cancer. This method comprises comparing the level of expression of a marker in a patient sample and the normal level of expression of the marker in a control, e.g., a non-prostate cancer sample. A significant difference between the level of expression of the marker in the patient sample and the normal level is an indication that the patient is afflicted with prostate cancer. The marker is selected from the group consisting of the markers listed within Tables 1-1 to 6. Although one or more molecules corresponding to the markers listed within Tables 1-1 to 6 may have been described by others, the significance of the level of expression of these markers with regard to the cancerous state of prostate cells has not previously been recognized.

The invention also encompasses polynucleotides which differ from that of the polynucleotides described herein, but which produce the same phenotypic effect, such as an allelic variant. These altered, but phenotypically equivalent polynucleotides are referred to as “equivalent nucleic acids.” This invention also encompasses polynucleotides characterized by changes in non-coding regions that do not alter the polypeptide produced therefrom when compared to the polynucleotide herein. This invention further encompasses polynucleotides, which hybridize to the polynucleotides of the subject invention under conditions of moderate or high stringency. Alternatively, the polynucleotides are at least 85%, or at least 90%, or more preferably, greater or equal to 95% identical as determined by a sequence alignment program when run under default parameters.

The following summarizes the Tables of the present invention:

Table 1-1 shows the sequences expressed at least five-fold higher in at least one of five prostate stage T2NO tumors, or at least three-fold higher in at least two of five prostate stage T2NO tumors, when compared with the average expression level in BPH tissue. The average expression level in BPH tissue is the mean of four specimens.

Table 1-2 shows the sequences expressed at least five-fold higher in at least one of five prostate stage T2NO tumors, or at least three-fold higher in at least two of five prostate stage T2NO tumors, when compared with the average expression level in normal prostate tissue. The average expression level in the normal prostate tissue is the mean of four specimens.

Table 1-3 is a merged list of Tables 1-1 and 1-2.

Table 1-4 shows sequences which meet at least one of the following six criteria:

Sequences expressed at least ten-fold higher in at least one of five prostate stage T2NO tumors, when compared with the average expression level in normal prostate tissue.

Sequences expressed at least five-fold higher in at least three of five prostate stage T2NO tumors, when compared with the average expression level in normal prostate tissue.

Sequences expressed at least three-fold higher in five of five prostate stage T2NO tumors, when compared with the average expression level in normal prostate tissue.

Sequences expressed at least ten-fold higher in at least one of five prostate stage T2NO tumors, when compared with the average expression level in BPH.

Sequences expressed at least five-fold higher in at least three of five prostate stage T2NO tumors, when compared with the average expression level in BPH.

Sequences expressed at least three-fold higher in five of five prostate stage T2NO tumors, when compared with the average expression level in BPH.

Table 1-5 shows sequences which meet at least one of the following three criteria:

Sequences found in Table 1-4 which show overexpression relative to both normal prostate and BPH.

Sequences found in Table 1-4 which show evidence of a prostate restricted tissue distribution. An estimate of the tissue distribution for specific ESTs was made through the use of sequence clustering software. Both the UniGene clustering of the dbEST database and Pangea clustering of sequence databases were used. The tissue of origin for each of the ESTs comprising a cluster was collected. Clusters were identified which met the criteria of at least five ESTs in the cluster, and with at least 75% of the ESTs from prostate libraries.

Sequences found in Table 1-4 which show evidence of a translation product with a membrane bound or secreted sub-cellular location based on the partition of the corresponding mRNA with membrane bound polysomes.

Table 2-1 shows sequences expressed at least three-fold higher in at least two of the six prostate stage T3NO tumors or at least five-fold higher in at least one of the six prostate stage T3NO tumors, when compared with the average expression levels of four BPH tissue specimens.

Table 2-2 shows sequences from Table 2-1 which are overexpressed by at least three-fold in three or more of six T3NO tumors or at least five-fold in two or more of six T3NO tumors, relative to BPH. Sequences that show restricted prostate tissue distribution or show evidence of a translational product with a membrane bound or secreted sub-cellular localization based on the partitioning of the corresponding mRNA with membrane bound polysomes, are preferred.

Table 2-3 shows sequences from Table 2-1 which are at least five-fold overexpressed in at least three of six T3NO tumors or at least ten-fold overexpressed in at least two of six T3NO tumors relative to BPH. Sequences that show restricted prostate tissue distribution or show evidence of a translational product with a membrane bound or secreted sub-cellular localization based on the partitioning of the corresponding mRNA with membrane bound polysomes, are preferred.

Tables 2-4, 2-5 and 2-6 show the expression levels for sequences in T2NO and T3NO tumors with poor clinical outcome. These are compared with the average expression levels of sequences from T2NO tumors with good clinical outcome. “Good clinical outcome” is defined as the patient remaining disease free for at least five years or more following surgery. “Poor clinical outcome” is defined as the patient suffering disease recurrence following surgery within a period of less than five years.

The sequences included in Table 2-4 are expressed at least three-fold higher in any one of four poor clinical outcome tumors compared to the mean expression in five good clinical outcome tumors.

Table 2-5 shows sequences from Table 2-4 which showed at least three-fold higher expression in at least two of four poor clinical outcome tumors compared to the average expression in five good clinical outcome tumors.

Table 2-6 shows sequences from Table 2-4 which showed at least five-fold higher expression in at least two of four poor clinical outcome tumors compared to the average expression in five good clinical outcome tumors. Some sequences in this Table show a restricted prostate distribution.

Tables 2-7, 2-8 and 2-9 show the expression levels for sequences in T2NO tumors with good clinical outcome. These are compared to the average expression levels of sequences from T2NO and T3NO tumors with poor clinical outcome.

Table 2-7 shows sequences which showed at least three-fold higher expression in at least one of five good clinical outcome tumors when compared with the average expression of four poor clinical outcome tumors.

Table 2-8 shows sequences from Table 2-7 which showed at least three-fold higher expression in at least two of five good clinical outcome tumors compared to the average expression in four poor clinical outcome tumors.

Table 2-9 shows sequences from Table 2-7 which showed at least five-fold higher expression in at least two of five good clinical outcome tumors compared to the average expression in four poor clinical outcome tumors. Some sequences in Table 2-9 also show a restricted prostate tissue distribution.

Tables 2-10, 2-11 and 2-12 show the expression levels for sequences in the prostate tumor cell lines DU145, LNCaP and PC3. These are compared to the expression levels of sequences from the normal prostate epithelial cell strain, PrEC.

Table 2-10 shows sequences expressed at least three-fold higher in three of three, five-fold higher in at least two of three, or ten-fold higher in at least one of three prostate cancer cell lines relative to the expression in the normal PrEC cell strain.

Table 2-11 shows sequences from Table 2-10 which are expressed at least five-fold higher in at least two of the prostate cancer cell lines relative to the expression in the normal PrEC cell strain.

Table 2-12 shows sequences from Table 2-10 which are expressed at least ten-fold higher in at least two of the prostate cancer cell lines relative to the expression in the normal PrEC cell strain.

Table 3-1 shows 7511 nucleotide sequences that were identified through subtracted library experiments described herein. Table 3-2 includes sequences for the markers of Table 3-1 which are found in non-public databases (e.g., PREPATNUC).

Table 3-3 shows 2543 nucleotide sequences that were identified through subtracted library experiments described herein. Table 3-4 includes sequences for the markers of Table 3-3 which are found in non-public databases (e.g., PREPATNUC).

Table 3-5 shows 5857 nucleotide sequences that encode secreted proteins and were identified through subtracted library experiments described herein. Table 3-6 includes sequences for the markers of Table 3-5 which are found in non-public databases (e.g., PREPATNUC).

Table 4-1 shows 7653 nucleotide sequences that were identified through subtracted library experiments described herein. Table 4-2 includes sequences for the markers of Table 4-1 which are found in non-public databases (e.g., PREPATNUC).

Tables 5-1 and 5-2 show 116 and 123 nucleotide sequences (respectively) that were identified through transcriptional profiling experiments described herein. The nucleotide sequences were expressed in androgen-independent prostate tumors at levels that are 10-fold or greater than the levels of the same nucleotide sequence in androgen-dependant prostate tumors. Comparisons were made at 0, 7, and 14 days of androgen deprivation (e.g., castration).

Table 6 shows the accession number (“Acc. No.”) and corresponding GenBank GI number (“GI Number”) for the markers of the present invention. One skilled in the art may thus obtain from the Tables of the invention, both GenBank accession number as well as the GenBank GI number for a marker of the present invention, thereby identifying the nucleotide and/or polypeptide sequence of that marker.

Any marker or combination of markers listed within Tables 1-1 to 6, as well as any known markers in combination with the markers set forth within Tables 1-1 to 6, may be used in the compositions, kits, and methods of the present invention. In general, it is preferable to use markers for which the difference between the level of expression of the marker in prostate cancer cells or prostate-associated body fluids and the level of expression of the same marker in normal prostate cells or prostate-associated body fluids is as great as possible. Although this difference can be as small as the limit of detection of the method for assessing expression of the marker, it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater.

It will be appreciated that patient samples containing prostate cells may be used in the methods of the present invention. In these embodiments, the level of expression of the marker can be assessed by assessing the amount (e.g. absolute amount or concentration) of the marker in a prostate cell sample, e.g., prostate tissue sample obtained from a patient. The cell sample can, of course, be subjected to a variety of well-known post-collection preparative and storage techniques (e.g. fixation, storage, freezing, lysis, homogenization, DNA or RNA extraction, ultrafiltration, concentration, evaporation, centrifugation, etc.) prior to assessing the amount of the marker in the sample.

It will also be appreciated that certain markers correspond to proteins which are secreted from prostate cells (i.e. one or both of normal and cancerous cells) to the extracellular space surrounding the cells. These markers are preferably used in certain embodiments of the compositions, kits, and methods of the invention, owing to the fact that the protein corresponding to each of these markers can be detected in a prostate-associated body fluid sample. In addition, preferred in vivo techniques for detection of a protein corresponding to a marker of the invention include introducing into a subject a labeled antibody directed against the protein. For example, the antibody can be labeled with a radioactive marker whose presence and location in a subject can be detected by standard imaging techniques.

Although not every marker corresponding to a secreted protein is indicated as such herein, it is a simple matter for the skilled artisan to determine whether any particular marker corresponds to a secreted protein. In order to make this determination, the protein corresponding to a marker is expressed in a test cell (e.g. a cell of a prostate cell line), extracellular fluid is collected, and the presence or absence of the protein in the extracellular fluid is assessed (e.g. using a labeled antibody which binds specifically with the protein).

The following is an example of a method which can be used to detect secretion of a protein corresponding to a marker of the invention. About 8×10⁵ 293T cells are incubated at 37° C. in wells containing growth medium (Dulbecco's modified Eagle's medium {DMEM} supplemented with 10% fetal bovine serum) under a 5% (v/v) CO₂, 95% air atmosphere to about 60-70% confluence. The cells are then transfected using a standard transfection mixture comprising 2 micrograms of DNA comprising an expression vector encoding the protein and 10 microliters of LipofectAMINE™ (GIBCO/BRL Catalog no. 18342-012) per well. The transfection mixture is maintained for about 5 hours, and then replaced with fresh growth medium and maintained in an air atmosphere. Each well is gently rinsed twice with DMEM which does not contain methionine or cysteine (DMEM-MC; ICN Catalog no. 16-424-54). About 1 milliliter of DMEM-MC and about 50 microcuries of Trans-³⁵S™ reagent (ICN Catalog no. 51006) are added to each well. The wells are maintained under the 5% CO₂ atmosphere described above and incubated at 37° C. for a selected period. Following incubation, 150 microliters of conditioned medium is removed and centrifuged to remove floating cells and debris. The presence of the protein in the supernatant is an indication that the protein is secreted.

Examples of prostate-associated body fluids include blood fluids (e.g. whole blood, blood serum, blood having platelets removed therefrom, lymph, urine, prostatic fluid and semen. Many prostate-associated body fluids (i.e. usually excluding urine) can have prostate cells therein, particularly when the prostate cells are cancerous, and, more particularly, when the prostate cancer is metastasizing. Cell-containing fluids which can contain prostate cancer cells include, but are not limited to, whole blood, blood having platelets removed therefrom, lymph, prostatic fluid, and semen. Thus, the compositions, kits, and methods of the invention can be used to detect expression of markers corresponding to proteins having at least one portion which is displayed on the surface of cells which express it. Although the proteins having at least one cell-surface portion are not set forth herein, it is a simple matter for the skilled artisan to determine whether the protein corresponding to any particular marker comprises a cell-surface protein. For example, immunological methods may be used to detect such proteins on whole cells, or well known computer-based sequence analysis methods (e.g. the SIGNALP program; Nielsen et al., 1997, Protein Engineering 10:1-6) may be used to predict the presence of at least one extracellular domain (i.e. including both secreted proteins and proteins having at least one cell-surface domain). Expression of a marker corresponding to a protein having at least one portion which is displayed on the surface of a cell which expresses it may be detected without necessarily lysing the cell (e.g. using a labeled antibody which binds specifically with a cell-surface domain of the protein).

Expression of a marker of the invention may be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In another preferred embodiment, expression of a marker is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a protein or protein fragment corresponding to the marker, such as the protein encoded by the open reading frame corresponding to the marker or such a protein which has undergone all or a portion of its normal post-translational modification.

In another preferred embodiment, expression of a marker is assessed by preparing mRNA/cDNA (i.e. a transcribed polynucleotide) from cells in a patient sample, and by hybridizing the mRNA/cDNA with a reference polynucleotide which is a complement of a polynucleotide comprising the marker, and fragments thereof. cDNA can, optionally, be amplified using any of a variety of polymerase chain reaction methods prior to hybridization with the reference polynucleotide. Expression of one or more markers can likewise be detected using quantitative PCR to assess the level of expression of the marker(s). Alternatively, any of the many known methods of detecting mutations or variants (e.g. single nucleotide polymorphisms, deletions, etc.) of a marker of the invention may be used to detect occurrence of a marker in a patient.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a marker of the invention. If polynucleotides complementary to or homologous with a marker of the invention are differentially detectable on the substrate (e.g. detectable using radioactivity, different chromophores or fluorophores), are fixed to different selected positions, then the levels of expression of a plurality of markers can be assessed simultaneously using a single substrate (e.g. a “gene chip” microarray of polynucleotides fixed at selected positions). When a method of assessing marker expression is used which involves hybridization of one nucleic acid with another, it is preferred that the hybridization be performed under stringent hybridization conditions.

Because the compositions, kits, and methods of the invention rely on detection of a difference in expression levels of one or more markers of the invention, it is preferable that the level of expression of the marker is significantly greater than the minimum detection limit of the method used to assess expression in at least one of normal prostate cells and cancerous prostate cells.

It is understood that by routine screening of additional patient samples using one or more of the markers of the invention, it will be realized that certain of the markers are over- or underexpressed in cancers of various types, including specific prostate cancers, as well as other cancers such as ovarian cancers. For example, it will be confirmed that some of the markers of the invention are over-expressed in most (i.e. 50% or more) or substantially all (i.e. 80% or more) of prostate cancer. Furthermore, it will be confirmed that certain of the markers of the invention are associated with prostate cancer of various stages.

It will be appreciated that as a greater number of patient samples are assessed for expression of the markers of the invention and the outcomes of the individual patients from whom the samples were obtained are correlated, it will also be confirmed that altered expression of certain of the markers of the invention are strongly correlated with malignant cancers and that altered expression of other markers of the invention are strongly correlated with benign tumors. The compositions, kits, and methods of the invention are thus useful for characterizing one or more of the stage, grade, histological type, metastatic potential, indolent vs. aggressive phenotype and benign/malignant nature of prostate cancer in patients.

When the compositions, kits, and methods of the invention are used for characterizing one or more of the stage, grade, histological type, metastatic potential, indolent vs. aggressive phenotype and benign/malignant nature of prostate cancer in a patient, it is preferred that the marker or panel of markers of the invention is selected such that a positive result is obtained in at least about 20%, and preferably at least about 40%, 60%, or 80%, and more preferably in substantially all patients afflicted with a prostate cancer of the corresponding stage, grade, histological type, metastatic potential, indolent vs. aggressive phenotype or benign/malignant nature. Preferably, the marker or panel of markers of the invention is selected such that a positive predictive value (PPV) of greater than about 10% is obtained for the general population.

When a plurality of markers of the invention are used in the compositions, kits, and methods of the invention, the level of expression of each marker in a patient sample can be compared with the normal level of expression of each of the plurality of markers in non-cancerous samples of the same type, either in a single reaction mixture (i.e. using reagents, such as different fluorescent probes, for each marker or a mixture of similiarly labeled probes to access a plurality of markers that are fixed to a single substrate at different positions) or in individual reaction mixtures corresponding to one or more of the markers. In one embodiment, a significantly enhanced level of expression of more than one of the plurality of markers in the sample, relative to the corresponding normal levels, is an indication that the patient is afflicted with prostate cancer. When a plurality of markers is used, it is preferred that 2, 3, 4, 5, 8, 10, 12, 15, 20, 30, or 50 or more individual markers be used, wherein fewer markers are preferred.

In order to maximize the sensitivity of the compositions, kits, and methods of the invention (i.e. by interference attributable to cells of non-prostate origin in a patient sample), it is preferable that the marker of the invention used therein be a marker which has a restricted tissue distribution, e.g., normally not expressed in non-prostate tissue.

Only a small number of markers are known to be associated with prostate cancers (e.g. PSA, PSMA, PAP, PCA3, PCTA-1, PSCA and STEAP). These markers are not, of course, included among the markers of the invention, although they may be used together with one or more markers of the invention in a panel of markers, for example. It is well known that certain types of genes, such as oncogenes, tumor suppressor genes, growth factor-like genes, protease-like genes, and protein kinase-like genes are often involved with development of cancers of various types. Thus, among the markers of the invention, use of those which correspond to proteins which resemble known proteins encoded by known oncogenes and tumor suppressor genes, and those which correspond to proteins which resemble growth factors, proteases, and protein kinases are preferred.

Known oncogenes and tumor suppressor genes include, for example, abl, abr, akT2NO, apc, bcl2α, bcl2β, bcl3, bcr, brcal, brca2, cbl, ccnd1, cdc42, cdk4, crk-II, csf1rlfms, dbl, dcc, dpc4/smad4, e-cad, e2f1/rbap, egfr/erbb-1, elk1, elk3, eph, erg, ets1, ets2, fer, fgr/src2, fli1 /ergb2, fos, fps/fes, fra1, fra2, fyn, hck, hek, her2/erbb-2/neu, her3/erbb-3, her4/erbb-4, hras1, hsT2NO, hst1, igfbp2, ink4a, ink4b, inT2NO/fgf3, jun, junb, jund, kip2, kit, kras2a, kras2b, lck, lyn mas, max, mcc, mdm2, met, mlh1, mmp10, mos, msh2, msh3, msh6, myb, myba, mybb, myc, mycl1, mycn, nf1, nf2, nme2, nras, p53, pdgfb, phb, pim1, pms1, pms2, ptc, pten, raf1, rap1a, rb1, rel, ret, ros1, ski, src1, tal1, tgfbr2, tgfb3, tgfbr3, thra1, thrb, tiam1, timp3, tjp1, tp53, trk, vav, vhl, vil2, waf1, wnt1, wnT2NO, wt1, and yes1 (Hesketh, 1997, In: The Oncogene and Tumour Suppressor Gene Facts Book, 2nd Ed., Academic Press; Fishel et al., 1994, Science 266:1403-1405).

Known growth factors include platelet-derived growth factor alpha, platelet-derived growth factor beta (simian sarcoma viral {v-sis} oncogene homolog), thrombopoietin (myeloproliferative leukemia virus oncogene ligand, megakaryocyte growth and development factor), erythropoietin, B cell growth factor, macrophage stimulating factor 1 (hepatocyte growth factor-like protein), hepatocyte growth factor (hepapoietin A), insulin-like growth factor 1 (somatomedia C), hepatoma-derived growth factor, amphiregulin (schwannoma-derived growth factor), bone morphogenetic proteins 1, 2, 3, 3 beta, and 4, bone morphogenetic protein 7 (osteogenic protein 1), bone morphogenetic protein 8 (osteogenic protein 2), connective tissue growth factor, connective tissue activation peptide 3, epidermal growth factor (EGF), teratocarcinoma-derived growth factor 1, endothelin, endothelin 2, endothelin 3, stromal cell-derived factor 1, vascular endothelial growth factor (VEGF), VEGF-B, VEGF-C, placental growth factor (vascular endothelial growth factor-related protein), transforming growth factor alpha, transforming growth factor beta 1 and its precursors, transforming growth factor beta 2 and its precursors, fibroblast growth factor 1 (acidic), fibroblast growth factor 2 (basic), fibroblast growth factor 5 and its precursors, fibroblast growth factor 6 and its precursors, fibroblast growth factor 7 (keratinocyte growth factor), fibroblast growth factor 8 (androgen-induced), fibroblast growth factor 9 (glia-activating factor), pleiotrophin (heparin binding growth factor 8, neurite growth-promoting factor 1), brain-derived neurotrophic factor, and recombinant glial growth factor 2.

Known proteases include interleukin-1 beta convertase and its precursors, Mch6 and its precursors, Mch2 isoform alpha, Mch4, Cpp32 isoform alpha, Lice2 gamma cysteine protease, Ich-1S, Ich-1L, Ich-2 and its precursors, TY protease, matrix metalloproteinase 1 (interstitial collagenase), matrix metalloproteinase 2 (gelatinase A, 72 kD gelatinase, 72 kD type IV collagenase), matrix metalloproteinase 7 (matrilysin), matrix metalloproteinase 8 (neutrophil collagenase), matrix metalloproteinase 12 (macrophage elastase), matrix metalloproteinase 13 (collagenase 3), metallopeptidase 1, cysteine-rich metalloprotease (disintegrin) and its precursors, subtilisin-like protease Pc8 and its precursors, chymotrypsin, snake venom-like protease, cathepsin 1, cathepsin D (lysosomal aspartyl protease), stromelysin, aminopeptidase N, plasminogen, tissue plasminogen activator, plasminogen activator inhibitor type II, and urokinase-type plasminogen activator.

Known protein kinases include DAP kinase, serine/threonine protein kinases NIK, PK428, Krs-2, SAK, and EMK, interferon-inducible double stranded RNA dependent protein kinase, FAST kinase, AIM1, IPL1-like midbody-associated protein kinase-1, NIMA-like protein kinase 1 (NLK1), the cyclin-dependent kinases (cdk1-10), checkpoint kinase Chk1, Nek3 protein kinase, BMK1 beta kinase, Clk1, Clk2, Clk3, extracellular signal-regulated kinases 1, 3, and 6, cdc28 protein kinase 1, cdc28 protein kinase 2, pLK, Myt1, c-Jun N-terminal kinase 2, Cam kinase 1, the MAP kinases, insulin-stimulated protein kinase 1, beta-adrenergic receptor kinase 2, ribosomal protein S6 kinase, kinase suppressor of ras-1 (KSR1), putative serine/threonine protein kinase Prk, PkB kinase, cAMP-dependent protein kinase, cGMP-dependent protein kinase, type II cGMP-dependent protein kinase, protein kinases Dyrk2, Dyrk3, and Dyrk4, Rho-associated coiled-coil containing protein kinase p160ROCK, protein tyrosine kinase t-Ror1, Ste20-related kinases, cell adhesion kinase beta, protein kinase 3, stress-activated protein kinase 4, protein kinase Zpk, serine kinase hPAK65, dual specificity mitogen-activated protein kinases 1 and 2, casein kinase I gamma 2, p21-activated protein kinase Pak1, lipid-activated protein kinase PRK2, focal adhesion kinase, dual-specificity tyrosine-phosphorylation regulated kinase, myosin light chain kinase, serine kinases SRPK2, TESK1, and VRK2, B lymphocyte serine/threonine protein kinase, stress-activated protein kinases JNK1 and JNK2, phosphorylase kinase, protein tyrosine kinase Tec, Jak2 kinase, protein kinase Ndr, MEK kinase 3, SHB adaptor protein (a Src homology 2 protein), agammaglobulinaemia protein-tyrosine kinase (Atk), protein kinase ATR, guanylate kinase 1, thrombopoeitin receptor and its precursors, DAG kinase epsilon, and kinases encoded by oncogenes or viral oncogenes such as v-fgr (Gardner-Rasheed), v-abl (Abelson murine leukemia viral oncogene homolog 1), v-arg (Abelson murine leukemia viral oncogene homolog, Abelson-related gene), v-fes and v-fps (feline sarcoma viral oncogene and Fujinami avian sarcoma viral oncogene homologs), proto-oncogene c-cot, oncogene pim-1, and oncogene mas1.

It is recognized that the compositions, kits, and methods of the invention will be of particular utility to patients having an enhanced risk of developing prostate cancer and their medical advisors. Patients recognized as having an enhanced risk of developing prostate cancer include, for example, patients having a familial history of prostate cancer, patients identified as having a mutant oncogene (i.e. at least one allele), and patients determined through any other established medical criteria to be at risk for cancer or other malignancy.

The level of expression of a marker in normal (i.e. non-cancerous) human prostate tissue can be assessed in a variety of ways. In one embodiment, this normal level of expression is assessed by assessing the level of expression of the marker in a portion of prostate cells which appears to be non-cancerous and by comparing this normal level of expression with the level of expression in a portion of the prostate cells which is suspected of being cancerous. For example, the normal level of expression of a marker may be assessed using a non-affected portion of the prostate and this normal level of expression may be compared with the level of expression of the same marker in an affected portion of the prostate. Alternately, and particularly as further information becomes available as a result of routine performance of the methods described herein, population-average values for normal expression of the markers of the invention may be used. In other embodiments, the ‘normal’ level of expression of a marker may be determined by assessing expression of the marker in a patient sample obtained from a non-cancer-afflicted patient, from a patient sample obtained from a patient before the suspected onset of prostate cancer in the patient, from archived patient samples, and the like.

The invention includes compositions, kits, and methods for assessing the presence of prostate cancer cells in a sample (e.g. an archived tissue sample or a sample obtained from a patient). These compositions, kits, and methods are substantially the same as those described above, except that, where necessary, the compositions, kits, and methods are adapted for use with samples other than patient samples. For example, when the sample to be used is a parafinized, archived human tissue sample, it can be necessary to adjust the ratio of compounds in the compositions of the invention, in the kits of the invention, or the methods used to assess levels of marker expression in the sample. Such methods are well known in the art and within the skill of the ordinary artisan.

The invention includes a kit for assessing the presence of prostate cancer cells (e.g. in a sample such as a patient sample). The kit comprises a plurality of reagents, each of which is capable of binding specifically with a nucleic acid or polypeptide corresponding to a marker of the invention. Suitable reagents for binding with a polypeptide corresponding to a marker of the invention include antibodies, antibody derivatives, antibody fragments, and the like. Suitable reagents for binding with a nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids (e.g. SSC buffer) suitable for annealing complementary nucleic acids or for binding an antibody with a protein with which it specifically binds, one or more sample compartments, an instructional material which describes performance of a method of the invention, a sample of normal prostate cells, a sample of prostate cancer cells, and the like.

The invention also includes a method of making an isolated hybridoma which produces an antibody useful for assessing whether a patient is afflicted with prostate cancer. In this method, a protein or protein fragment corresponding to a marker of the invention is isolated (e.g. by purification from a cell in which it is expressed or by transcription and translation of a nucleic acid encoding the protein in vivo or in vitro using known methods). A vertebrate, preferably a mammal such as a mouse, rat, rabbit, or sheep, is immunized using the isolated protein or protein fragment. The vertebrate may optionally (and preferably) be immunized at least one additional time with the isolated protein or protein fragment, so that the vertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or protein fragment. The invention also includes hybridomas made by this method and antibodies made using such hybridomas.

The invention also includes a method of assessing the efficacy of a test compound for inhibiting prostate cancer cells. As described above, differences in the level of expression of the markers of the invention correlate with the cancerous state of prostate cells. Although it is recognized that changes in the levels of expression of certain of the markers of the invention likely result from the cancerous state of prostate cells, it is likewise recognized that changes in the levels of expression of other of the markers of the invention induce, maintain, and promote the cancerous state of those cells. Thus, compounds which inhibit prostate cancer in a patient will cause the level of expression of one or more of the markers of the invention to change to a level nearer the normal level of expression for that marker (i.e. the level of expression for the marker in non-cancerous prostate cells).

This method thus comprises comparing expression of a marker in a first prostate cell sample and maintained in the presence of the test compound and expression of the marker in a second prostate cell sample and maintained in the absence of the test compound. A significant altered level of expression of a marker listed within Tables 1-1 to 6 is an indication that the test compound inhibits prostate cancer. The prostate cell samples may, for example, be aliquots of a single sample of normal prostate cells obtained from a patient, pooled samples of normal prostate cells obtained from a patient, cells of a normal prostate cell line, aliquots of a single sample of prostate cancer cells obtained from a patient, pooled samples of prostate cancer cells obtained from a patient, cells of a prostate cancer cell line, or the like. In one embodiment, the samples are prostate cancer cells obtained from a patient and a plurality of compounds known to be effective for inhibiting various prostate cancers are tested in order to identify the compound which is likely to best inhibit the prostate cancer in the patient.

This method may likewise be used to assess the efficacy of a therapy for inhibiting prostate cancer in a patient. In this method, the level of expression of one or more markers of the invention in a pair of samples (one subjected to the therapy, the other not subjected to the therapy) is assessed. As with the method of assessing the efficacy of test compounds, if the therapy induces a significant alteration in the level of expression of a marker listed within Tables 1-1 to 6 then the therapy is efficacious for inhibiting prostate cancer. As above, if samples from a selected patient are used in this method, then alternative therapies can be assessed in vitro in order to select a therapy most likely to be efficacious for inhibiting prostate cancer in the patient.

As described herein, prostate cancer in patients is associated with an altered level of expression of one or more markers listed within Tables 1-1 to 6. While, as discussed above, some of these changes in expression level result from occurrence of the prostate cancer, others of these changes induce, maintain, and promote the cancerous state of prostate cancer cells. Thus, prostate cancer characterized by an altered the level of expression of one or more markers listed within Tables 1-1 to 6 can be controlled or suppressed by altering expression of those markers.

Expression of a marker listed within Tables 1-1 to 6 can be inhibited in a number of ways generally known in the art. For example, an antisense oligonucleotide can be provided to the prostate cancer cells in order to inhibit transcription, translation, or both, of the marker(s). Alternately, a polynucleotide encoding an antibody, an antibody derivative, or an antibody fragment, and operably linked with an appropriate promoter/regulator region, can be provided to the cell in order to generate intracellular antibodies which will inhibit the function or activity of the protein corresponding to the marker(s). Using the methods described herein, a variety of molecules, particularly including molecules sufficiently small that they are able to cross the cell membrane, can be screened in order to identify molecules which inhibit expression of the marker(s). The compound so identified can be provided to the patient in order to inhibit expression of the marker(s) in the prostate cancer cells of the patient.

Expression of a marker listed in within Tables 1-1 to 6 can be enhanced in a number of ways generally known in the art. For example, a polynucleotide encoding the marker and operably linked with an appropriate promoter/regulator region can be provided to prostate cancer cells of the patient in order to induce enhanced expression of the protein (and mRNA) corresponding to the marker therein. Alternatively, if the protein is capable of crossing the cell membrane, inserting itself in the cell membrane, or is normally a secreted protein, then expression of the protein can be enhanced by providing the protein (e.g. directly or by way of the bloodstream or another prostate-associated fluid) to prostate cancer cells in the patient.

As described above, the cancerous state of human prostate cells is correlated with changes in the levels of expression of the markers of the invention. Thus, compounds which alter expression of one or more of the markers listed in within Tables 1-1 to 6 can induce prostate cell carcinogenesis. The invention thus includes a method for assessing the human prostate cell carcinogenic potential of a test compound. This method comprises maintaining separate aliquots of human prostate cells in the presence and absence of the test compound. Expression of a marker of the invention in each of the aliquots is compared. A significant alteration in the level of expression of a marker listed within Tables 1-1 to 6 in the aliquot maintained in the presence of the test compound (relative to the aliquot maintained in the absence of the test compound) is an indication that the test compound possesses human prostate cell carcinogenic potential. The relative carcinogenic potentials of various test compounds can be assessed by comparing the degree of enhancement or inhibition of the level of expression of the relevant markers, by comparing the number of markers for which the level of expression is enhanced or inhibited, or by comparing both.

Various aspects of the invention are described in further detail in the following subsections.

I. Isolated Nucleic Acid Molecules

One aspect of the invention pertains to isolated nucleic acid molecules that correspond to a marker of the invention, including nucleic acids which encode a polypeptide corresponding to a marker of the invention or a portion of such a polypeptide. Isolated nucleic acids of the invention also include nucleic acid molecules sufficient for use as hybridization probes to identify nucleic acid molecules that correspond to a marker of the invention, including nucleic acids which encode a polypeptide corresponding to a marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid encoding a protein corresponding to a marker listed in one or more of Tables 1-1 to 6, can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A process for identifying a larger fragment or the full-length coding sequence of a marker of the present invention is thus also provided. Any conventional recombinant DNA techniques applicable for isolating polynucleotides may be employed. One such method involves the 5′-RACE-PCR technique, in which the poly-A mRNA that contains the coding sequence of particular interest is first reverse transcribed with a 3′-primer comprising a sequence disclosed herein. The newly synthesized cDNA strand is then tagged with an anchor primer with a known sequence, which preferably contains a convenient cloning restriction site attached at the 5′ end. The tagged cDNA is then amplified with the 3′-primer (or a nested primer sharing sequence homology to the internal sequences of the coding region) and the 5′-anchor primer. The amplification may be conducted under conditions of various levels of stringency to optimize the amplification specificity. 5′-RACE-PCR can be readily performed using commercial kits (available from, e.g., BRL Life Technologies Inc., Clotech) according to the manufacturer's instructions.

Isolating the complete coding sequence of a gene can also be carried out in a hybridization assay using a suitable probe. The probe preferably comprises at least 10 nucleotides, and more preferably exhibits sequence homology to the polynucleotides of the markers of the present invention. Other high throughput screens for cDNAs, such as those involving gene chip technology, can also be employed in obtaining the complete cDNA sequence.

In addition, databases exist that reduce the complexity of ESTs by assembling contiguous EST sequences into tentative genes. For example, TIGR has assembled human ESTs into a database called THC for tentative human consensus sequences. The THC database allows for a more definitive assignment compared to ESTs alone. Software programs exist (TIGR assembler and TIGEM EST assembly machine and contig assembly program (see Huang, X., 1996, Genomes 33:21-23)) that allow for assembling ESTs into contiguous sequences from any organism.

Alternatively, mRNA from a sample preparation is used to construct cDNA library in the ZAP Express vector following the procedure described in Velculescu et al., 1997, Science 270:484. The ZAP Express cDNA synthesis kit (Stratagene) is used accordingly to the manufacturer's protocol. Plates containing 250 to 2000 plaques are hybridized as described in Rupert et al., 1988, Mol. Cell. Bio. 8:3104 to oligonucleotide probes with the same conditions previously described for standard probes except that the hybridization temperature is reduced to a room temperature. Washes are performed in 6×standard-saline-citrate 0.1% SDS for 30 minutes at room temperature. The probes are labeled with ³²P-ATP trough use of T4 polynucleotide kinase.

A partial cDNA (3′ fragment) can be isolated by 3′ directed PCR reaction. This procedure is a modification of the protocol described in Polyak et al., 1997, Nature 389:300. Briefly, the procedure uses SAGE tags in PCR reaction such that the resultant PCR product contains the SAGE tag of interest as well as additional cDNA, the length of which is defined by the position of the tag with respect to the 3′ end of the cDNA. The cDNA product derived from such a transcript driven PCR reaction can be used for many applications.

RNA from a source to express the cDNA corresponding to a given tag is first converted to double-stranded cDNA using any standard cDNA protocol. Similar conditions used to generate cDNA for SAGE library construction can be employed except that a modified oligo-dT primer is used to derive the first strand synthesis. For example, the oligonucleotide of composition 5′-B-TCC GGC GCG CCG TTT TCC CAG TCA CGA(30)-3′, contains a poly-T stretch at the 3′ end for hybridization and priming from poly-A tails, an M13 priming site for use in subsequent PCR steps, a 5′ Biotin label (B) for capture to strepavidin-coated magnetic beads, and an AscI restriction endonuclease site for releasing the cDNA from the strepavidin-coated magnetic beads. Theoretically, any sufficiently-sized DNA region capable of hybridizing to a PCR primer can be used as well as any other 8 base pair recognizing endonuclease.

cDNA constructed utilizing this or similar modified oligo-dT primer is then processed as described in U.S. Pat. No. 5,695,937 up until adapter ligation where only one adapter is ligated to the cDNA pool. After adapter ligation, the cDNA is released from the streptavidin-coated magnetic beads and is then used as a template for cDNA amplification.

Various PCR protocols can be employed using PCR priming sites within the 3′ modified oligo-dT primer and the SAGE tag. The SAGE tag-derived PCR primer employed can be of varying length dictated by 5′ extension of the tag into the adaptor sequence. cDNA products are now available for a variety of applications.

This technique can be further modified by: (1) altering the length and/or content of the modified oligo-dT primer; (2) ligating adaptors other than that previously employed within the SAGE protocol; (3) performing PCR from template retained on the streptavidin-coated magnetic beads; and (4) priming first strand cDNA synthesis with non-oligo-dT based primers.

Gene trapper technology can also be used. The reagents and manufacturer's instructions for this technology are commercially available from Life Technologies, Inc., Gaithsburg, Md. Briefly, a complex population of single-stranded phagemid DNA containing directional cDNA inserts is enriched for the target sequence by hybridization in solution to a biotinylated oligonucleotide probe complementary to the target sequence. The hybrids are captured on streptavidin-coated paramagnetic beads. A magnet retrieves the paramagnetic beads from the solution, leaving nonhybridized single-stranded DNAs behind. Subsequently, the captured single-stranded DNA target is released from the biotinylated oligonucleotide. After release, the cDNA clone is further enriched by using a nonbiotinylated target oligonucleotide to specifically prime conversion of the single-stranded DNA. Following transformation and plating, typically 20% to 100% of the colonies represent the cDNA clone of interest. To identify the desired cDNA clone, the colonies may be screened by colony hybridization using the ³²P-labeled oligonucleotide, or alternatively by DNA sequencing and alignment of all sequences obtained from numerous clones to determine a consensus sequence.

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a nucleic acid corresponding to a marker of the invention or to the nucleotide sequence of a nucleic acid encoding a protein which corresponds to a marker of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker of the invention or which encodes a polypeptide corresponding to a marker of the invention. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acids encoding a protein which corresponds to a marker of the invention, and thus encode the same protein.

In addition to the nucleotide sequences described in the GenBank and IMAGE Consortium database records described herein, and in Table ______, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid corresponding to a marker of the invention or to a nucleic acid encoding a protein corresponding to a marker of the invention. As used herein, the term “hybridizes under stringent conditions” is intended to describe conditions for hybridization and washing under which nucleotide sequences at least 75% (80%, 85%, preferably 90%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions for annealing two single-stranded DNA each of which is at least about 100 bases in length and/or for annealing a single-stranded DNA and a single-stranded RNA each of which is at least about 100 bases in length, are hybridization in 6×sodium chloride/sodium citrate (SSC) at about 45° C., followed by one or more washes in 0.2×SSC, 0.1% SDS at 50-65° C. Further preferred hybridization conditions are taught in Lockhart, et al., Nature Biotechnology, Volume 14, 1996 August:1675-1680; Breslauer, et al., Proc. Natl. Acad. Sci. USA, Volume 83, 1986 June: 3746-3750; Van Ness, et al., Nucleic Acids Research, Volume 19, No. 19, 1991 September: 5143-5151; McGraw, et al., BioTechniques, Volume 8, No. 6 1990: 674-678; and Milner, et al., Nature Biotechnology, Volume 15, 1997 June: 537-541, all expressly incorporated by reference.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, such a protein has an amino acid sequence that is at least about 40% identical, 50%, 60%, 70%, 80%, 90%, 95%, or 98% identical to the amino acid sequence of one of the proteins which correspond to the markers of the invention.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded cDNA molecule corresponding to a marker of the invention or complementary to an mRNA sequence corresponding to a marker of the invention. Accordingly, an antisense nucleic acid of the invention can hydrogen bond to (i e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker of the invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a prostate-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual a-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acids having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

II. Isolated Proteins and Antibodies

One aspect of the invention pertains to isolated proteins which correspond to individual markers of the invention, and biologically active portions thereof, as well as polypeptide fragments suitable for use as immunogens to raise antibodies directed against a polypeptide corresponding to a marker of the invention. In one embodiment, the native polypeptide corresponding to a marker can be isolated from cells or tissue sources by an appropriate purification scheme using standard protein purification techniques. In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide corresponding to a marker of the invention include polypeptides comprising amino acid sequences sufficiently identical to or derived from the amino acid sequence of the protein corresponding to the marker (e.g., the amino acid sequence listed in the GenBank and IMAGE Consortium database records described herein), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence listed in the one of the GenBank and IMAGE Consortium database records described herein. Other useful proteins are substantially identical (e.g., at least about 40%, preferably 50%, 60%, 70%, 80%, 90%, 95%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a marker of the invention. As used herein, a “chimeric protein” or “fusion protein” comprises all or part (preferably a biologically active part) of a polypeptide corresponding to a marker of the invention operably linked to a heterologous polypeptide (i.e., a polypeptide other than the polypeptide corresponding to the marker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a marker of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide corresponding to a marker of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide corresponding to a marker of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the polypeptides corresponding to individual markers of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

An isolated polypeptide corresponding to a marker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides of the invention, and encompasses an epitope of the protein such that an antibody raised against the peptide forms a specific immune complex with a marker of the invention to which the protein corresponds. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. Alternatively, antibodies specific for a protein or polypeptide of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Antibodies of the invention may be used as therapeutic agents in treating cancers. In a preferred embodiment, completely human antibodies of the invention are used for therapeutic treatment of human cancer patients, particularly those having an ovarian cancer. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody directed against a polypeptide corresponding to a marker of the invention (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) can be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

Accordingly, in one aspect, the invention provides substantially purified antibodies or fragments thereof, and non-human antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45□ C. and washing in 0.2×SSC, 0.1% SDS at 65□ C. In various embodiments, the substantially purified antibodies of the invention, or fragments thereof, can be human, non-human, chimeric and/or humanized antibodies.

In another aspect, the invention provides non-human antibodies or fragments thereof, which antibodies or fragments specifically bind to a polypeptide comprising an amino acid sequence selected from the group consisting of: the amino acid sequence of the present invention, an amino acid sequence encoded by the cDNA of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45□ C. and washing in 0.2×SSC, 0.1% SDS at 65□ C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to an amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45□ C. and washing in 0.2×SSC, 0.1% SDS at 65□ C. The monoclonal antibodies can be human, humanized, chimeric and/or non-human antibodies.

The substantially purified antibodies or fragments thereof may specifically bind to a signal peptide, a secreted sequence, an extracellular domain, a transmembrane or a cytoplasmic domain or cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence or an extracellular domain of the amino acid sequences of the present invention.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that specifically recognizes a polypeptide of the present invention, the method comprising immunizing a, mammal with a polypeptide. The polypeptide used as an immungen comprises an amino acid sequence selected from the group consisting of the amino acid sequence of the present invention, an amino acid sequence encoded by the cDNA of the nucleic acid molecules of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45□ C. and washing in 0.2×SSC, 0.1% SDS at 65□ C.

After immunization, a sample is collected from the mammal that contains an antibody that specifically recognizes the polypeptide. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

III. Recombinant Expression Vectors and Host Cells

Another aspect of the invention pertains to vectors, preferably expression vectors, containing a nucleic acid encoding a polypeptide corresponding to a marker of the invention (or a portion of such a polypeptide). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 1 85, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide corresponding to a marker of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol.185, Academic Press, San Diego, Calif., 1991). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, Nature 329:840) and pMT2NOPC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., 1986, Trends in Genetics, Vol. 1(1).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide corresponding to a marker of the invention. Accordingly, the invention further provides methods for producing a polypeptide corresponding to a marker of the invention using the host cells of the invention. In one embodiment, the method comprises culturing the host cell of invention (into which a recombinant expression vector encoding a polypeptide of the invention has been introduced) in a suitable medium such that the marker is produced. In another embodiment, the method further comprises isolating the marker polypeptide from the medium or the host cell.

The host cells of the invention can also be used to produce nonhuman transgenic animals. For example, in one embodiment, a host cell of the invention is a fertilized oocyte or an embryonic stem cell into which a sequences encoding a polypeptide corresponding to a marker of the invention have been introduced. Such host cells can then be used to create non-human transgenic animals in which exogenous sequences encoding a marker protein of the invention have been introduced into their genome or homologous recombinant animals in which endogenous gene(s) encoding a polypeptide corresponding to a marker of the invention sequences have been altered. Such animals are useful for studying the function and/or activity of the polypeptide corresponding to the marker and for identifying and/or evaluating modulators of polypeptide-activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a nucleic acid encoding a polypeptide corresponding to a marker of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, 4,873,191 and in Hogan, Manipulating the Mouse Embryo, Cold Spring Harbor Laboratory Press, Cold Spring Harbor, N.Y., 1986. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide corresponding to a marker of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi, 1987, Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al., 1992, Cell 69:915). The selected cells are then injected into a blastocyst of an animal (e.g., a mouse) to form aggregation chimeras (see, e.g., Bradley, Teratocarcinomas and Embryonic Stem Cells. A Practical Approach, Robertson, Ed., IRL, Oxford, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al., 1991, Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

IV. Pharmaceutical Compositions

The nucleic acid molecules, polypeptides, and antibodies (also referred to herein as “active compounds”) corresponding to a marker of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid corresponding to a marker of the invention. Such methods comprise formulating a pharmaceutically acceptable carrier with an agent which modulates expression or activity of a polypeptide or nucleic acid corresponding to a marker of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a marker of the invention and one or more additional active compounds.

The invention also provides methods (also referred to herein as “screening assays”) for identifying modulators, i.e., candidate or test compounds or agents (e.g., peptides, peptidomimetics, peptoids, small molecules or other drugs) which (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, USP 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:30 L-310; Ladner, supra.).

In one embodiment, the invention provides assays for screening candidate or test compounds which are substrates of a marker or biologically active portion thereof. In another embodiment, the invention provides assays for screening candidate or test compounds which bind to a marker or biologically active portion thereof. Determining the ability of the test compound to directly bind to a marker can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the activity of a marker or a biologically active portion thereof. In all likelihood, the marker can, in vivo, interact with one or more molecules, such as but not limited to, peptides, proteins, hormones, cofactors and nucleic acids. For the purposes of this discussion, such cellular and extracellular molecules are referred to herein as “binding partners” or marker “substrate”.

One necessary embodiment of the invention in order to facilitate such screening is the use of the marker to identify its natural in vivo binding partners. There are many ways to accomplish this which are known to one skilled in the art. One example is the use of the marker protein as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al, 1993, Biotechniques 14:920-924; Iwabuchi et al, 1993 Oncogene 8:1693-1696; Brent WO94/10300) in order to identify other proteins which bind to or interact with the marker (binding partners) and, therefore, are possibly involved in the natural function of the marker. Such marker binding partners are also likely to be involved in the propagation of signals by the marker or downstream elements of a marker-mediated signaling pathway. Alternatively, such marker binding partners may also be found to be inhibitors of the marker.

The two-hybrid system is based on the modular nature of most transcription factors, which consist of separable DNA-binding and activation domains. Briefly, the assay utilizes two different DNA constructs. In one construct, the gene that encodes a marker protein fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a marker-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colonies containing the functional transcription factor can be isolated and used to obtain the cloned gene which encodes the protein which interacts with the marker protein.

In a further embodiment, assays may be devised through the use of the invention for the purpose of identifying compounds which modulate (e.g., affect either positively or negatively) interactions between a marker and its substrates and/or binding partners. Such compounds can include, but are not limited to, molecules such as antibodies, peptides, hormones, oligonucleotides, nucleic acids, and analogs thereof Such compounds may also be obtained from any available source, including systematic libraries of natural and/or synthetic compounds. The preferred assay components for use in this embodiment is an prostate cancer marker identified herein, the known binding partner and/or substrate of same, and the test compound. Test compounds can be supplied from any source.

The basic principle of the assay systems used to identify compounds that interfere with the interaction between the marker and its binding partner involves preparing a reaction mixture containing the marker and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the marker and its binding partner. Control reaction mixtures are incubated without the test compound or with a placebo. The formation of any complexes between the marker and its binding partner is then detected. The formation of a complex in the control reaction, but less or no such formation in the reaction mixture containing the test compound, indicates that the compound interferes with the interaction of the marker and its binding partner.

Conversely, the formation of more complex in the presence of compound than in the control reaction indicates that the compound may enhance interaction of the marker and its binding partner.

The assay for compounds that interfere with the interaction of the marker with its binding partner may be conducted in a heterogeneous or homogeneous format. Heterogeneous assays involve anchoring either the marker or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the markers and the binding partners (e.g., by competition) can be identified by conducting the reaction in the presence of the test substance, i.e., by adding the test substance to the reaction mixture prior to or simultaneously with the marker and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the marker or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the marker or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/marker fusion proteins or glutathione-S-transferaseibinding partner can be adsorbed onto glutathione sepharose beads (Sigma Chemical, St. Louis, Mo.) or glutathione derivatized microtiter plates, which are then combined with the test compound or the test compound and either the non-adsorbed marker or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of marker binding or activity determined using standard techniques.

Other techniques for immobilizing proteins on matrices can also be used in the screening assays of the invention. For example, either a marker or a marker binding partner can be immobilized utilizing conjugation of biotin and streptavidin. Biotinylated marker protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August;18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g. Heegaard, 1998, J. Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al. (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al. (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the marker and its binding partner.

Also within the scope of the present invention are methods for direct detection of interactions between the marker and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et at, U.S. Pat. No. 4,868,103). Generally, this technique involves the addition of a fluorophore label on a first ‘donor’ molecule (e.g., marker or test compound) such that its emitted fluorescent energy will be absorbed by a fluorescent label on a second, ‘acceptor’ molecule (e.g., marker or test compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A test substance which either enhances or hinders participation of one of the species in the preformed complex will result in the generation of a signal variant to that of background. In this way, test substances that modulate interactions between a marker and its binding partner can be identified in controlled assays.

In another embodiment, modulators of marker expression are identified in a method wherein a cell is contacted with a candidate compound and the expression of mRNA or protein, corresponding to a marker in the cell, is determined. The level of expression of mRNA or protein in the presence of the candidate compound is compared to the level of expression of mRNA or protein in the absence of the candidate compound. The candidate compound can then be identified as a modulator of marker expression based on this comparison. For example, when expression of marker mRNA or protein is greater (statistically significantly greater) in the presence of the candidate compound than in its absence, the candidate compound is identified as a stimulator of marker mRNA or protein expression. Conversely, when expression of marker mRNA or protein is less (statistically significantly less) in the presence of the candidate compound than in its absence, the candidate compound is identified as an inhibitor of marker mRNA or protein expression. The level of marker mRNA or protein expression in the cells can be determined by methods described herein for detecting marker mRNA or protein.

In another aspect, the invention pertains to a combination of two or more of the assays described herein. For example, a modulating agent can be identified using a cell-based or a cell free assay, and the ability of the agent to modulate the activity of a marker protein can be further confirmed in vivo, e.g., in a whole animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., an marker modulating agent, an antisense marker nucleic acid molecule, an marker-specific antibody, or an marker-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In, the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the prostate epithelium). A method for lipidation of antibodies is described by Cruikshank et al. (1997) J. Acquired Immune Deficiency Syndromes and Human Retrovirology 14:193.

The nucleic acid molecules corresponding to a marker of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Computer Readable Means and Arrays

Computer readable media comprising a marker(s) of the present invention is also provided. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a marker of the present invention.

As used herein, “recorded” refers to a process for storing information on computer readable medium. Those skilled in the art can readily adopt any of the presently known methods for recording information on computer readable medium to generate manufactures comprising the markers of the present invention.

A variety of data processor programs and formats can be used to store the marker information of the present invention on computer readable medium. For example, the nucleic acid sequence corresponding to the markers can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the markers of the present invention.

By providing the markers of the invention in computer readable form, one can routinely access the marker sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

The invention also includes an array comprising a marker(s) of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development and differentiation, tumor progression, progression of other diseases, in vitro processes, such a cellular transformation and senescence, autonomic neural and neurological processes, such as, for example, pain and appetite, and cognitive functions, such as learning or memory.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

VI. Predictive Medicine

The present invention pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining the level of expression of polypeptides or nucleic acids corresponding to one or more markers of the invention, in order to determine whether an individual is at risk of developing prostate cancer. Such assays can be used for prognostic or predictive purposes to thereby prophylactically treat an individual prior to the onset of the cancer.

Yet another aspect of the invention pertains to monitoring the influence of agents (e.g., drugs or other compounds administered either to inhibit prostate cancer or to treat or prevent any other disorder {i.e. in order to understand any prostate carcinogenic effects that such treatment may have}) on the expression or activity of a marker of the invention in clinical trials. These and other agents are described in further detail in the following sections.

A. Diagnostic Assays

An exemplary method for detecting the presence or absence of a polypeptide or nucleic acid corresponding to a marker of the invention in a biological sample involves obtaining a biological sample (e.g. a prostate smear) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a polypeptide corresponding to a marker of the invention include enzyme linked immunosorbent assays (ELISAs), Western blots, immunoprecipitations, immunohistochemistry and immunofluorescence. In vitro techniques for detection of genomic DNA include Southern hybridizations. Furthermore, in vivo techniques for detection of a polypeptide corresponding to a marker of the invention include introducing into a subject a labeled antibody directed against the polypeptide. For example, the antibody can be labeled with a radioactive marker whose presence and location in a subject can be detected by standard imaging techniques.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):14______; Hage, D. S., and Tweed, S. A. J Chromatogr B Biomed Sci Appl Oct. 10, 1997;699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from prostate cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al, 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the prostate cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-prostate cancer sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from prostate cancer or from non-prostate cancer cells of prostate tissue. The choice of the cell source is dependent on the use of the relative expression level. Using expression found in normal tissues as a mean expression score aids in validating whether the marker assayed is prostate specific (versus normal cells). In addition, as more data is accumulated, the mean expression value can be revised, providing improved relative expression values based on accumulated data. Expression data from prostate cells provides a means for grading the severity of the prostate cancer state.

In another embodiment of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from prostate cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis, immunohistochemistry and enzyme linked immunoabsorbant assay (ELISA). A skilled artisan can readily adapt known protein/antibody detection methods for use in determining whether prostate cells express a marker of the present invention.

In one format, antibodies, or antibody fragments, can be used in methods such as Western blots, immunohistochemistry or immunofluorescence techniques to detect the expressed proteins. In such uses, it is generally preferable to immobilize either the antibody, proteins, or cells containing proteins, on a solid support. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from prostate cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a polypeptide or nucleic acid corresponding to a marker of the invention in a biological sample (e.g. a prostate sample). Such kits can be used to determine if a subject is suffering from or is at increased risk of developing prostate cancer. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

B. Pharmacogenomics

Agents or modulators which have a stimulatory or inhibitory effect on expression of a marker of the invention can be administered to individuals to treat (prophylactically or therapeutically) prostate cancer in the patient. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the level of expression of a marker of the invention in an individual can be determined to thereby select appropriate agent(s) for therapeutic or prophylactic treatment of the individual.

Pharmacogenomics deals with clinically significant variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the level of expression of a marker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of expression of a marker of the invention.

This invention also provides a process for preparing a database comprising at least one of the markers set forth in Tables 1-1 to 6. For example, the polynucleotide sequences are stored in a digital storage medium such that a data processing system for standardized representation of the genes that identify a prostate cancer cell is compiled. The data processing system is useful to analyze gene expression between two cells by first selecting a cell suspected of being of a neoplastic phenotype or genotype and then isolating polynucleotides from the cell. The isolated polynucleotides are sequenced. The sequences from the sample are compared with the sequence(s) present in the database using homology search techniques. Greater than 90%, more preferably greater than 95% and more preferably, greater than or equal to 97% sequence identity between the test sequence and the polynucleotides of the present invention is a positive indication that the polynucleotide has been Isolated from a prostate cancer cell as defined above.

In an alternative embodiment, the polynucleotides of this invention are sequenced and the information regarding sequence and in some embodiments, relative expression, is stored in any functionally relevant program, e.g., in Compare Report using the SAGE software (available though Dr. Ken Kinzler at John Hopkins University). The Compare Report provides a tabulation of the polynucleotide sequences and their abundance for the samples normalized to a defined number of polynucleotides per library (say 25,000). This is then imported into MS-ACCESS either directly or via copying the data into an Excel spreadsheet first and then from there into MS-ACCESS for additional manipulations. Other programs such as SYBASE or Oracle that permit the comparison of polynucleotide numbers could be used as alternatives to MS-ACCESS. Enhancements to the software can be designed to incorporate these additional functions. These functions consist in standard Boolean, algebraic, and text search operations, applied in various combinations to reduce a large input set of polynucleotides to a manageable subset of a polynucleotide of specifically defined interest.

One skilled in the art may create groups containing one or more project(s) by combining the counts of specific polynucleotides within a group (e.g., GroupNormal=Normal1+Normal2, GroupTumor1+TumorCellLine). Additional characteristic values are also calculated for each tag in the group (e.g., average count, minimum count, maximum count). One skilled in the art may calculate individual tag count ratios between groups, for example the ratio of the average GroupNormal count to the average GroupTumor count for each polynucleotide. A statistical measure of the significance of observed differences in tag counts between groups may be calculated.

C. Monitoring Clinical Trials

Monitoring the influence of agents (e.g., drug compounds) on the level of expression of a marker of the invention can be applied not only in basic drug screening, but also in clinical trials. For example, the effectiveness of an agent to affect marker expression can be monitored in clinical trials of subjects receiving treatment for prostate cancer. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected markers of the invention in the pre-administration sample; (iii) obtaining one or more post-administration samples from the subject; (iv) detecting the level of expression of the marker(s) in the post-administration samples; (v) comparing the level of expression of the marker(s) in the pre-administration sample with the level of expression of the marker(s) in the post-administration sample or samples; and (vi) altering the administration of the agent to the subject accordingly. For example, increased administration of the agent can be desirable to increase expression of the marker(s) to higher levels than detected, i.e., to increase the effectiveness of the agent. Alternatively, decreased administration of the agent can be desirable to decrease expression of the marker(s) to lower levels than detected, i.e., to decrease the effectiveness of the agent.

D. Surrogate Markers

The markers of the invention may serve as surrogate markers for one or more disorders or disease states or for conditions leading up to disease states, and in particular, prostate cancer. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The markers of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker transcription or expression, the amplified marker may be in a quantity which is more readily detectable than the drug itself. Also, the marker may be more easily detected due to the nature of the marker itself; for example, using the methods described herein, antibodies may be employed in an immune-based detection system for a protein marker, or marker-specific radiolabeled probes may be used to detect a mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Pharm. 56 Suppl. 3: S16-S20.

The markers of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA or protein for specific tumor markers in a subject, a drug or course of treatment may be selected that is optimized for the treatment of the specific tumor likely to be present in the subject. Similarly, the presence or absence of a specific sequence mutation in marker DNA may correlate with drug response. The use of pharmacogenomic markers therefore permits the application of the most appropriate treatment for each subject without having to administer the therapy.

VII. Electronic Apparatus Readable Media and Arrays

Electronic apparatus readable media comprising a prostate cancer marker of the present invention is also provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the markers of the present invention.

A variety of software programs and formats can be used to store the marker information of the present invention on the electronic apparatus readable medium. For example, the nucleic acid sequence corresponding to the markers can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the markers of the present invention.

By providing the markers of the invention in readable form, one can routinely access the marker sequence information for a variety of purposes. For example, one skilled in the art can use the nucleotide or amino acid sequences of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has prostate cancer or a pre-disposition to prostate cancer, wherein the method comprises the steps of determining the presence or absence of a prostate cancer marker and based on the presence or absence of the prostate cancer marker, determining whether the subject has prostate cancer or a pre-disposition to prostate cancer and/or recommending a particular treatment for the prostate cancer or pre-prostate cancer condition.

The present invention further provides in an electronic system and/or in a network, a method for determining whether a subject has prostate cancer or a pre-disposition to prostate cancer associated with a prostate cancer marker wherein the method comprises the steps of determining the presence or absence of the prostate cancer marker, and based on the presence or absence of the prostate cancer marker, determining whether the subject has prostate cancer or a pre-disposition to prostate cancer, and/or recommending a particular treatment for the prostate cancer or pre-prostate cancer condition. The method may further comprise the step of receiving phenotypic information associated with the subject and/or acquiring from a network phenotypic information associated with the subject.

The present invention also provides in a network, a method for determining whether a subject has prostate cancer or a pre-disposition to prostate cancer associated with a prostate cancer marker, said method comprising the steps of receiving information associated with the prostate cancer marker receiving phenotypic information associated with the subject, acquiring information from the network corresponding to the prostate cancer marker and/or prostate cancer, and based on one or more of the phenotypic information, the prostate cancer marker, and the acquired information, determining whether the subject has prostate cancer or a pre-disposition to prostate cancer. The method may further comprise the step of recommending a particular treatment for the prostate cancer or pre-prostate cancer condition.

The present invention also provides a business method for determining whether a subject has prostate cancer or a pre-disposition to prostate cancer, said method comprising the steps of receiving information associated with the prostate cancer marker, receiving phenotypic information associated with the subject, acquiring information from the network corresponding to the prostate cancer marker and/or prostate cancer, and based on one or more of the phenotypic information, the prostate cancer marker, and the acquired information, determining whether the subject has prostate cancer or a pre-disposition to prostate cancer. The method may further comprise the step of recommending a particular treatment for the prostate cancer or pre-prostate cancer condition.

The invention also includes an array comprising a prostate cancer marker of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of prostate cancer, progression of prostate cancer, and processes, such a cellular transformation associated with prostate cancer.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

VIII. Experimental Protocol

A. Subtracted Libraries and Transcript Profiling

Subtracted libraries are generated using a PCR based method that allows the isolation of clones expressed at higher levels in one population of mRNA (tester) compared to another population (driver). Both tester and driver mRNA populations are converted into cDNA by reverse transcription, and then PCR amplified using the SMART PCR kit from Clontech. Tester and driver cDNAs are then hybridized using the PCR-Select cDNA subtraction kit from Clontech. This technique results in both subtraction and normalization, which is an equalization of copy number of low-abundance and high-abundance sequences. After generation of the subtractive libraries, a group of 96 or more clones from each library is tested to confirm differential expression by reverse Southern hybridization.

SEQ ID NOS: 1-7511 (of Table 3-1) were identified through the above-described subtractive library hybridization techniques. In Table 3-1, SEQ ID NOS: 1-1240 were from Library cMhqaa; SEQ ID NOS: 1241-2805 were from Library cMhqab; SEQ ID NOS: 2806-4723 were from Library cMhqac; SEQ ID NOS: 4724-5247 were from Library cMhqag; SEQ ID NOS: 5248-6897 were from Library cMhqad; and, SEQ ID NOS: 6898-7511 were from Library cMhqaf. The “tester” source for two of these subtracted libraries, cMhqaa and cMhqab, was comprised of cDNA generated from stage T3NO tumors. The “driver” source for the library-designated cMhqaa was cDNA prepared from benign prostate hyperplasia and activated lymphocytes [B cells, T cells (CD4 and CD8)]. The driver cDNA for the cMhqab library was prepared from benign prostate hyperplasia cDNA. The “tester” source for the cMhqac and cMhqag subtracted libraries was stage T2 and T3 cDNA derived from prostate cancer patients with poor clinical outcome, whose cancer had recurred following surgery. The “driver” source for these subtracted libraries (cMhqac and cMhqag) was activated lymphocytes and stage T2 tumor cDNA that was obtained from patients who had a good clinical outcome and their disease had not recurred after surgery. The cMhqad-subtracted library was prepared using stage T2 tumor cDNA from patients that had a good clinical outcome as tester. The driver source for this library (cMhqad) was obtained from activated lymphocytes and stage T2 and T3 tumor samples recovered from patients whose cancer had recurred following surgery. The tester for the cMhqaf library was cDNA obtained from the prostate cancer cell lines, DU145, LNCaP and PC3. The “driver” source for the cMhqaf-subtracted library was comprised of cDNA generated from a normal prostate cell strain, PrEC.

SEQ ID NOS: 7512-10054 (Table 3-3) were identified through the above-identified subtractive library hybridization techniques. These sequences were from Library cMhqae. The “tester” source for Library cMhqae was comprised of cDNA generated from metastatic prostate cancer cell lines (Du145, PC3 and LNCaP). The “driver” source was comprised of cDNA generated from a normal prostate cell strain, PrEC. Table 3-4 includes sequences for the markers of Tables 3-3 which are found in non-public databases (e.g., PREPATNUC).

SEQ ID NOS: 10055-15911 (Table 3-5) were identified through the above-identified subtractive library hybridization techniques. SEQ ID NOS: 10055-11680 were from Library cMhqah; SEQ ID NOS: 11681-13328 were from Library cMhqai; and SEQ ID NOS: 13329-15911 were from Library cMhqak. This “tester” DNA was synthesized from membrane-associated polysomes mRNA separated from other mRNA's (i.e., cytosolic, nuclear, etc.) by density sedimentation equilibrium. The “driver” source was cDNA prepared from the non-membrane bound fraction (free cytosolic polysomes) obtained from the same density sedimentation equilibrium experiment. This mRNA was shown to be depleted of both secreted and membrane bound protein by profiling on the same 20K array. Table 3-6 includes sequences for the markers of Table 3-5 which are found in non-public databases (e.g., PREPATNUC).

SEQ ID NOS: 1-7653 (of Table 4-1) were identified through the above-described subtractive library hybridization techniques. In Table 4-1, the listed markers can be found in the following databases:

| | |
|---|---|
| Sequences 1-1264 | dbEST |
| Sequences 1265-1912 | GenBank |
| Sequences 1949-1964 | PREPATNUC |
| Sequences 1965-1970 | dbEST |
| Sequences 1971-1973 | GenBank |
| Sequences 1974-1981 | dbEST |
| Sequences 1982-2011 | GenBank |
| Sequences 2012-3148 | dbEST |
| Sequences 3149-3687 | GenBank |
| Sequences 3688-3722 | NUCPATENT |
| Sequences 3723-3733 | PREPATNUC |
| Sequences 3734-3884 | GenBank |
| Sequences 3885-4128 | dbEST |
| Sequences 4129-4143 | NUCPATENT |
| Sequences 4144-4152 | PREPATNUC |
| Sequences 4153-4174 | GenBank |
| Sequences 4175-4176 | dbEST |
| Sequences 4177-4180 | GenBank |
| Sequences 4181-5009 | dbEST |
| Sequences 5010-5585 | GenBank |
| Sequences 5586-5613 | NUCPATENT |
| Sequence 5614 | PREPATNUC |
| Sequences 5615-5717 | GenBank |
| Sequences 5718-5858 | dbEST |
| Sequences 5859-5864 | NUCPATENT |
| Sequence 5865 | PREPATNUC |
| Sequences 5866-5881 | GenBank |
| Sequences 5882-7064 | dbEST |
| Sequences 7065-7573 | GenBank |
| Sequences 7574-7621 | NUCPATENT |
| Sequences 7622-7632 | PREPATNUC |
| Sequences 7633-7653 | GenBank. |

For transcript profiling, nylon arrays are prepared by spotting purified PCR product onto a nylon membrane using a robotic gridding system linked to a sample database. Several thousand clones are spotted on each nylon filter. RNA or DNA from clinical samples (tumor and normal), and cell lines as well as from subtracted libraries, are used for hybridization against the nylon arrays. The RNA or DNA is labeled utilizing an in vitro reverse transcription reaction that contains a radiolabeled nucleotide that is incorporated during the reaction. Alternatively, mRNA is converted into cDNA by reverse transcription, and then PCR amplified using the SMART PCR kit from Clontech. Hybridization experiments are carried out by combining labeled RNA or DNA samples with nylon filters in a hybridization chamber. Duplicate, independent hybridization experiments are performed to generate transcriptional profiling data. See, Nature Genetics, 21 (1999). Amplified cDNA is then radiolabelled using random priming with PRIME IT from Stratagene.

B. Proteomics

Proteins that are secreted by normal and transformed cells in culture are analyzed to identify those proteins that are likely to be secreted by cancerous cells into body fluids. Supernatants are isolated and MWT-CO filters are used to simplify the mixture of proteins. The proteins are then digested with trypsin. The tryptic peptides are loaded onto a microcapillary HPLC column where they are separated, and eluted directly into an ion trap mass spectrometer, through a custom-made electrospray ionization source. Throughout the gradient, sequence data is acquired through fragmentation of the four most intense ions (peptides) that elute off the column, while dynamically excluding those that have already been fragmented. In this way, approximately 2000 scans worth of sequence data are obtained, corresponding to approximately 50 to 200 different proteins in the sample. These data are searched against databases using correlation analysis tools, such as MS-Tag, to identify the proteins in the supernatants.

Protein profiling experiments were also undertaken to assess whether the proteins associated with the expression of individual markers of the invention are secreted. Transcriptional profiling experiments were performed on fractions of RNA that were obtained from either (a) endoplasmic reticulum-associated (ER-associated) ribosomes, or (b) free ribosomes. Eukaryotic RNA which is isolated from ER-associated ribosomes tends to encode secreted and membrane bound proteins rather than intracellular proteins. Accordingly, markers of the invention which exhibit significantly enhanced expression in fractions of RNA from ER-associated ribosomes (in comparison with RNA from free ribosomes) are predicted to be associated with secreted proteins.

C. Androgen Sensitivity Markers

A murine model was employed to generate prostate tumors from xenografts of human prostate tumor tissue (CWR22 model; see, e.g., Nagabhushan et al, (1996) Cancer Res. 56:3042-3046; Pretlow et al, (1993) J. Natl. Cancer Inst. 85:394-398). When the mice which carry such xenografts are castrated, the tumors generally regress due to androgen dependence. Some androgen-independent tumors will recur 3-10 months later (CWR22R tumors).

Transcriptional profiling was able to identify markers with significantly (ten-fold or greater) elevated expression in androgen-independent tumors (CWR22R) over androgen-dependent tumors (CWR22) under conditions of androgen presence (e.g. non-castrated animals) or androgen deprivation (e.g., 7 and 14 days post castration). Androgen-independent tumors are generally considered more clinically aggressive than androgen-dependant tumors.

VIII. Summary of the Data Provided in the Tables

The description for the fields of Tables 1-1 to 1-5 is listed below:

“Genbank Acc No.” corresponds to the GenBank accession number assigned the particular sequence (see, for example http://www.ncbi.nlm.nih.gov/Entrez/nucleotide.html). All referenced GenBank sequences are expressly incorporated herein by reference.

“Clone” corresponds to the cDNA clone number from the IMAGE Consortium (see, for example Lennon, G., et al., 1996, Genomics 33:151-152; and http://www-bio.llnl.gov/bbrp/image/image.htnl). All referenced IMAGE clone sequences are expressly incorporated herein by reference.

“Aver. of Tumors” corresponds to the average intensity of all the 5 stage T2NO tumors(MPM000115-119).

“Aver. of BPH” corresponds to the average intensity of the four BPH samples.

“MPM-115/aver BPH” corresponds to the fold over-expression of the tumor MPM000115 compared to the average BPH intensity.

“MPM-116/aver BPH” corresponds to the fold over-expression of the tumor MPM000116compared to the average BPH intensity.

“MPM-117/aver BPH” corresponds to the fold over-expression of the tumor MPM000117 compared to the average BPH intensity.

“MPM-118/aver BPH” corresponds to the fold over-expression of the tumor MPM-000118 compared to the average BPH intensity.

“MPM-119/aver BPH” corresponds to the fold over-expression of the tumor MPM000119 compared to the average BPH intensity.

“Tumor Aver./aver BPH” corresponds to the fold difference of the Aver. Of Tumors compared to the aver BPH.

“Aver of Normals” corresponds to the average intensity of the four Normal (non-tumor) tissue samples.

“MPM-115/aver Normal” corresponds to the fold over-expression of the tumor MPM000115 compared to the average Normal intensity.

“MPM-116/aver Normal” corresponds to the fold over-expression of the tumor MPM000116 compared to the average Normal intensity.

“MPM-117/aver Normal” corresponds to the fold over-expression of the tumor MPM000117 compared to the average Normal intensity.

“MPM-118/aver Normal” corresponds to the fold over-expression of the tumor MPM000118 compared to the average Normal intensity.

“MPM-119/aver Normal” corresponds to the fold over-expression of the tumor MPM000119 compared to the average Normal intensity.

“Avg Fold” corresponds to the fold difference of Aver of Tumors compared to aver of Normals.

“Tumor/Normal” corresponds to the fold change in average of Tumors compared to average of Normals.

“Tumor/BPH” corresponds to the fold change in average of Tumors compared to average of BPH.

“T/N 5×,>1/5” indicates whether the sequence is expressed 5 fold or greater in at least 1 tumor compared to its expression in normal prostate.

“T/N 3×,>2/5” indicates whether the sequence is expressed 3 fold or greater in at least 2 tumors compared to its expression in normal prostate.

“T/BPH 5×,>1/5” indicates whether the sequence is expressed 5 fold or greater in at least 1 tumor compared to its expression in BPH.

“T/BPH 3×,>2/5” indicates whether the sequence is expressed 3 fold or greater in at least 2 tumors compared to its expression in BPH.

“T/N 10×,>1/5” indicates whether the sequence is expressed 10 fold or greater in at least 1 tumor compared to its expression in normal prostate.

“T/N 5×,>3/5” indicates whether the sequence is expressed 5 fold or greater in at least 3 tumors compared to its expression in normal prostate.

“TIN 3×,>5/5” indicates whether the sequence is expressed 3 fold or greater in all 5 tumors compared to its expression in normal prostate.

“T/BPH 10×,>1/5” indicates whether the sequence is expressed 10 fold or greater in at least 1 tumor compared to its expression in BPH.

“T/BPH 5×,>3/5” indicates whether the sequence is expressed 5 fold or greater in at least 3 tumors compared to its expression in BPH.

“T/BPH 3×,>5/5” indicates whether the sequence is expressed 3 fold or greater in all 5 tumors compared to its expression in BPH.

“T/N & T/BPH” indicates whether the sequence is over-expressed in Tumors compared to both Normal and BPH tissues.

“Secreted” indicates whether the sequence is secreted or predicted to be secreted based on membrane-bound polysome experiments described herein.

“Prostate Spec Exp” indicates whether this sequence has prostate-specific expression based on the occurrence of this sequence in databases.

Listed below are the definitions of the abbreviations used in Tables 2-1 to 2-12:

“Ave. BPH” indicates the mean expression in four non-cancerous benign prostate hyperplasia (BPH) tissues designated MPM000180, MPM000181, MPM000182 and MP000183.

“Ave. Tumor” refers to the mean expression of six stage T3NO tumors designated MPM000120, MPM000121, MPM000122, MPM000123, MPM000124, MPM000125.

“Ave. Fold” refers to the fold increase in expression in tumor tissues relative that of BPH tissues (calculated as Ave. Tumor/Ave. BPH).

“Fold (MPM120/Ave. BPH)” refers to the fold increase in expression in prostate tumor MPM000120 over that observed in BPH tissue (calculated as expression score in MPM000120/Ave. BPH).

“Fold (MPM121/Ave. BPH)” refers to the fold increase in expression in prostate tumor MPM000121 over that observed in BPH tissue (calculated as expression score in MPM000121/Ave. BPH).

“Fold (MPM122/Ave. BPH)” refers to the fold increase in expression in prostate tumor MPM000122 over that observed in BPH tissue (calculated as expression score in MPM000122/Ave. BPH).

“Fold (MPM123/Ave. BPH)” refers to the fold increase in expression in prostate tumor MPM000123 over that observed in BPH tissue (calculated as expression score in MPM000123/Ave. BPH).

“Fold (MPM124/Ave. BPH)” refers to the fold increase in expression in prostate tumor MPM000124 over that observed in BPH tissue (calculated as expression score in MPM000124/Ave. BPH).

“Fold (MPM125/Ave. BPH)” refers to the fold increase in expression in prostate tumor MPM000125 over that observed in BPH tissue (calculated as expression score in MPM000125/Ave. BPH).

“Ave. Good” indicates the mean expression of five cancerous prostate tumors from five patients that were disease free for at least five years or more following surgery. These were all stage T2NO tumors designated as MPM000027, MPM000028, MPM000029, MPM000030 and MPM000035.

“Ave. Poor” refers to the mean expression of four prostate tumor tissues from four patients whose disease recurred following surgery within a period of less than 5 years. These were two stage T2NO and 2 stage T3NO tumors. These were designated as MPM000031, MPM000032, MPM000033 and MPM000034.

“Fold (Ave.Poor/Ave.Good)” refers to the fold increase in mean expression of tumor tissues from patients with poor clinical outcome relative to the mean expression of tumor tissues from patients with good clinical outcome.

“Fold (MPM000031/Ave. Good)” refers to the fold increase in expression in prostate tumor MPM000031 over that observed as the mean of the five tumor samples from patients with a good clinical outcome (calculated as expression score in MPM000031/Ave.Good).

“Fold (MPM000032/Ave. Good)” refers to the fold increase in expression in prostate tumor MPM000032 over that observed as the mean of the five tumor samples from patients with a good clinical outcome (calculated as expression score in MPM000032/Ave.Good).

“Fold (MPM000033/Ave. Good)” refers to the fold increase in expression in prostate tumor MPM000033 over that observed as the mean of the five tumor samples from patients with a good clinical outcome (calculated as expression score in MPM000033/Ave.Good).

“Fold (MPM000034/Ave. Good)” refers to the fold increase in expression in prostate tumor MPM000034 over that observed as the mean of the five tumor samples from patients with a good clinical outcome (calculated as expression score in MPM000034/Ave.Good).

“Fold (Ave.Good/Ave.Poor)” refers to the fold increase in mean expression of tumor tissues from patients with good clinical outcome relative to the mean expression of tumor tissues from patients with poor clinical outcome.

“Fold (MPM000027/Ave. Poor)” refers to the fold increase in expression in prostate tumor MPM000027 over that observed as the mean of the four tumor samples from patients with a poor clinical outcome (calculated as expression score in MPM000027/Ave.Poor).

“Fold (MPM000028/Ave. Poor)” refers to the fold increase in expression in prostate tumor MPM000028 over that observed as the mean of the four tumor samples from patients with a poor clinical outcome (calculated as expression score in MPM000028/Ave.Poor).

“Fold (MPM000029/Ave. Poor)” refers to the fold increase in expression in prostate tumor MPM000029 over that observed as the mean of the four tumor samples from patients with a poor clinical outcome (calculated as expression score in MPM000029/Ave.Poor).

“Fold (MPM000030/Ave. Poor)” refers to the fold increase in expression in prostate tumor MPM000030 over that observed as the mean of the four tumor samples from patients with a poor clinical outcome (calculated as expression score in MPM000030/Ave.Poor).

“Fold (MPM000035/Ave. Poor)” refers to the fold increase in expression in prostate tumor MPM000035 over that observed as the mean of the four tumor samples from patients with a poor clinical outcome (calculated as expression score in MPM000035/Ave.Poor).

“Ave. Cancer Cell Line Values” refers to the mean expression of three prostate cancer cell lines, designated MPM000002 (LNCaP), MPM000271 (PC3) and MPM000272 (DU145).

MPM1 refers to the normal prostate epithelial cell strain (PrEC), designated MPM000001.

“Fold (Ave.Cell line/MPM1)” refers to the fold increase in mean expression of the three prostate cancer cell line relative to the expression in the normal prostate cell strain MPM000001 (PrEC).

“Fold (MPM0000002/MPM000001)” refers to the fold increase in expression in prostate cancer cell line MPM0000002 (LNCaP) over that observed in MPM000001 (PrEC) the normal cell strain (calculated as expression score in MPM0000002/MPM000001).

“Fold (MPM0000271/MPM000001)” refers to the fold increase in expression in prostate cancer cell line MPM0000271 (PC3) over that observed in MPM000001 (PrEC) the normal cell strain (calculated as expression score in MPM000271/MPM000001).

“Fold (MPM0000272/MPM000001)” refers to the fold increase in expression in prostate cancer cell line MPM0000272 (DU145) over that observed in MPM000001 (PrEC) the normal cell strain (calculated as expression score in MPM0000272/MPM000001).

The description of the fields for Tables 3-1, 3-3, 3-5, 4-1 and 6 is listed below:

“Sequence #” or “Sequence ID” corresponds to the SEQ ID NO. assigned for each nucleotide sequence listed.

“Accession #” or “Accession” or “Acc No” corresponds to the accession number assigned to the nucleotide sequence in the relevant database.

“Database” or “Database hit” refers to the relevant database where the nucleotide sequence may be found according to its accession number. Those databases which are public include GenBank, dbEST (a division of GenBank), and NUCPATENT (a GENESEQ database, available through Derwent). For examples, see http://www.ncbi.nlm.nih.gov/Entrez/nucleotide.html for GenBank and www.derwent.com for GENESEQ. “GI number” is the GI identification number assigned to the marker in the GenBank database (see supra). Nucleic acid sequences of markers from Tables 3-1 and 4-1 which are from non-public databases (e.g., PREPATNUC) are given in Tables 3-2 and 4-2, respectively. All referenced database sequences are expressly incorporated herein by reference.

The description of the fields for Tables 5-1 and 5-2 is listed below:

“Order” and “Clone” correspond to assigned reference numbers for each nucleotide sequence listed.

“GenBank Accession Number” corresponds to the accession number assigned to the nucleotide sequence in the GenBank database.

Other Embodiments

Those skilled in the art will recognize, or be able to ascertain using no more than routine experimentation, many equivalents to the specific embodiments of the invention described herein. Such equivalents are intended to be encompassed by the following claims.

All publications including journal references, patents and databases are expressly incorporated by reference.

TABLE 1-1

| | Aver. of | Aver. of | MPM-115/ | MPM-116/ | MPM-117/ | MPM-118/ | MPM-119 | Tumor Aver |
|---|---|---|---|---|---|---|---|---|
| Clone | Tumors | BPH | Aver. | Aver. BPH | Aver. | Aver. BPH | Aver. BPH | / |
| 788180 | 25.18 | 0.50 | 102.19 | 5.44 | 15.38 | 90.33 | 40.12 | 50.69 |
| 308682 | 8.99 | 0.27 | 26.49 | 39.49 | 22.07 | 29.80 | 46.70 | 32.91 |
| 646037 | 24.42 | 0.75 | 53.07 | 14.65 | 28.67 | 17.92 | 49.22 | 32.70 |
| 281003 | 128.77 | 4.90 | 33.20 | 39.14 | 7.16 | 31.28 | 20.52 | 26.26 |
| 1034473 | 33.61 | 1.35 | 44.07 | 2.71 | 18.38 | 21.16 | 38.51 | 24.97 |
| 284701 | 33.05 | 1.53 | 17.19 | 22.80 | 11.69 | 43.48 | 13.22 | 21.68 |
| 66336 | 1.67 | 0.10 | 26.22 | 29.17 | 26.08 | 1.00 | 1.00 | 16.70 |
| 281039 | 14.05 | 0.90 | 1.80 | 7.16 | 1.52 | 66.03 | 1.32 | 15.57 |
| 26736 | 9.52 | 0.62 | 18.76 | 4.16 | 3.08 | 32.76 | 18.11 | 15.37 |
| 784168 | 53.04 | 3.83 | 4.81 | 12.77 | 16.92 | 15.79 | 18.91 | 13.84 |
| 486035 | 24.75 | 1.86 | 10.82 | 21.22 | 9.17 | 10.98 | 14.41 | 13.32 |
| 593185 | 28.06 | 2.29 | 3.25 | 8.22 | 9.64 | 26.71 | 13.34 | 12.23 |
| 222157 | 8.80 | 0.73 | 19.15 | 11.68 | 10.10 | 4.30 | 15.47 | 12.14 |
| 35058 | 15.59 | 1.44 | 4.36 | 13.99 | 6.63 | 22.14 | 6.94 | 10.81 |
| 730739 | 36.49 | 3.45 | 9.24 | 8.26 | 21.62 | 7.46 | 6.29 | 10.57 |
| 1636908 | 6.58 | 0.63 | 7.98 | 11.69 | 11.24 | 15.43 | 5.88 | 10.44 |
| 292515 | 16.22 | 1.56 | 8.49 | 16.42 | 6.85 | 7.64 | 12.51 | 10.38 |
| 450049 | 18.37 | 1.80 | 8.59 | 6.15 | 7.59 | 5.29 | 23.45 | 10.21 |
| 666334 | 1.64 | 0.16 | 9.09 | 11.54 | 10.25 | 7.37 | 12.48 | 10.14 |
| 32160 | 26.80 | 2.77 | 8.40 | 9.26 | 11.15 | 8.26 | 11.38 | 9.69 |
| 26519 | 36.05 | 3.76 | 10.15 | 14.36 | 8.76 | 6.89 | 7.79 | 9.59 |
| 42415 | 6.86 | 0.72 | 3.28 | 4.71 | 11.07 | 1.12 | 27.26 | 9.49 |
| 50772 | 18.10 | 1.91 | 4.58 | 9.38 | 7.33 | 20.62 | 5.42 | 9.47 |
| 1412481 | 8.41 | 0.90 | 0.84 | 0.56 | 0.97 | 43.41 | 0.78 | 9.31 |
| 197525 | 9.52 | 1.06 | 4.67 | 14.11 | 7.12 | 11.49 | 7.69 | 9.01 |
| 490178 | 24.55 | 2.73 | 5.26 | 4.27 | 26.85 | 3.26 | 5.29 | 8.99 |
| 461522 | 1.21 | 0.14 | 9.17 | 6.01 | 9.25 | 18.26 | 1.00 | 8.74 |
| 746152 | 9.35 | 1.07 | 15.24 | 8.42 | 15.81 | 3.05 | 1.00 | 8.70 |
| 712950 | 11.53 | 1.34 | 1.98 | 3.65 | 7.29 | 28.45 | 1.55 | 8.58 |
| 814053 | 14.66 | 1.75 | 7.99 | 1.27 | 6.56 | 19.08 | 7.03 | 8.38 |
| 1610448 | 135.55 | 16.44 | 12.94 | 8.87 | 5.99 | 4.91 | 8.53 | 8.25 |
| 825857 | 4.27 | 0.52 | 7.93 | 7.95 | 8.48 | 9.68 | 6.97 | 8.20 |
| 595297 | 3.07 | 0.38 | 10.77 | 9.58 | 5.96 | 9.36 | 5.19 | 8.17 |
| 595695 | 3.58 | 0.44 | 8.22 | 5.78 | 8.47 | 8.52 | 9.80 | 8.16 |
| 488964 | 7.77 | 0.96 | 26.20 | 6.06 | 2.31 | 3.91 | 1.95 | 8.08 |
| 259374 | 10.51 | 1.31 | 7.23 | 5.29 | 2.68 | 15.21 | 9.79 | 8.04 |
| 282089 | 11.18 | 1.41 | 1.99 | 11.92 | 1.50 | 23.33 | 0.88 | 7.92 |
| 823940 | 22.39 | 2.83 | 7.23 | 4.80 | 21.23 | 2.51 | 3.77 | 7.91 |
| 447715 | 14.83 | 1.88 | 7.03 | 18.09 | 5.79 | 5.60 | 3.00 | 7.90 |
| 1048810 | 20.15 | 2.59 | 8.33 | 12.64 | 4.86 | 8.53 | 4.52 | 7.78 |
| 46166 | 13.13 | 1.71 | 1.53 | 17.28 | 9.10 | 8.31 | 2.20 | 7.69 |
| 460279 | 0.87 | 0.12 | 11.78 | 9.75 | 7.52 | 6.64 | 2.24 | 7.59 |
| 687297 | 12.18 | 1.63 | 8.76 | 8.28 | 8.19 | 5.22 | 6.95 | 7.48 |
| 757392 | 0.86 | 0.12 | 8.05 | 9.45 | 7.56 | 8.53 | 3.46 | 7.41 |
| 731290 | 11.60 | 1.57 | 3.39 | 6.38 | 4.24 | 21.92 | 1.10 | 7.40 |
| 1556526 | 0.88 | 0.12 | 1.08 | 2.49 | 1.41 | 30.43 | 1.24 | 7.33 |
| 826109 | 0.75 | 0.10 | 9.78 | 6.45 | 6.11 | 10.44 | 3.60 | 7.27 |
| 825736 | 0.92 | 0.13 | 8.65 | 8.09 | 8.56 | 8.95 | 2.04 | 7.26 |
| 22161 | 20.24 | 2.80 | 5.54 | 5.05 | 9.71 | 9.20 | 6.71 | 7.24 |
| 283173 | 17.85 | 2.47 | 17.57 | 3.98 | 4.39 | 7.32 | 2.81 | 7.21 |
| 825822 | 14.07 | 1.97 | 5.87 | 8.14 | 6.53 | 10.54 | 4.71 | 7.16 |
| 280217 | 20.52 | 2.89 | 9.65 | 0.93 | 2.71 | 20.73 | 1.45 | 7.09 |
| 262053 | 10.75 | 1.52 | 5.67 | 9.07 | 8.71 | 8.34 | 3.60 | 7.08 |
| 66317 | 5.50 | 0.78 | 15.41 | 4.56 | 4.00 | 10.34 | 0.99 | 7.06 |
| 565235 | 35.83 | 5.15 | 3.19 | 8.34 | 3.79 | 17.38 | 2.08 | 6.96 |
| 1468260 | 4.53 | 0.65 | 13.06 | 1.36 | 7.45 | 4.03 | 8.89 | 6.96 |
| 826103 | 1.31 | 0.19 | 8.19 | 7.96 | 5.94 | 9.88 | 2.70 | 6.93 |
| 796366 | 34.94 | 5.05 | 10.57 | 5.34 | 12.35 | 3.13 | 3.18 | 6.92 |
| 877835 | 23.06 | 3.34 | 9.71 | 3.97 | 5.61 | 7.41 | 7.82 | 6.90 |
| 258300 | 18.15 | 2.65 | 4.31 | 7.13 | 13.32 | 1.26 | 8.27 | 6.86 |
| 814062 | 0.81 | 0.12 | 10.04 | 5.59 | 8.22 | 8.85 | 1.42 | 6.83 |
| 1034738 | 22.40 | 3.29 | 5.92 | 5.75 | 6.61 | 9.70 | 6.12 | 6.82 |
| 951313 | 6.84 | 1.01 | 2.78 | 4.19 | 15.48 | 7.44 | 3.86 | 6.75 |
| 253132 | 1.40 | 0.21 | 8.70 | 9.55 | 4.09 | 5.72 | 5.23 | 6.66 |
| 824510 | 1.38 | 0.21 | 8.56 | 9.50 | 7.92 | 5.67 | 1.59 | 6.65 |
| 826072 | 1.20 | 0.18 | 7.01 | 8.64 | 4.02 | 10.85 | 2.42 | 6.59 |
| 796754 | 0.66 | 0.10 | 6.20 | 5.19 | 5.58 | 9.09 | 6.47 | 6.50 |
| 251806 | 2.94 | 0.46 | 6.74 | 6.71 | 5.58 | 9.16 | 3.74 | 6.39 |
| 840306 | 2.49 | 0.39 | 2.46 | 5.82 | 8.43 | 9.48 | 5.73 | 6.38 |
| 815163 | 1.20 | 0.19 | 6.07 | 6.53 | 9.54 | 7.44 | 2.20 | 6.36 |
| 838285 | 1.34 | 0.21 | 4.71 | 7.31 | 6.15 | 8.05 | 5.54 | 6.35 |
| 565863 | 3.93 | 0.63 | 13.52 | 9.05 | 2.58 | 3.19 | 3.01 | 6.27 |
| 757435 | 365.78 | 58.35 | 3.90 | 3.17 | 17.54 | 3.19 | 3.55 | 6.27 |
| 67401 | 1.47 | 0.23 | 1.08 | 1.27 | 26.14 | 1.90 | 0.96 | 6.27 |
| 796239 | 26.07 | 4.18 | 1.88 | 2.16 | 10.01 | 7.23 | 9.94 | 6.24 |
| 321706 | 7.80 | 1.25 | 8.36 | 1.45 | 14.65 | 1.76 | 4.95 | 6.23 |
| 564801 | 12.35 | 1.99 | 5.89 | 2.75 | 17.66 | 3.44 | 1.34 | 6.21 |
| 950778 | 3.00 | 0.49 | 12.47 | 7.19 | 4.14 | 3.62 | 3.50 | 6.18 |
| 884606 | 33.46 | 5.43 | 6.01 | 3.92 | 16.90 | 2.25 | 1.74 | 6.16 |
| 454317 | 20.56 | 3.34 | 3.52 | 4.38 | 7.94 | 12.41 | 2.55 | 6.16 |
| 796646 | 6.21 | 1.01 | 5.13 | 3.61 | 5.34 | 6.22 | 10.48 | 6.16 |
| 840894 | 34.81 | 5.66 | 2.34 | 6.76 | 5.31 | 9.85 | 6.47 | 6.15 |
| 52933 | 3.59 | 0.58 | 5.96 | 6.27 | 4.93 | 6.70 | 6.87 | 6.14 |
| 594994 | 14.61 | 2.38 | 5.57 | 4.29 | 8.11 | 6.75 | 5.98 | 6.14 |
| 586780 | 1.92 | 0.31 | 11.12 | 7.73 | 4.39 | 3.68 | 3.68 | 6.12 |
| 868282 | 8.99 | 1.47 | 5.69 | 6.02 | 6.96 | 6.08 | 5.84 | 6.12 |
| 1535596 | 401.67 | 66.91 | 5.47 | 9.25 | 6.43 | 4.48 | 4.39 | 6.00 |
| 259973 | 1.66 | 0.28 | 6.61 | 6.24 | 5.06 | 6.62 | 5.42 | 5.99 |
| 839081 | 16.60 | 2.78 | 10.16 | 10.63 | 1.88 | 5.07 | 2.14 | 5.98 |
| 359661 | 26.24 | 4.39 | 4.04 | 9.90 | 3.64 | 7.90 | 4.38 | 5.97 |
| 509731 | 39.41 | 6.63 | 4.92 | 3.91 | 8.19 | 7.51 | 5.20 | 5.95 |
| 770066 | 11.43 | 1.93 | 1.49 | 2.31 | 5.43 | 18.27 | 2.17 | 5.94 |
| 151055 | 27.32 | 4.62 | 5.93 | 10.61 | 2.93 | 4.74 | 5.38 | 5.92 |
| 842927 | 4.24 | 0.72 | 2.96 | 5.54 | 10.03 | 6.26 | 4.59 | 5.88 |
| 824126 | 0.59 | 0.10 | 8.58 | 7.60 | 6.12 | 6.06 | 1.00 | 5.87 |
| 814662 | 1.12 | 0.19 | 5.53 | 5.17 | 7.24 | 9.88 | 1.37 | 5.84 |
| 1056172 | 10.33 | 1.77 | 6.48 | 4.11 | 1.30 | 15.30 | 1.99 | 5.84 |
| 840944 | 79.86 | 13.69 | 11.87 | 1.67 | 12.92 | 1.88 | 0.84 | 5.83 |
| 811582 | 10.13 | 1.75 | 8.01 | 4.53 | 6.69 | 4.59 | 5.11 | 5.78 |
| 1637282 | 4.01 | 0.70 | 2.86 | 2.47 | 10.87 | 9.32 | 3.03 | 5.71 |
| 838807 | 1.90 | 0.33 | 6.10 | 5.85 | 4.20 | 7.62 | 4.77 | 5.71 |
| 77915 | 14.99 | 2.63 | 3.34 | 8.82 | 3.66 | 2.66 | 10.02 | 5.70 |
| 252314 | 0.69 | 0.12 | 8.50 | 5.50 | 6.04 | 3.55 | 4.72 | 5.66 |
| 814235 | 12.37 | 2.19 | 6.17 | 6.36 | 5.75 | 7.43 | 2.52 | 5.65 |
| 461476 | 0.78 | 0.14 | 7.32 | 7.96 | 5.15 | 5.06 | 2.71 | 5.64 |
| 754358 | 12.77 | 2.26 | 4.29 | 5.26 | 8.83 | 2.50 | 7.34 | 5.64 |
| 53022 | 1.34 | 0.24 | 3.38 | 6.17 | 9.26 | 3.91 | 5.41 | 5.63 |
| 859228 | 85.06 | 15.20 | 5.32 | 4.79 | 6.57 | 7.30 | 4.01 | 5.60 |
| 290370 | 0.62 | 0.11 | 4.96 | 3.44 | 7.59 | 8.28 | 3.41 | 5.54 |
| 81331 | 2.84 | 0.52 | 1.67 | 4.49 | 1.34 | 18.34 | 1.80 | 5.53 |
| 260116 | 0.88 | 0.16 | 6.00 | 5.67 | 4.91 | 5.69 | 5.33 | 5.52 |
| 416374 | 4.49 | 0.81 | 3.17 | 8.84 | 3.61 | 10.29 | 1.67 | 5.52 |
| 840606 | 15.85 | 2.88 | 3.74 | 4.47 | 5.11 | 9.38 | 4.79 | 5.50 |
| 51773 | 11.48 | 2.09 | 6.89 | 4.08 | 3.70 | 8.77 | 4.03 | 5.50 |
| 240694 | 4.53 | 0.83 | 5.08 | 8.53 | 3.38 | 3.73 | 6.64 | 5.47 |
| 1605407 | 9.00 | 1.65 | 4.85 | 3.69 | 10.03 | 6.25 | 2.42 | 5.45 |
| 139771 | 7.72 | 1.42 | 13.10 | 8.46 | 1.72 | 1.70 | 2.24 | 5.44 |
| 1630990 | 20.64 | 3.80 | 8.87 | 4.51 | 3.52 | 9.31 | 0.98 | 5.44 |
| 1390584 | 6.54 | 1.21 | 3.13 | 4.93 | 5.15 | 12.83 | 0.99 | 5.41 |
| 186918 | 3.04 | 0.56 | 2.90 | 5.49 | 4.54 | 11.24 | 2.73 | 5.38 |
| 462097 | 8.32 | 1.55 | 7.30 | 2.88 | 1.25 | 9.95 | 5.50 | 5.38 |
| 32381 | 2.18 | 0.41 | 1.75 | 2.88 | 9.49 | 3.19 | 9.57 | 5.38 |
| 491764 | 0.81 | 0.15 | 6.00 | 5.07 | 5.46 | 5.33 | 4.98 | 5.37 |
| 1456160 | 41.61 | 7.76 | 2.23 | 3.67 | 7.39 | 11.23 | 2.29 | 5.36 |
| 159608 | 14.43 | 2.70 | 10.47 | 1.64 | 1.63 | 7.83 | 5.21 | 5.36 |
| 80050 | 8.15 | 1.52 | 6.41 | 4.16 | 11.93 | 0.95 | 3.31 | 5.35 |
| 610124 | 1.70 | 0.32 | 4.17 | 6.14 | 5.42 | 5.63 | 5.22 | 5.32 |
| 280763 | 0.53 | 0.10 | 3.60 | 5.23 | 5.40 | 6.52 | 5.64 | 5.28 |
| 283312 | 63.94 | 12.14 | 6.26 | 3.00 | 2.86 | 6.54 | 7.69 | 5.27 |
| 359781 | 7.82 | 1.49 | 3.10 | 9.11 | 3.52 | 6.46 | 4.12 | 5.26 |
| 297439 | 48.60 | 9.26 | 5.11 | 9.34 | 4.69 | 2.38 | 4.73 | 5.25 |
| 731410 | 0.53 | 0.10 | 5.74 | 4.50 | 5.23 | 5.26 | 5.48 | 5.24 |
| 772416 | 1.03 | 0.20 | 4.21 | 4.42 | 4.71 | 7.57 | 5.02 | 5.19 |
| 267419 | 0.97 | 0.19 | 3.98 | 9.05 | 3.92 | 5.50 | 3.38 | 5.16 |
| 897807 | 5.08 | 0.99 | 5.07 | 5.96 | 9.08 | 3.03 | 2.65 | 5.16 |
| 593658 | 1.08 | 0.22 | 4.01 | 5.03 | 5.25 | 5.65 | 5.29 | 5.04 |
| 511952 | 225.66 | 45.17 | 0.81 | 4.98 | 1.25 | 15.74 | 2.20 | 5.00 |
| 743452 | 7.90 | 1.59 | 7.90 | 7.46 | 2.03 | 4.53 | 2.98 | 4.98 |
| 878281 | 6.80 | 1.37 | 5.03 | 1.94 | 7.14 | 8.50 | 2.27 | 4.98 |
| 825297 | 0.96 | 0.19 | 4.48 | 3.92 | 7.31 | 4.87 | 4.28 | 4.97 |
| 731016 | 0.85 | 0.17 | 6.76 | 6.19 | 3.31 | 4.49 | 4.06 | 4.96 |
| 824723 | 14.11 | 2.84 | 3.64 | 3.92 | 5.23 | 8.15 | 3.87 | 4.96 |
| 824122 | 4.64 | 0.94 | 6.80 | 5.17 | 5.77 | 4.87 | 2.17 | 4.95 |
| 755239 | 17.93 | 3.62 | 5.25 | 3.08 | 3.43 | 8.49 | 4.50 | 4.95 |
| 784130 | 17.23 | 3.50 | 3.20 | 5.09 | 7.85 | 4.99 | 3.50 | 4.93 |
| 1585549 | 6.44 | 1.31 | 5.67 | 6.28 | 3.85 | 4.93 | 3.89 | 4.92 |
| 701677 | 16.03 | 3.27 | 7.12 | 3.55 | 3.14 | 8.18 | 2.54 | 4.91 |
| 814017 | 86.17 | 17.57 | 6.83 | 7.64 | 4.06 | 2.12 | 3.86 | 4.90 |
| 530185 | 8.46 | 1.74 | 2.68 | 5.31 | 6.79 | 3.57 | 6.04 | 4.88 |
| 773287 | 15.94 | 3.27 | 4.28 | 4.12 | 3.43 | 7.01 | 5.50 | 4.87 |
| 194723 | 5.22 | 1.07 | 8.66 | 7.32 | 2.79 | 2.88 | 2.66 | 4.86 |
| 788655 | 7.66 | 1.58 | 4.19 | 3.33 | 7.19 | 6.38 | 3.22 | 4.86 |
| 238661 | 2.74 | 0.56 | 9.41 | 7.84 | 2.13 | 2.69 | 2.21 | 4.86 |
| 773248 | 21.90 | 4.51 | 1.37 | 2.18 | 6.84 | 6.57 | 7.31 | 4.86 |
| 124252 | 6.06 | 1.25 | 11.29 | 6.83 | 1.86 | 2.10 | 2.21 | 4.85 |
| 814306 | 18.28 | 3.77 | 3.29 | 5.66 | 3.89 | 7.34 | 4.08 | 4.85 |
| 754280 | 23.83 | 4.92 | 9.79 | 5.07 | 5.33 | 1.97 | 2.08 | 4.85 |
| 1636108 | 3.58 | 0.74 | 5.59 | 4.80 | 5.19 | 4.56 | 4.09 | 4.85 |
| 461592 | 11.87 | 2.45 | 8.03 | 6.34 | 4.05 | 3.94 | 1.87 | 4.84 |
| 771053 | 16.14 | 3.34 | 3.85 | 4.29 | 4.79 | 3.56 | 7.67 | 4.83 |
| 703943 | 4.77 | 0.99 | 3.01 | 6.34 | 6.79 | 4.06 | 3.91 | 4.82 |
| 823691 | 7.42 | 1.54 | 2.71 | 4.70 | 4.41 | 8.34 | 3.93 | 4.82 |
| 289071 | 37.48 | 7.78 | 3.68 | 4.93 | 11.18 | 2.30 | 2.00 | 4.82 |
| 510576 | 132.22 | 27.52 | 5.44 | 2.70 | 2.95 | 8.26 | 4.68 | 4.81 |
| 264465 | 0.77 | 0.16 | 5.25 | 4.74 | 5.40 | 3.85 | 4.73 | 4.79 |
| 511428 | 24.31 | 5.08 | 5.64 | 3.21 | 4.36 | 5.17 | 5.56 | 4.79 |
| 488431 | 10.48 | 2.19 | 6.83 | 4.34 | 3.39 | 2.92 | 6.45 | 4.78 |
| 359701 | 5.21 | 1.09 | 4.50 | 5.09 | 3.54 | 3.24 | 7.52 | 4.78 |
| 1606837 | 23.76 | 4.98 | 6.95 | 2.67 | 3.34 | 5.80 | 5.10 | 4.77 |
| 823615 | 9.40 | 1.97 | 6.36 | 2.61 | 10.35 | 2.83 | 1.67 | 4.76 |
| 795265 | 4.58 | 0.97 | 1.65 | 3.44 | 3.71 | 12.61 | 2.30 | 4.74 |
| 813275 | 1.75 | 0.37 | 2.37 | 3.77 | 3.58 | 8.89 | 5.04 | 4.73 |
| 796351 | 2.34 | 0.50 | 5.27 | 6.47 | 3.71 | 4.50 | 3.66 | 4.72 |
| 787938 | 5.09 | 1.08 | 1.71 | 5.35 | 8.20 | 6.29 | 2.02 | 4.71 |
| 375682 | 31.04 | 6.60 | 3.43 | 4.82 | 7.07 | 3.67 | 4.53 | 4.70 |
| 869450 | 51.58 | 10.98 | 10.12 | 2.49 | 0.92 | 4.42 | 5.54 | 4.70 |
| 251435 | 0.73 | 0.16 | 4.68 | 5.34 | 5.04 | 4.18 | 4.15 | 4.68 |
| 824267 | 0.74 | 0.16 | 4.49 | 4.65 | 4.35 | 4.86 | 5.03 | 4.68 |
| 214006 | 4.67 | 1.00 | 9.34 | 7.13 | 2.17 | 3.26 | 1.38 | 4.66 |
| 251517 | 0.73 | 0.16 | 4.87 | 4.91 | 5.29 | 3.94 | 4.18 | 4.64 |
| 609052 | 0.87 | 0.19 | 5.08 | 4.85 | 3.84 | 4.06 | 5.34 | 4.63 |
| 1466631 | 1.21 | 0.26 | 3.47 | 5.67 | 4.78 | 4.73 | 4.50 | 4.63 |
| 1500000 | 6.11 | 1.32 | 8.25 | 6.21 | 3.19 | 2.69 | 2.81 | 4.63 |
| 1055201 | 30.25 | 6.55 | 4.44 | 2.37 | 4.47 | 9.42 | 2.38 | 4.62 |
| 840878 | 25.81 | 5.59 | 2.71 | 4.00 | 3.35 | 9.63 | 3.38 | 4.62 |
| 773204 | 21.34 | 4.63 | 2.70 | 3.84 | 5.75 | 1.24 | 9.53 | 4.61 |
| 290841 | 6.35 | 1.38 | 7.54 | 6.98 | 2.99 | 3.39 | 2.13 | 4.61 |
| 134969 | 6.81 | 1.49 | 10.24 | 6.19 | 2.21 | 2.17 | 2.11 | 4.58 |
| 824376 | 0.49 | 0.11 | 5.18 | 4.11 | 6.01 | 6.67 | 0.94 | 4.58 |
| 768643 | 41.57 | 9.10 | 2.42 | 4.02 | 11.03 | 2.91 | 2.45 | 4.57 |
| 26617 | 4.62 | 1.01 | 1.74 | 7.29 | 3.24 | 7.49 | 3.02 | 4.56 |
| 305538 | 11.44 | 2.54 | 6.22 | 5.46 | 2.58 | 2.41 | 5.84 | 4.50 |
| 811162 | 6.36 | 1.41 | 4.62 | 2.07 | 5.28 | 2.80 | 7.76 | 4.50 |
| 1637343 | 5.03 | 1.12 | 3.26 | 4.54 | 4.14 | 6.66 | 3.91 | 4.50 |
| 197779 | 8.61 | 1.91 | 10.37 | 7.03 | 1.68 | 1.51 | 1.91 | 4.50 |
| 815549 | 2.30 | 0.51 | 6.84 | 5.83 | 3.95 | 4.46 | 1.38 | 4.49 |
| 51700 | 3.66 | 0.82 | 4.47 | 4.07 | 4.99 | 3.08 | 5.73 | 4.47 |
| 757206 | 5.25 | 1.18 | 3.96 | 4.49 | 6.00 | 3.91 | 3.98 | 4.47 |
| 838761 | 2.59 | 0.58 | 6.93 | 6.35 | 2.56 | 3.70 | 2.80 | 4.47 |
| 343987 | 11.55 | 2.59 | 2.47 | 4.92 | 6.23 | 2.51 | 6.15 | 4.46 |
| 767406 | 0.44 | 0.10 | 5.11 | 3.93 | 4.22 | 4.46 | 4.52 | 4.45 |
| 79032 | 37.58 | 8.53 | 2.88 | 8.31 | 5.01 | 3.70 | 2.12 | 4.41 |
| 815036 | 1.62 | 0.37 | 5.65 | 5.86 | 3.71 | 4.71 | 2.08 | 4.40 |
| 823895 | 1.57 | 0.36 | 4.08 | 4.25 | 4.38 | 4.82 | 4.45 | 4.40 |
| 201282 | 6.42 | 1.46 | 1.85 | 6.04 | 4.99 | 6.43 | 2.63 | 4.39 |
| 773208 | 26.40 | 6.05 | 2.45 | 3.37 | 5.47 | 1.22 | 9.32 | 4.37 |
| 121436 | 6.37 | 1.46 | 7.91 | 6.01 | 2.52 | 3.24 | 2.09 | 4.36 |
| 238821 | 6.54 | 1.50 | 5.59 | 1.57 | 4.76 | 0.92 | 8.90 | 4.35 |
| 814798 | 32.86 | 7.58 | 3.15 | 4.62 | 2.68 | 9.26 | 1.99 | 4.34 |
| 196650 | 10.93 | 2.52 | 4.17 | 6.60 | 2.20 | 6.71 | 1.99 | 4.33 |
| 971367 | 35.22 | 8.15 | 4.78 | 2.95 | 2.51 | 8.35 | 3.04 | 4.32 |
| 841220 | 1.46 | 0.34 | 3.52 | 5.00 | 4.19 | 4.54 | 4.37 | 4.32 |
| 200599 | 13.42 | 3.11 | 5.88 | 2.44 | 8.89 | 1.51 | 2.87 | 4.32 |
| 487733 | 15.01 | 3.48 | 3.75 | 5.14 | 4.04 | 6.05 | 2.61 | 4.32 |
| 564537 | 1.79 | 0.42 | 2.77 | 5.08 | 5.29 | 4.04 | 4.38 | 4.31 |
| 50606 | 14.94 | 3.47 | 5.20 | 2.99 | 5.04 | 3.92 | 4.39 | 4.31 |
| 253314 | 1.52 | 0.35 | 7.93 | 5.83 | 2.29 | 2.55 | 2.92 | 4.30 |
| 417908 | 12.10 | 2.82 | 8.40 | 4.93 | 3.98 | 1.81 | 2.34 | 4.29 |
| 29063 | 3.34 | 0.78 | 2.94 | 6.06 | 4.11 | 3.85 | 4.48 | 4.29 |
| 773345 | 1.21 | 0.28 | 3.42 | 4.56 | 2.89 | 8.45 | 2.12 | 4.29 |
| 1592006 | 5.30 | 1.24 | 4.98 | 3.66 | 4.06 | 4.48 | 4.22 | 4.28 |
| 461499 | 2.26 | 0.53 | 2.94 | 3.53 | 6.30 | 7.11 | 1.48 | 4.27 |
| 815800 | 1.22 | 0.29 | 5.36 | 6.04 | 5.43 | 3.31 | 1.13 | 4.25 |
| 436769 | 168.72 | 39.68 | 3.44 | 3.03 | 2.92 | 5.33 | 6.54 | 4.25 |
| 427838 | 4.05 | 0.95 | 7.99 | 6.36 | 2.29 | 2.32 | 2.29 | 4.25 |
| 50188 | 10.75 | 2.53 | 4.92 | 4.45 | 4.01 | 3.41 | 4.46 | 4.25 |
| 1519515 | 0.85 | 0.20 | 3.89 | 3.77 | 5.31 | 3.37 | 4.90 | 4.25 |
| 138444 | 4.04 | 0.95 | 3.84 | 7.52 | 3.07 | 4.21 | 2.54 | 4.24 |
| 1631849 | 25.01 | 5.91 | 4.16 | 5.55 | 3.42 | 4.66 | 3.40 | 4.24 |
| 613237 | 1.07 | 0.25 | 3.02 | 4.22 | 3.83 | 5.08 | 4.94 | 4.22 |
| 358850 | 21.29 | 5.06 | 7.24 | 3.98 | 3.18 | 2.13 | 4.52 | 4.21 |
| 290378 | 17.10 | 4.07 | 3.83 | 3.50 | 3.92 | 3.90 | 5.86 | 4.20 |
| 898109 | 6.77 | 1.62 | 4.54 | 3.37 | 5.89 | 5.18 | 1.97 | 4.19 |
| 82734 | 7.21 | 1.73 | 3.01 | 4.42 | 4.26 | 6.62 | 2.60 | 4.18 |
| 450307 | 9.39 | 2.25 | 7.70 | 6.02 | 3.75 | 1.27 | 2.10 | 4.17 |
| 593929 | 17.74 | 4.26 | 6.37 | 2.52 | 9.06 | 1.63 | 1.24 | 4.17 |
| 587415 | 12.98 | 3.12 | 7.46 | 3.53 | 7.08 | 1.19 | 1.53 | 4.16 |
| 842882 | 2.20 | 0.53 | 3.81 | 5.06 | 5.56 | 2.68 | 3.67 | 4.16 |
| 896914 | 18.03 | 4.35 | 5.29 | 7.59 | 0.53 | 6.00 | 1.32 | 4.14 |
| 1607765 | 3.96 | 0.96 | 4.88 | 3.54 | 3.13 | 5.17 | 3.98 | 4.14 |
| 824329 | 1.94 | 0.47 | 4.04 | 4.22 | 5.97 | 3.83 | 2.60 | 4.13 |
| 1558411 | 8.20 | 1.99 | 0.59 | 1.24 | 9.68 | 5.69 | 3.37 | 4.11 |
| 453599 | 4.23 | 1.03 | 3.89 | 3.56 | 3.50 | 6.56 | 3.01 | 4.11 |
| 486538 | 7.64 | 1.86 | 3.08 | 5.14 | 3.85 | 5.78 | 2.65 | 4.10 |
| 712216 | 0.50 | 0.12 | 5.35 | 4.81 | 4.78 | 4.53 | 1.03 | 4.10 |
| 753162 | 2.81 | 0.69 | 2.94 | 3.35 | 2.20 | 9.53 | 2.46 | 4.10 |
| 1637296 | 25.52 | 6.23 | 4.17 | 2.86 | 2.82 | 6.06 | 4.57 | 4.09 |
| 898242 | 10.34 | 2.53 | 2.92 | 4.71 | 4.80 | 5.15 | 2.87 | 4.09 |
| 771048 | 10.54 | 2.58 | 5.50 | 3.59 | 3.73 | 3.20 | 4.39 | 4.08 |
| 951125 | 18.86 | 4.63 | 3.30 | 7.80 | 2.31 | 3.51 | 3.45 | 4.07 |
| 611150 | 7.99 | 1.96 | 4.60 | 2.92 | 4.15 | 5.04 | 3.66 | 4.07 |
| 842925 | 4.61 | 1.13 | 3.78 | 3.80 | 4.94 | 4.96 | 2.87 | 4.07 |
| 824843 | 4.03 | 0.99 | 5.28 | 3.89 | 3.11 | 6.61 | 1.41 | 4.06 |
| 878544 | 19.20 | 4.73 | 8.54 | 2.29 | 4.85 | 2.59 | 2.02 | 4.06 |
| 781366 | 12.06 | 2.98 | 4.50 | 3.42 | 1.61 | 2.86 | 7.87 | 4.05 |
| 79000 | 6.24 | 1.54 | 2.16 | 5.95 | 3.81 | 5.31 | 3.04 | 4.05 |
| 24208 | 7.49 | 1.85 | 3.19 | 3.67 | 3.57 | 4.54 | 5.28 | 4.05 |
| 729953 | 4.95 | 1.23 | 3.26 | 4.43 | 3.91 | 6.32 | 2.25 | 4.04 |
| 34134 | 2.73 | 0.68 | 6.48 | 3.68 | 4.71 | 3.19 | 2.08 | 4.03 |
| 745283 | 7.94 | 1.97 | 4.03 | 3.25 | 3.72 | 4.68 | 4.44 | 4.02 |
| 1602619 | 135.32 | 33.71 | 4.29 | 3.43 | 3.41 | 4.71 | 4.23 | 4.02 |
| 1636868 | 5.07 | 1.27 | 5.31 | 4.50 | 3.81 | 4.48 | 1.96 | 4.01 |
| 726483 | 3.50 | 0.87 | 2.71 | 4.78 | 2.55 | 5.48 | 4.52 | 4.01 |
| 360403 | 28.16 | 7.03 | 9.60 | 1.99 | 5.91 | 1.37 | 1.17 | 4.01 |
| 757462 | 12.77 | 3.19 | 4.05 | 4.75 | 4.11 | 4.73 | 2.41 | 4.01 |
| 279482 | 8.66 | 2.16 | 3.13 | 2.00 | 10.92 | 1.42 | 2.54 | 4.00 |
| 811766 | 4.93 | 1.23 | 1.80 | 3.53 | 4.42 | 6.47 | 3.79 | 4.00 |
| 50764 | 0.43 | 0.11 | 4.10 | 3.54 | 3.73 | 5.17 | 3.48 | 4.00 |
| 626716 | 21.60 | 5.41 | 2.78 | 6.84 | 3.77 | 3.86 | 2.73 | 4.00 |
| 213682 | 13.07 | 3.27 | 0.79 | 3.49 | 2.68 | 9.05 | 3.96 | 3.99 |
| 685922 | 3.59 | 0.90 | 3.85 | 3.96 | 3.20 | 5.02 | 3.93 | 3.99 |
| 1631194 | 21.83 | 5.47 | 5.50 | 1.73 | 5.19 | 6.07 | 1.46 | 3.99 |
| 823578 | 0.56 | 0.14 | 4.98 | 3.43 | 4.18 | 3.36 | 3.93 | 3.98 |
| 32509 | 4.38 | 1.10 | 1.50 | 7.60 | 1.95 | 4.07 | 4.72 | 3.97 |
| 845352 | 13.36 | 3.37 | 4.44 | 3.86 | 3.95 | 2.54 | 5.05 | 3.97 |
| 191978 | 47.95 | 12.11 | 3.93 | 4.27 | 3.76 | 5.36 | 2.48 | 3.96 |
| 120707 | 19.60 | 4.95 | 2.32 | 3.87 | 3.65 | 8.32 | 1.62 | 3.96 |
| 75415 | 51.71 | 13.09 | 4.45 | 4.47 | 2.60 | 4.53 | 3.70 | 3.95 |
| 186768 | 9.43 | 2.39 | 2.81 | 1.73 | 11.34 | 2.18 | 1.69 | 3.95 |
| 80484 | 14.69 | 3.72 | 9.35 | 2.80 | 1.75 | 3.52 | 2.32 | 3.95 |
| 1475200 | 28.16 | 7.14 | 1.70 | 3.21 | 6.52 | 2.96 | 5.34 | 3.95 |
| 810510 | 27.71 | 7.03 | 3.76 | 3.64 | 2.68 | 4.64 | 5.00 | 3.94 |
| 897690 | 15.87 | 4.03 | 5.68 | 4.08 | 4.14 | 1.92 | 3.87 | 3.94 |
| 45327 | 8.18 | 2.08 | 2.60 | 3.75 | 4.04 | 5.11 | 4.18 | 3.94 |
| 299723 | 2.98 | 0.76 | 1.71 | 2.92 | 5.37 | 4.69 | 4.93 | 3.93 |
| 81578 | 12.77 | 3.25 | 5.02 | 3.62 | 6.26 | 2.36 | 2.37 | 3.93 |
| 197888 | 4.97 | 1.27 | 4.11 | 2.76 | 2.65 | 3.61 | 6.49 | 3.92 |
| 810571 | 4.37 | 1.11 | 2.29 | 5.21 | 2.51 | 6.03 | 3.57 | 3.92 |
| 768443 | 26.58 | 6.79 | 2.54 | 4.37 | 3.56 | 7.05 | 2.07 | 3.92 |
| 150314 | 5.29 | 1.35 | 2.49 | 4.79 | 3.04 | 6.51 | 2.76 | 3.92 |
| 196189 | 11.19 | 2.86 | 4.68 | 4.67 | 3.98 | 2.71 | 3.52 | 3.91 |
| 626990 | 7.39 | 1.89 | 2.81 | 2.77 | 3.38 | 7.85 | 2.73 | 3.91 |
| 127396 | 14.07 | 3.61 | 3.73 | 5.03 | 1.41 | 6.75 | 2.59 | 3.90 |
| 49710 | 3.21 | 0.82 | 2.93 | 3.48 | 4.65 | 4.08 | 4.34 | 3.90 |
| 262864 | 36.97 | 9.52 | 3.65 | 2.59 | 3.11 | 5.73 | 4.36 | 3.89 |
| 815737 | 3.64 | 0.94 | 3.91 | 4.25 | 3.43 | 5.08 | 2.74 | 3.88 |
| 256680 | 1.72 | 0.44 | 3.22 | 3.59 | 3.08 | 4.91 | 4.60 | 3.88 |
| 461509 | 0.62 | 0.16 | 4.43 | 4.30 | 3.23 | 5.77 | 1.64 | 3.87 |
| 809578 | 24.75 | 6.40 | 5.73 | 2.71 | 3.10 | 4.48 | 3.32 | 3.87 |
| 811121 | 1.07 | 0.28 | 2.85 | 4.21 | 4.20 | 4.46 | 3.62 | 3.87 |
| 547058 | 9.57 | 2.48 | 4.03 | 2.83 | 3.49 | 4.83 | 4.11 | 3.86 |
| 1035765 | 6.21 | 1.61 | 3.91 | 1.25 | 4.74 | 0.71 | 8.68 | 3.86 |
| 824739 | 2.63 | 0.68 | 7.31 | 4.22 | 2.34 | 3.52 | 1.89 | 3.86 |
| 815017 | 14.81 | 3.84 | 3.95 | 3.11 | 3.65 | 4.61 | 3.95 | 3.85 |
| 32684 | 24.32 | 6.32 | 4.79 | 1.68 | 2.94 | 5.24 | 4.60 | 3.85 |
| 178137 | 238.41 | 61.93 | 3.66 | 2.98 | 2.56 | 6.38 | 3.66 | 3.85 |
| 282283 | 0.84 | 0.22 | 3.28 | 3.36 | 3.61 | 4.89 | 4.09 | 3.85 |
| 725978 | 4.92 | 1.28 | 5.17 | 3.07 | 2.63 | 4.05 | 4.31 | 3.85 |
| 1631762 | 46.32 | 12.06 | 2.78 | 4.97 | 2.53 | 4.94 | 4.00 | 3.84 |
| 1553306 | 7.92 | 2.06 | 3.60 | 3.69 | 3.79 | 4.48 | 3.64 | 3.84 |
| 590145 | 4.37 | 1.14 | 3.78 | 3.14 | 3.77 | 5.80 | 2.69 | 3.84 |
| 815230 | 9.59 | 2.50 | 3.37 | 6.27 | 3.72 | 3.68 | 2.14 | 3.84 |
| 824237 | 74.89 | 19.54 | 3.14 | 3.18 | 3.55 | 5.72 | 3.57 | 3.83 |
| 361048 | 3.13 | 0.82 | 2.53 | 3.04 | 6.30 | 4.44 | 2.84 | 3.83 |
| 417867 | 13.52 | 3.54 | 2.81 | 3.70 | 3.15 | 4.96 | 4.47 | 3.82 |
| 878152 | 4.20 | 1.10 | 3.49 | 3.90 | 4.76 | 3.69 | 3.25 | 3.82 |
| 487499 | 4.41 | 1.16 | 2.68 | 3.09 | 8.83 | 3.10 | 1.37 | 3.82 |
| 712570 | 1.32 | 0.35 | 4.30 | 4.75 | 4.52 | 3.37 | 2.10 | 3.81 |
| 124824 | 30.85 | 8.13 | 3.79 | 2.43 | 2.17 | 3.37 | 7.23 | 3.80 |
| 626385 | 15.94 | 4.20 | 2.97 | 4.37 | 4.40 | 2.13 | 5.11 | 3.79 |
| 813271 | 0.96 | 0.25 | 3.16 | 4.24 | 3.70 | 4.31 | 3.56 | 3.79 |
| 561971 | 3.81 | 1.01 | 2.50 | 4.39 | 4.74 | 3.83 | 3.46 | 3.79 |
| 824358 | 0.96 | 0.25 | 4.80 | 3.84 | 3.71 | 5.67 | 0.90 | 3.79 |
| 741067 | 64.30 | 17.00 | 0.88 | 4.25 | 2.30 | 7.09 | 4.38 | 3.78 |
| 46411 | 3.07 | 0.81 | 2.37 | 2.25 | 5.61 | 3.01 | 5.66 | 3.78 |
| 594517 | 24.81 | 6.57 | 4.24 | 1.83 | 7.82 | 1.45 | 3.53 | 3.78 |
| 825668 | 2.26 | 0.60 | 3.80 | 3.91 | 4.97 | 3.99 | 2.20 | 3.77 |
| 362926 | 4.39 | 1.17 | 1.58 | 4.23 | 3.24 | 2.08 | 7.71 | 3.77 |
| 814018 | 0.98 | 0.26 | 3.30 | 3.43 | 6.07 | 5.33 | 0.71 | 3.77 |
| 745503 | 42.62 | 11.32 | 2.92 | 3.95 | 3.33 | 3.03 | 5.59 | 3.77 |
| 884531 | 7.71 | 2.05 | 2.78 | 3.56 | 3.60 | 6.93 | 1.94 | 3.76 |
| 210862 | 2.56 | 0.68 | 5.33 | 3.36 | 3.68 | 3.66 | 2.76 | 3.76 |
| 504544 | 12.31 | 3.28 | 2.35 | 4.09 | 2.54 | 7.86 | 1.95 | 3.76 |
| 838668 | 3.58 | 0.95 | 1.88 | 5.28 | 2.47 | 7.20 | 1.95 | 3.76 |
| 433253 | 7.29 | 1.95 | 2.88 | 2.93 | 2.56 | 6.57 | 3.78 | 3.74 |
| 461336 | 3.42 | 0.91 | 3.19 | 3.49 | 3.78 | 5.20 | 3.05 | 3.74 |
| 878525 | 11.10 | 2.97 | 4.03 | 4.61 | 3.75 | 2.79 | 3.49 | 3.74 |
| 383185 | 12.04 | 3.22 | 6.71 | 2.73 | 7.18 | 1.05 | 1.01 | 3.73 |
| 897733 | 5.86 | 1.58 | 4.19 | 4.06 | 4.13 | 4.55 | 1.60 | 3.71 |
| 1594019 | 24.27 | 6.55 | 3.80 | 3.42 | 3.34 | 3.87 | 4.10 | 3.71 |
| 506564 | 12.93 | 3.49 | 5.12 | 3.30 | 3.70 | 2.28 | 4.10 | 3.70 |
| 824917 | 1.74 | 0.47 | 4.19 | 3.64 | 3.11 | 2.99 | 4.52 | 3.69 |
| 84955 | 5.26 | 1.43 | 5.70 | 4.55 | 4.49 | 1.79 | 1.92 | 3.69 |
| 951080 | 2.43 | 0.66 | 6.73 | 4.60 | 2.27 | 2.30 | 2.53 | 3.69 |
| 30428 | 4.29 | 1.16 | 0.76 | 1.72 | 2.50 | 10.04 | 3.40 | 3.68 |
| 784093 | 6.44 | 1.75 | 3.21 | 4.16 | 4.32 | 2.79 | 3.93 | 3.68 |
| 135900 | 9.44 | 2.57 | 9.57 | 1.87 | 4.30 | 1.31 | 1.34 | 3.68 |
| 950688 | 52.65 | 14.32 | 4.34 | 2.16 | 2.56 | 4.14 | 5.19 | 3.68 |
| 878284 | 4.41 | 1.20 | 5.36 | 2.63 | 2.49 | 1.62 | 6.22 | 3.66 |
| 845363 | 29.59 | 8.08 | 4.04 | 3.09 | 2.09 | 5.22 | 3.87 | 3.66 |
| 358456 | 37.98 | 10.38 | 4.06 | 4.52 | 2.15 | 4.94 | 2.64 | 3.66 |
| 76252 | 11.64 | 3.19 | 5.18 | 3.55 | 3.64 | 3.20 | 2.69 | 3.65 |
| 36607 | 7.52 | 2.06 | 4.19 | 3.70 | 2.34 | 5.99 | 2.02 | 3.65 |
| 624271 | 5.74 | 1.58 | 3.97 | 2.76 | 2.08 | 7.77 | 1.62 | 3.64 |
| 743071 | 1.89 | 0.52 | 4.55 | 1.78 | 1.17 | 8.79 | 1.87 | 3.63 |
| 1070015 | 3.26 | 0.90 | 1.10 | 3.25 | 0.84 | 5.24 | 7.71 | 3.63 |
| 203132 | 12.63 | 3.48 | 2.95 | 2.71 | 2.51 | 4.70 | 5.27 | 3.63 |
| 259953 | 1.46 | 0.40 | 3.59 | 3.86 | 2.56 | 4.66 | 3.46 | 3.63 |
| 814427 | 0.47 | 0.13 | 3.78 | 3.80 | 3.96 | 5.65 | 0.91 | 3.62 |
| 259462 | 3.69 | 1.02 | 1.07 | 1.43 | 1.22 | 13.31 | 1.06 | 3.62 |
| 810558 | 14.49 | 4.01 | 2.38 | 3.34 | 3.00 | 3.69 | 5.66 | 3.61 |
| 51151 | 3.26 | 0.90 | 3.04 | 4.60 | 2.81 | 5.13 | 2.49 | 3.61 |
| 272750 | 20.57 | 5.70 | 3.98 | 5.64 | 2.94 | 2.81 | 2.70 | 3.61 |
| 360885 | 40.29 | 11.17 | 5.08 | 2.71 | 5.71 | 1.52 | 3.03 | 3.61 |
| 208082 | 7.90 | 2.19 | 2.97 | 6.19 | 3.18 | 2.98 | 2.71 | 3.61 |
| 627428 | 24.22 | 6.72 | 1.13 | 1.36 | 6.42 | 4.29 | 4.83 | 3.61 |
| 845345 | 5.09 | 1.41 | 2.54 | 3.32 | 3.32 | 4.11 | 4.73 | 3.61 |
| 417801 | 17.18 | 4.77 | 2.94 | 2.40 | 2.51 | 5.34 | 4.83 | 3.60 |
| 361922 | 9.18 | 2.55 | 3.94 | 3.22 | 3.20 | 3.45 | 4.16 | 3.60 |
| 898206 | 1.79 | 0.50 | 2.31 | 3.99 | 3.53 | 4.38 | 3.77 | 3.60 |
| 431296 | 1.85 | 0.51 | 2.75 | 3.55 | 5.68 | 1.98 | 4.01 | 3.59 |
| 587595 | 559.22 | 155.61 | 0.86 | 2.83 | 3.47 | 7.37 | 3.44 | 3.59 |
| 1632161 | 39.75 | 11.08 | 4.86 | 3.58 | 2.47 | 2.92 | 4.10 | 3.59 |
| 121196 | 22.80 | 6.36 | 2.64 | 3.37 | 3.11 | 3.53 | 5.26 | 3.58 |
| 823819 | 11.96 | 3.34 | 4.72 | 4.29 | 2.21 | 3.08 | 3.60 | 3.58 |
| 1456721 | 12.82 | 3.58 | 3.28 | 4.85 | 3.29 | 3.04 | 3.47 | 3.58 |
| 1500324 | 6.77 | 1.89 | 2.74 | 6.90 | 3.33 | 2.21 | 2.73 | 3.58 |
| 86160 | 2.10 | 0.59 | 3.16 | 4.11 | 3.48 | 3.18 | 3.96 | 3.58 |
| 246620 | 16.85 | 4.71 | 3.66 | 3.89 | 3.47 | 5.00 | 1.89 | 3.58 |
| 52730 | 6.16 | 1.72 | 2.03 | 3.11 | 2.60 | 6.50 | 3.64 | 3.58 |
| 809657 | 4.72 | 1.32 | 5.12 | 2.96 | 2.52 | 2.52 | 4.76 | 3.58 |
| 253577 | 1.48 | 0.41 | 4.99 | 4.45 | 2.01 | 2.88 | 3.54 | 3.57 |
| 486459 | 6.24 | 1.75 | 2.96 | 3.80 | 3.21 | 5.49 | 2.40 | 3.57 |
| 23576 | 3.58 | 1.00 | 2.58 | 4.47 | 3.35 | 4.26 | 3.17 | 3.57 |
| 119403 | 1.22 | 0.34 | 3.95 | 4.15 | 2.67 | 3.43 | 3.62 | 3.57 |
| 324342 | 4.41 | 1.24 | 4.11 | 3.07 | 4.52 | 3.34 | 2.80 | 3.57 |
| 447518 | 10.98 | 3.08 | 1.93 | 4.33 | 4.07 | 2.68 | 4.81 | 3.56 |
| 811953 | 17.84 | 5.02 | 3.87 | 4.29 | 4.39 | 2.34 | 2.86 | 3.55 |
| 824124 | 1.03 | 0.29 | 4.84 | 3.47 | 4.24 | 4.08 | 1.11 | 3.55 |
| 837953 | 10.44 | 2.95 | 2.00 | 4.04 | 3.97 | 5.08 | 2.63 | 3.55 |
| 511521 | 33.96 | 9.58 | 2.43 | 4.75 | 3.58 | 3.11 | 3.86 | 3.54 |
| 1493252 | 9.89 | 2.79 | 3.88 | 4.49 | 1.98 | 5.20 | 2.16 | 3.54 |
| 626640 | 8.33 | 2.35 | 2.29 | 3.09 | 5.39 | 3.53 | 3.42 | 3.54 |
| 22731 | 4.46 | 1.26 | 3.38 | 2.26 | 3.14 | 6.00 | 2.92 | 3.54 |
| 84022 | 6.97 | 1.97 | 5.71 | 3.40 | 2.86 | 3.42 | 2.30 | 3.54 |
| 841302 | 7.41 | 2.09 | 2.18 | 2.19 | 10.28 | 1.46 | 1.58 | 3.54 |
| 150262 | 13.66 | 3.87 | 3.31 | 4.40 | 3.92 | 2.96 | 3.05 | 3.53 |
| 950461 | 38.86 | 11.02 | 0.81 | 1.10 | 5.41 | 4.59 | 5.73 | 3.53 |
| 251019 | 45.62 | 12.95 | 2.89 | 2.88 | 6.72 | 1.14 | 4.00 | 3.52 |
| 67654 | 15.79 | 4.50 | 1.22 | 3.32 | 2.33 | 6.57 | 4.11 | 3.51 |
| 592403 | 0.46 | 0.13 | 0.83 | 2.74 | 2.70 | 8.19 | 3.04 | 3.50 |
| 755750 | 5.88 | 1.68 | 3.23 | 1.83 | 2.12 | 7.95 | 2.36 | 3.50 |
| 1420790 | 13.40 | 3.84 | 2.46 | 3.91 | 3.17 | 3.47 | 4.47 | 3.49 |
| 843352 | 28.44 | 8.17 | 2.97 | 3.18 | 2.69 | 3.78 | 4.80 | 3.48 |
| 1604342 | 1.97 | 0.57 | 1.56 | 2.15 | 1.34 | 9.00 | 3.33 | 3.48 |
| 258167 | 17.24 | 4.96 | 3.26 | 5.42 | 3.43 | 2.37 | 2.90 | 3.48 |
| 308466 | 9.26 | 2.67 | 4.61 | 4.96 | 3.31 | 1.52 | 2.95 | 3.47 |
| 272576 | 6.22 | 1.80 | 1.34 | 3.85 | 4.11 | 4.71 | 3.25 | 3.45 |
| 108265 | 7.24 | 2.10 | 4.59 | 3.00 | 3.05 | 3.84 | 2.76 | 3.45 |
| 842784 | 326.54 | 94.88 | 0.85 | 3.11 | 1.95 | 7.01 | 4.30 | 3.44 |
| 1467207 | 3.68 | 1.07 | 2.33 | 4.23 | 4.08 | 2.71 | 3.86 | 3.44 |
| 784225 | 10.62 | 3.09 | 2.36 | 4.44 | 3.93 | 2.57 | 3.89 | 3.44 |
| 430179 | 51.95 | 15.13 | 2.52 | 2.04 | 9.04 | 1.80 | 1.78 | 3.44 |
| 796513 | 17.83 | 5.20 | 4.20 | 3.32 | 2.84 | 5.24 | 1.56 | 3.43 |
| 83363 | 5.08 | 1.48 | 3.97 | 3.58 | 1.64 | 3.63 | 4.33 | 3.43 |
| 489109 | 7.44 | 2.17 | 2.33 | 3.11 | 5.81 | 2.19 | 3.71 | 3.43 |
| 812977 | 7.62 | 2.22 | 2.38 | 2.15 | 6.48 | 4.79 | 1.35 | 3.43 |
| 1492238 | 13.96 | 4.08 | 4.19 | 3.72 | 2.61 | 4.20 | 2.41 | 3.43 |
| 740914 | 7.20 | 2.11 | 5.02 | 1.70 | 6.97 | 1.58 | 1.84 | 3.42 |
| 1475797 | 85.21 | 24.92 | 1.93 | 2.41 | 2.93 | 7.69 | 2.14 | 3.42 |
| 897042 | 4.95 | 1.45 | 6.12 | 3.62 | 2.70 | 3.57 | 1.07 | 3.42 |
| 249705 | 62.65 | 18.34 | 2.89 | 3.73 | 2.52 | 4.92 | 3.02 | 3.42 |
| 1476065 | 6.33 | 1.86 | 2.32 | 4.22 | 1.08 | 8.04 | 1.42 | 3.41 |
| 251731 | 0.82 | 0.24 | 2.88 | 3.28 | 3.37 | 3.75 | 3.78 | 3.41 |
| 838366 | 11.15 | 3.27 | 2.46 | 3.30 | 3.45 | 3.28 | 4.57 | 3.41 |
| 855799 | 9.00 | 2.64 | 2.70 | 3.06 | 2.67 | 2.66 | 5.96 | 3.41 |
| 701112 | 8.70 | 2.55 | 2.26 | 4.01 | 3.63 | 4.37 | 2.78 | 3.41 |
| 26406 | 6.56 | 1.92 | 2.41 | 3.58 | 3.68 | 3.80 | 3.58 | 3.41 |
| 727251 | 34.32 | 10.07 | 2.95 | 3.27 | 4.52 | 4.01 | 2.30 | 3.41 |
| 252453 | 20.12 | 5.91 | 2.32 | 4.20 | 2.91 | 5.06 | 2.54 | 3.41 |
| 1435185 | 2.56 | 0.75 | 5.55 | 2.80 | 3.10 | 3.49 | 2.08 | 3.40 |
| 741474 | 4.93 | 1.45 | 2.81 | 2.16 | 3.51 | 5.40 | 3.13 | 3.40 |
| 289891 | 7.82 | 2.30 | 3.60 | 2.40 | 7.51 | 1.93 | 1.56 | 3.40 |
| 796774 | 0.44 | 0.13 | 3.55 | 2.86 | 3.04 | 3.54 | 4.02 | 3.40 |
| 290642 | 7.76 | 2.28 | 4.70 | 3.05 | 2.60 | 3.37 | 3.27 | 3.40 |
| 149013 | 7.82 | 2.30 | 2.27 | 1.81 | 4.99 | 1.02 | 6.89 | 3.40 |
| 745296 | 29.18 | 8.59 | 7.43 | 1.72 | 1.39 | 4.51 | 1.94 | 3.40 |
| 950603 | 3.53 | 1.04 | 1.95 | 2.17 | 2.24 | 8.88 | 1.70 | 3.39 |
| 878403 | 7.20 | 2.13 | 3.07 | 4.92 | 1.88 | 5.02 | 2.03 | 3.38 |
| 950430 | 7.48 | 2.22 | 4.11 | 4.50 | 2.71 | 2.81 | 2.76 | 3.38 |
| 39821 | 5.84 | 1.73 | 2.98 | 3.80 | 3.61 | 3.36 | 3.14 | 3.38 |
| 843139 | 39.96 | 11.84 | 4.71 | 1.39 | 4.05 | 2.96 | 3.77 | 3.38 |
| 137189 | 5.26 | 1.56 | 2.28 | 4.02 | 3.12 | 4.62 | 2.80 | 3.37 |
| 296529 | 9.83 | 2.92 | 3.71 | 4.18 | 2.72 | 2.15 | 4.08 | 3.37 |
| 544639 | 2.82 | 0.84 | 4.23 | 3.65 | 3.42 | 1.95 | 3.59 | 3.37 |
| 841140 | 2.47 | 0.73 | 4.27 | 4.30 | 2.28 | 3.23 | 2.75 | 3.36 |
| 49728 | 1.27 | 0.38 | 4.06 | 3.75 | 2.82 | 2.96 | 3.23 | 3.36 |
| 1638852 | 1.21 | 0.36 | 3.84 | 4.80 | 3.21 | 2.46 | 2.52 | 3.36 |
| 51011 | 4.64 | 1.38 | 3.25 | 1.40 | 2.40 | 2.89 | 6.87 | 3.36 |
| 753092 | 7.06 | 2.10 | 2.28 | 4.55 | 3.07 | 4.01 | 2.88 | 3.36 |
| 898210 | 8.13 | 2.42 | 2.34 | 3.18 | 3.05 | 4.51 | 3.70 | 3.36 |
| 490753 | 5.35 | 1.60 | 2.63 | 3.40 | 2.73 | 6.39 | 1.60 | 3.35 |
| 855624 | 17.75 | 5.31 | 1.02 | 1.19 | 0.83 | 1.13 | 12.53 | 3.34 |
| 768395 | 14.29 | 4.28 | 4.43 | 3.97 | 3.60 | 2.60 | 2.09 | 3.34 |
| 212165 | 41.96 | 12.58 | 5.13 | 3.22 | 4.38 | 1.81 | 2.15 | 3.34 |
| 377246 | 12.60 | 3.78 | 2.94 | 2.21 | 9.10 | 0.91 | 1.52 | 3.34 |
| 809876 | 27.74 | 8.32 | 4.70 | 3.46 | 1.68 | 2.47 | 4.37 | 3.34 |
| 462325 | 8.76 | 2.63 | 2.80 | 0.87 | 2.51 | 7.67 | 2.81 | 3.33 |
| 624785 | 6.17 | 1.86 | 3.38 | 3.81 | 2.96 | 4.60 | 1.16 | 3.32 |
| 291345 | 70.40 | 21.23 | 7.87 | 1.97 | 3.76 | 1.35 | 1.64 | 3.32 |
| 257382 | 12.80 | 3.86 | 3.91 | 3.02 | 5.42 | 2.19 | 2.05 | 3.32 |
| 700900 | 9.94 | 3.00 | 3.26 | 2.88 | 3.29 | 3.74 | 3.40 | 3.31 |
| 742581 | 1.20 | 0.36 | 3.04 | 4.31 | 3.03 | 3.03 | 3.14 | 3.31 |
| 811867 | 24.57 | 7.43 | 3.72 | 3.16 | 2.52 | 4.66 | 2.46 | 3.31 |
| 502622 | 18.65 | 5.65 | 2.32 | 2.97 | 3.27 | 5.80 | 2.16 | 3.30 |
| 666371 | 0.85 | 0.26 | 3.76 | 2.72 | 3.71 | 3.35 | 2.97 | 3.30 |
| 362829 | 3.03 | 0.92 | 3.18 | 5.12 | 3.27 | 3.18 | 1.73 | 3.30 |
| 129146 | 7.97 | 2.42 | 3.78 | 3.62 | 2.54 | 2.98 | 3.55 | 3.30 |
| 135083 | 17.07 | 5.19 | 3.09 | 3.47 | 4.93 | 2.81 | 2.15 | 3.29 |
| 854678 | 6.21 | 1.89 | 1.76 | 9.64 | 1.93 | 1.21 | 1.90 | 3.29 |
| 321470 | 7.74 | 2.36 | 3.41 | 4.28 | 3.25 | 1.89 | 3.60 | 3.29 |
| 1472638 | 7.11 | 2.16 | 2.45 | 3.79 | 2.73 | 4.52 | 2.93 | 3.29 |
| 242952 | 16.94 | 5.16 | 3.84 | 3.11 | 3.39 | 3.54 | 2.54 | 3.28 |
| 884657 | 6.68 | 2.04 | 3.32 | 2.87 | 2.28 | 4.95 | 2.99 | 3.28 |
| 40338 | 2.61 | 0.80 | 4.02 | 2.85 | 3.77 | 2.65 | 3.10 | 3.28 |
| 461670 | 8.90 | 2.71 | 2.03 | 4.16 | 4.85 | 2.37 | 2.99 | 3.28 |
| 45564 | 1.66 | 0.51 | 2.98 | 5.39 | 3.62 | 1.74 | 2.64 | 3.28 |
| 813637 | 34.08 | 10.41 | 3.65 | 3.22 | 2.20 | 5.54 | 1.75 | 3.27 |
| 291255 | 15.20 | 4.65 | 3.07 | 3.01 | 5.02 | 2.76 | 2.48 | 3.27 |
| 310493 | 7.56 | 2.32 | 3.61 | 3.65 | 3.06 | 2.77 | 3.24 | 3.26 |
| 433350 | 28.82 | 8.83 | 1.93 | 2.66 | 2.67 | 7.86 | 1.21 | 3.26 |
| 781454 | 9.75 | 2.99 | 2.79 | 1.30 | 9.94 | 1.22 | 1.06 | 3.26 |
| 685516 | 3.36 | 1.03 | 3.63 | 3.79 | 2.76 | 4.08 | 2.03 | 3.26 |
| 112785 | 7.75 | 2.38 | 2.21 | 3.83 | 3.58 | 3.82 | 2.81 | 3.25 |
| 264640 | 13.22 | 4.07 | 3.98 | 3.82 | 1.83 | 1.43 | 5.20 | 3.25 |
| 202904 | 12.77 | 3.93 | 2.44 | 4.34 | 2.65 | 3.34 | 3.48 | 3.25 |
| 251330 | 9.21 | 2.84 | 2.30 | 2.37 | 2.73 | 4.39 | 4.44 | 3.25 |
| 815164 | 0.88 | 0.27 | 3.76 | 3.41 | 4.07 | 3.68 | 1.32 | 3.25 |
| 1456602 | 6.11 | 1.88 | 2.40 | 2.64 | 2.44 | 4.72 | 4.03 | 3.25 |
| 1486260 | 13.23 | 4.08 | 2.75 | 2.87 | 3.17 | 5.07 | 2.36 | 3.24 |
| 322561 | 107.98 | 33.31 | 2.93 | 2.03 | 1.79 | 4.79 | 4.67 | 3.24 |
| 416436 | 11.08 | 3.42 | 5.36 | 3.73 | 2.74 | 3.15 | 1.23 | 3.24 |
| 827152 | 3.50 | 1.08 | 1.39 | 5.25 | 3.59 | 4.05 | 1.92 | 3.24 |
| 289818 | 7.47 | 2.31 | 2.49 | 4.60 | 3.59 | 3.30 | 2.19 | 3.23 |
| 273546 | 9.10 | 2.81 | 4.41 | 3.56 | 2.54 | 2.67 | 2.98 | 3.23 |
| 75886 | 14.08 | 4.37 | 5.08 | 3.68 | 1.09 | 4.18 | 2.11 | 3.23 |
| 416010 | 53.71 | 16.66 | 2.41 | 1.81 | 8.72 | 1.50 | 1.69 | 3.23 |
| 838628 | 2.82 | 0.88 | 2.12 | 3.44 | 3.70 | 3.20 | 3.67 | 3.23 |
| 22747 | 37.20 | 11.55 | 2.96 | 3.25 | 2.59 | 4.14 | 3.15 | 3.22 |
| 951091 | 2.03 | 0.63 | 2.71 | 3.17 | 4.10 | 2.80 | 3.30 | 3.22 |
| 35812 | 2.29 | 0.71 | 2.94 | 2.49 | 2.99 | 3.34 | 4.32 | 3.22 |
| 82874 | 5.14 | 1.60 | 4.36 | 3.56 | 2.21 | 3.84 | 2.10 | 3.21 |
| 1631713 | 5.68 | 1.77 | 2.81 | 3.64 | 3.90 | 2.77 | 2.93 | 3.21 |
| 263070 | 1.17 | 0.37 | 4.55 | 4.07 | 1.99 | 3.33 | 2.10 | 3.21 |
| 1031698 | 6.26 | 1.95 | 2.99 | 2.12 | 2.54 | 4.81 | 3.58 | 3.21 |
| 1602120 | 6.44 | 2.01 | 1.98 | 3.03 | 2.64 | 5.10 | 3.30 | 3.21 |
| 454672 | 7.68 | 2.40 | 5.39 | 3.57 | 1.75 | 2.15 | 3.16 | 3.20 |
| 837904 | 103.13 | 32.18 | 2.10 | 2.12 | 2.01 | 4.30 | 5.50 | 3.20 |
| 34869 | 2.74 | 0.85 | 1.85 | 2.96 | 1.76 | 7.56 | 1.87 | 3.20 |
| 840590 | 21.70 | 6.78 | 2.44 | 1.18 | 2.39 | 8.32 | 1.68 | 3.20 |
| 377672 | 2.59 | 0.81 | 0.93 | 0.98 | 0.68 | 12.46 | 0.96 | 3.20 |
| 511632 | 7.85 | 2.46 | 2.42 | 2.96 | 2.15 | 4.52 | 3.94 | 3.20 |
| 531028 | 21.14 | 6.62 | 3.62 | 3.65 | 3.05 | 2.41 | 3.26 | 3.20 |
| 321386 | 2.45 | 0.77 | 3.26 | 2.66 | 2.50 | 4.45 | 3.11 | 3.20 |
| 754221 | 6.80 | 2.13 | 2.49 | 3.95 | 5.19 | 2.42 | 1.93 | 3.20 |
| 433573 | 10.67 | 3.34 | 3.00 | 3.46 | 3.80 | 2.33 | 3.38 | 3.19 |
| 489823 | 33.49 | 10.49 | 2.75 | 4.23 | 1.58 | 5.36 | 2.05 | 3.19 |
| 248454 | 3.70 | 1.16 | 7.24 | 2.69 | 2.10 | 2.69 | 1.24 | 3.19 |
| 298155 | 2.85 | 0.89 | 2.52 | 4.31 | 2.59 | 3.88 | 2.66 | 3.19 |
| 530814 | 46.55 | 14.60 | 0.83 | 4.74 | 4.23 | 4.56 | 1.60 | 3.19 |
| 294259 | 4.21 | 1.32 | 3.87 | 2.34 | 3.12 | 1.09 | 5.52 | 3.19 |
| 726488 | 10.10 | 3.17 | 3.31 | 3.38 | 3.27 | 3.50 | 2.45 | 3.18 |
| 511459 | 37.04 | 11.64 | 2.30 | 3.42 | 4.76 | 3.38 | 2.06 | 3.18 |
| 503468 | 51.40 | 16.15 | 3.74 | 2.80 | 1.75 | 4.34 | 3.28 | 3.18 |
| 756600 | 47.06 | 14.79 | 3.37 | 1.74 | 1.79 | 4.21 | 4.80 | 3.18 |
| 760344 | 5.89 | 1.85 | 3.62 | 4.43 | 2.41 | 1.42 | 4.03 | 3.18 |
| 753252 | 7.78 | 2.45 | 2.52 | 2.78 | 2.96 | 4.05 | 3.59 | 3.18 |
| 26230 | 5.08 | 1.60 | 1.18 | 3.71 | 4.40 | 3.50 | 3.09 | 3.18 |
| 37980 | 0.84 | 0.27 | 1.41 | 2.73 | 1.73 | 8.06 | 1.95 | 3.18 |
| 156386 | 2.14 | 0.67 | 3.02 | 3.99 | 3.91 | 2.87 | 2.09 | 3.18 |
| 969844 | 9.12 | 2.88 | 5.15 | 2.28 | 2.19 | 3.92 | 2.33 | 3.17 |
| 855723 | 6.73 | 2.12 | 2.86 | 3.79 | 2.80 | 3.00 | 3.38 | 3.17 |
| 1240220 | 7.60 | 2.40 | 0.79 | 2.71 | 6.02 | 4.99 | 1.31 | 3.17 |
| 451769 | 5.25 | 1.66 | 3.67 | 2.29 | 2.52 | 5.57 | 1.78 | 3.16 |
| 810027 | 0.44 | 0.14 | 3.75 | 3.05 | 2.90 | 3.82 | 2.31 | 3.16 |
| 731272 | 5.02 | 1.59 | 2.45 | 2.89 | 3.95 | 4.89 | 1.62 | 3.16 |
| 129024 | 4.30 | 1.36 | 5.53 | 0.58 | 1.77 | 1.86 | 6.04 | 3.16 |
| 795907 | 5.39 | 1.71 | 1.43 | 1.64 | 3.37 | 0.61 | 8.72 | 3.15 |
| 731257 | 4.45 | 1.41 | 1.78 | 2.17 | 3.22 | 7.14 | 1.45 | 3.15 |
| 703707 | 4.12 | 1.31 | 2.68 | 2.65 | 3.51 | 3.30 | 3.63 | 3.15 |
| 811603 | 19.62 | 6.22 | 3.87 | 2.99 | 2.25 | 3.41 | 3.24 | 3.15 |
| 884346 | 5.38 | 1.71 | 2.20 | 4.78 | 3.45 | 3.15 | 2.15 | 3.15 |
| 201628 | 7.51 | 2.39 | 4.15 | 2.46 | 1.74 | 5.00 | 2.35 | 3.14 |
| 840821 | 17.59 | 5.60 | 3.31 | 3.10 | 3.72 | 2.88 | 2.71 | 3.14 |
| 344432 | 15.48 | 4.93 | 3.94 | 2.68 | 2.53 | 2.44 | 4.11 | 3.14 |
| 590853 | 0.66 | 0.21 | 2.61 | 2.78 | 3.43 | 3.40 | 3.47 | 3.14 |
| 767298 | 19.37 | 6.18 | 2.43 | 3.33 | 2.32 | 4.12 | 3.48 | 3.14 |
| 36387 | 3.19 | 1.02 | 2.66 | 3.88 | 4.50 | 1.19 | 3.46 | 3.14 |
| 302955 | 10.63 | 3.39 | 1.90 | 5.11 | 2.20 | 4.12 | 2.36 | 3.14 |
| 796885 | 9.64 | 3.08 | 4.15 | 1.95 | 2.26 | 4.54 | 2.77 | 3.13 |
| 725489 | 26.71 | 8.54 | 3.93 | 3.59 | 2.81 | 3.60 | 1.72 | 3.13 |
| 121489 | 34.70 | 11.09 | 3.79 | 3.88 | 3.08 | 2.54 | 2.35 | 3.13 |
| 263894 | 0.46 | 0.15 | 3.70 | 3.08 | 2.13 | 4.71 | 2.03 | 3.13 |
| 782383 | 1.32 | 0.42 | 2.40 | 2.83 | 2.35 | 5.64 | 2.42 | 3.13 |
| 815563 | 4.86 | 1.55 | 3.37 | 3.78 | 2.43 | 4.15 | 1.90 | 3.13 |
| 245547 | 5.86 | 1.88 | 1.15 | 5.91 | 3.40 | 3.50 | 1.66 | 3.12 |
| 856575 | 2.75 | 0.88 | 2.42 | 1.67 | 5.16 | 3.07 | 3.31 | 3.12 |
| 251753 | 2.29 | 0.74 | 2.40 | 2.73 | 2.75 | 5.27 | 2.45 | 3.12 |
| 356992 | 21.95 | 7.04 | 4.19 | 2.74 | 2.51 | 3.63 | 2.54 | 3.12 |
| 502463 | 5.77 | 1.85 | 2.78 | 3.60 | 3.10 | 2.63 | 3.49 | 3.12 |
| 1461528 | 3.95 | 1.27 | 3.05 | 2.21 | 5.36 | 2.64 | 2.33 | 3.12 |
| 244652 | 24.70 | 7.92 | 2.62 | 1.81 | 7.76 | 1.54 | 1.87 | 3.12 |
| 266106 | 20.68 | 6.63 | 2.76 | 3.21 | 6.30 | 1.40 | 1.93 | 3.12 |
| 1415981 | 4.13 | 1.32 | 3.58 | 3.02 | 4.59 | 2.33 | 2.07 | 3.12 |
| 66902 | 3.21 | 1.03 | 4.24 | 2.05 | 1.56 | 0.87 | 6.87 | 3.12 |
| 283919 | 7.56 | 2.43 | 7.31 | 3.63 | 1.47 | 2.02 | 1.15 | 3.11 |
| 813712 | 7.62 | 2.45 | 1.14 | 3.64 | 1.91 | 6.70 | 2.18 | 3.11 |
| 195903 | 6.95 | 2.24 | 2.45 | 4.56 | 3.51 | 2.97 | 2.05 | 3.11 |
| 345032 | 20.78 | 6.69 | 1.19 | 4.73 | 3.52 | 2.65 | 3.46 | 3.11 |
| 1635665 | 5.13 | 1.65 | 4.57 | 3.12 | 0.90 | 5.26 | 1.69 | 3.11 |
| 154749 | 1.76 | 0.57 | 3.34 | 3.27 | 3.77 | 2.57 | 2.58 | 3.11 |
| 287728 | 2.76 | 0.89 | 0.59 | 4.01 | 0.98 | 9.25 | 0.70 | 3.11 |
| 701409 | 1.54 | 0.50 | 4.21 | 2.63 | 3.95 | 2.39 | 2.34 | 3.10 |
| 627687 | 56.19 | 18.11 | 1.06 | 1.86 | 3.60 | 4.82 | 4.16 | 3.10 |
| 855244 | 15.78 | 5.09 | 6.04 | 0.82 | 7.22 | 0.56 | 0.88 | 3.10 |
| 293243 | 13.17 | 4.25 | 4.44 | 2.03 | 6.12 | 0.95 | 1.96 | 3.10 |
| 295206 | 3.16 | 1.02 | 5.72 | 3.11 | 1.43 | 2.48 | 2.76 | 3.10 |
| 744846 | 2.69 | 0.87 | 3.69 | 4.51 | 2.68 | 3.60 | 1.01 | 3.10 |
| 251645 | 0.50 | 0.16 | 3.12 | 3.21 | 3.74 | 3.30 | 2.12 | 3.10 |
| 357364 | 0.72 | 0.23 | 5.76 | 3.07 | 1.81 | 3.15 | 1.68 | 3.09 |
| 451707 | 7.59 | 2.46 | 2.46 | 1.83 | 6.35 | 1.34 | 3.48 | 3.09 |
| 250673 | 2.66 | 0.86 | 7.23 | 1.34 | 2.15 | 1.85 | 2.88 | 3.09 |
| 1031086 | 25.15 | 8.15 | 3.21 | 2.15 | 2.25 | 5.00 | 2.83 | 3.09 |
| 50892 | 7.95 | 2.58 | 3.62 | 2.53 | 3.85 | 2.69 | 2.71 | 3.08 |
| 52327 | 5.70 | 1.85 | 1.94 | 3.44 | 2.70 | 3.46 | 3.87 | 3.08 |
| 448930 | 7.53 | 2.45 | 2.47 | 2.25 | 2.41 | 3.62 | 4.66 | 3.08 |
| 713109 | 2.44 | 0.79 | 1.97 | 2.95 | 3.60 | 4.59 | 2.28 | 3.08 |
| 815794 | 14.69 | 4.77 | 5.65 | 1.93 | 1.67 | 1.95 | 4.18 | 3.08 |
| 789091 | 9.50 | 3.09 | 6.20 | 4.45 | 1.91 | 1.88 | 0.95 | 3.08 |
| 950836 | 34.79 | 11.31 | 2.78 | 1.53 | 8.20 | 0.92 | 1.95 | 3.08 |
| 213890 | 14.14 | 4.60 | 2.37 | 5.07 | 2.41 | 3.28 | 2.24 | 3.07 |
| 74223 | 5.42 | 1.76 | 3.29 | 3.47 | 3.41 | 2.68 | 2.52 | 3.07 |
| 41174 | 4.02 | 1.31 | 3.25 | 3.60 | 2.82 | 3.70 | 1.98 | 3.07 |
| 1635874 | 6.06 | 1.97 | 6.28 | 3.19 | 1.83 | 2.38 | 1.69 | 3.07 |
| 810284 | 2.27 | 0.74 | 5.17 | 2.15 | 2.18 | 2.13 | 3.71 | 3.07 |
| 845502 | 3.87 | 1.26 | 1.42 | 3.27 | 3.03 | 4.57 | 3.06 | 3.07 |
| 461489 | 0.92 | 0.30 | 3.19 | 3.40 | 2.48 | 2.98 | 3.28 | 3.07 |
| 361097 | 5.63 | 1.84 | 3.06 | 3.81 | 1.75 | 3.06 | 3.64 | 3.07 |
| 139113 | 4.32 | 1.41 | 2.39 | 3.80 | 2.88 | 5.14 | 1.10 | 3.06 |
| 813636 | 84.51 | 27.59 | 3.51 | 2.53 | 2.58 | 4.58 | 2.11 | 3.06 |
| 743804 | 5.09 | 1.66 | 3.74 | 2.59 | 2.21 | 3.39 | 3.38 | 3.06 |
| 486567 | 9.14 | 2.99 | 2.48 | 4.29 | 2.98 | 2.50 | 3.06 | 3.06 |
| 665127 | 5.48 | 1.79 | 3.90 | 3.70 | 2.31 | 2.65 | 2.73 | 3.06 |
| 136605 | 10.58 | 3.46 | 3.42 | 0.62 | 2.86 | 2.82 | 5.58 | 3.06 |
| 395625 | 14.45 | 4.73 | 2.38 | 3.22 | 3.22 | 3.25 | 3.22 | 3.06 |
| 287528 | 1.98 | 0.65 | 3.56 | 0.96 | 5.02 | 3.66 | 2.06 | 3.06 |
| 323474 | 1.25 | 0.41 | 5.34 | 3.25 | 2.05 | 2.36 | 2.27 | 3.06 |
| 884388 | 4.89 | 1.60 | 2.97 | 2.78 | 2.75 | 3.38 | 3.40 | 3.06 |
| 222527 | 2.88 | 0.94 | 1.68 | 4.03 | 3.69 | 4.09 | 1.79 | 3.05 |
| 260628 | 8.94 | 2.93 | 1.68 | 4.70 | 3.88 | 2.83 | 2.17 | 3.05 |
| 471266 | 2.72 | 0.89 | 3.15 | 2.95 | 2.66 | 3.45 | 3.03 | 3.05 |
| 565062 | 1.38 | 0.45 | 1.72 | 3.15 | 3.97 | 2.87 | 3.53 | 3.05 |
| 701532 | 3.26 | 1.07 | 3.06 | 2.86 | 3.51 | 1.98 | 3.84 | 3.05 |
| 878127 | 3.53 | 1.16 | 2.07 | 3.35 | 4.53 | 3.32 | 1.96 | 3.05 |
| 878373 | 31.06 | 10.19 | 3.07 | 3.84 | 2.03 | 4.00 | 2.29 | 3.05 |
| 40075 | 3.13 | 1.03 | 2.09 | 3.44 | 2.70 | 5.00 | 2.00 | 3.05 |
| 884766 | 9.95 | 3.27 | 1.22 | 3.47 | 2.45 | 2.67 | 5.40 | 3.04 |
| 754192 | 3.30 | 1.09 | 4.21 | 2.70 | 2.65 | 3.45 | 2.21 | 3.04 |
| 299154 | 1.37 | 0.45 | 1.64 | 3.95 | 3.01 | 2.17 | 4.42 | 3.04 |
| 344589 | 53.28 | 17.55 | 1.28 | 4.11 | 3.84 | 2.63 | 3.31 | 3.04 |
| 897153 | 10.77 | 3.55 | 3.27 | 3.89 | 4.14 | 2.12 | 1.76 | 3.04 |
| 328613 | 0.46 | 0.15 | 2.60 | 3.52 | 3.31 | 2.33 | 3.42 | 3.03 |
| 884867 | 24.71 | 8.14 | 3.57 | 3.43 | 1.95 | 3.39 | 2.83 | 3.03 |
| 289288 | 5.44 | 1.79 | 3.47 | 2.18 | 3.94 | 1.96 | 3.62 | 3.03 |
| 788518 | 13.97 | 4.61 | 2.21 | 4.58 | 2.41 | 3.19 | 2.78 | 3.03 |
| 825842 | 6.00 | 1.98 | 1.78 | 2.66 | 4.19 | 1.45 | 5.08 | 3.03 |
| 133130 | 11.12 | 3.67 | 3.58 | 0.72 | 2.94 | 2.70 | 5.20 | 3.03 |
| 252864 | 1.20 | 0.40 | 2.46 | 3.75 | 2.39 | 3.50 | 3.03 | 3.03 |
| 813187 | 22.70 | 7.51 | 1.31 | 2.52 | 4.98 | 3.97 | 2.34 | 3.02 |
| 1635110 | 38.13 | 12.62 | 4.50 | 2.40 | 3.02 | 2.46 | 2.73 | 3.02 |
| 266747 | 3.05 | 1.01 | 3.26 | 3.57 | 2.70 | 0.99 | 4.59 | 3.02 |
| 490360 | 5.94 | 1.97 | 2.87 | 3.33 | 2.69 | 3.50 | 2.72 | 3.02 |
| 951216 | 2.02 | 0.67 | 3.88 | 2.20 | 2.08 | 4.19 | 2.75 | 3.02 |
| 322914 | 9.34 | 3.10 | 4.67 | 3.02 | 2.07 | 1.93 | 3.39 | 3.02 |
| 845609 | 15.29 | 5.07 | 3.85 | 3.88 | 2.84 | 3.05 | 1.46 | 3.02 |
| 1467108 | 4.29 | 1.42 | 1.21 | 3.61 | 4.50 | 3.23 | 2.53 | 3.02 |
| 415978 | 24.58 | 8.16 | 2.76 | 1.42 | 3.19 | 4.93 | 2.76 | 3.01 |
| 645368 | 8.48 | 2.82 | 2.47 | 3.08 | 3.32 | 2.42 | 3.76 | 3.01 |
| 854899 | 18.74 | 6.24 | 3.77 | 2.48 | 2.61 | 2.49 | 3.67 | 3.00 |
| 80764 | 26.16 | 8.72 | 2.38 | 2.68 | 2.24 | 3.93 | 3.77 | 3.00 |
| 1501998 | 2.03 | 0.68 | 3.20 | 3.50 | 2.70 | 3.57 | 2.05 | 3.00 |
| 590298 | 10.43 | 3.47 | 3.90 | 3.96 | 3.21 | 2.04 | 1.90 | 3.00 |
| 594693 | 7.33 | 2.44 | 3.84 | 2.11 | 7.17 | 1.01 | 0.88 | 3.00 |
| 856115 | 1.30 | 0.43 | 9.13 | 1.80 | 2.06 | 0.91 | 1.10 | 3.00 |
| 364022 | 4.15 | 1.39 | 3.12 | 5.09 | 2.52 | 2.61 | 1.64 | 3.00 |
| 730412 | 1.25 | 0.42 | 2.86 | 2.00 | 3.95 | 3.63 | 2.55 | 3.00 |
| 843140 | 14.94 | 4.99 | 1.67 | 3.33 | 3.94 | 2.97 | 3.06 | 3.00 |
| 342211 | 1.92 | 0.64 | 2.75 | 3.24 | 2.28 | 4.81 | 1.90 | 3.00 |
| 742806 | 1.76 | 0.59 | 2.37 | 3.25 | 4.16 | 2.33 | 2.85 | 2.99 |
| 884480 | 11.68 | 3.91 | 3.07 | 2.74 | 1.85 | 4.27 | 3.01 | 2.99 |
| 1636707 | 19.22 | 6.44 | 2.58 | 3.23 | 2.07 | 3.98 | 3.07 | 2.99 |
| 712610 | 1.06 | 0.35 | 3.02 | 3.36 | 3.85 | 3.46 | 1.23 | 2.98 |
| 360232 | 2.20 | 0.74 | 1.98 | 3.91 | 3.63 | 3.66 | 1.72 | 2.98 |
| 74511 | 5.23 | 1.75 | 4.10 | 3.44 | 2.52 | 0.97 | 3.89 | 2.98 |
| 343167 | 15.62 | 5.24 | 3.15 | 4.46 | 2.96 | 2.05 | 2.27 | 2.98 |
| 39885 | 0.94 | 0.31 | 0.88 | 2.80 | 4.66 | 4.76 | 1.80 | 2.98 |
| 122183 | 7.61 | 2.56 | 2.66 | 4.22 | 2.01 | 2.53 | 3.46 | 2.98 |
| 38253 | 2.80 | 0.94 | 3.19 | 4.23 | 2.69 | 2.79 | 1.98 | 2.98 |
| 745103 | 8.87 | 2.98 | 2.03 | 3.44 | 3.39 | 2.05 | 3.96 | 2.97 |
| 155838 | 2.53 | 0.85 | 2.70 | 1.94 | 5.31 | 2.18 | 2.74 | 2.97 |
| 813149 | 6.11 | 2.06 | 5.57 | 3.70 | 2.01 | 2.38 | 1.21 | 2.97 |
| 469685 | 4.95 | 1.67 | 3.25 | 3.34 | 2.23 | 3.52 | 2.51 | 2.97 |
| 824640 | 6.96 | 2.35 | 1.37 | 1.54 | 3.69 | 3.65 | 4.59 | 2.97 |
| 825833 | 1.95 | 0.66 | 3.29 | 2.75 | 4.19 | 2.65 | 1.97 | 2.97 |
| 25517 | 8.52 | 2.88 | 3.15 | 3.29 | 2.67 | 2.43 | 3.28 | 2.97 |
| 842767 | 2.15 | 0.73 | 2.59 | 3.85 | 3.19 | 2.67 | 2.53 | 2.96 |
| 148225 | 3.89 | 1.31 | 2.29 | 2.66 | 1.88 | 5.50 | 2.47 | 2.96 |
| 772304 | 113.14 | 38.24 | 3.78 | 2.19 | 2.18 | 2.83 | 3.83 | 2.96 |
| 246800 | 6.67 | 2.26 | 2.06 | 3.22 | 2.26 | 4.33 | 2.91 | 2.96 |
| 322192 | 7.97 | 2.69 | 1.72 | 3.28 | 5.02 | 1.74 | 3.02 | 2.96 |
| 755765 | 4.01 | 1.36 | 2.43 | 3.91 | 4.37 | 1.46 | 2.62 | 2.96 |
| 462468 | 3.24 | 1.10 | 3.23 | 3.74 | 3.40 | 1.72 | 2.69 | 2.96 |
| 701402 | 4.23 | 1.43 | 1.82 | 3.11 | 5.63 | 2.91 | 1.31 | 2.96 |
| 253253 | 1.73 | 0.59 | 4.71 | 4.49 | 1.70 | 2.17 | 1.68 | 2.95 |
| 211206 | 4.71 | 1.60 | 2.58 | 3.21 | 2.52 | 2.63 | 3.82 | 2.95 |
| 813825 | 0.52 | 0.18 | 3.85 | 3.04 | 1.69 | 4.84 | 1.32 | 2.95 |
| 898227 | 2.02 | 0.69 | 1.45 | 4.11 | 2.91 | 4.09 | 2.18 | 2.95 |
| 45578 | 5.05 | 1.72 | 4.61 | 2.16 | 2.26 | 3.42 | 2.27 | 2.94 |
| 1522799 | 1.23 | 0.42 | 2.25 | 3.40 | 2.72 | 3.47 | 2.86 | 2.94 |
| 593520 | 6.25 | 2.13 | 2.05 | 2.67 | 3.01 | 4.62 | 2.34 | 2.94 |
| 487773 | 29.51 | 10.06 | 4.78 | 2.56 | 4.05 | 1.34 | 1.95 | 2.93 |
| 502690 | 4.09 | 1.39 | 2.22 | 3.28 | 2.51 | 4.37 | 2.29 | 2.93 |
| 731104 | 0.65 | 0.22 | 3.42 | 2.35 | 3.12 | 2.70 | 3.07 | 2.93 |
| 796341 | 7.16 | 2.44 | 2.21 | 2.93 | 2.48 | 3.55 | 3.49 | 2.93 |
| 1492304 | 4.23 | 1.44 | 2.24 | 2.81 | 3.19 | 4.05 | 2.36 | 2.93 |
| 77805 | 15.55 | 5.31 | 2.32 | 4.51 | 2.41 | 2.27 | 3.13 | 2.93 |
| 415700 | 3.36 | 1.15 | 2.29 | 2.89 | 4.79 | 1.53 | 3.14 | 2.93 |
| 701496 | 2.28 | 0.78 | 2.43 | 2.22 | 5.16 | 3.20 | 1.62 | 2.93 |
| 233419 | 5.55 | 1.90 | 1.88 | 3.96 | 3.98 | 1.76 | 3.06 | 2.93 |
| 767994 | 4.29 | 1.47 | 2.76 | 1.52 | 8.75 | 0.69 | 0.92 | 2.93 |
| 254310 | 10.26 | 3.51 | 3.10 | 2.50 | 6.68 | 1.35 | 1.00 | 2.92 |
| 48033 | 0.51 | 0.18 | 2.77 | 3.62 | 3.02 | 2.44 | 2.76 | 2.92 |
| 811842 | 9.91 | 3.39 | 2.78 | 3.26 | 2.72 | 2.81 | 3.05 | 2.92 |
| 44975 | 8.91 | 3.05 | 2.47 | 4.15 | 2.22 | 2.69 | 3.08 | 2.92 |
| 243068 | 9.34 | 3.20 | 2.27 | 2.18 | 2.15 | 5.77 | 2.23 | 2.92 |
| 1635350 | 10.17 | 3.48 | 2.60 | 2.63 | 5.83 | 2.20 | 1.33 | 2.92 |
| 759948 | 4.05 | 1.39 | 2.12 | 1.92 | 1.94 | 7.00 | 1.59 | 2.91 |
| 35807 | 2.91 | 1.00 | 3.30 | 2.96 | 2.26 | 3.58 | 2.45 | 2.91 |
| 45515 | 3.88 | 1.33 | 1.58 | 3.77 | 5.25 | 1.91 | 2.04 | 2.91 |
| 1591622 | 49.35 | 16.97 | 2.27 | 3.52 | 1.93 | 4.64 | 2.17 | 2.91 |
| 460841 | 1.02 | 0.35 | 4.19 | 1.78 | 4.04 | 2.42 | 2.11 | 2.91 |
| 1411726 | 30.68 | 10.55 | 3.69 | 2.02 | 2.19 | 4.86 | 1.78 | 2.91 |
| 160485 | 5.17 | 1.78 | 3.02 | 1.25 | 1.91 | 2.64 | 5.72 | 2.91 |
| 742695 | 1.83 | 0.63 | 2.56 | 3.08 | 2.90 | 4.14 | 1.83 | 2.90 |
| 242706 | 5.15 | 1.78 | 3.05 | 3.96 | 1.69 | 3.81 | 1.99 | 2.90 |
| 868838 | 3.29 | 1.14 | 1.07 | 1.24 | 0.96 | 9.00 | 2.21 | 2.90 |
| 1420830 | 3.06 | 1.06 | 1.37 | 2.47 | 2.52 | 4.60 | 3.53 | 2.90 |
| 825771 | 12.13 | 4.19 | 2.16 | 5.30 | 2.82 | 2.12 | 2.09 | 2.90 |
| 826102 | 4.19 | 1.45 | 2.02 | 1.86 | 7.90 | 1.13 | 1.56 | 2.89 |
| 461488 | 0.55 | 0.19 | 3.29 | 3.33 | 2.51 | 2.75 | 2.59 | 2.89 |
| 49485 | 0.54 | 0.19 | 3.81 | 2.49 | 2.73 | 3.24 | 2.15 | 2.89 |
| 129725 | 17.94 | 6.22 | 2.93 | 1.99 | 4.17 | 3.16 | 2.17 | 2.89 |
| 627273 | 1.58 | 0.55 | 1.86 | 3.85 | 2.31 | 3.67 | 2.73 | 2.88 |
| 143306 | 10.42 | 3.62 | 3.88 | 2.52 | 1.98 | 3.46 | 2.55 | 2.88 |
| 730313 | 3.95 | 1.37 | 2.62 | 2.27 | 3.50 | 2.08 | 3.93 | 2.88 |
| 825718 | 4.03 | 1.40 | 2.44 | 2.50 | 3.77 | 3.02 | 2.67 | 2.88 |
| 1591264 | 3.18 | 1.11 | 4.68 | 4.17 | 2.19 | 2.11 | 1.23 | 2.88 |
| 784016 | 33.80 | 11.75 | 2.08 | 4.19 | 3.69 | 2.77 | 1.66 | 2.88 |
| 41940 | 10.51 | 3.65 | 1.79 | 2.81 | 3.34 | 3.64 | 2.80 | 2.88 |
| 841501 | 5.94 | 2.07 | 3.74 | 4.22 | 2.48 | 2.62 | 1.31 | 2.88 |
| 853968 | 12.10 | 4.21 | 3.42 | 3.85 | 5.08 | 1.19 | 0.84 | 2.87 |
| 489663 | 2.11 | 0.73 | 3.88 | 2.51 | 1.67 | 3.15 | 3.17 | 2.87 |
| 669371 | 4.99 | 1.74 | 2.07 | 1.98 | 7.30 | 1.15 | 1.87 | 2.87 |
| 815760 | 2.31 | 0.81 | 3.18 | 2.00 | 4.13 | 3.96 | 1.09 | 2.87 |
| 125722 | 3.28 | 1.14 | 4.52 | 2.27 | 2.75 | 3.95 | 0.86 | 2.87 |
| 897625 | 2.58 | 0.90 | 1.23 | 2.12 | 4.08 | 3.14 | 3.79 | 2.87 |
| 768179 | 3.80 | 1.32 | 1.92 | 3.06 | 2.43 | 4.11 | 2.83 | 2.87 |
| 768469 | 4.17 | 1.45 | 2.78 | 1.73 | 2.41 | 3.64 | 3.79 | 2.87 |
| 898265 | 20.16 | 7.03 | 1.31 | 2.31 | 3.96 | 2.43 | 4.34 | 2.87 |
| 868119 | 10.00 | 3.49 | 2.81 | 3.31 | 3.66 | 1.82 | 2.73 | 2.87 |
| 897983 | 26.99 | 9.42 | 2.98 | 3.14 | 2.24 | 2.33 | 3.64 | 2.86 |
| 1416086 | 8.24 | 2.88 | 3.59 | 2.09 | 5.52 | 1.57 | 1.54 | 2.86 |
| 141972 | 18.88 | 6.60 | 3.58 | 3.00 | 3.07 | 1.99 | 2.68 | 2.86 |
| 144977 | 71.81 | 25.09 | 2.67 | 3.73 | 3.36 | 1.42 | 3.14 | 2.86 |
| 949940 | 12.52 | 4.38 | 4.69 | 2.75 | 1.41 | 3.20 | 2.25 | 2.86 |
| 898251 | 2.02 | 0.71 | 2.79 | 2.93 | 3.12 | 4.01 | 1.45 | 2.86 |
| 80500 | 140.81 | 49.33 | 0.91 | 2.65 | 1.64 | 5.24 | 3.85 | 2.86 |
| 823588 | 7.72 | 2.70 | 2.44 | 3.65 | 3.22 | 2.51 | 2.45 | 2.85 |
| 269300 | 10.64 | 3.73 | 1.47 | 2.55 | 4.46 | 4.07 | 1.71 | 2.85 |
| 823627 | 3.25 | 1.14 | 2.72 | 3.08 | 3.03 | 1.90 | 3.53 | 2.85 |
| 1589973 | 5.17 | 1.81 | 2.56 | 3.21 | 1.99 | 4.51 | 1.99 | 2.85 |
| 246541 | 10.77 | 3.78 | 2.76 | 3.03 | 2.41 | 3.92 | 2.14 | 2.85 |
| 784253 | 11.76 | 4.12 | 2.36 | 3.67 | 2.76 | 2.44 | 3.04 | 2.85 |
| 27072 | 0.29 | 0.10 | 2.61 | 2.23 | 3.44 | 2.93 | 3.04 | 2.85 |
| 754449 | 1.78 | 0.63 | 2.10 | 3.63 | 2.52 | 4.00 | 1.99 | 2.85 |
| 82905 | 1.21 | 0.43 | 3.27 | 1.88 | 1.90 | 3.49 | 3.70 | 2.85 |
| 743828 | 3.34 | 1.17 | 1.16 | 1.69 | 5.22 | 0.99 | 5.18 | 2.85 |
| 743851 | 8.22 | 2.89 | 3.40 | 2.79 | 2.54 | 3.77 | 1.72 | 2.84 |
| 1475746 | 12.48 | 4.39 | 3.24 | 2.13 | 2.29 | 3.86 | 2.70 | 2.84 |
| 283196 | 3.80 | 1.34 | 1.51 | 2.93 | 1.19 | 6.19 | 2.38 | 2.84 |
| 866276 | 1.94 | 0.68 | 1.99 | 3.31 | 2.96 | 2.62 | 3.33 | 2.84 |
| 144926 | 9.20 | 3.24 | 3.79 | 2.02 | 2.76 | 3.87 | 1.75 | 2.84 |
| 769926 | 9.93 | 3.51 | 6.19 | 1.95 | 2.54 | 2.39 | 1.09 | 2.83 |
| 730554 | 11.89 | 4.20 | 2.36 | 3.04 | 1.33 | 4.78 | 2.64 | 2.83 |
| 40017 | 8.29 | 2.94 | 2.97 | 3.32 | 1.84 | 3.86 | 2.12 | 2.82 |
| 897577 | 0.52 | 0.18 | 2.18 | 2.74 | 3.06 | 3.81 | 2.31 | 2.82 |
| 1492412 | 236.26 | 83.73 | 1.73 | 2.18 | 3.44 | 2.84 | 3.92 | 2.82 |
| 1336262 | 35.80 | 12.69 | 4.41 | 2.34 | 4.62 | 1.32 | 1.41 | 2.82 |
| 250678 | 4.24 | 1.51 | 3.14 | 3.84 | 3.52 | 1.72 | 1.86 | 2.82 |
| 810220 | 2.17 | 0.77 | 4.33 | 3.00 | 1.34 | 3.50 | 1.92 | 2.82 |
| 271076 | 4.31 | 1.53 | 2.34 | 3.42 | 3.53 | 2.75 | 2.04 | 2.82 |
| 111812 | 5.56 | 1.98 | 3.30 | 4.08 | 4.25 | 1.15 | 1.29 | 2.81 |
| 898096 | 4.06 | 1.45 | 3.41 | 2.02 | 3.72 | 1.08 | 3.83 | 2.81 |
| 250869 | 123.92 | 44.10 | 8.11 | 2.05 | 1.68 | 0.76 | 1.46 | 2.81 |
| 840620 | 13.81 | 4.92 | 1.46 | 1.93 | 2.95 | 3.21 | 4.47 | 2.81 |
| 53165 | 0.89 | 0.32 | 3.53 | 3.16 | 2.83 | 2.33 | 2.16 | 2.80 |
| 784128 | 1.83 | 0.65 | 1.61 | 1.75 | 6.85 | 1.95 | 1.84 | 2.80 |
| 452668 | 4.95 | 1.77 | 2.13 | 1.80 | 2.80 | 5.84 | 1.44 | 2.80 |
| 811911 | 11.89 | 4.25 | 3.15 | 1.99 | 3.02 | 2.42 | 3.41 | 2.80 |
| 854122 | 4.20 | 1.50 | 5.92 | 3.38 | 2.72 | 0.79 | 1.18 | 2.80 |
| 491184 | 6.35 | 2.27 | 3.28 | 1.15 | 8.07 | 0.69 | 0.80 | 2.80 |
| 825356 | 1.18 | 0.42 | 2.48 | 2.51 | 4.19 | 3.57 | 1.22 | 2.79 |
| 214884 | 3.55 | 1.27 | 4.42 | 2.54 | 1.68 | 3.83 | 1.50 | 2.79 |
| 450711 | 1.29 | 0.46 | 2.21 | 3.64 | 2.22 | 4.08 | 1.82 | 2.79 |
| 1593502 | 2.25 | 0.81 | 1.62 | 3.22 | 2.57 | 2.01 | 4.54 | 2.79 |
| 825058 | 3.42 | 1.23 | 2.71 | 1.17 | 8.02 | 1.37 | 0.68 | 2.79 |
| 285226 | 25.74 | 9.23 | 7.63 | 1.20 | 2.23 | 1.77 | 1.12 | 2.79 |
| 191904 | 7.67 | 2.75 | 3.12 | 2.55 | 2.70 | 2.27 | 3.28 | 2.79 |
| 786609 | 4.81 | 1.73 | 0.70 | 0.71 | 2.56 | 9.16 | 0.80 | 2.78 |
| 204146 | 2.40 | 0.86 | 1.54 | 5.79 | 2.84 | 1.41 | 2.34 | 2.78 |
| 713286 | 0.89 | 0.32 | 3.32 | 2.65 | 3.34 | 3.52 | 1.08 | 2.78 |
| 416833 | 6.07 | 2.18 | 1.35 | 3.81 | 1.74 | 4.90 | 2.09 | 2.78 |
| 268211 | 3.43 | 1.23 | 2.33 | 1.79 | 1.77 | 1.26 | 6.74 | 2.78 |
| 782439 | 35.89 | 12.92 | 3.09 | 2.38 | 1.70 | 3.98 | 2.74 | 2.78 |
| 810741 | 72.43 | 26.10 | 3.64 | 2.64 | 2.20 | 3.00 | 2.38 | 2.78 |
| 431286 | 13.81 | 4.98 | 2.22 | 2.98 | 3.24 | 1.94 | 3.50 | 2.77 |
| 826335 | 2.24 | 0.81 | 2.09 | 2.46 | 3.66 | 3.16 | 2.49 | 2.77 |
| 796313 | 0.78 | 0.28 | 3.22 | 2.69 | 2.55 | 2.13 | 3.26 | 2.77 |
| 812989 | 2.18 | 0.79 | 2.58 | 2.17 | 3.23 | 3.95 | 1.93 | 2.77 |
| 258521 | 3.33 | 1.20 | 1.48 | 7.97 | 1.12 | 1.13 | 2.14 | 2.77 |
| 526945 | 1.14 | 0.41 | 3.17 | 1.74 | 2.47 | 3.11 | 3.36 | 2.77 |
| 197821 | 5.95 | 2.15 | 5.83 | 4.39 | 1.04 | 1.29 | 1.28 | 2.77 |
| 1631747 | 5.09 | 1.84 | 4.14 | 4.87 | 1.99 | 1.76 | 1.08 | 2.77 |
| 1475738 | 238.01 | 86.12 | 3.33 | 2.08 | 2.23 | 3.93 | 2.25 | 2.76 |
| 248261 | 1.51 | 0.55 | 4.72 | 1.35 | 0.99 | 1.14 | 5.61 | 2.76 |
| 841348 | 11.96 | 4.33 | 4.50 | 2.03 | 0.87 | 4.69 | 1.72 | 2.76 |
| 1031346 | 3.21 | 1.16 | 3.61 | 2.85 | 2.26 | 3.16 | 1.91 | 2.76 |
| 244154 | 4.94 | 1.79 | 2.49 | 2.18 | 3.84 | 2.06 | 3.22 | 2.76 |
| 245860 | 30.36 | 11.02 | 5.36 | 1.24 | 5.40 | 0.69 | 1.09 | 2.76 |
| 271472 | 11.80 | 4.28 | 2.07 | 3.57 | 2.66 | 2.40 | 3.07 | 2.76 |
| 753917 | 6.49 | 2.36 | 2.95 | 3.41 | 1.98 | 3.47 | 1.95 | 2.75 |
| 51749 | 3.65 | 1.33 | 2.28 | 1.01 | 1.83 | 2.34 | 6.28 | 2.75 |
| 296429 | 5.99 | 2.18 | 1.94 | 3.44 | 2.46 | 2.47 | 3.43 | 2.75 |
| 430318 | 10.87 | 3.96 | 1.77 | 2.79 | 3.60 | 1.97 | 3.59 | 2.74 |
| 283142 | 3.58 | 1.31 | 3.20 | 3.03 | 3.74 | 1.95 | 1.78 | 2.74 |
| 435341 | 1.73 | 0.63 | 3.20 | 2.13 | 2.58 | 3.61 | 2.19 | 2.74 |
| 278531 | 57.48 | 20.99 | 1.66 | 3.41 | 1.66 | 4.44 | 2.52 | 2.74 |
| 489722 | 4.03 | 1.47 | 2.33 | 2.25 | 3.98 | 3.06 | 2.07 | 2.74 |
| 814945 | 4.97 | 1.82 | 2.62 | 3.11 | 2.36 | 3.47 | 2.14 | 2.74 |
| 503741 | 3.64 | 1.33 | 1.17 | 3.06 | 1.97 | 5.66 | 1.82 | 2.74 |
| 1632141 | 2.15 | 0.79 | 2.34 | 3.20 | 3.06 | 2.96 | 2.13 | 2.74 |
| 428215 | 42.11 | 15.40 | 3.87 | 3.41 | 1.92 | 2.28 | 2.19 | 2.74 |
| 878316 | 5.34 | 1.95 | 2.19 | 2.60 | 1.52 | 4.16 | 3.20 | 2.73 |
| 1573087 | 2.41 | 0.88 | 1.63 | 2.09 | 4.06 | 4.16 | 1.72 | 2.73 |
| 841399 | 9.08 | 3.33 | 2.44 | 2.07 | 3.48 | 3.21 | 2.45 | 2.73 |
| 897730 | 41.12 | 15.07 | 3.07 | 1.82 | 2.32 | 2.54 | 3.88 | 2.73 |
| 866882 | 10.73 | 3.94 | 1.81 | 4.01 | 3.71 | 1.83 | 2.28 | 2.73 |
| 1160558 | 8.03 | 2.94 | 2.15 | 3.29 | 2.53 | 3.66 | 2.00 | 2.73 |
| 127928 | 3.67 | 1.35 | 1.79 | 3.28 | 2.89 | 2.15 | 3.51 | 2.73 |
| 897971 | 11.55 | 4.24 | 1.43 | 2.82 | 3.44 | 2.74 | 3.21 | 2.73 |
| 447568 | 9.00 | 3.31 | 3.49 | 1.70 | 1.86 | 4.48 | 2.08 | 2.72 |
| 811880 | 3.13 | 1.15 | 1.85 | 2.58 | 4.75 | 3.12 | 1.30 | 2.72 |
| 810725 | 3.27 | 1.20 | 1.92 | 2.23 | 1.63 | 5.97 | 1.85 | 2.72 |
| 197176 | 20.79 | 7.65 | 3.06 | 2.51 | 3.48 | 2.16 | 2.38 | 2.72 |
| 758347 | 2.75 | 1.01 | 1.61 | 3.59 | 3.19 | 2.80 | 2.41 | 2.72 |
| 43705 | 0.35 | 0.13 | 2.72 | 3.74 | 1.90 | 3.12 | 2.09 | 2.71 |
| 813280 | 4.14 | 1.53 | 1.72 | 2.58 | 2.29 | 3.06 | 3.91 | 2.71 |
| 195652 | 18.03 | 6.65 | 2.18 | 3.01 | 2.07 | 4.00 | 2.29 | 2.71 |
| 868308 | 65.99 | 24.35 | 3.48 | 1.83 | 1.37 | 4.88 | 2.00 | 2.71 |
| 429906 | 5.35 | 1.98 | 1.98 | 3.10 | 2.51 | 2.18 | 3.77 | 2.71 |
| 83011 | 32.97 | 12.18 | 2.39 | 2.09 | 2.06 | 3.35 | 3.64 | 2.71 |
| 838611 | 52.82 | 19.51 | 4.00 | 0.88 | 1.23 | 4.84 | 2.60 | 2.71 |
| 783697 | 3.19 | 1.18 | 2.47 | 3.13 | 3.04 | 2.62 | 2.26 | 2.70 |
| 268178 | 28.02 | 10.37 | 1.92 | 3.23 | 2.46 | 3.05 | 2.85 | 2.70 |
| 460487 | 24.08 | 8.92 | 0.68 | 0.85 | 5.68 | 0.08 | 6.21 | 2.70 |
| 418053 | 7.57 | 2.80 | 3.25 | 1.89 | 2.22 | 3.44 | 2.70 | 2.70 |
| 884894 | 41.65 | 15.44 | 3.11 | 2.60 | 1.96 | 3.14 | 2.68 | 2.70 |
| 824066 | 4.08 | 1.51 | 2.48 | 2.33 | 1.91 | 5.02 | 1.74 | 2.70 |
| 950450 | 1.77 | 0.66 | 1.96 | 2.45 | 3.15 | 3.54 | 2.39 | 2.70 |
| 50344 | 0.85 | 0.32 | 1.00 | 1.06 | 0.91 | 9.45 | 1.06 | 2.70 |
| 42076 | 5.72 | 2.12 | 2.14 | 3.48 | 2.38 | 2.44 | 3.04 | 2.69 |
| 68636 | 4.83 | 1.80 | 2.78 | 3.27 | 2.43 | 3.37 | 1.62 | 2.69 |
| 26185 | 1.15 | 0.43 | 1.89 | 2.57 | 3.38 | 3.81 | 1.78 | 2.69 |
| 278687 | 13.10 | 4.88 | 2.06 | 4.03 | 2.38 | 1.89 | 3.07 | 2.69 |
| 391984 | 4.56 | 1.70 | 3.22 | 3.44 | 1.67 | 2.03 | 3.06 | 2.68 |
| 279785 | 0.91 | 0.34 | 1.97 | 1.94 | 1.70 | 5.89 | 1.92 | 2.68 |
| 897199 | 14.78 | 5.51 | 3.48 | 2.55 | 2.61 | 3.07 | 1.71 | 2.68 |
| 263697 | 4.23 | 1.58 | 3.06 | 3.23 | 4.13 | 1.12 | 1.87 | 2.68 |
| 770983 | 7.24 | 2.70 | 1.30 | 5.12 | 2.60 | 2.36 | 2.03 | 2.68 |
| 505344 | 2.26 | 0.84 | 3.69 | 2.24 | 1.96 | 3.13 | 2.38 | 2.68 |
| 253865 | 1.68 | 0.63 | 5.25 | 3.14 | 1.49 | 1.55 | 1.93 | 2.67 |
| 752690 | 6.05 | 2.27 | 3.98 | 2.14 | 0.98 | 4.23 | 2.02 | 2.67 |
| 857681 | 54.39 | 20.36 | 3.42 | 2.88 | 1.57 | 3.42 | 2.07 | 2.67 |
| 1049033 | 3.07 | 1.15 | 2.50 | 3.09 | 3.57 | 3.21 | 0.99 | 2.67 |
| 743041 | 6.48 | 2.43 | 3.55 | 3.05 | 3.00 | 2.00 | 1.76 | 2.67 |
| 461761 | 8.65 | 3.24 | 2.63 | 1.55 | 4.32 | 3.11 | 1.72 | 2.67 |
| 51921 | 4.48 | 1.68 | 5.04 | 1.90 | 2.84 | 1.30 | 2.26 | 2.67 |
| 1471829 | 99.54 | 37.33 | 2.97 | 2.21 | 1.62 | 5.24 | 1.30 | 2.67 |
| 307882 | 7.09 | 2.66 | 3.13 | 1.77 | 2.17 | 4.14 | 2.12 | 2.67 |
| 743081 | 3.75 | 1.41 | 4.46 | 3.27 | 1.20 | 2.05 | 2.35 | 2.67 |
| 251452 | 3.12 | 1.17 | 1.29 | 2.18 | 2.99 | 5.63 | 1.24 | 2.67 |
| 795738 | 2.06 | 0.77 | 3.12 | 3.28 | 1.60 | 3.79 | 1.52 | 2.66 |
| 838478 | 2.51 | 0.94 | 4.43 | 1.79 | 2.84 | 1.08 | 3.16 | 2.66 |
| 361323 | 4.53 | 1.70 | 1.66 | 1.41 | 2.10 | 6.37 | 1.76 | 2.66 |
| 529861 | 5.75 | 2.16 | 2.44 | 2.09 | 3.05 | 3.43 | 2.29 | 2.66 |
| 486175 | 10.39 | 3.91 | 1.74 | 7.19 | 2.53 | 0.92 | 0.91 | 2.66 |
| 307029 | 56.38 | 21.22 | 3.05 | 1.98 | 1.59 | 4.58 | 2.09 | 2.66 |
| 243321 | 2.79 | 1.05 | 2.81 | 1.53 | 1.43 | 1.74 | 5.74 | 2.65 |
| 590398 | 12.27 | 4.63 | 4.11 | 2.22 | 2.03 | 3.29 | 1.58 | 2.65 |
| 1475028 | 249.73 | 94.34 | 2.17 | 1.81 | 1.81 | 4.31 | 3.14 | 2.65 |
| 595604 | 9.41 | 3.56 | 1.72 | 1.96 | 4.39 | 3.06 | 2.10 | 2.65 |
| 188151 | 3.25 | 1.23 | 1.76 | 5.51 | 1.64 | 2.21 | 2.09 | 2.64 |
| 859832 | 2.57 | 0.97 | 1.88 | 3.53 | 2.79 | 3.19 | 1.81 | 2.64 |
| 42313 | 1.33 | 0.51 | 3.37 | 2.77 | 2.16 | 3.35 | 1.55 | 2.64 |
| 282144 | 30.16 | 11.43 | 1.47 | 2.73 | 2.45 | 5.25 | 1.30 | 2.64 |
| 32887 | 3.04 | 1.16 | 1.78 | 3.54 | 3.59 | 2.10 | 2.17 | 2.64 |
| 137254 | 1.35 | 0.51 | 3.82 | 2.00 | 4.43 | 1.38 | 1.55 | 2.64 |
| 234036 | 4.77 | 1.81 | 1.87 | 3.76 | 2.13 | 3.14 | 2.28 | 2.64 |
| 206785 | 6.05 | 2.30 | 4.91 | 3.70 | 1.64 | 1.42 | 1.50 | 2.63 |
| 244202 | 9.25 | 3.51 | 1.51 | 2.29 | 3.36 | 1.92 | 4.09 | 2.63 |
| 827144 | 2.52 | 0.96 | 1.88 | 3.18 | 2.21 | 1.64 | 4.24 | 2.63 |
| 838504 | 41.97 | 15.95 | 1.67 | 3.37 | 2.82 | 3.53 | 1.77 | 2.63 |
| 376764 | 2.58 | 0.98 | 1.35 | 2.16 | 3.58 | 3.03 | 3.03 | 2.63 |
| 744933 | 2.15 | 0.82 | 0.98 | 1.12 | 0.87 | 8.87 | 1.30 | 2.63 |
| 161456 | 13.35 | 5.08 | 0.92 | 0.15 | 10.09 | 0.13 | 1.83 | 2.63 |
| 564846 | 1.90 | 0.72 | 3.47 | 1.34 | 2.53 | 2.17 | 3.62 | 2.63 |
| 815072 | 3.49 | 1.33 | 5.03 | 2.36 | 1.34 | 2.07 | 2.32 | 2.62 |
| 1599489 | 5.51 | 2.10 | 3.32 | 3.51 | 1.94 | 2.95 | 1.40 | 2.62 |
| 80292 | 2.18 | 0.83 | 2.30 | 3.64 | 1.47 | 3.07 | 2.62 | 2.62 |
| 262865 | 30.48 | 11.64 | 3.50 | 2.06 | 4.94 | 0.99 | 1.61 | 2.62 |
| 590390 | 6.42 | 2.45 | 4.07 | 2.94 | 1.83 | 3.34 | 0.92 | 2.62 |
| 610011 | 34.63 | 13.23 | 3.40 | 2.31 | 2.21 | 1.95 | 3.23 | 2.62 |
| 855487 | 8.56 | 3.27 | 2.21 | 3.52 | 3.16 | 1.26 | 2.94 | 2.62 |
| 503097 | 7.27 | 2.78 | 1.87 | 1.80 | 7.01 | 1.54 | 0.86 | 2.62 |
| 825719 | 0.76 | 0.29 | 3.01 | 3.07 | 3.22 | 2.78 | 0.99 | 2.62 |
| 282720 | 8.31 | 3.18 | 3.44 | 1.29 | 2.89 | 2.24 | 3.20 | 2.61 |
| 767798 | 5.70 | 2.18 | 2.43 | 1.90 | 2.16 | 3.46 | 3.13 | 2.61 |
| 784129 | 6.53 | 2.50 | 1.21 | 2.18 | 5.46 | 2.80 | 1.41 | 2.61 |
| 234331 | 3.16 | 1.21 | 2.32 | 2.24 | 3.25 | 2.18 | 3.04 | 2.61 |
| 738970 | 15.96 | 6.13 | 1.21 | 2.29 | 7.41 | 1.31 | 0.82 | 2.61 |
| 41411 | 112.58 | 43.25 | 3.28 | 2.76 | 1.54 | 3.09 | 2.35 | 2.60 |
| 784264 | 5.67 | 2.18 | 1.81 | 3.29 | 2.15 | 3.61 | 2.13 | 2.60 |
| 813249 | 14.02 | 5.40 | 3.66 | 1.96 | 4.73 | 1.34 | 1.29 | 2.60 |
| 840530 | 4.05 | 1.56 | 3.14 | 3.14 | 1.61 | 3.33 | 1.76 | 2.60 |
| 811757 | 1.59 | 0.61 | 3.15 | 2.92 | 3.96 | 1.62 | 1.32 | 2.59 |
| 502369 | 1.41 | 0.55 | 3.58 | 2.45 | 2.17 | 3.01 | 1.74 | 2.59 |
| 744944 | 1.73 | 0.67 | 3.76 | 1.13 | 2.12 | 2.49 | 3.44 | 2.59 |
| 855634 | 3.16 | 1.22 | 1.55 | 1.65 | 3.18 | 1.13 | 5.42 | 2.59 |
| 72616 | 1.67 | 0.65 | 3.07 | 3.09 | 2.14 | 2.94 | 1.65 | 2.58 |
| 436106 | 4.14 | 1.61 | 2.43 | 2.14 | 3.01 | 3.83 | 1.49 | 2.58 |
| 487373 | 54.67 | 21.22 | 2.52 | 2.05 | 1.52 | 3.12 | 3.67 | 2.58 |
| 814154 | 2.29 | 0.89 | 1.83 | 1.77 | 1.85 | 1.52 | 5.87 | 2.57 |
| 435330 | 10.36 | 4.03 | 1.68 | 2.24 | 2.09 | 5.65 | 1.19 | 2.57 |
| 838568 | 53.19 | 20.71 | 1.53 | 3.21 | 1.33 | 4.15 | 2.62 | 2.57 |
| 137638 | 5.06 | 1.97 | 3.16 | 2.54 | 1.89 | 2.17 | 3.08 | 2.57 |
| 41207 | 2.05 | 0.80 | 1.72 | 2.29 | 3.16 | 3.58 | 2.07 | 2.57 |
| 1558642 | 3.11 | 1.21 | 3.27 | 2.26 | 1.72 | 4.08 | 1.50 | 2.56 |
| 432194 | 10.34 | 4.04 | 1.58 | 3.07 | 2.91 | 1.91 | 3.34 | 2.56 |
| 232628 | 4.02 | 1.58 | 4.00 | 1.88 | 4.50 | 1.05 | 1.32 | 2.55 |
| 951022 | 3.02 | 1.18 | 3.66 | 2.02 | 3.80 | 1.61 | 1.67 | 2.55 |
| 809824 | 2.66 | 1.05 | 1.68 | 2.11 | 2.41 | 3.25 | 3.24 | 2.54 |
| 897906 | 10.34 | 4.08 | 1.96 | 2.02 | 4.28 | 0.64 | 3.78 | 2.54 |
| 809722 | 50.59 | 19.96 | 0.71 | 0.97 | 3.43 | 3.59 | 3.97 | 2.54 |
| 138865 | 5.42 | 2.14 | 1.16 | 3.20 | 2.53 | 3.12 | 2.65 | 2.53 |
| 825012 | 4.46 | 1.76 | 3.39 | 2.40 | 2.05 | 3.22 | 1.61 | 2.53 |
| 1472689 | 5.15 | 2.03 | 1.97 | 0.59 | 1.72 | 7.13 | 1.27 | 2.53 |
| 322537 | 3.18 | 1.26 | 1.26 | 6.07 | 2.04 | 0.89 | 2.40 | 2.53 |
| 291974 | 57.24 | 22.63 | 3.29 | 1.87 | 1.09 | 4.48 | 1.91 | 2.53 |
| 307231 | 2.04 | 0.81 | 1.95 | 1.61 | 1.41 | 6.48 | 1.19 | 2.53 |
| 1638479 | 3.27 | 1.30 | 2.08 | 3.88 | 3.23 | 1.78 | 1.67 | 2.53 |
| 415415 | 9.68 | 3.83 | 3.25 | 2.43 | 1.49 | 3.21 | 2.26 | 2.53 |
| 251417 | 0.69 | 0.27 | 3.59 | 3.13 | 1.67 | 1.79 | 2.46 | 2.52 |
| 452936 | 5.15 | 2.04 | 3.47 | 2.84 | 3.54 | 1.65 | 1.11 | 2.52 |
| 488301 | 0.97 | 0.38 | 2.10 | 1.37 | 1.81 | 5.57 | 1.76 | 2.52 |
| 430235 | 3.43 | 1.36 | 4.46 | 3.50 | 1.54 | 1.77 | 1.34 | 2.52 |
| 784085 | 3.93 | 1.56 | 3.14 | 2.04 | 3.39 | 2.55 | 1.49 | 2.52 |
| 741988 | 1.74 | 0.69 | 1.90 | 3.05 | 2.20 | 3.46 | 1.99 | 2.52 |
| 627688 | 7.37 | 2.93 | 1.52 | 1.61 | 6.90 | 1.77 | 0.79 | 2.52 |
| 825859 | 1.64 | 0.65 | 1.30 | 2.26 | 2.23 | 3.48 | 3.31 | 2.52 |
| 884892 | 8.02 | 3.19 | 1.99 | 2.90 | 3.01 | 3.04 | 1.63 | 2.52 |
| 277660 | 14.16 | 5.65 | 1.86 | 2.15 | 2.18 | 3.13 | 3.22 | 2.51 |
| 76605 | 2.48 | 0.99 | 4.39 | 3.21 | 1.27 | 2.11 | 1.53 | 2.50 |
| 220069 | 4.81 | 1.92 | 1.69 | 3.29 | 3.04 | 1.86 | 2.61 | 2.50 |
| 460798 | 1.30 | 0.52 | 1.95 | 1.79 | 3.77 | 1.74 | 3.24 | 2.50 |
| 247546 | 11.16 | 4.48 | 1.50 | 1.33 | 1.37 | 2.54 | 5.72 | 2.49 |
| 814080 | 8.19 | 3.29 | 1.76 | 1.45 | 2.40 | 1.52 | 5.32 | 2.49 |
| 1422794 | 2.62 | 1.05 | 0.95 | 1.99 | 3.82 | 3.31 | 2.38 | 2.49 |
| 51981 | 19.62 | 7.89 | 3.06 | 1.82 | 1.65 | 2.42 | 3.50 | 2.49 |
| 325606 | 7.30 | 2.93 | 3.10 | 2.79 | 1.65 | 3.28 | 1.62 | 2.49 |
| 277596 | 2.91 | 1.17 | 1.17 | 2.54 | 1.50 | 6.20 | 1.01 | 2.49 |
| 788205 | 13.21 | 5.32 | 3.46 | 2.10 | 1.43 | 1.78 | 3.65 | 2.48 |
| 796527 | 1.35 | 0.55 | 2.22 | 1.81 | 1.52 | 5.45 | 1.39 | 2.48 |
| 827132 | 6.63 | 2.68 | 2.47 | 1.83 | 1.62 | 3.44 | 3.03 | 2.48 |
| 842863 | 11.11 | 4.49 | 3.25 | 2.37 | 1.47 | 2.24 | 3.06 | 2.48 |
| 29964 | 1.12 | 0.45 | 1.27 | 1.88 | 1.58 | 4.63 | 3.01 | 2.47 |
| 137984 | 1.63 | 0.66 | 1.72 | 2.25 | 1.34 | 6.05 | 1.01 | 2.47 |
| 767346 | 4.50 | 1.82 | 1.80 | 1.32 | 6.76 | 1.74 | 0.73 | 2.47 |
| 1492967 | 3.68 | 1.49 | 2.19 | 1.49 | 6.63 | 1.16 | 0.89 | 2.47 |
| 342721 | 14.93 | 6.06 | 3.30 | 1.93 | 5.09 | 0.69 | 1.30 | 2.46 |
| 76005 | 2.35 | 0.96 | 4.18 | 3.61 | 1.37 | 1.59 | 1.56 | 2.46 |
| 823850 | 0.78 | 0.32 | 2.69 | 1.51 | 1.29 | 5.06 | 1.76 | 2.46 |
| 744905 | 2.18 | 0.89 | 3.34 | 2.72 | 1.92 | 3.17 | 1.16 | 2.46 |
| 399532 | 6.74 | 2.74 | 1.68 | 2.54 | 1.91 | 3.15 | 3.01 | 2.46 |
| 950464 | 9.75 | 3.98 | 1.50 | 1.01 | 7.67 | 1.11 | 0.97 | 2.45 |
| 486340 | 16.47 | 6.73 | 1.85 | 3.58 | 1.43 | 3.51 | 1.87 | 2.45 |
| 866866 | 3.00 | 1.23 | 4.47 | 3.47 | 1.46 | 1.44 | 1.40 | 2.45 |
| 884546 | 156.92 | 64.25 | 2.76 | 1.70 | 1.46 | 3.06 | 3.23 | 2.44 |
| 897448 | 31.17 | 12.76 | 3.09 | 2.67 | 1.77 | 3.01 | 1.67 | 2.44 |
| 950989 | 2.48 | 1.02 | 4.36 | 3.15 | 1.27 | 1.86 | 1.55 | 2.44 |
| 1031583 | 38.01 | 15.59 | 0.37 | 1.52 | 1.75 | 6.11 | 2.45 | 2.44 |
| 381036 | 3.54 | 1.46 | 2.61 | 1.83 | 5.63 | 1.30 | 0.81 | 2.43 |
| 109316 | 12.35 | 5.08 | 1.34 | 0.40 | 2.84 | 0.34 | 7.26 | 2.43 |
| 448117 | 3.91 | 1.61 | 3.94 | 1.97 | 3.08 | 1.50 | 1.68 | 2.43 |
| 156045 | 2.01 | 0.83 | 3.16 | 3.14 | 1.81 | 2.33 | 1.71 | 2.43 |
| 884333 | 4.10 | 1.69 | 2.33 | 3.28 | 3.72 | 1.30 | 1.51 | 2.43 |
| 306771 | 3.61 | 1.49 | 3.02 | 3.83 | 1.70 | 1.84 | 1.73 | 2.42 |
| 430465 | 5.55 | 2.30 | 0.96 | 0.93 | 1.81 | 7.45 | 0.92 | 2.42 |
| 123755 | 5.06 | 2.10 | 3.75 | 1.13 | 1.03 | 1.10 | 5.05 | 2.41 |
| 42803 | 2.96 | 1.23 | 0.60 | 2.20 | 1.73 | 5.20 | 2.32 | 2.41 |
| 460164 | 0.80 | 0.33 | 2.54 | 3.13 | 2.35 | 3.08 | 0.95 | 2.41 |
| 1574594 | 3.08 | 1.28 | 4.44 | 3.10 | 1.44 | 1.96 | 1.07 | 2.40 |
| 141361 | 1.28 | 0.54 | 1.80 | 0.83 | 1.31 | 6.11 | 1.94 | 2.40 |
| 855336 | 5.69 | 2.37 | 1.65 | 3.16 | 2.36 | 3.39 | 1.43 | 2.40 |
| 868396 | 6.77 | 2.83 | 5.03 | 3.73 | 0.87 | 1.05 | 1.28 | 2.39 |
| 627053 | 11.04 | 4.64 | 1.29 | 2.07 | 5.16 | 2.01 | 1.37 | 2.38 |
| 815772 | 1.44 | 0.61 | 3.35 | 1.86 | 3.10 | 1.62 | 1.96 | 2.38 |
| 897547 | 15.69 | 6.62 | 3.93 | 3.84 | 2.46 | 0.78 | 0.84 | 2.37 |
| 202154 | 29.46 | 12.46 | 0.97 | 2.61 | 0.99 | 5.43 | 1.83 | 2.37 |
| 511107 | 7.14 | 3.02 | 0.73 | 1.70 | 3.61 | 3.54 | 2.25 | 2.36 |
| 810408 | 9.85 | 4.18 | 1.91 | 1.77 | 1.69 | 3.08 | 3.34 | 2.36 |
| 757222 | 5.14 | 2.18 | 2.47 | 6.61 | 1.12 | 0.68 | 0.91 | 2.36 |
| 810734 | 2.46 | 1.05 | 3.06 | 1.80 | 1.50 | 4.04 | 1.32 | 2.35 |
| 1493390 | 3.57 | 1.53 | 3.30 | 1.07 | 2.47 | 3.35 | 1.54 | 2.34 |
| 284865 | 2.87 | 1.23 | 3.64 | 3.21 | 1.66 | 1.49 | 1.70 | 2.34 |
| 488033 | 3.14 | 1.34 | 2.41 | 3.80 | 1.46 | 3.21 | 0.82 | 2.34 |
| 37665 | 1.01 | 0.43 | 0.55 | 3.88 | 2.77 | 3.16 | 1.33 | 2.33 |
| 138861 | 1.72 | 0.74 | 1.34 | 3.12 | 1.63 | 3.22 | 2.35 | 2.33 |
| 272049 | 6.87 | 2.95 | 3.26 | 1.35 | 1.69 | 4.65 | 0.71 | 2.33 |
| 427812 | 9.94 | 4.27 | 2.58 | 1.48 | 5.45 | 1.15 | 0.97 | 2.33 |
| 809995 | 2.80 | 1.21 | 3.69 | 1.55 | 2.04 | 3.10 | 1.24 | 2.32 |
| 449275 | 3.81 | 1.64 | 5.51 | 1.68 | 1.24 | 2.01 | 1.16 | 2.32 |
| 731337 | 0.75 | 0.32 | 3.30 | 3.09 | 1.35 | 1.91 | 1.95 | 2.32 |
| 854570 | 4.11 | 1.78 | 4.79 | 3.45 | 0.94 | 1.54 | 0.83 | 2.31 |
| 767988 | 3.64 | 1.58 | 1.57 | 3.20 | 3.21 | 1.89 | 1.63 | 2.30 |
| 455271 | 1.64 | 0.72 | 5.02 | 2.60 | 1.17 | 1.48 | 1.22 | 2.30 |
| 130136 | 0.29 | 0.13 | 1.17 | 1.08 | 0.84 | 0.80 | 7.57 | 2.29 |
| 757191 | 6.08 | 2.66 | 1.09 | 0.88 | 5.46 | 2.03 | 1.99 | 2.29 |
| 266500 | 7.14 | 3.12 | 1.54 | 3.23 | 1.95 | 3.65 | 1.08 | 2.29 |
| 725503 | 4.55 | 1.99 | 3.35 | 2.31 | 1.33 | 3.39 | 1.03 | 2.28 |
| 1555523 | 1.21 | 0.53 | 1.80 | 6.52 | 1.30 | 0.93 | 0.86 | 2.28 |
| 629498 | 3.73 | 1.64 | 4.45 | 3.42 | 1.09 | 1.19 | 1.23 | 2.28 |
| 781401 | 2.67 | 1.18 | 1.40 | 1.34 | 2.90 | 0.55 | 5.16 | 2.27 |
| 632137 | 0.80 | 0.35 | 4.14 | 1.33 | 3.68 | 1.07 | 1.12 | 2.27 |
| 293830 | 4.43 | 1.96 | 1.87 | 1.37 | 5.66 | 1.25 | 1.18 | 2.26 |
| 50562 | 34.20 | 15.15 | 6.49 | 1.87 | 0.28 | 0.43 | 2.21 | 2.26 |
| 243784 | 2.99 | 1.33 | 3.68 | 3.50 | 1.79 | 1.35 | 0.94 | 2.25 |
| 209167 | 3.29 | 1.46 | 5.12 | 1.17 | 2.88 | 0.92 | 1.17 | 2.25 |
| 251727 | 4.88 | 2.17 | 1.20 | 3.39 | 1.73 | 3.26 | 1.65 | 2.25 |
| 284545 | 1.10 | 0.49 | 1.06 | 3.27 | 0.97 | 4.66 | 1.26 | 2.24 |
| 504290 | 50.88 | 22.72 | 1.27 | 1.74 | 5.93 | 1.07 | 1.18 | 2.24 |
| 71672 | 4.77 | 2.14 | 3.51 | 1.66 | 3.03 | 1.97 | 0.99 | 2.23 |
| 279394 | 3.18 | 1.42 | 1.37 | 1.39 | 5.66 | 0.95 | 1.78 | 2.23 |
| 309864 | 2.49 | 1.12 | 8.06 | 0.69 | 0.90 | 0.79 | 0.67 | 2.22 |
| 291394 | 6.46 | 2.92 | 1.70 | 1.25 | 5.58 | 1.18 | 1.37 | 2.22 |
| 289645 | 0.92 | 0.42 | 1.62 | 1.02 | 1.30 | 5.62 | 1.50 | 2.21 |
| 460218 | 2.10 | 0.95 | 1.71 | 1.76 | 3.37 | 3.35 | 0.86 | 2.21 |
| 856174 | 0.25 | 0.11 | 3.70 | 4.65 | 0.88 | 0.91 | 0.92 | 2.21 |
| 767784 | 8.86 | 4.05 | 5.50 | 1.03 | 1.73 | 1.72 | 0.97 | 2.19 |
| 826459 | 12.36 | 5.65 | 0.94 | 3.16 | 1.94 | 3.18 | 1.72 | 2.19 |
| 141336 | 2.12 | 0.97 | 0.78 | 0.91 | 7.64 | 0.64 | 0.94 | 2.18 |
| 122239 | 6.50 | 2.98 | 4.86 | 3.17 | 0.88 | 0.92 | 1.06 | 2.18 |
| 840698 | 7.45 | 3.42 | 3.42 | 3.27 | 1.39 | 1.58 | 1.22 | 2.18 |
| 303139 | 2.17 | 1.00 | 0.98 | 0.85 | 2.90 | 5.31 | 0.82 | 2.17 |
| 277761 | 2.43 | 1.12 | 3.26 | 1.81 | 1.46 | 3.22 | 1.08 | 2.17 |
| 471641 | 4.79 | 2.22 | 0.68 | 4.45 | 0.89 | 4.15 | 0.64 | 2.16 |
| 470114 | 3.09 | 1.44 | 2.04 | 3.03 | 1.58 | 3.16 | 0.97 | 2.15 |
| 33045 | 0.63 | 0.29 | 5.38 | 1.08 | 1.25 | 1.95 | 1.09 | 2.15 |
| 84078 | 0.34 | 0.16 | 0.79 | 0.75 | 3.03 | 4.97 | 1.17 | 2.14 |
| 111389 | 2.59 | 1.21 | 3.22 | 1.26 | 1.20 | 3.23 | 1.78 | 2.14 |
| 1536991 | 10.79 | 5.05 | 3.61 | 1.25 | 3.84 | 1.06 | 0.92 | 2.14 |
| 770935 | 86.21 | 40.44 | 0.25 | 2.50 | 1.55 | 3.29 | 3.08 | 2.13 |
| 281191 | 7.36 | 3.45 | 3.30 | 1.86 | 3.23 | 0.81 | 1.46 | 2.13 |
| 149895 | 6.05 | 2.84 | 1.33 | 1.44 | 6.25 | 0.94 | 0.69 | 2.13 |
| 648046 | 15.56 | 7.32 | 0.56 | 0.94 | 3.27 | 2.53 | 3.34 | 2.13 |
| 855236 | 2.75 | 1.30 | 1.11 | 1.65 | 1.52 | 5.25 | 1.06 | 2.12 |
| 740780 | 4.71 | 2.25 | 1.78 | 6.08 | 0.90 | 0.38 | 1.35 | 2.10 |
| 844768 | 6.47 | 3.08 | 4.79 | 3.24 | 0.75 | 0.87 | 0.85 | 2.10 |
| 811097 | 2.59 | 1.23 | 5.78 | 1.43 | 0.76 | 1.89 | 0.62 | 2.10 |
| 731255 | 2.04 | 0.98 | 1.12 | 1.83 | 1.35 | 5.14 | 1.01 | 2.09 |
| 950607 | 19.79 | 9.50 | 2.04 | 1.14 | 5.08 | 1.05 | 1.11 | 2.08 |
| 1472150 | 35.19 | 16.93 | 3.00 | 1.41 | 1.13 | 3.73 | 1.12 | 2.08 |
| 52338 | 2.91 | 1.41 | 3.16 | 1.49 | 4.05 | 0.57 | 1.07 | 2.07 |
| 781061 | 0.73 | 0.35 | 0.76 | 1.88 | 3.32 | 3.43 | 0.88 | 2.05 |
| 1613940 | 3.48 | 1.70 | 3.02 | 3.14 | 0.88 | 2.54 | 0.68 | 2.05 |
| 1604717 | 13.38 | 6.59 | 1.03 | 1.41 | 5.95 | 0.92 | 0.85 | 2.03 |
| 151477 | 2.44 | 1.21 | 0.96 | 1.86 | 0.96 | 5.73 | 0.57 | 2.02 |
| 563444 | 6.23 | 3.10 | 1.64 | 3.21 | 0.69 | 3.64 | 0.86 | 2.01 |
| 827168 | 6.51 | 3.24 | 1.91 | 0.82 | 5.31 | 1.07 | 0.95 | 2.01 |
| 845780 | 5.95 | 2.97 | 4.42 | 3.23 | 0.81 | 0.67 | 0.89 | 2.00 |
| 743574 | 6.42 | 3.21 | 4.33 | 3.22 | 0.89 | 0.69 | 0.88 | 2.00 |
| 431597 | 5.45 | 2.73 | 1.67 | 0.89 | 5.75 | 0.72 | 0.97 | 2.00 |
| 740941 | 12.33 | 6.23 | 6.11 | 1.20 | 0.62 | 0.67 | 1.29 | 1.98 |
| 343688 | 6.94 | 3.54 | 1.39 | 1.27 | 5.47 | 0.79 | 0.88 | 1.96 |
| 75059 | 21.82 | 11.17 | 3.00 | 1.01 | 1.04 | 3.21 | 1.50 | 1.95 |
| 782513 | 5.78 | 2.98 | 1.16 | 0.84 | 5.53 | 1.41 | 0.75 | 1.94 |
| 366815 | 71.61 | 36.97 | 5.26 | 1.35 | 1.25 | 0.46 | 1.38 | 1.94 |
| 132911 | 52.56 | 27.26 | 3.07 | 1.21 | 3.40 | 0.74 | 1.22 | 1.93 |
| 282895 | 3.91 | 2.03 | 1.03 | 1.04 | 5.87 | 0.90 | 0.81 | 1.93 |
| 512116 | 4.59 | 2.41 | 1.08 | 0.94 | 5.62 | 1.08 | 0.80 | 1.90 |
| 291706 | 2.41 | 1.27 | 1.34 | 1.06 | 0.98 | 0.91 | 5.20 | 1.90 |
| 50114 | 0.83 | 0.44 | 0.97 | 1.06 | 0.39 | 3.57 | 3.45 | 1.89 |
| 450464 | 3.97 | 2.13 | 3.23 | 1.41 | 3.18 | 0.56 | 0.95 | 1.86 |
| 626462 | 5.86 | 3.24 | 3.91 | 3.03 | 0.70 | 0.59 | 0.81 | 1.81 |
| 867717 | 5.61 | 3.15 | 3.27 | 3.01 | 0.87 | 0.68 | 1.08 | 1.78 |
| 462953 | 3.21 | 1.90 | 0.67 | 5.53 | 1.05 | 0.63 | 0.57 | 1.69 |
| 41287 | 0.72 | 0.45 | 0.73 | 0.95 | 0.53 | 5.14 | 0.61 | 1.59 |
| 122428 | 2.03 | 1.29 | 5.48 | 0.55 | 0.63 | 0.63 | 0.58 | 1.57 |

TABLE 1-2

| | Aver of | Aver. of | MPM-115/ | MPM-116/ | MPM-117/ | MPM-118/ | MPM-119/ | Aver. |
|---|---|---|---|---|---|---|---|---|
| Clone | Tumors | Normals | Aver | Aver | Aver | Aver | Aver Normal | Fold |
| 788180 | 25.18 | 0.81 | 62.98 | 3.35 | 9.48 | 55.67 | 24.72 | 31.24 |
| 646037 | 24.42 | 1.02 | 38.80 | 10.71 | 20.96 | 13.10 | 35.99 | 23.91 |
| 1034473 | 33.61 | 1.51 | 39.30 | 2.42 | 16.40 | 18.87 | 34.35 | 22.27 |
| 281003 | 128.77 | 6.30 | 25.82 | 30.44 | 5.57 | 24.33 | 15.96 | 20.43 |
| 773479 | 12.32 | 0.65 | 24.85 | 24.44 | 9.41 | 16.91 | 18.67 | 18.85 |
| 823940 | 22.39 | 1.32 | 15.54 | 10.31 | 45.60 | 5.40 | 8.09 | 16.99 |
| 281039 | 14.05 | 0.94 | 1.73 | 6.90 | 1.46 | 63.60 | 1.27 | 14.99 |
| 46166 | 13.13 | 0.93 | 2.81 | 31.75 | 16.71 | 15.27 | 4.05 | 14.12 |
| 42415 | 6.86 | 0.51 | 4.67 | 6.71 | 15.78 | 1.60 | 38.87 | 13.52 |
| 291255 | 15.20 | 1.35 | 10.61 | 10.39 | 17.35 | 9.54 | 8.56 | 11.29 |
| 308682 | 8.99 | 0.87 | 8.35 | 12.44 | 6.96 | 9.39 | 14.72 | 10.37 |
| 450049 | 18.37 | 1.83 | 8.45 | 6.05 | 7.47 | 5.21 | 23.07 | 10.05 |
| 283312 | 63.94 | 6.63 | 11.46 | 5.48 | 5.23 | 11.97 | 14.09 | 9.65 |
| 432194 | 10.34 | 1.11 | 5.77 | 11.20 | 10.61 | 6.96 | 12.20 | 9.35 |
| 35626 | 9.42 | 1.09 | 6.10 | 9.04 | 16.67 | 3.16 | 8.40 | 8.67 |
| 222157 | 8.80 | 1.02 | 13.68 | 8.34 | 7.21 | 3.07 | 11.05 | 8.67 |
| 814053 | 14.66 | 1.70 | 8.20 | 1.30 | 6.73 | 19.59 | 7.22 | 8.61 |
| 811867 | 24.57 | 2.97 | 9.33 | 7.93 | 6.31 | 11.68 | 6.18 | 8.28 |
| 35058 | 15.59 | 1.89 | 3.32 | 10.65 | 5.05 | 16.86 | 5.29 | 8.24 |
| 971279 | 54.34 | 6.63 | 8.84 | 8.00 | 7.58 | 8.74 | 7.81 | 8.20 |
| 293635 | 26.47 | 3.44 | 5.13 | 10.21 | 7.80 | 7.55 | 7.78 | 7.69 |
| 32160 | 26.80 | 3.59 | 6.47 | 7.13 | 8.59 | 6.37 | 8.77 | 7.47 |
| 754280 | 23.83 | 3.20 | 15.06 | 7.80 | 8.20 | 3.02 | 3.20 | 7.46 |
| 282089 | 11.18 | 1.51 | 1.86 | 11.16 | 1.40 | 21.82 | 0.82 | 7.41 |
| 745347 | 2.15 | 0.29 | 3.74 | 8.94 | 8.54 | 8.80 | 6.91 | 7.39 |
| 67401 | 1.47 | 0.20 | 1.25 | 1.47 | 30.35 | 2.21 | 1.11 | 7.28 |
| 293651 | 13.35 | 1.84 | 3.60 | 7.75 | 11.33 | 6.53 | 7.12 | 7.26 |
| 784168 | 53.04 | 7.37 | 2.50 | 6.64 | 8.80 | 8.22 | 9.84 | 7.20 |
| 811603 | 19.62 | 2.74 | 8.80 | 6.79 | 5.12 | 7.74 | 7.36 | 7.16 |
| 486035 | 24.75 | 3.47 | 5.79 | 11.37 | 4.91 | 5.88 | 7.72 | 7.13 |
| 809603 | 49.03 | 6.94 | 5.63 | 5.54 | 6.31 | 7.30 | 10.54 | 7.06 |
| 1048810 | 20.15 | 3.04 | 7.11 | 10.78 | 4.14 | 7.28 | 3.86 | 6.63 |
| 756600 | 47.06 | 7.13 | 6.98 | 3.61 | 3.71 | 8.73 | 9.97 | 6.60 |
| 772304 | 113.14 | 18.10 | 7.98 | 4.62 | 4.60 | 5.98 | 8.08 | 6.25 |
| 1631713 | 5.68 | 0.93 | 5.38 | 6.95 | 7.45 | 5.29 | 5.61 | 6.13 |
| 838366 | 11.15 | 1.82 | 4.41 | 5.92 | 6.20 | 5.88 | 8.19 | 6.12 |
| 757435 | 365.78 | 59.86 | 3.80 | 3.09 | 17.09 | 3.11 | 3.46 | 6.11 |
| 1556526 | 0.88 | 0.15 | 0.88 | 2.02 | 1.15 | 24.74 | 1.00 | 5.96 |
| 450307 | 9.39 | 1.62 | 10.72 | 8.38 | 5.22 | 1.77 | 2.92 | 5.80 |
| 796161 | 30.31 | 5.27 | 7.41 | 5.98 | 4.96 | 5.01 | 5.40 | 5.75 |
| 1034738 | 22.40 | 3.90 | 4.98 | 4.85 | 5.57 | 8.17 | 5.15 | 5.74 |
| 49302 | 4.88 | 0.86 | 4.58 | 5.05 | 13.97 | 1.95 | 2.84 | 5.68 |
| 292515 | 16.22 | 2.86 | 4.63 | 8.96 | 3.74 | 4.17 | 6.83 | 5.67 |
| 810220 | 2.17 | 0.39 | 8.61 | 5.96 | 2.67 | 6.95 | 3.82 | 5.60 |
| 207029 | 81.88 | 14.79 | 3.96 | 4.34 | 5.68 | 5.71 | 8.00 | 5.54 |
| 415700 | 3.36 | 0.61 | 4.32 | 5.47 | 9.05 | 2.89 | 5.94 | 5.53 |
| 449112 | 42.54 | 7.72 | 5.75 | 5.33 | 5.65 | 5.97 | 4.85 | 5.51 |
| 531028 | 21.14 | 3.89 | 6.16 | 6.20 | 5.18 | 4.09 | 5.54 | 5.44 |
| 362755 | 16.82 | 3.11 | 6.23 | 6.62 | 4.84 | 4.44 | 4.90 | 5.41 |
| 509887 | 39.47 | 7.39 | 5.15 | 5.67 | 3.89 | 4.14 | 7.85 | 5.34 |
| 191904 | 7.67 | 1.44 | 5.95 | 4.87 | 5.16 | 4.34 | 6.27 | 5.32 |
| 877835 | 23.06 | 4.34 | 7.48 | 3.06 | 4.32 | 5.71 | 6.02 | 5.32 |
| 771317 | 9.44 | 1.78 | 4.49 | 3.90 | 5.78 | 4.69 | 7.70 | 5.31 |
| 549101 | 12.81 | 2.43 | 7.81 | 3.40 | 3.06 | 5.03 | 7.02 | 5.26 |
| 84955 | 5.26 | 1.01 | 8.05 | 6.43 | 6.35 | 2.52 | 2.71 | 5.21 |
| 461670 | 8.90 | 1.72 | 3.21 | 6.57 | 7.68 | 3.75 | 4.73 | 5.19 |
| 197525 | 9.52 | 1.85 | 2.67 | 8.08 | 4.07 | 6.57 | 4.40 | 5.16 |
| 730739 | 36.49 | 7.10 | 4.49 | 4.01 | 10.50 | 3.62 | 3.06 | 5.14 |
| 240694 | 4.53 | 0.90 | 4.65 | 7.82 | 3.10 | 3.41 | 6.08 | 5.01 |
| 593185 | 28.06 | 5.61 | 1.33 | 3.36 | 3.94 | 10.92 | 5.45 | 5.00 |
| 212165 | 41.96 | 8.42 | 7.66 | 4.81 | 6.54 | 2.71 | 3.22 | 4.99 |
| 268211 | 3.43 | 0.70 | 4.14 | 3.18 | 3.13 | 2.23 | 11.97 | 4.93 |
| 233419 | 5.55 | 1.14 | 3.13 | 6.59 | 6.63 | 2.94 | 5.09 | 4.87 |
| 296788 | 3.27 | 0.67 | 4.84 | 4.90 | 7.08 | 2.63 | 4.79 | 4.85 |
| 810558 | 14.49 | 3.00 | 3.18 | 4.47 | 4.01 | 4.93 | 7.57 | 4.83 |
| 264895 | 7.86 | 1.63 | 3.07 | 6.53 | 4.76 | 3.25 | 6.52 | 4.82 |
| 51773 | 11.48 | 2.40 | 5.99 | 3.55 | 3.22 | 7.62 | 3.50 | 4.77 |
| 191603 | 22.14 | 4.64 | 6.96 | 6.89 | 2.33 | 3.85 | 3.81 | 4.77 |
| 417908 | 12.10 | 2.57 | 9.22 | 5.41 | 4.37 | 1.99 | 2.57 | 4.71 |
| 50188 | 10.75 | 2.29 | 5.42 | 4.91 | 4.42 | 3.76 | 4.92 | 4.69 |
| 284701 | 33.05 | 7.05 | 3.72 | 4.93 | 2.53 | 9.40 | 2.86 | 4.68 |
| 416436 | 11.08 | 2.37 | 7.73 | 5.37 | 3.95 | 4.54 | 1.77 | 4.67 |
| 321470 | 7.74 | 1.68 | 4.78 | 6.01 | 4.56 | 2.65 | 5.05 | 4.61 |
| 449275 | 3.81 | 0.83 | 10.88 | 3.32 | 2.44 | 3.97 | 2.28 | 4.58 |
| 263697 | 4.23 | 0.93 | 5.22 | 5.51 | 7.03 | 1.91 | 3.18 | 4.57 |
| 266106 | 20.68 | 4.53 | 4.04 | 4.70 | 9.22 | 2.05 | 2.82 | 4.57 |
| 194364 | 5.44 | 1.19 | 2.81 | 3.35 | 9.83 | 4.15 | 2.64 | 4.56 |
| 259374 | 10.51 | 2.33 | 4.05 | 2.96 | 1.50 | 8.52 | 5.48 | 4.50 |
| 877641 | 67.93 | 15.10 | 8.40 | 2.61 | 1.99 | 5.49 | 3.99 | 4.50 |
| 283173 | 17.85 | 3.97 | 10.94 | 2.48 | 2.73 | 4.55 | 1.75 | 4.49 |
| 868282 | 8.99 | 2.01 | 4.17 | 4.41 | 5.09 | 4.45 | 4.28 | 4.48 |
| 770066 | 11.43 | 2.60 | 1.11 | 1.71 | 4.03 | 13.55 | 1.61 | 4.40 |
| 950688 | 52.65 | 11.99 | 5.18 | 2.58 | 3.06 | 4.94 | 6.20 | 4.39 |
| 490178 | 24.55 | 5.63 | 2.55 | 2.07 | 13.03 | 1.58 | 2.57 | 4.36 |
| 30428 | 4.29 | 0.99 | 0.90 | 2.03 | 2.95 | 11.84 | 4.01 | 4.35 |
| 210405 | 34.02 | 7.84 | 3.39 | 3.97 | 5.43 | 4.21 | 4.69 | 4.34 |
| 795907 | 5.39 | 1.25 | 1.95 | 2.24 | 4.60 | 0.83 | 11.91 | 4.31 |
| 47542 | 8.07 | 1.88 | 6.90 | 3.68 | 2.63 | 4.23 | 4.04 | 4.30 |
| 297439 | 48.60 | 11.33 | 4.18 | 7.63 | 3.83 | 1.95 | 3.86 | 4.29 |
| 1472689 | 5.15 | 1.21 | 3.31 | 0.99 | 2.88 | 11.98 | 2.13 | 4.26 |
| 897767 | 2.07 | 0.49 | 3.67 | 3.81 | 4.12 | 3.26 | 6.41 | 4.25 |
| 289891 | 7.82 | 1.84 | 4.50 | 2.99 | 9.38 | 2.41 | 1.95 | 4.25 |
| 853968 | 12.10 | 2.86 | 5.03 | 5.67 | 7.48 | 1.76 | 1.23 | 4.23 |
| 840788 | 23.03 | 5.46 | 4.18 | 3.11 | 1.97 | 7.77 | 4.07 | 4.22 |
| 856447 | 8.81 | 2.09 | 9.08 | 2.15 | 2.21 | 4.88 | 2.79 | 4.22 |
| 346889 | 0.89 | 0.21 | 2.54 | 5.10 | 1.70 | 6.26 | 5.45 | 4.21 |
| 321706 | 7.80 | 1.86 | 5.63 | 0.98 | 9.88 | 1.19 | 3.34 | 4.20 |
| 855749 | 50.50 | 12.03 | 5.65 | 3.70 | 3.56 | 3.56 | 4.54 | 4.20 |
| 1031583 | 38.01 | 9.06 | 0.63 | 2.62 | 3.01 | 10.51 | 4.21 | 4.20 |
| 785913 | 3.35 | 0.80 | 4.02 | 3.95 | 7.94 | 2.07 | 2.99 | 4.19 |
| 746152 | 9.35 | 2.23 | 7.33 | 4.05 | 7.60 | 1.47 | 0.48 | 4.18 |
| 845363 | 29.59 | 7.07 | 4.62 | 3.53 | 2.38 | 5.97 | 4.42 | 4.18 |
| 377246 | 12.60 | 3.02 | 3.68 | 2.77 | 11.38 | 1.14 | 1.90 | 4.17 |
| 129024 | 4.30 | 1.04 | 7.23 | 0.75 | 2.32 | 2.43 | 7.90 | 4.13 |
| 150262 | 13.66 | 3.31 | 3.87 | 5.14 | 4.58 | 3.46 | 3.56 | 4.12 |
| 825207 | 11.96 | 2.92 | 3.12 | 3.72 | 4.18 | 5.49 | 3.94 | 4.09 |
| 1030855 | 14.86 | 3.64 | 1.14 | 4.82 | 2.58 | 7.86 | 4.02 | 4.08 |
| 878127 | 3.53 | 0.87 | 2.76 | 4.48 | 6.05 | 4.44 | 2.62 | 4.07 |
| 138444 | 4.04 | 1.00 | 3.68 | 7.21 | 2.94 | 4.04 | 2.44 | 4.06 |
| 768395 | 14.29 | 3.54 | 5.36 | 4.80 | 4.35 | 3.14 | 2.53 | 4.03 |
| 788541 | 12.11 | 3.01 | 2.87 | 3.90 | 7.56 | 1.94 | 3.89 | 4.03 |
| 1468260 | 4.53 | 1.13 | 7.56 | 0.78 | 4.31 | 2.33 | 5.15 | 4.03 |
| 787938 | 5.09 | 1.27 | 1.46 | 4.56 | 6.98 | 5.35 | 1.72 | 4.01 |
| 258300 | 18.15 | 4.53 | 2.52 | 4.17 | 7.79 | 0.73 | 4.84 | 4.01 |
| 208082 | 7.90 | 1.98 | 3.29 | 6.85 | 3.52 | 3.30 | 3.00 | 3.99 |
| 785910 | 4.01 | 1.01 | 3.80 | 2.26 | 9.32 | 1.33 | 3.21 | 3.98 |
| 287728 | 2.76 | 0.70 | 0.75 | 5.13 | 1.25 | 11.82 | 0.89 | 3.97 |
| 687297 | 12.18 | 3.12 | 4.57 | 4.32 | 4.27 | 2.73 | 3.63 | 3.90 |
| 741067 | 64.30 | 16.50 | 0.91 | 4.38 | 2.37 | 7.31 | 4.51 | 3.90 |
| 1031598 | 5.18 | 1.34 | 0.69 | 2.70 | 2.63 | 9.79 | 3.58 | 3.88 |
| 366815 | 71.61 | 18.49 | 10.51 | 2.69 | 2.49 | 0.91 | 2.76 | 3.87 |
| 323474 | 1.25 | 0.32 | 6.77 | 4.12 | 2.60 | 2.99 | 2.88 | 3.87 |
| 288695 | 3.41 | 0.88 | 4.88 | 4.64 | 5.25 | 3.06 | 1.48 | 3.86 |
| 204111 | 3.38 | 0.87 | 3.40 | 4.11 | 3.20 | 3.52 | 5.07 | 3.86 |
| 242952 | 16.94 | 4.39 | 4.50 | 3.65 | 3.98 | 4.16 | 2.99 | 3.86 |
| 1631747 | 5.09 | 1.33 | 5.74 | 6.76 | 2.76 | 2.44 | 1.49 | 3.84 |
| 48286 | 2.34 | 0.61 | 3.34 | 2.74 | 4.95 | 3.47 | 4.67 | 3.83 |
| 745503 | 42.62 | 11.14 | 2.97 | 4.02 | 3.39 | 3.08 | 5.68 | 3.83 |
| 744846 | 2.69 | 0.70 | 4.56 | 5.57 | 3.31 | 4.45 | 1.24 | 3.83 |
| 454771 | 2.69 | 0.70 | 7.92 | 2.22 | 2.30 | 1.94 | 4.70 | 3.82 |
| 233464 | 14.24 | 3.73 | 2.79 | 2.89 | 5.83 | 3.29 | 4.26 | 3.81 |
| 250869 | 123.92 | 32.54 | 10.99 | 2.78 | 2.27 | 1.03 | 1.97 | 3.81 |
| 842925 | 4.61 | 1.21 | 3.52 | 3.54 | 4.61 | 4.62 | 2.67 | 3.79 |
| 843094 | 49.18 | 13.00 | 4.51 | 3.53 | 3.71 | 3.00 | 4.17 | 3.78 |
| 587415 | 12.98 | 3.44 | 6.76 | 3.20 | 6.42 | 1.08 | 1.39 | 3.77 |
| 33715 | 1.36 | 0.37 | 2.84 | 3.84 | 4.39 | 3.91 | 3.66 | 3.73 |
| 75044 | 3.46 | 0.93 | 4.59 | 3.85 | 2.61 | 3.71 | 3.84 | 3.72 |
| 1467207 | 3.68 | 0.99 | 2.51 | 4.57 | 4.41 | 2.93 | 4.17 | 3.72 |
| 366132 | 6.22 | 1.68 | 4.94 | 2.88 | 2.87 | 4.12 | 3.77 | 3.72 |
| 813973 | 3.24 | 0.88 | 3.24 | 3.78 | 4.03 | 3.23 | 4.23 | 3.70 |
| 814017 | 86.17 | 23.42 | 5.13 | 5.73 | 3.05 | 1.59 | 2.90 | 3.68 |
| 785616 | 11.47 | 3.12 | 4.63 | 4.47 | 3.42 | 3.47 | 2.37 | 3.67 |
| 42803 | 2.96 | 0.81 | 0.91 | 3.34 | 2.63 | 7.89 | 3.52 | 3.66 |
| 488964 | 7.77 | 2.13 | 11.81 | 2.73 | 1.04 | 1.76 | 0.88 | 3.64 |
| 151055 | 27.32 | 7.51 | 3.65 | 6.52 | 1.80 | 2.91 | 3.31 | 3.64 |
| 784258 | 10.08 | 2.77 | 4.79 | 2.92 | 5.87 | 1.93 | 2.67 | 3.64 |
| 81331 | 2.84 | 0.78 | 1.10 | 2.95 | 0.88 | 12.05 | 1.18 | 3.63 |
| 897042 | 4.95 | 1.36 | 6.51 | 3.85 | 2.87 | 3.80 | 1.14 | 3.63 |
| 771048 | 10.54 | 2.90 | 4.89 | 3.20 | 3.32 | 2.85 | 3.91 | 3.63 |
| 795265 | 4.58 | 1.26 | 1.26 | 2.63 | 2.84 | 9.65 | 1.76 | 3.63 |
| 753252 | 7.78 | 2.14 | 2.88 | 3.17 | 3.38 | 4.62 | 4.10 | 3.63 |
| 430465 | 5.55 | 1.53 | 1.44 | 1.39 | 2.71 | 11.18 | 1.38 | 3.62 |
| 429678 | 3.12 | 0.86 | 3.79 | 2.41 | 5.14 | 4.64 | 2.08 | 3.61 |
| 47384 | 4.22 | 1.17 | 2.95 | 3.72 | 4.97 | 3.42 | 2.98 | 3.61 |
| 342271 | 3.90 | 1.09 | 3.73 | 1.46 | 4.16 | 6.29 | 2.35 | 3.60 |
| 712950 | 11.53 | 3.21 | 0.83 | 1.53 | 3.05 | 11.90 | 0.65 | 3.59 |
| 245547 | 5.86 | 1.63 | 1.33 | 6.79 | 3.91 | 4.02 | 1.91 | 3.59 |
| 1475987 | 2.96 | 0.83 | 4.18 | 2.40 | 3.01 | 6.04 | 2.26 | 3.58 |
| 358850 | 21.29 | 5.98 | 6.12 | 3.37 | 2.69 | 1.80 | 3.82 | 3.56 |
| 45327 | 8.18 | 2.30 | 2.35 | 3.39 | 3.66 | 4.62 | 3.78 | 3.56 |
| 950410 | 3.18 | 0.89 | 1.81 | 7.55 | 3.00 | 2.68 | 2.76 | 3.56 |
| 1592006 | 5.30 | 1.51 | 4.10 | 3.01 | 3.34 | 3.68 | 3.47 | 3.52 |
| 741474 | 4.93 | 1.40 | 2.90 | 2.23 | 3.62 | 5.57 | 3.24 | 3.51 |
| 292042 | 9.39 | 2.67 | 3.33 | 4.22 | 4.13 | 3.14 | 2.73 | 3.51 |
| 280217 | 20.52 | 5.86 | 4.76 | 0.46 | 1.34 | 10.23 | 0.72 | 3.50 |
| 42636 | 1.11 | 0.32 | 2.78 | 3.16 | 4.86 | 2.57 | 4.13 | 3.50 |
| 550141 | 78.20 | 22.40 | 5.62 | 2.32 | 2.57 | 3.16 | 3.79 | 3.49 |
| 159608 | 14.43 | 4.14 | 6.82 | 1.07 | 1.06 | 5.09 | 3.39 | 3.49 |
| 383185 | 12.04 | 3.46 | 6.25 | 2.54 | 6.69 | 0.98 | 0.94 | 3.48 |
| 491184 | 6.35 | 1.83 | 4.07 | 1.43 | 10.01 | 0.85 | 1.00 | 3.47 |
| 786525 | 1.46 | 0.42 | 2.15 | 1.29 | 2.53 | 10.04 | 1.31 | 3.46 |
| 305538 | 11.44 | 3.31 | 4.78 | 4.20 | 1.98 | 1.85 | 4.49 | 3.46 |
| 415437 | 3.52 | 1.02 | 2.69 | 3.45 | 3.10 | 4.16 | 3.88 | 3.46 |
| 259350 | 22.84 | 6.64 | 2.91 | 2.83 | 2.97 | 3.77 | 4.71 | 3.44 |
| 950709 | 4.17 | 1.22 | 5.93 | 2.37 | 2.11 | 4.13 | 2.61 | 3.43 |
| 141505 | 6.38 | 1.86 | 2.88 | 4.20 | 3.06 | 2.08 | 4.90 | 3.42 |
| 109200 | 3.17 | 0.93 | 5.89 | 2.59 | 3.19 | 2.96 | 2.46 | 3.42 |
| 1390584 | 6.54 | 1.91 | 1.98 | 3.12 | 3.26 | 8.11 | 0.63 | 3.42 |
| 823819 | 11.96 | 3.51 | 4.50 | 4.09 | 2.11 | 2.94 | 3.43 | 3.41 |
| 272750 | 20.57 | 6.04 | 3.75 | 5.31 | 2.77 | 2.65 | 2.54 | 3.40 |
| 246869 | 4.68 | 1.38 | 4.93 | 2.40 | 3.61 | 2.07 | 3.98 | 3.40 |
| 784253 | 11.76 | 3.47 | 2.81 | 4.36 | 3.28 | 2.90 | 3.61 | 3.39 |
| 490766 | 3.48 | 1.03 | 4.47 | 3.60 | 2.64 | 2.91 | 3.27 | 3.38 |
| 356863 | 3.18 | 0.94 | 4.17 | 3.83 | 2.02 | 3.24 | 3.63 | 3.38 |
| 837904 | 103.13 | 30.63 | 2.21 | 2.23 | 2.11 | 4.52 | 5.77 | 3.37 |
| 462468 | 3.24 | 0.96 | 3.67 | 4.25 | 3.86 | 1.95 | 3.06 | 3.36 |
| 121997 | 5.10 | 1.52 | 4.66 | 2.43 | 5.54 | 1.60 | 2.55 | 3.36 |
| 700900 | 9.94 | 3.00 | 3.26 | 2.88 | 3.29 | 3.75 | 3.40 | 3.31 |
| 26616 | 1.94 | 0.59 | 4.02 | 2.79 | 3.28 | 3.05 | 3.40 | 3.31 |
| 1589973 | 5.17 | 1.57 | 2.96 | 3.72 | 2.30 | 5.22 | 2.31 | 3.30 |
| 392678 | 34.95 | 10.61 | 2.24 | 2.72 | 3.38 | 3.80 | 4.32 | 3.29 |
| 586725 | 2.32 | 0.70 | 3.69 | 2.64 | 2.18 | 3.31 | 4.64 | 3.29 |
| 121489 | 34.70 | 10.55 | 3.98 | 4.08 | 3.24 | 2.67 | 2.47 | 3.29 |
| 503851 | 18.15 | 5.54 | 1.87 | 3.27 | 5.43 | 2.90 | 2.90 | 3.28 |
| 854874 | 2.22 | 0.68 | 2.50 | 3.02 | 5.99 | 2.47 | 2.32 | 3.26 |
| 877832 | 44.43 | 13.65 | 3.45 | 3.31 | 2.92 | 3.09 | 3.50 | 3.25 |
| 253009 | 115.86 | 35.75 | 1.83 | 3.37 | 1.51 | 5.38 | 4.13 | 3.24 |
| 824723 | 14.11 | 4.37 | 2.37 | 2.55 | 3.40 | 5.30 | 2.52 | 3.23 |
| 755409 | 7.81 | 2.43 | 3.30 | 3.12 | 4.05 | 3.18 | 2.47 | 3.22 |
| 506564 | 12.93 | 4.03 | 4.44 | 2.86 | 3.21 | 1.98 | 3.55 | 3.21 |
| 109153 | 5.46 | 1.70 | 4.21 | 2.85 | 3.08 | 2.33 | 3.55 | 3.20 |
| 213682 | 13.07 | 4.10 | 0.63 | 2.78 | 2.14 | 7.22 | 3.16 | 3.19 |
| 133717 | 9.46 | 2.97 | 2.19 | 2.22 | 3.36 | 2.66 | 5.49 | 3.18 |
| 361659 | 13.43 | 4.23 | 1.54 | 3.83 | 4.60 | 3.93 | 1.99 | 3.18 |
| 232628 | 4.02 | 1.27 | 4.95 | 2.33 | 5.56 | 1.30 | 1.63 | 3.16 |
| 283034 | 9.61 | 3.05 | 3.07 | 5.05 | 2.86 | 2.31 | 2.46 | 3.15 |
| 490232 | 2.68 | 0.85 | 3.64 | 3.48 | 3.06 | 1.82 | 3.75 | 3.15 |
| 781366 | 12.06 | 3.84 | 3.48 | 2.65 | 1.25 | 2.22 | 6.10 | 3.14 |
| 66317 | 5.50 | 1.76 | 6.84 | 2.02 | 1.77 | 4.59 | 0.44 | 3.13 |
| 359781 | 7.82 | 2.50 | 1.84 | 5.41 | 2.09 | 3.84 | 2.45 | 3.13 |
| 564801 | 12.35 | 3.96 | 2.96 | 1.38 | 8.87 | 1.73 | 0.67 | 3.12 |
| 587595 | 559.22 | 179.15 | 0.74 | 2.46 | 3.02 | 6.40 | 2.98 | 3.12 |
| 39885 | 0.94 | 0.30 | 0.92 | 2.92 | 4.86 | 4.97 | 1.87 | 3.11 |
| 45578 | 5.05 | 1.63 | 4.85 | 2.27 | 2.38 | 3.60 | 2.39 | 3.10 |
| 743749 | 5.10 | 1.65 | 4.98 | 3.05 | 2.98 | 3.05 | 1.42 | 3.10 |
| 839081 | 16.60 | 5.37 | 5.25 | 5.49 | 0.97 | 2.62 | 1.11 | 3.09 |
| 109316 | 12.35 | 4.01 | 1.69 | 0.50 | 3.59 | 0.43 | 9.18 | 3.08 |
| 358083 | 2.45 | 0.80 | 3.44 | 2.58 | 4.41 | 1.95 | 2.99 | 3.07 |
| 130773 | 3.71 | 1.21 | 2.86 | 3.98 | 3.19 | 2.42 | 2.91 | 3.07 |
| 610011 | 34.63 | 11.27 | 3.99 | 2.71 | 2.59 | 2.28 | 3.79 | 3.07 |
| 429574 | 3.77 | 1.23 | 2.74 | 4.01 | 3.30 | 1.01 | 4.26 | 3.06 |
| 167205 | 7.85 | 2.56 | 2.90 | 5.74 | 3.48 | 1.88 | 1.31 | 3.06 |
| 825718 | 4.03 | 1.32 | 2.59 | 2.65 | 4.00 | 3.21 | 2.84 | 3.06 |
| 42659 | 10.21 | 3.36 | 3.63 | 3.18 | 3.06 | 2.09 | 3.23 | 3.04 |
| 1631682 | 1.61 | 0.53 | 4.08 | 2.09 | 3.06 | 2.23 | 3.71 | 3.03 |
| 843234 | 7.02 | 2.32 | 3.12 | 3.14 | 2.40 | 2.69 | 3.80 | 3.03 |
| 290642 | 7.76 | 2.56 | 4.18 | 2.71 | 2.32 | 3.00 | 2.91 | 3.02 |
| 51700 | 3.66 | 1.21 | 3.02 | 2.75 | 3.37 | 2.08 | 3.87 | 3.02 |
| 214006 | 4.67 | 1.55 | 6.05 | 4.62 | 1.41 | 2.11 | 0.89 | 3.02 |
| 431286 | 13.81 | 4.59 | 2.40 | 3.23 | 3.52 | 2.10 | 3.80 | 3.01 |
| 781454 | 9.75 | 3.24 | 2.57 | 1.19 | 9.17 | 1.13 | 0.98 | 3.01 |
| 433253 | 7.29 | 2.43 | 2.31 | 2.35 | 2.05 | 5.26 | 3.03 | 3.00 |
| 359661 | 26.24 | 8.79 | 2.02 | 4.95 | 1.82 | 3.95 | 2.19 | 2.99 |
| 811953 | 17.84 | 5.98 | 3.25 | 3.61 | 3.69 | 1.97 | 2.41 | 2.98 |
| 196345 | 122.93 | 41.22 | 1.48 | 2.47 | 2.71 | 3.86 | 4.39 | 2.98 |
| 950473 | 9.36 | 3.14 | 2.36 | 3.35 | 2.97 | 2.65 | 3.56 | 2.98 |
| 46294 | 1.03 | 0.35 | 1.45 | 2.41 | 1.44 | 5.75 | 3.83 | 2.97 |
| 37980 | 0.84 | 0.29 | 1.32 | 2.54 | 1.61 | 7.51 | 1.81 | 2.96 |
| 512116 | 4.59 | 1.55 | 1.67 | 1.46 | 8.72 | 1.68 | 1.24 | 2.96 |
| 137638 | 5.06 | 1.71 | 3.63 | 2.92 | 2.17 | 2.49 | 3.54 | 2.95 |
| 810753 | 4.95 | 1.68 | 3.05 | 2.91 | 3.48 | 2.96 | 2.35 | 2.95 |
| 592403 | 0.46 | 0.16 | 0.70 | 2.31 | 2.27 | 6.90 | 2.56 | 2.95 |
| 273546 | 9.10 | 3.09 | 4.02 | 3.24 | 2.32 | 2.44 | 2.71 | 2.95 |
| 238821 | 6.54 | 2.22 | 3.78 | 1.07 | 3.22 | 0.62 | 6.02 | 2.94 |
| 111812 | 5.56 | 1.89 | 3.45 | 4.26 | 4.44 | 1.20 | 1.35 | 2.94 |
| 840894 | 34.81 | 11.87 | 1.12 | 3.23 | 2.53 | 4.70 | 3.09 | 2.93 |
| 50114 | 0.83 | 0.28 | 1.50 | 1.65 | 0.60 | 5.53 | 5.35 | 2.92 |
| 971367 | 35.22 | 12.06 | 3.23 | 1.99 | 1.70 | 5.64 | 2.05 | 2.92 |
| 1473300 | 12.44 | 4.26 | 1.78 | 2.25 | 2.22 | 3.03 | 5.31 | 2.92 |
| 824704 | 2.04 | 0.70 | 3.07 | 2.49 | 3.23 | 2.63 | 3.15 | 2.91 |
| 897670 | 6.35 | 2.19 | 3.64 | 3.98 | 2.74 | 2.73 | 1.43 | 2.90 |
| 430153 | 4.46 | 1.54 | 3.45 | 3.54 | 2.88 | 1.69 | 2.95 | 2.90 |
| 31225 | 4.76 | 1.64 | 2.16 | 2.29 | 4.85 | 2.20 | 3.01 | 2.90 |
| 743828 | 3.34 | 1.15 | 1.18 | 1.72 | 5.33 | 1.00 | 5.28 | 2.90 |
| 1521977 | 2.08 | 0.72 | 3.62 | 1.94 | 2.94 | 3.07 | 2.92 | 2.90 |
| 511632 | 7.85 | 2.71 | 2.19 | 2.68 | 1.95 | 4.09 | 3.56 | 2.89 |
| 360885 | 40.29 | 13.93 | 4.07 | 2.17 | 4.57 | 1.22 | 2.42 | 2.89 |
| 1492238 | 13.96 | 4.83 | 3.53 | 3.13 | 2.20 | 3.54 | 2.04 | 2.89 |
| 784830 | 1.83 | 0.63 | 4.04 | 1.72 | 3.99 | 1.90 | 2.77 | 2.88 |
| 767798 | 5.70 | 1.98 | 2.68 | 2.09 | 2.38 | 3.81 | 3.45 | 2.88 |
| 840590 | 21.70 | 7.54 | 2.19 | 1.06 | 2.15 | 7.48 | 1.51 | 2.88 |
| 878316 | 5.34 | 1.86 | 2.30 | 2.73 | 1.60 | 4.36 | 3.36 | 2.87 |
| 725489 | 26.71 | 9.34 | 3.59 | 3.28 | 2.57 | 3.29 | 1.57 | 2.86 |
| 209167 | 3.29 | 1.15 | 6.50 | 1.48 | 3.65 | 1.17 | 1.48 | 2.86 |
| 488431 | 10.48 | 3.68 | 4.07 | 2.58 | 2.02 | 1.74 | 3.84 | 2.85 |
| 731308 | 4.84 | 1.70 | 3.77 | 3.06 | 2.31 | 1.95 | 3.15 | 2.85 |
| 565235 | 35.83 | 12.59 | 1.31 | 3.41 | 1.55 | 7.11 | 0.85 | 2.85 |
| 504290 | 50.88 | 17.89 | 1.61 | 2.21 | 7.54 | 1.36 | 1.50 | 2.84 |
| 823588 | 7.72 | 2.72 | 2.42 | 3.62 | 3.20 | 2.49 | 2.44 | 2.83 |
| 878544 | 19.20 | 6.78 | 5.95 | 1.60 | 3.38 | 1.81 | 1.41 | 2.83 |
| 26736 | 9.52 | 3.36 | 3.45 | 0.77 | 0.57 | 6.03 | 3.33 | 2.83 |
| 145388 | 2.66 | 0.94 | 3.12 | 3.26 | 4.21 | 2.28 | 1.26 | 2.83 |
| 76196 | 2.53 | 0.90 | 1.68 | 2.14 | 2.06 | 3.22 | 5.03 | 2.83 |
| 624271 | 5.74 | 2.03 | 3.08 | 2.14 | 1.61 | 6.02 | 1.25 | 2.82 |
| 66902 | 3.21 | 1.14 | 3.84 | 1.85 | 1.41 | 0.79 | 6.22 | 2.82 |
| 86160 | 2.10 | 0.74 | 2.49 | 3.24 | 2.74 | 2.51 | 3.12 | 2.82 |
| 526945 | 1.14 | 0.40 | 3.22 | 1.77 | 2.51 | 3.16 | 3.41 | 2.82 |
| 131621 | 10.21 | 3.63 | 3.64 | 1.77 | 4.66 | 1.90 | 2.11 | 2.82 |
| 815036 | 1.62 | 0.58 | 3.61 | 3.75 | 2.37 | 3.01 | 1.33 | 2.81 |
| 416833 | 6.07 | 2.16 | 1.37 | 3.86 | 1.76 | 4.96 | 2.11 | 2.81 |
| 810221 | 3.85 | 1.37 | 1.41 | 2.35 | 3.96 | 2.88 | 3.43 | 2.81 |
| 1536991 | 10.79 | 3.85 | 4.74 | 1.63 | 5.04 | 1.39 | 1.21 | 2.80 |
| 1240220 | 7.60 | 2.71 | 0.70 | 2.40 | 5.33 | 4.42 | 1.16 | 2.80 |
| 125308 | 2.61 | 0.93 | 4.31 | 2.08 | 3.64 | 2.05 | 1.93 | 2.80 |
| 502690 | 4.09 | 1.46 | 2.11 | 3.13 | 2.39 | 4.17 | 2.19 | 2.80 |
| 1635874 | 6.06 | 2.17 | 5.71 | 2.90 | 1.66 | 2.16 | 1.54 | 2.79 |
| 32076 | 1.03 | 0.37 | 1.94 | 2.97 | 2.60 | 3.25 | 3.21 | 2.79 |
| 856535 | 1.40 | 0.50 | 3.08 | 3.52 | 2.81 | 2.44 | 2.11 | 2.79 |
| 30793 | 7.89 | 2.84 | 2.22 | 2.49 | 3.31 | 3.04 | 2.85 | 2.78 |
| 826102 | 4.19 | 1.51 | 1.94 | 1.78 | 7.59 | 1.08 | 1.50 | 2.78 |
| 356992 | 21.95 | 7.93 | 3.72 | 2.43 | 2.23 | 3.22 | 2.25 | 2.77 |
| 810571 | 4.37 | 1.58 | 1.62 | 3.67 | 1.77 | 4.25 | 2.52 | 2.76 |
| 365098 | 4.93 | 1.78 | 4.88 | 1.79 | 3.50 | 1.53 | 2.10 | 2.76 |
| 788655 | 7.66 | 2.78 | 2.38 | 1.89 | 4.08 | 3.62 | 1.83 | 2.76 |
| 186918 | 3.04 | 1.10 | 1.49 | 2.82 | 2.33 | 5.76 | 1.40 | 2.76 |
| 842784 | 326.54 | 118.59 | 0.68 | 2.49 | 1.56 | 5.61 | 3.44 | 2.75 |
| 127396 | 14.07 | 5.12 | 2.63 | 3.54 | 0.99 | 4.76 | 1.83 | 2.75 |
| 261472 | 9.31 | 3.39 | 2.65 | 1.79 | 2.40 | 5.00 | 1.89 | 2.75 |
| 813179 | 6.60 | 2.40 | 1.14 | 8.50 | 0.98 | 2.38 | 0.73 | 2.75 |
| 240430 | 3.52 | 1.28 | 5.57 | 1.49 | 1.68 | 2.99 | 1.98 | 2.74 |
| 1435029 | 1.84 | 0.67 | 2.78 | 3.83 | 3.22 | 2.31 | 1.55 | 2.74 |
| 124824 | 30.85 | 11.28 | 2.73 | 1.75 | 1.56 | 2.43 | 5.21 | 2.73 |
| 1493390 | 3.57 | 1.31 | 3.84 | 1.25 | 2.88 | 3.90 | 1.79 | 2.73 |
| 251936 | 7.48 | 2.74 | 3.40 | 3.01 | 2.19 | 2.17 | 2.89 | 2.73 |
| 343609 | 1.58 | 0.58 | 2.30 | 2.63 | 3.03 | 2.56 | 3.13 | 2.73 |
| 125722 | 3.28 | 1.21 | 4.28 | 2.15 | 2.60 | 3.74 | 0.81 | 2.72 |
| 322051 | 11.58 | 4.27 | 2.00 | 2.69 | 3.96 | 1.51 | 3.38 | 2.71 |
| 756556 | 17.53 | 6.47 | 1.96 | 2.33 | 5.07 | 2.59 | 1.60 | 2.71 |
| 430318 | 10.87 | 4.02 | 1.74 | 2.76 | 3.55 | 1.94 | 3.54 | 2.71 |
| 795555 | 3.38 | 1.25 | 3.07 | 3.53 | 2.44 | 2.31 | 2.14 | 2.70 |
| 951125 | 18.86 | 7.00 | 2.18 | 5.16 | 1.53 | 2.32 | 2.28 | 2.69 |
| 843150 | 5.13 | 1.90 | 2.80 | 3.47 | 2.10 | 1.72 | 3.38 | 2.69 |
| 50772 | 18.10 | 6.74 | 1.30 | 2.66 | 2.08 | 5.85 | 1.54 | 2.68 |
| 8050014 | 0.81 | 52.48 | 0.85 | 2.49 | 1.54 | 4.92 | 3.61 | 2.68 |
| 137189 | 5.26 | 1.96 | 1.82 | 3.19 | 2.48 | 3.67 | 2.23 | 2.68 |
| 279482 | 8.66 | 3.25 | 2.09 | 1.33 | 7.28 | 0.95 | 1.69 | 2.67 |
| 745103 | 8.87 | 3.33 | 1.82 | 3.08 | 3.04 | 1.84 | 3.55 | 2.67 |
| 760148 | 2.93 | 1.10 | 2.27 | 2.03 | 3.48 | 3.23 | 2.28 | 2.66 |
| 823615 | 9.40 | 3.54 | 3.54 | 1.46 | 5.77 | 1.58 | 0.93 | 2.65 |
| 783645 | 6.96 | 2.63 | 3.48 | 2.53 | 3.22 | 1.85 | 2.18 | 2.65 |
| 81578 | 12.77 | 4.82 | 3.38 | 2.44 | 4.22 | 1.59 | 1.60 | 2.65 |
| 785793 | 55.68 | 21.05 | 3.09 | 2.27 | 3.82 | 1.77 | 2.28 | 2.65 |
| 277660 | 14.16 | 5.37 | 1.96 | 2.26 | 2.29 | 3.29 | 3.39 | 2.64 |
| 66336 | 1.67 | 0.63 | 4.14 | 4.60 | 4.12 | 0.16 | 0.16 | 2.63 |
| 1630990 | 20.64 | 7.84 | 4.29 | 2.18 | 1.71 | 4.51 | 0.48 | 2.63 |
| 247609 | 3.15 | 1.20 | 2.53 | 1.52 | 2.16 | 3.23 | 3.74 | 2.63 |
| 898123 | 4.77 | 1.81 | 1.92 | 3.20 | 3.04 | 1.92 | 3.07 | 2.63 |
| 40136 | 5.23 | 1.99 | 1.90 | 1.77 | 2.01 | 1.67 | 5.77 | 2.62 |
| 46367 | 1.63 | 0.62 | 1.73 | 1.61 | 1.24 | 7.45 | 1.09 | 2.62 |
| 281072 | 5.02 | 1.92 | 1.08 | 2.04 | 1.92 | 5.46 | 2.57 | 2.62 |
| 1606837 | 23.76 | 9.09 | 3.80 | 1.46 | 1.83 | 3.18 | 2.79 | 2.61 |
| 308726 | 14.73 | 5.64 | 3.93 | 2.00 | 4.67 | 0.90 | 1.56 | 2.61 |
| 784116 | 0.26 | 0.10 | 2.07 | 3.32 | 3.40 | 2.11 | 2.15 | 2.61 |
| 46620 | 5.25 | 2.02 | 1.48 | 2.20 | 1.83 | 3.69 | 3.83 | 2.61 |
| 504544 | 12.31 | 4.73 | 1.63 | 2.83 | 1.76 | 5.44 | 1.35 | 2.60 |
| 279394 | 3.18 | 1.23 | 1.59 | 1.61 | 6.58 | 1.11 | 2.07 | 2.59 |
| 262865 | 30.48 | 11.78 | 3.45 | 2.03 | 4.88 | 0.98 | 1.59 | 2.59 |
| 123980 | 2.38 | 0.92 | 1.80 | 3.18 | 2.76 | 1.49 | 3.71 | 2.59 |
| 293830 | 4.43 | 1.72 | 2.14 | 1.56 | 6.45 | 1.42 | 1.34 | 2.58 |
| 383188 | 2.79 | 1.08 | 3.03 | 2.15 | 1.40 | 2.92 | 3.40 | 2.58 |
| 853998 | 12.39 | 4.81 | 1.79 | 2.00 | 3.76 | 2.32 | 3.01 | 2.58 |
| 813651 | 4.96 | 1.93 | 1.72 | 2.40 | 2.48 | 3.04 | 3.24 | 2.58 |
| 203350 | 1.91 | 0.74 | 2.76 | 1.43 | 3.30 | 3.24 | 2.14 | 2.57 |
| 240961 | 2.11 | 0.82 | 2.13 | 4.67 | 3.19 | 1.71 | 1.17 | 2.57 |
| 1035765 | 6.21 | 2.41 | 2.60 | 0.83 | 3.16 | 0.47 | 5.78 | 2.57 |
| 970271 | 3.16 | 1.23 | 2.05 | 3.35 | 2.83 | 1.20 | 3.37 | 2.56 |
| 950836 | 34.79 | 13.60 | 2.31 | 1.27 | 6.82 | 0.77 | 1.62 | 2.56 |
| 461699 | 3.44 | 1.35 | 1.58 | 2.10 | 5.96 | 1.28 | 1.84 | 2.55 |
| 743851 | 8.22 | 3.22 | 3.05 | 2.50 | 2.28 | 3.39 | 1.54 | 2.55 |
| 731290 | 11.60 | 4.56 | 1.16 | 2.19 | 1.46 | 7.52 | 0.38 | 2.54 |
| 1475842 | 224.84 | 89.33 | 0.55 | 1.42 | 1.77 | 6.27 | 2.56 | 2.52 |
| 796513 | 17.83 | 7.09 | 3.08 | 2.43 | 2.08 | 3.84 | 1.14 | 2.51 |
| 82905 | 1.21 | 0.48 | 2.88 | 1.65 | 1.67 | 3.07 | 3.25 | 2.50 |
| 123755 | 5.06 | 2.03 | 3.88 | 1.17 | 1.06 | 1.14 | 5.22 | 2.49 |
| 884606 | 33.46 | 13.42 | 2.43 | 1.59 | 6.84 | 0.91 | 0.70 | 2.49 |
| 323611 | 1.25 | 0.50 | 3.60 | 1.64 | 1.66 | 2.19 | 3.33 | 2.48 |
| 1472150 | 35.19 | 14.23 | 3.57 | 1.68 | 1.35 | 4.43 | 1.33 | 2.47 |
| 950574 | 28.90 | 11.69 | 3.24 | 3.20 | 3.55 | 1.12 | 1.25 | 2.47 |
| 345056 | 0.87 | 0.35 | 1.32 | 3.06 | 3.37 | 2.76 | 1.82 | 2.47 |
| 487499 | 4.41 | 1.79 | 1.73 | 2.00 | 5.71 | 2.00 | 0.89 | 2.47 |
| 289071 | 37.48 | 15.32 | 1.87 | 2.50 | 5.68 | 1.17 | 1.01 | 2.45 |
| 1604342 | 1.97 | 0.81 | 1.10 | 1.51 | 0.94 | 6.33 | 2.34 | 2.45 |
| 896914 | 18.03 | 7.40 | 3.11 | 4.47 | 0.31 | 3.53 | 0.78 | 2.44 |
| 251330 | 9.21 | 3.78 | 1.73 | 1.78 | 2.05 | 3.30 | 3.33 | 2.44 |
| 470114 | 3.09 | 1.27 | 2.30 | 3.43 | 1.78 | 3.57 | 1.10 | 2.44 |
| 854678 | 6.21 | 2.55 | 1.30 | 7.14 | 1.43 | 0.90 | 1.41 | 2.44 |
| 245860 | 30.36 | 12.47 | 4.73 | 1.10 | 4.77 | 0.61 | 0.96 | 2.43 |
| 28436 | 0.89 | 0.37 | 1.13 | 1.64 | 1.72 | 5.29 | 2.37 | 2.43 |
| 344589 | 53.28 | 21.92 | 1.02 | 3.29 | 3.08 | 2.11 | 2.65 | 2.43 |
| 511952 | 225.66 | 92.92 | 0.39 | 2.42 | 0.61 | 7.65 | 1.07 | 2.43 |
| 898198 | 14.33 | 5.93 | 1.87 | 2.53 | 3.20 | 1.41 | 3.07 | 2.42 |
| 291706 | 2.41 | 1.00 | 1.70 | 1.35 | 1.24 | 1.16 | 6.59 | 2.41 |
| 258521 | 3.33 | 1.39 | 1.28 | 6.91 | 0.97 | 0.98 | 1.86 | 2.40 |
| 1412481 | 8.41 | 3.50 | 0.22 | 0.14 | 0.25 | 11.19 | 0.20 | 2.40 |
| 1475881 | 14.80 | 6.17 | 0.78 | 2.10 | 1.60 | 4.22 | 3.29 | 2.40 |
| 200599 | 13.42 | 5.61 | 3.26 | 1.35 | 4.93 | 0.84 | 1.59 | 2.39 |
| 252904 | 5.40 | 2.26 | 3.71 | 1.41 | 3.30 | 1.76 | 1.77 | 2.39 |
| 430614 | 2.38 | 1.00 | 2.34 | 3.41 | 1.17 | 4.02 | 0.99 | 2.39 |
| 381036 | 3.54 | 1.49 | 2.56 | 1.79 | 5.52 | 1.27 | 0.79 | 2.38 |
| 448117 | 3.91 | 1.64 | 3.86 | 1.93 | 3.01 | 1.46 | 1.64 | 2.38 |
| 250673 | 2.66 | 1.12 | 5.54 | 1.03 | 1.65 | 1.42 | 2.21 | 2.37 |
| 593929 | 17.74 | 7.51 | 3.61 | 1.43 | 5.14 | 0.93 | 0.70 | 2.36 |
| 841615 | 12.16 | 5.15 | 2.59 | 1.62 | 5.18 | 1.30 | 1.11 | 2.36 |
| 52338 | 2.91 | 1.24 | 3.59 | 1.69 | 4.60 | 0.65 | 1.22 | 2.35 |
| 137254 | 1.35 | 0.58 | 3.41 | 1.79 | 3.95 | 1.23 | 1.38 | 2.35 |
| 767994 | 4.29 | 1.83 | 2.20 | 1.22 | 7.00 | 0.55 | 0.73 | 2.34 |
| 197765 | 8.83 | 3.78 | 2.35 | 0.96 | 6.16 | 0.88 | 1.33 | 2.34 |
| 254310 | 10.26 | 4.39 | 2.47 | 1.99 | 5.33 | 1.07 | 0.80 | 2.33 |
| 855244 | 15.78 | 6.78 | 4.53 | 0.61 | 5.42 | 0.42 | 0.66 | 2.33 |
| 144926 | 9.20 | 3.96 | 3.10 | 1.65 | 2.25 | 3.17 | 1.43 | 2.32 |
| 565779 | 31.62 | 13.68 | 3.23 | 1.26 | 1.97 | 3.57 | 1.53 | 2.31 |
| 563451 | 4.17 | 1.82 | 3.24 | 2.08 | 3.69 | 1.02 | 1.43 | 2.29 |
| 462325 | 8.76 | 3.83 | 1.92 | 0.60 | 1.72 | 5.27 | 1.93 | 2.29 |
| 132911 | 52.56 | 22.96 | 3.65 | 1.43 | 4.04 | 0.88 | 1.45 | 2.29 |
| 827168 | 6.51 | 2.85 | 2.17 | 0.93 | 6.04 | 1.22 | 1.08 | 2.29 |
| 1070015 | 3.26 | 1.43 | 0.69 | 2.05 | 0.53 | 3.30 | 4.86 | 2.28 |
| 293243 | 13.17 | 5.84 | 3.23 | 1.48 | 4.46 | 0.69 | 1.43 | 2.26 |
| 743211 | 3.84 | 1.71 | 1.36 | 1.66 | 5.64 | 1.28 | 1.27 | 2.24 |
| 259462 | 3.69 | 1.65 | 0.66 | 0.89 | 0.75 | 8.24 | 0.65 | 2.24 |
| 291345 | 70.40 | 31.69 | 5.27 | 1.32 | 2.52 | 0.90 | 1.10 | 2.22 |
| 281191 | 7.36 | 3.32 | 3.42 | 1.94 | 3.36 | 0.84 | 1.52 | 2.22 |
| 362080 | 3.81 | 1.72 | 1.00 | 0.91 | 4.67 | 3.13 | 1.35 | 2.21 |
| 825058 | 3.42 | 1.55 | 2.14 | 0.93 | 6.34 | 1.08 | 0.54 | 2.21 |
| 290841 | 6.35 | 2.89 | 3.60 | 3.33 | 1.43 | 1.62 | 1.02 | 2.20 |
| 291057 | 1.34 | 0.61 | 1.18 | 1.53 | 3.04 | 4.12 | 1.04 | 2.18 |
| 855624 | 17.75 | 8.17 | 0.67 | 0.78 | 0.54 | 0.73 | 8.15 | 2.17 |
| 50344 | 0.85 | 0.39 | 0.80 | 0.85 | 0.73 | 7.58 | 0.85 | 2.16 |
| 1056172 | 10.33 | 4.78 | 2.40 | 1.52 | 0.48 | 5.66 | 0.74 | 2.16 |
| 1056203 | 1.92 | 0.89 | 1.05 | 0.95 | 1.43 | 6.28 | 1.07 | 2.15 |
| 786609 | 4.81 | 2.23 | 0.54 | 0.55 | 1.98 | 7.08 | 0.62 | 2.15 |
| 248261 | 1.51 | 0.70 | 3.67 | 1.05 | 0.77 | 0.89 | 4.36 | 2.15 |
| 429047 | 3.17 | 1.48 | 1.32 | 2.03 | 3.22 | 1.07 | 3.04 | 2.14 |
| 744933 | 2.15 | 1.01 | 0.80 | 0.91 | 0.71 | 7.21 | 1.06 | 2.14 |
| 461955 | 1.94 | 0.91 | 3.10 | 3.01 | 1.55 | 1.32 | 1.68 | 2.13 |
| 81484 | 10.87 | 5.15 | 1.73 | 3.05 | 2.09 | 0.66 | 3.02 | 2.11 |
| 897547 | 15.69 | 7.46 | 3.48 | 3.41 | 2.18 | 0.69 | 0.75 | 2.10 |
| 796366 | 34.94 | 17.03 | 3.14 | 1.59 | 3.66 | 0.93 | 0.94 | 2.05 |
| 141336 | 2.12 | 1.03 | 0.73 | 0.85 | 7.18 | 0.60 | 0.88 | 2.05 |
| 1291658 | 2.05 | 1.00 | 3.72 | 1.14 | 3.32 | 0.94 | 1.12 | 2.05 |
| 841302 | 7.41 | 3.64 | 1.25 | 1.26 | 5.92 | 0.84 | 0.91 | 2.04 |
| 627688 | 7.37 | 3.62 | 1.23 | 1.30 | 5.58 | 1.43 | 0.63 | 2.03 |
| 811097 | 2.59 | 1.28 | 5.58 | 1.38 | 0.73 | 1.83 | 0.60 | 2.02 |
| 361943 | 2.86 | 1.43 | 1.03 | 1.71 | 5.64 | 0.71 | 0.95 | 2.01 |
| 75059 | 21.82 | 10.94 | 3.07 | 1.03 | 1.07 | 3.28 | 1.53 | 2.00 |
| 282895 | 3.91 | 1.96 | 1.06 | 1.07 | 6.06 | 0.92 | 0.83 | 1.99 |
| 1492967 | 3.68 | 1.85 | 1.76 | 1.20 | 5.34 | 0.93 | 0.72 | 1.99 |
| 22600 | 0.36 | 0.18 | 1.38 | 5.49 | 0.87 | 1.20 | 1.00 | 1.99 |
| 119851 | 6.52 | 3.28 | 4.42 | 3.58 | 0.65 | 0.63 | 0.64 | 1.98 |
| 784285 | 3.99 | 2.02 | 3.44 | 1.06 | 3.08 | 1.24 | 1.07 | 1.98 |
| 140792 | 1.66 | 0.85 | 1.20 | 1.05 | 5.74 | 0.76 | 1.05 | 1.96 |
| 704076 | 5.70 | 2.95 | 1.28 | 1.04 | 3.20 | 3.25 | 0.88 | 1.93 |
| 142326 | 1.09 | 0.78 | 5.18 | 1.78 | 0.80 | 0.91 | 0.92 | 1.92 |
| 840944 | 79.86 | 42.51 | 3.82 | 0.54 | 4.16 | 0.61 | 0.27 | 1.88 |
| 377672 | 2.59 | 1.39 | 0.54 | 0.57 | 0.40 | 7.29 | 0.56 | 1.87 |
| 431597 | 5.45 | 2.95 | 1.55 | 0.82 | 5.32 | 0.67 | 0.90 | 1.85 |
| 32381 | 2.18 | 1.18 | 0.60 | 0.99 | 3.26 | 1.10 | 3.29 | 1.85 |
| 471641 | 4.79 | 2.74 | 0.55 | 3.60 | 0.72 | 3.36 | 0.52 | 1.75 |
| 753794 | 1.65 | 0.94 | 0.66 | 1.32 | 0.68 | 5.31 | 0.78 | 1.75 |
| 950464 | 9.75 | 5.67 | 1.05 | 0.71 | 5.37 | 0.77 | 0.68 | 1.72 |
| 289832 | 6.67 | 4.11 | 1.15 | 0.75 | 5.01 | 0.74 | 0.48 | 1.62 |
| 309864 | 2.49 | 1.62 | 5.57 | 0.48 | 0.62 | 0.55 | 0.47 | 1.54 |
| 1492202 | 2.60 | 1.98 | 0.42 | 0.35 | 0.43 | 0.34 | 5.02 | 1.31 |

TABLE 1-3

| | GenBank | Aver. of | Aver. of | Aver. | Tumor / | Tumor / | T/N | T/N | T/BPH | T/BPH |
|---|---|---|---|---|---|---|---|---|---|---|
| Clone | Acc No. | Tumors | Normals | of BPH | Normal | BPH | 5X, ≧1/5 | 3X, ≧2/5 | 5X, ≧1/5 | 3X, ≧2/5 |
| 788180 | AA453562 | 25.18 | 0.81 | 0.50 | 31.24 | 50.69 | + | + | + | + |
| 646037 | AA196979 | 24.42 | 1.02 | 0.75 | 23.91 | 32.70 | + | + | + | + |
| 1034473 | AA779728 | 33.61 | 1.51 | 1.35 | 22.27 | 24.97 | + | + | + | + |
| 281003 | N50880 | 128.77 | 6.30 | 4.90 | 20.43 | 26.26 | + | + | + | + |
| 308682 | W25202 | 8.99 | 0.87 | 0.27 | 10.37 | 32.91 | + | + | + | + |
| 281039 | N47717 | 14.05 | 0.94 | 0.90 | 14.99 | 15.57 | + | + | + | + |
| 284701 | N64840 | 33.05 | 7.05 | 1.52 | 4.68 | 21.68 | + | + | + | + |
| 823940 | AA490213 | 22.39 | 1.32 | 2.83 | 16.99 | 7.91 | + | + | + | + |
| 42415 | R60981 | 6.86 | 0.51 | 0.72 | 13.52 | 9.49 | + | + | + | + |
| 46166 | H09076 | 13.13 | 0.93 | 1.71 | 14.12 | 7.68 | + | + | + | + |
| 784168 | AA432103 | 53.04 | 7.37 | 3.83 | 7.20 | 13.84 | + | + | + | + |
| 773479 | AA427899 | 12.32 | 0.65 | 5.99 | 18.85 | 2.05 | + | + | − | − |
| 222157 | H85806 | 8.80 | 1.02 | 0.72 | 8.67 | 12.14 | + | + | + | + |
| 486035 | AA043109 | 24.75 | 3.47 | 1.86 | 7.13 | 13.32 | + | + | + | + |
| 450049 | AA703396 | 18.37 | 1.83 | 1.80 | 10.05 | 10.21 | + | + | + | + |
| 66336 | T66832 | 1.67 | 0.63 | 0.10 | 2.63 | 16.70 | − | + | + | + |
| 35058 | R45192 | 15.59 | 1.89 | 1.44 | 8.24 | 10.81 | + | + | + | + |
| 26736 | R39891 | 9.52 | 3.36 | 0.62 | 2.83 | 15.37 | + | + | + | + |
| 593185 | AA159714 | 28.06 | 5.61 | 2.29 | 5.00 | 12.23 | + | + | + | + |
| 32160 | R43432 | 26.80 | 3.59 | 2.77 | 7.47 | 9.69 | + | + | + | + |
| 814053 | AA465495 | 14.66 | 1.70 | 1.75 | 8.61 | 8.38 | + | + | + | + |
| 292515 | N68465 | 16.22 | 2.86 | 1.56 | 5.67 | 10.38 | + | + | + | + |
| 730739 | AA436097 | 36.49 | 7.10 | 3.45 | 5.14 | 10.57 | + | + | + | + |
| 282089 | N48259 | 11.18 | 1.51 | 1.41 | 7.41 | 7.92 | + | + | + | + |
| 283312 | N54763 | 63.94 | 6.63 | 12.14 | 9.65 | 5.27 | + | + | + | + |
| 291255 | N72215 | 15.20 | 1.35 | 4.65 | 11.29 | 3.27 | + | + | + | + |
| 1048810 | AA621342 | 20.15 | 3.04 | 2.59 | 6.63 | 7.78 | + | + | + | + |
| 197525 | H52119 | 9.52 | 1.85 | 1.06 | 5.16 | 9.01 | + | + | + | + |
| 67401 | T49326 | 1.47 | 0.20 | 0.23 | 7.28 | 6.27 | + | − | + | − |
| 490178 | AA121266 | 24.55 | 5.63 | 2.73 | 4.36 | 8.99 | + | − | + | + |
| 1556526 | AA935560 | 0.88 | 0.15 | 0.12 | 5.96 | 7.33 | + | − | + | − |
| 746152 | AA419486 | 9.35 | 2.23 | 1.07 | 4.18 | 8.70 | + | + | + | + |
| 1034738 | AA780190 | 22.40 | 3.90 | 3.28 | 5.74 | 6.82 | + | + | + | + |
| 259374 | N31952 | 10.51 | 2.33 | 1.31 | 4.50 | 8.04 | + | + | + | + |
| 757435 | AA437224 | 365.78 | 59.86 | 58.35 | 6.11 | 6.27 | + | + | + | + |
| 754280 | AA479284 | 23.83 | 3.20 | 4.92 | 7.46 | 4.85 | + | + | + | + |
| 877835 | AA625634 | 23.06 | 4.34 | 3.34 | 5.32 | 6.90 | + | + | + | + |
| 712950 | AA282272 | 11.53 | 3.21 | 1.34 | 3.59 | 8.58 | + | + | + | + |
| 50772 | H16803 | 18.10 | 6.74 | 1.91 | 2.68 | 9.47 | + | − | + | + |
| 1636908 | AI000271 | 6.58 | 4.38 | 0.63 | 1.50 | 10.44 | − | − | + | + |
| 432194 | AA679414 | 10.34 | 1.11 | 4.04 | 9.35 | 2.56 | + | + | − | + |
| 488964 | AA047260 | 7.77 | 2.13 | 0.96 | 3.64 | 8.08 | + | − | + | + |
| 1412481 | AA845156 | 8.41 | 3.50 | 0.90 | 2.40 | 9.31 | + | − | + | − |
| 283173 | N45236 | 17.85 | 3.97 | 2.47 | 4.49 | 7.21 | + | + | + | + |
| 811867 | AA454963 | 24.57 | 2.97 | 7.43 | 8.28 | 3.30 | + | + | − | + |
| 26519 | R20669 | 36.05 | 19.25 | 3.76 | 1.87 | 9.59 | − | − | + | + |
| 687297 | AA235224 | 12.18 | 3.12 | 1.63 | 3.90 | 7.48 | − | + | + | + |
| 666334 | AA232249 | 1.64 | 1.54 | 0.16 | 1.07 | 10.14 | − | − | + | + |
| 1468260 | AA884926 | 4.53 | 1.13 | 0.65 | 4.03 | 6.96 | + | + | + | + |
| 258300 | N30680 | 18.15 | 4.53 | 2.65 | 4.01 | 6.86 | + | + | + | + |
| 971279 | AA682905 | 54.34 | 6.63 | 22.20 | 8.20 | 2.45 | + | + | − | − |
| 1610448 | AA991856 | 135.55 | 57.06 | 16.44 | 2.38 | 8.25 | − | − | + | + |
| 461522 | AA705237 | 1.21 | 0.65 | 0.14 | 1.87 | 8.74 | − | − | + | + |
| 868282 | AA633957 | 8.99 | 2.01 | 1.47 | 4.48 | 6.12 | + | + | + | + |
| 280217 | N47941 | 20.52 | 5.86 | 2.89 | 3.50 | 7.09 | + | + | + | + |
| 240694 | H78135 | 4.53 | 0.90 | 0.83 | 5.01 | 5.47 | + | + | + | + |
| 321706 | W35398 | 7.80 | 1.86 | 1.25 | 4.20 | 6.23 | + | + | + | + |
| 770066 | AA430545 | 11.43 | 2.60 | 1.93 | 4.40 | 5.94 | + | + | + | + |
| 811603 | AI732784 | 19.62 | 2.74 | 6.22 | 7.16 | 3.15 | + | + | − | + |
| 51773 | H23211 | 11.48 | 2.40 | 2.09 | 4.77 | 5.50 | + | + | + | + |
| 66317 | T66815 | 5.50 | 1.76 | 0.78 | 3.13 | 7.06 | + | + | + | + |
| 35626 | R45292 | 9.42 | 1.09 | 6.51 | 8.67 | 1.45 | + | + | − | − |
| 450307 | AA682851 | 9.39 | 1.62 | 2.25 | 5.80 | 4.17 | + | + | + | + |
| 447715 | AA702781 | 14.83 | 7.21 | 1.88 | 2.06 | 7.90 | − | − | + | + |
| 731290 | AA416843 | 11.60 | 4.56 | 1.57 | 2.54 | 7.40 | + | − | + | + |
| 825857 | AA504783 | 4.27 | 2.51 | 0.52 | 1.70 | 8.20 | − | − | + | + |
| 565235 | AA136125 | 35.83 | 12.59 | 5.15 | 2.85 | 6.96 | + | + | + | + |
| 756600 | AA481464 | 47.06 | 7.13 | 14.79 | 6.60 | 3.18 | + | + | − | + |
| 293635 | N63807 | 26.47 | 3.44 | 12.94 | 7.69 | 2.05 | + | + | − | − |
| 757392 | AA426110 | 0.86 | 0.37 | 0.12 | 2.32 | 7.41 | − | − | + | + |
| 595695 | AA173189 | 3.58 | 2.33 | 0.44 | 1.54 | 8.16 | − | − | + | + |
| 151055 | H02230 | 27.32 | 7.51 | 4.62 | 3.64 | 5.92 | + | + | + | + |
| 297439 | W03687 | 48.60 | 11.33 | 9.26 | 4.29 | 5.25 | + | + | + | + |
| 838366 | AA457172 | 11.15 | 1.82 | 3.27 | 6.12 | 3.41 | + | + | − | + |
| 460279 | AA677629 | 0.87 | 0.46 | 0.11 | 1.87 | 7.58 | − | − | + | + |
| 595297 | AA173598 | 3.07 | 2.45 | 0.38 | 1.25 | 8.17 | − | − | + | + |
| 1631713 | AI025015 | 5.68 | 0.93 | 1.77 | 6.13 | 3.21 | + | + | − | + |
| 564801 | AA129551 | 12.35 | 3.96 | 1.99 | 3.12 | 6.21 | + | − | + | + |
| 293651 | N69653 | 13.35 | 1.84 | 6.56 | 7.26 | 2.04 | + | + | − | − |
| 745347 | AA625666 | 2.15 | 0.29 | 1.14 | 7.39 | 1.88 | + | + | − | − |
| 772304 | AA404486 | 113.14 | 18.10 | 38.24 | 6.25 | 2.96 | + | + | − | + |
| 262053 | N25030 | 10.75 | 5.15 | 1.52 | 2.09 | 7.08 | − | − | + | + |
| 81331 | T60075 | 2.84 | 0.78 | 0.51 | 3.63 | 5.53 | + | − | + | + |
| 840894 | AA482340 | 34.81 | 11.87 | 5.66 | 2.93 | 6.15 | − | + | + | + |
| 839081 | AA488732 | 16.60 | 5.37 | 2.78 | 3.09 | 5.98 | + | + | + | + |
| 417908 | W90519 | 12.10 | 2.57 | 2.82 | 4.71 | 4.29 | + | + | + | + |
| 796366 | AA459761 | 34.94 | 17.03 | 5.05 | 2.05 | 6.92 | − | + | + | + |
| 359661 | AA011095 | 26.24 | 8.79 | 4.39 | 2.99 | 5.97 | − | + | + | + |
| 50188 | H17943 | 10.75 | 2.29 | 2.53 | 4.69 | 4.25 | + | + | − | + |
| 84955 | T74846 | 5.26 | 1.01 | 1.43 | 5.21 | 3.69 | + | + | + | + |
| 159608 | H16152 | 14.43 | 4.14 | 2.69 | 3.49 | 5.36 | + | + | + | + |
| 1390584 | AA843592 | 6.54 | 1.91 | 1.21 | 3.42 | 5.41 | + | + | + | + |
| 825736 | AA504842 | 0.92 | 0.60 | 0.13 | 1.54 | 7.26 | − | − | + | + |
| 22161 | T66154 | 20.24 | 13.40 | 2.80 | 1.51 | 7.24 | − | − | + | + |
| 787938 | AA452278 | 5.09 | 1.27 | 1.08 | 4.01 | 4.71 | + | + | + | + |
| 809603 | AA442991 | 49.03 | 6.94 | 29.52 | 7.06 | 1.66 | + | + | − | − |
| 826109 | AA521327 | 0.75 | 0.54 | 0.10 | 1.38 | 7.27 | − | − | + | + |
| 884606 | AA630006 | 33.46 | 13.42 | 5.43 | 2.49 | 6.16 | + | − | + | + |
| 825822 | AA491370 | 14.07 | 9.48 | 1.97 | 1.48 | 7.16 | − | − | + | + |
| 531028 | AA070495 | 21.14 | 3.89 | 6.62 | 5.44 | 3.20 | + | + | − | + |
| 814017 | AA455668 | 86.17 | 23.42 | 17.57 | 3.68 | 4.90 | + | + | + | + |
| 814062 | AA465338 | 0.81 | 0.47 | 0.12 | 1.72 | 6.83 | − | − | + | + |
| 826103 | AA521416 | 1.31 | 0.82 | 0.19 | 1.61 | 6.93 | − | − | + | + |
| 461670 | AA779919 | 8.90 | 1.72 | 2.71 | 5.19 | 3.28 | + | + | − | + |
| 415700 | W85697 | 3.36 | 0.61 | 1.15 | 5.53 | 2.93 | + | + | − | + |
| 815163 | AA481144 | 1.20 | 0.57 | 0.19 | 2.09 | 6.36 | − | − | + | + |
| 810558 | AA464568 | 14.49 | 3.00 | 4.01 | 4.83 | 3.61 | + | + | + | + |
| 810220 | AA464689 | 2.17 | 0.39 | 0.77 | 5.60 | 2.82 | + | + | − | + |
| 359781 | AA010932 | 7.82 | 2.50 | 1.49 | 3.13 | 5.26 | + | + | + | + |
| 795265 | AA451807 | 4.58 | 1.26 | 0.97 | 3.63 | 4.74 | + | − | + | + |
| 840306 | AA485333 | 2.49 | 1.26 | 0.39 | 1.97 | 6.38 | − | − | + | + |
| 212165 | H68845 | 41.96 | 8.42 | 12.58 | 4.99 | 3.34 | + | + | + | + |
| 138444 | R68272 | 4.04 | 1.00 | 0.95 | 4.06 | 4.24 | + | + | + | + |
| 824723 | AA488986 | 14.11 | 4.37 | 2.84 | 3.23 | 4.96 | + | + | + | + |
| 186918 | H43317 | 3.04 | 1.10 | 0.56 | 2.76 | 5.38 | + | − | + | + |
| 191904 | H38880 | 7.67 | 1.44 | 2.75 | 5.32 | 2.79 | + | + | − | + |
| 1630990 | AI018613 | 20.64 | 7.84 | 3.79 | 2.63 | 5.44 | − | + | + | + |
| 950688 | AA608567 | 52.65 | 11.99 | 14.32 | 4.39 | 3.68 | + | + | + | + |
| 771317 | AA476234 | 9.44 | 1.78 | 3.46 | 5.31 | 2.73 | + | + | − | − |
| 826072 | AA521394 | 1.20 | 0.83 | 0.18 | 1.44 | 6.59 | − | − | + | + |
| 30428 | R42061 | 4.29 | 0.99 | 1.16 | 4.35 | 3.68 | + | + | + | + |
| 1056172 | AA620995 | 10.33 | 4.78 | 1.77 | 2.16 | 5.84 | + | − | + | + |
| 251806 | H96647 | 2.94 | 1.83 | 0.46 | 1.60 | 6.39 | − | − | + | + |
| 305538 | N89861 | 11.44 | 3.31 | 2.54 | 3.46 | 4.50 | − | + | + | + |
| 52933 | H29315 | 3.59 | 1.99 | 0.58 | 1.80 | 6.14 | − | − | + | + |
| 587415 | AI733857 | 12.98 | 3.44 | 3.12 | 3.77 | 4.16 | + | + | + | + |
| 824510 | AA490522 | 1.38 | 1.09 | 0.21 | 1.27 | 6.65 | − | − | + | + |
| 416436 | W86823 | 11.08 | 2.37 | 3.42 | 4.67 | 3.24 | + | + | + | + |
| 321470 | W44889 | 7.74 | 1.68 | 2.36 | 4.61 | 3.29 | + | + | − | + |
| 842925 | AA486444 | 4.61 | 1.21 | 1.13 | 3.79 | 4.07 | − | + | − | + |
| 859228 | AA666366 | 85.06 | 37.74 | 15.20 | 2.25 | 5.60 | − | − | + | + |
| 845363 | AA644092 | 29.59 | 7.07 | 8.08 | 4.18 | 3.66 | + | + | + | + |
| 362755 | AA018675 | 16.82 | 3.11 | 6.91 | 5.41 | 2.43 | + | + | − | − |
| 253132 | H89036 | 1.40 | 1.21 | 0.21 | 1.16 | 6.66 | − | − | + | + |
| 233419 | H77706 | 5.55 | 1.14 | 1.90 | 4.87 | 2.93 | + | + | − | + |
| 1592006 | AA953229 | 5.30 | 1.51 | 1.24 | 3.52 | 4.28 | − | + | − | + |
| 950778 | AA608636 | 3.00 | 1.87 | 0.48 | 1.60 | 6.18 | − | − | + | + |
| 951313 | AA620591 | 6.84 | 6.68 | 1.01 | 1.03 | 6.75 | − | − | + | + |
| 358850 | W94647 | 21.29 | 5.98 | 5.06 | 3.56 | 4.21 | + | + | + | + |
| 771048 | AA427395 | 10.54 | 2.90 | 2.58 | 3.63 | 4.08 | − | + | + | + |
| 840944 | AA486533 | 79.86 | 42.51 | 13.69 | 1.88 | 5.83 | − | + | + | + |
| 268211 | N36327 | 3.43 | 0.70 | 1.23 | 4.93 | 2.78 | + | + | + | − |
| 840606 | AA487914 | 15.85 | 7.18 | 2.88 | 2.21 | 5.50 | − | − | + | + |
| 796161 | AA461092 | 30.31 | 5.27 | 15.52 | 5.75 | 1.95 | + | + | − | − |
| 266106 | N21624 | 20.68 | 4.53 | 6.63 | 4.57 | 3.12 | + | + | + | + |
| 811582 | AA458516 | 10.13 | 5.34 | 1.75 | 1.90 | 5.78 | − | − | + | + |
| 741067 | AA402352 | 64.30 | 16.50 | 17.00 | 3.90 | 3.78 | + | + | + | + |
| 214006 | H70775 | 4.67 | 1.55 | 1.00 | 3.02 | 4.66 | + | + | + | + |
| 449112 | AA777488 | 42.54 | 7.72 | 19.71 | 5.51 | 2.16 | + | + | − | − |
| 150262 | H01129 | 13.66 | 3.31 | 3.87 | 4.12 | 3.53 | + | + | − | + |
| 289891 | N77134 | 7.82 | 1.84 | 2.30 | 4.25 | 3.40 | + | + | + | + |
| 488431 | AA047441 | 10.48 | 3.68 | 2.19 | 2.85 | 4.78 | − | + | + | + |
| 788655 | AA449837 | 7.66 | 2.78 | 1.58 | 2.76 | 4.86 | − | + | + | + |
| 208082 | H62563 | 7.90 | 1.98 | 2.19 | 3.99 | 3.60 | + | + | + | + |
| 745503 | AA625995 | 42.62 | 11.14 | 11.32 | 3.83 | 3.77 | + | + | + | + |
| 509731 | AA045699 | 39.41 | 23.94 | 6.63 | 1.65 | 5.95 | − | − | + | + |
| 565863 | AA136386 | 3.93 | 2.97 | 0.63 | 1.32 | 6.27 | − | − | + | + |
| 509887 | AA054701 | 39.47 | 7.39 | 17.54 | 5.34 | 2.25 | + | + | − | − |
| 796646 | AA461467 | 6.21 | 4.35 | 1.01 | 1.43 | 6.16 | − | − | + | + |
| 814235 | AA465598 | 12.37 | 6.41 | 2.19 | 1.93 | 5.65 | − | − | + | + |
| 53022 | R15880 | 1.34 | 0.70 | 0.24 | 1.90 | 5.63 | − | − | + | + |
| 377246 | AA055306 | 12.60 | 3.02 | 3.78 | 4.17 | 3.34 | + | + | + | − |
| 814662 | AA481045 | 1.12 | 0.67 | 0.19 | 1.67 | 5.84 | − | − | + | + |
| 45327 | H08548 | 8.18 | 2.30 | 2.08 | 3.56 | 3.94 | − | + | + | + |
| 51700 | H22853 | 3.66 | 1.21 | 0.82 | 3.02 | 4.47 | − | + | + | + |
| 80050 | T63893 | 8.15 | 3.84 | 1.52 | 2.12 | 5.35 | − | − | + | + |
| 594994 | AA172372 | 14.61 | 10.98 | 2.38 | 1.33 | 6.14 | − | − | + | + |
| 795907 | AA460346 | 5.39 | 1.25 | 1.71 | 4.31 | 3.15 | + | + | + | + |
| 824126 | AA490611 | 0.59 | 0.37 | 0.10 | 1.58 | 5.87 | − | − | + | + |
| 511952 | AA100674 | 225.66 | 92.92 | 45.17 | 2.43 | 5.00 | + | − | + | + |
| 823615 | AA496957 | 9.40 | 3.54 | 1.97 | 2.65 | 4.76 | + | + | + | + |
| 784130 | AA432075 | 17.23 | 6.93 | 3.50 | 2.49 | 4.93 | − | − | + | + |
| 1535596 | AA918370 | 401.67 | 285.67 | 66.91 | 1.41 | 6.00 | − | − | + | + |
| 1605407 | AA988746 | 9.00 | 4.61 | 1.65 | 1.95 | 5.45 | − | − | + | + |
| 1606837 | AA996028 | 23.76 | 9.09 | 4.98 | 2.61 | 4.77 | − | + | + | + |
| 768395 | AA495819 | 14.29 | 3.54 | 4.28 | 4.03 | 3.34 | + | + | − | + |
| 796239 | AA460675 | 26.07 | 23.22 | 4.18 | 1.12 | 6.24 | − | − | + | + |
| 49302 | H15675 | 4.88 | 0.86 | 2.90 | 5.68 | 1.68 | + | + | − | − |
| 754358 | AA436142 | 12.77 | 7.46 | 2.26 | 1.71 | 5.64 | − | − | + | + |
| 207029 | R98842 | 81.88 | 14.79 | 45.41 | 5.54 | 1.80 | + | + | − | − |
| 838285 | AA458747 | 1.34 | 1.36 | 0.21 | 0.99 | 6.35 | − | − | + | + |
| 1636108 | AI015679 | 3.58 | 1.45 | 0.74 | 2.47 | 4.85 | − | − | + | + |
| 796754 | AA460719 | 0.66 | 0.83 | 0.10 | 0.80 | 6.50 | − | − | + | + |
| 238821 | H65029 | 6.54 | 2.22 | 1.50 | 2.94 | 4.35 | + | + | + | + |
| 129024 | R10378 | 4.30 | 1.04 | 1.36 | 4.13 | 3.16 | + | + | + | + |
| 771053 | AA430718 | 16.14 | 6.63 | 3.34 | 2.43 | 4.83 | − | − | + | + |
| 289071 | N63609 | 37.48 | 15.32 | 7.78 | 2.45 | 4.82 | + | − | + | + |
| 1637282 | AI005515 | 4.01 | 2.58 | 0.70 | 1.55 | 5.71 | − | − | + | + |
| 263697 | H99672 | 4.23 | 0.93 | 1.58 | 4.57 | 2.68 | + | + | − | + |
| 971367 | AA683050 | 35.22 | 12.06 | 8.15 | 2.92 | 4.32 | + | + | + | + |
| 32381 | R43452 | 2.18 | 1.18 | 0.41 | 1.85 | 5.38 | − | + | + | + |
| 586780 | AA130677 | 1.92 | 1.75 | 0.31 | 1.10 | 6.12 | − | − | + | + |
| 815036 | AA465148 | 1.62 | 0.58 | 0.37 | 2.81 | 4.40 | − | + | + | + |
| 383185 | AA074258 | 12.04 | 3.46 | 3.22 | 3.48 | 3.73 | + | + | + | + |
| 416374 | W86202 | 4.49 | 2.66 | 0.81 | 1.69 | 5.52 | − | − | + | + |
| 781366 | AA448402 | 12.06 | 3.84 | 2.97 | 3.14 | 4.05 | + | + | + | + |
| 213682 | H71713 | 13.07 | 4.10 | 3.27 | 3.19 | 3.99 | + | + | + | + |
| 1467207 | AA884713 | 3.68 | 0.99 | 1.07 | 3.72 | 3.44 | − | + | − | + |
| 375682 | AA032221 | 31.04 | 12.66 | 6.60 | 2.45 | 4.70 | − | − | + | + |
| 510576 | AA055880 | 132.22 | 56.58 | 27.52 | 2.34 | 4.81 | − | − | + | + |
| 242952 | H95712 | 16.94 | 4.39 | 5.16 | 3.86 | 3.28 | − | + | − | + |
| 878127 | AA775422 | 3.53 | 0.87 | 1.16 | 4.07 | 3.05 | + | + | − | + |
| 853968 | AI791173 | 12.10 | 2.86 | 4.21 | 4.23 | 2.87 | + | + | + | + |
| 287728 | N75893 | 2.76 | 0.70 | 0.89 | 3.97 | 3.10 | + | + | + | + |
| 842927 | AA486445 | 4.24 | 3.59 | 0.72 | 1.18 | 5.88 | − | − | + | + |
| 897042 | AA676765 | 4.95 | 1.36 | 1.45 | 3.63 | 3.42 | + | + | + | + |
| 878281 | AA670286 | 6.80 | 3.30 | 1.37 | 2.06 | 4.97 | − | − | + | + |
| 359701 | AA011176 | 5.21 | 2.32 | 1.09 | 2.25 | 4.78 | − | − | + | + |
| 272750 | N36233 | 20.57 | 6.04 | 5.70 | 3.40 | 3.61 | + | + | + | + |
| 823819 | AA490390 | 11.96 | 3.51 | 3.34 | 3.41 | 3.58 | − | + | − | + |
| 897807 | AA598531 | 5.08 | 2.77 | 0.98 | 1.83 | 5.16 | − | − | + | + |
| 855749 | AA663983 | 50.50 | 12.03 | 18.24 | 4.20 | 2.77 | + | + | − | − |
| 264895 | N21170 | 7.86 | 1.63 | 3.70 | 4.82 | 2.12 | + | + | − | − |
| 825207 | AA504120 | 11.96 | 2.92 | 4.19 | 4.09 | 2.86 | + | + | − | − |
| 461476 | AA705041 | 0.78 | 0.61 | 0.14 | 1.28 | 5.64 | − | − | + | + |
| 323474 | W45623 | 1.25 | 0.32 | 0.41 | 3.87 | 3.06 | + | + | + | + |
| 744846 | AA625675 | 2.69 | 0.70 | 0.87 | 3.83 | 3.10 | + | + | − | + |
| 741474 | AA401111 | 4.93 | 1.40 | 1.45 | 3.51 | 3.40 | + | + | + | + |
| 506564 | AA709044 | 12.93 | 4.03 | 3.49 | 3.21 | 3.70 | − | + | + | + |
| 449275 | AA777705 | 3.81 | 0.83 | 1.64 | 4.58 | 2.32 | + | + | + | − |
| 878544 | AA775863 | 19.20 | 6.78 | 4.73 | 2.83 | 4.06 | + | + | + | + |
| 755239 | AA422139 | 17.93 | 9.29 | 3.62 | 1.93 | 4.95 | − | − | + | + |
| 259973 | N47829 | 1.66 | 1.95 | 0.28 | 0.85 | 5.99 | − | − | + | + |
| 290370 | N62301 | 0.62 | 0.48 | 0.11 | 1.30 | 5.53 | − | − | + | + |
| 773287 | AA425534 | 15.94 | 8.17 | 3.27 | 1.95 | 4.87 | − | − | + | + |
| 753252 | AA406505 | 7.78 | 2.14 | 2.45 | 3.63 | 3.18 | − | + | − | + |
| 290841 | N99711 | 6.35 | 2.89 | 1.38 | 2.20 | 4.61 | − | + | + | + |
| 50606 | H17763 | 14.94 | 6.00 | 3.47 | 2.49 | 4.31 | − | − | + | + |
| 1472689 | AA873159 | 5.15 | 1.21 | 2.03 | 4.26 | 2.53 | + | + | + | − |
| 252314 | H87175 | 0.69 | 0.62 | 0.12 | 1.11 | 5.66 | − | − | + | + |
| 813275 | AA455940 | 1.75 | 0.86 | 0.37 | 2.04 | 4.73 | − | − | + | + |
| 951125 | AA620556 | 18.86 | 7.00 | 4.63 | 2.69 | 4.07 | + | − | + | + |
| 454317 | AA677165 | 20.56 | 33.95 | 3.34 | 0.61 | 6.16 | − | − | + | + |
| 433253 | AA699427 | 7.29 | 2.43 | 1.95 | 3.00 | 3.74 | + | + | + | + |
| 1585549 | AA976714 | 6.44 | 3.55 | 1.31 | 1.81 | 4.92 | − | − | + | + |
| 491764 | AA150502 | 0.81 | 0.59 | 0.15 | 1.36 | 5.37 | − | − | + | + |
| 587595 | AA133044 | 559.22 | 179.15 | 155.61 | 3.12 | 3.59 | + | + | + | + |
| 245547 | N55167 | 5.86 | 1.63 | 1.88 | 3.59 | 3.12 | + | + | + | + |
| 200599 | H48472 | 13.42 | 5.61 | 3.11 | 2.39 | 4.32 | − | + | + | + |
| 139771 | R62352 | 7.72 | 6.17 | 1.42 | 1.25 | 5.44 | − | − | + | + |
| 462097 | AA705376 | 8.32 | 6.33 | 1.55 | 1.31 | 5.38 | − | − | + | + |
| 810571 | AA464580 | 4.37 | 1.58 | 1.11 | 2.76 | 3.92 | − | + | + | + |
| 279482 | N48804 | 8.66 | 3.25 | 2.16 | 2.67 | 4.00 | + | − | + | + |
| 127396 | R08658 | 14.07 | 5.12 | 3.61 | 2.75 | 3.90 | − | + | + | + |
| 824122 | AA490609 | 4.64 | 2.75 | 0.94 | 1.69 | 4.95 | − | − | + | + |
| 1031583 | AA609323 | 38.01 | 9.06 | 15.59 | 4.20 | 2.44 | + | + | + | − |
| 700900 | AA287375 | 9.94 | 3.00 | 3.00 | 3.31 | 3.31 | − | + | − | + |
| 250869 | N23605 | 123.92 | 32.54 | 44.10 | 3.81 | 2.81 | + | − | + | − |
| 1631747 | AI025120 | 5.09 | 1.33 | 1.84 | 3.84 | 2.77 | + | + | − | + |
| 896914 | AA779391 | 18.03 | 7.40 | 4.35 | 2.44 | 4.14 | − | + | + | + |
| 743452 | AA609368 | 7.90 | 4.94 | 1.59 | 1.60 | 4.98 | − | − | + | + |
| 81578 | T65844 | 12.77 | 4.82 | 3.25 | 2.65 | 3.93 | − | + | + | + |
| 837904 | AA434360 | 103.13 | 30.63 | 32.18 | 3.37 | 3.20 | + | + | + | + |
| 1030855 | AA621761 | 14.86 | 3.64 | 5.98 | 4.08 | 2.48 | + | + | − | − |
| 814306 | AA459100 | 18.28 | 10.68 | 3.77 | 1.71 | 4.85 | − | − | + | + |
| 77915 | T61271 | 14.99 | 17.48 | 2.63 | 0.86 | 5.70 | − | − | + | + |
| 260116 | N32044 | 0.88 | 0.86 | 0.16 | 1.03 | 5.52 | − | − | + | + |
| 811953 | AA458551 | 17.84 | 5.98 | 5.02 | 2.98 | 3.55 | − | + | − | + |
| 210405 | H65395 | 34.02 | 7.84 | 15.49 | 4.34 | 2.20 | + | + | − | − |
| 124824 | R01139 | 30.85 | 11.28 | 8.13 | 2.73 | 3.79 | + | − | + | + |
| 593929 | AA169379 | 17.74 | 7.51 | 4.26 | 2.36 | 4.17 | + | + | + | + |
| 703943 | AA278473 | 4.77 | 2.81 | 0.99 | 1.70 | 4.82 | − | − | + | + |
| 487733 | AA058818 | 15.01 | 6.81 | 3.48 | 2.20 | 4.32 | − | − | + | + |
| 191603 | H38210 | 22.14 | 4.64 | 12.72 | 4.77 | 1.74 | + | + | − | − |
| 360885 | AA012866 | 40.29 | 13.93 | 11.17 | 2.89 | 3.61 | − | + | + | + |
| 824267 | AA491265 | 0.74 | 0.41 | 0.16 | 1.81 | 4.68 | − | − | + | + |
| 624271 | AA181179 | 5.74 | 2.03 | 1.58 | 2.82 | 3.64 | + | + | + | + |
| 1500000 | AA885642 | 6.11 | 3.34 | 1.32 | 1.83 | 4.63 | − | − | + | + |
| 813973 | AA455689 | 3.24 | 0.88 | 1.18 | 3.70 | 2.75 | − | + | − | − |
| 825297 | AA504536 | 0.96 | 0.65 | 0.19 | 1.48 | 4.97 | − | − | + | + |
| 592403 | AA159497 | 0.46 | 0.16 | 0.13 | 2.95 | 3.50 | + | − | + | + |
| 838807 | AA457681 | 1.90 | 2.58 | 0.33 | 0.74 | 5.71 | − | − | + | + |
| 845352 | AA644080 | 13.36 | 5.39 | 3.37 | 2.48 | 3.97 | − | − | + | + |
| 814798 | AA465614 | 32.86 | 15.63 | 7.58 | 2.10 | 4.34 | − | − | + | + |
| 1607765 | AA989473 | 3.96 | 1.73 | 0.96 | 2.29 | 4.14 | − | − | + | + |
| 1035765 | AA629117 | 6.21 | 2.41 | 1.61 | 2.57 | 3.86 | + | + | + | + |
| 824739 | AA488784 | 2.63 | 1.02 | 0.68 | 2.57 | 3.86 | − | − | + | + |
| 290642 | N99659 | 7.76 | 2.56 | 2.28 | 3.02 | 3.40 | − | + | − | + |
| 121489 | T97303 | 34.70 | 10.55 | 11.09 | 3.29 | 3.13 | − | + | − | + |
| 823691 | AA489647 | 7.42 | 4.65 | 1.54 | 1.59 | 4.82 | − | − | + | + |
| 86160 | T12336 | 2.10 | 0.74 | 0.59 | 2.82 | 3.58 | − | + | − | + |
| 1456160 | AA862465 | 41.61 | 40.32 | 7.76 | 1.03 | 5.36 | − | − | + | + |
| 897767 | AA598470 | 2.07 | 0.49 | 0.98 | 4.25 | 2.12 | + | + | − | − |
| 877641 | AA488238 | 67.93 | 15.10 | 36.21 | 4.50 | 1.88 | + | + | − | − |
| 504544 | AA149096 | 12.31 | 4.73 | 3.28 | 2.60 | 3.76 | + | − | + | + |
| 1466631 | AA883679 | 1.21 | 0.71 | 0.26 | 1.71 | 4.63 | − | − | + | + |
| 898109 | AA598483 | 6.77 | 3.15 | 1.62 | 2.15 | 4.19 | − | − | + | + |
| 280763 | N50563 | 0.53 | 0.50 | 0.10 | 1.05 | 5.28 | − | − | + | + |
| 1492238 | AA875936 | 13.96 | 4.83 | 4.08 | 2.89 | 3.43 | − | + | − | + |
| 194364 | H50770 | 5.44 | 1.19 | 3.10 | 4.56 | 1.76 | + | + | − | − |
| 462468 | AA699895 | 3.24 | 0.96 | 1.10 | 3.36 | 2.96 | − | + | − | + |
| 768643 | AA430351 | 41.57 | 23.92 | 9.10 | 1.74 | 4.57 | − | − | + | + |
| 29063 | R40970 | 3.34 | 1.66 | 0.78 | 2.01 | 4.29 | − | − | + | + |
| 610124 | AA171459 | 1.70 | 1.73 | 0.32 | 0.98 | 5.32 | − | − | + | + |
| 26617 | R39862 | 4.62 | 2.68 | 1.01 | 1.73 | 4.56 | − | − | + | + |
| 487499 | AA045115 | 4.41 | 1.79 | 1.16 | 2.47 | 3.82 | + | − | + | + |
| 1631849 | AI004315 | 25.01 | 12.24 | 5.91 | 2.04 | 4.24 | − | − | + | + |
| 204111 | H55907 | 3.38 | 0.87 | 1.40 | 3.86 | 2.42 | + | + | − | − |
| 781454 | AA432312 | 9.75 | 3.24 | 2.99 | 3.01 | 3.26 | + | − | + | − |
| 491184 | AA137144 | 6.35 | 1.83 | 2.27 | 3.47 | 2.80 | + | + | + | + |
| 840788 | AA486145 | 23.03 | 5.46 | 11.26 | 4.22 | 2.05 | + | + | − | − |
| 547058 | AA082943 | 9.57 | 4.00 | 2.48 | 2.39 | 3.86 | − | − | − | + |
| 784253 | AA446906 | 11.76 | 3.47 | 4.12 | 3.39 | 2.85 | − | + | − | + |
| 196189 | R91950 | 11.19 | 4.82 | 2.86 | 2.32 | 3.91 | − | − | − | + |
| 47542 | H16454 | 8.07 | 1.88 | 4.18 | 4.30 | 1.93 | + | + | − | − |
| 47384 | H10778 | 4.22 | 1.17 | 1.61 | 3.61 | 2.62 | − | + | − | − |
| 824237 | AA491249 | 74.89 | 31.45 | 19.54 | 2.38 | 3.83 | − | − | + | + |
| 194723 | R89849 | 5.22 | 3.86 | 1.07 | 1.35 | 4.86 | − | − | + | + |
| 150314 | H01205 | 5.29 | 2.31 | 1.35 | 2.29 | 3.92 | − | − | + | + |
| 842784 | AA486200 | 326.54 | 118.59 | 94.88 | 2.75 | 3.44 | + | + | + | + |
| 48286 | H12190 | 2.34 | 0.61 | 0.99 | 3.83 | 2.36 | − | + | − | − |
| 454771 | AA677287 | 2.69 | 0.70 | 1.13 | 3.82 | 2.38 | + | + | − | − |
| 24208 | R38412 | 7.49 | 3.51 | 1.85 | 2.13 | 4.05 | − | − | + | + |
| 273546 | N33274 | 9.10 | 3.09 | 2.81 | 2.95 | 3.23 | − | + | − | + |
| 267419 | N24973 | 0.97 | 0.96 | 0.19 | 1.01 | 5.16 | − | − | + | + |
| 731410 | AA412250 | 0.53 | 0.58 | 0.10 | 0.92 | 5.24 | − | − | + | + |
| 1589973 | AA977296 | 5.17 | 1.57 | 1.81 | 3.30 | 2.85 | + | + | − | + |
| 825668 | AA505063 | 2.26 | 0.96 | 0.60 | 2.36 | 3.77 | − | − | − | + |
| 262864 | N24437 | 36.97 | 16.43 | 9.52 | 2.25 | 3.88 | − | − | + | + |
| 1055201 | AA626146 | 30.25 | 19.90 | 6.55 | 1.52 | 4.62 | − | − | + | + |
| 869450 | AA680244 | 51.58 | 35.87 | 10.98 | 1.44 | 4.70 | − | − | + | + |
| 37980 | R61372 | 0.84 | 0.29 | 0.27 | 2.96 | 3.18 | + | − | + | − |
| 124252 | R02439 | 6.06 | 4.74 | 1.25 | 1.28 | 4.85 | − | − | + | + |
| 238661 | H81543 | 2.74 | 2.18 | 0.56 | 1.26 | 4.86 | − | − | + | + |
| 593658 | AA160080 | 1.08 | 1.02 | 0.21 | 1.07 | 5.04 | − | − | + | + |
| 785616 | AA449004 | 11.47 | 3.12 | 4.70 | 3.67 | 2.44 | − | + | − | − |
| 84022 | T70901 | 6.97 | 2.71 | 1.97 | 2.57 | 3.54 | − | − | + | + |
| 511632 | AA126951 | 7.85 | 2.71 | 2.46 | 2.89 | 3.20 | − | + | − | + |
| 34134 | R44734 | 2.73 | 1.33 | 0.68 | 2.06 | 4.03 | − | − | + | + |
| 39885 | R52543 | 0.94 | 0.30 | 0.31 | 3.11 | 2.98 | − | + | − | + |
| 840590 | AA488014 | 21.70 | 7.54 | 6.78 | 2.88 | 3.20 | + | − | + | − |
| 49710 | H28973 | 3.21 | 1.47 | 0.82 | 2.18 | 3.89 | − | − | − | + |
| 42803 | R60014 | 2.96 | 0.81 | 1.23 | 3.66 | 2.41 | + | + | + | − |
| 134969 | R31789 | 6.81 | 4.59 | 1.48 | 1.48 | 4.58 | − | − | + | + |
| 75415 | T57609 | 51.71 | 24.46 | 13.09 | 2.11 | 3.95 | − | − | − | + |
| 843094 | AA488626 | 49.18 | 13.00 | 21.59 | 3.78 | 2.28 | − | + | − | − |
| 137189 | R36409 | 5.26 | 1.96 | 1.56 | 2.68 | 3.37 | − | + | − | + |
| 45578 | H08016 | 5.05 | 1.63 | 1.72 | 3.10 | 2.94 | − | + | − | + |
| 430465 | AA680186 | 5.55 | 1.53 | 2.30 | 3.62 | 2.41 | + | − | + | − |
| 67654 | T49539 | 15.79 | 6.24 | 4.50 | 2.53 | 3.51 | − | − | + | + |
| 550141 | AA102591 | 78.20 | 22.40 | 30.93 | 3.49 | 2.53 | + | + | − | − |
| 796351 | AA459765 | 2.34 | 1.80 | 0.50 | 1.30 | 4.72 | − | − | + | + |
| 1637296 | AI005519 | 25.52 | 13.27 | 6.23 | 1.92 | 4.09 | − | − | + | + |
| 549101 | AA083485 | 12.81 | 2.43 | 17.02 | 5.26 | 0.75 | + | + | − | − |
| 824376 | AA489696 | 0.49 | 0.34 | 0.11 | 1.42 | 4.58 | − | − | + | + |
| 773204 | AA425692 | 21.34 | 15.28 | 4.63 | 1.40 | 4.61 | − | − | + | + |
| 453599 | AA679569 | 4.23 | 2.23 | 1.03 | 1.89 | 4.10 | − | − | + | + |
| 815230 | AA481256 | 9.59 | 4.44 | 2.50 | 2.16 | 3.84 | − | − | + | + |
| 196650 | R93237 | 10.93 | 6.59 | 2.52 | 1.66 | 4.33 | − | − | + | + |
| 725489 | AA398521 | 26.71 | 9.34 | 8.54 | 2.86 | 3.13 | − | + | − | + |
| 1475987 | AA872436 | 2.96 | 0.83 | 1.23 | 3.58 | 2.40 | + | + | − | − |
| 811162 | AA485748 | 6.36 | 4.30 | 1.41 | 1.48 | 4.50 | − | − | + | + |
| 366132 | AA062805 | 6.22 | 1.68 | 2.75 | 3.72 | 2.26 | − | + | − | − |
| 701677 | AA287067 | 16.03 | 15.09 | 3.27 | 1.06 | 4.91 | − | − | + | + |
| 757462 | AA442301 | 12.77 | 6.52 | 3.19 | 1.96 | 4.01 | − | − | − | + |
| 1240220 | AA788970 | 7.60 | 2.71 | 2.40 | 2.80 | 3.17 | + | + | + | + |
| 210862 | H65659 | 2.56 | 1.16 | 0.68 | 2.21 | 3.76 | − | − | + | + |
| 530185 | AA083671 | 8.46 | 7.83 | 1.73 | 1.08 | 4.88 | − | − | + | + |
| 773248 | AA425773 | 21.90 | 19.94 | 4.51 | 1.10 | 4.86 | − | − | + | + |
| 772416 | AA405597 | 1.03 | 1.35 | 0.20 | 0.76 | 5.19 | − | − | + | + |
| 796513 | AA460251 | 17.83 | 7.09 | 5.20 | 2.51 | 3.43 | − | + | + | + |
| 66902 | T67444 | 3.21 | 1.14 | 1.03 | 2.82 | 3.12 | + | + | + | + |
| 825718 | AA505162 | 4.03 | 1.32 | 1.40 | 3.06 | 2.88 | − | + | − | + |
| 201282 | R99503 | 6.42 | 4.16 | 1.46 | 1.55 | 4.39 | − | − | + | + |
| 1604342 | AA987644 | 1.97 | 0.81 | 0.57 | 2.45 | 3.48 | + | − | + | + |
| 362926 | AA018979 | 4.39 | 2.04 | 1.16 | 2.15 | 3.77 | − | − | + | + |
| 1456721 | AA864875 | 12.82 | 5.49 | 3.58 | 2.33 | 3.58 | − | − | − | + |
| 1070015 | AA599717 | 3.26 | 1.43 | 0.90 | 2.28 | 3.63 | − | + | + | + |
| 109153 | T81140 | 5.46 | 1.70 | 2.03 | 3.20 | 2.69 | − | + | − | − |
| 50764 | H17363 | 0.43 | 0.23 | 0.11 | 1.89 | 4.00 | − | − | + | + |
| 356992 | W92964 | 21.95 | 7.93 | 7.04 | 2.77 | 3.12 | − | + | − | + |
| 1635874 | AA996104 | 6.06 | 2.17 | 1.97 | 2.79 | 3.07 | + | − | + | + |
| 824843 | AA488865 | 4.03 | 2.24 | 0.99 | 1.80 | 4.06 | − | − | + | + |
| 259462 | N29545 | 3.69 | 1.65 | 1.02 | 2.24 | 3.62 | + | − | + | − |
| 773208 | AA425205 | 26.40 | 17.78 | 6.05 | 1.49 | 4.37 | − | − | + | + |
| 626990 | AA190850 | 7.39 | 3.82 | 1.89 | 1.93 | 3.91 | − | − | + | + |
| 743749 | AA634291 | 5.10 | 1.65 | 1.86 | 3.10 | 2.74 | − | + | − | − |
| 79000 | T62164 | 6.24 | 3.53 | 1.54 | 1.77 | 4.05 | − | − | + | + |
| 23576 | T77385 | 3.58 | 1.59 | 1.00 | 2.25 | 3.57 | − | − | − | + |
| 366815 | AA029490 | 71.61 | 18.49 | 36.97 | 3.87 | 1.94 | + | − | + | − |
| 731016 | AA421256 | 0.85 | 1.00 | 0.17 | 0.85 | 4.96 | − | − | + | + |
| 415437 | W81124 | 3.52 | 1.02 | 1.50 | 3.46 | 2.35 | − | + | − | − |
| 197779 | R93515 | 8.61 | 6.61 | 1.91 | 1.30 | 4.50 | − | − | + | + |
| 1519515 | AA902831 | 0.85 | 0.55 | 0.20 | 1.55 | 4.25 | − | − | + | + |
| 511428 | AA126009 | 24.31 | 24.09 | 5.08 | 1.01 | 4.79 | − | − | + | + |
| 624785 | AA181995 | 6.17 | 2.49 | 1.86 | 2.47 | 3.32 | − | − | − | + |
| 809578 | AA456616 | 24.75 | 12.84 | 6.40 | 1.93 | 3.87 | − | − | + | + |
| 360403 | AA015658 | 28.16 | 15.79 | 7.03 | 1.78 | 4.01 | − | − | + | + |
| 431286 | AA682627 | 13.81 | 4.59 | 4.98 | 3.01 | 2.77 | − | + | − | + |
| 712216 | AA280288 | 0.50 | 0.30 | 0.12 | 1.68 | 4.10 | − | − | + | + |
| 296788 | W01113 | 3.27 | 0.67 | 3.50 | 4.85 | 0.93 | + | + | − | − |
| 1638852 | AI016618 | 1.21 | 0.50 | 0.36 | 2.41 | 3.36 | − | − | − | + |
| 1636868 | AI015589 | 5.07 | 2.89 | 1.26 | 1.76 | 4.01 | − | − | + | + |
| 461592 | AA705142 | 11.87 | 12.89 | 2.45 | 0.92 | 4.84 | − | − | + | + |
| 357364 | W93709 | 0.72 | 0.27 | 0.23 | 2.67 | 3.09 | − | − | + | + |
| 594517 | AA169645 | 24.81 | 12.50 | 6.57 | 1.98 | 3.78 | − | − | + | + |
| 842882 | AA486412 | 2.20 | 1.38 | 0.53 | 1.60 | 4.16 | − | − | + | + |
| 111812 | T84975 | 5.56 | 1.89 | 1.98 | 2.94 | 2.81 | − | + | − | + |
| 757206 | AA443969 | 5.25 | 4.09 | 1.17 | 1.28 | 4.47 | − | − | + | + |
| 292042 | W02333 | 9.39 | 2.67 | 4.19 | 3.51 | 2.24 | − | + | − | − |
| 743828 | AA634379 | 3.34 | 1.15 | 1.17 | 2.90 | 2.85 | + | + | + | + |
| 884657 | AA629910 | 6.68 | 2.72 | 2.04 | 2.46 | 3.28 | − | − | − | + |
| 743804 | AA634360 | 5.09 | 1.90 | 1.66 | 2.68 | 3.06 | − | − | − | + |
| 362829 | AA019445 | 3.03 | 1.24 | 0.92 | 2.44 | 3.30 | − | − | + | + |
| 502690 | AA127100 | 4.09 | 1.46 | 1.39 | 2.80 | 2.93 | − | + | − | + |
| 854678 | AA630084 | 6.21 | 2.55 | 1.89 | 2.44 | 3.29 | + | − | + | − |
| 626385 | AA189113 | 15.94 | 8.27 | 4.20 | 1.93 | 3.79 | − | − | + | + |
| 364022 | AA021586 | 4.15 | 1.53 | 1.39 | 2.72 | 3.00 | − | − | + | + |
| 264465 | N20522 | 0.77 | 0.84 | 0.16 | 0.91 | 4.79 | − | − | + | + |
| 232628 | H72591 | 4.02 | 1.27 | 1.58 | 3.16 | 2.55 | + | + | − | + |
| 950410 | AA599064 | 3.18 | 0.89 | 1.49 | 3.56 | 2.14 | + | − | − | − |
| 838761 | AA457767 | 2.59 | 2.11 | 0.58 | 1.23 | 4.47 | − | − | + | + |
| 753162 | AA400732 | 2.81 | 1.76 | 0.69 | 1.60 | 4.10 | − | − | + | + |
| 610011 | AA169192 | 34.63 | 11.27 | 13.23 | 3.07 | 2.62 | − | + | − | + |
| 251330 | H97880 | 9.21 | 3.78 | 2.83 | 2.44 | 3.25 | − | + | − | + |
| 823588 | AA497127 | 7.72 | 2.72 | 2.70 | 2.83 | 2.85 | − | + | − | + |
| 361048 | AA017383 | 3.13 | 1.69 | 0.82 | 1.85 | 3.83 | − | − | + | + |
| 826102 | AA521409 | 4.19 | 1.51 | 1.45 | 2.78 | 2.89 | + | − | + | − |
| 877832 | AA625628 | 44.43 | 13.65 | 18.48 | 3.25 | 2.40 | − | + | − | − |
| 856447 | AA630800 | 8.81 | 2.09 | 6.13 | 4.22 | 1.44 | + | + | − | − |
| 33715 | R44078 | 1.36 | 0.37 | 0.71 | 3.73 | 1.93 | − | + | − | − |
| 815549 | AA456827 | 2.30 | 1.97 | 0.51 | 1.17 | 4.49 | − | − | + | + |
| 768443 | AA495936 | 26.58 | 15.35 | 6.79 | 1.73 | 3.92 | − | − | + | + |
| 1456602 | AA864681 | 6.11 | 2.54 | 1.88 | 2.40 | 3.25 | − | − | − | + |
| 745103 | AA626365 | 8.87 | 3.33 | 2.98 | 2.67 | 2.97 | − | + | − | + |
| 784258 | AA447018 | 10.08 | 2.77 | 5.04 | 3.64 | 2.00 | + | + | − | − |
| 950836 | AA608679 | 34.79 | 13.60 | 11.31 | 2.56 | 3.07 | + | − | + | − |
| 609052 | AA176607 | 0.87 | 0.87 | 0.19 | 0.99 | 4.63 | − | − | + | + |
| 1637343 | AI015359 | 5.03 | 4.49 | 1.12 | 1.12 | 4.50 | − | − | + | + |
| 462325 | AA705516 | 8.76 | 3.83 | 2.63 | 2.29 | 3.33 | + | − | + | − |
| 878284 | AA670280 | 4.41 | 2.26 | 1.20 | 1.95 | 3.66 | − | − | + | + |
| 486538 | AA042812 | 7.64 | 5.06 | 1.86 | 1.51 | 4.10 | − | − | + | + |
| 878316 | AA670296 | 5.34 | 1.86 | 1.95 | 2.87 | 2.73 | − | + | − | + |
| 251019 | H97778 | 45.62 | 22.03 | 12.95 | 2.07 | 3.52 | − | − | + | + |
| 258167 | N40582 | 17.24 | 8.15 | 4.96 | 2.12 | 3.48 | − | − | + | + |
| 416833 | W87312 | 6.07 | 2.16 | 2.18 | 2.81 | 2.78 | − | + | − | + |
| 186768 | H51914 | 9.43 | 5.75 | 2.39 | 1.64 | 3.95 | − | − | + | − |
| 1558411 | AA976063 | 8.20 | 5.56 | 1.99 | 1.47 | 4.11 | − | − | + | + |
| 49728 | H29198 | 1.27 | 0.57 | 0.38 | 2.22 | 3.36 | − | − | − | + |
| 125722 | R07506 | 3.28 | 1.21 | 1.14 | 2.72 | 2.87 | − | + | − | + |
| 613237 | AA181767 | 1.07 | 0.78 | 0.25 | 1.37 | 4.22 | − | − | + | + |
| 526945 | AA112515 | 1.14 | 0.40 | 0.41 | 2.82 | 2.77 | − | + | − | + |
| 79032 | T61960 | 37.58 | 31.85 | 8.53 | 1.18 | 4.40 | − | − | + | + |
| 843139 | AA485922 | 39.96 | 18.13 | 11.84 | 2.20 | 3.37 | − | − | − | + |
| 841302 | AA487213 | 7.41 | 3.64 | 2.09 | 2.04 | 3.54 | + | − | + | − |
| 191978 | H38623 | 47.95 | 29.72 | 12.11 | 1.61 | 3.96 | − | − | + | + |
| 135900 | R33609 | 9.44 | 5.00 | 2.57 | 1.89 | 3.68 | − | − | + | + |
| 713109 | AA282983 | 2.44 | 0.98 | 0.79 | 2.48 | 3.08 | − | − | − | + |
| 626640 | AA191512 | 8.33 | 4.14 | 2.35 | 2.01 | 3.54 | − | − | + | + |
| 322914 | W45148 | 9.34 | 3.68 | 3.10 | 2.54 | 3.02 | − | − | − | + |
| 251517 | H96605 | 0.73 | 0.80 | 0.16 | 0.91 | 4.64 | − | − | + | + |
| 840878 | AA482228 | 25.81 | 27.85 | 5.59 | 0.93 | 4.61 | − | − | + | + |
| 626716 | AA191245 | 21.60 | 13.98 | 5.41 | 1.55 | 3.99 | − | − | + | + |
| 291345 | W02978 | 70.40 | 31.69 | 21.23 | 2.22 | 3.32 | + | − | + | + |
| 80500 | T64625 | 140.81 | 52.48 | 49.33 | 2.68 | 2.85 | − | + | + | + |
| 815737 | AA485201 | 3.64 | 2.20 | 0.94 | 1.65 | 3.88 | − | − | + | + |
| 436769 | AA703079 | 168.72 | 131.76 | 39.68 | 1.28 | 4.25 | − | − | + | + |
| 461509 | AA705077 | 0.62 | 0.38 | 0.16 | 1.66 | 3.87 | − | − | + | + |
| 427838 | AA001234 | 4.05 | 3.19 | 0.95 | 1.27 | 4.25 | − | − | + | + |
| 137638 | R37224 | 5.06 | 1.71 | 1.97 | 2.95 | 2.57 | − | + | − | + |
| 855624 | AA664101 | 17.75 | 8.17 | 5.31 | 2.17 | 3.34 | + | − | + | − |
| 109316 | T80924 | 12.35 | 4.01 | 5.08 | 3.08 | 2.43 | + | + | + | − |
| 725978 | AA399198 | 4.92 | 2.96 | 1.28 | 1.66 | 3.85 | − | − | + | + |
| 392678 | AA708342 | 34.95 | 10.61 | 15.79 | 3.29 | 2.21 | − | + | − | − |
| 120707 | T95670 | 19.60 | 12.65 | 4.95 | 1.55 | 3.96 | − | − | + | + |
| 841220 | AA486723 | 1.46 | 1.23 | 0.34 | 1.18 | 4.32 | − | − | − | + |
| 1631194 | AA994757 | 21.83 | 14.39 | 5.47 | 1.52 | 3.99 | − | − | + | + |
| 767798 | AA418755 | 5.70 | 1.98 | 2.18 | 2.88 | 2.61 | − | + | − | + |
| 251435 | H97989 | 0.73 | 0.90 | 0.16 | 0.81 | 4.68 | − | − | + | + |
| 75044 | T51904 | 3.46 | 0.93 | 1.96 | 3.72 | 1.77 | − | + | − | − |
| 743071 | AA405984 | 1.89 | 1.02 | 0.52 | 1.85 | 3.63 | − | − | + | + |
| 490766 | AA133165 | 3.48 | 1.03 | 1.66 | 3.38 | 2.10 | − | + | − | − |
| 344589 | W73144 | 53.28 | 21.92 | 17.55 | 2.43 | 3.04 | − | + | − | + |
| 250673 | H95978 | 2.66 | 1.12 | 0.86 | 2.37 | 3.09 | + | − | + | − |
| 785910 | AA449644 | 4.01 | 1.01 | 2.72 | 3.98 | 1.48 | + | + | − | − |
| 824329 | AA489670 | 1.94 | 1.47 | 0.47 | 1.32 | 4.13 | − | − | + | + |
| 1420790 | AA826237 | 13.40 | 6.84 | 3.84 | 1.96 | 3.49 | − | − | − | + |
| 430318 | AA010608 | 10.87 | 4.02 | 3.96 | 2.71 | 2.74 | − | + | − | + |
| 45564 | H05264 | 1.66 | 0.76 | 0.51 | 2.17 | 3.28 | − | − | + | + |
| 251936 | H97488 | 7.48 | 2.74 | 2.76 | 2.73 | 2.71 | − | + | − | − |
| 253314 | H89330 | 1.52 | 1.34 | 0.35 | 1.14 | 4.30 | − | − | + | + |
| 490232 | AA121428 | 2.68 | 0.85 | 1.17 | 3.15 | 2.28 | − | + | − | − |
| 855244 | AA630545 | 15.78 | 6.78 | 5.09 | 2.33 | 3.10 | + | + | + | + |
| 898242 | AA598621 | 10.34 | 7.74 | 2.53 | 1.34 | 4.09 | − | − | + | + |
| 395625 | AA757604 | 14.45 | 6.11 | 4.73 | 2.37 | 3.06 | − | − | − | + |
| 767406 | AA417911 | 0.44 | 0.45 | 0.10 | 0.98 | 4.45 | − | − | + | + |
| 264640 | N27610 | 13.22 | 6.10 | 4.07 | 2.17 | 3.25 | − | − | + | + |
| 1031598 | AA609474 | 5.18 | 1.34 | 3.37 | 3.88 | 1.54 | + | + | − | − |
| 785913 | AA449657 | 3.35 | 0.80 | 2.75 | 4.19 | 1.22 | + | + | − | − |
| 343987 | W70234 | 11.55 | 12.13 | 2.59 | 0.95 | 4.46 | − | − | + | + |
| 141505 | R72977 | 6.38 | 1.86 | 3.21 | 3.42 | 1.99 | − | + | − | − |
| 298155 | N70794 | 2.85 | 1.29 | 0.89 | 2.21 | 3.19 | − | − | − | + |
| 745283 | AA625567 | 7.94 | 5.76 | 1.97 | 1.38 | 4.02 | − | − | − | + |
| 26230 | R12449 | 5.08 | 2.29 | 1.60 | 2.22 | 3.18 | − | − | − | + |
| 743851 | AA634424 | 8.22 | 3.22 | 2.89 | 2.55 | 2.84 | − | + | − | + |
| 884531 | AA629801 | 7.71 | 4.73 | 2.05 | 1.63 | 3.76 | − | − | + | + |
| 345032 | W76319 | 20.78 | 9.11 | 6.69 | 2.28 | 3.11 | − | − | − | + |
| 729953 | AA412049 | 4.95 | 3.66 | 1.23 | 1.35 | 4.03 | − | − | + | + |
| 815017 | AA465258 | 14.81 | 9.71 | 3.84 | 1.52 | 3.85 | − | − | − | + |
| 308466 | N95495 | 9.26 | 4.85 | 2.67 | 1.91 | 3.47 | − | − | − | + |
| 773345 | AA425442 | 1.21 | 1.11 | 0.28 | 1.09 | 4.29 | − | − | + | + |
| 811766 | AA443004 | 4.93 | 3.60 | 1.23 | 1.37 | 4.00 | − | − | + | + |
| 246869 | N59119 | 4.68 | 1.38 | 2.37 | 3.40 | 1.97 | − | + | − | − |
| 1473300 | AA916323 | 12.44 | 4.26 | 5.09 | 2.92 | 2.44 | + | + | − | − |
| 447518 | AA702247 | 10.98 | 6.11 | 3.08 | 1.80 | 3.56 | − | − | − | + |
| 784093 | AA446643 | 6.44 | 3.83 | 1.75 | 1.68 | 3.68 | − | − | − | + |
| 293243 | N68686 | 13.17 | 5.84 | 4.25 | 2.26 | 3.10 | − | + | + | + |
| 897733 | AA598996 | 5.86 | 3.56 | 1.58 | 1.65 | 3.71 | − | − | − | + |
| 755409 | AA424706 | 7.81 | 2.43 | 3.67 | 3.22 | 2.13 | − | + | − | − |
| 299723 | N75055 | 2.98 | 2.09 | 0.76 | 1.43 | 3.93 | − | − | + | + |
| 461499 | AA705072 | 2.26 | 2.09 | 0.53 | 1.08 | 4.27 | − | − | + | + |
| 82905 | T69305 | 1.21 | 0.48 | 0.43 | 2.50 | 2.85 | − | + | − | + |
| 824917 | AA489121 | 1.74 | 1.05 | 0.47 | 1.65 | 3.69 | − | − | − | + |
| 121436 | T97457 | 6.37 | 6.46 | 1.46 | 0.99 | 4.35 | − | − | + | + |
| 950430 | AA599078 | 7.48 | 3.82 | 2.21 | 1.96 | 3.38 | − | − | − | + |
| 809657 | AA456331 | 4.72 | 2.68 | 1.32 | 1.76 | 3.57 | − | − | + | + |
| 32509 | R43053 | 4.38 | 3.20 | 1.10 | 1.37 | 3.97 | − | − | + | + |
| 346889 | W79834 | 0.89 | 0.21 | 0.79 | 4.21 | 1.12 | + | + | − | − |
| 145388 | R78386 | 2.66 | 0.94 | 1.06 | 2.83 | 2.50 | − | + | − | − |
| 431296 | AA682631 | 1.85 | 1.07 | 0.51 | 1.73 | 3.59 | − | − | + | + |
| 46411 | H09222 | 3.07 | 2.00 | 0.81 | 1.54 | 3.78 | − | − | + | + |
| 726483 | AA399269 | 3.50 | 2.68 | 0.87 | 1.30 | 4.01 | − | − | + | + |
| 823895 | AA490490 | 1.57 | 1.72 | 0.36 | 0.91 | 4.40 | − | − | − | + |
| 288695 | N59206 | 3.41 | 0.88 | 2.36 | 3.86 | 1.44 | + | + | − | − |
| 753092 | AA436595 | 7.06 | 3.62 | 2.10 | 1.95 | 3.36 | − | − | − | + |
| 845345 | AA773478 | 5.09 | 3.00 | 1.41 | 1.70 | 3.61 | − | − | − | + |
| 133130 | R28397 | 11.12 | 4.91 | 3.67 | 2.27 | 3.03 | − | − | + | + |
| 788541 | AA452955 | 12.11 | 3.01 | 9.65 | 4.03 | 1.26 | + | + | − | − |
| 417867 | W90224 | 13.52 | 9.26 | 3.54 | 1.46 | 3.82 | − | − | − | + |
| 611150 | AA173109 | 7.99 | 6.62 | 1.96 | 1.21 | 4.07 | − | − | + | + |
| 1553306 | AA934762 | 7.92 | 5.52 | 2.06 | 1.43 | 3.84 | − | − | − | + |
| 358456 | W96107 | 37.98 | 23.51 | 10.38 | 1.62 | 3.66 | − | − | − | + |
| 810510 | AA464531 | 27.71 | 20.86 | 7.03 | 1.33 | 3.94 | − | − | − | + |
| 361922 | AA001403 | 9.18 | 5.49 | 2.55 | 1.67 | 3.59 | − | − | − | + |
| 51921 | H22928 | 4.48 | 1.72 | 1.68 | 2.60 | 2.67 | − | − | + | − |
| 767994 | AA418821 | 4.29 | 1.83 | 1.47 | 2.34 | 2.93 | + | − | + | − |
| 754192 | AA479133 | 3.30 | 1.49 | 1.08 | 2.22 | 3.04 | − | − | − | + |
| 254310 | N81158 | 10.26 | 4.39 | 3.51 | 2.33 | 2.92 | + | − | + | + |
| 1635110 | AA994811 | 38.13 | 17.09 | 12.62 | 2.23 | 3.02 | − | − | − | + |
| 121997 | T98253 | 5.10 | 1.52 | 2.70 | 3.36 | 1.89 | + | + | − | − |
| 51011 | H19242 | 4.64 | 2.46 | 1.38 | 1.88 | 3.36 | − | − | + | + |
| 283034 | NS1280 | 9.61 | 3.05 | 4.60 | 3.15 | 2.09 | + | + | − | − |
| 564537 | AA121732 | 1.79 | 1.93 | 0.42 | 0.93 | 4.31 | − | − | + | + |
| 82734 | T73651 | 7.21 | 6.90 | 1.72 | 1.05 | 4.18 | − | − | + | + |
| 1631682 | AA994801 | 1.61 | 0.53 | 0.74 | 3.03 | 2.19 | − | + | − | − |
| 290378 | N64508 | 17.10 | 17.02 | 4.07 | 1.00 | 4.20 | − | − | + | + |
| 262865 | H99650 | 30.48 | 11.78 | 11.64 | 2.59 | 2.62 | − | + | − | + |
| 1031698 | AA609556 | 6.26 | 3.13 | 1.95 | 2.00 | 3.21 | − | − | − | + |
| 109200 | T81107 | 3.17 | 0.93 | 1.78 | 3.42 | 1.79 | + | + | − | − |
| 824358 | AA489681 | 0.96 | 0.68 | 0.25 | 1.42 | 3.78 | − | − | + | + |
| 32684 | R20424 | 24.32 | 17.98 | 6.32 | 1.35 | 3.85 | − | − | + | + |
| 240430 | H78097 | 3.52 | 1.28 | 1.43 | 2.74 | 2.46 | + | − | − | − |
| 1475200 | AA858390 | 28.16 | 22.56 | 7.13 | 1.25 | 3.95 | − | − | + | + |
| 731272 | AA421073 | 5.02 | 2.47 | 1.59 | 2.03 | 3.16 | − | − | − | + |
| 245860 | N55339 | 30.36 | 12.47 | 11.02 | 2.43 | 2.76 | − | + | + | + |
| 854874 | AA630346 | 2.22 | 0.68 | 1.15 | 3.26 | 1.92 | + | + | − | − |
| 824124 | AA490802 | 1.03 | 0.63 | 0.29 | 1.63 | 3.55 | − | − | − | + |
| 1435029 | AA857212 | 1.84 | 0.67 | 0.75 | 2.74 | 2.44 | − | + | − | − |
| 951080 | AA620446 | 2.43 | 1.63 | 0.66 | 1.49 | 3.69 | − | − | + | + |
| 22731 | R45264 | 4.46 | 2.73 | 1.26 | 1.64 | 3.54 | − | − | + | + |
| 813637 | AA447742 | 34.08 | 17.95 | 10.41 | 1.90 | 3.27 | − | − | + | + |
| 258521 | N40743 | 3.33 | 1.39 | 1.20 | 2.40 | 2.77 | + | − | + | − |
| 1602619 | AA988798 | 135.32 | 117.37 | 33.70 | 1.15 | 4.01 | − | − | − | + |
| 144926 | R78559 | 9.20 | 3.96 | 3.24 | 2.32 | 2.84 | − | + | − | + |
| 897690 | AA598758 | 15.87 | 13.03 | 4.03 | 1.22 | 3.94 | − | − | + | + |
| 740914 | AA478268 | 7.20 | 4.16 | 2.10 | 1.73 | 3.42 | − | − | + | + |
| 1160558 | AA877347 | 8.03 | 3.31 | 2.94 | 2.43 | 2.73 | − | − | − | + |
| 277660 | N49405 | 14.16 | 5.37 | 5.65 | 2.64 | 2.51 | − | + | − | + |
| 840821 | AA486261 | 17.59 | 8.78 | 5.60 | 2.00 | 3.14 | − | − | − | + |
| 760344 | AA426227 | 5.89 | 3.00 | 1.85 | 1.96 | 3.18 | − | − | − | + |
| 813187 | AA456721 | 22.70 | 10.74 | 7.51 | 2.11 | 3.02 | − | − | − | + |
| 685516 | AA262573 | 3.36 | 1.79 | 1.03 | 1.88 | 3.26 | − | − | − | + |
| 503851 | AA130042 | 18.15 | 5.54 | 9.79 | 3.28 | 1.85 | + | + | − | − |
| 1631762 | AI025126 | 46.32 | 36.08 | 12.06 | 1.28 | 3.84 | − | − | − | + |
| 343167 | W67309 | 15.62 | 7.28 | 5.24 | 2.15 | 2.98 | − | − | − | + |
| 30793 | R18258 | 7.89 | 2.84 | 3.37 | 2.78 | 2.34 | − | + | − | − |
| 82874 | T69348 | 5.14 | 2.70 | 1.60 | 1.90 | 3.21 | − | − | − | + |
| 209167 | H63668 | 3.29 | 1.15 | 1.46 | 2.86 | 2.25 | + | + | + | − |
| 811842 | AA443177 | 9.91 | 4.54 | 3.39 | 2.18 | 2.92 | − | − | − | + |
| 810221 | AA464700 | 3.85 | 1.37 | 1.68 | 2.81 | 2.30 | − | + | − | − |
| 249705 | H85464 | 62.65 | 37.20 | 18.34 | 1.68 | 3.42 | − | − | − | + |
| 178137 | H47015 | 238.41 | 190.87 | 61.93 | 1.25 | 3.85 | − | − | + | + |
| 815800 | AA485216 | 1.22 | 1.44 | 0.29 | 0.84 | 4.25 | − | − | + | + |
| 294259 | W01800 | 4.21 | 2.21 | 1.32 | 1.90 | 3.19 | − | − | + | + |
| 248454 | N59626 | 3.70 | 1.95 | 1.16 | 1.89 | 3.19 | − | − | + | − |
| 504290 | AA151796 | 50.88 | 17.89 | 22.72 | 2.84 | 2.24 | + | − | + | − |
| 731257 | AA416833 | 4.45 | 2.31 | 1.41 | 1.93 | 3.15 | − | − | + | + |
| 259350 | N41826 | 22.84 | 6.64 | 13.93 | 3.44 | 1.64 | − | + | − | − |
| 1493390 | AA894687 | 3.57 | 1.31 | 1.53 | 2.73 | 2.34 | − | + | − | + |
| 665127 | AA195601 | 5.48 | 2.72 | 1.79 | 2.02 | 3.06 | − | − | − | + |
| 377672 | AA056001 | 2.59 | 1.39 | 0.81 | 1.87 | 3.20 | + | − | + | − |
| 837953 | AA458578 | 10.44 | 6.85 | 2.94 | 1.52 | 3.55 | − | − | + | + |
| 1500324 | AA885819 | 6.77 | 4.55 | 1.89 | 1.49 | 3.58 | − | − | + | + |
| 167205 | R90934 | 7.85 | 2.56 | 3.93 | 3.06 | 2.00 | + | + | − | − |
| 594693 | AA165313 | 7.33 | 3.57 | 2.44 | 2.05 | 3.00 | − | − | + | + |
| 868119 | AA634213 | 10.00 | 4.58 | 3.49 | 2.18 | 2.87 | − | − | − | + |
| 502463 | AA134770 | 5.77 | 2.99 | 1.85 | 1.93 | 3.12 | − | − | − | + |
| 342271 | W61215 | 3.90 | 1.09 | 2.69 | 3.60 | 1.45 | + | + | − | − |
| 256680 | H96392 | 1.72 | 1.47 | 0.44 | 1.17 | 3.88 | − | − | − | + |
| 430179 | AA010091 | 51.95 | 32.22 | 15.13 | 1.61 | 3.43 | − | − | + | − |
| 897153 | AA676961 | 10.77 | 5.36 | 3.55 | 2.01 | 3.04 | − | − | − | + |
| 25517 | R11888 | 8.52 | 4.10 | 2.87 | 2.08 | 2.96 | − | − | − | + |
| 1632141 | AI004916 | 2.15 | 0.93 | 0.79 | 2.31 | 2.74 | − | − | − | + |
| 814018 | AA455714 | 0.98 | 0.77 | 0.26 | 1.28 | 3.77 | − | − | + | + |
| 843234 | AA488588 | 7.02 | 2.32 | 3.49 | 3.03 | 2.01 | − | + | − | − |
| 823578 | AA497118 | 0.56 | 0.53 | 0.14 | 1.06 | 3.98 | − | − | − | + |
| 42636 | R61780 | 1.11 | 0.32 | 0.72 | 3.50 | 1.54 | − | + | − | − |
| 784830 | AA448289 | 1.83 | 0.63 | 0.85 | 2.88 | 2.15 | − | + | − | − |
| 244652 | N52911 | 24.70 | 12.93 | 7.92 | 1.91 | 3.12 | − | − | + | − |
| 950709 | AA608583 | 4.17 | 1.22 | 2.62 | 3.43 | 1.59 | + | + | − | − |
| 1486260 | AA922326 | 13.23 | 7.43 | 4.08 | 1.78 | 3.24 | − | − | + | + |
| 878152 | AA775431 | 4.20 | 3.49 | 1.10 | 1.20 | 3.82 | − | − | − | + |
| 130773 | R22156 | 3.71 | 1.21 | 1.90 | 3.07 | 1.95 | − | + | − | − |
| 586725 | AA129171 | 2.32 | 0.70 | 1.34 | 3.29 | 1.73 | − | + | − | − |
| 511521 | AA126356 | 33.96 | 23.00 | 9.58 | 1.48 | 3.54 | − | − | − | + |
| 295206 | N75947 | 3.16 | 1.64 | 1.02 | 1.92 | 3.10 | − | − | + | + |
| 281072 | N50912 | 5.02 | 1.92 | 2.10 | 2.62 | 2.39 | + | − | − | − |
| 824704 | AA482198 | 2.04 | 0.70 | 0.98 | 2.91 | 2.09 | − | + | − | − |
| 143306 | R74169 | 10.42 | 4.91 | 3.62 | 2.12 | 2.88 | − | − | + | + |
| 247609 | N58148 | 3.15 | 1.20 | 1.33 | 2.63 | 2.37 | − | + | − | − |
| 250678 | H95976 | 4.24 | 1.94 | 1.51 | 2.18 | 2.82 | − | − | + | + |
| 814427 | AA458943 | 0.47 | 0.34 | 0.13 | 1.38 | 3.62 | − | − | + | + |
| 825058 | AA504284 | 3.42 | 1.55 | 1.23 | 2.21 | 2.79 | + | − | − | − |
| 197888 | R96220 | 4.97 | 4.63 | 1.27 | 1.07 | 3.92 | − | − | + | + |
| 261472 | H99035 | 9.31 | 3.39 | 4.15 | 2.75 | 2.25 | + | − | − | − |
| 137254 | R36523 | 1.35 | 0.58 | 0.51 | 2.35 | 2.64 | − | + | − | + |
| 52730 | H29499 | 6.16 | 4.38 | 1.72 | 1.41 | 3.58 | − | − | + | + |
| 76252 | T59873 | 11.64 | 8.74 | 3.19 | 1.33 | 3.65 | − | − | + | + |
| 898123 | AA598487 | 4.77 | 1.81 | 2.03 | 2.63 | 2.35 | − | + | − | − |
| 815794 | AA485214 | 14.69 | 7.77 | 4.77 | 1.89 | 3.08 | − | − | + | + |
| 685922 | AA262559 | 3.59 | 3.70 | 0.90 | 0.97 | 3.99 | − | − | + | + |
| 233464 | H77297 | 14.24 | 3.73 | 12.38 | 3.81 | 1.15 | + | + | − | − |
| 80484 | T64609 | 14.69 | 14.52 | 3.72 | 1.01 | 3.95 | − | − | + | + |
| 322051 | W37306 | 11.58 | 4.27 | 5.15 | 2.71 | 2.25 | − | + | − | − |
| 299154 | W05406 | 1.37 | 0.71 | 0.45 | 1.92 | 3.04 | − | − | − | + |
| 136605 | R35051 | 10.58 | 5.59 | 3.46 | 1.89 | 3.06 | − | − | + | + |
| 712570 | AA281504 | 1.32 | 1.16 | 0.35 | 1.14 | 3.81 | − | − | − | + |
| 855634 | AA664105 | 3.16 | 1.34 | 1.22 | 2.36 | 2.59 | − | − | + | + |
| 272576 | N43976 | 6.22 | 4.17 | 1.80 | 1.49 | 3.45 | − | − | − | + |
| 83363 | T68453 | 5.08 | 3.35 | 1.48 | 1.51 | 3.43 | − | − | − | + |
| 1536991 | AA933888 | 10.79 | 3.85 | 5.05 | 2.80 | 2.14 | + | + | − | + |
| 755750 | AA496628 | 5.88 | 4.08 | 1.68 | 1.44 | 3.50 | − | − | + | + |
| 795555 | AA459679 | 3.38 | 1.25 | 1.51 | 2.70 | 2.24 | − | + | − | − |
| 148225 | H13738 | 3.89 | 1.96 | 1.31 | 1.98 | 2.96 | − | − | + | − |
| 188151 | H45810 | 3.25 | 1.41 | 1.23 | 2.30 | 2.64 | − | − | + | − |
| 786609 | AA478481 | 4.81 | 2.23 | 1.73 | 2.15 | 2.78 | + | − | + | − |
| 561971 | AA088886 | 3.81 | 3.31 | 1.01 | 1.15 | 3.78 | − | − | − | + |
| 950603 | AA608546 | 3.53 | 2.28 | 1.04 | 1.55 | 3.39 | − | − | + | − |
| 266747 | N31312 | 3.05 | 1.60 | 1.01 | 1.91 | 3.02 | − | − | − | + |
| 129146 | R10947 | 7.97 | 4.87 | 2.42 | 1.64 | 3.30 | − | − | − | + |
| 41940 | R59087 | 10.51 | 5.11 | 3.65 | 2.05 | 2.88 | − | − | − | + |
| 786525 | AA452113 | 1.46 | 0.42 | 1.00 | 3.46 | 1.46 | + | − | − | − |
| 530814 | AA070226 | 46.55 | 26.81 | 14.60 | 1.74 | 3.19 | − | − | − | + |
| 287528 | N62096 | 1.98 | 1.06 | 0.65 | 1.87 | 3.05 | − | − | + | + |
| 701112 | AA287323 | 8.70 | 5.76 | 2.55 | 1.51 | 3.41 | − | − | − | + |
| 1435185 | AA857632 | 2.56 | 1.69 | 0.75 | 1.51 | 3.40 | − | − | + | + |
| 248261 | N78083 | 1.51 | 0.70 | 0.55 | 2.15 | 2.76 | − | + | + | + |
| 951091 | AA620458 | 2.03 | 1.20 | 0.63 | 1.69 | 3.22 | − | − | − | + |
| 123755 | R01304 | 5.06 | 2.03 | 2.10 | 2.49 | 2.41 | + | + | + | + |
| 505344 | AA147499 | 2.26 | 1.01 | 0.84 | 2.23 | 2.68 | − | − | − | + |
| 827152 | AA521247 | 3.50 | 2.11 | 1.08 | 1.66 | 3.24 | − | − | + | + |
| 812977 | AA464617 | 7.62 | 5.19 | 2.22 | 1.47 | 3.43 | − | − | + | + |
| 1521977 | AA906997 | 2.08 | 0.72 | 1.04 | 2.90 | 2.00 | − | + | − | − |
| 416010 | W85832 | 53.71 | 32.20 | 16.66 | 1.67 | 3.22 | − | − | + | − |
| 1031086 | AA610071 | 25.15 | 13.97 | 8.15 | 1.80 | 3.09 | − | − | + | + |
| 878525 | AA775857 | 11.10 | 9.66 | 2.97 | 1.15 | 3.74 | − | − | − | + |
| 430153 | AA010233 | 4.46 | 1.54 | 2.25 | 2.90 | 1.98 | − | + | − | − |
| 417801 | W88848 | 17.18 | 13.47 | 4.77 | 1.28 | 3.60 | − | − | + | + |
| 108265 | T70810 | 7.24 | 5.07 | 2.10 | 1.43 | 3.45 | − | − | − | + |
| 745296 | AA625574 | 29.18 | 19.75 | 8.59 | 1.48 | 3.40 | − | − | + | + |
| 365098 | AA025112 | 4.93 | 1.78 | 2.34 | 2.76 | 2.11 | − | + | − | − |
| 112785 | T85958 | 7.75 | 4.81 | 2.38 | 1.61 | 3.25 | − | − | − | + |
| 813651 | AA447761 | 4.96 | 1.93 | 2.17 | 2.58 | 2.29 | − | + | − | − |
| 810284 | AA463932 | 2.27 | 1.27 | 0.74 | 1.79 | 3.07 | − | − | + | + |
| 726488 | AA399264 | 10.10 | 6.03 | 3.17 | 1.68 | 3.18 | − | − | − | + |
| 512116 | AA133721 | 4.59 | 1.55 | 2.41 | 2.96 | 1.90 | + | − | + | − |
| 815760 | AA485018 | 2.31 | 1.17 | 0.81 | 1.98 | 2.87 | − | − | − | + |
| 50344 | H17002 | 0.85 | 0.39 | 0.32 | 2.16 | 2.69 | + | − | + | − |
| 293830 | N98283 | 4.43 | 1.72 | 1.96 | 2.58 | 2.26 | + | − | + | − |
| 213890 | H72937 | 14.14 | 7.99 | 4.60 | 1.77 | 3.07 | − | − | + | + |
| 784225 | AA446885 | 10.62 | 7.56 | 3.09 | 1.41 | 3.44 | − | − | − | + |
| 36607 | R46837 | 7.52 | 6.30 | 2.06 | 1.19 | 3.65 | − | − | + | + |
| 703707 | AA278195 | 4.12 | 2.45 | 1.31 | 1.68 | 3.15 | − | − | − | + |
| 40017 | R53311 | 8.29 | 4.13 | 2.94 | 2.01 | 2.82 | − | − | − | + |
| 669371 | AA253444 | 4.99 | 2.55 | 1.74 | 1.96 | 2.87 | − | − | + | − |
| 490360 | AA122048 | 5.94 | 3.29 | 1.97 | 1.81 | 3.02 | − | − | − | + |
| 544639 | AA074677 | 2.82 | 1.94 | 0.84 | 1.46 | 3.37 | − | − | − | + |
| 489109 | AA056484 | 7.44 | 5.33 | 2.17 | 1.40 | 3.43 | − | − | + | + |
| 279394 | N48701 | 3.18 | 1.23 | 1.42 | 2.59 | 2.23 | + | − | + | − |
| 74223 | T48411 | 5.42 | 3.10 | 1.76 | 1.75 | 3.07 | − | − | − | + |
| 381036 | AA054643 | 3.54 | 1.49 | 1.46 | 2.38 | 2.43 | + | − | + | − |
| 712610 | AA281793 | 1.06 | 0.58 | 0.35 | 1.83 | 2.98 | − | − | − | + |
| 448117 | AA702480 | 3.91 | 1.64 | 1.61 | 2.38 | 2.43 | − | + | − | + |
| 50114 | H16851 | 0.83 | 0.28 | 0.44 | 2.92 | 1.89 | + | + | − | + |
| 1472638 | AA872323 | 7.11 | 4.65 | 2.16 | 1.53 | 3.29 | − | − | − | + |
| 841399 | AA487552 | 9.08 | 4.37 | 3.33 | 2.08 | 2.73 | − | − | − | + |
| 855799 | AA664067 | 9.00 | 6.46 | 2.64 | 1.39 | 3.41 | − | − | + | + |
| 810753 | AA457737 | 4.95 | 1.68 | 2.67 | 2.95 | 1.85 | − | + | − | − |
| 51749 | H23049 | 3.65 | 1.79 | 1.33 | 2.05 | 2.75 | − | − | + | − |
| 361659 | W96280 | 13.43 | 4.23 | 8.34 | 3.18 | 1.61 | − | + | − | − |
| 252453 | H87143 | 20.12 | 14.53 | 5.91 | 1.38 | 3.40 | − | − | + | + |
| 133717 | R27767 | 9.46 | 2.97 | 5.91 | 3.18 | 1.60 | + | + | − | − |
| 785793 | AA449037 | 55.68 | 21.05 | 26.01 | 2.65 | 2.14 | − | + | − | − |
| 76196 | T59668 | 2.53 | 0.90 | 1.29 | 2.83 | 1.96 | + | + | − | − |
| 322561 | W15277 | 107.98 | 70.00 | 33.31 | 1.54 | 3.24 | − | − | − | + |
| 825833 | AA505150 | 1.95 | 1.08 | 0.66 | 1.81 | 2.97 | − | − | − | + |
| 755765 | AA496455 | 4.01 | 2.20 | 1.36 | 1.83 | 2.96 | − | − | − | + |
| 1416086 | AA878210 | 8.24 | 4.30 | 2.88 | 1.92 | 2.86 | − | − | + | + |
| 358083 | W95279 | 2.45 | 0.80 | 1.44 | 3.07 | 1.71 | − | + | − | − |
| 813636 | AA447731 | 84.51 | 49.31 | 27.59 | 1.71 | 3.06 | − | − | − | + |
| 490753 | AA133166 | 5.35 | 3.78 | 1.60 | 1.42 | 3.35 | − | − | + | + |
| 809876 | AA455126 | 27.74 | 19.39 | 8.32 | 1.43 | 3.34 | − | − | − | + |
| 744933 | AA625812 | 2.15 | 1.01 | 0.82 | 2.14 | 2.63 | + | − | + | − |
| 321386 | W44852 | 2.45 | 1.56 | 0.77 | 1.57 | 3.19 | − | − | − | + |
| 253577 | H89589 | 1.48 | 1.24 | 0.41 | 1.19 | 3.57 | − | − | − | + |
| 41174 | R56106 | 4.02 | 2.38 | 1.31 | 1.69 | 3.07 | − | − | − | + |
| 149013 | R82299 | 7.82 | 5.75 | 2.30 | 1.36 | 3.40 | − | − | + | + |
| 296529 | W01011 | 9.83 | 7.09 | 2.92 | 1.39 | 3.37 | − | − | − | + |
| 361097 | AA017200 | 5.63 | 3.33 | 1.84 | 1.69 | 3.06 | − | − | − | + |
| 154749 | R55619 | 1.76 | 1.07 | 0.57 | 1.65 | 3.10 | − | − | − | + |
| 197176 | R92806 | 20.79 | 10.24 | 7.65 | 2.03 | 2.72 | − | − | − | + |
| 627428 | AA190313 | 24.22 | 21.29 | 6.72 | 1.14 | 3.61 | − | − | + | + |
| 898198 | AA598561 | 14.33 | 5.93 | 6.17 | 2.42 | 2.32 | − | + | − | − |
| 590298 | AA147837 | 10.43 | 6.02 | 3.47 | 1.73 | 3.00 | − | − | − | + |
| 486567 | AA042928 | 9.14 | 5.47 | 2.99 | 1.67 | 3.06 | − | − | − | + |
| 1475797 | AA872122 | 85.21 | 65.01 | 24.92 | 1.31 | 3.42 | − | − | + | − |
| 283196 | N54728 | 3.80 | 2.01 | 1.34 | 1.89 | 2.84 | − | − | + | − |
| 502622 | AA126708 | 18.65 | 13.11 | 5.65 | 1.42 | 3.30 | − | − | + | + |
| 1476065 | AA873060 | 6.33 | 4.83 | 1.85 | 1.31 | 3.41 | − | − | + | + |
| 754221 | AA479154 | 6.80 | 4.46 | 2.13 | 1.53 | 3.20 | − | − | + | + |
| 325606 | W52341 | 7.30 | 3.27 | 2.93 | 2.23 | 2.49 | − | − | − | + |
| 813249 | AA455917 | 14.02 | 6.63 | 5.40 | 2.11 | 2.60 | − | − | − | + |
| 32076 | R17320 | 1.03 | 0.37 | 0.54 | 2.79 | 1.92 | − | + | − | − |
| 448930 | AA777781 | 7.53 | 4.62 | 2.45 | 1.63 | 3.08 | − | − | − | + |
| 1602120 | AA962236 | 6.44 | 4.29 | 2.01 | 1.50 | 3.21 | − | − | + | + |
| 855487 | AA664155 | 8.56 | 4.10 | 3.27 | 2.09 | 2.62 | − | − | − | + |
| 454672 | AA677185 | 7.68 | 5.14 | 2.40 | 1.49 | 3.20 | − | − | + | + |
| 26616 | R13557 | 1.94 | 0.59 | 1.40 | 3.31 | 1.39 | − | + | − | − |
| 1594019 | AA988701 | 24.27 | 24.53 | 6.55 | 0.99 | 3.70 | − | − | − | + |
| 342721 | W68291 | 14.93 | 6.70 | 6.06 | 2.23 | 2.46 | − | − | + | + |
| 590145 | AA156202 | 4.37 | 5.11 | 1.14 | 0.85 | 3.84 | − | − | + | + |
| 843150 | AA488481 | 5.13 | 1.90 | 2.57 | 2.69 | 1.99 | − | + | − | − |
| 282283 | N52675 | 0.84 | 1.00 | 0.22 | 0.84 | 3.85 | − | − | − | + |
| 203132 | H54628 | 12.63 | 11.94 | 3.48 | 1.06 | 3.63 | − | − | + | + |
| 40338 | R53072 | 2.61 | 1.86 | 0.79 | 1.40 | 3.28 | − | − | − | + |
| 204146 | H55920 | 2.40 | 1.26 | 0.86 | 1.90 | 2.78 | − | − | + | − |
| 782383 | AA431407 | 1.32 | 0.85 | 0.42 | 1.55 | 3.13 | − | − | + | − |
| 141972 | R68909 | 18.88 | 10.40 | 6.60 | 1.82 | 2.86 | − | − | − | + |
| 356863 | W84627 | 3.18 | 0.94 | 2.45 | 3.38 | 1.30 | − | + | − | − |
| 471266 | AA033564 | 2.72 | 1.68 | 0.89 | 1.62 | 3.05 | − | − | − | + |
| 246620 | N53133 | 16.85 | 15.47 | 4.71 | 1.09 | 3.58 | − | − | − | + |
| 811121 | AA485688 | 1.07 | 1.34 | 0.28 | 0.80 | 3.87 | − | − | − | + |
| 701532 | AA284337 | 3.26 | 2.02 | 1.07 | 1.61 | 3.05 | − | − | − | + |
| 42659 | R61126 | 10.21 | 3.36 | 6.30 | 3.04 | 1.62 | − | + | − | − |
| 460841 | AA708189 | 1.02 | 0.58 | 0.35 | 1.75 | 2.91 | − | − | − | + |
| 306771 | N91887 | 3.61 | 1.62 | 1.49 | 2.23 | 2.42 | − | − | − | + |
| 160485 | H22136 | 5.17 | 2.96 | 1.78 | 1.74 | 2.90 | − | − | + | + |
| 950461 | AA599107 | 38.86 | 34.73 | 11.02 | 1.12 | 3.53 | − | − | + | + |
| 75886 | T59478 | 14.08 | 9.92 | 4.36 | 1.42 | 3.23 | − | − | + | + |
| 283919 | N50797 | 7.56 | 4.94 | 2.43 | 1.53 | 3.11 | − | − | + | + |
| 813271 | AA455938 | 0.96 | 1.12 | 0.25 | 0.85 | 3.79 | − | − | − | + |
| 823627 | AA490561 | 3.25 | 1.81 | 1.14 | 1.79 | 2.85 | − | − | − | + |
| 242706 | H93552 | 5.15 | 2.96 | 1.78 | 1.74 | 2.90 | − | − | − | + |
| 970271 | AA775957 | 3.16 | 1.23 | 1.52 | 2.56 | 2.07 | − | + | − | − |
| 783645 | AA446690 | 6.96 | 2.63 | 3.51 | 2.65 | 1.98 | − | + | − | − |
| 486459 | AA042858 | 6.24 | 5.89 | 1.75 | 1.06 | 3.57 | − | − | + | + |
| 884346 | AA629524 | 5.38 | 3.61 | 1.71 | 1.49 | 3.14 | − | − | − | + |
| 26406 | R20638 | 6.56 | 5.37 | 1.92 | 1.22 | 3.41 | − | − | − | + |
| 1467108 | AA883160 | 4.29 | 2.67 | 1.42 | 1.61 | 3.02 | − | − | − | + |
| 1632161 | AI004930 | 39.75 | 38.39 | 11.08 | 1.04 | 3.59 | − | − | − | + |
| 950473 | AA599120 | 9.36 | 3.14 | 5.71 | 2.98 | 1.64 | − | + | − | − |
| 796885 | AA463200 | 9.64 | 6.52 | 3.08 | 1.48 | 3.13 | − | − | − | + |
| 854122 | AA669183 | 4.20 | 2.32 | 1.50 | 1.81 | 2.80 | − | − | + | + |
| 811911 | AA456271 | 11.89 | 6.59 | 4.25 | 1.80 | 2.80 | − | − | − | + |
| 383188 | AA074224 | 2.79 | 1.08 | 1.39 | 2.58 | 2.02 | − | + | − | − |
| 461336 | AA699770 | 3.42 | 4.00 | 0.91 | 0.85 | 3.74 | − | − | + | + |
| 324342 | W47576 | 4.41 | 4.29 | 1.24 | 1.03 | 3.57 | − | − | − | + |
| 470114 | AA029295 | 3.09 | 1.27 | 1.44 | 2.44 | 2.15 | − | + | − | + |
| 843352 | AA489343 | 28.44 | 25.71 | 8.17 | 1.11 | 3.48 | − | − | − | + |
| 129725 | R16957 | 17.94 | 10.53 | 6.22 | 1.70 | 2.88 | − | − | − | + |
| 121196 | T97170 | 22.80 | 22.77 | 6.36 | 1.00 | 3.58 | − | − | + | + |
| 838668 | AA457235 | 3.58 | 4.32 | 0.95 | 0.83 | 3.76 | − | − | + | + |
| 283142 | N51335 | 3.58 | 1.94 | 1.31 | 1.84 | 2.74 | − | − | − | + |
| 1636707 | AI017703 | 19.22 | 12.06 | 6.43 | 1.59 | 2.99 | − | − | − | + |
| 123980 | R01637 | 2.38 | 0.92 | 1.19 | 2.59 | 1.99 | − | + | − | − |
| 253009 | H88588 | 115.86 | 35.75 | 86.54 | 3.24 | 1.34 | + | + | − | − |
| 461489 | AA705058 | 0.92 | 0.61 | 0.30 | 1.51 | 3.07 | − | − | − | + |
| 856115 | AA630584 | 1.30 | 0.82 | 0.43 | 1.57 | 3.00 | − | − | + | − |
| 51151 | H18726 | 3.26 | 3.41 | 0.90 | 0.96 | 3.61 | − | − | + | + |
| 119403 | AI732248 | 1.22 | 1.22 | 0.34 | 1.00 | 3.57 | − | − | − | + |
| 813280 | AA455931 | 4.14 | 2.24 | 1.53 | 1.85 | 2.71 | − | − | − | + |
| 211206 | H67988 | 4.71 | 2.93 | 1.60 | 1.61 | 2.95 | − | − | − | + |
| 727251 | AA412053 | 34.32 | 29.87 | 10.07 | 1.15 | 3.41 | − | − | − | + |
| 1472150 | AA873577 | 35.19 | 14.23 | 16.93 | 2.47 | 2.08 | − | + | − | + |
| 627688 | AA197344 | 7.37 | 3.62 | 2.93 | 2.03 | 2.52 | + | − | + | − |
| 289288 | N68998 | 5.44 | 3.58 | 1.79 | 1.52 | 3.03 | − | − | − | + |
| 810725 | AA457717 | 3.27 | 1.79 | 1.20 | 1.83 | 2.72 | − | − | + | − |
| 243068 | H94409 | 9.34 | 5.76 | 3.20 | 1.62 | 2.92 | − | − | + | − |
| 769926 | AA430615 | 9.93 | 5.82 | 3.51 | 1.71 | 2.83 | − | − | + | − |
| 289818 | N62179 | 7.47 | 5.73 | 2.31 | 1.30 | 3.23 | − | − | − | + |
| 196345 | R92455 | 122.93 | 41.22 | 79.12 | 2.98 | 1.55 | − | + | − | − |
| 814080 | AA465424 | 8.19 | 4.01 | 3.29 | 2.04 | 2.49 | − | − | + | − |
| 74511 | T59099 | 5.23 | 3.37 | 1.75 | 1.55 | 2.98 | − | − | − | + |
| 730313 | AA412643 | 3.95 | 2.39 | 1.37 | 1.65 | 2.88 | − | − | − | + |
| 34869 | R44447 | 2.74 | 2.06 | 0.85 | 1.33 | 3.20 | − | − | + | − |
| 950574 | AA608514 | 28.90 | 11.69 | 14.05 | 2.47 | 2.06 | − | + | − | − |
| 77805 | T53907 | 15.55 | 9.75 | 5.31 | 1.59 | 2.93 | − | − | − | + |
| 1492304 | AA894577 | 4.23 | 2.66 | 1.44 | 1.59 | 2.93 | − | − | − | + |
| 263070 | N20052 | 1.17 | 0.90 | 0.37 | 1.31 | 3.21 | − | − | − | + |
| 951216 | AA620565 | 2.02 | 1.34 | 0.67 | 1.50 | 3.02 | − | − | − | + |
| 308726 | N93236 | 14.73 | 5.64 | 7.73 | 2.61 | 1.91 | − | + | − | − |
| 40136 | R53348 | 5.23 | 1.99 | 2.77 | 2.62 | 1.89 | + | − | − | − |
| 1461528 | AA883711 | 3.95 | 2.84 | 1.27 | 1.39 | 3.12 | − | − | + | + |
| 760148 | AA424344 | 2.93 | 1.10 | 1.58 | 2.66 | 1.85 | − | + | − | − |
| 430235 | AA010223 | 3.43 | 1.72 | 1.36 | 1.99 | 2.52 | − | − | − | + |
| 884867 | AA669443 | 24.71 | 16.78 | 8.14 | 1.47 | 3.03 | − | − | − | + |
| 744944 | AA625890 | 1.73 | 0.90 | 0.67 | 1.92 | 2.59 | − | − | − | + |
| 202904 | H54020 | 12.77 | 10.17 | 3.93 | 1.26 | 3.25 | − | − | − | + |
| 257382 | N30699 | 12.80 | 10.78 | 3.86 | 1.19 | 3.32 | − | − | + | + |
| 590390 | AA157150 | 6.42 | 3.41 | 2.45 | 1.88 | 2.62 | − | − | − | + |
| 460798 | AA708152 | 1.30 | 0.65 | 0.52 | 2.00 | 2.50 | − | − | − | + |
| 815164 | AA481135 | 0.88 | 0.71 | 0.27 | 1.25 | 3.25 | − | − | − | + |
| 214884 | H74119 | 3.55 | 2.09 | 1.27 | 1.70 | 2.79 | − | − | − | + |
| 782439 | AA431836 | 35.89 | 20.99 | 12.92 | 1.71 | 2.78 | − | − | − | + |
| 277596 | N57002 | 2.91 | 1.45 | 1.17 | 2.00 | 2.49 | − | − | + | − |
| 344432 | W73474 | 15.48 | 11.50 | 4.93 | 1.35 | 3.14 | − | − | − | + |
| 796341 | AA459750 | 7.16 | 4.60 | 2.44 | 1.56 | 2.93 | − | − | − | + |
| 731308 | AA416759 | 4.84 | 1.70 | 2.96 | 2.85 | 1.64 | − | + | − | − |
| 788518 | AA452566 | 13.97 | 9.65 | 4.61 | 1.45 | 3.03 | − | − | − | + |
| 201628 | R98008 | 7.51 | 5.63 | 2.39 | 1.33 | 3.14 | − | − | + | + |
| 1336262 | AA872057 | 35.80 | 21.64 | 12.69 | 1.65 | 2.82 | − | − | − | + |
| 898206 | AA598595 | 1.79 | 2.04 | 0.50 | 0.88 | 3.59 | − | − | − | + |
| 897547 | AA497010 | 15.69 | 7.46 | 6.62 | 2.10 | 2.37 | − | + | − | + |
| 489823 | AA099855 | 33.49 | 26.22 | 10.49 | 1.28 | 3.19 | − | − | + | + |
| 813825 | AA453713 | 0.52 | 0.34 | 0.18 | 1.52 | 2.95 | − | − | − | + |
| 781401 | AA430205 | 2.67 | 1.22 | 1.18 | 2.19 | 2.27 | − | − | + | − |
| 1492967 | AA886333 | 3.68 | 1.85 | 1.49 | 1.99 | 2.47 | + | − | + | − |
| 31225 | R41943 | 4.76 | 1.64 | 3.09 | 2.90 | 1.54 | − | + | − | − |
| 35807 | R46003 | 2.91 | 1.90 | 1.00 | 1.53 | 2.91 | − | − | − | + |
| 950450 | AA599094 | 1.77 | 1.02 | 0.66 | 1.75 | 2.70 | − | − | − | + |
| 814945 | AA465116 | 4.97 | 2.92 | 1.82 | 1.70 | 2.74 | − | − | − | + |
| 788205 | AA453926 | 13.21 | 6.75 | 5.32 | 1.96 | 2.48 | − | − | − | + |
| 789091 | AA452933 | 9.50 | 6.97 | 3.09 | 1.36 | 3.08 | − | − | + | + |
| 843140 | AA486516 | 14.94 | 10.35 | 4.99 | 1.44 | 3.00 | − | − | − | + |
| 838611 | AA456975 | 52.82 | 30.51 | 19.51 | 1.73 | 2.71 | − | − | − | + |
| 878403 | AA670353 | 7.20 | 6.82 | 2.13 | 1.06 | 3.38 | − | − | + | + |
| 868838 | AA775223 | 3.29 | 2.14 | 1.14 | 1.54 | 2.90 | − | − | + | − |
| 222527 | H84287 | 2.88 | 2.10 | 0.94 | 1.37 | 3.05 | − | − | − | + |
| 842767 | AA486300 | 2.15 | 1.48 | 0.73 | 1.46 | 2.96 | − | − | − | + |
| 897670 | AA496800 | 6.35 | 2.19 | 4.18 | 2.90 | 1.52 | − | + | − | − |
| 742581 | AA400247 | 1.20 | 1.08 | 0.36 | 1.11 | 3.31 | − | − | − | + |
| 52338 | H23277 | 2.91 | 1.24 | 1.41 | 2.35 | 2.07 | − | + | − | + |
| 590398 | AA148034 | 12.27 | 6.94 | 4.63 | 1.77 | 2.65 | − | − | − | + |
| 840620 | AA488036 | 13.81 | 8.57 | 4.92 | 1.61 | 2.81 | − | − | − | + |
| 854899 | AA630374 | 18.74 | 13.26 | 6.24 | 1.41 | 3.00 | − | − | − | + |
| 38253 | R35943 | 2.80 | 1.95 | 0.94 | 1.44 | 2.98 | − | − | − | + |
| 502369 | AA156940 | 1.41 | 0.77 | 0.55 | 1.82 | 2.59 | − | − | − | + |
| 343609 | W69460 | 1.58 | 0.58 | 0.94 | 2.73 | 1.68 | − | + | − | − |
| 701496 | AA286914 | 2.28 | 1.54 | 0.78 | 1.48 | 2.93 | − | − | + | + |
| 1493252 | AA886870 | 9.89 | 11.50 | 2.79 | 0.86 | 3.54 | − | − | + | + |
| 203350 | H54364 | 1.91 | 0.74 | 1.05 | 2.57 | 1.83 | − | + | − | − |
| 451707 | AA707659 | 7.59 | 5.80 | 2.45 | 1.31 | 3.09 | − | − | + | + |
| 22747 | T75072 | 37.20 | 31.66 | 11.55 | 1.18 | 3.22 | − | − | − | + |
| 815072 | AA465180 | 3.49 | 1.97 | 1.33 | 1.77 | 2.62 | − | − | + | − |
| 251731 | H96904 | 0.82 | 0.84 | 0.24 | 0.98 | 3.41 | − | − | − | + |
| 815563 | AA456833 | 4.86 | 3.88 | 1.55 | 1.25 | 3.13 | − | − | − | + |
| 627273 | AA190747 | 1.58 | 1.06 | 0.55 | 1.49 | 2.88 | − | − | − | + |
| 39821 | R53428 | 5.84 | 5.87 | 1.73 | 0.99 | 3.38 | − | − | − | + |
| 898210 | AA598597 | 8.13 | 8.02 | 2.42 | 1.01 | 3.35 | − | − | − | + |
| 46367 | H09959 | 1.63 | 0.62 | 0.94 | 2.62 | 1.74 | + | − | − | − |
| 28436 | R40663 | 0.89 | 0.37 | 0.46 | 2.43 | 1.93 | + | − | − | − |
| 259953 | N32594 | 1.46 | 1.97 | 0.40 | 0.74 | 3.63 | − | − | − | + |
| 307882 | W21373 | 7.09 | 4.18 | 2.66 | 1.69 | 2.67 | − | − | − | + |
| 713286 | AA283029 | 0.89 | 0.56 | 0.32 | 1.58 | 2.78 | − | − | − | + |
| 195903 | R92227 | 6.95 | 5.56 | 2.24 | 1.25 | 3.11 | − | − | − | + |
| 42076 | R60847 | 5.72 | 3.44 | 2.12 | 1.66 | 2.69 | − | − | − | + |
| 1501998 | AA887201 | 2.03 | 1.50 | 0.68 | 1.36 | 3.00 | − | − | − | + |
| 503468 | AA128297 | 51.40 | 43.89 | 16.15 | 1.17 | 3.18 | − | − | − | + |
| 838478 | AA457517 | 2.51 | 1.48 | 0.94 | 1.69 | 2.66 | − | − | − | + |
| 281191 | N50983 | 7.36 | 3.32 | 3.45 | 2.22 | 2.13 | − | + | − | + |
| 391984 | AI003625 | 4.56 | 2.74 | 1.70 | 1.66 | 2.68 | − | − | − | + |
| 1635665 | AI016688 | 5.13 | 4.14 | 1.65 | 1.24 | 3.11 | − | − | + | + |
| 49485 | H16600 | 0.54 | 0.37 | 0.19 | 1.46 | 2.89 | − | − | − | + |
| 429906 | AA033973 | 5.35 | 3.27 | 1.98 | 1.63 | 2.71 | − | − | − | + |
| 487773 | AA043347 | 29.51 | 20.98 | 10.06 | 1.41 | 2.93 | − | − | − | + |
| 701409 | AA287948 | 1.54 | 1.24 | 0.50 | 1.23 | 3.10 | − | − | − | + |
| 144977 | R78725 | 71.81 | 48.67 | 25.09 | 1.48 | 2.86 | − | − | − | + |
| 240961 | H91011 | 2.11 | 0.82 | 1.20 | 2.57 | 1.76 | − | + | − | − |
| 1049033 | AA778675 | 3.07 | 1.85 | 1.15 | 1.66 | 2.67 | − | − | − | + |
| 45515 | H08417 | 3.88 | 2.73 | 1.33 | 1.42 | 2.91 | − | − | + | + |
| 784116 | AA446743 | 0.26 | 0.10 | 0.15 | 2.61 | 1.72 | − | + | − | − |
| 796774 | AA443152 | 0.44 | 0.47 | 0.13 | 0.93 | 3.40 | − | − | − | + |
| 415978 | W86378 | 24.58 | 18.77 | 8.16 | 1.31 | 3.01 | − | − | − | + |
| 68636 | T49801 | 4.83 | 2.97 | 1.80 | 1.63 | 2.69 | − | − | − | + |
| 35812 | R14631 | 2.29 | 2.07 | 0.71 | 1.11 | 3.21 | − | − | − | + |
| 564846 | AA129297 | 1.90 | 1.12 | 0.72 | 1.69 | 2.63 | − | − | − | + |
| 503097 | AA151486 | 7.27 | 4.27 | 2.78 | 1.70 | 2.61 | − | − | + | − |
| 139113 | R63022 | 4.32 | 3.44 | 1.41 | 1.26 | 3.06 | − | − | + | + |
| 131621 | R23790 | 10.21 | 3.63 | 6.81 | 2.82 | 1.50 | − | + | − | − |
| 156386 | R73545 | 2.14 | 1.88 | 0.67 | 1.14 | 3.18 | − | − | − | + |
| 784264 | AA447021 | 5.67 | 3.30 | 2.18 | 1.72 | 2.60 | − | − | − | + |
| 291706 | N73480 | 2.41 | 1.00 | 1.27 | 2.41 | 1.90 | + | − | + | − |
| 310493 | W31074 | 7.56 | 7.25 | 2.32 | 1.04 | 3.26 | − | − | − | + |
| 784016 | AA443698 | 33.80 | 23.65 | 11.75 | 1.43 | 2.88 | − | − | − | + |
| 282144 | N51883 | 30.16 | 18.11 | 11.43 | 1.67 | 2.64 | − | − | + | − |
| 435341 | AA700736 | 1.73 | 1.11 | 0.63 | 1.56 | 2.74 | − | − | − | + |
| 252904 | H88485 | 5.40 | 2.26 | 2.83 | 2.39 | 1.91 | − | + | − | − |
| 433573 | AA701655 | 10.67 | 9.66 | 3.34 | 1.11 | 3.19 | − | − | − | + |
| 743041 | AA406040 | 6.48 | 3.98 | 2.43 | 1.63 | 2.67 | − | − | − | + |
| 897971 | AA598868 | 11.55 | 7.34 | 4.24 | 1.57 | 2.73 | − | − | − | + |
| 827168 | AA521292 | 6.51 | 2.85 | 3.24 | 2.29 | 2.01 | + | − | + | − |
| 141361 | R64454 | 1.28 | 0.68 | 0.54 | 1.90 | 2.40 | − | − | + | − |
| 767298 | AA418383 | 19.37 | 16.76 | 6.18 | 1.16 | 3.14 | − | − | − | + |
| 436106 | AA701981 | 4.14 | 2.42 | 1.60 | 1.71 | 2.58 | − | − | − | + |
| 884388 | AA629554 | 4.89 | 3.97 | 1.60 | 1.23 | 3.06 | − | − | − | + |
| 46294 | H09132 | 1.03 | 0.35 | 0.78 | 2.97 | 1.31 | + | + | − | − |
| 253253 | H89104 | 1.73 | 1.30 | 0.58 | 1.33 | 2.95 | − | − | − | + |
| 1031346 | AA609106 | 3.21 | 2.11 | 1.16 | 1.52 | 2.76 | − | − | − | + |
| 429574 | AA011445 | 3.77 | 1.23 | 3.11 | 3.06 | 1.21 | − | + | − | − |
| 52327 | H23459 | 5.70 | 4.77 | 1.85 | 1.19 | 3.08 | − | − | − | + |
| 810741 | AA457725 | 72.43 | 48.42 | 26.10 | 1.50 | 2.78 | − | − | − | + |
| 155838 | R72215 | 2.53 | 1.96 | 0.85 | 1.29 | 2.97 | − | − | + | − |
| 135083 | R33917 | 17.07 | 17.59 | 5.18 | 0.97 | 3.29 | − | − | − | + |
| 271472 | N35020 | 11.80 | 7.84 | 4.28 | 1.50 | 2.76 | − | − | − | + |
| 29964 | R14699 | 1.12 | 0.63 | 0.45 | 1.79 | 2.47 | − | − | − | + |
| 269300 | N35863 | 10.64 | 7.57 | 3.73 | 1.41 | 2.85 | − | − | − | + |
| 429678 | AA011593 | 3.12 | 0.86 | 4.85 | 3.61 | 0.64 | + | + | − | − |
| 898096 | AA598795 | 4.06 | 2.82 | 1.45 | 1.44 | 2.81 | − | − | − | + |
| 730554 | AA436031 | 11.89 | 8.38 | 4.20 | 1.42 | 2.83 | − | − | − | + |
| 260628 | H97565 | 8.94 | 7.49 | 2.93 | 1.19 | 3.05 | − | − | − | + |
| 730412 | AA469966 | 1.25 | 1.00 | 0.42 | 1.25 | 3.00 | − | − | − | + |
| 825771 | AA491312 | 12.13 | 9.01 | 4.19 | 1.35 | 2.90 | − | − | + | − |
| 342211 | W63789 | 1.92 | 1.55 | 0.64 | 1.24 | 3.00 | − | − | − | + |
| 845609 | AA644345 | 15.29 | 12.54 | 5.07 | 1.22 | 3.02 | − | − | − | + |
| 814154 | AA496252 | 2.29 | 1.38 | 0.89 | 1.67 | 2.57 | − | − | + | − |
| 897730 | AA598987 | 41.12 | 27.32 | 15.07 | 1.50 | 2.73 | − | − | − | + |
| 141336 | R63823 | 2.12 | 1.03 | 0.97 | 2.05 | 2.18 | + | − | + | − |
| 263894 | N28522 | 0.46 | 0.42 | 0.15 | 1.10 | 3.13 | − | − | − | + |
| 252864 | H88424 | 1.20 | 1.00 | 0.40 | 1.20 | 3.03 | − | − | − | + |
| 291057 | N72115 | 1.34 | 0.61 | 0.66 | 2.18 | 2.04 | − | + | − | − |
| 752690 | AA417805 | 6.05 | 3.90 | 2.27 | 1.55 | 2.67 | − | − | − | + |
| 132911 | R26186 | 52.56 | 22.96 | 27.26 | 2.29 | 1.93 | − | + | − | + |
| 489663 | AA099404 | 2.11 | 1.57 | 0.73 | 1.34 | 2.87 | − | − | − | + |
| 430614 | AA678065 | 2.38 | 1.00 | 1.30 | 2.39 | 1.83 | − | + | − | − |
| 1415981 | AA947922 | 4.13 | 3.78 | 1.32 | 1.09 | 3.12 | − | − | − | + |
| 810027 | AA464995 | 0.44 | 0.43 | 0.14 | 1.04 | 3.16 | − | − | − | + |
| 768469 | AA495962 | 4.17 | 3.13 | 1.45 | 1.33 | 2.87 | − | − | − | + |
| 271076 | N42970 | 4.31 | 3.13 | 1.53 | 1.38 | 2.82 | − | − | − | + |
| 759948 | AA424045 | 4.05 | 3.18 | 1.39 | 1.27 | 2.91 | − | − | + | − |
| 529861 | AA070997 | 5.75 | 3.77 | 2.16 | 1.52 | 2.66 | − | − | − | + |
| 838628 | AA457082 | 2.82 | 2.95 | 0.87 | 0.96 | 3.23 | − | − | − | + |
| 590853 | AA161283 | 0.66 | 0.64 | 0.21 | 1.04 | 3.14 | − | − | − | + |
| 36387 | R46816 | 3.19 | 3.07 | 1.02 | 1.04 | 3.14 | − | − | − | + |
| 950464 | AA599099 | 9.75 | 5.67 | 3.98 | 1.72 | 2.45 | + | − | + | − |
| 898227 | AA598625 | 2.02 | 1.65 | 0.68 | 1.22 | 2.95 | − | − | − | + |
| 811757 | AA463449 | 1.59 | 1.01 | 0.61 | 1.57 | 2.59 | − | − | − | + |
| 272049 | N35369 | 6.87 | 3.74 | 2.95 | 1.84 | 2.33 | − | − | − | + |
| 328613 | W45285 | 0.46 | 0.40 | 0.15 | 1.13 | 3.03 | − | − | − | + |
| 461488 | AA705047 | 0.55 | 0.43 | 0.19 | 1.27 | 2.89 | − | − | − | + |
| 1593502 | AA989139 | 2.25 | 1.64 | 0.81 | 1.37 | 2.79 | − | − | − | + |
| 701402 | AA287936 | 4.23 | 3.51 | 1.43 | 1.20 | 2.96 | − | − | + | + |
| 234331 | H93191 | 3.16 | 2.04 | 1.21 | 1.55 | 2.61 | − | − | − | + |
| 251645 | H97033 | 0.50 | 0.47 | 0.16 | 1.06 | 3.09 | − | − | − | + |
| 50892 | H18560 | 7.95 | 7.40 | 2.58 | 1.08 | 3.08 | − | − | − | + |
| 826335 | AA521108 | 2.24 | 1.62 | 0.81 | 1.39 | 2.77 | − | − | − | + |
| 302955 | N91117 | 10.63 | 10.42 | 3.39 | 1.02 | 3.14 | − | − | + | + |
| 1475738 | AA872704 | 238.01 | 171.23 | 86.12 | 1.39 | 2.76 | − | − | − | + |
| 884894 | AA669452 | 41.65 | 28.65 | 15.44 | 1.45 | 2.70 | − | − | − | + |
| 1638479 | AI016074 | 3.27 | 2.02 | 1.30 | 1.62 | 2.53 | − | − | − | + |
| 202154 | H52503 | 29.46 | 16.52 | 12.46 | 1.78 | 2.36 | − | − | + | − |
| 197821 | R93744 | 5.95 | 4.32 | 2.15 | 1.38 | 2.77 | − | − | + | + |
| 951022 | AA620421 | 3.02 | 1.90 | 1.18 | 1.59 | 2.55 | − | − | − | + |
| 356535 | AA633577 | 1.40 | 0.50 | 1.04 | 2.79 | 1.35 | − | + | − | − |
| 841140 | AA487020 | 2.47 | 3.20 | 0.73 | 0.77 | 3.36 | − | − | − | + |
| 197765 | R93767 | 8.83 | 3.78 | 4.92 | 2.34 | 1.79 | + | − | − | − |
| 125308 | R05886 | 2.61 | 0.93 | 1.97 | 2.80 | 1.33 | − | + | − | − |
| 323611 | W44508 | 1.25 | 0.50 | 0.76 | 2.48 | 1.64 | − | + | − | − |
| 853998 | AA668897 | 12.39 | 4.81 | 8.02 | 2.58 | 1.55 | − | + | − | − |
| 884766 | AA629591 | 9.95 | 9.24 | 3.27 | 1.08 | 3.04 | − | − | + | + |
| 813712 | AA453849 | 7.62 | 7.57 | 2.45 | 1.01 | 3.11 | − | − | + | + |
| 811097 | AA485675 | 2.59 | 1.28 | 1.23 | 2.02 | 2.10 | + | − | + | − |
| 243321 | H95088 | 2.79 | 1.90 | 1.05 | 1.47 | 2.65 | − | − | + | − |
| 856575 | AA633658 | 2.75 | 2.77 | 0.88 | 0.99 | 3.12 | − | − | + | + |
| 360232 | AA013099 | 2.20 | 1.94 | 0.74 | 1.13 | 2.98 | − | − | − | + |
| 26185 | R20616 | 1.15 | 0.80 | 0.43 | 1.43 | 2.69 | − | − | − | + |
| 469685 | AA027912 | 4.95 | 4.33 | 1.67 | 1.14 | 2.97 | − | − | − | + |
| 841501 | AA487382 | 5.94 | 4.80 | 2.07 | 1.24 | 2.88 | − | − | − | + |
| 428215 | AA001745 | 42.11 | 30.60 | 15.40 | 1.38 | 2.73 | − | − | − | + |
| 825356 | AA504492 | 1.18 | 0.90 | 0.42 | 1.32 | 2.79 | − | − | − | + |
| 884480 | AA629719 | 11.68 | 10.42 | 3.91 | 1.12 | 2.99 | − | − | − | + |
| 813179 | AA456717 | 6.60 | 2.40 | 4.88 | 2.75 | 1.35 | + | − | − | − |
| 645368 | AA206267 | 8.48 | 7.77 | 2.82 | 1.09 | 3.01 | − | − | − | + |
| 666371 | AA232645 | 0.85 | 1.07 | 0.26 | 0.79 | 3.30 | − | − | − | + |
| 768179 | AA426629 | 3.80 | 3.11 | 1.32 | 1.22 | 2.87 | − | − | − | + |
| 1420830 | AA826324 | 3.06 | 2.56 | 1.06 | 1.19 | 2.90 | − | − | − | + |
| 246541 | N77514 | 10.77 | 8.71 | 3.78 | 1.24 | 2.85 | − | − | − | + |
| 742695 | AA401521 | 1.83 | 1.55 | 0.63 | 1.18 | 2.90 | − | − | − | + |
| 451769 | AA706795 | 5.25 | 5.71 | 1.66 | 0.92 | 3.16 | − | − | + | + |
| 827132 | AA521232 | 6.63 | 4.13 | 2.68 | 1.60 | 2.48 | − | − | − | + |
| 460218 | AA677457 | 2.10 | 1.12 | 0.95 | 1.87 | 2.21 | − | − | − | + |
| 40075 | R52015 | 3.13 | 3.07 | 1.03 | 1.02 | 3.05 | − | − | − | + |
| 127928 | R08932 | 3.67 | 2.74 | 1.35 | 1.34 | 2.73 | − | − | − | + |
| 279785 | N48356 | 0.91 | 0.66 | 0.34 | 1.38 | 2.68 | − | − | + | − |
| 1591622 | AA983467 | 49.35 | 42.75 | 16.96 | 1.15 | 2.91 | − | − | − | + |
| 824640 | AA482150 | 6.96 | 6.37 | 2.35 | 1.09 | 2.97 | − | − | − | + |
| 1635350 | AI014387 | 10.17 | 8.91 | 3.48 | 1.14 | 2.92 | − | − | + | − |
| 855723 | AA663960 | 6.73 | 7.60 | 2.12 | 0.89 | 3.17 | − | − | − | + |
| 1475746 | AA873762 | 12.48 | 10.32 | 4.39 | 1.21 | 2.84 | − | − | − | + |
| 845502 | AI791165 | 3.87 | 3.96 | 1.26 | 0.98 | 3.07 | − | − | − | + |
| 897983 | AA598874 | 26.99 | 22.87 | 9.42 | 1.18 | 2.86 | − | − | − | + |
| 824066 | AA490734 | 4.08 | 3.05 | 1.51 | 1.34 | 2.70 | − | − | + | − |
| 627687 | AA196287 | 56.19 | 60.68 | 18.11 | 0.93 | 3.10 | − | − | − | + |
| 32887 | R43699 | 3.04 | 2.20 | 1.15 | 1.39 | 2.64 | − | − | − | + |
| 433350 | AA700604 | 28.82 | 38.07 | 8.83 | 0.76 | 3.26 | − | − | + | − |
| 812989 | AA464623 | 2.18 | 1.75 | 0.79 | 1.25 | 2.77 | − | − | − | + |
| 244154 | N52450 | 4.94 | 3.91 | 1.79 | 1.26 | 2.76 | − | − | − | + |
| 278687 | N62924 | 13.10 | 9.87 | 4.88 | 1.33 | 2.69 | − | − | − | + |
| 322537 | W15263 | 3.18 | 2.15 | 1.26 | 1.48 | 2.53 | − | − | + | − |
| 399532 | AA733038 | 6.74 | 4.35 | 2.74 | 1.55 | 2.46 | − | − | − | + |
| 854570 | AA669124 | 4.11 | 2.43 | 1.78 | 1.69 | 2.31 | − | − | − | + |
| 1411726 | AA856556 | 30.68 | 28.07 | 10.55 | 1.09 | 2.91 | − | − | − | + |
| 1475881 | AA872153 | 14.80 | 6.17 | 9.24 | 2.40 | 1.60 | − | + | − | − |
| 80764 | T63027 | 26.16 | 26.33 | 8.71 | 0.99 | 3.00 | − | − | − | + |
| 825012 | AA489194 | 4.46 | 3.05 | 1.76 | 1.46 | 2.53 | − | − | − | + |
| 51981 | H23422 | 19.62 | 13.04 | 7.89 | 1.50 | 2.49 | − | − | − | + |
| 1591264 | AA955007 | 3.18 | 2.86 | 1.11 | 1.11 | 2.88 | − | − | − | + |
| 898265 | AA598965 | 20.16 | 18.09 | 7.03 | 1.11 | 2.87 | − | − | − | + |
| 511459 | AA115310 | 37.04 | 46.42 | 11.64 | 0.80 | 3.18 | − | − | − | + |
| 296429 | W00890 | 5.99 | 4.85 | 2.18 | 1.23 | 2.75 | − | − | − | + |
| 754449 | AA410480 | 1.78 | 1.58 | 0.63 | 1.13 | 2.85 | − | − | − | + |
| 813149 | AA456298 | 6.11 | 6.08 | 2.05 | 1.00 | 2.97 | − | − | + | + |
| 741988 | AA402915 | 1.74 | 1.20 | 0.69 | 1.45 | 2.52 | − | − | − | + |
| 825842 | AA504772 | 6.00 | 6.39 | 1.98 | 0.94 | 3.03 | − | − | + | + |
| 1492412 | AA878561 | 236.26 | 205.84 | 83.73 | 1.15 | 2.82 | − | − | − | + |
| 1422794 | AA827405 | 2.62 | 1.78 | 1.05 | 1.47 | 2.49 | − | − | − | + |
| 731104 | AA421455 | 0.65 | 0.64 | 0.22 | 1.02 | 2.93 | − | − | − | + |
| 80292 | T64433 | 2.18 | 1.64 | 0.83 | 1.33 | 2.62 | − | − | − | + |
| 969844 | AA663826 | 9.12 | 11.76 | 2.88 | 0.78 | 3.17 | − | − | + | + |
| 75059 | T50370 | 21.82 | 10.94 | 11.17 | 2.00 | 1.95 | − | + | − | + |
| 303139 | W19519 | 2.17 | 1.22 | 1.00 | 1.77 | 2.17 | − | − | + | − |
| 48033 | H11760 | 0.51 | 0.50 | 0.17 | 1.02 | 2.92 | − | − | − | + |
| 282720 | N50079 | 8.31 | 6.24 | 3.18 | 1.33 | 2.61 | − | − | − | + |
| 291974 | N73091 | 57.24 | 40.53 | 22.63 | 1.41 | 2.53 | − | − | − | + |
| 307029 | N89671 | 56.38 | 44.33 | 21.22 | 1.27 | 2.66 | − | − | − | + |
| 593520 | AA165402 | 6.25 | 6.33 | 2.13 | 0.99 | 2.93 | − | − | − | + |
| 1471829 | AA873351 | 99.54 | 79.29 | 37.33 | 1.26 | 2.67 | − | − | + | − |
| 461955 | AA779968 | 1.94 | 0.91 | 1.09 | 2.13 | 1.78 | − | + | − | − |
| 27072 | R37780 | 0.29 | 0.27 | 0.10 | 1.07 | 2.85 | − | − | − | + |
| 282895 | N51226 | 3.91 | 1.96 | 2.03 | 1.99 | 1.93 | + | − | + | − |
| 866866 | AA679341 | 3.00 | 2.04 | 1.23 | 1.47 | 2.45 | − | − | − | + |
| 878373 | AA670330 | 31.06 | 35.83 | 10.19 | 0.87 | 3.05 | − | − | − | + |
| 471641 | AA034945 | 4.79 | 2.74 | 2.22 | 1.75 | 2.16 | − | + | − | + |
| 447568 | AA702422 | 9.00 | 7.57 | 3.31 | 1.19 | 2.72 | − | − | − | + |
| 866276 | AA663160 | 1.94 | 1.83 | 0.68 | 1.06 | 2.84 | − | − | − | + |
| 246800 | N59078 | 6.67 | 7.15 | 2.25 | 0.93 | 2.96 | − | − | − | + |
| 565062 | AA133896 | 1.38 | 1.65 | 0.45 | 0.84 | 3.05 | − | − | − | + |
| 42313 | R60946 | 1.33 | 1.07 | 0.51 | 1.24 | 2.64 | − | − | − | + |
| 753917 | AA479100 | 6.49 | 5.74 | 2.36 | 1.13 | 2.75 | − | − | − | + |
| 234036 | H68988 | 4.77 | 3.83 | 1.81 | 1.24 | 2.64 | − | − | − | + |
| 884333 | AA629528 | 4.10 | 2.83 | 1.69 | 1.45 | 2.43 | − | − | − | + |
| 758347 | AA403072 | 2.75 | 2.38 | 1.01 | 1.16 | 2.72 | − | − | − | + |
| 795738 | AA460286 | 2.06 | 1.70 | 0.77 | 1.21 | 2.66 | − | − | − | + |
| 72616 | T51995 | 1.67 | 1.29 | 0.65 | 1.29 | 2.58 | − | − | − | + |
| 251753 | H96908 | 2.29 | 3.06 | 0.74 | 0.75 | 3.12 | − | − | + | − |
| 950607 | AA608548 | 19.79 | 11.09 | 9.50 | 1.78 | 2.08 | − | − | + | − |
| 825859 | AA504784 | 1.64 | 1.22 | 0.65 | 1.35 | 2.52 | − | − | − | + |
| 161456 | H25546 | 13.35 | 10.78 | 5.08 | 1.24 | 2.63 | − | − | + | − |
| 897577 | AA496881 | 0.52 | 0.50 | 0.18 | 1.03 | 2.82 | − | − | − | + |
| 1599489 | AA961361 | 5.51 | 4.50 | 2.10 | 1.22 | 2.62 | − | − | − | + |
| 195652 | R89403 | 18.03 | 15.86 | 6.65 | 1.14 | 2.71 | − | − | − | + |
| 431597 | AA676422 | 5.45 | 2.95 | 2.73 | 1.85 | 2.00 | + | − | + | − |
| 486340 | AA044123 | 16.47 | 11.82 | 6.73 | 1.39 | 2.45 | − | − | − | + |
| 742806 | AA400362 | 1.76 | 2.10 | 0.59 | 0.84 | 2.99 | − | − | − | + |
| 770935 | AA433877 | 86.21 | 50.78 | 40.44 | 1.70 | 2.13 | − | − | − | + |
| 53165 | R16153 | 0.89 | 0.87 | 0.32 | 1.02 | 2.80 | − | − | − | + |
| 307231 | N93428 | 2.04 | 1.57 | 0.81 | 1.30 | 2.53 | − | − | + | − |
| 826459 | AA521083 | 12.36 | 7.59 | 5.65 | 1.63 | 2.19 | − | − | − | + |
| 868308 | AA634008 | 65.99 | 60.02 | 24.35 | 1.10 | 2.71 | − | − | − | + |
| 1056203 | AA621031 | 1.92 | 0.89 | 1.16 | 2.15 | 1.65 | + | − | − | − |
| 486175 | AA043133 | 10.39 | 9.02 | 3.91 | 1.15 | 2.66 | − | − | + | − |
| 81484 | T63342 | 10.87 | 5.15 | 6.40 | 2.11 | 1.70 | − | + | − | − |
| 429047 | AA007509 | 3.17 | 1.48 | 1.90 | 2.14 | 1.67 | − | + | − | − |
| 783697 | AA446839 | 3.19 | 2.89 | 1.18 | 1.10 | 2.70 | − | − | − | + |
| 784128 | AA446759 | 1.83 | 1.82 | 0.65 | 1.00 | 2.80 | − | − | + | − |
| 253865 | N22007 | 1.68 | 1.49 | 0.63 | 1.13 | 2.67 | − | − | + | + |
| 1291658 | AA776810 | 2.05 | 1.00 | 1.17 | 2.05 | 1.75 | − | + | − | − |
| 595604 | AA167269 | 9.41 | 8.19 | 3.56 | 1.15 | 2.65 | − | − | − | + |
| 784129 | AA432085 | 6.53 | 5.53 | 2.50 | 1.18 | 2.61 | − | − | + | − |
| 251452 | H97993 | 3.12 | 2.79 | 1.17 | 1.12 | 2.67 | − | − | + | − |
| 767346 | AA418557 | 4.50 | 3.43 | 1.82 | 1.31 | 2.47 | − | − | + | − |
| 138865 | R62742 | 5.42 | 4.35 | 2.14 | 1.25 | 2.53 | − | − | − | + |
| 770983 | AA427401 | 7.24 | 6.59 | 2.70 | 1.10 | 2.68 | − | − | + | − |
| 43705 | H05706 | 0.35 | 0.33 | 0.13 | 1.07 | 2.71 | − | − | − | + |
| 76005 | T47291 | 2.35 | 1.80 | 0.95 | 1.31 | 2.46 | − | − | − | + |
| 156045 | R72518 | 2.01 | 1.50 | 0.83 | 1.34 | 2.43 | − | − | − | + |
| 784085 | AA446639 | 3.93 | 3.17 | 1.56 | 1.24 | 2.52 | − | − | − | + |
| 418053 | W90732 | 7.57 | 7.14 | 2.80 | 1.06 | 2.70 | − | − | − | + |
| 322192 | W37779 | 7.97 | 9.95 | 2.69 | 0.80 | 2.96 | − | − | + | + |
| 309864 | W23847 | 2.49 | 1.62 | 1.12 | 1.54 | 2.22 | + | − | + | − |
| 44975 | H08820 | 8.91 | 10.72 | 3.05 | 0.83 | 2.92 | − | − | − | + |
| 825719 | AA504834 | 0.76 | 0.67 | 0.29 | 1.14 | 2.62 | − | − | − | + |
| 291394 | W03029 | 6.46 | 4.22 | 2.92 | 1.53 | 2.22 | − | − | + | − |
| 796313 | AA461311 | 0.78 | 0.80 | 0.28 | 0.98 | 2.77 | − | − | − | + |
| 810734 | AA480820 | 2.46 | 1.75 | 1.05 | 1.40 | 2.35 | − | − | − | + |
| 488033 | AA045792 | 3.14 | 2.24 | 1.34 | 1.40 | 2.34 | − | − | − | + |
| 884546 | AA629808 | 156.92 | 121.14 | 64.25 | 1.30 | 2.44 | − | − | − | + |
| 41411 | R56885 | 112.58 | 99.23 | 43.25 | 1.13 | 2.60 | − | − | − | + |
| 247546 | N58107 | 11.16 | 8.98 | 4.48 | 1.24 | 2.49 | − | − | + | − |
| 743081 | AA496173 | 3.75 | 3.51 | 1.41 | 1.07 | 2.67 | − | − | − | + |
| 37665 | R61374 | 1.01 | 0.72 | 0.43 | 1.40 | 2.33 | − | − | − | + |
| 563451 | AA113347 | 4.17 | 1.82 | 2.90 | 2.29 | 1.44 | − | + | − | − |
| 725503 | AA292995 | 4.55 | 3.15 | 1.99 | 1.45 | 2.28 | − | − | − | + |
| 1522799 | AA902249 | 1.23 | 1.56 | 0.42 | 0.79 | 2.94 | − | − | − | + |
| 268178 | N35772 | 28.02 | 27.47 | 10.37 | 1.02 | 2.70 | − | − | − | + |
| 757222 | AA496148 | 5.14 | 3.76 | 2.18 | 1.37 | 2.36 | − | − | + | − |
| 1475028 | AA857413 | 249.73 | 233.03 | 94.34 | 1.07 | 2.65 | − | − | − | + |
| 840530 | AA487934 | 4.05 | 3.61 | 1.56 | 1.12 | 2.60 | − | − | − | + |
| 950989 | AA620404 | 2.48 | 1.95 | 1.02 | 1.28 | 2.44 | − | − | − | + |
| 756556 | AA481438 | 17.53 | 6.47 | 17.49 | 2.71 | 1.00 | + | − | − | − |
| 868396 | AA634164 | 6.77 | 5.15 | 2.83 | 1.32 | 2.39 | − | − | + | + |
| 857681 | AA633768 | 54.39 | 52.93 | 20.36 | 1.03 | 2.67 | − | − | − | + |
| 1555523 | AA974971 | 1.21 | 0.85 | 0.53 | 1.41 | 2.28 | − | − | + | − |
| 206785 | R98047 | 6.05 | 5.71 | 2.30 | 1.06 | 2.63 | − | − | − | + |
| 781061 | AA446479 | 0.73 | 0.44 | 0.35 | 1.64 | 2.05 | − | − | − | + |
| 345056 | W72798 | 0.87 | 0.35 | 0.71 | 2.47 | 1.22 | − | + | − | − |
| 897625 | AA496736 | 2.58 | 3.15 | 0.90 | 0.82 | 2.87 | − | − | − | + |
| 376764 | AA046321 | 2.58 | 2.44 | 0.98 | 1.06 | 2.63 | − | − | − | + |
| 452936 | AA778869 | 5.15 | 4.43 | 2.04 | 1.16 | 2.52 | − | − | − | + |
| 76605 | T51043 | 2.48 | 2.10 | 0.99 | 1.18 | 2.50 | − | − | − | + |
| 859832 | AA668531 | 2.57 | 2.48 | 0.97 | 1.04 | 2.64 | − | − | − | + |
| 809995 | AA454862 | 2.80 | 2.07 | 1.21 | 1.35 | 2.32 | − | − | − | + |
| 632137 | AA167728 | 0.80 | 0.57 | 0.35 | 1.41 | 2.27 | − | − | − | + |
| 122183 | T98628 | 7.61 | 10.97 | 2.56 | 0.69 | 2.98 | − | − | − | + |
| 284545 | N64762 | 1.10 | 0.77 | 0.49 | 1.42 | 2.24 | − | − | − | + |
| 815772 | AA485024 | 1.44 | 1.12 | 0.61 | 1.29 | 2.38 | − | − | − | + |
| 629498 | AA192765 | 3.73 | 2.69 | 1.64 | 1.39 | 2.28 | − | − | − | + |
| 243784 | N33927 | 2.99 | 2.12 | 1.33 | 1.41 | 2.25 | − | − | − | + |
| 838504 | AA457529 | 41.97 | 41.10 | 15.95 | 1.02 | 2.63 | − | − | − | + |
| 450711 | AA704459 | 1.29 | 1.51 | 0.46 | 0.86 | 2.79 | − | − | − | + |
| 83011 | T70522 | 32.97 | 35.26 | 12.18 | 0.93 | 2.71 | − | − | − | + |
| 866882 | AA679352 | 10.73 | 11.79 | 3.94 | 0.91 | 2.73 | − | − | − | + |
| 277761 | N49605 | 2.43 | 1.65 | 1.12 | 1.47 | 2.17 | − | − | − | + |
| 489722 | AA099593 | 4.03 | 4.49 | 1.47 | 0.90 | 2.74 | − | − | − | + |
| 796527 | AA463822 | 1.35 | 1.17 | 0.55 | 1.15 | 2.48 | − | − | + | − |
| 33045 | R19478 | 0.63 | 0.43 | 0.29 | 1.47 | 2.15 | − | − | + | − |
| 137984 | R63129 | 1.63 | 1.42 | 0.66 | 1.15 | 2.47 | − | − | + | − |
| 1574594 | AA968896 | 3.08 | 2.54 | 1.28 | 1.21 | 2.40 | − | − | − | + |
| 138861 | R62780 | 1.72 | 1.35 | 0.74 | 1.27 | 2.33 | − | − | − | + |
| 289645 | N62866 | 0.92 | 0.67 | 0.42 | 1.38 | 2.21 | − | − | + | − |
| 140792 | R67081 | 1.66 | 0.85 | 1.02 | 1.96 | 1.63 | + | − | − | − |
| 130136 | R20813 | 0.29 | 0.22 | 0.12 | 1.29 | 2.29 | − | − | + | − |
| 503741 | AA131466 | 3.64 | 4.28 | 1.33 | 0.85 | 2.74 | − | − | + | + |
| 827144 | AA521243 | 2.52 | 2.65 | 0.96 | 0.95 | 2.63 | − | − | − | + |
| 841348 | AA487650 | 11.96 | 14.60 | 4.33 | 0.82 | 2.76 | − | − | − | + |
| 949940 | AA599178 | 12.52 | 17.40 | 4.38 | 0.72 | 2.86 | − | − | − | + |
| 450464 | AA682819 | 3.97 | 2.31 | 2.13 | 1.72 | 1.86 | − | − | − | + |
| 743211 | AA400214 | 3.84 | 1.71 | 2.87 | 2.24 | 1.34 | + | − | − | − |
| 460164 | AA676920 | 0.80 | 0.68 | 0.33 | 1.17 | 2.41 | − | − | − | + |
| 856174 | AA630620 | 0.25 | 0.18 | 0.11 | 1.36 | 2.21 | − | − | − | + |
| 142326 | R70649 | 1.49 | 0.78 | 0.90 | 1.92 | 1.65 | + | − | − | − |
| 1475842 | AA872143 | 224.84 | 89.33 | 214.43 | 2.52 | 1.05 | + | − | − | − |
| 731255 | AA420992 | 2.04 | 1.38 | 0.98 | 1.48 | 2.09 | − | − | + | − |
| 897199 | AA677499 | 14.78 | 16.78 | 5.51 | 0.88 | 2.68 | − | − | − | + |
| 244202 | N52973 | 9.25 | 9.97 | 3.51 | 0.93 | 2.63 | − | − | − | + |
| 838568 | AA456931 | 53.19 | 53.66 | 20.71 | 0.99 | 2.57 | − | − | − | + |
| 844768 | AA774230 | 6.47 | 4.45 | 3.08 | 1.45 | 2.10 | − | − | − | + |
| 251417 | H96504 | 0.69 | 0.68 | 0.27 | 1.01 | 2.52 | − | − | − | + |
| 898251 | AA598675 | 2.02 | 3.02 | 0.71 | 0.67 | 2.86 | − | − | − | + |
| 452668 | AA779221 | 4.95 | 6.83 | 1.77 | 0.72 | 2.80 | − | − | + | − |
| 809722 | AA455482 | 50.59 | 51.18 | 19.96 | 0.99 | 2.53 | − | − | − | + |
| 278531 | N66158 | 57.48 | 74.35 | 20.99 | 0.77 | 2.74 | − | − | − | + |
| 744905 | AA625788 | 2.18 | 2.08 | 0.89 | 1.05 | 2.46 | − | − | − | + |
| 251727 | H97851 | 4.88 | 3.87 | 2.17 | 1.26 | 2.25 | − | − | − | + |
| 884892 | AA669451 | 8.02 | 8.08 | 3.19 | 0.99 | 2.51 | − | − | − | + |
| 487373 | AA046489 | 54.67 | 58.82 | 21.22 | 0.93 | 2.58 | − | − | − | + |
| 842863 | AA486403 | 11.11 | 10.81 | 4.49 | 1.03 | 2.48 | − | − | − | + |
| 767988 | AA418943 | 3.64 | 3.03 | 1.58 | 1.20 | 2.30 | − | − | − | + |
| 897448 | AA489478 | 31.17 | 29.51 | 12.76 | 1.06 | 2.44 | − | − | − | + |
| 1558642 | AA976544 | 3.11 | 3.33 | 1.21 | 0.93 | 2.56 | − | − | − | + |
| 46620 | H10036 | 5.25 | 2.02 | 6.01 | 2.61 | 0.87 | − | + | − | − |
| 855336 | AA630376 | 5.69 | 5.26 | 2.37 | 1.08 | 2.40 | − | − | − | + |
| 1573087 | AA970720 | 2.41 | 3.27 | 0.88 | 0.74 | 2.73 | − | − | − | + |
| 811880 | AA454634 | 3.13 | 4.22 | 1.15 | 0.74 | 2.72 | − | − | − | + |
| 841615 | AA487680 | 12.16 | 5.15 | 11.09 | 2.36 | 1.10 | + | − | − | − |
| 149895 | H00752 | 6.05 | 4.56 | 2.84 | 1.33 | 2.13 | − | − | + | − |
| 461699 | AA682274 | 3.44 | 1.35 | 3.84 | 2.55 | 0.90 | + | − | − | − |
| 743574 | AA609456 | 6.42 | 4.46 | 3.21 | 1.44 | 2.00 | − | − | − | + |
| 323850 | AA490456 | 0.78 | 0.30 | 0.32 | 0.98 | 2.46 | − | − | + | − |
| 435330 | AA699926 | 10.36 | 11.94 | 4.03 | 0.87 | 2.57 | − | − | + | − |
| 84078 | H0999 | 0.34 | 0.26 | 0.16 | 1.29 | 2.14 | − | − | − | + |
| 415415 | W81118 | 9.68 | 10.64 | 3.83 | 0.91 | 2.52 | − | − | − | + |
| 285226 | N66278 | 25.74 | 40.08 | 9.23 | 0.64 | 2.79 | − | − | + | − |
| 41207 | R56771 | 2.05 | 2.42 | 0.80 | 0.85 | 2.57 | − | − | − | + |
| 122239 | T98662 | 6.50 | 5.28 | 2.98 | 1.23 | 2.18 | − | − | − | + |
| 284865 | N66734 | 2.87 | 2.71 | 1.22 | 1.06 | 2.34 | − | − | − | + |
| 867717 | AA780841 | 5.61 | 3.47 | 3.15 | 1.61 | 1.78 | − | − | − | + |
| 50562 | H16903 | 34.20 | 30.09 | 15.14 | 1.14 | 2.26 | − | − | + | − |
| 71672 | T58002 | 4.77 | 4.16 | 2.13 | 1.15 | 2.23 | − | − | − | + |
| 565779 | AA135870 | 31.62 | 13.68 | 30.14 | 2.31 | 1.05 | − | + | − | − |
| 361323 | AA017544 | 4.53 | 6.49 | 1.70 | 0.70 | 2.66 | − | − | + | − |
| 111389 | T84308 | 2.59 | 2.15 | 1.21 | 1.20 | 2.14 | − | − | − | + |
| 731337 | AA416782 | 0.75 | 0.75 | 0.32 | 1.01 | 2.32 | − | − | − | + |
| 455271 | AA677574 | 1.64 | 1.62 | 0.71 | 1.01 | 2.30 | − | − | + | − |
| 488301 | AA085748 | 0.97 | 1.24 | 0.38 | 0.78 | 2.52 | − | − | + | − |
| 511107 | AA088371 | 7.14 | 7.60 | 3.02 | 0.94 | 2.36 | − | − | − | + |
| 626462 | AA188999 | 5.86 | 3.92 | 3.24 | 1.49 | 1.81 | − | − | − | + |
| 897906 | AA598652 | 10.34 | 13.66 | 4.08 | 0.76 | 2.54 | − | − | − | + |
| 810408 | AA457092 | 9.85 | 10.74 | 4.18 | 0.92 | 2.36 | − | − | − | + |
| 22600 | T87235 | 0.36 | 0.18 | 0.28 | 1.99 | 1.29 | + | − | − | − |
| 343688 | W69160 | 6.94 | 5.30 | 3.54 | 1.31 | 1.96 | − | − | + | − |
| 461761 | AA682399 | 8.65 | 14.46 | 3.24 | 0.60 | 2.67 | − | − | − | + |
| 784285 | AA447503 | 3.99 | 2.02 | 3.12 | 1.98 | 1.28 | − | + | − | − |
| 845780 | AI791127 | 5.95 | 4.75 | 2.97 | 1.25 | 2.00 | − | − | − | + |
| 266500 | N22687 | 7.14 | 7.39 | 3.12 | 0.97 | 2.29 | − | − | − | + |
| 119851 | T94611 | 6.52 | 3.28 | 5.16 | 1.98 | 1.26 | − | + | − | − |
| 809824 | AA455519 | 2.66 | 3.75 | 1.05 | 0.71 | 2.54 | − | − | − | + |
| 782513 | AA432030 | 5.78 | 4.46 | 2.98 | 1.29 | 1.94 | − | − | + | − |
| 648046 | AA206914 | 15.56 | 14.08 | 7.32 | 1.11 | 2.13 | − | − | − | + |
| 704076 | AA279172 | 5.70 | 2.95 | 4.40 | 1.93 | 1.30 | − | + | − | − |
| 855236 | AA782292 | 2.75 | 2.53 | 1.30 | 1.08 | 2.12 | − | − | + | − |
| 627053 | AA190997 | 11.04 | 13.42 | 4.64 | 0.82 | 2.38 | − | − | + | − |
| 738970 | AA421769 | 15.96 | 26.86 | 6.13 | 0.59 | 2.61 | − | − | + | − |
| 757191 | AA443966 | 6.08 | 6.70 | 2.66 | 0.91 | 2.29 | − | − | + | − |
| 740780 | AA477283 | 4.71 | 4.39 | 2.24 | 1.07 | 2.10 | − | − | + | − |
| 427812 | AA001614 | 9.94 | 12.03 | 4.27 | 0.83 | 2.33 | − | − | + | − |
| 1613940 | AI000294 | 3.48 | 3.17 | 1.70 | 1.10 | 2.05 | − | − | − | + |
| 361943 | AA001444 | 2.86 | 1.43 | 2.52 | 2.01 | 1.14 | + | − | − | − |
| 840698 | AA486370 | 7.45 | 7.83 | 3.42 | 0.95 | 2.18 | − | − | − | + |
| 220069 | H85437 | 4.81 | 8.77 | 1.92 | 0.55 | 2.50 | − | − | − | + |
| 1604717 | AA988630 | 13.38 | 13.90 | 6.58 | 0.96 | 2.03 | − | − | + | − |
| 41287 | R56613 | 0.72 | 0.52 | 0.45 | 1.38 | 1.59 | − | − | + | − |
| 460487 | AA677706 | 24.08 | 105.26 | 8.92 | 0.23 | 2.70 | − | − | + | + |
| 151477 | H02837 | 2.44 | 2.74 | 1.21 | 0.89 | 2.01 | − | − | + | − |
| 362080 | AA001434 | 3.81 | 1.72 | 5.56 | 2.21 | 0.69 | − | + | − | − |
| 289832 | N63172 | 6.67 | 4.11 | 5.46 | 1.62 | 1.22 | + | − | − | − |
| 767784 | AA418670 | 8.86 | 14.76 | 4.05 | 0.60 | 2.19 | − | − | + | − |
| 122428 | T99280 | 2.03 | 1.78 | 1.29 | 1.14 | 1.57 | − | − | + | − |
| 753794 | AA406115 | 1.65 | 0.94 | 1.78 | 1.75 | 0.93 | + | − | − | − |
| 563444 | AA112660 | 6.23 | 10.09 | 3.10 | 0.62 | 2.01 | − | − | − | + |
| 740941 | AA478298 | 12.33 | 29.84 | 6.23 | 0.41 | 1.98 | − | − | + | − |
| 462953 | AA682423 | 3.21 | 5.03 | 1.90 | 0.64 | 1.69 | − | − | + | − |
| 1492202 | AA875913 | 2.60 | 1.98 | 2.70 | 1.31 | 0.96 | + | − | − | − |

TABLE 1-4

| | GenBank | Aver. of | Aver. of | Aver. | Tumor | Tumor | T/N | T/N | T/N | T/B | T/B | T/B |
|---|---|---|---|---|---|---|---|---|---|---|---|---|
| Clone | Acc. No. | Tumors | Normals | of BPH | /Normal | /BPH | 10X, >1/5 | 5X, >3/5 | 3X, >5/5 | 10X, >1/5 | 5X, >3/5 | 3X, >5/5 |
| 788180 | AA453562 | 25.18 | 0.81 | 0.50 | 31.24 | 50.69 | + | + | + | + | + | + |
| 646037 | AA196979 | 24.42 | 1.02 | 0.75 | 23.91 | 32.70 | + | + | + | + | + | + |
| 1034473 | AA779728 | 33.61 | 1.51 | 1.35 | 22.27 | 24.97 | + | + | | + | | |
| 281003 | N50880 | 128.77 | 6.30 | 4.90 | 20.43 | 26.26 | + | + | + | + | + | + |
| 308682 | W25202 | 8.99 | 0.87 | 0.27 | 10.37 | 32.91 | + | + | + | + | + | + |
| 281039 | N47717 | 14.05 | 0.94 | 0.90 | 14.99 | 15.57 | + | | | + | | |
| 284701 | N64840 | 33.05 | 7.05 | 1.52 | 4.68 | 21.68 | | | | + | + | + |
| 823940 | AA490213 | 22.39 | 1.32 | 2.83 | 16.99 | 7.91 | + | + | + | + | | |
| 42415 | R60981 | 6.86 | 0.51 | 0.72 | 13.52 | 9.49 | + | + | | + | | |
| 46166 | H09076 | 13.13 | 0.93 | 1.71 | 14.12 | 7.68 | + | + | | + | | |
| 784168 | AA432103 | 53.04 | 7.37 | 3.83 | 7.20 | 13.84 | | + | | + | | + |
| 773479 | AA427899 | 12.32 | 0.65 | 5.99 | 18.85 | 2.05 | + | + | + | | | |
| 222157 | H85806 | 8.80 | 1.02 | 0.72 | 8.67 | 12.14 | + | + | + | + | | + |
| 486035 | AA043109 | 24.75 | 3.47 | 1.86 | 7.13 | 13.32 | + | + | + | + | + | + |
| 450049 | AA703396 | 18.37 | 1.83 | 1.80 | 10.05 | 10.21 | + | + | + | + | + | + |
| 66336 | T66832 | 1.67 | 0.63 | 0.10 | 2.63 | 16.70 | | | | + | | |
| 35058 | R45192 | 15.59 | 1.89 | 1.44 | 8.24 | 10.81 | + | + | + | + | | + |
| 26736 | R39891 | 9.52 | 3.36 | 0.62 | 2.83 | 15.37 | | | | + | | + |
| 593185 | AA159714 | 28.06 | 5.61 | 2.29 | 5.00 | 12.23 | + | | | + | | + |
| 32160 | R43432 | 26.80 | 3.59 | 2.77 | 7.47 | 9.69 | | + | + | + | + | + |
| 814053 | AA465495 | 14.66 | 1.70 | 1.75 | 8.61 | 8.38 | + | + | | + | | |
| 292515 | N68465 | 16.22 | 2.86 | 1.56 | 5.67 | 10.38 | | | + | + | + | + |
| 730739 | AA436097 | 36.49 | 7.10 | 3.45 | 5.14 | 10.57 | + | | + | + | + | + |
| 282089 | N48259 | 11.18 | 1.51 | 1.41 | 7.41 | 7.92 | + | | | + | | |
| 283312 | N54763 | 63.94 | 6.63 | 12.14 | 9.65 | 5.27 | + | + | + | | | |
| 291255 | N72215 | 15.20 | 1.35 | 4.65 | 11.29 | 3.27 | + | + | + | | | |
| 1048810 | AA621342 | 20.15 | 3.04 | 2.59 | 6.63 | 7.78 | + | + | + | + | | + |
| 197525 | H52119 | 9.52 | 1.85 | 1.06 | 5.16 | 9.01 | | | | + | | + |
| 67401 | T49326 | 1.47 | 0.20 | 0.23 | 7.28 | 6.27 | + | | | + | | |
| 490178 | AA121266 | 24.55 | 5.63 | 2.73 | 4.36 | 8.99 | + | | | + | | + |
| 1556526 | AA935560 | 0.88 | 0.15 | 0.12 | 5.96 | 7.33 | + | | | + | | |
| 746152 | AA419486 | 9.35 | 2.23 | 1.07 | 4.18 | 8.70 | | | | + | | |
| 1034738 | AA780190 | 22.40 | 3.90 | 3.28 | 5.74 | 6.82 | | + | + | | + | + |
| 259374 | N31952 | 10.51 | 2.33 | 1.31 | 4.50 | 8.04 | | | | + | | |
| 757435 | AA437224 | 365.78 | 59.86 | 58.35 | 6.11 | 6.27 | + | | + | + | | + |
| 754280 | AA479284 | 23.83 | 3.20 | 4.92 | 7.46 | 4.85 | + | + | + | | | |
| 877835 | AA625634 | 23.06 | 4.34 | 3.34 | 5.32 | 6.90 | | + | + | | | + |
| 712950 | AA282272 | 11.53 | 3.21 | 1.34 | 3.59 | 8.58 | + | | | + | | |
| 50772 | H16803 | 18.10 | 6.74 | 1.91 | 2.68 | 9.47 | | | | + | | + |
| 1636908 | AI000271 | 6.58 | 4.38 | 0.63 | 1.50 | 10.44 | | | | + | + | + |
| 432194 | AA679414 | 10.34 | 1.11 | 4.04 | 9.35 | 2.56 | + | + | + | | | |
| 488964 | AA047260 | 7.77 | 2.13 | 0.96 | 3.64 | 8.08 | + | | | + | | |
| 1412481 | AA845156 | 8.41 | 3.50 | 0.90 | 2.40 | 9.31 | + | | | + | | |
| 283173 | N45236 | 17.85 | 3.97 | 2.47 | 4.49 | 7.21 | + | | | + | | |
| 811867 | AA454963 | 24.57 | 2.97 | 7.43 | 8.28 | 3.30 | + | + | + | | | |
| 26519 | R20669 | 36.05 | 19.25 | 3.76 | 1.87 | 9.59 | | | | + | + | + |
| 687297 | AA235224 | 12.18 | 3.12 | 1.63 | 3.90 | 7.48 | | | | | + | + |
| 666334 | AA232249 | 1.64 | 1.54 | 0.16 | 1.07 | 10.14 | | | | + | + | + |
| 1468260 | AA884926 | 4.53 | 1.13 | 0.65 | 4.03 | 6.96 | | | | + | | |
| 258300 | N30680 | 18.15 | 4.53 | 2.65 | 4.01 | 6.86 | | | | + | | |
| 971279 | AA682905 | 54.34 | 6.63 | 22.20 | 8.20 | 2.45 | | + | + | | | |
| 1610448 | AA991856 | 135.55 | 57.06 | 16.44 | 2.38 | 8.25 | | | | + | | + |
| 461522 | AA705237 | 1.21 | 0.65 | 0.14 | 1.87 | 8.74 | | | | + | | |
| 868282 | AA633957 | 8.99 | 2.01 | 1.47 | 4.48 | 6.12 | | | + | | + | + |
| 280217 | N47941 | 20.52 | 5.86 | 2.89 | 3.50 | 7.09 | + | | | + | | |
| 240694 | H78135 | 4.53 | 0.90 | 0.83 | 5.01 | 5.47 | | | + | | | + |
| 321706 | W35398 | 7.80 | 1.86 | 1.25 | 4.20 | 6.23 | | | | + | | |
| 770066 | AA430545 | 11.43 | 2.60 | 1.93 | 4.40 | 5.94 | + | | | + | | |
| 811603 | AI732784 | 19.62 | 2.74 | 6.22 | 7.16 | 3.15 | | + | + | | | |
| 51773 | H23211 | 11.48 | 2.40 | 2.09 | 4.77 | 5.50 | | | + | | | + |
| 66317 | T66815 | 5.50 | 1.76 | 0.78 | 3.13 | 7.06 | | | | + | | |
| 35626 | R45292 | 9.42 | 1.09 | 6.51 | 8.67 | 1.45 | + | + | + | | | |
| 450307 | AA682851 | 9.39 | 1.62 | 2.25 | 5.80 | 4.17 | + | + | | | | |
| 447715 | AA702781 | 14.83 | 7.21 | 1.88 | 2.06 | 7.90 | | | | + | | |
| 731290 | AA416843 | 11.60 | 4.56 | 1.57 | 2.54 | 7.40 | | | | + | | |
| 825857 | AA504783 | 4.27 | 2.51 | 0.52 | 1.70 | 8.20 | | | | | + | + |
| 565235 | AA136125 | 35.83 | 12.59 | 5.15 | 2.85 | 6.96 | | | | + | | |
| 756600 | AA481464 | 47.06 | 7.13 | 14.79 | 6.60 | 3.18 | | + | + | | | |
| 293635 | N63807 | 26.47 | 3.44 | 12.94 | 7.69 | 2.05 | + | + | + | | | |
| 757392 | AA426110 | 0.86 | 0.37 | 0.12 | 2.32 | 7.41 | | | | | | + |
| 595695 | AA173189 | 3.58 | 2.33 | 0.44 | 1.54 | 8.16 | | | | | + | + |
| 151055 | H02230 | 27.32 | 7.51 | 4.62 | 3.64 | 5.92 | | | | + | | |
| 838366 | AA457172 | 11.15 | 1.82 | 3.27 | 6.12 | 3.41 | | + | + | | | |
| 460279 | AA677629 | 0.87 | 0.46 | 0.11 | 1.87 | 7.58 | | | | + | + | |
| 595297 | AA173598 | 3.07 | 2.45 | 0.38 | 1.25 | 8.17 | | | | + | + | + |
| 1631713 | AI025015 | 5.68 | 0.93 | 1.77 | 6.13 | 3.21 | | + | + | | | |
| 564801 | AA129551 | 12.35 | 3.96 | 1.99 | 3.12 | 6.21 | | | | + | | |
| 293651 | N69653 | 13.35 | 1.84 | 6.56 | 7.26 | 2.04 | + | + | + | | | |
| 745347 | AA625666 | 2.15 | 0.29 | 1.14 | 7.39 | 1.88 | | + | + | | | |
| 772304 | AA404486 | 113.14 | 18.10 | 38.24 | 6.25 | 2.96 | | + | + | | | |
| 262053 | N25030 | 10.75 | 5.15 | 1.52 | 2.09 | 7.08 | | | | | | + |
| 81331 | T60075 | 2.84 | 0.78 | 0.51 | 3.63 | 5.53 | + | | | + | | |
| 840894 | AA482340 | 34.81 | 11.87 | 5.66 | 2.93 | 6.15 | | | | | + | |
| 839081 | AA488732 | 16.60 | 5.37 | 2.78 | 3.09 | 5.98 | | | | + | | |
| 796366 | AA459761 | 34.94 | 17.03 | 5.05 | 2.05 | 6.92 | | | | + | | + |
| 359661 | AA011095 | 26.24 | 8.79 | 4.39 | 2.99 | 5.97 | | | | | | + |
| 50188 | H17943 | 10.75 | 2.29 | 2.53 | 4.69 | 4.25 | | | + | | | + |
| 84955 | T74846 | 5.26 | 1.01 | 1.43 | 5.21 | 3.69 | | + | | | | |
| 159608 | H16152 | 14.43 | 4.14 | 2.69 | 3.49 | 5.36 | | | | + | | |
| 1390584 | AA843592 | 6.54 | 1.91 | 1.21 | 3.42 | 5.41 | | | | + | | |
| 825736 | AA504842 | 0.92 | 0.60 | 0.13 | 1.54 | 7.26 | | | | | + | |
| 22161 | T66154 | 20.24 | 13.40 | 2.80 | 1.51 | 7.24 | | | | | + | + |
| 787938 | AA452278 | 5.09 | 1.27 | 1.08 | 4.01 | 4.71 | | | | | + | |
| 809603 | AA442991 | 49.03 | 6.94 | 29.52 | 7.06 | 1.66 | + | + | + | | | |
| 826109 | AA521327 | 0.75 | 0.54 | 0.10 | 1.38 | 7.27 | | | | + | | + |
| 884606 | AA630006 | 33.46 | 13.42 | 5.43 | 2.49 | 6.16 | | | | + | | |
| 825822 | AA491370 | 14.07 | 9.48 | 1.97 | 1.48 | 7.16 | | | | + | | + |
| 531028 | AA070495 | 21.14 | 3.89 | 6.62 | 5.44 | 3.20 | | + | + | | | |
| 814062 | AA465338 | 0.81 | 0.47 | 0.12 | 1.72 | 6.83 | | | | + | + | |
| 826103 | A521416 | 1.31 | 0.82 | 0.19 | 1.61 | 6.93 | | | | | + | |
| 461670 | AA779919 | 8.90 | 1.72 | 2.71 | 5.19 | 3.28 | | | + | | | |
| 415700 | W85697 | 3.36 | 0.61 | 1.15 | 5.53 | 2.93 | | + | | | | |
| 815163 | AA481144 | 1.20 | 0.57 | 0.19 | 2.09 | 6.36 | | | | | + | |
| 810558 | AA464568 | 14.49 | 3.00 | 4.01 | 4.83 | 3.61 | | | + | | | |
| 810220 | AA464689 | 2.17 | 0.39 | 0.77 | 5.60 | 2.82 | | + | | | | |
| 359781 | AA010932 | 7.82 | 2.50 | 1.49 | 3.13 | 5.26 | | | | | | + |
| 795265 | AA451807 | 4.58 | 1.26 | 0.97 | 3.63 | 4.74 | | | | + | | |
| 840306 | AA485333 | 2.49 | 1.26 | 0.39 | 1.97 | 6.38 | | | | | + | |
| 824723 | AA488986 | 14.11 | 4.37 | 2.84 | 3.23 | 4.96 | | | | | | + |
| 186918 | H43317 | 3.04 | 1.10 | 0.56 | 2.76 | 5.38 | | | | + | | |
| 191904 | H38880 | 7.67 | 1.44 | 2.75 | 5.32 | 2.79 | | + | + | | | |
| 771317 | AA476234 | 9.44 | 1.78 | 3.46 | 5.31 | 2.73 | | | + | | | |
| 826072 | AA521394 | 1.20 | 0.83 | 0.18 | 1.44 | 6.59 | | | | + | + | |
| 30428 | R42061 | 4.29 | 0.99 | 1.16 | 4.35 | 3.68 | + | | | + | | |
| 1056172 | AA620995 | 10.33 | 4.78 | 1.77 | 2.16 | 5.84 | | | | + | | |
| 251806 | H96647 | 2.94 | 1.83 | 0.46 | 1.60 | 6.39 | | | | | | + |
| 305538 | N89861 | 11.44 | 3.31 | 2.54 | 3.46 | 4.50 | | | | | + | |
| 52933 | H29315 | 3.59 | 1.99 | 0.58 | 1.80 | 6.14 | | | | | | + |
| 824510 | AA490522 | 1.38 | 1.09 | 0.21 | 1.27 | 6.65 | | | | | + | |
| 859228 | AA666366 | 85.06 | 37.74 | 15.20 | 2.25 | 5.60 | | | | | | + |
| 362755 | AA018675 | 16.82 | 3.11 | 6.91 | 5.41 | 2.43 | | | + | | | |
| 253132 | H89036 | 1.40 | 1.21 | 0.21 | 1.16 | 6.66 | | | | | | + |
| 233419 | H77706 | 5.55 | 1.14 | 1.90 | 4.87 | 2.93 | | + | | | | |
| 1592006 | AA953229 | 5.30 | 1.51 | 1.24 | 3.52 | 4.28 | | | + | | | + |
| 950778 | AA608636 | 3.00 | 1.87 | 0.48 | 1.60 | 6.18 | | | | + | | + |
| 951313 | AA620591 | 6.84 | 6.68 | 1.01 | 1.03 | 6.75 | | | | + | | |
| 771048 | AA427395 | 10.54 | 2.90 | 2.58 | 3.63 | 4.08 | | | | | | + |
| 840944 | AA486533 | 79.86 | 42.51 | 13.69 | 1.88 | 5.83 | | | | + | | |
| 268211 | N36327 | 3.43 | 0.70 | 1.23 | 4.93 | 2.78 | + | | | | | |
| 840606 | AA487914 | 15.85 | 7.18 | 2.88 | 2.21 | 5.50 | | | | | | + |
| 796161 | AA461092 | 30.31 | 5.27 | 15.52 | 5.75 | 1.95 | | + | + | | | |
| 811582 | AA458516 | 10.13 | 5.34 | 1.75 | 1.90 | 5.78 | | | | | | + |
| 449112 | AA777488 | 42.54 | 7.72 | 19.71 | 5.51 | 2.16 | | + | + | | | |
| 150262 | H01129 | 13.66 | 3.31 | 3.87 | 4.12 | 3.53 | | | + | | | |
| 788655 | AA449837 | 7.66 | 2.78 | 1.58 | 2.76 | 4.86 | | | | | | + |
| 509731 | AA045699 | 39.41 | 23.94 | 6.63 | 1.65 | 5.95 | | | | | | + |
| 565863 | AA136386 | 3.93 | 2.97 | 0.63 | 1.32 | 6.27 | | | | + | | |
| 509887 | AA054701 | 39.47 | 7.39 | 17.54 | 5.34 | 2.25 | | + | + | | | |
| 796646 | AA461467 | 6.21 | 4.35 | 1.01 | 1.43 | 6.16 | | | | + | | + |
| 814235 | AA465598 | 12.37 | 6.41 | 2.19 | 1.93 | 5.65 | | | | | + | |
| 53022 | R15880 | 1.34 | 0.70 | 0.24 | 1.90 | 5.63 | | | | | | + |
| 377246 | AA055306 | 12.60 | 3.02 | 3.78 | 4.17 | 3.34 | + | | | | | |
| 814662 | AA481045 | 1.12 | 0.67 | 0.19 | 1.67 | 5.84 | | | | | + | |
| 51700 | H22853 | 3.66 | 1.21 | 0.82 | 3.02 | 4.47 | | | | | | + |
| 80050 | T63893 | 8.15 | 3.84 | 1.52 | 2.12 | 5.35 | | | | + | | |
| 594994 | AA172372 | 14.61 | 10.98 | 2.38 | 1.33 | 6.14 | | | | | | + |
| 795907 | AA460346 | 5.39 | 1.25 | 1.71 | 4.31 | 3.15 | + | | | | | |
| 824126 | AA490611 | 0.59 | 0.37 | 0.10 | 1.58 | 5.87 | | | | | + | |
| 511952 | AA100674 | 225.66 | 92.92 | 45.17 | 2.43 | 5.00 | | | | + | | |
| 823615 | AA496957 | 9.40 | 3.54 | 1.97 | 2.65 | 4.76 | | | | + | | |
| 784130 | AA432075 | 17.23 | 6.93 | 3.50 | 2.49 | 4.93 | | | | | | + |
| 1535596 | AA918370 | 401.67 | 285.67 | 66.91 | 1.41 | 6.00 | | | | | | + |
| 1605407 | AA988746 | 9.00 | 4.61 | 1.65 | 1.95 | 5.45 | | | | + | | |
| 1606837 | AA996028 | 23.76 | 9.09 | 4.98 | 2.61 | 4.77 | | | | | + | |
| 796239 | AA460675 | 26.07 | 23.22 | 4.18 | 1.12 | 6.24 | | | | + | + | |
| 49302 | H15675 | 4.88 | 0.86 | 2.90 | 5.68 | 1.68 | + | | | | | |
| 754358 | AA436142 | 12.77 | 7.46 | 2.26 | 1.71 | 5.64 | | | | | + | |
| 207029 | R98842 | 81.88 | 14.79 | 45.41 | 5.54 | 1.80 | | + | + | | | |
| 838285 | AA458747 | 1.34 | 1.36 | 0.21 | 0.99 | 6.35 | | | | | | + |
| 1636108 | AI015679 | 3.58 | 1.45 | 0.74 | 2.47 | 4.85 | | | | | | + |
| 796754 | AA460719 | 0.66 | 0.83 | 0.10 | 0.80 | 6.50 | | | | | + | + |
| 771053 | AA430718 | 16.14 | 6.63 | 3.34 | 2.43 | 4.83 | | | | | | + |
| 289071 | N63609 | 37.48 | 15.32 | 7.78 | 2.45 | 4.82 | | | | + | | |
| 1637282 | AI005515 | 4.01 | 2.58 | 0.70 | 1.55 | 5.71 | | | | + | | |
| 263697 | H99672 | 4.23 | 0.93 | 1.58 | 4.57 | 2.68 | | + | | | | |
| 586780 | AA130677 | 1.92 | 1.75 | 0.31 | 1.10 | 6.12 | | | | + | | + |
| 416374 | W86202 | 4.49 | 2.66 | 0.81 | 1.69 | 5.52 | | | | + | | |
| 375682 | AA032221 | 31.04 | 12.66 | 6.60 | 2.45 | 4.70 | | | | | | + |
| 853968 | AI791173 | 12.10 | 2.86 | 4.21 | 4.23 | 2.87 | | + | | | | |
| 287728 | N75893 | 2.76 | 0.70 | 0.89 | 3.97 | 3.10 | + | | | | | |
| 842927 | AA486445 | 4.24 | 3.59 | 0.72 | 1.18 | 5.88 | | | | + | + | |
| 878281 | AA670286 | 6.80 | 3.30 | 1.37 | 2.06 | 4.97 | | | | | + | |
| 359701 | AA011176 | 5.21 | 2.32 | 1.09 | 2.25 | 4.78 | | | | | | + |
| 897807 | AA598531 | 5.08 | 2.77 | 0.98 | 1.83 | 5.16 | | | | | + | |
| 855749 | AA663983 | 50.50 | 12.03 | 18.24 | 4.20 | 2.77 | | | + | | | |
| 264895 | N21170 | 7.86 | 1.63 | 3.70 | 4.82 | 2.12 | | | + | | | |
| 825207 | AA504120 | 11.96 | 2.92 | 4.19 | 4.09 | 2.86 | | | + | | | |
| 461476 | AA705041 | 0.78 | 0.61 | 0.14 | 1.28 | 5.64 | | | | | + | |
| 449275 | AA777705 | 3.81 | 0.83 | 1.64 | 4.58 | 2.32 | + | | | | | |
| 755239 | AA422139 | 17.93 | 9.29 | 3.62 | 1.93 | 4.95 | | | | | | + |
| 259973 | N47829 | 1.66 | 1.95 | 0.28 | 0.85 | 5.99 | | | | | + | + |
| 290370 | N62301 | 0.62 | 0.48 | 0.11 | 1.30 | 5.53 | | | | | | + |
| 773287 | AA425534 | 15.94 | 8.17 | 3.27 | 1.95 | 4.87 | | | | | | + |
| 1472689 | AA873159 | 5.15 | 1.21 | 2.03 | 4.26 | 2.53 | + | | | | | |
| 252314 | H87175 | 0.69 | 0.62 | 0.12 | 1.11 | 5.66 | | | | | | + |
| 454317 | AA677165 | 20.56 | 33.95 | 3.34 | 0.61 | 6.16 | | | | + | | |
| 1585549 | AA976714 | 6.44 | 3.55 | 1.31 | 1.81 | 4.92 | | | | | | + |
| 491764 | AA150502 | 0.81 | 0.59 | 0.15 | 1.36 | 5.37 | | | | | | + |
| 139771 | R62352 | 7.72 | 6.17 | 1.42 | 1.25 | 5.44 | | | | + | | |
| 462097 | AA705376 | 8.32 | 6.33 | 1.55 | 1.31 | 5.38 | | | | | + | |
| 279482 | N48804 | 8.66 | 3.25 | 2.16 | 2.67 | 4.00 | | | | + | | |
| 824122 | AA490609 | 4.64 | 2.75 | 0.94 | 1.69 | 4.95 | | | | | + | |
| 1031583 | AA609323 | 38.01 | 9.06 | 15.59 | 4.20 | 2.44 | + | | | | | |
| 250869 | N23605 | 123.92 | 32.54 | 44.10 | 3.81 | 2.81 | + | | | | | |
| 896914 | AA779391 | 18.03 | 7.40 | 4.35 | 2.44 | 4.14 | | | | | + | |
| 814306 | AA459100 | 18.28 | 10.68 | 3.77 | 1.71 | 4.85 | | | | | | + |
| 77915 | T61271 | 14.99 | 17.48 | 2.63 | 0.86 | 5.70 | | | | + | | |
| 260116 | N32044 | 0.88 | 0.86 | 0.16 | 1.03 | 5.52 | | | | | | + |
| 210405 | H65395 | 34.02 | 7.84 | 15.49 | 4.34 | 2.20 | | | + | | | |
| 703943 | AA278473 | 4.77 | 2.81 | 0.99 | 1.70 | 4.82 | | | | | | + |
| 824267 | AA491265 | 0.74 | 0.41 | 0.16 | 1.81 | 4.68 | | | | | | + |
| 813973 | AA455689 | 3.24 | 0.88 | 1.18 | 3.70 | 2.75 | | | + | | | |
| 825297 | AA504536 | 0.96 | 0.65 | 0.19 | 1.48 | 4.97 | | | | | | + |
| 838807 | AA457681 | 1.90 | 2.58 | 0.33 | 0.74 | 5.71 | | | | | | + |
| 1607765 | AA989473 | 3.96 | 1.73 | 0.96 | 2.29 | 4.14 | | | | | | + |
| 86160 | T72336 | 2.10 | 0.74 | 0.59 | 2.82 | 3.58 | | | | | | + |
| 1456160 | AA862465 | 41.61 | 40.32 | 7.76 | 1.03 | 5.36 | | | | + | | |
| 897767 | AA598470 | 2.07 | 0.49 | 0.98 | 4.25 | 2.12 | | | + | | | |
| 1466631 | AA883679 | 1.21 | 0.71 | 0.26 | 1.71 | 4.63 | | | | | | + |
| 280763 | N50563 | 0.53 | 0.50 | 0.10 | 1.05 | 5.28 | | | | | | + |
| 768643 | AA430351 | 41.57 | 23.92 | 9.10 | 1.74 | 4.57 | | | | + | | |
| 610124 | AA171459 | 1.70 | 1.73 | 0.32 | 0.98 | 5.32 | | | | | | + |
| 1631849 | AI004315 | 25.01 | 12.24 | 5.91 | 2.04 | 4.24 | | | | | | + |
| 204111 | H55907 | 3.38 | 0.87 | 1.40 | 3.86 | 2.42 | | | + | | | |
| 491184 | AA137144 | 6.35 | 1.83 | 2.27 | 3.47 | 2.80 | + | | | | | |
| 824237 | AA491249 | 74.89 | 31.45 | 19.54 | 2.38 | 3.83 | | | | | | + |
| 24208 | R38412 | 7.49 | 3.51 | 1.85 | 2.13 | 4.05 | | | | | | + |
| 267419 | N24973 | 0.97 | 0.96 | 0.19 | 1.01 | 5.16 | | | | | | + |
| 731410 | AA412250 | 0.53 | 0.58 | 0.10 | 0.92 | 5.24 | | | | | | + |
| 869450 | AA680244 | 51.58 | 35.87 | 10.98 | 1.44 | 4.70 | | | | + | | |
| 124252 | R02439 | 6.06 | 4.74 | 1.25 | 1.28 | 4.85 | | | | + | | |
| 593658 | AA160080 | 1.08 | 1.02 | 0.21 | 1.07 | 5.04 | | | | | | + |
| 134969 | R31789 | 6.81 | 4.59 | 1.48 | 1.48 | 4.58 | | | | + | | |
| 430465 | AA680186 | 5.55 | 1.53 | 2.30 | 3.62 | 2.41 | + | | | | | |
| 796351 | AA459765 | 2.34 | 1.80 | 0.50 | 1.30 | 4.72 | | | | | | + |
| 549101 | AA083485 | 12.81 | 2.43 | 17.02 | 5.26 | 0.75 | | + | + | | | |
| 824376 | AA489696 | 0.49 | 0.34 | 0.11 | 1.42 | 4.58 | | | | | + | |
| 453599 | AA679569 | 4.23 | 2.23 | 1.03 | 1.89 | 4.10 | | | | | | + |
| 530185 | AA083671 | 8.46 | 7.83 | 1.73 | 1.08 | 4.88 | | | | | + | |
| 773248 | AA425773 | 21.90 | 19.94 | 4.51 | 1.10 | 4.86 | | | | | + | |
| 772416 | AA405597 | 1.03 | 1.35 | 0.20 | 0.76 | 5.19 | | | | | | + |
| 1456721 | AA864875 | 12.82 | 5.49 | 3.58 | 2.33 | 3.58 | | | | | | + |
| 50764 | H17363 | 0.43 | 0.23 | 0.11 | 1.89 | 4.00 | | | | | | + |
| 259462 | N29545 | 3.69 | 1.65 | 1.02 | 2.24 | 3.62 | | | | + | | |
| 366815 | AA029490 | 71.61 | 18.49 | 36.97 | 3.87 | 1.94 | + | | | | | |
| 731016 | AA421256 | 0.85 | 1.00 | 0.17 | 0.85 | 4.96 | | | | | | + |
| 197779 | R93515 | 8.61 | 6.61 | 1.91 | 1.30 | 4.50 | | | | + | | |
| 1519515 | AA902831 | 0.85 | 0.55 | 0.20 | 1.55 | 4.25 | | | | | | + |
| 511428 | AA126009 | 24.31 | 24.09 | 5.08 | 1.01 | 4.79 | | | | | | + |
| 757206 | AA443969 | 5.25 | 4.09 | 1.17 | 1.28 | 4.47 | | | | | | + |
| 264465 | N20522 | 0.77 | 0.84 | 0.16 | 0.91 | 4.79 | | | | | | + |
| 609052 | AA176607 | 0.87 | 0.87 | 0.19 | 0.99 | 4.63 | | | | | | + |
| 1637343 | AI015359 | 5.03 | 4.49 | 1.12 | 1.12 | 4.50 | | | | | | + |
| 186768 | H51914 | 9.43 | 5.75 | 2.39 | 1.64 | 3.95 | | | | + | | |
| 613237 | AA181767 | 1.07 | 0.78 | 0.25 | 1.37 | 4.22 | | | | | | + |
| 841302 | AA487213 | 7.41 | 3.64 | 2.09 | 2.04 | 3.54 | | | | + | | |
| 251517 | H96605 | 0.73 | 0.80 | 0.16 | 0.91 | 4.64 | | | | | | + |
| 855624 | AA664101 | 17.75 | 8.17 | 5.31 | 2.17 | 3.34 | | | | + | | |
| 841220 | AA486723 | 1.46 | 1.23 | 0.34 | 1.18 | 4.32 | | | | | | + |
| 1631194 | AA994757 | 21.83 | 14.39 | 5.47 | 1.52 | 3.99 | | | | | + | |
| 251435 | H97989 | 0.73 | 0.90 | 0.16 | 0.81 | 4.68 | | | | | | + |
| 767406 | AA417911 | 0.44 | 0.45 | 0.10 | 0.98 | 4.45 | | | | | | + |
| 745283 | AA625567 | 7.94 | 5.76 | 1.97 | 1.38 | 4.02 | | | | | | + |
| 815017 | AA465258 | 14.81 | 9.71 | 3.84 | 1.52 | 3.85 | | | | | | + |
| 346889 | W79834 | 0.89 | 0.21 | 0.79 | 4.21 | 1.12 | | + | | | | |
| 823895 | AA490490 | 1.57 | 1.72 | 0.36 | 0.91 | 4.40 | | | | | | + |
| 1553306 | AA934762 | 7.92 | 5.52 | 2.06 | 1.43 | 3.84 | | | | | | + |
| 361922 | AA001403 | 9.18 | 5.49 | 2.55 | 1.67 | 3.59 | | | | | | + |
| 290378 | N64508 | 17.10 | 17.02 | 4.07 | 1.00 | 4.20 | | | | | | + |
| 1602619 | AA988798 | 135.32 | 117.37 | 33.70 | 1.15 | 4.01 | | | | | | + |
| 815800 | AA485216 | 1.22 | 1.44 | 0.29 | 0.84 | 4.25 | | | | | + | |
| 377672 | AA056001 | 2.59 | 1.39 | 0.81 | 1.87 | 3.20 | | | | + | | |
| 256680 | H96392 | 1.72 | 1.47 | 0.44 | 1.17 | 3.88 | | | | | | + |
| 823578 | AA497118 | 0.56 | 0.53 | 0.14 | 1.06 | 3.98 | | | | | | + |
| 878152 | AA775431 | 4.20 | 3.49 | 1.10 | 1.20 | 3.82 | | | | | | + |
| 685922 | AA262559 | 3.59 | 3.70 | 0.90 | 0.97 | 3.99 | | | | | | + |
| 786525 | AA452113 | 1.46 | 0.42 | 1.00 | 3.46 | 1.46 | + | | | | | |
| 1594019 | AA988701 | 24.27 | 24.53 | 6.55 | 0.99 | 3.70 | | | | | | + |
| 282283 | N52675 | 0.84 | 1.00 | 0.22 | 0.84 | 3.85 | | | | | | + |
| 813271 | AA455938 | 0.96 | 1.12 | 0.25 | 0.85 | 3.79 | | | | | | + |
| 461336 | AA699770 | 3.42 | 4.00 | 0.91 | 0.85 | 3.74 | | | | | | + |
| 742581 | AA400247 | 1.20 | 1.08 | 0.36 | 1.11 | 3.31 | | | | | | + |
| 161456 | H25546 | 13.35 | 10.78 | 5.08 | 1.24 | 2.63 | | | | + | | |

TABLE 1-5

| | GenBank | Aver. | Aver. | Aver. | Prostate | T/N | |
|---|---|---|---|---|---|---|---|
| Clone | Acc No. | of | of BPH | of | Spec Exp | & T/BPH | Secreted |
| 282089 | N48259 | 1.51 | 1.41 | 11.18 | + | + | − |
| 646037 | AA196979 | 1.02 | 0.75 | 24.42 | + | + | + |
| 745283 | AA625567 | 5.76 | 1.97 | 7.94 | + | | − |
| 30428 | R42061 | 0.99 | 1.16 | 4.29 | | + | + |
| 35058 | R45192 | 1.89 | 1.44 | 15.59 | | + | + |
| 46166 | H09076 | 0.93 | 1.71 | 13.13 | | + | + |
| 67401 | T49326 | 0.20 | 0.23 | 1.47 | | + | + |
| 240694 | H78135 | 0.90 | 0.83 | 4.53 | | + | + |
| 280217 | N47941 | 5.86 | 2.89 | 20.52 | | + | + |
| 292515 | N68465 | 2.86 | 1.56 | 16.22 | | + | + |
| 593185 | AA159714 | 5.61 | 2.29 | 28.06 | | + | + |
| 730739 | AA436097 | 7.10 | 3.45 | 36.49 | | + | + |
| 1592006 | AA953229 | 1.51 | 1.24 | 5.30 | | + | + |
| 32160 | R43432 | 3.59 | 2.77 | 26.80 | | + | |
| 42415 | R60981 | 0.51 | 0.72 | 6.86 | | + | |
| 50188 | H17943 | 2.29 | 2.53 | 10.75 | | + | |
| 51773 | H23211 | 2.40 | 2.09 | 11.48 | | + | |
| 81331 | T60075 | 0.78 | 0.51 | 2.84 | | + | |
| 222157 | H85806 | 1.02 | 0.72 | 8.80 | | + | |
| 281003 | N50880 | 6.30 | 4.90 | 128.77 | | + | |
| 281039 | N47717 | 0.94 | 0.90 | 14.05 | | + | |
| 283173 | N45236 | 3.97 | 2.47 | 17.85 | | + | |
| 308682 | W25202 | 0.87 | 0.27 | 8.99 | | + | |
| 450049 | AA703396 | 1.83 | 1.80 | 18.37 | | + | |
| 486035 | AA043109 | 3.47 | 1.86 | 24.75 | | + | |
| 488964 | AA047260 | 2.13 | 0.96 | 7.77 | | + | |
| 490178 | AA121266 | 5.63 | 2.73 | 24.55 | | + | |
| 712950 | AA282272 | 3.21 | 1.34 | 11.53 | | + | |
| 757435 | AA437224 | 59.86 | 58.35 | 365.78 | | + | |
| 784168 | AA432103 | 7.37 | 3.83 | 53.04 | | + | |
| 788180 | AA453562 | 0.81 | 0.50 | 25.18 | | + | |
| 814053 | AA465495 | 1.70 | 1.75 | 14.66 | | + | |
| 823940 | AA490213 | 1.32 | 2.83 | 22.39 | | + | |
| 868282 | AA633957 | 2.01 | 1.47 | 8.99 | | + | |
| 877835 | AA625634 | 4.34 | 3.34 | 23.06 | | + | |
| 1034473 | AA779728 | 1.51 | 1.35 | 33.61 | | + | |
| 1034738 | AA780190 | 3.90 | 3.28 | 22.40 | | + | |
| 1048810 | AA621342 | 3.04 | 2.59 | 20.15 | | + | |
| 1412481 | AA845156 | 3.50 | 0.90 | 8.41 | | + | |
| 1556526 | AA935560 | 0.15 | 0.12 | 0.88 | | + | |
| 26519 | R20669 | 19.25 | 3.76 | 36.05 | | | + |
| 35626 | R45292 | 1.09 | 6.51 | 9.42 | | | + |
| 51700 | H22853 | 1.21 | 0.82 | 3.66 | | | + |
| 52933 | H29315 | 1.99 | 0.58 | 3.59 | | | + |
| 77915 | T61271 | 17.48 | 2.63 | 14.99 | | | + |
| 80050 | T63893 | 3.84 | 1.52 | 8.15 | | | + |
| 84955 | 174846 | 1.01 | 1.43 | 5.26 | | | + |
| 124252 | R02439 | 4.74 | 1.25 | 6.06 | | | + |
| 134969 | R31789 | 4.59 | 1.48 | 6.81 | | | + |
| 150262 | H01129 | 3.31 | 3.87 | 13.66 | | | + |
| 151055 | H02230 | 7.51 | 4.62 | 27.32 | | | + |
| 159608 | H16152 | 4.14 | 2.69 | 14.43 | | | + |
| 186768 | H51914 | 5.75 | 2.39 | 9.43 | | | + |
| 186918 | H43317 | 1.10 | 0.56 | 3.04 | | | + |
| 233419 | H77706 | 1.14 | 1.90 | 5.55 | | | + |
| 251806 | H96647 | 1.83 | 0.46 | 2.94 | | | + |
| 252314 | H87175 | 0.62 | 0.12 | 0.69 | | | + |
| 256680 | H96392 | 1.47 | 0.44 | 1.72 | | | + |
| 259462 | N29545 | 1.65 | 1.02 | 3.69 | | | + |
| 260116 | N32044 | 0.86 | 0.16 | 0.88 | | | + |
| 263697 | H99672 | 0.93 | 1.58 | 4.23 | | | + |
| 280763 | N50563 | 0.50 | 0.10 | 0.53 | | | + |
| 283312 | N54763 | 6.63 | 12.14 | 63.94 | | | + |
| 284701 | N64840 | 7.05 | 1.52 | 33.05 | | | + |
| 290370 | N62301 | 0.48 | 0.11 | 0.62 | | | + |
| 290378 | N64508 | 17.02 | 4.07 | 17.10 | | | + |
| 291255 | N72215 | 1.35 | 4.65 | 15.20 | | | + |
| 293635 | N63807 | 3.44 | 12.94 | 26.47 | | | + |
| 293651 | N69653 | 1.84 | 6.56 | 13.35 | | | + |
| 321706 | W35398 | 1.86 | 1.25 | 7.80 | | | + |
| 346889 | W79834 | 0.21 | 0.79 | 0.89 | | | + |
| 361922 | AA001403 | 5.49 | 2.55 | 9.18 | | | + |
| 375682 | AA032221 | 12.66 | 6.60 | 31.04 | | | + |
| 377672 | AA056001 | 1.39 | 0.81 | 2.59 | | | + |
| 416374 | W86202 | 2.66 | 0.81 | 4.49 | | | + |
| 430465 | AA680186 | 1.53 | 2.30 | 5.55 | | | + |
| 450307 | AA682851 | 1.62 | 2.25 | 9.39 | | | + |
| 454317 | AA677165 | 33.95 | 3.34 | 20.56 | | | + |
| 461336 | AA699770 | 4.00 | 0.91 | 3.42 | | | + |
| 461476 | AA705041 | 0.61 | 0.14 | 0.78 | | | + |
| 491184 | AA137144 | 1.83 | 2.27 | 6.35 | | | + |
| 491764 | AA150502 | 0.59 | 0.15 | 0.81 | | | + |
| 509731 | AA045699 | 23.94 | 6.63 | 39.41 | | | + |
| 509887 | AA054701 | 7.39 | 17.54 | 39.47 | | | + |
| 511428 | AA126009 | 24.09 | 5.08 | 24.31 | | | + |
| 511952 | AA100674 | 92.92 | 45.17 | 225.66 | | | + |
| 530185 | AA083671 | 7.83 | 1.73 | 8.46 | | | + |
| 564801 | AA129551 | 3.96 | 1.99 | 12.35 | | | + |
| 586780 | AA130677 | 1.75 | 0.31 | 1.92 | | | + |
| 593658 | AA160080 | 1.02 | 0.21 | 1.08 | | | + |
| 594994 | AA172372 | 10.98 | 2.38 | 14.61 | | | + |
| 595695 | AA173189 | 2.33 | 0.44 | 3.58 | | | + |
| 609052 | AA176607 | 0.87 | 0.19 | 0.87 | | | + |
| 610124 | AA171459 | 1.73 | 0.32 | 1.70 | | | + |
| 666334 | AA232249 | 1.54 | 0.16 | 1.64 | | | + |
| 731410 | AA412250 | 0.58 | 0.10 | 0.53 | | | + |
| 756600 | AA481464 | 7.13 | 14.79 | 47.06 | | | + |
| 757392 | AA426110 | 0.37 | 0.12 | 0.86 | | | + |
| 767406 | AA417911 | 0.45 | 0.10 | 0.44 | | | + |
| 768643 | AA430351 | 23.92 | 9.10 | 41.57 | | | + |
| 770066 | AA430545 | 2.60 | 1.93 | 11.43 | | | + |
| 773248 | AA425773 | 19.94 | 4.51 | 21.90 | | | + |
| 773479 | AA427899 | 0.65 | 5.99 | 12.32 | | | + |
| 784130 | AA432075 | 6.93 | 3.50 | 17.23 | | | + |
| 787938 | AA452278 | 1.27 | 1.08 | 5.09 | | | + |
| 788655 | AA449837 | 2.78 | 1.58 | 7.66 | | | + |
| 795265 | AA451807 | 1.26 | 0.97 | 4.58 | | | + |
| 796161 | AA461092 | 5.27 | 15.52 | 30.31 | | | + |
| 796239 | AA460675 | 23.22 | 4.18 | 26.07 | | | + |
| 811582 | AA458516 | 5.34 | 1.75 | 10.13 | | | + |
| 811603 | A1732784 | 2.74 | 6.22 | 19.62 | | | + |
| 811867 | AA454963 | 2.97 | 7.43 | 24.57 | | | + |
| 815163 | AA481144 | 0.57 | 0.19 | 1.20 | | | + |
| 823615 | AA496957 | 3.54 | 1.97 | 9.40 | | | + |
| 823895 | AA490490 | 1.72 | 0.36 | 1.57 | | | + |
| 824126 | AA490611 | 0.37 | 0.10 | 0.59 | | | + |
| 825822 | AA491370 | 9.48 | 1.97 | 14.07 | | | + |
| 826072 | AA521394 | 0.83 | 0.18 | 1.20 | | | + |
| 826109 | AA521327 | 0.54 | 0.10 | 0.75 | | | + |
| 838285 | AA458747 | 1.36 | 0.21 | 1.34 | | | + |
| 838807 | AA457681 | 2.58 | 0.33 | 1.90 | | | + |
| 839081 | AA488732 | 5.37 | 2.78 | 16.60 | | | + |
| 840894 | AA482340 | 11.87 | 5.66 | 34.81 | | | + |
| 855624 | AA664101 | 8.17 | 5.31 | 17.75 | | | + |
| 878152 | AA775431 | 3.49 | 1.10 | 4.20 | | | + |
| 884606 | AA630006 | 13.42 | 5.43 | 33.46 | | | + |
| 897767 | AA598470 | 0.49 | 0.98 | 2.07 | | | + |
| 951313 | AA620591 | 6.68 | 1.01 | 6.84 | | | + |
| 1031583 | AA609323 | 9.06 | 15.59 | 38.01 | | | + |
| 1056172 | AA620995 | 4.78 | 1.77 | 10.33 | | | + |
| 1456160 | AA862465 | 40.32 | 7.76 | 41.61 | | | + |
| 1472689 | AA873159 | 1.21 | 2.03 | 5.15 | | | + |
| 1535596 | AA918370 | 285.67 | 66.91 | 401.67 | | | + |
| 1610448 | AA991856 | 57.06 | 16.44 | 135.55 | | | + |
| 1631849 | A1004315 | 12.24 | 5.91 | 25.01 | | | + |

TABLE 2-1
T3NO vs BPH: Genes Upregulated 5 fold or higher in at least 1
tumor sample or 3 fold or higher in at least 2 tumor samples.

| | | | | | Fold | Fold | Fold | Fold | Fold | Fold | Fold | |
|---|---|---|---|---|---|---|---|---|---|---|---|---|
| | | Gen Bank | | | (Ave | (MPM | (MPM | (MPM | (MPM | (MPM | (MPM | # of |
| | IMAGE | Accession | | Ave | Tumor/ | 120/Ave | 121/Ave | 124/Ave | 122/Ave | 123/Ave | 125/Ave | Tumor |
| | Clone ID | Number | AveBPH | Tumor | AveBPH) | BPH) | BPH) | BPH) | BPH) | BPH) | BPH) | Samples |
| 1 | 788180 | AA453310 | 1.96 | 133.47 | 67.99 | 149.63 | 165.70 | 7.38 | 52.32 | 22.54 | 10.38 | 6 |
| 2 | 646037 | AA196979 | 2.38 | 114.00 | 47.98 | 65.24 | 24.62 | 77.47 | 31.06 | 53.59 | 35.90 | 6 |
| 3 | 284701 | N64840 | 2.41 | 73.10 | 30.37 | 31.27 | 50.07 | 34.87 | 5.26 | 41.24 | 19.50 | 6 |
| 4 | 450049 | AA703396 | 2.55 | 76.64 | 30.08 | 38.52 | 11.57 | 48.94 | 15.57 | 45.42 | 20.45 | 6 |
| 5 | 245768 | N55266 | 0.01 | 0.21 | 22.49 | 11.34 | 8.80 | 10.09 | 19.72 | 21.41 | 63.61 | 6 |
| 6 | 281003 | N50880 | 24.05 | 423.65 | 17.62 | 22.19 | 20.64 | 22.40 | 15.57 | 11.61 | 13.29 | 6 |
| 7 | 136605 | R35051 | 2.56 | 44.38 | 17.34 | 29.78 | 41.52 | 9.28 | 10.57 | 5.71 | 7.21 | 6 |
| 8 | 133130 | R28397 | 1.48 | 25.46 | 17.23 | 30.48 | 41.34 | 6.15 | 9.24 | 10.23 | 5.96 | 6 |
| 9 | 838668 | AA457235 | 3.44 | 48.10 | 13.39 | 11.55 | 50.28 | 3.85 | 4.31 | 3.32 | 7.01 | 6 |
| 10 | 179753 | H51549 | 0.01 | 0.12 | 11.81 | 9.28 | 11.84 | 11.99 | 10.01 | 17.09 | 10.68 | 6 |
| 11 | 1030855 | AA621761 | 1.24 | 12.57 | 10.16 | 7.99 | 15.61 | 11.93 | 8.14 | 4.27 | 15.03 | 6 |
| 12 | 51433 | H20747 | 2.07 | 19.56 | 9.43 | 6.78 | 6.32 | 8.58 | 7.12 | 8.55 | 19.24 | 6 |
| 13 | 306066 | N91003 | 0.80 | 6.94 | 8.70 | 13.54 | 16.10 | 7.85 | 4.72 | 3.65 | 6.31 | 6 |
| 14 | 195975 | R91398 | 0.50 | 3.83 | 7.67 | 5.89 | 6.60 | 8.91 | 5.51 | 12.71 | 6.41 | 6 |
| 15 | 21931 | T72543 | 0.29 | 1.97 | 6.87 | 3.24 | 10.05 | 6.80 | 7.18 | 5.75 | 8.20 | 6 |
| 16 | 292217 | N79180 | 0.06 | 0.42 | 6.73 | 3.94 | 10.11 | 7.14 | 10.04 | 4.07 | 5.11 | 6 |
| 17 | 825859 | AA504784 | 1.91 | 12.19 | 6.37 | 6.28 | 4.64 | 5.38 | 5.13 | 5.23 | 11.57 | 6 |
| 18 | 428816 | AA004881 | 0.10 | 0.60 | 6.03 | 6.54 | 8.44 | 8.60 | 4.79 | 4.07 | 3.73 | 6 |
| 19 | 195034 | R88764 | 0.02 | 0.14 | 5.84 | 3.19 | 6.01 | 6.21 | 7.48 | 3.67 | 8.48 | 6 |
| 20 | 359701 | AA011176 | 2.91 | 12.87 | 4.42 | 3.21 | 3.87 | 3.89 | 8.96 | 3.53 | 3.06 | 6 |
| 21 | 243641 | N49548 | 1.55 | 6.34 | 4.09 | 3.17 | 5.98 | 4.16 | 3.84 | 3.52 | 3.85 | 6 |
| 22 | 282035 | H98688 | 0.22 | 0.86 | 3.95 | 3.77 | 4.13 | 3.55 | 4.47 | 3.77 | 4.01 | 6 |
| 23 | 287528 | N62096 | 0.30 | 3.88 | 12.94 | 15.20 | 2.45 | 18.94 | 12.53 | 19.49 | 9.01 | 5 |
| 24 | 784168 | AA432103 | 15.97 | 194.72 | 12.19 | 9.62 | 26.16 | 14.54 | 17.70 | 1.22 | 3.92 | 5 |
| 25 | 259374 | N31952 | 1.29 | 13.52 | 10.47 | 7.91 | 24.39 | 10.24 | 5.27 | 13.26 | 1.75 | 5 |
| 26 | 210820 | H67712 | 0.16 | 1.82 | 9.61 | 2.75 | 9.06 | 3.94 | 3.08 | 33.98 | 4.85 | 5 |
| 27 | 193923 | R83407 | 17.59 | 158.73 | 9.02 | 10.87 | 18.00 | 7.87 | 13.10 | 1.22 | 3.27 | 5 |
| 28 | 37980 | R81372 | 0.09 | 0.74 | 8.39 | 9.46 | 28.73 | 3.79 | 3.02 | 4.81 | 0.54 | 5 |
| 29 | 1031599 | AA809485 | 0.27 | 2.07 | 7.65 | 5.69 | 10.96 | 6.72 | 12.64 | 2.83 | 7.08 | 5 |
| 30 | 86160 | T72336 | 1.71 | 11.25 | 6.59 | 7.39 | 5.16 | 4.03 | 15.83 | 2.66 | 4.49 | 5 |
| 31 | 288211 | N29986 | 0.57 | 3.71 | 6.48 | 8.59 | 3.88 | 13.77 | 2.12 | 4.36 | 6.18 | 5 |
| 32 | 482468 | AA699895 | 0.38 | 2.39 | 8.34 | 5.95 | 10.82 | 5.48 | 10.02 | 1.94 | 3.81 | 5 |
| 33 | 866276 | AA663160 | 3.26 | 20.49 | 6.28 | 1.91 | 3.57 | 4.27 | 14.38 | 9.64 | 3.94 | 5 |
| 34 | 811582 | AA454597 | 3.59 | 21.22 | 5.92 | 9.12 | 4.48 | 4.71 | 6.04 | 2.69 | 8.45 | 5 |
| 35 | 428436 | AA004412 | 0.53 | 3.12 | 5.83 | 1.13 | 4.65 | 4.73 | 5.71 | 11.11 | 7.68 | 5 |
| 36 | 130136 | R20813 | 0.04 | 0.20 | 5.62 | 7.23 | 6.80 | 5.30 | 5.38 | 6.96 | 2.08 | 5 |
| 37 | 347586 | W81432 | 0.04 | 0.24 | 5.62 | 2.99 | 4.97 | 4.10 | 6.44 | 3.72 | 11.48 | 5 |
| 38 | 795729 | AA460291 | 0.02 | 0.11 | 5.58 | 5.69 | 4.98 | 0.83 | 11.93 | 6.34 | 3.73 | 5 |
| 39 | 825785 | AA505117 | 1.56 | 8.57 | 5.51 | 3.24 | 6.98 | 2.95 | 6.31 | 9.01 | 4.56 | 5 |
| 40 | 49858 | H29290 | 0.33 | 1.80 | 5.39 | 2.31 | 3.63 | 6.60 | 3.61 | 13.09 | 3.10 | 5 |
| 41 | 417075 | W87823 | 0.83 | 4.38 | 5.30 | 2.79 | 3.52 | 3.68 | 4.21 | 8.09 | 9.54 | 5 |
| 42 | 840751 | AA486071 | 1.73 | 9.09 | 5.25 | 3.31 | 10.29 | 3.87 | 2.36 | 7.56 | 4.13 | 5 |
| 43 | 241821 | H93381 | 1.06 | 5.43 | 5.10 | 3.95 | 6.61 | 5.92 | 8.06 | 2.79 | 3.31 | 5 |
| 44 | 29063 | R40970 | 1.68 | 8.52 | 5.08 | 4.26 | 6.83 | 4.57 | 7.80 | 4.62 | 2.43 | 5 |
| 45 | 124128 | R01348 | 1.25 | 6.16 | 4.92 | 2.53 | 5.09 | 3.89 | 4.46 | 8.48 | 5.11 | 5 |
| 46 | 238821 | H85030 | 4.70 | 23.13 | 4.92 | 2.33 | 3.91 | 3.66 | 11.43 | 4.02 | 4.20 | 5 |
| 47 | 134719 | R28287 | 0.29 | 1.40 | 4.85 | 5.02 | 7.58 | 4.08 | 5.94 | 3.53 | 2.93 | 5 |
| 48 | 287745 | N82244 | 3.81 | 18.41 | 4.83 | 2.38 | 4.28 | 3.75 | 9.64 | 3.82 | 5.12 | 5 |
| 49 | 271141 | N34482 | 0.01 | 0.06 | 4.58 | 0.79 | 8.80 | 4.19 | 3.79 | 5.37 | 4.56 | 5 |
| 50 | 1240414 | AA788780 | 0.79 | 3.60 | 4.55 | 1.68 | 4.13 | 3.48 | 7.19 | 6.34 | 4.47 | 5 |
| 51 | 307249 | N93438 | 3.36 | 15.26 | 4.54 | 9.25 | 5.31 | 0.40 | 5.39 | 3.29 | 3.63 | 5 |
| 52 | 783849 | AA443688 | 0.13 | 0.59 | 4.48 | 4.82 | 5.19 | 6.45 | 4.67 | 1.71 | 4.04 | 5 |
| 53 | 897720 | AA598982 | 0.32 | 1.38 | 4.37 | 3.68 | 3.50 | 2.94 | 3.83 | 6.46 | 5.83 | 5 |
| 54 | 283070 | N51297 | 0.03 | 0.12 | 4.31 | 3.87 | 6.96 | 3.48 | 5.59 | 1.54 | 4.45 | 5 |
| 55 | 265005 | N21081 | 0.05 | 0.20 | 4.23 | 6.18 | 3.28 | 3.22 | 6.00 | 0.56 | 6.16 | 5 |
| 56 | 343987 | W70234 | 8.51 | 35.37 | 4.18 | 6.16 | 7.98 | 3.20 | 3.85 | 0.24 | 3.50 | 5 |
| 57 | 418855 | W87281 | 0.46 | 1.88 | 4.03 | 2.15 | 7.06 | 3.45 | 3.09 | 5.14 | 3.31 | 5 |
| 58 | 842848 | AA488281 | 1.15 | 4.62 | 4.02 | 3.49 | 4.63 | 2.64 | 4.44 | 5.85 | 3.05 | 5 |
| 59 | 40699 | R55882 | 0.05 | 0.18 | 3.70 | 3.25 | 3.93 | 4.50 | 3.68 | 4.50 | 2.31 | 5 |
| 60 | 359661 | AA011096 | 23.32 | 84.82 | 3.64 | 4.70 | 6.56 | 3.16 | 3.29 | 3.49 | 0.64 | 5 |
| 61 | 128230 | R06307 | 0.03 | 0.10 | 3.50 | 3.19 | 3.36 | 3.88 | 4.66 | 4.99 | 0.89 | 5 |
| 62 | 375882 | AA032221 | 15.66 | 45.43 | 2.90 | 3.03 | 3.29 | 3.51 | 3.03 | 3.10 | 1.45 | 5 |
| 63 | 1488260 | AA884926 | 0.65 | 22.36 | 34.16 | 18.89 | 1.69 | 59.34 | 105.24 | 0.80 | 19.00 | 4 |
| 64 | 743828 | AA634379 | 0.58 | 12.90 | 22.20 | 29.78 | 48.11 | 29.56 | 0.88 | 23.62 | 1.26 | 4 |
| 65 | 1390584 | AA843592 | 1.84 | 33.09 | 18.02 | 49.03 | 48.43 | 3.22 | 1.15 | 2.77 | 3.51 | 4 |
| 66 | 41647 | R52794 | 0.40 | 6.34 | 15.84 | 9.65 | 9.81 | 1.44 | 57.03 | 15.40 | 1.72 | 4 |
| 67 | 744944 | AA625890 | 1.90 | 22.42 | 11.81 | 2.76 | 4.13 | 1.86 | 8.52 | 48.21 | 5.35 | 4 |
| 68 | 1034473 | AA779728 | 0.13 | 1.36 | 10.50 | 22.49 | 23.74 | 3.29 | 9.26 | 2.32 | 1.91 | 4 |
| 69 | 487165 | AA045074 | 0.01 | 0.09 | 8.96 | 13.49 | 10.45 | 13.83 | 13.97 | 1.00 | 1.00 | 4 |
| 70 | 1458424 | AA882992 | 0.08 | 0.69 | 8.20 | 10.16 | 21.53 | 1.58 | 8.31 | 4.84 | 2.76 | 4 |
| 71 | 380155 | AA013260 | 0.01 | 0.11 | 8.02 | 10.07 | 0.73 | 2.20 | 24.39 | 4.57 | 6.15 | 4 |
| 72 | 144740 | R76229 | 0.19 | 1.27 | 6.55 | 7.58 | 10.38 | 6.62 | 10.46 | 1.64 | 2.63 | 4 |
| 73 | 24915 | R39068 | 0.93 | 5.97 | 6.42 | 3.07 | 2.31 | 4.37 | 2.85 | 10.25 | 15.67 | 4 |
| 74 | 593185 | AA165878 | 9.70 | 60.24 | 6.21 | 9.37 | 12.99 | 2.46 | 6.80 | 2.35 | 3.29 | 4 |
| 75 | 1056172 | AA620995 | 11.61 | 70.00 | 6.03 | 6.66 | 5.08 | 10.11 | 2.19 | 1.74 | 10.42 | 4 |
| 76 | 782383 | AA431407 | 0.95 | 5.55 | 5.84 | 8.95 | 11.77 | 5.12 | 5.02 | 1.51 | 2.67 | 4 |
| 77 | 1031113 | AA609914 | 0.01 | 0.07 | 5.65 | 16.58 | 0.83 | 6.44 | 4.81 | 2.05 | 3.19 | 4 |
| 78 | 481541 | AA705258 | 0.34 | 1.88 | 5.48 | 2.60 | 6.47 | 9.51 | 6.43 | 5.07 | 2.77 | 4 |
| 79 | 491706 | AA115259 | 0.75 | 3.90 | 5.23 | 3.93 | 6.17 | 7.31 | 9.02 | 2.29 | 2.67 | 4 |
| 80 | 787938 | AA452278 | 1.76 | 8.87 | 5.04 | 8.20 | 9.84 | 8.85 | 1.30 | 0.28 | 5.98 | 4 |
| 81 | 754034 | AA479058 | 0.02 | 0.10 | 5.01 | 5.74 | 6.67 | 0.33 | 1.52 | 6.64 | 9.17 | 4 |
| 82 | 970590 | AA683077 | 0.04 | 0.18 | 4.93 | 3.02 | 13.78 | 4.92 | 2.90 | 1.71 | 3.26 | 4 |
| 83 | 68767 | T53389 | 0.02 | 0.08 | 4.91 | 1.66 | 3.59 | 10.99 | 8.52 | 0.64 | 4.09 | 4 |
| 84 | 292515 | N68465 | 3.53 | 17.34 | 4.91 | 3.22 | 3.11 | 2.03 | 11.89 | 6.25 | 2.97 | 4 |
| 85 | 296210 | N74392 | 0.44 | 2.17 | 4.90 | 3.19 | 3.94 | 6.56 | 2.56 | 2.70 | 10.45 | 4 |
| 86 | 40402 | R52089 | 0.02 | 0.09 | 4.82 | 3.44 | 0.53 | 4.63 | 7.12 | 1.57 | 11.65 | 4 |
| 87 | 858153 | AA633811 | 0.05 | 0.24 | 4.82 | 1.99 | 4.80 | 2.74 | 9.28 | 4.42 | 5.71 | 4 |
| 88 | 704697 | AA282253 | 0.27 | 1.26 | 4.71 | 1.92 | 9.73 | 7.83 | 3.55 | 3.98 | 1.23 | 4 |
| 89 | 814306 | AA459318 | 9.65 | 45.42 | 4.71 | 5.23 | 10.10 | 4.41 | 5.28 | 1.16 | 2.07 | 4 |
| 90 | 781233 | AA446316 | 0.02 | 0.10 | 4.70 | 0.48 | 4.83 | 6.38 | 7.51 | 1.30 | 7.69 | 4 |
| 91 | 823794 | AA490263 | 1.70 | 7.94 | 4.66 | 3.60 | 10.81 | 5.89 | 3.38 | 1.85 | 2.40 | 4 |
| 92 | 280170 | N32072 | 12.85 | 59.29 | 4.61 | 5.52 | 5.50 | 9.33 | 1.01 | 1.63 | 4.68 | 4 |
| 93 | 449309 | AA777902 | 2.47 | 11.22 | 4.54 | 4.85 | 3.66 | 3.87 | 9.59 | 2.38 | 2.87 | 4 |
| 94 | 236155 | H61758 | 0.96 | 4.35 | 4.51 | 4.04 | 2.42 | 10.26 | 1.78 | 4.81 | 3.79 | 4 |
| 95 | 296679 | W02256 | 0.94 | 4.15 | 4.41 | 1.85 | 2.47 | 3.73 | 5.40 | 9.60 | 3.44 | 4 |
| 96 | 195387 | R88915 | 0.47 | 2.07 | 4.38 | 5.34 | 6.46 | 6.39 | 1.35 | 5.02 | 1.71 | 4 |
| 97 | 428267 | AA004626 | 1.41 | 6.20 | 4.38 | 2.17 | 5.43 | 7.71 | 1.35 | 3.96 | 5.65 | 4 |
| 98 | 391984 | A1003625 | 4.42 | 19.15 | 4.34 | 3.25 | 3.54 | 4.23 | 10.05 | 2.98 | 1.97 | 4 |
| 99 | 51700 | H22854 | 1.92 | 8.25 | 4.29 | 5.19 | 4.37 | 3.15 | 9.71 | 1.81 | 1.53 | 4 |
| 100 | 754294 | AA479494 | 15.01 | 62.10 | 4.14 | 5.48 | 5.99 | 2.50 | 3.26 | 4.67 | 2.90 | 4 |
| 101 | 210697 | H66840 | 0.75 | 3.08 | 4.13 | 1.97 | 3.35 | 4.59 | 7.43 | 8.18 | 1.26 | 4 |
| 102 | 809456 | AA443090 | 0.02 | 0.10 | 4.10 | 6.87 | 3.20 | 5.81 | 2.50 | 1.75 | 4.46 | 4 |
| 103 | 361659 | W98189 | 6.37 | 26.06 | 4.09 | 2.86 | 6.98 | 3.63 | 4.72 | 4.28 | 2.07 | 4 |
| 104 | 148225 | H13688 | 1.72 | 7.01 | 4.07 | 3.68 | 2.48 | 4.06 | 2.17 | 8.08 | 3.94 | 4 |
| 105 | 417887 | W90128 | 13.56 | 54.72 | 4.04 | 3.89 | 3.21 | 2.71 | 10.52 | 0.69 | 3.20 | 4 |
| 106 | 438531 | AA703046 | 1.44 | 5.81 | 4.03 | 1.27 | 3.01 | 3.96 | 5.13 | 8.53 | 2.31 | 4 |
| 107 | 1031348 | AA609106 | 1.74 | 6.94 | 3.99 | 6.11 | 5.02 | 3.69 | 2.74 | 2.97 | 3.41 | 4 |
| 108 | 1587933 | AA939275 | 0.61 | 2.38 | 3.91 | 2.08 | 5.00 | 4.77 | 3.69 | 6.17 | 1.75 | 4 |
| 109 | 144747 | R76247 | 0.43 | 1.67 | 3.89 | 9.05 | 3.34 | 1.44 | 1.37 | 4.41 | 3.71 | 4 |
| 110 | 321386 | W32272 | 1.23 | 4.75 | 3.84 | 5.19 | 5.74 | 3.81 | 2.10 | 3.38 | 2.84 | 4 |
| 111 | 725285 | AA291742 | 0.05 | 0.19 | 3.78 | 2.57 | 4.47 | 5.19 | 3.29 | 7.01 | 0.18 | 4 |
| 112 | 258404 | N25883 | 0.28 | 1.07 | 3.78 | 6.40 | 3.53 | 3.79 | 6.85 | 1.28 | 0.83 | 4 |
| 113 | 268692 | N25920 | 1.81 | 6.68 | 3.70 | 2.93 | 4.18 | 2.58 | 3.38 | 4.60 | 4.50 | 4 |
| 114 | 283995 | N53378 | 0.93 | 3.42 | 3.66 | 2.34 | 3.33 | 3.82 | 2.44 | 4.10 | 5.95 | 4 |
| 115 | 359781 | AA011320 | 3.58 | 13.06 | 3.65 | 4.67 | 5.61 | 2.55 | 4.74 | 3.85 | 0.49 | 4 |
| 116 | 854701 | AA630104 | 0.98 | 3.56 | 3.65 | 2.92 | 5.15 | 3.36 | 5.17 | 4.30 | 1.00 | 4 |
| 117 | 248886 | H82081 | 0.83 | 3.03 | 3.65 | 4.16 | 2.79 | 3.62 | 5.06 | 3.32 | 2.93 | 4 |
| 118 | 771053 | AA427528 | 10.52 | 38.08 | 3.62 | 3.55 | 5.47 | 3.81 | 5.38 | 2.39 | 1.11 | 4 |
| 119 | 488010 | AA045709 | 0.98 | 3.54 | 3.80 | 1.73 | 6.29 | 3.11 | 1.20 | 3.29 | 5.98 | 4 |
| 120 | 112482 | T90980 | 11.33 | 40.58 | 3.58 | 1.21 | 4.61 | 5.01 | 4.80 | 4.57 | 1.28 | 4 |
| 121 | 86035 | T82842 | 0.03 | 0.10 | 3.58 | 5.81 | 3.02 | 3.15 | 2.19 | 2.78 | 4.53 | 4 |
| 122 | 855632 | AA664104 | 0.96 | 3.38 | 3.52 | 2.09 | 3.77 | 3.88 | 1.92 | 5.82 | 3.66 | 4 |
| 123 | 811914 | AA454986 | 2.96 | 10.39 | 3.51 | 3.92 | 5.10 | 3.87 | 3.79 | 2.79 | 1.62 | 4 |
| 124 | 281371 | N47886 | 0.36 | 1.25 | 3.49 | 5.39 | 3.04 | 3.53 | 2.17 | 2.42 | 4.40 | 4 |
| 125 | 383823 | AA704850 | 9.01 | 31.38 | 3.48 | 2.44 | 3.12 | 4.85 | 1.73 | 3.48 | 5.29 | 4 |
| 126 | 395400 | AA757401 | 2.20 | 7.66 | 3.48 | 2.26 | 5.31 | 3.00 | 4.08 | 4.15 | 2.08 | 4 |
| 127 | 136607 | R63253 | 1.41 | 4.88 | 3.47 | 1.74 | 4.81 | 3.90 | 3.23 | 4.16 | 2.95 | 4 |
| 128 | 289071 | N63809 | 19.65 | 68.00 | 3.46 | 3.37 | 5.09 | 1.36 | 1.59 | 6.09 | 3.28 | 4 |
| 129 | 480203 | AA677481 | 0.55 | 1.89 | 3.45 | 1.61 | 4.23 | 4.13 | 4.28 | 4.93 | 1.55 | 4 |
| 130 | 242061 | H93335 | 2.71 | 9.21 | 3.40 | 5.12 | 6.73 | 3.43 | 3.25 | 0.92 | 0.95 | 4 |
| 131 | 207098 | H48502 | 0.16 | 0.53 | 3.38 | 3.02 | 4.77 | 3.04 | 2.28 | 4.65 | 2.52 | 4 |
| 132 | 396839 | AA758154 | 0.66 | 2.24 | 3.36 | 1.87 | 5.62 | 3.24 | 4.60 | 1.60 | 3.25 | 4 |
| 133 | 209381 | H64095 | 0.97 | 3.25 | 3.35 | 2.04 | 3.28 | 4.07 | 1.86 | 4.90 | 3.98 | 4 |
| 134 | 23897 | R38364 | 0.38 | 1.24 | 3.28 | 1.98 | 3.91 | 5.86 | 3.28 | 1.47 | 3.18 | 4 |
| 135 | 1467905 | AA883504 | 1.18 | 3.84 | 3.27 | 1.87 | 3.82 | 4.55 | 3.37 | 3.67 | 2.04 | 4 |
| 136 | 1556526 | AA935560 | 1.17 | 3.77 | 3.21 | 3.58 | 5.88 | 3.20 | 0.99 | 4.48 | 1.12 | 4 |
| 137 | 491418 | AA115537 | 0.54 | 1.72 | 3.20 | 1.82 | 3.32 | 2.46 | 4.38 | 3.33 | 3.88 | 4 |
| 138 | 744001 | AA629039 | 3.87 | 12.24 | 3.17 | 1.60 | 3.05 | 3.27 | 4.18 | 5.11 | 1.80 | 4 |
| 139 | 293110 | N63848 | 3.38 | 10.70 | 3.17 | 2.38 | 3.94 | 3.44 | 3.86 | 3.55 | 1.84 | 4 |
| 140 | 782047 | AA429602 | 1.17 | 3.67 | 3.14 | 3.26 | 2.51 | 3.73 | 2.89 | 3.22 | 3.21 | 4 |
| 141 | 454689 | AA877206 | 1.02 | 3.16 | 3.08 | 2.09 | 3.15 | 1.69 | 3.87 | 3.10 | 4.59 | 4 |
| 142 | 384293 | AA709370 | 0.50 | 1.53 | 3.05 | 4.26 | 3.84 | 4.05 | 3.20 | 1.06 | 1.87 | 4 |
| 143 | 34091 | R44132 | 0.53 | 1.63 | 3.04 | 2.04 | 3.94 | 3.79 | 3.14 | 3.71 | 1.62 | 4 |
| 144 | 743362 | AA400385 | 0.03 | 0.09 | 3.02 | 3.33 | 3.10 | 2.05 | 4.16 | 0.88 | 4.58 | 4 |
| 145 | 128785 | R10015 | 1.14 | 3.44 | 3.02 | 2.11 | 2.81 | 3.08 | 3.17 | 3.53 | 3.40 | 4 |
| 146 | 429299 | AA007370 | 1.28 | 3.83 | 2.98 | 3.22 | 3.29 | 1.89 | 3.35 | 2.22 | 3.94 | 4 |
| 147 | 136819 | R38465 | 0.47 | 1.37 | 2.94 | 3.15 | 2.95 | 3.25 | 3.12 | 1.85 | 3.33 | 4 |
| 148 | 433350 | AA700604 | 42.83 | 123.16 | 2.88 | 3.66 | 5.22 | 3.12 | 1.22 | 0.47 | 3.55 | 4 |
| 149 | 755276 | AA496349 | 0.11 | 0.30 | 2.74 | 3.57 | 4.62 | 3.03 | 1.81 | 0.36 | 3.03 | 4 |
| 150 | 270917 | N32514 | 0.01 | 0.23 | 25.92 | 3.34 | 1.13 | 1.13 | 14.34 | 134.44 | 1.13 | 3 |
| 151 | 782233 | AA431721 | 0.20 | 3.10 | 15.42 | 64.92 | 1.22 | 0.11 | 0.81 | 3.63 | 22.02 | 3 |
| 152 | 320495 | W16659 | 0.06 | 0.88 | 13.99 | 1.39 | 3.75 | 0.97 | 71.36 | 3.78 | 2.88 | 3 |
| 153 | 293579 | N94111 | 0.96 | 12.90 | 13.42 | 0.73 | 1.52 | 4.77 | 26.47 | 44.95 | 2.05 | 3 |
| 154 | 281039 | N47717 | 0.78 | 9.99 | 12.85 | 43.16 | 25.22 | 5.01 | 2.22 | 0.30 | 1.22 | 3 |
| 155 | 490718 | AA115761 | 0.04 | 0.46 | 12.37 | 46.34 | 6.67 | 1.83 | 0.27 | 18.31 | 0.82 | 3 |
| 156 | 796747 | AA443147 | 0.08 | 0.92 | 11.12 | 0.12 | 0.12 | 2.23 | 55.00 | 5.81 | 3.44 | 3 |
| 157 | 287728 | N59158 | 0.51 | 5.43 | 10.89 | 20.89 | 31.93 | 7.51 | 1.25 | 0.88 | 1.67 | 3 |
| 158 | 282089 | N48259 | 1.90 | 19.34 | 10.17 | 21.89 | 29.40 | 4.51 | 1.88 | 1.07 | 2.28 | 3 |
| 159 | 731290 | AA416627 | 4.08 | 33.97 | 8.32 | 14.84 | 26.99 | 1.81 | 1.28 | 0.78 | 4.21 | 3 |
| 160 | 470216 | AA028987 | 2.95 | 23.62 | 8.02 | 1.97 | 4.75 | 1.05 | 5.39 | 32.26 | 2.68 | 3 |
| 161 | 375827 | AA039851 | 0.07 | 0.57 | 7.75 | 3.54 | 3.42 | 1.63 | 1.38 | 34.08 | 2.42 | 3 |
| 162 | 277714 | N49577 | 0.13 | 0.94 | 7.33 | 0.87 | 3.83 | 1.15 | 32.30 | 5.66 | 0.12 | 3 |
| 163 | 757451 | AA437241 | 0.47 | 3.37 | 7.13 | 4.67 | 30.78 | 1.10 | 3.88 | 0.99 | 1.36 | 3 |
| 164 | 486035 | AA040861 | 5.25 | 35.89 | 8.84 | 2.41 | 4.29 | 2.60 | 21.59 | 8.58 | 1.59 | 3 |
| 165 | 298417 | N74131 | 0.08 | 0.48 | 6.33 | 13.15 | 16.83 | 1.72 | 1.58 | 1.47 | 3.24 | 3 |
| 166 | 666218 | AA233809 | 0.04 | 0.23 | 6.24 | 1.91 | 7.96 | 4.25 | 20.28 | 2.26 | 0.81 | 3 |
| 167 | 757389 | AA437142 | 0.02 | 0.11 | 5.76 | 1.70 | 4.23 | 0.65 | 4.75 | 0.56 | 22.68 | 3 |
| 168 | 196168 | R92362 | 0.59 | 3.32 | 5.60 | 1.71 | 1.65 | 2.16 | 16.65 | 4.09 | 7.34 | 3 |
| 169 | 687667 | AA235370 | 0.81 | 4.56 | 5.59 | 5.88 | 18.59 | 4.47 | 1.32 | 1.05 | 2.26 | 3 |
| 170 | 712950 | AA282273 | 5.93 | 32.98 | 5.56 | 6.81 | 14.24 | 8.62 | 0.72 | 0.88 | 2.12 | 3 |
| 171 | 504761 | AA148735 | 0.08 | 0.43 | 5.45 | 6.26 | 0.51 | 7.72 | 0.13 | 17.97 | 0.10 | 3 |
| 172 | 322443 | W16424 | 0.23 | 1.23 | 5.30 | 3.13 | 4.09 | 0.73 | 20.36 | 0.89 | 2.62 | 3 |
| 173 | 35010 | R45118 | 0.66 | 3.31 | 5.03 | 2.72 | 4.85 | 2.60 | 1.65 | 11.48 | 6.86 | 3 |
| 174 | 782537 | AA448484 | 1.72 | 8.60 | 5.01 | 2.52 | 2.75 | 8.08 | 0.99 | 7.48 | 8.26 | 3 |
| 175 | 277596 | N57002 | 1.48 | 7.34 | 4.97 | 7.68 | 12.12 | 2.17 | 0.90 | 1.13 | 5.84 | 3 |
| 176 | 773208 | AA425650 | 9.71 | 47.09 | 4.85 | 1.82 | 1.90 | 6.84 | 12.13 | 2.89 | 3.52 | 3 |
| 177 | 384865 | AA035745 | 0.44 | 2.09 | 4.78 | 1.52 | 1.24 | 13.33 | 0.96 | 7.22 | 4.39 | 3 |
| 178 | 247587 | N58136 | 0.01 | 0.05 | 4.64 | 1.96 | 14.29 | 1.91 | 4.85 | 3.94 | 0.89 | 3 |
| 179 | 34888 | R19878 | 0.14 | 0.67 | 4.62 | 5.69 | 9.05 | 6.74 | 1.93 | 1.66 | 2.65 | 3 |
| 180 | 72395 | T51539 | 0.08 | 0.36 | 4.59 | 0.67 | 16.34 | 4.47 | 1.37 | 4.55 | 0.13 | 3 |
| 181 | 773204 | AA425692 | 12.74 | 58.08 | 4.56 | 1.47 | 1.49 | 5.37 | 13.00 | 2.45 | 3.58 | 3 |
| 182 | 277327 | N57483 | 0.02 | 0.09 | 4.56 | 1.67 | 6.21 | 2.55 | 8.18 | 1.05 | 7.68 | 3 |
| 183 | 770066 | AA430545 | 7.38 | 33.36 | 4.52 | 7.28 | 8.45 | 8.19 | 0.54 | 0.26 | 2.38 | 3 |
| 184 | 51620 | H19446 | 0.03 | 0.12 | 4.49 | 1.90 | 8.33 | 7.29 | 6.63 | 2.43 | 0.38 | 3 |
| 185 | 266343 | N26556 | 0.70 | 3.10 | 4.44 | 8.62 | 9.86 | 4.53 | 0.84 | 0.94 | 1.83 | 3 |
| 186 | 296170 | W02426 | 0.43 | 1.87 | 4.40 | 4.09 | 12.69 | 6.34 | 0.91 | 1.09 | 1.27 | 3 |
| 187 | 278504 | N66139 | 0.05 | 0.22 | 4.34 | 0.98 | 7.96 | 1.80 | 5.23 | 1.24 | 8.83 | 3 |
| 188 | 526567 | AA128407 | 0.02 | 0.07 | 4.33 | 3.30 | 1.67 | 0.98 | 9.74 | 0.64 | 9.67 | 3 |
| 189 | 809526 | AA454570 | 0.06 | 0.28 | 4.33 | 10.35 | 0.16 | 0.26 | 2.13 | 8.96 | 4.11 | 3 |
| 190 | 292958 | N69499 | 0.05 | 0.22 | 4.31 | 0.46 | 1.96 | 0.68 | 3.72 | 15.21 | 3.80 | 3 |
| 191 | 295997 | N73563 | 0.42 | 1.81 | 4.28 | 7.32 | 9.76 | 3.67 | 2.18 | 1.01 | 1.74 | 3 |
| 192 | 205828 | H57585 | 0.51 | 2.18 | 4.28 | 7.69 | 2.40 | 5.57 | 1.28 | 1.92 | 6.79 | 3 |
| 193 | 67397 | T49325 | 0.01 | 0.06 | 4.16 | 5.29 | 7.99 | 0.68 | 0.90 | 0.68 | 9.45 | 3 |
| 194 | 811028 | AA485373 | 4.96 | 20.42 | 4.12 | 3.45 | 0.81 | 2.68 | 7.56 | 1.37 | 8.82 | 3 |
| 195 | 489637 | AA101878 | 2.86 | 11.62 | 4.06 | 3.07 | 1.98 | 7.47 | 8.01 | 2.44 | 1.39 | 3 |
| 196 | 284824 | N64796 | 1.58 | 6.38 | 4.03 | 2.09 | 6.18 | 6.46 | 2.24 | 5.69 | 1.52 | 3 |
| 197 | 841507 | AA487385 | 1.06 | 4.28 | 4.02 | 10.98 | 2.50 | 4.10 | 3.36 | 1.15 | 2.06 | 3 |
| 198 | 882497 | AA676604 | 0.04 | 0.14 | 3.94 | 0.28 | 7.63 | 0.28 | 9.35 | 0.79 | 5.34 | 3 |
| 199 | 280155 | N47012 | 0.06 | 0.25 | 3.92 | 2.52 | 2.29 | 7.11 | 2.63 | 4.99 | 4.00 | 3 |
| 200 | 1466788 | AA883735 | 0.46 | 1.79 | 3.90 | 2.93 | 3.44 | 5.26 | 7.90 | 1.68 | 2.22 | 3 |
| 201 | 784589 | AA443300 | 0.03 | 0.10 | 3.89 | 3.05 | 1.55 | 3.64 | 1.86 | 1.93 | 11.30 | 3 |
| 202 | 723972 | AA292583 | 0.01 | 0.05 | 3.89 | 3.91 | 7.78 | 1.46 | 8.70 | 0.74 | 0.74 | 3 |
| 203 | 856174 | AA630620 | 0.03 | 0.11 | 3.89 | 6.41 | 6.16 | 0.96 | 0.35 | 1.33 | 8.10 | 3 |
| 204 | 782575 | AA447522 | 0.36 | 1.39 | 3.86 | 8.55 | 7.01 | 2.57 | 0.65 | 0.88 | 3.47 | 3 |
| 205 | 428166 | AA001870 | 2.28 | 8.77 | 3.85 | 1.91 | 3.10 | 4.57 | 10.12 | 1.80 | 1.78 | 3 |
| 206 | 588826 | AA130857 | 1.17 | 4.49 | 3.83 | 2.76 | 2.15 | 2.16 | 8.17 | 4.00 | 3.75 | 3 |
| 207 | 730838 | AA417012 | 0.02 | 0.08 | 3.81 | 7.50 | 0.49 | 0.49 | 7.80 | 2.29 | 4.30 | 3 |
| 208 | 837953 | AA458578 | 7.85 | 29.78 | 3.79 | 3.76 | 5.93 | 1.10 | 2.48 | 6.86 | 2.62 | 3 |
| 209 | 503824 | AA133959 | 0.12 | 0.44 | 3.78 | 1.62 | 0.09 | 1.78 | 6.89 | 5.74 | 6.57 | 3 |
| 210 | 208210 | H65286 | 0.89 | 3.34 | 3.76 | 5.28 | 5.92 | 7.33 | 1.69 | 0.76 | 1.61 | 3 |
| 211 | 427996 | AA001841 | 0.11 | 0.43 | 3.75 | 3.08 | 1.05 | 3.00 | 3.88 | 0.51 | 11.00 | 3 |
| 212 | 123755 | R01304 | 1.84 | 6.90 | 3.75 | 0.50 | 0.29 | 0.34 | 4.24 | 12.61 | 4.50 | 3 |
| 213 | 436153 | AA703249 | 14.14 | 52.21 | 3.69 | 1.02 | 1.89 | 3.14 | 8.23 | 5.06 | 2.80 | 3 |
| 214 | 143062 | R71335 | 0.44 | 1.61 | 3.67 | 2.83 | 6.04 | 2.57 | 2.67 | 3.32 | 4.58 | 3 |
| 215 | 131599 | R23727 | 0.16 | 0.60 | 3.65 | 5.05 | 2.39 | 2.70 | 1.07 | 4.57 | 6.14 | 3 |
| 216 | 240151 | H82706 | 2.33 | 8.49 | 3.64 | 2.83 | 0.64 | 1.92 | 4.91 | 6.33 | 5.22 | 3 |
| 217 | 452116 | AA707219 | 0.44 | 1.61 | 3.63 | 2.41 | 7.49 | 3.46 | 2.29 | 3.62 | 2.52 | 3 |
| 218 | 201125 | R98472 | 1.05 | 3.81 | 3.61 | 3.93 | 4.00 | 2.14 | 6.14 | 2.91 | 2.55 | 3 |
| 219 | 126713 | R07115 | 0.27 | 0.98 | 3.55 | 6.64 | 4.18 | 2.20 | 4.29 | 1.20 | 2.78 | 3 |
| 220 | 768179 | AA424849 | 2.56 | 9.08 | 3.54 | 3.04 | 3.99 | 1.69 | 2.78 | 7.17 | 2.61 | 3 |
| 221 | 250678 | H95976 | 4.09 | 14.42 | 3.52 | 2.95 | 6.36 | 3.23 | 3.77 | 2.33 | 2.51 | 3 |
| 222 | 273499 | N33263 | 0.09 | 0.32 | 3.52 | 3.88 | 2.67 | 2.75 | 6.21 | 4.11 | 1.47 | 3 |
| 223 | 755765 | AA496455 | 1.95 | 6.84 | 3.51 | 2.12 | 3.80 | 3.48 | 7.92 | 1.94 | 1.82 | 3 |
| 224 | 815047 | AA465166 | 2.68 | 9.34 | 3.49 | 2.62 | 1.60 | 1.82 | 4.71 | 3.01 | 7.18 | 3 |
| 225 | 384670 | AA709333 | 4.17 | 14.53 | 3.48 | 3.11 | 2.41 | 3.77 | 7.25 | 2.34 | 2.03 | 3 |
| 226 | 782200 | AA431964 | 1.53 | 5.30 | 3.47 | 2.48 | 6.32 | 2.54 | 3.94 | 2.26 | 3.29 | 3 |
| 227 | 52730 | H29500 | 6.64 | 22.94 | 3.45 | 2.33 | 2.11 | 4.07 | 6.25 | 3.48 | 2.49 | 3 |
| 228 | 125311 | R05810 | 1.92 | 6.62 | 3.44 | 1.07 | 2.84 | 3.69 | 6.68 | 4.33 | 2.05 | 3 |
| 229 | 770880 | AA434404 | 8.05 | 27.68 | 3.44 | 6.26 | 3.15 | 7.87 | 1.10 | 0.49 | 1.77 | 3 |
| 230 | 796268 | AA460827 | 0.55 | 1.89 | 3.43 | 1.43 | 3.68 | 2.58 | 5.27 | 5.89 | 1.71 | 3 |
| 231 | 204146 | H55920 | 2.07 | 7.07 | 3.42 | 2.91 | 3.75 | 7.21 | 4.16 | 1.12 | 1.39 | 3 |
| 232 | 611373 | AA176833 | 0.01 | 0.04 | 3.41 | 9.82 | 3.38 | 1.53 | 3.96 | 0.90 | 0.90 | 3 |
| 233 | 325380 | AA284285 | 0.01 | 0.03 | 3.40 | 1.86 | 1.13 | 5.78 | 5.41 | 1.13 | 5.09 | 3 |
| 234 | 434822 | AA703114 | 0.80 | 2.72 | 3.39 | 5.38 | 1.97 | 4.32 | 1.12 | 1.56 | 6.01 | 3 |
| 235 | 1049033 | AA778675 | 1.87 | 6.35 | 3.39 | 4.98 | 4.75 | 2.95 | 4.55 | 1.19 | 1.94 | 3 |
| 236 | 882483 | AA676598 | 2.75 | 9.30 | 3.38 | 1.34 | 4.86 | 2.99 | 5.61 | 3.78 | 1.72 | 3 |
| 237 | 767130 | AA424535 | 0.49 | 1.67 | 3.38 | 1.02 | 0.74 | 1.14 | 3.10 | 10.17 | 4.12 | 3 |
| 238 | 126321 | R06479 | 0.03 | 0.10 | 3.38 | 2.12 | 4.44 | 3.73 | 7.41 | 0.36 | 2.21 | 3 |
| 239 | 230440 | H75328 | 0.07 | 0.22 | 3.37 | 1.97 | 3.57 | 3.44 | 2.89 | 8.21 | 0.15 | 3 |
| 240 | 624271 | AA181179 | 6.78 | 22.81 | 3.36 | 3.53 | 3.85 | 2.87 | 5.02 | 2.93 | 1.99 | 3 |
| 241 | 123265 | R00284 | 2.30 | 7.72 | 3.35 | 4.22 | 9.13 | 4.42 | 0.68 | 0.96 | 0.68 | 3 |
| 242 | 357236 | W93638 | 0.72 | 2.40 | 3.35 | 5.76 | 2.69 | 1.43 | 1.46 | 3.53 | 5.19 | 3 |
| 243 | 123331 | R00479 | 1.03 | 3.43 | 3.33 | 2.37 | 4.72 | 5.66 | 0.96 | 3.48 | 2.80 | 3 |
| 244 | 240694 | H78134 | 1.85 | 6.17 | 3.33 | 2.79 | 3.07 | 1.59 | 7.73 | 3.58 | 1.21 | 3 |
| 245 | 347182 | W80811 | 0.02 | 0.07 | 3.32 | 0.45 | 5.03 | 5.53 | 4.94 | 2.81 | 1.19 | 3 |
| 246 | 52092 | H23232 | 0.03 | 0.11 | 3.31 | 1.94 | 4.36 | 6.28 | 4.13 | 2.87 | 0.31 | 3 |
| 247 | 160628 | H25019 | 0.41 | 1.36 | 3.30 | 2.02 | 4.53 | 3.17 | 4.45 | 2.72 | 2.93 | 3 |
| 248 | 1505294 | AA905838 | 1.11 | 3.66 | 3.30 | 1.98 | 2.43 | 7.21 | 1.55 | 3.28 | 3.34 | 3 |
| 249 | 262053 | H98812 | 7.69 | 25.26 | 3.28 | 2.38 | 3.42 | 3.49 | 6.75 | 1.98 | 1.68 | 3 |
| 250 | 741067 | AA478436 | 17.59 | 57.76 | 3.28 | 2.99 | 3.39 | 3.14 | 2.34 | 2.25 | 5.58 | 3 |
| 251 | 490178 | AA121266 | 3.50 | 11.46 | 3.28 | 2.64 | 4.34 | 2.28 | 4.85 | 1.83 | 3.72 | 3 |
| 252 | 208741 | H83175 | 0.09 | 0.30 | 3.28 | 2.00 | 4.61 | 3.24 | 2.64 | 2.82 | 4.27 | 3 |
| 253 | 416611 | W88466 | 3.63 | 11.85 | 3.26 | 2.01 | 2.14 | 2.13 | 5.36 | 4.88 | 3.07 | 3 |
| 254 | 416092 | W85881 | 0.50 | 1.62 | 3.26 | 2.00 | 2.31 | 1.93 | 3.05 | 3.85 | 6.62 | 3 |
| 255 | 221561 | H92525 | 0.55 | 1.79 | 3.26 | 1.18 | 1.35 | 3.60 | 1.43 | 8.94 | 5.05 | 3 |
| 256 | 223274 | H86518 | 0.05 | 0.17 | 3.25 | 3.24 | 0.19 | 0.19 | 2.95 | 5.14 | 7.79 | 3 |
| 257 | 44387 | H08525 | 1.17 | 3.78 | 3.24 | 6.67 | 2.69 | 4.15 | 1.74 | 3.08 | 1.09 | 3 |
| 258 | 110787 | T90639 | 0.25 | 0.80 | 3.23 | 2.49 | 3.52 | 2.10 | 3.80 | 2.05 | 5.34 | 3 |
| 259 | 121948 | T97762 | 4.17 | 13.48 | 3.23 | 1.59 | 4.12 | 2.42 | 5.41 | 3.80 | 2.03 | 3 |
| 260 | 782812 | AA448251 | 0.08 | 0.16 | 3.22 | 4.87 | 5.06 | 1.22 | 2.93 | 3.17 | 2.10 | 3 |
| 261 | 145513 | R77718 | 0.67 | 2.16 | 3.22 | 1.90 | 4.01 | 2.67 | 3.29 | 2.99 | 4.45 | 3 |
| 262 | 435319 | AA899931 | 6.14 | 19.70 | 3.21 | 2.30 | 5.27 | 1.85 | 3.83 | 2.74 | 3.25 | 3 |
| 263 | 210698 | H66877 | 1.41 | 4.52 | 3.21 | 1.59 | 2.24 | 3.47 | 5.80 | 5.20 | 0.95 | 3 |
| 264 | 197067 | R93401 | 0.36 | 1.16 | 3.19 | 1.88 | 1.56 | 3.78 | 3.20 | 2.11 | 6.62 | 3 |
| 265 | 416374 | W86202 | 3.18 | 10.13 | 3.19 | 4.89 | 3.63 | 2.43 | 2.00 | 3.26 | 2.91 | 3 |
| 266 | 1422794 | AA827405 | 2.02 | 6.43 | 3.18 | 2.60 | 3.91 | 2.60 | 5.88 | 1.06 | 3.06 | 3 |
| 267 | 137017 | R35665 | 0.03 | 0.08 | 3.18 | 1.47 | 4.27 | 1.59 | 8.08 | 3.23 | 0.45 | 3 |
| 268 | 267834 | N34117 | 0.21 | 0.66 | 3.18 | 2.32 | 3.85 | 2.53 | 3.88 | 3.77 | 2.92 | 3 |
| 269 | 433253 | AA699427 | 5.61 | 17.79 | 3.17 | 4.18 | 4.57 | 2.43 | 1.80 | 1.72 | 4.54 | 3 |
| 270 | 1475734 | AA872692 | 7.39 | 23.26 | 3.15 | 3.45 | 3.26 | 2.34 | 2.32 | 2.73 | 4.79 | 3 |
| 271 | 268876 | N26028 | 0.51 | 1.59 | 3.14 | 1.46 | 6.62 | 3.35 | 1.38 | 3.88 | 2.19 | 3 |
| 272 | 79032 | T61899 | 15.17 | 47.49 | 3.13 | 3.66 | 4.92 | 3.96 | 1.33 | 2.11 | 2.80 | 3 |
| 273 | 771023 | AA427978 | 1.12 | 3.51 | 3.13 | 2.45 | 3.57 | 2.85 | 2.35 | 3.41 | 4.13 | 3 |
| 274 | 811101 | AA485676 | 2.17 | 6.72 | 3.09 | 1.06 | 2.45 | 1.78 | 5.54 | 4.38 | 3.37 | 3 |
| 275 | 270127 | N27935 | 0.66 | 2.04 | 3.09 | 3.94 | 3.31 | 2.15 | 1.27 | 5.53 | 2.34 | 3 |
| 276 | 448117 | AA702480 | 2.65 | 8.16 | 3.08 | 3.50 | 2.77 | 3.84 | 5.73 | 1.38 | 1.27 | 3 |
| 277 | 790874 | AA480557 | 0.48 | 1.46 | 3.07 | 2.06 | 2.12 | 3.31 | 5.09 | 3.43 | 2.39 | 3 |
| 278 | 431298 | AA682831 | 0.48 | 1.47 | 3.05 | 1.65 | 4.32 | 2.32 | 3.80 | 1.77 | 4.45 | 3 |
| 279 | 214824 | H71242 | 1.92 | 5.87 | 3.05 | 2.21 | 3.82 | 1.69 | 1.33 | 5.44 | 3.82 | 3 |
| 280 | 743323 | AA400598 | 0.49 | 1.50 | 3.04 | 4.39 | 4.19 | 1.89 | 1.95 | 2.28 | 3.78 | 3 |
| 281 | 80050 | T83245 | 12.72 | 38.88 | 3.04 | 1.01 | 1.73 | 3.23 | 4.87 | 5.49 | 1.91 | 3 |
| 282 | 1500162 | AA866739 | 1.89 | 5.73 | 3.04 | 2.12 | 4.97 | 1.27 | 4.24 | 1.94 | 3.69 | 3 |
| 283 | 288234 | N29992 | 1.72 | 5.20 | 3.03 | 2.45 | 3.22 | 5.41 | 2.32 | 1.71 | 3.08 | 3 |
| 284 | 199823 | R98561 | 0.51 | 1.54 | 3.03 | 2.99 | 4.10 | 3.93 | 3.02 | 2.70 | 1.44 | 3 |
| 285 | 41305 | R58877 | 19.83 | 60.07 | 3.03 | 2.41 | 3.80 | 2.96 | 4.40 | 3.55 | 1.08 | 3 |
| 286 | 795555 | AA459879 | 1.83 | 4.94 | 3.03 | 3.08 | 5.11 | 2.83 | 4.91 | 1.14 | 1.32 | 3 |
| 287 | 418808 | W88779 | 0.57 | 1.71 | 3.02 | 1.77 | 4.00 | 2.37 | 4.02 | 3.51 | 2.42 | 3 |
| 288 | 242698 | H94262 | 1.16 | 3.50 | 3.00 | 1.70 | 2.11 | 3.21 | 4.15 | 4.88 | 1.97 | 3 |
| 289 | 364352 | AA022498 | 0.03 | 0.08 | 3.00 | 2.52 | 0.38 | 4.22 | 3.89 | 4.83 | 2.18 | 3 |
| 290 | 244050 | N34042 | 0.06 | 0.17 | 3.00 | 1.60 | 3.79 | 1.68 | 2.22 | 3.29 | 5.43 | 3 |
| 291 | 544639 | AA074677 | 1.05 | 3.15 | 2.99 | 1.72 | 3.77 | 3.39 | 3.80 | 2.68 | 2.58 | 3 |
| 292 | 852520 | AA663058 | 17.95 | 53.54 | 2.98 | 3.39 | 3.07 | 2.81 | 4.99 | 2.83 | 0.82 | 3 |
| 293 | 797000 | AA403500 | 0.11 | 0.33 | 2.98 | 1.54 | 3.21 | 1.65 | 5.62 | 1.07 | 4.77 | 3 |
| 294 | 1493252 | AA886870 | 18.18 | 54.08 | 2.96 | 4.82 | 3.25 | 2.81 | 3.26 | 1.52 | 2.20 | 3 |
| 295 | 447416 | AA702335 | 1.15 | 3.41 | 2.96 | 5.58 | 4.11 | 2.12 | 1.04 | 3.10 | 1.84 | 3 |
| 296 | 1475308 | AA877997 | 1.54 | 4.56 | 2.96 | 2.92 | 3.27 | 4.53 | 1.90 | 1.93 | 3.19 | 3 |
| 297 | 194134 | H51039 | 0.22 | 0.86 | 2.95 | 1.35 | 3.39 | 3.86 | 2.74 | 3.89 | 2.47 | 3 |
| 298 | 1458937 | AA883449 | 1.15 | 3.38 | 2.94 | 5.57 | 3.38 | 1.71 | 2.34 | 0.80 | 3.84 | 3 |
| 299 | 201039 | H48278 | 0.83 | 2.42 | 2.93 | 2.76 | 3.98 | 2.31 | 3.06 | 2.04 | 3.43 | 3 |
| 300 | 263769 | H99694 | 0.75 | 2.19 | 2.93 | 3.82 | 7.88 | 3.41 | 1.10 | 1.06 | 0.29 | 3 |
| 301 | 427930 | AA001976 | 0.05 | 0.14 | 2.91 | 0.99 | 3.14 | 6.49 | 0.21 | 4.44 | 2.22 | 3 |
| 302 | 307244 | N95187 | 0.35 | 1.01 | 2.91 | 2.20 | 4.92 | 3.17 | 2.07 | 3.85 | 1.25 | 3 |
| 303 | 810843 | AA458959 | 0.06 | 0.17 | 2.90 | 1.34 | 3.15 | 1.41 | 5.64 | 4.41 | 1.44 | 3 |
| 304 | 1460820 | AA889785 | 3.62 | 10.49 | 2.90 | 2.35 | 2.11 | 3.29 | 1.57 | 3.43 | 4.63 | 3 |
| 305 | 26259 | R20547 | 0.87 | 2.50 | 2.89 | 3.08 | 3.41 | 3.69 | 2.10 | 2.31 | 2.77 | 3 |
| 306 | 366154 | AA062813 | 0.01 | 0.03 | 2.85 | 3.87 | 3.77 | 1.00 | 1.23 | 1.00 | 6.23 | 3 |
| 307 | 282104 | N51498 | 0.93 | 2.66 | 2.84 | 2.66 | 4.75 | 3.79 | 1.52 | 0.39 | 3.94 | 3 |
| 308 | 344308 | W70147 | 0.46 | 1.31 | 2.81 | 3.18 | 3.47 | 5.50 | 1.78 | 1.21 | 1.74 | 3 |
| 309 | 45525 | H08725 | 2.19 | 6.17 | 2.81 | 2.20 | 3.24 | 3.18 | 3.52 | 2.33 | 2.40 | 3 |
| 310 | 196303 | R92435 | 0.04 | 0.11 | 2.80 | 2.56 | 4.53 | 3.04 | 2.46 | 3.30 | 0.95 | 3 |
| 311 | 267495 | N25262 | 1.59 | 4.40 | 2.77 | 3.68 | 3.24 | 2.59 | 3.41 | 1.52 | 2.19 | 3 |
| 312 | 757198 | AA496133 | 0.65 | 1.78 | 2.75 | 3.73 | 4.67 | 3.45 | 1.24 | 1.90 | 1.53 | 3 |
| 313 | 204122 | H61886 | 1.31 | 3.59 | 2.75 | 2.36 | 4.23 | 3.40 | 3.07 | 2.93 | 0.52 | 3 |
| 314 | 839545 | AA489791 | 0.42 | 1.14 | 2.73 | 5.37 | 0.99 | 1.38 | 1.18 | 3.64 | 3.82 | 3 |
| 315 | 809694 | AA454702 | 0.02 | 0.05 | 2.70 | 0.54 | 0.56 | 3.30 | 2.89 | 4.45 | 4.47 | 3 |
| 316 | 126234 | R06362 | 0.30 | 0.81 | 2.69 | 2.13 | 3.70 | 2.31 | 1.13 | 3.58 | 3.32 | 3 |
| 317 | 280407 | N51563 | 0.97 | 2.62 | 2.69 | 1.61 | 3.40 | 1.91 | 3.26 | 3.22 | 2.72 | 3 |
| 318 | 758284 | AA404231 | 0.78 | 2.08 | 2.67 | 1.82 | 2.31 | 1.16 | 3.46 | 3.11 | 4.17 | 3 |
| 319 | 150314 | H00817 | 2.18 | 5.81 | 2.67 | 3.69 | 3.90 | 3.23 | 2.04 | 1.07 | 2.09 | 3 |
| 320 | 288936 | N62696 | 0.04 | 0.11 | 2.67 | 1.59 | 3.47 | 3.00 | 0.99 | 2.99 | 3.97 | 3 |
| 321 | 757440 | AA437226 | 0.05 | 0.13 | 2.67 | 0.21 | 4.19 | 1.79 | 4.52 | 0.21 | 5.08 | 3 |
| 322 | 176554 | H45289 | 0.87 | 2.28 | 2.64 | 1.89 | 2.02 | 3.72 | 3.33 | 3.56 | 1.30 | 3 |
| 323 | 296057 | N69989 | 0.68 | 1.79 | 2.62 | 2.03 | 3.01 | 2.54 | 1.85 | 3.26 | 3.01 | 3 |
| 324 | 755299 | AA496359 | 1.69 | 4.39 | 2.60 | 4.47 | 0.52 | 1.93 | 3.99 | 0.70 | 3.96 | 3 |
| 325 | 725321 | AA291749 | 0.87 | 2.27 | 2.59 | 3.21 | 3.07 | 3.61 | 2.30 | 1.62 | 1.73 | 3 |
| 326 | 1467831 | AA883431 | 1.17 | 3.02 | 2.59 | 1.16 | 1.22 | 4.32 | 0.72 | 3.58 | 4.52 | 3 |
| 327 | 120097 | T95151 | 0.60 | 1.54 | 2.58 | 2.09 | 2.01 | 1.14 | 3.96 | 3.01 | 3.31 | 3 |
| 328 | 159455 | H15746 | 0.02 | 0.04 | 2.58 | 0.62 | 3.70 | 2.31 | 0.64 | 3.72 | 4.51 | 3 |
| 329 | 1460696 | AA868802 | 6.57 | 16.82 | 2.58 | 1.39 | 3.08 | 1.94 | 2.32 | 3.41 | 3.22 | 3 |
| 330 | 757368 | AA437126 | 0.02 | 0.06 | 2.55 | 3.92 | 2.20 | 3.37 | 0.77 | 0.24 | 4.83 | 3 |
| 331 | 450612 | AA882452 | 0.74 | 1.86 | 2.55 | 1.17 | 0.96 | 0.92 | 3.00 | 4.79 | 4.44 | 3 |
| 332 | 342543 | W68141 | 0.02 | 0.04 | 2.53 | 4.17 | 4.44 | 0.58 | 1.17 | 4.50 | 0.30 | 3 |
| 333 | 503741 | AA131466 | 1.05 | 2.65 | 2.53 | 3.31 | 3.85 | 3.58 | 1.37 | 1.19 | 1.85 | 3 |
| 334 | 784130 | AA432075 | 17.73 | 44.77 | 2.52 | 3.17 | 4.60 | 0.76 | 1.56 | 3.28 | 1.79 | 3 |
| 335 | 325370 | W52208 | 0.03 | 0.08 | 2.51 | 3.68 | 3.87 | 2.92 | 1.13 | 0.31 | 3.14 | 3 |
| 336 | 288863 | N82394 | 0.78 | 1.95 | 2.50 | 3.07 | 3.96 | 0.85 | 1.90 | 1.37 | 3.87 | 3 |
| 337 | 344359 | W73377 | 0.07 | 0.18 | 2.46 | 0.90 | 3.52 | 0.14 | 3.08 | 7.02 | 0.08 | 3 |
| 338 | 53391 | R16241 | 1.15 | 2.79 | 2.41 | 3.60 | 3.57 | 3.34 | 1.40 | 1.23 | 1.35 | 3 |
| 339 | 429823 | AA009803 | 0.14 | 0.35 | 2.40 | 3.16 | 3.19 | 1.82 | 1.65 | 1.06 | 3.55 | 3 |
| 340 | 258242 | N30655 | 0.62 | 1.49 | 2.40 | 3.61 | 3.23 | 0.28 | 4.12 | 0.85 | 2.32 | 3 |
| 341 | 255897 | N27366 | 0.09 | 0.20 | 2.31 | 0.60 | 4.21 | 1.00 | 1.52 | 3.40 | 3.14 | 3 |
| 342 | 452068 | AA707125 | 0.88 | 2.03 | 2.30 | 1.29 | 3.24 | 1.70 | 3.19 | 3.01 | 1.39 | 3 |
| 343 | 505779 | AA135870 | 20.69 | 46.64 | 2.25 | 2.43 | 3.80 | 0.16 | 3.57 | 0.38 | 3.18 | 3 |
| 344 | 128508 | R10154 | 0.04 | 0.09 | 2.15 | 1.20 | 3.87 | 0.24 | 3.30 | 0.24 | 4.05 | 3 |
| 345 | 839048 | AA487505 | 0.36 | 0.76 | 2.12 | 4.12 | 3.45 | 0.42 | 3.04 | 0.88 | 0.82 | 3 |
| 346 | 280763 | N50583 | 0.03 | 0.50 | 14.36 | 0.58 | 2.77 | 2.38 | 1.76 | 4.85 | 73.83 | 2 |
| 347 | 890853 | AA158162 | 0.01 | 0.18 | 13.25 | 0.74 | 0.74 | 1.96 | 0.74 | 42.46 | 32.88 | 2 |
| 348 | 951101 | AA620472 | 0.02 | 0.22 | 11.30 | 1.40 | 0.82 | 3.37 | 1.36 | 2.46 | 58.40 | 2 |
| 349 | 377610 | AA055833 | 0.13 | 1.34 | 10.02 | 1.46 | 2.93 | 2.38 | 49.00 | 1.07 | 3.31 | 2 |
| 350 | 50787 | H16833 | 0.06 | 0.50 | 7.90 | 1.60 | 0.16 | 2.38 | 2.67 | 34.78 | 5.82 | 2 |
| 351 | 137984 | R63085 | 0.29 | 2.25 | 7.79 | 8.43 | 33.82 | 0.37 | 0.58 | 1.11 | 2.44 | 2 |
| 352 | 856585 | AA669222 | 0.12 | 0.94 | 7.77 | 1.46 | 0.70 | 1.19 | 9.43 | 32.14 | 1.71 | 2 |
| 353 | 358990 | W92263 | 0.06 | 0.49 | 7.64 | 1.97 | 2.74 | 4.55 | 0.24 | 35.70 | 0.66 | 2 |
| 354 | 430192 | AA010328 | 0.10 | 0.75 | 7.47 | 33.12 | 2.43 | 1.19 | 3.78 | 1.47 | 2.84 | 2 |
| 355 | 151477 | H02837 | 1.19 | 8.16 | 6.87 | 7.32 | 29.35 | 1.05 | 0.49 | 0.46 | 2.55 | 2 |
| 356 | 81331 | T60111 | 0.47 | 3.19 | 6.81 | 15.33 | 19.31 | 2.50 | 1.64 | 0.68 | 1.40 | 2 |
| 357 | 52026 | H22932 | 0.25 | 1.66 | 6.69 | 13.67 | 22.88 | 0.57 | 1.63 | 1.01 | 0.36 | 2 |
| 358 | 859627 | AA666405 | 0.08 | 0.53 | 6.43 | 1.07 | 0.12 | 0.12 | 0.52 | 31.38 | 5.36 | 2 |
| 359 | 429086 | AA005196 | 0.22 | 1.39 | 6.27 | 0.88 | 0.41 | 0.38 | 1.35 | 18.48 | 16.17 | 2 |
| 360 | 264449 | N21233 | 0.06 | 0.32 | 5.48 | 6.98 | 2.35 | 1.09 | 0.55 | 1.30 | 20.60 | 2 |
| 361 | 754358 | AA436142 | 8.25 | 44.96 | 5.45 | 2.04 | 2.77 | 1.77 | 2.77 | 11.30 | 12.03 | 2 |
| 362 | 768226 | AA424900 | 0.06 | 0.31 | 5.18 | 1.16 | 22.31 | 1.49 | 3.68 | 0.91 | 1.50 | 2 |
| 363 | 303139 | N90808 | 0.52 | 2.71 | 5.17 | 4.05 | 23.41 | 0.98 | 1.45 | 0.13 | 0.99 | 2 |
| 364 | 757165 | AA443950 | 0.03 | 0.16 | 5.09 | 0.47 | 1.51 | 0.94 | 20.25 | 2.06 | 5.30 | 2 |
| 365 | 585235 | AA136125 | 18.25 | 89.08 | 4.88 | 7.86 | 14.43 | 2.52 | 1.47 | 0.81 | 2.40 | 2 |
| 366 | 247103 | N53959 | 0.05 | 0.22 | 4.82 | 3.70 | 1.71 | 1.84 | 0.22 | 2.88 | 18.58 | 2 |
| 367 | 234376 | N28268 | 2.94 | 14.16 | 4.81 | 1.46 | 2.58 | 4.82 | 2.82 | 14.34 | 2.88 | 2 |
| 368 | 756405 | AA482119 | 0.04 | 0.21 | 4.73 | 0.22 | 1.45 | 2.24 | 3.15 | 2.40 | 18.90 | 2 |
| 369 | 47355 | H10981 | 0.10 | 0.48 | 4.65 | 14.30 | 0.10 | 0.55 | 0.22 | 9.95 | 2.80 | 2 |
| 370 | 377898 | AA777098 | 3.49 | 15.28 | 4.38 | 5.72 | 2.44 | 11.88 | 1.29 | 2.30 | 2.82 | 2 |
| 371 | 745072 | AA626275 | 0.49 | 1.95 | 4.32 | 6.41 | 14.50 | 1.23 | 1.41 | 1.47 | 0.93 | 2 |
| 372 | 79763 | T63981 | 0.16 | 0.71 | 4.32 | 1.17 | 7.49 | 1.07 | 1.48 | 0.08 | 14.86 | 2 |
| 373 | 429424 | AA007887 | 0.03 | 0.14 | 4.20 | 2.13 | 15.68 | 0.30 | 1.79 | 0.93 | 4.17 | 2 |
| 374 | 265655 | N22838 | 0.09 | 0.39 | 4.17 | 16.96 | 1.24 | 1.97 | 4.20 | 0.56 | 0.11 | 2 |
| 375 | 1606315 | AA991196 | 0.72 | 3.01 | 4.16 | 2.17 | 2.93 | 3.20 | 13.79 | 1.44 | 1.42 | 2 |
| 376 | 1389018 | AA855158 | 0.05 | 0.20 | 4.11 | 11.10 | 10.86 | 0.50 | 0.52 | 0.21 | 1.46 | 2 |
| 377 | 283335 | N51441 | 0.18 | 0.72 | 4.00 | 1.22 | 10.53 | 5.68 | 2.67 | 1.88 | 1.99 | 2 |
| 378 | 284931 | N71861 | 0.02 | 0.07 | 3.99 | 2.68 | 8.36 | 0.83 | 0.57 | 0.92 | 10.57 | 2 |
| 379 | 1031203 | AA809982 | 0.03 | 0.11 | 3.98 | 1.17 | 0.36 | 5.13 | 1.64 | 0.36 | 15.08 | 2 |
| 380 | 758360 | AA404290 | 0.03 | 0.13 | 3.95 | 0.30 | 0.30 | 2.16 | 4.17 | 0.30 | 16.49 | 2 |
| 381 | 178589 | 849053 | 0.05 | 0.20 | 3.96 | 3.16 | 3.00 | 2.59 | 2.97 | 1.40 | 10.58 | 2 |
| 382 | 154477 | R54855 | 1.01 | 3.95 | 3.92 | 2.32 | 1.75 | 3.10 | 1.67 | 2.16 | 12.53 | 2 |
| 383 | 1048998 | AA778636 | 0.14 | 0.57 | 3.91 | 10.73 | 5.96 | 2.98 | 2.22 | 1.03 | 0.56 | 2 |
| 384 | 129589 | R16541 | 0.44 | 1.66 | 3.79 | 2.77 | 13.43 | 1.09 | 0.93 | 3.45 | 1.09 | 2 |
| 385 | 1055636 | AA620927 | 0.19 | 0.72 | 3.75 | 3.32 | 2.92 | 2.59 | 2.72 | 1.71 | 9.27 | 2 |
| 386 | 461699 | AA682274 | 10.27 | 38.51 | 3.75 | 0.81 | 2.56 | 2.98 | 8.45 | 6.06 | 1.64 | 2 |
| 387 | 50692 | H17626 | 0.31 | 1.18 | 3.74 | 1.87 | 2.97 | 2.88 | 2.21 | 5.65 | 6.87 | 2 |
| 388 | 362926 | AA018980 | 1.07 | 4.00 | 3.74 | 2.76 | 5.27 | 1.60 | 11.37 | 0.71 | 0.71 | 2 |
| 389 | 122178 | T98615 | 0.03 | 0.11 | 3.69 | 0.65 | 8.21 | 0.34 | 1.94 | 0.20 | 10.81 | 2 |
| 390 | 248808 | N53214 | 0.56 | 2.06 | 3.69 | 6.45 | 12.29 | 1.20 | 0.84 | 0.52 | 0.85 | 2 |
| 391 | 191866 | 840350 | 0.61 | 2.24 | 3.68 | 2.33 | 6.76 | 2.83 | 2.98 | 5.39 | 1.99 | 2 |
| 392 | 155072 | R71393 | 0.30 | 1.07 | 3.62 | 7.00 | 9.56 | 2.16 | 0.92 | 0.72 | 1.33 | 2 |
| 393 | 190325 | H29897 | 0.70 | 2.51 | 3.59 | 2.78 | 2.71 | 2.31 | 2.92 | 7.51 | 3.33 | 2 |
| 394 | 502436 | AA134882 | 1.10 | 3.94 | 3.58 | 1.28 | 2.12 | 2.17 | 7.62 | 1.52 | 6.75 | 2 |
| 395 | 293689 | N69675 | 0.01 | 0.03 | 3.57 | 6.95 | 1.08 | 1.08 | 1.03 | 10.16 | 1.08 | 2 |
| 396 | 1486752 | AA912071 | 0.70 | 2.48 | 3.57 | 1.94 | 3.07 | 2.18 | 10.40 | 1.10 | 2.70 | 2 |
| 397 | 510136 | AA053239 | 0.03 | 0.10 | 3.52 | 6.17 | 0.37 | 7.88 | 1.98 | 1.85 | 2.87 | 2 |
| 398 | 878284 | AA670280 | 2.85 | 9.83 | 3.45 | 1.42 | 1.16 | 2.71 | 7.18 | 5.86 | 2.39 | 2 |
| 399 | 321470 | W32303 | 1.07 | 3.68 | 3.45 | 3.79 | 2.25 | 7.49 | 2.62 | 2.54 | 1.99 | 2 |
| 400 | 287866 | N82332 | 1.24 | 4.26 | 3.44 | 2.88 | 4.99 | 2.73 | 1.12 | 7.17 | 1.77 | 2 |
| 401 | 258300 | N30680 | 5.10 | 17.12 | 3.36 | 2.43 | 3.55 | 2.76 | 7.38 | 2.52 | 1.53 | 2 |
| 402 | 1048646 | AA608893 | 0.92 | 3.08 | 3.35 | 1.96 | 2.72 | 3.67 | 2.29 | 2.53 | 6.91 | 2 |
| 403 | 289025 | N59826 | 0.55 | 1.82 | 3.35 | 2.20 | 5.29 | 1.81 | 6.92 | 1.99 | 1.88 | 2 |
| 404 | 291706 | N73480 | 0.53 | 1.77 | 3.33 | 4.14 | 1.81 | 8.88 | 2.13 | 1.50 | 1.49 | 2 |
| 405 | 1031568 | AA609304 | 0.08 | 0.25 | 3.30 | 6.38 | 2.97 | 1.77 | 2.61 | 1.62 | 4.45 | 2 |
| 406 | 383676 | AA679118 | 0.13 | 0.41 | 3.28 | 7.55 | 3.06 | 2.77 | 2.09 | 2.39 | 1.81 | 2 |
| 407 | 310493 | W31074 | 6.22 | 20.37 | 3.28 | 2.57 | 2.10 | 2.21 | 4.55 | 5.37 | 2.84 | 2 |
| 408 | 84713 | T74257 | 0.07 | 0.21 | 3.27 | 6.85 | 6.18 | 2.97 | 2.78 | 0.15 | 0.69 | 2 |
| 409 | 219955 | H81817 | 0.19 | 0.61 | 3.24 | 0.75 | 0.51 | 0.73 | 12.99 | 4.14 | 0.35 | 2 |
| 410 | 295741 | W02483 | 0.35 | 1.13 | 3.23 | 2.04 | 2.14 | 1.65 | 2.27 | 4.35 | 6.93 | 2 |
| 411 | 796646 | AA460115 | 9.12 | 29.44 | 3.23 | 1.38 | 0.93 | 1.89 | 8.52 | 1.58 | 5.09 | 2 |
| 412 | 44253 | H05818 | 0.06 | 0.21 | 3.23 | 1.53 | 1.21 | 0.62 | 3.88 | 1.48 | 10.66 | 2 |
| 413 | 139051 | R62926 | 0.44 | 1.43 | 3.23 | 2.91 | 2.95 | 4.57 | 2.09 | 3.85 | 2.98 | 2 |
| 414 | 454543 | AA677023 | 0.83 | 2.67 | 3.22 | 0.89 | 1.39 | 2.00 | 10.50 | 3.02 | 1.53 | 2 |
| 415 | 49315 | H15366 | 0.22 | 0.71 | 3.21 | 0.69 | 1.60 | 1.44 | 5.55 | 7.72 | 2.25 | 2 |
| 416 | 346612 | W74462 | 1.22 | 3.89 | 3.17 | 2.96 | 9.86 | 0.85 | 1.50 | 3.24 | 0.62 | 2 |
| 417 | 263836 | H99766 | 1.11 | 3.52 | 3.17 | 2.49 | 4.47 | 2.58 | 6.53 | 1.43 | 1.53 | 2 |
| 418 | 378793 | AA046883 | 0.72 | 2.28 | 3.15 | 7.40 | 1.58 | 1.99 | 4.43 | 2.52 | 0.96 | 2 |
| 419 | 283617 | N52875 | 1.18 | 3.69 | 3.13 | 0.71 | 1.64 | 1.65 | 7.85 | 4.94 | 2.01 | 2 |
| 420 | 1684274 | A1003028 | 1.45 | 4.54 | 3.13 | 5.71 | 7.10 | 2.75 | 1.53 | 0.65 | 1.06 | 2 |
| 421 | 784212 | AA446864 | 1.91 | 5.95 | 3.11 | 2.46 | 2.18 | 2.50 | 3.58 | 6.73 | 1.25 | 2 |
| 422 | 41214 | R56898 | 0.03 | 0.11 | 3.11 | 0.98 | 3.88 | 1.59 | 2.55 | 0.17 | 9.50 | 2 |
| 423 | 361688 | W96197 | 0.03 | 0.08 | 3.10 | 7.63 | 1.12 | 4.50 | 2.52 | 0.37 | 2.46 | 2 |
| 424 | 149013 | R82299 | 7.60 | 23.51 | 3.09 | 0.77 | 1.07 | 3.65 | 8.67 | 2.39 | 2.00 | 2 |
| 425 | 392399 | AA707935 | 0.52 | 1.59 | 3.09 | 2.36 | 2.94 | 2.93 | 2.19 | 4.07 | 4.03 | 2 |
| 426 | 347615 | W81526 | 0.07 | 0.20 | 3.08 | 12.89 | 0.30 | 0.86 | 3.29 | 0.17 | 0.99 | 2 |
| 427 | 1030779 | AA609004 | 0.52 | 1.61 | 3.08 | 1.29 | 1.48 | 1.52 | 1.66 | 4.06 | 8.48 | 2 |
| 428 | 364555 | AA022601 | 1.84 | 5.67 | 3.07 | 5.36 | 2.73 | 1.58 | 1.33 | 5.86 | 1.60 | 2 |
| 429 | 743688 | AA629324 | 1.27 | 3.90 | 3.07 | 2.60 | 2.21 | 5.26 | 5.17 | 1.89 | 1.28 | 2 |
| 430 | 141726 | R69584 | 0.04 | 0.13 | 3.07 | 8.46 | 2.87 | 3.49 | 1.95 | 1.40 | 0.24 | 2 |
| 431 | 796147 | AA460986 | 2.96 | 9.08 | 3.07 | 2.49 | 2.65 | 2.36 | 1.77 | 4.31 | 4.82 | 2 |
| 432 | 813501 | AA456078 | 0.60 | 1.85 | 3.07 | 1.76 | 2.60 | 4.93 | 1.72 | 2.04 | 5.35 | 2 |
| 433 | 138047 | R53757 | 0.35 | 1.07 | 3.04 | 2.90 | 1.81 | 2.18 | 2.33 | 4.18 | 4.86 | 2 |
| 434 | 22600 | T87235 | 0.12 | 0.37 | 3.04 | 3.96 | 5.88 | 1.86 | 2.92 | 2.46 | 1.15 | 2 |
| 435 | 201818 | H48318 | 0.16 | 0.48 | 3.04 | 2.44 | 5.47 | 4.57 | 1.88 | 2.74 | 1.14 | 2 |
| 436 | 345781 | W72671 | 0.92 | 2.80 | 3.04 | 2.94 | 5.24 | 1.48 | 2.88 | 3.18 | 2.50 | 2 |
| 437 | 42747 | R61847 | 0.22 | 0.67 | 3.03 | 1.17 | 1.54 | 1.10 | 9.90 | 3.38 | 1.07 | 2 |
| 438 | 743230 | AA400234 | 8.50 | 25.66 | 3.02 | 2.49 | 2.16 | 1.72 | 2.64 | 4.52 | 4.58 | 2 |
| 439 | 281580 | H98694 | 8.42 | 25.35 | 3.01 | 2.74 | 2.82 | 1.36 | 2.36 | 4.05 | 4.73 | 2 |
| 440 | 357628 | W94121 | 0.12 | 0.37 | 3.01 | 3.08 | 1.37 | 8.10 | 2.01 | 2.27 | 1.23 | 2 |
| 441 | 701766 | AA292655 | 4.22 | 12.71 | 3.01 | 1.74 | 5.33 | 1.83 | 2.67 | 2.83 | 3.85 | 2 |
| 442 | 195487 | R92163 | 1.07 | 3.20 | 3.00 | 2.24 | 4.24 | 4.46 | 1.57 | 2.77 | 2.74 | 2 |
| 443 | 460164 | AA676920 | 0.44 | 1.32 | 2.99 | 1.42 | 2.73 | 1.25 | 1.76 | 5.99 | 4.80 | 2 |
| 444 | 788239 | AA478452 | 1.47 | 4.37 | 2.98 | 4.17 | 6.87 | 1.66 | 1.67 | 2.47 | 1.06 | 2 |
| 445 | 385813 | AA025601 | 0.06 | 0.18 | 2.97 | 2.12 | 6.90 | 1.16 | 4.61 | 0.14 | 2.91 | 2 |
| 446 | 346889 | W79834 | 7.57 | 22.52 | 2.97 | 2.25 | 2.76 | 1.65 | 2.96 | 4.30 | 3.91 | 2 |
| 447 | 143790 | R76782 | 2.09 | 6.21 | 2.97 | 2.52 | 2.84 | 2.73 | 4.48 | 3.12 | 2.14 | 2 |
| 448 | 50530 | H17919 | 0.35 | 1.04 | 2.96 | 1.02 | 2.38 | 1.71 | 3.99 | 6.14 | 2.52 | 2 |
| 449 | 208082 | H62583 | 4.22 | 12.48 | 2.96 | 2.26 | 6.36 | 2.48 | 4.67 | 0.81 | 1.18 | 2 |
| 450 | 490525 | AA126468 | 0.07 | 0.20 | 2.95 | 0.16 | 1.59 | 10.45 | 1.71 | 0.15 | 3.62 | 2 |
| 451 | 264157 | N20602 | 0.09 | 0.26 | 2.95 | 4.52 | 5.80 | 2.94 | 2.25 | 0.12 | 2.05 | 2 |
| 452 | 133440 | R27450 | 1.72 | 5.05 | 2.94 | 2.28 | 2.49 | 1.50 | 5.90 | 3.21 | 2.23 | 2 |
| 453 | 416889 | W88832 | 0.52 | 1.52 | 2.92 | 1.91 | 5.15 | 4.77 | 1.53 | 1.35 | 2.83 | 2 |
| 454 | 271076 | N29918 | 2.08 | 6.09 | 2.92 | 4.05 | 4.90 | 2.55 | 2.04 | 1.62 | 2.38 | 2 |
| 455 | 452320 | AA704777 | 1.07 | 3.13 | 2.92 | 2.22 | 2.90 | 2.16 | 3.07 | 2.67 | 4.48 | 2 |
| 456 | 814798 | AA455235 | 25.12 | 73.30 | 2.92 | 3.83 | 7.12 | 1.66 | 2.07 | 1.28 | 1.58 | 2 |
| 457 | 417908 | W90520 | 5.97 | 17.42 | 2.92 | 2.62 | 8.06 | 2.91 | 4.81 | 0.62 | 0.47 | 2 |
| 458 | 745283 | AA825567 | 5.50 | 16.00 | 2.91 | 2.17 | 1.35 | 1.50 | 2.45 | 5.86 | 4.13 | 2 |
| 459 | 50772 | H16803 | 10.55 | 30.44 | 2.89 | 3.09 | 5.75 | 2.12 | 2.83 | 1.37 | 2.15 | 2 |
| 460 | 51606 | H18938 | 0.68 | 1.96 | 2.88 | 2.55 | 3.73 | 3.99 | 2.18 | 2.15 | 2.70 | 2 |
| 461 | 204684 | H57135 | 0.83 | 2.38 | 2.88 | 1.27 | 2.47 | 2.59 | 4.88 | 4.06 | 2.03 | 2 |
| 462 | 731002 | AA418884 | 1.11 | 3.20 | 2.87 | 2.09 | 3.10 | 4.25 | 2.58 | 2.78 | 2.46 | 2 |
| 463 | 124891 | R06119 | 0.85 | 2.43 | 2.87 | 2.68 | 3.84 | 3.59 | 2.24 | 2.73 | 2.16 | 2 |
| 464 | 428138 | AA002007 | 0.55 | 1.58 | 2.87 | 2.46 | 3.04 | 2.60 | 5.16 | 2.82 | 1.11 | 2 |
| 465 | 45587 | H07934 | 1.69 | 4.82 | 2.85 | 2.11 | 5.17 | 3.34 | 2.83 | 1.61 | 2.04 | 2 |
| 466 | 731254 | AA420989 | 0.05 | 0.13 | 2.84 | 2.74 | 1.81 | 3.99 | 1.81 | 2.21 | 4.51 | 2 |
| 467 | 48055 | H08850 | 0.74 | 2.09 | 2.84 | 0.41 | 0.85 | 0.73 | 4.40 | 10.07 | 0.75 | 2 |
| 468 | 626385 | AA189113 | 29.81 | 84.34 | 2.83 | 2.15 | 2.80 | 3.18 | 8.89 | 1.20 | 0.76 | 2 |
| 469 | 276450 | N34824 | 0.47 | 1.32 | 2.82 | 8.04 | 3.19 | 1.65 | 1.41 | 2.46 | 2.16 | 2 |
| 470 | 480435 | AA677581 | 3.97 | 11.17 | 2.82 | 2.79 | 3.27 | 2.11 | 2.34 | 4.55 | 1.85 | 2 |
| 471 | 41825 | R54109 | 0.07 | 0.21 | 2.82 | 0.83 | 4.13 | 2.29 | 2.36 | 0.14 | 7.15 | 2 |
| 472 | 682058 | AA256458 | 1.88 | 5.21 | 2.80 | 4.23 | 4.71 | 2.40 | 2.75 | 0.67 | 2.03 | 2 |
| 473 | 754408 | AA436187 | 0.03 | 0.09 | 2.78 | 5.62 | 0.31 | 1.97 | 2.16 | 4.95 | 1.67 | 2 |
| 474 | 49944 | H29215 | 13.79 | 38.31 | 2.78 | 2.12 | 2.47 | 2.22 | 3.18 | 4.00 | 2.68 | 2 |
| 475 | 291222 | N72198 | 0.08 | 0.23 | 2.77 | 1.48 | 3.14 | 4.59 | 2.82 | 1.71 | 2.89 | 2 |
| 476 | 769857 | AA430387 | 1.25 | 3.45 | 2.76 | 3.20 | 2.91 | 2.93 | 0.95 | 2.38 | 4.21 | 2 |
| 477 | 195875 | R92216 | 0.59 | 1.64 | 2.76 | 0.99 | 2.80 | 4.66 | 2.49 | 2.42 | 3.19 | 2 |
| 478 | 731048 | AA421275 | 0.02 | 0.05 | 2.76 | 0.58 | 2.17 | 0.86 | 4.74 | 2.36 | 5.84 | 2 |
| 479 | 812965 | AA464600 | 0.87 | 2.41 | 2.76 | 4.36 | 1.66 | 2.84 | 3.65 | 1.52 | 2.70 | 2 |
| 480 | 809789 | AA454756 | 0.17 | 0.46 | 2.76 | 1.66 | 5.93 | 2.60 | 3.44 | 1.72 | 1.19 | 2 |
| 481 | 289513 | N62745 | 0.02 | 0.05 | 2.75 | 0.51 | 3.74 | 2.54 | 0.51 | 2.26 | 6.97 | 2 |
| 482 | 327239 | AA284307 | 0.50 | 1.37 | 2.75 | 2.08 | 1.72 | 3.12 | 1.74 | 1.08 | 6.80 | 2 |
| 483 | 23588 | R38369 | 1.07 | 2.96 | 2.75 | 2.22 | 4.10 | 3.71 | 2.22 | 1.48 | 2.78 | 2 |
| 484 | 153617 | R48833 | 1.27 | 3.49 | 2.75 | 1.72 | 3.42 | 2.33 | 2.85 | 1.79 | 4.41 | 2 |
| 485 | 491435 | AA150435 | 0.28 | 0.78 | 2.75 | 0.83 | 4.33 | 2.24 | 4.29 | 1.74 | 2.98 | 2 |
| 486 | 236282 | H61193 | 0.12 | 0.34 | 2.75 | 1.13 | 0.08 | 0.98 | 0.55 | 8.93 | 4.84 | 2 |
| 487 | 781342 | AA448390 | 0.08 | 0.22 | 2.75 | 2.79 | 0.80 | 1.56 | 3.17 | 0.83 | 7.34 | 2 |
| 488 | 786525 | AA452113 | 0.81 | 2.21 | 2.74 | 7.49 | 3.35 | 0.82 | 1.98 | 1.26 | 1.51 | 2 |
| 489 | 742082 | AA405769 | 0.13 | 0.35 | 2.74 | 2.57 | 4.19 | 4.36 | 2.31 | 1.07 | 1.93 | 2 |
| 490 | 39821 | R53428 | 12.95 | 35.29 | 2.73 | 2.05 | 2.54 | 2.17 | 3.08 | 4.23 | 2.31 | 2 |
| 491 | 40338 | R53072 | 2.39 | 6.50 | 2.73 | 2.54 | 4.72 | 4.92 | 2.58 | 0.83 | 0.77 | 2 |
| 492 | 194638 | R84398 | 10.30 | 28.05 | 2.72 | 2.21 | 1.67 | 1.64 | 1.82 | 4.30 | 4.69 | 2 |
| 493 | 149406 | H01516 | 3.58 | 9.74 | 2.72 | 1.80 | 2.57 | 3.46 | 4.97 | 1.74 | 1.77 | 2 |
| 494 | 74223 | T48412 | 6.45 | 17.51 | 2.71 | 3.86 | 6.49 | 1.22 | 0.80 | 1.93 | 1.99 | 2 |
| 495 | 454798 | AA677295 | 0.71 | 1.94 | 2.71 | 2.51 | 1.34 | 3.04 | 1.20 | 0.70 | 7.49 | 2 |
| 496 | 428338 | AA005153 | 1.30 | 3.51 | 2.70 | 1.67 | 3.06 | 2.89 | 2.48 | 3.61 | 2.51 | 2 |
| 497 | 853505 | AA663556 | 0.52 | 1.40 | 2.70 | 1.84 | 2.59 | 3.68 | 1.98 | 2.26 | 3.86 | 2 |
| 498 | 281204 | H98218 | 0.05 | 0.14 | 2.70 | 4.16 | 4.07 | 1.82 | 2.64 | 1.19 | 2.32 | 2 |
| 499 | 1638971 | A1017332 | 1.32 | 3.57 | 2.69 | 1.60 | 2.33 | 3.00 | 5.58 | 1.99 | 1.66 | 2 |
| 500 | 292399 | N68399 | 2.00 | 5.39 | 2.69 | 3.78 | 4.41 | 1.27 | 2.53 | 1.43 | 2.71 | 2 |
| 501 | 878281 | AA670286 | 4.02 | 10.81 | 2.69 | 2.74 | 3.62 | 1.27 | 1.97 | 4.20 | 2.33 | 2 |
| 502 | 194364 | H50677 | 14.95 | 40.11 | 2.68 | 1.36 | 3.04 | 2.45 | 1.92 | 4.73 | 2.60 | 2 |
| 503 | 625924 | AA188895 | 0.08 | 0.21 | 2.68 | 0.63 | 2.98 | 7.53 | 3.18 | 0.92 | 0.82 | 2 |
| 504 | 810328 | AA464143 | 0.28 | 0.75 | 2.67 | 3.26 | 2.72 | 2.12 | 2.74 | 2.16 | 3.04 | 2 |
| 505 | 50114 | H16743 | 2.07 | 5.53 | 2.67 | 2.59 | 6.02 | 0.45 | 5.94 | 0.35 | 0.70 | 2 |
| 506 | 743731 | AA629357 | 0.28 | 0.76 | 2.67 | 2.85 | 1.51 | 4.67 | 0.86 | 4.27 | 1.88 | 2 |
| 507 | 452047 | AA707118 | 0.41 | 1.08 | 2.67 | 1.41 | 6.37 | 1.25 | 3.28 | 1.41 | 2.29 | 2 |
| 508 | 361587 | AA016980 | 0.88 | 2.34 | 2.65 | 2.25 | 2.02 | 1.71 | 1.76 | 3.99 | 4.19 | 2 |
| 509 | 283142 | N51335 | 4.33 | 11.49 | 2.65 | 1.56 | 2.50 | 0.65 | 5.66 | 3.31 | 2.24 | 2 |
| 510 | 342721 | W88291 | 0.58 | 1.53 | 2.65 | 1.63 | 2.24 | 3.17 | 6.41 | 1.43 | 1.03 | 2 |
| 511 | 530185 | AA111989 | 0.31 | 0.83 | 2.65 | 4.40 | 3.73 | 2.69 | 2.49 | 0.20 | 2.38 | 2 |
| 512 | 395809 | AA757588 | 0.03 | 0.07 | 2.65 | 7.24 | 3.66 | 2.09 | 1.38 | 0.84 | 0.68 | 2 |
| 513 | 898318 | AA568828 | 2.46 | 6.52 | 2.65 | 3.03 | 4.78 | 1.15 | 2.49 | 1.44 | 3.00 | 2 |
| 514 | 241787 | H91721 | 0.16 | 0.42 | 2.65 | 1.72 | 3.98 | 3.91 | 1.86 | 1.88 | 2.52 | 2 |
| 515 | 1638108 | AI015679 | 2.30 | 6.08 | 2.65 | 2.96 | 2.48 | 3.78 | 1.64 | 3.28 | 1.73 | 2 |
| 516 | 201172 | R98487 | 0.67 | 1.76 | 2.65 | 1.56 | 4.16 | 2.12 | 1.90 | 2.76 | 3.37 | 2 |
| 517 | 229290 | H63865 | 0.70 | 1.86 | 2.64 | 1.33 | 2.40 | 1.84 | 1.57 | 4.53 | 4.38 | 2 |
| 518 | 342164 | W63778 | 0.04 | 0.10 | 2.64 | 0.60 | 0.26 | 5.08 | 1.19 | 0.26 | 8.46 | 2 |
| 519 | 49394 | H15855 | 0.51 | 1.35 | 2.64 | 2.46 | 4.41 | 2.08 | 2.86 | 3.04 | 0.98 | 2 |
| 520 | 85320 | T71889 | 6.68 | 17.60 | 2.64 | 1.58 | 1.93 | 4.01 | 2.90 | 3.08 | 2.30 | 2 |
| 521 | 115143 | T86708 | 5.36 | 14.12 | 2.63 | 3.65 | 6.57 | 1.85 | 1.65 | 0.94 | 1.14 | 2 |
| 522 | 31969 | R41994 | 0.74 | 1.96 | 2.63 | 1.11 | 1.08 | 1.83 | 1.88 | 5.62 | 4.26 | 2 |
| 523 | 362402 | AA018449 | 9.88 | 25.98 | 2.63 | 2.80 | 4.67 | 2.27 | 3.51 | 1.03 | 1.50 | 2 |
| 524 | 283049 | N20046 | 0.35 | 0.91 | 2.63 | 1.09 | 4.25 | 1.56 | 7.15 | 0.95 | 0.77 | 2 |
| 525 | 31299 | R42946 | 0.73 | 1.92 | 2.63 | 1.79 | 3.06 | 2.41 | 3.54 | 2.21 | 2.76 | 2 |
| 526 | 62112 | T41077 | 3.92 | 10.27 | 2.62 | 3.22 | 2.98 | 1.49 | 3.17 | 2.47 | 2.40 | 2 |
| 527 | 49728 | H29198 | 0.70 | 1.83 | 2.61 | 2.66 | 2.18 | 3.18 | 1.50 | 2.17 | 3.98 | 2 |
| 528 | 295723 | N66925 | 2.82 | 7.37 | 2.61 | 2.25 | 2.74 | 3.72 | 1.30 | 2.40 | 3.25 | 2 |
| 529 | 447715 | AA702781 | 14.05 | 36.63 | 2.61 | 2.78 | 3.25 | 1.52 | 4.68 | 2.16 | 1.26 | 2 |
| 530 | 898070 | AA598779 | 0.92 | 2.40 | 2.61 | 1.58 | 2.75 | 1.77 | 2.53 | 3.64 | 3.36 | 2 |
| 531 | 60540 | T40531 | 1.12 | 2.91 | 2.60 | 2.02 | 2.15 | 2.44 | 2.01 | 3.34 | 3.66 | 2 |
| 532 | 288999 | N62726 | 3.95 | 10.29 | 2.60 | 2.27 | 3.93 | 1.45 | 3.80 | 2.10 | 2.06 | 2 |
| 533 | 290346 | N82293 | 1.53 | 3.99 | 2.60 | 1.93 | 3.19 | 1.53 | 4.80 | 1.96 | 2.40 | 2 |
| 534 | 51865 | H23187 | 0.05 | 0.12 | 2.59 | 2.52 | 1.93 | 1.70 | 3.83 | 3.45 | 2.14 | 2 |
| 535 | 815124 | AA481491 | 1.93 | 5.02 | 2.59 | 1.28 | 1.68 | 2.69 | 3.39 | 4.11 | 2.42 | 2 |
| 536 | 428296 | AA004944 | 0.60 | 1.58 | 2.59 | 1.85 | 2.54 | 2.85 | 1.59 | 3.23 | 3.47 | 2 |
| 537 | 51607 | H19427 | 0.15 | 0.40 | 2.58 | 2.50 | 5.88 | 3.09 | 0.82 | 2.24 | 0.98 | 2 |
| 538 | 283461 | N50647 | 0.06 | 0.14 | 2.58 | 1.74 | 2.31 | 0.18 | 0.37 | 7.88 | 3.22 | 2 |
| 539 | 283190 | N51357 | 0.07 | 0.19 | 2.58 | 1.89 | 5.87 | 2.69 | 3.96 | 0.14 | 0.92 | 2 |
| 540 | 74511 | T59024 | 10.71 | 27.60 | 2.58 | 3.63 | 2.79 | 2.02 | 4.23 | 1.30 | 1.49 | 2 |
| 541 | 300051 | N78927 | 1.59 | 4.10 | 2.57 | 3.34 | 0.28 | 0.12 | 0.88 | 10.78 | 0.24 | 2 |
| 542 | 482354 | AA705525 | 1.39 | 3.59 | 2.57 | 0.98 | 1.51 | 1.84 | 3.37 | 5.79 | 1.95 | 2 |
| 543 | 243159 | H94471 | 1.79 | 4.60 | 2.57 | 1.22 | 1.91 | 1.52 | 4.66 | 4.97 | 1.15 | 2 |
| 544 | 824896 | AA488893 | 0.07 | 0.19 | 2.57 | 4.48 | 2.47 | 2.87 | 3.13 | 1.36 | 1.13 | 2 |
| 545 | 884283 | AA669750 | 9.33 | 23.95 | 2.57 | 1.33 | 1.70 | 3.19 | 1.45 | 5.51 | 2.23 | 2 |
| 546 | 260707 | H97186 | 0.37 | 0.96 | 2.56 | 1.71 | 3.53 | 2.59 | 2.31 | 3.57 | 1.65 | 2 |
| 547 | 140197 | R66101 | 0.29 | 0.74 | 2.56 | 2.80 | 5.19 | 2.09 | 0.54 | 3.25 | 1.49 | 2 |
| 548 | 344618 | W74725 | 0.07 | 0.18 | 2.56 | 4.68 | 2.29 | 2.79 | 3.32 | 1.11 | 1.17 | 2 |
| 549 | 270535 | N33228 | 0.49 | 1.25 | 2.55 | 1.19 | 1.84 | 3.08 | 1.95 | 4.73 | 2.53 | 2 |
| 550 | 811023 | AA485380 | 0.22 | 0.56 | 2.55 | 1.72 | 1.10 | 1.28 | 1.41 | 5.94 | 3.87 | 2 |
| 551 | 208413 | H62162 | 0.34 | 0.86 | 2.55 | 3.23 | 2.89 | 0.80 | 1.04 | 2.81 | 4.51 | 2 |
| 552 | 290227 | N82271 | 3.70 | 9.39 | 2.54 | 2.58 | 3.35 | 0.81 | 2.49 | 3.30 | 2.72 | 2 |
| 553 | 305677 | N89973 | 1.10 | 2.79 | 2.54 | 0.77 | 2.86 | 1.99 | 2.48 | 3.54 | 3.60 | 2 |
| 554 | 298554 | N70592 | 2.03 | 5.15 | 2.54 | 2.50 | 3.69 | 1.78 | 4.20 | 1.46 | 1.59 | 2 |
| 555 | 79000 | T61938 | 6.02 | 15.26 | 2.54 | 3.54 | 2.61 | 3.14 | 1.88 | 1.68 | 2.38 | 2 |
| 556 | 321455 | W45014 | 0.57 | 1.44 | 2.54 | 1.44 | 4.26 | 1.05 | 1.58 | 5.24 | 1.68 | 2 |
| 557 | 1456825 | AA864704 | 3.91 | 9.90 | 2.53 | 1.95 | 1.48 | 2.26 | 3.11 | 2.52 | 3.89 | 2 |
| 558 | 796723 | AA443140 | 0.14 | 0.34 | 2.53 | 2.23 | 3.90 | 1.93 | 1.64 | 4.54 | 0.95 | 2 |
| 559 | 85670 | T62072 | 0.80 | 2.01 | 2.53 | 3.12 | 3.59 | 1.80 | 2.56 | 1.73 | 2.36 | 2 |
| 560 | 365738 | AA025538 | 1.74 | 4.38 | 2.52 | 2.94 | 3.50 | 1.27 | 3.56 | 1.90 | 1.94 | 2 |
| 561 | 489722 | AA099593 | 3.24 | 8.15 | 2.51 | 2.69 | 1.78 | 3.26 | 1.83 | 3.20 | 2.32 | 2 |
| 562 | 1468074 | AA889397 | 0.50 | 1.26 | 2.51 | 1.77 | 1.90 | 2.87 | 3.11 | 3.36 | 2.05 | 2 |
| 563 | 262663 | H88329 | 1.39 | 3.50 | 2.51 | 3.86 | 4.71 | 0.94 | 2.23 | 1.76 | 1.57 | 2 |
| 564 | 36393 | R25823 | 11.96 | 29.92 | 2.50 | 3.52 | 8.36 | 1.90 | 1.39 | 0.86 | 1.01 | 2 |
| 565 | 460039 | AA676740 | 2.76 | 6.89 | 2.50 | 1.35 | 1.80 | 1.09 | 4.19 | 4.77 | 1.79 | 2 |
| 566 | 590148 | AA156030 | 0.11 | 0.27 | 2.50 | 2.80 | 0.09 | 4.82 | 2.90 | 3.11 | 1.27 | 2 |
| 567 | 272600 | N35922 | 1.96 | 4.89 | 2.50 | 2.26 | 3.56 | 3.63 | 1.29 | 2.62 | 1.61 | 2 |
| 568 | 345559 | W73892 | 1.10 | 2.74 | 2.50 | 1.43 | 1.30 | 2.43 | 4.87 | 3.26 | 1.69 | 2 |
| 569 | 1461228 | AA683299 | 0.94 | 2.35 | 2.49 | 1.18 | 1.68 | 2.43 | 3.23 | 3.94 | 2.48 | 2 |
| 570 | 487723 | AA043552 | 2.47 | 6.13 | 2.48 | 1.32 | 2.39 | 3.08 | 3.67 | 2.69 | 1.75 | 2 |
| 571 | 436535 | AA703048 | 0.74 | 1.83 | 2.48 | 1.78 | 1.10 | 1.39 | 4.47 | 3.20 | 2.96 | 2 |
| 572 | 823694 | AA489639 | 0.69 | 1.71 | 2.48 | 3.17 | 2.83 | 2.41 | 3.29 | 1.85 | 1.34 | 2 |
| 573 | 130758 | R22088 | 0.48 | 1.18 | 2.48 | 1.01 | 2.44 | 2.81 | 4.55 | 3.01 | 1.05 | 2 |
| 574 | 50635 | H17921 | 0.38 | 0.93 | 2.47 | 2.13 | 4.23 | 3.13 | 2.67 | 1.93 | 0.76 | 2 |
| 575 | 278483 | N66132 | 9.84 | 24.32 | 2.47 | 2.30 | 3.42 | 2.22 | 3.78 | 1.28 | 1.84 | 2 |
| 576 | 209194 | H63952 | 0.86 | 1.70 | 2.47 | 3.23 | 5.03 | 0.73 | 2.49 | 2.35 | 1.00 | 2 |
| 577 | 308466 | N95495 | 7.94 | 19.83 | 2.47 | 2.01 | 3.49 | 2.05 | 4.93 | 1.49 | 0.85 | 2 |
| 578 | 785849 | AA449120 | 0.03 | 0.07 | 2.46 | 3.20 | 5.99 | 1.70 | 2.59 | 0.95 | 0.35 | 2 |
| 579 | 201931 | H52446 | 0.15 | 0.37 | 2.46 | 1.18 | 1.24 | 4.12 | 5.65 | 0.95 | 1.63 | 2 |
| 580 | 289570 | N62766 | 6.23 | 15.34 | 2.46 | 1.31 | 3.10 | 3.00 | 3.31 | 2.60 | 1.46 | 2 |
| 581 | 67069 | T70438 | 0.62 | 1.53 | 2.46 | 7.96 | 3.28 | 1.04 | 0.96 | 0.82 | 0.71 | 2 |
| 582 | 25061 | R38944 | 14.56 | 35.84 | 2.46 | 1.10 | 3.69 | 3.77 | 1.95 | 2.40 | 1.86 | 2 |
| 583 | 566501 | AA151917 | 2.97 | 7.28 | 2.45 | 2.15 | 1.50 | 2.68 | 1.02 | 3.10 | 4.26 | 2 |
| 584 | 745206 | AA626864 | 0.53 | 1.30 | 2.45 | 1.24 | 2.32 | 2.05 | 2.52 | 3.50 | 3.07 | 2 |
| 585 | 785890 | AA449362 | 6.28 | 15.39 | 2.45 | 3.01 | 2.79 | 1.45 | 1.12 | 2.10 | 4.21 | 2 |
| 586 | 358046 | W94591 | 0.61 | 1.48 | 2.45 | 3.60 | 6.39 | 1.82 | 1.21 | 1.17 | 0.50 | 2 |
| 587 | 583860 | AA101173 | 0.03 | 0.06 | 2.45 | 3.25 | 0.47 | 1.58 | 0.77 | 1.51 | 7.13 | 2 |
| 588 | 795407 | AA453518 | 0.13 | 0.32 | 2.44 | 0.33 | 0.97 | 1.47 | 5.45 | 0.82 | 5.82 | 2 |
| 589 | 1480995 | AA890098 | 5.95 | 14.53 | 2.44 | 2.08 | 2.15 | 1.42 | 2.58 | 3.31 | 3.12 | 2 |
| 590 | 753162 | AA400457 | 2.39 | 5.84 | 2.44 | 3.25 | 3.62 | 0.79 | 1.92 | 2.27 | 2.80 | 2 |
| 591 | 712499 | AA278382 | 2.63 | 6.41 | 2.44 | 1.50 | 3.82 | 2.40 | 3.47 | 1.58 | 1.88 | 2 |
| 592 | 489656 | AA099369 | 0.04 | 0.10 | 2.44 | 4.08 | 1.37 | 2.01 | 1.00 | 0.24 | 5.92 | 2 |
| 593 | 110503 | T89996 | 0.60 | 1.45 | 2.43 | 0.73 | 1.24 | 0.76 | 0.92 | 5.55 | 5.38 | 2 |
| 594 | 197465 | H52048 | 0.35 | 0.85 | 2.43 | 1.53 | 2.49 | 3.94 | 0.91 | 4.24 | 1.47 | 2 |
| 595 | 429729 | AA011685 | 1.61 | 3.91 | 2.42 | 2.40 | 4.68 | 1.51 | 3.19 | 1.84 | 0.93 | 2 |
| 596 | 731371 | AA421047 | 0.03 | 0.07 | 2.42 | 0.99 | 3.84 | 2.29 | 2.95 | 0.33 | 4.12 | 2 |
| 597 | 782585 | AA447527 | 0.48 | 1.17 | 2.42 | 3.45 | 1.49 | 5.47 | 1.78 | 1.44 | 0.90 | 2 |
| 598 | 811010 | AA485385 | 0.06 | 0.14 | 2.42 | 1.99 | 2.41 | 3.45 | 1.97 | 0.58 | 4.13 | 2 |
| 599 | 743445 | AA609365 | 3.21 | 7.76 | 2.42 | 2.19 | 3.40 | 1.76 | 1.31 | 2.53 | 3.33 | 2 |
| 600 | 307328 | N93470 | 0.03 | 0.07 | 2.42 | 5.00 | 3.80 | 0.67 | 1.74 | 2.14 | 1.15 | 2 |
| 601 | 297439 | W03687 | 24.42 | 59.00 | 2.42 | 2.70 | 4.38 | 0.99 | 4.78 | 1.41 | 0.25 | 2 |
| 602 | 245320 | N53458 | 1.20 | 2.89 | 2.41 | 4.58 | 3.25 | 2.53 | 2.71 | 0.66 | 0.75 | 2 |
| 603 | 140057 | R85983 | 0.75 | 1.82 | 2.41 | 1.19 | 2.77 | 2.50 | 3.00 | 1.88 | 3.13 | 2 |
| 604 | 433294 | AA699707 | 1.60 | 3.85 | 2.41 | 1.23 | 3.14 | 1.87 | 1.24 | 4.06 | 2.93 | 2 |
| 605 | 812227 | AA455369 | 4.47 | 10.79 | 2.41 | 3.67 | 5.67 | 2.04 | 1.25 | 0.84 | 0.98 | 2 |
| 606 | 1560977 | AA975010 | 1.07 | 2.57 | 2.41 | 2.29 | 2.67 | 3.01 | 3.69 | 1.52 | 1.27 | 2 |
| 607 | 283379 | N52767 | 0.04 | 0.10 | 2.41 | 4.36 | 4.19 | 2.02 | 0.77 | 0.87 | 2.25 | 2 |
| 608 | 85195 | T71360 | 0.02 | 0.05 | 2.41 | 4.76 | 0.45 | 0.29 | 0.45 | 8.06 | 0.45 | 2 |
| 609 | 813310 | AA455164 | 3.54 | 8.52 | 2.40 | 2.76 | 4.07 | 1.41 | 2.17 | 3.03 | 0.99 | 2 |
| 610 | 222157 | H85345 | 20.24 | 48.65 | 2.40 | 1.65 | 2.06 | 3.15 | 5.03 | 1.84 | 0.69 | 2 |
| 611 | 489098 | AA056534 | 0.42 | 1.02 | 2.40 | 2.31 | 3.10 | 1.61 | 1.66 | 4.37 | 1.35 | 2 |
| 612 | 1456776 | AA863314 | 1.81 | 4.35 | 2.40 | 2.09 | 0.93 | 1.72 | 2.47 | 3.18 | 4.01 | 2 |
| 613 | 1585549 | AA976714 | 5.57 | 13.37 | 2.40 | 2.02 | 2.16 | 2.06 | 3.84 | 3.19 | 1.14 | 2 |
| 614 | 197787 | R93727 | 1.09 | 2.61 | 2.40 | 4.02 | 3.00 | 1.99 | 2.54 | 1.47 | 1.39 | 2 |
| 615 | 731272 | AA420993 | 3.88 | 9.29 | 2.40 | 5.00 | 3.67 | 1.60 | 1.07 | 1.12 | 1.93 | 2 |
| 616 | 501431 | AA115300 | 1.00 | 2.39 | 2.39 | 3.92 | 3.62 | 1.47 | 1.73 | 1.61 | 2.01 | 2 |
| 617 | 415583 | W94316 | 0.76 | 1.81 | 2.39 | 1.52 | 1.61 | 3.50 | 2.18 | 4.08 | 1.46 | 2 |
| 618 | 399390 | AA732832 | 0.10 | 0.24 | 2.39 | 3.36 | 2.17 | 1.55 | 5.29 | 0.90 | 1.06 | 2 |
| 619 | 767981 | AA418828 | 6.24 | 14.88 | 2.39 | 3.46 | 6.52 | 1.04 | 1.41 | 1.37 | 0.52 | 2 |
| 620 | 42325 | R61796 | 18.69 | 44.60 | 2.39 | 5.17 | 3.13 | 2.96 | 0.88 | 0.70 | 1.47 | 2 |
| 621 | 428411 | AA005386 | 1.75 | 4.16 | 2.38 | 1.69 | 3.32 | 2.18 | 3.03 | 2.42 | 1.66 | 2 |
| 622 | 272578 | N35907 | 11.37 | 27.10 | 2.38 | 2.51 | 4.51 | 3.56 | 2.49 | 0.50 | 0.73 | 2 |
| 623 | 127063 | R07891 | 1.23 | 2.93 | 2.38 | 3.82 | 3.35 | 1.65 | 1.41 | 2.81 | 1.24 | 2 |
| 624 | 796366 | AA456135 | 12.87 | 30.59 | 2.38 | 1.77 | 3.33 | 2.54 | 4.28 | 1.43 | 0.91 | 2 |
| 625 | 795827 | AA481506 | 0.03 | 0.07 | 2.37 | 0.67 | 4.90 | 0.75 | 5.01 | 1.77 | 1.15 | 2 |
| 626 | 1602264 | AA988920 | 1.50 | 3.56 | 2.37 | 2.49 | 3.07 | 2.31 | 3.14 | 1.72 | 1.48 | 2 |
| 627 | 487959 | AA054585 | 0.20 | 0.47 | 2.37 | 1.64 | 2.34 | 3.45 | 3.39 | 0.66 | 2.73 | 2 |
| 628 | 1460391 | AA883612 | 0.78 | 1.84 | 2.37 | 1.84 | 3.09 | 1.87 | 3.81 | 2.50 | 1.09 | 2 |
| 629 | 22700 | R43566 | 1.08 | 2.56 | 2.37 | 1.06 | 1.93 | 3.22 | 1.78 | 4.47 | 1.74 | 2 |
| 630 | 432581 | AA699390 | 7.05 | 16.64 | 2.36 | 4.09 | 4.25 | 2.31 | 1.16 | 1.43 | 0.93 | 2 |
| 631 | 757222 | AA496149 | 2.81 | 6.63 | 2.36 | 1.94 | 3.55 | 0.84 | 0.90 | 1.70 | 5.24 | 2 |
| 632 | 244896 | N54512 | 0.28 | 0.67 | 2.36 | 1.88 | 3.50 | 3.02 | 1.59 | 1.37 | 2.79 | 2 |
| 633 | 413300 | AA772497 | 0.36 | 0.84 | 2.36 | 1.84 | 3.27 | 2.31 | 2.33 | 1.24 | 3.15 | 2 |
| 634 | 265443 | N66393 | 1.60 | 3.77 | 2.36 | 1.65 | 5.16 | 1.06 | 1.43 | 3.15 | 1.69 | 2 |
| 635 | 825857 | AA504783 | 1.54 | 3.61 | 2.35 | 4.14 | 3.65 | 1.91 | 2.15 | 0.83 | 1.43 | 2 |
| 636 | 284938 | N21237 | 4.33 | 10.16 | 2.35 | 1.58 | 4.36 | 1.89 | 4.01 | 1.75 | 0.50 | 2 |
| 637 | 43709 | H05734 | 0.57 | 1.34 | 2.35 | 4.38 | 2.28 | 1.17 | 0.79 | 0.75 | 4.71 | 2 |
| 638 | 41720 | R54105 | 0.09 | 0.21 | 2.35 | 2.89 | 0.26 | 3.80 | 0.62 | 4.07 | 2.43 | 2 |
| 639 | 299679 | W05628 | 0.81 | 1.89 | 2.34 | 2.08 | 3.24 | 1.37 | 1.91 | 4.73 | 0.72 | 2 |
| 640 | 731180 | AA417246 | 0.64 | 1.51 | 2.34 | 2.08 | 1.24 | 2.39 | 1.15 | 3.46 | 3.75 | 2 |
| 641 | 810284 | AA463932 | 0.60 | 1.40 | 2.34 | 1.52 | 1.71 | 1.79 | 3.21 | 3.01 | 2.78 | 2 |
| 642 | 898055 | AA598958 | 0.05 | 0.12 | 2.34 | 3.49 | 2.31 | 2.89 | 3.16 | 0.12 | 2.05 | 2 |
| 643 | 1240220 | AA788970 | 4.63 | 10.81 | 2.33 | 3.43 | 7.25 | 0.74 | 0.71 | 0.91 | 0.97 | 2 |
| 644 | 242037 | H93328 | 0.38 | 0.89 | 2.33 | 3.07 | 2.13 | 0.85 | 1.88 | 4.32 | 1.76 | 2 |
| 645 | 470914 | AA032084 | 13.04 | 30.41 | 2.33 | 2.36 | 3.53 | 1.24 | 2.33 | 3.03 | 1.50 | 2 |
| 646 | 38260 | R49482 | 0.70 | 1.64 | 2.33 | 1.90 | 0.47 | 0.87 | 0.59 | 5.53 | 4.59 | 2 |
| 647 | 50250 | H17800 | 0.81 | 1.89 | 2.32 | 3.30 | 3.08 | 2.73 | 2.02 | 1.50 | 1.32 | 2 |
| 648 | 743217 | AA400128 | 1.85 | 4.30 | 2.32 | 1.78 | 3.31 | 2.05 | 2.70 | 3.18 | 0.92 | 2 |
| 649 | 290841 | N71982 | 4.59 | 10.65 | 2.32 | 3.15 | 3.16 | 0.87 | 2.32 | 1.70 | 2.71 | 2 |
| 650 | 814595 | AA480906 | 5.94 | 13.75 | 2.32 | 1.14 | 2.10 | 1.47 | 3.01 | 4.67 | 1.50 | 2 |
| 651 | 240766 | H80215 | 2.74 | 6.35 | 2.32 | 1.73 | 3.33 | 3.46 | 2.02 | 1.79 | 1.56 | 2 |
| 652 | 34889 | R44447 | 2.35 | 5.45 | 2.31 | 1.25 | 1.34 | 1.59 | 3.79 | 2.55 | 3.37 | 2 |
| 653 | 51263 | H18838 | 0.06 | 0.14 | 2.31 | 4.10 | 1.12 | 1.78 | 1.85 | 1.37 | 3.63 | 2 |
| 654 | 810209 | AA464522 | 1.10 | 2.54 | 2.30 | 1.35 | 0.96 | 5.47 | 1.37 | 3.40 | 1.28 | 2 |
| 655 | 795831 | AA461508 | 0.07 | 0.17 | 2.30 | 1.39 | 2.39 | 3.59 | 5.16 | 0.46 | 0.83 | 2 |
| 656 | 745138 | AA626698 | 12.76 | 29.36 | 2.30 | 3.25 | 2.34 | 3.04 | 2.67 | 1.74 | 0.77 | 2 |
| 657 | 489076 | AA057195 | 0.02 | 0.05 | 2.30 | 1.88 | 3.30 | 2.10 | 5.47 | 0.55 | 0.48 | 2 |
| 658 | 344108 | W73781 | 3.42 | 7.83 | 2.29 | 4.00 | 3.17 | 2.56 | 1.61 | 0.98 | 1.43 | 2 |
| 659 | 727192 | AA293819 | 12.78 | 29.26 | 2.29 | 2.09 | 0.89 | 2.24 | 3.41 | 3.56 | 1.55 | 2 |
| 660 | 41541 | R52789 | 4.22 | 9.64 | 2.29 | 2.19 | 3.57 | 3.59 | 1.28 | 1.49 | 1.60 | 2 |
| 661 | 795353 | AA453487 | 4.31 | 9.88 | 2.29 | 1.68 | 4.15 | 1.50 | 4.56 | 0.88 | 0.94 | 2 |
| 662 | 40400 | R52088 | 0.31 | 0.71 | 2.28 | 2.41 | 0.86 | 1.19 | 0.31 | 4.68 | 4.26 | 2 |
| 663 | 48330 | H14949 | 0.17 | 0.38 | 2.28 | 1.85 | 3.18 | 2.62 | 2.50 | 3.07 | 0.48 | 2 |
| 664 | 769600 | AA425900 | 0.09 | 0.20 | 2.28 | 3.18 | 0.12 | 2.08 | 2.75 | 5.09 | 0.47 | 2 |
| 665 | 112885 | T87139 | 2.61 | 5.95 | 2.28 | 1.59 | 3.50 | 3.29 | 1.78 | 1.99 | 1.51 | 2 |
| 666 | 382795 | AA018663 | 0.06 | 0.13 | 2.28 | 2.04 | 6.08 | 0.18 | 3.14 | 0.18 | 2.05 | 2 |
| 667 | 384397 | AA708676 | 0.46 | 1.05 | 2.27 | 1.47 | 3.59 | 1.54 | 1.98 | 3.17 | 1.91 | 2 |
| 668 | 26588 | R39111 | 0.74 | 1.68 | 2.27 | 3.15 | 0.55 | 0.70 | 2.64 | 1.53 | 5.05 | 2 |
| 669 | 199228 | R95811 | 0.58 | 1.27 | 2.27 | 1.69 | 3.64 | 3.07 | 1.93 | 1.63 | 1.65 | 2 |
| 670 | 41070 | R56100 | 0.03 | 0.07 | 2.27 | 1.48 | 2.18 | 0.33 | 1.19 | 4.15 | 4.27 | 2 |
| 671 | 51103 | H19217 | 0.37 | 0.83 | 2.27 | 0.74 | 4.79 | 1.44 | 1.55 | 3.13 | 1.94 | 2 |
| 672 | 346552 | W74377 | 0.02 | 0.04 | 2.27 | 3.52 | 2.57 | 3.03 | 2.05 | 0.61 | 1.81 | 2 |
| 673 | 624786 | AA181995 | 4.34 | 9.82 | 2.26 | 3.02 | 3.29 | 1.52 | 2.48 | 1.77 | 1.51 | 2 |
| 674 | 626718 | AA191548 | 17.62 | 39.85 | 2.26 | 3.68 | 3.59 | 0.94 | 2.27 | 1.42 | 1.67 | 2 |
| 675 | 785760 | AA449688 | 0.02 | 0.04 | 2.26 | 3.56 | 3.49 | 2.71 | 2.58 | 0.71 | 0.53 | 2 |
| 676 | 345145 | W72231 | 3.57 | 8.07 | 2.26 | 3.73 | 3.52 | 2.16 | 1.92 | 0.60 | 1.62 | 2 |
| 677 | 48620 | H10036 | 15.87 | 35.82 | 2.26 | 0.96 | 1.62 | 1.52 | 1.49 | 3.82 | 4.13 | 2 |
| 678 | 811832 | AA463627 | 0.05 | 0.11 | 2.25 | 0.87 | 0.21 | 2.33 | 0.21 | 5.03 | 4.87 | 2 |
| 679 | 49141 | H16554 | 0.19 | 0.44 | 2.25 | 1.32 | 2.11 | 3.19 | 1.73 | 1.38 | 3.78 | 2 |
| 680 | 823691 | AA489752 | 2.53 | 5.67 | 2.24 | 3.20 | 3.06 | 1.94 | 1.36 | 1.62 | 2.28 | 2 |
| 681 | 754127 | AA478627 | 3.54 | 7.92 | 2.23 | 2.21 | 5.87 | 0.66 | 3.94 | 0.37 | 0.36 | 2 |
| 682 | 1481609 | AA883800 | 0.50 | 1.11 | 2.23 | 1.70 | 1.34 | 0.96 | 3.17 | 4.44 | 1.75 | 2 |
| 683 | 45099 | H05140 | 0.32 | 0.70 | 2.22 | 1.86 | 4.17 | 1.42 | 3.53 | 0.72 | 1.63 | 2 |
| 684 | 232912 | H72643 | 21.80 | 48.25 | 2.21 | 3.16 | 3.03 | 1.94 | 1.77 | 1.87 | 1.72 | 2 |
| 685 | 135789 | R33353 | 0.11 | 0.25 | 2.21 | 1.97 | 3.06 | 2.59 | 3.48 | 0.79 | 1.35 | 2 |
| 686 | 795308 | AA454149 | 0.10 | 0.23 | 2.21 | 3.92 | 3.22 | 1.18 | 2.96 | 1.72 | 0.26 | 2 |
| 687 | 449270 | AA777696 | 0.06 | 0.13 | 2.21 | 2.46 | 1.00 | 3.23 | 2.25 | 1.21 | 3.10 | 2 |
| 688 | 898195 | AA598587 | 1.71 | 3.77 | 2.20 | 1.65 | 3.01 | 0.59 | 2.16 | 2.77 | 3.05 | 2 |
| 689 | 131073 | R23241 | 4.56 | 10.05 | 2.20 | 1.61 | 1.24 | 1.02 | 5.17 | 0.91 | 3.26 | 2 |
| 690 | 233277 | H77494 | 0.98 | 2.16 | 2.20 | 1.77 | 3.15 | 3.31 | 2.10 | 1.98 | 0.90 | 2 |
| 691 | 243800 | N39338 | 1.49 | 3.27 | 2.20 | 2.38 | 1.42 | 1.49 | 3.06 | 1.59 | 3.27 | 2 |
| 692 | 782541 | AA448486 | 0.13 | 0.29 | 2.20 | 1.26 | 3.11 | 6.76 | 0.80 | 0.20 | 1.07 | 2 |
| 693 | 813841 | AA447744 | 0.48 | 1.06 | 2.20 | 1.94 | 2.03 | 1.19 | 1.36 | 3.30 | 3.35 | 2 |
| 694 | 126568 | R06862 | 3.87 | 8.47 | 2.19 | 2.26 | 3.85 | 3.24 | 1.57 | 1.12 | 1.09 | 2 |
| 695 | 345553 | W73889 | 0.05 | 0.11 | 2.19 | 1.87 | 1.73 | 3.63 | 0.20 | 0.20 | 5.50 | 2 |
| 696 | 51947 | H24313 | 2.05 | 4.49 | 2.19 | 1.10 | 1.74 | 2.61 | 3.42 | 3.28 | 0.98 | 2 |
| 697 | 40010 | R54038 | 0.09 | 0.21 | 2.19 | 0.84 | 0.11 | 3.51 | 0.13 | 1.93 | 6.60 | 2 |
| 698 | 383188 | AA074224 | 0.29 | 0.64 | 2.18 | 1.56 | 3.14 | 2.20 | 0.53 | 4.87 | 0.81 | 2 |
| 699 | 73531 | T55839 | 0.05 | 0.11 | 2.18 | 3.98 | 1.50 | 0.27 | 3.22 | 1.35 | 2.76 | 2 |
| 700 | 788020 | AA418748 | 3.05 | 6.63 | 2.18 | 0.48 | 0.82 | 6.02 | 3.04 | 0.78 | 1.93 | 2 |
| 701 | 33860 | R44840 | 6.15 | 13.38 | 2.18 | 2.11 | 3.23 | 3.65 | 1.29 | 1.24 | 1.53 | 2 |
| 702 | 46166 | H09076 | 3.52 | 7.66 | 2.17 | 4.03 | 4.62 | 2.45 | 1.01 | 0.17 | 0.77 | 2 |
| 703 | 283191 | N51362 | 2.72 | 5.90 | 2.17 | 3.17 | 3.06 | 0.65 | 2.60 | 0.77 | 2.79 | 2 |
| 704 | 647985 | AA207105 | 11.41 | 24.76 | 2.17 | 1.43 | 3.18 | 0.73 | 2.68 | 3.94 | 1.06 | 2 |
| 705 | 491001 | AA138710 | 5.71 | 12.38 | 2.17 | 1.28 | 0.93 | 1.27 | 1.26 | 4.03 | 4.23 | 2 |
| 708 | 1461161 | AA868038 | 1.08 | 2.34 | 2.16 | 1.67 | 1.35 | 3.92 | 3.45 | 1.19 | 1.39 | 2 |
| 707 | 757503 | AA426380 | 1.95 | 4.21 | 2.16 | 1.68 | 4.53 | 1.35 | 3.60 | 1.13 | 0.68 | 2 |
| 708 | 280375 | N47113 | 1.16 | 2.51 | 2.16 | 4.65 | 3.70 | 1.70 | 0.96 | 0.72 | 1.23 | 2 |
| 709 | 363916 | AA021247 | 1.43 | 3.09 | 2.16 | 1.68 | 3.74 | 1.81 | 3.80 | 0.98 | 0.92 | 2 |
| 710 | 121729 | T98001 | 0.73 | 1.58 | 2.15 | 1.34 | 3.07 | 1.77 | 1.30 | 2.36 | 3.09 | 2 |
| 711 | 685801 | AA262080 | 1.29 | 2.77 | 2.15 | 1.58 | 0.79 | 0.98 | 5.28 | 1.08 | 3.20 | 2 |
| 712 | 207370 | H58834 | 0.13 | 0.27 | 2.15 | 1.14 | 1.36 | 1.33 | 3.92 | 1.95 | 3.18 | 2 |
| 713 | 202795 | H53964 | 2.15 | 4.60 | 2.14 | 1.35 | 3.31 | 3.06 | 2.27 | 1.88 | 0.96 | 2 |
| 714 | 826138 | AA521337 | 0.01 | 0.02 | 2.14 | 4.28 | 0.94 | 4.27 | 1.46 | 0.94 | 0.94 | 2 |
| 715 | 322461 | W15305 | 2.11 | 4.50 | 2.13 | 3.57 | 3.32 | 2.76 | 0.71 | 1.30 | 1.13 | 2 |
| 716 | 188232 | H45711 | 0.61 | 1.31 | 2.13 | 3.34 | 1.13 | 1.34 | 1.68 | 1.16 | 4.14 | 2 |
| 717 | 197525 | H52001 | 2.60 | 5.54 | 2.13 | 2.40 | 3.78 | 3.35 | 2.45 | 0.34 | 0.47 | 2 |
| 718 | 460279 | AA677629 | 0.68 | 1.44 | 2.13 | 2.50 | 3.41 | 1.76 | 3.41 | 1.02 | 0.69 | 2 |
| 719 | 245444 | N53492 | 0.53 | 1.12 | 2.13 | 1.10 | 2.23 | 1.40 | 3.97 | 3.07 | 1.03 | 2 |
| 720 | 83029 | T67807 | 1.42 | 3.02 | 2.13 | 0.99 | 3.50 | 3.33 | 2.50 | 0.59 | 1.88 | 2 |
| 721 | 276871 | N39426 | 0.05 | 0.11 | 2.13 | 8.86 | 0.19 | 1.08 | 0.19 | 1.11 | 3.34 | 2 |
| 722 | 417059 | W87801 | 0.12 | 0.25 | 2.13 | 0.40 | 4.34 | 0.31 | 5.94 | 1.68 | 0.08 | 2 |
| 723 | 294255 | N99839 | 13.11 | 27.87 | 2.13 | 1.47 | 3.12 | 3.23 | 1.23 | 1.67 | 2.04 | 2 |
| 724 | 243700 | N45083 | 1.34 | 2.84 | 2.13 | 1.35 | 3.02 | 1.62 | 1.40 | 4.42 | 0.94 | 2 |
| 725 | 285040 | N63375 | 5.95 | 12.63 | 2.12 | 3.18 | 3.00 | 0.85 | 1.26 | 1.60 | 2.84 | 2 |
| 726 | 73252 | T56013 | 1.33 | 2.82 | 2.12 | 1.70 | 3.05 | 1.47 | 4.55 | 0.92 | 1.02 | 2 |
| 727 | 743465 | AA609385 | 1.79 | 3.78 | 2.11 | 0.90 | 1.15 | 3.08 | 0.92 | 0.92 | 5.71 | 2 |
| 728 | 154485 | R54850 | 4.31 | 9.09 | 2.11 | 3.15 | 3.51 | 2.17 | 1.16 | 1.40 | 1.27 | 2 |
| 729 | 814780 | AA455227 | 0.13 | 0.27 | 2.10 | 3.09 | 0.96 | 3.49 | 2.09 | 0.98 | 2.01 | 2 |
| 730 | 322005 | W37418 | 0.91 | 1.90 | 2.10 | 1.29 | 1.12 | 3.41 | 1.85 | 1.43 | 3.49 | 2 |
| 731 | 416945 | W87710 | 3.08 | 6.46 | 2.10 | 1.84 | 3.31 | 3.19 | 1.59 | 1.17 | 1.68 | 2 |
| 732 | 624429 | AA182680 | 6.29 | 13.17 | 2.10 | 1.20 | 3.14 | 1.32 | 2.68 | 3.20 | 1.03 | 2 |
| 733 | 1031844 | AA609686 | 0.08 | 0.16 | 2.09 | 0.17 | 1.06 | 0.74 | 4.67 | 1.56 | 4.35 | 2 |
| 734 | 345176 | W72263 | 0.18 | 0.37 | 2.09 | 1.24 | 1.24 | 1.45 | 4.29 | 4.03 | 0.28 | 2 |
| 735 | 878652 | AA670200 | 1.48 | 3.08 | 2.09 | 1.94 | 3.20 | 1.64 | 3.65 | 0.99 | 1.11 | 2 |
| 736 | 366511 | AA026609 | 4.79 | 9.99 | 2.09 | 1.37 | 3.02 | 3.06 | 1.55 | 1.49 | 2.04 | 2 |
| 737 | 235173 | H73013 | 1.44 | 2.99 | 2.08 | 1.48 | 3.27 | 3.55 | 0.87 | 1.39 | 1.95 | 2 |
| 738 | 296448 | N74823 | 0.06 | 0.13 | 2.08 | 2.84 | 0.16 | 0.56 | 3.12 | 4.93 | 0.84 | 2 |
| 739 | 357284 | W93585 | 1.11 | 2.28 | 2.06 | 2.07 | 3.92 | 0.92 | 3.60 | 1.38 | 0.44 | 2 |
| 740 | 39799 | R52640 | 0.33 | 0.67 | 2.05 | 1.08 | 3.65 | 1.82 | 0.25 | 0.28 | 5.19 | 2 |
| 741 | 462957 | AA882425 | 1.20 | 2.45 | 2.04 | 0.92 | 3.31 | 1.69 | 1.59 | 3.62 | 1.14 | 2 |
| 742 | 292770 | N63846 | 0.07 | 0.15 | 2.04 | 4.41 | 2.53 | 0.14 | 0.63 | 1.16 | 3.40 | 2 |
| 743 | 195835 | R89317 | 0.26 | 0.53 | 2.04 | 3.70 | 3.56 | 1.95 | 1.90 | 0.32 | 0.80 | 2 |
| 744 | 583673 | AA101299 | 0.58 | 1.19 | 2.03 | 2.13 | 3.21 | 3.13 | 2.60 | 0.64 | 0.50 | 2 |
| 745 | 795522 | AA454218 | 0.05 | 0.10 | 2.03 | 0.66 | 0.20 | 0.96 | 3.23 | 5.47 | 1.68 | 2 |
| 746 | 42035 | R59068 | 0.19 | 0.39 | 2.03 | 1.37 | 1.12 | 3.98 | 1.13 | 3.33 | 1.27 | 2 |
| 747 | 280412 | N47208 | 0.02 | 0.05 | 2.03 | 4.97 | 1.05 | 0.57 | 0.84 | 0.40 | 4.35 | 2 |
| 748 | 700302 | AA283893 | 0.04 | 0.09 | 2.03 | 1.01 | 0.21 | 3.77 | 1.83 | 1.37 | 3.99 | 2 |
| 749 | 40082 | R52530 | 0.33 | 0.66 | 2.02 | 3.25 | 0.81 | 1.78 | 0.58 | 1.83 | 3.88 | 2 |
| 750 | 1488109 | AA936783 | 112.27 | 226.02 | 2.01 | 1.69 | 3.11 | 1.86 | 3.12 | 1.33 | 0.97 | 2 |
| 751 | 415906 | W86196 | 0.27 | 0.55 | 2.01 | 1.00 | 3.06 | 1.36 | 1.47 | 2.10 | 3.06 | 2 |
| 752 | 399421 | AA733177 | 2.08 | 4.18 | 2.01 | 0.73 | 0.73 | 1.08 | 3.17 | 3.58 | 2.77 | 2 |
| 753 | 744962 | AA625907 | 10.80 | 21.89 | 2.01 | 1.77 | 1.69 | 3.05 | 3.01 | 1.55 | 0.99 | 2 |
| 754 | 415111 | W93147 | 0.65 | 1.30 | 2.01 | 1.54 | 3.53 | 3.51 | 1.34 | 1.36 | 0.77 | 2 |
| 755 | 811046 | AA485427 | 0.08 | 0.16 | 2.01 | 1.48 | 0.12 | 1.12 | 4.05 | 0.90 | 4.37 | 2 |
| 756 | 590640 | AA158035 | 0.14 | 0.27 | 2.01 | 0.92 | 3.66 | 0.93 | 0.61 | 2.31 | 3.41 | 2 |
| 757 | 588053 | AA135001 | 2.74 | 5.47 | 1.99 | 0.61 | 0.28 | 1.31 | 4.80 | 4.27 | 0.70 | 2 |
| 758 | 278496 | N66135 | 2.33 | 4.63 | 1.99 | 1.28 | 3.38 | 1.63 | 3.00 | 1.74 | 0.87 | 2 |
| 759 | 32982 | R43910 | 0.33 | 0.65 | 1.98 | 0.56 | 4.06 | 1.99 | 0.92 | 3.70 | 0.67 | 2 |
| 760 | 878785 | AA870411 | 13.20 | 26.15 | 1.98 | 1.66 | 3.55 | 1.14 | 3.73 | 1.31 | 0.49 | 2 |
| 761 | 827105 | AA190634 | 9.88 | 19.43 | 1.97 | 1.41 | 3.00 | 1.85 | 3.99 | 1.07 | 0.49 | 2 |
| 762 | 42123 | R59621 | 1.76 | 3.45 | 1.96 | 2.41 | 3.44 | 0.45 | 3.67 | 1.34 | 0.42 | 2 |
| 763 | 245547 | N55167 | 2.57 | 5.02 | 1.95 | 3.53 | 4.14 | 1.49 | 0.91 | 0.44 | 1.22 | 2 |
| 764 | 752704 | AA417792 | 0.13 | 0.25 | 1.95 | 1.25 | 3.84 | 1.06 | 1.57 | 0.60 | 3.40 | 2 |
| 765 | 48921 | H10228 | 3.50 | 6.82 | 1.95 | 0.96 | 3.61 | 1.32 | 3.38 | 1.26 | 1.16 | 2 |
| 766 | 1031309 | AA809088 | 0.51 | 0.99 | 1.95 | 3.04 | 3.06 | 0.98 | 1.89 | 1.68 | 1.04 | 2 |
| 767 | 376043 | AA040265 | 0.07 | 0.13 | 1.94 | 0.15 | 3.60 | 2.65 | 0.63 | 0.15 | 4.47 | 2 |
| 768 | 51373 | H24006 | 0.13 | 0.26 | 1.94 | 2.30 | 1.24 | 3.70 | 3.77 | 0.56 | 0.07 | 2 |
| 769 | 361363 | AA017706 | 5.53 | 10.65 | 1.93 | 1.54 | 0.87 | 1.45 | 1.35 | 3.22 | 3.15 | 2 |
| 770 | 451121 | AA704537 | 0.59 | 1.13 | 1.92 | 0.78 | 1.30 | 1.49 | 1.78 | 3.10 | 3.11 | 2 |
| 771 | 769933 | AA430506 | 0.52 | 0.99 | 1.92 | 1.31 | 1.88 | 0.78 | 0.75 | 3.17 | 3.66 | 2 |
| 772 | 782826 | AA448257 | 0.06 | 0.12 | 1.92 | 0.80 | 0.16 | 1.67 | 0.16 | 3.99 | 4.76 | 2 |
| 773 | 841281 | AA487203 | 1.52 | 2.93 | 1.92 | 4.92 | 0.83 | 0.83 | 0.96 | 0.99 | 3.01 | 2 |
| 774 | 610883 | AA172188 | 2.84 | 5.46 | 1.92 | 1.96 | 3.63 | 1.18 | 3.76 | 0.32 | 0.68 | 2 |
| 775 | 613730 | AA453783 | 2.20 | 4.23 | 1.92 | 3.26 | 3.09 | 0.62 | 0.89 | 1.23 | 2.43 | 2 |
| 776 | 610996 | AA485351 | 0.03 | 0.05 | 1.92 | 0.60 | 0.39 | 4.59 | 5.15 | 0.39 | 0.39 | 2 |
| 777 | 841666 | AA487561 | 5.91 | 11.33 | 1.92 | 1.22 | 0.65 | 1.41 | 3.56 | 0.92 | 3.71 | 2 |
| 778 | 277112 | N39611 | 0.73 | 1.38 | 1.91 | 2.14 | 3.20 | 1.12 | 3.17 | 0.78 | 1.04 | 2 |
| 779 | 530608 | AA071089 | 0.05 | 0.10 | 1.90 | 0.18 | 0.16 | 0.54 | 1.35 | 3.59 | 5.59 | 2 |
| 780 | 208570 | H83223 | 0.59 | 1.10 | 1.69 | 0.51 | 0.48 | 2.48 | 3.11 | 3.59 | 1.14 | 2 |
| 781 | 131887 | R25166 | 2.12 | 3.98 | 1.88 | 3.19 | 1.11 | 1.57 | 0.87 | 1.04 | 3.49 | 2 |
| 782 | 341978 | W61381 | 1.28 | 2.41 | 1.88 | 3.07 | 3.06 | 1.81 | 1.31 | 0.70 | 1.31 | 2 |
| 783 | 461727 | AA682293 | 1.49 | 2.80 | 1.88 | 3.10 | 0.77 | 2.34 | 3.24 | 0.89 | 0.92 | 2 |
| 784 | 815550 | AA458818 | 3.03 | 5.67 | 1.87 | 0.83 | 0.89 | 1.54 | 0.50 | 4.16 | 3.30 | 2 |
| 785 | 33999 | R44538 | 7.22 | 13.49 | 1.87 | 0.98 | 3.01 | 1.39 | 4.58 | 0.80 | 0.48 | 2 |
| 786 | 34041 | R44754 | 0.12 | 0.23 | 1.87 | 0.95 | 3.90 | 1.51 | 1.20 | 0.08 | 3.55 | 2 |
| 787 | 743189 | AA401434 | 0.04 | 0.08 | 1.86 | 2.05 | 3.30 | 1.81 | 3.55 | 0.24 | 0.23 | 2 |
| 788 | 247194 | N54036 | 0.36 | 0.66 | 1.86 | 1.09 | 3.23 | 1.26 | 3.14 | 1.38 | 1.06 | 2 |
| 789 | 183771 | H44448 | 1.91 | 3.56 | 1.86 | 1.23 | 3.04 | 1.22 | 3.07 | 1.82 | 0.79 | 2 |
| 790 | 491764 | AA150502 | 0.08 | 0.14 | 1.86 | 3.80 | 3.09 | 1.20 | 1.47 | 1.48 | 0.13 | 2 |
| 791 | 782594 | AA447531 | 0.02 | 0.04 | 1.85 | 3.26 | 3.78 | 2.14 | 1.09 | 0.42 | 0.42 | 2 |
| 792 | 471859 | AA035144 | 0.03 | 0.05 | 1.85 | 3.10 | 5.05 | 0.57 | 0.47 | 1.55 | 0.39 | 2 |
| 793 | 276712 | N34895 | 0.13 | 0.23 | 1.85 | 0.30 | 0.39 | 5.27 | 1.75 | 0.10 | 3.28 | 2 |
| 794 | 44154 | H06154 | 1.53 | 2.83 | 1.85 | 2.02 | 3.26 | 0.79 | 3.22 | 1.20 | 0.59 | 2 |
| 795 | 261274 | H98255 | 2.71 | 5.00 | 1.84 | 1.16 | 3.49 | 1.61 | 3.99 | 0.39 | 0.42 | 2 |
| 796 | 839081 | AA487608 | 11.79 | 21.75 | 1.84 | 3.03 | 3.73 | 0.71 | 2.01 | 0.60 | 0.98 | 2 |
| 797 | 502625 | AA134578 | 0.88 | 1.81 | 1.84 | 4.66 | 0.80 | 0.64 | 0.68 | 0.19 | 4.08 | 2 |
| 798 | 77577 | T58932 | 0.29 | 0.52 | 1.83 | 1.41 | 3.32 | 1.19 | 3.24 | 1.13 | 0.68 | 2 |
| 799 | 839829 | AA489782 | 0.23 | 0.41 | 1.82 | 1.28 | 3.14 | 1.17 | 1.64 | 3.17 | 0.53 | 2 |
| 800 | 53091 | R15784 | 0.11 | 0.20 | 1.82 | 1.57 | 0.53 | 0.62 | 0.77 | 4.18 | 3.24 | 2 |
| 801 | 358531 | W96134 | 2.78 | 5.05 | 1.82 | 3.97 | 0.53 | 1.15 | 0.95 | 0.69 | 3.60 | 2 |
| 802 | 32339 | R42823 | 0.14 | 0.24 | 1.80 | 0.81 | 0.47 | 3.98 | 3.10 | 0.42 | 2.00 | 2 |
| 803 | 417688 | W89074 | 0.20 | 0.35 | 1.80 | 1.48 | 0.57 | 0.05 | 1.03 | 3.31 | 4.34 | 2 |
| 804 | 1435339 | AA857748 | 0.04 | 0.07 | 1.79 | 0.22 | 3.54 | 0.73 | 1.30 | 0.25 | 4.72 | 2 |
| 805 | 128973 | R10301 | 0.05 | 0.09 | 1.75 | 0.93 | 4.27 | 3.36 | 1.30 | 0.20 | 0.43 | 2 |
| 806 | 361323 | AA017544 | 2.04 | 3.57 | 1.75 | 0.84 | 1.00 | 3.73 | 3.19 | 0.89 | 0.86 | 2 |
| 807 | 566440 | AA148862 | 0.05 | 0.09 | 1.75 | 0.31 | 0.77 | 1.71 | 3.44 | 0.20 | 4.04 | 2 |
| 808 | 857196 | AA773139 | 0.46 | 0.79 | 1.74 | 1.59 | 3.02 | 0.94 | 0.48 | 0.90 | 3.53 | 2 |
| 809 | 588958 | AA130984 | 0.03 | 0.06 | 1.71 | 6.02 | 0.31 | 3.02 | 0.31 | 0.31 | 0.31 | 2 |
| 810 | 741891 | AA401972 | 5.81 | 9.84 | 1.69 | 1.85 | 0.69 | 0.62 | 0.74 | 3.11 | 3.35 | 2 |
| 811 | 810027 | AA455275 | 0.04 | 0.07 | 1.69 | 4.25 | 0.80 | 0.94 | 3.45 | 0.57 | 0.16 | 2 |
| 812 | 811609 | AA455020 | 0.03 | 0.05 | 1.65 | 5.05 | 0.33 | 0.21 | 3.08 | 0.21 | 1.05 | 2 |
| 813 | 361607 | W92431 | 0.21 | 0.35 | 1.83 | 0.45 | 3.01 | 1.77 | 0.79 | 0.33 | 3.44 | 2 |
| 814 | 795538 | AA459851 | 0.10 | 0.16 | 1.60 | 0.95 | 0.10 | 0.51 | 3.09 | 3.13 | 1.83 | 2 |
| 815 | 260168 | N32071 | 0.02 | 0.02 | 1.59 | 0.51 | 0.66 | 0.38 | 3.52 | 0.66 | 3.79 | 2 |
| 816 | 609935 | AA169154 | 0.02 | 0.04 | 1.59 | 0.28 | 4.10 | 0.43 | 3.84 | 0.43 | 0.43 | 2 |
| 817 | 47187 | H10404 | 0.06 | 0.10 | 1.57 | 0.21 | 3.55 | 3.54 | 0.45 | 0.16 | 1.52 | 2 |
| 818 | 810948 | AA459383 | 0.05 | 0.09 | 1.57 | 1.00 | 3.48 | 3.31 | 0.64 | 0.18 | 0.81 | 2 |
| 819 | 490556 | AA100696 | 0.04 | 0.07 | 1.56 | 3.03 | 0.23 | 1.13 | 1.63 | 3.05 | 0.27 | 2 |
| 820 | 769579 | AA425826 | 0.11 | 0.17 | 1.53 | 4.53 | 0.39 | 3.52 | 0.20 | 0.18 | 0.34 | 2 |
| 821 | 454083 | AA676998 | 0.05 | 0.07 | 1.47 | 3.29 | 3.40 | 0.22 | 1.51 | 0.22 | 0.22 | 2 |
| 822 | 52071 | H24350 | 0.27 | 0.39 | 1.47 | 3.02 | 3.31 | 0.69 | 0.81 | 0.31 | 0.67 | 2 |
| 823 | 511066 | AA100296 | 0.03 | 0.04 | 1.35 | 0.33 | 0.48 | 3.30 | 3.34 | 0.33 | 0.33 | 2 |
| 624 | 742818 | AA400482 | 0.24 | 0.32 | 1.33 | 3.06 | 0.05 | 1.14 | 0.23 | 0.40 | 3.07 | 2 |
| 825 | 504464 | AA152178 | 0.06 | 1.88 | 32.02 | 186.97 | 2.01 | 0.17 | 2.61 | 0.17 | 0.17 | 1 |
| 826 | 306420 | N92699 | 0.07 | 1.95 | 26.82 | 2.37 | 0.45 | 0.14 | 0.14 | 155.49 | 2.32 | 1 |
| 827 | 452374 | AA700876 | 3.59 | 80.73 | 22.47 | 0.60 | 1.38 | 132.15 | 0.22 | 0.14 | 0.32 | 1 |
| 828 | 384015 | AA702640 | 0.32 | 5.24 | 16.48 | 2.18 | 2.14 | 2.48 | 0.75 | 91.20 | 0.14 | 1 |
| 829 | 46641 | H10344 | 0.07 | 0.64 | 8.77 | 0.83 | 0.14 | 0.29 | 2.14 | 46.98 | 2.25 | 1 |
| 830 | 156322 | R72618 | 0.08 | 0.61 | 7.80 | 1.37 | 0.78 | 1.76 | 0.14 | 40.15 | 1.39 | 1 |
| 831 | 81413 | T60181 | 0.15 | 0.97 | 6.43 | 0.62 | 0.93 | 1.48 | 1.54 | 0.58 | 33.43 | 1 |
| 832 | 745232 | AA826152 | 0.33 | 1.99 | 6.03 | 2.98 | 2.15 | 24.89 | 1.04 | 2.90 | 2.23 | 1 |
| 833 | 429210 | AA007276 | 0.27 | 1.55 | 5.77 | 0.90 | 0.45 | 0.30 | 1.35 | 0.25 | 31.39 | 1 |
| 834 | 50354 | H17804 | 0.26 | 1.46 | 5.61 | 0.07 | 1.18 | 0.70 | 30.40 | 0.69 | 0.65 | 1 |
| 835 | 364563 | AA022949 | 0.30 | 1.85 | 5.42 | 0.52 | 0.99 | 0.13 | 1.21 | 1.70 | 27.99 | 1 |
| 836 | 488422 | AA044662 | 0.05 | 0.27 | 5.40 | 1.15 | 0.89 | 0.63 | 1.19 | 1.10 | 27.41 | 1 |
| 837 | 50799 | H17630 | 0.30 | 1.59 | 5.36 | 0.82 | 1.21 | 2.14 | 1.43 | 1.89 | 24.70 | 1 |
| 838 | 302180 | N79989 | 0.22 | 1.16 | 5.35 | 2.39 | 0.79 | 1.61 | 1.51 | 24.08 | 1.74 | 1 |
| 839 | 128083 | R09747 | 0.04 | 0.20 | 5.12 | 1.88 | 1.08 | 26.16 | 0.26 | 1.06 | 0.26 | 1 |
| 840 | 25933 | R36969 | 0.07 | 0.37 | 4.96 | 0.96 | 0.82 | 26.26 | 1.46 | 0.14 | 0.14 | 1 |
| 841 | 229666 | H66542 | 0.11 | 0.53 | 4.77 | 2.48 | 0.09 | 25.48 | 0.07 | 0.08 | 0.43 | 1 |
| 842 | 744627 | AA621310 | 0.82 | 3.87 | 4.71 | 0.95 | 1.14 | 22.95 | 1.24 | 1.38 | 0.62 | 1 |
| 843 | 884462 | AA629707 | 0.15 | 0.66 | 4.42 | 0.13 | 1.05 | 0.32 | 2.09 | 22.55 | 0.40 | 1 |
| 844 | 361291 | AA017466 | 1.00 | 4.41 | 4.41 | 0.57 | 1.15 | 1.35 | 20.54 | 0.78 | 2.06 | 1 |
| 845 | 301735 | N90882 | 0.11 | 0.49 | 4.33 | 19.43 | 2.89 | 1.98 | 0.42 | 1.17 | 0.09 | 1 |
| 846 | 753076 | AA436479 | 2.41 | 10.37 | 4.30 | 0.47 | 0.65 | 23.85 | 0.22 | 0.21 | 0.41 | 1 |
| 847 | 510381 | AA055585 | 0.32 | 1.35 | 4.23 | 19.31 | 0.51 | 1.32 | 1.01 | 0.69 | 2.52 | 1 |
| 848 | 448401 | AA777536 | 0.45 | 1.91 | 4.21 | 0.91 | 1.74 | 18.40 | 0.98 | 2.09 | 1.12 | 1 |
| 849 | 504940 | AA150619 | 0.17 | 0.70 | 4.16 | 1.19 | 1.74 | 0.82 | 1.23 | 19.01 | 0.95 | 1 |
| 850 | 122298 | T99158 | 0.42 | 1.71 | 4.08 | 1.32 | 2.06 | 1.18 | 1.28 | 2.94 | 15.68 | 1 |
| 851 | 781442 | AA428603 | 0.39 | 1.56 | 4.03 | 1.32 | 1.09 | 0.51 | 0.36 | 20.03 | 0.88 | 1 |
| 852 | 430004 | AA034014 | 0.23 | 0.92 | 3.97 | 1.74 | 1.54 | 0.57 | 0.04 | 17.96 | 1.99 | 1 |
| 853 | 23421 | R38261 | 0.58 | 2.28 | 3.92 | 2.13 | 2.98 | 1.30 | 1.23 | 14.18 | 1.67 | 1 |
| 854 | 824211 | AA490975 | 0.43 | 1.65 | 3.80 | 1.08 | 1.29 | 15.59 | 1.72 | 2.23 | 0.91 | 1 |
| 855 | 43065 | R61866 | 0.12 | 0.43 | 3.74 | 1.37 | 0.95 | 2.14 | 0.67 | 14.52 | 2.78 | 1 |
| 856 | 347183 | W80666 | 0.46 | 1.65 | 3.58 | 0.59 | 0.93 | 1.22 | 1.92 | 1.22 | 15.62 | 1 |
| 857 | 32381 | R43452 | 0.97 | 3.38 | 3.48 | 2.06 | 1.14 | 1.62 | 10.80 | 2.71 | 2.56 | 1 |
| 858 | 592778 | AA158211 | 0.04 | 0.12 | 3.46 | 0.47 | 0.21 | 1.36 | 0.63 | 1.95 | 16.16 | 1 |
| 859 | 454908 | AA677403 | 0.60 | 2.05 | 3.45 | 0.77 | 0.38 | 1.22 | 14.94 | 1.32 | 2.08 | 1 |
| 860 | 340641 | W56760 | 0.27 | 0.91 | 3.41 | 1.52 | 1.27 | 1.69 | 15.08 | 0.51 | 0.39 | 1 |
| 861 | 897722 | AA598983 | 0.08 | 0.26 | 3.40 | 16.70 | 0.81 | 0.76 | 0.95 | 1.03 | 0.13 | 1 |
| 862 | 866882 | AA679352 | 0.19 | 0.64 | 3.36 | 17.69 | 1.01 | 0.60 | 0.60 | 0.21 | 0.05 | 1 |
| 863 | 130664 | R22579 | 0.66 | 2.22 | 3.35 | 1.17 | 0.97 | 14.33 | 1.21 | 1.19 | 1.22 | 1 |
| 864 | 1606080 | AA995508 | 0.47 | 1.59 | 3.34 | 1.80 | 1.58 | 1.72 | 1.32 | 11.93 | 1.68 | 1 |
| 865 | 768260 | AA424950 | 0.82 | 2.69 | 3.28 | 1.66 | 12.82 | 2.38 | 0.89 | 1.08 | 0.83 | 1 |
| 866 | 48930 | H10028 | 0.28 | 0.92 | 3.27 | 1.15 | 0.99 | 0.74 | 1.86 | 13.18 | 1.89 | 1 |
| 867 | 1035765 | AA629117 | 10.22 | 33.26 | 3.25 | 0.55 | 0.26 | 1.17 | 15.74 | 1.49 | 0.32 | 1 |
| 868 | 591025 | AA159068 | 0.21 | 0.67 | 3.24 | 1.63 | 0.50 | 2.32 | 1.36 | 2.40 | 11.24 | 1 |
| 869 | 194515 | R86242 | 0.58 | 1.87 | 3.24 | 6.47 | 2.02 | 2.78 | 2.94 | 2.78 | 2.43 | 1 |
| 870 | 308412 | N93790 | 0.02 | 0.05 | 3.21 | 1.60 | 0.62 | 1.66 | 0.62 | 12.88 | 1.87 | 1 |
| 871 | 787449 | AA418000 | 2.79 | 8.93 | 3.20 | 1.31 | 1.70 | 0.78 | 2.24 | 10.28 | 2.91 | 1 |
| 872 | 809981 | AA455197 | 0.09 | 0.28 | 3.19 | 0.83 | 0.72 | 0.87 | 0.94 | 0.35 | 15.41 | 1 |
| 873 | 322175 | W37778 | 1.80 | 5.71 | 3.18 | 2.63 | 6.05 | 2.60 | 2.45 | 2.92 | 2.41 | 1 |
| 874 | 287770 | N62251 | 0.10 | 0.31 | 3.14 | 1.79 | 0.93 | 12.98 | 2.84 | 0.10 | 0.23 | 1 |
| 875 | 51216 | H18471 | 0.33 | 1.03 | 3.13 | 1.22 | 1.79 | 1.32 | 1.82 | 11.65 | 1.01 | 1 |
| 876 | 591683 | AA147214 | 0.03 | 0.10 | 3.13 | 0.62 | 0.57 | 11.65 | 2.53 | 1.55 | 1.85 | 1 |
| 877 | 51951 | H24317 | 0.33 | 1.01 | 3.10 | 1.50 | 12.98 | 1.31 | 1.06 | 0.77 | 1.00 | 1 |
| 878 | 429175 | AA004824 | 0.19 | 0.58 | 3.08 | 1.55 | 6.34 | 2.94 | 2.52 | 2.46 | 2.64 | 1 |
| 879 | 34449 | R44164 | 0.46 | 1.41 | 3.05 | 0.95 | 11.69 | 0.77 | 1.45 | 1.05 | 2.37 | 1 |
| 880 | 280217 | N47941 | 6.29 | 19.12 | 3.04 | 1.96 | 1.84 | 0.85 | 0.83 | 11.66 | 1.12 | 1 |
| 881 | 380838 | AA017141 | 0.34 | 1.01 | 3.00 | 2.60 | 1.86 | 2.57 | 1.66 | 1.18 | 8.11 | 1 |
| 882 | 730946 | AA416543 | 1.11 | 3.32 | 2.98 | 0.90 | 0.96 | 12.90 | 1.25 | 1.05 | 0.81 | 1 |
| 883 | 795297 | AA454155 | 2.01 | 5.93 | 2.95 | 0.60 | 0.67 | 13.67 | 0.74 | 1.21 | 0.81 | 1 |
| 884 | 199237 | R95773 | 0.39 | 1.13 | 2.91 | 2.12 | 2.71 | 1.68 | 7.41 | 1.89 | 1.65 | 1 |
| 885 | 462003 | AA780027 | 0.60 | 1.73 | 2.91 | 1.15 | 2.19 | 2.85 | 1.86 | 7.67 | 1.73 | 1 |
| 886 | 703943 | AA279145 | 3.01 | 8.74 | 2.90 | 2.81 | 2.95 | 1.84 | 5.15 | 2.56 | 2.12 | 1 |
| 887 | 781027 | AA446031 | 0.63 | 1.82 | 2.90 | 1.39 | 1.34 | 1.32 | 1.43 | 1.66 | 10.26 | 1 |
| 888 | 625516 | AA187595 | 0.02 | 0.06 | 2.89 | 0.52 | 1.94 | 0.52 | 2.68 | 2.19 | 9.52 | 1 |
| 889 | 838478 | AA457517 | 4.06 | 11.68 | 2.88 | 1.28 | 0.45 | 1.22 | 10.05 | 1.44 | 2.83 | 1 |
| 890 | 46367 | H09959 | 1.41 | 4.06 | 2.88 | 2.57 | 7.47 | 2.29 | 1.27 | 1.75 | 1.91 | 1 |
| 891 | 213753 | H72322 | 0.26 | 0.73 | 2.87 | 1.57 | 2.40 | 2.53 | 1.74 | 7.18 | 1.79 | 1 |
| 892 | 126449 | R06706 | 0.22 | 0.63 | 2.87 | 0.97 | 2.25 | 1.77 | 1.73 | 9.16 | 1.31 | 1 |
| 893 | 305243 | N95007 | 0.60 | 1.70 | 2.86 | 1.99 | 2.55 | 2.98 | 1.18 | 2.07 | 6.39 | 1 |
| 894 | 254117 | N22230 | 1.22 | 3.49 | 2.86 | 0.86 | 0.81 | 12.24 | 1.03 | 1.03 | 1.16 | 1 |
| 895 | 199285 | R95893 | 0.14 | 0.41 | 2.85 | 2.05 | 2.36 | 1.80 | 2.15 | 7.24 | 1.48 | 1 |
| 896 | 51916 | H22563 | 0.74 | 2.10 | 2.84 | 2.50 | 2.20 | 2.20 | 2.52 | 5.64 | 1.98 | 1 |
| 897 | 111370 | T85173 | 1.89 | 5.34 | 2.83 | 1.63 | 1.87 | 1.92 | 1.04 | 2.87 | 7.65 | 1 |
| 898 | 376435 | AA039713 | 1.18 | 3.34 | 2.83 | 1.76 | 2.11 | 0.92 | 7.08 | 2.69 | 2.39 | 1 |
| 899 | 24676 | R38809 | 0.40 | 1.13 | 2.81 | 1.10 | 2.13 | 1.85 | 1.95 | 7.78 | 2.07 | 1 |
| 900 | 127931 | R08938 | 0.66 | 1.85 | 2.79 | 1.21 | 0.97 | 10.42 | 1.72 | 1.24 | 1.21 | 1 |
| 901 | 77915 | T61323 | 8.10 | 22.59 | 2.79 | 1.00 | 0.49 | 0.62 | 2.18 | 0.57 | 11.87 | 1 |
| 902 | 327461 | W20438 | 0.76 | 2.12 | 2.78 | 2.05 | 2.09 | 2.59 | 5.69 | 2.57 | 1.69 | 1 |
| 903 | 40263 | R55261 | 0.15 | 0.41 | 2.78 | 1.94 | 8.29 | 1.45 | 1.03 | 2.33 | 1.61 | 1 |
| 904 | 826109 | AA521327 | 0.40 | 1.11 | 2.78 | 1.65 | 1.97 | 2.83 | 1.66 | 2.95 | 5.61 | 1 |
| 905 | 113160 | T83842 | 0.66 | 1.83 | 2.77 | 1.55 | 1.53 | 9.91 | 1.30 | 1.08 | 1.26 | 1 |
| 906 | 288768 | N62498 | 0.35 | 0.96 | 2.77 | 1.16 | 1.50 | 1.73 | 1.69 | 10.48 | 0.03 | 1 |
| 907 | 289936 | N59336 | 0.57 | 1.58 | 2.76 | 1.73 | 2.11 | 1.80 | 8.37 | 1.28 | 1.29 | 1 |
| 908 | 770868 | AA434487 | 0.09 | 0.26 | 2.75 | 12.49 | 0.46 | 0.72 | 1.13 | 0.69 | 1.02 | 1 |
| 909 | 197779 | R93515 | 1.43 | 3.91 | 2.74 | 0.94 | 1.29 | 10.19 | 1.11 | 1.44 | 1.46 | 1 |
| 910 | 140831 | R67218 | 0.94 | 2.57 | 2.73 | 1.07 | 1.33 | 9.71 | 1.37 | 1.26 | 1.62 | 1 |
| 911 | 51749 | H24329 | 2.85 | 7.75 | 2.72 | 1.73 | 1.78 | 2.66 | 6.30 | 1.99 | 1.87 | 1 |
| 912 | 280977 | N50864 | 1.00 | 2.71 | 2.71 | 1.62 | 1.49 | 1.30 | 10.18 | 0.79 | 0.90 | 1 |
| 913 | 41835 | R54212 | 0.33 | 0.90 | 2.70 | 8.20 | 1.63 | 1.75 | 1.84 | 1.63 | 1.16 | 1 |
| 914 | 951125 | AA620556 | 15.08 | 40.69 | 2.70 | 2.31 | 2.72 | 2.53 | 6.58 | 1.45 | 0.60 | 1 |
| 915 | 51011 | H19242 | 4.02 | 10.78 | 2.68 | 1.31 | 1.50 | 2.02 | 7.45 | 2.53 | 1.28 | 1 |
| 916 | 44303 | H06380 | 1.99 | 5.32 | 2.68 | 1.57 | 2.81 | 1.64 | 1.80 | 5.50 | 2.76 | 1 |
| 917 | 296838 | W01211 | 0.61 | 1.63 | 2.68 | 1.80 | 6.53 | 2.29 | 1.39 | 1.88 | 2.19 | 1 |
| 918 | 139641 | R64231 | 0.12 | 0.31 | 2.67 | 1.98 | 7.39 | 1.13 | 2.16 | 1.48 | 1.91 | 1 |
| 919 | 609228 | AA179161 | 0.09 | 0.25 | 2.87 | 0.51 | 2.46 | 0.93 | 1.38 | 0.33 | 10.39 | 1 |
| 920 | 743452 | AA609368 | 0.58 | 1.54 | 2.64 | 1.47 | 1.50 | 8.81 | 1.78 | 1.27 | 1.02 | 1 |
| 921 | 289706 | N62948 | 0.33 | 0.87 | 2.63 | 2.06 | 6.78 | 1.41 | 2.87 | 1.46 | 1.20 | 1 |
| 922 | 505538 | AA147594 | 0.09 | 0.25 | 2.63 | 2.25 | 0.11 | 2.32 | 0.83 | 1.71 | 8.55 | 1 |
| 923 | 53022 | R15880 | 0.89 | 2.34 | 2.62 | 2.59 | 1.25 | 1.32 | 6.01 | 1.69 | 2.86 | 1 |
| 924 | 273546 | N33274 | 2.51 | 6.55 | 2.61 | 2.25 | 2.58 | 2.24 | 6.64 | 0.98 | 1.01 | 1 |
| 925 | 843049 | AA485983 | 0.10 | 0.25 | 2.61 | 2.59 | 1.11 | 8.50 | 1.27 | 0.72 | 1.45 | 1 |
| 926 | 594517 | AA169645 | 31.25 | 81.32 | 2.60 | 1.36 | 2.19 | 2.32 | 6.51 | 1.76 | 1.46 | 1 |
| 927 | 453175 | AA700218 | 0.99 | 2.58 | 2.60 | 5.15 | 2.24 | 1.63 | 2.78 | 1.77 | 2.03 | 1 |
| 928 | 360357 | AA013355 | 0.26 | 0.86 | 2.60 | 1.94 | 9.92 | 1.20 | 1.46 | 0.37 | 0.70 | 1 |
| 929 | 345525 | W72437 | 3.95 | 10.21 | 2.58 | 2.40 | 2.96 | 1.23 | 5.65 | 1.74 | 1.53 | 1 |
| 930 | 344274 | W73811 | 0.13 | 0.33 | 2.58 | 0.08 | 0.69 | 12.77 | 0.53 | 0.28 | 1.15 | 1 |
| 931 | 399152 | AA733105 | 0.03 | 0.08 | 2.58 | 9.39 | 0.82 | 1.72 | 2.27 | 0.98 | 0.31 | 1 |
| 932 | 767345 | AA418564 | 1.03 | 2.65 | 2.58 | 1.48 | 1.68 | 2.03 | 2.47 | 5.56 | 2.26 | 1 |
| 933 | 743727 | AA629355 | 0.86 | 2.22 | 2.58 | 0.98 | 2.46 | 5.63 | 1.51 | 1.91 | 2.98 | 1 |
| 934 | 810625 | AA464755 | 0.07 | 0.18 | 2.57 | 1.64 | 1.73 | 1.89 | 1.42 | 7.19 | 1.57 | 1 |
| 935 | 1476181 | AA872034 | 0.20 | 0.51 | 2.57 | 1.98 | 1.32 | 1.27 | 7.42 | 2.20 | 1.24 | 1 |
| 936 | 814154 | AA496252 | 0.87 | 2.23 | 2.57 | 1.20 | 1.20 | 1.67 | 8.80 | 0.59 | 1.96 | 1 |
| 937 | 34832 | R45157 | 0.59 | 1.53 | 2.57 | 1.13 | 1.11 | 1.07 | 0.94 | 0.72 | 10.42 | 1 |
| 938 | 435820 | AA701519 | 0.50 | 1.29 | 2.56 | 2.29 | 1.57 | 2.64 | 1.01 | 0.54 | 7.34 | 1 |
| 939 | 130703 | R22003 | 3.70 | 9.47 | 2.56 | 1.96 | 5.20 | 1.35 | 2.96 | 1.57 | 2.33 | 1 |
| 940 | 30428 | R42061 | 3.15 | 8.00 | 2.54 | 1.75 | 2.23 | 0.74 | 2.28 | 2.59 | 5.66 | 1 |
| 941 | 299162 | N75473 | 0.03 | 0.07 | 2.54 | 0.36 | 0.36 | 2.96 | 9.96 | 0.36 | 1.25 | 1 |
| 942 | 267679 | N25552 | 0.58 | 1.47 | 2.53 | 2.42 | 5.39 | 1.41 | 2.09 | 1.48 | 2.38 | 1 |
| 943 | 145383 | R77919 | 0.10 | 0.24 | 2.52 | 1.76 | 1.91 | 1.19 | 1.86 | 8.26 | 0.17 | 1 |
| 944 | 154720 | R55220 | 0.05 | 0.13 | 2.52 | 2.29 | 7.29 | 1.91 | 0.19 | 1.86 | 1.60 | 1 |
| 945 | 395955 | AA757584 | 0.49 | 1.22 | 2.52 | 1.20 | 1.50 | 1.33 | 0.70 | 9.11 | 1.24 | 1 |
| 946 | 412949 | AA707840 | 0.92 | 2.31 | 2.51 | 1.63 | 5.07 | 2.31 | 1.74 | 1.69 | 2.63 | 1 |
| 947 | 120892 | T95898 | 3.38 | 8.48 | 2.51 | 1.12 | 1.30 | 2.13 | 1.84 | 1.86 | 6.82 | 1 |
| 948 | 271989 | N31935 | 0.18 | 0.45 | 2.50 | 1.45 | 2.91 | 1.52 | 1.12 | 5.41 | 2.57 | 1 |
| 949 | 592592 | AA159577 | 3.36 | 8.37 | 2.49 | 1.59 | 0.54 | 2.12 | 9.45 | 0.77 | 0.49 | 1 |
| 950 | 591055 | AA161097 | 0.08 | 0.20 | 2.49 | 7.00 | 1.24 | 2.42 | 1.57 | 1.07 | 1.62 | 1 |
| 951 | 267252 | N24579 | 0.83 | 2.07 | 2.48 | 6.02 | 2.69 | 1.38 | 1.52 | 1.97 | 1.31 | 1 |
| 952 | 53393 | R16245 | 1.11 | 2.75 | 2.48 | 1.34 | 1.22 | 0.91 | 1.08 | 1.21 | 9.12 | 1 |
| 953 | 327058 | AA284267 | 0.16 | 0.39 | 2.47 | 9.38 | 1.43 | 0.13 | 1.53 | 1.88 | 0.48 | 1 |
| 954 | 294544 | N69425 | 0.51 | 1.25 | 2.47 | 2.70 | 5.07 | 2.03 | 2.61 | 0.94 | 1.48 | 1 |
| 955 | 202904 | H54020 | 28.65 | 70.79 | 2.47 | 2.13 | 1.92 | 1.40 | 5.51 | 1.96 | 1.92 | 1 |
| 956 | 112456 | T90971 | 0.27 | 0.67 | 2.47 | 1.93 | 2.00 | 1.57 | 1.15 | 5.60 | 2.54 | 1 |
| 957 | 810779 | AA481754 | 0.25 | 0.61 | 2.47 | 1.81 | 1.94 | 1.23 | 1.48 | 6.78 | 1.56 | 1 |
| 958 | 431933 | AA678164 | 1.11 | 2.73 | 2.46 | 1.61 | 5.16 | 2.27 | 1.48 | 2.27 | 1.99 | 1 |
| 959 | 359653 | AA010872 | 0.32 | 0.80 | 2.46 | 0.87 | 2.24 | 2.13 | 1.46 | 1.27 | 6.81 | 1 |
| 960 | 681885 | AA258157 | 0.61 | 1.51 | 2.46 | 1.43 | 2.61 | 1.59 | 5.29 | 1.80 | 2.03 | 1 |
| 961 | 239199 | H70942 | 0.91 | 2.25 | 2.46 | 0.90 | 0.75 | 10.09 | 1.08 | 0.89 | 1.07 | 1 |
| 962 | 66564 | T67058 | 0.31 | 0.76 | 2.46 | 2.60 | 7.66 | 0.56 | 0.87 | 1.05 | 1.99 | 1 |
| 963 | 215000 | H73241 | 3.61 | 8.84 | 2.45 | 2.77 | 1.25 | 0.82 | 1.18 | 2.92 | 5.77 | 1 |
| 964 | 810773 | AA481751 | 0.87 | 2.12 | 2.44 | 1.12 | 1.19 | 8.67 | 1.17 | 1.26 | 1.22 | 1 |
| 965 | 282000 | N51107 | 0.53 | 1.29 | 2.44 | 2.00 | 1.90 | 5.73 | 1.70 | 1.87 | 1.43 | 1 |
| 966 | 201301 | R99573 | 2.54 | 6.16 | 2.42 | 1.35 | 2.68 | 2.98 | 5.20 | 1.37 | 0.96 | 1 |
| 967 | 73170 | AA428514 | 0.17 | 0.40 | 2.42 | 1.55 | 1.08 | 1.04 | 0.90 | 9.27 | 0.68 | 1 |
| 968 | 897656 | AA496796 | 5.26 | 12.74 | 2.42 | 2.58 | 6.50 | 0.47 | 1.05 | 1.75 | 2.16 | 1 |
| 969 | 111884 | T84996 | 0.03 | 0.07 | 2.41 | 1.89 | 1.77 | 2.00 | 1.45 | 2.36 | 5.02 | 1 |
| 970 | 744897 | AA625784 | 1.69 | 4.06 | 2.40 | 1.57 | 1.49 | 2.43 | 5.19 | 2.25 | 1.49 | 1 |
| 971 | 46411 | H09164 | 3.30 | 7.91 | 2.40 | 1.72 | 1.78 | 2.67 | 5.14 | 1.19 | 1.88 | 1 |
| 972 | 796761 | AAA60729 | 0.59 | 1.42 | 2.40 | 1.08 | 1.51 | 5.80 | 2.12 | 2.35 | 1.52 | 1 |
| 973 | 1031273 | AA609054 | 0.38 | 0.91 | 2.39 | 1.15 | 7.94 | 1.69 | 0.99 | 0.90 | 1.70 | 1 |
| 974 | 46129 | H09529 | 0.07 | 0.16 | 2.39 | 0.81 | 1.22 | 1.96 | 0.82 | 6.90 | 2.65 | 1 |
| 975 | 220369 | H86816 | 1.11 | 2.65 | 2.39 | 1.00 | 1.09 | 1.16 | 1.46 | 7.40 | 2.27 | 1 |
| 976 | 1292170 | AA705819 | 1.46 | 3.50 | 2.39 | 1.36 | 1.60 | 8.89 | 1.05 | 0.54 | 0.88 | 1 |
| 977 | 356711 | W84560 | 0.78 | 1.86 | 2.38 | 1.32 | 2.39 | 1.02 | 1.38 | 6.51 | 1.88 | 1 |
| 978 | 430884 | AA678203 | 0.65 | 1.56 | 2.38 | 1.54 | 2.21 | 2.38 | 5.04 | 1.26 | 1.64 | 1 |
| 979 | 35575 | R45964 | 2.28 | 5.43 | 2.38 | 1.71 | 0.91 | 2.32 | 2.25 | 1.78 | 5.28 | 1 |
| 980 | 84079 | T71008 | 0.53 | 1.26 | 2.37 | 0.80 | 1.53 | 1.02 | 8.43 | 1.41 | 1.04 | 1 |
| 981 | 1388395 | AA844141 | 0.30 | 0.72 | 2.37 | 0.89 | 0.90 | 0.69 | 0.41 | 10.29 | 1.05 | 1 |
| 982 | 197793 | R93729 | 0.76 | 1.81 | 2.37 | 1.39 | 1.02 | 8.38 | 2.45 | 0.70 | 0.28 | 1 |
| 983 | 26738 | R39891 | 7.65 | 18.11 | 2.37 | 0.64 | 1.43 | 1.12 | 6.66 | 2.83 | 1.71 | 1 |
| 984 | 39899 | R52824 | 0.60 | 1.41 | 2.36 | 1.28 | 0.96 | 1.27 | 8.99 | 1.09 | 0.62 | 1 |
| 985 | 278815 | N34924 | 0.13 | 0.32 | 2.36 | 7.54 | 0.57 | 2.63 | 1.43 | 1.70 | 0.30 | 1 |
| 986 | 430007 | AA034115 | 0.48 | 1.14 | 2.35 | 6.87 | 2.92 | 0.89 | 0.84 | 0.87 | 1.75 | 1 |
| 987 | 121615 | T97592 | 0.44 | 1.04 | 2.35 | 7.31 | 1.59 | 1.65 | 1.23 | 1.13 | 1.21 | 1 |
| 988 | 826303 | AA521016 | 0.54 | 1.28 | 2.35 | 1.29 | 1.46 | 1.13 | 1.43 | 7.46 | 1.30 | 1 |
| 989 | 34442 | R44985 | 1.70 | 4.00 | 2.35 | 0.88 | 0.88 | 2.43 | 8.64 | 0.85 | 0.41 | 1 |
| 990 | 742542 | AA400010 | 0.20 | 0.46 | 2.34 | 1.71 | 2.12 | 1.52 | 1.14 | 5.44 | 2.13 | 1 |
| 991 | 327678 | W23758 | 0.30 | 0.70 | 2.34 | 1.23 | 1.45 | 1.58 | 5.33 | 1.78 | 2.68 | 1 |
| 992 | 160656 | H24954 | 0.07 | 0.17 | 2.34 | 0.52 | 0.74 | 0.97 | 1.18 | 1.22 | 9.40 | 1 |
| 993 | 285114 | N20820 | 0.34 | 0.80 | 2.34 | 7.86 | 0.89 | 1.68 | 1.01 | 0.44 | 2.14 | 1 |
| 994 | 626842 | AA191424 | 1.69 | 3.95 | 2.34 | 1.72 | 1.97 | 2.52 | 0.59 | 0.96 | 6.26 | 1 |
| 995 | 291403 | N67762 | 0.04 | 0.10 | 2.33 | 5.38 | 0.72 | 2.23 | 1.44 | 2.12 | 2.12 | 1 |
| 996 | 240318 | H89795 | 0.43 | 1.00 | 2.33 | 1.83 | 2.67 | 1.64 | 5.64 | 1.01 | 1.19 | 1 |
| 997 | 252684 | H88334 | 1.64 | 3.82 | 2.33 | 0.77 | 0.58 | 9.21 | 0.93 | 0.99 | 1.48 | 1 |
| 998 | 302190 | W16724 | 0.18 | 0.41 | 2.33 | 1.08 | 1.73 | 2.18 | 1.05 | 6.70 | 1.23 | 1 |
| 999 | 130742 | R22024 | 0.83 | 1.94 | 2.33 | 1.55 | 1.60 | 7.25 | 1.31 | 1.29 | 0.96 | 1 |
| 1000 | 884083 | AA829911 | 1.31 | 3.05 | 2.33 | 0.67 | 0.64 | 0.61 | 9.75 | 1.41 | 0.87 | 1 |
| 1001 | 427693 | AA002168 | 0.28 | 0.65 | 2.32 | 1.41 | 1.54 | 1.91 | 1.41 | 1.54 | 6.13 | 1 |
| 1002 | 261241 | H98244 | 0.65 | 1.97 | 2.32 | 2.51 | 5.63 | 1.48 | 1.28 | 1.99 | 1.05 | 1 |
| 1003 | 431029 | AA758470 | 0.06 | 0.13 | 2.32 | 5.12 | 1.92 | 2.46 | 2.71 | 0.86 | 0.85 | 1 |
| 1004 | 381996 | AA001536 | 0.46 | 1.07 | 2.32 | 1.41 | 0.59 | 0.79 | 9.46 | 0.33 | 1.34 | 1 |
| 1005 | 629498 | AA192765 | 2.16 | 4.99 | 2.32 | 0.93 | 1.17 | 8.26 | 1.19 | 1.30 | 1.05 | 1 |
| 1006 | 32444 | R42831 | 0.03 | 0.06 | 2.30 | 1.84 | 0.79 | 1.98 | 2.04 | 2.01 | 5.13 | 1 |
| 1007 | 428778 | AA005228 | 0.25 | 0.56 | 2.29 | 0.96 | 0.54 | 0.17 | 2.06 | 9.31 | 0.70 | 1 |
| 1008 | 1030738 | AA608952 | 0.54 | 1.23 | 2.29 | 1.63 | 1.43 | 7.27 | 1.22 | 1.36 | 0.83 | 1 |
| 1009 | 773500 | AA427927 | 18.96 | 43.36 | 2.29 | 0.95 | 1.38 | 1.79 | 6.04 | 2.92 | 0.65 | 1 |
| 1010 | 305809 | N90051 | 0.92 | 2.10 | 2.28 | 0.83 | 1.72 | 1.21 | 5.27 | 2.14 | 2.50 | 1 |
| 1011 | 30502 | R41605 | 0.11 | 0.26 | 2.28 | 5.01 | 2.64 | 2.00 | 1.62 | 1.22 | 1.16 | 1 |
| 1012 | 897791 | AA598520 | 0.68 | 1.55 | 2.27 | 0.80 | 0.89 | 1.89 | 1.85 | 6.37 | 1.85 | 1 |
| 1013 | 430052 | AA034179 | 0.40 | 0.92 | 2.27 | 0.70 | 1.27 | 1.75 | 2.26 | 5.09 | 2.57 | 1 |
| 1014 | 46238 | H10665 | 0.17 | 0.38 | 2.27 | 5.57 | 1.44 | 2.71 | 1.32 | 0.42 | 2.18 | 1 |
| 1015 | 703739 | AA278749 | 8.47 | 19.24 | 2.27 | 0.85 | 0.71 | 0.71 | 1.69 | 0.62 | 9.05 | 1 |
| 1016 | 342720 | W68396 | 0.42 | 0.98 | 2.27 | 0.42 | 0.02 | 0.87 | 1.58 | 9.54 | 1.19 | 1 |
| 1017 | 25156 | R38935 | 1.15 | 2.61 | 2.27 | 1.08 | 5.81 | 2.33 | 2.50 | 0.73 | 1.15 | 1 |
| 1018 | 754854 | AA411607 | 0.08 | 0.18 | 2.26 | 1.84 | 2.38 | 0.81 | 1.73 | 0.47 | 8.54 | 1 |
| 1019 | 377671 | AA055979 | 0.08 | 0.17 | 2.25 | 2.09 | 0.54 | 1.89 | 0.13 | 8.30 | 2.58 | 1 |
| 1020 | 271799 | N31605 | 2.29 | 5.10 | 2.25 | 0.33 | 0.35 | 0.39 | 11.15 | 1.12 | 0.17 | 1 |
| 1021 | 448885 | AA777670 | 5.05 | 11.38 | 2.25 | 0.83 | 1.65 | 0.93 | 6.21 | 2.11 | 1.75 | 1 |
| 1022 | 171936 | H19068 | 0.37 | 0.84 | 2.24 | 1.38 | 0.94 | 2.83 | 5.99 | 1.42 | 0.89 | 1 |
| 1023 | 379709 | AA776089 | 1.21 | 2.70 | 2.23 | 0.48 | 1.43 | 0.94 | 1.26 | 8.84 | 0.44 | 1 |
| 1024 | 325012 | AA284283 | 0.09 | 0.21 | 2.23 | 0.18 | 1.29 | 8.10 | 2.62 | 1.52 | 1.85 | 1 |
| 1025 | 363058 | AA019316 | 0.22 | 0.48 | 2.22 | 7.67 | 0.92 | 1.02 | 1.27 | 1.25 | 1.20 | 1 |
| 1026 | 48914 | H10208 | 0.78 | 1.73 | 2.22 | 6.51 | 1.41 | 1.00 | 1.35 | 1.81 | 1.21 | 1 |
| 1027 | 23819 | R39440 | 0.38 | 0.85 | 2.21 | 0.63 | 1.56 | 1.02 | 8.04 | 1.07 | 2.97 | 1 |
| 1028 | 772960 | AA476257 | 0.54 | 1.18 | 2.21 | 1.60 | 2.11 | 5.18 | 1.73 | 1.36 | 1.27 | 1 |
| 1029 | 71730 | T51229 | 0.09 | 0.20 | 2.21 | 0.08 | 0.07 | 0.70 | 1.05 | 10.80 | 0.55 | 1 |
| 1030 | 261571 | H98758 | 0.03 | 0.07 | 2.20 | 1.59 | 5.46 | 0.58 | 1.80 | 1.64 | 2.18 | 1 |
| 1031 | 814053 | AA486495 | 10.76 | 23.71 | 2.20 | 0.66 | 0.35 | 2.52 | 9.15 | 0.27 | 0.27 | 1 |
| 1032 | 1292887 | AA776755 | 0.48 | 1.05 | 2.20 | 1.24 | 1.48 | 1.02 | 0.71 | 7.63 | 1.13 | 1 |
| 1033 | 324680 | AA284278 | 0.25 | 0.54 | 2.20 | 1.76 | 1.64 | 5.61 | 1.60 | 0.98 | 1.59 | 1 |
| 1034 | 72000 | T52320 | 4.01 | 8.81 | 2.20 | 0.86 | 1.78 | 1.92 | 5.87 | 1.47 | 1.30 | 1 |
| 1035 | 1467109 | AA883170 | 0.57 | 1.28 | 2.19 | 1.91 | 2.18 | 1.40 | 0.97 | 1.53 | 5.21 | 1 |
| 1036 | 244951 | N54551 | 0.25 | 0.55 | 2.19 | 0.59 | 1.13 | 1.73 | 0.09 | 0.04 | 9.58 | 1 |
| 1037 | 593280 | AA185512 | 1.86 | 4.07 | 2.19 | 0.77 | 0.65 | 9.52 | 0.83 | 0.78 | 0.62 | 1 |
| 1038 | 298040 | N73584 | 1.50 | 3.29 | 2.19 | 1.03 | 1.66 | 1.99 | 1.69 | 1.34 | 5.45 | 1 |
| 1039 | 451871 | AA708935 | 0.16 | 0.35 | 2.18 | 0.69 | 0.71 | 0.81 | 0.50 | 0.22 | 10.37 | 1 |
| 1040 | 795588 | AA459892 | 0.78 | 1.70 | 2.18 | 1.13 | 1.28 | 6.35 | 1.41 | 1.27 | 1.67 | 1 |
| 1041 | 168199 | R88248 | 0.05 | 0.11 | 2.18 | 1.50 | 0.53 | 2.15 | 6.01 | 1.14 | 1.76 | 1 |
| 1042 | 46183 | H09818 | 0.08 | 0.18 | 2.18 | 1.76 | 0.47 | 8.54 | 0.12 | 0.12 | 2.07 | 1 |
| 1043 | 344806 | W72838 | 0.42 | 0.91 | 2.18 | 1.28 | 1.47 | 1.24 | 1.70 | 1.05 | 6.35 | 1 |
| 1044 | 51939 | H22958 | 0.42 | 0.91 | 2.18 | 8.97 | 0.79 | 0.96 | 1.07 | 0.55 | 0.73 | 1 |
| 1045 | 280882 | N50806 | 0.05 | 0.11 | 2.17 | 10.72 | 0.90 | 0.18 | 0.19 | 0.86 | 0.19 | 1 |
| 1046 | 1461070 | AA890147 | 0.51 | 1.09 | 2.17 | 1.57 | 1.72 | 1.25 | 0.82 | 6.45 | 1.19 | 1 |
| 1047 | 430235 | AA010223 | 0.04 | 0.09 | 2.17 | 2.12 | 2.37 | 0.25 | 0.25 | 0.25 | 7.77 | 1 |
| 1048 | 212398 | H68312 | 0.70 | 1.52 | 2.16 | 1.47 | 1.12 | 1.37 | 1.17 | 5.78 | 2.04 | 1 |
| 1049 | 147834 | R81831 | 1.73 | 3.73 | 2.16 | 2.42 | 1.28 | 0.74 | 0.85 | 5.42 | 2.24 | 1 |
| 1050 | 416155 | W86075 | 0.23 | 0.49 | 2.16 | 0.42 | 0.39 | 1.55 | 0.49 | 0.57 | 9.51 | 1 |
| 1051 | 139837 | R62289 | 0.64 | 1.37 | 2.15 | 1.94 | 7.04 | 1.24 | 0.68 | 0.70 | 1.32 | 1 |
| 1052 | 131626 | R23735 | 0.10 | 0.22 | 2.15 | 0.63 | 1.32 | 1.95 | 1.79 | 6.30 | 0.92 | 1 |
| 1053 | 32663 | R43319 | 0.50 | 1.07 | 2.15 | 0.99 | 1.42 | 1.26 | 7.30 | 1.06 | 0.88 | 1 |
| 1054 | 1031717 | AA609584 | 1.03 | 2.22 | 2.15 | 1.83 | 1.79 | 1.80 | 5.23 | 1.14 | 1.10 | 1 |
| 1055 | 254328 | N22272 | 1.39 | 2.97 | 2.15 | 0.66 | 0.43 | 9.65 | 0.73 | 0.79 | 0.60 | 1 |
| 1056 | 788605 | AA452897 | 0.66 | 1.42 | 2.14 | 1.48 | 2.24 | 2.10 | 5.53 | 0.52 | 1.00 | 1 |
| 1057 | 166245 | R88267 | 0.03 | 0.07 | 2.14 | 0.31 | 5.65 | 0.31 | 2.51 | 1.42 | 2.64 | 1 |
| 1058 | 32991 | R44770 | 0.26 | 0.55 | 2.14 | 10.49 | 0.04 | 1.10 | 0.61 | 0.20 | 0.38 | 1 |
| 1059 | 712840 | AA282023 | 0.13 | 0.28 | 2.13 | 1.31 | 0.35 | 1.44 | 0.49 | 8.11 | 1.08 | 1 |
| 1060 | 359153 | AA010136 | 0.47 | 1.00 | 2.13 | 1.35 | 1.22 | 6.15 | 1.77 | 1.37 | 0.92 | 1 |
| 1061 | 207864 | H60581 | 0.42 | 0.89 | 2.13 | 1.08 | 1.48 | 2.05 | 1.23 | 1.34 | 5.57 | 1 |
| 1062 | 395711 | AA757764 | 0.10 | 0.22 | 2.12 | 1.66 | 5.61 | 1.05 | 1.66 | 1.36 | 1.40 | 1 |
| 1063 | 78946 | T61792 | 3.51 | 7.43 | 2.12 | 1.23 | 0.53 | 1.70 | 1.68 | 1.15 | 6.41 | 1 |
| 1064 | 42373 | R67147 | 2.61 | 5.52 | 2.11 | 2.11 | 0.91 | 1.90 | 1.25 | 1.33 | 5.19 | 1 |
| 1065 | 199628 | R96598 | 0.18 | 0.37 | 2.10 | 1.07 | 1.40 | 0.88 | 1.69 | 0.70 | 6.89 | 1 |
| 1066 | 29828 | R42331 | 1.30 | 2.72 | 2.10 | 2.03 | 0.72 | 0.48 | 2.13 | 1.94 | 5.32 | 1 |
| 1067 | 809628 | AA455521 | 0.66 | 1.39 | 2.10 | 6.79 | 1.73 | 0.53 | 1.28 | 0.99 | 1.30 | 1 |
| 1068 | 290561 | N62376 | 2.80 | 5.88 | 2.10 | 1.31 | 1.57 | 0.58 | 1.09 | 0.95 | 7.10 | 1 |
| 1069 | 781088 | AA430002 | 0.62 | 1.31 | 2.10 | 1.23 | 0.99 | 6.61 | 1.56 | 1.28 | 0.92 | 1 |
| 1070 | 282384 | N51987 | 0.51 | 1.06 | 2.09 | 1.05 | 0.99 | 1.90 | 1.23 | 1.17 | 6.21 | 1 |
| 1071 | 762673 | AA447585 | 0.21 | 0.44 | 2.08 | 0.47 | 1.67 | 0.47 | 1.20 | 7.98 | 0.69 | 1 |
| 1072 | 73785 | T54672 | 1.35 | 2.79 | 2.07 | 1.21 | 1.21 | 1.49 | 5.44 | 1.88 | 1.22 | 1 |
| 1073 | 730124 | AA412470 | 0.22 | 0.44 | 2.05 | 2.93 | 1.29 | 0.05 | 2.06 | 5.24 | 0.73 | 1 |
| 1074 | 51493 | H18924 | 0.28 | 0.57 | 2.05 | 1.36 | 0.99 | 0.32 | 1.09 | 7.73 | 0.79 | 1 |
| 1075 | 38497 | R49124 | 0.34 | 0.70 | 2.05 | 0.64 | 1.05 | 0.28 | 0.36 | 9.67 | 0.30 | 1 |
| 1076 | 1048723 | AA620642 | 0.71 | 1.44 | 2.03 | 1.67 | 1.45 | 1.18 | 1.05 | 6.22 | 0.63 | 1 |
| 1077 | 141815 | R70885 | 8.27 | 16.77 | 2.03 | 0.45 | 0.67 | 0.71 | 0.56 | 8.86 | 0.91 | 1 |
| 1078 | 757337 | AA437099 | 1.28 | 2.60 | 2.03 | 0.60 | 0.98 | 0.58 | 2.60 | 1.86 | 5.54 | 1 |
| 1079 | 255659 | N27841 | 0.05 | 0.09 | 2.02 | 7.29 | 1.20 | 0.95 | 0.79 | 1.68 | 0.22 | 1 |
| 1080 | 428169 | AA001876 | 0.20 | 0.41 | 2.02 | 0.05 | 0.31 | 0.75 | 0.65 | 9.69 | 0.69 | 1 |
| 1081 | 950470 | AAA99102 | 2.48 | 5.00 | 2.02 | 1.00 | 1.18 | 6.48 | 1.57 | 0.95 | 0.92 | 1 |
| 1082 | 824044 | AA491206 | 0.09 | 0.18 | 2.01 | 0.11 | 1.13 | 0.92 | 1.59 | 6.92 | 1.39 | 1 |
| 1083 | 1456721 | AA864875 | 12.97 | 26.09 | 2.01 | 1.29 | 1.62 | 1.39 | 5.81 | 1.46 | 0.49 | 1 |
| 1084 | 757156 | AA443938 | 0.49 | 0.98 | 2.01 | 0.95 | 1.21 | 0.63 | 0.37 | 8.19 | 0.71 | 1 |
| 1085 | 252830 | H88261 | 1.63 | 3.26 | 2.00 | 1.12 | 0.93 | 7.24 | 0.89 | 0.87 | 0.98 | 1 |
| 1086 | 24918 | R39069 | 2.08 | 4.16 | 2.00 | 5.45 | 1.50 | 2.33 | 0.65 | 0.59 | 1.50 | 1 |
| 1087 | 1637296 | A1005519 | 25.34 | 50.73 | 2.00 | 1.13 | 1.00 | 1.62 | 5.66 | 1.33 | 1.27 | 1 |
| 1088 | 123539 | R00809 | 0.73 | 1.46 | 2.00 | 1.18 | 1.05 | 6.39 | 1.19 | 1.19 | 0.99 | 1 |
| 1089 | 1461664 | AA885311 | 2.34 | 4.66 | 1.99 | 1.88 | 1.60 | 0.77 | 1.57 | 5.84 | 0.29 | 1 |
| 1090 | 1467207 | AA884713 | 5.60 | 11.14 | 1.99 | 1.05 | 1.01 | 1.86 | 6.15 | 1.30 | 0.58 | 1 |
| 1091 | 79022 | T62179 | 7.99 | 15.78 | 1.98 | 2.00 | 0.48 | 0.69 | 1.76 | 0.57 | 6.35 | 1 |
| 1092 | 37665 | R61374 | 0.03 | 0.06 | 1.96 | 1.33 | 0.87 | 1.28 | 1.57 | 0.30 | 6.37 | 1 |
| 1093 | 452545 | AA778839 | 0.57 | 1.12 | 1.96 | 1.27 | 1.70 | 1.67 | 5.10 | 0.52 | 1.47 | 1 |
| 1094 | 773568 | AA428196 | 0.12 | 0.23 | 1.95 | 0.56 | 2.83 | 0.27 | 0.83 | 7.14 | 0.09 | 1 |
| 1095 | 490994 | AA136699 | 0.35 | 0.69 | 1.95 | 0.70 | 0.61 | 0.97 | 1.65 | 7.29 | 0.49 | 1 |
| 1096 | 41074 | R56604 | 0.21 | 0.40 | 1.95 | 0.77 | 1.40 | 0.53 | 5.73 | 0.84 | 2.42 | 1 |
| 1097 | 257342 | N29638 | 0.51 | 0.99 | 1.95 | 1.38 | 1.30 | 5.48 | 1.16 | 1.50 | 0.86 | 1 |
| 1098 | 897595 | AA496887 | 0.85 | 1.66 | 1.94 | 1.40 | 0.89 | 1.10 | 0.89 | 1.63 | 5.75 | 1 |
| 1099 | 108378 | T77729 | 0.02 | 0.03 | 1.94 | 2.53 | 0.85 | 0.47 | 0.57 | 0.57 | 6.65 | 1 |
| 1100 | 276911 | N34945 | 0.21 | 0.40 | 1.94 | 0.63 | 0.65 | 0.75 | 0.39 | 8.19 | 1.03 | 1 |
| 1101 | 147075 | R80235 | 0.07 | 0.14 | 1.93 | 0.12 | 5.30 | 0.81 | 2.48 | 2.74 | 0.13 | 1 |
| 1102 | 773331 | AA425436 | 10.48 | 20.23 | 1.93 | 0.68 | 0.73 | 2.31 | 5.88 | 1.27 | 0.71 | 1 |
| 1103 | 241077 | H80325 | 3.25 | 8.25 | 1.93 | 0.86 | 1.38 | 1.48 | 5.67 | 1.46 | 0.69 | 1 |
| 1104 | 824074 | AA491227 | 0.18 | 0.34 | 1.92 | 8.92 | 0.68 | 0.53 | 0.51 | 0.32 | 0.57 | 1 |
| 1105 | 796078 | AA460353 | 0.09 | 0.18 | 1.92 | 1.61 | 0.75 | 1.57 | 1.28 | 0.11 | 6.21 | 1 |
| 1106 | 838616 | AA456968 | 1.21 | 2.31 | 1.91 | 0.59 | 0.58 | 7.48 | 0.87 | 1.12 | 0.85 | 1 |
| 1107 | 45501 | H08397 | 0.12 | 0.22 | 1.91 | 2.63 | 2.45 | 0.05 | 5.68 | 0.18 | 0.48 | 1 |
| 1108 | 415279 | W95050 | 0.51 | 0.97 | 1.90 | 1.12 | 0.89 | 5.04 | 0.98 | 0.97 | 2.40 | 1 |
| 1109 | 40299 | R52085 | 0.10 | 0.19 | 1.89 | 1.50 | 2.20 | 0.90 | 5.58 | 0.36 | 0.83 | 1 |
| 1110 | 33894 | R44530 | 0.26 | 0.48 | 1.89 | 0.99 | 0.03 | 1.22 | 1.69 | 7.10 | 0.33 | 1 |
| 1111 | 471641 | AA034945 | 4.72 | 8.92 | 1.89 | 6.06 | 1.75 | 0.60 | 1.19 | 0.80 | 0.95 | 1 |
| 1112 | 742887 | AA406226 | 0.36 | 0.68 | 1.89 | 1.23 | 1.29 | 1.28 | 1.68 | 5.14 | 0.72 | 1 |
| 1113 | 348523 | W73994 | 1.01 | 1.90 | 1.89 | 2.65 | 5.94 | 1.07 | 0.52 | 0.54 | 0.60 | 1 |
| 1114 | 743229 | AA400329 | 0.36 | 0.67 | 1.88 | 0.82 | 0.76 | 0.49 | 5.87 | 1.57 | 1.79 | 1 |
| 1115 | 866702 | AA679180 | 0.04 | 0.08 | 1.88 | 0.25 | 5.80 | 1.45 | 1.99 | 1.54 | 0.25 | 1 |
| 1116 | 150798 | H02550 | 1.20 | 2.25 | 1.87 | 0.95 | 6.61 | 0.98 | 0.62 | 0.79 | 1.29 | 1 |
| 1117 | 183556 | H44032 | 0.28 | 0.52 | 1.87 | 5.21 | 1.11 | 1.34 | 1.43 | 1.10 | 1.02 | 1 |
| 1118 | 243905 | H33965 | 0.19 | 0.35 | 1.88 | 0.27 | 1.33 | 2.60 | 1.31 | 5.59 | 0.08 | 1 |
| 1119 | 731404 | AA41227 | 0.92 | 1.72 | 1.86 | 1.31 | 0.90 | 5.72 | 1.23 | 1.27 | 0.73 | 1 |
| 1120 | 243321 | H94720 | 1.08 | 2.00 | 1.86 | 0.92 | 0.98 | 1.02 | 5.71 | 1.84 | 0.69 | 1 |
| 1121 | 243603 | N49725 | 0.64 | 1.19 | 1.86 | 1.16 | 1.11 | 1.11 | 1.15 | 1.05 | 5.57 | 1 |
| 1122 | 841610 | AA487457 | 0.08 | 0.11 | 1.86 | 1.70 | 5.30 | 2.13 | 0.17 | 1.67 | 0.17 | 1 |
| 1123 | 80371 | T65765 | 1.21 | 2.24 | 1.85 | 0.50 | 6.17 | 1.41 | 0.72 | 1.21 | 1.09 | 1 |
| 1124 | 731029 | AA421269 | 0.06 | 0.15 | 1.85 | 1.86 | 2.25 | 0.69 | 5.70 | 0.12 | 0.67 | 1 |
| 1125 | 284882 | N66737 | 0.33 | 0.61 | 1.85 | 0.44 | 0.82 | 0.56 | 5.74 | 2.61 | 0.92 | 1 |
| 1126 | 1031009 | AA635186 | 5.84 | 10.78 | 1.85 | 0.60 | 0.46 | 1.52 | 7.50 | 0.73 | 0.26 | 1 |
| 1127 | 1030637 | AA608770 | 0.76 | 1.41 | 1.85 | 1.20 | 1.32 | 5.23 | 1.03 | 1.16 | 1.13 | 1 |
| 1128 | 273168 | N36794 | 0.15 | 0.27 | 1.84 | 1.65 | 0.62 | 0.64 | 2.12 | 5.94 | 0.07 | 1 |
| 1129 | 66317 | T68818 | 8.85 | 16.25 | 1.84 | 6.02 | 2.40 | 0.49 | 0.22 | 0.80 | 1.26 | 1 |
| 1130 | 253931 | N22084 | 1.11 | 2.03 | 1.83 | 0.86 | 0.96 | 8.40 | 1.04 | 0.80 | 0.92 | 1 |
| 1131 | 487086 | AA045300 | 2.58 | 4.69 | 1.82 | 0.88 | 0.91 | 5.53 | 1.44 | 1.09 | 1.05 | 1 |
| 1132 | 253598 | H89376 | 1.38 | 2.50 | 1.81 | 0.86 | 0.87 | 5.81 | 0.92 | 0.75 | 1.68 | 1 |
| 1133 | 1056186 | AA621001 | 0.86 | 1.54 | 1.79 | 0.96 | 1.47 | 5.27 | 1.26 | 1.20 | 0.61 | 1 |
| 1134 | 1606837 | AA996028 | 31.95 | 57.14 | 1.79 | 1.13 | 0.53 | 1.83 | 5.43 | 0.98 | 0.82 | 1 |
| 1135 | 343731 | W69213 | 0.06 | 0.11 | 1.78 | 0.21 | 5.41 | 1.10 | 2.06 | 0.17 | 1.77 | 1 |
| 1136 | 25058 | R38894 | 0.09 | 0.15 | 1.78 | 0.12 | 0.55 | 1.10 | 7.92 | 0.89 | 0.12 | 1 |
| 1137 | 257387 | N30704 | 2.69 | 4.78 | 1.78 | 0.89 | 1.40 | 1.21 | 5.01 | 1.13 | 1.03 | 1 |
| 1138 | 128058 | R09729 | 1.13 | 2.00 | 1.77 | 0.80 | 0.64 | 6.31 | 0.75 | 1.01 | 1.13 | 1 |
| 1139 | 397604 | AA701034 | 0.18 | 0.32 | 1.76 | 0.81 | 0.97 | 0.61 | 0.83 | 6.53 | 0.83 | 1 |
| 1140 | 42271 | R60328 | 0.02 | 0.04 | 1.76 | 2.06 | 0.50 | 0.88 | 0.50 | 0.50 | 6.13 | 1 |
| 1141 | 71674 | T57920 | 0.24 | 0.42 | 1.76 | 0.04 | 7.34 | 1.28 | 0.30 | 0.30 | 1.28 | 1 |
| 1142 | 340949 | W57818 | 0.17 | 0.30 | 1.74 | 0.46 | 0.91 | 5.79 | 2.19 | 1.04 | 0.06 | 1 |
| 1143 | 788256 | AA454098 | 0.10 | 0.17 | 1.73 | 0.19 | 5.20 | 1.99 | 0.10 | 2.82 | 0.10 | 1 |
| 1144 | 142397 | R69934 | 0.66 | 1.13 | 1.72 | 0.90 | 1.00 | 1.06 | 0.97 | 0.50 | 5.93 | 1 |
| 1145 | 66902 | T69477 | 0.89 | 1.53 | 1.72 | 0.49 | 0.39 | 0.57 | 6.34 | 1.28 | 1.26 | 1 |
| 1146 | 26506 | R20640 | 0.47 | 0.80 | 1.72 | 0.96 | 0.75 | 1.23 | 5.01 | 1.68 | 0.71 | 1 |
| 1147 | 489476 | AA055673 | 0.11 | 0.20 | 1.72 | 0.86 | 0.09 | 0.31 | 0.90 | 6.93 | 1.23 | 1 |
| 1148 | 795446 | AA453616 | 0.12 | 0.20 | 1.72 | 1.17 | 2.30 | 5.68 | 0.97 | 0.09 | 0.09 | 1 |
| 1149 | 471498 | AA035347 | 0.08 | 0.14 | 1.71 | 0.44 | 5.02 | 1.21 | 2.24 | 0.85 | 0.51 | 1 |
| 1150 | 745001 | AA626011 | 1.43 | 2.45 | 1.71 | 0.67 | 1.30 | 1.30 | 5.72 | 0.93 | 0.35 | 1 |
| 1151 | 609155 | AA176867 | 2.10 | 3.59 | 1.71 | 0.49 | 1.44 | 0.60 | 0.86 | 6.09 | 0.78 | 1 |
| 1152 | 796649 | AA460274 | 0.01 | 0.02 | 1.70 | 0.72 | 0.54 | 5.00 | 2.50 | 0.72 | 0.72 | 1 |
| 1153 | 383003 | AA018866 | 0.40 | 0.68 | 1.70 | 1.05 | 1.03 | 0.60 | 0.60 | 5.68 | 1.23 | 1 |
| 1154 | 271483 | N31808 | 0.07 | 0.12 | 1.69 | 1.75 | 0.14 | 1.28 | 0.83 | 6.01 | 0.14 | 1 |
| 1155 | 48356 | H09936 | 2.53 | 4.28 | 1.69 | 0.88 | 0.60 | 1.14 | 5.86 | 0.92 | 0.76 | 1 |
| 1156 | 825461 | AA504354 | 1.09 | 1.83 | 1.69 | 1.60 | 0.45 | 0.84 | 1.35 | 0.70 | 5.17 | 1 |
| 1157 | 1291950 | AA707450 | 0.32 | 0.55 | 1.69 | 1.71 | 1.08 | 0.81 | 0.41 | 0.91 | 5.19 | 1 |
| 1158 | 418197 | W90384 | 0.96 | 1.60 | 1.68 | 0.87 | 0.68 | 6.32 | 0.82 | 0.78 | 0.50 | 1 |
| 1159 | 133358 | R26859 | 0.61 | 1.01 | 1.68 | 1.52 | 0.76 | 1.20 | 5.19 | 0.64 | 0.62 | 1 |
| 1160 | 289606 | N26769 | 0.06 | 0.10 | 1.65 | 0.27 | 2.45 | 0.38 | 1.34 | 5.30 | 0.16 | 1 |
| 1161 | 1293119 | AA682233 | 0.46 | 0.76 | 1.65 | 1.60 | 1.37 | 1.02 | 0.23 | 0.66 | 5.01 | 1 |
| 1162 | 768953 | AA424920 | 2.45 | 4.03 | 1.65 | 0.50 | 0.28 | 0.39 | 1.09 | 7.12 | 0.50 | 1 |
| 1163 | 772916 | AA479912 | 0.06 | 0.10 | 1.64 | 0.16 | 1.15 | 0.73 | 0.23 | 0.16 | 7.41 | 1 |
| 1164 | 31869 | R43017 | 0.23 | 0.37 | 1.62 | 2.56 | 0.27 | 0.46 | 0.74 | 5.35 | 0.35 | 1 |
| 1166 | 84141 | T71042 | 0.03 | 0.05 | 1.62 | 2.59 | 5.16 | 0.34 | 0.34 | 0.97 | 0.34 | 1 |
| 1166 | 969636 | AA663592 | 1.87 | 3.03 | 1.62 | 0.64 | 0.38 | 0.53 | 5.01 | 2.20 | 0.97 | 1 |
| 1167 | 810697 | AA457696 | 0.14 | 0.22 | 1.61 | 0.07 | 0.07 | 1.15 | 7.87 | 0.18 | 0.34 | 1 |
| 1168 | 37234 | R35283 | 0.07 | 0.11 | 1.61 | 1.33 | 1.88 | 0.60 | 5.00 | 0.14 | 0.68 | 1 |
| 1169 | 186132 | H39560 | 0.24 | 0.38 | 1.59 | 1.04 | 0.41 | 0.24 | 5.63 | 0.11 | 2.13 | 1 |
| 1170 | 112658 | T91168 | 0.10 | 0.16 | 1.59 | 1.13 | 0.87 | 1.25 | 0.62 | 5.54 | 0.10 | 1 |
| 1171 | 798117 | AA460961 | 0.11 | 0.18 | 1.58 | 0.29 | 1.78 | 0.70 | 0.96 | 5.69 | 0.09 | 1 |
| 1172 | 41321 | R58906 | 0.09 | 0.14 | 1.57 | 1.21 | 1.33 | 0.95 | 0.11 | 0.11 | 5.73 | 1 |
| 1173 | 429446 | AA007619 | 0.30 | 0.46 | 1.57 | 0.26 | 0.65 | 0.32 | 1.31 | 0.75 | 6.14 | 1 |
| 1174 | 882571 | AA676515 | 0.11 | 0.17 | 1.55 | 0.89 | 5.96 | 1.61 | 0.09 | 0.09 | 0.66 | 1 |
| 1175 | 448813 | AA777590 | 1.22 | 1.89 | 1.55 | 1.72 | 0.40 | 0.73 | 0.61 | 0.40 | 5.41 | 1 |
| 1176 | 49358 | H15104 | 0.22 | 0.34 | 1.54 | 0.35 | 1.37 | 0.76 | 6.22 | 0.22 | 0.33 | 1 |
| 1177 | 810891 | AA459519 | 0.36 | 0.55 | 1.52 | 0.66 | 0.36 | 0.46 | 0.86 | 6.51 | 0.31 | 1 |
| 1178 | 594123 | AA169475 | 1.91 | 2.89 | 1.51 | 0.83 | 0.75 | 5.42 | 0.73 | 0.70 | 0.66 | 1 |
| 1179 | 869375 | AA679907 | 0.03 | 0.05 | 1.51 | 0.31 | 7.25 | 0.59 | 0.31 | 0.31 | 0.31 | 1 |
| 1180 | 128248 | R11510 | 0.09 | 0.14 | 1.50 | 6.53 | 0.59 | 0.11 | 0.88 | 0.62 | 0.27 | 1 |
| 1181 | 22895 | R38640 | 0.09 | 0.14 | 1.50 | 1.61 | 0.07 | 0.44 | 0.51 | 5.78 | 0.55 | 1 |
| 1182 | 286404 | N67274 | 1.50 | 2.22 | 1.48 | 0.77 | 0.62 | 5.33 | 0.81 | 0.65 | 0.71 | 1 |
| 1183 | 366915 | AA027147 | 0.16 | 0.23 | 1.44 | 0.60 | 0.06 | 0.73 | 1.34 | 5.61 | 0.31 | 1 |
| 1184 | 691465 | AA160695 | 0.04 | 0.06 | 1.42 | 0.40 | 6.04 | 1.35 | 0.20 | 0.25 | 0.25 | 1 |
| 1185 | 344595 | W73634 | 0.37 | 0.52 | 1.41 | 5.41 | 0.92 | 0.24 | 0.82 | 0.44 | 0.63 | 1 |
| 1186 | 781110 | AA430042 | 0.03 | 0.04 | 1.38 | 0.23 | 0.31 | 0.31 | 0.31 | 0.84 | 6.29 | 1 |
| 1187 | 291690 | N87832 | 0.04 | 0.06 | 1.38 | 0.16 | 0.20 | 0.98 | 0.73 | 0.24 | 5.97 | 1 |
| 1188 | 878469 | AA870390 | 1.05 | 1.44 | 1.37 | 0.74 | 0.52 | 0.53 | 0.42 | 5.54 | 0.49 | 1 |
| 1189 | 1402258 | AA875953 | 0.14 | 0.20 | 1.37 | 6.50 | 0.76 | 0.28 | 0.56 | 0.07 | 0.07 | 1 |
| 1190 | 565493 | AA129677 | 0.08 | 0.11 | 1.35 | 0.60 | 0.35 | 0.56 | 0.75 | 0.11 | 5.71 | 1 |
| 1191 | 488207 | AA046430 | 1.78 | 2.37 | 1.33 | 0.52 | 0.46 | 5.19 | 0.72 | 0.66 | 0.46 | 1 |
| 1192 | 42118 | R60722 | 0.07 | 0.09 | 1.33 | 0.60 | 5.91 | 0.14 | 0.16 | 0.27 | 0.91 | 1 |
| 1193 | 52594 | H29216 | 0.06 | 0.07 | 1.31 | 5.93 | 0.29 | 0.18 | 0.76 | 0.18 | 0.50 | 1 |
| 1194 | 854401 | AA688959 | 1.41 | 1.83 | 1.29 | 0.43 | 0.44 | 0.58 | 0.41 | 5.08 | 0.82 | 1 |
| 1195 | 307337 | N95228 | 2.86 | 3.64 | 1.27 | 0.45 | 0.45 | 0.26 | 5.73 | 0.29 | 0.45 | 1 |
| 1196 | 377296 | AA055491 | 0.17 | 0.22 | 1.25 | 0.29 | 0.55 | 0.30 | 0.43 | 0.47 | 5.44 | 1 |
| 1197 | 592728 | AA160670 | 0.09 | 0.10 | 1.21 | 0.12 | 0.12 | 6.44 | 0.12 | 0.12 | 0.33 | 1 |
| 1198 | 322617 | W39343 | 0.16 | 0.19 | 1.19 | 0.54 | 0.19 | 0.14 | 0.61 | 0.38 | 5.26 | 1 |
| 1199 | 503545 | AA131516 | 0.09 | 0.10 | 1.18 | 0.12 | 0.12 | 0.80 | 0.67 | 0.12 | 5.27 | 1 |

TABLE 2-2
T₃NO vs. BPH: Gene upregulated 5 fold or higher in at
least 2 tumor samples or 3 fold or higher in at least 3 tumor samples.

| | | Gen Bank | | Secreted/ | | | Fold/ | Fold | Fold | Fold | Fold | Fold | Fold | # of |
|---|---|---|---|---|---|---|---|---|---|---|---|---|---|---|
| | IMAGE | Accession | Tissue | Membrane | | | (Ave Tumor/A | (MPM 120/A | (MPM 121/A | (MPM 124/A | (MPM 122/A | (MPM 123/A | (MPM 125/A | Tumors |
| Order | Clone ID | Number | Distribution | Bound | AveBPH | AveTumor | vsBPH) | vsBPH) | vsBPH) | vsBPH) | vsBPh) | vsBPH) | vsBPH) | Samples |
| 1 | 788180 | AA453310 | prostate | | 1.96 | 133.47 | 68 | 149.63 | 185.70 | 7.38 | 62.32 | 22.54 | 10.38 | 6 |
| 2 | 646037 | AA196979 | prostate | | 2.38 | 114.00 | 48 | 85.24 | 24.02 | 77.47 | 31.06 | 53.59 | 35.00 | 6 |
| 3 | 284701 | N864840 | prostate | Y | 2.41 | 73.10 | 30 | 31.27 | 50.07 | 34.87 | 5.28 | 41.24 | 19.50 | 6 |
| 4 | 450049 | AA703396 | prostate | N/A | 2.55 | 76.64 | 30 | 38.52 | 11.57 | 48.94 | 15.57 | 45.42 | 20.45 | 6 |
| 5 | 245768 | N55266 | — | | 0.01 | 0.21 | 22 | 11.34 | 8.80 | 10.00 | 19.72 | 21.41 | 63.81 | 6 |
| 6 | 281003 | N50880 | t-cell | | 24.05 | 423.65 | 18 | 22.19 | 20.64 | 22.40 | 15.57 | 11.61 | 13.29 | 6 |
| 7 | 136606 | R35051 | | Y | 2.56 | 44.38 | 17 | 29.78 | 41.52 | 9.28 | 10.57 | 5.71 | 7.21 | 6 |
| 8 | 133130 | R28397 | prostate | Y | 1.48 | 25.48 | 17 | 30.48 | 41.34 | 6.15 | 9.24 | 10.23 | 5.98 | 6 |
| 9 | 836688 | AA457235 | prostate | | 3.44 | 48.10 | 13 | 11.55 | 50.28 | 3.85 | 4.31 | 3.32 | 7.01 | 6 |
| 10 | 179753 | H51549 | bone | | 0.01 | 0.12 | 12 | 9.28 | 11.64 | 11.90 | 10.01 | 17.09 | 10.66 | 6 |
| 11 | 1030855 | AA821761 | — | Y | 1.24 | 12.57 | 10 | 7.99 | 15.61 | 11.93 | 6.14 | 4.27 | 15.03 | 6 |
| 12 | 514338 | H20747 | — | | 2.07 | 19.56 | 9 | 6.78 | 6.32 | 6.58 | 7.12 | 8.55 | 19.24 | 6 |
| 13 | 306068 | N91003 | | Y | 0.80 | 6.94 | 9 | 13.54 | 16.10 | 7.85 | 4.72 | 3.65 | 6.31 | 6 |
| 14 | 195976 | R91396 | — | | 0.50 | 3.83 | 8 | 5.89 | 8.60 | 8.91 | 5.51 | 12.71 | 6.41 | 6 |
| 15 | 219311 | T2543 | breast | | 0.29 | 1.97 | 7 | 3.24 | 10.05 | 6.60 | 7.18 | 0.75 | 6.20 | 6 |
| 16 | 292217 | N79180 | bone | | 0.06 | 0.42 | 7 | 3.94 | 10.11 | 7.14 | 10.04 | 4.07 | 5.11 | 6 |
| 17 | 625859 | AA504764 | — | N/A | 1.91 | 12.19 | 6 | 6.28 | 4.64 | 5.38 | 5.13 | 5.23 | 11.57 | 6 |
| 18 | 428816 | A4004681 | — | | 0.10 | 0.60 | 6 | 6.54 | 6.44 | 8.60 | 4.79 | 4.07 | 3.73 | 6 |
| 19 | 195034 | R88764 | | | 0.02 | 0.14 | 8 | 3.19 | 6.01 | 6.21 | 7.48 | 3.67 | 8.48 | 6 |
| 20 | 359701 | AA011170 | — | | 2.91 | 12.87 | 4 | 3.21 | 3.87 | 3.89 | 6.96 | 3.53 | 3.06 | 6 |
| 21 | 243641 | N49548 | — | Maybe | 1.55 | 6.34 | 4 | 3.17 | 5.98 | 4.18 | 3.84 | 3.52 | 3.85 | 6 |
| 22 | 262035 | H98688 | — | | 0.22 | 0.88 | 4 | 3.77 | 4.13 | 3.55 | 4.47 | 3.77 | 4.01 | 8 |
| 23 | 287528 | N62096 | — | | 0.30 | 3.88 | 13 | 15.20 | 2.45 | 18.94 | 12.53 | 19.49 | 9.01 | 5 |
| 24 | 784188 | AA4432103 | prostate | | 16.97 | 194.72 12 | 9.82 | 26.16 | 14.34 | 17.70 | 1.22 | 3.92 | 5 | |
| 25 | 250374 | N31982 | prostate | | 1.29 | 13.52 | 10 | 7.91 | 24.39 | 10.24 | 5.27 | 13.26 | 1.75 | 5 |
| 26 | 210820 | H67712 | — | | 0.19 | 1.82 | 10 | 2.75 | 9.06 | 3.94 | 3.06 | 33.96 | 4.88 | 5 |
| 27 | 193923 | R83407 | brain | Y | 17.50 | 158.73 | 9 | 10.67 | 18.00 | 7.67 | 13.10 | 1.22 | 3.27 | 5 |
| 28 | 37980 | R81372 | lung | | 0.09 | 0.74 | 8 | 9.48 | 26.73 | 3.79 | 3.02 | 4.81 | 0.54 | 5 |
| 29 | 1031590 | AA409485 | — | | 0.27 | 2.07 | 8 | 5.69 | 10.96 | 6.72 | 12.64 | 2.83 | 7.06 | 5 |
| 30 | 881607 | T2336 | bone | | 1.71 | 11.25 | 7 | 7.39 | 5.16 | 4.03 | 15.83 | 2.68 | 4.49 | 5 |
| 31 | 268211 | N29966 | prostate | | 0.57 | 3.71 | 6 | 8.59 | 3.86 | 13.77 | 2.12 | 4.38 | 6.18 | 5 |
| 32 | 462486 | AA699895 | — | N/A | 0.38 | 2.39 | 8 | 5.95 | 10.82 | 5.48 | 10.02 | 1.94 | 3.81 | 5 |
| 33 | 666276 | AA663160 | — | N/A | 3.26 | 20.49 | 6 | 1.91 | 3.57 | 4.27 | 14.38 | 9.64 | 3.94 | 5 |
| 34 | 811682 | AA454597 | bone | Y | 3.59 | 21.22 | 6 | 9.12 | 4.48 | 4.71 | 6.04 | 2.69 | 6.45 | 5 |
| 35 | 428436 | A4004412 | — | | 0.53 | 3.12 | 6 | 1.13 | 4.65 | 4.73 | 5.71 | 11.11 | 7.68 | 5 |
| 36 | 130136 | R20613 | — | | 0.04 | 0.20 | 6 | 7.23 | 6.80 | 5.30 | 5.38 | 6.96 | 2.06 | 5 |
| 37 | 347566 | W861432 | heart | | 0.04 | 0.24 | 6 | 2.99 | 4.97 | 4.10 | 6.44 | 3.72 | 11.48 | 5 |
| 38 | 795729 | AA460291 | unspecified | | 0.02 | 0.11 | 8 | 6.89 | 4.96 | 0.83 | 11.93 | 6.34 | 3.73 | 5 |
| 39 | 825785 | AA505117 | ovary | | 1.58 | 8.57 | 8 | 3.24 | 6.98 | 2.95 | 6.31 | 9.01 | 4.86 | 5 |
| 40 | 498581 | H29290 | brain | | 0.33 | 1.80 | 5 | 2.31 | 3.63 | 6.60 | 3.61 | 13.00 | 3.10 | 5 |
| 41 | 417075 | W47823 | — | | 0.83 | 4.38 | 5 | 2.79 | 3.52 | 3.88 | 4.21 | 8.00 | 0.54 | 5 |
| 42 | 840751 | AA486071 | — | Y | 1.25 | 0.00 | 5 | 3.31 | 10.20 | 3.87 | 2.36 | 7.58 | 4.13 | 5 |
| 43 | 241521 | H93381 | — | N/A | 1.06 | 5.43 | 5 | 3.95 | 6.61 | 5.92 | 6.06 | 2.79 | 3.31 | 5 |
| 44 | 29063 | R40070 | prostate | | 1.68 | 8.52 | 5 | 4.25 | 6.83 | 4.57 | 7.80 | 4.02 | 2.43 | 5 |
| 45 | 124128 | R01348 | — | Y | 1.25 | 0.16 | 5 | 2.53 | 5.00 | 3.89 | 4.48 | 6.48 | 5.11 | 5 |
| 46 | 238821 | H65030 | lymph | | 4.70 | 23.13 | 5 | 2.33 | 3.01 | 3.66 | 11.43 | 4.02 | 4.20 | 5 |
| 47 | 134719 | R28287 | colon | | 0.29 | 1.40 | 5 | 5.02 | 7.58 | 4.08 | 5.94 | 3.53 | 2.93 | 5 |
| 48 | 267745 | N62244 | breast | | 3.81 | 18.41 | 5 | 2.38 | 4.28 | 3.75 | 9.84 | 3.82 | 5.12 | 5 |
| 49 | 271141 | AA34482 | — | | 0.01 | 0.06 | 5 | 0.79 | 8.80 | 4.10 | 3.79 | 5.37 | 4.56 | 5 |
| 50 | 1240414 | AA788780 | — | | 0.79 | 3.60 | 5 | 1.88 | 4.13 | 3.48 | 7.19 | 6.34 | 4.47 | 5 |
| 51 | 307240 | N93436 | testis | | 3.38 | 15.26 | 5 | 9.25 | 5.31 | 0.40 | 5.38 | 3.29 | 3.63 | 5 |
| 52 | 783848 | AA443688 | ovary | | 0.13 | 0.59 | 4 | 4.82 | 5.19 | 8.45 | 4.67 | 1.71 | 4.04 | 5 |
| 53 | 697720 | M598982 | brain | | 0.32 | 1.38 | 4 | 3.68 | 3.50 | 2.94 | 3.83 | 6.48 | 5.83 | 5 |
| 54 | 283070 | N51297 | testis | | 0.03 | 0.12 | 4 | 3.87 | 0.96 | 3.48 | 5.50 | 1.54 | 4.45 | 6 |
| 55 | 265005 | N21061 | skin | | 0.05 | 0.20 | 4 | 8.18 | 3.28 | 3.22 | 6.00 | 0.56 | 6.16 | 5 |
| 56 | 343087 | W70234 | bone | | 8.51 | 35.37 | 4 | 8.18 | 7.98 | 3.20 | 3.85 | 0.24 | 3.50 | 5 |
| 57 | 416655 | W67251 | — | Y | 0.46 | 1.88 | 4 | 2.15 | 7.06 | 3.45 | 3.00 | 8.14 | 3.31 | 5 |
| 58 | 642840 | AA486281 | lung | Y | 1.15 | 4.82 | 4 | 3.49 | 4.03 | 2.84 | 4.44 | 5.85 | 3.05 | 5 |
| 59 | 40890 | R55882 | brain | | 0.05 | 0.18 | 4 | 3.26 | 3.93 | 4.80 | 3.68 | 4.50 | 2.31 | 5 |
| 60 | 350661 | AA011095 | prostate | | 23.32 | 84.82 | 4 | 4.70 | 6.58 | 3.16 | 3.29 | 3.49 | 0.84 | 5 |
| 61 | 126230 | R06307 | brain | | 0.03 | 0.10 | 3 | 3.19 | 3.38 | 3.88 | 4.68 | 4.90 | 0.89 | 5 |
| 62 | 575682 | AA032221 | prostate | Y | 15.88 | 45.43 | 3 | 3.03 | 3.29 | 3.51 | 3.03 | 3.10 | 1.41 | 6 |
| 63 | 1486260 | AA884926 | — | N/A | 0.65 | 22.38 | 34 | 18.89 | 1.89 | 59.34 | 105.24 | 0.80 | 19.00 | 4 |
| 64 | 743828 | AA634379 | bone | N/A | 0.56 | 12.90 | 22 | 29.78 | 48.11 | 20.56 | 0.88 | 23.82 | 1.26 | 4 |
| 65 | 1390884 | AA43592 | prostate | | 1.84 | 33.00 | 18 | 49.03 | 41.43 | 3.22 | 1.15 | 2.77 | 3.51 | 4 |
| 66 | 41847 | R52794 | brain | | 0.40 | 6.34 | 18 | 9.65 | 9.61 | 1.44 | 57.03 | 15.40 | 1.72 | 4 |
| 67 | 744944 | AA825890 | prostate | | 1.90 | 22.42 | 12 | 2.78 | 4.13 | 1.88 | 6.52 | 48.21 | 5.35 | 4 |
| 68 | 1034473 | AA770728 | prostate | N/A | 0.13 | 1.38 | 11 | 22.49 | 23.74 | 3.29 | 9.25 | 2.32 | 1.91 | 4 |
| 89 | 487165 | AA045074 | colon | | 0.01 | 0.00 | 9 | 13.49 | 10.45 | 13.83 | 13.97 | 1.00 | 1.00 | 4 |
| 70 | 1458424 | AA862992 | breast | | 0.06 | 0.69 | 8 | 10.16 | 21.53 | 1.58 | 8.31 | 4.84 | 2.76 | 4 |
| 71 | 360155 | AA013260 | | | 0.01 | 0.11 | 8 | 10.07 | 0.73 | 2.20 | 24.39 | 4.57 | 6.15 | 4 |
| 72 | 144740 | R76229 | prostate | | 0.19 | 1.27 | 7 | 7.58 | 10.38 | 8.62 | 10.48 | 1.84 | 2.83 | 4 |
| 73 | 24915 | R39066 | — | | 0.93 | 5.97 | 6 | 3.07 | 2.31 | 4.37 | 2.85 | 10.25 | 15.87 | 4 |
| 74 | 593185 | AA165678 | | V | 9.70 | 60.24 | 6 | 9.37 | 12.99 | 2.48 | 6.80 | 2.35 | 3.29 | 4 |
| 75 | 1056172 | AA520005 | muscle | | 11.61 | 70.00 | 8 | 8.88 | 5.08 | 10.11 | 2.19 | 1.74 | 10.42 | 4 |
| 76 | 782383 | AA431407 | — | | 0.95 | 5.55 | 8 | 8.95 | 11.77 | 5.12 | 5.02 | 1.51 | 2.87 | 4 |
| 77 | 1031113 | AA809014 | — | | 0.01 | 0.07 | 8 | 18.58 | 0.83 | 6.44 | 4.81 | 2.05 | 3.19 | 4 |
| 78 | 481841 | AA705258 | — | N/A | 0.34 | 1.88 | 5 | 2.80 | 6.47 | 9.51 | 0.43 | 5.07 | 2.77 | 4 |
| 79 | 491706 | AA115259 | breast | | 0.75 | 3.90 | 5 | 3.93 | 8.17 | 7.31 | 9.02 | 2.29 | 2.67 | 4 |
| 80 | 787938 | AA452270 | prostate | Y | 1.76 | 8.87 | 5 | 8.20 | 9.84 | 6.65 | 1.30 | 0.26 | 5.98 | 4 |
| 81 | 754034 | AA479058 | — | | 0.02 | 0.10 | 5 | 5.74 | 6.67 | 0.33 | 1.52 | 8.64 | 9.17 | 4 |
| 82 | 070500 | AA663077 | bone | | 0.04 | 0.18 | 5 | 3.02 | 13.76 | 4.92 | 2.90 | 1.71 | 3.26 | 4 |
| 83 | 88767 | T53389 | bone | | 0.02 | 0.06 | 5 | 1.60 | 3.59 | 10.99 | 8.52 | 0.64 | 4.09 | 4 |
| 84 | 292515 | N68465 | muscle | | 3.53 | 17.34 | 5 | 3.22 | 3.11 | 2.03 | 11.89 | 6.25 | 2.97 | 4 |
| 85 | 296210 | N74392 | t-cell | | 0.44 | 2.17 | 5 | 3.10 | 3.94 | 6.60 | 2.56 | 2.70 | 10.43 | 4 |
| 86 | 40402 | R52069 | hypothalamus | | 0.02 | 0.09 | 5 | 3.44 | 0.53 | 4.63 | 7.12 | 1.57 | 11.85 | 4 |
| 87 | 656153 | AA633611 | heart | | 0.05 | 0.24 | 5 | 1.99 | 4.80 | 2.74 | 9.26 | 4.42 | 5.71 | 4 |
| 88 | 6704597 | AA262253 | breast | | 0.27 | 1.26 | 5 | 1.92 | 9.73 | 7.63 | 3.55 | 3.08 | 1.23 | 4 |
| 89 | 614306 | AA459318 | prostate | | 9.65 | 45.42 | 5 | 5.23 | 10.10 | 4.41 | 5.26 | 1.16 | 2.07 | 4 |
| 90 | 761233 | AA448316 | — | | 0.02 | 0.10 | 5 | 0.48 | 4.63 | 6.36 | 7.51 | 1.30 | 7.69 | 4 |
| 91 | 823794 | AA490283 | colon | | 1.70 | 7.94 | 5 | 3.60 | 10.81 | 5.89 | 3.38 | 1.65 | 2.40 | 4 |
| 92 | 260170 | N32072 | prostate | | 12.65 | 59.20 | 5 | 5.52 | 5.50 | 9.33 | 1.01 | 1.63 | 4.68 | 4 |
| 93 | 449309 | AA777902 | | N/A | 2.47 | 11.22 | 5 | 4.85 | 3.66 | 3.67 | 9.59 | 2.36 | 2.67 | 4 |
| 94 | 230155 | H461756 | — | Maybe | 0.96 | 4.35 | 5 | 4.04 | 2.42 | 10.26 | 1.76 | 4.61 | 3.79 | 4 |
| 95 | 296079 | W2258 | unspecified | | 0.94 | 4.15 | 4 | 1.85 | 2.47 | 3.73 | 5.40 | 9.60 | 3.44 | 4 |
| 96 | 195387 | R66915 | — | | 0.47 | 2.07 | 4 | 5.34 | 6.40 | 6.39 | 1.35 | 5.02 | 1.71 | 4 |
| 97 | 428267 | AA004026 | unspecified | | 1.41 | 6.20 | 4 | 2.17 | 5.43 | 7.71 | 1.35 | 3.96 | 5.65 | 4 |
| 98 | 391964 | AJ003625 | — | | 4.42 | 19.15 | 4 | 3.25 | 3.54 | 4.23 | 10.05 | 2.96 | 1.97 | 4 |
| 99 | 5170014 | H22654 | brain | | 1.92 | 8.25 | 4 | 5.19 | 4.37 | 3.15 | 9.71 | 1.61 | 1.53 | 4 |
| 100 | 764204 | AA479494 | breast | Y | 15.01 | 62.10 | 4 | 5.48 | 5.99 | 2.50 | 3.28 | 4.67 | 2.90 | 4 |
| 101 | 210697 | H683840 | colon | Maybe | 0.75 | 3.08 | 4 | 1.97 | 3.35 | 4.69 | 7.43 | 6.18 | 1.26 | 4 |
| 102 | 809456 | AA443090 | bone | | 0.02 | 0.10 | 4 | 6.87 | 3.20 | 5.81 | 2.50 | 1.75 | 4.48 | 4 |
| 103 | 361659 | W98189 | skin | Maybe | 6.37 | 26.06 | 4 | 2.86 | 6.98 | 3.63 | 4.72 | 4.26 | 2.07 | 4 |
| 104 | 148225 | H13668 | bone | | 1.72 | 7.01 | 4 | 3.68 | 2.48 | 4.06 | 2.17 | 6.06 | 3.94 | 4 |
| 105 | 417867 | W90128 | breast | | 13.56 | 54.72 | 4 | 3.89 | 3.21 | 2.71 | 10.52 | 0.69 | 3.20 | 4 |
| 100 | 436531 | AA703048 | — | N/A | 1.44 | 5.61 | 4 | 1.27 | 3.01 | 3.96 | 5.13 | 6.53 | 2.31 | 4 |
| 107 | 1031348 | AA009106 | hypothalamus | | 1.74 | 6.94 | 4 | 6.11 | 5.02 | 3.69 | 2.74 | 2.97 | 3.41 | 4 |
| 108 | 1567933 | AA939275 | brain | N/A | 0.61 | 2.36 | 4 | 2.08 | 5.00 | 4.77 | 3.69 | 6.17 | 1.75 | 4 |
| 109 | 144747 | R76247 | brain | | 0.43 | 1.67 | 4 | 9.05 | 3.34 | 1.44 | 1.37 | 4.41 | 3.71 | 4 |
| 110 | 321386 | WJ2272 | t-cell | | 1.23 | 4.75 | 4 | 5.19 | 5.74 | 3.61 | 2.10 | 3.36 | 2.84 | 4 |
| 111 | 725265 | AA291742 | t-cell | | 10.05 | 0.19 | 4 | 2.57 | 4.47 | 5.19 | 3.29 | 7.01 | 0.16 | 4 |
| 112 | 258404 | N25663 | — | | 0.28 | 1.07 | 4 | 6.40 | 3.53 | 3.79 | 6.85 | 1.28 | 0.83 | 4 |
| 113 | 266692 | N25920 | — | | 1.61 | 6.68 | 4 | 2.93 | 4.16 | 2.58 | 3.38 | 4.60 | 4.50 | 4 |
| 114 | 283995 | N53376 | brain | | 0.93 | 3.42 | 4 | 2.34 | 3.33 | 3.82 | 2.44 | 4.10 | 5.95 | 4 |
| 115 | 359781 | AA011320 | colon | | 3.58 | 13.06 | 4 | 4.87 | 5.61 | 2.55 | 4.74 | 3.65 | 0.49 | 4 |
| 116 | 664701 | AA630104 | bone | Y | 0.96 | 3.56 | 4 | 2.92 | 5.15 | 3.38 | 5.17 | 4.30 | 1.00 | 4 |
| 117 | 24886 | H482061 | breast | | 0.83 | 3.03 | 4 | 4.16 | 2.79 | 3.62 | 8.06 | 3.32 | 2.93 | 4 |
| 118 | 771053 | AA427528 | prostate | | 10.52 | 36.08 | 4 | 3.55 | 5.47 | 3.81 | 5.36 | 2.39 | 1.11 | 4 |
| 119 | 488010 | AA045709 | Neuron | | 0.96 | 3.54 | 4 | 1.73 | 6.29 | 3.11 | 1.20 | 3.29 | 5.98 | 4 |
| 120 | 11242 | T90980 | bone | N/A | 11.33 | 40.58 | 4 | 1.21 | 4.61 | 5.01 | 4.80 | 4.57 | 1.26 | 4 |
| 121 | 86005 | T62842 | breast | | 0.03 | 0.10 | 4 | 5.81 | 3.02 | 3.15 | 2.19 | 2.76 | 4.13 | 4 |
| 122 | 855632 | AA864104 | — | N/A | 0.96 | 3.36 | 4 | 2.09 | 3.77 | 3.88 | 1.92 | 5.82 | 3.68 | 4 |
| 123 | 611914 | AA454988 | — | N/A | 2.96 | 10.39 | 4 | 3.92 | 5.10 | 3.87 | 3.79 | 2.79 | 1.82 | 4 |
| 124 | 281371 | N47886 | bone | | 0.38 | 1.25 | 3 | 5.39 | 3.04 | 3.53 | 2.17 | 2.42 | 4.40 | 4 |
| 125 | 383823 | AA704850 | | Y | 9.01 | 31.38 | 3 | 2.44 | 3.12 | 4.85 | 1.73 | 3.48 | 5.29 | 4 |
| 128 | 395400 | AA757401 | bone | | 2.20 | 7.88 | 3 | 2.26 | 5.31 | 3.00 | 4.08 | 4.15 | 2.06 | 4 |
| 127 | 138507 | R63253 | — | Y | 1.41 | 4.88 | 3 | 1.74 | 4.81 | 3.90 | 3.23 | 4.16 | 2.95 | 4 |
| 128 | 289071 | N83609 | — | Y | 19.85 | 88.00 | 3 | 3.37 | 5.09 | 1.38 | 1.59 | 6.09 | 3.28 | 4 |
| 129 | 480203 | AA877481 | — | N/A | 0.55 | 1.89 | 3 | 1.61 | 4.23 | 4.13 | 4.28 | 4.93 | 1.55 | 4 |
| 130 | 242061 | H93335 | testis | | 2.71 | 9.21 | 3 | 5.12 | 6.73 | 3.43 | 3.25 | 0.92 | 0.95 | 4 |
| 131 | 207098 | H48502 | embryo | | 0.16 | 0.53 | 3 | 3.02 | 4.77 | 3.04 | 2.28 | 4.85 | 2.52 | 4 |
| 132 | 396839 | AA758154 | colon | | 0.88 | 2.24 | 3 | 1.87 | 5.82 | 3.24 | 4.80 | 1.60 | 3.25 | 4 |
| 133 | 209381 | H84095 | colon | Y | 0.97 | 3.25 | 3 | 2.04 | 3.28 | 4.07 | 1.88 | 4.90 | 3.98 | 4 |
| 134 | 23897 | R38364 | — | | 0.38 | 1.24 | 3 | 1.98 | 3.91 | 5.80 | 3.28 | 1.47 | 3.16 | 4 |
| 135 | 1487905 | AA883504 | thymus | N/A | 1.18 | 3.84 | 3 | 1.67 | 3.82 | 4.55 | 3.37 | 3.97 | 2.04 | 4 |
| 136 | 1558526 | AA935560 | prostate | N/A | 1.17 | 3.77 | 3 | 3.58 | 5.88 | 3.20 | 0.99 | 4.48 | 1.12 | 4 |
| 137 | 491418 | AA115537 | breast | | 0.84 | 1.72 | 3 | 1.82 | 3.32 | 2.48 | 4.38 | 3.33 | 3.88 | 4 |
| 138 | 744001 | AA629039 | heart | N/A | 3.87 12.24 | 3 | 1.80 | 3.05 | 3.27 | 4.18 | 5.11 | 1.80 | 4 | |
| 139 | 2931101 | N83848 | — | | 3.38 10.70 | 3 | 2.38 | 3.94 | 3.44 | 3.88 | 3.55 | 1.84 | 4 | |
| 140 | 782047 | AA29802 | prostate | Maybe | 1.17 | 3.67 | 3 | 3.26 | 2.51 | 3.73 | 2.89 | 3.22 | 3.21 | 4 |
| 141 | 454889 | AA677206 | — | | 1.02 | 3.18 | 3 | 2.09 | 3.15 | 1.89 | 3.87 | 3.10 | 4.59 | 4 |
| 142 | 384293 | AA709370 | — | N/A | 0.50 | 1.53 | 3 | 4.26 | 3.84 | 4.05 | 3.20 | 1.06 | 1.87 | 4 |
| 143 | 34091 | R44132 | brain | | 0.53 | 1.63 | 3 | 2.04 | 3.94 | 3.79 | 3.14 | 3.71 | 1.62 | 4 |
| 144 | 743362 | AA400385 | prostate | | 0.03 | 0.09 | 3 | 3.33 | 3.10 | 2.05 | 4.16 | 0.88 | 4.58 | 4 |
| 145 | 126785 | R10015 | heart | Y | 1.14 | 3.44 | 3 | 2.11 | 2.81 | 3.08 | 3.17 | 3.53 | 3.40 | 4 |
| 148 | 429299 | AA007370 | breast | | 1.26 | 3.83 | 3 | 3.22 | 3.29 | 1.89 | 3.35 | 2.22 | 3.94 | 4 |
| 147 | 138819 | R38485 | — | | 0.47 | 1.37 | 3 | 3.15 | 2.95 | 3.25 | 3.12 | 1.85 | 3.33 | 4 |
| 148 | 433350 | AA700604 | | | 42.83 | 123.18 | 3 | 3.88 | 5.22 | 3.12 | 1.22 | 0.47 | 3.55 | 4 |
| 149 | 755278 | AA496349 | brain | | 0.11 | 0.50 | 3 | 3.57 | 4.62 | 3.03 | 1.81 | 0.38 | 3.05 | 4 |
| 150 | 270917 | N32514 | | | 0.01 | 0.23 | 28 | 3.34 | 1.13 | 1.13 | 14.34 | 134.44 | 1.13 | 3 |
| 151 | 762233 | AA431721 | prostate | Y | 0.20 | 3.10 | 15 | 64.92 | 1.22 | 0.11 | 0.61 | 3.63 | 22.02 | 3 |
| 152 | 320495 | W16659 | bone | | 0.06 | 0.88 | 14 | 1.39 | 3.75 | 0.97 | 71.38 | 3.78 | 2.68 | 3 |
| 153 | 293679 | N94111 | spleen | | 0.96 | 12.90 | 13 | 0.73 | 1.52 | 4.77 | 28.47 | 44.95 | 2.05 | 3 |
| 154 | 281039 | N47717 | endothelium | | 0.78 | 9.99 | 13 | 43.18 | 25.22 | 5.01 | 2.22 | 0.30 | 1.22 | 3 |
| 155 | 490718 | AA115761 | embryo | | 0.04 | 0.48 | 12 | 46.34 | 8.67 | 1.63 | 0.27 | 18.31 | 0.82 | 3 |
| 156 | 798747 | AA443147 | bone | | 0.06 | 0.92 | 11 | 0.12 | 0.12 | 2.23 | 55.00 | 5.81 | 3.44 | 3 |
| 157 | 287728 | N59188 | — | V | 0.51 | 5.43 | 11 | 20.89 | 31.93 | 7.81 | 1.25 | 0.88 | 1.67 | 3 |
| 158 | 282089 | N48259 | prostate | | 1.90 | 19.34 | 10 | 21.89 | 29.40 | 4.51 | 1.88 | 1.07 | 2.28 | 3 |
| 159 | 731290 | AA416627 | breast | | 4.06 33.97 | 8 | 14.84 | 26.99 | 1.81 | 1.28 | 0.78 | 4.21 | 3 | |
| 160 | 470216 | AA028987 | prostate | | 2.95 | 23.62 | 8 | 1.97 | 4.75 | 1.06 | 5.39 | 32.28 | 2.88 | 3 |
| 161 | 375827 | AA039851 | heart | | 0.07 | 0.57 | 8 | 3.54 | 3.42 | 1.63 | 1.38 | 34.08 | 2.42 | 3 |
| 162 | 277714 | N49577 | — | | 0.13 | 0.94 | 7 | 0.87 | 3.63 | 1.15 | 32.30 | 5.68 | 0.12 | 3 |
| 163 | 757451 | AA437241 | — | N/A | 0.47 | 3.37 | 7 | 4.87 | 30.78 | 1.10 | 3.88 | 0.99 | 1.38 | 3 |
| 164 | 486035 | AA040801 | muscle | N/A | 5.25 | 35.89 | 7 | 2.41 | 4.29 | 2.60 | 21.59 | 8.58 | 1.59 | 3 |
| 165 | 298417 | N74131 | colon | | 0.08 | 0.48 | 8 | 13.15 | 18.83 | 1.72 | 1.58 | 1.47 | 3.24 | 3 |
| 166 | 668218 | AA233809 | skin | | 0.04 | 0.23 | 6 | 1.91 | 7.98 | 4.25 | 20.28 | 2.26 | 0.61 | 3 |
| 167 | 757389 | AA437142 | — | | 0.02 | 0.11 | 6 | 1.70 | 4.23 | 0.65 | 4.78 | 0.58 | 22.88 | 3 |
| 168 | 196168 | R92382 | — | | 0.59 | 3.32 | 8 | 1.71 | 1.85 | 2.16 | 16.65 | 4.00 | 7.34 | 3 |
| 169 | 687667 | AA235370 | hypothalamus | | 0.81 | 4.88 | 8 | 5.88 | 18.69 | 4.47 | 1.32 | 1.05 | 2.26 | 3 |
| 170 | 712050 | AA282273 | t-cell | | 8.93 | 32.98 | 8 | 8.81 | 14.24 | 8.62 | 0.72 | 0.88 | 2.12 | 3 |
| 171 | 504761 | AA148735 | bone | | 0.08 | 0.43 | 5 | 8.28 | 0.51 | 7.72 | 0.13 | 17.97 | 0.10 | 3 |
| 172 | 322443 | W16424 | adrenal | | 0.23 | 1.23 | 5 | 3.13 | 4.09 | 0.73 | 20.38 | 0.88 | 2.62 | 3 |
| 173 | 35010 | R45116 | — | | 0.68 | 3.31 | 5 | 2.72 | 4.85 | 2.60 | 1.65 | 11.48 | 6.88 | 3 |
| 174 | 782537 | AA444484 | testis | | 1.72 | 6.80 | 5 | 2.52 | 2.78 | 8.00 | 0.99 | 7.48 | 8.28 | 3 |
| 175 | 277596 | N57002 | bone | Y | 1.48 | 7.34 | 5 | 7.83 | 12.12 | 2.17 | 0.90 | 1.13 | 8.84 | 3 |
| 176 | 773208 | AA425850 | skin | | 9.71 | 47.09 | 5 | 1.62 | 1.90 | 6.84 | 12.13 | 2.89 | 3.52 | 3 |
| 177 | 384885 | AA035745 | breast | | 0.44 | 2.09 | 5 | 1.52 | 1.24 | 13.33 | 0.96 | 7.22 | 4.39 | 3 |
| 178 | 247587 | N58138 | bone | | 0.01 | 0.08 | 5 | 1.88 | 14.29 | 1.91 | 4.85 | 3.94 | 0.89 | 3 |
| 179 | 34888 | R19878 | — | | 0.14 | 0.87 | 5 | 5.89 | 9.05 | 6.74 | 1.93 | 1.68 | 2.65 | 3 |
| 180 | 723957 | T51539 | placenta | | 0.08 | 0.38 | 5 | 0.87 | 16.34 | 4.47 | 1.37 | 4.55 | 0.13 | 3 |
| 181 | 773204 | AA425892 | bone | | 12.74 | 58.08 | 5 | 1.47 | 1.49 | 8.37 | 13.00 | 2.45 | 3.58 | 3 |
| 182 | 277327 | N57483 | brain | | 0.02 | 0.09 | 5 | 1.87 | 6.21 | 2.65 | 8.18 | 1.05 | 7.68 | 3 |
| 183 | 770088 | AA430645 | brain | | 7.38 | 33.38 | 5 | 7.28 | 8.45 | 8.19 | 0.54 | 0.28 | 2.38 | 3 |
| 184 | 616201 | H19448 | — | | 0.03 | 0.12 | 4 | 1.90 | 8.33 | 7.29 | 8.63 | 2.43 | 0.38 | 3 |
| 185 | 268343 | N26558 | — | | 0.70 | 3.10 | 4 | 8.82 | 9.88 | 4.53 | 0.84 | 0.94 | 1.83 | 3 |
| 186 | 296170 | W02426 | — | | 0.43 | 1.87 | 4 | 4.09 | 12.69 | 6.34 | 0.91 | 1.09 | 1.27 | 3 |
| 187 | 278504 | N86139 | brain | | 0.05 | 0.22 | 4 | 0.98 | 7.98 | 1.80 | 5.23 | 1.24 | 8.83 | 3 |
| 188 | 526587 | AA128407 | breast | | 0.02 | 0.07 | 4 | 3.30 | 1.87 | 0.98 | 9.74 | 0.84 | 9.87 | 3 |
| 189 | 809526 | AA454570 | lung | | 0.06 | 0.28 | 4 | 10.35 | 0.16 | 0.26 | 2.13 | 8.88 | 4.11 | 3 |
| 190 | 292058 | N89499 | — | | 0.05 | 0.22 | 4 | 0.48 | 1.96 | 0.88 | 3.72 | 15.21 | 3.80 | 3 |
| 191 | 295997 | N73563 | — | | 0.42 | 1.81 | 4 | 7.32 | 9.76 | 3.67 | 2.18 | 1.01 | 1.74 | 3 |
| 192 | 205828 | H57585 | prostate | | 0.81 | 2.18 | 4 | 7.69 | 2.40 | 5.57 | 1.28 | 1.92 | 6.79 | 3 |
| 193 | 873977 | T49325 | testis | | 0.01 | 0.08 | 4 | 5.29 | 7.99 | 0.88 | 0.90 | 0.68 | 9.45 | 3 |
| 194 | 811028 | AA485373 | breast | | 4.96 | 20.42 | 4 | 3.45 | 0.81 | 2.68 | 7.58 | 1.37 | 8.82 | 3 |
| 195 | 489837 | AA101878 | prostate | N/A | 2.68 | 11.62 | 4 | 3.07 | 1.88 | 7.47 | 8.01 | 2.44 | 1.39 | 3 |
| 196 | 284824 | N64796 | — | | 1.58 | 8.38 | 4 | 2.09 | 8.18 | 8.46 | 2.24 | 5.69 | 1.52 | 3 |
| 197 | 641507 | AA487385 | lung | Y | 1.08 | 4.28 | 4 | 10.98 | 2.50 | 4.10 | 3.38 | 1.15 | 2.08 | 3 |
| 198 | 882497 | AA676804 | bone | | 0.04 | 0.14 | 4 | 0.28 | 7.63 | 0.28 | 9.35 | 0.79 | 5.34 | 3 |
| 199 | 280155 | N47012 | | | 0.08 | 0.25 | 4 | 2.52 | 2.29 | 7.11 | 2.88 | 4.99 | 4.00 | 3 |
| 200 | 1408788 | AA883735 | — | N/A | 0.45 | 1.79 | 4 | 2.93 | 3.44 | 6.26 | 7.90 | 1.68 | 2.22 | 3 |
| 201 | 784589 | AA443300 | heart | | 0.03 | 0.10 | 4 | 3.05 | 1.55 | 3.64 | 1.88 | 1.93 | 11.30 | 3 |
| 202 | 723972 | AA292583 | bone | | 0.01 | 0.05 | 4 | 3.91 | 7.78 | 1.48 | 8.70 | 0.74 | 0.74 | 3 |
| 203 | 656174 | AA830620 | | | 0.03 | 0.11 | 4 | 8.41 | 6.18 | 0.98 | 0.35 | 1.33 | 8.10 | 3 |
| 204 | 782575 | AA447522 | cervix | | 0.38 | 1.39 | 4 | 8.55 | 7.01 | 2.57 | 0.65 | 0.88 | 3.47 | 3 |
| 205 | 426168 | AA001870 | bone | | 2.28 | 8.77 | 4 | 1.91 | 3.10 | 4.57 | 10.12 | 1.60 | 1.78 | 3 |
| 206 | 588826 | AA130857 | Neuron | N/A | 1.17 | 4.49 | 4 | 2.78 | 2.15 | 2.16 | 6.17 | 4.00 | 3.75 | 3 |
| 207 | 730838 | M417012 | testis | | 0.02 | 0.08 | 4 | 7.60 | 0.49 | 0.49 | 7.60 | 2.29 | 4.30 | 3 |
| 206 | 837953 | AA56578 | lung | Y | 7.85 | 29.78 | 4 | 3.78 | 5.93 | 1.10 | 2.48 | 8.88 | 2.62 | 3 |
| 200 | 503824 | A133959 | brain | | 0.12 | 0.44 | 4 | 1.62 | 0.09 | 1.78 | 8.89 | 6.74 | 6.57 | 3 |
| 210 | 208210 | H85288 | placenta | | 0.89 | 3.34 | 4 | 6.28 | 5.92 | 7.33 | 1.69 | 0.76 | 1.61 | 3 |
| 211 | 427996 | AA001841 | | | 0.11 | 0.43 | 4 | 3.08 | 1.05 | 3.00 | 3.88 | 0.51 | 11.00 | 3 |
| 212 | 123758 | R01304 | placenta | | 1.64 | 6.90 | 4 | 0.50 | 0.29 | 0.34 | 4.24 | 12.81 | 4.50 | 3 |
| 213 | 488163 | AA703249 | breast | N/A | 14.14 | 52.21 | 4 | 1.02 | 1.89 | 3.14 | 8.23 | 8.08 | 2.80 | 3 |
| 214 | 143062 | R71336 | — | | 0.44 | 1.81 | 4 | 2.83 | 6.04 | 2.57 | 2.87 | 3.32 | 4.66 | 3 |
| 215 | 131580 | R23727 | — | | 0.16 | 0.80 | 4 | 5.06 | 2.39 | 2.70 | 1.07 | 4.57 | 6.14 | 3 |
| 218 | 240151 | R82706 | bone | Maybe | 2.33 | 8.49 | 4 | 2.83 | 0.64 | 1.92 | 4.91 | 8.33 | 6.22 | 3 |
| 217 | 452116 | AA707219 | bone | | 0.44 | 1.61 | 4 | 2.41 | 7.49 | 3.48 | 2.29 | 3.82 | 2.52 | 3 |
| 216 | 201125 | R96472 | skin | N/A | 1.05 | 3.81 | 4 | 3.93 | 4.00 | 2.14 | 6.14 | 2.91 | 2.55 | 3 |
| 219 | 128713 | R07115 | — | | 0.27 | 0.98 | 4 | 6.84 | 4.18 | 2.20 | 4.29 | 1.20 | 2.78 | 3 |
| 220 | 768179 | AA424649 | brain | N/A | 2.58 | 9.08 | 4 | 3.04 | 3.99 | 1.69 | 2.78 | 7.17 | 2.61 | 3 |
| 221 | 250676 | H95976 | lung | Maybe | 4.09 | 14.42 | 4 | 2.95 | 6.36 | 3.23 | 3.77 | 2.33 | 2.51 | 3 |
| 222 | 273499 | N33263 | — | | 0.09 | 0.32 | 4 | 3.88 | 2.87 | 2.75 | 6.21 | 4.11 | 1.47 | 3 |
| 223 | 765765 | AA498465 | muscle | | 1.95 | 6.84 | 4 | 2.12 | 3.80 | 3.48 | 7.92 | 1.94 | 1.82 | 3 |
| 224 | 615047 | AA485188 | bone | | 2.68 | 9.34 | 3 | 2.62 | 1.80 | 1.82 | 4.71 | 3.01 | 7.18 | 3 |
| 225 | 384870 | AA700333 | | N/A | 4.17 | 14.53 | 3 | 3.11 | 2.41 | 3.77 | 7.25 | 2.34 | 2.03 | 3 |
| 226 | 782200 | AA431964 | muscle | | 1.53 | 8.30 | 3 | 2.48 | 6.32 | 2.54 | 3.94 | 2.28 | 3.29 | 3 |
| 227 | 527301 | H29500 | brain | | 6.64 | 22.94 | 3 | 2.33 | 2.11 | 4.07 | 6.25 | 3.48 | 2.49 | 3 |
| 228 | 125311 | R05810 | lung | N/A | 1.92 | 6.62 | 3 | 1.07 | 2.84 | 3.69 | 6.68 | 4.33 | 2.05 | 3 |
| 229 | 770680 | AA434404 | adrenal | | 6.05 | 27.68 | 3 | 6.26 | 3.15 | 7.67 | 1.10 | 0.49 | 1.77 | 3 |
| 230 | 798268 | AA480627 | breast | | 0.65 | 1.89 | 3 | 1.43 | 3.88 | 2.58 | 5.27 | 5.68 | 1.71 | 3 |
| 231 | 204148 | H55920 | prostate | N/A | 2.07 | 7.07 | 3 | 2.91 | 3.75 | 7.21 | 4.16 | 1.12 | 1.38 | 3 |
| 232 | 611373 | AA176633 | lung | | 0.01 | 0.04 | 3 | 9.82 | 3.38 | 1.53 | 3.98 | 0.90 | 0.90 | 3 |
| 233 | 325380 | AA284285 | spleen | | 0.01 | 0.03 | 3 | 1.88 | 1.13 | 5.78 | 5.41 | 1.13 | 6.09 | 3 |
| 234 | 434822 | AA703114 | — | | 0.60 | 2.72 | 3 | 5.38 | 1.97 | 4.32 | 1.12 | 1.56 | 6.01 | 3 |
| 238 | 1049033 | AA776675 | heart | | 1.87 | 6.35 | 3 | 4.98 | 4.75 | 2.95 | 4.55 | 1.19 | 1.94 | 3 |
| 236 | 682483 | AA678598 | | | 2.76 | 9.30 | 3 | 1.34 | 4.88 | 2.99 | 5.61 | 3.76 | 1.72 | 3 |
| 237 | 767130 | AA424535 | kidney | | 0.49 | 1.67 | 3 | 1.02 | 0.74 | 1.14 | 3.10 | 10.17 | 4.12 | 3 |
| 236 | 126321 | R08479 | — | | 0.03 | 0.10 | 3 | 2.12 | 4.44 | 3.73 | 7.41 | 0.38 | 2.21 | 3 |
| 238 | 230440 | H76326 | — | | 0.07 | 0.22 | 3 | 1.97 | 3.57 | 3.44 | 2.89 | 6.21 | 0.15 | 3 |
| 240 | 624271 | AA181178 | bone | N/A | 6.76 | 22.81 | 3 | 3.53 | 3.85 | 2.67 | 5.02 | 2.93 | 1.99 | 3 |
| 241 | 123265 | R00264 | lymph | N/A | 2.30 | 7.72 | 3 | 4.22 | 9.13 | 4.42 | 0.68 | 0.98 | 0.68 | 3 |
| 242 | 357238 | W93638 | — | | 0.72 | 2.40 | 3 | 5.76 | 2.69 | 1.43 | 1.48 | 3.53 | 5.19 | 3 |
| 243 | 123331 | R00479 | prostate | | 1.03 | 3.43 | 3 | 2.37 | 4.72 | 6.68 | 0.96 | 3.48 | 2.80 | 3 |
| 244 | 240694 | H78134 | — | Y | 1.65 | 6.17 | 3 | 2.79 | 3.07 | 1.59 | 7.73 | 3.58 | 1.21 | 3 |
| 245 | 347162 | W80611 | brain | | 0.02 | 0.07 | 3 | 0.45 | 5.03 | 5.53 | 4.94 | 2.61 | 1.19 | 3 |
| 248 | 52092 | H23232 | prostate | | 0.03 | 0.11 | 3 | 1.94 | 4.38 | 6.28 | 4.13 | 2.87 | 0.31 | 3 |
| 247 | 180628 | H25019 | lymph | Y | 0.41 | 1.38 | 3 | 2.02 | 4.53 | 3.17 | 4.45 | 2.72 | 2.93 | 3 |
| 248 | 1505294 | AA906838 | testis | N/A | 1.11 | 3.68 | 3 | 1.98 | 2.43 | 7.21 | 1.55 | 3.26 | 3.34 | 3 |
| 249 | 262053 | H98812 | t-cell | | 7.69 | 25.26 | 3 | 2.38 | 3.42 | 3.49 | 6.75 | 1.98 | 1.66 | 3 |
| 250 | 741067 | AA476436 | bone | Y | 17.59 | 57.76 | 3 | 2.99 | 3.39 | 3.14 | 2.34 | 2.25 | 5.58 | 3 |
| 251 | 490178 | AA121266 | brain | | 3.60 | 11.40 | 3 | 2.64 | 4.34 | 2.28 | 4.85 | 1.83 | 3.72 | 3 |
| 252 | 208741 | H63175 | | Y | 0.09 | 0.30 | 3 | 2.00 | 4.61 | 3.24 | 2.64 | 2.82 | 4.27 | 3 |
| 253 | 416611 | W46466 | colon | | 3.63 | 11.85 | 3 | 2.01 | 2.14 | 2.13 | 5.36 | 4.86 | 3.07 | 3 |
| 254 | 416002 | W85881 | — | | 0.60 | 1.62 | 3 | 2.00 | 2.31 | 1.93 | 3.05 | 3.65 | 6.82 | 3 |
| 255 | 221561 | H92525 | — | | 0.55 | 1.79 | 3 | 1.18 | 1.35 | 3.60 | 1.40 | 6.94 | 5.06 | 3 |
| 256 | 223274 | H86518 | — | | 0.06 | 0.17 | 3 | 3.24 | 0.19 | 0.19 | 2.05 | 5.14 | 7.79 | 3 |
| 257 | 44387 | H06525 | brain | | 1.17 | 3.78 | 3 | 6.67 | 2.89 | 4.15 | 1.74 | 3.08 | 1.09 | 3 |
| 258 | 110787 | T90639 | brain | | 0.25 | 0.80 | 3 | 2.49 | 3.52 | 2.19 | 3.80 | 2.05 | 8.34 | 3 |
| 259 | 121948 | T97762 | brain | | 4.17 | 13.48 | 3 | 1.59 | 4.12 | 2.42 | 5.41 | 3.80 | 2.03 | 3 |
| 260 | 782812 | AA448251 | brain | | 0.06 | 0.18 | 3 | 4.87 | 5.06 | 1.22 | 2.93 | 3.17 | 2.10 | 3 |
| 261 | 145513 | R77715 | skin | | 0.67 | 2.16 | 3 | 1.90 | 4.01 | 2.67 | 3.29 | 2.99 | 4.45 | 3 |
| 262 | 435319 | AA699931 | bone | | 6.14 | 19.70 | 3 | 2.30 | 5.27 | 1.85 | 3.83 | 2.74 | 3.25 | 3 |
| 263 | 210698 | H66877 | colon | Maybe | 1.41 | 4.52 | 3 | 1.59 | 2.24 | 3.47 | 5.80 | 5.20 | 0.95 | 3 |
| 264 | 197067 | R93401 | — | | 0.36 | 1.16 | 3 | 1.88 | 1.56 | 3.78 | 3.20 | 2.11 | 6.62 | 3 |
| 265 | 416374 | W86202 | bone | Maybe | 3.18 | 10.13 | 3 | 4.89 | 3.63 | 2.43 | 2.00 | 3.26 | 2.91 | 3 |
| 266 | 1422794 | AA827405 | | N/A | 2.02 | 6.43 | 3 | 2.60 | 3.91 | 2.60 | 5.88 | 1.06 | 3.06 | 3 |
| 267 | 137017 | R35665 | bone | | 0.03 | 0.08 | 3 | 1.47 | 4.27 | 1.59 | 8.06 | 3.23 | 0.45 | 3 |
| 268 | 267634 | N34117 | breast | | 0.21 | 0.86 | 3 | 2.32 | 3.85 | 2.53 | 3.68 | 3.77 | 2.92 | 3 |
| 269 | 433253 | AA699427 | breast | | 5.61 | 17.79 | 3 | 4.18 | 4.57 | 2.43 | 1.60 | 1.72 | 4.54 | 3 |
| 270 | 1475734 | AA872692 | Neuron | N/A | 7.39 | 23.26 | 3 | 3.45 | 3.26 | 2.34 | 2.32 | 2.73 | 4.79 | 3 |
| 271 | 268876 | N26026 | — | | 0.51 | 1.59 | 3 | 1.46 | 6.62 | 3.35 | 1.36 | 3.86 | 2.19 | 3 |
| 272 | 79032 | T61899 | bone | Y | 15.17 | 47.49 | 3 | 3.68 | 4.92 | 3.96 | 1.33 | 2.11 | 2.80 | 3 |
| 273 | 771025 | AA427978 | — | | 1.12 | 3.51 | 3 | 2.45 | 3.57 | 2.85 | 2.35 | 3.41 | 4.13 | 3 |
| 274 | 811101 | AA485676 | brain | | 2.17 | 6.72 | 3 | 1.08 | 2.45 | 1.78 | 5.54 | 4.36 | 3.37 | 3 |
| 275 | 270127 | N27935 | — | | 0.66 | 2.04 | 3 | 3.94 | 3.31 | 2.15 | 1.27 | 5.53 | 2.34 | 3 |
| 276 | 448117 | AA702480 | | N/A | 2.65 | 8.16 | 3 | 3.50 | 2.77 | 3.64 | 5.73 | 1.36 | 1.27 | 3 |
| 277 | 796674 | AA460557 | t-cell | | 0.48 | 1.46 | 3 | 2.06 | 2.12 | 3.31 | 6.09 | 3.43 | 2.39 | 3 |
| 278 | 431296 | AA882631 | brain | | 0.48 | 1.47 | 3 | 1.65 | 4.32 | 2.32 | 3.60 | 1.77 | 4.45 | 3 |
| 279 | 214824 | H71242 | prostate | | 1.92 | 5.87 | 3 | 2.21 | 3.82 | 1.69 | 1.33 | 5.44 | 3.82 | 3 |
| 280 | 743323 | AA400596 | | | 0.49 | 1.60 | 3 | 4.39 | 4.19 | 1.69 | 1.95 | 2.28 | 3.78 | 3 |
| 281 | 800507 | T3245 | liver | N/A | 12.72 | 36.68 | 3 | 1.01 | 1.73 | 3.23 | 4.87 | 5.49 | 1.91 | 3 |
| 282 | 1500162 | AA88739 | kidney | N/A | 1.89 | 5.73 | 3 | 2.12 | 4.97 | 1.27 | 4.24 | 1.94 | 3.69 | 3 |
| 283 | 268234 | N29992 | lung | | 1.72 | 5.20 | 3 | 2.45 | 3.22 | 5.41 | 2.32 | 1.71 | 3.08 | 3 |
| 264 | 109623 | R96561 | — | | 0.51 | 1.54 | 3 | 2.99 | 4.10 | 3.93 | 3.02 | 2.70 | 1.44 | 3 |
| 285 | 41305 | R56877 | | N/A | 19.63 | 60.07 | 3 | 2.41 | 3.80 | 2.96 | 4.40 | 3.55 | 1.06 | 3 |
| 286 | 795555 | AA459679 | colon | | 1.83 | 4.94 | 3 | 3.06 | 5.11 | 2.63 | 4.91 | 1.14 | 1.32 | 3 |
| 287 | 416808 | W66779 | — | | 0.57 | 1.71 | 3 | 1.77 | 4.00 | 2.37 | 4.02 | 3.51 | 2.42 | 3 |
| 288 | 2426988 | H94262 | — | | 1.16 | 3.60 | 3 | 1.70 | 2.11 | 3.21 | 4.15 | 4.88 | 1.97 | 3 |
| 289 | 364352 | AA022496 | bone | | 0.03 | 0.08 | 3 | 2.52 | 0.38 | 4.22 | 3.89 | 4.83 | 2.18 | 3 |
| 290 | 244060 | N34042 | — | | 0.06 | 0.17 | 3 | 1.60 | 3.79 | 1.68 | 2.22 | 3.29 | 5.43 | 3 |
| 291 | 544639 | AA074877 | skin | | 1.06 | 3.15 | 3 | 1.72 | 3.77 | 3.39 | 3.80 | 2.68 | 2.56 | 3 |
| 292 | 652520 | AA105051 | | | 17.95 | 53.64 | 3 | 3.39 | 3.07 | 2.81 | 4.99 | 2.83 | 0.82 | 3 |
| 293 | 797000 | AA463800 | t-Cell | | 0.11 | 0.33 | 3 | 1.54 | 3.21 | 1.65 | 5.62 | 1.07 | 4.77 | 3 |
| 294 | 1493252 | AA886870 | prostate | N/A | 18.18 | 54.06 | 3 | 4.82 | 3.25 | 2.81 | 3.26 | 1.52 | 2.20 | 3 |
| 295 | 447416 | AA702335 | brain | N/A | 1.15 | 3.41 | 3 | 5.58 | 4.11 | 2.12 | 1.04 | 3.10 | 1.84 | 3 |
| 296 | 1475306 | AA77997 | — | N/A | 1.54 | 4.58 | 3 | 2.92 | 3.27 | 4.63 | 1.90 | 1.93 | 3.19 | 3 |
| 297 | 194134 | H451039 | — | | 0.22 | 0.85 | 3 | 1.35 | 3.39 | 3.88 | 2.74 | 3.89 | 2.47 | 3 |
| 298 | 1486937 | AA863449 | muscle | | 1.15 | 3.38 | 3 | 5.57 | 3.38 | 1.71 | 2.34 | 0.80 | 3.84 | 3 |
| 299 | 201039 | H48278 | — | | 0.83 | 2.42 | 3 | 2.78 | 3.98 | 2.31 | 3.06 | 2.04 | 3.43 | 3 |
| 300 | 283769 | H99694 | — | | 0.75 | 2.19 | 3 | 3.82 | 7.88 | 3.41 | 1.10 | 1.06 | 0.29 | 3 |
| 301 | 427930 | AA001976 | — | | 0.05 | 0.14 | 3 | 0.99 | 3.14 | 8.49 | 0.21 | 4.44 | 2.22 | 3 |
| 302 | 307244 | N95187 | heart | | 0.35 | 1.01 | 3 | 2.20 | 4.92 | 3.17 | 2.07 | 3.85 | 1.25 | 3 |
| 303 | 810843 | AA456989 | brain | | 0.06 | 0.17 | 3 | 1.34 | 3.15 | 1.41 | 5.84 | 4.41 | 1.44 | 3 |
| 304 | 1460820 | AA889785 | bone | N/A | 3.62 | 10.49 | 3 | 2.35 | 2.11 | 3.29 | 1.57 | 3.43 | 4.83 | 3 |
| 305 | 26259 | R20547 | prostate | Y | 0.87 | 2.50 | 3 | 3.08 | 3.41 | 3.69 | 2.10 | 2.31 | 2.77 | 3 |
| 306 | 366154 | AA62813 | bone | | 0.01 | 0.03 | 3 | 3.87 | 3.77 | 1.00 | 1.23 | 1.00 | 6.23 | 3 |
| 307 | 282104 | N51498 | brain | | 0.93 | 2.68 | 3 | 2.68 | 4.75 | 3.79 | 1.52 | 0.39 | 3.94 | 3 |
| 308 | 344308 | W70147 | — | Y | 0.46 | 1.31 | 3 | 3.18 | 3.47 | 5.50 | 1.78 | 1.21 | 1.74 | 3 |
| 309 | 45525 | H08725 | prostate | Y | 2.19 | 8.17 | 3 | 2.20 | 3.24 | 3.16 | 3.52 | 2.33 | 2.40 | 3 |
| 310 | 196303 | R92435 | bone | | 0.04 | 0.11 | 3 | 2.58 | 4.53 | 3.04 | 2.48 | 3.30 | 0.05 | 3 |
| 311 | 267495 | N25262 | testis | Y | 1.59 | 4.40 | 3 | 3.88 | 3.24 | 2.59 | 3.41 | 1.52 | 2.19 | 3 |
| 312 | 757196 | AA496133 | | | 0.65 | 1.78 | 3 | 3.73 | 4.67 | 3.45 | 1.24 | 1.90 | 1.53 | 3 |
| 313 | 204122 | H61886 | — | | 1.31 | 3.59 | 3 | 2.38 | 4.23 | 3.40 | 3.07 | 2.93 | 0.52 | 3 |
| 314 | 839545 | AA489791 | — | | 0.42 | 1.14 | 3 | 5.37 | 0.99 | 1.38 | 1.18 | 3.64 | 3.82 | 3 |
| 315 | 809694 | AA454702 | brain | | 0.02 | 0.05 | 3 | 0.54 | 0.58 | 3.30 | 2.89 | 4.45 | 4.47 | 3 |
| 316 | 126234 | R06382 | — | | 0.30 | 0.81 | 3 | 2.13 | 3.70 | 2.31 | 1.13 | 3.56 | 3.32 | 3 |
| 317 | 280407 | N51563 | unspecified | N/A | 0.97 | 2.62 | 3 | 1.61 | 3.40 | 1.91 | 3.26 | 3.22 | 2.72 | 3 |
| 318 | 758284 | AA404231 | placenta | | 0.78 | 2.08 | 3 | 1.82 | 2.31 | 1.16 | 3.46 | 3.11 | 4.17 | 3 |
| 319 | 150314 | H00617 | t-cell | | 2.18 | 5.81 | 3 | 3.69 | 3.90 | 3.23 | 2.04 | 1.07 | 2.09 | 3 |
| 320 | 288936 | N82696 | — | | 0.04 | 0.11 | 3 | 1.59 | 3.47 | 3.00 | 0.99 | 2.99 | 3.97 | 3 |
| 321 | 757440 | AA437226 | | | 0.05 | 0.13 | 3 | 0.21 | 4.19 | 1.79 | 4.52 | 0.21 | 5.08 | 3 |
| 322 | 176554 | H45289 | — | N/A | 0.87 | 2.28 | 3 | 1.89 | 2.02 | 3.72 | 3.33 | 3.56 | 1.30 | 3 |
| 323 | 296057 | N69989 | heart | | 0.88 | 1.79 | 3 | 2.03 | 3.01 | 2.54 | 1.85 | 3.26 | 3.01 | 3 |
| 324 | 755299 | AA496359 | | | 1.69 | 4.39 | 3 | 4.47 | 0.52 | 1.93 | 3.99 | 0.70 | 3.96 | 3 |
| 325 | 725321 | AA291749 | breast | | 0.87 | 2.27 | 3 | 3.21 | 3.07 | 3.61 | 2.30 | 1.62 | 1.73 | 3 |
| 326 | 1467831 | AA883431 | — | N/A | 1.17 | 3.02 | 3 | 1.18 | 1.22 | 4.32 | 0.72 | 3.58 | 4.52 | 3 |
| 327 | 120097 | T95151 | Neuron | | 0.80 | 1.54 | 3 | 2.09 | 2.01 | 1.14 | 3.98 | 3.01 | 3.31 | 3 |
| 328 | 159453 | H15748 | brain | | 0.02 | 0.04 | 3 | 0.82 | 3.70 | 2.31 | 0.64 | 3.72 | 4.51 | 3 |
| 329 | 1460696 | AA868802 | — | N/A | 6.57 | 16.82 | 3 | 1.39 | 3.08 | 1.94 | 2.32 | 3.41 | 3.22 | 3 |
| 330 | 757366 | AA437126 | heart | | 0.02 | 0.06 | 3 | 3.92 | 2.20 | 3.37 | 0.77 | 0.24 | 4.83 | 3 |
| 331 | 450612 | AA682452 | — | N/A | 0.74 | 1.89 | 3 | 1.17 | 0.96 | 0.92 | 3.00 | 4.79 | 4.44 | 3 |
| 332 | 342543 | W88141 | bone | | 0.02 | 0.04 | 3 | 4.17 | 4.44 | 0.58 | 1.17 | 4.50 | 0.30 | 3 |
| 333 | 603741 | AA131486 | brain | | 1.05 | 2.85 | 3 | 3.31 | 3.85 | 3.58 | 1.37 | 1.19 1.85 | 3 | |
| 334 | 784130 | AA432075 | prostate | Maybe | 17.73 | 44.77 | 3 | 3.17 | 4.60 | 0.76 | 1.58 3.28 | 1.79 | 3 | |
| 335 | 325370 | W52208 | bone | | 0.03 | 0.08 | 3 | 3.68 | 3.87 | 2.92 | 1.13 | 0.31 | 3.14 | 3 |
| 336 | 288883 | N82304 | kidney | | 0.78 | 1.05 | 3 | 3.07 | 3.96 | 0.85 | 1.90 | 1.37 | 3.87 | 3 |
| 337 | 344359 | W73377 | lung | | 0.07 | 0.18 | 2 | 0.90 | 3.52 | 0.14 | 3.08 | 7.02 | 0.08 | 3 |
| 338 | 63301 | R18241 | — | | 1.15 | 2.79 | 2 | 3.60 | 3.57 | 3.34 | 1.40 | 1.23 | 1.35 | 3 |
| 339 | 420823 | AA000803 | — | | 0.14 | 0.35 | 2 | 3.16 | 3.19 | 1.82 | 1.85 | 1.08 | 3.65 | 3 |
| 340 | 258242 | N30655 | — | | 0.82 | 1.49 | 2 | 3.61 | 3.23 | 0.28 | 4.12 | 0.85 | 2.32 | 3 |
| 341 | 255807 | N27368 | — | | 0.00 | 0.20 | 2 | 0.60 | 4.21 | 1.00 | 1.52 | 3.40 | 3.14 | 3 |
| 342 | 452088 | AA707125 | testis | | 0.88 | 2.03 | 2 | 1.29 | 3.24 | 1.70 | 3.19 | 3.01 | 1.30 | 3 |
| 343 | 585779 | AA135870 | prostate | | 20.80 | 48.84 | 2 | 2.43 | 3.80 | 0.16 | 3.57 | 0.38 | 3.18 | 3 |
| 344 | 128506 | R10154 | prostate | | 0.04 | 0.00 | 2 | 1.20 | 3.87 | 0.24 | 3.30 | 0.24 | 4.05 | 3 |
| 345 | 839048 | AA487505 | brain | | 0.38 | 0.78 | 2 | 4.12 | 3.45 | 0.42 | 3.04 | 0.88 | 0.82 | 3 |
| 346 | 500853 | AA158162 | endothelial | | 0.01 | 0.18 | 13 | 0.74 | 0.74 | 1.96 | 0.74 | 42.48 | 32.88 | 2 |
| 347 | 60787 | H16633 | brain | | 0.08 | 0.50 | 8 | 1.60 | 0.16 | 2.38 | 2.67 | 34.78 | 5.82 | 2 |
| 348 | 137084 | R83085 | skin | | 0.29 | 2.25 | 8 | 8.43 | 33.82 | 0.37 | 0.58 | 1.11 | 2.44 | 2 |
| 349 | 866585 | AA660222 | breast | | 0.12 | 0.04 | 8 | 1.48 | 0.70 | 1.10 | 0.43 | 32.14 | 1.71 | 2 |
| 350 | 151477 | H02837 | skin | | 1.19 | 8.18 | 7 | 7.32 | 29.35 | 1.05 | 0.40 | 0.48 | 2.55 | 2 |
| 351 | 81331 | T60111 | | Y | 0.47 | 3.10 | 7 | 15.33 | 10.31 | 2.50 | 1.84 | 0.88 | 1.40 | 2 |
| 352 | 62026 | H22032 | — | | 0.25 | 1.66 | 7 | 13.67 | 22.88 | 0.57 | 1.63 | 1.01 | 0.38 | 2 |
| 353 | 850827 | AA886405 | colon | | 0.08 | 0.53 | 6 | 1.07 | 0.12 | 0.12 | 0.52 | 31.38 | 5.38 | 2 |
| 354 | 429088 | AA005106 | — | | 0.22 | 1.39 | 6 | 0.68 | 0.41 | 0.38 | 1.35 | 18.48 | 16.17 | 2 |
| 355 | 284449 | N21233 | — | | 0.06 | 0.32 | 5 | 6.08 | 2.35 | 1.00 | 0.55 | 1.30 | 20.80 | 2 |
| 356 | 754358 | AA38142 | bone | | 8.25 | 44.96 | 5 | 2.04 | 2.77 | 1.77 | 2.77 | 11.30 | 12.03 | 2 |
| 357 | 757185 | AA43950 | Neuron | | 0.03 | 0.18 | 6 | 0.47 | 1.51 | 0.04 | 20.25 | 2.08 | 5.30 | 2 |
| 358 | 585235 | AA138125 | breast | | 18.25 | 80.08 | 5 | 7.86 | 14.43 | 2.52 | 1.47 | 0.61 | 2.40 | 2 |
| 359 | 47355 | H410081 | | | 0.10 | 0.48 | 5 | 14.30 | 0.10 | 0.55 | 0.22 | 0.05 | 2.80 | 2 |
| 360 | 377898 | AA777008 | testis | N/A | 3.49 | 15.28 | 4 | 5.72 | 2.44 | 11.88 | 1.20 | 2.30 | 2.82 | 2 |
| 361 | 745072 | AA26275 | | N/A | 0.45 | 1.95 | 4 | 6.41 | 14.60 | 1.23 | 1.41 | 1.47 | 0.03 | 2 |
| 362 | 79763 | T83981 | lung | | 0.18 | 0.71 | 4 | 1.17 | 7.49 | 1.07 | 1.48 | 0.08 | 14.84 | 2 |
| 363 | 1380018 | AA855158 | colon | | 0.05 | 0.20 | 4 | 11.10 | 10.66 | 0.60 | 0.52 | 0.21 | 1.48 | 2 |
| 364 | 283335 | N61441 | — | | 0.18 | 0.72 | 4 | 1.22 | 10.53 | 5.88 | 2.87 | 1.88 | 1.99 | 2 |
| 365 | 284931 | N71861 | — | | 0.02 | 0.07 | 4 | 2.68 | 8.38 | 0.83 | 0.57 | 0.02 | 10.57 | 2 |
| 366 | 1031203 | AA800082 | — | | 0.03 | 0.11 | 4 | 1.17 | 0.38 | 5.13 | 1.84 | 0.38 | 15.08 | 2 |
| 367 | 1048008 | AA778638 | endothelial | N/A | 0.14 | 0.57 | 4 | 10.73 | 5.06 | 2.06 | 2.22 | 1.03 | 0.58 | 2 |
| 368 | 481890 | AA682274 | breast | N/A | 10.27 | 38.51 | 4 | 0.81 | 2.56 | 2.08 | 8.45 | 6.08 | 1.84 | 2 |
| 369 | 50802 | H17628 | adrenal | | 0.31 | 1.18 | 4 | 1.87 | 2.07 | 2.88 | 2.21 | 5.85 | 8.87 | 2 |
| 370 | 382926 | AA018980 | brain | | 1.07 | 4.00 | 4 | 2.76 | 5.27 | 1.60 | 11.37 | 0.71 | 0.71 | 2 |
| 371 | 122178 | T98815 | — | | 0.03 | 0.11 | 4 | 0.65 | 8.21 | 0.34 | 1.94 | 0.20 | 10.81 | 2 |
| 372 | 248806 | N53214 | b | | 0.58 | 2.08 | 4 | 6.45 | 12.29 | 1.20 | 0.84 | 0.62 | 0.85 | 2 |
| 373 | 101686 | H40350 | — | | 0.81 | 2.24 | 4 | 2.33 | 6.76 | 2.63 | 2.08 | 5.39 | 1.00 | 2 |
| 374 | 155072 | R71393 | — | | 0.30 | 1.07 | 4 | 7.00 | 0.56 | 2.16 | 0.02 | 0.72 | 1.33 | 2 |
| 375 | 802438 | AA134882 | bone | | 1.10 | 3.04 | 4 | 1.28 | 2.12 | 2.17 | 7.62 | 1.52 | 8.78 | 2 |
| 376 | 293889 | N8975 | breast | | 0.01 | 0.03 | 4 | 0.95 | 1.08 | 1.08 | 1.03 | 10.16 | 1.08 | 2 |
| 377 | 510138 | AA083239 | muscle | | 0.03 | 0.10 | 4 | 8.17 | 0.37 | 7.88 | 1.98 | 1.85 | 2.87 | 2 |
| 378 | 878264 | A870280 | — | N/A | 2.85 | 0.63 | 3 | 1.42 | 1.18 | 2.71 | 7.16 | 5.68 | 2.39 | 2 |
| 379 | 289025 | N59626 | breast | | 0.85 | 1.62 | 3 | 2.20 | 8.29 | 1.81 | 6.02 | 1.09 | 1.88 | 2 |
| 380 | 84713 | T74257 | — | | 0.07 | 0.21 | 3 | 8.85 | 8.18 | 2.97 | 2.70 | 0.15 | 0.89 | 2 |
| 381 | 796648 | AA80115 | lung | | 9.12 | 29.44 | 3 | 1.38 | 0.93 | 1.89 | 8.52 | 1.58 | 5.09 | 2 |
| 382 | 49315 | H15386 | — | | 0.22 | 0.71 | 3 | 0.89 | 1.60 | 1.44 | 5.85 | 7.72 | 2.25 | 2 |
| 383 | 1684274 | AA4003028 | — | N/A | 1.45 | 4.54 | 3 | 5.71 | 7.10 | 2.75 | 1.83 | 0.65 | 1.06 | 2 |
| 384 | 384555 | AA22601 | — | | 1.64 | 5.67 | 3 | 5.38 | 2.73 | 1.58 | 1.33 | 5.68 | 1.60 | 2 |
| 385 | 743888 | AA629324 | testis | N/A | 1.27 | 3.90 | 3 | 2.80 | 2.21 | 5.28 | 5.17 | 1.89 | 1.28 | 2 |
| 386 | 50114 | H16743 | brain | | 2.07 | 5.53 | 3 | 2.59 | 6.02 | 0.45 | 5.94 | 0.35 | 0.70 | 2 |
| 367 | 342184 | W83776 | — | | 0.04 | 0.10 | 3 | 0.80 | 0.28 | 5.08 | 1.19 | 0.26 | 6.48 | 2 |
| 388 | 795407 | AA453516 | lung | | 0.13 | 0.32 | 2 | 0.33 | 0.97 | 1.47 | 5.45 | 0.62 | 5.62 | 2 |
| 389 | 110503 | T89998 | prostate | | 0.60 | 1.45 | 2 | 0.73 | 1.24 | 0.76 | 0.92 | 5.55 | 5.38 | 2 |

TABLE 2-3
T3NO vs. BPH: Gene upregulated 10 fold or higher in at
least 2 tumor samples or 5 fold or higher in at least 3 tumor samples.

| | | Gen Bank | | Secreted/ | | | Fold/ | Fold | Fold | Fold | Fold | Fold | Fold | # of |
|---|---|---|---|---|---|---|---|---|---|---|---|---|---|---|
| | IMAGE | Accession | Tissue | Membrane | | | (Ave Tumor/A | (MPM 120/A | (MPM 121/A | (MPM 124/A | (MPM 122/A | (MPM 123/A | (MPM 125/A | Tumors |
| Order | Clone ID | Number | Distribution | Bound | AveBPH | AveTumor | vsBPH) | vsBPH) | vsBPH) | vsBPH) | vsBPh) | vsBPH) | vsBPH) | Samples |
| 1 | 788180 | AA483310 | prostate | | 1.98 | 133.47 | 87.99 | 149.83 | 165.70 | 7.33 | 62.32 | 22.54 | 10.38 | 6 |
| 2 | 048037 | AA196979 | prostate | | 2.38 | 114.00 | 47.98 | 65.24 | 24.62 | 77.47 | 31.08 | 63.59 | 35.00 | 6 |
| 3 | 284701 | N64840 | prostate | Y | 2.41 | 73.10 | 30.37 | 31.27 | 50.07 | 34.87 | 5.28 | 41.24 | 19.50 | 8 |
| 4 | 450049 | AA703398 | prostate | N/A | 2.55 | 76.64 | 30.08 | 38.52 | 11.57 | 48.94 | 15.57 | 41.42 | 20.45 | 6 |
| 5 | 248788 | N55268 | — | | 0.01 | 0.21 | 22.49 | 11.34 | 8.80 | 10.09 | 19.72 | 21.41 | 63.61 | 6 |
| 6 | 281003 | N50880 | t-cell | | 24.08 | 423.85 | 17.62 | 22.19 | 20.64 | 22.40 | 16.57 | 11.61 | 13.29 | 6 |
| 7 | 138806 | R35051 | | Y | 2.56 | 44.38 | 17.34 | 29.78 | 41.52 | 9.28 | 10.57 | 5.71 | 7.21 | 6 |
| 8 | 133130 | R28397 | prostate | Y | 1.48 | 25.48 | 17.23 | 30.48 | 41.34 | 6.15 | 9.24 | 10.23 | 5.98 | 6 |
| 9 | 179753 | H51549 | bone | | 0.01 | 0.12 | 11.81 | 9.28 | 11.64 | 11.99 | 10.01 | 17.09 | 10.88 | 6 |
| 10 | 51433 | H20747 | — | | 2.07 | 19.58 | 9.43 | 6.78 | 8.32 | 8.58 | 7.12 | 8.55 | 19.24 | 8 |
| 11 | 106975 | R91398 | — | | 0.50 | 3.83 | 7.87 | 5.89 | 6.80 | 8.91 | 5.51 | 12.71 | 6.41 | 6 |
| 12 | 287528 | N82098 | — | | 0.30 | 3.88 | 12.94 | 15.20 | 2.45 | 15.94 | 12.53 | 19.49 | 9.01 | 5 |
| 13 | 259374 | N31952 | prostate | | 1.20 | 13.52 | 10.47 | 7.91 | 24.39 | 10.24 | 5.27 | 13.26 | 1.75 | 5 |
| 14 | 1030855 | AA821761 | — | Y | 1.24 | 12.57 | 10.16 | 7.99 | 15.81 | 11.93 | 6.14 | 4.27 | 15.03 | 5 |
| 15 | 1031599 | AA809485 | — | | 0.27 | 2.07 | 7.65 | 5.89 | 10.98 | 8.72 | 12.64 | 2.63 | 7.06 | 5 |
| 16 | 21931 | T72543 | breast | | 0.29 | 1.97 | 6.87 | 3.24 | 10.05 | 8.80 | 7.18 | 5.75 | 8.20 | 5 |
| 17 | 825859 | AA504784 | — | N/A | 1.91 | 12.19 | 8.37 | 8.28 | 4.64 | 5.38 | 5.13 | 5.23 | 11.57 | 8 |
| 18 | 130138 | R20813 | — | | 0.04 | 0.20 | 5.82 | 7.23 | 8.80 | 5.30 | 5.38 | 8.98 | 2.08 | 5 |
| 19 | 1488280 | AA884926 | — | N/A | 0.65 | 22.38 | 34.18 | 18.89 | 1.69 | 59.34 | 105.24 | 0.80 | 19.00 | 4 |
| 20 | 743828 | AA834379 | bone | N/A | 0.58 | 12.90 | 22.20 | 29.78 | 48.11 | 29.58 | 0.88 | 23.62 | 1.28 | 4 |
| 21 | 41647 | R52794 | brain | | 0.40 | 8.34 | 15.84 | 9.85 | 9.81 | 1.44 | 57.03 | 15.40 | 1.72 | 4 |
| 22 | 764168 | AA432103 | prostate | | 15.97 | 194.72 | 12.19 | 9.82 | 26.16 | 14.54 | 17.70 | 1.22 | 3.92 | 4 |
| 23 | 193923 | R83407 | brain | Y | 17.59 | 158.73 | 9.02 | 10.87 | 18.00 | 7.67 | 13.10 | 1.22 | 3.27 | 4 |
| 24 | 487165 | AA045074 | colon | | 0.01 | 0.09 | 8.98 | 13.49 | 10.45 | 13.83 | 13.97 | 1.00 | 1.00 | 4 |
| 25 | 308066 | N91003 | | Y | 0.80 | 6.94 | 8.70 | 13.54 | 16.10 | 7.85 | 4.72 | 3.85 | 8.31 | 4 |
| 26 | 292217 | N79180 | bone | | 0.08 | 0.42 | 8.73 | 3.94 | 10.11 | 7.14 | 10.04 | 4.07 | 5.11 | 4 |
| 27 | 144740 | R78229 | prostate | | 0.19 | 1.27 | 8.55 | 7.58 | 10.38 | 6.62 | 10.48 | 1.64 | 2.63 | 4 |
| 28 | 482488 | AA899895 | — | N/A | 0.38 | 2.39 | 6.34 | 5.95 | 10.82 | 5.48 | 10.02 | 1.94 | 3.81 | 4 |
| 29 | 1058172 | AA820995 | muscle | | 11.81 | 70.00 | 8.03 | 0.88 | 5.08 | 10.11 | 2.19 | 1.74 | 10.42 | 4 |
| 30 | 782383 | AA431407 | — | | 0.95 | 5.55 | 5.84 | 8.95 | 11.77 | 5.12 | 5.02 | 1.51 | 2.87 | 4 |
| 31 | 195034 | R88764 | | | 0.02 | 0.14 | 5.84 | 3.19 | 6.01 | 6.21 | 7.48 | 3.87 | 6.48 | 4 |
| 32 | 481541 | AA705258 | — | N/A | 0.34 | 1.88 | 5.48 | 2.80 | 6.47 | 9.51 | 6.43 | 5.07 | 2.77 | 4 |
| 33 | 787938 | AA452278 | prostate | Y | 1.78 | 8.87 | 5.04 | 6.20 | 9.64 | 6.65 | 1.30 | 0.28 | 5.98 | 4 |
| 34 | 754034 | AA479058 | — | | 0.02 | 0.10 | 5.01 | 5.74 | 8.87 | 0.33 | 1.52 | 8.64 | 9.17 | 4 |
| 35 | 195387 | R88915 | — | | 0.47 | 2.07 | 4.38 | 5.34 | 8.45 | 8.39 | 1.35 | 5.02 | 1.71 | 4 |
| 36 | 638888 | AA457235 | prostate | | 3.44 | 48.10 | 13.39 | 11.55 | 50.28 | 3.85 | 4.31 | 3.32 | 7.01 | 3 |
| 37 | 281039 | N47717 | endothelial | | 0.78 | 9.99 | 12.85 | 43.16 | 25.22 | 5.01 | 2.22 | 0.30 | 1.22 | 3 |
| 38 | 490718 | AA115761 | embryo | | 0.04 | 0.48 | 12.37 | 48.34 | 8.87 | 1.83 | 0.27 | 18.31 | 0.82 | 3 |
| 39 | 744044 | AA825890 | prostate | | 1.90 | 22.42 | 11.81 | 2.78 | 4.13 | 1.88 | 8.52 | 48.21 | 5.35 | 3 |
| 40 | 287726 | N59158 | — | Y | 0.51 | 6.43 | 10.69 | 20.89 | 31.93 | 7.51 | 1.25 | 0.88 | 1.67 | 3 |
| 41 | 1034473 | AA779726 | prostate | N/A | 0.13 | 1.38 | 10.50 | 22.49 | 23.74 | 3.29 | 9.26 | 2.32 | 1.91 | 3 |
| 42 | 1456424 | AA862902 | breast | | 0.08 | 0.89 | 8.20 | 10.16 | 21.53 | 1.58 | 831 | 4.64 | 2.76 | 3 |
| 43 | 380155 | AA013260 | | | 0.01 | 0.11 | 8.02 | 10.07 | 0.73 | 2.20 | 24.39 | 4.57 | 6.15 | 3 |
| 44 | 80100 | T72330 | bone | | 1.71 | 11.25 | 6.59 | 7.39 | 5.18 | 4.03 | 15.83 | 2.08 | 4.49 | 3 |
| 45 | 268211 | N29986 | prostate | | 0.57 | 3.71 | 8.48 | 6.59 | 3.88 | 13.77 | 2.12 | 4.38 | 6.18 | 3 |
| 46 | 593186 | AA165078 | | Y | 9.70 | 60.24 | 8.21 | 9.37 | 12.99 | 2.44 | 0.80 | 2.35 | 3.29 | 3 |
| 47 | 426810 | AA004081 | — | | 0.10 | 0.80 | 6.03 | 6.54 | 8.44 | 8.60 | 4.79 | 4.07 | 3.73 | 3 |
| 48 | 611582 | AA454597 | bone | Y | 3.59 | 21.22 | 5.92 | 9.12 | 4.48 | 4.71 | 6.04 | 2.69 | 8.45 | 3 |
| 49 | 426430 | AA004412 | — | | 0.53 | 3.12 | 5.83 | 1.13 | 4.85 | 4.73 | 5.71 | 11.11 | 7.88 | 3 |
| 50 | 795720 | AA400291 | unspecified | | 0.02 | 0.11 | 5.58 | 5.69 | 4.90 | 0.83 | 11.93 | 6.34 | 3.73 | 3 |
| 51 | 712950 | AA262273 | t-cell | | 5.93 | 32.98 | 5.50 | 8.81 | 14.24 | 8.62 | 0.72 | 0.88 | 2.42 | 3 |
| 52 | 825785 | AA505117 | ovuay | | 1.50 | 8.57 | 5.51 | 3.24 | 8.98 | 2.95 | 0.31 | 9.01 | 4.56 | 3 |
| 53 | 804761 | AA148735 | bone | | 0.08 | 0.43 | 5.45 | 6.26 | 0.51 | 7.72 | 0.13 17.97 | 0.10 | 3 | |
| 54 | 491706 | AA118259 | breast | | 0.75 | 3.90 | 5.23 | 3.93 | 6.17 | 7.31 | 9.02 | 2.29 | 2.07 | 3 |
| 55 | 241821 | H93381 | — | N/A | 1.06 | 5.43 | 5.10 | 3.95 | 8.61 | 5.92 | 8.06 | 2.79 | 3.31 | 3 |
| 56 | 782537 | AA448484 | testis | | 1.72 | 8.80 | 5.01 | 2.52 | 2.76 | 8.08 | 0.99 | 7.48 | 8.28 | 3 |
| 57 | 277596 | N57002 | bone | Y | 1.48 | 7.34 | 4.97 | 7.68 | 12.12 | 2.17 | 0.90 | 1.13 | 5.64 | 3 |
| 58 | 124128 | R01348 | — | Y | 1.25 | 6.16 | 4.92 | 2.53 | 5.09 | 3.89 | 4.46 | 8.46 | 5.11 | 3 |
| 59 | 134719 | R28287 | colon | | 0.29 | 1.40 | 4.85 | 5.02 | 7.58 | 4.08 | 5.94 | 3.53 | 2.93 | 3 |
| 60 | 614306 | AA459318 | prostate | | 9.85 | 45.42 | 4.71 | 5.23 | 10.10 | 4.41 | 5.26 | 1.16 | 2.07 | 3 |
| 61 | 781233 | AA440316 | — | | 0.02 | 0.10 | 4.70 | 0.48 | 4.83 | 6.38 | 7.51 | 1.30 | 7.69 | 3 |
| 62 | 34888 | R10878 | — | | 0.14 | 0.67 | 4.62 | 5.09 | 9.05 | 6.74 | 1.93 | 1.68 | 2.85 | 3 |
| 63 | 280170 | N32072 | prostate | | 12.86 | 59.29 | 4.61 | 5.52 | 5.50 | 9.33 | 1.01 | 1.63 | 4.68 | 3 |
| 64 | 277327 | N57483 | brain | | 0.02 | 0.09 | 4.56 | 1.67 | 6.21 | 2.55 | 8.18 | 1.05 | 7.68 | 3 |
| 65 | 307249 | N93436 | testis | | 3.36 | 15.26 | 4.54 | 9.25 | 5.31 | 0.40 | 5.39 | 3.29 | 3.63 | 3 |
| 66 | 770066 | AA430545 | brain | | 7.38 | 33.38 | 4.52 | 7.28 | 8.45 | 8.19 | 0.54 | 0.26 | 2.38 3 | |
| 67 | 51620 | H19448 | — | | 0.03 | 0.12 | 4.49 | 1.90 | 8.33 | 7.29 | 6.63 | 2.43 | 0.38 | 3 |
| 68 | 428267 | AA004028 | unspecified | | 1.41 | 8.20 | 4.38 | 2.17 | 5.43 | 7.71 | 1.35 | 3.96 | 5.85 | 3 |
| 69 | 276504 | N68139 | brain | | 0.06 | 0.22 | 4.34 | 0.98 | 7.98 | 1.80 | 5.23 | 1.24 | 8.83 | 3 |
| 70 | 205828 | H57585 | prostate | | 0.51 | 2.18 | 4.28 | 7.89 | 2.40 | 5.57 | 1.28 | 1.92 | 6.79 | 3 |
| 71 | 285005 | N21061 | skin | | 0.08 | 0.20 | 4.23 | 8.18 | 3.28 | 3.22 | 6.00 | 0.56 | 6.16 | 3 |
| 72 | 67397 | T49325 | testis | | 0.01 | 0.06 | 4.16 | 5.29 | 7.99 | 0.68 | 0.90 | 0.68 | 9.45 | 3 |
| 73 | 264624 | N04790 | — | | 1.58 | 6.36 | 4.03 | 2.09 | 6.18 | 8.48 | 2.24 | 6.89 | 1.52 | S |
| 74 | 862497 | AA878804 | bone | | 0.04 | 0.14 | 3.94 | 0.28 | 7.83 | 0.28 | 9.35 | 0.79 | 5.34 | 3 |
| 75 | 856174 | AA630620 | | | 0.03 | 0.11 | 3.89 | 8.41 | 8.16 | 0.98 | 0.38 | 1.33 | 8.10 | 3 |
| 76 | 503624 | AA133959 | brain | | 0.12 | 0.44 | 3.78 | 1.82 | 0.09 | 1.76 | 6.89 | 6.74 | 6.37 | 3 |
| 77 | 206210 | H65288 | placenta | | 0.89 | 3.34 | 3.76 | 5.28 | 5.92 | 7.33 | 1.89 | 0.76 | 1.61 | 3 |
| 78 | 325380 | AA264285 | spleen | | 0.01 | 0.03 | 3.40 | 1.86 | 1.13 | 5.78 | 5.41 | 1.13 | 5.09 | 3 |
| 79 | 270917 | N32514 | | | 0.01 | 0.23 | 25.92 | 3.34 | 1.13 | 1.13 | 14.34 | 134.44 | 1.13 | 2 |
| 80 | 1390584 | AA643592 | prostate | | 1.64 | 33.09 | 18.02 | 49.03 | 48.43 | 3.22 | 1.15 | 2.77 | 3.51 | 2 |
| 81 | 782233 | A431721 | prostate | Y | 0.20 | 3.10 | 15.42 | 64.92 | 1.22 | 0.11 | 0.61 | 3.63 | 22.02 | 2 |
| 82 | 293579 | N94111 | spleen | | 0.96 | 12.90 | 13.42 | 0.73 | 1.52 | 4.77 | 26.47 | 44.95 | 2.08 | 2 |
| 83 | 890883 | AA156162 | endothelial | | 0.01 | 0.18 | 13.25 | 0.74 | 0.74 | 1.90 | 0.74 | 42.44 | 32.88 | 2 |
| 84 | 282080 | N48259 | prostate | Maybe | 1.90 | 19.34 | 10.17 | 21.89 | 29.40 | 4.51 | 1.88 | 1.07 | 2.28 | 2 |
| 85 | 731200 | AA410627 | breast | | 4.08 | 33.97 | 8.32 | 14.84 | 28.90 | 1.81 | 1.28 | 0.78 | 4.21 | 2 |
| 86 | 81331 | T50111 | | | 0.47 | 3.19 | 8.81 | 15.33 | 19.31 | 2.50 | 1.84 | 0.88 | 1.40 | 2 |
| 87 | 52026 | H22932 | — | | 0.25 | 1.68 | 6.69 | 13.67 | 22.88 | 0.57 | 1.63 | 1.01 | 0.36 | 2 |
| 88 | 24915 | R39088 | — | | 0.93 | 5.97 | 6.42 | 3.07 | 2.31 | 4.37 | 2.85 | 10.25 | 15.87 | 2 |
| 89 | 298417 | N74131 | colon | | 0.08 | 0.48 | 8.33 | 13.15 | 16.83 | 1.72 | 1.58 | 1.47 | 3.24 | 2 |
| 90 | 429088 | AA005196 | — | | 0.22 | 1.39 | 6.27 | 0.88 | 0.41 | 0.38 | 1.35 | 18.40 | 18.17 | 2 |
| 91 | 754358 | AA436142 | bone | | 8.25 | 44.96 | 5.45 | 2.04 | 2.77 | 1.77 | 2.77 | 11.30 | 12.03 | 2 |
| 92 | 1389018 | AA855158 | colon | | 0.05 | 0.20 | 4.11 | 11.10 | 10.88 | 0.50 | 0.52 | 0.21 | 1.40 | 2 |

TABLE 2-4
Poor vs Good Clinical Outcome: Genes
upregulated 3 fold or higher in any one tumor sample.

| | | | | | Fold | Fold | Fold | Fold | Fold | # of |
|---|---|---|---|---|---|---|---|---|---|---|
| | IMAGE | Accession | | | (AvePoor/ | (mpm 31/ | (mpm 32/ | (mpm 33/ | (mpm 34/ | Tumors |
| Order | Clone ID | No. | AveGood | AvePoor | AveGood) | AveGood) | AveGood) | AveGood) | AveGood) | Samples |
| 1 | 753076 | AA436479 | 1.42 | 42.16 | 29.71 | 2.52 | 57.56 | 34.98 | 23.80 | 3 |
| 2 | 124071 | R02686 | 0.53 | 3.21 | 6.08 | 0.69 | 16.64 | 3.34 | 3.64 | 3 |
| 3 | 346321 | W79647 | 0.64 | 3.29 | 5.17 | 3.69 | 7.75 | 1.51 | 7.73 | 3 |
| 4 | 855624 | AA664101 | 24.18 | 108.55 | 4.49 | 4.12 | 6.82 | 0.22 | 6.80 | 3 |
| 5 | 42325 | R61073 | 13.03 | 52.25 | 4.01 | 4.90 | 5.91 | 4.66 | 0.57 | 3 |
| 6 | 287528 | | 2.13 | 6.32 | 2.97 | 4.00 | 3.29 | 1.30 | 3.29 | 3 |
| 7 | 756595 | AA444051 | 1.46 | 3.89 | 2.67 | 0.45 | 3.61 | 3.01 | 3.60 | 3 |
| 8 | 452374 | AA700876 | 0.64 | 19.98 | 31.26 | 0.24 | 62.30 | 0.37 | 62.14 | 2 |
| 9 | 357105 | W93315 | 0.87 | 8.54 | 9.81 | 0.81 | 25.33 | 12.26 | 0.86 | 2 |
| 10 | 460798 | AA708152 | 1.24 | 6.95 | 5.61 | 0.47 | 17.80 | 3.83 | 0.33 | 2 |
| 11 | 284601 | N76201 | 0.27 | 1.50 | 5.49 | 0.82 | 10.19 | 0.77 | 10.17 | 2 |
| 12 | 460487 | AA677706 | 53.51 | 237.74 | 4.44 | 0.02 | 8.87 | 0.03 | 8.85 | 2 |
| 13 | 248412 | N58558 | 0.86 | 3.63 | 4.25 | 0.95 | 7.66 | 0.75 | 7.64 | 2 |
| 14 | 212188 | H68848 | 0.58 | 2.37 | 4.23 | 0.93 | 6.76 | 2.47 | 6.75 | 2 |
| 15 | 782513 | AA432030 | 4.08 | 16.09 | 3.95 | 1.06 | 1.58 | 6.19 | 6.98 | 2 |
| 16 | 782513 | AA432030 | 2.03 | 7.78 | 3.83 | 1.37 | 1.26 | 6.00 | 6.70 | 2 |
| 17 | 129128 | R10935 | 0.38 | 1.47 | 3.81 | 0.92 | 3.71 | 0.70 | 9.92 | 2 |
| 18 | 854678 | AA630084 | 6.04 | 21.23 | 3.52 | 1.40 | 4.21 | 2.50 | 5.95 | 2 |
| 19 | 362059 | AA001431 | 1.47 | 5.16 | 3.51 | 1.66 | 5.87 | 0.66 | 5.88 | 2 |
| 20 | 454822 | AA677388 | 0.60 | 2.09 | 3.47 | 0.37 | 6.64 | 0.26 | 6.62 | 2 |
| 21 | 308896 | N92842 | 0.05 | 0.16 | 3.28 | 1.59 | 6.55 | 0.00 | 4.97 | 2 |
| 22 | 415766 | W84751 | 1.20 | 3.83 | 3.19 | 0.75 | 5.82 | 0.36 | 5.81 | 2 |
| 23 | 489535 | AA099262 | 3.91 | 12.41 | 3.17 | 5.95 | 0.84 | 1.37 | 4.54 | 2 |
| 24 | 384665 | AA028171 | 0.80 | 2.53 | 3.17 | 3.39 | 2.18 | 4.93 | 2.18 | 2 |
| 25 | 740941 | AA478298 | 53.79 | 169.35 | 3.15 | 1.55 | 5.32 | 0.41 | 5.31 | 2 |
| 26 | 241705 | H91680 | 1.71 | 5.19 | 3.04 | 1.25 | 4.71 | 5.51 | 0.69 | 2 |
| 27 | 754559 | AA406301 | 1.32 | 3.96 | 3.00 | 2.54 | 3.88 | 4.35 | 1.25 | 2 |
| 28 | 487327 | AA045524 | 0.49 | 1.46 | 2.96 | 1.51 | 4.72 | 0.91 | 4.72 | 2 |
| 29 | 843098 | AA488676 | 5.29 | 15.54 | 2.94 | 1.27 | 2.66 | 3.34 | 4.49 | 2 |
| 30 | 298303 | N73958 | 0.22 | 0.65 | 2.94 | 0.99 | 4.79 | 1.18 | 4.79 | 2 |
| 31 | 815542 | AA456886 | 1.41 | 4.08 | 2.90 | 1.32 | 1.80 | 4.07 | 4.42 | 2 |
| 32 | 782537 | AA432049 | 1.48 | 4.26 | 2.87 | 0.83 | 0.86 | 4.85 | 4.96 | 2 |
| 33 | 324437 | W46900 | 1.10 | 3.16 | 2.86 | 0.62 | 5.28 | 0.29 | 5.26 | 2 |
| 34 | 461759 | AA682386 | 0.67 | 1.86 | 2.79 | 1.13 | 4.59 | 0.83 | 4.59 | 2 |
| 35 | 754393 | AA436317 | 0.01 | 0.02 | 2.67 | 5.43 | 3.14 | 2.12 | 0.00 | 2 |
| 36 | 770983 | AA428415 | 16.45 | 43.42 | 2.64 | 1.19 | 3.67 | 2.04 | 3.66 | 2 |
| 37 | 415692 | W84716 | 0.65 | 1.70 | 2.60 | 1.72 | 3.82 | 1.06 | 3.81 | 2 |
| 38 | 435820 | AA701519 | 1.20 | 3.12 | 2.59 | 3.20 | 1.28 | 3.41 | 2.47 | 2 |
| 39 | 823896 | AA489743 | 1.17 | 3.00 | 2.56 | 0.92 | 1.43 | 3.94 | 3.92 | 2 |
| 40 | 811000 | AA485508 | 7.79 | 19.45 | 2.50 | 0.66 | 4.20 | 0.94 | 4.19 | 2 |
| 41 | 754600 | AA411384 | 6.54 | 15.91 | 2.43 | 1.99 | 1.25 | 3.06 | 3.45 | 2 |
| 42 | 41214 | R56898 | 0.08 | 0.19 | 2.43 | 1.43 | 3.22 | 1.85 | 3.21 | 2 |
| 43 | 898096 | AA598795 | 3.43 | 8.05 | 2.35 | 1.11 | 1.22 | 3.21 | 3.86 | 2 |
| 44 | 1473304 | AA916325 | 3.39 | 7.96 | 2.35 | 1.13 | 4.30 | 3.08 | 0.87 | 2 |
| 45 | 359835 | AA011215 | 29.86 | 69.88 | 2.34 | 3.49 | 3.72 | 0.91 | 1.24 | 2 |
| 46 | 430231 | AA010221 | 0.55 | 1.28 | 2.34 | 1.13 | 3.86 | 0.53 | 3.83 | 2 |
| 47 | 376516 | AA041499 | 2.44 | 5.70 | 2.34 | 1.48 | 1.07 | 3.21 | 3.59 | 2 |
| 48 | 323238 | W42812 | 2.09 | 4.88 | 2.34 | 0.57 | 4.18 | 0.41 | 4.17 | 2 |
| 49 | 770880 | AA434502 | 20.98 | 48.48 | 2.31 | 0.42 | 3.59 | 1.65 | 3.58 | 2 |
| 50 | 303099 | W20458 | 1.81 | 4.15 | 2.29 | 1.40 | 3.03 | 1.72 | 3.02 | 2 |
| 51 | 39959 | R53558 | 0.06 | 0.13 | 2.21 | 1.76 | 3.34 | 0.34 | 3.39 | 2 |
| 52 | 138788 | R63646 | 0.49 | 1.05 | 2.16 | 0.94 | 3.49 | 0.72 | 3.49 | 2 |
| 53 | 52071 | H24350 | 0.21 | 0.44 | 2.15 | 1.28 | 3.16 | 0.97 | 3.19 | 2 |
| 54 | 47418 | H11063 | 2.05 | 4.37 | 2.13 | 0.39 | 3.33 | 1.47 | 3.32 | 2 |
| 55 | 787938 | AA452278 | 2.20 | 4.63 | 2.10 | 0.89 | 0.69 | 3.21 | 3.63 | 2 |
| 56 | 796806 | AA461174 | 0.45 | 0.92 | 2.06 | 1.38 | 3.16 | 0.53 | 3.16 | 2 |
| 57 | 223350 | H86554 | 0.70 | 1.35 | 1.93 | 0.44 | 3.45 | 0.36 | 3.44 | 2 |
| 58 | 510136 | AA053884 | 0.05 | 0.09 | 1.88 | 0.16 | 3.22 | 0.95 | 3.20 | 2 |
| 59 | 741977 | AA401441 | 4.15 | 7.71 | 1.86 | 0.53 | 3.20 | 0.51 | 3.19 | 2 |
| 60 | 812074 | AA455988 | 0.74 | 6.43 | 8.67 | 0.85 | 0.65 | 31.70 | 1.47 | 1 |
| 61 | 305809 | N90051 | 0.56 | 3.02 | 5.39 | 16.87 | 1.63 | 1.44 | 1.63 | 1 |
| 62 | 282104 | N51498 | 1.64 | 7.68 | 4.69 | 0.58 | 0.71 | 2.08 | 15.38 | 1 |
| 63 | 795809 | AA460511 | 3.68 | 17.08 | 4.65 | 16.11 | 1.02 | 0.64 | 0.82 | 1 |
| 64 | 161456 | H25546 | 1.53 | 7.06 | 4.63 | 0.46 | 17.43 | 0.30 | 0.31 | 1 |
| 65 | 857442 | AA782380 | 0.18 | 0.82 | 4.45 | 2.95 | 1.61 | 1.16 | 12.08 | 1 |
| 66 | 1455740 | AA863204 | 0.09 | 0.39 | 4.41 | 15.34 | 0.92 | 0.97 | 0.42 | 1 |
| 67 | 141336 | R63823 | 0.82 | 3.58 | 4.37 | 1.91 | 0.70 | 13.86 | 1.00 | 1 |
| 68 | 450948 | AA704718 | 0.49 | 2.10 | 4.25 | 0.51 | 1.15 | 0.84 | 14.50 | 1 |
| 69 | 343919 | W69960 | 1.16 | 4.89 | 4.23 | 1.38 | 0.94 | 1.26 | 13.33 | 1 |
| 70 | 192258 | H41222 | 1.16 | 4.59 | 3.97 | 0.67 | 0.90 | 0.83 | 13.47 | 1 |
| 71 | 50930 | H19128 | 1.66 | 6.59 | 3.97 | 2.12 | 0.52 | 12.60 | 0.62 | 1 |
| 72 | 1032774 | AA628410 | 0.66 | 2.51 | 3.80 | 0.84 | 10.78 | 2.26 | 1.33 | 1 |
| 73 | 282117 | N46261 | 0.81 | 3.06 | 3.78 | 1.08 | 1.21 | 0.90 | 11.93 | 1 |
| 74 | 773157 | AA425382 | 0.86 | 3.20 | 3.74 | 0.62 | 1.09 | 12.18 | 1.09 | 1 |
| 75 | 320857 | W38679 | 1.57 | 5.85 | 3.73 | 11.18 | 1.48 | 0.80 | 1.48 | 1 |
| 76 | 786657 | AA451886 | 4.95 | 18.28 | 3.69 | 0.43 | 0.31 | 0.40 | 13.64 | 1 |
| 77 | 44351 | H06093 | 0.09 | 0.32 | 3.55 | 1.03 | 11.31 | 1.26 | 0.61 | 1 |
| 78 | 284701 | N64840 | 20.71 | 73.02 | 3.53 | 7.91 | 1.97 | 1.81 | 2.41 | 1 |
| 79 | 23790 | T77410 | 0.87 | 3.01 | 3.48 | 1.73 | 1.14 | 2.20 | 8.86 | 1 |
| 80 | 324313 | W47487 | 0.06 | 0.21 | 3.44 | 0.40 | 12.18 | 0.70 | 0.48 | 1 |
| 81 | 270343 | N33041 | 0.22 | 0.73 | 3.35 | 10.97 | 0.92 | 0.66 | 0.84 | 1 |
| 82 | 122782 | T99719 | 0.04 | 0.15 | 3.32 | 11.67 | 0.96 | 0.44 | 0.20 | 1 |
| 83 | 788386 | AA456413 | 0.89 | 2.94 | 3.31 | 1.64 | 0.92 | 0.86 | 9.83 | 1 |
| 84 | 325128 | W48762 | 2.61 | 8.46 | 3.24 | 11.16 | 0.73 | 0.34 | 0.73 | 1 |
| 85 | 192401 | H38425 | 0.27 | 0.85 | 3.18 | 0.87 | 2.00 | 0.96 | 8.89 | 1 |
| 86 | 43207 | H12981 | 0.48 | 1.53 | 3.16 | 0.83 | 9.88 | 0.78 | 1.16 | 1 |
| 87 | 491001 | AA136808 | 16.11 | 48.46 | 3.01 | 6.88 | 1.70 | 2.68 | 0.79 | 1 |
| 88 | 1493160 | AA878880 | 2.32 | 6.88 | 2.97 | 8.81 | 1.47 | 1.13 | 0.45 | 1 |
| 89 | 307174 | N91797 | 4.48 | 13.27 | 2.96 | 7.61 | 1.30 | 1.64 | 1.30 | 1 |
| 90 | 277423 | N47738 | 0.81 | 2.40 | 2.96 | 8.34 | 1.20 | 1.08 | 1.20 | 1 |
| 91 | 50182 | H17882 | 0.33 | 0.93 | 2.84 | 7.27 | 1.81 | 1.00 | 1.27 | 1 |
| 92 | 176904 | H44861 | 1.08 | 2.99 | 2.81 | 2.20 | 0.57 | 7.98 | 0.51 | 1 |
| 93 | 306540 | W31255 | 1.11 | 3.09 | 2.77 | 0.92 | 0.90 | 2.38 | 6.89 | 1 |
| 94 | 123331 | R00480 | 1.76 | 4.88 | 2.77 | 0.56 | 7.08 | 1.72 | 1.71 | 1 |
| 95 | 592707 | AA160606 | 0.03 | 0.09 | 2.76 | 6.45 | 2.28 | 0.77 | 1.55 | 1 |
| 96 | 512116 | AA133721 | 2.28 | 6.25 | 2.75 | 4.47 | 2.89 | 2.52 | 1.10 | 1 |
| 97 | 842848 | AA486281 | 12.01 | 32.44 | 2.70 | 5.87 | 2.16 | 0.62 | 2.15 | 1 |
| 98 | 897901 | AA598659 | 0.91 | 2.47 | 2.70 | 4.79 | 2.01 | 1.70 | 2.30 | 1 |
| 99 | 365227 | AA024898 | 1.07 | 2.89 | 2.70 | 5.78 | 1.41 | 1.42 | 2.18 | 1 |
| 100 | 435567 | AA701931 | 0.88 | 2.38 | 2.69 | 1.67 | 1.47 | 0.98 | 6.67 | 1 |
| 101 | 1481684 | AA885311 | 0.82 | 2.19 | 2.88 | 7.75 | 1.13 | 1.12 | 0.71 | 1 |
| 102 | 49830 | H29258 | 0.94 | 2.51 | 2.67 | 6.78 | 0.87 | 1.48 | 1.74 | 1 |
| 103 | 283208 | N51387 | 1.41 | 3.75 | 2.66 | 1.92 | 1.14 | 0.98 | 6.57 | 1 |
| 104 | 289599 | N79738 | 8.12 | 21.09 | 2.60 | 4.83 | 1.28 | 2.34 | 1.96 | 1 |
| 105 | 230560 | H75860 | 0.28 | 0.73 | 2.60 | 6.99 | 1.63 | 0.92 | 0.94 | 1 |
| 106 | 431296 | AA682631 | 1.88 | 4.25 | 2.56 | 4.00 | 1.76 | 2.75 | 1.75 | 1 |
| 107 | 32381 | R17982 | 3.00 | 7.65 | 2.55 | 5.46 | 2.25 | 1.08 | 1.41 | 1 |
| 108 | 345670 | W78803 | 1.23 | 3.12 | 2.54 | 4.88 | 2.48 | 1.45 | 1.54 | 1 |
| 109 | 196257 | R92601 | 2.99 | 7.59 | 2.54 | 1.46 | 1.12 | 4.84 | 2.93 | 1 |
| 110 | 361363 | | 5.71 | 14.37 | 2.52 | 5.73 | 0.77 | 1.58 | 2.01 | 1 |
| 111 | 359781 | AA010932 | 5.37 | 13.42 | 2.50 | 6.60 | 1.48 | 0.77 | 1.15 | 1 |
| 112 | 882511 | AA676470 | 0.03 | 0.07 | 2.50 | 3.52 | 2.39 | 1.72 | 2.36 | 1 |
| 113 | 811766 | AA4430041 | 3.72 | 34.17 | 2.49 | 5.66 | 1.81 | 0.69 | 1.80 | 1 |
| 114 | 53162 | R15409 | 0.63 | 1.57 | 2.48 | 0.78 | 0.93 | 1.41 | 6.80 | 1 |
| 115 | 120162 | T95274 | 1.05 | 2.60 | 2.47 | 1.13 | 2.51 | 2.86 | 3.38 | 1 |
| 116 | 1470048 | AA865464 | 2.41 | 5.92 | 2.45 | 1.38 | 3.23 | 2.51 | 2.70 | 1 |
| 117 | 1056203 | AA621031 | 9.53 | 23.38 | 2.45 | 8.20 | 0.88 | 0.50 | 0.26 | 1 |
| 118 | 884514 | AA829991 | 2.65 | 6.48 | 2.45 | 6.74 | 1.47 | 1.03 | 0.55 | 1 |
| 119 | 837908 | AA434362 | 3.13 | 7.65 | 2.44 | 1.20 | 1.90 | 4.85 | 1.81 | 1 |
| 120 | 453218 | AA704844 | 0.05 | 0.13 | 2.44 | 5.69 | 1.28 | 1.45 | 1.33 | 1 |
| 121 | 703930 | AA278408 | 1.08 | 2.62 | 2.43 | 2.68 | 2.05 | 3.61 | 1.39 | 1 |
| 122 | 379920 | AA778098 | 1.63 | 3.96 | 2.43 | 2.28 | 3.76 | 2.19 | 1.49 | 1 |
| 123 | 739237 | AA421389 | 2.58 | 6.17 | 2.41 | 3.35 | 1.88 | 2.30 | 2.10 | 1 |
| 124 | 1636495 | AA999953 | 0.69 | 1.65 | 2.39 | 0.80 | 6.02 | 2.03 | 0.72 | 1 |
| 125 | 265102 | N27992 | 1.91 | 4.56 | 2.39 | 6.32 | 1.32 | 1.21 | 0.69 | 1 |
| 126 | 299412 | N76101 | 1.96 | 4.66 | 2.37 | 3.80 | 2.52 | 1.94 | 1.24 | 1 |
| 127 | 1055737 | AA628146 | 1.02 | 2.42 | 2.37 | 0.78 | 1.00 | 0.72 | 6.99 | 1 |
| 128 | 25590 | R12183 | 0.16 | 0.38 | 2.36 | 5.15 | 1.11 | 1.60 | 1.59 | 1 |
| 129 | 396111 | AA757732 | 0.74 | 1.74 | 2.35 | 0.61 | 1.15 | 0.98 | 6.65 | 1 |
| 130 | 206838 | R98064 | 12.93 | 30.32 | 2.34 | 4.58 | 0.52 | 1.88 | 2.40 | 1 |
| 131 | 130046 | R11587 | 11.15 | 26.12 | 2.34 | 1.88 | 1.29 | 4.92 | 1.29 | 1 |
| 132 | 1048794 | AA621324 | 0.23 | 0.54 | 2.34 | 1.96 | 4.69 | 1.83 | 0.89 | 1 |
| 133 | 292071 | W02342 | 0.06 | 0.13 | 2.33 | 6.89 | 0.65 | 0.53 | 1.26 | 1 |
| 134 | 269411 | N26183 | 2.88 | 6.68 | 2.32 | 6.08 | 1.41 | 1.25 | 0.54 | 1 |
| 135 | 392405 | AA708201 | 4.29 | 9.92 | 2.31 | 4.59 | 0.87 | 2.50 | 1.28 | 1 |
| 136 | 627688 | AA197344 | 10.20 | 23.49 | 2.30 | 3.93 | 2.66 | 1.79 | 0.83 | 1 |
| 137 | 1325751 | AA873089 | 0.76 | 1.74 | 2.30 | 5.17 | 1.74 | 1.52 | 0.78 | 1 |
| 138 | 345055 | W76331 | 3.90 | 8.94 | 2.29 | 1.59 | 3.23 | 2.27 | 2.08 | 1 |
| 139 | 757135 | AA444111 | 0.06 | 0.13 | 2.29 | 5.80 | 1.62 | 1.21 | 0.52 | 1 |
| 140 | 455179 | AA676899 | 1.82 | 4.17 | 2.29 | 2.02 | 1.23 | 5.15 | 0.76 | 1 |
| 141 | 429678 | AA011593 | 8.32 | 19.01 | 2.29 | 4.39 | 2.77 | 1.55 | 0.43 | 1 |
| 142 | 49687 | H15250 | 0.58 | 1.33 | 2.28 | 2.29 | 1.86 | 3.12 | 1.85 | 1 |
| 143 | 33096 | R44776 | 0.33 | 0.75 | 2.28 | 6.15 | 0.75 | 1.45 | 0.76 | 1 |
| 144 | 399577 | AA733090 | 1.92 | 4.37 | 2.28 | 1.56 | 3.29 | 2.79 | 1.46 | 1 |
| 145 | 260187 | N45398 | 1.86 | 4.23 | 2.27 | 4.74 | 1.80 | 1.20 | 1.35 | 1 |
| 146 | 743026 | AA406059 | 0.48 | 1.08 | 2.27 | 1.21 | 2.82 | 1.63 | 3.42 | 1 |
| 147 | 309929 | N95435 | 0.43 | 0.98 | 2.26 | 3.67 | 1.36 | 2.50 | 1.52 | 1 |
| 148 | 162077 | H25689 | 0.68 | 1.52 | 2.26 | 6.43 | 1.13 | 0.95 | 0.51 | 1 |
| 149 | 245936 | N55430 | 0.42 | 0.94 | 2.24 | 0.88 | 6.45 | 0.84 | 0.80 | 1 |
| 150 | 897901 | AA598659 | 0.95 | 2.12 | 2.24 | 3.81 | 1.57 | 1.71 | 1.86 | 1 |
| 151 | 454798 | AA677295 | 1.41 | 3.14 | 2.23 | 2.99 | 1.13 | 3.14 | 1.66 | 1 |
| 152 | 1055217 | AA621457 | 0.34 | 0.76 | 2.23 | 6.07 | 1.02 | 1.03 | 0.81 | 1 |
| 153 | 376839 | AA047618 | 0.02 | 0.05 | 2.21 | 4.18 | 1.67 | 1.44 | 1.55 | 1 |
| 154 | 1584505 | AA971641 | 4.96 | 10.94 | 2.21 | 4.93 | 1.92 | 1.44 | 0.53 | 1 |
| 155 | 186623 | R83896 | 1.53 | 3.38 | 2.20 | 0.68 | 2.59 | 2.50 | 3.05 | 1 |
| 156 | 704227 | AA279409 | 0.65 | 1.43 | 2.20 | 1.28 | 0.86 | 1.15 | 5.49 | 1 |
| 157 | 504420 | AA142913 | 3.50 | 7.66 | 2.19 | 5.06 | 0.67 | 1.33 | 1.70 | 1 |
| 158 | 364173 | AA021434 | 0.02 | 0.05 | 2.18 | 0.80 | 1.13 | 1.04 | 5.74 | 1 |
| 159 | 428928 | AA004973 | 3.66 | 7.96 | 2.17 | 4.62 | 1.55 | 1.34 | 1.18 | 1 |
| 160 | 297682 | N69876 | 0.83 | 1.80 | 2.17 | 3.71 | 2.34 | 1.52 | 1.09 | 1 |
| 161 | 610374 | AA171899 | 13.37 | 28.92 | 2.16 | 3.29 | 2.19 | 2.08 | 1.09 | 1 |
| 162 | 502436 | AA134862 | 2.43 | 5.24 | 2.16 | 2.04 | 1.13 | 1.37 | 4.10 | 1 |
| 163 | 795322 | AA454165 | 3.87 | 8.31 | 2.15 | 3.19 | 2.24 | 0.92 | 2.24 | 1 |
| 164 | 359661 | AA011095 | 28.28 | 60.65 | 2.14 | 5.41 | 1.13 | 0.92 | 1.13 | 1 |
| 165 | 746148 | AA419612 | 0.57 | 1.22 | 2.14 | 5.44 | 1.29 | 0.90 | 0.95 | 1 |
| 166 | 202521 | H53141 | 2.66 | 5.69 | 2.14 | 3.03 | 1.52 | 1.84 | 2.18 | 1 |
| 167 | 449061 | AA777417 | 0.27 | 0.58 | 2.14 | 0.67 | 1.29 | 5.68 | 0.92 | 1 |
| 168 | 684562 | AA251386 | 5.24 | 11.18 | 2.13 | 2.51 | 1.19 | 1.66 | 3.17 | 1 |
| 169 | 878836 | AA670429 | 0.54 | 1.14 | 2.13 | 0.82 | 0.97 | 5.76 | 0.97 | 1 |
| 170 | 451898 | AA706955 | 0.37 | 0.78 | 2.12 | 1.07 | 1.33 | 1.09 | 4.99 | 1 |
| 171 | 361668 | W96187 | 42.83 | 90.65 | 2.12 | 1.07 | 0.88 | 1.69 | 4.82 | 1 |
| 172 | 199175 | H83283 | 1.56 | 3.29 | 2.11 | 5.25 | 1.29 | 1.07 | 0.84 | 1 |
| 173 | 745524 | AA626242 | 6.45 | 13.63 | 2.11 | 1.60 | 0.67 | 4.86 | 1.32 | 1 |
| 174 | 430235 | AA010223 | 6.39 | 13.41 | 2.10 | 3.70 | 1.89 | 0.92 | 1.89 | 1 |
| 175 | 826099 | AA521414 | 1.53 | 3.21 | 2.10 | 0.89 | 1.10 | 1.88 | 4.53 | 1 |
| 176 | 280508 | N47308 | 1.12 | 2.34 | 2.09 | 4.19 | 1.04 | 1.47 | 1.65 | 1 |
| 177 | 813488 | AA455565 | 13.37 | 27.88 | 2.08 | 2.34 | 1.65 | 1.24 | 3.11 | 1 |
| 178 | 788574 | AA453040 | 1.28 | 2.66 | 2.06 | 1.31 | 1.18 | 3.06 | 2.79 | 1 |
| 179 | 460189 | AA676948 | 0.38 | 0.79 | 2.07 | 4.30 | 1.36 | 1.24 | 1.39 | 1 |
| 180 | 1493107 | AA876375 | 17.47 | 36.21 | 2.07 | 2.19 | 3.23 | 2.62 | 0.25 | 1 |
| 181 | 290702 | N71769 | 0.33 | 0.69 | 2.07 | 1.94 | 0.88 | 1.57 | 3.92 | 1 |
| 182 | 624379 | AA187789 | 0.93 | 1.92 | 2.07 | 5.14 | 0.73 | 0.96 | 1.44 | 1 |
| 183 | 469989 | AA030046 | 1.69 | 3.48 | 2.06 | 1.18 | 1.79 | 3.50 | 1.78 | 1 |
| 184 | 341336 | W58013 | 1.04 | 2.14 | 2.06 | 5.47 | 1.05 | 1.09 | 0.62 | 1 |
| 185 | 35575 | R45964 | 8.23 | 16.88 | 2.05 | 0.93 | 0.60 | 0.71 | 5.97 | 1 |
| 186 | 470393 | AA031513 | 4.69 | 9.61 | 2.05 | 0.20 | 7.72 | 0.13 | 0.15 | 1 |
| 187 | 359713 | AA011185 | 3.29 | 6.73 | 2.05 | 3.72 | 1.29 | 1.68 | 1.50 | 1 |
| 188 | 781091 | AA430242 | 0.53 | 1.09 | 2.04 | 2.42 | 1.36 | 3.03 | 1.36 | 1 |
| 189 | 46916 | H09996 | 0.13 | 0.27 | 2.04 | 1.18 | 5.44 | 0.70 | 0.84 | 1 |
| 190 | 78946 | T61792 | 1.01 | 2.06 | 2.04 | 3.74 | 1.12 | 2.17 | 1.12 | 1 |
| 191 | 460114 | AA676840 | 1.64 | 3.34 | 2.04 | 3.01 | 1.74 | 1.67 | 1.74 | 1 |
| 192 | 1323328 | AA872602 | 0.37 | 0.75 | 2.03 | 0.84 | 4.37 | 1.57 | 1.34 | 1 |
| 193 | 504927 | AA151002 | 8.88 | 18.02 | 2.03 | 0.16 | 5.34 | 0.12 | 2.51 | 1 |
| 194 | 1574594 | AA968896 | 6.75 | 13.69 | 2.03 | 1.27 | 2.82 | 3.10 | 0.94 | 1 |
| 195 | 264597 | N20328 | 2.35 | 4.76 | 2.03 | 4.18 | 2.51 | 0.99 | 0.44 | 1 |
| 196 | 897822 | AA598572 | 0.38 | 0.77 | 2.03 | 0.98 | 4.23 | 1.23 | 1.67 | 1 |
| 197 | 1031976 | AA609983 | 2.60 | 5.26 | 2.03 | 4.55 | 1.63 | 1.34 | 0.59 | 1 |
| 198 | 595001 | AA164782 | 2.11 | 4.26 | 2.02 | 4.11 | 1.81 | 1.49 | 0.69 | 1 |
| 199 | 491763 | AA150507 | 0.68 | 1.38 | 2.02 | 3.08 | 2.01 | 0.99 | 2.01 | 1 |
| 200 | 324885 | W48700 | 13.11 | 26.52 | 2.02 | 3.94 | 1.97 | 1.52 | 0.65 | 1 |
| 201 | 229573 | H67292 | 4.84 | 9.77 | 2.02 | 2.57 | 3.14 | 1.72 | 0.65 | 1 |
| 202 | 824588 | AA490985 | 3.71 | 7.47 | 2.02 | 3.79 | 2.06 | 1.53 | 0.68 | 1 |
| 203 | 129148 | R10948 | 1.57 | 3.17 | 2.02 | 1.28 | 1.19 | 4.20 | 1.39 | 1 |
| 204 | 46453 | H09749 | 0.12 | 0.24 | 2.01 | 4.88 | 1.22 | 1.31 | 0.62 | 1 |
| 205 | 113284 | T83657 | 2.93 | 5.87 | 2.00 | 3.60 | 1.73 | 1.35 | 1.34 | 1 |
| 206 | 283191 | N51362 | 4.68 | 9.35 | 2.00 | 3.49 | 0.76 | 2.50 | 1.24 | 1 |
| 207 | 134265 | R31114 | 0.45 | 0.91 | 1.99 | 4.80 | 0.96 | 1.20 | 1.02 | 1 |
| 208 | 685127 | AA195601 | 9.63 | 19.17 | 1.99 | 3.49 | 1.88 | 1.69 | 0.90 | 1 |
| 209 | 127063 | R07948 | 1.32 | 2.63 | 1.99 | 1.56 | 4.31 | 1.29 | 0.79 | 1 |
| 210 | 327247 | W02073 | 0.08 | 0.16 | 1.98 | 1.09 | 5.08 | 1.17 | 0.59 | 1 |
| 211 | 268837 | N26008 | 5.41 | 10.70 | 1.98 | 1.71 | 1.38 | 1.20 | 3.62 | 1 |
| 212 | 1505688 | AA879435 | 1.93 | 3.81 | 1.98 | 0.81 | 0.80 | 4.84 | 1.46 | 1 |
| 213 | 855634 | AA664105 | 2.48 | 4.90 | 1.98 | 1.29 | 2.01 | 3.49 | 1.11 | 1 |
| 214 | 322843 | W15319 | 4.16 | 8.22 | 1.98 | 4.19 | 1.69 | 1.22 | 0.80 | 1 |
| 215 | 290346 | N62293 | 1.85 | 3.66 | 1.98 | 2.02 | 1.44 | 1.25 | 3.20 | 1 |
| 216 | 811609 | AA455825 | 0.13 | 0.25 | 1.97 | 3.98 | 1.55 | 1.07 | 1.30 | 1 |
| 217 | 69272 | | 0.04 | 0.07 | 1.97 | 4.53 | 0.76 | 1.82 | 0.75 | 1 |
| 218 | 327495 | W32729 | 0.04 | 0.09 | 1.97 | 5.03 | 0.95 | 1.38 | 0.51 | 1 |
| 219 | 686664 | AA259115 | 1.40 | 2.75 | 1.96 | 3.92 | 1.33 | 1.09 | 1.50 | 1 |
| 220 | 610006 | AA169173 | 0.87 | 1.71 | 1.96 | 3.22 | 1.54 | 1.28 | 1.78 | 1 |
| 221 | 190950 | H38263 | 0.97 | 1.89 | 1.95 | 1.40 | 1.11 | 3.99 | 1.31 | 1 |
| 222 | 729953 | AA399074 | 17.34 | 33.80 | 1.95 | 1.22 | 3.06 | 2.79 | 0.73 | 1 |
| 223 | 726699 | AA399433 | 0.67 | 1.30 | 1.94 | 0.87 | 1.63 | 3.96 | 1.30 | 1 |
| 224 | 770319 | AA434454 | 0.78 | 1.51 | 1.94 | 0.36 | 0.40 | 0.42 | 6.57 | 1 |
| 225 | 43966 | H04828 | 0.01 | 0.02 | 1.93 | 0.00 | 3.64 | 2.03 | 2.06 | 1 |
| 226 | 743828 | AA634379 | 2.44 | 4.71 | 1.93 | 1.86 | 3.99 | 1.05 | 0.82 | 1 |
| 227 | 23219 | T77007 | 0.28 | 0.54 | 1.93 | 4.75 | 1.02 | 0.93 | 1.01 | 1 |
| 228 | 49858 | H29290 | 0.53 | 1.01 | 1.92 | 4.71 | 0.98 | 1.03 | 0.97 | 1 |
| 229 | 24632 | R39364 | 0.21 | 0.41 | 1.92 | 4.62 | 0.94 | 1.19 | 0.93 | 1 |
| 230 | 785816 | AA449048 | 2.16 | 4.14 | 1.92 | 1.20 | 0.82 | 2.59 | 3.07 | 1 |
| 231 | 51420 | H20717 | 0.35 | 0.68 | 1.92 | 4.33 | 1.10 | 1.15 | 1.09 | 1 |
| 232 | 124137 | R01245 | 1.03 | 1.96 | 1.91 | 3.95 | 2.06 | 0.76 | 0.89 | 1 |
| 233 | 789091 | AA453105 | 28.91 | 55.28 | 1.91 | 3.55 | 2.00 | 1.00 | 1.10 | 1 |
| 234 | 753663 | AA478606 | 3.28 | 6.25 | 1.91 | 3.70 | 1.74 | 1.26 | 0.93 | 1 |
| 235 | 151597 | H03146 | 0.95 | 1.81 | 1.90 | 1.10 | 1.80 | 3.06 | 1.65 | 1 |
| 236 | 346966 | W79069 | 0.29 | 0.56 | 1.90 | 1.07 | 1.14 | 4.27 | 1.13 | 1 |
| 237 | 197056 | R92801 | 4.71 | 8.94 | 1.90 | 3.66 | 1.77 | 1.19 | 0.97 | 1 |
| 238 | 132140 | R23609 | 1.73 | 3.29 | 1.90 | 0.75 | 1.18 | 3.27 | 2.39 | 1 |
| 239 | 84078 | T71061 | 2.13 | 4.03 | 1.89 | 0.05 | 6.77 | 0.67 | 0.09 | 1 |
| 240 | 78680 | T61851 | 1.12 | 2.11 | 1.89 | 0.85 | 1.03 | 1.85 | 3.84 | 1 |
| 241 | 788136 | AA453531 | 3.97 | 7.49 | 1.89 | 3.48 | 1.68 | 1.19 | 1.20 | 1 |
| 242 | 462926 | AA682321 | 0.47 | 0.89 | 1.88 | 1.01 | 1.70 | 1.50 | 3.33 | 1 |
| 243 | 784016 | AA443698 | 28.27 | 53.13 | 1.88 | 3.38 | 2.06 | 1.46 | 0.61 | 1 |
| 244 | 813719 | AA453863 | 1.14 | 2.13 | 1.88 | 1.04 | 1.09 | 4.52 | 0.86 | 1 |
| 245 | 201641 | R98344 | 1.40 | 2.63 | 1.88 | 1.20 | 0.56 | 4.05 | 1.70 | 1 |
| 246 | 590253 | AA155787 | 1.88 | 3.49 | 1.85 | 3.06 | 1.96 | 1.38 | 1.01 | 1 |
| 247 | 739578 | AA476961 | 4.47 | 8.26 | 1.85 | 3.36 | 1.38 | 1.44 | 1.21 | 1 |
| 248 | 265853 | N20989 | 0.59 | 1.09 | 1.85 | 1.83 | 0.87 | 3.79 | 0.89 | 1 |
| 249 | 452466 | AA704802 | 0.75 | 1.38 | 1.84 | 1.07 | 1.31 | 1.70 | 3.29 | 1 |
| 250 | 868652 | AA664406 | 22.82 | 41.96 | 1.84 | 3.83 | 1.23 | 1.07 | 1.22 | 1 |
| 251 | 1031203 | AA609982 | 0.29 | 0.53 | 1.83 | 3.78 | 1.06 | 1.44 | 1.05 | 1 |
| 252 | 23000 | R38645 | 2.86 | 5.23 | 1.83 | 0.26 | 4.00 | 0.16 | 2.90 | 1 |
| 253 | 413300 | AA772497 | 1.04 | 1.90 | 1.83 | 0.46 | 0.73 | 0.85 | 5.28 | 1 |
| 254 | 430763 | AA677984 | 0.86 | 1.56 | 1.83 | 0.93 | 1.46 | 1.62 | 3.29 | 1 |
| 255 | 293328 | N91744 | 1.64 | 2.99 | 1.82 | 1.17 | 0.71 | 3.89 | 1.52 | 1 |
| 256 | 713019 | AA282594 | 1.36 | 2.48 | 1.82 | 3.18 | 1.62 | 1.84 | 0.63 | 1 |
| 257 | 770216 | AA434117 | 1.48 | 2.68 | 1.82 | 1.30 | 0.84 | 2.00 | 3.13 | 1 |
| 258 | 454896 | AA677397 | 6.74 | 12.19 | 1.81 | 3.23 | 1.65 | 1.22 | 1.14 | 1 |
| 259 | 417691 | W88623 | 1.55 | 2.78 | 1.79 | 3.84 | 1.46 | 1.11 | 0.96 | 1 |
| 260 | 951022 | AA620421 | 1.22 | 2.18 | 1.79 | 3.35 | 1.44 | 1.72 | 0.66 | 1 |
| 261 | 782797 | AA448194 | 1.29 | 2.31 | 1.79 | 3.26 | 1.22 | 1.19 | 1.47 | 1 |
| 262 | 277112 | N46759 | 1.42 | 2.53 | 1.78 | 1.53 | 1.28 | 0.73 | 3.60 | 1 |
| 263 | 1466485 | AA885052 | 0.01 | 0.03 | 1.78 | 5.61 | 0.58 | 0.82 | 0.11 | 1 |
| 264 | 142944 | R71124 | 1.57 | 2.80 | 1.78 | 4.15 | 1.07 | 1.26 | 0.63 | 1 |
| 265 | 470216 | AA030004 | 4.81 | 8.55 | 1.78 | 3.92 | 0.86 | 1.06 | 1.44 | 1 |
| 266 | 206172 | H61082 | 0.49 | 0.87 | 1.77 | 3.22 | 1.27 | 1.27 | 1.33 | 1 |
| 267 | 1048702 | AA620611 | 2.31 | 4.09 | 1.77 | 3.27 | 1.93 | 1.17 | 0.71 | 1 |
| 268 | 40031 | R53342 | 4.46 | 7.89 | 1.77 | 0.80 | 1.22 | 3.76 | 1.30 | 1 |
| 269 | 1505294 | AA905838 | 1.49 | 2.64 | 1.77 | 1.19 | 1.27 | 3.39 | 1.21 | 1 |
| 270 | 1422794 | AA827405 | 2.92 | 5.15 | 1.77 | 0.55 | 0.81 | 0.90 | 4.80 | 1 |
| 271 | 502593 | AA126682 | 0.14 | 0.26 | 1.76 | 1.28 | 1.38 | 3.02 | 1.39 | 1 |
| 272 | 141815 | R70684 | 3.99 | 7.01 | 1.76 | 3.41 | 0.92 | 1.79 | 0.92 | 1 |
| 273 | 1493079 | AA876354 | 1.59 | 2.78 | 1.75 | 1.40 | 0.92 | 1.44 | 3.23 | 1 |
| 274 | 298648 | W05033 | 6.25 | 10.90 | 1.74 | 1.02 | 1.26 | 1.09 | 3.61 | 1 |
| 275 | 731168 | AA417344 | 1.30 | 2.27 | 1.74 | 3.73 | 1.24 | 1.11 | 0.89 | 1 |
| 276 | 25061 | T81289 | 3.04 | 5.31 | 1.74 | 3.43 | 1.36 | 0.81 | 1.36 | 1 |
| 277 | 838668 | AA457235 | 2.60 | 4.53 | 1.74 | 3.48 | 1.28 | 0.93 | 1.28 | 1 |
| 278 | 488913 | AA057032 | 1.74 | 3.02 | 1.74 | 3.11 | 2.26 | 1.10 | 0.48 | 1 |
| 279 | 127610 | R091791 | 16.74 | 29.04 | 1.74 | 3.75 | 0.59 | 1.53 | 1.07 | 1 |
| 280 | 812053 | AA455980 | 1.23 | 2.13 | 1.73 | 0.81 | 1.60 | 1.38 | 3.13 | 1 |
| 281 | 1056172 | AA620995 | 32.95 | 57.00 | 1.73 | 1.05 | 3.25 | 1.81 | 0.81 | 1 |
| 282 | 24958 | R38943 | 3.00 | 5.19 | 1.73 | 3.45 | 2.31 | 0.87 | 0.28 | 1 |
| 283 | 345701 | W71990 | 0.80 | 1.38 | 1.72 | 1.16 | 1.20 | 1.21 | 3.32 | 1 |
| 284 | 269172 | N26608 | 0.34 | 0.58 | 1.72 | 1.08 | 1.22 | 3.37 | 1.21 | 1 |
| 285 | 798366 | AA459761 | 20.52 | 35.33 | 1.72 | 3.62 | 1.45 | 1.42 | 0.40 | 1 |
| 286 | 682477 | AA255551 | 2.94 | 5.05 | 1.72 | 1.41 | 3.33 | 1.42 | 0.71 | 1 |
| 287 | 249070 | H80063 | 0.82 | 1.42 | 1.72 | 0.91 | 0.78 | 1.47 | 3.71 | 1 |
| 288 | 344108 | W73724 | 4.15 | 7.12 | 1.72 | 3.01 | 1.43 | 1.70 | 0.73 | 1 |
| 289 | 147834 | R81831 | 3.35 | 5.74 | 1.71 | 0.53 | 0.78 | 1.21 | 4.33 | 1 |
| 290 | 436097 | AA700843 | 0.40 | 0.69 | 1.71 | 0.83 | 1.18 | 1.07 | 3.77 | 1 |
| 291 | 435919 | AA701948 | 1.52 | 2.60 | 1.71 | 1.50 | 0.65 | 1.41 | 3.29 | 1 |
| 292 | 25122 | | 0.48 | 0.82 | 1.71 | 0.91 | 0.51 | 4.89 | 0.53 | 1 |
| 293 | 812961 | AA464598 | 1.01 | 1.74 | 1.71 | 0.94 | 0.95 | 1.33 | 3.62 | 1 |
| 294 | 488964 | AA047260 | 36.82 | 62.95 | 1.71 | 3.27 | 1.63 | 1.09 | 0.84 | 1 |
| 295 | 416227 | W86124 | 1.23 | 2.09 | 1.71 | 0.70 | 1.03 | 1.42 | 3.67 | 1 |
| 296 | 41608 | R54179 | 0.19 | 0.33 | 1.70 | 4.47 | 0.57 | 0.95 | 0.83 | 1 |
| 297 | 813149 | AA456695 | 12.62 | 21.50 | 1.70 | 3.29 | 1.50 | 0.99 | 1.05 | 1 |
| 298 | 1684274 | A1003028 | 3.51 | 5.98 | 1.70 | 0.95 | 4.85 | 0.76 | 0.25 | 1 |
| 299 | 256680 | H96392 | 1.91 | 3.24 | 1.70 | 1.42 | 3.15 | 1.78 | 0.45 | 1 |
| 300 | 243159 | H94563 | 1.05 | 1.78 | 1.70 | 3.17 | 1.26 | 1.12 | 1.25 | 1 |
| 301 | 149058 | R82317 | 1.86 | 3.16 | 1.70 | 1.22 | 1.11 | 1.46 | 3.00 | 1 |
| 302 | 220135 | H84734 | 0.03 | 0.06 | 1.69 | 3.52 | 0.87 | 0.62 | 1.76 | 1 |
| 303 | 214713 | H73806 | 0.04 | 0.07 | 1.68 | 3.15 | 2.51 | 0.91 | 0.16 | 1 |
| 304 | 757325 | AA437093 | 0.54 | 0.90 | 1.68 | 0.95 | 1.01 | 1.33 | 3.43 | 1 |
| 305 | 810284 | AA464068 | 6.53 | 10.94 | 1.68 | 3.23 | 0.81 | 1.31 | 1.35 | 1 |
| 306 | 888630 | AA664389 | 28.46 | 47.59 | 1.67 | 3.18 | 1.18 | 1.15 | 1.17 | 1 |
| 307 | 399562 | AA733061 | 3.54 | 5.91 | 1.67 | 3.11 | 1.21 | 1.65 | 0.72 | 1 |
| 308 | 731255 | AA420992 | 5.28 | 8.82 | 1.67 | 3.66 | 0.80 | 1.23 | 0.99 | 1 |
| 309 | 1466942 | AA883114 | 0.51 | 0.85 | 1.67 | 3.31 | 1.11 | 1.01 | 1.24 | 1 |
| 310 | 1461725 | AA884397 | 0.14 | 0.23 | 1.67 | 1.20 | 0.92 | 1.25 | 3.30 | 1 |
| 311 | 1240347 | AA788738 | 0.34 | 0.57 | 1.67 | 3.16 | 1.21 | 1.08 | 1.22 | 1 |
| 312 | 1592715 | AA983362 | 0.09 | 0.15 | 1.66 | 3.86 | 1.00 | 1.09 | 0.71 | 1 |
| 313 | 395417 | AA757417 | 25.66 | 42.64 | 1.66 | 0.80 | 0.68 | 1.29 | 3.88 | 1 |
| 314 | 21634 | T65398 | 1.48 | 2.46 | 1.66 | 3.35 | 0.61 | 1.06 | 1.61 | 1 |
| 315 | 196038 | R89358 | 1.70 | 2.82 | 1.65 | 3.30 | 0.86 | 1.08 | 1.38 | 1 |
| 316 | 193780 | H48138 | 0.35 | 0.59 | 1.65 | 1.04 | 1.07 | 3.11 | 1.38 | 1 |
| 317 | 47527 | H16439 | 2.44 | 4.02 | 1.64 | 1.27 | 3.00 | 1.45 | 0.87 | 1 |
| 318 | 1292593 | AA719257 | 0.49 | 0.80 | 1.64 | 3.21 | 1.20 | 1.06 | 1.11 | 1 |
| 319 | 754313 | AA479512 | 4.83 | 7.92 | 1.64 | 3.23 | 1.40 | 1.32 | 0.62 | 1 |
| 320 | 705064 | AA279990 | 0.43 | 0.71 | 1.64 | 0.78 | 1.57 | 1.14 | 3.05 | 1 |
| 321 | 48454 | H15040 | 0.17 | 0.28 | 1.63 | 3.06 | 0.58 | 1.21 | 1.68 | 1 |
| 322 | 825031 | AA504239 | 7.43 | 12.12 | 1.63 | 3.22 | 2.19 | 0.77 | 0.34 | 1 |
| 323 | 212325 | H68655 | 0.46 | 0.76 | 1.63 | 3.72 | 1.37 | 0.75 | 0.68 | 1 |
| 324 | 32794 | R43553 | 5.83 | 9.47 | 1.62 | 3.08 | 0.59 | 1.24 | 1.58 | 1 |
| 325 | 491415 | AA150422 | 3.54 | 5.76 | 1.62 | 3.80 | 0.84 | 1.03 | 0.84 | 1 |
| 326 | 32509 | R43053 | 7.64 | 12.39 | 1.62 | 0.09 | 0.57 | 0.28 | 5.54 | 1 |
| 327 | 360885 | AA012867 | 11.02 | 17.82 | 1.62 | 3.52 | 1.20 | 0.82 | 0.94 | 1 |
| 328 | 814460 | AA459472 | 0.55 | 0.89 | 1.61 | 0.80 | 3.28 | 1.19 | 1.18 | 1 |
| 329 | 856289 | AA774685 | 2.45 | 3.93 | 1.61 | 3.88 | 1.15 | 0.58 | 0.83 | 1 |
| 330 | 344272 | W73748 | 2.02 | 3.25 | 1.61 | 3.11 | 0.96 | 1.25 | 1.11 | 1 |
| 331 | 841624 | AA487676 | 1.31 | 2.10 | 1.61 | 0.71 | 0.77 | 4.10 | 0.85 | 1 |
| 332 | 530954 | AA070435 | 3.00 | 4.80 | 1.60 | 3.08 | 1.45 | 1.23 | 0.65 | 1 |
| 333 | 279613 | N48913 | 0.21 | 0.33 | 1.60 | 3.61 | 1.04 | 0.94 | 0.80 | 1 |
| 334 | 502818 | AA156926 | 3.72 | 5.92 | 1.59 | 0.73 | 0.86 | 0.91 | 3.88 | 1 |
| 335 | 742132 | AA406019 | 2.03 | 3.22 | 1.59 | 1.04 | 0.84 | 3.56 | 0.91 | 1 |
| 336 | 813755 | AA453900 | 1.86 | 2.95 | 1.59 | 3.39 | 1.61 | 0.88 | 0.48 | 1 |
| 337 | 531862 | AA116061 | 16.93 | 26.88 | 1.59 | 3.03 | 1.25 | 1.23 | 0.84 | 1 |
| 338 | 28270 | R37357 | 1.43 | 2.27 | 1.58 | 0.99 | 3.05 | 1.60 | 0.70 | 1 |
| 339 | 594266 | AA169606 | 4.86 | 7.70 | 1.56 | 3.12 | 1.38 | 1.04 | 0.79 | 1 |
| 340 | 47378 | H11036 | 1.54 | 2.42 | 1.58 | 3.26 | 1.24 | 0.57 | 1.24 | 1 |
| 341 | 703943 | AA279145 | 7.55 | 11.90 | 1.58 | 3.83 | 0.81 | 0.88 | 0.79 | 1 |
| 342 | 813256 | AA456377 | 0.86 | 1.36 | 1.57 | 0.74 | 4.59 | 0.41 | 0.55 | 1 |
| 343 | 160730 | H24580 | 1.12 | 1.75 | 1.56 | 3.04 | 0.58 | 1.31 | 1.32 | 1 |
| 344 | 52002 | H23548 | 1.14 | 1.76 | 1.55 | 3.24 | 0.86 | 0.91 | 1.19 | 1 |
| 345 | 204251 | H59188 | 1.38 | 2.13 | 1.55 | 3.28 | 1.16 | 0.93 | 0.81 | 1 |
| 346 | 308223 | W24646 | 0.79 | 1.22 | 1.55 | 0.78 | 0.90 | 1.06 | 3.45 | 1 |
| 347 | 712888 | AA282159 | 6.43 | 9.93 | 1.54 | 3.59 | 1.45 | 0.66 | 0.47 | 1 |
| 348 | 951108 | AA620466 | 2.04 | 3.15 | 1.54 | 0.58 | 0.79 | 0.86 | 3.94 | 1 |
| 349 | 743187 | AA400002 | 1.45 | 2.24 | 1.54 | 1.00 | 0.70 | 0.68 | 3.79 | 1 |
| 350 | 66952 | T69522 | 0.11 | 0.16 | 1.54 | 0.99 | 0.71 | 3.21 | 1.26 | 1 |
| 351 | 129589 | R16541 | 0.92 | 1.41 | 1.54 | 0.59 | 3.16 | 1.98 | 0.42 | 1 |
| 352 | 461699 | AA682274 | 5.46 | 8.37 | 1.53 | 3.63 | 1.03 | 0.75 | 0.72 | 1 |
| 353 | 452676 | AA779225 | 0.40 | 0.61 | 1.52 | 0.71 | 1.06 | 3.06 | 1.26 | 1 |
| 354 | 196992 | R93124 | 1.44 | 2.19 | 1.52 | 0.64 | 3.67 | 0.94 | 0.83 | 1 |
| 355 | 1501546 | AA886792 | 0.94 | 1.42 | 1.52 | 0.86 | 3.03 | 1.48 | 0.70 | 1 |
| 356 | 754591 | AA406320 | 3.70 | 5.60 | 1.51 | 0.83 | 0.54 | 0.56 | 4.14 | 1 |
| 357 | 70327 | AA278842 | 1.39 | 2.09 | 1.51 | 0.78 | 1.00 | 1.13 | 3.11 | 1 |
| 358 | 1486752 | AA912071 | 1.05 | 1.58 | 1.50 | 0.69 | 0.83 | 0.71 | 3.79 | 1 |
| 359 | 281045 | N50904 | 0.98 | 1.47 | 1.50 | 0.93 | 0.85 | 3.38 | 0.85 | 1 |
| 360 | 505414 | AA161248 | 0.06 | 0.09 | 1.47 | 0.78 | 3.03 | 1.63 | 0.43 | 1 |
| 361 | 179631 | H51122 | 0.50 | 0.73 | 1.47 | 0.68 | 1.09 | 0.74 | 3.36 | 1 |
| 362 | 201125 | R98472 | 9.68 | 14.17 | 1.46 | 3.64 | 1.24 | 0.76 | 0.22 | 1 |
| 363 | 753271 | AA411668 | 1.04 | 1.51 | 1.46 | 0.94 | 0.87 | 0.95 | 3.08 | 1 |
| 364 | 46844 | H10073 | 0.11 | 0.16 | 1.45 | 3.45 | 0.79 | 0.80 | 0.78 | 1 |
| 365 | 453988 | AA779212 | 0.48 | 0.70 | 1.45 | 3.31 | 1.03 | 0.67 | 0.80 | 1 |
| 366 | 141548 | R73088 | 1.37 | 1.98 | 1.44 | 0.86 | 0.70 | 0.51 | 3.69 | 1 |
| 367 | 151184 | H02294 | 2.40 | 3.43 | 1.43 | 0.35 | 0.85 | 0.77 | 3.76 | 1 |
| 368 | 283697 | H99672 | 8.21 | 11.76 | 1.43 | 3.35 | 1.55 | 0.52 | 0.31 | 1 |
| 369 | 845521 | AA644563 | 1.84 | 2.63 | 1.43 | 3.26 | 0.74 | 1.12 | 0.61 | 1 |
| 370 | 428796 | AA004671 | 0.06 | 0.09 | 1.43 | 0.79 | 3.16 | 0.91 | 0.66 | 1 |
| 371 | 124567 | R01937 | 0.08 | 0.11 | 1.43 | 3.11 | 0.76 | 1.14 | 0.69 | 1 |
| 372 | 44300 | H06377 | 2.96 | 4.22 | 1.42 | 1.02 | 0.50 | 0.96 | 3.22 | 1 |
| 373 | 454564 | AA677025 | 0.87 | 1.23 | 1.41 | 3.29 | 0.79 | 0.81 | 0.76 | 1 |
| 374 | 196612 | R92994 | 0.45 | 0.63 | 1.40 | 3.60 | 0.87 | 0.59 | 0.56 | 1 |
| 375 | 665774 | AA193254 | 11.47 | 16.09 | 1.40 | 3.04 | 1.19 | 0.60 | 0.78 | 1 |
| 376 | 416305 | W86183 | 0.80 | 1.11 | 1.40 | 0.88 | 3.65 | 0.57 | 0.50 | 1 |
| 377 | 773483 | AA428014 | 9.13 | 12.76 | 1.40 | 3.08 | 1.03 | 0.71 | 0.77 | 1 |
| 378 | 244310 | N54788 | 0.57 | 0.80 | 1.40 | 3.47 | 1.05 | 0.48 | 0.58 | 1 |
| 379 | 195751 | R89082 | 1.31 | 1.83 | 1.39 | 3.29 | 1.17 | 0.74 | 0.37 | 1 |
| 380 | 1474284 | AA922309 | 2.61 | 3.59 | 1.38 | 0.53 | 3.15 | 0.82 | 0.99 | 1 |
| 381 | 451911 | AA706969 | 1.35 | 1.84 | 1.36 | 0.74 | 0.80 | 0.69 | 3.23 | 1 |
| 382 | 357264 | W93483 | 1.72 | 2.34 | 1.36 | 0.78 | 0.37 | 0.52 | 3.78 | 1 |
| 383 | 897952 | AA598815 | 11.02 | 14.94 | 1.36 | 3.35 | 1.04 | 0.73 | 0.31 | 1 |
| 384 | 84464 | T73883 | 3.20 | 4.30 | 1.35 | 0.53 | 0.78 | 3.28 | 0.78 | 1 |
| 385 | 277708 | N49574 | 1.13 | 1.52 | 1.34 | 0.34 | 3.48 | 0.80 | 0.75 | 1 |
| 386 | 243741 | N49629 | 2.79 | 3.71 | 1.33 | 0.46 | 3.86 | 0.49 | 0.51 | 1 |
| 387 | 563598 | AA102670 | 1.07 | 1.41 | 1.32 | 0.66 | 3.67 | 0.54 | 0.39 | 1 |
| 388 | 376043 | AA040265 | 0.43 | 0.57 | 1.32 | 3.50 | 0.70 | 0.36 | 0.70 | 1 |
| 389 | 784772 | AA478542 | 1.01 | 1.30 | 1.30 | 0.59 | 3.06 | 0.70 | 0.84 | 1 |
| 390 | 544639 | AA074877 | 4.08 | 5.27 | 1.29 | 3.08 | 0.88 | 0.73 | 0.49 | 1 |
| 391 | 222157 | H85806 | 7.18 | 9.28 | 1.29 | 3.11 | 1.05 | 0.59 | 0.42 | 1 |
| 392 | 284100 | N53421 | 2.03 | 2.54 | 1.25 | 0.85 | 0.53 | 3.10 | 0.52 | 1 |
| 393 | 42373 | R67147 | 5.89 | 7.36 | 1.25 | 3.12 | 0.58 | 0.64 | 0.67 | 1 |
| 394 | 784017 | AA443706 | 0.08 | 0.10 | 1.25 | 0.13 | 0.45 | 4.01 | 0.40 | 1 |
| 395 | 178860 | H49519 | 4.98 | 6.03 | 1.21 | 3.10 | 0.45 | 0.85 | 0.45 | 1 |
| 396 | 741497 | AA401137 | 1.82 | 2.20 | 1.21 | 0.47 | 3.65 | 0.36 | 0.37 | 1 |
| 397 | 380890 | AA058597 | 3.53 | 4.23 | 1.20 | 3.25 | 0.72 | 0.56 | 0.26 | 1 |
| 398 | 1390584 | AA843592 | 23.01 | 25.11 | 1.09 | 3.29 | 0.25 | 0.22 | 0.60 | 1 |
| 399 | 462325 | AA705516 | 13.33 | 14.06 | 1.05 | 0.12 | 3.40 | 0.30 | 0.40 | 1 |
| 400 | 814053 | AA465495 | 17.56 | 15.23 | 0.87 | 0.10 | 3.03 | 0.18 | 0.16 | 1 |

TABLE 2-5
Poor vs Good Clinical Outcome:
Genes upregulated 3 fold or more In at least 2 tumor samples.

| | | | | | | | | Fold | Fold- | |
|---|---|---|---|---|---|---|---|---|---|---|
| | IMAGE | Accession | | | Fold(AvePoor/ | Fold(mpm31/ | Fold(mpm32/ | (mpm33/ | (mpm34/ | # of Tumor |
| Order | Clone ID | No. | AveGood | AvePoor | AveGood) | AveGood) | AveGood) | AveGood) | AveGood) | Samples |
| 1 | 753076 | AA436479 | 1.42 | 42.16 | 29.71 | 2.52 | 57.56 | 34.98 | 23.80 | 3 |
| 2 | 124071 | R02586 | 0.53 | 3.21 | 6.08 | 0.69 | 16.64 | 3.34 | 3.64 | 3 |
| 3 | 346321 | W79647 | 0.64 | 3.29 | 5.17 | 3.69 | 7.75 | 1.51 | 7.73 | 3 |
| 4 | 855624 | AA664101 | 24.18 | 108.55 | 4.49 | 4.12 | 6.82 | 0.22 | 6.80 | 3 |
| 5 | 42325 | R61073 | 13.03 | 52.25 | 4.01 | 4.90 | 5.91 | 4.66 | 0.57 | 3 |
| 6 | 287528 | | 2.13 | 6.32 | 2.97 | 4.00 | 3.29 | 1.30 | 3.29 | 3 |
| 7 | 756595 | AA444051 | 1.46 | 3.89 | 2.67 | 0.45 | 3.61 | 3.01 | 3.60 | 3 |
| 8 | 452374 | AA700876 | 0.64 | 19.98 | 31.26 | 0.24 | 62.30 | 0.37 | 62.14 | 2 |
| 9 | 357105 | W93315 | 0.87 | 8.54 | 9.81 | 0.81 | 25.33 | 12.26 | 0.86 | 2 |
| 10 | 460798 | AA708152 | 1.24 | 6.95 | 5.61 | 0.47 | 17.80 | 3.83 | 0.33 | 2 |
| 11 | 284601 | N76201 | 0.27 | 1.50 | 5.49 | 0.82 | 10.19 | 0.77 | 10.17 | 2 |
| 12 | 460487 | AA677706 | 53.51 | 237.74 | 4.44 | 0.02 | 8.87 | 0.03 | 8.85 | 2 |
| 13 | 248412 | N58558 | 0.86 | 3.63 | 4.25 | 0.95 | 7.66 | 0.75 | 7.64 | 2 |
| 14 | 212188 | H68848 | 0.56 | 2.37 | 4.23 | 0.93 | 6.76 | 2.47 | 6.75 | 2 |
| 15 | 782513 | AA432030 | 4.08 | 16.09 | 3.95 | 1.06 | 1.58 | 6.19 | 6.98 | 2 |
| 16 | 782513 | AA432030 | 2.03 | 7.78 | 3.83 | 1.37 | 1.26 | 6.00 | 6.70 | 2 |
| 17 | 129128 | R10935 | 0.38 | 1.47 | 3.81 | 0.92 | 3.71 | 0.70 | 9.92 | 2 |
| 18 | 854678 | AA630084 | 6.04 | 21.23 | 3.52 | 1.40 | 4.21 | 2.50 | 5.95 | 2 |
| 19 | 362059 | AA001431 | 1.47 | 5.16 | 3.51 | 1.66 | 5.87 | 0.66 | 5.86 | 2 |
| 20 | 454822 | AA677388 | 0.60 | 2.09 | 3.47 | 0.37 | 6.64 | 0.26 | 6.62 | 2 |
| 21 | 308696 | N92842 | 0.05 | 0.16 | 3.28 | 1.59 | 6.55 | 0.00 | 4.97 | 2 |
| 22 | 415766 | W84751 | 1.20 | 3.83 | 3.19 | 0.75 | 5.82 | 0.36 | 5.81 | 2 |
| 23 | 489535 | AA099262 | 3.91 | 12.41 | 3.17 | 5.95 | 0.84 | 1.37 | 4.54 | 2 |
| 24 | 364865 | AA028171 | 0.80 | 2.53 | 3.17 | 3.39 | 2.18 | 4.93 | 2.18 | 2 |
| 25 | 740941 | AA478298 | 53.79 | 169.35 | 3.15 | 1.55 | 5.32 | 0.41 | 5.31 | 2 |
| 26 | 241705 | H91680 | 1.71 | 5.19 | 3.04 | 1.25 | 4.71 | 5.51 | 0.69 | 2 |
| 27 | 754559 | AA406301 | 1.32 | 3.96 | 3.00 | 2.54 | 3.88 | 4.35 | 1.25 | 2 |
| 28 | 487327 | AA045524 | 0.49 | 1.46 | 2.96 | 1.51 | 4.72 | 0.91 | 4.72 | 2 |
| 29 | 843098 | AA488676 | 5.29 | 15.54 | 2.94 | 1.27 | 2.66 | 3.34 | 4.49 | 2 |
| 30 | 298303 | N73958 | 0.22 | 0.65 | 2.94 | 0.99 | 4.79 | 1.18 | 4.79 | 2 |
| 31 | 815542 | AA456886 | 1.41 | 4.08 | 2.90 | 1.32 | 1.80 | 4.07 | 4.42 | 2 |
| 32 | 782537 | AA432049 | 1.48 | 4.26 | 2.87 | 0.83 | 0.86 | 4.85 | 4.95 | 2 |
| 33 | 324437 | W46900 | 1.10 | 3.16 | 2.86 | 0.62 | 5.28 | 0.29 | 5.26 | 2 |
| 34 | 461759 | AA682386 | 0.67 | 1.86 | 2.79 | 1.13 | 4.59 | 0.83 | 4.59 | 2 |
| 35 | 754393 | AA436317 | 0.01 | 0.02 | 2.67 | 5.43 | 3.14 | 2.12 | 0.00 | 2 |
| 36 | 770983 | AA428415 | 16.45 | 43.42 | 2.64 | 1.19 | 3.67 | 2.04 | 3.66 | 2 |
| 37 | 415692 | W84716 | 0.65 | 1.70 | 2.60 | 1.72 | 3.82 | 1.06 | 3.81 | 2 |
| 38 | 435820 | AA701519 | 1.20 | 3.12 | 2.59 | 3.20 | 1.28 | 3.41 | 2.47 | 2 |
| 39 | 823696 | AA489743 | 1.17 | 3.00 | 2.56 | 0.92 | 1.43 | 3.94 | 3.92 | 2 |
| 40 | 811000 | AA485508 | 7.79 | 19.45 | 2.50 | 0.66 | 4.20 | 0.94 | 4.19 | 2 |
| 41 | 754600 | AA411384 | 6.54 | 15.91 | 2.43 | 1.99 | 1.25 | 3.06 | 3.45 | 2 |
| 42 | 41214 | R56898 | 0.08 | 0.19 | 2.43 | 1.43 | 3.22 | 1.85 | 3.21 | 2 |
| 43 | 898096 | AA598795 | 3.43 | 8.05 | 2.35 | 1.11 | 1.22 | 3.21 | 3.86 | 2 |
| 44 | 1473304 | AA916325 | 3.39 | 7.96 | 2.35 | 1.13 | 4.30 | 3.08 | 0.87 | 2 |
| 45 | 359835 | AA011215 | 29.86 | 69.88 | 2.34 | 3.49 | 3.72 | 0.91 | 1.24 | 2 |
| 46 | 430231 | AA010221 | 0.55 | 1.28 | 2.34 | 1.13 | 3.86 | 0.53 | 3.83 | 2 |
| 47 | 376516 | AA041499 | 2.44 | 5.70 | 2.34 | 1.48 | 1.07 | 3.21 | 3.59 | 2 |
| 48 | 323238 | W42812 | 2.09 | 4.88 | 2.34 | 0.57 | 4.18 | 0.41 | 4.17 | 2 |
| 49 | 770880 | AA434502 | 20.98 | 48.48 | 2.31 | 0.42 | 3.59 | 1.65 | 3.58 | 2 |
| 50 | 303099 | W20458 | 1.81 | 4.15 | 2.29 | 1.40 | 3.03 | 1.72 | 3.02 | 2 |
| 51 | 39959 | R53558 | 0.06 | 0.13 | 2.21 | 1.76 | 3.34 | 0.34 | 3.39 | 2 |
| 52 | 138788 | R63646 | 0.49 | 1.05 | 2.16 | 0.94 | 3.49 | 0.72 | 3.49 | 2 |
| 53 | 52071 | H24350 | 0.21 | 0.44 | 2.15 | 1.28 | 3.16 | 0.97 | 3.19 | 2 |
| 54 | 47418 | H11063 | 2.05 | 4.37 | 2.13 | 0.39 | 3.33 | 1.47 | 3.32 | 2 |
| 55 | 787938 | AA452278 | 2.20 | 4.63 | 2.10 | 0.89 | 0.69 | 3.21 | 3.63 | 2 |
| 56 | 796806 | AA461174 | 0.45 | 0.92 | 2.06 | 1.38 | 3.16 | 0.53 | 3.16 | 2 |
| 57 | 223350 | H86554 | 0.70 | 1.35 | 1.93 | 0.44 | 3.45 | 0.36 | 3.44 | 2 |
| 58 | 510136 | AA053684 | 0.05 | 0.09 | 1.88 | 0.16 | 3.22 | 0.95 | 3.20 | 2 |
| 59 | 741977 | AA401441 | 4.15 | 7.71 | 1.86 | 0.53 | 3.20 | 0.51 | 3.19 | 2 |

TABLE 2-6
Poor vs. Good Clinical Outcome: Genes upregulated 5 fold or more in at least 2 tumor samples.

| | | | | | | Fold | Fold | Fold | Fold | Fold | |
|---|---|---|---|---|---|---|---|---|---|---|---|
| | IMAGE | Accession | Tissue | | | (AvePoor/ | (mpm31/ | (mpm32/ | (mpm33/ | (mpm34/ | # of Tumors |
| Order | Clone Id | No. | Distribution | AveGood | AvePoor | AveGood) | AveGood) | AveGood) | AveGood) | AveGood) | Samples |
| 1 | 753076 | AA436479 | thymus | 1.42 | 42.16 | 29.71 | 2.52 | 57.56 | 34.98 | 23.80 | 3 |
| 2 | 452374 | AA700876 | prostate | 0.64 | 19.98 | 31.26 | 0.24 | 62.30 | 0.37 | 62.14 | 2 |
| 3 | 357105 | W93315 | | 0.87 | 8.54 | 9.81 | 0.81 | 25.33 | 12.26 | 0.86 | 2 |
| 4 | 284601 | N76201 | brain | 0.27 | 1.50 | 5.49 | 0.82 | 10.19 | 0.77 | 10.17 | 2 |
| 5 | 346321 | W74079 | spleen | 0.64 | 3.29 | 5.17 | 3.69 | 7.75 | 1.51 | 7.73 | 2 |
| 6 | 855824 | AA664101 | testis | 24.18 | 108.55 | 4.49 | 4.12 | 6.82 | 0.22 | 6.80 | 2 |
| 7 | 460487 | AA677706 | bone | 53.51 | 237.74 | 4.44 | 0.02 | 8.87 | 0.03 | 8.85 | 2 |
| 8 | 248412 | N58558 | liver | 0.86 | 3.63 | 4.25 | 0.95 | 7.66 | 0.75 | 7.64 | 2 |
| 9 | 212188 | H69148 | liver | 0.56 | 2.37 | 4.23 | 0.93 | 6.76 | 2.47 | 6.75 | 2 |
| 10 | 782513 | AA432030 | testis | 4.08 | 16.09 | 3.95 | 1.06 | 1.58 | 6.19 | 6.98 | 2 |
| 11 | 782513 | AA432030 | testis | 2.03 | 7.78 | 3.83 | 1.37 | 1.26 | 6.00 | 6.70 | 2 |
| 12 | 362059 | AA001431 | bone | 1.47 | 5.16 | 3.51 | 1.68 | 5.87 | 0.66 | 5.86 | 2 |
| 13 | 454822 | AA677388 | colon | 0.60 | 2.09 | 3.47 | 0.37 | 6.64 | 0.26 | 6.62 | 2 |
| 14 | 415766 | W84751 | heart | 1.20 | 3.83 | 3.19 | 0.75 | 5.82 | 0.36 | 5.81 | 2 |
| 15 | 740941 | AA478298 | testis | 53.79 | 169.35 | 3.15 | 1.55 | 5.32 | 0.41 | 5.31 | 2 |
| 16 | 324437 | W46900 | bone | 1.10 | 3.16 | 2.86 | 0.62 | 5.28 | 0.29 | 5.26 | 2 |

TABLE 2-7
Good vs. Poor Clinical Outcome: Genes upregulated 3 fold or more in at least 1 tumor sample.

| | | Gen Bank | | | Fold | Fold | Fold | Fold | Fold | Fold | |
|---|---|---|---|---|---|---|---|---|---|---|---|
| | | Accession | | | (AveGood/ | (mpm27/ | (mpm28/ | (mpm29/ | (mpm30/ | (mpm35/ | # of Tumors |
| Order | Clone | Number | AveGood | AvePoor | AvePoor) | AvePoor) | AvePoor) | AvePoor) | AvePoor) | AvePoor) | Samples |
| 1 | 299679 | W05628 | 2.56 | 0.60 | 5.07 | 7.07 | 6.80 | 3.51 | 0.68 | 7.30 | 4 |
| 2 | 811162 | AA488471 | 28.87 | 8.85 | 4.21 | 0.70 | 3.61 | 6.33 | 4.17 | 6.26 | 4 |
| 3 | 471641 | AA034945 | 11.03 | 1.08 | 10.17 | 21.19 | 2.20 | 4.22 | 2.10 | 21.15 | 3 |
| 4 | 281039 | N47717 | 36.87 | 4.42 | 8.29 | 22.87 | 1.40 | 0.27 | 9.85 | 7.08 | 3 |
| 5 | 282089 | N48259 | 27.13 | 3.45 | 7.87 | 22.41 | 0.41 | 0.67 | 7.05 | 8.80 | 3 |
| 6 | 155072 | R71393 | 3.32 | 0.45 | 7.44 | 8.50 | 1.97 | 1.30 | 9.52 | 15.90 | 3 |
| 7 | 208210 | H65288 | 2.70 | 0.42 | 6.48 | 14.00 | 1.98 | 0.98 | 5.30 | 10.17 | 3 |
| 8 | 770068 | AA430545 | 26.72 | 4.62 | 5.78 | 12.05 | 0.87 | 1.90 | 8.30 | 5.96 | 3 |
| 9 | 712950 | AA282273 | 38.22 | 8.94 | 5.50 | 8.84 | 1.04 | 0.35 | 4.37 | 12.92 | 3 |
| 10 | 81331 | T60111 | 4.13 | 0.82 | 5.03 | 11.18 | 1.30 | 0.80 | 8.59 | 3.28 | 3 |
| 11 | 287728 | N59158 | 3.49 | 0.71 | 4.90 | 14.39 | 0.35 | 0.46 | 3.86 | 5.42 | 3 |
| 12 | 41228 | R68953 | 0.05 | 0.01 | 4.38 | 9.07 | 2.53 | 4.48 | 2.14 | 3.71 | 3 |
| 13 | 122345 | T99191 | 0.03 | 0.01 | 4.16 | 12.66 | 4.17 | 0.79 | 0.00 | 3.18 | 3 |
| 14 | 188918 | H43317 | 6.27 | 1.80 | 3.93 | 3.28 | 1.41 | 1.33 | 4.30 | 9.31 | 3 |
| 15 | 1049033 | AA778875 | 2.50 | 0.65 | 3.87 | 4.86 | 2.38 | 4.54 | 2.55 | 5.03 | 3 |
| 16 | 195385 | R88904 | 1.81 | 0.50 | 3.62 | 6.03 | 1.45 | 0.88 | 3.51 | 6.23 | 3 |
| 17 | 50114 | H16743 | 1.48 | 0.43 | 3.43 | 5.36 | 4.18 | 4.79 | 2.28 | 0.54 | 3 |
| 18 | 838811 | AA458975 | 87.41 | 25.99 | 3.38 | 5.71 | 4.22 | 4.22 | 1.32 | 1.35 | 3 |
| 19 | 283198 | N45282 | 5.16 | 1.55 | 3.32 | 7.31 | 0.87 | 1.42 | 3.24 | 3.77 | 3 |
| 20 | 745625 | AA626248 | 4.16 | 1.27 | 3.28 | 5.86 | 1.71 | 0.34 | 4.78 | 3.74 | 3 |
| 21 | 159487 | 815926 | 0.12 | 0.04 | 2.92 | 2.50 | 3.69 | 3.70 | 1.59 | 3.11 | 3 |
| 22 | 1034473 | AA779728 | 76.12 | 27.08 | 2.81 | 2.22 | 3.21 | 3.87 | 0.79 | 3.97 | 3 |
| 23 | 138444 | R88272 | 2.02 | 0.74 | 2.75 | 4.16 | 1.66 | 1.35 | 3.32 | 3.28 | 3 |
| 24 | 378488 | AA777187 | 17.01 | 7.17 | 2.37 | 3.23 | 1.20 | 0.99 | 3.13 | 3.31 | 3 |
| 25 | 787130 | AA424535 | 4.28 | 0.37 | 11.65 | 1.60 | 25.69 | 28.44 | 1.49 | 1.02 | 2 |
| 26 | 307337 | N95228 | 10.81 | 0.96 | 11.29 | 1.65 | 7.47 | 44.45 | 1.65 | 1.22 | 2 |
| 27 | 502625 | AA134578 | 7.27 | 0.90 | 6.08 | 1.44 | 1.15 | 33.58 | 1.04 | 3.17 | 2 |
| 28 | 41889 | R68438 | 1.41 | 0.28 | 5.03 | 1.45 | 5.93 | 15.74 | 1.30 | 0.71 | 2 |
| 29 | 301735 | N90882 | 4.10 | 0.90 | 4.58 | 9.05 | 2.75 | 2.93 | 0.99 | 7.09 | 2 |
| 30 | 138805 | R35051 | 32.22 | 7.39 | 4.38 | 2.55 | 3.53 | 11.13 | 1.94 | 2.65 | 2 |
| 31 | 382278 | AA001219 | 5.53 | 1.41 | 3.91 | 2.64 | 2.34 | 0.78 | 6.56 | 7.23 | 2 |
| 32 | 758708 | AA443903 | 2.58 | 0.67 | 3.84 | 0.50 | 4.59 | 10.09 | 2.99 | 1.01 | 2 |
| 33 | 265028 | N21084 | 1.22 | 0.32 | 3.78 | 1.00 | 4.38 | 11.96 | 0.59 | 0.98 | 2 |
| 34 | 1409509 | AA888929 | 2.84 | 0.75 | 3.78 | 0.36 | 5.86 | 11.75 | 0.59 | 0.31 | 2 |
| 35 | 385707 | AA025434 | 1.94 | 0.53 | 3.68 | 0.58 | 3.38 | 10.11 | 1.36 | 3.00 | 2 |
| 36 | 768953 | AA424920 | 3.43 | 0.94 | 3.86 | 0.96 | 5.41 | 10.03 | 0.93 | 0.97 | 2 |
| 37 | 357828 | W94121 | 1.80 | 0.49 | 3.65 | 5.62 | 0.55 | 0.68 | 2.00 | 9.41 | 2 |
| 38 | 35058 | R45192 | 28.75 | 7.90 | 3.64 | 9.19 | 4.22 | 2.07 | 1.34 | 1.38 | 2 |
| 39 | 145740 | R77948 | 30.23 | 8.55 | 3.53 | 1.47 | 0.52 | 0.73 | 5.51 | 9.44 | 2 |
| 40 | 757200 | AA443987 | 0.08 | 0.02 | 3.51 | 1.63 | 2.44 | 1.32 | 3.97 | 8.17 | 2 |
| 41 | 858575 | AA633658 | 13.79 | 4.11 | 3.36 | 1.01 | 1.91 | 2.38 | 6.90 | 4.57 | 2 |
| 42 | 731469 | AA412417 | 3.83 | 1.14 | 3.35 | 3.72 | 2.54 | 5.00 | 2.74 | 2.75 | 2 |
| 43 | 140197 | R66101 | 2.60 | 0.79 | 3.29 | 0.86 | 3.45 | 10.28 | 0.99 | 0.88 | 2 |
| 44 | 48188 | H09076 | 12.10 | 3.88 | 3.29 | 8.38 | 1.09 | 0.51 | 2.55 | 3.90 | 2 |
| 45 | 123284 | R00276 | 8.05 | 2.46 | 3.28 | 0.59 | 4.91 | 1.61 | 1.60 | 7.68 | 2 |
| 46 | 37980 | R61372 | 0.83 | 0.25 | 3.28 | 8.99 | 3.72 | 2.26 | 0.96 | 0.35 | 2 |
| 47 | 1055707 | AA828113 | 3.79 | 1.18 | 3.23 | 6.07 | 2.96 | 1.26 | 2.50 | 3.34 | 2 |
| 48 | 245277 | N53447 | 7.05 | 2.22 | 3.18 | 8.02 | 1.26 | 0.59 | 2.35 | 3.69 | 2 |
| 49 | 878598 | AA775257 | 5.91 | 1.88 | 3.14 | 7.62 | 1.48 | 0.91 | 2.42 | 3.25 | 2 |
| 50 | 66599 | T67128 | 3.06 | 0.98 | 3.12 | 3.86 | 0.81 | 0.45 | 1.32 | 9.18 | 2 |
| 51 | 782335 | AA432270 | 4.04 | 1.30 | 3.12 | 6.21 | 5.84 | 1.80 | 0.68 | 1.08 | 2 |
| 52 | 193087 | AA47076 | 0.06 | 0.02 | 3.11 | 3.68 | 0.71 | 1.77 | 0.49 | 8.89 | 2 |
| 53 | 151477 | H02837 | 2.32 | 0.75 | 3.09 | 10.04 | 0.89 | 0.57 | 3.20 | 0.75 | 2 |
| 54 | 768020 | AA418748 | 4.17 | 1.36 | 3.06 | 0.74 | 4.10 | 0.74 | 0.60 | 9.12 | 2 |
| 55 | 35329 | R45517 | 1.82 | 0.64 | 2.86 | 3.39 | 2.99 | 4.46 | 1.70 | 1.75 | 2 |
| 56 | 245990 | N55459 | 101.29 | 38.00 | 2.81 | 0.45 | 1.13 | 0.94 | 6.81 | 4.74 | 2 |
| 57 | 288983 | N59816 | 2.12 | 0.75 | 2.81 | 0.82 | 1.41 | 1.52 | 4.53 | 5.77 | 2 |
| 58 | 646037 | AA196979 | 98.80 | 35.15 | 2.81 | 0.92 | 8.21 | 3.16 | 1.58 | 2.19 | 2 |
| 59 | 434952 | AA700680 | 1.23 | 0.45 | 2.77 | 1.83 | 3.75 | 6.22 | 0.82 | 1.24 | 2 |
| 60 | 231574 | H92821 | 2.29 | 0.83 | 2.75 | 6.94 | 3.48 | 1.02 | 0.85 | 1.47 | 2 |
| 61 | 840590 | AA487902 | 48.94 | 17.94 | 2.73 | 4.81 | 1.98 | 0.79 | 1.17 | 4.89 | 2 |
| 62 | 78353 | T58281 | 39.72 | 14.62 | 2.72 | 0.58 | 0.96 | 1.14 | 6.48 | 4.41 | 2 |
| 63 | 1418821 | AA878120 | 3.31 | 1.22 | 2.71 | 4.87 | 1.25 | 1.55 | 2.55 | 3.34 | 2 |
| 64 | 68902 | T69477 | 2.93 | 1.09 | 2.68 | 0.63 | 4.28 | 7.31 | 0.46 | 0.72 | 2 |
| 65 | 823655 | AA496988 | 8.96 | 2.61 | 2.67 | 3.01 | 2.02 | 4.22 | 2.85 | 1.24 | 2 |
| 68 | 246430 | N53031 | 1.50 | 0.57 | 2.63 | 3.32 | 0.54 | 7.85 | 0.69 | 0.76 | 2 |
| 67 | 491751 | AA150500 | 2.82 | 1.00 | 2.62 | 3.79 | 1.22 | 1.62 | 1.56 | 4.89 | 2 |
| 68 | 82905 | T69305 | 3.92 | 1.50 | 2.62 | 3.41 | 2.68 | 1.71 | 3.01 | 2.27 | 2 |
| 69 | 348583 | W74533 | 3.04 | 1.17 | 2.60 | 5.42 | 1.46 | 0.54 | 2.18 | 3.42 | 2 |
| 70 | 298417 | N74131 | 6.38 | 2.48 | 2.60 | 1.63 | 0.37 | 0.65 | 4.00 | 6.33 | 2 |
| 71 | 51178 | H17121 | 2.44 | 0.94 | 2.59 | 3.54 | 1.13 | 8.17 | 1.04 | 1.06 | 2 |
| 72 | 447418 | AA702335 | 4.21 | 1.63 | 2.59 | 2.51 | 0.86 | 5.37 | 3.15 | 1.03 | 2 |
| 73 | 42872 | R61883 | 0.81 | 0.32 | 2.55 | 2.17 | 1.20 | 2.58 | 3.69 | 3.11 | 2 |
| 74 | 232772 | H72722 | 39.31 | 15.73 | 2.50 | 0.44 | 0.93 | 1.09 | 5.65 | 4.38 | 2 |
| 75 | 277173 | N34316 | 19.49 | 7.91 | 2.46 | 1.63 | 3.19 | 5.67 | 1.21 | 0.61 | 2 |
| 76 | 795425 | AA453526 | 1.87 | 0.77 | 2.44 | 0.43 | 4.91 | 5.03 | 1.21 | 0.83 | 2 |
| 77 | 743868 | AA634431 | 3.37 | 1.38 | 2.44 | 0.61 | 3.72 | 2.12 | 2.49 | 3.26 | 2 |
| 78 | 40100 | R54590 | 1.16 | 0.48 | 2.41 | 3.24 | 3.10 | 1.95 | 1.76 | 1.99 | 2 |
| 79 | 202535 | H53340 | 52.42 | 21.78 | 2.41 | 0.31 | 0.53 | 0.61 | 8.54 | 4.04 | 2 |
| 80 | 267548 | N23139 | 1.37 | 0.57 | 2.41 | 0.80 | 3.38 | 5.61 | 1.13 | 1.12 | 2 |
| 81 | 740804 | AA479795 | 3.81 | 1.52 | 2.38 | 3.47 | 2.03 | 1.56 | 1.58 | 3.25 | 2 |
| 82 | 214441 | H73590 | 3.93 | 1.68 | 2.37 | 0.70 | 3.78 | 0.22 | 0.28 | 6.89 | 2 |
| 83 | 384022 | AA021586 | 3.15 | 1.33 | 2.36 | 3.36 | 1.42 | 0.76 | 2.42 | 3.86 | 2 |
| 84 | 295985 | N67039 | 0.77 | 0.33 | 2.32 | 3.94 | 1.57 | 1.42 | 3.44 | 1.24 | 2 |
| 85 | 782575 | AA447522 | 2.41 | 1.04 | 2.31 | 3.32 | 0.44 | 0.53 | 2.96 | 4.33 | 2 |
| 86 | 45852 | H08862 | 0.84 | 0.37 | 2.30 | 3.12 | 1.55 | 1.05 | 3.80 | 1.96 | 2 |
| 87 | 88560 | T67053 | 15.54 | 8.85 | 2.27 | 4.65 | 2.10 | 0.08 | 0.18 | 4.34 | 2 |
| 88 | 284383 | N52151 | 2.85 | 1.26 | 2.26 | 2.81 | 0.65 | 0.30 | 4.10 | 3.45 | 2 |
| 89 | 813151 | AA456299 | 1.57 | 0.70 | 2.23 | 4.73 | 1.49 | 3.31 | 0.74 | 0.90 | 2 |
| 90 | 813179 | AA458321 | 4.78 | 2.14 | 2.23 | 3.34 | 0.83 | 0.42 | 4.69 | 1.87 | 2 |
| 91 | 855745 | AA683981 | 28.11 | 12.67 | 2.22 | 0.76 | 5.59 | 0.11 | 0.24 | 4.40 | 2 |
| 92 | 1805539 | AA988345 | 12.27 | 5.54 | 2.22 | 0.56 | 1.01 | 0.91 | 4.70 | 3.90 | 2 |
| 93 | 144747 | R76247 | 3.07 | 1.40 | 2.20 | 3.06 | 0.82 | 2.15 | 3.63 | 1.32 | 2 |
| 94 | 50772 | H16803 | 37.52 | 17.15 | 2.19 | 3.40 | 0.78 | 0.45 | 0.51 | 5.80 | 2 |
| 95 | 214162 | H77766 | 34.05 | 15.57 | 2.19 | 0.17 | 0.51 | 0.43 | 5.78 | 4.05 | 2 |
| 96 | 247117 | N57872 | 29.18 | 13.39 | 2.16 | 0.32 | 0.52 | 0.59 | 5.39 | 4.08 | 2 |
| 97 | 1488260 | AA884926 | 6.47 | 2.98 | 2.17 | 1.03 | 4.41 | 0.25 | 0.23 | 4.96 | 2 |
| 98 | 179083 | H50086 | 0.52 | 0.24 | 2.17 | 3.28 | 0.97 | 1.63 | 1.08 | 3.89 | 2 |
| 99 | 770888 | AA434487 | 3.35 | 1.57 | 2.14 | 4.01 | 3.49 | 0.97 | 0.71 | 1.53 | 2 |
| 100 | 129585 | R18596 | 22.38 | 10.50 | 2.13 | 0.34 | 0.49 | 0.49 | 5.39 | 3.95 | 2 |
| 101 | 324815 | W49563 | 3.60 | 1.71 | 2.11 | 1.59 | 3.05 | 3.40 | 1.60 | 0.92 | 2 |
| 102 | 291097 | N72137 | 7.69 | 3.85 | 2.11 | 0.89 | 1.35 | 3.00 | 1.67 | 3.82 | 2 |
| 103 | 530814 | AA070226 | 47.09 | 22.41 | 2.10 | 3.61 | 0.50 | 0.93 | 2.03 | 3.44 | 2 |
| 104 | 1032405 | AA779457 | 1.81 | 0.86 | 2.10 | 0.76 | 0.67 | 1.22 | 3.46 | 4.38 | 2 |
| 105 | 49303 | H15877 | 0.82 | 0.30 | 2.09 | 3.99 | 0.93 | 0.84 | 1.07 | 3.80 | 2 |
| 106 | 1031701 | AA609586 | 1.73 | 0.83 | 2.07 | 3.33 | 1.92 | 3.02 | 1.08 | 1.03 | 2 |
| 107 | 530185 | AA111969 | 22.57 | 10.94 | 2.06 | 0.84 | 3.63 | 1.80 | 0.44 | 3.61 | 2 |
| 108 | 853998 | AA888897 | 19.09 | 9.27 | 2.06 | 3.08 | 2.55 | 0.49 | 0.84 | 3.34 | 2 |
| 109 | 378813 | AA683520 | 7.78 | 3.81 | 2.04 | 1.09 | 3.29 | 0.41 | 2.36 | 3.05 | 2 |
| 110 | 810899 | AA459292 | 0.02 | 0.01 | 2.01 | 0.56 | 3.70 | 1.13 | 0.40 | 4.26 | 2 |
| 111 | 297392 | N80129 | 50.71 | 25.44 | 1.99 | 0.27 | 0.58 | 0.52 | 4.51 | 4.08 | 2 |
| 112 | 471682 | AA035558 | 4.12 | 2.07 | 1.99 | 0.76 | 1.69 | 0.98 | 3.19 | 3.30 | 2 |
| 113 | 27098 | R38989 | 2.20 | 1.11 | 1.98 | 1.43 | 1.05 | 1.23 | 3.09 | 3.07 | 2 |
| 114 | 810873 | AA459197 | 2.96 | 1.50 | 1.97 | 3.13 | 1.99 | 0.68 | 0.93 | 3.11 | 2 |
| 115 | 485854 | AA040424 | 1.67 | 0.85 | 1.96 | 1.39 | 0.93 | 0.93 | 3.41 | 3.16 | 2 |
| 116 | 293292 | N64708 | 2.97 | 1.51 | 1.96 | 1.40 | 0.75 | 0.78 | 3.03 | 3.85 | 2 |
| 117 | 289337 | N92848 | 4.29 | 2.19 | 1.96 | 0.92 | 3.84 | 0.37 | 0.54 | 4.12 | 2 |
| 118 | 80948 | T70057 | 15.71 | 8.31 | 1.89 | 1.25 | 3.02 | 0.38 | 0.58 | 4.24 | 2 |
| 119 | 838500 | AA457528 | 1.44 | 0.76 | 1.89 | 0.74 | 0.90 | 0.85 | 3.49 | 3.47 | 2 |
| 120 | 343987 | W70234 | 28.35 | 15.04 | 1.89 | 0.91 | 3.19 | 1.87 | 0.41 | 3.05 | 2 |
| 121 | 258300 | N30680 | 25.74 | 13.84 | 1.86 | 0.55 | 3.33 | 4.05 | 0.75 | 0.81 | 2 |
| 122 | 824340 | AA489886 | 5.85 | 3.24 | 1.80 | 0.42 | 0.66 | 0.52 | 3.65 | 3.76 | 2 |
| 123 | 884608 | AA630008 | 37.58 | 21.21 | 1.77 | 0.69 | 1.44 | 0.21 | 3.37 | 3.15 | 2 |
| 124 | 132702 | R27004 | 48.33 | 27.34 | 1.77 | 0.26 | 0.56 | 3.72 | 3.19 | 1.11 | 2 |
| 125 | 767449 | AA418000 | 5.72 | 3.24 | 1.76 | 0.57 | 3.19 | 3.89 | 0.56 | 0.81 | 2 |
| 126 | 35804 | R48000 | 1.59 | 0.94 | 1.69 | 0.36 | 3.18 | 3.81 | 0.63 | 0.49 | 2 |
| 127 | 1031113 | AA609914 | 0.03 | 0.02 | 1.66 | 0.00 | 0.53 | 3.25 | 4.04 | 0.48 | 2 |
| 128 | 300051 | N78927 | 19.97 | 0.85 | 23.41 | 0.70 | 0.74 | 112.76 | 1.75 | 1.08 | 1 |
| 129 | 279690 | N48325 | 4.58 | 0.69 | 6.60 | 0.79 | 29.27 | 0.85 | 1.11 | 0.98 | 1 |
| 130 | 384015 | AA702640 | 8.01 | 1.23 | 6.50 | 1.41 | 0.57 | 29.22 | 0.77 | 0.54 | 1 |
| 131 | 303139 | N90806 | 2.77 | 0.50 | 5.50 | 22.37 | 1.05 | 0.71 | 2.45 | 0.91 | 1 |
| 132 | 455271 | AA677574 | 4.56 | 0.90 | 5.09 | 0.45 | 1.08 | 22.44 | 1.00 | 0.47 | 1 |
| 133 | 844703 | AA670123 | 6.75 | 1.44 | 4.69 | 0.65 | 1.51 | 20.07 | 0.72 | 0.47 | 1 |
| 134 | 841507 | AA487385 | 4.47 | 1.11 | 4.04 | 1.25 | 1.30 | 2.10 | 0.96 | 14.57 | 1 |
| 135 | 427978 | AA001834 | 1.51 | 0.38 | 3.99 | 1.46 | 0.93 | 0.99 | 1.52 | 15.05 | 1 |
| 136 | 275738 | R93176 | 1.43 | 0.36 | 3.97 | 1.32 | 1.48 | 15.25 | 0.98 | 0.83 | 1 |
| 137 | 258083 | N30328 | 1.93 | 0.49 | 3.95 | 15.18 | 1.72 | 1.03 | 0.81 | 1.02 | 1 |
| 138 | 1412481 | AA845158 | 13.18 | 3.35 | 3.94 | 18.72 | 0.22 | 0.17 | 0.30 | 0.28 | 1 |
| 139 | 838858 | AA484880 | 1.95 | 0.50 | 3.89 | 1.57 | 1.57 | 14.04 | 1.30 | 0.95 | 1 |
| 140 | 741831 | AA402874 | 11.01 | 2.97 | 3.71 | 2.12 | 2.47 | 0.42 | 11.98 | 1.56 | 1 |
| 141 | 795265 | AA454018 | 21.03 | 5.86 | 3.59 | 10.78 | 2.96 | 2.48 | 0.82 | 0.90 | 1 |
| 142 | 133130 | R28397 | 16.43 | 4.72 | 3.48 | 1.50 | 2.78 | 8.53 | 1.78 | 2.81 | 1 |
| 143 | 811581 | AA182848 | 1.54 | 0.47 | 3.25 | 2.26 | 0.92 | 10.97 | 0.92 | 1.20 | 1 |
| 144 | 812105 | AA458008 | 2.56 | 0.83 | 3.08 | 9.47 | 2.24 | 1.28 | 0.97 | 1.44 | 1 |
| 145 | 433155 | AA680138 | 17.91 | 5.90 | 3.03 | 0.97 | 1.78 | 12.03 | 0.14 | 0.25 | 1 |
| 146 | 430485 | AA680186 | 11.29 | 3.77 | 3.00 | 11.19 | 1.53 | 0.30 | 0.45 | 1.53 | 1 |
| 147 | 1637302 | A1005521 | 7.42 | 2.50 | 2.97 | 1.61 | 1.41 | 1.31 | 2.14 | 8.39 | 1 |
| 148 | 140131 | R66008 | 1.79 | 0.62 | 2.90 | 7.23 | 2.08 | 1.24 | 2.35 | 1.63 | 1 |
| 149 | 197525 | 852001 | 5.34 | 1.88 | 2.86 | 6.18 | 1.79 | 0.89 | 2.51 | 2.96 | 1 |
| 150 | 347429 | W81196 | 1.14 | 0.40 | 2.86 | 2.06 | 1.67 | 6.33 | 2.72 | 1.53 | 1 |
| 151 | 123265 | R00284 | 4.00 | 1.41 | 2.84 | 0.85 | 0.91 | 0.63 | 2.07 | 9.72 | 1 |
| 152 | 788525 | AA452113 | 5.61 | 1.98 | 2.83 | 11.85 | 0.64 | 0.89 | 0.32 | 0.46 | 1 |
| 153 | 726835 | AA398348 | 2.78 | 0.99 | 2.82 | 9.16 | 2.34 | 0.48 | 0.86 | 1.25 | 1 |
| 154 | 190325 | 829897 | 4.39 | 1.58 | 2.81 | 2.20 | 1.37 | 7.51 | 1.74 | 1.22 | 1 |
| 155 | 172785 | 820048 | 1.54 | 0.55 | 2.79 | 1.31 | 1.04 | 8.97 | 1.53 | 1.08 | 1 |
| 156 | 382419 | AA018338 | 4.81 | 1.73 | 2.77 | 1.19 | 0.72 | 9.78 | 1.54 | 0.64 | 1 |
| 157 | 188151 | H45810 | 2.40 | 0.87 | 2.77 | 7.19 | 1.65 | 1.37 | 1.53 | 2.10 | 1 |
| 158 | 47652 | H11433 | 1.43 | 0.53 | 2.73 | 2.52 | 1.92 | 4.50 | 2.72 | 1.97 | 1 |
| 159 | 825736 | AA504842 | 1.14 | 0.42 | 2.72 | 7.11 | 1.71 | 2.45 | 1.19 | 1.16 | 1 |
| 160 | 825719 | AA504834 | 1.17 | 0.43 | 2.70 | 8.04 | 2.48 | 1.38 | 0.47 | 1.15 | 1 |
| 161 | 753770 | AA408185 | 1.59 | 0.59 | 2.70 | 8.37 | 1.62 | 1.49 | 1.01 | 1.00 | 1 |
| 162 | 258242 | N30655 | 0.86 | 0.32 | 2.69 | 7.33 | 2.56 | 1.46 | 1.31 | 0.78 | 1 |
| 163 | 309893 | N94487 | 0.08 | 0.03 | 2.68 | 2.18 | 2.09 | 1.29 | 2.94 | 4.92 | 1 |
| 164 | 380213 | AA013094 | 2.09 | 0.78 | 2.68 | 1.23 | 1.08 | 1.37 | 2.45 | 7.29 | 1 |
| 165 | 416374 | W86202 | 27.30 | 10.28 | 2.68 | 2.26 | 0.82 | 6.98 | 2.34 | 0.89 | 1 |
| 166 | 259462 | N29545 | 2.91 | 1.10 | 2.64 | 8.17 | 0.60 | 1.8l | 1.38 | 1.24 | 1 |
| 167 | 1049287 | AA620757 | 1.35 | 0.51 | 2.83 | 1.18 | 1.66 | 2.31 | 6.91 | 1.09 | 1 |
| 168 | 293078 | N68719 | 3.49 | 1.34 | 2.61 | 8.78 | 0.45 | 0.72 | 0.84 | 2.26 | 1 |
| 169 | 230317 | H80853 | 1.51 | 0.58 | 2.60 | 7.04 | 1.84 | 1.86 | 0.88 | 1.37 | 1 |
| 170 | 430388 | AA680070 | 1.46 | 0.57 | 2.57 | 0.95 | 1.03 | 0.82 | 1.51 | 8.56 | 1 |
| 171 | 123354 | T99617 | 1.43 | 0.56 | 2.55 | 7.88 | 2.00 | 1.08 | 0.80 | 0.99 | 1 |
| 172 | 34129 | R44664 | 0.24 | 0.09 | 2.53 | 6.15 | 1.57 | 1.33 | 1.08 | 2.52 | 1 |
| 173 | 814001 | AA455654 | 0.47 | 0.19 | 2.50 | 2.49 | 1.73 | 6.37 | 0.60 | 1.31 | 1 |
| 174 | 509516 | AA047340 | 1.99 | 0.80 | 2.50 | 5.32 | 2.07 | 1.21 | 2.93 | 0.97 | 1 |
| 175 | 757337 | AA437099 | 6.18 | 2.48 | 2.49 | 2.25 | 2.76 | 5.79 | 0.81 | 0.84 | 1 |
| 176 | 1573251 | AA953357 | 4.61 | 1.86 | 2.48 | 2.94 | 1.78 | 1.60 | 3.69 | 2.39 | 1 |
| 177 | 1031478 | AA809218 | 2.42 | 0.98 | 2.48 | 1.39 | 8.09 | 1.08 | 0.87 | 0.98 | 1 |
| 178 | 46367 | H09959 | 5.08 | 2.05 | 2.48 | 6.85 | 0.87 | 1.34 | 1.37 | 1.95 | 1 |
| 179 | 840967 | AA486561 | 0.63 | 0.25 | 2.48 | 1.73 | 1.30 | 5.97 | 1.83 | 1.55 | 1 |
| 180 | 701819 | AA287122 | 2.53 | 1.02 | 2.47 | 2.69 | 2.05 | 2.59 | 1.74 | 3.31 | 1 |
| 181 | 685801 | AA262080 | 0.04 | 0.02 | 2.47 | 5.71 | 2.57 | 1.31 | 0.13 | 2.62 | 1 |
| 182 | 1492202 | AA875913 | 4.20 | 1.70 | 2.47 | 0.42 | 8.64 | 2.07 | 0.62 | 0.58 | 1 |
| 183 | 810328 | AA464143 | 3.11 | 1.27 | 2.46 | 2.81 | 1.79 | 2.31 | 3.29 | 2.09 | 1 |
| 184 | 128738 | R09980 | 3.62 | 1.48 | 2.45 | 1.48 | 1.49 | 5.23 | 2.65 | 1.42 | 1 |
| 185 | 28738 | R39891 | 24.84 | 10.13 | 2.45 | 5.94 | 2.27 | 2.79 | 0.58 | 0.68 | 1 |
| 186 | 277598 | N57002 | 7.63 | 3.12 | 2.45 | 2.00 | 0.74 | 1.29 | 0.68 | 7.52 | 1 |
| 187 | 1049321 | AA820783 | 0.12 | 0.05 | 2.44 | 6.15 | 2.44 | 1.71 | 0.50 | 1.41 | 1 |
| 188 | 812294 | AA455087 | 5.18 | 2.13 | 2.44 | 2.76 | 3.00 | 3.50 | 1.44 | 1.48 | 1 |
| 189 | 758657 | AA444083 | 1.98 | 0.82 | 2.43 | 6.14 | 2.83 | 1.08 | 1.02 | 1.09 | 1 |
| 190 | 375827 | AA039851 | 0.74 | 0.31 | 2.42 | 5.00 | 0.91 | 2.14 | 2.50 | 1.53 | 1 |
| 191 | 451587 | AA707086 | 2.17 | 0.90 | 2.41 | 2.98 | 3.68 | 1.47 | 1.77 | 2.16 | 1 |
| 192 | 773839 | AA431887 | 2.93 | 1.22 | 2.40 | 3.34 | 1.57 | 2.97 | 2.96 | 1.18 | 1 |
| 193 | 471725 | AA035450 | 3.23 | 1.34 | 2.40 | 1.30 | 2.63 | 5.01 | 1.45 | 1.63 | 1 |
| 194 | 239811 | H79534 | 6.57 | 2.74 | 2.40 | 8.49 | 0.66 | 1.21 | 0.73 | 0.91 | 1 |
| 195 | 38554 | R49731 | 1.45 | 0.61 | 2.40 | 1.00 | 2.75 | 5.81 | 1.20 | 1.23 | 1 |
| 196 | 291057 | N72115 | 2.08 | 0.87 | 2.39 | 6.20 | 1.47 | 0.60 | 0.99 | 2.71 | 1 |
| 197 | 83920 | T64312 | 8.32 | 3.48 | 2.39 | 1.83 | 1.62 | 4.41 | 2.58 | 1.52 | 1 |
| 198 | 388859 | AA029578 | 1.44 | 0.61 | 2.38 | 0.40 | 0.93 | 8.86 | 0.86 | 0.85 | 1 |
| 199 | 490948 | AA138818 | 9.95 | 4.19 | 2.38 | 2.79 | 1.26 | 0.38 | 1.71 | 5.74 | 1 |
| 200 | 24176 | R39325 | 8.89 | 2.90 | 2.38 | 1.31 | 1.60 | 3.50 | 2.47 | 3.00 | 1 |
| 201 | 627351 | AA190789 | 4.49 | 1.89 | 2.38 | 1.87 | 2.78 | 5.01 | 0.98 | 1.25 | 1 |
| 202 | 760299 | AA425947 | 3.73 | 1.87 | 2.37 | 6.37 | 0.77 | 0.92 | 2.21 | 1.58 | 1 |
| 203 | 858849 | AA668348 | 0.08 | 0.04 | 2.37 | 1.24 | 3.50 | 2.37 | 2.15 | 2.61 | 1 |
| 204 | 509731 | AA045698 | 28.85 | 12.22 | 2.36 | 3.84 | 1.94 | 1.22 | 2.24 | 2.57 | 1 |
| 205 | 137984 | R63085 | 1.00 | 0.42 | 2.36 | 7.58 | 0.87 | 0.59 | 1.84 | 0.90 | 1 |
| 206 | 1584628 | AA972352 | 5.99 | 2.54 | 2.36 | 1.80 | 1.04 | 1.38 | 2.27 | 5.28 | 1 |
| 207 | 49354 | H15099 | 1.57 | 0.67 | 2.35 | 3.82 | 0.92 | 1.73 | 2.46 | 2.83 | 1 |
| 208 | 280957 | N50853 | 1.46 | 0.62 | 2.35 | 4.34 | 2.38 | 1.26 | 1.96 | 1.81 | 1 |
| 209 | 348712 | W74653 | 0.19 | 0.08 | 2.34 | 1.35 | 2.45 | 2.07 | 2.15 | 3.70 | 1 |
| 210 | 273501 | N33264 | 0.10 | 0.04 | 2.32 | 2.91 | 4.49 | 1.50 | 2.63 | 0.10 | 1 |
| 211 | 25052 | R38891 | 0.68 | 0.29 | 2.32 | 7.79 | 0.96 | 1.15 | 0.70 | 1.01 | 1 |
| 212 | 1475848 | AA872006 | 1.37 | 0.59 | 2.31 | 1.54 | 1.01 | 7.24 | 0.94 | 0.84 | 1 |
| 213 | 772416 | AA405543 | 5.33 | 2.31 | 2.31 | 2.87 | 1.25 | 2.25 | 3.43 | 1.73 | 1 |
| 214 | 133341 | R27193 | 3.83 | 1.88 | 2.31 | 6.09 | 1.78 | 1.50 | 1.04 | 1.12 | 1 |
| 215 | 212784 | H69691 | 18.78 | 8.14 | 2.30 | 1.86 | 2.84 | 3.09 | 2.30 | 1.62 | 1 |
| 216 | 308013 | N91385 | 0.79 | 0.35 | 2.29 | 5.46 | 2.95 | 1.04 | 0.75 | 1.26 | 1 |
| 217 | 418833 | W88653 | 5.07 | 2.22 | 2.29 | 3.95 | 1.56 | 1.89 | 2.72 | 1.32 | 1 |
| 218 | 430336 | AA010619 | 0.05 | 0.02 | 2.27 | 1.11 | 6.23 | 0.42 | 2.16 | 1.46 | 1 |
| 219 | 271050 | N29914 | 2.05 | 0.90 | 2.27 | 0.84 | 1.34 | 0.63 | 1.63 | 6.91 | 1 |
| 220 | 488706 | AA044906 | 3.97 | 1.76 | 2.26 | 0.61 | 2.73 | 4.11 | 1.96 | 1.85 | 1 |
| 221 | 177074 | H40921 | 2.07 | 0.92 | 2.25 | 6.34 | 2.76 | 0.90 | 0.63 | 0.64 | 1 |
| 222 | 450382 | AA703652 | 1.89 | 0.84 | 2.25 | 5.62 | 1.95 | 1.44 | 1.03 | 1.21 | 1 |
| 223 | 278808 | N66572 | 0.35 | 0.15 | 2.25 | 2.17 | 1.95 | 3.50 | 1.83 | 1.80 | 1 |
| 224 | 814682 | AA481045 | 2.35 | 1.05 | 2.25 | 2.63 | 1.95 | 3.19 | 1.73 | 1.73 | 1 |
| 225 | 203003 | H54417 | 13.95 | 6.24 | 2.24 | 0.70 | 1.85 | 5.54 | 1.84 | 1.26 | 1 |
| 226 | 798480 | AA460422 | 1.56 | 0.70 | 2.23 | 7.25 | 0.71 | 0.76 | 1.23 | 1.18 | 1 |
| 227 | 431553 | AA676265 | 0.95 | 0.42 | 2.23 | 1.54 | 1.29 | 5.46 | 1.52 | 1.30 | 1 |
| 228 | 48958 | H10342 | 0.44 | 0.20 | 2.22 | 1.18 | 2.44 | 5.42 | 1.14 | 0.95 | 1 |
| 229 | 85800 | T72067 | 5.86 | 2.64 | 2.22 | 1.33 | 1.67 | 4.12 | 2.63 | 1.35 | 1 |
| 230 | 450049 | AA703398 | 27.25 | 12.29 | 2.22 | 1.45 | 4.72 | 1.88 | 2.03 | 1.03 | 1 |
| 231 | 123971 | R00884 | 8.43 | 3.81 | 2.21 | 1.06 | 2.58 | 1.20 | 1.44 | 4.78 | 1 |
| 232 | 588440 | AA148862 | 3.88 | 1.75 | 2.21 | 0.96 | 1.59 | 4.58 | 1.98 | 1.92 | 1 |
| 233 | 380437 | AA054135 | 1.62 | 0.74 | 2.20 | 3.76 | 0.89 | 2.73 | 1.67 | 1.94 | 1 |
| 234 | 852577 | AA663092 | 0.14 | 0.08 | 2.19 | 1.15 | 2.11 | 2.11 | 0.95 | 4.63 | 1 |
| 235 | 45501 | H08397 | 0.19 | 0.09 | 2.19 | 1.90 | 0.72 | 4.87 | 1.52 | 1.95 | 1 |
| 236 | 841664 | AA487560 | 2.86 | 1.30 | 2.19 | 2.71 | 1.97 | 0.79 | 2.03 | 3.44 | 1 |
| 237 | 377388 | AA055221 | 1.41 | 0.65 | 2.18 | 3.58 | 1.19 | 2.87 | 2.07 | 1.20 | 1 |
| 238 | 428377 | AA005350 | 0.92 | 0.42 | 2.18 | 0.90 | 2.13 | 4.67 | 1.89 | 1.31 | 1 |
| 239 | 46620 | H10038 | 6.81 | 3.12 | 2.18 | 5.62 | 1.46 | 1.83 | 0.67 | 1.32 | 1 |
| 240 | 296170 | W02426 | 0.64 | 0.29 | 2.17 | 0.82 | 1.27 | 0.94 | 1.66 | 6.18 | 1 |
| 241 | 795755 | AA480313 | 0.93 | 0.43 | 2.17 | 0.67 | 2.37 | 6.28 | 0.63 | 0.91 | 1 |
| 242 | 782308 | AA432253 | 0.52 | 0.24 | 2.17 | 1.76 | 0.60 | 5.05 | 1.38 | 2.06 | 1 |
| 243 | 23869 | R38179 | 0.46 | 0.21 | 2.17 | 1.13 | 1.51 | 1.93 | 3.94 | 2.32 | 1 |
| 244 | 245547 | N55167 | 6.27 | 2.90 | 2.16 | 4.21 | 1.04 | 1.14 | 2.31 | 2.13 | 1 |
| 245 | 810459 | AA457138 | 0.98 | 0.44 | 2.16 | 1.05 | 1.83 | 5.62 | 1.54 | 0.78 | 1 |
| 246 | 50922 | H19324 | 0.13 | 0.06 | 2.16 | 2.03 | 1.74 | 1.78 | 3.03 | 2.22 | 1 |
| 247 | 213804 | H72093 | 1.98 | 0.92 | 2.16 | 4.70 | 2.25 | 0.93 | 2.20 | 0.70 | 1 |
| 248 | 950710 | AA608575 | 1.16 | 0.54 | 2.15 | 2.48 | 1.64 | 1.05 | 3.66 | 1.92 | 1 |
| 249 | 491198 | AA137078 | 3.59 | 1.67 | 2.15 | 0.86 | 2.63 | 3.25 | 1.66 | 2.35 | 1 |
| 250 | 151087 | H02039 | 1.35 | 0.63 | 2.15 | 1.70 | 1.61 | 4.58 | 0.93 | 1.92 | 1 |
| 251 | 745072 | AA626275 | 1.44 | 0.67 | 2.14 | 7.11 | 1.02 | 1.03 | 0.75 | 0.82 | 1 |
| 252 | 506011 | AA708438 | 10.58 | 4.96 | 2.13 | 2.42 | 1.78 | 1.54 | 1.79 | 3.15 | 1 |
| 253 | 809881 | AA455151 | 9.38 | 4.40 | 2.13 | 1.25 | 1.42 | 2.39 | 3.13 | 2.49 | 1 |
| 254 | 49227 | H15408 | 1.21 | 0.57 | 2.13 | 1.54 | 1.12 | 2.76 | 2.02 | 3.20 | 1 |
| 255 | 590338 | AA156054 | 63.20 | 29.69 | 2.13 | 3.08 | 2.37 | 1.64 | 1.32 | 2.24 | 1 |
| 256 | 47597 | H12081 | 0.14 | 0.07 | 2.13 | 2.25 | 1.76 | 2.45 | 1.07 | 3.11 | 1 |
| 257 | 1239995 | AA706738 | 2.07 | 0.97 | 2.13 | 1.42 | 1.39 | 4.00 | 1.75 | 2.08 | 1 |
| 258 | 841621 | AA487468 | 3.48 | 1.63 | 2.13 | 5.74 | 1.33 | 1.60 | 0.93 | 1.02 | 1 |
| 259 | 884882 | AA629903 | 6.11 | 2.88 | 2.12 | 1.78 | 1.57 | 3.22 | 1.74 | 2.31 | 1 |
| 260 | 383199 | AA074049 | 0.72 | 0.34 | 2.12 | 1.00 | 1.43 | 6.87 | 0.67 | 0.64 | 1 |
| 261 | 377692 | AA058013 | 2.41 | 1.14 | 2.12 | 7.07 | 0.71 | 0.84 | 1.32 | 0.63 | 1 |
| 262 | 81320 | T60063 | 1.49 | 0.70 | 2.11 | 5.29 | 2.02 | 1.49 | 0.63 | 1.14 | 1 |
| 263 | 278242 | N63587 | 1.21 | 0.57 | 2.11 | 0.88 | 0.96 | 7.10 | 0.79 | 0.82 | 1 |
| 264 | 1558528 | AA935560 | 2.72 | 1.29 | 2.10 | 9.46 | 0.10 | 0.09 | 0.61 | 0.26 | 1 |
| 265 | 358531 | W96134 | 2.02 | 0.98 | 2.10 | 2.07 | 1.42 | 1.00 | 4.75 | 1.28 | 1 |
| 266 | 87092 | T58728 | 1.80 | 0.88 | 2.10 | 6.43 | 1.42 | 0.60 | 0.64 | 1.40 | 1 |
| 267 | 810998 | AA485352 | 3.68 | 1.78 | 2.09 | 1.07 | 1.12 | 4.37 | 2.29 | 1.62 | 1 |
| 268 | 714498 | AA292429 | 0.09 | 0.04 | 2.09 | 0.00 | 2.24 | 1.13 | 2.78 | 4.32 | 1 |
| 269 | 348119 | W72749 | 0.02 | 0.01 | 2.09 | 0.00 | 1.48 | 0.31 | 8.66 | 0.00 | 1 |
| 270 | 396098 | AA757717 | 0.83 | 0.40 | 2.09 | 1.64 | 1.33 | 2.69 | 0.97 | 3.82 | 1 |
| 271 | 433481 | AA699573 | 0.95 | 0.45 | 2.09 | 1.22 | 2.98 | 4.22 | 0.96 | 1.07 | 1 |
| 272 | 358531 | W96155 | 2.46 | 1.19 | 2.07 | 2.45 | 1.40 | 0.98 | 4.45 | 1.08 | 1 |
| 273 | 49885 | H28710 | 0.50 | 0.24 | 2.07 | 1.15 | 1.50 | 1.51 | 1.86 | 4.54 | 1 |
| 274 | 505059 | AA150918 | 2.08 | 1.01 | 2.07 | 2.74 | 1.41 | 0.80 | 2.07 | 3.33 | 1 |
| 275 | 288881 | N62403 | 1.32 | 0.84 | 2.07 | 5.29 | 1.40 | 1.61 | 0.90 | 1.12 | 1 |
| 276 | 183432 | H45447 | 0.88 | 0.41 | 2.07 | 4.85 | 1.29 | 2.17 | 0.99 | 1.04 | 1 |
| 277 | 84211 | T72915 | 5.02 | 2.43 | 2.08 | 2.24 | 2.29 | 0.55 | 4.59 | 0.85 | 1 |
| 278 | 44563 | H05445 | 0.98 | 0.47 | 2.08 | 4.16 | 1.53 | 0.71 | 1.33 | 2.59 | 1 |
| 279 | 134783 | R31701 | 0.49 | 0.24 | 2.06 | 4.62 | 2.76 | 1.04 | 0.75 | 1.15 | 1 |
| 280 | 759948 | AA424045 | 3.57 | 1.73 | 2.06 | 8.30 | 0.98 | 0.67 | 0.68 | 1.68 | 1 |
| 281 | 378481 | AA775816 | 10.99 | 5.34 | 2.08 | 5.11 | 1.89 | 0.55 | 1.58 | 1.17 | 1 |
| 282 | 771274 | AA443602 | 0.08 | 0.04 | 2.06 | 1.39 | 4.87 | 1.14 | 1.78 | 1.11 | 1 |
| 283 | 435036 | AA700054 | 5.18 | 2.52 | 2.05 | 1.18 | 4.24 | 1.94 | 0.78 | 2.13 | 1 |
| 284 | 448885 | AA777670 | 29.68 | 14.49 | 2.05 | 4.21 | 1.91 | 2.32 | 0.90 | 0.90 | 1 |
| 285 | 454589 | AA677050 | 0.53 | 0.26 | 2.05 | 0.98 | 1.20 | 5.31 | 1.24 | 1.51 | 1 |
| 286 | 360684 | AA015959 | 1.08 | 0.53 | 2.05 | 2.47 | 1.80 | 3.05 | 1.12 | 1.80 | 1 |
| 287 | 1603583 | AA998131 | 15.75 | 7.71 | 2.04 | 2.88 | 1.58 | 0.31 | 1.64 | 3.81 | 1 |
| 288 | 162199 | H26426 | 2.86 | 1.40 | 2.04 | 1.78 | 0.65 | 1.03 | 5.35 | 1.40 | 1 |
| 289 | 68988 | T54164 | 1.22 | 0.60 | 2.04 | 2.54 | 4.68 | 1.98 | 0.42 | 0.55 | 1 |
| 290 | 80484 | T84469 | 130.51 | 64.19 | 2.03 | 0.73 | 2.37 | 1.40 | 2.47 | 3.19 | 1 |
| 291 | 38691 | R51535 | 1.32 | 0.65 | 2.03 | 4.04 | 1.44 | 2.18 | 1.40 | 1.09 | 1 |
| 292 | 123225 | T99852 | 0.57 | 0.28 | 2.03 | 3.43 | 1.10 | 1.40 | 1.57 | 2.65 | 1 |
| 293 | 798313 | AA481311 | 0.77 | 0.38 | 2.03 | 0.68 | 2.47 | 4.79 | 1.19 | 1.00 | 1 |
| 294 | 811827 | AA463635 | 3.88 | 1.82 | 2.03 | 2.57 | 3.34 | 1.13 | 1.05 | 2.05 | 1 |
| 295 | 489079 | AA057156 | 0.87 | 0.43 | 2.03 | 4.75 | 1.83 | 1.18 | 1.43 | 0.95 | 1 |
| 296 | 281659 | N48050 | 1.97 | 0.97 | 2.02 | 6.75 | 1.03 | 0.68 | 0.83 | 0.83 | 1 |
| 297 | 241821 | H93381 | 3.86 | 1.91 | 2.02 | 1.57 | 2.36 | 1.17 | 3.90 | 1.12 | 1 |
| 298 | 149373 | H04382 | 0.81 | 0.40 | 2.02 | 1.63 | 1.24 | 1.27 | 2.54 | 3.42 | 1 |
| 299 | 76988 | T61050 | 1.24 | 0.62 | 2.02 | 3.84 | 1.07 | 0.86 | 1.51 | 2.81 | 1 |
| 300 | 388039 | AA071503 | 0.19 | 0.10 | 2.02 | 4.94 | 1.43 | 1.29 | 0.60 | 1.83 | 1 |
| 301 | 827152 | AA521247 | 6.91 | 3.42 | 2.02 | 3.59 | 2.10 | 0.88 | 1.34 | 2.19 | 1 |
| 302 | 45623 | H08424 | 2.14 | 1.06 | 2.02 | 3.32 | 1.03 | 2.08 | 1.84 | 1.81 | 1 |
| 303 | 785818 | AA449762 | 0.07 | 0.03 | 2.02 | 3.95 | 1.65 | 1.67 | 1.36 | 1.46 | 1 |
| 304 | 209025 | H83416 | 10.60 | 5.26 | 2.02 | 5.41 | 1.88 | 0.48 | 0.82 | 1.49 | 1 |
| 305 | 80221 | T64223 | 3.49 | 1.73 | 2.01 | 2.79 | 1.82 | 0.49 | 1.70 | 3.27 | 1 |
| 306 | 242081 | H93335 | 2.16 | 1.07 | 2.01 | 5.85 | 1.64 | 0.95 | 0.77 | 0.85 | 1 |
| 307 | 1831807 | A1004169 | 5.46 | 2.71 | 2.01 | 1.57 | 0.75 | 1.76 | 4.12 | 1.86 | 1 |
| 308 | 508016 | AA708440 | 9.52 | 4.73 | 2.01 | 2.43 | 1.27 | 3.33 | 1.54 | 1.49 | 1 |
| 309 | 190732 | H38650 | 4.69 | 2.33 | 2.01 | 2.81 | 2.08 | 0.82 | 0.81 | 3.72 | 1 |
| 310 | 322537 | W15263 | 1.85 | 0.92 | 2.01 | 0.81 | 1.81 | 5.26 | 1.42 | 0.73 | 1 |
| 311 | 249526 | H84369 | 1.24 | 0.82 | 2.01 | 0.99 | 1.77 | 5.61 | 0.87 | 0.80 | 1 |
| 312 | 279905 | N38860 | 1.54 | 0.77 | 2.00 | 5.19 | 1.05 | 1.08 | 1.56 | 1.13 | 1 |
| 313 | 858188 | AA633866 | 0.92 | 0.48 | 2.00 | 1.73 | 3.32 | 2.15 | 1.52 | 1.31 | 1 |
| 314 | 856420 | AA630771 | 22.32 | 11.14 | 2.00 | 3.60 | 1.35 | 2.39 | 1.03 | 1.68 | 1 |
| 315 | 249603 | H84871 | 7.73 | 3.88 | 2.00 | 1.47 | 1.87 | 4.42 | 1.41 | 0.84 | 1 |
| 316 | 49333 | H15417 | 0.09 | 0.04 | 1.99 | 2.04 | 1.11 | 1.08 | 4.19 | 1.54 | 1 |
| 317 | 233464 | H77297 | 20.24 | 10.17 | 1.99 | 2.98 | 2.62 | 0.25 | 0.75 | 3.34 | 1 |
| 318 | 758388 | AA404293 | 0.70 | 0.35 | 1.99 | 1.75 | 1.44 | 5.14 | 0.64 | 0.97 | 1 |
| 319 | 838899 | AA484935 | 0.72 | 0.38 | 1.99 | 1.38 | 2.31 | 3.80 | 1.48 | 0.95 | 1 |
| 320 | 382628 | AA017133 | 2.42 | 1.22 | 1.98 | 1.53 | 1.96 | 3.89 | 1.53 | 1.01 | 1 |
| 321 | 731031 | AA421270 | 5.97 | 3.01 | 1.98 | 3.29 | 1.42 | 2.54 | 1.52 | 1.14 | 1 |
| 322 | 83999 | T70892 | 4.15 | 2.09 | 1.98 | 3.41 | 1.82 | 1.29 | 1.54 | 1.84 | 1 |
| 323 | 418558 | W86987 | 0.95 | 0.48 | 1.98 | 1.13 | 1.17 | 6.33 | 0.53 | 0.73 | 1 |
| 324 | 788334 | AA453015 | 6.49 | 3.29 | 1.97 | 6.42 | 1.11 | 0.70 | 0.61 | 1.03 | 1 |
| 325 | 183641 | H14057 | 2.99 | 1.52 | 1.97 | 5.23 | 1.98 | 0.99 | 0.73 | 0.93 | 1 |
| 326 | 307053 | N89876 | 4.57 | 2.32 | 1.97 | 1.57 | 1.48 | 0.44 | 2.22 | 4.11 | 1 |
| 327 | 842918 | AA486435 | 8.31 | 3.21 | 1.97 | 1.03 | 0.85 | 1.46 | 1.50 | 4.99 | 1 |
| 328 | 29954 | R42520 | 0.54 | 0.28 | 1.96 | 1.44 | 2.60 | 3.71 | 0.65 | 1.41 | 1 |
| 329 | 183337 | H42879 | 12.08 | 8.16 | 1.96 | 3.56 | 0.88 | 2.17 | 0.81 | 2.39 | 1 |
| 330 | 200741 | R98827 | 4.83 | 2.46 | 1.96 | 1.81 | 2.73 | 0.50 | 0.70 | 4.07 | 1 |
| 331 | 770837 | AA427740 | 2.08 | 1.08 | 1.96 | 1.26 | 1.04 | 3.33 | 2.02 | 2.14 | 1 |
| 332 | 382424 | AA018480 | 2.42 | 1.24 | 1.96 | 0.90 | 0.78 | 6.62 | 0.80 | 0.68 | 1 |
| 333 | 771258 | AA443849 | 0.02 | 0.01 | 1.95 | 1.71 | 4.80 | 2.40 | 0.45 | 0.60 | 1 |
| 334 | 138474 | R34331 | 2.31 | 1.19 | 1.95 | 1.80 | 0.96 | 4.93 | 1.19 | 0.88 | 1 |
| 335 | 795262 | AA453997 | 4.82 | 2.47 | 1.95 | 0.98 | 0.95 | 0.85 | 4.12 | 2.84 | 1 |
| 336 | 1602120 | AA982236 | 13.82 | 7.09 | 1.95 | 3.17 | 1.53 | 0.94 | 1.29 | 2.81 | 1 |
| 337 | 52303 | H23137 | 0.85 | 0.43 | 1.94 | 3.19 | 1.76 | 1.70 | 1.70 | 1.37 | 1 |
| 338 | 385515 | AA009809 | 4.32 | 2.22 | 1.94 | 2.17 | 1.45 | 0.41 | 1.33 | 4.37 | 1 |
| 339 | 526587 | AA128407 | 2.02 | 1.04 | 1.94 | 0.86 | 1.59 | 3.88 | 1.50 | 1.89 | 1 |
| 340 | 377481 | AA055835 | 9.94 | 5.12 | 1.94 | 2.64 | 1.98 | 0.39 | 1.70 | 3.02 | 1 |
| 341 | 413150 | AA707872 | 0.64 | 0.33 | 1.94 | 5.51 | 1.12 | 0.85 | 0.96 | 1.26 | 1 |
| 342 | 429148 | AA004835 | 2.19 | 1.13 | 1.94 | 2.21 | 1.49 | 3.47 | 1.52 | 1.00 | 1 |
| 343 | 80228 | T84218 | 0.22 | 0.11 | 1.94 | 1.56 | 3.44 | 2.39 | 0.93 | 1.38 | 1 |
| 344 | 415833 | W88653 | 4.19 | 2.17 | 1.93 | 3.68 | 0.57 | 1.27 | 2.79 | 1.36 | 1 |
| 345 | 132954 | R24451 | 1.74 | 0.90 | 1.93 | 5.07 | 2.15 | 0.93 | 0.71 | 0.81 | 1 |
| 346 | 450050 | AA703387 | 8.72 | 4.52 | 1.93 | 0.98 | 0.93 | 1.58 | 2.82 | 3.35 | 1 |
| 347 | 548693 | AA125825 | 0.16 | 0.08 | 1.93 | 3.4.4 | 1.81 | 1.84 | 0.93 | 1.63 | 1 |
| 348 | 50877 | H18423 | 34.44 | 17.84 | 1.93 | 4.07 | 2.89 | 0.09 | 0.14 | 2.48 | 1 |
| 349 | 449257 | AA777700 | 0.12 | 0.08 | 1.93 | 1.04 | 1.53 | 3.68 | 1.22 | 2.18 | 1 |
| 350 | 588108 | AA121836 | 8.88 | 4.49 | 1.93 | 1.78 | 3.04 | 2.55 | 0.79 | 1.50 | 1 |
| 351 | 127193 | R08268 | 1.24 | 0.84 | 1.93 | 2.42 | 1.15 | 1.62 | 0.91 | 3.54 | 1 |
| 352 | 308829 | N91914 | 3.05 | 1.58 | 1.93 | 6.84 | 0.52 | 0.59 | 0.82 | 0.85 | 1 |
| 353 | 192589 | H41489 | 3.83 | 1.99 | 1.93 | 0.94 | 0.71 | 5.07 | 1.97 | 0.94 | 1 |
| 354 | 1468788 | AA883735 | 1.57 | 0.81 | 1.93 | 1.90 | 1.30 | 1.58 | 1.56 | 3.28 | I |
| 355 | 1573305 | AA953973 | 2.79 | 1.45 | 1.92 | 5.76 | 0.82 | 0.58 | 1.25 | 1.21 | 1 |
| 356 | 757265 | AA426113 | 1.80 | 0.94 | 1.92 | 3.51 | 1.16 | 2.59 | 1.40 | 0.88 | 1 |
| 357 | 687687 | AA235370 | 1.53 | 0.80 | 1.92 | 3.37 | 0.94 | 0.99 | 2.30 | 2.04 | 1 |
| 358 | 878281 | AA870286 | 7.48 | 3.89 | 1.92 | 3.56 | 0.78 | 0.41 | 2.41 | 2.47 | 1 |
| 359 | 589382 | AA130017 | 0.09 | 0.05 | 1.92 | 2.33 | 3.56 | 1.34 | 1.36 | 1.02 | 1 |
| 360 | 796268 | AA480827 | 0.35 | 0.18 | 1.92 | 3.35 | 1.77 | 1.62 | 1.36 | 1.51 | 1 |
| 361 | 859654 | AA686418 | 0.98 | 0.51 | 1.92 | 0.94 | 0.94 | 3.12 | 1.97 | 2.63 | 1 |
| 362 | 488301 | AA085748 | 11.69 | 6.09 | 1.92 | 0.85 | 0.97 | 1.90 | 3.51 | 2.37 | 1 |
| 363 | 61412 | T40905 | 0.82 | 0.43 | 1.92 | 5.44 | 1.24 | 1.01 | 0.78 | 1.12 | 1 |
| 364 | 45877 | H08582 | 1.01 | 0.53 | 1.92 | 2.29 | 3.06 | 0.67 | 1.75 | 1.81 | 1 |
| 365 | 841615 | AA487485 | 16.66 | 8.71 | 1.91 | 1.90 | 1.31 | 0.42 | 2.10 | 3.83 | 1 |
| 366 | 281072 | N50912 | 12.38 | 6.47 | 1.91 | 3.62 | 1.55 | 1.94 | 1.15 | 1.30 | 1 |
| 367 | 308068 | N91003 | 5.76 | 3.02 | 1.91 | 3.01 | 1.58 | 1.41 | 1.15 | 2.43 | 1 |
| 368 | 510576 | AA055788 | 176.33 | 92.35 | 1.91 | 3.70 | 2.54 | 1.97 | 0.68 | 0.65 | 1 |
| 369 | 431288 | AA682627 | 22.04 | 11.54 | 1.91 | 1.77 | 2.64 | 0.76 | 0.93 | 3.44 | 1 |
| 370 | 214809 | H74086 | 1.22 | 0.84 | 1.90 | 2.47 | 3.82 | 1.07 | 0.57 | 1.59 | 1 |
| 371 | 269425 | N26171 | 1.45 | 0.76 | 1.90 | 0.71 | 0.81 | 6.72 | 0.80 | 0.46 | 1 |
| 372 | 592243 | AA155895 | 4.11 | 2.17 | 1.90 | 3.33 | 1.84 | 1.17 | 1.48 | 1.87 | 1 |
| 373 | 1555451 | AA975556 | 2.52 | 1.33 | 1.90 | 1.16 | 1.16 | 3.09 | 2.42 | 1.65 | 1 |
| 374 | 292088 | N73301 | 2.84 | 1.50 | 1.89 | 2.02 | 1.49 | 3.16 | 1.31 | 1.49 | 1 |
| 375 | 825358 | AA504492 | 2.77 | 1.48 | 1.89 | 4.59 | 1.00 | 1.86 | 0.87 | 1.14 | 1 |
| 376 | 453768 | AA776434 | 0.50 | 0.27 | 1.89 | 1.02 | 1.17 | 5.41 | 1.04 | 0.81 | 1 |
| 377 | 292633 | N68576 | 0.12 | 0.07 | 1.89 | 1.69 | 3.23 | 1.87 | 1.50 | 1.16 | 1 |
| 378 | 48358 | H09936 | 3.08 | 1.63 | 1.89 | 4.90 | 1.80 | 0.86 | 0.87 | 1.00 | 1 |
| 379 | 727792 | AA393408 | 1.51 | 0.80 | 1.89 | 5.55 | 1.18 | 0.66 | 0.73 | 1.30 | 1 |
| 380 | 727184 | AA398922 | 11.03 | 5.86 | 1.88 | 0.93 | 1.83 | 3.74 | 1.54 | 1.37 | 1 |
| 381 | 39933 | R53983 | 0.76 | 0.40 | 1.88 | 1.69 | 1.16 | 0.41 | 0.47 | 5.67 | 1 |
| 382 | 290235 | N64391 | 1.51 | 0.80 | 1.88 | 3.06 | 1.13 | 1.88 | 1.44 | 1.90 | 1 |
| 383 | 125134 | R05416 | 0.04 | 0.02 | 1.88 | 3.00 | 1.42 | 1.28 | 0.80 | 2.89 | 1 |
| 384 | 47630 | H11376 | 0.98 | 0.52 | 1.88 | 0.72 | 1.23 | 5.60 | 1.05 | 0.78 | 1 |
| 385 | 511832 | AA088517 | 2.26 | 1.21 | 1.87 | 4.39 | 2.04 | 0.91 | 0.75 | 1.26 | 1 |
| 386 | 398045 | AA757659 | 3.28 | 1.75 | 1.87 | 1.72 | 1.69 | 3.24 | 1.02 | 1.68 | 1 |
| 387 | 701272 | AA288814 | 0.58 | 0.30 | 1.87 | 3.46 | 1.60 | 1.77 | 1.09 | 1.44 | 1 |
| 388 | 195127 | R91220 | 0.92 | 0.49 | 1.87 | 5.34 | 0.66 | 0.70 | 1.07 | 1.58 | 1 |
| 389 | 1471841 | AA873355 | 61.84 | 33.03 | 1.87 | 1.00 | 1.27 | 5.59 | 0.92 | 0.54 | 1 |
| 390 | 878182 | AA775447 | 22.84 | 12.25 | 1.86 | 2.37 | 0.63 | 0.31 | 3.50 | 2.52 | 1 |
| 391 | 307680 | N92901 | 1.88 | 1.01 | 1.86 | 5.12 | 1.28 | 0.88 | 1.12 | 0.93 | 1 |
| 392 | 1572198 | AA931725 | 3.38 | 1.81 | 1.86 | 1.18 | 0.89 | 4.59 | 1.52 | 1.14 | 1 |
| 393 | 745570 | AA626315 | 4.89 | 2.63 | 1.86 | 3.17 | 1.07 | 0.72 | 1.93 | 2.41 | 1 |
| 394 | 202168 | 852381 | 0.02 | 0.01 | 1.88 | 1.16 | 3.75 | 0.99 | 2.02 | 1.36 | 1 |
| 395 | 32578 | R43535 | 0.73 | 0.39 | 1.85 | 2.40 | 0.65 | 1.54 | 3.32 | 1.38 | 1 |
| 396 | 412949 | AA707840 | 1.72 | 0.93 | 1.85 | 1.70 | 1.31 | 3.01 | 0.97 | 2.27 | 1 |
| 397 | 133358 | R26859 | 1.06 | 0.57 | 1.85 | 4.35 | 1.38 | 1.58 | 0.80 | 1.16 | 1 |
| 398 | 450598 | AA704587 | 2.38 | 1.27 | 1.85 | 0.78 | 1.05 | 5.01 | 1.16 | 1.25 | 1 |
| 399 | 1493252 | AA886870 | 28.41 | 15.36 | 1.85 | 1.91 | 1.12 | 0.48 | 2.54 | 3.19 | 1 |
| 400 | 51916 | H22563 | 1.81 | 0.87 | 1.85 | 1.48 | 1.17 | 4.17 | 1.45 | 0.97 | 1 |
| 401 | 824573 | AA490987 | 0.21 | 0.12 | 1.85 | 1.40 | 1.01 | 1.51 | 0.91 | 4.40 | 1 |
| 402 | 172517 | H19826 | 2.35 | 1.27 | 1.85 | 1.33 | 1.25 | 4.54 | 1.16 | 0.95 | 1 |
| 403 | 433287 | AA699714 | 1.19 | 0.64 | 1.84 | 1.02 | 0.84 | 4.98 | 1.52 | 0.86 | 1 |
| 404 | 796876 | AA483188 | 2.71 | 1.47 | 1.84 | 3.24 | 1.50 | 0.99 | 2.00 | 1.48 | 1 |
| 405 | 321359 | W32408 | 4.84 | 2.52 | 1.84 | 2.16 | 3.33 | 1.00 | 0.88 | 1.85 | 1 |
| 406 | 147050 | R80217 | 1.47 | 0.80 | 1.84 | 1.85 | 0.92 | 4.60 | 1.26 | 0.59 | 1 |
| 407 | 825848 | AA505045 | 1.03 | 0.58 | 1.84 | 1.88 | 1.72 | 3.77 | 0.80 | 1.03 | 1 |
| 408 | 50043 | H17698 | 0.47 | 0.25 | 1.84 | 4.95 | 1.17 | 1.02 | 0.89 | 1.17 | 1 |
| 409 | 754601 | AA411387 | 0.85 | 0.46 | 1.84 | 3.56 | 2.24 | 0.86 | 1.22 | 1.31 | 1 |
| 410 | 774078 | AA441933 | 1.43 | 0.78 | 1.83 | 0.62 | 1.21 | 3.18 | 1.72 | 2.44 | 1 |
| 411 | 1408710 | AA868515 | 0.21 | 0.11 | 1.83 | 0.43 | 1.10 | 1.42 | 4.47 | 1.73 | 1 |
| 412 | 1031278 | AA609049 | 2.12 | 1.16 | 1.83 | 2.49 | 3.79 | 1.68 | 0.57 | 0.63 | 1 |
| 413 | 51320 | 820547 | 1.30 | 0.71 | 1.83 | 3.52 | 1.22 | 1.89 | 1.67 | 0.84 | 1 |
| 414 | 489706 | AA099582 | 2.00 . | 1.09 | 1.83 | 1.86 | 1.26 | 3.05 | 1.98 | 0.98 | 1 |
| 415 | 363088 | AA019482 | 3.46 | 1.89 | 1.83 | 1.12 | 1.30 | 2.12 | 3.16 | 1.44 | 1 |
| 416 | 47169 | H10761 | 0.56 | 0.31 | 1.82 | 3.69 | 1.98 | 1.07 | 1.02 | 1.38 | 1 |
| 417 | 153541 | R48320 | 0.06 | 0.03 | 1.82 | 1.10 | 2.28 | 4.94 | 0.80 | 0.00 | 1 |
| 418 | 392399 | AA707935 | 0.90 | 0.49 | 1.82 | 1.33 | 1.40 | 4.13 | 1.06 | 1.19 | 1 |
| 419 | 489553 | AA098898 | 1.45 | 0.80 | 1.82 | 1.04 | 1.22 | 3.33 | 1.71 | 1.81 | 1 |
| 420 | 277848 | N47604 | 3.01 | 1.66 | 1.82 | 1.89 | 1.05 | 3.09 | 1.68 | 1.37 | 1 |
| 421 | 878544 | AA775863 | 68.74 | 37.83 | 1.82 | 1.27 | 1.84 | 4.14 | 0.88 | 0.95 | 1 |
| 422 | 1476019 | AA872454 | 4.49 | 2.47 | 1.82 | 3.36 | 1.50 | 0.68 | 1.21 | 2.35 | 1 |
| 423 | 845259 | AA199881 | 0.17 | 0.09 | 1.82 | 1.15 | 1.76 | 3.28 | 1.38 | 1.54 | 1 |
| 424 | 262060 | H99075 | 1.58 | 0.87 | 1.81 | 0.80 | 1.91 | 4.97 | 0.58 | 0.81 | 1 |
| 425 | 812244 | AA455042 | 13.83 | 7.64 | 1.81 | 1.59 | 2.25 | 3.19 | 1.01 | 1.02 | 1 |
| 426 | 1555427 | AA975209 | 16.82 | 9.31 | 1.81 | 1.64 | 0.94 | 2.29 | 3.04 | 1.11 | 1 |
| 427 | 809620 | AA458491 | 0.46 | 0.26 | 1.81 | 0.96 | 1.26 | 4.08 | 1.73 | 0.99 | 1 |
| 428 | 611412 | AA180204 | 2.43 | 1.35 | 1.80 | 1.76 | 0.98 | 1.99 | 3.31 | 0.97 | 1 |
| 429 | 888838 | AA775223 | 4.62 | 2.58 | 1.80 | 5.26 | 0.24 | 0.30 | 1.21 | 2.01 | 1 |
| 430 | 826072 | AA521394 | 1.72 | 0.95 | 1.80 | 2.99 | 0.98 | 3.28 | 0.56 | 1.20 | 1 |
| 431 | 1558842 | AA976544 | 27.62 | 15.33 | 1.80 | 0.99 | 1.43 | 3.14 | 2.21 | 1.24 | 1 |
| 432 | 200395 | R97226 | 1.08 | 0.60 | 1.80 | 3.20 | 1.03 | 1.11 | 0.73 | 2.94 | 1 |
| 433 | 153817 | R48833 | 2.51 | 1.39 | 1.80 | 2.30 | 1.35 | 3.59 | 1.18 | 0.56 | 1 |
| 434 | 481727 | AA682293 | 0.70 | 0.39 | 1.80 | 0.59 | 0.93 | 0.79 | 1.28 | 5.40 | 1 |
| 435 | 701112 | AA287404 | 9.86 | 5.49 | 1.80 | 3.66 | 0.77 | 1.37 | 1.79 | 1.39 | 1 |
| 436 | 1580342 | AA969184 | 0.92 | 0.51 | 1.79 | 1.57 | 1.16 | 4.06 | 1.08 | 1.09 | 1 |
| 437 | 48353 | H09747 | 0.80 | 0.45 | 1.79 | 0.96 | 1.44 | 1.95 | 1.05 | 3.55 | 1 |
| 438 | 259884 | N32904 | 1.13 | 0.63 | 1.79 | 1.57 | 1.53 | 3.63 | 1.20 | 1.01 | 1 |
| 439 | 1031598 | AA609474 | 25.27 | 14.15 | 1.79 | 3.23 | 1.78 | 2.16 | 0.80 | 0.98 | 1 |
| 440 | 271662 | N35067 | 33.23 | 18.60 | 1.79 | 1.35 | 0.92 | 3.73 | 1.73 | 1.20 | 1 |
| 441 | 51773 | H23211 | 15.09 | 8.45 | 1.79 | 3.48 | 1.75 | 0.81 | 1.17 | 1.73 | 1 |
| 442 | 108864 | T78909 | 18.88 | 10.58 | 1.78 | 0.58 | 1.54 | 3.49 | 1.75 | 1.56 | 1 |
| 443 | 757873 | AA442853 | 7.87 | 4.41 | 1.78 | 1.42 | 1.72 | 1.06 | 4.23 | 0.50 | 1 |
| 444 | 815772 | AA485140 | 2.65 | 1.49 | 1.78 | 2.15 | 1.90 | 3.02 | 0.94 | 0.89 | 1 |
| 445 | 786805 | AA478479 | 1.01 | 0.57 | 1.78 | 1.44 | 4.45 | 0.80 | 1.41 | 0.80 | 1 |
| 446 | 245330 | N54596 | 1.27 | 0.72 | 1.78 | 3.75 | 1.39 | 1.60 | 1.28 | 0.89 | 1 |
| 447 | 731272 | AA420993 | 7.07 | 3.98 | 1.78 | 3.50 | 0.87 | 1.30 | 1.65 | 1.57 | 1 |
| 448 | 1609038 | A1000502 | 14.33 | 0.09 | 1.77 | 0.74 | 1.40 | 0.84 | 3.22 | 2.65 | 1 |
| 449 | 451511 | AA707336 | 0.88 | 0.38 | 1.77 | 1.20 | 4.71 | 1.29 | 0.89 | 0.77 | 1 |
| 450 | 39833 | R53455 | 10.53 | 5.95 | 1.77 | 1.75 | 1.83 | 0.21 | 1.30 | 3.76 | 1 |
| 451 | 21994 | T88320 | 2.73 | 1.54 | 1.77 | 1.17 | 1.91 | 0.38 | 1.95 | 3.43 | 1 |
| 452 | 121948 | T97762 | 9.91 | 5.60 | 1.77 | 0.47 | 1.82 | 1.12 | 1.02 | 4.42 | 1 |
| 453 | 343780 | W89271 | 5.57 | 3.15 | 1.77 | 1.32 | 1.12 | 1.43 | 1.91 | 3.08 | 1 |
| 454 | 51103 | H19217 | 0.37 | 0.21 | 1.77 | 1.90 | 0.85 | 4.47 | 0.94 | 0.65 | 1 |
| 455 | 281938 | N51069 | 1.68 | 0.95 | 1.77 | 3.44 | 1.64 | 1.71 | 0.80 | 1.23 | 1 |
| 456 | 562664 | AA086204 | 1.47 | 0.83 | 1.76 | 1.18 | 1.98 | 3.74 | 0.91 | 1.01 | 1 |
| 457 | 248288 | N78092 | 1.45 | 0.82 | 1.76 | 3.05 | 1.85 | 1.68 | 1.22 | 1.01 | 1 |
| 458 | 788180 | AA453310 | 118.87 | 67.49 | 1.76 | 2.31 | 2.20 | 3.20 | 0.56 | 0.53 | 1 |
| 459 | 449033 | AA777394 | 0.71 | 0.40 | 1.76 | 2.13 | 1.04 | 0.83 | 3.54 | 1.26 | 1 |
| 460 | 878231 | AA775774 | 5.16 | 2.93 | 1.76 | 1.47 | 1.07 | 3.15 | 2.46 | 0.63 | 1 |
| 461 | 138294 | R58854 | 3.84 | 2.19 | 1.76 | 1.02 | 1.15 | 3.32 | 1.74 | 1.58 | 1 |
| 462 | 295843 | N68957 | 4.79 | 2.73 | 1.76 | 0.55 | 0.61 | 0.35 | 1.52 | 5.76 | 1 |
| 463 | 798287 | AA460849 | 0.98 | 0.55 | 1.76 | 1.16 | 0.92 | 3.68 | 1.94 | 1.07 | 1 |
| 464 | 450612 | AA682452 | 1.00 | 0.57 | 1.75 | 1.15 | 1.61 | 4.08 | 1.37 | 0.56 | 1 |
| 465 | 884539 | AA629804 | 0.19 | 0.11 | 1.75 | 1.41 | 1.45 | 3.15 | 1.71 | 1.05 | 1 |
| 466 | 504791 | AA152347 | 20.33 | 11.60 | 1.75 | 0.95 | 1.89 | 0.27 | 2.07 | 3.58 | 1 |
| 467 | 281442 | N47902 | 4.86 | 2.77 | 1.75 | 1.78 | 1.18 | 1.01 | 3.28 | 1.52 | 1 |
| 468 | 325001 | W49583 | 1.56 | 0.89 | 1.75 | 1.28 | 1.74 | 4.25 | 0.81 | 0.68 | 1 |
| 469 | 489594 | AA099523 | 2.16 | 1.23 | 1.75 | 3.54 | 0.90 | 1.41 | 1.65 | 1.26 | 1 |
| 470 | 417777 | W88737 | 1.97 | 1.12 | 1.75 | 4.98 | 1.15 | 0.58 | 0.80 | 1.23 | 1 |
| 471 | 277266 | N34395 | 2.46 | 1.41 | 1.75 | 1.52 | 1.38 | 3.06 | 1.93 | 0.85 | 1 |
| 472 | 814341 | AA459123 | 1.84 | 1.06 | 1.75 | 2.31 | 1.29 | 0.32 | 0.72 | 4.09 | 1 |
| 473 | 276871 | N39426 | 1.00 | 0.57 | 1.75 | 3.28 | 0.79 | 2.59 | 1.10 | 0.97 | 1 |
| 474 | 713129 | AA283007 | 1.69 | 0.97 | 1.74 | 2.26 | 1.04 | 0.48 | 1.14 | 3.80 | 1 |
| 475 | 208288 | H58702 | 1.59 | 0.91 | 1.74 | 0.78 | 1.31 | 6.00 | 0.32 | 0.30 | 1 |
| 476 | 703577 | AA278757 | 1.58 | 0.91 | 1.74 | 3.59 | 2.66 | 0.74 | 0.92 | 0.80 | 1 |
| 477 | 757244 | AA426025 | 14.29 | 8.20 | 1.74 | 1.16 | 2.01 | 3.61 | 0.76 | 1.17 | 1 |
| 478 | 646857 | AA205413 | 0.12 | 0.07 | 1.74 | 1.33 | 1.89 | 3.01 | 1.38 | 1.10 | 1 |
| 479 | 809719 | AA455497 | 1.58 | 0.90 | 1.74 | 3.53 | 0.89 | 0.89 | 2.12 | 1.26 | 1 |
| 480 | 1601852 | AA989210 | 2.60 | 1.50 | 1.74 | 1.45 | 1.29 | 1.07 | 1.83 | 3.04 | 1 |
| 481 | 264166 | N20482 | 1.05 | 0.61 | 1.73 | 1.00 | 2.01 | 4.19 | 0.50 | 0.96 | 1 |
| 482 | 235909 | H52247 | 0.98 | 0.57 | 1.73 | 1.27 | 1.15 | 3.01 | 1.54 | 1.70 | 1 |
| 483 | 809374 | AA456569 | 2.00 | 1.16 | 1.73 | 1.55 | 1.06 | 3.80 | 1.40 | 0.85 | 1 |
| 484 | 753907 | AA479351 | 2.48 | 1.42 | 1.73 | 1.13 | 2.12 | 3.99 | 0.71 | 0.71 | 1 |
| 485 | 755299 | AA498359 | 1.93 | 1.12 | 1.73 | 0.98 | 2.29 | 0.96 | 3.61 | 0.81 | 1 |
| 486 | 878411 | AA670356 | 2.30 | 1.33 | 1.73 | 1.85 | 0.98 | 1.37 | 3.32 | 1.14 | 1 |
| 487 | 609935 | AA169154 | 0.09 | 0.05 | 1.73 | 0.66 | 1.68 | 1.38 | 3.49 | 1.43 | 1 |
| 488 | 700603 | AA291137 | 4.08 | 2.37 | 1.72 | 1.21 | 1.27 | 3.64 | 1.30 | 1.19 | 1 |
| 489 | 898098 | AA598798 | 7.78 | 4.51 | 1.72 | 3.95 | 1.00 | 0.84 | 1.59 | 1.24 | 1 |
| 490 | 782576 | AA447514 | 0.52 | 0.30 | 1.72 | 1.06 | 1.54 | 0.93 | 1.88 | 3.23 | 1 |
| 491 | 39442 | R51817 | 0.84 | 0.37 | 1.72 | 0.66 | 0.78 | 1.74 | 2.38 | 3.05 | 1 |
| 492 | 285888 | N25344 | 2.24 | 1.30 | 1.72 | 1.67 | 0.93 | 0.90 | 1.07 | 4.02 | 1 |
| 493 | 277185 | N40951 | 3.91 | 2.28 | 1.72 | 1.84 | 0.82 | 3.82 | 1.28 | 0.84 | 1 |
| 494 | 135085 | R31413 | 5.42 | 3.18 | 1.71 | 4.03 | 2.84 | 0.90 | 0.28 | 0.53 | 1 |
| 495 | 502527 | AA158873 | 0.07 | 0.04 | 1.71 | 0.71 | 5.02 | 1.23 | 0.64 | 0.97 | 1 |
| 496 | 771323 | AA476240 | 3.23 | 1.89 | 1.71 | 1.27 | 1.12 | 1.19 | 3.56 | 1.43 | 1 |
| 497 | 322788 | W39609 | 0.73 | 0.42 | 1.71 | 3.09 | 2.35 | 1.35 | 0.77 | 0.98 | 1 |
| 498 | 809503 | AA454582 | 1.03 | 0.60 | 1.71 | 0.84 | 1.08 | 4.28 | 1.40 | 0.97 | 1 |
| 499 | 713177 | AA285053 | 1.43 | 0.84 | 1.71 | 1.27 | 1.80 | 1.15 | 1.01 | 3.30 | 1 |
| 500 | 71312 | T47625 | 2.44 | 1.43 | 1.71 | 3.25 | 1.32 | 1.48 | 1.81 | 0.89 | 1 |
| 501 | 51950 | H24316 | 4.32 | 2.53 | 1.71 | 1.19 | 1.46 | 1.35 | 3.23 | 1.30 | 1 |
| 502 | 382720 | AA018215 | 0.78 | 0.48 | 1.71 | 1.46 | 5.09 | 0.50 | 0.38 | 1.10 | 1 |
| 503 | 123081 | R00128 | 3.22 | 1.89 | 1.70 | 3.97 | 1.52 | 1.28 | 0.71 | 1.08 | 1 |
| 504 | 126829 | R07184 | 3.88 | 2.28 | 1.70 | 1.20 | 1.17 | 3.25 | 1.55 | 1.34 | 1 |
| 505 | 431482 | AA708870 | 0.72 | 0.42 | 1.70 | 1.78 | 1.38 | 3.79 | 0.91 | 0.68 | 1 |
| 506 | 825742 | AA504845 | 1.43 | 0.84 | 1.70 | 4.28 | 0.88 | 0.65 | 1.40 | 1.31 | 1 |
| 507 | 731290 | AA418827 | 32.92 | 19.38 | 1.70 | 3.03 | 0.58 | 0.58 | 2.33 | 1.97 | 1 |
| 508 | 855238 | AA782292 | 8.41 | 4.95 | 1.70 | 1.82 | 0.91 | 3.05 | 1.34 | 1.37 | 1 |
| 509 | 488591 | AA042990 | 5.61 | 3.30 | 1.70 | 3.67 | 1.23 | 0.41 | 0.91 | 2.27 | 1 |
| 510 | 1031903 | AA809731 | 3.21 | 1.89 | 1.70 | 4.10 | 1.36 | 1.27 | 1.00 | 0.75 | 1 |
| 511 | 743353 | AA400317 | 2.82 | 1.66 | 1.70 | 3.79 | 0.71 | 1.43 | 1.36 | 1.20 | 1 |
| 512 | 824723 | AA488986 | 27.00 | 15.91 | 1.70 | 2.63 | 1.20 | 0.21 | 1.25 | 3.20 | 1 |
| 513 | 123928 | R01515 | 2.38 | 1.40 | 1.70 | 3.32 | 1.12 | 0.48 | 1.09 | 2.48 | 1 |
| 514 | 430720 | AA878087 | 1.00 | 0.59 | 1.70 | 1.46 | 1.23 | 3.19 | 1.03 | 1.57 | 1 |
| 515 | 742115 | AA405800 | 2.72 | 1.81 | 1.69 | 1.41 | 1.18 | 3.01 | 1.58 | 1.30 | 1 |
| 516 | 128474 | R08716 | 1.73 | 1.02 | 1.69 | 0.67 | 0.85 | 1.05 | 3.34 | 2.53 | 1 |
| 517 | 755274 | AA498348 | 2.77 | 1.84 | 1.69 | 1.42 | 1.77 | 3.08 | 1.70 | 0.49 | 1 |
| 518 | 345849 | W70343 | 0.83 | 0.37 | 1.89 | 0.68 | 1.24 | 3.78 | 1.43 | 1.32 | 1 |
| 519 | 31388 | R42738 | 0.50 | 0.30 | 1.69 | 0.70 | 0.85 | 4.90 | 0.78 | 1.19 | 1 |
| 520 | 825822 | AA505141 | 49.12 | 29.18 | 1.68 | 1.13 | 1.34 | 0.70 | 1.57 | 3.68 | 1 |
| 521 | 133179 | R25403 | 1.99 | 1.18 | 1.88 | 0.80 | 0.74 | 2.03 | 3.55 | 1.29 | 1 |
| 522 | 23819 | R39448 | 4.78 | 2.85 | 1.88 | 0.84 | 0.82 | 4.94 | 0.99 | 0.80 | 1 |
| 523 | 448417 | AA777555 | 4.20 | 2.50 | 1.68 | 1.33 | 0.71 | 0.44 | 2.23 | 3.88 | 1 |
| 524 | 828338 | AA198488 | 5.05 | 3.02 | 1.67 | 0.72 | 0.69 | 5.10 | 0.98 | 0.91 | 1 |
| 525 | 162310 | H28091 | 7.21 | 4.30 | 1.87 | 3.26 | 0.83 | 0.44 | 1.37 | 2.46 | 1 |
| 526 | 824271 | AA181179 | 30.10 | 17.98 | 1.87 | 3.11 | 0.80 | 1.45 | 1.80 | 1.41 | 1 |
| 527 | 40027 | R53943 | 1.37 | 0.82 | 1.67 | 1.11 | 1.79 | 3.38 | 1.26 | 0.82 | 1 |
| 528 | 415851 | W88282 | 2.11 | 1.28 | 1.67 | 0.76 | 1.71 | 0.44 | 1.72 | 3.73 | 1 |
| 529 | 383081 | AA019335 | 11.00 | 8.59 | 1.67 | 0.95 | 0.82 | 3.41 | 1.18 | 2.00 | 1 |
| 530 | 327799 | W23831 | 0.73 | 0.44 | 1.87 | 0.71 | 0.73 | 1.07 | 5.42 | 0.42 | 1 |
| 531 | 548800 | AA084517 | 1.46 | 0.88 | 1.87 | 1.48 | 1.18 | 0.93 | 1.32 | 3.42 | 1 |
| 532 | 731311 | AA416787 | 3.48 | 2.08 | 1.68 | 3.15 | 0.43 | 0.58 | 1.91 | 2.25 | 1 |
| 533 | 76169 | T59858 | 21.82 | 13.12 | 1.66 | 1.78 | 0.70 | 0.24 | 1.30 | 4.29 | 1 |
| 534 | 145491 | R77512 | 3.50 | 2.10 | 1.66 | 0.60 | 1.02 | 2.04 | 3.51 | 1.15 | 1 |
| 535 | 814320 | AA459110 | 0.99 | 0.60 | 1.66 | 3.33 | 2.97 | 0.50 | 0.76 | 0.75 | 1 |
| 536 | 727028 | AA402760 | 0.67 | 0.40 | 1.86 | 1.15 | 3.47 | 1.48 | 0.96 | 1.25 | 1 |
| 537 | 23443 | R38878 | 0.98 | 0.59 | 1.66 | 0.67 | 1.53 | 4.58 | 0.68 | 0.86 | 1 |
| 538 | 41648 | R52796 | 0.97 | 0.58 | 1.66 | 0.67 | 1.35 | 0.70 | 1.42 | 4.16 | 1 |
| 539 | 1472479 | AA872257 | 44.42 | 26.77 | 1.66 | 1.12 | 2.02 | 0.87 | 0.77 | 3.52 | 1 |
| 540 | 245824 | N77263 | 2.31 | 1.39 | 1.66 | 4.29 | 0.94 | 0.70 | 0.71 | 1.65 | 1 |
| 541 | 490647 | AA115742 | 3.55 | 2.14 | 1.66 | 1.30 | 0.67 | 0.79 | 2.35 | 3.17 | 1 |
| 542 | 789798 | AA429034 | 2.35 | 1.42 | 1.66 | 1.85 | 0.84 | 0.89 | 3.61 | 1.08 | 1 |
| 543 | 841308 | AA487215 | 33.63 | 20.37 | 1.65 | 1.20 | 1.07 | 0.32 | 2.14 | 3.52 | 1 |
| 544 | 88061 | T52840 | 1.21 | 0.73 | 1.65 | 0.57 | 3.03 | 2.59 | 0.93 | 1.13 | 1 |
| 545 | 45391 | H08194 | 1.25 | 0.76 | 1.65 | 0.46 | 0.58 | 6.16 | 0.49 | 0.56 | 1 |
| 546 | 810901 | AA459293 | 2.47 | 1.50 | 1.65 | 3.14 | 1.44 | 1.40 | 1.33 | 0.92 | 1 |
| 547 | 427998 | AA001841 | 3.41 | 2.07 | 1.65 | 0.36 | 5.71 | 1.20 | 0.46 | 0.51 | 1 |
| 548 | 50928 | H19327 | 8.31 | 5.04 | 1.65 | 1.00 | 1.05 | 0.94 | 1.66 | 3.60 | 1 |
| 549 | 1035765 | AA629117 | 14.84 | 9.01 | 1.65 | 0.31 | 2.94 | 0.81 | 0.20 | 3.98 | 1 |
| 550 | 447404 | AA702327 | 3.35 | 2.03 | 1.65 | 0.91 | 3.13 | 1.35 | 0.77 | 2.08 | 1 |
| 551 | 825060 | AA489219 | 0.15 | 0.09 | 1.64 | 4.25 | 1.26 | 0.73 | 0.65 | 1.33 | 1 |
| 552 | 137275 | R38587 | 1.57 | 0.95 | 1.64 | 3.97 | 0.70 | 1.10 | 1.62 | 0.83 | 1 |
| 553 | 293539 | N63727 | 1.06 | 0.65 | 1.64 | 3.03 | 1.53 | 1.15 | 1.00 | 1.51 | 1 |
| 554 | 845380 | AA644099 | 2.39 | 1.46 | 1.64 | 3.08 | 2.33 | 0.43 | 0.87 | 1.51 | 1 |
| 555 | 84580 | T74023 | 3.38 | 2.06 | 1.64 | 3.59 | 0.95 | 1.76 | 1.19 | 0.71 | 1 |
| 556 | 854833 | AA630445 | 5.81 | 3.54 | 1.64 | 1.23 | 0.80 | 3.24 | 2.04 | 0.89 | 1 |
| 557 | 1070062 | AA599741 | 0.30 | 0.18 | 1.64 | 3.21 | 1.32 | 1.36 | 1.00 | 1.31 | 1 |
| 558 | 40138 | R53951 | 6.88 | 4.20 | 1.64 | 1.35 | 3.63 | 1.30 | 0.97 | 0.95 | 1 |
| 559 | 810358 | AA464163 | 5.37 | 3.28 | 1.64 | 1.10 | 0.96 | 2.07 | 3.30 | 0.74 | 1 |
| 560 | 824720 | AA488976 | 2.18 | 1.33 | 1.63 | 2.06 | 1.17 | 3.27 | 0.96 | 0.70 | 1 |
| 561 | 244391 | N52837 | 0.78 | 0.48 | 1.63 | 1.12 | 2.11 | 3.03 | 1.01 | 0.89 | 1 |
| 562 | 814547 | AA480870 | 0.19 | 0.12 | 1.63 | 1.68 | 1.76 | 3.29 | 0.77 | 0.65 | 1 |
| 563 | 840990 | AA488889 | 5.20 | 3.20 | 1.83 | 0.62 | 1.86 | 0.51 | 1.47 | 3.69 | 1 |
| 564 | 383055 | AA019320 | 0.57 | 0.35 | 1.63 | 0.83 | 0.86 | 4.65 | 1.18 | 0.61 | 1 |
| 565 | 288959 | N59808 | 1.06 | 0.65 | 1.63 | 1.20 | 1.25 | 3.35 | 1.02 | 1.32 | 1 |
| 566 | 1048789 | AA821332 | 1.66 | 1.02 | 1.63 | 0.67 | 3.33 | 2.52 | 0.79 | 0.82 | 1 |
| 567 | 1240220 | AA788970 | 6.73 | 4.14 | 1.62 | 4.08 | 0.48 | 0.87 | 1.25 | 1.44 | 1 |
| 568 | 383528 | AA878975 | 1.85 | 1.14 | 1.62 | 0.93 | 1.17 | 3.94 | 1.10 | 0.99 | 1 |
| 569 | 280950 | N50843 | 2.57 | 1.59 | 1.62 | 1.19 | 1.00 | 3.43 | 1.20 | 1.29 | 1 |
| 570 | 825889 | AA505082 | 7.82 | 4.82 | 1.62 | 1.18 | 0.86 | 3.58 | 1.40 | 1.08 | 1 |
| 571 | 784216 | AA448866 | 0.09 | 0.05 | 1.62 | 2.53 | 0.97 | 3.07 | 0.97 | 0.58 | 1 |
| 572 | 435992 | AA703219 | 0.64 | 0.40 | 1.62 | 1.92 | 1.00 | 3.47 | 0.68 | 1.02 | 1 |
| 573 | 1636251 | A1017240 | 0.13 | 0.08 | 1.61 | 1.60 | 1.81 | 1.30 | 0.46 | 3.07 | 1 |
| 574 | 782448 | AA431571 | 5.45 | 3.39 | 1.61 | 3.30 | 1.43 | 0.44 | 1.11 | 1.76 | 1 |
| 575 | 325526 | W52355 | 3.93 | 2.44 | 1.61 | 3.38 | 1.24 | 0.85 | 1.29 | 1.27 | 1 |
| 576 | 325111 | W47000 | 1.14 | 0.71 | 1.81 | 1.41 | 0.95 | 3.46 | 1.24 | 0.98 | 1 |
| 577 | 109316 | T80924 | 8.43 | 4.00 | 1.61 | 0.54 | 6.24 | 0.43 | 0.48 | 0.34 | 1 |
| 578 | 49842 | H29268 | 0.62 | 0.39 | 1.50 | 3.89 | 1.01 | 1.05 | 1.23 | 0.84 | 1 |
| 579 | 122684 | T99011 | 0.88 | 0.53 | 1.60 | 3.39 | 1.35 | 1.10 | 1.08 | 1.11 | 1 |
| 580 | 826173 | AA521431 | 7.28 | 4.55 | 1.60 | 1.05 | 0.43 | 2.21 | 3.08 | 1.24 | 1 |
| 581 | 137940 | R63106 | 3.44 | 2.15 | 1.80 | 1.54 | 1.28 | 0.59 | 0.87 | 3.71 | 1 |
| 582 | 137254 | R37519 | 1.20 | 0.75 | 1.60 | 2.11 | 3.17 | 1.05 | 0.89 | 0.78 | 1 |
| 583 | 753982 | AA479967 | 2.78 | 1.74 | 1.60 | 4.05 | 0.62 | 0.53 | 1.47 | 1.32 | 1 |
| 584 | 265103 | N21338 | 1.74 | 1.09 | 1.60 | 0.64 | 1.15 | 3.86 | 1.46 | 0.88 | 1 |
| 585 | 381974 | AA001449 | 11.24 | 7.03 | 1.60 | 1.10 | 0.47 | 0.19 | 4.51 | 1.71 | 1 |
| 586 | 267541 | N24538 | 0.82 | 0.51 | 1.60 | 1.29 | 1.08 | 3.05 | 1.30 | 1.26 | 1 |
| 587 | 753378 | AA411686 | 9.99 | 8.26 | 1.60 | 1.22 | 0.78 | 1.31 | 1.45 | 3.23 | 1 |
| 588 | 73838 | T55728 | 7.12 | 4.46 | 1.60 | 1.04 | 0.73 | 0.93 | 2.13 | 3.15 | 1 |
| 589 | 824490 | AA187207 | 2.88 | 1.81 | 1.59 | 1.44 | 3.29 | 0.78 | 1.11 | 1.35 | 1 |
| 590 | 377731 | AA058232 | 5.53 | 3.47 | 1.59 | 1.12 | 1.04 | 0.30 | 2.23 | 3.27 | 1 |
| 591 | 837891 | AA434092 | 7.51 | 4.73 | 1.59 | 1.30 | 0.67 | 3.47 | 1.70 | 0.79 | 1 |
| 592 | 480403 | AA677534 | 2.13 | 1.34 | 1.59 | 0.90 | 2.31 | 3.54 | 0.74 | 0.43 | 1 |
| 593 | 384083 | AA021628 | 0.45 | 0.29 | 1.58 | 1.02 | 1.46 | 3.18 | 0.90 | 1.38 | 1 |
| 594 | 41589 | R52901 | 1.58 | 0.99 | 1.58 | 0.52 | 2.82 | 3.80 | 0.34 | 0.43 | 1 |
| 595 | 131791 | R24508 | 2.77 | 1.75 | 1.58 | 1.16 | 1.05 | 3.47 | 1.44 | 0.78 | 1 |
| 596 | 133273 | R26960 | 2.17 | 1.37 | 1.58 | 3.17 | 0.49 | 0.44 | 1.28 | 2.52 | 1 |
| 597 | 229385 | H74265 | 0.68 | 0.43 | 1.58 | 3.56 | 1.35 | 0.65 | 1.04 | 1.30 | 1 |
| 598 | 784085 | AA443832 | 1.14 | 0.72 | 1.58 | 0.82 | 1.75 | 3.68 | 1.00 | 0.63 | 1 |
| 599 | 852548 | AA663123 | 4.72 | 3.00 | 1.57 | 3.51 | 1.08 | 1.10 | 1.19 | 0.99 | 1 |
| 600 | 24855 | R38952 | 0.54 | 0.34 | 1.57 | 3.97 | 1.28 | 1.00 | 0.76 | 0.86 | 1 |
| 601 | 196387 | R91503 | 0.48 | 0.30 | 1.57 | 3.42 | 1.24 | 0.97 | 1.01 | 1.23 | 1 |
| 602 | 825418 | AA504285 | 13.96 | 8.68 | 1.57 | 1.43 | 0.78 | 0.22 | 1.36 | 4.07 | 1 |
| 603 | 725745 | AA399223 | 1.75 | 1.11 | 1.57 | 1.05 | 1.09 | 1.68 | 3.26 | 0.78 | 1 |
| 604 | 143450 | R74478 | 2.91 | 1.85 | 1.57 | 0.38 | 1.13 | 4.39 | 1.17 | 0.79 | 1 |
| 605 | 247680 | N58170 | 0.84 | 0.54 | 1.57 | 3.17 | 1.63 | 1.05 | 0.97 | 1.04 | 1 |
| 606 | 811880 | AA454634 | 9.80 | 6.24 | 1.57 | 0.59 | 1.72 | 3.42 | 0.95 | 1.16 | 1 |
| 607 | 582395 | AA214031 | 0.74 | 0.47 | 1.57 | 1.36 | 0.75 | 3.21 | 1.07 | 1.45 | 1 |
| 608 | 341805 | W60845 | 2.30 | 1.47 | 1.57 | 3.13 | 0.60 | 1.03 | 2.24 | 0.84 | 1 |
| 609 | 454771 | AA677287 | 4.72 | 3.03 | 1.56 | 0.87 | 4.88 | 0.95 | 0.75 | 0.36 | 1 |
| 610 | 462858 | AA705319 | 0.47 | 0.30 | 1.56 | 3.66 | 0.85 | 0.83 | 0.99 | 1.68 | 1 |
| 611 | 280459 | N47240 | 24.12 | 15.46 | 1.56 | 0.60 | 0.79 | 3.91 | 1.26 | 1.23 | 1 |
| 612 | 711918 | AA282134 | 0.85 | 0.55 | 1.56 | 3.78 | 1.50 | 0.77 | 0.72 | 1.03 | 1 |
| 613 | 825854 | AA505051 | 1.10 | 0.71 | 1.56 | 3.72 | 1.28 | 0.88 | 0.97 | 0.97 | 1 |
| 614 | 1049009 | AA778653 | 1.24 | 0.80 | 1.56 | 1.92 | 0.98 | 1.06 | 3.20 | 0.63 | 1 |
| 615 | 52079 | H23202 | 4.29 | 2.75 | 1.56 | 1.13 | 1.04 | 0.33 | 1.98 | 3.31 | 1 |
| 616 | 25384 | R12808 | 1.08 | 0.69 | 1.56 | 1.18 | 1.30 | 3.22 | 0.99 | 1.09 | 1 |
| 617 | 471892 | AA035171 | 6.47 | 4.16 | 1.55 | 1.06 | 0.67 | 0.50 | 2.24 | 3.29 | 1 |
| 618 | 24131 | R38017 | 0.43 | 0.27 | 1.55 | 1.05 | 1.12 | 3.55 | 1.01 | 1.04 | 1 |
| 619 | 1049230 | AA620715 | 4.19 | 2.70 | 1.55 | 0.12 | 1.08 | 1.95 | 3.79 | 0.82 | 1 |
| 620 | 34889 | R44447 | 9.34 | 6.02 | 1.55 | 3.99 | 0.84 | 1.20 | 1.11 | 0.60 | 1 |
| 621 | 431505 | AA676225 | 5.83 | 3.76 | 1.55 | 3.52 | 0.50 | 0.76 | 0.84 | 2.13 | 1 |
| 622 | 250988 | H98557 | 0.37 | 0.24 | 1.55 | 0.73 | 0.87 | 1.27 | 1.60 | 3.26 | 1 |
| 623 | 897926 | AA598809 | 1.23 | 0.80 | 1.55 | 1.12 | 1.21 | 3.23 | 1.20 | 0.97 | 1 |
| 624 | 165828 | R86847 | 1.60 | 1.04 | 1.54 | 0.83 | 0.95 | 3.32 | 1.51 | 1.10 | 1 |
| 625 | 824920 | AA489018 | 6.49 | 4.21 | 1.54 | 0.89 | 0.89 | 4.08 | 0.95 | 0.93 | 1 |
| 626 | 502874 | AA135886 | 0.75 | 0.49 | 1.54 | 3.14 | 1.27 | 0.81 | 1.27 | 1.21 | 1 |
| 627 | 49203 | H15695 | 0.36 | 0.23 | 1.54 | 3.07 | 0.90 | 1.13 | 1.11 | 1.49 | 1 |
| 628 | 486208 | AA040617 | 3.32 | 2.16 | 1.54 | 1.12 | 1.11 | 0.43 | 1.73 | 3.31 | 1 |
| 629 | 1505892 | AA879437 | 3.30 | 2.14 | 1.54 | 3.80 | 0.53 | 0.68 | 1.47 | 1.20 | 1 |
| 630 | 39593 | R51912 | 0.29 | 0.19 | 1.54 | 3.16 | 1.24 | 1.10 | 1.24 | 0.93 | 1 |
| 631 | 283142 | N51335 | 8.63 | 5.82 | 1.54 | 1.10 | 1.01 | 3.39 | 1.16 | 1.01 | 1 |
| 632 | 767284 | AA418414 | 1.07 | 0.89 | 1.53 | 0.89 | 1.88 | 3.19 | 1.06 | 0.84 | 1 |
| 633 | 284247 | N52178 | 116.27 | 75.80 | 1.53 | 0.58 | 0.72 | 3.62 | 1.47 | 1.28 | 1 |
| 634 | 298862 | N75358 | 0.85 | 0.55 | 1.53 | 1.21 | 1.30 | 3.01 | 1.37 | 0.76 | 1 |
| 635 | 213917 | H72959 | 1.09 | 0.71 | 1.53 | 3.35 | 1.10 | 0.88 | 1.38 | 0.94 | 1 |
| 636 | 380478 | AA015793 | 0.51 | 0.33 | 1.53 | 3.19 | 1.75 | 0.86 | 0.87 | 0.98 | 1 |
| 637 | 268300 | N26724 | 1.70 | 1.11 | 1.53 | 1.53 | 0.85 | 3.27 | 1.06 | 0.93 | 1 |
| 638 | 505225 | AA142922 | 15.46 | 10.13 | 1.53 | 0.93 | 1.22 | 0.45 | 1.18 | 3.85 | 1 |
| 639 | 149742 | R82733 | 0.85 | 0.56 | 1.52 | 3.16 | 1.37 | 1.12 | 1.30 | 0.67 | 1 |
| 640 | 628295 | AA196485 | 1.15 | 0.76 | 1.52 | 1.26 | 1.39 | 3.18 | 0.86 | 0.91 | 1 |
| 641 | 1472743 | AA872397 | 2.35 | 154 | 1.52 | 4.69 | 0.70 | 0.42 | 0.86 | 0.95 | 1 |
| 642 | 129024 | R10378 | 3.59 | 2.38 | 1.52 | 1.44 | 4.17 | 1.25 | 0.42 | 0.33 | 1 |
| 643 | 135800 | R33103 | 0.94 | 0.62 | 1.52 | 3.42 | 1.03 | 1.06 | 1.33 | 0.77 | 1 |
| 644 | 435470 | AA701502 | 13.85 | 9.11 | 1.52 | 0.54 | 3.56 | 1.37 | 0.93 | 1.20 | 1 |
| 645 | 564803 | AA129552 | 1.34 | 0.88 | 1.52 | 3.72 | 1.19 | 1.03 | 0.77 | 0.89 | 1 |
| 646 | 205497 | H57857 | 3.23 | 2.12 | 1.52 | 3.20 | 0.74 | 1.43 | 1.29 | 0.93 | 1 |
| 647 | 151201 | H02307 | 3.84 | 2.53 | 1.52 | 3.15 | 1.25 | 0.39 | 1.03 | 1.77 | 1 |
| 648 | 713922 | AA290737 | 6.20 | 4.09 | 1.52 | 0.67 | 1.59 | 0.38 | 1.38 | 3.55 | 1 |
| 649 | 279481 | N48809 | 37.32 | 24.63 | 1.52 | 0.63 | 0.80 | 3.88 | 1.17 | 1.10 | 1 |
| 650 | 53393 | R16245 | 0.76 | 0.50 | 1.52 | 3.22 | 1.77 | 1.27 | 0.56 | 0.76 | 1 |
| 651 | 592594 | AA159578 | 12.33 | 8.15 | 1.51 | 0.52 | 1.42 | 3.16 | 1.34 | 1.12 | 1 |
| 652 | 81408 | T60149 | 1.81 | 1.20 | 1.51 | 3.37 | 1.30 | 0.75 | 0.68 | 1.47 | 1 |
| 653 | 399421 | AA733177 | 4.13 | 2.73 | 1.51 | 0.59 | 1.21 | 1.96 | 0.51 | 3.30 | 1 |
| 654 | 324751 | AA284113 | 0.28 | 0.18 | 1.51 | 4.33 | 0.82 | 0.67 | 0.68 | 1.07 | 1 |
| 655 | 27769 | R40176 | 0.71 | 0.47 | 1.51 | 4.05 | 0.93 | 0.88 | 0.57 | 1.13 | 1 |
| 656 | 84613 | T74105 | 52.58 | 34.81 | 1.51 | 1.64 | 0.95 | 0.26 | 1.24 | 3.46 | 1 |
| 657 | 787176 | AA424582 | 3.40 | 2.25 | 1.51 | 0.44 | 1.82 | 3.44 | 0.95 | 0.90 | 1 |
| 658 | 307231 | N93428 | 1.17 | 0.78 | 1.51 | 3.61 | 0.80 | 0.68 | 0.70 | 1.75 | 1 |
| 659 | 279249 | N48580 | 0.53 | 0.35 | 1.51 | 0.48 | 0.58 | 0.71 | 5.02 | 0.74 | 1 |
| 660 | 878277 | AA775810 | 2.65 | 1.76 | 1.50 | 4.08 | 0.71 | 0.58 | 0.94 | 1.21 | 1 |
| 661 | 703808 | AA278320 | 2.88 | 1.92 | 1.50 | 0.82 | 1.39 | 3.07 | 0.96 | 1.27 | 1 |
| 662 | 80772 | T63031 | 2.20 | 1.48 | 1.50 | 1.10 | 0.79 | 3.20 | 1.61 | 0.79 | 1 |
| 663 | 451351 | AA707008 | 1.12 | 0.75 | 1.50 | 0.99 | 0.94 | 3.06 | 1.52 | 0.98 | 1 |
| 664 | 325088 | W48985 | 4.80 | 3.21 | 1.50 | 0.69 | 0.69 | 3.96 | 1.49 | 0.66 | 1 |
| 665 | 731051 | AA421284 | 0.71 | 0.47 | 1.50 | 0.82 | 0.99 | 3.58 | 1.32 | 0.78 | 1 |
| 666 | 320797 | W31725 | 1.79 | 1.20 | 1.50 | 3.40 | 0.81 | 0.81 | 1.20 | 1.26 | 1 |
| 667 | 34442 | R44985 | 1.08 | 0.71 | 1.50 | 3.47 | 1.17 | 0.76 | 0.96 | 1.11 | 1 |
| 668 | 320392 | W16832 | 4.59 | 3.07 | 1.49 | 0.77 | 1.01 | 0.50 | 0.76 | 4.43 | 1 |
| 669 | 897958 | AA598817 | 1.17 | 0.78 | 1.49 | 0.39 | 8.15 | 0.29 | 0.27 | 0.37 | 1 |
| 670 | 272529 | 1435888 | 1.51 | 1.01 | 1.49 | 0.44 | 1.05 | 3.40 | 1.75 | 0.82 | 1 |
| 671 | 417081 | W87826 | 2.45 | 1.64 | 1.49 | 0.55 | 1.25 | 3.49 | 1.35 | 0.83 | 1 |
| 672 | 435577 | AA703161 | 16.03 | 10.74 | 1.49 | 0.37 | 0.58 | 3.49 | 1.72 | 1.30 | 1 |
| 673 | 814739 | AA454928 | 0.65 | 0.44 | 1.49 | 1.21 | 1.34 | 3.53 | 0.85 | 0.52 | 1 |
| 674 | 127120 | R08121 | 1.73 | 1.16 | 1.49 | 0.63 | 1.31 | 1.15 | 3.20 | 1.17 | 1 |
| 675 | 1472152 | AA873578 | 2.15 | 1.44 | 1.49 | 0.87 | 3.64 | 0.42 | 0.46 | 2.08 | 1 |
| 676 | 503617 | AA131406 | 1.23 | 0.82 | 1.49 | 3.20 | 1.45 | 0.52 | 0.63 | 1.65 | 1 |
| 677 | 132636 | R26785 | 10.16 | 6.83 | 1.49 | 0.20 | 0.95 | 3.00 | 2.09 | 1.19 | 1 |
| 678 | 270750 | N33443 | 0.78 | 0.53 | 1.49 | 3.51 | 1.01 | 0.95 | 1.07 | 0.90 | 1 |
| 679 | 34007 | R44544 | 0.60 | 0.41 | 1.49 | 0.57 | 1.45 | 3.09 | 1.56 | 0.76 | 1 |
| 680 | 753162 | AA400457 | 6.76 | 4.56 | 1.48 | 3.68 | 0.97 | 0.98 | 0.93 | 0.86 | 1 |
| 681 | 743481 | AAB09392 | 1.09 | 0.73 | 1.48 | 3.07 | 0.93 | 0.91 | 1.23 | 1.27 | 1 |
| 682 | 384224 | AA702077 | 22.54 | 15.21 | 1.48 | 0.56 | 0.73 | 3.04 | 1.76 | 1.32 | 1 |
| 683 | 128165 | R12385 | 1.46 | 0.98 | 1.48 | 1.13 | 0.92 | 3.34 | 1.00 | 1.01 | 1 |
| 684 | 150897 | H03438 | 0.50 | 0.34 | 1.46 | 1.51 | 1.06 | 0.97 | 0.68 | 3.17 | 1 |
| 685 | 294503 | N69528 | 8.90 | 8.02 | 1.48 | 1.01 | 0.91 | 3.63 | 0.91 | 0.93 | 1 |
| 686 | 823863 | AA489729 | 0.74 | 0.50 | 1.48 | 0.77 | 0.78 | 1.59 | 3.23 | 1.01 | 1 |
| 687 | 361291 | AA017468 | 1.99 | 1.35 | 1.48 | 0.85 | 3.02 | 2.15 | 0.65 | 0.72 | 1 |
| 688 | 1600281 | AA962119 | 1.84 | 1.25 | 1.47 | 0.90 | 1.05 | 3.16 | 1.32 | 0.94 | 1 |
| 689 | 435536 | AA701900 | 1.18 | 0.80 | 1.47 | 1.06 | 1.02 | 3.27 | 0.97 | 1.04 | 1 |
| 690 | 248117 | N58417 | 0.68 | 0.46 | 1.47 | 3.96 | 1.49 | 0.35 | 0.74 | 0.81 | 1 |
| 691 | 451546 | AA707400 | 2.00 | 1.40 | 1.47 | 0.94 | 0.97 | 3.63 | 1.28 | 0.53 | 1 |
| 692 | 44975 | H08899 | 9.58 | 6.52 | 1.47 | 1.47 | 0.84 | 0.80 | 0.98 | 3.25 | 1 |
| 693 | 257504 | N30285 | 7.47 | 5.09 | 1.47 | 1.24 | 1.15 | 0.74 | 0.95 | 3.26 | 1 |
| 694 | 845477 | AA644211 | 0.67 | 0.46 | 1.47 | 1.14 | 0.79 | 3.88 | 0.81 | 0.71 | 1 |
| 695 | 430864 | AA878203 | 1.15 | 0.79 | 1.46 | 0.90 | 0.81 | 4.22 | 0.78 | 0.61 | 1 |
| 696 | 700571 | AA283949 | 0.86 | 0.59 | 1.46 | 3.49 | 1.12 | 0.64 | 0.73 | 1.34 | 1 |
| 697 | 415868 | W88214 | 0.72 | 0.49 | 1.46 | 3.61 | 0.88 | 0.87 | 0.96 | 0.98 | 1 |
| 698 | 133150 | R26444 | 1.03 | 0.70 | 1.46 | 3.33 | 1.45 | 0.72 | 1.01 | 0.80 | 1 |
| 699 | 50918 | H19320 | 0.57 | 0.39 | 1.46 | 3.07 | 1.19 | 1.04 | 0.86 | 1.15 | 1 |
| 700 | 772425 | AA405569 | 0.92 | 0.83 | 1.46 | 3.02 | 1.05 | 0.88 | 1.17 | 1.16 | 1 |
| 701 | 789069 | AA453085 | 0.22 | 0.15 | 1.46 | 3.96 | 1.33 | 0.51 | 0.67 | 0.80 | 1 |
| 702 | 219888 | H84857 | 34.94 | 24.04 | 1.45 | 0.62 | 0.65. | 3.91 | 0.99 | 1.09 | 1 |
| 703 | 109863 | T88721 | 8.23 | 5.67 | 1.45 | 0.69 | 1.34 | 0.83 | 1.00 | 3.41 | 1 |
| 704 | 39977 | R52526 | 0.57 | 0.39 | 1.45 | 0.74 | 1.35 | 3.33 | 0.98 | 0.87 | 1 |
| 705 | 397432 | AA701046 | 6.82 | 4.70 | 1.45 | 1.12 | 0.86 | 3.23 | 1.32 | 0.72 | 1 |
| 706 | 208377 | H62839 | 134.60 | 92.77 | 1.45 | 0.58 | 0.82 | 3.73 | 1.08 | 1.06 | 1 |
| 707 | 883278 | AA213889 | 0.80 | 0.55 | 1.45 | 1.39 | 1.28 | 3.01 | 0.65 | 0.92 | 1 |
| 708 | 197731 | R93591 | 3.30 | 2.27 | 1.45 | 0.65 | 0.67 | 3.93 | 1.06 | 0.95 | 1 |
| 709 | 140174 | R66082 | 1.52 | 1.05 | 1.45 | 0.69 | 0.89 | 3.14 | 2.09 | 0.64 | 1 |
| 710 | 886492 | AA233070 | 1.90 | 1.31 | 1.45 | 3.01 | 1.08 | 0.89 | 1.37 | 0.92 | 1 |
| 711 | 840686 | AA488064 | 10.78 | 7.46 | 1.45 | 1.06 | 0.84 | 3.04 | 1.72 | 0.77 | 1 |
| 712 | 133053 | R26206 | 1.51 | 1.05 | 1.45 | 0.94 | 1.08 | 3.87 | 0.71 | 0.63 | 1 |
| 713 | 291426 | N67766 | 4.93 | 3.41 | 1.44 | 0.47 | 1.10 | 3.63 | 1.61 | 0.41 | 1 |
| 714 | 1472735 | AA872383 | 104.94 | 72.70 | 1.44 | 0.15 | 0.49 | 0.42 | 2.98 | 3.19 | 1 |
| 715 | 814018 | AA455859 | 1.56 | 1.08 | 1.44 | 1.55 | 0.87 | 3.03 | 0.91 | 0.85 | 1 |
| 716 | 971387 | AA683050 | 225.26 | 156.38 | 1.44 | 1.18 | 1.34 | 3.09 | 0.81 | 0.78 | 1 |
| 717 | 153411 | R47979 | 62.25 | 43.22 | 1.44 | 3.00 | 0.76 | 0.13 | 0.68 | 2.83 | 1 |
| 718 | 290162 | N63286 | 0.69 | 0.48 | 1.44 | 3.26 | 0.93 | 0.72 | 1.07 | 1.22 | 1 |
| 719 | 418081 | W90067 | 0.32 | 0.22 | 1.44 | 0.86 | 1.29 | 0.88 | 1.00 | 3.17 | 1 |
| 720 | 121530 | T97890 | 1.84 | 1.28 | 1.44 | 3.63 | 1.00 | 0.64 | 0.86 | 1.06 | 1 |
| 721 | 143759 | R78505 | 2.68 | 1.87 | 1.43 | 0.60 | 1.25 | 3.27 | 1.16 | 0.87 | 1 |
| 722 | 489800 | AA099820 | 0.37 | 0.26 | 1.43 | 3.19 | 0.61 | 1.05 | 1.33 | 0.98 | 1 |
| 723 | 112482 | T90980 | 10.24 | 7.16 | 1.43 | 1.37 | 1.38 | 0.93 | 0.42 | 3.05 | 1 |
| 724 | 1555523 | AA974971 | 1.47 | 1.03 | 1.43 | 0.65 | 0.90 | 0.70 | 0.89 | 4.02 | 1 |
| 725 | 24915 | R39066 | 0.64 | 0.44 | 1.43 | 0.63 | 0.80 | 4.46 | 0.81 | 0.46 | 1 |
| 726 | 757451 | AA437241 | 1.18 | 0.83 | 1.43 | 3.19 | 0.90 | 0.77 | 0.62 | 1.84 | 1 |
| 727 | 239448 | H70047 | 0.71 | 0.49 | 1.43 | 1.14 | 1.20 | 0.59 | 1.17 | 3.03 | 1 |
| 728 | 392607 | AA708240 | 41.56 | 29.18 | 1.42 | 0.51 | 0.67 | 3.69 | 1.03 | 1.22 | 1 |
| 729 | 744944 | AA825890 | 6.80 | 4.77 | 1.42 | 0.31 | 1.25 | 4.80 | 0.47 | 0.29 | 1 |
| 730 | 480218 | AA677457 | 5.16 | 3.62 | 1.42 | 0.74 | 0.72 | 3.28 | 1.18 | 1.20 | 1 |
| 731 | 38393 | R25823 | 10.37 | 7.29 | 1.42 | 1.74 | 1.13 | 0.53 | 0.67 | 3.04 | 1 |
| 732 | 280528 | N47316 | 12.96 | 9.12 | 1.42 | 5.74 | 0.66 | 0.22 | 0.24 | 0.24 | 1 |
| 733 | 252382 | H87108 | 11.19 | 7.88 | 1.42 | 1.40 | 3.07 | 0.53 | 0.60 | 1.48 | 1 |
| 734 | 298779 | N75318 | 1.70 | 1.20 | 1.42 | 0.99 | 0.94 | 3.31 | 0.88 | 0.97 | 1 |
| 735 | 281851 | H99215 | 1.10 | 0.78 | 1.42 | 3.28 | 0.92 | 1.14 | 0.94 | 0.81 | 1 |
| 736 | 32495 | R43485 | 3.20 | 2.26 | 1.42 | 0.72 | 1.02 | 1.09 | 3.22 | 1.04 | 1 |
| 737 | 383706 | AA704332 | 14.70 | 10.38 | 1.42 | 0.67 | 0.81 | 3.89 | 0.79 | 0.93 | 1 |
| 738 | 853809 | AA688470 | 35.90 | 25.35 | 1.42 | 3.32 | 0.52 | 0.33 | 1.27 | 1.63 | 1 |
| 739 | 30275 | R42533 | 0.55 | 0.39 | 1.42 | 4.06 | 0.83 | 1.02 | 0.58 | 0.58 | 1 |
| 740 | 452059 | AA707121 | 3.59 | 2.54 | 1.41 | 0.70 | 0.84 | 3.38 | 0.98 | 1.18 | 1 |
| 741 | 785975 | AA448599 | 1.30 | 0.92 | 1.41 | 1.31 | 1.21 | 0.44 | 0.65 | 3.44 | 1 |
| 742 | 362251 | AA001199 | 4.25 | 3.02 | 1.41 | 0.64 | 0.71 | 3.53 | 1.12 | 1.05 | 1 |
| 743 | 713193 | AA284834 | 3.86 | 2.74 | 1.41 | 0.67 | 0.56 | 4.15 | 0.82 | 0.85 | 1 |
| 744 | 82734 | 173558 | 6.70 | 4.75 | 1.41 | 1.45 | 0.53 | 0.71 | 1.22 | 3.13 | 1 |
| 745 | 814595 | AA480906 | 1.97 | 1.40 | 1.41 | 1.28 | 1.21 | 3.26 | 0.83 | 0.46 | 1 |
| 746 | 281737 | N51740 | 3.44 | 2.44 | 1.41 | 1.01 | 1.01 | 3.38 | 0.82 | 0.82 | 1 |
| 747 | 208985 | H60696 | 1.50 | 1.07 | 1.41 | 0.79 | 1.04 | 3.16 | 1.12 | 0.92 | 1 |
| 748 | 85809 | T62040 | 25.61 | 18.21 | 1.41 | 0.23 | 3.00 | 1.72 | 1.26 | 0.82 | 1 |
| 749 | 247448 | N58052 | 3.88 | 2.76 | 1.41 | 0.86 | 1.01 | 3.16 | 1.09 | 0.91 | 1 |
| 750 | 126847 | R07196 | 58.28 | 41.47 | 1.41 | 0.56 | 0.64 | 3.32 | 1.25 | 1.25 | 1 |
| 751 | 781487 | AA432124 | 2.99 | 2.13 | 1.40 | 3.77 | 1.63 | 0.51 | 0.38 | 0.73 | 1 |
| 752 | 451918 | AA708964 | 7.43 | 5.29 | 1.40 | 0.79 | 0.79 | 3.14 | 1.07 | 1.22 | 1 |
| 753 | 269583 | N24155 | 14.69 | 10.48 | 1.40 | 0.59 | 0.65 | 3.29 | 1.19 | 1.29 | 1 |
| 754 | 814881 | AA481052 | 0.20 | 0.14 | 1.40 | 0.51 | 0.91 | 3.21 | 1.36 | 1.02 | 1 |
| 755 | 208165 | H82529 | 4.21 | 3.01 | 1.40 | 0.74 | 0.90 | 3.59 | 0.95 | 0.82 | 1 |
| 756 | 221778 | 892216 | 47.45 | 33.92 | 1.40 | 0.54 | 0.85 | 3.34 | 1.14 | 1.13 | 1 |
| 757 | 795730 | AA460282 | 0.92 | 0.66 | 1.40 | 1.03 | 0.92 | 0.64 | 3.08 | 1.32 | 1 |
| 758 | 144932 | R78807 | 8.62 | 6.17 | 1.40 | 0.75 | 1.56 | 3.16 | 0.71 | 0.81 | 1 |
| 759 | 219937 | H84759 | 21.24 | 15.21 | 1.40 | 0.60 | 0.60 | 3.27 | 1.27 | 1.24 | 1 |
| 760 | 384134 | AA702193 | 27.27 | 19.57 | 1.39 | 0.63 | 0.74 | 3.62 | 0.84 | 1.14 | 1 |
| 761 | 381054 | AA054439 | 0.13 | 0.09 | 1.39 | 0.89 | 1.39 | 3.10 | 0.80 | 0.78 | 1 |
| 762 | 898058 | AA598775 | 1.84 | 1.32 | 1.39 | 0.75 | 0.89 | 3.47 | 0.80 | 1.05 | 1 |
| 763 | 219881 | H81716 | 48.51 | 34.84 | 1.39 | 0.63 | 0.76 | 3.60 | 0.97 | 1.01 | 1 |
| 764 | 208225 | H65300 | 72.84 | 52.40 | 1.39 | 0.49 | 0.80 | 3.47 | 1.08 | 1.12 | 1 |
| 765 | 152270 | H04757 | 1.43 | 1.03 | 1.39 | 0.97 | 1.01 | 3.24 | 0.63 | 1.08 | 1 |
| 766 | 25398 | R17747 | 0.59 | 0.42 | 1.39 | 3.88 | 1.18 | 0.52 | 0.61 | 0.75 | 1 |
| 767 | 431888 | AA678361 | 1.40 | 1.01 | 1.38 | 0.95 | 0.95 | 3.07 | 1.29 | 0.66 | 1 |
| 768 | 138255 | R58840 | 2.88 | 2.08 | 1.38 | 0.97 | 0.84 | 3.29 | 0.79 | 1.03 | 1 |
| 769 | 123474 | R00707 | 3.34 | 2.42 | 1.38 | 0.49 | 1.18 | 0.69 | 0.50 | 4.05 | 1 |
| 770 | 431269 | AA682585 | 1.17 | 0.85 | 1.38 | 0.62 | 1.10 | 3.20 | 0.78 | 1.21 | 1 |
| 771 | 309081 | N92895 | 1.76 | 1.27 | 1.38 | 0.45 | 1.40 | 4.28 | 0.39 | 0.38 | 1 |
| 772 | 33854 | R44837 | 1.79 | 1.29 | 1.38 | 3.51 | 0.79 | 0.24 | 1.25 | 1.10 | 1 |
| 773 | 769857 | AA430367 | 2.79 | 2.02 | 1.38 | 1.10 | 0.53 | 1.22 | 3.57 | 0.48 | 1 |
| 774 | 769947 | AA430410 | 1.01 | 0.73 | 1.38 | 0.61 | 0.83 | 3.68 | 0.77 | 1.00 | 1 |
| 775 | 34888 | R19878 | 0.71 | 0.52 | 1.38 | 3.81 | 0.73 | 0.90 | 0.68 | 0.78 | 1 |
| 776 | 451161 | AA704749 | 0.78 | 0.57 | 1.37 | 0.66 | 0.92 | 3.06 | 0.64 | 1.58 | 1 |
| 777 | 230236 | H94870 | 140.80 | 102.66 | 1.37 | 0.57 | 0.75 | 3.14 | 1.30 | 1.10 | 1 |
| 778 | 1292829 | AA776702 | 0.13 | 0.10 | 1.37 | 0.85 | 1.31 | 0.91 | 0.11 | 3.68 | 1 |
| 779 | 288683 | N62394 | 17.85 | 13.04 | 1.37 | 0.36 | 3.06 | 1.83 | 1.08 | 0.72 | 1 |
| 780 | 450880 | AA682842 | 2.29 | 1.68 | 1.37 | 0.48 | 0.84 | 3.92 | 0.93 | 0.68 | 1 |
| 781 | 782501 | AA431772 | 0.98 | 0.70 | 1.37 | 0.80 | 1.03 | 3.07 | 1.22 | 0.71 | 1 |
| 782 | 141854 | R70598 | 4.52 | 3.31 | 1.37 | 0.83 | 0.79 | 3.26 | 1.09 | 0.87 | 1 |
| 783 | 704076 | AA279172 | 18.54 | 13.58 | 1.37 | 0.72 | 0.82 | 3.06 | 0.94 | 1.28 | 1 |
| 784 | 1048696 | AA620608 | 2.14 | 1.57 | 1.36 | 3.05 | 0.86 | 0.35 | 0.65 | 1.90 | 1 |
| 785 | 809488 | AA443118 | 1.25 | 0.92 | 1.36 | 3.53 | 0.88 | 0.82 | 0.88 | 0.70 | 1 |
| 786 | 725384 | AA291972 | 10.64 | 7.82 | 1.36 | 0.26 | 0.95 | 3.09 | 1.77 | 0.73 | 1 |
| 787 | 773204 | AA425892 | 35.81 | 26.33 | 1.36 | 0.50 | 3.17 | 1.74 | 0.39 | 1.00 | 1 |
| 788 | 813861 | AA447764 | 3.53 | 2.60 | 1.36 | 0.77 | 3.37 | 1.13 | 0.70 | 0.83 | 1 |
| 789 | 770879 | AA434403 | 1.86 | 1.37 | 1.36 | 0.64 | 0.74 | 0.38 | 4.06 | 0.98 | 1 |
| 790 | 769673 | AA428341 | 3.15 | 2.32 | 1.36 | 1.20 | 0.77 | 3.03 | 1.15 | 0.64 | 1 |
| 791 | 221776 | H92215 | 25.91 | 19.13 | 1.35 | 0.50 | 0.71 | 3.35 | 0.93 | 1.28 | 1 |
| 792 | 814744 | AA454925 | 33.12 | 24.47 | 1.35 | 0.54 | 0.78 | 3.07 | 1.24 | 1.14 | 1 |
| 793 | 688128 | AA233549 | 0.08 | 0.06 | 1.35 | 3.60 | 1.27 | 0.57 | 0.47 | 0.86 | 1 |
| 794 | 273048 | N36402 | 2.18 | 1.61 | 1.35 | 1.11 | 1.06 | 0.53 | 0.89 | 3.18 | 1 |
| 795 | 223314 | H88275 | 0.17 | 0.13 | 1.35 | 0.69 | 0.97 | 3.38 | 0.31 | 1.40 | 1 |
| 796 | 392673 | AA708348 | 69.06 | 51.24 | 1.35 | 0.46 | 0.64 | 3.07 | 1.39 | 1.18 | 1 |
| 797 | 1384851 | AA857496 | 0.91 | 0.67 | 1.34 | 0.59 | 0.86 | 4.54 | 0.30 | 0.43 | 1 |
| 798 | 35010 | R45118 | 1.58 | 1.16 | 1.34 | 0.43 | 1.71 | 3.54 | 0.57 | 0.47 | 1 |
| 799 | 248734 | 879078 | 7.73 | 5.76 | 1.34 | 0.71 | 0.86 | 3.19 | 0.87 | 1.09 | 1 |
| 800 | 889387 | AA253442 | 2.00 | 1.49 | 1.34 | 3.41 | 0.95 | 0.91 | 0.60 | 0.84 | 1 |
| 801 | 231718 | H92875 | 0.92 | 0.89 | 1.34 | 0.49 | 0.77 | 3.16 | 1.73 | 0.54 | 1 |
| 802 | 322857 | W44938 | 0.49 | 0.37 | 1.34 | 0.78 | 0.83 | 3.07 | 0.99 | 1.01 | 1 |
| 803 | 480164 | AA676920 | 3.38 | 2.54 | 1.33 | 0.35 | 0.57 | 4.83 | 0.58 | 0.33 | 1 |
| 804 | 322138 | W37532 | 105.05 | 78.95 | 1.33 | 0.46 | 0.52 | 3.01 | 1.47 | 1.20 | 1 |
| 805 | 823562 | AA497111 | 2.57 | 1.93 | 1.33 | 0.52 | 3.32 | 0.34 | 0.62 | 1.85 | 1 |
| 806 | 211387 | H68875 | 35.54 | 28.74 | 1.33 | 0.67 | 0.61 | 3.50 | 0.82 | 1.05 | 1 |
| 807 | 48518 | H09172 | 0.70 | 0.52 | 1.33 | 1.04 | 0.92 | 0.87 | 1.00 | 3.01 | 1 |
| 808 | 450300 | AA882838 | 6.02 | 4.55 | 1.32 | 0.41 | 0.60 | 3.45 | 1.01 | 1.14 | 1 |
| 809 | 431029 | AA758470 | 0.13 | 0.10 | 1.32 | 1.19 | 1.00 | 3.28 | 0.51 | 0.65 | 1 |
| 810 | 781492 | AA432134 | 0.51 | 0.33 | 1.32 | 0.85 | 0.75 | 0.86 | 1.02 | 3.11 | 1 |
| 811 | 782687 | AA447592 | 0.83 | 0.63 | 1.32 | 0.80 | 0.68 | 0.97 | 3.43 | 0.70 | 1 |
| 812 | 767405 | AA417921 | 3.01 | 2.29 | 1.31 | 3.14 | 0.91 | 0.45 | 1.10 | 0.96 | 1 |
| 813 | 1472689 | AA873159 | 14.58 | 11.10 | 1.31 | 4.38 | 0.54 | 0.51 | 0.55 | 0.59 | 1 |
| 814 | 725224 | AA401828 | 0.84 | 0.64 | 1.31 | 0.42 | 0.80 | 3.88 | 0.82 | 0.68 | 1 |
| 815 | 810109 | AA484979 | 1.46 | 1.12 | 1.31 | 1.19 | 0.70 | 0.48 | 0.77 | 3.43 | 1 |
| 816 | 825368 | AA504505 | 0.65 | 0.50 | 1.31 | 0.77 | 0.73 | 3.04 | 1.13 | 0.88 | 1 |
| 817 | 1558655 | AA976581 | 7.73 | 5.91 | 1.31 | 0.71 | 0.54 | 3.63 | 0.95 | 0.72 | 1 |
| 818 | 481383 | AA704908 | 15.65 | 11.97 | 1.31 | 0.56 | 0.70 | 3.40 | 1.05 | 0.83 | 1 |
| 819 | 1552618 | AA927544 | 0.45 | 0.34 | 1.31 | 3.13 | 0.44 | 0.50 | 0.97 | 1.48 | 1 |
| 820 | 840268 | AA485303 | 14.99 | 11.49 | 1.30 | 0.25 | 4.33 | 0.54 | 0.45 | 0.95 | 1 |
| 821 | 454469 | AA677337 | 4.99 | 3.82 | 1.30 | 0.61 | 0.84 | 3.16 | 1.19 | 0.91 | 1 |
| 822 | 212473 | H69553 | 13.13 | 10.08 | 1.30 | 0.63 | 0.70 | 3.49 | 0.85 | 0.84 | 1 |
| 823 | 80109 | T63324 | 5.71 | 4.38 | 1.30 | 3.68 | 0.73 | 0.23 | 0.76 | 1.13 | 1 |
| 824 | 770059 | AA427561 | 1.46 | 1.12 | 1.30 | 0.68 | 0.58 | 3.39 | 1.33 | 0.55 | 1 |
| 825 | 123278 | R00283 | 2.88 | 2.21 | 1.30 | 3.04 | 0.81 | 0.89 | 0.73 | 1.02 | 1 |
| 826 | 289582 | N62763 | 48.29 | 37.28 | 1.30 | 0.63 | 0.69 | 3.01 | 1.01 | 1.15 | 1 |
| 827 | 47481 | H11732 | 2.68 | 2.06 | 1.29 | 3.14 | 0.93 | 0.31 | 0.95 | 1.13 | 1 |
| 828 | 435128 | AA701328 | 12.02 | 9.31 | 1.29 | 0.54 | 0.87 | 3.25 | 0.96 | 1.03 | 1 |
| 829 | 595148 | AA164836 | 1.19 | 0.93 | 1.29 | 3.76 | 0.84 | 0.82 | 0.82 | 0.80 | 1 |
| 830 | 1049030 | AA778683 | 1.11 | 0.88 | 1.29 | 0.78 | 0.80 | 3.04 | 0.99 | 0.82 | 1 |
| 831 | 49499 | H15549 | 1.40 | 1.09 | 1.28 | 3.02 | 1.14 | 0.67 | 0.61 | 0.98 | 1 |
| 832 | 756554 | AA481437 | 1.47 | 1.15 | 1.28 | 0.83 | 0.81 | 3.40 | 1.18 | 0.60 | 1 |
| 833 | 383752 | AA704370 | 4.47 | 3.49 | 1.28 | 0.74 | 0.62 | 3.01 | 1.17 | 0.87 | 1 |
| 834 | 845079 | AA197334 | 46.13 | 35.99 | 1.28 | 0.60 | 0.83 | 3.00 | 1.02 | 1.16 | 1 |
| 835 | 83541 | T89593 | 0.83 | 0.65 | 1.28 | 0.99 | 0.69 | 3.29 | 0.68 | 0.75 | 1 |
| 836 | 703918 | AA279080 | 2.86 | 2.08 | 1.27 | 3.10 | 1.39 | 0.21 | 0.48 | 1.20 | 1 |
| 837 | 815835 | AA485249 | 0.26 | 0.21 | 1.27 | 0.51 | 0.68 | 3.48 | 0.68 | 0.86 | 1 |
| 838 | 280897 | N47443 | 3.87 | 2.88 | 1.27 | 0.51 | 1.08 | 3.77 | 0.57 | 0.43 | 1 |
| 839 | 399284 | AA774501 | 0.40 | 0.32 | 1.27 | 0.50 | 0.74 | 3.32 | 0.91 | 0.87 | 1 |
| 840 | 588915 | AA157813 | 12.44 | 9.82 | 1.27 | 3.13 | 0.66 | 0.57 | 0.78 | 1.20 | 1 |
| 841 | 140299 | R68923 | 29.84 | 23.43 | 1.27 | 0.61 | 0.62 | 3.06 | 0.86 | 1.15 | 1 |
| 842 | 454795 | AA877300 | 2.68 | 2.13 | 1.26 | 0.55 | 0.77 | 3.87 | 0.41 | 0.69 | 1 |
| 843 | 756450 | AA482127 | 1.60 | 1.28 | 1.26 | 0.59 | 0.75 | 3.30 | 1.03 | 0.62 | 1 |
| 844 | 280982 | N50859 | 2.72 | 2.18 | 1.26 | 0.79 | 0.58 | 3.32 | 0.76 | 0.85 | 1 |
| 845 | 295454 | N78088 | 22.29 | 17.73 | 1.26 | 0.68 | 0.60 | 3.06 | 0.87 | 1.08 | 1 |
| 846 | 841057 | AA488765 | 1.01 | 0.80 | 1.26 | 0.50 | 1.34 | 3.30 | 0.44 | 0.70 | 1 |
| 847 | 248831 | N59532 | 2.15 | 1.72 | 1.25 | 0.62 | 3.67 | 0.93 | 0.36 | 0.68 | 1 |
| 848 | 136026 | R34225 | 5.25 | 4.19 | 1.25 | 3.84 | 0.92 | 0.34 | 0.40 | 0.76 | 1 |
| 849 | 824270 | AA491261 | 1.84 | 1.47 | 1.25 | 1.04 | 0.66 | 3.03 | 0.61 | 0.91 | 1 |
| 850 | 451598 | AA707080 | 6.37 | 5.09 | 1.25 | 0.41 | 0.68 | 3.25 | 0.94 | 0.96 | 1 |
| 851 | 883274 | AA213667 | 2.82 | 2.25 | 1.25 | 0.79 | 0.60 | 3.20 | 1.05 | 0.60 | 1 |
| 852 | 223180 | H86559 | 18.89 | 15.13 | 1.25 | 0.56 | 0.64 | 3.28 | 0.88 | 0.88 | 1 |
| 853 | 788308 | AA451863 | 2.81 | 2.25 | 1.25 | 3.33 | 0.84 | 0.40 | 0.89 | 0.76 | 1 |
| 854 | 196189 | H92281 | 23.31 | 18.72 | 1.25 | 0.52 | 0.63 | 0.70 | 1.34 | 3.03 | 1 |
| 855 | 811914 | AA454988 | 4.71 | 3.79 | 1.24 | 1.32 | 0.89 | 0.63 | 0.27 | 3.12 | 1 |
| 856 | 815090 | AA485194 | 1.89 | 1.52 | 1.24 | 3.17 | 0.68 | 0.71 | 0.79 | 0.67 | 1 |
| 857 | 203350 | H54364 | 1.68 | 1.35 | 1.24 | 3.05 | 0.96 | 0.72 | 0.79 | 0.68 | 1 |
| 858 | 361943 | AA001444 | 6.41 | 5.17 | 1.24 | 0.49 | 1.71 | 0.29 | 0.70 | 3.01 | 1 |
| 859 | 260168 | N32071 | 0.11 | 0.09 | 1.24 | 0.21 | 1.44 | 0.54 | 0.00 | 4.02 | 1 |
| 860 | 1404995 | AA845584 | 0.04 | 0.03 | 1.24 | 0.00 | 3.16 | 1.20 | 1.85 | 0.00 | 1 |
| 861 | 773284 | AA425298 | 0.07 | 0.06 | 1.24 | 3.14 | 1.07 | 0.17 | 0.51 | 1.30 | 1 |
| 862 | 435953 | AA701976 | 3.57 | 2.89 | 1.24 | 0.40 | 1.12 | 3.20 | 0.73 | 0.73 | 1 |
| 863 | 480143 | AA676865 | 3.02 | 2.45 | 1.24 | 0.63 | 0.80 | 3.03 | 0.73 | 0.98 | 1 |
| 864 | 52730 | H29500 | 13.72 | 11.12 | 1.23 | 3.10 | 0.59 | 0.69 | 0.61 | 1.18 | 1 |
| 865 | 270331 | N29457 | 21.84 | 17.78 | 1.23 | 0.53 | 0.66 | 3.14 | 0.70 | 1.10 | 1 |
| 866 | 40838 | R55985 | 5.30 | 4.32 | 1.23 | 0.24 | 0.74 | 0.34 | 0.98 | 3.83 | 1 |
| 867 | 148838 | H13438 | 11.57 | 9.45 | 1.22 | 0.67 | 0.74 | 3.07 | 0.82 | 0.82 | 1 |
| 868 | 396085 | AA757711 | 4.80 | 3.92 | 1.22 | 0.49 | 0.74 | 3.30 | 0.81 | 0.78 | 1 |
| 869 | 435598 | AA703159 | 0.71 | 0.58 | 1.22 | 0.63 | 0.84 | 3.01 | 0.88 | 0.75 | 1 |
| 870 | 190892 | H38848 | 6.03 | 4.94 | 1.22 | 0.54 | 1.01 | 3.14 | 0.67 | 0.74 | 1 |
| 871 | 825234 | AA504132 | 17.18 | 14.12 | 1.22 | 0.52 | 0.63 | 3.28 | 0.84 | 0.82 | 1 |
| 872 | 268455 | N21688 | 37.89 | 31.16 | 1.22 | 0.60 | 0.66 | 3.31 | 0.68 | 0.83 | 1 |
| 873 | 1623328 | A1015196 | 6.84 | 5.54 | 1.20 | 1.11 | 0.99 | 0.38 | 0.45 | 3.05 | 1 |
| 874 | 138070 | R34273 | 9.69 | 8.11 | 1.19 | 3.12 | 0.74 | 0.25 | 0.52 | 1.34 | 1 |
| 875 | 450997 | AA704278 | 3.53 | 2.98 | 1.19 | 0.62 | 0.64 | 3.19 | 0.84 | 0.68 | 1 |
| 876 | 433300 | AA699719 | 7.79 | 6.54 | 1.19 | 0.51 | 0.57 | 3.05 | 0.78 | 1.04 | 1 |
| 877 | 53158 | R16146 | 1.10 | 0.95 | 1.16 | 3.33 | 0.63 | 0.51 | 0.63 | 0.69 | 1 |
| 878 | 814053 | AA485495 | 17.56 | 15.23 | 1.15 | 3.28 | 0.85 | 0.32 | 0.08 | 1.25 | 1 |
| 879 | 123755 | R01304 | 4.23 | 3.68 | 1.15 | 0.29 | 1.11 | 3.95 | 0.22 | 0.20 | 1 |
| 880 | 46843 | H10072 | 1.44 | 1.25 | 1.15 | 0.44 | 0.96 | 3.26 | 0.65 | 0.44 | 1 |
| 881 | 754280 | AA479284 | 15.02 | 13.05 | 1.15 | 3.99 | 0.73 | 0.24 | 0.21 | 0.58 | 1 |
| 882 | 745490 | AA625979 | 3.01 | 2.83 | 1.15 | 0.30 | 1.12 | 3.29 | 0.68 | 0.32 | 1 |
| 883 | 141361 | R64454 | 1.36 | 1.20 | 1.14 | 3.54 | 1.05 | 0.30 | 0.55 | 0.23 | 1 |
| 884 | 80050 | T63245 | 34.68 | 30.90 | 1.12 | 0.22 | 1.07 | 3.25 | 0.55 | 0.52 | 1 |
| 885 | 489076 | AA057195 | 0.03 | 0.02 | 1.11 | 0.00 | 3.69 | 1.88 | 0.00 | 0.00 | 1 |
| 886 | 504358 | AA131885 | 0.02 | 0.02 | 1.10 | 0.45 | 3.93 | 0.37 | 0.14 | 0.59 | 1 |
| 887 | 826995 | AA521384 | 2.31 | 2.14 | 1.08 | 0.77 | 0.36 | 0.54 | 3.02 | 0.72 | 1 |
| 888 | 279800 | N49107 | 8.14 | 7.59 | 1.07 | 0.44 | 0.49 | 0.20 | 1.04 | 3.18 | 1 |
| 889 | 745347 | AA625666 | 7.22 | 8.99 | 1.03 | 0.73 | 0.57 | 0.32 | 0.45 | 3.09 | 1 |
| 890 | 788602 | AA478470 | 10.45 | 10.12 | 1.03 | 3.01 | 0.68 | 0.30 | 0.55 | 0.62 | 1 |
| 891 | 306743 | N91787 | 10.70 | 11.01 | 0.97 | 0.77 | 0.34 | 0.11 | 0.82 | 3.03 | 1 |

TABLE 2-8
Good vs. Poor Clinical outcome: Genes upregulated 3 fold or more in at least 2 tumor samples.

| | | Gen Bank | | | Fold(AveGood/ | Fold(mpm27/Ave | Fold(mpm28/Ave | Fold(mpm29/Ave | Fold(mpm30/Ave | Fold(mpm35/Ave | # of Tumors |
|---|---|---|---|---|---|---|---|---|---|---|---|
| Order | IMAGE Clone ID | Accession Number | AveGood | AvePoor | AvePoor) | Poor) | Poor) | Poor) | Poor) | Poor) | Samples |
| 1 | 299679 | W05628 | 2.55 | 0.50 | 5.07 | 7.07 | 6.80 | 3.51 | 0.68 | 7.30 | 4 |
| 2 | 811162 | AA486471 | 28.87 | 6.85 | 4.21 | 0.70 | 3.61 | 6.33 | 4.17 | 6.26 | 4 |
| 3 | 471641 | AA034945 | 11.03 | 1.08 | 10.17 | 21.19 | 2.20 | 4.22 | 2.10 | 21.15 | 3 |
| 4 | 281039 | N47717 | 36.67 | 4.42 | 8.29 | 22.87 | 1.40 | 0.27 | 9.85 | 7.08 | 3 |
| 5 | 282089 | N48259 | 27.13 | 3.45 | 7.87 | 22.41 | 0.41 | 0.67 | 7.05 | 8.80 | 3 |
| 6 | 155072 | R71393 | 3.32 | 0.45 | 7.44 | 8.50 | 1.97 | 1.30 | 9.52 | 15.90 | 3 |
| 7 | 208210 | H65286 | 2.70 | 0.42 | 6.48 | 14.00 | 1.98 | 0.98 | 5.30 | 10.17 | 3 |
| 8 | 770066 | AA430545 | 26.72 | 4.62 | 5.78 | 12.05 | 0.67 | 1.90 | 8.30 | 5.96 | 3 |
| 9 | 712950 | AA282273 | 38.22 | 6.94 | 5.50 | 8.84 | 1.04 | 0.35 | 4.37 | 12.92 | 3 |
| 10 | 81331 | T60111 | 4.13 | 0.82 | 5.03 | 11.18 | 1.30 | 0.80 | 8.59 | 3.28 | 3 |
| 11 | 287728 | N59158 | 3.49 | 0.71 | 4.90 | 14.39 | 0.35 | 0.46 | 3.86 | 5.42 | 3 |
| 12 | 41228 | R58953 | 0.05 | 0.01 | 4.38 | 9.07 | 2.53 | 4.46 | 2.14 | 3.71 | 3 |
| 13 | 122345 | T99191 | 0.03 | 0.01 | 4.16 | 12.66 | 4.17 | 0.79 | 0.00 | 3.18 | 3 |
| 14 | 186918 | H43317 | 6.27 | 1.60 | 3.93 | 3.28 | 1.41 | 1.33 | 4.30 | 9.31 | 3 |
| 15 | 1049033 | AA778675 | 2.50 | 0.65 | 3.87 | 4.86 | 2.38 | 4.54 | 2.55 | 5.03 | 3 |
| 16 | 195365 | R88904 | 1.81 | 0.50 | 3.62 | 6.03 | 1.45 | 0.88 | 3.51 | 6.23 | 3 |
| 17 | 50114 | H16743 | 1.48 | 0.43 | 3.43 | 5.36 | 4.18 | 4.79 | 2.28 | 0.54 | 3 |
| 18 | 838611 | AA456975 | 87.41 | 25.99 | 3.36 | 5.71 | 4.22 | 4.22 | 1.32 | 1.35 | 3 |
| 19 | 283196 | N45282 | 5.16 | 1.55 | 3.32 | 7.31 | 0.87 | 1.42 | 3.24 | 3.77 | 3 |
| 20 | 745525 | AA626248 | 4.16 | 1.27 | 3.28 | 5.86 | 1.71 | 0.34 | 4.76 | 3.74 | 3 |
| 21 | 159487 | H15926 | 0.12 | 0.04 | 2.92 | 2.50 | 3.69 | 3.70 | 1.59 | 3.11 | 3 |
| 22 | 1034473 | AA779728 | 76.12 | 27.06 | 2.81 | 2.22 | 3.21 | 3.87 | 0.79 | 3.97 | 3 |
| 23 | 138444 | R68272 | 2.02 | 0.74 | 2.75 | 4.16 | 1.66 | 1.35 | 3.32 | 3.28 | 3 |
| 24 | 378488 | AA777187 | 17.01 | 7.17 | 2.37 | 3.23 | 1.20 | 0.99 | 3.13 | 3.31 | 3 |
| 25 | 767130 | AA424535 | 4.28 | 0.37 | 11.66 | 1.60 | 25.69 | 28.44 | 1.49 | 1.02 | 2 |
| 26 | 307337 | N95226 | 10.81 | 0.96 | 11.29 | 1.65 | 7.47 | 44.45 | 1.85 | 1.22 | 2 |
| 27 | 502625 | AA134576 | 7.27 | 0.90 | 8.08 | 1.44 | 1.15 | 33.58 | 1.04 | 3.17 | 2 |
| 28 | 41869 | R66438 | 1.41 | 0.28 | 5.03 | 1.46 | 5.93 | 15.74 | 1.30 | 0.71 | 2 |
| 29 | 301735 | N90882 | 4.10 | 0.90 | 4.56 | 9.05 | 2.75 | 2.93 | 0.99 | 7.09 | 2 |
| 30 | 136605 | R35051 | 32.22 | 7.39 | 4.36 | 2.55 | 3.53 | 11.13 | 1.94 | 2.65 | 2 |
| 31 | 362278 | AA001219 | 5.53 | 1.41 | 3.91 | 2.64 | 2.34 | 0.78 | 6.56 | 7.23 | 2 |
| 32 | 756708 | AA443903 | 2.58 | 0.67 | 3.84 | 0.50 | 4.59 | 10.09 | 2.99 | 1.01 | 2 |
| 33 | 265028 | N21084 | 1.22 | 0.32 | 3.78 | 1.00 | 4.36 | 11.96 | 0.59 | 0.98 | 2 |
| 34 | 1409509 | AA868929 | 2.84 | 0.75 | 3.78 | 0.36 | 5.86 | 11.75 | 0.59 | 0.31 | 2 |
| 35 | 365707 | AA025434 | 1.94 | 0.53 | 3.68 | 0.58 | 3.36 | 10.11 | 1.36 | 3.00 | 2 |
| 36 | 768953 | AA424920 | 3.43 | 0.94 | 3.66 | 0.96 | 5.41 | 10.03 | 0.93 | 0.97 | 2 |
| 37 | 357628 | W94121 | 1.80 | 0.49 | 3.65 | 5.62 | 0.55 | 0.68 | 2.00 | 9.41 | 2 |
| 38 | 35058 | R45192 | 28.75 | 7.90 | 3.64 | 9.19 | 4.22 | 2.07 | 1.34 | 1.38 | 2 |
| 39 | 145740 | R77948 | 30.23 | 8.55 | 3.53 | 1.47 | 0.52 | 0.73 | 5.51 | 9.44 | 2 |
| 40 | 757200 | AA443987 | 0.08 | 0.02 | 3.51 | 1.63 | 2.44 | 1.32 | 3.97 | 8.17 | 2 |
| 41 | 856575 | AA633658 | 13.79 | 4.11 | 3.36 | 1.01 | 1.91 | 2.38 | 6.90 | 4.57 | 2 |
| 42 | 731469 | AA412417 | 3.83 | 1.14 | 3.35 | 3.72 | 2.54 | 5.00 | 2.74 | 2.75 | 2 |
| 43 | 140197 | R66101 | 2.60 | 0.79 | 3.29 | 0.86 | 3.45 | 10.28 | 0.99 | 0.88 | 2 |
| 44 | 46166 | H09076 | 12.10 | 3.68 | 3.29 | 8.38 | 1.09 | 0.51 | 2.55 | 3.90 | 2 |
| 45 | 123264 | R00276 | 8.05 | 2.46 | 3.28 | 0.59 | 4.91 | 1.61 | 1.60 | 7.68 | 2 |
| 46 | 37980 | R61372 | 0.83 | 0.25 | 3.26 | 8.99 | 3.72 | 2.26 | 0.96 | 0.35 | 2 |
| 47 | 1055707 | AA828113 | 3.79 | 1.18 | 3.23 | 6.07 | 2.96 | 1.26 | 2.50 | 3.34 | 2 |
| 48 | 245277 | N53447 | 7.05 | 2.22 | 3.18 | 8.02 | 1.26 | 0.59 | 2.35 | 3.69 | 2 |
| 49 | 878596 | AA775257 | 5.91 | 1.88 | 3.14 | 7.62 | 1.48 | 0.91 | 2.42 | 3.25 | 2 |
| 50 | 66599 | T67128 | 3.06 | 0.98 | 3.12 | 3.86 | 0.81 | 0.45 | 1.32 | 9.18 | 2 |
| 51 | 782335 | AA432270 | 4.04 | 1.30 | 3.12 | 6.21 | 5.84 | 1.80 | 0.66 | 1.08 | 2 |
| 52 | 193087 | H47076 | 0.06 | 0.02 | 3.11 | 3.68 | 0.71 | 1.77 | 0.49 | 8.89 | 2 |
| 53 | 151477 | H02837 | 2.32 | 0.75 | 3.09 | 10.04 | 0.89 | 0.57 | 3.20 | 0.75 | 2 |
| 54 | 768020 | AA418748 | 4.17 | 1.36 | 3.06 | 0.74 | 4.10 | 0.74 | 0.60 | 9.12 | 2 |
| 55 | 35329 | R45517 | 1.82 | 0.64 | 2.86 | 3.39 | 2.99 | 4.46 | 1.70 | 1.75 | 2 |
| 56 | 245990 | N55459 | 101.29 | 36.00 | 2.81 | 0.45 | 1.13 | 0.94 | 6.81 | 4.74 | 2 |
| 57 | 288983 | N59816 | 2.12 | 0.75 | 2.81 | 0.82 | 1.41 | 1.52 | 4.53 | 5.77 | 2 |
| 58 | 646037 | AA196979 | 98.80 | 35.15 | 2.81 | 0.92 | 6.21 | 3.16 | 1.58 | 2.19 | 2 |
| 59 | 434952 | AA700680 | 1.23 | 0.45 | 2.77 | 1.83 | 3.75 | 6.22 | 0.82 | 1.24 | 2 |
| 60 | 231574 | H92821 | 2.29 | 0.83 | 2.75 | 6.94 | 3.46 | 1.02 | 0.85 | 1.47 | 2 |
| 61 | 840590 | AA487902 | 48.94 | 17.94 | 2.73 | 4.81 | 1.98 | 0.79 | 1.17 | 4.89 | 2 |
| 62 | 78353 | T56281 | 39.72 | 14.62 | 2.72 | 0.58 | 0.96 | 1.14 | 6.48 | 4.41 | 2 |
| 63 | 1418621 | AA878120 | 3.31 | 1.22 | 2.71 | 4.87 | 1.25 | 1.55 | 2.55 | 3.34 | 2 |
| 64 | 66902 | T69477 | 2.93 | 1.09 | 2.68 | 0.63 | 4.28 | 7.31 | 0.46 | 0.72 | 2 |
| 65 | 823655 | AA498988 | 6.96 | 2.61 | 2.67 | 3.01 | 2.02 | 4.22 | 2.85 | 1.24 | 2 |
| 66 | 246430 | N53031 | 1.50 | 0.57 | 2.63 | 3.32 | 0.54 | 7.85 | 0.69 | 0.76 | 2 |
| 67 | 491751 | AA150500 | 2.62 | 1.00 | 2.62 | 3.79 | 1.22 | 1.62 | 1.56 | 4.89 | 2 |
| 68 | 82905 | T69305 | 3.92 | 1.50 | 2.62 | 3.41 | 2.68 | 1.71 | 3.01 | 2.27 | 2 |
| 69 | 346583 | W74533 | 3.04 | 1.17 | 2.60 | 5.42 | 1.46 | 0.54 | 2.18 | 3.42 | 2 |
| 70 | 298417 | N74131 | 6.38 | 2.46 | 2.60 | 1.63 | 0.37 | 0.65 | 4.00 | 6.33 | 2 |
| 71 | 51178 | H17121 | 2.44 | 0.94 | 2.59 | 3.54 | 1.13 | 6.17 | 1.04 | 1.06 | 2 |
| 72 | 447416 | AA702335 | 4.21 | 1.63 | 2.59 | 2.51 | 0.86 | 5.37 | 3.15 | 1.03 | 2 |
| 73 | 42872 | R61883 | 0.81 | 0.32 | 2.55 | 2.17 | 1.20 | 2.58 | 3.69 | 3.11 | 2 |
| 74 | 232772 | H72722 | 39.31 | 15.73 | 2.50 | 0.44 | 0.93 | 1.09 | 5.65 | 4.38 | 2 |
| 75 | 277173 | N34316 | 19.49 | 7.91 | 2.46 | 1.63 | 3.19 | 5.67 | 1.21 | 0.61 | 2 |
| 76 | 795425 | AA453526 | 1.87 | 0.77 | 2.44 | 0.43 | 4.91 | 5.03 | 1.21 | 0.63 | 2 |
| 77 | 743868 | AA634431 | 3.37 | 1.38 | 2.44 | 0.61 | 3.72 | 2.12 | 2.49 | 3.26 | 2 |
| 78 | 40100 | R54590 | 1.16 | 0.48 | 2.41 | 3.24 | 3.10 | 1.95 | 1.76 | 1.99 | 2 |
| 79 | 202535 | H53340 | 52.42 | 21.78 | 2.41 | 0.31 | 0.53 | 0.61 | 6.54 | 4.04 | 2 |
| 80 | 267546 | N23139 | 1.37 | 0.57 | 2.41 | 0.80 | 3.38 | 5.61 | 1.13 | 1.12 | 2 |
| 81 | 740604 | AA479795 | 3.61 | 1.52 | 2.38 | 3.47 | 2.03 | 1.56 | 1.58 | 3.25 | 2 |
| 82 | 214441 | H73590 | 3.93 | 1.66 | 2.37 | 0.70 | 3.78 | 0.22 | 0.28 | 6.89 | 2 |
| 83 | 364022 | AA021586 | 3.15 | 1.33 | 2.36 | 3.36 | 1.42 | 0.76 | 2.42 | 3.86 | 2 |
| 84 | 295985 | N67039 | 0.77 | 0.33 | 2.32 | 3.94 | 1.57 | 1.42 | 3.44 | 1.24 | 2 |
| 85 | 782575 | AA447522 | 2.41 | 1.04 | 2.31 | 3.32 | 0.44 | 0.53 | 2.96 | 4.33 | 2 |
| 86 | 45852 | H08862 | 0.84 | 0.37 | 2.30 | 3.12 | 1.55 | 1.05 | 3.80 | 1.96 | 2 |
| 87 | 66560 | T67053 | 15.54 | 6.85 | 2.27 | 4.65 | 2.10 | 0.08 | 0.18 | 4.34 | 2 |
| 88 | 284383 | N52151 | 2.85 | 1.26 | 2.28 | 2.81 | 0.65 | 0.30 | 4.10 | 3.45 | 2 |
| 89 | 813151 | AA456299 | 1.57 | 0.70 | 2.23 | 4.73 | 1.49 | 3.31 | 0.74 | 0.90 | 2 |
| 90 | 813179 | AA456321 | 4.78 | 2.14 | 2.23 | 3.34 | 0.83 | 0.42 | 4.69 | 1.87 | 2 |
| 91 | 855745 | AA663981 | 28.11 | 12.67 | 2.22 | 0.76 | 5.59 | 0.11 | 0.24 | 4.40 | 2 |
| 92 | 1605539 | AA988345 | 12.27 | 5.54 | 2.22 | 0.56 | 1.01 | 0.91 | 4.70 | 3.90 | 2 |
| 93 | 144747 | R76247 | 3.07 | 1.40 | 2.20 | 3.06 | 0.82 | 2.15 | 3.63 | 1.32 | 2 |
| 94 | 50772 | H16803 | 37.52 | 17.15 | 2.19 | 3.40 | 0.78 | 0.45 | 0.51 | 5.80 | 2 |
| 95 | 214162 | H77766 | 34.05 | 15.57 | 2.19 | 0.17 | 0.51 | 0.43 | 5.78 | 4.05 | 2 |
| 96 | 247117 | N57872 | 29.18 | 13.39 | 2.18 | 0.32 | 0.52 | 0.59 | 5.39 | 4.08 | 2 |
| 97 | 1468260 | AA884926 | 6.47 | 2.98 | 2.17 | 1.03 | 4.41 | 0.25 | 0.23 | 4.96 | 2 |
| 98 | 179083 | H50086 | 0.52 | 0.24 | 2.17 | 3.28 | 0.97 | 1.63 | 1.06 | 3.89 | 2 |
| 99 | 770868 | AA434487 | 3.35 | 1.57 | 2.14 | 4.01 | 3.49 | 0.97 | 0.71 | 1.53 | 2 |
| 100 | 129585 | R16596 | 22.38 | 10.50 | 2.13 | 0.34 | 0.49 | 0.49 | 5.39 | 3.95 | 2 |
| 101 | 324815 | W49563 | 3.60 | 1.71 | 2.11 | 1.59 | 3.05 | 3.40 | 1.60 | 0.92 | 2 |
| 102 | 291097 | N72137 | 7.69 | 3.65 | 2.11 | 0.89 | 1.35 | 3.00 | 1.67 | 3.62 | 2 |
| 103 | 530814 | AA070226 | 47.09 | 22.41 | 2.10 | 3.61 | 0.50 | 0.93 | 2.03 | 3.44 | 2 |
| 104 | 1032405 | AA779457 | 1.81 | 0.86 | 2.10 | 0.76 | 0.67 | 1.22 | 3.46 | 4.38 | 2 |
| 105 | 49303 | H15677 | 0.62 | 0.30 | 2.09 | 3.99 | 0.93 | 0.64 | 1.07 | 3.80 | 2 |
| 106 | 1031701 | AA609566 | 1.73 | 0.83 | 2.07 | 3.33 | 1.92 | 3.02 | 1.06 | 1.03 | 2 |
| 107 | 530185 | AA111969 | 22.57 | 10.94 | 2.06 | 0.84 | 3.63 | 1.80 | 0.44 | 3.61 | 2 |
| 108 | 853998 | AA668897 | 19.09 | 9.27 | 2.06 | 3.08 | 2.55 | 0.49 | 0.84 | 3.34 | 2 |
| 109 | 378813 | AA683520 | 7.78 | 3.81 | 2.04 | 1.09 | 3.29 | 0.41 | 2.36 | 3.05 | 2 |
| 110 | 810899 | AA459292 | 0.02 | 0.01 | 2.01 | 0.56 | 3.70 | 1.13 | 0.40 | 4.26 | 2 |
| 111 | 297392 | N80129 | 50.71 | 25.44 | 1.99 | 0.27 | 0.58 | 0.52 | 4.51 | 4.08 | 2 |
| 112 | 471682 | AA035558 | 4.12 | 2.07 | 1.99 | 0.76 | 1.69 | 0.98 | 3.19 | 3.30 | 2 |
| 113 | 27098 | R36989 | 2.20 | 1.11 | 1.98 | 1.43 | 1.05 | 1.23 | 3.09 | 3.07 | 2 |
| 114 | 810873 | AA459197 | 2.96 | 1.50 | 1.97 | 3.13 | 1.99 | 0.68 | 0.93 | 3.11 | 2 |
| 115 | 485854 | AA040424 | 1.67 | 0.85 | 1.96 | 1.39 | 0.93 | 0.93 | 3.41 | 3.16 | 2 |
| 116 | 293292 | N64706 | 2.97 | 1.51 | 1.96 | 1.40 | 0.75 | 0.78 | 3.03 | 3.85 | 2 |
| 117 | 289337 | N92646 | 4.29 | 2.19 | 1.96 | 0.92 | 3.84 | 0.37 | 0.54 | 4.12 | 2 |
| 118 | 80948 | T70057 | 15.71 | 8.31 | 1.89 | 1.25 | 3.02 | 0.38 | 0.58 | 4.24 | 2 |
| 119 | 838500 | AA457528 | 1.44 | 0.76 | 1.89 | 0.74 | 0.90 | 0.85 | 3.49 | 3.47 | 2 |
| 120 | 343987 | W70234 | 28.35 | 15.04 | 1.89 | 0.91 | 3.19 | 1.87 | 0.41 | 3.05 | 2 |
| 121 | 258300 | N30680 | 25.74 | 13.84 | 1.86 | 0.55 | 3.33 | 4.05 | 0.75 | 0.61 | 2 |
| 122 | 824340 | AA489666 | 5.85 | 3.24 | 1.80 | 0.42 | 0.66 | 0.52 | 3.65 | 3.76 | 2 |
| 123 | 884606 | AA630006 | 37.58 | 21.21 | 1.77 | 0.69 | 1.44 | 0.21 | 3.37 | 3.15 | 2 |
| 124 | 132702 | R27004 | 48.33 | 27.34 | 1.77 | 0.26 | 0.56 | 3.72 | 3.19 | 1.11 | 2 |
| 125 | 767449 | AA418000 | 5.72 | 3.24 | 1.76 | 0.57 | 3.19 | 3.89 | 0.56 | 0.61 | 2 |
| 126 | 35804 | R46000 | 1.59 | 0.94 | 1.69 | 0.36 | 3.18 | 3.81 | 0.63 | 0.49 | 2 |
| 127 | 1031113 | AA609914 | 0.03 | 0.02 | 1.66 | 0.00 | 0.53 | 3.25 | 4.04 | 0.48 | 2 |

TABLE 2-9
Good vs Poor Clinical Outcome:
Genes upregulated 5 fold or more In at least 2
tumor samples.

| | | Gen Bank | | | | Fold | Fold | Fold | Fold | Fold | Fold | # OF |
|---|---|---|---|---|---|---|---|---|---|---|---|---|
| | IMAGE | Accession | Tissue | Ave- | Ave- | (AveGood/ | (mpm27/ | (mpm28/ | (mpm29/ | (mpm30/ | (mpm35/ | Tumor |
| Order | Clone ID | Number | Distribution | Good | Poor | AvePoor | AvePoor | AvePoor | AvePoor | AvePoor | AvePoor | Samples |
| 1 | 281039 | N47717 | bone | 36.67 | 4.42 | 8.29 | 22.87 | 1.40 | 0.27 | 9.85 | 7.08 | 3 |
| 2 | 282089 | N48259 | prostate | 27.13 | 3.45 | 7.87 | 22.41 | 0.41 | 0.67 | 7.05 | 8.80 | 3 |
| 3 | 155072 | R71393 | — | 3.32 | 0.45 | 7.44 | 8.50 | 1.97 | 1.30 | 9.52 | 15.90 | 3 |
| 4 | 208210 | H65288 | testis | 2.70 | 0.42 | 6.48 | 14.00 | 1.98 | 0.98 | 5.30 | 10.17 | 3 |
| 5 | 770088 | AA430545 | brain | 28.72 | 4.82 | 5.78 | 12.05 | 0.67 | 1.90 | 8.30 | 5.96 | 3 |
| 6 | 299879 | W05828 | bone | 2.55 | 0.50 | 5.07 | 7.07 | 6.80 | 3.51 | 0.88 | 7.30 | 3 |
| 7 | 787130 | AA424535 | kidney | 4.28 | 0.37 | 11.65 | 1.60 | 25.69 | 28.44 | 1.49 | 1.02 | 2 |
| 8 | 307337 | N95228 | spleen | 10.81 | 0.96 | 11.29 | 1.65 | 7.47 | 44.45 | 1.65 | 1.22 | 2 |
| 9 | 471841 | AA034945 | brain | 11.03 | 1.08 | 10.17 | 21.19 | 2.20 | 4.22 | 2.10 | 21.15 | 2 |
| 10 | 712950 | AA282273 | t-ceIl | 38.22 | 6.94 | 5.50 | 8.84 | 1.04 | 0.35 | 4.37 | 12.92 | 2 |
| 11 | 41869 | R66438 | adrenal | 1.41 | 0.28 | 5.03 | 1.46 | 5.93 | 15.74 | 1.30 | 0.71 | 2 |
| 12 | 81331 | T60111 | | 4.13 | 0.82 | 6.03 | 11.18 | 1.30 | 0.80 | 8.59 | 3.28 | 2 |
| 13 | 287728 | N59158 | — | 3.49 | 0.71 | 4.90 | 14.39 | 0.35 | 0.46 | 3.86 | 5.42 | 2 |
| 14 | 301735 | N90882 | esophagus | 4.10 | 0.90 | 4.56 | 9.05 | 2.75 | 2.93 | 0.99 | 7.09 | 2 |
| 15 | 811162 | AA488471 | thyroid | 28.87 | 6.85 | 4.21 | 0.70 | 3.61 | 6.33 | 4.17 | 6.26 | 2 |
| 16 | 382278 | AA001219 | lymph | 5.53 | 1.41 | 3.91 | 2.64 | 2.34 | 0.78 | 8.56 | 7.23 | 2 |
| 17 | 1409509 | AA888929 | bone | 2.84 | 0.75 | 3.78 | 0.36 | 5.86 | 11.75 | 0.59 | 0.31 | 2 |
| 18 | 768953 | AA424920 | — | 3.43 | 0.94 | 3.66 | 0.96 | 5.41 | 10.03 | 0.93 | 0.97 | 2 |
| 19 | 357628 | W94121 | — | 1.80 | 0.49 | 3.65 | 5.82 | 0.55 | 0.68 | 2.00 | 9.41 | 2 |
| 20 | 195385 | R88904 | — | 1.81 | 0.50 | 3.62 | 6.03 | 1.45 | 0.88 | 3.51 | 6.23 | 2 |
| 21 | 145740 | R77948 | prostate | 30.23 | 8.55 | 3.53 | 1.47 | 0.52 | 0.73 | 5.51 | 9.44 | 2 |
| 22 | 782335 | AA432270 | endothelial | 4.04 | 1.30 | 3.12 | 6.21 | 5.84 | 1.80 | 0.66 | 1.08 | 2 |

TABLE 2-10
Prostate Cancer Cell Lines vs. Normal Prostastic Epithelial
Cell Line: Genes upregulated fold or higher in 3 of 3, 5 fold or higher In
at least 2 of 3 or 10 fold or higher In at least 1 of 3 cancer cell lines.

| | | Gen Bank | | Ave Cancer | Fold | Fold | Fold | Fold | |
|---|---|---|---|---|---|---|---|---|---|
| | IMAGE | Accession | | Cell Lines | (AveCell | (MPM2/ | (MPM271/ | (MPM272/ | # of Cancer |
| Order | Clone ID | Number | MPM1(PrEC) | Values | line/MPM1) | MPM1) | MPM1) | MPM1) | Cell Lines |
| 1 | 757165 | AA443950 | 0.01 | 5.71 | 591.67 | 7.33 | 66.37 | 1701.31 | 3 |
| 2 | 510631 | AA099431 | 0.01 | 3.19 | 319.36 | 4.57 | 944.19 | 9.33 | 3 |
| 3 | 592707 | AA160606 | 0.01 | 1.66 | 234.30 | 8.10 | 680.66 | 14.13 | 3 |
| 4 | 220851 | H95633 | 0.01 | 1.28 | 196.56 | 8.10 | 570.75 | 10.82 | 3 |
| 5 | 782804 | AA448186 | 0.01 | 1.81 | 180.70 | 8.88 | 526.08 | 7.15 | 3 |
| 6 | 838831 | AA481770 | 0.02 | 2.58 | 117.17 | 3.60 | 344.82 | 3.10 | 3 |
| 7 | 1343732 | AA725564 | 0.01 | 1.12 | 112.48 | 13.45 | 301.13 | 22.85 | 3 |
| 8 | 768226 | AA424900 | 0.01 | 1.04 | 103.67 | 3.09 | 302.09 | 5.84 | 3 |
| 9 | 731048 | AA421275 | 0.01 | 0.99 | 99.19 | 3.74 | 290.43 | 3.41 | 3 |
| 10 | 366039 | AA071503 | 0.03 | 2.66 | 86.03 | 5.01 | 249.63 | 3.45 | 3 |
| 11 | 795627 | AA459917 | 0.01 | 0.51 | 85.17 | 3.09 | 239.88 | 12.54 | 3 |
| 12 | 1161830 | AA876021 | 0.01 | 0.71 | 71.26 | 3.19 | 203.18 | 7.41 | 3 |
| 13 | 731371 | AA421047 | 0.03 | 2.20 | 71.17 | 3.28 | 205.85 | 4.38 | 3 |
| 14 | 784296 | AA447079 | 0.01 | 0.66 | 65.30 | 45.84 | 106.60 | 43.45 | 3 |
| 15 | 796569 | AA460463 | 0.01 | 0.35 | 63.57 | 16.10 | 138.91 | 35.70 | 3 |
| 16 | 346942 | W94289 | 0.01 | 0.61 | 61.20 | 3.73 | 168.10 | 11.76 | 3 |
| 17 | 795858 | AA461522 | 0.03 | 1.60 | 60.34 | 6.29 | 170.43 | 4.30 | 3 |
| 18 | 131452 | R23270 | 0.45 | 26.93 | 59.79 | 104.24 | 4.69 | 70.45 | 3 |
| 19 | 1558655 | AA976581 | 0.08 | 4.36 | 54.32 | 97.08 | 17.92 | 47.97 | 3 |
| 20 | 743016 | AA406036 | 0.01 | 0.50 | 50.47 | 15.70 | 123.80 | 11.93 | 3 |
| 21 | 39442 | R51617 | 0.10 | 5.02 | 49.93 | 99.52 | 44.77 | 5.49 | 3 |
| 22 | 33076 | R44048 | 0.80 | 38.21 | 45.35 | 56.36 | 29.19 | 50.51 | 3 |
| 23 | 626390 | AA189106 | 0.07 | 3.16 | 45.16 | 91.62 | 20.97 | 22.90 | 3 |
| 24 | 128083 | R09747 | 0.01 | 0.44 | 44.34 | 25.03 | 101.07 | 6.93 | 3 |
| 25 | 1389018 | AA855158 | 0.02 | 0.81 | 43.48 | 3.54 | 120.65 | 6.23 | 3 |
| 26 | 28498 | R37395 | 0.01 | 0.43 | 43.37 | 5.62 | 121.33 | 3.16 | 3 |
| 27 | 757368 | AA437126 | 0.01 | 0.42 | 41.72 | 4.59 | 109.53 | 11.05 | 3 |
| 28 | 813633 | AA447740 | 0.04 | 1.68 | 41.51 | 3.55 | 116.08 | 4.92 | 3 |
| 29 | 1367900 | AA810225 | 0.01 | 0.41 | 41.11 | 4.81 | 99.20 | 19.33 | 3 |
| 30 | 366209 | AA062985 | 0.01 | 0.38 | 38.18 | 3.08 | 106.34 | 5.11 | 3 |
| 31 | 812170 | AA456035 | 0.01 | 0.30 | 37.18 | 16.25 | 80.79 | 14.51 | 3 |
| 32 | 50288 | H17888 | 0.01 | 0.32 | 36.79 | 12.25 | 87.45 | 10.68 | 3 |
| 33 | 243181 | H94482 | 0.01 | 0.36 | 35.62 | 5.91 | 88.28 | 12.68 | 3 |
| 34 | 256911 | N30098 | 0.01 | 0.25 | 34.44 | 20.19 | 73.72 | 9.41 | 3 |
| 35 | 1030613 | AA608832 | 0.01 | 0.33 | 33.18 | 5.81 | 82.72 | 11.01 | 3 |
| 36 | 487861 | AA045436 | 0.02 | 0.59 | 32.57 | 29.44 | 39.44 | 28.82 | 3 |
| 37 | 270626 | N33331 | 0.01 | 0.32 | 31.52 | 8.48 | 9.21 | 76.87 | 3 |
| 38 | 291057 | N72115 | 0.23 | 6.85 | 29.19 | 24.48 | 38.30 | 24.80 | 3 |
| 39 | 1455835 | AA863292 | 0.01 | 0.40 | 29.16 | 9.10 | 30.24 | 48.14 | 3 |
| 40 | 108378 | T77729 | 0.01 | 0.41 | 29.13 | 7.73 | 36.47 | 43.19 | 3 |
| 41 | 1391682 | AA789328 | 0.02 | 0.44 | 28.83 | 5.99 | 65.78 | 14.72 | 3 |
| 42 | 263014 | H99813 | 0.63 | 17.34 | 27.31 | 19.07 | 21.59 | 41.29 | 3 |
| 43 | 303180 | N92764 | 0.01 | 0.27 | 27.21 | 13.82 | 36.04 | 31.77 | 3 |
| 44 | 42415 | R60981 | 0.12 | 3.11 | 26.71 | 9.46 | 65.35 | 5.30 | 3 |
| 45 | 768056 | AA418903 | 0.01 | 0.16 | 26.24 | 7.36 | 35.03 | 36.33 | 3 |
| 46 | 46896 | H09818 | 0.05 | 1.21 | 25.96 | 33.86 | 30.16 | 13.87 | 3 |
| 47 | 151184 | H02294 | 0.48 | 12.53 | 25.93 | 38.89 | 13.26 | 25.64 | 3 |
| 48 | 38344 | R49555 | 0.01 | 0.24 | 25.84 | 5.18 | 58.23 | 14.11 | 3 |
| 49 | 787856 | AA452139 | 0.01 | 0.29 | 25.59 | 4.23 | 62.71 | 9.84 | 3 |
| 50 | 41595 | R59556 | 0.01 | 0.17 | 25.59 | 12.08 | 52.20 | 12.48 | 3 |
| 51 | 430255 | AA010383 | 0.01 | 0.25 | 25.24 | 4.46 | 54.64 | 16.63 | 3 |
| 52 | 687972 | AA236986 | 0.01 | 0.17 | 25.09 | 3.61 | 60.05 | 11.62 | 3 |
| 53 | 430092 | AA009840 | 0.40 | 10.00 | 24.80 | 34.18 | 13.51 | 26.60 | 3 |
| 54 | 1049033 | AA778675 | 0.38 | 9.30 | 24.51 | 46.38 | 16.26 | 10.88 | 3 |
| 55 | 884790 | AA629838 | 0.01 | 0.24 | 24.29 | 30.26 | 10.09 | 32.50 | 3 |
| 58 | 23345 | R39191 | 0.01 | 0.16 | 23.97 | 44.37 | 17.55 | 9.98 | 3 |
| 57 | 29585 | R42112 | 0.04 | 0.84 | 23.75 | 8.09 | 59.71 | 3.46 | 3 |
| 58 | 284714 | N63057 | 0.01 | 0.16 | 23.01 | 8.11 | 34.50 | 26.43 | 3 |
| 59 | 855910 | AA630328 | 0.01 | 0.16 | 22.37 | 4.98 | 30.37 | 31.76 | 3 |
| 60 | 34204 | R44936 | 0.17 | 3.74 | 22.30 | 3.82 | 51.95 | 11.14 | 3 |
| 61 | 365149 | AA025142 | 0.16 | 3.52 | 22.24 | 38.20 | 16.95 | 11.55 | 3 |
| 62 | 79000 | T61938 | 1.90 | 40.77 | 21.44 | 37.52 | 23.72 | 3.08 | 3 |
| 63 | 43065 | R61866 | 0.05 | 0.97 | 21.43 | 4.47 | 56.01 | 3.81 | 3 |
| 64 | 259884 | N32904 | 0.91 | 19.16 | 20.97 | 4.40 | 10.88 | 47.62 | 3 |
| 65 | 418094 | W90036 | 0.01 | 0.21 | 20.90 | 5.07 | 7.89 | 49.72 | 3 |
| 66 | 51581 | H22824 | 0.01 | 0.25 | 20.42 | 18.18 | 18.45 | 24.62 | 3 |
| 67 | 270975 | N32542 | 0.01 | 0.20 | 20.40 | 5.36 | 33.42 | 22.42 | 3 |
| 68 | 51916 | H22563 | 0.12 | 2.46 | 20.27 | 20.94 | 30.57 | 9.30 | 3 |
| 69 | 40010 | R54036 | 0.01 | 0.19 | 19.50 | 7.04 | 11.18 | 40.27 | 3 |
| 70 | 39722 | R54492 | 0.01 | 0.17 | 18.85 | 3.39 | 10.12 | 43.06 | 3 |
| 71 | 1467161 | AA883187 | 0.02 | 0.31 | 18.24 | 10.20 | 24.35 | 20.16 | 3 |
| 72 | 489076 | AA057195 | 0.02 | 0.35 | 18.01 | 4.49 | 38.83 | 10.71 | 3 |
| 73 | 488431 | AA047441 | 0.17 | 2.95 | 17.83 | 10.45 | 28.90 | 14.13 | 3 |
| 74 | 435099 | AA701315 | 0.01 | 0.21 | 17.36 | 7.52 | 33.82 | 10.73 | 3 |
| 75 | 67440 | T49355 | 1.96 | 33.78 | 17.25 | 25.95 | 8.91 | 16.88 | 3 |
| 76 | 1031568 | AA609304 | 0.05 | 0.86 | 17.19 | 4.26 | 42.11 | 5.21 | 3 |
| 77 | 280155 | N47012 | 0.04 | 0.63 | 17.15 | 5.64 | 11.78 | 34.04 | 3 |
| 78 | 627521 | AA192784 | 0.04 | 0.69 | 16.92 | 4.19 | 42.48 | 4.08 | 3 |
| 79 | 454083 | AA676998 | 0.01 | 0.17 | 16.89 | 8.16 | 10.67 | 31.84 | 3 |
| 80 | 347586 | W81432 | 0.01 | 0.17 | 16.56 | 7.84 | 8.92 | 32.94 | 3 |
| 81 | 269606 | N26769 | 0.01 | 0.21 | 16.54 | 6.72 | 36.98 | 5.91 | 3 |
| 82 | 358990 | W92263 | 0.01 | 0.16 | 16.35 | 10.47 | 5.09 | 33.48 | 3 |
| 83 | 235164 | H79466 | 0.01 | 0.09 | 16.34 | 9.38 | 30.25 | 9.40 | 3 |
| 84 | 357373 | W93717 | 0.70 | 11.31 | 16.17 | 5.81 | 15.89 | 26.82 | 3 |
| 85 | 238689 | H81554 | 0.07 | 1.17 | 15.96 | 3.05 | 41.27 | 3.54 | 3 |
| 88 | 82869 | T69270 | 0.02 | 0.32 | 15.94 | 21.48 | 14.07 | 12.25 | 3 |
| 87 | 753411 | AA410437 | 0.03 | 0.40 | 15.85 | 3.94 | 36.93 | 6.69 | 3 |
| 88 | 767315 | AA418545 | 0.02 | 0.29 | 15.63 | 6.61 | 33.79 | 6.49 | 3 |
| 89 | 487766 | AA045175 | 0.02 | 0.30 | 15.57 | 4.69 | 22.78 | 19.22 | 3 |
| 90 | 742698 | AA401378 | 0.05 | 0.71 | 15.34 | 3.35 | 39.06 | 3.61 | 3 |
| 91 | 447416 | AA702335 | 0.25 | 3.80 | 15.00 | 33.97 | 5.66 | 5.36 | 3 |
| 92 | 250069 | H97140 | 0.02 | 0.35 | 14.92 | 5.14 | 24.61 | 15.00 | 3 |
| 93 | 743030 | AA406061 | 0.05 | 0.70 | 14.84 | 3.83 | 37.53 | 3.17 | 3 |
| 94 | 28298 | R40434 | 0.02 | 0.35 | 14.61 | 5.11 | 16.48 | 22.27 | 3 |
| 95 | 757389 | AA437142 | 0.01 | 0.14 | 14.54 | 5.64 | 32.86 | 5.13 | 3 |
| 96 | 785293 | AA476543 | 0.11 | 1.64 | 14.41 | 24.69 | 3.02 | 15.54 | 3 |
| 97 | 1461609 | AA883800 | 0.25 | 3.51 | 14.24 | 10.07 | 4.19 | 28.45 | 3 |
| 98 | 25915 | R39951 | 0.01 | 0.14 | 14.20 | 9.65 | 6.51 | 26.44 | 3 |
| 99 | 1031446 | AA609189 | 0.04 | 0.50 | 14.14 | 5.44 | 31.74 | 5.25 | 3 |
| 100 | 32109 | R42780 | 0.09 | 1.25 | 14.14 | 31.21 | 5.86 | 5.35 | 3 |
| 101 | 856289 | AA774665 | 1.24 | 17.58 | 14.13 | 5.00 | 9.24 | 28.16 | 3 |
| 102 | 30850 | R42600 | 0.03 | 0.39 | 13.78 | 6.39 | 29.82 | 5.12 | 3 |
| 103 | 41406 | R56149 | 0.01 | 0.13 | 13.76 | 17.85 | 18.97 | 4.48 | 3 |
| 104 | 1035796 | AA628867 | 1.14 | 15.63 | 13.71 | 10.64 | 9.76 | 20.74 | 3 |
| 105 | 898286 | AA598974 | 1.21 | 16.52 | 13.65 | 13.47 | 11.86 | 15.62 | 3 |
| 106 | 502003 | AA128617 | 0.01 | 0.17 | 13.58 | 7.33 | 15.99 | 17.41 | 3 |
| 107 | 359269 | AA016225 | 0.01 | 0.13 | 13.43 | 9.24 | 8.92 | 22.12 | 3 |
| 108 | 415089 | W93379 | 0.65 | 8.63 | 13.24 | 8.23 | 12.04 | 19.45 | 3 |
| 109 | 1466409 | AA885478 | 0.14 | 1.91 | 13.23 | 22.16 | 9.42 | 8.12 | 3 |
| 110 | 824074 | AA491227 | 0.02 | 0.26 | 13.22 | 3.39 | 18.08 | 18.18 | 3 |
| 111 | 51103 | H19217 | 0.02 | 0.23 | 13.14 | 14.72 | 17.85 | 6.86 | 3 |
| 112 | 1467166 | AA883181 | 0.08 | 1.05 | 13.14 | 3.08 | 29.16 | 7.18 | 3 |
| 113 | 244050 | N34042 | 0.01 | 0.17 | 12.88 | 5.65 | 21.72 | 11.26 | 3 |
| 114 | 1055121 | AA621355 | 0.02 | 0.25 | 12.67 | 6.31 | 28.51 | 3.21 | 3 |
| 115 | 897807 | AA598531 | 0.29 | 3.56 | 12.37 | 21.95 | 7.81 | 7.37 | 3 |
| 116 | 845441 | AA644550 | 0.01 | 0.12 | 12.30 | 5.26 | 7.58 | 24.06 | 3 |
| 117 | 686172 | AA262211 | 1.18 | 14.39 | 12.18 | 5.65 | 13.80 | 17.09 | 3 |
| 118 | 1049079 | AA778717 | 0.03 | 0.31 | 12.10 | 3.28 | 15.47 | 17.55 | 3 |
| 119 | 785368 | AA476576 | 1.38 | 16.58 | 12.06 | 8.77 | 4.23 | 23.17 | 3 |
| 120 | 451649 | AA706901 | 1.40 | 16.82 | 11.98 | 4.34 | 18.70 | 12.88 | 3 |
| 121 | 48461 | H09940 | 0.33 | 3.89 | 11.80 | 3.09 | 13.18 | 19.11 | 3 |
| 122 | 1030798 | AA609002 | 0.03 | 0.31 | 11.73 | 10.15 | 8.33 | 16.72 | 3 |
| 123 | 33839 | R44816 | 0.96 | 11.24 | 11.67 | 3.60 | 19.31 | 12.09 | 3 |
| 124 | 357138 | W93523 | 0.02 | 0.20 | 11.63 | 10.87 | 20.61 | 3.40 | 3 |
| 125 | 366971 | AA026682 | 2.38 | 27.64 | 11.61 | 3.65 | 10.18 | 20.99 | 3 |
| 126 | 609228 | AA179161 | 0.01 | 0.11 | 11.46 | 4.58 | 5.70 | 24.11 | 3 |
| 127 | 210820 | H67712 | 0.04 | 0.45 | 11.30 | 5.06 | 7.88 | 20.94 | 3 |
| 128 | 795746 | AA460299 | 0.52 | 5.68 | 10.87 | 17.77 | 9.89 | 4.94 | 3 |
| 129 | 795840 | AA461513 | 0.01 | 0.06 | 10.81 | 4.64 | 15.78 | 12.02 | 3 |
| 130 | 788209 | AA453433 | 0.01 | 0.09 | 10.70 | 11.07 | 8.10 | 12.92 | 3 |
| 131 | 767403 | AA417920 | 0.56 | 5.93 | 10.66 | 4.43 | 22.31 | 5.22 | 3 |
| 132 | 204483 | H58234 | 1.27 | 13.51 | 10.64 | 11.48 | 13.18 | 7.26 | 3 |
| 133 | 814769 | AA454949 | 1.16 | 12.13 | 10.45 | 21.90 | 4.33 | 5.14 | 3 |
| 134 | 430928 | AA678295 | 0.74 | 7.64 | 10.34 | 20.21 | 5.77 | 5.03 | 3 |
| 135 | 745019 | AA626028 | 0.01 | 0.10 | 10.24 | 3.65 | 22.27 | 4.82 | 3 |
| 136 | 725308 | AA291556 | 0.02 | 0.18 | 10.15 | 3.39 | 4.07 | 22.97 | 3 |
| 137 | 898073 | AA598787 | 0.04 | 0.37 | 10.00 | 3.74 | 20.48 | 5.78 | 3 |
| 138 | 745490 | AA625979 | 0.11 | 1.05 | 9.95 | 12.71 | 3.07 | 14.06 | 3 |
| 139 | 34134 | R44734 | 0.51 | 5.07 | 9.92 | 21.35 | 3.47 | 4.93 | 3 |
| 140 | 391987 | AI003636 | 0.06 | 0.57 | 9.79 | 3.40 | 9.94 | 16.03 | 3 |
| 141 | 376452 | AA041197 | 0.03 | 0.32 | 9.78 | 5.69 | 8.58 | 15.10 | 3 |
| 142 | 431242 | AA682533 | 1.26 | 12.26 | 9.74 | 3.45 | 18.90 | 6.87 | 3 |
| 143 | 590500 | AA157261 | 0.02 | 0.17 | 9.64 | 4.99 | 9.81 | 14.13 | 3 |
| 144 | 590727 | AA156342 | 0.01 | 0.10 | 9.62 | 3.83 | 6.58 | 18.44 | 3 |
| 145 | 433567 | AA701652 | 0.02 | 0.16 | 9.57 | 11.04 | 10.28 | 7.37 | 3 |
| 146 | 273546 | N33274 | 3.29 | 31.33 | 9.53 | 4.57 | 20.10 | 3.91 | 3 |
| 147 | 280375 | N47113 | 1.12 | 10.61 | 9.52 | 11.98 | 10.79 | 5.79 | 3 |
| 148 | 303099 | N90779 | 0.15 | 1.40 | 9.48 | 5.53 | 11.42 | 11.50 | 3 |
| 149 | 448059 | AA702684 | 0.22 | 2.04 | 9.46 | 6.47 | 14.40 | 7.50 | 3 |
| 150 | 269300 | N24042 | 1.12 | 10.53 | 9.44 | 5.48 | 13.22 | 9.62 | 3 |
| 151 | 430973 | AA678348 | 0.76 | 7.14 | 9.41 | 3.48 | 5.97 | 18.79 | 3 |
| 152 | 645565 | AA204830 | 0.62 | 5.85 | 9.41 | 4.56 | 7.97 | 15.70 | 3 |
| 153 | 346552 | W74377 | 0.02 | 0.19 | 9.32 | 3.79 | 15.51 | 8.67 | 3 |
| 154 | 489213 | AA045665 | 0.37 | 3.43 | 9.32 | 9.36 | 11.24 | 7.35 | 3 |
| 155 | 43733 | H04789 | 0.11 | 1.03 | 9.24 | 10.06 | 12.44 | 5.24 | 3 |
| 156 | 461488 | AA705047 | 0.10 | 0.97 | 9.22 | 12.49 | 10.86 | 4.33 | 3 |
| 157 | 781047 | AA446462 | 1.08 | 9.89 | 9.17 | 4.58 | 11.63 | 11.30 | 3 |
| 158 | 234617 | H77727 | 0.07 | 0.60 | 9.16 | 12.99 | 8.66 | 5.84 | 3 |
| 159 | 46694 | H10192 | 0.35 | 3.21 | 9.15 | 8.17 | 14.83 | 4.46 | 3 |
| 160 | 177884 | H46176 | 0.16 | 1.44 | 9.15 | 5.38 | 8.45 | 13.62 | 3 |
| 161 | 897956 | AA598817 | 0.15 | 1.40 | 9.13 | 13.21 | 8.47 | 5.71 | 3 |
| 162 | 141726 | R69584 | 0.03 | 0.26 | 9.09 | 3.46 | 10.73 | 13.08 | 3 |
| 163 | 324901 | W49672 | 0.01 | 0.11 | 8.92 | 7.38 | 12.47 | 6.90 | 3 |
| 164 | 78217 | T50699 | 0.01 | 0.05 | 8.82 | 9.14 | 7.21 | 10.11 | 3 |
| 165 | 624627 | AA187351 | 0.56 | 4.90 | 8.72 | 5.30 | 9.17 | 11.70 | 3 |
| 166 | 491184 | AA137072 | 1.49 | 12.90 | 8.67 | 6.69 | 13.78 | 5.53 | 3 |
| 167 | 242952 | H95638 | 5.65 | 48.92 | 8.66 | 3.86 | 6.65 | 15.48 | 3 |
| 168 | 826273 | AA520999 | 0.45 | 3.90 | 8.62 | 5.02 | 10.99 | 9.84 | 3 |
| 169 | 71977 | T52311 | 0.02 | 0.14 | 8.51 | 7.42 | 14.21 | 3.90 | 3 |
| 170 | 840150 | AA485265 | 0.02 | 0.19 | 8.35 | 4.95 | 13.73 | 6.37 | 3 |
| 171 | 809557 | AA455786 | 0.53 | 4.39 | 8.32 | 3.89 | 6.65 | 14.40 | 3 |
| 172 | 42906 | R60019 | 0.19 | 1.57 | 8.31 | 9.78 | 8.33 | 6.83 | 3 |
| 173 | 344134 | W73790 | 0.01 | 0.08 | 8.30 | 4.16 | 16.37 | 4.37 | 3 |
| 174 | 773383 | AA425755 | 0.94 | 7.83 | 8.30 | 5.27 | 11.10 | 8.51 | 3 |
| 175 | 358599 | W96216 | 0.03 | 0.25 | 8.13 | 3.04 | 13.64 | 7.71 | 3 |
| 176 | 30148 | R41329 | 2.63 | 21.34 | 8.13 | 4.35 | 5.10 | 14.93 | 3 |
| 177 | 203275 | H54752 | 1.29 | 10.44 | 8.09 | 5.06 | 3.98 | 15.23 | 3 |
| 178 | 80399 | T65902 | 7.67 | 61.74 | 8.05 | 4.69 | 4.12 | 15.35 | 3 |
| 179 | 1292182 | AA705825 | 0.03 | 0.28 | 8.02 | 3.42 | 7.53 | 13.11 | 3 |
| 180 | 52741 | H29521 | 0.70 | 5.63 | 8.01 | 3.96 | 15.49 | 4.58 | 3 |
| 181 | 813271 | AA455938 | 0.07 | 0.54 | 7.87 | 11.86 | 6.62 | 5.12 | 3 |
| 182 | 190325 | H29897 | 0.37 | 2.89 | 7.77 | 4.86 | 11.97 | 6.49 | 3 |
| 183 | 745393 | AA625764 | 0.08 | 0.60 | 7.77 | 3.94 | 9.24 | 10.12 | 3 |
| 184 | 782259 | AA431741 | 0.75 | 5.81 | 7.75 | 11.54 | 7.24 | 4.46 | 3 |
| 185 | 1505919 | AA906257 | 0.05 | 0.35 | 7.72 | 4.23 | 8.93 | 10.00 | 3 |
| 186 | 121251 | T98718 | 3.19 | 24.59 | 7.70 | 7.55 | 4.17 | 11.39 | 3 |
| 187 | 73596 | T55607 | 1.74 | 13.42 | 7.70 | 4.60 | 7.66 | 10.84 | 3 |
| 188 | 322461 | W15305 | 0.22 | 1.68 | 7.70 | 6.23 | 3.58 | 13.28 | 3 |
| 189 | 882510 | AA676460 | 34.04 | 261.61 | 7.68 | 3.35 | 8.00 | 11.71 | 3 |
| 190 | 200402 | R9694l | 0.75 | 5.69 | 7.61 | 8.56 | 5.22 | 9.05 | 3 |
| 191 | 950690 | AA608568 | 0.73 | 5.53 | 7.61 | 7.77 | 7.09 | 7.97 | 3 |
| 192 | 260035 | N30372 | 0.02 | 0.15 | 7.55 | 6.04 | 5.10 | 11.50 | 3 |
| 193 | 530820 | AA070331 | 0.01 | 0.04 | 7.55 | 10.53 | 4.96 | 7.15 | 3 |
| 194 | 770879 | AA434403 | 0.75 | 5.64 | 7.53 | 3.47 | 10.88 | 8.23 | 3 |
| 195 | 83666 | T61122 | 0.03 | 0.20 | 7.44 | 12.84 | 4.41 | 5.28 | 3 |
| 196 | 435076 | AA701455 | 2.95 | 21.88 | 7.41 | 10.35 | 6.94 | 4.93 | 3 |
| 197 | 309288 | N93924 | 3.23 | 23.90 | 7.39 | 5.03 | 4.68 | 12.46 | 3 |
| 198 | 843163 | AA488367 | 7.80 | 57.27 | 7.34 | 3.00 | 6.84 | 12.18 | 3 |
| 199 | 52704 | H29227 | 0.53 | 3.90 | 7.31 | 7.30 | 6.65 | 7.87 | 3 |
| 200 | 740620 | AA477400 | 0.02 | 0.12 | 7.30 | 6.01 | 6.44 | 9.46 | 3 |
| 201 | 294273 | N70714 | 0.52 | 3.80 | 7.28 | 3.23 | 4.60 | 14.01 | 3 |
| 202 | 50787 | H16833 | 0.06 | 0.41 | 7.24 | 3.25 | 13.99 | 4.48 | 3 |
| 203 | 85670 | T62072 | 0.80 | 5.80 | 7.23 | 4.11 | 7.03 | 10.54 | 3 |
| 204 | 251936 | H97488 | 4.68 | 33.83 | 7.22 | 4.39 | 11.90 | 5.38 | 3 |
| 205 | 796114 | AA460952 | 0.94 | 6.75 | 7.20 | 3.01 | 9.60 | 8.99 | 3 |
| 206 | 853368 | AA663310 | 2.51 | 18.03 | 7.18 | 5.15 | 7.63 | 8.78 | 3 |
| 207 | 809567 | AA456611 | 0.01 | 0.10 | 7.15 | 4.40 | 6.35 | 10.69 | 3 |
| 208 | 283173 | N45236 | 2.09 | 14.78 | 7.08 | 11.72 | 5.87 | 3.65 | 3 |
| 209 | 126650 | R06944 | 0.43 | 3.05 | 7.05 | 5.35 | 7.83 | 7.98 | 3 |
| 210 | 143385 | R74203 | 0.01 | 0.08 | 7.03 | 4.65 | 9.95 | 6.50 | 3 |
| 211 | 1637343 | A1015359 | 14.33 | 100.32 | 7.00 | 4.74 | 7.07 | 9.20 | 3 |
| 212 | 415906 | W86196 | 0.06 | 0.44 | 6.96 | 3.01 | 6.52 | 11.35 | 3 |
| 213 | 40773 | R56046 | 0.06 | 0.39 | 6.93 | 6.40 | 11.26 | 3.14 | 3 |
| 214 | 264502 | N20305 | 0.53 | 3.69 | 6.92 | 6.49 | 4.41 | 9.86 | 3 |
| 215 | 345525 | W72437 | 3.93 | 27.15 | 6.92 | 13.48 | 3.38 | 3.89 | 3 |
| 216 | 814225 | AA465238 | 1.08 | 7.46 | 6.92 | 4.76 | 10.31 | 5.69 | 3 |
| 217 | 433576 | AA701645 | 0.10 | 0.72 | 6.85 | 10.00 | 5.36 | 5.19 | 3 |
| 218 | 246659 | N57722 | 0.37 | 2.56 | 6.83 | 3.47 | 4.92 | 12.11 | 3 |
| 219 | 131316 | R23055 | 1.19 | 8.14 | 6.82 | 3.20 | 5.03 | 12.23 | 3 |
| 220 | 681906 | AA256231 | 0.01 | 0.08 | 6.79 | 4.75 | 10.24 | 5.38 | 3 |
| 221 | 214604 | H71230 | 0.05 | 0.34 | 8.79 | 9.29 | 4.78 | 6.30 | 3 |
| 222 | 46091 | H08595 | 1.32 | 8.93 | 6.79 | 6.37 | 9.34 | 4.64 | 3 |
| 223 | 359119 | AA010065 | 4.09 | 27.70 | 6.77 | 5.57 | 3.78 | 10.97 | 3 |
| 224 | 51083 | H17139 | 0.04 | 0.30 | 6.72 | 7.84 | 4.68 | 7.65 | 3 |
| 225 | 1031885 | AA609723 | 0.54 | 3.61 | 6.72 | 8.02 | 8.01 | 4.14 | 3 |
| 226 | 134719 | R28287 | 0.31 | 2.07 | 6.69 | 9.45 | 5.02 | 5.60 | 3 |
| 227 | 814246 | AA465593 | 9.85 | 65.62 | 6.66 | 3.37 | 9.66 | 6.95 | 3 |
| 228 | 950445 | AA599092 | 2.82 | 18.79 | 6.65 | 7.13 | 4.39 | 8.44 | 3 |
| 229 | 813149 | AA456695 | 0.70 | 4.67 | 6.65 | 7.85 | 4.47 | 7.62 | 3 |
| 230 | 204214 | H59203 | 0.68 | 4.52 | 6.64 | 8.09 | 3.71 | 8.12 | 3 |
| 231 | 1468597 | AA884622 | 0.01 | 0.07 | 6.59 | 7.52 | 7.44 | 4.80 | 3 |
| 232 | 725454 | AA397813 | 5.43 | 35.52 | 6.54 | 3.80 | 3.32 | 12.51 | 3 |
| 233 | 43662 | H05645 | 0.06 | 0.42 | 6.54 | 10.89 | 5.69 | 3.05 | 3 |
| 234 | 51460 | H20847 | 1.12 | 7.31 | 6.53 | 5.27 | 9.69 | 4.83 | 3 |
| 235 | 281733 | N48075 | 0.41 | 2.64 | 6.52 | 4.93 | 8.97 | 5.67 | 3 |
| 236 | 50383 | H17869 | 3.43 | 22.29 | 6.50 | 4.19 | 4.30 | 11.00 | 3 |
| 237 | 160233 | H21943 | 1.30 | 8.44 | 6.48 | 4.26 | 5.16 | 10.01 | 3 |
| 238 | 487297 | AA045508 | 1.13 | 7.26 | 6.45 | 3.07 | 8.32 | 7.97 | 3 |
| 239 | 430007 | AA034115 | 0.26 | 1.70 | 6.43 | 4.03 | 5.78 | 9.48 | 3 |
| 240 | 395955 | AA757564 | 0.13 | 0.82 | 6.41 | 5.00 | 4.97 | 9.25 | 3 |
| 241 | 782476 | AA431753 | 0.76 | 4.82 | 6.38 | 8.52 | 6.40 | 4.22 | 3 |
| 242 | 343298 | W68182 | 0.10 | 0.62 | 6.37 | 10.68 | 3.55 | 4.86 | 3 |
| 243 | 151055 | H02231 | 7.49 | 47.65 | 6.36 | 6.67 | 5.78 | 6.63 | 3 |
| 244 | 280907 | N50827 | 0.07 | 0.47 | 6.32 | 3.73 | 12.06 | 3.15 | 3 |
| 245 | 32050 | R41972 | 0.44 | 2.79 | 6.31 | 4.19 | 3.05 | 11.67 | 3 |
| 246 | 782594 | AA447531 | 0.01 | 0.04 | 6.30 | 3.56 | 10.41 | 4.91 | 3 |
| 247 | 292936 | N63744 | 0.72 | 4.53 | 6.29 | 3.26 | 6.93 | 8.69 | 3 |
| 248 | 130004 | R11605 | 0.38 | 2.35 | 6.25 | 3.19 | 5.05 | 10.52 | 3 |
| 249 | 22895 | R38640 | 0.02 | 0.15 | 6.24 | 3.63 | 10.49 | 4.60 | 3 |
| 250 | 249603 | H84871 | 5.09 | 31.76 | 6.24 | 11.55 | 3.15 | 4.01 | 3 |
| 251 | 796694 | AA460685 | 6.55 | 40.75 | 6.23 | 4.27 | 10.33 | 4.07 | 3 |
| 252 | 813586 | AA47662 | 1.97 | 12.17 | 6.19 | 4.19 | 5.59 | 8.79 | 3 |
| 253 | 137890 | R68581 | 0.76 | 4.70 | 6.17 | 4.68 | 3.86 | 9.96 | 3 |
| 254 | 128947 | R10284 | 1.20 | 7.33 | 6.13 | 6.92 | 5.67 | 5.79 | 3 |
| 255 | 50983 | H18017 | 0.02 | 0.14 | 8.12 | 7.62 | 5.90 | 4.83 | 3 |
| 256 | 742115 | AA405800 | 0.57 | 3.47 | 6.11 | 9.63 | 4.21 | 4.48 | 3 |
| 257 | 773073 | AA425302 | 2.73 | 16.66 | 6.10 | 7.47 | 5.73 | 5.08 | 3 |
| 258 | 256680 | H96392 | 0.81 | 4.93 | 6.06 | 3.55 | 6.77 | 7.86 | 3 |
| 259 | 489805 | AA102068 | 0.01 | 0.08 | 6.06 | 6.43 | 4.55 | 7.19 | 3 |
| 260 | 814306 | AA459318 | 4.89 | 29.59 | 6.05 | 6.00 | 6.93 | 5.21 | 3 |
| 261 | 773147 | AA425404 | 1.31 | 7.89 | 6.04 | 4.66 | 8.09 | 5.37 | 3 |
| 262 | 951068 | AA620437 | 0.16 | 0.94 | 6.02 | 9.62 | 3.03 | 5.40 | 3 |
| 263 | 275871 | R93875 | 0.01 | 0.07 | 5.99 | 5.03 | 7.41 | 5.52 | 3 |
| 264 | 271076 | N29918 | 0.54 | 3.18 | 5.90 | 7.03 | 3.15 | 7.53 | 3 |
| 265 | 301104 | N81049 | 0.24 | 1.38 | 5.88 | 9.69 | 3.97 | 3.97 | 3 |
| 266 | 813827 | AA453714 | 0.02 | 0.10 | 5.87 | 3.43 | 4.55 | 9.64 | 3 |
| 267 | 46154 | H09065 | 0.01 | 0.06 | 5.87 | 3.67 | 4.93 | 9.01 | 3 |
| 268 | 489594 | AA099523 | 0.37 | 2.15 | 5.86 | 4.97 | 9.49 | 3.11 | 3 |
| 269 | 357531 | W94009 | 1.37 | 8.00 | 5.86 | 4.55 | 5.43 | 7.60 | 3 |
| 270 | 44255 | H06113 | 2.22 | 12.98 | 5.83 | 3.46 | 4.46 | 9.58 | 3 |
| 271 | 809535 | AA454585 | 7.47 | 43.39 | 5.81 | 3.50 | 5.22 | 8.71 | 3 |
| 272 | 41198 | R58870 | 2.45 | 14.21 | 5.81 | 3.49 | 4.78 | 9.15 | 3 |
| 273 | 287637 | N59136 | 1.87 | 10.85 | 5.80 | 4.53 | 5.30 | 7.58 | 3 |
| 274 | 745360 | AA625662 | 5.55 | 32.21 | 5.80 | 3.92 | 7.27 | 6.22 | 3 |
| 275 | 74738 | T57359 | 2.36 | 13.63 | 5.78 | 3.39 | 5.86 | 8.09 | 3 |
| 276 | 147385 | H01332 | 0.03 | 0.16 | 5.76 | 4.97 | 6.03 | 6.29 | 3 |
| 277 | 884822 | AA669341 | 41.24 | 237.42 | 5.76 | 3.75 | 3.12 | 10.40 | 3 |
| 278 | 795749 | AA460310 | 0.62 | 3.53 | 5.72 | 10.19 | 3.61 | 3.34 | 3 |
| 279 | 504826 | AA150777 | 0.83 | 4.73 | 5.69 | 6.56 | 5.66 | 4.86 | 3 |
| 280 | 147826 | R81486 | 0.92 | 5.22 | 5.68 | 8.14 | 3.99 | 4.93 | 3 |
| 281 | 667598 | AA228130 | 0.90 | 5.08 | 5.66 | 3.80 | 5.70 | 7.68 | 3 |
| 282 | 884799 | AA629849 | 2.81 | 15.89 | 5.65 | 4.60 | 4.10 | 8.24 | 3 |
| 283 | 45882 | H08785 | 0.34 | 1.94 | 5.63 | 5.49 | 5.63 | 5.77 | 3 |
| 284 | 705064 | AA279990 | 1.55 | 8.72 | 5.63 | 3.52 | 4.25 | 9.11 | 3 |
| 285 | 146882 | R80790 | 4.49 | 25.20 | 5.62 | 6.88 | 4.48 | 5.48 | 3 |
| 286 | 399562 | AA733061 | 1.43 | 8.04 | 5.61 | 3.30 | 3.47 | 10.06 | 3 |
| 287 | 220700 | H93424 | 0.96 | 5.38 | 5.61 | 5.49 | 4.88 | 6.45 | 3 |
| 288 | 132708 | R25614 | 0.45 | 2.53 | 5.59 | 9.91 | 3.75 | 3.10 | 3 |
| 289 | 51465 | H23983 | 0.07 | 0.40 | 5.54 | 4.68 | 6.93 | 5.02 | 3 |
| 290 | 85384 | T71965 | 4.40 | 24.34 | 5.54 | 8.15 | 3.92 | 4.54 | 3 |
| 291 | 491615 | AA115559 | 0.93 | 5.08 | 5.49 | 3.62 | 6.29 | 6.56 | 3 |
| 292 | 264146 | N20593 | 0.51 | 2.80 | 5.45 | 3.31 | 9.67 | 3.38 | 3 |
| 293 | 32299 | R42685 | 1.58 | 8.62 | 5.45 | 4.98 | 3.50 | 7.85 | 3 |
| 294 | 361069 | AA017213 | 0.06 | 0.31 | 5.44 | 3.64 | 5.09 | 7.59 | 3 |
| 295 | 898097 | AA598803 | 1.04 | 5.65 | 5.42 | 6.63 | 5.49 | 4.14 | 3 |
| 296 | 459947 | AA779383 | 0.30 | 1.59 | 5.37 | 4.66 | 5.58 | 5.87 | 3 |
| 297 | 789204 | AA450205 | 2.16 | 11.60 | 5.37 | 5.05 | 3.79 | 7.27 | 3 |
| 298 | 431245 | AA682545 | 1.90 | 10.19 | 5.37 | 3.91 | 5.22 | 6.98 | 3 |
| 299 | 460114 | AA676840 | 0.10 | 0.55 | 5.34 | 3.39 | 7.08 | 5.55 | 3 |
| 300 | 357285 | W93882 | 0.18 | 0.94 | 5.33 | 3.66 | 6.07 | 6.26 | 3 |
| 301 | 951125 | AA620556 | 6.73 | 35.80 | 5.32 | 4.34 | 5.23 | 6.39 | 3 |
| 302 | 279752 | N49079 | 1.26 | 6.70 | 5.32 | 3.21 | 3.61 | 9.14 | 3 |
| 303 | 261714 | H98822 | 2.57 | 13.64 | 5.30 | 6.09 | 5.91 | 3.90 | 3 |
| 304 | 26297 | R20628 | 0.02 | 0.10 | 5.24 | 6.79 | 4.32 | 4.61 | 3 |
| 305 | 180841 | R87758 | 0.17 | 0.91 | 5.24 | 8.25 | 3.79 | 3.67 | 3 |
| 306 | 363103 | AA019511 | 0.69 | 3.59 | 5.21 | 7.86 | 4.68 | 3.08 | 3 |
| 307 | 40299 | R52085 | 0.02 | 0.12 | 5.20 | 5.19 | 5.01 | 5.40 | 3 |
| 308 | 840506 | AA485898 | 16.19 | 83.75 | 5.17 | 4.37 | 3.47 | 7.68 | 3 |
| 309 | 490060 | AA136060 | 0.48 | 2.47 | 5.16 | 3.25 | 5.27 | 6.97 | 3 |
| 310 | 447208 | AA703000 | 1.01 | 5.19 | 5.15 | 4.99 | 5.00 | 5.45 | 3 |
| 311 | 278483 | N66132 | 6.20 | 31.82 | 5.13 | 4.34 | 5.12 | 5.93 | 3 |
| 312 | 147147 | R80387 | 0.08 | 0.40 | 5.12 | 6.39 | 4.23 | 4.75 | 3 |
| 313 | 855390 | AA663995 | 0.05 | 0.24 | 5.12 | 3.45 | 3.76 | 8.15 | 3 |
| 314 | 789182 | AA450265 | 6.64 | 34.01 | 5.12 | 7.16 | 3.23 | 4.97 | 3 |
| 315 | 50649 | H18068 | 0.56 | 2.84 | 5.11 | 3.62 | 7.05 | 4.67 | 3 |
| 316 | 1500388 | AA885851 | 0.15 | 0.77 | 5.09 | 4.62 | 5.03 | 5.63 | 3 |
| 317 | 810264 | AA463924 | 2.14 | 10.81 | 5.06 | 4.64 | 6.00 | 4.55 | 3 |
| 318 | 307119 | N93715 | 4.58 | 23.18 | 5.06 | 6.45 | 4.38 | 4.34 | 3 |
| 319 | 782765 | AA448160 | 1.77 | 8.96 | 5.06 | 4.26 | 4.66 | 6.25 | 3 |
| 320 | 505864 | AA683557 | 0.20 | 1.02 | 5.02 | 4.49 | 5.78 | 4.80 | 3 |
| 321 | 825411 | AA504272 | 4.68 | 23.47 | 5.02 | 3.05 | 6.66 | 5.33 | 3 |
| 322 | 564803 | AA129552 | 0.91 | 4.55 | 5.01 | 6.00 | 3.76 | 5.26 | 3 |
| 323 | 877767 | AA626784 | 0.15 | 0.75 | 4.96 | 5.36 | 5.47 | 4.03 | 3 |
| 324 | 277163 | N40939 | 4.90 | 24.26 | 4.95 | 3.28 | 4.74 | 6.82 | 3 |
| 325 | 757206 | AA443969 | 2.34 | 11.53 | 4.92 | 3.32 | 5.83 | 5.61 | 3 |
| 326 | 810974 | AA459632 | 1.37 | 6.70 | 4.90 | 4.46 | 3.84 | 6.41 | 3 |
| 327 | 767994 | AA418918 | 0.54 | 2.66 | 4.87 | 3.38 | 5.34 | 5.90 | 3 |
| 328 | 431895 | AA678135 | 0.32 | 1.54 | 4.83 | 5.12 | 4.57 | 4.80 | 3 |
| 329 | 502296 | AA156599 | 0.19 | 0.90 | 4.81 | 3.32 | 5.49 | 5.64 | 3 |
| 330 | 1292828 | AA776692 | 0.17 | 0.81 | 4.81 | 3.64 | 4.24 | 6.54 | 3 |
| 331 | 951142 | AA620553 | 3.26 | 15.65 | 4.81 | 5.78 | 4.27 | 4.37 | 3 |
| 332 | 810785 | AA481757 | 0.25 | 1.22 | 4.79 | 3.85 | 4.29 | 6.23 | 3 |
| 333 | 306146 | N90523 | 1.02 | 4.83 | 4.75 | 5.62 | 3.94 | 4.69 | 3 |
| 334 | 283751 | N50745 | 1.20 | 5.71 | 4.75 | 7.29 | 3.35 | 3.60 | 3 |
| 335 | 1467026 | AA883127 | 0.09 | 0.45 | 4.71 | 4.52 | 4.33 | 5.29 | 3 |
| 336 | 1056200 | AA621019 | 0.84 | 3.94 | 4.71 | 3.82 | 7.24 | 3.08 | 3 |
| 337 | 781341 | AA448396 | 6.94 | 32.63 | 4.70 | 6.91 | 4.15 | 3.05 | 3 |
| 338 | 292068 | N73301 | 0.78 | 3.65 | 4.70 | 5.91 | 4.89 | 3.31 | 3 |
| 339 | 249949 | H97000 | 0.04 | 0.19 | 4.69 | 3.56 | 3.95 | 6.55 | 3 |
| 340 | 66406 | T66936 | 0.74 | 3.48 | 4.67 | 5.15 | 3.86 | 5.01 | 3 |
| 341 | 376764 | AA046321 | 0.36 | 1.69 | 4.65 | 5.09 | 5.23 | 3.64 | 3 |
| 342 | 52543 | H23482 | 0.73 | 3.36 | 4.63 | 3.32 | 7.07 | 3.51 | 3 |
| 343 | 461775 | AA682405 | 0.09 | 0.43 | 4.63 | 4.03 | 4.94 | 4.93 | 3 |
| 344 | 544664 | AA074666 | 4.89 | 22.62 | 4.63 | 3.06 | 3.22 | 7.60 | 3 |
| 345 | 595109 | AA173926 | 2.40 | 11.07 | 4.60 | 3.28 | 5.08 | 5.45 | 3 |
| 346 | 292933 | N69491 | 0.67 | 3.08 | 4.60 | 4.85 | 3.14 | 5.80 | 3 |
| 347 | 287749 | N62245 | 1.77 | 8.14 | 4.60 | 4.35 | 3.56 | 5.89 | 3 |
| 348 | 461235 | AA699762 | 0.22 | 0.99 | 4.58 | 6.38 | 4.05 | 3.31 | 3 |
| 349 | 295551 | N74956 | 15.03 | 68.72 | 4.57 | 3.02 | 5.75 | 4.95 | 3 |
| 350 | 186918 | H43317 | 0.01 | 0.04 | 4.56 | 5.14 | 4.81 | 3.75 | 3 |
| 351 | 782800 | AA448184 | 5.53 | 25.23 | 4.56 | 4.05 | 3.27 | 6.37 | 3 |
| 352 | 296155 | W00895 | 1.25 | 5.67 | 4.55 | 4.56 | 3.51 | 5.59 | 3 |
| 353 | 282720 | N50079 | 3.70 | 16.81 | 4.54 | 3.79 | 4.39 | 5.44 | 3 |
| 354 | 588840 | AA157787 | 3.00 | 13.59 | 4.53 | 3.16 | 5.08 | 5.34 | 3 |
| 355 | 509479 | AA056395 | 4.33 | 19.58 | 4.52 | 3.27 | 6.13 | 4.18 | 3 |
| 356 | 413071 | AA707794 | 0.13 | 0.60 | 4.52 | 3.14 | 3.41 | 7.01 | 3 |
| 357 | 39798 | R53294 | 0.85 | 3.85 | 4.51 | 5.43 | 3.36 | 4.75 | 3 |
| 358 | 970591 | AA683085 | 29.84 | 133.80 | 4.48 | 3.35 | 5.80 | 4.30 | 3 |
| 359 | 814765 | AA454947 | 2.35 | 10.51 | 4.48 | 5.20 | 5.13 | 3.12 | 3 |
| 360 | 490548 | AA126799 | 1.22 | 5.43 | 4.46 | 5.70 | 4.50 | 3.19 | 3 |
| 361 | 795371 | AA453273 | 2.35 | 10.45 | 4.45 | 4.16 | 5.42 | 3.76 | 3 |
| 362 | 358457 | W96114 | 7.33 | 32.61 | 4.45 | 3.43 | 5.36 | 4.54 | 3 |
| 363 | 356732 | W84486 | 0.67 | 2.96 | 4.43 | 4.85 | 4.44 | 4.00 | 3 |
| 364 | 1627623 | AI014704 | 0.49 | 2.19 | 4.43 | 5.61 | 3.81 | 3.87 | 3 |
| 365 | 341942 | W60015 | 2.28 | 10.04 | 4.40 | 3.32 | 4.59 | 5.27 | 3 |
| 366 | 842906 | AA486430 | 1.04 | 4.57 | 4.39 | 3.03 | 3.33 | 6.82 | 3 |
| 367 | 1475463 | AA857804 | 1.80 | 7.88 | 4.39 | 5.77 | 3.14 | 4.26 | 3 |
| 368 | 359135 | AA010128 | 0.47 | 2.06 | 4.38 | 3.23 | 3.76 | 6.16 | 3 |
| 369 | 198104 | R94633 | 0.21 | 0.93 | 4.38 | 4.72 | 3.33 | 5.10 | 3 |
| 370 | 249562 | H84926 | 0.04 | 0.18 | 4.37 | 3.06 | 5.05 | 4.99 | 3 |
| 371 | 308466 | N95495 | 2.29 | 9.99 | 4.36 | 5.17 | 4.31 | 3.59 | 3 |
| 372 | 324313 | AA284108 | 0.02 | 0.09 | 4.34 | 3.11 | 6.72 | 3.19 | 3 |
| 373 | 280342 | N49261 | 4.13 | 17.91 | 4.33 | 3.32 | 4.21 | 5.47 | 3 |
| 374 | 40881 | R56055 | 0.15 | 0.64 | 4.32 | 4.06 | 4.72 | 4.18 | 3 |
| 375 | 250519 | H89698 | 1.26 | 5.41 | 4.31 | 4.10 | 3.51 | 5.33 | 3 |
| 376 | 418150 | W90154 | 0.62 | 2.65 | 4.31 | 3.30 | 6.39 | 3.24 | 3 |
| 377 | 258761 | N30185 | 1.26 | 5.44 | 4.30 | 4.24 | 3.24 | 5.43 | 3 |
| 378 | 25274 | R12804 | 0.39 | 1.66 | 4.29 | 3.40 | 5.79 | 3.69 | 3 |
| 379 | 684835 | AA251826 | 0.63 | 2.72 | 4.29 | 3.87 | 4.70 | 4.31 | 3 |
| 380 | 417855 | W88792 | 1.93 | 8.22 | 4.27 | 3.03 | 4.54 | 5.24 | 3 |
| 381 | 1556056 | AA975388 | 0.19 | 0.82 | 4.25 | 3.90 | 3.93 | 4.93 | 3 |
| 382 | 502767 | AA126259 | 0.42 | 1.80 | 4.25 | 3.90 | 3.05 | 5.79 | 3 |
| 383 | 453599 | AA679569 | 1.66 | 7.01 | 4.23 | 6.00 | 3.07 | 3.61 | 3 |
| 384 | 42793 | R59722 | 0.30 | 1.26 | 4.22 | 3.06 | 5.07 | 4.55 | 3 |
| 385 | 183771 | H44448 | 2.36 | 9.98 | 4.22 | 3.65 | 5.59 | 3.43 | 3 |
| 386 | 365157 | AA024637 | 2.33 | 9.83 | 4.22 | 3.26 | 5.64 | 3.75 | 3 |
| 387 | 324255 | W47254 | 1.68 | 7.07 | 4.21 | 3.45 | 4.51 | 4.67 | 3 |
| 388 | 809421 | AA459909 | 3.57 | 15.01 | 4.20 | 3.52 | 5.97 | 3.12 | 3 |
| 389 | 301867 | N92478 | 0.36 | 1.52 | 4.20 | 3.30 | 4.63 | 4.67 | 3 |
| 390 | 825089 | AA489247 | 0.11 | 0.45 | 4.19 | 3.43 | 4.75 | 4.40 | 3 |
| 391 | 365641 | AA025937 | 1.29 | 5.38 | 4.19 | 3.00 | 3.59 | 5.97 | 3 |
| 392 | 76196 | T59668 | 2.26 | 9.46 | 4.18 | 3.30 | 5.98 | 3.26 | 3 |
| 393 | 788566 | AA452966 | 1.36 | 5.68 | 4.18 | 3.56 | 3.76 | 5.22 | 3 |
| 394 | 755301 | AA496360 | 0.05 | 0.22 | 4.16 | 3.57 | 3.72 | 5.18 | 3 |
| 395 | 1387760 | AA838691 | 1.66 | 6.85 | 4.14 | 4.66 | 4.62 | 3.14 | 3 |
| 396 | 510170 | AA053129 | 6.42 | 26.51 | 4.13 | 3.38 | 4.00 | 5.01 | 3 |
| 397 | 127925 | R08935 | 0.28 | 1.14 | 4.13 | 3.18 | 4.98 | 4.22 | 3 |
| 398 | 845380 | AA644099 | 1.43 | 5.90 | 4.12 | 4.14 | 4.91 | 3.31 | 3 |
| 399 | 305538 | N89861 | 5.14 | 21.16 | 4.11 | 4.96 | 4.11 | 3.26 | 3 |
| 400 | 548693 | AA125825 | 0.49 | 2.02 | 4.11 | 4.24 | 3.39 | 4.71 | 3 |
| 401 | 125183 | R05693 | 7.53 | 30.82 | 4.09 | 3.66 | 3.99 | 4.63 | 3 |
| 402 | 289945 | N64617 | 0.64 | 2.60 | 4.08 | 3.25 | 3.58 | 5.40 | 3 |
| 403 | 431803 | AA678021 | 17.87 | 72.78 | 4.07 | 3.72 | 5.27 | 3.23 | 3 |
| 404 | 824739 | AA489000 | 1.22 | 4.98 | 4.07 | 3.33 | 4.59 | 4.30 | 3 |
| 405 | 136180 | R33303 | 2.09 | 8.49 | 4.07 | 3.09 | 4.10 | 5.02 | 3 |
| 406 | 51469 | H23985 | 2.11 | 8.55 | 4.05 | 3.08 | 3.13 | 5.93 | 3 |
| 407 | 461592 | AA705142 | 2.49 | 10.05 | 4.04 | 5.90 | 3.14 | 3.07 | 3 |
| 408 | 290378 | N64508 | 0.80 | 3.22 | 4.03 | 3.72 | 3.29 | 5.07 | 3 |
| 409 | 795830 | AA460571 | 0.08 | 0.34 | 4.00 | 3.83 | 4.74 | 3.44 | 3 |
| 410 | 416390 | W86860 | 2.29 | 9.11 | 3.99 | 3.72 | 3.60 | 4.63 | 3 |
| 411 | 150135 | H01926 | 4.15 | 16.49 | 3.97 | 3.31 | 4.75 | 3.86 | 3 |
| 412 | 47681 | H11792 | 8.31 | 32.97 | 3.97 | 3.15 | 3.42 | 5.33 | 3 |
| 413 | 271006 | N29901 | 3.83 | 15.20 | 3.96 | 3.30 | 5.01 | 3.59 | 3 |
| 414 | 625764 | AA188366 | 0.91 | 3.60 | 3.96 | 4.14 | 4.14 | 3.60 | 3 |
| 415 | 1654974 | A1023724 | 2.04 | 8.02 | 3.94 | 3.65 | 3.46 | 4.71 | 3 |
| 416 | 194305 | H50656 | 0.29 | 1.14 | 3.93 | 4.53 | 3.70 | 3.55 | 3 |
| 417 | 32870 | R43205 | 0.34 | 1.32 | 3.93 | 3.19 | 4.45 | 4.15 | 3 |
| 418 | 786083 | AA448676 | 2.27 | 8.89 | 3.93 | 4.14 | 4.07 | 3.57 | 3 |
| 419 | 613126 | AA211448 | 0.43 | 1.69 | 3.92 | 4.47 | 3.83 | 3.47 | 3 |
| 420 | 293576 | N69283 | 4.31 | 16.90 | 3.92 | 3.28 | 3.75 | 4.72 | 3 |
| 421 | 115230 | T86527 | 0.02 | 0.09 | 3.86 | 4.33 | 3.12 | 4.12 | 3 |
| 422 | 754355 | AA436152 | 3.05 | 11.74 | 3.85 | 5.38 | 3.09 | 3.09 | 3 |
| 423 | 77244 | T50139 | 2.66 | 10.26 | 3.85 | 4.86 | 3.02 | 3.67 | 3 |
| 424 | 813675 | AA453832 | 2.90 | 11.13 | 3.84 | 4.81 | 3.36 | 3.36 | 3 |
| 425 | 295710 | N72697 | 1.17 | 4.47 | 3.82 | 4.55 | 3.33 | 3.58 | 3 |
| 426 | 884690 | AA630016 | 0.01 | 0.04 | 3.74 | 3.23 | 4.39 | 3.59 | 3 |
| 427 | 377884 | AA777092 | 0.25 | 0.95 | 3.74 | 3.07 | 3.48 | 4.65 | 3 |
| 428 | 453642 | AA775888 | 0.09 | 0.34 | 3.68 | 3.83 | 4.03 | 3.19 | 3 |
| 429 | 1586340 | AA974348 | 2.85 | 10.51 | 3.68 | 4.08 | 3.46 | 3.51 | 3 |
| 430 | 757404 | AA426341 | 5.69 | 20.94 | 3.68 | 3.33 | 3.85 | 3.86 | 3 |
| 431 | 745286 | AA625558 | 0.16 | 0.59 | 3.66 | 3.24 | 3.30 | 4.45 | 3 |
| 432 | 773599 | AA429422 | 2.41 | 8.80 | 3.66 | 3.28 | 4.40 | 3.28 | 3 |
| 433 | 487723 | AA043552 | 1.57 | 5.73 | 3.66 | 3.48 | 4.23 | 3.25 | 3 |
| 434 | 288658 | N79353 | 0.26 | 0.95 | 3.65 | 4.71 | 3.21 | 3.02 | 3 |
| 435 | 279920 | N57526 | 0.69 | 2.51 | 3.64 | 4.05 | 3.12 | 3.76 | 3 |
| 436 | 813648 | AA453679 | 3.91 | 14.21 | 3.64 | 3.48 | 4.25 | 3.19 | 3 |
| 437 | 136744 | R35230 | 2.00 | 7.22 | 3.62 | 3.22 | 3.26 | 4.37 | 3 |
| 438 | 487425 | AA046523 | 2.77 | 10.00 | 3.61 | 3.70 | 3.44 | 3.70 | 3 |
| 439 | 589479 | AA157129 | 4.22 | 15.14 | 3.58 | 3.96 | 3.72 | 3.07 | 3 |
| 440 | 256260 | H94617 | 1.40 | 4.95 | 3.54 | 3.05 | 3.96 | 3.62 | 3 |
| 441 | 627273 | AA191461 | 1.01 | 3.54 | 3.52 | 3.59 | 3.73 | 3.24 | 3 |
| 442 | 1604596 | AA996230 | 0.80 | 2.80 | 3.49 | 3.29 | 3.84 | 3.32 | 3 |
| 443 | 843224 | AA488445 | 5.65 | 19.64 | 3.47 | 3.47 | 3.36 | 3.59 | 3 |
| 444 | 781339 | AA448394 | 0.19 | 0.64 | 3.42 | 3.04 | 4.21 | 3.02 | 3 |
| 445 | 275653 | R93309 | 0.72 | 2.45 | 3.41 | 3.42 | 3.22 | 3.60 | 3 |
| 446 | 840384 | AA485773 | 5.81 | 19.81 | 3.41 | 3.01 | 3.43 | 3.80 | 3 |
| 447 | 412949 | AA707840 | 1.07 | 3.62 | 3.39 | 3.46 | 3.12 | 3.59 | 3 |
| 448 | 731044 | AA421273 | 1.36 | 4.57 | 3.37 | 3.82 | 3.16 | 3.13 | 3 |
| 449 | 501731 | AA127861 | 2.15 | 6.95 | 3.23 | 3.08 | 3.55 | 3.05 | 3 |
| 450 | 1640931 | AI024401 | 0.21 | 0.66 | 3.13 | 3.14 | 3.07 | 3.17 | 3 |
| 451 | 299559 | N74995 | 0.01 | 9.92 | 991.80 | 1.38 | 2966.06 | 7.96 | 2 |
| 452 | 365551 | AA009615 | 0.01 | 8.39 | 838.64 | 1.96 | 2506.90 | 7.05 | 2 |
| 453 | 42627 | R60995 | 0.01 | 9.14 | 660.46 | 2.32 | 61.14 | 1917.92 | 2 |
| 454 | 277327 | N57483 | 0.01 | 9.00 | 613.07 | 1.58 | 1831.96 | 5.67 | 2 |
| 455 | 773640 | AA433885 | 0.01 | 5.08 | 508.28 | 2.41 | 1516.35 | 6.10 | 2 |
| 456 | 121863 | 797352 | 0.04 | 18.56 | 445.85 | 3.24 | 1331.89 | 2.42 | 2 |
| 457 | 764291 | AA479498 | 0.01 | 4.34 | 433.98 | 2.48 | 1294.73 | 4.72 | 2 |
| 458 | 290213 | N64379 | 0.01 | 3.94 | 393.88 | 0.73 | 1171.33 | 9.57 | 2 |
| 459 | 781157 | AA429904 | 0.01 | 2.83 | 283.26 | 1.00 | 841.07 | 7.73 | 2 |
| 460 | 781036 | AA446446 | 0.01 | 1.70 | 228.86 | 10.58 | 674.66 | 1.35 | 2 |
| 461 | 784216 | AA446866 | 0.01 | 1.58 | 157.55 | 1.61 | 467.71 | 3.31 | 2 |
| 462 | 1377071 | AA812676 | 0.01 | 1.54 | 154.10 | 3.95 | 457.34 | 1.00 | 2 |
| 463 | 609935 | AA169154 | 0.02 | 2.74 | 114.55 | 1.41 | 338.20 | 4.03 | 2 |
| 464 | 203878 | H56453 | 0.02 | 1.93 | 95.78 | 0.80 | 140.40 | 146.14 | 2 |
| 465 | 815794 | AA485214 | 0.08 | 6.83 | 85.65 | 0.89 | 150.46 | 105.59 | 2 |
| 466 | 347182 | W80611 | 0.01 | 0.82 | 81.58 | 1.42 | 229.57 | 13.76 | 2 |
| 467 | 611373 | AA176833 | 0.01 | 0.71 | 70.86 | 2.42 | 205.80 | 4.35 | 2 |
| 468 | 746152 | AA419486 | 0.08 | 5.16 | 65.49 | 0.87 | 95.72 | 99.88 | 2 |
| 469 | 395708 | AA757754 | 0.01 | 0.83 | 60.41 | 6.88 | 173.62 | 0.72 | 2 |
| 470 | 684879 | AA251784 | 0.05 | 2.87 | 58.88 | 1.03 | 51.92 | 123.70 | 2 |
| 471 | 344589 | W73144 | 0.22 | 12.41 | 56.02 | 78.84 | 88.50 | 0.72 | 2 |
| 472 | 582811 | AA086475 | 0.01 | 0.28 | 53.90 | 5.36 | 154.39 | 1.93 | 2 |
| 473 | 30207 | R40231 | 0.04 | 2.21 | 52.11 | 0.54 | 150.40 | 5.39 | 2 |
| 474 | 1358266 | AA873635 | 0.08 | 3.95 | 51.39 | 1.05 | 149.73 | 3.40 | 2 |
| 475 | 757435 | AA437224 | 1.20 | 60.94 | 50.82 | 144.61 | 1.37 | 6.47 | 2 |
| 476 | 757143 | AA443936 | 0.19 | 9.88 | 50.76 | 4.68 | 145.07 | 2.54 | 2 |
| 477 | 33827 | R44741 | 0.02 | 1.10 | 50.72 | 0.63 | 139.36 | 12.17 | 2 |
| 478 | 825740 | AA504844 | 0.26 | 13.02 | 49.67 | 0.91 | 56.58 | 91.51 | 2 |
| 479 | 813748 | AA453802 | 0.01 | 0.49 | 49.10 | 3.38 | 142.92 | 1.00 | 2 |
| 480 | 1505360 | AA905896 | 0.26 | 12.64 | 47.88 | 137.81 | 2.72 | 3.10 | 2 |
| 481 | 785849 | AA449120 | 0.01 | 0.60 | 45.99 | 2.24 | 128.30 | 7.45 | 2 |
| 482 | 43101 | R59936 | 0.01 | 0.46 | 45.56 | 1.92 | 130.70 | 4.06 | 2 |
| 483 | 714498 | AA292429 | 0.02 | 0.87 | 44.45 | 4.84 | 125.85 | 2.84 | 2 |
| 484 | 811121 | AA485688 | 0.01 | 0.44 | 44.03 | 3.52 | 127.57 | 1.00 | 2 |
| 485 | 743362 | AA400385 | 0.04 | 1.89 | 43.31 | 2.77 | 123.35 | 3.82 | 2 |
| 486 | 1435339 | AA857748 | 0.01 | 0.38 | 37.79 | 5.72 | 105.49 | 2.18 | 2 |
| 487 | 28422 | R14230 | 0.46 | 17.33 | 37.42 | 0.83 | 107.04 | 4.40 | 2 |
| 488 | 530608 | AA071089 | 0.01 | 0.38 | 36.23 | 3.89 | 103.70 | 1.11 | 2 |
| 489 | 774446 | AA446120 | 0.05 | 1.69 | 36.22 | 1.20 | 103.43 | 4.04 | 2 |
| 490 | 428828 | AA005254 | 0.01 | 0.35 | 35.29 | 1.77 | 100.95 | 3.14 | 2 |
| 491 | 196544 | R91566 | 0.25 | 8.56 | 34.73 | 0.93 | 52.03 | 51.22 | 2 |
| 492 | 592928 | AA159179 | 0.18 | 6.00 | 33.64 | 1.12 | 96.63 | 3.18 | 2 |
| 493 | 773083 | AA425307 | 2.25 | 73.55 | 32.64 | 1.02 | 93.52 | 3.37 | 2 |
| 494 | 37823 | R59473 | 0.03 | 1.12 | 32.46 | 1.79 | 89.38 | 6.23 | 2 |
| 495 | 49858 | H29290 | 0.08 | 2.62 | 31.22 | 87.92 | 0.12 | 5.61 | 2 |
| 496 | 743189 | AA401434 | 0.02 | 0.70 | 30.58 | 2.89 | 81.20 | 7.60 | 2 |
| 497 | 815861 | AA485052 | 0.26 | 7.89 | 30.35 | 0.77 | 34.54 | 55.73 | 2 |
| 498 | 489656 | AA099369 | 0.01 | 0.30 | 30.08 | 1.00 | 7.97 | 81.28 | 2 |
| 499 | 38347 | R49439 | 0.21 | 5.88 | 27.65 | 1.51 | 76.38 | 5.06 | 2 |
| 500 | 167205 | R90934 | 0.58 | 16.16 | 27.63 | 72.37 | 9.84 | 0.70 | 2 |
| 501 | 53039 | R15740 | 0.01 | 0.27 | 27.34 | 0.74 | 70.25 | 11.03 | 2 |
| 502 | 796549 | AA460274 | 0.01 | 0.27 | 27.13 | 1.21 | 69.04 | 11.15 | 2 |
| 503 | 771294 | AA443624 | 0.01 | 0.20 | 27.04 | 8.33 | 1.36 | 71.43 | 2 |
| 504 | 194318 | H50655 | 0.53 | 14.38 | 26.99 | 0.69 | 27.83 | 52.45 | 2 |
| 505 | 769000 | AA425158 | 0.01 | 0.26 | 28.26 | 5.77 | 72.00 | 1.00 | 2 |
| 506 | 280699 | N47445 | 0.55 | 14.15 | 25.90 | 2.28 | 11.75 | 63.66 | 2 |
| 507 | 283379 | N52767 | 0.02 | 0.39 | 25.65 | 0.59 | 66.04 | 10.31 | 2 |
| 508 | 27605 | R40018 | 0.07 | 1.90 | 25.57 | 2.05 | 68.60 | 6.05 | 2 |
| 509 | 530958 | AA070437 | 0.01 | 0.25 | 25.21 | 2.29 | 67.88 | 5.46 | 2 |
| 510 | 436121 | AA701996 | 0.02 | 0.60 | 25.17 | 3.88 | 71.30 | 0.34 | 2 |
| 511 | 53315 | R15813 | 0.01 | 0.25 | 25.10 | 2.87 | 64.40 | 8.05 | 2 |
| 512 | 357884 | W94486 | 0.01 | 0.25 | 24.95 | 2.32 | 41.17 | 31.35 | 2 |
| 513 | 172517 | H19826 | 1.00 | 24.91 | 24.86 | 2.27 | 26.82 | 45.50 | 2 |
| 514 | 251806 | H96647 | 0.01 | 0.24 | 24.50 | 1.76 | 60.48 | 11.25 | 2 |
| 515 | 80643 | T57803 | 1.09 | 26.54 | 24.25 | 0.44 | 9.54 | 62.76 | 2 |
| 516 | 824025 | AA490945 | 0.31 | 7.41 | 24.24 | 0.95 | 28.65 | 43.13 | 2 |
| 517 | 825606 | AA504625 | 0.15 | 3.51 | 23.68 | 0.87 | 21.91 | 48.25 | 2 |
| 518 | 839882 | AA490044 | 0.28 | 6.50 | 23.31 | 0.87 | 64.54 | 4.51 | 2 |
| 519 | 81050 | T70198 | 0.01 | 0.23 | 23.00 | 1.00 | 42.67 | 25.34 | 2 |
| 520 | 220655 | H88143 | 0.01 | 0.30 | 22.12 | 3.92 | 61.72 | 0.73 | 2 |
| 521 | 591055 | AA161097 | 0.02 | 0.34 | 21.79 | 2.34 | 9.53 | 53.51 | 2 |
| 522 | 824270 | AA491261 | 0.17 | 3.65 | 21.71 | 0.88 | 10.05 | 54.20 | 2 |
| 523 | 46843 | H10072 | 0.01 | 0.14 | 21.70 | 39.83 | 1.50 | 23.77 | 2 |
| 524 | 358316 | W95757 | 0.02 | 0.51 | 21.13 | 0.72 | 58.79 | 3.87 | 2 |
| 525 | 267085 | N24848 | 0.05 | 0.95 | 20.75 | 0.93 | 57.89 | 3.43 | 2 |
| 526 | 686552 | AA255954 | 0.30 | 6.19 | 20.39 | 0.93 | 42.76 | 17.48 | 2 |
| 527 | 32962 | R43910 | 0.05 | 1.02 | 20.24 | 56.76 | 0.20 | 3.78 | 2 |
| 528 | 199337 | R95684 | 0.20 | 3.94 | 20.04 | 1.29 | 30.54 | 28.27 | 2 |
| 529 | 795831 | AA461508 | 0.01 | 0.20 | 19.97 | 1.00 | 38.72 | 20.17 | 2 |
| 530 | 826256 | AA520979 | 0.17 | 3.26 | 19.61 | 1.10 | 7.58 | 50.17 | 2 |
| 531 | 197265 | R86970 | 0.01 | 0.20 | 19.50 | 2.79 | 10.31 | 45.41 | 2 |
| 532 | 162310 | H28091 | 0.17 | 3.30 | 19.47 | 2.35 | 30.81 | 25.26 | 2 |
| 533 | 759163 | AA496022 | 0.01 | 0.19 | 19.37 | 7.38 | 1.00 | 49.74 | 2 |
| 534 | 809583 | AA456628 | 0.01 | 0.19 | 19.26 | 9.69 | 1.93 | 46.16 | 2 |
| 535 | 80338 | T65736 | 1.11 | 21.01 | 18.93 | 41.41 | 14.31 | 1.08 | 2 |
| 536 | 1049284 | AA620747 | 0.04 | 0.74 | 16.21 | 2.02 | 47.06 | 5.54 | 2 |
| 537 | 839048 | AA487505 | 0.19 | 3.45 | 17.78 | 1.21 | 45.91 | 6.22 | 2 |
| 538 | 42907 | R60020 | 0.03 | 0.54 | 17.62 | 1.87 | 45.44 | 5.54 | 2 |
| 539 | 361899 | AA001376 | 0.01 | 0.17 | 17.44 | 1.97 | 34.15 | 16.22 | 2 |
| 540 | 823727 | AA489662 | 0.03 | 0.44 | 17.25 | 2.37 | 40.61 | 8.77 | 2 |
| 541 | 1475476 | AA857809 | 0.51 | 8.76 | 17.22 | 46.76 | 3.64 | 1.27 | 2 |
| 542 | 1410444 | AA857163 | 0.06 | 1.06 | 17.19 | 1.87 | 37.48 | 12.21 | 2 |
| 543 | 756629 | AA481481 | 0.01 | 0.11 | 16.78 | 14.65 | 1.51 | 34.18 | 2 |
| 544 | 266366 | N26559 | 0.02 | 0.29 | 16.62 | 2.86 | 39.78 | 7.22 | 2 |
| 545 | 283070 | N51297 | 0.07 | 1.14 | 16.47 | 2.84 | 40.90 | 5.65 | 2 |
| 546 | 825726 | AA504838 | 0.19 | 2.94 | 15.84 | 2.63 | 16.52 | 28.36 | 2 |
| 547 | 746232 | AA417713 | 0.38 | 5.98 | 15.64 | 0.69 | 21.09 | 25.15 | 2 |
| 548 | 647679 | AA206311 | 0.17 | 2.60 | 15.42 | 1.59 | 41.42 | 3.27 | 2 |
| 549 | 291947 | N73083 | 0.06 | 0.95 | 15.23 | 3.92 | 41.20 | 0.57 | 2 |
| 550 | 811766 | AA463445 | 0.26 | 3.91 | 15.03 | 1.53 | 28.43 | 15.13 | 2 |
| 551 | 51599 | H18932 | 0.26 | 3.94 | 14.95 | 0.42 | 26.10 | 18.34 | 2 |
| 552 | 37205 | R49592 | 0.09 | 1.38 | 14.69 | 2.32 | 38.63 | 3.12 | 2 |
| 553 | 364865 | AA035745 | 0.04 | 0.54 | 14.60 | 36.64 | 0.27 | 6.87 | 2 |
| 554 | 1455603 | AA863125 | 0.38 | 5.51 | 14.53 | 3.43 | 38.09 | 2.07 | 2 |
| 555 | 770860 | AA434388 | 0.01 | 0.14 | 14.40 | 1.00 | 18.93 | 23.25 | 2 |
| 556 | 795347 | AA453260 | 0.03 | 0.42 | 14.24 | 3.59 | 36.42 | 2.72 | 2 |
| 557 | 30082 | R40105 | 0.17 | 2.42 | 14.20 | 1.58 | 37.99 | 3.04 | 2 |
| 558 | 430237 | AA010224 | 0.05 | 0.77 | 14.13 | 1.89 | 35.90 | 4.61 | 2 |
| 559 | 284001 | N53380 | 0.88 | 12.46 | 14.13 | 36.41 | 2.96 | 3.01 | 2 |
| 560 | 436402 | AA699601 | 0.16 | 2.19 | 14.09 | 0.88 | 36.28 | 5.10 | 2 |
| 561 | 418004 | W90705 | 0.30 | 4.17 | 13.89 | 2.99 | 11.04 | 27.66 | 2 |
| 562 | 26806 | R37738 | 0.10 | 1.31 | 13.68 | 2.42 | 26.44 | 12.18 | 2 |
| 563 | 771004 | AA427719 | 0.29 | 3.95 | 13.50 | 0.64 | 21.58 | 18.27 | 2 |
| 564 | 49499 | H15549 | 0.63 | 8.41 | 13.36 | 2.08 | 8.82 | 29.19 | 2 |
| 565 | 284288 | N52193 | 0.01 | 0.08 | 13.00 | 10.97 | 1.54 | 26.50 | 2 |
| 566 | 364844 | AA035730 | 0.03 | 0.36 | 12.97 | 30.80 | 0.76 | 7.37 | 2 |
| 567 | 361122 | AA017379 | 0.01 | 0.13 | 12.88 | 0.65 | 12.28 | 25.70 | 2 |
| 568 | 1526789 | AA911236 | 0.65 | 8.32 | 12.81 | 1.86 | 8.17 | 28.39 | 2 |
| 569 | 33066 | R43915 | 0.06 | 0.71 | 12.79 | 7.14 | 29.01 | 2.23 | 2 |
| 570 | 530954 | AA070435 | 0.16 | 1.99 | 12.64 | 1.33 | 31.49 | 5.10 | 2 |
| 571 | 47204 | H11051 | 0.03 | 0.33 | 12.63 | 2.47 | 31.19 | 4.25 | 2 |
| 572 | 795446 | AA453616 | 0.01 | 0.13 | 12.56 | 22.57 | 14.55 | 0.58 | 2 |
| 573 | 757428 | AA437212 | 0.40 | 4.99 | 12.41 | 1.18 | 29.00 | 7.04 | 2 |
| 574 | 1049185 | AA620697 | 0.76 | 9.44 | 12.39 | 10.40 | 0.39 | 26.39 | 2 |
| 575 | 364352 | AA022496 | 0.01 | 0.12 | 12.38 | 1.96 | 14.19 | 20.98 | 2 |
| 576 | 47264 | H10713 | 0.30 | 3.72 | 12.36 | 2.11 | 23.87 | 11.09 | 2 |
| 577 | 71671 | T57927 | 0.01 | 0.12 | 12.24 | 6.29 | 29.43 | 1.00 | 2 |
| 578 | 812977 | AA464617 | 1.53 | 18.67 | 12.17 | 29.85 | 2.25 | 4.42 | 2 |
| 579 | 195845 | R92201 | 0.14 | 1.69 | 12.02 | 1.00 | 14.95 | 20.10 | 2 |
| 580 | 728797 | AA398321 | 0.04 | 0.52 | 11.95 | 2.42 | 30.06 | 3.37 | 2 |
| 581 | 739450 | AA477227 | 1.99 | 23.53 | 11.82 | 2.08 | 17.13 | 16.24 | 2 |
| 582 | 324494 | W51795 | 0.01 | 0.12 | 11.78 | 2.89 | 4.46 | 28.00 | 2 |
| 583 | 796367 | AA456143 | 0.01 | 0.12 | 11.74 | 0.81 | 30.95 | 3.44 | 2 |
| 584 | 591157 | AA161188 | 0.02 | 0.18 | 11.71 | 0.58 | 20.54 | 14.01 | 2 |
| 585 | 345262 | W72881 | 0.01 | 0.12 | 11.47 | 4.71 | 0.92 | 28.79 | 2 |
| 586 | 50982 | H18015 | 0.06 | 0.70 | 11.45 | 1.92 | 28.84 | 3.60 | 2 |
| 587 | 753984 | AA478952 | 0.66 | 7.46 | 11.23 | 2.83 | 27.04 | 3.82 | 2 |
| 588 | 1031561 | AA609300 | 0.22 | 2.42 | 11.22 | 1.36 | 28.86 | 3.43 | 2 |
| 589 | 592728 | AA160670 | 0.03 | 0.34 | 11.17 | 0.33 | 16.85 | 16.34 | 2 |
| 590 | 770854 | AA427737 | 0.09 | 0.95 | 11.06 | 2.56 | 22.78 | 7.83 | 2 |
| 591 | 795526 | AA454222 | 0.12 | 1.32 | 10.94 | 1.10 | 4.45 | 27.27 | 2 |
| 592 | 271483 | N31808 | 0.01 | 0.11 | 10.91 | 3.77 | 1.00 | 27.95 | 2 |
| 593 | 193333 | H48070 | 0.36 | 3.90 | 10.90 | 0.92 | 17.96 | 13.80 | 2 |
| 594 | 813738 | AA453787 | 0.03 | 0.36 | 10.89 | 1.90 | 26.77 | 4.00 | 2 |
| 595 | 41869 | R66438 | 0.10 | 1.12 | 10.87 | 2.21 | 25.57 | 4.84 | 2 |
| 596 | 753428 | AA410434 | 0.57 | 6.12 | 10.84 | 0.51 | 15.37 | 16.62 | 2 |
| 597 | 298417 | N74131 | 0.03 | 0.31 | 10.83 | 2.09 | 20.21 | 10.20 | 2 |
| 598 | 287190 | N66900 | 0.29 | 3.13 | 10.82 | 1.79 | 27.39 | 3.26 | 2 |
| 599 | 257955 | N30751 | 0.86 | 9.32 | 10.79 | 2.05 | 10.22 | 20.10 | 2 |
| 600 | 433155 | AA680136 | 0.01 | 0.11 | 10.76 | 22.91 | 1.00 | 8.36 | 2 |
| 601 | 26164 | R39765 | 0.75 | 7.96 | 10.65 | 2.40 | 25.25 | 4.31 | 2 |
| 602 | 824802 | AA489073 | 0.77 | 8.09 | 10.50 | 0.74 | 8.99 | 21.77 | 2 |
| 603 | 797055 | AA463248 | 0.72 | 7.51 | 10.47 | 1.14 | 25.88 | 4.40 | 2 |
| 604 | 448038 | AA702803 | 1.83 | 18.95 | 10.38 | 25.10 | 1.37 | 4.66 | 2 |
| 605 | 731095 | AA421171 | 1.22 | 12.67 | 10.36 | 4.91 | 23.39 | 2.78 | 2 |
| 606 | 307138 | N93721 | 0.04 | 0.44 | 10.36 | 2.67 | 21.63 | 6.78 | 2 |
| 607 | 768043 | AA418852 | 0.02 | 0.21 | 10.35 | 2.40 | 24.49 | 4.17 | 2 |
| 608 | 712401 | AA281784 | 0.08 | 0.87 | 10.32 | 0.88 | 14.48 | 15.60 | 2 |
| 609 | 489509 | AA099148 | 0.45 | 4.59 | 10.32 | 2.87 | 24.58 | 3.50 | 2 |
| 610 | 203184 | H54451 | 0.41 | 4.21 | 10.31 | 0.86 | 6.31 | 23.76 | 2 |
| 611 | 277621 | N49389 | 0.54 | 5.55 | 10.21 | 0.77 | 14.23 | 15.64 | 2 |
| 612 | 729953 | AA412049 | 0.93 | 9.47 | 10.19 | 22.87 | 7.21 | 0.48 | 2 |
| 613 | 590640 | AA158035 | 0.01 | 0.12 | 10.11 | 11.51 | 17.15 | 1.65 | 2 |
| 614 | 868838 | AA775223 | 0.81 | 8.21 | 10.10 | 3.45 | 25.02 | 1.83 | 2 |
| 615 | 197651 | R94504 | 0.25 | 2.53 | 10.07 | 0.93 | 11.85 | 17.42 | 2 |
| 616 | 740457 | AA478036 | 1.60 | 16.05 | 10.05 | 2.67 | 6.06 | 21.42 | 2 |
| 617 | 745248 | AA626167 | 0.24 | 2.42 | 9.97 | 8.61 | 20.18 | 1.12 | 2 |
| 618 | 855391 | AA664007 | 0.23 | 2.29 | 9.95 | 1.40 | 12.15 | 16.30 | 2 |
| 619 | 781110 | AA430042 | 0.01 | 0.10 | 9.90 | 4.50 | 24.20 | 1.00 | 2 |
| 620 | 725877 | AA292226 | 0.50 | 4.94 | 9.89 | 0.67 | 13.69 | 15.31 | 2 |
| 621 | 133136 | R25377 | 4.60 | 45.45 | 9.89 | 4.65 | 0.98 | 24.03 | 2 |
| 622 | 266135 | N21633 | 0.39 | 3.82 | 9.87 | 1.10 | 17.62 | 10.88 | 2 |
| 623 | 154720 | R55220 | 0.01 | 0.09 | 9.86 | 8.07 | 19.71 | 1.81 | 2 |
| 624 | 812994 | AA464815 | 0.02 | 0.21 | 9.80 | 5.59 | 23.34 | 0.46 | 2 |
| 625 | 685801 | AA262080 | 0.64 | 6.27 | 9.80 | 2.04 | 22.72 | 4.63 | 2 |
| 626 | 38598 | R49126 | 0.28 | 2.73 | 9.79 | 1.50 | 24.64 | 3.25 | 2 |
| 627 | 490387 | AA120779 | 0.02 | 0.21 | 9.77 | 1.35 | 22.17 | 5.79 | 2 |
| 628 | 951305 | AA620528 | 0.62 | 5.97 | 9.68 | 1.31 | 9.16 | 18.56 | 2 |
| 629 | 782725 | AA447986 | 0.01 | 0.10 | 9.67 | 1.13 | 23.22 | 4.67 | 2 |
| 630 | 512116 | AA133590 | 0.64 | 6.12 | 9.62 | 0.66 | 21.91 | 6.28 | 2 |
| 631 | 841323 | AA487432 | 0.48 | 4.60 | 9.57 | 0.81 | 24.53 | 3.39 | 2 |
| 632 | 195695 | R89490 | 0.02 | 0.16 | 9.56 | 1.61 | 17.50 | 9.58 | 2 |
| 633 | 35804 | R46000 | 0.08 | 0.77 | 9.53 | 2.05 | 15.63 | 10.89 | 2 |
| 634 | 362926 | AA018980 | 0.70 | 6.70 | 9.51 | 24.42 | 3.74 | 0.37 | 2 |
| 635 | 375682 | AA032221 | 1.37 | 12.98 | 9.50 | 23.58 | 3.03 | 1.89 | 2 |
| 636 | 630013 | AA219080 | 0.15 | 1.40 | 9.48 | 1.15 | 11.85 | 15.44 | 2 |
| 637 | 320495 | W16659 | 0.01 | 0.09 | 9.47 | 10.45 | 1.00 | 16.97 | 2 |
| 638 | 866702 | AA679180 | 0.01 | 0.09 | 9.40 | 8.70 | 1.04 | 18.46 | 2 |
| 639 | 490959 | AA136664 | 0.53 | 5.02 | 9.39 | 1.84 | 5.32 | 21.00 | 2 |
| 640 | 48226 | H11519 | 0.41 | 3.84 | 9.37 | 0.66 | 23.58 | 3.86 | 2 |
| 641 | 435330 | AA699926 | 0.01 | 0.08 | 9.36 | 8.59 | 1.12 | 18.37 | 2 |
| 642 | 247628 | N54254 | 0.03 | 0.32 | 9.36 | 4.86 | 21.39 | 1.83 | 2 |
| 643 | 700792 | AA284072 | 0.01 | 0.06 | 9.34 | 11.43 | 1.54 | 15.06 | 2 |
| 644 | 281162 | N50962 | 0.02 | 0.22 | 9.31 | 0.97 | 12.53 | 14.44 | 2 |
| 645 | 204596 | H57060 | 0.01 | 0.09 | 9.30 | 4.26 | 1.86 | 21.78 | 2 |
| 646 | 487165 | AA045074 | 0.01 | 0.09 | 9.29 | 3.14 | 1.00 | 23.74 | 2 |
| 647 | 814791 | AA455242 | 0.68 | 6.35 | 9.29 | 1.35 | 16.72 | 9.79 | 2 |
| 648 | 1627705 | AI017607 | 0.44 | 4.10 | 9.26 | 2.30 | 7.70 | 17.77 | 2 |
| 649 | 230882 | R95962 | 0.10 | 0.87 | 9.18 | 2.37 | 19.73 | 5.45 | 2 |
| 650 | 199577 | R96579 | 0.27 | 2.44 | 9.12 | 0.81 | 13.04 | 13.51 | 2 |
| 651 | 320865 | W44766 | 0.03 | 0.26 | 9.07 | 12.50 | 0.36 | 14.35 | 2 |
| 652 | 490556 | AA100696 | 0.01 | 0.09 | 9.07 | 5.08 | 1.00 | 21.12 | 2 |
| 653 | 266259 | N26515 | 0.35 | 3.21 | 9.06 | 1.65 | 10.87 | 14.67 | 2 |
| 654 | 627541 | AA192527 | 0.40 | 3.62 | 9.05 | 1.54 | 20.24 | 5.38 | 2 |
| 655 | 877632 | AA488175 | 1.17 | 10.62 | 9.04 | 2.44 | 15.22 | 9.46 | 2 |
| 656 | 594758 | AA172056 | 1.30 | 11.71 | 9.03 | 12.54 | 13.45 | 1.10 | 2 |
| 657 | 591095 | AA158346 | 0.48 | 4.35 | 8.99 | 1.42 | 21.38 | 4.17 | 2 |
| 658 | 951241 | AA620485 | 1.98 | 17.81 | 8.98 | 10.05 | 0.30 | 16.60 | 2 |
| 659 | 730036 | AA416970 | 0.02 | 0.21 | 8.96 | 0.65 | 20.84 | 5.40 | 2 |
| 660 | 342640 | W68220 | 0.67 | 5.97 | 8.87 | 11.72 | 2.96 | 11.94 | 2 |
| 661 | 175533 | H41203 | 0.38 | 3.32 | 8.84 | 0.92 | 17.16 | 8.45 | 2 |
| 662 | 50227 | H16772 | 0.25 | 2.16 | 8.81 | 9.92 | 2.08 | 14.43 | 2 |
| 663 | 884511 | AA629999 | 4.51 | 39.21 | 8.70 | 0.36 | 17.44 | 8.30 | 2 |
| 664 | 1048995 | AA778646 | 0.09 | 0.80 | 8.69 | 1.34 | 6.64 | 18.10 | 2 |
| 665 | 345761 | W72671 | 0.53 | 4.61 | 8.69 | 3.59 | 21.88 | 0.61 | 2 |
| 666 | 743589 | AA609471 | 1.12 | 9.68 | 8.68 | 1.16 | 21.71 | 3.16 | 2 |
| 667 | 878544 | AA775863 | 3.96 | 34.28 | 8.65 | 1.87 | 16.58 | 7.50 | 2 |
| 668 | 28927 | R40373 | 0.59 | 5.05 | 8.60 | 0.60 | 21.85 | 3.36 | 2 |
| 669 | 897546 | AA496998 | 0.01 | 0.09 | 8.56 | 10.33 | 1.00 | 14.35 | 2 |
| 670 | 462926 | AA682321 | 0.60 | 5.11 | 8.53 | 6.22 | 0.52 | 18.86 | 2 |
| 671 | 300615 | N80764 | 0.83 | 6.98 | 8.42 | 0.78 | 6.50 | 17.99 | 2 |
| 672 | 115408 | T87515 | 1.40 | 11.80 | 8.41 | 1.96 | 3.76 | 19.50 | 2 |
| 673 | 324717 | W47364 | 1.13 | 9.51 | 8.39 | 1.45 | 12.46 | 11.27 | 2 |
| 674 | 825416 | AA504265 | 0.40 | 3.36 | 8.39 | 1.30 | 11.51 | 12.36 | 2 |
| 675 | 484535 | AA036974 | 0.01 | 0.08 | 8.39 | 1.81 | 6.16 | 17.21 | 2 |
| 676 | 566887 | AA132226 | 0.01 | 0.08 | 8.34 | 4.16 | 18.57 | 2.28 | 2 |
| 677 | 296429 | N74617 | 1.62 | 13.45 | 8.31 | 2.41 | 6.27 | 16.24 | 2 |
| 678 | 842968 | AA488324 | 1.30 | 10.75 | 8.30 | 5.22 | 0.41 | 19.27 | 2 |
| 679 | 37980 | R61372 | 0.13 | 1.09 | 8.29 | 8.74 | 13.18 | 2.96 | 2 |
| 680 | 280882 | N50806 | 0.02 | 0.15 | 8.25 | 9.98 | 1.67 | 13.09 | 2 |
| 681 | 380884 | AA058576 | 0.18 | 1.49 | 8.21 | 2.23 | 11.14 | 11.26 | 2 |
| 682 | 153355 | R47893 | 0.02 | 0.16 | 8.16 | 4.39 | 19.58 | 0.50 | 2 |
| 683 | 503581 | AA131239 | 0.02 | 0.14 | 8.15 | 5.83 | 0.56 | 18.07 | 2 |
| 684 | 784285 | AA447504 | 2.68 | 21.79 | 8.13 | 0.11 | 20.61 | 3.68 | 2 |
| 685 | 433253 | AA699427 | 0.76 | 6.11 | 8.08 | 1.22 | 13.54 | 9.48 | 2 |
| 686 | 435488 | AA701351 | 2.00 | 16.15 | 8.07 | 1.99 | 17.84 | 4.38 | 2 |
| 687 | 796117 | AA460961 | 0.01 | 0.09 | 8.04 | 3.70 | 2.94 | 17.47 | 2 |
| 688 | 838776 | AA457576 | 0.03 | 0.22 | 8.03 | 0.38 | 18.34 | 5.36 | 2 |
| 689 | 230261 | H94944 | 0.01 | 0.08 | 8.02 | 2.25 | 8.34 | 13.46 | 2 |
| 690 | 841620 | AA487674 | 0.71 | 5.71 | 8.00 | 0.81 | 4.66 | 18.54 | 2 |
| 691 | 193892 | H51765 | 1.10 | 8.81 | 8.00 | 0.92 | 14.68 | 8.40 | 2 |
| 692 | 701625 | AA284172 | 0.29 | 2.29 | 7.89 | 1.06 | 13.94 | 8.68 | 2 |
| 693 | 417920 | W90381 | 1.98 | 15.64 | 7.89 | 2.43 | 18.08 | 3.15 | 2 |
| 694 | 287598 | N62132 | 1.08 | 8.53 | 7.89 | 4.16 | 16.63 | 2.87 | 2 |
| 695 | 1420830 | AA826324 | 0.94 | 7.39 | 7.83 | 18.23 | 1.50 | 3.76 | 2 |
| 696 | 38833 | R50775 | 0.66 | 5.20 | 7.83 | 2.27 | 15.21 | 6.01 | 2 |
| 697 | 345090 | W74362 | 2.76 | 21.45 | 7.78 | 1.62 | 3.07 | 18.64 | 2 |
| 698 | 48330 | H14949 | 0.14 | 1.06 | 7.74 | 18.13 | 4.09 | 0.99 | 2 |
| 699 | 502444 | AA156795 | 0.02 | 0.14 | 7.73 | 0.57 | 7.43 | 15.20 | 2 |
| 700 | 788217 | AA453437 | 0.24 | 1.86 | 7.73 | 0.97 | 17.32 | 4.90 | 2 |
| 701 | 204644 | H57082 | 0.27 | 2.05 | 7.70 | 1.18 | 12.63 | 9.28 | 2 |
| 702 | 810661 | AA463982 | 0.01 | 0.08 | 7.69 | 7.96 | 14.11 | 1.00 | 2 |
| 703 | 435351 | AA700739 | 2.93 | 22.51 | 7.67 | 1.69 | 3.87 | 17.46 | 2 |
| 704 | 46927 | H10231 | 0.07 | 0.53 | 7.67 | 2.09 | 11.89 | 9.03 | 2 |
| 705 | 46740 | H10068 | 0.03 | 0.25 | 7.66 | 1.41 | 14.72 | 6.84 | 2 |
| 706 | 1035664 | AA780365 | 0.13 | 1.03 | 7.65 | 12.36 | 8.59 | 2.00 | 2 |
| 707 | 561971 | AA088886 | 3.04 | 23.26 | 7.64 | 2.69 | 16.52 | 3.72 | 2 |
| 708 | 345743 | W72666 | 2.36 | 18.08 | 7.64 | 1.23 | 16.02 | 5.66 | 2 |
| 709 | 120695 | T95804 | 0.01 | 0.08 | 7.61 | 0.83 | 12.70 | 9.32 | 2 |
| 710 | 725340 | AA291773 | 0.72 | 5.46 | 7.60 | 1.27 | 5.74 | 15.79 | 2 |
| 711 | 41789 | R59197 | 1.59 | 12.06 | 7.57 | 3.02 | 17.38 | 2.32 | 2 |
| 712 | 448032 | AA702802 | 0.01 | 0.08 | 7.54 | 5.64 | 1.00 | 15.99 | 2 |
| 713 | 39833 | R53455 | 0.97 | 7.28 | 7.53 | 0.35 | 12.36 | 9.88 | 2 |
| 714 | 868189 | AA633835 | 0.16 | 1.18 | 7.49 | 1.25 | 5.39 | 15.83 | 2 |
| 715 | 506018 | AA708441 | 0.70 | 5.21 | 7.48 | 3.13 | 18.13 | 1.19 | 2 |
| 716 | 811166 | AA485749 | 0.01 | 0.08 | 7.46 | 12.26 | 0.92 | 9.18 | 2 |
| 717 | 447167 | AA702973 | 0.33 | 2.44 | 7.45 | 1.82 | 15.92 | 4.60 | 2 |
| 718 | 128243 | R12473 | 0.83 | 6.14 | 7.44 | 1.30 | 8.97 | 12.04 | 2 |
| 719 | 37538 | R49644 | 0.04 | 0.31 | 7.41 | 0.99 | 11.96 | 9.29 | 2 |
| 720 | 826995 | AA521384 | 1.19 | 8.77 | 7.36 | 1.89 | 4.20 | 15.98 | 2 |
| 721 | 429519 | AA011390 | 0.60 | 4.44 | 7.35 | 1.85 | 16.45 | 3.75 | 2 |
| 722 | 278729 | N62936 | 1.35 | 9.91 | 7.34 | 0.34 | 13.66 | 8.02 | 2 |
| 723 | 814288 | AA459008 | 1.12 | 8.19 | 7.31 | 2.29 | 4.08 | 15.57 | 2 |
| 724 | 66317 | T66816 | 3.88 | 28.27 | 7.29 | 6.83 | 0.63 | 14.40 | 2 |
| 725 | 451907 | AA706968 | 2.08 | 15.11 | 7.27 | 11.07 | 0.08 | 10.67 | 2 |
| 726 | 31299 | R42946 | 0.52 | 3.80 | 7.27 | 4.52 | 16.05 | 1.23 | 2 |
| 727 | 266631 | N22766 | 0.05 | 0.35 | 7.24 | 1.57 | 9.92 | 10.22 | 2 |
| 728 | 32756 | R43308 | 0.30 | 2.20 | 7.23 | 7.23 | 2.06 | 12.41 | 2 |
| 729 | 50904 | H19234 | 0.16 | 1.17 | 7.23 | 1.81 | 13.06 | 6.82 | 2 |
| 730 | 565734 | AA135809 | 1.24 | 8.99 | 7.23 | 2.92 | 12.65 | 6.11 | 2 |
| 731 | 359722 | AA011182 | 0.04 | 0.31 | 7.22 | 1.78 | 13.46 | 6.43 | 2 |
| 732 | 809828 | AA455521 | 0.64 | 4.64 | 7.20 | 2.72 | 7.61 | 11.27 | 2 |
| 733 | 782503 | AA431773 | 1.57 | 11.27 | 7.19 | 2.94 | 3.28 | 15.36 | 2 |
| 734 | 504308 | AA131909 | 1.19 | 8.56 | 7.18 | 2.29 | 3.21 | 16.05 | 2 |
| 735 | 501868 | AA128005 | 0.03 | 0.24 | 7.18 | 2.42 | 4.52 | 14.61 | 2 |
| 736 | 825282 | AA504201 | 2.23 | 15.96 | 7.17 | 2.25 | 8.89 | 10.38 | 2 |
| 737 | 897271 | AA677655 | 0.02 | 0.11 | 7.14 | 0.87 | 8.75 | 11.80 | 2 |
| 738 | 1416782 | AA894557 | 14.77 | 105.24 | 7.13 | 13.51 | 0.03 | 7.84 | 2 |
| 739 | 814779 | AA455237 | 2.03 | 14.44 | 7.12 | 2.47 | 11.28 | 7.62 | 2 |
| 740 | 785845 | AA449118 | 0.09 | 0.62 | 7.11 | 13.68 | 2.34 | 5.32 | 2 |
| 741 | 199644 | R96520 | 0.21 | 1.51 | 7.11 | 1.14 | 11.35 | 8.84 | 2 |
| 742 | 812294 | AA455087 | 0.44 | 3.14 | 7.11 | 4.89 | 15.66 | 0.77 | 2 |
| 743 | 278531 | N66158 | 13.50 | 95.95 | 7.11 | 2.00 | 12.50 | 6.81 | 2 |
| 744 | 366778 | AA029722 | 0.04 | 0.26 | 7.05 | 3.04 | 17.83 | 0.27 | 2 |
| 745 | 898333 | AA598849 | 0.42 | 2.95 | 7.02 | 1.98 | 14.80 | 4.28 | 2 |
| 746 | 247050 | N53940 | 0.29 | 2.01 | 7.02 | 1.68 | 7.50 | 11.87 | 2 |
| 747 | 345330 | W72556 | 0.04 | 0.28 | 7.00 | 0.85 | 5.67 | 14.49 | 2 |
| 748 | 731139 | AA417288 | 0.25 | 1.75 | 6.98 | 0.86 | 16.93 | 3.14 | 2 |
| 749 | 82215 | T68878 | 0.10 | 0.68 | 6.95 | 2.37 | 9.22 | 9.27 | 2 |
| 750 | 811067 | AA485453 | 5.22 | 36.26 | 6.95 | 2.09 | 8.38 | 10.38 | 2 |
| 751 | 841221 | AA486741 | 0.01 | 0.05 | 6.94 | 3.97 | 1.38 | 15.45 | 2 |
| 752 | 79254 | T58146 | 0.01 | 0.07 | 6.93 | 14.50 | 5.14 | 1.18 | 2 |
| 753 | 1500162 | AA886739 | 0.55 | 3.78 | 6.90 | 2.34 | 9.72 | 8.65 | 2 |
| 754 | 856585 | AA669222 | 0.01 | 0.06 | 6.89 | 7.04 | 1.10 | 12.53 | 2 |
| 755 | 787938 | AA452278 | 0.40 | 2.74 | 6.88 | 10.95 | 1.51 | 8.19 | 2 |
| 756 | 79761 | T63980 | 3.48 | 23.86 | 6.86 | 4.74 | 2.38 | 13.46 | 2 |
| 757 | 586650 | AA129135 | 0.69 | 4.75 | 6.85 | 2.73 | 8.48 | 9.34 | 2 |
| 758 | 1641367 | AI025259 | 0.12 | 0.83 | 6.84 | 1.77 | 7.15 | 11.61 | 2 |
| 759 | 1556526 | AA935560 | 0.49 | 3.35 | 6.83 | 13.90 | 2.82 | 3.78 | 2 |
| 760 | 726791 | AA398406 | 0.04 | 0.25 | 6.83 | 1.52 | 13.88 | 5.09 | 2 |
| 761 | 204580 | H56903 | 0.41 | 2.79 | 6.80 | 1.22 | 12.71 | 6.48 | 2 |
| 762 | 131094 | R23246 | 0.69 | 4.70 | 6.80 | 1.08 | 3.80 | 15.52 | 2 |
| 763 | 813275 | AA455940 | 0.83 | 5.63 | 6.79 | 0.68 | 16.50 | 3.19 | 2 |
| 764 | 279397 | N48707 | 1.05 | 7.09 | 6.77 | 3.45 | 15.10 | 1.76 | 2 |
| 765 | 809503 | AA454562 | 0.24 | 1.65 | 6.77 | 12.08 | 7.25 | 0.98 | 2 |
| 766 | 811582 | AA454597 | 1.50 | 10.08 | 6.74 | 9.56 | 1.82 | 8.83 | 2 |
| 767 | 280154 | N47008 | 1.47 | 9.88 | 6.73 | 0.64 | 16.41 | 3.12 | 2 |
| 768 | 725677 | AA398949 | 1.11 | 7.44 | 6.71 | 2.08 | 10.73 | 7.33 | 2 |
| 769 | 43759 | H05085 | 0.58 | 3.87 | 6.71 | 3.11 | 14.81 | 2.21 | 2 |
| 770 | 1475028 | AA857413 | 62.96 | 421.08 | 6.69 | 1.87 | 4.25 | 13.94 | 2 |
| 771 | 40038 | R53446 | 0.51 | 3.42 | 6.68 | 1.33 | 3.97 | 14.72 | 2 |
| 772 | 321900 | W37447 | 0.59 | 3.96 | 6.67 | 1.35 | 14.02 | 4.64 | 2 |
| 773 | 1461316 | AA883391 | 0.45 | 3.02 | 6.67 | 2.21 | 13.65 | 4.14 | 2 |
| 774 | 593185 | AA165678 | 2.70 | 17.94 | 6.65 | 14.01 | 1.82 | 4.12 | 2 |
| 775 | 768172 | AA424790 | 1.17 | 7.75 | 6.64 | 11.27 | 0.58 | 8.09 | 2 |
| 776 | 781362 | AA448400 | 0.01 | 0.07 | 6.62 | 7.97 | 10.05 | 1.85 | 2 |
| 777 | 73638 | T55728 | 9.61 | 63.60 | 6.62 | 1.16 | 9.16 | 9.54 | 2 |
| 778 | 504396 | AA142869 | 0.40 | 2.64 | 6.58 | 2.28 | 12.93 | 4.54 | 2 |
| 779 | 470001 | AA029997 | 0.22 | 1.48 | 6.58 | 1.02 | 13.90 | 4.81 | 2 |
| 780 | 46916 | H09997 | 0.24 | 1.57 | 6.58 | 4.61 | 13.84 | 1.28 | 2 |
| 781 | 375827 | AA039851 | 0.02 | 0.12 | 6.57 | 2.44 | 7.59 | 9.68 | 2 |
| 782 | 72441 | T51617 | 0.04 | 0.24 | 6.51 | 1.47 | 11.33 | 6.74 | 2 |
| 783 | 300276 | N79745 | 0.81 | 5.27 | 6.51 | 1.45 | 4.25 | 13.83 | 2 |
| 784 | 32134 | R43328 | 1.01 | 6.55 | 6.50 | 2.35 | 11.59 | 5.55 | 2 |
| 785 | 1573251 | AA953357 | 1.34 | 8.71 | 6.49 | 10.79 | 2.65 | 6.04 | 2 |
| 786 | 898098 | AA598796 | 1.56 | 10.14 | 6.49 | 2.34 | 13.27 | 3.84 | 2 |
| 787 | 135688 | R32406 | 0.43 | 2.76 | 6.48 | 14.04 | 3.01 | 2.39 | 2 |
| 788 | 565493 | AA129677 | 0.01 | 0.07 | 6.47 | 4.90 | 1.84 | 12.67 | 2 |
| 789 | 773564 | AA428186 | 0.02 | 0.12 | 6.46 | 2.50 | 9.29 | 7.59 | 2 |
| 790 | 877784 | AA626788 | 4.30 | 27.63 | 6.43 | 1.38 | 9.13 | 8.78 | 2 |
| 791 | 788488 | AA452542 | 0.07 | 0.45 | 6.42 | 2.05 | 11.14 | 6.08 | 2 |
| 792 | 786078 | AA448664 | 1.05 | 6.74 | 6.41 | 2.85 | 6.70 | 9.70 | 2 |
| 793 | 159462 | H15910 | 0.03 | 0.21 | 6.41 | 5.69 | 12.25 | 1.28 | 2 |
| 794 | 194717 | R89846 | 0.26 | 1.66 | 6.39 | 0.90 | 6.06 | 12.22 | 2 |
| 795 | 268711 | N25936 | 1.55 | 9.86 | 6.37 | 13.36 | 1.59 | 4.18 | 2 |
| 796 | 277112 | N39611 | 0.59 | 3.76 | 6.37 | 2.87 | 7.88 | 8.37 | 2 |
| 797 | 810996 | AA485351 | 0.02 | 0.11 | 6.37 | 1.95 | 13.18 | 3.99 | 2 |
| 798 | 772410 | AA405533 | 0.71 | 4.54 | 6.37 | 1.54 | 5.53 | 12.04 | 2 |
| 799 | 196824 | R93068 | 0.24 | 1.50 | 6.36 | 0.76 | 12.20 | 6.13 | 2 |
| 800 | 814211 | AA465223 | 1.01 | 6.46 | 6.36 | 1.48 | 6.38 | 11.23 | 2 |
| 801 | 448417 | AA777555 | 0.53 | 3.38 | 6.36 | 13.51 | 4.43 | 1.13 | 2 |
| 802 | 85409 | T71991 | 4.22 | 26.80 | 6.35 | 2.86 | 4.38 | 11.81 | 2 |
| 803 | 814444 | AA459244 | 0.77 | 4.87 | 6.31 | 1.06 | 8.03 | 9.86 | 2 |
| 804 | 486538 | AA042812 | 8.25 | 51.96 | 6.30 | 1.79 | 10.78 | 6.32 | 2 |
| 805 | 416075 | W85878 | 0.93 | 5.87 | 6.29 | 2.64 | 8.05 | 8.17 | 2 |
| 806 | 289496 | N63988 | 0.35 | 2.21 | 6.28 | 1.45 | 10.13 | 7.28 | 2 |
| 807 | 302221 | N77828 | 0.22 | 1.37 | 6.27 | 7.40 | 2.46 | 8.95 | 2 |
| 808 | 203179 | H54659 | 0.25 | 1.58 | 6.27 | 2.11 | 8.82 | 7.88 | 2 |
| 809 | 34254 | R44201 | 0.02 | 0.15 | 6.27 | 2.51 | 6.21 | 10.08 | 2 |
| 810 | 287135 | N24869 | 0.37 | 2.29 | 6.25 | 0.27 | 5.01 | 13.49 | 2 |
| 811 | 769857 | AA430367 | 2.19 | 13.69 | 6.25 | 13.18 | 0.27 | 5.30 | 2 |
| 812 | 1475308 | AA877997 | 0.80 | 4.98 | 6.24 | 13.52 | 3.53 | 1.69 | 2 |
| 813 | 435957 | AA701978 | 0.66 | 4.11 | 6.23 | 0.87 | 12.34 | 5.48 | 2 |
| 814 | 23073 | R38539 | 0.22 | 1.36 | 6.22 | 0.68 | 8.92 | 9.06 | 2 |
| 815 | 52128 | H22568 | 1.94 | 12.01 | 6.20 | 1.35 | 7.60 | 9.66 | 2 |
| 816 | 854897 | AA630373 | 0.64 | 3.99 | 6.20 | 2.28 | 12.42 | 3.89 | 2 |
| 817 | 320602 | W31389 | 0.01 | 0.08 | 6.20 | 11.05 | 0.76 | 6.77 | 2 |
| 818 | 461933 | AA779949 | 1.43 | 8.84 | 6.19 | 12.11 | 3.72 | 2.74 | 2 |
| 819 | 277476 | N56875 | 0.05 | 0.33 | 6.16 | 1.20 | 10.69 | 6.60 | 2 |
| 820 | 1470195 | AA865924 | 0.42 | 2.57 | 6.15 | 8.48 | 2.18 | 7.78 | 2 |
| 821 | 854701 | AA630104 | 2.60 | 15.94 | 6.13 | 0.57 | 7.55 | 10.29 | 2 |
| 822 | 770992 | AA430744 | 2.03 | 12.43 | 6.13 | 6.15 | 2.69 | 9.56 | 2 |
| 823 | 1032080 | AA809891 | 0.47 | 2.89 | 6.13 | 1.59 | 7.78 | 9.04 | 2 |
| 824 | 950945 | AA608752 | 1.63 | 10.01 | 6.12 | 1.47 | 13.05 | 3.85 | 2 |
| 825 | 133192 | R26456 | 0.02 | 0.15 | 6.12 | 4.68 | 12.18 | 1.49 | 2 |
| 826 | 195817 | R92186 | 0.39 | 2.40 | 6.11 | 1.12 | 9.73 | 7.48 | 2 |
| 827 | 781109 | AA430052 | 2.62 | 16.02 | 6.11 | 2.39 | 8.86 | 7.07 | 2 |
| 828 | 753862 | AA410517 | 34.08 | 207.98 | 6.10 | 2.76 | 3.64 | 11.91 | 2 |
| 829 | 814303 | AA459106 | 4.36 | 26.54 | 6.09 | 1.52 | 11.73 | 5.03 | 2 |
| 830 | 33342 | R44617 | 0.01 | 0.06 | 6.06 | 13.72 | 3.46 | 1.00 | 2 |
| 831 | 378016 | AA775557 | 0.12 | 0.70 | 6.03 | 1.70 | 4.07 | 12.34 | 2 |
| 832 | 594693 | AA165313 | 0.88 | 5.33 | 6.03 | 4.85 | 0.62 | 12.62 | 2 |
| 833 | 788225 | AA453441 | 2.71 | 16.34 | 6.02 | 1.83 | 11.45 | 4.79 | 2 |
| 834 | 38027 | R59369 | 0.07 | 0.39 | 6.02 | 2.14 | 11.73 | 4.18 | 2 |
| 835 | 813410 | AA458646 | 1.29 | 7.71 | 6.00 | 2.89 | 9.92 | 5.20 | 2 |
| 836 | 1292190 | AA705840 | 0.09 | 0.54 | 5.99 | 4.63 | 10.65 | 2.68 | 2 |
| 837 | 814080 | AA465353 | 1.48 | 8.89 | 5.99 | 0.97 | 9.75 | 7.25 | 2 |
| 838 | 825284 | AA504202 | 1.39 | 8.34 | 5.98 | 2.44 | 7.62 | 7.87 | 2 |
| 839 | 276972 | N39233 | 0.50 | 2.99 | 5.98 | 1.57 | 8.57 | 7.79 | 2 |
| 840 | 49204 | H15696 | 1.28 | 7.64 | 5.98 | 2.42 | 11.00 | 4.51 | 2 |
| 841 | 45607 | H08210 | 0.08 | 0.49 | 5.96 | 2.48 | 3.88 | 11.52 | 2 |
| 842 | 1505666 | AA879423 | 0.09 | 0.55 | 5.96 | 2.30 | 9.04 | 6.52 | 2 |
| 843 | 563860 | AA101173 | 0.12 | 0.69 | 5.93 | 0.97 | 11.46 | 5.38 | 2 |
| 844 | 289645 | N62866 | 0.31 | 1.85 | 5.93 | 11.41 | 0.30 | 6.09 | 2 |
| 845 | 272888 | N36008 | 2.68 | 15.84 | 5.91 | 1.63 | 7.34 | 8.76 | 2 |
| 846 | 796366 | AA456135 | 0.91 | 5.37 | 5.91 | 13.24 | 3.44 | 1.04 | 2 |
| 847 | 284783 | N59866 | 0.06 | 0.36 | 5.90 | 6.66 | 8.19 | 2.86 | 2 |
| 848 | 771165 | AA429474 | 1.11 | 6.55 | 5.89 | 2.20 | 11.44 | 4.05 | 2 |
| 849 | 1048949 | AA778603 | 0.13 | 0.76 | 5.88 | 0.84 | 8.58 | 8.22 | 2 |
| 850 | 246649 | N73252 | 0.43 | 2.56 | 5.88 | 1.87 | 6.81 | 8.94 | 2 |
| 851 | 773367 | AA425684 | 0.05 | 0.30 | 5.85 | 1.25 | 12.13 | 4.16 | 2 |
| 852 | 429376 | AA007529 | 0.29 | 1.67 | 5.84 | 10.90 | 2.57 | 4.04 | 2 |
| 853 | 1343468 | AA709271 | 0.69 | 4.03 | 5.83 | 8.54 | 2.32 | 6.64 | 2 |
| 854 | 488516 | AA044565 | 0.07 | 0.41 | 5.83 | 3.22 | 12.15 | 2.11 | 2 |
| 855 | 150623 | H02158 | 0.01 | 0.06 | 5.82 | 6.78 | 1.00 | 9.69 | 2 |
| 856 | 506032 | AA708446 | 1.32 | 7.68 | 5.80 | 2.25 | 3.75 | 11.41 | 2 |
| 857 | 814995 | AA485090 | 0.83 | 4.83 | 5.80 | 2.24 | 9.55 | 5.62 | 2 |
| 858 | 287349 | N69068 | 0.75 | 4.34 | 5.79 | 1.15 | 12.93 | 3.29 | 2 |
| 859 | 429352 | AA007516 | 0.69 | 4.01 | 5.79 | 3.05 | 13.23 | 1.09 | 2 |
| 860 | 430336 | AA010619 | 0.03 | 0.14 | 5.79 | 0.30 | 4.68 | 12.39 | 2 |
| 861 | 1031113 | AA609914 | 0.04 | 0.22 | 5.79 | 1.21 | 10.89 | 5.26 | 2 |
| 862 | 506483 | AA708605 | 0.10 | 0.59 | 5.76 | 1.96 | 10.22 | 5.11 | 2 |
| 863 | 36480 | R46794 | 0.35 | 2.01 | 5.76 | 1.36 | 11.11 | 4.82 | 2 |
| 864 | 287581 | N62128 | 0.82 | 4.73 | 5.76 | 5.27 | 10.45 | 1.55 | 2 |
| 865 | 26505 | R20639 | 0.15 | 0.84 | 5.76 | 4.39 | 1.25 | 11.63 | 2 |
| 866 | 28475 | R13434 | 0.48 | 2.62 | 5.74 | 1.10 | 7.72 | 8.40 | 2 |
| 867 | 38510 | R50752 | 0.59 | 3.37 | 5.74 | 2.46 | 7.30 | 7.45 | 2 |
| 868 | 46862 | H10788 | 1.07 | 6.12 | 5.73 | 2.42 | 9.02 | 5.77 | 2 |
| 869 | 191546 | H37817 | 0.24 | 1.35 | 5.72 | 0.89 | 6.27 | 10.01 | 2 |
| 870 | 42842 | R60223 | 0.33 | 1.90 | 5.71 | 1.62 | 12.44 | 3.07 | 2 |
| 871 | 27817 | R40481 | 0.03 | 0.15 | 5.71 | 2.14 | 9.62 | 5.36 | 2 |
| 872 | 34063 | R45952 | 0.28 | 1.58 | 5.68 | 5.72 | 9.78 | 1.55 | 2 |
| 873 | 291374 | N72274 | 1.00 | 5.71 | 5.68 | 1.00 | 6.51 | 9.54 | 2 |
| 874 | 282104 | N51498 | 0.51 | 2.92 | 5.68 | 5.83 | 9.35 | 1.87 | 2 |
| 875 | 503865 | AA131707 | 1.39 | 7.88 | 5.68 | 1.17 | 12.71 | 3.16 | 2 |
| 876 | 196037 | R89363 | 0.41 | 2.35 | 5.68 | 1.17 | 8.12 | 7.73 | 2 |
| 877 | 878600 | AA775259 | 1.27 | 7.19 | 5.67 | 1.02 | 4.30 | 11.69 | 2 |
| 878 | 429517 | AA011389 | 0.01 | 0.06 | 5.67 | 3.76 | 1.00 | 12.25 | 2 |
| 879 | 39219 | R51382 | 0.14 | 0.81 | 5.66 | 3.85 | 12.66 | 0.46 | 2 |
| 880 | 433474 | AA699580 | 3.28 | 18.53 | 5.65 | 8.40 | 2.36 | 6.19 | 2 |
| 881 | 856592 | AA669218 | 1.19 | 6.68 | 5.64 | 10.55 | 1.37 | 5.00 | 2 |
| 882 | 395609 | AA757588 | 0.05 | 0.25 | 5.62 | 0.55 | 7.77 | 8.55 | 2 |
| 883 | 770979 | AA427400 | 0.02 | 0.10 | 5.62 | 5.02 | 0.44 | 11.39 | 2 |
| 884 | 357344 | W93500 | 0.01 | 0.06 | 5.62 | 5.85 | 8.42 | 2.59 | 2 |
| 885 | 869375 | AA679907 | 0.03 | 0.18 | 5.61 | 1.75 | 11.49 | 3.58 | 2 |
| 886 | 950926 | AA608730 | 3.17 | 17.75 | 5.60 | 8.06 | 1.26 | 7.48 | 2 |
| 887 | 361688 | W96197 | 0.01 | 0.07 | 5.60 | 0.80 | 10.00 | 5.99 | 2 |
| 888 | 193139 | H47315 | 0.03 | 0.17 | 5.59 | 1.43 | 9.88 | 5.46 | 2 |
| 889 | 121798 | T98201 | 0.72 | 4.03 | 5.59 | 6.89 | 7.41 | 2.47 | 2 |
| 890 | 789049 | AA452909 | 1.97 | 11.01 | 5.58 | 1.16 | 11.14 | 4.42 | 2 |
| 891 | 43977 | H05563 | 0.70 | 3.89 | 5.55 | 11.02 | 1.27 | 4.36 | 2 |
| 892 | 471707 | AA035580 | 0.66 | 3.65 | 5.55 | 6.12 | 2.85 | 7.67 | 2 |
| 893 | 783987 | AA443288 | 0.01 | 0.05 | 5.53 | 3.23 | 12.83 | 0.55 | 2 |
| 894 | 878798 | AA670408 | 23.98 | 132.57 | 5.53 | 1.02 | 7.04 | 8.52 | 2 |
| 895 | 1505898 | AA907714 | 0.07 | 0.41 | 5.52 | 2.10 | 8.01 | 6.45 | 2 |
| 896 | 130858 | R22206 | 0.99 | 5.42 | 5.50 | 2.27 | 10.54 | 3.69 | 2 |
| 897 | 839081 | AA487608 | 12.19 | 66.94 | 5.49 | 7.57 | 0.09 | 8.81 | 2 |
| 898 | 130845 | R22308 | 2.02 | 11.11 | 5.49 | 0.87 | 7.74 | 7.85 | 2 |
| 899 | 241432 | H80685 | 1.63 | 8.93 | 5.48 | 7.11 | 7.64 | 1.70 | 2 |
| 900 | 179556 | H51425 | 0.01 | 0.05 | 5.48 | 3.34 | 1.00 | 12.10 | 2 |
| 901 | 197206 | R92812 | 0.98 | 5.38 | 5.47 | 0.67 | 8.14 | 7.61 | 2 |
| 902 | 825408 | AA504262 | 1.29 | 7.03 | 5.47 | 1.85 | 10.90 | 3.66 | 2 |
| 903 | 525799 | AA074446 | 0.41 | 2.26 | 5.46 | 4.45 | 1.50 | 10.42 | 2 |
| 904 | 773170 | AA428514 | 0.05 | 0.26 | 5.45 | 1.75 | 8.29 | 6.30 | 2 |
| 905 | 256720 | H96356 | 0.01 | 0.05 | 5.44 | 1.86 | 6.97 | 7.49 | 2 |
| 906 | 195786 | R89287 | 0.19 | 1.05 | 5.44 | 1.09 | 6.79 | 8.44 | 2 |
| 907 | 1466998 | AA884428 | 0.08 | 0.45 | 5.43 | 2.62 | 8.15 | 5.51 | 2 |
| 908 | 451805 | AA706829 | 6.18 | 33.51 | 5.42 | 1.61 | 5.96 | 8.69 | 2 |
| 909 | 1487751 | AA883100 | 0.11 | 0.59 | 5.41 | 1.84 | 6.69 | 7.71 | 2 |
| 910 | 1580342 | AA969184 | 0.18 | 1.00 | 5.40 | 1.10 | 3.31 | 11.81 | 2 |
| 911 | 85561 | T73031 | 0.02 | 0.12 | 5.40 | 2.12 | 10.54 | 3.53 | 2 |
| 912 | 50939 | H18630 | 0.51 | 2.76 | 5.39 | 1.04 | 10.79 | 4.35 | 2 |
| 913 | 814360 | AA458827 | 1.32 | 7.13 | 5.38 | 2.06 | 5.32 | 8.77 | 2 |
| 914 | 823566 | AA497034 | 2.10 | 11.29 | 5.38 | 1.01 | 10.96 | 4.15 | 2 |
| 915 | 34462 | R44292 | 0.01 | 0.05 | 5.38 | 4.05 | 1.00 | 11.07 | 2 |
| 916 | 741790 | AA402965 | 2.17 | 11.61 | 5.35 | 1.13 | 6.68 | 8.24 | 2 |
| 917 | 345077 | W72310 | 0.05 | 0.28 | 5.33 | 1.34 | 10.44 | 4.21 | 2 |
| 918 | 156473 | R73525 | 0.01 | 0.05 | 5.32 | 8.91 | 1.01 | 6.03 | 2 |
| 919 | 129664 | R16676 | 1.18 | 6.28 | 5.31 | 1.15 | 7.63 | 7.16 | 2 |
| 920 | 898258 | AA598668 | 0.40 | 2.11 | 5.31 | 1.02 | 7.20 | 7.71 | 2 |
| 921 | 878550 | AA775865 | 2.76 | 14.65 | 5.30 | 2.14 | 3.73 | 10.03 | 2 |
| 922 | 813997 | AA455652 | 4.09 | 21.66 | 5.30 | 0.94 | 5.86 | 9.09 | 2 |
| 923 | 265102 | N21334 | 0.76 | 4.03 | 5.29 | 1.07 | 9.61 | 5.20 | 2 |
| 924 | 247089 | N57858 | 0.04 | 0.23 | 5.29 | 1.93 | 6.76 | 7.18 | 2 |
| 925 | 108395 | T77840 | 3.81 | 20.12 | 5.29 | 2.59 | 8.00 | 5.27 | 2 |
| 926 | 1055831 | AA628190 | 0.08 | 0.41 | 5.28 | 2.22 | 6.68 | 6.95 | 2 |
| 927 | 345787 | W72679 | 0.85 | 4.49 | 5.27 | 4.81 | 0.18 | 10.80 | 2 |
| 928 | 204299 | H59305 | 0.70 | 3.68 | 5.26 | 5.35 | 2.95 | 7.50 | 2 |
| 929 | 1505735 | AA879474 | 0.03 | 0.14 | 5.26 | 5.79 | 2.97 | 7.02 | 2 |
| 930 | 264646 | N20338 | 0.03 | 0.15 | 5.26 | 0.36 | 4.37 | 11.04 | 2 |
| 931 | 1048685 | AA620614 | 0.11 | 0.58 | 5.24 | 2.21 | 5.07 | 8.43 | 2 |
| 932 | 490753 | AA133166 | 8.60 | 44.95 | 5.23 | 1.93 | 7.32 | 6.43 | 2 |
| 933 | 594517 | AA169645 | 2.92 | 15.25 | 5.23 | 2.51 | 5.49 | 7.68 | 2 |
| 934 | 758360 | AA404290 | 0.02 | 0.08 | 5.22 | 4.20 | 10.92 | 0.55 | 2 |
| 935 | 324665 | AA284280 | 0.36 | 1.87 | 5.21 | 3.19 | 10.57 | 1.88 | 2 |
| 936 | 321386 | W32272 | 0.15 | 0.76 | 5.20 | 10.06 | 1.80 | 3.74 | 2 |
| 937 | 489881 | AA121504 | 0.09 | 0.46 | 5.20 | 3.27 | 10.71 | 1.61 | 2 |
| 938 | 784126 | AA446748 | 0.07 | 0.35 | 5.19 | 2.95 | 5.99 | 8.84 | 2 |
| 939 | 845602 | AA644334 | 1.83 | 9.47 | 5.18 | 1.72 | 6.91 | 6.90 | 2 |
| 940 | 768146 | AA426561 | 0.10 | 0.51 | 5.17 | 1.23 | 9.14 | 5.15 | 2 |
| 941 | 726658 | AA398218 | 0.01 | 0.05 | 5.15 | 7.12 | 1.00 | 7.33 | 2 |
| 942 | 501934 | AA129931 | 4.32 | 22.27 | 5.15 | 4.21 | 1.15 | 10.09 | 2 |
| 943 | 415806 | W84774 | 0.01 | 0.06 | 5.15 | 6.70 | 0.80 | 7.94 | 2 |
| 944 | 263342 | H99997 | 1.94 | 9.93 | 5.12 | 1.82 | 3.54 | 10.01 | 2 |
| 945 | 23658 | R38169 | 0.01 | 0.07 | 5.11 | 0.72 | 11.06 | 3.56 | 2 |
| 946 | 1293016 | AA683338 | 0.04 | 0.19 | 5.11 | 1.68 | 5.30 | 8.34 | 2 |
| 947 | 448920 | AA777779 | 0.08 | 0.43 | 5.10 | 1.62 | 8.40 | 5.28 | 2 |
| 948 | 491524 | AA148505 | 6.40 | 32.60 | 5.09 | 1.14 | 9.02 | 5.12 | 2 |
| 949 | 155575 | R71689 | 0.39 | 1.98 | 5.09 | 2.35 | 5.29 | 7.63 | 2 |
| 950 | 1602209 | AA962541 | 3.26 | 16.55 | 5.07 | 6.08 | 8.82 | 0.32 | 2 |
| 951 | 739247 | AA421230 | 8.07 | 40.76 | 5.05 | 1.54 | 8.12 | 5.48 | 2 |
| 952 | 824109 | AA490805 | 0.04 | 0.22 | 5.04 | 0.82 | 8.65 | 5.67 | 2 |
| 953 | 243653 | N49899 | 0.69 | 3.45 | 5.02 | 1.63 | 5.51 | 7.92 | 2 |
| 954 | 206052 | H61552 | 0.91 | 4.54 | 4.99 | 0.91 | 8.28 | 5.79 | 2 |
| 955 | 504279 | AA149637 | 1.71 | 8.53 | 4.99 | 7.00 | 6.10 | 1.87 | 2 |
| 956 | 825603 | AA504631 | 5.33 | 26.53 | 4.97 | 1.25 | 10.25 | 3.42 | 2 |
| 957 | 254162 | N20480 | 0.39 | 1.93 | 4.97 | 1.99 | 6.33 | 6.61 | 2 |
| 958 | 34294 | R44346 | 1.28 | 6.34 | 4.95 | 0.98 | 8.07 | 5.79 | 2 |
| 959 | 186132 | H39560 | 0.01 | 0.05 | 4.94 | 8.10 | 5.74 | 0.97 | 2 |
| 960 | 784772 | AA478542 | 1.78 | 8.75 | 4.92 | 0.97 | 8.14 | 5.65 | 2 |
| 961 | 147925 | R82041 | 0.06 | 0.30 | 4.91 | 1.91 | 6.08 | 6.75 | 2 |
| 962 | 277545 | N56982 | 0.43 | 2.11 | 4.90 | 1.20 | 6.72 | 6.79 | 2 |
| 963 | 563201 | AA114106 | 0.02 | 0.08 | 4.90 | 5.15 | 7.27 | 2.27 | 2 |
| 964 | 50114 | H16743 | 1.70 | 8.32 | 4.90 | 0.37 | 7.99 | 6.33 | 2 |
| 965 | 1504152 | AA904969 | 0.14 | 0.71 | 4.89 | 1.97 | 6.45 | 6.25 | 2 |
| 966 | 40721 | R55750 | 0.30 | 1.49 | 4.88 | 1.57 | 7.62 | 5.46 | 2 |
| 967 | 471598 | AA035384 | 7.11 | 34.71 | 4.88 | 2.09 | 5.39 | 7.16 | 2 |
| 968 | 486787 | AA043228 | 5.90 | 28.69 | 4.87 | 0.13 | 3.68 | 10.79 | 2 |
| 969 | 290560 | N62372 | 2.16 | 10.43 | 4.84 | 2.45 | 5.47 | 6.59 | 2 |
| 970 | 809508 | AA454554 | 0.01 | 0.05 | 4.83 | 8.44 | 5.04 | 1.00 | 2 |
| 971 | 382451 | AA064627 | 2.91 | 13.99 | 4.80 | 0.71 | 8.39 | 5.32 | 2 |
| 972 | 32517 | R43271 | 0.01 | 0.05 | 4.80 | 7.91 | 5.50 | 1.00 | 2 |
| 973 | 838568 | AA456931 | 19.35 | 92.90 | 4.80 | 2.12 | 5.68 | 6.60 | 2 |
| 974 | 258966 | N31641 | 1.57 | 7.55 | 4.80 | 1.94 | 5.26 | 7.20 | 2 |
| 975 | 503715 | AA131526 | 5.14 | 24.62 | 4.79 | 2.51 | 6.24 | 5.62 | 2 |
| 976 | 156048 | R72434 | 0.02 | 0.10 | 4.76 | 3.62 | 10.08 | 0.65 | 2 |
| 977 | 154472 | R54846 | 0.72 | 3.44 | 4.77 | 1.60 | 6.79 | 5.93 | 2 |
| 978 | 204129 | H55915 | 0.35 | 1.68 | 4.75 | 1.18 | 6.82 | 6.25 | 2 |
| 979 | 826089 | AA521411 | 0.13 | 0.60 | 4.75 | 1.30 | 6.47 | 6.47 | 2 |
| 980 | 123255 | R00275 | 0.92 | 4.36 | 4.75 | 2.49 | 5.38 | 6.37 | 2 |
| 981 | 453005 | AA779165 | 3.47 | 16.44 | 4.74 | 5.66 | 0.08 | 8.48 | 2 |
| 982 | 460553 | AA700415 | 0.06 | 0.31 | 4.74 | 2.27 | 5.74 | 6.21 | 2 |
| 983 | 43826 | H05766 | 0.96 | 4.54 | 4.72 | 2.52 | 5.51 | 6.12 | 2 |
| 984 | 432732 | AA701587 | 0.03 | 0.12 | 4.67 | 6.88 | 1.33 | 5.80 | 2 |
| 985 | 882488 | AA676590 | 0.06 | 0.27 | 4.65 | 6.62 | 5.90 | 1.44 | 2 |
| 986 | 46097 | H08796 | 0.39 | 1.82 | 4.65 | 5.94 | 2.63 | 5.38 | 2 |
| 987 | 809466 | AA443094 | 0.90 | 4.18 | 4.63 | 2.19 | 5.33 | 6.39 | 2 |
| 988 | 745192 | AA626847 | 1.12 | 5.17 | 4.62 | 2.36 | 5.63 | 5.88 | 2 |
| 989 | 898083 | AA598797 | 3.37 | 15.49 | 4.59 | 2.32 | 5.20 | 6.25 | 2 |
| 990 | 796606 | AA460521 | 0.97 | 4.45 | 4.59 | 2.55 | 5.24 | 5.98 | 2 |
| 991 | 327245 | AA284291 | 0.98 | 4.50 | 4.59 | 2.99 | 5.72 | 5.05 | 2 |
| 992 | 744044 | AA629251 | 0.01 | 0.06 | 4.58 | 5.99 | 0.75 | 7.00 | 2 |
| 993 | 882355 | AA676296 | 2.64 | 12.03 | 4.56 | 6.34 | 5.06 | 2.28 | 2 |
| 994 | 261567 | H98655 | 2.99 | 13.59 | 4.54 | 1.15 | 6.62 | 5.85 | 2 |
| 995 | 824728 | AA488979 | 1.11 | 5.05 | 4.54 | 0.93 | 7.47 | 5.23 | 2 |
| 996 | 283208 | N51367 | 0.59 | 2.67 | 4.54 | 0.99 | 7.39 | 5.23 | 2 |
| 997 | 453229 | AA704858 | 0.08 | 0.38 | 4.54 | 1.08 | 5.53 | 7.00 | 2 |
| 998 | 132954 | R24451 | 0.65 | 2.96 | 4.53 | 2.21 | 5.84 | 5.54 | 2 |
| 999 | 731031 | AA421270 | 8.95 | 40.40 | 4.52 | 2.12 | 6.30 | 5.12 | 2 |
| 1000 | 1468466 | AA885096 | 0.60 | 2.71 | 4.50 | 5.38 | 2.55 | 5.56 | 2 |
| 1001 | 206272 | H58542 | 1.42 | 6.39 | 4.50 | 1.27 | 5.28 | 6.95 | 2 |
| 1002 | 75650 | T58434 | 1.01 | 4.53 | 4.49 | 2.26 | 6.17 | 5.02 | 2 |
| 1003 | 257312 | N29624 | 0.36 | 1.60 | 4.48 | 0.90 | 6.98 | 5.57 | 2 |
| 1004 | 1048899 | AA778562 | 0.07 | 0.32 | 4.48 | 1.56 | 5.09 | 6.78 | 2 |
| 1005 | 51700 | H22854 | 0.24 | 1.09 | 4.47 | 6.45 | 5.25 | 1.71 | 2 |
| 1006 | 1466599 | AA883660 | 0.12 | 0.53 | 4.46 | 1.21 | 6.24 | 5.93 | 2 |
| 1007 | 51328 | H20743 | 0.01 | 0.06 | 4.44 | 2.31 | 5.02 | 5.98 | 2 |
| 1008 | 1504005 | AA904776 | 0.11 | 0.49 | 4.44 | 1.72 | 5.71 | 5.87 | 2 |
| 1009 | 194811 | R89765 | 0.42 | 1.88 | 4.42 | 0.87 | 5.83 | 6.76 | 2 |
| 1010 | 126239 | R06372 | 0.02 | 0.08 | 4.42 | 5.24 | 2.84 | 5.17 | 2 |
| 1011 | 726858 | AA398355 | 1.71 | 7.57 | 4.42 | 7.74 | 0.22 | 5.29 | 2 |
| 1012 | 110772 | T90621 | 13.07 | 57.53 | 4.40 | 2.19 | 5.60 | 5.41 | 2 |
| 1013 | 758302 | AA404338 | 0.03 | 0.14 | 4.39 | 6.57 | 0.31 | 6.30 | 2 |
| 1014 | 530545 | AA112979 | 0.19 | 0.84 | 4.38 | 2.44 | 5.46 | 5.23 | 2 |
| 1015 | 1461715 | AA884382 | 0.17 | 0.73 | 4.35 | 2.38 | 5.14 | 5.54 | 2 |
| 1016 | 745523 | AA626247 | 0.09 | 0.40 | 4.35 | 1.74 | 6.04 | 5.26 | 2 |
| 1017 | 744911 | AA625791 | 0.10 | 0.42 | 4.34 | 1.92 | 5.25 | 5.86 | 2 |
| 1018 | 784589 | AA443300 | 0.02 | 0.10 | 4.33 | 5.57 | 6.96 | 0.44 | 2 |
| 1019 | 647816 | AA205320 | 0.25 | 1.07 | 4.32 | 0.99 | 5.51 | 6.47 | 2 |
| 1020 | 80946 | T70122 | 5.27 | 22.74 | 4.32 | 1.50 | 5.60 | 5.84 | 2 |
| 1021 | 757463 | AA437245 | 0.09 | 0.39 | 4.28 | 1.45 | 5.78 | 5.62 | 2 |
| 1022 | 45514 | H08227 | 0.06 | 0.27 | 4.28 | 1.28 | 5.77 | 5.79 | 2 |
| 1023 | 280909 | N50828 | 1.43 | 6.07 | 4.25 | 1.08 | 5.61 | 6.07 | 2 |
| 1024 | 814906 | AA465692 | 2.68 | 11.37 | 4.24 | 1.91 | 5.44 | 5.36 | 2 |
| 1025 | 272238 | N35579 | 2.10 | 8.82 | 4.19 | 1.25 | 5.55 | 5.76 | 2 |
| 1026 | 42400 | R60949 | 1.37 | 5.73 | 4.17 | 5.58 | 1.07 | 5.87 | 2 |
| 1027 | 281103 | N50935 | 2.75 | 11.43 | 4.16 | 1.40 | 5.50 | 5.58 | 2 |
| 1028 | 1466824 | AA883755 | 0.09 | 0.36 | 4.16 | 1.38 | 5.73 | 5.36 | 2 |
| 1029 | 505997 | AA708431 | 0.41 | 1.69 | 4.14 | 5.38 | 1.63 | 5.41 | 2 |
| 1030 | 841287 | AA487206 | 2.36 | 9.76 | 4.13 | 0.84 | 6.05 | 5.49 | 2 |
| 1031 | 126695 | R07012 | 0.14 | 0.57 | 4.12 | 1.58 | 5.14 | 5.65 | 2 |
| 1032 | 858292 | AA633993 | 2.38 | 9.78 | 4.10 | 0.57 | 5.73 | 6.01 | 2 |
| 1033 | 1469211 | AA862814 | 0.20 | 0.83 | 4.09 | 1.23 | 5.12 | 5.93 | 2 |
| 1034 | 73659 | T54527 | 1.12 | 4.55 | 4.08 | 0.97 | 5.71 | 5.55 | 2 |
| 1035 | 79726 | T82552 | 1.04 | 4.25 | 4.08 | 5.88 | 5.23 | 1.12 | 2 |
| 1036 | 292388 | N79230 | 1.82 | 7.30 | 4.01 | 5.25 | 1.78 | 5.01 | 2 |
| 1037 | 239661 | H79566 | 0.02 | 0.07 | 3.92 | 5.37 | 5.82 | 0.56 | 2 |
| 1038 | 882483 | AA676598 | 2.77 | 10.81 | 3.91 | 1.13 | 5.04 | 5.56 | 2 |
| 1039 | 249517 | H84915 | 0.76 | 2.97 | 3.90 | 1.12 | 5.12 | 5.46 | 2 |
| 1040 | 247587 | N58136 | 0.02 | 0.09 | 3.90 | 5.46 | 5.69 | 0.55 | 2 |
| 1041 | 193420 | H47089 | 0.40 | 1.56 | 3.87 | 1.01 | 5.60 | 5.01 | 2 |
| 1042 | 487141 | AA045340 | 1.95 | 7.56 | 3.87 | 0.43 | 5.56 | 5.63 | 2 |
| 1043 | 260168 | N32071 | 0.01 | 12.21 | 1221.36 | 0.55 | 3660.91 | 2.63 | 1 |
| 1044 | 280291 | N47961 | 0.01 | 5.59 | 559.28 | 1.63 | 1675.21 | 1.00 | 1 |
| 1045 | 307328 | N93470 | 0.02 | 7.11 | 421.73 | 1.71 | 1262.88 | 0.59 | 1 |
| 1046 | 279613 | N48913 | 0.39 | 158.35 | 411.27 | 0.98 | 1231.52 | 1.33 | 1 |
| 1047 | 268115 | N30131 | 0.01 | 2.55 | 254.59 | 1.49 | 761.20 | 1.09 | 1 |
| 1048 | 124567 | R01937 | 0.01 | 2.45 | 244.61 | 2.76 | 730.07 | 1.00 | 1 |
| 1049 | 327495 | W20486 | 0.01 | 2.38 | 237.81 | 2.72 | 708.49 | 2.22 | 1 |
| 1050 | 785337 | AA476502 | 0.01 | 2.19 | 219.32 | 2.43 | 652.56 | 2.96 | 1 |
| 1051 | 840937 | AA486538 | 0.01 | 1.97 | 197.29 | 2.91 | 586.88 | 2.07 | 1 |
| 1052 | 1325605 | AA875888 | 0.04 | 6.87 | 172.23 | 0.55 | 515.39 | 0.74 | 1 |
| 1053 | 784116 | AA432058 | 0.04 | 6.56 | 154.17 | 1.62 | 458.06 | 2.83 | 1 |
| 1054 | 796350 | AA456130 | 0.49 | 73.56 | 149.45 | 0.82 | 446.45 | 1.09 | 1 |
| 1055 | 743027 | AA406070 | 0.34 | 49.37 | 146.97 | 1.13 | 438.73 | 1.04 | 1 |
| 1056 | 742564 | AA401342 | 0.78 | 69.16 | 88.80 | 1.24 | 264.42 | 0.74 | 1 |
| 1057 | 796334 | AA461320 | 0.09 | 7.42 | 80.84 | 0.85 | 241.37 | 0.30 | 1 |
| 1058 | 769542 | AA426212 | 0.05 | 3.61 | 75.00 | 1.50 | 221.03 | 2.47 | 1 |
| 1059 | 47963 | H11642 | 0.12 | 8.93 | 74.54 | 1.79 | 220.07 | 1.77 | 1 |
| 1060 | 897722 | AA598983 | 0.23 | 16.59 | 73.32 | 0.77 | 218.92 | 0.29 | 1 |
| 1061 | 270603 | N33323 | 0.33 | 23.96 | 73.13 | 0.60 | 218.05 | 0.76 | 1 |
| 1062 | 726439 | AA399245 | 0.02 | 1.43 | 72.56 | 2.48 | 214.82 | 0.38 | 1 |
| 1063 | 44007 | H05961 | 0.04 | 2.83 | 72.19 | 2.70 | 212.83 | 1.04 | 1 |
| 1064 | 37986 | R81395 | 0.01 | 0.65 | 64.60 | 1.54 | 191.25 | 1.00 | 1 |
| 1065 | 586645 | AA133554 | 0.06 | 3.72 | 59.54 | 1.07 | 176.58 | 0.97 | 1 |
| 1066 | 427693 | AA002166 | 0.35 | 20.73 | 59.22 | 1.25 | 175.32 | 1.10 | 1 |
| 1067 | 594683 | AA171784 | 0.31 | 18.15 | 58.59 | 1.85 | 173.57 | 0.36 | 1 |
| 1068 | 49131 | H16537 | 0.03 | 1.84 | 57.46 | 2.01 | 168.03 | 2.35 | 1 |
| 1069 | 782353 | AA432278 | 0.26 | 14.22 | 55.03 | 1.13 | 162.70 | 1.26 | 1 |
| 1070 | 810959 | AA459400 | 0.07 | 3.65 | 54.74 | 0.86 | 161.00 | 2.37 | 1 |
| 1071 | 838877 | AA481806 | 0.09 | 4.65 | 51.74 | 0.97 | 153.57 | 0.68 | 1 |
| 1072 | 382278 | AA001219 | 0.02 | 0.94 | 51.03 | 2.74 | 149.67 | 0.69 | 1 |
| 1073 | 1048794 | AA621324 | 0.15 | 7.71 | 50.64 | 0.39 | 151.46 | 0.07 | 1 |
| 1074 | 193087 | H47076 | 0.02 | 1.05 | 50.20 | 1.48 | 148.64 | 0.48 | 1 |
| 1075 | 360355 | AA013353 | 0.19 | 9.24 | 48.61 | 0.82 | 143.96 | 1.06 | 1 |
| 1076 | 726934 | AA398364 | 0.70 | 32.83 | 46.77 | 2.69 | 135.65 | 1.96 | 1 |
| 1077 | 796723 | AA443140 | 0.03 | 1.25 | 46.43 | 3.00 | 135.09 | 1.21 | 1 |
| 1078 | 838600 | AA456951 | 0.27 | 12.55 | 46.12 | 0.95 | 137.23 | 0.19 | 1 |
| 1079 | 344834 | W70264 | 0.02 | 0.77 | 44.22 | 1.23 | 130.86 | 0.57 | 1 |
| 1080 | 271141 | N34482 | 0.03 | 1.29 | 43.90 | 0.79 | 128.29 | 2.63 | 1 |
| 1081 | 773209 | AA425743 | 0.48 | 21.09 | 43.69 | 1.18 | 129.35 | 0.54 | 1 |
| 1082 | 625516 | AA187595 | 0.02 | 0.63 | 41.89 | 0.47 | 122.87 | 2.32 | 1 |
| 1083 | 46055 | H08850 | 0.11 | 4.73 | 41.59 | 122.01 | 1.33 | 1.42 | 1 |
| 1084 | 416608 | W86779 | 1.03 | 42.43 | 41.07 | 0.72 | 122.05 | 0.44 | 1 |
| 1085 | 323251 | W42996 | 0.10 | 4.04 | 40.88 | 0.41 | 120.12 | 2.13 | 1 |
| 1086 | 284701 | N64840 | 0.39 | 15.59 | 40.15 | 118.16 | 1.22 | 1.07 | 1 |
| 1087 | 255859 | N27841 | 0.04 | 1.72 | 40.03 | 1.06 | 117.01 | 2.03 | 1 |
| 1088 | 268979 | N26083 | 0.58 | 23.11 | 39.71 | 1.18 | 117.08 | 0.87 | 1 |
| 1089 | 51799 | H23524 | 0.36 | 14.33 | 39.51 | 118.37 | 0.03 | 0.13 | 1 |
| 1090 | 417780 | W88720 | 0.28 | 10.92 | 39.20 | 1.76 | 114.00 | 1.83 | 1 |
| 1091 | 609436 | AA180453 | 0.78 | 30.27 | 38.61 | 1.15 | 113.82 | 0.84 | 1 |
| 1092 | 838853 | AA481788 | 2.21 | 82.43 | 37.26 | 0.85 | 110.55 | 0.39 | 1 |
| 1093 | 276905 | N34943 | 0.06 | 2.07 | 36.82 | 0.97 | 109.30 | 0.18 | 1 |
| 1094 | 897262 | AA677643 | 0.36 | 13.35 | 36.69 | 0.73 | 1.02 | 108.31 | 1 |
| 1095 | 810459 | AA457138 | 0.45 | 16.34 | 36.32 | 0.68 | 106.62 | 1.65 | 1 |
| 1096 | 128461 | R10140 | 0.05 | 1.94 | 36.21 | 0.12 | 106.86 | 1.65 | 1 |
| 1097 | 254545 | N23865 | 0.30 | 10.68 | 35.93 | 1.55 | 104.16 | 2.06 | 1 |
| 1098 | 279176 | N46845 | 0.39 | 13.40 | 34.13 | 1.05 | 99.56 | 1.78 | 1 |
| 1099 | 284408 | N52158 | 0.01 | 0.34 | 33.51 | 1.40 | 98.13 | 1.00 | 1 |
| 1100 | 204686 | H57136 | 0.01 | 0.32 | 32.37 | 1.00 | 1.00 | 95.10 | 1 |
| 1101 | 75866 | AA449300 | 0.01 | 0.32 | 31.55 | 1.86 | 90.43 | 2.37 | 1 |
| 1102 | 282161 | N48271 | 1.19 | 37.35 | 31.48 | 1.16 | 92.44 | 0.86 | 1 |
| 1103 | 592403 | AA159497 | 0.01 | 0.31 | 31.48 | 2.74 | 90.70 | 1.00 | 1 |
| 1104 | 526567 | AA128407 | 0.04 | 1.21 | 31.06 | 1.08 | 91.71 | 0.39 | 1 |
| 1105 | 452848 | AA704816 | 0.18 | 5.31 | 28.99 | 1.73 | 84.23 | 1.00 | 1 |
| 1106 | 329059 | W43028 | 0.06 | 1.64 | 28.51 | 2.92 | 82.35 | 0.25 | 1 |
| 1107 | 564231 | AA121387 | 0.02 | 0.63 | 28.14 | 2.11 | 79.62 | 2.69 | 1 |
| 1108 | 1323328 | AA872602 | 0.01 | 0.36 | 27.29 | 2.21 | 78.91 | 0.76 | 1 |
| 1109 | 285581 | N66454 | 0.92 | 25.14 | 27.26 | 1.10 | 79.11 | 1.56 | 1 |
| 1110 | 27152 | R37289 | 0.20 | 5.54 | 27.13 | 1.24 | 79.32 | 0.82 | 1 |
| 1111 | 773678 | AA433910 | 0.07 | 1.91 | 27.12 | 1.91 | 76.73 | 2.73 | 1 |
| 1112 | 1323448 | AA873604 | 0.48 | 12.79 | 26.76 | 1.43 | 1.76 | 77.08 | 1 |
| 1113 | 813654 | AA447751 | 0.09 | 2.37 | 26.59 | 1.74 | 76.96 | 1.06 | 1 |
| 1114 | 970901 | AA776041 | 1.02 | 26.72 | 26.31 | 1.00 | 75.29 | 2.63 | 1 |
| 1115 | 299603 | N74889 | 1.29 | 33.43 | 25.93 | 2.60 | 73.65 | 1.55 | 1 |
| 1116 | 27544 | R40057 | 0.01 | 0.26 | 25.76 | 1.21 | 73.28 | 2.80 | 1 |
| 1117 | 767739 | AA418028 | 0.15 | 3.89 | 25.55 | 0.75 | 74.73 | 1.17 | 1 |
| 1118 | 773293 | AA425214 | 0.09 | 2.31 | 25.16 | 0.29 | 74.38 | 0.80 | 1 |
| 1119 | 731115 | AA417280 | 0.37 | 9.24 | 25.13 | 1.28 | 72.44 | 1.68 | 1 |
| 1120 | 48363 | H15050 | 0.18 | 4.63 | 25.02 | 1.92 | 70.67 | 2.46 | 1 |
| 1121 | 38260 | R49462 | 0.22 | 5.49 | 24.82 | 1.38 | 71.10 | 2.00 | 1 |
| 1122 | 251961 | H97508 | 2.40 | 59.39 | 24.72 | 0.41 | 71.45 | 2.30 | 1 |
| 1123 | 285682 | N67571 | 0.65 | 16.06 | 24.67 | 1.19 | 71.41 | 1.42 | 1 |
| 1124 | 284569 | N64774 | 0.01 | 0.25 | 24.66 | 2.32 | 70.65 | 1.00 | 1 |
| 1125 | 784154 | AA432096 | 0.03 | 0.84 | 24.57 | 0.29 | 71.20 | 2.23 | 1 |
| 1126 | 1466893 | AA884321 | 0.44 | 10.63 | 24.27 | 2.54 | 68.08 | 2.18 | 1 |
| 1127 | 127711 | R09504 | 1.57 | 37.42 | 23.82 | 1.01 | 69.82 | 0.64 | 1 |
| 1128 | 429660 | AA011570 | 0.44 | 10.35 | 23.68 | 1.52 | 68.34 | 1.17 | 1 |
| 1129 | 364454 | AA022668 | 0.53 | 12.38 | 23.36 | 1.28 | 67.64 | 1.17 | 1 |
| 1130 | 757235 | AA426030 | 1.21 | 27.72 | 22.90 | 1.35 | 64.57 | 2.77 | 1 |
| 1131 | 731154 | AA417211 | 0.28 | 6.49 | 22.78 | 1.39 | 65.48 | 1.45 | 1 |
| 1132 | 823796 | AA490264 | 0.42 | 9.38 | 22.60 | 0.83 | 66.20 | 0.77 | 1 |
| 1133 | 196475 | R92512 | 0.10 | 2.19 | 22.38 | 1.64 | 65.41 | 0.10 | 1 |
| 1134 | 742890 | AA406220 | 0.64 | 14.30 | 22.28 | 1.16 | 64.81 | 0.87 | 1 |
| 1135 | 285364 | N66346 | 0.77 | 17.18 | 22.23 | 0.97 | 64.73 | 0.97 | 1 |
| 1136 | 328745 | W45403 | 0.04 | 0.81 | 22.20 | 0.70 | 63.34 | 2.57 | 1 |
| 1137 | 273024 | N36389 | 0.38 | 8.49 | 22.17 | 0.84 | 64.28 | 1.38 | 1 |
| 1138 | 344747 | W74696 | 0.19 | 4.05 | 21.69 | 1.59 | 62.07 | 1.40 | 1 |
| 1139 | 730377 | AA470073 | 0.59 | 12.75 | 21.68 | 1.18 | 63.23 | 0.63 | 1 |
| 1140 | 253534 | H89563 | 1.42 | 29.99 | 21.17 | 0.56 | 62.26 | 0.68 | 1 |
| 1141 | 289865 | N62079 | 0.51 | 10.68 | 20.87 | 1.21 | 59.72 | 1.70 | 1 |
| 1142 | 502593 | AA136049 | 0.01 | 0.18 | 20.81 | 1.29 | 2.06 | 59.09 | 1 |
| 1143 | 38244 | R49436 | 0.08 | 1.66 | 20.63 | 1.10 | 60.67 | 0.12 | 1 |
| 1144 | 33045 | R19478 | 0.15 | 3.00 | 20.35 | 1.33 | 58.64 | 1.09 | 1 |
| 1145 | 37901 | R59304 | 0.27 | 5.45 | 20.32 | 0.81 | 59.31 | 0.84 | 1 |
| 1146 | 1466551 | AA885141 | 0.25 | 5.04 | 19.89 | 0.96 | 56.55 | 2.16 | 1 |
| 1147 | 259865 | N29851 | 0.64 | 12.57 | 19.79 | 1.44 | 57.49 | 0.46 | 1 |
| 1148 | 306841 | N91921 | 0.60 | 11.90 | 19.67 | 1.24 | 57.17 | 0.59 | 1 |
| 1149 | 525478 | AA065042 | 0.01 | 0.19 | 19.20 | 1.00 | 55.61 | 1.00 | 1 |
| 1150 | 609209 | AA167120 | 0.44 | 8.40 | 19.11 | 1.58 | 53.79 | 1.97 | 1 |
| 1151 | 344618 | W74725 | 0.01 | 0.19 | 19.00 | 1.10 | 1.00 | 54.90 | 1 |
| 1152 | 731016 | AA421256 | 0.01 | 0.19 | 18.85 | 2.52 | 53.32 | 0.71 | 1 |
| 1153 | 109275 | T80832 | 0.65 | 12.10 | 18.68 | 0.98 | 54.30 | 0.76 | 1 |
| 1154 | 32551 | R43189 | 0.03 | 0.48 | 18.29 | 0.36 | 54.14 | 0.38 | 1 |
| 1155 | 1461725 | AA884397 | 0.83 | 14.96 | 18.05 | 0.31 | 53.43 | 0.41 | 1 |
| 1158 | 1342650 | AA725397 | 0.13 | 2.32 | 18.02 | 1.27 | 51.75 | 1.04 | 1 |
| 1157 | 262060 | H99075 | 0.98 | 17.51 | 17.94 | 0.55 | 51.86 | 1.41 | 1 |
| 1158 | 743828 | AA634379 | 0.40 | 7.11 | 17.66 | 50.03 | 1.71 | 1.23 | 1 |
| 1159 | 1048694 | AA620607 | 0.39 | 6.75 | 17.52 | 1.35 | 49.38 | 1.83 | 1 |
| 1160 | 626186 | AA188710 | 0.08 | 1.37 | 17.44 | 2.13 | 49.70 | 0.50 | 1 |
| 1161 | 745330 | AA625641 | 0.18 | 3.05 | 17.28 | 0.40 | 50.90 | 0.53 | 1 |
| 1162 | 767449 | AA418000 | 0.47 | 8.13 | 17.25 | 49.43 | 0.87 | 1.45 | 1 |
| 1163 | 451871 | AA706935 | 0.11 | 1.84 | 17.14 | 0.80 | 48.99 | 1.64 | 1 |
| 1164 | 42225 | R60731 | 0.36 | 6.13 | 17.05 | 1.89 | 46.96 | 2.29 | 1 |
| 1165 | 31869 | R43017 | 0.10 | 1.60 | 16.83 | 0.36 | 49.51 | 0.63 | 1 |
| 1166 | 1468260 | AA884926 | 0.35 | 5.82 | 16.65 | 45.76 | 1.50 | 2.70 | 1 |
| 1167 | 281003 | N50880 | 0.22 | 3.57 | 16.52 | 48.82 | 0.05 | 0.69 | 1 |
| 1168 | 731422 | AA412403 | 0.37 | 6.03 | 16.52 | 1.51 | 46.45 | 1.59 | 1 |
| 1169 | 279102 | N51709 | 0.32 | 5.21 | 16.51 | 1.72 | 45.77 | 2.06 | 1 |
| 1170 | 261989 | H98678 | 0.23 | 3.70 | 16.42 | 0.92 | 46.79 | 1.55 | 1 |
| 1171 | 1030808 | AA620301 | 0.68 | 11.22 | 16.40 | 1.01 | 47.42 | 0.77 | 1 |
| 1172 | 137853 | R68409 | 0.31 | 4.98 | 16.18 | 1.38 | 46.41 | 0.74 | 1 |
| 1173 | 276438 | N40202 | 0.91 | 14.63 | 16.13 | 1.23 | 46.01 | 1.16 | 1 |
| 1174 | 627401 | AA190825 | 0.10 | 1.63 | 16.07 | 1.38 | 46.29 | 0.53 | 1 |
| 1175 | 38677 | R51506 | 0.27 | 4.23 | 15.67 | 0.93 | 44.76 | 1.30 | 1 |
| 1176 | 768445 | AA495938 | 0.35 | 5.43 | 15.59 | 1.31 | 42.94 | 2.52 | 1 |
| 1177 | 299162 | N75473 | 0.01 | 0.16 | 15.56 | 1.32 | 1.00 | 44.37 | 1 |
| 1178 | 1626197 | AI005321 | 0.18 | 2.75 | 15.49 | 1.79 | 42.61 | 2.06 | 1 |
| 1179 | 285370 | N66348 | 0.55 | 8.40 | 15.37 | 1.18 | 43.72 | 1.23 | 1 |
| 1180 | 323599 | W44338 | 0.50 | 7.61 | 15.30 | 2.79 | 42.03 | 1.07 | 1 |
| 1181 | 1412504 | AA845168 | 0.25 | 3.82 | 15.28 | 1.07 | 44.40 | 0.38 | 1 |
| 1182 | 731091 | AA421170 | 0.56 | 8.56 | 15.26 | 1.08 | 43.90 | 0.79 | 1 |
| 1183 | 549157 | AA083514 | 0.85 | 12.92 | 15.12 | 1.48 | 42.90 | 0.97 | 1 |
| 1184 | 1388373 | AA844124 | 0.33 | 5.05 | 15.11 | 0.85 | 43.54 | 0.94 | 1 |
| 1185 | 731444 | AA412435 | 0.50 | 7.34 | 14.83 | 1.04 | 42.32 | 1.13 | 1 |
| 1186 | 30986 | R41782 | 0.27 | 4.03 | 14.76 | 1.17 | 41.82 | 1.29 | 1 |
| 1187 | 279616 | N48302 | 0.58 | 8.55 | 14.65 | 1.26 | 42.02 | 0.68 | 1 |
| 1188 | 280270 | N47954 | 0.53 | 7.71 | 14.55 | 1.33 | 40.40 | 1.92 | 1 |
| 1189 | 1474149 | AA911971 | 0.02 | 0.34 | 14.54 | 2.10 | 41.08 | 0.42 | 1 |
| 1190 | 80242 | T64323 | 1.49 | 21.55 | 14.50 | 1.51 | 41.04 | 0.95 | 1 |
| 1191 | 562080 | AA211483 | 0.05 | 0.78 | 14.36 | 0.98 | 40.96 | 1.16 | 1 |
| 1192 | 450375 | AA703660 | 0.13 | 1.87 | 14.30 | 1.45 | 41.37 | 0.08 | 1 |
| 1193 | 784242 | AA446882 | 0.59 | 8.37 | 14.22 | 0.76 | 41.22 | 0.67 | 1 |
| 1194 | 25933 | R36969 | 0.01 | 0.14 | 13.99 | 2.13 | 1.37 | 38.45 | 1 |
| 1195 | 42433 | R61189 | 0.15 | 2.05 | 13.98 | 1.19 | 39.26 | 1.48 | 1 |
| 1196 | 45501 | H08397 | 0.09 | 1.31 | 13.90 | 40.60 | 0.11 | 0.99 | 1 |
| 1197 | 811819 | AA463483 | 0.58 | 8.10 | 13.87 | 0.91 | 39.18 | 1.53 | 1 |
| 1198 | 284845 | N66724 | 0.77 | 10.61 | 13.85 | 1.22 | 39.02 | 1.31 | 1 |
| 1199 | 744417 | AA621218 | 0.01 | 0.14 | 13.84 | 2.64 | 2.34 | 36.52 | 1 |
| 1200 | 328689 | W45330 | 0.70 | 9.64 | 13.79 | 1.10 | 39.01 | 1.27 | 1 |
| 1201 | 342089 | W60894 | 1.08 | 14.87 | 13.73 | 0.91 | 39.43 | 0.85 | 1 |
| 1202 | 38015 | R61556 | 0.17 | 2.31 | 13.67 | 0.69 | 39.92 | 0.39 | 1 |
| 1203 | 810511 | AA464541 | 1.09 | 14.75 | 13.59 | 1.51 | 36.40 | 2.87 | 1 |
| 1204 | 262968 | H99722 | 0.62 | 8.43 | 13.52 | 0.89 | 38.96 | 0.71 | 1 |
| 1205 | 549035 | AA083207 | 0.21 | 2.77 | 13.50 | 0.86 | 39.39 | 0.25 | 1 |
| 1206 | 612782 | AA181723 | 0.57 | 7.69 | 13.50 | 0.89 | 38.18 | 1.42 | 1 |
| 1207 | 730888 | AA417031 | 0.95 | 12.65 | 13.37 | 0.86 | 38.68 | 0.57 | 1 |
| 1208 | 34901 | R45114 | 0.27 | 3.60 | 13.37 | 1.42 | 38.10 | 0.57 | 1 |
| 1209 | 243410 | N38992 | 0.90 | 12.03 | 13.35 | 1.22 | 38.03 | 0.79 | 1 |
| 1210 | 730398 | AA469939 | 1.01 | 13.46 | 13.30 | 0.95 | 38.31 | 0.65 | 1 |
| 1211 | 592778 | AA158211 | 0.02 | 0.26 | 13.29 | 2.26 | 35.14 | 2.46 | 1 |
| 1212 | 730827 | AA417017 | 0.50 | 6.68 | 13.27 | 1.42 | 37.10 | 1.29 | 1 |
| 1213 | 283045 | N51290 | 0.59 | 7.81 | 13.26 | 0.73 | 38.34 | 0.72 | 1 |
| 1214 | 796130 | AA460967 | 0.34 | 4.53 | 13.22 | 1.19 | 37.93 | 0.53 | 1 |
| 1215 | 591814 | AA143467 | 0.09 | 1.19 | 13.15 | 0.42 | 38.93 | 0.11 | 1 |
| 1216 | 308031 | N92310 | 0.22 | 2.89 | 13.01 | 1.76 | 35.65 | 1.60 | 1 |
| 1217 | 742666 | AA400273 | 1.08 | 14.04 | 12.99 | 1.47 | 36.87 | 0.65 | 1 |
| 1218 | 1032072 | AA609887 | 0.55 | 7.09 | 12.96 | 1.33 | 36.05 | 1.51 | 1 |
| 1219 | 415820 | W84815 | 0.63 | 8.15 | 12.91 | 0.97 | 36.14 | 1.62 | 1 |
| 1220 | 358046 | W94591 | 0.52 | 6.66 | 12.84 | 1.34 | 36.21 | 0.98 | 1 |
| 1221 | 80186 | T64192 | 1.07 | 13.65 | 12.78 | 1.20 | 36.50 | 0.65 | 1 |
| 1222 | 454317 | AA677165 | 0.33 | 4.20 | 12.72 | 35.38 | 1.48 | 1.30 | 1 |
| 1223 | 768111 | AA418988 | 0.67 | 8.45 | 12.60 | 1.20 | 35.01 | 1.58 | 1 |
| 1224 | 299024 | N75394 | 0.46 | 5.81 | 12.58 | 1.04 | 35.31 | 1.39 | 1 |
| 1225 | 510136 | AA053239 | 0.01 | 0.13 | 12.52 | 1.00 | 35.57 | 1.00 | 1 |
| 1226 | 41300 | R56642 | 0.21 | 2.68 | 12.49 | 0.99 | 35.21 | 1.26 | 1 |
| 1227 | 278629 | N66203 | 0.49 | 6.08 | 12.49 | 1.77 | 33.25 | 2.43 | 1 |
| 1228 | 1466883 | AA884317 | 0.67 | 8.34 | 12.47 | 1.55 | 35.13 | 0.71 | 1 |
| 1229 | 208413 | H62162 | 0.32 | 3.96 | 12.44 | 35.07 | 1.49 | 0.76 | 1 |
| 1230 | 782279 | AA431750 | 0.52 | 6.39 | 12.37 | 1.54 | 34.77 | 0.79 | 1 |
| 1231 | 296448 | N74623 | 0.02 | 0.19 | 12.35 | 0.66 | 0.66 | 35.74 | 1 |
| 1232 | 511233 | AA088701 | 0.18 | 2.21 | 12.29 | 1.20 | 34.01 | 1.66 | 1 |
| 1233 | 359040 | W92278 | 0.83 | 10.12 | 12.20 | 0.77 | 34.90 | 0.95 | 1 |
| 1234 | 366795 | AA029428 | 0.48 | 5.89 | 12.16 | 1.40 | 33.59 | 1.50 | 1 |
| 1235 | 588159 | AA135222 | 1.78 | 21.58 | 12.10 | 2.01 | 32.82 | 1.46 | 1 |
| 1236 | 742671 | AA400281 | 0.65 | 7.77 | 12.04 | 1.11 | 33.72 | 1.28 | 1 |
| 1237 | 1031375 | AA609138 | 0.88 | 7.93 | 11.98 | 0.97 | 34.33 | 0.64 | 1 |
| 1238 | 129862 | R17096 | 0.01 | 0.12 | 11.96 | 1.00 | 2.56 | 32.33 | 1 |
| 1239 | 813697 | AA453769 | 0.80 | 9.57 | 11.96 | 0.87 | 33.75 | 1.25 | 1 |
| 1240 | 625684 | AA186335 | 0.87 | 10.22 | 11.73 | 0.82 | 33.81 | 0.58 | 1 |
| 1241 | 289594 | N62780 | 1.35 | 15.85 | 11.70 | 1.91 | 32.40 | 0.80 | 1 |
| 1242 | 203488 | H55955 | 0.08 | 0.90 | 11.63 | 1.22 | 32.62 | 1.06 | 1 |
| 1243 | 785788 | AA449745 | 0.29 | 3.36 | 11.63 | 0.78 | 32.51 | 1.61 | 1 |
| 1244 | 346615 | W74471 | 0.30 | 3.53 | 11.62 | 1.68 | 31.51 | 1.68 | 1 |
| 1245 | 1458160 | AA862465 | 0.23 | 2.61 | 11.47 | 33.19 | 0.28 | 0.95 | 1 |
| 1246 | 433350 | AA700604 | 6.42 | 73.54 | 11.46 | 31.50 | 1.22 | 1.66 | 1 |
| 1247 | 112559 | T91244 | 0.30 | 3.38 | 11.38 | 1.20 | 32.53 | 0.40 | 1 |
| 1248 | 753368 | AA411682 | 0.18 | 2.02 | 11.35 | 0.58 | 33.21 | 0.27 | 1 |
| 1249 | 264449 | N21233 | 0.04 | 0.41 | 11.26 | 1.70 | 30.58 | 1.51 | 1 |
| 1250 | 786073 | AA448672 | 0.66 | 7.40 | 11.26 | 1.20 | 31.68 | 0.90 | 1 |
| 1251 | 221828 | H92234 | 0.06 | 0.68 | 11.23 | 2.84 | 29.73 | 1.13 | 1 |
| 1252 | 384015 | AA702640 | 0.36 | 4.08 | 11.23 | 32.64 | 0.83 | 0.32 | 1 |
| 1253 | 730344 | AA469922 | 0.48 | 5.33 | 11.16 | 1.35 | 30.72 | 1.42 | 1 |
| 1254 | 258063 | N30328 | 0.87 | 9.68 | 11.15 | 1.11 | 31.28 | 1.05 | 1 |
| 1255 | 627292 | AA191495 | 0.07 | 0.79 | 11.09 | 1.32 | 30.94 | 0.99 | 1 |
| 1256 | 626462 | AA188999 | 1.08 | 11.98 | 11.08 | 1.26 | 31.46 | 0.52 | 1 |
| 1257 | 823618 | AA496949 | 0.66 | 7.28 | 11.05 | 0.91 | 31.48 | 0.76 | 1 |
| 1258 | 270274 | N33550 | 0.32 | 3.51 | 11.04 | 1.23 | 31.25 | 0.63 | 1 |
| 1259 | 28774 | R40244 | 0.14 | 1.56 | 11.00 | 1.13 | 31.52 | 0.36 | 1 |
| 1260 | 50582 | H17038 | 0.30 | 3.26 | 10.99 | 0.83 | 31.48 | 0.67 | 1 |
| 1261 | 31478 | R42763 | 0.24 | 2.62 | 10.95 | 0.80 | 29.45 | 2.58 | 1 |
| 1262 | 83358 | T68445 | 1.46 | 15.93 | 10.89 | 30.68 | 1.01 | 0.98 | 1 |
| 1263 | 289725 | N59278 | 0.83 | 9.08 | 10.89 | 0.97 | 30.86 | 0.84 | 1 |
| 1264 | 262313 | H99460 | 0.46 | 5.00 | 10.76 | 0.81 | 29.68 | 1.80 | 1 |
| 1265 | 29251 | R41389 | 0.12 | 1.34 | 10.75 | 1.66 | 29.36 | 1.25 | 1 |
| 1266 | 280413 | N47214 | 0.51 | 5.45 | 10.74 | 0.92 | 29.74 | 1.56 | 1 |
| 1267 | 811785 | AA483463 | 0.24 | 2.56 | 10.70 | 1.06 | 29.70 | 1.34 | 1 |
| 1268 | 44387 | H06525 | 0.25 | 2.63 | 10.68 | 2.06 | 0.06 | 29.91 | 1 |
| 1269 | 38517 | R51236 | 0.11 | 1.13 | 10.62 | 0.73 | 31.04 | 0.09 | 1 |
| 1270 | 742616 | AA401482 | 0.40 | 4.27 | 10.60 | 1.65 | 29.17 | 0.97 | 1 |
| 1271 | 629968 | AA219315 | 0.24 | 2.53 | 10.52 | 0.75 | 30.56 | 0.24 | 1 |
| 1272 | 35308 | R45402 | 0.45 | 4.74 | 10.45 | 1.01 | 29.08 | 1.26 | 1 |
| 1273 | 687579 | AA236561 | 0.67 | 6.94 | 10.41 | 1.26 | 29.40 | 0.56 | 1 |
| 1274 | 784177 | AA446658 | 1.77 | 18.36 | 10.40 | 1.13 | 28.01 | 2.05 | 1 |
| 1275 | 730294 | AA412512 | 0.33 | 3.36 | 10.32 | 1.20 | 28.15 | 1.60 | 1 |
| 1276 | 1466549 | AA885140 | 0.44 | 4.50 | 10.26 | 1.12 | 28.46 | 1.20 | 1 |
| 1277 | 72663 | T50397 | 0.01 | 0.10 | 10.26 | 1.20 | 28.58 | 1.00 | 1 |
| 1278 | 795723 | AA460289 | 0.34 | 3.44 | 10.24 | 1.33 | 28.32 | 1.07 | 1 |
| 1279 | 773423 | AA426038 | 0.15 | 1.53 | 10.20 | 0.52 | 29.62 | 0.45 | 1 |
| 1280 | 795170 | AA453466 | 0.42 | 4.33 | 10.20 | 1.31 | 28.06 | 1.21 | 1 |
| 1281 | 282000 | N51107 | 1.38 | 14.09 | 10.19 | 1.38 | 28.36 | 0.82 | 1 |
| 1282 | 743519 | AA609415 | 0.53 | 5.32 | 10.01 | 1.20 | 27.58 | 1.26 | 1 |
| 1283 | 898038 | AA598943 | 0.35 | 3.47 | 10.00 | 1.65 | 26.99 | 1.36 | 1 |
| 1284 | 364950 | AA024459 | 0.60 | 6.03 | 9.97 | 1.33 | 27.67 | 0.90 | 1 |
| 1285 | 44303 | H06380 | 0.73 | 7.21 | 9.92 | 1.63 | 26.24 | 1.89 | 1 |
| 1286 | 251685 | H96738 | 0.46 | 4.57 | 9.87 | 0.49 | 28.25 | 0.88 | 1 |
| 1287 | 344010 | W70242 | 0.67 | 6.60 | 9.84 | 1.03 | 26.16 | 2.33 | 1 |
| 1288 | 795540 | AA459652 | 0.45 | 4.44 | 9.84 | 1.30 | 27.33 | 0.90 | 1 |
| 1289 | 121633 | T97599 | 1.25 | 12.27 | 9.82 | 1.16 | 27.36 | 0.95 | 1 |
| 1290 | 347020 | W79499 | 0.55 | 5.41 | 9.80 | 1.15 | 27.43 | 0.81 | 1 |
| 1291 | 1325751 | AA873089 | 0.56 | 5.49 | 9.76 | 1.25 | 26.98 | 1.04 | 1 |
| 1292 | 742874 | AA406213 | 0.76 | 7.41 | 9.75 | 1.14 | 27.37 | 0.73 | 1 |
| 1293 | 283619 | N52876 | 0.50 | 4.86 | 9.73 | 1.02 | 27.09 | 1.08 | 1 |
| 1294 | 47793 | H11730 | 0.18 | 1.71 | 9.69 | 1.83 | 0.40 | 26.83 | 1 |
| 1295 | 201931 | H52446 | 0.16 | 1.53 | 9.66 | 1.01 | 26.66 | 1.31 | 1 |
| 1296 | 43422 | H05626 | 0.16 | 1.56 | 9.65 | 0.74 | 27.97 | 0.24 | 1 |
| 1297 | 253314 | H89331 | 0.71 | 6.78 | 9.61 | 1.31 | 26.48 | 1.05 | 1 |
| 1298 | 781506 | AA432141 | 0.52 | 4.97 | 9.56 | 1.25 | 26.99 | 0.43 | 1 |
| 1299 | 647397 | AA199717 | 1.00 | 9.52 | 9.55 | 2.24 | 24.20 | 2.20 | 1 |
| 1300 | 43072 | R61871 | 0.50 | 4.77 | 9.52 | 0.87 | 26.65 | 1.03 | 1 |
| 1301 | 491565 | AA115076 | 3.49 | 33.20 | 9.51 | 2.58 | 2.81 | 23.15 | 1 |
| 1302 | 796398 | AA459941 | 0.36 | 3.37 | 9.40 | 24.67 | 2.59 | 0.93 | 1 |
| 1303 | 299347 | N70654 | 0.71 | 6.72 | 9.40 | 1.17 | 26.29 | 0.74 | 1 |
| 1304 | 51908 | H23555 | 0.08 | 0.72 | 9.38 | 2.22 | 23.30 | 2.61 | 1 |
| 1305 | 26844 | R39901 | 0.18 | 1.69 | 9.34 | 1.48 | 23.87 | 2.67 | 1 |
| 1306 | 43815 | H05741 | 0.10 | 0.94 | 9.34 | 0.68 | 27.22 | 0.12 | 1 |
| 1307 | 84141 | T71042 | 0.01 | 0.09 | 9.32 | 1.90 | 1.00 | 25.05 | 1 |
| 1308 | 502664 | AA127069 | 0.82 | 7.66 | 9.28 | 0.96 | 24.01 | 2.88 | 1 |
| 1309 | 1292207 | AA705858 | 0.15 | 1.40 | 9.28 | 1.03 | 25.00 | 1.80 | 1 |
| 1310 | 343871 | W69649 | 0.02 | 0.19 | 9.26 | 1.55 | 25.75 | 0.49 | 1 |
| 1311 | 67397 | T49325 | 0.01 | 0.09 | 9.22 | 1.00 | 25.66 | 1.00 | 1 |
| 1312 | 346642 | W74633 | 0.33 | 3.06 | 9.20 | 1.38 | 25.56 | 0.65 | 1 |
| 1313 | 399319 | AA774645 | 0.26 | 2.38 | 9.14 | 1.09 | 24.09 | 2.23 | 1 |
| 1314 | 272288 | N35603 | 0.40 | 3.63 | 9.07 | 1.18 | 24.82 | 1.22 | 1 |
| 1315 | 29424 | R41357 | 0.37 | 3.37 | 9.00 | 1.07 | 24.08 | 1.86 | 1 |
| 1316 | 757265 | AA426113 | 2.19 | 19.69 | 8.99 | 22.82 | 2.38 | 1.78 | 1 |
| 1317 | 811050 | AA485429 | 0.10 | 0.91 | 8.99 | 1.61 | 23.59 | 1.78 | 1 |
| 1318 | 257608 | N27272 | 0.71 | 6.33 | 8.97 | 1.04 | 24.68 | 1.18 | 1 |
| 1319 | 839903 | AA490058 | 0.05 | 0.48 | 8.96 | 1.57 | 24.09 | 1.23 | 1 |
| 1320 | 343977 | W70074 | 0.43 | 3.83 | 8.96 | 0.96 | 24.14 | 1.79 | 1 |
| 1321 | 757352 | AA437107 | 1.00 | 8.93 | 8.93 | 0.81 | 25.33 | 0.66 | 1 |
| 1322 | 502527 | AA156873 | 0.10 | 0.87 | 8.91 | 0.35 | 23.64 | 2.72 | 1 |
| 1323 | 471859 | AA035144 | 0.01 | 0.07 | 8.87 | 1.26 | 24.08 | 1.26 | 1 |
| 1324 | 796448 | AA459983 | 0.04 | 0.37 | 8.82 | 1.28 | 23.71 | 1.47 | 1 |
| 1325 | 45578 | H07920 | 0.26 | 2.31 | 8.75 | 2.26 | 21.71 | 2.29 | 1 |
| 1326 | 530814 | AA070226 | 1.22 | 10.67 | 8.72 | 22.34 | 2.00 | 1.83 | 1 |
| 1327 | 278809 | N66580 | 1.31 | 11.41 | 8.70 | 0.22 | 25.76 | 0.13 | 1 |
| 1328 | 782575 | AA447522 | 0.58 | 5.00 | 8.69 | 0.91 | 0.02 | 25.13 | 1 |
| 1329 | 969568 | AA772803 | 0.09 | 0.78 | 8.69 | 2.33 | 21.36 | 2.36 | 1 |
| 1330 | 796408 | AA459945 | 0.09 | 0.78 | 8.67 | 0.13 | 24.92 | 0.95 | 1 |
| 1331 | 898162 | AA598538 | 3.55 | 30.73 | 8.65 | 1.27 | 0.53 | 24.14 | 1 |
| 1332 | 786560 | AA452125 | 0.42 | 3.60 | 8.64 | 0.83 | 23.05 | 2.05 | 1 |
| 1333 | 448379 | AA778206 | 0.45 | 3.84 | 8.54 | 0.90 | 24.06 | 0.67 | 1 |
| 1334 | 773276 | AA425319 | 0.32 | 2.73 | 8.52 | 1.00 | 23.01 | 1.55 | 1 |
| 1335 | 53371 | R15779 | 0.11 | 0.89 | 8.50 | 1.79 | 21.50 | 2.22 | 1 |
| 1336 | 284479 | N52350 | 0.04 | 0.30 | 6.50 | 0.49 | 0.28 | 24.73 | 1 |
| 1337 | 1372140 | AA974495 | 4.41 | 37.45 | 8.50 | 2.69 | 20.01 | 2.79 | 1 |
| 1338 | 37283 | R51064 | 0.23 | 1.91 | 8.48 | 1.21 | 22.84 | 1.38 | 1 |
| 1339 | 774751 | AA442095 | 1.04 | 8.81 | 8.46 | 0.57 | 1.42 | 23.39 | 1 |
| 1340 | 324122 | W46577 | 0.42 | 3.53 | 8.45 | 1.56 | 22.84 | 0.94 | 1 |
| 1341 | 811845 | AA463643 | 0.44 | 3.73 | 8.44 | 0.84 | 23.77 | 0.70 | 1 |
| 1342 | 285261 | N63153 | 2.41 | 20.33 | 8.43 | 0.63 | 22.41 | 2.24 | 1 |
| 1343 | 1376853 | AA812996 | 0.35 | 2.92 | 8.41 | 1.45 | 20.90 | 2.88 | 1 |
| 1344 | 613070 | AA181646 | 2.03 | 17.03 | 8.40 | 0.60 | 23.97 | 0.62 | 1 |
| 1345 | 726699 | AA398262 | 0.24 | 2.03 | 8.39 | 1.21 | 23.00 | 0.97 | 1 |
| 1346 | 31056 | R42490 | 0.23 | 1.94 | 8.38 | 1.38 | 23.12 | 0.65 | 1 |
| 1347 | 509495 | AA047338 | 2.23 | 18.69 | 8.38 | 1.99 | 2.77 | 20.37 | 1 |
| 1348 | 1466418 | AA885471 | 0.47 | 3.93 | 8.34 | 0.24 | 24.31 | 0.47 | 1 |
| 1349 | 448014 | AA702785 | 0.17 | 1.42 | 8.31 | 0.78 | 23.22 | 0.94 | 1 |
| 1350 | 291129 | N72150 | 0.33 | 2.74 | 8.29 | 0.34 | 24.02 | 0.50 | 1 |
| 1351 | 340898 | W57712 | 1.23 | 10.19 | 8.28 | 2.68 | 19.19 | 2.96 | 1 |
| 1352 | 284220 | N53534 | 0.29 | 2.38 | 8.24 | 1.18 | 22.37 | 1.19 | 1 |
| 1353 | 782847 | AA448283 | 0.53 | 4.33 | 8.21 | 1.14 | 22.28 | 1.22 | 1 |
| 1354 | 450574 | AA704255 | 1.05 | 8.62 | 8.21 | 0.80 | 23.03 | 0.80 | 1 |
| 1355 | 35503 | R45672 | 0.22 | 1.81 | 8.20 | 1.45 | 21.45 | 1.71 | 1 |
| 1356 | 32381 | R43452 | 0.28 | 2.30 | 8.20 | 22.17 | 1.81 | 0.62 | 1 |
| 1357 | 731337 | AA416782 | 0.42 | 3.42 | 8.16 | 1.13 | 21.62 | 1.74 | 1 |
| 1358 | 1461654 | AA885311 | 1.34 | 10.88 | 8.15 | 2.95 | 0.15 | 21.36 | 1 |
| 1359 | 786504 | AA452095 | 2.20 | 17.90 | 8.15 | 2.90 | 0.49 | 21.07 | 1 |
| 1360 | 743992 | AA629020 | 0.36 | 2.94 | 8.11 | 1.08 | 1.49 | 21.75 | 1 |
| 1361 | 742702 | AA401380 | 0.24 | 1.93 | 8.07 | 1.00 | 22.43 | 0.77 | 1 |
| 1362 | 341861 | W60795 | 0.45 | 3.62 | 8.03 | 1.35 | 21.23 | 1.51 | 1 |
| 1363 | 595318 | AA164301 | 0.57 | 4.55 | 8.03 | 1.14 | 20.14 | 2.80 | 1 |
| 1364 | 788629 | AA449823 | 0.05 | 0.40 | 8.02 | 1.92 | 19.87 | 2.29 | 1 |
| 1365 | 781404 | AA430202 | 0.35 | 2.83 | 8.01 | 0.90 | 22.05 | 1.07 | 1 |
| 1366 | 49836 | H29207 | 0.57 | 4.52 | 7.98 | 1.66 | 21.87 | 0.42 | 1 |
| 1367 | 42009 | R60713 | 0.03 | 0.25 | 7.97 | 2.14 | 20.92 | 0.85 | 1 |
| 1368 | 34641 | R44404 | 0.19 | 1.55 | 7.96 | 0.99 | 22.36 | 0.53 | 1 |
| 1369 | 286661 | N67891 | 0.65 | 5.16 | 7.96 | 1.05 | 21.01 | 1.82 | 1 |
| 1370 | 230591 | H75459 | 0.52 | 4.12 | 7.96 | 20.12 | 1.68 | 2.07 | 1 |
| 1371 | 283158 | N51343 | 1.40 | 11.10 | 7.95 | 1.35 | 21.75 | 0.74 | 1 |
| 1372 | 843058 | AA488604 | 0.15 | 1.17 | 7.92 | 0.77 | 22.04 | 0.96 | 1 |
| 1373 | 1031919 | AA609749 | 1.09 | 8.58 | 7.85 | 1.33 | 20.70 | 1.52 | 1 |
| 1374 | 280213 | N49186 | 0.74 | 5.83 | 7.83 | 1.09 | 21.42 | 0.97 | 1 |
| 1375 | 757257 | AA426092 | 0.23 | 1.78 | 7.77 | 0.65 | 21.62 | 1.05 | 1 |
| 1376 | 280266 | N47952 | 0.53 | 4.12 | 7.74 | 0.73 | 21.04 | 1.44 | 1 |
| 1377 | 1030781 | AA609005 | 0.90 | 6.90 | 7.71 | 1.21 | 20.67 | 1.23 | 1 |
| 1378 | 786669 | AA451890 | 0.67 | 5.09 | 7.63 | 1.43 | 20.64 | 0.82 | 1 |
| 1379 | 69272 | T54342 | 0.01 | 0.08 | 7.62 | 0.56 | 21.29 | 1.00 | 1 |
| 1380 | 772408 | AA405532 | 1.49 | 11.32 | 7.60 | 1.33 | 20.21 | 1.27 | 1 |
| 1381 | 784272 | AA447476 | 0.14 | 1.07 | 7.56 | 1.39 | 19.58 | 1.72 | 1 |
| 1382 | 278516 | N62817 | 0.56 | 4.20 | 7.56 | 0.99 | 20.96 | 0.71 | 1 |
| 1383 | 418158 | W85931 | 0.80 | 6.05 | 7.55 | 0.56 | 21.82 | 0.48 | 1 |
| 1384 | 511117 | AA088231 | 0.06 | 0.46 | 7.54 | 2.35 | 18.03 | 2.23 | 1 |
| 1385 | 283874 | N52589 | 0.10 | 0.79 | 7.53 | 1.68 | 18.23 | 2.69 | 1 |
| 1386 | 746321 | AA481397 | 0.44 | 3.30 | 7.49 | 0.67 | 1.28 | 20.54 | 1 |
| 1387 | 129392 | R11236 | 0.05 | 0.41 | 7.48 | 1.30 | 1.87 | 19.28 | 1 |
| 1388 | 40296 | R52082 | 0.84 | 6.24 | 7.41 | 1.45 | 1.27 | 19.52 | 1 |
| 1389 | 245768 | N55266 | 0.01 | 0.10 | 7.41 | 0.76 | 0.44 | 21.03 | 1 |
| 1390 | 595695 | AA173189 | 3.54 | 26.15 | 7.38 | 1.82 | 0.14 | 20.18 | 1 |
| 1391 | 852829 | AA668178 | 0.01 | 0.07 | 7.35 | 2.78 | 1.00 | 18.26 | 1 |
| 1392 | 795199 | AA453585 | 0.54 | 3.92 | 7.33 | 0.86 | 20.91 | 0.41 | 1 |
| 1393 | 626551 | AA187982 | 0.48 | 3.50 | 7.31 | 1.32 | 18.62 | 2.00 | 1 |
| 1394 | 823772 | AA490240 | 0.19 | 1.40 | 7.31 | 1.72 | 18.94 | 1.25 | 1 |
| 1395 | 1049293 | AA620760 | 0.43 | 3.14 | 7.28 | 1.18 | 19.21 | 1.44 | 1 |
| 1396 | 277322 | N57475 | 0.31 | 2.27 | 7.27 | 1.76 | 18.29 | 1.76 | 1 |
| 1397 | 42387 | R59990 | 0.20 | 1.44 | 7.26 | 1.12 | 19.38 | 1.29 | 1 |
| 1398 | 307174 | N91797 | 1.14 | 8.29 | 7.25 | 1.22 | 19.82 | 0.69 | 1 |
| 1399 | 417296 | W88786 | 1.03 | 7.41 | 7.17 | 0.92 | 20.04 | 0.54 | 1 |
| 1400 | 399152 | AA733105 | 0.09 | 0.63 | 7.15 | 0.64 | 19.41 | 1.40 | 1 |
| 1401 | 503583 | AA131240 | 0.58 | 4.11 | 7.14 | 1.07 | 19.54 | 0.81 | 1 |
| 1402 | 284851 | N64814 | 0.28 | 1.86 | 7.12 | 2.96 | 16.61 | 1.79 | 1 |
| 1403 | 345081 | W74802 | 0.19 | 1.32 | 7.12 | 20.99 | 0.29 | 0.08 | 1 |
| 1404 | 415814 | W84778 | 0.21 | 1.50 | 7.11 | 16.41 | 2.76 | 2.15 | 1 |
| 1405 | 754008 | AA478965 | 0.25 | 1.74 | 7.08 | 0.51 | 19.69 | 1.05 | 1 |
| 1406 | 39161 | R51865 | 0.22 | 1.59 | 7.07 | 1.30 | 18.84 | 1.08 | 1 |
| 1407 | 277974 | N63436 | 0.10 | 0.70 | 7.04 | 1.41 | 18.55 | 1.15 | 1 |
| 1408 | 729954 | AA412046 | 0.91 | 6.35 | 7.00 | 0.96 | 19.09 | 0.95 | 1 |
| 1409 | 813735 | AA453795 | 0.36 | 2.53 | 6.97 | 0.62 | 18.94 | 1.36 | 1 |
| 1410 | 796442 | AA459980 | 2.01 | 13.98 | 6.97 | 18.11 | 0.81 | 1.98 | 1 |
| 1411 | 626842 | AA191424 | 0.44 | 3.06 | 8.94 | 16.68 | 2.00 | 2.14 | 1 |
| 1412 | 281936 | N51069 | 0.98 | 6.77 | 6.93 | 1.05 | 18.56 | 1.19 | 1 |
| 1413 | 291790 | N67861 | 6.24 | 43.23 | 6.93 | 1.55 | 0.20 | 19.02 | 1 |
| 1414 | 838818 | AA457675 | 0.35 | 2.38 | 6.90 | 1.44 | 17.91 | 1.36 | 1 |
| 1415 | 781489 | AA432144 | 0.45 | 3.11 | 6.90 | 1.16 | 17.72 | 1.82 | 1 |
| 1416 | 283703 | N50733 | 0.56 | 3.88 | 6.90 | 1.39 | 18.47 | 0.84 | 1 |
| 1417 | 39266 | R51872 | 0.10 | 0.66 | 6.90 | 1.01 | 18.92 | 0.76 | 1 |
| 1418 | 590120 | AA156022 | 0.68 | 4.70 | 6.89 | 1.06 | 18.61 | 0.99 | 1 |
| 1419 | 27508 | R40037 | 0.24 | 1.65 | 6.88 | 1.26 | 17.58 | 1.81 | 1 |
| 1420 | 730742 | AA435988 | 0.75 | 5.14 | 6.87 | 0.90 | 19.11 | 0.59 | 1 |
| 1421 | 213634 | H72107 | 0.85 | 5.80 | 6.83 | 1.64 | 17.15 | 1.70 | 1 |
| 1422 | 50618 | H17516 | 0.28 | 1.93 | 6.83 | 1.21 | 18.38 | 0.89 | 1 |
| 1423 | 1030854 | AA621750 | 0.72 | 4.90 | 6.81 | 1.04 | 18.44 | 0.93 | 1 |
| 1424 | 584847 | AA129217 | 1.04 | 7.07 | 6.81 | 1.18 | 18.33 | 0.91 | 1 |
| 1425 | 302073 | N89783 | 0.29 | 2.00 | 6.80 | 1.28 | 17.77 | 1.36 | 1 |
| 1426 | 730146 | AA412477 | 0.43 | 2.92 | 6.80 | 0.39 | 19.47 | 0.55 | 1 |
| 1427 | 307660 | N92901 | 0.65 | 4.43 | 6.80 | 0.67 | 17.89 | 1.84 | 1 |
| 1428 | 795334 | AA454170 | 0.80 | 5.43 | 6.80 | 0.76 | 19.11 | 0.52 | 1 |
| 1429 | 489572 | AA101640 | 0.24 | 1.64 | 6.78 | 1.09 | 15.56 | 2.69 | 1 |
| 1430 | 68052 | T52820 | 0.01 | 0.07 | 6.77 | 1.00 | 1.00 | 18.30 | 1 |
| 1431 | 593652 | AA166703 | 0.10 | 0.71 | 6.77 | 0.97 | 18.08 | 1.24 | 1 |
| 1432 | 505227 | AA142923 | 0.38 | 2.59 | 6.75 | 1.20 | 17.97 | 1.09 | 1 |
| 1433 | 257167 | N30562 | 0.70 | 4.72 | 6.75 | 0.76 | 18.29 | 1.19 | 1 |
| 1434 | 795829 | AA461507 | 0.84 | 5.68 | 6.74 | 1.07 | 18.42 | 0.74 | 1 |
| 1435 | 1412398 | AA844998 | 0.38 | 2.58 | 6.73 | 0.92 | 18.56 | 0.73 | 1 |
| 1436 | 811046 | AA485427 | 0.02 | 0.11 | 6.72 | 0.63 | 18.92 | 0.63 | 1 |
| 1437 | 1422424 | AA827369 | 0.51 | 3.42 | 6.70 | 0.51 | 18.93 | 0.57 | 1 |
| 1438 | 781046 | AA446451 | 7.77 | 51.77 | 6.66 | 1.79 | 0.83 | 17.37 | 1 |
| 1439 | 376040 | AA040332 | 0.79 | 5.28 | 6.66 | 1.14 | 16.50 | 2.35 | 1 |
| 1440 | 260216 | N32095 | 1.81 | 12.09 | 6.66 | 0.51 | 18.64 | 0.83 | 1 |
| 1441 | 788234 | AA454080 | 0.81 | 5.41 | 6.65 | 0.86 | 18.31 | 0.76 | 1 |
| 1442 | 247862 | N58276 | 0.52 | 3.47 | 6.63 | 1.27 | 18.25 | 0.38 | 1 |
| 1443 | 27333 | R37079 | 0.18 | 1.22 | 6.62 | 1.84 | 16.69 | 1.34 | 1 |
| 1444 | 280308 | N47075 | 0.27 | 1.82 | 6.60 | 1.26 | 15.70 | 2.84 | 1 |
| 1445 | 1048586 | AA608856 | 2.91 | 19.16 | 6.59 | 1.72 | 0.22 | 17.84 | 1 |
| 1446 | 460398 | AA677522 | 0.01 | 0.07 | 6.58 | 2.45 | 1.00 | 16.28 | 1 |
| 1447 | 727275 | AA401695 | 0.35 | 2.30 | 6.56 | 0.85 | 17.83 | 1.02 | 1 |
| 1448 | 788240 | AA454082 | 0.09 | 0.60 | 6.56 | 1.01 | 18.57 | 0.11 | 1 |
| 1449 | 341061 | W58291 | 0.55 | 3.63 | 6.55 | 1.48 | 17.20 | 0.97 | 1 |
| 1450 | 1055144 | AA621363 | 0.69 | 4.51 | 6.55 | 1.90 | 16.06 | 1.68 | 1 |
| 1451 | 796369 | AA458148 | 1.37 | 8.94 | 6.54 | 1.12 | 18.17 | 0.34 | 1 |
| 1452 | 35620 | R45632 | 0.21 | 1.36 | 6.53 | 1.39 | 17.90 | 0.30 | 1 |
| 1453 | 346897 | W78168 | 0.48 | 3.15 | 6.53 | 0.81 | 18.01 | 0.76 | 1 |
| 1454 | 1461161 | AA868038 | 0.48 | 3.12 | 6.52 | 16.55 | 1.51 | 1.50 | 1 |
| 1455 | 361656 | W96273 | 0.28 | 1.81 | 6.52 | 2.01 | 2.64 | 14.90 | 1 |
| 1456 | 325182 | W49619 | 0.64 | 4.15 | 6.51 | 1.28 | 17.38 | 0.87 | 1 |
| 1457 | 815051 | AA465168 | 0.86 | 5.61 | 6.51 | 0.84 | 2.07 | 16.61 | 1 |
| 1458 | 1417935 | AA878739 | 0.96 | 6.21 | 6.50 | 0.51 | 18.14 | 0.84 | 1 |
| 1459 | 384939 | AA708686 | 0.28 | 1.84 | 6.48 | 1.01 | 17.01 | 1.43 | 1 |
| 1460 | 745493 | AA625990 | 0.44 | 2.83 | 6.48 | 16.43 | 1.84 | 1.17 | 1 |
| 1461 | 38528 | R51261 | 0.16 | 1.04 | 6.48 | 1.90 | 14.61 | 2.93 | 1 |
| 1462 | 809611 | AA458487 | 0.01 | 0.04 | 6.48 | 0.87 | 17.11 | 1.44 | 1 |
| 1463 | 290162 | N63286 | 1.54 | 9.86 | 6.41 | 1.53 | 14.87 | 2.84 | 1 |
| 1464 | 505454 | AA156433 | 1.28 | 8.15 | 6.39 | 1.42 | 16.90 | 0.86 | 1 |
| 1465 | 782838 | AA448270 | 1.12 | 7.19 | 6.39 | 0.49 | 0.43 | 18.24 | 1 |
| 1466 | 731040 | AA421271 | 0.30 | 1.88 | 6.37 | 1.80 | 15.24 | 2.08 | 1 |
| 1467 | 342039 | W60288 | 0.57 | 3.64 | 6.35 | 1.27 | 16.26 | 1.53 | 1 |
| 1468 | 795254 | AA453993 | 0.85 | 5.43 | 6.35 | 0.85 | 17.18 | 1.03 | 1 |
| 1469 | 487383 | AA046705 | 1.96 | 12.45 | 6.34 | 1.25 | 16.71 | 1.08 | 1 |
| 1470 | 823578 | AA497040 | 0.25 | 1.60 | 6.31 | 1.15 | 16.50 | 1.28 | 1 |
| 1471 | 743025 | AA406069 | 1.15 | 7.26 | 6.29 | 0.65 | 16.91 | 1.31 | 1 |
| 1472 | 37404 | R51100 | 0.23 | 1.45 | 6.28 | 1.76 | 15.30 | 1.79 | 1 |
| 1473 | 731198 | AA417355 | 0.21 | 1.33 | 6.27 | 1.18 | 16.03 | 1.60 | 1 |
| 1474 | 303023 | N91566 | 0.61 | 3.82 | 6.24 | 1.16 | 16.63 | 0.92 | 1 |
| 1475 | 786154 | AA448855 | 0.48 | 2.98 | 6.22 | 0.99 | 16.75 | 0.94 | 1 |
| 1476 | 361587 | AA016980 | 0.17 | 1.04 | 6.21 | 1.81 | 2.34 | 14.49 | 1 |
| 1477 | 727137 | AA398757 | 1.15 | 7.16 | 6.20 | 0.84 | 17.00 | 0.78 | 1 |
| 1478 | 665033 | AA194646 | 0.11 | 0.70 | 6.19 | 1.87 | 14.44 | 2.25 | 1 |
| 1479 | 461420 | AA704943 | 0.41 | 2.50 | 6.17 | 15.37 | 1.89 | 1.26 | 1 |
| 1480 | 591540 | AA158735 | 0.02 | 0.15 | 6.17 | 0.41 | 0.41 | 17.69 | 1 |
| 1481 | 731050 | AA421276 | 0.99 | 6.12 | 6.15 | 1.25 | 16.18 | 1.04 | 1 |
| 1482 | 162734 | H27545 | 0.18 | 1.11 | 6.15 | 15.27 | 1.11 | 2.06 | 1 |
| 1483 | 289903 | N64597 | 0.81 | 4.98 | 6.14 | 1.14 | 15.91 | 1.39 | 1 |
| 1484 | 135900 | R33609 | 1.42 | 8.74 | 6.14 | 1.63 | 2.30 | 14.49 | 1 |
| 1485 | 609530 | AA180163 | 0.47 | 2.86 | 6.13 | 1.01 | 15.63 | 1.75 | 1 |
| 1486 | 782668 | AA447574 | 0.34 | 2.08 | 6.11 | 1.81 | 15.22 | 1.30 | 1 |
| 1487 | 277631 | N45983 | 0.50 | 3.06 | 6.11 | 1.07 | 16.26 | 1.00 | 1 |
| 1488 | 1569463 | AA973927 | 1.82 | 11.09 | 6.09 | 13.75 | 1.73 | 2.80 | 1 |
| 1489 | 255286 | N23882 | 0.14 | 0.87 | 6.08 | 0.71 | 17.19 | 0.35 | 1 |
| 1490 | 461965 | AA779972 | 0.40 | 2.40 | 6.07 | 0.69 | 16.27 | 1.25 | 1 |
| 1491 | 742928 | AA405181 | 0.48 | 2.91 | 6.07 | 1.59 | 15.36 | 1.25 | 1 |
| 1492 | 897641 | AA496792 | 0.25 | 1.54 | 6.07 | 1.05 | 16.43 | 0.72 | 1 |
| 1493 | 1469857 | AA865150 | 0.65 | 3.94 | 6.05 | 1.37 | 15.22 | 1.56 | 1 |
| 1494 | 770989 | AA427404 | 0.03 | 0.16 | 6.05 | 0.85 | 2.37 | 14.92 | 1 |
| 1495 | 214624 | H71242 | 0.52 | 3.17 | 6.05 | 2.52 | 14.36 | 1.26 | 1 |
| 1496 | 280214 | N49181 | 1.02 | 6.12 | 6.01 | 1.92 | 13.20 | 2.90 | 1 |
| 1497 | 46715 | H10011 | 0.27 | 1.59 | 6.00 | 2.75 | 1.81 | 13.42 | 1 |
| 1498 | 324345 | W47585 | 0.01 | 0.06 | 5.98 | 1.76 | 15.18 | 1.00 | 1 |
| 1499 | 838500 | AA457528 | 0.34 | 2.05 | 5.98 | 1.18 | 15.89 | 0.86 | 1 |
| 1500 | 743184 | AA401409 | 0.79 | 4.68 | 5.95 | 1.32 | 15.78 | 0.75 | 1 |
| 1501 | 266148 | N21576 | 0.67 | 3.98 | 5.95 | 0.60 | 0.01 | 17.23 | 1 |
| 1502 | 772938 | AA479928 | 0.36 | 2.17 | 5.95 | 1.28 | 16.22 | 0.34 | 1 |
| 1503 | 795339 | AA453256 | 0.44 | 2.60 | 5.93 | 1.27 | 15.41 | 1.12 | 1 |
| 1504 | 878097 | AA775405 | 0.25 | 1.49 | 5.93 | 0.77 | 14.53 | 2.48 | 1 |
| 1505 | 788107 | AA448825 | 0.79 | 4.69 | 5.91 | 1.10 | 15.90 | 0.74 | 1 |
| 1506 | 85840 | H2235 | 2.06 | 12.14 | 5.91 | 0.45 | 0.54 | 16.74 | 1 |
| 1507 | 1055727 | AA628132 | 0.23 | 1.35 | 5.90 | 0.72 | 15.08 | 1.90 | 1 |
| 1508 | 51749 | H24329 | 0.37 | 2.18 | 5.89 | 17.10 | 0.16 | 0.40 | 1 |
| 1509 | 767461 | AA418004 | 0.36 | 2.11 | 5.88 | 1.67 | 13.14 | 2.85 | 1 |
| 1510 | 46183 | H09616 | 0.04 | 0.21 | 5.88 | 2.42 | 14.95 | 0.28 | 1 |
| 1511 | 856388 | AA774578 | 6.44 | 37.89 | 5.88 | 1.55 | 3.00 | 13.10 | 1 |
| 1512 | 298162 | N70791 | 0.35 | 2.08 | 5.87 | 1.16 | 15.23 | 1.22 | 1 |
| 1513 | 586923 | AA130955 | 1.18 | 6.90 | 5.87 | 0.79 | 15.93 | 0.88 | 1 |
| 1514 | 310054 | N95476 | 1.04 | 6.10 | 5.87 | 1.30 | 15.63 | 0.67 | 1 |
| 1515 | 813807 | AA447724 | 0.25 | 1.46 | 5.86 | 1.23 | 15.07 | 1.27 | 1 |
| 1516 | 184256 | H43707 | 0.42 | 2.46 | 5.81 | 14.09 | 2.49 | 0.87 | 1 |
| 1517 | 1055636 | AA620927 | 0.37 | 2.13 | 5.81 | 0.88 | 15.63 | 0.91 | 1 |
| 1518 | 279583 | N48901 | 0.47 | 2.71 | 5.76 | 1.07 | 13.52 | 2.69 | 1 |
| 1519 | 277785 | N49619 | 0.28 | 1.60 | 5.76 | 1.36 | 14.88 | 1.05 | 1 |
| 1520 | 28106 | R40780 | 0.85 | 4.87 | 5.75 | 1.77 | 0.66 | 14.81 | 1 |
| 1521 | 417461 | W88999 | 1.03 | 5.93 | 5.74 | 1.04 | 15.61 | 0.56 | 1 |
| 1522 | 254564 | N23652 | 1.09 | 6.22 | 5.72 | 1.37 | 14.85 | 0.96 | 1 |
| 1523 | 757190 | AA443958 | 4.29 | 24.55 | 5.72 | 0.26 | 16.15 | 0.77 | 1 |
| 1524 | 586798 | AA133469 | 0.06 | 0.32 | 5.72 | 1.68 | 15.29 | 0.18 | 1 |
| 1525 | 270681 | N33366 | 0.13 | 0.76 | 5.70 | 1.44 | 15.60 | 0.07 | 1 |
| 1526 | 283702 | N50729 | 0.55 | 3.16 | 5.70 | 1.10 | 14.20 | 1.79 | 1 |
| 1527 | 840404 | AA485653 | 4.80 | 27.34 | 5.69 | 1.08 | 2.35 | 13.64 | 1 |
| 1528 | 594806 | AA171426 | 1.09 | 6.19 | 5.67 | 0.88 | 15.05 | 1.08 | 1 |
| 1529 | 251517 | H96605 | 0.52 | 2.93 | 5.66 | 1.05 | 14.61 | 1.31 | 1 |
| 1530 | 797048 | AA463225 | 0.41 | 2.33 | 5.65 | 1.01 | 13.08 | 2.88 | 1 |
| 1531 | 415191 | W95118 | 0.57 | 3.19 | 5.63 | 2.86 | 11.46 | 2.58 | 1 |
| 1532 | 272951 | N33778 | 0.01 | 0.06 | 5.61 | 1.00 | 1.00 | 14.84 | 1 |
| 1533 | 341095 | W58209 | 0.52 | 2.90 | 5.59 | 1.11 | 14.64 | 1.04 | 1 |
| 1534 | 898288 | AA598975 | 0.18 | 1.02 | 5.59 | 1.31 | 14.07 | 1.39 | 1 |
| 1535 | 951102 | AA620463 | 1.12 | 6.24 | 5.59 | 1.17 | 12.89 | 2.72 | 1 |
| 1536 | 281243 | N51002 | 0.19 | 1.04 | 5.59 | 14.87 | 0.45 | 1.44 | 1 |
| 1537 | 44287 | H06249 | 0.07 | 0.37 | 5.58 | 2.63 | 13.78 | 0.34 | 1 |
| 1538 | 758370 | AA404356 | 0.40 | 2.21 | 5.58 | 1.89 | 14.42 | 0.43 | 1 |
| 1539 | 625816 | AA186613 | 0.44 | 2.46 | 5.58 | 1.47 | 12.33 | 2.93 | 1 |
| 1540 | 358212 | W95414 | 0.53 | 2.93 | 5.57 | 1.33 | 14.57 | 0.80 | 1 |
| 1541 | 898218 | AA598601 | 1.30 | 7.23 | 5.56 | 2.92 | 2.90 | 10.87 | 1 |
| 1542 | 148469 | H12338 | 0.08 | 0.42 | 5.56 | 13.10 | 1.81 | 1.76 | 1 |
| 1543 | 133864 | R28660 | 0.12 | 0.65 | 5.55 | 1.63 | 14.66 | 0.34 | 1 |
| 1544 | 280633 | N50432 | 1.05 | 5.82 | 5.54 | 0.90 | 15.17 | 0.54 | 1 |
| 1545 | 179753 | H51549 | 0.01 | 0.06 | 5.53 | 1.11 | 1.00 | 14.48 | 1 |
| 1546 | 742589 | AA401472 | 0.56 | 3.10 | 5.52 | 1.11 | 14.52 | 0.93 | 1 |
| 1547 | 742590 | AA401470 | 0.03 | 0.16 | 5.52 | 1.82 | 12.03 | 2.71 | 1 |
| 1548 | 434845 | AA703147 | 1.16 | 6.39 | 5.50 | 0.93 | 1.14 | 14.43 | 1 |
| 1549 | 344505 | W73523 | 0.10 | 0.57 | 5.48 | 0.59 | 15.22 | 0.84 | 1 |
| 1550 | 1031552 | AA609284 | 0.57 | 3.15 | 5.48 | 1.22 | 14.23 | 0.98 | 1 |
| 1551 | 121409 | T96913 | 0.74 | 4.04 | 5.48 | 0.88 | 14.78 | 0.77 | 1 |
| 1552 | 45922 | H09541 | 0.20 | 1.09 | 5.47 | 1.35 | 13.47 | 1.59 | 1 |
| 1553 | 358200 | W95409 | 0.79 | 4.31 | 5.45 | 1.46 | 14.20 | 0.69 | 1 |
| 1554 | 415264 | W92011 | 1.18 | 6.45 | 5.45 | 1.27 | 1.33 | 13.75 | 1 |
| 1555 | 203544 | H56028 | 2.30 | 12.51 | 5.44 | 2.64 | 2.97 | 10.73 | 1 |
| 1556 | 782611 | AA447547 | 0.07 | 0.40 | 5.44 | 2.49 | 13.71 | 0.14 | 1 |
| 1557 | 951101 | AA620472 | 0.04 | 0.22 | 5.44 | 0.78 | 15.03 | 0.52 | 1 |
| 1558 | 306121 | N90514 | 0.72 | 3.92 | 5.43 | 1.86 | 13.09 | 1.34 | 1 |
| 1559 | 429773 | AA009763 | 0.05 | 0.25 | 5.42 | 1.92 | 14.13 | 0.22 | 1 |
| 1560 | 795585 | AA459702 | 0.06 | 0.33 | 5.42 | 2.61 | 11.67 | 1.99 | 1 |
| 1561 | 27098 | R36989 | 0.05 | 0.28 | 5.42 | 0.59 | 15.56 | 0.11 | 1 |
| 1562 | 795788 | AA460328 | 0.01 | 0.03 | 5.41 | 12.59 | 1.81 | 1.81 | 1 |
| 1563 | 281756 | N48078 | 0.86 | 4.66 | 5.39 | 1.04 | 14.19 | 0.95 | 1 |
| 1564 | 280672 | N47425 | 0.58 | 3.15 | 5.39 | 1.04 | 14.02 | 1.12 | 1 |
| 1565 | 609052 | AA176607 | 0.61 | 3.29 | 5.39 | 1.07 | 13.69 | 1.40 | 1 |
| 1566 | 645332 | AA205803 | 0.40 | 2.17 | 5.39 | 2.60 | 12.04 | 1.52 | 1 |
| 1567 | 755904 | AA496544 | 2.48 | 13.33 | 5.39 | 1.48 | 2.43 | 12.24 | 1 |
| 1568 | 240273 | H89713 | 0.27 | 1.45 | 5.37 | 1.30 | 14.21 | 0.60 | 1 |
| 1569 | 825583 | AA504617 | 0.01 | 0.05 | 5.36 | 2.88 | 10.35 | 2.86 | 1 |
| 1570 | 127242 | R08292 | 0.39 | 2.10 | 5.36 | 1.17 | 13.41 | 1.50 | 1 |
| 1571 | 462468 | AA699895 | 0.25 | 1.33 | 5.34 | 13.49 | 1.23 | 1.31 | 1 |
| 1572 | 562158 | AA211565 | 0.75 | 4.00 | 5.34 | 0.99 | 13.90 | 1.13 | 1 |
| 1573 | 270766 | N29796 | 0.15 | 0.82 | 5.34 | 1.08 | 13.96 | 0.97 | 1 |
| 1574 | 742887 | AA406226 | 0.42 | 2.21 | 5.32 | 1.04 | 14.31 | 0.61 | 1 |
| 1575 | 128058 | R09729 | 1.29 | 6.86 | 5.32 | 0.65 | 0.18 | 15.12 | 1 |
| 1576 | 346359 | W74216 | 0.90 | 4.78 | 5.31 | 0.68 | 14.89 | 0.38 | 1 |
| 1577 | 489535 | AA098867 | 1.87 | 9.92 | 5.31 | 0.82 | 13.87 | 1.43 | 1 |
| 1578 | 297878 | N70038 | 3.78 | 20.08 | 5.31 | 0.75 | 14.20 | 0.97 | 1 |
| 1579 | 840444 | AA485714 | 0.82 | 4.34 | 5.31 | 0.50 | 14.65 | 0.76 | 1 |
| 1580 | 251591 | 1196643 | 0.03 | 0.18 | 5.31 | 0.30 | 1.68 | 13.96 | 1 |
| 1581 | 745072 | AA626275 | 0.35 | 1.87 | 5.30 | 12.38 | 1.48 | 2.04 | 1 |
| 1582 | 378242 | AA777024 | 2.14 | 11.30 | 5.29 | 1.37 | 13.86 | 0.63 | 1 |
| 1583 | 42120 | R59818 | 0.39 | 2.08 | 5.29 | 0.85 | 14.51 | 0.49 | 1 |
| 1584 | 278386 | N66085 | 1.09 | 5.76 | 5.28 | 2.04 | 11.84 | 1.96 | 1 |
| 1585 | 627306 | AA191518 | 0.56 | 2.96 | 5.28 | 1.40 | 13.61 | 0.83 | 1 |
| 1586 | 124148 | R01739 | 0.70 | 3.67 | 5.28 | 14.20 | 1.16 | 0.47 | 1 |
| 1587 | 757222 | AA498149 | 0.66 | 3.47 | 5.28 | 14.23 | 0.22 | 1.38 | 1 |
| 1588 | 595623 | AA167338 | 1.00 | 5.25 | 5.27 | 1.12 | 13.86 | 0.83 | 1 |
| 1589 | 743452 | AA609368 | 0.84 | 4.41 | 5.25 | 1.10 | 13.60 | 1.05 | 1 |
| 1590 | 454698 | AA677200 | 0.56 | 2.92 | 5.25 | 1.26 | 13.15 | 1.33 | 1 |
| 1591 | 280412 | N47208 | 0.02 | 0.13 | 5.24 | 0.49 | 0.41 | 14.83 | 1 |
| 1592 | 360844 | AA015819 | 1.79 | 9.37 | 5.24 | 0.59 | 13.78 | 1.34 | 1 |
| 1593 | 758309 | AA404269 | 1.08 | 5.67 | 5.23 | 0.33 | 12.98 | 2.39 | 1 |
| 1594 | 462963 | AA682439 | 0.43 | 2.23 | 5.23 | 1.88 | 12.77 | 1.04 | 1 |
| 1595 | 665154 | AA195668 | 0.44 | 2.30 | 5.23 | 1.87 | 12.97 | 0.84 | 1 |
| 1596 | 592497 | AA161466 | 0.22 | 1.16 | 5.22 | 0.13 | 15.02 | 0.51 | 1 |
| 1597 | 878417 | AA670359 | 0.18 | 0.94 | 5.21 | 0.41 | 12.55 | 2.66 | 1 |
| 1598 | 462729 | AA705184 | 0.48 | 2.50 | 5.20 | 1.26 | 11.43 | 2.91 | 1 |
| 1599 | 284157 | N20602 | 0.11 | 0.56 | 5.20 | 0.63 | 14.87 | 0.09 | 1 |
| 1600 | 283890 | N50787 | 0.52 | 2.70 | 5.19 | 1.54 | 12.74 | 1.28 | 1 |
| 1601 | 51011 | H19242 | 0.70 | 3.62 | 5.18 | 14.21 | 0.93 | 0.40 | 1 |
| 1602 | 162533 | H27986 | 2.59 | 13.39 | 5.18 | 1.16 | 12.97 | 1.40 | 1 |
| 1603 | 376435 | AA039713 | 1.11 | 5.75 | 5.17 | 0.73 | 12.99 | 1.80 | 1 |
| 1604 | 1475972 | AA872979 | 0.27 | 1.38 | 5.17 | 1.17 | 12.54 | 1.80 | 1 |
| 1605 | 742898 | AA405809 | 2.49 | 12.87 | 5.17 | 0.92 | 13.61 | 0.96 | 1 |
| 1606 | 1031736 | AA609594 | 0.75 | 3.90 | 5.16 | 1.37 | 12.96 | 1.16 | 1 |
| 1607 | 53081 | R16259 | 0.69 | 3.54 | 5.16 | 1.80 | 13.19 | 0.50 | 1 |
| 1608 | 1470278 | AA866029 | 0.51 | 2.63 | 5.16 | 0.43 | 14.23 | 0.82 | 1 |
| 1609 | 767414 | AA417915 | 0.59 | 3.04 | 5.15 | 1.12 | 13.32 | 1.02 | 1 |
| 1610 | 341065 | W58191 | 0.52 | 2.68 | 5.15 | 1.67 | 12.49 | 1.28 | 1 |
| 1611 | 233712 | H79035 | 0.42 | 2.14 | 5.14 | 10.92 | 2.33 | 2.19 | 1 |
| 1612 | 781447 | AA428659 | 0.63 | 3.22 | 5.14 | 1.32 | 13.03 | 1.07 | 1 |
| 1613 | 1030764 | AA608974 | 0.71 | 3.64 | 5.13 | 0.80 | 13.91 | 0.68 | 1 |
| 1614 | 196189 | R92281 | 1.98 | 10.18 | 5.13 | 10.73 | 2.45 | 2.21 | 1 |
| 1615 | 279827 | N40976 | 1.11 | 5.71 | 5.13 | 1.08 | 13.20 | 1.10 | 1 |
| 1616 | 1292121 | AA707615 | 0.27 | 1.36 | 5.13 | 10.73 | 1.75 | 2.91 | 1 |
| 1617 | 490649 | AA101777 | 2.39 | 12.23 | 5.12 | 11.35 | 2.00 | 2.00 | 1 |
| 1618 | 279278 | N48590 | 0.43 | 2.19 | 5.11 | 1.25 | 13.11 | 0.98 | 1 |
| 1619 | 840786 | AA486084 | 1.09 | 5.54 | 5.10 | 0.66 | 13.43 | 1.23 | 1 |
| 1620 | 288936 | N62696 | 0.17 | 0.87 | 5.10 | 1.51 | 13.26 | 0.52 | 1 |
| 1621 | 742607 | AA400389 | 0.83 | 4.24 | 5.10 | 1.21 | 13.06 | 1.01 | 1 |
| 1622 | 279806 | N49109 | 0.43 | 2.18 | 5.07 | 1.55 | 12.47 | 1.21 | 1 |
| 1623 | 301842 | N91175 | 2.45 | 12.39 | 5.06 | 1.14 | 13.28 | 0.75 | 1 |
| 1624 | 796123 | AA460963 | 0.35 | 1.78 | 5.06 | 2.38 | 11.90 | 0.88 | 1 |
| 1625 | 283122 | N45223 | 0.85 | 4.31 | 5.06 | 0.60 | 13.49 | 1.08 | 1 |
| 1626 | 812293 | AA455096 | 0.45 | 2.25 | 5.05 | 2.60 | 10.93 | 1.62 | 1 |
| 1627 | 278572 | N66178 | 2.84 | 14.29 | 5.04 | 1.69 | 11.65 | 1.78 | 1 |
| 1628 | 257746 | N30597 | 0.82 | 4.11 | 5.02 | 2.55 | 11.42 | 1.09 | 1 |
| 1629 | 257779 | N26993 | 0.52 | 2.63 | 5.01 | 1.70 | 12.41 | 0.93 | 1 |
| 1630 | 362795 | AA018683 | 0.01 | 0.05 | 5.01 | 13.03 | 1.00 | 1.00 | 1 |
| 1631 | 290680 | N67619 | 0.55 | 2.76 | 5.00 | 1.21 | 11.91 | 1.89 | 1 |
| 1632 | 119289 | 798056 | 0.20 | 0.98 | 5.00 | 1.31 | 12.76 | 0.93 | 1 |
| 1633 | 285680 | N67570 | 1.14 | 5.69 | 4.99 | 0.73 | 13.08 | 1.15 | 1 |
| 1634 | 784130 | AA432075 | 1.61 | 7.99 | 4.97 | 13.44 | 0.13 | 1.33 | 1 |
| 1635 | 345118 | W72920 | 0.45 | 2.23 | 4.96 | 0.78 | 12.45 | 1.64 | 1 |
| 1636 | 428298 | AA004946 | 0.76 | 3.75 | 4.95 | 1.05 | 12.80 | 0.98 | 1 |
| 1637 | 813242 | AA455904 | 0.24 | 1.17 | 4.94 | 0.43 | 13.67 | 0.73 | 1 |
| 1638 | 232789 | H73947 | 5.50 | 27.18 | 4.94 | 0.96 | 2.40 | 11.45 | 1 |
| 1639 | 41558 | R67269 | 0.31 | 1.54 | 4.94 | 0.53 | 13.93 | 0.36 | 1 |
| 1540 | 589861 | AA156112 | 0.68 | 3.33 | 4.94 | 0.78 | 12.51 | 1.52 | 1 |
| 1641 | 346997 | W79445 | 2.28 | 11.24 | 4.93 | 0.21 | 14.40 | 0.20 | 1 |
| 1642 | 743058 | AA406083 | 0.50 | 2.47 | 4.93 | 1.19 | 12.16 | 1.44 | 1 |
| 1643 | 811899 | AA454652 | 2.37 | 11.09 | 4.93 | 1.24 | 11.25 | 2.29 | 1 |
| 1644 | 841022 | AA486864 | 1.30 | 6.40 | 4.92 | 1.30 | 12.51 | 0.96 | 1 |
| 1645 | 1466633 | AA883680 | 0.16 | 0.81 | 4.92 | 1.09 | 10.94 | 2.74 | 1 |
| 1646 | 648058 | AA206865 | 1.12 | 5.51 | 4.92 | 2.39 | 10.12 | 2.25 | 1 |
| 1647 | 272169 | N35489 | 0.19 | 0.95 | 4.91 | 0.82 | 13.48 | 0.44 | 1 |
| 1648 | 609047 | AA187500 | 0.68 | 3.35 | 4.91 | 1.14 | 12.92 | 0.66 | 1 |
| 1649 | 47114 | H11071 | 0.23 | 1.12 | 4.91 | 1.98 | 12.52 | 0.22 | 1 |
| 1650 | 731404 | AA412247 | 0.94 | 4.63 | 4.90 | 0.99 | 13.24 | 0.48 | 1 |
| 1651 | 490484 | AA101632 | 0.24 | 1.16 | 4.90 | 14.47 | 0.18 | 0.05 | 1 |
| 1652 | 49203 | H15695 | 0.24 | 1.19 | 4.89 | 1.09 | 1.08 | 12.48 | 1 |
| 1653 | 280763 | N50563 | 0.04 | 0.19 | 4.86 | 1.56 | 12.12 | 0.96 | 1 |
| 1654 | 731393 | AA421054 | 0.27 | 1.31 | 4.88 | 1.60 | 11.90 | 1.14 | 1 |
| 1655 | 430276 | AA010388 | 1.14 | 5.56 | 4.88 | 1.59 | 12.60 | 0.45 | 1 |
| 1656 | 768008 | AA418744 | 1.40 | 6.82 | 4.87 | 2.38 | 0.46 | 11.76 | 1 |
| 1657 | 589484 | AA146826 | 0.37 | 1.82 | 4.86 | 1.34 | 11.70 | 1.55 | 1 |
| 1658 | 488683 | AA044903 | 1.84 | 8.97 | 4.86 | 0.79 | 12.99 | 0.81 | 1 |
| 1659 | 322079 | W37753 | 2.68 | 13.01 | 4.86 | 1.09 | 11.94 | 1.55 | 1 |
| 1660 | 214006 | H70775 | 0.13 | 0.65 | 4.86 | 1.07 | 11.25 | 2.25 | 1 |
| 1661 | 166245 | R88267 | 0.01 | 0.05 | 4.86 | 1.00 | 1.00 | 12.57 | 1 |
| 1662 | 36688 | R62470 | 0.43 | 2.06 | 4.85 | 1.10 | 12.19 | 1.25 | 1 |
| 1663 | 115277 | T86932 | 0.64 | 3.08 | 4.84 | 1.16 | 12.34 | 1.04 | 1 |
| 1664 | 167076 | R89700 | 1.77 | 8.56 | 4.84 | 1.76 | 1.07 | 11.69 | 1 |
| 1665 | 289928 | N64603 | 0.64 | 3.08 | 4.84 | 1.04 | 11.69 | 1.78 | 1 |
| 1666 | 731205 | AA417275 | 0.08 | 0.36 | 4.83 | 1.64 | 12.71 | 0.13 | 1 |
| 1667 | 242037 | H93328 | 0.42 | 2.05 | 4.83 | 0.83 | 1.93 | 11.72 | 1 |
| 1668 | 415976 | W85709 | 0.67 | 3.23 | 4.82 | 1.36 | 11.63 | 1.48 | 1 |
| 1669 | 509564 | AA045574 | 2.68 | 12.92 | 4.82 | 1.96 | 11.76 | 0.74 | 1 |
| 1670 | 824358 | AA489681 | 0.49 | 2.35 | 4.82 | 0.97 | 11.57 | 1.92 | 1 |
| 1671 | 743416 | AA609334 | 1.12 | 5.41 | 4.82 | 0.99 | 12.76 | 0.71 | 1 |
| 1672 | 289666 | N77779 | 0.04 | 0.21 | 4.82 | 1.07 | 11.91 | 1.48 | 1 |
| 1673 | 1034738 | AA780190 | 3.28 | 15.82 | 4.82 | 10.78 | 1.98 | 1.68 | 1 |
| 1674 | 796199 | AA461119 | 0.82 | 3.96 | 4.81 | 0.92 | 12.21 | 1.32 | 1 |
| 1675 | 137017 | R35665 | 0.01 | 0.04 | 4.79 | 11.66 | 1.36 | 1.36 | 1 |
| 1676 | 163174 | H27379 | 5.35 | 25.60 | 4.79 | 1.89 | 1.32 | 11.14 | 1 |
| 1677 | 898195 | AA598587 | 1.46 | 6.99 | 4.78 | 2.18 | 0.33 | 11.85 | 1 |
| 1678 | 295483 | W05026 | 54.50 | 260.67 | 4.78 | 1.67 | 1.75 | 10.93 | 1 |
| 1679 | 429202 | AA005358 | 0.42 | 2.01 | 4.78 | 1.06 | 12.91 | 0.38 | 1 |
| 1680 | 703581 | AA278759 | 4.41 | 21.07 | 4.77 | 0.10 | 13.94 | 0.29 | 1 |
| 1681 | 377051 | AA057620 | 0.14 | 0.65 | 4.77 | 0.47 | 12.05 | 1.79 | 1 |
| 1682 | 1390860 | AA844447 | 1.17 | 5.60 | 4.77 | 0.94 | 12.67 | 0.71 | 1 |
| 1683 | 743536 | AA609422 | 0.07 | 0.31 | 4.75 | 2.16 | 10.53 | 1.56 | 1 |
| 1684 | 795810 | AA459857 | 0.72 | 3.40 | 4.75 | 0.91 | 12.47 | 0.86 | 1 |
| 1685 | 858761 | AA779048 | 0.73 | 3.45 | 4.74 | 0.47 | 13.16 | 0.60 | 1 |
| 1686 | 279771 | N49098 | 0.92 | 4.38 | 4.74 | 1.77 | 11.33 | 1.12 | 1 |
| 1687 | 289794 | N59295 | 1.45 | 6.86 | 4.73 | 0.70 | 11.93 | 1.55 | 1 |
| 1688 | 297857 | N68928 | 0.28 | 1.32 | 4.72 | 0.64 | 12.95 | 0.57 | 1 |
| 1689 | 743901 | AA634482 | 0.43 | 2.04 | 4.71 | 1.57 | 11.28 | 1.27 | 1 |
| 1690 | 788519 | AA452578 | 1.20 | 5.63 | 4.71 | 1.23 | 12.29 | 0.60 | 1 |
| 1691 | 811907 | AA454654 | 0.10 | 0.47 | 4.71 | 1.82 | 11.88 | 0.42 | 1 |
| 1692 | 796170 | AA461086 | 0.83 | 3.89 | 4.70 | 0.87 | 12.35 | 0.88 | 1 |
| 1693 | 782208 | AA431975 | 0.68 | 3.20 | 4.69 | 1.26 | 11.65 | 1.16 | 1 |
| 1694 | 256907 | N30096 | 0.58 | 2.71 | 4.68 | 12.24 | 0.73 | 1.07 | 1 |
| 1695 | 327480 | W20462 | 0.20 | 0.92 | 4.68 | 1.83 | 11.61 | 0.60 | 1 |
| 1896 | 306513 | N91811 | 1.15 | 5.40 | 4.68 | 0.40 | 1.53 | 12.09 | 1 |
| 1697 | 665148 | AA195651 | 1.11 | 5.18 | 4.67 | 0.54 | 12.86 | 0.62 | 1 |
| 1698 | 43936 | H05777 | 0.64 | 3.00 | 4.67 | 1.66 | 11.17 | 1.17 | 1 |
| 1699 | 429681 | AA011597 | 1.17 | 5.43 | 4.65 | 0.88 | 12.67 | 0.41 | 1 |
| 1700 | 752770 | AA417895 | 2.01 | 9.34 | 4.65 | 2.12 | 0.35 | 11.48 | 1 |
| 1701 | 22541 | T89084 | 0.18 | 0.82 | 4.61 | 0.90 | 11.08 | 1.84 | 1 |
| 1702 | 277063 | N39590 | 1.09 | 5.04 | 4.61 | 1.05 | 11.59 | 1.18 | 1 |
| 1703 | 743193 | AA401436 | 0.59 | 2.73 | 4.60 | 1.39 | 11.34 | 1.08 | 1 |
| 1704 | 1161564 | AA877815 | 1.06 | 4.90 | 4.60 | 1.72 | 1.15 | 10.93 | 1 |
| 1705 | 842927 | AA486445 | 2.04 | 9.37 | 4.60 | 1.65 | 11.22 | 0.94 | 1 |
| 1706 | 1476065 | AA873060 | 22.57 | 103.69 | 4.59 | 1.67 | 0.01 | 12.11 | 1 |
| 1707 | 730343 | AA412485 | 0.38 | 1.74 | 4.58 | 1.32 | 11.01 | 1.41 | 1 |
| 1708 | 345318 | W73022 | 0.30 | 1.38 | 4.58 | 1.45 | 11.30 | 1.00 | 1 |
| 1709 | 306243 | N90583 | 0.84 | 3.82 | 4.56 | 0.97 | 11.01 | 1.70 | 1 |
| 1710 | 743531 | AA609430 | 0.50 | 2.28 | 4.55 | 1.53 | 11.06 | 1.05 | 1 |
| 1711 | 251212 | H97514 | 0.65 | 2.94 | 4.54 | 0.98 | 11.38 | 1.26 | 1 |
| 1712 | 809976 | AA454844 | 0.82 | 3.70 | 4.54 | 0.84 | 11.52 | 1.25 | 1 |
| 1713 | 418400 | W92798 | 0.88 | 4.00 | 4.54 | 0.79 | 12.54 | 0.28 | 1 |
| 1714 | 448024 | AA702788 | 0.99 | 4.48 | 4.53 | 1.05 | 12.10 | 0.45 | 1 |
| 1715 | 841153 | AA487046 | 0.90 | 4.06 | 4.53 | 1.23 | 11.03 | 1.34 | 1 |
| 1716 | 434828 | AA703117 | 0.99 | 4.46 | 4.51 | 1.04 | 0.90 | 11.60 | 1 |
| 1717 | 50781 | H17022 | 0.37 | 1.66 | 4.51 | 10.45 | 0.97 | 2.11 | 1 |
| 1718 | 842847 | AA486288 | 0.66 | 2.99 | 4.51 | 0.52 | 10.76 | 2.26 | 1 |
| 1719 | 743893 | AA634479 | 0.31 | 1.40 | 4.51 | 1.26 | 10.02 | 2.26 | 1 |
| 1720 | 788246 | AA454085 | 0.84 | 3.80 | 4.51 | 0.88 | 12.14 | 0.51 | 1 |
| 1721 | 726663 | AA398247 | 0.45 | 2.04 | 4.51 | 1.01 | 12.07 | 0.44 | 1 |
| 1722 | 36491 | R48700 | 0.13 | 0.60 | 4.50 | 0.90 | 11.61 | 0.98 | 1 |
| 1723 | 347066 | W79525 | 0.45 | 2.03 | 4.49 | 1.24 | 10.60 | 1.64 | 1 |
| 1724 | 859382 | AA666087 | 0.48 | 2.17 | 4.49 | 0.93 | 11.03 | 1.60 | 1 |
| 1725 | 489549 | AA101833 | 0.72 | 3.23 | 4.49 | 1.68 | 10.50 | 1.29 | 1 |
| 1726 | 254468 | N22210 | 1.01 | 4.55 | 4.49 | 1.12 | 11.58 | 0.76 | 1 |
| 1727 | 293013 | N63768 | 0.01 | 0.04 | 4.49 | 1.00 | 1.00 | 11.46 | 1 |
| 1728 | 416889 | W86832 | 1.38 | 6.20 | 4.48 | 1.04 | 12.03 | 0.39 | 1 |
| 1729 | 789383 | AA484861 | 1.80 | 8.04 | 4.46 | 1.80 | 10.20 | 1.39 | 1 |
| 1730 | 431255 | AA682549 | 0.58 | 2.59 | 4.45 | 0.58 | 12.07 | 0.71 | 1 |
| 1731 | 377898 | AA777098 | 0.64 | 2.85 | 4.45 | 11.75 | 0.61 | 1.00 | 1 |
| 1732 | 200656 | R98407 | 0.52 | 2.32 | 4.45 | 0.86 | 10.04 | 2.45 | 1 |
| 1733 | 280431 | N51574 | 0.69 | 3.08 | 4.44 | 0.91 | 11.55 | 0.87 | 1 |
| 1734 | 48060 | H11631 | 0.39 | 1.74 | 4.44 | 0.91 | 11.78 | 0.63 | 1 |
| 1735 | 75644 | T58430 | 0.15 | 0.64 | 4.43 | 0.82 | 10.29 | 2.19 | 1 |
| 1736 | 795349 | AA453261 | 0.43 | 1.90 | 4.43 | 1.34 | 10.92 | 1.04 | 1 |
| 1737 | 284803 | N59870 | 0.42 | 1.88 | 4.43 | 1.21 | 11.30 | 0.77 | 1 |
| 1738 | 488276 | AA085759 | 0.25 | 1.11 | 4.42 | 1.34 | 10.60 | 1.31 | 1 |
| 1739 | 730147 | AA412499 | 1.04 | 4.58 | 4.42 | 1.10 | 11.12 | 1.03 | 1 |
| 1740 | 773512 | AA427947 | 0.93 | 4.12 | 4.42 | 0.49 | 12.55 | 0.21 | 1 |
| 1741 | 24895 | R39014 | 0.28 | 1.25 | 4.41 | 0.88 | 11.25 | 1.12 | 1 |
| 1742 | 130742 | R22024 | 1.19 | 5.27 | 4.41 | 0.89 | 11.45 | 0.90 | 1 |
| 1743 | 259374 | N31952 | 1.42 | 6.26 | 4.41 | 12.36 | 0.39 | 0.48 | 1 |
| 1744 | 362279 | AA001222 | 0.05 | 0.21 | 4.41 | 2.15 | 0.21 | 10.87 | 1 |
| 1745 | 134666 | R28254 | 1.25 | 5.48 | 4.40 | 0.94 | 1.71 | 10.55 | 1 |
| 1746 | 252515 | H87471 | 4.33 | 18.96 | 4.38 | 0.21 | 0.18 | 12.75 | 1 |
| 1747 | 50986 | H18433 | 0.09 | 0.38 | 4.38 | 2.06 | 0.11 | 10.97 | 1 |
| 1748 | 595604 | AA167269 | 3.85 | 16.86 | 4.38 | 2.12 | 0.08 | 10.93 | 1 |
| 1749 | 1049321 | AA620783 | 0.16 | 0.68 | 4.37 | 0.98 | 11.12 | 1.00 | 1 |
| 1750 | 201045 | R99831 | 0.19 | 0.83 | 4.37 | 0.99 | 10.89 | 1.23 | 1 |
| 1751 | 379709 | AA778089 | 0.47 | 2.03 | 4.35 | 10.31 | 1.12 | 1.63 | 1 |
| 1752 | 625838 | AA187679 | 0.93 | 4.05 | 4.35 | 1.46 | 10.22 | 1.37 | 1 |
| 1753 | 594556 | AA169121 | 1.83 | 7.94 | 4.35 | 1.39 | 10.88 | 0.78 | 1 |
| 1754 | 1031367 | AA609135 | 0.55 | 2.37 | 4.35 | 1.11 | 11.31 | 0.62 | 1 |
| 1755 | 462598 | AA705103 | 0.24 | 1.03 | 4.34 | 1.13 | 10.36 | 1.54 | 1 |
| 1756 | 1031907 | AA609744 | 0.81 | 3.50 | 4.34 | 1.33 | 10.40 | 1.29 | 1 |
| 1757 | 811764 | AA463444 | 0.17 | 0.73 | 4.33 | 0.81 | 11.87 | 0.31 | 1 |
| 1758 | 278198 | N83543 | 0.49 | 2.12 | 4.33 | 1.18 | 11.07 | 0.73 | 1 |
| 1759 | 1417934 | AA878731 | 0.66 | 2.84 | 4.32 | 0.63 | 11.68 | 0.65 | 1 |
| 1760 | 30821 | R42569 | 0.29 | 1.27 | 4.32 | 0.99 | 10.57 | 1.39 | 1 |
| 1761 | 35410 | R43805 | 0.15 | 0.65 | 4.29 | 1.09 | 10.98 | 0.82 | 1 |
| 1762 | 796711 | AA460702 | 0.56 | 2.39 | 4.29 | 1.04 | 10.85 | 0.97 | 1 |
| 1763 | 838278 | AA457474 | 0.55 | 2.36 | 4.28 | 0.87 | 10.98 | 0.97 | 1 |
| 1764 | 782773 | AA448172 | 1.25 | 5.35 | 4.27 | 1.36 | 10.44 | 1.02 | 1 |
| 1765 | 418384 | W92970 | 1.03 | 4.38 | 4.27 | 1.51 | 10.60 | 0.69 | 1 |
| 1766 | 254276 | N22495 | 1.10 | 4.69 | 4.26 | 1.04 | 10.91 | 0.84 | 1 |
| 1767 | 731183 | AA417250 | 1.41 | 8.00 | 4.26 | 0.65 | 11.45 | 0.68 | 1 |
| 1768 | 767284 | AA418414 | 0.66 | 2.81 | 4.25 | 0.92 | 11.09 | 0.76 | 1 |
| 1769 | 813639 | AA447743 | 0.60 | 2.56 | 4.25 | 1.83 | 10.13 | 0.79 | 1 |
| 1770 | 276689 | N34892 | 0.65 | 2.74 | 4.24 | 1.02 | 10.76 | 0.96 | 1 |
| 1771 | 726709 | AA398267 | 0.02 | 0.10 | 4.24 | 1.53 | 10.89 | 0.30 | 1 |
| 1772 | 81427 | T60168 | 1.10 | 4.66 | 4.23 | 0.41 | 0.63 | 11.65 | 1 |
| 1773 | 271672 | N31564 | 2.82 | 11.90 | 4.21 | 0.82 | 0.72 | 11.10 | 1 |
| 1774 | 30894 | R41804 | 0.20 | 0.86 | 4.21 | 1.17 | 10.21 | 1.25 | 1 |
| 1775 | 306005 | N91382 | 0.47 | 1.98 | 4.21 | 1.25 | 10.85 | 0.52 | 1 |
| 1776 | 280453 | N51583 | 0.77 | 3.22 | 4.21 | 1.14 | 10.41 | 1.07 | 1 |
| 1777 | 782217 | AA431988 | 0.66 | 2.79 | 4.20 | 11.47 | 0.38 | 0.75 | 1 |
| 1778 | 795452 | AA454186 | 0.65 | 2.71 | 4.20 | 1.18 | 10.46 | 0.96 | 1 |
| 1779 | 795168 | AA453465 | 0.83 | 3.49 | 4.20 | 1.08 | 10.99 | 0.52 | 1 |
| 1780 | 308066 | N91003 | 1.33 | 5.55 | 4.19 | 11.10 | 0.35 | 1.12 | 1 |
| 1781 | 730100 | AA412446 | 0.42 | 1.75 | 4.19 | 1.10 | 10.39 | 1.07 | 1 |
| 1782 | 280217 | N47941 | 1.55 | 6.49 | 4.19 | 11.57 | 0.51 | 0.49 | 1 |
| 1783 | 415145 | W95082 | 0.64 | 2.67 | 4.19 | 11.74 | 0.02 | 0.81 | 1 |
| 1784 | 1056172 | AA620995 | 9.21 | 38.53 | 4.19 | 0.25 | 0.15 | 12.16 | 1 |
| 1785 | 731298 | AA421001 | 0.87 | 3.65 | 4.18 | 1.12 | 10.52 | 0.91 | 1 |
| 1786 | 415413 | W81117 | 1.06 | 4.43 | 4.18 | 1.35 | 10.31 | 0.89 | 1 |
| 1787 | 785760 | AA449686 | 0.08 | 0.35 | 4.17 | 0.54 | 11.10 | 0.88 | 1 |
| 1788 | 809863 | AA455130 | 0.75 | 3.11 | 4.13 | 1.41 | 10.02 | 0.97 | 1 |
| 1789 | 730406 | AA470079 | 1.07 | 4.41 | 4.13 | 1.25 | 10.33 | 0.80 | 1 |
| 1790 | 78647 | T50995 | 0.01 | 0.04 | 4.11 | 0.93 | 1.00 | 10.41 | 1 |
| 1791 | 1048872 | AA778533 | 0.25 | 1.03 | 4.10 | 0.41 | 10.14 | 1.75 | 1 |
| 1792 | 347268 | W80791 | 0.68 | 2.80 | 4.10 | 0.47 | 10.78 | 1.05 | 1 |
| 1793 | 812222 | AA455358 | 0.51 | 2.10 | 4.10 | 0.89 | 10.06 | 1.35 | 1 |
| 1794 | 412983 | AA707753 | 0.67 | 2.74 | 4.09 | 0.89 | 10.57 | 0.82 | 1 |
| 1795 | 29397 | R05660 | 0.66 | 2.71 | 4.09 | 0.93 | 10.58 | 0.76 | 1 |
| 1796 | 773392 | AA425749 | 0.35 | 1.43 | 4.09 | 0.95 | 10.32 | 0.99 | 1 |
| 1797 | 772916 | AA479912 | 0.03 | 0.13 | 4.07 | 1.43 | 10.47 | 0.31 | 1 |
| 1798 | 172785 | H20046 | 0.52 | 2.12 | 4.07 | 0.85 | 1.18 | 10.18 | 1 |
| 1799 | 782617 | AA447561 | 1.53 | 6.21 | 4.07 | 1.60 | 10.08 | 0.53 | 1 |
| 1800 | 841253 | AA487146 | 2.04 | 8.26 | 4.06 | 1.93 | 0.02 | 10.22 | 1 |
| 1801 | 725365 | AA292054 | 0.78 | 3.16 | 4.06 | 0.28 | 11.66 | 0.23 | 1 |
| 1802 | 625465 | AA187287 | 3.49 | 14.17 | 4.06 | 0.52 | 10.38 | 1.26 | 1 |
| 1803 | 435573 | AA701933 | 0.96 | 3.89 | 4.04 | 0.59 | 0.27 | 11.28 | 1 |
| 1804 | 53122 | R15891 | 0.76 | 3.07 | 4.04 | 0.82 | 11.02 | 0.28 | 1 |
| 1805 | 784064 | AA443823 | 1.53 | 6.18 | 4.04 | 0.96 | 10.39 | 0.76 | 1 |
| 1806 | 729924 | AA399633 | 0.44 | 1.77 | 4.02 | 0.96 | 10.21 | 0.91 | 1 |
| 1807 | 45542 | 808560 | 0.27 | 1.08 | 4.02 | 0.75 | 10.27 | 1.03 | 1 |
| 1808 | 1055278 | AA621478 | 0.22 | 0.90 | 4.01 | 0.79 | 10.00 | 1.25 | 1 |
| 1809 | 490071 | AA138071 | 0.50 | 2.00 | 4.01 | 1.16 | 10.15 | 0.72 | 1 |
| 1810 | 300024 | N78903 | 2.24 | 8.94 | 4.00 | 0.93 | 10.51 | 0.56 | 1 |
| 1811 | 665738 | AA194189 | 0.29 | 1.14 | 3.98 | 0.96 | 10.42 | 0.55 | 1 |
| 1812 | 27072 | R37780 | 0.56 | 2.21 | 3.96 | 0.80 | 10.16 | 0.91 | 1 |
| 1813 | 593218 | AA159613 | 1.57 | 6.19 | 3.95 | 0.80 | 10.43 | 0.62 | 1 |
| 1814 | 491690 | AA150401 | 0.63 | 2.48 | 3.94 | 0.76 | 10.07 | 1.00 | 1 |
| 1815 | 772904 | AA479906 | 0.01 | 0.05 | 3.94 | 10.25 | 0.79 | 0.79 | 1 |
| 1816 | 784105 | AA432064 | 0.25 | 0.98 | 3.94 | 0.46 | 11.14 | 0.22 | 1 |
| 1817 | 1031279 | AA609056 | 0.85 | 3.36 | 3.94 | 0.87 | 10.25 | 0.68 | 1 |
| 1818 | 754333 | AA479313 | 2.55 | 9.98 | 3.91 | 0.74 | 0.95 | 10.05 | 1 |
| 1819 | 45783 | H08598 | 0.20 | 0.77 | 3.91 | 1.30 | 10.29 | 0.14 | 1 |
| 1820 | 1455671 | AA863169 | 2.69 | 10.36 | 3.86 | 0.88 | 10.27 | 0.43 | 1 |
| 1821 | 42115 | R60717 | 0.24 | 0.92 | 3.84 | 0.68 | 10.18 | 0.67 | 1 |
| 1822 | 795736 | AA460285 | 0.19 | 0.74 | 3.84 | 0.78 | 0.61 | 10.13 | 1 |
| 1823 | 449438 | AA777886 | 0.05 | 0.19 | 3.81 | 0.79 | 0.20 | 10.44 | 1 |
| 1824 | 757238 | AA426067 | 0.24 | 0.93 | 3.81 | 0.42 | 10.26 | 0.74 | 1 |
| 1825 | 41903 | R60705 | 0.11 | 0.42 | 3.78 | 0.25 | 10.60 | 0.48 | 1 |
| 1826 | 1466416 | AA885470 | 1.08 | 4.06 | 3.76 | 0.56 | 10.08 | 0.64 | 1 |
| 1827 | 744944 | AA625890 | 6.57 | 24.47 | 3.72 | 10.66 | 0.12 | 0.40 | 1 |
| 1828 | 609155 | AA176867 | 0.47 | 1.73 | 3.70 | 10.29 | 0.80 | 0.02 | 1 |
| 1829 | 429424 | AA007687 | 0.04 | 0.15 | 3.65 | 0.65 | 10.04 | 0.25 | 1 |
| 1830 | 504761 | AA148735 | 0.03 | 0.11 | 3.60 | 0.32 | 10.14 | 0.32 | 1 |

TABLE 2-11
Prostate Cancer Cell Lines vs. Normal Prostatic Epithelial Cell Line: Genes
upregulated 5 fold or more in at least 2 cancer cell lines.

| | | Gen Bank | | Ave Cancer | Fold(Ave | Fold | Fold | Fold | |
|---|---|---|---|---|---|---|---|---|---|
| | IMAGE | Accession | MPM1 | Cell Lines | Cell line/ | (MPM2/MP | (MPM271/M | (MPM272/M | # of Cancer |
| Order | Clone ID | Number | (PrEC) | Values | MPM1) | M1) | PM1) | PM1) | Cell lines |
| 1 | 757165 | AA443950 | 0.01 | 5.71 | 591.67 | 7.33 | 66.37 | 1701.31 | 3 |
| 2 | 592707 | AA160606 | 0.01 | 1.66 | 234.30 | 8.10 | 680.66 | 14.13 | 3 |
| 3 | 220851 | H95633 | 0.01 | 1.28 | 196.56 | 8.10 | 570.75 | 10.82 | 3 |
| 4 | 782804 | AA448186 | 0.01 | 1.81 | 180.70 | 8.88 | 526.08 | 7.15 | 3 |
| 5 | 1343732 | AA725564 | 0.01 | 1.12 | 112.48 | 13.45 | 301.13 | 22.85 | 3 |
| 6 | 784296 | AA447079 | 0.01 | 0.66 | 65.30 | 45.84 | 106.60 | 43.45 | 3 |
| 7 | 796569 | AA460463 | 0.01 | 0.35 | 63.57 | 16.10 | 138.91 | 35.70 | 3 |
| 8 | 1558655 | AA976561 | 0.08 | 4.36 | 54.32 | 97.06 | 17.92 | 47.97 | 3 |
| 9 | 743016 | AA406036 | 0.01 | 0.50 | 50.47 | 15.70 | 123.80 | 11.93 | 3 |
| 10 | 39442 | R51617 | 0.10 | 5.02 | 49.93 | 99.52 | 44.77 | 5.49 | 3 |
| 11 | 33076 | R44048 | 0.80 | 36.21 | 45.35 | 56.36 | 29.19 | 50.51 | 3 |
| 12 | 626390 | AA189106 | 0.07 | 3.16 | 45.16 | 91.62 | 20.97 | 22.90 | 3 |
| 13 | 128083 | R09747 | 0.01 | 0.44 | 44.34 | 25.03 | 101.07 | 6.93 | 3 |
| 14 | 812170 | AA456035 | 0.01 | 0.30 | 37.18 | 16.25 | 80.79 | 14.51 | 3 |
| 15 | 50288 | H17888 | 0.01 | 0.32 | 36.79 | 12.25 | 87.45 | 10.68 | 3 |
| 16 | 243181 | H94482 | 0.01 | 0.36 | 35.62 | 5.91 | 88.28 | 12.68 | 3 |
| 17 | 256911 | N30098 | 0.01 | 0.25 | 34.44 | 20.19 | 73.72 | 9.41 | 3 |
| 18 | 1030613 | AA608832 | 0.01 | 0.33 | 33.18 | 5.81 | 82.72 | 11.01 | 3 |
| 19 | 487861 | AA045436 | 0.02 | 0.59 | 32.57 | 29.44 | 39.44 | 28.82 | 3 |
| 20 | 270626 | N33331 | 0.01 | 0.32 | 31.52 | 8.48 | 9.21 | 76.87 | 3 |
| 21 | 291057 | N72115 | 0.23 | 6.85 | 29.19 | 24.48 | 38.30 | 24.80 | 3 |
| 22 | 1455835 | AA863292 | 0.01 | 0.40 | 29.16 | 9.10 | 30.24 | 48.14 | 3 |
| 23 | 108378 | T77729 | 0.01 | 0.41 | 29.13 | 7.73 | 36.47 | 43.19 | 3 |
| 24 | 1391682 | AA789328 | 0.02 | 0.44 | 28.83 | 5.99 | 65.78 | 14.72 | 3 |
| 25 | 263014 | H99813 | 0.63 | 17.34 | 27.31 | 19.07 | 21.59 | 41.29 | 3 |
| 26 | 303180 | N92764 | 0.01 | 0.27 | 27.21 | 13.82 | 36.04 | 31.77 | 3 |
| 27 | 42415 | R60981 | 0.12 | 3.11 | 26.71 | 9.46 | 65.35 | 5.30 | 3 |
| 28 | 768056 | AA418903 | 0.01 | 0.16 | 26.24 | 7.36 | 35.03 | 36.33 | 3 |
| 29 | 46896 | H09818 | 0.05 | 1.21 | 25.96 | 33.86 | 30.16 | 13.87 | 3 |
| 30 | 151184 | H02294 | 0.48 | 12.53 | 25.93 | 38.89 | 13.26 | 25.64 | 3 |
| 31 | 38344 | R49555 | 0.01 | 0.24 | 25.84 | 5.18 | 58.23 | 14.11 | 3 |
| 32 | 41595 | R59556 | 0.01 | 0.17 | 25.59 | 12.08 | 52.20 | 12.48 | 3 |
| 33 | 430092 | AA009840 | 0.40 | 10.00 | 24.80 | 34.18 | 13.61 | 26.60 | 3 |
| 34 | 1049033 | AA778675 | 0.38 | 9.30 | 24.51 | 46.38 | 16.26 | 10.88 | 3 |
| 35 | 884790 | AA629838 | 0.01 | 0.24 | 24.29 | 30.26 | 10.09 | 32.50 | 3 |
| 36 | 23345 | R39191 | 0.01 | 0.16 | 23.97 | 44.37 | 17.55 | 9.98 | 3 |
| 37 | 284714 | N63057 | 0.01 | 0.16 | 23.01 | 8.11 | 34.50 | 26.43 | 3 |
| 38 | 365149 | AA025142 | 0.16 | 3.52 | 22.24 | 38.20 | 16.96 | 11.55 | 3 |
| 39 | 418094 | W90036 | 0.01 | 0.21 | 20.90 | 5.07 | 7.89 | 49.72 | 3 |
| 40 | 51581 | H22824 | 0.01 | 0.25 | 20.42 | 18.18 | 18.45 | 24.62 | 3 |
| 41 | 270975 | N32542 | 0.01 | 0.20 | 20.40 | 5.36 | 33.42 | 22.42 | 3 |
| 42 | 51916 | H22563 | 0.12 | 2.46 | 20.27 | 20.94 | 30.57 | 9.30 | 3 |
| 43 | 40010 | R54036 | 0.01 | 0.19 | 19.50 | 7.04 | 11.18 | 40.27 | 3 |
| 44 | 1467161 | AA883187 | 0.02 | 0.31 | 18.24 | 10.20 | 24.35 | 20.16 | 3 |
| 45 | 488431 | AA047441 | 0.17 | 2.95 | 17.83 | 10.45 | 28.90 | 14.13 | 3 |
| 46 | 435099 | AA701315 | 0.01 | 0.21 | 17.36 | 7.52 | 33.82 | 10.73 | 3 |
| 47 | 67440 | T49355 | 1.96 | 33.78 | 17.25 | 25.95 | 8.91 | 16.88 | 3 |
| 48 | 280155 | N47012 | 0.04 | 0.63 | 17.15 | 5.64 | 11.78 | 34.04 | 3 |
| 49 | 454083 | AA676998 | 0.01 | 0.17 | 16.89 | 8.16 | 10.67 | 31.84 | 3 |
| 50 | 347586 | W81432 | 0.01 | 0.17 | 16.56 | 7.84 | 8.92 | 32.94 | 3 |
| 51 | 269606 | N26769 | 0.01 | 0.21 | 16.54 | 6.72 | 36.98 | 5.91 | 3 |
| 52 | 358990 | W92263 | 0.01 | 0.16 | 16.35 | 10.47 | 5.09 | 33.48 | 3 |
| 53 | 235164 | H79466 | 0.01 | 0.09 | 16.34 | 9.38 | 30.25 | 9.40 | 3 |
| 54 | 357373 | W93717 | 0.70 | 11.31 | 16.17 | 5.81 | 15.89 | 26.82 | 3 |
| 55 | 82869 | T69270 | 0.02 | 0.32 | 15.94 | 21.48 | 14.07 | 12.25 | 3 |
| 56 | 767315 | AA418545 | 0.02 | 0.29 | 15.63 | 6.61 | 33.79 | 6.49 | 3 |
| 57 | 447416 | AA702335 | 0.25 | 3.80 | 15.00 | 33.97 | 5.66 | 5.36 | 3 |
| 58 | 250069 | H97140 | 0.02 | 0.35 | 14.92 | 5.14 | 24.61 | 15.00 | 3 |
| 59 | 28298 | R40434 | 0.02 | 0.35 | 14.61 | 5.11 | 16.46 | 22.27 | 3 |
| 60 | 757389 | AA437142 | 0.01 | 0.14 | 14.54 | 5.64 | 32.86 | 5.13 | 3 |
| 61 | 25915 | R39951 | 0.01 | 0.14 | 14.20 | 9.65 | 6.51 | 26.44 | 3 |
| 62 | 1031446 | AA609189 | 0.04 | 0.50 | 14.14 | 5.44 | 31.74 | 5.25 | 3 |
| 63 | 32109 | R42780 | 0.09 | 1.25 | 14.14 | 31.21 | 5.86 | 5.35 | 3 |
| 64 | 30850 | R42600 | 0.03 | 0.39 | 13.78 | 6.39 | 29.82 | 5.12 | 3 |
| 65 | 1035796 | AA628867 | 1.14 | 15.63 | 13.71 | 10.64 | 9.76 | 20.74 | 3 |
| 66 | 898286 | AA598974 | 1.21 | 16.52 | 13.65 | 13.47 | 11.86 | 15.62 | 3 |
| 67 | 502003 | AA128617 | 0.01 | 0.17 | 13.58 | 7.33 | 15.99 | 17.41 | 3 |
| 68 | 359269 | AA016225 | 0.01 | 0.13 | 13.43 | 9.24 | 8.92 | 22.12 | 3 |
| 69 | 415089 | W93379 | 0.65 | 8.63 | 13.24 | 8.23 | 12.04 | 19.45 | 3 |
| 70 | 1466409 | AA885478 | 0.14 | 1.91 | 13.23 | 22.16 | 9.42 | 8.12 | 3 |
| 71 | 51103 | H19217 | 0.02 | 0.23 | 13.14 | 14.72 | 17.85 | 6.86 | 3 |
| 72 | 244050 | N34042 | 0.01 | 0.17 | 12.88 | 5.65 | 21.72 | 11.26 | 3 |
| 73 | 897807 | AA598531 | 0.29 | 3.56 | 12.37 | 21.95 | 7.81 | 7.37 | 3 |
| 74 | 845441 | AA644550 | 0.01 | 0.12 | 12.30 | 5.26 | 7.58 | 24.06 | 3 |
| 75 | 686172 | AA262211 | 1.18 | 14.39 | 12.18 | 5.65 | 13.80 | 17.09 | 3 |
| 76 | 1030798 | AA609002 | 0.03 | 0.31 | 11.73 | 10.15 | 8.33 | 16.72 | 3 |
| 77 | 210820 | H67712 | 0.04 | 0.45 | 11.30 | 5.06 | 7.88 | 20.94 | 3 |
| 78 | 788209 | AA453433 | 0.01 | 0.09 | 10.70 | 11.07 | 8.10 | 12.92 | 3 |
| 79 | 204483 | H58234 | 1.27 | 13.51 | 10.64 | 11.48 | 13.18 | 7.26 | 3 |
| 80 | 430928 | AA678295 | 0.74 | 7.64 | 10.34 | 20.21 | 5.77 | 5.03 | 3 |
| 81 | 376452 | AA041197 | 0.03 | 0.32 | 9.78 | 5.69 | 8.56 | 15.10 | 3 |
| 82 | 433567 | AA701652 | 0.02 | 0.16 | 9.57 | 11.04 | 10.28 | 7.37 | 3 |
| 83 | 280375 | N47113 | 1.12 | 10.61 | 9.52 | 11.98 | 10.79 | 5.79 | 3 |
| 84 | 303099 | N90779 | 0.15 | 1.40 | 9.48 | 5.53 | 11.42 | 11.50 | 3 |
| 85 | 448059 | AA702684 | 0.22 | 2.04 | 9.46 | 6.47 | 14.40 | 7.50 | 3 |
| 86 | 269300 | N24042 | 1.12 | 10.53 | 9.44 | 5.48 | 13.22 | 9.62 | 3 |
| 87 | 489213 | AA045665 | 0.37 | 3.43 | 9.32 | 9.36 | 11.24 | 7.35 | 3 |
| 88 | 43733 | H04789 | 0.11 | 1.03 | 9.24 | 10.06 | 12.44 | 5.24 | 3 |
| 89 | 234617 | H77727 | 0.07 | 0.60 | 9.16 | 12.99 | 8.66 | 5.84 | 3 |
| 90 | 177884 | H46176 | 0.16 | 1.44 | 9.15 | 5.38 | 8.45 | 13.62 | 3 |
| 91 | 897956 | AA598817 | 0.15 | 1.40 | 9.13 | 13.21 | 8.47 | 5.71 | 3 |
| 92 | 324901 | W49672 | 0.01 | 0.11 | 8.92 | 7.38 | 12.47 | 6.90 | 3 |
| 93 | 78217 | T50699 | 0.01 | 0.05 | 8.82 | 9.14 | 7.21 | 10.11 | 3 |
| 94 | 624627 | AA187351 | 0.56 | 4.90 | 8.72 | 5.30 | 9.17 | 11.70 | 3 |
| 95 | 491184 | AA137072 | 1.49 | 12.90 | 8.67 | 6.69 | 13.78 | 5.53 | 3 |
| 96 | 826273 | AA520999 | 0.45 | 3.90 | 8.62 | 5.02 | 10.99 | 9.84 | 3 |
| 97 | 42906 | R60019 | 0.19 | 1.57 | 8.31 | 9.78 | 8.33 | 6.83 | 3 |
| 98 | 773383 | AA425755 | 0.94 | 7.83 | 8.30 | 5.27 | 11.10 | 8.51 | 3 |
| 99 | 813271 | AA455938 | 0.07 | 0.54 | 7.87 | 11.86 | 6.62 | 5.12 | 3 |
| 100 | 200402 | R96941 | 0.75 | 5.69 | 7.61 | 8.56 | 5.22 | 9.05 | 3 |
| 101 | 950690 | AA608568 | 0.73 | 5.53 | 7.61 | 7.77 | 7.09 | 7.97 | 3 |
| 102 | 260035 | N30372 | 0.02 | 0.15 | 7.55 | 6.04 | 5.10 | 11.50 | 3 |
| 103 | 52704 | H29227 | 0.53 | 3.90 | 7.31 | 7.30 | 6.65 | 7.97 | 3 |
| 104 | 740620 | AA477400 | 0.02 | 0.12 | 7.30 | 6.01 | 6.44 | 9.46 | 3 |
| 105 | 853368 | AA663310 | 2.51 | 18.03 | 7.18 | 5.15 | 7.63 | 8.78 | 3 |
| 106 | 126650 | R06944 | 0.43 | 3.05 | 7.05 | 5.35 | 7.83 | 7.98 | 3 |
| 107 | 433576 | AA701645 | 0.10 | 0.72 | 6.85 | 10.00 | 5.36 | 5.19 | 3 |
| 108 | 134719 | R28287 | 0.31 | 2.07 | 6.69 | 9.45 | 5.02 | 5.60 | 3 |
| 109 | 151055 | H02231 | 7.49 | 47.65 | 6.36 | 6.67 | 5.78 | 6.63 | 3 |
| 110 | 128947 | R10284 | 1.20 | 7.33 | 6.13 | 6.92 | 5.67 | 5.79 | 3 |
| 111 | 773073 | AA425302 | 2.73 | 16.66 | 6.10 | 7.47 | 5.73 | 5.08 | 3 |
| 112 | 814306 | AA459318 | 4.89 | 29.59 | 6.05 | 6.00 | 6.93 | 5.21 | 3 |
| 113 | 275871 | R93875 | 0.01 | 0.07 | 5.99 | 5.03 | 7.41 | 5.52 | 3 |
| 114 | 45882 | H08785 | 0.34 | 1.94 | 5.63 | 5.49 | 5.63 | 5.77 | 3 |
| 115 | 40299 | R52085 | 0.02 | 0.12 | 5.20 | 5.19 | 5.01 | 5.40 | 3 |
| 116 | 299559 | N74995 | 0.01 | 9.92 | 991.80 | 1.38 | 2966.06 | 7.96 | 2 |
| 117 | 365551 | AA009615 | 0.01 | 8.39 | 838.64 | 1.96 | 2506.90 | 7.05 | 2 |
| 118 | 42627 | R60995 | 0.01 | 9.14 | 660.46 | 2.32 | 61.14 | 1917.92 | 2 |
| 119 | 277327 | N57483 | 0.01 | 9.00 | 613.07 | 1.58 | 1831.96 | 5.67 | 2 |
| 120 | 773640 | AA433885 | 0.01 | 5.08 | 508.28 | 2.41 | 1516.35 | 6.10 | 2 |
| 121 | 290213 | N64379 | 0.01 | 3.94 | 393.88 | 0.73 | 1171.33 | 9.57 | 2 |
| 122 | 510631 | AA099431 | 0.01 | 3.19 | 319.36 | 4.57 | 944.19 | 9.33 | 2 |
| 123 | 781157 | AA429904 | 0.01 | 2.83 | 283.26 | 1.00 | 841.07 | 7.73 | 2 |
| 124 | 781036 | AA446446 | 0.01 | 1.70 | 228.86 | 10.58 | 674.66 | 1.35 | 2 |
| 125 | 768226 | AA424900 | 0.01 | 1.04 | 103.67 | 3.09 | 302.09 | 5.84 | 2 |
| 126 | 203878 | H56453 | 0.02 | 1.93 | 95.78 | 0.80 | 140.40 | 146.14 | 2 |
| 127 | 366039 | AA071503 | 0.03 | 2.66 | 86.03 | 5.01 | 249.63 | 3.45 | 2 |
| 128 | 815794 | AA485214 | 0.08 | 6.83 | 85.65 | 0.89 | 150.46 | 105.59 | 2 |
| 129 | 795627 | AA459917 | 0.01 | 0.51 | 85.17 | 3.09 | 239.88 | 12.54 | 2 |
| 130 | 347182 | W80611 | 0.01 | 0.82 | 81.58 | 1.42 | 229.57 | 13.76 | 2 |
| 131 | 1161830 | AA876021 | 0.01 | 0.71 | 71.26 | 3.19 | 203.18 | 7.41 | 2 |
| 132 | 746152 | AA419486 | 0.08 | 5.16 | 65.49 | 0.87 | 95.72 | 99.88 | 2 |
| 133 | 346942 | W94289 | 0.01 | 0.61 | 61.20 | 3.73 | 168.10 | 11.76 | 2 |
| 134 | 395708 | AA757754 | 0.01 | 0.83 | 60.41 | 6.88 | 173.62 | 0.73 | 2 |
| 135 | 795858 | AA461522 | 0.03 | 1.60 | 60.34 | 6.29 | 170.43 | 4.30 | 2 |
| 136 | 131452 | R23270 | 0.45 | 26.93 | 59.79 | 104.24 | 4.69 | 70.45 | 2 |
| 137 | 684879 | AA251784 | 0.05 | 2.87 | 58.88 | 1.03 | 51.92 | 123.70 | 2 |
| 138 | 344589 | W73144 | 0.22 | 12.41 | 56.02 | 78.84 | 88.50 | 0.72 | 2 |
| 139 | 562811 | AA086475 | 0.01 | 0.28 | 53.90 | 5.36 | 154.39 | 1.93 | 2 |
| 140 | 30207 | R40231 | 0.04 | 2.21 | 52.11 | 0.54 | 150.40 | 5.39 | 2 |
| 141 | 757435 | AA437224 | 1.20 | 60.94 | 50.82 | 144.61 | 1.37 | 6.47 | 2 |
| 142 | 33827 | R44741 | 0.02 | 1.10 | 50.72 | 0.63 | 139.36 | 12.17 | 2 |
| 143 | 825740 | AA504844 | 0.26 | 13.02 | 49.67 | 0.91 | 56.58 | 91.51 | 2 |
| 144 | 785849 | AA449120 | 0.01 | 0.60 | 45.99 | 2.24 | 128.30 | 7.45 | 2 |
| 145 | 1389018 | AA855158 | 0.02 | 0.81 | 43.48 | 3.54 | 120.65 | 6.23 | 2 |
| 146 | 28498 | R37395 | 0.01 | 0.43 | 43.37 | 5.62 | 121.33 | 3.16 | 2 |
| 147 | 757368 | AA437126 | 0.01 | 0.42 | 41.72 | 4.59 | 109.53 | 11.05 | 2 |
| 148 | 1367900 | AA810225 | 0.01 | 0.41 | 41.11 | 4.81 | 99.20 | 19.33 | 2 |
| 149 | 366209 | AA062985 | 0.01 | 0.38 | 38.18 | 3.08 | 106.34 | 5.11 | 2 |
| 150 | 1435339 | AA857748 | 0.01 | 0.38 | 37.79 | 5.72 | 105.49 | 2.18 | 2 |
| 151 | 196544 | R91566 | 0.25 | 8.56 | 34.73 | 0.93 | 52.03 | 51.22 | 2 |
| 152 | 37823 | R59473 | 0.03 | 1.12 | 32.46 | 1.79 | 89.38 | 6.23 | 2 |
| 153 | 49858 | H29290 | 0.08 | 2.62 | 31.22 | 87.92 | 0.12 | 5.61 | 2 |
| 154 | 743189 | AA401434 | 0.02 | 0.70 | 30.56 | 2.89 | 81.20 | 7.60 | 2 |
| 155 | 815861 | AA485052 | 0.26 | 7.89 | 30.35 | 0.77 | 34.54 | 55.73 | 2 |
| 156 | 489656 | AA099369 | 0.01 | 0.30 | 30.08 | 1.00 | 7.97 | 81.28 | 2 |
| 157 | 38347 | R49439 | 0.21 | 5.88 | 27.65 | 1.51 | 76.38 | 5.06 | 2 |
| 158 | 167205 | R90934 | 0.58 | 16.16 | 27.63 | 72.37 | 9.84 | 0.70 | 2 |
| 159 | 53039 | R15740 | 0.01 | 0.27 | 27.34 | 0.74 | 70.25 | 11.03 | 2 |
| 160 | 796549 | AA460274 | 0.01 | 0.27 | 27.13 | 1.21 | 69.04 | 11.15 | 2 |
| 161 | 771294 | AA443624 | 0.01 | 0.20 | 27.04 | 8.33 | 1.36 | 71.43 | 2 |
| 162 | 194318 | H50655 | 0.53 | 14.38 | 26.99 | 0.69 | 27.83 | 52.45 | 2 |
| 163 | 769000 | AA425158 | 0.01 | 0.26 | 26.26 | 5.77 | 72.00 | 1.00 | 2 |
| 164 | 280699 | N47445 | 0.55 | 14.15 | 25.90 | 2.28 | 11.75 | 63.66 | 2 |
| 165 | 283379 | N52767 | 0.02 | 0.39 | 25.65 | 0.59 | 66.04 | 10.31 | 2 |
| 166 | 787856 | AA452139 | 0.01 | 0.29 | 25.59 | 4.23 | 62.71 | 9.84 | 2 |
| 167 | 27605 | R40018 | 0.07 | 1.90 | 25.57 | 2.05 | 68.60 | 6.05 | 2 |
| 168 | 430255 | AA010383 | 0.01 | 0.25 | 25.24 | 4.46 | 54.64 | 16.63 | 2 |
| 169 | 530958 | AA070437 | 0.01 | 0.25 | 25.21 | 2.29 | 67.88 | 5.46 | 2 |
| 170 | 53315 | R15813 | 0.01 | 0.25 | 25.10 | 2.87 | 64.40 | 8.05 | 2 |
| 171 | 687972 | AA236986 | 0.01 | 0.17 | 25.09 | 3.61 | 60.05 | 11.62 | 2 |
| 172 | 357884 | W94486 | 0.01 | 0.25 | 24.95 | 2.32 | 41.17 | 31.35 | 2 |
| 173 | 172517 | H19826 | 1.00 | 24.91 | 24.86 | 2.27 | 26.82 | 45.50 | 2 |
| 174 | 251806 | H96647 | 0.01 | 0.24 | 24.50 | 1.76 | 60.48 | 11.25 | 2 |
| 175 | 80643 | T57803 | 1.09 | 26.54 | 24.25 | 0.44 | 9.54 | 62.76 | 2 |
| 176 | 824025 | AA490945 | 0.31 | 7.41 | 24.24 | 0.95 | 28.65 | 43.13 | 2 |
| 177 | 29585 | R42112 | 0.04 | 0.84 | 23.75 | 8.09 | 59.71 | 3.46 | 2 |
| 178 | 825606 | AA504625 | 0.15 | 3.51 | 23.68 | 0.87 | 21.91 | 48.25 | 2 |
| 179 | 81050 | T70198 | 0.01 | 0.23 | 23.00 | 1.00 | 42.67 | 25.34 | 2 |
| 180 | 855910 | AA630328 | 0.01 | 0.16 | 22.37 | 4.98 | 30.37 | 31.76 | 2 |
| 181 | 34204 | R44936 | 0.17 | 3.74 | 22.30 | 3.82 | 51.95 | 11.14 | 2 |
| 182 | 591055 | AA161097 | 0.02 | 0.34 | 21.79 | 2.34 | 9.53 | 53.51 | 2 |
| 183 | 824270 | AA491261 | 0.17 | 3.65 | 21.71 | 0.88 | 10.05 | 54.20 | 2 |
| 184 | 46843 | H10072 | 0.01 | 0.14 | 21.70 | 39.83 | 1.50 | 23.77 | 2 |
| 185 | 79000 | T61938 | 1.90 | 40.77 | 21.44 | 37.52 | 23.72 | 3.08 | 2 |
| 186 | 259884 | N32904 | 0.91 | 19.16 | 20.97 | 4.40 | 10.88 | 47.62 | 2 |
| 187 | 686552 | AA255954 | 0.30 | 6.19 | 20.39 | 0.93 | 42.76 | 17.48 | 2 |
| 188 | 199337 | R95684 | 0.20 | 3.94 | 20.04 | 1.29 | 30.54 | 28.27 | 2 |
| 189 | 795831 | AA461508 | 0.01 | 0.20 | 19.97 | 1.00 | 38.72 | 20.17 | 2 |
| 190 | 826256 | AA520979 | 0.17 | 3.26 | 19.61 | 1.10 | 7.58 | 50.17 | 2 |
| 191 | 197265 | R86970 | 0.01 | 0.20 | 19.50 | 2.79 | 10.31 | 45.41 | 2 |
| 192 | 162310 | H28091 | 0.17 | 3.30 | 19.47 | 2.35 | 30.81 | 25.26 | 2 |
| 193 | 759163 | AA496022 | 0.01 | 0.19 | 19.37 | 7.38 | 1.00 | 49.74 | 2 |
| 194 | 809583 | AA456628 | 0.01 | 0.19 | 19.26 | 9.69 | 1.93 | 46.16 | 2 |
| 195 | 80338 | T65736 | 1.11 | 21.01 | 18.93 | 41.41 | 14.31 | 1.08 | 2 |
| 196 | 39722 | R54492 | 0.01 | 0.17 | 18.85 | 3.39 | 10.12 | 43.06 | 2 |
| 197 | 1049284 | AA620747 | 0.04 | 0.74 | 18.21 | 2.02 | 47.06 | 5.54 | 2 |
| 198 | 489076 | AA057195 | 0.02 | 0.35 | 18.01 | 4.49 | 38.83 | 10.71 | 2 |
| 199 | 839048 | AA487505 | 0.19 | 3.45 | 17.78 | 1.21 | 45.91 | 6.22 | 2 |
| 200 | 42907 | R60020 | 0.03 | 0.54 | 17.62 | 1.87 | 45.44 | 5.54 | 2 |
| 201 | 361899 | AA001376 | 0.01 | 0.17 | 17.44 | 1.97 | 34.15 | 16.22 | 2 |
| 202 | 823727 | AA489662 | 0.03 | 0.44 | 17.25 | 2.37 | 40.61 | 8.77 | 2 |
| 203 | 1031568 | AA609304 | 0.05 | 0.86 | 17.19 | 4.26 | 42.11 | 5.21 | 2 |
| 204 | 1410444 | AA857163 | 0.06 | 1.06 | 17.19 | 1.87 | 37.48 | 12.21 | 2 |
| 205 | 756629 | AA481481 | 0.01 | 0.11 | 16.78 | 14.65 | 1.51 | 34.18 | 2 |
| 206 | 266366 | N26559 | 0.02 | 0.29 | 16.62 | 2.86 | 39.78 | 7.22 | 2 |
| 207 | 283070 | N51297 | 0.07 | 1.14 | 16.47 | 2.84 | 40.90 | 5.65 | 2 |
| 208 | 753411 | AA410437 | 0.03 | 0.40 | 15.85 | 3.94 | 36.93 | 6.69 | 2 |
| 209 | 825726 | AA504838 | 0.19 | 2.94 | 15.84 | 2.63 | 16.52 | 28.36 | 2 |
| 210 | 746232 | AA417713 | 0.38 | 5.98 | 15.64 | 0.69 | 21.09 | 25.15 | 2 |
| 211 | 487766 | AA045175 | 0.02 | 0.30 | 15.57 | 4.69 | 22.78 | 19.22 | 2 |
| 212 | 811766 | AA463445 | 0.26 | 3.91 | 15.03 | 1.53 | 28.43 | 15.13 | 2 |
| 213 | 51599 | H18932 | 0.26 | 3.94 | 14.95 | 0.42 | 26.10 | 18.34 | 2 |
| 214 | 364865 | AA035745 | 0.04 | 0.54 | 14.60 | 36.64 | 0.27 | 6.87 | 2 |
| 215 | 785293 | AA476543 | 0.11 | 1.64 | 14.41 | 24.69 | 3.02 | 15.54 | 2 |
| 216 | 770860 | AA434388 | 0.01 | 0.14 | 14.40 | 1.00 | 18.93 | 23.25 | 2 |
| 217 | 1461609 | AA883800 | 0.25 | 3.51 | 14.24 | 10.07 | 4.19 | 28.45 | 2 |
| 218 | 856289 | AA774665 | 1.24 | 17.58 | 14.13 | 5.00 | 9.24 | 28.16 | 2 |
| 219 | 436402 | AA699601 | 0.16 | 2.19 | 14.09 | 0.88 | 36.28 | 5.10 | 2 |
| 220 | 418004 | W90705 | 0.30 | 4.17 | 13.89 | 2.99 | 11.04 | 27.66 | 2 |
| 221 | 41406 | R56149 | 0.01 | 0.13 | 13.76 | 17.85 | 18.97 | 4.48 | 2 |
| 222 | 26806 | R37738 | 0.10 | 1.31 | 13.68 | 2.42 | 26.44 | 12.18 | 2 |
| 223 | 771004 | AA427719 | 0.29 | 3.95 | 13.50 | 0.64 | 21.58 | 18.27 | 2 |
| 224 | 49499 | H15549 | 0.63 | 8.41 | 13.36 | 2.08 | 8.82 | 29.19 | 2 |
| 225 | 824074 | AA491227 | 0.02 | 0.26 | 13.22 | 3.39 | 18.08 | 18.18 | 2 |
| 226 | 1467166 | AA883181 | 0.08 | 1.05 | 13.14 | 3.08 | 29.16 | 7.18 | 2 |
| 227 | 284288 | N52193 | 0.01 | 0.08 | 13.00 | 10.97 | 1.54 | 26.50 | 2 |
| 228 | 364844 | AA035730 | 0.03 | 0.36 | 12.97 | 30.80 | 0.76 | 7.37 | 2 |
| 229 | 361122 | AA017379 | 0.01 | 0.13 | 12.88 | 0.65 | 12.28 | 25.70 | 2 |
| 230 | 1526789 | AA911236 | 0.65 | 8.32 | 12.81 | 1.86 | 8.17 | 28.39 | 2 |
| 231 | 33066 | R43915 | 0.06 | 0.71 | 12.79 | 7.14 | 29.01 | 2.23 | 2 |
| 232 | 1055121 | AA621355 | 0.02 | 0.25 | 12.67 | 6.31 | 28.51 | 3.21 | 2 |
| 233 | 530954 | AA070435 | 0.16 | 1.99 | 12.64 | 1.33 | 31.49 | 5.10 | 2 |
| 234 | 795446 | AA453616 | 0.01 | 0.13 | 12.56 | 22.57 | 14.55 | 0.58 | 2 |
| 235 | 757428 | AA437212 | 0.40 | 4.99 | 12.41 | 1.18 | 29.00 | 7.04 | 2 |
| 236 | 1049185 | AA620697 | 0.76 | 9.44 | 12.39 | 10.40 | 0.39 | 26.39 | 2 |
| 237 | 364352 | AA022496 | 0.01 | 0.12 | 12.38 | 1.96 | 14.19 | 20.98 | 2 |
| 238 | 47264 | H10713 | 0.30 | 3.72 | 12.36 | 2.11 | 23.87 | 11.09 | 2 |
| 239 | 71671 | T57927 | 0.01 | 0.12 | 12.24 | 6.29 | 29.43 | 1.00 | 2 |
| 240 | 1049079 | AA778717 | 0.03 | 0.31 | 12.10 | 3.28 | 15.47 | 17.55 | 2 |
| 241 | 785368 | AA476576 | 1.38 | 16.58 | 12.06 | 8.77 | 4.23 | 23.17 | 2 |
| 242 | 195845 | R92201 | 0.14 | 1.69 | 12.02 | 1.00 | 14.95 | 20.10 | 2 |
| 243 | 451649 | AA706901 | 1.40 | 16.82 | 11.98 | 4.34 | 18.70 | 12.88 | 2 |
| 244 | 739450 | AA477227 | 1.99 | 23.53 | 11.82 | 2.08 | 17.13 | 16.24 | 2 |
| 245 | 46461 | H09940 | 0.33 | 3.89 | 11.80 | 3.09 | 13.18 | 19.11 | 2 |
| 246 | 591157 | AA161188 | 0.02 | 0.18 | 11.71 | 0.58 | 20.54 | 14.01 | 2 |
| 247 | 33839 | R44816 | 0.96 | 11.24 | 11.67 | 3.60 | 19.31 | 12.09 | 2 |
| 248 | 357138 | W93523 | 0.02 | 0.20 | 11.63 | 10.87 | 20.61 | 3.40 | 2 |
| 249 | 366971 | AA026682 | 2.38 | 27.64 | 11.61 | 3.65 | 10.18 | 20.99 | 2 |
| 250 | 609228 | AA179161 | 0.01 | 0.11 | 11.46 | 4.58 | 5.70 | 24.11 | 2 |
| 251 | 592728 | AA160670 | 0.03 | 0.34 | 11.17 | 0.33 | 16.85 | 16.34 | 2 |
| 252 | 770854 | AA427737 | 0.09 | 0.95 | 11.06 | 2.56 | 22.78 | 7.83 | 2 |
| 253 | 193333 | H48070 | 0.36 | 3.90 | 10.90 | 0.92 | 17.96 | 13.80 | 2 |
| 254 | 795746 | AA460299 | 0.52 | 5.68 | 10.87 | 17.77 | 9.89 | 4.94 | 2 |
| 255 | 753428 | AA410434 | 0.57 | 6.12 | 10.84 | 0.51 | 15.37 | 16.62 | 2 |
| 256 | 298417 | N74131 | 0.03 | 0.31 | 10.83 | 2.09 | 20.21 | 10.20 | 2 |
| 257 | 795840 | AA461513 | 0.01 | 0.06 | 10.81 | 4.64 | 15.78 | 12.02 | 2 |
| 258 | 257955 | N30751 | 0.86 | 9.32 | 10.79 | 2.05 | 10.22 | 20.10 | 2 |
| 259 | 433155 | AA680136 | 0.01 | 0.11 | 10.76 | 22.91 | 1.00 | 8.36 | 2 |
| 260 | 767403 | AA417920 | 0.56 | 5.93 | 10.66 | 4.43 | 22.31 | 5.22 | 2 |
| 261 | 824802 | AA489073 | 0.77 | 8.09 | 10.50 | 0.74 | 8.99 | 21.77 | 2 |
| 262 | 814769 | AA454949 | 1.16 | 12.13 | 10.45 | 21.90 | 4.33 | 5.14 | 2 |
| 263 | 307138 | N93721 | 0.04 | 0.44 | 10.36 | 2.67 | 21.63 | 6.78 | 2 |
| 264 | 712401 | AA281784 | 0.08 | 0.87 | 10.32 | 0.88 | 14.48 | 15.60 | 2 |
| 265 | 203184 | H54451 | 0.41 | 4.21 | 10.31 | 0.86 | 6.31 | 23.76 | 2 |
| 266 | 277621 | N49389 | 0.54 | 5.55 | 10.21 | 0.77 | 14.23 | 15.64 | 2 |
| 267 | 729953 | AA412049 | 0.93 | 9.47 | 10.19 | 22.87 | 7.21 | 0.48 | 2 |
| 268 | 590640 | AA158035 | 0.01 | 0.12 | 10.11 | 11.51 | 17.15 | 1.65 | 2 |
| 269 | 197651 | R945O4 | 0.25 | 2.53 | 10.07 | 0.93 | 11.85 | 17.42 | 2 |
| 270 | 740457 | AA478036 | 1.60 | 16.05 | 10.05 | 2.67 | 6.06 | 21.42 | 2 |
| 271 | 898073 | AA598787 | 0.04 | 0.37 | 10.00 | 3.74 | 20.48 | 5.78 | 2 |
| 272 | 745248 | AA626167 | 0.24 | 2.42 | 9.97 | 8.61 | 20.18 | 1.12 | 2 |
| 273 | 855391 | AA664007 | 0.23 | 2.29 | 9.95 | 1.40 | 12.15 | 16.30 | 2 |
| 274 | 745490 | AA625979 | 0.11 | 1.05 | 9.95 | 12.71 | 3.07 | 14.06 | 2 |
| 275 | 725877 | AA292226 | 0.50 | 4.94 | 9.89 | 0.67 | 13.69 | 15.31 | 2 |
| 276 | 266135 | N21633 | 0.39 | 3.82 | 9.87 | 1.10 | 17.62 | 10.88 | 2 |
| 277 | 154720 | R55220 | 0.01 | 0.09 | 9.86 | 8.07 | 19.71 | 1.81 | 2 |
| 278 | 812994 | AA464615 | 0.02 | 0.21 | 9.80 | 5.59 | 23.34 | 0.46 | 2 |
| 279 | 391987 | AI003636 | 0.06 | 0.57 | 9.79 | 3.40 | 9.94 | 16.03 | 2 |
| 280 | 490387 | AA120779 | 0.02 | 0.21 | 9.77 | 1.35 | 22.17 | 5.79 | 2 |
| 281 | 431242 | AA682533 | 1.26 | 12.26 | 9.74 | 3.45 | 18.90 | 6.87 | 2 |
| 282 | 951305 | AA620528 | 0.62 | 5.97 | 9.68 | 1.31 | 9.16 | 18.56 | 2 |
| 283 | 590500 | AA157261 | 0.02 | 0.17 | 9.64 | 4.99 | 9.81 | 14.13 | 2 |
| 284 | 512116 | AA133590 | 0.64 | 6.12 | 9.62 | 0.66 | 21.91 | 6.28 | 2 |
| 285 | 590727 | AA156342 | 0.01 | 0.10 | 9.62 | 3.83 | 6.58 | 18.44 | 2 |
| 286 | 195695 | R89490 | 0.02 | 0.16 | 9.56 | 1.61 | 17.50 | 9.58 | 2 |
| 287 | 35804 | R46000 | 0.08 | 0.77 | 9.53 | 2.05 | 15.63 | 10.89 | 2 |
| 288 | 630013 | AA219060 | 0.15 | 1.40 | 9.48 | 1.15 | 11.85 | 15.44 | 2 |
| 289 | 320495 | W16659 | 0.01 | 0.09 | 9.47 | 10.45 | 1.00 | 16.97 | 2 |
| 290 | 430973 | AA678348 | 0.76 | 7.14 | 9.41 | 3.48 | 5.97 | 18.79 | 2 |
| 291 | 645565 | AA204830 | 0.62 | 5.85 | 9.41 | 4.56 | 7.97 | 15.70 | 2 |
| 292 | 866702 | AA679180 | 0.01 | 0.09 | 9.40 | 8.70 | 1.04 | 18.46 | 2 |
| 293 | 490959 | AA136664 | 0.53 | 5.02 | 9.39 | 1.84 | 5.32 | 21.00 | 2 |
| 294 | 435330 | AA699926 | 0.01 | 0.08 | 9.36 | 8.59 | 1.12 | 18.37 | 2 |
| 295 | 700792 | AA284072 | 0.01 | 0.06 | 9.34 | 11.43 | 1.54 | 15.06 | 2 |
| 296 | 346552 | W74377 | 0.02 | 0.19 | 9.32 | 3.79 | 15.51 | 8.67 | 2 |
| 297 | 281162 | N50962 | 0.02 | 0.22 | 9.31 | 0.97 | 12.53 | 14.44 | 2 |
| 298 | 814791 | AA455242 | 0.68 | 6.35 | 9.29 | 1.35 | 16.72 | 9.79 | 2 |
| 299 | 1627705 | AI017607 | 0.44 | 4.10 | 9.26 | 2.30 | 7.70 | 17.77 | 2 |
| 300 | 461488 | AA705047 | 0.10 | 0.97 | 9.22 | 12.49 | 10.86 | 4.33 | 2 |
| 301 | 230882 | R95962 | 0.10 | 0.87 | 9.18 | 2.37 | 19.73 | 5.45 | 2 |
| 302 | 781047 | AA446462 | 1.08 | 9.89 | 9.17 | 4.58 | 11.63 | 11.30 | 2 |
| 303 | 46694 | H10192 | 0.35 | 3.21 | 9.15 | 8.17 | 14.83 | 4.46 | 2 |
| 304 | 199577 | R96579 | 0.27 | 2.44 | 9.12 | 0.81 | 13.04 | 13.51 | 2 |
| 305 | 141726 | R69584 | 0.03 | 0.26 | 9.09 | 3.46 | 10.73 | 13.08 | 2 |
| 306 | 320865 | W44766 | 0.03 | 0.26 | 9.07 | 12.50 | 0.36 | 14.35 | 2 |
| 307 | 490556 | AA100696 | 0.01 | 0.09 | 9.07 | 5.08 | 1.00 | 21.12 | 2 |
| 308 | 266259 | N26515 | 0.35 | 3.21 | 9.06 | 1.65 | 10.87 | 14.67 | 2 |
| 309 | 627541 | AA192527 | 0.40 | 3.62 | 9.05 | 1.54 | 20.24 | 5.38 | 2 |
| 310 | 877632 | AA488175 | 1.17 | 10.62 | 9.04 | 2.44 | 15.22 | 9.46 | 2 |
| 311 | 594758 | AA172056 | 1.30 | 11.71 | 9.03 | 12.54 | 13.45 | 1.10 | 2 |
| 312 | 951241 | AA620485 | 1.98 | 17.81 | 8.98 | 10.05 | 0.30 | 16.60 | 2 |
| 313 | 730036 | AA416970 | 0.02 | 0.21 | 8.96 | 0.65 | 20.84 | 5.40 | 2 |
| 314 | 342640 | W68220 | 0.67 | 5.97 | 8.87 | 11.72 | 2.96 | 11.94 | 2 |
| 315 | 175533 | H41203 | 0.38 | 3.32 | 8.84 | 0.92 | 17.16 | 8.45 | 2 |
| 316 | 50227 | H16772 | 0.25 | 2.16 | 8.81 | 9.92 | 2.08 | 14.43 | 2 |
| 317 | 884511 | AA629999 | 4.51 | 39.21 | 8.70 | 0.36 | 17.44 | 8.30 | 2 |
| 318 | 1048995 | AA778646 | 0.09 | 0.80 | 8.69 | 1.34 | 6.64 | 18.10 | 2 |
| 319 | 242952 | H95638 | 5.65 | 48.92 | 8.66 | 3.86 | 6.65 | 15.48 | 2 |
| 320 | 878544 | AA775863 | 3.96 | 34.28 | 8.65 | 1.87 | 16.58 | 7.50 | 2 |
| 321 | 897546 | AA496998 | 0.01 | 0.09 | 8.56 | 10.33 | 1.00 | 14.35 | 2 |
| 322 | 462926 | AA682321 | 0.60 | 5.11 | 8.53 | 6.22 | 0.52 | 18.86 | 2 |
| 323 | 71977 | T52311 | 0.02 | 0.14 | 8.51 | 7.42 | 14.21 | 3.90 | 2 |
| 324 | 300615 | N80764 | 0.83 | 6.98 | 8.42 | 0.78 | 6.50 | 17.99 | 2 |
| 325 | 324717 | W47364 | 1.13 | 9.51 | 8.39 | 1.45 | 12.46 | 11.27 | 2 |
| 326 | 825416 | AA504265 | 0.40 | 3.36 | 8.39 | 1.30 | 11.51 | 12.36 | 2 |
| 327 | 484535 | AA036974 | 0.01 | 0.08 | 8.39 | 1.81 | 6.16 | 17.21 | 2 |
| 328 | 840150 | AA485265 | 0.02 | 0.19 | 8.35 | 4.95 | 13.73 | 6.37 | 2 |
| 329 | 809557 | AA455786 | 0.53 | 4.39 | 8.32 | 3.89 | 6.65 | 14.40 | 2 |
| 330 | 296429 | N74617 | 1.62 | 13.45 | 8.31 | 2.41 | 6.27 | 16.24 | 2 |
| 331 | 842968 | AA488324 | 1.30 | 10.75 | 8.30 | 5.22 | 0.41 | 19.27 | 2 |
| 332 | 37980 | R61372 | 0.13 | 1.09 | 8.29 | 8.74 | 13.18 | 2.96 | 2 |
| 333 | 280882 | N50806 | 0.02 | 0.15 | 8.25 | 9.98 | 1.67 | 13.09 | 2 |
| 334 | 380884 | AA058576 | 0.18 | 1.49 | 8.21 | 2.23 | 11.14 | 11.26 | 2 |
| 335 | 503581 | AA131239 | 0.02 | 0.14 | 8.15 | 5.83 | 0.56 | 18.07 | 2 |
| 336 | 358599 | W96216 | 0.03 | 0.25 | 8.13 | 3.04 | 13.64 | 7.71 | 2 |
| 337 | 30148 | R41329 | 2.63 | 21.34 | 8.13 | 4.35 | 5.10 | 14.93 | 2 |
| 338 | 203275 | H54752 | 1.29 | 10.44 | 8.09 | 5.06 | 3.98 | 15.23 | 2 |
| 339 | 433253 | AA699427 | 0.76 | 6.11 | 8.08 | 1.22 | 13.54 | 9.48 | 2 |
| 340 | 838776 | AA457576 | 0.03 | 0.22 | 8.03 | 0.38 | 18.34 | 5.36 | 2 |
| 341 | 1292182 | AA705825 | 0.03 | 0.28 | 8.02 | 3.42 | 7.53 | 13.11 | 2 |
| 342 | 230261 | H94944 | 0.01 | 0.08 | 8.02 | 2.25 | 8.34 | 13.46 | 2 |
| 343 | 193892 | H51765 | 1.10 | 8.81 | 8.00 | 0.92 | 14.68 | 8.40 | 2 |
| 344 | 701625 | AA284172 | 0.29 | 2.29 | 7.89 | 1.06 | 13.94 | 8.68 | 2 |
| 345 | 38833 | R50775 | 0.66 | 5.20 | 7.83 | 2.27 | 15.21 | 6.01 | 2 |
| 346 | 190325 | H29897 | 0.37 | 2.89 | 7.77 | 4.86 | 11.97 | 6.49 | 2 |
| 347 | 745393 | AA625764 | 0.08 | 0.60 | 7.77 | 3.94 | 9.24 | 10.12 | 2 |
| 348 | 782259 | AA431741 | 0.75 | 5.81 | 7.75 | 11.54 | 7.24 | 4.46 | 2 |
| 349 | 502444 | AA156795 | 0.02 | 0.14 | 7.73 | 0.57 | 7.43 | 15.20 | 2 |
| 350 | 1505919 | AA906257 | 0.05 | 0.35 | 7.72 | 4.23 | 8.93 | 10.00 | 2 |
| 351 | 121251 | T96718 | 3.19 | 24.59 | 7.70 | 7.55 | 4.17 | 11.39 | 2 |
| 352 | 73596 | T55607 | 1.74 | 13.42 | 7.70 | 4.60 | 7.66 | 10.84 | 2 |
| 353 | 204644 | H57082 | 0.27 | 2.05 | 7.70 | 1.18 | 12.63 | 9.28 | 2 |
| 354 | 322461 | W15305 | 0.22 | 1.68 | 7.70 | 6.23 | 3.58 | 13.28 | 2 |
| 355 | 810661 | AA463982 | 0.01 | 0.08 | 7.69 | 7.96 | 14.11 | 1.00 | 2 |
| 356 | 882510 | AA676460 | 34.04 | 261.61 | 7.68 | 3.35 | 8.00 | 11.71 | 2 |
| 357 | 46927 | H10231 | 0.07 | 0.53 | 7.67 | 2.09 | 11.89 | 9.03 | 2 |
| 358 | 46740 | H10068 | 0.03 | 0.25 | 7.66 | 1.41 | 14.72 | 6.84 | 2 |
| 359 | 1035664 | AA780365 | 0.13 | 1.03 | 7.65 | 12.36 | 8.59 | 2.00 | 2 |
| 360 | 345743 | W72666 | 2.36 | 18.06 | 7.64 | 1.23 | 16.02 | 5.66 | 2 |
| 361 | 120695 | T95804 | 0.01 | 0.08 | 7.61 | 0.83 | 12.70 | 9.32 | 2 |
| 362 | 725340 | AA291773 | 0.72 | 5.46 | 7.60 | 1.27 | 5.74 | 15.79 | 2 |
| 363 | 530820 | AA070331 | 0.01 | 0.04 | 7.55 | 10.53 | 4.96 | 7.15 | 2 |
| 364 | 448032 | AA702802 | 0.01 | 0.08 | 7.54 | 5.64 | 1.00 | 15.99 | 2 |
| 365 | 39833 | R53456 | 0.97 | 7.28 | 7.53 | 0.35 | 12.36 | 9.88 | 2 |
| 366 | 770879 | AA434403 | 0.75 | 5.64 | 7.53 | 3.47 | 10.88 | 8.23 | 2 |
| 367 | 868169 | AA633835 | 0.16 | 1.18 | 7.49 | 1.25 | 5.39 | 15.83 | 2 |
| 368 | 811166 | AA485749 | 0.01 | 0.08 | 7.46 | 12.26 | 0.92 | 9.18 | 2 |
| 369 | 83666 | T61122 | 0.03 | 0.20 | 7.44 | 12.64 | 4.41 | 5.28 | 2 |
| 370 | 128243 | R12473 | 0.83 | 6.14 | 7.44 | 1.30 | 8.97 | 12.04 | 2 |
| 371 | 37538 | R49644 | 0.04 | 0.31 | 7.41 | 0.99 | 11.96 | 9.29 | 2 |
| 372 | 435076 | AA701455 | 2.95 | 21.88 | 7.41 | 10.35 | 6.94 | 4.93 | 2 |
| 373 | 309288 | N93924 | 3.23 | 23.90 | 7.39 | 5.03 | 4.68 | 12.46 | 2 |
| 374 | 843163 | AA488367 | 7.80 | 57.27 | 7.34 | 3.00 | 6.84 | 12.18 | 2 |
| 375 | 278729 | N62936 | 1.35 | 9.91 | 7.34 | 0.34 | 13.66 | 8.02 | 2 |
| 376 | 66317 | T66816 | 3.88 | 28.27 | 7.29 | 6.83 | 0.63 | 14.40 | 2 |
| 377 | 451907 | AA706968 | 2.08 | 15.11 | 7.27 | 11.07 | 0.08 | 10.67 | 2 |
| 378 | 266631 | N22766 | 0.05 | 0.35 | 7.24 | 1.57 | 9.92 | 10.22 | 2 |
| 379 | 32756 | R43308 | 0.30 | 2.20 | 7.23 | 7.23 | 2.06 | 12.41 | 2 |
| 380 | 50904 | H19234 | 0.16 | 1.17 | 7.23 | 1.81 | 13.06 | 6.82 | 2 |
| 381 | 565734 | AA135809 | 1.24 | 8.99 | 7.23 | 2.92 | 12.65 | 6.11 | 2 |
| 382 | 85670 | T62072 | 0.80 | 5.80 | 7.23 | 4.11 | 7.03 | 10.54 | 2 |
| 383 | 359722 | AA011182 | 0.04 | 0.31 | 7.22 | 1.78 | 13.46 | 6.43 | 2 |
| 384 | 251936 | H97488 | 4.68 | 33.83 | 7.22 | 4.39 | 11.90 | 5.38 | 2 |
| 385 | 796114 | AA460952 | 0.94 | 6.75 | 7.20 | 3.01 | 9.60 | 8.99 | 2 |
| 386 | 809828 | AA455521 | 0.64 | 4.64 | 7.20 | 2.72 | 7.61 | 11.27 | 2 |
| 387 | 825282 | AA504201 | 2.23 | 15.96 | 7.17 | 2.25 | 8.89 | 10.38 | 2 |
| 388 | 809567 | AA456611 | 0.01 | 0.10 | 7.15 | 4.40 | 6.35 | 10.69 | 2 |
| 389 | 897271 | AA677655 | 0.02 | 0.11 | 7.14 | 0.87 | 8.75 | 11.80 | 2 |
| 390 | 1416782 | AA894557 | 14.77 | 105.24 | 7.13 | 13.51 | 0.03 | 7.84 | 2 |
| 391 | 814779 | AA455237 | 2.03 | 14.44 | 7.12 | 2.47 | 11.28 | 7.62 | 2 |
| 392 | 785845 | AA449118 | 0.09 | 0.62 | 7.11 | 13.68 | 2.34 | 5.32 | 2 |
| 393 | 199644 | R96520 | 0.21 | 1.51 | 7.11 | 1.14 | 11.35 | 8.84 | 2 |
| 394 | 278531 | N66158 | 13.50 | 95.95 | 7.11 | 2.00 | 12.50 | 6.81 | 2 |
| 395 | 283173 | N45236 | 2.09 | 14.78 | 7.08 | 11.72 | 5.87 | 3.65 | 2 |
| 396 | 143365 | R74203 | 0.01 | 0.08 | 7.03 | 4.65 | 9.95 | 6.50 | 2 |
| 397 | 247050 | N53940 | 0.29 | 2.01 | 7.02 | 1.68 | 7.50 | 11.87 | 2 |
| 398 | 1637343 | AI015359 | 14.33 | 100.32 | 7.00 | 4.74 | 7.07 | 9.20 | 2 |
| 399 | 345330 | W72556 | 0.04 | 0.28 | 7.00 | 0.85 | 5.67 | 14.49 | 2 |
| 400 | 415906 | W86196 | 0.06 | 0.44 | 6.96 | 3.01 | 6.52 | 11.35 | 2 |
| 401 | 82215 | T68878 | 0.10 | 0.68 | 6.95 | 2.37 | 9.22 | 9.27 | 2 |
| 402 | 811067 | AA485453 | 5.22 | 36.26 | 6.95 | 2.09 | 8.38 | 10.38 | 2 |
| 403 | 79254 | T58146 | 0.01 | 0.07 | 6.93 | 14.50 | 5.14 | 1.16 | 2 |
| 404 | 40773 | R56046 | 0.06 | 0.39 | 6.93 | 6.40 | 11.26 | 3.14 | 2 |
| 405 | 264502 | N20305 | 0.53 | 3.69 | 6.92 | 6.49 | 4.41 | 9.86 | 2 |
| 406 | 814225 | AA465238 | 1.08 | 7.46 | 6.92 | 4.76 | 10.31 | 5.69 | 2 |
| 407 | 1500162 | AA886739 | 0.55 | 3.78 | 6.90 | 2.34 | 9.72 | 8.65 | 2 |
| 408 | 856585 | AA669222 | 0.01 | 0.06 | 6.89 | 7.04 | 1.10 | 12.53 | 2 |
| 409 | 787938 | AA452278 | 0.40 | 2.74 | 6.88 | 10.95 | 1.51 | 8.19 | 2 |
| 410 | 586650 | AA129135 | 0.69 | 4.75 | 6.85 | 2.73 | 8.48 | 9.34 | 2 |
| 411 | 1641367 | AI025259 | 0.12 | 0.83 | 6.84 | 1.77 | 7.15 | 11.61 | 2 |
| 412 | 726791 | AA398406 | 0.04 | 0.25 | 6.83 | 1.52 | 13.88 | 5.09 | 2 |
| 413 | 131316 | R23055 | 1.19 | 8.14 | 6.82 | 3.20 | 5.03 | 12.23 | 2 |
| 414 | 204580 | H56903 | 0.41 | 2.79 | 6.80 | 1.22 | 12.71 | 6.48 | 2 |
| 415 | 681906 | AA256231 | 0.01 | 0.08 | 6.79 | 4.75 | 10.24 | 5.38 | 2 |
| 416 | 214604 | H71230 | 0.05 | 0.34 | 6.79 | 9.29 | 4.78 | 6.30 | 2 |
| 417 | 46091 | H08595 | 1.32 | 8.93 | 6.79 | 6.37 | 9.34 | 4.64 | 2 |
| 418 | 359119 | AA010065 | 4.09 | 27.70 | 6.77 | 5.57 | 3.78 | 10.97 | 2 |
| 419 | 809503 | AA454562 | 0.24 | 1.65 | 6.77 | 12.08 | 7.25 | 0.98 | 2 |
| 420 | 811582 | AA454597 | 1.50 | 10.08 | 6.74 | 9.56 | 1.82 | 8.83 | 2 |
| 421 | 51083 | H17139 | 0.04 | 0.30 | 6.72 | 7.84 | 4.68 | 7.65 | 2 |
| 422 | 1031885 | AA609723 | 0.54 | 3.61 | 6.72 | 8.02 | 8.01 | 4.14 | 2 |
| 423 | 725677 | AA398949 | 1.11 | 7.44 | 6.71 | 2.08 | 10.73 | 7.33 | 2 |
| 424 | 814246 | AA465593 | 9.85 | 65.62 | 6.66 | 3.37 | 9.66 | 6.95 | 2 |
| 425 | 950445 | AA599092 | 2.82 | 18.79 | 6.65 | 7.13 | 4.39 | 8.44 | 2 |
| 426 | 813149 | AA456695 | 0.70 | 4.67 | 6.65 | 7.85 | 4.47 | 7.62 | 2 |
| 427 | 768172 | AA424790 | 1.17 | 7.75 | 6.64 | 11.27 | 0.58 | 8.09 | 2 |
| 428 | 204214 | H59203 | 0.68 | 4.52 | 6.64 | 8.09 | 3.71 | 8.12 | 2 |
| 429 | 781362 | AA448400 | 0.01 | 0.07 | 6.62 | 7.97 | 10.05 | 1.85 | 2 |
| 430 | 73638 | T55728 | 9.61 | 63.60 | 6.62 | 1.16 | 9.16 | 9.54 | 2 |
| 431 | 1468597 | AA884622 | 0.01 | 0.07 | 6.59 | 7.52 | 7.44 | 4.80 | 2 |
| 432 | 375827 | AA039851 | 0.02 | 0.12 | 6.57 | 2.44 | 7.59 | 9.68 | 2 |
| 433 | 43662 | H05645 | 0.06 | 0.42 | 6.54 | 10.89 | 5.69 | 3.05 | 2 |
| 434 | 51460 | H20847 | 1.12 | 7.31 | 6.53 | 5.27 | 9.69 | 4.63 | 2 |
| 435 | 281733 | N48075 | 0.41 | 2.64 | 6.52 | 4.93 | 8.97 | 5.67 | 2 |
| 436 | 72441 | T51617 | 0.04 | 0.24 | 6.51 | 1.47 | 11.33 | 6.74 | 2 |
| 437 | 32134 | R43328 | 1.01 | 6.55 | 6.50 | 2.35 | 11.59 | 5.55 | 2 |
| 438 | 1573251 | AA953357 | 1.34 | 8.71 | 6.49 | 10.79 | 2.65 | 6.04 | 2 |
| 439 | 160233 | H21943 | 1.30 | 8.44 | 6.48 | 4.26 | 5.16 | 10.01 | 2 |
| 440 | 773564 | AA428186 | 0.02 | 0.12 | 6.46 | 2.50 | 9.29 | 7.59 | 2 |
| 441 | 487297 | AA045508 | 1.13 | 7.26 | 6.45 | 3.07 | 8.32 | 7.97 | 2 |
| 442 | 877784 | AA626788 | 4.30 | 27.63 | 6.43 | 1.38 | 9.13 | 8.78 | 2 |
| 443 | 430007 | AA034115 | 0.26 | 1.70 | 6.43 | 4.03 | 5.78 | 9.48 | 2 |
| 444 | 788488 | AA452542 | 0.07 | 0.45 | 6.42 | 2.05 | 11.14 | 6.08 | 2 |
| 445 | 786078 | AA448664 | 1.05 | 6.74 | 6.41 | 2.85 | 6.70 | 9.70 | 2 |
| 446 | 395955 | AA757564 | 0.13 | 0.82 | 6.41 | 5.00 | 4.97 | 9.25 | 2 |
| 447 | 159462 | H15910 | 0.03 | 0.21 | 6.41 | 5.69 | 12.25 | 1.28 | 2 |
| 448 | 194717 | R89846 | 0.26 | 1.66 | 6.39 | 0.90 | 6.06 | 12.22 | 2 |
| 449 | 782476 | AA431753 | 0.76 | 4.82 | 6.38 | 8.52 | 6.40 | 4.22 | 2 |
| 450 | 277112 | N39611 | 0.59 | 3.76 | 6.37 | 2.87 | 7.88 | 8.37 | 2 |
| 451 | 772410 | AA405533 | 0.71 | 4.54 | 6.37 | 1.54 | 5.53 | 12.04 | 2 |
| 452 | 196824 | R93068 | 0.24 | 1.50 | 6.36 | 0.76 | 12.20 | 6.13 | 2 |
| 453 | 814211 | AA465223 | 1.01 | 6.46 | 6.36 | 1.48 | 6.38 | 11.23 | 2 |
| 454 | 814444 | AA459244 | 0.77 | 4.87 | 6.31 | 1.06 | 8.03 | 9.86 | 2 |
| 455 | 486538 | AA042812 | 8.25 | 51.96 | 6.30 | 1.79 | 10.78 | 6.32 | 2 |
| 456 | 292936 | N63744 | 0.72 | 4.53 | 6.29 | 3.26 | 6.93 | 8.69 | 2 |
| 457 | 416075 | W85878 | 0.93 | 5.87 | 6.29 | 2.64 | 8.05 | 8.17 | 2 |
| 458 | 289496 | N63988 | 0.35 | 2.21 | 6.28 | 1.45 | 10.13 | 7.26 | 2 |
| 459 | 302221 | N77828 | 0.22 | 1.37 | 6.27 | 7.40 | 2.46 | 8.95 | 2 |
| 460 | 203179 | H54659 | 0.25 | 1.58 | 6.27 | 2.11 | 8.82 | 7.88 | 2 |
| 461 | 34254 | R44201 | 0.02 | 0.15 | 6.27 | 2.51 | 6.21 | 10.08 | 2 |
| 462 | 267135 | N24869 | 0.37 | 2.29 | 6.25 | 0.27 | 5.01 | 13.49 | 2 |
| 463 | 769857 | AA430367 | 2.19 | 13.69 | 6.25 | 13.18 | 0.27 | 5.30 | 2 |
| 464 | 130004 | R11605 | 0.38 | 2.35 | 6.25 | 3.19 | 5.05 | 10.52 | 2 |
| 465 | 435957 | AA701978 | 0.66 | 4.11 | 6.23 | 0.87 | 12.34 | 5.48 | 2 |
| 466 | 23073 | R38539 | 0.22 | 1.36 | 6.22 | 0.68 | 8.92 | 9.06 | 2 |
| 467 | 52128 | H22568 | 1.94 | 12.01 | 6.20 | 1.35 | 7.60 | 9.66 | 2 |
| 468 | 320602 | W31389 | 0.01 | 0.08 | 6.20 | 11.05 | 0.76 | 6.77 | 2 |
| 469 | 813586 | AA447662 | 1.97 | 12.17 | 6.19 | 4.19 | 5.59 | 8.79 | 2 |
| 470 | 277476 | N56875 | 0.05 | 0.33 | 6.16 | 1.20 | 10.69 | 6.60 | 2 |
| 471 | 1470195 | AA865924 | 0.42 | 2.57 | 6.15 | 8.48 | 2.18 | 7.78 | 2 |
| 472 | 854701 | AA630104 | 2.60 | 15.94 | 6.13 | 0.57 | 7.55 | 10.29 | 2 |
| 473 | 770992 | AA430744 | 2.03 | 12.43 | 6.13 | 6.15 | 2.69 | 9.56 | 2 |
| 474 | 1032080 | AA609891 | 0.47 | 2.89 | 6.13 | 1.59 | 7.76 | 9.04 | 2 |
| 475 | 50983 | H18017 | 0.02 | 0.14 | 6.12 | 7.62 | 5.90 | 4.83 | 2 |
| 476 | 195817 | R92186 | 0.39 | 2.40 | 6.11 | 1.12 | 9.73 | 7.48 | 2 |
| 477 | 781109 | AA430052 | 2.62 | 16.02 | 6.11 | 2.39 | 8.86 | 7.07 | 2 |
| 478 | 814303 | AA459106 | 4.36 | 26.54 | 6.09 | 1.52 | 11.73 | 5.03 | 2 |
| 479 | 256680 | H96392 | 0.81 | 4.93 | 6.06 | 3.55 | 6.77 | 7.86 | 2 |
| 480 | 489805 | AA102068 | 0.01 | 0.08 | 6.06 | 6.43 | 4.55 | 7.19 | 2 |
| 481 | 773147 | AA425404 | 1.31 | 7.89 | 6.04 | 4.66 | 8.09 | 5.37 | 2 |
| 482 | 951068 | AA620437 | 0.16 | 0.94 | 6.02 | 9.62 | 3.03 | 5.40 | 2 |
| 483 | 813410 | AA458646 | 1.29 | 7.71 | 6.00 | 2.89 | 9.92 | 5.20 | 2 |
| 484 | 814080 | AA465353 | 1.48 | 8.89 | 5.99 | 0.97 | 9.75 | 7.25 | 2 |
| 485 | 825284 | AA504202 | 1.39 | 8.34 | 5.98 | 2.44 | 7.62 | 7.87 | 2 |
| 486 | 276972 | N39233 | 0.50 | 2.99 | 5.98 | 1.57 | 8.57 | 7.79 | 2 |
| 487 | 1505686 | AA879423 | 0.09 | 0.55 | 5.96 | 2.30 | 9.04 | 6.52 | 2 |
| 488 | 563860 | AA101173 | 0.12 | 0.69 | 5.93 | 0.97 | 11.46 | 5.38 | 2 |
| 489 | 289645 | N62866 | 0.31 | 1.85 | 5.93 | 11.41 | 0.30 | 6.09 | 2 |
| 490 | 272888 | N36008 | 2.68 | 15.84 | 5.91 | 1.63 | 7.34 | 8.76 | 2 |
| 491 | 271076 | N29918 | 0.54 | 3.18 | 5.90 | 7.03 | 3.15 | 7.53 | 2 |
| 492 | 284783 | N59866 | 0.06 | 0.36 | 5.90 | 6.66 | 8.19 | 2.86 | 2 |
| 493 | 1048949 | AA778603 | 0.13 | 0.76 | 5.88 | 0.84 | 8.58 | 8.22 | 2 |
| 494 | 246549 | N73252 | 0.43 | 2.56 | 5.88 | 1.87 | 6.81 | 8.94 | 2 |
| 495 | 357531 | W94009 | 1.37 | 8.00 | 5.86 | 4.55 | 5.43 | 7.60 | 2 |
| 496 | 1343468 | AA709271 | 0.69 | 4.03 | 5.83 | 8.54 | 2.32 | 6.64 | 2 |
| 497 | 150623 | H02158 | 0.01 | 0.06 | 5.82 | 6.78 | 1.00 | 9.69 | 2 |
| 498 | 809535 | AA454585 | 7.47 | 43.39 | 5.81 | 3.50 | 5.22 | 8.71 | 2 |
| 499 | 287637 | N59136 | 1.87 | 10.85 | 5.80 | 4.53 | 5.30 | 7.58 | 2 |
| 500 | 745360 | AA625662 | 5.55 | 32.21 | 5.80 | 3.92 | 7.27 | 6.22 | 2 |
| 501 | 814995 | AA465090 | 0.83 | 4.83 | 5.80 | 2.24 | 9.55 | 5.62 | 2 |
| 502 | 1031113 | AA609914 | 0.04 | 0.22 | 5.79 | 1.21 | 10.89 | 5.26 | 2 |
| 503 | 74738 | T57359 | 2.36 | 13.63 | 5.78 | 3.39 | 5.86 | 8.09 | 2 |
| 504 | 147385 | H01332 | 0.03 | 0.16 | 5.76 | 4.97 | 6.03 | 6.29 | 2 |
| 505 | 506483 | AA708605 | 0.10 | 0.59 | 5.76 | 1.96 | 10.22 | 5.11 | 2 |
| 506 | 287581 | N62128 | 0.82 | 4.73 | 5.76 | 5.27 | 10.45 | 1.55 | 2 |
| 507 | 28475 | R13434 | 0.46 | 2.62 | 5.74 | 1.10 | 7.72 | 8.40 | 2 |
| 508 | 38510 | R50752 | 0.59 | 3.37 | 5.74 | 2.46 | 7.30 | 7.45 | 2 |
| 509 | 46862 | H10788 | 1.07 | 6.12 | 5.73 | 2.42 | 9.02 | 5.77 | 2 |
| 510 | 191546 | H37817 | 0.24 | 1.35 | 5.72 | 0.89 | 6.27 | 10.01 | 2 |
| 511 | 27817 | R40481 | 0.03 | 0.15 | 5.71 | 2.14 | 9.62 | 5.36 | 2 |
| 512 | 504826 | AA150777 | 0.83 | 4.73 | 5.69 | 6.56 | 5.66 | 4.86 | 2 |
| 513 | 34063 | R45952 | 0.28 | 1.58 | 5.68 | 5.72 | 9.78 | 1.55 | 2 |
| 514 | 291374 | N72274 | 1.00 | 5.71 | 5.68 | 1.00 | 6.51 | 9.54 | 2 |
| 515 | 282104 | N51498 | 0.51 | 2.92 | 5.68 | 5.83 | 9.35 | 1.87 | 2 |
| 516 | 196037 | R89363 | 0.41 | 2.35 | 5.68 | 1.17 | 8.12 | 7.73 | 2 |
| 517 | 667598 | AA228130 | 0.90 | 5.08 | 5.66 | 3.60 | 5.70 | 7.68 | 2 |
| 518 | 433474 | AA699560 | 3.28 | 18.53 | 5.65 | 8.40 | 2.36 | 6.19 | 2 |
| 519 | 856592 | AA669218 | 1.19 | 6.68 | 5.64 | 10.55 | 1.37 | 5.00 | 2 |
| 520 | 395609 | AA757588 | 0.05 | 0.25 | 5.62 | 0.55 | 7.77 | 8.55 | 2 |
| 521 | 770979 | AA427400 | 0.02 | 0.10 | 5.62 | 5.02 | 0.44 | 11.39 | 2 |
| 522 | 357344 | W93500 | 0.01 | 0.06 | 5.62 | 5.85 | 8.42 | 2.59 | 2 |
| 523 | 146882 | R80790 | 4.49 | 25.20 | 5.62 | 6.88 | 4.48 | 5.48 | 2 |
| 524 | 220700 | H93424 | 0.96 | 5.38 | 5.61 | 5.49 | 4.88 | 6.45 | 2 |
| 525 | 950926 | AA608730 | 3.17 | 17.75 | 5.60 | 8.06 | 1.26 | 7.48 | 2 |
| 526 | 361688 | W96197 | 0.01 | 0.07 | 5.60 | 0.80 | 10.00 | 5.99 | 2 |
| 527 | 193139 | H47315 | 0.03 | 0.17 | 5.59 | 1.43 | 9.88 | 5.46 | 2 |
| 528 | 121798 | T98201 | 0.72 | 4.03 | 5.59 | 6.89 | 7.41 | 2.47 | 2 |
| 529 | 471707 | AA035580 | 0.66 | 3.65 | 5.55 | 6.12 | 2.85 | 7.67 | 2 |
| 530 | 51465 | H23983 | 0.07 | 0.40 | 5.54 | 4.68 | 6.93 | 5.02 | 2 |
| 531 | 878798 | AA670408 | 23.98 | 132.57 | 5.53 | 1.02 | 7.04 | 8.52 | 2 |
| 532 | 1505898 | AA907714 | 0.07 | 0.41 | 5.52 | 2.10 | 8.01 | 6.45 | 2 |
| 533 | 839081 | AA487608 | 12.19 | 66.94 | 5.49 | 7.57 | 0.09 | 8.81 | 2 |
| 534 | 491615 | AA115559 | 0.93 | 5.08 | 5.49 | 3.62 | 6.29 | 6.56 | 2 |
| 535 | 130845 | R22308 | 2.02 | 11.11 | 5.49 | 0.87 | 7.74 | 7.85 | 2 |
| 536 | 241432 | H80685 | 1.63 | 8.93 | 5.48 | 7.11 | 7.64 | 1.70 | 2 |
| 537 | 197206 | R92812 | 0.98 | 5.38 | 5.47 | 0.67 | 8.14 | 7.61 | 2 |
| 538 | 773170 | AA428514 | 0.05 | 0.26 | 5.45 | 1.75 | 8.29 | 6.30 | 2 |
| 539 | 256720 | H96356 | 0.01 | 0.05 | 5.44 | 1.86 | 6.97 | 7.49 | 2 |
| 540 | 361069 | AA017213 | 0.06 | 0.31 | 5.44 | 3.64 | 5.09 | 7.59 | 2 |
| 541 | 195786 | R89287 | 0.19 | 1.05 | 5.44 | 1.09 | 6.79 | 8.44 | 2 |
| 542 | 1466998 | AA884428 | 0.08 | 0.45 | 5.43 | 2.62 | 8.15 | 5.51 | 2 |
| 543 | 451805 | AA706829 | 6.18 | 33.51 | 5.42 | 1.61 | 5.96 | 8.69 | 2 |
| 544 | 898097 | AA598803 | 1.04 | 5.65 | 5.42 | 6.63 | 5.49 | 4.14 | 2 |
| 545 | 1467751 | AA883100 | 0.11 | 0.59 | 5.41 | 1.84 | 6.69 | 7.71 | 2 |
| 546 | 814350 | AA458827 | 1.32 | 7.13 | 5.38 | 2.06 | 5.32 | 8.77 | 2 |
| 547 | 459947 | AA779383 | 0.30 | 1.59 | 5.37 | 4.66 | 5.58 | 5.87 | 2 |
| 548 | 789204 | AA450205 | 2.16 | 11.60 | 5.37 | 5.05 | 3.79 | 7.27 | 2 |
| 549 | 431245 | AA682545 | 1.90 | 10.19 | 5.37 | 3.91 | 5.22 | 6.98 | 2 |
| 550 | 741790 | AA402965 | 2.17 | 11.61 | 5.35 | 1.13 | 6.68 | 8.24 | 2 |
| 551 | 460114 | AA676840 | 0.10 | 0.55 | 5.34 | 3.39 | 7.08 | 5.55 | 2 |
| 552 | 357285 | W93682 | 0.18 | 0.94 | 5.33 | 3.66 | 6.07 | 6.26 | 2 |
| 553 | 951125 | AA620556 | 6.73 | 35.80 | 5.32 | 4.34 | 5.23 | 6.39 | 2 |
| 554 | 156473 | R73525 | 0.01 | 0.05 | 5.32 | 8.91 | 1.01 | 6.03 | 2 |
| 555 | 129664 | R16676 | 1.18 | 6.28 | 5.31 | 1.15 | 7.63 | 7.16 | 2 |
| 556 | 898258 | AA598668 | 0.40 | 2.11 | 5.31 | 1.02 | 7.20 | 7.71 | 2 |
| 557 | 261714 | H98822 | 2.57 | 13.64 | 5.30 | 6.09 | 5.91 | 3.90 | 2 |
| 558 | 813997 | AA455652 | 4.09 | 21.66 | 5.30 | 0.94 | 5.86 | 9.09 | 2 |
| 559 | 265102 | N21334 | 0.76 | 4.03 | 5.29 | 1.07 | 9.61 | 5.20 | 2 |
| 560 | 247089 | N57858 | 0.04 | 0.23 | 5.29 | 1.93 | 6.76 | 7.18 | 2 |
| 561 | 108395 | T77840 | 3.81 | 20.12 | 5.29 | 2.59 | 8.00 | 5.27 | 2 |
| 562 | 1055831 | AA628190 | 0.08 | 0.41 | 5.28 | 2.22 | 6.68 | 6.95 | 2 |
| 563 | 204299 | H59305 | 0.70 | 3.68 | 5.26 | 5.35 | 2.95 | 7.50 | 2 |
| 564 | 1505735 | AA879474 | 0.03 | 0.14 | 5.26 | 5.79 | 2.97 | 7.02 | 2 |
| 565 | 1048685 | AA620614 | 0.11 | 0.58 | 5.24 | 2.21 | 5.07 | 8.43 | 2 |
| 566 | 490753 | AA133166 | 8.60 | 44.95 | 5.23 | 1.93 | 7.32 | 6.43 | 2 |
| 567 | 594517 | AA169645 | 2.92 | 15.25 | 5.23 | 2.51 | 5.49 | 7.68 | 2 |
| 568 | 784126 | AA446748 | 0.07 | 0.35 | 5.19 | 2.95 | 5.99 | 6.64 | 2 |
| 569 | 845602 | AA644334 | 1.83 | 9.47 | 5.18 | 1.72 | 6.91 | 6.90 | 2 |
| 570 | 768146 | AA426561 | 0.10 | 0.51 | 5.17 | 1.23 | 9.14 | 5.15 | 2 |
| 571 | 490060 | AA136060 | 0.48 | 2.47 | 5.16 | 3.25 | 5.27 | 6.97 | 2 |
| 572 | 726658 | AA398218 | 0.01 | 0.05 | 5.15 | 7.12 | 1.00 | 7.33 | 2 |
| 573 | 415806 | W84774 | 0.01 | 0.06 | 5.15 | 6.70 | 0.80 | 7.94 | 2 |
| 574 | 278483 | N66132 | 6.20 | 31.82 | 5.13 | 4.34 | 5.12 | 5.93 | 2 |
| 575 | 1293016 | AA683338 | 0.04 | 0.19 | 5.11 | 1.68 | 5.30 | 8.34 | 2 |
| 576 | 448920 | AA777779 | 0.08 | 0.43 | 5.10 | 1.62 | 8.40 | 5.28 | 2 |
| 577 | 1500388 | AA885851 | 0.15 | 0.77 | 5.09 | 4.62 | 5.03 | 5.63 | 2 |
| 578 | 491524 | AA148505 | 6.40 | 32.60 | 5.09 | 1.14 | 9.02 | 5.12 | 2 |
| 579 | 155575 | R71689 | 0.39 | 1.98 | 5.09 | 2.35 | 5.29 | 7.63 | 2 |
| 580 | 1602209 | AA962541 | 3.26 | 16.55 | 5.07 | 6.08 | 8.82 | 0.32 | 2 |
| 581 | 739247 | AA421230 | 8.07 | 40.76 | 5.05 | 1.54 | 8.12 | 5.48 | 2 |
| 582 | 824109 | AA490605 | 0.04 | 0.22 | 5.04 | 0.82 | 8.65 | 5.67 | 2 |
| 583 | 243653 | N49899 | 0.69 | 3.45 | 5.02 | 1.63 | 5.51 | 7.92 | 2 |
| 584 | 825411 | AA504272 | 4.68 | 23.47 | 5.02 | 3.05 | 6.66 | 5.33 | 2 |
| 585 | 564803 | AA129552 | 0.91 | 4.55 | 5.01 | 6.00 | 3.76 | 5.26 | 2 |
| 586 | 206052 | H61552 | 0.91 | 4.54 | 4.99 | 0.91 | 8.28 | 5.79 | 2 |
| 587 | 504279 | AA149637 | 1.71 | 8.53 | 4.99 | 7.00 | 6.10 | 1.87 | 2 |
| 588 | 264162 | N20480 | 0.39 | 1.93 | 4.97 | 1.99 | 6.33 | 6.61 | 2 |
| 589 | 877767 | AA626784 | 0.15 | 0.75 | 4.96 | 5.36 | 5.47 | 4.03 | 2 |
| 590 | 34294 | R44346 | 1.28 | 6.34 | 4.95 | 0.98 | 8.07 | 5.79 | 2 |
| 591 | 186132 | H39560 | 0.01 | 0.05 | 4.94 | 8.10 | 5.74 | 0.97 | 2 |
| 592 | 784772 | AA478542 | 1.78 | 8.75 | 4.92 | 0.97 | 8.14 | 5.65 | 2 |
| 593 | 757206 | AA443969 | 2.34 | 11.53 | 4.92 | 3.32 | 5.83 | 5.61 | 2 |
| 594 | 147925 | R82041 | 0.06 | 0.30 | 4.91 | 1.91 | 6.08 | 6.75 | 2 |
| 595 | 277545 | N56982 | 0.43 | 2.11 | 4.90 | 1.20 | 6.72 | 6.79 | 2 |
| 596 | 563201 | AA114106 | 0.02 | 0.08 | 4.90 | 5.15 | 7.27 | 2.27 | 2 |
| 597 | 50114 | H16743 | 1.70 | 8.32 | 4.90 | 0.37 | 7.99 | 6.33 | 2 |
| 598 | 1504152 | AA904969 | 0.14 | 0.71 | 4.89 | 1.97 | 6.45 | 6.25 | 2 |
| 599 | 40721 | R55750 | 0.30 | 1.49 | 4.88 | 1.57 | 7.62 | 5.46 | 2 |
| 600 | 471598 | AA035384 | 7.11 | 34.71 | 4.88 | 2.09 | 5.39 | 7.16 | 2 |
| 601 | 767994 | AA418918 | 0.54 | 2.66 | 4.87 | 3.38 | 5.34 | 5.90 | 2 |
| 602 | 290560 | N62372 | 2.16 | 10.43 | 4.84 | 2.45 | 5.47 | 6.59 | 2 |
| 603 | 809508 | AA454554 | 0.01 | 0.05 | 4.83 | 8.44 | 5.04 | 1.00 | 2 |
| 604 | 502296 | AA156599 | 0.19 | 0.90 | 4.81 | 3.32 | 5.49 | 5.64 | 2 |
| 605 | 382451 | AA064627 | 2.91 | 13.99 | 4.80 | 0.71 | 8.39 | 5.32 | 2 |
| 606 | 32517 | R43271 | 0.01 | 0.05 | 4.80 | 7.91 | 5.50 | 1.00 | 2 |
| 607 | 838568 | AA456931 | 19.35 | 92.90 | 4.80 | 2.12 | 5.68 | 6.60 | 2 |
| 608 | 258966 | N31641 | 1.57 | 7.55 | 4.80 | 1.94 | 5.26 | 7.20 | 2 |
| 609 | 503715 | AA131526 | 5.14 | 24.62 | 4.79 | 2.51 | 6.24 | 5.62 | 2 |
| 610 | 154472 | R54846 | 0.72 | 3.44 | 4.77 | 1.60 | 6.79 | 5.93 | 2 |
| 611 | 204129 | H55915 | 0.35 | 1.68 | 4.75 | 1.18 | 6.82 | 6.25 | 2 |
| 612 | 826089 | AA521411 | 0.13 | 0.60 | 4.75 | 1.30 | 6.47 | 6.47 | 2 |
| 613 | 123255 | R00275 | 0.92 | 4.36 | 4.75 | 2.49 | 5.38 | 6.37 | 2 |
| 614 | 453005 | AA779165 | 3.47 | 16.44 | 4.74 | 5.66 | 0.08 | 8.48 | 2 |
| 615 | 460553 | AA700415 | 0.06 | 0.31 | 4.74 | 2.27 | 5.74 | 6.21 | 2 |
| 616 | 43826 | H05768 | 0.96 | 4.54 | 4.72 | 2.52 | 5.51 | 6.12 | 2 |
| 617 | 66406 | T66936 | 0.74 | 3.48 | 4.67 | 5.15 | 3.86 | 5.01 | 2 |
| 618 | 432732 | AA701587 | 0.03 | 0.12 | 4.67 | 6.88 | 1.33 | 5.80 | 2 |
| 619 | 882488 | AA676590 | 0.06 | 0.27 | 4.65 | 6.62 | 5.90 | 1.44 | 2 |
| 620 | 376764 | AA046321 | 0.36 | 1.69 | 4.65 | 5.09 | 5.23 | 3.64 | 2 |
| 621 | 46097 | H08796 | 0.39 | 1.82 | 4.65 | 5.94 | 2.63 | 5.38 | 2 |
| 622 | 809466 | AA443094 | 0.90 | 4.18 | 4.63 | 2.19 | 5.33 | 6.39 | 2 |
| 623 | 745192 | AA626847 | 1.12 | 5.17 | 4.62 | 2.36 | 5.63 | 5.88 | 2 |
| 624 | 595109 | AA173926 | 2.40 | 11.07 | 4.60 | 3.28 | 5.08 | 5.45 | 2 |
| 625 | 898083 | AA598797 | 3.37 | 15.49 | 4.59 | 2.32 | 5.20 | 6.25 | 2 |
| 626 | 796606 | AA460521 | 0.97 | 4.45 | 4.59 | 2.55 | 5.24 | 5.98 | 2 |
| 627 | 327245 | AA284291 | 0.98 | 4.50 | 4.59 | 2.99 | 5.72 | 5.05 | 2 |
| 628 | 744044 | AA629251 | 0.01 | 0.06 | 4.58 | 5.99 | 0.75 | 7.00 | 2 |
| 629 | 882355 | AA676296 | 2.64 | 12.03 | 4.56 | 6.34 | 5.06 | 2.28 | 2 |
| 630 | 261567 | H98655 | 2.99 | 13.59 | 4.54 | 1.15 | 6.62 | 5.85 | 2 |
| 631 | 824728 | AA488979 | 1.11 | 5.05 | 4.54 | 0.93 | 7.47 | 5.23 | 2 |
| 632 | 283208 | N51367 | 0.59 | 2.67 | 4.54 | 0.99 | 7.39 | 5.23 | 2 |
| 633 | 453229 | AA704858 | 0.08 | 0.38 | 4.54 | 1.08 | 5.53 | 7.00 | 2 |
| 634 | 132954 | R24451 | 0.65 | 2.96 | 4.53 | 2.21 | 5.84 | 5.54 | 2 |
| 635 | 588840 | AA157787 | 3.00 | 13.59 | 4.53 | 3.16 | 5.08 | 5.34 | 2 |
| 636 | 731031 | AA421270 | 8.95 | 40.40 | 4.52 | 2.12 | 6.30 | 5.12 | 2 |
| 637 | 1468466 | AA885096 | 0.60 | 2.71 | 4.50 | 5.38 | 2.55 | 5.56 | 2 |
| 638 | 206272 | H58542 | 1.42 | 6.39 | 4.50 | 1.27 | 5.28 | 6.95 | 2 |
| 639 | 75650 | T58434 | 1.01 | 4.53 | 4.49 | 2.26 | 6.17 | 5.02 | 2 |
| 640 | 257312 | N29624 | 0.36 | 1.60 | 4.48 | 0.90 | 6.98 | 5.57 | 2 |
| 641 | 814765 | AA454947 | 2.35 | 10.51 | 4.48 | 5.20 | 5.13 | 3.12 | 2 |
| 642 | 1048899 | AA778562 | 0.07 | 0.32 | 4.48 | 1.56 | 5.09 | 6.78 | 2 |
| 643 | 51700 | H22854 | 0.24 | 1.09 | 4.47 | 6.45 | 5.25 | 1.71 | 2 |
| 644 | 1466599 | AA883660 | 0.12 | 0.53 | 4.46 | 1.21 | 6.24 | 5.93 | 2 |
| 645 | 51328 | H20743 | 0.01 | 0.06 | 4.44 | 2.31 | 5.02 | 5.98 | 2 |
| 646 | 1504005 | AA904776 | 0.11 | 0.49 | 4.44 | 1.72 | 5.71 | 5.87 | 2 |
| 647 | 194811 | R89765 | 0.42 | 1.88 | 4.42 | 0.87 | 5.63 | 6.76 | 2 |
| 648 | 126239 | R06372 | 0.02 | 0.08 | 4.42 | 5.24 | 2.84 | 5.17 | 2 |
| 649 | 726858 | AA398355 | 1.71 | 7.57 | 4.42 | 7.74 | 0.22 | 5.29 | 2 |
| 650 | 110772 | T90621 | 13.07 | 57.53 | 4.40 | 2.19 | 5.60 | 5.41 | 2 |
| 651 | 758302 | AA404338 | 0.03 | 0.14 | 4.39 | 6.57 | 0.31 | 6.30 | 2 |
| 652 | 530545 | AA112979 | 0.19 | 0.84 | 4.38 | 2.44 | 5.46 | 5.23 | 2 |
| 653 | 1461715 | AA884382 | 0.17 | 0.73 | 4.35 | 2.38 | 5.14 | 5.54 | 2 |
| 654 | 745523 | AA626247 | 0.09 | 0.40 | 4.35 | 1.74 | 6.04 | 5.26 | 2 |
| 655 | 744911 | AA625791 | 0.10 | 0.42 | 4.34 | 1.92 | 5.25 | 5.86 | 2 |
| 656 | 784589 | AA443300 | 0.02 | 0.10 | 4.33 | 5.57 | 6.96 | 0.44 | 2 |
| 657 | 647816 | AA205320 | 0.25 | 1.07 | 4.32 | 0.99 | 5.51 | 6.47 | 2 |
| 658 | 80946 | T70122 | 5.27 | 22.74 | 4.32 | 1.50 | 5.60 | 5.84 | 2 |
| 659 | 757463 | AA437245 | 0.09 | 0.39 | 4.28 | 1.45 | 5.78 | 5.62 | 2 |
| 660 | 45514 | H08227 | 0.06 | 0.27 | 4.28 | 1.28 | 5.77 | 5.79 | 2 |
| 661 | 280909 | N50828 | 1.43 | 6.07 | 4.25 | 1.08 | 5.61 | 6.07 | 2 |
| 662 | 814906 | AA465692 | 2.68 | 11.37 | 4.24 | 1.91 | 5.44 | 5.36 | 2 |
| 663 | 272238 | N35579 | 2.10 | 8.82 | 4.19 | 1.25 | 5.55 | 5.76 | 2 |
| 664 | 42400 | R60949 | 1.37 | 5.73 | 4.17 | 5.58 | 1.07 | 5.87 | 2 |
| 665 | 281103 | N50935 | 2.75 | 11.43 | 4.16 | 1.40 | 5.50 | 5.58 | 2 |
| 666 | 1466824 | AA883755 | 0.09 | 0.36 | 4.16 | 1.38 | 5.73 | 5.36 | 2 |
| 667 | 505997 | AA708431 | 0.41 | 1.69 | 4.14 | 5.38 | 1.63 | 5.41 | 2 |
| 668 | 841287 | AA487206 | 2.36 | 9.76 | 4.13 | 0.84 | 6.05 | 5.49 | 2 |
| 689 | 126695 | R07012 | 0.14 | 0.57 | 4.12 | 1.58 | 5.14 | 5.65 | 2 |
| 670 | 858292 | AA633993 | 2.38 | 9.78 | 4.10 | 0.57 | 5.73 | 6.01 | 2 |
| 671 | 1469211 | AA862814 | 0.20 | 0.83 | 4.09 | 1.23 | 5.12 | 5.93 | 2 |
| 672 | 73659 | T54527 | 1.12 | 4.55 | 4.08 | 0.97 | 5.71 | 5.55 | 2 |
| 673 | 79726 | T62552 | 1.04 | 4.25 | 4.08 | 5.88 | 5.23 | 1.12 | 2 |
| 674 | 292388 | N79230 | 1.82 | 7.30 | 4.01 | 5.25 | 1.78 | 5.01 | 2 |
| 675 | 239661 | H79566 | 0.02 | 0.07 | 3.92 | 5.37 | 5.82 | 0.56 | 2 |
| 676 | 882483 | AA676598 | 2.77 | 10.81 | 3.91 | 1.13 | 5.04 | 5.56 | 2 |
| 677 | 249517 | H84915 | 0.76 | 2.97 | 3.90 | 1.12 | 5.12 | 5.46 | 2 |
| 678 | 247587 | N58136 | 0.02 | 0.09 | 3.90 | 5.46 | 5.69 | 0.55 | 2 |
| 679 | 193420 | H47089 | 0.40 | 1.56 | 3.87 | 1.01 | 5.60 | 5.01 | 2 |
| 680 | 487141 | AA045340 | 1.95 | 7.56 | 3.87 | 0.43 | 5.56 | 5.63 | 2 |

TABLE 2-12
Prostate Cancer Cell Lines vs. Normal Prostatic Epithelial Cell Line: Genes
upregulated 10 fold or more in at least 2 cancer cell lines.

| | | Gen Bank | | Ave Cancer | Fold(Ave | Fold | Fold | Fold | |
|---|---|---|---|---|---|---|---|---|---|
| | IMAGE | Accession | MPM1 | Cell Lines | Cell line/ | (MPM2/MP | (MPM271/M | (MPM272/M | # of Cancer |
| Order | Clone ID | Number | (PrEC) | Values | MPM1) | M1) | PM1) | PM1) | Cell Lines |
| 1 | 1343732 | AA725564 | 0.01 | 1.12 | 112.48 | 13.45 | 301.13 | 22.85 | 3 |
| 2 | 784296 | AA447079 | 0.01 | 0.66 | 65.30 | 45.84 | 106.60 | 43.45 | 3 |
| 3 | 796569 | AA460463 | 0.01 | 0.35 | 63.57 | 16.10 | 138.91 | 35.70 | 3 |
| 4 | 1558655 | AA976561 | 0.08 | 4.36 | 54.32 | 97.06 | 17.92 | 47.97 | 3 |
| 5 | 743016 | AA406036 | 0.01 | 0.50 | 50.47 | 15.70 | 123.80 | 11.93 | 3 |
| 6 | 33076 | R44048 | 0.80 | 36.21 | 45.35 | 56.36 | 29.19 | 50.51 | 3 |
| 7 | 626390 | AA189106 | 0.07 | 3.16 | 45.16 | 91.62 | 20.97 | 22.90 | 3 |
| 8 | 812170 | AA456035 | 0.01 | 0.30 | 37.18 | 16.25 | 80.79 | 14.51 | 3 |
| 9 | 50288 | H17888 | 0.01 | 0.32 | 36.79 | 12.25 | 87.45 | 10.68 | 3 |
| 10 | 487861 | AA045436 | 0.02 | 0.59 | 32.57 | 29.44 | 39.44 | 28.82 | 3 |
| 11 | 291057 | N72115 | 0.23 | 6.85 | 29.19 | 24.48 | 38.30 | 24.80 | 3 |
| 12 | 263014 | H99813 | 0.63 | 17.34 | 27.31 | 19.07 | 21.59 | 41.29 | 3 |
| 13 | 303180 | N92764 | 0.01 | 0.27 | 27.21 | 13.82 | 36.04 | 31.77 | 3 |
| 14 | 46896 | H09818 | 0.05 | 1.21 | 25.96 | 33.86 | 30.16 | 13.87 | 3 |
| 15 | 151184 | H02294 | 0.48 | 12.53 | 25.93 | 38.89 | 13.26 | 25.64 | 3 |
| 16 | 41595 | R59556 | 0.01 | 0.17 | 25.59 | 12.08 | 52.20 | 12.48 | 3 |
| 17 | 430092 | AA009840 | 0.40 | 10.00 | 24.80 | 34.18 | 13.61 | 26.60 | 3 |
| 18 | 1049033 | AA778675 | 0.38 | 9.30 | 24.51 | 46.38 | 16.26 | 10.88 | 3 |
| 19 | 884790 | AA629838 | 0.01 | 0.24 | 24.29 | 30.26 | 10.09 | 32.50 | 3 |
| 20 | 365149 | AA025142 | 0.16 | 3.52 | 22.24 | 38.20 | 16.96 | 11.55 | 3 |
| 21 | 51581 | H22824 | 0.01 | 0.25 | 20.42 | 18.18 | 18.45 | 24.62 | 3 |
| 22 | 1467161 | AA883187 | 0.02 | 0.31 | 18.24 | 10.20 | 24.35 | 20.16 | 3 |
| 23 | 488431 | AA047441 | 0.17 | 2.95 | 17.83 | 10.45 | 28.90 | 14.13 | 3 |
| 24 | 82869 | T69270 | 0.02 | 0.32 | 15.94 | 21.48 | 14.07 | 12.25 | 3 |
| 25 | 898286 | AA598974 | 1.21 | 16.52 | 13.65 | 13.47 | 11.86 | 15.62 | 3 |
| 26 | 42627 | R60995 | 0.01 | 9.14 | 660.46 | 2.32 | 61.14 | 1917.92 | 2 |
| 27 | 757165 | AA443950 | 0.01 | 5.71 | 591.67 | 7.33 | 66.37 | 1701.31 | 2 |
| 28 | 592707 | AA160606 | 0.01 | 1.66 | 234.30 | 8.10 | 680.66 | 14.13 | 2 |
| 29 | 781036 | AA446446 | 0.01 | 1.70 | 228.86 | 10.58 | 674.66 | 1.35 | 2 |
| 30 | 220851 | H95633 | 0.01 | 1.28 | 195.56 | 8.10 | 570.75 | 10.82 | 2 |
| 31 | 203878 | H56453 | 0.02 | 1.93 | 95.78 | 0.80 | 140.40 | 146.14 | 2 |
| 32 | 815794 | AA485214 | 0.08 | 6.83 | 85.65 | 0.89 | 150.46 | 105.59 | 2 |
| 33 | 795627 | AA459917 | 0.01 | 0.51 | 85.17 | 3.09 | 239.88 | 12.54 | 2 |
| 34 | 347182 | W80611 | 0.01 | 0.82 | 81.58 | 1.42 | 229.57 | 13.76 | 2 |
| 35 | 746152 | AA419486 | 0.08 | 5.16 | 65.49 | 0.87 | 95.72 | 99.88 | 2 |
| 36 | 346942 | W94289 | 0.01 | 0.61 | 61.20 | 3.73 | 168.10 | 11.76 | 2 |
| 37 | 131452 | R23270 | 0.45 | 26.93 | 59.79 | 104.24 | 4.69 | 70.45 | 2 |
| 38 | 684879 | AA251784 | 0.05 | 2.87 | 58.88 | 1.03 | 51.92 | 123.70 | 2 |
| 39 | 344589 | W73144 | 0.22 | 12.41 | 56.02 | 78.84 | 88.50 | 0.72 | 2 |
| 40 | 33827 | R44741 | 0.02 | 1.10 | 50.72 | 0.63 | 139.36 | 12.17 | 2 |
| 41 | 39442 | R51617 | 0.10 | 5.02 | 49.93 | 99.52 | 44.77 | 5.49 | 2 |
| 42 | 825740 | AA504844 | 0.26 | 13.02 | 49.67 | 0.91 | 56.58 | 91.51 | 2 |
| 43 | 128083 | R09747 | 0.01 | 0.44 | 44.34 | 25.03 | 101.07 | 6.93 | 2 |
| 44 | 757368 | AA437126 | 0.01 | 0.42 | 41.72 | 4.59 | 109.53 | 11.05 | 2 |
| 45 | 1367900 | AA810225 | 0.01 | 0.41 | 41.11 | 4.81 | 99.20 | 19.33 | 2 |
| 46 | 243181 | H94482 | 0.01 | 0.36 | 35.62 | 5.91 | 88.28 | 12.68 | 2 |
| 47 | 196544 | R91566 | 0.25 | 8.56 | 34.73 | 0.93 | 52.03 | 51.22 | 2 |
| 48 | 256911 | N30098 | 0.01 | 0.25 | 34.44 | 20.19 | 73.72 | 9.41 | 2 |
| 49 | 1030613 | AA608832 | 0.01 | 0.33 | 33.18 | 5.81 | 82.72 | 11.01 | 2 |
| 50 | 815861 | AA485052 | 0.26 | 7.89 | 30.35 | 0.77 | 34.54 | 55.73 | 2 |
| 51 | 1455835 | AA863292 | 0.01 | 0.40 | 29.16 | 9.10 | 30.24 | 48.14 | 2 |
| 52 | 108378 | T77729 | 0.01 | 0.41 | 29.13 | 7.73 | 36.47 | 43.19 | 2 |
| 53 | 1391682 | AA789328 | 0.02 | 0.44 | 28.83 | 5.99 | 65.78 | 14.72 | 2 |
| 54 | 53039 | R15740 | 0.01 | 0.27 | 27.34 | 0.74 | 70.25 | 11.03 | 2 |
| 55 | 796549 | AA460274 | 0.01 | 0.27 | 27.13 | 1.21 | 69.04 | 11.15 | 2 |
| 56 | 194318 | H50655 | 0.53 | 14.38 | 26.99 | 0.69 | 27.83 | 52.45 | 2 |
| 57 | 768056 | AA418903 | 0.01 | 0.16 | 26.24 | 7.36 | 35.03 | 36.33 | 2 |
| 58 | 280699 | N47445 | 0.55 | 14.15 | 25.90 | 2.28 | 11.75 | 63.66 | 2 |
| 59 | 38344 | R49555 | 0.01 | 0.24 | 25.84 | 5.18 | 58.23 | 14.11 | 2 |
| 60 | 283379 | N52767 | 0.02 | 0.39 | 25.65 | 0.59 | 66.04 | 10.31 | 2 |
| 61 | 430255 | AA010383 | 0.01 | 0.25 | 25.24 | 4.46 | 54.64 | 16.63 | 2 |
| 62 | 687972 | AA236986 | 0.01 | 0.17 | 25.09 | 3.61 | 60.05 | 11.62 | 2 |
| 63 | 357884 | W94486 | 0.01 | 0.25 | 24.95 | 2.32 | 41.17 | 31.35 | 2 |
| 64 | 172517 | H19826 | 1.00 | 24.91 | 24.86 | 2.27 | 26.82 | 45.50 | 2 |
| 65 | 251806 | H96647 | 0.01 | 0.24 | 24.50 | 1.76 | 60.48 | 11.25 | 2 |
| 66 | 824025 | AA490945 | 0.31 | 7.41 | 24.24 | 0.95 | 28.65 | 43.13 | 2 |
| 67 | 23345 | R39191 | 0.01 | 0.16 | 23.97 | 44.37 | 17.55 | 9.98 | 2 |
| 68 | 825606 | AA504625 | 0.15 | 3.51 | 23.68 | 0.87 | 21.91 | 48.25 | 2 |
| 69 | 284714 | N63057 | 0.01 | 0.16 | 23.01 | 8.11 | 34.50 | 26.43 | 2 |
| 70 | 81050 | T70198 | 0.01 | 0.23 | 23.00 | 1.00 | 42.67 | 25.34 | 2 |
| 71 | 855910 | AA630328 | 0.01 | 0.16 | 22.37 | 4.98 | 30.37 | 31.76 | 2 |
| 72 | 34204 | R44936 | 0.17 | 3.74 | 22.30 | 3.82 | 51.95 | 11.14 | 2 |
| 73 | 824270 | AA491261 | 0.17 | 3.65 | 21.71 | 0.88 | 10.05 | 54.20 | 2 |
| 74 | 46843 | H10072 | 0.01 | 0.14 | 21.70 | 39.83 | 1.50 | 23.77 | 2 |
| 75 | 79000 | T61938 | 1.90 | 40.77 | 21.44 | 37.52 | 23.72 | 3.08 | 2 |
| 76 | 259884 | N32904 | 0.91 | 19.16 | 20.97 | 4.40 | 10.88 | 47.62 | 2 |
| 77 | 270975 | N32542 | 0.01 | 0.20 | 20.40 | 5.36 | 33.42 | 22.42 | 2 |
| 78 | 686552 | AA255954 | 0.30 | 6.19 | 20.39 | 0.93 | 42.76 | 17.48 | 2 |
| 79 | 51916 | H22563 | 0.12 | 2.46 | 20.27 | 20.94 | 30.57 | 9.30 | 2 |
| 80 | 199337 | R95684 | 0.20 | 3.94 | 20.04 | 1.29 | 30.54 | 28.27 | 2 |
| 81 | 795831 | AA461508 | 0.01 | 0.20 | 19.97 | 1.00 | 38.72 | 20.17 | 2 |
| 82 | 197265 | R86970 | 0.01 | 0.20 | 19.50 | 2.79 | 10.31 | 45.41 | 2 |
| 83 | 40010 | R54036 | 0.01 | 0.19 | 19.50 | 7.04 | 11.18 | 40.27 | 2 |
| 84 | 162310 | H28091 | 0.17 | 3.30 | 19.47 | 2.35 | 30.81 | 25.26 | 2 |
| 85 | 80338 | T65736 | 1.11 | 21.01 | 18.93 | 41.41 | 14.31 | 1.08 | 2 |
| 86 | 39722 | R54492 | 0.01 | 0.17 | 18.85 | 3.39 | 10.12 | 43.06 | 2 |
| 87 | 489076 | AA057195 | 0.02 | 0.35 | 18.01 | 4.49 | 38.83 | 10.71 | 2 |
| 88 | 361899 | AA001376 | 0.01 | 0.17 | 17.44 | 1.97 | 34.15 | 16.22 | 2 |
| 89 | 435099 | AA701315 | 0.01 | 0.21 | 17.36 | 7.52 | 33.82 | 10.73 | 2 |
| 90 | 67440 | T49355 | 1.96 | 33.78 | 17.25 | 25.95 | 8.91 | 16.88 | 2 |
| 91 | 1410444 | AA857163 | 0.06 | 1.06 | 17.19 | 1.87 | 37.48 | 12.21 | 2 |
| 92 | 280155 | N47012 | 0.04 | 0.63 | 17.15 | 5.64 | 11.78 | 34.04 | 2 |
| 93 | 454083 | AA676998 | 0.01 | 0.17 | 16.89 | 8.16 | 10.67 | 31.84 | 2 |
| 94 | 756629 | AA481481 | 0.01 | 0.11 | 16.78 | 14.65 | 1.51 | 34.18 | 2 |
| 95 | 358990 | W92263 | 0.01 | 0.16 | 16.35 | 10.47 | 5.09 | 33.48 | 2 |
| 96 | 357373 | W93717 | 0.70 | 11.31 | 16.17 | 5.81 | 15.89 | 26.82 | 2 |
| 97 | 825726 | AA504838 | 0.19 | 2.94 | 15.84 | 2.63 | 16.52 | 28.36 | 2 |
| 98 | 746232 | AA417713 | 0.38 | 5.98 | 15.64 | 0.69 | 21.09 | 25.15 | 2 |
| 99 | 487766 | AA045175 | 0.02 | 0.30 | 15.57 | 4.69 | 22.78 | 19.22 | 2 |
| 100 | 811766 | AA463445 | 0.26 | 3.91 | 15.03 | 1.53 | 28.43 | 15.13 | 2 |
| 101 | 51599 | H18932 | 0.26 | 3.94 | 14.95 | 0.42 | 26.10 | 18.34 | 2 |
| 102 | 250069 | H97140 | 0.02 | 0.35 | 14.92 | 5.14 | 24.61 | 15.00 | 2 |
| 103 | 28298 | R40434 | 0.02 | 0.35 | 14.61 | 5.11 | 16.46 | 22.27 | 2 |
| 104 | 785293 | AA476543 | 0.11 | 1.64 | 14.41 | 24.69 | 3.02 | 15.54 | 2 |
| 105 | 770860 | AA434388 | 0.01 | 0.14 | 14.40 | 1.00 | 18.93 | 23.25 | 2 |
| 106 | 1461609 | AA883800 | 0.25 | 3.51 | 14.24 | 10.07 | 4.19 | 28.45 | 2 |
| 107 | 418004 | W90705 | 0.30 | 4.17 | 13.89 | 2.99 | 11.04 | 27.66 | 2 |
| 108 | 41406 | R56149 | 0.01 | 0.13 | 13.76 | 17.85 | 18.97 | 4.48 | 2 |
| 109 | 1035796 | AA628867 | 1.14 | 15.63 | 13.71 | 10.64 | 9.76 | 20.74 | 2 |
| 110 | 26806 | R37738 | 0.10 | 1.31 | 13.68 | 2.42 | 26.44 | 12.18 | 2 |
| 111 | 502003 | AA128617 | 0.01 | 0.17 | 13.58 | 7.33 | 15.99 | 17.41 | 2 |
| 112 | 771004 | AA427719 | 0.29 | 3.95 | 13.50 | 0.64 | 21.58 | 18.27 | 2 |
| 113 | 415089 | W93379 | 0.65 | 8.63 | 13.24 | 8.23 | 12.04 | 19.45 | 2 |
| 114 | 824074 | AA491227 | 0.02 | 0.26 | 13.22 | 3.39 | 18.08 | 18.18 | 2 |
| 115 | 51103 | H19217 | 0.02 | 0.23 | 13.14 | 14.72 | 17.85 | 6.86 | 2 |
| 116 | 284288 | N52193 | 0.01 | 0.08 | 13.00 | 10.97 | 1.54 | 26.50 | 2 |
| 117 | 361122 | AA017379 | 0.01 | 0.13 | 12.88 | 0.65 | 12.28 | 25.70 | 2 |
| 118 | 244050 | N34042 | 0.01 | 0.17 | 12.88 | 5.65 | 21.72 | 11.26 | 2 |
| 119 | 795446 | AA453616 | 0.01 | 0.13 | 12.56 | 22.57 | 14.55 | 0.58 | 2 |
| 120 | 1049185 | AA620697 | 0.76 | 9.44 | 12.39 | 10.40 | 0.39 | 26.39 | 2 |
| 121 | 364352 | AA022496 | 0.01 | 0.12 | 12.38 | 1.96 | 14.19 | 20.98 | 2 |
| 122 | 47264 | H10713 | 0.30 | 3.72 | 12.36 | 2.11 | 23.87 | 11.09 | 2 |
| 123 | 686172 | AA262211 | 1.18 | 14.39 | 12.18 | 5.65 | 13.80 | 17.09 | 2 |
| 124 | 1049079 | AA778717 | 0.03 | 0.31 | 12.10 | 3.28 | 15.47 | 17.55 | 2 |
| 125 | 195845 | R92201 | 0.14 | 1.69 | 12.02 | 1.00 | 14.95 | 20.10 | 2 |
| 126 | 451649 | AA706901 | 1.40 | 16.82 | 11.98 | 4.34 | 18.70 | 12.88 | 2 |
| 127 | 739450 | AA477227 | 1.99 | 23.53 | 11.82 | 2.08 | 17.13 | 16.24 | 2 |
| 128 | 46461 | H09940 | 0.33 | 3.89 | 11.80 | 3.09 | 13.18 | 19.11 | 2 |
| 129 | 1030798 | AA609002 | 0.03 | 0.31 | 11.73 | 10.15 | 8.33 | 16.72 | 2 |
| 130 | 591157 | AA161188 | 0.02 | 0.18 | 11.71 | 0.58 | 20.54 | 14.01 | 2 |
| 131 | 33839 | R44816 | 0.96 | 11.24 | 11.67 | 3.60 | 19.31 | 12.09 | 2 |
| 132 | 357138 | W93523 | 0.02 | 0.20 | 11.63 | 10.87 | 20.61 | 3.40 | 2 |
| 133 | 366971 | AA026682 | 2.38 | 27.64 | 11.61 | 3.65 | 10.18 | 20.99 | 2 |
| 134 | 592728 | AA160670 | 0.03 | 0.34 | 11.17 | 0.33 | 16.85 | 16.34 | 2 |
| 135 | 193333 | H48070 | 0.36 | 3.90 | 10.90 | 0.92 | 17.96 | 13.80 | 2 |
| 136 | 753428 | AA410434 | 0.57 | 6.12 | 10.84 | 0.51 | 15.37 | 16.62 | 2 |
| 137 | 298417 | N74131 | 0.03 | 0.31 | 10.83 | 2.09 | 20.21 | 10.20 | 2 |
| 138 | 795840 | AA461513 | 0.01 | 0.06 | 10.81 | 4.64 | 15.78 | 12.02 | 2 |
| 139 | 257955 | N30751 | 0.86 | 9.32 | 10.79 | 2.05 | 10.22 | 20.10 | 2 |
| 140 | 788209 | AA453433 | 0.01 | 0.09 | 10.70 | 11.07 | 8.10 | 12.92 | 2 |
| 141 | 204483 | H58234 | 1.27 | 13.51 | 10.64 | 11.48 | 13.18 | 7.26 | 2 |
| 142 | 712401 | AA281784 | 0.08 | 0.87 | 10.32 | 0.88 | 14.48 | 15.60 | 2 |
| 143 | 277621 | N49389 | 0.54 | 5.55 | 10.21 | 0.77 | 14.23 | 15.64 | 2 |
| 144 | 590640 | AA158035 | 0.01 | 0.12 | 10.11 | 11.51 | 17.15 | 1.65 | 2 |
| 145 | 197651 | R94504 | 0.25 | 2.53 | 10.07 | 0.93 | 11.85 | 17.42 | 2 |
| 146 | 855391 | AA664007 | 0.23 | 2.29 | 9.95 | 1.40 | 12.15 | 16.30 | 2 |
| 147 | 745490 | AA625979 | 0.11 | 1.05 | 9.95 | 12.71 | 3.07 | 14.06 | 2 |
| 148 | 725877 | AA292226 | 0.50 | 4.94 | 9.89 | 0.67 | 13.69 | 15.31 | 2 |
| 149 | 266135 | N21633 | 0.39 | 3.82 | 9.87 | 1.10 | 17.62 | 10.88 | 2 |
| 150 | 433567 | AA701652 | 0.02 | 0.16 | 9.57 | 11.04 | 10.28 | 7.37 | 2 |
| 151 | 35804 | R46000 | 0.08 | 0.77 | 9.53 | 2.05 | 15.63 | 10.89 | 2 |
| 152 | 280375 | N47113 | 1.12 | 10.61 | 9.52 | 11.98 | 10.79 | 5.79 | 2 |
| 153 | 303099 | N90779 | 0.15 | 1.40 | 9.48 | 5.53 | 11.42 | 11.50 | 2 |
| 154 | 630013 | AA219060 | 0.15 | 1.40 | 9.48 | 1.15 | 11.85 | 15.44 | 2 |
| 155 | 320495 | W16659 | 0.01 | 0.09 | 9.47 | 10.45 | 1.00 | 16.97 | 2 |
| 156 | 700792 | AA284072 | 0.01 | 0.06 | 9.34 | 11.43 | 1.54 | 15.06 | 2 |
| 157 | 281162 | N50962 | 0.02 | 0.22 | 9.31 | 0.97 | 12.53 | 14.44 | 2 |
| 158 | 43733 | H04789 | 0.11 | 1.03 | 9.24 | 10.06 | 12.44 | 5.24 | 2 |
| 159 | 461488 | AA705047 | 0.10 | 0.97 | 9.22 | 12.49 | 10.86 | 4.33 | 2 |
| 160 | 781047 | AA446462 | 1.08 | 9.89 | 9.17 | 4.58 | 11.63 | 11.30 | 2 |
| 161 | 199577 | R96579 | 0.27 | 2.44 | 9.12 | 0.81 | 13.04 | 13.51 | 2 |
| 162 | 141726 | R69584 | 0.03 | 0.26 | 9.09 | 3.46 | 10.73 | 13.08 | 2 |
| 163 | 320865 | W44766 | 0.03 | 0.26 | 9.07 | 12.50 | 0.36 | 14.35 | 2 |
| 164 | 266259 | N26515 | 0.35 | 3.21 | 9.06 | 1.65 | 10.87 | 14.67 | 2 |
| 165 | 594758 | AA172056 | 1.30 | 11.71 | 9.03 | 12.54 | 13.45 | 1.10 | 2 |
| 166 | 951241 | AA620485 | 1.98 | 17.81 | 8.98 | 10.05 | 0.30 | 16.60 | 2 |
| 167 | 342640 | W68220 | 0.67 | 5.97 | 8.87 | 11.72 | 2.96 | 11.94 | 2 |
| 168 | 897546 | AA496998 | 0.01 | 0.09 | 8.56 | 10.33 | 1.00 | 14.35 | 2 |
| 169 | 324717 | W47364 | 1.13 | 9.51 | 8.39 | 1.45 | 12.46 | 11.27 | 2 |
| 170 | 825416 | AA504265 | 0.40 | 3.36 | 8.39 | 1.30 | 11.51 | 12.36 | 2 |
| 171 | 380884 | AA058576 | 0.18 | 1.49 | 8.21 | 2.23 | 11.14 | 11.26 | 2 |
| 172 | 451907 | AA706968 | 2.08 | 15.11 | 7.27 | 11.07 | 0.08 | 10.67 | 2 |

TABLE 3-1

| Sequence # | Accession # | Database |
|---|---|---|
| Sequence 1 | A21185 | GENBANK |
| Sequence 2 | AB000509 | GENBANK |
| Sequence 3 | AB002329 | GENBANK |
| Sequence 4 | AB002357 | GENBANK |
| Sequence 5 | AB002366 | GENBANK |
| Sequence 6 | AB002387 | GENBANK |
| Sequence 7 | AB004304 | GENBANK |
| Sequence 8 | AB004788 | GENBANK |
| Sequence 9 | AB004903 | GENBANK |
| Sequence 10 | AB007870 | GENBANK |
| Sequence 11 | AB007899 | GENBANK |
| Sequence 12 | AB007930 | GENBANK |
| Sequence 13 | AB007941 | GENBANK |
| Sequence 14 | AB007956 | GENBANK |
| Sequence 15 | AB007957 | GENBANK |
| Sequence 16 | AB007960 | GENBANK |
| Sequence 17 | AB009284 | GENBANK |
| Sequence 18 | AB011108 | GENBANK |
| Sequence 19 | AB011132 | GENBANK |
| Sequence 20 | AB011140 | GENBANK |
| Sequence 21 | AB011155 | GENBANK |
| Sequence 22 | AB011159 | GENBANK |
| Sequence 23 | AB011164 | GENBANK |
| Sequence 24 | AB011472 | GENBANK |
| Sequence 25 | AB012130 | GENBANK |
| Sequence 26 | AB012910 | GENBANK |
| Sequence 27 | AB014521 | GENBANK |
| Sequence 28 | AB014589 | GENBANK |
| Sequence 29 | AB017007 | GENBANK |
| Sequence 30 | AB018268 | GENBANK |
| Sequence 31 | AB018305 | GENBANK |
| Sequence 32 | AB018327 | GENBANK |
| Sequence 33 | AB018344 | GENBANK |
| Sequence 34 | AB018351 | GENBANK |
| Sequence 35 | AB019568 | GENBANK |
| Sequence 36 | AB020637 | GENBANK |
| Sequence 37 | AB020669 | GENBANK |
| Sequence 38 | AB020680 | GENBANK |
| Sequence 39 | AB020682 | GENBANK |
| Sequence 40 | AB020692 | GENBANK |
| Sequence 41 | AB020723 | GENBANK |
| Sequence 42 | AB020981 | GENBANK |
| Sequence 43 | AB021288 | GENBANK |
| Sequence 44 | AB023153 | GENBANK |
| Sequence 45 | AB023206 | GENBANK |
| Sequence 46 | AB023227 | GENBANK |
| Sequence 47 | AB024704 | GENBANK |
| Sequence 48 | AB028624 | GENBANK |
| Sequence 49 | AB029000 | GENBANK |
| Sequence 50 | AB029004 | GENBANK |
| Sequence 51 | AB033020 | GENBANK |
| Sequence 52 | AB033048 | GENBANK |
| Sequence 53 | AB033114 | GENBANK |
| Sequence 54 | AD001528 | GENBANK |
| Sequence 55 | AF000986 | GENBANK |
| Sequence 56 | AF001893 | GENBANK |
| Sequence 57 | AF002715 | GENBANK |
| Sequence 58 | AF003837 | GENBANK |
| Sequence 59 | AF004561 | GENBANK |
| Sequence 60 | AF006085 | GENBANK |
| Sequence 61 | AF007791 | GENBANK |
| Sequence 62 | AF008442 | GENBANK |
| Sequence 63 | AF010235 | GENBANK |
| Sequence 64 | AF013988 | GENBANK |
| Sequence 65 | AF016266 | GENBANK |
| Sequence 66 | AF016270 | GENBANK |
| Sequence 67 | AF019226 | GENBANK |
| Sequence 68 | AF023259 | GENBANK |
| Sequence 69 | AF025840 | GENBANK |
| Sequence 70 | AF025998 | GENBANK |
| Sequence 71 | AF026939 | GENBANK |
| Sequence 72 | AF027824 | GENBANK |
| Sequence 73 | AF029890 | GENBANK |
| Sequence 74 | AF033095 | GENBANK |
| Sequence 75 | AF034607 | GENBANK |
| Sequence 76 | AF035296 | GENBANK |
| Sequence 77 | AF035298 | GENBANK |
| Sequence 78 | AF035309 | GENBANK |
| Sequence 79 | AF038404 | GENBANK |
| Sequence 80 | AF038957 | GENBANK |
| Sequence 81 | AF038960 | GENBANK |
| Sequence 82 | AF039703 | GENBANK |
| Sequence 83 | AF039918 | GENBANK |
| Sequence 84 | AF042331 | GENBANK |
| Sequence 85 | AF044588 | GENBANK |
| Sequence 86 | AF045167 | GENBANK |
| Sequence 87 | AF045184 | GENBANK |
| Sequence 88 | AF047020 | GENBANK |
| Sequence 89 | AF047472 | GENBANK |
| Sequence 90 | AF048977 | GENBANK |
| Sequence 91 | AF050171 | GENBANK |
| Sequence 92 | AF052124 | GENBANK |
| Sequence 93 | AF052164 | GENBANK |
| Sequence 94 | AF052182 | GENBANK |
| Sequence 95 | AF053470 | GENBANK |
| Sequence 96 | AF054179 | GENBANK |
| Sequence 97 | AF054187 | GENBANK |
| Sequence 98 | AF054838 | GENBANK |
| Sequence 99 | AF054990 | GENBANK |
| Sequence 100 | AF055012 | GENBANK |
| Sequence 101 | AF057160 | GENBANK |
| Sequence 102 | AF061258 | GENBANK |
| Sequence 103 | AF061938 | GENBANK |
| Sequence 104 | AF067656 | GENBANK |
| Sequence 105 | AF067972 | GENBANK |
| Sequence 106 | AF068846 | GENBANK |
| Sequence 107 | AF069601 | GENBANK |
| Sequence 108 | AF070523 | GENBANK |
| Sequence 109 | AF070555 | GENBANK |
| Sequence 110 | AF070561 | GENBANK |
| Sequence 111 | AF070655 | GENBANK |
| Sequence 112 | AF070657 | GENBANK |
| Sequence 113 | AF070664 | GENBANK |
| Sequence 114 | AF071202 | GENBANK |
| Sequence 115 | AF071593 | GENBANK |
| Sequence 116 | AF077030 | GENBANK |
| Sequence 117 | AF077034 | GENBANK |
| Sequence 118 | AF077367 | GENBANK |
| Sequence 119 | AF083441 | GENBANK |
| Sequence 120 | AF083470 | GENBANK |
| Sequence 121 | AF085845 | GENBANK |
| Sequence 122 | AF086003 | GENBANK |
| Sequence 123 | AF086218 | GENBANK |
| Sequence 124 | AF086336 | GENBANK |
| Sequence 125 | AF086431 | GENBANK |
| Sequence 126 | AF086495 | GENBANK |
| Sequence 127 | AF086517 | GENBANK |
| Sequence 128 | AF086557 | GENBANK |
| Sequence 129 | AF087020 | GENBANK |
| Sequence 130 | AF087573 | GENBANK |
| Sequence 131 | AF088055 | GENBANK |
| Sequence 132 | AF091089 | GENBANK |
| Sequence 133 | AF093118 | GENBANK |
| Sequence 134 | AF100615 | GENBANK |
| Sequence 135 | AF100755 | GENBANK |
| Sequence 136 | AF100756 | GENBANK |
| Sequence 137 | AF101051 | GENBANK |
| Sequence 138 | AF103774 | GENBANK |
| Sequence 139 | AF103907 | GENBANK |
| Sequence 140 | AF106681 | GENBANK |
| Sequence 141 | AF110643 | GENBANK |
| Sequence 142 | AF112152 | GENBANK |
| Sequence 143 | AF114471 | GENBANK |
| Sequence 144 | AF119297 | GENBANK |
| Sequence 145 | AF124438 | GENBANK |
| Sequence 146 | AF128527 | GENBANK |
| Sequence 147 | AF134159 | GENBANK |
| Sequence 148 | AF139461 | GENBANK |
| Sequence 149 | AF147380 | GENBANK |
| Sequence 150 | AF151103 | GENBANK |
| Sequence 151 | AF151840 | GENBANK |
| Sequence 152 | AF151903 | GENBANK |
| Sequence 153 | AF152306 | GENBANK |
| Sequence 154 | AF153191 | GENBANK |
| Sequence 155 | AF155099 | GENBANK |
| Sequence 156 | AF155832 | GENBANK |
| Sequence 157 | AF159056 | GENBANK |
| Sequence 158 | AF176574 | GENBANK |
| Sequence 159 | AF182645 | GENBANK |
| Sequence 160 | AF188745 | GENBANK |
| Sequence 161 | AF192979 | GENBANK |
| Sequence 162 | AJ001306 | GENBANK |
| Sequence 163 | AJ004913 | GENBANK |
| Sequence 164 | AJ004955 | GENBANK |
| Sequence 165 | AJ006470 | GENBANK |
| Sequence 166 | AJ006834 | GENBANK |
| Sequence 167 | AJ007398 | GENBANK |
| Sequence 168 | AJ010442 | GENBANK |
| Sequence 169 | AJ010444 | GENBANK |
| Sequence 170 | AJ012499 | GENBANK |
| Sequence 171 | AJ130733 | GENBANK |
| Sequence 172 | AJ224172 | GENBANK |
| Sequence 173 | AJ227918 | GENBANK |
| Sequence 174 | AJ228139 | GENBANK |
| Sequence 175 | AJ238095 | GENBANK |
| Sequence 176 | AJ250042 | GENBANK |
| Sequence 177 | AL049227 | GENBANK |
| Sequence 178 | AL049246 | GENBANK |
| Sequence 179 | AL049471 | GENBANK |
| Sequence 180 | AL049799 | GENBANK |
| Sequence 181 | AL049932 | GENBANK |
| Sequence 182 | AL049941 | GENBANK |
| Sequence 183 | AL049942 | GENBANK |
| Sequence 184 | AL049969 | GENBANK |
| Sequence 185 | AL049987 | GENBANK |
| Sequence 186 | AL050041 | GENBANK |
| Sequence 187 | AL050141 | GENBANK |
| Sequence 188 | AL050161 | GENBANK |
| Sequence 189 | AL050184 | GENBANK |
| Sequence 190 | AL050198 | GENBANK |
| Sequence 191 | AL080172 | GENBANK |
| Sequence 192 | AL080234 | GENBANK |
| Sequence 193 | AL096719 | GENBANK |
| Sequence 194 | AL110144 | GENBANK |
| Sequence 195 | AL110183 | GENBANK |
| Sequence 196 | AL110197 | GENBANK |
| Sequence 197 | AL110206 | GENBANK |
| Sequence 198 | AL110212 | GENBANK |
| Sequence 199 | AL117237 | GENBANK |
| Sequence 200 | AL117427 | GENBANK |
| Sequence 201 | AL117429 | GENBANK |
| Sequence 202 | AL117505 | GENBANK |
| Sequence 203 | AL117534 | GENBANK |
| Sequence 204 | AL117554 | GENBANK |
| Sequence 205 | AL117576 | GENBANK |
| Sequence 206 | AL117609 | GENBANK |
| Sequence 207 | AL117643 | GENBANK |
| Sequence 208 | AL133000 | GENBANK |
| Sequence 209 | D00015 | GENBANK |
| Sequence 210 | D00017 | GENBANK |
| Sequence 211 | D13641 | GENBANK |
| Sequence 212 | D14041 | GENBANK |
| Sequence 213 | D14696 | GENBANK |
| Sequence 214 | D16431 | GENBANK |
| Sequence 215 | D25215 | GENBANK |
| Sequence 216 | D26488 | GENBANK |
| Sequence 217 | D28358 | GENBANK |
| Sequence 218 | D28420 | GENBANK |
| Sequence 219 | D28482 | GENBANK |
| Sequence 220 | D29954 | GENBANK |
| Sequence 221 | D29958 | GENBANK |
| Sequence 222 | D30037 | GENBANK |
| Sequence 223 | D31839 | GENBANK |
| Sequence 224 | D37965 | GENBANK |
| Sequence 225 | D38551 | GENBANK |
| Sequence 226 | D38583 | GENBANK |
| Sequence 227 | D42040 | GENBANK |
| Sequence 228 | D43950 | GENBANK |
| Sequence 229 | D50371 | GENBANK |
| Sequence 230 | D63486 | GENBANK |
| Sequence 231 | D63874 | GENBANK |
| Sequence 232 | D80009 | GENBANK |
| Sequence 233 | D83032 | GENBANK |
| Sequence 234 | D83197 | GENBANK |
| Sequence 235 | D83485 | GENBANK |
| Sequence 236 | D84557 | GENBANK |
| Sequence 237 | D86228 | GENBANK |
| Sequence 238 | D86322 | GENBANK |
| Sequence 239 | D87437 | GENBANK |
| Sequence 240 | D87444 | GENBANK |
| Sequence 241 | D87466 | GENBANK |
| Sequence 242 | D87666 | GENBANK |
| Sequence 243 | D87667 | GENBANK |
| Sequence 244 | D87685 | GENBANK |
| Sequence 245 | D87930 | GENBANK |
| Sequence 246 | D89053 | GENBANK |
| Sequence 247 | D89667 | GENBANK |
| Sequence 248 | D89937 | GENBANK |
| Sequence 249 | D90359 | GENBANK |
| Sequence 250 | D90427 | GENBANK |
| Sequence 251 | E01497 | GENBANK |
| Sequence 252 | E01956 | GENBANK |
| Sequence 253 | E01979 | GENBANK |
| Sequence 254 | E02628 | GENBANK |
| Sequence 255 | E02822 | GENBANK |
| Sequence 256 | E02823 | GENBANK |
| Sequence 257 | E06721 | GENBANK |
| Sequence 258 | J00194 | GENBANK |
| Sequence 259 | J03040 | GENBANK |
| Sequence 260 | J03209 | GENBANK |
| Sequence 261 | J03250 | GENBANK |
| Sequence 262 | J03464 | GENBANK |
| Sequence 263 | J03779 | GENBANK |
| Sequence 264 | J03799 | GENBANK |
| Sequence 265 | J04164 | GENBANK |
| Sequence 266 | J04621 | GENBANK |
| Sequence 267 | J04970 | GENBANK |
| Sequence 268 | J05021 | GENBANK |
| Sequence 269 | J05032 | GENBANK |
| Sequence 270 | K01911 | GENBANK |
| Sequence 271 | K02765 | GENBANK |
| Sequence 272 | L00160 | GENBANK |
| Sequence 273 | L01439 | GENBANK |
| Sequence 274 | L02426 | GENBANK |
| Sequence 275 | L05425 | GENBANK |
| Sequence 276 | L06133 | GENBANK |
| Sequence 277 | L08441 | GENBANK |
| Sequence 278 | L10678 | GENBANK |
| Sequence 279 | L10910 | GENBANK |
| Sequence 280 | L11066 | GENBANK |
| Sequence 281 | L11932 | GENBANK |
| Sequence 282 | L13210 | GENBANK |
| Sequence 283 | L13385 | GENBANK |
| Sequence 284 | L13773 | GENBANK |
| Sequence 285 | L13799 | GENBANK |
| Sequence 286 | L15702 | GENBANK |
| Sequence 287 | L19161 | GENBANK |
| Sequence 288 | L19605 | GENBANK |
| Sequence 289 | L19872 | GENBANK |
| Sequence 290 | L20941 | GENBANK |
| Sequence 291 | L22569 | GENBANK |
| Sequence 292 | L28010 | GENBANK |
| Sequence 293 | L33930 | GENBANK |
| Sequence 294 | L36642 | GENBANK |
| Sequence 295 | L38941 | GENBANK |
| Sequence 296 | L38961 | GENBANK |
| Sequence 297 | L39833 | GENBANK |
| Sequence 298 | L48984 | GENBANK |
| Sequence 299 | M10119 | GENBANK |
| Sequence 300 | M11119 | GENBANK |
| Sequence 301 | M12670 | GENBANK |
| Sequence 302 | M13231 | GENBANK |
| Sequence 303 | M13692 | GENBANK |
| Sequence 304 | M15661 | GENBANK |
| Sequence 305 | M15885 | GENBANK |
| Sequence 306 | M15887 | GENBANK |
| Sequence 307 | M16247 | GENBANK |
| Sequence 308 | M16342 | GENBANK |
| Sequence 309 | M16660 | GENBANK |
| Sequence 310 | M16768 | GENBANK |
| Sequence 311 | M17324 | GENBANK |
| Sequence 312 | M17517 | GENBANK |
| Sequence 313 | M17885 | GENBANK |
| Sequence 314 | M18366 | GENBANK |
| Sequence 315 | M21302 | GENBANK |
| Sequence 316 | M22382 | GENBANK |
| Sequence 317 | M22918 | GENBANK |
| Sequence 318 | M22920 | GENBANK |
| Sequence 319 | M24194 | GENBANK |
| Sequence 320 | M24594 | GENBANK |
| Sequence 321 | M24902 | GENBANK |
| Sequence 322 | M25246 | GENBANK |
| Sequence 323 | M26663 | GENBANK |
| Sequence 324 | M27332 | GENBANK |
| Sequence 325 | M27334 | GENBANK |
| Sequence 326 | M27826 | GENBANK |
| Sequence 327 | M28211 | GENBANK |
| Sequence 328 | M28372 | GENBANK |
| Sequence 329 | M29064 | GENBANK |
| Sequence 330 | M29550 | GENBANK |
| Sequence 331 | M29870 | GENBANK |
| Sequence 332 | M34840 | GENBANK |
| Sequence 333 | M36341 | GENBANK |
| Sequence 334 | M37583 | GENBANK |
| Sequence 335 | M55409 | GENBANK |
| Sequence 336 | M55542 | GENBANK |
| Sequence 337 | M55543 | GENBANK |
| Sequence 338 | M58485 | GENBANK |
| Sequence 339 | M59305 | GENBANK |
| Sequence 340 | M62840 | GENBANK |
| Sequence 341 | M63838 | GENBANK |
| Sequence 342 | M69181 | GENBANK |
| Sequence 343 | M74509 | GENBANK |
| Sequence 344 | M74558 | GENBANK |
| Sequence 345 | M74777 | GENBANK |
| Sequence 346 | M76180 | GENBANK |
| Sequence 347 | M77830 | GENBANK |
| Sequence 348 | M80902 | GENBANK |
| Sequence 349 | M83653 | GENBANK |
| Sequence 350 | M83738 | GENBANK |
| Sequence 351 | M83941 | GENBANK |
| Sequence 352 | M85164 | GENBANK |
| Sequence 353 | M87339 | GENBANK |
| Sequence 354 | M92439 | GENBANK |
| Sequence 355 | M94654 | GENBANK |
| Sequence 356 | S70154 | GENBANK |
| Sequence 357 | S71513 | GENBANK |
| Sequence 358 | S82240 | GENBANK |
| Sequence 359 | U02556 | GENBANK |
| Sequence 360 | U03271 | GENBANK |
| Sequence 361 | U07681 | GENBANK |
| Sequence 362 | U08815 | GENBANK |
| Sequence 363 | U09564 | GENBANK |
| Sequence 364 | U09848 | GENBANK |
| Sequence 365 | U10439 | GENBANK |
| Sequence 366 | U13616 | GENBANK |
| Sequence 367 | U14394 | GENBANK |
| Sequence 368 | U14510 | GENBANK |
| Sequence 369 | U16799 | GENBANK |
| Sequence 370 | U19557 | GENBANK |
| Sequence 371 | U24105 | GENBANK |
| Sequence 372 | U24704 | GENBANK |
| Sequence 373 | U28964 | GENBANK |
| Sequence 374 | U30521 | GENBANK |
| Sequence 375 | U34252 | GENBANK |
| Sequence 376 | U37143 | GENBANK |
| Sequence 377 | U37283 | GENBANK |
| Sequence 378 | U37519 | GENBANK |
| Sequence 379 | U38654 | GENBANK |
| Sequence 380 | U39360 | GENBANK |
| Sequence 381 | U39840 | GENBANK |
| Sequence 382 | U40671 | GENBANK |
| Sequence 383 | U41515 | GENBANK |
| Sequence 384 | U42594 | GENBANK |
| Sequence 385 | U51134 | GENBANK |
| Sequence 386 | U54558 | GENBANK |
| Sequence 387 | U54831 | GENBANK |
| Sequence 388 | U56255 | GENBANK |
| Sequence 389 | U58855 | GENBANK |
| Sequence 390 | U60975 | GENBANK |
| Sequence 391 | U62961 | GENBANK |
| Sequence 392 | U63810 | GENBANK |
| Sequence 393 | U65928 | GENBANK |
| Sequence 394 | U66616 | GENBANK |
| Sequence 395 | U70735 | GENBANK |
| Sequence 396 | U80034 | GENBANK |
| Sequence 397 | U80743 | GENBANK |
| Sequence 398 | U81001 | GENBANK |
| Sequence 399 | U90028 | GENBANK |
| Sequence 400 | U90144 | GENBANK |
| Sequence 401 | U90916 | GENBANK |
| Sequence 402 | V00478 | GENBANK |
| Sequence 403 | X01742 | GENBANK |
| Sequence 404 | X02422 | GENBANK |
| Sequence 405 | X02457 | GENBANK |
| Sequence 406 | X02544 | GENBANK |
| Sequence 407 | X04098 | GENBANK |
| Sequence 408 | X04412 | GENBANK |
| Sequence 409 | X04470 | GENBANK |
| Sequence 410 | X04503 | GENBANK |
| Sequence 411 | X05276 | GENBANK |
| Sequence 412 | X05332 | GENBANK |
| Sequence 413 | X05908 | GENBANK |
| Sequence 414 | X07695 | GENBANK |
| Sequence 415 | X07820 | GENBANK |
| Sequence 416 | X12597 | GENBANK |
| Sequence 417 | X13425 | GENBANK |
| Sequence 418 | X14420 | GENBANK |
| Sequence 419 | X15187 | GENBANK |
| Sequence 420 | X15729 | GENBANK |
| Sequence 421 | X15822 | GENBANK |
| Sequence 422 | X15880 | GENBANK |
| Sequence 423 | X16940 | GENBANK |
| Sequence 424 | X52022 | GENBANK |
| Sequence 425 | X53280 | GENBANK |
| Sequence 426 | X54941 | GENBANK |
| Sequence 427 | X55525 | GENBANK |
| Sequence 428 | X57810 | GENBANK |
| Sequence 429 | X59618 | GENBANK |
| Sequence 430 | X61970 | GENBANK |
| Sequence 431 | X62744 | GENBANK |
| Sequence 432 | X63432 | GENBANK |
| Sequence 433 | X64875 | GENBANK |
| Sequence 434 | X65550 | GENBANK |
| Sequence 435 | X66276 | GENBANK |
| Sequence 436 | X70326 | GENBANK |
| Sequence 437 | X71087 | GENBANK |
| Sequence 438 | X73114 | GENBANK |
| Sequence 439 | X73902 | GENBANK |
| Sequence 440 | X74801 | GENBANK |
| Sequence 441 | X75593 | GENBANK |
| Sequence 442 | X75861 | GENBANK |
| Sequence 443 | X76180 | GENBANK |
| Sequence 444 | X78926 | GENBANK |
| Sequence 445 | X85372 | GENBANK |
| Sequence 446 | X87241 | GENBANK |
| Sequence 447 | X90568 | GENBANK |
| Sequence 448 | X90870 | GENBANK |
| Sequence 449 | X91257 | GENBANK |
| Sequence 450 | X94323 | GENBANK |
| Sequence 451 | X94754 | GENBANK |
| Sequence 452 | X95240 | GENBANK |
| Sequence 453 | X95677 | GENBANK |
| Sequence 454 | X99920 | GENBANK |
| Sequence 455 | Y00052 | GENBANK |
| Sequence 456 | Y00281 | GENBANK |
| Sequence 457 | Y00361 | GENBANK |
| Sequence 458 | Y00711 | GENBANK |
| Sequence 459 | Y08915 | GENBANK |
| Sequence 460 | Y09267 | GENBANK |
| Sequence 461 | Y16241 | GENBANK |
| Sequence 462 | Z11338 | GENBANK |
| Sequence 463 | Z18538 | GENBANK |
| Sequence 464 | Z23064 | GENBANK |
| Sequence 465 | Z26317 | GENBANK |
| Sequence 466 | Z29330 | GENBANK |
| Sequence 467 | Z36785 | GENBANK |
| Sequence 468 | Z74615 | GENBANK |
| Sequence 469 | AA005035 | dbEST |
| Sequence 470 | AA007280 | dbEST |
| Sequence 471 | AA010019 | dbEST |
| Sequence 472 | AA010083 | dbEST |
| Sequence 473 | AA012927 | dbEST |
| Sequence 474 | AA016274 | dbEST |
| Sequence 475 | AA018892 | dbEST |
| Sequence 476 | AA019498 | dbEST |
| Sequence 477 | AA029415 | dbEST |
| Sequence 478 | AA031483 | dbEST |
| Sequence 479 | AA031762 | dbEST |
| Sequence 480 | AA033869 | dbEST |
| Sequence 481 | AA034226 | dbEST |
| Sequence 482 | AA034237 | dbEST |
| Sequence 483 | AA035742 | dbEST |
| Sequence 484 | AA037126 | dbEST |
| Sequence 485 | AA037172 | dbEST |
| Sequence 486 | AA037554 | dbEST |
| Sequence 487 | AA039354 | dbEST |
| Sequence 488 | AA039504 | dbEST |
| Sequence 489 | AA040209 | dbEST |
| Sequence 490 | AA041494 | dbEST |
| Sequence 491 | AA043431 | dbEST |
| Sequence 492 | AA044187 | dbEST |
| Sequence 493 | AA044209 | dbEST |
| Sequence 494 | AA044416 | dbEST |
| Sequence 495 | AA044691 | dbEST |
| Sequence 496 | AA044960 | dbEST |
| Sequence 497 | AA045584 | dbEST |
| Sequence 498 | AA047213 | dbEST |
| Sequence 499 | AA047332 | dbEST |
| Sequence 500 | AA047704 | dbEST |
| Sequence 501 | AA053522 | dbEST |
| Sequence 502 | AA054521 | dbEST |
| Sequence 503 | AA056281 | dbEST |
| Sequence 504 | AA056686 | dbEST |
| Sequence 505 | AA071030 | dbEST |
| Sequence 506 | AA074067 | dbEST |
| Sequence 507 | AA075008 | dbEST |
| Sequence 508 | AA075515 | dbEST |
| Sequence 509 | AA075645 | dbEST |
| Sequence 510 | AA075981 | dbEST |
| Sequence 511 | AA076171 | dbEST |
| Sequence 512 | AA076964 | dbEST |
| Sequence 513 | AA077283 | dbEST |
| Sequence 514 | AA079706 | dbEST |
| Sequence 515 | AA081788 | dbEST |
| Sequence 516 | AA081800 | dbEST |
| Sequence 517 | AA081973 | dbEST |
| Sequence 518 | AA082437 | dbEST |
| Sequence 519 | AA083654 | dbEST |
| Sequence 520 | AA086007 | dbEST |
| Sequence 521 | AA088322 | dbEST |
| Sequence 522 | AA088850 | dbEST |
| Sequence 523 | AA092224 | dbEST |
| Sequence 524 | AA099952 | dbEST |
| Sequence 525 | AA100987 | dbEST |
| Sequence 526 | AA101208 | dbEST |
| Sequence 527 | AA113403 | dbEST |
| Sequence 528 | AA115604 | dbEST |
| Sequence 529 | AA120818 | dbEST |
| Sequence 530 | AA121471 | dbEST |
| Sequence 531 | AA121923 | dbEST |
| Sequence 532 | AA125809 | dbEST |
| Sequence 533 | AA125989 | dbEST |
| Sequence 534 | AA126128 | dbEST |
| Sequence 535 | AA127674 | dbEST |
| Sequence 536 | AA128306 | dbEST |
| Sequence 537 | AA128391 | dbEST |
| Sequence 538 | AA129501 | dbEST |
| Sequence 539 | AA129909 | dbEST |
| Sequence 540 | AA132798 | dbEST |
| Sequence 541 | AA132956 | dbEST |
| Sequence 542 | AA133382 | dbEST |
| Sequence 543 | AA133503 | dbEST |
| Sequence 544 | AA134032 | dbEST |
| Sequence 545 | AA134778 | dbEST |
| Sequence 546 | AA135049 | dbEST |
| Sequence 547 | AA135104 | dbEST |
| Sequence 548 | AA136794 | dbEST |
| Sequence 549 | AA136830 | dbEST |
| Sequence 550 | AA148002 | dbEST |
| Sequence 551 | AA149624 | dbEST |
| Sequence 552 | AA150928 | dbEST |
| Sequence 553 | AA156040 | dbEST |
| Sequence 554 | AA159847 | dbEST |
| Sequence 555 | AA160002 | dbEST |
| Sequence 556 | AA161003 | dbEST |
| Sequence 557 | AA164473 | dbEST |
| Sequence 558 | AA165243 | dbEST |
| Sequence 559 | AA165632 | dbEST |
| Sequence 560 | AA165639 | dbEST |
| Sequence 561 | AA166907 | dbEST |
| Sequence 562 | AA169276 | dbEST |
| Sequence 563 | AA169638 | dbEST |
| Sequence 564 | AA171516 | dbEST |
| Sequence 565 | AA171872 | dbEST |
| Sequence 566 | AA179105 | dbEST |
| Sequence 567 | AA180270 | dbEST |
| Sequence 568 | AA181526 | dbEST |
| Sequence 569 | AA181580 | dbEST |
| Sequence 570 | AA182409 | dbEST |
| Sequence 571 | AA186538 | dbEST |
| Sequence 572 | AA186733 | dbEST |
| Sequence 573 | AA187659 | dbEST |
| Sequence 574 | AA187817 | dbEST |
| Sequence 575 | AA188918 | dbEST |
| Sequence 576 | AA190676 | dbEST |
| Sequence 577 | AA194988 | dbEST |
| Sequence 578 | AA195560 | dbEST |
| Sequence 579 | AA196978 | dbEST |
| Sequence 580 | AA203237 | dbEST |
| Sequence 581 | AA205870 | dbEST |
| Sequence 582 | AA211498 | dbEST |
| Sequence 583 | AA213548 | dbEST |
| Sequence 584 | AA215800 | dbEST |
| Sequence 585 | AA218858 | dbEST |
| Sequence 586 | AA223425 | dbEST |
| Sequence 587 | AA224230 | dbEST |
| Sequence 588 | AA225025 | dbEST |
| Sequence 589 | AA229993 | dbEST |
| Sequence 590 | AA232738 | dbEST |
| Sequence 591 | AA233867 | dbEST |
| Sequence 592 | AA234149 | dbEST |
| Sequence 593 | AA234464 | dbEST |
| Sequence 594 | AA234646 | dbEST |
| Sequence 595 | AA234667 | dbEST |
| Sequence 596 | AA247638 | dbEST |
| Sequence 597 | AA250846 | dbEST |
| Sequence 598 | AA250854 | dbEST |
| Sequence 599 | AA253248 | dbEST |
| Sequence 600 | AA255577 | dbEST |
| Sequence 601 | AA256290 | dbEST |
| Sequence 602 | AA256297 | dbEST |
| Sequence 603 | AA256591 | dbEST |
| Sequence 604 | AA258003 | dbEST |
| Sequence 605 | AA261968 | dbEST |
| Sequence 606 | AA262683 | dbEST |
| Sequence 607 | AA262783 | dbEST |
| Sequence 608 | AA278357 | dbEST |
| Sequence 609 | AA278785 | dbEST |
| Sequence 610 | AA278956 | dbEST |
| Sequence 611 | AA279145 | dbEST |
| Sequence 612 | AA279262 | dbEST |
| Sequence 613 | AA282956 | dbEST |
| Sequence 614 | AA283112 | dbEST |
| Sequence 615 | AA285043 | dbEST |
| Sequence 616 | AA291436 | dbEST |
| Sequence 617 | AA292132 | dbEST |
| Sequence 618 | AA292281 | dbEST |
| Sequence 619 | AA293027 | dbEST |
| Sequence 620 | AA295182 | dbEST |
| Sequence 621 | AA295996 | dbEST |
| Sequence 622 | AA297432 | dbEST |
| Sequence 623 | AA298085 | dbEST |
| Sequence 624 | AA298773 | dbEST |
| Sequence 625 | AA298794 | dbEST |
| Sequence 626 | AA300440 | dbEST |
| Sequence 627 | AA303257 | dbEST |
| Sequence 628 | AA304979 | dbEST |
| Sequence 629 | AA305319 | dbEST |
| Sequence 630 | AA305494 | dbEST |
| Sequence 631 | AA306772 | dbEST |
| Sequence 632 | AA307286 | dbEST |
| Sequence 633 | AA307539 | dbEST |
| Sequence 634 | AA307857 | dbEST |
| Sequence 635 | AA309002 | dbEST |
| Sequence 636 | AA309127 | dbEST |
| Sequence 637 | AA310469 | dbEST |
| Sequence 638 | AA311028 | dbEST |
| Sequence 639 | AA312078 | dbEST |
| Sequence 640 | AA312962 | dbEST |
| Sequence 641 | AA313164 | dbEST |
| Sequence 642 | AA313653 | dbEST |
| Sequence 643 | AA313692 | dbEST |
| Sequence 644 | AA313922 | dbEST |
| Sequence 645 | AA315049 | dbEST |
| Sequence 646 | AA316207 | dbEST |
| Sequence 647 | AA318284 | dbEST |
| Sequence 648 | AA319601 | dbEST |
| Sequence 649 | AA319870 | dbEST |
| Sequence 650 | AA329658 | dbEST |
| Sequence 651 | AA334754 | dbEST |
| Sequence 652 | AA340635 | dbEST |
| Sequence 653 | AA342701 | dbEST |
| Sequence 654 | AA345906 | dbEST |
| Sequence 655 | AA348669 | dbEST |
| Sequence 656 | AA352926 | dbEST |
| Sequence 657 | AA355890 | dbEST |
| Sequence 658 | AA357574 | dbEST |
| Sequence 659 | AA360977 | dbEST |
| Sequence 660 | AA361225 | dbEST |
| Sequence 661 | AA363049 | dbEST |
| Sequence 662 | AA371483 | dbEST |
| Sequence 663 | AA376277 | dbEST |
| Sequence 664 | AA376668 | dbEST |
| Sequence 665 | AA386326 | dbEST |
| Sequence 666 | AA398214 | dbEST |
| Sequence 667 | AA398463 | dbEST |
| Sequence 668 | AA398674 | dbEST |
| Sequence 669 | AA399177 | dbEST |
| Sequence 670 | AA399265 | dbEST |
| Sequence 671 | AA400279 | dbEST |
| Sequence 672 | AA401208 | dbEST |
| Sequence 673 | AA401285 | dbEST |
| Sequence 674 | AA410580 | dbEST |
| Sequence 675 | AA411736 | dbEST |
| Sequence 676 | AA417324 | dbEST |
| Sequence 677 | AA418061 | dbEST |
| Sequence 678 | AA418080 | dbEST |
| Sequence 679 | AA418681 | dbEST |
| Sequence 680 | AA419263 | dbEST |
| Sequence 681 | AA421682 | dbEST |
| Sequence 682 | AA423943 | dbEST |
| Sequence 683 | AA425218 | dbEST |
| Sequence 684 | AA425619 | dbEST |
| Sequence 685 | AA428329 | dbEST |
| Sequence 686 | AA429721 | dbEST |
| Sequence 687 | AA430381 | dbEST |
| Sequence 688 | AA430400 | dbEST |
| Sequence 689 | AA431226 | dbEST |
| Sequence 690 | AA432075 | dbEST |
| Sequence 691 | AA435887 | dbEST |
| Sequence 692 | AA436765 | dbEST |
| Sequence 693 | AA443154 | dbEST |
| Sequence 694 | AA443806 | dbEST |
| Sequence 695 | AA443870 | dbEST |
| Sequence 696 | AA447116 | dbEST |
| Sequence 697 | AA448195 | dbEST |
| Sequence 698 | AA449632 | dbEST |
| Sequence 699 | AA449922 | dbEST |
| Sequence 700 | AA453486 | dbEST |
| Sequence 701 | AA453555 | dbEST |
| Sequence 702 | AA456419 | dbEST |
| Sequence 703 | AA456737 | dbEST |
| Sequence 704 | AA458921 | dbEST |
| Sequence 705 | AA459208 | dbEST |
| Sequence 706 | AA459210 | dbEST |
| Sequence 707 | AA461307 | dbEST |
| Sequence 708 | AA464952 | dbEST |
| Sequence 709 | AA465450 | dbEST |
| Sequence 710 | AA468335 | dbEST |
| Sequence 711 | AA468839 | dbEST |
| Sequence 712 | AA476392 | dbEST |
| Sequence 713 | AA477288 | dbEST |
| Sequence 714 | AA477856 | dbEST |
| Sequence 715 | AA478258 | dbEST |
| Sequence 716 | AA478842 | dbEST |
| Sequence 717 | AA481047 | dbEST |
| Sequence 718 | AA482430 | dbEST |
| Sequence 719 | AA482434 | dbEST |
| Sequence 720 | AA483635 | dbEST |
| Sequence 721 | AA488468 | dbEST |
| Sequence 722 | AA488892 | dbEST |
| Sequence 723 | AA489012 | dbEST |
| Sequence 724 | AA489032 | dbEST |
| Sequence 725 | AA490635 | dbEST |
| Sequence 726 | AA492272 | dbEST |
| Sequence 727 | AA492280 | dbEST |
| Sequence 728 | AA492392 | dbEST |
| Sequence 729 | AA493512 | dbEST |
| Sequence 730 | AA502979 | dbEST |
| Sequence 731 | AA503330 | dbEST |
| Sequence 732 | AA505400 | dbEST |
| Sequence 733 | AA507314 | dbEST |
| Sequence 734 | AA508804 | dbEST |
| Sequence 735 | AA514933 | dbEST |
| Sequence 736 | AA522686 | dbEST |
| Sequence 737 | AA523252 | dbEST |
| Sequence 738 | AA523677 | dbEST |
| Sequence 739 | AA523708 | dbEST |
| Sequence 740 | AA524023 | dbEST |
| Sequence 741 | AA524462 | dbEST |
| Sequence 742 | AA526151 | dbEST |
| Sequence 743 | AA527570 | dbEST |
| Sequence 744 | AA527805 | dbEST |
| Sequence 745 | AA531044 | dbEST |
| Sequence 746 | AA531255 | dbEST |
| Sequence 747 | AA531563 | dbEST |
| Sequence 748 | AA532377 | dbEST |
| Sequence 749 | AA533575 | dbEST |
| Sequence 750 | AA534873 | dbEST |
| Sequence 751 | AA541537 | dbEST |
| Sequence 752 | AA541677 | dbEST |
| Sequence 753 | AA548098 | dbEST |
| Sequence 754 | AA548244 | dbEST |
| Sequence 755 | AA548600 | dbEST |
| Sequence 756 | AA551005 | dbEST |
| Sequence 757 | AA553789 | dbEST |
| Sequence 758 | AA554010 | dbEST |
| Sequence 759 | AA554598 | dbEST |
| Sequence 760 | AA554718 | dbEST |
| Sequence 761 | AA555227 | dbEST |
| Sequence 762 | AA558280 | dbEST |
| Sequence 763 | AA564843 | dbEST |
| Sequence 764 | AA565334 | dbEST |
| Sequence 765 | AA568406 | dbEST |
| Sequence 766 | AA572950 | dbEST |
| Sequence 767 | AA573742 | dbEST |
| Sequence 768 | AA574397 | dbEST |
| Sequence 769 | AA576425 | dbEST |
| Sequence 770 | AA577326 | dbEST |
| Sequence 771 | AA577557 | dbEST |
| Sequence 772 | AA578773 | dbEST |
| Sequence 773 | AA579835 | dbEST |
| Sequence 774 | AA581829 | dbEST |
| Sequence 775 | AA583431 | dbEST |
| Sequence 776 | AA583567 | dbEST |
| Sequence 777 | AA583773 | dbEST |
| Sequence 778 | AA583899 | dbEST |
| Sequence 779 | AA583997 | dbEST |
| Sequence 780 | AA586969 | dbEST |
| Sequence 781 | AA587630 | dbEST |
| Sequence 782 | AA594282 | dbEST |
| Sequence 783 | AA594780 | dbEST |
| Sequence 784 | AA594950 | dbEST |
| Sequence 785 | AA599424 | dbEST |
| Sequence 786 | AA599533 | dbEST |
| Sequence 787 | AA602361 | dbEST |
| Sequence 788 | AA602957 | dbEST |
| Sequence 789 | AA604229 | dbEST |
| Sequence 790 | AA610352 | dbEST |
| Sequence 791 | AA617722 | dbEST |
| Sequence 792 | AA620750 | dbEST |
| Sequence 793 | AA622344 | dbEST |
| Sequence 794 | AA628194 | dbEST |
| Sequence 795 | AA629771 | dbEST |
| Sequence 796 | AA630286 | dbEST |
| Sequence 797 | AA631176 | dbEST |
| Sequence 798 | AA631178 | dbEST |
| Sequence 799 | AA631206 | dbEST |
| Sequence 800 | AA632363 | dbEST |
| Sequence 801 | AA641092 | dbEST |
| Sequence 802 | AA643720 | dbEST |
| Sequence 803 | AA644237 | dbEST |
| Sequence 804 | AA649627 | dbEST |
| Sequence 805 | AA653508 | dbEST |
| Sequence 806 | AA654995 | dbEST |
| Sequence 807 | AA657463 | dbEST |
| Sequence 808 | AA662702 | dbEST |
| Sequence 809 | AA665901 | dbEST |
| Sequence 810 | AA669976 | dbEST |
| Sequence 811 | AA683314 | dbEST |
| Sequence 812 | AA687495 | dbEST |
| Sequence 813 | AA702422 | dbEST |
| Sequence 814 | AA704005 | dbEST |
| Sequence 815 | AA705061 | dbEST |
| Sequence 816 | AA713580 | dbEST |
| Sequence 817 | AA722823 | dbEST |
| Sequence 818 | AA729119 | dbEST |
| Sequence 819 | AA740151 | dbEST |
| Sequence 820 | AA744239 | dbEST |
| Sequence 821 | AA745059 | dbEST |
| Sequence 822 | AA745616 | dbEST |
| Sequence 823 | AA746353 | dbEST |
| Sequence 824 | AA748038 | dbEST |
| Sequence 825 | AA748444 | dbEST |
| Sequence 826 | AA748525 | dbEST |
| Sequence 827 | AA758592 | dbEST |
| Sequence 828 | AA767439 | dbEST |
| Sequence 829 | AA769127 | dbEST |
| Sequence 830 | AA769937 | dbEST |
| Sequence 831 | AA774523 | dbEST |
| Sequence 832 | AA774750 | dbEST |
| Sequence 833 | AA777489 | dbEST |
| Sequence 834 | AA779728 | dbEST |
| Sequence 835 | AA779949 | dbEST |
| Sequence 836 | AA782592 | dbEST |
| Sequence 837 | AA806040 | dbEST |
| Sequence 838 | AA814414 | dbEST |
| Sequence 839 | AA826906 | dbEST |
| Sequence 840 | AA827331 | dbEST |
| Sequence 841 | AA834102 | dbEST |
| Sequence 842 | AA834946 | dbEST |
| Sequence 843 | AA845374 | dbEST |
| Sequence 844 | AA847210 | dbEST |
| Sequence 845 | AA854319 | dbEST |
| Sequence 846 | AA858389 | dbEST |
| Sequence 847 | AA858396 | dbEST |
| Sequence 848 | AA860287 | dbEST |
| Sequence 849 | AA861428 | dbEST |
| Sequence 850 | AA861631 | dbEST |
| Sequence 851 | AA872729 | dbEST |
| Sequence 852 | AA873110 | dbEST |
| Sequence 853 | AA877864 | dbEST |
| Sequence 854 | AA878781 | dbEST |
| Sequence 855 | AA883580 | dbEST |
| Sequence 856 | AA884922 | dbEST |
| Sequence 857 | AA888121 | dbEST |
| Sequence 858 | AA888589 | dbEST |
| Sequence 859 | AA890660 | dbEST |
| Sequence 860 | AA906652 | dbEST |
| Sequence 861 | AA910369 | dbEST |
| Sequence 862 | AA911154 | dbEST |
| Sequence 863 | AA911317 | dbEST |
| Sequence 864 | AA918023 | dbEST |
| Sequence 865 | AA918993 | dbEST |
| Sequence 866 | AA923172 | dbEST |
| Sequence 867 | AA926926 | dbEST |
| Sequence 868 | AA928420 | dbEST |
| Sequence 869 | AA934879 | dbEST |
| Sequence 870 | AA935798 | dbEST |
| Sequence 871 | AA936187 | dbEST |
| Sequence 872 | AA946995 | dbEST |
| Sequence 873 | AA953328 | dbEST |
| Sequence 874 | AA953391 | dbEST |
| Sequence 875 | AA954939 | dbEST |
| Sequence 876 | AA962550 | dbEST |
| Sequence 877 | AA968756 | dbEST |
| Sequence 878 | AA971700 | dbEST |
| Sequence 879 | AA972625 | dbEST |
| Sequence 880 | AA973273 | dbEST |
| Sequence 881 | AA977226 | dbEST |
| Sequence 882 | AA984043 | dbEST |
| Sequence 883 | AA984690 | dbEST |
| Sequence 884 | AA988468 | dbEST |
| Sequence 885 | AA992583 | dbEST |
| Sequence 886 | AA992596 | dbEST |
| Sequence 887 | AA993550 | dbEST |
| Sequence 888 | AF001542 | dbEST |
| Sequence 889 | AF010244 | dbEST |
| Sequence 890 | AF156972 | dbEST |
| Sequence 891 | AI003778 | dbEST |
| Sequence 892 | AI016113 | dbEST |
| Sequence 893 | AI016417 | dbEST |
| Sequence 894 | AI017236 | dbEST |
| Sequence 895 | AI018468 | dbEST |
| Sequence 896 | AI018578 | dbEST |
| Sequence 897 | AI021925 | dbEST |
| Sequence 898 | AI022353 | dbEST |
| Sequence 899 | AI026819 | dbEST |
| Sequence 900 | AI027232 | dbEST |
| Sequence 901 | AI031819 | dbEST |
| Sequence 902 | AI033904 | dbEST |
| Sequence 903 | AI038986 | dbEST |
| Sequence 904 | AI041820 | dbEST |
| Sequence 905 | AI042290 | dbEST |
| Sequence 906 | AI051987 | dbEST |
| Sequence 907 | AI052124 | dbEST |
| Sequence 908 | AI052525 | dbEST |
| Sequence 909 | AI064691 | dbEST |
| Sequence 910 | AI074713 | dbEST |
| Sequence 911 | AI075189 | dbEST |
| Sequence 912 | AI075211 | dbEST |
| Sequence 913 | AI079329 | dbEST |
| Sequence 914 | AI080377 | dbEST |
| Sequence 915 | AI082168 | dbEST |
| Sequence 916 | AI084457 | dbEST |
| Sequence 917 | AI084466 | dbEST |
| Sequence 918 | AI089525 | dbEST |
| Sequence 919 | AI090623 | dbEST |
| Sequence 920 | AI091425 | dbEST |
| Sequence 921 | AI092428 | dbEST |
| Sequence 922 | AI092766 | dbEST |
| Sequence 923 | AI093553 | dbEST |
| Sequence 924 | AI094010 | dbEST |
| Sequence 925 | AI094162 | dbEST |
| Sequence 926 | AI110856 | dbEST |
| Sequence 927 | AI110866 | dbEST |
| Sequence 928 | AI114467 | dbEST |
| Sequence 929 | AI126093 | dbEST |
| Sequence 930 | AI127239 | dbEST |
| Sequence 931 | AI129932 | dbEST |
| Sequence 932 | AI133138 | dbEST |
| Sequence 933 | AI133208 | dbEST |
| Sequence 934 | AI133387 | dbEST |
| Sequence 935 | AI141847 | dbEST |
| Sequence 936 | AI143199 | dbEST |
| Sequence 937 | AI143899 | dbEST |
| Sequence 938 | AI148251 | dbEST |
| Sequence 939 | AI151497 | dbEST |
| Sequence 940 | AI161261 | dbEST |
| Sequence 941 | AI161272 | dbEST |
| Sequence 942 | AI167330 | dbEST |
| Sequence 943 | AI174394 | dbEST |
| Sequence 944 | AI189173 | dbEST |
| Sequence 945 | AI189340 | dbEST |
| Sequence 946 | AI189841 | dbEST |
| Sequence 947 | AI198317 | dbEST |
| Sequence 948 | AI198416 | dbEST |
| Sequence 949 | AI200830 | dbEST |
| Sequence 950 | AI207570 | dbEST |
| Sequence 951 | AI207628 | dbEST |
| Sequence 952 | AI214054 | dbEST |
| Sequence 953 | AI215841 | dbEST |
| Sequence 954 | AI216971 | dbEST |
| Sequence 955 | AI216979 | dbEST |
| Sequence 956 | AI216988 | dbEST |
| Sequence 957 | AI217003 | dbEST |
| Sequence 958 | AI217019 | dbEST |
| Sequence 959 | AI219116 | dbEST |
| Sequence 960 | AI220151 | dbEST |
| Sequence 961 | AI221894 | dbEST |
| Sequence 962 | AI223085 | dbEST |
| Sequence 963 | AI241212 | dbEST |
| Sequence 964 | AI242761 | dbEST |
| Sequence 965 | AI243343 | dbEST |
| Sequence 966 | AI249866 | dbEST |
| Sequence 967 | AI253330 | dbEST |
| Sequence 968 | AI253381 | dbEST |
| Sequence 969 | AI253436 | dbEST |
| Sequence 970 | AI261899 | dbEST |
| Sequence 971 | AI267162 | dbEST |
| Sequence 972 | AI267185 | dbEST |
| Sequence 973 | AI267282 | dbEST |
| Sequence 974 | AI267335 | dbEST |
| Sequence 975 | AI267417 | dbEST |
| Sequence 976 | AI267442 | dbEST |
| Sequence 977 | AI267454 | dbEST |
| Sequence 978 | AI267502 | dbEST |
| Sequence 979 | AI268430 | dbEST |
| Sequence 980 | AI269060 | dbEST |
| Sequence 981 | AI271740 | dbEST |
| Sequence 982 | AI272941 | dbEST |
| Sequence 983 | AI276322 | dbEST |
| Sequence 984 | AI276341 | dbEST |
| Sequence 985 | AI278838 | dbEST |
| Sequence 986 | AI280637 | dbEST |
| Sequence 987 | AI281509 | dbEST |
| Sequence 988 | AI281762 | dbEST |
| Sequence 989 | AI291206 | dbEST |
| Sequence 990 | AI298716 | dbEST |
| Sequence 991 | AI304947 | dbEST |
| Sequence 992 | AI309238 | dbEST |
| Sequence 993 | AI313352 | dbEST |
| Sequence 994 | AI332459 | dbEST |
| Sequence 995 | AI334996 | dbEST |
| Sequence 996 | AI338817 | dbEST |
| Sequence 997 | AI340582 | dbEST |
| Sequence 998 | AI343367 | dbEST |
| Sequence 999 | AI359849 | dbEST |
| Sequence 1000 | AI361377 | dbEST |
| Sequence 1001 | AI366459 | dbEST |
| Sequence 1002 | AI366549 | dbEST |
| Sequence 1003 | AI368048 | dbEST |
| Sequence 1004 | AI368114 | dbEST |
| Sequence 1005 | AI371832 | dbEST |
| Sequence 1006 | AI374643 | dbEST |
| Sequence 1007 | AI374954 | dbEST |
| Sequence 1008 | AI376808 | dbEST |
| Sequence 1009 | AI378631 | dbEST |
| Sequence 1010 | AI378902 | dbEST |
| Sequence 1011 | AI379402 | dbEST |
| Sequence 1012 | AI381351 | dbEST |
| Sequence 1013 | AI382980 | dbEST |
| Sequence 1014 | AI392607 | dbEST |
| Sequence 1015 | AI399717 | dbEST |
| Sequence 1016 | AI400752 | dbEST |
| Sequence 1017 | AI421761 | dbEST |
| Sequence 1018 | AI422243 | dbEST |
| Sequence 1019 | AI431675 | dbEST |
| Sequence 1020 | AI431869 | dbEST |
| Sequence 1021 | AI432969 | dbEST |
| Sequence 1022 | AI433717 | dbEST |
| Sequence 1023 | AI439248 | dbEST |
| Sequence 1024 | AI439762 | dbEST |
| Sequence 1025 | AI445471 | dbEST |
| Sequence 1026 | AI445592 | dbEST |
| Sequence 1027 | AI458823 | dbEST |
| Sequence 1028 | AI470061 | dbEST |
| Sequence 1029 | AI473310 | dbEST |
| Sequence 1030 | AI474750 | dbEST |
| Sequence 1031 | AI492263 | dbEST |
| Sequence 1032 | AI493961 | dbEST |
| Sequence 1033 | AI524874 | dbEST |
| Sequence 1034 | AI524991 | dbEST |
| Sequence 1035 | AI557112 | dbEST |
| Sequence 1036 | AI557116 | dbEST |
| Sequence 1037 | AI557182 | dbEST |
| Sequence 1038 | AI557225 | dbEST |
| Sequence 1039 | AI557495 | dbEST |
| Sequence 1040 | AI557599 | dbEST |
| Sequence 1041 | AI557626 | dbEST |
| Sequence 1042 | AI559680 | dbEST |
| Sequence 1043 | AI564173 | dbEST |
| Sequence 1044 | AI564269 | dbEST |
| Sequence 1045 | AI569955 | dbEST |
| Sequence 1046 | AI571517 | dbEST |
| Sequence 1047 | AI571730 | dbEST |
| Sequence 1048 | AI573140 | dbEST |
| Sequence 1049 | AI573212 | dbEST |
| Sequence 1050 | AI582365 | dbEST |
| Sequence 1051 | AI587300 | dbEST |
| Sequence 1052 | AI588866 | dbEST |
| Sequence 1053 | AI589285 | dbEST |
| Sequence 1054 | AI589756 | dbEST |
| Sequence 1055 | AI613093 | dbEST |
| Sequence 1056 | AI623176 | dbEST |
| Sequence 1057 | AI623804 | dbEST |
| Sequence 1058 | AI624249 | dbEST |
| Sequence 1059 | AI624441 | dbEST |
| Sequence 1060 | AI628885 | dbEST |
| Sequence 1061 | AI631745 | dbEST |
| Sequence 1062 | AI634702 | dbEST |
| Sequence 1063 | AI635096 | dbEST |
| Sequence 1064 | AI654284 | dbEST |
| Sequence 1065 | AI654454 | dbEST |
| Sequence 1066 | AI655189 | dbEST |
| Sequence 1067 | AI656323 | dbEST |
| Sequence 1068 | AI658689 | dbEST |
| Sequence 1069 | AI680218 | dbEST |
| Sequence 1070 | AI680242 | dbEST |
| Sequence 1071 | AI682287 | dbEST |
| Sequence 1072 | AI683094 | dbEST |
| Sequence 1073 | AI683431 | dbEST |
| Sequence 1074 | AI684157 | dbEST |
| Sequence 1075 | AI688187 | dbEST |
| Sequence 1076 | AI689722 | dbEST |
| Sequence 1077 | AI693493 | dbEST |
| Sequence 1078 | AI697849 | dbEST |
| Sequence 1079 | AI698198 | dbEST |
| Sequence 1080 | AI703405 | dbEST |
| Sequence 1081 | AI720403 | dbEST |
| Sequence 1082 | AI733862 | dbEST |
| Sequence 1083 | AI740534 | dbEST |
| Sequence 1084 | AI740792 | dbEST |
| Sequence 1085 | AI740796 | dbEST |
| Sequence 1086 | AI740900 | dbEST |
| Sequence 1087 | AI743852 | dbEST |
| Sequence 1088 | AI750535 | dbEST |
| Sequence 1089 | AI750567 | dbEST |
| Sequence 1090 | AI750846 | dbEST |
| Sequence 1091 | AI751565 | dbEST |
| Sequence 1092 | AI753269 | dbEST |
| Sequence 1093 | AI754437 | dbEST |
| Sequence 1094 | AI755085 | dbEST |
| Sequence 1095 | AI763239 | dbEST |
| Sequence 1096 | AI768132 | dbEST |
| Sequence 1097 | AI792191 | dbEST |
| Sequence 1098 | AI792758 | dbEST |
| Sequence 1099 | AI799502 | dbEST |
| Sequence 1100 | AI800896 | dbEST |
| Sequence 1101 | AI804346 | dbEST |
| Sequence 1102 | AI804983 | dbEST |
| Sequence 1103 | AI810626 | dbEST |
| Sequence 1104 | AI811498 | dbEST |
| Sequence 1105 | AI814148 | dbEST |
| Sequence 1106 | AI815829 | dbEST |
| Sequence 1107 | AI819225 | dbEST |
| Sequence 1108 | AI819564 | dbEST |
| Sequence 1109 | AI819701 | dbEST |
| Sequence 1110 | AI859619 | dbEST |
| Sequence 1111 | AI869280 | dbEST |
| Sequence 1112 | AI879992 | dbEST |
| Sequence 1113 | AI890387 | dbEST |
| Sequence 1114 | AI890616 | dbEST |
| Sequence 1115 | AI911059 | dbEST |
| Sequence 1116 | AI914030 | dbEST |
| Sequence 1117 | AI914128 | dbEST |
| Sequence 1118 | AI916584 | dbEST |
| Sequence 1119 | AI922760 | dbEST |
| Sequence 1120 | AI923574 | dbEST |
| Sequence 1121 | AI927301 | dbEST |
| Sequence 1122 | AI936548 | dbEST |
| Sequence 1123 | AI952294 | dbEST |
| Sequence 1124 | AI955769 | dbEST |
| Sequence 1125 | AI984541 | dbEST |
| Sequence 1126 | AI984763 | dbEST |
| Sequence 1127 | AJ243229 | dbEST |
| Sequence 1128 | AL035998 | dbEST |
| Sequence 1129 | AL036354 | dbEST |
| Sequence 1130 | AL037724 | dbEST |
| Sequence 1131 | AL039293 | dbEST |
| Sequence 1132 | AL040084 | dbEST |
| Sequence 1133 | AL040993 | dbEST |
| Sequence 1134 | AL042815 | dbEST |
| Sequence 1135 | AL043346 | dbEST |
| Sequence 1136 | AL045297 | dbEST |
| Sequence 1137 | AL110357 | dbEST |
| Sequence 1138 | AL119728 | dbEST |
| Sequence 1139 | AW003247 | dbEST |
| Sequence 1140 | AW003967 | dbEST |
| Sequence 1141 | AW009562 | dbEST |
| Sequence 1142 | AW015055 | dbEST |
| Sequence 1143 | AW025074 | dbEST |
| Sequence 1144 | AW027250 | dbEST |
| Sequence 1145 | AW027586 | dbEST |
| Sequence 1146 | AW044029 | dbEST |
| Sequence 1147 | AW044114 | dbEST |
| Sequence 1148 | AW068491 | dbEST |
| Sequence 1149 | AW090544 | dbEST |
| Sequence 1150 | AW104641 | dbEST |
| Sequence 1151 | AW118812 | dbEST |
| Sequence 1152 | AW157303 | dbEST |
| Sequence 1153 | AW170193 | dbEST |
| Sequence 1154 | C04376 | dbEST |
| Sequence 1155 | F07019 | dbEST |
| Sequence 1156 | F07647 | dbEST |
| Sequence 1157 | H02308 | dbEST |
| Sequence 1158 | H03785 | dbEST |
| Sequence 1159 | H08511 | dbEST |
| Sequence 1160 | H08542 | dbEST |
| Sequence 1161 | H08959 | dbEST |
| Sequence 1162 | H10107 | dbEST |
| Sequence 1163 | H10555 | dbEST |
| Sequence 1164 | H12229 | dbEST |
| Sequence 1165 | H12519 | dbEST |
| Sequence 1166 | H14985 | dbEST |
| Sequence 1167 | H22734 | dbEST |
| Sequence 1168 | H23157 | dbEST |
| Sequence 1169 | H26678 | dbEST |
| Sequence 1170 | H29664 | dbEST |
| Sequence 1171 | H38914 | dbEST |
| Sequence 1172 | H54680 | dbEST |
| Sequence 1173 | H57585 | dbEST |
| Sequence 1174 | H57648 | dbEST |
| Sequence 1175 | H61886 | dbEST |
| Sequence 1176 | H64719 | dbEST |
| Sequence 1177 | H73335 | dbEST |
| Sequence 1178 | H83784 | dbEST |
| Sequence 1179 | H84729 | dbEST |
| Sequence 1180 | H92106 | dbEST |
| Sequence 1181 | H94927 | dbEST |
| Sequence 1182 | H99611 | dbEST |
| Sequence 1183 | M62015 | dbEST |
| Sequence 1184 | N25751 | dbEST |
| Sequence 1185 | N28308 | dbEST |
| Sequence 1186 | N36968 | dbEST |
| Sequence 1187 | N42196 | dbEST |
| Sequence 1188 | N42772 | dbEST |
| Sequence 1189 | N63991 | dbEST |
| Sequence 1190 | N77277 | dbEST |
| Sequence 1191 | N78279 | dbEST |
| Sequence 1192 | N81205 | dbEST |
| Sequence 1193 | N91096 | dbEST |
| Sequence 1194 | N94855 | dbEST |
| Sequence 1195 | N99553 | dbEST |
| Sequence 1196 | N99698 | dbEST |
| Sequence 1197 | R13761 | dbEST |
| Sequence 1198 | R33779 | dbEST |
| Sequence 1199 | R48977 | dbEST |
| Sequence 1200 | R50104 | dbEST |
| Sequence 1201 | R61734 | dbEST |
| Sequence 1202 | R64693 | dbEST |
| Sequence 1203 | R67466 | dbEST |
| Sequence 1204 | R70639 | dbEST |
| Sequence 1205 | R77186 | dbEST |
| Sequence 1206 | R82429 | dbEST |
| Sequence 1207 | R96147 | dbEST |
| Sequence 1208 | R99043 | dbEST |
| Sequence 1209 | T78419 | dbEST |
| Sequence 1210 | T88702 | dbEST |
| Sequence 1211 | W05463 | dbEST |
| Sequence 1212 | W15477 | dbEST |
| Sequence 1213 | W56388 | dbEST |
| Sequence 1214 | W87522 | dbEST |
| Sequence 1215 | W89057 | dbEST |
| Sequence 1216 | Z25142 | dbEST |
| Sequence 1217 | Z36731 | dbEST |
| Sequence 1218 | Q37741 | NUCPATENT |
| Sequence 1219 | T19093 | NUCPATENT |
| Sequence 1220 | T59274 | NUCPATENT |
| Sequence 1221 | V43261 | NUCPATENT |
| Sequence 1222 | V43613 | NUCPATENT |
| Sequence 1223 | V59617 | NUCPATENT |
| Sequence 1224 | V62427 | NUCPATENT |
| Sequence 1225 | V62428 | NUCPATENT |
| Sequence 1226 | V63637 | NUCPATENT |
| Sequence 1227 | V74172 | NUCPATENT |
| Sequence 1228 | V84428 | NUCPATENT |
| Sequence 1229 | X02974 | NUCPATENT |
| Sequence 1230 | X03841 | NUCPATENT |
| Sequence 1231 | X04408 | NUCPATENT |
| Sequence 1232 | X22111 | NUCPATENT |
| Sequence 1233 | X27262 | NUCPATENT |
| Sequence 1234 | X27329 | NUCPATENT |
| Sequence 1235 | X30167 | NUCPATENT |
| Sequence 1236 | X37486 | NUCPATENT |
| Sequence 1237 | X97677 | NUCPATENT |
| Sequence 1238 | Z18356 | NUCPATENT |
| Sequence 1239 | Z24611 | NUCPATENT |
| Sequence 1240 | AC31093 | PREPATNUC |
| Sequence 1241 | A18657 | GENBANK |
| Sequence 1242 | AB000095 | GENBANK |
| Sequence 1243 | AB000220 | GENBANK |
| Sequence 1244 | AB002366 | GENBANK |
| Sequence 1245 | AB002387 | GENBANK |
| Sequence 1246 | AB002391 | GENBANK |
| Sequence 1247 | AB007191 | GENBANK |
| Sequence 1248 | AB007883 | GENBANK |
| Sequence 1249 | AB008109 | GENBANK |
| Sequence 1250 | AB009285 | GENBANK |
| Sequence 1251 | AB010882 | GENBANK |
| Sequence 1252 | AB011175 | GENBANK |
| Sequence 1253 | AB013897 | GENBANK |
| Sequence 1254 | AB014511 | GENBANK |
| Sequence 1255 | AB014514 | GENBANK |
| Sequence 1256 | AB014525 | GENBANK |
| Sequence 1257 | AB014531 | GENBANK |
| Sequence 1258 | AB014536 | GENBANK |
| Sequence 1259 | AB014563 | GENBANK |
| Sequence 1260 | AB014569 | GENBANK |
| Sequence 1261 | AB014610 | GENBANK |
| Sequence 1262 | AB018257 | GENBANK |
| Sequence 1263 | AB018304 | GENBANK |
| Sequence 1264 | AB018344 | GENBANK |
| Sequence 1265 | AB019002 | GENBANK |
| Sequence 1266 | AB019563 | GENBANK |
| Sequence 1267 | AB019568 | GENBANK |
| Sequence 1268 | AB020637 | GENBANK |
| Sequence 1269 | AB020686 | GENBANK |
| Sequence 1270 | AB020692 | GENBANK |
| Sequence 1271 | AB020707 | GENBANK |
| Sequence 1272 | AB021179 | GENBANK |
| Sequence 1273 | AB021288 | GENBANK |
| Sequence 1274 | AB022663 | GENBANK |
| Sequence 1275 | AB023145 | GENBANK |
| Sequence 1276 | AB028969 | GENBANK |
| Sequence 1277 | AB029036 | GENBANK |
| Sequence 1278 | AB032969 | GENBANK |
| Sequence 1279 | AB032990 | GENBANK |
| Sequence 1280 | AB033029 | GENBANK |
| Sequence 1281 | AB033069 | GENBANK |
| Sequence 1282 | AB033078 | GENBANK |
| Sequence 1283 | AF001893 | GENBANK |
| Sequence 1284 | AF004162 | GENBANK |
| Sequence 1285 | AF004327 | GENBANK |
| Sequence 1286 | AF004813 | GENBANK |
| Sequence 1287 | AF006514 | GENBANK |
| Sequence 1288 | AF007135 | GENBANK |
| Sequence 1289 | AF007216 | GENBANK |
| Sequence 1290 | AF007791 | GENBANK |
| Sequence 1291 | AF008915 | GENBANK |
| Sequence 1292 | AF010233 | GENBANK |
| Sequence 1293 | AF015040 | GENBANK |
| Sequence 1294 | AF016369 | GENBANK |
| Sequence 1295 | AF020038 | GENBANK |
| Sequence 1296 | AF020202 | GENBANK |
| Sequence 1297 | AF020736 | GENBANK |
| Sequence 1298 | AF021819 | GENBANK |
| Sequence 1299 | AF026166 | GENBANK |
| Sequence 1300 | AF026291 | GENBANK |
| Sequence 1301 | AF027824 | GENBANK |
| Sequence 1302 | AF031647 | GENBANK |
| Sequence 1303 | AF032885 | GENBANK |
| Sequence 1304 | AF034759 | GENBANK |
| Sequence 1305 | AF035286 | GENBANK |
| Sequence 1306 | AF035537 | GENBANK |
| Sequence 1307 | AF035839 | GENBANK |
| Sequence 1308 | AF038451 | GENBANK |
| Sequence 1309 | AF038963 | GENBANK |
| Sequence 1310 | AF039918 | GENBANK |
| Sequence 1311 | AF041260 | GENBANK |
| Sequence 1312 | AF042346 | GENBANK |
| Sequence 1313 | AF045167 | GENBANK |
| Sequence 1314 | AF047020 | GENBANK |
| Sequence 1315 | AF047185 | GENBANK |
| Sequence 1316 | AF047440 | GENBANK |
| Sequence 1317 | AF050637 | GENBANK |
| Sequence 1318 | AF050638 | GENBANK |
| Sequence 1319 | AF051941 | GENBANK |
| Sequence 1320 | AF052138 | GENBANK |
| Sequence 1321 | AF052153 | GENBANK |
| Sequence 1322 | AF052159 | GENBANK |
| Sequence 1323 | AF052183 | GENBANK |
| Sequence 1324 | AF052642 | GENBANK |
| Sequence 1325 | AF054284 | GENBANK |
| Sequence 1326 | AF054838 | GENBANK |
| Sequence 1327 | AF061258 | GENBANK |
| Sequence 1328 | AF061737 | GENBANK |
| Sequence 1329 | AF065388 | GENBANK |
| Sequence 1330 | AF065391 | GENBANK |
| Sequence 1331 | AF068180 | GENBANK |
| Sequence 1332 | AF068235 | GENBANK |
| Sequence 1333 | AF068754 | GENBANK |
| Sequence 1334 | AF069762 | GENBANK |
| Sequence 1335 | AF070523 | GENBANK |
| Sequence 1336 | AF070553 | GENBANK |
| Sequence 1337 | AF070561 | GENBANK |
| Sequence 1338 | AF070562 | GENBANK |
| Sequence 1339 | AF070595 | GENBANK |
| Sequence 1340 | AF070626 | GENBANK |
| Sequence 1341 | AF070650 | GENBANK |
| Sequence 1342 | AF070658 | GENBANK |
| Sequence 1343 | AF070668 | GENBANK |
| Sequence 1344 | AF071202 | GENBANK |
| Sequence 1345 | AF073298 | GENBANK |
| Sequence 1346 | AF073475 | GENBANK |
| Sequence 1347 | AF077029 | GENBANK |
| Sequence 1348 | AF077032 | GENBANK |
| Sequence 1349 | AF077045 | GENBANK |
| Sequence 1350 | AF077200 | GENBANK |
| Sequence 1351 | AF077367 | GENBANK |
| Sequence 1352 | AF078845 | GENBANK |
| Sequence 1353 | AF078847 | GENBANK |
| Sequence 1354 | AF078860 | GENBANK |
| Sequence 1355 | AF078862 | GENBANK |
| Sequence 1356 | AF084457 | GENBANK |
| Sequence 1357 | AF085844 | GENBANK |
| Sequence 1358 | AF085845 | GENBANK |
| Sequence 1359 | AF085850 | GENBANK |
| Sequence 1360 | AF086172 | GENBANK |
| Sequence 1361 | AF086336 | GENBANK |
| Sequence 1362 | AF086351 | GENBANK |
| Sequence 1363 | AF086418 | GENBANK |
| Sequence 1364 | AF086495 | GENBANK |
| Sequence 1365 | AF086557 | GENBANK |
| Sequence 1366 | AF090327 | GENBANK |
| Sequence 1367 | AF091071 | GENBANK |
| Sequence 1368 | AF093668 | GENBANK |
| Sequence 1369 | AF095687 | GENBANK |
| Sequence 1370 | AF098951 | GENBANK |
| Sequence 1371 | AF100741 | GENBANK |
| Sequence 1372 | AF100756 | GENBANK |
| Sequence 1373 | AF102265 | GENBANK |
| Sequence 1374 | AF102846 | GENBANK |
| Sequence 1375 | AF103907 | GENBANK |
| Sequence 1376 | AF111713 | GENBANK |
| Sequence 1377 | AF112972 | GENBANK |
| Sequence 1378 | AF113140 | GENBANK |
| Sequence 1379 | AF117754 | GENBANK |
| Sequence 1380 | AF117756 | GENBANK |
| Sequence 1381 | AF118124 | GENBANK |
| Sequence 1382 | AF125097 | GENBANK |
| Sequence 1383 | AF125100 | GENBANK |
| Sequence 1384 | AF125393 | GENBANK |
| Sequence 1385 | AF126245 | GENBANK |
| Sequence 1386 | AF129927 | GENBANK |
| Sequence 1387 | AF131738 | GENBANK |
| Sequence 1388 | AF131792 | GENBANK |
| Sequence 1389 | AF132968 | GENBANK |
| Sequence 1390 | AF138300 | GENBANK |
| Sequence 1391 | AF143886 | GENBANK |
| Sequence 1392 | AF147331 | GENBANK |
| Sequence 1393 | AF151103 | GENBANK |
| Sequence 1394 | AF151820 | GENBANK |
| Sequence 1395 | AF151840 | GENBANK |
| Sequence 1396 | AF151844 | GENBANK |
| Sequence 1397 | AF151896 | GENBANK |
| Sequence 1398 | AF153201 | GENBANK |
| Sequence 1399 | AF159056 | GENBANK |
| Sequence 1400 | AF176574 | GENBANK |
| Sequence 1401 | AF188745 | GENBANK |
| Sequence 1402 | AF191018 | GENBANK |
| Sequence 1403 | AF191020 | GENBANK |
| Sequence 1404 | AF193795 | GENBANK |
| Sequence 1405 | AF195417 | GENBANK |
| Sequence 1406 | AF201077 | GENBANK |
| Sequence 1407 | AJ004913 | GENBANK |
| Sequence 1408 | AJ006834 | GENBANK |
| Sequence 1409 | AJ007398 | GENBANK |
| Sequence 1410 | AJ007714 | GENBANK |
| Sequence 1411 | AJ010071 | GENBANK |
| Sequence 1412 | AJ130733 | GENBANK |
| Sequence 1413 | AJ224172 | GENBANK |
| Sequence 1414 | AJ224819 | GENBANK |
| Sequence 1415 | AJ228139 | GENBANK |
| Sequence 1416 | AL035304 | GENBANK |
| Sequence 1417 | AL049233 | GENBANK |
| Sequence 1418 | AL049252 | GENBANK |
| Sequence 1419 | AL049325 | GENBANK |
| Sequence 1420 | AL049447 | GENBANK |
| Sequence 1421 | AL049969 | GENBANK |
| Sequence 1422 | AL049987 | GENBANK |
| Sequence 1423 | AL050018 | GENBANK |
| Sequence 1424 | AL050041 | GENBANK |
| Sequence 1425 | AL050089 | GENBANK |
| Sequence 1426 | AL050197 | GENBANK |
| Sequence 1427 | AL050198 | GENBANK |
| Sequence 1428 | AL050255 | GENBANK |
| Sequence 1429 | AL050265 | GENBANK |
| Sequence 1430 | AL050272 | GENBANK |
| Sequence 1431 | AL050290 | GENBANK |
| Sequence 1432 | AL050373 | GENBANK |
| Sequence 1433 | AL079298 | GENBANK |
| Sequence 1434 | AL080063 | GENBANK |
| Sequence 1435 | AL080089 | GENBANK |
| Sequence 1436 | AL080135 | GENBANK |
| Sequence 1437 | AL080144 | GENBANK |
| Sequence 1438 | AL080224 | GENBANK |
| Sequence 1439 | AL110183 | GENBANK |
| Sequence 1440 | AL117237 | GENBANK |
| Sequence 1441 | AL117412 | GENBANK |
| Sequence 1442 | AL117423 | GENBANK |
| Sequence 1443 | AL117429 | GENBANK |
| Sequence 1444 | AL117516 | GENBANK |
| Sequence 1445 | AL117543 | GENBANK |
| Sequence 1446 | AL117554 | GENBANK |
| Sequence 1447 | AL117584 | GENBANK |
| Sequence 1448 | AL117595 | GENBANK |
| Sequence 1449 | AL122079 | GENBANK |
| Sequence 1450 | D00860 | GENBANK |
| Sequence 1451 | D10040 | GENBANK |
| Sequence 1452 | D13119 | GENBANK |
| Sequence 1453 | D13286 | GENBANK |
| Sequence 1454 | D13643 | GENBANK |
| Sequence 1455 | D13645 | GENBANK |
| Sequence 1456 | D13748 | GENBANK |
| Sequence 1457 | D13757 | GENBANK |
| Sequence 1458 | D13866 | GENBANK |
| Sequence 1459 | D13900 | GENBANK |
| Sequence 1460 | D14659 | GENBANK |
| Sequence 1461 | D14697 | GENBANK |
| Sequence 1462 | D14812 | GENBANK |
| Sequence 1463 | D15057 | GENBANK |
| Sequence 1464 | D16217 | GENBANK |
| Sequence 1465 | D16480 | GENBANK |
| Sequence 1466 | D17039 | GENBANK |
| Sequence 1467 | D17652 | GENBANK |
| Sequence 1468 | D21209 | GENBANK |
| Sequence 1469 | D21262 | GENBANK |
| Sequence 1470 | D23662 | GENBANK |
| Sequence 1471 | D25542 | GENBANK |
| Sequence 1472 | D28473 | GENBANK |
| Sequence 1473 | D29954 | GENBANK |
| Sequence 1474 | D30655 | GENBANK |
| Sequence 1475 | D31767 | GENBANK |
| Sequence 1476 | D31883 | GENBANK |
| Sequence 1477 | D31884 | GENBANK |
| Sequence 1478 | D31891 | GENBANK |
| Sequence 1479 | D37991 | GENBANK |
| Sequence 1480 | D38047 | GENBANK |
| Sequence 1481 | D38293 | GENBANK |
| Sequence 1482 | D38491 | GENBANK |
| Sequence 1483 | D38521 | GENBANK |
| Sequence 1484 | D38524 | GENBANK |
| Sequence 1485 | D43948 | GENBANK |
| Sequence 1486 | D43951 | GENBANK |
| Sequence 1487 | D44466 | GENBANK |
| Sequence 1488 | D50371 | GENBANK |
| Sequence 1489 | D50372 | GENBANK |
| Sequence 1490 | D50525 | GENBANK |
| Sequence 1491 | D50645 | GENBANK |
| Sequence 1492 | D61391 | GENBANK |
| Sequence 1493 | D63878 | GENBANK |
| Sequence 1494 | D63997 | GENBANK |
| Sequence 1495 | D82345 | GENBANK |
| Sequence 1496 | D83077 | GENBANK |
| Sequence 1497 | D86326 | GENBANK |
| Sequence 1498 | D86971 | GENBANK |
| Sequence 1499 | D86974 | GENBANK |
| Sequence 1500 | D86985 | GENBANK |
| Sequence 1501 | D87078 | GENBANK |
| Sequence 1502 | D87666 | GENBANK |
| Sequence 1503 | D87667 | GENBANK |
| Sequence 1504 | D88674 | GENBANK |
| Sequence 1505 | D89053 | GENBANK |
| Sequence 1506 | D89077 | GENBANK |
| Sequence 1507 | D89667 | GENBANK |
| Sequence 1508 | E01888 | GENBANK |
| Sequence 1509 | E01956 | GENBANK |
| Sequence 1510 | E02628 | GENBANK |
| Sequence 1511 | E03569 | GENBANK |
| Sequence 1512 | E03814 | GENBANK |
| Sequence 1513 | E05957 | GENBANK |
| Sequence 1514 | E08515 | GENBANK |
| Sequence 1515 | J02959 | GENBANK |
| Sequence 1516 | J03248 | GENBANK |
| Sequence 1517 | J03799 | GENBANK |
| Sequence 1518 | J04080 | GENBANK |
| Sequence 1519 | J04973 | GENBANK |
| Sequence 1520 | J05021 | GENBANK |
| Sequence 1521 | J05192 | GENBANK |
| Sequence 1522 | K01911 | GENBANK |
| Sequence 1523 | K03002 | GENBANK |
| Sequence 1524 | L02547 | GENBANK |
| Sequence 1525 | L06070 | GENBANK |
| Sequence 1526 | L07033 | GENBANK |
| Sequence 1527 | L07261 | GENBANK |
| Sequence 1528 | L07515 | GENBANK |
| Sequence 1529 | L08441 | GENBANK |
| Sequence 1530 | L11284 | GENBANK |
| Sequence 1531 | L11667 | GENBANK |
| Sequence 1532 | L12168 | GENBANK |
| Sequence 1533 | L13806 | GENBANK |
| Sequence 1534 | L14837 | GENBANK |
| Sequence 1535 | L14848 | GENBANK |
| Sequence 1536 | L16558 | GENBANK |
| Sequence 1537 | L19184 | GENBANK |
| Sequence 1538 | L19437 | GENBANK |
| Sequence 1539 | L20688 | GENBANK |
| Sequence 1540 | L31801 | GENBANK |
| Sequence 1541 | L34840 | GENBANK |
| Sequence 1542 | L37080 | GENBANK |
| Sequence 1543 | L38486 | GENBANK |
| Sequence 1544 | L38608 | GENBANK |
| Sequence 1545 | L38951 | GENBANK |
| Sequence 1546 | L42110 | GENBANK |
| Sequence 1547 | L47647 | GENBANK |
| Sequence 1548 | L49399 | GENBANK |
| Sequence 1549 | L77701 | GENBANK |
| Sequence 1550 | M10036 | GENBANK |
| Sequence 1551 | M10119 | GENBANK |
| Sequence 1552 | M11119 | GENBANK |
| Sequence 1553 | M13231 | GENBANK |
| Sequence 1554 | M13692 | GENBANK |
| Sequence 1555 | M14200 | GENBANK |
| Sequence 1556 | M15885 | GENBANK |
| Sequence 1557 | M15887 | GENBANK |
| Sequence 1558 | M16660 | GENBANK |
| Sequence 1559 | M16804 | GENBANK |
| Sequence 1560 | M17323 | GENBANK |
| Sequence 1561 | M17885 | GENBANK |
| Sequence 1562 | M20030 | GENBANK |
| Sequence 1563 | M20372 | GENBANK |
| Sequence 1564 | M21154 | GENBANK |
| Sequence 1565 | M21895 | GENBANK |
| Sequence 1566 | M21896 | GENBANK |
| Sequence 1567 | M22382 | GENBANK |
| Sequence 1568 | M22538 | GENBANK |
| Sequence 1569 | M22590 | GENBANK |
| Sequence 1570 | M22920 | GENBANK |
| Sequence 1571 | M23613 | GENBANK |
| Sequence 1572 | M24194 | GENBANK |
| Sequence 1573 | M24902 | GENBANK |
| Sequence 1574 | M26663 | GENBANK |
| Sequence 1575 | M27335 | GENBANK |
| Sequence 1576 | M27937 | GENBANK |
| Sequence 1577 | M28016 | GENBANK |
| Sequence 1578 | M28211 | GENBANK |
| Sequence 1579 | M31627 | GENBANK |
| Sequence 1580 | M34840 | GENBANK |
| Sequence 1581 | M36072 | GENBANK |
| Sequence 1582 | M37197 | GENBANK |
| Sequence 1583 | M55421 | GENBANK |
| Sequence 1584 | M58549 | GENBANK |
| Sequence 1585 | M59849 | GENBANK |
| Sequence 1586 | M60457 | GENBANK |
| Sequence 1587 | M61916 | GENBANK |
| Sequence 1588 | M63573 | GENBANK |
| Sequence 1589 | M64347 | GENBANK |
| Sequence 1590 | M74509 | GENBANK |
| Sequence 1591 | M74777 | GENBANK |
| Sequence 1592 | M82882 | GENBANK |
| Sequence 1593 | M83822 | GENBANK |
| Sequence 1594 | M84443 | GENBANK |
| Sequence 1595 | M88163 | GENBANK |
| Sequence 1596 | M90104 | GENBANK |
| Sequence 1597 | M90309 | GENBANK |
| Sequence 1598 | M97856 | GENBANK |
| Sequence 1599 | S39329 | GENBANK |
| Sequence 1600 | S69189 | GENBANK |
| Sequence 1601 | S69272 | GENBANK |
| Sequence 1602 | S70154 | GENBANK |
| Sequence 1603 | S73591 | GENBANK |
| Sequence 1604 | S78203 | GENBANK |
| Sequence 1605 | S82081 | GENBANK |
| Sequence 1606 | S82240 | GENBANK |
| Sequence 1607 | S94541 | GENBANK |
| Sequence 1608 | U00947 | GENBANK |
| Sequence 1609 | U07919 | GENBANK |
| Sequence 1610 | U12778 | GENBANK |
| Sequence 1611 | U14971 | GENBANK |
| Sequence 1612 | U17714 | GENBANK |
| Sequence 1613 | U18914 | GENBANK |
| Sequence 1614 | U21858 | GENBANK |
| Sequence 1615 | U24169 | GENBANK |
| Sequence 1616 | U25789 | GENBANK |
| Sequence 1617 | U28249 | GENBANK |
| Sequence 1618 | U30826 | GENBANK |
| Sequence 1619 | U31905 | GENBANK |
| Sequence 1620 | U33821 | GENBANK |
| Sequence 1621 | U38654 | GENBANK |
| Sequence 1622 | U38784 | GENBANK |
| Sequence 1623 | U39360 | GENBANK |
| Sequence 1624 | U41515 | GENBANK |
| Sequence 1625 | U43188 | GENBANK |
| Sequence 1626 | U48857 | GENBANK |
| Sequence 1627 | U51004 | GENBANK |
| Sequence 1628 | U51920 | GENBANK |
| Sequence 1629 | U66469 | GENBANK |
| Sequence 1630 | U66563 | GENBANK |
| Sequence 1631 | U68140 | GENBANK |
| Sequence 1632 | U70439 | GENBANK |
| Sequence 1633 | U71363 | GENBANK |
| Sequence 1634 | U77664 | GENBANK |
| Sequence 1635 | U78516 | GENBANK |
| Sequence 1636 | U79273 | GENBANK |
| Sequence 1637 | U80034 | GENBANK |
| Sequence 1638 | U80456 | GENBANK |
| Sequence 1639 | U84720 | GENBANK |
| Sequence 1640 | U90551 | GENBANK |
| Sequence 1641 | U97280 | GENBANK |
| Sequence 1642 | X00497 | GENBANK |
| Sequence 1643 | X02544 | GENBANK |
| Sequence 1644 | X04707 | GENBANK |
| Sequence 1645 | X05332 | GENBANK |
| Sequence 1646 | X06747 | GENBANK |
| Sequence 1647 | X13238 | GENBANK |
| Sequence 1648 | X13839 | GENBANK |
| Sequence 1649 | X15183 | GENBANK |
| Sequence 1650 | X15187 | GENBANK |
| Sequence 1651 | X15722 | GENBANK |
| Sequence 1652 | X15729 | GENBANK |
| Sequence 1653 | X17620 | GENBANK |
| Sequence 1654 | X52943 | GENBANK |
| Sequence 1655 | X53280 | GENBANK |
| Sequence 1656 | X53605 | GENBANK |
| Sequence 1657 | X56998 | GENBANK |
| Sequence 1658 | X59405 | GENBANK |
| Sequence 1659 | X60656 | GENBANK |
| Sequence 1660 | X60708 | GENBANK |
| Sequence 1661 | X61100 | GENBANK |
| Sequence 1662 | X66276 | GENBANK |
| Sequence 1663 | X69086 | GENBANK |
| Sequence 1664 | X73114 | GENBANK |
| Sequence 1665 | X74070 | GENBANK |
| Sequence 1666 | X74331 | GENBANK |
| Sequence 1667 | X75861 | GENBANK |
| Sequence 1668 | X76013 | GENBANK |
| Sequence 1669 | X76732 | GENBANK |
| Sequence 1670 | X81900 | GENBANK |
| Sequence 1671 | X82125 | GENBANK |
| Sequence 1672 | X82157 | GENBANK |
| Sequence 1673 | X90583 | GENBANK |
| Sequence 1674 | X94323 | GENBANK |
| Sequence 1675 | X95240 | GENBANK |
| Sequence 1676 | X95325 | GENBANK |
| Sequence 1677 | X97335 | GENBANK |
| Sequence 1678 | X98743 | GENBANK |
| Sequence 1679 | Y00052 | GENBANK |
| Sequence 1680 | Y00282 | GENBANK |
| Sequence 1681 | Y08915 | GENBANK |
| Sequence 1682 | Y08991 | GENBANK |
| Sequence 1683 | Y12860 | GENBANK |
| Sequence 1684 | Y13286 | GENBANK |
| Sequence 1685 | Z11227 | GENBANK |
| Sequence 1686 | Z26248 | GENBANK |
| Sequence 1687 | Z29067 | GENBANK |
| Sequence 1688 | Z31696 | GENBANK |
| Sequence 1689 | Z36819 | GENBANK |
| Sequence 1690 | Z48633 | GENBANK |
| Sequence 1691 | Z68747 | GENBANK |
| Sequence 1692 | Z69043 | GENBANK |
| Sequence 1693 | AA001319 | dbEST |
| Sequence 1694 | AA001792 | dbEST |
| Sequence 1695 | AA002181 | dbEST |
| Sequence 1696 | AA004609 | dbEST |
| Sequence 1697 | AA010480 | dbEST |
| Sequence 1698 | AA010828 | dbEST |
| Sequence 1699 | AA013090 | dbEST |
| Sequence 1700 | AA017469 | dbEST |
| Sequence 1701 | AA017500 | dbEST |
| Sequence 1702 | AA018347 | dbEST |
| Sequence 1703 | AA025373 | dbEST |
| Sequence 1704 | AA026983 | dbEST |
| Sequence 1705 | AA027862 | dbEST |
| Sequence 1706 | AA029889 | dbEST |
| Sequence 1707 | AA029947 | dbEST |
| Sequence 1708 | AA031293 | dbEST |
| Sequence 1709 | AA031762 | dbEST |
| Sequence 1710 | AA032221 | dbEST |
| Sequence 1711 | AA033869 | dbEST |
| Sequence 1712 | AA034186 | dbEST |
| Sequence 1713 | AA034516 | dbEST |
| Sequence 1714 | AA035001 | dbEST |
| Sequence 1715 | AA035090 | dbEST |
| Sequence 1716 | AA037138 | dbEST |
| Sequence 1717 | AA037216 | dbEST |
| Sequence 1718 | AA037828 | dbEST |
| Sequence 1719 | AA037877 | dbEST |
| Sequence 1720 | AA040037 | dbEST |
| Sequence 1721 | AA040820 | dbEST |
| Sequence 1722 | AA044591 | dbEST |
| Sequence 1723 | AA046808 | dbEST |
| Sequence 1724 | AA046973 | dbEST |
| Sequence 1725 | AA047729 | dbEST |
| Sequence 1726 | AA053522 | dbEST |
| Sequence 1727 | AA054120 | dbEST |
| Sequence 1728 | AA056034 | dbEST |
| Sequence 1729 | AA056482 | dbEST |
| Sequence 1730 | AA063501 | dbEST |
| Sequence 1731 | AA069079 | dbEST |
| Sequence 1732 | AA069693 | dbEST |
| Sequence 1733 | AA069784 | dbEST |
| Sequence 1734 | AA071084 | dbEST |
| Sequence 1735 | AA074818 | dbEST |
| Sequence 1736 | AA075165 | dbEST |
| Sequence 1737 | AA075257 | dbEST |
| Sequence 1738 | AA075530 | dbEST |
| Sequence 1739 | AA077906 | dbEST |
| Sequence 1740 | AA078904 | dbEST |
| Sequence 1741 | AA080889 | dbEST |
| Sequence 1742 | AA081751 | dbEST |
| Sequence 1743 | AA082087 | dbEST |
| Sequence 1744 | AA082754 | dbEST |
| Sequence 1745 | AA083096 | dbEST |
| Sequence 1746 | AA083410 | dbEST |
| Sequence 1747 | AA085459 | dbEST |
| Sequence 1748 | AA085632 | dbEST |
| Sequence 1749 | AA088187 | dbEST |
| Sequence 1750 | AA088372 | dbEST |
| Sequence 1751 | AA088755 | dbEST |
| Sequence 1752 | AA093974 | dbEST |
| Sequence 1753 | AA094656 | dbEST |
| Sequence 1754 | AA095478 | dbEST |
| Sequence 1755 | AA098876 | dbEST |
| Sequence 1756 | AA099080 | dbEST |
| Sequence 1757 | AA099602 | dbEST |
| Sequence 1758 | AA099976 | dbEST |
| Sequence 1759 | AA100707 | dbEST |
| Sequence 1760 | AA100885 | dbEST |
| Sequence 1761 | AA101237 | dbEST |
| Sequence 1762 | AA102605 | dbEST |
| Sequence 1763 | AA114945 | dbEST |
| Sequence 1764 | AA114952 | dbEST |
| Sequence 1765 | AA115021 | dbEST |
| Sequence 1766 | AA115492 | dbEST |
| Sequence 1767 | AA115604 | dbEST |
| Sequence 1768 | AA120852 | dbEST |
| Sequence 1769 | AA122129 | dbEST |
| Sequence 1770 | AA126025 | dbEST |
| Sequence 1771 | AA126373 | dbEST |
| Sequence 1772 | AA127336 | dbEST |
| Sequence 1773 | AA127369 | dbEST |
| Sequence 1774 | AA128114 | dbEST |
| Sequence 1775 | AA130162 | dbEST |
| Sequence 1776 | AA130229 | dbEST |
| Sequence 1777 | AA130262 | dbEST |
| Sequence 1778 | AA130784 | dbEST |
| Sequence 1779 | AA131198 | dbEST |
| Sequence 1780 | AA131749 | dbEST |
| Sequence 1781 | AA131961 | dbEST |
| Sequence 1782 | AA132508 | dbEST |
| Sequence 1783 | AA132633 | dbEST |
| Sequence 1784 | AA132992 | dbEST |
| Sequence 1785 | AA133692 | dbEST |
| Sequence 1786 | AA134266 | dbEST |
| Sequence 1787 | AA134267 | dbEST |
| Sequence 1788 | AA134527 | dbEST |
| Sequence 1789 | AA135875 | dbEST |
| Sequence 1790 | AA136351 | dbEST |
| Sequence 1791 | AA136874 | dbEST |
| Sequence 1792 | AA143438 | dbEST |
| Sequence 1793 | AA143634 | dbEST |
| Sequence 1794 | AA146589 | dbEST |
| Sequence 1795 | AA147507 | dbEST |
| Sequence 1796 | AA148019 | dbEST |
| Sequence 1797 | AA148087 | dbEST |
| Sequence 1798 | AA156782 | dbEST |
| Sequence 1799 | AA156862 | dbEST |
| Sequence 1800 | AA157741 | dbEST |
| Sequence 1801 | AA159774 | dbEST |
| Sequence 1802 | AA159849 | dbEST |
| Sequence 1803 | AA159910 | dbEST |
| Sequence 1804 | AA165027 | dbEST |
| Sequence 1805 | AA165290 | dbEST |
| Sequence 1806 | AA165593 | dbEST |
| Sequence 1807 | AA166952 | dbEST |
| Sequence 1808 | AA167084 | dbEST |
| Sequence 1809 | AA167766 | dbEST |
| Sequence 1810 | AA167826 | dbEST |
| Sequence 1811 | AA169520 | dbEST |
| Sequence 1812 | AA169736 | dbEST |
| Sequence 1813 | AA173331 | dbEST |
| Sequence 1814 | AA173998 | dbEST |
| Sequence 1815 | AA180137 | dbEST |
| Sequence 1816 | AA182948 | dbEST |
| Sequence 1817 | AA187288 | dbEST |
| Sequence 1818 | AA187809 | dbEST |
| Sequence 1819 | AA188051 | dbEST |
| Sequence 1820 | AA188140 | dbEST |
| Sequence 1821 | AA190852 | dbEST |
| Sequence 1822 | AA190914 | dbEST |
| Sequence 1823 | AA191661 | dbEST |
| Sequence 1824 | AA193247 | dbEST |
| Sequence 1825 | AA195617 | dbEST |
| Sequence 1826 | AA195831 | dbEST |
| Sequence 1827 | AA196978 | dbEST |
| Sequence 1828 | AA203492 | dbEST |
| Sequence 1829 | AA204854 | dbEST |
| Sequence 1830 | AA205647 | dbEST |
| Sequence 1831 | AA205870 | dbEST |
| Sequence 1832 | AA206137 | dbEST |
| Sequence 1833 | AA206214 | dbEST |
| Sequence 1834 | AA207266 | dbEST |
| Sequence 1835 | AA210741 | dbEST |
| Sequence 1836 | AA210833 | dbEST |
| Sequence 1837 | AA210840 | dbEST |
| Sequence 1838 | AA218536 | dbEST |
| Sequence 1839 | AA223774 | dbEST |
| Sequence 1840 | AA224132 | dbEST |
| Sequence 1841 | AA224751 | dbEST |
| Sequence 1842 | AA224876 | dbEST |
| Sequence 1843 | AA225002 | dbEST |
| Sequence 1844 | AA225458 | dbEST |
| Sequence 1845 | AA225857 | dbEST |
| Sequence 1846 | AA226171 | dbEST |
| Sequence 1847 | AA226275 | dbEST |
| Sequence 1848 | AA226321 | dbEST |
| Sequence 1849 | AA226611 | dbEST |
| Sequence 1850 | AA226709 | dbEST |
| Sequence 1851 | AA227873 | dbEST |
| Sequence 1852 | AA228440 | dbEST |
| Sequence 1853 | AA229199 | dbEST |
| Sequence 1854 | AA229424 | dbEST |
| Sequence 1855 | AA229611 | dbEST |
| Sequence 1856 | AA230169 | dbEST |
| Sequence 1857 | AA232734 | dbEST |
| Sequence 1858 | AA232843 | dbEST |
| Sequence 1859 | AA233428 | dbEST |
| Sequence 1860 | AA234464 | dbEST |
| Sequence 1861 | AA234818 | dbEST |
| Sequence 1862 | AA235370 | dbEST |
| Sequence 1863 | AA235802 | dbEST |
| Sequence 1864 | AA236667 | dbEST |
| Sequence 1865 | AA236904 | dbEST |
| Sequence 1866 | AA242864 | dbEST |
| Sequence 1867 | AA243230 | dbEST |
| Sequence 1868 | AA243980 | dbEST |
| Sequence 1869 | AA244052 | dbEST |
| Sequence 1870 | AA249154 | dbEST |
| Sequence 1871 | AA251321 | dbEST |
| Sequence 1872 | AA253384 | dbEST |
| Sequence 1873 | AA256591 | dbEST |
| Sequence 1874 | AA258357 | dbEST |
| Sequence 1875 | AA259062 | dbEST |
| Sequence 1876 | AA261873 | dbEST |
| Sequence 1877 | AA262345 | dbEST |
| Sequence 1878 | AA278956 | dbEST |
| Sequence 1879 | AA280848 | dbEST |
| Sequence 1880 | AA283904 | dbEST |
| Sequence 1881 | AA284835 | dbEST |
| Sequence 1882 | AA284971 | dbEST |
| Sequence 1883 | AA285040 | dbEST |
| Sequence 1884 | AA285290 | dbEST |
| Sequence 1885 | AA290591 | dbEST |
| Sequence 1886 | AA291680 | dbEST |
| Sequence 1887 | AA292131 | dbEST |
| Sequence 1888 | AA292866 | dbEST |
| Sequence 1889 | AA293802 | dbEST |
| Sequence 1890 | AA296465 | dbEST |
| Sequence 1891 | AA297215 | dbEST |
| Sequence 1892 | AA297512 | dbEST |
| Sequence 1893 | AA298596 | dbEST |
| Sequence 1894 | AA298786 | dbEST |
| Sequence 1895 | AA298794 | dbEST |
| Sequence 1896 | AA299594 | dbEST |
| Sequence 1897 | AA300728 | dbEST |
| Sequence 1898 | AA301391 | dbEST |
| Sequence 1899 | AA301907 | dbEST |
| Sequence 1900 | AA303172 | dbEST |
| Sequence 1901 | AA303682 | dbEST |
| Sequence 1902 | AA303796 | dbEST |
| Sequence 1903 | AA303880 | dbEST |
| Sequence 1904 | AA304947 | dbEST |
| Sequence 1905 | AA305193 | dbEST |
| Sequence 1906 | AA305315 | dbEST |
| Sequence 1907 | AA305331 | dbEST |
| Sequence 1908 | AA305409 | dbEST |
| Sequence 1909 | AA305526 | dbEST |
| Sequence 1910 | AA305566 | dbEST |
| Sequence 1911 | AA306540 | dbEST |
| Sequence 1912 | AA306812 | dbEST |
| Sequence 1913 | AA307697 | dbEST |
| Sequence 1914 | AA307720 | dbEST |
| Sequence 1915 | AA307849 | dbEST |
| Sequence 1916 | AA308051 | dbEST |
| Sequence 1917 | AA308273 | dbEST |
| Sequence 1918 | AA308332 | dbEST |
| Sequence 1919 | AA310057 | dbEST |
| Sequence 1920 | AA311505 | dbEST |
| Sequence 1921 | AA312652 | dbEST |
| Sequence 1922 | AA313170 | dbEST |
| Sequence 1923 | AA313534 | dbEST |
| Sequence 1924 | AA313779 | dbEST |
| Sequence 1925 | AA314350 | dbEST |
| Sequence 1926 | AA314599 | dbEST |
| Sequence 1927 | AA314673 | dbEST |
| Sequence 1928 | AA315049 | dbEST |
| Sequence 1929 | AA315189 | dbEST |
| Sequence 1930 | AA315311 | dbEST |
| Sequence 1931 | AA316181 | dbEST |
| Sequence 1932 | AA316632 | dbEST |
| Sequence 1933 | AA317952 | dbEST |
| Sequence 1934 | AA318100 | dbEST |
| Sequence 1935 | AA320611 | dbEST |
| Sequence 1936 | AA322728 | dbEST |
| Sequence 1937 | AA322922 | dbEST |
| Sequence 1938 | AA324683 | dbEST |
| Sequence 1939 | AA325108 | dbEST |
| Sequence 1940 | AA325220 | dbEST |
| Sequence 1941 | AA329110 | dbEST |
| Sequence 1942 | AA331359 | dbEST |
| Sequence 1943 | AA332139 | dbEST |
| Sequence 1944 | AA332551 | dbEST |
| Sequence 1945 | AA336798 | dbEST |
| Sequence 1946 | AA340632 | dbEST |
| Sequence 1947 | AA342231 | dbEST |
| Sequence 1948 | AA344376 | dbEST |
| Sequence 1949 | AA345904 | dbEST |
| Sequence 1950 | AA347340 | dbEST |
| Sequence 1951 | AA351799 | dbEST |
| Sequence 1952 | AA354915 | dbEST |
| Sequence 1953 | AA355119 | dbEST |
| Sequence 1954 | AA357593 | dbEST |
| Sequence 1955 | AA358169 | dbEST |
| Sequence 1956 | AA360190 | dbEST |
| Sequence 1957 | AA361997 | dbEST |
| Sequence 1958 | AA362731 | dbEST |
| Sequence 1959 | AA365229 | dbEST |
| Sequence 1960 | AA366269 | dbEST |
| Sequence 1961 | AA369521 | dbEST |
| Sequence 1962 | AA370110 | dbEST |
| Sequence 1963 | AA370122 | dbEST |
| Sequence 1964 | AA370337 | dbEST |
| Sequence 1965 | AA373079 | dbEST |
| Sequence 1966 | AA374718 | dbEST |
| Sequence 1967 | AA375298 | dbEST |
| Sequence 1968 | AA375312 | dbEST |
| Sequence 1969 | AA376292 | dbEST |
| Sequence 1970 | AA382481 | dbEST |
| Sequence 1971 | AA384322 | dbEST |
| Sequence 1972 | AA384529 | dbEST |
| Sequence 1973 | AA384756 | dbEST |
| Sequence 1974 | AA385573 | dbEST |
| Sequence 1975 | AA386056 | dbEST |
| Sequence 1976 | AA386087 | dbEST |
| Sequence 1977 | AA393080 | dbEST |
| Sequence 1978 | AA393699 | dbEST |
| Sequence 1979 | AA397460 | dbEST |
| Sequence 1980 | AA398619 | dbEST |
| Sequence 1981 | AA400692 | dbEST |
| Sequence 1982 | AA402000 | dbEST |
| Sequence 1983 | AA402238 | dbEST |
| Sequence 1984 | AA404669 | dbEST |
| Sequence 1985 | AA406168 | dbEST |
| Sequence 1986 | AA406191 | dbEST |
| Sequence 1987 | AA418628 | dbEST |
| Sequence 1988 | AA418685 | dbEST |
| Sequence 1989 | AA418824 | dbEST |
| Sequence 1990 | AA419011 | dbEST |
| Sequence 1991 | AA420625 | dbEST |
| Sequence 1992 | AA420645 | dbEST |
| Sequence 1993 | AA420721 | dbEST |
| Sequence 1994 | AA420826 | dbEST |
| Sequence 1995 | AA423957 | dbEST |
| Sequence 1996 | AA424109 | dbEST |
| Sequence 1997 | AA424202 | dbEST |
| Sequence 1998 | AA424466 | dbEST |
| Sequence 1999 | AA424543 | dbEST |
| Sequence 2000 | AA424648 | dbEST |
| Sequence 2001 | AA426209 | dbEST |
| Sequence 2002 | AA427895 | dbEST |
| Sequence 2003 | AA428143 | dbEST |
| Sequence 2004 | AA429173 | dbEST |
| Sequence 2005 | AA429190 | dbEST |
| Sequence 2006 | AA429336 | dbEST |
| Sequence 2007 | AA429381 | dbEST |
| Sequence 2008 | AA430304 | dbEST |
| Sequence 2009 | AA432203 | dbEST |
| Sequence 2010 | AA434502 | dbEST |
| Sequence 2011 | AA436395 | dbEST |
| Sequence 2012 | AA436730 | dbEST |
| Sequence 2013 | AA437224 | dbEST |
| Sequence 2014 | AA442145 | dbEST |
| Sequence 2015 | AA442287 | dbEST |
| Sequence 2016 | AA442415 | dbEST |
| Sequence 2017 | AA447069 | dbEST |
| Sequence 2018 | AA447254 | dbEST |
| Sequence 2019 | AA447322 | dbEST |
| Sequence 2020 | AA447343 | dbEST |
| Sequence 2021 | AA448036 | dbEST |
| Sequence 2022 | AA448195 | dbEST |
| Sequence 2023 | AA448614 | dbEST |
| Sequence 2024 | AA449345 | dbEST |
| Sequence 2025 | AA449889 | dbEST |
| Sequence 2026 | AA451633 | dbEST |
| Sequence 2027 | AA453310 | dbEST |
| Sequence 2028 | AA453562 | dbEST |
| Sequence 2029 | AA453924 | dbEST |
| Sequence 2030 | AA455235 | dbEST |
| Sequence 2031 | AA456344 | dbEST |
| Sequence 2032 | AA458516 | dbEST |
| Sequence 2033 | AA459321 | dbEST |
| Sequence 2034 | AA459517 | dbEST |
| Sequence 2035 | AA461128 | dbEST |
| Sequence 2036 | AA464133 | dbEST |
| Sequence 2037 | AA464338 | dbEST |
| Sequence 2038 | AA464568 | dbEST |
| Sequence 2039 | AA468575 | dbEST |
| Sequence 2040 | AA468839 | dbEST |
| Sequence 2041 | AA469199 | dbEST |
| Sequence 2042 | AA469406 | dbEST |
| Sequence 2043 | AA476263 | dbEST |
| Sequence 2044 | AA477173 | dbEST |
| Sequence 2045 | AA477698 | dbEST |
| Sequence 2046 | AA479614 | dbEST |
| Sequence 2047 | AA480420 | dbEST |
| Sequence 2048 | AA480505 | dbEST |
| Sequence 2049 | AA481355 | dbEST |
| Sequence 2050 | AA482853 | dbEST |
| Sequence 2051 | AA483565 | dbEST |
| Sequence 2052 | AA485467 | dbEST |
| Sequence 2053 | AA485767 | dbEST |
| Sequence 2054 | AA487503 | dbEST |
| Sequence 2055 | AA488280 | dbEST |
| Sequence 2056 | AA489032 | dbEST |
| Sequence 2057 | AA489410 | dbEST |
| Sequence 2058 | AA489921 | dbEST |
| Sequence 2059 | AA491081 | dbEST |
| Sequence 2060 | AA491866 | dbEST |
| Sequence 2061 | AA492026 | dbEST |
| Sequence 2062 | AA492143 | dbEST |
| Sequence 2063 | AA492280 | dbEST |
| Sequence 2064 | AA493512 | dbEST |
| Sequence 2065 | AA493742 | dbEST |
| Sequence 2066 | AA493962 | dbEST |
| Sequence 2067 | AA494344 | dbEST |
| Sequence 2068 | AA501752 | dbEST |
| Sequence 2069 | AA501945 | dbEST |
| Sequence 2070 | AA502320 | dbEST |
| Sequence 2071 | AA502979 | dbEST |
| Sequence 2072 | AA503115 | dbEST |
| Sequence 2073 | AA503224 | dbEST |
| Sequence 2074 | AA503330 | dbEST |
| Sequence 2075 | AA503611 | dbEST |
| Sequence 2076 | AA504134 | dbEST |
| Sequence 2077 | AA504265 | dbEST |
| Sequence 2078 | AA504343 | dbEST |
| Sequence 2079 | AA505124 | dbEST |
| Sequence 2080 | AA505625 | dbEST |
| Sequence 2081 | AA506099 | dbEST |
| Sequence 2082 | AA507505 | dbEST |
| Sequence 2083 | AA508804 | dbEST |
| Sequence 2084 | AA513597 | dbEST |
| Sequence 2085 | AA513940 | dbEST |
| Sequence 2086 | AA514490 | dbEST |
| Sequence 2087 | AA514775 | dbEST |
| Sequence 2088 | AA514991 | dbEST |
| Sequence 2089 | AA516531 | dbEST |
| Sequence 2090 | AA521142 | dbEST |
| Sequence 2091 | AA522849 | dbEST |
| Sequence 2092 | AA522850 | dbEST |
| Sequence 2093 | AA523252 | dbEST |
| Sequence 2094 | AA523498 | dbEST |
| Sequence 2095 | AA523751 | dbEST |
| Sequence 2096 | AA524064 | dbEST |
| Sequence 2097 | AA524258 | dbEST |
| Sequence 2098 | AA524285 | dbEST |
| Sequence 2099 | AA525269 | dbEST |
| Sequence 2100 | AA526403 | dbEST |
| Sequence 2101 | AA526430 | dbEST |
| Sequence 2102 | AA526834 | dbEST |
| Sequence 2103 | AA526869 | dbEST |
| Sequence 2104 | AA527342 | dbEST |
| Sequence 2105 | AA527730 | dbEST |
| Sequence 2106 | AA527805 | dbEST |
| Sequence 2107 | AA531068 | dbEST |
| Sequence 2108 | AA531487 | dbEST |
| Sequence 2109 | AA531563 | dbEST |
| Sequence 2110 | AA532398 | dbEST |
| Sequence 2111 | AA532934 | dbEST |
| Sequence 2112 | AA533575 | dbEST |
| Sequence 2113 | AA533963 | dbEST |
| Sequence 2114 | AA534171 | dbEST |
| Sequence 2115 | AA534173 | dbEST |
| Sequence 2116 | AA534298 | dbEST |
| Sequence 2117 | AA534395 | dbEST |
| Sequence 2118 | AA536129 | dbEST |
| Sequence 2119 | AA541515 | dbEST |
| Sequence 2120 | AA541677 | dbEST |
| Sequence 2121 | AA553726 | dbEST |
| Sequence 2122 | AA553826 | dbEST |
| Sequence 2123 | AA554326 | dbEST |
| Sequence 2124 | AA557174 | dbEST |
| Sequence 2125 | AA557683 | dbEST |
| Sequence 2126 | AA558280 | dbEST |
| Sequence 2127 | AA558547 | dbEST |
| Sequence 2128 | AA558755 | dbEST |
| Sequence 2129 | AA559055 | dbEST |
| Sequence 2130 | AA564119 | dbEST |
| Sequence 2131 | AA565420 | dbEST |
| Sequence 2132 | AA568138 | dbEST |
| Sequence 2133 | AA568990 | dbEST |
| Sequence 2134 | AA569765 | dbEST |
| Sequence 2135 | AA570519 | dbEST |
| Sequence 2136 | AA570665 | dbEST |
| Sequence 2137 | AA573975 | dbEST |
| Sequence 2138 | AA574223 | dbEST |
| Sequence 2139 | AA574397 | dbEST |
| Sequence 2140 | AA575933 | dbEST |
| Sequence 2141 | AA575935 | dbEST |
| Sequence 2142 | AA577800 | dbEST |
| Sequence 2143 | AA578577 | dbEST |
| Sequence 2144 | AA578773 | dbEST |
| Sequence 2145 | AA578904 | dbEST |
| Sequence 2146 | AA579617 | dbEST |
| Sequence 2147 | AA580279 | dbEST |
| Sequence 2148 | AA580399 | dbEST |
| Sequence 2149 | AA583449 | dbEST |
| Sequence 2150 | AA585110 | dbEST |
| Sequence 2151 | AA587468 | dbEST |
| Sequence 2152 | AA588848 | dbEST |
| Sequence 2153 | AA592908 | dbEST |
| Sequence 2154 | AA593385 | dbEST |
| Sequence 2155 | AA599669 | dbEST |
| Sequence 2156 | AA602961 | dbEST |
| Sequence 2157 | AA604619 | dbEST |
| Sequence 2158 | AA609394 | dbEST |
| Sequence 2159 | AA609641 | dbEST |
| Sequence 2160 | AA610581 | dbEST |
| Sequence 2161 | AA612573 | dbEST |
| Sequence 2162 | AA612864 | dbEST |
| Sequence 2163 | AA613891 | dbEST |
| Sequence 2164 | AA614350 | dbEST |
| Sequence 2165 | AA620396 | dbEST |
| Sequence 2166 | AA620750 | dbEST |
| Sequence 2167 | AA622765 | dbEST |
| Sequence 2168 | AA627855 | dbEST |
| Sequence 2169 | AA630303 | dbEST |
| Sequence 2170 | AA631303 | dbEST |
| Sequence 2171 | AA631419 | dbEST |
| Sequence 2172 | AA631460 | dbEST |
| Sequence 2173 | AA631868 | dbEST |
| Sequence 2174 | AA634772 | dbEST |
| Sequence 2175 | AA635024 | dbEST |
| Sequence 2176 | AA639902 | dbEST |
| Sequence 2177 | AA640038 | dbEST |
| Sequence 2178 | AA640474 | dbEST |
| Sequence 2179 | AA641195 | dbEST |
| Sequence 2180 | AA644237 | dbEST |
| Sequence 2181 | AA648318 | dbEST |
| Sequence 2182 | AA648757 | dbEST |
| Sequence 2183 | AA649012 | dbEST |
| Sequence 2184 | AA650114 | dbEST |
| Sequence 2185 | AA652167 | dbEST |
| Sequence 2186 | AA652478 | dbEST |
| Sequence 2187 | AA653601 | dbEST |
| Sequence 2188 | AA654875 | dbEST |
| Sequence 2189 | AA657411 | dbEST |
| Sequence 2190 | AA657488 | dbEST |
| Sequence 2191 | AA659388 | dbEST |
| Sequence 2192 | AA659719 | dbEST |
| Sequence 2193 | AA664480 | dbEST |
| Sequence 2194 | AA665209 | dbEST |
| Sequence 2195 | AA677531 | dbEST |
| Sequence 2196 | AA679791 | dbEST |
| Sequence 2197 | AA679935 | dbEST |
| Sequence 2198 | AA682244 | dbEST |
| Sequence 2199 | AA682252 | dbEST |
| Sequence 2200 | AA682283 | dbEST |
| Sequence 2201 | AA687216 | dbEST |
| Sequence 2202 | AA687336 | dbEST |
| Sequence 2203 | AA700232 | dbEST |
| Sequence 2204 | AA700876 | dbEST |
| Sequence 2205 | AA702888 | dbEST |
| Sequence 2206 | AA703396 | dbEST |
| Sequence 2207 | AA705433 | dbEST |
| Sequence 2208 | AA706764 | dbEST |
| Sequence 2209 | AA707607 | dbEST |
| Sequence 2210 | AA720578 | dbEST |
| Sequence 2211 | AA721375 | dbEST |
| Sequence 2212 | AA723525 | dbEST |
| Sequence 2213 | AA725400 | dbEST |
| Sequence 2214 | AA730534 | dbEST |
| Sequence 2215 | AA730780 | dbEST |
| Sequence 2216 | AA731556 | dbEST |
| Sequence 2217 | AA731700 | dbEST |
| Sequence 2218 | AA741297 | dbEST |
| Sequence 2219 | AA743975 | dbEST |
| Sequence 2220 | AA745694 | dbEST |
| Sequence 2221 | AA749045 | dbEST |
| Sequence 2222 | AA758348 | dbEST |
| Sequence 2223 | AA760738 | dbEST |
| Sequence 2224 | AA760894 | dbEST |
| Sequence 2225 | AA767154 | dbEST |
| Sequence 2226 | AA767457 | dbEST |
| Sequence 2227 | AA767764 | dbEST |
| Sequence 2228 | AA768348 | dbEST |
| Sequence 2229 | AA769434 | dbEST |
| Sequence 2230 | AA769696 | dbEST |
| Sequence 2231 | AA769722 | dbEST |
| Sequence 2232 | AA774869 | dbEST |
| Sequence 2233 | AA775918 | dbEST |
| Sequence 2234 | AA776740 | dbEST |
| Sequence 2235 | AA779728 | dbEST |
| Sequence 2236 | AA788779 | dbEST |
| Sequence 2237 | AA788875 | dbEST |
| Sequence 2238 | AA804544 | dbEST |
| Sequence 2239 | AA805228 | dbEST |
| Sequence 2240 | AA808492 | dbEST |
| Sequence 2241 | AA815305 | dbEST |
| Sequence 2242 | AA824401 | dbEST |
| Sequence 2243 | AA825768 | dbEST |
| Sequence 2244 | AA825940 | dbEST |
| Sequence 2245 | AA827875 | dbEST |
| Sequence 2246 | AA827878 | dbEST |
| Sequence 2247 | AA829236 | dbEST |
| Sequence 2248 | AA829547 | dbEST |
| Sequence 2249 | AA834540 | dbEST |
| Sequence 2250 | AA838003 | dbEST |
| Sequence 2251 | AA838410 | dbEST |
| Sequence 2252 | AA838681 | dbEST |
| Sequence 2253 | AA843326 | dbEST |
| Sequence 2254 | AA843592 | dbEST |
| Sequence 2255 | AA843706 | dbEST |
| Sequence 2256 | AA844070 | dbEST |
| Sequence 2257 | AA847656 | dbEST |
| Sequence 2258 | AA848129 | dbEST |
| Sequence 2259 | AA853183 | dbEST |
| Sequence 2260 | AA856719 | dbEST |
| Sequence 2261 | AA857476 | dbEST |
| Sequence 2262 | AA857936 | dbEST |
| Sequence 2263 | AA872606 | dbEST |
| Sequence 2264 | AA873182 | dbEST |
| Sequence 2265 | AA876668 | dbEST |
| Sequence 2266 | AA876897 | dbEST |
| Sequence 2267 | AA877789 | dbEST |
| Sequence 2268 | AA878212 | dbEST |
| Sequence 2269 | AA883580 | dbEST |
| Sequence 2270 | AA883901 | dbEST |
| Sequence 2271 | AA884098 | dbEST |
| Sequence 2272 | AA886818 | dbEST |
| Sequence 2273 | AA888589 | dbEST |
| Sequence 2274 | AA888665 | dbEST |
| Sequence 2275 | AA902388 | dbEST |
| Sequence 2276 | AA907575 | dbEST |
| Sequence 2277 | AA912796 | dbEST |
| Sequence 2278 | AA916635 | dbEST |
| Sequence 2279 | AA916753 | dbEST |
| Sequence 2280 | AA918023 | dbEST |
| Sequence 2281 | AA922237 | dbEST |
| Sequence 2282 | AA923278 | dbEST |
| Sequence 2283 | AA926992 | dbEST |
| Sequence 2284 | AA932252 | dbEST |
| Sequence 2285 | AA934368 | dbEST |
| Sequence 2286 | AA934755 | dbEST |
| Sequence 2287 | AA935526 | dbEST |
| Sequence 2288 | AA935811 | dbEST |
| Sequence 2289 | AA939199 | dbEST |
| Sequence 2290 | AA939266 | dbEST |
| Sequence 2291 | AA948320 | dbEST |
| Sequence 2292 | AA954674 | dbEST |
| Sequence 2293 | AA962300 | dbEST |
| Sequence 2294 | AA968621 | dbEST |
| Sequence 2295 | AA968756 | dbEST |
| Sequence 2296 | AA969459 | dbEST |
| Sequence 2297 | AA969512 | dbEST |
| Sequence 2298 | AA973560 | dbEST |
| Sequence 2299 | AA977197 | dbEST |
| Sequence 2300 | AA977226 | dbEST |
| Sequence 2301 | AA978214 | dbEST |
| Sequence 2302 | AA983560 | dbEST |
| Sequence 2303 | AA987290 | dbEST |
| Sequence 2304 | AA991398 | dbEST |
| Sequence 2305 | AA995066 | dbEST |
| Sequence 2306 | AA995747 | dbEST |
| Sequence 2307 | AA996010 | dbEST |
| Sequence 2308 | AF034176 | dbEST |
| Sequence 2309 | AF063521 | dbEST |
| Sequence 2310 | AI015790 | dbEST |
| Sequence 2311 | AI018625 | dbEST |
| Sequence 2312 | AI022687 | dbEST |
| Sequence 2313 | AI024549 | dbEST |
| Sequence 2314 | AI025419 | dbEST |
| Sequence 2315 | AI026767 | dbEST |
| Sequence 2316 | AI027536 | dbEST |
| Sequence 2317 | AI032392 | dbEST |
| Sequence 2318 | AI032838 | dbEST |
| Sequence 2319 | AI038692 | dbEST |
| Sequence 2320 | AI040598 | dbEST |
| Sequence 2321 | AI050871 | dbEST |
| Sequence 2322 | AI051210 | dbEST |
| Sequence 2323 | AI052333 | dbEST |
| Sequence 2324 | AI055939 | dbEST |
| Sequence 2325 | AI061649 | dbEST |
| Sequence 2326 | AI064691 | dbEST |
| Sequence 2327 | AI064844 | dbEST |
| Sequence 2328 | AI065092 | dbEST |
| Sequence 2329 | AI073465 | dbEST |
| Sequence 2330 | AI075324 | dbEST |
| Sequence 2331 | AI079558 | dbEST |
| Sequence 2332 | AI080267 | dbEST |
| Sequence 2333 | AI080480 | dbEST |
| Sequence 2334 | AI082168 | dbEST |
| Sequence 2335 | AI083814 | dbEST |
| Sequence 2336 | AI084604 | dbEST |
| Sequence 2337 | AI085165 | dbEST |
| Sequence 2338 | AI087145 | dbEST |
| Sequence 2339 | AI087888 | dbEST |
| Sequence 2340 | AI088778 | dbEST |
| Sequence 2341 | AI090809 | dbEST |
| Sequence 2342 | AI092479 | dbEST |
| Sequence 2343 | AI094741 | dbEST |
| Sequence 2344 | AI097410 | dbEST |
| Sequence 2345 | AI114602 | dbEST |
| Sequence 2346 | AI114651 | dbEST |
| Sequence 2347 | AI126233 | dbEST |
| Sequence 2348 | AI126867 | dbEST |
| Sequence 2349 | AI128828 | dbEST |
| Sequence 2350 | AI130796 | dbEST |
| Sequence 2351 | AI133466 | dbEST |
| Sequence 2352 | AI133690 | dbEST |
| Sequence 2353 | AI138633 | dbEST |
| Sequence 2354 | AI138640 | dbEST |
| Sequence 2355 | AI140096 | dbEST |
| Sequence 2356 | AI141770 | dbEST |
| Sequence 2357 | AI142083 | dbEST |
| Sequence 2358 | AI143775 | dbEST |
| Sequence 2359 | AI149592 | dbEST |
| Sequence 2360 | AI151140 | dbEST |
| Sequence 2361 | AI161068 | dbEST |
| Sequence 2362 | AI174957 | dbEST |
| Sequence 2363 | AI174991 | dbEST |
| Sequence 2364 | AI183358 | dbEST |
| Sequence 2365 | AI183447 | dbEST |
| Sequence 2366 | AI184562 | dbEST |
| Sequence 2367 | AI191244 | dbEST |
| Sequence 2368 | AI192767 | dbEST |
| Sequence 2369 | AI198311 | dbEST |
| Sequence 2370 | AI198986 | dbEST |
| Sequence 2371 | AI199094 | dbEST |
| Sequence 2372 | AI200038 | dbEST |
| Sequence 2373 | AI200110 | dbEST |
| Sequence 2374 | AI204090 | dbEST |
| Sequence 2375 | AI206199 | dbEST |
| Sequence 2376 | AI206344 | dbEST |
| Sequence 2377 | AI207367 | dbEST |
| Sequence 2378 | AI208947 | dbEST |
| Sequence 2379 | AI215841 | dbEST |
| Sequence 2380 | AI216969 | dbEST |
| Sequence 2381 | AI216988 | dbEST |
| Sequence 2382 | AI217003 | dbEST |
| Sequence 2383 | AI217012 | dbEST |
| Sequence 2384 | AI217021 | dbEST |
| Sequence 2385 | AI217023 | dbEST |
| Sequence 2386 | AI217425 | dbEST |
| Sequence 2387 | AI218376 | dbEST |
| Sequence 2388 | AI218882 | dbEST |
| Sequence 2389 | AI219324 | dbEST |
| Sequence 2390 | AI221829 | dbEST |
| Sequence 2391 | AI223085 | dbEST |
| Sequence 2392 | AI224519 | dbEST |
| Sequence 2393 | AI225043 | dbEST |
| Sequence 2394 | AI239822 | dbEST |
| Sequence 2395 | AI240872 | dbEST |
| Sequence 2396 | AI243423 | dbEST |
| Sequence 2397 | AI243780 | dbEST |
| Sequence 2398 | AI248277 | dbEST |
| Sequence 2399 | AI248736 | dbEST |
| Sequence 2400 | AI249000 | dbEST |
| Sequence 2401 | AI249797 | dbEST |
| Sequence 2402 | AI253330 | dbEST |
| Sequence 2403 | AI253335 | dbEST |
| Sequence 2404 | AI253338 | dbEST |
| Sequence 2405 | AI253379 | dbEST |
| Sequence 2406 | AI253436 | dbEST |
| Sequence 2407 | AI262486 | dbEST |
| Sequence 2408 | AI265776 | dbEST |
| Sequence 2409 | AI267162 | dbEST |
| Sequence 2410 | AI267185 | dbEST |
| Sequence 2411 | AI267276 | dbEST |
| Sequence 2412 | AI267282 | dbEST |
| Sequence 2413 | AI267307 | dbEST |
| Sequence 2414 | AI267454 | dbEST |
| Sequence 2415 | AI267502 | dbEST |
| Sequence 2416 | AI268328 | dbEST |
| Sequence 2417 | AI268405 | dbEST |
| Sequence 2418 | AI268860 | dbEST |
| Sequence 2419 | AI269060 | dbEST |
| Sequence 2420 | AI270511 | dbEST |
| Sequence 2421 | AI272941 | dbEST |
| Sequence 2422 | AI275175 | dbEST |
| Sequence 2423 | AI279838 | dbEST |
| Sequence 2424 | AI282277 | dbEST |
| Sequence 2425 | AI285488 | dbEST |
| Sequence 2426 | AI289220 | dbEST |
| Sequence 2427 | AI291247 | dbEST |
| Sequence 2428 | AI299524 | dbEST |
| Sequence 2429 | AI302137 | dbEST |
| Sequence 2430 | AI302799 | dbEST |
| Sequence 2431 | AI304947 | dbEST |
| Sequence 2432 | AI307402 | dbEST |
| Sequence 2433 | AI307515 | dbEST |
| Sequence 2434 | AI318280 | dbEST |
| Sequence 2435 | AI334833 | dbEST |
| Sequence 2436 | AI334964 | dbEST |
| Sequence 2437 | AI335153 | dbEST |
| Sequence 2438 | AI335554 | dbEST |
| Sequence 2439 | AI336147 | dbEST |
| Sequence 2440 | AI338335 | dbEST |
| Sequence 2441 | AI345416 | dbEST |
| Sequence 2442 | AI347506 | dbEST |
| Sequence 2443 | AI356718 | dbEST |
| Sequence 2444 | AI362403 | dbEST |
| Sequence 2445 | AI362462 | dbEST |
| Sequence 2446 | AI364053 | dbEST |
| Sequence 2447 | AI367350 | dbEST |
| Sequence 2448 | AI369174 | dbEST |
| Sequence 2449 | AI376513 | dbEST |
| Sequence 2450 | AI376865 | dbEST |
| Sequence 2451 | AI377194 | dbEST |
| Sequence 2452 | AI391631 | dbEST |
| Sequence 2453 | AI394646 | dbEST |
| Sequence 2454 | AI400752 | dbEST |
| Sequence 2455 | AI401840 | dbEST |
| Sequence 2456 | AI420227 | dbEST |
| Sequence 2457 | AI431814 | dbEST |
| Sequence 2458 | AI433818 | dbEST |
| Sequence 2459 | AI439774 | dbEST |
| Sequence 2460 | AI452703 | dbEST |
| Sequence 2461 | AI469038 | dbEST |
| Sequence 2462 | AI472525 | dbEST |
| Sequence 2463 | AI474466 | dbEST |
| Sequence 2464 | AI476318 | dbEST |
| Sequence 2465 | AI478365 | dbEST |
| Sequence 2466 | AI479439 | dbEST |
| Sequence 2467 | AI494299 | dbEST |
| Sequence 2468 | AI499393 | dbEST |
| Sequence 2469 | AI499986 | dbEST |
| Sequence 2470 | AI521100 | dbEST |
| Sequence 2471 | AI523506 | dbEST |
| Sequence 2472 | AI525231 | dbEST |
| Sequence 2473 | AI525454 | dbEST |
| Sequence 2474 | AI536024 | dbEST |
| Sequence 2475 | AI539349 | dbEST |
| Sequence 2476 | AI547146 | dbEST |
| Sequence 2477 | AI554016 | dbEST |
| Sequence 2478 | AI557112 | dbEST |
| Sequence 2479 | AI557116 | dbEST |
| Sequence 2480 | AI557182 | dbEST |
| Sequence 2481 | AI557225 | dbEST |
| Sequence 2482 | AI557231 | dbEST |
| Sequence 2483 | AI557235 | dbEST |
| Sequence 2484 | AI557246 | dbEST |
| Sequence 2485 | AI557495 | dbEST |
| Sequence 2486 | AI557599 | dbEST |
| Sequence 2487 | AI557626 | dbEST |
| Sequence 2488 | AI560958 | dbEST |
| Sequence 2489 | AI561122 | dbEST |
| Sequence 2490 | AI564475 | dbEST |
| Sequence 2491 | AI564487 | dbEST |
| Sequence 2492 | AI580160 | dbEST |
| Sequence 2493 | AI580162 | dbEST |
| Sequence 2494 | AI580927 | dbEST |
| Sequence 2495 | AI589476 | dbEST |
| Sequence 2496 | AI591088 | dbEST |
| Sequence 2497 | AI620673 | dbEST |
| Sequence 2498 | AI621153 | dbEST |
| Sequence 2499 | AI623315 | dbEST |
| Sequence 2500 | AI623804 | dbEST |
| Sequence 2501 | AI625404 | dbEST |
| Sequence 2502 | AI625747 | dbEST |
| Sequence 2503 | AI627356 | dbEST |
| Sequence 2504 | AI627542 | dbEST |
| Sequence 2505 | AI630104 | dbEST |
| Sequence 2506 | AI630188 | dbEST |
| Sequence 2507 | AI630634 | dbEST |
| Sequence 2508 | AI632523 | dbEST |
| Sequence 2509 | AI632534 | dbEST |
| Sequence 2510 | AI636943 | dbEST |
| Sequence 2511 | AI638414 | dbEST |
| Sequence 2512 | AI650721 | dbEST |
| Sequence 2513 | AI651195 | dbEST |
| Sequence 2514 | AI653319 | dbEST |
| Sequence 2515 | AI653621 | dbEST |
| Sequence 2516 | AI659898 | dbEST |
| Sequence 2517 | AI669210 | dbEST |
| Sequence 2518 | AI670009 | dbEST |
| Sequence 2519 | AI680343 | dbEST |
| Sequence 2520 | AI683431 | dbEST |
| Sequence 2521 | AI688098 | dbEST |
| Sequence 2522 | AI689722 | dbEST |
| Sequence 2523 | AI689762 | dbEST |
| Sequence 2524 | AI690157 | dbEST |
| Sequence 2525 | AI693561 | dbEST |
| Sequence 2526 | AI700642 | dbEST |
| Sequence 2527 | AI709356 | dbEST |
| Sequence 2528 | AI732680 | dbEST |
| Sequence 2529 | AI734209 | dbEST |
| Sequence 2530 | AI738605 | dbEST |
| Sequence 2531 | AI740900 | dbEST |
| Sequence 2532 | AI741486 | dbEST |
| Sequence 2533 | AI741633 | dbEST |
| Sequence 2534 | AI742115 | dbEST |
| Sequence 2535 | AI743303 | dbEST |
| Sequence 2536 | AI743852 | dbEST |
| Sequence 2537 | AI750682 | dbEST |
| Sequence 2538 | AI753280 | dbEST |
| Sequence 2539 | AI753951 | dbEST |
| Sequence 2540 | AI755085 | dbEST |
| Sequence 2541 | AI761291 | dbEST |
| Sequence 2542 | AI761469 | dbEST |
| Sequence 2543 | AI761986 | dbEST |
| Sequence 2544 | AI764962 | dbEST |
| Sequence 2545 | AI768624 | dbEST |
| Sequence 2546 | AI768854 | dbEST |
| Sequence 2547 | AI768877 | dbEST |
| Sequence 2548 | AI770098 | dbEST |
| Sequence 2549 | AI791179 | dbEST |
| Sequence 2550 | AI791419 | dbEST |
| Sequence 2551 | AI796120 | dbEST |
| Sequence 2552 | AI797042 | dbEST |
| Sequence 2553 | AI800250 | dbEST |
| Sequence 2554 | AI800673 | dbEST |
| Sequence 2555 | AI804662 | dbEST |
| Sequence 2556 | AI805522 | dbEST |
| Sequence 2557 | AI809314 | dbEST |
| Sequence 2558 | AI810002 | dbEST |
| Sequence 2559 | AI815499 | dbEST |
| Sequence 2560 | AI816322 | dbEST |
| Sequence 2561 | AI821437 | dbEST |
| Sequence 2562 | AI826105 | dbEST |
| Sequence 2563 | AI830758 | dbEST |
| Sequence 2564 | AI831462 | dbEST |
| Sequence 2565 | AI863042 | dbEST |
| Sequence 2566 | AI864870 | dbEST |
| Sequence 2567 | AI867463 | dbEST |
| Sequence 2568 | AI873501 | dbEST |
| Sequence 2569 | AI878926 | dbEST |
| Sequence 2570 | AI879040 | dbEST |
| Sequence 2571 | AI879248 | dbEST |
| Sequence 2572 | AI884963 | dbEST |
| Sequence 2573 | AI887875 | dbEST |
| Sequence 2574 | AI914133 | dbEST |
| Sequence 2575 | AI923011 | dbEST |
| Sequence 2576 | AI923978 | dbEST |
| Sequence 2577 | AI929113 | dbEST |
| Sequence 2578 | AI936548 | dbEST |
| Sequence 2579 | AI936562 | dbEST |
| Sequence 2580 | AI948503 | dbEST |
| Sequence 2581 | AI949422 | dbEST |
| Sequence 2582 | AI951118 | dbEST |
| Sequence 2583 | AI951970 | dbEST |
| Sequence 2584 | AI953711 | dbEST |
| Sequence 2585 | AI969567 | dbEST |
| Sequence 2586 | AI972279 | dbEST |
| Sequence 2587 | AI972479 | dbEST |
| Sequence 2588 | AI979288 | dbEST |
| Sequence 2589 | AI986240 | dbEST |
| Sequence 2590 | AL036499 | dbEST |
| Sequence 2591 | AL037031 | dbEST |
| Sequence 2592 | AL037226 | dbEST |
| Sequence 2593 | AL037707 | dbEST |
| Sequence 2594 | AL037798 | dbEST |
| Sequence 2595 | AL037828 | dbEST |
| Sequence 2596 | AL037847 | dbEST |
| Sequence 2597 | AL038802 | dbEST |
| Sequence 2598 | AL038985 | dbEST |
| Sequence 2599 | AL039254 | dbEST |
| Sequence 2600 | AL039293 | dbEST |
| Sequence 2601 | AL039542 | dbEST |
| Sequence 2602 | AL040692 | dbEST |
| Sequence 2603 | AL041050 | dbEST |
| Sequence 2604 | AL041176 | dbEST |
| Sequence 2605 | AL042234 | dbEST |
| Sequence 2606 | AL042613 | dbEST |
| Sequence 2607 | AL043034 | dbEST |
| Sequence 2608 | AL043506 | dbEST |
| Sequence 2609 | AL043864 | dbEST |
| Sequence 2610 | AL044891 | dbEST |
| Sequence 2611 | AL045213 | dbEST |
| Sequence 2612 | AL045331 | dbEST |
| Sequence 2613 | AL045538 | dbEST |
| Sequence 2614 | AL048890 | dbEST |
| Sequence 2615 | AL118999 | dbEST |
| Sequence 2616 | AL120381 | dbEST |
| Sequence 2617 | AW002185 | dbEST |
| Sequence 2618 | AW003247 | dbEST |
| Sequence 2619 | AW008473 | dbEST |
| Sequence 2620 | AW015055 | dbEST |
| Sequence 2621 | AW020699 | dbEST |
| Sequence 2622 | AW022599 | dbEST |
| Sequence 2623 | AW027339 | dbEST |
| Sequence 2624 | AW028594 | dbEST |
| Sequence 2625 | AW028606 | dbEST |
| Sequence 2626 | AW044547 | dbEST |
| Sequence 2627 | AW067913 | dbEST |
| Sequence 2628 | AW071696 | dbEST |
| Sequence 2629 | AW076087 | dbEST |
| Sequence 2630 | AW078747 | dbEST |
| Sequence 2631 | AW084017 | dbEST |
| Sequence 2632 | AW102951 | dbEST |
| Sequence 2633 | AW104561 | dbEST |
| Sequence 2634 | AW118457 | dbEST |
| Sequence 2635 | AW118595 | dbEST |
| Sequence 2636 | AW131770 | dbEST |
| Sequence 2637 | AW134772 | dbEST |
| Sequence 2638 | AW135359 | dbEST |
| Sequence 2639 | AW138731 | dbEST |
| Sequence 2640 | AW140056 | dbEST |
| Sequence 2641 | AW148884 | dbEST |
| Sequence 2642 | AW150276 | dbEST |
| Sequence 2643 | AW152311 | dbEST |
| Sequence 2644 | AW157060 | dbEST |
| Sequence 2645 | AW157303 | dbEST |
| Sequence 2646 | AW160447 | dbEST |
| Sequence 2647 | AW167579 | dbEST |
| Sequence 2648 | AW176926 | dbEST |
| Sequence 2649 | C15322 | dbEST |
| Sequence 2650 | C18232 | dbEST |
| Sequence 2651 | D50977 | dbEST |
| Sequence 2652 | D54335 | dbEST |
| Sequence 2653 | D58694 | dbEST |
| Sequence 2654 | D60970 | dbEST |
| Sequence 2655 | D81855 | dbEST |
| Sequence 2656 | F02595 | dbEST |
| Sequence 2657 | F08493 | dbEST |
| Sequence 2658 | F27229 | dbEST |
| Sequence 2659 | H03650 | dbEST |
| Sequence 2660 | H03785 | dbEST |
| Sequence 2661 | H06621 | dbEST |
| Sequence 2662 | H06944 | dbEST |
| Sequence 2663 | H09332 | dbEST |
| Sequence 2664 | H12519 | dbEST |
| Sequence 2665 | H24176 | dbEST |
| Sequence 2666 | H24856 | dbEST |
| Sequence 2667 | H26492 | dbEST |
| Sequence 2668 | H40738 | dbEST |
| Sequence 2669 | H47252 | dbEST |
| Sequence 2670 | H47363 | dbEST |
| Sequence 2671 | H49329 | dbEST |
| Sequence 2672 | H49828 | dbEST |
| Sequence 2673 | H57382 | dbEST |
| Sequence 2674 | H57485 | dbEST |
| Sequence 2675 | H60344 | dbEST |
| Sequence 2676 | H73387 | dbEST |
| Sequence 2677 | H82380 | dbEST |
| Sequence 2678 | H87420 | dbEST |
| Sequence 2679 | H93552 | dbEST |
| Sequence 2680 | H94179 | dbEST |
| Sequence 2681 | H95161 | dbEST |
| Sequence 2682 | N27899 | dbEST |
| Sequence 2683 | N32072 | dbEST |
| Sequence 2684 | N34186 | dbEST |
| Sequence 2685 | N44337 | dbEST |
| Sequence 2686 | N45167 | dbEST |
| Sequence 2687 | N46947 | dbEST |
| Sequence 2688 | N52271 | dbEST |
| Sequence 2689 | N52432 | dbEST |
| Sequence 2690 | N53033 | dbEST |
| Sequence 2691 | N54973 | dbEST |
| Sequence 2692 | N64576 | dbEST |
| Sequence 2693 | N72648 | dbEST |
| Sequence 2694 | N72922 | dbEST |
| Sequence 2695 | N74267 | dbEST |
| Sequence 2696 | N76672 | dbEST |
| Sequence 2697 | N79427 | dbEST |
| Sequence 2698 | N92160 | dbEST |
| Sequence 2699 | N94098 | dbEST |
| Sequence 2700 | R10438 | dbEST |
| Sequence 2701 | R10553 | dbEST |
| Sequence 2702 | R13850 | dbEST |
| Sequence 2703 | R24803 | dbEST |
| Sequence 2704 | R28584 | dbEST |
| Sequence 2705 | R34443 | dbEST |
| Sequence 2706 | R35797 | dbEST |
| Sequence 2707 | R45144 | dbEST |
| Sequence 2708 | R48890 | dbEST |
| Sequence 2709 | R70639 | dbEST |
| Sequence 2710 | R76235 | dbEST |
| Sequence 2711 | R76244 | dbEST |
| Sequence 2712 | R77186 | dbEST |
| Sequence 2713 | R80279 | dbEST |
| Sequence 2714 | R80852 | dbEST |
| Sequence 2715 | R84308 | dbEST |
| Sequence 2716 | R86265 | dbEST |
| Sequence 2717 | R87877 | dbEST |
| Sequence 2718 | R89611 | dbEST |
| Sequence 2719 | R92548 | dbEST |
| Sequence 2720 | R95821 | dbEST |
| Sequence 2721 | T10145 | dbEST |
| Sequence 2722 | T18977 | dbEST |
| Sequence 2723 | T32212 | dbEST |
| Sequence 2724 | T41232 | dbEST |
| Sequence 2725 | T52823 | dbEST |
| Sequence 2726 | T53424 | dbEST |
| Sequence 2727 | T54095 | dbEST |
| Sequence 2728 | T57386 | dbEST |
| Sequence 2729 | T64630 | dbEST |
| Sequence 2730 | T65098 | dbEST |
| Sequence 2731 | T67265 | dbEST |
| Sequence 2732 | T78615 | dbEST |
| Sequence 2733 | T83709 | dbEST |
| Sequence 2734 | T93613 | dbEST |
| Sequence 2735 | T99715 | dbEST |
| Sequence 2736 | U46309 | dbEST |
| Sequence 2737 | W04421 | dbEST |
| Sequence 2738 | W19519 | dbEST |
| Sequence 2739 | W25008 | dbEST |
| Sequence 2740 | W31992 | dbEST |
| Sequence 2741 | W39298 | dbEST |
| Sequence 2742 | W42653 | dbEST |
| Sequence 2743 | W52896 | dbEST |
| Sequence 2744 | W56077 | dbEST |
| Sequence 2745 | W63676 | dbEST |
| Sequence 2746 | W68028 | dbEST |
| Sequence 2747 | W86160 | dbEST |
| Sequence 2748 | W87865 | dbEST |
| Sequence 2749 | W88823 | dbEST |
| Sequence 2750 | W90224 | dbEST |
| Sequence 2751 | W91890 | dbEST |
| Sequence 2752 | Z43146 | dbEST |
| Sequence 2753 | Z43843 | dbEST |
| Sequence 2754 | Z45274 | dbEST |
| Sequence 2755 | Q44278 | NUCPATENT |
| Sequence 2756 | Q46540 | NUCPATENT |
| Sequence 2757 | Q49943 | NUCPATENT |
| Sequence 2758 | Q53478 | NUCPATENT |
| Sequence 2759 | T18813 | NUCPATENT |
| Sequence 2760 | T28638 | NUCPATENT |
| Sequence 2761 | T43474 | NUCPATENT |
| Sequence 2762 | T59274 | NUCPATENT |
| Sequence 2763 | V26460 | NUCPATENT |
| Sequence 2764 | V41451 | NUCPATENT |
| Sequence 2765 | V47539 | NUCPATENT |
| Sequence 2766 | V49578 | NUCPATENT |
| Sequence 2767 | V58523 | NUCPATENT |
| Sequence 2768 | V58544 | NUCPATENT |
| Sequence 2769 | V58628 | NUCPATENT |
| Sequence 2770 | V58685 | NUCPATENT |
| Sequence 2771 | V58761 | NUCPATENT |
| Sequence 2772 | V59638 | NUCPATENT |
| Sequence 2773 | V61487 | NUCPATENT |
| Sequence 2774 | V62427 | NUCPATENT |
| Sequence 2775 | V62428 | NUCPATENT |
| Sequence 2776 | V62430 | NUCPATENT |
| Sequence 2777 | V68992 | NUCPATENT |
| Sequence 2778 | V84497 | NUCPATENT |
| Sequence 2779 | V84510 | NUCPATENT |
| Sequence 2780 | V87984 | NUCPATENT |
| Sequence 2781 | V90138 | NUCPATENT |
| Sequence 2782 | X00630 | NUCPATENT |
| Sequence 2783 | X00632 | NUCPATENT |
| Sequence 2784 | X03841 | NUCPATENT |
| Sequence 2785 | X04407 | NUCPATENT |
| Sequence 2786 | X25445 | NUCPATENT |
| Sequence 2787 | X26850 | NUCPATENT |
| Sequence 2788 | X27262 | NUCPATENT |
| Sequence 2789 | X27278 | NUCPATENT |
| Sequence 2790 | X37486 | NUCPATENT |
| Sequence 2791 | X40477 | NUCPATENT |
| Sequence 2792 | X40518 | NUCPATENT |
| Sequence 2793 | X40711 | NUCPATENT |
| Sequence 2794 | X41185 | NUCPATENT |
| Sequence 2795 | X90444 | NUCPATENT |
| Sequence 2796 | X97772 | NUCPATENT |
| Sequence 2797 | X98891 | NUCPATENT |
| Sequence 2798 | Z11491 | NUCPATENT |
| Sequence 2799 | Z14468 | NUCPATENT |
| Sequence 2800 | Z15132 | NUCPATENT |
| Sequence 2801 | Z16412 | NUCPATENT |
| Sequence 2802 | AC28312 | PREPATNUC |
| Sequence 2803 | AC31081 | PREPATNUC |
| Sequence 2804 | AC31093 | PREPATNUC |
| Sequence 2805 | AC33927 | PREPATNUC |
| Sequence 2806 | A06846 | GENBANK |
| Sequence 2807 | A17546 | GENBANK |
| Sequence 2808 | A18657 | GENBANK |
| Sequence 2809 | AB000095 | GENBANK |
| Sequence 2810 | AB000468 | GENBANK |
| Sequence 2811 | AB002303 | GENBANK |
| Sequence 2812 | AB002311 | GENBANK |
| Sequence 2813 | AB002319 | GENBANK |
| Sequence 2814 | AB002330 | GENBANK |
| Sequence 2815 | AB002331 | GENBANK |
| Sequence 2816 | AB002365 | GENBANK |
| Sequence 2817 | AB002366 | GENBANK |
| Sequence 2818 | AB002370 | GENBANK |
| Sequence 2819 | AB002380 | GENBANK |
| Sequence 2820 | AB002386 | GENBANK |
| Sequence 2821 | AB004788 | GENBANK |
| Sequence 2822 | AB004851 | GENBANK |
| Sequence 2823 | AB006625 | GENBANK |
| Sequence 2824 | AB007891 | GENBANK |
| Sequence 2825 | AB007899 | GENBANK |
| Sequence 2826 | AB007916 | GENBANK |
| Sequence 2827 | AB007929 | GENBANK |
| Sequence 2828 | AB007932 | GENBANK |
| Sequence 2829 | AB007935 | GENBANK |
| Sequence 2830 | AB007956 | GENBANK |
| Sequence 2831 | AB007957 | GENBANK |
| Sequence 2832 | AB010427 | GENBANK |
| Sequence 2833 | AB011087 | GENBANK |
| Sequence 2834 | AB011103 | GENBANK |
| Sequence 2835 | AB011108 | GENBANK |
| Sequence 2836 | AB011147 | GENBANK |
| Sequence 2837 | AB011164 | GENBANK |
| Sequence 2838 | AB011169 | GENBANK |
| Sequence 2839 | AB011170 | GENBANK |
| Sequence 2840 | AB013918 | GENBANK |
| Sequence 2841 | AB014511 | GENBANK |
| Sequence 2842 | AB014533 | GENBANK |
| Sequence 2843 | AB014536 | GENBANK |
| Sequence 2844 | AB014542 | GENBANK |
| Sequence 2845 | AB014577 | GENBANK |
| Sequence 2846 | AB014888 | GENBANK |
| Sequence 2847 | AB015907 | GENBANK |
| Sequence 2848 | AB016533 | GENBANK |
| Sequence 2849 | AB017430 | GENBANK |
| Sequence 2850 | AB018274 | GENBANK |
| Sequence 2851 | AB018320 | GENBANK |
| Sequence 2852 | AB018324 | GENBANK |
| Sequence 2853 | AB018330 | GENBANK |
| Sequence 2854 | AB018334 | GENBANK |
| Sequence 2855 | AB018338 | GENBANK |
| Sequence 2856 | AB018353 | GENBANK |
| Sequence 2857 | AB019524 | GENBANK |
| Sequence 2858 | AB019564 | GENBANK |
| Sequence 2859 | AB019568 | GENBANK |
| Sequence 2860 | AB019987 | GENBANK |
| Sequence 2861 | AB020650 | GENBANK |
| Sequence 2862 | AB020673 | GENBANK |
| Sequence 2863 | AB020682 | GENBANK |
| Sequence 2864 | AB020686 | GENBANK |
| Sequence 2865 | AB020880 | GENBANK |
| Sequence 2866 | AB021663 | GENBANK |
| Sequence 2867 | AB023198 | GENBANK |
| Sequence 2868 | AB023205 | GENBANK |
| Sequence 2869 | AB023215 | GENBANK |
| Sequence 2870 | AB024704 | GENBANK |
| Sequence 2871 | AB027466 | GENBANK |
| Sequence 2872 | AB028624 | GENBANK |
| Sequence 2873 | AB028951 | GENBANK |
| Sequence 2874 | AB028995 | GENBANK |
| Sequence 2875 | AB029008 | GENBANK |
| Sequence 2876 | AB029025 | GENBANK |
| Sequence 2877 | AB029290 | GENBANK |
| Sequence 2878 | AB032969 | GENBANK |
| Sequence 2879 | AB032983 | GENBANK |
| Sequence 2880 | AB032994 | GENBANK |
| Sequence 2881 | AB033007 | GENBANK |
| Sequence 2882 | AB033010 | GENBANK |
| Sequence 2883 | AB033033 | GENBANK |
| Sequence 2884 | AB033059 | GENBANK |
| Sequence 2885 | AB033070 | GENBANK |
| Sequence 2886 | AB033091 | GENBANK |
| Sequence 2887 | AB033899 | GENBANK |
| Sequence 2888 | AF000152 | GENBANK |
| Sequence 2889 | AF000381 | GENBANK |
| Sequence 2890 | AF001893 | GENBANK |
| Sequence 2891 | AF002210 | GENBANK |
| Sequence 2892 | AF002668 | GENBANK |
| Sequence 2893 | AF003341 | GENBANK |
| Sequence 2894 | AF004561 | GENBANK |
| Sequence 2895 | AF004813 | GENBANK |
| Sequence 2896 | AF005038 | GENBANK |
| Sequence 2897 | AF005043 | GENBANK |
| Sequence 2898 | AF005050 | GENBANK |
| Sequence 2899 | AF006086 | GENBANK |
| Sequence 2900 | AF006514 | GENBANK |
| Sequence 2901 | AF007144 | GENBANK |
| Sequence 2902 | AF007151 | GENBANK |
| Sequence 2903 | AF007216 | GENBANK |
| Sequence 2904 | AF009620 | GENBANK |
| Sequence 2905 | AF010233 | GENBANK |
| Sequence 2906 | AF010313 | GENBANK |
| Sequence 2907 | AF011904 | GENBANK |
| Sequence 2908 | AF012072 | GENBANK |
| Sequence 2909 | AF013168 | GENBANK |
| Sequence 2910 | AF013759 | GENBANK |
| Sequence 2911 | AF013970 | GENBANK |
| Sequence 2912 | AF015283 | GENBANK |
| Sequence 2913 | AF020038 | GENBANK |
| Sequence 2914 | AF020202 | GENBANK |
| Sequence 2915 | AF021336 | GENBANK |
| Sequence 2916 | AF021351 | GENBANK |
| Sequence 2917 | AF023676 | GENBANK |
| Sequence 2918 | AF025684 | GENBANK |
| Sequence 2919 | AF026169 | GENBANK |
| Sequence 2920 | AF026247 | GENBANK |
| Sequence 2921 | AF028832 | GENBANK |
| Sequence 2922 | AF029890 | GENBANK |
| Sequence 2923 | AF030234 | GENBANK |
| Sequence 2924 | AF030310 | GENBANK |
| Sequence 2925 | AF033095 | GENBANK |
| Sequence 2926 | AF035268 | GENBANK |
| Sequence 2927 | AF038440 | GENBANK |
| Sequence 2928 | AF038952 | GENBANK |
| Sequence 2929 | AF038963 | GENBANK |
| Sequence 2930 | AF041259 | GENBANK |
| Sequence 2931 | AF042166 | GENBANK |
| Sequence 2932 | AF042385 | GENBANK |
| Sequence 2933 | AF044671 | GENBANK |
| Sequence 2934 | AF044956 | GENBANK |
| Sequence 2935 | AF046785 | GENBANK |
| Sequence 2936 | AF047020 | GENBANK |
| Sequence 2937 | AF047185 | GENBANK |
| Sequence 2938 | AF047599 | GENBANK |
| Sequence 2939 | AF052113 | GENBANK |
| Sequence 2940 | AF052164 | GENBANK |
| Sequence 2941 | AF053070 | GENBANK |
| Sequence 2942 | AF053470 | GENBANK |
| Sequence 2943 | AF054187 | GENBANK |
| Sequence 2944 | AF054838 | GENBANK |
| Sequence 2945 | AF054990 | GENBANK |
| Sequence 2946 | AF055008 | GENBANK |
| Sequence 2947 | AF055022 | GENBANK |
| Sequence 2948 | AF055025 | GENBANK |
| Sequence 2949 | AF055033 | GENBANK |
| Sequence 2950 | AF056087 | GENBANK |
| Sequence 2951 | AF057160 | GENBANK |
| Sequence 2952 | AF058319 | GENBANK |
| Sequence 2953 | AF060152 | GENBANK |
| Sequence 2954 | AF060515 | GENBANK |
| Sequence 2955 | AF061258 | GENBANK |
| Sequence 2956 | AF061938 | GENBANK |
| Sequence 2957 | AF063657 | GENBANK |
| Sequence 2958 | AF064084 | GENBANK |
| Sequence 2959 | AF069765 | GENBANK |
| Sequence 2960 | AF070523 | GENBANK |
| Sequence 2961 | AF070561 | GENBANK |
| Sequence 2962 | AF070650 | GENBANK |
| Sequence 2963 | AF070657 | GENBANK |
| Sequence 2964 | AF070665 | GENBANK |
| Sequence 2965 | AF071202 | GENBANK |
| Sequence 2966 | AF072810 | GENBANK |
| Sequence 2967 | AF073310 | GENBANK |
| Sequence 2968 | AF075587 | GENBANK |
| Sequence 2969 | AF076929 | GENBANK |
| Sequence 2970 | AF077030 | GENBANK |
| Sequence 2971 | AF077043 | GENBANK |
| Sequence 2972 | AF077202 | GENBANK |
| Sequence 2973 | AF077599 | GENBANK |
| Sequence 2974 | AF081192 | GENBANK |
| Sequence 2975 | AF081282 | GENBANK |
| Sequence 2976 | AF082868 | GENBANK |
| Sequence 2977 | AF083190 | GENBANK |
| Sequence 2978 | AF083395 | GENBANK |
| Sequence 2979 | AF085359 | GENBANK |
| Sequence 2980 | AF085362 | GENBANK |
| Sequence 2981 | AF085844 | GENBANK |
| Sequence 2982 | AF085893 | GENBANK |
| Sequence 2983 | AF085917 | GENBANK |
| Sequence 2984 | AF086183 | GENBANK |
| Sequence 2985 | AF087481 | GENBANK |
| Sequence 2986 | AF092038 | GENBANK |
| Sequence 2987 | AF092905 | GENBANK |
| Sequence 2988 | AF100741 | GENBANK |
| Sequence 2989 | AF100755 | GENBANK |
| Sequence 2990 | AF102265 | GENBANK |
| Sequence 2991 | AF102988 | GENBANK |
| Sequence 2992 | AF103801 | GENBANK |
| Sequence 2993 | AF103804 | GENBANK |
| Sequence 2994 | AF103907 | GENBANK |
| Sequence 2995 | AF104013 | GENBANK |
| Sequence 2996 | AF110647 | GENBANK |
| Sequence 2997 | AF117949 | GENBANK |
| Sequence 2998 | AF119121 | GENBANK |
| Sequence 2999 | AF126424 | GENBANK |
| Sequence 3000 | AF126782 | GENBANK |
| Sequence 3001 | AF128406 | GENBANK |
| Sequence 3002 | AF131774 | GENBANK |
| Sequence 3003 | AF131856 | GENBANK |
| Sequence 3004 | AF132000 | GENBANK |
| Sequence 3005 | AF132937 | GENBANK |
| Sequence 3006 | AF133845 | GENBANK |
| Sequence 3007 | AF136450 | GENBANK |
| Sequence 3008 | AF139461 | GENBANK |
| Sequence 3009 | AF143235 | GENBANK |
| Sequence 3010 | AF144755 | GENBANK |
| Sequence 3011 | AF145029 | GENBANK |
| Sequence 3012 | AF145316 | GENBANK |
| Sequence 3013 | AF147367 | GENBANK |
| Sequence 3014 | AF150087 | GENBANK |
| Sequence 3015 | AF151103 | GENBANK |
| Sequence 3016 | AF151840 | GENBANK |
| Sequence 3017 | AF151861 | GENBANK |
| Sequence 3018 | AF151870 | GENBANK |
| Sequence 3019 | AF151902 | GENBANK |
| Sequence 3020 | AF155100 | GENBANK |
| Sequence 3021 | AF155113 | GENBANK |
| Sequence 3022 | AF155330 | GENBANK |
| Sequence 3023 | AF155568 | GENBANK |
| Sequence 3024 | AF157028 | GENBANK |
| Sequence 3025 | AF159056 | GENBANK |
| Sequence 3026 | AF165281 | GENBANK |
| Sequence 3027 | AF168956 | GENBANK |
| Sequence 3028 | AF169481 | GENBANK |
| Sequence 3029 | AF174595 | GENBANK |
| Sequence 3030 | AF176574 | GENBANK |
| Sequence 3031 | AF176702 | GENBANK |
| Sequence 3032 | AF179274 | GENBANK |
| Sequence 3033 | AF188745 | GENBANK |
| Sequence 3034 | AF188746 | GENBANK |
| Sequence 3035 | AF190167 | GENBANK |
| Sequence 3036 | AF191020 | GENBANK |
| Sequence 3037 | AJ000041 | GENBANK |
| Sequence 3038 | AJ001189 | GENBANK |
| Sequence 3039 | AJ001838 | GENBANK |
| Sequence 3040 | AJ002744 | GENBANK |
| Sequence 3041 | AJ004913 | GENBANK |
| Sequence 3042 | AJ005821 | GENBANK |
| Sequence 3043 | AJ005893 | GENBANK |
| Sequence 3044 | AJ006470 | GENBANK |
| Sequence 3045 | AJ010953 | GENBANK |
| Sequence 3046 | AJ011497 | GENBANK |
| Sequence 3047 | AJ012499 | GENBANK |
| Sequence 3048 | AJ132694 | GENBANK |
| Sequence 3049 | AJ242829 | GENBANK |
| Sequence 3050 | AJ243874 | GENBANK |
| Sequence 3051 | AL049229 | GENBANK |
| Sequence 3052 | AL049784 | GENBANK |
| Sequence 3053 | AL049934 | GENBANK |
| Sequence 3054 | AL050037 | GENBANK |
| Sequence 3055 | AL050101 | GENBANK |
| Sequence 3056 | AL050136 | GENBANK |
| Sequence 3057 | AL050265 | GENBANK |
| Sequence 3058 | AL050274 | GENBANK |
| Sequence 3059 | AL050282 | GENBANK |
| Sequence 3060 | AL050373 | GENBANK |
| Sequence 3061 | AL079279 | GENBANK |
| Sequence 3062 | AL080135 | GENBANK |
| Sequence 3063 | AL080172 | GENBANK |
| Sequence 3064 | AL080192 | GENBANK |
| Sequence 3065 | AL080209 | GENBANK |
| Sequence 3066 | AL110185 | GENBANK |
| Sequence 3067 | AL110256 | GENBANK |
| Sequence 3068 | AL110297 | GENBANK |
| Sequence 3069 | AL117237 | GENBANK |
| Sequence 3070 | AL117458 | GENBANK |
| Sequence 3071 | AL117526 | GENBANK |
| Sequence 3072 | AL117536 | GENBANK |
| Sequence 3073 | AL117537 | GENBANK |
| Sequence 3074 | AL117619 | GENBANK |
| Sequence 3075 | AL117666 | GENBANK |
| Sequence 3076 | AL133059 | GENBANK |
| Sequence 3077 | AL133080 | GENBANK |
| Sequence 3078 | D13315 | GENBANK |
| Sequence 3079 | D13388 | GENBANK |
| Sequence 3080 | D13641 | GENBANK |
| Sequence 3081 | D13866 | GENBANK |
| Sequence 3082 | D14662 | GENBANK |
| Sequence 3083 | D16111 | GENBANK |
| Sequence 3084 | D16937 | GENBANK |
| Sequence 3085 | D17069 | GENBANK |
| Sequence 3086 | D17260 | GENBANK |
| Sequence 3087 | D17554 | GENBANK |
| Sequence 3088 | D21209 | GENBANK |
| Sequence 3089 | D21243 | GENBANK |
| Sequence 3090 | D21260 | GENBANK |
| Sequence 3091 | D23672 | GENBANK |
| Sequence 3092 | D25274 | GENBANK |
| Sequence 3093 | D25328 | GENBANK |
| Sequence 3094 | D25542 | GENBANK |
| Sequence 3095 | D26362 | GENBANK |
| Sequence 3096 | D26485 | GENBANK |
| Sequence 3097 | D26600 | GENBANK |
| Sequence 3098 | D28589 | GENBANK |
| Sequence 3099 | D29012 | GENBANK |
| Sequence 3100 | D29643 | GENBANK |
| Sequence 3101 | D31764 | GENBANK |
| Sequence 3102 | D31886 | GENBANK |
| Sequence 3103 | D38073 | GENBANK |
| Sequence 3104 | D38551 | GENBANK |
| Sequence 3105 | D38583 | GENBANK |
| Sequence 3106 | D42047 | GENBANK |
| Sequence 3107 | D42063 | GENBANK |
| Sequence 3108 | D43950 | GENBANK |
| Sequence 3109 | D45370 | GENBANK |
| Sequence 3110 | D49387 | GENBANK |
| Sequence 3111 | D50371 | GENBANK |
| Sequence 3112 | D50372 | GENBANK |
| Sequence 3113 | D55696 | GENBANK |
| Sequence 3114 | D63475 | GENBANK |
| Sequence 3115 | D63478 | GENBANK |
| Sequence 3116 | D63997 | GENBANK |
| Sequence 3117 | D67029 | GENBANK |
| Sequence 3118 | D79991 | GENBANK |
| Sequence 3119 | D79994 | GENBANK |
| Sequence 3120 | D83032 | GENBANK |
| Sequence 3121 | D83703 | GENBANK |
| Sequence 3122 | D84476 | GENBANK |
| Sequence 3123 | D86972 | GENBANK |
| Sequence 3124 | D86984 | GENBANK |
| Sequence 3125 | D87292 | GENBANK |
| Sequence 3126 | D87328 | GENBANK |
| Sequence 3127 | D87437 | GENBANK |
| Sequence 3128 | D87438 | GENBANK |
| Sequence 3129 | D87452 | GENBANK |
| Sequence 3130 | D87455 | GENBANK |
| Sequence 3131 | D87666 | GENBANK |
| Sequence 3132 | D87667 | GENBANK |
| Sequence 3133 | D87735 | GENBANK |
| Sequence 3134 | D87953 | GENBANK |
| Sequence 3135 | D89289 | GENBANK |
| Sequence 3136 | D89675 | GENBANK |
| Sequence 3137 | D90228 | GENBANK |
| Sequence 3138 | D90427 | GENBANK |
| Sequence 3139 | E01500 | GENBANK |
| Sequence 3140 | E02135 | GENBANK |
| Sequence 3141 | E02628 | GENBANK |
| Sequence 3142 | E06721 | GENBANK |
| Sequence 3143 | J00200 | GENBANK |
| Sequence 3144 | J02871 | GENBANK |
| Sequence 3145 | J03007 | GENBANK |
| Sequence 3146 | J03040 | GENBANK |
| Sequence 3147 | J03464 | GENBANK |
| Sequence 3148 | J03473 | GENBANK |
| Sequence 3149 | J03544 | GENBANK |
| Sequence 3150 | J03592 | GENBANK |
| Sequence 3151 | J03779 | GENBANK |
| Sequence 3152 | J03799 | GENBANK |
| Sequence 3153 | J03827 | GENBANK |
| Sequence 3154 | J04177 | GENBANK |
| Sequence 3155 | J04443 | GENBANK |
| Sequence 3156 | J04478 | GENBANK |
| Sequence 3157 | J04615 | GENBANK |
| Sequence 3158 | J04759 | GENBANK |
| Sequence 3159 | J05176 | GENBANK |
| Sequence 3160 | J05192 | GENBANK |
| Sequence 3161 | J05211 | GENBANK |
| Sequence 3162 | J05633 | GENBANK |
| Sequence 3163 | K00409 | GENBANK |
| Sequence 3164 | K01911 | GENBANK |
| Sequence 3165 | L01042 | GENBANK |
| Sequence 3166 | L05092 | GENBANK |
| Sequence 3167 | L07033 | GENBANK |
| Sequence 3168 | L08044 | GENBANK |
| Sequence 3169 | L10284 | GENBANK |
| Sequence 3170 | L11566 | GENBANK |
| Sequence 3171 | L12387 | GENBANK |
| Sequence 3172 | L13434 | GENBANK |
| Sequence 3173 | L13852 | GENBANK |
| Sequence 3174 | L15203 | GENBANK |
| Sequence 3175 | L19597 | GENBANK |
| Sequence 3176 | L19713 | GENBANK |
| Sequence 3177 | L20010 | GENBANK |
| Sequence 3178 | L20859 | GENBANK |
| Sequence 3179 | L20941 | GENBANK |
| Sequence 3180 | L22009 | GENBANK |
| Sequence 3181 | L25081 | GENBANK |
| Sequence 3182 | L25085 | GENBANK |
| Sequence 3183 | L25610 | GENBANK |
| Sequence 3184 | L28010 | GENBANK |
| Sequence 3185 | L29008 | GENBANK |
| Sequence 3186 | L34087 | GENBANK |
| Sequence 3187 | L34839 | GENBANK |
| Sequence 3188 | L38951 | GENBANK |
| Sequence 3189 | L40403 | GENBANK |
| Sequence 3190 | L41143 | GENBANK |
| Sequence 3191 | L42542 | GENBANK |
| Sequence 3192 | L47345 | GENBANK |
| Sequence 3193 | L49399 | GENBANK |
| Sequence 3194 | M10036 | GENBANK |
| Sequence 3195 | M10119 | GENBANK |
| Sequence 3196 | M11233 | GENBANK |
| Sequence 3197 | M11353 | GENBANK |
| Sequence 3198 | M11560 | GENBANK |
| Sequence 3199 | M12938 | GENBANK |
| Sequence 3200 | M13520 | GENBANK |
| Sequence 3201 | M13692 | GENBANK |
| Sequence 3202 | M15395 | GENBANK |
| Sequence 3203 | M15796 | GENBANK |
| Sequence 3204 | M15885 | GENBANK |
| Sequence 3205 | M15990 | GENBANK |
| Sequence 3206 | M16247 | GENBANK |
| Sequence 3207 | M16553 | GENBANK |
| Sequence 3208 | M17254 | GENBANK |
| Sequence 3209 | M17885 | GENBANK |
| Sequence 3210 | M17886 | GENBANK |
| Sequence 3211 | M20260 | GENBANK |
| Sequence 3212 | M21895 | GENBANK |
| Sequence 3213 | M21896 | GENBANK |
| Sequence 3214 | M22918 | GENBANK |
| Sequence 3215 | M22920 | GENBANK |
| Sequence 3216 | M23613 | GENBANK |
| Sequence 3217 | M24194 | GENBANK |
| Sequence 3218 | M24594 | GENBANK |
| Sequence 3219 | M26663 | GENBANK |
| Sequence 3220 | M28016 | GENBANK |
| Sequence 3221 | M28211 | GENBANK |
| Sequence 3222 | M29366 | GENBANK |
| Sequence 3223 | M29536 | GENBANK |
| Sequence 3224 | M30448 | GENBANK |
| Sequence 3225 | M30818 | GENBANK |
| Sequence 3226 | M31899 | GENBANK |
| Sequence 3227 | M34840 | GENBANK |
| Sequence 3228 | M35252 | GENBANK |
| Sequence 3229 | M36072 | GENBANK |
| Sequence 3230 | M36647 | GENBANK |
| Sequence 3231 | M38690 | GENBANK |
| Sequence 3232 | M55409 | GENBANK |
| Sequence 3233 | M59849 | GENBANK |
| Sequence 3234 | M60457 | GENBANK |
| Sequence 3235 | M60828 | GENBANK |
| Sequence 3236 | M61831 | GENBANK |
| Sequence 3237 | M61866 | GENBANK |
| Sequence 3238 | M61916 | GENBANK |
| Sequence 3239 | M63573 | GENBANK |
| Sequence 3240 | M64098 | GENBANK |
| Sequence 3241 | M64241 | GENBANK |
| Sequence 3242 | M64572 | GENBANK |
| Sequence 3243 | M68840 | GENBANK |
| Sequence 3244 | M68864 | GENBANK |
| Sequence 3245 | M69177 | GENBANK |
| Sequence 3246 | M69181 | GENBANK |
| Sequence 3247 | M73547 | GENBANK |
| Sequence 3248 | M74509 | GENBANK |
| Sequence 3249 | M77142 | GENBANK |
| Sequence 3250 | M77830 | GENBANK |
| Sequence 3251 | M80783 | GENBANK |
| Sequence 3252 | M81757 | GENBANK |
| Sequence 3253 | M83205 | GENBANK |
| Sequence 3254 | M84711 | GENBANK |
| Sequence 3255 | M90054 | GENBANK |
| Sequence 3256 | M90309 | GENBANK |
| Sequence 3257 | M94345 | GENBANK |
| Sequence 3258 | M95627 | GENBANK |
| Sequence 3259 | S42658 | GENBANK |
| Sequence 3260 | S48196 | GENBANK |
| Sequence 3261 | S50015 | GENBANK |
| Sequence 3262 | S63912 | GENBANK |
| Sequence 3263 | S69272 | GENBANK |
| Sequence 3264 | S74678 | GENBANK |
| Sequence 3265 | S75755 | GENBANK |
| Sequence 3266 | S76756 | GENBANK |
| Sequence 3267 | S78203 | GENBANK |
| Sequence 3268 | U00930 | GENBANK |
| Sequence 3269 | U01184 | GENBANK |
| Sequence 3270 | U03688 | GENBANK |
| Sequence 3271 | U04209 | GENBANK |
| Sequence 3272 | U08815 | GENBANK |
| Sequence 3273 | U09367 | GENBANK |
| Sequence 3274 | U09410 | GENBANK |
| Sequence 3275 | U10248 | GENBANK |
| Sequence 3276 | U12465 | GENBANK |
| Sequence 3277 | U14603 | GENBANK |
| Sequence 3278 | U14968 | GENBANK |
| Sequence 3279 | U14969 | GENBANK |
| Sequence 3280 | U14970 | GENBANK |
| Sequence 3281 | U17077 | GENBANK |
| Sequence 3282 | U17104 | GENBANK |
| Sequence 3283 | U17105 | GENBANK |
| Sequence 3284 | U17838 | GENBANK |
| Sequence 3285 | U18197 | GENBANK |
| Sequence 3286 | U20770 | GENBANK |
| Sequence 3287 | U21090 | GENBANK |
| Sequence 3288 | U22897 | GENBANK |
| Sequence 3289 | U25766 | GENBANK |
| Sequence 3290 | U29091 | GENBANK |
| Sequence 3291 | U30897 | GENBANK |
| Sequence 3292 | U33760 | GENBANK |
| Sequence 3293 | U34038 | GENBANK |
| Sequence 3294 | U34994 | GENBANK |
| Sequence 3295 | U37230 | GENBANK |
| Sequence 3296 | U37518 | GENBANK |
| Sequence 3297 | U37558 | GENBANK |
| Sequence 3298 | U37690 | GENBANK |
| Sequence 3299 | U39840 | GENBANK |
| Sequence 3300 | U39945 | GENBANK |
| Sequence 3301 | U40272 | GENBANK |
| Sequence 3302 | U40572 | GENBANK |
| Sequence 3303 | U43701 | GENBANK |
| Sequence 3304 | U44839 | GENBANK |
| Sequence 3305 | U46751 | GENBANK |
| Sequence 3306 | U47674 | GENBANK |
| Sequence 3307 | U47742 | GENBANK |
| Sequence 3308 | U49957 | GENBANK |
| Sequence 3309 | U51134 | GENBANK |
| Sequence 3310 | U51678 | GENBANK |
| Sequence 3311 | U52969 | GENBANK |
| Sequence 3312 | U56255 | GENBANK |
| Sequence 3313 | U60116 | GENBANK |
| Sequence 3314 | U63289 | GENBANK |
| Sequence 3315 | U64315 | GENBANK |
| Sequence 3316 | U65785 | GENBANK |
| Sequence 3317 | U66197 | GENBANK |
| Sequence 3318 | U66469 | GENBANK |
| Sequence 3319 | U67171 | GENBANK |
| Sequence 3320 | U68140 | GENBANK |
| Sequence 3321 | U70323 | GENBANK |
| Sequence 3322 | U71363 | GENBANK |
| Sequence 3323 | U73824 | GENBANK |
| Sequence 3324 | U75272 | GENBANK |
| Sequence 3325 | U75329 | GENBANK |
| Sequence 3326 | U77720 | GENBANK |
| Sequence 3327 | U80040 | GENBANK |
| Sequence 3328 | U80213 | GENBANK |
| Sequence 3329 | U82226 | GENBANK |
| Sequence 3330 | U82756 | GENBANK |
| Sequence 3331 | U83115 | GENBANK |
| Sequence 3332 | U89505 | GENBANK |
| Sequence 3333 | U90907 | GENBANK |
| Sequence 3334 | U90942 | GENBANK |
| Sequence 3335 | U92458 | GENBANK |
| Sequence 3336 | U95825 | GENBANK |
| Sequence 3337 | X01037 | GENBANK |
| Sequence 3338 | X01742 | GENBANK |
| Sequence 3339 | X02530 | GENBANK |
| Sequence 3340 | X02544 | GENBANK |
| Sequence 3341 | X02596 | GENBANK |
| Sequence 3342 | X04098 | GENBANK |
| Sequence 3343 | X05332 | GENBANK |
| Sequence 3344 | X06272 | GENBANK |
| Sequence 3345 | X06747 | GENBANK |
| Sequence 3346 | X12451 | GENBANK |
| Sequence 3347 | X12654 | GENBANK |
| Sequence 3348 | X13238 | GENBANK |
| Sequence 3349 | X13425 | GENBANK |
| Sequence 3350 | X14420 | GENBANK |
| Sequence 3351 | X15653 | GENBANK |
| Sequence 3352 | X15729 | GENBANK |
| Sequence 3353 | X15822 | GENBANK |
| Sequence 3354 | X15949 | GENBANK |
| Sequence 3355 | X16940 | GENBANK |
| Sequence 3356 | X17206 | GENBANK |
| Sequence 3357 | X54941 | GENBANK |
| Sequence 3358 | X56807 | GENBANK |
| Sequence 3359 | X56932 | GENBANK |
| Sequence 3360 | X57398 | GENBANK |
| Sequence 3361 | X57500 | GENBANK |
| Sequence 3362 | X58141 | GENBANK |
| Sequence 3363 | X60111 | GENBANK |
| Sequence 3364 | X63422 | GENBANK |
| Sequence 3365 | X63432 | GENBANK |
| Sequence 3366 | X64707 | GENBANK |
| Sequence 3367 | X65923 | GENBANK |
| Sequence 3368 | X68277 | GENBANK |
| Sequence 3369 | X68560 | GENBANK |
| Sequence 3370 | X68684 | GENBANK |
| Sequence 3371 | X69086 | GENBANK |
| Sequence 3372 | X69111 | GENBANK |
| Sequence 3373 | X71129 | GENBANK |
| Sequence 3374 | X72727 | GENBANK |
| Sequence 3375 | X73114 | GENBANK |
| Sequence 3376 | X73608 | GENBANK |
| Sequence 3377 | X74801 | GENBANK |
| Sequence 3378 | X75304 | GENBANK |
| Sequence 3379 | X75535 | GENBANK |
| Sequence 3380 | X77753 | GENBANK |
| Sequence 3381 | X78136 | GENBANK |
| Sequence 3382 | X81889 | GENBANK |
| Sequence 3383 | X85134 | GENBANK |
| Sequence 3384 | X87241 | GENBANK |
| Sequence 3385 | X87949 | GENBANK |
| Sequence 3386 | X92689 | GENBANK |
| Sequence 3387 | X94754 | GENBANK |
| Sequence 3388 | X97335 | GENBANK |
| Sequence 3389 | Y00052 | GENBANK |
| Sequence 3390 | Y00815 | GENBANK |
| Sequence 3391 | Y09188 | GENBANK |
| Sequence 3392 | Y11164 | GENBANK |
| Sequence 3393 | Y12781 | GENBANK |
| Sequence 3394 | Y15286 | GENBANK |
| Sequence 3395 | Z22968 | GENBANK |
| Sequence 3396 | Z36715 | GENBANK |
| Sequence 3397 | Z47087 | GENBANK |
| Sequence 3398 | Z50749 | GENBANK |
| Sequence 3399 | AA001999 | dbEST |
| Sequence 3400 | AA004255 | dbEST |
| Sequence 3401 | AA004869 | dbEST |
| Sequence 3402 | AA007389 | dbEST |
| Sequence 3403 | AA007474 | dbEST |
| Sequence 3404 | AA009527 | dbEST |
| Sequence 3405 | AA010167 | dbEST |
| Sequence 3406 | AA010284 | dbEST |
| Sequence 3407 | AA011297 | dbEST |
| Sequence 3408 | AA011656 | dbEST |
| Sequence 3409 | AA011707 | dbEST |
| Sequence 3410 | AA013018 | dbEST |
| Sequence 3411 | AA017512 | dbEST |
| Sequence 3412 | AA017614 | dbEST |
| Sequence 3413 | AA018102 | dbEST |
| Sequence 3414 | AA018202 | dbEST |
| Sequence 3415 | AA018345 | dbEST |
| Sequence 3416 | AA018888 | dbEST |
| Sequence 3417 | AA019538 | dbEST |
| Sequence 3418 | AA021544 | dbEST |
| Sequence 3419 | AA022965 | dbEST |
| Sequence 3420 | AA025287 | dbEST |
| Sequence 3421 | AA028103 | dbEST |
| Sequence 3422 | AA028145 | dbEST |
| Sequence 3423 | AA029143 | dbEST |
| Sequence 3424 | AA031367 | dbEST |
| Sequence 3425 | AA032194 | dbEST |
| Sequence 3426 | AA033869 | dbEST |
| Sequence 3427 | AA034094 | dbEST |
| Sequence 3428 | AA035549 | dbEST |
| Sequence 3429 | AA035604 | dbEST |
| Sequence 3430 | AA035667 | dbEST |
| Sequence 3431 | AA035773 | dbEST |
| Sequence 3432 | AA037138 | dbEST |
| Sequence 3433 | AA037143 | dbEST |
| Sequence 3434 | AA041205 | dbEST |
| Sequence 3435 | AA041250 | dbEST |
| Sequence 3436 | AA043141 | dbEST |
| Sequence 3437 | AA044652 | dbEST |
| Sequence 3438 | AA044786 | dbEST |
| Sequence 3439 | AA045240 | dbEST |
| Sequence 3440 | AA045606 | dbEST |
| Sequence 3441 | AA045992 | dbEST |
| Sequence 3442 | AA047482 | dbEST |
| Sequence 3443 | AA047618 | dbEST |
| Sequence 3444 | AA053192 | dbEST |
| Sequence 3445 | AA053459 | dbEST |
| Sequence 3446 | AA054778 | dbEST |
| Sequence 3447 | AA055285 | dbEST |
| Sequence 3448 | AA055326 | dbEST |
| Sequence 3449 | AA055475 | dbEST |
| Sequence 3450 | AA055650 | dbEST |
| Sequence 3451 | AA056025 | dbEST |
| Sequence 3452 | AA056369 | dbEST |
| Sequence 3453 | AA057721 | dbEST |
| Sequence 3454 | AA058458 | dbEST |
| Sequence 3455 | AA058488 | dbEST |
| Sequence 3456 | AA058899 | dbEST |
| Sequence 3457 | AA063476 | dbEST |
| Sequence 3458 | AA063616 | dbEST |
| Sequence 3459 | AA063629 | dbEST |
| Sequence 3460 | AA064947 | dbEST |
| Sequence 3461 | AA065100 | dbEST |
| Sequence 3462 | AA065216 | dbEST |
| Sequence 3463 | AA069367 | dbEST |
| Sequence 3464 | AA069801 | dbEST |
| Sequence 3465 | AA069811 | dbEST |
| Sequence 3466 | AA069850 | dbEST |
| Sequence 3467 | AA070086 | dbEST |
| Sequence 3468 | AA074200 | dbEST |
| Sequence 3469 | AA074837 | dbEST |
| Sequence 3470 | AA075415 | dbEST |
| Sequence 3471 | AA075922 | dbEST |
| Sequence 3472 | AA076260 | dbEST |
| Sequence 3473 | AA076430 | dbEST |
| Sequence 3474 | AA076504 | dbEST |
| Sequence 3475 | AA078872 | dbEST |
| Sequence 3476 | AA079366 | dbEST |
| Sequence 3477 | AA079869 | dbEST |
| Sequence 3478 | AA080889 | dbEST |
| Sequence 3479 | AA082281 | dbEST |
| Sequence 3480 | AA082812 | dbEST |
| Sequence 3481 | AA082829 | dbEST |
| Sequence 3482 | AA083148 | dbEST |
| Sequence 3483 | AA083472 | dbEST |
| Sequence 3484 | AA083573 | dbEST |
| Sequence 3485 | AA083654 | dbEST |
| Sequence 3486 | AA083941 | dbEST |
| Sequence 3487 | AA085810 | dbEST |
| Sequence 3488 | AA086007 | dbEST |
| Sequence 3489 | AA086087 | dbEST |
| Sequence 3490 | AA088655 | dbEST |
| Sequence 3491 | AA088758 | dbEST |
| Sequence 3492 | AA088914 | dbEST |
| Sequence 3493 | AA089566 | dbEST |
| Sequence 3494 | AA090023 | dbEST |
| Sequence 3495 | AA090106 | dbEST |
| Sequence 3496 | AA090625 | dbEST |
| Sequence 3497 | AA091245 | dbEST |
| Sequence 3498 | AA092596 | dbEST |
| Sequence 3499 | AA092811 | dbEST |
| Sequence 3500 | AA093359 | dbEST |
| Sequence 3501 | AA095002 | dbEST |
| Sequence 3502 | AA096320 | dbEST |
| Sequence 3503 | AA096453 | dbEST |
| Sequence 3504 | AA098824 | dbEST |
| Sequence 3505 | AA099424 | dbEST |
| Sequence 3506 | AA099718 | dbEST |
| Sequence 3507 | AA100133 | dbEST |
| Sequence 3508 | AA101276 | dbEST |
| Sequence 3509 | AA101476 | dbEST |
| Sequence 3510 | AA102159 | dbEST |
| Sequence 3511 | AA111892 | dbEST |
| Sequence 3512 | AA112162 | dbEST |
| Sequence 3513 | AA113403 | dbEST |
| Sequence 3514 | AA113952 | dbEST |
| Sequence 3515 | AA114003 | dbEST |
| Sequence 3516 | AA114061 | dbEST |
| Sequence 3517 | AA115215 | dbEST |
| Sequence 3518 | AA120849 | dbEST |
| Sequence 3519 | AA121941 | dbEST |
| Sequence 3520 | AA125961 | dbEST |
| Sequence 3521 | AA126621 | dbEST |
| Sequence 3522 | AA126666 | dbEST |
| Sequence 3523 | AA126754 | dbEST |
| Sequence 3524 | AA127185 | dbEST |
| Sequence 3525 | AA127436 | dbEST |
| Sequence 3526 | AA127552 | dbEST |
| Sequence 3527 | AA128536 | dbEST |
| Sequence 3528 | AA128768 | dbEST |
| Sequence 3529 | AA130428 | dbEST |
| Sequence 3530 | AA130615 | dbEST |
| Sequence 3531 | AA131077 | dbEST |
| Sequence 3532 | AA131227 | dbEST |
| Sequence 3533 | AA131233 | dbEST |
| Sequence 3534 | AA132802 | dbEST |
| Sequence 3535 | AA132857 | dbEST |
| Sequence 3536 | AA132862 | dbEST |
| Sequence 3537 | AA133071 | dbEST |
| Sequence 3538 | AA133248 | dbEST |
| Sequence 3539 | AA133698 | dbEST |
| Sequence 3540 | AA133724 | dbEST |
| Sequence 3541 | AA133982 | dbEST |
| Sequence 3542 | AA134031 | dbEST |
| Sequence 3543 | AA134321 | dbEST |
| Sequence 3544 | AA134377 | dbEST |
| Sequence 3545 | AA135384 | dbEST |
| Sequence 3546 | AA135454 | dbEST |
| Sequence 3547 | AA135911 | dbEST |
| Sequence 3548 | AA136163 | dbEST |
| Sequence 3549 | AA143604 | dbEST |
| Sequence 3550 | AA146916 | dbEST |
| Sequence 3551 | AA147403 | dbEST |
| Sequence 3552 | AA147898 | dbEST |
| Sequence 3553 | AA147902 | dbEST |
| Sequence 3554 | AA148250 | dbEST |
| Sequence 3555 | AA148822 | dbEST |
| Sequence 3556 | AA150823 | dbEST |
| Sequence 3557 | AA150891 | dbEST |
| Sequence 3558 | AA151018 | dbEST |
| Sequence 3559 | AA151163 | dbEST |
| Sequence 3560 | AA151401 | dbEST |
| Sequence 3561 | AA151830 | dbEST |
| Sequence 3562 | AA151979 | dbEST |
| Sequence 3563 | AA151988 | dbEST |
| Sequence 3564 | AA152169 | dbEST |
| Sequence 3565 | AA156886 | dbEST |
| Sequence 3566 | AA157163 | dbEST |
| Sequence 3567 | AA157466 | dbEST |
| Sequence 3568 | AA157632 | dbEST |
| Sequence 3569 | AA157652 | dbEST |
| Sequence 3570 | AA158098 | dbEST |
| Sequence 3571 | AA158922 | dbEST |
| Sequence 3572 | AA159656 | dbEST |
| Sequence 3573 | AA159792 | dbEST |
| Sequence 3574 | AA159910 | dbEST |
| Sequence 3575 | AA160630 | dbEST |
| Sequence 3576 | AA164677 | dbEST |
| Sequence 3577 | AA165148 | dbEST |
| Sequence 3578 | AA167252 | dbEST |
| Sequence 3579 | AA171679 | dbEST |
| Sequence 3580 | AA171830 | dbEST |
| Sequence 3581 | AA173331 | dbEST |
| Sequence 3582 | AA173460 | dbEST |
| Sequence 3583 | AA176178 | dbEST |
| Sequence 3584 | AA179098 | dbEST |
| Sequence 3585 | AA179187 | dbEST |
| Sequence 3586 | AA179362 | dbEST |
| Sequence 3587 | AA179567 | dbEST |
| Sequence 3588 | AA179691 | dbEST |
| Sequence 3589 | AA180398 | dbEST |
| Sequence 3590 | AA180914 | dbEST |
| Sequence 3591 | AA181580 | dbEST |
| Sequence 3592 | AA181855 | dbEST |
| Sequence 3593 | AA182804 | dbEST |
| Sequence 3594 | AA182839 | dbEST |
| Sequence 3595 | AA186608 | dbEST |
| Sequence 3596 | AA187629 | dbEST |
| Sequence 3597 | AA188045 | dbEST |
| Sequence 3598 | AA188065 | dbEST |
| Sequence 3599 | AA188101 | dbEST |
| Sequence 3600 | AA188780 | dbEST |
| Sequence 3601 | AA188867 | dbEST |
| Sequence 3602 | AA190779 | dbEST |
| Sequence 3603 | AA190890 | dbEST |
| Sequence 3604 | AA191092 | dbEST |
| Sequence 3605 | AA191719 | dbEST |
| Sequence 3606 | AA192604 | dbEST |
| Sequence 3607 | AA193455 | dbEST |
| Sequence 3608 | AA194374 | dbEST |
| Sequence 3609 | AA194577 | dbEST |
| Sequence 3610 | AA194754 | dbEST |
| Sequence 3611 | AA195377 | dbEST |
| Sequence 3612 | AA203110 | dbEST |
| Sequence 3613 | AA203172 | dbEST |
| Sequence 3614 | AA205928 | dbEST |
| Sequence 3615 | AA206194 | dbEST |
| Sequence 3616 | AA206540 | dbEST |
| Sequence 3617 | AA206546 | dbEST |
| Sequence 3618 | AA206991 | dbEST |
| Sequence 3619 | AA209498 | dbEST |
| Sequence 3620 | AA210898 | dbEST |
| Sequence 3621 | AA216391 | dbEST |
| Sequence 3622 | AA218536 | dbEST |
| Sequence 3623 | AA218693 | dbEST |
| Sequence 3624 | AA219116 | dbEST |
| Sequence 3625 | AA219264 | dbEST |
| Sequence 3626 | AA219458 | dbEST |
| Sequence 3627 | AA220219 | dbEST |
| Sequence 3628 | AA220970 | dbEST |
| Sequence 3629 | AA223564 | dbEST |
| Sequence 3630 | AA224528 | dbEST |
| Sequence 3631 | AA224548 | dbEST |
| Sequence 3632 | AA224784 | dbEST |
| Sequence 3633 | AA225294 | dbEST |
| Sequence 3634 | AA225515 | dbEST |
| Sequence 3635 | AA226012 | dbEST |
| Sequence 3636 | AA226101 | dbEST |
| Sequence 3637 | AA226171 | dbEST |
| Sequence 3638 | AA226430 | dbEST |
| Sequence 3639 | AA227059 | dbEST |
| Sequence 3640 | AA227549 | dbEST |
| Sequence 3641 | AA228012 | dbEST |
| Sequence 3642 | AA229495 | dbEST |
| Sequence 3643 | AA232186 | dbEST |
| Sequence 3644 | AA233021 | dbEST |
| Sequence 3645 | AA233233 | dbEST |
| Sequence 3646 | AA233687 | dbEST |
| Sequence 3647 | AA234300 | dbEST |
| Sequence 3648 | AA234464 | dbEST |
| Sequence 3649 | AA234533 | dbEST |
| Sequence 3650 | AA234698 | dbEST |
| Sequence 3651 | AA235914 | dbEST |
| Sequence 3652 | AA236865 | dbEST |
| Sequence 3653 | AA242891 | dbEST |
| Sequence 3654 | AA242951 | dbEST |
| Sequence 3655 | AA244003 | dbEST |
| Sequence 3656 | AA244099 | dbEST |
| Sequence 3657 | AA249118 | dbEST |
| Sequence 3658 | AA250903 | dbEST |
| Sequence 3659 | AA251201 | dbEST |
| Sequence 3660 | AA251262 | dbEST |
| Sequence 3661 | AA255576 | dbEST |
| Sequence 3662 | AA256248 | dbEST |
| Sequence 3663 | AA256403 | dbEST |
| Sequence 3664 | AA256724 | dbEST |
| Sequence 3665 | AA258750 | dbEST |
| Sequence 3666 | AA259025 | dbEST |
| Sequence 3667 | AA262494 | dbEST |
| Sequence 3668 | AA263002 | dbEST |
| Sequence 3669 | AA263040 | dbEST |
| Sequence 3670 | AA263145 | dbEST |
| Sequence 3671 | AA278642 | dbEST |
| Sequence 3672 | AA278729 | dbEST |
| Sequence 3673 | AA280080 | dbEST |
| Sequence 3674 | AA280089 | dbEST |
| Sequence 3675 | AA280876 | dbEST |
| Sequence 3676 | AA282734 | dbEST |
| Sequence 3677 | AA283746 | dbEST |
| Sequence 3678 | AA284642 | dbEST |
| Sequence 3679 | AA284806 | dbEST |
| Sequence 3680 | AA286754 | dbEST |
| Sequence 3681 | AA286841 | dbEST |
| Sequence 3682 | AA287561 | dbEST |
| Sequence 3683 | AA287589 | dbEST |
| Sequence 3684 | AA291220 | dbEST |
| Sequence 3685 | AA291788 | dbEST |
| Sequence 3686 | AA292179 | dbEST |
| Sequence 3687 | AA292739 | dbEST |
| Sequence 3688 | AA292796 | dbEST |
| Sequence 3689 | AA293036 | dbEST |
| Sequence 3690 | AA293047 | dbEST |
| Sequence 3691 | AA293050 | dbEST |
| Sequence 3692 | AA296186 | dbEST |
| Sequence 3693 | AA296326 | dbEST |
| Sequence 3694 | AA296502 | dbEST |
| Sequence 3695 | AA296812 | dbEST |
| Sequence 3696 | AA296846 | dbEST |
| Sequence 3697 | AA296970 | dbEST |
| Sequence 3698 | AA297270 | dbEST |
| Sequence 3699 | AA297402 | dbEST |
| Sequence 3700 | AA298199 | dbEST |
| Sequence 3701 | AA298732 | dbEST |
| Sequence 3702 | AA298986 | dbEST |
| Sequence 3703 | AA299541 | dbEST |
| Sequence 3704 | AA299961 | dbEST |
| Sequence 3705 | AA300065 | dbEST |
| Sequence 3706 | AA300262 | dbEST |
| Sequence 3707 | AA301753 | dbEST |
| Sequence 3708 | AA301983 | dbEST |
| Sequence 3709 | AA302919 | dbEST |
| Sequence 3710 | AA302986 | dbEST |
| Sequence 3711 | AA303184 | dbEST |
| Sequence 3712 | AA303334 | dbEST |
| Sequence 3713 | AA303594 | dbEST |
| Sequence 3714 | AA303657 | dbEST |
| Sequence 3715 | AA303696 | dbEST |
| Sequence 3716 | AA304025 | dbEST |
| Sequence 3717 | AA305121 | dbEST |
| Sequence 3718 | AA305231 | dbEST |
| Sequence 3719 | AA305315 | dbEST |
| Sequence 3720 | AA305333 | dbEST |
| Sequence 3721 | AA305566 | dbEST |
| Sequence 3722 | AA305895 | dbEST |
| Sequence 3723 | AA305951 | dbEST |
| Sequence 3724 | AA306028 | dbEST |
| Sequence 3725 | AA306129 | dbEST |
| Sequence 3726 | AA306402 | dbEST |
| Sequence 3727 | AA306668 | dbEST |
| Sequence 3728 | AA306983 | dbEST |
| Sequence 3729 | AA307101 | dbEST |
| Sequence 3730 | AA307579 | dbEST |
| Sequence 3731 | AA307590 | dbEST |
| Sequence 3732 | AA307728 | dbEST |
| Sequence 3733 | AA307746 | dbEST |
| Sequence 3734 | AA307789 | dbEST |
| Sequence 3735 | AA307941 | dbEST |
| Sequence 3736 | AA308063 | dbEST |
| Sequence 3737 | AA308269 | dbEST |
| Sequence 3738 | AA308533 | dbEST |
| Sequence 3739 | AA308942 | dbEST |
| Sequence 3740 | AA308978 | dbEST |
| Sequence 3741 | AA309445 | dbEST |
| Sequence 3742 | AA309812 | dbEST |
| Sequence 3743 | AA310163 | dbEST |
| Sequence 3744 | AA310181 | dbEST |
| Sequence 3745 | AA310292 | dbEST |
| Sequence 3746 | AA310460 | dbEST |
| Sequence 3747 | AA310561 | dbEST |
| Sequence 3748 | AA310591 | dbEST |
| Sequence 3749 | AA310928 | dbEST |
| Sequence 3750 | AA311044 | dbEST |
| Sequence 3751 | AA311227 | dbEST |
| Sequence 3752 | AA312177 | dbEST |
| Sequence 3753 | AA312242 | dbEST |
| Sequence 3754 | AA312444 | dbEST |
| Sequence 3755 | AA312448 | dbEST |
| Sequence 3756 | AA312503 | dbEST |
| Sequence 3757 | AA313200 | dbEST |
| Sequence 3758 | AA313208 | dbEST |
| Sequence 3759 | AA313418 | dbEST |
| Sequence 3760 | AA313549 | dbEST |
| Sequence 3761 | AA313828 | dbEST |
| Sequence 3762 | AA313837 | dbEST |
| Sequence 3763 | AA313933 | dbEST |
| Sequence 3764 | AA313996 | dbEST |
| Sequence 3765 | AA314061 | dbEST |
| Sequence 3766 | AA314181 | dbEST |
| Sequence 3767 | AA314188 | dbEST |
| Sequence 3768 | AA314202 | dbEST |
| Sequence 3769 | AA314241 | dbEST |
| Sequence 3770 | AA314355 | dbEST |
| Sequence 3771 | AA314465 | dbEST |
| Sequence 3772 | AA314748 | dbEST |
| Sequence 3773 | AA314757 | dbEST |
| Sequence 3774 | AA314847 | dbEST |
| Sequence 3775 | AA314862 | dbEST |
| Sequence 3776 | AA314882 | dbEST |
| Sequence 3777 | AA314961 | dbEST |
| Sequence 3778 | AA315069 | dbEST |
| Sequence 3779 | AA315172 | dbEST |
| Sequence 3780 | AA315215 | dbEST |
| Sequence 3781 | AA315321 | dbEST |
| Sequence 3782 | AA315406 | dbEST |
| Sequence 3783 | AA315444 | dbEST |
| Sequence 3784 | AA315497 | dbEST |
| Sequence 3785 | AA315762 | dbEST |
| Sequence 3786 | AA315950 | dbEST |
| Sequence 3787 | AA315993 | dbEST |
| Sequence 3788 | AA316199 | dbEST |
| Sequence 3789 | AA316322 | dbEST |
| Sequence 3790 | AA317096 | dbEST |
| Sequence 3791 | AA317937 | dbEST |
| Sequence 3792 | AA317956 | dbEST |
| Sequence 3793 | AA318544 | dbEST |
| Sequence 3794 | AA319139 | dbEST |
| Sequence 3795 | AA319538 | dbEST |
| Sequence 3796 | AA320701 | dbEST |
| Sequence 3797 | AA322246 | dbEST |
| Sequence 3798 | AA324251 | dbEST |
| Sequence 3799 | AA324411 | dbEST |
| Sequence 3800 | AA324705 | dbEST |
| Sequence 3801 | AA324831 | dbEST |
| Sequence 3802 | AA325221 | dbEST |
| Sequence 3803 | AA325658 | dbEST |
| Sequence 3804 | AA326793 | dbEST |
| Sequence 3805 | AA327228 | dbEST |
| Sequence 3806 | AA327546 | dbEST |
| Sequence 3807 | AA327611 | dbEST |
| Sequence 3808 | AA330234 | dbEST |
| Sequence 3809 | AA330784 | dbEST |
| Sequence 3810 | AA331859 | dbEST |
| Sequence 3811 | AA332556 | dbEST |
| Sequence 3812 | AA332667 | dbEST |
| Sequence 3813 | AA333307 | dbEST |
| Sequence 3814 | AA333526 | dbEST |
| Sequence 3815 | AA334452 | dbEST |
| Sequence 3816 | AA336081 | dbEST |
| Sequence 3817 | AA336683 | dbEST |
| Sequence 3818 | AA336740 | dbEST |
| Sequence 3819 | AA338257 | dbEST |
| Sequence 3820 | AA339065 | dbEST |
| Sequence 3821 | AA339151 | dbEST |
| Sequence 3822 | AA340654 | dbEST |
| Sequence 3823 | AA340719 | dbEST |
| Sequence 3824 | AA344393 | dbEST |
| Sequence 3825 | AA345558 | dbEST |
| Sequence 3826 | AA346918 | dbEST |
| Sequence 3827 | AA347293 | dbEST |
| Sequence 3828 | AA347390 | dbEST |
| Sequence 3829 | AA347889 | dbEST |
| Sequence 3830 | AA349118 | dbEST |
| Sequence 3831 | AA350206 | dbEST |
| Sequence 3832 | AA350228 | dbEST |
| Sequence 3833 | AA350661 | dbEST |
| Sequence 3834 | AA352755 | dbEST |
| Sequence 3835 | AA354391 | dbEST |
| Sequence 3836 | AA355003 | dbEST |
| Sequence 3837 | AA355196 | dbEST |
| Sequence 3838 | AA355363 | dbEST |
| Sequence 3839 | AA355451 | dbEST |
| Sequence 3840 | AA359003 | dbEST |
| Sequence 3841 | AA359284 | dbEST |
| Sequence 3842 | AA359514 | dbEST |
| Sequence 3843 | AA361042 | dbEST |
| Sequence 3844 | AA362778 | dbEST |
| Sequence 3845 | AA362825 | dbEST |
| Sequence 3846 | AA363142 | dbEST |
| Sequence 3847 | AA364498 | dbEST |
| Sequence 3848 | AA365150 | dbEST |
| Sequence 3849 | AA366518 | dbEST |
| Sequence 3850 | AA367581 | dbEST |
| Sequence 3851 | AA368810 | dbEST |
| Sequence 3852 | AA371017 | dbEST |
| Sequence 3853 | AA371265 | dbEST |
| Sequence 3854 | AA373756 | dbEST |
| Sequence 3855 | AA374222 | dbEST |
| Sequence 3856 | AA374766 | dbEST |
| Sequence 3857 | AA375483 | dbEST |
| Sequence 3858 | AA377218 | dbEST |
| Sequence 3859 | AA378059 | dbEST |
| Sequence 3860 | AA378226 | dbEST |
| Sequence 3861 | AA379015 | dbEST |
| Sequence 3862 | AA385388 | dbEST |
| Sequence 3863 | AA386267 | dbEST |
| Sequence 3864 | AA393259 | dbEST |
| Sequence 3865 | AA393906 | dbEST |
| Sequence 3866 | AA394176 | dbEST |
| Sequence 3867 | AA394299 | dbEST |
| Sequence 3868 | AA397802 | dbEST |
| Sequence 3869 | AA399239 | dbEST |
| Sequence 3870 | AA399393 | dbEST |
| Sequence 3871 | AA399637 | dbEST |
| Sequence 3872 | AA401603 | dbEST |
| Sequence 3873 | AA401957 | dbEST |
| Sequence 3874 | AA402792 | dbEST |
| Sequence 3875 | AA403235 | dbEST |
| Sequence 3876 | AA403322 | dbEST |
| Sequence 3877 | AA404444 | dbEST |
| Sequence 3878 | AA404642 | dbEST |
| Sequence 3879 | AA404646 | dbEST |
| Sequence 3880 | AA405640 | dbEST |
| Sequence 3881 | AA406402 | dbEST |
| Sequence 3882 | AA410700 | dbEST |
| Sequence 3883 | AA411201 | dbEST |
| Sequence 3884 | AA416781 | dbEST |
| Sequence 3885 | AA418473 | dbEST |
| Sequence 3886 | AA420523 | dbEST |
| Sequence 3887 | AA420647 | dbEST |
| Sequence 3888 | AA420690 | dbEST |
| Sequence 3889 | AA420845 | dbEST |
| Sequence 3890 | AA420852 | dbEST |
| Sequence 3891 | AA421213 | dbEST |
| Sequence 3892 | AA421274 | dbEST |
| Sequence 3893 | AA421545 | dbEST |
| Sequence 3894 | AA424561 | dbEST |
| Sequence 3895 | AA424655 | dbEST |
| Sequence 3896 | AA424658 | dbEST |
| Sequence 3897 | AA425613 | dbEST |
| Sequence 3898 | AA426235 | dbEST |
| Sequence 3899 | AA426391 | dbEST |
| Sequence 3900 | AA427471 | dbEST |
| Sequence 3901 | AA427596 | dbEST |
| Sequence 3902 | AA428096 | dbEST |
| Sequence 3903 | AA429173 | dbEST |
| Sequence 3904 | AA430304 | dbEST |
| Sequence 3905 | AA430478 | dbEST |
| Sequence 3906 | AA430597 | dbEST |
| Sequence 3907 | AA430628 | dbEST |
| Sequence 3908 | AA434028 | dbEST |
| Sequence 3909 | AA436008 | dbEST |
| Sequence 3910 | AA436762 | dbEST |
| Sequence 3911 | AA436769 | dbEST |
| Sequence 3912 | AA437277 | dbEST |
| Sequence 3913 | AA442287 | dbEST |
| Sequence 3914 | AA442517 | dbEST |
| Sequence 3915 | AA442687 | dbEST |
| Sequence 3916 | AA443659 | dbEST |
| Sequence 3917 | AA446071 | dbEST |
| Sequence 3918 | AA446716 | dbEST |
| Sequence 3919 | AA446958 | dbEST |
| Sequence 3920 | AA447885 | dbEST |
| Sequence 3921 | AA448118 | dbEST |
| Sequence 3922 | AA448950 | dbEST |
| Sequence 3923 | AA450168 | dbEST |
| Sequence 3924 | AA451733 | dbEST |
| Sequence 3925 | AA452012 | dbEST |
| Sequence 3926 | AA453445 | dbEST |
| Sequence 3927 | AA453562 | dbEST |
| Sequence 3928 | AA454027 | dbEST |
| Sequence 3929 | AA454065 | dbEST |
| Sequence 3930 | AA454912 | dbEST |
| Sequence 3931 | AA455299 | dbEST |
| Sequence 3932 | AA456372 | dbEST |
| Sequence 3933 | AA457021 | dbEST |
| Sequence 3934 | AA458804 | dbEST |
| Sequence 3935 | AA459528 | dbEST |
| Sequence 3936 | AA460511 | dbEST |
| Sequence 3937 | AA460755 | dbEST |
| Sequence 3938 | AA460969 | dbEST |
| Sequence 3939 | AA464763 | dbEST |
| Sequence 3940 | AA465494 | dbEST |
| Sequence 3941 | AA465697 | dbEST |
| Sequence 3942 | AA467864 | dbEST |
| Sequence 3943 | AA467974 | dbEST |
| Sequence 3944 | AA468839 | dbEST |
| Sequence 3945 | AA469129 | dbEST |
| Sequence 3946 | AA469238 | dbEST |
| Sequence 3947 | AA469304 | dbEST |
| Sequence 3948 | AA469406 | dbEST |
| Sequence 3949 | AA471070 | dbEST |
| Sequence 3950 | AA476207 | dbEST |
| Sequence 3951 | AA476543 | dbEST |
| Sequence 3952 | AA476648 | dbEST |
| Sequence 3953 | AA476679 | dbEST |
| Sequence 3954 | AA477579 | dbEST |
| Sequence 3955 | AA477848 | dbEST |
| Sequence 3956 | AA478298 | dbEST |
| Sequence 3957 | AA478522 | dbEST |
| Sequence 3958 | AA478647 | dbEST |
| Sequence 3959 | AA479287 | dbEST |
| Sequence 3960 | AA479809 | dbEST |
| Sequence 3961 | AA480051 | dbEST |
| Sequence 3962 | AA482522 | dbEST |
| Sequence 3963 | AA482884 | dbEST |
| Sequence 3964 | AA484104 | dbEST |
| Sequence 3965 | AA484284 | dbEST |
| Sequence 3966 | AA484568 | dbEST |
| Sequence 3967 | AA484752 | dbEST |
| Sequence 3968 | AA485853 | dbEST |
| Sequence 3969 | AA486838 | dbEST |
| Sequence 3970 | AA488014 | dbEST |
| Sequence 3971 | AA488592 | dbEST |
| Sequence 3972 | AA488954 | dbEST |
| Sequence 3973 | AA489124 | dbEST |
| Sequence 3974 | AA489160 | dbEST |
| Sequence 3975 | AA489323 | dbEST |
| Sequence 3976 | AA491983 | dbEST |
| Sequence 3977 | AA492042 | dbEST |
| Sequence 3978 | AA492256 | dbEST |
| Sequence 3979 | AA494295 | dbEST |
| Sequence 3980 | AA494489 | dbEST |
| Sequence 3981 | AA503040 | dbEST |
| Sequence 3982 | AA503330 | dbEST |
| Sequence 3983 | AA503943 | dbEST |
| Sequence 3984 | AA504703 | dbEST |
| Sequence 3985 | AA504969 | dbEST |
| Sequence 3986 | AA505327 | dbEST |
| Sequence 3987 | AA505568 | dbEST |
| Sequence 3988 | AA506299 | dbEST |
| Sequence 3989 | AA506304 | dbEST |
| Sequence 3990 | AA506459 | dbEST |
| Sequence 3991 | AA507065 | dbEST |
| Sequence 3992 | AA507201 | dbEST |
| Sequence 3993 | AA507217 | dbEST |
| Sequence 3994 | AA507472 | dbEST |
| Sequence 3995 | AA507595 | dbEST |
| Sequence 3996 | AA508861 | dbEST |
| Sequence 3997 | AA513597 | dbEST |
| Sequence 3998 | AA514775 | dbEST |
| Sequence 3999 | AA515132 | dbEST |
| Sequence 4000 | AA516441 | dbEST |
| Sequence 4001 | AA522473 | dbEST |
| Sequence 4002 | AA523006 | dbEST |
| Sequence 4003 | AA523902 | dbEST |
| Sequence 4004 | AA524258 | dbEST |
| Sequence 4005 | AA525497 | dbEST |
| Sequence 4006 | AA525801 | dbEST |
| Sequence 4007 | AA526187 | dbEST |
| Sequence 4008 | AA526472 | dbEST |
| Sequence 4009 | AA526894 | dbEST |
| Sequence 4010 | AA528202 | dbEST |
| Sequence 4011 | AA531561 | dbEST |
| Sequence 4012 | AA532461 | dbEST |
| Sequence 4013 | AA532852 | dbEST |
| Sequence 4014 | AA533001 | dbEST |
| Sequence 4015 | AA533940 | dbEST |
| Sequence 4016 | AA534419 | dbEST |
| Sequence 4017 | AA534543 | dbEST |
| Sequence 4018 | AA535610 | dbEST |
| Sequence 4019 | AA535695 | dbEST |
| Sequence 4020 | AA535837 | dbEST |
| Sequence 4021 | AA536175 | dbEST |
| Sequence 4022 | AA548238 | dbEST |
| Sequence 4023 | AA548600 | dbEST |
| Sequence 4024 | AA551065 | dbEST |
| Sequence 4025 | AA551252 | dbEST |
| Sequence 4026 | AA551773 | dbEST |
| Sequence 4027 | AA552154 | dbEST |
| Sequence 4028 | AA552253 | dbEST |
| Sequence 4029 | AA552570 | dbEST |
| Sequence 4030 | AA555160 | dbEST |
| Sequence 4031 | AA557888 | dbEST |
| Sequence 4032 | AA558976 | dbEST |
| Sequence 4033 | AA564005 | dbEST |
| Sequence 4034 | AA564017 | dbEST |
| Sequence 4035 | AA565044 | dbEST |
| Sequence 4036 | AA565448 | dbEST |
| Sequence 4037 | AA569247 | dbEST |
| Sequence 4038 | AA569354 | dbEST |
| Sequence 4039 | AA569439 | dbEST |
| Sequence 4040 | AA570171 | dbEST |
| Sequence 4041 | AA570182 | dbEST |
| Sequence 4042 | AA572808 | dbEST |
| Sequence 4043 | AA572938 | dbEST |
| Sequence 4044 | AA573557 | dbEST |
| Sequence 4045 | AA573559 | dbEST |
| Sequence 4046 | AA573575 | dbEST |
| Sequence 4047 | AA573737 | dbEST |
| Sequence 4048 | AA573762 | dbEST |
| Sequence 4049 | AA573787 | dbEST |
| Sequence 4050 | AA573811 | dbEST |
| Sequence 4051 | AA573817 | dbEST |
| Sequence 4052 | AA573910 | dbEST |
| Sequence 4053 | AA576252 | dbEST |
| Sequence 4054 | AA576404 | dbEST |
| Sequence 4055 | AA578265 | dbEST |
| Sequence 4056 | AA578773 | dbEST |
| Sequence 4057 | AA578904 | dbEST |
| Sequence 4058 | AA578976 | dbEST |
| Sequence 4059 | AA579596 | dbEST |
| Sequence 4060 | AA581523 | dbEST |
| Sequence 4061 | AA582489 | dbEST |
| Sequence 4062 | AA582728 | dbEST |
| Sequence 4063 | AA582805 | dbEST |
| Sequence 4064 | AA583801 | dbEST |
| Sequence 4065 | AA586744 | dbEST |
| Sequence 4066 | AA588083 | dbEST |
| Sequence 4067 | AA588358 | dbEST |
| Sequence 4068 | AA588801 | dbEST |
| Sequence 4069 | AA592947 | dbEST |
| Sequence 4070 | AA593385 | dbEST |
| Sequence 4071 | AA593879 | dbEST |
| Sequence 4072 | AA595193 | dbEST |
| Sequence 4073 | AA595471 | dbEST |
| Sequence 4074 | AA595582 | dbEST |
| Sequence 4075 | AA598598 | dbEST |
| Sequence 4076 | AA599229 | dbEST |
| Sequence 4077 | AA599650 | dbEST |
| Sequence 4078 | AA602472 | dbEST |
| Sequence 4079 | AA602773 | dbEST |
| Sequence 4080 | AA602979 | dbEST |
| Sequence 4081 | AA603572 | dbEST |
| Sequence 4082 | AA610734 | dbEST |
| Sequence 4083 | AA614274 | dbEST |
| Sequence 4084 | AA620334 | dbEST |
| Sequence 4085 | AA630382 | dbEST |
| Sequence 4086 | AA631152 | dbEST |
| Sequence 4087 | AA632954 | dbEST |
| Sequence 4088 | AA633399 | dbEST |
| Sequence 4089 | AA633909 | dbEST |
| Sequence 4090 | AA640458 | dbEST |
| Sequence 4091 | AA640716 | dbEST |
| Sequence 4092 | AA641074 | dbEST |
| Sequence 4093 | AA644678 | dbEST |
| Sequence 4094 | AA648209 | dbEST |
| Sequence 4095 | AA649298 | dbEST |
| Sequence 4096 | AA650241 | dbEST |
| Sequence 4097 | AA654709 | dbEST |
| Sequence 4098 | AA658207 | dbEST |
| Sequence 4099 | AA659179 | dbEST |
| Sequence 4100 | AA659725 | dbEST |
| Sequence 4101 | AA661464 | dbEST |
| Sequence 4102 | AA661927 | dbEST |
| Sequence 4103 | AA664482 | dbEST |
| Sequence 4104 | AA669484 | dbEST |
| Sequence 4105 | AA669544 | dbEST |
| Sequence 4106 | AA675892 | dbEST |
| Sequence 4107 | AA678873 | dbEST |
| Sequence 4108 | AA683546 | dbEST |
| Sequence 4109 | AA687733 | dbEST |
| Sequence 4110 | AA693922 | dbEST |
| Sequence 4111 | AA694186 | dbEST |
| Sequence 4112 | AA694477 | dbEST |
| Sequence 4113 | AA700876 | dbEST |
| Sequence 4114 | AA703004 | dbEST |
| Sequence 4115 | AA703925 | dbEST |
| Sequence 4116 | AA704690 | dbEST |
| Sequence 4117 | AA705054 | dbEST |
| Sequence 4118 | AA706797 | dbEST |
| Sequence 4119 | AA709070 | dbEST |
| Sequence 4120 | AA713760 | dbEST |
| Sequence 4121 | AA714902 | dbEST |
| Sequence 4122 | AA715036 | dbEST |
| Sequence 4123 | AA719530 | dbEST |
| Sequence 4124 | AA721298 | dbEST |
| Sequence 4125 | AA722090 | dbEST |
| Sequence 4126 | AA744876 | dbEST |
| Sequence 4127 | AA757282 | dbEST |
| Sequence 4128 | AA758023 | dbEST |
| Sequence 4129 | AA761562 | dbEST |
| Sequence 4130 | AA761831 | dbEST |
| Sequence 4131 | AA769102 | dbEST |
| Sequence 4132 | AA769142 | dbEST |
| Sequence 4133 | AA770690 | dbEST |
| Sequence 4134 | AA772736 | dbEST |
| Sequence 4135 | AA775824 | dbEST |
| Sequence 4136 | AA776709 | dbEST |
| Sequence 4137 | AA776740 | dbEST |
| Sequence 4138 | AA777265 | dbEST |
| Sequence 4139 | AA777489 | dbEST |
| Sequence 4140 | AA778571 | dbEST |
| Sequence 4141 | AA804879 | dbEST |
| Sequence 4142 | AA808146 | dbEST |
| Sequence 4143 | AA808466 | dbEST |
| Sequence 4144 | AA813108 | dbEST |
| Sequence 4145 | AA813287 | dbEST |
| Sequence 4146 | AA814412 | dbEST |
| Sequence 4147 | AA814634 | dbEST |
| Sequence 4148 | AA815173 | dbEST |
| Sequence 4149 | AA824395 | dbEST |
| Sequence 4150 | AA826491 | dbEST |
| Sequence 4151 | AA832480 | dbEST |
| Sequence 4152 | AA835490 | dbEST |
| Sequence 4153 | AA836233 | dbEST |
| Sequence 4154 | AA838431 | dbEST |
| Sequence 4155 | AA843216 | dbEST |
| Sequence 4156 | AA843683 | dbEST |
| Sequence 4157 | AA844081 | dbEST |
| Sequence 4158 | AA844230 | dbEST |
| Sequence 4159 | AA854305 | dbEST |
| Sequence 4160 | AA856914 | dbEST |
| Sequence 4161 | AA864975 | dbEST |
| Sequence 4162 | AA868337 | dbEST |
| Sequence 4163 | AA873459 | dbEST |
| Sequence 4164 | AA877796 | dbEST |
| Sequence 4165 | AA877864 | dbEST |
| Sequence 4166 | AA883132 | dbEST |
| Sequence 4167 | AA883580 | dbEST |
| Sequence 4168 | AA886497 | dbEST |
| Sequence 4169 | AA896959 | dbEST |
| Sequence 4170 | AA902256 | dbEST |
| Sequence 4171 | AA907068 | dbEST |
| Sequence 4172 | AA926992 | dbEST |
| Sequence 4173 | AA932059 | dbEST |
| Sequence 4174 | AA933573 | dbEST |
| Sequence 4175 | AA936089 | dbEST |
| Sequence 4176 | AA938156 | dbEST |
| Sequence 4177 | AA947441 | dbEST |
| Sequence 4178 | AA948037 | dbEST |
| Sequence 4179 | AA948223 | dbEST |
| Sequence 4180 | AA954079 | dbEST |
| Sequence 4181 | AA962587 | dbEST |
| Sequence 4182 | AA969613 | dbEST |
| Sequence 4183 | AA970266 | dbEST |
| Sequence 4184 | AA970314 | dbEST |
| Sequence 4185 | AA970570 | dbEST |
| Sequence 4186 | AA977406 | dbEST |
| Sequence 4187 | AA978126 | dbEST |
| Sequence 4188 | AA983832 | dbEST |
| Sequence 4189 | AA984173 | dbEST |
| Sequence 4190 | AA984744 | dbEST |
| Sequence 4191 | AA987442 | dbEST |
| Sequence 4192 | AA988520 | dbEST |
| Sequence 4193 | AB005621 | dbEST |
| Sequence 4194 | AF017688 | dbEST |
| Sequence 4195 | AF062725 | dbEST |
| Sequence 4196 | AF062729 | dbEST |
| Sequence 4197 | AI004596 | dbEST |
| Sequence 4198 | AI015574 | dbEST |
| Sequence 4199 | AI016169 | dbEST |
| Sequence 4200 | AI017022 | dbEST |
| Sequence 4201 | AI017959 | dbEST |
| Sequence 4202 | AI018617 | dbEST |
| Sequence 4203 | AI024907 | dbEST |
| Sequence 4204 | AI025376 | dbEST |
| Sequence 4205 | AI027516 | dbEST |
| Sequence 4206 | AI034024 | dbEST |
| Sequence 4207 | AI038048 | dbEST |
| Sequence 4208 | AI039227 | dbEST |
| Sequence 4209 | AI039301 | dbEST |
| Sequence 4210 | AI039813 | dbEST |
| Sequence 4211 | AI040339 | dbEST |
| Sequence 4212 | AI042002 | dbEST |
| Sequence 4213 | AI049971 | dbEST |
| Sequence 4214 | AI050826 | dbEST |
| Sequence 4215 | AI051957 | dbEST |
| Sequence 4216 | AI052061 | dbEST |
| Sequence 4217 | AI061610 | dbEST |
| Sequence 4218 | AI064931 | dbEST |
| Sequence 4219 | AI075041 | dbEST |
| Sequence 4220 | AI077488 | dbEST |
| Sequence 4221 | AI077719 | dbEST |
| Sequence 4222 | AI078217 | dbEST |
| Sequence 4223 | AI081133 | dbEST |
| Sequence 4224 | AI081379 | dbEST |
| Sequence 4225 | AI081945 | dbEST |
| Sequence 4226 | AI082235 | dbEST |
| Sequence 4227 | AI085263 | dbEST |
| Sequence 4228 | AI085381 | dbEST |
| Sequence 4229 | AI087080 | dbEST |
| Sequence 4230 | AI088914 | dbEST |
| Sequence 4231 | AI089875 | dbEST |
| Sequence 4232 | AI090012 | dbEST |
| Sequence 4233 | AI090827 | dbEST |
| Sequence 4234 | AI091136 | dbEST |
| Sequence 4235 | AI091516 | dbEST |
| Sequence 4236 | AI091814 | dbEST |
| Sequence 4237 | AI094634 | dbEST |
| Sequence 4238 | AI097214 | dbEST |
| Sequence 4239 | AI114433 | dbEST |
| Sequence 4240 | AI114651 | dbEST |
| Sequence 4241 | AI125166 | dbEST |
| Sequence 4242 | AI126653 | dbEST |
| Sequence 4243 | AI127526 | dbEST |
| Sequence 4244 | AI127556 | dbEST |
| Sequence 4245 | AI128883 | dbEST |
| Sequence 4246 | AI128949 | dbEST |
| Sequence 4247 | AI131444 | dbEST |
| Sequence 4248 | AI133208 | dbEST |
| Sequence 4249 | AI139036 | dbEST |
| Sequence 4250 | AI139394 | dbEST |
| Sequence 4251 | AI140096 | dbEST |
| Sequence 4252 | AI140210 | dbEST |
| Sequence 4253 | AI141189 | dbEST |
| Sequence 4254 | AI141583 | dbEST |
| Sequence 4255 | AI141732 | dbEST |
| Sequence 4256 | AI148251 | dbEST |
| Sequence 4257 | AI150619 | dbEST |
| Sequence 4258 | AI160426 | dbEST |
| Sequence 4259 | AI161253 | dbEST |
| Sequence 4260 | AI167801 | dbEST |
| Sequence 4261 | AI184320 | dbEST |
| Sequence 4262 | AI189339 | dbEST |
| Sequence 4263 | AI189345 | dbEST |
| Sequence 4264 | AI189386 | dbEST |
| Sequence 4265 | AI189894 | dbEST |
| Sequence 4266 | AI190648 | dbEST |
| Sequence 4267 | AI192780 | dbEST |
| Sequence 4268 | AI192784 | dbEST |
| Sequence 4269 | AI193675 | dbEST |
| Sequence 4270 | AI198311 | dbEST |
| Sequence 4271 | AI198771 | dbEST |
| Sequence 4272 | AI198986 | dbEST |
| Sequence 4273 | AI199641 | dbEST |
| Sequence 4274 | AI199681 | dbEST |
| Sequence 4275 | AI199773 | dbEST |
| Sequence 4276 | AI200324 | dbEST |
| Sequence 4277 | AI200444 | dbEST |
| Sequence 4278 | AI201261 | dbEST |
| Sequence 4279 | AI203141 | dbEST |
| Sequence 4280 | AI203350 | dbEST |
| Sequence 4281 | AI207650 | dbEST |
| Sequence 4282 | AI214043 | dbEST |
| Sequence 4283 | AI216966 | dbEST |
| Sequence 4284 | AI216986 | dbEST |
| Sequence 4285 | AI216988 | dbEST |
| Sequence 4286 | AI217003 | dbEST |
| Sequence 4287 | AI217009 | dbEST |
| Sequence 4288 | AI217021 | dbEST |
| Sequence 4289 | AI219174 | dbEST |
| Sequence 4290 | AI244335 | dbEST |
| Sequence 4291 | AI246167 | dbEST |
| Sequence 4292 | AI248514 | dbEST |
| Sequence 4293 | AI253288 | dbEST |
| Sequence 4294 | AI253300 | dbEST |
| Sequence 4295 | AI253304 | dbEST |
| Sequence 4296 | AI253319 | dbEST |
| Sequence 4297 | AI253330 | dbEST |
| Sequence 4298 | AI253335 | dbEST |
| Sequence 4299 | AI253367 | dbEST |
| Sequence 4300 | AI253436 | dbEST |
| Sequence 4301 | AI261763 | dbEST |
| Sequence 4302 | AI262139 | dbEST |
| Sequence 4303 | AI267158 | dbEST |
| Sequence 4304 | AI267162 | dbEST |
| Sequence 4305 | AI267185 | dbEST |
| Sequence 4306 | AI267282 | dbEST |
| Sequence 4307 | AI267307 | dbEST |
| Sequence 4308 | AI267397 | dbEST |
| Sequence 4309 | AI267502 | dbEST |
| Sequence 4310 | AI267574 | dbEST |
| Sequence 4311 | AI267596 | dbEST |
| Sequence 4312 | AI267652 | dbEST |
| Sequence 4313 | AI267677 | dbEST |
| Sequence 4314 | AI267808 | dbEST |
| Sequence 4315 | AI267941 | dbEST |
| Sequence 4316 | AI269883 | dbEST |
| Sequence 4317 | AI271740 | dbEST |
| Sequence 4318 | AI274786 | dbEST |
| Sequence 4319 | AI275114 | dbEST |
| Sequence 4320 | AI275175 | dbEST |
| Sequence 4321 | AI277434 | dbEST |
| Sequence 4322 | AI277739 | dbEST |
| Sequence 4323 | AI279141 | dbEST |
| Sequence 4324 | AI281882 | dbEST |
| Sequence 4325 | AI284011 | dbEST |
| Sequence 4326 | AI287972 | dbEST |
| Sequence 4327 | AI288388 | dbEST |
| Sequence 4328 | AI288730 | dbEST |
| Sequence 4329 | AI288929 | dbEST |
| Sequence 4330 | AI289120 | dbEST |
| Sequence 4331 | AI291583 | dbEST |
| Sequence 4332 | AI292299 | dbEST |
| Sequence 4333 | AI306464 | dbEST |
| Sequence 4334 | AI309401 | dbEST |
| Sequence 4335 | AI312049 | dbEST |
| Sequence 4336 | AI332904 | dbEST |
| Sequence 4337 | AI333548 | dbEST |
| Sequence 4338 | AI335269 | dbEST |
| Sequence 4339 | AI340246 | dbEST |
| Sequence 4340 | AI341076 | dbEST |
| Sequence 4341 | AI341334 | dbEST |
| Sequence 4342 | AI341352 | dbEST |
| Sequence 4343 | AI343091 | dbEST |
| Sequence 4344 | AI343604 | dbEST |
| Sequence 4345 | AI346657 | dbEST |
| Sequence 4346 | AI346809 | dbEST |
| Sequence 4347 | AI347155 | dbEST |
| Sequence 4348 | AI349452 | dbEST |
| Sequence 4349 | AI350388 | dbEST |
| Sequence 4350 | AI352032 | dbEST |
| Sequence 4351 | AI356718 | dbEST |
| Sequence 4352 | AI360870 | dbEST |
| Sequence 4353 | AI361965 | dbEST |
| Sequence 4354 | AI362490 | dbEST |
| Sequence 4355 | AI365603 | dbEST |
| Sequence 4356 | AI366365 | dbEST |
| Sequence 4357 | AI366376 | dbEST |
| Sequence 4358 | AI372599 | dbEST |
| Sequence 4359 | AI373323 | dbEST |
| Sequence 4360 | AI375465 | dbEST |
| Sequence 4361 | AI381996 | dbEST |
| Sequence 4362 | AI394173 | dbEST |
| Sequence 4363 | AI394498 | dbEST |
| Sequence 4364 | AI417833 | dbEST |
| Sequence 4365 | AI421412 | dbEST |
| Sequence 4366 | AI432603 | dbEST |
| Sequence 4367 | AI434025 | dbEST |
| Sequence 4368 | AI434639 | dbEST |
| Sequence 4369 | AI436714 | dbEST |
| Sequence 4370 | AI439173 | dbEST |
| Sequence 4371 | AI453358 | dbEST |
| Sequence 4372 | AI473779 | dbEST |
| Sequence 4373 | AI476254 | dbEST |
| Sequence 4374 | AI499378 | dbEST |
| Sequence 4375 | AI499439 | dbEST |
| Sequence 4376 | AI499933 | dbEST |
| Sequence 4377 | AI521270 | dbEST |
| Sequence 4378 | AI525426 | dbEST |
| Sequence 4379 | AI525773 | dbEST |
| Sequence 4380 | AI525843 | dbEST |
| Sequence 4381 | AI537498 | dbEST |
| Sequence 4382 | AI538681 | dbEST |
| Sequence 4383 | AI547284 | dbEST |
| Sequence 4384 | AI547309 | dbEST |
| Sequence 4385 | AI557226 | dbEST |
| Sequence 4386 | AI557495 | dbEST |
| Sequence 4387 | AI557626 | dbEST |
| Sequence 4388 | AI559999 | dbEST |
| Sequence 4389 | AI566084 | dbEST |
| Sequence 4390 | AI566490 | dbEST |
| Sequence 4391 | AI570942 | dbEST |
| Sequence 4392 | AI571069 | dbEST |
| Sequence 4393 | AI580160 | dbEST |
| Sequence 4394 | AI587118 | dbEST |
| Sequence 4395 | AI588087 | dbEST |
| Sequence 4396 | AI589633 | dbEST |
| Sequence 4397 | AI608968 | dbEST |
| Sequence 4398 | AI628609 | dbEST |
| Sequence 4399 | AI630282 | dbEST |
| Sequence 4400 | AI636577 | dbEST |
| Sequence 4401 | AI636911 | dbEST |
| Sequence 4402 | AI638327 | dbEST |
| Sequence 4403 | AI648682 | dbEST |
| Sequence 4404 | AI651069 | dbEST |
| Sequence 4405 | AI651974 | dbEST |
| Sequence 4406 | AI651976 | dbEST |
| Sequence 4407 | AI652084 | dbEST |
| Sequence 4408 | AI652555 | dbEST |
| Sequence 4409 | AI660243 | dbEST |
| Sequence 4410 | AI669253 | dbEST |
| Sequence 4411 | AI670002 | dbEST |
| Sequence 4412 | AI672362 | dbEST |
| Sequence 4413 | AI674583 | dbEST |
| Sequence 4414 | AI675889 | dbEST |
| Sequence 4415 | AI679629 | dbEST |
| Sequence 4416 | AI683183 | dbEST |
| Sequence 4417 | AI684039 | dbEST |
| Sequence 4418 | AI684170 | dbEST |
| Sequence 4419 | AI686957 | dbEST |
| Sequence 4420 | AI690236 | dbEST |
| Sequence 4421 | AI692940 | dbEST |
| Sequence 4422 | AI692995 | dbEST |
| Sequence 4423 | AI694572 | dbEST |
| Sequence 4424 | AI697815 | dbEST |
| Sequence 4425 | AI701119 | dbEST |
| Sequence 4426 | AI708995 | dbEST |
| Sequence 4427 | AI732541 | dbEST |
| Sequence 4428 | AI732557 | dbEST |
| Sequence 4429 | AI744451 | dbEST |
| Sequence 4430 | AI745280 | dbEST |
| Sequence 4431 | AI751583 | dbEST |
| Sequence 4432 | AI751914 | dbEST |
| Sequence 4433 | AI752822 | dbEST |
| Sequence 4434 | AI753269 | dbEST |
| Sequence 4435 | AI754013 | dbEST |
| Sequence 4436 | AI755085 | dbEST |
| Sequence 4437 | AI761508 | dbEST |
| Sequence 4438 | AI761728 | dbEST |
| Sequence 4439 | AI768848 | dbEST |
| Sequence 4440 | AI769145 | dbEST |
| Sequence 4441 | AI791719 | dbEST |
| Sequence 4442 | AI802998 | dbEST |
| Sequence 4443 | AI806161 | dbEST |
| Sequence 4444 | AI806996 | dbEST |
| Sequence 4445 | AI815416 | dbEST |
| Sequence 4446 | AI815449 | dbEST |
| Sequence 4447 | AI815497 | dbEST |
| Sequence 4448 | AI815829 | dbEST |
| Sequence 4449 | AI815966 | dbEST |
| Sequence 4450 | AI815976 | dbEST |
| Sequence 4451 | AI816033 | dbEST |
| Sequence 4452 | AI820995 | dbEST |
| Sequence 4453 | AI829303 | dbEST |
| Sequence 4454 | AI858066 | dbEST |
| Sequence 4455 | AI860714 | dbEST |
| Sequence 4456 | AI866262 | dbEST |
| Sequence 4457 | AI871477 | dbEST |
| Sequence 4458 | AI872085 | dbEST |
| Sequence 4459 | AI878878 | dbEST |
| Sequence 4460 | AI879010 | dbEST |
| Sequence 4461 | AI879059 | dbEST |
| Sequence 4462 | AI879242 | dbEST |
| Sequence 4463 | AI884578 | dbEST |
| Sequence 4464 | AI885833 | dbEST |
| Sequence 4465 | AI886418 | dbEST |
| Sequence 4466 | AI887517 | dbEST |
| Sequence 4467 | AI887664 | dbEST |
| Sequence 4468 | AI911703 | dbEST |
| Sequence 4469 | AI914133 | dbEST |
| Sequence 4470 | AI917649 | dbEST |
| Sequence 4471 | AI924598 | dbEST |
| Sequence 4472 | AI929113 | dbEST |
| Sequence 4473 | AI929453 | dbEST |
| Sequence 4474 | AI934153 | dbEST |
| Sequence 4475 | AI934595 | dbEST |
| Sequence 4476 | AI935291 | dbEST |
| Sequence 4477 | AI935816 | dbEST |
| Sequence 4478 | AI937060 | dbEST |
| Sequence 4479 | AI951021 | dbEST |
| Sequence 4480 | AI951536 | dbEST |
| Sequence 4481 | AI954800 | dbEST |
| Sequence 4482 | AI962736 | dbEST |
| Sequence 4483 | AI973126 | dbEST |
| Sequence 4484 | AI973218 | dbEST |
| Sequence 4485 | AI983430 | dbEST |
| Sequence 4486 | AI991281 | dbEST |
| Sequence 4487 | AL035886 | dbEST |
| Sequence 4488 | AL035985 | dbEST |
| Sequence 4489 | AL036026 | dbEST |
| Sequence 4490 | AL036415 | dbEST |
| Sequence 4491 | AL036445 | dbEST |
| Sequence 4492 | AL036763 | dbEST |
| Sequence 4493 | AL036764 | dbEST |
| Sequence 4494 | AL037724 | dbEST |
| Sequence 4495 | AL038058 | dbEST |
| Sequence 4496 | AL038660 | dbEST |
| Sequence 4497 | AL038721 | dbEST |
| Sequence 4498 | AL039517 | dbEST |
| Sequence 4499 | AL039691 | dbEST |
| Sequence 4500 | AL040505 | dbEST |
| Sequence 4501 | AL040993 | dbEST |
| Sequence 4502 | AL041470 | dbEST |
| Sequence 4503 | AL042896 | dbEST |
| Sequence 4504 | AL043245 | dbEST |
| Sequence 4505 | AL044016 | dbEST |
| Sequence 4506 | AL044350 | dbEST |
| Sequence 4507 | AL045492 | dbEST |
| Sequence 4508 | AL046107 | dbEST |
| Sequence 4509 | AL046313 | dbEST |
| Sequence 4510 | AL047277 | dbEST |
| Sequence 4511 | AL047334 | dbEST |
| Sequence 4512 | AL047838 | dbEST |
| Sequence 4513 | AL048554 | dbEST |
| Sequence 4514 | AL110463 | dbEST |
| Sequence 4515 | AL118956 | dbEST |
| Sequence 4516 | AL120807 | dbEST |
| Sequence 4517 | AW006392 | dbEST |
| Sequence 4518 | AW014590 | dbEST |
| Sequence 4519 | AW016611 | dbEST |
| Sequence 4520 | AW026543 | dbEST |
| Sequence 4521 | AW026680 | dbEST |
| Sequence 4522 | AW027007 | dbEST |
| Sequence 4523 | AW027219 | dbEST |
| Sequence 4524 | AW029146 | dbEST |
| Sequence 4525 | AW058559 | dbEST |
| Sequence 4526 | AW062376 | dbEST |
| Sequence 4527 | AW071906 | dbEST |
| Sequence 4528 | AW075433 | dbEST |
| Sequence 4529 | AW084950 | dbEST |
| Sequence 4530 | AW129510 | dbEST |
| Sequence 4531 | AW132009 | dbEST |
| Sequence 4532 | AW134746 | dbEST |
| Sequence 4533 | AW134867 | dbEST |
| Sequence 4534 | AW151254 | dbEST |
| Sequence 4535 | AW152107 | dbEST |
| Sequence 4536 | AW160669 | dbEST |
| Sequence 4537 | AW161467 | dbEST |
| Sequence 4538 | AW161705 | dbEST |
| Sequence 4539 | AW163208 | dbEST |
| Sequence 4540 | AW163441 | dbEST |
| Sequence 4541 | AW167481 | dbEST |
| Sequence 4542 | AW169941 | dbEST |
| Sequence 4543 | AW170485 | dbEST |
| Sequence 4544 | AW176062 | dbEST |
| Sequence 4545 | AW177936 | dbEST |
| Sequence 4546 | AW177948 | dbEST |
| Sequence 4547 | AW177957 | dbEST |
| Sequence 4548 | AW179006 | dbEST |
| Sequence 4549 | AW181932 | dbEST |
| Sequence 4550 | AW183601 | dbEST |
| Sequence 4551 | AW188666 | dbEST |
| Sequence 4552 | C02912 | dbEST |
| Sequence 4553 | C16620 | dbEST |
| Sequence 4554 | C18148 | dbEST |
| Sequence 4555 | C18436 | dbEST |
| Sequence 4556 | C18570 | dbEST |
| Sequence 4557 | D58694 | dbEST |
| Sequence 4558 | F01327 | dbEST |
| Sequence 4559 | F05894 | dbEST |
| Sequence 4560 | F07432 | dbEST |
| Sequence 4561 | F19390 | dbEST |
| Sequence 4562 | F25339 | dbEST |
| Sequence 4563 | F27302 | dbEST |
| Sequence 4564 | F27796 | dbEST |
| Sequence 4565 | F28190 | dbEST |
| Sequence 4566 | F34889 | dbEST |
| Sequence 4567 | H00112 | dbEST |
| Sequence 4568 | H01419 | dbEST |
| Sequence 4569 | H01979 | dbEST |
| Sequence 4570 | H02725 | dbEST |
| Sequence 4571 | H06040 | dbEST |
| Sequence 4572 | H06313 | dbEST |
| Sequence 4573 | H08929 | dbEST |
| Sequence 4574 | H10932 | dbEST |
| Sequence 4575 | H12090 | dbEST |
| Sequence 4576 | H12254 | dbEST |
| Sequence 4577 | H17195 | dbEST |
| Sequence 4578 | H19153 | dbEST |
| Sequence 4579 | H19367 | dbEST |
| Sequence 4580 | H20888 | dbEST |
| Sequence 4581 | H23370 | dbEST |
| Sequence 4582 | H25211 | dbEST |
| Sequence 4583 | H25825 | dbEST |
| Sequence 4584 | H26319 | dbEST |
| Sequence 4585 | H28581 | dbEST |
| Sequence 4586 | H29149 | dbEST |
| Sequence 4587 | H29635 | dbEST |
| Sequence 4588 | H39809 | dbEST |
| Sequence 4589 | H49903 | dbEST |
| Sequence 4590 | H54240 | dbEST |
| Sequence 4591 | H60247 | dbEST |
| Sequence 4592 | H61232 | dbEST |
| Sequence 4593 | H70543 | dbEST |
| Sequence 4594 | H71076 | dbEST |
| Sequence 4595 | H79973 | dbEST |
| Sequence 4596 | H83317 | dbEST |
| Sequence 4597 | H83328 | dbEST |
| Sequence 4598 | H85200 | dbEST |
| Sequence 4599 | H87807 | dbEST |
| Sequence 4600 | H89883 | dbEST |
| Sequence 4601 | H97090 | dbEST |
| Sequence 4602 | H99586 | dbEST |
| Sequence 4603 | N21201 | dbEST |
| Sequence 4604 | N24459 | dbEST |
| Sequence 4605 | N26421 | dbEST |
| Sequence 4606 | N28008 | dbEST |
| Sequence 4607 | N31801 | dbEST |
| Sequence 4608 | N32737 | dbEST |
| Sequence 4609 | N41418 | dbEST |
| Sequence 4610 | N42929 | dbEST |
| Sequence 4611 | N47360 | dbEST |
| Sequence 4612 | N71080 | dbEST |
| Sequence 4613 | R11885 | dbEST |
| Sequence 4614 | R13878 | dbEST |
| Sequence 4615 | R17110 | dbEST |
| Sequence 4616 | R18274 | dbEST |
| Sequence 4617 | R24400 | dbEST |
| Sequence 4618 | R24834 | dbEST |
| Sequence 4619 | R34553 | dbEST |
| Sequence 4620 | R36477 | dbEST |
| Sequence 4621 | R42994 | dbEST |
| Sequence 4622 | R54273 | dbEST |
| Sequence 4623 | R59031 | dbEST |
| Sequence 4624 | R61601 | dbEST |
| Sequence 4625 | R70116 | dbEST |
| Sequence 4626 | R77854 | dbEST |
| Sequence 4627 | R85291 | dbEST |
| Sequence 4628 | T30604 | dbEST |
| Sequence 4629 | T59793 | dbEST |
| Sequence 4630 | T62753 | dbEST |
| Sequence 4631 | T65174 | dbEST |
| Sequence 4632 | T65642 | dbEST |
| Sequence 4633 | T68057 | dbEST |
| Sequence 4634 | T71122 | dbEST |
| Sequence 4635 | T74004 | dbEST |
| Sequence 4636 | T79957 | dbEST |
| Sequence 4637 | T81666 | dbEST |
| Sequence 4638 | T87572 | dbEST |
| Sequence 4639 | W01739 | dbEST |
| Sequence 4640 | W02575 | dbEST |
| Sequence 4641 | W04588 | dbEST |
| Sequence 4642 | W28466 | dbEST |
| Sequence 4643 | W31167 | dbEST |
| Sequence 4644 | W32156 | dbEST |
| Sequence 4645 | W38407 | dbEST |
| Sequence 4646 | W38913 | dbEST |
| Sequence 4647 | W39053 | dbEST |
| Sequence 4648 | W48827 | dbEST |
| Sequence 4649 | W72729 | dbEST |
| Sequence 4650 | W77826 | dbEST |
| Sequence 4651 | W79340 | dbEST |
| Sequence 4652 | W80673 | dbEST |
| Sequence 4653 | W85764 | dbEST |
| Sequence 4654 | Z33579 | dbEST |
| Sequence 4655 | N91259 | NUCPATENT |
| Sequence 4656 | N91825 | NUCPATENT |
| Sequence 4657 | Q37811 | NUCPATENT |
| Sequence 4658 | Q65520 | NUCPATENT |
| Sequence 4659 | Q94780 | NUCPATENT |
| Sequence 4660 | T02792 | NUCPATENT |
| Sequence 4661 | T60740 | NUCPATENT |
| Sequence 4662 | T72175 | NUCPATENT |
| Sequence 4663 | T79274 | NUCPATENT |
| Sequence 4664 | T97897 | NUCPATENT |
| Sequence 4665 | V20443 | NUCPATENT |
| Sequence 4666 | V29267 | NUCPATENT |
| Sequence 4667 | V35457 | NUCPATENT |
| Sequence 4668 | V43612 | NUCPATENT |
| Sequence 4669 | V57903 | NUCPATENT |
| Sequence 4670 | V58544 | NUCPATENT |
| Sequence 4671 | V58619 | NUCPATENT |
| Sequence 4672 | V59096 | NUCPATENT |
| Sequence 4673 | V59539 | NUCPATENT |
| Sequence 4674 | V62427 | NUCPATENT |
| Sequence 4675 | V64158 | NUCPATENT |
| Sequence 4676 | V69855 | NUCPATENT |
| Sequence 4677 | V70354 | NUCPATENT |
| Sequence 4678 | V72211 | NUCPATENT |
| Sequence 4679 | V81414 | NUCPATENT |
| Sequence 4680 | V84510 | NUCPATENT |
| Sequence 4681 | V86232 | NUCPATENT |
| Sequence 4682 | V89565 | NUCPATENT |
| Sequence 4683 | V90009 | NUCPATENT |
| Sequence 4684 | X00630 | NUCPATENT |
| Sequence 4685 | X03841 | NUCPATENT |
| Sequence 4686 | X07429 | NUCPATENT |
| Sequence 4687 | X20426 | NUCPATENT |
| Sequence 4688 | X20437 | NUCPATENT |
| Sequence 4689 | X27262 | NUCPATENT |
| Sequence 4690 | X37385 | NUCPATENT |
| Sequence 4691 | X37553 | NUCPATENT |
| Sequence 4692 | X39825 | NUCPATENT |
| Sequence 4693 | X39912 | NUCPATENT |
| Sequence 4694 | X52268 | NUCPATENT |
| Sequence 4695 | X56415 | NUCPATENT |
| Sequence 4696 | X59352 | NUCPATENT |
| Sequence 4697 | X80676 | NUCPATENT |
| Sequence 4698 | X87397 | NUCPATENT |
| Sequence 4699 | X97772 | NUCPATENT |
| Sequence 4700 | X97790 | NUCPATENT |
| Sequence 4701 | Z06250 | NUCPATENT |
| Sequence 4702 | Z15124 | NUCPATENT |
| Sequence 4703 | Z15132 | NUCPATENT |
| Sequence 4704 | Z15187 | NUCPATENT |
| Sequence 4705 | Z15987 | NUCPATENT |
| Sequence 4706 | Z24892 | NUCPATENT |
| Sequence 4707 | Z24893 | NUCPATENT |
| Sequence 4708 | Z27237 | NUCPATENT |
| Sequence 4709 | Z33949 | NUCPATENT |
| Sequence 4710 | AC29436 | PREPATNUC |
| Sequence 4711 | AC31443 | PREPATNUC |
| Sequence 4712 | AC31770 | PREPATNUC |
| Sequence 4713 | AC33242 | PREPATNUC |
| Sequence 4714 | AC33576 | PREPATNUC |
| Sequence 4715 | AC33673 | PREPATNUC |
| Sequence 4716 | AC33992 | PREPATNUC |
| Sequence 4717 | AC34287 | PREPATNUC |
| Sequence 4718 | AC34371 | PREPATNUC |
| Sequence 4719 | AC34444 | PREPATNUC |
| Sequence 4720 | AC34517 | PREPATNUC |
| Sequence 4721 | AC34738 | PREPATNUC |
| Sequence 4722 | AC34865 | PREPATNUC |
| Sequence 4723 | AC35363 | PREPATNUC |
| Sequence 4724 | A06846 | GENBANK |
| Sequence 4725 | A17546 | GENBANK |
| Sequence 4726 | AB002346 | GENBANK |
| Sequence 4727 | AB002353 | GENBANK |
| Sequence 4728 | AB002375 | GENBANK |
| Sequence 4729 | AB002380 | GENBANK |
| Sequence 4730 | AB002386 | GENBANK |
| Sequence 4731 | AB007929 | GENBANK |
| Sequence 4732 | AB007932 | GENBANK |
| Sequence 4733 | AB007956 | GENBANK |
| Sequence 4734 | AB011120 | GENBANK |
| Sequence 4735 | AB011147 | GENBANK |
| Sequence 4736 | AB014888 | GENBANK |
| Sequence 4737 | AB016533 | GENBANK |
| Sequence 4738 | AB017430 | GENBANK |
| Sequence 4739 | AB017971 | GENBANK |
| Sequence 4740 | AB018284 | GENBANK |
| Sequence 4741 | AB018313 | GENBANK |
| Sequence 4742 | AB019392 | GENBANK |
| Sequence 4743 | AB019568 | GENBANK |
| Sequence 4744 | AB020665 | GENBANK |
| Sequence 4745 | AB020679 | GENBANK |
| Sequence 4746 | AB023145 | GENBANK |
| Sequence 4747 | AB023177 | GENBANK |
| Sequence 4748 | AB023201 | GENBANK |
| Sequence 4749 | AB027466 | GENBANK |
| Sequence 4750 | AB028624 | GENBANK |
| Sequence 4751 | AB028945 | GENBANK |
| Sequence 4752 | AB029290 | GENBANK |
| Sequence 4753 | AB032951 | GENBANK |
| Sequence 4754 | AB033033 | GENBANK |
| Sequence 4755 | AB033076 | GENBANK |
| Sequence 4756 | AB033080 | GENBANK |
| Sequence 4757 | AD001528 | GENBANK |
| Sequence 4758 | AF001893 | GENBANK |
| Sequence 4759 | AF002210 | GENBANK |
| Sequence 4760 | AF004813 | GENBANK |
| Sequence 4761 | AF006043 | GENBANK |
| Sequence 4762 | AF007217 | GENBANK |
| Sequence 4763 | AF009615 | GENBANK |
| Sequence 4764 | AF010233 | GENBANK |
| Sequence 4765 | AF010315 | GENBANK |
| Sequence 4766 | AF012872 | GENBANK |
| Sequence 4767 | AF013160 | GENBANK |
| Sequence 4768 | AF015283 | GENBANK |
| Sequence 4769 | AF017256 | GENBANK |
| Sequence 4770 | AF020202 | GENBANK |
| Sequence 4771 | AF021351 | GENBANK |
| Sequence 4772 | AF028832 | GENBANK |
| Sequence 4773 | AF035295 | GENBANK |
| Sequence 4774 | AF035812 | GENBANK |
| Sequence 4775 | AF038404 | GENBANK |
| Sequence 4776 | AF041483 | GENBANK |
| Sequence 4777 | AF045167 | GENBANK |
| Sequence 4778 | AF047599 | GENBANK |
| Sequence 4779 | AF052187 | GENBANK |
| Sequence 4780 | AF054990 | GENBANK |
| Sequence 4781 | AF060515 | GENBANK |
| Sequence 4782 | AF061258 | GENBANK |
| Sequence 4783 | AF064769 | GENBANK |
| Sequence 4784 | AF070523 | GENBANK |
| Sequence 4785 | AF070561 | GENBANK |
| Sequence 4786 | AF070651 | GENBANK |
| Sequence 4787 | AF070657 | GENBANK |
| Sequence 4788 | AF070665 | GENBANK |
| Sequence 4789 | AF071202 | GENBANK |
| Sequence 4790 | AF074016 | GENBANK |
| Sequence 4791 | AF077030 | GENBANK |
| Sequence 4792 | AF078858 | GENBANK |
| Sequence 4793 | AF083107 | GENBANK |
| Sequence 4794 | AF084555 | GENBANK |
| Sequence 4795 | AF085243 | GENBANK |
| Sequence 4796 | AF085858 | GENBANK |
| Sequence 4797 | AF089747 | GENBANK |
| Sequence 4798 | AF091083 | GENBANK |
| Sequence 4799 | AF092038 | GENBANK |
| Sequence 4800 | AF104252 | GENBANK |
| Sequence 4801 | AF114816 | GENBANK |
| Sequence 4802 | AF124440 | GENBANK |
| Sequence 4803 | AF131775 | GENBANK |
| Sequence 4804 | AF131802 | GENBANK |
| Sequence 4805 | AF132956 | GENBANK |
| Sequence 4806 | AF136450 | GENBANK |
| Sequence 4807 | AF145316 | GENBANK |
| Sequence 4808 | AF147331 | GENBANK |
| Sequence 4809 | AF151840 | GENBANK |
| Sequence 4810 | AF151844 | GENBANK |
| Sequence 4811 | AF151870 | GENBANK |
| Sequence 4812 | AF151902 | GENBANK |
| Sequence 4813 | AF151906 | GENBANK |
| Sequence 4814 | AF157028 | GENBANK |
| Sequence 4815 | AF159056 | GENBANK |
| Sequence 4816 | AF159295 | GENBANK |
| Sequence 4817 | AF168956 | GENBANK |
| Sequence 4818 | AF176555 | GENBANK |
| Sequence 4819 | AF176642 | GENBANK |
| Sequence 4820 | AF182289 | GENBANK |
| Sequence 4821 | AF188745 | GENBANK |
| Sequence 4822 | AF188746 | GENBANK |
| Sequence 4823 | AF191020 | GENBANK |
| Sequence 4824 | AF191339 | GENBANK |
| Sequence 4825 | AJ005259 | GENBANK |
| Sequence 4826 | AJ006470 | GENBANK |
| Sequence 4827 | AJ010482 | GENBANK |
| Sequence 4828 | AJ132583 | GENBANK |
| Sequence 4829 | AJ224172 | GENBANK |
| Sequence 4830 | AJ243310 | GENBANK |
| Sequence 4831 | AL049471 | GENBANK |
| Sequence 4832 | AL050035 | GENBANK |
| Sequence 4833 | AL050037 | GENBANK |
| Sequence 4834 | AL080135 | GENBANK |
| Sequence 4835 | AL110273 | GENBANK |
| Sequence 4836 | AL117471 | GENBANK |
| Sequence 4837 | D10040 | GENBANK |
| Sequence 4838 | D13866 | GENBANK |
| Sequence 4839 | D14662 | GENBANK |
| Sequence 4840 | D16937 | GENBANK |
| Sequence 4841 | D17260 | GENBANK |
| Sequence 4842 | D17409 | GENBANK |
| Sequence 4843 | D28118 | GENBANK |
| Sequence 4844 | D29012 | GENBANK |
| Sequence 4845 | D31763 | GENBANK |
| Sequence 4846 | D38549 | GENBANK |
| Sequence 4847 | D45370 | GENBANK |
| Sequence 4848 | D49489 | GENBANK |
| Sequence 4849 | D50371 | GENBANK |
| Sequence 4850 | D63476 | GENBANK |
| Sequence 4851 | D63875 | GENBANK |
| Sequence 4852 | D87292 | GENBANK |
| Sequence 4853 | D87438 | GENBANK |
| Sequence 4854 | D87666 | GENBANK |
| Sequence 4855 | D89675 | GENBANK |
| Sequence 4856 | D90373 | GENBANK |
| Sequence 4857 | D90427 | GENBANK |
| Sequence 4858 | D90452 | GENBANK |
| Sequence 4859 | E02628 | GENBANK |
| Sequence 4860 | E06721 | GENBANK |
| Sequence 4861 | J02642 | GENBANK |
| Sequence 4862 | J03802 | GENBANK |
| Sequence 4863 | J04183 | GENBANK |
| Sequence 4864 | J04759 | GENBANK |
| Sequence 4865 | K01911 | GENBANK |
| Sequence 4866 | L05091 | GENBANK |
| Sequence 4867 | L05092 | GENBANK |
| Sequence 4868 | L05093 | GENBANK |
| Sequence 4869 | L08044 | GENBANK |
| Sequence 4870 | L08441 | GENBANK |
| Sequence 4871 | L10376 | GENBANK |
| Sequence 4872 | L10678 | GENBANK |
| Sequence 4873 | L15203 | GENBANK |
| Sequence 4874 | L15702 | GENBANK |
| Sequence 4875 | L19597 | GENBANK |
| Sequence 4876 | L20431 | GENBANK |
| Sequence 4877 | L22009 | GENBANK |
| Sequence 4878 | L25081 | GENBANK |
| Sequence 4879 | L38951 | GENBANK |
| Sequence 4880 | M10119 | GENBANK |
| Sequence 4881 | M10906 | GENBANK |
| Sequence 4882 | M11560 | GENBANK |
| Sequence 4883 | M12125 | GENBANK |
| Sequence 4884 | M12623 | GENBANK |
| Sequence 4885 | M13692 | GENBANK |
| Sequence 4886 | M15395 | GENBANK |
| Sequence 4887 | M15885 | GENBANK |
| Sequence 4888 | M16247 | GENBANK |
| Sequence 4889 | M17885 | GENBANK |
| Sequence 4890 | M17886 | GENBANK |
| Sequence 4891 | M21895 | GENBANK |
| Sequence 4892 | M22538 | GENBANK |
| Sequence 4893 | M22918 | GENBANK |
| Sequence 4894 | M26325 | GENBANK |
| Sequence 4895 | M26663 | GENBANK |
| Sequence 4896 | M26880 | GENBANK |
| Sequence 4897 | M29064 | GENBANK |
| Sequence 4898 | M33308 | GENBANK |
| Sequence 4899 | M34840 | GENBANK |
| Sequence 4900 | M36647 | GENBANK |
| Sequence 4901 | M60255 | GENBANK |
| Sequence 4902 | M61832 | GENBANK |
| Sequence 4903 | M64098 | GENBANK |
| Sequence 4904 | M64241 | GENBANK |
| Sequence 4905 | M64571 | GENBANK |
| Sequence 4906 | M68840 | GENBANK |
| Sequence 4907 | M81757 | GENBANK |
| Sequence 4908 | M94345 | GENBANK |
| Sequence 4909 | S50015 | GENBANK |
| Sequence 4910 | S73591 | GENBANK |
| Sequence 4911 | S74678 | GENBANK |
| Sequence 4912 | S75755 | GENBANK |
| Sequence 4913 | U02570 | GENBANK |
| Sequence 4914 | U02619 | GENBANK |
| Sequence 4915 | U07643 | GENBANK |
| Sequence 4916 | U10248 | GENBANK |
| Sequence 4917 | U10439 | GENBANK |
| Sequence 4918 | U14968 | GENBANK |
| Sequence 4919 | U14970 | GENBANK |
| Sequence 4920 | U15174 | GENBANK |
| Sequence 4921 | U16738 | GENBANK |
| Sequence 4922 | U18197 | GENBANK |
| Sequence 4923 | U21049 | GENBANK |
| Sequence 4924 | U22233 | GENBANK |
| Sequence 4925 | U25766 | GENBANK |
| Sequence 4926 | U34038 | GENBANK |
| Sequence 4927 | U36336 | GENBANK |
| Sequence 4928 | U40272 | GENBANK |
| Sequence 4929 | U42068 | GENBANK |
| Sequence 4930 | U49957 | GENBANK |
| Sequence 4931 | U50733 | GENBANK |
| Sequence 4932 | U52962 | GENBANK |
| Sequence 4933 | U52969 | GENBANK |
| Sequence 4934 | U67171 | GENBANK |
| Sequence 4935 | U70063 | GENBANK |
| Sequence 4936 | U79254 | GENBANK |
| Sequence 4937 | U79457 | GENBANK |
| Sequence 4938 | U82226 | GENBANK |
| Sequence 4939 | U82256 | GENBANK |
| Sequence 4940 | U94586 | GENBANK |
| Sequence 4941 | X01037 | GENBANK |
| Sequence 4942 | X01630 | GENBANK |
| Sequence 4943 | X04098 | GENBANK |
| Sequence 4944 | X04106 | GENBANK |
| Sequence 4945 | X04408 | GENBANK |
| Sequence 4946 | X04526 | GENBANK |
| Sequence 4947 | X13839 | GENBANK |
| Sequence 4948 | X15729 | GENBANK |
| Sequence 4949 | X57352 | GENBANK |
| Sequence 4950 | X58141 | GENBANK |
| Sequence 4951 | X60036 | GENBANK |
| Sequence 4952 | X63380 | GENBANK |
| Sequence 4953 | X63422 | GENBANK |
| Sequence 4954 | X63564 | GENBANK |
| Sequence 4955 | X68277 | GENBANK |
| Sequence 4956 | X69711 | GENBANK |
| Sequence 4957 | X73874 | GENBANK |
| Sequence 4958 | X76302 | GENBANK |
| Sequence 4959 | X76732 | GENBANK |
| Sequence 4960 | X81889 | GENBANK |
| Sequence 4961 | X84908 | GENBANK |
| Sequence 4962 | Y00815 | GENBANK |
| Sequence 4963 | Y10351 | GENBANK |
| Sequence 4964 | AA010986 | dbEST |
| Sequence 4965 | AA011203 | dbEST |
| Sequence 4966 | AA026630 | dbEST |
| Sequence 4967 | AA029143 | dbEST |
| Sequence 4968 | AA033869 | dbEST |
| Sequence 4969 | AA034436 | dbEST |
| Sequence 4970 | AA037190 | dbEST |
| Sequence 4971 | AA040127 | dbEST |
| Sequence 4972 | AA043951 | dbEST |
| Sequence 4973 | AA044659 | dbEST |
| Sequence 4974 | AA056603 | dbEST |
| Sequence 4975 | AA057095 | dbEST |
| Sequence 4976 | AA063476 | dbEST |
| Sequence 4977 | AA064886 | dbEST |
| Sequence 4978 | AA071511 | dbEST |
| Sequence 4979 | AA080889 | dbEST |
| Sequence 4980 | AA099520 | dbEST |
| Sequence 4981 | AA099631 | dbEST |
| Sequence 4982 | AA101270 | dbEST |
| Sequence 4983 | AA101674 | dbEST |
| Sequence 4984 | AA127185 | dbEST |
| Sequence 4985 | AA132342 | dbEST |
| Sequence 4986 | AA133798 | dbEST |
| Sequence 4987 | AA150891 | dbEST |
| Sequence 4988 | AA172239 | dbEST |
| Sequence 4989 | AA173348 | dbEST |
| Sequence 4990 | AA173948 | dbEST |
| Sequence 4991 | AA178907 | dbEST |
| Sequence 4992 | AA179409 | dbEST |
| Sequence 4993 | AA190890 | dbEST |
| Sequence 4994 | AA191092 | dbEST |
| Sequence 4995 | AA191476 | dbEST |
| Sequence 4996 | AA195734 | dbEST |
| Sequence 4997 | AA224751 | dbEST |
| Sequence 4998 | AA225488 | dbEST |
| Sequence 4999 | AA225517 | dbEST |
| Sequence 5000 | AA226735 | dbEST |
| Sequence 5001 | AA228953 | dbEST |
| Sequence 5002 | AA229225 | dbEST |
| Sequence 5003 | AA251182 | dbEST |
| Sequence 5004 | AA256401 | dbEST |
| Sequence 5005 | AA258916 | dbEST |
| Sequence 5006 | AA259025 | dbEST |
| Sequence 5007 | AA279054 | dbEST |
| Sequence 5008 | AA292866 | dbEST |
| Sequence 5009 | AA293885 | dbEST |
| Sequence 5010 | AA296534 | dbEST |
| Sequence 5011 | AA296690 | dbEST |
| Sequence 5012 | AA296846 | dbEST |
| Sequence 5013 | AA297270 | dbEST |
| Sequence 5014 | AA298593 | dbEST |
| Sequence 5015 | AA299209 | dbEST |
| Sequence 5016 | AA300065 | dbEST |
| Sequence 5017 | AA301096 | dbEST |
| Sequence 5018 | AA301666 | dbEST |
| Sequence 5019 | AA302919 | dbEST |
| Sequence 5020 | AA303594 | dbEST |
| Sequence 5021 | AA305409 | dbEST |
| Sequence 5022 | AA306378 | dbEST |
| Sequence 5023 | AA310441 | dbEST |
| Sequence 5024 | AA310561 | dbEST |
| Sequence 5025 | AA312444 | dbEST |
| Sequence 5026 | AA313200 | dbEST |
| Sequence 5027 | AA313708 | dbEST |
| Sequence 5028 | AA314188 | dbEST |
| Sequence 5029 | AA314757 | dbEST |
| Sequence 5030 | AA316199 | dbEST |
| Sequence 5031 | AA318544 | dbEST |
| Sequence 5032 | AA320701 | dbEST |
| Sequence 5033 | AA324251 | dbEST |
| Sequence 5034 | AA325214 | dbEST |
| Sequence 5035 | AA327546 | dbEST |
| Sequence 5036 | AA333526 | dbEST |
| Sequence 5037 | AA339065 | dbEST |
| Sequence 5038 | AA340719 | dbEST |
| Sequence 5039 | AA342969 | dbEST |
| Sequence 5040 | AA344067 | dbEST |
| Sequence 5041 | AA346600 | dbEST |
| Sequence 5042 | AA354527 | dbEST |
| Sequence 5043 | AA359763 | dbEST |
| Sequence 5044 | AA364498 | dbEST |
| Sequence 5045 | AA364667 | dbEST |
| Sequence 5046 | AA366518 | dbEST |
| Sequence 5047 | AA371017 | dbEST |
| Sequence 5048 | AA371205 | dbEST |
| Sequence 5049 | AA377218 | dbEST |
| Sequence 5050 | AA384125 | dbEST |
| Sequence 5051 | AA400213 | dbEST |
| Sequence 5052 | AA426235 | dbEST |
| Sequence 5053 | AA430597 | dbEST |
| Sequence 5054 | AA442615 | dbEST |
| Sequence 5055 | AA443184 | dbEST |
| Sequence 5056 | AA443463 | dbEST |
| Sequence 5057 | AA446877 | dbEST |
| Sequence 5058 | AA447700 | dbEST |
| Sequence 5059 | AA448617 | dbEST |
| Sequence 5060 | AA451733 | dbEST |
| Sequence 5061 | AA451988 | dbEST |
| Sequence 5062 | AA454962 | dbEST |
| Sequence 5063 | AA470833 | dbEST |
| Sequence 5064 | AA476509 | dbEST |
| Sequence 5065 | AA483887 | dbEST |
| Sequence 5066 | AA484751 | dbEST |
| Sequence 5067 | AA485767 | dbEST |
| Sequence 5068 | AA486551 | dbEST |
| Sequence 5069 | AA487556 | dbEST |
| Sequence 5070 | AA488567 | dbEST |
| Sequence 5071 | AA489498 | dbEST |
| Sequence 5072 | AA490893 | dbEST |
| Sequence 5073 | AA503943 | dbEST |
| Sequence 5074 | AA507306 | dbEST |
| Sequence 5075 | AA507878 | dbEST |
| Sequence 5076 | AA508861 | dbEST |
| Sequence 5077 | AA523982 | dbEST |
| Sequence 5078 | AA528123 | dbEST |
| Sequence 5079 | AA533716 | dbEST |
| Sequence 5080 | AA534419 | dbEST |
| Sequence 5081 | AA535185 | dbEST |
| Sequence 5082 | AA535695 | dbEST |
| Sequence 5083 | AA541677 | dbEST |
| Sequence 5084 | AA548238 | dbEST |
| Sequence 5085 | AA554735 | dbEST |
| Sequence 5086 | AA570748 | dbEST |
| Sequence 5087 | AA573559 | dbEST |
| Sequence 5088 | AA573811 | dbEST |
| Sequence 5089 | AA578265 | dbEST |
| Sequence 5090 | AA579412 | dbEST |
| Sequence 5091 | AA579998 | dbEST |
| Sequence 5092 | AA588828 | dbEST |
| Sequence 5093 | AA599229 | dbEST |
| Sequence 5094 | AA601289 | dbEST |
| Sequence 5095 | AA633347 | dbEST |
| Sequence 5096 | AA633399 | dbEST |
| Sequence 5097 | AA634196 | dbEST |
| Sequence 5098 | AA669823 | dbEST |
| Sequence 5099 | AA703004 | dbEST |
| Sequence 5100 | AA740574 | dbEST |
| Sequence 5101 | AA743621 | dbEST |
| Sequence 5102 | AA775824 | dbEST |
| Sequence 5103 | AA804375 | dbEST |
| Sequence 5104 | AA810340 | dbEST |
| Sequence 5105 | AA826244 | dbEST |
| Sequence 5106 | AA862855 | dbEST |
| Sequence 5107 | AA906582 | dbEST |
| Sequence 5108 | AA908797 | dbEST |
| Sequence 5109 | AA934755 | dbEST |
| Sequence 5110 | AA974402 | dbEST |
| Sequence 5111 | AA984460 | dbEST |
| Sequence 5112 | AA988102 | dbEST |
| Sequence 5113 | AA992672 | dbEST |
| Sequence 5114 | AI041150 | dbEST |
| Sequence 5115 | AI042406 | dbEST |
| Sequence 5116 | AI057617 | dbEST |
| Sequence 5117 | AI065152 | dbEST |
| Sequence 5118 | AI074232 | dbEST |
| Sequence 5119 | AI081133 | dbEST |
| Sequence 5120 | AI096706 | dbEST |
| Sequence 5121 | AI110765 | dbEST |
| Sequence 5122 | AI125166 | dbEST |
| Sequence 5123 | AI126653 | dbEST |
| Sequence 5124 | AI128091 | dbEST |
| Sequence 5125 | AI133668 | dbEST |
| Sequence 5126 | AI140793 | dbEST |
| Sequence 5127 | AI143746 | dbEST |
| Sequence 5128 | AI144423 | dbEST |
| Sequence 5129 | AI186597 | dbEST |
| Sequence 5130 | AI188794 | dbEST |
| Sequence 5131 | AI189345 | dbEST |
| Sequence 5132 | AI198311 | dbEST |
| Sequence 5133 | AI199681 | dbEST |
| Sequence 5134 | AI214048 | dbEST |
| Sequence 5135 | AI216969 | dbEST |
| Sequence 5136 | AI243841 | dbEST |
| Sequence 5137 | AI246497 | dbEST |
| Sequence 5138 | AI253395 | dbEST |
| Sequence 5139 | AI267158 | dbEST |
| Sequence 5140 | AI267216 | dbEST |
| Sequence 5141 | AI267282 | dbEST |
| Sequence 5142 | AI267502 | dbEST |
| Sequence 5143 | AI268362 | dbEST |
| Sequence 5144 | AI275114 | dbEST |
| Sequence 5145 | AI277434 | dbEST |
| Sequence 5146 | AI281946 | dbEST |
| Sequence 5147 | AI284667 | dbEST |
| Sequence 5148 | AI288388 | dbEST |
| Sequence 5149 | AI289278 | dbEST |
| Sequence 5150 | AI307634 | dbEST |
| Sequence 5151 | AI310235 | dbEST |
| Sequence 5152 | AI360451 | dbEST |
| Sequence 5153 | AI368664 | dbEST |
| Sequence 5154 | AI373544 | dbEST |
| Sequence 5155 | AI375919 | dbEST |
| Sequence 5156 | AI457216 | dbEST |
| Sequence 5157 | AI459667 | dbEST |
| Sequence 5158 | AI473779 | dbEST |
| Sequence 5159 | AI525843 | dbEST |
| Sequence 5160 | AI536685 | dbEST |
| Sequence 5161 | AI547284 | dbEST |
| Sequence 5162 | AI557458 | dbEST |
| Sequence 5163 | AI570141 | dbEST |
| Sequence 5164 | AI587642 | dbEST |
| Sequence 5165 | AI632523 | dbEST |
| Sequence 5166 | AI640588 | dbEST |
| Sequence 5167 | AI651069 | dbEST |
| Sequence 5168 | AI660243 | dbEST |
| Sequence 5169 | AI677810 | dbEST |
| Sequence 5170 | AI685632 | dbEST |
| Sequence 5171 | AI686957 | dbEST |
| Sequence 5172 | AI694572 | dbEST |
| Sequence 5173 | AI708884 | dbEST |
| Sequence 5174 | AI708995 | dbEST |
| Sequence 5175 | AI749547 | dbEST |
| Sequence 5176 | AI749886 | dbEST |
| Sequence 5177 | AI815664 | dbEST |
| Sequence 5178 | AI815966 | dbEST |
| Sequence 5179 | AI860016 | dbEST |
| Sequence 5180 | AI878918 | dbEST |
| Sequence 5181 | AI885174 | dbEST |
| Sequence 5182 | AI888493 | dbEST |
| Sequence 5183 | AI929664 | dbEST |
| Sequence 5184 | AI936472 | dbEST |
| Sequence 5185 | AI955087 | dbEST |
| Sequence 5186 | AI991469 | dbEST |
| Sequence 5187 | AL035985 | dbEST |
| Sequence 5188 | AL037724 | dbEST |
| Sequence 5189 | AL042536 | dbEST |
| Sequence 5190 | AW003287 | dbEST |
| Sequence 5191 | AW024797 | dbEST |
| Sequence 5192 | AW071906 | dbEST |
| Sequence 5193 | AW130325 | dbEST |
| Sequence 5194 | AW152107 | dbEST |
| Sequence 5195 | AW177929 | dbEST |
| Sequence 5196 | AW177931 | dbEST |
| Sequence 5197 | C05816 | dbEST |
| Sequence 5198 | C18484 | dbEST |
| Sequence 5199 | D58694 | dbEST |
| Sequence 5200 | F24428 | dbEST |
| Sequence 5201 | F27796 | dbEST |
| Sequence 5202 | H00112 | dbEST |
| Sequence 5203 | H08924 | dbEST |
| Sequence 5204 | H18732 | dbEST |
| Sequence 5205 | H58534 | dbEST |
| Sequence 5206 | M85357 | dbEST |
| Sequence 5207 | N40017 | dbEST |
| Sequence 5208 | N86292 | dbEST |
| Sequence 5209 | R70270 | dbEST |
| Sequence 5210 | R99478 | dbEST |
| Sequence 5211 | W00471 | dbEST |
| Sequence 5212 | W04380 | dbEST |
| Sequence 5213 | W07165 | dbEST |
| Sequence 5214 | W37945 | dbEST |
| Sequence 5215 | W73588 | dbEST |
| Sequence 5216 | W78967 | dbEST |
| Sequence 5217 | W85764 | dbEST |
| Sequence 5218 | N60172 | NUCPATENT |
| Sequence 5219 | N91825 | NUCPATENT |
| Sequence 5220 | Q43526 | NUCPATENT |
| Sequence 5221 | Q66256 | NUCPATENT |
| Sequence 5222 | Q74061 | NUCPATENT |
| Sequence 5223 | T00880 | NUCPATENT |
| Sequence 5224 | T04864 | NUCPATENT |
| Sequence 5225 | T36781 | NUCPATENT |
| Sequence 5226 | T59998 | NUCPATENT |
| Sequence 5227 | T89060 | NUCPATENT |
| Sequence 5228 | T91054 | NUCPATENT |
| Sequence 5229 | V07571 | NUCPATENT |
| Sequence 5230 | V34212 | NUCPATENT |
| Sequence 5231 | V45442 | NUCPATENT |
| Sequence 5232 | V64828 | NUCPATENT |
| Sequence 5233 | V69855 | NUCPATENT |
| Sequence 5234 | W71075 | NUCPATENT |
| Sequence 5235 | V71742 | NUCPATENT |
| Sequence 5236 | V74071 | NUCPATENT |
| Sequence 5237 | V84395 | NUCPATENT |
| Sequence 5238 | V86225 | NUCPATENT |
| Sequence 5239 | V86232 | NUCPATENT |
| Sequence 5240 | X04125 | NUCPATENT |
| Sequence 5241 | X87624 | NUCPATENT |
| Sequence 5242 | X98561 | NUCPATENT |
| Sequence 5243 | X99098 | NUCPATENT |
| Sequence 5244 | Z06250 | NUCPATENT |
| Sequence 5245 | Z11491 | NUCPATENT |
| Sequence 5246 | Z17710 | NUCPATENT |
| Sequence 5247 | Z33671 | NUCPATENT |
| Sequence 5248 | A06846 | GENBANK |
| Sequence 5249 | A06919 | GENBANK |
| Sequence 5250 | A06925 | GENBANK |
| Sequence 5251 | A21185 | GENBANK |
| Sequence 5252 | A23013 | GENBANK |
| Sequence 5253 | AB000220 | GENBANK |
| Sequence 5254 | AB000584 | GENBANK |
| Sequence 5255 | AB002312 | GENBANK |
| Sequence 5256 | AB002350 | GENBANK |
| Sequence 5257 | AB002382 | GENBANK |
| Sequence 5258 | AB002806 | GENBANK |
| Sequence 5259 | AB004851 | GENBANK |
| Sequence 5260 | AB005535 | GENBANK |
| Sequence 5261 | AB006534 | GENBANK |
| Sequence 5262 | AB006621 | GENBANK |
| Sequence 5263 | AB007867 | GENBANK |
| Sequence 5264 | AB007882 | GENBANK |
| Sequence 5265 | AB007892 | GENBANK |
| Sequence 5266 | AB007931 | GENBANK |
| Sequence 5267 | AB007947 | GENBANK |
| Sequence 5268 | AB008226 | GENBANK |
| Sequence 5269 | AB011164 | GENBANK |
| Sequence 5270 | AB014511 | GENBANK |
| Sequence 5271 | AB014542 | GENBANK |
| Sequence 5272 | AB014552 | GENBANK |
| Sequence 5273 | AB018274 | GENBANK |
| Sequence 5274 | AB018284 | GENBANK |
| Sequence 5275 | AB018301 | GENBANK |
| Sequence 5276 | AB018315 | GENBANK |
| Sequence 5277 | AB018319 | GENBANK |
| Sequence 5278 | AB018340 | GENBANK |
| Sequence 5279 | AB018352 | GENBANK |
| Sequence 5280 | AB019568 | GENBANK |
| Sequence 5281 | AB019691 | GENBANK |
| Sequence 5282 | AB020637 | GENBANK |
| Sequence 5283 | AB020652 | GENBANK |
| Sequence 5284 | AB020692 | GENBANK |
| Sequence 5285 | AB020698 | GENBANK |
| Sequence 5286 | AB021288 | GENBANK |
| Sequence 5287 | AB023150 | GENBANK |
| Sequence 5288 | AB023153 | GENBANK |
| Sequence 5289 | AB027466 | GENBANK |
| Sequence 5290 | AB028964 | GENBANK |
| Sequence 5291 | AB028971 | GENBANK |
| Sequence 5292 | AB029000 | GENBANK |
| Sequence 5293 | AB029027 | GENBANK |
| Sequence 5294 | AB032957 | GENBANK |
| Sequence 5295 | AB033010 | GENBANK |
| Sequence 5296 | AB033017 | GENBANK |
| Sequence 5297 | AB033070 | GENBANK |
| Sequence 5298 | AB033074 | GENBANK |
| Sequence 5299 | AF007791 | GENBANK |
| Sequence 5300 | AF008443 | GENBANK |
| Sequence 5301 | AF013759 | GENBANK |
| Sequence 5302 | AF015283 | GENBANK |
| Sequence 5303 | AF017305 | GENBANK |
| Sequence 5304 | AF017445 | GENBANK |
| Sequence 5305 | AF019226 | GENBANK |
| Sequence 5306 | AF020038 | GENBANK |
| Sequence 5307 | AF020591 | GENBANK |
| Sequence 5308 | AF020768 | GENBANK |
| Sequence 5309 | AF021336 | GENBANK |
| Sequence 5310 | AF023676 | GENBANK |
| Sequence 5311 | AF025998 | GENBANK |
| Sequence 5312 | AF026292 | GENBANK |
| Sequence 5313 | AF027302 | GENBANK |
| Sequence 5314 | AF034209 | GENBANK |
| Sequence 5315 | AF034795 | GENBANK |
| Sequence 5316 | AF038172 | GENBANK |
| Sequence 5317 | AF038175 | GENBANK |
| Sequence 5318 | AF038404 | GENBANK |
| Sequence 5319 | AF038451 | GENBANK |
| Sequence 5320 | AF038962 | GENBANK |
| Sequence 5321 | AF039081 | GENBANK |
| Sequence 5322 | AF039918 | GENBANK |
| Sequence 5323 | AF040250 | GENBANK |
| Sequence 5324 | AF040958 | GENBANK |
| Sequence 5325 | AF042166 | GENBANK |
| Sequence 5326 | AF042284 | GENBANK |
| Sequence 5327 | AF042384 | GENBANK |
| Sequence 5328 | AF045583 | GENBANK |
| Sequence 5329 | AF047020 | GENBANK |
| Sequence 5330 | AF047185 | GENBANK |
| Sequence 5331 | AF047436 | GENBANK |
| Sequence 5332 | AF048731 | GENBANK |
| Sequence 5333 | AF050641 | GENBANK |
| Sequence 5334 | AF051321 | GENBANK |
| Sequence 5335 | AF052093 | GENBANK |
| Sequence 5336 | AF052149 | GENBANK |
| Sequence 5337 | AF052175 | GENBANK |
| Sequence 5338 | AF053070 | GENBANK |
| Sequence 5339 | AF054175 | GENBANK |
| Sequence 5340 | AF054181 | GENBANK |
| Sequence 5341 | AF054838 | GENBANK |
| Sequence 5342 | AF054988 | GENBANK |
| Sequence 5343 | AF057706 | GENBANK |
| Sequence 5344 | AF058718 | GENBANK |
| Sequence 5345 | AF060219 | GENBANK |
| Sequence 5346 | AF061258 | GENBANK |
| Sequence 5347 | AF062318 | GENBANK |
| Sequence 5348 | AF064491 | GENBANK |
| Sequence 5349 | AF064819 | GENBANK |
| Sequence 5350 | AF065388 | GENBANK |
| Sequence 5351 | AF068754 | GENBANK |
| Sequence 5352 | AF069073 | GENBANK |
| Sequence 5353 | AF069601 | GENBANK |
| Sequence 5354 | AF070523 | GENBANK |
| Sequence 5355 | AF070548 | GENBANK |
| Sequence 5356 | AF070553 | GENBANK |
| Sequence 5357 | AF070554 | GENBANK |
| Sequence 5358 | AF070562 | GENBANK |
| Sequence 5359 | AF070596 | GENBANK |
| Sequence 5360 | AF070652 | GENBANK |
| Sequence 5361 | AF071202 | GENBANK |
| Sequence 5362 | AF072371 | GENBANK |
| Sequence 5363 | AF077030 | GENBANK |
| Sequence 5364 | AF077037 | GENBANK |
| Sequence 5365 | AF077202 | GENBANK |
| Sequence 5366 | AF078854 | GENBANK |
| Sequence 5367 | AF078858 | GENBANK |
| Sequence 5368 | AF078863 | GENBANK |
| Sequence 5369 | AF082858 | GENBANK |
| Sequence 5370 | AF082889 | GENBANK |
| Sequence 5371 | AF083190 | GENBANK |
| Sequence 5372 | AF086168 | GENBANK |
| Sequence 5373 | AF086245 | GENBANK |
| Sequence 5374 | AF086336 | GENBANK |
| Sequence 5375 | AF087020 | GENBANK |
| Sequence 5376 | AF089747 | GENBANK |
| Sequence 5377 | AF091084 | GENBANK |
| Sequence 5378 | AF092135 | GENBANK |
| Sequence 5379 | AF096773 | GENBANK |
| Sequence 5380 | AF096774 | GENBANK |
| Sequence 5381 | AF097514 | GENBANK |
| Sequence 5382 | AF099149 | GENBANK |
| Sequence 5383 | AF100741 | GENBANK |
| Sequence 5384 | AF100756 | GENBANK |
| Sequence 5385 | AF102846 | GENBANK |
| Sequence 5386 | AF103907 | GENBANK |
| Sequence 5387 | AF112227 | GENBANK |
| Sequence 5388 | AF126181 | GENBANK |
| Sequence 5389 | AF128528 | GENBANK |
| Sequence 5390 | AF131220 | GENBANK |
| Sequence 5391 | AF132940 | GENBANK |
| Sequence 5392 | AF138300 | GENBANK |
| Sequence 5393 | AF147331 | GENBANK |
| Sequence 5394 | AF147334 | GENBANK |
| Sequence 5395 | AF149045 | GENBANK |
| Sequence 5396 | AF151105 | GENBANK |
| Sequence 5397 | AF151800 | GENBANK |
| Sequence 5398 | AF151840 | GENBANK |
| Sequence 5399 | AF151846 | GENBANK |
| Sequence 5400 | AF151861 | GENBANK |
| Sequence 5401 | AF151868 | GENBANK |
| Sequence 5402 | AF151884 | GENBANK |
| Sequence 5403 | AF151893 | GENBANK |
| Sequence 5404 | AF159056 | GENBANK |
| Sequence 5405 | AF159092 | GENBANK |
| Sequence 5406 | AF159295 | GENBANK |
| Sequence 5407 | AF168956 | GENBANK |
| Sequence 5408 | AF177291 | GENBANK |
| Sequence 5409 | AF177775 | GENBANK |
| Sequence 5410 | AF188298 | GENBANK |
| Sequence 5411 | AF188745 | GENBANK |
| Sequence 5412 | AF188746 | GENBANK |
| Sequence 5413 | AF190167 | GENBANK |
| Sequence 5414 | AJ010046 | GENBANK |
| Sequence 5415 | AJ010482 | GENBANK |
| Sequence 5416 | AJ012463 | GENBANK |
| Sequence 5417 | AJ012499 | GENBANK |
| Sequence 5418 | AJ130733 | GENBANK |
| Sequence 5419 | AJ223075 | GENBANK |
| Sequence 5420 | AJ223350 | GENBANK |
| Sequence 5421 | AJ224172 | GENBANK |
| Sequence 5422 | AJ249731 | GENBANK |
| Sequence 5423 | AL049705 | GENBANK |
| Sequence 5424 | AL049969 | GENBANK |
| Sequence 5425 | AL050198 | GENBANK |
| Sequence 5426 | AL050274 | GENBANK |
| Sequence 5427 | AL080089 | GENBANK |
| Sequence 5428 | AL080135 | GENBANK |
| Sequence 5429 | AL080209 | GENBANK |
| Sequence 5430 | AL096880 | GENBANK |
| Sequence 5431 | AL109672 | GENBANK |
| Sequence 5432 | AL110183 | GENBANK |
| Sequence 5433 | AL117413 | GENBANK |
| Sequence 5434 | AL117550 | GENBANK |
| Sequence 5435 | AL117595 | GENBANK |
| Sequence 5436 | AL121733 | GENBANK |
| Sequence 5437 | D00422 | GENBANK |
| Sequence 5438 | D13641 | GENBANK |
| Sequence 5439 | D13866 | GENBANK |
| Sequence 5440 | D13900 | GENBANK |
| Sequence 5441 | D14662 | GENBANK |
| Sequence 5442 | D14664 | GENBANK |
| Sequence 5443 | D16111 | GENBANK |
| Sequence 5444 | D16922 | GENBANK |
| Sequence 5445 | D17409 | GENBANK |
| Sequence 5446 | D17554 | GENBANK |
| Sequence 5447 | D21243 | GENBANK |
| Sequence 5448 | D21851 | GENBANK |
| Sequence 5449 | D21853 | GENBANK |
| Sequence 5450 | D26068 | GENBANK |
| Sequence 5451 | D28482 | GENBANK |
| Sequence 5452 | D29640 | GENBANK |
| Sequence 5453 | D29954 | GENBANK |
| Sequence 5454 | D31886 | GENBANK |
| Sequence 5455 | D38037 | GENBANK |
| Sequence 5456 | D38549 | GENBANK |
| Sequence 5457 | D42039 | GENBANK |
| Sequence 5458 | D42044 | GENBANK |
| Sequence 5459 | D42047 | GENBANK |
| Sequence 5460 | D42138 | GENBANK |
| Sequence 5461 | D43950 | GENBANK |
| Sequence 5462 | D49372 | GENBANK |
| Sequence 5463 | D49489 | GENBANK |
| Sequence 5464 | D50369 | GENBANK |
| Sequence 5465 | D50371 | GENBANK |
| Sequence 5466 | D50579 | GENBANK |
| Sequence 5467 | D50916 | GENBANK |
| Sequence 5468 | D50919 | GENBANK |
| Sequence 5469 | D55653 | GENBANK |
| Sequence 5470 | D63486 | GENBANK |
| Sequence 5471 | D64110 | GENBANK |
| Sequence 5472 | D80009 | GENBANK |
| Sequence 5473 | D82060 | GENBANK |
| Sequence 5474 | D83032 | GENBANK |
| Sequence 5475 | D83735 | GENBANK |
| Sequence 5476 | D85181 | GENBANK |
| Sequence 5477 | D86984 | GENBANK |
| Sequence 5478 | D87127 | GENBANK |
| Sequence 5479 | D87292 | GENBANK |
| Sequence 5480 | D87453 | GENBANK |
| Sequence 5481 | D87462 | GENBANK |
| Sequence 5482 | D87666 | GENBANK |
| Sequence 5483 | D87684 | GENBANK |
| Sequence 5484 | D87735 | GENBANK |
| Sequence 5485 | D87969 | GENBANK |
| Sequence 5486 | D88674 | GENBANK |
| Sequence 5487 | D90373 | GENBANK |
| Sequence 5488 | D90427 | GENBANK |
| Sequence 5489 | E01650 | GENBANK |
| Sequence 5490 | E01915 | GENBANK |
| Sequence 5491 | E02628 | GENBANK |
| Sequence 5492 | E03413 | GENBANK |
| Sequence 5493 | E06721 | GENBANK |
| Sequence 5494 | J00194 | GENBANK |
| Sequence 5495 | J02642 | GENBANK |
| Sequence 5496 | J02876 | GENBANK |
| Sequence 5497 | J02908 | GENBANK |
| Sequence 5498 | J03007 | GENBANK |
| Sequence 5499 | J03015 | GENBANK |
| Sequence 5500 | J03171 | GENBANK |
| Sequence 5501 | J03248 | GENBANK |
| Sequence 5502 | J03507 | GENBANK |
| Sequence 5503 | J03592 | GENBANK |
| Sequence 5504 | J03799 | GENBANK |
| Sequence 5505 | J03870 | GENBANK |
| Sequence 5506 | J05633 | GENBANK |
| Sequence 5507 | K00409 | GENBANK |
| Sequence 5508 | K01911 | GENBANK |
| Sequence 5509 | L05092 | GENBANK |
| Sequence 5510 | L05093 | GENBANK |
| Sequence 5511 | L05779 | GENBANK |
| Sequence 5512 | L07033 | GENBANK |
| Sequence 5513 | L07517 | GENBANK |
| Sequence 5514 | L08044 | GENBANK |
| Sequence 5515 | L12136 | GENBANK |
| Sequence 5516 | L15203 | GENBANK |
| Sequence 5517 | L16785 | GENBANK |
| Sequence 5518 | L19597 | GENBANK |
| Sequence 5519 | L19605 | GENBANK |
| Sequence 5520 | L19956 | GENBANK |
| Sequence 5521 | L20773 | GENBANK |
| Sequence 5522 | L20941 | GENBANK |
| Sequence 5523 | L23320 | GENBANK |
| Sequence 5524 | L28010 | GENBANK |
| Sequence 5525 | L29008 | GENBANK |
| Sequence 5526 | L31951 | GENBANK |
| Sequence 5527 | L34840 | GENBANK |
| Sequence 5528 | L37080 | GENBANK |
| Sequence 5529 | L38486 | GENBANK |
| Sequence 5530 | L38961 | GENBANK |
| Sequence 5531 | L38995 | GENBANK |
| Sequence 5532 | L42531 | GENBANK |
| Sequence 5533 | L47665 | GENBANK |
| Sequence 5534 | M10036 | GENBANK |
| Sequence 5535 | M10119 | GENBANK |
| Sequence 5536 | M13520 | GENBANK |
| Sequence 5537 | M14335 | GENBANK |
| Sequence 5538 | M15470 | GENBANK |
| Sequence 5539 | M15887 | GENBANK |
| Sequence 5540 | M16660 | GENBANK |
| Sequence 5541 | M16804 | GENBANK |
| Sequence 5542 | M17851 | GENBANK |
| Sequence 5543 | M19308 | GENBANK |
| Sequence 5544 | M20471 | GENBANK |
| Sequence 5545 | M21895 | GENBANK |
| Sequence 5546 | M21896 | GENBANK |
| Sequence 5547 | M22324 | GENBANK |
| Sequence 5548 | M24194 | GENBANK |
| Sequence 5549 | M24795 | GENBANK |
| Sequence 5550 | M24902 | GENBANK |
| Sequence 5551 | M24906 | GENBANK |
| Sequence 5552 | M25246 | GENBANK |
| Sequence 5553 | M26324 | GENBANK |
| Sequence 5554 | M26325 | GENBANK |
| Sequence 5555 | M26663 | GENBANK |
| Sequence 5556 | M27110 | GENBANK |
| Sequence 5557 | M28016 | GENBANK |
| Sequence 5558 | M28211 | GENBANK |
| Sequence 5559 | M29366 | GENBANK |
| Sequence 5560 | M29872 | GENBANK |
| Sequence 5561 | M31212 | GENBANK |
| Sequence 5562 | M31606 | GENBANK |
| Sequence 5563 | M33146 | GENBANK |
| Sequence 5564 | M33308 | GENBANK |
| Sequence 5565 | M34840 | GENBANK |
| Sequence 5566 | M36634 | GENBANK |
| Sequence 5567 | M57399 | GENBANK |
| Sequence 5568 | M57567 | GENBANK |
| Sequence 5569 | M58028 | GENBANK |
| Sequence 5570 | M58485 | GENBANK |
| Sequence 5571 | M58549 | GENBANK |
| Sequence 5572 | M60457 | GENBANK |
| Sequence 5573 | M60857 | GENBANK |
| Sequence 5574 | M61832 | GENBANK |
| Sequence 5575 | M63573 | GENBANK |
| Sequence 5576 | M64098 | GENBANK |
| Sequence 5577 | M64241 | GENBANK |
| Sequence 5578 | M64571 | GENBANK |
| Sequence 5579 | M69066 | GENBANK |
| Sequence 5580 | M69180 | GENBANK |
| Sequence 5581 | M74777 | GENBANK |
| Sequence 5582 | M76180 | GENBANK |
| Sequence 5583 | M77830 | GENBANK |
| Sequence 5584 | M81757 | GENBANK |
| Sequence 5585 | M86752 | GENBANK |
| Sequence 5586 | M88279 | GENBANK |
| Sequence 5587 | M92381 | GENBANK |
| Sequence 5588 | M92843 | GENBANK |
| Sequence 5589 | M95178 | GENBANK |
| Sequence 5590 | M99701 | GENBANK |
| Sequence 5591 | S60099 | GENBANK |
| Sequence 5592 | S67325 | GENBANK |
| Sequence 5593 | S69022 | GENBANK |
| Sequence 5594 | S69272 | GENBANK |
| Sequence 5595 | S69738 | GENBANK |
| Sequence 5596 | S70154 | GENBANK |
| Sequence 5597 | S73591 | GENBANK |
| Sequence 5598 | S74678 | GENBANK |
| Sequence 5599 | S75755 | GENBANK |
| Sequence 5600 | U02390 | GENBANK |
| Sequence 5601 | U04209 | GENBANK |
| Sequence 5602 | U05291 | GENBANK |
| Sequence 5603 | U06632 | GENBANK |
| Sequence 5604 | U07919 | GENBANK |
| Sequence 5605 | U09550 | GENBANK |
| Sequence 5606 | U14510 | GENBANK |
| Sequence 5607 | U14750 | GENBANK |
| Sequence 5608 | U14968 | GENBANK |
| Sequence 5609 | U14969 | GENBANK |
| Sequence 5610 | U14970 | GENBANK |
| Sequence 5611 | U16850 | GENBANK |
| Sequence 5612 | U16997 | GENBANK |
| Sequence 5613 | U17743 | GENBANK |
| Sequence 5614 | U18062 | GENBANK |
| Sequence 5615 | U18197 | GENBANK |
| Sequence 5616 | U18291 | GENBANK |
| Sequence 5617 | U18543 | GENBANK |
| Sequence 5618 | U27143 | GENBANK |
| Sequence 5619 | U28424 | GENBANK |
| Sequence 5620 | U30313 | GENBANK |
| Sequence 5621 | U30826 | GENBANK |
| Sequence 5622 | U31382 | GENBANK |
| Sequence 5623 | U31905 | GENBANK |
| Sequence 5624 | U36764 | GENBANK |
| Sequence 5625 | U37519 | GENBANK |
| Sequence 5626 | U39360 | GENBANK |
| Sequence 5627 | U39400 | GENBANK |
| Sequence 5628 | U40272 | GENBANK |
| Sequence 5629 | U43701 | GENBANK |
| Sequence 5630 | U44839 | GENBANK |
| Sequence 5631 | U46751 | GENBANK |
| Sequence 5632 | U51903 | GENBANK |
| Sequence 5633 | U54562 | GENBANK |
| Sequence 5634 | U54996 | GENBANK |
| Sequence 5635 | U58855 | GENBANK |
| Sequence 5636 | U66615 | GENBANK |
| Sequence 5637 | U67171 | GENBANK |
| Sequence 5638 | U69263 | GENBANK |
| Sequence 5639 | U69668 | GENBANK |
| Sequence 5640 | U79241 | GENBANK |
| Sequence 5641 | U79304 | GENBANK |
| Sequence 5642 | U80456 | GENBANK |
| Sequence 5643 | U83460 | GENBANK |
| Sequence 5644 | U95646 | GENBANK |
| Sequence 5645 | U96721 | GENBANK |
| Sequence 5646 | X00351 | GENBANK |
| Sequence 5647 | X04106 | GENBANK |
| Sequence 5648 | X04325 | GENBANK |
| Sequence 5649 | X04408 | GENBANK |
| Sequence 5650 | X04571 | GENBANK |
| Sequence 5651 | X05332 | GENBANK |
| Sequence 5652 | X06617 | GENBANK |
| Sequence 5653 | X07876 | GENBANK |
| Sequence 5654 | X13839 | GENBANK |
| Sequence 5655 | X14608 | GENBANK |
| Sequence 5656 | X15187 | GENBANK |
| Sequence 5657 | X16940 | GENBANK |
| Sequence 5658 | X17206 | GENBANK |
| Sequence 5659 | X53777 | GENBANK |
| Sequence 5660 | X56998 | GENBANK |
| Sequence 5661 | X57398 | GENBANK |
| Sequence 5662 | X60221 | GENBANK |
| Sequence 5663 | X60708 | GENBANK |
| Sequence 5664 | X66276 | GENBANK |
| Sequence 5665 | X66534 | GENBANK |
| Sequence 5666 | X67951 | GENBANK |
| Sequence 5667 | X70394 | GENBANK |
| Sequence 5668 | X71129 | GENBANK |
| Sequence 5669 | X73114 | GENBANK |
| Sequence 5670 | X74070 | GENBANK |
| Sequence 5671 | X75546 | GENBANK |
| Sequence 5672 | X75593 | GENBANK |
| Sequence 5673 | X75861 | GENBANK |
| Sequence 5674 | X76105 | GENBANK |
| Sequence 5675 | X76538 | GENBANK |
| Sequence 5676 | X77196 | GENBANK |
| Sequence 5677 | X78137 | GENBANK |
| Sequence 5678 | X78520 | GENBANK |
| Sequence 5679 | X79389 | GENBANK |
| Sequence 5680 | X79888 | GENBANK |
| Sequence 5681 | X80822 | GENBANK |
| Sequence 5682 | X82157 | GENBANK |
| Sequence 5683 | X85373 | GENBANK |
| Sequence 5684 | X87241 | GENBANK |
| Sequence 5685 | X90583 | GENBANK |
| Sequence 5686 | X92098 | GENBANK |
| Sequence 5687 | X93036 | GENBANK |
| Sequence 5688 | X97065 | GENBANK |
| Sequence 5689 | X97674 | GENBANK |
| Sequence 5690 | X98801 | GENBANK |
| Sequence 5691 | Y00282 | GENBANK |
| Sequence 5692 | Y07968 | GENBANK |
| Sequence 5693 | Y12781 | GENBANK |
| Sequence 5694 | Y13936 | GENBANK |
| Sequence 5695 | Y16132 | GENBANK |
| Sequence 5696 | Y16610 | GENBANK |
| Sequence 5697 | Z26317 | GENBANK |
| Sequence 5698 | Z46973 | GENBANK |
| Sequence 5699 | Z50749 | GENBANK |
| Sequence 5700 | AA001299 | dbEST |
| Sequence 5701 | AA001316 | dbEST |
| Sequence 5702 | AA002190 | dbEST |
| Sequence 5703 | AA009641 | dbEST |
| Sequence 5704 | AA010236 | dbEST |
| Sequence 5705 | AA010607 | dbEST |
| Sequence 5706 | AA013094 | dbEST |
| Sequence 5707 | AA015605 | dbEST |
| Sequence 5708 | AA018347 | dbEST |
| Sequence 5709 | AA019377 | dbEST |
| Sequence 5710 | AA022583 | dbEST |
| Sequence 5711 | AA024398 | dbEST |
| Sequence 5712 | AA024532 | dbEST |
| Sequence 5713 | AA024967 | dbEST |
| Sequence 5714 | AA025731 | dbEST |
| Sequence 5715 | AA025875 | dbEST |
| Sequence 5716 | AA026658 | dbEST |
| Sequence 5717 | AA029143 | dbEST |
| Sequence 5718 | AA029157 | dbEST |
| Sequence 5719 | AA029584 | dbEST |
| Sequence 5720 | AA033517 | dbEST |
| Sequence 5721 | AA034255 | dbEST |
| Sequence 5722 | AA034946 | dbEST |
| Sequence 5723 | AA035773 | dbEST |
| Sequence 5724 | AA037217 | dbEST |
| Sequence 5725 | AA037857 | dbEST |
| Sequence 5726 | AA039546 | dbEST |
| Sequence 5727 | AA040140 | dbEST |
| Sequence 5728 | AA044004 | dbEST |
| Sequence 5729 | AA044291 | dbEST |
| Sequence 5730 | AA044570 | dbEST |
| Sequence 5731 | AA045185 | dbEST |
| Sequence 5732 | AA045342 | dbEST |
| Sequence 5733 | AA045388 | dbEST |
| Sequence 5734 | AA045741 | dbEST |
| Sequence 5735 | AA046040 | dbEST |
| Sequence 5736 | AA046483 | dbEST |
| Sequence 5737 | AA046836 | dbEST |
| Sequence 5738 | AA046973 | dbEST |
| Sequence 5739 | AA047015 | dbEST |
| Sequence 5740 | AA047304 | dbEST |
| Sequence 5741 | AA053250 | dbEST |
| Sequence 5742 | AA053399 | dbEST |
| Sequence 5743 | AA053475 | dbEST |
| Sequence 5744 | AA053514 | dbEST |
| Sequence 5745 | AA053557 | dbEST |
| Sequence 5746 | AA053747 | dbEST |
| Sequence 5747 | AA053757 | dbEST |
| Sequence 5748 | AA056462 | dbEST |
| Sequence 5749 | AA057029 | dbEST |
| Sequence 5750 | AA057243 | dbEST |
| Sequence 5751 | AA058605 | dbEST |
| Sequence 5752 | AA058899 | dbEST |
| Sequence 5753 | AA059309 | dbEST |
| Sequence 5754 | AA059355 | dbEST |
| Sequence 5755 | AA062994 | dbEST |
| Sequence 5756 | AA065036 | dbEST |
| Sequence 5757 | AA065214 | dbEST |
| Sequence 5758 | AA069457 | dbEST |
| Sequence 5759 | AA069814 | dbEST |
| Sequence 5760 | AA071084 | dbEST |
| Sequence 5761 | AA071439 | dbEST |
| Sequence 5762 | AA074575 | dbEST |
| Sequence 5763 | AA074683 | dbEST |
| Sequence 5764 | AA074819 | dbEST |
| Sequence 5765 | AA075474 | dbEST |
| Sequence 5766 | AA075600 | dbEST |
| Sequence 5767 | AA075781 | dbEST |
| Sequence 5768 | AA075922 | dbEST |
| Sequence 5769 | AA078218 | dbEST |
| Sequence 5770 | AA079346 | dbEST |
| Sequence 5771 | AA079524 | dbEST |
| Sequence 5772 | AA079690 | dbEST |
| Sequence 5773 | AA079755 | dbEST |
| Sequence 5774 | AA081708 | dbEST |
| Sequence 5775 | AA081812 | dbEST |
| Sequence 5776 | AA081842 | dbEST |
| Sequence 5777 | AA082839 | dbEST |
| Sequence 5778 | AA083008 | dbEST |
| Sequence 5779 | AA083191 | dbEST |
| Sequence 5780 | AA083345 | dbEST |
| Sequence 5781 | AA083410 | dbEST |
| Sequence 5782 | AA083603 | dbEST |
| Sequence 5783 | AA084068 | dbEST |
| Sequence 5784 | AA084552 | dbEST |
| Sequence 5785 | AA084560 | dbEST |
| Sequence 5786 | AA086326 | dbEST |
| Sequence 5787 | AA086377 | dbEST |
| Sequence 5788 | AA088544 | dbEST |
| Sequence 5789 | AA088603 | dbEST |
| Sequence 5790 | AA096453 | dbEST |
| Sequence 5791 | AA099875 | dbEST |
| Sequence 5792 | AA099917 | dbEST |
| Sequence 5793 | AA100195 | dbEST |
| Sequence 5794 | AA100852 | dbEST |
| Sequence 5795 | AA101799 | dbEST |
| Sequence 5796 | AA102280 | dbEST |
| Sequence 5797 | AA112936 | dbEST |
| Sequence 5798 | AA113403 | dbEST |
| Sequence 5799 | AA114014 | dbEST |
| Sequence 5800 | AA115512 | dbEST |
| Sequence 5801 | AA115838 | dbEST |
| Sequence 5802 | AA121072 | dbEST |
| Sequence 5803 | AA126009 | dbEST |
| Sequence 5804 | AA126750 | dbEST |
| Sequence 5805 | AA127135 | dbEST |
| Sequence 5806 | AA128104 | dbEST |
| Sequence 5807 | AA128324 | dbEST |
| Sequence 5808 | AA128498 | dbEST |
| Sequence 5809 | AA129245 | dbEST |
| Sequence 5810 | AA129726 | dbEST |
| Sequence 5811 | AA129907 | dbEST |
| Sequence 5812 | AA129962 | dbEST |
| Sequence 5813 | AA130229 | dbEST |
| Sequence 5814 | AA130797 | dbEST |
| Sequence 5815 | AA130946 | dbEST |
| Sequence 5816 | AA131828 | dbEST |
| Sequence 5817 | AA132596 | dbEST |
| Sequence 5818 | AA132844 | dbEST |
| Sequence 5819 | AA133579 | dbEST |
| Sequence 5820 | AA134203 | dbEST |
| Sequence 5821 | AA134234 | dbEST |
| Sequence 5822 | AA134715 | dbEST |
| Sequence 5823 | AA135456 | dbEST |
| Sequence 5824 | AA135663 | dbEST |
| Sequence 5825 | AA136096 | dbEST |
| Sequence 5826 | AA137170 | dbEST |
| Sequence 5827 | AA143286 | dbEST |
| Sequence 5828 | AA143579 | dbEST |
| Sequence 5829 | AA146616 | dbEST |
| Sequence 5830 | AA146701 | dbEST |
| Sequence 5831 | AA147403 | dbEST |
| Sequence 5832 | AA147826 | dbEST |
| Sequence 5833 | AA148019 | dbEST |
| Sequence 5834 | AA148219 | dbEST |
| Sequence 5835 | AA149936 | dbEST |
| Sequence 5836 | AA150320 | dbEST |
| Sequence 5837 | AA150383 | dbEST |
| Sequence 5838 | AA150860 | dbEST |
| Sequence 5839 | AA151350 | dbEST |
| Sequence 5840 | AA151506 | dbEST |
| Sequence 5841 | AA151659 | dbEST |
| Sequence 5842 | AA152009 | dbEST |
| Sequence 5843 | AA152264 | dbEST |
| Sequence 5844 | AA155856 | dbEST |
| Sequence 5845 | AA156215 | dbEST |
| Sequence 5846 | AA157723 | dbEST |
| Sequence 5847 | AA159272 | dbEST |
| Sequence 5848 | AA159748 | dbEST |
| Sequence 5849 | AA160053 | dbEST |
| Sequence 5850 | AA160999 | dbEST |
| Sequence 5851 | AA164607 | dbEST |
| Sequence 5852 | AA164977 | dbEST |
| Sequence 5853 | AA165197 | dbEST |
| Sequence 5854 | AA166675 | dbEST |
| Sequence 5855 | AA167044 | dbEST |
| Sequence 5856 | AA167804 | dbEST |
| Sequence 5857 | AA171510 | dbEST |
| Sequence 5858 | AA171587 | dbEST |
| Sequence 5859 | AA172103 | dbEST |
| Sequence 5860 | AA172156 | dbEST |
| Sequence 5861 | AA173098 | dbEST |
| Sequence 5862 | AA173459 | dbEST |
| Sequence 5863 | AA173952 | dbEST |
| Sequence 5864 | AA176747 | dbEST |
| Sequence 5865 | AA176784 | dbEST |
| Sequence 5866 | AA176979 | dbEST |
| Sequence 5867 | AA179442 | dbEST |
| Sequence 5868 | AA181113 | dbEST |
| Sequence 5869 | AA181392 | dbEST |
| Sequence 5870 | AA182617 | dbEST |
| Sequence 5871 | AA186406 | dbEST |
| Sequence 5872 | AA186432 | dbEST |
| Sequence 5873 | AA186477 | dbEST |
| Sequence 5874 | AA187395 | dbEST |
| Sequence 5875 | AA187762 | dbEST |
| Sequence 5876 | AA188633 | dbEST |
| Sequence 5877 | AA190615 | dbEST |
| Sequence 5878 | AA190873 | dbEST |
| Sequence 5879 | AA190890 | dbEST |
| Sequence 5880 | AA191119 | dbEST |
| Sequence 5881 | AA191211 | dbEST |
| Sequence 5882 | AA192597 | dbEST |
| Sequence 5883 | AA192759 | dbEST |
| Sequence 5884 | AA193297 | dbEST |
| Sequence 5885 | AA193661 | dbEST |
| Sequence 5886 | AA194473 | dbEST |
| Sequence 5887 | AA195015 | dbEST |
| Sequence 5888 | AA195617 | dbEST |
| Sequence 5889 | AA195947 | dbEST |
| Sequence 5890 | AA196357 | dbEST |
| Sequence 5891 | AA203182 | dbEST |
| Sequence 5892 | AA205412 | dbEST |
| Sequence 5893 | AA205546 | dbEST |
| Sequence 5894 | AA206115 | dbEST |
| Sequence 5895 | AA206581 | dbEST |
| Sequence 5896 | AA211799 | dbEST |
| Sequence 5897 | AA214648 | dbEST |
| Sequence 5898 | AA216409 | dbEST |
| Sequence 5899 | AA216669 | dbEST |
| Sequence 5900 | AA218606 | dbEST |
| Sequence 5901 | AA219143 | dbEST |
| Sequence 5902 | AA223148 | dbEST |
| Sequence 5903 | AA224124 | dbEST |
| Sequence 5904 | AA224876 | dbEST |
| Sequence 5905 | AA224985 | dbEST |
| Sequence 5906 | AA225857 | dbEST |
| Sequence 5907 | AA226359 | dbEST |
| Sequence 5908 | AA226898 | dbEST |
| Sequence 5909 | AA227326 | dbEST |
| Sequence 5910 | AA227579 | dbEST |
| Sequence 5911 | AA227837 | dbEST |
| Sequence 5912 | AA228273 | dbEST |
| Sequence 5913 | AA228282 | dbEST |
| Sequence 5914 | AA228836 | dbEST |
| Sequence 5915 | AA228940 | dbEST |
| Sequence 5916 | AA228953 | dbEST |
| Sequence 5917 | AA229057 | dbEST |
| Sequence 5918 | AA229145 | dbEST |
| Sequence 5919 | AA230067 | dbEST |
| Sequence 5920 | AA233733 | dbEST |
| Sequence 5921 | AA235224 | dbEST |
| Sequence 5922 | AA235581 | dbEST |
| Sequence 5923 | AA236371 | dbEST |
| Sequence 5924 | AA236418 | dbEST |
| Sequence 5925 | AA236774 | dbEST |
| Sequence 5926 | AA244003 | dbEST |
| Sequence 5927 | AA244018 | dbEST |
| Sequence 5928 | AA244158 | dbEST |
| Sequence 5929 | AA244184 | dbEST |
| Sequence 5930 | AA248427 | dbEST |
| Sequence 5931 | AA251114 | dbEST |
| Sequence 5932 | AA251678 | dbEST |
| Sequence 5933 | AA256422 | dbEST |
| Sequence 5934 | AA257014 | dbEST |
| Sequence 5935 | AA258089 | dbEST |
| Sequence 5936 | AA278642 | dbEST |
| Sequence 5937 | AA279853 | dbEST |
| Sequence 5938 | AA280091 | dbEST |
| Sequence 5939 | AA281637 | dbEST |
| Sequence 5940 | AA282252 | dbEST |
| Sequence 5941 | AA282382 | dbEST |
| Sequence 5942 | AA283169 | dbEST |
| Sequence 5943 | AA283616 | dbEST |
| Sequence 5944 | AA284615 | dbEST |
| Sequence 5945 | AA284748 | dbEST |
| Sequence 5946 | AA284856 | dbEST |
| Sequence 5947 | AA285290 | dbEST |
| Sequence 5948 | AA287199 | dbEST |
| Sequence 5949 | AA287665 | dbEST |
| Sequence 5950 | AA290783 | dbEST |
| Sequence 5951 | AA291407 | dbEST |
| Sequence 5952 | AA292443 | dbEST |
| Sequence 5953 | AA292786 | dbEST |
| Sequence 5954 | AA293855 | dbEST |
| Sequence 5955 | AA295706 | dbEST |
| Sequence 5956 | AA295762 | dbEST |
| Sequence 5957 | AA296074 | dbEST |
| Sequence 5958 | AA296282 | dbEST |
| Sequence 5959 | AA297097 | dbEST |
| Sequence 5960 | AA297506 | dbEST |
| Sequence 5961 | AA297592 | dbEST |
| Sequence 5962 | AA297750 | dbEST |
| Sequence 5963 | AA298490 | dbEST |
| Sequence 5964 | AA298967 | dbEST |
| Sequence 5965 | AA299668 | dbEST |
| Sequence 5966 | AA299840 | dbEST |
| Sequence 5967 | AA299892 | dbEST |
| Sequence 5968 | AA299961 | dbEST |
| Sequence 5969 | AA300008 | dbEST |
| Sequence 5970 | AA300659 | dbEST |
| Sequence 5971 | AA301205 | dbEST |
| Sequence 5972 | AA301513 | dbEST |
| Sequence 5973 | AA301629 | dbEST |
| Sequence 5974 | AA302313 | dbEST |
| Sequence 5975 | AA302919 | dbEST |
| Sequence 5976 | AA303594 | dbEST |
| Sequence 5977 | AA303960 | dbEST |
| Sequence 5978 | AA304827 | dbEST |
| Sequence 5979 | AA305081 | dbEST |
| Sequence 5980 | AA305121 | dbEST |
| Sequence 5981 | AA305143 | dbEST |
| Sequence 5982 | AA305331 | dbEST |
| Sequence 5983 | AA305333 | dbEST |
| Sequence 5984 | AA305409 | dbEST |
| Sequence 5985 | AA305774 | dbEST |
| Sequence 5986 | AA305819 | dbEST |
| Sequence 5987 | AA305824 | dbEST |
| Sequence 5988 | AA306040 | dbEST |
| Sequence 5989 | AA306182 | dbEST |
| Sequence 5990 | AA306223 | dbEST |
| Sequence 5991 | AA306983 | dbEST |
| Sequence 5992 | AA307033 | dbEST |
| Sequence 5993 | AA307224 | dbEST |
| Sequence 5994 | AA307612 | dbEST |
| Sequence 5995 | AA307669 | dbEST |
| Sequence 5996 | AA307728 | dbEST |
| Sequence 5997 | AA308091 | dbEST |
| Sequence 5998 | AA308207 | dbEST |
| Sequence 5999 | AA308237 | dbEST |
| Sequence 6000 | AA308574 | dbEST |
| Sequence 6001 | AA308812 | dbEST |
| Sequence 6002 | AA309832 | dbEST |
| Sequence 6003 | AA310978 | dbEST |
| Sequence 6004 | AA311044 | dbEST |
| Sequence 6005 | AA311048 | dbEST |
| Sequence 6006 | AA311227 | dbEST |
| Sequence 6007 | AA311677 | dbEST |
| Sequence 6008 | AA311896 | dbEST |
| Sequence 6009 | AA312406 | dbEST |
| Sequence 6010 | AA312864 | dbEST |
| Sequence 6011 | AA313209 | dbEST |
| Sequence 6012 | AA313468 | dbEST |
| Sequence 6013 | AA313653 | dbEST |
| Sequence 6014 | AA314146 | dbEST |
| Sequence 6015 | AA314473 | dbEST |
| Sequence 6016 | AA314673 | dbEST |
| Sequence 6017 | AA314961 | dbEST |
| Sequence 6018 | AA315049 | dbEST |
| Sequence 6019 | AA315188 | dbEST |
| Sequence 6020 | AA315441 | dbEST |
| Sequence 6021 | AA315510 | dbEST |
| Sequence 6022 | AA315950 | dbEST |
| Sequence 6023 | AA315968 | dbEST |
| Sequence 6024 | AA316059 | dbEST |
| Sequence 6025 | AA316217 | dbEST |
| Sequence 6026 | AA316423 | dbEST |
| Sequence 6027 | AA316721 | dbEST |
| Sequence 6028 | AA316723 | dbEST |
| Sequence 6029 | AA317088 | dbEST |
| Sequence 6030 | AA317310 | dbEST |
| Sequence 6031 | AA317477 | dbEST |
| Sequence 6032 | AA317497 | dbEST |
| Sequence 6033 | AA317659 | dbEST |
| Sequence 6034 | AA317855 | dbEST |
| Sequence 6035 | AA317942 | dbEST |
| Sequence 6036 | AA318260 | dbEST |
| Sequence 6037 | AA318272 | dbEST |
| Sequence 6038 | AA320004 | dbEST |
| Sequence 6039 | AA320114 | dbEST |
| Sequence 6040 | AA320723 | dbEST |
| Sequence 6041 | AA321706 | dbEST |
| Sequence 6042 | AA322115 | dbEST |
| Sequence 6043 | AA322352 | dbEST |
| Sequence 6044 | AA323143 | dbEST |
| Sequence 6045 | AA326251 | dbEST |
| Sequence 6046 | AA326895 | dbEST |
| Sequence 6047 | AA327011 | dbEST |
| Sequence 6048 | AA327546 | dbEST |
| Sequence 6049 | AA328770 | dbEST |
| Sequence 6050 | AA330883 | dbEST |
| Sequence 6051 | AA332202 | dbEST |
| Sequence 6052 | AA332737 | dbEST |
| Sequence 6053 | AA334511 | dbEST |
| Sequence 6054 | AA335356 | dbEST |
| Sequence 6055 | AA336197 | dbEST |
| Sequence 6056 | AA337836 | dbEST |
| Sequence 6057 | AA338836 | dbEST |
| Sequence 6058 | AA339065 | dbEST |
| Sequence 6059 | AA340095 | dbEST |
| Sequence 6060 | AA340608 | dbEST |
| Sequence 6061 | AA340634 | dbEST |
| Sequence 6062 | AA340927 | dbEST |
| Sequence 6063 | AA341690 | dbEST |
| Sequence 6064 | AA341987 | dbEST |
| Sequence 6065 | AA343532 | dbEST |
| Sequence 6066 | AA344600 | dbEST |
| Sequence 6067 | AA345762 | dbEST |
| Sequence 6068 | AA345876 | dbEST |
| Sequence 6069 | AA346253 | dbEST |
| Sequence 6070 | AA347236 | dbEST |
| Sequence 6071 | AA347436 | dbEST |
| Sequence 6072 | AA352062 | dbEST |
| Sequence 6073 | AA354376 | dbEST |
| Sequence 6074 | AA355036 | dbEST |
| Sequence 6075 | AA355308 | dbEST |
| Sequence 6076 | AA356436 | dbEST |
| Sequence 6077 | AA357077 | dbEST |
| Sequence 6078 | AA363019 | dbEST |
| Sequence 6079 | AA363481 | dbEST |
| Sequence 6080 | AA364707 | dbEST |
| Sequence 6081 | AA365150 | dbEST |
| Sequence 6082 | AA369660 | dbEST |
| Sequence 6083 | AA369804 | dbEST |
| Sequence 6084 | AA370548 | dbEST |
| Sequence 6085 | AA371017 | dbEST |
| Sequence 6086 | AA371613 | dbEST |
| Sequence 6087 | AA372171 | dbEST |
| Sequence 6088 | AA373314 | dbEST |
| Sequence 6089 | AA374073 | dbEST |
| Sequence 6090 | AA374376 | dbEST |
| Sequence 6091 | AA374561 | dbEST |
| Sequence 6092 | AA375454 | dbEST |
| Sequence 6093 | AA376123 | dbEST |
| Sequence 6094 | AA377218 | dbEST |
| Sequence 6095 | AA384272 | dbEST |
| Sequence 6096 | AA393061 | dbEST |
| Sequence 6097 | AA393485 | dbEST |
| Sequence 6098 | AA393906 | dbEST |
| Sequence 6099 | AA394311 | dbEST |
| Sequence 6100 | AA397452 | dbEST |
| Sequence 6101 | AA397702 | dbEST |
| Sequence 6102 | AA398929 | dbEST |
| Sequence 6103 | AA399024 | dbEST |
| Sequence 6104 | AA399070 | dbEST |
| Sequence 6105 | AA399281 | dbEST |
| Sequence 6106 | AA399632 | dbEST |
| Sequence 6107 | AA400527 | dbEST |
| Sequence 6108 | AA400692 | dbEST |
| Sequence 6109 | AA401501 | dbEST |
| Sequence 6110 | AA401520 | dbEST |
| Sequence 6111 | AA401809 | dbEST |
| Sequence 6112 | AA404376 | dbEST |
| Sequence 6113 | AA404722 | dbEST |
| Sequence 6114 | AA411585 | dbEST |
| Sequence 6115 | AA412302 | dbEST |
| Sequence 6116 | AA412500 | dbEST |
| Sequence 6117 | AA417643 | dbEST |
| Sequence 6118 | AA417842 | dbEST |
| Sequence 6119 | AA420435 | dbEST |
| Sequence 6120 | AA420462 | dbEST |
| Sequence 6121 | AA420758 | dbEST |
| Sequence 6122 | AA420800 | dbEST |
| Sequence 6123 | AA420825 | dbEST |
| Sequence 6124 | AA420845 | dbEST |
| Sequence 6125 | AA422173 | dbEST |
| Sequence 6126 | AA424243 | dbEST |
| Sequence 6127 | AA425212 | dbEST |
| Sequence 6128 | AA425305 | dbEST |
| Sequence 6129 | AA425460 | dbEST |
| Sequence 6130 | AA425492 | dbEST |
| Sequence 6131 | AA425509 | dbEST |
| Sequence 6132 | AA425583 | dbEST |
| Sequence 6133 | AA426351 | dbEST |
| Sequence 6134 | AA428216 | dbEST |
| Sequence 6135 | AA431579 | dbEST |
| Sequence 6136 | AA433869 | dbEST |
| Sequence 6137 | AA434286 | dbEST |
| Sequence 6138 | AA435894 | dbEST |
| Sequence 6139 | AA436394 | dbEST |
| Sequence 6140 | AA436652 | dbEST |
| Sequence 6141 | AA437304 | dbEST |
| Sequence 6142 | AA442017 | dbEST |
| Sequence 6143 | AA442145 | dbEST |
| Sequence 6144 | AA442287 | dbEST |
| Sequence 6145 | AA442517 | dbEST |
| Sequence 6146 | AA443757 | dbEST |
| Sequence 6147 | AA443834 | dbEST |
| Sequence 6148 | AA444072 | dbEST |
| Sequence 6149 | AA444386 | dbEST |
| Sequence 6150 | AA446103 | dbEST |
| Sequence 6151 | AA447349 | dbEST |
| Sequence 6152 | AA447563 | dbEST |
| Sequence 6153 | AA448526 | dbEST |
| Sequence 6154 | AA449228 | dbEST |
| Sequence 6155 | AA450120 | dbEST |
| Sequence 6156 | AA452177 | dbEST |
| Sequence 6157 | AA453134 | dbEST |
| Sequence 6158 | AA453310 | dbEST |
| Sequence 6159 | AA453333 | dbEST |
| Sequence 6160 | AA453478 | dbEST |
| Sequence 6161 | AA453719 | dbEST |
| Sequence 6162 | AA454962 | dbEST |
| Sequence 6163 | AA456246 | dbEST |
| Sequence 6164 | AA459208 | dbEST |
| Sequence 6165 | AA459871 | dbEST |
| Sequence 6166 | AA460440 | dbEST |
| Sequence 6167 | AA463469 | dbEST |
| Sequence 6168 | AA464160 | dbEST |
| Sequence 6169 | AA464338 | dbEST |
| Sequence 6170 | AA465494 | dbEST |
| Sequence 6171 | AA467748 | dbEST |
| Sequence 6172 | AA467761 | dbEST |
| Sequence 6173 | AA468151 | dbEST |
| Sequence 6174 | AA468167 | dbEST |
| Sequence 6175 | AA468504 | dbEST |
| Sequence 6176 | AA468760 | dbEST |
| Sequence 6177 | AA469129 | dbEST |
| Sequence 6178 | AA469151 | dbEST |
| Sequence 6179 | AA469348 | dbEST |
| Sequence 6180 | AA469926 | dbEST |
| Sequence 6181 | AA469979 | dbEST |
| Sequence 6182 | AA470575 | dbEST |
| Sequence 6183 | AA470602 | dbEST |
| Sequence 6184 | AA471056 | dbEST |
| Sequence 6185 | AA477075 | dbEST |
| Sequence 6186 | AA477397 | dbEST |
| Sequence 6187 | AA477803 | dbEST |
| Sequence 6188 | AA478415 | dbEST |
| Sequence 6189 | AA479151 | dbEST |
| Sequence 6190 | AA479167 | dbEST |
| Sequence 6191 | AA480124 | dbEST |
| Sequence 6192 | AA480282 | dbEST |
| Sequence 6193 | AA480529 | dbEST |
| Sequence 6194 | AA481738 | dbEST |
| Sequence 6195 | AA484711 | dbEST |
| Sequence 6196 | AA484821 | dbEST |
| Sequence 6197 | AA485467 | dbEST |
| Sequence 6198 | AA485853 | dbEST |
| Sequence 6199 | AA486619 | dbEST |
| Sequence 6200 | AA486628 | dbEST |
| Sequence 6201 | AA486837 | dbEST |
| Sequence 6202 | AA487192 | dbEST |
| Sequence 6203 | AA488014 | dbEST |
| Sequence 6204 | AA489323 | dbEST |
| Sequence 6205 | AA490047 | dbEST |
| Sequence 6206 | AA491272 | dbEST |
| Sequence 6207 | AA491302 | dbEST |
| Sequence 6208 | AA492026 | dbEST |
| Sequence 6209 | AA492038 | dbEST |
| Sequence 6210 | AA492042 | dbEST |
| Sequence 6211 | AA492219 | dbEST |
| Sequence 6212 | AA492280 | dbEST |
| Sequence 6213 | AA492390 | dbEST |
| Sequence 6214 | AA496932 | dbEST |
| Sequence 6215 | AA501968 | dbEST |
| Sequence 6216 | AA502897 | dbEST |
| Sequence 6217 | AA502955 | dbEST |
| Sequence 6218 | AA502979 | dbEST |
| Sequence 6219 | AA503718 | dbEST |
| Sequence 6220 | AA503930 | dbEST |
| Sequence 6221 | AA503943 | dbEST |
| Sequence 6222 | AA504173 | dbEST |
| Sequence 6223 | AA504246 | dbEST |
| Sequence 6224 | AA506607 | dbEST |
| Sequence 6225 | AA506923 | dbEST |
| Sequence 6226 | AA506967 | dbEST |
| Sequence 6227 | AA507084 | dbEST |
| Sequence 6228 | AA507511 | dbEST |
| Sequence 6229 | AA508552 | dbEST |
| Sequence 6230 | AA508579 | dbEST |
| Sequence 6231 | AA508898 | dbEST |
| Sequence 6232 | AA513056 | dbEST |
| Sequence 6233 | AA513597 | dbEST |
| Sequence 6234 | AA514042 | dbEST |
| Sequence 6235 | AA515576 | dbEST |
| Sequence 6236 | AA515857 | dbEST |
| Sequence 6237 | AA516531 | dbEST |
| Sequence 6238 | AA521140 | dbEST |
| Sequence 6239 | AA523252 | dbEST |
| Sequence 6240 | AA523671 | dbEST |
| Sequence 6241 | AA523902 | dbEST |
| Sequence 6242 | AA524651 | dbEST |
| Sequence 6243 | AA525032 | dbEST |
| Sequence 6244 | AA525419 | dbEST |
| Sequence 6245 | AA525497 | dbEST |
| Sequence 6246 | AA525958 | dbEST |
| Sequence 6247 | AA526244 | dbEST |
| Sequence 6248 | AA526283 | dbEST |
| Sequence 6249 | AA526834 | dbEST |
| Sequence 6250 | AA527667 | dbEST |
| Sequence 6251 | AA527805 | dbEST |
| Sequence 6252 | AA528204 | dbEST |
| Sequence 6253 | AA531198 | dbEST |
| Sequence 6254 | AA531509 | dbEST |
| Sequence 6255 | AA533115 | dbEST |
| Sequence 6256 | AA533386 | dbEST |
| Sequence 6257 | AA533494 | dbEST |
| Sequence 6258 | AA533709 | dbEST |
| Sequence 6259 | AA533785 | dbEST |
| Sequence 6260 | AA534246 | dbEST |
| Sequence 6261 | AA534778 | dbEST |
| Sequence 6262 | AA535218 | dbEST |
| Sequence 6263 | AA535384 | dbEST |
| Sequence 6264 | AA535695 | dbEST |
| Sequence 6265 | AA541677 | dbEST |
| Sequence 6266 | AA548308 | dbEST |
| Sequence 6267 | AA550927 | dbEST |
| Sequence 6268 | AA551894 | dbEST |
| Sequence 6269 | AA552321 | dbEST |
| Sequence 6270 | AA552842 | dbEST |
| Sequence 6271 | AA558611 | dbEST |
| Sequence 6272 | AA558636 | dbEST |
| Sequence 6273 | AA558976 | dbEST |
| Sequence 6274 | AA564277 | dbEST |
| Sequence 6275 | AA564484 | dbEST |
| Sequence 6276 | AA565996 | dbEST |
| Sequence 6277 | AA569344 | dbEST |
| Sequence 6278 | AA569434 | dbEST |
| Sequence 6279 | AA569482 | dbEST |
| Sequence 6280 | AA573368 | dbEST |
| Sequence 6281 | AA573646 | dbEST |
| Sequence 6282 | AA573742 | dbEST |
| Sequence 6283 | AA574288 | dbEST |
| Sequence 6284 | AA574397 | dbEST |
| Sequence 6285 | AA574436 | dbEST |
| Sequence 6286 | AA576243 | dbEST |
| Sequence 6287 | AA576407 | dbEST |
| Sequence 6288 | AA578009 | dbEST |
| Sequence 6289 | AA578577 | dbEST |
| Sequence 6290 | AA578646 | dbEST |
| Sequence 6291 | AA578773 | dbEST |
| Sequence 6292 | AA579039 | dbEST |
| Sequence 6293 | AA579904 | dbEST |
| Sequence 6294 | AA580097 | dbEST |
| Sequence 6295 | AA580829 | dbEST |
| Sequence 6296 | AA581716 | dbEST |
| Sequence 6297 | AA583350 | dbEST |
| Sequence 6298 | AA583353 | dbEST |
| Sequence 6299 | AA587020 | dbEST |
| Sequence 6300 | AA587243 | dbEST |
| Sequence 6301 | AA587459 | dbEST |
| Sequence 6302 | AA588685 | dbEST |
| Sequence 6303 | AA588754 | dbEST |
| Sequence 6304 | AA588882 | dbEST |
| Sequence 6305 | AA593159 | dbEST |
| Sequence 6306 | AA593668 | dbEST |
| Sequence 6307 | AA595100 | dbEST |
| Sequence 6308 | AA599416 | dbEST |
| Sequence 6309 | AA600257 | dbEST |
| Sequence 6310 | AA603265 | dbEST |
| Sequence 6311 | AA603834 | dbEST |
| Sequence 6312 | AA613908 | dbEST |
| Sequence 6313 | AA618486 | dbEST |
| Sequence 6314 | AA620750 | dbEST |
| Sequence 6315 | AA621031 | dbEST |
| Sequence 6316 | AA621073 | dbEST |
| Sequence 6317 | AA621820 | dbEST |
| Sequence 6318 | AA625415 | dbEST |
| Sequence 6319 | AA625485 | dbEST |
| Sequence 6320 | AA625833 | dbEST |
| Sequence 6321 | AA626161 | dbEST |
| Sequence 6322 | AA626274 | dbEST |
| Sequence 6323 | AA626439 | dbEST |
| Sequence 6324 | AA628530 | dbEST |
| Sequence 6325 | AA629325 | dbEST |
| Sequence 6326 | AA629581 | dbEST |
| Sequence 6327 | AA630298 | dbEST |
| Sequence 6328 | AA631446 | dbEST |
| Sequence 6329 | AA631868 | dbEST |
| Sequence 6330 | AA632427 | dbEST |
| Sequence 6331 | AA633399 | dbEST |
| Sequence 6332 | AA633846 | dbEST |
| Sequence 6333 | AA635288 | dbEST |
| Sequence 6334 | AA640133 | dbEST |
| Sequence 6335 | AA640574 | dbEST |
| Sequence 6336 | AA640889 | dbEST |
| Sequence 6337 | AA641585 | dbEST |
| Sequence 6338 | AA641623 | dbEST |
| Sequence 6339 | AA642495 | dbEST |
| Sequence 6340 | AA648194 | dbEST |
| Sequence 6341 | AA652478 | dbEST |
| Sequence 6342 | AA654527 | dbEST |
| Sequence 6343 | AA657830 | dbEST |
| Sequence 6344 | AA659142 | dbEST |
| Sequence 6345 | AA659391 | dbEST |
| Sequence 6346 | AA662111 | dbEST |
| Sequence 6347 | AA662369 | dbEST |
| Sequence 6348 | AA662822 | dbEST |
| Sequence 6349 | AA663453 | dbEST |
| Sequence 6350 | AA664480 | dbEST |
| Sequence 6351 | AA669438 | dbEST |
| Sequence 6352 | AA670205 | dbEST |
| Sequence 6353 | AA677550 | dbEST |
| Sequence 6354 | AA687179 | dbEST |
| Sequence 6355 | AA687216 | dbEST |
| Sequence 6356 | AA687370 | dbEST |
| Sequence 6357 | AA687538 | dbEST |
| Sequence 6358 | AA687674 | dbEST |
| Sequence 6359 | AA687718 | dbEST |
| Sequence 6360 | AA693742 | dbEST |
| Sequence 6361 | AA694055 | dbEST |
| Sequence 6362 | AA694120 | dbEST |
| Sequence 6363 | AA700509 | dbEST |
| Sequence 6364 | AA701607 | dbEST |
| Sequence 6365 | AA702084 | dbEST |
| Sequence 6366 | AA702131 | dbEST |
| Sequence 6367 | AA702891 | dbEST |
| Sequence 6368 | AA703838 | dbEST |
| Sequence 6369 | AA703925 | dbEST |
| Sequence 6370 | AA704323 | dbEST |
| Sequence 6371 | AA705957 | dbEST |
| Sequence 6372 | AA705986 | dbEST |
| Sequence 6373 | AA706314 | dbEST |
| Sequence 6374 | AA707377 | dbEST |
| Sequence 6375 | AA707750 | dbEST |
| Sequence 6376 | AA708457 | dbEST |
| Sequence 6377 | AA709388 | dbEST |
| Sequence 6378 | AA714481 | dbEST |
| Sequence 6379 | AA716097 | dbEST |
| Sequence 6380 | AA719530 | dbEST |
| Sequence 6381 | AA725298 | dbEST |
| Sequence 6382 | AA725836 | dbEST |
| Sequence 6383 | AA732352 | dbEST |
| Sequence 6384 | AA745678 | dbEST |
| Sequence 6385 | AA747515 | dbEST |
| Sequence 6386 | AA748033 | dbEST |
| Sequence 6387 | AA758592 | dbEST |
| Sequence 6388 | AA761739 | dbEST |
| Sequence 6389 | AA768762 | dbEST |
| Sequence 6390 | AA774265 | dbEST |
| Sequence 6391 | AA775625 | dbEST |
| Sequence 6392 | AA776709 | dbEST |
| Sequence 6393 | AA779427 | dbEST |
| Sequence 6394 | AA805361 | dbEST |
| Sequence 6395 | AA806555 | dbEST |
| Sequence 6396 | AA813373 | dbEST |
| Sequence 6397 | AA814822 | dbEST |
| Sequence 6398 | AA824416 | dbEST |
| Sequence 6399 | AA825612 | dbEST |
| Sequence 6400 | AA827079 | dbEST |
| Sequence 6401 | AA827968 | dbEST |
| Sequence 6402 | AA828243 | dbEST |
| Sequence 6403 | AA830856 | dbEST |
| Sequence 6404 | AA831148 | dbEST |
| Sequence 6405 | AA835638 | dbEST |
| Sequence 6406 | AA838377 | dbEST |
| Sequence 6407 | AA838431 | dbEST |
| Sequence 6408 | AA838482 | dbEST |
| Sequence 6409 | AA843133 | dbEST |
| Sequence 6410 | AA845390 | dbEST |
| Sequence 6411 | AA846335 | dbEST |
| Sequence 6412 | AA852998 | dbEST |
| Sequence 6413 | AA857872 | dbEST |
| Sequence 6414 | AA861986 | dbEST |
| Sequence 6415 | AA862309 | dbEST |
| Sequence 6416 | AA864840 | dbEST |
| Sequence 6417 | AA872710 | dbEST |
| Sequence 6418 | AA873534 | dbEST |
| Sequence 6419 | AA877864 | dbEST |
| Sequence 6420 | AA883508 | dbEST |
| Sequence 6421 | AA885780 | dbEST |
| Sequence 6422 | AA885835 | dbEST |
| Sequence 6423 | AA887235 | dbEST |
| Sequence 6424 | AA890032 | dbEST |
| Sequence 6425 | AA894781 | dbEST |
| Sequence 6426 | AA894929 | dbEST |
| Sequence 6427 | AA902127 | dbEST |
| Sequence 6428 | AA902256 | dbEST |
| Sequence 6429 | AA903809 | dbEST |
| Sequence 6430 | AA904647 | dbEST |
| Sequence 6431 | AA905065 | dbEST |
| Sequence 6432 | AA908930 | dbEST |
| Sequence 6433 | AA909977 | dbEST |
| Sequence 6434 | AA911903 | dbEST |
| Sequence 6435 | AA912713 | dbEST |
| Sequence 6436 | AA917937 | dbEST |
| Sequence 6437 | AA923547 | dbEST |
| Sequence 6438 | AA926656 | dbEST |
| Sequence 6439 | AA928427 | dbEST |
| Sequence 6440 | AA932331 | dbEST |
| Sequence 6441 | AA934851 | dbEST |
| Sequence 6442 | AA935485 | dbEST |
| Sequence 6443 | AA935560 | dbEST |
| Sequence 6444 | AA936089 | dbEST |
| Sequence 6445 | AA937513 | dbEST |
| Sequence 6446 | AA937773 | dbEST |
| Sequence 6447 | AA938181 | dbEST |
| Sequence 6448 | AA938717 | dbEST |
| Sequence 6449 | AA939023 | dbEST |
| Sequence 6450 | AA948276 | dbEST |
| Sequence 6451 | AA970266 | dbEST |
| Sequence 6452 | AA972530 | dbEST |
| Sequence 6453 | AA972602 | dbEST |
| Sequence 6454 | AA974137 | dbEST |
| Sequence 6455 | AA975275 | dbEST |
| Sequence 6456 | AA977226 | dbEST |
| Sequence 6457 | AA977833 | dbEST |
| Sequence 6458 | AA978353 | dbEST |
| Sequence 6459 | AA983560 | dbEST |
| Sequence 6460 | AA985190 | dbEST |
| Sequence 6461 | AA994534 | dbEST |
| Sequence 6462 | AA995483 | dbEST |
| Sequence 6463 | AF017688 | dbEST |
| Sequence 6464 | AF034176 | dbEST |
| Sequence 6465 | AI015053 | dbEST |
| Sequence 6466 | AI016700 | dbEST |
| Sequence 6467 | AI018625 | dbEST |
| Sequence 6468 | AI022134 | dbEST |
| Sequence 6469 | AI022855 | dbEST |
| Sequence 6470 | AI023931 | dbEST |
| Sequence 6471 | AI027887 | dbEST |
| Sequence 6472 | AI028122 | dbEST |
| Sequence 6473 | AI028708 | dbEST |
| Sequence 6474 | AI032401 | dbEST |
| Sequence 6475 | AI034431 | dbEST |
| Sequence 6476 | AI041847 | dbEST |
| Sequence 6477 | AI042059 | dbEST |
| Sequence 6478 | AI052112 | dbEST |
| Sequence 6479 | AI055870 | dbEST |
| Sequence 6480 | AI056489 | dbEST |
| Sequence 6481 | AI057142 | dbEST |
| Sequence 6482 | AI074397 | dbEST |
| Sequence 6483 | AI074613 | dbEST |
| Sequence 6484 | AI078381 | dbEST |
| Sequence 6485 | AI080170 | dbEST |
| Sequence 6486 | AI080267 | dbEST |
| Sequence 6487 | AI080485 | dbEST |
| Sequence 6488 | AI081291 | dbEST |
| Sequence 6489 | AI084101 | dbEST |
| Sequence 6490 | AI084916 | dbEST |
| Sequence 6491 | AI085393 | dbEST |
| Sequence 6492 | AI087412 | dbEST |
| Sequence 6493 | AI089394 | dbEST |
| Sequence 6494 | AI089526 | dbEST |
| Sequence 6495 | AI089948 | dbEST |
| Sequence 6496 | AI090786 | dbEST |
| Sequence 6497 | AI091736 | dbEST |
| Sequence 6498 | AI095875 | dbEST |
| Sequence 6499 | AI096493 | dbEST |
| Sequence 6500 | AI123916 | dbEST |
| Sequence 6501 | AI124532 | dbEST |
| Sequence 6502 | AI125642 | dbEST |
| Sequence 6503 | AI127167 | dbEST |
| Sequence 6504 | AI127176 | dbEST |
| Sequence 6505 | AI127424 | dbEST |
| Sequence 6506 | AI127455 | dbEST |
| Sequence 6507 | AI127556 | dbEST |
| Sequence 6508 | AI128918 | dbEST |
| Sequence 6509 | AI129342 | dbEST |
| Sequence 6510 | AI129403 | dbEST |
| Sequence 6511 | AI133096 | dbEST |
| Sequence 6512 | AI133152 | dbEST |
| Sequence 6513 | AI133330 | dbEST |
| Sequence 6514 | AI133381 | dbEST |
| Sequence 6515 | AI133402 | dbEST |
| Sequence 6516 | AI139036 | dbEST |
| Sequence 6517 | AI139157 | dbEST |
| Sequence 6518 | AI140251 | dbEST |
| Sequence 6519 | AI140421 | dbEST |
| Sequence 6520 | AI141483 | dbEST |
| Sequence 6521 | AI142083 | dbEST |
| Sequence 6522 | AI148437 | dbEST |
| Sequence 6523 | AI149743 | dbEST |
| Sequence 6524 | AI151482 | dbEST |
| Sequence 6525 | AI160324 | dbEST |
| Sequence 6526 | AI161333 | dbEST |
| Sequence 6527 | AI174828 | dbEST |
| Sequence 6528 | AI186545 | dbEST |
| Sequence 6529 | AI188641 | dbEST |
| Sequence 6530 | AI190480 | dbEST |
| Sequence 6531 | AI192932 | dbEST |
| Sequence 6532 | AI197946 | dbEST |
| Sequence 6533 | AI198625 | dbEST |
| Sequence 6534 | AI199544 | dbEST |
| Sequence 6535 | AI200798 | dbEST |
| Sequence 6536 | AI202026 | dbEST |
| Sequence 6537 | AI203141 | dbEST |
| Sequence 6538 | AI204308 | dbEST |
| Sequence 6539 | AI205228 | dbEST |
| Sequence 6540 | AI206063 | dbEST |
| Sequence 6541 | AI207781 | dbEST |
| Sequence 6542 | AI216969 | dbEST |
| Sequence 6543 | AI217009 | dbEST |
| Sequence 6544 | AI217172 | dbEST |
| Sequence 6545 | AI217964 | dbEST |
| Sequence 6546 | AI219278 | dbEST |
| Sequence 6547 | AI220051 | dbEST |
| Sequence 6548 | AI239506 | dbEST |
| Sequence 6549 | AI241117 | dbEST |
| Sequence 6550 | AI241659 | dbEST |
| Sequence 6551 | AI241744 | dbEST |
| Sequence 6552 | AI246383 | dbEST |
| Sequence 6553 | AI249764 | dbEST |
| Sequence 6554 | AI250055 | dbEST |
| Sequence 6555 | AI252084 | dbEST |
| Sequence 6556 | AI252283 | dbEST |
| Sequence 6557 | AI253099 | dbEST |
| Sequence 6558 | AI253330 | dbEST |
| Sequence 6559 | AI253335 | dbEST |
| Sequence 6560 | AI253363 | dbEST |
| Sequence 6561 | AI261763 | dbEST |
| Sequence 6562 | AI267185 | dbEST |
| Sequence 6563 | AI267282 | dbEST |
| Sequence 6564 | AI267502 | dbEST |
| Sequence 6565 | AI269158 | dbEST |
| Sequence 6566 | AI273636 | dbEST |
| Sequence 6567 | AI276399 | dbEST |
| Sequence 6568 | AI276576 | dbEST |
| Sequence 6569 | AI276662 | dbEST |
| Sequence 6570 | AI278014 | dbEST |
| Sequence 6571 | AI281196 | dbEST |
| Sequence 6572 | AI281926 | dbEST |
| Sequence 6573 | AI282318 | dbEST |
| Sequence 6574 | AI298059 | dbEST |
| Sequence 6575 | AI300532 | dbEST |
| Sequence 6576 | AI300553 | dbEST |
| Sequence 6577 | AI302799 | dbEST |
| Sequence 6578 | AI304935 | dbEST |
| Sequence 6579 | AI310138 | dbEST |
| Sequence 6580 | AI312325 | dbEST |
| Sequence 6581 | AI318091 | dbEST |
| Sequence 6582 | AI335028 | dbEST |
| Sequence 6583 | AI335178 | dbEST |
| Sequence 6584 | AI336501 | dbEST |
| Sequence 6585 | AI336800 | dbEST |
| Sequence 6586 | AI336949 | dbEST |
| Sequence 6587 | AI338603 | dbEST |
| Sequence 6588 | AI339354 | dbEST |
| Sequence 6589 | AI340981 | dbEST |
| Sequence 6590 | AI341972 | dbEST |
| Sequence 6591 | AI342546 | dbEST |
| Sequence 6592 | AI342937 | dbEST |
| Sequence 6593 | AI343692 | dbEST |
| Sequence 6594 | AI346657 | dbEST |
| Sequence 6595 | AI347473 | dbEST |
| Sequence 6596 | AI349099 | dbEST |
| Sequence 6597 | AI349937 | dbEST |
| Sequence 6598 | AI357472 | dbEST |
| Sequence 6599 | AI360165 | dbEST |
| Sequence 6600 | AI366549 | dbEST |
| Sequence 6601 | AI368743 | dbEST |
| Sequence 6602 | AI369849 | dbEST |
| Sequence 6603 | AI377012 | dbEST |
| Sequence 6604 | AI378613 | dbEST |
| Sequence 6605 | AI380537 | dbEST |
| Sequence 6606 | AI380842 | dbEST |
| Sequence 6607 | AI401771 | dbEST |
| Sequence 6608 | AI417016 | dbEST |
| Sequence 6609 | AI421249 | dbEST |
| Sequence 6610 | AI431350 | dbEST |
| Sequence 6611 | AI433818 | dbEST |
| Sequence 6612 | AI434250 | dbEST |
| Sequence 6613 | AI435349 | dbEST |
| Sequence 6614 | AI439987 | dbEST |
| Sequence 6615 | AI446249 | dbEST |
| Sequence 6616 | AI452707 | dbEST |
| Sequence 6617 | AI453402 | dbEST |
| Sequence 6618 | AI474125 | dbEST |
| Sequence 6619 | AI475810 | dbEST |
| Sequence 6620 | AI479948 | dbEST |
| Sequence 6621 | AI491775 | dbEST |
| Sequence 6622 | AI499331 | dbEST |
| Sequence 6623 | AI525654 | dbEST |
| Sequence 6624 | AI525843 | dbEST |
| Sequence 6625 | AI536833 | dbEST |
| Sequence 6626 | AI547041 | dbEST |
| Sequence 6627 | AI547068 | dbEST |
| Sequence 6628 | AI547090 | dbEST |
| Sequence 6629 | AI547125 | dbEST |
| Sequence 6630 | AI547285 | dbEST |
| Sequence 6631 | AI557225 | dbEST |
| Sequence 6632 | AI557231 | dbEST |
| Sequence 6633 | AI557246 | dbEST |
| Sequence 6634 | AI557269 | dbEST |
| Sequence 6635 | AI557458 | dbEST |
| Sequence 6636 | AI557495 | dbEST |
| Sequence 6637 | AI563921 | dbEST |
| Sequence 6638 | AI566084 | dbEST |
| Sequence 6639 | AI570349 | dbEST |
| Sequence 6640 | AI612873 | dbEST |
| Sequence 6641 | AI627356 | dbEST |
| Sequence 6642 | AI630118 | dbEST |
| Sequence 6643 | AI630685 | dbEST |
| Sequence 6644 | AI630985 | dbEST |
| Sequence 6645 | AI631745 | dbEST |
| Sequence 6646 | AI632244 | dbEST |
| Sequence 6647 | AI632534 | dbEST |
| Sequence 6648 | AI636701 | dbEST |
| Sequence 6649 | AI653367 | dbEST |
| Sequence 6650 | AI654122 | dbEST |
| Sequence 6651 | AI659898 | dbEST |
| Sequence 6652 | AI660243 | dbEST |
| Sequence 6653 | AI669052 | dbEST |
| Sequence 6654 | AI674586 | dbEST |
| Sequence 6655 | AI680416 | dbEST |
| Sequence 6656 | AI680506 | dbEST |
| Sequence 6657 | AI684170 | dbEST |
| Sequence 6658 | AI691072 | dbEST |
| Sequence 6659 | AI696513 | dbEST |
| Sequence 6660 | AI700228 | dbEST |
| Sequence 6661 | AI719425 | dbEST |
| Sequence 6662 | AI719701 | dbEST |
| Sequence 6663 | AI720354 | dbEST |
| Sequence 6664 | AI732097 | dbEST |
| Sequence 6665 | AI734266 | dbEST |
| Sequence 6666 | AI734854 | dbEST |
| Sequence 6667 | AI735683 | dbEST |
| Sequence 6668 | AI740869 | dbEST |
| Sequence 6669 | AI741192 | dbEST |
| Sequence 6670 | AI749547 | dbEST |
| Sequence 6671 | AI751930 | dbEST |
| Sequence 6672 | AI752546 | dbEST |
| Sequence 6673 | AI752913 | dbEST |
| Sequence 6674 | AI753229 | dbEST |
| Sequence 6675 | AI753662 | dbEST |
| Sequence 6676 | AI753951 | dbEST |
| Sequence 6677 | AI754071 | dbEST |
| Sequence 6678 | AI755085 | dbEST |
| Sequence 6679 | AI796120 | dbEST |
| Sequence 6680 | AI810698 | dbEST |
| Sequence 6681 | AI813937 | dbEST |
| Sequence 6682 | AI815677 | dbEST |
| Sequence 6683 | AI815703 | dbEST |
| Sequence 6684 | AI815972 | dbEST |
| Sequence 6685 | AI815987 | dbEST |
| Sequence 6686 | AI816235 | dbEST |
| Sequence 6687 | AI816313 | dbEST |
| Sequence 6688 | AI816938 | dbEST |
| Sequence 6689 | AI818255 | dbEST |
| Sequence 6690 | AI825504 | dbEST |
| Sequence 6691 | AI825799 | dbEST |
| Sequence 6692 | AI859156 | dbEST |
| Sequence 6693 | AI878846 | dbEST |
| Sequence 6694 | AI879036 | dbEST |
| Sequence 6695 | AI884963 | dbEST |
| Sequence 6696 | AI889347 | dbEST |
| Sequence 6697 | AI914133 | dbEST |
| Sequence 6698 | AI917110 | dbEST |
| Sequence 6699 | AI929339 | dbEST |
| Sequence 6700 | AI934595 | dbEST |
| Sequence 6701 | AI951049 | dbEST |
| Sequence 6702 | AI952286 | dbEST |
| Sequence 6703 | AI961413 | dbEST |
| Sequence 6704 | AI968069 | dbEST |
| Sequence 6705 | AI969804 | dbEST |
| Sequence 6706 | AI978832 | dbEST |
| Sequence 6707 | AL035756 | dbEST |
| Sequence 6708 | AL036801 | dbEST |
| Sequence 6709 | AL036890 | dbEST |
| Sequence 6710 | AL037798 | dbEST |
| Sequence 6711 | AL038242 | dbEST |
| Sequence 6712 | AL038395 | dbEST |
| Sequence 6713 | AL039175 | dbEST |
| Sequence 6714 | AL039507 | dbEST |
| Sequence 6715 | AL040632 | dbEST |
| Sequence 6716 | AL040676 | dbEST |
| Sequence 6717 | AL044040 | dbEST |
| Sequence 6718 | AL045977 | dbEST |
| Sequence 6719 | AL046005 | dbEST |
| Sequence 6720 | AL046079 | dbEST |
| Sequence 6721 | AL048748 | dbEST |
| Sequence 6722 | AL049145 | dbEST |
| Sequence 6723 | AL118746 | dbEST |
| Sequence 6724 | AL119449 | dbEST |
| Sequence 6725 | AL119462 | dbEST |
| Sequence 6726 | AL120558 | dbEST |
| Sequence 6727 | AL120741 | dbEST |
| Sequence 6728 | AL120789 | dbEST |
| Sequence 6729 | AL121220 | dbEST |
| Sequence 6730 | AL121488 | dbEST |
| Sequence 6731 | AW003821 | dbEST |
| Sequence 6732 | AW003952 | dbEST |
| Sequence 6733 | AW005947 | dbEST |
| Sequence 6734 | AW015229 | dbEST |
| Sequence 6735 | AW015828 | dbEST |
| Sequence 6736 | AW021832 | dbEST |
| Sequence 6737 | AW026131 | dbEST |
| Sequence 6738 | AW026502 | dbEST |
| Sequence 6739 | AW055209 | dbEST |
| Sequence 6740 | AW068372 | dbEST |
| Sequence 6741 | AW082236 | dbEST |
| Sequence 6742 | AW087623 | dbEST |
| Sequence 6743 | AW090304 | dbEST |
| Sequence 6744 | AW102895 | dbEST |
| Sequence 6745 | AW130969 | dbEST |
| Sequence 6746 | AW135062 | dbEST |
| Sequence 6747 | AW135187 | dbEST |
| Sequence 6748 | AW157303 | dbEST |
| Sequence 6749 | AW160399 | dbEST |
| Sequence 6750 | AW160696 | dbEST |
| Sequence 6751 | AW161209 | dbEST |
| Sequence 6752 | AW161365 | dbEST |
| Sequence 6753 | AW161467 | dbEST |
| Sequence 6754 | AW162502 | dbEST |
| Sequence 6755 | AW176042 | dbEST |
| Sequence 6756 | AW178342 | dbEST |
| Sequence 6757 | AW179006 | dbEST |
| Sequence 6758 | C03130 | dbEST |
| Sequence 6759 | C18148 | dbEST |
| Sequence 6760 | C18654 | dbEST |
| Sequence 6761 | D12135 | dbEST |
| Sequence 6762 | D30921 | dbEST |
| Sequence 6763 | D58694 | dbEST |
| Sequence 6764 | D61285 | dbEST |
| Sequence 6765 | D82149 | dbEST |
| Sequence 6766 | D82176 | dbEST |
| Sequence 6767 | F01265 | dbEST |
| Sequence 6768 | F01327 | dbEST |
| Sequence 6769 | F05508 | dbEST |
| Sequence 6770 | H02725 | dbEST |
| Sequence 6771 | H04072 | dbEST |
| Sequence 6772 | H10429 | dbEST |
| Sequence 6773 | H10482 | dbEST |
| Sequence 6774 | H12109 | dbEST |
| Sequence 6775 | H12519 | dbEST |
| Sequence 6776 | H13339 | dbEST |
| Sequence 6777 | H17773 | dbEST |
| Sequence 6778 | H19393 | dbEST |
| Sequence 6779 | H38564 | dbEST |
| Sequence 6780 | H47325 | dbEST |
| Sequence 6781 | H48697 | dbEST |
| Sequence 6782 | H51749 | dbEST |
| Sequence 6783 | H54240 | dbEST |
| Sequence 6784 | H59731 | dbEST |
| Sequence 6785 | H66398 | dbEST |
| Sequence 6786 | H82596 | dbEST |
| Sequence 6787 | H84670 | dbEST |
| Sequence 6788 | H89582 | dbEST |
| Sequence 6789 | H93264 | dbEST |
| Sequence 6790 | H99333 | dbEST |
| Sequence 6791 | N23771 | dbEST |
| Sequence 6792 | N39674 | dbEST |
| Sequence 6793 | N39836 | dbEST |
| Sequence 6794 | N40375 | dbEST |
| Sequence 6795 | N42929 | dbEST |
| Sequence 6796 | N44743 | dbEST |
| Sequence 6797 | N47292 | dbEST |
| Sequence 6798 | N49346 | dbEST |
| Sequence 6799 | N56237 | dbEST |
| Sequence 6800 | N62799 | dbEST |
| Sequence 6801 | N63387 | dbEST |
| Sequence 6802 | N75468 | dbEST |
| Sequence 6803 | N79712 | dbEST |
| Sequence 6804 | N94351 | dbEST |
| Sequence 6805 | N98328 | dbEST |
| Sequence 6806 | N99088 | dbEST |
| Sequence 6807 | R12356 | dbEST |
| Sequence 6808 | R16295 | dbEST |
| Sequence 6809 | R16408 | dbEST |
| Sequence 6810 | R19453 | dbEST |
| Sequence 6811 | R19869 | dbEST |
| Sequence 6812 | R20063 | dbEST |
| Sequence 6813 | R24400 | dbEST |
| Sequence 6814 | R25464 | dbEST |
| Sequence 6815 | R33351 | dbEST |
| Sequence 6816 | R45686 | dbEST |
| Sequence 6817 | R53758 | dbEST |
| Sequence 6818 | R56382 | dbEST |
| Sequence 6819 | R61196 | dbEST |
| Sequence 6820 | R66162 | dbEST |
| Sequence 6821 | R70193 | dbEST |
| Sequence 6822 | R70570 | dbEST |
| Sequence 6823 | R72768 | dbEST |
| Sequence 6824 | R91218 | dbEST |
| Sequence 6825 | R94399 | dbEST |
| Sequence 6826 | R99755 | dbEST |
| Sequence 6827 | T08329 | dbEST |
| Sequence 6828 | T34459 | dbEST |
| Sequence 6829 | T35276 | dbEST |
| Sequence 6830 | T60601 | dbEST |
| Sequence 6831 | T67978 | dbEST |
| Sequence 6832 | T68894 | dbEST |
| Sequence 6833 | T71387 | dbEST |
| Sequence 6834 | T73262 | dbEST |
| Sequence 6835 | T81537 | dbEST |
| Sequence 6836 | W15207 | dbEST |
| Sequence 6837 | W15554 | dbEST |
| Sequence 6838 | W25142 | dbEST |
| Sequence 6839 | W26372 | dbEST |
| Sequence 6840 | W30832 | dbEST |
| Sequence 6841 | W38726 | dbEST |
| Sequence 6842 | W38956 | dbEST |
| Sequence 6843 | W42913 | dbEST |
| Sequence 6844 | W73290 | dbEST |
| Sequence 6845 | W76133 | dbEST |
| Sequence 6846 | W80496 | dbEST |
| Sequence 6847 | W81084 | dbEST |
| Sequence 6848 | N40101 | NUCPATENT |
| Sequence 6849 | N91258 | NUCPATENT |
| Sequence 6850 | Q24595 | NUCPATENT |
| Sequence 6851 | Q63815 | NUCPATENT |
| Sequence 6852 | Q97381 | NUCPATENT |
| Sequence 6853 | T74203 | NUCPATENT |
| Sequence 6854 | V07906 | NUCPATENT |
| Sequence 6855 | V30916 | NUCPATENT |
| Sequence 6856 | V34207 | NUCPATENT |
| Sequence 6857 | V34226 | NUCPATENT |
| Sequence 6858 | V34305 | NUCPATENT |
| Sequence 6859 | V35555 | NUCPATENT |
| Sequence 6860 | V43604 | NUCPATENT |
| Sequence 6861 | V58584 | NUCPATENT |
| Sequence 6862 | V58756 | NUCPATENT |
| Sequence 6863 | V58761 | NUCPATENT |
| Sequence 6864 | V62427 | NUCPATENT |
| Sequence 6865 | V69855 | NUCPATENT |
| Sequence 6866 | V70354 | NUCPATENT |
| Sequence 6867 | V72125 | NUCPATENT |
| Sequence 6868 | V72211 | NUCPATENT |
| Sequence 6869 | V84448 | NUCPATENT |
| Sequence 6870 | X27262 | NUCPATENT |
| Sequence 6871 | X27278 | NUCPATENT |
| Sequence 6872 | X30167 | NUCPATENT |
| Sequence 6873 | X36344 | NUCPATENT |
| Sequence 6874 | X36833 | NUCPATENT |
| Sequence 6875 | X37486 | NUCPATENT |
| Sequence 6876 | X39662 | NUCPATENT |
| Sequence 6877 | X40462 | NUCPATENT |
| Sequence 6878 | X51602 | NUCPATENT |
| Sequence 6879 | X97772 | NUCPATENT |
| Sequence 6880 | X97841 | NUCPATENT |
| Sequence 6881 | X97888 | NUCPATENT |
| Sequence 6882 | X98901 | NUCPATENT |
| Sequence 6883 | X99098 | NUCPATENT |
| Sequence 6884 | Z00842 | NUCPATENT |
| Sequence 6885 | Z06250 | NUCPATENT |
| Sequence 6886 | Z10658 | NUCPATENT |
| Sequence 6887 | Z15890 | NUCPATENT |
| Sequence 6888 | Z24862 | NUCPATENT |
| Sequence 6889 | AC29871 | PREPATNUC |
| Sequence 6890 | AC31081 | PREPATNUC |
| Sequence 6891 | AC33416 | PREPATNUC |
| Sequence 6892 | AC33722 | PREPATNUC |
| Sequence 6893 | AC33900 | PREPATNUC |
| Sequence 6894 | AC34235 | PREPATNUC |
| Sequence 6895 | AC34354 | PREPATNUC |
| Sequence 6896 | AC34866 | PREPATNUC |
| Sequence 6897 | AC34877 | PREPATNUC |
| Sequence 6898 | AB000468 | GENBANK |
| Sequence 6899 | AB004788 | GENBANK |
| Sequence 6900 | AB007893 | GENBANK |
| Sequence 6901 | AB009285 | GENBANK |
| Sequence 6902 | AB015644 | GENBANK |
| Sequence 6903 | AB016087 | GENBANK |
| Sequence 6904 | AF020351 | GENBANK |
| Sequence 6905 | AF035839 | GENBANK |
| Sequence 6906 | AF044957 | GENBANK |
| Sequence 6907 | AF048977 | GENBANK |
| Sequence 6908 | AF052138 | GENBANK |
| Sequence 6909 | AF054183 | GENBANK |
| Sequence 6910 | AF054987 | GENBANK |
| Sequence 6911 | AF055473 | GENBANK |
| Sequence 6912 | AF055474 | GENBANK |
| Sequence 6913 | AF067396 | GENBANK |
| Sequence 6914 | AF068117 | GENBANK |
| Sequence 6915 | AF068754 | GENBANK |
| Sequence 6916 | AF069301 | GENBANK |
| Sequence 6917 | AF070655 | GENBANK |
| Sequence 6918 | AF071172 | GENBANK |
| Sequence 6919 | AF071748 | GENBANK |
| Sequence 6920 | AF073298 | GENBANK |
| Sequence 6921 | AF077045 | GENBANK |
| Sequence 6922 | AF077202 | GENBANK |
| Sequence 6923 | AF083190 | GENBANK |
| Sequence 6924 | AF085844 | GENBANK |
| Sequence 6925 | AF086251 | GENBANK |
| Sequence 6926 | AF088035 | GENBANK |
| Sequence 6927 | AF091073 | GENBANK |
| Sequence 6928 | AF100759 | GENBANK |
| Sequence 6929 | AF101044 | GENBANK |
| Sequence 6930 | AF140598 | GENBANK |
| Sequence 6931 | AF147330 | GENBANK |
| Sequence 6932 | AF151823 | GENBANK |
| Sequence 6933 | AF151830 | GENBANK |
| Sequence 6934 | AF153603 | GENBANK |
| Sequence 6935 | AF153612 | GENBANK |
| Sequence 6936 | AF170708 | GENBANK |
| Sequence 6937 | AF176574 | GENBANK |
| Sequence 6938 | AJ007398 | GENBANK |
| Sequence 6939 | AL049981 | GENBANK |
| Sequence 6940 | AL050089 | GENBANK |
| Sequence 6941 | AL050291 | GENBANK |
| Sequence 6942 | AL080089 | GENBANK |
| Sequence 6943 | AL110185 | GENBANK |
| Sequence 6944 | AL110191 | GENBANK |
| Sequence 6945 | AL122063 | GENBANK |
| Sequence 6946 | D14697 | GENBANK |
| Sequence 6947 | D16469 | GENBANK |
| Sequence 6948 | D45248 | GENBANK |
| Sequence 6949 | D49396 | GENBANK |
| Sequence 6950 | D50371 | GENBANK |
| Sequence 6951 | D50525 | GENBANK |
| Sequence 6952 | D50916 | GENBANK |
| Sequence 6953 | D87666 | GENBANK |
| Sequence 6954 | D88153 | GENBANK |
| Sequence 6955 | E01915 | GENBANK |
| Sequence 6956 | J02642 | GENBANK |
| Sequence 6957 | J03779 | GENBANK |
| Sequence 6958 | J04208 | GENBANK |
| Sequence 6959 | L03558 | GENBANK |
| Sequence 6960 | L05093 | GENBANK |
| Sequence 6961 | L14778 | GENBANK |
| Sequence 6962 | L20422 | GENBANK |
| Sequence 6963 | M11147 | GENBANK |
| Sequence 6964 | M11560 | GENBANK |
| Sequence 6965 | M17885 | GENBANK |
| Sequence 6966 | M37583 | GENBANK |
| Sequence 6967 | M38188 | GENBANK |
| Sequence 6968 | M64241 | GENBANK |
| Sequence 6969 | U12979 | GENBANK |
| Sequence 6970 | U17104 | GENBANK |
| Sequence 6971 | U17899 | GENBANK |
| Sequence 6972 | U19143 | GENBANK |
| Sequence 6973 | U19144 | GENBANK |
| Sequence 6974 | U22431 | GENBANK |
| Sequence 6975 | U73824 | GENBANK |
| Sequence 6976 | U75330 | GENBANK |
| Sequence 6977 | X07767 | GENBANK |
| Sequence 6978 | X62048 | GENBANK |
| Sequence 6979 | X63423 | GENBANK |
| Sequence 6980 | X75535 | GENBANK |
| Sequence 6981 | X89673 | GENBANK |
| Sequence 6982 | X99585 | GENBANK |
| Sequence 6983 | Z11692 | GENBANK |
| Sequence 6984 | Z19002 | GENBANK |
| Sequence 6985 | Z47087 | GENBANK |
| Sequence 6986 | Z48054 | GENBANK |
| Sequence 6987 | AA001699 | dbEST |
| Sequence 6988 | AA004206 | dbEST |
| Sequence 6989 | AA005403 | dbEST |
| Sequence 6990 | AA009849 | dbEST |
| Sequence 6991 | AA010129 | dbEST |
| Sequence 6992 | AA016205 | dbEST |
| Sequence 6993 | AA018050 | dbEST |
| Sequence 6994 | AA025694 | dbEST |
| Sequence 6995 | AA027090 | dbEST |
| Sequence 6996 | AA029566 | dbEST |
| Sequence 6997 | AA031624 | dbEST |
| Sequence 6998 | AA031795 | dbEST |
| Sequence 6999 | AA033537 | dbEST |
| Sequence 7000 | AA039274 | dbEST |
| Sequence 7001 | AA039506 | dbEST |
| Sequence 7002 | AA039734 | dbEST |
| Sequence 7003 | AA039750 | dbEST |
| Sequence 7004 | AA039807 | dbEST |
| Sequence 7005 | AA040499 | dbEST |
| Sequence 7006 | AA040625 | dbEST |
| Sequence 7007 | AA042813 | dbEST |
| Sequence 7008 | AA044697 | dbEST |
| Sequence 7009 | AA045753 | dbEST |
| Sequence 7010 | AA045879 | dbEST |
| Sequence 7011 | AA057541 | dbEST |
| Sequence 7012 | AA062804 | dbEST |
| Sequence 7013 | AA062928 | dbEST |
| Sequence 7014 | AA064627 | dbEST |
| Sequence 7015 | AA070911 | dbEST |
| Sequence 7016 | AA076077 | dbEST |
| Sequence 7017 | AA076593 | dbEST |
| Sequence 7018 | AA081815 | dbEST |
| Sequence 7019 | AA082502 | dbEST |
| Sequence 7020 | AA083481 | dbEST |
| Sequence 7021 | AA083794 | dbEST |
| Sequence 7022 | AA083868 | dbEST |
| Sequence 7023 | AA085603 | dbEST |
| Sequence 7024 | AA085824 | dbEST |
| Sequence 7025 | AA094583 | dbEST |
| Sequence 7026 | AA095753 | dbEST |
| Sequence 7027 | AA098801 | dbEST |
| Sequence 7028 | AA099023 | dbEST |
| Sequence 7029 | AA100987 | dbEST |
| Sequence 7030 | AA121390 | dbEST |
| Sequence 7031 | AA127493 | dbEST |
| Sequence 7032 | AA128952 | dbEST |
| Sequence 7033 | AA129726 | dbEST |
| Sequence 7034 | AA130873 | dbEST |
| Sequence 7035 | AA132219 | dbEST |
| Sequence 7036 | AA132574 | dbEST |
| Sequence 7037 | AA132690 | dbEST |
| Sequence 7038 | AA133228 | dbEST |
| Sequence 7039 | AA133266 | dbEST |
| Sequence 7040 | AA135049 | dbEST |
| Sequence 7041 | AA136150 | dbEST |
| Sequence 7042 | AA136296 | dbEST |
| Sequence 7043 | AA147986 | dbEST |
| Sequence 7044 | AA149080 | dbEST |
| Sequence 7045 | AA150156 | dbEST |
| Sequence 7046 | AA150267 | dbEST |
| Sequence 7047 | AA150369 | dbEST |
| Sequence 7048 | AA150839 | dbEST |
| Sequence 7049 | AA150875 | dbEST |
| Sequence 7050 | AA152408 | dbEST |
| Sequence 7051 | AA156743 | dbEST |
| Sequence 7052 | AA157816 | dbEST |
| Sequence 7053 | AA158098 | dbEST |
| Sequence 7054 | AA159044 | dbEST |
| Sequence 7055 | AA160380 | dbEST |
| Sequence 7056 | AA164405 | dbEST |
| Sequence 7057 | AA165117 | dbEST |
| Sequence 7058 | AA166853 | dbEST |
| Sequence 7059 | AA167011 | dbEST |
| Sequence 7060 | AA169493 | dbEST |
| Sequence 7061 | AA171618 | dbEST |
| Sequence 7062 | AA171706 | dbEST |
| Sequence 7063 | AA177062 | dbEST |
| Sequence 7064 | AA180137 | dbEST |
| Sequence 7065 | AA180817 | dbEST |
| Sequence 7066 | AA191045 | dbEST |
| Sequence 7067 | AA194429 | dbEST |
| Sequence 7068 | AA194535 | dbEST |
| Sequence 7069 | AA195470 | dbEST |
| Sequence 7070 | AA203462 | dbEST |
| Sequence 7071 | AA204856 | dbEST |
| Sequence 7072 | AA205858 | dbEST |
| Sequence 7073 | AA216133 | dbEST |
| Sequence 7074 | AA216211 | dbEST |
| Sequence 7075 | AA219582 | dbEST |
| Sequence 7076 | AA226340 | dbEST |
| Sequence 7077 | AA227105 | dbEST |
| Sequence 7078 | AA227695 | dbEST |
| Sequence 7079 | AA229607 | dbEST |
| Sequence 7080 | AA229611 | dbEST |
| Sequence 7081 | AA232686 | dbEST |
| Sequence 7082 | AA233053 | dbEST |
| Sequence 7083 | AA253254 | dbEST |
| Sequence 7084 | AA255502 | dbEST |
| Sequence 7085 | AA255529 | dbEST |
| Sequence 7086 | AA256507 | dbEST |
| Sequence 7087 | AA280381 | dbEST |
| Sequence 7088 | AA282837 | dbEST |
| Sequence 7089 | AA284506 | dbEST |
| Sequence 7090 | AA285089 | dbEST |
| Sequence 7091 | AA285290 | dbEST |
| Sequence 7092 | AA287721 | dbEST |
| Sequence 7093 | AA292273 | dbEST |
| Sequence 7094 | AA296152 | dbEST |
| Sequence 7095 | AA296237 | dbEST |
| Sequence 7096 | AA301795 | dbEST |
| Sequence 7097 | AA306372 | dbEST |
| Sequence 7098 | AA306982 | dbEST |
| Sequence 7099 | AA307814 | dbEST |
| Sequence 7100 | AA307908 | dbEST |
| Sequence 7101 | AA313647 | dbEST |
| Sequence 7102 | AA313693 | dbEST |
| Sequence 7103 | AA314391 | dbEST |
| Sequence 7104 | AA316788 | dbEST |
| Sequence 7105 | AA316962 | dbEST |
| Sequence 7106 | AA318100 | dbEST |
| Sequence 7107 | AA318712 | dbEST |
| Sequence 7108 | AA325594 | dbEST |
| Sequence 7109 | AA328061 | dbEST |
| Sequence 7110 | AA329553 | dbEST |
| Sequence 7111 | AA351201 | dbEST |
| Sequence 7112 | AA354420 | dbEST |
| Sequence 7113 | AA380769 | dbEST |
| Sequence 7114 | AA393336 | dbEST |
| Sequence 7115 | AA398256 | dbEST |
| Sequence 7116 | AA399238 | dbEST |
| Sequence 7117 | AA400658 | dbEST |
| Sequence 7118 | AA401237 | dbEST |
| Sequence 7119 | AA402330 | dbEST |
| Sequence 7120 | AA404535 | dbEST |
| Sequence 7121 | AA405929 | dbEST |
| Sequence 7122 | AA406122 | dbEST |
| Sequence 7123 | AA406404 | dbEST |
| Sequence 7124 | AA406565 | dbEST |
| Sequence 7125 | AA410640 | dbEST |
| Sequence 7126 | AA412229 | dbEST |
| Sequence 7127 | AA420563 | dbEST |
| Sequence 7128 | AA424175 | dbEST |
| Sequence 7129 | AA425389 | dbEST |
| Sequence 7130 | AA429036 | dbEST |
| Sequence 7131 | AA429457 | dbEST |
| Sequence 7132 | AA431401 | dbEST |
| Sequence 7133 | AA435877 | dbEST |
| Sequence 7134 | AA443357 | dbEST |
| Sequence 7135 | AA447559 | dbEST |
| Sequence 7136 | AA448542 | dbEST |
| Sequence 7137 | AA449663 | dbEST |
| Sequence 7138 | AA449891 | dbEST |
| Sequence 7139 | AA452722 | dbEST |
| Sequence 7140 | AA459869 | dbEST |
| Sequence 7141 | AA460777 | dbEST |
| Sequence 7142 | AA469432 | dbEST |
| Sequence 7143 | AA470662 | dbEST |
| Sequence 7144 | AA470829 | dbEST |
| Sequence 7145 | AA477597 | dbEST |
| Sequence 7146 | AA479363 | dbEST |
| Sequence 7147 | AA480202 | dbEST |
| Sequence 7148 | AA480226 | dbEST |
| Sequence 7149 | AA480863 | dbEST |
| Sequence 7150 | AA483013 | dbEST |
| Sequence 7151 | AA483289 | dbEST |
| Sequence 7152 | AA483857 | dbEST |
| Sequence 7153 | AA485065 | dbEST |
| Sequence 7154 | AA487463 | dbEST |
| Sequence 7155 | AA489074 | dbEST |
| Sequence 7156 | AA492038 | dbEST |
| Sequence 7157 | AA501447 | dbEST |
| Sequence 7158 | AA502178 | dbEST |
| Sequence 7159 | AA502730 | dbEST |
| Sequence 7160 | AA504514 | dbEST |
| Sequence 7161 | AA504622 | dbEST |
| Sequence 7162 | AA506953 | dbEST |
| Sequence 7163 | AA508507 | dbEST |
| Sequence 7164 | AA512975 | dbEST |
| Sequence 7165 | AA514324 | dbEST |
| Sequence 7166 | AA514474 | dbEST |
| Sequence 7167 | AA521013 | dbEST |
| Sequence 7168 | AA522638 | dbEST |
| Sequence 7169 | AA522746 | dbEST |
| Sequence 7170 | AA522849 | dbEST |
| Sequence 7171 | AA523503 | dbEST |
| Sequence 7172 | AA525960 | dbEST |
| Sequence 7173 | AA526187 | dbEST |
| Sequence 7174 | AA526243 | dbEST |
| Sequence 7175 | AA526893 | dbEST |
| Sequence 7176 | AA531582 | dbEST |
| Sequence 7177 | AA533372 | dbEST |
| Sequence 7178 | AA533386 | dbEST |
| Sequence 7179 | AA534827 | dbEST |
| Sequence 7180 | AA535755 | dbEST |
| Sequence 7181 | AA548472 | dbEST |
| Sequence 7182 | AA554018 | dbEST |
| Sequence 7183 | AA555194 | dbEST |
| Sequence 7184 | AA557408 | dbEST |
| Sequence 7185 | AA564238 | dbEST |
| Sequence 7186 | AA565272 | dbEST |
| Sequence 7187 | AA568518 | dbEST |
| Sequence 7188 | AA568538 | dbEST |
| Sequence 7189 | AA569493 | dbEST |
| Sequence 7190 | AA574227 | dbEST |
| Sequence 7191 | AA577490 | dbEST |
| Sequence 7192 | AA579160 | dbEST |
| Sequence 7193 | AA579617 | dbEST |
| Sequence 7194 | AA581892 | dbEST |
| Sequence 7195 | AA582930 | dbEST |
| Sequence 7196 | AA584026 | dbEST |
| Sequence 7197 | AA585278 | dbEST |
| Sequence 7198 | AA585430 | dbEST |
| Sequence 7199 | AA586522 | dbEST |
| Sequence 7200 | AA587348 | dbEST |
| Sequence 7201 | AA592908 | dbEST |
| Sequence 7202 | AA594976 | dbEST |
| Sequence 7203 | AA595339 | dbEST |
| Sequence 7204 | AA595581 | dbEST |
| Sequence 7205 | AA599534 | dbEST |
| Sequence 7206 | AA599711 | dbEST |
| Sequence 7207 | AA600060 | dbEST |
| Sequence 7208 | AA602634 | dbEST |
| Sequence 7209 | AA602758 | dbEST |
| Sequence 7210 | AA604815 | dbEST |
| Sequence 7211 | AA613314 | dbEST |
| Sequence 7212 | AA617696 | dbEST |
| Sequence 7213 | AA618183 | dbEST |
| Sequence 7214 | AA618435 | dbEST |
| Sequence 7215 | AA621730 | dbEST |
| Sequence 7216 | AA625270 | dbEST |
| Sequence 7217 | AA629269 | dbEST |
| Sequence 7218 | AA631009 | dbEST |
| Sequence 7219 | AA631608 | dbEST |
| Sequence 7220 | AA632154 | dbEST |
| Sequence 7221 | AA635393 | dbEST |
| Sequence 7222 | AA643685 | dbEST |
| Sequence 7223 | AA644700 | dbEST |
| Sequence 7224 | AA659764 | dbEST |
| Sequence 7225 | AA662406 | dbEST |
| Sequence 7226 | AA678414 | dbEST |
| Sequence 7227 | AA683075 | dbEST |
| Sequence 7228 | AA687675 | dbEST |
| Sequence 7229 | AA689392 | dbEST |
| Sequence 7230 | AA694271 | dbEST |
| Sequence 7231 | AA699674 | dbEST |
| Sequence 7232 | AA700599 | dbEST |
| Sequence 7233 | AA701980 | dbEST |
| Sequence 7234 | AA706018 | dbEST |
| Sequence 7235 | AA706860 | dbEST |
| Sequence 7236 | AA715000 | dbEST |
| Sequence 7237 | AA719797 | dbEST |
| Sequence 7238 | AA723045 | dbEST |
| Sequence 7239 | AA723521 | dbEST |
| Sequence 7240 | AA723584 | dbEST |
| Sequence 7241 | AA731872 | dbEST |
| Sequence 7242 | AA737421 | dbEST |
| Sequence 7243 | AA737949 | dbEST |
| Sequence 7244 | AA740610 | dbEST |
| Sequence 7245 | AA746487 | dbEST |
| Sequence 7246 | AA746824 | dbEST |
| Sequence 7247 | AA747091 | dbEST |
| Sequence 7248 | AA767779 | dbEST |
| Sequence 7249 | AA768109 | dbEST |
| Sequence 7250 | AA769865 | dbEST |
| Sequence 7251 | AA779278 | dbEST |
| Sequence 7252 | AA780301 | dbEST |
| Sequence 7253 | AA782769 | dbEST |
| Sequence 7254 | AA804740 | dbEST |
| Sequence 7255 | AA805604 | dbEST |
| Sequence 7256 | AA808349 | dbEST |
| Sequence 7257 | AA808582 | dbEST |
| Sequence 7258 | AA813708 | dbEST |
| Sequence 7259 | AA825194 | dbEST |
| Sequence 7260 | AA827738 | dbEST |
| Sequence 7261 | AA827816 | dbEST |
| Sequence 7262 | AA829850 | dbEST |
| Sequence 7263 | AA829973 | dbEST |
| Sequence 7264 | AA830635 | dbEST |
| Sequence 7265 | AA835708 | dbEST |
| Sequence 7266 | AA836233 | dbEST |
| Sequence 7267 | AA836306 | dbEST |
| Sequence 7268 | AA836962 | dbEST |
| Sequence 7269 | AA837401 | dbEST |
| Sequence 7270 | AA845575 | dbEST |
| Sequence 7271 | AA846308 | dbEST |
| Sequence 7272 | AA846626 | dbEST |
| Sequence 7273 | AA847348 | dbEST |
| Sequence 7274 | AA852908 | dbEST |
| Sequence 7275 | AA864406 | dbEST |
| Sequence 7276 | AA865913 | dbEST |
| Sequence 7277 | AA866035 | dbEST |
| Sequence 7278 | AA872969 | dbEST |
| Sequence 7279 | AA876384 | dbEST |
| Sequence 7280 | AA876730 | dbEST |
| Sequence 7281 | AA886406 | dbEST |
| Sequence 7282 | AA887224 | dbEST |
| Sequence 7283 | AA894408 | dbEST |
| Sequence 7284 | AA912233 | dbEST |
| Sequence 7285 | AA916635 | dbEST |
| Sequence 7286 | AA936792 | dbEST |
| Sequence 7287 | AA960820 | dbEST |
| Sequence 7288 | AA974826 | dbEST |
| Sequence 7289 | AA994034 | dbEST |
| Sequence 7290 | AA994694 | dbEST |
| Sequence 7291 | AI002321 | dbEST |
| Sequence 7292 | AI016106 | dbEST |
| Sequence 7293 | AI017779 | dbEST |
| Sequence 7294 | AI017809 | dbEST |
| Sequence 7295 | AI018161 | dbEST |
| Sequence 7296 | AI018686 | dbEST |
| Sequence 7297 | AI028107 | dbEST |
| Sequence 7298 | AI032779 | dbEST |
| Sequence 7299 | AI033863 | dbEST |
| Sequence 7300 | AI039433 | dbEST |
| Sequence 7301 | AI039804 | dbEST |
| Sequence 7302 | AI041630 | dbEST |
| Sequence 7303 | AI042066 | dbEST |
| Sequence 7304 | AI042354 | dbEST |
| Sequence 7305 | AI051210 | dbEST |
| Sequence 7306 | AI051983 | dbEST |
| Sequence 7307 | AI051994 | dbEST |
| Sequence 7308 | AI053744 | dbEST |
| Sequence 7309 | AI054344 | dbEST |
| Sequence 7310 | AI056927 | dbEST |
| Sequence 7311 | AI057585 | dbEST |
| Sequence 7312 | AI066753 | dbEST |
| Sequence 7313 | AI074107 | dbEST |
| Sequence 7314 | AI076593 | dbEST |
| Sequence 7315 | AI077317 | dbEST |
| Sequence 7316 | AI081842 | dbEST |
| Sequence 7317 | AI081918 | dbEST |
| Sequence 7318 | AI085195 | dbEST |
| Sequence 7319 | AI087077 | dbEST |
| Sequence 7320 | AI087287 | dbEST |
| Sequence 7321 | AI091935 | dbEST |
| Sequence 7322 | AI093409 | dbEST |
| Sequence 7323 | AI097545 | dbEST |
| Sequence 7324 | AI114631 | dbEST |
| Sequence 7325 | AI123229 | dbEST |
| Sequence 7326 | AI125607 | dbEST |
| Sequence 7327 | AI131097 | dbEST |
| Sequence 7328 | AI138629 | dbEST |
| Sequence 7329 | AI139904 | dbEST |
| Sequence 7330 | AI144216 | dbEST |
| Sequence 7331 | AI147191 | dbEST |
| Sequence 7332 | AI174867 | dbEST |
| Sequence 7333 | AI183897 | dbEST |
| Sequence 7334 | AI184113 | dbEST |
| Sequence 7335 | AI208430 | dbEST |
| Sequence 7336 | AI214050 | dbEST |
| Sequence 7337 | AI217019 | dbEST |
| Sequence 7338 | AI217557 | dbEST |
| Sequence 7339 | AI222506 | dbEST |
| Sequence 7340 | AI240543 | dbEST |
| Sequence 7341 | AI241893 | dbEST |
| Sequence 7342 | AI246173 | dbEST |
| Sequence 7343 | AI249257 | dbEST |
| Sequence 7344 | AI249877 | dbEST |
| Sequence 7345 | AI252562 | dbEST |
| Sequence 7346 | AI262104 | dbEST |
| Sequence 7347 | AI262460 | dbEST |
| Sequence 7348 | AI263731 | dbEST |
| Sequence 7349 | AI266643 | dbEST |
| Sequence 7350 | AI271269 | dbEST |
| Sequence 7351 | AI275175 | dbEST |
| Sequence 7352 | AI280561 | dbEST |
| Sequence 7353 | AI281777 | dbEST |
| Sequence 7354 | AI283385 | dbEST |
| Sequence 7355 | AI301933 | dbEST |
| Sequence 7356 | AI302526 | dbEST |
| Sequence 7357 | AI305530 | dbEST |
| Sequence 7358 | AI306421 | dbEST |
| Sequence 7359 | AI309025 | dbEST |
| Sequence 7360 | AI309843 | dbEST |
| Sequence 7361 | AI310484 | dbEST |
| Sequence 7362 | AI338222 | dbEST |
| Sequence 7363 | AI340533 | dbEST |
| Sequence 7364 | AI340627 | dbEST |
| Sequence 7365 | AI345347 | dbEST |
| Sequence 7366 | AI345370 | dbEST |
| Sequence 7367 | AI345478 | dbEST |
| Sequence 7368 | AI347461 | dbEST |
| Sequence 7369 | AI349256 | dbEST |
| Sequence 7370 | AI357779 | dbEST |
| Sequence 7371 | AI360123 | dbEST |
| Sequence 7372 | AI363118 | dbEST |
| Sequence 7373 | AI364592 | dbEST |
| Sequence 7374 | AI367730 | dbEST |
| Sequence 7375 | AI373565 | dbEST |
| Sequence 7376 | AI375751 | dbEST |
| Sequence 7377 | AI394420 | dbEST |
| Sequence 7378 | AI394469 | dbEST |
| Sequence 7379 | AI400282 | dbEST |
| Sequence 7380 | AI401513 | dbEST |
| Sequence 7381 | AI420373 | dbEST |
| Sequence 7382 | AI421290 | dbEST |
| Sequence 7383 | AI421401 | dbEST |
| Sequence 7384 | AI431327 | dbEST |
| Sequence 7385 | AI431923 | dbEST |
| Sequence 7386 | AI434109 | dbEST |
| Sequence 7387 | AI434949 | dbEST |
| Sequence 7388 | AI439452 | dbEST |
| Sequence 7389 | AI440238 | dbEST |
| Sequence 7390 | AI446003 | dbEST |
| Sequence 7391 | AI446426 | dbEST |
| Sequence 7392 | AI453477 | dbEST |
| Sequence 7393 | AI457107 | dbEST |
| Sequence 7394 | AI457410 | dbEST |
| Sequence 7395 | AI458799 | dbEST |
| Sequence 7396 | AI472566 | dbEST |
| Sequence 7397 | AI476075 | dbEST |
| Sequence 7398 | AI492953 | dbEST |
| Sequence 7399 | AI497636 | dbEST |
| Sequence 7400 | AI499584 | dbEST |
| Sequence 7401 | AI523964 | dbEST |
| Sequence 7402 | AI524304 | dbEST |
| Sequence 7403 | AI537244 | dbEST |
| Sequence 7404 | AI538342 | dbEST |
| Sequence 7405 | AI557082 | dbEST |
| Sequence 7406 | AI560184 | dbEST |
| Sequence 7407 | AI560756 | dbEST |
| Sequence 7408 | AI570521 | dbEST |
| Sequence 7409 | AI572096 | dbEST |
| Sequence 7410 | AI573218 | dbEST |
| Sequence 7411 | AI582912 | dbEST |
| Sequence 7412 | AI583257 | dbEST |
| Sequence 7413 | AI611738 | dbEST |
| Sequence 7414 | AI625204 | dbEST |
| Sequence 7415 | AI630345 | dbEST |
| Sequence 7416 | AI630891 | dbEST |
| Sequence 7417 | AI635478 | dbEST |
| Sequence 7418 | AI636626 | dbEST |
| Sequence 7419 | AI637584 | dbEST |
| Sequence 7420 | AI653541 | dbEST |
| Sequence 7421 | AI656402 | dbEST |
| Sequence 7422 | AI659386 | dbEST |
| Sequence 7423 | AI670009 | dbEST |
| Sequence 7424 | AI671642 | dbEST |
| Sequence 7425 | AI687362 | dbEST |
| Sequence 7426 | AI696693 | dbEST |
| Sequence 7427 | AI698092 | dbEST |
| Sequence 7428 | AI720132 | dbEST |
| Sequence 7429 | AI741446 | dbEST |
| Sequence 7430 | AI741861 | dbEST |
| Sequence 7431 | AI745218 | dbEST |
| Sequence 7432 | AI753459 | dbEST |
| Sequence 7433 | AI796562 | dbEST |
| Sequence 7434 | AI799699 | dbEST |
| Sequence 7435 | AI801589 | dbEST |
| Sequence 7436 | AI802542 | dbEST |
| Sequence 7437 | AI811144 | dbEST |
| Sequence 7438 | AI826427 | dbEST |
| Sequence 7439 | AI827129 | dbEST |
| Sequence 7440 | AI827550 | dbEST |
| Sequence 7441 | AI832609 | dbEST |
| Sequence 7442 | AI866551 | dbEST |
| Sequence 7443 | AI913323 | dbEST |
| Sequence 7444 | AI916374 | dbEST |
| Sequence 7445 | AI916419 | dbEST |
| Sequence 7446 | AI921254 | dbEST |
| Sequence 7447 | AI922114 | dbEST |
| Sequence 7448 | AI923485 | dbEST |
| Sequence 7449 | AI928889 | dbEST |
| Sequence 7450 | AI932951 | dbEST |
| Sequence 7451 | AI937150 | dbEST |
| Sequence 7452 | AI954406 | dbEST |
| Sequence 7453 | AI961592 | dbEST |
| Sequence 7454 | AI968311 | dbEST |
| Sequence 7455 | AL036165 | dbEST |
| Sequence 7456 | AL036575 | dbEST |
| Sequence 7457 | AL037646 | dbEST |
| Sequence 7458 | AL043071 | dbEST |
| Sequence 7459 | AL045040 | dbEST |
| Sequence 7460 | AL045500 | dbEST |
| Sequence 7461 | AL046648 | dbEST |
| Sequence 7462 | AL110384 | dbEST |
| Sequence 7463 | AL119319 | dbEST |
| Sequence 7464 | AW009700 | dbEST |
| Sequence 7465 | AW009886 | dbEST |
| Sequence 7466 | AW015094 | dbEST |
| Sequence 7467 | AW016443 | dbEST |
| Sequence 7468 | AW016546 | dbEST |
| Sequence 7469 | AW020245 | dbEST |
| Sequence 7470 | AW021402 | dbEST |
| Sequence 7471 | AW023884 | dbEST |
| Sequence 7472 | AW025130 | dbEST |
| Sequence 7473 | AW043908 | dbEST |
| Sequence 7474 | AW075324 | dbEST |
| Sequence 7475 | AW079132 | dbEST |
| Sequence 7476 | AW080456 | dbEST |
| Sequence 7477 | AW084350 | dbEST |
| Sequence 7478 | AW084663 | dbEST |
| Sequence 7479 | AW117855 | dbEST |
| Sequence 7480 | AW130776 | dbEST |
| Sequence 7481 | AW149504 | dbEST |
| Sequence 7482 | AW161853 | dbEST |
| Sequence 7483 | AW167569 | dbEST |
| Sequence 7484 | AW169156 | dbEST |
| Sequence 7485 | AW183848 | dbEST |
| Sequence 7486 | D52802 | dbEST |
| Sequence 7487 | D55565 | dbEST |
| Sequence 7488 | F28764 | dbEST |
| Sequence 7489 | H47715 | dbEST |
| Sequence 7490 | H48257 | dbEST |
| Sequence 7491 | H69070 | dbEST |
| Sequence 7492 | H93650 | dbEST |
| Sequence 7493 | N30570 | dbEST |
| Sequence 7494 | N31301 | dbEST |
| Sequence 7495 | N75979 | dbEST |
| Sequence 7496 | R48046 | dbEST |
| Sequence 7497 | W05661 | dbEST |
| Sequence 7498 | W35236 | dbEST |
| Sequence 7499 | W37602 | dbEST |
| Sequence 7500 | W73817 | dbEST |
| Sequence 7501 | W79668 | dbEST |
| Sequence 7502 | W79717 | dbEST |
| Sequence 7503 | Q65520 | NUCPATENT |
| Sequence 7504 | V05728 | NUCPATENT |
| Sequence 7505 | V63195 | NUCPATENT |
| Sequence 7506 | X00656 | NUCPATENT |
| Sequence 7507 | X08687 | NUCPATENT |
| Sequence 7508 | X24921 | NUCPATENT |
| Sequence 7509 | X27427 | NUCPATENT |
| Sequence 7510 | X85038 | NUCPATENT |
| Sequence 7511 | X89652 | NUCPATENT |

TABLE 3-2
Sequence 1240
CTACTTAGGGCGAATTGGAGCTCCCCGCGGTGGCGGCCGAGGTACGCGGGGAGAGTGTTC
CAGACAGAAGAAATAGCAAGTGCCGAGAAGCTGGCATCAGAAAAACAGAGGGGAGATTTG
TGTGGCTGCAGCCGAGGGAGACCAGGAAGATCTGCATGGTGGGAAGGACCTGATGATACA
GAGGTGAGAAATAAGAAAGGCTGCTGACTTTACCATCTGAGGCCACACATCTGCTGAAAT
GGAGATAATTAACATCACTAGAAACAGCAAGATGACAATATAATGTCTAAGTAGTGACAT
GTTTTTGCACATTTCCAGCCCCTTTAAATNTCCACACACACAGGAAGCACAAAAGGAAGC
ACAGAGATCCTGGGAGAAATGCCCGGCCCGCTCTA
Sequence 2802
CCGCGGTGGCGGCCGAGGTNCCAANGTCAAGTCACCATAATGTNTTTNACCTGTTTCACA
ATGTTCTACATTATCGTGGAAATGTTGACAAGGTCTCNAGCAAAGACTGGATCTNCANTG
GNTTTAAAATACAATNNTCCATTTCGGAAATTGANGGAAAAAGATCTTTNNCAANCTGGA
AGAGTCTTCTTGTAAATAGGAATCACNGGGCTACACCTGCTGACGACCTTGGGTTTN
Sequence 2803
TTAGGGCGAATTGGAGCTCCCCGCGGTGGCGGCCGAGGTACTATATGGACACCAACATGG
AGCANAAAATGAGGGCAGCAGTGTCCAATCTCATTCCAGGTGAGAAGTTGCACAGTGTCC
AATAGGTGCATACATCTCCTTAGTAAGTAGTTGTGATTAACAATGAAATAGAAATGAAAA
ATATATTTTTTTATTTATGTGTATTATATTTTTCAAGCAGCTAATAAGTTGGTAGGACAT
AATATTTAATTCGTTGGGGACCTAATTATTTATAAATTGAATGGTTAGATATTTCTTTTG
GCCTAAGCCACCATGAAAAAATCACTGAGGCGCTAAGGGAAACATGAACTAAGAAGCCCT
TCTGAGTCTCTGTTTTCTCAACTGTAAGATGAANAAACCTGCTCCACCTTCCTCTTGCGA
TTGGTGAACGGCAGTTTGAGATACTGCGCAGAATGGACCTTAT
Sequence 2804
GGAGCTCCCCGCGGTGGCGGCCGAGGTACGCGGGGGTTGTATGTGGGTTGGCATTCTTGC
ATGATGGGAGTGGCCACCTGCTTTCATATTCTGAAGTCAGAGTGTTCCAGACAGAAGAAA
TAGCAAGTGCCGAGAAGCTGGCATCAGAAAAACAGAGGGGAGATTTGTGTGGCTGCAGCC
GAGGGAGACCAGGAAGATCTGCATGGTGGGAAGGACCTGATGATACAGAGGTGAGAAATA
AGAAAGGCTGCTGACTTTACCATCTGAGGGCACACATCTGCTGAAATGGAGATAATTAAC
ATCACTAGAAACAGCAAGATGACAATATAATGTCTAANTAGTGACATGTTTTTTGCACAT
TTCCAGCCCCTTTAAATATCGACACACACAGGAAGCACAAAAGGAAGCACAGAGATCCCT
GGGAGAAATGCCCG
Sequence 2805
CTATAGGGCGAATTGGAGCTCCCCGCGGTGGCGGCCGCCCGGGCATGGTACGCGGGGAGG
TGTTGTTCCGTGAATCAGCACATCAATTGCAGCATTGTGGCTACCAGGGGGTCAGGATGC
GGNCGGTGGAGCCCTCTGGCCTTTGTGTGGTAGCCGAGGACTCTGTGTCAGCTGACCGTT
TTCCGGNAAACTTTCGTGCGAGACTCACATCTTGGAAATTCAAATACTCAATANCTCTCG
AATTCTAGGAATCTTGAGAAGAGGCCTGGATTAANGATTCANACATGGGCCCTNANATNG
NTATGGCATTGCTGGTTCTACCAACGTGACAG
Sequence 4710
CCGGGCAGGTACCAGAACTTCAGCAAAGGAAGCAGACACAGGTCGGAGGAAGCCTACATA
GACCCCATTGCCATGGAGTATTACAACTGGGGGCGGTTCTCGAAGCCCCCAGAAGATGAT
GATGCCAATTCCTACGAGAATGTGCTCATTTGCAAGCAGAAAACCACAGAGACAGGTGCC
CAGCAGGAGGGCATAGGTGGCCTCTGCAGAGGGGACCTCAGCCTGTCACTGGCCCTGAAG
ACTGGCCCCACTTCTGGTCTCTGTCCCCCTGCCTCCCCGGAAGAAGATGAGGAATCTGAG
GATTATCAGAACTCAGCATCCATCCATCAGTGGCACGAGTCCAGGAAG
Sequence 4711
TTAGGGCGAATTGGAGCTCCCCGCGGTGGCGGCCGAGGTACTTTTTTTTTTTTTTTTTTT
TTTTGGACCTTGGGTATCTTCTGCAGGTCAAGCCATCCTGACCACTCTGAGAACTTACCC
AGGGTGACTCANCTGTCACTCCAGGTCACTCATAAGCCCAACGGGTAGCTCCACCCGTGG
ACAGCTGG
Sequence 4712
CGCGGTGGCGGCCCGCCCGGGCAGGTACTTTTTTTTTTTTTTTTTTTTTTTTTTTTTAAG
GGAACGTGAATTTTAATGAGGGGGCANACCGAGGAGGTGGTGGCTGCCCGGAGATCAGGG
CCAGGCTGTGCTANATGGCNCCTGGAAGGGGGGTCACCCAAGTCTCCCTGCTATCATTTC
AGGAGGCCGACCCAAGTCTCCCTGCTGTCATTTCAGGAGGCCGAATTTTTTCCCGATCCC
ANAGAAGGTGTCAAAGGCCTGGTTAGCANTCTTGTCGATGGTTTCCTGGGTGGTCTTGGC
CANCTGGTCCATGGCTTTCTGCCCCGCCTCTGTGGCCTGGTCCACCACTTGCTGANCTGC
CGCTCCGGCCCGCTNACACGGCTTCCTGGGCGGTCCCCTCCACCTGTTGCTTCAGGTCCT
GCAAGCCCTTGCTTGCCATGGCTTCGGGGTATCTGTGGAGTCNTCAAGANCANCTGGAGC
CACNTTGGATCCTNCCCAAAGTGGCCCCCGCGTACCTTGGCCCGTNTAAAACTATGTGGA
ATCCCCCCGGCCTGCANGAATTCCATATCAAAGCTTATNGATACCCGGCGACCCTCGAGG
GGG
Sequence 4713
CCGCGGTGGCGGCCGAGGTACGCGGGGTCATTACTACAGGAAAAACTGTTCTCTTCTGTG
GCACAGAGAAGCCTGCTTCAAAGCAGAAGTAGCAGTTCCGGAGTCCAGCTGGCTAAAACT
CATCCCAGAGGATAATGGCAACCCATGCCTTAGAAATCGCTGGGCTGTTTCTTGGTGGTG
TTGGAATGGTGGGCACAGTGGCTGTCACTGTCATGCCTCAGTGGAGAGTGTCGGCCTTCA
TTGAAAACAACATCGTGGTTTTTGAAAACTTCTGGGAAGGACTGTGGATGAATTGCGTGA
GGCAGGCTAACATCAGGATGCAGTTGCAAAATCTATGATTCCCTGCTGGCTCTTTCTCCG
GACCTACAGGCAGCCAGAGGACTGATGTTGCGCTGCTTCCGTGATGTCCTTCTTGGCTTT
CATGATGGC
Sequence 4714
CGCTGATGTTGGCCANGCGAGAGGGCCACGTTCTCCTGAGTGTTCAGAAGGGTCTTACAG
ACGTCCAGGGGGGTCGTGGCGGCCGCGGCGAGGGCCCCGGCCAGCCCGCCTGAGATGATG
TGGGACTGCGGGTTGTAGGTCCGGTGGGGGTTGACCTGCTCCTGCAGGAACTCATAGGTG
ATGAAGTGGATGGACTGGAAGGGGATGTTCATGGTCAGCTGCGTGGTGTAGCTCCGGTAG
AAGGCCCCCAACCCCTNGGTCCTCCACACCGTCCGGATGCAACTGATTGCTGACCGGTGC
TGCNAGTTGTACCTGCCCGGGCGGCCGCTCTAGAACTA
Sequence 4715
GCCGCCGGGCAGGTACAAACTTAGAAGAAAATNGGAAGATAGAAACAAGATAGAAAATGA
AAATATTGTCAAGAGTTTCAGATAGAAAATGAAAAACAAGCTAAGACAAGTATTGGAGAA
GTATAGAAGATAGAAAAATATAAAGCCAAAAATTGGATAAAATAGCACTGAAAAAATGAG
GAAATTATTGGTAACCAATTTATTTTAAAAGGCCATCAATTTAATTTCTGGTGGTGCAGA
AGTTAGAAGGTAAAGCTTGAGAAGATGAGGGTGTTTACGGTAGACCNGAACCAATTTAGA
AGAATACTTGAAGCTAGAAGGGGAAGTTGGTTAAAAATCACATCAAAAAGCTACTAAAAG
GACTGGTGTAATTTTAAAAAAAACTAAGGCAGAAGGCTTTTGGAAGAAGTTAGAAGAATT
TGGAAGGCCTTAAATATAGTAGCTTAATTTGAAAAAATGTGAAGGACCTTTCGTAACCGG
AAGGTNATTTCAAGATNAAAGAAGTAATTTACCAACCTTAATGGTTTTTGCCTTTGGGGC
TTTTGAGTTTAAAGAATTANT
Sequence 4716
GAATNGGAGCTCCCCGCGGTGGCGGCCGANGNACTCNGGANAGAGTANAAACGTTTCCAT
GTTATTTCTGCAGTTGTNGACTTAGGCTTATTTGTAAAGAAGCATGCTCCATTGACTGCC
ATCTCTAGTCTTGCAGTGGGTGGTATTAACCCATAGAAAGCAAGCAGTTGTGTATCACAT
ACACAATGGNTATGATGNTAACCANATCANCTGTTTNGTTGTTCATTCGTNATATGTTTT
GTGATANGGATGTTGGGAGCACAGCTCTATTCTGCCTGCTCAGACTTAAGTTAGACCCTT
ATCTTTTATATTATGTCATGAAAAAAGTCTCCTAAAAATTGTGAAACTAGCCTCTTGATG
AGTGATG
Sequence 4717
CCGCGGTGGCGGCCCGAGGTACATGGCCACAAGAGGAGCCCTGGCCAACAGTTTCAGGCT
GGCCATCTTGGGCTGGGGCTCCCTGGGCTTGCAGTGGGGAGGGTCAGGATTCAAATTCTG
GGTGACAATTTCAGTAGCACCAAGACCAGAAGACAGTGAGGCCACGCTGGGGAGTGGGGG
AGTGCTTGTATGTGGGATGCATGGAGGGAGGCTGCAGTATGTGGAGGAGAAAGAGAGGCT
GTTGTGGGCTGGTTCTTAAGACACTCAAGGGGACTCAAAGGGTCGGCTCCAGCCATGGAC
ACTATCTAAGGCTGCTCAGAGGAGAATGCTGGAGGAGAGGAAGAGGAAGTGAAATGGTGT
GAGAACATTCTTACCCCTATAAATAAACTATTTACACACTTTAAGAAAAGGAGAGCCGGC
TCTCAGTCCCGCGTACCTGCCCGGCCGGGCCCGCTCTAGAACTA
Sequence 4718
CCGCGGTGGCGGCCGAGGTACGCGGGGACCTCAGCCCAGCAATTCGTTTGGAGCATGTGA
ACACCTTGAGCCTTGATGAGTTCCAGTATGTGGTATATTATGCAGAGCATTCAGAGCAAA
TACTCTCTCTCCGAGCGCTTAATCCGAACAATTGCTGCCATCCGTTCCTTCCCACATGAT
AATGTAGAGGACCTCATCAGAGGGGGAGCAGATGTGAACTGCACTCATGGCACACTGAAG
CCCTTGCACTGTGCCTGTATGGTGTCAGATGCTGACTGTGTGGAGTTACTTCTGGAAAAA
GGAGCCGAGGTGAATGCCCTGGATGGGTATAACCCGAACAGCCCTCCACTATGCANCAGA
GAAAGATGAGGCTTGTGTGGAGGTCCTATTGGAGTATGGTGCAAACCCCAATGCTTTGGA
TGGCAACAGAGATACCCCACTTCACTGGGTAGCCTTTAAGAACAATGCTGAGTGTGTGCG
GGCTCTCCTAGAGAGCGGGGCCTCTGTCAATGCCCTGGA
Sequence 4719
AAATCCGGCGAATNGTTTCTNCACCGCGGTGGCGGNCGCCCGGGCAGGTACNCGGGTATG
CTGCCTGGAGACACGCAGATTTNCTGTCTANTCACACACACACGCAGACATGCTGTCCGG
ACACACACACGCATGCACAGATATGCTGTCCGGACACACACACGCACGCANATATGCTTG
CCTGGACACACACACAGATAATGCTGCCTCAACACTCACACACGTGCAGATATTGCCTGG
ACACACACATGTGCACAGATATGCTGTCTGGACATGCACACACCGTGCAGATATGCTGTC
CGGATACACACGCACGCACACATGCAGATATGCTGCCTGGGCACACACTTTCGGACACAC
ATGCACACACAGGTGCAGATATTGCTGCCTGGACACACGCAGACTGNACGTGCTTTTGGG
AG
Sequence 4720
AGGAGTGCAGAACCCTGTANCCCGCTGCATCATGTGCTGTTTCAAGNGCTGCCTCTGGTG
TCTGGAAAAATTTATCAAGTTCCTAAACCNCAATGCATACATCATGATCGCCATCTNCGG
GAAGAATTTNTGTGTCTCAGCCAAAAACGCGTTCATGCTNCTCATGCGAAACATTGTCAG
GGNGGTCGTCCTGGACAAAGTCACAGACCTGCTGNTGTTCTTTGGGAAGCTGCTGGTGGT
CGGAGGCGTGGGGGTCCTGTCCTTCTTTTTTTTCTCCGGTCGCATNCCGGGGCTGGGATA
AAGACTTTAANAGCCCCCACCTNAACTATTACTGGCTGCCCCATCATGACCCTNCANTCC
Sequence 4721
CGCCCGGGCAGGTACATTCTCATCATTAGGATTGGTTGTTGCTGTCCTTGCACAACTGGT
TAAGGAAAAATGAATTATTCTGTAATTTCTGAAGAATCCAAATCCTGTCTCTTATAAAGT
CAGAACAGAAGGGGGCAAAAGGTGGTGGTGAGCATCAAGAGAGAAAAAAGGAGAAAATTA
TTTACAGAAAATAGGAGACANGAGGGAGTGTCCGCAAGAAAAGACTTCATTGTCACTTCT
TCTTGCCGGCCCTCTTGGCACTGGACTTTGCTTTGGGTTTCACTGGTTTGGCCTTTTTGG
GCTTGGATGCCTTTGACCGGCTTGGCTTTGACAGTCTTGGGTTTTTTTGGCNTTCTTGGG
CGTGGCAGCCAAGCTTCTTCTTGGCCTTCTTGACCCGGGGTGGCTTTNGGTTTTCTTTGG
NTTGGGGG
Sequence 4722
GGTGGCGGCCGAGGTACCTCAAAAGTGGGGAAATCCCCATATAACTGANGACGAAGGCAG
TTCAGAAGTTCATTGATTTGCCCTAAGGTTCCTCAATTTGCAAACGTCAGGCCAATGATC
CAACCCCAGGTATGTTTGGCAGTGAAGGACCAGTTGAGTCATAGCTGCAAGTAACCACCC
TGCAGTGGTCCCTATCTTGGCCGTTAGCTTACATTGACATTTAACACTCAAATTTACTCA
GTAACACCAGCTATCATGTTTTCCACTAAAACTCCACAGCATTCTGGCAACTTTTCTATT
TTAGAGCAATAAAGTAAATTGTTAGCATCCCTTTGACATATAAATATTTCTACAAATAGT
AATTCTCTAGCCATTCATTTGGAGTATTTAAAACTCAACATTCATAGCACATTTTATGGT
GACAAAGAACTTATGTTCAGAACACAAAAAATAAGTCGTACCTGCCCGGGCGGGCGGTCT
TNGAACTAGTG
Sequence 4723
ACAAAACCAATCTACCTGATGAAAACTCCGTTCCCTTCTCGCCAGAAACATAAAATGCG
ATGGAGCTACGGCCACCGCTGCCGAGACAAAATGGCGCCGACCCCCGCGTACCT
Sequence 6889
GAGCTCNCCGCGGTGGCGGCCGCCCGGGCAGGTACTTTTTTTTTTTCTCCGGTCGCATCC
CGGGGCTGGGTAAAGACTTTAAGAGCCCCCACCTCAACTATTACTGGCTGCCCATCATGA
CCTCCATCCTGGGGGCCTATGTCATCGCCAGCGGCTTTTTTCAGCGGTTTTCGGCATGTG
TGTGGACACGCCCTTCCTCTGCTTCCTGGAAGACCTGGAGCGGAACAACGGCTCCCTGGA
CCGGCCCTACTACATGTCCAAGAGCCTTCTAAAGATTCTGGGCAAGAAGAACGAGGCGCC
CCCGGACAACAAGAAGAGGAAGGAAGTGACAGCTCCGGCCCTGATCCAGGACTGCACCCC
ACCCCCACCGTCCAGCCATTCAACCTTACTTCGCCTTACAGGGTC
Sequence 6890
CTCACTATAGGGCGAATTGGGAGCTCCCCGCGGTGGCGGCCCGCCCGGGCAGGTACTATA
TGGACACCAACATGGAGCAGAAAATGAGGGCAGCAGTGTCCAATCTCATTCCAGGTGAGA
AGTTGCACAGTGTCCAATAGGTGCATACATCTCCTTAGTAAGTAGTTGTGATTAACAATG
AAATAGAAATGAAAAATATATTTTTTTATTTATGTGTATTATATTTTTCAAGCAGCTAAT
AAGTTGGTAGGACATAATATTTAATTCGTTGGGGACCTAATTATTTATAAATTGAATGGT
TAGATATTTCTTTTGGCCTAAGCCACCATGAAAAAATCACTGAGGCGCTAAGGGAAACAT
GAACTAAGAAGCCCTTCTGAGTCTCTGTTTTCTCAACTGTAAGATGAAGAAACCTACTCC
ACCTTCCTCTTGCGATTGTTGAACGGCAGTTGAGATACTGCGCAGAATGGGACCTTATTG
ATGGCCTACCCAACATCCATTCTCTACTCCCTCTACTCTGATGGCACCCG
Sequence 6891
CCGCGGTGGCGGCCGAGGTACGCGGGGTTTTCAGGGTCGTAGGACGCCGTTGGGCACCAC
GCTCGGAGAAGGACAGGACAATGGCGGCCTTAGGGTCCCCGTCGCACACTTTTCGAGGAC
TTCTGCGGGAGTTGCGCTACCTGAGCGCGGCCACCGGCCGCTCTAGAACTAAGTGGATCC
CCCCGGCTTGCANGA
Sequence 6892
CTCCCCGCGGTGGCGGCCGAGGTACGCGGGGCTCTATATAAGTGGGCAGNGGCCGNGACT
GCGCGCAGACACTGACCTTCAGCGCCTNGGCTCCAGCGCCATGGCGCCCTCCAGGAAGTT
CTTCGTTGGGGGAAACTGGAAGATGAACGGACGGAAGCAGANTGTGGGGGAGCTCATCGG
CACTCTNAACGCGGCCAA
Sequence 6893
CCACCGCGGTGGCGGCCGAGGTACAGTGGCACCTGGGAAAAGGCACCTGGAAGGTTTCCA
TGTGGCCCAGCCCAGCATGGAAGCAGGGTGGGAACTCTGCTGTGTCGCCAGCGCTCACTC
TACTCGAGTGGCTTTTTGAAAGCCCTACCATGTCTGTGTCAGGCCTGTGCTGCTTCACAT
CCTACAGCTGCCTAGGAAAGGCCGGCCACGCTCCCTGTCCACACACTCCCTGTCCACACA
CTCCCTGTCCACACACTCCCTGTCCACAACTGCAGCCGGGCCCTCTGCCTATGGGCACCC
AATCCAAGCAGCTGCTCCACCTTTGTTTGGCATGGTGATTTGTGTTTTTTCTCTTGGCGC
TTATGTGTGTGGGCTTGGGACGAAGTGCTGGTATGCACTTAGGACCTTCTTGATAGCTCC
CTGCACTTTGGAACACGGAGCAAATGAAAAAAGGGTCGGGGCTTGCCCTCCACCTTGGAC
TTGGAAGAAGCCCACATTGGAGANGTGAAGGACCCCATGGTGGCTCTAGTGGGAAAATAC
GTTAGCCTCAANCTNAGGANGGATGAGGCCAACCCCAANAGGGAGACCTTAATTGATAGG
GGATCANGCTAAAAAAATGG
Sequence 6894
AGGTACTCGCGCTGTTCTTCTGTTATCATCCAGGGTTCGTCGGGGTAACTGCTGTTATCC
TGTAGTTCCCTCTCCACTGAAAAAGACCGGATAAGGGAAGCCTGATGCCGAAGGGGCTTG
GTTCCTGGGCCCCCTGATGAGGATCCCTCGGGCTGGACAAGGGGCTGCCTAGCTTCATAA
GGTGCTGGATGCATCTGTTCTGCGTTGCTCCGCAGTTTGCTGTAGCCATGGCTCAAGGGC
ACCCTCTGGTAATGAGGCGGGGAAGAAGGAGGGGAGGCGAGGGGTGACATCGTGGGAGAC
TGTGTTTCCTGCTGTTTATCCTCATCGTCCATTCTTTTGAAGGAATTTT
Sequence 6895
CCGCGGTGGCGGCCGCCCGGGCAGGTACGCGGGGATTGTGGCAGCTGGAGGTCTCTGCAT
CGCTTATCCGTTTATTAGCCGGACCAAGATTGCACAGCTAAAGTCTGGCAGAGACTCCAC
GGTATGACTGTCCTCACTGGGCCTGTCCACAGTGCGAGCGACTCCTGAGGGGAACAGCGC
CGGAGTTCAGGAGTCCAAGCACAAAGCGGTCTTTTACATTCCAACCTGTTGCCTGCCAGC
CCTTTCTGGATTACTGATAGAAAATCATGCAAAACCTCCCAACCTTTCTAAGGACAAGAC
TACTGTGGATTCAAGTGCTTTAATGACTATTTATGCGTTGACTGTGAGAATAGGGAGCAG
NGCCATGGGACATTTCTAGGTGTAGAGAAAGAAGAAACTGCAATGGAAAAATTTGTATGA
TTTCCATTTATTTC
Sequence 6896
CCGCGGTGGCGGCCGAGGTACAGTGGTTAATTTGTCACAACTGACTGAGGCATCACAACA
ACAGCAGCAGTCACCACTACAAGAACAAGCACAGACTTTACAGCAGCAGATTTCATCAAA
TATTTTTCCATCACCAAATAGTAACAACATGCCTGGAATTCAAGGAGCCACATCTTCGCC
TTCAACCACAGGCTACTTTTATTTCACAACACAGCAGGAGGCACAATGAACCAACTGCAG
AATTCTCCTGGCTCATCTCAGCAGACATCAGGAATGTTCTTATTTGGCATTCAAAATAAC
TGTAGTCAGCTTTTAACCTCTGGACCAGCTACATTGCCTGATCAGTTGATGGCCATAAGT
CAGCCAGGCCAACCACAAAACGAGGGCCAGCCACCTGTGACAACACTTCTTTCTCAGCAA
ATGCCAGAGAATTCTCCACTGGCATCCTCTATAAACACCAACCAGAACATCGAAAAGATT
Sequence 6897
CTACTTAGGGCGAATTGGAGCTCCCCGCGGTGGCGGCCGCCCGGGCAGGTACTCTGGTCA
TCTTCGTTCGTTTGGTCGTGCAAGGTGTTAACTATTTTCACTTCCCATATCACAAAGTTA
GTCCACAGGAGGAGCTGGTGGATCTTGTCCATTATGAGGACTGGTTGGCTTTCCAGGTAG
GCTGGATCCTNTTAGATTAGGAGGGTCTCAGTAAGAACAGGATCAATGCAATAACCATCC
AGGCTACCAAGATNATTGTAACACTGATGCCNTTATNACCAGAGGGTCCCGGTAATTNCT
GAANACACTCTGTGTCTGTGCAGTATGATTGGGACTGCCGTANCANATTGNTCAGTCTTC
TCANTGCATNGTTTCATGAGAGCAAAACACATTNACAGGGATCAAATTCCACCTTCTTGC
CCATTATTACCCAGCTTGGATTTTTACTTGGACCTNTTTTTAAAATCCCNCC

TABLE 3-3

| Sequence # | Acc No | Database hit | GI Number |
|---|---|---|---|
| Sequence 7512 | A14133 | Genbank | 490127 |
| Sequence 7513 | A26481 | Genbank | 1247452 |
| Sequence 7514 | A26595 | Genbank | 1247460 |
| Sequence 7515 | A29216 | Genbank | 1247528 |
| Sequence 7516 | A33292 | Genbank | 1567852 |
| Sequence 7517 | A36460 | Genbank | 2293778 |
| Sequence 7518 | A43521 | Genbank | 2298710 |
| Sequence 7519 | A58331 | Genbank | 3713993 |
| Sequence 7520 | A80239 | Genbank | 6092971 |
| Sequence 7521 | A81324 | Genbank | 6731650 |
| Sequence 7522 | A98295 | Genbank | 6781400 |
| Sequence 7523 | AB000360 | Genbank | 2547041 |
| Sequence 7524 | AB000468 | Genbank | 1843400 |
| Sequence 7525 | AB000516 | Genbank | 2723379 |
| Sequence 7526 | AB000584 | Genbank | 1813326 |
| Sequence 7527 | AB000624 | Genbank | 5748486 |
| Sequence 7528 | AB000889 | Genbank | 2467299 |
| Sequence 7529 | AB001523 | Genbank | 2244605 |
| Sequence 7530 | AB001601 | Genbank | 3107912 |
| Sequence 7531 | AB002292 | Genbank | 2224528 |
| Sequence 7532 | AB002293 | Genbank | 2224530 |
| Sequence 7533 | AB002299 | Genbank | 2224542 |
| Sequence 7534 | AB002310 | Genbank | 2224564 |
| Sequence 7535 | AB002313 | Genbank | 2280475 |
| Sequence 7536 | AB002323 | Genbank | 2224590 |
| Sequence 7537 | AB002328 | Genbank | 2224600 |
| Sequence 7538 | AB002336 | Genbank | 2224616 |
| Sequence 7539 | AB002346 | Genbank | 6634018 |
| Sequence 7540 | AB002351 | Genbank | 2224646 |
| Sequence 7541 | AB002352 | Genbank | 2224648 |
| Sequence 7542 | AB002356 | Genbank | 2224656 |
| Sequence 7543 | AB002365 | Genbank | 2224674 |
| Sequence 7544 | AB002368 | Genbank | 2224680 |
| Sequence 7545 | AB002374 | Genbank | 2280484 |
| Sequence 7546 | AB002375 | Genbank | 2280486 |
| Sequence 7547 | AB002377 | Genbank | 6634024 |
| Sequence 7548 | AB002381 | Genbank | 2224706 |
| Sequence 7549 | AB002382 | Genbank | 2224708 |
| Sequence 7550 | AB002387 | Genbank | 2224718 |
| Sequence 7551 | AB002559 | Genbank | 1944129 |
| Sequence 7552 | AB003476 | Genbank | 2081606 |
| Sequence 7553 | AB003730 | Genbank | 2627128 |
| Sequence 7554 | AB005754 | Genbank | 6456482 |
| Sequence 7555 | AB006630 | Genbank | 2564331 |
| Sequence 7556 | AB006651 | Genbank | 3201570 |
| Sequence 7557 | AB007447 | Genbank | 2463530 |
| Sequence 7558 | AB007510 | Genbank | 2463576 |
| Sequence 7559 | AB007859 | Genbank | 6634028 |
| Sequence 7560 | AB007882 | Genbank | 2887418 |
| Sequence 7561 | AB007895 | Genbank | 2662150 |
| Sequence 7562 | AB007899 | Genbank | 2662158 |
| Sequence 7563 | AB007900 | Genbank | 2662160 |
| Sequence 7564 | AB007920 | Genbank | 3413863 |
| Sequence 7565 | AB007930 | Genbank | 3413883 |
| Sequence 7566 | AB007931 | Genbank | 3413885 |
| Sequence 7567 | AB007944 | Genbank | 3413911 |
| Sequence 7568 | AB007957 | Genbank | 3413931 |
| Sequence 7569 | AB007965 | Genbank | 3413940 |
| Sequence 7570 | AB009398 | Genbank | 3618342 |
| Sequence 7571 | AB011086 | Genbank | 3043551 |
| Sequence 7572 | AB011087 | Genbank | 3043553 |
| Sequence 7573 | AB011096 | Genbank | 3043571 |
| Sequence 7574 | AB011113 | Genbank | 3043605 |
| Sequence 7575 | AB011144 | Genbank | 3043667 |
| Sequence 7576 | AB011149 | Genbank | 3043677 |
| Sequence 7577 | AB011151 | Genbank | 3043681 |
| Sequence 7578 | AB011155 | Genbank | 3043689 |
| Sequence 7579 | AB011166 | Genbank | 3043711 |
| Sequence 7580 | AB011167 | Genbank | 3043713 |
| Sequence 7581 | AB011169 | Genbank | 3043717 |
| Sequence 7582 | AB011175 | Genbank | 3043729 |
| Sequence 7583 | AB011536 | Genbank | 3449297 |
| Sequence 7584 | AB011542 | Genbank | 3449309 |
| Sequence 7585 | AB012124 | Genbank | 4126408 |
| Sequence 7586 | AB012193 | Genbank | 3599675 |
| Sequence 7587 | AB012911 | Genbank | 3062802 |
| Sequence 7588 | AB012922 | Genbank | 4126426 |
| Sequence 7589 | AB014087 | Genbank | 5672599 |
| Sequence 7590 | AB014535 | Genbank | 3327083 |
| Sequence 7591 | AB014540 | Genbank | 3327093 |
| Sequence 7592 | AB014542 | Genbank | 3327097 |
| Sequence 7593 | AB014557 | Genbank | 3327127 |
| Sequence 7594 | AB014563 | Genbank | 3327139 |
| Sequence 7595 | AB014566 | Genbank | 3327145 |
| Sequence 7596 | AB014569 | Genbank | 3327151 |
| Sequence 7597 | AB014589 | Genbank | 3327191 |
| Sequence 7598 | AB014592 | Genbank | 3327197 |
| Sequence 7599 | AB014597 | Genbank | 3327207 |
| Sequence 7600 | AB015597 | Genbank | 4115713 |
| Sequence 7601 | AB017114 | Genbank | 4586879 |
| Sequence 7602 | AB017335 | Genbank | 3582440 |
| Sequence 7603 | AB017498 | Genbank | 3582144 |
| Sequence 7604 | AB017548 | Genbank | 3885363 |
| Sequence 7605 | AB018247 | Genbank | 4049592 |
| Sequence 7606 | AB018255 | Genbank | 3882144 |
| Sequence 7607 | AB018281 | Genbank | 3882196 |
| Sequence 7608 | AB018290 | Genbank | 3882214 |
| Sequence 7609 | AB018302 | Genbank | 3882238 |
| Sequence 7610 | AB018315 | Genbank | 3882264 |
| Sequence 7611 | AB018316 | Genbank | 3882266 |
| Sequence 7612 | AB018324 | Genbank | 3882282 |
| Sequence 7613 | AB018331 | Genbank | 3882296 |
| Sequence 7614 | AB018335 | Genbank | 3882304 |
| Sequence 7615 | AB018342 | Genbank | 3882318 |
| Sequence 7616 | AB018347 | Genbank | 3882328 |
| Sequence 7617 | AB018350 | Genbank | 3882334 |
| Sequence 7618 | AB018353 | Genbank | 3882340 |
| Sequence 7619 | AB018357 | Genbank | 6009486 |
| Sequence 7620 | AB019564 | Genbank | 3885367 |
| Sequence 7621 | AB019568 | Genbank | 3885371 |
| Sequence 7622 | AB020315 | Genbank | 3925828 |
| Sequence 7623 | AB020335 | Genbank | 6518494 |
| Sequence 7624 | AB020623 | Genbank | 3985929 |
| Sequence 7625 | AB020631 | Genbank | 4240136 |
| Sequence 7626 | AB020645 | Genbank | 4240164 |
| Sequence 7627 | AB020659 | Genbank | 4240192 |
| Sequence 7628 | AB020662 | Genbank | 4240198 |
| Sequence 7629 | AB020666 | Genbank | 6635134 |
| Sequence 7630 | AB020674 | Genbank | 4240222 |
| Sequence 7631 | AB020679 | Genbank | 4240232 |
| Sequence 7632 | AB020692 | Genbank | 4240258 |
| Sequence 7633 | AB020697 | Genbank | 4240268 |
| Sequence 7634 | AB020698 | Genbank | 4240270 |
| Sequence 7635 | AB020713 | Genbank | 4240300 |
| Sequence 7636 | AB020714 | Genbank | 4240302 |
| Sequence 7637 | AB020718 | Genbank | 4240310 |
| Sequence 7638 | AB020721 | Genbank | 4240316 |
| Sequence 7639 | AB020764 | Genbank | 6467144 |
| Sequence 7640 | AB020874 | Genbank | 4003394 |
| Sequence 7641 | AB021663 | Genbank | 4996450 |
| Sequence 7642 | AB022537 | Genbank | 6942217 |
| Sequence 7643 | AB023049 | Genbank | 5672604 |
| Sequence 7644 | AB023050 | Genbank | 5672605 |
| Sequence 7645 | AB023051 | Genbank | 5672606 |
| Sequence 7646 | AB023052 | Genbank | 5672607 |
| Sequence 7647 | AB023137 | Genbank | 4589471 |
| Sequence 7648 | AB023145 | Genbank | 4589487 |
| Sequence 7649 | AB023150 | Genbank | 4589509 |
| Sequence 7650 | AB023152 | Genbank | 4589513 |
| Sequence 7651 | AB023160 | Genbank | 4589529 |
| Sequence 7652 | AB023165 | Genbank | 4589539 |
| Sequence 7653 | AB023195 | Genbank | 4589599 |
| Sequence 7654 | AB023199 | Genbank | 4589607 |
| Sequence 7655 | AB023205 | Genbank | 4589619 |
| Sequence 7656 | AB023420 | Genbank | 4579908 |
| Sequence 7657 | AB024704 | Genbank | 4589928 |
| Sequence 7658 | AB025580 | Genbank | 5931568 |
| Sequence 7659 | AB026906 | Genbank | 5295993 |
| Sequence 7660 | AB028893 | Genbank | 6552364 |
| Sequence 7661 | AB028942 | Genbank | 5689368 |
| Sequence 7662 | AB028952 | Genbank | 5689394 |
| Sequence 7663 | AB028985 | Genbank | 5689460 |
| Sequence 7664 | AB028986 | Genbank | 5689462 |
| Sequence 7665 | AB028990 | Genbank | 5689470 |
| Sequence 7666 | AB028999 | Genbank | 5689488 |
| Sequence 7667 | AB029002 | Genbank | 5689494 |
| Sequence 7668 | AB029003 | Genbank | 5689496 |
| Sequence 7669 | AB029008 | Genbank | 5689506 |
| Sequence 7670 | AB029013 | Genbank | 5689516 |
| Sequence 7671 | AB029017 | Genbank | 5689524 |
| Sequence 7672 | AB029019 | Genbank | 5689528 |
| Sequence 7673 | AB029025 | Genbank | 5689540 |
| Sequence 7674 | AB029026 | Genbank | 5689542 |
| Sequence 7675 | AB029027 | Genbank | 5689544 |
| Sequence 7676 | AB031051 | Genbank | 6683742 |
| Sequence 7677 | AB032417 | Genbank | 6277265 |
| Sequence 7678 | AB032963 | Genbank | 6329896 |
| Sequence 7679 | AB032975 | Genbank | 6330044 |
| Sequence 7680 | AB032978 | Genbank | 6330094 |
| Sequence 7681 | AB032983 | Genbank | 6330128 |
| Sequence 7682 | AB033007 | Genbank | 6330242 |
| Sequence 7683 | AB033011 | Genbank | 6330301 |
| Sequence 7684 | AB033018 | Genbank | 6330350 |
| Sequence 7685 | AB033023 | Genbank | 6330384 |
| Sequence 7686 | AB033026 | Genbank | 6330406 |
| Sequence 7687 | AB033027 | Genbank | 6330415 |
| Sequence 7688 | AB033033 | Genbank | 6330496 |
| Sequence 7689 | AB033051 | Genbank | 6330667 |
| Sequence 7690 | AB033058 | Genbank | 6330721 |
| Sequence 7691 | AB033070 | Genbank | 6330818 |
| Sequence 7692 | AB033073 | Genbank | 6330839 |
| Sequence 7693 | AB033078 | Genbank | 6330873 |
| Sequence 7694 | AB033104 | Genbank | 6331314 |
| Sequence 7695 | AB034205 | Genbank | 6899845 |
| Sequence 7696 | AB035519 | Genbank | 6681348 |
| Sequence 7697 | AB035698 | Genbank | 6970477 |
| Sequence 7698 | AB037728 | Genbank | 7242968 |
| Sequence 7699 | AB037743 | Genbank | 7243024 |
| Sequence 7700 | AB037748 | Genbank | 7243034 |
| Sequence 7701 | AB037761 | Genbank | 7243060 |
| Sequence 7702 | AB037763 | Genbank | 7243064 |
| Sequence 7703 | AB037775 | Genbank | 7243088 |
| Sequence 7704 | AB037783 | Genbank | 7243104 |
| Sequence 7705 | AB037784 | Genbank | 7243106 |
| Sequence 7706 | AB037797 | Genbank | 7243132 |
| Sequence 7707 | AB037801 | Genbank | 7243140 |
| Sequence 7708 | AB037807 | Genbank | 7243152 |
| Sequence 7709 | AB037819 | Genbank | 7243176 |
| Sequence 7710 | AB037844 | Genbank | 7243226 |
| Sequence 7711 | AB037847 | Genbank | 7243232 |
| Sequence 7712 | AB037856 | Genbank | 7243267 |
| Sequence 7713 | AC000024 | Genbank | 2995601 |
| Sequence 7714 | AC000031 | Genbank | 4646246 |
| Sequence 7715 | AC000036 | Genbank | 7958979 |
| Sequence 7716 | AC000049 | Genbank | 5705995 |
| Sequence 7717 | AC000083 | Genbank | 3810636 |
| Sequence 7718 | AC000087 | Genbank | 7124755 |
| Sequence 7719 | AC000088 | Genbank | 7124834 |
| Sequence 7720 | AC000095 | Genbank | 7940365 |
| Sequence 7721 | AC000114 | Genbank | 1809233 |
| Sequence 7722 | AC000134 | Genbank | 4755212 |
| Sequence 7723 | AC000353 | Genbank | 6970735 |
| Sequence 7724 | AC000378 | Genbank | 2270906 |
| Sequence 7725 | AC002064 | Genbank | 2076723 |
| Sequence 7726 | AC002066 | Genbank | 2076721 |
| Sequence 7727 | AC002094 | Genbank | 2155224 |
| Sequence 7728 | AC002105 | Genbank | 2288973 |
| Sequence 7729 | AC002117 | Genbank | 2281075 |
| Sequence 7730 | AC002347 | Genbank | 2828783 |
| Sequence 7731 | AC002377 | Genbank | 2275192 |
| Sequence 7732 | AC002401 | Genbank | 2599240 |
| Sequence 7733 | AC002470 | Genbank | 6478944 |
| Sequence 7734 | AC002522 | Genbank | 7124014 |
| Sequence 7735 | AC002550 | Genbank | 2570261 |
| Sequence 7736 | AC002553 | Genbank | 3126783 |
| Sequence 7737 | AC003010 | Genbank | 2981254 |
| Sequence 7738 | AC003025 | Genbank | 3337308 |
| Sequence 7739 | AC003031 | Genbank | 2570227 |
| Sequence 7740 | AC003036 | Genbank | 2935595 |
| Sequence 7741 | AC003098 | Genbank | 2822155 |
| Sequence 7742 | AC003103 | Genbank | 2842782 |
| Sequence 7743 | AC003663 | Genbank | 3097871 |
| Sequence 7744 | AC003665 | Genbank | 3808091 |
| Sequence 7745 | AC003687 | Genbank | 2828778 |
| Sequence 7746 | AC003950 | Genbank | 4262275 |
| Sequence 7747 | AC003963 | Genbank | 2995604 |
| Sequence 7748 | AC004016 | Genbank | 2781374 |
| Sequence 7749 | AC004017 | Genbank | 2795792 |
| Sequence 7750 | AC004020 | Genbank | 3219330 |
| Sequence 7751 | AC004080 | Genbank | 2822164 |
| Sequence 7752 | AC004084 | Genbank | 2822156 |
| Sequence 7753 | AC004098 | Genbank | 3097872 |
| Sequence 7754 | AC004126 | Genbank | 3242744 |
| Sequence 7755 | AC004150 | Genbank | 4585948 |
| Sequence 7756 | AC004228 | Genbank | 4263838 |
| Sequence 7757 | AC004234 | Genbank | 2914672 |
| Sequence 7758 | AC004242 | Genbank | 3132845 |
| Sequence 7759 | AC004253 | Genbank | 3169206 |
| Sequence 7760 | AC004263 | Genbank | 2935616 |
| Sequence 7761 | AC004382 | Genbank | 3252819 |
| Sequence 7762 | AC004410 | Genbank | 2959558 |
| Sequence 7763 | AC004472 | Genbank | 2984582 |
| Sequence 7764 | AC004475 | Genbank | 2988396 |
| Sequence 7765 | AC004500 | Genbank | 2996639 |
| Sequence 7766 | AC004522 | Genbank | 3006227 |
| Sequence 7767 | AC004526 | Genbank | 3779035 |
| Sequence 7768 | AC004551 | Genbank | 3309005 |
| Sequence 7769 | AC004583 | Genbank | 3293210 |
| Sequence 7770 | AC004696 | Genbank | 3135391 |
| Sequence 7771 | AC004703 | Genbank | 3228509 |
| Sequence 7772 | AC004707 | Genbank | 3249127 |
| Sequence 7773 | AC004757 | Genbank | 3341707 |
| Sequence 7774 | AC004770 | Genbank | 3212836 |
| Sequence 7775 | AC004771 | Genbank | 3668108 |
| Sequence 7776 | AC004801 | Genbank | 4204244 |
| Sequence 7777 | AC004812 | Genbank | 3970970 |
| Sequence 7778 | AC004821 | Genbank | 4753291 |
| Sequence 7779 | AC004823 | Genbank | 4156207 |
| Sequence 7780 | AC004835 | Genbank | 4508154 |
| Sequence 7781 | AC004854 | Genbank | 4827328 |
| Sequence 7782 | AC004857 | Genbank | 3694665 |
| Sequence 7783 | AC004882 | Genbank | 5523837 |
| Sequence 7784 | AC004893 | Genbank | 3694662 |
| Sequence 7785 | AC004938 | Genbank | 3213059 |
| Sequence 7786 | AC004966 | Genbank | 5306307 |
| Sequence 7787 | AC004997 | Genbank | 5441941 |
| Sequence 7788 | AC005032 | Genbank | 5649384 |
| Sequence 7789 | AC005037 | Genbank | 4827310 |
| Sequence 7790 | AC005041 | Genbank | 4508118 |
| Sequence 7791 | AC005042 | Genbank | 4156138 |
| Sequence 7792 | AC005058 | Genbank | 4156135 |
| Sequence 7793 | AC005071 | Genbank | 4508112 |
| Sequence 7794 | AC005081 | Genbank | 6560914 |
| Sequence 7795 | AC005088 | Genbank | 4753221 |
| Sequence 7796 | AC005104 | Genbank | 4218027 |
| Sequence 7797 | AC005192 | Genbank | 3264575 |
| Sequence 7798 | AC005214 | Genbank | 3282166 |
| Sequence 7799 | AC005225 | Genbank | 6094661 |
| Sequence 7800 | AC005253 | Genbank | 3288886 |
| Sequence 7801 | AC005318 | Genbank | 3885345 |
| Sequence 7802 | AC005324 | Genbank | 3366582 |
| Sequence 7803 | AC005325 | Genbank | 3366581 |
| Sequence 7804 | AC005339 | Genbank | 3355454 |
| Sequence 7805 | AC005366 | Genbank | 3367508 |
| Sequence 7806 | AC005369 | Genbank | 3367505 |
| Sequence 7807 | AC005392 | Genbank | 3399669 |
| Sequence 7808 | AC005480 | Genbank | 4835818 |
| Sequence 7809 | AC005488 | Genbank | 5836194 |
| Sequence 7810 | AC005516 | Genbank | 3900835 |
| Sequence 7811 | AC005519 | Genbank | 5091653 |
| Sequence 7812 | AC005523 | Genbank | 3451032 |
| Sequence 7813 | AC005531 | Genbank | 4153875 |
| Sequence 7814 | AC005538 | Genbank | 4508129 |
| Sequence 7815 | AC005546 | Genbank | 3478635 |
| Sequence 7816 | AC005549 | Genbank | 3598724 |
| Sequence 7817 | AC005612 | Genbank | 3540153 |
| Sequence 7818 | AC005629 | Genbank | 7243877 |
| Sequence 7819 | AC005670 | Genbank | 3688099 |
| Sequence 7820 | AC005686 | Genbank | 3608161 |
| Sequence 7821 | AC005726 | Genbank | 3810672 |
| Sequence 7822 | AC005754 | Genbank | 3688074 |
| Sequence 7823 | AC005768 | Genbank | 6598827 |
| Sequence 7824 | AC005789 | Genbank | 3702281 |
| Sequence 7825 | AC005828 | Genbank | 3789713 |
| Sequence 7826 | AC005841 | Genbank | 4731044 |
| Sequence 7827 | AC005899 | Genbank | 3935221 |
| Sequence 7828 | AC005921 | Genbank | 4982556 |
| Sequence 7829 | AC005923 | Genbank | 4309927 |
| Sequence 7830 | AC005924 | Genbank | 4309926 |
| Sequence 7831 | AC005988 | Genbank | 4156128 |
| Sequence 7832 | AC006026 | Genbank | 6042101 |
| Sequence 7833 | AC006040 | Genbank | 4314426 |
| Sequence 7834 | AC006042 | Genbank | 4508120 |
| Sequence 7835 | AC006064 | Genbank | 4572650 |
| Sequence 7836 | AC006077 | Genbank | 3935193 |
| Sequence 7837 | AC006088 | Genbank | 4204701 |
| Sequence 7838 | AC006146 | Genbank | 5836157 |
| Sequence 7839 | AC006165 | Genbank | 3980464 |
| Sequence 7840 | AC006241 | Genbank | 4160141 |
| Sequence 7841 | AC006252 | Genbank | 4309923 |
| Sequence 7842 | AC006255 | Genbank | 5306223 |
| Sequence 7843 | AC006299 | Genbank | 4106997 |
| Sequence 7844 | AC006441 | Genbank | 4760435 |
| Sequence 7845 | AC006449 | Genbank | 6102666 |
| Sequence 7846 | AC006478 | Genbank | 4508145 |
| Sequence 7847 | AC006480 | Genbank | 6289252 |
| Sequence 7848 | AC006487 | Genbank | 4314418 |
| Sequence 7849 | AC006512 | Genbank | 4926863 |
| Sequence 7850 | AC006987 | Genbank | 5123989 |
| Sequence 7851 | AC007022 | Genbank | 4753292 |
| Sequence 7852 | AC007040 | Genbank | 4371305 |
| Sequence 7853 | AC007172 | Genbank | 4731066 |
| Sequence 7854 | AC007308 | Genbank | 5903110 |
| Sequence 7855 | AC007347 | Genbank | 6806840 |
| Sequence 7856 | AC007381 | Genbank | 6604532 |
| Sequence 7857 | AC007393 | Genbank | 5306300 |
| Sequence 7858 | AC007401 | Genbank | 6560920 |
| Sequence 7859 | AC007617 | Genbank | 5230397 |
| Sequence 7860 | AC007681 | Genbank | 5836156 |
| Sequence 7861 | AC007686 | Genbank | 6466519 |
| Sequence 7862 | AC007688 | Genbank | 5815499 |
| Sequence 7863 | AC007842 | Genbank | 5080755 |
| Sequence 7864 | AC007845 | Genbank | 7109495 |
| Sequence 7865 | AC008041 | Genbank | 5649181 |
| Sequence 7866 | AC008132 | Genbank | 7025879 |
| Sequence 7867 | AC008134 | Genbank | 5801655 |
| Sequence 7868 | AC008990 | Genbank | 7209394 |
| Sequence 7869 | AC009330 | Genbank | 6553960 |
| Sequence 7870 | AC009514 | Genbank | 6016667 |
| Sequence 7871 | AC010077 | Genbank | 5870275 |
| Sequence 7872 | AC011311 | Genbank | 6850247 |
| Sequence 7873 | AC012085 | Genbank | 6634755 |
| Sequence 7874 | AC016026 | Genbank | 7940357 |
| Sequence 7875 | AF000364 | Genbank | 2697102 |
| Sequence 7876 | AF001433 | Genbank | 2529704 |
| Sequence 7877 | AF001435 | Genbank | 2529708 |
| Sequence 7878 | AF001687 | Genbank | 2653735 |
| Sequence 7879 | AF001977 | Genbank | 2914738 |
| Sequence 7880 | AF002020 | Genbank | 2276462 |
| Sequence 7881 | AF002163 | Genbank | 2290769 |
| Sequence 7882 | AF002223 | Genbank | 4079670 |
| Sequence 7883 | AF002697 | Genbank | 2511528 |
| Sequence 7884 | AF002715 | Genbank | 2352276 |
| Sequence 7885 | AF002994 | Genbank | 2121297 |
| Sequence 7886 | AF002996 | Genbank | 2121299 |
| Sequence 7887 | AF004562 | Genbank | 3041872 |
| Sequence 7888 | AF005067 | Genbank | 6979018 |
| Sequence 7889 | AF005258 | Genbank | 2627428 |
| Sequence 7890 | AF005361 | Genbank | 2343115 |
| Sequence 7891 | AF006043 | Genbank | 2674061 |
| Sequence 7892 | AF006083 | Genbank | 2282031 |
| Sequence 7893 | AF006087 | Genbank | 2282039 |
| Sequence 7894 | AF005515 | Genbank | 2645432 |
| Sequence 7895 | AF007170 | Genbank | 2865251 |
| Sequence 7896 | AF007544 | Genbank | 2970122 |
| Sequence 7897 | AF007791 | Genbank | 3779196 |
| Sequence 7898 | AF007871 | Genbank | 2358278 |
| Sequence 7899 | AF008303 | Genbank | 2580619 |
| Sequence 7900 | AF010187 | Genbank | 7025910 |
| Sequence 7901 | AF012872 | Genbank | 2326226 |
| Sequence 7902 | AF013970 | Genbank | 2801421 |
| Sequence 7903 | AF014398 | Genbank | 8148061 |
| Sequence 7904 | AF015044 | Genbank | 4102712 |
| Sequence 7905 | AF015913 | Genbank | 2323409 |
| Sequence 7906 | AF016050 | Genbank | 2978559 |
| Sequence 7907 | AF016266 | Genbank | 2529562 |
| Sequence 7908 | AF017790 | Genbank | 2501872 |
| Sequence 7909 | AF020038 | Genbank | 3641397 |
| Sequence 7910 | AF020736 | Genbank | 3450954 |
| Sequence 7911 | AF020833 | Genbank | 2460199 |
| Sequence 7912 | AF021336 | Genbank | 2541972 |
| Sequence 7913 | AF021351 | Genbank | 2460207 |
| Sequence 7914 | AF022229 | Genbank | 2809382 |
| Sequence 7915 | AF023268 | Genbank | 2564910 |
| Sequence 7916 | AF023462 | Genbank | 2564670 |
| Sequence 7917 | AF023676 | Genbank | 3211721 |
| Sequence 7918 | AF025439 | Genbank | 2815605 |
| Sequence 7919 | AF025999 | Genbank | 2570853 |
| Sequence 7920 | AF026166 | Genbank | 4090928 |
| Sequence 7921 | AF026292 | Genbank | 2559009 |
| Sequence 7922 | AF026548 | Genbank | 2583172 |
| Sequence 7923 | AF027299 | Genbank | 2739095 |
| Sequence 7924 | AF027302 | Genbank | 2522533 |
| Sequence 7925 | AF029308 | Genbank | 2564750 |
| Sequence 7926 | AF031416 | Genbank | 3213216 |
| Sequence 7927 | AF032456 | Genbank | 3004908 |
| Sequence 7928 | AF034373 | Genbank | 3820483 |
| Sequence 7929 | AF034756 | Genbank | 2654138 |
| Sequence 7930 | AF034759 | Genbank | 2653648 |
| Sequence 7931 | AF035191 | Genbank | 4104352 |
| Sequence 7932 | AF035286 | Genbank | 2661038 |
| Sequence 7933 | AF035295 | Genbank | 2661050 |
| Sequence 7934 | AF035296 | Genbank | 2661052 |
| Sequence 7935 | AF036536 | Genbank | 4104518 |
| Sequence 7936 | AF036682 | Genbank | 2707821 |
| Sequence 7937 | AF037448 | Genbank | 3037012 |
| Sequence 7938 | AF037643 | Genbank | 2746542 |
| Sequence 7939 | AF038196 | Genbank | 2795917 |
| Sequence 7940 | AF038318 | Genbank | 2708698 |
| Sequence 7941 | AF038391 | Genbank | 3123905 |
| Sequence 7942 | AF038392 | Genbank | 3123907 |
| Sequence 7943 | AF038554 | Genbank | 2895558 |
| Sequence 7944 | AF039703 | Genbank | 2789655 |
| Sequence 7945 | AF040707 | Genbank | 3688794 |
| Sequence 7946 | AF040966 | Genbank | 2792367 |
| Sequence 7947 | AF041004 | Genbank | 4335800 |
| Sequence 7948 | AF041248 | Genbank | 3243117 |
| Sequence 7949 | AF041259 | Genbank | 3335396 |
| Sequence 7950 | AF041381 | Genbank | 3080766 |
| Sequence 7951 | AF041483 | Genbank | 3493528 |
| Sequence 7952 | AF042084 | Genbank | 2792517 |
| Sequence 7953 | AF042166 | Genbank | 3298596 |
| Sequence 7954 | AF042379 | Genbank | 2801700 |
| Sequence 7955 | AF043045 | Genbank | 3282770 |
| Sequence 7956 | AF043324 | Genbank | 3005062 |
| Sequence 7957 | AF044127 | Genbank | 4105189 |
| Sequence 7958 | AF044321 | Genbank | 3170263 |
| Sequence 7959 | AF044588 | Genbank | 2865520 |
| Sequence 7960 | AF044773 | Genbank | 3002950 |
| Sequence 7961 | AF044954 | Genbank | 4164441 |
| Sequence 7962 | AF044956 | Genbank | 5326827 |
| Sequence 7963 | AF045606 | Genbank | 8277249 |
| Sequence 7964 | AF046025 | Genbank | 4581063 |
| Sequence 7965 | AF047181 | Genbank | 2909853 |
| Sequence 7966 | AF047184 | Genbank | 2909859 |
| Sequence 7967 | AF047431 | Genbank | 5514630 |
| Sequence 7968 | AF047432 | Genbank | 2911479 |
| Sequence 7969 | AF047437 | Genbank | 3335129 |
| Sequence 7970 | AF047442 | Genbank | 3335139 |
| Sequence 7971 | AF047472 | Genbank | 2921872 |
| Sequence 7972 | AF047599 | Genbank | 2906227 |
| Sequence 7973 | AF049910 | Genbank | 3435156 |
| Sequence 7974 | AF050078 | Genbank | 3818465 |
| Sequence 7975 | AF050641 | Genbank | 5326822 |
| Sequence 7976 | AF051782 | Genbank | 2947237 |
| Sequence 7977 | AF052059 | Genbank | 6851088 |
| Sequence 7978 | AF052106 | Genbank | 3360413 |
| Sequence 7979 | AF052130 | Genbank | 3360439 |
| Sequence 7980 | AF052155 | Genbank | 3360466 |
| Sequence 7981 | AF052178 | Genbank | 3360489 |
| Sequence 7982 | AF052179 | Genbank | 3360490 |
| Sequence 7983 | AF052182 | Genbank | 3360494 |
| Sequence 7984 | AF052186 | Genbank | 3360498 |
| Sequence 7985 | AF052504 | Genbank | 6650080 |
| Sequence 7986 | AF053069 | Genbank | 3136151 |
| Sequence 7987 | AF053074 | Genbank | 3283044 |
| Sequence 7988 | AF054187 | Genbank | 4092059 |
| Sequence 7989 | AF054284 | Genbank | 4033734 |
| Sequence 7990 | AF054990 | Genbank | 3005703 |
| Sequence 7991 | AF055014 | Genbank | 3005738 |
| Sequence 7992 | AF055017 | Genbank | 3005743 |
| Sequence 7993 | AF055022 | Genbank | 3005750 |
| Sequence 7994 | AF055029 | Genbank | 3005759 |
| Sequence 7995 | AF057160 | Genbank | 3694919 |
| Sequence 7996 | AF058392 | Genbank | 3063679 |
| Sequence 7997 | AF058988 | Genbank | 3300089 |
| Sequence 7998 | AF059321 | Genbank | 3169563 |
| Sequence 7999 | AF061016 | Genbank | 3127126 |
| Sequence 8000 | AF061243 | Genbank | 3779243 |
| Sequence 8001 | AF061738 | Genbank | 4335940 |
| Sequence 8002 | AF061749 | Genbank | 3372676 |
| Sequence 8003 | AF062536 | Genbank | 3139076 |
| Sequence 8004 | AF063308 | Genbank | 4106355 |
| Sequence 8005 | AF064084 | Genbank | 3135668 |
| Sequence 8006 | AF064603 | Genbank | 3152659 |
| Sequence 8007 | AF065389 | Genbank | 3152702 |
| Sequence 8008 | AF067170 | Genbank | 4894373 |
| Sequence 8009 | AF067513 | Genbank | 3850305 |
| Sequence 8010 | AF067656 | Genbank | 3901271 |
| Sequence 8011 | AF067972 | Genbank | 4927369 |
| Sequence 8012 | AF068235 | Genbank | 4321975 |
| Sequence 8013 | AF068754 | Genbank | 3283408 |
| Sequence 8014 | AF069301 | Genbank | 3193335 |
| Sequence 8015 | AF069984 | Genbank | 3242977 |
| Sequence 8016 | AF070550 | Genbank | 3387913 |
| Sequence 8017 | AF070561 | Genbank | 3387928 |
| Sequence 8018 | AF070563 | Genbank | 3387931 |
| Sequence 8019 | AF070578 | Genbank | 3387950 |
| Sequence 8020 | AF070598 | Genbank | 3387976 |
| Sequence 8021 | AF070603 | Genbank | 3387983 |
| Sequence 8022 | AF070663 | Genbank | 4454701 |
| Sequence 8023 | AF071172 | Genbank | 5107833 |
| Sequence 8024 | AF071202 | Genbank | 3335172 |
| Sequence 8025 | AF071309 | Genbank | 3426319 |
| Sequence 8026 | AF071593 | Genbank | 3249712 |
| Sequence 8027 | AF071738 | Genbank | 4263875 |
| Sequence 8028 | AF071748 | Genbank | 3916211 |
| Sequence 8029 | AF072933 | Genbank | 5305428 |
| Sequence 8030 | AF073344 | Genbank | 5410229 |
| Sequence 8031 | AF074331 | Genbank | 5052074 |
| Sequence 8032 | AF075575 | Genbank | 3600027 |
| Sequence 8033 | AF076273 | Genbank | 4185718 |
| Sequence 8034 | AF076678 | Genbank | 4884560 |
| Sequence 8035 | AF076844 | Genbank | 4019216 |
| Sequence 8036 | AF077036 | Genbank | 4689119 |
| Sequence 8037 | AF077042 | Genbank | 4689131 |
| Sequence 8038 | AF077188 | Genbank | 5565654 |
| Sequence 8039 | AF077203 | Genbank | 4679019 |
| Sequence 8040 | AF077367 | Genbank | 3820534 |
| Sequence 8041 | AF078103 | Genbank | 4218944 |
| Sequence 8042 | AF078107 | Genbank | 3342907 |
| Sequence 8043 | AF078671 | Genbank | 4877563 |
| Sequence 8044 | AF078776 | Genbank | 3511274 |
| Sequence 8045 | AF078845 | Genbank | 5531804 |
| Sequence 8046 | AF078847 | Genbank | 5531808 |
| Sequence 8047 | AF078854 | Genbank | 5531822 |
| Sequence 8048 | AF078863 | Genbank | 5531840 |
| Sequence 8049 | AF078866 | Genbank | 5531846 |
| Sequence 8050 | AF080561 | Genbank | 3746786 |
| Sequence 8051 | AF081484 | Genbank | 3420928 |
| Sequence 8052 | AF083068 | Genbank | 4808551 |
| Sequence 8053 | AF083106 | Genbank | 7555470 |
| Sequence 8054 | AF083190 | Genbank | 3599414 |
| Sequence 8055 | AF083246 | Genbank | 5106786 |
| Sequence 8056 | AF083255 | Genbank | 3435311 |
| Sequence 8057 | AF085355 | Genbank | 5114044 |
| Sequence 8058 | AF085357 | Genbank | 5114048 |
| Sequence 8059 | AF085359 | Genbank | 5114052 |
| Sequence 8060 | AF086234 | Genbank | 3483579 |
| Sequence 8061 | AF086280 | Genbank | 3483625 |
| Sequence 8062 | AF086406 | Genbank | 3483751 |
| Sequence 8063 | AF087438 | Genbank | 3820591 |
| Sequence 8064 | AF087986 | Genbank | 3523192 |
| Sequence 8065 | AF088060 | Genbank | 3523266 |
| Sequence 8066 | AF089841 | Genbank | 4218954 |
| Sequence 8067 | AF090426 | Genbank | 3982903 |
| Sequence 8068 | AF090927 | Genbank | 6690220 |
| Sequence 8069 | AF091035 | Genbank | 6002584 |
| Sequence 8070 | AF091073 | Genbank | 3859983 |
| Sequence 8071 | AF092136 | Genbank | 5138921 |
| Sequence 8072 | AF092565 | Genbank | 3661609 |
| Sequence 8073 | AF093239 | Genbank | 3661615 |
| Sequence 8074 | AF094516 | Genbank | 3820613 |
| Sequence 8075 | AF095192 | Genbank | 4322819 |
| Sequence 8076 | AF095448 | Genbank | 4063889 |
| Sequence 8077 | AF095890 | Genbank | 4185790 |
| Sequence 8078 | AF096160 | Genbank | 4454849 |
| Sequence 8079 | AF097514 | Genbank | 4808600 |
| Sequence 8080 | AF098865 | Genbank | 4204674 |
| Sequence 8081 | AF098948 | Genbank | 5456746 |
| Sequence 8082 | AF100615 | Genbank | 4808624 |
| Sequence 8083 | AF100755 | Genbank | 5410295 |
| Sequence 8084 | AF100756 | Genbank | 5410297 |
| Sequence 8085 | AF103800 | Genbank | 6048967 |
| Sequence 8086 | AF104913 | Genbank | 3941723 |
| Sequence 8087 | AF105366 | Genbank | 5106522 |
| Sequence 8088 | AF106798 | Genbank | 5805207 |
| Sequence 8089 | AF107405 | Genbank | 5531903 |
| Sequence 8090 | AF107406 | Genbank | 5531905 |
| Sequence 8091 | AF110184 | Genbank | 5565858 |
| Sequence 8092 | AF111168 | Genbank | 4186181 |
| Sequence 8093 | AF111853 | Genbank | 6560625 |
| Sequence 8094 | AF112227 | Genbank | 4545218 |
| Sequence 8095 | AF113019 | Genbank | 6642761 |
| Sequence 8096 | AF113129 | Genbank | 6523820 |
| Sequence 8097 | AF113132 | Genbank | 5326801 |
| Sequence 8098 | AF113249 | Genbank | 6650673 |
| Sequence 8099 | AF113251 | Genbank | 5852417 |
| Sequence 8100 | AF113702 | Genbank | 6855636 |
| Sequence 8101 | AF116241 | Genbank | 5006438 |
| Sequence 8102 | AF117888 | Genbank | 5732617 |
| Sequence 8103 | AF118124 | Genbank | 4235636 |
| Sequence 8104 | AF124145 | Genbank | 5931954 |
| Sequence 8105 | AF125102 | Genbank | 5106999 |
| Sequence 8106 | AF125525 | Genbank | 4689281 |
| Sequence 8107 | AF126021 | Genbank | 6563273 |
| Sequence 8108 | AF126246 | Genbank | 4406523 |
| Sequence 8109 | AF126782 | Genbank | 6318547 |
| Sequence 8110 | AF126799 | Genbank | 4406527 |
| Sequence 8111 | AF127138 | Genbank | 5922724 |
| Sequence 8112 | AF127563 | Genbank | 4455124 |
| Sequence 8113 | AF127761 | Genbank | 4455128 |
| Sequence 8114 | AF127966 | Genbank | 5532271 |
| Sequence 8115 | AF128458 | Genbank | 7140573 |
| Sequence 8116 | AF129930 | Genbank | 4558509 |
| Sequence 8117 | AF131742 | Genbank | 4406554 |
| Sequence 8118 | AF131797 | Genbank | 4406625 |
| Sequence 8119 | AF131802 | Genbank | 4406633 |
| Sequence 8120 | AF131820 | Genbank | 4406655 |
| Sequence 8121 | AF131826 | Genbank | 4406662 |
| Sequence 8122 | AF132033 | Genbank | 5524202 |
| Sequence 8123 | AF132856 | Genbank | 4809025 |
| Sequence 8124 | AF132940 | Genbank | 4680650 |
| Sequence 8125 | AF132946 | Genbank | 4680662 |
| Sequence 8126 | AF132965 | Genbank | 4680700 |
| Sequence 8127 | AF134726 | Genbank | 4529886 |
| Sequence 8128 | AF134803 | Genbank | 4868362 |
| Sequence 8129 | AF136271 | Genbank | 4732145 |
| Sequence 8130 | AF136715 | Genbank | 5019773 |
| Sequence 8131 | AF140710 | Genbank | 6073848 |
| Sequence 8132 | AF141347 | Genbank | 4929133 |
| Sequence 8133 | AF141870 | Genbank | 5006601 |
| Sequence 8134 | AF143536 | Genbank | 4929219 |
| Sequence 8135 | AF145732 | Genbank | 5579330 |
| Sequence 8136 | AF147271 | Genbank | 5823152 |
| Sequence 8137 | AF147331 | Genbank | 4761682 |
| Sequence 8138 | AF147410 | Genbank | 4761761 |
| Sequence 8139 | AF147787 | Genbank | 5231122 |
| Sequence 8140 | AF148513 | Genbank | 4809268 |
| Sequence 8141 | AF149045 | Genbank | 6010479 |
| Sequence 8142 | AF150087 | Genbank | 5107162 |
| Sequence 8143 | AF151018 | Genbank | 7106757 |
| Sequence 8144 | AF151020 | Genbank | 7106761 |
| Sequence 8145 | AF151042 | Genbank | 7106805 |
| Sequence 8146 | AF151051 | Genbank | 7106823 |
| Sequence 8147 | AF151109 | Genbank | 6166337 |
| Sequence 8148 | AF151799 | Genbank | 4929550 |
| Sequence 8149 | AF151805 | Genbank | 4929562 |
| Sequence 8150 | AF151808 | Genbank | 4929568 |
| Sequence 8151 | AF151811 | Genbank | 4929574 |
| Sequence 8152 | AF151826 | Genbank | 4929604 |
| Sequence 8153 | AF151841 | Genbank | 4929634 |
| Sequence 8154 | AF151846 | Genbank | 4929644 |
| Sequence 8155 | AF151861 | Genbank | 4929674 |
| Sequence 8156 | AF151869 | Genbank | 4929690 |
| Sequence 8157 | AF151886 | Genbank | 4929724 |
| Sequence 8158 | AF151902 | Genbank | 4929756 |
| Sequence 8159 | AF151904 | Genbank | 4929760 |
| Sequence 8160 | AF152961 | Genbank | 5499740 |
| Sequence 8161 | AF155095 | Genbank | 5360084 |
| Sequence 8162 | AF155110 | Genbank | 5360114 |
| Sequence 8163 | AF155238 | Genbank | 9186822 |
| Sequence 8164 | AF156165 | Genbank | 7145093 |
| Sequence 8165 | AF157378 | Genbank | 5714631 |
| Sequence 8166 | AF159295 | Genbank | 5714635 |
| Sequence 8167 | AF161371 | Genbank | 6841155 |
| Sequence 8168 | AF161417 | Genbank | 6841247 |
| Sequence 8169 | AF161448 | Genbank | 6841309 |
| Sequence 8170 | AF161455 | Genbank | 6841323 |
| Sequence 8171 | AF161458 | Genbank | 6841439 |
| Sequence 8172 | AF161485 | Genbank | 6841493 |
| Sequence 8173 | AF161491 | Genbank | 6841505 |
| Sequence 8174 | AF161493 | Genbank | 6841509 |
| Sequence 8175 | AF161511 | Genbank | 6841545 |
| Sequence 8176 | AF161530 | Genbank | 6841583 |
| Sequence 8177 | AF163441 | Genbank | 6969979 |
| Sequence 8178 | AF165967 | Genbank | 5802826 |
| Sequence 8179 | AF167572 | Genbank | 6164703 |
| Sequence 8180 | AF167706 | Genbank | 6979310 |
| Sequence 8181 | AF168787 | Genbank | 7239175 |
| Sequence 8182 | AF168956 | Genbank | 5702387 |
| Sequence 8183 | AF180682 | Genbank | 6684524 |
| Sequence 8184 | AF181265 | Genbank | 7212810 |
| Sequence 8185 | AF182316 | Genbank | 6731234 |
| Sequence 8186 | AF187320 | Genbank | 6164847 |
| Sequence 8187 | AF187554 | Genbank | 6663225 |
| Sequence 8188 | AF190167 | Genbank | 6456117 |
| Sequence 8189 | AF190725 | Genbank | 6118538 |
| Sequence 8190 | AF191339 | Genbank | 6180012 |
| Sequence 8191 | AF191340 | Genbank | 6180014 |
| Sequence 8192 | AF192968 | Genbank | 6449082 |
| Sequence 8193 | AF196779 | Genbank | 6180170 |
| Sequence 8194 | AF196969 | Genbank | 6289073 |
| Sequence 8195 | AF198444 | Genbank | 7025338 |
| Sequence 8196 | AF198487 | Genbank | 6941955 |
| Sequence 8197 | AF199596 | Genbank | 6842049 |
| Sequence 8198 | AF203815 | Genbank | 6979641 |
| Sequence 8199 | AF203978 | Genbank | 6503203 |
| Sequence 8200 | AF207907 | Genbank | 6653648 |
| Sequence 8201 | AF212162 | Genbank | 6665785 |
| Sequence 8202 | AF220417 | Genbank | 7678700 |
| Sequence 8203 | AF223408 | Genbank | 6911266 |
| Sequence 8204 | AF224669 | Genbank | 7012905 |
| Sequence 8205 | AF227218 | Genbank | 7159279 |
| Sequence 8206 | AJ000186 | Genbank | 2463195 |
| Sequence 8207 | AJ000332 | Genbank | 2274967 |
| Sequence 8208 | AJ000644 | Genbank | 2695707 |
| Sequence 8209 | AJ001050 | Genbank | 2832345 |
| Sequence 8210 | AJ001443 | Genbank | 6006514 |
| Sequence 8211 | AJ002955 | Genbank | 2632122 |
| Sequence 8212 | AJ004832 | Genbank | 2982500 |
| Sequence 8213 | AJ006291 | Genbank | 3805937 |
| Sequence 8214 | AJ007398 | Genbank | 3668140 |
| Sequence 8215 | AJ007509 | Genbank | 3319955 |
| Sequence 8216 | AJ007670 | Genbank | 3560123 |
| Sequence 8217 | AJ010597 | Genbank | 3559873 |
| Sequence 8218 | AJ011930 | Genbank | 3859769 |
| Sequence 8219 | AJ012463 | Genbank | 3850049 |
| Sequence 8220 | AJ012499 | Genbank | 5441359 |
| Sequence 8221 | AJ131186 | Genbank | 5689737 |
| Sequence 8222 | AJ223366 | Genbank | 6691913 |
| Sequence 8223 | AJ224442 | Genbank | 2911586 |
| Sequence 8224 | AJ245416 | Genbank | 5701851 |
| Sequence 8225 | AJ250475 | Genbank | 8919097 |
| Sequence 8226 | AJ275986 | Genbank | 7228110 |
| Sequence 8227 | AK000056 | Genbank | 7019892 |
| Sequence 8228 | AK000161 | Genbank | 7020067 |
| Sequence 8229 | AK000181 | Genbank | 7020098 |
| Sequence 8230 | AK000221 | Genbank | 7020163 |
| Sequence 8231 | AK000228 | Genbank | 7020174 |
| Sequence 8232 | AK000260 | Genbank | 7020221 |
| Sequence 8233 | AK000284 | Genbank | 7020260 |
| Sequence 8234 | AK000286 | Genbank | 7020264 |
| Sequence 8235 | AK000298 | Genbank | 7020286 |
| Sequence 8236 | AK000301 | Genbank | 7020291 |
| Sequence 8237 | AK000357 | Genbank | 7020388 |
| Sequence 8238 | AK000361 | Genbank | 7020395 |
| Sequence 8239 | AK000371 | Genbank | 7020413 |
| Sequence 8240 | AK000373 | Genbank | 7020417 |
| Sequence 8241 | AK000398 | Genbank | 7020459 |
| Sequence 8242 | AK000407 | Genbank | 7020474 |
| Sequence 8243 | AK000436 | Genbank | 7020522 |
| Sequence 8244 | AK000474 | Genbank | 7020586 |
| Sequence 8245 | AK000498 | Genbank | 7020628 |
| Sequence 8246 | AK000505 | Genbank | 7020640 |
| Sequence 8247 | AK000543 | Genbank | 7020709 |
| Sequence 8248 | AK000544 | Genbank | 7020710 |
| Sequence 8249 | AK000558 | Genbank | 7020734 |
| Sequence 8250 | AK000560 | Genbank | 7020738 |
| Sequence 8251 | AK000595 | Genbank | 7020796 |
| Sequence 8252 | AK000630 | Genbank | 7020848 |
| Sequence 8253 | AK000651 | Genbank | 7020882 |
| Sequence 8254 | AK000664 | Genbank | 7020901 |
| Sequence 8255 | AK000669 | Genbank | 7020909 |
| Sequence 8256 | AK000714 | Genbank | 7020975 |
| Sequence 8257 | AK000739 | Genbank | 7021014 |
| Sequence 8258 | AK000740 | Genbank | 7021016 |
| Sequence 8259 | AK000745 | Genbank | 7021025 |
| Sequence 8260 | AK000757 | Genbank | 7021043 |
| Sequence 8261 | AK000759 | Genbank | 7021046 |
| Sequence 8262 | AK000799 | Genbank | 7021103 |
| Sequence 8263 | AK000859 | Genbank | 7021183 |
| Sequence 8264 | AK000946 | Genbank | 7021928 |
| Sequence 8265 | AK000982 | Genbank | 7021980 |
| Sequence 8266 | AK000990 | Genbank | 7021992 |
| Sequence 8267 | AK001090 | Genbank | 7022139 |
| Sequence 8268 | AK001112 | Genbank | 7022170 |
| Sequence 8269 | AK001123 | Genbank | 7022186 |
| Sequence 8270 | AK001184 | Genbank | 7022279 |
| Sequence 8271 | AK001188 | Genbank | 7022286 |
| Sequence 8272 | AK001262 | Genbank | 7022406 |
| Sequence 8273 | AK001264 | Genbank | 7022409 |
| Sequence 8274 | AK001269 | Genbank | 7022419 |
| Sequence 8275 | AK001277 | Genbank | 7022431 |
| Sequence 8276 | AK001281 | Genbank | 7022437 |
| Sequence 8277 | AK001288 | Genbank | 7022448 |
| Sequence 8278 | AK001292 | Genbank | 7022455 |
| Sequence 8279 | AK001313 | Genbank | 7022490 |
| Sequence 8280 | AK001330 | Genbank | 7022520 |
| Sequence 8281 | AK001344 | Genbank | 7022543 |
| Sequence 8282 | AK001363 | Genbank | 7022575 |
| Sequence 8283 | AK001364 | Genbank | 7022577 |
| Sequence 8284 | AK001366 | Genbank | 7022579 |
| Sequence 8285 | AK001387 | Genbank | 7022615 |
| Sequence 8286 | AK001392 | Genbank | 7022621 |
| Sequence 8287 | AK001395 | Genbank | 7022625 |
| Sequence 8288 | AK001410 | Genbank | 7022649 |
| Sequence 8289 | AK001436 | Genbank | 7022692 |
| Sequence 8290 | AK001478 | Genbank | 7022760 |
| Sequence 8291 | AK001492 | Genbank | 7022781 |
| Sequence 8292 | AK001508 | Genbank | 7022805 |
| Sequence 8293 | AK001521 | Genbank | 7022828 |
| Sequence 8294 | AK001539 | Genbank | 7022856 |
| Sequence 8295 | AK001565 | Genbank | 7022895 |
| Sequence 8296 | AK001601 | Genbank | 7022955 |
| Sequence 8297 | AK001613 | Genbank | 7022975 |
| Sequence 8298 | AK001627 | Genbank | 7022996 |
| Sequence 8299 | AK001636 | Genbank | 7023010 |
| Sequence 8300 | AK001675 | Genbank | 7023079 |
| Sequence 8301 | AK001690 | Genbank | 7023105 |
| Sequence 8302 | AK001691 | Genbank | 7023106 |
| Sequence 8303 | AK001700 | Genbank | 7023122 |
| Sequence 8304 | Ak001714 | Genbank | 7023146 |
| Sequence 8305 | AK001716 | Genbank | 7023150 |
| Sequence 8306 | AK001725 | Genbank | 7023165 |
| Sequence 8307 | AK001741 | Genbank | 7023192 |
| Sequence 8308 | AK001775 | Genbank | 7023255 |
| Sequence 8309 | AK001829 | Genbank | 7023339 |
| Sequence 8310 | AK001852 | Genbank | 7023376 |
| Sequence 8311 | AK001939 | Genbank | 7023513 |
| Sequence 8312 | AK001962 | Genbank | 7023551 |
| Sequence 8313 | AK002019 | Genbank | 7023647 |
| Sequence 8314 | AK002051 | Genbank | 7023698 |
| Sequence 8315 | AK002060 | Genbank | 7023713 |
| Sequence 8316 | AK002094 | Genbank | 7023767 |
| Sequence 8317 | AK002136 | Genbank | 7023830 |
| Sequence 8318 | AK002165 | Genbank | 7023876 |
| Sequence 8319 | AK002212 | Genbank | 7023953 |
| Sequence 8320 | AL008639 | Genbank | 6599068 |
| Sequence 8321 | AL008721 | Genbank | 3171883 |
| Sequence 8322 | AL020995 | Genbank | 5881341 |
| Sequence 8323 | AL021453 | Genbank | 3413288 |
| Sequence 8324 | AL021546 | Genbank | 2826890 |
| Sequence 8325 | AL021707 | Genbank | 4582132 |
| Sequence 8326 | AL021807 | Genbank | 9367202 |
| Sequence 8327 | AL021917 | Genbank | 9367203 |
| Sequence 8328 | AL021978 | Genbank | 3135963 |
| Sequence 8329 | AL022157 | Genbank | 3319676 |
| Sequence 8330 | AL022313 | Genbank | 4200326 |
| Sequence 8331 | AL022318 | Genbank | 4826439 |
| Sequence 8332 | AL022339 | Genbank | 3355600 |
| Sequence 8333 | AL022394 | Genbank | 9581758 |
| Sequence 8334 | AL023803 | Genbank | 9408726 |
| Sequence 8335 | AL024498 | Genbank | 5924006 |
| Sequence 8336 | AL031387 | Genbank | 4493578 |
| Sequence 8337 | AL031427 | Genbank | 4835258 |
| Sequence 8338 | AL031680 | Genbank | 9368417 |
| Sequence 8339 | AL031685 | Genbank | 9368423 |
| Sequence 8340 | AL031733 | Genbank | 4826495 |
| Sequence 8341 | AL031778 | Genbank | 4153958 |
| Sequence 8342 | AL034343 | Genbank | 5931807 |
| Sequence 8343 | AL034379 | Genbank | 5918013 |
| Sequence 8344 | AL034380 | Genbank | 7981257 |
| Sequence 8345 | AL035420 | Genbank | 5918161 |
| Sequence 8346 | AL035551 | Genbank | 4826429 |
| Sequence 8347 | AL035587 | Genbank | 6002306 |
| Sequence 8348 | AL035659 | Genbank | 6562059 |
| Sequence 8349 | AL035683 | Genbank | 7288039 |
| Sequence 8350 | AL049219 | Genbank | 4499949 |
| Sequence 8351 | AL049367 | Genbank | 4500158 |
| Sequence 8352 | AL049539 | Genbank | 6456855 |
| Sequence 8353 | AL049546 | Genbank | 4678775 |
| Sequence 8354 | AL049589 | Genbank | 5679567 |
| Sequence 8355 | AL049611 | Genbank | 5881343 |
| Sequence 8356 | AL049646 | Genbank | 9368462 |
| Sequence 8357 | AL049712 | Genbank | 5629919 |
| Sequence 8358 | AL049713 | Genbank | 6006528 |
| Sequence 8359 | AL049742 | Genbank | 5419783 |
| Sequence 8360 | AL049795 | Genbank | 6010175 |
| Sequence 8361 | AL049801 | Genbank | 4902677 |
| Sequence 8362 | AL049821 | Genbank | 5791513 |
| Sequence 8363 | AL049943 | Genbank | 4884187 |
| Sequence 8364 | AL050011 | Genbank | 4884080 |
| Sequence 8365 | AL050028 | Genbank | 4884267 |
| Sequence 8366 | AL050107 | Genbank | 4884135 |
| Sequence 8367 | AL050118 | Genbank | 4884143 |
| Sequence 8368 | AL050135 | Genbank | 4884344 |
| Sequence 8369 | AL050187 | Genbank | 4884402 |
| Sequence 8370 | AL050208 | Genbank | 4884447 |
| Sequence 8371 | AL050274 | Genbank | 4886452 |
| Sequence 8372 | AL050332 | Genbank | 6010176 |
| Sequence 8373 | AL050367 | Genbank | 4914600 |
| Sequence 8374 | AL050396 | Genbank | 4914618 |
| Sequence 8375 | AL078581 | Genbank | 6967288 |
| Sequence 8376 | AL078594 | Genbank | 7161750 |
| Sequence 8377 | AL078611 | Genbank | 5019312 |
| Sequence 8378 | AL078621 | Genbank | 6013067 |
| Sequence 8379 | AL079298 | Genbank | 5102736 |
| Sequence 8380 | AL079352 | Genbank | 7799779 |
| Sequence 8381 | AL080080 | Genbank | 5262491 |
| Sequence 8382 | AL080155 | Genbank | 5262612 |
| Sequence 8383 | AL080157 | Genbank | 5262616 |
| Sequence 8384 | AL080172 | Genbank | 5262642 |
| Sequence 8385 | AL096738 | Genbank | 5419870 |
| Sequence 8386 | AL096740 | Genbank | 5419896 |
| Sequence 8387 | AL096752 | Genbank | 5419888 |
| Sequence 8388 | AL096791 | Genbank | 6066332 |
| Sequence 8389 | AL109788 | Genbank | 5725476 |
| Sequence 8390 | AL109794 | Genbank | 5725481 |
| Sequence 8391 | AL109925 | Genbank | 7105935 |
| Sequence 8392 | AL110244 | Genbank | 5817191 |
| Sequence 8393 | AL110268 | Genbank | 5817041 |
| Sequence 8394 | AL110273 | Genbank | 5817091 |
| Sequence 8395 | AL117398 | Genbank | 5911918 |
| Sequence 8396 | AL117409 | Genbank | 5912046 |
| Sequence 8397 | AL117429 | Genbank | 5911863 |
| Sequence 8398 | AL117482 | Genbank | 5911960 |
| Sequence 8399 | AL117500 | Genbank | 5912005 |
| Sequence 8400 | AL117572 | Genbank | 5912124 |
| Sequence 8401 | AL117573 | Genbank | 5912125 |
| Sequence 8402 | AL117579 | Genbank | 5912135 |
| Sequence 8403 | AL117594 | Genbank | 5912158 |
| Sequence 8404 | AL117607 | Genbank | 5912177 |
| Sequence 8405 | AL117694 | Genbank | 8248721 |
| Sequence 8406 | AL121575 | Genbank | 7264053 |
| Sequence 8407 | AL121790 | Genbank | 8919824 |
| Sequence 8408 | AL122020 | Genbank | 8176920 |
| Sequence 8409 | AL133036 | Genbank | 6453441 |
| Sequence 8410 | AL133064 | Genbank | 6453490 |
| Sequence 8411 | AL133078 | Genbank | 6453522 |
| Sequence 8412 | AL133215 | Genbank | 7228177 |
| Sequence 8413 | AL133246 | Genbank | 7594587 |
| Sequence 8414 | AL133353 | Genbank | 6706037 |
| Sequence 8415 | AL133455 | Genbank | 7019734 |
| Sequence 8416 | AL133555 | Genbank | 6599122 |
| Sequence 8417 | AL133583 | Genbank | 6599167 |
| Sequence 8418 | AL133589 | Genbank | 6599176 |
| Sequence 8419 | AL133609 | Genbank | 6599219 |
| Sequence 8420 | AL135744 | Genbank | 6682291 |
| Sequence 8421 | AL135858 | Genbank | 7159622 |
| Sequence 8422 | AL136295 | Genbank | 6850939 |
| Sequence 8423 | AL136543 | Genbank | 6807646 |
| Sequence 8424 | AL137263 | Genbank | 6807692 |
| Sequence 8425 | AL137304 | Genbank | 6807768 |
| Sequence 8426 | AL137327 | Genbank | 6807818 |
| Sequence 8427 | AL137406 | Genbank | 6807955 |
| Sequence 8428 | AL137471 | Genbank | 6808079 |
| Sequence 8429 | AL137540 | Genbank | 6808218 |
| Sequence 8430 | AL137548 | Genbank | 6808231 |
| Sequence 8431 | AL137585 | Genbank | 6808304 |
| Sequence 8432 | AL137641 | Genbank | 6808443 |
| Sequence 8433 | AL137681 | Genbank | 6807931 |
| Sequence 8434 | AL137683 | Genbank | 6807935 |
| Sequence 8435 | AL137727 | Genbank | 6808256 |
| Sequence 8436 | AL137743 | Genbank | 6808323 |
| Sequence 8437 | AL139165 | Genbank | 6969146 |
| Sequence 8438 | AP000010 | Genbank | 4666256 |
| Sequence 8439 | AP000046 | Genbank | 3132356 |
| Sequence 8440 | AP000066 | Genbank | 4579987 |
| Sequence 8441 | AP000493 | Genbank | 5926660 |
| Sequence 8442 | AP000501 | Genbank | 5926688 |
| Sequence 8443 | AP000529 | Genbank | 5931507 |
| Sequence 8444 | AP000694 | Genbank | 6693626 |
| Sequence 8445 | AP001115 | Genbank | 6899854 |
| Sequence 8446 | AP001137 | Genbank | 6970361 |
| Sequence 8447 | D00591 | Genbank | 220051 |
| Sequence 8448 | D01038 | Genbank | 220140 |
| Sequence 8449 | D10040 | Genbank | 219899 |
| Sequence 8450 | D10495 | Genbank | 520586 |
| Sequence 8451 | D12981 | Genbank | 219979 |
| Sequence 8452 | D13119 | Genbank | 285909 |
| Sequence 8453 | D13286 | Genbank | 496368 |
| Sequence 8454 | D13540 | Genbank | 220071 |
| Sequence 8455 | D13627 | Genbank | 286010 |
| Sequence 8456 | D13635 | Genbank | 285982 |
| Sequence 8457 | D13748 | Genbank | 219402 |
| Sequence 8458 | D14662 | Genbank | 285948 |
| Sequence 8459 | D14689 | Genbank | 285956 |
| Sequence 8460 | D14694 | Genbank | 603801 |
| Sequence 8461 | D14811 | Genbank | 285966 |
| Sequence 8462 | D16307 | Genbank | 303594 |
| Sequence 8463 | D16562 | Genbank | 506336 |
| Sequence 8464 | D21063 | Genbank | 434752 |
| Sequence 8465 | D21163 | Genbank | 434758 |
| Sequence 8466 | D21235 | Genbank | 498145 |
| Sequence 8467 | D21254 | Genbank | 575577 |
| Sequence 8468 | D21261 | Genbank | 434762 |
| Sequence 8469 | D21262 | Genbank | 434764 |
| Sequence 8470 | D21852 | Genbank | 434768 |
| Sequence 8471 | D25215 | Genbank | 517114 |
| Sequence 8472 | D26156 | Genbank | 505087 |
| Sequence 8473 | D26181 | Genbank | 473712 |
| Sequence 8474 | D26351 | Genbank | 450470 |
| Sequence 8475 | D26485 | Genbank | 468934 |
| Sequence 8476 | D28473 | Genbank | 551621 |
| Sequence 8477 | D28476 | Genbank | 460710 |
| Sequence 8478 | D28589 | Genbank | 460714 |
| Sequence 8479 | D29954 | Genbank | 473940 |
| Sequence 8480 | D29958 | Genbank | 473948 |
| Sequence 8481 | D30756 | Genbank | 488500 |
| Sequence 8482 | D31762 | Genbank | 498149 |
| Sequence 8483 | D31764 | Genbank | 498153 |
| Sequence 8484 | D31767 | Genbank | 505091 |
| Sequence 8485 | D31885 | Genbank | 505097 |
| Sequence 8486 | D31888 | Genbank | 506340 |
| Sequence 8487 | D37766 | Genbank | 2429078 |
| Sequence 8488 | D38047 | Genbank | 1037163 |
| Sequence 8489 | D38048 | Genbank | 1531532 |
| Sequence 8490 | D38073 | Genbank | 862331 |
| Sequence 8491 | D38522 | Genbank | 559331 |
| Sequence 8492 | D38524 | Genbank | 633070 |
| Sequence 8493 | D38549 | Genbank | 559702 |
| Sequence 8494 | D38553 | Genbank | 559714 |
| Sequence 8495 | D38555 | Genbank | 559716 |
| Sequence 8496 | D38616 | Genbank | 1304117 |
| Sequence 8497 | D42047 | Genbank | 577306 |
| Sequence 8498 | D42055 | Genbank | 577312 |
| Sequence 8499 | D42085 | Genbank | 577316 |
| Sequence 8500 | D43682 | Genbank | 1060913 |
| Sequence 8501 | D43949 | Genbank | 603952 |
| Sequence 8502 | D43950 | Genbank | 603954 |
| Sequence 8503 | D43951 | Genbank | 603956 |
| Sequence 8504 | D45021 | Genbank | 624872 |
| Sequence 8505 | D45131 | Genbank | 1304103 |
| Sequence 8506 | D49489 | Genbank | 1136742 |
| Sequence 8507 | D49490 | Genbank | 1072306 |
| Sequence 8508 | D50369 | Genbank | 2605589 |
| Sequence 8509 | D50372 | Genbank | 2605593 |
| Sequence 8510 | D50923 | Genbank | 1469188 |
| Sequence 8511 | D50929 | Genbank | 1469200 |
| Sequence 8512 | D55716 | Genbank | 1255616 |
| Sequence 8513 | D63475 | Genbank | 1665724 |
| Sequence 8514 | D63478 | Genbank | 1469869 |
| Sequence 8515 | 63479G | enbank | 6633998 |
| Sequence 8516 | D63486 | Genbank | 1469885 |
| Sequence 8517 | D63780 | Genbank | 2196444 |
| Sequence 8518 | D63861 | Genbank | 1769811 |
| Sequence 8519 | D63875 | Genbank | 961441 |
| Sequence 8520 | D78014 | Genbank | 1330241 |
| Sequence 8521 | D79205 | Genbank | 1754620 |
| Sequence 8522 | D79984 | Genbank | 1136385 |
| Sequence 8523 | D79991 | Genbank | 1136397 |
| Sequence 8524 | D80000 | Genbank | 1136415 |
| Sequence 8525 | D80009 | Genbank | 1136433 |
| Sequence 8526 | D80010 | Genbank | 1136435 |
| Sequence 8527 | D82348 | Genbank | 1311461 |
| Sequence 8528 | D83077 | Genbank | 1304131 |
| Sequence 8529 | D83778 | Genbank | 1228038 |
| Sequence 8530 | D83781 | Genbank | 1228044 |
| Sequence 8531 | D83782 | Genbank | 1228046 |
| Sequence 8532 | D84064 | Genbank | 2618587 |
| Sequence 8533 | D86198 | Genbank | 3062805 |
| Sequence 8534 | D86962 | Genbank | 1503997 |
| Sequence 8535 | D86965 | Genbank | 1504003 |
| Sequence 8536 | D86977 | Genbank | 1504027 |
| Sequence 8537 | D86978 | Genbank | 1504029 |
| Sequence 8538 | D86985 | Genbank | 6634002 |
| Sequence 8539 | D86987 | Genbank | 3869186 |
| Sequence 8540 | D87127 | Genbank | 1817551 |
| Sequence 8541 | D87436 | Genbank | 1665766 |
| Sequence 8542 | D87447 | Genbank | 1665782 |
| Sequence 8543 | D87452 | Genbank | 1665792 |
| Sequence 8544 | D87464 | Genbank | 1665812 |
| Sequence 8545 | D87470 | Genbank | 1665822 |
| Sequence 8546 | D87717 | Genbank | 1663709 |
| Sequence 8547 | D87735 | Genbank | 1620021 |
| Sequence 8548 | D88674 | Genbank | 2641951 |
| Sequence 8549 | D88687 | Genbank | 1843433 |
| Sequence 8550 | D89980 | Genbank | 2804272 |
| Sequence 8551 | D90391 | Genbank | 219491 |
| Sequence 8552 | E01094 | Genbank | 2169353 |
| Sequence 8553 | E01787 | Genbank | 2170040 |
| Sequence 8554 | E01816 | Genbank | 2170068 |
| Sequence 8555 | E01956 | Genbank | 2170204 |
| Sequence 8556 | E02164 | Genbank | 2170402 |
| Sequence 8557 | E02628 | Genbank | 2170856 |
| Sequence 8558 | E06721 | Genbank | 2174903 |
| Sequence 8559 | E12458 | Genbank | 3251291 |
| Sequence 8560 | E13330 | Genbank | 3252135 |
| Sequence 8561 | E14580 | Genbank | 5709263 |
| Sequence 8562 | E14872 | Genbank | 5709555 |
| Sequence 8563 | E15239 | Genbank | 5709922 |
| Sequence 8564 | E15695 | Genbank | 5710378 |
| Sequence 8565 | E17361 | Genbank | 5712044 |
| Sequence 8566 | J02642 | Genbank | 182862 |
| Sequence 8567 | J02923 | Genbank | 189501 |
| Sequence 8568 | J03007 | Genbank | 179211 |
| Sequence 8569 | J03015 | Genbank | 337755 |
| Sequence 8570 | J03202 | Genbank | 186916 |
| Sequence 8571 | J03473 | Genbank | 337423 |
| Sequence 8572 | J03518 | Genbank | 340389 |
| Sequence 8573 | J03528 | Genbank | 188671 |
| Sequence 8574 | J03575 | Genbank | 189737 |
| Sequence 8575 | J03576 | Genbank | 189753 |
| Sequence 8576 | J03592 | Genbank | 339722 |
| Sequence 8577 | J03799 | Genbank | 186840 |
| Sequence 8578 | J03827 | Genbank | 340418 |
| Sequence 8579 | J04031 | Genbank | 187464 |
| Sequence 8580 | J04088 | Genbank | 292829 |
| Sequence 8581 | J04164 | Genbank | 177801 |
| Sequence 8582 | J04173 | Genbank | 551173 |
| Sequence 8583 | J04444 | Genbank | 181239 |
| Sequence 8584 | J04543 | Genbank | 338243 |
| Sequence 8585 | J04611 | Genbank | 178649 |
| Sequence 8586 | J04615 | Genbank | 338246 |
| Sequence 8587 | J04988 | Genbank | 184422 |
| Sequence 8588 | J05594 | Genbank | 1203981 |
| Sequence 8589 | J05633 | Genbank | 186504 |
| Sequence 8590 | K00558 | Genbank | 340020 |
| Sequence 8591 | L00160 | Genbank | 189904 |
| Sequence 8592 | L00635 | Genbank | 292032 |
| Sequence 8593 | L01457 | Genbank | 179282 |
| Sequence 8594 | L04733 | Genbank | 307084 |
| Sequence 8595 | L05093 | Genbank | 401844 |
| Sequence 8596 | L05425 | Genbank | 179284 |
| Sequence 8597 | L06237 | Genbank | 437000 |
| Sequence 8598 | L06328 | Genbank | 340200 |
| Sequence 8599 | L06419 | Genbank | 190073 |
| Sequence 8600 | L07044 | Genbank | 291894 |
| Sequence 8601 | L08238 | Genbank | 307198 |
| Sequence 8602 | L09159 | Genbank | 307374 |
| Sequence 8603 | L09561 | Genbank | 3192937 |
| Sequence 8604 | L11931 | Genbank | 307421 |
| Sequence 8605 | L11932 | Genbank | 307423 |
| Sequence 8606 | L13850 | Genbank | 292165 |
| Sequence 8607 | L14076 | Genbank | 307437 |
| Sequence 8608 | L14283 | Genbank | 307355 |
| Sequence 8609 | L14599 | Genbank | 348238 |
| Sequence 8610 | L14837 | Genbank | 292937 |
| Sequence 8611 | L16783 | Genbank | 452438 |
| Sequence 8612 | L19597 | Genbank | 306467 |
| Sequence 8613 | L19956 | Genbank | 306456 |
| Sequence 8614 | L20941 | Genbank | 507251 |
| Sequence 8615 | L22005 | Genbank | 388308 |
| Sequence 8616 | L22009 | Genbank | 347313 |
| Sequence 8617 | L22740 | Genbank | 409222 |
| Sequence 8618 | L23959 | Genbank | 414316 |
| Sequence 8619 | L25270 | Genbank | 457136 |
| Sequence 8620 | L25876 | Genbank | 414584 |
| Sequence 8621 | L25880 | Genbank | 450272 |
| Sequence 8622 | L26524 | Genbank | 457232 |
| Sequence 8623 | L27211 | Genbank | 558656 |
| Sequence 8624 | L27711 | Genbank | 808006 |
| Sequence 8625 | L29216 | Genbank | 632965 |
| Sequence 8626 | L29296 | Genbank | 559042 |
| Sequence 8627 | L29555 | Genbank | 522196 |
| Sequence 8628 | L34075 | Genbank | 508481 |
| Sequence 8629 | L36056 | Genbank | 561631 |
| Sequence 8630 | L36818 | Genbank | 556191 |
| Sequence 8631 | L38961 | Genbank | 624703 |
| Sequence 8632 | L39068 | Genbank | 994714 |
| Sequence 8633 | L39210 | Genbank | 1702963 |
| Sequence 8634 | L39211 | Genbank | 755645 |
| Sequence 8635 | L40326 | Genbank | 695361 |
| Sequence 8636 | L40586 | Genbank | 1222629 |
| Sequence 8637 | L40933 | Genbank | 1160964 |
| Sequence 8638 | L41607 | Genbank | 886293 |
| Sequence 8639 | L41816 | Genbank | 790789 |
| Sequence 8640 | L42379 | Genbank | 1203964 |
| Sequence 8641 | L42542 | Genbank | 974142 |
| Sequence 8642 | L42809 | Genbank | 1160966 |
| Sequence 8643 | L43509 | Genbank | 896282 |
| Sequence 8644 | L47162 | Genbank | 1082035 |
| Sequence 8645 | L47647 | Genbank | 1000861 |
| Sequence 8646 | L49345 | Genbank | 1405422 |
| Sequence 8647 | L54057 | Genbank | 1196416 |
| Sequence 8648 | L77701 | Genbank | 1280205 |
| Sequence 8649 | L78464 | Genbank | 1479980 |
| Sequence 8650 | L78669 | Genbank | 1374862 |
| Sequence 8651 | M11147 | Genbank | 182513 |
| Sequence 8652 | M11560 | Genbank | 178350 |
| Sequence 8653 | M12938 | Genbank | 182515 |
| Sequence 8654 | M13520 | Genbank | 179459 |
| Sequence 8655 | M13755 | Genbank | 184570 |
| Sequence 8656 | M14326 | Genbank | 340005 |
| Sequence 8657 | M14631 | Genbank | 183416 |
| Sequence 8658 | M15182 | Genbank | 183232 |
| Sequence 8659 | M16038 | Genbank | 187268 |
| Sequence 8660 | M16247 | Genbank | 178044 |
| Sequence 8661 | M16279 | Genbank | 188542 |
| Sequence 8662 | M17081 | Genbank | 178151 |
| Sequence 8663 | M17851 | Genbank | 182860 |
| Sequence 8664 | M17885 | Genbank | 190231 |
| Sequence 8665 | M17886 | Genbank | 190233 |
| Sequence 8666 | M18112 | Genbank | 190166 |
| Sequence 8667 | M18157 | Genbank | 186640 |
| Sequence 8668 | M18366 | Genbank | 179131 |
| Sequence 8669 | M19961 | Genbank | 180940 |
| Sequence 8670 | M20372 | Genbank | 189372 |
| Sequence 8671 | M20776 | Genbank | 180909 |
| Sequence 8672 | M21154 | Genbank | 178517 |
| Sequence 8673 | M21575 | Genbank | 1311702 |
| Sequence 8674 | M21895 | Genbank | 189523 |
| Sequence 8675 | M22382 | Genbank | 190126 |
| Sequence 8676 | M22538 | Genbank | 986883 |
| Sequence 8677 | M22803 | Genbank | 190379 |
| Sequence 8678 | M22919 | Genbank | 189016 |
| Sequence 8679 | M22920 | Genbank | 189021 |
| Sequence 8680 | M22960 | Genbank | 190282 |
| Sequence 8681 | M23254 | Genbank | 511636 |
| Sequence 8682 | M23419 | Genbank | 181996 |
| Sequence 8683 | M23725 | Genbank | 189997 |
| Sequence 8684 | M24194 | Genbank | 187701 |
| Sequence 8685 | M24355 | Genbank | 182604 |
| Sequence 8686 | M24486 | Genbank | 190785 |
| Sequence 8687 | M24543 | Genbank | 341200 |
| Sequence 8688 | M26066 | Genbank | 189854 |
| Sequence 8689 | M26325 | Genbank | 186688 |
| Sequence 8690 | M26663 | Genbank | 618463 |
| Sequence 8691 | M29366 | Genbank | 181979 |
| Sequence 8692 | M29551 | Genbank | 180708 |
| Sequence 8693 | M31159 | Genbank | 183115 |
| Sequence 8694 | M31511 | Genbank | 179907 |
| Sequence 8695 | M31606 | Genbank | 189940 |
| Sequence 8696 | M33308 | Genbank | 340236 |
| Sequence 8697 | M34055 | Genbank | 190791 |
| Sequence 8698 | M34064 | Genbank | 416292 |
| Sequence 8699 | M34081 | Genbank | 337458 |
| Sequence 8700 | M34088 | Genbank | 182122 |
| Sequence 8701 | M34668 | Genbank | 190738 |
| Sequence 8702 | M37499 | Genbank | 188886 |
| Sequence 8703 | M38188 | Genbank | 189378 |
| Sequence 8704 | M55153 | Genbank | 339520 |
| Sequence 8705 | M55618 | Genbank | 184483 |
| Sequence 8706 | M57567 | Genbank | 178986 |
| Sequence 8707 | M58028 | Genbank | 340071 |
| Sequence 8708 | M59371 | Genbank | 181943 |
| Sequence 8709 | M59849 | Genbank | 182591 |
| Sequence 8710 | M61715 | Genbank | 340367 |
| Sequence 8711 | M62810 | Genbank | 188563 |
| Sequence 8712 | M64098 | Genbank | 183891 |
| Sequence 8713 | M64571 | Genbank | 187382 |
| Sequence 8714 | M64788 | Genbank | 190855 |
| Sequence 8715 | M65217 | Genbank | 184404 |
| Sequence 8716 | M67480 | Genbank | 190364 |
| Sequence 8717 | M69066 | Genbank | 188625 |
| Sequence 8718 | M69181 | Genbank | 641957 |
| Sequence 8719 | M73816 | Genbank | 183609 |
| Sequence 8720 | M74491 | Genbank | 178161 |
| Sequence 8721 | M75126 | Genbank | 184020 |
| Sequence 8722 | M76729 | Genbank | 189519 |
| Sequence 8723 | M80254 | Genbank | 181273 |
| Sequence 8724 | M80335 | Genbank | 189966 |
| Sequence 8725 | M80646 | Genbank | 338701 |
| Sequence 8726 | M80776 | Genbank | 179334 |
| Sequence 8727 | M81934 | Genbank | 180172 |
| Sequence 8728 | M83651 | Genbank | 431032 |
| Sequence 8729 | M84326 | Genbank | 178163 |
| Sequence 8730 | M84739 | Genbank | 179881 |
| Sequence 8731 | M85038 | Genbank | 190189 |
| Sequence 8732 | M86492 | Genbank | 183369 |
| Sequence 8733 | M91670 | Genbank | 181915 |
| Sequence 8734 | M95627 | Genbank | 870802 |
| Sequence 8735 | M96684 | Genbank | 190749 |
| Sequence 8736 | M96803 | Genbank | 338442 |
| Sequence 8737 | M96995 | Genbank | 181975 |
| Sequence 8738 | M97501 | Genbank | 180621 |
| Sequence 8739 | M97934 | Genbank | 186566 |
| Sequence 8740 | M97935 | Genbank | 2281070 |
| Sequence 8741 | M98478 | Genbank | 339577 |
| Sequence 8742 | M99422 | Genbank | 183662 |
| Sequence 8743 | S60710 | Genbank | 300301 |
| Sequence 8744 | S62138 | Genbank | 386158 |
| Sequence 8745 | S67779 | Genbank | 459372 |
| Sequence 8746 | S69272 | Genbank | 546087 |
| Sequence 8747 | S70114 | Genbank | 546696 |
| Sequence 8748 | S73498 | Genbank | 688010 |
| Sequence 8749 | S74678 | Genbank | 241477 |
| Sequence 8750 | S74799 | Genbank | 797365 |
| Sequence 8751 | S75174 | Genbank | 802120 |
| Sequence 8752 | U82081 | Genbank | 1488412 |
| Sequence 8753 | U00930 | Genbank | 405043 |
| Sequence 8754 | U00947 | Genbank | 405049 |
| Sequence 8755 | U02390 | Genbank | 409928 |
| Sequence 8756 | U02493 | Genbank | 407307 |
| Sequence 8757 | U03886 | Genbank | 458225 |
| Sequence 8758 | U05572 | Genbank | 1419373 |
| Sequence 8759 | U07563 | Genbank | 514264 |
| Sequence 8760 | U07681 | Genbank | 706838 |
| Sequence 8761 | U07810 | Genbank | 469038 |
| Sequence 8762 | U07919 | Genbank | 995897 |
| Sequence 8763 | U08377 | Genbank | 508230 |
| Sequence 8764 | U09220 | Genbank | 799338 |
| Sequence 8765 | U09284 | Genbank | 516011 |
| Sequence 8766 | U09716 | Genbank | 606827 |
| Sequence 8767 | U09813 | Genbank | 1008454 |
| Sequence 8768 | U10248 | Genbank | 984280 |
| Sequence 8769 | U10323 | Genbank | 532312 |
| Sequence 8770 | U10439 | Genbank | 577169 |
| Sequence 8771 | U10691 | Genbank | 533522 |
| Sequence 8772 | U10886 | Genbank | 1685074 |
| Sequence 8773 | U12424 | Genbank | 525319 |
| Sequence 8774 | U12535 | Genbank | 530822 |
| Sequence 8775 | U13022 | Genbank | 537293 |
| Sequence 8776 | U13991 | Genbank | 562076 |
| Sequence 8777 | U14193 | Genbank | 555899 |
| Sequence 8778 | U14966 | Genbank | 550012 |
| Sequence 8779 | U14969 | Genbank | 550018 |
| Sequence 8780 | U14970 | Genbank | 550020 |
| Sequence 8781 | U14971 | Genbank | 550022 |
| Sequence 8782 | U15158 | Genbank | 576622 |
| Sequence 8783 | U16660 | Genbank | 564064 |
| Sequence 8784 | U18121 | Genbank | 915283 |
| Sequence 8785 | U18197 | Genbank | 603073 |
| Sequence 8786 | U18937 | Genbank | 899108 |
| Sequence 8787 | U19145 | Genbank | 914904 |
| Sequence 8788 | U19252 | Genbank | 808035 |
| Sequence 8789 | U20476 | Genbank | 3077819 |
| Sequence 8790 | U21910 | Genbank | 736402 |
| Sequence 8791 | U22055 | Genbank | 799176 |
| Sequence 8792 | U22815 | Genbank | 930340 |
| Sequence 8793 | U23028 | Genbank | 806853 |
| Sequence 8794 | U24169 | Genbank | 1215668 |
| Sequence 8795 | U24223 | Genbank | 1215670 |
| Sequence 8796 | U25816 | Genbank | 837262 |
| Sequence 8797 | U26425 | Genbank | 836664 |
| Sequence 8798 | U27515 | Genbank | 4096274 |
| Sequence 8799 | U28387 | Genbank | 881950 |
| Sequence 8800 | U28686 | Genbank | 881953 |
| Sequence 8801 | U28727 | Genbank | 1142969 |
| Sequence 8802 | U28811 | Genbank | 1373018 |
| Sequence 8803 | U30255 | Genbank | 984324 |
| Sequence 8804 | U32944 | Genbank | 1209060 |
| Sequence 8805 | U32989 | Genbank | 993045 |
| Sequence 8806 | U33460 | Genbank | 4096590 |
| Sequence 8807 | U33760 | Genbank | 995823 |
| Sequence 8808 | U33818 | Genbank | 1163176 |
| Sequence 8809 | U34038 | Genbank | 1041728 |
| Sequence 8810 | U34877 | Genbank | 1143231 |
| Sequence 8811 | U34994 | Genbank | 6525311 |
| Sequence 8812 | U35116 | Genbank | 1008541 |
| Sequence 8813 | U36188 | Genbank | 1244507 |
| Sequence 8814 | U36764 | Genbank | 1036804 |
| Sequence 8815 | U37426 | Genbank | 1171152 |
| Sequence 8816 | U37558 | Genbank | 1017812 |
| Sequence 8817 | U38784 | Genbank | 1574947 |
| Sequence 8818 | U38894 | Genbank | 1589739 |
| Sequence 8819 | U38945 | Genbank | 1353569 |
| Sequence 8820 | U39400 | Genbank | 1234796 |
| Sequence 8821 | U39945 | Genbank | 1209686 |
| Sequence 8822 | U40152 | Genbank | 1113100 |
| Sequence 8823 | U40989 | Genbank | 1125005 |
| Sequence 8824 | U41371 | Genbank | 1173904 |
| Sequence 8825 | U41387 | Genbank | 1230563 |
| Sequence 8826 | U41806 | Genbank | 1145798 |
| Sequence 8827 | U46691 | Genbank | 1401077 |
| Sequence 8828 | U47742 | Genbank | 1517913 |
| Sequence 8829 | U49436 | Genbank | 1229139 |
| Sequence 8830 | U50079 | Genbank | 1277083 |
| Sequence 8831 | U51587 | Genbank | 1669823 |
| Sequence 8832 | U51625 | Genbank | 1695798 |
| Sequence 8833 | U51678 | Genbank | 1915966 |
| Sequence 8834 | U53209 | Genbank | 1256836 |
| Sequence 8835 | U53346 | Genbank | 1545981 |
| Sequence 8836 | U53530 | Genbank | 1314642 |
| Sequence 8837 | U54559 | Genbank | 2351379 |
| Sequence 8838 | U57052 | Genbank | 1519039 |
| Sequence 8839 | U58766 | Genbank | 1381178 |
| Sequence 8840 | U59435 | Genbank | 2697004 |
| Sequence 8841 | U61262 | Genbank | 1621606 |
| Sequence 8842 | U61397 | Genbank | 1518693 |
| Sequence 8843 | U61734 | Genbank | 1407825 |
| Sequence 8844 | U61843 | Genbank | 3650451 |
| Sequence 8845 | U62317 | Genbank | 6862558 |
| Sequence 8846 | U62583 | Genbank | 1778050 |
| Sequence 8847 | U63296 | Genbank | 2047312 |
| Sequence 8848 | U63743 | Genbank | 1695881 |
| Sequence 8849 | U64791 | Genbank | 1498734 |
| Sequence 8850 | U65676 | Genbank | 1654350 |
| Sequence 8851 | U65928 | Genbank | 1549382 |
| Sequence 8852 | U66618 | Genbank | 1549244 |
| Sequence 8853 | U67058 | Genbank | 4097702 |
| Sequence 8854 | U68723 | Genbank | 2114391 |
| Sequence 8855 | U70063 | Genbank | 1743866 |
| Sequence 8856 | U70312 | Genbank | 2865218 |
| Sequence 8857 | U70451 | Genbank | 1763090 |
| Sequence 8858 | U72069 | Genbank | 1657775 |
| Sequence 8859 | U72398 | Genbank | 1622940 |
| Sequence 8860 | U72515 | Genbank | 1673519 |
| Sequence 8861 | U73167 | Genbank | 2880032 |
| Sequence 8862 | U73338 | Genbank | 1763268 |
| Sequence 8863 | U74612 | Genbank | 1842252 |
| Sequence 8864 | U74613 | Genbank | 1842254 |
| Sequence 8865 | U75329 | Genbank | 2507612 |
| Sequence 8866 | U75330 | Genbank | 2507614 |
| Sequence 8867 | U76638 | Genbank | 1710174 |
| Sequence 8868 | U76764 | Genbank | 1685050 |
| Sequence 8869 | U77456 | Genbank | 1679778 |
| Sequence 8870 | U79241 | Genbank | 1710187 |
| Sequence 8871 | U79302 | Genbank | 1710287 |
| Sequence 8872 | U79458 | Genbank | 4205085 |
| Sequence 8873 | U80040 | Genbank | 1718501 |
| Sequence 8874 | U80735 | Genbank | 2565045 |
| Sequence 8875 | U80811 | Genbank | 1857424 |
| Sequence 8876 | U81002 | Genbank | 4580010 |
| Sequence 8877 | U81599 | Genbank | 1764089 |
| Sequence 8878 | U82226 | Genbank | 1848270 |
| Sequence 8879 | U82535 | Genbank | 2149155 |
| Sequence 8880 | U83410 | Genbank | 1923242 |
| Sequence 8881 | U85658 | Genbank | 2058552 |
| Sequence 8882 | U89278 | Genbank | 1877500 |
| Sequence 8883 | U89505 | Genbank | 2078528 |
| Sequence 8884 | U89995 | Genbank | 2078532 |
| Sequence 8885 | U90339 | Genbank | 1906010 |
| Sequence 8886 | U94747 | Genbank | 2290529 |
| Sequence 8887 | U94788 | Genbank | 3041866 |
| Sequence 8888 | U96876 | Genbank | 2358268 |
| Sequence 8889 | U97156 | Genbank | 2459796 |
| Sequence 8890 | U97519 | Genbank | 2213812 |
| Sequence 8891 | V00572 | Genbank | 35434 |
| Sequence 8892 | X01060 | Genbank | 37432 |
| Sequence 8893 | X02152 | Genbank | 34312 |
| Sequence 8894 | X03363 | Genbank | 31197 |
| Sequence 8895 | X03473 | Genbank | 32106 |
| Sequence 8896 | X04408 | Genbank | 31914 |
| Sequence 8897 | X04526 | Genbank | 31667 |
| Sequence 8898 | X04741 | Genbank | 35439 |
| Sequence 8899 | X05332 | Genbank | 35740 |
| Sequence 8900 | X06272 | Genbank | 30865 |
| Sequence 8901 | X06409 | Genbank | 30204 |
| Sequence 8902 | X06562 | Genbank | 31737 |
| Sequence 8903 | X06747 | Genbank | 36101 |
| Sequence 8904 | X06994 | Genbank | 30302 |
| Sequence 8905 | X12433 | Genbank | 32451 |
| Sequence 8906 | X12597 | Genbank | 32326 |
| Sequence 8907 | X12660 | Genbank | 36654 |
| Sequence 8908 | X13294 | Genbank | 28685 |
| Sequence 8909 | X13916 | Genbank | 34338 |
| Sequence 8910 | X13939 | Genbank | 34237 |
| Sequence 8911 | X15488 | Genbank | 30933 |
| Sequence 8912 | X15729 | Genbank | 38317 |
| Sequence 8913 | X15880 | Genbank | 30029 |
| Sequence 8914 | X16064 | Genbank | 37495 |
| Sequence 8915 | X16135 | Genbank | 32355 |
| Sequence 8916 | X16396 | Genbank | 35070 |
| Sequence 8917 | X17567 | Genbank | 36512 |
| Sequence 8918 | X17620 | Genbank | 35067 |
| Sequence 8919 | X51466 | Genbank | 31105 |
| Sequence 8920 | X52519 | Genbank | 36694 |
| Sequence 8921 | X52851 | Genbank | 30167 |
| Sequence 8922 | X52955 | Genbank | 29627 |
| Sequence 8923 | X53280 | Genbank | 29504 |
| Sequence 8924 | X56998 | Genbank | 37564 |
| Sequence 8925 | X57129 | Genbank | 31967 |
| Sequence 8926 | X57206 | Genbank | 33990 |
| Sequence 8927 | X59618 | Genbank | 36154 |
| Sequence 8928 | X61123 | Genbank | 29508 |
| Sequence 8929 | X64123 | Genbank | 35819 |
| Sequence 8930 | X64229 | Genbank | 30502 |
| Sequence 8931 | X64707 | Genbank | 29382 |
| Sequence 8932 | X64875 | Genbank | 398163 |
| Sequence 8933 | X66364 | Genbank | 36620 |
| Sequence 8934 | X67155 | Genbank | 6723674 |
| Sequence 8935 | X67334 | Genbank | 468703 |
| Sequence 8936 | X68836 | Genbank | 36326 |
| Sequence 8937 | X70940 | Genbank | 38455 |
| Sequence 8938 | X71428 | Genbank | 393415 |
| Sequence 8939 | X71810 | Genbank | 297905 |
| Sequence 8940 | X74070 | Genbank | 395086 |
| Sequence 8941 | X74794 | Genbank | 683749 |
| Sequence 8942 | X76057 | Genbank | 416016 |
| Sequence 8943 | X78136 | Genbank | 460772 |
| Sequence 8944 | X78706 | Genbank | 471311 |
| Sequence 8945 | X80197 | Genbank | 951271 |
| Sequence 8946 | X80230 | Genbank | 599828 |
| Sequence 8947 | X80910 | Genbank | 531475 |
| Sequence 8948 | X81197 | Genbank | 773572 |
| Sequence 8949 | X81889 | Genbank | 1702923 |
| Sequence 8950 | X82321 | Genbank | 1617117 |
| Sequence 8951 | X85137 | Genbank | 1155083 |
| Sequence 8952 | X86012 | Genbank | 976082 |
| Sequence 8953 | X92814 | Genbank | 1054751 |
| Sequence 8954 | X94333 | Genbank | 1518268 |
| Sequence 8955 | X94754 | Genbank | 1702931 |
| Sequence 8956 | X95762 | Genbank | 2584786 |
| Sequence 8957 | X96698 | Genbank | 1418766 |
| Sequence 8958 | X97064 | Genbank | 1296663 |
| Sequence 8959 | X97124 | Genbank | 1518264 |
| Sequence 8960 | X97335 | Genbank | 1507823 |
| Sequence 8961 | X97674 | Genbank | 1877214 |
| Sequence 8962 | X97795 | Genbank | 1495482 |
| Sequence 8963 | X98054 | Genbank | 1359754 |
| Sequence 8964 | X98261 | Genbank | 1770459 |
| Sequence 8965 | X98507 | Genbank | 1926310 |
| Sequence 8966 | X99141 | Genbank | 1903215 |
| Sequence 8967 | X99802 | Genbank | 2769561 |
| Sequence 8968 | Y00052 | Genbank | 30308 |
| Sequence 8969 | Y00281 | Genbank | 36052 |
| Sequence 8970 | Y00282 | Genbank | 36048 |
| Sequence 8971 | Y00815 | Genbank | 34266 |
| Sequence 8972 | Y00978 | Genbank | 35359 |
| Sequence 8973 | Y08134 | Genbank | 1552274 |
| Sequence 8974 | Y09862 | Genbank | 1945760 |
| Sequence 8975 | Y10275 | Genbank | 1890330 |
| Sequence 8976 | Y12478 | Genbank | 1946204 |
| Sequence 8977 | Y13621 | Genbank | 2190399 |
| Sequence 8978 | Y13936 | Genbank | 2315201 |
| Sequence 8979 | Y14486 | Genbank | 5830437 |
| Sequence 8980 | Y15286 | Genbank | 2584788 |
| Sequence 8981 | Y18004 | Genbank | 4490941 |
| Sequence 8982 | Z11583 | Genbank | 35118 |
| Sequence 8983 | Z14244 | Genbank | 30150 |
| Sequence 8984 | Z18956 | Genbank | 36726 |
| Sequence 8985 | Z26649 | Genbank | 510116 |
| Sequence 8986 | Z37166 | Genbank | 587145 |
| Sequence 8987 | Z46376 | Genbank | 587201 |
| Sequence 8988 | Z50022 | Genbank | 1107702 |
| Sequence 8989 | Z58902 | Genbank | 1030815 |
| Sequence 8990 | Z69709 | Genbank | 1204107 |
| Sequence 8991 | Z69780 | Genbank | 1514560 |
| Sequence 8992 | Z70200 | Genbank | 3255964 |
| Sequence 8993 | Z71183 | Genbank | 6580491 |
| Sequence 8994 | Z72499 | Genbank | 1545951 |
| Sequence 8995 | Z73358 | Genbank | 1322149 |
| Sequence 8996 | Z73639 | Genbank | 2632099 |
| Sequence 8997 | Z83840 | Genbank | 4914518 |
| Sequence 8998 | Z84466 | Genbank | 3319673 |
| Sequence 8999 | Z92543 | Genbank | 3164068 |
| Sequence 9000 | Z92545 | Genbank | 2462414 |
| Sequence 9001 | Z93783 | Genbank | 4581336 |
| Sequence 9002 | Z95114 | Genbank | 5101742 |
| Sequence 9003 | Z97053 | Genbank | 9650676 |
| Sequence 9004 | Z98258 | Genbank | 2462407 |
| Sequence 9005 | Z98752 | Genbank | 9408718 |
| Sequence 9006 | Z99716 | Genbank | 4456457 |
| Sequence 9007 | AA011002 | EST | 1472029 |
| Sequence 9008 | AA024935 | EST | 1489859 |
| Sequence 9009 | AA025057 | EST | 1489962 |
| Sequence 9010 | AA025381 | EST | 1491520 |
| Sequence 9011 | AA027341 | EST | 1492958 |
| Sequence 9012 | AA031969 | EST | 1501922 |
| Sequence 9013 | AA033521 | EST | 1505367 |
| Sequence 9014 | AA033724 | EST | 1505542 |
| Sequence 9015 | AA037091 | EST | 1512209 |
| Sequence 9016 | AA037126 | EST | 1512234 |
| Sequence 9017 | AA037364 | EST | 1512602 |
| Sequence 9018 | AA040182 | EST | 1516598 |
| Sequence 9019 | AA043058 | EST | 1522593 |
| Sequence 9020 | AA044122 | EST | 1521980 |
| Sequence 9021 | AA044586 | EST | 1522977 |
| Sequence 9022 | AA044813 | EST | 1523016 |
| Sequence 9023 | AA047046 | EST | 1524944 |
| Sequence 9024 | AA053156 | EST | 1544427 |
| Sequence 9025 | AA053431 | EST | 1544086 |
| Sequence 9026 | AA053679 | EST | 1544606 |
| Sequence 9027 | AA054463 | EST | 1545388 |
| Sequence 9028 | AA055407 | EST | 1547946 |
| Sequence 9029 | AA055649 | EST | 1547988 |
| Sequence 9030 | AA056099 | EST | 1548436 |
| Sequence 9031 | AA056273 | EST | 1548820 |
| Sequence 9032 | AA056358 | EST | 1548698 |
| Sequence 9033 | AA058929 | EST | 1551784 |
| Sequence 9034 | AA062553 | EST | 1556727 |
| Sequence 9035 | AA065298 | EST | 1929298 |
| Sequence 9036 | AA069599 | EST | 1576958 |
| Sequence 9037 | AA070333 | EST | 1577692 |
| Sequence 9038 | AA071122 | EST | 1578482 |
| Sequence 9039 | AA074907 | EST | 1614776 |
| Sequence 9040 | AA075864 | EST | 1615735 |
| Sequence 9041 | AA075873 | EST | 1615744 |
| Sequence 9042 | AA075984 | EST | 1615853 |
| Sequence 9043 | AA077364 | EST | 1836838 |
| Sequence 9044 | AA081041 | EST | 1622976 |
| Sequence 9045 | AA081608 | EST | 1623666 |
| Sequence 9046 | AA082133 | EST | 1624191 |
| Sequence 9047 | AA082281 | EST | 1624356 |
| Sequence 9048 | AA082487 | EST | 1624560 |
| Sequence 9049 | AA082543 | EST | 1624600 |
| Sequence 9050 | AA082817 | EST | 1624874 |
| Sequence 9051 | AA083182 | EST | 1625258 |
| Sequence 9052 | AA083288 | EST | 1625349 |
| Sequence 9053 | AA083300 | EST | 1625361 |
| Sequence 9054 | AA088420 | EST | 1633967 |
| Sequence 9055 | AA098867 | EST | 1645051 |
| Sequence 9056 | AA099415 | EST | 1645308 |
| Sequence 9057 | AA099496 | EST | 1645449 |
| Sequence 9058 | AA099923 | EST | 1646071 |
| Sequence 9059 | AA099926 | EST | 1646074 |
| Sequence 9060 | AA101077 | EST | 1647597 |
| Sequence 9061 | AA101100 | EST | 1647676 |
| Sequence 9062 | AA101277 | EST | 1648016 |
| Sequence 9063 | AA101440 | EST | 1648254 |
| Sequence 9064 | AA111991 | EST | 1664113 |
| Sequence 9065 | AA112480 | EST | 1665157 |
| Sequence 9066 | AA112633 | EST | 1665334 |
| Sequence 9067 | AA112679 | EST | 1665397 |
| Sequence 9068 | AA115409 | EST | 1670591 |
| Sequence 9069 | AA121091 | EST | 1678635 |
| Sequence 9070 | AA121210 | EST | 1678949 |
| Sequence 9071 | AA126105 | EST | 1685755 |
| Sequence 9072 | AA126762 | EST | 1686262 |
| Sequence 9073 | AA128709 | EST | 1688617 |
| Sequence 9074 | AA129754 | EST | 1690164 |
| Sequence 9075 | AA130211 | EST | 1691215 |
| Sequence 9076 | AA130220 | EST | 1691224 |
| Sequence 9077 | AA130247 | EST | 1691251 |
| Sequence 9078 | AA130520 | EST | 1691803 |
| Sequence 9079 | AA131227 | EST | 1692735 |
| Sequence 9080 | AA131252 | EST | 1692814 |
| Sequence 9081 | AA131474 | EST | 1692962 |
| Sequence 9082 | AA131496 | EST | 1692984 |
| Sequence 9083 | AA132133 | EST | 1693667 |
| Sequence 9084 | AA132163 | EST | 1693672 |
| Sequence 9085 | AA132328 | EST | 1693818 |
| Sequence 9086 | AA132414 | EST | 1693905 |
| Sequence 9087 | AA132885 | EST | 1694372 |
| Sequence 9088 | AA135266 | EST | 1696351 |
| Sequence 9089 | AA135745 | EST | 1696757 |
| Sequence 9090 | AA135830 | EST | 1697090 |
| Sequence 9091 | AA143151 | EST | 1712521 |
| Sequence 9092 | AA143301 | EST | 1712672 |
| Sequence 9093 | AA147806 | EST | 1717195 |
| Sequence 9094 | AA148139 | EST | 1717530 |
| Sequence 9095 | AA149520 | EST | 1720338 |
| Sequence 9096 | AA149637 | EST | 1720438 |
| Sequence 9097 | AA149750 | EST | 1720892 |
| Sequence 9098 | AA150002 | EST | 1721155 |
| Sequence 9099 | AA150076 | EST | 1721298 |
| Sequence 9100 | AA150383 | EST | 1721914 |
| Sequence 9101 | AA151213 | EST | 1719468 |
| Sequence 9102 | AA151345 | EST | 1719890 |
| Sequence 9103 | AA151506 | EST | 1720046 |
| Sequence 9104 | AA152172 | EST | 1721224 |
| Sequence 9105 | AA156215 | EST | 1727895 |
| Sequence 9106 | AA156807 | EST | 1728440 |
| Sequence 9107 | AA157715 | EST | 1729340 |
| Sequence 9108 | AA158196 | EST | 1733007 |
| Sequence 9109 | AA158600 | EST | 1733436 |
| Sequence 9110 | AA160097 | EST | 1734545 |
| Sequence 9111 | AA160948 | EST | 1735663 |
| Sequence 9112 | AA164729 | EST | 1740889 |
| Sequence 9113 | AA165403 | EST | 1741436 |
| Sequence 9114 | AA165418 | EST | 1741554 |
| Sequence 9115 | AA167191 | EST | 1745698 |
| Sequence 9116 | AA167829 | EST | 1744980 |
| Sequence 9117 | AA169752 | EST | 1748137 |
| Sequence 9118 | AA171588 | EST | 1750657 |
| Sequence 9119 | AA174204 | EST | 1754484 |
| Sequence 9120 | AA179201 | EST | 1760816 |
| Sequence 9121 | AA179430 | EST | 1760782 |
| Sequence 9122 | AA179442 | EST | 1760794 |
| Sequence 9123 | AA179690 | EST | 1761923 |
| Sequence 9124 | AA181608 | EST | 1765136 |
| Sequence 9125 | AA181618 | EST | 1765085 |
| Sequence 9126 | AA186685 | EST | 1774906 |
| Sequence 9127 | AA186730 | EST | 1774847 |
| Sequence 9128 | AA187424 | EST | 1773633 |
| Sequence 9129 | AA187428 | EST | 1773747 |
| Sequence 9130 | AA187809 | EST | 1774003 |
| Sequence 9131 | AA188497 | EST | 1775584 |
| Sequence 9132 | AA188499 | EST | 1775586 |
| Sequence 9133 | AA188875 | EST | 1775902 |
| Sequence 9134 | AA190915 | EST | 1779571 |
| Sequence 9135 | AA191160 | EST | 1779854 |
| Sequence 9136 | AA194169 | EST | 1783860 |
| Sequence 9137 | AA194896 | EST | 1784658 |
| Sequence 9138 | AA195259 | EST | 1784959 |
| Sequence 9139 | AA195352 | EST | 1785043 |
| Sequence 9140 | AA195510 | EST | 1785213 |
| Sequence 9141 | AA195535 | EST | 1785248 |
| Sequence 9142 | AA196350 | EST | 1791925 |
| Sequence 9143 | AA197018 | EST | 1792634 |
| Sequence 9144 | AA197063 | EST | 1792629 |
| Sequence 9145 | AA197137 | EST | 1792811 |
| Sequence 9146 | AA203450 | EST | 1799161 |
| Sequence 9147 | AA205846 | EST | 1801217 |
| Sequence 9148 | AA205928 | EST | 1801316 |
| Sequence 9149 | AA206583 | EST | 1801963 |
| Sequence 9150 | AA207229 | EST | 1802920 |
| Sequence 9151 | AA209239 | EST | 1807254 |
| Sequence 9152 | AA210796 | EST | 1809450 |
| Sequence 9153 | AA211864 | EST | 1810491 |
| Sequence 9154 | AA215319 | EST | 1815082 |
| Sequence 9155 | AA215526 | EST | 1815298 |
| Sequence 9156 | AA215534 | EST | 1815288 |
| Sequence 9157 | AA216769 | EST | 1817460 |
| Sequence 9158 | AA218567 | EST | 1832642 |
| Sequence 9159 | AA218697 | EST | 1832789 |
| Sequence 9160 | AA223382 | EST | 1843907 |
| Sequence 9161 | AA225008 | EST | 1846317 |
| Sequence 9162 | AA226737 | EST | 1848308 |
| Sequence 9163 | AA227227 | EST | 1848772 |
| Sequence 9164 | AA227608 | EST | 1849152 |
| Sequence 9165 | AA232784 | EST | 1855986 |
| Sequence 9166 | AA233063 | EST | 1856075 |
| Sequence 9167 | AA233297 | EST | 1856290 |
| Sequence 9168 | AA234499 | EST | 1858990 |
| Sequence 9169 | AA234963 | EST | 1858132 |
| Sequence 9170 | AA236353 | EST | 1858403 |
| Sequence 9171 | AA236418 | EST | 1858543 |
| Sequence 9172 | AA243646 | EST | 1874456 |
| Sequence 9173 | AA243672 | EST | 1874655 |
| Sequence 9174 | AA247773 | EST | 1879878 |
| Sequence 9175 | AA252002 | EST | 1886963 |
| Sequence 9176 | AA252445 | EST | 1887416 |
| Sequence 9177 | AA252465 | EST | 1887436 |
| Sequence 9178 | AA252567 | EST | 1887541 |
| Sequence 9179 | AA252657 | EST | 1887622 |
| Sequence 9180 | AA255982 | EST | 1891658 |
| Sequence 9181 | AA257153 | EST | 1891281 |
| Sequence 9182 | AA258425 | EST | 1893549 |
| Sequence 9183 | AA258503 | EST | 1893628 |
| Sequence 9184 | AA259061 | EST | 1894541 |
| Sequence 9185 | AA262003 | EST | 1897984 |
| Sequence 9186 | AA262559 | EST | 1898011 |
| Sequence 9187 | AA278264 | EST | 1920222 |
| Sequence 9188 | AA278642 | EST | 1919962 |
| Sequence 9189 | AA278910 | EST | 1920376 |
| Sequence 9190 | AA279735 | EST | 1921200 |
| Sequence 9191 | AA280710 | EST | 1923603 |
| Sequence 9192 | AA281238 | EST | 1923919 |
| Sequence 9193 | AA282006 | EST | 1924848 |
| Sequence 9194 | AA282870 | EST | 1925786 |
| Sequence 9195 | AA282920 | EST | 1925896 |
| Sequence 9196 | AA284386 | EST | 1928720 |
| Sequence 9197 | AA284870 | EST | 1927464 |
| Sequence 9198 | AA287235 | EST | 1934261 |
| Sequence 9199 | AA287990 | EST | 1933814 |
| Sequence 9200 | AA291446 | EST | 1939425 |
| Sequence 9201 | AA291926 | EST | 1940090 |
| Sequence 9202 | AA293348 | EST | 1940745 |
| Sequence 9203 | AA294883 | EST | 1947228 |
| Sequence 9204 | AA296484 | EST | 1948818 |
| Sequence 9205 | AA297767 | EST | 1950170 |
| Sequence 9206 | AA299346 | EST | 1951678 |
| Sequence 9207 | AA300574 | EST | 1953135 |
| Sequence 9208 | AA300728 | EST | 1953296 |
| Sequence 9209 | AA301138 | EST | 1953491 |
| Sequence 9210 | AA301439 | EST | 1953773 |
| Sequence 9211 | AA302601 | EST | 1955083 |
| Sequence 9212 | AA303657 | EST | 1956009 |
| Sequence 9213 | AA304899 | EST | 1957247 |
| Sequence 9214 | AA304904 | EST | 1957469 |
| Sequence 9215 | AA304981 | EST | 1957328 |
| Sequence 9216 | AA305193 | EST | 1957520 |
| Sequence 9217 | AA305525 | EST | 1957850 |
| Sequence 9218 | AA305568 | EST | 1957915 |
| Sequence 9219 | AA305862 | EST | 1958192 |
| Sequence 9220 | AA305873 | EST | 1958203 |
| Sequence 9221 | AA306226 | EST | 1958554 |
| Sequence 9222 | AA306444 | EST | 1958772 |
| Sequence 9223 | AA306511 | EST | 1958840 |
| Sequence 9224 | AA306594 | EST | 1958923 |
| Sequence 9225 | AA306623 | EST | 1958952 |
| Sequence 9226 | AA306709 | EST | 1959037 |
| Sequence 9227 | AA306734 | EST | 1959083 |
| Sequence 9228 | AA306846 | EST | 1959174 |
| Sequence 9229 | AA306917 | EST | 1959247 |
| Sequence 9230 | AA306961 | EST | 1959291 |
| Sequence 9231 | AA306966 | EST | 1959316 |
| Sequence 9232 | AA307060 | EST | 1959538 |
| Sequence 9233 | AA307269 | EST | 1959597 |
| Sequence 9234 | AA307327 | EST | 1959655 |
| Sequence 9235 | AA307332 | EST | 1959660 |
| Sequence 9236 | AA307779 | EST | 1960177 |
| Sequence 9237 | AA307876 | EST | 1960276 |
| Sequence 9238 | AA307979 | EST | 1960308 |
| Sequence 9239 | AA308390 | EST | 1960719 |
| Sequence 9240 | AA308574 | EST | 1960922 |
| Sequence 9241 | AA309051 | EST | 1961377 |
| Sequence 9242 | AA309832 | EST | 1962325 |
| Sequence 9243 | AA310059 | EST | 1962388 |
| Sequence 9244 | AA310330 | EST | 1962679 |
| Sequence 9245 | AA311461 | EST | 1963787 |
| Sequence 9246 | AA311501 | EST | 1964046 |
| Sequence 9247 | AA311933 | EST | 1964261 |
| Sequence 9248 | AA312489 | EST | 1964818 |
| Sequence 9249 | AA312669 | EST | 1965018 |
| Sequence 9250 | AA312778 | EST | 1965126 |
| Sequence 9251 | AA312886 | EST | 1965276 |
| Sequence 9252 | AA313319 | EST | 1965648 |
| Sequence 9253 | AA313489 | EST | 1965818 |
| Sequence 9254 | AA313896 | EST | 1966225 |
| Sequence 9255 | AA314146 | EST | 1966495 |
| Sequence 9256 | AA314350 | EST | 1966679 |
| Sequence 9257 | AA314550 | EST | 1966899 |
| Sequence 9258 | AA314707 | EST | 1967056 |
| Sequence 9259 | AA314717 | EST | 1967277 |
| Sequence 9260 | AA314882 | EST | 1967452 |
| Sequence 9261 | AA315444 | EST | 1967923 |
| Sequence 9262 | AA315472 | EST | 1967801 |
| Sequence 9263 | AA315499 | EST | 1967828 |
| Sequence 9264 | AA315975 | EST | 1968304 |
| Sequence 9265 | AA316030 | EST | 1968359 |
| Sequence 9266 | AA316470 | EST | 1968799 |
| Sequence 9267 | AA316551 | EST | 1968879 |
| Sequence 9268 | AA316590 | EST | 1968918 |
| Sequence 9269 | AA316620 | EST | 1968948 |
| Sequence 9270 | AA316641 | EST | 1968989 |
| Sequence 9271 | AA316743 | EST | 1969142 |
| Sequence 9272 | AA316864 | EST | 1969192 |
| Sequence 9273 | AA317246 | EST | 1969573 |
| Sequence 9274 | AA318236 | EST | 1970564 |
| Sequence 9275 | AA318266 | EST | 1970594 |
| Sequence 9276 | AA323846 | EST | 1976173 |
| Sequence 9277 | AA325370 | EST | 1977838 |
| Sequence 9278 | AA325474 | EST | 1977717 |
| Sequence 9279 | AA326465 | EST | 1978709 |
| Sequence 9280 | AA326466 | EST | 1978710 |
| Sequence 9281 | AA328435 | EST | 1980679 |
| Sequence 9282 | AA329233 | EST | 1981477 |
| Sequence 9283 | AA330793 | EST | 1983035 |
| Sequence 9284 | AA332288 | EST | 1984532 |
| Sequence 9285 | AA333116 | EST | 1985360 |
| Sequence 9286 | AA335696 | EST | 1987937 |
| Sequence 9287 | AA335862 | EST | 1988104 |
| Sequence 9288 | AA336878 | EST | 1989117 |
| Sequence 9289 | AA337404 | EST | 1989869 |
| Sequence 9290 | AA337798 | EST | 1990057 |
| Sequence 9291 | AA338634 | EST | 1990882 |
| Sequence 9292 | AA339622 | EST | 1991880 |
| Sequence 9293 | AA341987 | EST | 1994222 |
| Sequence 9294 | AA343749 | EST | 1995986 |
| Sequence 9295 | AA349886 | EST | 2002205 |
| Sequence 9296 | AA349983 | EST | 2002303 |
| Sequence 9297 | AA351222 | EST | 2003561 |
| Sequence 9298 | AA352688 | EST | 2005008 |
| Sequence 9299 | AA353179 | EST | 2005499 |
| Sequence 9300 | AA353913 | EST | 2006231 |
| Sequence 9301 | AA355954 | EST | 2008272 |
| Sequence 9302 | AA355956 | EST | 2008274 |
| Sequence 9303 | AA356182 | EST | 2008511 |
| Sequence 9304 | AA356195 | EST | 2008524 |
| Sequence 9305 | AA358978 | EST | 2011296 |
| Sequence 9306 | AA359611 | EST | 2012169 |
| Sequence 9307 | AA359735 | EST | 2012052 |
| Sequence 9308 | AA364033 | EST | 2016351 |
| Sequence 9309 | AA367068 | EST | 2019406 |
| Sequence 9310 | AA368087 | EST | 2020446 |
| Sequence 9311 | AA369721 | EST | 2022039 |
| Sequence 9312 | AA370428 | EST | 2022933 |
| Sequence 9313 | AA371420 | EST | 2023780 |
| Sequence 9314 | AA371957 | EST | 2024275 |
| Sequence 9315 | AA372674 | EST | 2025182 |
| Sequence 9316 | AA375636 | EST | 2027956 |
| Sequence 9317 | AA376361 | EST | 2028679 |
| Sequence 9318 | AA376458 | EST | 2028777 |
| Sequence 9319 | AA378378 | EST | 2030695 |
| Sequence 9320 | AA378849 | EST | 2031168 |
| Sequence 9321 | AA386348 | EST | 2038701 |
| Sequence 9322 | AA397884 | EST | 2050623 |
| Sequence 9323 | AA399142 | EST | 2052896 |
| Sequence 9324 | AA399177 | EST | 2052913 |
| Sequence 9325 | AA401252 | EST | 2055141 |
| Sequence 9326 | AA401261 | EST | 2055168 |
| Sequence 9327 | AA401526 | EST | 2053804 |
| Sequence 9328 | AA402063 | EST | 2056854 |
| Sequence 9329 | AA403047 | EST | 2055609 |
| Sequence 9330 | AA406425 | EST | 2064410 |
| Sequence 9331 | AA412426 | EST | 2071129 |
| Sequence 9332 | AA417300 | EST | 2077408 |
| Sequence 9333 | AA417999 | EST | 2079818 |
| Sequence 9334 | AA418045 | EST | 2079864 |
| Sequence 9335 | AA418096 | EST | 2079897 |
| Sequence 9336 | AA418249 | EST | 2080078 |
| Sequence 9337 | AA421643 | EST | 2100459 |
| Sequence 9338 | AA424317 | EST | 2103287 |
| Sequence 9339 | AA425387 | EST | 2106170 |
| Sequence 9340 | AA425619 | EST | 2106375 |
| Sequence 9341 | AA425658 | EST | 2106433 |
| Sequence 9342 | AA425751 | EST | 2106453 |
| Sequence 9343 | AA425761 | EST | 2106481 |
| Sequence 9344 | AA427920 | EST | 2112054 |
| Sequence 9345 | AA428948 | EST | 2110491 |
| Sequence 9346 | AA429871 | EST | 2113108 |
| Sequence 9347 | AA431577 | EST | 2115285 |
| Sequence 9348 | AA433865 | EST | 2138779 |
| Sequence 9349 | AA436966 | EST | 2141880 |
| Sequence 9350 | AA442297 | EST | 2154175 |
| Sequence 9351 | AA442687 | EST | 2154565 |
| Sequence 9352 | AA446391 | EST | 2159056 |
| Sequence 9353 | AA448292 | EST | 2161962 |
| Sequence 9354 | AA449483 | EST | 2163233 |
| Sequence 9355 | AA449501 | EST | 2163251 |
| Sequence 9356 | AA449646 | EST | 2163396 |
| Sequence 9357 | AA450096 | EST | 2163846 |
| Sequence 9358 | AA450166 | EST | 2163916 |
| Sequence 9359 | AA450276 | EST | 2164026 |
| Sequence 9360 | AA452716 | EST | 2166385 |
| Sequence 9361 | AA453565 | EST | 2167234 |
| Sequence 9362 | AA453899 | EST | 2167568 |
| Sequence 9363 | AA456454 | EST | 2179030 |
| Sequence 9364 | AA457365 | EST | 2180085 |
| Sequence 9365 | AA458613 | EST | 2183520 |
| Sequence 9366 | AA459167 | EST | 2184074 |
| Sequence 9367 | AA461383 | EST | 2186503 |
| Sequence 9368 | AA463367 | EST | 2188251 |
| Sequence 9369 | AA465402 | EST | 2191569 |
| Sequence 9370 | AA469033 | EST | 2195567 |
| Sequence 9371 | AA470067 | EST | 2197376 |
| Sequence 9372 | AA476876 | EST | 2205087 |
| Sequence 9373 | AA478162 | EST | 2206796 |
| Sequence 9374 | AA479499 | EST | 2208055 |
| Sequence 9375 | AA480816 | EST | 2210368 |
| Sequence 9376 | AA481233 | EST | 2210785 |
| Sequence 9377 | AA481838 | EST | 2209516 |
| Sequence 9378 | AA485255 | EST | 2214474 |
| Sequence 9379 | AA485520 | EST | 2214739 |
| Sequence 9380 | AA488447 | EST | 2215878 |
| Sequence 9381 | AA488488 | EST | 2215919 |
| Sequence 9382 | AA488513 | EST | 2215944 |
| Sequence 9383 | AA489117 | EST | 2218719 |
| Sequence 9384 | AA489288 | EST | 2218890 |
| Sequence 9385 | AA490389 | EST | 2219562 |
| Sequence 9386 | AA491213 | EST | 2220386 |
| Sequence 9387 | AA491638 | EST | 2221200 |
| Sequence 9388 | AA494334 | EST | 2224121 |
| Sequence 9389 | AA494355 | EST | 2224142 |
| Sequence 9390 | AA494387 | EST | 2224174 |
| Sequence 9391 | AA495912 | EST | 2229233 |
| Sequence 9392 | AA496486 | EST | 2229807 |
| Sequence 9393 | AA501442 | EST | 2236409 |
| Sequence 9394 | AA504269 | EST | 2240429 |
| Sequence 9395 | AA505816 | EST | 2241953 |
| Sequence 9396 | AA507818 | EST | 2244257 |
| Sequence 9397 | AA508038 | EST | 2244477 |
| Sequence 9398 | AA514302 | EST | 2253810 |
| Sequence 9399 | AA516531 | EST | 2256055 |
| Sequence 9400 | AA522888 | EST | 2263600 |
| Sequence 9401 | AA527374 | EST | 2269443 |
| Sequence 9402 | AA532530 | EST | 2276784 |
| Sequence 9403 | AA532673 | EST | 2276927 |
| Sequence 9404 | AA533360 | EST | 2277456 |
| Sequence 9405 | AA534734 | EST | 2278987 |
| Sequence 9406 | AA535663 | EST | 2279916 |
| Sequence 9407 | AA535845 | EST | 2280098 |
| Sequence 9408 | AA552507 | EST | 2322761 |
| Sequence 9409 | AA558357 | EST | 2328834 |
| Sequence 9410 | AA565548 | EST | 2337187 |
| Sequence 9411 | AA565867 | EST | 2337506 |
| Sequence 9412 | AA568785 | EST | 2341839 |
| Sequence 9413 | AA573427 | EST | 2347955 |
| Sequence 9414 | AA574223 | EST | 2348738 |
| Sequence 9415 | AA576016 | EST | 2350531 |
| Sequence 9416 | AA576889 | EST | 2354363 |
| Sequence 9417 | AA579611 | EST | 2357795 |
| Sequence 9418 | AA581185 | EST | 2358957 |
| Sequence 9419 | AA587264 | EST | 2398078 |
| Sequence 9420 | AA587570 | EST | 2397245 |
| Sequence 9421 | AA594562 | EST | 2409912 |
| Sequence 9422 | AA598697 | EST | 2432369 |
| Sequence 9423 | AA600084 | EST | 2433709 |
| Sequence 9424 | AA600355 | EST | 2433980 |
| Sequence 9425 | AA601289 | EST | 2434914 |
| Sequence 9426 | AA603876 | EST | 2437737 |
| Sequence 9427 | AA604860 | EST | 2445724 |
| Sequence 9428 | AA608613 | EST | 2457041 |
| Sequence 9429 | AA609417 | EST | 2457845 |
| Sequence 9430 | AA612668 | EST | 2463706 |
| Sequence 9431 | AA625213 | EST | 2537598 |
| Sequence 9432 | AA626629 | EST | 2539016 |
| Sequence 9433 | AA627320 | EST | 2540337 |
| Sequence 9434 | AA628491 | EST | 2540878 |
| Sequence 9435 | AA628520 | EST | 2540907 |
| Sequence 9436 | AA631993 | EST | 2555407 |
| Sequence 9437 | AA634958 | EST | 2558172 |
| Sequence 9438 | AA640716 | EST | 2565966 |
| Sequence 9439 | AA641939 | EST | 2567157 |
| Sequence 9440 | AA652388 | EST | 2584040 |
| Sequence 9441 | AA659693 | EST | 2595847 |
| Sequence 9442 | AA662024 | EST | 2616115 |
| Sequence 9443 | AA663649 | EST | 2617640 |
| Sequence 9444 | AA668886 | EST | 2630385 |
| Sequence 9445 | AA679400 | EST | 2659922 |
| Sequence 9446 | AA679621 | EST | 2660143 |
| Sequence 9447 | AA687221 | EST | 2675412 |
| Sequence 9448 | AA687495 | EST | 2675686 |
| Sequence 9449 | AA693932 | EST | 2694870 |
| Sequence 9450 | AA694188 | EST | 2695126 |
| Sequence 9451 | AA700208 | EST | 2703171 |
| Sequence 9452 | AA701138 | EST | 2704303 |
| Sequence 9453 | AA704533 | EST | 2714451 |
| Sequence 9454 | AA705937 | EST | 2715855 |
| Sequence 9455 | AA709002 | EST | 2718920 |
| Sequence 9456 | AA716423 | EST | 2728697 |
| Sequence 9457 | AA722455 | EST | 2740162 |
| Sequence 9458 | AA722786 | EST | 2740493 |
| Sequence 9459 | AA730400 | EST | 2751604 |
| Sequence 9460 | AA731394 | EST | 2753550 |
| Sequence 9461 | AA742260 | EST | 2784260 |
| Sequence 9462 | AA746310 | EST | 2786296 |
| Sequence 9463 | AA746748 | EST | 2786734 |
| Sequence 9464 | AA747197 | EST | 2787155 |
| Sequence 9465 | AA749387 | EST | 2789345 |
| Sequence 9466 | AA757433 | EST | 2805296 |
| Sequence 9467 | AA761101 | EST | 2810031 |
| Sequence 9468 | AA765907 | EST | 2817145 |
| Sequence 9469 | AA769588 | EST | 2820826 |
| Sequence 9470 | AA772570 | EST | 2824353 |
| Sequence 9471 | AA774779 | EST | 2834113 |
| Sequence 9472 | AA775625 | EST | 2834959 |
| Sequence 9473 | AA805474 | EST | 2874224 |
| Sequence 9474 | AA808205 | EST | 2877611 |
| Sequence 9475 | AA808239 | EST | 2877645 |
| Sequence 9476 | AA813991 | EST | 2883587 |
| Sequence 9477 | AA814182 | EST | 2883778 |
| Sequence 9478 | AA814817 | EST | 2884413 |
| Sequence 9479 | AA826906 | EST | 2900903 |
| Sequence 9480 | AA830072 | EST | 2903171 |
| Sequence 9481 | AA831991 | EST | 2905090 |
| Sequence 9482 | AA833749 | EST | 2908517 |
| Sequence 9483 | AA837407 | EST | 2912606 |
| Sequence 9484 | AA838157 | EST | 2913814 |
| Sequence 9485 | AA843207 | EST | 2929725 |
| Sequence 9486 | AA843238 | EST | 2929756 |
| Sequence 9487 | AA843878 | EST | 2930329 |
| Sequence 9488 | AA843908 | EST | 2930359 |
| Sequence 9489 | AA857964 | EST | 2946266 |
| Sequence 9490 | AA864964 | EST | 2959277 |
| Sequence 9491 | AA868452 | EST | 2963897 |
| Sequence 9492 | AA868722 | EST | 2964167 |
| Sequence 9493 | AA872507 | EST | 2968685 |
| Sequence 9494 | AA872913 | EST | 2969035 |
| Sequence 9495 | AA872991 | EST | 2969113 |
| Sequence 9496 | AA877350 | EST | 2986427 |
| Sequence 9497 | AA877789 | EST | 2986754 |
| Sequence 9498 | AA902103 | EST | 3037293 |
| Sequence 9499 | AA902974 | EST | 3038097 |
| Sequence 9500 | AA903253 | EST | 3038376 |
| Sequence 9501 | AA905330 | EST | 3040453 |
| Sequence 9502 | AA906868 | EST | 3042112 |
| Sequence 9503 | AA907186 | EST | 3042646 |
| Sequence 9504 | AA908460 | EST | 3047865 |
| Sequence 9505 | AA913203 | EST | 3052595 |
| Sequence 9506 | AA916003 | EST | 3055395 |
| Sequence 9507 | AA927612 | EST | 3077033 |
| Sequence 9508 | AA931566 | EST | 3085952 |
| Sequence 9509 | AA932278 | EST | 3086516 |
| Sequence 9510 | AA968524 | EST | 3143704 |
| Sequence 9511 | AA973790 | EST | 3148970 |
| Sequence 9512 | AA976114 | EST | 3151906 |
| Sequence 9513 | AA976653 | EST | 3154099 |
| Sequence 9514 | AA984122 | EST | 3162647 |
| Sequence 9515 | AA988106 | EST | 3173470 |
| Sequence 9516 | AA988525 | EST | 3174217 |
| Sequence 9517 | AA988576 | EST | 3174268 |
| Sequence 9518 | AA991580 | EST | 3178069 |
| Sequence 9519 | AF001542 | EST | 2529714 |
| Sequence 9520 | AI015791 | EST | 3230127 |
| Sequence 9521 | AI017095 | EST | 3231431 |
| Sequence 9522 | AI017902 | EST | 3232238 |
| Sequence 9523 | AI018625 | EST | 3233144 |
| Sequence 9524 | AI022209 | EST | 3239562 |
| Sequence 9525 | AI027035 | EST | 3244551 |
| Sequence 9526 | AI033991 | EST | 3254944 |
| Sequence 9527 | AI034371 | EST | 3255324 |
| Sequence 9528 | AI040602 | EST | 3279796 |
| Sequence 9529 | AI051466 | EST | 3307000 |
| Sequence 9530 | AI052000 | EST | 3307991 |
| Sequence 9531 | AI056337 | EST | 3330203 |
| Sequence 9532 | AI056402 | EST | 3330268 |
| Sequence 9533 | AI061175 | EST | 3336543 |
| Sequence 9534 | AI064910 | EST | 6359182 |
| Sequence 9535 | AI066583 | EST | 3367285 |
| Sequence 9536 | AI076630 | EST | 3405808 |
| Sequence 9537 | AI076953 | EST | 3404782 |
| Sequence 9538 | AI078217 | EST | 3412625 |
| Sequence 9539 | AI079510 | EST | 3415761 |
| Sequence 9540 | AI080497 | EST | 3416748 |
| Sequence 9541 | AI081780 | EST | 3418572 |
| Sequence 9542 | AI084794 | EST | 3423217 |
| Sequence 9543 | AI084916 | EST | 3423339 |
| Sequence 9544 | AI085616 | EST | 3424039 |
| Sequence 9545 | AI088897 | EST | 3427956 |
| Sequence 9546 | AI090197 | EST | 3429256 |
| Sequence 9547 | AI091173 | EST | 3430232 |
| Sequence 9548 | AI094514 | EST | 3433490 |
| Sequence 9549 | AI095450 | EST | 3434426 |
| Sequence 9550 | AI114692 | EST | 6360037 |
| Sequence 9551 | AI123418 | EST | 3539184 |
| Sequence 9552 | AI124792 | EST | 3593306 |
| Sequence 9553 | AI126005 | EST | 3594519 |
| Sequence 9554 | AI129250 | EST | 3597764 |
| Sequence 9555 | AI133074 | EST | 6360390 |
| Sequence 9556 | AI133088 | EST | 6360404 |
| Sequence 9557 | AI133274 | EST | 6360590 |
| Sequence 9558 | AI133426 | EST | 6360742 |
| Sequence 9559 | AI139764 | EST | 3645736 |
| Sequence 9560 | AI142414 | EST | 3658773 |
| Sequence 9561 | AI146833 | EST | 3674515 |
| Sequence 9562 | AI148106 | EST | 3675788 |
| Sequence 9563 | AI150430 | EST | 3678899 |
| Sequence 9564 | AI151146 | EST | 3679615 |
| Sequence 9565 | AI159774 | EST | 3693133 |
| Sequence 9566 | AI161072 | EST | 3694377 |
| Sequence 9567 | AI168316 | EST | 3701486 |
| Sequence 9568 | AI168348 | EST | 3701518 |
| Sequence 9569 | AI186453 | EST | 3737091 |
| Sequence 9570 | AI187157 | EST | 3737795 |
| Sequence 9571 | AI189475 | EST | 3740684 |
| Sequence 9572 | AI193372 | EST | 3744581 |
| Sequence 9573 | AI198218 | EST | 3750824 |
| Sequence 9574 | AI199191 | EST | 3751797 |
| Sequence 9575 | AI199194 | EST | 3751800 |
| Sequence 9576 | AI202137 | EST | 3754743 |
| Sequence 9577 | AI203214 | EST | 3755820 |
| Sequence 9578 | AI207471 | EST | 6361479 |
| Sequence 9579 | AI207868 | EST | 3769810 |
| Sequence 9580 | AI214048 | EST | 3777649 |
| Sequence 9581 | AI214453 | EST | 3778054 |
| Sequence 9582 | AI216098 | EST | 3785139 |
| Sequence 9583 | AI217246 | EST | 3797061 |
| Sequence 9584 | AI244268 | EST | 3839665 |
| Sequence 9585 | AI248386 | EST | 3843783 |
| Sequence 9586 | AI249242 | EST | 3845771 |
| Sequence 9587 | AI251739 | EST | 3848268 |
| Sequence 9588 | AI261980 | EST | 3870183 |
| Sequence 9589 | AI262251 | EST | 3870454 |
| Sequence 9590 | AI267162 | EST | 3886329 |
| Sequence 9591 | AI267848 | EST | 3887015 |
| Sequence 9592 | AI268335 | EST | 3887502 |
| Sequence 9593 | AI268362 | EST | 3887529 |
| Sequence 9594 | AI270685 | EST | 3889852 |
| Sequence 9595 | AI275466 | EST | 3897740 |
| Sequence 9596 | AI276933 | EST | 3899201 |
| Sequence 9597 | AI277443 | EST | 3899711 |
| Sequence 9598 | AI278328 | EST | 3900596 |
| Sequence 9599 | AI279625 | EST | 3917859 |
| Sequence 9600 | AI280561 | EST | 3918794 |
| Sequence 9601 | AI281394 | EST | 3919627 |
| Sequence 9602 | AI283373 | EST | 3921606 |
| Sequence 9603 | AI283578 | EST | 3921811 |
| Sequence 9604 | AI284482 | EST | 3922715 |
| Sequence 9605 | AI290052 | EST | 3931718 |
| Sequence 9606 | AI290111 | EST | 3931777 |
| Sequence 9607 | AI290154 | EST | 3931820 |
| Sequence 9608 | AI291087 | EST | 3933861 |
| Sequence 9609 | AI292094 | EST | 3934868 |
| Sequence 9610 | AI298974 | EST | 3958628 |
| Sequence 9611 | AI333116 | EST | 4069675 |
| Sequence 9612 | AI334177 | EST | 4070736 |
| Sequence 9613 | AI334889 | EST | 4071816 |
| Sequence 9614 | AI337358 | EST | 4074285 |
| Sequence 9615 | AI339284 | EST | 4076211 |
| Sequence 9616 | AI341138 | EST | 4078065 |
| Sequence 9617 | AI341291 | EST | 4078218 |
| Sequence 9618 | AI342090 | EST | 4079017 |
| Sequence 9619 | AI343404 | EST | 4080610 |
| Sequence 9620 | AI344189 | EST | 4081395 |
| Sequence 9621 | AI348588 | EST | 4085794 |
| Sequence 9622 | AI349337 | EST | 4086543 |
| Sequence 9623 | AI349957 | EST | 4087163 |
| Sequence 9624 | AI350524 | EST | 4087730 |
| Sequence 9625 | AI352184 | EST | 4089390 |
| Sequence 9626 | AI355277 | EST | 4095430 |
| Sequence 9627 | AI356461 | EST | 4108082 |
| Sequence 9628 | AI357566 | EST | 4109187 |
| Sequence 9629 | AI357582 | EST | 4109203 |
| Sequence 9630 | AI360681 | EST | 4112302 |
| Sequence 9631 | AI361796 | EST | 4113417 |
| Sequence 9632 | AI373828 | EST | 4153694 |
| Sequence 9633 | AI378900 | EST | 4188753 |
| Sequence 9634 | AI379649 | EST | 4189502 |
| Sequence 9635 | AI381737 | EST | 4194518 |
| Sequence 9636 | AI381972 | EST | 4194753 |
| Sequence 9637 | AI393269 | EST | 4222816 |
| Sequence 9638 | AI420586 | EST | 4266517 |
| Sequence 9639 | AI435112 | EST | 4300898 |
| Sequence 9640 | AI439421 | EST | 4304204 |
| Sequence 9641 | AI439588 | EST | 4305374 |
| Sequence 9642 | AI445716 | EST | 4290157 |
| Sequence 9643 | AI453049 | EST | 4307708 |
| Sequence 9644 | AI457432 | EST | 4310301 |
| Sequence 9645 | AI458254 | EST | 4310833 |
| Sequence 9646 | AI458599 | EST | 4311178 |
| Sequence 9647 | AI458758 | EST | 4311337 |
| Sequence 9648 | AI460220 | EST | 4313101 |
| Sequence 9649 | AI468419 | EST | 4330509 |
| Sequence 9650 | AI472084 | EST | 4334174 |
| Sequence 9651 | AI472566 | EST | 4325611 |
| Sequence 9652 | AI479075 | EST | 4372243 |
| Sequence 9653 | AI480254 | EST | 4373422 |
| Sequence 9654 | AI492032 | EST | 4393035 |
| Sequence 9655 | AI494037 | EST | 4395040 |
| Sequence 9656 | AI525541 | EST | 4439676 |
| Sequence 9657 | AI525654 | EST | 4439789 |
| Sequence 9658 | AI538842 | EST | 4452977 |
| Sequence 9659 | AI557226 | EST | 4489589 |
| Sequence 9660 | AI560973 | EST | 4511314 |
| Sequence 9661 | AI563921 | EST | 4522378 |
| Sequence 9662 | AI564719 | EST | 4523176 |
| Sequence 9663 | AI570615 | EST | 4533989 |
| Sequence 9664 | AI580252 | EST | 4564628 |
| Sequence 9665 | AI580336 | EST | 4564712 |
| Sequence 9666 | AI581098 | EST | 4565474 |
| Sequence 9667 | AI597918 | EST | 4606977 |
| Sequence 9668 | AI608796 | EST | 4617963 |
| Sequence 9669 | AI609087 | EST | 4618254 |
| Sequence 9670 | AI610880 | EST | 4620047 |
| Sequence 9671 | AI623984 | EST | 4648915 |
| Sequence 9672 | AI624751 | EST | 4649682 |
| Sequence 9673 | AI628354 | EST | 4665154 |
| Sequence 9674 | AI628417 | EST | 4665217 |
| Sequence 9675 | AI632470 | EST | 4683800 |
| Sequence 9676 | AI633000 | EST | 4684330 |
| Sequence 9677 | AI637826 | EST | 4690060 |
| Sequence 9678 | AI638561 | EST | 4690795 |
| Sequence 9679 | AI640607 | EST | 4703716 |
| Sequence 9680 | AI650583 | EST | 4734562 |
| Sequence 9681 | AI651877 | EST | 4735856 |
| Sequence 9682 | AI672492 | EST | 4852223 |
| Sequence 9683 | AI684595 | EST | 4895889 |
| Sequence 9684 | AI685930 | EST | 4897224 |
| Sequence 9685 | AI690089 | EST | 4901383 |
| Sequence 9686 | AI692658 | EST | 4969998 |
| Sequence 9687 | AI694944 | EST | 4982844 |
| Sequence 9688 | AI697185 | EST | 4985085 |
| Sequence 9689 | AI720354 | EST | 5037610 |
| Sequence 9690 | AI720743 | EST | 5037999 |
| Sequence 9691 | AI738670 | EST | 5100651 |
| Sequence 9692 | AI743182 | EST | 5111470 |
| Sequence 9693 | AI743921 | EST | 5112209 |
| Sequence 9694 | AI750105 | EST | 5128369 |
| Sequence 9695 | AI751208 | EST | 5129472 |
| Sequence 9696 | AI751459 | EST | 5129723 |
| Sequence 9697 | AI751487 | EST | 5129751 |
| Sequence 9698 | AI752264 | EST | 5130528 |
| Sequence 9699 | AI752414 | EST | 5130678 |
| Sequence 9700 | AI752425 | EST | 5130689 |
| Sequence 9701 | AI760045 | EST | 5175712 |
| Sequence 9702 | AI761315 | EST | 5176982 |
| Sequence 9703 | AI765501 | EST | 5232010 |
| Sequence 9704 | AI766305 | EST | 5232814 |
| Sequence 9705 | AI767578 | EST | 5234087 |
| Sequence 9706 | AI767726 | EST | 5234235 |
| Sequence 9707 | AI792015 | EST | 5339731 |
| Sequence 9708 | AI795827 | EST | 5361287 |
| Sequence 9709 | AI797228 | EST | 5362700 |
| Sequence 9710 | AI800215 | EST | 5365687 |
| Sequence 9711 | AI806017 | EST | 5392583 |
| Sequence 9712 | AI807798 | EST | 5394364 |
| Sequence 9713 | AI808865 | EST | 5395431 |
| Sequence 9714 | AI810669 | EST | 5397235 |
| Sequence 9715 | AI814463 | EST | 5425678 |
| Sequence 9716 | AI814513 | EST | 5425728 |
| Sequence 9717 | AI815492 | EST | 5431038 |
| Sequence 9718 | AI816438 | EST | 5431984 |
| Sequence 9719 | AI818683 | EST | 5437762 |
| Sequence 9720 | AI819405 | EST | 5438484 |
| Sequence 9721 | AI828075 | EST | 5448746 |
| Sequence 9722 | AI863382 | EST | 5527489 |
| Sequence 9723 | AI873001 | EST | 5547050 |
| Sequence 9724 | AI873501 | EST | 5547550 |
| Sequence 9725 | AI874326 | EST | 5548375 |
| Sequence 9726 | AI884491 | EST | 5589655 |
| Sequence 9727 | AI884783 | EST | 5589947 |
| Sequence 9728 | AI885021 | EST | 5590185 |
| Sequence 9729 | AI887758 | EST | 5592922 |
| Sequence 9730 | AI888327 | EST | 5593577 |
| Sequence 9731 | AI888872 | EST | 5594036 |
| Sequence 9732 | AI903705 | EST | 6494092 |
| Sequence 9733 | AI904447 | EST | 6494834 |
| Sequence 9734 | AI906328 | EST | 6496715 |
| Sequence 9735 | AI910090 | EST | 6500770 |
| Sequence 9736 | AI914133 | EST | 5633988 |
| Sequence 9737 | AI918605 | EST | 5638460 |
| Sequence 9738 | AI924534 | EST | 5660498 |
| Sequence 9739 | AI927755 | EST | 5663719 |
| Sequence 9740 | AI929696 | EST | 5665660 |
| Sequence 9741 | AI936166 | EST | 5675036 |
| Sequence 9742 | AI950991 | EST | 5743301 |
| Sequence 9743 | AI970095 | EST | 5766921 |
| Sequence 9744 | AI972423 | EST | 5769339 |
| Sequence 9745 | AI982834 | EST | 5810053 |
| Sequence 9746 | AI982890 | EST | 5810109 |
| Sequence 9747 | AL035972 | EST | 5405602 |
| Sequence 9748 | AL036220 | EST | 5405817 |
| Sequence 9749 | AL036415 | EST | 5405971 |
| Sequence 9750 | AL037386 | EST | 5406786 |
| Sequence 9751 | AL037968 | EST | 5928298 |
| Sequence 9752 | AL039569 | EST | 5408608 |
| Sequence 9753 | AL039961 | EST | 5408942 |
| Sequence 9754 | AL040667 | EST | 5409613 |
| Sequence 9755 | AL040692 | EST | 5409638 |
| Sequence 9756 | AL041673 | EST | 5421022 |
| Sequence 9757 | AL042613 | EST | 5935505 |
| Sequence 9758 | AL042680 | EST | 5422129 |
| Sequence 9759 | AL042725 | EST | 5422174 |
| Sequence 9760 | AL042986 | EST | 5935655 |
| Sequence 9761 | AL043210 | EST | 5935803 |
| Sequence 9762 | AL043542 | EST | 5422929 |
| Sequence 9763 | AL043672 | EST | 5423059 |
| Sequence 9764 | AL045757 | EST | 5936079 |
| Sequence 9765 | AL045793 | EST | 5433905 |
| Sequence 9766 | AL045936 | EST | 5434039 |
| Sequence 9767 | AL045937 | EST | 5936123 |
| Sequence 9768 | AL045976 | EST | 5434076 |
| Sequence 9769 | AL046015 | EST | 5434109 |
| Sequence 9770 | AL047001 | EST | 5435057 |
| Sequence 9771 | AL047332 | EST | 4727279 |
| Sequence 9772 | AL047890 | EST | 4728078 |
| Sequence 9773 | AL048554 | EST | 5936565 |
| Sequence 9774 | AL110421 | EST | 5866029 |
| Sequence 9775 | AL118756 | EST | 5924655 |
| Sequence 9776 | AL118845 | EST | 5924744 |
| Sequence 9777 | AL119809 | EST | 5925708 |
| Sequence 9778 | AL120711 | EST | 5926610 |
| Sequence 9779 | AL120865 | EST | 5926866 |
| Sequence 9780 | AL120911 | EST | 5926912 |
| Sequence 9781 | AL121473 | EST | 5927474 |
| Sequence 9782 | AL121558 | EST | 5927559 |
| Sequence 9783 | AL133779 | EST | 6601967 |
| Sequence 9784 | AL133900 | EST | 6602087 |
| Sequence 9785 | AL134724 | EST | 6602911 |
| Sequence 9786 | AL134795 | EST | 6602982 |
| Sequence 9787 | AL134938 | EST | 6603125 |
| Sequence 9788 | AL135007 | EST | 6603194 |
| Sequence 9789 | AL135023 | EST | 6603210 |
| Sequence 9790 | AL135290 | EST | 6603477 |
| Sequence 9791 | AL135343 | EST | 6603530 |
| Sequence 9792 | AL135362 | EST | 6603549 |
| Sequence 9793 | AL135502 | EST | 6603689 |
| Sequence 9794 | AL135600 | EST | 6603787 |
| Sequence 9795 | AL138042 | EST | 6854722 |
| Sequence 9796 | AW002442 | EST | 5849358 |
| Sequence 9797 | AW014499 | EST | 5863256 |
| Sequence 9798 | AW014727 | EST | 5863484 |
| Sequence 9799 | AW029611 | EST | 5888367 |
| Sequence 9800 | AW029642 | EST | 5888398 |
| Sequence 9801 | AW043897 | EST | 5904426 |
| Sequence 9802 | AW062837 | EST | 6014222 |
| Sequence 9803 | AW068104 | EST | 6023102 |
| Sequence 9804 | AW070431 | EST | 6025429 |
| Sequence 9805 | AW070690 | EST | 6025688 |
| Sequence 9806 | AW080672 | EST | 6035905 |
| Sequence 9807 | AW084080 | EST | 6039232 |
| Sequence 9808 | AW118447 | EST | 6087031 |
| Sequence 9809 | AW148635 | EST | 6196531 |
| Sequence 9810 | AW149925 | EST | 6197821 |
| Sequence 9811 | AW157231 | EST | 6228632 |
| Sequence 9812 | AW160468 | EST | 6299501 |
| Sequence 9813 | AW160833 | EST | 6299866 |
| Sequence 9814 | AW161807 | EST | 6300840 |
| Sequence 9815 | AW161978 | EST | 6301011 |
| Sequence 9816 | AW162587 | EST | 6301620 |
| Sequence 9817 | AW162761 | EST | 6301794 |
| Sequence 9818 | AW162774 | EST | 6301807 |
| Sequence 9819 | AW163485 | EST | 6302518 |
| Sequence 9820 | AW163783 | EST | 6302816 |
| Sequence 9821 | AW175627 | EST | 6441768 |
| Sequence 9822 | AW177931 | EST | 6443968 |
| Sequence 9823 | AW178072 | EST | 6444109 |
| Sequence 9824 | AW194013 | EST | 6472742 |
| Sequence 9825 | AW237543 | EST | 6569932 |
| Sequence 9826 | AW239041 | EST | 6571431 |
| Sequence 9827 | AW239281 | EST | 6571671 |
| Sequence 9828 | AW245631 | EST | 6588624 |
| Sequence 9829 | AW245889 | EST | 6588882 |
| Sequence 9830 | AW245906 | EST | 6588899 |
| Sequence 9831 | AW245923 | EST | 6588916 |
| Sequence 9832 | AW246049 | EST | 6589042 |
| Sequence 9833 | AW246074 | EST | 6589067 |
| Sequence 9834 | AW247459 | EST | 6590452 |
| Sequence 9835 | AW247494 | EST | 6590487 |
| Sequence 9836 | AW249281 | EST | 6592274 |
| Sequence 9837 | AW250125 | EST | 6593118 |
| Sequence 9838 | AW250126 | EST | 6593119 |
| Sequence 9839 | AW263239 | EST | 6640055 |
| Sequence 9840 | AW270779 | EST | 6657809 |
| Sequence 9841 | AW273055 | EST | 6660085 |
| Sequence 9842 | AW274583 | EST | 6661613 |
| Sequence 9843 | AW292896 | EST | 6699532 |
| Sequence 9844 | AW292897 | EST | 6699533 |
| Sequence 9845 | AW293159 | EST | 6699795 |
| Sequence 9846 | AW294649 | EST | 6701285 |
| Sequence 9847 | AW294971 | EST | 6701607 |
| Sequence 9848 | AW296629 | EST | 6703265 |
| Sequence 9849 | AW303456 | EST | 6713145 |
| Sequence 9850 | AW327374 | EST | 6797869 |
| Sequence 9851 | AW327483 | EST | 6797978 |
| Sequence 9852 | AW340172 | EST | 6836798 |
| Sequence 9853 | AW340708 | EST | 6837334 |
| Sequence 9854 | AW360965 | EST | 6865615 |
| Sequence 9855 | AW361001 | EST | 6865651 |
| Sequence 9856 | AW361103 | EST | 6865753 |
| Sequence 9857 | AW361711 | EST | 6866361 |
| Sequence 9858 | AW362982 | EST | 6867632 |
| Sequence 9859 | AW363561 | EST | 6868211 |
| Sequence 9860 | AW364748 | EST | 6869502 |
| Sequence 9861 | AW364827 | EST | 6869386 |
| Sequence 9862 | AW365085 | EST | 6869735 |
| Sequence 9863 | AW365852 | EST | 6870502 |
| Sequence 9864 | AW368293 | EST | 6872943 |
| Sequence 9865 | AW368461 | EST | 6873111 |
| Sequence 9866 | AW375589 | EST | 6880152 |
| Sequence 9867 | AW376917 | EST | 6881580 |
| Sequence 9868 | AW379827 | EST | 6884486 |
| Sequence 9869 | AW381227 | EST | 6885886 |
| Sequence 9870 | AW381248 | EST | 6885907 |
| Sequence 9871 | AW383599 | EST | 6888167 |
| Sequence 9872 | AW384101 | EST | 6888760 |
| Sequence 9873 | AW384915 | EST | 6889574 |
| Sequence 9874 | AW385192 | EST | 6889851 |
| Sequence 9875 | AW385322 | EST | 6889981 |
| Sequence 9876 | AW385328 | EST | 6889987 |
| Sequence 9877 | AW388939 | EST | 6893598 |
| Sequence 9878 | AW392421 | EST | 6897080 |
| Sequence 9879 | AW393095 | EST | 6897754 |
| Sequence 9880 | AW401915 | EST | 6920601 |
| Sequence 9881 | AW402136 | EST | 6920822 |
| Sequence 9882 | AW402676 | EST | 6921388 |
| Sequence 9883 | AW404132 | EST | 6923189 |
| Sequence 9884 | AW405567 | EST | 6924624 |
| Sequence 9885 | AW407212 | EST | 6926269 |
| Sequence 9886 | AW408274 | EST | 6927331 |
| Sequence 9887 | AW408423 | EST | 6927480 |
| Sequence 9888 | AW410520 | EST | 6936061 |
| Sequence 9889 | AW410772 | EST | 6936313 |
| Sequence 9890 | AW411211 | EST | 6936752 |
| Sequence 9891 | AW411217 | EST | 6936758 |
| Sequence 9892 | AW474433 | EST | 7044539 |
| Sequence 9893 | AW498895 | EST | 7137236 |
| Sequence 9894 | AW500117 | EST | 7112425 |
| Sequence 9895 | AW500758 | EST | 7113663 |
| Sequence 9896 | AW501342 | EST | 7114784 |
| Sequence 9897 | AW501373 | EST | 7114844 |
| Sequence 9898 | AW501390 | EST | 7114878 |
| Sequence 9899 | AW501401 | EST | 7114900 |
| Sequence 9900 | AW501421 | EST | 7114936 |
| Sequence 9901 | AW515416 | EST | N/A |
| Sequence 9902 | AW578462 | EST | 7253511 |
| Sequence 9903 | AW578582 | EST | 7253631 |
| Sequence 9904 | AW582604 | EST | 7257653 |
| Sequence 9905 | AW583629 | EST | 7260587 |
| Sequence 9906 | C04057 | EST | 1467308 |
| Sequence 9907 | F06849 | EST | 672465 |
| Sequence 9908 | F09531 | EST | 682064 |
| Sequence 9909 | F11462 | EST | 705760 |
| Sequence 9910 | F12434 | EST | 706790 |
| Sequence 9911 | H01873 | EST | 864806 |
| Sequence 9912 | H08043 | EST | 872865 |
| Sequence 9913 | H08093 | EST | 872915 |
| Sequence 9914 | H09228 | EST | 874050 |
| Sequence 9915 | H09317 | EST | 874139 |
| Sequence 9916 | H09793 | EST | 874615 |
| Sequence 9917 | H10561 | EST | 875383 |
| Sequence 9918 | H10893 | EST | 875713 |
| Sequence 9919 | H11277 | EST | 876097 |
| Sequence 9920 | H15211 | EST | 880031 |
| Sequence 9921 | H16865 | EST | 883105 |
| Sequence 9922 | H22726 | EST | 891421 |
| Sequence 9923 | H26047 | EST | 895170 |
| Sequence 9924 | H29998 | EST | 900908 |
| Sequence 9925 | H30612 | EST | 901522 |
| Sequence 9926 | H62143 | EST | 1014975 |
| Sequence 9927 | H69585 | EST | 1039791 |
| Sequence 9928 | H70297 | EST | 1040503 |
| Sequence 9929 | H86644 | EST | 1068223 |
| Sequence 9930 | H99096 | EST | 1123764 |
| Sequence 9931 | M85706 | EST | 274353 |
| Sequence 9932 | N26971 | EST | 1141319 |
| Sequence 9933 | N28905 | EST | 1147141 |
| Sequence 9934 | N28944 | EST | 1147180 |
| Sequence 9935 | N34003 | EST | 1154403 |
| Sequence 9936 | N34221 | EST | 1155363 |
| Sequence 9937 | N36764 | EST | 1157906 |
| Sequence 9938 | N42722 | EST | 1167152 |
| Sequence 9939 | N43012 | EST | 1166756 |
| Sequence 9940 | N46795 | EST | 1187961 |
| Sequence 9941 | N54326 | EST | 1195646 |
| Sequence 9942 | N57253 | EST | 1201143 |
| Sequence 9943 | N95531 | EST | 1267800 |
| Sequence 9944 | R17855 | EST | 771465 |
| Sequence 9945 | R19920 | EST | 774554 |
| Sequence 9946 | R34894 | EST | 791795 |
| Sequence 9947 | R35355 | EST | 792256 |
| Sequence 9948 | R35487 | EST | 792388 |
| Sequence 9949 | R50695 | EST | 812597 |
| Sequence 9950 | R52763 | EST | 814665 |
| Sequence 9951 | R53255 | EST | 815157 |
| Sequence 9952 | R59084 | EST | 829779 |
| Sequence 9953 | R69898 | EST | 843415 |
| Sequence 9954 | R73759 | EST | 848129 |
| Sequence 9955 | R84545 | EST | 942951 |
| Sequence 9956 | R88895 | EST | 953722 |
| Sequence 9957 | R89026 | EST | 953853 |
| Sequence 9958 | R93920 | EST | 969315 |
| Sequence 9959 | T05548 | EST | 316698 |
| Sequence 9960 | T08921 | EST | 389949 |
| Sequence 9961 | T11899 | EST | 596603 |
| Sequence 9962 | T18975 | EST | 601018 |
| Sequence 9963 | T19025 | EST | 601068 |
| Sequence 9964 | T32930 | EST | 615028 |
| Sequence 9965 | T63081 | EST | 666738 |
| Sequence 9966 | T77019 | EST | 694222 |
| Sequence 9967 | T84756 | EST | 713108 |
| Sequence 9968 | W02349 | EST | 1274356 |
| Sequence 9969 | W03691 | EST | 1275555 |
| Sequence 9970 | W07619 | EST | 1281621 |
| Sequence 9971 | W27890 | EST | 1307838 |
| Sequence 9972 | W28867 | EST | 1308878 |
| Sequence 9973 | W38913 | EST | 1320619 |
| Sequence 9974 | W38916 | EST | 1320622 |
| Sequence 9975 | W39482 | EST | 1321190 |
| Sequence 9976 | W52153 | EST | 1349763 |
| Sequence 9977 | W60983 | EST | 1367742 |
| Sequence 9978 | W69356 | EST | 1378667 |
| Sequence 9979 | W72044 | EST | 1382314 |
| Sequence 9980 | W76216 | EST | 1386441 |
| Sequence 9981 | W79237 | EST | 1390368 |
| Sequence 9982 | W80613 | EST | 1391700 |
| Sequence 9983 | W81613 | EST | 1392652 |
| Sequence 9984 | W88815 | EST | 1404287 |
| Sequence 9985 | W95032 | EST | 1424152 |
| Sequence 9986 | Z25224 | EST | 395863 |
| Sequence 9987 | Z42427 | EST | 565844 |
| Sequence 9988 | Z45029 | EST | 574221 |
| Sequence 9989 | Z45988 | EST | 575222 |
| Sequence 9990 | Z78343 | EST | 1495116 |
| Sequence 9991 | Q46540 | NUCPATENT | N/A |
| Sequence 9992 | Q84560 | NUCPATENT | N/A |
| Sequence 9993 | T33624 | NUCPATENT | N/A |
| Sequence 9994 | T71086 | NUCPATENT | N/A |
| Sequence 9995 | T94119 | NUCPATENT | N/A |
| Sequence 9996 | V04699 | NUCPATENT | N/A |
| Sequence 9997 | V05728 | NUCPATENT | N/A |
| Sequence 9998 | V34175 | NUCPATENT | N/A |
| Sequence 9999 | V34185 | NUCPATENT | N/A |
| Sequence 10000 | V35457 | NUCPATENT | N/A |
| Sequence 10001 | V36303 | NUCPATENT | N/A |
| Sequence 10002 | V41451 | NUCPATENT | N/A |
| Sequence 10003 | V58631 | NUCPATENT | N/A |
| Sequence 10004 | V84411 | NUCPATENT | N/A |
| Sequence 10005 | V84546 | NUCPATENT | N/A |
| Sequence 10006 | V87058 | NUCPATENT | N/A |
| Sequence 10007 | V89685 | NUCPATENT | N/A |
| Sequence 10008 | V90259 | NUCPATENT | N/A |
| Sequence 10009 | V99828 | NUCPATENT | N/A |
| Sequence 10010 | X00616 | NUCPATENT | N/A |
| Sequence 10011 | X05715 | NUCPATENT | N/A |
| Sequence 10012 | X37479 | NUCPATENT | N/A |
| Sequence 10013 | X40381 | NUCPATENT | N/A |
| Sequence 10014 | X61430 | NUCPATENT | N/A |
| Sequence 10015 | X77342 | NUCPATENT | N/A |
| Sequence 10016 | X80672 | NUCPATENT | N/A |
| Sequence 10017 | X87868 | NUCPATENT | N/A |
| Sequence 10018 | X91484 | NUCPATENT | N/A |
| Sequence 10019 | X97576 | NUCPATENT | N/A |
| Sequence 10020 | X97718 | NUCPATENT | N/A |
| Sequence 10021 | X97772 | NUCPATENT | N/A |
| Sequence 10022 | X98309 | NUCPATENT | N/A |
| Sequence 10023 | X98400 | NUCPATENT | N/A |
| Sequence 10024 | X98781 | NUCPATENT | N/A |
| Sequence 10025 | X99117 | NUCPATENT | N/A |
| Sequence 10026 | Z00428 | NUCPATENT | N/A |
| Sequence 10027 | Z14314 | NUCPATENT | N/A |
| Sequence 10028 | Z14685 | NUCPATENT | N/A |
| Sequence 10029 | Z15023 | NUCPATENT | N/A |
| Sequence 10030 | Z24893 | NUCPATENT | N/A |
| Sequence 10031 | Z27237 | NUCPATENT | N/A |
| Sequence 10032 | Z29638 | NUCPATENT | N/A |
| Sequence 10033 | Z38853 | NUCPATENT | N/A |
| Sequence 10034 | Z39903 | NUCPATENT | N/A |
| Sequence 10035 | Z41312 | NUCPATENT | N/A |
| Sequence 10036 | Z52414 | NUCPATENT | N/A |
| Sequence 10037 | Z52416 | NUCPATENT | N/A |
| Sequence 10038 | Z53013 | NUCPATENT | N/A |
| Sequence 10039 | Z57842 | NUCPATENT | N/A |
| Sequence 10040 | Z60383 | NUCPATENT | N/A |
| Sequence 10041 | Z65271 | NUCPATENT | N/A |
| Sequence 10042 | Z80193 | NUCPATENT | N/A |
| Sequence 10043 | Z80472 | NUCPATENT | N/A |
| Sequence 10044 | Z97065 | NUCPATENT | N/A |
| Sequence 10045 | Z97145 | NUCPATENT | N/A |
| Sequence 10046 | Z97322 | NUCPATENT | N/A |
| Sequence 10047 | AC05588 | PREPATNUC | N/A |
| Sequence 10048 | AC37772 | PREPATNUC | N/A |
| Sequence 10049 | AC38092 | PREPATNUC | N/A |
| Sequence 10050 | AC38120 | PREPATNUC | N/A |
| Sequence 10051 | AC38948 | PREPATNUC | N/A |
| Sequence 10052 | AC39251 | PREPATNUC | N/A |
| Sequence 10053 | AC39528 | PREPATNUC | N/A |
| Sequence 10054 | AC39561 | PREPATNUC | N/A |

TABLE 3-4
Sequence 10047: Found in patent publication WO99/09061
CCCGCGGTGGCGGCCGAGGTACTGCGTGGCCGTCCGCTGTTGTGTGGCCTGGGCCGCAA
A
CCGGGCCACGTGGTTGACAATGGGGGAGAAATTGGTAGGACCGTAGAAGCGGATGTGGGG
CAGGCAAGCTGAGTACGCCTGGGCAATACCATCCACACCTGAGCAGAAGGGGTTGGTGGG
GTTGAAGTTGATGGCAAACTCATGGGAGACCTTCCAGTCTGGGGGTAACTGGGCCCCGAA
TCCCAGAGCTGGAAACATCTTATCACTGTCGTAGTCCTGAATGATCTGCCCAACAGCCCA
GATGGCCGACAGATATTCGTTGGTGCCCATAGGGTTGATATANTGCAAAGAGGAAGGGTC
GAGGGGATTCCCGTTGGAGGCTGTAAAGTCTATTCCAACGGTGAACATGAGCTGGCAGCC
TCCCAGGACGTAGTCAAGGAAGGAGTAGT
Sequence 10048: Found in patent publication WO00/06698
CCGCGGTGGCGGCCGAGGTCTGAACTGCGGGGTCTCTATCGCACTGCTAGGGGTTCTGCT
GCTGGGTGCGGCGCGCCTGCCGCGCGGGGCAGAAGCTTTTGAGATTGCTCTGCCACGAGA
AAGCAACATTACAGTTCTCATAAAGCTGGGGACCCCGACTCTGCTGGCAAAACCCTGTTA
CATCGTCTTTTCTAAAAGACATATAACCATGTTGTCCATCAAGTCTGGAGAAAGAATAGT
CTTTACCTTTAGCTGCCAGAGTCCTGAGAATCACTTTGTCATANAGATCCAGAAAAATAT
TGACTGTATGTCAGGCCCATGTCCTTTTGGGGAGGTTCAGCTTCAGCCCTCGACATCGTT
GTTGCCTACCCTCAACAGAACTTTCATCTGGGATGTCAAA
Sequence 10049: Found in patent publication WO00/06698
TGATCTTCAGCTGCTCCTGAACTTTGGCACTGTAAGGGTTCTCTGTATCGTATGGTGGTG
ACAAAGCTTTTAAAATAACCTNTCCTCCAGTGCATGGAAAAGGACTGGAGTATTTATNAA
GT
Sequence 10050: Found in patent publication WO00/06730
ACTTGTGAGCCTGGACTGGCTTCCCCGGCCTCCTACATGAACCCCTTCCCGGTGCTCCAT
CTCATCGAGGACTTGAGGCTGGCCTTGGAGATGCTGGAGCTTCCTCAGGAGAGAGCAGCC
CTCCTGAGCCAGATCCCTGGCCCAACAGCTGCCTACATAAAGGAATGGTTTGAAGAGAGC
TTGTCCCAGGTAAGCCACCACAGTGCTGCTAGTAATGAAACCTACCAGGAACGCGTTGGCA
CGTCTAGAAGGGGATAAGGAGTCCCTCATATTGCAGGTGAGTGTCCTCACAGACCAAGTA
GAAGCCCAGGGAGAAAAGATTCGAGACCTGGAAGTGTGTCTGGAAGGACACCAGGTGAAA
CTCAATGCTGCTGAAGAGATGCTTCAACAGGAGCTGCTAAGCCGCACATCTCTTGAGACC
CAGAAGCTCGATCTGATGACTGAAGTGTCTGAGCTGAAGCTCAAGCTGGTTGGCATGGAG
AAGGAGCAGAGAGAGCAGGAGGAGAAGCAGAGAAAAGCAGAGGAGTTACTGCAAGAGCTC
AGGCCCCTCAAAATCAAAGTGGAAGAGTTTGGAAA
Sequence 10051: Found in patent publication WO00/11171
GGGAGTGGAGCGGTGAACACGTCAGGGGTGGGGGGCGCAGGTCAAGCTTTCACCAGTTTT
TAATTCTTTGATGGGGTAAATTTGAGCAATTTTCTCGACTTGTCGACATTCGTTATTAAC
TGANCAGGAATCAGGANAGGAACCCGGTCCTCTCCACACAGCCCAGCANAGAGCCTACGA
CTAGATTTGCATCTTTACGTCCTGCGCGGAGGCTGCTACACACATGCANAAGTCATGCTG
GTGGCCTGGACATTGAAGGGAGAGAAGTGGATTTGGGAGACATTTAGGAGGCACCGAAAG
CGAAGGAAGCTCCTGCTCCTCCTAAAGCCGAAGCCAAAGCAAAGGCTTTAAAGGCCAAGA
AGGCAGTGTTGAAAGGTGTCCGCAGCCACACGCAAAAAAGAGGATCCGCATGTCACTCAC
CT
Sequence 10052: Found in patent publication WO00/15799
ACCGCGGGGGCGGCCGNGGNTTCNGCCTGGAGGAGCTCAGGGTGGCCGGCATTCACAAG
A
AGGTGGCCCGGACCATCGGCATTTCTGTGGATCCGAGGAGGCGGAACAAGTCCACGGAGT
CCCTGCAGACCAACGTGCAGCGGCTGAAGGAGTACCACAAAGGGACCCAAATTCAGCGGT
CTGTGCCTACAAACTTCATTAATAACTGCTTGCAGATTGGCAGCTATCTGGTCACTTGAC
ATATCCAATGTTGCTATTTTGGTCTGGAGAAAGTTCTCCCTTTCTTCATCTACCTTAATT
TCATGTCCATTTTTAAATAATTCAAACATTTTGGGGATGTCACGGCCAATGGAATTTCGA
GAAAGCTTTGGATATTTTTTATTCAGTTTCTTCCTTAATCGATTAAGTTCAGGCATTATT
TCTGGAACAGCTACGTAAAAGTCTGCAACAATTTCATCATCCCAAATCTTCTGTATCAGA
CTAAGTGCCTCCTGCAAATGCAGCTCCATTTTCTTCCGCTATTTTGACCTCTGATGCATT
CTCTGTAAATACAGCAACTTTATTGATTTCCGAAGCCAATGGGTATGGCAAACTAANAAC
ACTGGTAAATGGCTTCACGTTTTTCTTNTTTTCCAGTGCCATATNCAGTGTCAAAATCAA
ANAAAACACTTTGCTTTGGACTAGTTAAAGCCAANAATTTGA
Sequence 10053: Found in patent publication WO00/06698
ACATCTTCCAGGACAAGGTAAACTCTGACATGCACTAGGTATGTGCAGATCCCGGCCCCT
GCCACCCAGCCTCATGCAAGTCATCCCCGACATGACCTTCACGACCGCAATGCAAGGAGG
GGAAGAAAGTCACAGCACTGATGAGGACAGCTGCAGAGGTGGCAGTGTGTGGACACAGGA
AGTTTGGGCCCCCTCCCTGCCCCAGCTTTCCTAGGCCAGAATTGTGTTTGGCAGTAATTG
TCTGTTTAAAAAAATAAAAAGGAGAGGAAGCGTTCACCGCCACAAATCATAAAATGGACA
TGACTGTGGAGTCTTACAGTTCAGGGTTCTTTCATTCACGTCCCTTCCTGTCTCGGTCTG
CGGTCTTTACCACATCAATAGGACTTTTTATGCGTCCGGGTTAATTTTTCACTCCAGTGC
GTCCTGTTGCAGGGACCGGAGCTGATGGGAGCTGCTTCTCCCCATGCCTCACTGGTCCCA
GATCANGGCTTCAGGGACAGATGATGAGTCTCAAACGAGCCANCAGGGGTTCTTTTGGGT
ATAAATGGGCAATTCGNCCTGTCTTAAGNCTGATGACCTCANCCGTGGTTTTTGGAT
Sequence 10054: Found in patent publication WO00/18922
TAGGGCGAATTGGAGCTCCCCGCGGTGGCGGCCGAGGTACCTGAGCCAGGAGGAGGCCC
A
GGCCGTGGACCAGGAGCTATTTAACGAATACCAGTTCAGCGTGGACCAACTTATGGAACT
GGCCGGGCTGAGCTGTGCTACAGCCATCGCCAAGGCATATCCCCCCACGTCCATGTCCAG
GAGCCCCCCTACTGTCCTGGTCATCTGTGGCCCGGGGAATAATGGAGGAGATGGTCTGGT
CTGTGCTCGACACCTCAAACTCTTTGGCTACGAGCCAACCATCTATTACCCCAAAAGGCC
TAACAAGCCCCTCTTCACTGCATTGGTGACCCAGTGTCAGAAAATGGACATCCCTTTCCT
TGGGGAAATGCCCGCAGAGCCCATGACGATTGATGAACTGTTGAGCTGGTGGTGGATGCC
ATCTTTGGCTTCAGCTTCAAGGGCGATGTTCGGGAAC

TABLE 3-5

| | | | |
|---|---|---|---|
| Sequence 10110 | AB025959 | Genbank | 6520072 |
| Sequence 10111 | AB026894 | Genbank | 5821423 |
| Sequence 10112 | AB027466 | Genbank | 6172220 |
| Sequence 10113 | AB028624 | Genbank | 5103045 |
| Sequence 10114 | AB028859 | Genbank | 6567165 |
| Sequence 10115 | AB028893 | Genbank | 6552364 |
| Sequence 10116 | AB028975 | Genbank | 5689440 |
| Sequence 10117 | AB029001 | Genbank | 5689492 |
| Sequence 10118 | AB029025 | Genbank | 5689540 |
| Sequence 10119 | AB031052 | Genbank | 6978947 |
| Sequence 10120 | AB032957 | Genbank | 6329818 |
| Sequence 10121 | AB032959 | Genbank | 6329832 |
| Sequence 10122 | AB032982 | Genbank | 6330122 |
| Sequence 10123 | AB033017 | Genbank | 6330344 |
| Sequence 10124 | AB033050 | Genbank | 6330623 |
| Sequence 10125 | AB033074 | Genbank | 6330846 |
| Sequence 10126 | AB033079 | Genbank | 6382025 |
| Sequence 10127 | AB034205 | Genbank | 6899845 |
| Sequence 10128 | AB034206 | Genbank | 6177737 |
| Sequence 10129 | AB035207 | Genbank | 6469033 |
| Sequence 10130 | AB037740 | Genbank | 7243018 |
| Sequence 10131 | AB037763 | Genbank | 7243064 |
| Sequence 10132 | AB037767 | Genbank | 7243072 |
| Sequence 10133 | AB037817 | Genbank | 7243172 |
| Sequence 10134 | AB037850 | Genbank | 7243238 |
| Sequence 10135 | AC000049 | Genbank | 5705995 |
| Sequence 10136 | AC000064 | Genbank | 1669369 |
| Sequence 10137 | AC000080 | Genbank | 7123907 |
| Sequence 10138 | AC000120 | Genbank | 1809224 |
| Sequence 10139 | AC000353 | Genbank | 6970735 |
| Sequence 10140 | AC000378 | Genbank | 2270906 |
| Sequence 10141 | AC000394 | Genbank | 2133911 |
| Sequence 10142 | AC001226 | Genbank | 2133862 |
| Sequence 10143 | AC001642 | Genbank | 1945597 |
| Sequence 10144 | AC002038 | Genbank | 2226439 |
| Sequence 10145 | AC002039 | Genbank | 2342716 |
| Sequence 10146 | AC002051 | Genbank | 7798752 |
| Sequence 10147 | AC002064 | Genbank | 2076723 |
| Sequence 10148 | AC002347 | Genbank | 2828783 |
| Sequence 10149 | AC002350 | Genbank | 2961443 |
| Sequence 10150 | AC002357 | Genbank | 2815516 |
| Sequence 10151 | AC002400 | Genbank | 2576344 |
| Sequence 10152 | AC002470 | Genbank | 6478944 |
| Sequence 10153 | AC002528 | Genbank | 2388554 |
| Sequence 10154 | AC002531 | Genbank | 2580476 |
| Sequence 10155 | AC002544 | Genbank | 3337382 |
| Sequence 10156 | AC002553 | Genbank | 3126783 |
| Sequence 10157 | AC002984 | Genbank | 2443872 |
| Sequence 10158 | AC003004 | Genbank | 2522284 |
| Sequence 10159 | AC003007 | Genbank | 2911728 |
| Sequence 10160 | AC003012 | Genbank | 2547258 |
| Sequence 10161 | AC003025 | Genbank | 3337308 |
| Sequence 10162 | AC003047 | Genbank | 3169149 |
| Sequence 10163 | AC003103 | Genbank | 2842782 |
| Sequence 10164 | AC003111 | Genbank | 2638670 |
| Sequence 10165 | AC003663 | Genbank | 3097871 |
| Sequence 10166 | AC003688 | Genbank | 3789719 |
| Sequence 10167 | AC003692 | Genbank | 2731601 |
| Sequence 10168 | AC003973 | Genbank | 2739352 |
| Sequence 10169 | AC004008 | Genbank | 2781388 |
| Sequence 10170 | AC004036 | Genbank | 2811104 |
| Sequence 10171 | AC004041 | Genbank | 2811099 |
| Sequence 10172 | AC004067 | Genbank | 3851208 |
| Sequence 10173 | AC004084 | Genbank | 2822156 |
| Sequence 10174 | AC004111 | Genbank | 2828786 |
| Sequence 10175 | AC004148 | Genbank | 3482960 |
| Sequence 10176 | AC004151 | Genbank | 2896795 |
| Sequence 10177 | AC004213 | Genbank | 3386628 |
| Sequence 10178 | AC004228 | Genbank | 4263838 |
| Sequence 10179 | AC004236 | Genbank | 2914668 |
| Sequence 10180 | AC004258 | Genbank | 2924759 |
| Sequence 10181 | AC004386 | Genbank | 3046272 |
| Sequence 10182 | AC004388 | Genbank | 3046271 |
| Sequence 10183 | AC004453 | Genbank | 2979600 |
| Sequence 10184 | AC004496 | Genbank | 2996645 |
| Sequence 10185 | AC004527 | Genbank | 4760422 |
| Sequence 10186 | AC004530 | Genbank | 3108050 |
| Sequence 10187 | AC004551 | Genbank | 3309005 |
| Sequence 10188 | AC004582 | Genbank | 3337307 |
| Sequence 10189 | AC004584 | Genbank | 3417305 |
| Sequence 10190 | AC004596 | Genbank | 4210521 |
| Sequence 10191 | AC004637 | Genbank | 3094998 |
| Sequence 10192 | AC004668 | Genbank | 3115345 |
| Sequence 10193 | AC004682 | Genbank | 3337388 |
| Sequence 10194 | AC004687 | Genbank | 3258613 |
| Sequence 10195 | AC004707 | Genbank | 3249127 |
| Sequence 10196 | AC004742 | Genbank | 3152630 |
| Sequence 10197 | AC004757 | Genbank | 3341707 |
| Sequence 10198 | AC004810 | Genbank | 3264567 |
| Sequence 10199 | AC004816 | Genbank | 4156209 |
| Sequence 10200 | AC004821 | Genbank | 4753291 |
| Sequence 10201 | AC004832 | Genbank | 6624129 |
| Sequence 10202 | AC004835 | Genbank | 4508154 |
| Sequence 10203 | AC004854 | Genbank | 4827328 |
| Sequence 10204 | AC004865 | Genbank | 4156195 |
| Sequence 10205 | AC004878 | Genbank | 3213119 |
| Sequence 10206 | AC004884 | Genbank | 4156189 |
| Sequence 10207 | AC004904 | Genbank | 4156180 |
| Sequence 10208 | AC004908 | Genbank | 4156179 |
| Sequence 10209 | AC004913 | Genbank | 3213084 |
| Sequence 10210 | AC004918 | Genbank | 4153871 |
| Sequence 10211 | AC004935 | Genbank | 4156170 |
| Sequence 10212 | AC004938 | Genbank | 3213059 |
| Sequence 10213 | AC004943 | Genbank | 3924671 |
| Sequence 10214 | AC004955 | Genbank | 4753267 |
| Sequence 10215 | AC004968 | Genbank | 4156160 |
| Sequence 10216 | AC004969 | Genbank | 4156159 |
| Sequence 10217 | AC004982 | Genbank | 3419846 |
| Sequence 10218 | AC004983 | Genbank | 4309885 |
| Sequence 10219 | AC004985 | Genbank | 5708490 |
| Sequence 10220 | AC004999 | Genbank | 3970963 |
| Sequence 10221 | AC005023 | Genbank | 3900847 |
| Sequence 10222 | AC005035 | Genbank | 3953506 |
| Sequence 10223 | AC005041 | Genbank | 4508118 |
| Sequence 10224 | AC005057 | Genbank | 6587915 |
| Sequence 10225 | AC005058 | Genbank | 4156135 |
| Sequence 10226 | AC005069 | Genbank | 4753224 |
| Sequence 10227 | AC005070 | Genbank | 3406049 |
| Sequence 10228 | AC005071 | Genbank | 4508112 |
| Sequence 10229 | AC005095 | Genbank | 4753220 |
| Sequence 10230 | AC005101 | Genbank | 4508108 |
| Sequence 10231 | AC005154 | Genbank | 3242763 |
| Sequence 10232 | AC005180 | Genbank | 3687281 |
| Sequence 10233 | AC005189 | Genbank | 3264580 |
| Sequence 10234 | AC005193 | Genbank | 3309101 |
| Sequence 10235 | AC005203 | Genbank | 3273381 |
| Sequence 10236 | AC005212 | Genbank | 3287444 |
| Sequence 10237 | AC005218 | Genbank | 3282162 |
| Sequence 10238 | AC005258 | Genbank | 3289992 |
| Sequence 10239 | AC005263 | Genbank | 3289978 |
| Sequence 10240 | AC005300 | Genbank | 6492490 |
| Sequence 10241 | AC005316 | Genbank | 3738342 |
| Sequence 10242 | AC005318 | Genbank | 3885345 |
| Sequence 10243 | AC005325 | Genbank | 3366581 |
| Sequence 10244 | AC005343 | Genbank | 4165008 |
| Sequence 10245 | AC005346 | Genbank | 3366566 |
| Sequence 10246 | AC005368 | Genbank | 3367506 |
| Sequence 10247 | AC005378 | Genbank | 4508139 |
| Sequence 10248 | AC005412 | Genbank | 5931398 |
| Sequence 10249 | AC005480 | Genbank | 4835818 |
| Sequence 10250 | AC005488 | Genbank | 5836194 |
| Sequence 10251 | AC005520 | Genbank | 5001541 |
| Sequence 10252 | AC005531 | Genbank | 4153875 |
| Sequence 10253 | AC005550 | Genbank | 3478668 |
| Sequence 10254 | AC005606 | Genbank | 3540172 |
| Sequence 10255 | AC005612 | Genbank | 3540153 |
| Sequence 10256 | AC005618 | Genbank | 3548785 |
| Sequence 10257 | AC005630 | Genbank | 4159882 |
| Sequence 10258 | AC005661 | Genbank | 3818348 |
| Sequence 10259 | AC005740 | Genbank | 3687210 |
| Sequence 10260 | AC005754 | Genbank | 3688074 |
| Sequence 10261 | AC005786 | Genbank | 3702287 |
| Sequence 10262 | AC005789 | Genbank | 3702281 |
| Sequence 10263 | AC005799 | Genbank | 3789715 |
| Sequence 10264 | AC005828 | Genbank | 3789713 |
| Sequence 10265 | AC005876 | Genbank | 6249672 |
| Sequence 10266 | AC005971 | Genbank | 4314421 |
| Sequence 10267 | AC006001 | Genbank | 5708496 |
| Sequence 10268 | AC006008 | Genbank | 5091655 |
| Sequence 10269 | AC006017 | Genbank | 4508141 |
| Sequence 10270 | AC006040 | Genbank | 4314426 |
| Sequence 10271 | AC006055 | Genbank | 4071011 |
| Sequence 10272 | AC006057 | Genbank | 4731048 |
| Sequence 10273 | AC006059 | Genbank | 4544348 |
| Sequence 10274 | AC006064 | Genbank | 4572650 |
| Sequence 10275 | AC006116 | Genbank | 3962497 |
| Sequence 10276 | AC006160 | Genbank | 5701616 |
| Sequence 10277 | AC006254 | Genbank | 4726099 |
| Sequence 10278 | AC006255 | Genbank | 5306223 |
| Sequence 10279 | AC006257 | Genbank | 4092478 |
| Sequence 10280 | AC006261 | Genbank | 4079613 |
| Sequence 10281 | AC006312 | Genbank | 4582483 |
| Sequence 10282 | AC006347 | Genbank | 4309919 |
| Sequence 10283 | AC006389 | Genbank | 4309883 |
| Sequence 10284 | AC006445 | Genbank | 6721147 |
| Sequence 10285 | AC006454 | Genbank | 5732182 |
| Sequence 10286 | AC006461 | Genbank | 4508128 |
| Sequence 10287 | AC006477 | Genbank | 7243871 |
| Sequence 10288 | AC006485 | Genbank | 4206211 |
| Sequence 10289 | AC006487 | Genbank | 4314418 |
| Sequence 10290 | AC006501 | Genbank | 4309874 |
| Sequence 10291 | AC006504 | Genbank | 4220442 |
| Sequence 10292 | AC006509 | Genbank | 5815507 |
| Sequence 10293 | AC006518 | Genbank | 4713939 |
| Sequence 10294 | AC006543 | Genbank | 4388738 |
| Sequence 10295 | AC006561 | Genbank | 4558534 |
| Sequence 10296 | AC006581 | Genbank | 4914350 |
| Sequence 10297 | AC006960 | Genbank | 4337211 |
| Sequence 10298 | AC007041 | Genbank | 5708471 |
| Sequence 10299 | AC007055 | Genbank | 4885691 |
| Sequence 10300 | AC007191 | Genbank | 4558643 |
| Sequence 10301 | AC007204 | Genbank | 4559317 |
| Sequence 10302 | AC007225 | Genbank | 6715703 |
| Sequence 10303 | AC007226 | Genbank | 6715702 |
| Sequence 10304 | AC007227 | Genbank | 8456148 |
| Sequence 10305 | AC007325 | Genbank | 7229741 |
| Sequence 10306 | AC007528 | Genbank | 5263309 |
| Sequence 10307 | AC007546 | Genbank | 5668756 |
| Sequence 10308 | AC007637 | Genbank | 8439781 |
| Sequence 10309 | AC007684 | Genbank | 5836173 |
| Sequence 10310 | AC007708 | Genbank | 5923689 |
| Sequence 10311 | AC007790 | Genbank | 5042474 |
| Sequence 10312 | AC007842 | Genbank | 5080755 |
| Sequence 10313 | AC007967 | Genbank | 5931455 |
| Sequence 10314 | AC008082 | Genbank | 6143846 |
| Sequence 10315 | AC008101 | Genbank | 6970743 |
| Sequence 10316 | AC008116 | Genbank | 6001959 |
| Sequence 10317 | AC008124 | Genbank | 5815491 |
| Sequence 10318 | AC008179 | Genbank | 5931440 |
| Sequence 10319 | AC008269 | Genbank | 7243890 |
| Sequence 10320 | AC008498 | Genbank | 6850299 |
| Sequence 10321 | AC008893 | Genbank | 7259691 |
| Sequence 10322 | AC009363 | Genbank | 6648145 |
| Sequence 10323 | AC009509 | Genbank | 6492473 |
| Sequence 10324 | AC009946 | Genbank | 6604542 |
| Sequence 10325 | AC011331 | Genbank | 9929686 |
| Sequence 10326 | AC011362 | Genbank | 6094545 |
| Sequence 10327 | AC015853 | Genbank | 6721261 |
| Sequence 10328 | AC020663 | Genbank | 6682593 |
| Sequence 10329 | AD000092 | Genbank | 1905905 |
| Sequence 10330 | AE000658 | Genbank | 2358019 |
| Sequence 10331 | AF000160 | Genbank | 2306783 |
| Sequence 10332 | AF001437 | Genbank | 2316039 |
| Sequence 10333 | AF001549 | Genbank | 3355302 |
| Sequence 10334 | AF003001 | Genbank | 2529443 |
| Sequence 10335 | AF005888 | Genbank | 2738487 |
| Sequence 10336 | AF006043 | Genbank | 2674061 |
| Sequence 10337 | AF006088 | Genbank | 2282041 |
| Sequence 10338 | AF006501 | Genbank | 4836840 |
| Sequence 10339 | AF007140 | Genbank | 2852616 |
| Sequence 10340 | AF007544 | Genbank | 2970122 |
| Sequence 10341 | AF010238 | Genbank | 2282063 |
| Sequence 10342 | AF011390 | Genbank | 3298567 |
| Sequence 10343 | AF012072 | Genbank | 9967556 |
| Sequence 10344 | AF014807 | Genbank | 2338731 |
| Sequence 10345 | AF014955 | Genbank | 2772828 |
| Sequence 10346 | AF015812 | Genbank | 2599359 |
| Sequence 10347 | AF020351 | Genbank | 2655052 |
| Sequence 10348 | AF020352 | Genbank | 2655054 |
| Sequence 10349 | AF021819 | Genbank | 2460317 |
| Sequence 10350 | AF022211 | Genbank | 2465723 |
| Sequence 10351 | AF026126 | Genbank | 2828601 |
| Sequence 10352 | AF026292 | Genbank | 2559009 |
| Sequence 10353 | AF028832 | Genbank | 3287488 |
| Sequence 10354 | AF029786 | Genbank | 3403166 |
| Sequence 10355 | AF031416 | Genbank | 3213216 |
| Sequence 10356 | AF032387 | Genbank | 2641556 |
| Sequence 10357 | AF033095 | Genbank | 2645728 |
| Sequence 10358 | AF034759 | Genbank | 2653648 |
| Sequence 10359 | AF035286 | Genbank | 2661038 |
| Sequence 10360 | AF035295 | Genbank | 2661050 |
| Sequence 10361 | AF035313 | Genbank | 2661075 |
| Sequence 10362 | AF037222 | Genbank | 2921499 |
| Sequence 10363 | AF037447 | Genbank | 6466790 |
| Sequence 10364 | AF037448 | Genbank | 3037012 |
| Sequence 10365 | AF038196 | Genbank | 2795917 |
| Sequence 10366 | AF038200 | Genbank | 2795921 |
| Sequence 10367 | AF038554 | Genbank | 2895558 |
| Sequence 10368 | AF038954 | Genbank | 3329377 |
| Sequence 10369 | AF038955 | Genbank | 3329379 |
| Sequence 10370 | AF038958 | Genbank | 3329385 |
| Sequence 10371 | AF038966 | Genbank | 2791681 |
| Sequence 10372 | AF039029 | Genbank | 3834389 |
| Sequence 10373 | AF042836 | Genbank | 3776241 |
| Sequence 10374 | AF042857 | Genbank | 2952271 |
| Sequence 10375 | AF044956 | Genbank | 5326827 |
| Sequence 10376 | AF044957 | Genbank | 4164445 |
| Sequence 10377 | AF044959 | Genbank | 3348136 |
| Sequence 10378 | AF047181 | Genbank | 2909853 |
| Sequence 10379 | AF047431 | Genbank | 5514630 |
| Sequence 10380 | AF047436 | Genbank | 3335127 |
| Sequence 10381 | AF047439 | Genbank | 3335133 |
| Sequence 10382 | AF047440 | Genbank | 3335135 |
| Sequence 10383 | AF048686 | Genbank | 5737698 |
| Sequence 10384 | AF049140 | Genbank | 2947300 |
| Sequence 10385 | AF049613 | Genbank | 3329428 |
| Sequence 10386 | AF050637 | Genbank | 4164451 |
| Sequence 10387 | AF050638 | Genbank | 5326819 |
| Sequence 10388 | AF050639 | Genbank | 4164453 |
| Sequence 10389 | AF051894 | Genbank | 3095110 |
| Sequence 10390 | AF052101 | Genbank | 3360408 |
| Sequence 10391 | AF052129 | Genbank | 3360438 |
| Sequence 10392 | AF052642 | Genbank | 2981453 |
| Sequence 10393 | AF053356 | Genbank | 3135305 |
| Sequence 10394 | AF053551 | Genbank | 3283048 |
| Sequence 10395 | AF053719 | Genbank | 6670884 |
| Sequence 10396 | AF054187 | Genbank | 4092059 |
| Sequence 10397 | AF054990 | Genbank | 3005703 |
| Sequence 10398 | AF054996 | Genbank | 3005711 |
| Sequence 10399 | AF055029 | Genbank | 3005759 |
| Sequence 10400 | AF055460 | Genbank | 3335143 |
| Sequence 10401 | AF055473 | Genbank | nil |
| Sequence 10402 | AF063020 | Genbank | 3283351 |
| Sequence 10403 | AF064019 | Genbank | 3347856 |
| Sequence 10404 | AF064607 | Genbank | 3152667 |
| Sequence 10405 | AF067518 | Genbank | 3850315 |
| Sequence 10406 | AF067656 | Genbank | 3901271 |
| Sequence 10407 | AF067844 | Genbank | 4240386 |
| Sequence 10408 | AF068846 | Genbank | 3201999 |
| Sequence 10409 | AF069378 | Genbank | 3982733 |
| Sequence 10410 | AF070598 | Genbank | 3387976 |
| Sequence 10411 | AF070635 | Genbank | 3283905 |
| Sequence 10412 | AF070644 | Genbank | 3283917 |
| Sequence 10413 | AF070649 | Genbank | 3283923 |
| Sequence 10414 | AF070655 | Genbank | 4454685 |
| Sequence 10415 | AF070659 | Genbank | 4454693 |
| Sequence 10416 | AF073298 | Genbank | 3641537 |
| Sequence 10417 | AF077030 | Genbank | 4689107 |
| Sequence 10418 | AF077036 | Genbank | 4689119 |
| Sequence 10419 | AF077037 | Genbank | 4689121 |
| Sequence 10420 | AF077043 | Genbank | 4689133 |
| Sequence 10421 | AF077202 | Genbank | 4679017 |
| Sequence 10422 | AF080092 | Genbank | 4322311 |
| Sequence 10423 | AF081484 | Genbank | 3420928 |
| Sequence 10424 | AF082513 | Genbank | 3649656 |
| Sequence 10425 | AF083441 | Genbank | 5813822 |
| Sequence 10426 | AF085357 | Genbank | 5114048 |
| Sequence 10427 | AF085359 | Genbank | 5114052 |
| Sequence 10428 | AF085867 | Genbank | 3483188 |
| Sequence 10429 | AF086090 | Genbank | 3483435 |
| Sequence 10430 | AF086207 | Genbank | 3483552 |
| Sequence 10431 | AF086210 | Genbank | 3483555 |
| Sequence 10432 | AF086234 | Genbank | 3483579 |
| Sequence 10433 | AF086378 | Genbank | 3483723 |
| Sequence 10434 | AF086388 | Genbank | 3483733 |
| Sequence 10435 | AF086557 | Genbank | 3483902 |
| Sequence 10436 | AF087481 | Genbank | 4322487 |
| Sequence 10437 | AF087659 | Genbank | 4191345 |
| Sequence 10438 | AF088028 | Genbank | 3523234 |
| Sequence 10439 | AF088036 | Genbank | 3523242 |
| Sequence 10440 | AF091071 | Genbank | 3859979 |
| Sequence 10441 | AF092094 | Genbank | 4426606 |
| Sequence 10442 | AF092130 | Genbank | 5138909 |
| Sequence 10443 | AF092135 | Genbank | 5138919 |
| Sequence 10444 | AF094481 | Genbank | 4140681 |
| Sequence 10445 | AF097026 | Genbank | 6409120 |
| Sequence 10446 | AF097514 | Genbank | 4808600 |
| Sequence 10447 | AF098162 | Genbank | 3929582 |
| Sequence 10448 | AF100928 | Genbank | 4323586 |
| Sequence 10449 | AF105278 | Genbank | 4324471 |
| Sequence 10450 | AF106622 | Genbank | 4378528 |
| Sequence 10451 | AF109134 | Genbank | 4139227 |
| Sequence 10452 | AF109161 | Genbank | 4193945 |
| Sequence 10453 | AF110460 | Genbank | 4324698 |
| Sequence 10454 | AF111713 | Genbank | 5326796 |
| Sequence 10455 | AF112215 | Genbank | 6563217 |
| Sequence 10456 | AF112218 | Genbank | 6563223 |
| Sequence 10457 | AF112222 | Genbank | 6563229 |
| Sequence 10458 | AF113016 | Genbank | 6642755 |
| Sequence 10459 | AF113700 | Genbank | 6855634 |
| Sequence 10460 | AF117231 | Genbank | 6563235 |
| Sequence 10461 | AF117616 | Genbank | 4886909 |
| Sequence 10462 | AF118091 | Genbank | 6650827 |
| Sequence 10463 | AF121219 | Genbank | 4903275 |
| Sequence 10464 | AF121863 | Genbank | 4689265 |
| Sequence 10465 | AF122004 | Genbank | 5059061 |
| Sequence 10466 | AF125100 | Genbank | 5106995 |
| Sequence 10467 | AF125102 | Genbank | 5106999 |
| Sequence 10468 | AF126021 | Genbank | 6563273 |
| Sequence 10469 | AF126782 | Genbank | 6318547 |
| Sequence 10470 | AF126962 | Genbank | 6469610 |
| Sequence 10471 | AF128527 | Genbank | 4928043 |
| Sequence 10472 | AF129756 | Genbank | 4337095 |
| Sequence 10473 | AF129927 | Genbank | 4558506 |
| Sequence 10474 | AF131738 | Genbank | 4406548 |
| Sequence 10475 | AF131838 | Genbank | 4406677 |
| Sequence 10476 | AF131857 | Genbank | 4406704 |
| Sequence 10477 | AF132938 | Genbank | 4680646 |
| Sequence 10478 | AF132939 | Genbank | 4680648 |
| Sequence 10479 | AF132956 | Genbank | 4680682 |
| Sequence 10480 | AF132973 | Genbank | 4680716 |
| Sequence 10481 | AF132984 | Genbank | 4959567 |
| Sequence 10482 | AF134726 | Genbank | 4529886 |
| Sequence 10483 | AF144755 | Genbank | 5006628 |
| Sequence 10484 | AF146651 | Genbank | 5020073 |
| Sequence 10485 | AF147330 | Genbank | 4761681 |
| Sequence 10486 | AF151028 | Genbank | 7106777 |
| Sequence 10487 | AF151047 | Genbank | 7106815 |
| Sequence 10488 | AF151109 | Genbank | 6166337 |
| Sequence 10489 | AF151807 | Genbank | 4929566 |
| Sequence 10490 | AF151832 | Genbank | 4929616 |
| Sequence 10491 | AF151846 | Genbank | 4929644 |
| Sequence 10492 | AF151861 | Genbank | 4929674 |
| Sequence 10493 | AF151869 | Genbank | 4929690 |
| Sequence 10494 | AF151887 | Genbank | 4929726 |
| Sequence 10495 | AF151905 | Genbank | 4929762 |
| Sequence 10496 | AF152330 | Genbank | 5456956 |
| Sequence 10497 | AF153612 | Genbank | 4929830 |
| Sequence 10498 | AF156965 | Genbank | 5731112 |
| Sequence 10499 | AF161410 | Genbank | 6841233 |
| Sequence 10500 | AF161421 | Genbank | 6841255 |
| Sequence 10501 | AF161425 | Genbank | 6841263 |
| Sequence 10502 | AF161428 | Genbank | 6841269 |
| Sequence 10503 | AF161448 | Genbank | 6841309 |
| Sequence 10504 | AF161485 | Genbank | 6841493 |
| Sequence 10505 | AF161490 | Genbank | 6841503 |
| Sequence 10506 | AF161503 | Genbank | 6841529 |
| Sequence 10507 | AF161556 | Genbank | 6841379 |
| Sequence 10508 | AF161800 | Genbank | 5353769 |
| Sequence 10509 | AF167570 | Genbank | 5762314 |
| Sequence 10510 | AF168956 | Genbank | 5702387 |
| Sequence 10511 | AF172368 | Genbank | 5679370 |
| Sequence 10512 | AF176796 | Genbank | 5759308 |
| Sequence 10513 | AF179274 | Genbank | 5911402 |
| Sequence 10514 | AF180473 | Genbank | 6856202 |
| Sequence 10515 | AF186249 | Genbank | 6572947 |
| Sequence 10516 | AF187554 | Genbank | 6653225 |
| Sequence 10517 | AF190465 | Genbank | 6120105 |
| Sequence 10518 | AF196779 | Genbank | 6180170 |
| Sequence 10519 | AF199339 | Genbank | 6708280 |
| Sequence 10520 | AF203815 | Genbank | 6979641 |
| Sequence 10521 | AF207664 | Genbank | 6685071 |
| Sequence 10522 | AF213884 | Genbank | 7012904 |
| Sequence 10523 | AF217403 | Genbank | 6691160 |
| Sequence 10524 | AJ000519 | Genbank | 2739214 |
| Sequence 10525 | AJ000881 | Genbank | 2924308 |
| Sequence 10526 | AJ001443 | Genbank | 6006514 |
| Sequence 10527 | AJ001866 | Genbank | 2969902 |
| Sequence 10528 | AJ001981 | Genbank | 2546978 |
| Sequence 10529 | AJ002030 | Genbank | 2570006 |
| Sequence 10530 | AJ006291 | Genbank | 3805937 |
| Sequence 10531 | AJ010069 | Genbank | 3483012 |
| Sequence 10532 | AJ010597 | Genbank | 3559873 |
| Sequence 10533 | AJ011497 | Genbank | 4128014 |
| Sequence 10534 | AJ011930 | Genbank | 3859769 |
| Sequence 10535 | AJ012409 | Genbank | 3881975 |
| Sequence 10536 | AJ133812 | Genbank | 4704217 |
| Sequence 10537 | AJ223352 | Genbank | 3255996 |
| Sequence 10538 | AJ224442 | Genbank | 2911586 |
| Sequence 10539 | AJ245719 | Genbank | 6688168 |
| Sequence 10540 | AK000023 | Genbank | 7019837 |
| Sequence 10541 | AK000028 | Genbank | 7019844 |
| Sequence 10542 | AK000086 | Genbank | 7019944 |
| Sequence 10543 | AK000107 | Genbank | 7019979 |
| Sequence 10544 | AK000221 | Genbank | 7020163 |
| Sequence 10545 | AK000291 | Genbank | 7020274 |
| Sequence 10546 | AK000318 | Genbank | 7020322 |
| Sequence 10547 | AK000348 | Genbank | 7020373 |
| Sequence 10548 | AK000395 | Genbank | 7020455 |
| Sequence 10549 | AK000414 | Genbank | 7020486 |
| Sequence 10550 | AK000426 | Genbank | 7020505 |
| Sequence 10551 | AK000434 | Genbank | 7020520 |
| Sequence 10552 | AK000439 | Genbank | 7020528 |
| Sequence 10553 | AK000474 | Genbank | 7020586 |
| Sequence 10554 | AK000486 | Genbank | 7020607 |
| Sequence 10555 | AK000507 | Genbank | 7020644 |
| Sequence 10556 | AK000541 | Genbank | 7020705 |
| Sequence 10557 | AK000548 | Genbank | 7020717 |
| Sequence 10558 | AK000560 | Genbank | 7020738 |
| Sequence 10559 | AK000571 | Genbank | 7020756 |
| Sequence 10560 | AK000585 | Genbank | 7020779 |
| Sequence 10561 | AK000667 | Genbank | 7020906 |
| Sequence 10562 | AK000756 | Genbank | 7021041 |
| Sequence 10563 | AK000790 | Genbank | 7021092 |
| Sequence 10564 | AK000915 | Genbank | 7021878 |
| Sequence 10565 | AK000934 | Genbank | 7021909 |
| Sequence 10566 | AK001061 | Genbank | 7022095 |
| Sequence 10567 | AK001091 | Genbank | 7022140 |
| Sequence 10568 | AK001207 | Genbank | 7022315 |
| Sequence 10569 | AK001313 | Genbank | 7022490 |
| Sequence 10570 | AK001314 | Genbank | 7022491 |
| Sequence 10571 | AK001321 | Genbank | 7022503 |
| Sequence 10572 | AK001375 | Genbank | 7022596 |
| Sequence 10573 | AK001387 | Genbank | 7022615 |
| Sequence 10574 | AK001395 | Genbank | 7022625 |
| Sequence 10575 | AK001413 | Genbank | 7022655 |
| Sequence 10576 | AK001430 | Genbank | 7022682 |
| Sequence 10577 | AK001441 | Genbank | 7022699 |
| Sequence 10578 | AK001448 | Genbank | 7022713 |
| Sequence 10579 | AK001451 | Genbank | 7022717 |
| Sequence 10580 | AK001452 | Genbank | 7022718 |
| Sequence 10581 | AK001529 | Genbank | 7022840 |
| Sequence 10582 | AK001571 | Genbank | 7022906 |
| Sequence 10583 | AK001576 | Genbank | 7022913 |
| Sequence 10584 | AK001607 | Genbank | 7022965 |
| Sequence 10585 | AK001614 | Genbank | 7022977 |
| Sequence 10586 | AK001691 | Genbank | 7023106 |
| Sequence 10587 | AK001732 | Genbank | 7023176 |
| Sequence 10588 | AK001755 | Genbank | 7023219 |
| Sequence 10589 | AK001762 | Genbank | 7023231 |
| Sequence 10590 | AK002036 | Genbank | 7023674 |
| Sequence 10591 | AK002072 | Genbank | 7023732 |
| Sequence 10592 | AL008725 | Genbank | 2791551 |
| Sequence 10593 | AL009181 | Genbank | 2853179 |
| Sequence 10594 | AL021368 | Genbank | 3080468 |
| Sequence 10595 | AL021391 | Genbank | 4467344 |
| Sequence 10596 | AL021807 | Genbank | 9367202 |
| Sequence 10597 | AL021938 | Genbank | 3242170 |
| Sequence 10598 | AL022069 | Genbank | 3256174 |
| Sequence 10599 | AL022240 | Genbank | 4826471 |
| Sequence 10600 | AL022312 | Genbank | 4914501 |
| Sequence 10601 | AL022313 | Genbank | 4200326 |
| Sequence 10602 | AL022328 | Genbank | 5263010 |
| Sequence 10603 | AL022394 | Genbank | 9581758 |
| Sequence 10604 | AL023656 | Genbank | 5679746 |
| Sequence 10605 | AL024474 | Genbank | 3395511 |
| Sequence 10606 | AL024493 | Genbank | 3288439 |
| Sequence 10607 | AL030996 | Genbank | 3688349 |
| Sequence 10608 | AL031178 | Genbank | 3550040 |
| Sequence 10609 | AL031390 | Genbank | 5002609 |
| Sequence 10610 | AL031652 | Genbank | 9368398 |
| Sequence 10611 | AL031659 | Genbank | 9581755 |
| Sequence 10612 | AL031670 | Genbank | 4469083 |
| Sequence 10613 | AL031685 | Genbank | 9368423 |
| Sequence 10614 | AL033397 | Genbank | 4902626 |
| Sequence 10615 | AL033518 | Genbank | 5763714 |
| Sequence 10616 | AL033531 | Genbank | 6807582 |
| Sequence 10617 | AL034371 | Genbank | 4938279 |
| Sequence 10618 | AL034374 | Genbank | 4455565 |
| Sequence 10619 | AL034379 | Genbank | 5918013 |
| Sequence 10620 | AL034400 | Genbank | 4455461 |
| Sequence 10621 | AL034548 | Genbank | 7263904 |
| Sequence 10622 | AL034582 | Genbank | 5830348 |
| Sequence 10623 | AL035209 | Genbank | 4160217 |
| Sequence 10624 | AL035304 | Genbank | 4200231 |
| Sequence 10625 | AL035413 | Genbank | 6010110 |
| Sequence 10626 | AL035422 | Genbank | 5263001 |
| Sequence 10627 | AL035458 | Genbank | 6624641 |
| Sequence 10628 | AL035541 | Genbank | 9581764 |
| Sequence 10629 | AL035661 | Genbank | 6015535 |
| Sequence 10630 | AL035699 | Genbank | 4826515 |
| Sequence 10631 | AL049280 | Genbank | 4500037 |
| Sequence 10632 | AL049442 | Genbank | 4500222 |
| Sequence 10633 | AL049563 | Genbank | 4902753 |
| Sequence 10634 | AL049610 | Genbank | 5679448 |
| Sequence 10635 | AL049712 | Genbank | 5629919 |
| Sequence 10636 | AL049779 | Genbank | 8176894 |
| Sequence 10637 | AL049823 | Genbank | 6273553 |
| Sequence 10638 | AL049829 | Genbank | 8217859 |
| Sequence 10639 | AL049830 | Genbank | 6729561 |
| Sequence 10640 | AL049835 | Genbank | 5708093 |
| Sequence 10641 | AL049843 | Genbank | 5830432 |
| Sequence 10642 | AL050161 | Genbank | 4884375 |
| Sequence 10643 | AL050265 | Genbank | 4886440 |
| Sequence 10644 | AL050275 | Genbank | 4886500 |
| Sequence 10645 | AL050331 | Genbank | 5668655 |
| Sequence 10646 | AL050343 | Genbank | 6137008 |
| Sequence 10647 | AL050363 | Genbank | 4914597 |
| Sequence 10648 | AL050383 | Genbank | 4914585 |
| Sequence 10649 | AL050392 | Genbank | 4914613 |
| Sequence 10650 | AL078621 | Genbank | 6013067 |
| Sequence 10651 | AL079295 | Genbank | 5102607 |
| Sequence 10652 | AL079312 | Genbank | 5102890 |
| Sequence 10653 | AL079342 | Genbank | 6018784 |
| Sequence 10654 | AL080080 | Genbank | 5262491 |
| Sequence 10655 | AL080084 | Genbank | 5262498 |
| Sequence 10656 | AL080085 | Genbank | 5262499 |
| Sequence 10657 | AL080088 | Genbank | 5262504 |
| Sequence 10658 | AL080089 | Genbank | 5262506 |
| Sequence 10659 | AL080139 | Genbank | 5262583 |
| Sequence 10660 | AL080178 | Genbank | 5262652 |
| Sequence 10661 | AL080243 | Genbank | 5870622 |
| Sequence 10662 | AL080276 | Genbank | 5763753 |
| Sequence 10663 | AL080317 | Genbank | 5830430 |
| Sequence 10664 | AL096701 | Genbank | 5912599 |
| Sequence 10665 | AL096764 | Genbank | 7228319 |
| Sequence 10666 | AL109613 | Genbank | 6136931 |
| Sequence 10667 | AL109754 | Genbank | 6911635 |
| Sequence 10668 | AL109758 | Genbank | 8217865 |
| Sequence 10669 | AL109925 | Genbank | 7105935 |
| Sequence 10670 | AL110183 | Genbank | 5817095 |
| Sequence 10671 | AL110185 | Genbank | 5817098 |
| Sequence 10672 | AL110202 | Genbank | 5817121 |
| Sequence 10673 | AL117350 | Genbank | 7159817 |
| Sequence 10674 | AL117461 | Genbank | 5911922 |
| Sequence 10675 | AL117481 | Genbank | 5911958 |
| Sequence 10676 | AL117694 | Genbank | 8248721 |
| Sequence 10677 | AL121601 | Genbank | 7159760 |
| Sequence 10678 | AL121694 | Genbank | 6318179 |
| Sequence 10679 | AL121716 | Genbank | 7159797 |
| Sequence 10680 | AL122003 | Genbank | 6580480 |
| Sequence 10681 | AL132821 | Genbank | 7159786 |
| Sequence 10682 | AL132992 | Genbank | 6996086 |
| Sequence 10683 | AL133060 | Genbank | 6453483 |
| Sequence 10684 | AL133163 | Genbank | 6562367 |
| Sequence 10685 | AL133216 | Genbank | 6983473 |
| Sequence 10686 | AL133245 | Genbank | 7159621 |
| Sequence 10687 | AL133396 | Genbank | 6562003 |
| Sequence 10688 | AL133585 | Genbank | 6599171 |
| Sequence 10689 | AL135744 | Genbank | 6682291 |
| Sequence 10690 | AL135784 | Genbank | 6911343 |
| Sequence 10691 | AL135858 | Genbank | 7159622 |
| Sequence 10692 | AL136059 | Genbank | 7106618 |
| Sequence 10693 | AL136296 | Genbank | 8217913 |
| Sequence 10694 | AL136543 | Genbank | 6807646 |
| Sequence 10695 | AL136593 | Genbank | 7018431 |
| Sequence 10696 | AL136884 | Genbank | 6807672 |
| Sequence 10697 | AL137229 | Genbank | 7009597 |
| Sequence 10698 | AL137440 | Genbank | 6808001 |
| Sequence 10699 | AL137489 | Genbank | 6808110 |
| Sequence 10700 | AL137496 | Genbank | 6808137 |
| Sequence 10701 | AL137519 | Genbank | 6808181 |
| Sequence 10702 | AL137543 | Genbank | 6808222 |
| Sequence 10703 | AL137686 | Genbank | 6807956 |
| Sequence 10704 | AL137692 | Genbank | 6808008 |
| Sequence 10705 | AL137751 | Genbank | 6808387 |
| Sequence 10706 | AL137818 | Genbank | 7009598 |
| Sequence 10707 | AP000046 | Genbank | 3132356 |
| Sequence 10708 | AP000085 | Genbank | 4730826 |
| Sequence 10709 | AP000217 | Genbank | 4827264 |
| Sequence 10710 | AP000345 | Genbank | 5103008 |
| Sequence 10711 | AP000350 | Genbank | 5103013 |
| Sequence 10712 | AP000352 | Genbank | 6016843 |
| Sequence 10713 | AP000500 | Genbank | 5926687 |
| Sequence 10714 | AP000525 | Genbank | 5931503 |
| Sequence 10715 | AP000700 | Genbank | 6705908 |
| Sequence 10716 | AP000962 | Genbank | 6942330 |
| Sequence 10717 | AP001052 | Genbank | 6693602 |
| Sequence 10718 | AP001136 | Genbank | 6970360 |
| Sequence 10719 | AP001137 | Genbank | 6970361 |
| Sequence 10720 | AP001138 | Genbank | 7262569 |
| Sequence 10721 | AP001172 | Genbank | 6983884 |
| Sequence 10722 | AP001252 | Genbank | 7077183 |
| Sequence 10723 | AP001343 | Genbank | 7209831 |
| Sequence 10724 | AP001346 | Genbank | 7209834 |
| Sequence 10725 | D13866 | Genbank | 433410 |
| Sequence 10726 | D13900 | Genbank | 433412 |
| Sequence 10727 | D14043 | Genbank | 219924 |
| Sequence 10728 | D14658 | Genbank | 285940 |
| Sequence 10729 | D14662 | Genbank | 285948 |
| Sequence 10730 | D14665 | Genbank | 6630617 |
| Sequence 10731 | D14696 | Genbank | 285962 |
| Sequence 10732 | D14710 | Genbank | 559324 |
| Sequence 10733 | D15057 | Genbank | 493244 |
| Sequence 10734 | D16111 | Genbank | 435637 |
| Sequence 10735 | D16234 | Genbank | 303617 |
| Sequence 10736 | D16481 | Genbank | 473711 |
| Sequence 10737 | D16938 | Genbank | 598868 |
| Sequence 10738 | D16947 | Genbank | 598932 |
| Sequence 10739 | D21089 | Genbank | 475156 |
| Sequence 10740 | D25218 | Genbank | 434778 |
| Sequence 10741 | D26600 | Genbank | 565650 |
| Sequence 10742 | D29805 | Genbank | 474986 |
| Sequence 10743 | D38112 | Genbank | 644480 |
| Sequence 10744 | D42041 | Genbank | 577294 |
| Sequence 10745 | D42052 | Genbank | 1136740 |
| Sequence 10746 | D42063 | Genbank | 924266 |
| Sequence 10747 | D45915 | Genbank | 1483130 |
| Sequence 10748 | D49355 | Genbank | 1020405 |
| Sequence 10749 | D50372 | Genbank | 2605593 |
| Sequence 10750 | D50929 | Genbank | 1469200 |
| Sequence 10751 | D63486 | Genbank | 1469885 |
| Sequence 10752 | D63874 | Genbank | 968887 |
| Sequence 10753 | D66904 | Genbank | 1235726 |
| Sequence 10754 | D67025 | Genbank | 2656091 |
| Sequence 10755 | D79986 | Genbank | 1136389 |
| Sequence 10756 | D80000 | Genbank | 1136415 |
| Sequence 10757 | D80007 | Genbank | 1136429 |
| Sequence 10758 | D83253 | Genbank | 2951772 |
| Sequence 10759 | D84907 | Genbank | 1596053 |
| Sequence 10760 | D85181 | Genbank | 1906795 |
| Sequence 10761 | D86962 | Genbank | 1503997 |
| Sequence 10762 | D87018 | Genbank | 2114280 |
| Sequence 10763 | D87127 | Genbank | 1817551 |
| Sequence 10764 | D87666 | Genbank | 1620016 |
| Sequence 10765 | D87742 | Genbank | 1665824 |
| Sequence 10766 | D89052 | Genbank | 1694672 |
| Sequence 10767 | E01716 | Genbank | 2169969 |
| Sequence 10768 | E01932 | Genbank | 2170180 |
| Sequence 10769 | E01954 | Genbank | 2170202 |
| Sequence 10770 | E02628 | Genbank | 2170856 |
| Sequence 10771 | E13330 | Genbank | 3252135 |
| Sequence 10772 | J01415 | Genbank | 1944628 |
| Sequence 10773 | J03077 | Genbank | 183230 |
| Sequence 10774 | J03473 | Genbank | 337423 |
| Sequence 10775 | J03746 | Genbank | 183655 |
| Sequence 10776 | J04799 | Genbank | 190197 |
| Sequence 10777 | J04823 | Genbank | 1311703 |
| Sequence 10778 | J05016 | Genbank | 181507 |
| Sequence 10779 | J05480 | Genbank | 179807 |
| Sequence 10780 | L01057 | Genbank | 292462 |
| Sequence 10781 | L04483 | Genbank | 292444 |
| Sequence 10782 | L05092 | Genbank | 388031 |
| Sequence 10783 | L05628 | Genbank | 1835658 |
| Sequence 10784 | L08125 | Genbank | 306500 |
| Sequence 10785 | L09159 | Genbank | 307374 |
| Sequence 10786 | L11370 | Genbank | 387674 |
| Sequence 10787 | L12136 | Genbank | 181536 |
| Sequence 10788 | L12168 | Genbank | 178083 |
| Sequence 10789 | L19185 | Genbank | 440307 |
| Sequence 10790 | L20941 | Genbank | 507251 |
| Sequence 10791 | L28809 | Genbank | 454151 |
| Sequence 10792 | L29252 | Genbank | 808011 |
| Sequence 10793 | L31610 | Genbank | 1220360 |
| Sequence 10794 | L34160 | Genbank | 642091 |
| Sequence 10795 | L35681 | Genbank | 532031 |
| Sequence 10796 | L38933 | Genbank | 1008841 |
| Sequence 10797 | L38951 | Genbank | 893287 |
| Sequence 10798 | L38995 | Genbank | 704415 |
| Sequence 10799 | L39068 | Genbank | 994714 |
| Sequence 10800 | L39945 | Genbank | 703081 |
| Sequence 10801 | L40407 | Genbank | 703117 |
| Sequence 10802 | L41887 | Genbank | 950423 |
| Sequence 10803 | L47647 | Genbank | 1000861 |
| Sequence 10804 | L48984 | Genbank | 1066728 |
| Sequence 10805 | L78754 | Genbank | 1374832 |
| Sequence 10806 | M11147 | Genbank | 182513 |
| Sequence 10807 | M15887 | Genbank | 181960 |
| Sequence 10808 | M16342 | Genbank | 184266 |
| Sequence 10809 | M17886 | Genbank | 190233 |
| Sequence 10810 | M18930 | Genbank | 184371 |
| Sequence 10811 | M19989 | Genbank | 189719 |
| Sequence 10812 | M19997 | Genbank | 181968 |
| Sequence 10813 | M20496 | Genbank | 809235 |
| Sequence 10814 | M21142 | Genbank | 183402 |
| Sequence 10815 | M21154 | Genbank | 178517 |
| Sequence 10816 | M21575 | Genbank | 1311702 |
| Sequence 10817 | M24543 | Genbank | 341200 |
| Sequence 10818 | M24902 | Genbank | 189618 |
| Sequence 10819 | M25639 | Genbank | 188555 |
| Sequence 10820 | M26481 | Genbank | 619789 |
| Sequence 10821 | M26663 | Genbank | 618463 |
| Sequence 10822 | M27024 | Genbank | 341598 |
| Sequence 10823 | M29064 | Genbank | 337452 |
| Sequence 10824 | M29366 | Genbank | 181979 |
| Sequence 10825 | M29877 | Genbank | 178408 |
| Sequence 10826 | M31520 | Genbank | 337504 |
| Sequence 10827 | M33146 | Genbank | 181070 |
| Sequence 10828 | M33616 | Genbank | 184381 |
| Sequence 10829 | M34600 | Genbank | 180934 |
| Sequence 10830 | M36341 | Genbank | 178984 |
| Sequence 10831 | M37583 | Genbank | 184059 |
| Sequence 10832 | M58459 | Genbank | 337511 |
| Sequence 10833 | M58485 | Genbank | 180154 |
| Sequence 10834 | M60457 | Genbank | 181249 |
| Sequence 10835 | M60756 | Genbank | 184085 |
| Sequence 10836 | M64929 | Genbank | 190421 |
| Sequence 10837 | M69181 | Genbank | 641957 |
| Sequence 10838 | M74775 | Genbank | 187151 |
| Sequence 10839 | M76299 | Genbank | 188778 |
| Sequence 10840 | M81182 | Genbank | 190128 |
| Sequence 10841 | M81601 | Genbank | 339442 |
| Sequence 10842 | M83088 | Genbank | 189925 |
| Sequence 10843 | M93036 | Genbank | 182904 |
| Sequence 10844 | M95775 | Genbank | 187180 |
| Sequence 10845 | M96256 | Genbank | 182625 |
| Sequence 10846 | M96982 | Genbank | 338262 |
| Sequence 10847 | S48220 | Genbank | 257451 |
| Sequence 10848 | S67861 | Genbank | 239579 |
| Sequence 10849 | S70290 | Genbank | 546602 |
| Sequence 10850 | S72481 | Genbank | 632789 |
| Sequence 10851 | S75463 | Genbank | 833998 |
| Sequence 10852 | U00968 | Genbank | 409404 |
| Sequence 10853 | U01925 | Genbank | 460086 |
| Sequence 10854 | U09582 | Genbank | 493079 |
| Sequence 10855 | U09646 | Genbank | 1041191 |
| Sequence 10856 | U09813 | Genbank | 1008454 |
| Sequence 10857 | U12404 | Genbank | 531170 |
| Sequence 10858 | U12465 | Genbank | 562073 |
| Sequence 10859 | U14650 | Genbank | 541612 |
| Sequence 10860 | U14970 | Genbank | 550020 |
| Sequence 10861 | U15008 | Genbank | 600747 |
| Sequence 10862 | U16797 | Genbank | 902370 |
| Sequence 10863 | U17899 | Genbank | 717053 |
| Sequence 10864 | U19144 | Genbank | 914902 |
| Sequence 10865 | U21090 | Genbank | 1008457 |
| Sequence 10866 | U23803 | Genbank | 773643 |
| Sequence 10867 | U25182 | Genbank | 799380 |
| Sequence 10868 | U28369 | Genbank | 974283 |
| Sequence 10869 | U30826 | Genbank | 1049079 |
| Sequence 10870 | U32944 | Genbank | 1209060 |
| Sequence 10871 | U36336 | Genbank | 1209628 |
| Sequence 10872 | U38178 | Genbank | 1145301 |
| Sequence 10873 | U44754 | Genbank | 1174202 |
| Sequence 10874 | U47742 | Genbank | 1517913 |
| Sequence 10875 | U49245 | Genbank | 1216503 |
| Sequence 10876 | U49973 | Genbank | 2226003 |
| Sequence 10877 | U50939 | Genbank | 1314559 |
| Sequence 10878 | U52111 | Genbank | 8331754 |
| Sequence 10879 | U54993 | Genbank | 4097314 |
| Sequence 10880 | U55937 | Genbank | 1575044 |
| Sequence 10881 | U57847 | Genbank | 1373420 |
| Sequence 10882 | U58522 | Genbank | 1381163 |
| Sequence 10883 | U60644 | Genbank | 1575346 |
| Sequence 10884 | U60808 | Genbank | 1915971 |
| Sequence 10885 | U62961 | Genbank | 1519051 |
| Sequence 10886 | U65896 | Genbank | 1763689 |
| Sequence 10887 | U66871 | Genbank | 1519518 |
| Sequence 10888 | U68105 | Genbank | 1562509 |
| Sequence 10889 | U73824 | Genbank | 1857236 |
| Sequence 10890 | U77643 | Genbank | 2062390 |
| Sequence 10891 | U79273 | Genbank | 1710239 |
| Sequence 10892 | U79274 | Genbank | 1710240 |
| Sequence 10893 | U80017 | Genbank | 1737211 |
| Sequence 10894 | U83463 | Genbank | 1916849 |
| Sequence 10895 | U87460 | Genbank | 2076881 |
| Sequence 10896 | U90441 | Genbank | 2439984 |
| Sequence 10897 | U90915 | Genbank | 1913895 |
| Sequence 10898 | U91323 | Genbank | 3582311 |
| Sequence 10899 | U91328 | Genbank | 2088550 |
| Sequence 10900 | U97670 | Genbank | 3264860 |
| Sequence 10901 | V00478 | Genbank | 28244 |
| Sequence 10902 | X00351 | Genbank | 28251 |
| Sequence 10903 | X03083 | Genbank | 34309 |
| Sequence 10904 | X04588 | Genbank | 37423 |
| Sequence 10905 | X05332 | Genbank | 35740 |
| Sequence 10906 | X13238 | Genbank | 1200056 |
| Sequence 10907 | X15187 | Genbank | 37260 |
| Sequence 10908 | X15341 | Genbank | 1197215 |
| Sequence 10909 | X15822 | Genbank | 30146 |
| Sequence 10910 | X51525 | Genbank | 30170 |
| Sequence 10911 | X53280 | Genbank | 29504 |
| Sequence 10912 | X53586 | Genbank | 33943 |
| Sequence 10913 | X56932 | Genbank | 23690 |
| Sequence 10914 | X58288 | Genbank | 32455 |
| Sequence 10915 | X60221 | Genbank | 509290 |
| Sequence 10916 | X63432 | Genbank | 28335 |
| Sequence 10917 | X63657 | Genbank | 296185 |
| Sequence 10918 | X64707 | Genbank | 29382 |
| Sequence 10919 | X65923 | Genbank | 31302 |
| Sequence 10920 | X67247 | Genbank | 36149 |
| Sequence 10921 | X67951 | Genbank | 287640 |
| Sequence 10922 | X74801 | Genbank | 671526 |
| Sequence 10923 | X74968 | Genbank | 439659 |
| Sequence 10924 | X75821 | Genbank | 509780 |
| Sequence 10925 | X75861 | Genbank | 456258 |
| Sequence 10926 | X78136 | Genbank | 460772 |
| Sequence 10927 | X78137 | Genbank | 460770 |
| Sequence 10928 | X78669 | Genbank | 469884 |
| Sequence 10929 | X80199 | Genbank | 2385366 |
| Sequence 10930 | X81109 | Genbank | 535057 |
| Sequence 10931 | X84694 | Genbank | 872117 |
| Sequence 10932 | X84908 | Genbank | 1502344 |
| Sequence 10933 | X86779 | Genbank | 1006658 |
| Sequence 10934 | X87176 | Genbank | 1050516 |
| Sequence 10935 | X87949 | Genbank | 1143491 |
| Sequence 10936 | X91790 | Genbank | 2511667 |
| Sequence 10937 | X97544 | Genbank | 1770563 |
| Sequence 10938 | X98356 | Genbank | 1648869 |
| Sequence 10939 | X99585 | Genbank | 1770518 |
| Sequence 10940 | Y00052 | Genbank | 30308 |
| Sequence 10941 | Y00282 | Genbank | 36048 |
| Sequence 10942 | Y00345 | Genbank | 35569 |
| Sequence 10943 | Y00815 | Genbank | 34266 |
| Sequence 10944 | Y13620 | Genbank | 2181877 |
| Sequence 10945 | Y15286 | Genbank | 2584788 |
| Sequence 10946 | Z11692 | Genbank | 31107 |
| Sequence 10947 | Z14000 | Genbank | 296063 |
| Sequence 10948 | Z24724 | Genbank | 505034 |
| Sequence 10949 | Z36832 | Genbank | 533946 |
| Sequence 10950 | Z37986 | Genbank | 780262 |
| Sequence 10951 | Z37994 | Genbank | 556810 |
| Sequence 10952 | Z49250 | Genbank | 807889 |
| Sequence 10953 | Z63138 | Genbank | 1035516 |
| Sequence 10954 | Z64102 | Genbank | 1036480 |
| Sequence 10955 | Z68284 | Genbank | 1130698 |
| Sequence 10956 | Z70702 | Genbank | 1332492 |
| Sequence 10957 | Z82194 | Genbank | 2582744 |
| Sequence 10958 | Z83745 | Genbank | 1754650 |
| Sequence 10959 | Z83844 | Genbank | 4467204 |
| Sequence 10960 | Z83856 | Genbank | 2995261 |
| Sequence 10961 | Z84722 | Genbank | 1817579 |
| Sequence 10962 | Z85999 | Genbank | 2326514 |
| Sequence 10963 | Z92544 | Genbank | 1869775 |
| Sequence 10964 | Z93016 | Genbank | 9650703 |
| Sequence 10965 | Z93242 | Genbank | 3164071 |
| Sequence 10966 | Z95114 | Genbank | 5101742 |
| Sequence 10967 | Z95704 | Genbank | 2121307 |
| Sequence 10968 | Z97053 | Genbank | 9650676 |
| Sequence 10969 | Z97056 | Genbank | 2832593 |
| Sequence 10970 | Z97183 | Genbank | 2961406 |
| Sequence 10971 | Z97630 | Genbank | 4582128 |
| Sequence 10972 | Z98749 | Genbank | 4775611 |
| Sequence 10973 | Z98882 | Genbank | 4490865 |
| Sequence 10974 | Z98884 | Genbank | 5304861 |
| Sequence 10975 | AA001709 | EST | 1445523 |
| Sequence 10976 | AA004303 | EST | 1447948 |
| Sequence 10977 | AA007269 | EST | 1463303 |
| Sequence 10978 | AA010173 | EST | 1471349 |
| Sequence 10979 | AA010233 | EST | 1471260 |
| Sequence 10980 | AA010519 | EST | 1471545 |
| Sequence 10981 | AA011539 | EST | 1472565 |
| Sequence 10982 | AAO15585 | EST | 1476651 |
| Sequence 10983 | AA017170 | EST | 1479335 |
| Sequence 10984 | AA018892 | EST | 1482283 |
| Sequence 10985 | AA021623 | EST | 1485284 |
| Sequence 10986 | AA025361 | EST | 1489375 |
| Sequence 10987 | AA026455 | EST | 1492355 |
| Sequence 10988 | AA026758 | EST | 1492556 |
| Sequence 10989 | AA027906 | EST | 1493993 |
| Sequence 10990 | AA031317 | EST | 1501272 |
| Sequence 10991 | AA035377 | EST | 1507101 |
| Sequence 10992 | AA037143 | EST | 1512251 |
| Sequence 10993 | AA039734 | EST | 1516236 |
| Sequence 10994 | AA039957 | EST | 1516261 |
| Sequence 10995 | AA040714 | EST | 1516992 |
| Sequence 10996 | AA044237 | EST | 1522113 |
| Sequence 10997 | AA044575 | EST | 1522808 |
| Sequence 10998 | AA044703 | EST | 1522971 |
| Sequence 10999 | AA045239 | EST | 1523443 |
| Sequence 11000 | AA045616 | EST | 1525379 |
| Sequence 11001 | AA047046 | EST | 1524944 |
| Sequence 11002 | AA054272 | EST | 1545197 |
| Sequence 11003 | AA057541 | EST | 1550246 |
| Sequence 11004 | AA058950 | EST | 1551758 |
| Sequence 11005 | AA062994 | EST | 1557655 |
| Sequence 11006 | AA063190 | EST | 1556924 |
| Sequence 11007 | AA063198 | EST | 1556796 |
| Sequence 11008 | AA070463 | EST | 1577823 |
| Sequence 11009 | AA070785 | EST | 1578347 |
| Sequence 11010 | AA074614 | EST | 1614492 |
| Sequence 11011 | AA075478 | EST | 1615543 |
| Sequence 11012 | AA076016 | EST | 1615903 |
| Sequence 11013 | AA083482 | EST | 1625543 |
| Sequence 11014 | AA085007 | EST | 1627125 |
| Sequence 11015 | AA085810 | EST | 1629342 |
| Sequence 11016 | AA088344 | EST | 1633856 |
| Sequence 11017 | AA089630 | EST | 1636122 |
| Sequence 11018 | AA093542 | EST | 1639127 |
| Sequence 11019 | AA095108 | EST | 1640693 |
| Sequence 11020 | AA098799 | EST | 1648802 |
| Sequence 11021 | AA099464 | EST | 1645408 |
| Sequence 11022 | AA099750 | EST | 1645861 |
| Sequence 11023 | AA100034 | EST | 1647326 |
| Sequence 11024 | AA101634 | EST | 1648570 |
| Sequence 11025 | AA101663 | EST | 1648744 |
| Sequence 11026 | AA102383 | EST | 1647229 |
| Sequence 11027 | AA102507 | EST | 1647621 |
| Sequence 11028 | AA113782 | EST | 1667703 |
| Sequence 11029 | AA114830 | EST | 1669952 |
| Sequence 11030 | AA115175 | EST | 1670372 |
| Sequence 11031 | AA115455 | EST | 1670257 |
| Sequence 11032 | AA115636 | EST | 1670771 |
| Sequence 11033 | AA115738 | EST | 1670751 |
| Sequence 11034 | AA122059 | EST | 1678078 |
| Sequence 11035 | AA122097 | EST | 1678318 |
| Sequence 11036 | AA126927 | EST | 1687948 |
| Sequence 11037 | AA131505 | EST | 1692993 |
| Sequence 11038 | AA131583 | EST | 1693072 |
| Sequence 11039 | AA133311 | EST | 1690351 |
| Sequence 11040 | AA135694 | EST | 1696705 |
| Sequence 11041 | AA136084 | EST | 1697294 |
| Sequence 11042 | AA136337 | EST | 1697545 |
| Sequence 11043 | AA137210 | EST | 1697007 |
| Sequence 11044 | AA143393 | EST | 1712764 |
| Sequence 11045 | AA146684 | EST | 1716058 |
| Sequence 11046 | AA149579 | EST | 1720380 |
| Sequence 11047 | AA150361 | EST | 1721873 |
| Sequence 11048 | AA151438 | EST | 1719775 |
| Sequence 11049 | AA151565 | EST | 1720183 |
| Sequence 11050 | AA151816 | EST | 1720511 |
| Sequence 11051 | AA152016 | EST | 1720854 |
| Sequence 11052 | AA156951 | EST | 1728584 |
| Sequence 11053 | AA157731 | EST | 1729356 |
| Sequence 11054 | AA160577 | EST | 1735945 |
| Sequence 11055 | AA161189 | EST | 1735443 |
| Sequence 11056 | AA161337 | EST | 1735652 |
| Sequence 11057 | AA165446 | EST | 1741462 |
| Sequence 11058 | AA169597 | EST | 1747985 |
| Sequence 11059 | AA169657 | EST | 1748026 |
| Sequence 11060 | AA172193 | EST | 1751270 |
| Sequence 11061 | AA173705 | EST | 1753883 |
| Sequence 11062 | AA174161 | EST | 1754441 |
| Sequence 11063 | AA176485 | EST | 1757618 |
| Sequence 11064 | AA177128 | EST | 1758286 |
| Sequence 11065 | AA178986 | EST | 1760538 |
| Sequence 11066 | AA180264 | EST | 1761531 |
| Sequence 11067 | AA186328 | EST | 1774446 |
| Sequence 11068 | AA187076 | EST | 1775245 |
| Sequence 11069 | AA188297 | EST | 1775383 |
| Sequence 11070 | AA190560 | EST | 1779535 |
| Sequence 11071 | AA193327 | EST | 1782738 |
| Sequence 11072 | AA193455 | EST | 1782846 |
| Sequence 11073 | AA203319 | EST | 1799028 |
| Sequence 11074 | AA206446 | EST | 1801826 |
| Sequence 11075 | AA207077 | EST | 1802642 |
| Sequence 11076 | AA209329 | EST | 1807273 |
| Sequence 11077 | AA211900 | EST | 1810537 |
| Sequence 11078 | AA223613 | EST | 1844155 |
| Sequence 11079 | AA225958 | EST | 1847285 |
| Sequence 11080 | AA226672 | EST | 1847988 |
| Sequence 11081 | AA232991 | EST | 1856115 |
| Sequence 11082 | AA233294 | EST | 1856287 |
| Sequence 11083 | AA234637 | EST | 1859306 |
| Sequence 11084 | AA235298 | EST | 1859735 |
| Sequence 11085 | AA243646 | EST | 1874456 |
| Sequence 11086 | AA248470 | EST | 1879307 |
| Sequence 11087 | AA248684 | EST | 1879900 |
| Sequence 11088 | AA250936 | EST | 1886035 |
| Sequence 11089 | AA251061 | EST | 1886024 |
| Sequence 11090 | AA251263 | EST | 1886226 |
| Sequence 11091 | AA251692 | EST | 1886655 |
| Sequence 11092 | AA252143 | EST | 1887106 |
| Sequence 11093 | AA252608 | EST | 1887582 |
| Sequence 11094 | AA252943 | EST | 1882696 |
| Sequence 11095 | AA255735 | EST | 1892673 |
| Sequence 11096 | AA257016 | EST | 1891145 |
| Sequence 11097 | AA258732 | EST | 1893893 |
| Sequence 11098 | AA262091 | EST | 1898235 |
| Sequence 11099 | AA262425 | EST | 1897921 |
| Sequence 11100 | AA262683 | EST | 1898095 |
| Sequence 11101 | AA278796 | EST | 1920117 |
| Sequence 11102 | AA282285 | EST | 1925201 |
| Sequence 11103 | AA285126 | EST | 1928107 |
| Sequence 11104 | AA286765 | EST | 1933628 |
| Sequence 11105 | AA293822 | EST | 1941745 |
| Sequence 11106 | AA296237 | EST | 1948581 |
| Sequence 11107 | AA296690 | EST | 1949182 |
| Sequence 11108 | AA296812 | EST | 1949146 |
| Sequence 11109 | AA296923 | EST | 1949255 |
| Sequence 11110 | AA304244 | EST | 1956597 |
| Sequence 11111 | AA305213 | EST | 1957540 |
| Sequence 11112 | AA305921 | EST | 1958412 |
| Sequence 11113 | AA306323 | EST | 1958671 |
| Sequence 11114 | AA306941 | EST | 1959271 |
| Sequence 11115 | AA307728 | EST | 1960056 |
| Sequence 11116 | AA308688 | EST | 1961018 |
| Sequence 11117 | AA309119 | EST | 1961444 |
| Sequence 11118 | AA309929 | EST | 1962484 |
| Sequence 11119 | AA310375 | EST | 1962704 |
| Sequence 11120 | AA311577 | EST | 1963980 |
| Sequence 11121 | AA313294 | EST | 1965623 |
| Sequence 11122 | AA314382 | EST | 1966731 |
| Sequence 11123 | AA314635 | EST | 1967114 |
| Sequence 11124 | AA315157 | EST | 1967486 |
| Sequence 11125 | AA315989 | EST | 1968318 |
| Sequence 11126 | AA316456 | EST | 1968785 |
| Sequence 11127 | AA316676 | EST | 1969004 |
| Sequence 11128 | AA325081 | EST | 1977579 |
| Sequence 11129 | AA325713 | EST | 1977956 |
| Sequence 11130 | AA329695 | EST | 1981937 |
| Sequence 11131 | AA331249 | EST | 1983730 |
| Sequence 11132 | AA340295 | EST | 1992760 |
| Sequence 11133 | AA340744 | EST | 1992983 |
| Sequence 11134 | AA343834 | EST | 1996133 |
| Sequence 11135 | AA345527 | EST | 1997837 |
| Sequence 11136 | AA355326 | EST | 2007708 |
| Sequence 11137 | AA364646 | EST | 2016963 |
| Sequence 11138 | AA365552 | EST | 2017902 |
| Sequence 11139 | AA371377 | EST | 2023695 |
| Sequence 11140 | AA379942 | EST | 2032282 |
| Sequence 11141 | AA382161 | EST | 2034479 |
| Sequence 11142 | AA384806 | EST | 2037123 |
| Sequence 11143 | AA393525 | EST | 2046493 |
| Sequence 11144 | AA400534 | EST | 2054467 |
| Sequence 11145 | AA400644 | EST | 2054515 |
| Sequence 11146 | AA402038 | EST | 2056038 |
| Sequence 11147 | AA402743 | EST | 2057061 |
| Sequence 11148 | AA402800 | EST | 2056553 |
| Sequence 11149 | AA404235 | EST | 2058977 |
| Sequence 11150 | AA404535 | EST | 2059268 |
| Sequence 11151 | AA406310 | EST | 2064294 |
| Sequence 11152 | AA410936 | EST | 2070190 |
| Sequence 11153 | AA411736 | EST | 2069397 |
| Sequence 11154 | AA417324 | EST | 2077423 |
| Sequence 11155 | AA417743 | EST | 2079544 |
| Sequence 11156 | AA418847 | EST | 2080666 |
| Sequence 11157 | AA420643 | EST | 2094559 |
| Sequence 11158 | AA420650 | EST | 2094538 |
| Sequence 11159 | AA424006 | EST | 2102976 |
| Sequence 11160 | AA424349 | EST | 2103336 |
| Sequence 11161 | AA425535 | EST | 2106293 |
| Sequence 11162 | AA426021 | EST | 2106545 |
| Sequence 11163 | AA427560 | EST | 2111428 |
| Sequence 11164 | AA428565 | EST | 2112562 |
| Sequence 11165 | AA430605 | EST | 2111178 |
| Sequence 11166 | AA432282 | EST | 2114670 |
| Sequence 11167 | AA436100 | EST | 2141014 |
| Sequence 11168 | AA437224 | EST | 2142138 |
| Sequence 11169 | AA442070 | EST | 2153948 |
| Sequence 11170 | AA443297 | EST | 2155972 |
| Sequence 11171 | AA443762 | EST | 2156437 |
| Sequence 11172 | AA446702 | EST | 2159367 |
| Sequence 11173 | AA446759 | EST | 2159424 |
| Sequence 11174 | AA446859 | EST | 2159524 |
| Sequence 11175 | AA446942 | EST | 2159607 |
| Sequence 11176 | AA447406 | EST | 2161076 |
| Sequence 11177 | AA447871 | EST | 2161541 |
| Sequence 11178 | AA448125 | EST | 2161795 |
| Sequence 11179 | AA448292 | EST | 2161962 |
| Sequence 11180 | AA449087 | EST | 2163107 |
| Sequence 11181 | AA450129 | EST | 2163879 |
| Sequence 11182 | AA451779 | EST | 2165448 |
| Sequence 11183 | AA452120 | EST | 2165789 |
| Sequence 11184 | AA454989 | EST | 2177765 |
| Sequence 11185 | AA455869 | EST | 2178645 |
| Sequence 11186 | AA456454 | EST | 2179030 |
| Sequence 11187 | AA460766 | EST | 2185886 |
| Sequence 11188 | AA464281 | EST | 2189165 |
| Sequence 11189 | AA464409 | EST | 2189293 |
| Sequence 11190 | AA464803 | EST | 2189687 |
| Sequence 11191 | AA468360 | EST | 2194894 |
| Sequence 11192 | AA476267 | EST | 2204478 |
| Sequence 11193 | AA478669 | EST | 2207303 |
| Sequence 11194 | AA483131 | EST | 2211380 |
| Sequence 11195 | AA483251 | EST | 2212064 |
| Sequence 11196 | AA483450 | EST | 2212263 |
| Sequence 11197 | AA484155 | EST | 2212968 |
| Sequence 11198 | AA484907 | EST | 2213974 |
| Sequence 11199 | AA485065 | EST | 2214284 |
| Sequence 11200 | AA485608 | EST | 2214827 |
| Sequence 11201 | AA487694 | EST | 2217858 |
| Sequence 11202 | AA489068 | EST | 2218670 |
| Sequence 11203 | AA489782 | EST | 2220666 |
| Sequence 11204 | AA491858 | EST | 2221420 |
| Sequence 11205 | AA492143 | EST | 2221705 |
| Sequence 11206 | AA492423 | EST | 2221985 |
| Sequence 11207 | AA493522 | EST | 2223363 |
| Sequence 11208 | AA493635 | EST | 2223476 |
| Sequence 11209 | AA502361 | EST | 2237328 |
| Sequence 11210 | AA502517 | EST | 2237484 |
| Sequence 11211 | AA503033 | EST | 2238000 |
| Sequence 11212 | AA503078 | EST | 2238045 |
| Sequence 11213 | AA504572 | EST | 2240732 |
| Sequence 11214 | AA504890 | EST | 2241050 |
| Sequence 11215 | AA508394 | EST | 2245897 |
| Sequence 11216 | AA508597 | EST | 2246100 |
| Sequence 11217 | AA513123 | EST | 2251535 |
| Sequence 11218 | AA516472 | EST | 2255996 |
| Sequence 11219 | AA523172 | EST | 2263884 |
| Sequence 11220 | AA524187 | EST | 2265115 |
| Sequence 11221 | AA525960 | EST | 2268029 |
| Sequence 11222 | AA526740 | EST | 2268809 |
| Sequence 11223 | AA527181 | EST | 2269250 |
| Sequence 11224 | AA527494 | EST | 2269563 |
| Sequence 11225 | AA527667 | EST | 2269736 |
| Sequence 11226 | AA528148 | EST | 2270217 |
| Sequence 11227 | AA531585 | EST | 2274291 |
| Sequence 11228 | AA532665 | EST | 2276919 |
| Sequence 11229 | AA532805 | EST | 2278381 |
| Sequence 11230 | AA533279 | EST | 2277375 |
| Sequence 11231 | AA535098 | EST | 2279351 |
| Sequence 11232 | AA541537 | EST | 2287971 |
| Sequence 11233 | AA551141 | EST | 2321393 |
| Sequence 11234 | AA551385 | EST | 2321637 |
| Sequence 11235 | AA551709 | EST | 2321961 |
| Sequence 11236 | AA551770 | EST | 2322022 |
| Sequence 11237 | AA557174 | EST | 2327651 |
| Sequence 11238 | AA557191 | EST | 2327668 |
| Sequence 11239 | AA557633 | EST | 2328110 |
| Sequence 11240 | AA559906 | EST | 2331397 |
| Sequence 11241 | AA563778 | EST | 2335417 |
| Sequence 11242 | AA564118 | EST | 2335757 |
| Sequence 11243 | AA565432 | EST | 2337071 |
| Sequence 11244 | AA569846 | EST | 2343826 |
| Sequence 11245 | AA573721 | EST | 2348236 |
| Sequence 11246 | AA579486 | EST | 2357670 |
| Sequence 11247 | AA580489 | EST | 2355816 |
| Sequence 11248 | AA582006 | EST | 2360684 |
| Sequence 11249 | AA582252 | EST | 2359612 |
| Sequence 11250 | AA582370 | EST | 2359730 |
| Sequence 11251 | AA583922 | EST | 2368531 |
| Sequence 11252 | AA588541 | EST | 2401716 |
| Sequence 11253 | AA593402 | EST | 2409164 |
| Sequence 11254 | AA595339 | EST | 2410689 |
| Sequence 11255 | AA598710 | EST | 2432382 |
| Sequence 11256 | AA599736 | EST | 2433361 |
| Sequence 11257 | AA600194 | EST | 2433819 |
| Sequence 11258 | AA602630 | EST | 2436564 |
| Sequence 11259 | AA604339 | EST | 2445248 |
| Sequence 11260 | AA608990 | EST | 2457418 |
| Sequence 11261 | AA609462 | EST | 2457890 |
| Sequence 11262 | AA609868 | EST | 2458296 |
| Sequence 11263 | AA610424 | EST | 2458852 |
| Sequence 11264 | AA612631 | EST | 2464828 |
| Sequence 11265 | AA613033 | EST | 2464071 |
| Sequence 11266 | AA613044 | EST | 2464082 |
| Sequence 11267 | AA614081 | EST | 2466215 |
| Sequence 11268 | AA618243 | EST | 2505448 |
| Sequence 11269 | AA626635 | EST | 2539022 |
| Sequence 11270 | AA626642 | EST | 2539029 |
| Sequence 11271 | AA626833 | EST | 2539220 |
| Sequence 11272 | AA627181 | EST | 2540225 |
| Sequence 11273 | AA628451 | EST | 2540838 |
| Sequence 11274 | AA628721 | EST | 2541108 |
| Sequence 11275 | AA630616 | EST | 2553227 |
| Sequence 11276 | AA630981 | EST | 2553592 |
| Sequence 11277 | AA631024 | EST | 2553635 |
| Sequence 11278 | AA631684 | EST | 2554295 |
| Sequence 11279 | AA632534 | EST | 2555948 |
| Sequence 11280 | AA633794 | EST | 2557008 |
| Sequence 11281 | AA634087 | EST | 2557301 |
| Sequence 11282 | AA634120 | EST | 2557334 |
| Sequence 11283 | AA640721 | EST | 2565971 |
| Sequence 11284 | AA652476 | EST | 2584128 |
| Sequence 11285 | AA652829 | EST | 2584481 |
| Sequence 11286 | AA653775 | EST | 2589929 |
| Sequence 11287 | AA654914 | EST | 2591068 |
| Sequence 11288 | AA658095 | EST | 2594249 |
| Sequence 11289 | AA659440 | EST | 2595594 |
| Sequence 11290 | AA662466 | EST | 2616557 |
| Sequence 11291 | AA662530 | EST | 2616621 |
| Sequence 11292 | AA663778 | EST | 2617769 |
| Sequence 11293 | AA666385 | EST | 2620998 |
| Sequence 11294 | AA669098 | EST | 2630597 |
| Sequence 11295 | AA677560 | EST | 2658082 |
| Sequence 11296 | AA678246 | EST | 2658768 |
| Sequence 11297 | AA679533 | EST | 2660055 |
| Sequence 11298 | AA701882 | EST | 2704995 |
| Sequence 11299 | AA702803 | EST | 2705916 |
| Sequence 11300 | AA702824 | EST | 2705937 |
| Sequence 11301 | AA705800 | EST | 2715718 |
| Sequence 11302 | AA707319 | EST | 2717237 |
| Sequence 11303 | AA707321 | EST | 2717239 |
| Sequence 11304 | AA723194 | EST | 2740971 |
| Sequence 11305 | AA724307 | EST | 2742014 |
| Sequence 11306 | AA732439 | EST | 2753046 |
| Sequence 11307 | AA741013 | EST | 2779605 |
| Sequence 11308 | AA741421 | EST | 2780013 |
| Sequence 11309 | AA744747 | EST | 2783511 |
| Sequence 11310 | AA748046 | EST | 2788004 |
| Sequence 11311 | AA748621 | EST | 2788579 |
| Sequence 11312 | AA761266 | EST | 2810196 |
| Sequence 11313 | AA761352 | EST | 2810282 |
| Sequence 11314 | AA768205 | EST | 2819220 |
| Sequence 11315 | AA768918 | EST | 2820156 |
| Sequence 11316 | AA773040 | EST | 2824611 |
| Sequence 11317 | AA775624 | EST | 2834958 |
| Sequence 11318 | AA775681 | EST | 2835015 |
| Sequence 11319 | AA777245 | EST | 2836576 |
| Sequence 11320 | AA777969 | EST | 2837370 |
| Sequence 11321 | AA778560 | EST | 2837891 |
| Sequence 11322 | AA779371 | EST | 2838702 |
| Sequence 11323 | AA782583 | EST | 2841914 |
| Sequence 11324 | AA788913 | EST | 2849033 |
| Sequence 11325 | AA804448 | EST | 2875961 |
| Sequence 11326 | AA805260 | EST | 2874010 |
| Sequence 11327 | AA805691 | EST | 2874441 |
| Sequence 11328 | AA809388 | EST | 2878794 |
| Sequence 11329 | AA809587 | EST | 2878993 |
| Sequence 11330 | AA810838 | EST | 2880449 |
| Sequence 11331 | AA812263 | EST | 2881874 |
| Sequence 11332 | AA813882 | EST | 2883478 |
| Sequence 11333 | AA814716 | EST | 2884312 |
| Sequence 11334 | AA827816 | EST | 2900179 |
| Sequence 11335 | AA829667 | EST | 2902766 |
| Sequence 11336 | AA831192 | EST | 2904291 |
| Sequence 11337 | AA834216 | EST | 2907815 |
| Sequence 11338 | AA837128 | EST | 2912327 |
| Sequence 11339 | AA838535 | EST | 2913334 |
| Sequence 11340 | AA845554 | EST | 2933313 |
| Sequence 11341 | AA846156 | EST | 2932296 |
| Sequence 11342 | AA846339 | EST | 2932479 |
| Sequence 11343 | AA846735 | EST | 2932875 |
| Sequence 11344 | AA846815 | EST | 2932955 |
| Sequence 11345 | AA847840 | EST | 2934358 |
| Sequence 11346 | AA852960 | EST | 2939699 |
| Sequence 11347 | AA856586 | EST | 2944888 |
| Sequence 11348 | AA864573 | EST | 2958886 |
| Sequence 11349 | AA865331 | EST | 2957607 |
| Sequence 11350 | AA865871 | EST | 2958147 |
| Sequence 11351 | AA868273 | EST | 2963718 |
| Sequence 11352 | AA868720 | EST | 2964165 |
| Sequence 11353 | AA868840 | EST | 2964285 |
| Sequence 11354 | AA872275 | EST | 2968453 |
| Sequence 11355 | AA878211 | EST | 2987176 |
| Sequence 11356 | AA878424 | EST | 2987389 |
| Sequence 11357 | AA889466 | EST | 3016345 |
| Sequence 11358 | AA902388 | EST | 3037511 |
| Sequence 11359 | AA902974 | EST | 3038097 |
| Sequence 11360 | AA903453 | EST | 3038576 |
| Sequence 11361 | AA905040 | EST | 3040163 |
| Sequence 11362 | AA922765 | EST | 3070074 |
| Sequence 11363 | AA929066 | EST | 3078045 |
| Sequence 11364 | AA935780 | EST | 3092937 |
| Sequence 11365 | AA935796 | EST | 3092953 |
| Sequence 11366 | AA939273 | EST | 3099186 |
| Sequence 11367 | AA946596 | EST | 3109991 |
| Sequence 11368 | AA961071 | EST | 3127625 |
| Sequence 11369 | AA962465 | EST | 3134629 |
| Sequence 11370 | AA973265 | EST | 3148445 |
| Sequence 11371 | AA975592 | EST | 3151384 |
| Sequence 11372 | AA977036 | EST | 3154482 |
| Sequence 11373 | AA977991 | EST | 3155437 |
| Sequence 11374 | AA989018 | EST | 3174589 |
| Sequence 11375 | AA993218 | EST | 3179763 |
| Sequence 11376 | AA993706 | EST | 3180251 |
| Sequence 11377 | AF109301 | EST | 6782694 |
| Sequence 11378 | AI002701 | EST | 3203115 |
| Sequence 11379 | AI002736 | EST | 3203150 |
| Sequence 11380 | AI016055 | EST | 3230391 |
| Sequence 11381 | AI017469 | EST | 3231805 |
| Sequence 11382 | AI018676 | EST | 3232474 |
| Sequence 11383 | AI025274 | EST | 3240887 |
| Sequence 11384 | AI027434 | EST | 3244950 |
| Sequence 11385 | AI028115 | EST | 3245424 |
| Sequence 11386 | AI028439 | EST | 3245748 |
| Sequence 11387 | AI033712 | EST | 3254665 |
| Sequence 11388 | AI034384 | EST | 3255337 |
| Sequence 11389 | AI034428 | EST | 3255381 |
| Sequence 11390 | AI039027 | EST | 3278221 |
| Sequence 11391 | AI039136 | EST | 3278330 |
| Sequence 11392 | AI039731 | EST | 3278925 |
| Sequence 11393 | AI040803 | EST | 3279997 |
| Sequence 11394 | AI051985 | EST | 3307976 |
| Sequence 11395 | AI052598 | EST | 3308589 |
| Sequence 11396 | AI053744 | EST | 3321531 |
| Sequence 11397 | AI056319 | EST | 3330185 |
| Sequence 11398 | AI056324 | EST | 3330190 |
| Sequence 11399 | AI056337 | EST | 3330203 |
| Sequence 11400 | AI056351 | EST | 3330217 |
| Sequence 11401 | AI064691 | EST | 6358963 |
| Sequence 11402 | AI065139 | EST | 6359411 |
| Sequence 11403 | AI066536 | EST | 3367238 |
| Sequence 11404 | AI073929 | EST | 3400573 |
| Sequence 11405 | AI074143 | EST | 3400787 |
| Sequence 11406 | AI076009 | EST | 3405187 |
| Sequence 11407 | AI079555 | EST | 3415806 |
| Sequence 11408 | AI079831 | EST | 3416082 |
| Sequence 11409 | AI079885 | EST | 3416136 |
| Sequence 11410 | AI080266 | EST | 3416517 |
| Sequence 11411 | AI084035 | EST | 3422458 |
| Sequence 11412 | AI086937 | EST | 3425360 |
| Sequence 11413 | AI088270 | EST | 3427329 |
| Sequence 11414 | AI089408 | EST | 3428467 |
| Sequence 11415 | AI089953 | EST | 3429012 |
| Sequence 11416 | AI091326 | EST | 3430385 |
| Sequence 11417 | AI091580 | EST | 3430639 |
| Sequence 11418 | AI091864 | EST | 3430923 |
| Sequence 11419 | AI092638 | EST | 3431614 |
| Sequence 11420 | AI096462 | EST | 3445956 |
| Sequence 11421 | AI096678 | EST | 3446172 |
| Sequence 11422 | AI114651 | EST | 6359996 |
| Sequence 11423 | AI125541 | EST | 3594055 |
| Sequence 11424 | AI125669 | EST | 3594183 |
| Sequence 11425 | AI127461 | EST | 3595975 |
| Sequence 11426 | AI129721 | EST | 3598235 |
| Sequence 11427 | AI130978 | EST | 3600994 |
| Sequence 11428 | AI131385 | EST | 3601401 |
| Sequence 11429 | AI141972 | EST | 3649429 |
| Sequence 11430 | AI143723 | EST | 3665532 |
| Sequence 11431 | AI144216 | EST | 3666025 |
| Sequence 11432 | AI147846 | EST | 3675528 |
| Sequence 11433 | AI149616 | EST | 3678085 |
| Sequence 11434 | AI150878 | EST | 3679347 |
| Sequence 11435 | AI184622 | EST | 3735260 |
| Sequence 11436 | AI187296 | EST | 3737934 |
| Sequence 11437 | AI187413 | EST | 3738051 |
| Sequence 11438 | AI187993 | EST | 3739202 |
| Sequence 11439 | AI188768 | EST | 3739977 |
| Sequence 11440 | AI190574 | EST | 3741783 |
| Sequence 11441 | AI193729 | EST | 3744938 |
| Sequence 11442 | AI193879 | EST | 3745088 |
| Sequence 11443 | AI202702 | EST | 3755308 |
| Sequence 11444 | AI219886 | EST | 3802089 |
| Sequence 11445 | AI222521 | EST | 3804724 |
| Sequence 11446 | AI225043 | EST | 3807756 |
| Sequence 11447 | AI243691 | EST | 3839088 |
| Sequence 11448 | AI244187 | EST | 3839584 |
| Sequence 11449 | AI251103 | EST | 3847632 |
| Sequence 11450 | AI253330 | EST | 3850451 |
| Sequence 11451 | AI253436 | EST | 3850391 |
| Sequence 11452 | AI262640 | EST | 3870843 |
| Sequence 11453 | AI263331 | EST | 3871534 |
| Sequence 11454 | AI267499 | EST | 3886666 |
| Sequence 11455 | AI269682 | EST | 3888849 |
| Sequence 11456 | AI270013 | EST | 3889180 |
| Sequence 11457 | AI271339 | EST | 3890506 |
| Sequence 11458 | AI271608 | EST | 3890775 |
| Sequence 11459 | AI273048 | EST | 3895316 |
| Sequence 11460 | AI275943 | EST | 3898217 |
| Sequence 11461 | AI278640 | EST | 3916874 |
| Sequence 11462 | AI278998 | EST | 3917232 |
| Sequence 11463 | AI280181 | EST | 3918414 |
| Sequence 11464 | AI281762 | EST | 3919995 |
| Sequence 11465 | AI282246 | EST | 3920479 |
| Sequence 11466 | AI282856 | EST | 3921089 |
| Sequence 11467 | AI284545 | EST | 3922778 |
| Sequence 11468 | AI289108 | EST | 3932372 |
| Sequence 11469 | AI290847 | EST | 3933621 |
| Sequence 11470 | AI301608 | EST | 3960954 |
| Sequence 11471 | AI306464 | EST | 3989535 |
| Sequence 11472 | AI308012 | EST | 4002647 |
| Sequence 11473 | AI312384 | EST | 4017989 |
| Sequence 11474 | AI339601 | EST | 4076528 |
| Sequence 11475 | AI347153 | EST | 4084359 |
| Sequence 11476 | AI352526 | EST | 4089732 |
| Sequence 11477 | AI356383 | EST | 4108004 |
| Sequence 11478 | AI358021 | EST | 4109642 |
| Sequence 11479 | AI358557 | EST | 4110178 |
| Sequence 11480 | AI359812 | EST | 4111433 |
| Sequence 11481 | AI362921 | EST | 4114542 |
| Sequence 11482 | AI366222 | EST | 4125911 |
| Sequence 11483 | AI393262 | EST | 4222809 |
| Sequence 11484 | AI393754 | EST | 4223301 |
| Sequence 11485 | AI400359 | EST | 4243446 |
| Sequence 11486 | AI417686 | EST | 4261190 |
| Sequence 11487 | AI420543 | EST | 4266474 |
| Sequence 11488 | AI436691 | EST | 4283721 |
| Sequence 11489 | AI439350 | EST | 4303707 |
| Sequence 11490 | AI468004 | EST | 4330094 |
| Sequence 11491 | AI469347 | EST | 4331437 |
| Sequence 11492 | AI469505 | EST | 4331595 |
| Sequence 11493 | AI469600 | EST | 4331690 |
| Sequence 11494 | AI472201 | EST | 4334291 |
| Sequence 11495 | AI478202 | EST | 4371428 |
| Sequence 11496 | AI479751 | EST | 4372919 |
| Sequence 11497 | AI480256 | EST | 4373424 |
| Sequence 11498 | AI497917 | EST | 4389899 |
| Sequence 11499 | AI499665 | EST | 4391647 |
| Sequence 11500 | AI521246 | EST | 4435381 |
| Sequence 11501 | AI525607 | EST | 4439742 |
| Sequence 11502 | AI539780 | EST | 4453915 |
| Sequence 11503 | AI559444 | EST | 4509649 |
| Sequence 11504 | AI559531 | EST | 4509736 |
| Sequence 11505 | AI565555 | EST | 4524012 |
| Sequence 11506 | AI570526 | EST | 4533900 |
| Sequence 11507 | AI582754 | EST | 4568651 |
| Sequence 11508 | AI608932 | EST | 4618099 |
| Sequence 11509 | AI608936 | EST | 4618103 |
| Sequence 11510 | AI621173 | EST | 4630299 |
| Sequence 11511 | AI623863 | EST | 4648794 |
| Sequence 11512 | AI625152 | EST | 4650083 |
| Sequence 11513 | AI625819 | EST | 4650750 |
| Sequence 11514 | AI631996 | EST | 4683326 |
| Sequence 11515 | AI632955 | EST | 4684285 |
| Sequence 11516 | AI635397 | EST | 4686727 |
| Sequence 11517 | AI635756 | EST | 4687086 |
| Sequence 11518 | AI657111 | EST | 4741090 |
| Sequence 11519 | AI683799 | EST | 4893981 |
| Sequence 11520 | AI684760 | EST | 4896054 |
| Sequence 11521 | AI699114 | EST | 4987014 |
| Sequence 11522 | AI700656 | EST | 4988556 |
| Sequence 11523 | AI734838 | EST | 5056362 |
| Sequence 11524 | AI741189 | EST | 5109477 |
| Sequence 11525 | AI743974 | EST | 5112262 |
| Sequence 11526 | AI744342 | EST | 5112630 |
| Sequence 11527 | AI751021 | EST | 5129373 |
| Sequence 11528 | AI751175 | EST | 5129439 |
| Sequence 11529 | AI762495 | EST | 5178162 |
| Sequence 11530 | AI763344 | EST | 5179011 |
| Sequence 11531 | AI765690 | EST | 5232199 |
| Sequence 11532 | AI799995 | EST | 5365467 |
| Sequence 11533 | AI802542 | EST | 5368014 |
| Sequence 11534 | AI804082 | EST | 5369554 |
| Sequence 11535 | AI808503 | EST | 5395069 |
| Sequence 11536 | AI813654 | EST | 5424869 |
| Sequence 11537 | AI815499 | EST | 5431045 |
| Sequence 11538 | AI858089 | EST | 5511705 |
| Sequence 11539 | AI866603 | EST | 5530710 |
| Sequence 11540 | AI866803 | EST | 5530834 |
| Sequence 11541 | AI867464 | EST | 5540480 |
| Sequence 11542 | AI879010 | EST | 5553059 |
| Sequence 11543 | AI887361 | EST | 5592525 |
| Sequence 11544 | AI903990 | EST | 6494286 |
| Sequence 11545 | AI905002 | EST | 6495389 |
| Sequence 11546 | AI929357 | EST | 5665321 |
| Sequence 11547 | AI929483 | EST | 5665447 |
| Sequence 11548 | AI948496 | EST | 5740806 |
| Sequence 11549 | AI961342 | EST | 5754055 |
| Sequence 11550 | AI983520 | EST | 5810739 |
| Sequence 11551 | AJ133829 | EST | 4727122 |
| Sequence 11552 | AL036039 | EST | 5405665 |
| Sequence 11553 | AL036415 | EST | 5405971 |
| Sequence 11554 | AL037828 | EST | 5407161 |
| Sequence 11555 | AL039773 | EST | 5408788 |
| Sequence 11556 | AL042955 | EST | 5422386 |
| Sequence 11557 | AL043992 | EST | 5432220 |
| Sequence 11558 | AL048634 | EST | 4727722 |
| Sequence 11559 | AL119245 | EST | 5925144 |
| Sequence 11560 | AL120824 | EST | 5926825 |
| Sequence 11561 | AL134867 | EST | 6603054 |
| Sequence 11562 | AL135047 | EST | 6603234 |
| Sequence 11563 | AL138273 | EST | 6854954 |
| Sequence 11564 | AL138451 | EST | 6855132 |
| Sequence 11565 | AW003648 | EST | 5850564 |
| Sequence 11566 | AW008029 | EST | 5856807 |
| Sequence 11567 | AW008048 | EST | 5856826 |
| Sequence 11568 | AW008782 | EST | 5857560 |
| Sequence 11569 | AW021322 | EST | 5874782 |
| Sequence 11570 | AWO21588 | EST | 5875118 |
| Sequence 11571 | AW022159 | EST | 5875689 |
| Sequence 11572 | AW043631 | EST | 5904160 |
| Sequence 11573 | AW044310 | EST | 5904839 |
| Sequence 11574 | AW069285 | EST | 6024283 |
| Sequence 11575 | AW069315 | EST | 6024313 |
| Sequence 11576 | AW073580 | EST | 6028578 |
| Sequence 11577 | AW082238 | EST | 6037390 |
| Sequence 11578 | AW083099 | EST | 6038251 |
| Sequence 11579 | AW085581 | EST | 6040733 |
| Sequence 11580 | AW087200 | EST | 6043005 |
| Sequence 11581 | AW136642 | EST | 6140775 |
| Sequence 11582 | AW148690 | EST | 6196586 |
| Sequence 11583 | AW157858 | EST | 6229259 |
| Sequence 11584 | AW160706 | EST | 6299739 |
| Sequence 11585 | AW161328 | EST | 6300361 |
| Sequence 11586 | AW162943 | EST | 6301976 |
| Sequence 11587 | AW167129 | EST | 6398654 |
| Sequence 11588 | AW168050 | EST | 6399575 |
| Sequence 11589 | AW168184 | EST | 6399709 |
| Sequence 11590 | AW168400 | EST | 6399925 |
| Sequence 11591 | AW177050 | EST | 6443087 |
| Sequence 11592 | AW192594 | EST | 6471293 |
| Sequence 11593 | AW205906 | EST | 6505380 |
| Sequence 11594 | AW247236 | EST | 6590229 |
| Sequence 11595 | AW250104 | EST | 6593097 |
| Sequence 11596 | AW294606 | EST | 6701242 |
| Sequence 11597 | AW294626 | EST | 6701262 |
| Sequence 11598 | AW297943 | EST | 6704579 |
| Sequence 11599 | AW302841 | EST | 6712521 |
| Sequence 11600 | AW337419 | EST | 6834045 |
| Sequence 11601 | AW363949 | EST | 6868599 |
| Sequence 11602 | AW363975 | EST | 6868625 |
| Sequence 11603 | AW365134 | EST | 6869784 |
| Sequence 11604 | AW365960 | EST | 6870610 |
| Sequence 11605 | AW368992 | EST | 6873642 |
| Sequence 11606 | AW376614 | EST | 6881275 |
| Sequence 11607 | AW380617 | EST | 6885276 |
| Sequence 11608 | AW380631 | EST | 6885290 |
| Sequence 11609 | AW381959 | EST | 6886618 |
| Sequence 11610 | AW386920 | EST | 6891579 |
| Sequence 11611 | AW401953 | EST | 6920639 |
| Sequence 11612 | AW402499 | EST | 6921188 |
| Sequence 11613 | AW450642 | EST | 6991418 |
| Sequence 11614 | AW452921 | EST | 6993697 |
| Sequence 11615 | AW499890 | EST | 7111988 |
| Sequence 11616 | AW503119 | EST | 7118224 |
| Sequence 11617 | AW518064 | EST | N/A |
| Sequence 11618 | AW573593 | EST | 7238326 |
| Sequence 11619 | AW574740 | EST | 7246279 |
| Sequence 11620 | AW575094 | EST | 7246633 |
| Sequence 11621 | AW576694 | EST | 7251834 |
| Sequence 11622 | AW579819 | EST | 7254868 |
| Sequence 11623 | D44813 | EST | 1572288 |
| Sequence 11624 | D55269 | EST | 957166 |
| Sequence 11625 | D80421 | EST | 1178298 |
| Sequence 11626 | F07786 | EST | 677286 |
| Sequence 11627 | F20959 | EST | 2060135 |
| Sequence 11628 | F22113 | EST | 2061289 |
| Sequence 11629 | H01421 | EST | 864354 |
| Sequence 11630 | H11779 | EST | 876599 |
| Sequence 11631 | H16460 | EST | 881280 |
| Sequence 11632 | H16546 | EST | 882771 |
| Sequence 11633 | H18560 | EST | 884800 |
| Sequence 11634 | H22094 | EST | 890789 |
| Sequence 11635 | H40387 | EST | 916439 |
| Sequence 11636 | H54240 | EST | 994387 |
| Sequence 11637 | H68450 | EST | 1027190 |
| Sequence 11638 | H90819 | EST | 1081249 |
| Sequence 11639 | H94986 | EST | 1102619 |
| Sequence 11640 | L44419 | EST | 1048740 |
| Sequence 11641 | N20934 | EST | 1126104 |
| Sequence 11642 | N21624 | EST | 1126794 |
| Sequence 11643 | N23545 | EST | 1137695 |
| Sequence 11644 | N26941 | EST | 1141289 |
| Sequence 11645 | N28426 | EST | 1146662 |
| Sequence 11646 | N31770 | EST | 1152169 |
| Sequence 11647 | N40973 | EST | 1164571 |
| Sequence 11648 | N44346 | EST | 1182865 |
| Sequence 11649 | N54935 | EST | 1196255 |
| Sequence 11650 | N71715 | EST | 1228427 |
| Sequence 11651 | N80408 | EST | 1243109 |
| Sequence 11652 | R17938 | EST | 771548 |
| Sequence 11653 | R82925 | EST | 927634 |
| Sequence 11654 | R93415 | EST | 967581 |
| Sequence 11655 | R93745 | EST | 967911 |
| Sequence 11656 | T30702 | EST | 612800 |
| Sequence 11657 | T48489 | EST | 642689 |
| Sequence 11658 | T77803 | EST | 695006 |
| Sequence 11659 | T89533 | EST | 718046 |
| Sequence 11660 | W01580 | EST | 1273634 |
| Sequence 11661 | W01785 | EST | 1273784 |
| Sequence 11662 | W02490 | EST | 1274488 |
| Sequence 11663 | W33011 | EST | 1315016 |
| Sequence 11664 | W79178 | EST | 1390613 |
| Sequence 11665 | V60015 | NUCPATENT | N/A |
| Sequence 11666 | V86392 | NUCPATENT | N/A |
| Sequence 11667 | X40046 | NUCPATENT | N/A |
| Sequence 11668 | X83330 | NUCPATENT | 949933 |
| Sequence 11669 | X87396 | NUCPATENT | 1304366 |
| Sequence 11670 | X97973 | NUCPATENT | 1360128 |
| Sequence 11671 | X98409 | NUCPATENT | 1403131 |
| Sequence 11672 | Z33373 | NUCPATENT | 534033 |
| Sequence 11673 | Z33622 | NUCPATENT | 608977 |
| Sequence 11674 | Z33656 | NUCPATENT | 488829 |
| Sequence 11675 | Z39728 | NUCPATENT | 565523 |
| Sequence 11676 | AC02994 | PREPATNUC | N/A |
| Sequence 11677 | AC31122 | PREPATNUC | N/A |
| Sequence 11678 | AC34163 | PREPATNUC | N/A |
| Sequence 11679 | AC35230 | PREPATNUC | N/A |
| Sequence 11680 | AC36946 | PREPATNUC | N/A |
| Sequence 11681 | AC2759 | Genbank | 345130 |
| Sequence 11682 | A16794 | Genbank | 512417 |
| Sequence 11683 | A17003 | Genbank | 512419 |
| Sequence 11684 | A20255 | Genbank | 1247401 |
| Sequence 11685 | A36460 | Genbank | 2293778 |
| Sequence 11686 | AB000584 | Genbank | 1813326 |
| Sequence 11687 | AB001535 | Genbank | 3928755 |
| Sequence 11688 | AB002308 | Genbank | 6634012 |
| Sequence 11689 | AB002321 | Genbank | 2224586 |
| Sequence 11690 | AB002323 | Genbank | 2224590 |
| Sequence 11691 | AB002387 | Genbank | 2224718 |
| Sequence 11692 | AB002390 | Genbank | 2280487 |
| Sequence 11693 | AB002533 | Genbank | 1944124 |
| Sequence 11694 | AB003102 | Genbank | 1945608 |
| Sequence 11695 | AB003151 | Genbank | 3702678 |
| Sequence 11696 | AB003698 | Genbank | 2102636 |
| Sequence 11697 | AB003730 | Genbank | 2627128 |
| Sequence 11698 | AB007166 | Genbank | 3077761 |
| Sequence 11699 | AB007882 | Genbank | 2887418 |
| Sequence 11700 | AB007887 | Genbank | 2887428 |
| Sequence 11701 | AB007916 | Genbank | 6683704 |
| Sequence 11702 | AB007917 | Genbank | 3413857 |
| Sequence 11703 | AB007922 | Genbank | 6634036 |
| Sequence 11704 | AB007938 | Genbank | 3413899 |
| Sequence 11705 | AB010882 | Genbank | 2967451 |
| Sequence 11706 | AB011004 | Genbank | 3273315 |
| Sequence 11707 | AB011083 | Genbank | 3043545 |
| Sequence 11708 | AB011111 | Genbank | 3043601 |
| Sequence 11709 | AB011139 | Genbank | 3043657 |
| Sequence 11710 | AB011420 | Genbank | 3834353 |
| Sequence 11711 | AB014077 | Genbank | 5672586 |
| Sequence 11712 | AB014554 | Genbank | 3327121 |
| Sequence 11713 | AB015639 | Genbank | 5821139 |
| Sequence 11714 | AB017004 | Genbank | 4239949 |
| Sequence 11715 | AB017103 | Genbank | 3493334 |
| Sequence 11716 | AB017335 | Genbank | 3582440 |
| Sequence 11717 | AB017708 | Genbank | 3650434 |
| Sequence 11718 | AB018260 | Genbank | 3882154 |
| Sequence 11719 | AB018265 | Genbank | 3882164 |
| Sequence 11720 | AB018267 | Genbank | 3882168 |
| Sequence 11721 | AB018304 | Genbank | 3882242 |
| Sequence 11722 | AB018330 | Genbank | 3882294 |
| Sequence 11723 | AB018344 | Genbank | 3882322 |
| Sequence 11724 | AB019524 | Genbank | 4519939 |
| Sequence 11725 | AB019568 | Genbank | 3885371 |
| Sequence 11726 | AB020662 | Genbank | 4240198 |
| Sequence 11727 | AB020679 | Genbank | 4240232 |
| Sequence 11728 | AB020684 | Genbank | 4240242 |
| Sequence 11729 | AB020686 | Genbank | 4240246 |
| Sequence 11730 | AB020692 | Genbank | 4240258 |
| Sequence 11731 | AB020697 | Genbank | 4240268 |
| Sequence 11732 | AB020715 | Genbank | 4240304 |
| Sequence 11733 | AB020858 | Genbank | 4003378 |
| Sequence 11734 | AB020861 | Genbank | 4003381 |
| Sequence 11735 | AB021288 | Genbank | 4038732 |
| Sequence 11736 | AB021467 | Genbank | 4586518 |
| Sequence 11737 | AB021663 | Genbank | 4996450 |
| Sequence 11738 | AB022435 | Genbank | 4996607 |
| Sequence 11739 | AB022537 | Genbank | 6942217 |
| Sequence 11740 | AB023050 | Genbank | 5672605 |
| Sequence 11741 | AB023691 | Genbank | 6519222 |
| Sequence 11742 | AB023699 | Genbank | 6519298 |
| Sequence 11743 | AB024334 | Genbank | 6016837 |
| Sequence 11744 | AB026894 | Genbank | 5821423 |
| Sequence 11745 | AB027466 | Genbank | 6172220 |
| Sequence 11746 | AB028859 | Genbank | 6567165 |
| Sequence 11747 | AB029003 | Genbank | 5689496 |
| Sequence 11748 | AB029347 | Genbank | 5921132 |
| Sequence 11749 | AB029551 | Genbank | 6714542 |
| Sequence 11750 | AB032417 | Genbank | 6277265 |
| Sequence 11751 | AB032969 | Genbank | 6329965 |
| Sequence 11752 | AB032975 | Genbank | 6330044 |
| Sequence 11753 | AB033070 | Genbank | 6330818 |
| Sequence 11754 | AB033076 | Genbank | 6330860 |
| Sequence 11755 | AB033079 | Genbank | 6382025 |
| Sequence 11756 | AB033082 | Genbank | 6330910 |
| Sequence 11757 | AB033117 | Genbank | 6331435 |
| Sequence 11758 | AB034205 | Genbank | 6899845 |
| Sequence 11759 | AB037744 | Genbank | 7243026 |
| Sequence 11760 | AB037757 | Genbank | 7243052 |
| Sequence 11761 | AB037763 | Genbank | 7243064 |
| Sequence 11762 | AB037767 | Genbank | 7243072 |
| Sequence 11763 | AB037807 | Genbank | 7243152 |
| Sequence 11764 | AB037849 | Genbank | 7243236 |
| Sequence 11765 | AC000O40 | Genbank | 7923866 |
| Sequence 11766 | AC000083 | Genbank | 3810636 |
| Sequence 11767 | AC000093 | Genbank | 4678240 |
| Sequence 11768 | AC000120 | Genbank | 1809224 |
| Sequence 11769 | AC000353 | Genbank | 6970735 |
| Sequence 11770 | AC000378 | Genbank | 2270906 |
| Sequence 11771 | AC000394 | Genbank | 2133911 |
| Sequence 11772 | AC001226 | Genbank | 2133862 |
| Sequence 11773 | AC002039 | Genbank | 2342716 |
| Sequence 11774 | AC002040 | Genbank | 2347081 |
| Sequence 11775 | AC002064 | Genbank | 2076723 |
| Sequence 11776 | AC002069 | Genbank | 2076718 |
| Sequence 11777 | AC002094 | Genbank | 2155224 |
| Sequence 11778 | AC002117 | Genbank | 2281075 |
| Sequence 11779 | AC002401 | Genbank | 2599240 |
| Sequence 11780 | AC002470 | Genbank | 6478944 |
| Sequence 11781 | AC002481 | Genbank | 2340092 |
| Sequence 11782 | AC002544 | Genbank | 3337382 |
| Sequence 11783 | AC003041 | Genbank | 3264572 |
| Sequence 11784 | AC003103 | Genbank | 2842782 |
| Sequence 11785 | AC003109 | Genbank | 2961444 |
| Sequence 11786 | AC003111 | Genbank | 2636670 |
| Sequence 11787 | AC003682 | Genbank | 3264845 |
| Sequence 11788 | AC003688 | Genbank | 3789719 |
| Sequence 11789 | AC003964 | Genbank | 2981253 |
| Sequence 11790 | AC003989 | Genbank | 2772538 |
| Sequence 11791 | AC003999 | Genbank | 2772566 |
| Sequence 11792 | AC004006 | Genbank | 2781390 |
| Sequence 11793 | AC004039 | Genbank | 2811101 |
| Sequence 11794 | AC004049 | Genbank | 3366580 |
| Sequence 11795 | AC004050 | Genbank | 4001528 |
| Sequence 11796 | AC004098 | Genbank | 3097872 |
| Sequence 11797 | AC004131 | Genbank | 3342217 |
| Sequence 11798 | AC004142 | Genbank | 2880078 |
| Sequence 11799 | AC004148 | Genbank | 3482960 |
| Sequence 11800 | AC004150 | Genbank | 4585948 |
| Sequence 11801 | AC004156 | Genbank | 2896799 |
| Sequence 11802 | AC004221 | Genbank | 9665053 |
| Sequence 11803 | AC004228 | Genbank | 4263838 |
| Sequence 11804 | AC004258 | Genbank | 2924759 |
| Sequence 11805 | AC004263 | Genbank | 2935616 |
| Sequence 11806 | AC004381 | Genbank | 2982169 |
| Sequence 11807 | AC004408 | Genbank | 3873185 |
| Sequence 11808 | AC004467 | Genbank | 3132844 |
| Sequence 11809 | AC004472 | Genbank | 2984582 |
| Sequence 11810 | AC004474 | Genbank | 3097873 |
| Sequence 11811 | AC004509 | Genbank | 2996628 |
| Sequence 11812 | AC004520 | Genbank | 3004572 |
| Sequence 11813 | AC004525 | Genbank | 3219333 |
| Sequence 11814 | AC004527 | Genbank | 4760422 |
| Sequence 11815 | AC004530 | Genbank | 3108050 |
| Sequence 11816 | AC004531 | Genbank | 3337392 |
| Sequence 11817 | AC004552 | Genbank | 3273378 |
| Sequence 11818 | AC004583 | Genbank | 3293210 |
| Sequence 11819 | AC004584 | Genbank | 3417305 |
| Sequence 11820 | AC004643 | Genbank | 3097841 |
| Sequence 11821 | AC004656 | Genbank | 3253114 |
| Sequence 11822 | AC004660 | Genbank | 3108018 |
| Sequence 11823 | AC004686 | Genbank | 3688105 |
| Sequence 11824 | AC004742 | Genbank | 3152630 |
| Sequence 11825 | AC004749 | Genbank | 3157918 |
| Sequence 11826 | AC004754 | Genbank | 3165400 |
| Sequence 11827 | AC004771 | Genbank | 3668108 |
| Sequence 11828 | AC004776 | Genbank | 3169299 |
| Sequence 11829 | AC004810 | Genbank | 3264567 |
| Sequence 11830 | AC004812 | Genbank | 3970970 |
| Sequence 11831 | AC004821 | Genbank | 4753291 |
| Sequence 11832 | AC004832 | Genbank | 6624129 |
| Sequence 11833 | AC004839 | Genbank | 3983571 |
| Sequence 11834 | AC004841 | Genbank | 4454523 |
| Sequence 11835 | AC004846 | Genbank | 7243869 |
| Sequence 11836 | AC004854 | Genbank | 4827328 |
| Sequence 11837 | AC004858 | Genbank | 6624125 |
| Sequence 11838 | AC004859 | Genbank | 5091656 |
| Sequence 11839 | AC004883 | Genbank | 4263746 |
| Sequence 11840 | AC004890 | Genbank | 4508146 |
| Sequence 11841 | AC004904 | Genbank | 4156180 |
| Sequence 11842 | AC004912 | Genbank | 4156178 |
| Sequence 11843 | AC004943 | Genbank | 3924671 |
| Sequence 11844 | AC004950 | Genbank | 4753269 |
| Sequence 11845 | AC004985 | Genbank | 5708490 |
| Sequence 11846 | AC004990 | Genbank | 3924668 |
| Sequence 11847 | AC004999 | Genbank | 3970963 |
| Sequence 11848 | AC005034 | Genbank | 3947437 |
| Sequence 11849 | AC005037 | Genbank | 4827310 |
| Sequence 11850 | AC005041 | Genbank | 4508118 |
| Sequence 11851 | AC005042 | Genbank | 4156138 |
| Sequence 11852 | AC005084 | Genbank | 3659503 |
| Sequence 11853 | AC005089 | Genbank | 5732140 |
| Sequence 11854 | AC005104 | Genbank | 4218027 |
| Sequence 11855 | AC005153 | Genbank | 3242766 |
| Sequence 11856 | AC005210 | Genbank | 6249673 |
| Sequence 11857 | AC005257 | Genbank | 3289994 |
| Sequence 11858 | AC005263 | Genbank | 3289978 |
| Sequence 11859 | AC005318 | Genbank | 3885345 |
| Sequence 11860 | AC005324 | Genbank | 3366582 |
| Sequence 11861 | AC005329 | Genbank | 3342732 |
| Sequence 11862 | AC005332 | Genbank | 3659494 |
| Sequence 11863 | AC005369 | Genbank | 3367505 |
| Sequence 11864 | AC005382 | Genbank | 3386587 |
| Sequence 11865 | AC005488 | Genbank | 5836194 |
| Sequence 11866 | AC005550 | Genbank | 3478668 |
| Sequence 11867 | AC005614 | Genbank | 3540175 |
| Sequence 11868 | AC005621 | Genbank | 3548788 |
| Sequence 11869 | AC005627 | Genbank | 7243867 |
| Sequence 11870 | AC005629 | Genbank | 7243877 |
| Sequence 11871 | AC005630 | Genbank | 4159882 |
| Sequence 11872 | AC005661 | Genbank | 3818348 |
| Sequence 11873 | AC005702 | Genbank | 3688100 |
| Sequence 11874 | AC005726 | Genbank | 3810672 |
| Sequence 11875 | AC005786 | Genbank | 3702287 |
| Sequence 11876 | AC005789 | Genbank | 3702281 |
| Sequence 11877 | AC005837 | Genbank | 3849820 |
| Sequence 11878 | AC005838 | Genbank | 3947427 |
| Sequence 11879 | AC005841 | Genbank | 4731044 |
| Sequence 11880 | AC005912 | Genbank | 4165005 |
| Sequence 11881 | AC005971 | Genbank | 4314421 |
| Sequence 11882 | AC006008 | Genbank | 5091655 |
| Sequence 11883 | AC006014 | Genbank | 4699959 |
| Sequence 11884 | AC006017 | Genbank | 4508141 |
| Sequence 11885 | AC006040 | Genbank | 4314426 |
| Sequence 11886 | AC006057 | Genbank | 4731048 |
| Sequence 11887 | AC006115 | Genbank | 3962498 |
| Sequence 11888 | AC006137 | Genbank | 9887710 |
| Sequence 11889 | AC006160 | Genbank | 5701616 |
| Sequence 11890 | AC006213 | Genbank | 4160143 |
| Sequence 11891 | AC006230 | Genbank | 4966389 |
| Sequence 11892 | AC006255 | Genbank | 5306223 |
| Sequence 11893 | AC006273 | Genbank | 4096060 |
| Sequence 11894 | AC006299 | Genbank | 4106997 |
| Sequence 11895 | AC006475 | Genbank | 4753283 |
| Sequence 11896 | AC006477 | Genbank | 7243871 |
| Sequence 11897 | AC006480 | Genbank | 6289252 |
| Sequence 11898 | AC006512 | Genbank | 4926863 |
| Sequence 11899 | AC006566 | Genbank | 4309780 |
| Sequence 11900 | AC006581 | Genbank | 4914350 |
| Sequence 11901 | AC007032 | Genbank | 5523832 |
| Sequence 11902 | AC007041 | Genbank | 5708471 |
| Sequence 11903 | AC007055 | Genbank | 4885691 |
| Sequence 11904 | AC007066 | Genbank | 4508098 |
| Sequence 11905 | AC007073 | Genbank | 4416547 |
| Sequence 11906 | AC007172 | Genbank | 4731066 |
| Sequence 11907 | AC007191 | Genbank | 4558643 |
| Sequence 11908 | AC007225 | Genbank | 6715703 |
| Sequence 11909 | AC007253 | Genbank | 5649375 |
| Sequence 11910 | AC007384 | Genbank | 5708473 |
| Sequence 11911 | AC007406 | Genbank | 4689442 |
| Sequence 11912 | AC007528 | Genbank | 5263309 |
| Sequence 11913 | AC007537 | Genbank | 4914348 |
| Sequence 11914 | AC007541 | Genbank | 4982536 |
| Sequence 11915 | AC007564 | Genbank | 4982534 |
| Sequence 11916 | AC007565 | Genbank | 4835751 |
| Sequence 11917 | AC007736 | Genbank | 5732177 |
| Sequence 11918 | AC007938 | Genbank | 5306288 |
| Sequence 11919 | AC007999 | Genbank | 7007656 |
| Sequence 11920 | AC008044 | Genbank | 6015188 |
| Sequence 11921 | AC008101 | Genbank | 6970743 |
| Sequence 11922 | AC008102 | Genbank | 9797815 |
| Sequence 11923 | AC008122 | Genbank | 5931373 |
| Sequence 11924 | AC008123 | Genbank | 6006046 |
| Sequence 11925 | AC008269 | Genbank | 7243890 |
| Sequence 11926 | AC008498 | Genbank | 6850299 |
| Sequence 11927 | AC008893 | Genbank | 7259691 |
| Sequence 11928 | AC009330 | Genbank | 6553960 |
| Sequence 11929 | AC010206 | Genbank | 6539154 |
| Sequence 11930 | AC010582 | Genbank | 6721135 |
| Sequence 11931 | AC011331 | Genbank | 9929686 |
| Sequence 11932 | AC012087 | Genbank | 7114465 |
| Sequence 11933 | AC020663 | Genbank | 6682593 |
| Sequence 11934 | AC021049 | Genbank | 7248920 |
| Sequence 11935 | AF001549 | Genbank | 3355302 |
| Sequence 11936 | AF001977 | Genbank | 2914738 |
| Sequence 11937 | AF002668 | Genbank | 2232173 |
| Sequence 11938 | AF003837 | Genbank | 2228792 |
| Sequence 11939 | AF004162 | Genbank | 3046385 |
| Sequence 11940 | AF004339 | Genbank | 2245559 |
| Sequence 11941 | AF004341 | Genbank | 2245563 |
| Sequence 11942 | AF004342 | Genbank | 2245565 |
| Sequence 11943 | AF005896 | Genbank | 2209244 |
| Sequence 11944 | AF006083 | Genbank | 2282031 |
| Sequence 11945 | AF007544 | Genbank | 2970122 |
| Sequence 11946 | AF007835 | Genbank | 4102034 |
| Sequence 11947 | AF008303 | Genbank | 2580619 |
| Sequence 11948 | AF011889 | Genbank | 2335186 |
| Sequence 11949 | AF012023 | Genbank | 2305237 |
| Sequence 11950 | AF012072 | Genbank | 9967556 |
| Sequence 11951 | AF012126 | Genbank | 2832227 |
| Sequence 11952 | AF015812 | Genbank | 2599359 |
| Sequence 11953 | AF016709 | Genbank | 2731560 |
| Sequence 11954 | AF019413 | Genbank | 2347130 |
| Sequence 11955 | AF020057 | Genbank | 3150436 |
| Sequence 11956 | AF020351 | Genbank | 2655052 |
| Sequence 11957 | AF020352 | Genbank | 2655054 |
| Sequence 11958 | AF021819 | Genbank | 2460317 |
| Sequence 11959 | AF022913 | Genbank | 2558890 |
| Sequence 11960 | AF026292 | Genbank | 2559009 |
| Sequence 11961 | AF026816 | Genbank | 2583078 |
| Sequence 11962 | AF028832 | Genbank | 3287488 |
| Sequence 11963 | AF030186 | Genbank | 3831546 |
| Sequence 11964 | AF032387 | Genbank | 2641556 |
| Sequence 11965 | AF033095 | Genbank | 2645728 |
| Sequence 11966 | AF035840 | Genbank | 3800739 |
| Sequence 11967 | AF036715 | Genbank | 4007385 |
| Sequence 11968 | AF038178 | Genbank | 2795898 |
| Sequence 11969 | AF038200 | Genbank | 2795921 |
| Sequence 11970 | AF038554 | Genbank | 2895558 |
| Sequence 11971 | AF038633 | Genbank | 3252876 |
| Sequence 11972 | AF038955 | Genbank | 3329379 |
| Sequence 11973 | AF041483 | Genbank | 3493528 |
| Sequence 11974 | AF042166 | Genbank | 3298596 |
| Sequence 11975 | AF042836 | Genbank | 3776241 |
| Sequence 11976 | AF044957 | Genbank | 4164445 |
| Sequence 11977 | AF044958 | Genbank | 4164447 |
| Sequence 11978 | AF047185 | Genbank | 2909861 |
| Sequence 11979 | AF047440 | Genbank | 3335135 |
| Sequence 11980 | AF047472 | Genbank | 2921872 |
| Sequence 11981 | AF049105 | Genbank | 2984656 |
| Sequence 11982 | AF051316 | Genbank | 6580759 |
| Sequence 11983 | AF051894 | Genbank | 3095110 |
| Sequence 11984 | AF052129 | Genbank | 3360438 |
| Sequence 11985 | AF052642 | Genbank | 2981453 |
| Sequence 11986 | AF053356 | Genbank | 3135305 |
| Sequence 11987 | AF054984 | Genbank | 3005692 |
| Sequence 11988 | AF054990 | Genbank | 3005703 |
| Sequence 11989 | AF055473 | Genbank | nil |
| Sequence 11990 | AF055474 | Genbank | nil |
| Sequence 11991 | AF061737 | Genbank | 4335938 |
| Sequence 11992 | AF064019 | Genbank | 3347856 |
| Sequence 11993 | AF064084 | Genbank | 3135668 |
| Sequence 11994 | AF064245 | Genbank | 3930528 |
| Sequence 11995 | AF064861 | Genbank | 3171154 |
| Sequence 11996 | AF067517 | Genbank | 3850313 |
| Sequence 11997 | AF067844 | Genbank | 4240386 |
| Sequence 11998 | AF068754 | Genbank | 3283408 |
| Sequence 11999 | AF069378 | Genbank | 3982733 |
| Sequence 12000 | AF070646 | Genbank | 3283920 |
| Sequence 12001 | AF070649 | Genbank | 3283923 |
| Sequence 12002 | AF070652 | Genbank | 4454679 |
| Sequence 12003 | AF070655 | Genbank | 4454685 |
| Sequence 12004 | AF073792 | Genbank | 3300101 |
| Sequence 12005 | AF075053 | Genbank | 3377594 |
| Sequence 12006 | AF075242 | Genbank | 4377992 |
| Sequence 12007 | AF077042 | Genbank | 4689131 |
| Sequence 12008 | AF077043 | Genbank | 4689133 |
| Sequence 12009 | AF077046 | Genbank | 4689139 |
| Sequence 12010 | AF077052 | Genbank | 4689151 |
| Sequence 12011 | AF077202 | Genbank | 4679017 |
| Sequence 12012 | AF078855 | Genbank | 5531824 |
| Sequence 12013 | AF081282 | Genbank | 4336324 |
| Sequence 12014 | AF083441 | Genbank | 5813822 |
| Sequence 12015 | AF085361 | Genbank | 5114056 |
| Sequence 12016 | AF086168 | Genbank | 3483513 |
| Sequence 12017 | AF086169 | Genbank | 3483514 |
| Sequence 12018 | AF086210 | Genbank | 3483555 |
| Sequence 12019 | AF086236 | Genbank | 3483581 |
| Sequence 12020 | AF086329 | Genbank | 3483674 |
| Sequence 12021 | AF086465 | Genbank | 3483810 |
| Sequence 12022 | AF086557 | Genbank | 3483902 |
| Sequence 12023 | AF091075 | Genbank | 3859987 |
| Sequence 12024 | AF091083 | Genbank | 3860003 |
| Sequence 12025 | AF091084 | Genbank | 3860005 |
| Sequence 12026 | AF092124 | Genbank | 3659900 |
| Sequence 12027 | AF092135 | Genbank | 5138919 |
| Sequence 12028 | AF098162 | Genbank | 3929582 |
| Sequence 12029 | AF098865 | Genbank | 4204674 |
| Sequence 12030 | AF100748 | Genbank | 5410281 |
| Sequence 12031 | AF100928 | Genbank | 4323586 |
| Sequence 12032 | AF103801 | Genbank | 6048969 |
| Sequence 12033 | AF105278 | Genbank | 4324471 |
| Sequence 12034 | AF107893 | Genbank | 4008128 |
| Sequence 12035 | AF109872 | Genbank | 4028621 |
| Sequence 12036 | AF110304 | Genbank | 7106722 |
| Sequence 12037 | AF110460 | Genbank | 4324698 |
| Sequence 12038 | AF110774 | Genbank | 6523794 |
| Sequence 12039 | AF111847 | Genbank | 7211441 |
| Sequence 12040 | AF112215 | Genbank | 6563217 |
| Sequence 12041 | AF112222 | Genbank | 6563229 |
| Sequence 12042 | AF113016 | Genbank | 6642755 |
| Sequence 12043 | AF113680 | Genbank | 6855605 |
| Sequence 12044 | AF113700 | Genbank | 6855634 |
| Sequence 12045 | AF117235 | Genbank | 6563243 |
| Sequence 12046 | AF117829 | Genbank | 4151947 |
| Sequence 12047 | AF118091 | Genbank | 6650827 |
| Sequence 12048 | AF119663 | Genbank | 6563251 |
| Sequence 12049 | AF121219 | Genbank | 4903275 |
| Sequence 12050 | AF122004 | Genbank | 5059061 |
| Sequence 12051 | AF128536 | Genbank | 5305705 |
| Sequence 12052 | AF129927 | Genbank | 4558506 |
| Sequence 12053 | AF130249 | Genbank | 4455134 |
| Sequence 12054 | AF131738 | Genbank | 4406548 |
| Sequence 12055 | AF131838 | Genbank | 4406677 |
| Sequence 12056 | AF131856 | Genbank | 4406702 |
| Sequence 12057 | AF132965 | Genbank | 4680700 |
| Sequence 12058 | AF132973 | Genbank | 4680716 |
| Sequence 12059 | AF139065 | Genbank | 6635330 |
| Sequence 12060 | AF144700 | Genbank | 5107075 |
| Sequence 12061 | AF144755 | Genbank | 5006628 |
| Sequence 12062 | AF146191 | Genbank | 5678818 |
| Sequence 12063 | AF147330 | Genbank | 4761681 |
| Sequence 12064 | AF150089 | Genbank | 5107165 |
| Sequence 12065 | AF151020 | Genbank | 7106761 |
| Sequence 12066 | AF151066 | Genbank | 7106853 |
| Sequence 12067 | AF151109 | Genbank | 6166337 |
| Sequence 12068 | AF151807 | Genbank | 4929566 |
| Sequence 12069 | AF151832 | Genbank | 4929616 |
| Sequence 12070 | AF151841 | Genbank | 4929634 |
| Sequence 12071 | AF151861 | Genbank | 4929674 |
| Sequence 12072 | AF151875 | Genbank | 4929702 |
| Sequence 12073 | AF151879 | Genbank | 4929710 |
| Sequence 12074 | AF151884 | Genbank | 4929720 |
| Sequence 12075 | AF151886 | Genbank | 4929724 |
| Sequence 12076 | AF151889 | Genbank | 4929730 |
| Sequence 12077 | AF151893 | Genbank | 4929738 |
| Sequence 12078 | AF151905 | Genbank | 4929762 |
| Sequence 12079 | AF151906 | Genbank | 4929764 |
| Sequence 12080 | AF152462 | Genbank | 5565976 |
| Sequence 12081 | AF155568 | Genbank | 5031511 |
| Sequence 12082 | AF156965 | Genbank | 5731112 |
| Sequence 12083 | AF161389 | Genbank | 6841191 |
| Sequence 12084 | AF161448 | Genbank | 6841309 |
| Sequence 12085 | AF161466 | Genbank | 6841455 |
| Sequence 12086 | AF161500 | Genbank | 6841523 |
| Sequence 12087 | AF161503 | Genbank | 6841529 |
| Sequence 12088 | AF161512 | Genbank | 6841547 |
| Sequence 12089 | AF161556 | Genbank | 6841379 |
| Sequence 12090 | AF165926 | Genbank | 5817858 |
| Sequence 12091 | AF168787 | Genbank | 7239175 |
| Sequence 12092 | AF168956 | Genbank | 5702387 |
| Sequence 12093 | AF174496 | Genbank | 7108914 |
| Sequence 12094 | AF176574 | Genbank | 5762481 |
| Sequence 12095 | AF179274 | Genbank | 5911402 |
| Sequence 12096 | AF187320 | Genbank | 6164847 |
| Sequence 12097 | AF187554 | Genbank | 6653225 |
| Sequence 12098 | AF188178 | Genbank | 6164864 |
| Sequence 12099 | AF188745 | Genbank | 6425043 |
| Sequence 12100 | AF191018 | Genbank | 6457339 |
| Sequence 12101 | AF191298 | Genbank | 7656642 |
| Sequence 12102 | AF196969 | Genbank | 6289073 |
| Sequence 12103 | AF2O1077 | Genbank | 6456748 |
| Sequence 12104 | AF203815 | Genbank | 6979641 |
| Sequence 12105 | AF205588 | Genbank | 6531675 |
| Sequence 12106 | AF205600 | Genbank | 6581094 |
| Sequence 12107 | AF207550 | Genbank | 6470333 |
| Sequence 12108 | AF217403 | Genbank | 6691160 |
| Sequence 12109 | AF224669 | Genbank | 7012905 |
| Sequence 12110 | AJ001981 | Genbank | 2546978 |
| Sequence 12111 | AJ010069 | Genbank | 3483012 |
| Sequence 12112 | AJ010341 | Genbank | 4490794 |
| Sequence 12113 | AJ011803 | Genbank | 4753760 |
| Sequence 12114 | AJ012499 | Genbank | 5441359 |
| Sequence 12115 | AJ131693 | Genbank | 4584422 |
| Sequence 12116 | AJ224442 | Genbank | 2911586 |
| Sequence 12117 | AJ243669 | Genbank | 6688156 |
| Sequence 12118 | AJ249731 | Genbank | 5921469 |
| Sequence 12119 | AJ250042 | Genbank | 6013005 |
| Sequence 12120 | AJ250915 | Genbank | 6996445 |
| Sequence 12121 | AK000049 | Genbank | 7019880 |
| Sequence 12122 | AK000052 | Genbank | 7019885 |
| Sequence 12123 | AK000068 | Genbank | 7019914 |
| Sequence 12124 | AK000083 | Genbank | 7019940 |
| Sequence 12125 | AK000171 | Genbank | 7020084 |
| Sequence 12126 | AK000221 | Genbank | 7020163 |
| Sequence 12127 | AK000303 | Genbank | 7020295 |
| Sequence 12128 | AK000319 | Genbank | 7020324 |
| Sequence 12129 | AK000395 | Genbank | 7020455 |
| Sequence 12130 | AK000400 | Genbank | 7020463 |
| Sequence 12131 | AK000434 | Genbank | 7020520 |
| Sequence 12132 | AK000462 | Genbank | 7020566 |
| Sequence 12133 | AK000470 | Genbank | 7020580 |
| Sequence 12134 | AK000526 | Genbank | 7020680 |
| Sequence 12135 | AK000560 | Genbank | 7020738 |
| Sequence 12136 | AK000615 | Genbank | 7020827 |
| Sequence 12137 | AK000620 | Genbank | 7020834 |
| Sequence 12138 | AK000624 | Genbank | 7020839 |
| Sequence 12139 | AK000656 | Genbank | 7020889 |
| Sequence 12140 | AK000667 | Genbank | 7020906 |
| Sequence 12141 | AK000669 | Genbank | 7020909 |
| Sequence 12142 | AK000818 | Genbank | 7021128 |
| Sequence 12143 | AK000931 | Genbank | 7021906 |
| Sequence 12144 | AK001067 | Genbank | 7022104 |
| Sequence 12145 | AK001088 | Genbank | 7022136 |
| Sequence 12146 | AK001163 | Genbank | 7022244 |
| Sequence 12147 | AK001192 | Genbank | 7022292 |
| Sequence 12148 | AK001221 | Genbank | 7022340 |
| Sequence 12149 | AK001321 | Genbank | 7022503 |
| Sequence 12150 | AK001338 | Genbank | 7022533 |
| Sequence 12151 | AK001354 | Genbank | 7022561 |
| Sequence 12152 | AK001408 | Genbank | 7022645 |
| Sequence 12153 | AK001441 | Genbank | 7022699 |
| Sequence 12154 | AK001453 | Genbank | 7022720 |
| Sequence 12155 | AK001489 | Genbank | 7022777 |
| Sequence 12156 | AK001510 | Genbank | 7022808 |
| Sequence 12157 | AK001536 | Genbank | 7022851 |
| Sequence 12158 | AK001564 | Genbank | 7022893 |
| Sequence 12159 | AK001614 | Genbank | 7022977 |
| Sequence 12160 | AK001689 | Genbank | 7023104 |
| Sequence 12161 | AK001718 | Genbank | 7023153 |
| Sequence 12162 | AK001764 | Genbank | 7023235 |
| Sequence 12163 | AK001780 | Genbank | 7023265 |
| Sequence 12164 | AK001902 | Genbank | 7023455 |
| Sequence 12165 | AK002031 | Genbank | 7023667 |
| Sequence 12166 | AK002100 | Genbank | 7023777 |
| Sequence 12167 | AL008721 | Genbank | 3171883 |
| Sequence 12168 | AL008723 | Genbank | 5101762 |
| Sequence 12169 | AL009179 | Genbank | 3217024 |
| Sequence 12170 | AL020993 | Genbank | 3980107 |
| Sequence 12171 | AL020997 | Genbank | 2804155 |
| Sequence 12172 | AL021368 | Genbank | 3080468 |
| Sequence 12173 | AL021397 | Genbank | 3319943 |
| Sequence 12174 | AL021707 | Genbank | 4582132 |
| Sequence 12175 | AL021917 | Genbank | 9367203 |
| Sequence 12176 | AL022153 | Genbank | 3319674 |
| Sequence 12177 | AL022238 | Genbank | 4176442 |
| Sequence 12178 | AL022240 | Genbank | 4826471 |
| Sequence 12179 | AL022313 | Genbank | 4200326 |
| Sequence 12180 | AL022326 | Genbank | 3550039 |
| Sequence 12181 | AL022718 | Genbank | 3763969 |
| Sequence 12182 | AL022725 | Genbank | 5679748 |
| Sequence 12183 | AL023581 | Genbank | 3445283 |
| Sequence 12184 | AL023582 | Genbank | 3805923 |
| Sequence 12185 | AL023803 | Genbank | 9408726 |
| Sequence 12186 | AL023807 | Genbank | 5679750 |
| Sequence 12187 | AL024474 | Genbank | 3395511 |
| Sequence 12188 | AL031005 | Genbank | 3287156 |
| Sequence 12189 | AL031256 | Genbank | 3738133 |
| Sequence 12190 | AL031274 | Genbank | 3421083 |
| Sequence 12191 | AL031295 | Genbank | 4376011 |
| Sequence 12192 | AL031297 | Genbank | 4902714 |
| Sequence 12193 | AL031427 | Genbank | 4835258 |
| Sequence 12194 | AL031432 | Genbank | 4375969 |
| Sequence 12195 | AL031665 | Genbank | 4826504 |
| Sequence 12196 | AL031681 | Genbank | 9408729 |
| Sequence 12197 | AL031718 | Genbank | 5805110 |
| Sequence 12198 | AL031774 | Genbank | 3881948 |
| Sequence 12199 | AL033377 | Genbank | 4826462 |
| Sequence 12200 | AL033397 | Genbank | 4902626 |
| Sequence 12201 | AL033517 | Genbank | 4140315 |
| Sequence 12202 | AL033522 | Genbank | 4191258 |
| Sequence 12203 | AL033531 | Genbank | 6807582 |
| Sequence 12204 | AL034548 | Genbank | 7263904 |
| Sequence 12205 | AL035209 | Genbank | 4160217 |
| Sequence 12206 | AL035249 | Genbank | 4688857 |
| Sequence 12207 | AL035398 | Genbank | 5262352 |
| Sequence 12208 | AL035411 | Genbank | 6136940 |
| Sequence 12209 | AL035413 | Genbank | 6010110 |
| Sequence 12210 | AL035454 | Genbank | 9368364 |
| Sequence 12211 | AL035458 | Genbank | 6624641 |
| Sequence 12212 | AL035461 | Genbank | 5123778 |
| Sequence 12213 | AL035541 | Genbank | 9581764 |
| Sequence 12214 | AL035610 | Genbank | 6249454 |
| Sequence 12215 | AL035699 | Genbank | 4826515 |
| Sequence 12216 | AL049280 | Genbank | 4500037 |
| Sequence 12217 | AL049294 | Genbank | 4500055 |
| Sequence 12218 | AL049447 | Genbank | 4500230 |
| Sequence 12219 | AL049461 | Genbank | 4500250 |
| Sequence 12220 | AL049552 | Genbank | 6010194 |
| Sequence 12221 | AL049564 | Genbank | 4902757 |
| Sequence 12222 | AL049646 | Genbank | 9368462 |
| Sequence 12223 | AL049694 | Genbank | 5650653 |
| Sequence 12224 | AL049748 | Genbank | 6572235 |
| Sequence 12225 | AL049766 | Genbank | 5763746 |
| Sequence 12226 | AL049779 | Genbank | 8176894 |
| Sequence 12227 | AL049829 | Genbank | 8217859 |
| Sequence 12228 | AL049844 | Genbank | 5924019 |
| Sequence 12229 | AL049849 | Genbank | 4826529 |
| Sequence 12230 | AL049869 | Genbank | 8217861 |
| Sequence 12231 | AL049870 | Genbank | 7248268 |
| Sequence 12232 | AL049911 | Genbank | 6114591 |
| Sequence 12233 | AL049944 | Genbank | 4884189 |
| Sequence 12234 | AL049962 | Genbank | 4884212 |
| Sequence 12235 | AL049964 | Genbank | 4884214 |
| Sequence 12236 | AL049968 | Genbank | 4884217 |
| Sequence 12237 | AL050016 | Genbank | 4884262 |
| Sequence 12238 | AL050089 | Genbank | 4884107 |
| Sequence 12239 | AL050275 | Genbank | 4886500 |
| Sequence 12240 | AL050277 | Genbank | 4886454 |
| Sequence 12241 | AL050343 | Genbank | 6137008 |
| Sequence 12242 | AL078463 | Genbank | 5777575 |
| Sequence 12243 | AL078621 | Genbank | 6013067 |
| Sequence 12244 | AL079333 | Genbank | 5738434 |
| Sequence 12245 | AL080080 | Genbank | 5262491 |
| Sequence 12246 | AL080102 | Genbank | 5262526 |
| Sequence 12247 | AL080193 | Genbank | 5262674 |
| Sequence 12248 | AL080276 | Genbank | 5763753 |
| Sequence 12249 | AL109672 | Genbank | 5689836 |
| Sequence 12250 | AL109759 | Genbank | 8176900 |
| Sequence 12251 | AL109798 | Genbank | 6453386 |
| Sequence 12252 | AL109865 | Genbank | 6911646 |
| Sequence 12253 | AL110183 | Genbank | 5817095 |
| Sequence 12254 | AL110185 | Genbank | 5817098 |
| Sequence 12255 | AL110193 | Genbank | 5817109 |
| Sequence 12256 | AL110194 | Genbank | 5817111 |
| Sequence 12257 | AL110202 | Genbank | 5817121 |
| Sequence 12258 | AL110271 | Genbank | 5817083 |
| Sequence 12259 | AL117350 | Genbank | 7159817 |
| Sequence 12260 | AL117450 | Genbank | 5911900 |
| Sequence 12261 | AL117461 | Genbank | 5911922 |
| Sequence 12262 | AL117482 | Genbank | 5911960 |
| Sequence 12263 | AL117648 | Genbank | 5912240 |
| Sequence 12264 | AL117662 | Genbank | 5912257 |
| Sequence 12265 | AL121603 | Genbank | 6434634 |
| Sequence 12266 | AL121652 | Genbank | 7159615 |
| Sequence 12267 | AL121716 | Genbank | 7159797 |
| Sequence 12268 | AL121790 | Genbank | 8919824 |
| Sequence 12269 | AL121823 | Genbank | 6635879 |
| Sequence 12270 | AL121852 | Genbank | 8176919 |
| Sequence 12271 | AL121963 | Genbank | 7161783 |
| Sequence 12272 | AL122036 | Genbank | 6093222 |
| Sequence 12273 | AL122066 | Genbank | 6102860 |
| Sequence 12274 | AL133010 | Genbank | 6453416 |
| Sequence 12275 | AL133060 | Genbank | 6453483 |
| Sequence 12276 | AL133069 | Genbank | 6453507 |
| Sequence 12277 | AL133215 | Genbank | 7228177 |
| Sequence 12278 | AL133216 | Genbank | 6983473 |
| Sequence 12279 | AL133387 | Genbank | 7076389 |
| Sequence 12280 | AL133453 | Genbank | 7009594 |
| Sequence 12281 | AL133593 | Genbank | 6599181 |
| Sequence 12282 | AL133662 | Genbank | 6599256 |
| Sequence 12283 | AL135744 | Genbank | 6682291 |
| Sequence 12284 | AL135959 | Genbank | 6635874 |
| Sequence 12285 | AL136296 | Genbank | 8217913 |
| Sequence 12286 | AL136504 | Genbank | 7018294 |
| Sequence 12287 | AL136831 | Genbank | 6807666 |
| Sequence 12288 | AL137269 | Genbank | 6807703 |
| Sequence 12289 | AL137450 | Genbank | 6808024 |
| Sequence 12290 | AL137454 | Genbank | 6808031 |
| Sequence 12291 | AL137481 | Genbank | 6808096 |
| Sequence 12292 | AL137542 | Genbank | 6808221 |
| Sequence 12293 | AL137673 | Genbank | 6807841 |
| Sequence 12294 | AL137714 | Genbank | 6808128 |
| Sequence 12295 | AL137721 | Genbank | 6808159 |
| Sequence 12296 | AL137723 | Genbank | 6808203 |
| Sequence 12297 | AL137818 | Genbank | 7009598 |
| Sequence 12298 | AL157442 | Genbank | 7018558 |
| Sequence 12299 | AL157468 | Genbank | 7018484 |
| Sequence 12300 | AL157493 | Genbank | 7018539 |
| Sequence 12301 | AL158493 | Genbank | 7161641 |
| Sequence 12302 | AL158547 | Genbank | 7161742 |
| Sequence 12303 | AP000008 | Genbank | 3132318 |
| Sequence 12304 | AP000009 | Genbank | 4666255 |
| Sequence 12305 | AP000037 | Genbank | 3132347 |
| Sequence 12306 | AP000038 | Genbank | 3132348 |
| Sequence 12307 | AP000346 | Genbank | 5103009 |
| Sequence 12308 | AP000347 | Genbank | 5103010 |
| Sequence 12309 | AP000473 | Genbank | 6942329 |
| Sequence 12310 | AP000497 | Genbank | 5926684 |
| Sequence 12311 | AP000525 | Genbank | 5931503 |
| Sequence 12312 | AP000547 | Genbank | 5931525 |
| Sequence 12313 | AP000692 | Genbank | 6693635 |
| Sequence 12314 | AP000962 | Genbank | 6942330 |
| Sequence 12315 | AP001063 | Genbank | 6693613 |
| Sequence 12316 | AP001115 | Genbank | 6899854 |
| Sequence 12317 | AP001136 | Genbank | 6970360 |
| Sequence 12318 | AP001137 | Genbank | 6970361 |
| Sequence 12319 | AP001138 | Genbank | 7262569 |
| Sequence 12320 | AP001172 | Genbank | 6983884 |
| Sequence 12321 | AP001343 | Genbank | 7209831 |
| Sequence 12322 | D00596 | Genbank | 220135 |
| Sequence 12323 | D13119 | Genbank | 285909 |
| Sequence 12324 | D13318 | Genbank | 286026 |
| Sequence 12325 | D13634 | Genbank | 285992 |
| Sequence 12326 | D14658 | Genbank | 285940 |
| Sequence 12327 | D14665 | Genbank | 6630617 |
| Sequence 12328 | D14694 | Genbank | 603801 |
| Sequence 12329 | D14696 | Genbank | 285962 |
| Sequence 12330 | D14710 | Genbank | 559324 |
| Sequence 12331 | D16111 | Genbank | 435637 |
| Sequence 12332 | D16947 | Genbank | 598932 |
| Sequence 12333 | D17652 | Genbank | 409069 |
| Sequence 12334 | D23662 | Genbank | 432362 |
| Sequence 12335 | D25274 | Genbank | 464185 |
| Sequence 12336 | D26600 | Genbank | 565650 |
| Sequence 12337 | D29805 | Genbank | 474986 |
| Sequence 12338 | D31885 | Genbank | 505097 |
| Sequence 12339 | D38112 | Genbank | 644480 |
| Sequence 12340 | D38550 | Genbank | 559708 |
| Sequence 12341 | D42052 | Genbank | 1136740 |
| Sequence 12342 | D42063 | Genbank | 924266 |
| Sequence 12343 | D45131 | Genbank | 1304103 |
| Sequence 12344 | D50916 | Genbank | 1469174 |
| Sequence 12345 | D50929 | Genbank | 1469200 |
| Sequence 12346 | D55673 | Genbank | 870746 |
| Sequence 12347 | D63486 | Genbank | 1469885 |
| Sequence 12348 | D78130 | Genbank | 2443315 |
| Sequence 12349 | D79205 | Genbank | 1754620 |
| Sequence 12350 | D79991 | Genbank | 1136397 |
| Sequence 12351 | D80000 | Genbank | 1136415 |
| Sequence 12352 | D83077 | Genbank | 1304131 |
| Sequence 12353 | D83647 | Genbank | 1209009 |
| Sequence 12354 | D83767 | Genbank | 1913784 |
| Sequence 12355 | D84273 | Genbank | 3894217 |
| Sequence 12356 | D84344 | Genbank | 2340829 |
| Sequence 12357 | D86971 | Genbank | 1504015 |
| Sequence 12358 | D86982 | Genbank | 1504037 |
| Sequence 12359 | D87450 | Genbank | 1665788 |
| Sequence 12360 | D87666 | Genbank | 1620016 |
| Sequence 12361 | D87684 | Genbank | 1663703 |
| Sequence 12362 | D87742 | Genbank | 1665824 |
| Sequence 12363 | E01932 | Genbank | 2170180 |
| Sequence 12364 | E02339 | Genbank | 2170574 |
| Sequence 12365 | E02628 | Genbank | 2170856 |
| Sequence 12366 | E03413 | Genbank | 2171629 |
| Sequence 12367 | E08204 | Genbank | 2176325 |
| Sequence 12368 | E12274 | Genbank | 3251108 |
| Sequence 12369 | E13298 | Genbank | 3252103 |
| Sequence 12370 | E13330 | Genbank | 3252135 |
| Sequence 12371 | E15239 | Genbank | 5709922 |
| Sequence 12372 | J00139 | Genbank | 182715 |
| Sequence 12373 | J01415 | Genbank | 1944628 |
| Sequence 12374 | J02960 | Genbank | 178203 |
| Sequence 12375 | J03537 | Genbank | 337513 |
| Sequence 12376 | J03592 | Genbank | 339722 |
| Sequence 12377 | J03746 | Genbank | 183655 |
| Sequence 12378 | J03779 | Genbank | 179833 |
| Sequence 12379 | J04205 | Genbank | 178686 |
| Sequence 12380 | K00558 | Genbank | 340020 |
| Sequence 12381 | L01057 | Genbank | 292462 |
| Sequence 12382 | L05091 | Genbank | 388030 |
| Sequence 12383 | L05092 | Genbank | 388031 |
| Sequence 12384 | L05628 | Genbank | 1835658 |
| Sequence 12385 | L08125 | Genbank | 306500 |
| Sequence 12386 | L11910 | Genbank | 292420 |
| Sequence 12387 | L12350 | Genbank | 307505 |
| Sequence 12388 | L13806 | Genbank | 306554 |
| Sequence 12389 | L13850 | Genbank | 292165 |
| Sequence 12390 | L14076 | Genbank | 307437 |
| Sequence 12391 | L19185 | Genbank | 440307 |
| Sequence 12392 | L19597 | Genbank | 306467 |
| Sequence 12393 | L20046 | Genbank | 306741 |
| Sequence 12394 | L20941 | Genbank | 507251 |
| Sequence 12395 | L22569 | Genbank | 348706 |
| Sequence 12396 | L25616 | Genbank | 409465 |
| Sequence 12397 | L28809 | Genbank | 454151 |
| Sequence 12398 | L31610 | Genbank | 1220360 |
| Sequence 12399 | L35681 | Genbank | 532031 |
| Sequence 12400 | L38951 | Genbank | 893287 |
| Sequence 12401 | L38995 | Genbank | 704415 |
| Sequence 12402 | L39332 | Genbank | 1235613 |
| Sequence 12403 | L39350 | Genbank | 1237109 |
| Sequence 12404 | L41349 | Genbank | 762825 |
| Sequence 12405 | L43964 | Genbank | 951202 |
| Sequence 12406 | L47647 | Genbank | 1000861 |
| Sequence 12407 | L76416 | Genbank | 5566605 |
| Sequence 12408 | L76568 | Genbank | 1905925 |
| Sequence 12409 | M11058S | Genbank | 184243 |
| Sequence 12410 | M11167 | Genbank | 337381 |
| Sequence 12411 | M11353 | Genbank | 184092 |
| Sequence 12412 | M13519 | Genbank | 179461 |
| Sequence 12413 | M16342 | Genbank | 184266 |
| Sequence 12414 | M16541 | Genbank | 1280204 |
| Sequence 12415 | M17885 | Genbank | 190231 |
| Sequence 12416 | M17886 | Genbank | 190233 |
| Sequence 12417 | M18930 | Genbank | 184371 |
| Sequence 12418 | M19961 | Genbank | 180940 |
| Sequence 12419 | M20496 | Genbank | 809235 |
| Sequence 12420 | M21142 | Genbank | 183402 |
| Sequence 12421 | M22348 | Genbank | 190815 |
| Sequence 12422 | M22760 | Genbank | 695359 |
| Sequence 12423 | M22919 | Genbank | 189016 |
| Sequence 12424 | M24194 | Genbank | 187701 |
| Sequence 12425 | M24486 | Genbank | 190785 |
| Sequence 12426 | M24543 | Genbank | 341200 |
| Sequence 12427 | M25160 | Genbank | 189059 |
| Sequence 12428 | M26481 | Genbank | 619789 |
| Sequence 12429 | M26663 | Genbank | 618463 |
| Sequence 12430 | M27878 | Genbank | 1020144 |
| Sequence 12431 | M30448 | Genbank | 181154 |
| Sequence 12432 | M31520 | Genbank | 337504 |
| Sequence 12433 | M37583 | Genbank | 184059 |
| Sequence 12434 | M38188 | Genbank | 189378 |
| Sequence 12435 | M58485 | Genbank | 180154 |
| Sequence 12436 | M59907 | Genbank | 189383 |
| Sequence 12437 | M60457 | Genbank | 181249 |
| Sequence 12438 | M63005 | Genbank | 179981 |
| Sequence 12439 | M65209 | Genbank | 184444 |
| Sequence 12440 | M74089 | Genbank | 182400 |
| Sequence 12441 | M74775 | Genbank | 187151 |
| Sequence 12442 | M75883 | Genbank | 432974 |
| Sequence 12443 | M77142 | Genbank | 339700 |
| Sequence 12444 | M77836 | Genbank | 189497 |
| Sequence 12445 | M81601 | Genbank | 339442 |
| Sequence 12446 | M90054 | Genbank | 337579 |
| Sequence 12447 | M93036 | Genbank | 182904 |
| Sequence 12448 | M96982 | Genbank | 338262 |
| Sequence 12449 | S62140 | Genbank | 386156 |
| Sequence 12450 | S63912 | Genbank | 399757 |
| Sequence 12451 | S82300 | Genbank | 245388 |
| Sequence 12452 | U02031 | Genbank | 451329 |
| Sequence 12453 | U07151 | Genbank | 460624 |
| Sequence 12454 | U09813 | Genbank | 1008454 |
| Sequence 12455 | U09954 | Genbank | 607792 |
| Sequence 12456 | U12128 | Genbank | 557287 |
| Sequence 12457 | U13616 | Genbank | 608024 |
| Sequence 12458 | U14193 | Genbank | 555899 |
| Sequence 12459 | U14972 | Genbank | 550024 |
| Sequence 12460 | U16798 | Genbank | 806751 |
| Sequence 12461 | U17899 | Genbank | 717053 |
| Sequence 12462 | U19145 | Genbank | 914904 |
| Sequence 12463 | U21914 | Genbank | 736410 |
| Sequence 12464 | U24223 | Genbank | 1215670 |
| Sequence 12465 | U24231 | Genbank | 809486 |
| Sequence 12466 | U25165 | Genbank | 887792 |
| Sequence 12467 | U25182 | Genbank | 799380 |
| Sequence 12468 | U25372 | Genbank | 1045393 |
| Sequence 12469 | U31657 | Genbank | 951331 |
| Sequence 12470 | U33886 | Genbank | 1710107 |
| Sequence 12471 | U34683 | Genbank | 1236349 |
| Sequence 12472 | U35246 | Genbank | 1477465 |
| Sequence 12473 | U37143 | Genbank | 1185451 |
| Sequence 12474 | U37230 | Genbank | 1574941 |
| Sequence 12475 | U38784 | Genbank | 1574947 |
| Sequence 12476 | U41371 | Genbank | 1173904 |
| Sequence 12477 | U52111 | Genbank | 8331754 |
| Sequence 12478 | U52427 | Genbank | 1924973 |
| Sequence 12479 | U54562 | Genbank | 2351381 |
| Sequence 12480 | U54993 | Genbank | 4097314 |
| Sequence 12481 | U55184 | Genbank | 4097327 |
| Sequence 12482 | U55853 | Genbank | 2145094 |
| Sequence 12483 | U57847 | Genbank | 1373420 |
| Sequence 12484 | U61167 | Genbank | 1438934 |
| Sequence 12485 | U65896 | Genbank | 1763689 |
| Sequence 12486 | U66469 | Genbank | 1724072 |
| Sequence 12487 | U66871 | Genbank | 1519518 |
| Sequence 12488 | U68105 | Genbank | 1562509 |
| Sequence 12489 | U68758 | Genbank | 4097815 |
| Sequence 12490 | U69570 | Genbank | 1825473 |
| Sequence 12491 | U70063 | Genbank | 1743866 |
| Sequence 12492 | U73379 | Genbank | 2062372 |
| Sequence 12493 | U73824 | Genbank | 1857236 |
| Sequence 12494 | U80017 | Genbank | 1737211 |
| Sequence 12495 | U80763 | Genbank | 2231366 |
| Sequence 12496 | U81375 | Genbank | 1845344 |
| Sequence 12497 | U82695 | Genbank | 6960456 |
| Sequence 12498 | U90909 | Genbank | 1913889 |
| Sequence 12499 | U91322 | Genbank | 2344876 |
| Sequence 12500 | U91328 | Genbank | 2088550 |
| Sequence 12501 | U94586 | Genbank | 1946691 |
| Sequence 12502 | U95689 | Genbank | 2804669 |
| Sequence 12503 | X00351 | Genbank | 28251 |
| Sequence 12504 | X02152 | Genbank | 34312 |
| Sequence 12505 | X03083 | Genbank | 34309 |
| Sequence 12506 | X06409 | Genbank | 30204 |
| Sequence 12507 | X15822 | Genbank | 30146 |
| Sequence 12508 | X16064 | Genbank | 37495 |
| Sequence 12509 | X52839 | Genbank | 36125 |
| Sequence 12510 | X54540 | Genbank | 37457 |
| Sequence 12511 | X60221 | Genbank | 509290 |
| Sequence 12512 | X61123 | Genbank | 29508 |
| Sequence 12513 | X64707 | Genbank | 29382 |
| Sequence 12514 | X65923 | Genbank | 31302 |
| Sequence 12515 | X66401 | Genbank | 34634 |
| Sequence 12516 | X66503 | Genbank | 415848 |
| Sequence 12517 | X67247 | Genbank | 36149 |
| Sequence 12518 | X68271 | Genbank | 602341 |
| Sequence 12519 | X73114 | Genbank | 402618 |
| Sequence 12520 | X74979 | Genbank | 400462 |
| Sequence 12521 | X75682 | Genbank | 429094 |
| Sequence 12522 | X75861 | Genbank | 456258 |
| Sequence 12523 | X76488 | Genbank | 434305 |
| Sequence 12524 | X80199 | Genbank | 2385366 |
| Sequence 12525 | X81900 | Genbank | 2274973 |
| Sequence 12526 | X84908 | Genbank | 1502344 |
| Sequence 12527 | X98356 | Genbank | 1648869 |
| Sequence 12528 | X99585 | Genbank | 1770518 |
| Sequence 12529 | Y00815 | Genbank | 34266 |
| Sequence 12530 | Y15286 | Genbank | 2584788 |
| Sequence 12531 | Y17448 | Genbank | 5002578 |
| Sequence 12532 | Z13009 | Genbank | 31072 |
| Sequence 12533 | Z14244 | Genbank | 30150 |
| Sequence 12534 | Z48570 | Genbank | 695580 |
| Sequence 12535 | Z56866 | Genbank | 1028097 |
| Sequence 12536 | Z58044 | Genbank | 1029275 |
| Sequence 12537 | Z68277 | Genbank | 1130689 |
| Sequence 12538 | Z69709 | Genbank | 1204107 |
| Sequence 12539 | Z70233 | Genbank | 1235542 |
| Sequence 12540 | Z73417 | Genbank | 1322397 |
| Sequence 12541 | Z82214 | Genbank | 5911819 |
| Sequence 12542 | Z83313 | Genbank | 1730462 |
| Sequence 12543 | Z83843 | Genbank | 2578095 |
| Sequence 12544 | Z83848 | Genbank | 3164072 |
| Sequence 12545 | Z83851 | Genbank | 5441348 |
| Sequence 12546 | Z84466 | Genbank | 3319673 |
| Sequence 12547 | Z84812 | Genbank | 1845164 |
| Sequence 12548 | Z85999 | Genbank | 2326514 |
| Sequence 12549 | Z92544 | Genbank | 1869775 |
| Sequence 12550 | Z95126 | Genbank | 2342581 |
| Sequence 12551 | Z97053 | Genbank | 9650676 |
| Sequence 12552 | Z98749 | Genbank | 4775611 |
| Sequence 12553 | Z98950 | Genbank | 3036783 |
| Sequence 12554 | Z99571 | Genbank | 2791262 |
| Sequence 12555 | AA001709 | EST | 1445523 |
| Sequence 12556 | AA002222 | EST | 1445157 |
| Sequence 12557 | AA004427 | EST | 1448541 |
| Sequence 12558 | AA005278 | EST | 1448740 |
| Sequence 12559 | AA007226 | EST | 1463200 |
| Sequence 12560 | AA010519 | EST | 1471545 |
| Sequence 12561 | AA011539 | EST | 1472565 |
| Sequence 12562 | AA018969 | EST | 1482361 |
| Sequence 12563 | AA020756 | EST | 1485332 |
| Sequence 12564 | AA022472 | EST | 1486571 |
| Sequence 12565 | AA024695 | EST | 1489608 |
| Sequence 12566 | AA025361 | EST | 1489375 |
| Sequence 12567 | AA025400 | EST | 1491566 |
| Sequence 12568 | AA025432 | EST | 1490914 |
| Sequence 12569 | AA026215 | EST | 1492901 |
| Sequence 12570 | AA026422 | EST | 1492323 |
| Sequence 12571 | AA027229 | EST | 1492149 |
| Sequence 12572 | AA028138 | EST | 1494207 |
| Sequence 12573 | AA029944 | EST | 1496172 |
| Sequence 12574 | AA035377 | EST | 1507101 |
| Sequence 12575 | AA036809 | EST | 1510048 |
| Sequence 12576 | AA039705 | EST | 1515984 |
| Sequence 12577 | AA039716 | EST | 1515995 |
| Sequence 12578 | AA040270 | EST | 1516675 |
| Sequence 12579 | AA040302 | EST | 1516747 |
| Sequence 12580 | AA040863 | EST | 1517159 |
| Sequence 12581 | AA044237 | EST | 1522113 |
| Sequence 12582 | AA044842 | EST | 1523064 |
| Sequence 12583 | AA045223 | EST | 1523629 |
| Sequence 12584 | AA045780 | EST | 1525739 |
| Sequence 12585 | AA046883 | EST | 1524818 |
| Sequence 12586 | AA046936 | EST | 1524835 |
| Sequence 12587 | AA047046 | EST | 1524944 |
| Sequence 12588 | AA047224 | EST | 1525124 |
| Sequence 12589 | AA047729 | EST | 1527526 |
| Sequence 12590 | AA053607 | EST | 1544534 |
| Sequence 12591 | AA054470 | EST | 1545395 |
| Sequence 12592 | AA055504 | EST | 1547843 |
| Sequence 12593 | AA055943 | EST | 1548282 |
| Sequence 12594 | AA057400 | EST | 1550105 |
| Sequence 12595 | AA059199 | EST | 1552077 |
| Sequence 12598 | AA069597 | EST | 1576956 |
| Sequence 12597 | AA074189 | EST | 1614057 |
| Sequence 12598 | AA074611 | EST | 1614677 |
| Sequence 12599 | AA074614 | EST | 1614492 |
| Sequence 12600 | AA075494 | EST | 1615364 |
| Sequence 12601 | AA079690 | EST | 1618711 |
| Sequence 12602 | AA081037 | EST | 1622972 |
| Sequence 12603 | AA082177 | EST | 1624236 |
| Sequence 12604 | AA082640 | EST | 1624905 |
| Sequence 12605 | AA083420 | EST | 1625481 |
| Sequence 12606 | AA084868 | EST | 1626988 |
| Sequence 12607 | AA085763 | EST | 1629225 |
| Sequence 12608 | AA085810 | EST | 1629342 |
| Sequence 12609 | AA088187 | EST | 1633745 |
| Sequence 12610 | AA088758 | EST | 1634279 |
| Sequence 12611 | AA088843 | EST | 1634355 |
| Sequence 12612 | AA091442 | EST | 1636897 |
| Sequence 12613 | AA092589 | EST | 1637410 |
| Sequence 12614 | AA093037 | EST | 1638242 |
| Sequence 12615 | AA093542 | EST | 1639127 |
| Sequence 12616 | AA094831 | EST | 1640416 |
| Sequence 12617 | AA094888 | EST | 1640465 |
| Sequence 12618 | AA095725 | EST | 1641310 |
| Sequence 12619 | AA113354 | EST | 1665194 |
| Sequence 12620 | AA115636 | EST | 1670771 |
| Sequence 12621 | AA120954 | EST | 1678359 |
| Sequence 12622 | AA121266 | EST | 1678899 |
| Sequence 12623 | AA126086 | EST | 1685760 |
| Sequence 12624 | AA127483 | EST | 1686755 |
| Sequence 12625 | AA127687 | EST | 1687021 |
| Sequence 12626 | AA127718 | EST | 1687007 |
| Sequence 12627 | AA128745 | EST | 1688653 |
| Sequence 12628 | AA132163 | EST | 1693672 |
| Sequence 12629 | AA133361 | EST | 1690329 |
| Sequence 12630 | AA134275 | EST | 1691754 |
| Sequence 12631 | AA135477 | EST | 1696553 |
| Sequence 12632 | AA135809 | EST | 1696820 |
| Sequence 12633 | AA136084 | EST | 1697294 |
| Sequence 12634 | AA136337 | EST | 1697545 |
| Sequence 12635 | AA147775 | EST | 1717155 |
| Sequence 12636 | AA150361 | EST | 1721873 |
| Sequence 12637 | AA150556 | EST | 1722069 |
| Sequence 12638 | AA151565 | EST | 1720183 |
| Sequence 12639 | AA152225 | EST | 1721426 |
| Sequence 12640 | AA156901 | EST | 1728581 |
| Sequence 12641 | AA166664 | EST | 1745119 |
| Sequence 12642 | AA166955 | EST | 1745331 |
| Sequence 12643 | AA167011 | EST | 1745386 |
| Sequence 12644 | AA167399 | EST | 1745846 |
| Sequence 12645 | AA169486 | EST | 1748200 |
| Sequence 12646 | AA172003 | EST | 1751060 |
| Sequence 12647 | AA173437 | EST | 1753566 |
| Sequence 12648 | AA173705 | EST | 1753883 |
| Sequence 12649 | AA174161 | EST | 1754441 |
| Sequence 12650 | AA177042 | EST | 1758180 |
| Sequence 12651 | AA178986 | EST | 1760538 |
| Sequence 12652 | AA179059 | EST | 1760471 |
| Sequence 12653 | AA179250 | EST | 1760619 |
| Sequence 12654 | AA179888 | EST | 1761212 |
| Sequence 12655 | AA180349 | EST | 1761631 |
| Sequence 12656 | AA181138 | EST | 1764605 |
| Sequence 12657 | AA182751 | EST | 1766581 |
| Sequence 12658 | AA186328 | EST | 1774446 |
| Sequence 12659 | AA187076 | EST | 1775245 |
| Sequence 12660 | AA189093 | EST | 1776127 |
| Sequence 12661 | AA191365 | EST | 1780231 |
| Sequence 12662 | AA191629 | EST | 1780293 |
| Sequence 12663 | AA192253 | EST | 1782078 |
| Sequence 12664 | AA193193 | EST | 1782646 |
| Sequence 12665 | AA195119 | EST | 1784830 |
| Sequence 12666 | AA203650 | EST | 1799369 |
| Sequence 12667 | AA203671 | EST | 1799390 |
| Sequence 12668 | AA206446 | EST | 1801826 |
| Sequence 12669 | AA206761 | EST | 1802148 |
| Sequence 12670 | AA215640 | EST | 1815385 |
| Sequence 12671 | AA216102 | EST | 1816057 |
| Sequence 12672 | AA216292 | EST | 1816271 |
| Sequence 12673 | AA216350 | EST | 1816315 |
| Sequence 12674 | AA223194 | EST | 1843737 |
| Sequence 12675 | AA224460 | EST | 1845078 |
| Sequence 12676 | AA226801 | EST | 1848430 |
| Sequence 12677 | AA232991 | EST | 1856115 |
| Sequence 12678 | AA233294 | EST | 1856287 |
| Sequence 12679 | AA233487 | EST | 1856517 |
| Sequence 12680 | AA233674 | EST | 1856667 |
| Sequence 12681 | AA236252 | EST | 1858329 |
| Sequence 12682 | AA242860 | EST | 1873678 |
| Sequence 12683 | AA243369 | EST | 1874162 |
| Sequence 12684 | AA243646 | EST | 1874456 |
| Sequence 12685 | AA248173 | EST | 1878762 |
| Sequence 12686 | AA251702 | EST | 1886665 |
| Sequence 12687 | AA252745 | EST | 1887715 |
| Sequence 12688 | AA255738 | EST | 1892676 |
| Sequence 12689 | AA256348 | EST | 1891933 |
| Sequence 12690 | AA258029 | EST | 1894479 |
| Sequence 12691 | AA258243 | EST | 1894238 |
| Sequence 12692 | AA280080 | EST | 1921554 |
| Sequence 12693 | AA280848 | EST | 1923546 |
| Sequence 12694 | AA281553 | EST | 1924231 |
| Sequence 12695 | AA283976 | EST | 1928258 |
| Sequence 12696 | AA284242 | EST | 1928542 |
| Sequence 12697 | AA285040 | EST | 1927721 |
| Sequence 12698 | AA285126 | EST | 1928107 |
| Sequence 12699 | AA287500 | EST | 1933225 |
| Sequence 12700 | AA287921 | EST | 1933744 |
| Sequence 12701 | AA287941 | EST | 1933764 |
| Sequence 12702 | AA291316 | EST | 1939294 |
| Sequence 12703 | AA292003 | EST | 1939980 |
| Sequence 12704 | AA292069 | EST | 1940055 |
| Sequence 12705 | AA293612 | EST | 1941263 |
| Sequence 12706 | AA295997 | EST | 1948334 |
| Sequence 12707 | AA298183 | EST | 1950600 |
| Sequence 12708 | AA299920 | EST | 1952324 |
| Sequence 12709 | AA303377 | EST | 1955711 |
| Sequence 12710 | AA307371 | EST | 1959720 |
| Sequence 12711 | AA307611 | EST | 1959960 |
| Sequence 12712 | AA311453 | EST | 1963779 |
| Sequence 12713 | AA312681 | EST | 1965030 |
| Sequence 12714 | AA313693 | EST | 1966022 |
| Sequence 12715 | AA314391 | EST | 1966740 |
| Sequence 12716 | AA316545 | EST | 1968873 |
| Sequence 12717 | AA316727 | EST | 1969286 |
| Sequence 12718 | AA316742 | EST | 1969141 |
| Sequence 12719 | AA316868 | EST | 1969196 |
| Sequence 12720 | AA317660 | EST | 1969986 |
| Sequence 12721 | AA318533 | EST | 1970882 |
| Sequence 12722 | AA318684 | EST | 1971012 |
| Sequence 12723 | AA319233 | EST | 1971558 |
| Sequence 12724 | AA319855 | EST | 1972181 |
| Sequence 12725 | AA327986 | EST | 1980229 |
| Sequence 12726 | AA328203 | EST | 1980603 |
| Sequence 12727 | AA345360 | EST | 1997595 |
| Sequence 12728 | AA346874 | EST | 1999111 |
| Sequence 12729 | AA347532 | EST | 1999862 |
| Sequence 12730 | AA364154 | EST | 2016503 |
| Sequence 12731 | AA364506 | EST | 2016845 |
| Sequence 12732 | AA370825 | EST | 2023163 |
| Sequence 12733 | AA373288 | EST | 2025893 |
| Sequence 12734 | AA380652 | EST | 2032972 |
| Sequence 12735 | AA383018 | EST | 2035335 |
| Sequence 12736 | AA393236 | EST | 2046205 |
| Sequence 12737 | AA393310 | EST | 2046278 |
| Sequence 12738 | AA393525 | EST | 2046493 |
| Sequence 12739 | AA393906 | EST | 2047017 |
| Sequence 12740 | AA398315 | EST | 2051620 |
| Sequence 12741 | AA398413 | EST | 2051522 |
| Sequence 12742 | AA398612 | EST | 2051721 |
| Sequence 12743 | AA400644 | EST | 2054515 |
| Sequence 12744 | AA401361 | EST | 2053569 |
| Sequence 12745 | AA402539 | EST | 2056345 |
| Sequence 12746 | AA402743 | EST | 2057061 |
| Sequence 12747 | AA402800 | EST | 2056553 |
| Sequence 12748 | AA405045 | EST | 2063467 |
| Sequence 12749 | AA405814 | EST | 2063797 |
| Sequence 12750 | AA406310 | EST | 2064294 |
| Sequence 12751 | AA410936 | EST | 2070190 |
| Sequence 12752 | AA417743 | EST | 2079544 |
| Sequence 12753 | AA418533 | EST | 2080314 |
| Sequence 12754 | AA421297 | EST | 2100139 |
| Sequence 12755 | AA427471 | EST | 2111333 |
| Sequence 12756 | AA427574 | EST | 2112002 |
| Sequence 12757 | AA428516 | EST | 2112513 |
| Sequence 12758 | AA430552 | EST | 2111127 |
| Sequence 12759 | AA432282 | EST | 2114670 |
| Sequence 12760 | AA434140 | EST | 2139054 |
| Sequence 12761 | AA434447 | EST | 2139361 |
| Sequence 12762 | AA435671 | EST | 2140585 |
| Sequence 12763 | AA442377 | EST | 2154255 |
| Sequence 12764 | AA442786 | EST | 2155461 |
| Sequence 12765 | AA442878 | EST | 2155553 |
| Sequence 12766 | AA447199 | EST | 2159864 |
| Sequence 12767 | AA448292 | EST | 2161962 |
| Sequence 12768 | AA448409 | EST | 2162079 |
| Sequence 12769 | AA453183 | EST | 2166852 |
| Sequence 12770 | AA453411 | EST | 2167080 |
| Sequence 12771 | AA453747 | EST | 2167416 |
| Sequence 12772 | AA454187 | EST | 2167856 |
| Sequence 12773 | AA456524 | EST | 2179100 |
| Sequence 12774 | AA458612 | EST | 2183519 |
| Sequence 12775 | AA459528 | EST | 2184435 |
| Sequence 12776 | AA460187 | EST | 2185003 |
| Sequence 12777 | AA460266 | EST | 2185082 |
| Sequence 12778 | AA463256 | EST | 2188140 |
| Sequence 12779 | AA463899 | EST | 2188783 |
| Sequence 12780 | AA465606 | EST | 2191773 |
| Sequence 12781 | AA467764 | EST | 2194298 |
| Sequence 12782 | AA468230 | EST | 2194764 |
| Sequence 12783 | AA468757 | EST | 2195291 |
| Sequence 12784 | AA470337 | EST | 2197646 |
| Sequence 12785 | AA477608 | EST | 2206242 |
| Sequence 12786 | AA478103 | EST | 2206737 |
| Sequence 12787 | AA478663 | EST | 2207297 |
| Sequence 12788 | AA478669 | EST | 2207303 |
| Sequence 12789 | AA480081 | EST | 2208232 |
| Sequence 12790 | AA483251 | EST | 2212064 |
| Sequence 12791 | AA483698 | EST | 2212511 |
| Sequence 12792 | AA486416 | EST | 2216580 |
| Sequence 12793 | AA491100 | EST | 2220273 |
| Sequence 12794 | AA491505 | EST | 2218288 |
| Sequence 12795 | AA493382 | EST | 2223223 |
| Sequence 12796 | AA493522 | EST | 2223363 |
| Sequence 12797 | AA494118 | EST | 2223959 |
| Sequence 12798 | AA494433 | EST | 2224220 |
| Sequence 12799 | AA496993 | EST | 2230314 |
| Sequence 12800 | AA501429 | EST | 2236396 |
| Sequence 12801 | AA503827 | EST | 2238794 |
| Sequence 12802 | AA505115 | EST | 2241275 |
| Sequence 12803 | AA507605 | EST | 2244044 |
| Sequence 12804 | AA507791 | EST | 2244230 |
| Sequence 12805 | AA513367 | EST | 2251779 |
| Sequence 12806 | AA513447 | EST | 2251859 |
| Sequence 12807 | AA514215 | EST | 2253883 |
| Sequence 12808 | AA514331 | EST | 2253839 |
| Sequence 12809 | AA514555 | EST | 2254155 |
| Sequence 12810 | AA514835 | EST | 2254435 |
| Sequence 12811 | AA514972 | EST | 2254572 |
| Sequence 12812 | AA515971 | EST | 2255571 |
| Sequence 12813 | AA521142 | EST | 2261685 |
| Sequence 12814 | AA522471 | EST | 2263183 |
| Sequence 12815 | AA523897 | EST | 2264825 |
| Sequence 12816 | AA524190 | EST | 2265118 |
| Sequence 12817 | AA525960 | EST | 2268029 |
| Sequence 12818 | AA526064 | EST | 2268133 |
| Sequence 12819 | AA526186 | EST | 2268255 |
| Sequence 12820 | AA526891 | EST | 2268960 |
| Sequence 12821 | AA526943 | EST | 2269012 |
| Sequence 12822 | AA527040 | EST | 2269109 |
| Sequence 12823 | AA528775 | EST | 2270844 |
| Sequence 12824 | AA531540 | EST | 2274246 |
| Sequence 12825 | AA533286 | EST | 2277382 |
| Sequence 12826 | AA533355 | EST | 2277451 |
| Sequence 12827 | AA534137 | EST | 2278153 |
| Sequence 12828 | AA535571 | EST | 2279824 |
| Sequence 12829 | AA541666 | EST | 2288100 |
| Sequence 12830 | AA541813 | EST | 2288247 |
| Sequence 12831 | AA548722 | EST | 2319004 |
| Sequence 12832 | AA551733 | EST | 2321985 |
| Sequence 12833 | AA552169 | EST | 2322421 |
| Sequence 12834 | AA554726 | EST | 2325265 |
| Sequence 12835 | AA555194 | EST | 2325733 |
| Sequence 12836 | AA557191 | EST | 2327668 |
| Sequence 12837 | AA563905 | EST | 2335544 |
| Sequence 12838 | AA564118 | EST | 2335757 |
| Sequence 12839 | AA565885 | EST | 2337524 |
| Sequence 12840 | AA568360 | EST | 2341414 |
| Sequence 12841 | AA568578 | EST | 2341632 |
| Sequence 12842 | AA570171 | EST | 2344151 |
| Sequence 12843 | AA577170 | EST | 2354644 |
| Sequence 12844 | AA577927 | EST | 2356111 |
| Sequence 12845 | AA579876 | EST | 2355203 |
| Sequence 12846 | AA580408 | EST | 2355735 |
| Sequence 12847 | AA582370 | EST | 2359730 |
| Sequence 12848 | AA583169 | EST | 2367778 |
| Sequence 12849 | AA586921 | EST | 2397735 |
| Sequence 12850 | AA587325 | EST | 2398139 |
| Sequence 12851 | AA588161 | EST | 2402336 |
| Sequence 12852 | AA588541 | EST | 2401716 |
| Sequence 12853 | AA595339 | EST | 2410689 |
| Sequence 12854 | AA599217 | EST | 2432842 |
| Sequence 12855 | AA602418 | EST | 2435914 |
| Sequence 12856 | AA604553 | EST | 2445417 |
| Sequence 12857 | AA608603 | EST | 2457031 |
| Sequence 12858 | AA608917 | EST | 2457345 |
| Sequence 12859 | AA608990 | EST | 2457418 |
| Sequence 12860 | AA610801 | EST | 2459229 |
| Sequence 12861 | AA613587 | EST | 2464625 |
| Sequence 12862 | AA613637 | EST | 2464675 |
| Sequence 12863 | AA614729 | EST | 2466925 |
| Sequence 12864 | AA626535 | EST | 2538922 |
| Sequence 12865 | AA627229 | EST | 2540273 |
| Sequence 12866 | AA627804 | EST | 2539899 |
| Sequence 12867 | AA627996 | EST | 2539995 |
| Sequence 12868 | AA631218 | EST | 2553829 |
| Sequence 12869 | AA631224 | EST | 2553835 |
| Sequence 12870 | AA631404 | EST | 2554015 |
| Sequence 12871 | AA631684 | EST | 2554295 |
| Sequence 12872 | AA632005 | EST | 2555419 |
| Sequence 12873 | AA632225 | EST | 2555639 |
| Sequence 12874 | AA632328 | EST | 2555742 |
| Sequence 12875 | AA633317 | EST | 2556731 |
| Sequence 12876 | AA633888 | EST | 2557102 |
| Sequence 12877 | AA634120 | EST | 2557334 |
| Sequence 12878 | AA634360 | EST | 2557574 |
| Sequence 12879 | AA635630 | EST | 2559472 |
| Sequence 12880 | AA636029 | EST | 2559871 |
| Sequence 12881 | AA639730 | EST | 2563509 |
| Sequence 12882 | AA640721 | EST | 2565971 |
| Sequence 12883 | AA640779 | EST | 2566029 |
| Sequence 12884 | AA642858 | EST | 2568076 |
| Sequence 12885 | AA643157 | EST | 2568375 |
| Sequence 12886 | AA643611 | EST | 2568829 |
| Sequence 12887 | AA643763 | EST | 2568981 |
| Sequence 12888 | AA649327 | EST | 2575756 |
| Sequence 12889 | AA653201 | EST | 2589372 |
| Sequence 12890 | AA655033 | EST | 2591187 |
| Sequence 12891 | AA657408 | EST | 2593562 |
| Sequence 12892 | AA659388 | EST | 2595542 |
| Sequence 12893 | AA662764 | EST | 2616755 |
| Sequence 12894 | AA669846 | EST | 2631345 |
| Sequence 12895 | AA669862 | EST | 2631361 |
| Sequence 12896 | AA678121 | EST | 2658643 |
| Sequence 12897 | AA678246 | EST | 2658768 |
| Sequence 12898 | AA678311 | EST | 2658833 |
| Sequence 12899 | AA678696 | EST | 2659218 |
| Sequence 12900 | AA682980 | EST | 2668871 |
| Sequence 12901 | AA683334 | EST | 2669225 |
| Sequence 12902 | AA687631 | EST | 2674537 |
| Sequence 12903 | AA688365 | EST | 2675271 |
| Sequence 12904 | AA694202 | EST | 2695140 |
| Sequence 12905 | AA699532 | EST | 2702655 |
| Sequence 12906 | AA699712 | EST | 2702675 |
| Sequence 12907 | AA700166 | EST | 2703129 |
| Sequence 12908 | AA701936 | EST | 2705049 |
| Sequence 12909 | AA702410 | EST | 2705523 |
| Sequence 12910 | AA702881 | EST | 2705994 |
| Sequence 12911 | AA704096 | EST | 2714014 |
| Sequence 12912 | AA705746 | EST | 2715664 |
| Sequence 12913 | AA707188 | EST | 2717106 |
| Sequence 12914 | AA708421 | EST | 2718339 |
| Sequence 12915 | AA714951 | EST | 2727225 |
| Sequence 12916 | AA723194 | EST | 2740971 |
| Sequence 12917 | AA731317 | EST | 2752521 |
| Sequence 12918 | AA731688 | EST | 2752577 |
| Sequence 12919 | AA736880 | EST | 2768114 |
| Sequence 12920 | AA748046 | EST | 2788004 |
| Sequence 12921 | AA749025 | EST | 2788983 |
| Sequence 12922 | AA764734 | EST | 2815972 |
| Sequence 12923 | AA769434 | EST | 2820672 |
| Sequence 12924 | AA770613 | EST | 2821851 |
| Sequence 12925 | AA772280 | EST | 2824063 |
| Sequence 12926 | AA773040 | EST | 2824611 |
| Sequence 12927 | AA775624 | EST | 2834958 |
| Sequence 12928 | AA775824 | EST | 2835158 |
| Sequence 12929 | AA777499 | EST | 2836978 |
| Sequence 12930 | AA778560 | EST | 2837891 |
| Sequence 12931 | AA778776 | EST | 2838107 |
| Sequence 12932 | AA788754 | EST | 2848874 |
| Sequence 12933 | AA806729 | EST | 2875479 |
| Sequence 12934 | AA806878 | EST | 2876454 |
| Sequence 12935 | AA808206 | EST | 2877612 |
| Sequence 12936 | AA809267 | EST | 2878673 |
| Sequence 12937 | AA809700 | EST | 2879106 |
| Sequence 12938 | AA809964 | EST | 2879370 |
| Sequence 12939 | AA810073 | EST | 2879479 |
| Sequence 12940 | AA811224 | EST | 2880835 |
| Sequence 12941 | AA811879 | EST | 2881490 |
| Sequence 12942 | AA813197 | EST | 2883182 |
| Sequence 12943 | AA814246 | EST | 2883842 |
| Sequence 12944 | AA814643 | EST | 2884239 |
| Sequence 12945 | AA825680 | EST | 2898992 |
| Sequence 12946 | AA828003 | EST | 2900366 |
| Sequence 12947 | AA828330 | EST | 2901429 |
| Sequence 12948 | AA829737 | EST | 2902836 |
| Sequence 12949 | AA831926 | EST | 2905025 |
| Sequence 12950 | AA831948 | EST | 2905047 |
| Sequence 12951 | AA832521 | EST | 2898667 |
| Sequence 12952 | AA836590 | EST | 2910909 |
| Sequence 12953 | AA837128 | EST | 2912327 |
| Sequence 12954 | AA843115 | EST | 2929633 |
| Sequence 12955 | AA843807 | EST | 2930258 |
| Sequence 12956 | AA846016 | EST | 2932156 |
| Sequence 12957 | AA846735 | EST | 2932875 |
| Sequence 12958 | AA854511 | EST | 2942049 |
| Sequence 12959 | AA861279 | EST | 2953419 |
| Sequence 12960 | AA863230 | EST | 2955709 |
| Sequence 12961 | AA863377 | EST | 2955856 |
| Sequence 12962 | AA865569 | EST | 2957845 |
| Sequence 12963 | AA872000 | EST | 2968038 |
| Sequence 12964 | AA873173 | EST | 2969295 |
| Sequence 12965 | AA873489 | EST | 2969611 |
| Sequence 12966 | AA876033 | EST | 2984796 |
| Sequence 12967 | AA877009 | EST | 2986086 |
| Sequence 12968 | AA877239 | EST | 2986316 |
| Sequence 12969 | AA877769 | EST | 2986734 |
| Sequence 12970 | AA883195 | EST | 2994671 |
| Sequence 12971 | AA884623 | EST | 2994153 |
| Sequence 12972 | AA885297 | EST | 2994374 |
| Sequence 12973 | AA886074 | EST | 3001182 |
| Sequence 12974 | AA890342 | EST | 3017221 |
| Sequence 12975 | AA894745 | EST | 3031146 |
| Sequence 12976 | AA902974 | EST | 3038097 |
| Sequence 12977 | AA903301 | EST | 3038424 |
| Sequence 12978 | AA910691 | EST | 3049981 |
| Sequence 12979 | AA910881 | EST | 3050171 |
| Sequence 12980 | AA917382 | EST | 3057272 |
| Sequence 12981 | AA931246 | EST | 3085632 |
| Sequence 12982 | AA931567 | EST | 3085953 |
| Sequence 12983 | AA933730 | EST | 3089998 |
| Sequence 12984 | AA935696 | EST | 3092853 |
| Sequence 12985 | AA937407 | EST | 3095518 |
| Sequence 12986 | AA946596 | EST | 3109991 |
| Sequence 12987 | AA947190 | EST | 3110585 |
| Sequence 12988 | AA948676 | EST | 3109929 |
| Sequence 12989 | AA954463 | EST | 3118158 |
| Sequence 12990 | AA969495 | EST | 3144675 |
| Sequence 12991 | AA969966 | EST | 3145479 |
| Sequence 12992 | AA972733 | EST | 3147913 |
| Sequence 12993 | AA973170 | EST | 3148350 |
| Sequence 12994 | AA983457 | EST | 3161982 |
| Sequence 12995 | AA987257 | EST | 3172621 |
| Sequence 12996 | AA988988 | EST | 3174559 |
| Sequence 12997 | AA992853 | EST | 3179398 |
| Sequence 12998 | AA992960 | EST | 3179505 |
| Sequence 12999 | AA995518 | EST | 3182007 |
| Sequence 13000 | AI003314 | EST | 3203728 |
| Sequence 13001 | AI005654 | EST | 3215164 |
| Sequence 13002 | AI016909 | EST | 3231245 |
| Sequence 13003 | AI018676 | EST | 3232474 |
| Sequence 13004 | AI025856 | EST | 3241469 |
| Sequence 13005 | AI032664 | EST | 3253790 |
| Sequence 13006 | AI034460 | EST | 3255413 |
| Sequence 13007 | AI039964 | EST | 3279158 |
| Sequence 13008 | AI040839 | EST | 3280033 |
| Sequence 13009 | AI041671 | EST | 3280865 |
| Sequence 13010 | AI041813 | EST | 3281007 |
| Sequence 13011 | AI052135 | EST | 3308126 |
| Sequence 13012 | AI052256 | EST | 3308247 |
| Sequence 13013 | AI052761 | EST | 3308752 |
| Sequence 13014 | AI053744 | EST | 3321531 |
| Sequence 13015 | AI057607 | EST | 3331473 |
| Sequence 13016 | AI061586 | EST | 6358883 |
| Sequence 13017 | AI061642 | EST | 6358939 |
| Sequence 13018 | AI064691 | EST | 6358963 |
| Sequence 13019 | AI064781 | EST | 6359053 |
| Sequence 13020 | AI074143 | EST | 3400787 |
| Sequence 13021 | AI074253 | EST | 3400897 |
| Sequence 13022 | AI074350 | EST | 3400994 |
| Sequence 13023 | AI075208 | EST | 3401799 |
| Sequence 13024 | AI076009 | EST | 3405187 |
| Sequence 13025 | AI076793 | EST | 3405971 |
| Sequence 13026 | AI079885 | EST | 3416136 |
| Sequence 13027 | AI080676 | EST | 3416927 |
| Sequence 13028 | AI081536 | EST | 3418328 |
| Sequence 13029 | AI086009 | EST | 3424432 |
| Sequence 13030 | AI086963 | EST | 3425386 |
| Sequence 13031 | AI087104 | EST | 3425527 |
| Sequence 13032 | AI091297 | EST | 3430356 |
| Sequence 13033 | AI091340 | EST | 3430399 |
| Sequence 13034 | AI096751 | EST | 3445859 |
| Sequence 13035 | AI114651 | EST | 6359996 |
| Sequence 13036 | AI123912 | EST | 3539678 |
| Sequence 13037 | AI126019 | EST | 3594533 |
| Sequence 13038 | AI127959 | EST | 3596473 |
| Sequence 13039 | AI133330 | EST | 6360646 |
| Sequence 13040 | AI139392 | EST | 3645364 |
| Sequence 13041 | AI143894 | EST | 3665703 |
| Sequence 13042 | AI144216 | EST | 3666025 |
| Sequence 13043 | AI146737 | EST | 3674419 |
| Sequence 13044 | AI148343 | EST | 3676025 |
| Sequence 13045 | AI159872 | EST | 3693252 |
| Sequence 13046 | AI160110 | EST | 3693490 |
| Sequence 13047 | AI168423 | EST | 3701593 |
| Sequence 13048 | AI183462 | EST | 3734100 |
| Sequence 13049 | AI184770 | EST | 3735408 |
| Sequence 13050 | AI186840 | EST | 3737478 |
| Sequence 13051 | AI187815 | EST | 3739024 |
| Sequence 13052 | AI189478 | EST | 3740687 |
| Sequence 13053 | AI191342 | EST | 3742551 |
| Sequence 13054 | AI198812 | EST | 3751418 |
| Sequence 13055 | AI198882 | EST | 3751488 |
| Sequence 13056 | AI202429 | EST | 3755035 |
| Sequence 13057 | AI202937 | EST | 3755543 |
| Sequence 13058 | AI207682 | EST | 6361696 |
| Sequence 13059 | AI216387 | EST | 3785428 |
| Sequence 13060 | AI221087 | EST | 3803290 |
| Sequence 13061 | AI240307 | EST | 3835704 |
| Sequence 13062 | AI240817 | EST | 3836214 |
| Sequence 13063 | AI242075 | EST | 3837472 |
| Sequence 13064 | AI244187 | EST | 3839584 |
| Sequence 13065 | AI246248 | EST | 3841645 |
| Sequence 13066 | AI247411 | EST | 3842808 |
| Sequence 13067 | AI248525 | EST | 3843922 |
| Sequence 13068 | AI249008 | EST | 3844405 |
| Sequence 13069 | AI251793 | EST | 3848322 |
| Sequence 13070 | AI253046 | EST | 3849575 |
| Sequence 13071 | AI253330 | EST | 3850451 |
| Sequence 13072 | AI253436 | EST | 3850391 |
| Sequence 13073 | AI262640 | EST | 3870843 |
| Sequence 13074 | AI267506 | EST | 3886673 |
| Sequence 13075 | AI267570 | EST | 3886737 |
| Sequence 13076 | AI268542 | EST | 3887709 |
| Sequence 13077 | AI269205 | EST | 3888372 |
| Sequence 13078 | AI275967 | EST | 3898241 |
| Sequence 13079 | AI275998 | EST | 3898272 |
| Sequence 13080 | AI276110 | EST | 3898384 |
| Sequence 13081 | AI278317 | EST | 3900585 |
| Sequence 13082 | AI278766 | EST | 3917000 |
| Sequence 13083 | AI278998 | EST | 3917232 |
| Sequence 13084 | AI280044 | EST | 3918277 |
| Sequence 13085 | AI280747 | EST | 3918980 |
| Sequence 13086 | AI281464 | EST | 3919697 |
| Sequence 13087 | AI282246 | EST | 3920479 |
| Sequence 13088 | AI282254 | EST | 3920487 |
| Sequence 13089 | AI285371 | EST | 3923604 |
| Sequence 13090 | AI288305 | EST | 3930985 |
| Sequence 13091 | AI289108 | EST | 3932372 |
| Sequence 13092 | AI290733 | EST | 3933507 |
| Sequence 13093 | AI291613 | EST | 3934387 |
| Sequence 13094 | AI291918 | EST | 3934692 |
| Sequence 13095 | AI302808 | EST | 3962154 |
| Sequence 13096 | AI309128 | EST | 4003999 |
| Sequence 13097 | AI335458 | EST | 4072385 |
| Sequence 13098 | AI337195 | EST | 4074122 |
| Sequence 13099 | AI339940 | EST | 4076867 |
| Sequence 13100 | AI341735 | EST | 4078662 |
| Sequence 13101 | AI342244 | EST | 4079171 |
| Sequence 13102 | AI344445 | EST | 4081651 |
| Sequence 13103 | AI345345 | EST | 4082551 |
| Sequence 13104 | AI345677 | EST | 4082883 |
| Sequence 13105 | AI356050 | EST | 4107671 |
| Sequence 13106 | AI361513 | EST | 4113134 |
| Sequence 13107 | AI370743 | EST | 4149496 |
| Sequence 13108 | AI380670 | EST | 4190523 |
| Sequence 13109 | AI381627 | EST | 4194408 |
| Sequence 13110 | AI384017 | EST | 4196798 |
| Sequence 13111 | AI400359 | EST | 4243446 |
| Sequence 13112 | AI420311 | EST | 4266242 |
| Sequence 13113 | AI421401 | EST | 4267332 |
| Sequence 13114 | AI435207 | EST | 4301566 |
| Sequence 13115 | AI446753 | EST | 4297414 |
| Sequence 13116 | AI458730 | EST | 4311309 |
| Sequence 13117 | AI458971 | EST | 4311550 |
| Sequence 13118 | AI459103 | EST | 4311682 |
| Sequence 13119 | AI459338 | EST | 4311917 |
| Sequence 13120 | AI468004 | EST | 4330094 |
| Sequence 13121 | AI469505 | EST | 4331595 |
| Sequence 13122 | AI479751 | EST | 4372919 |
| Sequence 13123 | AI480118 | EST | 4373286 |
| Sequence 13124 | AI492144 | EST | 4393147 |
| Sequence 13125 | AI497917 | EST | 4389899 |
| Sequence 13126 | AI499678 | EST | 4391660 |
| Sequence 13127 | AI500313 | EST | 4392295 |
| Sequence 13128 | AI554818 | EST | 4487181 |
| Sequence 13129 | AI559531 | EST | 4509736 |
| Sequence 13130 | AI560688 | EST | 4511029 |
| Sequence 13131 | AI566007 | EST | 4524459 |
| Sequence 13132 | AI567612 | EST | 4526064 |
| Sequence 13133 | AI568146 | EST | 4526720 |
| Sequence 13134 | AI568614 | EST | 4531988 |
| Sequence 13135 | AI572600 | EST | 4535974 |
| Sequence 13136 | AI581048 | EST | 4565424 |
| Sequence 13137 | AI582231 | EST | 4568128 |
| Sequence 13138 | AI588871 | EST | 4597919 |
| Sequence 13139 | AI597782 | EST | 4606830 |
| Sequence 13140 | AI610953 | EST | 4620120 |
| Sequence 13141 | AI611995 | EST | 4621162 |
| Sequence 13142 | AI619754 | EST | 4628880 |
| Sequence 13143 | AI627855 | EST | 4664655 |
| Sequence 13144 | AI631608 | EST | 4682938 |
| Sequence 13145 | AI631996 | EST | 4683326 |
| Sequence 13146 | AI632109 | EST | 4683439 |
| Sequence 13147 | AI632955 | EST | 4684285 |
| Sequence 13148 | AI636794 | EST | 4688124 |
| Sequence 13149 | AI653101 | EST | 4737080 |
| Sequence 13150 | AI653441 | EST | 4737420 |
| Sequence 13151 | AI655644 | EST | 4739623 |
| Sequence 13152 | AI659251 | EST | 4762821 |
| Sequence 13153 | AI659386 | EST | 4762956 |
| Sequence 13154 | AI669708 | EST | 4834482 |
| Sequence 13155 | AI678630 | EST | 4888812 |
| Sequence 13156 | AI681539 | EST | 4891721 |
| Sequence 13157 | AI683912 | EST | 4894094 |
| Sequence 13158 | AI684760 | EST | 4896054 |
| Sequence 13159 | AI685829 | EST | 4897123 |
| Sequence 13160 | AI688152 | EST | 4899446 |
| Sequence 13161 | AI688730 | EST | 4900024 |
| Sequence 13162 | AI690281 | EST | 4901575 |
| Sequence 13163 | AI692476 | EST | 4969816 |
| Sequence 13164 | AI692763 | EST | 4970103 |
| Sequence 13165 | AI694509 | EST | 4971849 |
| Sequence 13166 | AI695805 | EST | 4983705 |
| Sequence 13167 | AI703457 | EST | 4991357 |
| Sequence 13168 | AI741323 | EST | 5109611 |
| Sequence 13169 | AI743098 | EST | 5111386 |
| Sequence 13170 | AI744238 | EST | 5112526 |
| Sequence 13171 | AI744256 | EST | 5112544 |
| Sequence 13172 | AI744984 | EST | 5113272 |
| Sequence 13173 | AI749216 | EST | 5127480 |
| Sequence 13174 | AI752250 | EST | 5130514 |
| Sequence 13175 | AI753825 | EST | 5132177 |
| Sequence 13176 | AI760744 | EST | 5176411 |
| Sequence 13177 | AI762877 | EST | 5178544 |
| Sequence 13178 | AI766072 | EST | 5232581 |
| Sequence 13179 | AI769461 | EST | 5235970 |
| Sequence 13180 | AI792057 | EST | 5339773 |
| Sequence 13181 | AI796688 | EST | 5362151 |
| Sequence 13182 | AI802907 | EST | 5368368 |
| Sequence 13183 | AI803019 | EST | 5368491 |
| Sequence 13184 | AI803566 | EST | 5369026 |
| Sequence 13185 | AI806628 | EST | 5393194 |
| Sequence 13186 | AI811625 | EST | 5398191 |
| Sequence 13187 | AI815404 | EST | 5430950 |
| Sequence 13188 | AI815499 | EST | 5431045 |
| Sequence 13189 | AI816161 | EST | 5431707 |
| Sequence 13190 | AI819909 | EST | 5438988 |
| Sequence 13191 | AI824193 | EST | 5444864 |
| Sequence 13192 | AI826628 | EST | 5447299 |
| Sequence 13193 | AI859220 | EST | 5512836 |
| Sequence 13194 | AI863349 | EST | 5527456 |
| Sequence 13195 | AI866561 | EST | 5530668 |
| Sequence 13196 | AI868330 | EST | 5541346 |
| Sequence 13197 | AI872857 | EST | 5546906 |
| Sequence 13198 | AI889718 | EST | 5594882 |
| Sequence 13199 | AI905192 | EST | 6495579 |
| Sequence 13200 | AI912093 | EST | 5631948 |
| Sequence 13201 | AI921575 | EST | 5657539 |
| Sequence 13202 | AI922278 | EST | 5658242 |
| Sequence 13203 | AI925156 | EST | 5661120 |
| Sequence 13204 | AI928710 | EST | 5664674 |
| Sequence 13205 | AI932970 | EST | 5671707 |
| Sequence 13206 | AI939359 | EST | 5678229 |
| Sequence 13207 | AI951970 | EST | 5744280 |
| Sequence 13208 | AI952576 | EST | 5744886 |
| Sequence 13209 | AI954275 | EST | 5746585 |
| Sequence 13210 | AI962421 | EST | 5755134 |
| Sequence 13211 | AI962746 | EST | 5755459 |
| Sequence 13212 | AI968255 | EST | 5765073 |
| Sequence 13213 | AI970812 | EST | 5767638 |
| Sequence 13214 | AI991063 | EST | 5837966 |
| Sequence 13215 | AI992283 | EST | 5839188 |
| Sequence 13216 | AL036115 | EST | 5927646 |
| Sequence 13217 | AL036349 | EST | 5405930 |
| Sequence 13218 | AL036413 | EST | 5405969 |
| Sequence 13219 | AL037368 | EST | 5406772 |
| Sequence 13220 | AL038505 | EST | 5928459 |
| Sequence 13221 | AL040528 | EST | 5935315 |
| Sequence 13222 | AL040985 | EST | 5409929 |
| Sequence 13223 | AL042384 | EST | 5421723 |
| Sequence 13224 | AL044640 | EST | 5432855 |
| Sequence 13225 | AL048351 | EST | 5936451 |
| Sequence 13226 | AL134733 | EST | 6602920 |
| Sequence 13227 | AL157600 | EST | 7058001 |
| Sequence 13228 | AW004978 | EST | 5853682 |
| Sequence 13229 | AW014893 | EST | 5863650 |
| Sequence 13230 | AW020158 | EST | 5873698 |
| Sequence 13231 | AW027717 | EST | 5886473 |
| Sequence 13232 | AW051707 | EST | 5913989 |
| Sequence 13233 | AW069100 | EST | 6024098 |
| Sequence 13234 | AW069166 | EST | 6024252 |
| Sequence 13235 | AW069188 | EST | 6024274 |
| Sequence 13236 | AW070930 | EST | 6025928 |
| Sequence 13237 | AW083185 | EST | 6038337 |
| Sequence 13238 | AW084353 | EST | 6039505 |
| Sequence 13239 | AW086188 | EST | 6041262 |
| Sequence 13240 | AW088595 | EST | 6044412 |
| Sequence 13241 | AW089333 | EST | 6046677 |
| Sequence 13242 | AW118097 | EST | 6086681 |
| Sequence 13243 | AW136562 | EST | 6140695 |
| Sequence 13244 | AW148690 | EST | 6196586 |
| Sequence 13245 | AW151847 | EST | 6199832 |
| Sequence 13246 | AW166819 | EST | 6398344 |
| Sequence 13247 | AW167129 | EST | 6398654 |
| Sequence 13248 | AW168050 | EST | 6399575 |
| Sequence 13249 | AW178252 | EST | 6444289 |
| Sequence 13250 | AW188275 | EST | 6462711 |
| Sequence 13251 | AW188668 | EST | 6463028 |
| Sequence 13252 | AW190301 | EST | 6464781 |
| Sequence 13253 | AW192683 | EST | 6471382 |
| Sequence 13254 | AW194025 | EST | 6472756 |
| Sequence 13255 | AW194497 | EST | 6473323 |
| Sequence 13256 | AW195584 | EST | 6474734 |
| Sequence 13257 | AW196723 | EST | 6475953 |
| Sequence 13258 | AW204679 | EST | 6504151 |
| Sequence 13259 | AW207172 | EST | 6506668 |
| Sequence 13260 | AW243325 | EST | 6577165 |
| Sequence 13261 | AW263682 | EST | 6640498 |
| Sequence 13262 | AW290969 | EST | 6697605 |
| Sequence 13263 | AW292970 | EST | 6699606 |
| Sequence 13264 | AW295194 | EST | 6701830 |
| Sequence 13265 | AW297219 | EST | 6703855 |
| Sequence 13266 | AW301845 | EST | 6711522 |
| Sequence 13267 | AW302764 | EST | 6712444 |
| Sequence 13268 | AW364986 | EST | 6869636 |
| Sequence 13269 | AW365134 | EST | 6869784 |
| Sequence 13270 | AW391931 | EST | 6896590 |
| Sequence 13271 | AW402563 | EST | 6921261 |
| Sequence 13272 | AW405635 | EST | 6924692 |
| Sequence 13273 | AW410936 | EST | 6936477 |
| Sequence 13274 | AW411275 | EST | 6936816 |
| Sequence 13275 | AW444608 | EST | 6986370 |
| Sequence 13276 | AW451259 | EST | 6992035 |
| Sequence 13277 | AW474887 | EST | 7044993 |
| Sequence 13278 | AW578558 | EST | 7253607 |
| Sequence 13279 | F29317 | EST | 4814943 |
| Sequence 13280 | F34866 | EST | 4820492 |
| Sequence 13281 | F36899 | EST | 4822525 |
| Sequence 13282 | H17774 | EST | 884014 |
| Sequence 13283 | H54240 | EST | 994387 |
| Sequence 13284 | H56010 | EST | 1004654 |
| Sequence 13285 | H71857 | EST | 1043673 |
| Sequence 13286 | H78871 | EST | 1056960 |
| Sequence 13287 | H97996 | EST | 1118881 |
| Sequence 13288 | N20525 | EST | 1125480 |
| Sequence 13289 | N27572 | EST | 1142053 |
| Sequence 13290 | N48918 | EST | 1190084 |
| Sequence 13291 | N62491 | EST | 1210320 |
| Sequence 13292 | N95724 | EST | 1268009 |
| Sequence 13293 | R02774 | EST | 752510 |
| Sequence 13294 | R06525 | EST | 757145 |
| Sequence 13295 | R14391 | EST | 767467 |
| Sequence 13296 | R55374 | EST | 824669 |
| Sequence 13297 | T08334 | EST | 389362 |
| Sequence 13298 | T24823 | EST | 534448 |
| Sequence 13299 | T60891 | EST | 663928 |
| Sequence 13300 | T82957 | EST | 711245 |
| Sequence 13301 | T83814 | EST | 712102 |
| Sequence 13302 | W01580 | EST | 1273634 |
| Sequence 13303 | W04263 | EST | 1276162 |
| Sequence 13304 | W19343 | EST | 1295012 |
| Sequence 13305 | W23474 | EST | 1300308 |
| Sequence 13306 | W69300 | EST | 1378561 |
| Sequence 13307 | W86630 | EST | 1400227 |
| Sequence 13308 | V21239 | NUCPATENT | N/A |
| Sequence 13309 | V34331 | NUCPATENT | N/A |
| Sequence 13310 | V43615 | NUOPATENT | N/A |
| Sequence 13311 | V84583 | NUCPATENT | N/A |
| Sequence 13312 | X03841 | NUCPATENT | N/A |
| Sequence 13313 | X30393 | NUCPATENT | N/A |
| Sequence 13314 | X40045 | NUCPATENT | N/A |
| Sequence 13315 | X84983 | NUCPATENT | 1488593 |
| Sequence 13316 | X97975 | NUCPATENT | 1340080 |
| Sequence 13317 | X98010 | NUCPATENT | 1360094 |
| Sequence 13318 | Z14718 | NUCPATENT | 6383 |
| Sequence 13319 | Z40810 | NUCPATENT | 566550 |
| Sequence 13320 | Z41320 | NUCPATENT | 567459 |
| Sequence 13321 | Z41384 | NUCPATENT | 567528 |
| Sequence 13322 | AC02716 | PREPATNUC | N/A |
| Sequence 13323 | AC36924 | PREPATNUC | N/A |
| Sequence 13324 | AC36925 | PREPATNUC | N/A |
| Sequence 13325 | AC36946 | PREPATNUC | N/A |
| Sequence 13326 | AC37777 | PREPATNUC | N/A |
| Sequence 13327 | AC38546 | PREPATNUC | N/A |
| Sequence 13328 | AC39510 | PREPATNUC | N/A |
| Sequence 13329 | AC2759 | Genbank | 345130 |
| Sequence 13330 | AC6846 | Genbank | 490055 |
| Sequence 13331 | A14133 | Genbank | 490127 |
| Sequence 13332 | A16794 | Genbank | 512417 |
| Sequence 13333 | A16796 | Genbank | 579569 |
| Sequence 13334 | A61385 | Genbank | 3715797 |
| Sequence 13335 | AB000584 | Genbank | 1813326 |
| Sequence 13336 | AB001535 | Genbank | 3928755 |
| Sequence 13337 | AB002303 | Genbank | 2224550 |
| Sequence 13338 | AB002308 | Genbank | 6634012 |
| Sequence 13339 | AB002322 | Genbank | 6634014 |
| Sequence 13340 | AB002323 | Genbank | 2224590 |
| Sequence 13341 | AB002360 | Genbank | 2224664 |
| Sequence 13342 | AB002363 | Genbank | 2224670 |
| Sequence 13343 | AB002364 | Genbank | 2224672 |
| Sequence 13344 | AB002366 | Genbank | 2224676 |
| Sequence 13345 | AB002368 | Genbank | 2224680 |
| Sequence 13346 | AB002387 | Genbank | 2224718 |
| Sequence 13347 | AB002391 | Genbank | 6683696 |
| Sequence 13348 | AB002533 | Genbank | 1944124 |
| Sequence 13349 | AB003102 | Genbank | 1945608 |
| Sequence 13350 | AB003723 | Genbank | 2911141 |
| Sequence 13351 | AB004064 | Genbank | 6983841 |
| Sequence 13352 | AB004574 | Genbank | 3184394 |
| Sequence 13353 | AB004857 | Genbank | 2911111 |
| Sequence 13354 | AB006534 | Genbank | 2924619 |
| Sequence 13355 | AB006626 | Genbank | 6635126 |
| Sequence 13356 | AB007144 | Genbank | 2911155 |
| Sequence 13357 | AB007170 | Genbank | 3077765 |
| Sequence 13358 | AB007874 | Genbank | 2887448 |
| Sequence 13359 | AB007890 | Genbank | 6634032 |
| Sequence 13360 | AB007956 | Genbank | 3413930 |
| Sequence 13361 | AB011083 | Genbank | 3043545 |
| Sequence 13362 | AB011105 | Genbank | 3043589 |
| Sequence 13363 | AB011111 | Genbank | 3043601 |
| Sequence 13364 | AB011120 | Genbank | 3043619 |
| Sequence 13365 | AB011148 | Genbank | 3043675 |
| Sequence 13366 | AB011149 | Genbank | 3043677 |
| Sequence 13367 | AB011155 | Genbank | 3043689 |
| Sequence 13368 | AB014545 | Genbank | 3327103 |
| Sequence 13369 | AB014547 | Genbank | 3327107 |
| Sequence 13370 | AB014560 | Genbank | 3327133 |
| Sequence 13371 | AB014563 | Genbank | 3327139 |
| Sequence 13372 | AB014570 | Genbank | 3327153 |
| Sequence 13373 | AB014579 | Genbank | 3327171 |
| Sequence 13374 | AB014589 | Genbank | 3327191 |
| Sequence 13375 | AB014600 | Genbank | 3327213 |
| Sequence 13376 | AB015639 | Genbank | 5821139 |
| Sequence 13377 | AB015752 | Genbank | 3746106 |
| Sequence 13378 | AB017103 | Genbank | 3493334 |
| Sequence 13379 | AB017335 | Genbank | 3582440 |
| Sequence 13380 | AB017548 | Genbank | 3885363 |
| Sequence 13381 | AB017654 | Genbank | 6979002 |
| Sequence 13382 | AB017709 | Genbank | 3650435 |
| Sequence 13383 | AB018257 | Genbank | 3882148 |
| Sequence 13384 | AB018260 | Genbank | 3882154 |
| Sequence 13385 | AB018267 | Genbank | 3882168 |
| Sequence 13386 | AB018319 | Genbank | 3882272 |
| Sequence 13387 | AB018330 | Genbank | 3882294 |
| Sequence 13388 | AB018340 | Genbank | 3882314 |
| Sequence 13389 | AB018342 | Genbank | 3882318 |
| Sequence 13390 | AB018344 | Genbank | 3882322 |
| Sequence 13391 | AB018353 | Genbank | 3882340 |
| Sequence 13392 | AB019038 | Genbank | 6970469 |
| Sequence 13393 | AB019408 | Genbank | 4587126 |
| Sequence 13394 | AB019441 | Genbank | 4512311 |
| Sequence 13395 | AB019524 | Genbank | 4519939 |
| Sequence 13396 | AB019527 | Genbank | 3869126 |
| Sequence 13397 | AB019564 | Genbank | 3885367 |
| Sequence 13398 | AB020018 | Genbank | 6456471 |
| Sequence 13399 | AB020236 | Genbank | 4760601 |
| Sequence 13400 | AB020335 | Genbank | 6518494 |
| Sequence 13401 | AB020637 | Genbank | 4240148 |
| Sequence 13402 | AB020656 | Genbank | 4240186 |
| Sequence 13403 | AB020660 | Genbank | 4240194 |
| Sequence 13404 | AB020662 | Genbank | 4240198 |
| Sequence 13405 | AB020676 | Genbank | 4240226 |
| Sequence 13406 | AB020682 | Genbank | 4240238 |
| Sequence 13407 | AB020694 | Genbank | 4240262 |
| Sequence 13408 | AB020705 | Genbank | 4240284 |
| Sequence 13409 | AB020711 | Genbank | 4240296 |
| Sequence 13410 | AB020859 | Genbank | 4003379 |
| Sequence 13411 | AB020868 | Genbank | 4003388 |
| Sequence 13412 | AB020982 | Genbank | 4417209 |
| Sequence 13413 | AB021470 | Genbank | 4586521 |
| Sequence 13414 | AB021663 | Genbank | 4996450 |
| Sequence 13415 | AB022435 | Genbank | 4996607 |
| Sequence 13416 | AB022537 | Genbank | 6942217 |
| Sequence 13417 | AB022785 | Genbank | 4210446 |
| Sequence 13418 | AB023207 | Genbank | 4589623 |
| Sequence 13419 | AB023208 | Genbank | 4589625 |
| Sequence 13420 | AB023224 | Genbank | 4589657 |
| Sequence 13421 | AB023691 | Genbank | 6519222 |
| Sequence 13422 | AB024334 | Genbank | 6016837 |
| Sequence 13423 | AB025355 | Genbank | 5103146 |
| Sequence 13424 | AB025959 | Genbank | 6520072 |
| Sequence 13425 | AB027466 | Genbank | 6172220 |
| Sequence 13426 | AB028624 | Genbank | 5103045 |
| Sequence 13427 | AB028893 | Genbank | 6552364 |
| Sequence 13428 | AB029020 | Genbank | 5689530 |
| Sequence 13429 | AB029331 | Genbank | 5295999 |
| Sequence 13430 | AB030181 | Genbank | 6730949 |
| Sequence 13431 | AB030905 | Genbank | 5732186 |
| Sequence 13432 | AB032951 | Genbank | 6329748 |
| Sequence 13433 | AB032957 | Genbank | 6329818 |
| Sequence 13434 | AB032962 | Genbank | 6382013 |
| Sequence 13435 | AB032969 | Genbank | 6329965 |
| Sequence 13436 | AB033007 | Genbank | 6330242 |
| Sequence 13437 | AB033018 | Genbank | 6330350 |
| Sequence 13438 | AB033020 | Genbank | 6330364 |
| Sequence 13439 | AB033050 | Genbank | 6330623 |
| Sequence 13440 | AB033067 | Genbank | 6330797 |
| Sequence 13441 | AB033069 | Genbank | 6330811 |
| Sequence 13442 | AB033074 | Genbank | 6330846 |
| Sequence 13443 | AB033076 | Genbank | 6330860 |
| Sequence 13444 | AB033079 | Genbank | 6382025 |
| Sequence 13445 | AB033096 | Genbank | 6331225 |
| Sequence 13446 | AB034205 | Genbank | 6899845 |
| Sequence 13447 | AB037763 | Genbank | 7243064 |
| Sequence 13448 | AB037767 | Genbank | 7243072 |
| Sequence 13449 | AB037807 | Genbank | 7243152 |
| Sequence 13450 | AB037813 | Genbank | 7243164 |
| Sequence 13451 | AC000015 | Genbank | 4263638 |
| Sequence 13452 | AC000025 | Genbank | 4417318 |
| Sequence 13453 | AC000046 | Genbank | 4731059 |
| Sequence 13454 | AC000093 | Genbank | 4678240 |
| Sequence 13455 | AC000120 | Genbank | 1809224 |
| Sequence 13456 | AC000353 | Genbank | 6970735 |
| Sequence 13457 | AC000378 | Genbank | 2270906 |
| Sequence 13458 | AC000394 | Genbank | 2133911 |
| Sequence 13459 | AC001226 | Genbank | 2133862 |
| Sequence 13460 | AC001234 | Genbank | 4028939 |
| Sequence 13461 | AC002038 | Genbank | 2226439 |
| Sequence 13462 | AC002039 | Genbank | 2342716 |
| Sequence 13463 | AC002041 | Genbank | 2576343 |
| Sequence 13464 | AC002064 | Genbank | 2076723 |
| Sequence 13465 | AC002081 | Genbank | 2078453 |
| Sequence 13466 | AC002094 | Genbank | 2155224 |
| Sequence 13467 | AC002096 | Genbank | 2599243 |
| Sequence 13468 | AC002347 | Genbank | 2828783 |
| Sequence 13469 | AC002350 | Genbank | 2961443 |
| Sequence 13470 | AC002365 | Genbank | 2358015 |
| Sequence 13471 | AC002366 | Genbank | 2739349 |
| Sequence 13472 | AC002394 | Genbank | 2815550 |
| Sequence 13473 | AC002395 | Genbank | 3540146 |
| Sequence 13474 | AC002398 | Genbank | 2529398 |
| Sequence 13475 | AC002401 | Genbank | 2599240 |
| Sequence 13476 | AC002487 | Genbank | 2341013 |
| Sequence 13477 | AC002522 | Genbank | 7124014 |
| Sequence 13478 | AC002542 | Genbank | 2393733 |
| Sequence 13479 | AC002544 | Genbank | 3337382 |
| Sequence 13480 | AC002558 | Genbank | 2580474 |
| Sequence 13481 | AC003004 | Genbank | 2522284 |
| Sequence 13482 | AC003007 | Genbank | 2911728 |
| Sequence 13483 | AC003026 | Genbank | 2815549 |
| Sequence 13484 | AC003029 | Genbank | 9965520 |
| Sequence 13485 | AC003037 | Genbank | 2920805 |
| Sequence 13486 | AC003041 | Genbank | 3264572 |
| Sequence 13487 | AC003046 | Genbank | 10043285 |
| Sequence 13488 | AC003070 | Genbank | 2865212 |
| Sequence 13489 | AC003103 | Genbank | 2842782 |
| Sequence 13490 | AC003104 | Genbank | 3253131 |
| Sequence 13491 | AC003663 | Genbank | 3097871 |
| Sequence 13492 | AC003682 | Genbank | 3264845 |
| Sequence 13493 | AC003688 | Genbank | 3789719 |
| Sequence 13494 | AC003963 | Genbank | 2995604 |
| Sequence 13495 | AC003989 | Genbank | 2772538 |
| Sequence 13496 | AC004006 | Genbank | 2781390 |
| Sequence 13497 | AC004013 | Genbank | 2781380 |
| Sequence 13498 | AC004073 | Genbank | 2822136 |
| Sequence 13499 | AC004084 | Genbank | 2822156 |
| Sequence 13500 | AC004106 | Genbank | 3150276 |
| Sequence 13501 | AC004126 | Genbank | 3242744 |
| Sequence 13502 | AC004142 | Genbank | 2880078 |
| Sequence 13503 | AC004148 | Genbank | 3482960 |
| Sequence 13504 | AC004152 | Genbank | 2894631 |
| Sequence 13505 | AC004156 | Genbank | 2896799 |
| Sequence 13506 | AC004158 | Genbank | 2982170 |
| Sequence 13507 | AC004221 | Genbank | 9665053 |
| Sequence 13508 | AC004222 | Genbank | 3108042 |
| Sequence 13509 | AC004223 | Genbank | 3253129 |
| Sequence 13510 | AC004227 | Genbank | 2911718 |
| Sequence 13511 | AC004228 | Genbank | 4263838 |
| Sequence 13512 | AC004233 | Genbank | 2914673 |
| Sequence 13513 | AC004236 | Genbank | 2914668 |
| Sequence 13514 | AC004258 | Genbank | 2924759 |
| Sequence 13515 | AC004263 | Genbank | 2935616 |
| Sequence 13516 | AC004381 | Genbank | 2982169 |
| Sequence 13517 | AC004382 | Genbank | 3252819 |
| Sequence 13518 | AC004388 | Genbank | 3046271 |
| Sequence 13519 | AC004408 | Genbank | 3873185 |
| Sequence 13520 | AC004466 | Genbank | 3617739 |
| Sequence 13521 | AC004477 | Genbank | 3688107 |
| Sequence 13522 | AC004494 | Genbank | 2996647 |
| Sequence 13523 | AC004507 | Genbank | 2996632 |
| Sequence 13524 | AC004520 | Genbank | 3004572 |
| Sequence 13525 | AC004522 | Genbank | 3006227 |
| Sequence 13526 | AC004527 | Genbank | 4760422 |
| Sequence 13527 | AC004531 | Genbank | 3337392 |
| Sequence 13528 | AC004539 | Genbank | 3041851 |
| Sequence 13529 | AC004582 | Genbank | 3337307 |
| Sequence 13530 | AC004584 | Genbank | 3417305 |
| Sequence 13531 | AC004585 | Genbank | 3212882 |
| Sequence 13532 | AC004587 | Genbank | 3150014 |
| Sequence 13533 | AC004600 | Genbank | 4388746 |
| Sequence 13534 | AC004601 | Genbank | 3395446 |
| Sequence 13535 | AC004611 | Genbank | 3080643 |
| Sequence 13536 | AC004617 | Genbank | 3738114 |
| Sequence 13537 | AC004622 | Genbank | 3090700 |
| Sequence 13538 | AC004634 | Genbank | 3094993 |
| Sequence 13539 | AC004677 | Genbank | 3126881 |
| Sequence 13540 | AC0004686 | Genbank | 3688105 |
| Sequence 13541 | AC004760 | Genbank | 3168626 |
| Sequence 13542 | AC004765 | Genbank | 4731046 |
| Sequence 13543 | AC004770 | Genbank | 3212836 |
| Sequence 13544 | AC004798 | Genbank | 3184497 |
| Sequence 13545 | AC004801 | Genbank | 4204244 |
| Sequence 13546 | AC004812 | Genbank | 3970970 |
| Sequence 13547 | AC004813 | Genbank | 4926912 |
| Sequence 13548 | AC004832 | Genbank | 6624129 |
| Sequence 13549 | AC004835 | Genbank | 4508154 |
| Sequence 13550 | AC004842 | Genbank | 4753286 |
| Sequence 13551 | AC004846 | Genbank | 7243869 |
| Sequence 13552 | AC004854 | Genbank | 4827328 |
| Sequence 13553 | AC004876 | Genbank | 4508148 |
| Sequence 13554 | AC004890 | Genbank | 4508146 |
| Sequence 13555 | AC004904 | Genbank | 4156180 |
| Sequence 13556 | AC004908 | Genbank | 4156179 |
| Sequence 13557 | AC004938 | Genbank | 3213059 |
| Sequence 13558 | AC004941 | Genbank | 4454517 |
| Sequence 13559 | AC004950 | Genbank | 4753269 |
| Sequence 13560 | AC004955 | Genbank | 4753267 |
| Sequence 13561 | AC004969 | Genbank | 4156159 |
| Sequence 13562 | AC004982 | Genbank | 3419846 |
| Sequence 13563 | AC004983 | Genbank | 4309885 |
| Sequence 13564 | AC004985 | Genbank | 5708490 |
| Sequence 13565 | AC004987 | Genbank | 3213010 |
| Sequence 13566 | AC004990 | Genbank | 3924668 |
| Sequence 13567 | AC004999 | Genbank | 3970963 |
| Sequence 13568 | AC005005 | Genbank | 4156153 |
| Sequence 13569 | AC005007 | Genbank | 4156151 |
| Sequence 13570 | AC005021 | Genbank | 3694657 |
| Sequence 13571 | AC005031 | Genbank | 3688109 |
| Sequence 13572 | AC005037 | Genbank | 4827310 |
| Sequence 13573 | AC005041 | Genbank | 4508118 |
| Sequence 13574 | AC005042 | Genbank | 4156138 |
| Sequence 13575 | AC005046 | Genbank | 6094632 |
| Sequence 13576 | AC005076 | Genbank | 4508110 |
| Sequence 13577 | AC005082 | Genbank | 5836159 |
| Sequence 13578 | AC005084 | Genbank | 3659503 |
| Sequence 13579 | AC005088 | Genbank | 4753221 |
| Sequence 13580 | AC005095 | Genbank | 4753220 |
| Sequence 13581 | AC005104 | Genbank | 4218027 |
| Sequence 13582 | AC005166 | Genbank | 3242769 |
| Sequence 13583 | AC005187 | Genbank | 3522927 |
| Sequence 13584 | AC005189 | Genbank | 3264580 |
| Sequence 13585 | AC005193 | Genbank | 3309101 |
| Sequence 13586 | AC005203 | Genbank | 3273381 |
| Sequence 13587 | AC005212 | Genbank | 3287444 |
| Sequence 13588 | AC005251 | Genbank | 3287718 |
| Sequence 13589 | AC005253 | Genbank | 3288886 |
| Sequence 13590 | AC005261 | Genbank | 3289984 |
| Sequence 13591 | AC005262 | Genbank | 3289981 |
| Sequence 13592 | AC005300 | Genbank | 6492490 |
| Sequence 13593 | AC005318 | Genbank | 3885345 |
| Sequence 13594 | AC005332 | Genbank | 3659494 |
| Sequence 13595 | AC005360 | Genbank | 3367514 |
| Sequence 13596 | AC005363 | Genbank | 3367511 |
| Sequence 13597 | AC005366 | Genbank | 3367508 |
| Sequence 13598 | AC005387 | Genbank | 3395417 |
| Sequence 13599 | AC005391 | Genbank | 3399673 |
| Sequence 13600 | AC005406 | Genbank | 4580403 |
| Sequence 13601 | AC005409 | Genbank | 4249432 |
| Sequence 13602 | AC005410 | Genbank | 3450753 |
| Sequence 13603 | AC005484 | Genbank | 5091654 |
| Sequence 13604 | AC005488 | Genbank | 5836194 |
| Sequence 13605 | AC005509 | Genbank | 4176355 |
| Sequence 13606 | AC005531 | Genbank | 4153875 |
| Sequence 13607 | AC005550 | Genbank | 3478668 |
| Sequence 13608 | AC005562 | Genbank | 4153858 |
| Sequence 13609 | AC005613 | Genbank | 3540152 |
| Sequence 13610 | AC005627 | Genbank | 7243867 |
| Sequence 13611 | AC005629 | Genbank | 7243877 |
| Sequence 13612 | AC005630 | Genbank | 4159882 |
| Sequence 13613 | AC005661 | Genbank | 3818348 |
| Sequence 13614 | AC005668 | Genbank | 3641771 |
| Sequence 13615 | AC005669 | Genbank | 3702356 |
| Sequence 13616 | AC005726 | Genbank | 3810672 |
| Sequence 13617 | AC005730 | Genbank | 3779042 |
| Sequence 13618 | AC005740 | Genbank | 3687210 |
| Sequence 13619 | AC005768 | Genbank | 6598827 |
| Sequence 13620 | AC005785 | Genbank | 3702290 |
| Sequence 13621 | AC005789 | Genbank | 3702281 |
| Sequence 13622 | AC005828 | Genbank | 3789713 |
| Sequence 13623 | AC005829 | Genbank | 3873300 |
| Sequence 13624 | AC005837 | Genbank | 3849820 |
| Sequence 13625 | AC005838 | Genbank | 3947427 |
| Sequence 13626 | AC005839 | Genbank | 4079626 |
| Sequence 13627 | AC005841 | Genbank | 4731044 |
| Sequence 13628 | AC005876 | Genbank | 6249672 |
| Sequence 13629 | AC005880 | Genbank | 6249667 |
| Sequence 13630 | AC005899 | Genbank | 3935221 |
| Sequence 13631 | AC005912 | Genbank | 4165005 |
| Sequence 13632 | AC005923 | Genbank | 4309927 |
| Sequence 13633 | AC005924 | Genbank | 4309926 |
| Sequence 13634 | AC005969 | Genbank | 4309946 |
| Sequence 13635 | AC005998 | Genbank | 5649387 |
| Sequence 13636 | AC006009 | Genbank | 4753278 |
| Sequence 13637 | AC006011 | Genbank | 4508143 |
| Sequence 13638 | AC006017 | Genbank | 4508141 |
| Sequence 13639 | AC006023 | Genbank | 4753260 |
| Sequence 13640 | AC006026 | Genbank | 6042101 |
| Sequence 13641 | AC006027 | Genbank | 4156149 |
| Sequence 13642 | AC006050 | Genbank | 4079627 |
| Sequence 13643 | AC006057 | Genbank | 4731048 |
| Sequence 13644 | AC006064 | Genbank | 4572650 |
| Sequence 13645 | AC006084 | Genbank | 3953485 |
| Sequence 13646 | AC006088 | Genbank | 4204701 |
| Sequence 13647 | AC006129 | Genbank | 3970933 |
| Sequence 13648 | AC006137 | Genbank | 9887710 |
| Sequence 13649 | AC006141 | Genbank | 4580411 |
| Sequence 13650 | AC006153 | Genbank | 4309917 |
| Sequence 13651 | AC006160 | Genbank | 5701616 |
| Sequence 13652 | AC006205 | Genbank | 5815508 |
| Sequence 13653 | AC006206 | Genbank | 4309925 |
| Sequence 13654 | AC006207 | Genbank | 4309924 |
| Sequence 13655 | AC006238 | Genbank | 4204704 |
| Sequence 13656 | AC006251 | Genbank | 4309945 |
| Sequence 13657 | AC006254 | Genbank | 4726099 |
| Sequence 13658 | AC006255 | Genbank | 5306223 |
| Sequence 13659 | AC006257 | Genbank | 4092478 |
| Sequence 13660 | AC006273 | Genbank | 4096060 |
| Sequence 13661 | AC006347 | Genbank | 4309919 |
| Sequence 13662 | AC006349 | Genbank | 7243873 |
| Sequence 13663 | AC006409 | Genbank | 4454486 |
| Sequence 13664 | AC006430 | Genbank | 6453929 |
| Sequence 13665 | AC006449 | Genbank | 6102666 |
| Sequence 13666 | AC006450 | Genbank | 6997336 |
| Sequence 13667 | AC006475 | Genbank | 4753283 |
| Sequence 13668 | AC006480 | Genbank | 6289252 |
| Sequence 13669 | AC006487 | Genbank | 4314418 |
| Sequence 13670 | AC006504 | Genbank | 4220442 |
| Sequence 13871 | AC006509 | Genbank | 5815507 |
| Sequence 13672 | AC006512 | Genbank | 4926863 |
| Sequence 13673 | AC006518 | Genbank | 4713939 |
| Sequence 13674 | AC006536 | Genbank | 4235135 |
| Sequence 13675 | AC006539 | Genbank | 4235141 |
| Sequence 13676 | AC006561 | Genbank | 4558534 |
| Sequence 13677 | AC006581 | Genbank | 4914350 |
| Sequence 13678 | AC006597 | Genbank | 4512725 |
| Sequence 13679 | AC006930 | Genbank | 4309680 |
| Sequence 13680 | AC006973 | Genbank | 4699968 |
| Sequence 13681 | AC006987 | Genbank | 5123989 |
| Sequence 13682 | AC007014 | Genbank | 4371263 |
| Sequence 13683 | AC007029 | Genbank | 7243872 |
| Sequence 13684 | AC007038 | Genbank | 5708475 |
| Sequence 13685 | AC007041 | Genbank | 5708471 |
| Sequence 13686 | AC007050 | Genbank | 6456174 |
| Sequence 13687 | AC007055 | Genbank | 4885691 |
| Sequence 13688 | AC007057 | Genbank | 4680440 |
| Sequence 13889 | AC007114 | Genbank | 4581193 |
| Sequence 13690 | AC007130 | Genbank | 5001538 |
| Sequence 13691 | AC007172 | Genbank | 4731066 |
| Sequence 13692 | AC007204 | Genbank | 4559317 |
| Sequence 13893 | AC007207 | Genbank | 6466489 |
| Sequence 13694 | AC007216 | Genbank | 6806842 |
| Sequence 13695 | AC007225 | Genbank | 6715703 |
| Sequence 13696 | AC007229 | Genbank | 4567173 |
| Sequence 13697 | AC007239 | Genbank | 5523828 |
| Sequence 13698 | AC007252 | Genbank | 5091646 |
| Sequence 13699 | AC007314 | Genbank | 4662678 |
| Sequence 13700 | AC007324 | Genbank | 7923342 |
| Sequence 13701 | AC007327 | Genbank | 4587630 |
| Sequence 13702 | AC007390 | Genbank | 6358845 |
| Sequence 13703 | AC007393 | Genbank | 5306300 |
| Sequence 13704 | AC007446 | Genbank | 5069495 |
| Sequence 13705 | AC007528 | Genbank | 5263309 |
| Sequence 13706 | AC007536 | Genbank | 5917929 |
| Sequence 13707 | AC007541 | Genbank | 4982536 |
| Sequence 13708 | AC007546 | Genbank | 5668756 |
| Sequence 13709 | AC007551 | Genbank | 4809347 |
| Sequence 13710 | AC007566 | Genbank | 4835815 |
| Sequence 13711 | AC007766 | Genbank | 5030437 |
| Sequence 13712 | AC007848 | Genbank | 5923645 |
| Sequence 13713 | AC007860 | Genbank | 5649179 |
| Sequence 13714 | AC007868 | Genbank | 6041758 |
| Sequence 13715 | AC007934 | Genbank | 6721208 |
| Sequence 13716 | AC008009 | Genbank | 5668757 |
| Sequence 13717 | AC008018 | Genbank | 7958973 |
| Sequence 13718 | AC008102 | Genbank | 9797815 |
| Sequence 13719 | AC008115 | Genbank | 5801654 |
| Sequence 13720 | AC008123 | Genbank | 6006046 |
| Sequence 13721 | AC008151 | Genbank | 5629925 |
| Sequence 13722 | AC008498 | Genbank | 6850299 |
| Sequence 13723 | AC008545 | Genbank | 6721145 |
| Sequence 13724 | AC008583 | Genbank | 7158894 |
| Sequence 13725 | AC008893 | Genbank | 7259691 |
| Sequence 13726 | AC008981 | Genbank | 6721142 |
| Sequence 13727 | AC009298 | Genbank | 7243882 |
| Sequence 13728 | AC009363 | Genbank | 6648145 |
| Sequence 13729 | AC009509 | Genbank | 6492473 |
| Sequence 13730 | AC009946 | Genbank | 6604542 |
| Sequence 13731 | AC010077 | Genbank | 5870275 |
| Sequence 13732 | AC010168 | Genbank | 6855156 |
| Sequence 13733 | AC010197 | Genbank | 6143841 |
| Sequence 13734 | AC010436 | Genbank | 6938861 |
| Sequence 13735 | AC010525 | Genbank | 7248932 |
| Sequence 13736 | AC010582 | Genbank | 6721135 |
| Sequence 13737 | AC010722 | Genbank | 6587939 |
| Sequence 13738 | AC011331 | Genbank | 9929686 |
| Sequence 13739 | AC011599 | Genbank | 7113871 |
| Sequence 13740 | AC012085 | Genbank | 6634755 |
| Sequence 13741 | AC012087 | Genbank | 7114465 |
| Sequence 13742 | AC016831 | Genbank | 6539289 |
| Sequence 13743 | AC016941 | Genbank | 8567654 |
| Sequence 13744 | AC020663 | Genbank | 6682593 |
| Sequence 13745 | AC021049 | Genbank | 7248920 |
| Sequence 13746 | AF000982 | Genbank | 2580549 |
| Sequence 13747 | AF001549 | Genbank | 3355302 |
| Sequence 13748 | AF001552 | Genbank | 2339976 |
| Sequence 13749 | AF002668 | Genbank | 2232173 |
| Sequence 13750 | AF004162 | Genbank | 3046385 |
| Sequence 13751 | AF004337 | Genbank | 2245557 |
| Sequence 13752 | AF004338 | Genbank | 2245558 |
| Sequence 13753 | AF004339 | Genbank | 2245559 |
| Sequence 13754 | AF004342 | Genbank | 2245565 |
| Sequence 13755 | AF006513 | Genbank | 2645428 |
| Sequence 13756 | AF006621 | Genbank | 7629276 |
| Sequence 13757 | AF007134 | Genbank | 2852608 |
| Sequence 13758 | AF007146 | Genbank | 2852624 |
| Sequence 13759 | AF007189 | Genbank | 2459927 |
| Sequence 13760 | AF007544 | Genbank | 2970122 |
| Sequence 13761 | AF007872 | Genbank | 2358280 |
| Sequence 13762 | AF007875 | Genbank | 2258417 |
| Sequence 13763 | AF008303 | Genbank | 2580619 |
| Sequence 13764 | AF009242 | Genbank | 2338289 |
| Sequence 13765 | AF010313 | Genbank | 6468761 |
| Sequence 13766 | AF011889 | Genbank | 2335186 |
| Sequence 13767 | AF013488 | Genbank | 2343184 |
| Sequence 13768 | AF013758 | Genbank | 3046899 |
| Sequence 13769 | AF013970 | Genbank | 2801421 |
| Sequence 13770 | AF014398 | Genbank | 8148061 |
| Sequence 13771 | AF015812 | Genbank | 2599359 |
| Sequence 13772 | AF017790 | Genbank | 2501872 |
| Sequence 13773 | AF020352 | Genbank | 2655054 |
| Sequence 13774 | AF020782 | Genbank | 2522480 |
| Sequence 13775 | AF021819 | Genbank | 2460317 |
| Sequence 13776 | AF022211 | Genbank | 2465723 |
| Sequence 13777 | AF022728 | Genbank | 2935182 |
| Sequence 13778 | AF026802 | Genbank | 5712085 |
| Sequence 13779 | AF026852 | Genbank | 3599965 |
| Sequence 13780 | AF027205 | Genbank | 2598967 |
| Sequence 13781 | AF028832 | Genbank | 3287488 |
| Sequence 13782 | AF029750 | Genbank | 2587057 |
| Sequence 13783 | AF029786 | Genbank | 3403166 |
| Sequence 13784 | AF031379 | Genbank | 4894208 |
| Sequence 13785 | AF035286 | Genbank | 2661038 |
| Sequence 13786 | AF035296 | Genbank | 2661052 |
| Sequence 13787 | AF035304 | Genbank | 2661064 |
| Sequence 13788 | AF035840 | Genbank | 3800739 |
| Sequence 13789 | AF036715 | Genbank | 4007385 |
| Sequence 13790 | AF036718 | Genbank | 2708629 |
| Sequence 13791 | AF037447 | Genbank | 6466790 |
| Sequence 13792 | AF038200 | Genbank | 2795921 |
| Sequence 13793 | AF038406 | Genbank | 3523122 |
| Sequence 13794 | AF039019 | Genbank | 2828109 |
| Sequence 13795 | AF039575 | Genbank | 2773157 |
| Sequence 13796 | AF039594 | Genbank | 3366667 |
| Sequence 13797 | AF040628 | Genbank | 3869269 |
| Sequence 13798 | AF042836 | Genbank | 3776241 |
| Sequence 13799 | AF043105 | Genbank | 3169541 |
| Sequence 13800 | AF044956 | Genbank | 5326827 |
| Sequence 13801 | AF044957 | Genbank | 4164445 |
| Sequence 13802 | AF044959 | Genbank | 3348136 |
| Sequence 13803 | AF045447 | Genbank | 2865655 |
| Sequence 13804 | AF047184 | Genbank | 2909859 |
| Sequence 13805 | AF047185 | Genbank | 2909861 |
| Sequence 13806 | AF047438 | Genbank | 3335131 |
| Sequence 13807 | AF047440 | Genbank | 3335135 |
| Sequence 13808 | AF047472 | Genbank | 2921872 |
| Sequence 13809 | AF050638 | Genbank | 5326819 |
| Sequence 13810 | AF050639 | Genbank | 4164453 |
| Sequence 13811 | AF052092 | Genbank | 3360398 |
| Sequence 13812 | AF052129 | Genbank | 3360438 |
| Sequence 13813 | AF052164 | Genbank | 3360475 |
| Sequence 13814 | AF052182 | Genbank | 3360494 |
| Sequence 13815 | AF052573 | Genbank | 3510694 |
| Sequence 13816 | AF052578 | Genbank | 2967847 |
| Sequence 13817 | AF053356 | Genbank | 3135305 |
| Sequence 13818 | AF054183 | Genbank | 4092053 |
| Sequence 13819 | AF054990 | Genbank | 3005703 |
| Sequence 13820 | AF055066 | Genbank | 3273727 |
| Sequence 13821 | AF055474 | Genbank | nil |
| Sequence 13822 | AF055584 | Genbank | 3649607 |
| Sequence 13823 | AF057356 | Genbank | 3063652 |
| Sequence 13824 | AF057735 | Genbank | 4165042 |
| Sequence 13825 | AF058293 | Genbank | 3047377 |
| Sequence 13826 | AF058988 | Genbank | 3300089 |
| Sequence 13827 | AF059527 | Genbank | 4454661 |
| Sequence 13828 | AF060798 | Genbank | 3372665 |
| Sequence 13829 | AF061737 | Genbank | 4335938 |
| Sequence 13830 | AF062077 | Genbank | 3641524 |
| Sequence 13831 | AF064019 | Genbank | 3347856 |
| Sequence 13832 | AF064861 | Genbank | 3171154 |
| Sequence 13833 | AF067139 | Genbank | 3337440 |
| Sequence 13834 | AF068302 | Genbank | 4584876 |
| Sequence 13835 | AF068754 | Genbank | 3283408 |
| Sequence 13836 | AF068755 | Genbank | 4321979 |
| Sequence 13837 | AF069378 | Genbank | 3982733 |
| Sequence 13838 | AF069532 | Genbank | 3892190 |
| Sequence 13839 | AF070523 | Genbank | 3764088 |
| Sequence 13840 | AF070539 | Genbank | 3387896 |
| Sequence 13841 | AF070556 | Genbank | 3387921 |
| Sequence 13842 | AF070630 | Genbank | 3283898 |
| Sequence 13843 | AF070635 | Genbank | 3283905 |
| Sequence 13844 | AF070638 | Genbank | 3283909 |
| Sequence 13545 | AF070646 | Genbank | 3283920 |
| Sequence 13846 | AF070649 | Genbank | 3283923 |
| Sequence 13847 | AF070655 | Genbank | 4454685 |
| Sequence 13848 | AF070659 | Genbank | 4454693 |
| Sequence 13849 | AF070669 | Genbank | 4454713 |
| Sequence 13850 | AF070673 | Genbank | 3978241 |
| Sequence 13851 | AF072097 | Genbank | 5725511 |
| Sequence 13852 | AF073298 | Genbank | 3641537 |
| Sequence 13853 | AF075061 | Genbank | 3377602 |
| Sequence 13854 | AF077030 | Genbank | 4689107 |
| Sequence 13855 | AF077036 | Genbank | 4689119 |
| Sequence 13856 | AF077043 | Genbank | 4689133 |
| Sequence 13857 | AF077044 | Genbank | 4689135 |
| Sequence 13858 | AF077052 | Genbank | 4689151 |
| Sequence 13859 | AF077200 | Genbank | 4679013 |
| Sequence 13860 | AF077202 | Genbank | 4679017 |
| Sequence 13861 | AF078847 | Genbank | 5531808 |
| Sequence 13862 | AF078850 | Genbank | 5531814 |
| Sequence 13863 | AF082283 | Genbank | 4092066 |
| Sequence 13864 | AF082513 | Genbank | 3649656 |
| Sequence 13865 | AF083441 | Genbank | 5813822 |
| Sequence 13866 | AF085844 | Genbank | 3483158 |
| Sequence 13867 | AF086210 | Genbank | 3483555 |
| Sequence 13868 | AF086329 | Genbank | 3483674 |
| Sequence 13869 | AF086337 | Genbank | 3483682 |
| Sequence 13870 | AF086378 | Genbank | 3483723 |
| Sequence 13871 | AF086490 | Genbank | 3483835 |
| Sequence 13872 | AF086557 | Genbank | 3483902 |
| Sequence 13873 | AF086564 | Genbank | 3483909 |
| Sequence 13874 | AF086628 | Genbank | 4240457 |
| Sequence 13875 | AF087020 | Genbank | 3851144 |
| Sequence 13876 | AF087160 | Genbank | 3869380 |
| Sequence 13877 | AF087959 | Genbank | 3523165 |
| Sequence 13878 | AF088060 | Genbank | 3523266 |
| Sequence 13879 | AF088071 | Genbank | 3523277 |
| Sequence 13880 | AF090095 | Genbank | 4063630 |
| Sequence 13881 | AF090947 | Genbank | 6690255 |
| Sequence 13882 | AF091075 | Genbank | 3859987 |
| Sequence 13883 | AF092094 | Genbank | 4426606 |
| Sequence 13884 | AF092124 | Genbank | 3659900 |
| Sequence 13885 | AF092128 | Genbank | 5138905 |
| Sequence 13886 | AF092130 | Genbank | 5138909 |
| Sequence 13887 | AF092135 | Genbank | 5138919 |
| Sequence 13888 | AF097026 | Genbank | 6409120 |
| Sequence 13889 | AF097514 | Genbank | 4808600 |
| Sequence 13890 | AF098162 | Genbank | 3929582 |
| Sequence 13891 | AF100615 | Genbank | 4808624 |
| Sequence 13892 | AF100616 | Genbank | 4808626 |
| Sequence 13893 | AF100748 | Genbank | 5410281 |
| Sequence 13894 | AF100757 | Genbank | 5410299 |
| Sequence 13895 | AF100760 | Genbank | 5410305 |
| Sequence 13896 | AF102803 | Genbank | 4092759 |
| Sequence 13897 | AF104233 | Genbank | 5880481 |
| Sequence 13898 | AF104913 | Genbank | 3941723 |
| Sequence 13899 | AF106622 | Genbank | 4378528 |
| Sequence 13900 | AF106681 | Genbank | 5410327 |
| Sequence 13901 | AF109134 | Genbank | 4139227 |
| Sequence 13902 | AF109718 | Genbank | 3978547 |
| Sequence 13903 | AF110460 | Genbank | 4324698 |
| Sequence 13904 | AF111847 | Genbank | 7211441 |
| Sequence 13905 | AF112213 | Genbank | 6563213 |
| Sequence 13906 | AF112222 | Genbank | 6563229 |
| Sequence 13907 | AF112227 | Genbank | 4545218 |
| Sequence 13908 | AF113011 | Genbank | 6642745 |
| Sequence 13909 | AF113016 | Genbank | 6642755 |
| Sequence 13910 | AF113141 | Genbank | 4512031 |
| Sequence 13911 | AF113535 | Genbank | 6523824 |
| Sequence 13912 | AF113540 | Genbank | 6523834 |
| Sequence 13913 | AF113615 | Genbank | 5106955 |
| Sequence 13914 | AF113680 | Genbank | 6855605 |
| Sequence 13915 | AF113690 | Genbank | 6855619 |
| Sequence 13916 | AF113700 | Genbank | 6855634 |
| Sequence 13917 | AF113701 | Genbank | 6855635 |
| Sequence 13918 | AF115402 | Genbank | 4559272 |
| Sequence 13919 | AF116910 | Genbank | 4768837 |
| Sequence 13920 | AF117235 | Genbank | 6563243 |
| Sequence 13921 | AF117888 | Genbank | 5732617 |
| Sequence 13922 | AF118084 | Genbank | 6650813 |
| Sequence 13923 | AF118124 | Genbank | 4235636 |
| Sequence 13924 | AF118649 | Genbank | 6840946 |
| Sequence 13925 | AF119297 | Genbank | 4633508 |
| Sequence 13926 | AF123052 | Genbank | 5106556 |
| Sequence 13927 | AF125530 | Genbank | 6563259 |
| Sequence 13928 | AF126023 | Genbank | 6563275 |
| Sequence 13929 | AF126782 | Genbank | 6318547 |
| Sequence 13930 | AF127577 | Genbank | 7019596 |
| Sequence 13931 | AF128527 | Genbank | 4928043 |
| Sequence 13932 | AF128536 | Genbank | 5305705 |
| Sequence 13933 | AF129756 | Genbank | 4337095 |
| Sequence 13934 | AF131738 | Genbank | 4406548 |
| Sequence 13935 | AF131748 | Genbank | 4406563 |
| Sequence 13936 | AF131758 | Genbank | 4406576 |
| Sequence 13937 | AF131771 | Genbank | 4406595 |
| Sequence 13938 | AF131777 | Genbank | 4406602 |
| Sequence 13939 | AF131781 | Genbank | 4406608 |
| Sequence 13940 | AF131814 | Genbank | 4406648 |
| Sequence 13941 | AF131838 | Genbank | 4406677 |
| Sequence 13942 | AF131856 | Genbank | 4406702 |
| Sequence 13943 | AF132939 | Genbank | 4680648 |
| Sequence 13944 | AF132942 | Genbank | 4680654 |
| Sequence 13945 | AF132973 | Genbank | 4680716 |
| Sequence 13946 | AF132984 | Genbank | 4959567 |
| Sequence 13947 | AF134726 | Genbank | 4529886 |
| Sequence 13948 | AF135162 | Genbank | 7259481 |
| Sequence 13949 | AF135488 | Genbank | 5668619 |
| Sequence 13950 | AF146651 | Genbank | 5020073 |
| Sequence 13951 | AF147330 | Genbank | 4761681 |
| Sequence 13952 | AF147378 | Genbank | 4761729 |
| Sequence 13953 | AF147412 | Genbank | 4761763 |
| Sequence 13954 | AF150089 | Genbank | 5107165 |
| Sequence 13955 | AF151052 | Genbank | 7106825 |
| Sequence 13956 | AF151103 | Genbank | 5758136 |
| Sequence 13957 | AF151109 | Genbank | 6166337 |
| Sequence 13958 | AF151801 | Genbank | 4929554 |
| Sequence 13959 | AF151807 | Genbank | 4929566 |
| Sequence 13960 | AF151832 | Genbank | 4929616 |
| Sequence 13961 | AF151835 | Genbank | 4929622 |
| Sequence 13962 | AF151878 | Genbank | 4929708 |
| Sequence 13963 | AF151903 | Genbank | 4929758 |
| Sequence 13964 | AF151905 | Genbank | 4929762 |
| Sequence 13965 | AF151906 | Genbank | 4929764 |
| Sequence 13966 | AF152363 | Genbank | 5669134 |
| Sequence 13967 | AF153273 | Genbank | 5932030 |
| Sequence 13968 | AF153274 | Genbank | 5932031 |
| Sequence 13969 | AF153608 | Genbank | 5231140 |
| Sequence 13970 | AF156965 | Genbank | 5731112 |
| Sequence 13971 | AF158180 | Genbank | 6959307 |
| Sequence 13972 | AF159295 | Genbank | 5714635 |
| Sequence 13973 | AF161384 | Genbank | 6841181 |
| Sequence 13974 | AF161389 | Genbank | 6841191 |
| Sequence 13975 | AF161428 | Genbank | 6841269 |
| Sequence 13976 | AF161448 | Genbank | 6841309 |
| Sequence 13977 | AF161464 | Genbank | 6841451 |
| Sequence 13978 | AF161479 | Genbank | 6841481 |
| Sequence 13979 | AF161481 | Genbank | 6841485 |
| Sequence 13980 | AF161485 | Genbank | 6841493 |
| Sequence 13981 | AF161500 | Genbank | 6841523 |
| Sequence 13982 | AF161503 | Genbank | 6841529 |
| Sequence 13983 | AF161509 | Genbank | 6841541 |
| Sequence 13984 | AF161512 | Genbank | 6841547 |
| Sequence 13985 | AF161541 | Genbank | 6841349 |
| Sequence 13986 | AF161556 | Genbank | 6841379 |
| Sequence 13987 | AF165926 | Genbank | 5817858 |
| Sequence 13988 | AF168956 | Genbank | 5702387 |
| Sequence 13989 | AF172066 | Genbank | 5733811 |
| Sequence 13990 | AF176315 | Genbank | 5823349 |
| Sequence 13991 | AF181896 | Genbank | 6272683 |
| Sequence 13992 | AF187320 | Genbank | 6164847 |
| Sequence 13993 | AF187554 | Genbank | 6653225 |
| Sequence 13994 | AF188745 | Genbank | 6425043 |
| Sequence 13995 | AF189712 | Genbank | 6760474 |
| Sequence 13996 | AF190465 | Genbank | 6120105 |
| Sequence 13997 | AF191018 | Genbank | 6457339 |
| Sequence 13998 | AF191298 | Genbank | 7656642 |
| Sequence 13999 | AF201077 | Genbank | 6456748 |
| Sequence 14000 | AF203815 | Genbank | 6979641 |
| Sequence 14001 | AF205588 | Genbank | 6531675 |
| Sequence 14002 | AF207550 | Genbank | 6470333 |
| Sequence 14003 | AF216305 | Genbank | 6942010 |
| Sequence 14004 | AF228703 | Genbank | 7158868 |
| Sequence 14005 | AF228704 | Genbank | 7158871 |
| Sequence 14006 | AJ000519 | Genbank | 2739214 |
| Sequence 14007 | AJ001014 | Genbank | 3171909 |
| Sequence 14008 | AJ001866 | Genbank | 2969902 |
| Sequence 14009 | AJ001981 | Genbank | 2546978 |
| Sequence 14010 | AJ002030 | Genbank | 2570006 |
| Sequence 14011 | AJ002308 | Genbank | 2959871 |
| Sequence 14012 | AJ002385 | Genbank | 2597930 |
| Sequence 14013 | AJ006470 | Genbank | 3687321 |
| Sequence 14014 | AJ010069 | Genbank | 3483012 |
| Sequence 14015 | AJ010597 | Genbank | 3559873 |
| Sequence 14016 | AJ012409 | Genbank | 3881975 |
| Sequence 14017 | AJ012499 | Genbank | 5441359 |
| Sequence 14018 | AJ223812 | Genbank | 2894518 |
| Sequence 14019 | AJ224442 | Genbank | 2911586 |
| Sequence 14020 | AJ230595 | Genbank | 4583774 |
| Sequence 14021 | AJ230596 | Genbank | 4583775 |
| Sequence 14022 | AJ230605 | Genbank | 4583784 |
| Sequence 14023 | AJ238243 | Genbank | 4826530 |
| Sequence 14024 | AJ238283 | Genbank | 4704219 |
| Sequence 14025 | AJ238374 | Genbank | 6634418 |
| Sequence 14026 | AJ245719 | Genbank | 6688168 |
| Sequence 14027 | AJ249731 | Genbank | 5921469 |
| Sequence 14028 | AJ250042 | Genbank | 6013005 |
| Sequence 14029 | AJ250915 | Genbank | 6996445 |
| Sequence 14030 | AJ251961 | Genbank | 6634453 |
| Sequence 14031 | AJ271216 | Genbank | 6966966 |
| Sequence 14032 | AK000018 | Genbank | 7019828 |
| Sequence 14033 | AK000023 | Genbank | 7019837 |
| Sequence 14034 | AK000035 | Genbank | 7019855 |
| Sequence 14035 | AK000208 | Genbank | 7020141 |
| Sequence 14036 | AK000291 | Genbank | 7020274 |
| Sequence 14037 | AK000348 | Genbank | 7020373 |
| Sequence 14038 | AK000365 | Genbank | 7020402 |
| Sequence 14039 | AK000395 | Genbank | 7020455 |
| Sequence 14040 | AK000426 | Genbank | 7020505 |
| Sequence 14041 | AK000434 | Genbank | 7020520 |
| Sequence 14042 | AK000453 | Genbank | 7020550 |
| Sequence 14043 | AK000462 | Genbank | 7020566 |
| Sequence 14044 | AK000470 | Genbank | 7020580 |
| Sequence 14045 | AK000507 | Genbank | 7020644 |
| Sequence 14046 | AK000548 | Genbank | 7020717 |
| Sequence 14047 | AK000560 | Genbank | 7020738 |
| Sequence 14048 | AK000571 | Genbank | 7020756 |
| Sequence 14049 | AK000585 | Genbank | 7020779 |
| Sequence 14050 | AK000615 | Genbank | 7020827 |
| Sequence 14051 | AK000620 | Genbank | 7020834 |
| Sequence 14052 | AK000624 | Genbank | 7020839 |
| Sequence 14053 | AK000655 | Genbank | 7020888 |
| Sequence 14054 | AK000667 | Genbank | 7020906 |
| Sequence 14055 | AK000669 | Genbank | 7020909 |
| Sequence 14056 | AK000721 | Genbank | 7020985 |
| Sequence 14057 | AK000725 | Genbank | 7020990 |
| Sequence 14058 | AK000739 | Genbank | 7021014 |
| Sequence 14059 | AK000745 | Genbank | 7021025 |
| Sequence 14060 | AK000752 | Genbank | 7021035 |
| Sequence 14061 | AK000765 | Genbank | 7021058 |
| Sequence 14062 | AK000800 | Genbank | 7021104 |
| Sequence 14063 | AK000818 | Genbank | 7021128 |
| Sequence 14064 | AK000826 | Genbank | 7021139 |
| Sequence 14065 | AK000866 | Genbank | 7021190 |
| Sequence 14066 | AK000891 | Genbank | 7021840 |
| Sequence 14067 | AK000933 | Genbank | 7021908 |
| Sequence 14068 | AK000934 | Genbank | 7021909 |
| Sequence 14069 | AK000959 | Genbank | 7021945 |
| Sequence 14070 | AK000991 | Genbank | 7021994 |
| Sequence 14071 | AK001006 | Genbank | 7022017 |
| Sequence 14072 | AK001093 | Genbank | 7022142 |
| Sequence 14073 | AK001105 | Genbank | 7022160 |
| Sequence 14074 | AK001154 | Genbank | 7022228 |
| Sequence 14075 | AK001237 | Genbank | 7022366 |
| Sequence 14076 | AK001285 | Genbank | 7022444 |
| Sequence 14077 | AK001330 | Genbank | 7022520 |
| Sequence 14078 | AK001387 | Genbank | 7022615 |
| Sequence 14079 | AK001413 | Genbank | 7022655 |
| Sequence 14080 | AK001441 | Genbank | 7022699 |
| Sequence 14081 | AK001451 | Genbank | 7022717 |
| Sequence 14082 | AK001469 | Genbank | 7022747 |
| Sequence 14083 | AK001493 | Genbank | 7022783 |
| Sequence 14084 | AK001536 | Genbank | 7022851 |
| Sequence 14085 | AK001571 | Genbank | 7022906 |
| Sequence 14086 | AK001656 | Genbank | 7023047 |
| Sequence 14087 | AK001690 | Genbank | 7023105 |
| Sequence 14088 | AK001691 | Genbank | 7023106 |
| Sequence 14089 | AK001717 | Genbank | 7023151 |
| Sequence 14090 | AK001718 | Genbank | 7023153 |
| Sequence 14091 | AK001747 | Genbank | 7023203 |
| Sequence 14092 | AK001762 | Genbank | 7023231 |
| Sequence 14093 | AK001855 | Genbank | 7023382 |
| Sequence 14094 | AK001877 | Genbank | 7023416 |
| Sequence 14095 | AK001878 | Genbank | 7023418 |
| Sequence 14096 | AK001961 | Genbank | 7023549 |
| Sequence 14097 | AK002002 | Genbank | 7023620 |
| Sequence 14098 | AK002046 | Genbank | 7023689 |
| Sequence 14099 | AK002102 | Genbank | 7023781 |
| Sequence 14100 | AK002169 | Genbank | 7023884 |
| Sequence 14101 | AK002190 | Genbank | 7023914 |
| Sequence 14102 | AL008582 | Genbank | 5360981 |
| Sequence 14103 | AL008639 | Genbank | 6599068 |
| Sequence 14104 | AL008721 | Genbank | 3171883 |
| Sequence 14105 | AL008725 | Genbank | 2791551 |
| Sequence 14106 | AL009051 | Genbank | 2995195 |
| Sequence 14107 | AL009181 | Genbank | 2853179 |
| Sequence 14108 | AL020993 | Genbank | 3980107 |
| Sequence 14109 | AL020995 | Genbank | 5881341 |
| Sequence 14110 | AL021069 | Genbank | 2853183 |
| Sequence 14111 | AL021368 | Genbank | 3080468 |
| Sequence 14112 | AL021546 | Genbank | 2826890 |
| Sequence 14113 | AL021920 | Genbank | 3264535 |
| Sequence 14114 | AL022165 | Genbank | 3281985 |
| Sequence 14115 | AL022238 | Genbank | 4176442 |
| Sequence 14116 | AL022240 | Genbank | 4826471 |
| Sequence 14117 | AL022302 | Genbank | 5262844 |
| Sequence 14118 | AL022312 | Genbank | 4914501 |
| Sequence 14119 | AL022313 | Genbank | 4200326 |
| Sequence 14120 | AL022316 | Genbank | 4691242 |
| Sequence 14121 | AL022320 | Genbank | 4914513 |
| Sequence 14122 | AL022328 | Genbank | 5263010 |
| Sequence 14123 | AL022394 | Genbank | 9581758 |
| Sequence 14124 | AL022395 | Genbank | 4468287 |
| Sequence 14125 | AL022725 | Genbank | 5679748 |
| Sequence 14126 | AL023284 | Genbank | 3355875 |
| Sequence 14127 | AL023494 | Genbank | 5360992 |
| Sequence 14128 | AL023798 | Genbank | 3550031 |
| Sequence 14129 | AL024493 | Genbank | 3288439 |
| Sequence 14130 | AL030996 | Genbank | 3688349 |
| Sequence 14131 | AL031003 | Genbank | 4007185 |
| Sequence 14132 | AL031259 | Genbank | 3790132 |
| Sequence 14133 | AL031274 | Genbank | 3421083 |
| Sequence 14134 | AL031287 | Genbank | 4914533 |
| Sequence 14135 | AL031295 | Genbank | 4376011 |
| Sequence 14136 | AL031368 | Genbank | 4455593 |
| Sequence 14137 | AL031390 | Genbank | 5002609 |
| Sequence 14138 | AL031428 | Genbank | 5931719 |
| Sequence 14139 | AL031431 | Genbank | 4938290 |
| Sequence 14140 | AL031432 | Genbank | 4375969 |
| Sequence 14141 | AL031433 | Genbank | 4826442 |
| Sequence 14142 | AL031447 | Genbank | 4826431 |
| Sequence 14143 | AL031597 | Genbank | 4775640 |
| Sequence 14144 | AL031662 | Genbank | 9716901 |
| Sequence 14145 | AL031667 | Genbank | 9408727 |
| Sequence 14146 | AL031681 | Genbank | 9408729 |
| Sequence 14147 | AL031710 | Genbank | 9798435 |
| Sequence 14148 | AL031716 | Genbank | 4826474 |
| Sequence 14149 | AL031732 | Genbank | 4581343 |
| Sequence 14150 | AL031781 | Genbank | 4038570 |
| Sequence 14151 | AL032821 | Genbank | 4572584 |
| Sequence 14152 | AL033377 | Genbank | 4826462 |
| Sequence 14153 | AL033397 | Genbank | 4902626 |
| Sequence 14154 | AL033522 | Genbank | 4191258 |
| Sequence 14155 | AL033531 | Genbank | 6807582 |
| Sequence 14156 | AL033543 | Genbank | 4581346 |
| Sequence 14157 | AL034417 | Genbank | 5102616 |
| Sequence 14158 | AL034548 | Genbank | 7263904 |
| Sequence 14159 | AL035209 | Genbank | 4160217 |
| Sequence 14160 | AL035413 | Genbank | 6010110 |
| Sequence 14161 | AL035420 | Genbank | 5918161 |
| Sequence 14162 | AL035425 | Genbank | 6465823 |
| Sequence 14163 | AL035458 | Genbank | 6624641 |
| Sequence 14164 | AL035541 | Genbank | 9581764 |
| Sequence 14165 | AL035652 | Genbank | 9650702 |
| Sequence 14166 | AL035681 | Genbank | 4902689 |
| Sequence 14167 | AL035699 | Genbank | 4826515 |
| Sequence 14168 | AL049249 | Genbank | 4499987 |
| Sequence 14169 | AL049282 | Genbank | 4500041 |
| Sequence 14170 | AL049404 | Genbank | 4500192 |
| Sequence 14171 | AL049557 | Genbank | 5596765 |
| Sequence 14172 | AL049563 | Genbank | 4902753 |
| Sequence 14173 | AL049564 | Genbank | 4902757 |
| Sequence 14174 | AL049589 | Genbank | 5679567 |
| Sequence 14175 | AL049696 | Genbank | 5931893 |
| Sequence 14176 | AL049742 | Genbank | 5419783 |
| Sequence 14177 | AL049761 | Genbank | 5738437 |
| Sequence 14178 | AL049766 | Genbank | 5763746 |
| Sequence 14179 | AL049776 | Genbank | 6681704 |
| Sequence 14180 | AL049779 | Genbank | 8176894 |
| Sequence 14181 | AL049829 | Genbank | 8217859 |
| Sequence 14182 | AL049844 | Genbank | 5924019 |
| Sequence 14183 | AL049869 | Genbank | 8217861 |
| Sequence 14184 | AL049929 | Genbank | 4884174 |
| Sequence 14185 | AL049968 | Genbank | 4884217 |
| Sequence 14186 | AL049987 | Genbank | 4884238 |
| Sequence 14187 | AL050021 | Genbank | 4884264 |
| Sequence 14188 | AL050027 | Genbank | 4884266 |
| Sequence 14189 | AL050035 | Genbank | 4884276 |
| Sequence 14190 | AL050083 | Genbank | 4884317 |
| Sequence 14191 | AL050161 | Genbank | 4884375 |
| Sequence 14192 | AL050170 | Genbank | 4884165 |
| Sequence 14193 | AL050215 | Genbank | 4884454 |
| Sequence 14194 | AL050273 | Genbank | 4886450 |
| Sequence 14195 | AL050275 | Genbank | 4886500 |
| Sequence 14196 | AL050290 | Genbank | 4886512 |
| Sequence 14197 | AL050343 | Genbank | 6137008 |
| Sequence 14198 | AL050363 | Genbank | 4914597 |
| Sequence 14199 | AL050383 | Genbank | 4914585 |
| Sequence 14200 | AL050392 | Genbank | 4914613 |
| Sequence 14201 | AL078591 | Genbank | 6562085 |
| Sequence 14202 | AL078621 | Genbank | 6013067 |
| Sequence 14203 | AL079295 | Genbank | 5102607 |
| Sequence 14204 | AL079304 | Genbank | 8176897 |
| Sequence 14205 | AL079342 | Genbank | 6018784 |
| Sequence 14206 | AL080088 | Genbank | 5262504 |
| Sequence 14207 | AL080089 | Genbank | 5262506 |
| Sequence 14208 | AL080097 | Genbank | 5262519 |
| Sequence 14209 | AL080109 | Genbank | 5262535 |
| Sequence 14210 | AL080135 | Genbank | 5262576 |
| Sequence 14211 | AL080144 | Genbank | 5262592 |
| Sequence 14212 | AL080186 | Genbank | 5262664 |
| Sequence 14213 | AL080241 | Genbank | 5730204 |
| Sequence 14214 | AL080250 | Genbank | 5804917 |
| Sequence 14215 | AL080276 | Genbank | 5763753 |
| Sequence 14216 | AL096773 | Genbank | 5918011 |
| Sequence 14217 | AL096803 | Genbank | 6010162 |
| Sequence 14218 | AL109657 | Genbank | 6136991 |
| Sequence 14219 | AL109684 | Genbank | 5689805 |
| Sequence 14220 | AL109925 | Genbank | 7105935 |
| Sequence 14221 | AL110183 | Genbank | 5817095 |
| Sequence 14222 | AL110185 | Genbank | 5817098 |
| Sequence 14223 | AL110193 | Genbank | 5817109 |
| Sequence 14224 | AL110194 | Genbank | 5817111 |
| Sequence 14225 | AL110202 | Genbank | 5817121 |
| Sequence 14226 | AL110208 | Genbank | 5817127 |
| Sequence 14227 | AL110262 | Genbank | 5817224 |
| Sequence 14228 | AL117334 | Genbank | 7248316 |
| Sequence 14229 | AL117424 | Genbank | 5911858 |
| Sequence 14230 | AL117430 | Genbank | 5911865 |
| Sequence 14231 | AL117442 | Genbank | 5911885 |
| Sequence 14232 | AL117558 | Genbank | 5912097 |
| Sequence 14233 | AL117599 | Genbank | 5912167 |
| Sequence 14234 | AL117621 | Genbank | 5912202 |
| Sequence 14235 | AL117648 | Genbank | 5912240 |
| Sequence 14236 | AL117662 | Genbank | 5912257 |
| Sequence 14237 | AL117666 | Genbank | 5912264 |
| Sequence 14238 | AL121576 | Genbank | 8176910 |
| Sequence 14239 | AL121580 | Genbank | 6456854 |
| Sequence 14240 | AL121603 | Genbank | 6434634 |
| Sequence 14241 | AL121656 | Genbank | 7159617 |
| Sequence 14242 | AL121782 | Genbank | 6456831 |
| Sequence 14243 | AL121790 | Genbank | 8919824 |
| Sequence 14244 | AL121913 | Genbank | 7161781 |
| Sequence 14245 | AL121963 | Genbank | 7161783 |
| Sequence 14246 | AL122003 | Genbank | 6580480 |
| Sequence 14247 | AL122013 | Genbank | 7242500 |
| Sequence 14248 | AL122050 | Genbank | 6093244 |
| Sequence 14249 | AL122066 | Genbank | 6102860 |
| Sequence 14250 | AL122089 | Genbank | 6102899 |
| Sequence 14251 | AL133051 | Genbank | 6453468 |
| Sequence 14252 | AL133060 | Genbank | 6453483 |
| Sequence 14253 | AL133094 | Genbank | 6453548 |
| Sequence 14254 | AL133238 | Genbank | 6624577 |
| Sequence 14255 | AL133241 | Genbank | 7630038 |
| Sequence 14256 | AL133243 | Genbank | 6491713 |
| Sequence 14257 | AL133246 | Genbank | 7594587 |
| Sequence 14258 | AL133370 | Genbank | 8248722 |
| Sequence 14259 | AL133453 | Genbank | 7009594 |
| Sequence 14260 | AL133455 | Genbank | 7019734 |
| Sequence 14261 | AL133555 | Genbank | 6599122 |
| Sequence 14262 | AL135744 | Genbank | 6682291 |
| Sequence 14263 | AL135858 | Genbank | 7159622 |
| Sequence 14264 | AL136296 | Genbank | 8217913 |
| Sequence 14265 | AL136504 | Genbank | 7018294 |
| Sequence 14266 | AL137440 | Genbank | 6808001 |
| Sequence 14267 | AL137450 | Genbank | 6808024 |
| Sequence 14268 | AL137489 | Genbank | 6808110 |
| Sequence 14269 | AL137496 | Genbank | 6808137 |
| Sequence 14270 | AL137553 | Genbank | 6808238 |
| Sequence 14271 | AL137567 | Genbank | 6808276 |
| Sequence 14272 | AL137659 | Genbank | 6807778 |
| Sequence 14273 | AL137673 | Genbank | 6807841 |
| Sequence 14274 | AL137681 | Genbank | 6807931 |
| Sequence 14275 | AL137714 | Genbank | 6808128 |
| Sequence 14276 | AL137750 | Genbank | 6808386 |
| Sequence 14277 | AL157428 | Genbank | 7018458 |
| Sequence 14278 | AL157464 | Genbank | 7018479 |
| Sequence 14279 | AP000008 | Genbank | 3132318 |
| Sequence 14280 | AP000031 | Genbank | 3132341 |
| Sequence 14281 | AP000038 | Genbank | 3132348 |
| Sequence 14282 | AP000351 | Genbank | 6069325 |
| Sequence 14283 | AP000354 | Genbank | 5103017 |
| Sequence 14284 | AP000473 | Genbank | 6942329 |
| Sequence 14285 | AP000525 | Genbank | 5931503 |
| Sequence 14286 | AP000526 | Genbank | 5931504 |
| Sequence 14287 | AP000962 | Genbank | 6942330 |
| Sequence 14288 | AP001055 | Genbank | 6693605 |
| Sequence 14289 | AP001136 | Genbank | 6970360 |
| Sequence 14290 | AP001137 | Genbank | 6970361 |
| Sequence 14291 | AP001172 | Genbank | 6983884 |
| Sequence 14292 | AP001343 | Genbank | 7209831 |
| Sequence 14293 | AP001346 | Genbank | 7209834 |
| Sequence 14294 | D10493 | Genbank | 219932 |
| Sequence 14295 | D12485 | Genbank | 219943 |
| Sequence 14296 | D14082 | Genbank | 468933 |
| Sequence 14297 | D14533 | Genbank | 286028 |
| Sequence 14298 | D14540 | Genbank | 467511 |
| Sequence 14299 | D14658 | Genbank | 285940 |
| Sequence 14300 | D14665 | Genbank | 6630617 |
| Sequence 14301 | D14696 | Genbank | 285962 |
| Sequence 14302 | D14710 | Genbank | 559324 |
| Sequence 14303 | D21089 | Genbank | 475156 |
| Sequence 14304 | D21235 | Genbank | 498145 |
| Sequence 14305 | D21262 | Genbank | 434764 |
| Sequence 14306 | D25215 | Genbank | 517114 |
| Sequence 14307 | D25274 | Genbank | 464185 |
| Sequence 14308 | D26600 | Genbank | 565650 |
| Sequence 14309 | D28468 | Genbank | 460704 |
| Sequence 14310 | D29011 | Genbank | 558525 |
| Sequence 14311 | D29643 | Genbank | 473936 |
| Sequence 14312 | D29805 | Genbank | 474986 |
| Sequence 14313 | D29954 | Genbank | 473940 |
| Sequence 14314 | D29958 | Genbank | 473948 |
| Sequence 14315 | D30648 | Genbank | 506337 |
| Sequence 14316 | D31885 | Genbank | 505097 |
| Sequence 14317 | D32053 | Genbank | 2366751 |
| Sequence 14318 | D38112 | Genbank | 644480 |
| Sequence 14319 | D38524 | Genbank | 633070 |
| Sequence 14320 | D42041 | Genbank | 577294 |
| Sequence 14321 | D42063 | Genbank | 924266 |
| Sequence 14322 | D49489 | Genbank | 1136742 |
| Sequence 14323 | D50420 | Genbank | 2618577 |
| Sequence 14324 | D50525 | Genbank | 1167502 |
| Sequence 14325 | D50916 | Genbank | 1469174 |
| Sequence 14326 | D50929 | Genbank | 1469200 |
| Sequence 14327 | D55673 | Genbank | 870746 |
| Sequence 14328 | D63486 | Genbank | 1469885 |
| Sequence 14329 | D63880 | Genbank | 961451 |
| Sequence 14330 | D64015 | Genbank | 2281005 |
| Sequence 14331 | D79205 | Genbank | 1754620 |
| Sequence 14332 | D79986 | Genbank | 1136389 |
| Sequence 14333 | D80000 | Genbank | 1136415 |
| Sequence 14334 | D83004 | Genbank | 1181557 |
| Sequence 14335 | D84273 | Genbank | 3894217 |
| Sequence 14336 | D86962 | Genbank | 1503997 |
| Sequence 14337 | D86985 | Genbank | 6634002 |
| Sequence 14338 | D87003 | Genbank | 2114242 |
| Sequence 14339 | D87127 | Genbank | 1817551 |
| Sequence 14340 | D87328 | Genbank | 1813423 |
| Sequence 14341 | D87666 | Genbank | 1620016 |
| Sequence 14342 | D87684 | Genbank | 1663703 |
| Sequence 14343 | D90209 | Genbank | 220087 |
| Sequence 14344 | E00882 | Genbank | 2169143 |
| Sequence 14345 | E01094 | Genbank | 2169353 |
| Sequence 14346 | E01533 | Genbank | 2169789 |
| Sequence 14347 | E01932 | Genbank | 2170180 |
| Sequence 14348 | E01954 | Genbank | 2170202 |
| Sequence 14349 | E01979 | Genbank | 2170227 |
| Sequence 14350 | E02164 | Genbank | 2170402 |
| Sequence 14351 | E02628 | Genbank | 2170856 |
| Sequence 14352 | E05647 | Genbank | 2173834 |
| Sequence 14353 | E05957 | Genbank | 2174144 |
| Sequence 14354 | E08663 | Genbank | 2176776 |
| Sequence 14355 | E12274 Genbank | 3251108 | |
| Sequence 14356 | E12457 Genbank | 3251290 | |
| Sequence 14357 | E12651 | Genbank | 3251483 |
| Sequence 14358 | E13330 | Genbank | 3252135 |
| Sequence 14359 | E13405 | Genbank | 3252210 |
| Sequence 14360 | J00139 | Genbank | 182715 |
| Sequence 14361 | J01415 | Genbank | 1944628 |
| Sequence 14362 | J02642 | Genbank | 182862 |
| Sequence 14363 | J02960 | Genbank | 178203 |
| Sequence 14364 | J03248 | Genbank | 183053 |
| Sequence 14365 | J03459 | Genbank | 187172 |
| Sequence 14366 | J03537 | Genbank | 337513 |
| Sequence 14367 | J03592 | Genbank | 339722 |
| Sequence 14368 | J03746 | Genbank | 183655 |
| Sequence 14369 | J03779 | Genbank | 179833 |
| Sequence 14370 | J03934 | Genbank | 189245 |
| Sequence 14371 | J04173 | Genbank | 551173 |
| Sequence 14372 | J04801 | Genbank | 190371 |
| Sequence 14373 | J04991 | Genbank | 189425 |
| Sequence 14374 | J05633 | Genbank | 186504 |
| Sequence 14375 | K00558 | Genbank | 340020 |
| Sequence 14376 | K03432 | Genbank | 337377 |
| Sequence 14377 | L01042 | Genbank | 184097 |
| Sequence 14378 | L01057 | Genbank | 292462 |
| Sequence 14379 | L02320 | Genbank | 307365 |
| Sequence 14380 | L05094 | Genbank | 388033 |
| Sequence 14381 | L05491 | Genbank | 292831 |
| Sequence 14382 | L05628 | Genbank | 1835658 |
| Sequence 14383 | L06498 | Genbank | 292442 |
| Sequence 14384 | L07287 | Genbank | 292434 |
| Sequence 14385 | L07633 | Genbank | 186512 |
| Sequence 14386 | L08048 | Genbank | 184250 |
| Sequence 14387 | L08441 | Genbank | 179295 |
| Sequence 14388 | L09054 | Genbank | 307344 |
| Sequence 14389 | L10284 | Genbank | 186522 |
| Sequence 14390 | L10612 | Genbank | 402701 |
| Sequence 14391 | L11066 | Genbank | 307322 |
| Sequence 14392 | L11370 | Genbank | 387674 |
| Sequence 14393 | L12136 | Genbank | 181536 |
| Sequence 14394 | L12387 | Genbank | 459835 |
| Sequence 14395 | L12711 | Genbank | 388890 |
| Sequence 14396 | L13278 | Genbank | 292414 |
| Sequence 14397 | L14778 | Genbank | 306476 |
| Sequence 14398 | L19185 | Genbank | 440307 |
| Sequence 14399 | L19437 | Genbank | 4995970 |
| Sequence 14400 | L19739 | Genbank | 431318 |
| Sequence 14401 | L19783 | Genbank | 404725 |
| Sequence 14402 | L20046 | Genbank | 306741 |
| Sequence 14403 | L20941 | Genbank | 507251 |
| Sequence 14404 | L22569 | Genbank | 348706 |
| Sequence 14405 | L24444 | Genbank | 495860 |
| Sequence 14406 | L25931 | Genbank | 438638 |
| Sequence 14407 | L28809 | Genbank | 454151 |
| Sequence 14408 | L28997 | Genbank | 607027 |
| Sequence 14409 | L29766 | Genbank | 537525 |
| Sequence 14410 | L31610 | Genbank | 1220360 |
| Sequence 14411 | L37127 | Genbank | 4164098 |
| Sequence 14412 | L38995 | Genbank | 704415 |
| Sequence 14413 | L39320 | Genbank | 1226044 |
| Sequence 14414 | L39326 | Genbank | 1228094 |
| Sequence 14415 | L40396 | Genbank | 887357 |
| Sequence 14416 | L43575 | Genbank | 899064 |
| Sequence 14417 | L43964 | Genbank | 951202 |
| Sequence 14418 | L44140 | Genbank | 1203968 |
| Sequence 14419 | L47162 | Genbank | 1082035 |
| Sequence 14420 | L47647 | Genbank | 1000861 |
| Sequence 14421 | L76159 | Genbank | 1246232 |
| Sequence 14422 | L76416 | Genbank | 5566605 |
| Sequence 14423 | M10036 | Genbank | 339840 |
| Sequence 14424 | M11146 | Genbank | 182504 |
| Sequence 14425 | M11147 | Genbank | 182513 |
| Sequence 14426 | M11353 | Genbank | 184092 |
| Sequence 14427 | M12623 | Genbank | 184233 |
| Sequence 14428 | M15205 | Genbank | 339718 |
| Sequence 14429 | M15796 | Genbank | 181271 |
| Sequence 14430 | M16541 | Genbank | 1280204 |
| Sequence 14431 | M17886 | Genbank | 190233 |
| Sequence 14432 | M18048 | Genbank | 337699 |
| Sequence 14433 | M19997 | Genbank | 181968 |
| Sequence 14434 | M21142 | Genbank | 183402 |
| Sequence 14435 | M21575 | Genbank | 1311702 |
| Sequence 14436 | M21895 | Genbank | 189523 |
| Sequence 14437 | M22760 | Genbank | 695359 |
| Sequence 14438 | M22918 | Genbank | 189019 |
| Sequence 14439 | M22920 | Genbank | 189021 |
| Sequence 14440 | M23410 | Genbank | 762884 |
| Sequence 14441 | M24070 | Genbank | 181485 |
| Sequence 14442 | M24194 | Genbank | 187701 |
| Sequence 14443 | M24486 | Genbank | 190785 |
| Sequence 14444 | M24543 | Genbank | 341200 |
| Sequence 14445 | M24690 | Genbank | 341218 |
| Sequence 14446 | M26481 | Genbank | 619789 |
| Sequence 14447 | M26663 | Genbank | 618463 |
| Sequence 14448 | M29064 | Genbank | 337452 |
| Sequence 14449 | M29960 | Genbank | 339886 |
| Sequence 14450 | M31468 | Genbank | 190876 |
| Sequence 14451 | M31520 | Genbank | 337504 |
| Sequence 14452 | M36341 | Genbank | 178984 |
| Sequence 14453 | M36981 | Genbank | 189239 |
| Sequence 14454 | M37104 | Genbank | 179274 |
| Sequence 14455 | M37583 | Genbank | 184059 |
| Sequence 14456 | M38188 | Genbank | 189378 |
| Sequence 14457 | M55150 | Genbank | 182392 |
| Sequence 14458 | M55409 | Genbank | 189596 |
| Sequence 14459 | M58485 | Genbank | 180154 |
| Sequence 14460 | M60457 | Genbank | 181249 |
| Sequence 14461 | M60484 | Genbank | 190225 |
| Sequence 14462 | M60756 | Genbank | 184085 |
| Sequence 14463 | M60857 | Genbank | 181334 |
| Sequence 14464 | M61764 | Genbank | 183702 |
| Sequence 14465 | M63180 | Genbank | 339679 |
| Sequence 14466 | M73547 | Genbank | 190161 |
| Sequence 14467 | M74775 | Genbank | 187151 |
| Sequence 14468 | M75099 | Genbank | 337369 |
| Sequence 14469 | M76299 | Genbank | 188778 |
| Sequence 14470 | M77232 | Genbank | 307392 |
| Sequence 14471 | M86181 | Genbank | 190566 |
| Sequence 14472 | M87339 | Genbank | 1498255 |
| Sequence 14473 | M88006 | Genbank | 178522 |
| Sequence 14474 | M88468 | Genbank | 307197 |
| Sequence 14475 | M90357 | Genbank | 179577 |
| Sequence 14476 | M93036 | Genbank | 182904 |
| Sequence 14477 | M93651 | Genbank | 338038 |
| Sequence 14478 | M94556 | Genbank | 188855 |
| Sequence 14479 | M96982 | Genbank | 338262 |
| Sequence 14480 | S48220 | Genbank | 257451 |
| Sequence 14481 | S54761 | Genbank | 265221 |
| Sequence 14482 | S72871 | Genbank | 639594 |
| Sequence 14483 | S73498 | Genbank | 688010 |
| Sequence 14484 | S73591 | Genbank | 688296 |
| Sequence 14485 | S74678 | Genbank | 241477 |
| Sequence 14486 | S81752 | Genbank | 1438780 |
| Sequence 14487 | S82597 | Genbank | 1699343 |
| Sequence 14488 | S82616 | Genbank | 1699307 |
| Sequence 14489 | U00238 | Genbank | 404860 |
| Sequence 14490 | U02031 | Genbank | 451329 |
| Sequence 14491 | U03886 | Genbank | 458225 |
| Sequence 14492 | U07231 | Genbank | 517195 |
| Sequence 14493 | U07857 | Genbank | 469048 |
| Sequence 14494 | U09466 | Genbank | 495492 |
| Sequence 14495 | U09813 | Genbank | 1008454 |
| Sequence 14496 | U10248 | Genbank | 984280 |
| Sequence 14497 | U10362 | Genbank | 505651 |
| Sequence 14498 | U12404 | Genbank | 531170 |
| Sequence 14499 | U12465 | Genbank | 562073 |
| Sequence 14500 | U12897 | Genbank | 562075 |
| Sequence 14501 | U13616 | Genbank | 608024 |
| Sequence 14502 | U14193 | Genbank | 555899 |
| Sequence 14503 | U14970 | Genbank | 550020 |
| Sequence 14504 | U14972 | Genbank | 550024 |
| Sequence 14505 | U14973 | Genbank | 550026 |
| Sequence 14506 | U17899 | Genbank | 717053 |
| Sequence 14507 | U18321 | Genbank | 603763 |
| Sequence 14508 | U18671 | Genbank | 1293919 |
| Sequence 14509 | U20285 | Genbank | 644878 |
| Sequence 14510 | U20362 | Genbank | 755485 |
| Sequence 14511 | U25182 | Genbank | 799380 |
| Sequence 14512 | U29171 | Genbank | 881618 |
| Sequence 14513 | U29589 | Genbank | 903978 |
| Sequence 14514 | U30826 | Genbank | 1049079 |
| Sequence 14515 | U34683 | Genbank | 1236349 |
| Sequence 14516 | U39817 | Genbank | 1072121 |
| Sequence 14517 | U41060 | Genbank | 1256000 |
| Sequence 14518 | U41195 | Genbank | 1244694 |
| Sequence 14519 | U41448 | Genbank | 3335023 |
| Sequence 14520 | U41668 | Genbank | 1477481 |
| Sequence 14521 | U44798 | Genbank | 1174216 |
| Sequence 14522 | U46751 | Genbank | 3077821 |
| Sequence 14523 | U49245 | Genbank | 1216503 |
| Sequence 14524 | U51559 | Genbank | 1236709 |
| Sequence 14525 | U52111 | Genbank | 8331754 |
| Sequence 14526 | U52852 | Genbank | 8370402 |
| Sequence 14527 | U54993 | Genbank | 4097314 |
| Sequence 14528 | U56833 | Genbank | 1465750 |
| Sequence 14529 | U57846 | Genbank | 1373418 |
| Sequence 14530 | U57847 | Genbank | 1373420 |
| Sequence 14531 | U59911 | Genbank | 1403712 |
| Sequence 14532 | U66243 | Genbank | 1772645 |
| Sequence 14533 | U66589 | Genbank | 1575566 |
| Sequence 14534 | U67243 | Genbank | 2228516 |
| Sequence 14535 | U68566 | Genbank | 1916621 |
| Sequence 14536 | U68758 | Genbank | 4097815 |
| Sequence 14537 | U72648 | Genbank | 3914602 |
| Sequence 14538 | U73168 | Genbank | 1613899 |
| Sequence 14539 | U73645 | Genbank | 1737200 |
| Sequence 14540 | U73824 | Genbank | 1857236 |
| Sequence 14541 | U75285 | Genbank | 2315862 |
| Sequence 14542 | U76549 | Genbank | 1673574 |
| Sequence 14543 | U76669 | Genbank | 6672211 |
| Sequence 14544 | U77643 | Genbank | 2062390 |
| Sequence 14545 | U80017 | Genbank | 1737211 |
| Sequence 14546 | U80460 | Genbank | 2341006 |
| Sequence 14547 | U85625 | Genbank | 5091494 |
| Sequence 14548 | U87460 | Genbank | 2076881 |
| Sequence 14549 | U90904 | Genbank | 1913882 |
| Sequence 14550 | U90907 | Genbank | 1913886 |
| Sequence 14551 | U91327 | Genbank | 1871209 |
| Sequence 14552 | U91328 | Genbank | 2088550 |
| Sequence 14553 | U94586 | Genbank | 1946691 |
| Sequence 14554 | U95689 | Genbank | 2804669 |
| Sequence 14555 | U96629 | Genbank | 2341008 |
| Sequence 14556 | V00478 | Genbank | 28244 |
| Sequence 14557 | V00662 | Genbank | 13003 |
| Sequence 14558 | X00351 | Genbank | 28251 |
| Sequence 14559 | X02152 | Genbank | 34312 |
| Sequence 14560 | X03083 | Genbank | 34309 |
| Sequence 14561 | X03205 | Genbank | 36162 |
| Sequence 14562 | X05276 | Genbank | 37201 |
| Sequence 14563 | X05332 | Genbank | 35740 |
| Sequence 14564 | X05857 | Genbank | 31988 |
| Sequence 14565 | X06409 | Genbank | 30204 |
| Sequence 14566 | X14608 | Genbank | 296365 |
| Sequence 14567 | X15822 | Genbank | 30146 |
| Sequence 14568 | X15940 | Genbank | 36129 |
| Sequence 14569 | X16135 | Genbank | 32355 |
| Sequence 14570 | X16560 | Genbank | 30154 |
| Sequence 14571 | X17025 | Genbank | 488749 |
| Sequence 14572 | X17620 | Genbank | 35067 |
| Sequence 14573 | X52839 | Genbank | 36125 |
| Sequence 14574 | X52851 | Genbank | 30167 |
| Sequence 14575 | X52882 | Genbank | 311380 |
| Sequence 14576 | X52897 | Genbank | 24484 |
| Sequence 14577 | X52943 | Genbank | 28912 |
| Sequence 14578 | X56932 | Genbank | 23690 |
| Sequence 14579 | X56998 | Genbank | 37564 |
| Sequence 14580 | X58288 | Genbank | 32455 |
| Sequence 14581 | X59434 | Genbank | 432375 |
| Sequence 14582 | X60221 | Genbank | 509290 |
| Sequence 14583 | X61755 | Genbank | 32387 |
| Sequence 14584 | X63432 | Genbank | 28335 |
| Sequence 14585 | X63657 | Genbank | 296185 |
| Sequence 14586 | X64123 | Genbank | 35819 |
| Sequence 14587 | X65923 | Genbank | 31302 |
| Sequence 14588 | X66975 | Genbank | 32353 |
| Sequence 14589 | X67247 | Genbank | 36149 |
| Sequence 14590 | X67698 | Genbank | 37476 |
| Sequence 14591 | X69910 | Genbank | 297407 |
| Sequence 14592 | X70940 | Genbank | 38455 |
| Sequence 14593 | X71428 | Genbank | 393415 |
| Sequence 14594 | X72841 | Genbank | 297903 |
| Sequence 14595 | X74801 | Genbank | 671526 |
| Sequence 14596 | X74979 | Genbank | 400462 |
| Sequence 14597 | X75593 | Genbank | 452319 |
| Sequence 14598 | X75684 | Genbank | 429092 |
| Sequence 14599 | X75861 | Genbank | 456258 |
| Sequence 14600 | X76013 | Genbank | 531595 |
| Sequence 14601 | X78137 | Genbank | 460770 |
| Sequence 14602 | X80200 | Genbank | 951276 |
| Sequence 14603 | X81713 | Genbank | 558213 |
| Sequence 14604 | X81788 | Genbank | 1045058 |
| Sequence 14605 | X81895 | Genbank | 2370158 |
| Sequence 14606 | X81900 | Genbank | 2274973 |
| Sequence 14607 | X84908 | Genbank | 1502344 |
| Sequence 14608 | X85372 | Genbank | 806563 |
| Sequence 14609 | X87241 | Genbank | 1107686 |
| Sequence 14610 | X87344 | Genbank | 1054740 |
| Sequence 14611 | X87949 | Genbank | 1143491 |
| Sequence 14612 | X91249 | Genbank | 1160185 |
| Sequence 14613 | X93036 | Genbank | 1085025 |
| Sequence 14614 | X94910 | Genbank | 3413292 |
| Sequence 14615 | X95592 | Genbank | 1185118 |
| Sequence 14616 | X96586 | Genbank | 1556398 |
| Sequence 14617 | X97544 | Genbank | 1770563 |
| Sequence 14618 | X98356 | Genbank | 1648869 |
| Sequence 14619 | X99585 | Genbank | 1770518 |
| Sequence 14620 | Y00052 | Genbank | 30308 |
| Sequence 14621 | Y00282 | Genbank | 36048 |
| Sequence 14622 | Y00345 | Genbank | 35569 |
| Sequence 14623 | Y00815 | Genbank | 34266 |
| Sequence 14624 | Y08201 | Genbank | 1552548 |
| Sequence 14625 | Y09836 | Genbank | 1743253 |
| Sequence 14626 | Y10345 | Genbank | 2292905 |
| Sequence 14627 | Y15286 | Genbank | 2584788 |
| Sequence 14628 | Z11692 | Genbank | 31107 |
| Sequence 14629 | Z11793 | Genbank | 36425 |
| Sequence 14630 | Z13009 | Genbank | 31072 |
| Sequence 14631 | Z14244 | Genbank | 30150 |
| Sequence 14632 | Z21507 | Genbank | 38521 |
| Sequence 14633 | Z24724 | Genbank | 505034 |
| Sequence 14634 | Z25749 | Genbank | 550116 |
| Sequence 14635 | Z31695 | Genbank | 469143 |
| Sequence 14636 | Z36832 | Genbank | 533946 |
| Sequence 14637 | Z36849 | Genbank | 533963 |
| Sequence 14638 | Z46973 | Genbank | 987947 |
| Sequence 14639 | Z47087 | Genbank | 860989 |
| Sequence 14640 | Z48950 | Genbank | 761715 |
| Sequence 14641 | Z54246 | Genbank | 1000700 |
| Sequence 14642 | Z68164 | Genbank | 1107692 |
| Sequence 14643 | Z68284 | Genbank | 1130698 |
| Sequence 14644 | Z69711 | Genbank | 1204109 |
| Sequence 14645 | Z69890 | Genbank | 1213361 |
| Sequence 14646 | Z73900 | Genbank | 1340069 |
| Sequence 14647 | Z82242 | Genbank | 1841915 |
| Sequence 14648 | Z82975 | Genbank | 1673497 |
| Sequence 14649 | Z84474 | Genbank | 1903188 |
| Sequence 14650 | Z84812 | Genbank | 1845164 |
| Sequence 14651 | Z85986 | Genbank | 4034056 |
| Sequence 14652 | Z92544 | Genbank | 1869775 |
| Sequence 14653 | Z92845 | Genbank | 2462406 |
| Sequence 14654 | Z93041 | Genbank | 1905788 |
| Sequence 14655 | Z93928 | Genbank | 2578051 |
| Sequence 14656 | Z95113 | Genbank | 6572188 |
| Sequence 14657 | Z95126 | Genbank | 2342581 |
| Sequence 14658 | Z95704 | Genbank | 2121307 |
| Sequence 14659 | Z97053 | Genbank | 9650676 |
| Sequence 14660 | Z97054 | Genbank | 2370077 |
| Sequence 14661 | Z97056 | Genbank | 2832593 |
| Sequence 14662 | Z97353 | Genbank | 4455632 |
| Sequence 14663 | Z97652 | Genbank | 6456822 |
| Sequence 14664 | Z98049 | Genbank | 2462401 |
| Sequence 14665 | Z98749 | Genbank | 4775611 |
| Sequence 14666 | Z98884 | Genbank | 5304861 |
| Sequence 14667 | Z98950 | Genbank | 3036783 |
| Sequence 14668 | Z99128 | Genbank | 3135974 |
| Sequence 14669 | Z99916 | Genbank | 4158122 |
| Sequence 14670 | AA004759 | EST | 1448327 |
| Sequence 14671 | AA007319 | EST | 1463464 |
| Sequence 14672 | AA009421 | EST | 1470619 |
| Sequence 14673 | AA009598 | EST | 1470757 |
| Sequence 14674 | AA010071 | EST | 1471099 |
| Sequence 14675 | AA010798 | EST | 1471844 |
| Sequence 14676 | AA011369 | EST | 1472466 |
| Sequence 14677 | AA011539 | EST | 1472565 |
| Sequence 14678 | AA011569 | EST | 1472595 |
| Sequence 14679 | AA013065 | EST | 1474101 |
| Sequence 14680 | AA018720 | EST | 1481994 |
| Sequence 14681 | AA019036 | EST | 1482627 |
| Sequence 14682 | AA019504 | EST | 1482187 |
| Sequence 14683 | AA020851 | EST | 1484622 |
| Sequence 14684 | AA021306 | EST | 1484995 |
| Sequence 14685 | AA024597 | EST | 1489502 |
| Sequence 14686 | AA025057 | EST | 1489962 |
| Sequence 14687 | AA025122 | EST | 1490037 |
| Sequence 14688 | AA026215 | EST | 1492901 |
| Sequence 14689 | AA026316 | EST | 1492253 |
| Sequence 14690 | AA026353 | EST | 1492674 |
| Sequence 14691 | AA026643 | EST | 1492801 |
| Sequence 14692 | AA026758 | EST | 1492556 |
| Sequence 14693 | AA027229 | EST | 1492149 |
| Sequence 14694 | AA029679 | EST | 1497082 |
| Sequence 14695 | AA034414 | EST | 1506241 |
| Sequence 14696 | AA035260 | EST | 1506772 |
| Sequence 14697 | AA035377 | EST | 1507101 |
| Sequence 14698 | AA037143 | EST | 1512251 |
| Sequence 14699 | AA039589 | EST | 1516014 |
| Sequence 14700 | AA039683 | EST | 1516155 |
| Sequence 14701 | AA039734 | EST | 1516236 |
| Sequence 14702 | AA039876 | EST | 1516172 |
| Sequence 14703 | AA039929 | EST | 1516206 |
| Sequence 14704 | AA040404 | EST | 1516765 |
| Sequence 14705 | AA043048 | EST | 1522583 |
| Sequence 14706 | AA043193 | EST | 1521111 |
| Sequence 14707 | AA043606 | EST | 1521472 |
| Sequence 14708 | AA044663 | EST | 1523043 |
| Sequence 14709 | AA044835 | EST | 1523038 |
| Sequence 14710 | AA046542 | EST | 1524439 |
| Sequence 14711 | AA046810 | EST | 1524915 |
| Sequence 14712 | AA046864 | EST | 1524763 |
| Sequence 14713 | AA047046 | EST | 1524944 |
| Sequence 14714 | AA053607 | EST | 1544534 |
| Sequence 14715 | AA053853 | EST | 1544797 |
| Sequence 14716 | AA053926 | EST | 1544870 |
| Sequence 14717 | AA053942 | EST | 1545004 |
| Sequence 14718 | AA054471 | EST | 1545396 |
| Sequence 14719 | AA056107 | EST | 1548444 |
| Sequence 14720 | AA057352 | EST | 1549991 |
| Sequence 14721 | AA057541 | EST | 1550246 |
| Sequence 14722 | AA057559 | EST | 1550199 |
| Sequence 14723 | AA063079 | EST | 1556632 |
| Sequence 14724 | AA063612 | EST | 1557579 |
| Sequence 14725 | AA065328 | EST | 1929208 |
| Sequence 14726 | AA069023 | EST | 1576401 |
| Sequence 14727 | AA069422 | EST | 1576781 |
| Sequence 14728 | AA069655 | EST | 1577014 |
| Sequence 14729 | AA070277 | EST | 1577637 |
| Sequence 14730 | AA070855 | EST | 1578487 |
| Sequence 14731 | AA071269 | EST | 1578757 |
| Sequence 14732 | AA071402 | EST | 1578773 |
| Sequence 14733 | AA071472 | EST | 1578903 |
| Sequence 14734 | AA074221 | EST | 1614090 |
| Sequence 14735 | AA074611 | EST | 1614677 |
| Sequence 14736 | AA074614 | EST | 1614492 |
| Sequence 14737 | AA074664 | EST | 1614608 |
| Sequence 14738 | AA074831 | EST | 1614875 |
| Sequence 14739 | AA075200 | EST | 1615070 |
| Sequence 14740 | AA075981 | EST | 1615850 |
| Sequence 14741 | AA079050 | EST | 1617942 |
| Sequence 14742 | AA081478 | EST | 1623536 |
| Sequence 14743 | AA082545 | EST | 1624602 |
| Sequence 14744 | AA083712 | EST | 1625772 |
| Sequence 14745 | AA084678 | EST | 1626735 |
| Sequence 14746 | AA084868 | EST | 1626988 |
| Sequence 14747 | AA088189 | EST | 1633764 |
| Sequence 14748 | AA090105 | EST | 1636589 |
| Sequence 14749 | AA092310 | EST | 1637531 |
| Sequence 14750 | AA092324 | EST | 1637545 |
| Sequence 14751 | AA098978 | EST | 1644862 |
| Sequence 14752 | AA099015 | EST | 1644895 |
| Sequence 14753 | AA099080 | EST | 1645522 |
| Sequence 14754 | AA099327 | EST | 1646033 |
| Sequence 14755 | AA100639 | EST | 1646940 |
| Sequence 14756 | AA101116 | EST | 1647692 |
| Sequence 14757 | AA101598 | EST | 1648552 |
| Sequence 14758 | AA101663 | EST | 1648744 |
| Sequence 14759 | AA102442 | EST | 1647356 |
| Sequence 14760 | AA102560 | EST | 1647752 |
| Sequence 14761 | AA114042 | EST | 1667919 |
| Sequence 14762 | AA115474 | EST | 1670145 |
| Sequence 14763 | AA126478 | EST | 1686071 |
| Sequence 14764 | AA126762 | EST | 1686262 |
| Sequence 14765 | AA126841 | EST | 1686323 |
| Sequence 14766 | AA127483 | EST | 1686755 |
| Sequence 14767 | AA127550 | EST | 1686822 |
| Sequence 14768 | AA127886 | EST | 1687193 |
| Sequence 14769 | AA129205 | EST | 1688990 |
| Sequence 14770 | AA129465 | EST | 1689336 |
| Sequence 14771 | AA129935 | EST | 1689511 |
| Sequence 14772 | AA131336 | EST | 1692834 |
| Sequence 14773 | AA132089 | EST | 1693579 |
| Sequence 14774 | AA132394 | EST | 1693885 |
| Sequence 14775 | AA132989 | EST | 1694505 |
| Sequence 14776 | AA133031 | EST | 1694539 |
| Sequence 14777 | AA133736 | EST | 1690768 |
| Sequence 14778 | AA133759 | EST | 1690727 |
| Sequence 14779 | AA134263 | EST | 1691637 |
| Sequence 14780 | AA134609 | EST | 1695597 |
| Sequence 14781 | AA135030 | EST | 1696141 |
| Sequence 14782 | AA135126 | EST | 1696227 |
| Sequence 14783 | AA135254 | EST | 1696373 |
| Sequence 14784 | AA136337 | EST | 1697545 |
| Sequence 14785 | AA136723 | EST | 1697933 |
| Sequence 14786 | AA136761 | EST | 1698008 |
| Sequence 14787 | AA137016 | EST | 1698225 |
| Sequence 14788 | AA142942 | EST | 1712320 |
| Sequence 14789 | AA143324 | EST | 1712695 |
| Sequence 14790 | AA143422 | EST | 1712852 |
| Sequence 14791 | AA147059 | EST | 1716433 |
| Sequence 14792 | AA147948 | EST | 1717455 |
| Sequence 14793 | AA148889 | EST | 1718998 |
| Sequence 14794 | AA149404 | EST | 1719920 |
| Sequence 14795 | AA149518 | EST | 1720336 |
| Sequence 14796 | AA149579 | EST | 1720380 |
| Sequence 14797 | AA149801 | EST | 1720899 |
| Sequence 14798 | AA149826 | EST | 1720961 |
| Sequence 14799 | AA150361 | EST | 1721873 |
| Sequence 14800 | AA150481 | EST | 1721995 |
| Sequence 14801 | AA151140 | EST | 1719383 |
| Sequence 14802 | AA151423 | EST | 1719820 |
| Sequence 14803 | AA151795 | EST | 1720490 |
| Sequence 14804 | AA151809 | EST | 1720504 |
| Sequence 14805 | AA151816 | EST | 1720511 |
| Sequence 14806 | AA152179 | EST | 1721231 |
| Sequence 14807 | AA155999 | EST | 1727666 |
| Sequence 14808 | AA156226 | EST | 1727844 |
| Sequence 14809 | AA158153 | EST | 1732990 |
| Sequence 14810 | AA158669 | EST | 1733480 |
| Sequence 14811 | AA158704 | EST | 1733533 |
| Sequence 14812 | AA159176 | EST | 1733969 |
| Sequence 14813 | AA159347 | EST | 1734149 |
| Sequence 14814 | AA160666 | EST | 1736043 |
| Sequence 14815 | AA161467 | EST | 1735906 |
| Sequence 14816 | AA165437 | EST | 1741453 |
| Sequence 14817 | AA166691 | EST | 1745155 |
| Sequence 14818 | AA166843 | EST | 1745024 |
| Sequence 14819 | AA166946 | EST | 1745322 |
| Sequence 14820 | AA167011 | EST | 1745386 |
| Sequence 14821 | AA167501 | EST | 1745886 |
| Sequence 14822 | AA169786 | EST | 1748121 |
| Sequence 14823 | AA171706 | EST | 1750791 |
| Sequence 14824 | AA173421 | EST | 1753550 |
| Sequence 14825 | AA173705 | EST | 1753883 |
| Sequence 14826 | AA173936 | EST | 1754068 |
| Sequence 14827 | AA176244 | EST | 1757375 |
| Sequence 14828 | AA176455 | EST | 1757713 |
| Sequence 14829 | AA178955 | EST | 1760308 |
| Sequence 14830 | AA179220 | EST | 1760572 |
| Sequence 14831 | AA181891 | EST | 1765368 |
| Sequence 14832 | AA182751 | EST | 1766581 |
| Sequence 14833 | AA182940 | EST | 1766108 |
| Sequence 14834 | AA186328 | EST | 1774446 |
| Sequence 14835 | AA186811 | EST | 1775103 |
| Sequence 14836 | AA190408 | EST | 1779239 |
| Sequence 14837 | AA192099 | EST | 1781923 |
| Sequence 14838 | AA192347 | EST | 1781585 |
| Sequence 14839 | AA194517 | EST | 1784213 |
| Sequence 14840 | AA195093 | EST | 1784783 |
| Sequence 14841 | AA203477 | EST | 1799204 |
| Sequence 14842 | AA204856 | EST | 1802881 |
| Sequence 14843 | AA205970 | EST | 1801341 |
| Sequence 14844 | AA206446 | EST | 1801826 |
| Sequence 14845 | AA206761 | EST | 1802148 |
| Sequence 14846 | AA210909 | EST | 1809555 |
| Sequence 14847 | AA214548 | EST | 1813173 |
| Sequence 14848 | AA215640 | EST | 1815385 |
| Sequence 14849 | AA216047 | EST | 1816018 |
| Sequence 14850 | AA216061 | EST | 1816048 |
| Sequence 14851 | AA219137 | EST | 1833202 |
| Sequence 14852 | AA220941 | EST | 1839687 |
| Sequence 14853 | AA223990 | EST | 1844515 |
| Sequence 14854 | AA224985 | EST | 1846276 |
| Sequence 14855 | AA225438 | EST | 1846765 |
| Sequence 14856 | AA225485 | EST | 1846812 |
| Sequence 14857 | AA225653 | EST | 1846970 |
| Sequence 14858 | AA225814 | EST | 1847200 |
| Sequence 14859 | AA226768 | EST | 1848466 |
| Sequence 14860 | AA227285 | EST | 1848839 |
| Sequence 14861 | AA229713 | EST | 1851876 |
| Sequence 14862 | AA232183 | EST | 1855538 |
| Sequence 14863 | AA235835 | EST | 1860273 |
| Sequence 14864 | AA243394 | EST | 1874187 |
| Sequence 14865 | AA243646 | EST | 1874456 |
| Sequence 14866 | AA243774 | EST | 1874594 |
| Sequence 14867 | AA244401 | EST | 1875185 |
| Sequence 14868 | AA247569 | EST | 1879338 |
| Sequence 14869 | AA251128 | EST | 1886090 |
| Sequence 14870 | AA252998 | EST | 1882921 |
| Sequence 14871 | AA257006 | EST | 1891135 |
| Sequence 14872 | AA258030 | EST | 1894480 |
| Sequence 14873 | AA258271 | EST | 1893453 |
| Sequence 14874 | AA258394 | EST | 1893536 |
| Sequence 14875 | AA261894 | EST | 1897728 |
| Sequence 14876 | AA261960 | EST | 1898149 |
| Sequence 14877 | AA262181 | EST | 1898452 |
| Sequence 14878 | AA262425 | EST | 1897921 |
| Sequence 14879 | AA262871 | EST | 1898592 |
| Sequence 14880 | AA263047 | EST | 1898845 |
| Sequence 14881 | AA278243 | EST | 1920183 |
| Sequence 14882 | AA278787 | EST | 1920108 |
| Sequence 14883 | AA279139 | EST | 1920605 |
| Sequence 14884 | AA279421 | EST | 1920886 |
| Sequence 14885 | AA280206 | EST | 1921881 |
| Sequence 14886 | AA280658 | EST | 1923453 |
| Sequence 14887 | AA280848 | EST | 1923546 |
| Sequence 14888 | AA282253 | EST | 1925187 |
| Sequence 14889 | AA285040 | EST | 1927721 |
| Sequence 14890 | AA285126 | EST | 1928107 |
| Sequence 14891 | AA292069 | EST | 1940055 |
| Sequence 14892 | AA292296 | EST | 1940276 |
| Sequence 14893 | AA294863 | EST | 1947208 |
| Sequence 14894 | AA295158 | EST | 1947513 |
| Sequence 14895 | AA296277 | EST | 1948652 |
| Sequence 14896 | AA296690 | EST | 1949182 |
| Sequence 14897 | AA297999 | EST | 1950582 |
| Sequence 14898 | AA300796 | EST | 1953128 |
| Sequence 14899 | AA303856 | EST | 1956421 |
| Sequence 14900 | AA304244 | EST | 1956597 |
| Sequence 14901 | AA304272 | EST | 1956615 |
| Sequence 14902 | AA305667 | EST | 1958242 |
| Sequence 14903 | AA306121 | EST | 1958449 |
| Sequence 14904 | AA306470 | EST | 1958798 |
| Sequence 14905 | AA307154 | EST | 1959482 |
| Sequence 14906 | AA307818 | EST | 1960145 |
| Sequence 14907 | AA308297 | EST | 1960626 |
| Sequence 14908 | AA308596 | EST | 1961163 |
| Sequence 14909 | AA311749 | EST | 1964076 |
| Sequence 14910 | AA313317 | EST | 1965646 |
| Sequence 14911 | AA313802 | EST | 1966131 |
| Sequence 14912 | AA314008 | EST | 1966337 |
| Sequence 14913 | AA314493 | EST | 1966822 |
| Sequence 14914 | AA314869 | EST | 1967218 |
| Sequence 14915 | AA315072 | EST | 1967411 |
| Sequence 14916 | AA315192 | EST | 1967541 |
| Sequence 14917 | AA316110 | EST | 1968439 |
| Sequence 14918 | AA316272 | EST | 1968601 |
| Sequence 14919 | AA316868 | EST | 1969196 |
| Sequence 14920 | AA317537 | EST | 1969886 |
| Sequence 14921 | AA318361 | EST | 1970709 |
| Sequence 14922 | AA319855 | EST | 1972181 |
| Sequence 14923 | AA325081 | EST | 1977579 |
| Sequence 14924 | AA328973 | EST | 1981216 |
| Sequence 14925 | AA329553 | EST | 1982040 |
| Sequence 14926 | AA334936 | EST | 1987254 |
| Sequence 14927 | AA339220 | EST | 1991456 |
| Sequence 14928 | AA341254 | EST | 1993491 |
| Sequence 14929 | AA342547 | EST | 1994804 |
| Sequence 14930 | AA343888 | EST | 1996146 |
| Sequence 14931 | AA348129 | EST | 2000440 |
| Sequence 14932 | AA355326 | EST | 2007708 |
| Sequence 14933 | AA359995 | EST | 2012346 |
| Sequence 14934 | AA362311 | EST | 2014781 |
| Sequence 14935 | AA362547 | EST | 2014916 |
| Sequence 14936 | AA363030 | EST | 2015589 |
| Sequence 14937 | AA363971 | EST | 2016289 |
| Sequence 14938 | AA364506 | EST | 2016845 |
| Sequence 14939 | AA371595 | EST | 2024137 |
| Sequence 14940 | AA371677 | EST | 2024220 |
| Sequence 14941 | AA371861 | EST | 2024387 |
| Sequence 14942 | AA376859 | EST | 2029176 |
| Sequence 14943 | AA377534 | EST | 2029863 |
| Sequence 14944 | AA378981 | EST | 2031371 |
| Sequence 14945 | AA379089 | EST | 2031419 |
| Sequence 14946 | AA382161 | EST | 2034479 |
| Sequence 14947 | AA382953 | EST | 2035271 |
| Sequence 14948 | AA393055 | EST | 2046151 |
| Sequence 14949 | AA393236 | EST | 2046205 |
| Sequence 14950 | AA397442 | EST | 2050483 |
| Sequence 14951 | AA398254 | EST | 2051363 |
| Sequence 14952 | AA400319 | EST | 2054395 |
| Sequence 14953 | AA400551 | EST | 2054422 |
| Sequence 14954 | AA400632 | EST | 2054503 |
| Sequence 14955 | AA401792 | EST | 2057276 |
| Sequence 14956 | AA402613 | EST | 2056402 |
| Sequence 14957 | AA402800 | EST | 2056553 |
| Sequence 14958 | AA404535 | EST | 2059268 |
| Sequence 14959 | AA404961 | EST | 2063336 |
| Sequence 14960 | AA405814 | EST | 2063797 |
| Sequence 14961 | AA406106 | EST | 2064105 |
| Sequence 14962 | AA406543 | EST | 2064657 |
| Sequence 14963 | AA410626 | EST | 2069851 |
| Sequence 14964 | AA411013 | EST | 2070137 |
| Sequence 14965 | AA411736 | EST | 2069397 |
| Sequence 14966 | AA412286 | EST | 2070857 |
| Sequence 14967 | AA412695 | EST | 2071301 |
| Sequence 14968 | AA417129 | EST | 2077272 |
| Sequence 14969 | AA417185 | EST | 2077292 |
| Sequence 14970 | AA418317 | EST | 2080146 |
| Sequence 14971 | AA418533 | EST | 2080314 |
| Sequence 14972 | AA418734 | EST | 2080535 |
| Sequence 14973 | AA419146 | EST | 2078944 |
| Sequence 14974 | AA420564 | EST | 2094461 |
| Sequence 14975 | AA420675 | EST | 2094553 |
| Sequence 14976 | AA420705 | EST | 2094611 |
| Sequence 14977 | AA420873 | EST | 2094306 |
| Sequence 14978 | AA421675 | EST | 2100533 |
| Sequence 14979 | AA422114 | EST | 2100938 |
| Sequence 14980 | AA424037 | EST | 2103034 |
| Sequence 14981 | AA425108 | EST | 2107178 |
| Sequence 14982 | AA425723 | EST | 2106443 |
| Sequence 14983 | AA426125 | EST | 2106597 |
| Sequence 14984 | AA426618 | EST | 2107298 |
| Sequence 14985 | AA427438 | EST | 2111308 |
| Sequence 14986 | AA427994 | EST | 2112068 |
| Sequence 14987 | AA427999 | EST | 2112073 |
| Sequence 14988 | AA428399 | EST | 2110228 |
| Sequence 14989 | AA431300 | EST | 2115008 |
| Sequence 14990 | AA431320 | EST | 2115028 |
| Sequence 14991 | AA431322 | EST | 2115030 |
| Sequence 14992 | AA431581 | EST | 2115289 |
| Sequence 14993 | AA431812 | EST | 2115520 |
| Sequence 14994 | AA432282 | EST | 2114670 |
| Sequence 14995 | AA434140 | EST | 2139054 |
| Sequence 14996 | AA436116 | EST | 2141030 |
| Sequence 14997 | AA436991 | EST | 2141905 |
| Sequence 14998 | AA437166 | EST | 2142080 |
| Sequence 14999 | AA437224 | EST | 2142138 |
| Sequence 15000 | AA442133 | EST | 2154011 |
| Sequence 15001 | AA443297 | EST | 2155972 |
| Sequence 15002 | AA443762 | EST | 2156437 |
| Sequence 15003 | AA446569 | EST | 2159234 |
| Sequence 15004 | AA447974 | EST | 2161644 |
| Sequence 15005 | AA448292 | EST | 2161962 |
| Sequence 15006 | AA451954 | EST | 2165623 |
| Sequence 15007 | AA453183 | EST | 2166852 |
| Sequence 15008 | AA456223 | EST | 2179433 |
| Sequence 15009 | AA456863 | EST | 2179583 |
| Sequence 15010 | AA458476 | EST | 2183383 |
| Sequence 15011 | AA459991 | EST | 2184875 |
| Sequence 15012 | AA461239 | EST | 2186359 |
| Sequence 15013 | AA461254 | EST | 2186374 |
| Sequence 15014 | AA463447 | EST | 2188331 |
| Sequence 15015 | AA463899 | EST | 2188783 |
| Sequence 15016 | AA464099 | EST | 2188983 |
| Sequence 15017 | AA464353 | EST | 2189237 |
| Sequence 15018 | AA465191 | EST | 2191358 |
| Sequence 15019 | AA465537 | EST | 2191704 |
| Sequence 15020 | AA468049 | EST | 2194583 |
| Sequence 15021 | AA468360 | EST | 2194894 |
| Sequence 15022 | AA470144 | EST | 2197453 |
| Sequence 15023 | AA477119 | EST | 2205803 |
| Sequence 15024 | AA478414 | EST | 2207048 |
| Sequence 15025 | AA478637 | EST | 2207271 |
| Sequence 15026 | AA479488 | EST | 2208044 |
| Sequence 15027 | AA479863 | EST | 2205749 |
| Sequence 15028 | AA479892 | EST | 2204374 |
| Sequence 15029 | AA481045 | EST | 2210597 |
| Sequence 15030 | AA483162 | EST | 2211975 |
| Sequence 15031 | AA483450 | EST | 2212263 |
| Sequence 15032 | AA484155 | EST | 2212968 |
| Sequence 15033 | AA487382 | EST | 2217546 |
| Sequence 15034 | AA487628 | EST | 2217792 |
| Sequence 15035 | AA487671 | EST | 2217835 |
| Sequence 15036 | AA488200 | EST | 2215631 |
| Sequence 15037 | AA488782 | EST | 2218384 |
| Sequence 15038 | AA492042 | EST | 2221604 |
| Sequence 15039 | AA492143 | EST | 2221705 |
| Sequence 15040 | AA492267 | EST | 2221829 |
| Sequence 15041 | AA492423 | EST | 2221985 |
| Sequence 15042 | AA493364 | EST | 2223205 |
| Sequence 15043 | AA493382 | EST | 2223223 |
| Sequence 15044 | AA493522 | EST | 2223363 |
| Sequence 15045 | AA493624 | EST | 2223465 |
| Sequence 15046 | AA493807 | EST | 2223648 |
| Sequence 15047 | AA496002 | EST | 2229323 |
| Sequence 15048 | AA502198 | EST | 2237165 |
| Sequence 15049 | AA503141 | EST | 2238108 |
| Sequence 15050 | AA503404 | EST | 2238371 |
| Sequence 15051 | AA503941 | EST | 2238908 |
| Sequence 15052 | AA504095 | EST | 2240255 |
| Sequence 15053 | AA504531 | EST | 2240691 |
| Sequence 15054 | AA504703 | EST | 2240863 |
| Sequence 15055 | AA505440 | EST | 2241577 |
| Sequence 15056 | AA506953 | EST | 2243392 |
| Sequence 15057 | AA508597 | EST | 2246100 |
| Sequence 15058 | AA513357 | EST | 2251769 |
| Sequence 15059 | AA513367 | EST | 2251779 |
| Sequence 15060 | AA514331 | EST | 2253839 |
| Sequence 15061 | AA514474 | EST | 2254074 |
| Sequence 15062 | AA514538 | EST | 2254138 |
| Sequence 15063 | AA515736 | EST | 2255336 |
| Sequence 15064 | AA520996 | EST | 2261539 |
| Sequence 15065 | AA521142 | EST | 2261685 |
| Sequence 15066 | AA521353 | EST | 2261896 |
| Sequence 15067 | AA522584 | EST | 2263296 |
| Sequence 15068 | AA523005 | EST | 2388772 |
| Sequence 15069 | AA524825 | EST | 2265753 |
| Sequence 15070 | AA525374 | EST | 2266302 |
| Sequence 15071 | AA525960 | EST | 2268029 |
| Sequence 15072 | AA526186 | EST | 2268255 |
| Sequence 15073 | AA526243 | EST | 2268312 |
| Sequence 15074 | AA526569 | EST | 2268638 |
| Sequence 15075 | AA527150 | EST | 2269219 |
| Sequence 15076 | AA527943 | EST | 2270012 |
| Sequence 15077 | AA528066 | EST | 2270135 |
| Sequence 15078 | AA531276 | EST | 2273982 |
| Sequence 15079 | AA531295 | EST | 2274001 |
| Sequence 15080 | AA532807 | EST | 2278383 |
| Sequence 15081 | AA533483 | EST | 2277579 |
| Sequence 15082 | AA534137 | EST | 2278153 |
| Sequence 15083 | AA534340 | EST | 2278593 |
| Sequence 15084 | AA535571 | EST | 2279824 |
| Sequence 15085 | AA535832 | EST | 2280085 |
| Sequence 15086 | AA541343 | EST | 2287777 |
| Sequence 15087 | AA541537 | EST | 2287971 |
| Sequence 15088 | AA541666 | EST | 2288100 |
| Sequence 15089 | AA548238 | EST | 2318520 |
| Sequence 15090 | AA551141 | EST | 2321393 |
| Sequence 15091 | AA551358 | EST | 2321610 |
| Sequence 15092 | AA551385 | EST | 2321637 |
| Sequence 15093 | AA551581 | EST | 2321833 |
| Sequence 15094 | AA551709 | EST | 2321961 |
| Sequence 15095 | AA551728 | EST | 2321980 |
| Sequence 15096 | AA551770 | EST | 2322022 |
| Sequence 15097 | AA552169 | EST | 2322421 |
| Sequence 15098 | AA552290 | EST | 2322542 |
| Sequence 15099 | AA554025 | EST | 2324564 |
| Sequence 15100 | AA554718 | EST | 2325257 |
| Sequence 15101 | AA554874 | EST | 2325413 |
| Sequence 15102 | AA555194 | EST | 2325733 |
| Sequence 15103 | AA555326 | EST | 2325865 |
| Sequence 15104 | AA557229 | EST | 2327706 |
| Sequence 15105 | AA557760 | EST | 2328237 |
| Sequence 15106 | AA557768 | EST | 2328245 |
| Sequence 15107 | AA563778 | EST | 2335417 |
| Sequence 15108 | AA566072 | EST | 2337711 |
| Sequence 15109 | AA568232 | EST | 2341286 |
| Sequence 15110 | AA570116 | EST | 2344096 |
| Sequence 15111 | AA570171 | EST | 2344151 |
| Sequence 15112 | AA570649 | EST | 2344629 |
| Sequence 15113 | AA570705 | EST | 2344685 |
| Sequence 15114 | AA573315 | EST | 2347843 |
| Sequence 15115 | AA573354 | EST | 2347882 |
| Sequence 15116 | AA573559 | EST | 2348087 |
| Sequence 15117 | AA575939 | EST | 2350454 |
| Sequence 15118 | AA576506 | EST | 2354006 |
| Sequence 15119 | AA578782 | EST | 2356966 |
| Sequence 15120 | AA579375 | EST | 2357559 |
| Sequence 15121 | AA579617 | EST | 2357801 |
| Sequence 15122 | AA579690 | EST | 2357874 |
| Sequence 15123 | AA579834 | EST | 2355161 |
| Sequence 15124 | AA580489 | EST | 2355816 |
| Sequence 15125 | AA582370 | EST | 2359730 |
| Sequence 15126 | AA586421 | EST | 2398416 |
| Sequence 15127 | AA588541 | EST | 2401716 |
| Sequence 15128 | AA593040 | EST | 2408802 |
| Sequence 15129 | AA595014 | EST | 2410364 |
| Sequence 15130 | AA595339 | EST | 2410689 |
| Sequence 15131 | AA599268 | EST | 2432893 |
| Sequence 15132 | AA599661 | EST | 2433286 |
| Sequence 15133 | AA600340 | EST | 2433965 |
| Sequence 15134 | AA600781 | EST | 2434406 |
| Sequence 15135 | AA602357 | EST | 2436335 |
| Sequence 15136 | AA604839 | EST | 2445703 |
| Sequence 15137 | AA604894 | EST | 2445758 |
| Sequence 15138 | AA608917 | EST | 2457345 |
| Sequence 15139 | AA608990 | EST | 2457418 |
| Sequence 15140 | AA609219 | EST | 2457647 |
| Sequence 15141 | AA613550 | EST | 2464588 |
| Sequence 15142 | AA613637 | EST | 2464675 |
| Sequence 15143 | AA618493 | EST | 2505698 |
| Sequence 15144 | AA620307 | EST | 2524246 |
| Sequence 15145 | AA620465 | EST | 2524404 |
| Sequence 15146 | AA621196 | EST | 2525135 |
| Sequence 15147 | AA622806 | EST | 2526682 |
| Sequence 15148 | AA625155 | EST | 2537540 |
| Sequence 15149 | AA625541 | EST | 2537928 |
| Sequence 15150 | AA626377 | EST | 2538764 |
| Sequence 15151 | AA626635 | EST | 2539022 |
| Sequence 15152 | AA626658 | EST | 2539045 |
| Sequence 15153 | AA628413 | EST | 2540800 |
| Sequence 15154 | AA629923 | EST | 2552534 |
| Sequence 15155 | AA629979 | EST | 2552590 |
| Sequence 15156 | AA630335 | EST | 2552946 |
| Sequence 15157 | AA631646 | EST | 2554257 |
| Sequence 15158 | AA631803 | EST | 2554414 |
| Sequence 15159 | AA631901 | EST | 2554512 |
| Sequence 15160 | AA632126 | EST | 2555540 |
| Sequence 15161 | AA632225 | EST | 2555639 |
| Sequence 15162 | AA632270 | EST | 2555684 |
| Sequence 15163 | AA632610 | EST | 2556024 |
| Sequence 15164 | AA633438 | EST | 2555298 |
| Sequence 15165 | AA633491 | EST | 2555351 |
| Sequence 15166 | AA634087 | EST | 2557301 |
| Sequence 15167 | AA634119 | EST | 2557333 |
| Sequence 15168 | AA635616 | EST | 2559458 |
| Sequence 15169 | AA639362 | EST | 2563141 |
| Sequence 15170 | AA640626 | EST | 2565876 |
| Sequence 15171 | AA641037 | EST | 2566287 |
| Sequence 15172 | AA641660 | EST | 2566878 |
| Sequence 15173 | AA644009 | EST | 2569227 |
| Sequence 15174 | AA644184 | EST | 2569402 |
| Sequence 15175 | AA648402 | EST | 2574831 |
| Sequence 15176 | AA649327 | EST | 2575756 |
| Sequence 15177 | AA649926 | EST | 2577254 |
| Sequence 15178 | AA649975 | EST | 2577303 |
| Sequence 15179 | AA651721 | EST | 2583373 |
| Sequence 15180 | AA652395 | EST | 2584047 |
| Sequence 15181 | AA659388 | EST | 2595542 |
| Sequence 15182 | AA659693 | EST | 2595847 |
| Sequence 15183 | AA661905 | EST | 2615996 |
| Sequence 15184 | AA662530 | EST | 2616621 |
| Sequence 15185 | AA663778 | EST | 2617769 |
| Sequence 15186 | AA665413 | EST | 2620026 |
| Sequence 15187 | AA669980 | EST | 2631479 |
| Sequence 15188 | AA676967 | EST | 2657489 |
| Sequence 15189 | AA677766 | EST | 2658288 |
| Sequence 15190 | AA679533 | EST | 2660055 |
| Sequence 15191 | AA683488 | EST | 2670086 |
| Sequence 15192 | AA687192 | EST | 2674397 |
| Sequence 15193 | AA687631 | EST | 2674537 |
| Sequence 15194 | AA687667 | EST | 2674573 |
| Sequence 15195 | AA689603 | EST | 2690530 |
| Sequence 15196 | AA699712 | EST | 2702675 |
| Sequence 15197 | AA699807 | EST | 2702770 |
| Sequence 15198 | AA702016 | EST | 2705129 |
| Sequence 15199 | AA702170 | EST | 2705283 |
| Sequence 15200 | AA705025 | EST | 2714943 |
| Sequence 15201 | AA707310 | EST | 2717228 |
| Sequence 15202 | AA708327 | EST | 2718245 |
| Sequence 15203 | AA714016 | EST | 2726290 |
| Sequence 15204 | AA714225 | EST | 2726499 |
| Sequence 15205 | AA716100 | EST | 2728374 |
| Sequence 15206 | AA716314 | EST | 2728588 |
| Sequence 15207 | AA720680 | EST | 2736815 |
| Sequence 15208 | AA720790 | EST | 2736925 |
| Sequence 15209 | AA721701 | EST | 2737763 |
| Sequence 15210 | AA723194 | EST | 2740971 |
| Sequence 15211 | AA725472 | EST | 2743179 |
| Sequence 15212 | AA725821 | EST | 2743528 |
| Sequence 15213 | AA728892 | EST | 2750251 |
| Sequence 15214 | AA728961 | EST | 2750320 |
| Sequence 15215 | AA729381 | EST | 2750740 |
| Sequence 15216 | AA731128 | EST | 2752332 |
| Sequence 15217 | AA731392 | EST | 2753548 |
| Sequence 15218 | AA731610 | EST | 2753766 |
| Sequence 15219 | AA733210 | EST | 2775319 |
| Sequence 15220 | AA736880 | EST | 2768114 |
| Sequence 15221 | AA742380 | EST | 2784380 |
| Sequence 15222 | AA742661 | EST | 2782167 |
| Sequence 15223 | AA744728 | EST | 2783492 |
| Sequence 15224 | AA744747 | EST | 2783511 |
| Sequence 15225 | AA744963 | EST | 2783727 |
| Sequence 15226 | AA745648 | EST | 2785634 |
| Sequence 15227 | AA746967 | EST | 2786925 |
| Sequence 15228 | AA747290 | EST | 2787248 |
| Sequence 15229 | AA747733 | EST | 2787691 |
| Sequence 15230 | AA749010 | EST | 2788968 |
| Sequence 15231 | AA749335 | EST | 2789293 |
| Sequence 15232 | AA764788 | EST | 2816026 |
| Sequence 15233 | AA765778 | EST | 2817016 |
| Sequence 15234 | AA768205 | EST | 2819220 |
| Sequence 15235 | AA769138 | EST | 2820376 |
| Sequence 15236 | AA769353 | EST | 2820591 |
| Sequence 15237 | AA769538 | EST | 2820776 |
| Sequence 15238 | AA769986 | EST | 2821224 |
| Sequence 15239 | AA770216 | EST | 2821454 |
| Sequence 15240 | AA770613 | EST | 2821851 |
| Sequence 15241 | AA771700 | EST | 2823483 |
| Sequence 15242 | AA774100 | EST | 2825989 |
| Sequence 15243 | AA775415 | EST | 2834749 |
| Sequence 15244 | AA775624 | EST | 2834958 |
| Sequence 15245 | AA778356 | EST | 2837687 |
| Sequence 15246 | AA778660 | EST | 2837991 |
| Sequence 15247 | AA778925 | EST | 2838256 |
| Sequence 15248 | AA779599 | EST | 2838930 |
| Sequence 15249 | AA780299 | EST | 2839630 |
| Sequence 15250 | AA782060 | EST | 2841391 |
| Sequence 15251 | AA782690 | EST | 2842021 |
| Sequence 15252 | AA805264 | EST | 2874014 |
| Sequence 15253 | AA806729 | EST | 2875479 |
| Sequence 15254 | AA807199 | EST | 2876775 |
| Sequence 15255 | AA807824 | EST | 2877230 |
| Sequence 15256 | AA809127 | EST | 2878533 |
| Sequence 15257 | AA810277 | EST | 2879636 |
| Sequence 15258 | AA812398 | EST | 2882009 |
| Sequence 15259 | AA814392 | EST | 2883988 |
| Sequence 15260 | AA814728 | EST | 2884324 |
| Sequence 15261 | AA814888 | EST | 2884484 |
| Sequence 15262 | AA815422 | EST | 2885018 |
| Sequence 15263 | AA824513 | EST | 2896535 |
| Sequence 15264 | AA825977 | EST | 2899289 |
| Sequence 15265 | AA827758 | EST | 2901317 |
| Sequence 15266 | AA827805 | EST | 2900168 |
| Sequence 15267 | AA827816 | EST | 2900179 |
| Sequence 15268 | AA828002 | EST | 2900365 |
| Sequence 15269 | AA828003 | EST | 2900366 |
| Sequence 15270 | AA828621 | EST | 2901720 |
| Sequence 15271 | AA831926 | EST | 2905025 |
| Sequence 15272 | AA831948 | EST | 2905047 |
| Sequence 15273 | AA832521 | EST | 2898667 |
| Sequence 15274 | AA835059 | EST | 2908787 |
| Sequence 15275 | AA835748 | EST | 2910067 |
| Sequence 15276 | AA835869 | EST | 2910188 |
| Sequence 15277 | AA837128 | EST | 2912327 |
| Sequence 15278 | AA838535 | EST | 2913334 |
| Sequence 15279 | AA843197 | EST | 2929715 |
| Sequence 15280 | AA843523 | EST | 2930041 |
| Sequence 15281 | AA844956 | EST | 2931407 |
| Sequence 15282 | AA846735 | EST | 2932875 |
| Sequence 15283 | AA846756 | EST | 2932896 |
| Sequence 15284 | AA846815 | EST | 2932955 |
| Sequence 15285 | AA853937 | EST | 2941475 |
| Sequence 15286 | AA858162 | EST | 2946464 |
| Sequence 15287 | AA861557 | EST | 2953697 |
| Sequence 15288 | AA863230 | EST | 2955709 |
| Sequence 15289 | AA863232 | EST | 2955711 |
| Sequence 15290 | AA863284 | EST | 2955763 |
| Sequence 15291 | AA864497 | EST | 2958810 |
| Sequence 15292 | AA864573 | EST | 2958886 |
| Sequence 15293 | AA865331 | EST | 2957607 |
| Sequence 15294 | AA865552 | EST | 2957828 |
| Sequence 15295 | AA865871 | EST | 2958147 |
| Sequence 15296 | AA866156 | EST | 2958432 |
| Sequence 15297 | AA868706 | EST | 2964151 |
| Sequence 15298 | AA873173 | EST | 2969295 |
| Sequence 15299 | AA873175 | EST | 2969297 |
| Sequence 15300 | AA877181 | EST | 2986258 |
| Sequence 15301 | AA877762 | EST | 2986727 |
| Sequence 15302 | AA883282 | EST | 2992812 |
| Sequence 15303 | AA885293 | EST | 2994370 |
| Sequence 15304 | AA885297 | EST | 2994374 |
| Sequence 15305 | AA886033 | EST | 3001141 |
| Sequence 15306 | AA887219 | EST | 3002327 |
| Sequence 15307 | AA890345 | EST | 3017224 |
| Sequence 15308 | AA890400 | EST | 3017279 |
| Sequence 15309 | AA890449 | EST | 3017328 |
| Sequence 15310 | AA890476 | EST | 3017355 |
| Sequence 15311 | AA902974 | EST | 3038097 |
| Sequence 15312 | AA905362 | EST | 3040485 |
| Sequence 15313 | AA906350 | EST | 3041473 |
| Sequence 15314 | AA908188 | EST | 3047593 |
| Sequence 15315 | AA913039 | EST | 3052431 |
| Sequence 15316 | AA918610 | EST | 3058500 |
| Sequence 15317 | AA926945 | EST | 3075842 |
| Sequence 15374 | AI073499 | EST | 3400143 |
| Sequence 15375 | AI078597 | EST | 3413005 |
| Sequence 15376 | AI081106 | EST | 3417898 |
| Sequence 15377 | AI081419 | EST | 3418211 |
| Sequence 15378 | AI081478 | EST | 3418270 |
| Sequence 15379 | AI081536 | EST | 3418328 |
| Sequence 15380 | AI084607 | EST | 3423030 |
| Sequence 15381 | AI086937 | EST | 3425360 |
| Sequence 15382 | AI087840 | EST | 3426873 |
| Sequence 15383 | AI089289 | EST | 3428348 |
| Sequence 15384 | AI089629 | EST | 3428688 |
| Sequence 15385 | AI091340 | EST | 3430399 |
| Sequence 15386 | AI091999 | EST | 3427197 |
| Sequence 15387 | AI092232 | EST | 3431226 |
| Sequence 15388 | AI092931 | EST | 3431907 |
| Sequence 15389 | AI093444 | EST | 3432420 |
| Sequence 15390 | AI094174 | EST | 3433150 |
| Sequence 15391 | AI095534 | EST | 3434510 |
| Sequence 15392 | AI095786 | EST | 3434762 |
| Sequence 15393 | AI096685 | EST | 3446179 |
| Sequence 15394 | AI114501 | EST | 6359846 |
| Sequence 15395 | AI114683 | EST | 6360028 |
| Sequence 15396 | AI124954 | EST | 3593468 |
| Sequence 15397 | AI125449 | EST | 3593963 |
| Sequence 15398 | AI125479 | EST | 3593993 |
| Sequence 15399 | AI125915 | EST | 3594429 |
| Sequence 15400 | AI127049 | EST | 3595563 |
| Sequence 15401 | AI127824 | EST | 3596338 |
| Sequence 15402 | AI129324 | EST | 3597838 |
| Sequence 15403 | AI129703 | EST | 3598217 |
| Sequence 15404 | AI129827 | EST | 3598341 |
| Sequence 15405 | AI130744 | EST | 3600760 |
| Sequence 15406 | AI130885 | EST | 3600901 |
| Sequence 15407 | AI132921 | EST | 6360237 |
| Sequence 15408 | AI133690 | EST | 6361006 |
| Sequence 15409 | AI13844l | EST | 3644413 |
| Sequence 15410 | AI139393 | EST | 3645365 |
| Sequence 15411 | AIl41041 | EST | 3648498 |
| Sequence 15412 | AI142953 | EST | 3659312 |
| Sequence 15413 | AI143184 | EST | 3664993 |
| Sequence 15414 | AI143226 | EST | 3665035 |
| Sequence 15415 | AI143638 | EST | 3665447 |
| Sequence 15416 | AI143894 | EST | 3665703 |
| Sequence 15417 | AI144216 | EST | 3666025 |
| Sequence 15418 | AI144385 | EST | 3666194 |
| Sequence 15419 | AI146512 | EST | 3674194 |
| Sequence 15420 | AI150844 | EST | 3679313 |
| Sequence 15421 | AI150878 | EST | 3679347 |
| Sequence 15422 | AI159872 | EST | 3693252 |
| Sequence 15423 | AI159999 | EST | 3693379 |
| Sequence 15424 | AI160331 | EST | 3693711 |
| Sequence 15425 | AI160430 | EST | 3693810 |
| Sequence 15426 | AI161162 | EST | 3694467 |
| Sequence 15427 | AI167145 | EST | 3700315 |
| Sequence 15428 | AI167693 | EST | 3700863 |
| Sequence 15429 | AI168090 | EST | 3701260 |
| Sequence 15430 | AI174394 | EST | 3721247 |
| Sequence 15431 | AI174722 | EST | 6361100 |
| Sequence 15432 | AI174834 | EST | 6361229 |
| Sequence 15433 | AI188128 | EST | 3739337 |
| Sequence 15434 | AI188157 | EST | 3739396 |
| Sequence 15435 | AI188575 | EST | 3739784 |
| Sequence 15436 | AI188768 | EST | 3739977 |
| Sequence 15437 | AI189569 | EST | 3740778 |
| Sequence 15438 | AI191342 | EST | 3742551 |
| Sequence 15439 | AI191933 | EST | 3743142 |
| Sequence 15440 | AI199229 | EST | 3751835 |
| Sequence 15441 | AI201859 | EST | 3754465 |
| Sequence 15442 | AI203123 | EST | 3755729 |
| Sequence 15443 | AI207440 | EST | 6361448 |
| Sequence 15444 | AI207448 | EST | 6361456 |
| Sequence 15445 | AI208443 | EST | 3770385 |
| Sequence 15446 | AI218943 | EST | 3801146 |
| Sequence 15447 | AI222237 | EST | 3804440 |
| Sequence 15448 | AI222424 | EST | 3804627 |
| Sequence 15449 | AI222782 | EST | 3804985 |
| Sequence 15450 | AI222806 | EST | 3805009 |
| Sequence 15451 | AI240909 | EST | 3836306 |
| Sequence 15452 | AI241212 | EST | 3836609 |
| Sequence 15453 | AI241763 | EST | 3837160 |
| Sequence 15454 | AI244187 | EST | 3839584 |
| Sequence 15455 | AI245887 | EST | 3841284 |
| Sequence 15456 | AI246248 | EST | 3841645 |
| Sequence 15457 | AI248623 | EST | 3844020 |
| Sequence 15458 | AI249146 | EST | 3844543 |
| Sequence 15459 | AI249680 | EST | 3846209 |
| Sequence 15460 | AI253055 | EST | 3849584 |
| Sequence 15461 | AI253330 | EST | 3850451 |
| Sequence 15462 | AI253414 | EST | 3850369 |
| Sequence 15463 | AI253609 | EST | 3858537 |
| Sequence 15464 | AI254360 | EST | 3861885 |
| Sequence 15465 | AI261577 | EST | 3869780 |
| Sequence 15466 | AI263373 | EST | 3871576 |
| Sequence 15467 | AI264005 | EST | 3872208 |
| Sequence 15468 | AI264289 | EST | 3872492 |
| Sequence 15469 | AI266408 | EST | 3884566 |
| Sequence 15470 | AI266746 | EST | 3884904 |
| Sequence 15471 | AI267341 | EST | 3886508 |
| Sequence 15472 | AI267471 | EST | 3886638 |
| Sequence 15473 | AI267960 | EST | 3887127 |
| Sequence 15474 | AI268542 | EST | 3887709 |
| Sequence 15475 | AI269537 | EST | 3888704 |
| Sequence 15476 | AI271234 | EST | 3890401 |
| Sequence 15477 | AI271608 | EST | 3890775 |
| Sequence 15478 | AI274628 | EST | 3896896 |
| Sequence 15479 | AI276950 | EST | 3899218 |
| Sequence 15480 | AI278211 | EST | 3900479 |
| Sequence 15481 | AI278317 | EST | 3900585 |
| Sequence 15482 | AI278411 | EST | 3900679 |
| Sequence 15483 | AI278998 | EST | 3917232 |
| Sequence 15484 | AI279500 | EST | 3917734 |
| Sequence 15485 | AI279903 | EST | 3918137 |
| Sequence 15486 | AI281560 | EST | 3919793 |
| Sequence 15487 | AI282246 | EST | 3920479 |
| Sequence 15488 | AI283100 | EST | 3921333 |
| Sequence 15489 | AI284545 | EST | 3922778 |
| Sequence 15490 | AI284565 | EST | 3922798 |
| Sequence 15491 | AI289108 | EST | 3932372 |
| Sequence 15492 | AI289752 | EST | 3931593 |
| Sequence 15493 | AI290104 | EST | 3931770 |
| Sequence 15494 | AI291500 | EST | 3934274 |
| Sequence 15495 | AI291613 | EST | 3934387 |
| Sequence 15496 | AI299384 | EST | 3959038 |
| Sequence 15497 | AI299392 | EST | 3959046 |
| Sequence 15498 | AI300346 | EST | 3959692 |
| Sequence 15499 | AI300555 | EST | 3959901 |
| Sequence 15500 | AI301294 | EST | 3960640 |
| Sequence 15501 | AI301904 | EST | 3961250 |
| Sequence 15502 | AI305197 | EST | 3988838 |
| Sequence 15503 | AI305516 | EST | 3990407 |
| Sequence 15504 | AI307736 | EST | 4001954 |
| Sequence 15505 | AI311614 | EST | 4006485 |
| Sequence 15506 | AI313335 | EST | 4018940 |
| Sequence 15507 | AI332968 | EST | 4069527 |
| Sequence 15508 | AI332994 | EST | 4069553 |
| Sequence 15509 | AI334291 | EST | 4070850 |
| Sequence 15510 | AI335537 | EST | 4072464 |
| Sequence 15511 | AI339426 | EST | 4076353 |
| Sequence 15512 | AI339635 | EST | 4076562 |
| Sequence 15513 | AI339952 | EST | 4076879 |
| Sequence 15514 | AI341076 | EST | 4078003 |
| Sequence 15515 | AI342005 | EST | 4078932 |
| Sequence 15516 | AI342217 | EST | 4079144 |
| Sequence 15517 | AI342486 | EST | 4079413 |
| Sequence 15518 | AI344424 | EST | 4081630 |
| Sequence 15519 | AI345709 | EST | 4082915 |
| Sequence 15520 | AI347461 | EST | 4084667 |
| Sequence 15521 | AI348001 | EST | 4085207 |
| Sequence 15522 | AI348771 | EST | 4085989 |
| Sequence 15523 | AI349772 | EST | 4086978 |
| Sequence 15524 | AI352497 | EST | 4089703 |
| Sequence 15525 | AI354464 | EST | 4094617 |
| Sequence 15526 | AI356534 | EST | 4108155 |
| Sequence 15527 | AI361046 | EST | 4112667 |
| Sequence 15528 | AI361513 | EST | 4113134 |
| Sequence 15529 | AI362439 | EST | 4114060 |
| Sequence 15530 | AI366870 | EST | 4136615 |
| Sequence 15531 | AI367932 | EST | 4137677 |
| Sequence 15532 | AI368934 | EST | 4147687 |
| Sequence 15533 | AI369035 | EST | 4147788 |
| Sequence 15534 | AI371024 | EST | 4149777 |
| Sequence 15535 | AI371230 | EST | 4149983 |
| Sequence 15536 | AI375148 | EST | 4175138 |
| Sequence 15537 | AI375903 | EST | 4175893 |
| Sequence 15538 | AI376130 | EST | 4176120 |
| Sequence 15539 | AI376380 | EST | 4186229 |
| Sequence 15540 | AI376921 | EST | 4186774 |
| Sequence 15541 | AI377037 | EST | 4186890 |
| Sequence 15598 | AI565340 | EST | 4523797 |
| Sequence 15599 | AI566010 | EST | 4524462 |
| Sequence 15600 | AI566802 | EST | 4525254 |
| Sequence 15601 | AI567133 | EST | 4525585 |
| Sequence 15602 | AI567453 | EST | 4525905 |
| Sequence 15603 | AI567945 | EST | 4526397 |
| Sequence 15604 | AI569819 | EST | 4533193 |
| Sequence 15605 | AI569945 | EST | 4533319 |
| Sequence 15606 | AI571329 | EST | 4534703 |
| Sequence 15607 | AI571458 | EST | 4534832 |
| Sequence 15608 | AI581200 | EST | 4565576 |
| Sequence 15609 | AI582529 | EST | 4568426 |
| Sequence 15610 | AI582700 | EST | 4568597 |
| Sequence 15611 | AI597662 | EST | 4606710 |
| Sequence 15612 | AI610362 | EST | 4619529 |
| Sequence 15613 | AI619754 | EST | 4628880 |
| Sequence 15614 | AI620259 | EST | 4629385 |
| Sequence 15615 | AI621173 | EST | 4630299 |
| Sequence 15616 | AI624454 | EST | 4649385 |
| Sequence 15617 | AI628617 | EST | 4665417 |
| Sequence 15618 | AI632017 | EST | 4683347 |
| Sequence 15619 | AI632109 | EST | 4683439 |
| Sequence 15620 | AI633086 | EST | 4684416 |
| Sequence 15621 | AI633612 | EST | 4684942 |
| Sequence 15622 | AI634293 | EST | 4685623 |
| Sequence 15623 | AI635449 | EST | 4686779 |
| Sequence 15624 | AI636182 | EST | 4687512 |
| Sequence 15625 | AI636818 | EST | 4688148 |
| Sequence 15626 | AI638334 | EST | 4690568 |
| Sequence 15627 | AI650736 | EST | 4734715 |
| Sequence 15628 | AI651228 | EST | 4735207 |
| Sequence 15629 | AI651423 | EST | 4735402 |
| Sequence 15630 | AI651529 | EST | 4735508 |
| Sequence 15631 | AI652336 | EST | 4736315 |
| Sequence 15632 | AI653693 | EST | 4737672 |
| Sequence 15633 | AI653819 | EST | 4737798 |
| Sequence 15634 | AI654736 | EST | 4738715 |
| Sequence 15635 | AI655590 | EST | 4739569 |
| Sequence 15636 | AI655840 | EST | 4739819 |
| Sequence 15637 | AI659386 | EST | 4762956 |
| Sequence 15638 | AI660901 | EST | 4764484 |
| Sequence 15639 | AI671089 | EST | 4850820 |
| Sequence 15640 | AI673189 | EST | 4852920 |
| Sequence 15641 | AI674314 | EST | 4874794 |
| Sequence 15642 | AI674456 | EST | 4874936 |
| Sequence 15643 | AI674918 | EST | 4875398 |
| Sequence 15644 | AI677810 | EST | 4887992 |
| Sequence 15645 | AI677876 | EST | 4888058 |
| Sequence 15646 | AI679799 | EST | 4889981 |
| Sequence 15647 | AI680555 | EST | 4890737 |
| Sequence 15648 | AI680742 | EST | 4890924 |
| Sequence 15649 | AI681316 | EST | 4891498 |
| Sequence 15650 | AI683072 | EST | 4893254 |
| Sequence 15651 | AI683117 | EST | 4893299 |
| Sequence 15652 | AI683727 | EST | 4893909 |
| Sequence 15653 | AI689427 | EST | 4900721 |
| Sequence 15710 | AI961563 | EST | 5754276 |
| Sequence 15711 | AI971397 | EST | 5768223 |
| Sequence 15712 | AI986177 | EST | 5813454 |
| Sequence 15713 | AL036349 | EST | 5405930 |
| Sequence 15714 | AL037069 | EST | 5928030 |
| Sequence 15715 | AL037845 | EST | 5407176 |
| Sequence 15716 | AL037978 | EST | 5407290 |
| Sequence 15717 | AL038564 | EST | 5407750 |
| Sequence 15718 | AL039253 | EST | 5408327 |
| Sequence 15719 | AL040602 | EST | 5409549 |
| Sequence 15720 | AL042384 | EST | 5421723 |
| Sequence 15721 | AL046811 | EST | 5434873 |
| Sequence 15722 | AL049135 | EST | 4728445 |
| Sequence 15723 | AL118571 | EST | 5924470 |
| Sequence 15724 | AL120354 | EST | 5926253 |
| Sequence 15725 | AL133982 | EST | 6602169 |
| Sequence 15726 | AL134985 | EST | 6603172 |
| Sequence 15727 | AL138451 | EST | 6855132 |
| Sequence 15728 | AL157600 | EST | 7058001 |
| Sequence 15729 | AW008029 | EST | 5856807 |
| Sequence 15730 | AW008673 | EST | 5857451 |
| Sequence 15731 | AW009152 | EST | 5857930 |
| Sequence 15732 | AW016461 | EST | 5865218 |
| Sequence 15733 | AW020561 | EST | 5874091 |
| Sequence 15734 | AW020914 | EST | 5874444 |
| Sequence 15735 | AW021803 | EST | 5875333 |
| Sequence 15736 | AW022479 | EST | 5876009 |
| Sequence 15737 | AW023580 | EST | 5877110 |
| Sequence 15738 | AW026439 | EST | 5879969 |
| Sequence 15739 | AW026700 | EST | 5880153 |
| Sequence 15740 | AW027717 | EST | 5886473 |
| Sequence 15741 | AW029611 | EST | 5888367 |
| Sequence 15742 | AW051380 | EST | 5913650 |
| Sequence 15743 | AW055331 | EST | 5921034 |
| Sequence 15744 | AW057525 | EST | 5933164 |
| Sequence 15745 | AW062700 | EST | 6014085 |
| Sequence 15746 | AW069166 | EST | 6024252 |
| Sequence 15747 | AW075351 | EST | 6030349 |
| Sequence 15748 | AW079672 | EST | 6034824 |
| Sequence 15749 | AW081110 | EST | 6036262 |
| Sequence 15750 | AW081275 | EST | 6036427 |
| Sequence 15751 | AW083877 | EST | 6039029 |
| Sequence 15752 | AW084353 | EST | 6039505 |
| Sequence 15753 | AW084441 | EST | 6039593 |
| Sequence 15754 | AW085581 | EST | 6040733 |
| Sequence 15755 | AW088099 | EST | 6043904 |
| Sequence 15756 | AW089689 | EST | 6047033 |
| Sequence 15757 | AW102924 | EST | 6073539 |
| Sequence 15758 | AW131444 | EST | 6133051 |
| Sequence 15759 | AW151003 | EST | 6198901 |
| Sequence 15760 | AW151170 | EST | 6199068 |
| Sequence 15761 | AW161328 | EST | 6300361 |
| Sequence 15762 | AW161668 | EST | 6300701 |
| Sequence 15763 | AW166819 | EST | 6398344 |
| Sequence 15764 | AW167129 | EST | 6398654 |
| Sequence 15765 | AW168050 | EST | 6399575 |
| Sequence 15766 | AW177001 | EST | 6443038 |
| Sequence 15767 | AW183074 | EST | 6451536 |
| Sequence 15768 | AW188081 | EST | 6462517 |
| Sequence 15769 | AW188641 | EST | 6463001 |
| Sequence 15770 | AW188668 | EST | 6463028 |
| Sequence 15771 | AW188775 | EST | 6463211 |
| Sequence 15772 | AW188814 | EST | 6463250 |
| Sequence 15773 | AW190568 | EST | 6465048 |
| Sequence 15774 | AW235964 | EST | 6568353 |
| Sequence 15775 | AW243325 | EST | 6577165 |
| Sequence 15776 | AW243940 | EST | 6577780 |
| Sequence 15777 | AW271299 | EST | 6658329 |
| Sequence 15778 | AW271852 | EST | 6658882 |
| Sequence 15779 | AW273750 | EST | 6660780 |
| Sequence 15780 | AW290969 | EST | 6697605 |
| Sequence 15781 | AW293305 | EST | 6699867 |
| Sequence 15782 | AW293616 | EST | 6700252 |
| Sequence 15783 | AW294158 | EST | 6700794 |
| Sequence 15784 | AW297885 | EST | 6704510 |
| Sequence 15785 | AW328141 | EST | 6798637 |
| Sequence 15786 | AW360820 | EST | 6865470 |
| Sequence 15787 | AW361122 | EST | 6865772 |
| Sequence 15788 | AW365134 | EST | 6869784 |
| Sequence 15789 | AW368376 | EST | 6873026 |
| Sequence 15790 | AW369111 | EST | 6873761 |
| Sequence 15791 | AW378964 | EST | 6883623 |
| Sequence 15792 | AW381285 | EST | 6885944 |
| Sequence 15793 | AW386920 | EST | 6891579 |
| Sequence 15794 | AW390476 | EST | 6895135 |
| Sequence 15795 | AW394098 | EST | 6898757 |
| Sequence 15796 | AW402596 | EST | 6921298 |
| Sequence 15797 | AW449808 | EST | 6990584 |
| Sequence 15798 | AW449890 | EST | 6990666 |
| Sequence 15799 | AW450105 | EST | 6990881 |
| Sequence 15800 | AW450574 | EST | 6991350 |
| Sequence 15801 | AW450748 | EST | 6991524 |
| Sequence 15802 | AW451232 | EST | 6992008 |
| Sequence 15803 | AW451555 | EST | 6992331 |
| Sequence 15804 | AW451783 | EST | 6992559 |
| Sequence 15805 | AW452088 | EST | 6992864 |
| Sequence 15806 | AW452730 | EST | 6993506 |
| Sequence 15807 | AW468168 | EST | 7038274 |
| Sequence 15808 | AW469281 | EST | 7039387 |
| Sequence 15809 | AW470264 | EST | 7040370 |
| Sequence 15810 | AW473121 | EST | 7043227 |
| Sequence 15811 | AW510526 | EST | N/A |
| Sequence 15812 | AW511443 | EST | N/A |
| Sequence 15813 | AW572897 | EST | 7237630 |
| Sequence 15814 | C00019 | EST | 1432249 |
| Sequence 15815 | C14194 | EST | 1568901 |
| Sequence 15816 | C16523 | EST | 1571230 |
| Sequence 15817 | D53484 | EST | 955381 |
| Sequence 15818 | D61456 | EST | 970232 |
| Sequence 15819 | D61516 | EST | 970472 |
| Sequence 15820 | D81678 | EST | 1179555 |
| Sequence 15821 | F08667 | EST | 672530 |
| Sequence 15822 | F10548 | EST | 683206 |
| Sequence 15823 | F35428 | EST | 4821054 |
| Sequence 15824 | H19093 | EST | 885333 |
| Sequence 15825 | H22094 | EST | 890789 |
| Sequence 15826 | H22238 | EST | 890933 |
| Sequence 15827 | H39906 | EST | 915958 |
| Sequence 15828 | H47418 | EST | 923470 |
| Sequence 15829 | H52272 | EST | 992113 |
| Sequence 15830 | H52374 | EST | 992215 |
| Sequence 15831 | H54240 | EST | 994387 |
| Sequence 15832 | H64134 | EST | 1018935 |
| Sequence 15833 | H67372 | EST | 1026112 |
| Sequence 15834 | H67494 | EST | 1026234 |
| Sequence 15835 | H77381 | EST | 1055470 |
| Sequence 15836 | H78888 | EST | 1056977 |
| Sequence 15837 | H79965 | EST | 1058054 |
| Sequence 15838 | H81340 | EST | 1059429 |
| Sequence 15839 | H90127 | EST | 1080557 |
| Sequence 15840 | N25954 | EST | 1140302 |
| Sequence 15841 | N43012 | EST | 1166756 |
| Sequence 15842 | N48918 | EST | 1190084 |
| Sequence 15843 | N56877 | EST | 1200767 |
| Sequence 15844 | N62671 | EST | 1210500 |
| Sequence 15845 | N66909 | EST | 1219034 |
| Sequence 15846 | N71827 | EST | 1228539 |
| Sequence 15847 | N95674 | EST | 1267942 |
| Sequence 15848 | N95803 | EST | 1268098 |
| Sequence 15849 | R23565 | EST | 778453 |
| Sequence 15850 | R80268 | EST | 856549 |
| Sequence 15851 | R93800 | EST | 967966 |
| Sequence 15852 | R98534 | EST | 985051 |
| Sequence 15853 | T49212 | EST | 651072 |
| Sequence 15854 | T55511 | EST | 657372 |
| Sequence 15855 | T75284 | EST | 692046 |
| Sequence 15856 | T81310 | EST | 704195 |
| Sequence 15857 | T84643 | EST | 712995 |
| Sequence 15858 | T91068 | EST | 722981 |
| Sequence 15859 | U69567 | EST | 2731398 |
| Sequence 15860 | W02490 | EST | 1274488 |
| Sequence 15861 | W04263 | EST | 1276162 |
| Sequence 15862 | W05733 | EST | 1278455 |
| Sequence 15863 | W19899 | EST | 1295768 |
| Sequence 15864 | W21207 | EST | 1298239 |
| Sequence 15865 | W25889 | EST | 1306147 |
| Sequence 15866 | W58768 | EST | 1364280 |
| Sequence 15867 | W60245 | EST | 1367175 |
| Sequence 15868 | W61046 | EST | 1367806 |
| Sequence 15869 | W79674 | EST | 1390063 |
| Sequence 15870 | Z22018 | EST | 288765 |
| Sequence 15871 | Z40994 | EST | 566739 |
| Sequence 15872 | T00880 | NUCPATENT | N/A |
| Sequence 15873 | T19326 | NUCPATENT | N/A |
| Sequence 15874 | T27758 | NUCPATENT | N/A |
| Sequence 15875 | T48099 | NUCPATENT | N/A |
| Sequence 15876 | T92160 | NUCPATENT | N/A |
| Sequence 15877 | T96835 | NUCPATENT | N/A |
| Sequence 15878 | V58586 | NUCPATENT | N/A |
| Sequence 15879 | V59067 | NUCPATENT | N/A |
| Sequence 15880 | V59630 | NUCPATENT | N/A |
| Sequence 15881 | V60015 | NUCPATENT | N/A |
| Sequence 15882 | V65326 | NUCPATENT | N/A |
| Sequence 15883 | V88598 | NUCPATENT | N/A |
| Sequence 15884 | V90354 | NUCPATENT | N/A |
| Sequence 15885 | X02974 | NUCPATENT | N/A |
| Sequence 15886 | X20483 | NUCPATENT | N/A |
| Sequence 15887 | X26850 | NUCPATENT | N/A |
| Sequence 15888 | X27348 | NUCPATENT | N/A |
| Sequence 15889 | X30410 | NUCPATENT | N/A |
| Sequence 15890 | X33813 | NUCPATENT | N/A |
| Sequence 15891 | X84945 | NUCPATENT | N/A |
| Sequence 15892 | X86772 | NUCPATENT | N/A |
| Sequence 15893 | X87396 | NUCPATENT | N/A |
| Sequence 15894 | X87868 | NUCPATENT | N/A |
| Sequence 15895 | X98006 | NUCPATENT | N/A |
| Sequence 15896 | X98010 | NUCPATENT | N/A |
| Sequence 15897 | X98409 | NUCPATENT | N/A |
| Sequence 15898 | X98714 | NUCPATENT | N/A |
| Sequence 15899 | Z14927 | NUCPATENT | N/A |
| Sequence 15900 | Z16190 | NUCPATENT | N/A |
| Sequence 15901 | Z18356 | NUCPATENT | N/A |
| Sequence 15902 | Z24811 | NUCPATENT | N/A |
| Sequence 15903 | Z29718 | NUCPATENT | N/A |
| Sequence 15904 | Z33656 | NUCPATENT | N/A |
| Sequence 15905 | Z34089 | NUCPATENT | N/A |
| Sequence 15906 | Z41384 | NUCPATENT | N/A |
| Sequence 15907 | Z97274 | NUCPATENT | N/A |
| Sequence 15908 | AC28082 | PREPATNUC | N/A |
| Sequence 15909 | AC34864 | PREPATNUC | N/A |
| Sequence 15910 | AC36253 | PREPATNUC | N/A |
| Sequence 15911 | AC38032 | PREPATNUC | N/A |

TABLE 3-6
Sequence 11676: Found in patent publication WO00/05367
AGTACTTCTAGAATTAATTAAGGCGGGCGCCTGCANTCCCAGNTACNCGGNAGGCTGAGG
CAGGAGAATGGCGTGAACCCANGAGGCGGAGCTTGCAGTGAGCTGAGATCGCGCCACTGC
NCTCCAGCCTGGGCAACAGAGTGAGACTCCGTCCCGCTAANAAATNANAAAANAANAANN
CCNGGCCCNCCCNCTGGNGNNTTCCCANATCCCNNTGAATTTTTTTNCCCCCCCC
NNNTGGNCCTGTTTTCTACTAGTGATGATCTGGTAATATACAATTTGTCCAGTAGCCAGT
TTGTTTTTATTGTGTTTTCTAACCATAAGAGATCATTAAAGGCAAAGCCTGTATGACGCT
GTACACACACAAAAAAATGGTCACCGCAGGCCATACTACCAATGAAATGGTAGGTAAACA
AATCTTCTGGTCA
Sequence 11677: Found in patent publication WO99/46375
AGTACTTCTAGAATTAATTAAGGCGGGGACACCACTTTTCAAAGGACTTCTTGGTTTCAG
CATAACCTAAGACAGGGAATTGGGAGCCATCATATGTCACAGTGTTCAGAATTCAAGCAT
ATTTAAGGGCATTTTCTTTGATTCTCAAAGTTCAGCATTCATTTTGAATTGAGAAGCCTA
TACATTTAGCTGACAAAGTGCTTATAGAATTTCTTAACAACTGAACCATTCAAAAGGATT
TTTTTTGTTTAAAACTGGATTTCAATGTAAGCAAATGAAGAAAAAAATATAGATTTCATT
TCCATAGCTTCTTATCCCTGTATTGAGGTAATAAATTGTTTTACTGACAATTTTTCCTTT
TTCTACACTAAAACAATATGTGATATATTTCCCCTCTTGAAGAGGCAATTCATTAAACTC
Sequence 11678: Found in patent publication WO98/54206
AGTACTTCTAGAATTAATTAAAACAGACAATTCTAAAAAAAAAAAAAAAAAAAAAAAAA
AAAAAAAAAAAAAAAAAAAAAAAAAN
Sequence 11679: Found in patent publication WO99/63088
AGTACTTCTAGAATTAATTAAGGCGGGGAGTTTTATCTATTCTTTACATTGGATGCAAAA
TGTATTACTCAAGAAGAGGCATTCGGTATCGAACCATAGATGAACATGATGCCATCATTT
AAGGAAATCCATGGACCAAGGATGGAATACAGATTGATGCTGCCCTATCAATTAATTTTG
GTTTATTAATAGTTTAAAACAATATTCTCTTTTTGAAAATAGTATAAACAGGCCATGCAT
ATAATGTACAGTGTATTACGTAAATATGTAAAGATTCTTCAAGGTAACAAGGGTTTGGGT
TTTGAAATAAACATCTGGATCTTATAGACCGTTCATACAATGGTTTTAGCAAGTTCATAG
TAAGACAAACAAGTCCTATCTTTTTTTTTTTTGGCTGGGGTGGGGGCATTGGTCACATAT
GACCAGTA
Sequence 11680: Found in patent publication WO99/67383
AGTACTTCTAGAATTAATTAAGGCGGGTCCCTGCTTGTCCTATGAACTGCTCAGAGCTCC
TGTCAGTCCAGCTGGGCCTTCTGGGTTCTGGCACCATTTCGTAGCCATTCTCTTTGTATT
TTAAAAGGACGTTATGAAAGGGCTTAGACCAAAATAAATCATAATGTTACTTGAGCCACC
TTATATAGCTGCTTGGAGAGTCTATGTAGTTCTTTCTGCATGCATTAAAAATGTTTAGAA
ATGCTT
Sequence 13322: Found in patent publication WO00/09552
AGTACTTCTAGAATTAATTAAGGCGGGGTCTAGCTCCTTATTTATCTAAATAAAGTTTTA
CTGGAAAAAAAAAAAAAAAAAAAAAAAAAAAAAAAAAAAAAAAAAAAAAAAAAAAAAAAA
AAAAAAAANTTAAAAAAAAAAAAAAAAAAAAAA
Sequence 13323: Found in patent publication WO99/66041
AGTACTTCTAGAATTAATTAAGGCGGGGGAAAGTTAGAGGAACTGAAAGTTTGGGAATAG
GCTGACCACATATTATGCCAGTGACCAGTATGACAGGAGATGGGGCCCTGCTGCCAGTCA
TCTCCACTGAATAAAAAAAAAAAAAAAAAAAAAAAAAAAA
Sequence 13324: Found in patent publication WO99/66041
AGTACTTCTAGAATTAATTAAGGCGGTCGGTTAAAAAAAAAAAAAAAAAAAAAAAAAAAA
AAAAAAAAAAAAAAAAAAAAAAAAAAAAA
Sequence 13325: Found in patent publication WO99/67383
AGTACTTCTAGAATTAATTAAGGCGGGTCCCTGCTTGTCCTATGAACTGCTCAGAGCTCC
TGTCAGTCCAGCTGGGCCTTCTGGGTTCTGGCACCATTTCGTAGCCATTCTCTTTGTATT
TTAAAAGGACGTTATGAAAGGGCTTAGACCAAAATAAATCATAATGTTACTTGAGCCACC
TTATATAGCTGCTTGGAGAGTCTGTGTAGTTCTTTCTGCATGTATTAAAAAGTTTAGAA
A
Sequence 13326: Found in patent publication WO00/06698
GCCCTGGGAAACGATTGTGAACGCNTGAATGAATTGATGACTAANATCCGCTGCGGGGGT
CCTACAGCGCANATGGTAATGCCCGTTCTGACTGGCTGGGAACGGCACCTTAGCAAGATA
CTTAAAAGGCGCCTTCTGTGTGCCACNTGCACTCCACCCTGGGCAACAGAGCAAANACCC
CATCTCAAAAATAAATAAATATATATAAAAAATAAAAAGCTATTTCTAGTTTNATTTCAC
TATAAAGTTTTGCTTTTATTAAA
Sequence 13327: Found in patent publication US6034229
AGTACTTCTAGAATTAATTAAGGCGGGCGGATCATGAGGTCAGGAGTTCGAGACCAGCCT
GGCCAACATGGTGAAACTCCGTGTCTACTAAAAATACAAAAATTGGCTGGGCCGTGGTGG
TTCGCACCTGTAGTCCCAGCTACTGGGGGGGCTGAGGCAGGAGAATGGCTTGAACCCGGG
AGGGTAGAGGTTGCAGTGAGCCAAGATTGTGCCACTGCACTCCAGCCTGGGCGACTGAGC
AAGACTGTCTAAAAAAAAAAAAAAATAAATAAAGNAA
Sequence 13328: Found in patent publication WO00/18925
GCCGTCGTTTTACACCCCGNAANAANCNATANCNATGATNGNTGCCNGTCCCNCTCNNNA
NAGNAATAATAAAANTAATTAANGCGGGTGATTAAATTTCAGTCCATTATGATTTTCCTT
TCTCACATAATTACTTTTTTCTTTTTAGACTTATAAGCTAGCAATTACAGATTTAACTAC
AGCTATCAGCATGGACAAAAATAGTTATACAGCATTTTATAACAGAGCATTATGTTACAC
CAAGATAAGGGAACTTCAAATGGCATTAACAGATTATGGAATTGTGCTGCTTCTTGATGC
TACAGAAACTGTNAAACTAAATACCTTCCTTAATCGTGGACTCATCTACGTAGAACTAGG
CCAGTATGGCTTTGCACTAGAGGATTTTAAAAC
Sequence 15908: Found in patent publication WO98/33916
CTNCGANGNTACTTNCTAGAAATTAATTAAGGGCGGGGNTAGANCAAAAAAAAATAAAAC
AACAACAAAAAAAAACNAAAAATANAAAAAAAAAAAANAANNNANNCAANNNAAAANTAA
AAATTNNNTATTTATTTTAAANTAAAAANTATACTNAACTAATTATTTNAATTAAAGAAA
AAATAANTAAAAAAATTNATAAANAATAAATNTTAAATAAAAAATATT
Sequence 15909: Found in patent publication WO99/14328
AGTACTTCTAGAATTAATTAAGGCGGGGAGAGGAGGCTCTCAATAAATAATCGTGTAACC
TTAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAA
Sequence 15910: Found in patent publication WO99/66041
AGTACTTCTAGAATTAATTAAGGCGGGCAATAAAAGTTGTCATGGTGTGTAAAAAAAAAA
AAAAAAAAAAAAAAAAAAAAAAAAAAAAAAAAAAAAAAAAAAAAAAAAAAAA
Sequence 15911: Found in patent publication WO00/05382
TGGGTGCCGGGCCCCCCCTCGAGAGTACTTCTAGAATTAATTAAGGCGGGAGAAAAGGAA
AAGACATTCCAGACAAAAAGACTAACTTGTCAGAAAGCCCTGTGGCGGAAGGGAGCTTTT
CCAATATGAAGAACTGAGCCTGGAGAGATGGGATGAGGGGGAGTGTCGAACCTTTTAGGC
TTTGTAAAGGAGTTTTGGTTTTCTCCTAATAGCAATGGGATATCTTCCAAGGAATCTCAA
TCAAAAGGGAGAGATGGCTCCGATTGGAATGTCATCCCTGGCTGAAGAGTAGAGGAAGCA
AAAAAAAAAAAAAAAAAAAAANNGNNNNNNCCCNNTNTTTTTTTT

TABLE 4-1

| Sequence ID | Accession |
|---|---|
| Sequence 1 | AA371205 |
| Sequence 2 | AI366380 |
| Sequence 3 | AA315950 |
| Sequence 4 | AA112869 |
| Sequence 5 | AA838003 |
| Sequence 6 | N52554 |
| Sequence 7 | AL039430 |
| Sequence 8 | A1620796 |
| Sequence 9 | A1129714 |
| Sequence 10 | AA885293 |
| Sequence 11 | AA631903 |
| Sequence 12 | AI057093 |
| Sequence 13 | AA680351 |
| Sequence 14 | AA149579 |
| Sequence 15 | H64252 |
| Sequence 16 | AA393816 |
| Sequence 17 | AI924963 |
| Sequence 18 | R18412 |
| Sequence 19 | AI267519 |
| Sequence 20 | AW068579 |
| Sequence 21 | AI051210 |
| Sequence 22 | AA628113 |
| Sequence 23 | AI597980 |
| Sequence 24 | AA195579 |
| Sequence 25 | AA166952 |
| Sequence 26 | AA452288 |
| Sequence 27 | AA188228 |
| Sequence 28 | AA256591 |
| Sequence 29 | R70639 |
| Sequence 30 | AA315689 |
| Sequence 31 | AA133268 |
| Sequence 32 | AA193411 |
| Sequence 33 | AA156244 |
| Sequence 34 | AI298496 |
| Sequence 35 | AI201298 |
| Sequence 36 | W07378 |
| Sequence 37 | AI089431 |
| Sequence 38 | AA649107 |
| Seqdence 39 | AA714567 |
| Sequence 40 | AI744810 |
| Sequence 41 | AA024963 |
| Sequence 42 | R14619 |
| Sequence 43 | AW104561 |
| Sequence 44 | AA296298 |
| Sequence 45 | AI267502 |
| Sequence 46 | AA804659 |
| Sequence 47 | AA053932 |
| Sequence 48 | AI366549 |
| Sequence 49 | AI267256 |
| Sequence 50 | AA453996 |
| Sequence 51 | AA482403 |
| Sequence 52 | AA321222 |
| Sequence 53 | AA482285 |
| Sequence 54 | AA933627 |
| Sequence 55 | AA349223 |
| Sequence 56 | A1267257 |
| Sequence 57 | AA449435 |
| Sequence 58 | AA029881 |
| Sequence 59 | AI267285 |
| Sequence 60 | AA449305 |
| Sequence 61 | AA043133 |
| Sequence 62 | AL120521 |
| Sequence 63 | AA443154 |
| Sequence 64 | AI267185 |
| Sequence 65 | AA431868 |
| Sequence 66 | R28603 |
| Sequence 67 | AI767050 |
| Sequence 68 | AI253345 |
| Sequence 69 | T08429 |
| Sequence 70 | D53496 |
| Sequence 71 | AI267492 |
| Sequence 72 | AA868512 |
| Sequence 73 | T61421 |
| Sequence 74 | AI636911 |
| Sequence 75 | AI674594 |
| Sequence 76 | AA806717 |
| Sequence 77 | AA436125 |
| Sequence 78 | H17871 |
| Sequence 79 | AA075189 |
| Sequence 80 | AA147659 |
| Sequence 81 | AA916753 |
| Sequence 82 | AA173990 |
| Sequence 83 | AI201294 |
| Sequence 84 | AA278445 |
| Sequence 85 | AI536588 |
| Sequence 86 | AA458549 |
| Sequence 87 | AA772075 |
| Sequence 88 | AI499516 |
| Sequence 89 | AA156987 |
| Sequence 90 | AA493512 |
| Sequence 91 | AA522492 |
| Sequence 92 | AA331876 |
| Sequence 93 | R19183 |
| Sequence 94 | AA381480 |
| Sequence 95 | AA625228 |
| Sequence 96 | AI793283 |
| Sequence 97 | AI694211 |
| Sequence 98 | AW023714 |
| Sequence 99 | AA004794 |
| Sequence 100 | AA027037 |
| Sequence 101 | R35797 |
| Sequence 102 | N31529 |
| Sequence 103 | AA578265 |
| Sequence 104 | AI267255 |
| Sequence 105 | AI800250 |
| Sequence 106 | AA551189 |
| Sequence 107 | AA652667 |
| Sequence 108 | AA081218 |
| Sequence 109 | W21047 |
| Sequence 110 | AI216972 |
| Sequence 111 | AA010986 |
| Sequence 112 | N72458 |
| Sequence 113 | AI089923 |
| Sequence 114 | AI039200 |
| Sequence 115 | AL121420 |
| Sequence 116 | AI123347 |
| Sequence 117 | AA654650 |
| Sequence 118 | AI417562 |
| Sequence 119 | AA047587 |
| Sequence 120 | AI954167 |
| Sequence 121 | AA167719 |
| Sequence 122 | AA354341 |
| Sequence 123 | AA304846 |
| Sequence 124 | AA489447 |
| Sequence 125 | AA634799 |
| Sequence 126 | AI619798 |
| Sequence 127 | AI688807 |
| Sequence 128 | AI361119 |
| Sequence 129 | AA286965 |
| Sequence 130 | W37343 |
| Sequence 131 | AI188563 |
| Sequence 132 | W80673 |
| Sequence 133 | AA564067 |
| Sequence 134 | AI033912 |
| Sequence 135 | AI300489 |
| Sequence 136 | AI267454 |
| Sequence 137 | AA555115 |
| Sequence 138 | AA524064 |
| Sequence 139 | AA808211 |
| Sequence 140 | AA248319 |
| Sequence 141 | AA679304 |
| Sequence 142 | AA018249 |
| Sequence 143 | AA303924 |
| Sequence 144 | AA311860 |
| Sequence 145 | AI624709 |
| Sequence 146 | AI219803 |
| Sequence 147 | AA149415 |
| Sequence 148 | AI630228 |
| Sequence 149 | AL047850 |
| Sequence 150 | AA766817 |
| Sequence 151 | AI933533 |
| Sequence 152 | AA603158 |
| Sequence 153 | AA058906 |
| Sequence 154 | AA972598 |
| Sequence 155 | AI760699 |
| Sequence 156 | AA393650 |
| Sequence 157 | AA558281 |
| Sequence 158 | Z17864 |
| Sequence 159 | AL038162 |
| Sequence 160 | AA136944 |
| Sequence 161 | AA493331 |
| Sequence 162 | N32192 |
| Sequence 163 | AA621405 |
| Sequence 164 | AA485102 |
| Sequence 165 | AA262588 |
| Sequence 166 | AA760894 |
| Sequence 167 | AI692513 |
| Sequence 168 | AA312968 |
| Sequence 169 | AA305627 |
| Sequence 170 | AA336621 |
| Sequence 171 | AA521385 |
| Sequence 172 | AA055186 |
| Sequence 173 | R11877 |
| Sequence 174 | AA143021 |
| Sequence 175 | AI797353 |
| Sequence 176 | AA234578 |
| Sequence 177 | D44737 |
| Sequence 178 | AA456721 |
| Sequence 179 | AA310142 |
| Sequence 180 | AA872858 |
| Sequence 181 | AA133742 |
| Sequence 182 | AA028887 |
| Sequence 183 | AW022072 |
| Sequence 184 | AA152276 |
| Sequence 185 | AA443829 |
| Sequence 186 | AI095031 |
| Sequence 187 | AA446789 |
| Sequence 188 | AA307857 |
| Sequence 189 | R10011 |
| Sequence 190 | AI267271 |
| Sequence 191 | AW148750 |
| Sequence 192 | AI692940 |
| Sequence 193 | AA469199 |
| Sequence 194 | AA070335 |
| Sequence 195 | AI873501 |
| Sequence 196 | AI769975 |
| Sequence 197 | AA522549 |
| Sequence 198 | AA142865 |
| Sequence 199 | AA431677 |
| Sequence 200 | AA349232 |
| Sequence 201 | AA521351 |
| Sequence 202 | AI051959 |
| Sequence 203 | AA262123 |
| Sequence 204 | AI266043 |
| Sequence 205 | AA215332 |
| Sequence 206 | AA447189 |
| Sequence 207 | AA037024 |
| Sequence 208 | AW006468 |
| Sequence 209 | AA082477 |
| Sequence 210 | AA094656 |
| Sequence 211 | N40952 |
| Sequence 212 | AA165090 |
| Sequence 213 | AA158274 |
| Sequence 214 | AA196978 |
| Sequence 215 | AI631703 |
| Sequence 216 | AA679434 |
| Sequence 217 | AA306787 |
| Sequence 218 | AA708958 |
| Sequence 219 | AA640474 |
| Sequence 220 | T47718 |
| Sequence 221 | R33762 |
| Sequence 222 | AA857478 |
| Sequence 223 | AI570611 |
| Sequence 224 | AA188312 |
| Sequence 225 | AA765909 |
| Sequence 226 | AA251627 |
| Sequence 227 | AA082063 |
| Sequence 228 | AI557626 |
| Sequence 229 | AA204650 |
| Sequence 230 | AA335273 |
| Sequence 231 | AI346381 |
| Sequence 232 | D82303 |
| Sequence 233 | AI697849 |
| Sequence 234 | AA010869 |
| Sequence 235 | AA833543 |
| Sequence 236 | AA664472 |
| Sequence 237 | AA3I5896 |
| Sequence 238 | N40262 |
| Sequence 239 | AA507505 |
| Sequence 240 | AI183745 |
| Sequence 241 | AI146478 |
| Sequence 242 | AA587806 |
| Sequence 243 | AA355956 |
| Sequence 244 | M225115 |
| Sequence 245 | AA504560 |
| Sequence 246 | AA639999 |
| Sequence 247 | AA420680 |
| Sequence 248 | R63251 |
| Sequence 249 | T67823 |
| Sequence 250 | AA486862 |
| Sequence 251 | R81053 |
| Sequence 252 | AA457365 |
| Sequence 253 | AA640280 |
| Sequence 254 | AA373628 |
| Sequence 255 | AA056744 |
| Sequence 256 | AL120828 |
| Sequence 257 | AI654123 |
| Sequence 258 | AA425414 |
| Sequence 259 | D60110 |
| Sequence 260 | R49052 |
| Sequence 261 | AA878211 |
| Sequence 262 | AI343404 |
| Sequence 263 | AA625538 |
| Sequence 264 | AA134126 |
| Sequence 265 | AI188081 |
| Sequence 266 | AI015245 |
| Sequence 267 | AA460816 |
| Sequence 268 | AI557225 |
| Sequence 269 | AA156790 |
| Sequence 270 | AA397448 |
| Sequence 271 | AA280952 |
| Sequence 272 | AA715365 |
| Sequence 273 | AA652600 |
| Sequence 274 | AA099572 |
| Sequence 275 | AI267384 |
| Sequence 276 | AI884829 |
| Sequence 277 | AA621165 |
| Sequence 278 | AI761728 |
| Sequence 279 | AA122237 |
| Sequence 280 | H51976 |
| Sequence 281 | AI391501 |
| Sequence 282 | AA613772 |
| Sequence 283 | AA578976 |
| Sequence 284 | AA731507 |
| Sequence 285 | AI216358 |
| Sequence 286 | AI557112 |
| Sequence 287 | AL079529 |
| Sequence 288 | AA244003 |
| Sequence 289 | AA625141 |
| Sequence 290 | T59687 |
| Sequence 291 | AA013245 |
| Sequence 292 | AI401220 |
| Sequence 293 | AA324826 |
| Sequence 294 | AL047535 |
| Sequence 295 | AI267522 |
| Sequence 296 | AI682937 |
| Sequence 297 | F08579 |
| Sequence 298 | AI557053 |
| Sequence 299 | R83716 |
| Sequence 300 | AI366467 |
| Sequence 301 | H79449 |
| Sequence 302 | AI267162 |
| Sequence 303 | T06984 |
| Sequence 304 | H83781 |
| Sequence 305 | AAI21145 |
| Sequence 306 | AA314941 |
| Sequence 307 | AI379597 |
| Sequence 308 | AI694477 |
| Sequence 309 | AA210833 |
| Sequence 310 | AA521163 |
| Sequence 311 | AA126834 |
| Sequence 312 | AA437224 |
| Sequence 313 | AA402010 |
| Sequence 314 | AA436197 |
| Sequence 315 | AA313692 |
| Sequence 316 | AA836682 |
| Sequence 317 | AA029276 |
| Sequence 318 | AA017175 |
| Sequence 319 | AA516531 |
| Sequence 320 | AA465494 |
| Sequence 321 | AI394646 |
| Sequence 322 | AA927118 |
| Sequence 323 | AW062373 |
| Sequence 324 | Z20613 |
| Sequence 325 | AA134311 |
| Sequence 326 | AA612864 |
| Sequence 327 | AA631868 |
| Sequence 328 | AI239435 |
| Sequence 329 | T70288 |
| Sequence 330 | AA285043 |
| Sequence 331 | AA705546 |
| Sequence 332 | AW169890 |
| Sequence 333 | AI082617 |
| Sequence 334 | AA345027 |
| Sequence 335 | AI217172 |
| Sequence 336 | M449907 |
| Sequence 337 | AA626511 |
| Sequence 338 | AI002821 |
| Sequence 339 | AA505073 |
| Sequence 340 | T71154 |
| Sequence 341 | AW070326 |
| Sequence 342 | AA366269 |
| Sequence 343 | R71619 |
| Sequence 344 | AA058568 |
| Sequence 345 | AA359418 |
| Sequence 346 | AA305599 |
| Sequence 347 | AI269053 |
| Sequence 348 | AA403121 |
| Sequence 349 | AA514302 |
| Sequence 350 | AA174144 |
| Sequence 351 | AW177204 |
| Sequence 352 | AA580097 |
| Sequence 353 | M664480 |
| Sequence 354 | W60040 |
| Sequence 355 | R71431 |
| Sequence 356 | AA581846 |
| Sequence 357 | AI216968 |
| Sequence 358 | AA243573 |
| Sequence 359 | AL037638 |
| Sequence 360 | R49734 |
| Sequence 361 | AA223433 |
| Sequence 362 | AA157267 |
| Sequence 363 | AL046802 |
| Sequence 364 | AA659719 |
| Sequence 365 | AA333526 |
| Sequence 366 | AA235224 |
| Sequence 367 | AI018034 |
| Sequence 368 | AA284997 |
| Sequence 369 | AA401802 |
| Sequence 370 | T71615 |
| Sequence 371 | R23858 |
| Sequence 372 | AL039698 |
| Sequence 373 | AF017714 |
| Sequence 374 | AI267570 |
| Sequence 375 | AA525386 |
| Sequence 376 | AL037256 |
| Sequence 377 | AI040598 |
| Sequence 378 | W58731 |
| Sequence 379 | AI356805 |
| Sequence 380 | AA033951 |
| Sequence 381 | AA282215 |
| Sequence 382 | AA421795 |
| Sequence 383 | AA767784 |
| Sequence 384 | AA284615 |
| Sequence 385 | R18215 |
| Sequence 386 | AA122080 |
| Sequence 387 | AA157875 |
| Sequence 388 | AA040673 |
| Sequence 389 | M463761 |
| Sequence 390 | AA527805 |
| Sequence 391 | AA036798 |
| Sequence 392 | R25807 |
| Sequence 393 | AA675892 |
| Sequence 394 | W03191 |
| Sequence 395 | AI016031 |
| Sequence 396 | AA258079 |
| Sequence 397 | AA150033 |
| Sequence 398 | AA297647 |
| Sequence 399 | AI610699 |
| Sequence 400 | AA120964 |
| Sequence 401 | AA814091 |
| Sequence 402 | AI474235 |
| Sequence 403 | AA307371 |
| Sequence 404 | M344133 |
| Sequence 405 | AA316649 |
| Sequence 406 | AI694320 |
| Sequence 407 | AA017096 |
| Sequence 408 | M452999 |
| Sequence 409 | M297472 |
| Sequence 410 | W87509 |
| Sequence 411 | M020941 |
| Sequence 412 | R64194 |
| Sequence 413 | AI042293 |
| Sequence 414 | M044118 |
| Sequence 415 | H25806 |
| Sequence 416 | AA383011 |
| Sequence 417 | AA328366 |
| Sequence 418 | AI216969 |
| Sequence 419 | AA165339 |
| Sequence 420 | AA308063 |
| Sequence 421 | N52271 |
| Sequence 422 | AA315718 |
| Sequence 423 | AI554809 |
| Sequence 424 | H81755 |
| Sequence 425 | AA829350 |
| Sequence 426 | R45470 |
| Sequence 427 | AA133652 |
| Sequence 428 | AA405892 |
| Sequence 429 | AW139567 |
| Sequence 430 | AA225114 |
| Sequence 431 | AA343168 |
| Sequence 432 | AA040405 |
| Sequence 433 | AA229745 |
| Sequence 434 | AA830959 |
| Sequence 435 | AW075906 |
| Sequence 436 | AA533227 |
| Sequence 437 | AA079041 |
| Sequence 438 | N31825 |
| Sequence 439 | AA503132 |
| Sequence 440 | AA318969 |
| Sequence 441 | AW020429 |
| Sequence 442 | AA315072 |
| Sequence 443 | AA709208 |
| Sequence 444 | H14446 |
| Sequence 445 | AA548124 |
| Sequence 446 | AI927636 |
| Sequence 447 | AI697249 |
| Sequence 448 | AA146900 |
| Sequence 449 | N94259 |
| Sequence 450 | AA215284 |
| Sequence 451 | AI000877 |
| Sequence 452 | AA373289 |
| Sequence 453 | AA040624 |
| Sequence 454 | AA227789 |
| Sequence 455 | AI378932 |
| Sequence 456 | AA936532 |
| Sequence 457 | AL043113 |
| Sequence 458 | AL121031 |
| Sequence 459 | AA508810 |
| Sequence 460 | AA129003 |
| Sequence 461 | R63823 |
| Sequence 462 | N24785 |
| Sequence 463 | T95139 |
| Sequence 464 | AI480345 |
| Sequence 465 | AA181111 |
| Sequence 466 | AA908961 |
| Sequence 487 | AA836233 |
| Sequence 468 | AA308486 |
| Sequence 469 | AA773998 |
| Sequence 470 | AA644237 |
| Sequence 471 | AA173721 |
| Sequence 472 | AA458528 |
| Sequence 473 | AI400752 |
| Sequence 474 | AA807276 |
| Sequence 475 | AW022792 |
| Sequence 476 | AI989600 |
| Sequence 477 | AI267838 |
| Sequence 478 | AA825265 |
| Sequence 479 | AA229199 |
| Sequence 480 | AW003442 |
| Sequence 481 | AI275711 |
| Sequence 482 | R11581 |
| Sequence 483 | AA931943 |
| Sequence 484 | AA770272 |
| Sequence 485 | D55192 |
| Sequence 486 | AA129551 |
| Sequence 487 | AA772683 |
| Sequence 488 | AA047752 |
| Sequence 489 | AI366471 |
| Sequence 490 | AI267669 |
| Sequence 491 | AA702679 |
| Sequence 492 | AA449544 |
| Sequence 493 | AA133757 |
| Sequence 494 | AA287347 |
| Sequence 495 | AA548129 |
| Sequence 496 | AA355351 |
| Sequence 497 | AA164703 |
| Sequence 498 | H15660 |
| Sequence 499 | AI417910 |
| Sequence 500 | AL041265 |
| Sequence 501 | AI281449 |
| Sequence 502 | AA447021 |
| Sequence 503 | AA032194 |
| Sequence 504 | AI079595 |
| Sequence 505 | AA181179 |
| Sequence 506 | AA368792 |
| Sequence 507 | AA522856 |
| Sequence 508 | AI004135 |
| Sequence 509 | D82197 |
| Sequence 510 | AW083338 |
| Sequence 511 | AI076608 |
| Sequence 512 | AI754013 |
| Sequence 513 | AA088322 |
| Sequence 514 | AI480424 |
| Sequence 515 | AA151796 |
| Sequence 516 | AA034322 |
| Sequence 517 | AI207660 |
| Sequence 518 | T81369 |
| Sequence 519 | AA885902 |
| Sequence 520 | AI332597 |
| Sequence 521 | AA043891 |
| Sequence 522 | AA531606 |
| Sequence 523 | AA504728 |
| Sequence 524 | AA315118 |
| Sequence 525 | AI391717 |
| Sequence 526 | AA337114 |
| Sequence 527 | AA573557 |
| Sequence 528 | AI630584 |
| Sequence 529 | AA534235 |
| Sequence 530 | H56898 |
| Sequence 531 | AI073907 |
| Sequence 532 | AI471561 |
| Sequence 533 | T28974 |
| Sequence 534 | AA307475 |
| Sequence 535 | AI952763 |
| Sequence 536 | AA524893 |
| Sequence 537 | AI927846 |
| Sequence 538 | H12056 |
| Sequence 539 | AA010758 |
| Sequence 540 | AA334403 |
| Sequence 541 | AI625404 |
| Sequence 542 | AI356309 |
| Sequence 543 | AI768908 |
| Sequence 544 | T06925 |
| Sequence 545 | AI656158 |
| Sequence 546 | AI366461 |
| Sequence 547 | T30677 |
| Sequence 548 | AA436472 |
| Sequence 549 | AA628409 |
| Sequence 550 | AA263162 |
| Sequence 551 | AA468167 |
| Sequence 552 | AI929464 |
| Sequence 553 | AA465340 |
| Sequence 554 | AI984636 |
| Sequence 555 | AA687571 |
| Sequence 556 | AA488809 |
| Sequence 557 | AI557235 |
| Sequence 558 | AA609641 |
| Sequence 559 | AI458673 |
| Sequence 560 | AW166338 |
| Sequence 561 | AA984384 |
| Sequence 562 | AI923011 |
| Sequence 563 | AI744818 |
| Sequence 564 | AA708501 |
| Sequence 565 | AA402500 |
| Sequence 566 | AA132633 |
| Sequence 567 | AI523222 |
| Sequence 568 | AA814859 |
| Sequence 569 | H54240 |
| Sequence 570 | AI206223 |
| Sequence 571 | AI627818 |
| Sequence 572 | AI198986 |
| Sequence 573 | AI972279 |
| Sequence 574 | H00112 |
| Sequence 575 | AI267480 |
| Sequence 576 | AI623984 |
| Sequence 577 | AA701667 |
| Sequence 578 | AA988520 |
| Sequence 579 | AA312025 |
| Sequence 580 | AA421510 |
| Sequence 581 | AA259166 |
| Sequence 582 | AA459831 |
| Sequence 583 | AA284410 |
| Sequence 584 | AA578295 |
| Sequence 585 | R20651 |
| Sequence 586 | AA364882 |
| Sequence 587 | AA135135 |
| Sequence 588 | AI332939 |
| Sequence 589 | W87865 |
| Sequence 590 | AA465598 |
| Sequence 591 | AA022478 |
| Sequence 592 | U72041 |
| Sequence 593 | AI239822 |
| Sequence 594 | AI253293 |
| Sequence 595 | AA206721 |
| Sequence 596 | AA447322 |
| Sequence 597 | AA044896 |
| Sequence 598 | AA149421 |
| Sequence 599 | AL047056 |
| Sequence 600 | AW057515 |
| Sequence 601 | AA774247 |
| Sequence 602 | AA412501 |
| Sequence 603 | AI458457 |
| Sequence 604 | AA307513 |
| Sequence 605 | AI761702 |
| Sequence 606 | AA400418 |
| Sequence 607 | AA524651 |
| Sequence 608 | AW102941 |
| Sequence 609 | AW188391 |
| Sequence 610 | T07196 |
| Sequence 611 | AI440194 |
| Sequence 612 | AI431350 |
| Sequence 613 | R60395 |
| Sequence 614 | AA263042 |
| Sequence 615 | AI267216 |
| Sequence 616 | AA027868 |
| Sequence 617 | AI357642 |
| Sequence 618 | AA430752 |
| Sequence 619 | AI827667 |
| Sequence 620 | AA135254 |
| Sequence 621 | AW067913 |
| Sequence 622 | AI914128 |
| Sequence 623 | AA430313 |
| Sequence 624 | AA047517 |
| Sequence 625 | AA075810 |
| Sequence 626 | AA001414 |
| Sequence 627 | AA375531 |
| Sequence 628 | R51416 |
| Sequence 629 | AA244066 |
| Sequence 630 | AA550927 |
| Sequence 631 | AA507791 |
| Sequence 632 | AI002745 |
| Sequence 633 | AA285290 |
| Sequence 634 | AI253379 |
| Sequence 635 | AA024480 |
| Sequence 636 | AA595047 |
| Sequence 637 | AA281307 |
| Sequence 638 | AA468760 |
| Sequence 639 | AI670913 |
| Sequence 640 | AA311639 |
| Sequence 641 | AI128986 |
| Sequence 642 | AA478891 |
| Sequence 643 | AA6I3935 |
| Sequence 644 | AA318009 |
| Sequence 645 | AA203537 |
| Sequence 646 | AA017262 |
| Sequence 647 | AA351443 |
| Sequence 648 | AA352938 |
| Sequence 649 | AA446322 |
| Sequence 650 | F12000 |
| Sequence 651 | AA001460 |
| Sequence 652 | AA346504 |
| Sequence 653 | AA779728 |
| Sequence 654 | AA349541 |
| Sequence 655 | AA115638 |
| Sequence 656 | AI050871 |
| Sequence 657 | AA827196 |
| Sequence 658 | AA393886 |
| Sequence 659 | AA083288 |
| Sequence 660 | AA302548 |
| Sequence 661 | AI085559 |
| Sequence 662 | AA812209 |
| Sequence 663 | AI253347 |
| Sequence 664 | AA082629 |
| Sequence 665 | AA121656 |
| Sequence 666 | R88370 |
| Sequence 667 | AA383100 |
| Sequence 668 | AI267353 |
| Sequence 669 | AA280873 |
| Sequence 670 | AI690986 |
| Sequence 671 | AA028008 |
| Sequence 672 | AA179664 |
| Sequence 673 | AI557338 |
| Sequence 674 | Z44402 |
| Sequence 675 | AI366374 |
| Sequence 676 | AI253335 |
| Sequence 677 | AL045332 |
| Sequence 678 | AA228953 |
| Sequence 679 | AA535837 |
| Sequence 680 | AW020820 |
| Sequence 681 | AI308072 |
| Sequence 682 | AA975275 |
| Sequence 683 | AI041116 |
| Sequence 684 | AI433757 |
| Sequence 685 | AI753951 |
| Sequence 686 | AA809784 |
| Sequence 687 | AA459476 |
| Sequence 688 | W37862 |
| Sequence 689 | AA206147 |
| Sequence 690 | AI697873 |
| Sequence 691 | AA311772 |
| Sequence 692 | AA447815 |
| Sequence 693 | AA312591 |
| Sequence 694 | AA216433 |
| Sequence 695 | AA622418 |
| Sequence 696 | AI922040 |
| Sequence 697 | AI589206 |
| Sequence 698 | AI928622 |
| Sequence 699 | AA485767 |
| Sequence 700 | AA070393 |
| Sequence 701 | AI216966 |
| Sequence 702 | AA639964 |
| Sequence 703 | AA204847 |
| Sequence 704 | AA491866 |
| Sequence 705 | AA227647 |
| Sequence 706 | AA448514 |
| Sequence 707 | AA171807 |
| Sequence 708 | AA765140 |
| Sequence 709 | AA172188 |
| Sequence 710 | AA029835 |
| Sequence 711 | AA907660 |
| Sequence 712 | AA770505 |
| Sequence 713 | AA070624 |
| Sequence 714 | AI139200 |
| Sequence 715 | AA025834 |
| Sequence 716 | AA504322 |
| Sequence 717 | AA307033 |
| Sequence 718 | AI269415 |
| Sequence 719 | AA345139 |
| Sequence 720 | AA361618 |
| Sequence 721 | AA627073 |
| Sequence 722 | R74152 |
| Sequence 723 | AA578773 |
| Sequence 724 | AA558636 |
| Sequence 725 | AA768951 |
| Sequence 726 | D79826 |
| Sequence 727 | H12519 |
| Sequence 728 | AA508889 |
| Sequence 729 | AL036423 |
| Sequence 730 | AA135065 |
| Sequence 731 | W93718 |
| Sequence 732 | AA022788 |
| Sequence 733 | AA358369 |
| Sequence 734 | F12883 |
| Sequence 735 | AA523902 |
| Sequence 736 | AA255526 |
| Sequence 737 | W94398 |
| Sequence 738 | AA435877 |
| Sequence 739 | T83709 |
| Sequence 740 | R14684 |
| Sequence 741 | AI671555 |
| Sequence 742 | AA037683 |
| Sequence 743 | AI188078 |
| Sequence 744 | AI817432 |
| Sequence 745 | AA364656 |
| Sequence 746 | AA489433 |
| Sequence 747 | AA037450 |
| Sequence 748 | AA460930 |
| Sequence 749 | AA166925 |
| Sequence 750 | N98749 |
| Sequence 751 | AA417324 |
| Sequence 752 | AA620750 |
| Sequence 753 | AA744849 |
| Sequence 754 | AA507041 |
| Sequence 755 | AI040234 |
| Sequence 756 | AA228940 |
| Sequence 757 | F05508 |
| Sequence 758 | C04925 |
| Sequence 759 | AA662702 |
| Sequence 760 | AI630144 |
| Sequence 761 | T72376 |
| Sequence 762 | AA225438 |
| Sequence 763 | AA190674 |
| Sequence 764 | AA070031 |
| Sequence 765 | AA526368 |
| Sequence 766 | AA357593 |
| Sequence 767 | AA527730 |
| Sequence 768 | W26780 |
| Sequence 769 | AA002131 |
| Sequence 770 | AI332588 |
| Sequence 771 | R92548 |
| Sequence 772 | AA229713 |
| Sequence 773 | AA018886 |
| Sequence 774 | AA531221 |
| Sequence 775 | AA188396 |
| Sequence 776 | AA452412 |
| Sequence 777 | AA112710 |
| Sequence 778 | AA385204 |
| Sequence 779 | AA295737 |
| Sequence 780 | AA224269 |
| Sequence 781 | AI708364 |
| Sequence 782 | AA442352 |
| Sequence 783 | AA725525 |
| Sequence 784 | AA418403 |
| Sequence 785 | AA228845 |
| Sequence 786 | AA453550 |
| Sequence 787 | AA522664 |
| Sequence 788 | AI133612 |
| Sequence 789 | AW138413 |
| Sequence 790 | AI216986 |
| Sequence 791 | AI638492 |
| Sequence 792 | AW103377 |
| Sequence 793 | AA486413 |
| Sequence 794 | AA019152 |
| Sequence 795 | H64556 |
| Sequence 796 | AA670280 |
| Sequence 797 | AA370976 |
| Sequence 798 | AA502798 |
| Sequence 799 | AA446366 |
| Sequence 800 | N67168 |
| Sequence 801 | W04240 |
| Sequence 802 | AA126750 |
| Sequence 803 | H67975 |
| Sequence 804 | AI005449 |
| Sequence 805 | AA947511 |
| Sequence 806 | AL120396 |
| Sequence 807 | AA229495 |
| Sequence 808 | AA324252 |
| Sequence 809 | AA354245 |
| Sequence 810 | AA314028 |
| Sequence 811 | AA574248 |
| Sequence 812 | AA569482 |
| Sequence 813 | AA226321 |
| Sequence 814 | AI025519 |
| Sequence 815 | AA724392 |
| Sequence 816 | AA125809 |
| Sequence 817 | AA843176 |
| Sequence 818 | AW023001 |
| Sequence 819 | AI338190 |
| Sequence 820 | AI817242 |
| Sequence 821 | AI521964 |
| Sequence 822 | AA325580 |
| Sequence 823 | AA347577 |
| Sequence 824 | D56420 |
| Sequence 825 | AA641520 |
| Sequence 826 | AI678281 |
| Sequence 827 | W27526 |
| Sequence 828 | AI380812 |
| Sequence 829 | M625145 |
| Sequence 830 | AA573974 |
| Sequence 831 | AI525651 |
| Sequence 832 | AI791719 |
| Sequence 833 | N40717 |
| Sequence 834 | AI694767 |
| Sequence 835 | AA417643 |
| Sequence 836 | AI989530 |
| Sequence 837 | AI755142 |
| Sequence 838 | AA633439 |
| Sequence 839 | AA847597 |
| Sequence 840 | AI312562 |
| Sequence 841 | AA931666 |
| Sequence 842 | AA742701 |
| Sequence 843 | R69818 |
| Sequence 844 | AI379679 |
| Sequence 845 | W19759 |
| Sequence 846 | AA683080 |
| Sequence 847 | AA972883 |
| Sequence 848 | AA722090 |
| Sequence 849 | AI267490 |
| Sequence 850 | AI110836 |
| Sequence 851 | AI064763 |
| Sequence 852 | AA627785 |
| Sequence 853 | AL036650 |
| Sequence 854 | AA278473 |
| Sequence 855 | AA515109 |
| Sequence 856 | AL119859 |
| Sequence 857 | AA280221 |
| Sequence 858 | AW088547 |
| Sequence 859 | AW178543 |
| Sequence 860 | AI826504 |
| Sequence 861 | AI754874 |
| Sequence 862 | AI948822 |
| Sequence 863 | AA188875 |
| Sequence 864 | AA988011 |
| Sequence 865 | AA489965 |
| Sequence 866 | AA946679 |
| Sequence 867 | AA522850 |
| Sequence 868 | AI813724 |
| Sequence 869 | AI740796 |
| Sequence 870 | AA317662 |
| Sequence 871 | AA099878 |
| Sequence 872 | AA372911 |
| Sequence 873 | T66793 |
| Sequence 874 | AA469129 |
| Sequence 875 | AI806044 |
| Sequence 876 | AI018728 |
| Sequence 877 | AA315780 |
| Sequence 878 | AA314804 |
| Sequence 879 | AI003782 |
| Sequence 880 | AI284549 |
| Sequence 881 | AA133452 |
| Sequence 882 | AA315621 |
| Sequence 883 | AA075012 |
| Sequence 884 | N41773 |
| Sequence 885 | R14689 |
| Sequence 886 | AA364190 |
| Sequence 887 | AA044750 |
| Sequence 888 | AA115588 |
| Sequence 889 | AA224994 |
| Sequence 890 | AA091445 |
| Sequence 891 | AA314920 |
| Sequence 892 | AA689334 |
| Sequence 893 | W03366 |
| Sequence 894 | AA405068 |
| Sequence 895 | AA417904 |
| Sequence 896 | AA347085 |
| Sequence 897 | AA702738 |
| Sequence 898 | AA088606 |
| Sequence 899 | AA528202 |
| Sequence 900 | C75042 |
| Sequence 901 | H92683 |
| Sequence 902 | AA602675 |
| Sequence 903 | AA146941 |
| Sequence 904 | AA923172 |
| Sequence 905 | AI470259 |
| Sequence 906 | AA722204 |
| Sequence 907 | F06621 |
| Sequence 908 | AA090790 |
| Sequence 909 | H05326 |
| Sequence 910 | AA328446 |
| Sequence 911 | AA298549 |
| Sequence 912 | AL036170 |
| Sequence 913 | T06574 |
| Sequence 914 | AA298285 |
| Sequence 915 | AA326242 |
| Sequence 916 | C20630 |
| Sequence 917 | AA226266 |
| Sequence 918 | AA486041 |
| Sequence 919 | AA225154 |
| Sequence 920 | F09972 |
| Sequence 921 | AI590351 |
| Sequence 922 | AA301408 |
| Sequence 923 | AA505423 |
| Sequence 924 | AI693302 |
| Sequence 925 | H09642 |
| Sequence 926 | AA146589 |
| Sequence 927 | AA600974 |
| Sequence 928 | AA044599 |
| Sequence 929 | AA243142 |
| Sequence 930 | AI625864 |
| Sequence 931 | AA057867 |
| Sequence 932 | Z44556 |
| Sequence 933 | AA112441 |
| Sequence 934 | AA040135 |
| Sequence 935 | AA480124 |
| Sequence 936 | AA233943 |
| Sequence 937 | AA306902 |
| Sequence 938 | AA319070 |
| Sequence 939 | AA083370 |
| Sequence 940 | AI458823 |
| Sequence 941 | AI168205 |
| Sequence 942 | AA935798 |
| Sequence 943 | AA630325 |
| Sequence 944 | AA069880 |
| Sequence 945 | AA707511 |
| Sequence 946 | AI263110 |
| Sequence 947 | AA713530 |
| Sequence 948 | AA284064 |
| Sequence 949 | AA576669 |
| Sequence 950 | AA664798 |
| Sequence 951 | AI696488 |
| Sequence 952 | AI758378 |
| Sequence 953 | AI741143 |
| Sequence 954 | AA430271 |
| Sequence 955 | AI754461 |
| Sequence 956 | AI280935 |
| Sequence 957 | AA541813 |
| Sequence 958 | AA377796 |
| Sequence 959 | C02739 |
| Sequence 960 | AA165562 |
| Sequence 961 | AL036801 |
| Sequence 962 | AA045870 |
| Sequence 963 | AA300869 |
| Sequence 964 | AA305030 |
| Sequence 965 | AA761069 |
| Sequence 966 | AA641623 |
| Sequence 967 | AI246615 |
| Sequence 968 | AA299140 |
| Sequence 969 | AA847452 |
| Sequence 970 | R50277 |
| Sequence 971 | AA252436 |
| Sequence 972 | AA016116 |
| Sequence 973 | AA909045 |
| Sequence 974 | AA713517 |
| Sequence 975 | AA743453 |
| Sequence 976 | AL048852 |
| Sequence 977 | AI298459 |
| Sequence 978 | N40598 |
| Sequence 979 | AW173161 |
| Sequence 980 | AA378508 |
| Sequence 981 | AA548465 |
| Sequence 982 | AA777529 |
| Sequence 983 | AAI76979 |
| Sequence 984 | AA506459 |
| Sequence 985 | AA306232 |
| Sequence 986 | AA280917 |
| Sequence 987 | AI690979 |
| Sequence 988 | AI880872 |
| Sequence 989 | T30767 |
| Sequence 990 | AI174822 |
| Sequence 991 | AI674871 |
| Sequence 992 | AA838697 |
| Sequence 993 | AA432264 |
| Sequence 994 | AI199681 |
| Sequence 995 | AA044586 |
| Sequence 996 | N67109 |
| Sequence 997 | AA169493 |
| Sequence 998 | AI554590 |
| Sequence 999 | R59789 |
| Sequence 1000 | AA305333 |
| Sequence 1001 | AW117546 |
| Sequence 1002 | AA029403 |
| Sequence 1003 | AI248187 |
| Sequence 1004 | AI370846 |
| Sequence 1005 | AA069801 |
| Sequence 1006 | AL120694 |
| Sequence 1007 | H83524 |
| Sequence 1008 | AI948503 |
| Sequence 1009 | AA631437 |
| Sequence 1010 | AI744509 |
| Sequence 1011 | AI050966 |
| Sequence 1012 | AA418177 |
| Sequence 1013 | AA644624 |
| Sequence 1014 | AA025551 |
| Sequence 1015 | R42583 |
| Sequence 1016 | AI357346 |
| Sequence 1017 | AL040518 |
| Sequence 1018 | AI627356 |
| Sequence 1019 | AA056058 |
| Sequence 1020 | AA993414 |
| Sequence 1021 | AW129657 |
| Sequence 1022 | AA399265 |
| Sequence 1023 | AA102274 |
| Sequence 1024 | N28426 |
| Sequence 1025 | AA069407 |
| Sequence 1026 | AA652478 |
| Sequence 1027 | AA126951 |
| Sequence 1028 | AA813266 |
| Sequence 1029 | AI309121 |
| Sequence 1030 | AA743710 |
| Sequence 1031 | AA229424 |
| Sequence 1032 | AA334754 |
| Sequence 1033 | AA401469 |
| Sequence 1034 | AL046756 |
| Sequence 1035 | AA680052 |
| Sequence 1036 | AA632291 |
| Sequence 1037 | AA349192 |
| Sequence 1038 | AI948513 |
| Sequence 1039 | AA772465 |
| Sequence 1040 | AA129772 |
| Sequence 1041 | AI270536 |
| Sequence 1042 | AI124856 |
| Sequence 1043 | AA244071 |
| Sequence 1044 | AI253319 |
| Sequence 1045 | AI188850 |
| Sequence 1046 | AA083300 |
| Sequence 1047 | AA977226 |
| Sequence 1048 | AA121615 |
| Sequence 1049 | AI955087 |
| Sequence 1050 | AI332459 |
| Sequence 1051 | AA506299 |
| Sequence 1052 | AI434180 |
| Sequence 1053 | AA102698 |
| Sequence 1054 | AA594131 |
| Sequence 1055 | AA806057 |
| Sequence 1056 | AI683431 |
| Sequence 1057 | AAI81017 |
| Sequence 1058 | W37322 |
| Sequence 1059 | AI949242 |
| Sequence 1060 | AA148087 |
| Sequence 1061 | AA053917 |
| Sequence 1062 | AA837048 |
| Sequence 1063 | AI743954 |
| Sequence 1064 | AI623891 |
| Sequence 1065 | AA292011 |
| Sequence 1066 | Z78396 |
| Sequence 1067 | Z30311 |
| Sequence 1068 | AA532663 |
| Sequence 1069 | AA298464 |
| Sequence 1070 | AI064691 |
| Sequence 1071 | AI500531 |
| Sequence 1072 | AA148268 |
| Sequence 1073 | AA826464 |
| Sequence 1074 | AI094803 |
| Sequence 1075 | AI971655 |
| Sequence 1076 | H03785 |
| Sequence 1077 | AA120953 |
| Sequence 1078 | AA503078 |
| Sequence 1079 | AI767430 |
| Sequence 1080 | AA236720 |
| Sequence 1081 | AA524672 |
| Sequence 1082 | AA349464 |
| Sequence 1083 | AI914133 |
| Sequence 1084 | AA884119 |
| Sequence 1085 | AA278475 |
| Sequence 1086 | AI865716 |
| Sequence 1087 | AA039812 |
| Sequence 1088 | AA376847 |
| Sequence 1089 | N63784 |
| Sequence 1090 | AA187426 |
| Sequence 1091 | AA228273 |
| Sequence 1092 | AI143623 |
| Sequence 1093 | AW169473 |
| Sequence 1094 | AA205647 |
| Sequence 1095 | AI755085 |
| Sequence 1096 | AI627945 |
| Sequence 1097 | AA599864 |
| Sequence 1098 | AA403109 |
| Sequence 1099 | AA476756 |
| Sequence 1100 | AA532883 |
| Sequence 1101 | AA305697 |
| Sequence 1102 | AI355050 |
| Sequence 1103 | C06419 |
| Sequence 1104 | AA247232 |
| Sequence 1105 | AI092766 |
| Sequence 1106 | AA705269 |
| Sequence 1107 | AA345925 |
| Sequence 1108 | AA010828 |
| Sequence 1109 | AI302083 |
| Sequence 1110 | AA011000 |
| Sequence 1111 | AI080480 |
| Sequence 1112 | AA984407 |
| Sequence 1113 | AA405758 |
| Sequence 1114 | AI373634 |
| Sequence 1115 | AI459234 |
| Sequence 1116 | AA481476 |
| Sequence 1117 | AI278611 |
| Sequence 1118 | M78906 |
| Sequence 1119 | AI031653 |
| Sequence 1120 | AF150208 |
| Sequence 1121 | AA525487 |
| Sequence 1122 | AA351741 |
| Sequence 1123 | AW073731 |
| Sequence 1124 | AA430160 |
| Sequence 1125 | AI084005 |
| Sequence 1126 | AA157733 |
| Sequence 1127 | AA334735 |
| Sequence 1128 | AA148206 |
| Sequence 1129 | AA461260 |
| Sequence 1130 | AI686139 |
| Sequence 1131 | AA314161 |
| Sequence 1132 | AA307716 |
| Sequence 1133 | AA592908 |
| Sequence 1134 | AI693674 |
| Sequence 1135 | AI022340 |
| Sequence 1136 | AI802946 |
| Sequence 1137 | AA479284 |
| Sequence 1138 | AI689722 |
| Sequence 1139 | AA085283 |
| Sequence 1140 | AA215800 |
| Sequence 1141 | AL043232 |
| Sequence 1142 | AI869322 |
| Sequence 1143 | AA773478 |
| Sequence 1144 | AA968556 |
| Sequence 1145 | AA481979 |
| Sequence 1146 | AA608749 |
| Sequence 1147 | AA521213 |
| Sequence 1148 | AA430754 |
| Sequence 1149 | AA917666 |
| Sequence 1150 | AI743852 |
| Sequence 1151 | AA004042 |
| Sequence 1152 | AA531563 |
| Sequence 1153 | AA280753 |
| Sequence 1154 | AA906868 |
| Sequence 1155 | AA479781 |
| Sequence 1156 | AA446116 |
| Sequence 1157 | AA348116 |
| Sequence 1158 | AI080439 |
| Sequence 1159 | AI825988 |
| Sequence 1160 | AI267321 |
| Sequence 1161 | AA679068 |
| Sequence 1162 | AI557336 |
| Sequence 1163 | AA460440 |
| Sequence 1164 | AA308091 |
| Sequence 1165 | AA513597 |
| Sequence 1166 | AA442287 |
| Sequence 1167 | AW084125 |
| Sequence 1168 | AI274700 |
| Sequence 1169 | AW138181 |
| Sequence 1170 | AA576425 |
| Sequence 1171 | AA304651 |
| Sequence 1172 | AI285435 |
| Sequence 1173 | AI279882 |
| Sequence 1174 | AA313778 |
| Sequence 1175 | AI366381 |
| Sequence 1176 | AI340044 |
| Sequence 1177 | AL039293 |
| Sequence 1178 | C18181 |
| Sequence 1179 | AA449649 |
| Sequence 1180 | AA312605 |
| Sequence 1181 | AA046577 |
| Sequence 1182 | AI075037 |
| Sequence 1183 | AI421027 |
| Sequence 1184 | AA781086 |
| Sequence 1185 | AA825512 |
| Sequence 1186 | AA205546 |
| Sequence 1187 | AA027882 |
| Sequence 1188 | AA810418 |
| Sequence 1189 | AA348878 |
| Sequence 1190 | AA281653 |
| Sequence 1191 | H17170 |
| Sequence 1192 | AA195751 |
| Sequence 1193 | AA364261 |
| Sequence 1194 | AA235838 |
| Sequence 1195 | AA503943 |
| Sequence 1196 | Z25169 |
| Sequence 1197 | AA971698 |
| Sequence 1198 | AA022937 |
| Sequence 1199 | AA255974 |
| Sequence 1200 | AA625467 |
| Sequence 1201 | AA602662 |
| Sequence 1202 | AI420026 |
| Sequence 1203 | AA215538 |
| Sequence 1204 | AI133208 |
| Sequence 1205 | R11114 |
| Sequence 1206 | AA479689 |
| Sequence 1207 | AA528104 |
| Sequence 1208 | AA034912 |
| Sequence 1209 | AA243835 |
| Sequence 1210 | AW003247 |
| Sequence 1211 | AA479276 |
| Sequence 1212 | AA307209 |
| Sequence 1213 | AI332712 |
| Sequence 1214 | AA702942 |
| Sequence 1215 | AW163314 |
| Sequence 1216 | AA336412 |
| Sequence 1217 | H58571 |
| Sequence 1218 | AA186364 |
| Sequence 1219 | AA398463 |
| Sequence 1220 | AA017500 |
| Sequence 1221 | AW088608 |
| Sequence 1222 | AA493183 |
| Sequence 1223 | AI240899 |
| Sequence 1224 | R55600 |
| Sequence 1225 | AA464568 |
| Sequence 1226 | AF001542 |
| Sequence 1227 | AA384987 |
| Sequence 1228 | AA188771 |
| Sequence 1229 | AA234050 |
| Sequence 1230 | AI557495 |
| Sequence 1231 | AW080196 |
| Sequence 1232 | AA418719 |
| Sequence 1233 | AA225294 |
| Sequence 1234 | AA812993 |
| Sequence 1235 | AA312689 |
| Sequence 1236 | H13891 |
| Sequence 1237 | T77631 |
| Sequence 1238 | AA311905 |
| Sequence 1239 | AI203546 |
| Sequence 1240 | AI435606 |
| Sequence 1241 | AA143170 |
| Sequence 1242 | AL037316 |
| Sequence 1243 | AI205868 |
| Sequence 1244 | AW103458 |
| Sequence 1245 | W90519 |
| Sequence 1246 | AI367641 |
| Sequence 1247 | AA394298 |
| Sequence 1248 | AA167826 |
| Sequence 1249 | AA322540 |
| Sequence 1250 | AI253192 |
| Sequence 1251 | AA843531 |
| Sequence 1252 | AA203110 |
| Sequence 1253 | AI690278 |
| Sequence 1254 | AA506323 |
| Sequence 1255 | AA406478 |
| Sequence 1256 | AI816058 |
| Sequence 1257 | AA149219 |
| Sequence 1258 | AW176020 |
| Sequence 1259 | AA507792 |
| Sequence 1260 | AA468839 |
| Sequence 1261 | M195151 |
| Sequence 1262 | AA070277 |
| Sequence 1263 | T08287 |
| Sequence 1264 | AA130555 |
| Sequence 1265 | AL122121 |
| Sequence 1266 | AF112481 |
| Sequence 1267 | AF05I334 |
| Sequence 1268 | U61397 |
| Sequence 1269 | AF116827 |
| Sequence 1270 | AB029020 |
| Sequence 1271 | AL110183 |
| Sequence 1272 | AB020636 |
| Sequence 1273 | AF000986 |
| Sequence 1274 | D86326 |
| Sequence 1275 | AJ005016 |
| Sequence 1276 | AB020637 |
| Sequence 1277 | AL110159 |
| Sequence 1278 | D21209 |
| Sequence 1279 | L07077 |
| Sequence 1280 | X74801 |
| Sequence 1281 | AB015333 |
| Sequence 1282 | AB011119 |
| Sequence 1283 | Y11395 |
| Sequence 1284 | AB029028 |
| Sequence 1285 | AF054179 |
| Sequence 1286 | M21895 |
| Sequence 1287 | AL080206 |
| Sequence 1288 | AL050355 |
| Sequence 1289 | AB006965 |
| Sequence 1290 | AL117563 |
| Sequence 1291 | X75861 |
| Sequence 1292 | D13315 |
| Sequence 1293 | D50420 |
| Sequence 1294 | S39329 |
| Sequence 1295 | D87444 |
| Sequence 1296 | AL117412 |
| Sequence 1297 | AF083217 |
| Sequence 1298 | U51903 |
| Sequence 1299 | AB014547 |
| Sequence 1300 | U14528 |
| Sequence 1301 | AF153608 |
| Sequence 1302 | U57847 |
| Sequence 1303 | AL049276 |
| Sequence 1304 | AF124438 |
| Sequence 1305 | AF007144 |
| Sequence 1306 | L01100 |
| Sequence 1307 | AF056617 |
| Sequence 1308 | U57877 |
| Sequence 1309 | X74331 |
| Sequence 1310 | S82081 |
| Sequence 1311 | AF115402 |
| Sequence 1312 | M29366 |
| Sequence 1313 | X04106 |
| Sequence 1314 | AF151103 |
| Sequence 1315 | S75755 |
| Sequence 1316 | X76057 |
| Sequence 1317 | AF070641 |
| Sequence 1318 | X78933 |
| Sequence 1319 | AF006083 |
| Sequence 1320 | S73498 |
| Sequence 1321 | AB022663 |
| Sequence 1322 | X94910 |
| Sequence 1323 | X01742 |
| Sequence 1324 | AB020633 |
| Sequence 1325 | D14043 |
| Sequence 1326 | L10284 |
| Sequence 1327 | X15729 |
| Sequence 1328 | AB020680 |
| Sequence 1329 | X59892 |
| Sequence 1330 | U77396 |
| Sequence 1331 | U84138 |
| Sequence 1332 | AB007873 |
| Sequence 1333 | D87442 |
| Sequence 1334 | D42045 |
| Sequence 1335 | AF008442 |
| Sequence 1336 | AJ002955 |
| Sequence 1337 | M94856 |
| Sequence 1338 | X16940 |
| Sequence 1339 | M36634 |
| Sequence 1340 | AB018266 |
| Sequence 1341 | M17323 |
| Sequence 1342 | X75593 |
| Sequence 1343 | J04137 |
| Sequence 1344 | AL049969 |
| Sequence 1345 | D13641 |
| Sequence 1346 | D31886 |
| Sequence 1347 | X63563 |
| Sequence 1348 | D63480 |
| Sequence 1349 | AF039918 |
| Sequence 1350 | X80910 |
| Sequence 1351 | D87969 |
| Sequence 1352 | Z24724 |
| Sequence 1353 | U47674 |
| Sequence 1354 | AB011158 |
| Sequence 1355 | AL050071 |
| Sequence 1356 | AB014610 |
| Sequence 1357 | AL050290 |
| Sequence 1358 | AD001528 |
| Sequence 1359 | U67784 |
| Sequence 1360 | L09749 |
| Sequence 1361 | X85129 |
| Sequence 1362 | AF022913 |
| Sequence 1363 | AF131738 |
| Sequence 1364 | AJ004913 |
| Sequence 1365 | AB002383 |
| Sequence 1366 | J03015 |
| Sequence 1367 | D29954 |
| Sequence 1368 | AF069469 |
| Sequence 1369 | AL049996 |
| Sequence 1370 | D42044 |
| Sequence 1371 | AL080181 |
| Sequence 1372 | AF061258 |
| Sequence 1373 | AL110153 |
| Sequence 1374 | AL080202 |
| Sequence 1375 | U19769 |
| Sequence 1376 | AB003103 |
| Sequence 1377 | L13848 |
| Sequence 1378 | M24194 |
| Sequence 1379 | J05480 |
| Sequence 1380 | M16342 |
| Sequence 1381 | AB002365 |
| Sequence 1382 | AF144566 |
| Sequence 1383 | AJ001443 |
| Sequence 1384 | U54559 |
| Sequence 1385 | AF075061 |
| Sequence 1386 | U37143 |
| Sequence 1387 | D79986 |
| Sequence 1388 | AF013758 |
| Sequence 1389 | AB020694 |
| Sequence 1390 | AF103804 |
| Sequence 1391 | AF052578 |
| Sequence 1392 | AL117599 |
| Sequence 1393 | AL110211 |
| Sequence 1394 | AB018342 |
| Sequence 1395 | AF151844 |
| Sequence 1396 | AL050192 |
| Sequence 1397 | X97065 |
| Sequence 1398 | L12168 |
| Sequence 1399 | J03143 |
| Sequence 1400 | X59405 |
| Sequence 1401 | X76732 |
| Sequence 1402 | U36336 |
| Sequence 1403 | AF131802 |
| Sequence 1404 | AB019435 |
| Sequence 1405 | L41143 |
| Sequence 1406 | AF141327 |
| Sequence 1407 | U80034 |
| Sequence 1408 | AF062318 |
| Sequence 1409 | Y13834 |
| Sequence 1410 | AB024518 |
| Sequence 1411 | AB007898 |
| Sequence 1412 | U58855 |
| Sequence 1413 | K03515 |
| Sequence 1414 | M81757 |
| Sequence 1415 | M60527 |
| Sequence 1416 | AL110181 |
| Sequence 1417 | M25756 |
| Sequence 1418 | AB033061 |
| Sequence 1419 | D31770 |
| Sequence 1420 | AB004066 |
| Sequence 1421 | E01932 |
| Sequence 1422 | U90909 |
| Sequence 1423 | AF078864 |
| Sequence 1424 | AF047020 |
| Sequence 1425 | J04183 |
| Sequence 1426 | AL050089 |
| Sequence 1427 | X57025 |
| Sequence 1428 | D14710 |
| Sequence 1429 | AI8585 |
| Sequence 1430 | AL049932 |
| Sequence 1431 | Z50749 |
| Sequence 1432 | AB014579 |
| Sequence 1433 | AF000652 |
| Sequence 1434 | X03559 |
| Sequence 1435 | L37080 |
| Sequence 1436 | M29548 |
| Sequence 1437 | AB000468 |
| Sequence 1438 | AF061261 |
| Sequence 1439 | AF126780 |
| Sequence 1440 | X77196 |
| Sequence 1441 | AB007949 |
| Sequence 1442 | D49490 |
| Sequence 1443 | AF006086 |
| Sequence 1444 | U39657 |
| Sequence 1445 | AF005043 |
| Sequence 1446 | AF053535 |
| Sequence 1447 | AL110141 |
| Sequence 1448 | AF151805 |
| Sequence 1449 | AB033049 |
| Sequence 1450 | L08437 |
| Sequence 1451 | AF020038 |
| Sequence 1452 | M74777 |
| Sequence 1453 | L31801 |
| Sequence 1454 | X60708 |
| Sequence 1455 | AF052124 |
| Sequence 1456 | AF077202 |
| Sequence 1457 | E01915 |
| Sequence 1458 | M22865 |
| Sequence 1459 | M73837 |
| Sequence 1460 | U72937 |
| Sequence 1461 | S72481 |
| Sequence 1462 | AB020724 |
| Sequence 1463 | AL117621 |
| Sequence 1464 | AB019568 |
| Sequence 1465 | L05425 |
| Sequence 1466 | AL122097 |
| Sequence 1467 | AF1511868 |
| Sequence 1468 | AF085355 |
| Sequence 1469 | AL049471 |
| Sequence 1470 | AL117623 |
| Sequence 1471 | AB007956 |
| Sequence 1472 | U88666 |
| Sequence 1473 | AF020797 |
| Sequence 1474 | U30313 |
| Sequence 1475 | U16850 |
| Sequence 1476 | AF032456 |
| Sequence 1477 | AB026723 |
| Sequence 1478 | AF027515 |
| Sequence 1479 | U58913 |
| Sequence 1480 | AL122084 |
| Sequence 1481 | Y09305 |
| Sequence 1482 | M14648 |
| Sequence 1483 | AF126181 |
| Sequence 1484 | L14778 |
| Sequence 1485 | D87845 |
| Sequence 1486 | AF047489 |
| Sequence 1487 | D26485 |
| Sequence 1488 | X73608 |
| Sequence 1489 | U64820 |
| Sequence 1490 | D89053 |
| Sequence 1491 | AF012073 |
| Sequence 1492 | AJ002744 |
| Sequence 1493 | D31883 |
| Sequence 1494 | U01923 |
| Sequence 1495 | AB018331 |
| Sequence 1496 | AB019563 |
| Sequence 1497 | U41813 |
| Sequence 1498 | M61199 |
| Sequence 1499 | M22538 |
| Sequence 1500 | AF025998 |
| Sequence 1501 | AL049944 |
| Sequence 1502 | AB007963 |
| Sequence 1503 | AB006968 |
| Sequence 1504 | AF077037 |
| Sequence 1505 | U43701 |
| Sequence 1506 | E05957 |
| Sequence 1507 | AF103907 |
| Sequence 1508 | M74002 |
| Sequence 1509 | D87735 |
| Sequence 1510 | L17128 |
| Sequence 1511 | D21163 |
| Sequence 1512 | AF070523 |
| Sequence 1513 | AB011148 |
| Sequence 1514 | L13385 |
| Sequence 1515 | D13988 |
| Sequence 1516 | AL110238 |
| Sequence 1517 | D21210 |
| Sequence 1518 | AB011087 |
| Sequence 1519 | AF054284 |
| Sequence 1520 | AF176702 |
| Sequence 1521 | AB004857 |
| Sequence 1522 | D28589 |
| Sequence 1523 | AF035293 |
| Sequence 1524 | U53328 |
| Sequence 1525 | X97324 |
| Sequence 1526 | M24902 |
| Sequence 1527 | AF001433 |
| Sequence 1528 | AF092138 |
| Sequence 1529 | AF131775 |
| Sequence 1530 | AB023224 |
| Sequence 1531 | AF084555 |
| Sequence 1532 | D83032 |
| Sequence 1533 | AF038451 |
| Sequence 1534 | D00422 |
| Sequence 1535 | D13388 |
| Sequence 1536 | U51134 |
| Sequence 1537 | L16510 |
| Sequence 1538 | E01650 |
| Sequence 1539 | E02628 |
| Sequence 1540 | U62434 |
| Sequence 1541 | J02923 |
| Sequence 1542 | U60975 |
| Sequence 1543 | U79278 |
| Sequence 1544 | AF167572 |
| Sequence 1545 | AF169797 |
| Sequence 1546 | M23161 |
| Sequence 1547 | M33197 |
| Sequence 1548 | D87682 |
| Sequence 1549 | AF125392 |
| Sequence 1550 | AF064257 |
| Sequence 1551 | U00946 |
| Sequence 1552 | S70290 |
| Sequence 1553 | AB019691 |
| Sequence 1554 | J03537 |
| Sequence 1555 | D38305 |
| Sequence 1556 | D14697 |
| Sequence 1557 | AF147354 |
| Sequence 1558 | AB018330 |
| Sequence 1559 | D50372 |
| Sequence 1560 | D12676 |
| Sequence 1561 | AF098297 |
| Sequence 1562 | X81900 |
| Sequence 1563 | X56932 |
| Sequence 1564 | U53468 |
| Sequence 1565 | AF165281 |
| Sequence 1566 | U12979 |
| Sequence 1567 | AF188746 |
| Sequence 1568 | D49489 |
| Sequence 1569 | M24630 |
| Sequence 1570 | D31885 |
| Sequence 1571 | AL117441 |
| Sequence 1572 | M21896 |
| Sequence 1573 | S78203 |
| Sequence 1574 | AF004561 |
| Sequence 1575 | U21936 |
| Sequence 1576 | D86985 |
| Sequence 1577 | AL050064 |
| Sequence 1578 | AB028956 |
| Sequence 1579 | E07798 |
| Sequence 1580 | AB023230 |
| Sequence 1581 | AB023182 |
| Sequence 1582 | AB028948 |
| Sequence 1583 | AF054182 |
| Sequence 1584 | AF091263 |
| Sequence 1585 | U10439 |
| Sequence 1586 | L49399 |
| Sequence 1587 | D50645 |
| Sequence 1588 | AF131749 |
| Sequence 1589 | AF013759 |
| Sequence 1590 | AF097535 |
| Sequence 1591 | Y12860 |
| Sequence 1592 | X60489 |
| Sequence 1593 | Z48042 |
| Sequence 1594 | U79751 |
| Sequence 1595 | U81602 |
| Sequence 1596 | L19161 |
| Sequence 1597 | D13866 |
| Sequence 1598 | U02493 |
| Sequence 1599 | U03274 |
| Sequence 1600 | Z36785 |
| Sequence 1601 | AF070655 |
| Sequence 1602 | J04794 |
| Sequence 1603 | AF039029 |
| Sequence 1604 | Z22534 |
| Sequence 1605 | M10036 |
| Sequence 1606 | AF131831 |
| Sequence 1607 | AF068754 |
| Sequence 1608 | AL049367 |
| Sequence 1609 | X53793 |
| Sequence 1610 | AF070553 |
| Sequence 1611 | AB018281 |
| Sequence 1612 | U39064 |
| Sequence 1613 | U17714 |
| Sequence 1614 | U42412 |
| Sequence 1615 | D43947 |
| Sequence 1616 | AF100759 |
| Sequence 1617 | AB019564 |
| Sequence 1618 | D63998 |
| Sequence 1619 | AL050265 |
| Sequence 1620 | AB033078 |
| Sequence 1621 | X77548 |
| Sequence 1622 | U00947 |
| Sequence 1623 | X94323 |
| Sequence 1624 | E03569 |
| Sequence 1625 | L25899 |
| Sequence 1626 | Z47553 |
| Sequence 1627 | AF155110 |
| Sequence 1628 | AF075587 |
| Sequence 1629 | AB021288 |
| Sequence 1630 | D14665 |
| Sequence 1631 | AB014598 |
| Sequence 1632 | X57398 |
| Sequence 1633 | D87450 |
| Sequence 1634 | AF132941 |
| Sequence 1635 | M21154 |
| Sequence 1636 | AI7362 |
| Sequence 1637 | AF070664 |
| Sequence 1638 | AF151873 |
| Sequence 1639 | D63486 |
| Sequence 1640 | AJ000644 |
| Sequence 1641 | L38961 |
| Sequence 1642 | AF042166 |
| Sequence 1643 | AF078847 |
| Sequence 1644 | AJ130733 |
| Sequence 1645 | X54326 |
| Sequence 1646 | J04208 |
| Sequence 1647 | AF188745 |
| Sequence 1648 | U18422 |
| Sequence 1649 | J02888 |
| Sequence 1650 | D63477 |
| Sequence 1651 | E03414 |
| Sequence 1652 | AB015639 |
| Sequence 1653 | AF040707 |
| Sequence 1654 | M61831 |
| Sequence 1655 | U39360 |
| Sequence 1656 | AL050255 |
| Sequence 1657 | U73824 |
| Sequence 1658 | Y00281 |
| Sequence 1659 | U41515 |
| Sequence 1660 | AF100761 |
| Sequence 1662 | AB007618 |
| Sequence 1663 | AF055994 |
| Sequence 1664 | AF047472 |
| Sequence 1665 | D78152 |
| Sequence 1666 | AJ237946 |
| Sequence 1667 | E08293 |
| Sequence 1668 | D28473 |
| Sequence 1669 | AB018358 |
| Sequence 1670 | AF048731 |
| Sequence 1671 | U90551 |
| Sequence 1672 | AF070561 |
| Sequence 1673 | M17885 |
| Sequence 1674 | M74524 |
| Sequence 1675 | AB000220 |
| Sequence 1676 | AB020639 |
| Sequence 1677 | AF053977 |
| Sequence 1678 | D88532 |
| Sequence 1679 | AF007216 |
| Sequence 1680 | X05332 |
| Sequence 1681 | U24105 |
| Sequence 1682 | AB002387 |
| Sequence 1683 | AB007957 |
| Sequence 1684 | AL117666 |
| Sequence 1685 | AL049951 |
| Sequence 1686 | L07540 |
| Sequence 1687 | AL110242 |
| Sequence 1688 | AF078861 |
| Sequence 1689 | AF153612 |
| Sequence 1690 | M17254 |
| Sequence 1691 | D86967 |
| Sequence 1692 | AB014548 |
| Sequence 1693 | U20157 |
| Sequence 1694 | AF022229 |
| Sequence 1695 | AL050051 |
| Sequence 1696 | AB033071 |
| Sequence 1697 | AF052093 |
| Sequence 1698 | Z13009 |
| Sequence 1699 | AF156965 |
| Sequence 1700 | AB018334 |
| Sequence 1701 | M14328 |
| Sequence 1702 | U17104 |
| Sequence 1703 | U10117 |
| Sequence 1704 | AL117567 |
| Sequence 1705 | AL080097 |
| Sequence 1706 | AF151840 |
| Sequence 1707 | U09848 |
| Sequence 1708 | AB001872 |
| Sequence 1709 | AL050373 |
| Sequence 1710 | AB018270 |
| Sequence 1711 | AF167160 |
| Sequence 1712 | AB033091 |
| Sequence 1713 | AB018296 |
| Sequence 1714 | M22918 |
| Sequence 1715 | L05186 |
| Sequence 1716 | Y12065 |
| Sequence 1717 | M85168 |
| Sequence 1718 | L11066 |
| Sequence 1719 | AF016270 |
| Sequence 1720 | D21262 |
| Sequence 1721 | M16660 |
| Sequence 1722 | AL117443 |
| Sequence 1723 | D50916 |
| Sequence 1724 | U68536 |
| Sequence 1725 | J05192 |
| Sequence 1726 | D87667 |
| Sequence 1727 | AB028973 |
| Sequence 1728 | AF106943 |
| Sequence 1729 | U18197 |
| Sequence 1730 | Y14738 |
| Sequence 1731 | AF159056 |
| Sequence 1732 | U48857 |
| Sequence 1733 | X04297 |
| Sequence 1734 | AB011165 |
| Sequence 1735 | AF097159 |
| Sequence 1736 | X15949 |
| Sequence 1737 | D84105 |
| Sequence 1738 | AF125096 |
| Sequence 1739 | AF053551 |
| Sequence 1740 | AF007149 |
| Sequence 1741 | AJ224326 |
| Sequence 1742 | M55543 |
| Sequence 1743 | AB032948 |
| Sequence 1744 | AF091076 |
| Sequence 1745 | AF065391 |
| Sequence 1746 | L13977 |
| Sequence 1747 | M87284 |
| Sequence 1748 | AB002382 |
| Sequence 1749 | Z82022 |
| Sequence 1750 | AF151908 |
| Sequence 1751 | AL050107 |
| Sequence 1752 | AF027824 |
| Sequence 1753 | AF144755 |
| Sequence 1754 | AL080201 |
| Sequence 1755 | AF089747 |
| Sequence 1756 | D13119 |
| Sequence 1757 | AF146277 |
| Sequence 1758 | AF170583 |
| Sequence 1759 | AL050298 |
| Sequence 1760 | M18366 |
| Sequence 1761 | Y00282 |
| Sequence 1762 | J04823 |
| Sequence 1763 | AL110214 |
| Sequence 1764 | AL080088 |
| Sequence 1765 | AF100755 |
| Sequence 1766 | AF078860 |
| Sequence 1767 | U03886 |
| Sequence 1768 | L07033 |
| Sequence 1769 | U07919 |
| Sequence 1770 | D13629 |
| Sequence 1771 | AF191018 |
| Sequence 1772 | X04697 |
| Sequence 1773 | X81109 |
| Sequence 1774 | AL079298 |
| Sequence 1775 | X56958 |
| Sequence 1776 | AL110126 |
| Sequence 1777 | X63679 |
| Sequence 1778 | X17620 |
| Sequence 1779 | A21185 |
| Sequence 1780 | AF087942 |
| Sequence 1781 | E01954 |
| Sequence 1782 | AF077200 |
| Sequence 1783 | D87078 |
| Sequence 1784 | AL050272 |
| Sequence 1785 | M21897 |
| Sequence 1786 | U90144 |
| Sequence 1787 | D14696 |
| Sequence 1788 | M20372 |
| Sequence 1789 | AF085845 |
| Sequence 1790 | AB033114 |
| Sequence 1791 | E01497 |
| Sequence 1792 | AB007510 |
| Sequence 1793 | D87666 |
| Sequence 1794 | D38524 |
| Sequence 1795 | E00882 |
| Sequence 1796 | X02875 |
| Sequence 1797 | AF085224 |
| Sequence 1798 | AF071076 |
| Sequence 1799 | AF102850 |
| Sequence 1800 | AB002369 |
| Sequence 1801 | AB011004 |
| Sequence 1802 | AL050289 |
| Sequence 1803 | S41458 |
| Sequence 1804 | AF088004 |
| Sequence 1805 | AF086431 |
| Sequence 1806 | AF010233 |
| Sequence 1807 | AF086336 |
| Sequence 1808 | AF057705 |
| Sequence 1809 | AB018280 |
| Sequence 1810 | AF016496 |
| Sequence 1811 | M26663 |
| Sequence 1812 | M17886 |
| Sequence 1813 | X07466 |
| Sequence 1814 | D31767 |
| Sequence 1815 | L05779 |
| Sequence 1816 | U07809 |
| Sequence 1817 | AF137334 |
| Sequence 1818 | AF056322 |
| Sequence 1819 | AB001636 |
| Sequence 1820 | D38555 |
| Sequence 1821 | D28540 |
| Sequence 1822 | AF191298 |
| Sequence 1823 | AF042346 |
| Sequence 1824 | E01956 |
| Sequence 1825 | X70394 |
| Sequence 1826 | U41060 |
| Sequence 1827 | AB000887 |
| Sequence 1828 | U25182 |
| Sequence 1829 | X87241 |
| Sequence 1830 | AB007972 |
| Sequence 1831 | M63573 |
| Sequence 1832 | X07270 |
| Sequence 1833 | AF124439 |
| Sequence 1834 | D50406 |
| Sequence 1835 | J04443 |
| Sequence 1836 | D21260 |
| Sequence 1837 | X15187 |
| Sequence 1838 | D89092 |
| Sequence 1839 | K03002 |
| Sequence 1840 | AF035286 |
| Sequence 1841 | M83822 |
| Sequence 1842 | M55409 |
| Sequence 1843 | AB007885 |
| Sequence 1844 | AL050198 |
| Sequence 1845 | M26481 |
| Sequence 1846 | M27544 |
| Sequence 1847 | X83544 |
| Sequence 1848 | D14812 |
| Sequence 1849 | AF056433 |
| Sequence 1850 | Z23064 |
| Sequence 1851 | AB032983 |
| Sequence 1852 | AL117514 |
| Sequence 1853 | X59131 |
| Sequence 1854 | AF073475 |
| Sequence 1855 | AF044954 |
| Sequence 1856 | X60221 |
| Sequence 1857 | AB007882 |
| Sequence 1858 | AF082513 |
| Sequence 1859 | AF071202 |
| Sequence 1860 | M26383 |
| Sequence 1861 | X57351 |
| Sequence 1862 | AF007142 |
| Sequence 1863 | AF020202 |
| Sequence 1864 | AB020686 |
| Sequence 1865 | X91257 |
| Sequence 1866 | D32051 |
| Sequence 1867 | AF147339 |
| Sequence 1868 | AB002306 |
| Sequence 1869 | AF100756 |
| Sequence 1870 | U01925 |
| Sequence 1871 | D14878 |
| Sequence 1872 | M22590 |
| Sequence 1873 | M93651 |
| Sequence 1874 | AF054838 |
| Sequence 1875 | M69066 |
| Sequence 1876 | AF017269 |
| Sequence 1877 | J03007 |
| Sequence 1878 | AF019214 |
| Sequence 1879 | AF039023 |
| Sequence 1880 | X66276 |
| Sequence 1881 | AL110179 |
| Sequence 1882 | D86984 |
| Sequence 1883 | D90226 |
| Sequence 1884 | AL035304 |
| Sequence 1885 | D13643 |
| Sequence 1886 | K01911 |
| Sequence 1887 | L28997 |
| Sequence 1888 | AB023173 |
| Sequence 1889 | Y00971 |
| Sequence 1890 | AB033899 |
| Sequence 1891 | U90550 |
| Sequence 1892 | M10119 |
| Sequence 1893 | AL133078 |
| Sequence 1894 | J04607 |
| Sequence 1895 | J02642 |
| Sequence 1896 | A06919 |
| Sequence 1897 | M34181 |
| Sequence 1898 | Z46973 |
| Sequence 1899 | AF038404 |
| Sequence 1900 | AF083441 |
| Sequence 1901 | AF107405 |
| Sequence 1902 | AF201077 |
| Sequence 1903 | U02680 |
| Sequence 1904 | L08599 |
| Sequence 1905 | AF019226 |
| Sequence 1906 | AF176574 |
| Sequence 1907 | AB028977 |
| Sequence 1908 | AJ000881 |
| Sequence 1909 | D89678 |
| Sequence 1910 | D50405 |
| Sequence 1911 | D29641 |
| Sequence 1912 | M34840 |
| Sequence 1913 | V62427 |
| Sequence 1914 | V62430 |
| Sequence 1915 | X20432 |
| Sequence 1916 | V87930 |
| Sequence 1917 | V58694 |
| Sequence 1918 | X98608 |
| Sequence 1919 | Z15132 |
| Sequence 1920 | V58628 |
| Sequence 1921 | D97526 |
| Sequence 1922 | X25445 |
| Sequence 1923 | X04318 |
| Sequence 1924 | X27262 |
| Sequence 1925 | T59274 |
| Sequence 1926 | V69358 |
| Sequence 1927 | X98998 |
| Sequence 1928 | Z10658 |
| Sequence 1929 | X76578 |
| Sequence 1930 | Z06652 |
| Sequence 1931 | T37414 |
| Sequence 1932 | V56670 |
| Sequence 1933 | V58682 |
| Sequence 1934 | Z15583 |
| Sequence 1935 | T18813 |
| Sequence 1936 | X22116 |
| Sequence 1937 | T61701 |
| Sequence 1938 | Z00842 |
| Sequence 1939 | V58756 |
| Sequence 1940 | V57903 |
| Sequence 1941 | X03841 |
| Sequence 1942 | Z17335 |
| Sequence 1943 | X26850 |
| Sequence 1944 | Z33658 |
| Sequence 1945 | Z14392 |
| Sequence 1946 | V63172 |
| Sequence 1947 | Z15729 |
| Sequence 1948 | V34236 |
| Sequence 1949 | AC31081 |
| Sequence 1950 | AC35659 |
| Sequence 1951 | AC36156 |
| Sequence 1952 | AC33865 |
| Sequence 1953 | AC36153 |
| Sequence 1954 | AC29851 |
| Sequence 1955 | AC36206 |
| Sequence 1956 | AC36220 |
| Sequence 1957 | AC36078 |
| Sequence 1958 | AC36205 |
| Sequence 1959 | AC35218 |
| Sequence 1960 | AC36180 |
| Sequence 1961 | AC36187 |
| Sequence 1962 | AC36079 |
| Sequence 1963 | AC36823 |
| Sequence 1964 | AC36709 |
| Sequence 1965 | AA304651 |
| Sequence 1966 | AA308091 |
| Sequence 1967 | AA442287 |
| Sequence 1968 | AA460440 |
| Sequence 1969 | AA513597 |
| Sequence 1970 | AA576425 |
| Sequence 1971 | AB032983 |
| Sequence 1972 | AF047020 |
| Sequence 1973 | AF103907 |
| Sequence 1974 | AI080439 |
| Sequence 1975 | AI088633 |
| Sequence 1976 | AI267321 |
| Sequence 1977 | AI274700 |
| Sequence 1978 | AI557336 |
| Sequence 1979 | AI825988 |
| Sequence 1980 | AL037105 |
| Sequence 1981 | AW138181 |
| Sequence 1982 | D14043 |
| Sequence 1983 | L38961 |
| Sequence 1984 | M26880 |
| Sequence 1985 | X77548 |
| Sequence 1986 | AB001636 |
| Sequence 1987 | AB008681 |
| Sequence 1988 | AB020866 |
| Sequence 1989 | AC000399 |
| Sequence 1990 | AC002086 |
| Sequence 1991 | AC003991 |
| Sequence 1992 | AC004507 |
| Sequence 1993 | AC004816 |
| Sequence 1994 | AC005383 |
| Sequence 1995 | AC005480 |
| Sequence 1996 | AC005538 |
| Sequence 1997 | AC005831 |
| Sequence 1998 | AC006153 |
| Sequence 1999 | AL031777 |
| Sequence 2000 | AL117340 |
| Sequence 2001 | AL12160 |
| Sequence 2002 | AL121654 |
| Sequence 2003 | AL122007 |
| Sequence 2004 | AL135879 |
| Sequence 2005 | AP000303 |
| Sequence 2006 | M11548 |
| Sequence 2007 | S65371 |
| Sequence 2008 | U13800 |
| Sequence 2009 | U91321 |
| Sequence 2010 | Z63584 |
| Sequence 2011 | Z73900 |
| Sequence 2012 | AA079188 |
| Sequence 2013 | AI267838 |
| Sequence 2014 | AA507791 |
| Sequence 2015 | AA640474 |
| Sequence 2016 | AI023483 |
| Sequence 2017 | AI201484 |
| Sequence 2018 | AA325789 |
| Sequence 2019 | AI151458 |
| Sequence 2020 | AA393870 |
| Sequence 2021 | H70269 |
| Sequence 2022 | AA226321 |
| Sequence 2023 | W58683 |
| Sequence 2024 | AL043205 |
| Sequence 2025 | AA056482 |
| Sequence 2026 | H59731 |
| Sequence 2027 | R69940 |
| Sequence 2028 | AA663192 |
| Sequence 2029 | M449889 |
| Sequence 2030 | AA209369 |
| Sequence 2031 | AA354730 |
| Sequence 2032 | AA354196 |
| Sequence 2033 | AI791370 |
| Sequence 2034 | AA447433 |
| Sequence 2035 | N44743 |
| Sequence 2036 | AA528104 |
| Sequence 2037 | AA853882 |
| Sequence 2038 | AA134726 |
| Sequence 2039 | AA811333 |
| Sequence 2040 | AL121340 |
| Sequence 2041 | AA310201 |
| Sequence 2042 | AI694572 |
| Sequence 2043 | AI140921 |
| Sequence 2044 | AA149579 |
| Sequence 2045 | AA506729 |
| Sequence 2046 | AA877460 |
| Sequence 2047 | AI199094 |
| Sequence 2048 | AA975275 |
| Sequence 2049 | N25122 |
| Sequence 2050 | AI636911 |
| Sequence 2051 | AI219803 |
| Sequence 2052 | AA483044 |
| Sequence 2053 | AA522552 |
| Sequence 2054 | AI564822 |
| Sequence 2055 | R63251 |
| Sequence 2056 | AA526369 |
| Sequence 2057 | AI074652 |
| Sequence 2058 | AA603861 |
| Sequence 2059 | AA253339 |
| Sequence 2060 | AA313170 |
| Sequence 2061 | AA228940 |
| Sequence 2062 | AF001542 |
| Sequence 2063 | AI090642 |
| Sequence 2064 | AA035559 |
| Sequence 2065 | AA304845 |
| Sequence 2066 | AI652112 |
| Sequence 2067 | AA058381 |
| Sequence 2068 | AA935526 |
| Sequence 2069 | AA588023 |
| Sequence 2070 | AI034131 |
| Sequence 2071 | AA577463 |
| Sequence 2072 | AW151704 |
| Sequence 2073 | AI267285 |
| Sequence 2074 | AA531386 |
| Sequence 2075 | AA493331 |
| Sequence 2076 | AL043046 |
| Sequence 2077 | AI373120 |
| Sequence 2078 | AI017239 |
| Sequence 2079 | AA149337 |
| Sequence 2080 | AA622482 |
| Sequence 2081 | AA232147 |
| Sequence 2082 | AI453181 |
| Sequence 2083 | AL121262 |
| Sequence 2084 | AA306322 |
| Sequence 2085 | AI635209 |
| Sequence 2086 | AA973620 |
| Sequence 2087 | AA323181 |
| Sequence 2088 | AA399177 |
| Sequence 2089 | R33609 |
| Sequence 2090 | AW149706 |
| Sequence 2091 | AA878395 |
| Sequence 2092 | AI198859 |
| Sequence 2093 | AA099399 |
| Sequence 2094 | AA467869 |
| Sequence 2095 | AA747909 |
| Sequence 2096 | AA483008 |
| Sequence 2097 | N51806 |
| Sequence 2098 | AA548764 |
| Sequence 2099 | AA229495 |
| Sequence 2100 | AL046322 |
| Sequence 2101 | AA146981 |
| Sequence 2102 | AA152337 |
| Sequence 2103 | AA280917 |
| Sequence 2104 | AL043257 |
| Sequence 2105 | AA026149 |
| Sequence 2106 | AA732538 |
| Sequence 2107 | AA876439 |
| Sequence 2108 | AA405535 |
| Sequence 2109 | H80936 |
| Sequence 2110 | AI378290 |
| Sequence 2111 | AI761098 |
| Sequence 2112 | AA188875 |
| Sequence 2113 | AA703396 |
| Sequence 2114 | AA312076 |
| Sequence 2115 | AA424428 |
| Sequence 2116 | T68102 |
| Sequence 2117 | AA011621 |
| Sequence 2118 | AA285038 |
| Sequence 2119 | AA506459 |
| Sequence 2120 | AA523252 |
| Sequence 2121 | AA522850 |
| Sequence 2122 | AAI59272 |
| Sequence 2123 | AA195010 |
| Sequence 2124 | N72922 |
| Sequence 2125 | AW073731 |
| Sequence 2126 | H25806 |
| Sequence 2127 | D55192 |
| Sequence 2128 | N52271 |
| Sequence 2129 | AA046581 |
| Sequence 2130 | AA579026 |
| Sequence 2131 | AA764981 |
| Sequence 2132 | AI269205 |
| Sequence 2133 | AI754013 |
| Sequence 2134 | AA336412 |
| Sequence 2135 | AW148750 |
| Sequence 2136 | AA304846 |
| Sequence 2137 | AA190998 |
| Sequence 2138 | AA564505 |
| Sequence 2139 | AI086059 |
| Sequence 2140 | AA235224 |
| Sequence 2141 | AA405663 |
| Sequence 2142 | C21370 |
| Sequence 2143 | AI253335 |
| Sequence 2144 | AA437224 |
| Sequence 2145 | AA314893 |
| Sequence 2146 | AI630616 |
| Sequence 2147 | W27229 |
| Sequence 2148 | AA195525 |
| Sequence 2149 | AI868421 |
| Sequence 2150 | AA305969 |
| Sequence 2151 | AI476310 |
| Sequence 2152 | AA843845 |
| Sequence 2153 | AI204916 |
| Sequence 2154 | AA172043 |
| Sequence 2155 | AI557235 |
| Sequence 2156 | AA020899 |
| Sequence 2157 | AI954167 |
| Sequence 2158 | AI380932 |
| Sequence 2159 | AI267502 |
| Sequence 2160 | H30141 |
| Sequence 2161 | AA634408 |
| Sequence 2162 | AA031287 |
| Sequence 2163 | AA233109 |
| Sequence 2164 | AA150421 |
| Sequence 2165 | AA088714 |
| Sequence 2166 | AA523354 |
| Sequence 2167 | AI809963 |
| Sequence 2168 | AA130302 |
| Sequence 2169 | AI831502 |
| Sequence 2170 | AA742522 |
| Sequence 2172 | AA557260 |
| Sequence 2173 | AA291260 |
| Sequence 2174 | M376424 |
| Sequence 2175 | AA807370 |
| Sequence 2176 | H48050 |
| Sequence 2177 | AA320603 |
| Sequence 2178 | AI125653 |
| Sequence 2179 | AA058899 |
| Sequence 2180 | AA016281 |
| Sequence 2181 | AL038234 |
| Sequence 2182 | H08621 |
| Sequence 2183 | AA703816 |
| Sequence 2184 | AA487754 |
| Sequence 2185 | AI217694 |
| Sequence 2186 | AI671394 |
| Sequence 2187 | AA516531 |
| Sequence 2188 | AA322901 |
| Sequence 2189 | AA188140 |
| Sequence 2190 | AI689722 |
| Sequence 2191 | AA599324 |
| Sequence 2192 | H08959 |
| Sequence 2193 | AA308316 |
| Sequence 2194 | N23606 |
| Sequence 2195 | AI620796 |
| Sequence 2196 | AL043048 |
| Sequence 2197 | H02308 |
| Sequence 2198 | AI093387 |
| Sequence 2199 | AA186586 |
| Sequence 2200 | AI697815 |
| Sequence 2201 | AA130271 |
| Sequence 2202 | AA678072 |
| Sequence 2203 | AA468411 |
| Sequence 2204 | AW177204 |
| Sequence 2205 | AL045638 |
| Sequence 2206 | N21237 |
| Sequence 2207 | AA157008 |
| Sequence 2208 | AA150672 |
| Sequence 2209 | C19105 |
| Sequence 2210 | AI066442 |
| Sequence 2211 | AA091998 |
| Sequence 2212 | AI267522 |
| Sequence 2213 | AA071167 |
| Sequence 2214 | AI269747 |
| Sequence 2215 | AA453345 |
| Sequence 2216 | AA876668 |
| Sequence 2217 | AA454208 |
| Sequence 2218 | AA857069 |
| Sequence 2219 | AA765712 |
| Sequence 2220 | AA280256 |
| Sequence 2221 | AA361640 |
| Sequence 2222 | AA256403 |
| Sequence 2223 | AA039967 |
| Sequence 2224 | AA476586 |
| Sequence 2225 | AA147100 |
| Sequence 2226 | AI267612 |
| Sequence 2227 | AA652925 |
| Sequence 2228 | AI564506 |
| Sequence 2229 | AI682937 |
| Sequence 2230 | AA040135 |
| Sequence 2231 | AA114853 |
| Sequence 2232 | AI971281 |
| Sequence 2233 | AA534246 |
| Sequence 2234 | AA205647 |
| Sequence 2235 | AA351504 |
| Sequence 2236 | AA531227 |
| Sequence 2237 | AA402408 |
| Sequence 2238 | AA132904 |
| Sequence 2239 | AI310138 |
| Sequence 2240 | AI267216 |
| Sequence 2241 | AA180941 |
| Sequence 2242 | AL046640 |
| Sequence 2243 | AA504490 |
| Sequence 2244 | AA150220 |
| Sequence 2245 | AA047021 |
| Sequence 2246 | AA450051 |
| Sequence 2247 | H78478 |
| Sequence 2248 | AA225115 |
| Sequence 2249 | C04910 |
| Sequence 2250 | AI114467 |
| Sequence 2251 | N56690 |
| Sequence 2252 | AA992882 |
| Sequence 2253 | AA173184 |
| Sequence 2254 | AA306787 |
| Sequence 2255 | AW157303 |
| Sequence 2256 | AA576425 |
| Sequence 2257 | AA634120 |
| Sequence 2258 | AA675892 |
| Sequence 2259 | AA056667 |
| Sequence 2260 | AI087300 |
| Sequence 2261 | AW170355 |
| Sequence 2262 | N92755 |
| Sequence 2263 | AA009531 |
| Sequence 2264 | AA830807 |
| Sequence 2265 | AA081082 |
| Sequence 2266 | AA598531 |
| Sequence 2267 | W84471 |
| Sequence 2268 | AI053597 |
| Sequence 2269 | AA002222 |
| Sequence 2270 | AA128591 |
| Sequence 2271 | AW028937 |
| Sequence 2272 | AA503943 |
| Sequence 2273 | AA664480 |
| Sequence 2274 | AA101155 |
| Sequence 2275 | AA843176 |
| Sequence 2276 | AI216969 |
| Sequence 2277 | AA313369 |
| Sequence 2278 | AI217172 |
| Sequence 2279 | AA312669 |
| Sequence 2280 | AA501739 |
| Sequence 2281 | AA151012 |
| Sequence 2282 | AA994993 |
| Sequence 2283 | AI133247 |
| Sequence 2284 | AA187576 |
| Sequence 2285 | AA329562 |
| Sequence 2286 | AA442517 |
| Sequence 2287 | AL044723 |
| Sequence 2288 | AA188591 |
| Sequence 2289 | AW176649 |
| Sequence 2290 | AA286678 |
| Sequence 2291 | H96926 |
| Sequence 2292 | AA426229 |
| Sequence 2293 | AA527805 |
| Sequence 2294 | AL040354 |
| Sequence 2295 | AA314146 |
| Sequence 2296 | AA190408 |
| Sequence 2297 | AA457720 |
| Sequence 2298 | AA837457 |
| Sequence 2299 | AI400752 |
| Sequence 2300 | AA143012 |
| Sequence 2301 | AA541528 |
| Sequence 2302 | AA610052 |
| Sequence 2303 | AA128348 |
| Sequence 2304 | AA309749 |
| Sequence 2305 | AI479009 |
| Sequence 2306 | R35797 |
| Sequence 2307 | AA044791 |
| Sequence 2308 | AI984636 |
| Sequence 2309 | R70639 |
| Sequence 2310 | AA480551 |
| Sequence 2311 | AW131127 |
| Sequence 2312 | AA477683 |
| Sequence 2313 | AA366269 |
| Sequence 2314 | AI480345 |
| Sequence 2315 | AA476487 |
| Sequence 2316 | AA037574 |
| Sequence 2317 | AI267254 |
| Sequence 2318 | AA336621 |
| Sequence 2319 | AI659354 |
| Sequence 2320 | AA114078 |
| Sequence 2321 | AA371964 |
| Sequence 2322 | AA551464 |
| Sequence 2323 | AA455475 |
| Sequence 2324 | H61016 |
| Sequence 2325 | AA121625 |
| Sequence 2326 | AA206356 |
| Sequence 2327 | AA932569 |
| Sequence 2328 | AA352179 |
| Sequence 2329 | R15870 |
| Sequence 2330 | AA223320 |
| Sequence 2331 | AA714022 |
| Sequence 2332 | AA648989 |
| Sequence 2333 | AA166952 |
| Sequence 2334 | AI923627 |
| Sequence 2335 | AA315416 |
| Sequence 2336 | AA130890 |
| Sequence 2337 | AI253379 |
| Sequence 2338 | AA584428 |
| Sequence 2339 | AI147850 |
| Sequence 2340 | AA465509 |
| Sequence 2341 | AA150361 |
| Sequence 2342 | AA449940 |
| Sequence 2343 | W26008 |
| Sequence 2344 | AA194162 |
| Sequence 2345 | AA244003 |
| Sequence 2346 | AF150208 |
| Sequence 2347 | AA701467 |
| Sequence 2348 | AA128387 |
| Sequence 2349 | AA226171 |
| Sequence 2350 | AA215927 |
| Sequence 2351 | AA535837 |
| Sequence 2352 | N23010 |
| Sequence 2353 | AA205870 |
| Sequence 2354 | R72403 |
| Sequence 2355 | AI71998 |
| Sequence 2356 | AA252943 |
| Sequence 2357 | AL047776 |
| Sequence 2358 | AA190873 |
| Sequence 2359 | AA838748 |
| Sequence 2360 | AA316110 |
| Sequence 2361 | AA281132 |
| Sequence 2362 | AA310609 |
| Sequence 2363 | AA082936 |
| Sequence 2364 | AA354742 |
| Sequence 2365 | AI697599 |
| Sequence 2366 | AA927433 |
| Sequence 2367 | AI557231 |
| Sequence 2368 | AL039814 |
| Sequence 2369 | AA984384 |
| Sequence 2370 | AA043865 |
| Sequence 2371 | AA491866 |
| Sequence 2372 | AA411736 |
| Sequence 2373 | AA083370 |
| Sequence 2374 | AA863393 |
| Sequence 2375 | AA994857 |
| Sequence 2376 | AI741633 |
| Sequence 2377 | AI077641 |
| Sequence 2378 | AI720603 |
| Sequence 2379 | AA488633 |
| Sequence 2380 | AA780695 |
| Sequence 2381 | AA683080 |
| Sequence 2382 | AW023372 |
| Sequence 2383 | R88918 |
| Sequence 2384 | H11678 |
| Sequence 2385 | AA127220 |
| Sequence 2386 | AA261889 |
| Sequence 2387 | AA699442 |
| Sequence 2388 | AA713504 |
| Sequence 2389 | AA658393 |
| Sequence 2390 | AI99681 |
| Sequence 2391 | H20388 |
| Sequence 2392 | AI375141 |
| Sequence 2393 | N63784 |
| Sequence 2394 | AA193086 |
| Sequence 2395 | AA180137 |
| Sequence 2396 | AI458673 |
| Sequence 2397 | AA101742 |
| Sequence 2398 | AI028097 |
| Sequence 2399 | AA865185 |
| Sequence 2400 | AA071199 |
| Sequence 2401 | AA479962 |
| Sequence 2402 | M133652 |
| Sequence 2403 | AA916145 |
| Sequence 2404 | AA167677 |
| Sequence 2405 | AA904265 |
| Sequence 2406 | AL040264 |
| Sequence 2407 | AA243143 |
| Sequence 2408 | AI093458 |
| Sequence 2409 | AA045381 |
| Sequence 2410 | AA736613 |
| Sequence 2411 | W04240 |
| Sequence 2412 | AI632534 |
| Sequence 2413 | AA651712 |
| Sequence 2414 | AA748064 |
| Sequence 2415 | AA554358 |
| Sequence 2416 | AA229199 |
| Sequence 2417 | AA001805 |
| Sequence 2418 | AA040437 |
| Sequence 2419 | AA340635 |
| Sequence 2420 | AI911776 |
| Sequence 2421 | AA366876 |
| Sequence 2422 | AA806448 |
| Sequence 2423 | AA468167 |
| Sequence 2424 | AA233791 |
| Sequence 2425 | AA315582 |
| Sequence 2426 | AA400296 |
| Sequence 2427 | AA526097 |
| Sequence 2428 | AI623804 |
| Sequence 2429 | AA629033 |
| Sequence 2430 | AI142083 |
| Sequence 2431 | AA156239 |
| Sequence 2432 | AA134016 |
| Sequence 2433 | AA814500 |
| Sequence 2434 | AA041239 |
| Sequence 2435 | AI148251 |
| Sequence 2436 | AA258904 |
| Sequence 2437 | AA315943 |
| Sequence 2438 | AI654183 |
| Sequence 2439 | AA890032 |
| Sequence 2440 | AL118582 |
| Sequence 2441 | AA127136 |
| Sequence 2442 | AI267554 |
| Sequence 2443 | AA457041 |
| Sequence 2444 | AI970919 |
| Sequence 2445 | AL039208 |
| Sequence 2446 | AI557336 |
| Sequence 2447 | AA459455 |
| Sequence 2448 | AA476360 |
| Sequence 2449 | AI080272 |
| Sequence 2450 | AI025262 |
| Sequence 2451 | H03785 |
| Sequence 2452 | AA305627 |
| Sequence 2453 | AA082342 |
| Sequence 2454 | AA186755 |
| Sequence 2455 | AA086128 |
| Sequence 2456 | AI682362 |
| Sequence 2457 | AA280952 |
| Sequence 2458 | AA225294 |
| Sequence 2459 | AI634740 |
| Sequence 2460 | AA364913 |
| Sequence 2461 | AA226266 |
| Sequence 2462 | AA287720 |
| Sequence 2463 | AA883914 |
| Sequence 2464 | AA508861 |
| Sequence 2465 | AA436573 |
| Sequence 2466 | AA937212 |
| Sequence 2467 | AA639174 |
| Sequence 2468 | AI878918 |
| Sequence 2469 | AA173522 |
| Sequence 2470 | AA469129 |
| Sequence 2471 | AA320034 |
| Sequence 2472 | AI755085 |
| Sequence 2473 | AI242701 |
| Sequence 2474 | AA431018 |
| Sequence 2475 | AA478539 |
| Sequence 2476 | AA531255 |
| Sequence 2477 | AI080480 |
| Sequence 2478 | AA669300 |
| Sequence 2479 | AA476725 |
| Sequence 2480 | AI557626 |
| Sequence 2481 | AA477728 |
| Sequence 2482 | AW157674 |
| Sequence 2483 | AA592908 |
| Sequence 2484 | AA932402 |
| Sequence 2485 | AA308051 |
| Sequence 2486 | AI686325 |
| Sequence 2487 | AI951118 |
| Sequence 2488 | AI768568 |
| Sequence 2489 | AL041012 |
| Sequence 2490 | AA557683 |
| Sequence 2491 | AI207528 |
| Sequence 2492 | AA133276 |
| Sequence 2493 | AA167366 |
| Sequence 2494 | AI923011 |
| Sequence 2495 | AL048954 |
| Sequence 2496 | AW003952 |
| Sequence 2497 | AA988520 |
| Sequence 2498 | AA995613 |
| Sequence 2499 | AL047056 |
| Sequence 2500 | AA187710 |
| Sequence 2501 | AI636628 |
| Sequence 2502 | AA315805 |
| Sequence 2503 | AI570859 |
| Sequence 2504 | AA425908 |
| Sequence 2505 | AA765947 |
| Sequence 2506 | F13179 |
| Sequence 2507 | AA534505 |
| Sequence 2508 | AA375164 |
| Sequence 2509 | AA205996 |
| Sequence 2510 | AL118746 |
| Sequence 2511 | AA652600 |
| Sequence 2512 | AA568488 |
| Sequence 2513 | AA994966 |
| Sequence 2514 | AA443447 |
| Sequence 2515 | AA165205 |
| Sequence 2516 | AA070250 |
| Sequence 2517 | AI961028 |
| Sequence 2518 | AI200245 |
| Sequence 2519 | A1801373 |
| Sequence 2520 | AA336387 |
| Sequence 2521 | AA740911 |
| Sequence 2522 | H20744 |
| Sequence 2523 | AA316423 |
| Sequence 2524 | AA094656 |
| Sequence 2525 | AA902953 |
| Sequence 2526 | AA532852 |
| Sequence 2527 | AA224487 |
| Sequence 2528 | AL035985 |
| Sequence 2529 | AI738488 |
| Sequence 2530 | AA335563 |
| Sequence 2531 | AA580417 |
| Sequence 2532 | AA425062 |
| Sequence 2533 | AA464941 |
| Sequence 2534 | AA305143 |
| Sequence 2535 | AA402721 |
| Sequence 2536 | AI620924 |
| Sequence 2537 | AA026343 |
| Sequence 2538 | AA156690 |
| Sequence 2539 | AA102570 |
| Sequence 2540 | R70675 |
| Sequence 2541 | AA503719 |
| Sequence 2542 | AA021513 |
| Sequence 2543 | AI458521 |
| Sequence 2544 | AL037053 |
| Sequence 2545 | H24356 |
| Sequence 2546 | AW105540 |
| Sequence 2547 | AA255974 |
| Sequence 2548 | AA545726 |
| Sequence 2549 | AA993438 |
| Sequence 2550 | AA040439 |
| Sequence 2551 | AA176847 |
| Sequence 2552 | H16224 |
| Sequence 2553 | AA410965 |
| Sequence 2554 | AI005274 |
| Sequence 2555 | A1129280 |
| Sequence 2556 | AA101224 |
| Sequence 2557 | AI244140 |
| Sequence 2558 | AI798840 |
| Sequence 2559 | AL079982 |
| Sequence 2560 | AA316181 |
| Sequence 2561 | AI267570 |
| Sequence 2562 | AA779602 |
| Sequence 2563 | AA577585 |
| Sequence 2564 | AA205949 |
| Sequence 2565 | AI265881 |
| Sequence 2566 | W87865 |
| Sequence 2567 | H13406 |
| Sequence 2568 | AA766050 |
| Sequence 2569 | AA610444 |
| Sequence 2570 | AA447343 |
| Sequence 2571 | AA293873 |
| Sequence 2572 | AA490910 |
| Sequence 2573 | AA742189 |
| Sequence 2574 | AA158507 |
| Sequence 2575 | AA370116 |
| Sequence 2576 | AAO81712 |
| Sequence 2577 | AA357574 |
| Sequence 2578 | AA317455 |
| Sequence 2579 | AA455668 |
| Sequence 2580 | AI080439 |
| Sequence 2581 | AI381649 |
| Sequence 2582 | AA225843 |
| Sequence 2583 | AA342832 |
| Sequence 2584 | AA447322 |
| Sequence 2585 | AA505594 |
| Sequence 2586 | AA172195 |
| Sequence 2587 | AA526214 |
| Sequence 2588 | AI474235 |
| Sequence 2589 | AW023413 |
| Sequence 2590 | AA112308 |
| Sequence 2591 | AA476756 |
| Sequence 2592 | AA317040 |
| Sequence 2593 | W05472 |
| Sequence 2594 | AA256591 |
| Sequence 2595 | AA311125 |
| Sequence 2596 | AA884249 |
| Sequence 2597 | AA401003 |
| Sequence 2598 | AA148780 |
| Sequence 2599 | AA155999 |
| Sequence 2600 | AI557599 |
| Sequence 2601 | AI146340 |
| Sequence 2602 | AA431664 |
| Sequence 2603 | AI753951 |
| Sequence 2604 | AA081169 |
| Sequence 2605 | AA326752 |
| Sequence 2606 | H57382 |
| Sequence 2607 | AA385449 |
| Sequence 2608 | AI989530 |
| Sequence 2609 | AA527730 |
| Sequence 2610 | AA632742 |
| Sequence 2611 | AA370507 |
| Sequence 2612 | AA053557 |
| Sequence 2613 | AA527326 |
| Sequence 2614 | AA453609 |
| Sequence 2615 | AA506419 |
| Sequence 2616 | AA262226 |
| Sequence 2617 | H54240 |
| Sequence 2618 | AA741050 |
| Sequence 2619 | AI342269 |
| Sequence 2620 | AA339735 |
| Sequence 2621 | AL038549 |
| Sequence 2622 | AA326165 |
| Sequence 2623 | AA641601 |
| Sequence 2624 | AA599078 |
| Sequence 2625 | AI914133 |
| Sequence 2626 | AA344131 |
| Sequence 2627 | AI088691 |
| Sequence 2628 | AI400511 |
| Sequence 2629 | AA528202 |
| Sequence 2630 | AA148215 |
| Sequence 2631 | AA479976 |
| Sequence 2632 | W61049 |
| Sequence 2633 | AA479860 |
| Sequence 2634 | AI401150 |
| Sequence 2635 | H25880 |
| Sequence 2636 | AL036415 |
| Sequence 2637 | AI693198 |
| Sequence 2638 | AA256420 |
| Sequence 2639 | AA406594 |
| Sequence 2640 | AA594540 |
| Sequence 2641 | AA701667 |
| Sequence 2642 | AA147871 |
| Sequence 2643 | AA192597 |
| Sequence 2644 | AA972792 |
| Sequence 2645 | AA232374 |
| Sequence 2646 | AA234050 |
| Sequence 2647 | AI248721 |
| Sequence 2648 | AA507511 |
| Sequence 2649 | AA503215 |
| Sequence 2650 | H05325 |
| Sequence 2651 | AA453310 |
| Sequence 2652 | AL039293 |
| Sequence 2653 | AA211853 |
| Sequence 2654 | AA531563 |
| Sequence 2655 | AA219705 |
| Sequence 2656 | AA449054 |
| Sequence 2657 | R11581 |
| Sequence 2658 | AI074308 |
| Sequence 2659 | AA278241 |
| Sequence 2660 | AA844734 |
| Sequence 2661 | AI207660 |
| Sequence 2662 | AA352938 |
| Sequence 2663 | AA357069 |
| Sequence 2664 | N32485 |
| Sequence 2665 | AA481158 |
| Sequence 2666 | AI161350 |
| Sequence 2667 | AI867729 |
| Sequence 2668 | AI075631 |
| Sequence 2669 | AA284997 |
| Sequence 2670 | AA394302 |
| Sequence 2671 | AI929611 |
| Sequence 2672 | AI659440 |
| Sequence 2673 | AL037638 |
| Sequence 2674 | AA773998 |
| Sequence 2675 | AA481571 |
| Sequence 2676 | R51737 |
| Sequence 2677 | AA468565 |
| Sequence 2678 | AI823661 |
| Sequence 2679 | AA524672 |
| Sequence 2680 | AI378932 |
| Sequence 2681 | AI458743 |
| Sequence 2682 | T77631 |
| Sequence 2683 | AI052738 |
| Sequence 2684 | AA203119 |
| Sequence 2685 | W27182 |
| Sequence 2686 | AI968159 |
| Sequence 2687 | AA313684 |
| Sequence 2688 | AI129891 |
| Sequence 2689 | R83716 |
| Sequence 2690 | AA359832 |
| Sequence 2691 | R39716 |
| Sequence 2692 | AA120781 |
| Sequence 2693 | AA480844 |
| Sequence 2694 | AA417097 |
| Sequence 2695 | AW086063 |
| Sequence 2696 | AI791906 |
| Sequence 2697 | AA424456 |
| Sequence 2698 | AA121266 |
| Sequence 2699 | AI249680 |
| Sequence 2700 | AA652478 |
| Sequence 2702 | AL079681 |
| Sequence 2703 | AA164369 |
| Sequence 2704 | AL040936 |
| Sequence 2705 | AA569482 |
| Sequence 2706 | AA312968 |
| Sequence 2707 | AA523498 |
| Sequence 2708 | AA032194 |
| Sequence 2709 | AL047436 |
| Sequence 2710 | AA035102 |
| Sequence 2711 | D81635 |
| Sequence 2712 | AL121423 |
| Sequence 2713 | AA099896 |
| Sequence 2714 | AA177054 |
| Sequence 2715 | AA292158 |
| Sequence 2716 | AA224953 |
| Sequence 2717 | AA479444 |
| Sequence 2718 | AI417131 |
| Sequence 2719 | AA115604 |
| Sequence 2720 | R19977 |
| Sequence 2721 | AA464133 |
| Sequence 2722 | AA345189 |
| Sequence 2723 | AA503096 |
| Sequence 2724 | AA021115 |
| Sequence 2725 | AA318745 |
| Sequence 2726 | AA190661 |
| Sequence 2727 | R69724 |
| Sequence 2728 | AI144378 |
| Sequence 2729 | AA524651 |
| Sequence 2730 | AA524528 |
| Sequence 2731 | AW138413 |
| Sequence 2732 | AI627356 |
| Sequence 2733 | AA038823 |
| Sequence 2734 | AI276705 |
| Sequence 2735 | AA946883 |
| Sequence 2736 | AA831078 |
| Sequence 2737 | AA378589 |
| Sequence 2738 | AI565591 |
| Sequence 2739 | AA730111 |
| Sequence 2740 | AA355264 |
| Sequence 2741 | AA084987 |
| Sequence 2742 | AA143034 |
| Sequence 2743 | AA188315 |
| Sequence 2744 | T98099 |
| Sequence 2745 | AA102183 |
| Sequence 2746 | AI570128 |
| Sequence 2747 | AA934530 |
| Sequence 2748 | AA206647 |
| Sequence 2749 | AA534235 |
| Sequence 2750 | AA113823 |
| Sequence 2751 | AA083345 |
| Sequence 2752 | AA421112 |
| Sequence 2753 | AA493551 |
| Sequence 2754 | AI267652 |
| Sequence 2755 | AI085554 |
| Sequence 2756 | AA318422 |
| Sequence 2757 | AA135812 |
| Sequence 2758 | R51540 |
| Sequence 2759 | AA188854 |
| Sequence 2760 | AA227830 |
| Sequence 2761 | AA255717 |
| Sequence 2762 | AA365883 |
| Sequence 2763 | AA860246 |
| Sequence 2764 | AA069078 |
| Sequence 2765 | AI332588 |
| Sequence 2766 | AA446760 |
| Sequence 2767 | AI033912 |
| Sequence 2768 | H03882 |
| Sequence 2769 | AA298464 |
| Sequence 2770 | AA224994 |
| Sequence 2771 | AA379370 |
| Sequence 2772 | AA228273 |
| Sequence 2773 | AA136437 |
| Sequence 2774 | N40598 |
| Sequence 2775 | AA563986 |
| Sequence 2776 | AW022017 |
| Sequence 2777 | AA779728 |
| Sequence 2778 | AA040620 |
| Sequence 2779 | AI808684 |
| Sequence 2780 | AA625145 |
| Sequence 2781 | AA862821 |
| Sequence 2782 | AA468839 |
| Sequence 2783 | AA375135 |
| Sequence 2784 | AW169765 |
| Sequence 2785 | AA659719 |
| Sequence 2786 | AA131961 |
| Sequence 2787 | AA315331 |
| Sequence 2788 | AA404615 |
| Sequence 2789 | AI697873 |
| Sequence 2790 | AA578773 |
| Sequence 2791 | AI859442 |
| Sequence 2792 | AA433880 |
| Sequence 2793 | AI025519 |
| Sequence 2794 | AA481979 |
| Sequence 2795 | AA353655 |
| Sequence 2796 | AA088344 |
| Sequence 2797 | AA196597 |
| Sequence 2798 | AA760894 |
| Sequence 2799 | AA113333 |
| Sequence 2800 | AA479284 |
| Sequence 2801 | W35301 |
| Sequence 2802 | M070008 |
| Sequence 2803 | M833759 |
| Sequence 2804 | AI064763 |
| Sequence 2805 | AA120782 |
| Sequence 2806 | AA129551 |
| Sequence 2807 | AA215777 |
| Sequence 2808 | AA150459 |
| Sequence 2809 | AA436540 |
| Sequence 2810 | AI609659 |
| Sequence 2811 | AA031528 |
| Sequence 2812 | AA446943 |
| Sequence 2813 | AA356069 |
| Sequence 2814 | AI761190 |
| Sequence 2815 | AA040694 |
| Sequence 2816 | AI078802 |
| Sequence 2817 | AI693302 |
| Sequence 2818 | AA513052 |
| Sequence 2819 | AA427459 |
| Sequence 2820 | AA044586 |
| Sequence 2821 | AA379449 |
| Sequence 2822 | AI625404 |
| Sequence 2823 | AA333300 |
| Sequence 2824 | AI253300 |
| Sequence 2825 | AI253424 |
| Sequence 2826 | AI374628 |
| Sequence 2827 | AA069845 |
| Sequence 2828 | N86927 |
| Sequence 2829 | AA469199 |
| Sequence 2830 | AI267416 |
| Sequence 2831 | AA234135 |
| Sequence 2832 | AA286708 |
| Sequence 2833 | AI808505 |
| Sequence 2834 | H78888 |
| Sequence 2835 | T81369 |
| Sequence 2836 | R82456 |
| Sequence 2837 | AA812581 |
| Sequence 2838 | AA565214 |
| Sequence 2839 | AA262508 |
| Sequence 2840 | AA534644 |
| Sequence 2841 | AA152276 |
| Sequence 2842 | T84605 |
| Sequence 2843 | AA523902 |
| Sequence 2844 | AL037812 |
| Sequence 2845 | AI245297 |
| Sequence 2846 | AI198853 |
| Sequence 2847 | AA813266 |
| Sequence 2848 | AI217015 |
| Sequence 2849 | AI557639 |
| Sequence 2850 | AA182513 |
| Sequence 2851 | AA836233 |
| Sequence 2852 | AA557321 |
| Sequence 2853 | AI198311 |
| Sequence 2854 | AA903139 |
| Sequence 2855 | AI627818 |
| Sequence 2856 | AA323813 |
| Sequence 2857 | AI469767 |
| Sequence 2858 | R18975 |
| Sequence 2859 | AA196978 |
| Sequence 2860 | AA809784 |
| Sequence 2861 | AA502798 |
| Sequence 2862 | AA568207 |
| Sequence 2863 | AI524414 |
| Sequence 2864 | AI300489 |
| Sequence 2865 | AA129574 |
| Sequence 2866 | AA577672 |
| Sequence 2867 | AI762634 |
| Sequence 2868 | AA071372 |
| Sequence 2869 | AL036902 |
| Sequence 2870 | AI267162 |
| Sequence 2871 | AA463533 |
| Sequence 2872 | N89861 |
| Sequence 2873 | AA206675 |
| Sequence 2874 | AA418477 |
| Sequence 2875 | AA639972 |
| Sequence 2876 | AI620284 |
| Sequence 2877 | AA515908 |
| Sequence 2878 | AA133742 |
| Sequence 2879 | H49828 |
| Sequence 2880 | AA911245 |
| Sequence 2881 | AA505816 |
| Sequence 2882 | AA121836 |
| Sequence 2883 | R52729 |
| Sequence 2884 | AA161217 |
| Sequence 2885 | AA731537 |
| Sequence 2886 | AI588087 |
| Sequence 2887 | AW152265 |
| Sequence 2888 | AA017292 |
| Sequence 2889 | N24284 |
| Sequence 2890 | AA088606 |
| Sequence 2891 | T66348 |
| Sequence 2892 | AA492280 |
| Sequence 2893 | AI093823 |
| Sequence 2894 | AA836834 |
| Sequence 2895 | AA737421 |
| Sequence 2896 | AA812081 |
| Sequence 2897 | AA960924 |
| Sequence 2898 | W27484 |
| Sequence 2899 | AA362826 |
| Sequence 2900 | AA220921 |
| Sequence 2901 | AI804733 |
| Sequence 2902 | AI754874 |
| Sequence 2903 | AA027126 |
| Sequence 2904 | W63659 |
| Sequence 2905 | AI804327 |
| Sequence 2906 | AA165152 |
| Sequence 2907 | AA112198 |
| Sequence 2908 | AI114489 |
| Sequence 2909 | AW173278 |
| Sequence 2910 | AA296298 |
| Sequence 2911 | AI312130 |
| Sequence 2912 | AA369786 |
| Sequence 2913 | AI394364 |
| Sequence 2914 | AA206004 |
| Sequence 2915 | AI627750 |
| Sequence 2916 | AI340246 |
| Sequence 2917 | AA084197 |
| Sequence 2918 | AI267492 |
| Sequence 2919 | AI207515 |
| Sequence 2920 | AI356967 |
| Sequence 2921 | W58562 |
| Sequence 2922 | AA151796 |
| Sequence 2923 | W27968 |
| Sequence 2924 | AA312591 |
| Sequence 2925 | AI332698 |
| Sequence 2926 | AA654621 |
| Sequence 2927 | AI290658 |
| Sequence 2928 | AI272941 |
| Sequence 2929 | AI815498 |
| Sequence 2930 | H57064 |
| Sequence 2931 | AA262537 |
| Sequence 2932 | AI590878 |
| Sequence 2933 | AA229872 |
| Sequence 2934 | AI815196 |
| Sequence 2935 | AA541515 |
| Sequence 2936 | AA454921 |
| Sequence 2937 | AA164983 |
| Sequence 2938 | AA034255 |
| Sequence 2939 | AA321703 |
| Sequence 2940 | AA564513 |
| Sequence 2941 | AI889759 |
| Sequence 2942 | AA527116 |
| Sequence 2943 | AA470690 |
| Sequence 2944 | AA349118 |
| Sequence 2945 | AI351780 |
| Sequence 2946 | AA026288 |
| Sequence 2947 | AI031665 |
| Sequence 2948 | R89611 |
| Sequence 2949 | AI914231 |
| Sequence 2950 | AA165165 |
| Sequence 2951 | AL039399 |
| Sequence 2952 | AA187383 |
| Sequence 2953 | AI090786 |
| Sequence 2954 | AI659898 |
| Sequence 2955 | R34894 |
| Sequence 2956 | AI075963 |
| Sequence 2957 | AI557112 |
| Sequence 2958 | AA992606 |
| Sequence 2959 | AF034174 |
| Sequence 2960 | H00112 |
| Sequence 2961 | AA972598 |
| Sequence 2962 | N28331 |
| Sequence 2963 | R46295 |
| Sequence 2964 | AA425407 |
| Sequence 2965 | N34141 |
| Sequence 2966 | AI688573 |
| Sequence 2967 | AA227579 |
| Sequence 2968 | AA131834 |
| Sequence 2969 | AA043692 |
| Sequence 2970 | AA575898 |
| Sequence 2971 | AA195259 |
| Sequence 2972 | AA613916 |
| Sequence 2973 | AI815495 |
| Sequence 2974 | AA452200 |
| Sequence 2975 | AW022756 |
| Sequence 2976 | AA489433 |
| Sequence 2977 | Z46212 |
| Sequence 2978 | T99603 |
| Sequence 2979 | AA513056 |
| Sequence 2980 | R39446 |
| Sequence 2981 | AL037583 |
| Sequence 2982 | AI267454 |
| Sequence 2983 | AI307302 |
| Sequence 2984 | AI740900 |
| Sequence 2985 | AA478931 |
| Sequence 2986 | H01539 |
| Sequence 2987 | AI351045 |
| Sequence 2988 | AI478385 |
| Sequence 2989 | AA890010 |
| Sequence 2990 | AA513556 |
| Sequence 2991 | AA429336 |
| Sequence 2992 | AA846576 |
| Sequence 2993 | AA259189 |
| Sequence 2994 | AI948503 |
| Sequence 2995 | AI589075 |
| Sequence 2996 | AA552410 |
| Sequence 2997 | AA923172 |
| Sequence 2998 | AI267461 |
| Sequence 2999 | AA243065 |
| Sequence 3000 | AA150001 |
| Sequence 3001 | AI972279 |
| Sequence 3002 | AA383420 |
| Sequence 3003 | AA492192 |
| Sequence 3004 | AA890206 |
| Sequence 3005 | AA477747 |
| Sequence 3006 | AI628389 |
| Sequence 3007 | AA053510 |
| Sequence 3008 | AA613587 |
| Sequence 3009 | AA399326 |
| Sequence 3010 | AA280406 |
| Sequence 3011 | AA147341 |
| Sequence 3012 | AA314203 |
| Sequence 3013 | AA304947 |
| Sequence 3014 | AA449544 |
| Sequence 3015 | AA583461 |
| Sequence 3016 | AA247738 |
| Sequence 3017 | N46575 |
| Sequence 3018 | AA022453 |
| Sequence 3019 | AA587806 |
| Sequence 3020 | AA334754 |
| Sequence 3021 | AI590351 |
| Sequence 3022 | AW167665 |
| Sequence 3023 | AA573557 |
| Sequence 3024 | AW005971 |
| Sequence 3025 | AI521156 |
| Sequence 3026 | AA367948 |
| Sequence 3027 | AA399265 |
| Sequence 3028 | AI796507 |
| Sequence 3029 | AA081426 |
| Sequence 3030 | AA420845 |
| Sequence 3031 | AA371205 |
| Sequence 3032 | AA120964 |
| Sequence 3033 | AA627939 |
| Sequence 3034 | AL119562 |
| Sequence 3035 | AA349865 |
| Sequence 3036 | AI184994 |
| Sequence 3037 | AA299595 |
| Sequence 3038 | AA640928 |
| Sequence 3039 | AA302480 |
| Sequence 3040 | AA410968 |
| Sequence 3041 | R69818 |
| Sequence 3042 | AA171454 |
| Sequence 3043 | AI815033 |
| Sequence 3044 | AI743852 |
| Sequence 3045 | AI991546 |
| Sequence 3046 | AA099141 |
| Sequence 3047 | W93718 |
| Sequence 3048 | AA253269 |
| Sequence 3049 | AA612666 |
| Sequence 3050 | AA58I956 |
| Sequence 3051 | AA045367 |
| Sequence 3052 | AI082864 |
| Sequence 3053 | AA315460 |
| Sequence 3054 | AA430555 |
| Sequence 3055 | AA573187 |
| Sequence 3056 | AL110395 |
| Sequence 3057 | AA806060 |
| Sequence 3058 | AA314188 |
| Sequence 3059 | AA280054 |
| Sequence 3060 | AA252468 |
| Sequence 3061 | AA311466 |
| Sequence 3062 | AA220966 |
| Sequence 3063 | AA654650 |
| Sequence 3064 | AA459753 |
| Sequence 3065 | AA021037 |
| Sequence 3066 | C15094 |
| Sequence 3067 | AA545727 |
| Sequence 3068 | AA134023 |
| Sequence 3069 | AA657830 |
| Sequence 3070 | AA251242 |
| Sequence 3071 | AA131640 |
| Sequence 3072 | AA572895 |
| Sequence 3073 | AI341700 |
| Sequence 3074 | AA092316 |
| Sequence 3075 | AA577598 |
| Sequence 3076 | AA147911 |
| Sequence 3077 | AA292871 |
| Sequence 3078 | AA442145 |
| Sequence 3079 | AA680052 |
| Sequence 3080 | AA039546 |
| Sequence 3081 | AW067912 |
| Sequence 3082 | AI267185 |
| Sequence 3083 | AA148262 |
| Sequence 3084 | AA461222 |
| Sequence 3085 | AW080160 |
| Sequence 3086 | N52554 |
| Sequence 3087 | AA401784 |
| Sequence 3088 | AA447948 |
| Sequence 3089 | AA046763 |
| Sequence 3090 | AI148392 |
| Sequence 3091 | AA195063 |
| Sequence 3092 | AA410580 |
| Sequence 3093 | AI127923 |
| Sequence 3094 | AA644237 |
| Sequence 3095 | AA631868 |
| Sequence 3096 | AI910526 |
| Sequence 3097 | AA197003 |
| Sequence 3098 | AW021628 |
| Sequence 3099 | AL036483 |
| Sequence 3100 | AI557225 |
| Sequence 3101 | AA420524 |
| Sequence 3102 | AA452120 |
| Sequence 3103 | AA452027 |
| Sequence 3104 | AI962319 |
| Sequence 3105 | AA204703 |
| Sequence 3106 | AA219224 |
| Sequence 3107 | AA035012 |
| Sequence 3108 | AA496801 |
| Sequence 3109 | AA604497 |
| Sequence 3110 | AI267845 |
| Sequence 3111 | AA485767 |
| Sequence 3112 | AA452920 |
| Sequence 3113 | AA470051 |
| Sequence 3114 | R77185 |
| Sequence 3115 | AA150309 |
| Sequence 3116 | R55543 |
| Sequence 3117 | AI366374 |
| Sequence 3118 | AA029673 |
| Sequence 3119 | AA826649 |
| Sequence 3120 | AA299140 |
| Sequence 3121 | AI198986 |
| Sequence 3122 | N36967 |
| Sequence 3123 | AA400418 |
| Sequence 3124 | AA233409 |
| Sequence 3125 | AA069692 |
| Sequence 3126 | AA618601 |
| Sequence 3127 | AA043594 |
| Sequence 3128 | AI817249 |
| Sequence 3129 | AI065140 |
| Sequence 3130 | AI061116 |
| Sequence 3131 | AI685124 |
| Sequence 3132 | N36090 |
| Sequence 3133 | AA234318 |
| Sequence 3134 | AA203492 |
| Sequence 3135 | N73033 |
| Sequence 3136 | AA931164 |
| Sequence 3137 | AI694767 |
| Sequence 3138 | AA280221 |
| Sequence 3139 | AA325077 |
| Sequence 3140 | AA316159 |
| Sequence 3141 | AA017189 |
| Sequence 3142 | AA782483 |
| Sequence 3143 | AI216138 |
| Sequence 3144 | AA683136 |
| Sequence 3145 | AA366415 |
| Sequence 3146 | AA131327 |
| Sequence 3147 | AA348734 |
| Sequence 3148 | AL045447 |
| Sequence 3149 | AL049935 |
| Sequence 3150 | AF073298 |
| Sequence 3151 | S39329 |
| Sequence 3152 | Y12860 |
| Sequence 3153 | AF103720 |
| Sequence 3154 | AF071202 |
| Sequence 3155 | AF026166 |
| Sequence 3156 | M36634 |
| Sequence 3157 | AF058953 |
| Sequence 3158 | AF070525 |
| Sequence 3159 | AB014601 |
| Sequence 3160 | AF019214 |
| Sequence 3161 | AF176574 |
| Sequence 3162 | M63180 |
| Sequence 3163 | AB000468 |
| Sequence 3164 | M16768 |
| Sequence 3165 | AB007956 |
| Sequence 3166 | X73608 |
| Sequence 3167 | M17323 |
| Sequence 3168 | AL117644 |
| Sequence 3169 | D10495 |
| Sequence 3170 | M21895 |
| Sequence 3171 | M83822 |
| Sequence 3172 | J02642 |
| Sequence 3173 | AB002369 |
| Sequence 3174 | AB007972 |
| Sequence 3175 | AF151837 |
| Sequence 3176 | U54558 |
| Sequence 3177 | AB007957 |
| Sequence 3178 | M19961 |
| Sequence 3179 | AF085845 |
| Sequence 3180 | X94910 |
| Sequence 3181 | AJ133005 |
| Sequence 3182 | AL050265 |
| Sequence 3183 | AB018319 |
| Sequence 3184 | M86752 |
| Sequence 3185 | X53793 |
| Sequence 3186 | AB022663 |
| Sequence 3187 | AF065388 |
| Sequence 3188 | S73498 |
| Sequence 3189 | AL050091 |
| Sequence 3190 | U40282 |
| Sequence 3191 | X78136 |
| Sequence 3192 | M16660 |
| Sequence 3193 | AF016509 |
| Sequence 3194 | AF151840 |
| Sequence 3195 | AF070618 |
| Sequence 3196 | AF057160 |
| Sequence 3197 | X69086 |
| Sequence 3198 | X81836 |
| Sequence 3199 | U09820 |
| Sequence 3200 | AF068302 |
| Sequence 3201 | AB006965 |
| Sequence 3202 | AF045001 |
| Sequence 3203 | U07991 |
| Sequence 3204 | AB011145 |
| Sequence 3205 | AF144755 |
| Sequence 3206 | M55543 |
| Sequence 3207 | AB033017 |
| Sequence 3208 | AF052097 |
| Sequence 3209 | Y11435 |
| Sequence 3210 | AJ131753 |
| Sequence 3211 | X66276 |
| Sequence 3212 | AB014563 |
| Sequence 3213 | AF093097 |
| Sequence 3214 | AJ012499 |
| Sequence 3215 | AF007151 |
| Sequence 3216 | M93651 |
| Sequence 3217 | AB018358 |
| Sequence 3218 | Y00345 |
| Sequence 3219 | AF109219 |
| Sequence 3220 | U12465 |
| Sequence 3221 | L09235 |
| Sequence 3222 | AB005289 |
| Sequence 3223 | D21260 |
| Sequence 3224 | L05425 |
| Sequence 3225 | X68560 |
| Sequence 3226 | M73547 |
| Sequence 3227 | D63486 |
| Sequence 3228 | J04205 |
| Sequence 3229 | X80910 |
| Sequence 3230 | X05908 |
| Sequence 3231 | E02822 |
| Sequence 3232 | AF054175 |
| Sequence 3233 | AF016369 |
| Sequence 3234 | X56351 |
| Sequence 3235 | AF103907 |
| Sequence 3236 | D28480 |
| Sequence 3237 | AF034607 |
| Sequence 3238 | AJ010842 |
| Sequence 3239 | AB011159 |
| Sequence 3240 | AB006621 |
| Sequence 3241 | U09813 |
| Sequence 3242 | AB001636 |
| Sequence 3243 | AF149045 |
| Sequence 3244 | AF055994 |
| Sequence 3245 | M26663 |
| Sequence 3246 | AB032983 |
| Sequence 3247 | U80456 |
| Sequence 3248 | AF087438 |
| Sequence 3249 | AB029027 |
| Sequence 3250 | AF096773 |
| Sequence 3251 | U12596 |
| Sequence 3252 | D80009 |
| Sequence 3253 | AF078863 |
| Sequence 3254 | M77804 |
| Sequence 3255 | M24902 |
| Sequence 3256 | M22590 |
| Sequence 3257 | AF077200 |
| Sequence 3258 | AF125100 |
| Sequence 3259 | AF041259 |
| Sequence 3260 | L27841 |
| Sequence 3261 | AF032922 |
| Sequence 3262 | U41806 |
| Sequence 3263 | AL049219 |
| Sequence 3264 | AB002451 |
| Sequence 3265 | D14696 |
| Sequence 3266 | X06537 |
| Sequence 3267 | E05692 |
| Sequence 3268 | M29366 |
| Sequence 3269 | AB007947 |
| Sequence 3270 | X97065 |
| Sequence 3271 | U37518 |
| Sequence 3272 | AB032959 |
| Sequence 3273 | X70326 |
| Sequence 3274 | AB033051 |
| Sequence 3275 | M10905 |
| Sequence 3276 | AF070553 |
| Sequence 3277 | AF032885 |
| Sequence 3278 | J04611 |
| Sequence 3279 | S75755 |
| Sequence 3280 | D84476 |
| Sequence 3281 | J04443 |
| Sequence 3282 | AL050192 |
| Sequence 3283 | AF131856 |
| Sequence 3284 | AB014531 |
| Sequence 3285 | AB019524 |
| Sequence 3286 | M64571 |
| Sequence 3287 | X60111 |
| Sequence 3288 | AL110214 |
| Sequence 3289 | D43947 |
| Sequence 3290 | AF000983 |
| Sequence 3291 | AF124438 |
| Sequence 3292 | AB007510 |
| Sequence 3293 | AF000994 |
| Sequence 3294 | AJ004913 |
| Sequence 3295 | AF055470 |
| Sequence 3296 | AJ005016 |
| Sequence 3297 | X57398 |
| Sequence 3298 | M20681 |
| Sequence 3299 | U15008 |
| Sequence 3300 | S70290 |
| Sequence 3301 | AB032991 |
| Sequence 3302 | AF097514 |
| Sequence 3303 | S78203 |
| Sequence 3304 | AF010233 |
| Sequence 3305 | AF070660 |
| Sequence 3306 | AF000982 |
| Sequence 3307 | U79716 |
| Sequence 3308 | Z22548 |
| Sequence 3309 | AF084260 |
| Sequence 3310 | AF151859 |
| Sequence 3311 | AL050373 |
| Sequence 3312 | Z26317 |
| Sequence 3313 | U46689 |
| Sequence 3314 | M94556 |
| Sequence 3315 | S67859 |
| Sequence 3316 | AB023153 |
| Sequence 3317 | AF021351 |
| Sequence 3318 | E02628 |
| Sequence 3319 | AL110179 |
| Sequence 3320 | AB028945 |
| Sequence 3321 | AF054179 |
| Sequence 3322 | U09848 |
| Sequence 3323 | U43899 |
| Sequence 3324 | AJ249248 |
| Sequence 3325 | L25899 |
| Sequence 3326 | U13616 |
| Sequence 3327 | AB032948 |
| Sequence 3328 | L15702 |
| Sequence 3329 | AF017748 |
| Sequence 3330 | U14528 |
| Sequence 3331 | AF098533 |
| Sequence 3332 | L07033 |
| Sequence 3333 | M14648 |
| Sequence 3334 | X56932 |
| Sequence 3335 | AB001106 |
| Sequence 3336 | J05533 |
| Sequence 3337 | AF114817 |
| Sequence 3338 | AF150105 |
| Sequence 3339 | D13315 |
| Sequence 3340 | AI049276 |
| Sequence 3341 | AF153608 |
| Sequence 3342 | D13629 |
| Sequence 3343 | AF188745 |
| Sequence 3344 | AF198358 |
| Sequence 3345 | D38305 |
| Sequence 3346 | AF007791 |
| Sequence 3347 | AF100756 |
| Sequence 3348 | AB008430 |
| Sequence 3349 | D87666 |
| Sequence 3350 | L12168 |
| Sequence 3351 | E08764 |
| Sequence 3352 | M69106 |
| Sequence 3353 | AF012281 |
| Sequence 3354 | E00195 |
| Sequence 3355 | L15203 |
| Sequence 3356 | U39318 |
| Sequence 3357 | AB033078 |
| Sequence 3358 | M64241 |
| Sequence 3359 | AF147331 |
| Sequence 3360 | AF070561 |
| Sequence 3361 | Z11338 |
| Sequence 3362 | M69066 |
| Sequence 3363 | AF057299 |
| Sequence 3364 | D38555 |
| Sequence 3365 | AI050198 |
| Sequence 3366 | X17620 |
| Sequence 3367 | S72481 |
| Sequence 3368 | AF013759 |
| Sequence 3369 | L11005 |
| Sequence 3370 | D21262 |
| Sequence 3371 | AF054838 |
| Sequence 3372 | AB001872 |
| Sequence 3373 | M22918 |
| Sequence 3374 | AF064484 |
| Sequence 3375 | M16247 |
| Sequence 3376 | D50406 |
| Sequence 3377 | AF068754 |
| Sequence 3378 | J02853 |
| Sequence 3379 | AF061258 |
| Sequence 3380 | E03569 |
| Sequence 3381 | D28476 |
| Sequence 3382 | AF091263 |
| Sequence 3383 | AB032981 |
| Sequence 3384 | AF070600 |
| Sequence 3385 | M55409 |
| Sequence 3386 | AF152306 |
| Sequence 3387 | M60255 |
| Sequence 3388 | AF134159 |
| Sequence 3389 | AF188746 |
| Sequence 3390 | AF100742 |
| Sequence 3391 | AB032954 |
| Sequence 3392 | AF020736 |
| Sequence 3393 | J03537 |
| Sequence 3394 | S67815 |
| Sequence 3395 | AF077202 |
| Sequence 3396 | AL049969 |
| Sequence 3397 | AL049294 |
| Sequence 3398 | X53961 |
| Sequence 3399 | L34840 |
| Sequence 3400 | AF073887 |
| Sequence 3401 | X16940 |
| Sequence 3402 | AF086236 |
| Sequence 3403 | U37143 |
| Sequence 3404 | U70063 |
| Sequence 3405 | X56958 |
| Sequence 3406 | M23161 |
| Sequence 3407 | J04208 |
| Sequence 3408 | X74070 |
| Sequence 3409 | D50927 |
| Sequence 3410 | D13866 |
| Sequence 3411 | L38951 |
| Sequence 3412 | AF191018 |
| Sequence 3413 | D78611 |
| Sequence 3414 | AB021288 |
| Sequence 3415 | M97347 |
| Sequence 3416 | J05211 |
| Sequence 3417 | AF131844 |
| Sequence 3418 | AJ010953 |
| Sequence 3419 | U81006 |
| Sequence 3420 | E01979 |
| Sequence 3421 | AF067170 |
| Sequence 3422 | AF022229 |
| Sequence 3423 | AF108092 |
| Sequence 3424 | AF067972 |
| Sequence 3425 | AF098865 |
| Sequence 3426 | U07919 |
| Sequence 3427 | D14710 |
| Sequence 3428 | X74801 |
| Sequence 3429 | U02882 |
| Sequence 3430 | S77601 |
| Sequence 3431 | AL122076 |
| Sequence 3432 | D50926 |
| Sequence 3433 | AF151836 |
| Sequence 3434 | U51990 |
| Sequence 3435 | M17325 |
| Sequence 3436 | D50420 |
| Sequence 3437 | AF092134 |
| Sequence 3438 | X12451 |
| Sequence 3439 | M22414 |
| Sequence 3440 | AL050186 |
| Sequence 3441 | U25276 |
| Sequence 3442 | AF070652 |
| Sequence 3443 | S82081 |
| Sequence 3444 | L08599 |
| Sequence 3445 | AB028981 |
| Sequence 3446 | D49396 |
| Sequence 3447 | AF147339 |
| Sequence 3448 | U68140 |
| Sequence 3449 | AL110159 |
| Sequence 3450 | AF052180 |
| Sequence 3451 | AF020038 |
| Sequence 3452 | AF089747 |
| Sequence 3453 | AB018280 |
| Sequence 3454 | U92544 |
| Sequence 3455 | M35252 |
| Sequence 3456 | D86985 |
| Sequence 3457 | AF057706 |
| Sequence 3458 | AL079298 |
| Sequence 3459 | U51903 |
| Sequence 3460 | AF188747 |
| Sequence 3461 | AL117621 |
| Sequence 3462 | D10040 |
| Sequence 3463 | AF135488 |
| Sequence 3464 | Z34975 |
| Sequence 3465 | U15552 |
| Sequence 3466 | AF159056 |
| Sequence 3467 | U80735 |
| Sequence 3468 | D23662 |
| Sequence 3469 | X16896 |
| Sequence 3470 | M90360 |
| Sequence 3471 | U90919 |
| Sequence 3472 | AL050051 |
| Sequence 3473 | AF000364 |
| Sequence 3474 | AF005043 |
| Sequence 3475 | U15009 |
| Sequence 3476 | AF132000 |
| Sequence 3477 | AB007885 |
| Sequence 3478 | U49957 |
| Sequence 3479 | M65217 |
| Sequence 3480 | AB007883 |
| Sequence 3481 | U24105 |
| Sequence 3482 | M22382 |
| Sequence 3483 | D42044 |
| Sequence 3484 | U53468 |
| Sequence 3485 | M28372 |
| Sequence 3486 | AF058718 |
| Sequence 3487 | J03007 |
| Sequence 3488 | AL117430 |
| Sequence 3489 | AL080097 |
| Sequence 3490 | D63480 |
| Sequence 3491 | U61397 |
| Sequence 3492 | AF035319 |
| Sequence 3493 | AL110183 |
| Sequence 3494 | AF104914 |
| Sequence 3495 | AL050100 |
| Sequence 3496 | AB002382 |
| Sequence 3497 | U12778 |
| Sequence 3498 | D14665 |
| Sequence 3499 | Y13323 |
| Sequence 3500 | AF099989 |
| Sequence 3501 | D14662 |
| Sequence 3502 | AF023244 |
| Sequence 3503 | AL117666 |
| Sequence 3504 | AB016068 |
| Sequence 3505 | D00422 |
| Sequence 3506 | AB014547 |
| Sequence 3507 | J02923 |
| Sequence 3508 | AF078861 |
| Sequence 3509 | L37080 |
| Sequence 3510 | X55654 |
| Sequence 3511 | AF077034 |
| Sequence 3512 | U43604 |
| Sequence 3513 | M27689 |
| Sequence 3514 | AF047020 |
| Sequence 3515 | AF095770 |
| Sequence 3516 | AL109728 |
| Sequence 3517 | AL049470 |
| Sequence 3518 | M73837 |
| Sequence 3519 | X91257 |
| Sequence 3520 | AF081259 |
| Sequence 3521 | AF037447 |
| Sequence 3522 | M69181 |
| Sequence 3523 | M34840 |
| Sequence 3524 | D30037 |
| Sequence 3525 | X82125 |
| Sequence 3526 | AJ010046 |
| Sequence 3527 | AF102846 |
| Sequence 3528 | AL050187 |
| Sequence 3529 | AF132945 |
| Sequence 3530 | X92972 |
| Sequence 3531 | M22976 |
| Sequence 3532 | D87742 |
| Sequence 3533 | AF104012 |
| Sequence 3534 | D49489 |
| Sequence 3535 | U57847 |
| Sequence 3536 | AL035304 |
| Sequence 3537 | AF039918 |
| Sequence 3538 | AB033079 |
| Sequence 3539 | M74509 |
| Sequence 3540 | AL121735 |
| Sequence 3541 | AF171944 |
| Sequence 3542 | AB011004 |
| Sequence 3543 | AP155235 |
| Sequence 3544 | AB014888 |
| Sequence 3545 | M22920 |
| Sequence 3546 | D21092 |
| Sequence 3547 | AF004162 |
| Sequence 3548 | U00238 |
| Sequence 3549 | U50939 |
| Sequence 3550 | M24194 |
| Sequence 3551 | AF035293 |
| Sequence 3552 | AF151103 |
| Sequence 3553 | X80695 |
| Sequence 3554 | AL133076 |
| Sequence 3555 | AF069601 |
| Sequence 3556 | AF082657 |
| Sequence 3557 | AB019568 |
| Sequence 3558 | AL110181 |
| Sequence 3559 | D31886 |
| Sequence 3560 | AF084457 |
| Sequence 3561 | Y00281 |
| Sequence 3562 | U02493 |
| Sequence 3563 | X75535 |
| Sequence 3564 | AB002330 |
| Sequence 3565 | L19779 |
| Sequence 3566 | AP156965 |
| Sequence 3567 | AF151868 |
| Sequence 3568 | AF075061 |
| Sequence 3569 | AL050089 |
| Sequence 3570 | X76732 |
| Sequence 3571 | X05332 |
| Sequence 3572 | AF010312 |
| Sequence 3573 | AL050073 |
| Sequence 3574 | M76766 |
| Sequence 3575 | AJ002744 |
| Sequence 3576 | D13641 |
| Sequence 3577 | AF060219 |
| Sequence 3578 | AB014597 |
| Sequence 3579 | E01574 |
| Sequence 3580 | X74331 |
| Sequence 3581 | AL049932 |
| Sequence 3582 | D16562 |
| Sequence 3583 | AF078848 |
| Sequence 3584 | AJ223812 |
| Sequence 3585 | AL117392 |
| Sequence 3586 | L08437 |
| Sequence 3587 | AB023173 |
| Sequence 3588 | AP100755 |
| Sequence 3589 | D42045 |
| Sequence 3590 | D87442 |
| Sequence 3591 | AB033007 |
| Sequence 3592 | XI5187 |
| Sequence 3593 | D28540 |
| Sequence 3594 | Y15906 |
| Sequence 3595 | Y07593 |
| Sequence 3596 | U47674 |
| Sequence 3597 | X75861 |
| Sequence 3598 | D45198 |
| Sequence 3599 | U90551 |
| Sequence 3600 | AP151793 |
| Sequence 3601 | L12686 |
| Sequence 3602 | AF007216 |
| Sequence 3603 | AF086179 |
| Sequence 3604 | AB000888 |
| Sequence 3605 | AF013758 |
| Sequence 3606 | L19185 |
| Sequence 3607 | AP131784 |
| Sequence 3608 | M11353 |
| Sequence 3609 | AP100744 |
| Sequence 3610 | AJ010069 |
| Sequence 3611 | D14705 |
| Sequence 3612 | AF047442 |
| Sequence 3613 | Y09188 |
| Sequence 3614 | AF006086 |
| Sequence 3615 | D14043 |
| Sequence 3616 | AL110180 |
| Sequence 3617 | M16804 |
| Sequence 3618 | X60489 |
| Sequence 3619 | AB020658 |
| Sequence 3620 | X15729 |
| Sequence 3621 | U83857 |
| Sequence 3622 | AB018331 |
| Sequence 3623 | D10924 |
| Sequence 3624 | AB000095 |
| Sequence 3625 | D29011 |
| Sequence 3626 | J04183 |
| Sequence 3627 | AF068180 |
| Sequence 3628 | AF078855 |
| Sequence 3629 | J05594 |
| Sequence 3630 | AF078860 |
| Sequence 3631 | L38486 |
| Sequence 3632 | AF053970 |
| Sequence 3633 | AF086449 |
| Sequence 3634 | M18366 |
| Sequence 3635 | AF107405 |
| Sequence 3636 | D80001 |
| Sequence 3637 | M31627 |
| Sequence 3638 | AF000652 |
| Sequence 3639 | M69238 |
| Sequence 3640 | AF070649 |
| Sequence 3641 | E05957 |
| Sequence 3642 | AL050272 |
| Sequence 3643 | M64572 |
| Sequence 3644 | U36336 |
| Sequence 3645 | X94323 |
| Sequence 3646 | L13385 |
| Sequence 3647 | AB023196 |
| Sequence 3648 | AF000231 |
| Sequence 3649 | U21858 |
| Sequence 3650 | K01911 |
| Sequence 3651 | U58855 |
| Sequence 3652 | S74678 |
| Sequence 3653 | M10036 |
| Sequence 3654 | AF131814 |
| Sequence 3655 | D87444 |
| Sequence 3656 | AB028948 |
| Sequence 3657 | AF022212 |
| Sequence 3658 | D50405 |
| Sequence 3659 | AF033026 |
| Sequence 3660 | J03015 |
| Sequence 3661 | AB023179 |
| Sequence 3662 | AF022108 |
| Sequence 3663 | AF038404 |
| Sequence 3664 | AF086472 |
| Sequence 3665 | AB012910 |
| Sequence 3666 | D28473 |
| Sequence 3667 | X64838 |
| Sequence 3668 | D29954 |
| Sequence 3669 | J04615 |
| Sequence 3670 | AL049974 |
| Sequence 3671 | AL133108 |
| Sequence 3672 | AL050201 |
| Sequence 3673 | AB033091 |
| Sequence 3674 | D38522 |
| Sequence 3675 | U17714 |
| Sequence 3676 | AB018284 |
| Sequence 3677 | D14812 |
| Sequence 3678 | M34181 |
| Sequence 3679 | Z13009 |
| Sequence 3680 | L13773 |
| Sequence 3681 | AF027824 |
| Sequence 3682 | X82321 |
| Sequence 3683 | AB020694 |
| Sequence 3684 | S69272 |
| Sequence 3685 | AF070597 |
| Sequence 3686 | AF091090 |
| Sequence 3687 | U90548 |
| Sequence 3688 | V62427 |
| Sequence 3689 | V62430 |
| Sequence 3690 | V35457 |
| Sequence 3691 | V72211 |
| Sequence 3692 | X41346 |
| Sequence 3693 | Z15583 |
| Sequence 3694 | V58360 |
| Sequence 3695 | V90283 |
| Sequence 3696 | Q69214 |
| Sequence 3697 | V58756 |
| Sequence 3698 | X76578 |
| Sequence 3699 | V58510 |
| Sequence 3700 | V34176 |
| Sequence 3701 | Z08806 |
| Sequence 3702 | V58544 |
| Sequence 3703 | V29267 |
| Sequence 3704 | T59274 |
| Sequence 3705 | X27262 |
| Sequence 3706 | X40518 |
| Sequence 3707 | V62428 |
| Sequence 3708 | V99733 |
| Sequence 3709 | T72167 |
| Sequence 3710 | V58628 |
| Sequence 3711 | X03841 |
| Sequence 3712 | V70354 |
| Sequence 3713 | V58523 |
| Sequence 3714 | Z27248 |
| Sequence 3715 | X26850 |
| Sequence 3716 | X26844 |
| Sequence 3717 | V36503 |
| Sequence 3718 | V59613 |
| Sequence 3719 | Z33428 |
| Sequence 3720 | V57903 |
| Sequence 3721 | Z15132 |
| Sequence 3722 | V84510 |
| Sequence 3723 | AC02230 |
| Sequence 3724 | AC33541 |
| Sequence 3725 | AC34175 |
| Sequence 3726 | AC34877 |
| Sequence 3727 | AC36715 |
| Sequence 3728 | AC36117 |
| Sequence 3729 | AC36739 |
| Sequence 3730 | AC36708 |
| Sequence 3731 | AC36079 |
| Sequence 3732 | AC36078 |
| Sequence 3733 | AC36740 |
| Sequence 3734 | A21185 |
| Sequence 3735 | AB000220 |
| Sequence 3736 | AB002303 |
| Sequence 3737 | AB002369 |
| Sequence 3738 | AB002387 |
| Sequence 3739 | AB007510 |
| Sequence 3740 | AB007884 |
| Sequence 3741 | AB007956 |
| Sequence 3742 | AB011004 |
| Sequence 3743 | AB014531 |
| Sequence 3744 | AB014597 |
| Sequence 3745 | AB016068 |
| Sequence 3746 | AB018080 |
| Sequence 3747 | AB018284 |
| Sequence 3748 | AB023230 |
| Sequence 3749 | AB032957 |
| Sequence 3750 | AB032983 |
| Sequence 3751 | AB033007 |
| Sequence 3752 | AB033078 |
| Sequence 3753 | AB033079 |
| Sequence 3754 | AB033091 |
| Sequence 3755 | AF000231 |
| Sequence 3756 | AF001212 |
| Sequence 3757 | AF007144 |
| Sequence 3758 | AF007216 |
| Sequence 3759 | AF007217 |
| Sequence 3760 | AF010233 |
| Sequence 3761 | AF010312 |
| Sequence 3762 | AF012126 |
| Sequence 3763 | AF012549 |
| Sequence 3764 | AF014955 |
| Sequence 3765 | AF020202 |
| Sequence 3766 | AF020736 |
| Sequence 3767 | AF020761 |
| Sequence 3768 | AF026166 |
| Sequence 3769 | AF028832 |
| Sequence 3770 | AF030403 |
| Sequence 3771 | AF038404 |
| Sequence 3772 | AF039918 |
| Sequence 3773 | AF044956 |
| Sequence 3774 | AF047020 |
| Sequence 3775 | AF054838 |
| Sequence 3776 | AF060219 |
| Sequence 3777 | AF061258 |
| Sequence 3778 | AF065388 |
| Sequence 3779 | AF070553 |
| Sequence 3780 | AF071202 |
| Sequence 3781 | AF077037 |
| Sequence 3782 | AF077042 |
| Sequence 3783 | AF078849 |
| Sequence 3784 | AF081281 |
| Sequence 3785 | AF084555 |
| Sequence 3786 | AF085845 |
| Sequence 3787 | AF086477 |
| Sequence 3788 | AF092133 |
| Sequence 3789 | AF092565 |
| Sequence 3790 | AF103907 |
| Sequence 3791 | AF132937 |
| Sequence 3792 | AF134159 |
| Sequence 3793 | AF144566 |
| Sequence 3794 | AF146277 |
| Sequence 3795 | AF147339 |
| Sequence 3796 | AF151103 |
| Sequence 3797 | AF152306 |
| Sequence 3798 | AF156965 |
| Sequence 3799 | AF159056 |
| Sequence 3800 | AF170583 |
| Sequence 3801 | AF176574 |
| Sequence 3802 | AF188745 |
| Sequence 3803 | AJ001443 |
| Sequence 3804 | AJ002744 |
| Sequence 3805 | AJ004913 |
| Sequence 3806 | AJ010069 |
| Sequence 3807 | AJ223812 |
| Sequence 3808 | AJ224875 |
| Sequence 3809 | AL035304 |
| Sequence 3810 | AL049229 |
| Sequence 3811 | AL049274 |
| Sequence 3812 | AL049932 |
| Sequence 3813 | AL049941 |
| Sequence 3814 | AL049969 |
| Sequence 3815 | AL050073 |
| Sequence 3816 | AL080084 |
| Sequence 3817 | AL110126 |
| Sequence 3818 | AL117392 |
| Sequence 3819 | AL117423 |
| Sequence 3820 | AL117576 |
| Sequence 3821 | AL117644 |
| Sequence 3822 | AL117665 |
| Sequence 3823 | D00039 |
| Sequence 3824 | D13641 |
| Sequence 3825 | D13866 |
| Sequence 3826 | D14043 |
| Sequence 3827 | D16562 |
| Sequence 3828 | D29954 |
| Sequence 3829 | D38305 |
| Sequence 3830 | D80005 |
| Sequence 3831 | D87742 |
| Sequence 3832 | D88208 |
| Sequence 3833 | D90226 |
| Sequence 3834 | E01956 |
| Sequence 3835 | E02628 |
| Sequence 3836 | E03569 |
| Sequence 3837 | E07798 |
| Sequence 3838 | J02923 |
| Sequence 3839 | J04205 |
| Sequence 3840 | J04794 |
| Sequence 3841 | J05192 |
| Sequence 3842 | K01911 |
| Sequence 3843 | L07033 |
| Sequence 3844 | L38961 |
| Sequence 3845 | M10036 |
| Sequence 3846 | M12938 |
| Sequence 3847 | M17885 |
| Sequence 3848 | M20260 |
| Sequence 3849 | M21895 |
| Sequence 3850 | M24194 |
| Sequence 3851 | M24902 |
| Sequence 3852 | M26663 |
| Sequence 3853 | M29366 |
| Sequence 3854 | M30938 |
| Sequence 3855 | M34840 |
| Sequence 3856 | M36634 |
| Sequence 3857 | M97934 |
| Sequence 3858 | S82081 |
| Sequence 3859 | U01923 |
| Sequence 3860 | U02680 |
| Sequence 3861 | U17104 |
| Sequence 3862 | U33821 |
| Sequence 3863 | U34252 |
| Sequence 3864 | U36336 |
| Sequence 3865 | U50203 |
| Sequence 3866 | U51990 |
| Sequence 3867 | X04408 |
| Sequence 3868 | X05332 |
| Sequence 3869 | X15729 |
| Sequence 3870 | X55654 |
| Sequence 3871 | X67698 |
| Sequence 3872 | X74331 |
| Sequence 3873 | X75535 |
| Sequence 3874 | X75861 |
| Sequence 3875 | X80910 |
| Sequence 3876 | X87241 |
| Sequence 3877 | X94323 |
| Sequence 3878 | X94910 |
| Sequence 3879 | X98296 |
| Sequence 3880 | Y00345 |
| Sequence 3881 | Z11531 |
| Sequence 3882 | Z26317 |
| Sequence 3883 | Z46606 |
| Sequence 3884 | Z48605 |
| Sequence 3885 | AA001734 |
| Sequence 3886 | AA002140 |
| Sequence 3887 | AA026797 |
| Sequence 3888 | AA041467 |
| Sequence 3889 | AA043109 |
| Sequence 3890 | AA045803 |
| Sequence 3891 | AA047482 |
| Sequence 3892 | AA056437 |
| Sequence 3893 | AA058899 |
| Sequence 3894 | AA081003 |
| Sequence 3895 | AA083026 |
| Sequence 3896 | AA085835 |
| Sequence 3897 | AA091277 |
| Sequence 3898 | AA094000 |
| Sequence 3899 | AA095049 |
| Sequence 3900 | AA111855 |
| Sequence 3901 | AA113811 |
| Sequence 3902 | AA126472 |
| Sequence 3903 | AA135001 |
| Sequence 3904 | AA136221 |
| Sequence 3905 | AA136404 |
| Sequence 3906 | AA148087 |
| Sequence 3907 | AA148831 |
| Sequence 3908 | AA149579 |
| Sequence 3909 | AA149979 |
| Sequence 3910 | AA160149 |
| Sequence 3911 | AA167696 |
| Sequence 3912 | AA167829 |
| Sequence 3913 | AA171605 |
| Sequence 3914 | AA180845 |
| Sequence 3915 | AA187604 |
| Sequence 3916 | AA191536 |
| Sequence 3917 | AA193086 |
| Sequence 3918 | AA195010 |
| Sequence 3919 | AA196978 |
| Sequence 3920 | AA203537 |
| Sequence 3921 | AA215793 |
| Sequence 3922 | AA216690 |
| Sequence 3923 | AA225294 |
| Sequence 3924 | AA226171 |
| Sequence 3925 | AA227000 |
| Sequence 3926 | AA228273 |
| Sequence 3927 | AA228940 |
| Sequence 3928 | AA229495 |
| Sequence 3929 | AA243809 |
| Sequence 3930 | AA250947 |
| Sequence 3931 | AA256117 |
| Sequence 3932 | AA257955 |
| Sequence 3933 | AA261819 |
| Sequence 3934 | AA280647 |
| Sequence 3935 | AA282286 |
| Sequence 3936 | AA285341 |
| Sequence 3937 | AA297647 |
| Sequence 3938 | AA306876 |
| Sequence 3939 | AA307103 |
| Sequence 3940 | AA308578 |
| Sequence 3941 | AA310637 |
| Sequence 3942 | AA311819 |
| Sequence 3943 | AA312495 |
| Sequence 3944 | AA312968 |
| Sequence 3945 | AA314907 |
| Sequence 3946 | AA315555 |
| Sequence 3947 | AA316159 |
| Sequence 3948 | AA326359 |
| Sequence 3949 | AA326833 |
| Sequence 3950 | AA334710 |
| Sequence 3951 | AA336412 |
| Sequence 3952 | AA345904 |
| Sequence 3953 | AA346844 |
| Sequence 3954 | AA355553 |
| Sequence 3955 | AA359832 |
| Sequence 3956 | AA368792 |
| Sequence 3957 | AA375531 |
| Sequence 3958 | AA399218 |
| Sequence 3959 | AA399606 |
| Sequence 3960 | AA418628 |
| Sequence 3961 | AA418770 |
| Sequence 3962 | AA420680 |
| Sequence 3963 | AA424456 |
| Sequence 3964 | AA426351 |
| Sequence 3965 | AA430304 |
| Sequence 3966 | AA436125 |
| Sequence 3967 | AA437099 |
| Sequence 3968 | AA437224 |
| Sequence 3969 | AA442479 |
| Sequence 3970 | AA447322 |
| Sequence 3971 | AA455917 |
| Sequence 3972 | AA460587 |
| Sequence 3973 | AA460649 |
| Sequence 3974 | AA461015 |
| Sequence 3975 | AA464133 |
| Sequence 3976 | AA469238 |
| Sequence 3977 | AA471025 |
| Sequence 3978 | AA480124 |
| Sequence 3979 | AA488732 |
| Sequence 3980 | AA489173 |
| Sequence 3981 | AA492192 |
| Sequence 3982 | AA497050 |
| Sequence 3983 | AA501968 |
| Sequence 3984 | AA502799 |
| Sequence 3985 | AA502979 |
| Sequence 3986 | AA516531 |
| Sequence 3987 | AA523498 |
| Sequence 3988 | AA523902 |
| Sequence 3989 | AA524651 |
| Sequence 3990 | AA528104 |
| Sequence 3991 | AA531249 |
| Sequence 3992 | AA533553 |
| Sequence 3993 | AA541703 |
| Sequence 3994 | AA543102 |
| Sequence 3995 | AA548472 |
| Sequence 3996 | AA548722 |
| Sequence 3997 | AA569813 |
| Sequence 3998 | AA570171 |
| Sequence 3999 | AA576425 |
| Sequence 4000 | AA578773 |
| Sequence 4001 | AA580771 |
| Sequence 4002 | AA608967 |
| Sequence 4003 | AA635590 |
| Sequence 4004 | AA639786 |
| Sequence 4005 | AA640474 |
| Sequence 4006 | AA652478 |
| Sequence 4007 | AA662733 |
| Sequence 4008 | AA680052 |
| Sequence 4009 | AA701667 |
| Sequence 4010 | AA706224 |
| Sequence 4011 | AA748031 |
| Sequence 4012 | AA770006 |
| Sequence 4013 | AA779670 |
| Sequence 4014 | AA810905 |
| Sequence 4015 | AA813941 |
| Sequence 4016 | AA860839 |
| Sequence 4017 | AA890010 |
| Sequence 4018 | AA890326 |
| Sequence 4019 | AA902625 |
| Sequence 4020 | AA971532 |
| Sequence 4021 | AA975275 |
| Sequence 4022 | AA984384 |
| Sequence 4023 | AA988520 |
| Sequence 4024 | AA992583 |
| Sequence 4025 | AF114027 |
| Sequence 4026 | AF150208 |
| Sequence 4027 | AI041116 |
| Sequence 4028 | AI051210 |
| Sequence 4029 | AI064967 |
| Sequence 4030 | AI075338 |
| Sequence 4031 | AI076042 |
| Sequence 4032 | AI080267 |
| Sequence 4033 | AI129991 |
| Sequence 4034 | AI133582 |
| Sequence 4035 | AI147472 |
| Sequence 4036 | AI149363 |
| Sequence 4037 | AI188850 |
| Sequence 4038 | A1198311 |
| Sequence 4039 | AI198937 |
| Sequence 4040 | AI199681 |
| Sequence 4041 | AI201248 |
| Sequence 4042 | AI221390 |
| Sequence 4043 | AI263876 |
| Sequence 4044 | AI267162 |
| Sequence 4045 | AI267185 |
| Sequence 4046 | AI267216 |
| Sequence 4047 | AI267254 |
| Sequence 4048 | AI267282 |
| Sequence 4049 | AI278643 |
| Sequence 4050 | AI300562 |
| Sequence 4051 | AI336326 |
| Sequence 4052 | AI361256 |
| Sequence 4053 | AI367759 |
| Sequence 4054 | AI431801 |
| Sequence 4055 | AI472067 |
| Sequence 4056 | AI547327 |
| Sequence 4057 | AI557182 |
| Sequence 4058 | AI557225 |
| Sequence 4059 | AI557235 |
| Sequence 4060 | AI557277 |
| Sequence 4061 | AI564487 |
| Sequence 4062 | AI620427 |
| Sequence 4063 | AI627356 |
| Sequence 4064 | AI630306 |
| Sequence 4065 | AI630357 |
| Sequence 4066 | AI630362 |
| Sequence 4067 | AI633646 |
| Sequence 4068 | AI656122 |
| Sequence 4069 | AI658850 |
| Sequence 4070 | AI682937 |
| Sequence 4071 | AI686139 |
| Sequence 4072 | AI694767 |
| Sequence 4073 | AI741213 |
| Sequence 4074 | AI743852 |
| Sequence 4075 | AI755085 |
| Sequence 4076 | AI766083 |
| Sequence 4077 | AI767692 |
| Sequence 4078 | AI799891 |
| Sequence 4079 | AI825408 |
| Sequence 4080 | AI826504 |
| Sequence 4081 | AI912057 |
| Sequence 4082 | AI914133 |
| Sequence 4083 | AI925735 |
| Sequence 4084 | AI949242 |
| Sequence 4085 | AI953152 |
| Sequence 4086 | AI968401 |
| Sequence 4087 | AI970381 |
| Sequence 4088 | AL036483 |
| Sequence 4089 | AL042404 |
| Sequence 4090 | AL043046 |
| Sequence 4091 | AL047415 |
| Sequence 4092 | AL047881 |
| Sequence 4093 | AW022756 |
| Sequence 4094 | AW044142 |
| Sequence 4095 | AW068579 |
| Sequence 4096 | AW149817 |
| Sequence 4097 | AW160731 |
| Sequence 4098 | AW163485 |
| Sequence 4099 | AW176022 |
| Sequence 4100 | AW179322 |
| Sequence 4101 | C06419 |
| Sequence 4102 | H00112 |
| Sequence 4103 | H03785 |
| Sequence 4104 | H06944 |
| Sequence 4105 | H13891 |
| Sequence 4106 | H15654 |
| Sequence 4107 | H17741 |
| Sequence 4108 | H54240 |
| Sequence 4109 | H78517 |
| Sequence 4110 | H78888 |
| Sequence 4111 | H79639 |
| Sequence 4112 | N23545 |
| Sequence 4113 | N27218 |
| Sequence 4114 | N58818 |
| Sequence 4115 | N62448 |
| Sequence 4116 | R35529 |
| Sequence 4117 | R55535 |
| Sequence 4118 | R72738 |
| Sequence 4119 | T08676 |
| Sequence 4120 | T79397 |
| Sequence 4121 | T79514 |
| Sequence 4122 | W15195 |
| Sequence 4123 | W19757 |
| Sequence 4124 | W25081 |
| Sequence 4125 | W56069 |
| Sequence 4126 | W67735 |
| Sequence 4127 | W79779 |
| Sequence 4128 | W88856 |
| Sequence 4129 | T99948 |
| Sequence 4130 | V54589 |
| Sequence 4131 | V58628 |
| Sequence 4132 | V63195 |
| Sequence 4133 | X03841 |
| Sequence 4134 | X04318 |
| Sequence 4135 | X26850 |
| Sequence 4136 | X27262 |
| Sequence 4137 | X27278 |
| Sequence 4138 | X39870 |
| Sequence 4139 | X97676 |
| Sequence 4140 | Z15277 |
| Sequence 4141 | Z27248 |
| Sequence 4142 | Z33526 |
| Sequence 4143 | Z41241 |
| Sequence 4144 | AC34465 |
| Sequence 4145 | AC36078 |
| Sequence 4146 | AC36079 |
| Sequence 4147 | AC36099 |
| Sequence 4148 | AC36175 |
| Sequence 4149 | AC36180 |
| Sequence 4150 | AC36201 |
| Sequence 4151 | AC36457 |
| Sequence 4152 | AC36740 |
| Sequence 4153 | A6020866 |
| Sequence 4154 | AC002040 |
| Sequence 4155 | AC002064 |
| Sequence 4156 | AC003991 |
| Sequence 4157 | AC004656 |
| Sequence 4158 | AC005154 |
| Sequence 4159 | AC005538 |
| Sequence 4160 | AC005831 |
| Sequence 4161 | AC007052 |
| Sequence 4162 | AC007686 |
| Sequence 4163 | AF061258 |
| Sequence 4164 | AL034399 |
| Sequence 4165 | AL035666 |
| Sequence 4166 | AL049821 |
| Sequence 4167 | AP000527 |
| Sequence 4168 | AP000528 |
| Sequence 4169 | AP000529 |
| Sequence 4170 | G16768 |
| Sequence 4171 | S65371 |
| Sequence 4172 | U66083 |
| Sequence 4173 | X70394 |
| Sequence 4174 | Z94388 |
| Sequence 4175 | AW235635 |
| Sequence 4176 | AI383637 |
| Sequence 4177 | AC005480 |
| Sequence 4178 | AL031003 |
| Sequence 4179 | AB020866 |
| Sequence 4180 | S65371 |
| Sequence 4181 | AA001673 |
| Sequence 4182 | AA002131 |
| Sequence 4183 | AA007370 |
| Sequence 4184 | AA009611 |
| Sequence 4185 | AA010689 |
| Sequence 4186 | AA010981 |
| Sequence 4187 | AA021608 |
| Sequence 4188 | AA024581 |
| Sequence 4189 | AA026399 |
| Sequence 4190 | AA029143 |
| Sequence 4191 | AA029567 |
| Sequence 4192 | AA029889 |
| Sequence 4193 | AA031360 |
| Sequence 4194 | AA032221 |
| Sequence 4195 | AA035001 |
| Sequence 4196 | AA035773 |
| Sequence 4197 | AA037425 |
| Sequence 4198 | AA037620 |
| Sequence 4199 | AA039807 |
| Sequence 4200 | AA039998 |
| Sequence 4201 | AA040180 |
| Sequence 4202 | AA040710 |
| Sequence 4203 | AA044118 |
| Sequence 4204 | AA044586 |
| Sequence 4205 | AA046083 |
| Sequence 4206 | AA047432 |
| Sequence 4207 | AA053265 |
| Sequence 4208 | AA053932 |
| Sequence 4209 | AA056345 |
| Sequence 4210 | AA057063 |
| Sequence 4211 | AA057270 |
| Sequence 4212 | AA057490 |
| Sequence 4213 | AA057861 |
| Sequence 4214 | AA062731 |
| Sequence 4215 | AA062859 |
| Sequence 4216 | AA070349 |
| Sequence 4217 | AA074142 |
| Sequence 4218 | AA074729 |
| Sequence 4219 | AA074869 |
| Sequence 4220 | AA075133 |
| Sequence 4221 | AA075459 |
| Sequence 4222 | AA081007 |
| Sequence 4223 | AA081355 |
| Sequence 4224 | AA082685 |
| Sequence 4225 | AA083479 |
| Sequence 4226 | AA084987 |
| Sequence 4227 | AA085483 |
| Sequence 4228 | AA088372 |
| Sequence 4229 | AA088770 |
| Sequence 4230 | AA090106 |
| Sequence 4231 | AA093276 |
| Sequence 4232 | AA094656 |
| Sequence 4233 | AA096121 |
| Sequence 4234 | AA099976 |
| Sequence 4235 | AA100852 |
| Sequence 4236 | AA100876 |
| Sequence 4237 | AA112310 |
| Sequence 4238 | AA114062 |
| Sequence 4239 | AA114854 |
| Sequence 4240 | AA114967 |
| Sequence 4241 | AA115021 |
| Sequence 4242 | AA115492 |
| Sequence 4243 | AA120816 |
| Sequence 4244 | AA121127 |
| Sequence 4245 | AA121419 |
| Sequence 4246 | AA122237 |
| Sequence 4247 | AA122245 |
| Sequence 4248 | AA125817 |
| Sequence 4249 | AA126121 |
| Sequence 4250 | AA126729 |
| Sequence 4251 | AA126809 |
| Sequence 4252 | AA126921 |
| Sequence 4253 | AA127029 |
| Sequence 4254 | AA127714 |
| Sequence 4255 | AA128387 |
| Sequence 4256 | AA128442 |
| Sequence 4257 | AA129615 |
| Sequence 4258 | AA129652 |
| Sequence 4259 | AA129781 |
| Sequence 4260 | AA130162 |
| Sequence 4261 | AA130515 |
| Sequence 4262 | AA130529 |
| Sequence 4263 | AA130928 |
| Sequence 4264 | AA131313 |
| Sequence 4265 | AA131812 |
| Sequence 4266 | AA131926 |
| Sequence 4267 | AA132689 |
| Sequence 4268 | AA132844 |
| Sequence 4269 | AA133030 |
| Sequence 4270 | AA133361 |
| Sequence 4271 | AA133652 |
| Sequence 4272 | AA134022 |
| Sequence 4273 | AA134203 |
| Sequence 4274 | AA135870 |
| Sequence 4275 | AA136215 |
| Sequence 4276 | AA137165 |
| Sequence 4277 | AA143252 |
| Sequence 4278 | AA143578 |
| Sequence 4279 | AA146582 |
| Sequence 4280 | AA147608 |
| Sequence 4281 | AA147980 |
| Sequence 4282 | AA148729 |
| Sequence 4283 | AA149337 |
| Sequence 4284 | AA149492 |
| Sequence 4285 | AA149979 |
| Sequence 4286 | AA150891 |
| Sequence 4287 | AA151018 |
| Sequence 4288 | AA151506 |
| Sequence 4289 | AA156092 |
| Sequence 4290 | AA156879 |
| Sequence 4291 | AA158507 |
| Sequence 4292 | AA159181 |
| Sequence 4293 | AA159272 |
| Sequence 4294 | AA165164 |
| Sequence 4295 | AA167011 |
| Sequence 4296 | AA167150 |
| Sequence 4297 | AA167674 |
| Sequence 4298 | AA169374 |
| Sequence 4299 | AA169794 |
| Sequence 4300 | AA171510 |
| Sequence 4301 | AA171872 |
| Sequence 4302 | AA171883 |
| Sequence 4303 | AA173363 |
| Sequence 4304 | AA173997 |
| Sequence 4305 | AA176331 |
| Sequence 4306 | AA177127 |
| Sequence 4307 | AA180137 |
| Sequence 4308 | AA186346 |
| Sequence 4309 | AA186399 |
| Sequence 4310 | AA186733 |
| Sequence 4311 | AA187383 |
| Sequence 4312 | AA188045 |
| Sequence 4313 | AA188052 |
| Sequence 4314 | AA188169 |
| Sequence 4315 | AA190561 |
| Sequence 4316 | AA191134 |
| Sequence 4317 | AA191552 |
| Sequence 4318 | AA193346 |
| Sequence 4319 | AA194517 |
| Sequence 4320 | AA194860 |
| Sequence 4321 | AA195010 |
| Sequence 4322 | AA195044 |
| Sequence 4323 | AA195063 |
| Sequence 4324 | AA195145 |
| Sequence 4325 | AA196558 |
| Sequence 4326 | AA199802 |
| Sequence 4327 | AA203592 |
| Sequence 4328 | AA204701 |
| Sequence 4329 | AA204781 |
| Sequence 4330 | AA205647 |
| Sequence 4331 | AA205870 |
| Sequence 4332 | AA213619 |
| Sequence 4333 | AA214710 |
| Sequence 4334 | AA215628 |
| Sequence 4335 | AA218936 |
| Sequence 4336 | AA219705 |
| Sequence 4337 | AA223306 |
| Sequence 4338 | AA224124 |
| Sequence 4339 | AA225865 |
| Sequence 4340 | AA226137 |
| Sequence 4341 | AA226171 |
| Sequence 4342 | AA227506 |
| Sequence 4343 | AA227846 |
| Sequence 4344 | AA228273 |
| Sequence 4345 | AA229183 |
| Sequence 4346 | AA229495 |
| Sequence 4347 | AA229513 |
| Sequence 4348 | AA229993 |
| Sequence 4349 | AA232691 |
| Sequence 4350 | AA233689 |
| Sequence 4351 | AA233868 |
| Sequence 4352 | AA234130 |
| Sequence 4353 | AA236057 |
| Sequence 4354 | AA236323 |
| Sequence 4355 | AA243361 |
| Sequence 4356 | AA247816 |
| Sequence 4357 | AA251303 |
| Sequence 4358 | AA251644 |
| Sequence 4359 | AA252355 |
| Sequence 4360 | AA255695 |
| Sequence 4361 | AA255717 |
| Sequence 4362 | AA256330 |
| Sequence 4363 | AA256757 |
| Sequence 4364 | AA262296 |
| Sequence 4365 | AA279347 |
| Sequence 4366 | AA279949 |
| Sequence 4367 | AA280694 |
| Sequence 4368 | AA280735 |
| Sequence 4369 | AA283704 |
| Sequence 4370 | AA284161 |
| Sequence 4371 | AA284494 |
| Sequence 4372 | AA284555 |
| Sequence 4373 | AA287751 |
| Sequence 4374 | AA287961 |
| Sequence 4375 | AA291233 |
| Sequence 4376 | AA291756 |
| Sequence 4377 | AA292692 |
| Sequence 4378 | AA292993 |
| Sequence 4379 | AA296846 |
| Sequence 4380 | AA297434 |
| Sequence 4381 | AA297883 |
| Sequence 4382 | AA298489 |
| Sequence 4383 | AA298786 |
| Sequence 4384 | AA298807 |
| Sequence 4385 | AA299830 |
| Sequence 4386 | AA300065 |
| Sequence 4387 | AA302852 |
| Sequence 4388 | AA302919 |
| Sequence 4389 | AA306206 |
| Sequence 4390 | AA306888 |
| Sequence 4391 | AA307209 |
| Sequence 4392 | AA307247 |
| Sequence 4393 | AA307789 |
| Sequence 4394 | AA309445 |
| Sequence 4395 | AA310663 |
| Sequence 4396 | AA310739 |
| Sequence 4397 | AA312756 |
| Sequence 4398 | AA313223 |
| Sequence 4399 | AA314022 |
| Sequence 4400 | AA314127 |
| Sequence 4401 | AA314188 |
| Sequence 4402 | AA314673 |
| Sequence 4403 | AA314872 |
| Sequence 4404 | AA315095 |
| Sequence 4405 | AA315259 |
| Sequence 4406 | AA315805 |
| Sequence 4407 | AA315838 |
| Sequence 4408 | AA316627 |
| Sequence 4409 | AA318745 |
| Sequence 4410 | AA320004 |
| Sequence 4411 | AA322115 |
| Sequence 4412 | AA325145 |
| Sequence 4413 | AA325826 |
| Sequence 4414 | AA330090 |
| Sequence 4415 | AA333526 |
| Sequence 4416 | AA334457 |
| Sequence 4417 | AA334754 |
| Sequence 4418 | AA335699 |
| Sequence 4419 | AA336586 |
| Sequence 4420 | AA344743 |
| Sequence 4421 | AA345624 |
| Sequence 4422 | AA349118 |
| Sequence 4423 | AA352131 |
| Sequence 4424 | AA353766 |
| Sequence 4425 | AA356682 |
| Sequence 4426 | AA357811 |
| Sequence 4427 | AA358450 |
| Sequence 4428 | AA361403 |
| Sequence 4429 | AA366865 |
| Sequence 4430 | AA367471 |
| Sequence 4431 | AA368520 |
| Sequence 4432 | AA370337 |
| Sequence 4433 | AA370352 |
| Sequence 4434 | AA371017 |
| Sequence 4435 | AA371265 |
| Sequence 4436 | AA371626 |
| Sequence 4437 | AA375853 |
| Sequence 4438 | AA375988 |
| Sequence 4439 | AA376870 |
| Sequence 4440 | AA393480 |
| Sequence 4441 | AA394298 |
| Sequence 4442 | AA398686 |
| Sequence 4443 | AA399133 |
| Sequence 4444 | AA401339 |
| Sequence 4445 | AA401480 |
| Sequence 4446 | AA402095 |
| Sequence 4447 | AA403051 |
| Sequence 4448 | AA405663 |
| Sequence 4449 | AA406475 |
| Sequence 4450 | AA410580 |
| Sequence 4451 | AA418796 |
| Sequence 4452 | AA420845 |
| Sequence 4453 | AA421021 |
| Sequence 4454 | AA421562 |
| Sequence 4455 | AA425394 |
| Sequence 4456 | AA426351 |
| Sequence 4457 | AA427477 |
| Sequence 4458 | AA429912 |
| Sequence 4459 | AA431677 |
| Sequence 4460 | AA436377 |
| Sequence 4461 | AA437224 |
| Sequence 4462 | AA441885 |
| Sequence 4463 | AA441918 |
| Sequence 4464 | AA441959 |
| Sequence 4465 | AA442287 |
| Sequence 4466 | AA442372 |
| Sequence 4467 | AA442829 |
| Sequence 4468 | AA443750 |
| Sequence 4469 | AA443790 |
| Sequence 4470 | AA443946 |
| Sequence 4471 | AA444074 |
| Sequence 4472 | AA445955 |
| Sequence 4473 | AA446116 |
| Sequence 4474 | AA446149 |
| Sequence 4475 | AA447322 |
| Sequence 4476 | AA447948 |
| Sequence 4477 | AA448036 |
| Sequence 4478 | AA448531 |
| Sequence 4479 | AA449604 |
| Sequence 4480 | AA451633 |
| Sequence 4481 | AA453612 |
| Sequence 4482 | AA454208 |
| Sequence 4483 | AA458514 |
| Sequence 4484 | AA459617 |
| Sequence 4485 | AA460120 |
| Sequence 4486 | AA461082 |
| Sequence 4487 | AA461548 |
| Sequence 4488 | AA463198 |
| Sequence 4489 | AA464133 |
| Sequence 4490 | AA465193 |
| Sequence 4491 | AA465258 |
| Sequence 4492 | AA465293 |
| Sequence 4493 | AA465387 |
| Sequence 4494 | AA468839 |
| Sequence 4495 | AA469151 |
| Sequence 4496 | AA478514 |
| Sequence 4497 | AA479327 |
| Sequence 4498 | AA481001 |
| Sequence 4499 | AA481303 |
| Sequence 4500 | AA482654 |
| Sequence 4501 | AA485861 |
| Sequence 4502 | AA485906 |
| Sequence 4503 | AA485996 |
| Sequence 4504 | AA486628 |
| Sequence 4505 | AA487261 |
| Sequence 4506 | AA488227 |
| Sequence 4507 | AA488744 |
| Sequence 4508 | AA488809 |
| Sequence 4509 | AA489020 |
| Sequence 4510 | AA489433 |
| Sequence 4511 | AA492042 |
| Sequence 4512 | AA492280 |
| Sequence 4513 | AA493512 |
| Sequence 4514 | AA495759 |
| Sequence 4515 | AA495938 |
| Sequence 4516 | AA502979 |
| Sequence 4517 | AA503182 |
| Sequence 4518 | AA503215 |
| Sequence 4519 | AA503265 |
| Sequence 4520 | AA503731 |
| Sequence 4521 | AA504238 |
| Sequence 4522 | AA504340 |
| Sequence 4523 | AA505198 |
| Sequence 4524 | AA508588 |
| Sequence 4525 | AA508889 |
| Sequence 4526 | AA508902 |
| Sequence 4527 | AA512974 |
| Sequence 4528 | AA513177 |
| Sequence 4529 | AA513688 |
| Sequence 4530 | AA515857 |
| Sequence 4531 | AA516531 |
| Sequence 4532 | AA522481 |
| Sequence 4533 | AA522856 |
| Sequence 4534 | AA522867 |
| Sequence 4535 | AA523252 |
| Sequence 4536 | AA523464 |
| Sequence 4537 | AA523484 |
| Sequence 4538 | AA523938 |
| Sequence 4539 | AA524812 |
| Sequence 4540 | AA526886 |
| Sequence 4541 | AA527730 |
| Sequence 4542 | AA527805 |
| Sequence 4543 | AA528104 |
| Sequence 4544 | AA531563 |
| Sequence 4545 | AA533553 |
| Sequence 4546 | AA533772 |
| Sequence 4547 | AA534171 |
| Sequence 4548 | AA534418 |
| Sequence 4549 | AA534540 |
| Sequence 4550 | AA534933 |
| Sequence 4551 | AA535837 |
| Sequence 4552 | AA541677 |
| Sequence 4553 | AA541678 |
| Sequence 4554 | AA541808 |
| Sequence 4555 | AA542906 |
| Sequence 4556 | AA548238 |
| Sequence 4557 | AA551146 |
| Sequence 4558 | AA565420 |
| Sequence 4559 | AA565882 |
| Sequence 4560 | AA569130 |
| Sequence 4561 | AA569809 |
| Sequence 4562 | AA573824 |
| Sequence 4563 | AA574288 |
| Sequence 4564 | AA574397 |
| Sequence 4565 | AA576425 |
| Sequence 4566 | AA578773 |
| Sequence 4567 | AA580900 |
| Sequence 4568 | AA581117 |
| Sequence 4569 | AA582252 |
| Sequence 4570 | AA582596 |
| Sequence 4571 | AA588474 |
| Sequence 4572 | AA600998 |
| Sequence 4573 | AA602430 |
| Sequence 4574 | AA602794 |
| Sequence 4575 | AA603334 |
| Sequence 4576 | AA604474 |
| Sequence 4577 | AA612666 |
| Sequence 4578 | AA612829 |
| Sequence 4579 | AA612874 |
| Sequence 4580 | AA618277 |
| Sequence 4581 | AA620750 |
| Sequence 4582 | AA625951 |
| Sequence 4583 | AA626509 |
| Sequence 4584 | AA628417 |
| Sequence 4585 | AA629027 |
| Sequence 4586 | AA631868 |
| Sequence 4587 | AA639059 |
| Sequence 4588 | AA639174 |
| Sequence 4589 | AA639791 |
| Sequence 4590 | AA640687 |
| Sequence 4591 | AA641636 |
| Sequence 4592 | AA641769 |
| Sequence 4593 | AA641810 |
| Sequence 4594 | AA642383 |
| Sequence 4595 | AA643506 |
| Sequence 4596 | AA644237 |
| Sequence 4597 | AA650230 |
| Sequence 4598 | AA652206 |
| Sequence 4599 | AA652478 |
| Sequence 4600 | AA662733 |
| Sequence 4601 | AA664567 |
| Sequence 4602 | AA665507 |
| Sequence 4603 | AA679314 |
| Sequence 4604 | AA679547 |
| Sequence 4605 | AA687216 |
| Sequence 4606 | AA702230 |
| Sequence 4607 | AA706388 |
| Sequence 4608 | AA707607 |
| Sequence 4609 | AA708016 |
| Sequence 4610 | AA708690 |
| Sequence 4611 | AA714481 |
| Sequence 4612 | AA722204 |
| Sequence 4613 | AA724553 |
| Sequence 4614 | AA737949 |
| Sequence 4615 | AA743680 |
| Sequence 4616 | AA744367 |
| Sequence 4617 | AA744771 |
| Sequence 4618 | AA747515 |
| Sequence 4619 | AA747772 |
| Sequence 4620 | AA748847 |
| Sequence 4621 | AA761640 |
| Sequence 4622 | AA764991 |
| Sequence 4623 | AA765884 |
| Sequence 4624 | AA765974 |
| Sequence 4625 | AA766644 |
| Sequence 4626 | AA769055 |
| Sequence 4627 | AA773072 |
| Sequence 4628 | AA773641 |
| Sequence 4629 | AA773787 |
| Sequence 4630 | AA776585 |
| Sequence 4631 | AA779176 |
| Sequence 4632 | AA780591 |
| Sequence 4633 | AA782138 |
| Sequence 4634 | AA782644 |
| Sequence 4635 | AA805865 |
| Sequence 4636 | AA806808 |
| Sequence 4637 | AA811365 |
| Sequence 4638 | AA813108 |
| Sequence 4639 | AA825612 |
| Sequence 4640 | AA828708 |
| Sequence 4641 | AA829473 |
| Sequence 4642 | AA833543 |
| Sequence 4643 | AA836233 |
| Sequence 4644 | AA843242 |
| Sequence 4645 | AA843414 |
| Sequence 4646 | AA843663 |
| Sequence 4647 | AA845340 |
| Sequence 4648 | AA847380 |
| Sequence 4649 | AA854293 |
| Sequence 4650 | AA857509 |
| Sequence 4651 | AA860541 |
| Sequence 4652 | AA860986 |
| Sequence 4653 | AA865873 |
| Sequence 4654 | AA868720 |
| Sequence 4655 | AA873182 |
| Sequence 4656 | AA873291 |
| Sequence 4657 | AA873828 |
| Sequence 4658 | AA876668 |
| Sequence 4659 | AA885770 |
| Sequence 4660 | AA888014 |
| Sequence 4661 | AA890327 |
| Sequence 4662 | AA902127 |
| Sequence 4663 | AA902721 |
| Sequence 4664 | AA902964 |
| Sequence 4665 | AA905139 |
| Sequence 4666 | AA911245 |
| Sequence 4667 | AA916753 |
| Sequence 4668 | AA931228 |
| Sequence 4669 | AA931237 |
| Sequence 4670 | AA931583 |
| Sequence 4671 | AA932114 |
| Sequence 4672 | AA935526 |
| Sequence 4673 | AA935560 |
| Sequence 4674 | AA936262 |
| Sequence 4675 | AA937551 |
| Sequence 4676 | AA938717 |
| Sequence 4677 | AA946873 |
| Sequence 4678 | AA953892 |
| Sequence 4679 | AA954118 |
| Sequence 4880 | AA968756 |
| Sequence 4681 | AA974540 |
| Sequence 4682 | AA975275 |
| Sequence 4683 | AA977172 |
| Sequence 4684 | AA985327 |
| Sequence 4685 | AA987997 |
| Sequence 4686 | AA988125 |
| Sequence 4687 | AA988520 |
| Sequence 4688 | AA995981 |
| Sequence 4689 | AI004252 |
| Sequence 4690 | AI015053 |
| Sequence 4691 | AI017442 |
| Sequence 4692 | AI017996 |
| Sequence 4693 | AI023770 |
| Sequence 4694 | AI024638 |
| Sequence 4695 | AI024799 |
| Sequence 4696 | AI026122 |
| Sequence 4697 | AIO27643 |
| Sequence 4698 | AI032759 |
| Sequence 4699 | AI033912 |
| Sequence 4700 | AI034131 |
| Sequence 4701 | AI038167 |
| Sequence 4702 | AI038415 |
| Sequence 4703 | AI040598 |
| Sequence 4704 | AI041616 |
| Sequence 4705 | AI042153 |
| Sequence 4706 | AI051210 |
| Sequence 4707 | AI051376 |
| Sequence 4708 | AI052453 |
| Sequence 4709 | AI061420 |
| Sequence 4710 | AI064763 |
| Sequence 4711 | AI066518 |
| Sequence 4712 | AI074969 |
| Sequence 4713 | AI075935 |
| Sequence 4714 | AI077439 |
| Sequence 4715 | AI078772 |
| Sequence 4716 | AI080439 |
| Sequence 4717 | AI080476 |
| Sequence 4718 | AI084101 |
| Sequence 4719 | AI085977 |
| Sequence 4720 | AI086377 |
| Sequence 4721 | AI087987 |
| Sequence 4722 | AI088201 |
| Sequence 4723 | AI091190 |
| Sequence 4724 | AI093553 |
| Sequence 4725 | AI125642 |
| Sequence 4726 | AI125688 |
| Sequence 4727 | AI126233 |
| Sequence 4728 | A1126700 |
| Sequence 4729 | AI127167 |
| Sequence 4730 | AI129321 |
| Sequence 4731 | AI141040 |
| Sequence 4732 | AI142257 |
| Sequence 4733 | AI144488 |
| Sequence 4734 | AI148552 |
| Sequence 4735 | AI148676 |
| Sequence 4736 | AI148933 |
| Sequence 4737 | AI159768 |
| Sequence 4738 | AI174675 |
| Sequence 4739 | AI198311 |
| Sequence 4740 | AI198577 |
| Sequence 4741 | AI198986 |
| Sequence 4742 | AI199094 |
| Sequence 4743 | AI199681 |
| Sequence 4744 | AI201483 |
| Sequence 4745 | AI203141 |
| Sequence 4746 | AI203395 |
| Sequence 4747 | AI207650 |
| Sequence 4748 | AI214272 |
| Sequence 4749 | AI214784 |
| Sequence 4750 | AI219278 |
| Sequence 4751 | AI220149 |
| Sequence 4752 | AI221713 |
| Sequence 4753 | AI245327 |
| Sequence 4754 | AI247078 |
| Sequence 4755 | AI248736 |
| Sequence 4756 | AI250092 |
| Sequence 4757 | AI253335 |
| Sequence 4758 | AI253348 |
| Sequence 4759 | AI253379 |
| Sequence 4760 | AI267185 |
| Sequence 4761 | AI267282 |
| Sequence 4762 | AI267454 |
| Sequence 4763 | AI267502 |
| Sequence 4764 | AI267574 |
| Sequence 4765 | AI268724 |
| Sequence 4766 | AI274786 |
| Sequence 4767 | AI278659 |
| Sequence 4768 | AI283489 |
| Sequence 4769 | AI288142 |
| Sequence 4770 | AI289059 |
| Sequence 4771 | AI299945 |
| Sequence 4772 | AI300489 |
| Sequence 4773 | AI301532 |
| Sequence 4774 | AI308800 |
| Sequence 4775 | AI310465 |
| Sequence 4776 | AI312542 |
| Sequence 4777 | AI313387 |
| Sequence 4778 | AI332588 |
| Sequence 4779 | AI333116 |
| Sequence 4780 | AI338338 |
| Sequence 4781 | AI339458 |
| Sequence 4782 | AI343692 |
| Sequence 4783 | AI346021 |
| Sequence 4784 | AI340657 |
| Sequence 4785 | AI357472 |
| Sequence 4786 | AI358723 |
| Sequence 4787 | AI366381 |
| Sequence 4788 | AI366549 |
| Sequence 4789 | AI373289 |
| Sequence 4790 | AI394646 |
| Sequence 4791 | AI400410 |
| Sequence 4792 | AI420172 |
| Sequence 4793 | AI421741 |
| Sequence 4794 | AI423541 |
| Sequence 4795 | AI432462 |
| Sequence 4796 | AI434150 |
| Sequence 4797 | AI452485 |
| Sequence 4798 | AI457964 |
| Sequence 4799 | AI458673 |
| Sequence 4800 | AI460230 |
| Sequence 4801 | AI467858 |
| Sequence 4802 | AI475476 |
| Sequence 4803 | AI478365 |
| Sequence 4804 | AI493851 |
| Sequence 4805 | AI521984 |
| Sequence 4806 | AI524826 |
| Sequence 4807 | AI525102 |
| Sequence 4808 | AI546840 |
| Sequence 4809 | AI557059 |
| Sequence 4810 | AI557112 |
| Sequence 4811 | AI557118 |
| Sequence 4812 | AI557225 |
| Sequence 4813 | AI557243 |
| Sequence 4814 | AI557363 |
| Sequence 4815 | AI557495 |
| Sequence 4816 | AI559299 |
| Sequence 4817 | AI559903 |
| Sequence 4818 | AI560870 |
| Sequence 4819 | AI564487 |
| Sequence 4820 | AI589533 |
| Sequence 4821 | AI608684 |
| Sequence 4822 | AI608968 |
| Sequence 4823 | AI610412 |
| Sequence 4824 | AI619498 |
| Sequence 4825 | AI620796 |
| Sequence 4826 | AI627553 |
| Sequence 4827 | AI635310 |
| Sequence 4828 | AI635658 |
| Sequence 4829 | AI635667 |
| Sequence 4830 | AI659898 |
| Sequence 4831 | AI660356 |
| Sequence 4832 | AI660919 |
| Sequence 4833 | AI671394 |
| Sequence 4834 | AI679619 |
| Sequence 4835 | AI680874 |
| Sequence 4836 | AI682088 |
| Sequence 4837 | AI682937 |
| Sequence 4838 | AI686779 |
| Sequence 4839 | AI693302 |
| Sequence 4840 | AI694199 |
| Sequence 4841 | AI694596 |
| Sequence 4842 | AI696992 |
| Sequence 4843 | AI697849 |
| Sequence 4844 | AI734266 |
| Sequence 4845 | AI740796 |
| Sequence 4846 | AI743852 |
| Sequence 4847 | AI749547 |
| Sequence 4848 | AI753784 |
| Sequence 4849 | AI754013 |
| Sequence 4850 | AI755085 |
| Sequence 4851 | AI762085 |
| Sequence 4852 | AI769392 |
| Sequence 4853 | AI769478 |
| Sequence 4854 | AI791370 |
| Sequence 4855 | AI791906 |
| Sequence 4856 | AI791998 |
| Sequence 4857 | AI801810 |
| Sequence 4858 | AI805379 |
| Sequence 4859 | AI806044 |
| Sequence 4860 | AI813772 |
| Sequence 4861 | AI816102 |
| Sequence 4862 | AI828602 |
| Sequence 4863 | AI829784 |
| Sequence 4864 | AI861997 |
| Sequence 4865 | AI864927 |
| Sequence 4866 | AI871027 |
| Sequence 4867 | AI871745 |
| Sequence 4868 | AI879995 |
| Sequence 4869 | AI884963 |
| Sequence 4870 | AI915264 |
| Sequence 4871 | AI923011 |
| Sequence 4872 | AI935704 |
| Sequence 4873 | AI935710 |
| Sequence 4874 | AI936472 |
| Sequence 4875 | AI948822 |
| Sequence 4876 | AI954585 |
| Sequence 4877 | AI973022 |
| Sequence 4878 | AI973109 |
| Sequence 4879 | AI985122 |
| Sequence 4880 | AL036594 |
| Sequence 4881 | AL036801 |
| Sequence 4882 | AL037456 |
| Sequence 4883 | AL037895 |
| Sequence 4884 | AL038821 |
| Sequence 4885 | AL039175 |
| Sequence 4886 | AL039235 |
| Sequence 4887 | AL039293 |
| Sequence 4888 | AL039349 |
| Sequence 4889 | AL040204 |
| Sequence 4890 | AL043702 |
| Sequence 4891 | AL044985 |
| Sequence 4892 | AL119715 |
| Sequence 4893 | AL120972 |
| Sequence 4894 | AW019951 |
| Sequence 4895 | AW020930 |
| Sequence 4896 | AW021628 |
| Sequence 4897 | AW022112 |
| Sequence 4898 | AW028521 |
| Sequence 4899 | AW052140 |
| Sequence 4900 | AW055209 |
| Sequence 4901 | AW058600 |
| Sequence 4902 | AW080631 |
| Sequence 4903 | AW080845 |
| Sequence 4904 | AW088727 |
| Sequence 4905 | AW131770 |
| Sequence 4906 | AW134672 |
| Sequence 4907 | AW135187 |
| Sequence 4908 | AW135430 |
| Sequence 4909 | AW135666 |
| Sequence 4910 | AW136951 |
| Sequence 4911 | AW138413 |
| Sequence 4912 | AW140049 |
| Sequence 4913 | AW148750 |
| Sequence 4914 | AW150770 |
| Sequence 4915 | AW160363 |
| Sequence 4916 | AW178825 |
| Sequence 4917 | C05816 |
| Sequence 4918 | C06196 |
| Sequence 4919 | C06419 |
| Sequence 4920 | D58694 |
| Sequence 4921 | F07635 |
| Sequence 4922 | F12493 |
| Sequence 4923 | F13272 |
| Sequence 4924 | F22763 |
| Sequence 4925 | F33993 |
| Sequence 4926 | H00742 |
| Sequence 4927 | H02725 |
| Sequence 4928 | H03785 |
| Sequence 4929 | H08511 |
| Sequence 4930 | H09882 |
| Sequence 4931 | H12923 |
| Sequence 4932 | H13008 |
| Sequence 4933 | H13406 |
| Sequence 4934 | H13891 |
| Sequence 4935 | H20744 |
| Sequence 4936 | H25211 |
| Sequence 4937 | H49331 |
| Sequence 4938 | H49828 |
| Sequence 4939 | H54240 |
| Sequence 4940 | H57382 |
| Sequence 4941 | H65423 |
| Sequence 4942 | H69529 |
| Sequence 4943 | H83215 |
| Sequence 4944 | H87419 |
| Sequence 4945 | H95535 |
| Sequence 4946 | H96053 |
| Sequence 4947 | H96527 |
| Sequence 4948 | N34611 |
| Sequence 4949 | N35555 |
| Sequence 4950 | N42986 |
| Sequence 4951 | N47044 |
| Sequence 4952 | N49471 |
| Sequence 4953 | N49534 |
| Sequence 4954 | N52554 |
| Sequence 4955 | N76215 |
| Sequence 4956 | N92498 |
| Sequence 4957 | N94829 |
| Sequence 4958 | N98569 |
| Sequence 4959 | N98672 |
| Sequence 4960 | R14008 |
| Sequence 4961 | R17476 |
| Sequence 4962 | R19498 |
| Sequence 4963 | R19761 |
| Sequence 4964 | R24400 |
| Sequence 4965 | R25577 |
| Sequence 4966 | R33762 |
| Sequence 4967 | R34284 |
| Sequence 4968 | R35797 |
| Sequence 4969 | R49734 |
| Sequence 4970 | R52387 |
| Sequence 4971 | R59846 |
| Sequence 4972 | R61601 |
| Sequence 4973 | R63823 |
| Sequence 4974 | R70570 |
| Sequence 4975 | R75754 |
| Sequence 4976 | T29959 |
| Sequence 4977 | T34760 |
| Sequence 4978 | T48624 |
| Sequence 4979 | T66793 |
| Sequence 4980 | T70123 |
| Sequence 4981 | T72034 |
| Sequence 4982 | T83709 |
| Sequence 4983 | T86219 |
| Sequence 4984 | T89530 |
| Sequence 4985 | T99386 |
| Sequence 4986 | W03386 |
| Sequence 4987 | W04191 |
| Sequence 4988 | W05240 |
| Sequence 4989 | W05402 |
| Sequence 4990 | W06897 |
| Sequence 4991 | W19970 |
| Sequence 4992 | W21894 |
| Sequence 4993 | W22330 |
| Sequence 4994 | W24228 |
| Sequence 4995 | W26413 |
| Sequence 4996 | W30686 |
| Sequence 4997 | W31819 |
| Sequence 4998 | W37614 |
| Sequence 4999 | W44623 |
| Sequence 5000 | W44911 |
| Sequence 5001 | W44954 |
| Sequence 5002 | W56395 |
| Sequence 5003 | W60565 |
| Sequence 5004 | W61042 |
| Sequence 5005 | W72973 |
| Sequence 5006 | W74046 |
| Sequence 5007 | W80673 |
| Sequence 5008 | W96214 |
| Sequence 5009 | Z43304 |
| Sequence 5010 | A06805 |
| Sequence 5011 | A06846 |
| Sequence 5012 | A06919 |
| Sequence 5013 | A16794 |
| Sequence 5014 | AB000888 |
| Sequence 5015 | AB001106 |
| Sequence 5016 | AB002303 |
| Sequence 5017 | AB002330 |
| Sequence 5018 | AB002347 |
| Sequence 5019 | AB002365 |
| Sequence 5020 | AB002369 |
| Sequence 5021 | AB002370 |
| Sequence 5022 | AB002382 |
| Sequence 5023 | AB004047 |
| Sequence 5024 | AB004857 |
| Sequence 5025 | AB007510 |
| Sequence 5026 | AB007896 |
| Sequence 5027 | AB007899 |
| Sequence 5028 | AB007916 |
| Sequence 5029 | AB007931 |
| Sequence 5030 | AB007934 |
| Sequence 5031 | AB007956 |
| Sequence 5032 | AB007969 |
| Sequence 5033 | AB008226 |
| Sequence 5034 | AB009285 |
| Sequence 5035 | AB011004 |
| Sequence 5036 | AB011079 |
| Sequence 5037 | AB011145 |
| Sequence 5038 | AB011159 |
| Sequence 5039 | AB014560 |
| Sequence 5040 | AB015317 |
| Sequence 5041 | AB016068 |
| Sequence 5042 | AB018266 |
| Sequence 5043 | AB018337 |
| Sequence 5044 | AB018340 |
| Sequence 5045 | AB018346 |
| Sequence 5046 | AB019568 |
| Sequence 5047 | AB020631 |
| Sequence 5048 | AB020643 |
| Sequence 5049 | AB020657 |
| Sequence 5050 | AB020694 |
| Sequence 5051 | AB020724 |
| Sequence 5052 | AB021663 |
| Sequence 5053 | AB021981 |
| Sequence 5054 | AB023157 |
| Sequence 5055 | AB023173 |
| Sequence 5056 | AB023177 |
| Sequence 5057 | AB023227 |
| Sequence 5058 | AB023230 |
| Sequence 5059 | AB024334 |
| Sequence 5060 | AB024435 |
| Sequence 5061 | AB026257 |
| Sequence 5062 | AB028624 |
| Sequence 5063 | AB028973 |
| Sequence 5064 | AB028995 |
| Sequence 5065 | AB029036 |
| Sequence 5066 | AB032948 |
| Sequence 5067 | AB032983 |
| Sequence 5068 | AB033042 |
| Sequence 5069 | AB033071 |
| Sequence 5070 | AB033075 |
| Sequence 5071 | AB033076 |
| Sequence 5072 | AB033078 |
| Sequence 5073 | AB033079 |
| Sequence 5074 | AB033080 |
| Sequence 5075 | AB033092 |
| Sequence 5076 | AB033899 |
| Sequence 5077 | AD001528 |
| Sequence 5078 | AF000652 |
| Sequence 5079 | AF000982 |
| Sequence 5080 | AF000983 |
| Sequence 5081 | AF001902 |
| Sequence 5082 | AF002668 |
| Sequence 5083 | AF002697 |
| Sequence 5084 | AF006088 |
| Sequence 5085 | AF007216 |
| Sequence 5086 | AF007791 |
| Sequence 5087 | AF008442 |
| Sequence 5088 | AF010233 |
| Sequence 5089 | AF013759 |
| Sequence 5090 | AF015553 |
| Sequence 5091 | AF016371 |
| Sequence 5092 | AF019214 |
| Sequence 5093 | AF019226 |
| Sequence 5094 | AF020736 |
| Sequence 5095 | AF020797 |
| Sequence 5096 | AF021351 |
| Sequence 5097 | AF026166 |
| Sequence 5098 | AF026246 |
| Sequence 5099 | AF027824 |
| Sequence 5100 | AF028593 |
| Sequence 5101 | AF028832 |
| Sequence 5102 | AF029669 |
| Sequence 5103 | AF030162 |
| Sequence 5104 | AF033095 |
| Sequence 5105 | AF035285 |
| Sequence 5106 | AF035293 |
| Sequence 5107 | AF035316 |
| Sequence 5108 | AF037447 |
| Sequence 5109 | AF038187 |
| Sequence 5110 | AF038451 |
| Sequence 5111 | AF038452 |
| Sequence 5112 | AF038955 |
| Sequence 5113 | AF039023 |
| Sequence 5114 | AF039918 |
| Sequence 5115 | AF042181 |
| Sequence 5116 | AF044953 |
| Sequence 5117 | AF044956 |
| Sequence 5118 | AF047020 |
| Sequence 5119 | AF047470 |
| Sequence 5120 | AF047472 |
| Sequence 5121 | AF048731 |
| Sequence 5122 | AF051334 |
| Sequence 5123 | AF052107 |
| Sequence 5124 | AF052154 |
| Sequence 5125 | AF053535 |
| Sequence 5126 | AF053641 |
| Sequence 5127 | AF054181 |
| Sequence 5128 | AF054186 |
| Sequence 5129 | AF054187 |
| Sequence 5130 | AF054284 |
| Sequence 5131 | AF054838 |
| Sequence 5132 | AF054990 |
| Sequence 5133 | AF057160 |
| Sequence 5134 | AF061258 |
| Sequence 5135 | AF061739 |
| Sequence 5136 | AF062323 |
| Sequence 5137 | AF064257 |
| Sequence 5138 | AF064768 |
| Sequence 5139 | AF068235 |
| Sequence 5140 | AF070523 |
| Sequence 5141 | AF070556 |
| Sequence 5142 | AF070561 |
| Sequence 5143 | AF070621 |
| Sequence 5144 | AF070646 |
| Sequence 5145 | AF070659 |
| Sequence 5146 | AF070661 |
| Sequence 5147 | AF071202 |
| Sequence 5148 | AF073299 |
| Sequence 5149 | AF074606 |
| Sequence 5150 | AF075061 |
| Sequence 5151 | AF077030 |
| Sequence 5152 | AF077202 |
| Sequence 5153 | AF078845 |
| Sequence 5154 | AF078855 |
| Sequence 5155 | AF078860 |
| Sequence 5156 | AF081282 |
| Sequence 5157 | AF083106 |
| Sequence 5158 | AF085224 |
| Sequence 5159 | AF085845 |
| Sequence 5160 | AF085871 |
| Sequence 5161 | AF085896 |
| Sequence 5162 | AF086172 |
| Sequence 5163 | AF086245 |
| Sequence 5164 | AF086336 |
| Sequence 5165 | AF087980 |
| Sequence 5166 | AF091084 |
| Sequence 5167 | AF092092 |
| Sequence 5168 | AF092138 |
| Sequence 5169 | AF097514 |
| Sequence 5170 | AF097725 |
| Sequence 5171 | AF099137 |
| Sequence 5172 | AF100756 |
| Sequence 5173 | AF101074 |
| Sequence 5174 | AF102846 |
| Sequence 5175 | AF103907 |
| Sequence 5176 | AF106681 |
| Sequence 5177 | AF106862 |
| Sequence 5178 | AF109219 |
| Sequence 5179 | AF110377 |
| Sequence 5180 | AF111106 |
| Sequence 5181 | AF118838 |
| Sequence 5182 | AF124141 |
| Sequence 5183 | AF124438 |
| Sequence 5184 | AF124598 |
| Sequence 5185 | AF126245 |
| Sequence 5186 | AF131743 |
| Sequence 5187 | AF131775 |
| Sequence 5188 | AF131831 |
| Sequence 5189 | AF131856 |
| Sequence 5190 | AF131857 |
| Sequence 5191 | AF132000 |
| Sequence 5192 | AF132940 |
| Sequence 5193 | AF132965 |
| Sequence 5194 | AF133123 |
| Sequence 5195 | AF135421 |
| Sequence 5196 | AF144103 |
| Sequence 5197 | AF144566 |
| Sequence 5198 | AF146277 |
| Sequence 5200 | AF151103 |
| Sequence 5201 | AF151109 |
| Sequence 5202 | AF151840 |
| Sequence 5203 | AF151851 |
| Sequence 5204 | AF151871 |
| Sequence 5205 | AF151875 |
| Sequence 5206 | AF151897 |
| Sequence 5207 | AF152097 |
| Sequence 5208 | AF153608 |
| Sequence 5209 | AF155099 |
| Sequence 5210 | AF156098 |
| Sequence 5211 | AF159056 |
| Sequence 5212 | AF159615 |
| Sequence 5213 | AF168956 |
| Sequence 5214 | AF171877 |
| Sequence 5215 | AF174605 |
| Sequence 5216 | AF176555 |
| Sequence 5217 | AF176574 |
| Sequence 5218 | AF176700 |
| Sequence 5219 | AF188746 |
| Sequence 5220 | AF188747 |
| Sequence 5221 | AF201077 |
| Sequence 5222 | AJ001381 |
| Sequence 5223 | AJ001443 |
| Sequence 5224 | AJ002744 |
| Sequence 5225 | AJ002955 |
| Sequence 5226 | AJ004913 |
| Sequence 5227 | AJ005821 |
| Sequence 5228 | AJ007398 |
| Sequence 5229 | AJ011007 |
| Sequence 5230 | AJ012499 |
| Sequence 5231 | AJ223352 |
| Sequence 5232 | AJ223353 |
| Sequence 5233 | AJ224326 |
| Sequence 5234 | AJ249248 |
| Sequence 5235 | AL049266 |
| Sequence 5236 | AL049447 |
| Sequence 5237 | AL049471 |
| Sequence 5238 | AL049705 |
| Sequence 5239 | AL049932 |
| Sequence 5240 | AL049969 |
| Sequence 5241 | AL049996 |
| Sequence 5242 | AL050013 |
| Sequence 5243 | AL050018 |
| Sequence 5244 | AL050035 |
| Sequence 5245 | AL050064 |
| Sequence 5246 | AL050071 |
| Sequence 5247 | AL050161 |
| Sequence 5248 | AL050186 |
| Sequence 5249 | AL050204 |
| Sequence 5250 | AL050268 |
| Sequence 5251 | AL079288 |
| Sequence 5252 | AL079298 |
| Sequence 5253 | AL080059 |
| Sequence 5254 | AL080084 |
| Sequence 5255 | AL080118 |
| Sequence 5256 | AL080192 |
| Sequence 5257 | AL080209 |
| Sequence 5258 | AL080212 |
| Sequence 5259 | AL110126 |
| Sequence 5260 | AL110177 |
| Sequence 5261 | AL110183 |
| Sequence 5262 | AL110207 |
| Sequence 5263 | AL110222 |
| Sequence 5264 | AL110233 |
| Sequence 5265 | AL110241 |
| Sequence 5266 | AL117392 |
| Sequence 5267 | AL117616 |
| Sequence 5268 | AL122121 |
| Sequence 5269 | AL133108 |
| Sequence 5270 | AL133117 |
| Sequence 5271 | D10040 |
| Sequence 5272 | D13286 |
| Sequence 5273 | D13315 |
| Sequence 5274 | D13388 |
| Sequence 5275 | D13627 |
| Sequence 5276 | D13630 |
| Sequence 5277 | D13641 |
| Sequence 5278 | D13866 |
| Sequence 5279 | D13900 |
| Sequence 5280 | D14446 |
| Sequence 5281 | D14530 |
| Sequence 5282 | D14657 |
| Sequence 5283 | D14658 |
| Sequence 5284 | D14696 |
| Sequence 5285 | D14710 |
| Sequence 5286 | D14812 |
| Sequence 5287 | D14878 |
| Sequence 5288 | D16307 |
| Sequence 5289 | D16960 |
| Sequence 5290 | D17039 |
| Sequence 5291 | D17130 |
| Sequence 5292 | D17400 |
| Sequence 5293 | D21163 |
| Sequence 5294 | D21260 |
| Sequence 5295 | D25283 |
| Sequence 5296 | D25542 |
| Sequence 5297 | D26129 |
| Sequence 5298 | D26600 |
| Sequence 5299 | D29640 |
| Sequence 5300 | D29954 |
| Sequence 5301 | D31885 |
| Sequence 5302 | D37991 |
| Sequence 5303 | D38305 |
| Sequence 5304 | D38551 |
| Sequence 5305 | D38552 |
| Sequence 5306 | D42047 |
| Sequence 5307 | D42138 |
| Sequence 5308 | D45887 |
| Sequence 5309 | D49396 |
| Sequence 5310 | D50487 |
| Sequence 5311 | D50525 |
| Sequence 5312 | D50926 |
| Sequence 5313 | D63480 |
| Sequence 5314 | D64015 |
| Sequence 5315 | D78333 |
| Sequence 5316 | D79996 |
| Sequence 5317 | D80005 |
| Sequence 5318 | D80009 |
| Sequence 5319 | D82345 |
| Sequence 5320 | D85777 |
| Sequence 5321 | D86322 |
| Sequence 5322 | D86971 |
| Sequence 5323 | D87443 |
| Sequence 5324 | D87444 |
| Sequence 5325 | D87453 |
| Sequence 5326 | D87742 |
| Sequence 5327 | D87845 |
| Sequence 5328 | D88674 |
| Sequence 5329 | D89053 |
| Sequence 5330 | D89667 |
| Sequence 5331 | D89675 |
| Sequence 5332 | D90373 |
| Sequence 5333 | E01574 |
| Sequence 5334 | E01650 |
| Sequence 5335 | E02628 |
| Sequence 5336 | E03414 |
| Sequence 5337 | E03569 |
| Sequence 5338 | E05957 |
| Sequence 5339 | E06721 |
| Sequence 5340 | J02642 |
| Sequence 5341 | J02888 |
| Sequence 5342 | J02923 |
| Sequence 5343 | J02966 |
| Sequence 5344 | J03007 |
| Sequence 5345 | J03015 |
| Sequence 5346 | J03143 |
| Sequence 5347 | J03248 |
| Sequence 5348 | J03503 |
| Sequence 5349 | J03575 |
| Sequence 5350 | J03592 |
| Sequence 5351 | J03934 |
| Sequence 5352 | J04162 |
| Sequence 5353 | J04183 |
| Sequence 5354 | J05021 |
| Sequence 5355 | J05480 |
| Sequence 5356 | K00558 |
| Sequence 5357 | K01911 |
| Sequence 5358 | L01100 |
| Sequence 5359 | L02426 |
| Sequence 5360 | L05425 |
| Sequence 5361 | L05132 |
| Sequence 5362 | L06133 |
| Sequence 5363 | L06850 |
| Sequence 5364 | L07633 |
| Sequence 5365 | L07758 |
| Sequence 5366 | L08441 |
| Sequence 5367 | L10284 |
| Sequence 5368 | L10678 |
| Sequence 5369 | L11566 |
| Sequence 5370 | L11667 |
| Sequence 5371 | L12686 |
| Sequence 5372 | L13977 |
| Sequence 5373 | L15203 |
| Sequence 5374 | L17128 |
| Sequence 5375 | L29158 |
| Sequence 5376 | L34839 |
| Sequence 5377 | L34840 |
| Sequence 5378 | L35249 |
| Sequence 5379 | L37080 |
| Sequence 5380 | L38608 |
| Sequence 5381 | L38951 |
| Sequence 5382 | L38961 |
| Sequence 5383 | L47647 |
| Sequence 5384 | M10036 |
| Sequence 5385 | M10119 |
| Sequence 5386 | M11058 |
| Sequence 5387 | M11353 |
| Sequence 5388 | M11887 |
| Sequence 5389 | M11949 |
| Sequence 5390 | M15885 |
| Sequence 5391 | M15887 |
| Sequence 5392 | M15990 |
| Sequence 5393 | M16247 |
| Sequence 5394 | M16768 |
| Sequence 5395 | M16804 |
| Sequence 5396 | M16827 |
| Sequence 5397 | M17323 |
| Sequence 5398 | M19961 |
| Sequence 5399 | M20372 |
| Sequence 5400 | M20471 |
| Sequence 5401 | M21154 |
| Sequence 5402 | M21895 |
| Sequence 5403 | M22382 |
| Sequence 5404 | M22590 |
| Sequence 5405 | M23161 |
| Sequence 5406 | M23613 |
| Sequence 5407 | M24194 |
| Sequence 5408 | M24902 |
| Sequence 5409 | M26325 |
| Sequence 5410 | M26481 |
| Sequence 5411 | M26663 |
| Sequence 5412 | M27504 |
| Sequence 5413 | M27691 |
| Sequence 5414 | M28211 |
| Sequence 5415 | M28372 |
| Sequence 5416 | M28526 |
| Sequence 5417 | M29064 |
| Sequence 5418 | M29872 |
| Sequence 5419 | M31630 |
| Sequence 5420 | M33197 |
| Sequence 5421 | M34175 |
| Sequence 5422 | M34181 |
| Sequence 5423 | M34840 |
| Sequence 5424 | M35252 |
| Sequence 5425 | M36634 |
| Sequence 5426 | M37400 |
| Sequence 5427 | M37583 |
| Sequence 5428 | M38690 |
| Sequence 5429 | M55409 |
| Sequence 5430 | M55536 |
| Sequence 5431 | M58581 |
| Sequence 5432 | M59465 |
| Sequence 5433 | M62401 |
| Sequence 5434 | M63573 |
| Sequence 5435 | M65131 |
| Sequence 5436 | M69226 |
| Sequence 5437 | M69238 |
| Sequence 5438 | M74091 |
| Sequence 5439 | M74509 |
| Sequence 5440 | M74777 |
| Sequence 5441 | M81757 |
| Sequence 5442 | M82919 |
| Sequence 5443 | M84443 |
| Sequence 5444 | M86917 |
| Sequence 5445 | M90054 |
| Sequence 5446 | M90309 |
| Sequence 5447 | M90516 |
| Sequence 5448 | M94856 |
| Sequence 5449 | M95724 |
| Sequence 5450 | M96803 |
| Sequence 5451 | S50223 |
| Sequence 5452 | S57235 |
| Sequence 5453 | S59049 |
| Sequence 5454 | S64650 |
| Sequence 5455 | S68015 |
| Sequence 5456 | S69272 |
| Sequence 5457 | S73498 |
| Sequence 5458 | U02680 |
| Sequence 5459 | U05291 |
| Sequence 5480 | U05877 |
| Sequence 5461 | U07151 |
| Sequence 5462 | U07919 |
| Sequence 5463 | U10485 |
| Sequence 5464 | U12465 |
| Sequence 5465 | U12789 |
| Sequence 5466 | U13616 |
| Sequence 5467 | U14750 |
| Sequence 5468 | U14967 |
| Sequence 5469 | U14968 |
| Sequence 5470 | U14970 |
| Sequence 5471 | U15008 |
| Sequence 5472 | U17040 |
| Sequence 5473 | U18197 |
| Sequence 5474 | U18914 |
| Sequence 5475 | U19177 |
| Sequence 5476 | U25182 |
| Sequence 5477 | U25789 |
| Sequence 5478 | U27143 |
| Sequence 5479 | U28249 |
| Sequence 5480 | U30826 |
| Sequence 5481 | U31383 |
| Sequence 5482 | U33760 |
| Sequence 5483 | U33821 |
| Sequence 5484 | U34252 |
| Sequence 5485 | U37230 |
| Sequence 5486 | U37518 |
| Sequence 5487 | U39400 |
| Sequence 5488 | U40272 |
| Sequence 5489 | U42404 |
| Sequence 5490 | U43899 |
| Sequence 5491 | U47414 |
| Sequence 5492 | U54558 |
| Sequence 5493 | U57052 |
| Sequence 5494 | U57057 |
| Sequence 5495 | U58855 |
| Sequence 5496 | U59919 |
| Sequence 5497 | U60205 |
| Sequence 5498 | U61232 |
| Sequence 5499 | U62740 |
| Sequence 5500 | U63542 |
| Sequence 5501 | U68140 |
| Sequence 5502 | U68758 |
| Sequence 5503 | U70063 |
| Sequence 5504 | U70322 |
| Sequence 5505 | U70451 |
| Sequence 5506 | U72761 |
| Sequence 5507 | U75272 |
| Sequence 5508 | U75679 |
| Sequence 5509 | U78575 |
| Sequence 5510 | U80034 |
| Sequence 5511 | U83115 |
| Sequence 5512 | U85658 |
| Sequence 5513 | U87166 |
| Sequence 5514 | U90915 |
| Sequence 5515 | U96759 |
| Sequence 5516 | V00478 |
| Sequence 5517 | X02530 |
| Sequence 5518 | X04098 |
| Sequence 5519 | X05332 |
| Sequence 5520 | X05345 |
| Sequence 5521 | X06272 |
| Sequence 5522 | X06617 |
| Sequence 5523 | X12433 |
| Sequence 5524 | X12791 |
| Sequence 5525 | X13425 |
| Sequence 5526 | X13482 |
| Sequence 5527 | X13585 |
| Sequence 5528 | X13839 |
| Sequence 5529 | X15187 |
| Sequence 5530 | X15729 |
| Sequence 5531 | X52882 |
| Sequence 5532 | X53605 |
| Sequence 5533 | X53793 |
| Sequence 5534 | X54199 |
| Sequence 5535 | X54473 |
| Sequence 5536 | X56932 |
| Sequence 5537 | X61970 |
| Sequence 5538 | X62534 |
| Sequence 5539 | X63753 |
| Sequence 5540 | X64330 |
| Sequence 5541 | X64838 |
| Sequence 5542 | X66276 |
| Sequence 5543 | X66785 |
| Sequence 5544 | X67098 |
| Sequence 5545 | X73114 |
| Sequence 5546 | X73608 |
| Sequence 5547 | X76770 |
| Sequence 5548 | X76771 |
| Sequence 5549 | X77196 |
| Sequence 5550 | X78627 |
| Sequence 5551 | X79201 |
| Sequence 5552 | X81198 |
| Sequence 5553 | X81695 |
| Sequence 5554 | X82157 |
| Sequence 5555 | X84908 |
| Sequence 5556 | X85337 |
| Sequence 5557 | X85372 |
| Sequence 5558 | X85373 |
| Sequence 5559 | X86098 |
| Sequence 5560 | X87176 |
| Sequence 5561 | X87949 |
| Sequence 5562 | X94323 |
| Sequence 5563 | X95384 |
| Sequence 5564 | X97065 |
| Sequence 5565 | X97675 |
| Sequence 5566 | X98263 |
| Sequence 5567 | Y00282 |
| Sequence 5568 | Y00345 |
| Sequence 5569 | Y08991 |
| Sequence 5570 | Y09615 |
| Sequence 5571 | Y09836 |
| Sequence 5572 | Y12065 |
| Sequence 5573 | Y12860 |
| Sequence 5574 | Y13286 |
| Sequence 5575 | Y15286 |
| Sequence 5576 | Y16132 |
| Sequence 5577 | Y16241 |
| Sequence 5578 | Y16414 |
| Sequence 5579 | Y18314 |
| Sequence 5580 | Z21507 |
| Sequence 5581 | Z22658 |
| Sequence 5582 | Z36531 |
| Sequence 5583 | Z37986 |
| Sequence 5584 | Z47087 |
| Sequence 5585 | Z47553 |
| Sequence 5586 | N91825 |
| Sequence 5587 | Q92813 |
| Sequence 5588 | T47520 |
| Sequence 5589 | T59274 |
| Sequence 5590 | T84934 |
| Sequence 5591 | V04715 |
| Sequence 5592 | V07572 |
| Sequence 5593 | V44867 |
| Sequence 5594 | V58694 |
| Sequence 5595 | V62427 |
| Sequence 5596 | V63172 |
| Sequence 5597 | V70354 |
| Sequence 5598 | X03841 |
| Sequence 5599 | X24060 |
| Sequence 5600 | X26850 |
| Sequence 5601 | X27262 |
| Sequence 5602 | X29296 |
| Sequence 5603 | X37405 |
| Sequence 5604 | X37486 |
| Sequence 5605 | X40518 |
| Sequence 5606 | X41115 |
| Sequence 5607 | X52268 |
| Sequence 5608 | X87388 |
| Sequence 5609 | X89852 |
| Sequence 5610 | Z00428 |
| Sequence 5611 | Z13254 |
| Sequence 5612 | Z14392 |
| Sequence 5613 | Z15132 |
| Sequence 5614 | AC29430 |
| Sequence 5615 | A06919 |
| Sequence 5616 | A06925 |
| Sequence 5617 | AB002370 |
| Sequence 5618 | AB002375 |
| Sequence 5619 | AB006534 |
| Sequence 5620 | AB007510 |
| Sequence 5621 | AB007899 |
| Sequence 5622 | AB011079 |
| Sequence 5623 | AB014458 |
| Sequence 5624 | AB015639 |
| Sequence 5625 | AB016068 |
| Sequence 5626 | AB018276 |
| Sequence 5627 | AB018313 |
| Sequence 5628 | AB020646 |
| Sequence 5629 | AB020693 |
| Sequence 5630 | AB020694 |
| Sequence 5631 | AB023173 |
| Sequence 5632 | AB023182 |
| Sequence 5633 | AB023227 |
| Sequence 5634 | AB033114 |
| Sequence 5635 | AF007791 |
| Sequence 5636 | AF015040 |
| Sequence 5637 | AF016371 |
| Sequence 5638 | AF019632 |
| Sequence 5639 | AF020797 |
| Sequence 5640 | AF044195 |
| Sequence 5641 | AF047020 |
| Sequence 5642 | AF052642 |
| Sequence 5643 | AF061258 |
| Sequence 5644 | AF067008 |
| Sequence 5645 | AF070525 |
| Sequence 5646 | AF070561 |
| Sequence 5647 | AF071202 |
| Sequence 5648 | AF077202 |
| Sequence 5649 | AF078854 |
| Sequence 5650 | AF085845 |
| Sequence 5651 | AF093097 |
| Sequence 5652 | AF100756 |
| Sequence 5653 | AF100759 |
| Sequence 5654 | AF103907 |
| Sequence 5655 | AF104914 |
| Sequence 5656 | AF126008 |
| Sequence 5657 | AF126736 |
| Sequence 5658 | AF131856 |
| Sequence 5659 | AF132000 |
| Sequence 5660 | AF146277 |
| Sequence 5661 | AF151103 |
| Sequence 5662 | AF151893 |
| Sequence 5663 | AF153612 |
| Sequence 5664 | AF159056 |
| Sequence 5665 | AF188746 |
| Sequence 5666 | AF191298 |
| Sequence 5667 | AJ002744 |
| Sequence 5668 | AJ132637 |
| Sequence 5669 | AJ250042 |
| Sequence 5670 | AL049932 |
| Sequence 5671 | AL117499 |
| Sequence 5672 | D30655 |
| Sequence 5673 | D38551 |
| Sequence 5674 | D42044 |
| Sequence 5675 | D49396 |
| Sequence 5676 | D50371 |
| Sequence 5677 | D50525 |
| Sequence 5678 | E02628 |
| Sequence 5679 | J02959 |
| Sequence 5680 | J03007 |
| Sequence 5681 | K01144 |
| Sequence 5682 | K01911 |
| Sequence 5683 | L09235 |
| Sequence 5684 | L10678 |
| Sequence 5685 | L12136 |
| Sequence 5686 | L15203 |
| Sequence 5687 | L34839 |
| Sequence 5688 | M10119 |
| Sequence 5689 | M13231 |
| Sequence 5690 | M15885 |
| Sequence 5691 | M16553 |
| Sequence 5692 | M16768 |
| Sequence 5693 | M17885 |
| Sequence 5694 | M24902 |
| Sequence 5695 | M26663 |
| Sequence 5696 | M74777 |
| Sequence 5697 | M97935 |
| Sequence 5698 | S69272 |
| Sequence 5699 | S69738 |
| Sequence 5700 | S75755 |
| Sequence 5701 | U07919 |
| Sequence 5702 | U21936 |
| Sequence 5703 | U40272 |
| Sequence 5704 | U42404 |
| Sequence 5705 | U79751 |
| Sequence 5706 | U96759 |
| Sequence 5707 | X05606 |
| Sequence 5708 | X06617 |
| Sequence 5709 | X12791 |
| Sequence 5710 | X15187 |
| Sequence 5711 | X52174 |
| Sequence 5712 | X78627 |
| Sequence 5713 | X85372 |
| Sequence 5714 | X92845 |
| Sequence 5715 | X94323 |
| Sequence 5716 | Y00345 |
| Sequence 5717 | Z11793 |
| Sequence 5718 | AA026715 |
| Sequence 5719 | AA033901 |
| Sequence 5720 | AA035189 |
| Sequence 5721 | AA043802 |
| Sequence 5722 | AA081708 |
| Sequence 5723 | AA083482 |
| Sequence 5724 | AA092533 |
| Sequence 5725 | AA094656 |
| Sequence 5726 | AA100486 |
| Sequence 5727 | AA128739 |
| Sequence 5728 | AA133307 |
| Sequence 5729 | AA135001 |
| Sequence 5730 | AA148729 |
| Sequence 5731 | AA156369 |
| Sequence 5732 | AA156971 |
| Sequence 5733 | AA171510 |
| Sequence 5734 | AA172266 |
| Sequence 5735 | AA179315 |
| Sequence 5736 | AA181102 |
| Sequence 5737 | AA182502 |
| Sequence 5738 | AA189150 |
| Sequence 5739 | AA203252 |
| Sequence 5740 | AA205496 |
| Sequence 5741 | AA224124 |
| Sequence 5742 | AA225115 |
| Sequence 5743 | AA236774 |
| Sequence 5744 | AA249195 |
| Sequence 5745 | AA258750 |
| Sequence 5746 | AA262783 |
| Sequence 5747 | AA278475 |
| Sequence 5748 | AA291041 |
| Sequence 5749 | AA292642 |
| Sequence 5750 | AA298475 |
| Sequence 5751 | AA299404 |
| Sequence 5752 | AA305599 |
| Sequence 5753 | AA306883 |
| Sequence 5754 | AA321319 |
| Sequence 5755 | AA333358 |
| Sequence 5756 | AA339960 |
| Sequence 5757 | AA370352 |
| Sequence 5758 | AA370501 |
| Sequence 5759 | AA371265 |
| Sequence 5760 | AA393313 |
| Sequence 5761 | AA400692 |
| Sequence 5762 | AA422007 |
| Sequence 5763 | AA433848 |
| Sequence 5764 | AA441885 |
| Sequence 5765 | AA447401 |
| Sequence 5766 | AA454557 |
| Sequence 5767 | AA455668 |
| Sequence 5768 | AA468839 |
| Sequence 5769 | AA478518 |
| Sequence 5770 | AA479342 |
| Sequence 5771 | AA479860 |
| Sequence 5772 | AA485217 |
| Sequence 5773 | AA485935 |
| Sequence 5774 | AA489433 |
| Sequence 5775 | AA491041 |
| Sequence 5776 | AA491249 |
| Sequence 5777 | AA505076 |
| Sequence 5778 | AA506953 |
| Sequence 5779 | AA507804 |
| Sequence 5780 | AA515143 |
| Sequence 5781 | AA533057 |
| Sequence 5782 | AA541587 |
| Sequence 5783 | AA573762 |
| Sequence 5784 | AA578209 |
| Sequence 5785 | AA578773 |
| Sequence 5786 | AA581222 |
| Sequence 5787 | AA602522 |
| Sequence 5788 | AA602957 |
| Sequence 5789 | AA609384 |
| Sequence 5790 | AA644495 |
| Sequence 5791 | AA652478 |
| Sequence 5792 | AA659693 |
| Sequence 5793 | AA683246 |
| Sequence 5794 | AA687216 |
| Sequence 5795 | AA708003 |
| Sequence 5796 | AA759202 |
| Sequence 5797 | AA760894 |
| Sequence 5798 | AA779728 |
| Sequence 5799 | AA806808 |
| Sequence 5800 | AA831278 |
| Sequence 5801 | AA861930 |
| Sequence 5802 | AA878195 |
| Sequence 5803 | AA913138 |
| Sequence 5804 | AA935560 |
| Sequence 5805 | AA947278 |
| Sequence 5806 | AA953892 |
| Sequence 5807 | AA974999 |
| Sequence 5808 | AA975275 |
| Sequence 5809 | AA984134 |
| Sequence 5810 | AI018034 |
| Sequence 5811 | AI018625 |
| Sequence 5812 | AI049995 |
| Sequence 5813 | AI084651 |
| Sequence 5814 | AI090226 |
| Sequence 5815 | AI125642 |
| Sequence 5816 | AI126233 |
| Sequence 5817 | AI133406 |
| Sequence 5818 | A1133727 |
| Sequence 5819 | AI200578 |
| Sequence 5820 | AI282318 |
| Sequence 5821 | AI300489 |
| Sequence 5822 | AI309401 |
| Sequence 5823 | AI422907 |
| Sequence 5824 | AI453358 |
| Sequence 5825 | AI460230 |
| Sequence 5826 | AI479021 |
| Sequence 5827 | AI480214 |
| Sequence 5828 | AI497759 |
| Sequence 5829 | AI557112 |
| Sequence 5830 | AI581840 |
| Sequence 5831 | AI632108 |
| Sequence 5832 | AI678832 |
| Sequence 5833 | AI734266 |
| Sequence 5834 | AI755085 |
| Sequence 5835 | AI796571 |
| Sequence 5836 | AI815454 |
| Sequence 5837 | AI864040 |
| Sequence 5838 | AI884963 |
| Sequence 5839 | AL037316 |
| Sequence 5840 | AL037732 |
| Sequence 5841 | AL039293 |
| Sequence 5842 | AL039599 |
| Sequence 5843 | AW014145 |
| Sequence 5844 | AW020934 |
| Sequence 5845 | AW085924 |
| Sequence 5846 | AW135430 |
| Sequence 5847 | AW173228 |
| Sequence 5848 | D58694 |
| Sequence 5849 | H51866 |
| Sequence 5850 | N40017 |
| Sequence 5851 | R22748 |
| Sequence 5852 | R24400 |
| Sequence 5853 | R49734 |
| Sequence 5854 | R61436 |
| Sequence 5855 | R78584 |
| Sequence 5856 | T81995 |
| Sequence 5857 | T99386 |
| Sequence 5858 | W24708 |
| Sequence 5859 | T59274 |
| Sequence 5860 | X06834 |
| Sequence 5861 | X24060 |
| Sequence 5862 | X27262 |
| Sequence 5863 | X52268 |
| Sequence 5864 | Z13254 |
| Sequence 5865 | AC36079 |
| Sequence 5866 | AC003991 |
| Sequence 5867 | AC007275 |
| Sequence 5868 | AP000502 |
| Sequence 5869 | AC007239 |
| Sequence 5870 | AC000065 |
| Sequence 5871 | AC005881 |
| Sequence 5872 | AC004982 |
| Sequence 5873 | AC003991 |
| Sequence 5874 | AC004022 |
| Sequence 5875 | AC004706 |
| Sequence 5876 | U73642 |
| Sequence 5877 | AC005880 |
| Sequence 5878 | AF178030 |
| Sequence 5879 | AF072847 |
| Sequence 5880 | AL121790 |
| Sequence 5881 | AC007279 |
| Sequence 5882 | AA001116 |
| Sequence 5883 | AA001734 |
| Sequence 5884 | AA001794 |
| Sequence 5885 | AA004802 |
| Sequence 5886 | AA009818 |
| Sequence 5887 | AA009878 |
| Sequence 5888 | AA010174 |
| Sequence 5889 | AA010682 |
| Sequence 5890 | AA010981 |
| Sequence 5891 | AA011070 |
| Sequence 5892 | AA013090 |
| Sequence 5893 | AA017474 |
| Sequence 5894 | AA018591 |
| Sequence 5895 | AA019781 |
| Sequence 5896 | AA019874 |
| Sequence 5897 | AA022636 |
| Sequence 5898 | AA022937 |
| Sequence 5899 | AA024796 |
| Sequence 5900 | AA025089 |
| Sequence 5901 | AA025968 |
| Sequence 5902 | AA026983 |
| Sequence 5903 | AA029039 |
| Sequence 5904 | AA029070 |
| Sequence 5905 | AA029143 |
| Sequence 5906 | AA029276 |
| Sequence 5907 | AA029676 |
| Sequence 5908 | AA032220 |
| Sequence 5909 | AA033651 |
| Sequence 5910 | AA033907 |
| Sequence 5911 | AA034979 |
| Sequence 5912 | AA035790 |
| Sequence 5913 | AA037210 |
| Sequence 5914 | AA037483 |
| Sequence 5915 | AA037886 |
| Sequence 5916 | AA039381 |
| Sequence 5917 | AA040810 |
| Sequence 5918 | AA043137 |
| Sequence 5919 | AA043159 |
| Sequence 5920 | AA043415 |
| Sequence 5921 | AA044239 |
| Sequence 5922 | AA044941 |
| Sequence 5923 | AA045147 |
| Sequence 5924 | AA045410 |
| Sequence 5925 | AA045803 |
| Sequence 5926 | AA046848 |
| Sequence 5927 | AA046973 |
| Sequence 5928 | AA046981 |
| Sequence 5929 | AA053909 |
| Sequence 5930 | AA054492 |
| Sequence 5931 | AA056060 |
| Sequence 5932 | AA056358 |
| Sequence 5933 | AA056365 |
| Sequence 5934 | AA056396 |
| Sequence 5935 | AA056528 |
| Sequence 5936 | AA057071 |
| Sequence 5937 | AA057290 |
| Sequence 5938 | AA058398 |
| Sequence 5939 | AA058899 |
| Sequence 5940 | AA058902 |
| Sequence 5941 | AA062583 |
| Sequence 5942 | AA062994 |
| Sequence 5943 | AA063247 |
| Sequence 5944 | AA069367 |
| Sequence 5945 | AA069561 |
| Sequence 5946 | AA071165 |
| Sequence 5947 | AA074086 |
| Sequence 5948 | AA074183 |
| Sequence 5949 | AA074445 |
| Sequence 5950 | AA074509 |
| Sequence 5951 | AA075460 |
| Sequence 5952 | AA076295 |
| Sequence 5953 | AA081007 |
| Sequence 5954 | AA081355 |
| Sequence 5955 | AA082209 |
| Sequence 5956 | AA082572 |
| Sequence 5957 | AA083410 |
| Sequence 5958 | AA083411 |
| Sequence 5959 | AA083482 |
| Sequence 5960 | AA083510 |
| Sequence 5961 | AA083772 |
| Sequence 5962 | AA083836 |
| Sequence 5963 | AA084221 |
| Sequence 5964 | AA084698 |
| Sequence 5965 | AA085480 |
| Sequence 5966 | AA086007 |
| Sequence 5967 | AA088679 |
| Sequence 5968 | AA088758 |
| Sequence 5969 | AA090106 |
| Sequence 5970 | AA090203 |
| Sequence 5971 | AA090548 |
| Sequence 5972 | AA091275 |
| Sequence 5973 | AA092060 |
| Sequence 5974 | AA092343 |
| Sequence 5975 | AA092532 |
| Sequence 5976 | AA092533 |
| Sequence 5977 | AA093935 |
| Sequence 5978 | AA094331 |
| Sequence 5979 | AA094656 |
| Sequence 5980 | AA099110 |
| Sequence 5981 | AA099976 |
| Sequence 5982 | AA100852 |
| Sequence 5983 | AA101777 |
| Sequence 5984 | AA112626 |
| Sequence 5985 | AA113403 |
| Sequence 5986 | AA115021 |
| Sequence 5987 | AA115297 |
| Sequence 5988 | AA115838 |
| Sequence 5989 | AA115933 |
| Sequence 5990 | AA121419 |
| Sequence 5991 | AA126029 |
| Sequence 5992 | AA126742 |
| Sequence 5993 | AA128114 |
| Sequence 5994 | AA128647 |
| Sequence 5995 | AA128668 |
| Sequence 5996 | AA129772 |
| Sequence 5997 | AA129964 |
| Sequence 5998 | AA130207 |
| Sequence 5999 | AA130349 |
| Sequence 6000 | AA131227 |
| Sequence 6001 | AA131252 |
| Sequence 6002 | AA131284 |
| Sequence 6003 | AA131793 |
| Sequence 6004 | AA132510 |
| Sequence 6005 | AA132792 |
| Sequence 6006 | AA133068 |
| Sequence 6007 | AA133268 |
| Sequence 6008 | AA133788 |
| Sequence 6009 | AA134701 |
| Sequence 6010 | AA135870 |
| Sequence 6011 | AA137165 |
| Sequence 6012 | AA143242 |
| Sequence 6013 | AA143523 |
| Sequence 6014 | AA143548 |
| Sequence 6015 | AA146582 |
| Sequence 6016 | AA146900 |
| Sequence 6017 | AA147608 |
| Sequence 6018 | AA147812 |
| Sequence 6019 | AA148019 |
| Sequence 6020 | AA148269 |
| Sequence 6021 | AA148569 |
| Sequence 6022 | AA149490 |
| Sequence 6023 | AA149543 |
| Sequence 6024 | AA149551 |
| Sequence 6025 | AA149579 |
| Sequence 6026 | AA149852 |
| Sequence 6027 | AA150480 |
| Sequence 6028 | AA150650 |
| Sequence 6029 | AA150780 |
| Sequence 6030 | AA150891 |
| Sequence 6031 | AA151596 |
| Sequence 6032 | AA152073 |
| Sequence 6033 | AA152266 |
| Sequence 6034 | AA152440 |
| Sequence 6035 | AA155828 |
| Sequence 6036 | AA155865 |
| Sequence 6037 | AA156369 |
| Sequence 6038 | AA157632 |
| Sequence 6039 | AA159272 |
| Sequence 6040 | AA159647 |
| Sequence 6041 | AA159771 |
| Sequence 6042 | AA160677 |
| Sequence 6043 | AA165027 |
| Sequence 6044 | AA165082 |
| Sequence 6045 | AA165165 |
| Sequence 6046 | AA165632 |
| Sequence 6047 | AA166707 |
| Sequence 6048 | AA167399 |
| Sequence 6049 | AA169507 |
| Sequence 6050 | AA171510 |
| Sequence 6051 | AA173391 |
| Sequence 6052 | AA173559 |
| Sequence 6053 | AA177050 |
| Sequence 6054 | AA179187 |
| Sequence 6055 | AA179462 |
| Sequence 6056 | AA179487 |
| Sequence 6057 | AA180137 |
| Sequence 6058 | AA180154 |
| Sequence 6059 | AA181618 |
| Sequence 6060 | AA182457 |
| Sequence 6061 | AA186538 |
| Sequence 6062 | AA187928 |
| Sequence 6063 | AA187998 |
| Sequence 6064 | AA188052 |
| Sequence 6065 | AA188312 |
| Sequence 6066 | AA188396 |
| Sequence 6067 | AA188875 |
| Sequence 6068 | AA189150 |
| Sequence 6069 | AA190529 |
| Sequence 6070 | AA190661 |
| Sequence 6071 | AA190843 |
| Sequence 6072 | AA190890 |
| Sequence 6073 | AA191045 |
| Sequence 6074 | AA191719 |
| Sequence 6075 | AA191721 |
| Sequence 6076 | AA192597 |
| Sequence 6077 | AA194062 |
| Sequence 6078 | AA194988 |
| Sequence 6079 | AA196001 |
| Sequence 6080 | AA196392 |
| Sequence 6081 | AA196978 |
| Sequence 6082 | AA199658 |
| Sequence 6083 | AA203172 |
| Sequence 6084 | AA203206 |
| Sequence 6085 | AA203285 |
| Sequence 6086 | AA203637 |
| Sequence 6087 | AA205033 |
| Sequence 6088 | AA205647 |
| Sequence 6089 | AA205857 |
| Sequence 6090 | AA206769 |
| Sequence 6091 | AA210760 |
| Sequence 6092 | AA214668 |
| Sequence 6093 | AA214688 |
| Sequence 6094 | AA218788 |
| Sequence 6095 | AA223249 |
| Sequence 6096 | AA223433 |
| Sequence 6097 | AA223956 |
| Sequence 6098 | AA224124 |
| Sequence 6099 | AA224994 |
| Sequence 6100 | AA225115 |
| Sequence 6101 | AA225294 |
| Sequence 6102 | AA225488 |
| Sequence 6103 | AA226171 |
| Sequence 6104 | AA226234 |
| Sequence 6105 | AA226321 |
| Sequence 6106 | AA228273 |
| Sequence 6107 | AA228431 |
| Sequence 6108 | AA228822 |
| Sequence 6109 | AA228940 |
| Sequence 6110 | AA229495 |
| Sequence 6111 | AA229611 |
| Sequence 6112 | AA232691 |
| Sequence 6113 | AA232700 |
| Sequence 6114 | AA233073 |
| Sequence 6115 | AA233088 |
| Sequence 6116 | AA233131 |
| Sequence 6117 | AA234073 |
| Sequence 6118 | AA234092 |
| Sequence 6119 | AA234990 |
| Sequence 6120 | AA235914 |
| Sequence 6121 | AA236320 |
| Sequence 6122 | AA242891 |
| Sequence 6123 | AA243143 |
| Sequence 6124 | AA243696 |
| Sequence 6125 | AA244003 |
| Sequence 6126 | AA244116 |
| Sequence 6127 | AA244207 |
| Sequence 6128 | AA250748 |
| Sequence 6129 | AA251178 |
| Sequence 6130 | AA251578 |
| Sequence 6131 | AA251644 |
| Sequence 6132 | AA252849 |
| Sequence 6133 | AA253453 |
| Sequence 6134 | AA255761 |
| Sequence 6135 | AA256330 |
| Sequence 6136 | AA257973 |
| Sequence 6137 | AA258357 |
| Sequence 6138 | AA258513 |
| Sequence 6139 | AA261990 |
| Sequence 6140 | AA262513 |
| Sequence 6141 | AA263153 |
| Sequence 6142 | AA278520 |
| Sequence 6143 | AA279517 |
| Sequence 6l44 | AA280917 |
| Sequence 6145 | AA284615 |
| Sequence 6146 | AA286953 |
| Sequence 6147 | AA287511 |
| Sequence 6148 | AA291325 |
| Sequence 6149 | AA291363 |
| Sequence 6150 | AA291602 |
| Sequence 6151 | AA291764 |
| Sequence 6152 | AA292638 |
| Sequence 6153 | AA293028 |
| Sequence 6154 | AA293629 |
| Sequence 6155 | AA295182 |
| Sequence 6156 | AA295788 |
| Sequence 6157 | AA296846 |
| Sequence 6158 | AA297941 |
| Sequence 6159 | AA298085 |
| Sequence 6160 | AA298104 |
| Sequence 6161 | AA298294 |
| Sequence 6162 | AA298464 |
| Sequence 6163 | AA298489 |
| Sequence 6164 | AA300065 |
| Sequence 6165 | AA302457 |
| Sequence 6166 | AA305358 |
| Sequence 6167 | AA305627 |
| Sequence 6168 | AA305824 |
| Sequence 6169 | AA305895 |
| Sequence 6170 | AA305909 |
| Sequence 6171 | AA306264 |
| Sequence 6172 | AA306413 |
| Sequence 6173 | AA306480 |
| Sequence 6174 | AA306573 |
| Sequence 6175 | AA306812 |
| Sequence 6176 | AA306956 |
| Sequence 6177 | AA306982 |
| Sequence 6178 | AA306983 |
| Sequence 6179 | AA307513 |
| Sequence 6180 | AA307697 |
| Sequence 6181 | AA308091 |
| Sequence 6182 | AA309909 |
| Sequence 6183 | AA310173 |
| Sequence 6184 | AA310807 |
| Sequence 6185 | AA311028 |
| Sequence 6186 | AA311905 |
| Sequence 6187 | AA311978 |
| Sequence 6188 | AA312059 |
| Sequence 6189 | AA312591 |
| Sequence 6190 | AA312756 |
| Sequence 6191 | AA313057 |
| Sequence 6192 | AA313244 |
| Sequence 6193 | AA313653 |
| Sequence 6194 | AA313979 |
| Sequence 6195 | AA314659 |
| Sequence 6196 | AA314810 |
| Sequence 6197 | AA315016 |
| Sequence 6198 | AA316411 |
| Sequence 6199 | AA317068 |
| Sequence 6200 | AA317496 |
| Sequence 6201 | AA317976 |
| Sequence 6202 | AA320004 |
| Sequence 6203 | AA320723 |
| Sequence 6204 | AA322540 |
| Sequence 6205 | AA324478 |
| Sequence 6206 | AA326306 |
| Sequence 6207 | AA327105 |
| Sequence 6208 | AA327469 |
| Sequence 6209 | AA328770 |
| Sequence 6210 | AA329242 |
| Sequence 6211 | AA329770 |
| Sequence 6212 | AA329937 |
| Sequence 6213 | AA330123 |
| Sequence 6214 | AA332083 |
| Sequence 6215 | AA332593 |
| Sequence 6216 | AA332716 |
| Sequence 6217 | AA333090 |
| Sequence 6218 | AA333526 |
| Sequence 6219 | AA333765 |
| Sequence 6220 | AA334344 |
| Sequence 6221 | AA338215 |
| Sequence 6222 | AA340635 |
| Sequence 6223 | AA344625 |
| Sequence 6224 | AA349232 |
| Sequence 6225 | AA351297 |
| Sequence 6226 | AA352938 |
| Sequence 6227 | AA354872 |
| Sequence 6228 | AA358790 |
| Sequence 6229 | AA359838 |
| Sequence 6230 | AA360190 |
| Sequence 6231 | AA360310 |
| Sequence 6232 | AA364171 |
| Sequence 6233 | AA364844 |
| Sequence 6234 | AA365620 |
| Sequence 6235 | AA366995 |
| Sequence 6236 | AA367471 |
| Sequence 6237 | AA370110 |
| Sequence 6238 | AA370222 |
| Sequence 6239 | AA370352 |
| Sequence 6240 | AA370507 |
| Sequence 6241 | AA370620 |
| Sequence 6242 | AA371017 |
| Sequence 6243 | AA371157 |
| Sequence 6244 | AA371265 |
| Sequence 6245 | AA374020 |
| Sequence 6246 | AA374198 |
| Sequence 6247 | AA374814 |
| Sequence 6248 | AA375927 |
| Sequence 6249 | AA376034 |
| Sequence 6250 | AA378933 |
| Sequence 6251 | AA379448 |
| Sequence 6252 | AA397832 |
| Sequence 6253 | AA397953 |
| Sequence 6254 | AA398732 |
| Sequence 6255 | AA399275 |
| Sequence 6256 | AA400696 |
| Sequence 6257 | AA401206 |
| Sequence 6258 | AA402115 |
| Sequence 6259 | AA403120 |
| Sequence 6260 | AA403121 |
| Sequence 6261 | AA403188 |
| Sequence 6262 | AA404444 |
| Sequence 6263 | AA416736 |
| Sequence 6264 | AA417876 |
| Sequence 6265 | AA418628 |
| Sequence 6266 | AA419263 |
| Sequence 6267 | AA419554 |
| Sequence 6268 | AA420571 |
| Sequence 6269 | AA420633 |
| Sequence 6270 | AA420705 |
| Sequence 6271 | AA421682 |
| Sequence 6272 | AA421850 |
| Sequence 6273 | AA422060 |
| Sequence 6274 | AA423801 |
| Sequence 6275 | AA425407 |
| Sequence 6276 | AA425526 |
| Sequence 6277 | AA426578 |
| Sequence 6278 | AA427866 |
| Sequence 6279 | AA428008 |
| Sequence 6280 | AA428084 |
| Sequence 6281 | AA429014 |
| Sequence 6282 | AA429293 |
| Sequence 6283 | AA429493 |
| Sequence 6284 | AA430110 |
| Sequence 6285 | AA431677 |
| Sequence 6286 | AA432103 |
| Sequence 6287 | AA433869 |
| Sequence 6288 | AA433988 |
| Sequence 6289 | AA435565 |
| Sequence 6290 | AA437224 |
| Sequence 6291 | AA442184 |
| Sequence 6292 | AA442976 |
| Sequence 6293 | AA443024 |
| Sequence 6294 | AA443082 |
| Sequence 6295 | AA443242 |
| Sequence 6296 | AA444093 |
| Sequence 6297 | AA446298 |
| Sequence 6298 | AA446910 |
| Sequence 6299 | AA447608 |
| Sequence 6300 | AA447948 |
| Sequence 6301 | AA448795 |
| Sequence 6302 | AA448882 |
| Sequence 6303 | AA449520 |
| Sequence 6304 | AA452012 |
| Sequence 6305 | AA452027 |
| Sequence 6306 | AA452352 |
| Sequence 6307 | AA453352 |
| Sequence 6308 | AA453651 |
| Sequence 6309 | AA453719 |
| Sequence 6310 | AA453784 |
| Sequence 6311 | AA454187 |
| Sequence 6312 | AA454962 |
| Sequence 6313 | AA455668 |
| Sequence 6314 | AA455894 |
| Sequence 6315 | AA456390 |
| Sequence 6316 | AA458761 |
| Sequence 6317 | AA458770 |
| Sequence 6318 | AA459167 |
| Sequence 6319 | AA459210 |
| Sequence 6320 | AA461128 |
| Sequence 6321 | AA461467 |
| Sequence 6322 | AA461605 |
| Sequence 6323 | AA464133 |
| Sequence 6324 | AA464141 |
| Sequence 6325 | AA467750 |
| Sequence 6326 | AA468167 |
| Sequence 6327 | AA468404 |
| Sequence 6328 | AA468624 |
| Sequence 6329 | AA468839 |
| Sequence 6330 | AA469129 |
| Sequence 6331 | AA469303 |
| Sequence 6332 | AA469345 |
| Sequence 6333 | AA469406 |
| Sequence 6334 | AA476524 |
| Sequence 6335 | AA476758 |
| Sequence 6336 | AA479167 |
| Sequence 6337 | AA479280 |
| Sequence 6338 | AA479287 |
| Sequence 6339 | AA479646 |
| Sequence 6340 | AA481078 |
| Sequence 6341 | AA481316 |
| Sequence 6342 | AA482539 |
| Sequence 6343 | AA482724 |
| Sequence 6344 | AA483214 |
| Sequence 6345 | AA485928 |
| Sequence 6346 | AA487448 |
| Sequence 6347 | AA487747 |
| Sequence 6348 | AA488633 |
| Sequence 6349 | AA492035 |
| Sequence 6350 | AA492280 |
| Sequence 6351 | AA493222 |
| Sequence 6352 | AA493331 |
| Sequence 6353 | AA493448 |
| Sequence 6354 | AA493512 |
| Sequence 6355 | AA493956 |
| Sequence 6356 | AA494214 |
| Sequence 6357 | AA494493 |
| Sequence 6358 | AA495759 |
| Sequence 6359 | AA496957 |
| Sequence 6360 | AA501363 |
| Sequence 6361 | AA501968 |
| Sequence 6362 | AA502901 |
| Sequence 6363 | AA502979 |
| Sequence 6364 | AA503020 |
| Sequence 6365 | AA504334 |
| Sequence 6366 | AA504953 |
| Sequence 6367 | AA505523 |
| Sequence 6368 | AA505953 |
| Sequence 6369 | AA507442 |
| Sequence 6370 | AA507777 |
| Sequence 6371 | AA508889 |
| Sequence 6372 | AA512974 |
| Sequence 6373 | AA514938 |
| Sequence 6374 | AA515132 |
| Sequence 6375 | AA515143 |
| Sequence 6376 | AA515564 |
| Sequence 6377 | AA516531 |
| Sequence 6378 | AA522936 |
| Sequence 6379 | AA523252 |
| Sequence 6380 | AA523464 |
| Sequence 6381 | AA523498 |
| Sequence 6382 | AA523652 |
| Sequence 6383 | AA523902 |
| Sequence 6384 | AA524485 |
| Sequence 6385 | AA524778 |
| Sequence 6386 | AA525419 |
| Sequence 6387 | AA526515 |
| Sequence 6388 | AA526591 |
| Sequence 6389 | AA526886 |
| Sequence 6390 | AA527187 |
| Sequence 6391 | AA527272 |
| Sequence 6392 | AA527730 |
| Sequence 6393 | AA527737 |
| Sequence 6394 | AA527805 |
| Sequence 6395 | AA528023 |
| Sequence 6396 | AA528104 |
| Sequence 6397 | AA528123 |
| Sequence 6398 | AA528202 |
| Sequence 6399 | AA531221 |
| Sequence 6400 | AA531312 |
| Sequence 6401 | AA531509 |
| Sequence 6402 | AA531563 |
| Sequence 6403 | AA531602 |
| Sequence 6404 | AA533057 |
| Sequence 6405 | AA533648 |
| Sequence 6406 | AA534235 |
| Sequence 6407 | AA534281 |
| Sequence 6408 | AA534435 |
| Sequence 6409 | AA535377 |
| Sequence 6410 | AA536036 |
| Sequence 6411 | AA542981 |
| Sequence 6412 | AA548472 |
| Sequence 6413 | AA548722 |
| Sequence 6414 | AA551464 |
| Sequence 6415 | AA552715 |
| Sequence 6416 | AA554661 |
| Sequence 6417 | AA554735 |
| Sequence 6418 | AA557888 |
| Sequence 6419 | AA559906 |
| Sequence 6420 | AA564484 |
| Sequence 6421 | AA564505 |
| Sequence 6422 | AA573742 |
| Sequence 6423 | AA573762 |
| Sequence 6424 | AA575882 |
| Sequence 6425 | AA576425 |
| Sequence 6426 | AA577553 |
| Sequence 6427 | AA578209 |
| Sequence 6428 | AA578511 |
| Sequence 6429 | AA578773 |
| Sequence 6430 | AA578881 |
| Sequence 6431 | AA578976 |
| Sequence 6432 | AA579384 |
| Sequence 6433 | AA579486 |
| Sequence 6434 | AA582591 |
| Sequence 6435 | AA583567 |
| Sequence 6436 | AA583574 |
| Sequence 6437 | AA587140 |
| Sequence 6438 | AA587226 |
| Sequence 6439 | AA587630 |
| Sequence 6440 | AA592908 |
| Sequence 6441 | AA594681 |
| Sequence 6442 | AA594682 |
| Sequence 6443 | AA595471 |
| Sequence 6444 | AA602794 |
| Sequence 6445 | AA602957 |
| Sequence 6446 | AA603041 |
| Sequence 6447 | AA603135 |
| Sequence 6448 | AA604474 |
| Sequence 6449 | AA604497 |
| Sequence 6450 | AA605077 |
| Sequence 6451 | AA609094 |
| Sequence 6452 | AA609436 |
| Sequence 6453 | AA610476 |
| Sequence 6454 | AA613916 |
| Sequence 6455 | AA613933 |
| Sequence 6456 | AA614105 |
| Sequence 6457 | AA614525 |
| Sequence 6458 | AA618484 |
| Sequence 6459 | AA620750 |
| Sequence 6460 | AA620995 |
| Sequence 6461 | AA622344 |
| Sequence 6462 | AA625222 |
| Sequence 6463 | AA625682 |
| Sequence 6464 | AA626439 |
| Sequence 6465 | AA628526 |
| Sequence 6466 | AA629534 |
| Sequence 6467 | AA629608 |
| Sequence 6468 | AA631868 |
| Sequence 6469 | AA631903 |
| Sequence 6470 | AA633794 |
| Sequence 6471 | AA634039 |
| Sequence 6472 | AA634216 |
| Sequence 6473 | AA634379 |
| Sequence 6474 | AA638989 |
| Sequence 6475 | AA639317 |
| Sequence 6476 | AA640105 |
| Sequence 6477 | AA640141 |
| Sequence 6478 | AA641623 |
| Sequence 6479 | AA642471 |
| Sequence 6480 | AA648508 |
| Sequence 6481 | AA649193 |
| Sequence 6482 | AA652846 |
| Sequence 6483 | AA653353 |
| Sequence 6484 | AA654557 |
| Sequence 6485 | AA654675 |
| Sequence 6486 | AA654875 |
| Sequence 6487 | AA659719 |
| Sequence 6488 | AA662658 |
| Sequence 6489 | AA662702 |
| Sequence 6490 | AA662822 |
| Sequence 6491 | AA664480 |
| Sequence 6492 | AA668224 |
| Sequence 6493 | AA668692 |
| Sequence 6494 | AA668951 |
| Sequence 6495 | AA670280 |
| Sequence 6496 | AA675891 |
| Sequence 6497 | AA677282 |
| Sequence 6498 | AA680052 |
| Sequence 6499 | AA682910 |
| Sequence 6500 | AA699999 |
| Sequence 6501 | AA702398 |
| Sequence 6502 | AA702888 |
| Sequence 6503 | AA705447 |
| Sequence 6504 | AA705736 |
| Sequence 6505 | AA705743 |
| Sequence 6506 | AA705813 |
| Sequence 6507 | AA706241 |
| Sequence 6508 | AA707490 |
| Sequence 6509 | AA708143 |
| Sequence 6510 | AA708195 |
| Sequence 6511 | AA713687 |
| Sequence 6512 | AA714934 |
| Sequence 6513 | AA716751 |
| Sequence 6514 | AA723130 |
| Sequence 6515 | AA723679 |
| Sequence 6516 | AA724619 |
| Sequence 6517 | AA724868 |
| Sequence 6518 | AA729119 |
| Sequence 6519 | AA732326 |
| Sequence 6520 | AA740261 |
| Sequence 6521 | AA741056 |
| Sequence 6522 | AA742231 |
| Sequence 6523 | AA744367 |
| Sequence 6524 | AA747819 |
| Sequence 6525 | AA764981 |
| Sequence 6526 | AA767323 |
| Sequence 6527 | AA772603 |
| Sequence 6528 | AA773009 |
| Sequence 6529 | AA773362 |
| Sequence 6530 | AA773374 |
| Sequence 6531 | AA775914 |
| Sequence 6532 | AA778038 |
| Sequence 6533 | AA778109 |
| Sequence 6534 | AA778596 |
| Sequence 6535 | AA808003 |
| Sequence 6536 | AA810159 |
| Sequence 6537 | AA812193 |
| Sequence 6538 | AA813266 |
| Sequence 6539 | AA813941 |
| Sequence 6540 | AA814496 |
| Sequence 6541 | AA826649 |
| Sequence 6542 | AA827331 |
| Sequence 6543 | AA831795 |
| Sequence 6544 | AA831876 |
| Sequence 6545 | AA835377 |
| Sequence 6546 | AA836233 |
| Sequence 6547 | AA836488 |
| Sequence 6548 | AA844979 |
| Sequence 6549 | AA845409 |
| Sequence 6550 | AA845442 |
| Sequence 6551 | AA846152 |
| Sequence 6552 | AA846576 |
| Sequence 6553 | AA854071 |
| Sequence 6554 | AA855093 |
| Sequence 6555 | AA862250 |
| Sequence 6556 | AA876668 |
| Sequence 6557 | AA884922 |
| Sequence 6558 | AA884972 |
| Sequence 6559 | AA884992 |
| Sequence 6560 | AA886748 |
| Sequence 6561 | AA886957 |
| Sequence 6562 | AA886998 |
| Sequence 6563 | AA887054 |
| Sequence 6564 | AA889969 |
| Sequence 6565 | AA890326 |
| Sequence 6566 | AA890421 |
| Sequence 6567 | AA896959 |
| Sequence 6568 | AA902127 |
| Sequence 6569 | AA907003 |
| Sequence 6570 | AA909768 |
| Sequence 6571 | AA916753 |
| Sequence 6572 | AA921906 |
| Sequence 6573 | AA923172 |
| Sequence 6574 | AA926926 |
| Sequence 6575 | AA927818 |
| Sequence 6576 | AA934755 |
| Sequence 6577 | AA934851 |
| Sequence 6578 | AA935181 |
| Sequence 6579 | AA935526 |
| Sequence 6580 | AA935560 |
| Sequence 6581 | AA935845 |
| Sequence 6582 | AA937773 |
| Sequence 6583 | AA938717 |
| Sequence 6584 | AA953892 |
| Sequence 6585 | AA961085 |
| Sequence 6586 | AA968756 |
| Sequence 6587 | AA969214 |
| Sequence 6588 | AA972883 |
| Sequence 6589 | AA974621 |
| Sequence 6590 | AA974908 |
| Sequence 6591 | AA975275 |
| Sequence 6592 | AA977871 |
| Sequence 6593 | AA984586 |
| Sequence 6594 | AA988125 |
| Sequence 6595 | AA989049 |
| Sequence 6596 | AA992217 |
| Sequence 6597 | AA992606 |
| Sequence 6598 | AI001862 |
| Sequence 6599 | AI003893 |
| Sequence 6600 | AI015053 |
| Sequence 6601 | AI016162 |
| Sequence 6602 | AI018773 |
| Sequence 6603 | AI022058 |
| Sequence 6604 | AI028507 |
| Sequence 6605 | AI028516 |
| Sequence 6606 | AI033912 |
| Sequence 6607 | AI038038 |
| Sequence 6608 | AI038986 |
| Sequence 6609 | AI039271 |
| Sequence 6610 | AI039636 |
| Sequence 6611 | AI040598 |
| Sequence 6612 | AI041497 |
| Sequence 6613 | AI042152 |
| Sequence 6614 | AI050872 |
| Sequence 6615 | AI051210 |
| Sequence 6616 | AI052124 |
| Sequence 6617 | AI052126 |
| Sequence 6618 | AI052724 |
| Sequence 6619 | AI052738 |
| Sequence 6620 | AI053597 |
| Sequence 6621 | AI054023 |
| Sequence 6622 | AI057281 |
| Sequence 6623 | AI057555 |
| Sequence 6624 | AI064691 |
| Sequence 6625 | AI074143 |
| Sequence 6626 | AI074389 |
| Sequence 6627 | AI075893 |
| Sequence 6628 | AI077500 |
| Sequence 6629 | AI079882 |
| Sequence 6630 | AI082272 |
| Sequence 6631 | AI082307 |
| Sequence 6632 | AI082674 |
| Sequence 6633 | AI084100 |
| Sequence 6634 | AI084101 |
| Sequence 6635 | AI088178 |
| Sequence 6636 | AI090786 |
| Sequence 6637 | AI093233 |
| Sequence 6638 | AI094394 |
| Sequence 6639 | AI095250 |
| Sequence 6640 | AI096410 |
| Sequence 6641 | AI123199 |
| Sequence 6642 | AI123672 |
| Sequence 6643 | AI123822 |
| Sequence 6644 | AI125642 |
| Sequence 6645 | AI126257 |
| Sequence 6646 | AI127268 |
| Sequence 6647 | AI127556 |
| Sequence 6648 | AI129800 |
| Sequence 6649 | AI131216 |
| Sequence 6650 | AI133208 |
| Sequence 6651 | AI133690 |
| Sequence 6652 | AI139036 |
| Sequence 6653 | AI142021 |
| Sequence 6654 | AI142095 |
| Sequence 6655 | AI142558 |
| Sequence 6656 | AI146476 |
| Sequence 6657 | AI148115 |
| Sequence 6658 | AI148251 |
| Sequence 6659 | AI148561 |
| Sequence 6660 | AI148676 |
| Sequence 6661 | AI167651 |
| Sequence 6662 | AI174853 |
| Sequence 6663 | AI174899 |
| Sequence 6664 | AI184748 |
| Sequence 6665 | AI189386 |
| Sequence 6666 | AI189897 |
| Sequence 6667 | AI190785 |
| Sequence 6668 | AI198311 |
| Sequence 6669 | AI198853 |
| Sequence 6670 | AI198930 |
| Sequence 6671 | AI198986 |
| Sequence 6672 | AI199094 |
| Sequence 6673 | AI199681 |
| Sequence 6674 | AI200798 |
| Sequence 6675 | AI200830 |
| Sequence 6676 | AI201564 |
| Sequence 6677 | AI203377 |
| Sequence 6678 | AI204096 |
| Sequence 6679 | AI206483 |
| Sequence 6680 | AI206818 |
| Sequence 6681 | AI207546 |
| Sequence 6682 | AI207618 |
| Sequence 6683 | AI207650 |
| Sequence 6684 | AI214272 |
| Sequence 6685 | AI216968 |
| Sequence 6686 | AI216984 |
| Sequence 6687 | AI217003 |
| Sequence 6688 | AI217172 |
| Sequence 6689 | AI219570 |
| Sequence 6690 | AI221894 |
| Sequence 6691 | AI222329 |
| Sequence 6692 | AI222789 |
| Sequence 6693 | AI222806 |
| Sequence 6694 | AI223061 |
| Sequence 6695 | AI244217 |
| Sequence 6696 | AI253288 |
| Sequence 6697 | AI253319 |
| Sequence 6698 | AI253338 |
| Sequence 6699 | AI253348 |
| Sequence 6700 | AI261883 |
| Sequence 6701 | AI267162 |
| Sequence 6702 | AI267353 |
| Sequence 6703 | AI267454 |
| Sequence 6704 | AI267519 |
| Sequence 6705 | AI267652 |
| Sequence 6706 | AI267656 |
| Sequence 6707 | AI267664 |
| Sequence 6708 | AI267849 |
| Sequence 6709 | AI269060 |
| Sequence 6710 | AI272941 |
| Sequence 6711 | AI273945 |
| Sequence 6712 | AI275792 |
| Sequence 6713 | AI282318 |
| Sequence 6714 | AI283096 |
| Sequence 6715 | AI285408 |
| Sequence 6716 | AI287603 |
| Sequence 6717 | AI289111 |
| Sequence 6718 | AI290826 |
| Sequence 6719 | AI291296 |
| Sequence 6720 | AI298972 |
| Sequence 6721 | AI300489 |
| Sequence 6722 | AI302102 |
| Sequence 6723 | AI305838 |
| Sequence 6724 | AI306457 |
| Sequence 6725 | AI307419 |
| Sequence 6726 | AI310138 |
| Sequence 6727 | AI312552 |
| Sequence 6728 | AI313387 |
| Sequence 6729 | AI332712 |
| Sequence 6730 | AI335829 |
| Sequence 6731 | AI338379 |
| Sequence 6732 | AI340611 |
| Sequence 6733 | AI341551 |
| Sequence 6734 | AI342409 |
| Sequence 6735 | AI342468 |
| Sequence 6736 | AI342669 |
| Sequence 6737 | AI343112 |
| Sequence 6738 | AI343692 |
| Sequence 6739 | AI346657 |
| Sequence 6740 | AI348579 |
| Sequence 6741 | AI352141 |
| Sequence 6742 | AI357472 |
| Sequence 6743 | AI358685 |
| Sequence 6744 | AI360710 |
| Sequence 6745 | AI361905 |
| Sequence 6746 | AI361969 |
| Sequence 6747 | AI365612 |
| Sequence 6748 | AI376840 |
| Sequence 6749 | AI380706 |
| Sequence 6750 | AI380932 |
| Sequence 6751 | AI382020 |
| Sequence 6752 | AI382065 |
| Sequence 6753 | AI382189 |
| Sequence 6754 | AI383945 |
| Sequence 6755 | AI400752 |
| Sequence 6756 | AI401771 |
| Sequence 6757 | AI420898 |
| Sequence 6758 | AI423180 |
| Sequence 6759 | AI432306 |
| Sequence 6760 | AI432462 |
| Sequence 6761 | AI433976 |
| Sequence 6762 | AI435153 |
| Sequence 6763 | AI435945 |
| Sequence 6764 | AI440088 |
| Sequence 6765 | AI452485 |
| Sequence 6766 | AI457157 |
| Sequence 6767 | AI467861 |
| Sequence 6768 | AI468347 |
| Sequence 6769 | AI474235 |
| Sequence 6770 | AI475476 |
| Sequence 6771 | AI523940 |
| Sequence 6772 | AI525552 |
| Sequence 6773 | AI525702 |
| Sequence 6774 | AI525843 |
| Sequence 6775 | AI536991 |
| Sequence 6776 | AI553845 |
| Sequence 6777 | AI557112 |
| Sequence 6778 | AI557182 |
| Sequence 6779 | AI557225 |
| Sequence 6780 | AI557237 |
| Sequence 6781 | AI557452 |
| Sequence 6782 | AI557599 |
| Sequence 6783 | AI557639 |
| Sequence 6784 | AI567102 |
| Sequence 6785 | AI569556 |
| Sequence 6786 | AI569819 |
| Sequence 6787 | AI569955 |
| Sequence 6788 | AI570221 |
| Sequence 6789 | AI571418 |
| Sequence 6790 | AI580176 |
| Sequence 6791 | AI590195 |
| Sequence 6792 | AI590351 |
| Sequence 6793 | AI598180 |
| Sequence 6794 | AI610920 |
| Sequence 6795 | AI620111 |
| Sequence 6796 | AI620796 |
| Sequence 6797 | AI623924 |
| Sequence 6798 | AI625201 |
| Sequence 6799 | AI625864 |
| Sequence 6800 | AI631703 |
| Sequence 6801 | AI631745 |
| Sequence 6802 | AI634228 |
| Sequence 6803 | AI635275 |
| Sequence 6804 | AI635681 |
| Sequence 6805 | AI636899 |
| Sequence 6806 | AI648684 |
| Sequence 6807 | AI660606 |
| Sequence 6808 | AI669704 |
| Sequence 6809 | AI671180 |
| Sequence 6810 | AI672201 |
| Sequence 6811 | AI672868 |
| Sequence 6812 | AI681405 |
| Sequence 6813 | AI683298 |
| Sequence 6814 | AI684170 |
| Sequence 6815 | AI686139 |
| Sequence 6816 | AI689722 |
| Sequence 6817 | AI693697 |
| Sequence 6818 | AI694087 |
| Sequence 6819 | AI694199 |
| Sequence 6820 | AI694767 |
| Sequence 6821 | AI697470 |
| Sequence 6822 | AI701140 |
| Sequence 6823 | AI703256 |
| Sequence 6824 | AI708172 |
| Sequence 6825 | AI709186 |
| Sequence 6826 | AI718780 |
| Sequence 6827 | AI718825 |
| Sequence 6828 | AI733116 |
| Sequence 6829 | AI734854 |
| Sequence 6830 | AI735314 |
| Sequence 6831 | AI735683 |
| Sequence 6832 | AI739102 |
| Sequence 6833 | AI741354 |
| Sequence 6834 | AI741843 |
| Sequence 6835 | AI742468 |
| Sequence 6836 | AI742912 |
| Sequence 6837 | AI743852 |
| Sequence 6838 | AI744509 |
| Sequence 6839 | AI749547 |
| Sequence 6840 | AI751364 |
| Sequence 6841 | AI751565 |
| Sequence 6842 | AI753664 |
| Sequence 6843 | AI753784 |
| Sequence 6844 | AI754741 |
| Sequence 6845 | AI755085 |
| Sequence 6846 | AI760827 |
| Sequence 6847 | AI761110 |
| Sequence 6848 | AI766123 |
| Sequence 6849 | AI766514 |
| Sequence 6850 | AI768465 |
| Sequence 6851 | AI769392 |
| Sequence 6852 | AI770054 |
| Sequence 6853 | AI791548 |
| Sequence 6854 | AI793062 |
| Sequence 6855 | AI796560 |
| Sequence 6856 | AI799974 |
| Sequence 6857 | AI800048 |
| Sequence 6858 | AI802154 |
| Sequence 6859 | AI805364 |
| Sequence 6860 | AI813758 |
| Sequence 6861 | AI815380 |
| Sequence 6862 | AI819014 |
| Sequence 6863 | AI827550 |
| Sequence 6864 | AI828670 |
| Sequence 6865 | AI831047 |
| Sequence 6866 | AI861878 |
| Sequence 6867 | AI879179 |
| Sequence 6868 | AI879995 |
| Sequence 6869 | AI887632 |
| Sequence 6870 | AI888438 |
| Sequence 6871 | AI890240 |
| Sequence 6872 | AI910552 |
| Sequence 6873 | AI912414 |
| Sequence 6874 | AI914128 |
| Sequence 6875 | AI914133 |
| Sequence 6876 | AI921434 |
| Sequence 6877 | AI922855 |
| Sequence 6878 | AI923011 |
| Sequence 6879 | AI923978 |
| Sequence 6880 | AI929509 |
| Sequence 6881 | AI937296 |
| Sequence 6882 | AI940170 |
| Sequence 6883 | AI948503 |
| Sequence 6884 | AI954458 |
| Sequence 6885 | AI961840 |
| Sequence 6886 | AI963623 |
| Sequence 6887 | AI970594 |
| Sequence 6888 | AI972279 |
| Sequence 6889 | AI973109 |
| Sequence 6890 | AI983220 |
| Sequence 6891 | AL035731 |
| Sequence 6892 | AL035985 |
| Sequence 6893 | AL036123 |
| Sequence 6894 | AL038169 |
| Sequence 6895 | AL036483 |
| Sequence 6896 | AL036801 |
| Sequence 6897 | AL036988 |
| Sequence 6898 | AL037267 |
| Sequence 6899 | AL037471 |
| Sequence 6900 | AL037800 |
| Sequence 6901 | AL039253 |
| Sequence 6902 | AL040873 |
| Sequence 6903 | AL041050 |
| Sequence 6904 | AL041780 |
| Sequence 6905 | AL042316 |
| Sequence 6906 | AL044447 |
| Sequence 6907 | AL044649 |
| Sequence 6908 | AL045213 |
| Sequence 6909 | AL048699 |
| Sequence 6910 | AL049012 |
| Sequence 6911 | AL110412 |
| Sequence 6912 | AL118621 |
| Sequence 6913 | AL119462 |
| Sequence 6914 | AL120798 |
| Sequence 6915 | AL120840 |
| Sequence 6916 | AL121547 |
| Sequence 6917 | AW003247 |
| Sequence 6918 | AW003692 |
| Sequence 6919 | AW005103 |
| Sequence 6920 | AW008213 |
| Sequence 6921 | AW015683 |
| Sequence 6922 | AW020479 |
| Sequence 6923 | AW022300 |
| Sequence 6924 | AW022756 |
| Sequence 6925 | AW023489 |
| Sequence 6926 | AW051386 |
| Sequence 6927 | AW052140 |
| Sequence 6928 | AW062933 |
| Sequence 6929 | AW067913 |
| Sequence 6930 | AW071063 |
| Sequence 6931 | AW071903 |
| Sequence 6932 | AW080160 |
| Sequence 6933 | AW083804 |
| Sequence 6934 | AW090544 |
| Sequence 6935 | AW104561 |
| Sequence 6936 | AW135430 |
| Sequence 6937 | AW138413 |
| Sequence 6938 | AW151295 |
| Sequence 6939 | AW157047 |
| Sequence 6940 | AW157303 |
| Sequence 6941 | AW160731 |
| Sequence 6942 | AW161491 |
| Sequence 6943 | AW168960 |
| Sequence 6944 | AW169876 |
| Sequence 6945 | AW170485 |
| Sequence 6946 | AW179006 |
| Sequence 6947 | AW183759 |
| Sequence 6948 | C06419 |
| Sequence 6949 | D55192 |
| Sequence 6950 | D58694 |
| Sequence 6951 | F05508 |
| Sequence 6952 | F06621 |
| Sequence 6953 | F06739 |
| Sequence 6954 | F12493 |
| Sequence 6955 | F22763 |
| Sequence 6956 | H00112 |
| Sequence 6957 | H03785 |
| Sequence 6958 | H12527 |
| Sequence 6959 | H13339 |
| Sequence 6960 | H16455 |
| Sequence 6961 | H20086 |
| Sequence 6962 | H20888 |
| Sequence 6963 | H22853 |
| Sequence 6964 | H23837 |
| Sequence 6965 | H39906 |
| Sequence 6966 | H39960 |
| Sequence 6967 | H45550 |
| Sequence 6968 | H50566 |
| Sequence 6969 | H57382 |
| Sequence 6970 | H59731 |
| Sequence 6971 | H59791 |
| Sequence 6972 | H70670 |
| Sequence 6973 | H75695 |
| Sequence 6974 | H78954 |
| Sequence 6975 | H81659 |
| Sequence 6976 | H83574 |
| Sequence 6977 | H84729 |
| Sequence 6978 | H86010 |
| Sequence 6979 | L49115 |
| Sequence 6980 | M85423 |
| Sequence 6981 | N25145 |
| Sequence 6982 | N26359 |
| Sequence 6983 | N26421 |
| Sequence 6984 | N28384 |
| Sequence 6985 | N30486 |
| Sequence 6986 | N33567 |
| Sequence 6987 | N40852 |
| Sequence 6988 | N41386 |
| Sequence 6989 | N41827 |
| Sequence 6990 | N42196 |
| Sequence 6991 | N44001 |
| Sequence 6992 | N45940 |
| Sequence 6993 | N52271 |
| Sequence 6994 | N52554 |
| Sequence 6995 | N63991 |
| Sequence 6996 | N72922 |
| Sequence 6997 | N79747 |
| Sequence 6998 | N90917 |
| Sequence 6999 | N98328 |
| Sequence 7000 | N98569 |
| Sequence 7001 | N98593 |
| Sequence 7002 | R11581 |
| Sequence 7003 | R12113 |
| Sequence 7004 | R17092 |
| Sequence 7005 | R20331 |
| Sequence 7006 | R22136 |
| Sequence 7007 | R32613 |
| Sequence 7008 | R35797 |
| Sequence 7009 | R49734 |
| Sequence 7010 | R50467 |
| Sequence 7011 | R59147 |
| Sequence 7012 | R61601 |
| Sequence 7013 | R69943 |
| Sequence 7014 | R70570 |
| Sequence 7015 | R70639 |
| Sequence 7016 | R70995 |
| Sequence 7017 | R71431 |
| Sequence 7018 | R74597 |
| Sequence 7019 | R80852 |
| Sequence 7020 | R89611 |
| Sequence 7021 | R93759 |
| Sequence 7022 | R98423 |
| Sequence 7023 | T05006 |
| Sequence 7024 | T05635 |
| Sequence 7025 | T10307 |
| Sequence 7026 | T27258 |
| Sequence 7027 | T28083 |
| Sequence 7028 | T31772 |
| Sequence 7029 | T61421 |
| Sequence 7030 | T65998 |
| Sequence 7031 | T72376 |
| Sequence 7032 | T78948 |
| Sequence 7033 | T83709 |
| Sequence 7034 | T84943 |
| Sequence 7035 | U69559 |
| Sequence 7036 | W00556 |
| Sequence 7037 | W03170 |
| Sequence 7038 | W03471 |
| Sequence 7039 | W05472 |
| Sequence 7040 | W07109 |
| Sequence 7041 | W19899 |
| Sequence 7042 | W25222 |
| Sequence 7043 | W25970 |
| Sequence 7044 | W27440 |
| Sequence 7045 | W28596 |
| Sequence 7046 | W28729 |
| Sequence 7047 | W28994 |
| Sequence 7048 | W39249 |
| Sequence 7049 | W40294 |
| Sequence 7050 | W45179 |
| Sequence 7051 | W45265 |
| Sequence 7052 | W51959 |
| Sequence 7053 | W56523 |
| Sequence 7054 | W56718 |
| Sequence 7055 | W63793 |
| Sequence 7056 | W65292 |
| Sequence 7057 | W69806 |
| Sequence 7058 | W76134 |
| Sequence 7059 | W78775 |
| Sequence 7060 | W87891 |
| Sequence 7061 | W93655 |
| Sequence 7062 | W99251 |
| Sequence 7063 | Z19471 |
| Sequence 7064 | Z30311 |
| Sequence 7065 | A06846 |
| Sequence 7066 | AB001106 |
| Sequence 7067 | AB002357 |
| Sequence 7068 | AB002365 |
| Sequence 7069 | AB002386 |
| Sequence 7070 | AB002387 |
| Sequence 7071 | AB002451 |
| Sequence 7072 | AB006077 |
| Sequence 7073 | AB007892 |
| Sequence 7074 | AB007896 |
| Sequence 7075 | AB007931 |
| Sequence 7076 | AB007956 |
| Sequence 7077 | AB011004 |
| Sequence 7078 | AB011079 |
| Sequence 7079 | AB011103 |
| Sequence 7080 | AB011120 |
| Sequence 7081 | AB014486 |
| Sequence 7082 | AB014519 |
| Sequence 7083 | AB014543 |
| Sequence 7084 | AB014548 |
| Sequence 7085 | AB014601 |
| Sequence 7086 | AB015639 |
| Sequence 7087 | AB016068 |
| Sequence 7088 | AB017004 |
| Sequence 7089 | AB018331 |
| Sequence 7090 | AB018346 |
| Sequence 7091 | AB019524 |
| Sequence 7092 | AB019563 |
| Sequence 7093 | AB019566 |
| Sequence 7094 | AB019568 |
| Sequence 7095 | AB020637 |
| Sequence 7096 | AB020661 |
| Sequence 7097 | AB020686 |
| Sequence 7098 | AB020689 |
| Sequence 7099 | AB020692 |
| Sequence 7100 | AB020705 |
| Sequence 7101 | AB021288 |
| Sequence 7102 | AB021663 |
| Sequence 7103 | AB023145 |
| Sequence 7104 | AB023163 |
| Sequence 7105 | AB023173 |
| Sequence 7106 | AB023205 |
| Sequence 7107 | AB023210 |
| Sequence 7108 | AB023230 |
| Sequence 7109 | AB026190 |
| Sequence 7110 | AB027013 |
| Sequence 7111 | AB028624 |
| Sequence 7112 | AB028981 |
| Sequence 7113 | AB029000 |
| Sequence 7114 | AB029004 |
| Sequence 7115 | AB032983 |
| Sequence 7116 | AB032991 |
| Sequence 7117 | AB033007 |
| Sequence 7118 | AB033071 |
| Sequence 7119 | AB033079 |
| Sequence 7120 | AB033091 |
| Sequence 7121 | AB033899 |
| Sequence 7122 | AD001528 |
| Sequence 7123 | AF000381 |
| Sequence 7124 | AF000982 |
| Sequence 7125 | AF000984 |
| Sequence 7126 | AF007142 |
| Sequence 7127 | AF007791 |
| Sequence 7128 | AF010233 |
| Sequence 7129 | AF013717 |
| Sequence 7130 | AF013759 |
| Sequence 7131 | AF013988 |
| Sequence 7132 | AF016270 |
| Sequence 7133 | AF017782 |
| Sequence 7134 | AF020038 |
| Sequence 7135 | AF020202 |
| Sequence 7136 | AF020351 |
| Sequence 7137 | AF020797 |
| Sequence 7138 | AF021819 |
| Sequence 7139 | AF022215 |
| Sequence 7140 | AF026939 |
| Sequence 7141 | AF026977 |
| Sequence 7142 | AF027824 |
| Sequence 7143 | AF029689 |
| Sequence 7144 | AF034091 |
| Sequence 7145 | AF035286 |
| Sequence 7146 | AF035293 |
| Sequence 7147 | AF035309 |
| Sequence 7148 | AF038451 |
| Sequence 7149 | AF038452 |
| Sequence 7150 | AF038957 |
| Sequence 7151 | AF039692 |
| Sequence 7152 | AF039918 |
| Sequence 7153 | AF042386 |
| Sequence 7154 | AF044321 |
| Sequence 7155 | AF044670 |
| Sequence 7156 | AF044671 |
| Sequence 7157 | AF044956 |
| Sequence 7158 | AF045167 |
| Sequence 7159 | AF045229 |
| Sequence 7160 | AF047020 |
| Sequence 7161 | AF049910 |
| Sequence 7162 | AF051323 |
| Sequence 7163 | AF052124 |
| Sequence 7164 | AF052129 |
| Sequence 7165 | AF054186 |
| Sequence 7166 | AF054187 |
| Sequence 7167 | AF054838 |
| Sequence 7168 | AF054990 |
| Sequence 7169 | AF056322 |
| Sequence 7170 | AF059524 |
| Sequence 7171 | AF060219 |
| Sequence 7172 | AF061258 |
| Sequence 7173 | AF062323 |
| Sequence 7174 | AF065388 |
| Sequence 7175 | AF067171 |
| Sequence 7176 | AF068235 |
| Sequence 7177 | AF070523 |
| Sequence 7178 | AF070539 |
| Sequence 7179 | AF070555 |
| Sequence 7180 | AF070556 |
| Sequence 7181 | AF070561 |
| Sequence 7182 | AF070650 |
| Sequence 7183 | AF070655 |
| Sequence 7184 | AF071202 |
| Sequence 7185 | AF073475 |
| Sequence 7186 | AF075587 |
| Sequence 7187 | AF077030 |
| Sequence 7188 | AF077035 |
| Sequence 7189 | AF077042 |
| Sequence 7190 | AF077202 |
| Sequence 7191 | AF078848 |
| Sequence 7192 | AF078849 |
| Sequence 7193 | AF078854 |
| Sequence 7194 | AF078859 |
| Sequence 7195 | AF078862 |
| Sequence 7196 | AF082657 |
| Sequence 7197 | AF084457 |
| Sequence 7198 | AF084555 |
| Sequence 7199 | AF085355 |
| Sequence 7200 | AF085845 |
| Sequence 7201 | AF086002 |
| Sequence 7202 | AF086003 |
| Sequence 7203 | AF086172 |
| Sequence 7204 | AF086182 |
| Sequence 7205 | AF086183 |
| Sequence 7206 | AF086336 |
| Sequence 7207 | AF086401 |
| Sequence 7208 | AF086431 |
| Sequence 7209 | AF086517 |
| Sequence 7210 | AF086557 |
| Sequence 7211 | AF087999 |
| Sequence 7212 | AF089747 |
| Sequence 7213 | AF092092 |
| Sequence 7214 | AF097514 |
| Sequence 7215 | AF100741 |
| Sequence 7216 | AF100749 |
| Sequence 7217 | AF100756 |
| Sequence 7218 | AF100757 |
| Sequence 7219 | AF102265 |
| Sequence 7220 | AF103907 |
| Sequence 7221 | AF109219 |
| Sequence 7222 | AF113131 |
| Sequence 7223 | AF113132 |
| Sequence 7224 | AF124438 |
| Sequence 7225 | AF126780 |
| Sequence 7226 | AF126782 |
| Sequence 7227 | AF129927 |
| Sequence 7228 | AF131758 |
| Sequence 7229 | AF131763 |
| Sequence 7230 | AF131814 |
| Sequence 7231 | AF131838 |
| Sequence 7232 | AF131856 |
| Sequence 7233 | AF132000 |
| Sequence 7234 | AF141968 |
| Sequence 7235 | AF147330 |
| Sequence 7236 | AF147331 |
| Sequence 7237 | AF147339 |
| Sequence 7238 | AF147395 |
| Sequence 7239 | AF151103 |
| Sequence 7240 | AF151109 |
| Sequence 7241 | AF151803 |
| Sequence 7242 | AF151818 |
| Sequence 7243 | AF151840 |
| Sequence 7244 | AF151867 |
| Sequence 7245 | AFIS1871 |
| Sequence 7246 | AF151878 |
| Sequence 7247 | AF153608 |
| Sequence 7248 | AF155095 |
| Sequence 7249 | AF155110 |
| Sequence 7250 | AF156965 |
| Sequence 7251 | AF159056 |
| Sequence 7252 | AF168956 |
| Sequence 7253 | AF169797 |
| Sequence 7254 | AF170583 |
| Sequence 7255 | AF174605 |
| Sequence 7256 | AF176574 |
| Sequence 7257 | AF179274 |
| Sequence 7258 | AF182289 |
| Sequence 7259 | AF182645 |
| Sequence 7260 | AF188745 |
| Sequence 7261 | AF188746 |
| Sequence 7262 | AF195120 |
| Sequence 7263 | AF201077 |
| Sequence 7264 | AJ002744 |
| Sequence 7265 | AJ004832 |
| Sequence 7266 | AJ005016 |
| Sequence 7267 | AJ010071 |
| Sequence 7268 | AJ011001 |
| Sequence 7269 | AJ223183 |
| Sequence 7270 | AJ249248 |
| Sequence 7271 | AL021683 |
| Sequence 7272 | AL035304 |
| Sequence 7273 | AL049381 |
| Sequence 7274 | AL049705 |
| Sequence 7275 | AL049932 |
| Sequence 7276 | AL049934 |
| Sequence 7277 | AL049957 |
| Sequence 7278 | AL049969 |
| Sequence 7279 | AL050159 |
| Sequence 7280 | AL050162 |
| Sequence 7281 | AL050187 |
| Sequence 7282 | AL050198 |
| Sequence 7283 | AL050265 |
| Sequence 7284 | AL050287 |
| Sequence 7285 | AL079298 |
| Sequence 7286 | AL080202 |
| Sequence 7287 | AL080209 |
| Sequence 7288 | AL080223 |
| Sequence 7289 | AL080234 |
| Sequence 7290 | AL096719 |
| Sequence 7291 | AL096857 |
| Sequence 7292 | AL110126 |
| Sequence 7293 | AL110183 |
| Sequence 7294 | AL110185 |
| Sequence 7295 | AL117423 |
| Sequence 7296 | AL117429 |
| Sequence 7297 | AL117446 |
| Sequence 7298 | AL117458 |
| Sequence 7299 | AL117621 |
| Sequence 7300 | AL117644 |
| Sequence 7301 | AL117666 |
| Sequence 7302 | AL122088 |
| Sequence 7303 | AL133074 |
| Sequence 7304 | AL133076 |
| Sequence 7305 | D00017 |
| Sequence 7306 | D12676 |
| Sequence 7307 | D13119 |
| Sequence 7308 | D13315 |
| Sequence 7309 | D13866 |
| Sequence 7310 | D14530 |
| Sequence 7311 | D14696 |
| Sequence 7312 | D14697 |
| Sequence 7313 | D14812 |
| Sequence 7314 | D16911 |
| Sequence 7315 | D17039 |
| Sequence 7316 | D17554 |
| Sequence 7317 | D21262 |
| Sequence 7318 | D21853 |
| Sequence 7319 | D25542 |
| Sequence 7320 | D26598 |
| Sequence 7321 | D28476 |
| Sequence 7322 | D29677 |
| Sequence 7323 | D29954 |
| Sequence 7324 | D30655 |
| Sequence 7325 | D31883 |
| Sequence 7326 | D37931 |
| Sequence 7327 | D38293 |
| Sequence 7328 | D38441 |
| Sequence 7329 | D38583 |
| Sequence 7330 | D42047 |
| Sequence 7331 | D43950 |
| Sequence 7332 | D50931 |
| Sequence 7333 | D59253 |
| Sequence 7334 | D78275 |
| Sequence 7335 | D79205 |
| Sequence 7336 | D79986 |
| Sequence 7337 | D80009 |
| Sequence 7338 | D83780 |
| Sequence 7339 | D84145 |
| Sequence 7340 | D85758 |
| Sequence 7341 | D86984 |
| Sequence 7342 | D87127 |
| Sequence 7343 | D87442 |
| Sequence 7344 | D87455 |
| Sequence 7345 | D87666 |
| Sequence 7346 | D87735 |
| Sequence 7347 | D87742 |
| Sequence 7348 | D88208 |
| Sequence 7349 | D89053 |
| Sequence 7350 | D90209 |
| Sequence 7351 | D90373 |
| Sequence 7352 | E01650 |
| Sequence 7353 | E02628 |
| Sequence 7354 | E02822 |
| Sequence 7355 | E06721 |
| Sequence 7356 | E07798 |
| Sequence 7357 | E08663 |
| Sequence 7358 | J02876 |
| Sequence 7359 | J02888 |
| Sequence 7360 | J03007 |
| Sequence 7361 | J03040 |
| Sequence 7362 | J03248 |
| Sequence 7363 | J03464 |
| Sequence 7364 | J03746 |
| Sequence 7365 | J03799 |
| Sequence 7366 | J04164 |
| Sequence 7367 | J04205 |
| Sequence 7368 | J04208 |
| Sequence 7369 | J04443 |
| Sequence 7370 | J04794 |
| Sequence 7371 | K01911 |
| Sequence 7372 | K03515 |
| Sequence 7373 | L00635 |
| Sequence 7374 | L01100 |
| Sequence 7375 | L04636 |
| Sequence 7376 | L05093 |
| Sequence 7377 | L05779 |
| Sequence 7378 | L08441 |
| Sequence 7379 | L09159 |
| Sequence 7380 | L10320 |
| Sequence 7381 | L10678 |
| Sequence 7382 | L16558 |
| Sequence 7383 | L19597 |
| Sequence 7384 | L19605 |
| Sequence 7385 | L20773 |
| Sequence 7386 | L20941 |
| Sequence 7387 | 126081 |
| Sequence 7388 | L33404 |
| Sequence 7389 | L34840 |
| Sequence 7390 | L38486 |
| Sequence 7391 | L38961 |
| Sequence 7392 | L47276 |
| Sequence 7393 | L49504 |
| Sequence 7394 | L77964 |
| Sequence 7395 | M10119 |
| Sequence 7396 | M10941 |
| Sequence 7397 | M11058 |
| Sequence 7398 | M11353 |
| Sequence 7399 | M12938 |
| Sequence 7400 | M14200 |
| Sequence 7401 | M14631 |
| Sequence 7402 | M15178 |
| Sequence 7403 | M16768 |
| Sequence 7404 | M16804 |
| Sequence 7405 | M16942 |
| Sequence 7406 | M17733 |
| Sequence 7407 | M17885 |
| Sequence 7408 | M21154 |
| Sequence 7409 | M21574 |
| Sequence 7410 | M21895 |
| Sequence 7411 | M24194 |
| Sequence 7412 | M24902 |
| Sequence 7413 | M25077 |
| Sequence 7414 | M25246 |
| Sequence 7415 | M26663 |
| Sequence 7416 | M27332 |
| Sequence 7417 | M27335 |
| Sequence 7418 | M28211 |
| Sequence 7419 | M29366 |
| Sequence 7420 | M31630 |
| Sequence 7421 | M32790 |
| Sequence 7422 | M33308 |
| Sequence 7423 | M34840 |
| Sequence 7424 | M35252 |
| Sequence 7425 | M36341 |
| Sequence 7426 | M36634 |
| Sequence 7427 | M37716 |
| Sequence 7428 | M55265 |
| Sequence 7429 | M55409 |
| Sequence 7430 | M58525 |
| Sequence 7431 | M61831 |
| Sequence 7432 | M63573 |
| Sequence 7433 | M65217 |
| Sequence 7434 | M69238 |
| Sequence 7435 | M74091 |
| Sequence 7436 | M74509 |
| Sequence 7437 | M74777 |
| Sequence 7438 | M76729 |
| Sequence 7439 | M77830 |
| Sequence 7440 | M80783 |
| Sequence 7441 | M81757 |
| Sequence 7442 | M83822 |
| Sequence 7443 | M90054 |
| Sequence 7444 | M93651 |
| Sequence 7445 | M95724 |
| Sequence 7446 | M97347 |
| Sequence 7447 | S66196 |
| Sequence 7448 | S66431 |
| Sequence 7449 | S67815 |
| Sequence 7450 | S69272 |
| Sequence 7451 | S70154 |
| Sequence 7452 | S70290 |
| Sequence 7453 | S73498 |
| Sequence 7454 | S73591 |
| Sequence 7455 | S78203 |
| Sequence 7456 | S82081 |
| Sequence 7457 | U03886 |
| Sequence 7458 | U05875 |
| Sequence 7459 | U08815 |
| Sequence 7460 | U09559 |
| Sequence 7461 | U09820 |
| Sequence 7462 | U13991 |
| Sequence 7463 | U14658 |
| Sequence 7464 | U14967 |
| Sequence 7465 | U14970 |
| Sequence 7466 | U14971 |
| Sequence 7467 | U15008 |
| Sequence 7468 | U18291 |
| Sequence 7469 | U18297 |
| Sequence 7470 | U20157 |
| Sequence 7471 | U21858 |
| Sequence 7472 | U24105 |
| Sequence 7473 | U25789 |
| Sequence 7474 | U30826 |
| Sequence 7475 | U31905 |
| Sequence 7476 | U32944 |
| Sequence 7477 | U33821 |
| Sequence 7478 | U34252 |
| Sequence 7479 | U36764 |
| Sequence 7480 | U37143 |
| Sequence 7481 | U37230 |
| Sequence 7482 | U37689 |
| Sequence 7483 | U38894 |
| Sequence 7484 | U39400 |
| Sequence 7485 | U40272 |
| Sequence 7486 | U40763 |
| Sequence 7487 | U47742 |
| Sequence 7488 | U54558 |
| Sequence 7489 | U58855 |
| Sequence 7490 | U59435 |
| Sequence 7491 | U61083 |
| Sequence 7492 | U65090 |
| Sequence 7493 | U69645 |
| Sequence 7494 | U70730 |
| Sequence 7495 | U79260 |
| Sequence 7496 | U79278 |
| Sequence 7497 | U80034 |
| Sequence 7498 | U80669 |
| Sequence 7499 | U83115 |
| Sequence 7500 | U85658 |
| Sequence 7501 | U89436 |
| Sequence 7502 | U90904 |
| Sequence 7503 | U96759 |
| Sequence 7504 | U96915 |
| Sequence 7505 | U97519 |
| Sequence 7506 | V00594 |
| Sequence 7507 | X01630 |
| Sequence 7508 | X01742 |
| Sequence 7509 | X02530 |
| Sequence 7510 | X02761 |
| Sequence 7511 | X04098 |
| Sequence 7512 | X04470 |
| Sequence 7513 | X05332 |
| Sequence 7514 | X06617 |
| Sequence 7515 | X07819 |
| Sequence 7516 | X12597 |
| Sequence 7517 | X12791 |
| Sequence 7518 | X15187 |
| Sequence 7519 | X15729 |
| Sequence 7520 | X15759 |
| Sequence 7521 | X52174 |
| Sequence 7522 | X53605 |
| Sequence 7523 | X55654 |
| Sequence 7524 | X56999 |
| Sequence 7525 | X57352 |
| Sequence 7526 | X57766 |
| Sequence 7527 | X57812 |
| Sequence 7528 | X59406 |
| Sequence 7529 | X59834 |
| Sequence 7530 | X59841 |
| Sequence 7531 | X63432 |
| Sequence 7532 | X64330 |
| Sequence 7533 | X65644 |
| Sequence 7534 | X66276 |
| Sequence 7535 | X67698 |
| Sequence 7536 | X69117 |
| Sequence 7537 | X70326 |
| Sequence 7538 | X73114 |
| Sequence 7539 | X73608 |
| Sequence 7540 | X73902 |
| Sequence 7541 | X74801 |
| Sequence 7542 | X75593 |
| Sequence 7543 | X75861 |
| Sequence 7544 | X76538 |
| Sequence 7545 | X79535 |
| Sequence 7546 | X80754 |
| Sequence 7547 | X80910 |
| Sequence 7548 | X80916 |
| Sequence 7549 | X81789 |
| Sequence 7550 | X81889 |
| Sequence 7551 | X81900 |
| Sequence 7552 | X83973 |
| Sequence 7553 | X84908 |
| Sequence 7554 | X85129 |
| Sequence 7555 | X85372 |
| Sequence 7556 | X87176 |
| Sequence 7557 | X93036 |
| Sequence 7558 | X94323 |
| Sequence 7559 | X97065 |
| Sequence 7560 | X98743 |
| Sequence 7561 | Y00281 |
| Sequence 7562 | Y00282 |
| Sequence 7563 | Y00503 |
| Sequence 7564 | Y08319 |
| Sequence 7565 | Y10313 |
| Sequence 7566 | Y13286 |
| Sequence 7567 | Z11241 |
| Sequence 7568 | Z14244 |
| Sequence 7569 | Z18538 |
| Sequence 7570 | Z26317 |
| Sequence 7571 | Z29330 |
| Sequence 7572 | Z47087 |
| Sequence 7573 | Z74615 |
| Sequence 7574 | N91258 |
| Sequence 7575 | N91825 |
| Sequence 7576 | N92483 |
| Sequence 7577 | Q13363 |
| Sequence 7578 | Q46089 |
| Sequence 7579 | Q65520 |
| Sequence 7580 | Q65676 |
| Sequence 7581 | Q75166 |
| Sequence 7582 | Q94780 |
| Sequence 7583 | T59274 |
| Sequence 7584 | V03517 |
| Sequence 7585 | V29653 |
| Sequence 7586 | V32447 |
| Sequence 7587 | V33197 |
| Sequence 7588 | V34263 |
| Sequence 7589 | V34271 |
| Sequence 7590 | V43612 |
| Sequence 7591 | V58360 |
| Sequence 7592 | V58628 |
| Sequence 7593 | V58648 |
| Sequence 7594 | V58684 |
| Sequence 7595 | V62427 |
| Sequence 7596 | V62428 |
| Sequence 7597 | V62430 |
| Sequence 7598 | V70354 |
| Sequence 7599 | V70355 |
| Sequence 7600 | V87930 |
| Sequence 7601 | X03841 |
| Sequence 7602 | X04316 |
| Sequence 7603 | X24060 |
| Sequence 7604 | X26850 |
| Sequence 7605 | X27262 |
| Sequence 7606 | X37486 |
| Sequence 7607 | X37532 |
| Sequence 7608 | X39868 |
| Sequence 7609 | X40372 |
| Sequence 7610 | X51668 |
| Sequence 7611 | X52235 |
| Sequence 7612 | X59352 |
| Sequence 7613 | X91484 |
| Sequence 7614 | Z13088 |
| Sequence 7615 | Z15085 |
| Sequence 7616 | Z15149 |
| Sequence 7617 | Z16695 |
| Sequence 7618 | Z18356 |
| Sequence 7619 | Z27252 |
| Sequence 7620 | Z33445 |
| Sequence 7621 | Z34217 |
| Sequence 7622 | AC28649 |
| Sequence 7623 | AC29170 |
| Sequence 7624 | AC29869 |
| Sequence 7625 | AC31081 |
| Sequence 7626 | AC31440 |
| Sequence 7627 | AC31443 |
| Sequence 7628 | AC32056 |
| Sequence 7629 | AC32097 |
| Sequence 7630 | AC32130 |
| Sequence 7631 | AC34175 |
| Sequence 7632 | AC34190 |
| Sequence 7633 | AC003991 |
| Sequence 7634 | AC004130 |
| Sequence 7635 | AC004223 |
| Sequence 7636 | AC004456 |
| Sequence 7637 | AC004499 |
| Sequence 7638 | AC005004 |
| Sequence 7639 | AC005037 |
| Sequence 7640 | AC006057 |
| Sequence 7641 | AC006257 |
| Sequence 7642 | AC006285 |
| Sequence 7643 | AF064859 |
| Sequence 7644 | AF103908 |
| Sequence 7645 | AL022097 |
| Sequence 7646 | AL031346 |
| Sequence 7647 | AL034555 |
| Sequence 7648 | AL079338 |
| Sequence 7649 | AP000527 |
| Sequence 7650 | S65371 |
| Sequence 7651 | U15177 |
| Sequence 7652 | Z83843 |
| Sequence 7653 | Z93020 |

TABLE 4-2
Sequence l949:found in patent publication WO99/46374
CCCTTAGCGTGGTCGCGGCCGAGGTACTTGGTGGACCACCATCACACCCTCCTGTGCAAT
GGGTATTGGCTTGCCTGGCTGATTCATGTGGGAGAGTCCTTGTATGCCATAGTATTGTGC
AAGCATAAAGGCATCACAAGTGGTCGGGCTCAGCTACTCTGGTTCCTACAGACTTTCTTC
TTTGGGATAGCGTCTCTCACCATCTTGATTGCTTACAAACGGAAGCGCCAAAAACAAACT
TGAAGTTGTCTGAAAGCTTGCTCTACACTTTTACATTCATCCTCACCCTTTTTTTTGTGG
GGTAGAGGAGGTGCAGTAATTTACTCAGTGATCTTTCTACTTTCTAGAAACTGTCCTTCA
AAGCTCTTTAAGACCCCCTCGTTAGTCAGTTTTTTCTCTTATATGCTCTGGTTGAGCTTG
AATAGACCAGTTGTTACTTAAGAAAGAAACAGAGAAAGATTTTAGCTTTTCAATCCTATT
TGGCAGAGGACTTCAGCTACCTTCTTACAGTCTTTGGCTGTGTTGGTACCTGCCCGGGCG
GCCGCTCGA
Sequence 1950:found in patent publication WO99/64576
GATATCTGCAGAATTCGCCCTTAGCGTGGTCGCGGCCGAGGTACATAAAGCAGATTCAAG
GGTTAAAATAAAAACAGAATTTTGGAGTGTGGTCAAATAAGGTGCACAGATTCCAGAACC
CTCAGAGGGCCTGCTGGCCCTCTCCAGACATTCTGTGTCCGTGGTGCAGGAGCTGGGCCC
GTCCCTAACAGCTCCGCACTGGCTTANTGCAGTGGTGCTCACAGTTTCAGGAACTACTAG
GTGAAGTGTCTGGCTCAAGTCTGCCAAGTGTCTTCACTCCATCGTCAGAAGTGGAGCACT
ATCCCTAGGTTCGATTCCCATGAAATATTTTATGATTTCCATCCTCTNTGCCCGCTCTTC
CAAATAAGGCCCTGTGATGCCAACGAAGGGGGCATGGTTGAGGGTCTAAGGCTCTCATTA
ANGGCCTAATTCTGTGTGGGATATCAACACATGACAGACACTTGACTGCAACATTCAAGA
CATTTAAGGCAGTGGGTTCATTTAATGACTACTTTTCCAAAATAAATA
Sequence 1951:found in patent publication W099/64594
CCCTTAGCGTGGTCGCGGCCGAGGTACCTCATTAGTAATTGTTTTGTTGTTTCATTTTTT
TCTAATGTCTCCCCTCTACCAGCTCACCTGAGATAACAGAATGAAAATGGAAGGACGGCC
AGATTTCTCCTTTGCTCTCTGCTCATTCTGTCTGAAGTCTAGGTTACCCATTTTGGGGAC
CCATTATAGGCAATAAACACAGTTCCCAAAGCATTTGGACAGTTTCTTGTTGTGTTTTAG
AATGGNTTTCCTTTTTCTTAGCCTTTTCCTGCAAAAGGCTCACTCAGTCCCTTGCTTGCT
CAATGGACTGGGCTCCCAGGGCCTANGCTGCCTTCTTTTCCATGTCCCACCCATGAGCCC
TCCACTGGACAGCTTANTAAGCCTGGCCCTTCATTCTGGGGCTGTGTTCTTTCTCTGGGA
AAATCAATACCTCTTACCTTCTNTTGCATGCAAAGATCTTAAAGGATTGTCAAACTTTCA
AAACGTTACAAGNAGAACCNNCCANNAAGGTCCTATAAAATGCCAGTAAGTGACCCTTNT
CAAGCTGTCAAGGCTTTTAAATTAGGANTTTGGGGATTTAAATGCTTTGTNTTTTTTTAA
AGGGAAANAAATAAGAGTTGCTNNNTTTTAAAAAATGCAATGTTTTTTTANCCAATTAAA
AATTTNNCCCCAAACTTTTTTTAAAAAGNAAANAAAAANACCNCTTTTGGGAGANCGGNA
AAAAAAAAAAA
Sequence 1952:found in patent publication W099/54461
CCCTTAGCGTGGTCGCGGCCGAGGTACAAGATGTGCATGCAGTCCAAGGCCATGAACGAG
GCATCCCACGGCCAGCTGGGCATGCTGGTGTTCAGGCACGAGATAGAGGCTCACCTTCGC
AAACAGAAGCAGAAGACAAGTAGCAAAAAAACATGAACTCCCAGAGAAGGATTGTGGGAG
ACACTTTTTCTTTCCTTTTGCAATTACTGAAAGTGGCTGCAACAGAGAAAAGACTTCCAT
AAAGGACGACAAAAGAATTGGACTGATGGGTCACAGATGAGAAAGCCTCCGATTTCTCTC
TGTTGGGCTTTTTACAACAGAAATCAAAATCTCCGCTTTGCCTGCAAAAGTAACCCAGTT
GCACCCTGTGAAGTGTCTGACAAAGGCAGAATGCTTGTGAGATTATAAGCCTAATGGTGT
GGAGGTTTTGATGGTGTTTACAATACACTNAGACCTGATGGTTTTGTGGNGCTCATTGAA
AATATTCATGAATTTAAAGAGCAGTTTTTGGTNA
Sequence 1953:found in patent publication W099/64594
CCCTTAGCGTGGTCGCGGCCGAGGTACTCTTTCTCTCCCCTCCTCTGAATTTAATTCTTT
CAACTTGCAATTTGCAAGGATTACACATTTCACTGTGATGTATATTGTGTTGCAAAAAAA
AAAAAGTGTCTTTGTTTAAAATTACTTGGTTTGTGAATCCATCTTGCTTTTTCCCCATTG
GAACTAGTCATTAACCCATCTCTGAACTGGTAGAAAAACATCTGAAGAGCTAGTCTATCA
GCATCTGACAGATGAAATTGGATGGTTCTCAGAACCATTTCACCCAGACAGCCTGTTTCT
ATCCTGTTTAATAAATTAATTTTTGGGGTTCCTCTACCATGCCATAGCAAAACCCCCTGC
TTCCAAATTCTTGTCAACAATTAAAAAGTCTGNTGGACCTTGGAAGTTT
Sequence 1954:found in patent publication W099/40189
TTTTCCNTTTTTTTTTTTTTTTTTTTTTTTTCCCCNCCAAGCTTTATCTAGNCTTAGACT
TTTTAAAAAAGTTTGGGGGCAGATTCTGAATTGGCTAAAAGACATGCATTTTTAAAACTA
GCAACTCTTATTTCTTTCCTTTAAAAATACATAGCATTAAATCCCAAATCCTATTTAAAG
ACCTGACAGCTTGAGAAGGTCACTACTGCATTTATAGGACCTTCTGGTGGTTCTGCTGTT
ACGTTTGAAGTCTGACAATCCTTGAGAATCTTTGCATGCAGAGGAGGNAAGAGGTATTGG
ATTTTCACAGAGGAAGAACACAGCGCAGAATGAAGGGCCAGGCTTACTGAGCTGNCCAGT
GGAGGGCTCATGGGNGGGACATGGAAAAGAAGGCAGCCTAGGCCCTGGGGAGCCCAGTCC
ACTGAGCAAGCAAGGGACTGAGTGAGCCTTTTGCAGGAAAGGGCTAANAAAAAGGNAAAC
CATTCTAAAACACANCAAGAAACTGGCCAAATGCTTTGGGAACTGGGGTTATTGGCCNAT
AATGGGGCCCCAA
Sequence 1955:found in patent publication W099/64594
CCCTTTCGAGCGGCCGCCCGGGCAGGTACTTTTTTTTTTTTTTTTTNTTTTTTTCCGCCT
CCCCAAAGCTTTATTTNTNTTGANTTTTTAAAAAAGTTTGGGGGCANATTCTGAATTGGC
TAAAANACATGCATTTTTAAAACTAGCAACTNTTATTTNTTTCCTTTAAAAATACATAGC
ATTAAATCCCAAATCCTATTTAAAGACCTGACAGNTTGANAAGGTCACTACTGCATTTAT
AGGACCTTCTGGNGGTTCTGCTGTTACGTTTGAAGTCTGACAATCCTTGANAATNTTTGC
ATGCANAGGAGGTAANAGGTATTGGATTTTCACAGAGGAAGAACACAGCGCANAATGAAG
GGCCAGGCTTACTGAGCTGTCCAGTGGAGGGCTCATGGGTGGGACATGGAAAANAAGGCA
GCCTAGGCCCTGGGGAGCCCAGTCCACTGAGCAAGCAAGGGACTGANTGAGCCTTTTGCA
GGAAANGGCTAANAAAAAGGAAAACCATTCTAAAACCCAACANGAAACTGTCCAAATGCT
TTGGGAACTGGGTTTATTGCCTATAAAGGGTCCCCAAAA
Sequence 1956:found in patent publication W099/64594
CTCGAGCCGGCTCGCCAGTGTGATGGGATATCTGCAGAATTCGCCCTTAGCCGTGGTCGC
GGCCCGATGTA
Sequence 1957:found in patent publication W099/64594
CCCTTAGCGTGGTCGCGGCCGAGGTACCTAGAAAACAGAAACTTGAGTAGACATGGTAAT
GACCAGAAAAGGCTATCTTTATACATTTCTTTTGCTACGCTTCAAATTCATGTCACCTAA
AAGTTGTGAAGTGCACAAAACAAATCTACTTAACTGAAAATTATTTTCAATGAATGGGAT
GTTTAGAACTCTGTGAGGGTTTTTAAGGTCTTTTCGAATAGCAAATTCTAATGAGGCTTT
TTTAAGTTGGCAATTTAAACTCATACAAGAAATAAAAACTCACCAGTGTGGCTGGGCAGA
ATATATATATTTTCTCAAATATTGTTTGTTTGTTTTTTCCCTGCACTGTATCCATGGTCC
CATGATGAAACTGTTATATTGCTGATATATTTATTGGAATATGTGGGCCAACTTCCTTTC
CACTCAACATATGGATTGGTAGTTTAAAATAATTCCTTTCTATTAAGCAAATGTGTGGCT
AAGGCACATTTAAATAGCCCATTAAACCAATGGAGATGACAATGTGTTACCCTCAGAGAA
AGCTTAATTTTT
Sequence 1958:found in patent publication W099/64594
CCCTTTCGAGCGGCCGCCCGGGCAGGTACTATATGGACACCAGCATGGAGCAGAAAATGA
GGGCAGCAGTGTCCAATCTCATTCCAGGTGAGAAGTTGCACAGTGTCCAATAGGTGCATA
CATCTCCTTAGTAAGTAGTTGTGATTAACAATGAAATAGAAATGAAAAATATATTTTTTT
ATTTATGTGTATTATATTTTTCAGGCAGCTAATAAGTTGGTAGGACATAATATTTAATTC
GTTGGGGACCTAATTATTTATAAATTGAATGGTTAGATATTTCTTTTGGCCTAAGCCACC
ATGAAAAAATCACTGAGGCGCTAAGGGAAACATGAACTAAGAAGCCCTTCTGAGTCTCTG
TTTTCTCAACTGTAAGATGAAGAAACCTGCTCCACCTTCCTCTTGCGATTGTCGAACGGC
AGTTGAGATACTGCGCCAGAATGGACCCTTATTGATGGCCTAC
Sequence 1959:found in patent publication W099/63088
CCCTTTCGAGCGGCCGCCCGGGCAGGTACCTCATTAGTAATTGTTTTGTTGTTTCATTTT
TTTCTAATGTCTCCCCTCTACCAGCTCACCTGAGATAACAGAATGAAAATGGAAGGACAG
CCAGATTTCTCCTTTGCTCTCTGCTCATTCTCTCTGAAGTCTAGGTTACCCATTTTGGGG
ACCCATTATAGGCAATAAACACAGTTCCCAAAGCATTTGGACAGTTTCTTGTTGTGTTTT
AGAATGGTTTTCCTTTTTCTTAGCCTTTTCCTGCAAAAGGCTCACTCAGTCCCTTGCTTG
CTCAAGTGGACTGGGCTCCCCAGGGCCTAGGCTGCCTTCTTTTCCATGTCCACCCATGAG
CCCTCCACTGGACAAGCTCAGTAAGCCTGGCCCTTCATTCTGCGCTGNGTTCTTCTCTGT
GAAAATCCAATACCTCTTACCTTCTCTGCATGCAAAGATTCTCAAGGATTGCAGACTTCA
AACGTAACAGCAGAACCACCAGAAGGTCCTATAAATGCAGTAGTGACCTTTTCAAGCTGT
CAGGCCTTTAAATANGAATTTGGGAATTTAATGCTATGTATTTTTNAAAGGAAAGAAATA
AGAAGTTGCTTAGNTTTNAAAATGCATGTCTTTTANCCCAATTAAAAATTTGCCCCCAAA
CTT
Sequence 1960:found in patent publication W099/64594
NGATATCTGCANAATTCGCCCTTTCGAGCGGCCGCCCGGGCAGGTACAACTTTTTAAATA
GGGAATATGATAGCTTNGCATGGTGGTGTGCACCTATAGCCCCCACTGCCTGGAAAGCTG
GGGTGGGAGAATCGCTTGAGTCCAGGAGTTTGAGGTTACAGTGATCCACGATCNNGCCAC
TACACTCCAGCCTGGGCANCAGAGCAAGACCCTGTNTCAAAGCATAAAATGGAATNACAT
ATCAAATGAAACANGGAAAATGAAGCTGA
Sequence 1961:found in patent publication W099/64584
CCCTTTCGAGCGGCCGCCCGGGCAGGTACCTTTTTTTTTTTTTTTTTTTTTCCGTCTCCC
CAAAGCTTTATCTGTCTTGACTTTTTAAAAAAGTTNGGGGGCANATTNTGAATTGGNTAA
AAGACATGCATTTTTAAAACTAGCAACTCTTATTTCTTTCCTTTAAAAATACATAGCATT
AAATCCCAAATCCT
Sequence 1962:found in patent publication W099/64594
GCATGCTCGAGCCGGCCGCCAGTGTGATGGGATATCTGCAGAATTCGCCCTTTCGAGCGG
CCCGCCCGG
Sequence 1963:found in patent publication W099/64594
CCCTTTCGAGCGGCCGCCCGGGCAGGTACAAGCTATCTTTTGCTCCAAAACAGTTCTGAA
GGTTTTATTTATATTTTATCTTATCCCGAGGGACCAACAGCAGGCATACCTTTGCCAGGC
CTTCTTGCAGAAAGACACAGAGCCGTAAAGGCAAAAATAAAATTGCAATAAAGTATATGG
TATTGGGGGCAGGGAGAACCAGAAACCCTCAAAGAGAACCAATTTGTAGCACGTTCTTTT
TTAAGGCTCTACCCCTGTAGAAGTAAGAAACTAGCCTGCCTTTTTAGCCATATGAGAGTT
TCCTCCAGAGCCATCTTCCAAAGTAGCAGACTTGGCCAAGTTGCCCAATGCCAAATAAGT
GAGTTGGAAGTTCGTTTGCTTCAAACACACTGCACTTAGAAACCAGACTTGAAATAATCG
AAGCCCCACAGAAAAGCTTCATGAAACGAAGTGTTACTTTCCTAGAGAATAAGAAAGTCA
CAAGATTGAGGAGTCTGTTCTAAAGTTCT
Sequence 1964:found in patent publication W099/64594
CCCTTTCGAGCGGCCGCCCGGGCAGGTACCTCATTAGTAATTGTTTTGTTGTTTCATTTT
TTTCTAATGTCTCCCCTCTACCAGCTCACCTGAGATAACAGAATGAAAATGGAAGGACAG
CCAGATTTCTCCTTTGCTCTCTGATCATTCTCTCTGAAGTCTAGGTTACCCATTTTGGGG
ACCCATTATAGGCAATAAACACAGTTCCCAAAGCATTTGGACAGTTTCTTGTTGTGTTTT
AGAATGGTTTTCCTTTTTCTTAGCCTTTTCCTGCAAAAGGCTCACTCAGTCCCTTGCTTG
CTCAGTGGACTGGGCTCCCCAGGGCCTAGGCTGCCTTCTTTTCCATGTCCACCCATGAGC
CCTCCACTGGACAAGCTCAGTAAGCCTGGCCCTTCATTCTGCGCTGTGTTCTTCCTCTGT
GAAAATCCAATACCTCTTACCTCCTCTGCATGCAAAGATTCTCAAGGACTGGCAGACTTC
AAACGTAACAGCAGAACCACCAGAAAGGCCTATAAATGCAGTAGTGACCTTCTCAAGCTG
CANGTCTTTAAATAGGATTTGGGAATTAATGCTATGTANTTTTAAANGGAAAGAAATAAG
AAGTTGCTAGTTTTAAAAATGCATGTTTTTAAGCCAATTCAAAAATTGGCCCCAACTTTT
TTTAAAAAGTCAAGACAAGATAAAGCNTTTGGGGAGAACGGAAAAAAAAAAAAAAA
Sequence 3723:found in patent application W099/57132
CCCTTAGCGTGGTCGCGGCCGAGGTACTCATGTATTTTTTTTTTCCAGATCTCTTTCCCC
AAGTTGCTATTGTAAGAGTATTCTGCTGCGTGTGGATGCAGTTATACACATTAAAGCAGA
TCTGGAGTCTGAAGTAGCTATAAAGCAGCTATAAAACAGAAATACATGCATAGCTGCAGA
AACCATGATAGGTAGAGGACTTTTCTTTTGGTTTTGTTTTGTTTTGTTTTGTTTTGTTTT
TGGTTTTACAGAGAAGAGATTTTTATTCAAAGAAAAAAATTCCAGTGAATTGTGCACAAA
TGCTGGTTTTTACACCATCCTAAAGAAAAACTTTACAAGGGGTGTTTTGGAGTANAAAAA
AGGTTATAAAGTTGGAATCTTAAATTGTNAAATTAACCATTGAGTGTCAAGGNTCTAAAA
GCAGAACTTATTTTGTGCAATGAACATAANGAAAGACTACTGTATAGGGTTTTTTTT
Sequence 3724:found in patent application W099/64594
CCCTTTCGAGCGGCCGCCCGGGCAGGTACTCAGACAAATACAACGTGCCCATTTCGTCAG
ATATTGCGCAGAACCAAGAATTTTATAAGAACGCAGAAGTTAGACCACCATTTACCGTAT
GCATNTTTAATTAAGGCAGGCCATTCTCGAATCTCCAGAAAAGCAGCTAACACTAAATGA
GATCTATAACTGGTTCACACGAATGTTTGCTTACTTCCGACGCAACGCGGCCACGTGGAA
GAATGCAGTGCGTCATAATCTTAGTCTTCACAAGTGTTTTGTGGCGAGTAGAAAACGTTA
AAGGGGGCAGTATGGGACAGTGGATTGAAGTANGAATTCCAAAAACGAAGGGGCCAAAAA
GGATCAGTGGGTAACCCTTT
Sequence 3725:found in patent application W099/64594
NTNTTAGCGTGGTCGCGGCCGAGGTACTTTTTGCTTTANCAGATAGATAGGGCATCCAAT
ACAACTGAAACAACCTGATAACAAATTAATTTTATTTTTCAATAAAAAGGAATGCTCTGG
TTTTTTAACTGGCTCCTTGAGGAAGCCAGAAGATGGCATCTGCTCTTTAAACAGGCTTCT
CTTATCTGATTTGAGACACAAATCCACCAAGNATTCTTTCTTACAGTGGAAGTAGGCACC
ACCCCTCAGATCACGAGGCCCCTAGGNAAGAAGCCTGTGGGAAATCAGCCCTGTGATGTT
GTGGGTGGGTGGGCCAGCACCTAGAAGACACATGGTNGGTTGTGCAACATAAAATCCCCT
TCAAGCGGACTCAGTGG
Sequence 3726:found in patent application W099/64594
CCCTTAGCGTGGTCGCGGCCGAGGTACTCTGGTCATCTTCGTTCGTTTGGTCGTGCAAGG
TGTTAACTATTTTCACTTCCCATATCACAAAGTTAGTCCACAGGAGGAGCTCGTGGATCT
TGTCCATTATGAGGACTGGTTGGCTTTCCAGGTAGGCTGGATCCTCTTAGATTAGGAGGG
TCTCAGTAAAGAACAAGATCAATGCAANTAACCATCCAGGCTACCAAGATCATTGTTAAC
ACTGATGCCATTATCACCAGAGGGTCCCGGTAAATTCCTTGAAGACACTCTGTGTCTGTT
GCANGTANGACTGGGACTNCCGTAACAGATTGATCAAGNTCTTCTCANTTGCATGTTCAT
GGAGAGCAAACACATTCAC
Sequence 3727:found in patent application W099/64594
CCCTTAGCGTGGTCGCGGCCGAGGTCGGCCGAGGTACTTTATTTTCAAAACACTCATATG
TTGCAAAAAACACATAGAAAAATAAAGTTTGGTGGAGGTGCTGACTAAACTTCAAGTCAC
AGACTTTTATGTGACAGATTGGAGCAGGGTTTGTTATGCATGTAGAGAACCCAAACTAAT
TTATTAAACAGGATTAGAAACAGGCTGTCTGGGTGAAATGGTTCTGAGAAGCATCCAATT
CACCTGTCAGATGCTGATAGACTAGCTCTTTCAGATGTTTTTTTCTACCAGTTCAGAAGA
TNGGGTTTAAATNGACCTAGTTCCAATGGGGGAAAAAAGCAAGAATGGGATTTCACAAAA
CCCAAGGTNATTTTTAAACCAAAAGACCCCTTTTTTTTTGGCA
Sequence 3728:found in patent application W099/55858
CCCTTAGCGTGGTCGCGGCCGAGGTACCATGCTGACTTCTTGGTATCTTTTAAGGCCTAA
TTTTCCCTTCCTTGAGATTACTGTAGTGTGTTCCAGCTAATTTCTATTTGGAAACGAGTT
GGAACAGCTGAAAACTGGGTATTATTGAAGGCAAAGCAGCCTCACGTCAGTTTTTTATCA
GCTCATTTGGGAAGTTTTTTTTTTTTTTTTTTTAATTAATTAGAAAGTAGGCTGGGCACG
GTGGCTCATGCCTATAATCCCAGCACTTGGGGAGGCCGAGGATCTCCTCTCTGGTGGATC
ACTTGAGGGCAGGAGTTAAGAGACCATCCTGGCCAACATGATGAAACCCTGTCTCTACTA
AAAATACAAAAAGTAGCT
Sequence 3729:found in patent application W099/64594
CCCTTAGCGTGGTCGCGGCCGAGGTACAGAATTTATTATGAAATAGCTTAATGGCAAGTG
GTAATTTAGAAGAATTAAGTTATCAGATAGGAGATATATTAAAATATTTAAAAATTGGAT
ATATTCTTGAAGCCCTTTTACACAAGTAATTTCTATAATTTGATTGTAATGAAAGTATAA
TATACCTTGTTACTATTATCAGGATTAATTTTTGAAAGTAGAATTGCTTAATCAAGCCAA
GGTTAATGCTGCTTTATAGGAAATTAATCAGGTAGTTTAACACTAGAGCTCATTAGCCCA
ACCTGTATGTAGCACAAAAATAAATCATTCTCTGATAAATNCCNTATTAAAATANTATTT
TTTAATTCATACCTTTTTAAAATAA
Sequence 3730:found in patent application W099/57l32
CCCTTAGCGTGGTCGCGGCCGAGGTACTTTTTTTTTTTTTTTTTTTTTTTCCGTCTCCCC
AAAGCTTTATCTGTCTTGACTTTTTAAAAAAGTTTGGGGGCAGATTCTGAATTGGCTAAA
AGACATGCATTTTTAAAACTAGCAACTCTTATTTCTTTCCTTTAAAAATACATAGCATTA
AATCCCAAATCCTATTTAAAGACCTGACAGCTTGAGAAGGTCACTACTGCATTTATAGGA
CCTTCTGGTGGTTCTGCTGTTACGTTTGAAGTCTGACAATCCTTGANAATCTTTGCATGC
AGAGGAGGTAAGAGGTATTGGATTTTCACAGAGGAAGAACACANCGCAAAATGAAGGGCC
AGGCTTACTGAGCTGTCCAGTGGAGGGCTCATGGGTGGGACATGGAAAAGAAGGCAGCCT
AGGCCCTGGGGAGCCCANTCCACTGAGCAAGCAAGGGACTGAGTGAGCCTTTNCAGGAAA
AGGCTAANAAAAAGGAAAACCATTTTAAAACACAACAAGAAACTTGTCCAAATGCTTTGG
GAACCGNGTTTATTGCCTATAATGGGTCCCCCAAAATGGGTAACCTAAACTTTAAAAGAN
AATGAACCCNANAGCNNAAGGAAAAATCTGGCTTGCCCTTCCATTTTCATTCTNTTATNT
TAAGGNGACCTTTTTNANGGGGANCCTTTTN
Sequence 373l:found in patent application W099/64594
CCCTTAGCGTGGTCGCGGCCGAGGTACTTTTTTTTTTTTTTTTTTTTTCCGTCTCCCCAA
AGCTTTATCTGTCTTGACTTTTTAAAAAAGTTTGGGGGCAGATTCTGAATTGGCTAAAAG
ACATGCATTTTTAAAACTAGCAACTCTTATTTCTTTCCTTTAAAAATACATAGCATTAAA
TCCCAAATCCTATTTAAAGACCTGACAGCTTGAGAAGGTCACTACTGCATTTATAGGACC
TTCTGGTGGTTCTGCTGTTACGTTTGAAGTCTGACAATCCTTGAGAATCTTTGCATGCAG
AGGAGGTAAGAGGTATTGGATTTTCACAGAGGAAGAACACAGCGCAGGATGAAGGGCCAG
GCTTACTGAGCTGTCCAGTGGAGGGCTCATGGGTGGGACATGGAAAAGAAAGGCAGCCTA
AGCCCTGGGGAGCCCAATCCGCTGAGCAAGCAAGGGACTGANTGAGCCTTTTGCAGGAAA
AGGCTTANAAAAANGAAAACCATTNTTAAAAACAACAAGAAACTTTTCCAAATGCTTTNG
GAACCGGGGTTATTGGCCATAAATGGGNCCCCCAAATGGGTAANCCAAACTTTAAAAAAN
ATGANCCGAAANCAAAGGGNAAAATCTGGGTGGCCCTTCCATTTNATTNTNGTANNNCAA
AGGGAACCTGGNNNAAGGGGGGNCCTTTTNAAAAAAA
Sequence 3732:found in patent application W099/64594
CCCTTAGCGTGGTCGCGGCCCGAGGTACTTTTTTTTTTTTTTTTTTTTTTTTCCCGCCTC
CCCAAAGCTTTATCTGTCTTGACTTTTTAAAAAAGTTTGGGGGCAGATTCTGAATTGGCT
AAAAGACATGCATTTTTAAAACTAGCAGCTCTTATTTCTTTCCTTTAAAAATACATAGCA
TTAAATCCCAAATCCTATTTAAAGACCTGACAGCTTGAGAAGGTCACTACTGCATTTATA
GGACCTTCTGGTGGCTCTGCTGTTACGTTTGAAGTCTGACAATCCTTGAGAATCTTTGCA
TGCAGAGGAGGTAAGAGGTATTGGATTTTCACAGAGGAAGAAGACAGCGCANAATGAAGG
GCCAGGCTTACTGAGCTNTCCAGTGGAGGGCTCATGGGTGGGACATGGAAAANAAGGCAG
CCTANGCCCTGGGGAGCCCANTCCACTGAGCAAGCAAGGGACTGANTGAGCCTTTTGCAG
GAAAAGGCTTAAAAAAAGGAAAACCATTCTAAAACACAACAAGGAAACTGTCCAAATGCT
TTGGGAACTGGGTTTATTTGCCTATTATGGGGTCCCCAAAAA
Sequence 3733:found in patent application W098/39448
CCCTTAGCGTGGTCGCGGCCGAGGTACTACAAAAACAGAATAATTTTGAAGTTTTAGAAT
AAATGTAATATATTTACTATAATTCTAAATGTTTAAATGCTTTTCTAAAAATGCAAAACT
ATGATGTTTAGTTGCTTTATTTTACCTCTATGTGATTATTTTTCTTAATTGTTATTTTTT
ATAATCATTATTTTTCTGAACCATTCTTCTGGCCTCAGAAGTAGGACTGAATTCTACTAT
TGCTAGGTGTGAGAAAGTGGTGGTGAGAACCTTAGAGCAGTGGAGATTTGCTACCTGGTC
TGTGTTTTGAGAAGTGCCCCTTAGAAAGTTAAAAGAATGTAGAAAAGATACTCAGTCTTA
ATCCTATGCAAAAAAAAAATCAAGTAATTGTTTTCCTATGAGGAAAATAACCATGAGCTG
TATCATGCTACTTAGCTTTTATGTAAATATTTCTTATGTCTCCTCTATTAAGAGTATTTA
AAATCATATTTAAATATGAATCTATTCATGCTAACATTATTTTTCAAAACATACATGGAA
ATTTAACCCAATTGTCTCATATAAGGTTTTATTTGAATTG
Sequence 4144:found in patent application W099/64594
CCCTTTCGGCGGCCCGCCCGGGCAGGTACCTGGGGTCTCAGGGTTGCTCTGGGCCTGATC
ATCCACTCAGATCTGTAAGGAGGATTTGCAGGATCCATTTAGAAAGATCCTCCCTTACTT
CCACAAGCATGGCCTTTGGCTCTTAAATACCTGTGCTGGGGTTTTGTAATTATAGAAACA
ACAGGAACCAAAACTCATTAATGTTGAGCTACAAACCAGAGGGAAGCTTCTTTCTCAAAA
CAGGGCTCAGGCCTAGAAAAATCTAGTTTTCTGAAATCGCTAGCCAGCAACAGCACTGAG
ATGGCCATCCCAGAAACAAGGCCAACACAGAAGCACCCATAAAGGCCGCTGGAGGTTGGG
ACAAAGAGATGCTTGCTGTCCTTACAGACCCCCTGACTTCCAAGGAGCTCCCCTCTTACC
CAGCCTGGCCTGCCTTCTCCACAGGGTAGCTGATCGTCAGCATCATCTTCAATGGTGTTG
CCAAAAGCACTCAANTGCTCCTGCCATCCCTGTCCATCTTCAACATGAAAAGGAGAGGTT
AGCACTTCACCTGGGCGACTGAGCAAGANTTCATCTTAAAAAAAAAAAAAAATT
Sequence 4145:found in Patent application W099/64594
CCCTTAGCGTGGTCGCGGCCGCCCGGGCAGGTACTTTTTTTTTTTTTTTTTTTTTTTACC
GGCTCCCCAAAGCTTTATCTGTCTTGACTTTTTAAAAAAGTTTGGGGGCANATTCTGAAT
TGGCTAAAAGACATGCATTTTTAAAACTAGCAACTCTTATTTCTTTCCTTTAAAAATAGA
TAGCATTAAATCCCAAATCCTATTTAAAG
Sequence 4146:found in Patent application W099/64594
ATCTGCAGAATTCGCCCTTANCGTGGTCGCGGCCGAGGTACTTTTT
Sequence 4l47:found Patent application W099/63088
CCCTTAGCGTGGTCGCGGCCCGAGGNACCATGATATCATGTATCCTGCTTGGACATTNTG
GGAAAGGGGGACCAGCTGGTTGGCCAATTTATCCTACAGGTCTTGGACGGNGGGACCTCT
TCAAAAGAAGATCTGGTAAGGGCAAGCAGCACAAGTGGCCATGGAAAAAGAAAAACTCTA
CANNATATTTCCGAGGATCAAGGACAAGTCCAAAACGAGATCCTCTCATTCTTCTGTCTC
GGAAAAACCCAAAACTTGTTTGATGCAGAATACACCAAAAACCAGCCTGGAAATCTATGA
AAGATCCTTAGGAAANGCCAGCTGCTAAGGATGTCCATCTTGTGGATCACTGCAAATACA
AGTATCTGTTTAATTTTCGAGGCGTAACTGCAAGTTTTCCGGTTAAACACCTCTTCCTGT
GTGGCTCACTTGGTTTCCATGTTNGGTGATGAAGTGGCTAGAATTCTTCTATCCACAGCT
TGAAGCCNTGGGTTCACTATATTCCAAGTCAAAACAGATCTCTTCAATGTCCAANAGCTG
TTACAATTTTGAAAAACCAAATGATGATGTAACTTCAAGAAAATTGCTTGAAAGGGGGAA
G
Sequence 4l48:found Patent application W099/64594
CCCTTAGCGTGGTCGCGGCCGAGGTACCACAAAAACAGAATAATTTTGAAGTTTTAGAAT
AAATGTAATATATTTACTATAATTCTAAATGTTTAAATGCTTTTCTAAAAATGCAAAACT
ATGATGTTTAGTTGCTTTATTTTACCTCTATGTGATTATTTTTCTTAATTGTTATTTTTT
ATAATCATTATTTTTCTGAACCATTCTTCTGGCCTCAGAAGTAGGACTGAATTCTACTAT
TGCTAGGTGTGAGAAAGTGGTGGTGAGAACCTTAGAGCAGTGGAGATTTGCTACCTGGTC
TGTGTTTTGAGAAGTGCCCCTTAGAAAGTTAAAAGAATGTAGAAAAGATCTCAGTCTTAA
TCCTATGCAAAAAAAAAAATCAAGTAATTGTTTTCCTATGAGGAAAATAACCATGAGCTG
TATCATGCTACTTAGCTTTTATGTAAATATTTCTTATGNCTCCTCTATTAAGAAGTATTT
AAAATCATATTTAAATATGAATCTATTCATGCTAACATTATTTTCAAAACATACCTGGAA
ATTAACCCAGAATGGCTACATATAAGGGTT
Sequence 4149:found Patent application W099/64594
CCCTTTCGAGCGGCCGCCCGGGCAGGTACCTCATTAGTAATTGTTTTGTTGCTTCATTTT
TTTCTAATGTCTCCCCTCTACCAGCTCACCTGAGATAACAGAATGAAAATGGAAGGACAG
CCAGATTTCTCCTTTGCTCTCTGCTCATTCTCTCTGAAGTCTAGGTTACCCATTTTGGGG
ACCCATTATAGGCAATAAACACAGTTCCCAAAGCATTTGGACAGTTTCTTGTTGTGTTTT
AGAATGGTTTTCCTTTTTCTTAGCTTTTTCCTGCAAAAGGCTCACTCAAGTCCCTTGCTT
GCTCAGTGGACTGGGCTCCCCAGGGCCTAGGCTGCCTTCTTTTCCATGTCCCACCCATGA
GCCCTCCACTGGACAGCTCAGTAAGCCTGACCCTTCATTCTGCGCTGTGTTCTTCCTCTG
TGAAAATCCAATACCTCTTACCTCCTCTGCATGCAAAGATTCTCAAGGATTGTCAGACTT
CAAACGNGACAGCAGAACCACCAGAAGGTCCTATAAATGCAGTAAGTGACCTTCTCAAGC
TGTCAGGTCTTTAAATAGGATTTGGGATTTAATGCTATGTATTTTTAAAGGAAAGAAATA
AGAAGTTGCTAGTTTTAAAAATGCATGTCTTTTAGCCAATTTAGAATCTGGCCCCAAACT
TTTTTAAAAAGTCAAAGACAGATAAAGCTTTNGGGAGACGGAAAAAAAAAAAAAAAAAAA
AAGT
Sequence 4150:found Patent application W099/64594
CGCCAGTGTGATGGGATATCTGCAGAATTCGCCCTTTCGCGGCCGCCCGGG
Sequence 4151:found Patent application W099/64576
CCCTTTCGAGCGGCCGCCCGGGCAGGTACAGTCATCCCACTACCTGGCTATTTCATTACT
TGGTGCTCTAGACAAGCTCCAAGAACTGACTGGATCTTGGCTTGCTCTGTTTCTGTCATT
GCTAATATAATATGGAAAACATTGCTGAAAAGAACAGAGATGGCCATGGATATGGCTAGG
TTAGGTATTCATATCCAAATATCTGAACTCTAACCTAATGTGGATATGATTCTGTAGCAT
TATATTAAAAGCTATGATGATGCAATGCAGGAAATAACCTTTCATTCTCCCCCCTAGAGG
ATCACGACAGGTGCTTTCAATGCCTGCCTTATCTATGGGACAAGTAGTGTGATTCTTCAA
GTGAGAAGTGAAAGCCTTTGGGGGATTTGAGTCANGAAGGGGAACATGGCTAAATTGCCT
GGAAACTCTGCCAACAAGTCTGCGGGTAGATTCTACTTGTCTCTGGGATAAAAAAATCTG
TGCTCAATGAAACTTATTGTGTTTGGAAAA
Sequence 4152:found Patent application W099/64594
TGCTCGAGCCGGCCCGCCAGTGCGANGGATATCNGCACAATTCCCCCTTTCAAGCGGCC
Sequence 5614:found in patent application W099/46375
CCGCGGTGGCGGCCGAGGTACCACAAGATATTATGCATGCTCCAATCGAGCAAAATGTCC
ATCATAACAAATATAAAATGTTTCCAGAAAGTATCATAGCGTGGTTTCCCATCTCCTGTA
TGGCTTAGGCTCCAAGGTCTGTGAGTTAATGCTGTTATTACATCCCGATCTGTTTGTCTC
ATCTTGTAGATAACTTCTGGCAAGAAAGAACAGACCACACTATTATTATACAGTTGAGGA
AGCTCTAGAACTAGN
Sequence 5865:found Patent application W099/64594
CCGCGGTGGCGGCCGAGGTACCTCATTAGTAATTGTTTTGTTGTTTCATTTTTTTCTAAT
GTCTCCCCTCTACCAGCTCACCTGAGATAACAGAATGAAAATGGAAGGACAGCCAGATTT
CTCCTTTGCTCTGTGCTCATTCTCTCTGAAGTCTAGGTTAGCCATTTTGGGGACCCATTA
TAGGCAATAAACACAGTTCCCAAAGCATTTGGACAGTTTCTTGTTGTGTTTTAGAATGGT
TTTCCTTTTTCTTAGCCTTTTCCTGCAAAAGGCTCACTCAGTCCCTTGCTTGCTCAAGTG
GACTGGGCTCCCCAGGGCCTAGGCTGCCTTCTTTTCCATGTCCCACCCATGAGCCCTCCA
CTGGACAGCTCAGTAAGCCTGGCCCTTCATTCTGCGCTGTGTTCTTCCTCTGTGAAAATC
CAATACCTCTTACNTCCTCTGCATGCAAAGATTCTCAAGGATTGTCAGACTTCAAAACGT
NACAGCAGAACCACCAGAAAGGTCCTATAAAATGCAGNTAGTGGACCTTCTTCAAGCTGT
CAGGGTCTTTTAAATAGGATTTGGGGGATATAATGGCTATTGTATTTTTAAAAAGGGAAA
GNAAATNAAGAGTTTGCTAGATTCTTAAAAAANGCCATGGTCTTTTTANCCAATTTNAAA
AATNTTGCCCCCCAACTTTTTTT
Sequence 7622:found in patent application W099/46374
TCGAGCGGCCCGCCCGGGCAGGTACTTTTTTTTTTTTTTTTTTGGGATTTTTTAGGTAGT
GGGTGTTGAGCTTG
Sequence 7623:found in patent application W099/51732
CGAGGTACGTTCTTCTTTCCCAGTTTCTCTACCCTTAGCTTATCCTCACCCTCATTTTGC
CCTGCTGGCTGCTCAAACTATGCAACAGATTCGGCATCCTCGCTTACCCATGGCCCAGTT
TGGAGGAACCTTCTCACCTTCTCCTAACACATGGGGACCATTCCCAGTGAGACCTGTGAA
TCCTGGCAACACAAATAGCTCTCCAAAGCATAATAACACAAGCCGTCTACCTAACCAGAA
CGGGACTGTTTTACCCTCAGAGTCTGCTGGACTAGCTACTGCCAGTTGTCCTATCACTGT
CTCTTCTGTAGTTGCTGCCAGTCAGCAACTGTGTGTCACTAATACCCGGACTCCTTCATC
AGTCAGAAGCAGTTGTTTGCCTGTGTGCCTAAGACAAGTCCTCCAGCAACAGTGATTTC
TTCTGTGACAAGCACTTGTAGTTCCCTGCCTTCTGTCTCCTCTGCACCTATCACTAGCGG
GCAAGCTCCCACCACATTTCTACCTGCAAGTACCTGCCCG
Sequence 7624:found in patent application W099/42470
CCGCGGTGGCGGCCGCCCGGGCAGGTACCGATACAGAAGAAGCTGCGTTTTGAAGACACC
CTGGAGTTTGTAGGGTTTGATGCGAAGATGGCTGAGGAATCCTCCTCCTCCTCCTCCTCA
TCTTCACCAACTGCTGCAACATCTCAGCAGCAGCAACTTAAAAATAAGAGTATATTAATC
TCTTCTGTGGCTTCGGTGCATCATGCAAACGGCCTAGCCAAATCTTCTACCACCGTCTCT
AGCTTTGCTAACAGCAAACCTGGCTCTGCTAAGAAGTTAGTGATCAAGAACTTTAAAGAT
AAGCCTAAATTACCAGAAAACTACACAGATGAAACCTGGCAAAAACTGAAAGAAGCAGTG
GAAGCTATTCANAATAGTACCT
Sequence 7625:found in patent application W099/47669
CCGCGGTGGCGGCCGAGGTCTATATGGACCACNAACATGGGAGCAGAAAAATGAGGGCAG
CAGTGTCCAATCTCATTCCAGGTGAGAAGTTGCACAGTGTCCAATAGGTGCATACATCTC
CTTAGTAAGTAGTTGTGATTAACAATGAAATAGAAATGAAAAATATATTTTTTTATTTAT
GTGTATTATATTTTTCAAGCAGCTAATAAGTTGGTAGGACATAATATTTAATTCGTTGGG
GACCTAATTATTTATAAATTGAATGGTTAGATATTTCTTTTGGCCTAAGCCACCATGAAA
AAATCACTGAGGCGCTAAGGGAAACATTGAACTAAGAAGCCCTTCTGAGTCTCTGTTTTC
TCAACTGTAAGATGAAGAAACCTGCTCCACCTTCCT
Sequence 7626:found in patent application W099/57132
CGCCCGGGGCAGGTACCTTAGTGAGGCTCAAAAGGATTCTTTTGGGTCTATTTTACGCCT
TATCTTTGAAATTCACCACTCGGGTGAGAAAGGTGACATTGTAGTCTTTCTGGCCTGTGA
ACAAGATATTGAGAAAGTCTGTGAAACTGTCTATCAAGGATCTAACCTAAACCCAGATCT
TGGAGAACTGGTGGTTGTTCCTTTGTATCCAAAAGAGAAATGTTCATTGTTCAAGCCACT
CGATGAAACAGAAAAAAGATGCCAAGTTTATCAAAGAAGAGTGGTGTTAACTACTAGCTC
TGGAGAGTTTTTGATCTGGAGCAACTCAGTCAGATTTGTTATCGATGTGGGTGTGGAAAA
GAAAAAAGGTGTCCTCGGC
Sequence 7627:found in patent application W099/l8208
CGAGGTACCTGTATGAGGTCTCCCAGCCCGAAGGACTAGGCCAAGCCCTCTGTGTGCCAT
CTCCAATGAGAAGGAATCCTGCCCTCACCTCACCCTTTTCCAACTTGCCCAGGGAAGTGG
AGGTTCCCTCTTTCCTTTCCTCTTGCAGGCCATCCATGACTTTAGAGAACAGACACAAGT
GTATCCAGCTGTCCACGGGTGGAGCTACCCGTTGGGCTTATGAGTGACCTGGAGTGACAG
CTGAGTCACCCTGGGTAAGTTCTCAGAGTGGTCAGGATGGCTTGACCTGCAGAAGATACC
CAAGGTCCAAAAGCACGAAGGTCTGCGGAAAGTTCTGGTTGTCGGCTGGCACCACGGTTA
CACCTATAATCGAGCACTTTGGGAGGCCAAGACAGGAGGATCGCTTGAGACCAAGAGTTT
GAGCCTGCGGTGAAGCTGTGAATGCACCACGGCACTCAAGCCTGGCAATGTAGCAAGATC
CTGTCTCTACAAGAAAATTTTTTAAAAATGAGCCAAGTGTGGGGGTGCATGCCTGTAGTT
TCCAGCTACTTNAGGACACTTACNTANGANGGTTGGTTTGAAACTGAAAAGTTGGAGGCT
TGCAATNANCCTTGAATGCCCCANTGGCCTTCAACCTGGGGCGAAAANAACCAAGACCCC
ATNTTAAAAAAAAAAAAANTTNTNGGGTNGNNATTGAATTGGGAAAAAAAAAAAGCTTGG
AGCTTTTTGCCTTTNGGCCNAGGNGANCATTGNAATTTGGGATTTTNGAANGGAATGGG
Sequence 7628:found in patent application W099/40189
CCCTTAGCGTGGTCGCGGCCGAGGTACCCGTTGCTGCTGCCATGTGTGTGCTTAAAACAG
GGTTCCTTTTTGTAGCATCAGAATTTGGAAACCATTACTTATATCAAATTGCACATCTTG
GAGATGATGATGAAGAACCTGAGTTTTCATCAGCCATGCCTCTGGAAGAAGGAGACACAT
TCTTTTTTCAGCCAAGACCACTTAAAAACCTTGTGCTGGTTGATGAGTTGGACAGCCTCT
CTCCCATTCTGTTTTGCCAGATAGCTGATCTGGCCAATGAAGATACTCCACAGTTGTATG
TGGCCTGTGGTAGGGGACCCCGATCATCTCTGAGAGTCCTAAGACATGGACTTGAGGTGT
CAGAAATGGCTGTTTCTGAGCTACCTGGTAACCCCAACGCTGTCTGGACAGTGCGTCGAC
ACATTGAAGATGAGTTTGATGCCTACATCATTGTGTCTTTCGTGAATGCCACCCTAGTGT
TGGCCATTGGAGAAACTGTAGAAGAAGTGACTGACTCTNGGTTCCTGGGGACCAC
Sequence 7629:found in patent application W099/47669
AGGTACTCTCGTTTCAGCTGGGCTCTTATGGCCAACCGCTCGGCTTGCGCCCGCCGGGTT
TCCGGANTATATGTTGTATTCGGCTGGGTCGAGGGTCTCAGGCAGAGTGCGCAGGCTCGA
CGGCTTATACTTTGGGAACGACATCTTGGCGAACCAGGGCGCAATTGCGCCTGCGCGATT
CTGAGGCCCTTTGTCTATGCTGACCTTCAGCTTCCCCCGCGTACCTGCCCG
Sequence 7630:found in patent application W099/57144
CCGCGGTGGCGGCCGCCCGGNCAGGTACGCGGGGCTGGGCGCGGGGAACTGAAAGCCGGA
AGGGGCAAGACGGGCTCAGTTCGTCATGGGGCTGTTTGGAAAGACCCAGGAGAAGCCGCC
CAAAGAACTGGTCAATGAGTGGTCATTGAAGGTAAGAAAAGGAAATGAGAGTTGTTGACA
GGCAAATAAGGGATATCCANGAAAAAAAAAAAAAAAAAAANAGTACCTT
Sequence 7631:found in patent application W099/53040
CGAATTGGAGCTCCACCCGCGGTGGCGGCCCGCCCGGGCAGGTACTTTTTGCTTTAGCAG
ATAGATAGGGCATCCAATACAACTGAAACAACCTGATAACAAATTAATTTTATTTTTCAA
TAAAAAGGAATGCTCTGGTTTTTTAACTGGCTCCTTGAGGAAGCCAGAAGATGGCATCTG
CTCTTTAAACAGGCTTCTCTTATCTGATTTGAGACACAAATCCACCAAGATTCTTTCTTA
CAGTGGAAGTAGGCACCACCCCTCAGATCACGAGGCCCCTAGGAAGAAGCCTGTGGAAAT
CAGCCTGTGATGTGTGGTGGTGGGCCAGCACCTAGAANACACATGGTGGTTGTGCACATA
AATCCCTTCAGCGGACTCAGTGGAAGGATGAGAATTCTGAAAGTTCATGCACATTTTATT
GAGTAGGTAATATAAAAATGCTTTTTCTTTTTCATTTGTTACAAGTGCATGCTTTGANTG
CCCACCATNTTCAGAAGTCAAAATTACAAAAGG
Sequence 7632:found in patent application W099/53040
CCGCGGTGGCGGCCGCCCGGGCAGGTACGCGGGCGGGGATGGGAGCCACGCCTGAACTAG
AGTTCAGGCTGGATACATGTGCTCACCTGCTGCTCTTGTCTTCCTAAGAGACAGAGAGTG
GGGCAGATGGAGGAGAAGAAAGTGAGGAATGAGTAGCATAGCATTCTGCCAAAAGGGCCC
CAGATTCTTAATTTAGCAAACTAAGAAGCCCAATTAAAAAGCATTGTGGCTAAAGTCTAA
CGCTCCTCTCTTGGTCAGATAACAAAAGCCCTCCCTGTTGGATCTTTTGAAATAAAACGT
GCAAGTTATCCAGGCTCGTAACCTGCATGCTGCCACCTTGAATCCCAAGGAGTATCTGCA
CCTGNAATANCTCTCCACCCCTCTCTGCCTCCTTACTTTCTGTGCAANATNACTTCCTGG
GTTAACTTCCTTCTTTCCATCCACCCACCCACTGAAATCTCTTTCCAAACATTTTTCCAT
TTTCCCACAAATNGNCTTTGATTAACTNTCCTCTCTCCATGCCTNCAAANCTCCAAATTT
TTGGGGAAAGCTGTACCTTCANCCNCTCTAAAACTAATGNATCCCCCCGNCTNCAAGAAT
TCNATATCAAACTTATCAATACCNTCACCTCAAAGGGGGGCCCANTACCCAACTTTTN

TABLE 5-1

| | | Gen Bank Accession |
|---|---|---|
| Order | Clone | Number |
| 1 | 28106 | R40780 |
| 2 | 30148 | R41329 |
| 3 | 37408 | R49695 |
| 4 | 37598 | R51080 |
| 5 | 38554 | R49731 |
| 6 | 40104 | R54594 |
| 7 | 41850 | R52786 |
| 8 | 42747 | R61847 |
| 9 | 46365 | H09774 |
| 10 | 46477 | H09966 |
| 11 | 46694 | H10192 |
| 12 | 47400 | H10413 |
| 13 | 47652 | H11433 |
| 14 | 50080 | H17934 |
| 15 | 50486 | H17618 |
| 16 | 50935 | H18626 |
| 17 | 51378 | H24011 |
| 18 | 51799 | H23524 |
| 19 | 67237 | T52700 |
| 20 | 69002 | T54298 |
| 21 | 69935 | T48692 |
| 22 | 81413 | T60161 |
| 23 | 111812 | T91225 |
| 24 | 120528 | T95320 |
| 25 | 124447 | R01094 |
| 26 | 127524 | R08871 |
| 27 | 133864 | R28660 |
| 28 | 136605 | R35051 |
| 29 | 149288 | R82644 |
| 30 | 151055 | H02231 |
| 31 | 156962 | R74321 |
| 32 | 196435 | R91517 |
| 33 | 197651 | R94504 |
| 34 | 200354 | R96914 |
| 35 | 204483 | H58234 |
| 36 | 205497 | H57857 |
| 37 | 210697 | H66840 |
| 38 | 249070 | H80063 |
| 39 | 258300 | N30680 |
| 40 | 258649 | N32226 |
| 41 | 259950 | N32587 |
| 42 | 266343 | N26556 |
| 43 | 277173 | N34316 |
| 44 | 278729 | N62936 |
| 45 | 280375 | N47113 |
| 46 | 293745 | N65950 |
| 47 | 321834 | W37112 |
| 48 | 344126 | W73753 |
| 49 | 344190 | W69741 |
| 50 | 344721 | W74673 |
| 51 | 346308 | W74133 |
| 52 | 396350 | AA758375 |
| 53 | 399101 | AA733027 |
| 54 | 413148 | AA707871 |
| 55 | 415473 | W80447 |
| 58 | 415610 | W80730 |
| 57 | 415619 | W80741 |
| 58 | 417393 | W88942 |
| 59 | 418150 | W90164 |
| 60 | 418206 | W90529 |
| 61 | 418279 | W90323 |
| 62 | 428560 | AA005401 |
| 63 | 431408 | AA706839 |
| 64 | 432479 | AA699494 |
| 65 | 449428 | AA777883 |
| 66 | 451596 | AA707080 |
| 67 | 451911 | AA706969 |
| 68 | 455275 | AA677575 |
| 69 | 490484 | AA101632 |
| 70 | 501431 | AA115300 |
| 71 | 503741 | AA131466 |
| 72 | 595318 | AA164301 |
| 73 | 625875 | AA186804 |
| 74 | 645565 | AA204830 |
| 75 | 687297 | AA235116 |
| 76 | 712950 | AA282273 |
| 77 | 729953 | AA412049 |
| 78 | 731218 | AA416724 |
| 79 | 742049 | AA401457 |
| 80 | 742569 | AA400074 |
| 81 | 753745 | AA410491 |
| 82 | 755304 | AA436327 |
| 83 | 767289 | AA418418 |
| 84 | 767823 | AA418728 |
| 85 | 768217 | AA424905 |
| 86 | 782233 | AA431721 |
| 87 | 782503 | AA431773 |
| 88 | 785663 | AA449085 |
| 89 | 786525 | AA452113 |
| 90 | 786602 | AA478470 |
| 91 | 788180 | AA453310 |
| 92 | 795499 | AA454215 |
| 93 | 810873 | AA459197 |
| 94 | 810987 | AA485357 |
| 95 | 815183 | AA481152 |
| 96 | 815556 | AA456821 |
| 97 | 824723 | AA488986 |
| 98 | 839081 | AA487608 |
| 99 | 840878 | AA482324 |
| 100 | 841141 | AA487031 |
| 101 | 845345 | AA773478 |
| 102 | 884606 | AA630006 |
| 103 | 897924 | AA598808 |
| 104 | 950470 | AA599102 |
| 105 | 1030855 | AA621761 |
| 106 | 1049033 | AA778675 |
| 107 | 1049185 | AA620697 |
| 108 | 1049230 | AA620715 |
| 109 | 1292432 | AA718910 |
| 110 | 1323448 | AA873604 |
| 111 | 1412481 | AA845156 |
| 112 | 1416782 | AA894557 |
| 113 | 1420370 | AA857035 |
| 114 | 1468220 | AA884897 |
| 115 | 1471841 | AA873355 |
| 116 | 1472753 | AA872402 |

TABLE 5-2

| | | Gen Bank Accession |
|---|---|---|
| Order | Clone | Number |
| 1 | 629896 | AA219045 |
| 2 | 897567 | AA489611 |
| 3 | 856650 | AA669314 |
| 4 | 897781 | AA598517 |
| 5 | 882510 | AA676460 |
| 6 | 586685 | AA130579 |
| 7 | 769890 | AA430382 |
| 8 | 525926 | AA074511 |
| 9 | 714414 | AA293215 |
| 10 | 812251 | AA455056 |
| 11 | 192569 | H41489 |
| 12 | 814989 | AA465723 |
| 13 | 781089 | AA430032 |
| 14 | 757265 | AA426113 |
| 15 | 359661 | AA011096 |
| 16 | 768562 | AA429297 |
| 17 | 811062 | AA485441 |
| 18 | 878676 | AA775355 |
| 19 | 740941 | AA478298 |
| 20 | 684655 | AA251770 |
| 21 | 842863 | AA489261 |
| 22 | 878798 | AA670408 |
| 23 | 281010 | N47691 |
| 24 | 135221 | R32952 |
| 25 | 491751 | AA150500 |
| 26 | 770879 | AA434403 |
| 27 | 290841 | N71982 |
| 28 | 291341 | N72263 |
| 29 | 281003 | N50880 |
| 30 | 785368 | AA476576 |
| 31 | 45582 | H08120 |
| 32 | 739511 | AA478066 |
| 33 | 770043 | AA427570 |
| 34 | 624627 | AA187351 |
| 35 | 416833 | W86653 |
| 36 | 359982 | AA063521 |
| 37 | 669375 | AA253464 |
| 38 | 744047 | AA629262 |
| 39 | 451907 | AA706968 |
| 40 | 757244 | AA426025 |
| 41 | 453107 | AA700904 |
| 42 | 755578 | AA419177 |
| 43 | 502518 | AA156802 |
| 44 | 773286 | AA425299 |
| 45 | 843321 | AA489569 |
| 46 | 278687 | N62924 |
| 47 | 853006 | AA668230 |
| 48 | 897529 | AA497001 |
| 49 | 757165 | AA443950 |
| 50 | 230971 | R96155 |
| 51 | 796624 | AA460530 |
| 52 | 38740 | R51273 |
| 53 | 868757 | AA775325 |
| 54 | 825372 | AA504507 |
| 55 | 746245 | AA417761 |
| 56 | 292399 | N68399 |
| 57 | 626716 | AA191548 |
| 58 | 489106 | AA056538 |
| 59 | 22074 | T66180 |
| 60 | 85800 | T72067 |
| 61 | 705064 | AA279990 |
| 62 | 277740 | N49589 |
| 63 | 35300 | R43798 |
| 64 | 788256 | AA454098 |
| 65 | 746007 | AA482007 |
| 66 | 455263 | AA677572 |
| 67 | 277112 | N39611 |
| 68 | 259591 | N32768 |
| 69 | 22773 | R38613 |
| 70 | 269997 | N24910 |
| 71 | 451918 | AA706964 |
| 72 | 345626 | W72051 |
| 73 | 51041 | H18633 |
| 74 | 32299 | R42685 |
| 75 | 812266 | AA455062 |
| 76 | 41391 | R56123 |
| 77 | 878253 | AA775791 |
| 78 | 83920 | T64312 |
| 79 | 740604 | AA479795 |
| 80 | 770066 | AA430545 |
| 81 | 196189 | R92281 |
| 82 | 46367 | H09959 |
| 83 | 784772 | AA478542 |
| 84 | 246430 | N53031 |
| 85 | 310493 | W31074 |
| 86 | 753162 | AA400457 |
| 87 | 459941 | AA779380 |
| 88 | 200814 | R98851 |
| 89 | 415102 | W95001 |
| 90 | 489677 | AA099568 |
| 91 | 193892 | H51765 |
| 92 | 359781 | AA011320 |
| 93 | 812266 | AA455062 |
| 94 | 277414 | N34436 |
| 95 | 49284 | H15533 |
| 96 | 66317 | T66816 |
| 97 | 239611 | H79534 |
| 98 | 502151 | AA129777 |
| 99 | 773330 | AA425450 |
| 100 | 266819 | N24113 |
| 101 | 29063 | R40970 |
| 102 | 785293 | AA476543 |
| 103 | 824933 | AA489040 |
| 104 | 345616 | W72431 |
| 105 | 144816 | R76281 |
| 106 | 291985 | N73101 |
| 107 | 256907 | N30096 |
| 108 | 487793 | AA043501 |
| 109 | 840776 | AA486082 |
| 110 | 199945 | R97066 |
| 111 | 232946 | H75599 |
| 112 | 197525 | H52001 |
| 113 | 753467 | AA406551 |
| 114 | 771303 | AA443638 |
| 115 | 50879 | H18424 |
| 116 | 341680 | W60414 |
| 117 | 461727 | AA682293 |
| 118 | 345793 | W70128 |
| 119 | 486591 | AA042990 |
| 120 | 345849 | W70343 |
| 121 | 452374 | AA700876 |
| 122 | 241699 | H91641 |
| 123 | 262060 | H99075 |

TABLE 6

| ACC. NO. | GI NUMBER |
|---|---|
| A06805 | 490047 |
| A06846 | 490055 |
| A06919 | 490059 |
| A06925 | 490060 |
| A16794 | 512417 |
| A17362 | 512432 |
| A17546 | 490012 |
| A18585 | 512456 |
| A18657 | 512461 |
| A21185 | 579591 |
| A23013 | 825583 |
| AA001116 | 1437185 |
| AA001199 | 1437284 |
| AA001219 | 1437294 |
| AA001222 | 1437297 |
| AA001234 | 1437357 |
| AA001299 | 1437383 |
| AA001316 | 1437400 |
| AA001319 | 1437403 |
| AA001376 | 1436881 |
| AA001403 | 1436888 |
| AA001414 | 1436899 |
| AA001431 | 1437116 |
| AA001434 | 1437119 |
| AA001444 | 1436975 |
| AA001449 | 1436914 |
| AA001460 | 1436925 |
| AA001536 | 1437001 |
| AA001614 | 1445191 |
| AA001673 | 1445230 |
| AA001699 | 1445335 |
| AA001734 | 1445600 |
| AA001745 | 1445539 |
| AA001792 | 1445606 |
| AA001794 | 1445608 |
| AA001805 | 1445619 |
| AA001834 | 1445648 |
| AA001841 | 1445655 |
| AA001870 | 1445514 |
| AA001876 | 1445520 |
| AA001976 | 1445411 |
| AA001999 | 1445492 |
| AA002007 | 1445500 |
| AA002131 | 1445147 |
| AA002140 | 1445326 |
| AA002166 | 1445081 |
| AA002181 | 1445096 |
| AA002190 | 1445115 |
| AA002222 | 1445157 |
| AA004206 | 1448401 |
| AA004255 | 1448610 |
| AA004412 | 1448057 |
| AA004609 | 1448476 |
| AA004626 | 1448473 |
| AA004642 | 1448179 |
| AA004671 | 1448208 |
| AA004681 | 1448218 |
| AA004794 | 1448291 |
| AA004802 | 1448299 |
| AA004824 | 1448331 |
| AA004835 | 1448312 |
| AA004869 | 1447686 |
| AA004944 | 1447731 |
| AA004946 | 1447733 |
| AA004973 | 1448833 |
| AA005035 | 1447753 |
| AA005153 | 1447808 |
| AA005196 | 1448658 |
| AA005228 | 1448690 |
| AA005254 | 1448756 |
| AA005350 | 1447832 |
| AA005358 | 1448391 |
| AA005386 | 1447848 |
| AA005401 | 1447863 |
| AA005403 | 1447865 |
| AA007276 | 1463310 |
| AA007280 | 1463314 |
| AA007370 | 1463374 |
| AA007389 | 1463627 |
| AA007474 | 1463440 |
| AA007509 | 1463545 |
| AA007516 | 1463492 |
| AA007529 | 1463505 |
| AA007619 | 1463605 |
| AA007687 | 1463679 |
| AA009527 | 1470814 |
| AA009531 | 1470818 |
| AA009609 | 1470750 |
| AA009611 | 1470820 |
| AA009615 | 1470824 |
| AA009641 | 1470856 |
| AA009763 | 1470566 |
| AA009803 | 1470587 |
| AA009818 | 1470798 |
| AA009840 | 1470887 |
| AA009849 | 1470896 |
| AA009878 | 1470905 |
| AAO10019 | 1471263 |
| AA010065 | 1471093 |
| AA010083 | 1471131 |
| AAO10091 | 1471275 |
| AA010128 | 1471156 |
| AA010129 | 1471157 |
| AA010136 | 1471164 |
| PA010167 | 1471343 |
| AA010174 | 1471350 |
| AA010221 | 1471248 |
| AA010223 | 1471250 |
| AA010224 | 1471251 |
| AA010233 | 1471260 |
| AA010236 | 1471282 |
| AA010284 | 1471310 |
| AA010328 | 1471374 |
| AA010383 | 1471419 |
| AA010388 | 1471424 |
| AA010480 | 1471526 |
| AA010607 | 1471633 |
| AA010608 | 1471634 |
| AA010619 | 1471645 |
| AA010682 | 1471709 |
| AA010689 | 1471716 |
| AA010758 | 1471785 |
| AA010828 | 1471854 |
| AA010869 | 1471915 |
| AA010872 | 1471918 |
| AA010932 | 1471959 |
| AA010981 | 1472008 |
| AA010986 | 1472013 |
| AA011000 | 1472027 |
| AAD11070 | 1472098 |
| AA011095 | 1472123 |
| AA011096 | 1472124 |
| AA011176 | 1472203 |
| AA011182 | 1472209 |
| AA011185 | 1472212 |
| AA011203 | 1472315 |
| AA011215 | 1472327 |
| AA011297 | 1472344 |
| AA011320 | 1472367 |
| AA011389 | 1472415 |
| AA011390 | 1472416 |
| AA011445 | 1472542 |
| AA011570 | 1472596 |
| AA011593 | 1472700 |
| AA011597 | 1472704 |
| AA011621 | 1472658 |
| AA011656 | 1472712 |
| AA011685 | 1472731 |
| AA011707 | 1472759 |
| AA012866 | 1473904 |
| AA012867 | 1473905 |
| AA012927 | 1473954 |
| AA013018 | 1474045 |
| AA013090 | 1474126 |
| AA013094 | 1474130 |
| AA013099 | 1474135 |
| AA013245 | 1474272 |
| AA013260 | 1474307 |
| AA013353 | 1474459 |
| AA013355 | 1474461 |
| AA015605 | 1476653 |
| AA015658 | 1476688 |
| AA015793 | 1476960 |
| AA015819 | 1476849 |
| AA015959 | 1477025 |
| AA016116 | 1477147 |
| PA016205 | 1477262 |
| AA016225 | 1477272 |
| AA016274 | 1477367 |
| AA016281 | 1477374 |
| AA016980 | 1479354 |
| AA017096 | 1479260 |
| AA017133 | 1479435 |
| AA017141 | 1479306 |
| AA017175 | 1479340 |
| AA017189 | 1479372 |
| AA017200 | 1479580 |
| AA017213 | 1479376 |
| AA017262 | 1479443 |
| AA017292 | 1479453 |
| AA017379 | 1479590 |
| AA017383 | 1479547 |
| AA017468 | 1479633 |
| AA017469 | 1479634 |
| AA017474 | 1479639 |
| AA017500 | 1479671 |
| AA017512 | 1479729 |
| AA017544 | 1479697 |
| AA017614 | 1479804 |
| AA017706 | 1479895 |
| AA018050 | 1481435 |
| AA018102 | 1481357 |
| AA018202 | 1481521 |
| AA018215 | 1481471 |
| AA018249 | 1481505 |
| AA018338 | 1481805 |
| AA018345 | 1481664 |
| AA018347 | 1481666 |
| AA018449 | 1481704 |
| AA018460 | 1481715 |
| AA018591 | 1481846 |
| AA018675 | 1481940 |
| AA018683 | 1481966 |
| AA018866 | 1482466 |
| AA018886 | 1482277 |
| AA018888 | 1482279 |
| AA018892 | 1482283 |
| AA018979 | 1482371 |
| AA018980 | 1482372 |
| AA019152 | 1482561 |
| AA019316 | 1482727 |
| AA019320 | 1482731 |
| AA019335 | 1482746 |
| AA019377 | 1482806 |
| AA019445 | 1482074 |
| AA019482 | 1482111 |
| AA019498 | 1482327 |
| AA019511 | 1482130 |
| AA019538 | 1482157 |
| AA019781 | 1483100 |
| AA019874 | 1483700 |
| AA020899 | 1484698 |
| AA020941 | 1484676 |
| AA021037 | 1484827 |
| AA021115 | 1484877 |
| AA021247 | 1484963 |
| AA021434 | 1485150 |
| AA021513 | 1485203 |
| AA021544 | 1485428 |
| AA021586 | 1485257 |
| AA021608 | 1485269 |
| AA021628 | 1485289 |
| AA022453 | 1486552 |
| AA022478 | 1486577 |
| AA022496 | 1486587 |
| AA022583 | 1486673 |
| PA022601 | 1486700 |
| AA022636 | 1486838 |
| AA022668 | 1486749 |
| AA022788 | 1486887 |
| AA022937 | 1487036 |
| AA022949 | 1487039 |
| AA022965 | 1487064 |
| AA024398 | 1489406 |
| AA024459 | 1489485 |
| AA024480 | 1489440 |
| AA024532 | 1489341 |
| AA024581 | 1489523 |
| AA024637 | 1489542 |
| AA024796 | 1489719 |
| AA024898 | 1489804 |
| AA024963 | 1489868 |
| AA024967 | 1489872 |
| AA025089 | 1490012 |
| AA025112 | 1490027 |
| AA025142 | 1490056 |
| AA025287 | 1489366 |
| AA025373 | 1490893 |
| AA025434 | 1490916 |
| AA025538 | 1490975 |
| AA025551 | 1490997 |
| AA025601 | 1491020 |
| AA025694 | 1491397 |
| AA025731 | 1491407 |
| AA025834 | 1491180 |
| AA025875 | 1491212 |
| AA025937 | 1491436 |
| AA025968 | 1491277 |
| AA026149 | 1492190 |
| AA026288 | 1492245 |
| AA026343 | 1492787 |
| AA026399 | 1492300 |
| AA026609 | 1492444 |
| AA026630 | 1492465 |
| AA026658 | 1492723 |
| AA026682 | 1492849 |
| AA026715 | 1492496 |
| AA026797 | 1493134 |
| AA026983 | 1493174 |
| AA027037 | 1493227 |
| AA027090 | 1493299 |
| AA027126 | 1493335 |
| AA027147 | 1492816 |
| AA027862 | 1493949 |
| AA027868 | 1493955 |
| AA027882 | 1493979 |
| AA027912 | 1493999 |
| AA028008 | 1494086 |
| AA028103 | 1494190 |
| AA028145 | 1494214 |
| AA028171 | 1494296 |
| AA028887 | 1496309 |
| AA028987 | 1496389 |
| AA029039 | 1496584 |
| AA029070 | 1496472 |
| AA029143 | 1496545 |
| AA029157 | 1496559 |
| AA029276 | 1496887 |
| AA029295 | 1496699 |
| AA029403 | 1497030 |
| AA029415 | 1496958 |
| AA029428 | 1496841 |
| AA029490 | 1496957 |
| AA029566 | 1497034 |
| AA029567 | 1497035 |
| AA029578 | 1496982 |
| AA029584 | 1496988 |
| AA029673 | 1497076 |
| AA029676 | 1497079 |
| AA029722 | 1497135 |
| AA029835 | 1496080 |
| AA029881 | 1496108 |
| AA029889 | 1496145 |
| AA029947 | 1496175 |
| AA029997 | 1496428 |
| AA030004 | 1496296 |
| AA030046 | 1496272 |
| AA031287 | 1501242 |
| AA031293 | 1501248 |
| AA031360 | 1501445 |
| AA031367 | 1501320 |
| AA031483 | 1501417 |
| AA031513 | 1501467 |
| AA031528 | 1501492 |
| AA031624 | 1501585 |
| AA031762 | 1501716 |
| AA031795 | 1501730 |
| AA032084 | 1502056 |
| AA032194 | 1502166 |
| AA032220 | 1502182 |
| AA032221 | 1502183 |
| AA033517 | 1505363 |
| AA033537 | 1505383 |
| AA033564 | 1505457 |
| AA033651 | 1505479 |
| AA033869 | 1505752 |
| AA033901 | 1505719 |
| AA033907 | 1505725 |
| AA033951 | 1505826 |
| AA033973 | 1505782 |
| AA034014 | 1505962 |
| AA034094 | 1505921 |
| AA034115 | 1505972 |
| AA034179 | 1505989 |
| PA034186 | 1505996 |
| AA034226 | 1506054 |
| AA034237 | 1506265 |
| AA034255 | 1506064 |
| AA034322 | 1506193 |
| AA034436 | 1506273 |
| AA034516 | 1506355 |
| AA034912 | 1506875 |
| AA034945 | 1507024 |
| AA034946 | 1506890 |
| AA034979 | 1506923 |
| AA035001 | 1506964 |
| AA035012 | 1507099 |
| AA035090 | 1507288 |
| AA035102 | 1507272 |
| AA035144 | 1507314 |
| AA035171 | 1507484 |
| AA035189 | 1507370 |
| AA035347 | 1506848 |
| AA035384 | 1507108 |
| AA035450 | 1507136 |
| AA035549 | 1507207 |
| AA035558 | 1507216 |
| AA035559 | 1507217 |
| AA035580 | 1507238 |
| AA035604 | 1507551 |
| AA035667 | 1507620 |
| AA035730 | 1507567 |
| AA035742 | 1507570 |
| AA035745 | 1507573 |
| AA035773 | 1507601 |
| AA035790 | 1507636 |
| AA036798 | 1509891 |
| AA036823 | 1509861 |
| AA036974 | 1510031 |
| AA037024 | 1510099 |
| AA037126 | 1512234 |
| AA037138 | 1512246 |
| AA037143 | 1512251 |
| AA037172 | 1512332 |
| AA037190 | 1512299 |
| AA037210 | 1512389 |
| AA037216 | 1512395 |
| AA037217 | 1512342 |
| AA037425 | 1512542 |
| AA037450 | 1512752 |
| AA037483 | 1512582 |
| AA037554 | 1512654 |
| AA037574 | 1512692 |
| AA037620 | 1512756 |
| AA037683 | 1512781 |
| AA037828 | 1512946 |
| AA037857 | 1512993 |
| AA037877 | 1513013 |
| AA037886 | 1513022 |
| AA039274 | 1515552 |
| AA039354 | 1515650 |
| AA039381 | 1515658 |
| AA039504 | 1515800 |
| AA039506 | 1515802 |
| AA039546 | 1515824 |
| AA039713 | 1515992 |
| AA039734 | 1516236 |
| AA039750 | 1516029 |
| AA039807 | 1516296 |
| AA039812 | 1516301 |
| AA039851 | 1516129 |
| PA039967 | 1516280 |
| AA039998 | 1516275 |
| AA040037 | 1516380 |
| AA040127 | 1516405 |
| AA040135 | 1516413 |
| AA040140 | 1516418 |
| AA040180 | 1516596 |
| AA040209 | 1516485 |
| AA040265 | 1516670 |
| AA040332 | 1516663 |
| AA040405 | 1516766 |
| AA040424 | 1516702 |
| AA040437 | 1516715 |
| AA040439 | 1516717 |
| AA040499 | 1516786 |
| AA040617 | 1517030 |
| AA040620 | 1517033 |
| AA040624 | 1517103 |
| AA040625 | 1517104 |
| AA040673 | 1516951 |
| AA040694 | 1517008 |
| AA040710 | 1516988 |
| AA040810 | 1517088 |
| AA040820 | 1517098 |
| AA040861 | 1517157 |
| AA041197 | 1517612 |
| AA041205 | 1517439 |
| AA041239 | 1517473 |
| AA041250 | 1517484 |
| AA041467 | 1517765 |
| AA041494 | 1517728 |
| AA041499 | 1517733 |
| AA042812 | 1522467 |
| AA042813 | 1522468 |
| AA042858 | 1522374 |
| AA042928 | 1522610 |
| AA042990 | 1522505 |
| AA043109 | 1520983 |
| AA043133 | 1521126 |
| AA043137 | 1520991 |
| AA043141 | 1520995 |
| AA043159 | 1521013 |
| AA043228 | 1521083 |
| AA043347 | 1521421 |
| AA043415 | 1521290 |
| AA043431 | 1521354 |
| AA043501 | 1521425 |
| AA043552 | 1521413 |
| AA043594 | 1521460 |
| AA043692 | 1521548 |
| AA043802 | 1521715 |
| AA043865 | 1522012 |
| AA043891 | 1521749 |
| AA043951 | 1521876 |
| AA044004 | 1521862 |
| AA044118 | 1521976 |
| AA044123 | 1521981 |
| AA044187 | 1522044 |
| AA044209 | 1522066 |
| AA044239 | 1522115 |
| AA044291 | 1522148 |
| AA044416 | 1522291 |
| AA044565 | 1522690 |
| AA044570 | 1522895 |
| AA044586 | 1522977 |
| AA044591 | 1522982 |
| AA044599 | 1522823 |
| AA044652 | 1522855 |
| AA044659 | 1523039 |
| AA044662 | 1523042 |
| AA044691 | 1522912 |
| AA044697 | 1522965 |
| AA044750 | 1522953 |
| AA044786 | 1523054 |
| AA044791 | 1522994 |
| AA044896 | 1523100 |
| AA044903 | 1523107 |
| AA044906 | 1523110 |
| AA044941 | 1523144 |
| AA044960 | 1523163 |
| AA045074 | 1523555 |
| AA045115 | 1523317 |
| AA045147 | 1523487 |
| AA045175 | 1523377 |
| AA045185 | 1523387 |
| AA045240 | 1523444 |
| AA045300 | 1523502 |
| AA045340 | 1523542 |
| AA045342 | 1523544 |
| AA045367 | 1523699 |
| AA045381 | 1523582 |
| AA045388 | 1523637 |
| AA045410 | 1523612 |
| AA045436 | 1523674 |
| AA045508 | 1523744 |
| AA045524 | 1523760 |
| AA045574 | 1525319 |
| AA045584 | 1525329 |
| AA045606 | 1525351 |
| AA045665 | 1525596 |
| AA045673 | 1525416 |
| AA045698 | 1525800 |
| AA045699 | 1525801 |
| AA045709 | 1525667 |
| AA045741 | 1525635 |
| AA045753 | 1525647 |
| AA045792 | 1525686 |
| AA045803 | 1525697 |
| PA045870 | 1525809 |
| AA045879 | 1525774 |
| AA045992 | 1526095 |
| AA046040 | 1525934 |
| AA046083 | 1525976 |
| AA046321 | 1526214 |
| AA046430 | 1526341 |
| AA046483 | 1524476 |
| AA046489 | 1524482 |
| AA046523 | 1524626 |
| AA046577 | 1524493 |
| AA046581 | 1524497 |
| AA046705 | 1524602 |
| AA046763 | 1524661 |
| AA046808 | 1524725 |
| AA046836 | 1524735 |
| AA046848 | 1524747 |
| AA046883 | 1524818 |
| AA046973 | 1524871 |
| AA046981 | 1524879 |
| AA047015 | 1524913 |
| AA047021 | 1524937 |
| AA047213 | 1525113 |
| AA047260 | 1525296 |
| AA047304 | 1525220 |
| AA047332 | 1525441 |
| AA047338 | 1525236 |
| AA047340 | 1525238 |
| AA047432 | 1525478 |
| AA047441 | 1525487 |
| AA047482 | 1525657 |
| AA047517 | 1525563 |
| AA047597 | 1527304 |
| AA047618 | 1527272 |
| AA047704 | 1527374 |
| AA047729 | 1527526 |
| AA047752 | 1527422 |
| AA053129 | 1544269 |
| AA053192 | 1544401 |
| AA053239 | 1544449 |
| AA053250 | 1544460 |
| AA053265 | 1544475 |
| AA053399 | 1544036 |
| AA053459 | 1544213 |
| AA053475 | 1544113 |
| AA053510 | 1544337 |
| AA053514 | 1544420 |
| AA053522 | 1544305 |
| AA053557 | 1544341 |
| AA053684 | 1544611 |
| AA053747 | 1544673 |
| AA053757 | 1544683 |
| AA053909 | 1544834 |
| AA053917 | 1544861 |
| AA053932 | 1544876 |
| AA054120 | 1545043 |
| AA054135 | 1545058 |
| AA054439 | 1545575 |
| AA054492 | 1545417 |
| AA054521 | 1545511 |
| AA054585 | 1545654 |
| AA054643 | 1545567 |
| AA054701 | 1545625 |
| AA054778 | 1545714 |
| AA055186 | 1547724 |
| AA055221 | 1547559 |
| AA055285 | 1547623 |
| AA055306 | 1547644 |
| AA055326 | 1547729 |
| AA055475 | 1547880 |
| AA055491 | 1547830 |
| AA055585 | 1547950 |
| AA055650 | 1547989 |
| AA055768 | 1548168 |
| AA055833 | 1548308 |
| AA055835 | 1548237 |
| AA055880 | 1548218 |
| AA055979 | 1548345 |
| AA056001 | 1548387 |
| AA056013 | 1548352 |
| AA056025 | 1548364 |
| AA056034 | 1548373 |
| AA056058 | 1548452 |
| AA056060 | 1548454 |
| AA056232 | 1548569 |
| AA056281 | 1548685 |
| AA056345 | 1548964 |
| AA056358 | 1548698 |
| AA056365 | 1548705 |
| AA056369 | 1548709 |
| AA056395 | 1548735 |
| AA056396 | 1548736 |
| AA056437 | 1548777 |
| AA056462 | 1548802 |
| AA056482 | 1548885 |
| AA056484 | 1548887 |
| AA056528 | 1548868 |
| AA056534 | 1548874 |
| AA056538 | 1548878 |
| AA056603 | 1548943 |
| AA056667 | 1549007 |
| AA056686 | 1549026 |
| AA056744 | 1549119 |
| AA057029 | 1549704 |
| AA057032 | 1549707 |
| AA057063 | 1549950 |
| AA057071 | 1549810 |
| AA057095 | 1549769 |
| AA057156 | 1549615 |
| AA057195 | 1549334 |
| AA057243 | 1550096 |
| AA057270 | 1549908 |
| AA057290 | 1549928 |
| AA057490 | 1550130 |
| AA057541 | 1550246 |
| AA057620 | 1550455 |
| AA057721 | 1550522 |
| AA057861 | 1549571 |
| AA057867 | 1549577 |
| AA058381 | 1551208 |
| AA058398 | 1551413 |
| AA058458 | 1551284 |
| AA058488 | 1551295 |
| AA058568 | 1551375 |
| AA058576 | 1551383 |
| AA058597 | 1551404 |
| AA058605 | 1551430 |
| AA058818 | 1551628 |
| AA058899 | 1551725 |
| AA058902 | 1551728 |
| AA058906 | 1551740 |
| AA059309 | 1552153 |
| AA059355 | 1553179 |
| AA062583 | 1556851 |
| AA062731 | 1557092 |
| AA062804 | 1557164 |
| AA062805 | 1557165 |
| AA062813 | 1557173 |
| AA062859 | 1557360 |
| AA062928 | 1557429 |
| AA062985 | 1557637 |
| AA062994 | 1557655 |
| AA063247 | 1556934 |
| AA063476 | 1557286 |
| AA063501 | 1557311 |
| AA063521 | 1557488 |
| AA063616 | 1557583 |
| AA063629 | 1557596 |
| AA064627 | 1558871 |
| AA064886 | 1559007 |
| AA064947 | 1559211 |
| AA065036 | 1558685 |
| AA065042 | 1558691 |
| AA065100 | 1559022 |
| AA065214 | 1559127 |
| AA065216 | 1559129 |
| AA069078 | 1576438 |
| AA069079 | 1576439 |
| AA069367 | 1576725 |
| AA069407 | 1576838 |
| AA069457 | 1576960 |
| AA069561 | 1576973 |
| AA069692 | 1577112 |
| AA069693 | 1577113 |
| AA069784 | 1577152 |
| AA069801 | 1577169 |
| AA069811 | 1577188 |
| AA069814 | 1577191 |
| AA069845 | 1577205 |
| AA069850 | 1577210 |
| AA069880 | 1577258 |
| AA070008 | 1577368 |
| AA070031 | 1577593 |
| AA070086 | 1577446 |
| AA070226 | 1577585 |
| AA070250 | 1577669 |
| AA070277 | 1577637 |
| AA070331 | 1577690 |
| AA070335 | 1577694 |
| AA070349 | 1577708 |
| AA070393 | 1577754 |
| AA070435 | 1577813 |
| AA070437 | 1577815 |
| AA070495 | 1577855 |
| AA070624 | 1577984 |
| AA070911 | 1578270 |
| AA070997 | 1578418 |
| AA071030 | 1578390 |
| AA071084 | 1578444 |
| AA071089 | 1578449 |
| AA071165 | 1578526 |
| AA071167 | 1578528 |
| AA071199 | 1578577 |
| AA071372 | 1578726 |
| AA071439 | 1578802 |
| AA071503 | 1578874 |
| AA071511 | 1578882 |
| AA074049 | 1614100 |
| AA074067 | 1613937 |
| AA074086 | 1613956 |
| AA074142 | 1614012 |
| AA074183 | 1614246 |
| AA074200 | 1614112 |
| AA074224 | 1614093 |
| AA074258 | 1614180 |
| AA074445 | 1614313 |
| AA074446 | 1614314 |
| AA074509 | 1614378 |
| AA074511 | 1614398 |
| AA074575 | 1614445 |
| AA074666 | 1614610 |
| AA074677 | 1614604 |
| AA074683 | 1614552 |
| AA074729 | 1614736 |
| AA074818 | 1614687 |
| AA074819 | 1614688 |
| AA074837 | 1614706 |
| AA074869 | 1614765 |
| AA075008 | 1614894 |
| AA075012 | 1614957 |
| AA075133 | 1615137 |
| AA075165 | 1615035 |
| AA075189 | 1615059 |
| AA075257 | 1615127 |
| AA075415 | 1615478 |
| AA075459 | 1615330 |
| AA075460 | 1615331 |
| AA075474 | 1615361 |
| AA075515 | 1615385 |
| AA075530 | 1615400 |
| AA075600 | 1615469 |
| AA075645 | 1615515 |
| AA075781 | 1615653 |
| AA075810 | 1615698 |
| AA075922 | 1615827 |
| AA075981 | 1615850 |
| AA076077 | 1615946 |
| AA076171 | 1616241 |
| AA076260 | 1616129 |
| AA076295 | 1616164 |
| AA076430 | 1616317 |
| AA076504 | 1616373 |
| AA076593 | 1616480 |
| AA076964 | 1836648 |
| AA077283 | 1836757 |
| AA077906 | 1837380 |
| AA078218 | 1837692 |
| AA078872 | 1617825 |
| AA078904 | 1617796 |
| AA079041 | 1617933 |
| AA079188 | 1618080 |
| AA079346 | 1618301 |
| AA079366 | 1618260 |
| AA079524 | 1618416 |
| AA079690 | 1618711 |
| AA079706 | 1618598 |
| AA079755 | 1617661 |
| AA079869 | 1618761 |
| AA080889 | 1623378 |
| AA081003 | 1623007 |
| AA081007 | 1623011 |
| AA081082 | 1623000 |
| AA081169 | 1623285 |
| AA081218 | 1623066 |
| AA081355 | 1623169 |
| AA081426 | 1623343 |
| AA081708 | 1623766 |
| AA081712 | 1623770 |
| AA081751 | 1623810 |
| AA081788 | 1623864 |
| AA081800 | 1623876 |
| AA081812 | 1624072 |
| AA081815 | 1623926 |
| AA081842 | 1623901 |
| AA081973 | 1624039 |
| AA082063 | 1624138 |
| AA082087 | 1624179 |
| AA082209 | 1624285 |
| AA082281 | 1624356 |
| AA082342 | 1624416 |
| AA082437 | 1624556 |
| AA082477 | 1624533 |
| AA082502 | 1624683 |
| AA082572 | 1624692 |
| AA082629 | 1624705 |
| AA082685 | 1624743 |
| AA082754 | 1624812 |
| AA082812 | 1624869 |
| AA082829 | 1624886 |
| AA082839 | 1624914 |
| AA082936 | 1624993 |
| AA082943 | 1625000 |
| AA083008 | 1625064 |
| AA083026 | 1625082 |
| AA083096 | 1625154 |
| AA083148 | 1625207 |
| AA083191 | 1625452 |
| AA083207 | 1625264 |
| AA083288 | 1625349 |
| AA083300 | 1625361 |
| AA083345 | 1625405 |
| AA083370 | 1625430 |
| AA083410 | 1625660 |
| AA083411 | 1625661 |
| AA083472 | 1625558 |
| AA083479 | 1625565 |
| AA083481 | 1625542 |
| AA083482 | 1625543 |
| AA083485 | 1625546 |
| AA083510 | 1625570 |
| AA083514 | 1625574 |
| AA083573 | 1625633 |
| AA083603 | 1625858 |
| AA083654 | 1625713 |
| AA083671 | 1625747 |
| AA083772 | 1625830 |
| AA083794 | 1625852 |
| AA083836 | 1625894 |
| AA083868 | 1625926 |
| AA083941 | 1625998 |
| AA084068 | 1626124 |
| AA084197 | 1626263 |
| AA084221 | 1626287 |
| AA084517 | 1626782 |
| AA084552 | 1626608 |
| AA084560 | 1626616 |
| AA084698 | 1626755 |
| AA084987 | 1627045 |
| AA085283 | 1627410 |
| AA085459 | 1628703 |
| AA085480 | 1628730 |
| AA085483 | 1628756 |
| AA085603 | 1629416 |
| AA085632 | 1629533 |
| AA085748 | 1629231 |
| AA085759 | 1629221 |
| AA085810 | 1629342 |
| AA085824 | 1629337 |
| AA085835 | 1629387 |
| AA086007 | 1629574 |
| AA086087 | 1629637 |
| AA086128 | 1629696 |
| AA086204 | 1629010 |
| AA086326 | 1629014 |
| AA086377 | 1629024 |
| AA086475 | 1629122 |
| AA088187 | 1633745 |
| AA088231 | 1633778 |
| AA088322 | 1633826 |
| AA088344 | 1633856 |
| AA088371 | 1633883 |
| AA088372 | 1633884 |
| AA088517 | 1634082 |
| AA088544 | 1634049 |
| AA088603 | 1634124 |
| AA088606 | 1634145 |
| AA088655 | 1634176 |
| AA088679 | 1634200 |
| AA088701 | 1634222 |
| AA088714 | 1634243 |
| AA088755 | 1634276 |
| AA088758 | 1634279 |
| AA088770 | 1634335 |
| AA088850 | 1634371 |
| AA088886 | 1633650 |
| AA088914 | 1633678 |
| AA089566 | 1636074 |
| AA090023 | 1636515 |
| AA090106 | 1636590 |
| AA090203 | 1636727 |
| AA090548 | 1635188 |
| AA090625 | 1635209 |
| AA090790 | 1635366 |
| AA091245 | 1635829 |
| AA091275 | 1635859 |
| AA091277 | 1635861 |
| AA091445 | 1636900 |
| AA091998 | 1637011 |
| AA092060 | 1637457 |
| AA092224 | 1637213 |
| AA092316 | 1637537 |
| AA092343 | 1637636 |
| AA092532 | 1637369 |
| AA092533 | 1637370 |
| AA092596 | 1637377 |
| AA092811 | 1637944 |
| AA093276 | 1638745 |
| AA093359 | 1638984 |
| AA093935 | 1639528 |
| AA093974 | 1639551 |
| AA094000 | 1639577 |
| AA094331 | 1639916 |
| AA094583 | 1640176 |
| AA094656 | 1640233 |
| AA095002 | 1640587 |
| AA095049 | 1640642 |
| AA095478 | 1641095 |
| AA095753 | 1641338 |
| AA096121 | 1641706 |
| AA096320 | 1641913 |
| AA096453 | 1642038 |
| AA098801 | 1648804 |
| AA098824 | 1644905 |
| AA098867 | 1645051 |
| AA098876 | 1644998 |
| AA098896 | 1645062 |
| AA099023 | 1644903 |
| AA099080 | 1645522 |
| AA099110 | 1644971 |
| AA099141 | 1645028 |
| AA099148 | 1645035 |
| AA099262 | 1645108 |
| AA099369 | 1645287 |
| AA099399 | 1645355 |
| AA099404 | 1645297 |
| AA099424 | 1646266 |
| AA099431 | 1645320 |
| AA099520 | 1645466 |
| AA099523 | 1645469 |
| AA099568 | 1645585 |
| AA099572 | 1645589 |
| AA099582 | 1645595 |
| AA099593 | 1645606 |
| AA099602 | 1645730 |
| AA099631 | 1645715 |
| AA099718 | 1645810 |
| AA099820 | 1645919 |
| AA099855 | 1645937 |
| AA099875 | 1645957 |
| AA099878 | 1645960 |
| AA099896 | 1646042 |
| AA099917 | 1646059 |
| AA099952 | 1646129 |
| AA099976 | 1646109 |
| AA100133 | 1646323 |
| AA100195 | 1646472 |
| AA100296 | 1646587 |
| AA100486 | 1646798 |
| AA100674 | 1647035 |
| AA100696 | 1648657 |
| AA100707 | 1647062 |
| AA100852 | 1647269 |
| AA100876 | 1647311 |
| AA100885 | 1647320 |
| AA100987 | 1647714 |
| AA1O1155 | 1647922 |
| AA101173 | 1647992 |
| AA101208 | 1647861 |
| AA101224 | 1648061 |
| AA101237 | 1647877 |
| AA101270 | 1647951 |
| AA101276 | 1647997 |
| AA101299 | 1648045 |
| AA101476 | 1648304 |
| AA101632 | 1648568 |
| AA101674 | 1648628 |
| AA101742 | 1648734 |
| AA101777 | 1648775 |
| AA101799 | 1648810 |
| AA101833 | 1645172 |
| AA101840 | 1645179 |
| AA101878 | 1645281 |
| AA102068 | 1645927 |
| AA102159 | 1646217 |
| AA102183 | 1646391 |
| AA102274 | 1646755 |
| AA102280 | 1646691 |
| AA102570 | 1647762 |
| AA102591 | 1647800 |
| AA102605 | 1647891 |
| AA102670 | 1648004 |
| AA102698 | 1648215 |
| AA111855 | 1663942 |
| AA111892 | 1663963 |
| AA111969 | 1664039 |
| AA112162 | 1664364 |
| AA112198 | 1664602 |
| AA112308 | 1664577 |
| AA112310 | 1664579 |
| AA112441 | 1664851 |
| AA112515 | 1665064 |
| AA112626 | 1665327 |
| AA112660 | 1665361 |
| AA112710 | 1663810 |
| AA112869 | 1664220 |
| AA112936 | 1664286 |
| AA112979 | 1664450 |
| AA113333 | 1665237 |
| AA113347 | 1665187 |
| AA113403 | 1665305 |
| AA113811 | 1667672 |
| AA113823 | 1667801 |
| AA113952 | 1667829 |
| AA114003 | 1668008 |
| AA114014 | 1667891 |
| AA114061 | 1667997 |
| AA114062 | 1667998 |
| AA114078 | 1668143 |
| AA114106 | 1667982 |
| AA114853 | 1669884 |
| AA3A4854 | 1669885 |
| AA114945 | 1670040 |
| AA114952 | 1670065 |
| AA114967 | 1670182 |
| AA115021 | 1670274 |
| AA115076 | 1670339 |
| AA115215 | 1670044 |
| AA115259 | 1670437 |
| AA115297 | 1670475 |
| AA115300 | 1670497 |
| AA115310 | 1670627 |
| AA115492 | 1670163 |
| AA115512 | 1669972 |
| AA115537 | 1670199 |
| AA115559 | 1670404 |
| AA115588 | 1670232 |
| AA115604 | 1670248 |
| AA115636 | 1670771 |
| AA115742 | 1670755 |
| AA115761 | 1670792 |
| AA115838 | 1670916 |
| AA115933 | 1670950 |
| AA116061 | 1671093 |
| AA120779 | 1678093 |
| AA120781 | 1678095 |
| AA120782 | 1678096 |
| AA120816 | 1678011 |
| AA120818 | 1678013 |
| AA120849 | 1678245 |
| AA120852 | 1678322 |
| AA120953 | 1678358 |
| AA120964 | 1678369 |
| AA121072 | 1678675 |
| AA121127 | 1678688 |
| AA121145 | 1678884 |
| AA121266 | 1678899 |
| AA121387 | 1679010 |
| AA121390 | 1679013 |
| AA121419 | 1679096 |
| AA121428 | 1679105 |
| AA121471 | 1679085 |
| AA121504 | 1679118 |
| AA121615 | 1679230 |
| AA121625 | 1679256 |
| AA121656 | 1679269 |
| AA121732 | 1679346 |
| AA121836 | 1679529 |
| AA121923 | 1677937 |
| AA121941 | 1677954 |
| AA122048 | 1678067 |
| AA122080 | 1678118 |
| AA122129 | 1678250 |
| AA122237 | 1678538 |
| AA122245 | 1678484 |
| AA125809 | 1688020 |
| AA125817 | 1687493 |
| AA125825 | 1685546 |
| AA125961 | 1685627 |
| AA125989 | 1685663 |
| AA126009 | 1685675 |
| AA126025 | 1685691 |
| AA126029 | 1685695 |
| AA126121 | 1685796 |
| AA126128 | 1685819 |
| AA126259 | 1687876 |
| AA126356 | 1686004 |
| AA126373 | 1685985 |
| AA126468 | 1686098 |
| AA126472 | 1686102 |
| AA126621 | 1686196 |
| AA126666 | 1686222 |
| AA126682 | 1687580 |
| AA126708 | 1687588 |
| AA126729 | 1686293 |
| AA126742 | 1686242 |
| AA126750 | 1686250 |
| AA126754 | 1686254 |
| AA126799 | 1686281 |
| AA126809 | 1686309 |
| AA126834 | 1686316 |
| AA126921 | 1687896 |
| AA126951 | 1686403 |
| AA127029 | 1687899 |
| AA127069 | 1687662 |
| AA127100 | 1687677 |
| AA127135 | 1686495 |
| AA127136 | 1686480 |
| AA127185 | 1686545 |
| AA127220 | 1687685 |
| AA127336 | 1686643 |
| AA127369 | 1686658 |
| AA127436 | 1686832 |
| AA127493 | 1686783 |
| AA127552 | 1686824 |
| AA127674 | 1686962 |
| AA127714 | 1687003 |
| AA127861 | 1687141 |
| AA128005 | 1687285 |
| AA128104 | 1687383 |
| AA128114 | 1687998 |
| AA128297 | 1688383 |
| AA128306 | 1688356 |
| AA128324 | 1688392 |
| AA128348 | 1688435 |
| AA128387 | 1689834 |
| AA128391 | 1689838 |
| AA128407 | 1689705 |
| AA128442 | 1689853 |
| AA128498 | 1688495 |
| AA128536 | 1688508 |
| AA128591 | 1688535 |
| AA128617 | 1689602 |
| AA128647 | 1688607 |
| AA128668 | 1688603 |
| AA128739 | 1689610 |
| AA128768 | 1688659 |
| AA128952 | 1688735 |
| AA129003 | 1688786 |
| AA129135 | 1688902 |
| AA129171 | 1688955 |
| AA129217 | 1689086 |
| AA129245 | 1689029 |
| AA129297 | 1689080 |
| AA129501 | 1689366 |
| AA129551 | 1689316 |
| AA129552 | 1689317 |
| AA129574 | 1689339 |
| AA129615 | 1689380 |
| AA129652 | 1690081 |
| AA129677 | 1690088 |
| AA129726 | 1690181 |
| AA129772 | 1690183 |
| AA129777 | 1690188 |
| AA129781 | 1689412 |
| AA129907 | 1689483 |
| AA129909 | 1689667 |
| AA129931 | 1689507 |
| AA129962 | 1689684 |
| AA129964 | 1689521 |
| AA130017 | 1690879 |
| AA130042 | 1691037 |
| AA130162 | 1691165 |
| AA130207 | 1691211 |
| AA130229 | 1691233 |
| AA130262 | 1691539 |
| AA130271 | 1691476 |
| AA130302 | 1691446 |
| AA130349 | 1691492 |
| AA130428 | 1691711 |
| AA130515 | 1691798 |
| AA130529 | 1691812 |
| AA130555 | 1692108 |
| AA130579 | 1692001 |
| AA130615 | 1692238 |
| AA130677 | 1692185 |
| AA130784 | 1692450 |
| AA130797 | 1692295 |
| AA130857 | 1692345 |
| AA130873 | 1692361 |
| AA130890 | 1692397 |
| AA130928 | 1692418 |
| AA130946 | 1692436 |
| AA130955 | 1692462 |
| AA130984 | 1692475 |
| AA131077 | 1692567 |
| AA131198 | 1692690 |
| AA131227 | 1692735 |
| AA131233 | 1692741 |
| AA131239 | 1692766 |
| AA131240 | 1692767 |
| AA131252 | 1692814 |
| AA131284 | 1692774 |
| AA131313 | 1692820 |
| AA131327 | 1692825 |
| AA131406 | 1692893 |
| AA131466 | 1693089 |
| AA131516 | 1693022 |
| AA131526 | 1693077 |
| AA131640 | 1693166 |
| AA131707 | 1693197 |
| AA131749 | 1693275 |
| AA131793 | 1693282 |
| AA131812 | 1693319 |
| AA131828 | 1693381 |
| AA131834 | 1693324 |
| AA131885 | 1693437 |
| AA131909 | 1693407 |
| AA131926 | 1693424 |
| AA131961 | 1693653 |
| AA132219 | 1693710 |
| AA132226 | 1693717 |
| AA132342 | 1693832 |
| AA132508 | 1694199 |
| AA132510 | 1694201 |
| AA132574 | 1694081 |
| AA132596 | 1694085 |
| AA132633 | 1694122 |
| AA132689 | 1694179 |
| AA132690 | 1694180 |
| AA132792 | 1694485 |
| AA132798 | 1694347 |
| AA132802 | 1694351 |
| AA132844 | 1694333 |
| AA132857 | 1694362 |
| AA132862 | 1694555 |
| AA132904 | 1694391 |
| AA132956 | 1694454 |
| AA132992 | 1694561 |
| AA133030 | 1694538 |
| AA133044 | 1694569 |
| AA133068 | 1694593 |
| AA133071 | 1694596 |
| AA133165 | 1689945 |
| AA133166 | 1689946 |
| AA133228 | 1690214 |
| AA133248 | 1690341 |
| AA133266 | 1690234 |
| AA133268 | 1690236 |
| AA133276 | 1690244 |
| AA133307 | 1690347 |
| AA133361 | 1690329 |
| AA133382 | 1690384 |
| AA133452 | 1690474 |
| AA133469 | 1690437 |
| AA133503 | 1690623 |
| AA133554 | 1690524 |
| AA133579 | 1690549 |
| AA133590 | 1690603 |
| AA133652 | 1690638 |
| AA133692 | 1690660 |
| AA133698 | 1690666 |
| AA133721 | 1690707 |
| AA133724 | 1690710 |
| AA133742 | 1690774 |
| AA133757 | 1690725 |
| AA133788 | 1690756 |
| AA133798 | 1690785 |
| AA133896 | 1689546 |
| AA133959 | 1691026 |
| AA133982 | 1691068 |
| AA134016 | 1691084 |
| AA134022 | 1691090 |
| AA134023 | 1691091 |
| AA134031 | 1691099 |
| AA134032 | 1691100 |
| AA134126 | 1691471 |
| AA134203 | 1691620 |
| AA134234 | 1691590 |
| AA134266 | 1691640 |
| AA134267 | 1691641 |
| AA134311 | 1691666 |
| AA134321 | 1691676 |
| AA134377 | 1691871 |
| AA134527 | 1692092 |
| AA134576 | 1695573 |
| AA134701 | 1695360 |
| AA134715 | 1695319 |
| AA134726 | 1695303 |
| AA134770 | 1695436 |
| AA134778 | 1695407 |
| AA134862 | 1695363 |
| AA135001 | 1696102 |
| AA135049 | 1696151 |
| AA135065 | 1696167 |
| AA135104 | 1696231 |
| AA135135 | 1697032 |
| AA135222 | 1696324 |
| AA135254 | 1696373 |
| AA135384 | 1696495 |
| AA135454 | 1696503 |
| AA135456 | 1696505 |
| AA135663 | 1696702 |
| AA135809 | 1696820 |
| AA135812 | 1696823 |
| AA135870 | 1696844 |
| AA135875 | 1696849 |
| AA135886 | 1696860 |
| AA135911 | 1697229 |
| AA136049 | 1697259 |
| AA136060 | 1697270 |
| AA136071 | 1697281 |
| AA136096 | 1697324 |
| AA136125 | 1697335 |
| AA136150 | 1697360 |
| AA136163 | 1697419 |
| AA136215 | 1697527 |
| AA136221 | 1697431 |
| AA136296 | 1697504 |
| AA136351 | 1697559 |
| AA136386 | 1697730 |
| AA136404 | 1697614 |
| AA136437 | 1697647 |
| AA136618 | 1697881 |
| AA136664 | 1697892 |
| AA136699 | 1697909 |
| AA136710 | 1697920 |
| AA136794 | 1698003 |
| AA136808 | 1698017 |
| AA136830 | 1698247 |
| AA136874 | 1698084 |
| AA136944 | 1698154 |
| AA137072 | 1698289 |
| AA137078 | 1698295 |
| AA137144 | 1698379 |
| AA137165 | 1696943 |
| AA137170 | 1697210 |
| AA142865 | 1712289 |
| AA142869 | 1712293 |
| AA142913 | 1712300 |
| AA142922 | 1712428 |
| AA142923 | 1712429 |
| AA143012 | 1712389 |
| AA143021 | 1712398 |
| AA143034 | 1712411 |
| AA143170 | 1712540 |
| AA143242 | 1712613 |
| AA143252 | 1712623 |
| AA143286 | 1712657 |
| AA143438 | 1712808 |
| AA143467 | 1712855 |
| AA143523 | 1712894 |
| AA143548 | 1713117 |
| AA143578 | 1712950 |
| AA143579 | 1712951 |
| AA143604 | 1712992 |
| AA143634 | 1713004 |
| AA146582 | 1716005 |
| AA146589 | 1716073 |
| AA146616 | 1716014 |
| AA146701 | 1716091 |
| AA146826 | 1716208 |
| AA146900 | 1716290 |
| AA146916 | 1716336 |
| AA146941 | 1716313 |
| AA146981 | 1716476 |
| AA147100 | 1716491 |
| AA147214 | 1716587 |
| AA147341 | 1716713 |
| AA147403 | 1716774 |
| AA147499 | 1716870 |
| AA147507 | 1716878 |
| AA147594 | 1716983 |
| AA147608 | 1717043 |
| AA147659 | 1717048 |
| AA147812 | 1717384 |
| AA147826 | 1717198 |
| AA147837 | 1717209 |
| AA147871 | 1717261 |
| AA147898 | 1717271 |
| AA147902 | 1717275 |
| AA147911 | 1717284 |
| AA147980 | 1717354 |
| AA147986 | 1717360 |
| AA148002 | 1717376 |
| AA148019 | 1717419 |
| AA148034 | 1717409 |
| AA148087 | 1717646 |
| AA148206 | 1717613 |
| AA148215 | 1717721 |
| AA148219 | 1717725 |
| AA148250 | 1717632 |
| AA148262 | 1717644 |
| AA148268 | 1717666 |
| AA148269 | 1717667 |
| AA148505 | 1721549 |
| AA148569 | 1721593 |
| AA148729 | 1719240 |
| AA148735 | 1719246 |
| AA148780 | 1721831 |
| AA148822 | 1721659 |
| AA148831 | 1721668 |
| AA148862 | 1719158 |
| AA149080 | 1719370 |
| AA149096 | 1719549 |
| AA149219 | 1719654 |
| AA149337 | 1719089 |
| AA149415 | 1719931 |
| AA149421 | 1719937 |
| AA149490 | 1720326 |
| AA149492 | 1720328 |
| AA149543 | 1720344 |
| AA149551 | 1720352 |
| AA149579 | 1720380 |
| AA149624 | 1720425 |
| AA149637 | 1720438 |
| AA149852 | 1720932 |
| AA149936 | 1721088 |
| AA149979 | 1721132 |
| AA150001 | 1721154 |
| AA150033 | 1721319 |
| AA150156 | 1721377 |
| AA150220 | 1721731 |
| AA150267 | 1721788 |
| AA150309 | 1721839 |
| AA150320 | 1721888 |
| AA150361 | 1721873 |
| AA150369 | 1721900 |
| AA150383 | 1721914 |
| AA150401 | 1721977 |
| AA150421 | 1721934 |
| AA150422 | 1721935 |
| AA150435 | 1721948 |
| AA150459 | 1721990 |
| AA150480 | 1721994 |
| AA150500 | 1722014 |
| AA150502 | 1722016 |
| AA150507 | 1722021 |
| AA150619 | 1722197 |
| AA150650 | 1722162 |
| AA150672 | 1722203 |
| AA150777 | 1722288 |
| AA150780 | 1722338 |
| AA150823 | 1722353 |
| AA150839 | 1722414 |
| AA150860 | 1722371 |
| AA150875 | 1722386 |
| AA150891 | 1722421 |
| AA150918 | 1722429 |
| AA150928 | 1722439 |
| AA151002 | 1722513 |
| AA151012 | 1722523 |
| AA1S1O18 | 1722529 |
| AA151163 | 1719283 |
| AA151350 | 1719895 |
| AA151401 | 1719737 |
| AA151486 | 1719991 |
| AA151506 | 1720046 |
| AA151596 | 1720083 |
| AA151659 | 1720214 |
| AA151796 | 1720491 |
| AA151830 | 1720525 |
| AA151917 | 1720790 |
| AA151979 | 1721096 |
| AA151988 | 1720962 |
| AA152009 | 1720847 |
| AA152073 | 1720985 |
| AA152169 | 1721221 |
| AA152178 | 1721230 |
| AA152264 | 1721601 |
| AA152266 | 1721603 |
| AA152276 | 1721679 |
| AA152337 | 1719171 |
| AA152347 | 1719259 |
| AA152408 | 1718618 |
| AA152440 | 1719097 |
| AA155695 | 1727311 |
| AA155787 | 1727404 |
| AA155828 | 1727446 |
| AA155856 | 1727474 |
| AA155865 | 1727500 |
| AA155999 | 1727666 |
| AA156022 | 1727647 |
| AA156030 | 1727655 |
| AA156040 | 1727820 |
| AA156054 | 1727679 |
| AA156092 | 1727708 |
| AA156112 | 1727728 |
| AA156202 | 1727836 |
| AA156215 | 1727895 |
| AA156239 | 1727857 |
| AA156244 | 1727862 |
| AA156342 | 1727976 |
| AA156369 | 1727985 |
| AA156433 | 1728058 |
| AA156599 | 1728344 |
| AA156690 | 1728304 |
| AA156743 | 1728570 |
| AA156782 | 1728397 |
| AA156790 | 1728405 |
| AA156795 | 1728410 |
| AA156802 | 1728435 |
| AA156862 | 1728477 |
| AA156873 | 1728488 |
| AA156879 | 1728512 |
| AA156886 | 1728700 |
| AA156926 | 1728541 |
| AA158940 | 1728555 |
| AA156971 | 1728777 |
| AA156987 | 1728602 |
| AA157008 | 1728652 |
| AA157129 | 1728737 |
| AA157150 | 1728758 |
| AA157163 | 1728787 |
| AA157261 | 1728869 |
| AA157267 | 1728875 |
| AA157466 | 1729073 |
| AA157632 | 1729240 |
| AA157652 | 1729293 |
| AA157723 | 1729348 |
| AA157733 | 1729358 |
| AA157741 | 1729366 |
| AA157787 | 1732598 |
| AA157813 | 1732642 |
| AA457816 | 1732645 |
| AA157875 | 1732686 |
| AA158035 | 1732828 |
| AA158098 | 1732917 |
| AA158162 | 1732956 |
| AA158211 | 1733022 |
| AA158274 | 1733138 |
| AA158346 | 1733157 |
| AA158507 | 1733318 |
| AA158735 | 1733529 |
| AA158922 | 1733733 |
| AA159044 | 1733847 |
| AA159068 | 1733862 |
| AA159179 | 1733972 |
| AA159181 | 1733974 |
| AA159272 | 1734074 |
| AA159497 | 1735040 |
| AA159577 | 1735128 |
| AA159578 | 1735129 |
| AA159613 | 1741841 |
| AA159647 | 1741830 |
| AA159656 | 1734689 |
| AA159714 | 1734360 |
| AA159748 | 1741796 |
| AA159771 | 1741801 |
| AA159774 | 1741804 |
| AA159792 | 1734632 |
| AA159847 | 1735079 |
| AA159849 | 1735081 |
| AA159910 | 1734637 |
| AA160002 | 1734493 |
| AA160053 | 1734518 |
| AA160080 | 1734579 |
| AA160149 | 1734597 |
| AA160380 | 1735174 |
| AA160606 | 1736052 |
| AA160630 | 1736007 |
| AA160670 | 1736055 |
| AA160677 | 1736062 |
| AA160695 | 1736261 |
| AA160999 | 1735706 |
| AA161003 | 1735290 |
| AA161097 | 1735333 |
| AA161188 | 1735442 |
| AA161217 | 1735462 |
| AA161248 | 1735493 |
| AA161283 | 1735519 |
| AA161466 | 1735905 |
| AA164301 | 1741248 |
| AA164369 | 1741316 |
| AA164405 | 1740715 |
| AA164473 | 1740650 |
| AA164607 | 1740775 |
| AA164677 | 1740838 |
| AA164703 | 1741076 |
| AA164782 | 1740943 |
| AA164836 | 1741059 |
| AA164977 | 1740341 |
| AA164983 | 1740409 |
| AA165027 | 1740273 |
| AA165082 | 1740310 |
| AA165090 | 1740318 |
| AA165117 | 1740372 |
| AA165148 | 1740421 |
| AA165152 | 1740425 |
| AA165164 | 1740392 |
| AA165165 | 1740393 |
| AA165197 | 1740633 |
| AA165205 | 1740497 |
| AA165243 | 1740471 |
| AA165290 | 1740518 |
| AA165313 | 1740541 |
| AA165339 | 1741488 |
| AA165402 | 1741435 |
| AA165512 | 1741529 |
| AA165562 | 1741586 |
| AA165593 | 1741617 |
| AA165632 | 1741665 |
| AA165639 | 1741672 |
| AA165678 | 1741729 |
| AA166675 | 1745130 |
| AA166703 | 1745193 |
| AA166707 | 1745346 |
| AA166853 | 1745071 |
| AA166907 | 1745417 |
| AA166925 | 1745301 |
| AA166952 | 1745328 |
| AA167011 | 1745386 |
| AA167044 | 1745437 |
| AA167084 | 1745460 |
| AA167120 | 1745549 |
| AA167150 | 1745526 |
| AA167252 | 1745646 |
| AA167269 | 1745706 |
| AA167338 | 1745732 |
| AA167366 | 1745743 |
| AA167399 | 1745846 |
| AA167500 | 1745885 |
| AA167674 | 1744833 |
| AA167677 | 1744836 |
| AA167696 | 1744855 |
| AA167719 | 1744931 |
| AA467728 | 1744878 |
| AA167766 | 1744916 |
| AA167804 | 1744955 |
| AA167826 | 1744977 |
| AA167829 | 1744980 |
| AA169121 | 1748241 |
| AA169154 | 1747829 |
| AA169173 | 1747749 |
| AA169192 | 1747768 |
| AA169276 | 1748289 |
| AA169374 | 1748314 |
| AA169379 | 1748319 |
| AA169475 | 1747883 |
| AA169493 | 1748207 |
| AA169507 | 1747913 |
| AA169520 | 1747926 |
| AA169606 | 1747994 |
| AA469638 | 1748220 |
| AA169645 | 1748227 |
| AA169736 | 1748362 |
| AA169794 | 1748129 |
| AA171426 | 1750753 |
| AA171454 | 1750512 |
| AA171459 | 1750517 |
| AA171510 | 1750569 |
| AA171516 | 1750575 |
| AA171587 | 1750656 |
| AA171605 | 1750674 |
| AA171618 | 1750694 |
| AA171679 | 1750755 |
| AA171706 | 1750791 |
| AA171784 | 1750842 |
| AA171807 | 1750865 |
| AA171830 | 1750906 |
| AA171872 | 1750930 |
| AA171883 | 1750941 |
| AA171899 | 1750975 |
| AA172043 | 1751119 |
| AA172056 | 1751150 |
| AA172103 | 1751161 |
| AA172156 | 1751215 |
| AA172188 | 1751265 |
| AA172195 | 1751272 |
| AA172239 | 1751298 |
| AA172266 | 1751334 |
| AA172372 | 1751420 |
| AA173098 | 1753340 |
| AA173109 | 1753445 |
| AA173184 | 1754399 |
| AA173189 | 1754404 |
| AA173331 | 1753658 |
| AA173348 | 1753479 |
| AA173363 | 1753494 |
| AA173391 | 1753732 |
| AA173459 | 1753625 |
| AA173460 | 1753626 |
| AA173522 | 1753672 |
| AA173559 | 1753691 |
| AA173598 | 1753748 |
| AA173721 | 1753855 |
| AA173926 | 1754058 |
| AA173948 | 1754098 |
| AA173952 | 1754128 |
| AA173990 | 1754121 |
| AA173997 | 1754290 |
| AA173998 | 1754291 |
| AA174144 | 1754286 |
| AA176178 | 1757300 |
| AA176331 | 1757489 |
| AA176607 | 1757795 |
| AA176747 | 1757904 |
| AA176784 | 1757933 |
| AA176833 | 1757965 |
| AA176847 | 1757979 |
| AA176867 | 1758071 |
| AA176979 | 1758127 |
| AA177050 | 1758198 |
| AA177054 | 1758202 |
| AA177062 | 1758210 |
| AA177127 | 1758285 |
| AA178907 | 1760276 |
| AA179098 | 1760450 |
| AA179105 | 1760457 |
| AA179161 | 1760512 |
| AA179187 | 1760556 |
| AA179315 | 1760667 |
| AA179362 | 1760713 |
| AA179409 | 1760961 |
| AA179442 | 1760794 |
| AA179462 | 1760830 |
| AA179487 | 1760847 |
| AA179567 | 1760935 |
| AA179664 | 1761878 |
| AA179691 | 1761924 |
| AA180137 | 1761403 |
| AA180154 | 1761446 |
| AA180163 | 1761627 |
| AA180204 | 1761470 |
| AA180270 | 1761537 |
| AA180398 | 1761662 |
| AA180453 | 1761717 |
| AA180817 | 1764292 |
| AA180845 | 1764320 |
| AA180914 | 1764390 |
| AA180941 | 1764433 |
| AA181017 | 1764219 |
| AA181102 | 1764568 |
| AA181111 | 1764577 |
| AA181113 | 1764596 |
| AA181179 | 1764664 |
| AA181392 | 1764876 |
| AA181526 | 1765010 |
| AA181580 | 1765046 |
| AA181618 | 1765085 |
| AA181646 | 1765113 |
| AA181723 | 1765190 |
| AA181767 | 1765234 |
| AA181855 | 1765322 |
| AA181995 | 1765496 |
| AA182409 | 1766232 |
| AA182457 | 1766155 |
| AA182502 | 1766201 |
| AA182513 | 1766212 |
| AA182617 | 1766506 |
| AA182680 | 1766519 |
| AA182804 | 1766530 |
| AA182839 | 1766556 |
| AA182848 | 1766582 |
| AA182948 | 1766631 |
| AA186335 | 1774563 |
| AA186346 | 1774631 |
| AA186364 | 1774465 |
| AA186399 | 1774699 |
| AA186406 | 1774706 |
| AA186432 | 1774532 |
| AA186477 | 1774694 |
| AA186538 | 1774764 |
| AA186586 | 1774685 |
| AA186608 | 1774783 |
| AA186613 | 1774771 |
| AA186733 | 1774850 |
| AA186755 | 1774855 |
| AA186804 | 1774922 |
| AA186895 | 1774997 |
| AA187207 | 1773433 |
| AA187287 | 1773496 |
| AA187288 | 1773497 |
| AA187351 | 1773561 |
| AA187383 | 1773576 |
| AA187395 | 1773621 |
| AA187426 | 1773745 |
| AA187576 | 1773769 |
| AA187595 | 1773788 |
| AA187604 | 1773797 |
| AA187629 | 1773883 |
| AA187659 | 1773851 |
| AA187679 | 1773871 |
| AA187710 | 1774043 |
| AA187762 | 1774031 |
| AA187789 | 1773983 |
| AA187809 | 1774003 |
| AA187817 | 1774011 |
| AA187928 | 1774120 |
| AA187982 | 1774429 |
| AA187998 | 1774189 |
| AA188045 | 1774295 |
| AA188051 | 1774244 |
| AA188052 | 1774245 |
| AA188065 | 1774258 |
| AA188101 | 1774354 |
| AA188140 | 1774332 |
| AA188169 | 1774370 |
| AA188228 | 1774430 |
| AA188312 | 1775338 |
| AA188315 | 1775341 |
| AA188366 | 1775400 |
| AA188396 | 1775430 |
| AA188591 | 1775616 |
| AA188633 | 1775863 |
| AA188710 | 1775797 |
| AA188771 | 1776003 |
| AA188780 | 1775871 |
| AA188854 | 1775943 |
| AA188867 | 1775894 |
| AA188875 | 1775902 |
| AA188918 | 1776005 |
| AA188999 | 1776024 |
| AA189106 | 1776158 |
| AA189113 | 1776165 |
| AA189150 | 1776194 |
| AA190313 | 1779023 |
| AA190408 | 1779239 |
| AA190529 | 1779522 |
| AA190561 | 1779536 |
| AA190615 | 1779781 |
| AA190634 | 1779747 |
| AA190661 | 1779073 |
| AA190674 | 1779018 |
| AA190676 | 1779020 |
| AA190747 | 1779133 |
| AA190779 | 1779361 |
| AA190789 | 1779174 |
| AA190825 | 1779210 |
| AA190843 | 1779416 |
| AA190850 | 1779496 |
| AA190852 | 1779498 |
| AA190873 | 1779393 |
| AA190890 | 1779428 |
| AA190914 | 1779570 |
| AA190997 | 1779702 |
| AA190998 | 1779703 |
| AA191045 | 1779655 |
| AA191092 | 1779684 |
| AA191119 | 1779813 |
| AA191134 | 1779828 |
| AA191211 | 1779905 |
| AA191245 | 1779952 |
| AA191424 | 1780103 |
| AA191461 | 1780168 |
| AA191476 | 1780138 |
| AA191495 | 1780175 |
| AA191512 | 1780237 |
| AA191518 | 1780181 |
| AA191536 | 1780199 |
| AA191548 | 1780211 |
| AA191552 | 1780215 |
| AA191661 | 1780332 |
| AA191719 | 1780399 |
| AA191721 | 1780401 |
| AA192527 | 1781749 |
| AA192597 | 1781819 |
| AA192604 | 1781826 |
| AA192759 | 1782156 |
| AA192765 | 1782162 |
| AA192784 | 1782198 |
| AA193086 | 1782574 |
| AA193247 | 1782657 |
| AA193254 | 1782699 |
| AA193297 | 1782689 |
| AA193346 | 1782936 |
| AA193411 | 1783011 |
| AA193455 | 1782846 |
| AA193661 | 1783072 |
| AA194062 | 1783808 |
| AA194162 | 1783853 |
| AA194189 | 1783943 |
| AA194374 | 1784080 |
| AA194429 | 1784190 |
| AA194473 | 1784169 |
| AA194517 | 1784213 |
| AA194535 | 1784231 |
| AA194577 | 1784338 |
| AA194646 | 1784563 |
| AA194754 | 1784444 |
| AA194860 | 1784572 |
| AA194988 | 1784690 |
| AA195010 | 1784712 |
| AA195015 | 1784717 |
| AA195044 | 1784987 |
| AA195063 | 1784827 |
| AA195145 | 1784917 |
| AA195151 | 1784840 |
| AA195259 | 1784959 |
| AA195377 | 1785090 |
| AA195470 | 1785183 |
| AA195525 | 1785228 |
| AA195560 | 1785264 |
| AA195579 | 1785283 |
| AA195601 | 1785305 |
| AA195617 | 1783794 |
| AA195651 | 1785340 |
| AA195668 | 1785346 |
| AA195734 | 1785433 |
| AA195751 | 1785450 |
| AA195831 | 1791422 |
| AA195947 | 1791538 |
| AA196001 | 1791625 |
| AA196287 | 1791869 |
| AA196357 | 1791932 |
| AA196392 | 1791951 |
| AA196465 | 1792039 |
| AA196486 | 1792044 |
| AA196558 | 1792141 |
| AA196597 | 1792188 |
| AA196978 | 1792569 |
| AA196979 | 1792570 |
| AA197003 | 1792594 |
| AA197334 | 1792925 |
| AA197344 | 1791370 |
| AA199658 | 1795365 |
| AA199717 | 1795425 |
| AA199802 | 1795511 |
| AA199881 | 1795588 |
| AA203110 | 1798853 |
| AA203119 | 1798871 |
| AA203172 | 1798882 |
| AA203182 | 1798892 |
| AA203206 | 1798916 |
| AA203237 | 1799127 |
| AA203252 | 1798970 |
| AA203285 | 1799011 |
| AA203462 | 1799327 |
| AA203492 | 1799465 |
| AA203537 | 1799248 |
| AA203592 | 1799301 |
| AA203637 | 1799356 |
| AA204650 | 1802633 |
| AA204701 | 1802552 |
| AA204703 | 1802554 |
| AA204781 | 1802789 |
| AA204830 | 1802846 |
| AA204847 | 1802855 |
| AA204854 | 1802862 |
| AA204856 | 1802881 |
| AA205033 | 1803023 |
| AA205320 | 1803310 |
| AA205412 | 1803420 |
| AA205413 | 1803541 |
| AA205496 | 1803613 |
| AA205546 | 1803554 |
| AA205647 | 1803639 |
| AA205803 | 1801192 |
| AA205857 | 1801228 |
| AA205858 | 1801229 |
| AA205870 | 1801441 |
| AA205928 | 1801316 |
| AA205949 | 1801381 |
| AA205996 | 1801367 |
| AA206004 | 1801393 |
| AA206115 | 1801486 |
| AA206137 | 1801508 |
| AA206147 | 1801535 |
| AA206194 | 1801591 |
| AA206214 | 1801602 |
| AA206267 | 1801637 |
| AA206311 | 1801681 |
| AA206356 | 1801806 |
| AA206540 | 1801921 |
| AA206546 | 1801927 |
| AA206581 | 1801961 |
| AA206647 | 1802017 |
| AA206675 | 1802045 |
| AA206721 | 1802091 |
| AA206769 | 1802346 |
| AA206865 | 1802235 |
| AA206914 | 1802491 |
| AA206991 | 1801246 |
| AA207105 | 1802456 |
| AA207266 | 1802759 |
| AA209369 | 1807295 |
| AA209498 | 1807467 |
| AA210741 | 1809404 |
| AA210760 | 1809414 |
| AA210833 | 1809523 |
| AA210840 | 1809475 |
| AA210898 | 1809544 |
| AA211448 | 1810093 |
| AA211483 | 1810137 |
| AA211498 | 1810152 |
| AA211565 | 1810219 |
| AA211653 | 1810299 |
| AA211799 | 1810435 |
| AA213548 | 1812185 |
| AA213619 | 1812256 |
| AA213667 | 1812286 |
| AA213669 | 1812288 |
| AA214031 | 1812669 |
| AA214648 | 1813302 |
| AA214668 | 1813322 |
| AA214688 | 1814476 |
| AA214710 | 1814498 |
| AA215284 | 1815047 |
| AA215332 | 1815086 |
| AA215538 | 1815292 |
| AA215628 | 1815391 |
| AA215777 | 1815531 |
| AA215793 | 1815574 |
| AA215800 | 1815553 |
| AA215927 | 1815866 |
| AA216133 | 1816072 |
| AA216211 | 1816150 |
| AA216391 | 1816352 |
| AA216409 | 1816361 |
| AA216433 | 1816397 |
| AA216669 | 1817395 |
| AA216690 | 1817372 |
| AA218536 | 1832711 |
| AA218606 | 1832681 |
| AA218693 | 1832785 |
| AA218788 | 1832880 |
| AA218858 | 1832986 |
| AA218936 | 1833002 |
| AA219045 | 1833137 |
| AA219060 | 1833179 |
| AA219116 | 1833343 |
| AA219143 | 1833208 |
| AA219224 | 1833298 |
| AA219264 | 1833329 |
| AA219315 | 1833381 |
| AA219458 | 1833514 |
| AA219582 | 1833641 |
| AA219705 | 1833788 |
| AA220219 | 1838248 |
| AA220921 | 1839676 |
| AA220966 | 1839709 |
| AA220970 | 1839713 |
| AA223148 | 1843707 |
| AA223249 | 1843790 |
| AA223306 | 1843830 |
| AA223320 | 1843941 |
| AA223425 | 1844136 |
| AA223433 | 1844000 |
| AA223564 | 1844148 |
| AA223774 | 1844333 |
| AA223956 | 1844551 |
| AA224124 | 1844683 |
| AA224132 | 1844691 |
| AA224230 | 1844755 |
| AA224269 | 1844828 |
| AA224487 | 1845130 |
| AA224528 | 1845053 |
| AA224548 | 1845073 |
| AA224751 | 1846097 |
| AA224784 | 1846208 |
| AA224876 | 1846242 |
| AA224953 | 1846262 |
| AA224985 | 1846276 |
| AA224994 | 1846285 |
| AA225002 | 1846311 |
| AA225025 | 1846389 |
| AA225114 | 1846488 |
| AA225115 | 1846489 |
| AA225154 | 1846520 |
| AA225294 | 1846612 |
| AA225438 | 1846765 |
| AA225458 | 1846803 |
| AA225488 | 1846815 |
| AA225515 | 1846823 |
| AA225517 | 1846325 |
| AA225843 | 1847151 |
| AA225857 | 1847165 |
| AA225865 | 1847173 |
| AA226012 | 1847339 |
| AA226101 | 1847438 |
| AA226137 | 1847500 |
| AA226171 | 1847487 |
| AA226234 | 1847561 |
| AA226266 | 1847583 |
| AA226275 | 1847592 |
| AA226321 | 1847697 |
| AA226340 | 1847647 |
| AA226359 | 1847666 |
| AA226430 | 1847736 |
| AA226611 | 1847919 |
| AA226709 | 1848034 |
| AA226735 | 1848306 |
| AA226898 | 1848479 |
| AA227000 | 1848571 |
| AA227059 | 1848622 |
| AA227105 | 1848660 |
| AA227326 | 1848962 |
| AA227506 | 1849051 |
| AA227549 | 1849102 |
| AA227579 | 1849195 |
| AA227647 | 1849209 |
| AA227695 | 1849249 |
| AA227789 | 1849343 |
| AA227830 | 1849393 |
| AA227837 | 1849408 |
| AA227846 | 1849417 |
| AA227873 | 1849426 |
| AA228012 | 1849563 |
| AA228130 | 1849692 |
| AA228273 | 1849853 |
| AA228282 | 1849843 |
| AA228431 | 1849982 |
| AA228440 | 1850000 |
| AA228822 | 1851679 |
| AA228836 | 1851723 |
| AA228845 | 1851654 |
| AA228940 | 1851759 |
| AA228953 | 1851772 |
| AA229057 | 1852041 |
| AA229145 | 1850997 |
| AA229183 | 1851044 |
| AA229199 | 1851031 |
| AA229225 | 1851057 |
| AA229424 | 1851328 |
| AA229495 | 1851569 |
| AA229513 | 1851502 |
| AA229607 | 1851604 |
| AA229611 | 1851608 |
| AA229713 | 1851876 |
| AA229745 | 1851889 |
| AA229872 | 1852224 |
| AA229993 | 1852287 |
| AA230067 | 1852360 |
| AA230169 | 1852500 |
| AA232147 | 1855502 |
| AA232186 | 1855558 |
| AA232249 | 1855602 |
| AA232374 | 1855160 |
| AA232645 | 1855647 |
| AA232686 | 1855688 |
| AA232691 | 1855703 |
| AA232700 | 1855900 |
| AA232734 | 1855727 |
| AA232738 | 1855731 |
| AA232843 | 1855973 |
| AA233021 | 1856014 |
| AA233053 | 1856065 |
| AA233070 | 1856186 |
| AA233073 | 1856189 |
| AA233088 | 1856082 |
| AA233109 | 1856103 |
| AA233131 | 1856207 |
| AA233233 | 1856236 |
| AA233409 | 1856422 |
| AA233428 | 1856485 |
| AA233549 | 1856602 |
| AA233687 | 1856680 |
| AA233689 | 1856699 |
| AA233733 | 1856764 |
| AA233791 | 1856793 |
| AA233809 | 1856811 |
| AA233867 | 1856879 |
| AA233868 | 1856880 |
| AA233943 | 1855138 |
| AA234050 | 1858191 |
| AA234073 | 1859091 |
| AA234092 | 1858897 |
| AA234130 | 1858067 |
| AA234135 | 1858347 |
| AA234149 | 1858244 |
| AA234300 | 1858593 |
| AA234318 | 1858720 |
| AA234464 | 1858983 |
| AA234533 | 1859152 |
| AA234578 | 1859069 |
| AA234646 | 1859139 |
| AA234667 | 1859178 |
| AA234698 | 1859191 |
| AA234818 | 1859348 |
| AA234990 | 1858122 |
| AA235116 | 1859553 |
| AA235224 | 1859662 |
| AA235370 | 1859808 |
| AA235581 | 1860019 |
| AA235802 | 1860239 |
| AA235838 | 1860276 |
| AA235914 | 1860353 |
| AA236057 | 1860765 |
| AA236320 | 1858509 |
| AA236323 | 1858512 |
| AA236371 | 1858514 |
| AA236418 | 1858543 |
| AA236561 | 1858833 |
| AA236667 | 1860705 |
| AA236720 | 1860740 |
| AA236774 | 1860839 |
| AA236865 | 1860895 |
| AA236904 | 1860934 |
| AA236986 | 1861014 |
| AA242864 | 1873858 |
| AA242891 | 1873684 |
| AA242951 | 1873746 |
| AA243065 | 1873878 |
| AA243142 | 1874137 |
| AA243143 | 1874138 |
| AA243230 | 1874023 |
| AA243361 | 1874218 |
| AA243573 | 1874578 |
| AA243696 | 1874488 |
| AA243809 | 1874620 |
| AA243835 | 1874646 |
| AA243980 | 1874809 |
| AA244003 | 1874726 |
| AA244018 | 1874741 |
| AA244052 | 1875041 |
| AA244066 | 1874818 |
| AA244071 | 1874775 |
| AA244099 | 1874803 |
| AA244116 | 1874848 |
| AA244158 | 1874861 |
| AA244184 | 1874905 |
| AA244207 | 1874909 |
| AA247232 | 1878689 |
| AA247638 | 1879471 |
| AA247736 | 1879873 |
| AA247816 | 1880025 |
| AA248319 | 1879036 |
| AA248427 | 1879264 |
| AA249118 | 1879747 |
| AA249154 | 1879783 |
| AA249195 | 1879852 |
| AA250748 | 1885903 |
| AA250846 | 1885808 |
| AA250854 | 1885834 |
| AA250903 | 1885863 |
| AA250947 | 1885973 |
| AA251114 | 1886076 |
| AA251178 | 1886143 |
| AA251182 | 1886147 |
| AA251201 | 1886165 |
| AA251242 | 1886205 |
| AA251262 | 1886225 |
| AA251303 | 1886484 |
| AA251321 | 1886284 |
| AA251386 | 1886413 |
| AA251578 | 1886542 |
| AA251627 | 1886591 |
| AA251644 | 1886608 |
| AA251678 | 1886641 |
| AA251770 | 1886778 |
| AA251784 | 1886746 |
| AA251826 | 1886853 |
| AA252355 | 1887318 |
| AA252436 | 1887407 |
| AA252468 | 1887449 |
| AA252849 | 1887848 |
| AA252943 | 1882696 |
| AA253248 | 1882957 |
| AA253254 | 1882963 |
| AA253269 | 1882978 |
| AA253339 | 1885439 |
| AA253384 | 1885689 |
| AA253442 | 1885634 |
| AA253444 | 1885636 |
| AA253453 | 1885754 |
| AA253464 | 1885639 |
| AA255502 | 1892406 |
| AA255526 | 1892285 |
| AA255529 | 1892288 |
| AA255551 | 1892310 |
| AA255576 | 1892548 |
| AA255577 | 1892549 |
| AA255695 | 1892633 |
| AA255717 | 1892655 |
| AA255761 | 1891319 |
| AA255954 | 1891521 |
| AA255974 | 1891650 |
| AA256117 | 1891675 |
| AA256157 | 1891696 |
| AA256231 | 1891770 |
| AA256248 | 1891787 |
| AA256290 | 1891827 |
| AA256297 | 1891834 |
| AA256330 | 1891867 |
| AA256401 | 1891958 |
| AA256403 | 1891960 |
| AA256420 | 1892179 |
| AA256422 | 1892181 |
| AA256458 | 1891996 |
| AA256507 | 1892114 |
| AA256591 | 1892130 |
| AA256724 | 1892622 |
| Ak256757 | 1892521 |
| AA257014 | 1891143 |
| AA257955 | 1894405 |
| AA257973 | 1894468 |
| AA258003 | 1894435 |
| AA258079 | 1894511 |
| AA258089 | 1894521 |
| AA258357 | 1893489 |
| AA258513 | 1893638 |
| AA258750 | 1893873 |
| AA258904 | 1894047 |
| AA258916 | 1894261 |
| AA259025 | 1894296 |
| AA259062 | 1894542 |
| AA259115 | 1894550 |
| AA259166 | 1894601 |
| AA259189 | 1894716 |
| AA261819 | 1897716 |
| AA261873 | 1897725 |
| AA261889 | 1897932 |
| AA261968 | 1897949 |
| AA261990 | 1897971 |
| AA262080 | 1898204 |
| AA262123 | 1898249 |
| AA262211 | 1898482 |
| AA262226 | 1898497 |
| AA262296 | 1898568 |
| AA262345 | 1898766 |
| AA262494 | 1897873 |
| AA262508 | 1898003 |
| AA262513 | 1897875 |
| AA262537 | 1897899 |
| AA262559 | 1898011 |
| AA262573 | 1898136 |
| AA262588 | 1898023 |
| AA262683 | 1898095 |
| AA262783 | 1898338 |
| AA263002 | 1898824 |
| AA263040 | 1898838 |
| AA263042 | 1898840 |
| AA263145 | 1898951 |
| AA263153 | 1898959 |
| AA263162 | 1898968 |
| AA278195 | 1920153 |
| AA278241 | 1920181 |
| A~278320 | 1920259 |
| AA278357 | 1921665 |
| AA278382 | 1921626 |
| AA278408 | 1919836 |
| AA278445 | 1919782 |
| AA278473 | 1919792 |
| AA278475 | 1919794 |
| AA278520 | 1919858 |
| AA278642 | 1919962 |
| AA278729 | 1920049 |
| AA278749 | 1920096 |
| AA278757 | 1920285 |
| AA278759 | 1920287 |
| AA278785 | 1920106 |
| AA278842 | 1920363 |
| AA278956 | 1920495 |
| AA279054 | 1920519 |
| AA279060 | 1920525 |
| AA279145 | 1920611 |
| AA279172 | 1920655 |
| AA279262 | 1920936 |
| AA279347 | 1920811 |
| AA279409 | 1920938 |
| AA279517 | 1920982 |
| AA279853 | 1921318 |
| AA279949 | 1921414 |
| AA279990 | 1921519 |
| AA280054 | 1921592 |
| AA280080 | 1921554 |
| AA280089 | 1921563 |
| AA280091 | 1921565 |
| AA280221 | 1921759 |
| AA280256 | 1921794 |
| AA280288 | 1922026 |
| AA280381 | 1922038 |
| AA280406 | 1921944 |
| AA280647 | 1924757 |
| AA280694 | 1923375 |
| AA280735 | 1923414 |
| AA280753 | 1923432 |
| AA280848 | 1923546 |
| AA280873 | 1923553 |
| AA280876 | 1923556 |
| AA280917 | 1923597 |
| AA280952 | 1923633 |
| AA281132 | 1923830 |
| AA281307 | 1923988 |
| AA281504 | 1924201 |
| AA281637 | 1924316 |
| AA281653 | 1924556 |
| AA281784 | 1924464 |
| AA281793 | 1924620 |
| AA282023 | 1924855 |
| AA282134 | 1925013 |
| AA282159 | 1925240 |
| AA282215 | 1925131 |
| AA282252 | 1925186 |
| AA282253 | 1925187 |
| AA282272 | 1925386 |
| AA282273 | 1925387 |
| AA282286 | 1925202 |
| AA282382 | 1925298 |
| AA282594 | 1925510 |
| AA282734 | 1925660 |
| AA282837 | 1925797 |
| AA282956 | 1925870 |
| AA282983 | 1925916 |
| AA283007 | 1925931 |
| AA283029 | 1925953 |
| AA283112 | 1926055 |
| AA283169 | 1926103 |
| AA283616 | 1927889 |
| AA283693 | 1927905 |
| AA283704 | 1927916 |
| AA283746 | 1928027 |
| AA283904 | 1928378 |
| AA283949 | 1928247 |
| AA284064 | 1928345 |
| AA284072 | 1928353 |
| AA284108 | 1928585 |
| AA284113 | 1928590 |
| AA284161 | 1928462 |
| AA284172 | 1928665 |
| AA284267 | 1928600 |
| AA284278 | 1928560 |
| AA284280 | 1928562 |
| AA284283 | 1928565 |
| AA284285 | 1928567 |
| AA284291 | 1928573 |
| AA284307 | 1928606 |
| AA284337 | 1928617 |
| AA284410 | 1928690 |
| AA284494 | 1928811 |
| AA284506 | 1928850 |
| AA284555 | 1927484 |
| AA284615 | 1927544 |
| AA284634 | 1927750 |
| AA284642 | 1927553 |
| AA284748 | 1927289 |
| AA284806 | 1927529 |
| AA284835 | 1927376 |
| AA284856 | 1927415 |
| AA284971 | 1927652 |
| AA284997 | 1927885 |
| AA285038 | 1927719 |
| AA285040 | 1927721 |
| AA285043 | 1927724 |
| AA285053 | 1927734 |
| AA285089 | 1927843 |
| AA285290 | 1929600 |
| AA285341 | 2947919 |
| AA286678 | 1933069 |
| AA286708 | 1933704 |
| AA286754 | 1933617 |
| AA286814 | 1933677 |
| AA286841 | 1933866 |
| AA286914 | 1933939 |
| AA286953 | 1933960 |
| AA286965 | 1933972 |
| AA287067 | 1934091 |
| AA287122 | 1934147 |
| AA287199 | 1934250 |
| AA287323 | 1933023 |
| AA287347 | 1933030 |
| AA287375 | 1933057 |
| AA287404 | 1933139 |
| AA287511 | 1933192 |
| AA287561 | 1933243 |
| AA287589 | 1933271 |
| AA287665 | 1933348 |
| AA287720 | 1933411 |
| AA287721 | 1933412 |
| AA287751 | 1933634 |
| AA287936 | 1933759 |
| AA287948 | 1933771 |
| AA287961 | 1933917 |
| AA290591 | 1938853 |
| AA290737 | 1938594 |
| AA290783 | 1938634 |
| AA291041 | 1939019 |
| AA291137 | 1939133 |
| AA291220 | 1939198 |
| AA291233 | 1939211 |
| AA291260 | 1939238 |
| AA291325 | 1939330 |
| AA291363 | 1939341 |
| AA291407 | 1939403 |
| AA291436 | 1939415 |
| AA291556 | 1939730 |
| AA291602 | 1939579 |
| AA291680 | 1939658 |
| AA291742 | 1939721 |
| AA291749 | 1939745 |
| AA291756 | 1939752 |
| AA291764 | 1939760 |
| AA291773 | 1939941 |
| AA291788 | 1939765 |
| AA291972 | 1940028 |
| AA292011 | 1939988 |
| AA292054 | 1940040 |
| AA292131 | 1940109 |
| AA292132 | 1940110 |
| AA292158 | 1940136 |
| AA292179 | 1940174 |
| AA292226 | 1940362 |
| AA292273 | 1940253 |
| AA292281 | 1940261 |
| AA292429 | 1940408 |
| AA292443 | 1940422 |
| AA292583 | 1940577 |
| AA292638 | 1940623 |
| AA292642 | 1940627 |
| AA292655 | 1940711 |
| AA292692 | 1940686 |
| AA292739 | 1941693 |
| AA292786 | 1941608 |
| AA292796 | 1941618 |
| AA292866 | 1941687 |
| AA292871 | 1941852 |
| AA292993 | 1940906 |
| AA292995 | 1940908 |
| AA293027 | 1940923 |
| AA293028 | 1940924 |
| AA293036 | 1940932 |
| AA293047 | 1940943 |
| AA293050 | 1940946 |
| AA293215 | 1941363 |
| AA293629 | 1941280 |
| AA293802 | 1941725 |
| AA293819 | 1941742 |
| AA293855 | 1941833 |
| AA293873 | 1941801 |
| AA293885 | 1941813 |
| AA295182 | 1947516 |
| AA295706 | 1948051 |
| AA295737 | 1948132 |
| AA295762 | 1948097 |
| AA295788 | 1948123 |
| AA295996 | 1948333 |
| AA296074 | 1948410 |
| AA296152 | 1948496 |
| AA296186 | 1948560 |
| AA296237 | 1948581 |
| AA296282 | 1948657 |
| AA296298 | 1948632 |
| AA296326 | 1948701 |
| AA296465 | 1948799 |
| AA296502 | 1948836 |
| AA296534 | 1948940 |
| AA296690 | 1949182 |
| AA296812 | 1949146 |
| AA296846 | 1949180 |
| AA296970 | 1949355 |
| AA297097 | 1949451 |
| AA297215 | 1949549 |
| AA297270 | 1949767 |
| AA297402 | 1949735 |
| AA297432 | 1949927 |
| AA297434 | 1949929 |
| AA297472 | 1949805 |
| AA297506 | 1949860 |
| AA297512 | 1949866 |
| AA297592 | 1950078 |
| AA297647 | 1949981 |
| AA297750 | 1950103 |
| AA297883 | 1950216 |
| AA297941 | 1950295 |
| AA298085 | 1950481 |
| AA298104 | 1950436 |
| AA298199 | 1950530 |
| AA298285 | 1950628 |
| AA298294 | 1950637 |
| AA298464 | 1950807 |
| AA298475 | 1950818 |
| AA298489 | 1950842 |
| AA298490 | 1950843 |
| AA298549 | 1950882 |
| AA298593 | 1950976 |
| AA298596 | 1950979 |
| AA298732 | 1951085 |
| AA298773 | 1951105 |
| AA298786 | 1951118 |
| AA298794 | 1951126 |
| AA298807 | 1951139 |
| AA298967 | 1951299 |
| AA298986 | 1951338 |
| AA299140 | 1951564 |
| AA299209 | 1951550 |
| AA299404 | 1951976 |
| AA299541 | 1951873 |
| AA299594 | 1951925 |
| AA299595 | 1951926 |
| AA299668 | 1952071 |
| AA299830 | 1952394 |
| AA299840 | 1952171 |
| AA299892 | 1952244 |
| AA299961 | 1952291 |
| AA300008 | 1952412 |
| AA300065 | 1952416 |
| AA300262 | 1952595 |
| AA300440 | 1952813 |
| AA300659 | 1953065 |
| AA300728 | 1953296 |
| AA300869 | 1953201 |
| AA301096 | 1953543 |
| AA301205 | 1953538 |
| AA301391 | 1953945 |
| AA301408 | 1953742 |
| AA301513 | 1953847 |
| AA301629 | 1954113 |
| AA301666 | 1954009 |
| AA301753 | 1954096 |
| AA301795 | 1954199 |
| AA301907 | 1954240 |
| AA301983 | 1954316 |
| AA302313 | 1954644 |
| AA302457 | 1954788 |
| AA302480 | 1954811 |
| AA302548 | 1954920 |
| AA302852 | 1955248 |
| AA302919 | 1955270 |
| AA302986 | 1955316 |
| AA303172 | 1955689 |
| AA303184 | 1955537 |
| AA303257 | 1955590 |
| AA303334 | 1955668 |
| AA303594 | 1955986 |
| AA303657 | 1956009 |
| AA303682 | 1956034 |
| AA303696 | 1956038 |
| AA303796 | 1956127 |
| AA303880 | 1956212 |
| AA303924 | 1956409 |
| AA303960 | 1956293 |
| AA304025 | 1956358 |
| AA304651 | 1957302 |
| AA304827 | 1957382 |
| AA304845 | 1957172 |
| AA304846 | 1957173 |
| AA304947 | 1957274 |
| AA304979 | 1957326 |
| AA305030 | 1957356 |
| AA305081 | 1957483 |
| AA305121 | 1957449 |
| AA305143 | 1957492 |
| AA305193 | 1957520 |
| AA305231 | 1957578 |
| AA305315 | 1957683 |
| AA305319 | 1957687 |
| AA305331 | 1957895 |
| AA305333 | 1957897 |
| AA305358 | 1957725 |
| AA305409 | 1957811 |
| AA305494 | 1958072 |
| AA305526 | 1957851 |
| AA305566 | 1957913 |
| AA305599 | 1957924 |
| AA305627 | 1957952 |
| AA305697 | 1958023 |
| AA305774 | 1958102 |
| AA305819 | 1958147 |
| AA305824 | 1958152 |
| AA305895 | 1958225 |
| AA305909 | 1958258 |
| AA305951 | 1958280 |
| AA305969 | 1958298 |
| AA306028 | 1958356 |
| AA306040 | 1958562 |
| AA306129 | 1958457 |
| AA306182 | 1958510 |
| AA306206 | 1958534 |
| AA306223 | 1958551 |
| AA306232 | 1958560 |
| AA306264 | 1958592 |
| AA306322 | 1958670 |
| AA306372 | 1958699 |
| AA306378 | 1958705 |
| AA306402 | 1958750 |
| AA306413 | 1958811 |
| AA306480 | 1958829 |
| AA306540 | 1958869 |
| AA306573 | 1959135 |
| AA306668 | 1959221 |
| AA306772 | 1959100 |
| AA306787 | 1959115 |
| AA306812 | 1959293 |
| AA306876 | 1959204 |
| AA306883 | 1959211 |
| AA306888 | 1959237 |
| AA306902 | 1959382 |
| AA306956 | 1959286 |
| AA306982 | 1959472 |
| AA306983 | 1959384 |
| AA307033 | 1959362 |
| AA307101 | 1959431 |
| AA307103 | 1959433 |
| AA307209 | 1959557 |
| AA307224 | 1959613 |
| AA307247 | 1959575 |
| AA307286 | 1959634 |
| AA307371 | 1959720 |
| AA307475 | 1960023 |
| AA307513 | 1959842 |
| AA307539 | 1959908 |
| AA307579 | 1959948 |
| AA307590 | 1959920 |
| AA307612 | 1959961 |
| AA307669 | 1959998 |
| AA307697 | 1960187 |
| AA307716 | 1960114 |
| AA307720 | 1960048 |
| AA307728 | 1960056 |
| AA307746 | 1960074 |
| AA307789 | 1960339 |
| AA307814 | 1960141 |
| AA307849 | 1960198 |
| AA307857 | 1960206 |
| AA307908 | 1960236 |
| AA307941 | 1960290 |
| AA308051 | 1960430 |
| AA308063 | 1960392 |
| AA308091 | 1960440 |
| AA308207 | 1960536 |
| AA308237 | 1960566 |
| AA308269 | 1960748 |
| AA308273 | 1960672 |
| AA308316 | 1960645 |
| AA308332 | 1960681 |
| AA308486 | 1960835 |
| AA308533 | 1960861 |
| AA308574 | 1960922 |
| AA308578 | 1960926 |
| AA308812 | 1961305 |
| AA308942 | 1961485 |
| AA308978 | 1961326 |
| AA309002 | 1961399 |
| AA309127 | 1961452 |
| AA309445 | 1961986 |
| AA309749 | 1962098 |
| AA309812 | 1962141 |
| AA309832 | 1962325 |
| AA309909 | 1962258 |
| AA310057 | 1962386 |
| AA310142 | 1962470 |
| AA310163 | 1962511 |
| AA310173 | 1962737 |
| AA310181 | 1962579 |
| AA310201 | 1962528 |
| AA310292 | 1962620 |
| AA310441 | 1962769 |
| AA310460 | 1962788 |
| AA310469 | 1962797 |
| AA310561 | 1962887 |
| AA310591 | 1962938 |
| AA310609 | 1962988 |
| AA310637 | 1962964 |
| AA310663 | 1963155 |
| AA310739 | 1963088 |
| AA310807 | 1963135 |
| AA310928 | 1963472 |
| AA310978 | 1963326 |
| AA311028 | 1963355 |
| AA311044 | 1963371 |
| AA311048 | 1963375 |
| AA311125 | 1963483 |
| AA311227 | 1963627 |
| AA311466 | 1963792 |
| AA311505 | 1964050 |
| AA311639 | 1963968 |
| AA311677 | 1964005 |
| AA311772 | 1964099 |
| AA311819 | 1964219 |
| AA311860 | 1964189 |
| AA311896 | 1964546 |
| AA311905 | 1964306 |
| AA311978 | 1964472 |
| AA312025 | 1964354 |
| AA312059 | 1964429 |
| AA312076 | 1964403 |
| AA312078 | 1964405 |
| AA312177 | 1964506 |
| AA312242 | 1964580 |
| AA312406 | 1964734 |
| AA312444 | 1964952 |
| AA312448 | 1964956 |
| AA312495 | 1964824 |
| AA312503 | 1964832 |
| AA312591 | 1964970 |
| AA312605 | 1964944 |
| AA312652 | 1965001 |
| AA312669 | 1965018 |
| AA312689 | 1965038 |
| AA312756 | 1965084 |
| AA312864 | 1965212 |
| AA312962 | 1965352 |
| AA312968 | 1965595 |
| AA313057 | 1965385 |
| AA313164 | 1965492 |
| AA313170 | 1965498 |
| AA313200 | 1965559 |
| AA313208 | 1965669 |
| AA313209 | 1965670 |
| AA313223 | 1965552 |
| AA313244 | 1965573 |
| AA313369 | 1965909 |
| AA313418 | 1965767 |
| AA313468 | 1965797 |
| AA313534 | 1965864 |
| AA313549 | 1965879 |
| AA313647 | 1965977 |
| AA313653 | 1965983 |
| AA313684 | 1966083 |
| AA313692 | 1966021 |
| AA313693 | 1966022 |
| AA313708 | 1966037 |
| AA313778 | 1966107 |
| AA313779 | 1966108 |
| AA313828 | 1966388 |
| AA313837 | 1966237 |
| AA313922 | 1966472 |
| AA313933 | 1966262 |
| AA313979 | 1966326 |
| AA313996 | 1966557 |
| AA314022 | 1966351 |
| AA314028 | 1966357 |
| AA314061 | 1966410 |
| AA314127 | 1966456 |
| AA314146 | 1966495 |
| AA314161 | 1966560 |
| AA314181 | 1966510 |
| AA314188 | 1966517 |
| AA314202 | 1966531 |
| AA314203 | 1966532 |
| AA314241 | 1966642 |
| AA314350 | 1966679 |
| AA314355 | 1966875 |
| AA314391 | 1966740 |
| AA314465 | 1966814 |
| AA314473 | 1966842 |
| AA314599 | 1966927 |
| AA314659 | 1966987 |
| AA314673 | 1967001 |
| AA314748 | 1967077 |
| AA314757 | 1967086 |
| AA314804 | 1967203 |
| AA314810 | 1967209 |
| AA314847 | 1967176 |
| AA314862 | 1967191 |
| AA314872 | 1967221 |
| AA314882 | 1967452 |
| AA314893 | 1967293 |
| AA314907 | 1967236 |
| AA314920 | 1967249 |
| AA314941 | 1967270 |
| AA314961 | 1967379 |
| AA315016 | 1967346 |
| AA315049 | 1967529 |
| AA315069 | 1967408 |
| AA315072 | 1967411 |
| Ak315095 | 1967434 |
| AA315118 | 1967597 |
| AA315172 | 1967501 |
| AA315188 | 1967537 |
| AA315189 | 1967538 |
| AA315215 | 1967614 |
| AA315259 | 1967588 |
| AA315311 | 1967640 |
| AA315321 | 1967650 |
| AA325331 | 1967660 |
| AA315406 | 1967734 |
| AA315416 | 1967775 |
| AA315441 | 1967920 |
| AA315444 | 1967923 |
| AA315460 | 1967789 |
| AA315497 | 1967826 |
| AA315510 | 1967859 |
| AA315555 | 1968084 |
| AA315582 | 1967911 |
| AA315621 | 1968011 |
| AA315689 | 1968107 |
| AA315718 | 1968047 |
| AA315762 | 1968111 |
| AA315780 | 1968119 |
| AA315805 | 1968144 |
| AA315838 | 1968187 |
| AA315896 | 1968224 |
| AA315943 | 1968272 |
| AA315950 | 1968279 |
| AA315968 | 1968297 |
| AA315993 | 1968342 |
| AA316059 | 1968388 |
| AA316110 | 1968439 |
| AA316159 | 1968508 |
| AA316181 | 1968662 |
| AA316199 | 1968528 |
| AA316207 | 1968536 |
| AA316217 | 1968546 |
| AA316322 | 1968671 |
| AA316411 | 1968891 |
| AA316423 | 1968973 |
| AA316627 | 1968955 |
| AA316632 | 1968960 |
| AA316649 | 1969037 |
| AA316721 | 1969070 |
| AA316723 | 1969072 |
| AA316788 | 1969117 |
| AA316962 | 1969310 |
| AA317040 | 1969368 |
| AA317068 | 1969547 |
| AA317088 | 1969426 |
| AA317096 | 1969434 |
| AA317310 | 1969721 |
| AA317455 | 1969803 |
| AA317477 | 1969878 |
| AA317496 | 1969823 |
| AA317497 | 1969824 |
| AA317659 | 1969985 |
| AA317662 | 1969988 |
| AA317855 | 1970181 |
| AA317937 | 1970263 |
| AA317942 | 1970444 |
| AA317952 | 1970300 |
| AA317956 | 1970304 |
| AA317976 | 1970541 |
| AA318009 | 1970377 |
| AA318100 | 1970457 |
| AA318260 | 1970588 |
| AA318272 | 1970600 |
| AA318284 | 1970633 |
| AA318422 | 1970750 |
| AA318544 | 1971106 |
| AA318712 | 1971091 |
| AA318745 | 1971189 |
| AA318969 | 1971371 |
| AA319070 | 1971398 |
| AA319139 | 1971816 |
| AA319538 | 1971886 |
| AA319601 | 1972034 |
| AA319870 | 1972218 |
| AA320004 | 1972351 |
| AA320034 | 1972402 |
| AA320114 | 1972463 |
| AA320603 | 1972952 |
| AA320611 | 1973011 |
| AA320701 | 1973180 |
| AA320723 | 1973051 |
| AA321222 | 1973590 |
| AA321319 | 1973646 |
| AA321703 | 1974040 |
| AA321706 | 1974043 |
| AA322115 | 1974581 |
| AA322246 | 1974573 |
| AA322352 | 1974926 |
| AA322540 | 1974866 |
| AA322728 | 1975054 |
| AA322901 | 1975227 |
| AA322922 | 1975248 |
| AA323143 | 1975544 |
| AA323181 | 1975506 |
| AA323813 | 1976204 |
| AA324251 | 1976494 |
| AA324252 | 1976495 |
| AA324411 | 1976902 |
| AA324478 | 1976744 |
| AA324683 | 1976929 |
| AA324705 | 1976951 |
| AA324826 | 1977091 |
| AA324831 | 1977237 |
| AA325077 | 1977575 |
| AA325108 | 1977363 |
| AA325145 | 1977400 |
| AA325214 | 1977458 |
| AA325220 | 1977464 |
| AA325221 | 1977465 |
| AA325580 | 1977823 |
| AA325594 | 1978084 |
| AA325658 | 1977901 |
| AA325789 | 1978032 |
| AA325826 | 1978069 |
| AA326165 | 1978408 |
| AA326242 | 1978486 |
| AA326251 | 1978517 |
| AA326306 | 1978551 |
| AA326359 | 1978849 |
| AA326752 | 1979007 |
| AA326793 | 1979059 |
| AA326833 | 1979089 |
| AA326895 | 1979141 |
| AA327011 | 1979258 |
| AA327105 | 1979372 |
| AA327228 | 1979534 |
| AA327469 | 1979715 |
| AA327546 | 1979791 |
| AA327611 | 1979876 |
| AA328061 | 1980305 |
| AA328366 | 1980942 |
| AA328446 | 1980711 |
| AA328770 | 1981035 |
| AA329110 | 1981374 |
| AA329242 | 1981486 |
| AA329553 | 1982040 |
| AA329562 | 1981879 |
| AA329658 | 1981900 |
| AA329770 | 1982013 |
| AA329937 | 1982190 |
| AA330090 | 1982332 |
| AA330123 | 1982365 |
| AA330234 | 1982562 |
| AA330784 | 1983026 |
| AA330883 | 1983125 |
| AA331359 | 1983602 |
| AA331859 | 1984101 |
| AA331876 | 1984118 |
| AA332083 | 1984346 |
| AA332139 | 1984392 |
| AA332202 | 1984444 |
| AA332551 | 1984804 |
| AA332556 | 1984809 |
| AA332593 | 1985086 |
| AA332667 | 1984931 |
| AA332716 | 1984959 |
| AA332737 | 1984980 |
| AA333090 | 1985397 |
| AA333300 | 1985574 |
| AA333307 | 1985550 |
| AA333358 | 1985601 |
| AA333526 | 1985779 |
| AA333765 | 1986234 |
| AA334344 | 1986660 |
| AA334403 | 1986667 |
| AA334452 | 1986696 |
| AA334457 | 1986701 |
| AA334511 | 1986795 |
| AA334710 | 1986964 |
| AA334735 | 1986989 |
| AA334754 | 1987018 |
| AA335273 | 1987516 |
| AA335356 | 1987600 |
| AA335563 | 1987826 |
| AA335699 | 1987940 |
| AA336081 | 1988391 |
| AA336197 | 1988435 |
| AA336387 | 1988636 |
| AA336412 | 1988720 |
| AA336586 | 1988824 |
| AA336621 | 1988859 |
| AA336683 | 1988922 |
| AA336740 | 1989131 |
| AA336798 | 1989037 |
| AA337114 | 1989351 |
| AA337836 | 1990075 |
| AA338215 | 1990494 |
| AA338257 | 1990535 |
| AA338836 | 1991074 |
| AA339065 | 1991313 |
| AA339151 | 1991504 |
| AA339735 | 1991972 |
| AA339960 | 1992227 |
| AA340095 | 1992333 |
| AA340608 | 1992866 |
| AA340632 | 1992945 |
| AA340634 | 1992947 |
| AA340635 | 1992948 |
| AA340654 | 1992891 |
| AA340719 | 1993029 |
| AA340927 | 1993166 |
| AA341690 | 1993947 |
| AA341987 | 1994222 |
| AA342231 | 1994467 |
| AA342701 | 1994938 |
| AA342832 | 1995069 |
| AA342969 | 1995205 |
| AA343168 | 1995489 |
| AA343532 | 1995771 |
| AA344067 | 1996336 |
| AA344131 | 1996389 |
| AA344133 | 1996391 |
| AA344376 | 1996635 |
| AA344393 | 1996703 |
| AA344600 | 1996836 |
| AA344625 | 1996882 |
| AA344743 | 1996980 |
| AA345027 | 1997265 |
| AA345139 | 1997447 |
| AA345189 | 1997424 |
| AA345558 | 1997806 |
| AA345624 | 1997892 |
| AA345762 | 1998174 |
| AA345876 | 1998186 |
| AA345904 | 1998142 |
| AA345906 | 1998144 |
| AA345925 | 1998163 |
| AA346253 | 1998490 |
| AA346504 | 1998761 |
| AA346600 | 1998930 |
| AA346844 | 1999101 |
| AA346918 | 1999155 |
| AA347085 | 1999331 |
| AA347236 | 1999472 |
| AA347293 | 1999530 |
| AA347340 | 1999577 |
| AA347390 | 1999626 |
| AA347436 | 1999693 |
| AA347577 | 1999815 |
| AA347889 | 2000124 |
| AA348116 | 2000596 |
| AA348669 | 2000938 |
| AA348734 | 2000971 |
| AA348878 | 2001164 |
| AA349118 | 2001593 |
| AA349192 | 2001668 |
| AA349223 | 2001460 |
| AA349232 | 2001469 |
| AA349464 | 2001702 |
| AA349541 | 2001780 |
| AA349865 | 2002235 |
| AA350206 | 2002525 |
| AA350228 | 2002547 |
| AA350661 | 2002999 |
| AA351201 | 2003520 |
| AA351297 | 2003616 |
| AA351443 | 2003763 |
| AA351504 | 2003896 |
| AA351741 | 2004100 |
| AA351799 | 2004117 |
| AA352062 | 2004402 |
| AA352131 | 2004471 |
| AA352179 | 2004499 |
| AA352755 | 2005084 |
| AA352926 | 2005246 |
| AA352938 | 2005278 |
| AA353655 | 2005975 |
| AA353766 | 2006126 |
| AA354196 | 2006525 |
| AA354245 | 2006585 |
| AA354341 | 2006712 |
| AA354376 | 2006696 |
| AA354391 | 2006895 |
| AA354420 | 2006760 |
| AA354527 | 2006846 |
| AA354730 | 2007049 |
| AA354742 | 2007082 |
| AA354872 | 2007306 |
| AA354915 | 2007473 |
| AA355003 | 2007559 |
| AA355036 | 2007354 |
| AA355119 | 2007436 |
| AA355196 | 2007526 |
| AA355264 | 2007802 |
| AA355308 | 2007638 |
| AA355351 | 2007671 |
| AA355363 | 2007683 |
| AA355451 | 2007812 |
| AA355553 | 2007872 |
| AA355890 | 2008229 |
| AA355956 | 2008274 |
| AA356069 | 2008479 |
| AA356436 | 2008755 |
| AA356682 | 2009000 |
| AA357069 | 2009388 |
| AA357077 | 2009396 |
| AA357574 | 2009894 |
| AA357593 | 2009913 |
| AA357811 | 2010130 |
| AA358169 | 2010487 |
| AA358369 | 2010915 |
| AA358450 | 2010995 |
| AA358790 | 2011108 |
| AA359003 | 2011321 |
| AA359284 | 2011697 |
| AA359418 | 2011757 |
| AA359514 | 2011852 |
| AA359763 | 2012080 |
| AA359832 | 2012183 |
| AA359838 | 2012157 |
| AA360190 | 2012729 |
| AA360310 | 2012628 |
| AA360977 | 2013297 |
| AA361042 | 2013362 |
| AA361225 | 2013543 |
| AA361403 | 2013742 |
| AA361618 | 2013939 |
| AA361640 | 2013961 |
| AA361997 | 2014318 |
| AA362731 | 2015102 |
| AA362778 | 2015169 |
| AA362825 | 2015144 |
| AA362826 | 2015145 |
| AA363019 | 2015357 |
| AA363049 | 2015367 |
| AA363142 | 2015460 |
| AA363481 | 2015801 |
| AA364171 | 2016510 |
| AA364190 | 2016749 |
| AA364261 | 2016600 |
| AA364498 | 2016817 |
| AA364656 | 2016973 |
| AA364667 | 2017006 |
| AA364707 | 2017078 |
| AA364844 | 2017182 |
| AA364882 | 2017199 |
| AA364913 | 2017252 |
| AA365150 | 2017466 |
| AA365229 | 2017545 |
| AA365620 | 2017940 |
| AA365883 | 2018201 |
| AA366269 | 2018588 |
| AA366415 | 2018765 |
| AA366518 | 2018846 |
| AA366865 | 2019235 |
| AA366876 | 2019194 |
| AA366995 | 2019354 |
| AA367471 | 2019789 |
| AA367581 | 2019974 |
| AA367948 | 2020265 |
| AA368520 | 2020859 |
| AA368792 | 2021110 |
| AA368810 | 2021128 |
| AA369521 | 2022146 |
| AA369660 | 2021999 |
| AA369786 | 2022106 |
| AA369804 | 2022124 |
| AA370110 | 2022426 |
| AA370116 | 2022432 |
| AA370122 | 2022438 |
| AA370222 | 2022539 |
| AA370337 | 2022718 |
| AA370352 | 2022901 |
| AA370501 | 2022914 |
| AA370507 | 2022920 |
| AA370548 | 2022868 |
| AA370620 | 2022960 |
| AA370976 | 2023294 |
| AA371017 | 2023335 |
| AA371157 | 2023496 |
| AA371205 | 2023523 |
| AA371265 | 2023659 |
| AA371483 | 2023801 |
| AA371613 | 2023933 |
| AA371626 | 2023946 |
| AA371964 | 2024282 |
| AA372171 | 2024719 |
| AA372911 | 2025251 |
| AA373079 | 2025419 |
| AA373289 | 2025894 |
| AA373314 | 2025633 |
| AA373628 | 2025948 |
| AA373756 | 2026076 |
| AA374020 | 2026340 |
| AA374073 | 2026413 |
| AA374198 | 2026517 |
| AA374222 | 2026561 |
| AA374376 | 2026715 |
| AA374561 | 2026952 |
| AA374718 | 2027263 |
| AA374766 | 2027086 |
| AA374814 | 2027195 |
| AA375135 | 2027455 |
| AA375164 | 2027524 |
| AA375298 | 2027628 |
| AA375312 | 2027642 |
| AA375454 | 2027845 |
| AA375483 | 2027803 |
| AA375531 | 2028085 |
| AA375853 | 2028408 |
| AA375927 | 2028266 |
| AA375988 | 2028306 |
| AA376034 | 2028354 |
| AA376123 | 2028443 |
| AA376277 | 2028648 |
| AA376292 | 2028612 |
| AA376424 | 2028814 |
| AA376668 | 2029214 |
| AA376847 | 2029164 |
| AA376870 | 2029187 |
| AA377218 | 2029535 |
| AA377796 | 2030114 |
| AA378059 | 2030398 |
| AA378226 | 2030606 |
| AA378508 | 2030828 |
| AA378589 | 2030909 |
| AA378933 | 2031252 |
| AA379015 | 2031333 |
| AA379370 | 2031708 |
| AA379448 | 2031766 |
| AA379449 | 2031788 |
| AA380769 | 2033159 |
| AA381480 | 2033891 |
| AA382481 | 2035042 |
| AA383011 | 2035328 |
| AA383100 | 2035481 |
| AA383420 | 2035800 |
| AA384125 | 2036454 |
| AA384272 | 2036591 |
| AA384322 | 2036641 |
| AA384529 | 2036879 |
| AA384756 | 2037169 |
| AA384987 | 2037305 |
| AA385204 | 2037521 |
| AA385388 | 2037706 |
| AA385449 | 2037766 |
| AA385573 | 2037892 |
| AA386056 | 2038414 |
| AA386087 | 2038424 |
| AA386267 | 2038603 |
| AA386326 | 2038728 |
| AA393061 | 2046224 |
| AA393080 | 2046022 |
| AA393259 | 2046359 |
| AA393313 | 2046281 |
| AA393336 | 2046510 |
| AA393408 | 2046429 |
| AA393480 | 2046646 |
| AA393485 | 2046453 |
| AA393650 | 2046620 |
| AA393699 | 2046668 |
| AA393816 | 2046783 |
| AA393870 | 2046875 |
| AA393886 | 2046855 |
| AA393906 | 2047017 |
| AA394176 | 2047163 |
| AA394298 | 2047293 |
| AA394299 | 2047294 |
| AA394302 | 2047297 |
| AA394311 | 2046088 |
| AA397448 | 2050489 |
| AA397452 | 2050493 |
| AA397460 | 2050501 |
| AA397702 | 2050804 |
| AA397802 | 2051010 |
| AA397813 | 2051021 |
| AA397832 | 2050589 |
| AA397953 | 2051098 |
| AA398214 | 2051323 |
| AA398218 | 2051345 |
| AA398247 | 2051356 |
| AA398256 | 2051365 |
| AA398262 | 2051371 |
| AA398267 | 2051376 |
| AA398321 | 2051430 |
| AA398348 | 2051457 |
| AA398355 | 2051464 |
| AA398384 | 2051556 |
| AA398406 | 2051515 |
| AA398463 | 2051573 |
| AA398521 | 2051694 |
| AA398619 | 2051728 |
| AA398674 | 2051787 |
| AA398686 | 2051799 |
| AA398732 | 2051854 |
| AA398757 | 2051916 |
| AA398922 | 2051959 |
| AA398929 | 2051966 |
| AA398949 | 2052886 |
| AA399024 | 2052823 |
| AA399070 | 2052808 |
| AA399074 | 2052829 |
| AA399133 | 2052871 |
| AA399177 | 2052913 |
| AA399198 | 2052934 |
| AA399218 | 2052971 |
| AA399223 | 2053160 |
| AA399238 | 2052973 |
| AA399239 | 2052974 |
| AA399245 | 2052980 |
| AA399264 | 2052999 |
| AA399265 | 2053000 |
| AA399269 | 2053004 |
| AA399275 | 2053010 |
| AA399281 | 2053016 |
| AA399326 | 2053063 |
| AA399393 | 2053138 |
| AA399433 | 2053246 |
| AA399606 | 2052628 |
| AA399632 | 2052646 |
| AA399633 | 2052647 |
| AA399637 | 2052651 |
| AA400002 | 2053743 |
| AA400010 | 2053751 |
| AA400074 | 2053877 |
| AA400128 | 2053930 |
| AA400213 | 2054084 |
| AA400214 | 2054085 |
| AA400234 | 2054248 |
| AA400247 | 2054313 |
| AA400273 | 2054153 |
| AA400279 | 2054159 |
| AA400281 | 2054161 |
| AA400296 | 2054176 |
| AA400317 | 2054393 |
| AA400329 | 2054254 |
| AA400362 | 2054234 |
| AA400385 | 2054390 |
| AA400389 | 2054322 |
| AA400418 | 2054289 |
| AA400457 | 2054593 |
| AA400482 | 2054353 |
| AA400527 | 2054538 |
| AA400596 | 2054527 |
| AA400658 | 2054547 |
| AA400692 | 2054563 |
| AA400696 | 2054567 |
| AA400732 | 2054620 |
| AA401003 | 2054875 |
| AA401111 | 2054986 |
| AA401137 | 2055027 |
| AA401206 | 2055159 |
| AA401208 | 2055161 |
| AA401237 | 2055126 |
| AA401285 | 2055179 |
| AA401339 | 2053564 |
| AA401342 | 2053758 |
| AA401378 | 2053586 |
| AA401380 | 2053588 |
| AA401409 | 2053634 |
| AA401434 | 2053642 |
| AA401436 | 2053644 |
| AA401441 | 2053649 |
| AA401457 | 2053665 |
| AA401469 | 2053677 |
| AA401470 | 2053765 |
| AA401472 | 2053767 |
| AA401480 | 2053886 |
| AA401482 | 2053888 |
| AA401501 | 2053779 |
| AA401520 | 2053798 |
| AA401521 | 2053799 |
| AA401603 | 2054018 |
| AA401695 | 2057179 |
| AA401784 | 2057268 |
| AA401802 | 2057286 |
| AA401809 | 2055845 |
| AA401826 | 2055915 |
| AA401957 | 2055959 |
| AA401972 | 2056031 |
| AA402000 | 2056025 |
| AA402010 | 2056851 |
| AA402095 | 2056078 |
| AA402115 | 2056106 |
| AA402238 | 2056167 |
| AA402330 | 2056243 |
| AA402352 | 2056264 |
| AA402408 | 2056896 |
| AA402500 | 2056325 |
| AA402721 | 2056477 |
| AA402760 | 2056926 |
| AA402792 | 2056545 |
| AA402874 | 2056628 |
| AA402915 | 2056651 |
| AA402965 | 2056745 |
| AA403051 | 2056800 |
| AA403072 | 2055616 |
| AA403109 | 2055679 |
| AA403120 | 2055638 |
| AA403121 | 2055639 |
| AA403188 | 2055704 |
| AA403235 | 2055760 |
| AA403322 | 2056823 |
| AA404231 | 2058973 |
| AA404269 | 2058993 |
| AA404290 | 2059014 |
| AA404293 | 2059017 |
| AA404338 | 2059063 |
| AA404356 | 2059081 |
| AA404376 | 2059110 |
| AA404444 | 2059186 |
| AA404486 | 2059228 |
| AA404535 | 2059268 |
| AA404615 | 2058827 |
| AA404642 | 2058939 |
| AA404646 | 2058943 |
| AAA04669 | 2058907 |
| AA404722 | 2058925 |
| AA405068 | 2063427 |
| AA405181 | 2063669 |
| AA405532 | 2063043 |
| AA405533 | 2063044 |
| AA405535 | 2063046 |
| AA405543 | 2063054 |
| AA405569 | 2063063 |
| AA405597 | 2063108 |
| AA405640 | 2063132 |
| AA405663 | 2063330 |
| AA405758 | 2063758 |
| AA405769 | 2063875 |
| AA405800 | 2063783 |
| AA405809 | 2063792 |
| AA405892 | 2063893 |
| AA405929 | 2063913 |
| AA405984 | 2064090 |
| AA406019 | 2064002 |
| AA406036 | 2064037 |
| AA406040 | 2064068 |
| AA406059 | 2064042 |
| AA406061 | 2064044 |
| AA406069 | 2064052 |
| AA406070 | 2064053 |
| AA406083 | 2064066 |
| AA406115 | 2064231 |
| AA406122 | 2064166 |
| AA406168 | 2064149 |
| AA406185 | 2064211 |
| AA406191 | 2064217 |
| AA406213 | 2064194 |
| AA406220 | 2064201 |
| AA406226 | 2064207 |
| AA406301 | 2064285 |
| AA406320 | 2064321 |
| AA406402 | 2064403 |
| AA406404 | 2064405 |
| AA406475 | 2064476 |
| AA406478 | 2064584 |
| AA406505 | 2064488 |
| AA406551 | 2064544 |
| AA406565 | 2064558 |
| AA406594 | 2064604 |
| AA410434 | 2069540 |
| AA410437 | 2069543 |
| AA410480 | 2069648 |
| AA410491 | 2069597 |
| AA410517 | 2069623 |
| AA410580 | 2069686 |
| AA410640 | 2069745 |
| AA410700 | 2069805 |
| AA410965 | 2070260 |
| AA410968 | 2070127 |
| AA411201 | 2068819 |
| AA411384 | 2068916 |
| AA411387 | 2068919 |
| AA411585 | 2069118 |
| AA411607 | 2068751 |
| AA411668 | 2069331 |
| AA411682 | 2069345 |
| AA411686 | 2069349 |
| AA411736 | 2069397 |
| AA412046 | 2070760 |
| AA412049 | 2070763 |
| AA412053 | 2070642 |
| AA412229 | 2070799 |
| AA412247 | 2070817 |
| AA412250 | 2070820 |
| AA412302 | 2070873 |
| AA412403 | 2070991 |
| AA412417 | 2071023 |
| AA412435 | 2071005 |
| AA412446 | 2071050 |
| AA422470 | 2071058 |
| AA412477 | 2071065 |
| AA412485 | 2071100 |
| AA412499 | 2071069 |
| AA412500 | 2071070 |
| AA412501 | 2071071 |
| AA412512 | 2071082 |
| AA412643 | 2071222 |
| AA416543 | 2077495 |
| AA416627 | 2077700 |
| AA416664 | 2077598 |
| AA416724 | 2077659 |
| AA416736 | 2077671 |
| AA416759 | 2077713 |
| AA416767 | 2077721 |
| AA416781 | 2077735 |
| AA416782 | 2077736 |
| AA416833 | 2077792 |
| AA416843 | 2077802 |
| AA416970 | 2077069 |
| AA417012 | 2077093 |
| AA417017 | 2077098 |
| AA417031 | 2077130 |
| AA417097 | 2077178 |
| AA417211 | 2077337 |
| AA417246 | 2077345 |
| AA417250 | 2077349 |
| AA417275 | 2077356 |
| AA417280 | 2077361 |
| AA417288 | 2077369 |
| AA417324 | 2077423 |
| AA417344 | 2077426 |
| AA417355 | 2077437 |
| AA417643 | 2079462 |
| AA417713 | 2079550 |
| AA417761 | 2079562 |
| AA417792 | 2079602 |
| AA417805 | 2079589 |
| AA417842 | 2079643 |
| AA417876 | 2079722 |
| AA417895 | 2079714 |
| AA417904 | 2079704 |
| AA417911 | 2079730 |
| AA417915 | 2079734 |
| AA417920 | 2079739 |
| AA417921 | 2079740 |
| AA418000 | 2079819 |
| AA418004 | 2079823 |
| AA418028 | 2079857 |
| AA418061 | 2079935 |
| AA418080 | 2079881 |
| AA418177 | 2079987 |
| AA418383 | 2080230 |
| AA418403 | 2080212 |
| AA418414 | 2080223 |
| AA418418 | 2080237 |
| AA418473 | 2080273 |
| AA418477 | 2080277 |
| AA418545 | 2080345 |
| AA418557 | 2080358 |
| AA418564 | 2080365 |
| AA418628 | 2080438 |
| AA418670 | 2080489 |
| AA418681 | 2080518 |
| AA418685 | 2080522 |
| AA418719 | 2080502 |
| AA418728 | 2080529 |
| AA418744 | 2080636 |
| AA418748 | 2080640 |
| AA418755 | 2080556 |
| AA418770 | 2080571 |
| AA418796 | 2080606 |
| AA418821 | 2080622 |
| AA418824 | 2080625 |
| AA418828 | 2080629 |
| AA418852 | 2080671 |
| AA418903 | 2080722 |
| AA418918 | 2080747 |
| AA418943 | 2080744 |
| AA418988 | 2080807 |
| AA419011 | 2078799 |
| AA419177 | 2078924 |
| AA419263 | 2078976 |
| AA419486 | 2079222 |
| AA419554 | 2079282 |
| AA419612 | 2079339 |
| AA420435 | 2094332 |
| AA420462 | 2094340 |
| AA420523 | 2094420 |
| AA420524 | 2094421 |
| AA420563 | 2094460 |
| AA420571 | 2094468 |
| AA420625 | 2094503 |
| AA420633 | 2094511 |
| AA420645 | 2094561 |
| AA420647 | 2094563 |
| AA420680 | 2094649 |
| AA420690 | 2094569 |
| AA420705 | 2094611 |
| AA420721 | 2094600 |
| AA420758 | 2094637 |
| AA420800 | 2094706 |
| AA420825 | 2094722 |
| AA420826 | 2094723 |
| AA420845 | 2094278 |
| AA420852 | 2094285 |
| AA420989 | 2099822 |
| AA420992 | 2099825 |
| AA420993 | 2099826 |
| AA421001 | 2099834 |
| AA421021 | 2099854 |
| AA421047 | 2099862 |
| AA421054 | 2099869 |
| AA421073 | 2099888 |
| AA421112 | 2099937 |
| AA421170 | 2100125 |
| AA421171 | 2100126 |
| AA421213 | 2100038 |
| AA421230 | 2100072 |
| AA421256 | 2100081 |
| AA421269 | 2100094 |
| AA421270 | 2100095 |
| AA421271 | 2100096 |
| AA421273 | 2100098 |
| AA421274 | 2100099 |
| AA421275 | 2100100 |
| AA421276 | 2100101 |
| AA421284 | 2100109 |
| AA421389 | 2100275 |
| AM421455 | 2100342 |
| AA421510 | 2100352 |
| AA421545 | 2100370 |
| AA421562 | 2100387 |
| AA421682 | 2100499 |
| AA421769 | 2100586 |
| AA421795 | 2100645 |
| AA421850 | 2100809 |
| AA422007 | 2100884 |
| AA422060 | 2100893 |
| AA422139 | 2101007 |
| AA422173 | 2101024 |
| AA423801 | 2102771 |
| AA423943 | 2102913 |
| AA423957 | 2102927 |
| AA424045 | 2103006 |
| AA424109 | 2103079 |
| AA424175 | 2103137 |
| AA424202 | 2103207 |
| AA424243 | 2103195 |
| AA424344 | 2103314 |
| AA424428 | 2103389 |
| AA424456 | 2103444 |
| AA424466 | 2103490 |
| AA424535 | 2103505 |
| AA424543 | 2103513 |
| AA424561 | 2103531 |
| AA424562 | 2103532 |
| AA424648 | 2103645 |
| AA424655 | 2103608 |
| AA424658 | 2103611 |
| AA424706 | 2102756 |
| AA424790 | 2106958 |
| AA424849 | 2106972 |
| AA424900 | 2107005 |
| AA424905 | 2107010 |
| AA424920 | 2107443 |
| AA424950 | 2107038 |
| AA425062 | 2107195 |
| AA425158 | 2107469 |
| AAA25205 | 2105997 |
| AA425212 | 2106120 |
| AA425214 | 2106122 |
| AA425218 | 2106126 |
| AA425298 | 2106108 |
| AA425299 | 2106109 |
| AA425302 | 2106058 |
| AA425305 | 2106061 |
| AA425307 | 2106063 |
| AA425319 | 2106093 |
| AA425382 | 2106165 |
| AA425389 | 2106172 |
| AA425394 | 2106195 |
| AA425404 | 2106160 |
| AA425407 | 2106163 |
| AA425414 | 2106232 |
| AA425436 | 2106201 |
| AA425442 | 2106207 |
| AA425450 | 2106189 |
| AA425460 | 2106216 |
| AA425492 | 2106249 |
| AA425509 | 2106328 |
| AA425526 | 2106267 |
| AA425534 | 2106292 |
| AA425583 | 2106339 |
| AA425613 | 2106369 |
| AA425619 | 2106375 |
| AA425650 | 2106462 |
| AA425664 | 2106439 |
| AA425692 | 2106412 |
| AA425743 | 2106473 |
| AA425749 | 2106451 |
| AA425755 | 2106457 |
| AA425773 | 2106493 |
| AA425826 | 2107646 |
| AA425900 | 2107823 |
| AA425908 | 2107831 |
| AA425947 | 2107735 |
| AA426025 | 2106558 |
| AA426030 | 2106563 |
| AA426038 | 2106526 |
| AA426067 | 2106555 |
| AA426092 | 2106581 |
| AA426110 | 2106625 |
| AA426113 | 2106585 |
| AA426209 | 2107568 |
| AA426212 | 2107615 |
| AA426227 | 2107760 |
| AA426229 | 2107570 |
| AA426235 | 2107576 |
| AA426341 | 2106631 |
| AA426351 | 2106641 |
| AA426380 | 2106652 |
| AA426391 | 2106753 |
| AA426561 | 2106816 |
| AA426578 | 2106806 |
| AA426629 | 2106857 |
| AA427395 | 2112257 |
| AA427400 | 2112167 |
| AA427401 | 2112168 |
| AA427404 | 2112171 |
| AAA27459 | 2111321 |
| AA427471 | 2111333 |
| AA427477 | 2111339 |
| AA427528 | 2112268 |
| AA427561 | 2111429 |
| AA427570 | 2111422 |
| AA427596 | 2111447 |
| AA427719 | 2112179 |
| AA427737 | 2111578 |
| AA427740 | 2111581 |
| AA427866 | 2112185 |
| AA427895 | 2111675 |
| AA427899 | 2111679 |
| AA427927 | 2111689 |
| AA427947 | 2111692 |
| AA427978 | 2112197 |
| AA428008 | 2111736 |
| AA428014 | 2111742 |
| AA428084 | 2111805 |
| AA428096 | 2111773 |
| AAA28143 | 2112093 |
| AA428186 | 2111836 |
| AA428196 | 2111846 |
| AA428216 | 2112108 |
| AA428329 | 2110194 |
| AA428341 | 2110206 |
| AA428415 | 2112229 |
| AA428514 | 2112511 |
| AA428603 | 2112796 |
| AA428659 | 2112807 |
| AA429014 | 2110564 |
| AA429034 | 2110576 |
| AA429036 | 2110578 |
| AA429173 | 2110699 |
| AA429190 | 2110741 |
| AA429293 | 2110817 |
| AAA29297 | 2110821 |
| AA429336 | 2110860 |
| AA429381 | 2111934 |
| AA429422 | 2111942 |
| AA429457 | 2112416 |
| AA429474 | 2112433 |
| AA429493 | 2112612 |
| AA429602 | 2112703 |
| AA429721 | 2112921 |
| AA429904 | 2113076 |
| AA429912 | 2113084 |
| AA430002 | 2113194 |
| AA430032 | 2113206 |
| AA430042 | 2113216 |
| AA430052 | 2113226 |
| AA430110 | 2113284 |
| AA430160 | 2113397 |
| AA430202 | 2113375 |
| AA430205 | 2113378 |
| AA430242 | 2113443 |
| AA430271 | 2113481 |
| AA430304 | 2113514 |
| AA430313 | 2113523 |
| AA430351 | 2110926 |
| AA430367 | 2110942 |
| AA430381 | 2110956 |
| AA430382 | 2110957 |
| AA430400 | 2110974 |
| AA430410 | 2111112 |
| AA430478 | 2111035 |
| AA430506 | 2111096 |
| AA430545 | 2111120 |
| AA430555 | 2111130 |
| AA430597 | 2111953 |
| AA430615 | 2111188 |
| AA430628 | 2112151 |
| AA430718 | 2112377 |
| AA430744 | 2112163 |
| AA430752 | 2111978 |
| AA430754 | 2111980 |
| AA431018 | 2113738 |
| AA431226 | 2114934 |
| AA431401 | 2115109 |
| AA431407 | 2115115 |
| AA431571 | 2115279 |
| AA431579 | 2115287 |
| AA431664 | 2115372 |
| AA431677 | 2115385 |
| AA431721 | 2115429 |
| AA431741 | 2115449 |
| AA431750 | 2115458 |
| AA431753 | 2115461 |
| AA431772 | 2115480 |
| AA431773 | 2115481 |
| AA431836 | 2115544 |
| AA431868 | 2115576 |
| AA431887 | 2115595 |
| AA431964 | 2115672 |
| AA431975 | 2115683 |
| AA431988 | 2115696 |
| AA432030 | 2115738 |
| AA432049 | 2115757 |
| AA432058 | 2115766 |
| AA432064 | 2115772 |
| AA432075 | 2115783 |
| AA432085 | 2115793 |
| AA432096 | 2115804 |
| AA432103 | 2115811 |
| AA432124 | 2114512 |
| AA432134 | 2114522 |
| AA432141 | 2114529 |
| AA432144 | 2114532 |
| AA432203 | 2114591 |
| AA432253 | 2114641 |
| AA432264 | 2114652 |
| AA432270 | 2114658 |
| AA432278 | 2114666 |
| AA432312 | 2114695 |
| AA433848 | 2138762 |
| AA433869 | 2138783 |
| AA433877 | 2138791 |
| AA433880 | 2138794 |
| AA433885 | 2138799 |
| AAA33910 | 2138824 |
| AA433988 | 2138902 |
| AA434028 | 2138942 |
| AA434092 | 2139006 |
| AA434117 | 2139031 |
| AA434286 | 2139200 |
| AA434360 | 2139274 |
| AA434362 | 2139276 |
| AA434388 | 2139302 |
| AA434403 | 2139317 |
| AA434404 | 2139318 |
| AA434454 | 2139368 |
| AA434487 | 2139401 |
| AA434502 | 2139416 |
| AA435565 | 2140479 |
| AA435877 | 2140791 |
| AA435887 | 2140801 |
| AA435894 | 2140808 |
| AA435988 | 2140902 |
| AA436008 | 2140922 |
| AA436031 | 2140945 |
| AA436097 | 2141011 |
| AA436125 | 2141039 |
| AA436142 | 2141056 |
| AA436152 | 2141066 |
| AA436187 | 2141101 |
| AA436197 | 2141111 |
| AA436317 | 2141231 |
| AA436327 | 2141241 |
| AA436377 | 2141291 |
| AA436394 | 2141308 |
| AA436395 | 2141309 |
| AA436472 | 2141386 |
| AA436479 | 2141393 |
| AA436546 | 2141460 |
| AA436573 | 2141487 |
| AA436595 | 2141509 |
| AA436652 | 2141566 |
| AA436730 | 2141644 |
| AA436762 | 2141676 |
| AA436765 | 2141679 |
| AA436769 | 2141683 |
| AA437093 | 2142007 |
| AA437099 | 2142013 |
| AA437107 | 2142021 |
| AA437126 | 2142040 |
| AA437142 | 2142056 |
| AA437212 | 2142126 |
| AA437224 | 2142138 |
| AA437226 | 2142140 |
| AA437241 | 2142155 |
| AA437245 | 2142159 |
| AA437277 | 2142191 |
| AA437304 | 2142218 |
| AA441885 | 2153763 |
| AA441918 | 2153796 |
| AA441933 | 2153811 |
| AA441959 | 2153837 |
| AA442017 | 2153895 |
| AA442095 | 2153973 |
| AA442145 | 2154023 |
| AA442184 | 2154062 |
| AA442287 | 2154165 |
| AA442301 | 2154179 |
| AA442352 | 2154230 |
| AA442372 | 2154250 |
| AA442415 | 2154293 |
| AA442479 | 2154357 |
| AA442517 | 2154395 |
| AA442615 | 2154493 |
| AA442687 | 2154565 |
| AA442829 | 2155504 |
| AA442853 | 2155528 |
| AA442976 | 2155651 |
| AA442991 | 2155666 |
| AA443004 | 2155679 |
| AA443024 | 2155699 |
| AA443082 | 2155757 |
| AA443090 | 2155765 |
| AA443094 | 2155769 |
| AA443116 | 2155791 |
| AA443140 | 2155815 |
| AA443147 | 2155822 |
| AA443152 | 2155827 |
| AA443154 | 2155829 |
| AA443177 | 2155852 |
| AA443184 | 2155859 |
| AA443242 | 2155917 |
| AA443286 | 2155961 |
| AA443300 | 2155975 |
| AA443357 | 2156032 |
| AA443447 | 2156122 |
| AA443463 | 2156138 |
| AA443602 | 2156277 |
| AA443624 | 2158299 |
| AA443638 | 2156313 |
| AA443649 | 2156324 |
| AA443659 | 2156334 |
| AA443688 | 2156363 |
| AA443698 | 2156373 |
| AA443706 | 2156381 |
| AA443750 | 2156425 |
| AA443757 | 2156432 |
| AA443790 | 2156465 |
| AA443806 | 2156481 |
| AA443823 | 2156498 |
| AA443829 | 2156504 |
| AA443832 | 2156507 |
| AA443834 | 2156509 |
| AA443870 | 2156545 |
| AA443903 | 2156578 |
| AA443936 | 2156611 |
| AA443938 | 2156613 |
| AA443946 | 2156621 |
| AA443950 | 2156625 |
| AA443958 | 2156633 |
| AA443968 | 2156641 |
| AA443967 | 2156642 |
| AA443969 | 2156644 |
| AA444051 | 2156726 |
| AA444072 | 2156747 |
| AA444074 | 2156749 |
| AA444083 | 2156758 |
| AA444093 | 2156768 |
| AA444111 | 2156786 |
| AA444386 | 2157051 |
| AA445955 | 2158620 |
| AA446031 | 2158696 |
| AA446071 | 2158736 |
| AA446103 | 2158768 |
| AA446116 | 2158781 |
| AA446120 | 2158785 |
| AA446149 | 2158814 |
| AA446298 | 2158963 |
| AA446316 | 2158981 |
| AA446322 | 2158987 |
| AA446366 | 2159031 |
| AA446446 | 2159111 |
| AA446451 | 2159116 |
| AA446462 | 2159127 |
| AA446479 | 2159144 |
| AA446639 | 2159304 |
| AA446643 | 2159308 |
| AA446658 | 2159323 |
| AA446690 | 2159355 |
| AA446716 | 2159381 |
| AA446743 | 2159408 |
| AA446748 | 2159413 |
| AA446759 | 2159424 |
| AA446760 | 2159425 |
| AA446789 | 2159454 |
| AA446839 | 2159504 |
| AA446864 | 2159529 |
| AA446866 | 2159531 |
| AA446877 | 2159542 |
| AA446882 | 2159547 |
| AA446885 | 2159550 |
| AA446906 | 2159571 |
| AA446910 | 2159575 |
| AA446943 | 2159608 |
| AA446958 | 2159623 |
| AA447018 | 2159683 |
| AA447021 | 2159686 |
| AA447069 | 2159734 |
| AA447079 | 2159744 |
| AA447116 | 2159781 |
| AA447189 | 2159854 |
| AA447254 | 2159919 |
| AA447322 | 3025408 |
| AA447343 | 2161013 |
| AA447349 | 2161019 |
| AA447401 | 2161071 |
| AA447433 | 2161103 |
| AA447476 | 2161146 |
| AA447503 | 2161173 |
| AA447504 | 2161174 |
| AA447514 | 2161184 |
| AA447522 | 2161192 |
| AA447527 | 2161197 |
| AA447531 | 2161201 |
| AA447547 | 2161217 |
| AA447559 | 2161229 |
| AA447561 | 2161231 |
| AA447563 | 2161233 |
| AA447574 | 2161244 |
| AA447585 | 2161255 |
| AA447592 | 2161262 |
| AA447608 | 2161278 |
| AA447662 | 2161332 |
| AA447700 | 2161370 |
| AA447724 | 2161394 |
| AA447731 | 2161401 |
| AA447740 | 2161410 |
| AA447742 | 2161412 |
| AA447743 | 2161413 |
| AA447744 | 2161414 |
| AA447751 | 2161421 |
| AA447761 | 2161431 |
| AA447764 | 2161434 |
| AA447815 | 2161485 |
| AA447885 | 2161555 |
| AA447948 | 2161618 |
| AA447986 | 2161656 |
| AA448036 | 2161706 |
| AA448118 | 2161788 |
| AA448160 | 2161830 |
| AA448172 | 2161842 |
| AA448184 | 2161854 |
| AA448186 | 2161856 |
| AA448194 | 2161864 |
| AA448195 | 2161865 |
| AA448251 | 2161921 |
| AA448257 | 2161927 |
| AA448270 | 2161940 |
| AA448283 | 2161953 |
| AA448289 | 2161959 |
| AA448390 | 2162060 |
| AA448394 | 2162064 |
| AA448396 | 2162066 |
| AA448400 | 2162070 |
| AA448402 | 2162072 |
| AA448484 | 2162154 |
| AA448486 | 2162156 |
| AA448514 | 2162184 |
| AA448526 | 2162196 |
| AA448531 | 2162201 |
| AA448542 | 2162212 |
| AA448599 | 2162269 |
| AA448614 | 2162284 |
| AA448617 | 2162287 |
| AA448664 | 2162334 |
| AA448672 | 2162342 |
| AA448676 | 2162346 |
| AA448795 | 2162465 |
| AA448825 | 2162495 |
| AA448855 | 2162525 |
| AA448882 | 2162552 |
| AA448950 | 2162970 |
| AA449004 | 2163024 |
| AA449037 | 2163057 |
| AA449048 | 2163068 |
| AA449054 | 2163074 |
| AA449085 | 2163105 |
| AA449118 | 2163138 |
| AA449120 | 2163140 |
| AA449228 | 2162691 |
| AA449300 | 2163149 |
| AA449305 | 2163154 |
| AA449345 | 2163194 |
| AA449362 | 2163211 |
| AA449435 | 2162826 |
| AA449520 | 2163270 |
| AA449544 | 2163294 |
| AA449604 | 2163354 |
| AA449632 | 2163382 |
| AA449644 | 2163394 |
| AA449649 | 2163399 |
| AA449657 | 2163407 |
| AA449663 | 2163413 |
| AA449686 | 2163436 |
| AA449745 | 2163495 |
| AA449762 | 2163512 |
| AA449823 | 2163573 |
| AA449837 | 2163587 |
| AA449889 | 2163639 |
| AA449891 | 2163641 |
| AA449907 | 2163657 |
| AA449922 | 2163672 |
| AA449940 | 2163690 |
| AA450051 | 2163801 |
| AA450120 | 2163870 |
| AA450168 | 2163918 |
| AA450205 | 2163955 |
| AA450265 | 2164015 |
| AA451633 | 2165302 |
| AA451733 | 2165402 |
| AA451807 | 2165476 |
| AA451863 | 2165532 |
| AA451886 | 2165555 |
| AA451890 | 2165559 |
| AA451988 | 2165657 |
| AA452012 | 2165681 |
| AA452027 | 2165696 |
| AA452095 | 2165764 |
| AA452113 | 2165782 |
| AA452120 | 2165789 |
| AA452125 | 2165794 |
| AA452139 | 2165808 |
| AA452177 | 2165846 |
| AA452200 | 2165869 |
| AA452278 | 2165947 |
| AA452288 | 2165957 |
| AA452352 | 2166021 |
| AA452412 | 2166081 |
| AA452542 | 2166211 |
| AA452566 | 2166235 |
| AA452578 | 2166247 |
| AA452722 | 2166391 |
| AA452897 | 2166566 |
| AA452909 | 2166578 |
| AA452920 | 2166589 |
| AA452933 | 2166602 |
| AA452955 | 2166624 |
| AA452966 | 2166635 |
| AA452999 | 2166668 |
| AA453015 | 2166684 |
| AA453040 | 2166709 |
| AA453085 | 2166754 |
| AA453105 | 2166774 |
| AA453134 | 2166803 |
| AA453256 | 2166925 |
| AA453260 | 2166929 |
| AA453261 | 2166930 |
| AA453273 | 2166942 |
| AA453310 | 2166979 |
| AA453333 | 2167002 |
| AA453345 | 2167014 |
| AA453352 | 2167021 |
| AA453433 | 2167102 |
| AA453437 | 2167106 |
| AA453441 | 2167110 |
| AA453445 | 2167114 |
| AA453465 | 2167134 |
| AA453466 | 2167135 |
| AA453478 | 2167147 |
| AA453486 | 2167155 |
| AA453487 | 2167156 |
| AA453518 | 2167187 |
| AA453526 | 2167195 |
| AA453531 | 2167200 |
| AA453550 | 2167219 |
| AA453555 | 2167224 |
| AA453562 | 2167231 |
| AA453585 | 2167254 |
| AA453609 | 2167278 |
| AA453612 | 2167281 |
| AA453616 | 2167285 |
| AA453651 | 2167320 |
| AA453679 | 2167348 |
| AA453713 | 2167382 |
| AA453714 | 2167383 |
| AA453719 | 2167388 |
| AA453769 | 2167438 |
| AA453783 | 2167452 |
| AA453784 | 2167453 |
| AA453787 | 2167456 |
| AA453795 | 2167464 |
| AA453802 | 2167471 |
| AA453832 | 2167501 |
| AA453849 | 2167518 |
| AA453863 | 2167532 |
| AA453900 | 2167569 |
| AA453924 | 2167593 |
| AA453926 | 2167595 |
| AA453993 | 2167662 |
| AA453996 | 2167665 |
| AA453997 | 2167666 |
| AA454018 | 2167687 |
| AA454027 | 2167696 |
| AA454065 | 2167734 |
| AA454080 | 2167749 |
| AA454082 | 2167751 |
| AA454085 | 2167754 |
| AA454098 | 2167767 |
| AA454149 | 2167818 |
| AA454155 | 2167824 |
| AA454165 | 2167834 |
| AA454170 | 2167839 |
| AA454186 | 2167855 |
| AA454187 | 2167856 |
| AA454208 | 2167877 |
| AA454215 | 2167884 |
| AA454218 | 2167887 |
| AA454222 | 2167891 |
| AA454554 | 2177330 |
| AA454557 | 2177333 |
| AA454562 | 2177338 |
| AA454570 | 2177346 |
| AA454585 | 2177361 |
| AA454597 | 2177373 |
| AA454634 | 2177410 |
| AA454652 | 2177428 |
| AA454654 | 2177430 |
| AA454702 | 2177478 |
| AA454756 | 2177532 |
| AA454844 | 2177620 |
| AA454862 | 2177638 |
| AA454912 | 2177688 |
| AA454921 | 2177697 |
| AA454925 | 2177701 |
| AA454928 | 2177704 |
| AA454947 | 2177723 |
| AA454949 | 2177725 |
| AA454962 | 2177738 |
| AA454963 | 2177739 |
| AA454986 | 2177762 |
| AA455020 | 2177796 |
| AA455042 | 2177818 |
| AA455056 | 2177832 |
| AA455062 | 2177838 |
| AA455087 | 2177863 |
| AA455096 | 2177872 |
| AA455126 | 2177902 |
| AA455130 | 2177906 |
| AA455151 | 2177927 |
| AA455164 | 2177940 |
| AA455197 | 2177973 |
| AA455227 | 2178003 |
| AA455235 | 2178011 |
| AA455237 | 2178013 |
| AA455242 | 2178018 |
| AA455275 | 2178051 |
| AA455299 | 2178075 |
| AA455358 | 2178134 |
| AA455369 | 2178145 |
| AA455475 | 2178251 |
| AA455482 | 2178258 |
| AA455497 | 2178273 |
| AA455519 | 2178295 |
| AA455521 | 2178297 |
| AA455565 | 2178341 |
| AA455652 | 2178428 |
| AA455654 | 2178430 |
| AA455659 | 2178435 |
| AA455668 | 2178444 |
| AA455689 | 2178465 |
| AA455714 | 2178490 |
| AA455786 | 2178562 |
| AA455825 | 2178601 |
| AA455894 | 2178670 |
| AA455904 | 2178680 |
| AA455917 | 2178693 |
| AA455931 | 2178707 |
| AA455938 | 2178714 |
| AA455940 | 2178716 |
| AA455980 | 2178756 |
| AA455988 | 2178764 |
| AA456008 | 2178784 |
| AA456035 | 2178811 |
| AA456078 | 2178854 |
| AA456130 | 2179340 |
| AA456135 | 2179345 |
| AA456143 | 2179353 |
| AA456148 | 2179358 |
| AA456246 | 2179456 |
| AA456271 | 2179481 |
| AA456298 | 2179508 |
| AA456299 | 2179509 |
| AA456321 | 2179531 |
| AA456331 | 2178907 |
| AA456344 | 2178920 |
| AA456372 | 2178948 |
| AA456377 | 2178953 |
| AA456390 | 2178966 |
| AA456413 | 2178989 |
| AA456419 | 2178995 |
| AA456569 | 2179145 |
| AA456611 | 2179187 |
| AA456616 | 2179192 |
| AA456628 | 2179204 |
| AA456695 | 2179271 |
| AA456717 | 2179293 |
| AA456721 | 2179297 |
| AA456737 | 2179313 |
| AA456818 | 2179538 |
| AA456821 | 2179541 |
| AA456827 | 2179547 |
| AA456833 | 2179553 |
| AA456886 | 2179606 |
| AA456931 | 2179651 |
| AA456951 | 2179671 |
| AA456968 | 2179688 |
| AA456975 | 2179695 |
| AA457021 | 2179741 |
| AA457041 | 2179761 |
| AA457082 | 2179802 |
| AA457092 | 2179812 |
| AA457138 | 2179858 |
| AA457172 | 2179892 |
| AA457235 | 2179955 |
| AA457365 | 2180085 |
| AA457474 | 2180194 |
| AA457517 | 2180237 |
| AA457528 | 2180248 |
| AA457529 | 2180249 |
| AA457576 | 2180296 |
| AA457675 | 2180395 |
| AA457681 | 2180401 |
| AA457696 | 2180416 |
| AA457717 | 2180437 |
| AA457720 | 2180440 |
| AA457725 | 2180445 |
| AA457737 | 2180457 |
| AA457767 | 2180487 |
| AA458487 | 2183394 |
| AA458491 | 2183398 |
| AA458514 | 2183421 |
| AA458516 | 2183423 |
| AA458528 | 2183435 |
| AA458549 | 2183456 |
| AA458551 | 2183458 |
| AA458578 | 2183485 |
| AA458646 | 2183553 |
| AA458747 | 2183654 |
| AA458761 | 2183668 |
| AA458770 | 2183677 |
| AA458804 | 2183711 |
| AA458827 | 2183734 |
| AA458921 | 2183828 |
| AA458943 | 2183850 |
| AA458959 | 2183866 |
| AA459008 | 2183915 |
| AA459100 | 2184007 |
| AA459106 | 2184013 |
| AA459110 | 2184017 |
| AA459123 | 2184030 |
| AA459167 | 2184074 |
| AA459197 | 2184104 |
| AA459208 | 2184115 |
| AA459210 | 2184117 |
| AA459244 | 2184151 |
| AA459292 | 2134199 |
| AA459293 | 2184200 |
| AA459318 | 2184225 |
| AA459321 | 2184228 |
| AA459383 | 2184290 |
| AA459400 | 2184307 |
| AA459455 | 2184362 |
| AA459472 | 2184379 |
| AA459476 | 2184383 |
| AA459517 | 2184424 |
| AA459519 | 2184426 |
| AA459528 | 2184435 |
| AA459617 | 2184524 |
| AA459632 | 2184539 |
| AA459651 | 2184558 |
| AA459652 | 2184559 |
| AA459679 | 2184586 |
| AA459692 | 2184599 |
| AA459702 | 2184609 |
| AA459750 | 2184657 |
| AA459753 | 2184660 |
| AA459761 | 2184668 |
| AA459765 | 2184672 |
| AA459831 | 2184738 |
| AA459857 | 2184764 |
| AA459869 | 2184776 |
| AA459871 | 2184778 |
| AA459909 | 2183355 |
| AA459917 | 2184801 |
| AA459941 | 2184825 |
| AA459945 | 2184829 |
| AA459980 | 2184864 |
| AA459983 | 2184867 |
| AA460115 | 2185500 |
| AA460120 | 2185505 |
| AA460251 | 2185067 |
| AA460274 | 2185090 |
| AA460282 | 2185098 |
| AA460285 | 2185101 |
| AA460286 | 2185102 |
| AA460289 | 2185105 |
| AA460291 | 2185107 |
| AA460299 | 2185115 |
| AA460310 | 2185126 |
| AA460313 | 2185129 |
| AA460328 | 2185144 |
| AA460346 | 2185559 |
| AA460353 | 2185566 |
| AA460422 | 2185168 |
| AA460440 | 2185186 |
| AA460463 | 2185209 |
| AA460511 | 2185631 |
| AA460521 | 2185641 |
| AA460530 | 2185650 |
| AA460557 | 2185677 |
| AA460571 | 2185691 |
| AA460587 | 2185707 |
| AA460649 | 2185769 |
| AA460675 | 2185795 |
| AA460685 | 2185805 |
| AA460702 | 2185822 |
| AA460719 | 2185839 |
| AA460729 | 2185849 |
| AA460755 | 2185875 |
| AA460777 | 2185897 |
| AA460816 | 2185936 |
| AA460827 | 2185947 |
| AA460849 | 2185969 |
| AA460930 | 2186050 |
| AA460952 | 2186072 |
| AA460961 | 2186081 |
| AA460963 | 2186083 |
| AA460967 | 2186087 |
| AA460969 | 2186089 |
| AA460986 | 2186106 |
| AA461015 | 2186135 |
| AA461082 | 2186202 |
| AA461086 | 2186206 |
| AA461092 | 2186212 |
| AA461119 | 2186239 |
| AA461128 | 2186248 |
| AA461174 | 2186294 |
| AA461222 | 2186342 |
| AA461260 | 2186380 |
| AA461307 | 2186427 |
| AA461311 | 2186431 |
| AA461320 | 2186440 |
| AA461467 | 2185331 |
| AA461506 | 2185370 |
| AA461507 | 2185371 |
| AA461508 | 2185372 |
| AA461513 | 2185377 |
| AA461522 | 2185386 |
| AA461548 | 2185412 |
| AA461605 | 2185469 |
| AA463188 | 2188072 |
| AA463198 | 2188082 |
| AA463200 | 2188084 |
| AA463225 | 2188109 |
| AA463248 | 2188132 |
| AA463444 | 2188328 |
| AA463445 | 2188329 |
| AA463449 | 2188333 |
| AA463463 | 2188347 |
| AA463469 | 2188353 |
| AA463483 | 2188367 |
| AA463500 | 2188384 |
| AA463533 | 2188417 |
| AA463627 | 2188511 |
| AA463635 | 2188519 |
| AA463643 | 2188527 |
| AA463761 | 2188645 |
| AA463822 | 2188706 |
| AA463924 | 2188808 |
| AA463932 | 2188816 |
| AA463982 | 2188866 |
| AA464068 | 2188952 |
| AA464133 | 2189017 |
| AA464141 | 2189025 |
| AA464143 | 2189027 |
| AA464160 | 2189044 |
| AA464163 | 2189047 |
| AA464338 | 2189222 |
| AA464522 | 2189406 |
| AA464531 | 2189415 |
| AA464541 | 2189425 |
| AA464568 | 2189452 |
| AA464580 | 2189464 |
| AA464598 | 2189482 |
| AA464600 | 2189484 |
| AA464615 | 2189499 |
| AA464617 | 2189501 |
| AA464623 | 2189507 |
| AA464689 | 2189573 |
| AA464700 | 2189584 |
| AA464755 | 2189639 |
| AA464763 | 2189647 |
| AA464861 | 2189745 |
| AA464880 | 2189764 |
| AA464935 | 2189819 |
| AA464941 | 2189825 |
| AA464952 | 2189836 |
| AA464979 | 2189863 |
| AA464995 | 2189879 |
| AA465090 | 2191257 |
| AA465116 | 2191283 |
| AA465148 | 2191315 |
| AA465166 | 2191333 |
| AA465168 | 2191335 |
| AA465180 | 2191347 |
| AA465193 | 2191360 |
| AA465194 | 2191361 |
| AA465223 | 2191390 |
| AA465238 | 2191405 |
| AA465258 | 2191425 |
| AA465293 | 2191460 |
| AA465338 | 2191505 |
| AA465340 | 2191507 |
| AA465353 | 2191520 |
| AA465387 | 2191554 |
| AA465424 | 2191591 |
| AA465450 | 2191617 |
| AA465494 | 2191661 |
| AA465495 | 2191662 |
| AA465509 | 2191676 |
| AA465593 | 2191760 |
| AA465598 | 2191765 |
| AA465614 | 2191781 |
| AA465692 | 2191859 |
| AA465697 | 2191364 |
| AA465723 | 2191245 |
| AA467748 | 2194282 |
| AA467750 | 2194284 |
| AA467761 | 2194295 |
| AA467864 | 2194398 |
| AA467869 | 2194403 |
| AA467974 | 2194508 |
| AA468151 | 2194685 |
| AA468167 | 2194701 |
| AA468335 | 2194869 |
| AA468404 | 2194938 |
| AA468411 | 2194945 |
| AA468504 | 2195038 |
| AA468565 | 2195099 |
| AA468575 | 2195109 |
| AA468624 | 2195158 |
| AA468760 | 2195294 |
| AA468839 | 2195373 |
| AA469129 | 2195663 |
| AA469151 | 2195685 |
| AA469199 | 2195733 |
| AA469238 | 2195772 |
| AA469303 | 2195837 |
| AA469304 | 2195838 |
| AA469345 | 2195879 |
| AA469348 | 2195882 |
| AA469406 | 2195940 |
| AA469432 | 2194227 |
| AA469922 | 2197231 |
| AA469926 | 2197235 |
| AA469939 | 2197248 |
| AA469966 | 2197275 |
| AA469979 | 2197288 |
| AA470051 | 2197360 |
| AA470073 | 2197382 |
| AA470079 | 2197388 |
| AA470575 | 2197884 |
| AA470602 | 2197911 |
| AA470662 | 2197971 |
| AA470690 | 2197999 |
| AA470829 | 2198138 |
| AA470833 | 2198142 |
| AA471025 | 2198334 |
| AA471056 | 2198365 |
| AA471070 | 2198379 |
| AA476207 | 2204418 |
| AA476234 | 2204445 |
| AA476240 | 2204451 |
| AA476257 | 2204468 |
| AA476263 | 2204474 |
| AA476360 | 2204571 |
| AA476392 | 2204603 |
| AA476487 | 2204698 |
| AA476502 | 2204713 |
| AA476509 | 2204720 |
| AA476524 | 2204735 |
| AA476543 | 2204754 |
| AA476576 | 2204787 |
| AA476586 | 2204797 |
| AA476648 | 2204859 |
| AA476679 | 2204890 |
| AA476725 | 2204938 |
| AA476756 | 2204967 |
| AA476758 | 2204969 |
| AA476961 | 2205172 |
| AA477075 | 2205286 |
| AA477173 | 2205857 |
| AA477227 | 2205302 |
| AA477283 | 2205917 |
| AA477288 | 2205922 |
| AA477397 | 2206031 |
| AA477400 | 2206034 |
| AA477579 | 2206213 |
| AA477597 | 2206231 |
| AA477683 | 2206317 |
| AA477698 | 2206332 |
| AA477728 | 2206362 |
| AA477747 | 2206381 |
| AA477803 | 2206437 |
| AA477848 | 2206482 |
| AA477856 | 2206490 |
| AA478036 | 2206670 |
| AA478066 | 2206700 |
| AA478258 | 2206892 |
| AA478268 | 2206902 |
| AA478298 | 2206932 |
| AA478415 | 2207049 |
| AA478436 | 2207070 |
| AA478452 | 2207086 |
| AA478470 | 2207104 |
| AA478479 | 2207113 |
| AA478481 | 2207115 |
| AA478514 | 2207148 |
| AA478518 | 2207152 |
| AA478522 | 2207156 |
| AA478539 | 2207173 |
| AA478542 | 2207176 |
| AA478606 | 2207240 |
| AA478627 | 2207261 |
| AA478647 | 2207281 |
| AA478842 | 2207476 |
| AA478891 | 2207525 |
| AA478931 | 2207565 |
| AA478952 | 2207586 |
| AA478965 | 2207599 |
| AA479058 | 2207614 |
| AA479100 | 2207656 |
| AA479133 | 2207689 |
| AA479151 | 2207707 |
| AA479154 | 2207710 |
| AA479167 | 2207723 |
| AA479276 | 2207832 |
| AA479280 | 2207836 |
| AA479284 | 2207840 |
| AA479287 | 2207843 |
| AA479313 | 2207869 |
| AA479327 | 2207883 |
| AA479342 | 2207898 |
| AA479351 | 2207907 |
| AA479363 | 2207919 |
| AA479444 | 2208000 |
| AA479494 | 2208050 |
| AA479498 | 2208054 |
| AA479512 | 2208068 |
| AA479614 | 2205500 |
| AA479646 | 2205532 |
| AA479689 | 2205575 |
| AA479781 | 2205667 |
| AA479795 | 2205681 |
| AA479809 | 2205695 |
| AA479860 | 2205746 |
| AA479906 | 2204388 |
| AA479912 | 2204394 |
| AA479928 | 2204410 |
| AA479962 | 2208113 |
| AA479967 | 2208118 |
| AA479976 | 2208127 |
| AA480051 | 2208202 |
| AA480124 | 2208275 |
| AA480202 | 2208353 |
| AA480226 | 2208377 |
| AA480282 | 2208433 |
| AA480420 | 2208571 |
| AA480505 | 2208656 |
| AA480529 | 2208680 |
| AA480551 | 2208702 |
| AA480820 | 2210372 |
| AA480844 | 2210396 |
| AA480863 | 2210415 |
| AA480870 | 2210422 |
| AA480906 | 2210458 |
| AA481001 | 2210553 |
| AA481045 | 2210597 |
| AA481047 | 2210599 |
| AA481052 | 2210604 |
| AA481078 | 2210630 |
| AA481135 | 2210687 |
| AA481144 | 2210696 |
| AA481152 | 2210704 |
| AA481158 | 2210710 |
| AA481256 | 2210808 |
| AA481303 | 2210855 |
| AA481316 | 2210868 |
| AA481355 | 2210907 |
| AA481397 | 2210949 |
| AA481437 | 2210989 |
| AA481438 | 2210990 |
| AA481464 | 2211016 |
| AA481476 | 2211028 |
| AA481481 | 2211033 |
| AA481491 | 2211043 |
| AA481571 | 2211123 |
| AA481738 | 2211290 |
| AA481751 | 2211303 |
| AA481754 | 2211306 |
| AA481757 | 2211309 |
| AA481770 | 2211322 |
| AA481788 | 2211340 |
| AA481806 | 2211358 |
| AA481979 | 2209657 |
| AA482007 | 2209685 |
| AA482119 | 2209797 |
| AA482127 | 2209805 |
| AA482150 | 2209828 |
| AA482198 | 2209876 |
| AA482228 | 2209906 |
| AA482285 | 2209963 |
| AA482324 | 2210002 |
| AA482340 | 2210018 |
| AA482403 | 2210081 |
| AA482430 | 2210108 |
| AA482434 | 2210112 |
| AA482522 | 2210200 |
| AA482539 | 2210217 |
| AA482654 | 2210332 |
| AA482724 | 2211569 |
| AA482853 | 2211698 |
| AA482884 | 2211729 |
| AA483008 | 2211853 |
| AA483013 | 2211858 |
| AA483044 | 2211889 |
| AA483214 | 2212027 |
| AA483289 | 2212102 |
| AA483565 | 2212378 |
| AA483635 | 2212448 |
| AA483857 | 2212670 |
| AA483887 | 2212700 |
| AA484104 | 2212917 |
| AA484284 | 2213097 |
| AA484568 | 2213381 |
| AA484711 | 2211505 |
| AA484751 | 2214136 |
| AA484752 | 2214137 |
| AA484821 | 2214054 |
| AA485018 | 2214237 |
| AA485024 | 2214243 |
| AA485052 | 2214271 |
| AA485065 | 2214284 |
| AA485102 | 2214321 |
| AA485140 | 2214359 |
| AA485201 | 2214420 |
| AA485214 | 2214433 |
| AA485216 | 2214435 |
| AA485217 | 2214436 |
| AA485249 | 2214468 |
| AA485265 | 2214484 |
| AA485303 | 2214522 |
| AA485333 | 2214552 |
| AA485351 | 2214570 |
| AA485352 | 2214571 |
| AA485357 | 2214576 |
| AA485365 | 2214584 |
| AA485373 | 2214592 |
| AA485380 | 2214599 |
| AA485427 | 2214646 |
| AA485429 | 2214648 |
| AA485441 | 2214660 |
| AA485453 | 2214672 |
| AA485467 | 2214686 |
| AA485508 | 2214727 |
| AA485653 | 2214872 |
| AA485675 | 2214894 |
| AA485676 | 2214895 |
| AA485688 | 2214907 |
| AA485714 | 2214933 |
| AA485748 | 2214967 |
| AA485749 | 2214968 |
| AA485767 | 2214986 |
| AA485773 | 2214992 |
| AA485853 | 2215072 |
| AA485861 | 2215080 |
| AA485898 | 2215117 |
| AA485906 | 2216130 |
| AA485922 | 2216146 |
| AA485928 | 2216152 |
| AA485935 | 2216159 |
| AA485983 | 2216199 |
| AA485996 | 2216212 |
| AA486041 | 2216257 |
| AA486071 | 2216287 |
| AA486082 | 2216298 |
| AA486084 | 2216300 |
| AA486145 | 2216361 |
| AA486200 | 2216416 |
| AA486261 | 2216477 |
| AA486281 | 2216497 |
| AA486288 | 2216504 |
| AA486300 | 2216516 |
| AA486370 | 2215176 |
| AA486403 | 2216567 |
| AA486412 | 2216576 |
| AA486413 | 2216577 |
| AA486430 | 2216594 |
| AA486435 | 2216599 |
| AA486444 | 2216608 |
| AA486445 | 2216609 |
| AA486471 | 2216635 |
| AA486516 | 2216680 |
| AA486533 | 2216697 |
| AA486538 | 2216702 |
| AA486551 | 2216715 |
| AA486561 | 2216725 |
| AA486619 | 2216783 |
| AA486628 | 2216792 |
| AA486669 | 2216833 |
| AA486723 | 2216887 |
| AA486741 | 2216905 |
| AA486765 | 2216929 |
| AA486837 | 2217001 |
| AA486838 | 2217002 |
| AA486862 | 2217026 |
| AA486864 | 2217028 |
| AA487020 | 2217184 |
| AA487031 | 2217195 |
| AA487046 | 2217210 |
| AA487146 | 2217310 |
| AA487192 | 2217356 |
| AA487203 | 2217367 |
| AA487206 | 2217370 |
| AA487213 | 2217377 |
| AA487215 | 2217379 |
| AA487261 | 2217425 |
| AA487382 | 2217546 |
| AA487385 | 2217549 |
| AA487432 | 2217596 |
| AA487448 | 2217612 |
| AA487457 | 2217621 |
| AA487463 | 2217627 |
| AA487465 | 2217629 |
| AA487468 | 2217632 |
| AA487503 | 2217667 |
| AA487505 | 2217669 |
| AA487552 | 2217716 |
| AA487556 | 2217720 |
| AA487560 | 2217724 |
| AA487561 | 2217725 |
| AA487608 | 2217772 |
| AA487650 | 2217814 |
| AA487674 | 2217838 |
| AA487676 | 2217840 |
| AA487680 | 2217844 |
| AA487747 | 2217911 |
| AA487754 | 2215185 |
| AA487902 | 2215333 |
| AA487914 | 2215345 |
| AA487934 | 2215365 |
| AA488014 | 2215445 |
| AA488036 | 2215467 |
| AA488064 | 2215495 |
| AA488175 | 2215606 |
| AA488227 | 2215658 |
| AA488238 | 2215669 |
| AA488280 | 2215711 |
| AA488324 | 2215755 |
| AA488367 | 2215798 |
| AA488445 | 2215876 |
| AA488468 | 2215899 |
| AA488481 | 2215912 |
| AA488567 | 2215998 |
| AA488588 | 2216019 |
| AA488592 | 2216023 |
| AA488604 | 2216035 |
| AA488626 | 2216057 |
| AA488633 | 2216064 |
| AA488676 | 2216107 |
| AA488732 | 2218334 |
| AA488744 | 2218346 |
| AA488784 | 2218386 |
| AA488809 | 2218411 |
| AA488865 | 2218467 |
| AA488892 | 2218494 |
| AA488893 | 2218495 |
| AA488954 | 2218556 |
| AA488976 | 2218578 |
| AA488979 | 2218581 |
| AA488986 | 2218588 |
| AA489000 | 2218602 |
| AA489012 | 2218614 |
| AA489016 | 2218618 |
| AA489020 | 2218622 |
| AA489032 | 2218634 |
| AA489040 | 2218642 |
| AA489073 | 2218675 |
| AA489074 | 2218676 |
| AA489121 | 2218723 |
| AA489124 | 2218726 |
| AA489160 | 2218762 |
| AA489173 | 2218775 |
| AA489194 | 2218796 |
| AA489219 | 2218821 |
| AA489247 | 2218849 |
| AA489261 | 2218863 |
| AA489323 | 2218925 |
| AA489343 | 2218945 |
| AA489410 | 2219012 |
| AA489433 | 2219035 |
| AA489447 | 2219049 |
| AA489478 | 2219080 |
| AA489498 | 2219100 |
| AA489569 | 2219171 |
| AA489611 | 2219213 |
| AA489639 | 2219241 |
| AA489647 | 2219249 |
| AA489662 | 2219264 |
| AA489666 | 2219268 |
| AA489670 | 2219272 |
| AA489681 | 2219283 |
| AA489696 | 2219298 |
| AA489729 | 2219331 |
| AA489743 | 2219345 |
| AA489752 | 2219354 |
| AA489782 | 2220666 |
| AA489791 | 2220675 |
| AA489921 | 2220796 |
| AA489965 | 2220840 |
| AA490044 | 2220919 |
| AA490047 | 2220922 |
| AA490058 | 2220933 |
| AA490213 | 2219395 |
| AA490240 | 2219422 |
| AA490263 | 2219436 |
| AA490264 | 2219437 |
| AA490390 | 2219563 |
| AA490456 | 2219629 |
| AA490490 | 2219663 |
| AA490522 | 2219695 |
| AA490561 | 2219734 |
| AA490605 | 2219778 |
| AA490609 | 2219782 |
| AA490611 | 2219784 |
| AA490635 | 2219808 |
| AA490734 | 2219907 |
| AA490802 | 2219975 |
| AA490893 | 2220066 |
| AA490910 | 2220083 |
| AA490945 | 2220118 |
| AA490975 | 2220148 |
| AA490985 | 2220158 |
| AA490987 | 2220160 |
| AA491041 | 2220214 |
| AA491081 | 2220254 |
| AA491206 | 2220379 |
| AA491227 | 2220400 |
| AA491249 | 2220422 |
| AA491261 | 2220434 |
| AA491265 | 2220438 |
| AA491272 | 2220445 |
| AA491302 | 2220475 |
| AA491312 | 2220485 |
| AA491370 | 2220543 |
| AA491866 | 2221428 |
| AA491983 | 2221545 |
| AA492026 | 2221588 |
| AA492035 | 2221597 |
| AA492038 | 2221600 |
| AA492042 | 2221604 |
| AA492143 | 2221705 |
| AA492192 | 2221754 |
| AA492219 | 2221781 |
| AA492256 | 2221818 |
| AA492272 | 2221834 |
| AA492280 | 2221842 |
| AA492390 | 2221952 |
| AA492392 | 2221954 |
| AA493183 | 2223024 |
| AA493222 | 2223063 |
| AA493331 | 2223172 |
| AA493448 | 2223289 |
| AA493512 | 2223353 |
| AA493551 | 2223392 |
| AA493742 | 2223583 |
| AA493956 | 2223797 |
| AA493962 | 2223803 |
| AA494214 | 2224055 |
| AA494295 | 2224082 |
| AA494344 | 2224131 |
| AA494489 | 2224276 |
| AA494493 | 2224280 |
| AA495759 | 2229080 |
| AA495819 | 2229140 |
| AA495936 | 2229257 |
| AA495938 | 2229259 |
| AA495962 | 2229283 |
| AA496022 | 2229343 |
| AA496133 | 2229454 |
| AA496148 | 2229469 |
| AA496149 | 2229470 |
| AA496173 | 2229494 |
| AA496252 | 2229573 |
| AA496348 | 2229669 |
| AA496349 | 2229670 |
| AA496359 | 2229680 |
| AA496360 | 2229681 |
| AA496455 | 2229776 |
| AA496544 | 2229865 |
| AA496628 | 2229949 |
| AA496736 | 2230057 |
| AA496792 | 2230113 |
| AA496796 | 2230117 |
| AA496800 | 2230121 |
| AA496801 | 2230122 |
| AA496881 | 2230202 |
| AA496887 | 2230208 |
| AA496932 | 2230253 |
| AA496949 | 2230270 |
| AA496957 | 2230278 |
| AA496988 | 2230309 |
| AA496998 | 2230319 |
| AA497001 | 2230322 |
| AA497010 | 2230331 |
| AA497034 | 2230355 |
| AA497040 | 2230361 |
| AA497050 | 2230371 |
| AA497111 | 2230432 |
| AA497118 | 2230439 |
| AA497127 | 2230448 |
| AA501363 | 2236330 |
| AA501447 | 2236414 |
| AA501739 | 2236706 |
| AA501752 | 2236719 |
| AA501945 | 2236912 |
| AA501968 | 2236935 |
| AA502178 | 2237145 |
| AA502320 | 2237287 |
| AA502730 | 2237697 |
| AA502798 | 2237765 |
| AA502799 | 2237766 |
| AA502897 | 2237864 |
| AA502901 | 2237868 |
| AA502955 | 2237922 |
| AA502979 | 2237946 |
| AA503020 | 2237987 |
| AA503040 | 2238007 |
| AA503078 | 2238045 |
| AA503O96 | 2238063 |
| AA503115 | 2238082 |
| AA503132 | 2238099 |
| AA503182 | 2238149 |
| AA503215 | 2238182 |
| AA503224 | 2238191 |
| AA503265 | 2238232 |
| AA503330 | 2238297 |
| AA503611 | 2238578 |
| AA503718 | 2238685 |
| AA503719 | 2238686 |
| AA503731 | 2238698 |
| AA503930 | 2238897 |
| AA503943 | 2238910 |
| AA504120 | 2240280 |
| AA504132 | 2240292 |
| AA504134 | 2240294 |
| AA504173 | 2240333 |
| AA504201 | 2240361 |
| AA504202 | 2240362 |
| AA504238 | 2240398 |
| AA504239 | 2240399 |
| AA504246 | 2240406 |
| AA504262 | 2240422 |
| AA504265 | 2240425 |
| AA504272 | 2240432 |
| AA504284 | 2240444 |
| AA504322 | 2240482 |
| AA504334 | 2240494 |
| AA504340 | 2240500 |
| AA504343 | 2240503 |
| AA504354 | 2240514 |
| AA504490 | 2240650 |
| AA504492 | 2240652 |
| AA504505 | 2240665 |
| AA504507 | 2240667 |
| AA504514 | 2240674 |
| AA504536 | 2240696 |
| AA504560 | 2240720 |
| AA504617 | 2240777 |
| AA504622 | 2240782 |
| AA504625 | 2240785 |
| AA504631 | 2240791 |
| AA504703 | 2240863 |
| AA504728 | 2240888 |
| AA504772 | 2240932 |
| AA504783 | 2240943 |
| AA504784 | 2240944 |
| AA504834 | 2240994 |
| AA504838 | 2240998 |
| AA504842 | 2241002 |
| AA504844 | 2241004 |
| AA504845 | 2241005 |
| AA504953 | 2241113 |
| AA504969 | 2241129 |
| AA505045 | 2241205 |
| AA505051 | 2241211 |
| AA505063 | 2241223 |
| AA505073 | 2241233 |
| AA505076 | 2241236 |
| AA505082 | 2241242 |
| AA505117 | 2241277 |
| AA505124 | 2241284 |
| AA505141 | 2241301 |
| AA505150 | 2241310 |
| AA505162 | 2241322 |
| AA505198 | 2240236 |
| AA505327 | 2241464 |
| AA505400 | 2241537 |
| AA505423 | 2241560 |
| AA505523 | 2241660 |
| AA505588 | 2241705 |
| AA505594 | 2241731 |
| AA505625 | 2241762 |
| AA505816 | 2241953 |
| AA505953 | 2242090 |
| AA506099 | 2242236 |
| AA506299 | 2242539 |
| AA506304 | 2242544 |
| AA506323 | 2242563 |
| AA506419 | 2242659 |
| AA506459 | 2242699 |
| AA506607 | 2242754 |
| AA506729 | 2242876 |
| AA506923 | 2243362 |
| AA506953 | 2243392 |
| AA506967 | 2243406 |
| AA507041 | 2243480 |
| AA507065 | 2243504 |
| AA507084 | 2243523 |
| AA507201 | 2243640 |
| AA507217 | 2243656 |
| AA507306 | 2243745 |
| AA507314 | 2243753 |
| AA507442 | 2243881 |
| AA507472 | 2243911 |
| AA507505 | 2243944 |
| AA507511 | 2243950 |
| AA507595 | 2244034 |
| AA507777 | 2244216 |
| AA507791 | 2244230 |
| AA507792 | 2244231 |
| AA507804 | 2244243 |
| AA507878 | 2244317 |
| AA508507 | 2246010 |
| AA508552 | 2246055 |
| AA508579 | 2246082 |
| AA508588 | 2246091 |
| AA508804 | 2246307 |
| AA508810 | 2246313 |
| AA508861 | 2246364 |
| AA508889 | 2245830 |
| AA508898 | 2245839 |
| AA508902 | 2245843 |
| AA512974 | 2251397 |
| AA512975 | 2251398 |
| AA513052 | 2251464 |
| AA513056 | 2251468 |
| AA513177 | 2251589 |
| AA513556 | 2251968 |
| AA513597 | 2252009 |
| AA513688 | 2252100 |
| AA513940 | 2252361 |
| AA514042 | 2252463 |
| AA514302 | 2253810 |
| AA514324 | 2253832 |
| AA514474 | 2254074 |
| AA514490 | 2254090 |
| AA514775 | 2254375 |
| AA514933 | 2254533 |
| AA514938 | 2254538 |
| AA514991 | 2254591 |
| AA515109 | 2254709 |
| AA515132 | 2254732 |
| AA515143 | 2254743 |
| AA515564 | 2255164 |
| AA515576 | 2255176 |
| AA515857 | 2255457 |
| AA515908 | 2255508 |
| AA516441 | 2255965 |
| AA516531 | 2256055 |
| AA520979 | 2261522 |
| AA520999 | 2261542 |
| AA521013 | 2261556 |
| AA521016 | 2261559 |
| AA521083 | 2261626 |
| AA521108 | 2261651 |
| AA521140 | 2261683 |
| AA521142 | 2261685 |
| AA521163 | 2261706 |
| AA521213 | 2261756 |
| AA521232 | 2261775 |
| AA521243 | 2261786 |
| AA521247 | 2261790 |
| AA521292 | 2261835 |
| AA521327 | 2261870 |
| AA521337 | 2261880 |
| AA521351 | 2261894 |
| AA521384 | 2261927 |
| AA521385 | 2261928 |
| AA521394 | 2261937 |
| AA521409 | 2261952 |
| AA521411 | 2261954 |
| AA521414 | 2261957 |
| AA521416 | 2261959 |
| AA521431 | 2261974 |
| AA522473 | 2263185 |
| AA522481 | 2263193 |
| AA522492 | 2263204 |
| AA522549 | 2263261 |
| AA522552 | 2263264 |
| AA522638 | 2263350 |
| AA522664 | 2263376 |
| AA522686 | 2263398 |
| AA522746 | 2263458 |
| AA522849 | 2263561 |
| AA522850 | 2263562 |
| AA522856 | 2263568 |
| AA522867 | 2263579 |
| AA522936 | 2263648 |
| AA523006 | 2388773 |
| AA523252 | 2263964 |
| AA523354 | 2264066 |
| AA523464 | 2264176 |
| AA523484 | 2264196 |
| AA523498 | 2264210 |
| AA523503 | 2264215 |
| AA523652 | 2264580 |
| AA523671 | 2264599 |
| AA523677 | 2264605 |
| AA523708 | 2264636 |
| AA523751 | 2264679 |
| AA523902 | 2264830 |
| AA523938 | 2264866 |
| AA523982 | 2264910 |
| AA524023 | 2264951 |
| AA524064 | 2264992 |
| AA524258 | 2265186 |
| AA524285 | 2265213 |
| AA524462 | 2265390 |
| AA524485 | 2265413 |
| AA524528 | 2265456 |
| AA524651 | 2265579 |
| AA524672 | 2265600 |
| AA524778 | 2265706 |
| AA524812 | 2265740 |
| AA524893 | 2265821 |
| AA525032 | 2265960 |
| AA525269 | 2266197 |
| AA525386 | 2266314 |
| AA525419 | 2266347 |
| AA525487 | 2264442 |
| AA525497 | 2264452 |
| AA525801 | 2267870 |
| AA525958 | 2268027 |
| AA525960 | 2268029 |
| AA526097 | 2268166 |
| AA526151 | 2268220 |
| AA526187 | 2268256 |
| AA526214 | 2268283 |
| AA526243 | 2268312 |
| AA526244 | 2268313 |
| AA526283 | 2268352 |
| AA526368 | 2268437 |
| AA526369 | 2268438 |
| AA526403 | 2268472 |
| AA526430 | 2268499 |
| AA526472 | 2268541 |
| AA526515 | 2268584 |
| AA526591 | 2268660 |
| AA526834 | 2268903 |
| AA526869 | 2268938 |
| AA526886 | 2268955 |
| AA526893 | 2268962 |
| AA526894 | 2268963 |
| AA527116 | 2269185 |
| AA527187 | 2269256 |
| AA527272 | 2269341 |
| AA527326 | 2269395 |
| AA527342 | 2269411 |
| AA527570 | 2269639 |
| AA527667 | 2269736 |
| AA527730 | 2269799 |
| AA527737 | 2269806 |
| AA527805 | 2269874 |
| AA528023 | 2270092 |
| AA528104 | 2270173 |
| AA528123 | 2270192 |
| AA528202 | 2270271 |
| AA528204 | 2270273 |
| AA531044 | 2273750 |
| AA531068 | 2273774 |
| AA531198 | 2273904 |
| AA531221 | 2273927 |
| AA531227 | 2273933 |
| AA531249 | 2273955 |
| AA531255 | 2273961 |
| AA531312 | 2274018 |
| AA531386 | 2274092 |
| AA531487 | 2274193 |
| AA531509 | 2274215 |
| AA531561 | 2274267 |
| AA531563 | 2274269 |
| AA531582 | 2274288 |
| AA531602 | 2274308 |
| AA531606 | 2274312 |
| AA532377 | 2276631 |
| AA532398 | 2276652 |
| AA532461 | 2276715 |
| AA532663 | 2276917 |
| AA532852 | 2278428 |
| AA532883 | 2276979 |
| AA532934 | 2277030 |
| AA533001 | 2277097 |
| AA533057 | 2277153 |
| AA533115 | 2277211 |
| AA533227 | 2277323 |
| AA533372 | 2277468 |
| AA533386 | 2277482 |
| AA533494 | 2277590 |
| AA533553 | 2277649 |
| AA533575 | 2278478 |
| AA533648 | 2277664 |
| AA533709 | 2277725 |
| AA533716 | 2277732 |
| AA533772 | 2277788 |
| AA533785 | 2277801 |
| AA533940 | 2277956 |
| AA533963 | 2277979 |
| AA534171 | 2278187 |
| AA534173 | 2278189 |
| AA534235 | 2278251 |
| AA534246 | 2278262 |
| AA534281 | 2278297 |
| AA534298 | 2278551 |
| AA534395 | 2278648 |
| AA534418 | 2278671 |
| AA534419 | 2278672 |
| AA534435 | 2278688 |
| AA534505 | 2278758 |
| AA534540 | 2278793 |
| AA534543 | 2278796 |
| AA534644 | 2278897 |
| AA534778 | 2279031 |
| AA534827 | 2279080 |
| AA534873 | 2279126 |
| AA534933 | 2279186 |
| AA535185 | 2279438 |
| AA535218 | 2279471 |
| AA535377 | 2279630 |
| AA535384 | 2279637 |
| AA535610 | 2279863 |
| AA535695 | 2279948 |
| AA535755 | 2280008 |
| AA535837 | 2280090 |
| AA536036 | 2280289 |
| AA536129 | 2280382 |
| AA536175 | 2280428 |
| AA541515 | 2287949 |
| AA541528 | 2287962 |
| AA541537 | 2287971 |
| AA541587 | 2288021 |
| AA541677 | 2288111 |
| AA541678 | 2288112 |
| AA541703 | 2288137 |
| AA541808 | 2288242 |
| AA541813 | 2288247 |
| AA542906 | 2291386 |
| AA542981 | 2291461 |
| AA543102 | 2291582 |
| AA545726 | 2307096 |
| AA545727 | 2307097 |
| AA548098 | 2318380 |
| AA548124 | 2318406 |
| AA548129 | 2318411 |
| AA548238 | 2318520 |
| AA548244 | 2318526 |
| AA548308 | 2318590 |
| AA548465 | 2318747 |
| AA548472 | 2318754 |
| AA548600 | 2318882 |
| AA548722 | 2319004 |
| AA548764 | 2319046 |
| AA550927 | 2321179 |
| AA551005 | 2321257 |
| AA551065 | 2321317 |
| AA551146 | 2321398 |
| AA551189 | 2321441 |
| AA551252 | 2321504 |
| AA551464 | 2321716 |
| AA551773 | 2322025 |
| AA551894 | 2322146 |
| AA552154 | 2322406 |
| AA552253 | 2322505 |
| AA552321 | 2322573 |
| AA552410 | 2322664 |
| AA552570 | 2322824 |
| AA552715 | 2322969 |
| AA552842 | 2323096 |
| AA553726 | 2324265 |
| AA553789 | 2324328 |
| AA553826 | 2324365 |
| AA554010 | 2324549 |
| AA554018 | 2324557 |
| AA554326 | 2324865 |
| AA554358 | 2324897 |
| AA554598 | 2325137 |
| AA554661 | 2325200 |
| AA554718 | 2325257 |
| AA554735 | 2325274 |
| AA555115 | 2325654 |
| AA555160 | 2325699 |
| AA555194 | 2325733 |
| AA555227 | 2325766 |
| AA557174 | 2327651 |
| AA557260 | 2327737 |
| AA557321 | 2327798 |
| AA557408 | 2327885 |
| AA557683 | 2328160 |
| AA557888 | 2328365 |
| AA558280 | 2328757 |
| AA558281 | 2328758 |
| AA558547 | 2329024 |
| AA558611 | 2329088 |
| AA558636 | 2329113 |
| AA558755 | 2329232 |
| AA558976 | 2329743 |
| AA559055 | 2329822 |
| AA559906 | 2331397 |
| AA563986 | 2335625 |
| AA564005 | 2335644 |
| AA564017 | 2335656 |
| AA564067 | 2335706 |
| AA564119 | 2335758 |
| AA564238 | 2335877 |
| AA564277 | 2335916 |
| AA564484 | 2336123 |
| AA564505 | 2336144 |
| AA564513 | 2336152 |
| AA564843 | 2336482 |
| AA565044 | 2336683 |
| AA565214 | 2336853 |
| AA565272 | 2336911 |
| AA565334 | 2336973 |
| AA565420 | 2337059 |
| AA565448 | 2337087 |
| AA565882 | 2337521 |
| AA565996 | 2337635 |
| AA568138 | 2341192 |
| AA568207 | 2341261 |
| AA568406 | 2341460 |
| AA568488 | 2341542 |
| AA568518 | 2341572 |
| AA568538 | 2341592 |
| AA568990 | 2342044 |
| AA569130 | 2342184 |
| AA569247 | 2342301 |
| AA569344 | 2342398 |
| AA569354 | 2342408 |
| AA569434 | 2343414 |
| AA569439 | 2343419 |
| AA569482 | 2343462 |
| AA569493 | 2343473 |
| AA569765 | 2343745 |
| AA569809 | 2343789 |
| AA569813 | 2343793 |
| AA570171 | 2344151 |
| AA570182 | 2344162 |
| AA570519 | 2344499 |
| AA570665 | 2344645 |
| AA570748 | 2343339 |
| AA572808 | 2347336 |
| AA572895 | 2347423 |
| AA572938 | 2347466 |
| AA572950 | 2347478 |
| AA573187 | 2347715 |
| AA573368 | 2347896 |
| AA573557 | 2348085 |
| AA573559 | 2348087 |
| AA573575 | 2348103 |
| AA573646 | 2360882 |
| AA573737 | 2348252 |
| AA573742 | 2348257 |
| AA573762 | 2348277 |
| AA573787 | 2348302 |
| AA573811 | 2348326 |
| AA573817 | 2348332 |
| AA573824 | 2348339 |
| AA573910 | 2348425 |
| AA573974 | 2348489 |
| AA573975 | 2348490 |
| AA574223 | 2348738 |
| AA574227 | 2348742 |
| AA574248 | 2348763 |
| AA574288 | 2348803 |
| AA574397 | 2348912 |
| AA574436 | 2348951 |
| AA575882 | 2350397 |
| AA575898 | 2350413 |
| AA575933 | 2350448 |
| AA575935 | 2350450 |
| AA576243 | 2350758 |
| AA576252 | 2350767 |
| AA576404 | 2353904 |
| AA576407 | 2353907 |
| AA576425 | 2353925 |
| AA576669 | 2354143 |
| AA577326 | 2354800 |
| AA577463 | 2354937 |
| AA577490 | 2354964 |
| AA577553 | 2355027 |
| AA577557 | 2355031 |
| AA577585 | 2355059 |
| AA577598 | 2355072 |
| AA577672 | 2355856 |
| AA577800 | 2355984 |
| AA578009 | 2356193 |
| AA578209 | 2356393 |
| AA578265 | 2356449 |
| AA578295 | 2356479 |
| AA578511 | 2356695 |
| AA578577 | 2356761 |
| AA578646 | 2356830 |
| AA578773 | 2356957 |
| AA578881 | 2357065 |
| AA578904 | 2357088 |
| AA578976 | 2357160 |
| AA579026 | 2357210 |
| AA579039 | 2357223 |
| AA579160 | 2357344 |
| AA579384 | 2357568 |
| AA579412 | 2357596 |
| AA579486 | 2357670 |
| AA579596 | 2357780 |
| AA579617 | 2357801 |
| AA579835 | 2355162 |
| AA579904 | 2355231 |
| AA579998 | 2355325 |
| AA580097 | 2355424 |
| AA580279 | 2355606 |
| AA580399 | 2355726 |
| AA580417 | 2355744 |
| AA580771 | 2358543 |
| AA580829 | 2358601 |
| AA580900 | 2358672 |
| AA581117 | 2358889 |
| AA581222 | 2358994 |
| AA581523 | 2359295 |
| AA581716 | 2359488 |
| AA581829 | 2360507 |
| AA581846 | 2360524 |
| AA581892 | 2360570 |
| AA581956 | 2360634 |
| AA582252 | 2359612 |
| AA582489 | 2359349 |
| AA582591 | 2359951 |
| AA582596 | 2359956 |
| AA582728 | 2360088 |
| AA582805 | 2360165 |
| AA582930 | 2360290 |
| AA583350 | 2367959 |
| AA583353 | 2367962 |
| AA583431 | 2368040 |
| AA583449 | 2368058 |
| AA583461 | 2368070 |
| AA583567 | 2368176 |
| AA583574 | 2368183 |
| AA583773 | 2368382 |
| AA583801 | 2368410 |
| AA583899 | 2368508 |
| AA583997 | 2368606 |
| AA584026 | 2368635 |
| AA584428 | 2369037 |
| AA585110 | 2384998 |
| AA585278 | 2385166 |
| AA585430 | 2385318 |
| AA586522 | 2398517 |
| AA586744 | 2397558 |
| AA586969 | 2397783 |
| AA587020 | 2397834 |
| AA587140 | 2397954 |
| AA587226 | 2398040 |
| AA587243 | 2398057 |
| AA587348 | 2398162 |
| AA587459 | 2398273 |
| AA587468 | 2398282 |
| AA587630 | 2401805 |
| AA587806 | 2401981 |
| AA588023 | 2402198 |
| AA588083 | 2402258 |
| AA588358 | 2401533 |
| AA588474 | 2401649 |
| AA588685 | 2402416 |
| AA588754 | 2402485 |
| AA588801 | 2402532 |
| AA588828 | 2402559 |
| AA588848 | 2401278 |
| AA588882 | 2401312 |
| AA592908 | 2408670 |
| AA592947 | 2408709 |
| AA593159 | 2408921 |
| AA593385 | 2409147 |
| AA593668 | 2408346 |
| AA593879 | 2408557 |
| AA594131 | 2409481 |
| AA594282 | 2409632 |
| AA594540 | 2409890 |
| AA594681 | 2410031 |
| AA594682 | 2410032 |
| AA594780 | 2410130 |
| AA594950 | 2410300 |
| AA594976 | 2410326 |
| AA595047 | 2410397 |
| AA595100 | 2410450 |
| AA595193 | 2410543 |
| AA595339 | 2410689 |
| AA595471 | 2410821 |
| AA595581 | 2410931 |
| AA595582 | 2410932 |
| AA598470 | 2432053 |
| AA598483 | 2432066 |
| AA598487 | 2432070 |
| AA598517 | 2432100 |
| AA598520 | 2432103 |
| AA598531 | 2432114 |
| AA598538 | 2432121 |
| AA598561 | 2432144 |
| AA598567 | 2432150 |
| AA598572 | 2432155 |
| AA598595 | 2432178 |
| AA598597 | 2432180 |
| AA598598 | 2432181 |
| AA598601 | 2432184 |
| AA598621 | 2432204 |
| AA598625 | 2432208 |
| AA598652 | 2432235 |
| AA598659 | 2432242 |
| AA598668 | 2432251 |
| AA598675 | 2432258 |
| AA598758 | 2432430 |
| AA598775 | 2432447 |
| AA598779 | 2432451 |
| AA598787 | 2432459 |
| AA598795 | 2432467 |
| AA598796 | 2432468 |
| AA598797 | 2432469 |
| AA598803 | 2432475 |
| AA598808 | 2432480 |
| AA598809 | 2432481 |
| AA598815 | 2432487 |
| AA598817 | 2432489 |
| AA598828 | 2432500 |
| AA598849 | 2432521 |
| AA598868 | 2432540 |
| AA598874 | 2432546 |
| AA598943 | 2432615 |
| AA598956 | 2432628 |
| AA598965 | 2432264 |
| AA598974 | 2432273 |
| AA598975 | 2432274 |
| AA598982 | 2432022 |
| AA598983 | 2432023 |
| AA598987 | 2432027 |
| AA598996 | 2432036 |
| AA599064 | 2432689 |
| AA599078 | 2432703 |
| AA599092 | 2432717 |
| AA599094 | 2432719 |
| AA599099 | 2432724 |
| AA599102 | 2432727 |
| AA599107 | 2432732 |
| AA599120 | 2432745 |
| AA599178 | 2432803 |
| AA599229 | 2432854 |
| AA599324 | 2432949 |
| AA599416 | 2433041 |
| AA599424 | 2433049 |
| AA599533 | 2433158 |
| AA599534 | 2433159 |
| AA599650 | 2433275 |
| AA599669 | 2433294 |
| AA599711 | 2433336 |
| AA599717 | 2433342 |
| AA599741 | 2433366 |
| AA599864 | 2433489 |
| AA600060 | 2433685 |
| AA600257 | 2433882 |
| AA600974 | 2434599 |
| AA600998 | 2434623 |
| AA601289 | 2434914 |
| AA602361 | 2436339 |
| AA602430 | 2435926 |
| AA602472 | 2436406 |
| AA602522 | 2436456 |
| AA602634 | 2436568 |
| AA602662 | 2436596 |
| AA602675 | 2436609 |
| AA602758 | 2436692 |
| AA602773 | 2436707 |
| AA602794 | 2436728 |
| AA602957 | 2436009 |
| AA602961 | 2436013 |
| AA602979 | 2436840 |
| AA603041 | 2436902 |
| AA603135 | 2436996 |
| AA603158 | 2437019 |
| AA603265 | 2437126 |
| AA603334 | 2437195 |
| AA603572 | 2437433 |
| AA603834 | 2437695 |
| AA603861 | 2437722 |
| AA604229 | 2445138 |
| AA604474 | 2445383 |
| AA604497 | 2444773 |
| AA604619 | 2445483 |
| AA604815 | 2445679 |
| AA605077 | 2444896 |
| AA608514 | 2456942 |
| AA608546 | 2456974 |
| AA608548 | 2456976 |
| AA608567 | 2456995 |
| AA608568 | 2456996 |
| AA608575 | 2457003 |
| AA608583 | 2457011 |
| AA608636 | 2457064 |
| AA608679 | 2457107 |
| AA608730 | 2457158 |
| AA608749 | 2457177 |
| AA608752 | 2457180 |
| AA608770 | 2457198 |
| AA608832 | 2457260 |
| AA608856 | 2457284 |
| AA608893 | 2457321 |
| AA608952 | 2457380 |
| AA608967 | 2457395 |
| AA608974 | 2457402 |
| AA609002 | 2457430 |
| AA609004 | 2457432 |
| AA609005 | 2457433 |
| AA609049 | 2457477 |
| AA609054 | 2457482 |
| AA609056 | 2457484 |
| AA609088 | 2457516 |
| AA609094 | 2457522 |
| AA609106 | 2457534 |
| AA609135 | 2457563 |
| AA609138 | 2457566 |
| AA609189 | 2457617 |
| AA609218 | 2457646 |
| AA609284 | 2457712 |
| AA609300 | 2457728 |
| AA609304 | 2457732 |
| AA609323 | 2457751 |
| AA609334 | 2457762 |
| AA609365 | 2457793 |
| AA609368 | 2457796 |
| AA609384 | 2457812 |
| AA609385 | 2457813 |
| AA609392 | 2457820 |
| AA609394 | 2457822 |
| AA609415 | 2457843 |
| AA609422 | 2457850 |
| AA609430 | 2457858 |
| AA609436 | 2457864 |
| AA609456 | 2457884 |
| AA609471 | 2457899 |
| AA609474 | 2457902 |
| AA609485 | 2457913 |
| AA609556 | 2457984 |
| AA609566 | 2457994 |
| AA609584 | 2458012 |
| AA609594 | 2458022 |
| AA609641 | 2458069 |
| AA609686 | 2458114 |
| AA609723 | 2458151 |
| AA609731 | 2458159 |
| AA609744 | 2458172 |
| AA609749 | 2458177 |
| AA609887 | 2458315 |
| AA609891 | 2458319 |
| AA609914 | 2458342 |
| AA609982 | 2458410 |
| AA609983 | 2458411 |
| AA610052 | 2458480 |
| AA610071 | 2458499 |
| AA610352 | 2458780 |
| AA610444 | 2458872 |
| AA610476 | 2458904 |
| AA610581 | 2459009 |
| AA610734 | 2459162 |
| AA612573 | 2464770 |
| AA612666 | 2463704 |
| AA612829 | 2463867 |
| AA612864 | 2463902 |
| AA612874 | 2463912 |
| AA613314 | 2464352 |
| AA613587 | 2464625 |
| AA613772 | 2466463 |
| AA613891 | 2466025 |
| AA613908 | 2466042 |
| AA613916 | 2466050 |
| AA613933 | 2466067 |
| AA613935 | 2466069 |
| AA614105 | 2466239 |
| AA614274 | 2466408 |
| AA614350 | 2466546 |
| AA614525 | 2466721 |
| AA617696 | 2504901 |
| AA617722 | 2504927 |
| AA618183 | 2505388 |
| AA618277 | 2505482 |
| AA618435 | 2505640 |
| AA618484 | 2505689 |
| AA618486 | 2505691 |
| AA618601 | 2505806 |
| AA620301 | 2524240 |
| AA620334 | 2524273 |
| AA620396 | 2524335 |
| AA620404 | 2524343 |
| AA620421 | 2524360 |
| AA620437 | 2524376 |
| AA620446 | 2524385 |
| AA620458 | 2524397 |
| AA620463 | 2524402 |
| AA620466 | 2524405 |
| AA620472 | 2524411 |
| AA620485 | 2524424 |
| AA620528 | 2524467 |
| AA620553 | 2524492 |
| AA620556 | 2524495 |
| AA620565 | 2524504 |
| AA620591 | 2524530 |
| AA620607 | 2524546 |
| AA620608 | 2524547 |
| AA620611 | 2524550 |
| AA620614 | 2524553 |
| AA620642 | 2524581 |
| AA620697 | 2524636 |
| AA620715 | 2524654 |
| AA620747 | 2524686 |
| AA620750 | 2524689 |
| AA620757 | 2524696 |
| AA620760 | 2524699 |
| AA620783 | 2524722 |
| AA620927 | 2524866 |
| AA620995 | 2524934 |
| AA621001 | 2524940 |
| AA621019 | 2524958 |
| AA621031 | 2524970 |
| AA621073 | 2525012 |
| AA621165 | 2525104 |
| AA621218 | 2525157 |
| AA621310 | 2525249 |
| AA621324 | 2525263 |
| AA621332 | 2525271 |
| AA621342 | 2525281 |
| AA621355 | 2525294 |
| AA621363 | 2525302 |
| AA621405 | 2525344 |
| AA621457 | 2525396 |
| AA621478 | 2525417 |
| AA621730 | 2525669 |
| AA621750 | 2524178 |
| AA621761 | 2524189 |
| AA621820 | 2525696 |
| AA622344 | 2526220 |
| AA622418 | 2526294 |
| AA622482 | 2526358 |
| AA622765 | 2526641 |
| AA625141 | 2537526 |
| AA625145 | 2537530 |
| AA625222 | 2537607 |
| AA625228 | 2537613 |
| AA625270 | 2537655 |
| AA625415 | 2537802 |
| AA625467 | 2537854 |
| AA625485 | 2537872 |
| AA625538 | 2537925 |
| AA625558 | 2537945 |
| AA625567 | 2537954 |
| AA625574 | 2537961 |
| AA625628 | 2538015 |
| AA625634 | 2538021 |
| AA625641 | 2538028 |
| AA625662 | 2538049 |
| AA625666 | 2538053 |
| AA625675 | 2538062 |
| AA625682 | 2538069 |
| AA625764 | 2538151 |
| AA625784 | 2538171 |
| AA625788 | 2538175 |
| AA625791 | 2538178 |
| AA625812 | 2538199 |
| AA625833 | 2538220 |
| AA625890 | 2538277 |
| AA625907 | 2538294 |
| AA625951 | 2538338 |
| AA625979 | 2538366 |
| AA625990 | 2538377 |
| AA625995 | 2538382 |
| AA626011 | 2538398 |
| AA626028 | 2538415 |
| AA626146 | 2538533 |
| AA626152 | 2538539 |
| AA626161 | 2538548 |
| AA626167 | 2538554 |
| AA626242 | 2538629 |
| AA626247 | 2538634 |
| AA626248 | 2538635 |
| AA626274 | 2538661 |
| AA626275 | 2538662 |
| AA626315 | 2538702 |
| AA626365 | 2538752 |
| AA626439 | 2538826 |
| AA626509 | 2538896 |
| AA626511 | 2538898 |
| AA626698 | 2539085 |
| AA626784 | 2539171 |
| AA626788 | 2539175 |
| AA626847 | 2539234 |
| AA626864 | 2539251 |
| AA627073 | 2540117 |
| AA627785 | 2539880 |
| AA627855 | 2540421 |
| AA627939 | 2539938 |
| AA628113 | 2540500 |
| AA628132 | 2540519 |
| AA628146 | 2540533 |
| AA628190 | 2540577 |
| AA628194 | 2540581 |
| AA628409 | 2540796 |
| AA628410 | 2540797 |
| AA628417 | 2540804 |
| AA628526 | 2540913 |
| AA628530 | 2540917 |
| AA628867 | 2541254 |
| AA629020 | 2541407 |
| AA629027 | 2541414 |
| AA629033 | 2541420 |
| AA629039 | 2541426 |
| AA629117 | 2541504 |
| AA629251 | 2541638 |
| AA629262 | 2541649 |
| AA629269 | 2541656 |
| AA629324 | 2541711 |
| AA629325 | 2541712 |
| AA629355 | 2541742 |
| AA629357 | 2541744 |
| AA629524 | 2552135 |
| AA629528 | 2552139 |
| AA629534 | 2552145 |
| AA629554 | 2552165 |
| AA629581 | 2552192 |
| AA629591 | 2552202 |
| AA629608 | 2552219 |
| AA629707 | 2552318 |
| AA629719 | 2552330 |
| AA629771 | 2552382 |
| AA629801 | 2552412 |
| AA629804 | 2552415 |
| AA629808 | 2552419 |
| AA629838 | 2552449 |
| AA629849 | 2552460 |
| AA629903 | 2552514 |
| AA629910 | 2552521 |
| AA629911 | 2552522 |
| AA629991 | 2552602 |
| AA629999 | 2552610 |
| AA630006 | 2552617 |
| AA630016 | 2552627 |
| AA630084 | 2552695 |
| AA630104 | 2552715 |
| AA630286 | 2552897 |
| AA630298 | 2552909 |
| AA630303 | 2552914 |
| AA630325 | 2552936 |
| AA630328 | 2552939 |
| AA630346 | 2552957 |
| AA630373 | 2552984 |
| AA630374 | 2552985 |
| AA630376 | 2552987 |
| AA630382 | 2552993 |
| AA630445 | 2553056 |
| AA630545 | 2553156 |
| AA630584 | 2553195 |
| AA630620 | 2553231 |
| AA630771 | 2553382 |
| AA630800 | 2553411 |
| AA631009 | 2553620 |
| AA631152 | 2553763 |
| AA631176 | 2553787 |
| AA631178 | 2553789 |
| AA631206 | 2553817 |
| AA631303 | 2553914 |
| AA631419 | 2554030 |
| AA631437 | 2554048 |
| AA631446 | 2554057 |
| AA631460 | 2554071 |
| AA631608 | 2554219 |
| AA631868 | 2554479 |
| AA631903 | 2554514 |
| AA632154 | 2555568 |
| AA632291 | 2555705 |
| AA632363 | 2555777 |
| AA632427 | 2555841 |
| AA632742 | 2556156 |
| AA632954 | 2556368 |
| AA633347 | 2555207 |
| AA633399 | 2555259 |
| AA633439 | 2555299 |
| AA633577 | 2556791 |
| AA633658 | 2556872 |
| AA633768 | 2556982 |
| AA633794 | 2557008 |
| AA633811 | 2557025 |
| AA633835 | 2557049 |
| AA633846 | 2557060 |
| AA633866 | 2557080 |
| AA633909 | 2557123 |
| AA633957 | 2557171 |
| AA633993 | 2557207 |
| AA634008 | 2557222 |
| AA634039 | 2557253 |
| AA634120 | 2557334 |
| AA634164 | 2557378 |
| AA634196 | 2557410 |
| AA634213 | 2557427 |
| AA634216 | 2557430 |
| AA634291 | 2557505 |
| AA634360 | 2557574 |
| AA634379 | 2557593 |
| AA634408 | 2557622 |
| AA634424 | 2557638 |
| AA634431 | 2557645 |
| AA634479 | 2557693 |
| AA634482 | 2557696 |
| AA634772 | 2557986 |
| AA634799 | 2558013 |
| AA635024 | 2558238 |
| AA635186 | 2558400 |
| AA635288 | 2619093 |
| AA635393 | 2559235 |
| AA635590 | 2559432 |
| AA638989 | 2562768 |
| AA639059 | 2562838 |
| AA639174 | 2562953 |
| AA639317 | 2563096 |
| AA639786 | 2563565 |
| AA639791 | 2563570 |
| AA639902 | 2563681 |
| AA639964 | 2563743 |
| AA639972 | 2563751 |
| AA639999 | 2563778 |
| AA640038 | 2563817 |
| AA640105 | 2563884 |
| AA640133 | 2563912 |
| AA640141 | 2563920 |
| AA640280 | 2565530 |
| AA640458 | 2565708 |
| AA640474 | 2565724 |
| AA640574 | 2565824 |
| AA640687 | 2565937 |
| AA640716 | 2565966 |
| AA640889 | 2566139 |
| AA640928 | 2566178 |
| AA641074 | 2566324 |
| AA641092 | 2566342 |
| AA641195 | 2566445 |
| AA641520 | 2566770 |
| AA641585 | 2565459 |
| AA641601 | 2565475 |
| AA641623 | 2566841 |
| AA641636 | 2566854 |
| AA641769 | 2566987 |
| AA641810 | 2589258 |
| AA642383 | 2567601 |
| AA642471 | 2567689 |
| AA642495 | 2567713 |
| AA643506 | 2568724 |
| AA643685 | 2568903 |
| AA643720 | 2568938 |
| AA644080 | 2569298 |
| AA644092 | 2569310 |
| AA644099 | 2569317 |
| AA644211 | 2569429 |
| AA644237 | 2569455 |
| AA644334 | 2569552 |
| AA644345 | 2569563 |
| AA644495 | 2569713 |
| AA644550 | 2569768 |
| AA644563 | 2569781 |
| AA644624 | 2569842 |
| AA644678 | 2569896 |
| AA644700 | 2569918 |
| AA648194 | 2574623 |
| AA648209 | 2574638 |
| AA648318 | 2574747 |
| AA648508 | 2574937 |
| AA648757 | 2575186 |
| AA648989 | 2575418 |
| AA649012 | 2575441 |
| AA649107 | 2575536 |
| AA649193 | 2575622 |
| AA649298 | 2575727 |
| AA649627 | 2576955 |
| AA650114 | 2577442 |
| AA650230 | 2577558 |
| AA650241 | 2577569 |
| AA651712 | 2583364 |
| AA652167 | 2583819 |
| AA652206 | 2583858 |
| AA652478 | 2584130 |
| AA652600 | 2584252 |
| AA652667 | 2584319 |
| AA652846 | 2584498 |
| AA652925 | 2584577 |
| AA653353 | 2589524 |
| AA653508 | 2589679 |
| AA653601 | 2589772 |
| AA654527 | 2590681 |
| AA654557 | 2590711 |
| AA654621 | 2590775 |
| AA654650 | 2590804 |
| AA654675 | 2590829 |
| AA654709 | 2590863 |
| AA654875 | 2591029 |
| AA654995 | 2591149 |
| AA657411 | 2593565 |
| AA657463 | 2593617 |
| AA657488 | 2593642 |
| AA657830 | 2593984 |
| AA658207 | 2594361 |
| AA658393 | 2594547 |
| AA659142 | 2595296 |
| AA659179 | 2595333 |
| AA659388 | 2595542 |
| AA659391 | 2595545 |
| AA659693 | 2595847 |
| AA659719 | 2595873 |
| AA659725 | 2595879 |
| AA659764 | 2595918 |
| AA661464 | 2615555 |
| AA661927 | 2616018 |
| AA662111 | 2616202 |
| AA662369 | 2616460 |
| AA662406 | 2616497 |
| AA662658 | 2616649 |
| AA662702 | 2616693 |
| AA662733 | 2616724 |
| AA662822 | 2616813 |
| AA663058 | 2617049 |
| AA663092 | 2617083 |
| AA663123 | 2617114 |
| AA663160 | 2617151 |
| AA663192 | 2617183 |
| AA663310 | 2617301 |
| AA663453 | 2617444 |
| AA663556 | 2617547 |
| AA663592 | 2617583 |
| AA663826 | 2617817 |
| AA663960 | 2617951 |
| AA663981 | 2617972 |
| AA663983 | 2617974 |
| AA663995 | 2617986 |
| AA664007 | 2617998 |
| AA664067 | 2618058 |
| AA664101 | 2618092 |
| AA664104 | 2618095 |
| AA664105 | 2618096 |
| AA664155 | 2618146 |
| AA664389 | 2618380 |
| AA664406 | 2618397 |
| AA664472 | 2618463 |
| AA664480 | 2618471 |
| AA664482 | 2618473 |
| AA664567 | 2619180 |
| AA664798 | 2619411 |
| AA665209 | 2880095 |
| AA665507 | 2620120 |
| AA665901 | 2620514 |
| AA666087 | 2620700 |
| AA666348 | 2620961 |
| AA666366 | 2620979 |
| AA666405 | 2621018 |
| AA666418 | 2621031 |
| AA668178 | 2629677 |
| AA668224 | 2629723 |
| AA668230 | 2629729 |
| AA668470 | 2629969 |
| AA668531 | 2630030 |
| AA668692 | 2630191 |
| AA668897 | 2630396 |
| AA668951 | 2630450 |
| AA668959 | 2630458 |
| AA669124 | 2630623 |
| AA669183 | 2630682 |
| AA669218 | 2630717 |
| AA669222 | 2630721 |
| AA669300 | 2630799 |
| AA669314 | 2630813 |
| AA669341 | 2630840 |
| AA669438 | 2630937 |
| AA669443 | 2630942 |
| AA669451 | 2630950 |
| AA669452 | 2630951 |
| AA669484 | 2630983 |
| AA669544 | 2631043 |
| AA669750 | 2631249 |
| AA669823 | 2631322 |
| AA669976 | 2631475 |
| AA670123 | 2631622 |
| AA670200 | 2631699 |
| AA670205 | 2631704 |
| AA670280 | 2631779 |
| AA670286 | 2631785 |
| AA670296 | 2631795 |
| AA670330 | 2631829 |
| AA670353 | 2631852 |
| AA670356 | 2631855 |
| AA670359 | 2631858 |
| AA670390 | 2631889 |
| AA670408 | 2631907 |
| AA670411 | 2631910 |
| AA670429 | 2631928 |
| AA675891 | 2775238 |
| AA675892 | 2775239 |
| AA676225 | 2656747 |
| AA676265 | 2656787 |
| AA676296 | 2656818 |
| AA676422 | 2656944 |
| AA676460 | 2656982 |
| AA676470 | 2656992 |
| AA676515 | 2657037 |
| AA676590 | 2657112 |
| AA676598 | 2657120 |
| AA676604 | 2657126 |
| AA676740 | 2657262 |
| AA676765 | 2657287 |
| AA676840 | 2657362 |
| AA676865 | 2657387 |
| AA676899 | 2657421 |
| AA676920 | 2657442 |
| AA676948 | 2657470 |
| AA676961 | 2657483 |
| AA676998 | 2657520 |
| AA677023 | 2657545 |
| AA677025 | 2657547 |
| AA677050 | 2657572 |
| AA677165 | 2657687 |
| AA677185 | 2657707 |
| AA677200 | 2657722 |
| AA677206 | 2657728 |
| AA677282 | 2657804 |
| AA677287 | 2657809 |
| AA677295 | 2657817 |
| AA677300 | 2657822 |
| AA677337 | 2657859 |
| AA677388 | 2657910 |
| AA677397 | 2657919 |
| AA677403 | 2657925 |
| AA677457 | 2657979 |
| AA677461 | 2657983 |
| AA677499 | 2658021 |
| AA677522 | 2658044 |
| AA677531 | 2658053 |
| AA677534 | 2658056 |
| AA677550 | 2658072 |
| AA677561 | 2658083 |
| AA677572 | 2658094 |
| AA677574 | 2658096 |
| AA677575 | 2658097 |
| AA677629 | 2658151 |
| AA677643 | 2658165 |
| AA677655 | 2658177 |
| AA677706 | 2658228 |
| AA677984 | 2658506 |
| AA678021 | 2658543 |
| AA678065 | 2658587 |
| AA678072 | 2658594 |
| AA678087 | 2658609 |
| AA678135 | 2658657 |
| AA678164 | 2658686 |
| AA678203 | 2658725 |
| AA678295 | 2658817 |
| AA678348 | 2658870 |
| AA678361 | 2658883 |
| AA678414 | 2658936 |
| AA678873 | 2659395 |
| AA678975 | 2659497 |
| AA679068 | 2659590 |
| AA679116 | 2659638 |
| AA679180 | 2659702 |
| AA679304 | 2659826 |
| AA679314 | 2659836 |
| AA679341 | 2659863 |
| AA679352 | 2659874 |
| AA679414 | 2659936 |
| AA679434 | 2659956 |
| AA679547 | 2660069 |
| AA679569 | 2660091 |
| AA679791 | 2660313 |
| AA679907 | 2656374 |
| AA679935 | 2656402 |
| AA680052 | 2656519 |
| AA680070 | 2656537 |
| AA680136 | 2656603 |
| AA680186 | 2656653 |
| AA680244 | 2656212 |
| AA680351 | 2656658 |
| AA682233 | 2669365 |
| AA682244 | 2669376 |
| AA682252 | 2669384 |
| AA682274 | 2669591 |
| AA682283 | 2669600 |
| AA682293 | 2669610 |
| AA682321 | 2669638 |
| AA682386 | 2669667 |
| AA682399 | 2669680 |
| AA682405 | 2669686 |
| AA682423 | 2669704 |
| AA682425 | 2669706 |
| AA682439 | 2669720 |
| AA682452 | 2669733 |
| AA682533 | 2669814 |
| AA682545 | 2669826 |
| AA682549 | 2669830 |
| AA682565 | 2669846 |
| AA682627 | 2669908 |
| AA682631 | 2669912 |
| AA682642 | 2669923 |
| AA682819 | 2669502 |
| AA682838 | 2669521 |
| AA682851 | 2669534 |
| AA682905 | 2668796 |
| AA682910 | 2668801 |
| AA683050 | 2668941 |
| AA683075 | 2668966 |
| AA683077 | 2668968 |
| AA683080 | 2668971 |
| AA683085 | 2668976 |
| AA683136 | 2669027 |
| AA683246 | 2669137 |
| AA683314 | 2669205 |
| AA683338 | 2669229 |
| AA683520 | 2670118 |
| AA683546 | 2670144 |
| AA683557 | 2670155 |
| AA687179 | 2675406 |
| AA687216 | 2674421 |
| AA687336 | 2675527 |
| AA687370 | 2675561 |
| AA687495 | 2675686 |
| AA687538 | 2674444 |
| AA687571 | 2674477 |
| AA687674 | 2674580 |
| AA687675 | 2674581 |
| AA687718 | 2674624 |
| AA687733 | 2674639 |
| AA689334 | 2690433 |
| AA689392 | 2689745 |
| AA693742 | 2694680 |
| AA693922 | 2694860 |
| AA694055 | 2694993 |
| AA694120 | 2695058 |
| AA694186 | 2695124 |
| AA694271 | 2695209 |
| AA694477 | 2695415 |
| AA699390 | 2702584 |
| AA699427 | 2702621 |
| AA699442 | 2702636 |
| AA699494 | 2703650 |
| AA699560 | 2703707 |
| AA699573 | 2703720 |
| AA699601 | 2703748 |
| AA699674 | 2703821 |
| AA699707 | 2702670 |
| AA699714 | 2702677 |
| AA699719 | 2702682 |
| AA699762 | 2702725 |
| AA699770 | 2702733 |
| AA699895 | 2702858 |
| AA699926 | 2702889 |
| AA699931 | 2702894 |
| AA699999 | 2702962 |
| AA700054 | 2703017 |
| AA700218 | 2703181 |
| AA700232 | 2703195 |
| AA700415 | 2703378 |
| AA700509 | 2703472 |
| AA700599 | 2703562 |
| AA700604 | 2703567 |
| AA700680 | 2703845 |
| AA700736 | 2703901 |
| AA700739 | 2703904 |
| AA700843 | 2704008 |
| AA700876 | 2704041 |
| AA700904 | 2704069 |
| AA701034 | 2704199 |
| AA701046 | 2704211 |
| AA701315 | 2704480 |
| AA701328 | 2704493 |
| AA701351 | 2704516 |
| AA701455 | 2704620 |
| AA701467 | 2704632 |
| AA701502 | 2704667 |
| AA701519 | 2704684 |
| AA701587 | 2704752 |
| AA701607 | 2704772 |
| AA701645 | 2704810 |
| AA701652 | 2704817 |
| AA701655 | 2704820 |
| AA701667 | 2704832 |
| AA701900 | 2705013 |
| AA701931 | 2705044 |
| AA701933 | 2705046 |
| AA701948 | 2705061 |
| AA701976 | 2705089 |
| AA701978 | 2705091 |
| AA701980 | 2705093 |
| AA701981 | 2705094 |
| AA701996 | 2705109 |
| AA702077 | 2705190 |
| AA702084 | 2705197 |
| AA702131 | 2705244 |
| AA702193 | 2705306 |
| AA702230 | 2705343 |
| AA702247 | 2705360 |
| AA702327 | 2705440 |
| AA702335 | 2705448 |
| AA702398 | 2705511 |
| AA702422 | 2705535 |
| AA702480 | 2705593 |
| AA702640 | 2705753 |
| AA702679 | 2705792 |
| AA702684 | 2705797 |
| AA702738 | 2705851 |
| AA702781 | 2705894 |
| AA702785 | 2705898 |
| AA702788 | 2705901 |
| AA702802 | 2705915 |
| AA702803 | 2705916 |
| AA702888 | 2706001 |
| AA702891 | 2706004 |
| AA702942 | 2706055 |
| AA702973 | 2706086 |
| AA703000 | 2706113 |
| AA703004 | 2706117 |
| AA703046 | 2706159 |
| AA703048 | 2706161 |
| AA703079 | 2706192 |
| AA703114 | 2706227 |
| AA703117 | 2706230 |
| AA703147 | 2706260 |
| AA703159 | 2706272 |
| AA703161 | 2706274 |
| AA703219 | 2706332 |
| AA703249 | 2706362 |
| AA703387 | 2713305 |
| AA703396 | 2713314 |
| AA703652 | 2713570 |
| AA703660 | 2713578 |
| AA703816 | 2713734 |
| AA703838 | 2713756 |
| AA703925 | 2713843 |
| AA704005 | 2713923 |
| AA704255 | 2714173 |
| AA704278 | 2714196 |
| AA704323 | 2714241 |
| AA704332 | 2714250 |
| AA704370 | 2714288 |
| AA704459 | 2714377 |
| AA704537 | 2714455 |
| AA704587 | 2714505 |
| AA704650 | 2714568 |
| AA704690 | 2714608 |
| AA704718 | 2714636 |
| AA704749 | 2714667 |
| AA704777 | 2714695 |
| AA704802 | 2714720 |
| AA704816 | 2714734 |
| AA704844 | 2714762 |
| AA704858 | 2714776 |
| AA704908 | 2714826 |
| AA704943 | 2714861 |
| AA705041 | 2714959 |
| AA705047 | 2714965 |
| AA705054 | 2714972 |
| AA705058 | 2714976 |
| AA705061 | 2714979 |
| AA705072 | 2714990 |
| AA705077 | 2714995 |
| AA705103 | 2715021 |
| AA705142 | 2715060 |
| AA705184 | 2715102 |
| AA705237 | 2715155 |
| AA705258 | 2715176 |
| AA705269 | 2715187 |
| AA705319 | 2715237 |
| AA705376 | 2715294 |
| AA705433 | 2715351 |
| AA705447 | 2715365 |
| AA705516 | 2715434 |
| AA705525 | 2715443 |
| AA705546 | 2715464 |
| AA705736 | 2715654 |
| AA705743 | 2715661 |
| AA705813 | 2715731 |
| AA705819 | 2715737 |
| AA705825 | 2715743 |
| AA705840 | 2715758 |
| AA705858 | 2715776 |
| AA705957 | 2715875 |
| AA705986 | 2715904 |
| AA706018 | 2715936 |
| AA706224 | 2716142 |
| AA706241 | 2716159 |
| AA706314 | 2716232 |
| AA706388 | 2716306 |
| AA706738 | 2716656 |
| AA706764 | 2716682 |
| AA706795 | 2716713 |
| AA706797 | 2716715 |
| AA706829 | 2716747 |
| AA706839 | 2716757 |
| AA706860 | 2716778 |
| AA706870 | 2716788 |
| AA706901 | 2716819 |
| AA706935 | 2716853 |
| AA706955 | 2716873 |
| AA706964 | 2716882 |
| AA706968 | 2716886 |
| AA706969 | 2716887 |
| AA707008 | 2716926 |
| AA707080 | 2716998 |
| AA707086 | 2717004 |
| AA707118 | 2717036 |
| AA707121 | 2717039 |
| AA707125 | 2717043 |
| AA707219 | 2717137 |
| AA707336 | 2717254 |
| AA707377 | 2717295 |
| AA707400 | 2717318 |
| AA707450 | 2717368 |
| AA707490 | 2717408 |
| AA707511 | 2717429 |
| AA707607 | 2717525 |
| AA707615 | 2717533 |
| AA707659 | 2717577 |
| AA707750 | 2717668 |
| AA707753 | 2717671 |
| AA707794 | 2717712 |
| AA707840 | 2717758 |
| AA707871 | 2717789 |
| AA707872 | 2717790 |
| AA707935 | 2717853 |
| AA708003 | 2717921 |
| AA708016 | 2717934 |
| AA708143 | 2718061 |
| AA708152 | 2718070 |
| AA708189 | 2718107 |
| AA708195 | 2718113 |
| AA708201 | 2718119 |
| AA708240 | 2718158 |
| AA708342 | 2718260 |
| AA708348 | 2718266 |
| AA708431 | 2718349 |
| AA708438 | 2718356 |
| AA708440 | 2718358 |
| AA708441 | 2718359 |
| AA708446 | 2718364 |
| AA708457 | 2718375 |
| AA708501 | 2718419 |
| AA708605 | 2718523 |
| AA708676 | 2718594 |
| AA708686 | 2718604 |
| AA708690 | 2718608 |
| AA708958 | 2718876 |
| AA709044 | 2718962 |
| AA709070 | 2718988 |
| AA709208 | 2719126 |
| AA709271 | 2719189 |
| AA709333 | 2719251 |
| AA709370 | 2719288 |
| AA709388 | 2719306 |
| AA713504 | 2725778 |
| AA713517 | 2725791 |
| AA713530 | 2725804 |
| AA713580 | 2725854 |
| AA713687 | 2725961 |
| AA713760 | 2726034 |
| AA714022 | 2726296 |
| AA714481 | 2726755 |
| AA714567 | 2726841 |
| AA714902 | 2727176 |
| AA714934 | 2727208 |
| AA715000 | 2727274 |
| AA715036 | 2727310 |
| AA715365 | 2727639 |
| AA716097 | 2728371 |
| AA716414 | 2728688 |
| AA716751 | 2729025 |
| AA718910 | 2732009 |
| AA719257 | 2732356 |
| AA719530 | 2732629 |
| AA719797 | 2732896 |
| AA720578 | 2736713 |
| AA721298 | 2737433 |
| AA721375 | 2737510 |
| AA722090 | 2739797 |
| AA722204 | 2739911 |
| AA722823 | 2740530 |
| AA723045 | 2740752 |
| AA723130 | 2740837 |
| AA723521 | 2741228 |
| AA723525 | 2741232 |
| AA723584 | 2741291 |
| AA723679 | 2741386 |
| AA724392 | 2742099 |
| AA724553 | 2742260 |
| AA724619 | 2742326 |
| AA724868 | 2742575 |
| AA725298 | 2743005 |
| AA725397 | 2743104 |
| AA725400 | 2743107 |
| AA725525 | 2743232 |
| AA725564 | 2743271 |
| AA725836 | 2743543 |
| AA729119 | 2750478 |
| AA730111 | 2751393 |
| AA730534 | 2751738 |
| AA730780 | 2751984 |
| AA731507 | 2753663 |
| AA731537 | 2753693 |
| AA731556 | 2753712 |
| AA731700 | 2752589 |
| AA731872 | 2752683 |
| AA732326 | 2752933 |
| AA732352 | 2752959 |
| AA732538 | 2753145 |
| AA732832 | 2775279 |
| AA733027 | 2754386 |
| AA733038 | 2775295 |
| AA733061 | 2775300 |
| AA733090 | 2754449 |
| AA733105 | 2775307 |
| AA733177 | 2754536 |
| AA736613 | 2767847 |
| AA737421 | 2768178 |
| AA737949 | 2768706 |
| AA740151 | 2778743 |
| AA740261 | 2778853 |
| AA740574 | 2779166 |
| AA740610 | 2779202 |
| AA740911 | 2779503 |
| AA741050 | 2779642 |
| AA741056 | 2779648 |
| AA741297 | 2779889 |
| AA742189 | 2784189 |
| AA742231 | 2784231 |
| AA742522 | 2782104 |
| AA742701 | 2782207 |
| AA743453 | 2782959 |
| AA743621 | 2783127 |
| AA743680 | 2784496 |
| AA743710 | 2784526 |
| AA743975 | 2784725 |
| AA744239 | 2784989 |
| AA744367 | 2785117 |
| AA744771 | 2783535 |
| AA744849 | 2783613 |
| AA744876 | 2783640 |
| AA745059 | 2783823 |
| AA745616 | 2785602 |
| AA745678 | 2785664 |
| AA745694 | 2785680 |
| AA746353 | 2786339 |
| AA746487 | 2786473 |
| AA746824 | 2786810 |
| AA747091 | 2787049 |
| AA747515 | 2787473 |
| AA747772 | 2787730 |
| AA747819 | 2787777 |
| AA747909 | 2787867 |
| AA748031 | 2787989 |
| AA748033 | 2787991 |
| AA748038 | 2787996 |
| AA748064 | 2788022 |
| AA748444 | 2788402 |
| AA748525 | 2788483 |
| AA748847 | 2788805 |
| AA749045 | 2789003 |
| AA757282 | 2805145 |
| AA757401 | 2805264 |
| AA757417 | 2805280 |
| AA757564 | 2805427 |
| AA757588 | 2805451 |
| AA757604 | 2805467 |
| AA757659 | 2805522 |
| AA757711 | 2805574 |
| AA757717 | 2805580 |
| AA757732 | 2805595 |
| AA757754 | 2805617 |
| AA757764 | 2805627 |
| AA758023 | 2805886 |
| AA758154 | 2806017 |
| AA758348 | 2806211 |
| AA758375 | 2806238 |
| AA758470 | 2806333 |
| AA758592 | 2806455 |
| AA759202 | 2807065 |
| AA760738 | 2809668 |
| AA760894 | 2809824 |
| AA761069 | 2809999 |
| AA761562 | 2810492 |
| AA761640 | 2810570 |
| AA761739 | 2810669 |
| AA761831 | 2810761 |
| AA764981 | 2816219 |
| AA764991 | 2816229 |
| AA765140 | 2816378 |
| AA765712 | 2816950 |
| AA765884 | 2817122 |
| AA765909 | 2817147 |
| AA765947 | 2817185 |
| AA765974 | 2817212 |
| AA766050 | 2817288 |
| AA766644 | 2817882 |
| AA766817 | 2818055 |
| AA767154 | 2819735 |
| AA767323 | 2818338 |
| AA767439 | 2818454 |
| AA767457 | 2818472 |
| AA767764 | 2818779 |
| AA767779 | 2818794 |
| AA767784 | 2818799 |
| AA768109 | 2819124 |
| AA768348 | 2819363 |
| AA768762 | 2819477 |
| AA768951 | 2820189 |
| AA769055 | 2820293 |
| AA769102 | 2820340 |
| AA769127 | 2820365 |
| AA769142 | 2820380 |
| AA769434 | 2820672 |
| AA769696 | 2820934 |
| AA769722 | 2820960 |
| AA769865 | 2821103 |
| AA769937 | 2821175 |
| AA770006 | 2821244 |
| AA770272 | 2821510 |
| AA770505 | 2821743 |
| AA770690 | 2821928 |
| AA772075 | 2823858 |
| AA772465 | 2824248 |
| AA772497 | 2824280 |
| AA772603 | 2824386 |
| AA772683 | 2824466 |
| AA772736 | 2824519 |
| AA772803 | 2825645 |
| AA773009 | 2824580 |
| AA773072 | 2824643 |
| AA773139 | 2824710 |
| AA773362 | 2824933 |
| AA773374 | 2824945 |
| AA773478 | 2825049 |
| AA773641 | 2825212 |
| AA773787 | 2825358 |
| AA773998 | 2825887 |
| AA774230 | 2825528 |
| AA774247 | 2825545 |
| AA774265 | 2825563 |
| AA774501 | 2833835 |
| AA774523 | 2833857 |
| AA774645 | 2833979 |
| AA774665 | 2833999 |
| AA774678 | 2834012 |
| AA774750 | 2834084 |
| AA774869 | 2834203 |
| AA775223 | 2834557 |
| AA775257 | 2834591 |
| AA775259 | 2834593 |
| AA775325 | 2834659 |
| AA775355 | 2834689 |
| AA775405 | 2834739 |
| AA775422 | 2834756 |
| AA775431 | 2834765 |
| AA775447 | 2834781 |
| AA775557 | 2834891 |
| AA775616 | 2834950 |
| AA775625 | 2834959 |
| AA775774 | 2835108 |
| AA775791 | 2835125 |
| AA775810 | 2835144 |
| AA775824 | 2835158 |
| AA775857 | 2835191 |
| AA775863 | 2835197 |
| AA775865 | 2835199 |
| AA775888 | 2835222 |
| AA775914 | 2835248 |
| AA775918 | 2835252 |
| AA775957 | 2835291 |
| AA776041 | 2835375 |
| AA776434 | 2835768 |
| AA776585 | 2835919 |
| AA776692 | 2836026 |
| AA776702 | 2836036 |
| AA776709 | 2836043 |
| AA776740 | 2836074 |
| AA776755 | 2836089 |
| AA776810 | 2836141 |
| AA777024 | 2836355 |
| AA777092 | 2836423 |
| AA777098 | 2836429 |
| AA777187 | 2836518 |
| AA777265 | 2836596 |
| AA777394 | 2836725 |
| AA777417 | 2836748 |
| AA777488 | 2836967 |
| AA777489 | 2836968 |
| AA777529 | 2837008 |
| AA777536 | 2837015 |
| AA777555 | 2837034 |
| AA777590 | 2837069 |
| AA777670 | 2837149 |
| AA777696 | 2837175 |
| AA777700 | 2837179 |
| AA777705 | 2837184 |
| AA777779 | 2837258 |
| AA777781 | 2837260 |
| AA777883 | 2836876 |
| AA777886 | 2836879 |
| AA777902 | 2837303 |
| AA778038 | 2837439 |
| AA778089 | 2837490 |
| AA778098 | 2837499 |
| AA778109 | 2837510 |
| AA778206 | 2836921 |
| AA778533 | 2837864 |
| AA778562 | 2837893 |
| AA778571 | 2837902 |
| AA778596 | 2837927 |
| AA778603 | 2837934 |
| AA778636 | 2837967 |
| AA778646 | 2837977 |
| AA778653 | 2837984 |
| AA778663 | 2837994 |
| AA778675 | 2838006 |
| AA778717 | 2838048 |
| AA778839 | 2838170 |
| AA778869 | 2838200 |
| AA779048 | 2838379 |
| AA779165 | 2838496 |
| AA779176 | 2838507 |
| AA779212 | 2838543 |
| AA779221 | 2838552 |
| AA779225 | 2838556 |
| AA779278 | 2838609 |
| AA779380 | 2838711 |
| AA779383 | 2838714 |
| AA779391 | 2838722 |
| AA779427 | 2838758 |
| AA779457 | 2838788 |
| AA779602 | 2838933 |
| AA779670 | 2839001 |
| AA779728 | 2839059 |
| AA779919 | 2839250 |
| AA779949 | 2839280 |
| AA779968 | 2839299 |
| AA779972 | 2839303 |
| AA780027 | 2839358 |
| AA780190 | 2839521 |
| AA780301 | 2839632 |
| AA780365 | 2839696 |
| AA780591 | 2839922 |
| AA780695 | 2840026 |
| AA780841 | 2840172 |
| AA781086 | 2840417 |
| AA782138 | 2841469 |
| AA782292 | 2841623 |
| AA782380 | 2841711 |
| AA782483 | 2841814 |
| AA782592 | 2841923 |
| AA782644 | 2841975 |
| AA782769 | 2842100 |
| AA788738 | 2848858 |
| AA788779 | 2848899 |
| AA788780 | 2848900 |
| AA788875 | 2848995 |
| AA788970 | 2849090 |
| AA789328 | 2849448 |
| AA804375 | 2875888 |
| AA804544 | 2873675 |
| AA804659 | 2876060 |
| AA804740 | 2876095 |
| AA804879 | 2876206 |
| AA805228 | 2876380 |
| AA805361 | 2874111 |
| AA805604 | 2874354 |
| AA805865 | 2874615 |
| AA806040 | 2874790 |
| AA806057 | 2874807 |
| AA806060 | 2874810 |
| AA806448 | 2875198 |
| AA806555 | 2875305 |
| AA806717 | 2875467 |
| AA806808 | 2875558 |
| AA807276 | 2876852 |
| AA807370 | 2876946 |
| AA808003 | 2877409 |
| AA808146 | 2877552 |
| AA808211 | 2877617 |
| AA808349 | 2877755 |
| AA808466 | 2877872 |
| AA808492 | 2877898 |
| AA808582 | 2877988 |
| AA809784 | 2879190 |
| AA810159 | 2879565 |
| AA810225 | 2879584 |
| AA810340 | 2879699 |
| AA810418 | 2875690 |
| AA810905 | 2880516 |
| AA811333 | 2880944 |
| AA811365 | 2880976 |
| AA812081 | 2881692 |
| AA812193 | 2881804 |
| AA812209 | 2881820 |
| AA812581 | 2882645 |
| AA812676 | 2882740 |
| AA812993 | 2883057 |
| AA812996 | 2883060 |
| AA813108 | 2883093 |
| AA813266 | 2883251 |
| AA813287 | 2883272 |
| AA813373 | 2883358 |
| AA813708 | 2882393 |
| AA813941 | 2883537 |
| AA814091 | 2883687 |
| AA814412 | 2884008 |
| AA814414 | 2884010 |
| AA814496 | 2884092 |
| AA814500 | 2884096 |
| AA814634 | 2884230 |
| AA814822 | 2884418 |
| AA814859 | 2884455 |
| AA815173 | 2884769 |
| AA815305 | 2884901 |
| AA824395 | 2896279 |
| AA824401 | 2896285 |
| AA824416 | 2896438 |
| AA825194 | 2898491 |
| AA825265 | 2898566 |
| AA825512 | 2898824 |
| AA825612 | 2898924 |
| AA825768 | 2899080 |
| AA825940 | 2899252 |
| AA826237 | 2899549 |
| AA826244 | 2899556 |
| AA826324 | 2899636 |
| AA826464 | 2898281 |
| AA826491 | 2898311 |
| AA826649 | 2900646 |
| AA826906 | 2900903 |
| AA827079 | 2901076 |
| AA827196 | 2901193 |
| AA827331 | 2899772 |
| AA827369 | 2899810 |
| AA827405 | 2899846 |
| AA827738 | 2901297 |
| AA827816 | 2900179 |
| AA827875 | 2900238 |
| AA827878 | 2900241 |
| AA827968 | 2900331 |
| AA828243 | 2901342 |
| AA828708 | 2901807 |
| AA829236 | 2902335 |
| AA829350 | 2902449 |
| AA829473 | 2902572 |
| AA829547 | 2902646 |
| AA829850 | 2902949 |
| AA829973 | 2903072 |
| AA830635 | 2903734 |
| AA830807 | 2903906 |
| AA830856 | 2903955 |
| AA830959 | 2904058 |
| AA831078 | 2904177 |
| AA831148 | 2904247 |
| AA831278 | 2904377 |
| AA831795 | 2904894 |
| AA831876 | 2904975 |
| AA832480 | 2905579 |
| AA833543 | 2907271 |
| AA833759 | 2908527 |
| AA834102 | 2907701 |
| AA834540 | 2908139 |
| AA834946 | 2908674 |
| AA835377 | 2909105 |
| AA835490 | 2909218 |
| AA835638 | 2909957 |
| AA835708 | 2910027 |
| AA836233 | 2910552 |
| AA836306 | 2910625 |
| AA836488 | 2910807 |
| AA836682 | 2909940 |
| AA836834 | 2912033 |
| AA836962 | 2912161 |
| AA837048 | 2912247 |
| AA837401 | 2912600 |
| AA837457 | 2912656 |
| AA838003 | 2913660 |
| AA838377 | 2913176 |
| AA838410 | 2913209 |
| AA838431 | 2913230 |
| AA838482 | 2913281 |
| AA838681 | 2914793 |
| AA838691 | 2914803 |
| AA838697 | 2914809 |
| AA838748 | 2914860 |
| AA843133 | 2929651 |
| AA843176 | 2929694 |
| AA843216 | 2929734 |
| AA843242 | 2929760 |
| AA843326 | 2929844 |
| AA843414 | 2929932 |
| AA843531 | 2930049 |
| AA843592 | 2930110 |
| AA843663 | 2930181 |
| AA843683 | 2933039 |
| AA843706 | 2933062 |
| AA843845 | 2930296 |
| AA844070 | 2930521 |
| AA844081 | 2930532 |
| AA844124 | 2930575 |
| AA844141 | 2930592 |
| AA844230 | 2930681 |
| AA844447 | 2930898 |
| AA844734 | 2931185 |
| AA844979 | 2931430 |
| AA844998 | 2931449 |
| AA845156 | 2931607 |
| AA845168 | 2931619 |
| AA845340 | 2933099 |
| AA845374 | 2933133 |
| AA845390 | 2933149 |
| AA845409 | 2933168 |
| AA845442 | 2933201 |
| AA845575 | 2933334 |
| AA845584 | 2933343 |
| AA846152 | 2932292 |
| AA846308 | 2932448 |
| AA846335 | 2932475 |
| AA846576 | 2932716 |
| AA846626 | 2932766 |
| AA847210 | 2933728 |
| AA847348 | 2933866 |
| AA847380 | 2933898 |
| AA847452 | 2933970 |
| AA847597 | 2934115 |
| AA847656 | 2934174 |
| AA848129 | 2934647 |
| AA852908 | 2939647 |
| AA852998 | 2939737 |
| AA853183 | 2939922 |
| AA853882 | 2940621 |
| AA854071 | 2941609 |
| AA854293 | 2941831 |
| AA854305 | 2941843 |
| AA854319 | 2941857 |
| AA855093 | 2942631 |
| AA855158 | 2942696 |
| AA856556 | 2944858 |
| AA856719 | 2945021 |
| AA856914 | 2945216 |
| AA857035 | 2945337 |
| AA857069 | 2945371 |
| AA857163 | 2945465 |
| AA857212 | 2945514 |
| AA857413 | 2945715 |
| AA857476 | 2945778 |
| AA857478 | 2945780 |
| AA857496 | 2945798 |
| AA857509 | 2945811 |
| AA857632 | 2945934 |
| AA857748 | 2946050 |
| AA857804 | 2946106 |
| AA857809 | 2946111 |
| AA857872 | 2946174 |
| AA857936 | 2946238 |
| AA858389 | 2946691 |
| AA858390 | 2946692 |
| AA858396 | 2946698 |
| AA860246 | 2954241 |
| AA860287 | 2954282 |
| AA860541 | 2952681 |
| AA860839 | 2952979 |
| AA860988 | 2953126 |
| AA861428 | 2953568 |
| AA861631 | 2953771 |
| AA861930 | 2952554 |
| AA861986 | 2954465 |
| AA862250 | 2954729 |
| AA862309 | 2954788 |
| AA862465 | 2954944 |
| AA862814 | 2955293 |
| AA862821 | 2955300 |
| AA862855 | 2955334 |
| AA862992 | 2955471 |
| AA863125 | 2955604 |
| AA863169 | 2955648 |
| AA863204 | 2955683 |
| AA863292 | 2955771 |
| AA863314 | 2955793 |
| AA863393 | 2955872 |
| AA863449 | 2955928 |
| AA864406 | 2958719 |
| AA864681 | 2958994 |
| AA864704 | 2959017 |
| AA864840 | 2959153 |
| AA864875 | 2959188 |
| AA864975 | 2959288 |
| AA865150 | 2957426 |
| AA865185 | 2957461 |
| AA865464 | 2957740 |
| AA865873 | 2958149 |
| AA865913 | 2958189 |
| AA865924 | 2958200 |
| AA866029 | 2958305 |
| AA866035 | 2958311 |
| AA868038 | 2963483 |
| AA868337 | 2963782 |
| AA868512 | 2963957 |
| AA868515 | 2963960 |
| AA868720 | 2964165 |
| AA868802 | 2964247 |
| AA868929 | 2964374 |
| AA872006 | 2968044 |
| AA872034 | 2968072 |
| AA872057 | 2968235 |
| AA872122 | 2968300 |
| AA872143 | 2968321 |
| AA872153 | 2968331 |
| AA872257 | 2968435 |
| AA872323 | 2968501 |
| AA872383 | 2968561 |
| AA872397 | 2968575 |
| AA872402 | 2968580 |
| AA872436 | 2968614 |
| AA872454 | 2968632 |
| AA872602 | 2968780 |
| AA872606 | 2968784 |
| AA872692 | 2968132 |
| AA872704 | 2968144 |
| AA872710 | 2968832 |
| AA872729 | 2968851 |
| AA872858 | 2968980 |
| AA872969 | 2969091 |
| AA872979 | 2969101 |
| AA873060 | 2969182 |
| AA873089 | 2969211 |
| AA873110 | 2969232 |
| AA873159 | 2969281 |
| AA873182 | 2969304 |
| AA873291 | 2969413 |
| AA873351 | 2969473 |
| AA873355 | 2969477 |
| AA873459 | 2969581 |
| AA873534 | 2969656 |
| AA873577 | 2969699 |
| AA873578 | 2969700 |
| AA873604 | 2969726 |
| AA873635 | 2969757 |
| AA873762 | 2968148 |
| AA873828 | 2968214 |
| AA875888 | 2985247 |
| AA875913 | 2985272 |
| AA875936 | 2985295 |
| AA875953 | 2985312 |
| AA876021 | 2984862 |
| AA876354 | 2985431 |
| AA876375 | 2985452 |
| AA876384 | 2985461 |
| AA876439 | 2985516 |
| AA876668 | 2985745 |
| AA876730 | 2985807 |
| AA876897 | 2985974 |
| AA877347 | 2986424 |
| AA877460 | 2985167 |
| AA877789 | 2986754 |
| AA877796 | 2986761 |
| AA877815 | 2986780 |
| AA877864 | 2986829 |
| AA877997 | 2986962 |
| AA878120 | 2987085 |
| AA878195 | 2987160 |
| AA878210 | 2987175 |
| AA878211 | 2987176 |
| AA878212 | 2987177 |
| AA878395 | 2987360 |
| AA878561 | 2987526 |
| AA878731 | 2987696 |
| AA878739 | 2987704 |
| AA878781 | 2987746 |
| AA878880 | 2987845 |
| AA879423 | 2988534 |
| AA879435 | 2988546 |
| AA879437 | 2988548 |
| AA879474 | 2988585 |
| AA883100 | 2992699 |
| AA883114 | 2992713 |
| AA883127 | 2992726 |
| AA883132 | 2994608 |
| AA883160 | 2994636 |
| AA883170 | 2994646 |
| AA883181 | 2994657 |
| AA883187 | 2994663 |
| AA883299 | 2992829 |
| AA883391 | 2992921 |
| AA883431 | 2992961 |
| AA883504 | 2993034 |
| AA883508 | 2993038 |
| AA883580 | 2993110 |
| AA883612 | 2993142 |
| AA883660 | 2993190 |
| AA883679 | 2993209 |
| AA883680 | 2993210 |
| AA883711 | 2993241 |
| AA883735 | 2993265 |
| AA883755 | 2993285 |
| AA883800 | 2993330 |
| AA883901 | 2993431 |
| AA883914 | 2993444 |
| AA884098 | 2993628 |
| AA884119 | 2993649 |
| AA884249 | 2993779 |
| AA884317 | 2993847 |
| AA884321 | 2993851 |
| AA884382 | 2993912 |
| AA884397 | 2993927 |
| AA884428 | 2993958 |
| AA884622 | 2994152 |
| AA884713 | 2994694 |
| AA884897 | 2994878 |
| AA884922 | 2994903 |
| AA884926 | 2994907 |
| AA884972 | 2994953 |
| AA884992 | 2994973 |
| AA885052 | 2995033 |
| AA885096 | 2995077 |
| AA885140 | 2995121 |
| AA885141 | 2995122 |
| AA885293 | 2994370 |
| AA885311 | 2994388 |
| AA885470 | 2994547 |
| AA885471 | 2994548 |
| AA885478 | 2994555 |
| AA885642 | 3000750 |
| AA885770 | 3000878 |
| AA885780 | 3000888 |
| AA885819 | 3000927 |
| AA885835 | 3000943 |
| AA885851 | 3000959 |
| AA885902 | 3001010 |
| AA886333 | 3001441 |
| AA886406 | 3001514 |
| AA886497 | 3001605 |
| AA886739 | 3001847 |
| AA886748 | 3001856 |
| AA886792 | 3001900 |
| AA886818 | 3001926 |
| AA886870 | 3001978 |
| AA886957 | 3002065 |
| AA886998 | 3002106 |
| AA887054 | 3002162 |
| AA887201 | 3002309 |
| AA887224 | 3002332 |
| AA887235 | 3002343 |
| AA888014 | 3003689 |
| AA888121 | 3003796 |
| AA888589 | 3004264 |
| AA888665 | 3004340 |
| AA889397 | 3016276 |
| AA889785 | 3016664 |
| AA889969 | 3016848 |
| AA890010 | 3016889 |
| AA890032 | 3016911 |
| AA890098 | 3016977 |
| AA890147 | 3017026 |
| AA890206 | 3017085 |
| AA890326 | 3017205 |
| AA890327 | 3017206 |
| AA890421 | 3017300 |
| AA890660 | 3017539 |
| AA894408 | 3030809 |
| AA894557 | 3030958 |
| AA894577 | 3030978 |
| AA894687 | 3031088 |
| AA894781 | 3031182 |
| AA894929 | 3031330 |
| AA896959 | 3033352 |
| AA902127 | 3037317 |
| AA902249 | 3037439 |
| AA902256 | 3037446 |
| AA902388 | 3037511 |
| AA902625 | 3037748 |
| AA902721 | 3037844 |
| AA902831 | 3037954 |
| AA902953 | 3038076 |
| AA902964 | 3038087 |
| AA903139 | 3038262 |
| AA903809 | 3038932 |
| AA904265 | 3039388 |
| AA904647 | 3039770 |
| AA904776 | 3039899 |
| AA904969 | 3040092 |
| AA905065 | 3040188 |
| AA905139 | 3040262 |
| AA905838 | 3040961 |
| AA905896 | 3041019 |
| AA906257 | 3041380 |
| AA906582 | 3042168 |
| AA906652 | 3042238 |
| AA906868 | 3042112 |
| AA906997 | 3042457 |
| AA907003 | 3042463 |
| AA907068 | 3042528 |
| AA907575 | 3043035 |
| AA907660 | 3043120 |
| AA907714 | 3043174 |
| AA908797 | 3048202 |
| AA908930 | 3048335 |
| AA908961 | 3048366 |
| AA909045 | 3048450 |
| AA909768 | 3050567 |
| AA909977 | 3049267 |
| AA910369 | 3049659 |
| AA911154 | 3050444 |
| AA911236 | 3050526 |
| AA911245 | 3050535 |
| AA911317 | 3050681 |
| AA911903 | 3051295 |
| AA911971 | 3051363 |
| AA912071 | 3051463 |
| AA912233 | 3051625 |
| AA912713 | 3052105 |
| AA912796 | 3052188 |
| AA913138 | 3052530 |
| AA916145 | 3055537 |
| AA916323 | 3055715 |
| AA916325 | 3055717 |
| AA916635 | 3056027 |
| AA916753 | 3056145 |
| AA917666 | 3057556 |
| AA917937 | 3057827 |
| AA918023 | 3057913 |
| AA918370 | 3058260 |
| AA918993 | 3058883 |
| AA921906 | 3069215 |
| AA922237 | 3069546 |
| AA922309 | 3069618 |
| AA922326 | 3069635 |
| AA923172 | 3070481 |
| AA923278 | 3070587 |
| AA923547 | 3070856 |
| AA926656 | 3075553 |
| AA926926 | 3075823 |
| AA926992 | 3075889 |
| AA927118 | 3076015 |
| AA927433 | 3076330 |
| AA927544 | 3076441 |
| AA927818 | 3077108 |
| AA928420 | 3077576 |
| AA928427 | 3077583 |
| AA931164 | 3085550 |
| AA931228 | 3085614 |
| AA931237 | 3085623 |
| AA931583 | 3085969 |
| AA931666 | 3086052 |
| AA931725 | 3086111 |
| AA931943 | 3086329 |
| AA932059 | 3086372 |
| AA932114 | 3086427 |
| AA932252 | 3087164 |
| AA932331 | 3086569 |
| AA932402 | 3087183 |
| AA932569 | 3087350 |
| AA933573 | 3089841 |
| AA933627 | 3089895 |
| AA933888 | 3090156 |
| AA934368 | 3091525 |
| AA934530 | 3091687 |
| AA934755 | 3091967 |
| AA934762 | 3091974 |
| AA934851 | 3092063 |
| AA934879 | 3092091 |
| AA935181 | 3092338 |
| AA935485 | 3092642 |
| AA935526 | 3092683 |
| AA935560 | 3092717 |
| AA935798 | 3092955 |
| AA935811 | 3092968 |
| AA935845 | 3093002 |
| AA936089 | 3093246 |
| AA936187 | 3094105 |
| AA936262 | 3094180 |
| AA936532 | 3094450 |
| AA936783 | 3094817 |
| AA936792 | 3094826 |
| AA937212 | 3095323 |
| AA937513 | 3095624 |
| AA937551 | 3095662 |
| AA937773 | 3095884 |
| AA938156 | 3096267 |
| AA938181 | 3096292 |
| AA938717 | 3096745 |
| AA939023 | 3098936 |
| AA939199 | 3099112 |
| AA939266 | 3099179 |
| AA939275 | 3099188 |
| AA946679 | 3110074 |
| AA946873 | 3110268 |
| AA946883 | 3110278 |
| AA946995 | 3110390 |
| AA947278 | 3108531 |
| AA947441 | 3108694 |
| AA947511 | 3108764 |
| AA947922 | 3109175 |
| AA948037 | 3109290 |
| AA948223 | 3109476 |
| AA948276 | 3109529 |
| AA948320 | 3109573 |
| AA953229 | 3117376 |
| AA953328 | 3117475 |
| AA953357 | 3117504 |
| AA953391 | 3117538 |
| AA953892 | 3116810 |
| AA953973 | 3116891 |
| AA954079 | 3116997 |
| AA954118 | 3117813 |
| AA954674 | 3118369 |
| AA954939 | 3118634 |
| AA955007 | 3118702 |
| AA960820 | 3127374 |
| AA960924 | 3127478 |
| AA961085 | 3127639 |
| AA961361 | 3133534 |
| AA962119 | 3134283 |
| AA962236 | 3134400 |
| AA962300 | 3134464 |
| AA962541 | 3134705 |
| AA962550 | 3134714 |
| AA962587 | 3134751 |
| AA968556 | 3143736 |
| AA968621 | 3143801 |
| AA968756 | 3143936 |
| AA968896 | 3144076 |
| AA969184 | 3144364 |
| AA969214 | 3144394 |
| AA969459 | 3144639 |
| AA969512 | 3144692 |
| AA969613 | 3144793 |
| AA970266 | 3145779 |
| AA970314 | 3145827 |
| AA970570 | 3145077 |
| AA970720 | 3146010 |
| AA971532 | 3146822 |
| AA971641 | 3146931 |
| AA971698 | 3146988 |
| AA971700 | 3146990 |
| AA972352 | 3147642 |
| AA972530 | 3145294 |
| AA972598 | 3147778 |
| AA972602 | 3147782 |
| AA972625 | 3147805 |
| AA972792 | 3147972 |
| AA972883 | 3148063 |
| AA973273 | 3148453 |
| AA973560 | 3148740 |
| AA973620 | 3148800 |
| AA973927 | 3149107 |
| AA974137 | 3149317 |
| AA974348 | 3149528 |
| AA974402 | 3149582 |
| AA974495 | 3149675 |
| AA974540 | 3149720 |
| AA974621 | 3149801 |
| AA974826 | 3150618 |
| AA974908 | 3150700 |
| AA974971 | 3150763 |
| AA974999 | 3150791 |
| AA975010 | 3150802 |
| AA975209 | 3151001 |
| AA975275 | 3151067 |
| AA975388 | 3151180 |
| AA975556 | 3151348 |
| AA976063 | 3151855 |
| AA976544 | 3153990 |
| AA976561 | 3154007 |
| AA976714 | 3154160 |
| AA977172 | 3154618 |
| AA977197 | 3154643 |
| AA977226 | 3154672 |
| AA977296 | 3154742 |
| AA977406 | 3154852 |
| AA977833 | 3155279 |
| AA977871 | 3155317 |
| AA978126 | 3155572 |
| AA978214 | 3155660 |
| AA978353 | 3153962 |
| AA983362 | 3161887 |
| AA983467 | 3161992 |
| AA983560 | 3162085 |
| AA983832 | 3162357 |
| AA984043 | 3162568 |
| AA984134 | 3162659 |
| AA984173 | 3162698 |
| AA984384 | 3162909 |
| AA984407 | 3162932 |
| AA984460 | 3162985 |
| AA984586 | 3163111 |
| AA984690 | 3163215 |
| AA984744 | 3163269 |
| AA985190 | 3163715 |
| AA985327 | 3163852 |
| AA987290 | 3172654 |
| AA987442 | 3172806 |
| AA987644 | 3173008 |
| AA987997 | 3173361 |
| AA988011 | 3173375 |
| AA988102 | 3173466 |
| AA988125 | 3173489 |
| AA988345 | 3174037 |
| AA988468 | 3174160 |
| AA988520 | 3174212 |
| AA988630 | 3173621 |
| AA988701 | 3173692 |
| AA988746 | 3174317 |
| AA988798 | 3174369 |
| AA988920 | 3174491 |
| AA989049 | 3174620 |
| AA989139 | 3173761 |
| AA989210 | 3173832 |
| AA989473 | 3174837 |
| AA991196 | 3177685 |
| AA991398 | 3177887 |
| AA991856 | 3178738 |
| AA992217 | 3178331 |
| AA992583 | 3179339 |
| AA992596 | 3179352 |
| AA992606 | 3179362 |
| AA992672 | 3178406 |
| AA992882 | 3179427 |
| AA993414 | 3179959 |
| AA993438 | 3179983 |
| AA993550 | 3180095 |
| AA994034 | 3180579 |
| AA994534 | 3181079 |
| AA994694 | 3181183 |
| AA994757 | 3181246 |
| AA994801 | 3181290 |
| AA994811 | 3181300 |
| AA994857 | 3181346 |
| AA994966 | 3181455 |
| AA994993 | 3181482 |
| AA995066 | 3181555 |
| AA995483 | 3181972 |
| AA995508 | 3181997 |
| AA995613 | 3182102 |
| AA995747 | 3182236 |
| AA995981 | 3182470 |
| AA996010 | 3182499 |
| AA996028 | 3182517 |
| AA996104 | 3182593 |
| AA996131 | 3182620 |
| AA996230 | 3182719 |
| AA999953 | 3190508 |
| AB000095 | 2924600 |
| AB000220 | 3426162 |
| AB000468 | 1843400 |
| AB000509 | 2982670 |
| AB000584 | 1813326 |
| AB000887 | 2189952 |
| AB000888 | 2467297 |
| AB001106 | 1850083 |
| AB001636 | 2696612 |
| AB001872 | 2879897 |
| AB002303 | 2224550 |
| AB002306 | 2224556 |
| AB002311 | 2224566 |
| AB002312 | 2224568 |
| AB002319 | 2224582 |
| AB002329 | 2224602 |
| AB002330 | 2224604 |
| AB002331 | 2224606 |
| AB002346 | 6634018 |
| AB002347 | 2224638 |
| AB002350 | 2224644 |
| AB002353 | 2224650 |
| AB002357 | 2224658 |
| AB002365 | 2224674 |
| AB002366 | 2224676 |
| AB002369 | 2224682 |
| AB002370 | 2280483 |
| AB002375 | 2280486 |
| AB002380 | 2224704 |
| AB002382 | 2224708 |
| AB002383 | 2224710 |
| AB002386 | 2224716 |
| AB002387 | 2224718 |
| AB002391 | 6683696 |
| AB002451 | 2943817 |
| AB002806 | 2780782 |
| AB003103 | 1945610 |
| AB004047 | 2116654 |
| AB004066 | 2308996 |
| AB004304 | 4176417 |
| AB004788 | 3184185 |
| AB004851 | 3702685 |
| AB004857 | 2911111 |
| AB004903 | 2443360 |
| AB005289 | 3228278 |
| AB005535 | 2326258 |
| AB005621 | 2832907 |
| AB006077 | 2564010 |
| AB006534 | 2924619 |
| AB006621 | 2564313 |
| AB006625 | 2564321 |
| AB006965 | 2385511 |
| AB006968 | 2463524 |
| AB007191 | 8698842 |
| AB007510 | 2463576 |
| AB007618 | 2465177 |
| AB007867 | 2662094 |
| AB007870 | 2662100 |
| AB007873 | 2887446 |
| AB007882 | 2887418 |
| AB007883 | 2887420 |
| AB007884 | 2887422 |
| AB007885 | 2887424 |
| AB007891 | 2887440 |
| AB007892 | 2887434 |
| AB007893 | 2887436 |
| AB007896 | 2662152 |
| AB007898 | 2662156 |
| AB007899 | 2662158 |
| AB007916 | 6683704 |
| AB007929 | 3413881 |
| AB007930 | 3413883 |
| AB007931 | 3413885 |
| AB007932 | 3413887 |
| AB007934 | 3413891 |
| AB007935 | 3413893 |
| AB007941 | 3413905 |
| AB007947 | 3413917 |
| AB007949 | 3413921 |
| AB007956 | 3413930 |
| AB007957 | 3413931 |
| PB007960 | 3413934 |
| PB007963 | 3413937 |
| PB007969 | 3413944 |
| PB007972 | 3413947 |
| AB008109 | 2554613 |
| AB008226 | 3370992 |
| AB008430 | 2766164 |
| AB008681 | 2760152 |
| AB009284 | 2723392 |
| AB009285 | 3341898 |
| AB010427 | 3337112 |
| AB010882 | 2967451 |
| AB011004 | 3273315 |
| AB011079 | 2897815 |
| AB011087 | 3043553 |
| AB011103 | 3043585 |
| AB011108 | 3043595 |
| AB011119 | 3043617 |
| AB011120 | 3043619 |
| AB011132 | 6635202 |
| AB011140 | 3043659 |
| AB011145 | 3043669 |
| AB011147 | 3043673 |
| AB011148 | 3043675 |
| AB011155 | 3043689 |
| AB011158 | 3043695 |
| AB011159 | 3043697 |
| AB011164 | 3043707 |
| AB011165 | 3043709 |
| AB011169 | 3043717 |
| AB011170 | 3043719 |
| AB011175 | 3043729 |
| AB011472 | 4519430 |
| AB012130 | 3097315 |
| AB012910 | 3721650 |
| AB013897 | 6177784 |
| AB013918 | 3410908 |
| AB014458 | 3928761 |
| AB014486 | 4062959 |
| AB014511 | 3327035 |
| AB014514 | 3327041 |
| AB014519 | 3327051 |
| AB014521 | 3327055 |
| AB014525 | 3327063 |
| AB014531 | 3327075 |
| AB014533 | 3327079 |
| AB014536 | 3327085 |
| AB014542 | 3327097 |
| AB014543 | 3327099 |
| AB014547 | 3327107 |
| AB014548 | 3327109 |
| AB014552 | 3327117 |
| AB014560 | 3327133 |
| AB014563 | 3327139 |
| AB014569 | 3327151 |
| AB014577 | 3327167 |
| AB014579 | 3327171 |
| AB014589 | 3327191 |
| AB014597 | 3327207 |
| AB014593 | 3327209 |
| AB014601 | 3327215 |
| AB014610 | 3327233 |
| AB014888 | 3402484 |
| AB015317 | 3641673 |
| AB015333 | 3970855 |
| AB015639 | 5821139 |
| AB015644 | 3986199 |
| AB015907 | 4218063 |
| AB016068 | 3721863 |
| AB016087 | 5821142 |
| AB016533 | 4586430 |
| AB017004 | 4239949 |
| AB017007 | 4239955 |
| AB017430 | 4519442 |
| AB017971 | 4520349 |
| AB018080 | 3978556 |
| AB018257 | 3882148 |
| AB018266 | 3882166 |
| AB018268 | 3882170 |
| AB018270 | 3882174 |
| AB018274 | 3882182 |
| AB018276 | 3882186 |
| AB018280 | 3882194 |
| AB018281 | 3882196 |
| AB018284 | 3882202 |
| AB018296 | 3882226 |
| AB018301 | 3882236 |
| AB018304 | 3882242 |
| AB018305 | 3882244 |
| AB018313 | 3882260 |
| AB018315 | 3882264 |
| AB018319 | 3882272 |
| AB018320 | 3882274 |
| AB018324 | 3882282 |
| AB018327 | 3882288 |
| AB018330 | 3882294 |
| AB018331 | 3882296 |
| AB018334 | 3882302 |
| AB018337 | 3882308 |
| AB018338 | 3882310 |
| AB018340 | 3882314 |
| AB018342 | 3882318 |
| AB018344 | 3882322 |
| AB018346 | 3882326 |
| AB018351 | 3882336 |
| AB018352 | 3882338 |
| AB018353 | 3882340 |
| AB018358 | 6009489 |
| AB019002 | 4587082 |
| AB019392 | 4587122 |
| AB019435 | 4760646 |
| AB019524 | 4519939 |
| AB019563 | 3885366 |
| AB019564 | 3885367 |
| AB019566 | 3885369 |
| AB019568 | 3885371 |
| AB019691 | 5051742 |
| AB019987 | 4092845 |
| AB020631 | 4240136 |
| AB020633 | 4240140 |
| AB020636 | 4240146 |
| AB020637 | 4240148 |
| AB020639 | 4240152 |
| AB020643 | 4240160 |
| AB020646 | 4240166 |
| AB020650 | 4240174 |
| AB020652 | 4240178 |
| AB020657 | 4240188 |
| AB020658 | 4240190 |
| AB020661 | 4240196 |
| AB020665 | 4240204 |
| AB020669 | 4240212 |
| AB020673 | 4240220 |
| AB020679 | 4240232 |
| AB020680 | 4240234 |
| AB020682 | 4240238 |
| AB020686 | 4240246 |
| AB020689 | 4240252 |
| AB020692 | 4240258 |
| AB020693 | 4240260 |
| AB020694 | 4240262 |
| AB020698 | 4240270 |
| AB020705 | 4240284 |
| AB020707 | 4240288 |
| AB020723 | 4240320 |
| AB020724 | 4240322 |
| AB020866 | 4003386 |
| AB020880 | 4998281 |
| AB020981 | 4996287 |
| AB021179 | 4062855 |
| AB021288 | 4038732 |
| AB021663 | 4996450 |
| AB021981 | 4903003 |
| AB022663 | 5019617 |
| AB023145 | 4589487 |
| AB023150 | 4589509 |
| AB023153 | 4589515 |
| AB023157 | 4589523 |
| AB023163 | 4589535 |
| AB023173 | 4589555 |
| AB023177 | 4589563 |
| AB023179 | 4589567 |
| AB023182 | 4589573 |
| AB023196 | 4589601 |
| AB023198 | 4589605 |
| AB023201 | 4589611 |
| AB023205 | 4589619 |
| AB023206 | 4589621 |
| AB023210 | 4589629 |
| AB023215 | 4589639 |
| AB023224 | 4589657 |
| AB023227 | 4589669 |
| AB023230 | 4589675 |
| AB024334 | 6016837 |
| AB024435 | 4520137 |
| AB024518 | 4520327 |
| AB024704 | 4589928 |
| AB026190 | 4650843 |
| AB026257 | 5006264 |
| AB026723 | 5931601 |
| AB027013 | 5931609 |
| AB027466 | 6172220 |
| AB028624 | 5103045 |
| AB028945 | 5689380 |
| AB028948 | 5689386 |
| AB028951 | 5689392 |
| AB028956 | 5689402 |
| AB028964 | 5689418 |
| AB028969 | 5689428 |
| AB028971 | 5689432 |
| AB028973 | 5689436 |
| AB028977 | 5689444 |
| AB028981 | 5689452 |
| AB028995 | 5689480 |
| AB029000 | 5689490 |
| AB029004 | 5689498 |
| AB029008 | 5689506 |
| AB029020 | 5689530 |
| AB029025 | 5689540 |
| AB029027 | 5689544 |
| AB029028 | 5689546 |
| AB029036 | 5689562 |
| AB029290 | 5821433 |
| AB032948 | 6329727 |
| AB032951 | 6329748 |
| AB032954 | 6382009 |
| AB032957 | 6329818 |
| AB032959 | 6329832 |
| AB032969 | 6329965 |
| AB032981 | 6330115 |
| AB032983 | 6330128 |
| AB032990 | 6330168 |
| AB032991 | 6330170 |
| AB032994 | 6330182 |
| AB033007 | 6330242 |
| AB033010 | 6330254 |
| AB033017 | 6330344 |
| AB033020 | 6330364 |
| AB033029 | 6330432 |
| AB033033 | 6330496 |
| AB033042 | 6330568 |
| AB033048 | 6330610 |
| AB033049 | 6330616 |
| AB033051 | 6330667 |
| AB033059 | 6330728 |
| AB033061 | 6330743 |
| AB033069 | 6330811 |
| AB033070 | 6330818 |
| AB033071 | 6330825 |
| AB033074 | 6330846 |
| AB033075 | 6330853 |
| AB033076 | 6330860 |
| AB033078 | 6330873 |
| AB033079 | 6382025 |
| AB033080 | 6330892 |
| AB033091 | 6331191 |
| AB033092 | 6331198 |
| AB033114 | 6331406 |
| AB033899 | 6174679 |
| AC000065 | 1669367 |
| AC000399 | 2133879 |
| AC002040 | 2347081 |
| AC002064 | 2076723 |
| AC002086 | 2085785 |
| AC003991 | 2772535 |
| AC004022 | 3598727 |
| AC004130 | 2842785 |
| AC004223 | 3253129 |
| AC004456 | 2979597 |
| AC004499 | 2996641 |
| AC004507 | 2996632 |
| AC004656 | 3253114 |
| AC004706 | 3176728 |
| AC004816 | 4156209 |
| AC004982 | 3419846 |
| AC005004 | 6624123 |
| AC005037 | 4827310 |
| AC005154 | 3242763 |
| AC005383 | 3818355 |
| AC005480 | 4835818 |
| AC005538 | 4508129 |
| AC005831 | 4165331 |
| AC005880 | 6249667 |
| AC005881 | 6382477 |
| AC006057 | 4731048 |
| AC006153 | 4309917 |
| AC006257 | 4092478 |
| AC006285 | 7109501 |
| AC007052 | 4510438 |
| AC007239 | 5523828 |
| AC007275 | 10801446 |
| AC007279 | 10440749 |
| AC007686 | 6466519 |
| AD001528 | 2198556 |
| AF000152 | 2454301 |
| AF000231 | 2149974 |
| AF000364 | 2697102 |
| AF000381 | 2565195 |
| AF000652 | 2795862 |
| AF000982 | 2580549 |
| AF000983 | 2580551 |
| AF000984 | 2580553 |
| AF000986 | 6007846 |
| AF000994 | 2580573 |
| AF001212 | 2150045 |
| AF001433 | 2529704 |
| AF001542 | 2529714 |
| AF001893 | 2529723 |
| AF001902 | 2078326 |
| AF002210 | 2431867 |
| AF002668 | 2232173 |
| AF002697 | 2511528 |
| AF002715 | 2352276 |
| AF003341 | 2183298 |
| AF003837 | 2228792 |
| AF004162 | 3046385 |
| AF004327 | 2257932 |
| AF004561 | 2209346 |
| AF004813 | 4877551 |
| AF005038 | 2232240 |
| AF005043 | 2213921 |
| AF005050 | 5902180 |
| AF006043 | 2674061 |
| AF006083 | 2282031 |
| AF006085 | 2282035 |
| AF006086 | 2282037 |
| AF006088 | 2282041 |
| AF006514 | 2645430 |
| AF007135 | 2852610 |
| AF007142 | 2852619 |
| AF007144 | 2852621 |
| AF007149 | 2852627 |
| AF007151 | 2852629 |
| AF007216 | 2281471 |
| AF007217 | 2253416 |
| AF007217 | 2253416 |
| AF007791 | 3779196 |
| AF008442 | 2266928 |
| AF008443 | 2266930 |
| AF008915 | 3093475 |
| AF009615 | 2393946 |
| AF009620 | 2429161 |
| AF010233 | 2895090 |
| AF010235 | 2707622 |
| AF010244 | 2612875 |
| AF010312 | 2415299 |
| AF010313 | 6468761 |
| AFO1O31S | 2415305 |
| AF011904 | 2282067 |
| AF012072 | 9967556 |
| AF012073 | 2735856 |
| AF012126 | 2832227 |
| AF012281 | 2944188 |
| AF012549 | 2317718 |
| AF012872 | 2326226 |
| AF013160 | 3540238 |
| AF013168 | 2331280 |
| AF013717 | 2992540 |
| AF013758 | 3046899 |
| AF013759 | 3153208 |
| AF013970 | 2801421 |
| AF013988 | 2318114 |
| AF014955 | 2772828 |
| AF015040 | 4102704 |
| AF015283 | 2384720 |
| AF015553 | 2415381 |
| AF016266 | 2529562 |
| AF016270 | 2655005 |
| AF016369 | 2708304 |
| AF016371 | 2708308 |
| AF016496 | 4102822 |
| AF016509 | 2338747 |
| AF017256 | 3642774 |
| AF017269 | 2394305 |
| AF017305 | 2656140 |
| AF017445 | 2582184 |
| AF017688 | 2435451 |
| AF017714 | 2435477 |
| AF017748 | 4102915 |
| AF017782 | 3986404 |
| AF019214 | 2460168 |
| AF019226 | 3406426 |
| AF019632 | 4699520 |
| AF020038 | 3641397 |
| AF020202 | 2431999 |
| AF020351 | 2655052 |
| AF020591 | 2843170 |
| AF020736 | 3450954 |
| AF020761 | 5733825 |
| AF020768 | 2460246 |
| AF020797 | 9256827 |
| AF021336 | 2541972 |
| AF021351 | 2460207 |
| AF021819 | 2460317 |
| AF022108 | 2736148 |
| AF022212 | 5731365 |
| AF022215 | 2583011 |
| AF022229 | 2809382 |
| AF022913 | 2558890 |
| AF023244 | 4103459 |
| AF023259 | 3746337 |
| AF023676 | 3211721 |
| AF025684 | 2589220 |
| AF025840 | 2697122 |
| AF025998 | 2570851 |
| AF026166 | 4090928 |
| AF026169 | 5091668 |
| AF026246 | 2587022 |
| AF026247 | 2587025 |
| AF026291 | 2559007 |
| AF026292 | 2559009 |
| AF026939 | 2612967 |
| AF026977 | 2583080 |
| AF027302 | 2522533 |
| AF027515 | 2772909 |
| AF027024 | 5669560 |
| AF028593 | 2599081 |
| AF028832 | 3287488 |
| AF029669 | 2909800 |
| AF029689 | 4545074 |
| AF029890 | 2745882 |
| AF030162 | 2599128 |
| AF030234 | 2822459 |
| AF030310 | 2613043 |
| AF030403 | 4103970 |
| AF031647 | 2688988 |
| AF032456 | 3004908 |
| AF032885 | 2895491 |
| AF032922 | 3820481 |
| AF033026 | 3378100 |
| AF033095 | 2645728 |
| AF034091 | 3834383 |
| AF034174 | 2707736 |
| AF034176 | 2707738 |
| AF034209 | 2724107 |
| AF034607 | 4426566 |
| AF034759 | 2653648 |
| AF034795 | 2853613 |
| AF035268 | 4090959 |
| AF035285 | 2661036 |
| AF035286 | 2661038 |
| AF035293 | 2661047 |
| AF035295 | 2661050 |
| AF035296 | 2661052 |
| AF035298 | 2661054 |
| AP035309 | 2661070 |
| AF035316 | 2661078 |
| AF035319 | 2661082 |
| AF035537 | 2665741 |
| AF035812 | 2665835 |
| AF035839 | 3098337 |
| AF037447 | 6466790 |
| AF038172 | 2795890 |
| AF038175 | 2795894 |
| AF038187 | 2795907 |
| AF038404 | 2707904 |
| AF038440 | 2773041 |
| AF038451 | 3779225 |
| AF038452 | 3779227 |
| AF038952 | 3329373 |
| AF038955 | 3329379 |
| AF038957 | 3329383 |
| AF038960 | 3329389 |
| AF038962 | 3329393 |
| AF038963 | 4405794 |
| AF039023 | 3064244 |
| AF039029 | 3834389 |
| AF039081 | 3135472 |
| AF039692 | 3170183 |
| AF039703 | 2789655 |
| AF039918 | 3335101 |
| AF040250 | 2827256 |
| AF040707 | 3688794 |
| AF040958 | 2773338 |
| AF041259 | 3335396 |
| AF041260 | 3335398 |
| AF041483 | 3493528 |
| AF042166 | 3298596 |
| AF042181 | 3688781 |
| AF042284 | 5256829 |
| AF042331 | 3882901 |
| AF042346 | 4104932 |
| AF042384 | 2828146 |
| AF042385 | 2828148 |
| AF042386 | 2828150 |
| AF044195 | 3757821 |
| AF044321 | 3170263 |
| AF044588 | 2865520 |
| AF044670 | 4191318 |
| AF044671 | 4105274 |
| AF044953 | 5326824 |
| AF044954 | 4164441 |
| AP044956 | 5326827 |
| AF044957 | 4164445 |
| AF045167 | 2865608 |
| AP045184 | 3417598 |
| AF045229 | 2906029 |
| AF045583 | 3372492 |
| AF046001 | 2895869 |
| AF046785 | 3219696 |
| AF047020 | 4204096 |
| AF047185 | 2909861 |
| AF047436 | 3335127 |
| AF047440 | 3335135 |
| AF047442 | 3335139 |
| AF047470 | 2906145 |
| AF047472 | 2921872 |
| AF047489 | 6288685 |
| AP047599 | 2906227 |
| AF048731 | 2981197 |
| AF048977 | 3005586 |
| AF049910 | 3435156 |
| AF050171 | 5668577 |
| AF050637 | 4164451 |
| AF050638 | 5326819 |
| AF050641 | 5326822 |
| AF051321 | 4091773 |
| AF051323 | 4091777 |
| AF051334 | 3126794 |
| AF051941 | 3893858 |
| AF052093 | 3360399 |
| AF052097 | 3360404 |
| AF052107 | 3360414 |
| AF052113 | 3360420 |
| AF052124 | 3360431 |
| AF052129 | 3360438 |
| AF052138 | 3360447 |
| AF052149 | 3360459 |
| AF052153 | 3360463 |
| AF052154 | 3360465 |
| AF052159 | 3360470 |
| AF052164 | 3360475 |
| AF052175 | 3360486 |
| AF052180 | 3360492 |
| AF052182 | 3360494 |
| AF052183 | 3360495 |
| AF052187 | 3360499 |
| AF052578 | 2967847 |
| AF052642 | 2981453 |
| AF053070 | 3095112 |
| AF053470 | 2982365 |
| AF053535 | 4007411 |
| AF053551 | 3283048 |
| AF053641 | 3560556 |
| AF053970 | 2996005 |
| AF053977 | 3283050 |
| AF054175 | 3341993 |
| AF054179 | 3341999 |
| AF054181 | 3342003 |
| AF054182 | 3342005 |
| AF054183 | 4092053 |
| AF054186 | 3342007 |
| AF054187 | 4092059 |
| AF054284 | 4033734 |
| AF054838 | 2997740 |
| AF054987 | 3005697 |
| AF054988 | 3005699 |
| AF054990 | 3005703 |
| AF055008 | 3005729 |
| AF055012 | 3005735 |
| AF055022 | 3005750 |
| AF055025 | 3005753 |
| AF055033 | 3005763 |
| AF055470 | 4321663 |
| AF055473 | |
| AF055474 | |
| AF055994 | 3319289 |
| AF056087 | 3033550 |
| AF056322 | 3252910 |
| AF056433 | 3044151 |
| AF056617 | 6002477 |
| AF057160 | 3694919 |
| AF057299 | 5739040 |
| AF057705 | 3047335 |
| AF057706 | 3047336 |
| AF058319 | 3789788 |
| AF058718 | 3808234 |
| AF058953 | 3766196 |
| AF059524 | 4091867 |
| AF060152 | 5725505 |
| AF060219 | 3789798 |
| AF058718 | 3746548 |
| AF061258 | 3108092 |
| AF061261 | 3779239 |
| AF061737 | 4335938 |
| AF061739 | 4335942 |
| AF061938 | 4335946 |
| AF062318 | 3152814 |
| AF062323 | 3152824 |
| AF062725 | 4731781 |
| AF062729 | 4731785 |
| AF063521 | 5080891 |
| AF063657 | 3132830 |
| AF064084 | 3135668 |
| AF064257 | 5881960 |
| AF064484 | 3211761 |
| AF064491 | 3372806 |
| AF064768 | 3551827 |
| AF064769 | 3551829 |
| AF064819 | 6137096 |
| AF064859 | 3171152 |
| AF065388 | 3152700 |
| AF065391 | 4191326 |
| AF067008 | 3873220 |
| AF067170 | 4894373 |
| AF067171 | 4894375 |
| AF067396 | 4138825 |
| AF067656 | 3901271 |
| AF067972 | 4927369 |
| AF068117 | 3201959 |
| AF068180 | 3406748 |
| AF068235 | 4321975 |
| AF068302 | 4584876 |
| AF068754 | 3283408 |
| AF068846 | 3201999 |
| AF069073 | 3202003 |
| AF069301 | 3193335 |
| AF069469 | 4106788 |
| AF069601 | 7239697 |
| AF069762 | 4995955 |
| AF069785 | 3243032 |
| AF070523 | 3764088 |
| AF070525 | 3387880 |
| AF070539 | 3387896 |
| AF070548 | 3387910 |
| AF070553 | 3387917 |
| AF070554 | 3387919 |
| AF070555 | 3387920 |
| AF070556 | 3387921 |
| AF070561 | 3387928 |
| AF070562 | 3387930 |
| AF070595 | 3387972 |
| AF070596 | 3387973 |
| AF070597 | 3387974 |
| AF070600 | 3387979 |
| AF070618 | 3283884 |
| AF070621 | 3283887 |
| AF070626 | 3283892 |
| AF070641 | 3283914 |
| AF070646 | 3283920 |
| AF070649 | 3283923 |
| AF070650 | 4454675 |
| AF070651 | 4454677 |
| AF070652 | 4454679 |
| AF070655 | 4454685 |
| AF070857 | 4454689 |
| AF070658 | 4454691 |
| AF070659 | 4454693 |
| AF070660 | 4454695 |
| AF070661 | 4454697 |
| AF070664 | 4454703 |
| AF070665 | 4454705 |
| AF070668 | 4454711 |
| AF071076 | 4545098 |
| AF071172 | 5107833 |
| AF071202 | 3335172 |
| AF071593 | 3249712 |
| AF071748 | 3916211 |
| AF072371 | 5305400 |
| AF072810 | 4049921 |
| AF072847 | 3283980 |
| AF073298 | 3641537 |
| AF073299 | 5305404 |
| AF073310 | 4511968 |
| AF073475 | 3293552 |
| AF073887 | 4406092 |
| AF074016 | 3328174 |
| AF074606 | 4091979 |
| AF075061 | 3377602 |
| AF075587 | 3319325 |
| AF076929 | 4836160 |
| AF077029 | 4689105 |
| AF077030 | 4689107 |
| AF077032 | 4689111 |
| AF077034 | 4689115 |
| AF077035 | 4689117 |
| AF077037 | 4689121 |
| AF077042 | 4689131 |
| AF077043 | 4689133 |
| AF077045 | 4689137 |
| AF077200 | 4679013 |
| AF077202 | 4679017 |
| AF077367 | 3820534 |
| AF077599 | 3342561 |
| AF078845 | 5531804 |
| AF078847 | 5531808 |
| AF078848 | 5531810 |
| AF078849 | 5531812 |
| AF078854 | 5531822 |
| AF078855 | 5531824 |
| AF078858 | 5531830 |
| AF078859 | 5531832 |
| AF078860 | 5531834 |
| AF078861 | 5531836 |
| AF078862 | 5531838 |
| AF078863 | 5531840 |
| AF078864 | 5531842 |
| AF081192 | 3420798 |
| AF081259 | 4558757 |
| AF081281 | 3415122 |
| AF081282 | 4336324 |
| AF082513 | 3649656 |
| AF082657 | 3415108 |
| AF082858 | 4587463 |
| AF082868 | 3746804 |
| AF082889 | 4249648 |
| AF083106 | 7555470 |
| AF083107 | 5225319 |
| AF083190 | 3599414 |
| AF083217 | 5639662 |
| AF083395 | 4106817 |
| AF083441 | 5813822 |
| AF083470 | 3719293 |
| AF084260 | 3514096 |
| AF084457 | 5257006 |
| AF084555 | 5813858 |
| AF085224 | 5817298 |
| AF085243 | 5901526 |
| AF085355 | 5114044 |
| AF085359 | 5114052 |
| AF085362 | 5114058 |
| AF085844 | 3483158 |
| AF085845 | 3483159 |
| AF085850 | 3483164 |
| AF085858 | 3483174 |
| AF085871 | 3483194 |
| AF085893 | 3483227 |
| AF085896 | 3483231 |
| AF085917 | 3483257 |
| AF086002 | 3483347 |
| AF086003 | 3483348 |
| AF086168 | 3483513 |
| AF086172 | 3483517 |
| AF086179 | 3483524 |
| AF086182 | 3483527 |
| AF086183 | 3483528 |
| AF086218 | 3483563 |
| AF086236 | 3483581 |
| AF086245 | 3483590 |
| AF086251 | 3483596 |
| AF086336 | 3483681 |
| AF086351 | 3483696 |
| AF086401 | 3483746 |
| AF086418 | 3483763 |
| AF086431 | 3483776 |
| AF086449 | 3483794 |
| AF086472 | 3483817 |
| AF086477 | 3483822 |
| AF086495 | 3483840 |
| AF086517 | 3483862 |
| AF086557 | 3483902 |
| AF087020 | 3851144 |
| AF087438 | 3820591 |
| AF087481 | 4322487 |
| AF087573 | 4926917 |
| AF087942 | 5814084 |
| AF087980 | 3523186 |
| AF087999 | 3523205 |
| AF088004 | 3523210 |
| AF088035 | 3523241 |
| AF088055 | 3523261 |
| AF089747 | 4165889 |
| AF090327 | 3643252 |
| AF091071 | 3859979 |
| AF091073 | 3859983 |
| AF091076 | 3859989 |
| AF091083 | 3860003 |
| AF091084 | 3860005 |
| AF091089 | 3860015 |
| AF091090 | 3860017 |
| AF091263 | 4140646 |
| AF092038 | 4588026 |
| AF092092 | 4426602 |
| AF092133 | 5138915 |
| AF092134 | 5133917 |
| AF092135 | 5138919 |
| AF092138 | 5138925 |
| AF092565 | 3661609 |
| AF092905 | 6010437 |
| AF093097 | 6002622 |
| AF093118 | 3676821 |
| AF093668 | 3907589 |
| AF095687 | 4028562 |
| AF095770 | 4588084 |
| AF096773 | 5852345 |
| AF096774 | 5852347 |
| AF097159 | 5305556 |
| AF097514 | 4808600 |
| AF097535 | 6165619 |
| AF097725 | 4323032 |
| AF098297 | 4160442 |
| AF098533 | 4050041 |
| AF098865 | 4204674 |
| AF098951 | 4038351 |
| AF099137 | 4566496 |
| AF099149 | 3930775 |
| AF099989 | 3851170 |
| AF100615 | 4808624 |
| AF100741 | 5138992 |
| AF100742 | 5138996 |
| AF100744 | 5410273 |
| AF100749 | 5410283 |
| AF100755 | 5410295 |
| AF100756 | 5410297 |
| AF100757 | 5410299 |
| AF100759 | 5410303 |
| AF100761 | 5410307 |
| AF101044 | 4877591 |
| AF101051 | 4323580 |
| AF101074 | 4249704 |
| AF102265 | 3851710 |
| AF102846 | 10257493 |
| AF102850 | 5281120 |
| AF102988 | 5305593 |
| AF103720 | 4324281 |
| AF103774 | 6179867 |
| AF103801 | 6048969 |
| AF103804 | 6048975 |
| AF103907 | 6165973 |
| AF103908 | 6165974 |
| AF104012 | 4406226 |
| AF104013 | 4406228 |
| AF104252 | 4220891 |
| AF104914 | 4206125 |
| AF106681 | 5410327 |
| AF106862 | 5757683 |
| AF106943 | 4454994 |
| AF107405 | 5531903 |
| AP108092 | 4691548 |
| AF109219 | 4206154 |
| AF110377 | 4151928 |
| AF110643 | 5730475 |
| AF110647 | 5730483 |
| AF111106 | 4191593 |
| AF111713 | 5326796 |
| AF112152 | 5305672 |
| AF112227 | 4545218 |
| AF112481 | 4959395 |
| AF112972 | 4151943 |
| AF113131 | 4455014 |
| AF113132 | 5326801 |
| AF113140 | 4512029 |
| AF114027 | 5649915 |
| AF114471 | 4378758 |
| AF114816 | 5059036 |
| AF114817 | 5059038 |
| AF115402 | 4559272 |
| AF116827 | 4768830 |
| AF117754 | 4530436 |
| AF117756 | 4530440 |
| AF117949 | 4959424 |
| AF118124 | 4235636 |
| AF118838 | 5052318 |
| AF119121 | 6724278 |
| AF119297 | 4633508 |
| AF124141 | 4757578 |
| AF124438 | 4838431 |
| AF124439 | 4838433 |
| AF124440 | 4877758 |
| AF124598 | 4884701 |
| AF125096 | 5106987 |
| AF125097 | 5106989 |
| AF125100 | 5106995 |
| AF125392 | 5410354 |
| AF125393 | 5410356 |
| AF126008 | 4469557 |
| AF126181 | 4732088 |
| AF126245 | 6465954 |
| AF126424 | 4325326 |
| AF126736 | 4454564 |
| AF126780 | 6318543 |
| AF126782 | 6318547 |
| AF128406 | 5777951 |
| AF128527 | 4928043 |
| AF128528 | 4928045 |
| AF129927 | 4558506 |
| AF131220 | 4589247 |
| AF131738 | 4406548 |
| AF131743 | 4406555 |
| AF131749 | 4406565 |
| AF131758 | 4406576 |
| AF131763 | 4406585 |
| AF131774 | 4406598 |
| AF131775 | 4406599 |
| AF131784 | 4406612 |
| AF131792 | 4406620 |
| AF131802 | 4406633 |
| AF131814 | 4406648 |
| AF131831 | 4406669 |
| AF131838 | 4406677 |
| AF131844 | 4406685 |
| AF131856 | 4406702 |
| AF131857 | 4406704 |
| AF132000 | 4530586 |
| AF132937 | 4680644 |
| AF132940 | 4680650 |
| AF132941 | 4680652 |
| AF132945 | 4680660 |
| AF132956 | 4680682 |
| AF132965 | 4680700 |
| AF132968 | 4680706 |
| AF133123 | 5281313 |
| AF133845 | 4884871 |
| AF134159 | 5870835 |
| AF135421 | 5052350 |
| AF135488 | 5668619 |
| AF136450 | 4835894 |
| AF137334 | 4583504 |
| AF138300 | 5532410 |
| AF139461 | 4894945 |
| AF140598 | 4769003 |
| AF141327 | 4769007 |
| AF141968 | 6273777 |
| AF143235 | 6449465 |
| AF143886 | 4895031 |
| AF144103 | 5059165 |
| AF144566 | 4809247 |
| AF144755 | 5006628 |
| AF145029 | 5052413 |
| AF145316 | 4929324 |
| AF146277 | 4960046 |
| AF147330 | 4761681 |
| AF147331 | 4761682 |
| AF147334 | 4761685 |
| AF147339 | 4761690 |
| AF147354 | 4761705 |
| AF147367 | 4761718 |
| AF147380 | 4761731 |
| AF147395 | 4761746 |
| AF149045 | 6010479 |
| AF150087 | 5107162 |
| AF150105 | 5107197 |
| AF150208 | 5133644 |
| AF151103 | 5758136 |
| AF151105 | 5739542 |
| AF151109 | 6166337 |
| AF151793 | 6424941 |
| AF151800 | 4929552 |
| AF151803 | 4929558 |
| AF151805 | 4929562 |
| AF151818 | 4929588 |
| AF151820 | 4929592 |
| AF151823 | 4929598 |
| AF151830 | 4929612 |
| AF151836 | 4929624 |
| AF151837 | 4929626 |
| AF151840 | 4929632 |
| AF151844 | 4929640 |
| AF151846 | 4929644 |
| AF151851 | 4929654 |
| AF151855 | 4929662 |
| AF151859 | 4929670 |
| AF151861 | 4929674 |
| AF151867 | 4929686 |
| AF151868 | 4929688 |
| AF151870 | 4929692 |
| AF151871 | 4929694 |
| AF151873 | 4929698 |
| AF151875 | 4929702 |
| AF151878 | 4929708 |
| AF151884 | 4929720 |
| AF151893 | 4929738 |
| AF151896 | 4929744 |
| AF151897 | 4929746 |
| AF151902 | 4929756 |
| AF151903 | 4929758 |
| AF151906 | 4929764 |
| AF151908 | 4929788 |
| AF152097 | 4929772 |
| AF152306 | 5456897 |
| AF153191 | 7145110 |
| AF153201 | 5020361 |
| AF153603 | 5231130 |
| AF153608 | 5231140 |
| AF153612 | 4929830 |
| AF155095 | 5360084 |
| AF155099 | 5360092 |
| AF155160 | 5360094 |
| AF155110 | 5360114 |
| AF155113 | 5360120 |
| AF155235 | 6318598 |
| AF155330 | 5107389 |
| AF155568 | 5031511 |
| AF155832 | 6288768 |
| AF156098 | 5070697 |
| AF156965 | 5731112 |
| AF156972 | 5361367 |
| AF157028 | 5533002 |
| AF159056 | 5566238 |
| AF159092 | 9055139 |
| AF159295 | 5714635 |
| AF159615 | 6466003 |
| AF165281 | 5734100 |
| AF167160 | 5733691 |
| AF167572 | 6164703 |
| AF168956 | 5702387 |
| AF169481 | 6272653 |
| AF169797 | 6118085 |
| AF170583 | 6318677 |
| AF170708 | 6073867 |
| AF171877 | 6002954 |
| AF171944 | 5802476 |
| AF174595 | 6164732 |
| AF174605 | 6164752 |
| AF176555 | 6318854 |
| AF176574 | 5762481 |
| AF176642 | 6935100 |
| AF176700 | 6103638 |
| AF176702 | 6103642 |
| AF177291 | 5882256 |
| AF177775 | 5823471 |
| AF178030 | 6539738 |
| AF179274 | 5911402 |
| AF182289 | 5919146 |
| AF182645 | 5901877 |
| AF188298 | 6176335 |
| AF188745 | 6425043 |
| AF188746 | 6425045 |
| AF188747 | 6425047 |
| AF190167 | 6456117 |
| AF191018 | 6457339 |
| AF191020 | 6457343 |
| AF191298 | 7656642 |
| AF191339 | 6180012 |
| AF192979 | 6409378 |
| AF193795 | 6164954 |
| AF195120 | 6110609 |
| AF195417 | 6118554 |
| AF198358 | 6466185 |
| AF201077 | 6456748 |
| AI000271 | 3190825 |
| AI000294 | 3190848 |
| AI000502 | 3191056 |
| AI000877 | 3191431 |
| AI001862 | 3202333 |
| AI002321 | 3202655 |
| AI002745 | 3203159 |
| AI002821 | 3203235 |
| AI003028 | 3203442 |
| AI003625 | 3203959 |
| AI003636 | 3203970 |
| AI003778 | 3213288 |
| AI003782 | 3213292 |
| AI003893 | 3213403 |
| AI004135 | 3213645 |
| AI004169 | 3213679 |
| AI004252 | 3213762 |
| AI004315 | 3213825 |
| AI004596 | 3214106 |
| AI004916 | 3214426 |
| AI004930 | 3214440 |
| AI005274 | 3214784 |
| AI005321 | 3214831 |
| AI005449 | 3214959 |
| AI005515 | 3215025 |
| AI005519 | 3215029 |
| AI00552l | 3215031 |
| AI014387 | 3228768 |
| AI014704 | 3229085 |
| AI015053 | 3229389 |
| AI015196 | 3229532 |
| AI015245 | 3229581 |
| AI015359 | 3229695 |
| AI015574 | 3229910 |
| AI015589 | 3229925 |
| AI015679 | 3230015 |
| AI015790 | 3230126 |
| AI01603l | 3230367 |
| A1016074 | 3230410 |
| AI016106 | 3230442 |
| AI016113 | 3230449 |
| AI016162 | 3230498 |
| AI016169 | 3230505 |
| AI016417 | 3230753 |
| AI016618 | 3230954 |
| AI016688 | 3231024 |
| AI016700 | 3231036 |
| AI017022 | 3231358 |
| AI017236 | 3231572 |
| AI017239 | 3231575 |
| AI017240 | 3231576 |
| AI017332 | 3231668 |
| AI017442 | 3231778 |
| AI017607 | 3231943 |
| AI017703 | 3232039 |
| AI017779 | 3232115 |
| AI017809 | 3232145 |
| AI017959 | 3232295 |
| AI017996 | 3232332 |
| AI018034 | 3232370 |
| AI018161 | 3232680 |
| AI018468 | 3232987 |
| AI018578 | 3233097 |
| AI018613 | 3233132 |
| AI018617 | 3233136 |
| AI018625 | 3233144 |
| AI018686 | 3232484 |
| AI018728 | 3232526 |
| AI018773 | 3232571 |
| AI021925 | 3237538 |
| AI022058 | 3239411 |
| AI022134 | 3239487 |
| AI022340 | 3237581 |
| AI022353 | 3237594 |
| AI022687 | 3237928 |
| AI022855 | 3238096 |
| AI023483 | 3238527 |
| AI023724 | 3238768 |
| AI023770 | 3238814 |
| AI023931 | 3238975 |
| AI024401 | 3240014 |
| AI024549 | 3240162 |
| AI024638 | 3240251 |
| AI024799 | 3240412 |
| AI024907 | 3240520 |
| AI025015 | 3240628 |
| AI1025120 | 3240733 |
| AI025126 | 3240739 |
| AI025259 | 3240872 |
| AI025262 | 3240875 |
| AI025376 | 3240989 |
| AI025419 | 3241032 |
| AI025519 | 3241132 |
| AI026122 | 3241735 |
| AI026767 | 3246255 |
| AI026819 | 3246307 |
| AI027232 | 3244748 |
| AI027516 | 3246446 |
| AI027536 | 3246466 |
| AI027643 | 3245082 |
| AI027887 | 3246586 |
| AI028097 | 3245406 |
| AI028107 | 3245416 |
| AI028122 | 3245431 |
| AI028507 | 3245816 |
| AI028516 | 3245825 |
| AI028708 | 3246017 |
| AI031653 | 3249865 |
| AI031665 | 3249877 |
| AI031819 | 3250031 |
| AI032392 | 3250604 |
| AI032401 | 3250613 |
| AI032759 | 3253456 |
| AI032779 | 3253476 |
| AI032838 | 3253535 |
| AI033863 | 3254816 |
| AI033904 | 3254857 |
| AI033912 | 3254865 |
| AI034024 | 3254977 |
| AI034131 | 3255084 |
| AI034431 | 3255384 |
| AI038038 | 3277232 |
| AI038048 | 3277242 |
| AI038167 | 3277361 |
| AI038415 | 3277609 |
| AI038692 | 3277886 |
| AI038986 | 3278180 |
| AI039200 | 3278394 |
| AI039227 | 3278421 |
| AI039271 | 3278465 |
| AI039301 | 3278495 |
| AI039433 | 3278627 |
| AI039636 | 3278830 |
| AI039804 | 3278998 |
| AI039813 | 3279007 |
| AI040234 | 3279428 |
| AI040339 | 3279533 |
| AI040598 | 3279792 |
| AI041116 | 3280310 |
| AI041150 | 3280344 |
| AI041497 | 3280691 |
| AI041616 | 3280810 |
| AI041630 | 3280824 |
| AI041820 | 3281014 |
| AI041847 | 3281041 |
| AI042002 | 3281196 |
| AI042059 | 3281253 |
| AI042066 | 3281260 |
| AI042152 | 3281346 |
| AI042153 | 3281347 |
| AI042290 | 3281484 |
| AI042293 | 3281487 |
| AI042354 | 3281548 |
| AI042406 | 3281600 |
| AI049971 | 3299088 |
| AI049995 | 3299112 |
| AI050826 | 3307631 |
| AI050871 | 3307676 |
| AI050872 | 3307677 |
| AI050966 | 3307771 |
| AI051210 | 3306744 |
| AI051376 | 3306910 |
| AI051957 | 3307948 |
| AI051959 | 3307950 |
| AI051983 | 3307974 |
| AI051987 | 3307978 |
| AI051994 | 3307985 |
| AI052061 | 3308052 |
| AI052112 | 3308103 |
| AI052124 | 3308115 |
| AI052126 | 3308117 |
| AI052333 | 3308324 |
| AI052453 | 3308444 |
| AI052525 | 3308516 |
| AI052724 | 3308715 |
| AI052738 | 3308729 |
| AI053597 | 3321384 |
| AI053744 | 3321531 |
| AI054023 | 3321810 |
| AI054344 | 3322131 |
| AI055870 | 3329736 |
| AI055939 | 3329805 |
| AI056489 | 3330355 |
| AI056927 | 3330716 |
| AI057093 | 3330969 |
| AI057142 | 3331008 |
| AI057281 | 3331147 |
| AI057555 | 3331421 |
| AI057585 | 3331451 |
| AI057617 | 3331483 |
| AI061116 | 3336484 |
| AI061420 | 3336788 |
| AI061610 | 6358907 |
| AI061649 | 6358946 |
| AI064691 | 6358963 |
| AI064763 | 6359035 |
| AI064844 | 6359116 |
| AI064931 | 6359203 |
| AI064967 | 6359239 |
| AI065092 | 6359364 |
| AI065140 | 6359412 |
| AI065152 | 6359424 |
| AI066442 | 3367144 |
| AI066518 | 3367220 |
| AI066753 | 3367039 |
| AI073465 | 3400109 |
| AI073907 | 3400551 |
| AI074107 | 3400751 |
| AI074143 | 3400787 |
| AI074232 | 3400876 |
| AI074308 | 3400952 |
| AI074389 | 3401033 |
| AI074397 | 3401041 |
| AI074613 | 3401257 |
| AI074652 | 3401296 |
| AI074713 | 3401357 |
| AI074969 | 3401613 |
| AI075037 | 3401681 |
| AI075041 | 3401685 |
| AI075189 | 3401780 |
| AI075211 | 3401802 |
| AI075324 | 3399895 |
| AI075338 | 3399909 |
| AI075631 | 3404809 |
| AI075893 | 3405071 |
| AI075935 | 3405113 |
| AI075963 | 3405141 |
| AI076042 | 3405220 |
| AI076593 | 3405771 |
| AI076608 | 3405786 |
| AI077317 | 3411725 |
| AI077439 | 3411847 |
| AI077488 | 3411896 |
| AI077500 | 3411908 |
| AI077641 | 3412049 |
| AI077719 | 3412127 |
| AI078217 | 3412625 |
| AI078381 | 3412789 |
| AI078772 | 3413079 |
| AI078802 | 3413109 |
| AI079329 | 3415580 |
| AI079558 | 3415809 |
| AI079595 | 3415846 |
| AI079882 | 3416133 |
| AI080170 | 3416421 |
| AI080267 | 3416518 |
| AI080272 | 3416523 |
| AI080377 | 3416628 |
| AI080439 | 3416690 |
| AI080476 | 3416727 |
| AI080480 | 3416731 |
| AI080485 | 3416736 |
| AI081133 | 3417925 |
| AI081291 | 3418083 |
| AI081379 | 3418171 |
| AI081842 | 3418634 |
| AI081918 | 3418710 |
| AI081945 | 3418737 |
| AI082168 | 3418960 |
| AI082235 | 3419027 |
| AI082272 | 3419064 |
| AI082307 | 3419099 |
| AI082617 | 3419409 |
| AI082674 | 3419466 |
| AI082864 | 3417840 |
| AI083814 | 3422255 |
| AI084005 | 3422428 |
| AI084100 | 3422523 |
| AI084101 | 3422524 |
| AI084457 | 3422880 |
| AI084466 | 3422889 |
| AI084604 | 3423027 |
| AI084651 | 3423074 |
| AI084916 | 3423339 |
| AI085165 | 3423588 |
| AI085195 | 3423618 |
| AI085263 | 3423686 |
| AI085381 | 3423804 |
| AI085393 | 3423816 |
| AI085554 | 3423977 |
| AI085559 | 3423982 |
| AI085977 | 3424400 |
| AI086059 | 3424482 |
| AI086377 | 3424800 |
| AI087077 | 3425500 |
| AI087080 | 3425503 |
| AI087145 | 3425568 |
| AI087287 | 3425710 |
| AI087300 | 3425723 |
| AI087412 | 3425835 |
| AI087888 | 3426921 |
| AI087987 | 3427020 |
| AI088178 | 3427256 |
| AI088201 | 3427279 |
| AI088633 | 3427692 |
| AI088691 | 3427750 |
| AI088778 | 3427837 |
| AI088914 | 3427973 |
| AI089394 | 3428453 |
| AI089431 | 3428490 |
| AI089525 | 3428584 |
| AI089526 | 3428585 |
| AI089875 | 3428934 |
| AI089923 | 3428982 |
| AI089948 | 3429007 |
| AI090012 | 3429071 |
| AI090226 | 3429285 |
| AI090623 | 3429682 |
| AI090642 | 3429701 |
| AI090786 | 3429845 |
| AI090809 | 3429868 |
| AI090827 | 3429886 |
| AI091136 | 3430195 |
| AI091190 | 3430249 |
| AI091425 | 3430484 |
| AI091516 | 3430575 |
| AI091736 | 3430795 |
| AI091814 | 3430873 |
| AI091935 | 3430994 |
| AI092428 | 3431404 |
| AI092479 | 3431455 |
| AI092766 | 3431742 |
| AI093233 | 3432209 |
| AI093387 | 3432363 |
| AI093409 | 3432385 |
| AI093458 | 3432434 |
| AI093553 | 3432529 |
| AI093823 | 3432799 |
| AI094010 | 3432986 |
| AI094162 | 3433138 |
| AI094394 | 3433370 |
| AI094634 | 3433610 |
| AI094741 | 3433717 |
| AI094803 | 3433779 |
| AI095031 | 3434007 |
| AI095250 | 3434226 |
| AI095875 | 3434851 |
| AI096410 | 3446321 |
| AI096493 | 3445987 |
| AI096706 | 3446200 |
| A1097214 | 3446796 |
| A1097410 | 3446992 |
| A1097545 | 3447127 |
| AI110765 | 6359630 |
| AI110836 | 6359699 |
| AI110856 | 6359719 |
| AI110866 | 6359729 |
| AI114433 | 6359778 |
| AI114467 | 6359812 |
| AI114489 | 6359834 |
| AI114602 | 6359947 |
| AI114631 | 6359976 |
| AI114651 | 6359996 |
| AI123199 | 3538965 |
| AI123229 | 3538995 |
| AI123347 | 3539113 |
| AI123672 | 3539438 |
| AI123822 | 3539588 |
| AI123916 | 3539682 |
| AI124532 | 3593046 |
| AI124856 | 3593370 |
| AI125166 | 3593680 |
| AI125607 | 3594121 |
| AI125642 | 3594156 |
| AI125653 | 3594167 |
| AI125688 | 3594202 |
| AI126093 | 3594607 |
| AI126233 | 3594747 |
| AI126257 | 3594771 |
| AI126653 | 3595167 |
| AI126700 | 3595214 |
| AI126867 | 3595381 |
| AI127167 | 3595681 |
| AI127176 | 3595690 |
| AI127239 | 3595753 |
| AI127268 | 3595782 |
| AI127424 | 3595938 |
| AI127455 | 3595969 |
| AI127526 | 3596040 |
| AI127556 | 3596070 |
| AI127923 | 3596437 |
| AI128091 | 3596605 |
| AI128828 | 3597342 |
| AI128883 | 3597397 |
| AI128918 | 3597432 |
| AI128949 | 3597463 |
| AI128986 | 3597500 |
| AI129280 | 3597782 |
| AI129321 | 3597835 |
| AI129342 | 3597856 |
| AI129403 | 3597917 |
| AI129714 | 3598228 |
| AI129800 | 3598314 |
| AI129891 | 3598405 |
| AI129932 | 3598446 |
| AI129991 | 3598505 |
| AI130796 | 3600812 |
| AI131097 | 3601113 |
| AI131216 | 3601232 |
| AI131444 | 3601460 |
| AI133096 | 6360412 |
| AI133138 | 6360454 |
| AI133152 | 6360468 |
| AI133208 | 6360524 |
| AI133247 | 6360563 |
| AI133330 | 6360646 |
| AI133381 | 6360697 |
| AI133387 | 6360703 |
| AI133402 | 6360718 |
| AI133406 | 6360722 |
| AI133466 | 6360782 |
| AI133562 | 6360878 |
| AI133612 | 6360928 |
| AI133668 | 6360984 |
| AI133690 | 6361006 |
| AI133727 | 3602925 |
| AI138629 | 3644601 |
| AI138633 | 3644605 |
| AI138640 | 3644612 |
| AI139036 | 3645008 |
| AI139157 | 3645129 |
| AI139200 | 3645172 |
| AI139394 | 3645366 |
| AI139904 | 3645876 |
| AI140096 | 3647553 |
| AI140210 | 3647667 |
| AI140251 | 3647708 |
| AI140421 | 3647878 |
| AI140793 | 3648250 |
| AI140921 | 3648378 |
| AI141040 | 3648497 |
| AI141189 | 3648646 |
| AI141483 | 3648940 |
| AI141583 | 3649040 |
| AI141732 | 3649189 |
| AI141770 | 3649227 |
| AI141847 | 3649304 |
| AI142021 | 3649478 |
| AI142083 | 3649540 |
| AI142095 | 3649552 |
| AI142257 | 3658616 |
| AI142558 | 3658917 |
| AI143199 | 3665008 |
| AI143623 | 3665432 |
| AI143746 | 3665555 |
| AI143775 | 3665584 |
| AI143899 | 3665708 |
| AI144216 | 3666025 |
| AI144378 | 3666187 |
| AI144423 | 3666232 |
| AI144488 | 3666297 |
| AI146340 | 3674022 |
| AI146476 | 3674158 |
| AI146478 | 3674160 |
| AI147191 | 3674873 |
| AI147472 | 3675154 |
| AI147850 | 3675532 |
| AI148115 | 3675797 |
| AI148251 | 3675933 |
| AI148392 | 3676861 |
| AI148437 | 3676906 |
| AI148552 | 3677021 |
| AI148561 | 3677030 |
| AI148676 | 3677145 |
| AI148933 | 3677402 |
| AI149363 | 3677832 |
| AI149592 | 3678061 |
| AI149743 | 3678212 |
| AI150619 | 3679088 |
| AI151140 | 3679609 |
| AI151458 | 3679927 |
| AI151482 | 3679951 |
| AI151497 | 3679966 |
| AI159768 | 3693127 |
| AI160324 | 3693704 |
| AI160426 | 3693806 |
| AI161068 | 3694373 |
| AI161253 | 3692941 |
| AI161261 | 3692949 |
| AI161272 | 3692960 |
| AI161333 | 3694577 |
| AI161350 | 3694594 |
| AI167330 | 3700500 |
| AI167651 | 3700821 |
| AI167801 | 3700971 |
| AI168205 | 3701375 |
| AI174394 | 3721247 |
| AI174675 | 6361053 |
| AI174822 | 6361214 |
| AI174828 | 6361222 |
| AI174853 | 6361253 |
| AI174867 | 6361268 |
| AI174899 | 6361302 |
| AI174957 | 6361365 |
| AI174991 | 6361399 |
| AI183358 | 3733996 |
| AI183447 | 3734085 |
| AI183745 | 3734383 |
| AI183897 | 3734535 |
| AI184113 | 3734751 |
| AI184320 | 3734958 |
| AI184562 | 3735200 |
| AI184748 | 3735386 |
| AI184994 | 3735632 |
| AI186545 | 3737183 |
| AI186597 | 3737235 |
| AI188078 | 3739287 |
| AI188081 | 3739290 |
| AI188563 | 3739772 |
| AI188641 | 3739850 |
| AI188794 | 3740003 |
| AI188850 | 3740059 |
| AI189173 | 3740382 |
| AI189339 | 3740548 |
| AI189340 | 3740549 |
| AI189345 | 3740554 |
| AI189386 | 3740595 |
| AI189841 | 3741050 |
| AI189894 | 3741103 |
| AI189897 | 3741106 |
| AI190480 | 3741689 |
| AI190648 | 3741857 |
| AI190785 | 3741994 |
| AI191244 | 3742453 |
| AI192767 | 3743976 |
| AI192780 | 3743989 |
| AI192784 | 3743993 |
| AI192932 | 3744141 |
| AI193675 | 3744872 |
| AI197946 | 3750552 |
| AI198311 | 3750917 |
| AI198317 | 3750923 |
| AI198416 | 3751022 |
| AI198577 | 3751183 |
| AI198625 | 3751231 |
| AI198771 | 3751377 |
| AI198853 | 3751459 |
| AI198859 | 3751465 |
| AI198930 | 3751536 |
| AI198937 | 3751543 |
| AI198986 | 3751592 |
| AI199094 | 3751700 |
| AI199544 | 3752150 |
| AI199641 | 3752247 |
| AI199681 | 3752287 |
| AI199773 | 3752379 |
| AI200038 | 3752644 |
| AI200110 | 3752716 |
| AI200245 | 3752851 |
| AI200324 | 3752930 |
| AI200444 | 3753050 |
| AI200578 | 3753184 |
| AI200798 | 3753404 |
| AI200830 | 3753436 |
| AI201248 | 3753854 |
| AI201261 | 3753867 |
| AI201294 | 3753900 |
| AI201298 | 3753904 |
| AI201483 | 3754089 |
| AI201484 | 3754090 |
| AI201564 | 3754170 |
| AI202026 | 3754632 |
| AI203141 | 3755747 |
| AI203350 | 3755956 |
| AI203377 | 3755983 |
| AI203395 | 3756001 |
| AI203546 | 3756152 |
| AI204090 | 3756696 |
| AI204096 | 3756702 |
| AI204308 | 3756914 |
| AI204916 | 3757978 |
| AI205228 | 3758290 |
| AI205868 | 3764540 |
| AI206063 | 3764735 |
| AI206199 | 3764871 |
| AI206223 | 3764895 |
| AI206344 | 3765016 |
| AI206483 | 3765155 |
| AI206818 | 3765490 |
| AI207367 | 3766039 |
| AI207515 | 6361523 |
| AI207528 | 6361536 |
| AI207546 | 6361554 |
| AI207570 | 6361578 |
| AI207618 | 6361632 |
| AI207628 | 6361642 |
| AI207650 | 6361664 |
| AI207660 | 6361674 |
| AI207781 | 3769723 |
| AI208430 | 3770372 |
| AI208947 | 3770889 |
| AI214043 | 3777644 |
| AI214048 | 3777649 |
| AI214050 | 3777651 |
| AI214054 | 3777655 |
| AI214272 | 3777873 |
| AI214784 | 3778385 |
| AI215841 | 3784882 |
| AI216138 | 3785179 |
| AI216358 | 3785399 |
| AI216966 | 3789620 |
| AI216968 | 3789622 |
| AI216969 | 3789623 |
| AI216971 | 3789625 |
| AI216972 | 3789626 |
| AI216979 | 3789633 |
| AI216984 | 3789638 |
| AI216986 | 3789640 |
| AI216988 | 3789642 |
| AI217003 | 3789657 |
| AI217009 | 3789663 |
| AI217012 | 3789666 |
| AI217015 | 3789669 |
| AI217019 | 3789673 |
| AI217021 | 3789675 |
| AI217023 | 3789677 |
| AI217172 | 3796987 |
| AI217425 | 3797240 |
| AI217557 | 3797372 |
| AI217694 | 3797509 |
| AI217964 | 3797779 |
| AI218376 | 3798191 |
| AI218882 | 3801085 |
| AI219116 | 3801319 |
| AI219174 | 3801377 |
| AI219278 | 3801481 |
| AI219324 | 3801527 |
| AI219570 | 3801773 |
| AI219803 | 3802006 |
| AI220051 | 3802254 |
| AI220149 | 3802352 |
| AI220151 | 3802354 |
| AI221390 | 3803593 |
| AI221713 | 3803916 |
| AI221829 | 3804032 |
| AI221894 | 3804097 |
| AI222329 | 3804532 |
| AI222506 | 3804709 |
| AI222789 | 3804992 |
| AI222806 | 3805009 |
| AI223061 | 3805264 |
| AI223085 | 3805288 |
| AI224519 | 3807232 |
| AI225043 | 3807756 |
| AI239435 | 3834832 |
| AI239506 | 3834903 |
| AI239822 | 3835219 |
| AI240543 | 3835940 |
| AI240872 | 3836269 |
| AI240899 | 3836296 |
| AI241117 | 3836514 |
| AI241212 | 3836609 |
| AI241659 | 3837056 |
| AI241744 | 3837141 |
| AI241893 | 3837290 |
| AI242701 | 3838098 |
| AI242761 | 3838158 |
| AI243343 | 3838740 |
| AI243423 | 3838820 |
| AI243780 | 3839177 |
| AI243841 | 3839238 |
| AI244140 | 3839537 |
| AI244217 | 3839614 |
| AI244335 | 3839732 |
| AI245297 | 3840694 |
| AI245327 | 3840724 |
| AI246167 | 3841564 |
| AI246173 | 3841570 |
| AI246383 | 3841780 |
| AI246497 | 3841894 |
| AI246615 | 3842012 |
| AI247078 | 3842475 |
| AI248187 | 3843584 |
| AI248277 | 3843674 |
| AI248514 | 3843911 |
| AI248721 | 3844118 |
| AI248736 | 3844133 |
| AI249000 | 3844397 |
| AI249257 | 3845786 |
| AI249680 | 3846209 |
| AI249764 | 3846293 |
| AI249797 | 3846326 |
| AI249866 | 3846395 |
| AI249877 | 3846406 |
| AI250055 | 3846584 |
| AI250092 | 3846621 |
| AI252084 | 3848613 |
| AI252283 | 3848812 |
| AI252562 | 3849091 |
| AI253099 | 3849628 |
| AI253192 | 3849721 |
| AI253288 | 3850409 |
| AI253293 | 3850414 |
| AI253300 | 3850421 |
| AI253304 | 3850425 |
| AI253319 | 3850440 |
| AI253330 | 3850451 |
| AI253335 | 3850456 |
| AI253338 | 3850459 |
| AI253345 | 3850466 |
| AI253347 | 3850468 |
| AI253348 | 3850469 |
| AI253363 | 3850484 |
| AI253367 | 3850488 |
| AI253379 | 3850500 |
| AI253381 | 3850502 |
| AI253395 | 3850350 |
| AI253424 | 3850379 |
| AI253436 | 3850391 |
| AI261763 | 3869966 |
| AI261883 | 3870086 |
| AI261899 | 3870102 |
| AI262104 | 3870307 |
| AI262139 | 3870342 |
| AI262460 | 3870663 |
| AI262486 | 3870689 |
| AI263110 | 3871313 |
| AI263731 | 3871934 |
| AI263876 | 3872079 |
| AI265776 | 3883934 |
| AI265881 | 3884039 |
| AI266043 | 3884201 |
| AI266643 | 3884801 |
| AI267158 | 3886325 |
| AI267162 | 3886329 |
| AI267185 | 3886352 |
| AI267216 | 3886383 |
| AI267254 | 3886421 |
| AI267255 | 3886422 |
| AI267256 | 3886423 |
| AI267257 | 3886424 |
| AI267271 | 3886438 |
| AI267276 | 3886443 |
| AI267282 | 3886449 |
| AI267285 | 3886452 |
| AI267307 | 3886474 |
| AI267321 | 3886488 |
| AI267335 | 3886502 |
| AI267353 | 3886520 |
| AI267384 | 3886551 |
| AI267397 | 3886564 |
| AI267416 | 3886583 |
| AI267417 | 3886584 |
| AI267442 | 3886609 |
| AI267454 | 3886621 |
| AI267461 | 3886628 |
| AI267480 | 3886647 |
| AI267490 | 3886657 |
| AI267492 | 3886659 |
| AI267502 | 3886669 |
| AI267519 | 3886686 |
| AI267522 | 3886689 |
| AI267554 | 3886721 |
| AI267570 | 3886737 |
| AI267574 | 3886741 |
| AI267596 | 3886763 |
| AI267612 | 3886779 |
| AI267652 | 3886819 |
| AI267656 | 3886823 |
| AI267664 | 3886831 |
| AI267669 | 3886836 |
| AI267677 | 3886844 |
| AI267808 | 3886975 |
| AI267838 | 3887005 |
| AI267845 | 3887012 |
| AI267849 | 3887016 |
| AI267941 | 3887108 |
| AI268328 | 3887495 |
| AI268362 | 3887529 |
| AI268405 | 3887572 |
| AI268430 | 3887597 |
| AI268724 | 3887891 |
| AI268860 | 3888027 |
| AI269053 | 3888220 |
| AI269060 | 3888227 |
| AI269158 | 3888325 |
| AI269205 | 3888372 |
| AI269415 | 3888582 |
| AI269747 | 3888914 |
| AI269883 | 3889050 |
| AI270511 | 3889678 |
| AI270536 | 3889703 |
| AI271269 | 3890436 |
| AI271740 | 3890907 |
| AI272941 | 3895209 |
| AI273636 | 3895904 |
| AI273945 | 3896213 |
| AI274700 | 3896974 |
| AI274786 | 3897060 |
| AI275114 | 3897388 |
| AI275175 | 3897449 |
| AI275711 | 3897985 |
| AI275792 | 3898066 |
| AI276322 | 3898596 |
| AI276341 | 3898615 |
| AI276399 | 3898673 |
| AI276576 | 3898850 |
| AI276662 | 3898936 |
| AI276705 | 3898979 |
| AI277434 | 3899702 |
| AI277739 | 3900007 |
| AI278014 | 3900282 |
| AI278611 | 3916845 |
| AI278643 | 3916877 |
| AI278659 | 3916893 |
| AI278838 | 3917072 |
| AI279141 | 3917375 |
| AI279838 | 3918072 |
| AI279882 | 3918116 |
| AI280561 | 3918794 |
| AI280637 | 3918870 |
| AI280935 | 3919168 |
| AI281196 | 3919429 |
| AI281449 | 3919682 |
| AI281509 | 3919742 |
| AI281762 | 3919995 |
| AI281777 | 3920010 |
| AI281882 | 3920115 |
| AI281926 | 3920159 |
| AI281946 | 3920179 |
| AI282277 | 3920510 |
| AI282318 | 3920551 |
| AI283096 | 3921329 |
| AI283385 | 3921618 |
| AI283489 | 3921722 |
| AI284011 | 3922244 |
| AI284549 | 3922782 |
| AI284667 | 3922900 |
| AI285408 | 3923641 |
| AI285435 | 3923668 |
| AI285488 | 3923721 |
| AI287603 | 3927356 |
| AI287972 | 3927725 |
| AI288142 | 3930919 |
| AI288388 | 3931068 |
| AI288730 | 3932233 |
| AI288929 | 3931238 |
| AI289059 | 3931368 |
| AI289111 | 3932375 |
| AI289120 | 3932384 |
| AI289220 | 3932484 |
| AI289278 | 3932542 |
| AI290658 | 3933432 |
| AI290826 | 3933600 |
| AI291206 | 3933969 |
| AI291247 | 3934021 |
| AI291296 | 3934070 |
| AI291583 | 3934357 |
| AI292299 | 3935073 |
| AI298059 | 3957795 |
| AI298459 | 3958195 |
| AI298496 | 3958232 |
| AI298716 | 3958452 |
| AI298972 | 3958626 |
| AI299524 | 3959109 |
| A1299945 | 3959291 |
| AI300489 | 3959835 |
| AI300532 | 3959878 |
| AI300553 | 3959899 |
| AI300562 | 3959908 |
| AI301532 | 3960878 |
| AI301933 | 3961279 |
| AI302083 | 3961429 |
| AI302102 | 3961448 |
| AI302137 | 3961483 |
| AI302526 | 3961872 |
| AI302799 | 3962145 |
| AI304935 | 3988624 |
| AI304947 | 3988636 |
| AI305530 | 3990421 |
| AI305838 | 3988967 |
| AI306421 | 3989492 |
| AI306457 | 3989528 |
| AI306464 | 3989535 |
| AI307302 | 4002027 |
| AI307402 | 4002127 |
| AI307419 | 4002144 |
| AI307515 | 4002240 |
| AI307634 | 4001847 |
| AI308072 | 4002707 |
| AI308800 | 4003671 |
| AI309025 | 4003896 |
| AI309121 | 4003992 |
| AI309238 | 4004109 |
| AI309401 | 4004272 |
| AI309843 | 4004714 |
| AI310138 | 4005009 |
| AI310235 | 4005106 |
| AI310465 | 4005336 |
| AI310484 | 4005355 |
| AI312049 | 4017654 |
| AI312130 | 4017735 |
| AI312325 | 4017930 |
| AI312542 | 4018147 |
| AI312552 | 4018157 |
| AI312562 | 4018167 |
| AI313352 | 4018957 |
| AI313387 | 4018992 |
| AI318091 | 4033872 |
| AI318280 | 4034139 |
| AI332459 | 4069018 |
| AI332588 | 4069147 |
| AI332597 | 4069156 |
| AI332698 | 4069257 |
| AI332712 | 4069271 |
| AI332904 | 4069463 |
| AI332939 | 4069498 |
| AI333116 | 4069675 |
| AI333548 | 4070107 |
| AI334833 | 4071760 |
| AI334964 | 4071891 |
| AI334996 | 4071923 |
| AI335028 | 4071955 |
| AI335153 | 4072080 |
| AI335178 | 4072105 |
| AI335269 | 4072196 |
| AI335554 | 4072481 |
| AI335829 | 4072756 |
| AI336147 | 4073074 |
| AI336326 | 4073253 |
| AI336501 | 4073428 |
| AI336800 | 4073727 |
| AI336949 | 4073876 |
| AI338190 | 4075117 |
| AI338222 | 4075149 |
| AI338335 | 4075262 |
| AI338338 | 4075265 |
| AI338379 | 4075306 |
| AI338603 | 4075530 |
| AI338817 | 4075744 |
| AI339354 | 4076281 |
| AI339458 | 4076385 |
| AI340044 | 4076971 |
| AI340246 | 4077173 |
| AI340533 | 4077460 |
| AI340582 | 4077509 |
| AI340611 | 4077538 |
| AI340627 | 4077554 |
| AI340981 | 4077908 |
| AI341076 | 4078003 |
| AI341334 | 4078261 |
| AI341352 | 4078279 |
| AI341551 | 4078478 |
| AI341700 | 4078627 |
| AI341972 | 4078899 |
| AI342269 | 4079196 |
| AI342409 | 4079336 |
| AI342468 | 4079395 |
| AI342546 | 4079473 |
| AI342669 | 4079875 |
| AI342937 | 4080143 |
| AI343091 | 4080297 |
| AI343112 | 4080318 |
| AI343367 | 4080573 |
| AI343404 | 4080610 |
| AI343604 | 4080810 |
| AI343692 | 4080898 |
| AI345347 | 4082553 |
| AI345370 | 4082576 |
| AI345416 | 4082622 |
| AI345478 | 4082684 |
| AI346021 | 4083224 |
| AI346381 | 4083587 |
| AI346657 | 4083863 |
| AI346809 | 4084015 |
| AI347155 | 4084361 |
| AI347161 | 4084667 |
| AI347473 | 4084679 |
| AI347506 | 4084712 |
| AI348579 | 4085785 |
| AI349099 | 4086305 |
| AI349256 | 4086462 |
| AI349452 | 4086658 |
| AI349937 | 4087143 |
| AI350388 | 4087594 |
| AI351045 | 4088251 |
| AI351780 | 4088986 |
| AI352032 | 4089238 |
| AI352141 | 4089347 |
| AI355050 | 4095203 |
| AI356309 | 4107930 |
| AI356718 | 4108339 |
| AI356805 | 4108426 |
| AI356967 | 4108577 |
| AI357346 | 4108967 |
| AI357472 | 4109093 |
| AI357642 | 4109263 |
| AI357779 | 4109400 |
| AI358685 | 4110306 |
| AI358723 | 4110344 |
| AI359849 | 4111470 |
| AI360123 | 4111744 |
| AI360165 | 4111786 |
| AI360451 | 4112072 |
| AI360710 | 4112331 |
| AI360870 | 4112491 |
| AI361119 | 4112740 |
| AI361256 | 4112877 |
| AI361377 | 4112998 |
| AI361905 | 4113526 |
| AI361965 | 4113586 |
| AI361969 | 4113590 |
| AI362403 | 4114024 |
| AI362462 | 4114083 |
| AI362490 | 4114111 |
| AI363118 | 4114739 |
| AI364053 | 4123742 |
| AI364592 | 4124281 |
| AI364592 | 4124281 |
| AI365603 | 4125292 |
| AI365603 | 4125292 |
| AI365612 | 4125301 |
| AI365612 | 4125301 |
| AI366365 | 4126054 |
| AI366365 | 4126054 |
| AI366374 | 4126063 |
| AI366376 | 4126065 |
| AI366376 | 4126065 |
| AI366380 | 4126069 |
| AI366380 | 4126069 |
| AI366381 | 4126070 |
| AI366459 | 4126148 |
| AI366459 | 4126148 |
| AI366461 | 4126150 |
| AI366467 | 4126156 |
| AI366467 | 4126156 |
| AI366471 | 4126160 |
| AI366549 | 4126238 |
| AI366549 | 4126238 |
| AI367350 | 4137095 |
| AI367641 | 4137386 |
| AI367730 | 4137475 |
| AI367759 | 4137504 |
| AI368048 | 4137793 |
| AI368114 | 4137859 |
| AI368664 | 4147417 |
| AI368743 | 4147496 |
| AI369174 | 4147927 |
| AI369849 | 4148602 |
| AI370846 | 4149599 |
| AI371832 | 4150585 |
| AI372599 | 4152465 |
| AI373120 | 4152986 |
| AI373289 | 4153155 |
| AI373323 | 4153189 |
| AI373544 | 4153410 |
| AI373565 | 4153431 |
| AI373634 | 4153500 |
| AI374628 | 4174618 |
| AI374643 | 4174633 |
| AI374954 | 4174944 |
| AI374954 | 4174944 |
| AI375141 | 4175131 |
| AI375141 | 4175131 |
| AI375465 | 4175455 |
| AI375465 | 4175455 |
| AI375751 | 4175741 |
| AI375751 | 4175741 |
| AI375919 | 4175909 |
| AI375919 | 4175909 |
| AI376513 | 4186362 |
| AI376808 | 4186661 |
| AI376840 | 4186693 |
| AI376865 | 4186718 |
| AI377012 | 4186865 |
| AI377194 | 4187047 |
| AI378290 | 4188143 |
| AI378613 | 4188466 |
| AI378631 | 4188484 |
| AI378902 | 4188755 |
| AI378932 | 4188785 |
| AI379402 | 4189255 |
| AI379597 | 4189450 |
| AI379679 | 4189532 |
| AI380537 | 4190390 |
| AI380706 | 4190559 |
| AI380812 | 4190665 |
| AI380842 | 4190695 |
| AI380932 | 4190785 |
| AI381351 | 4194132 |
| AI381649 | 4194430 |
| AI381996 | 4194777 |
| AI382020 | 4194801 |
| AI382065 | 4194846 |
| AI382189 | 4194959 |
| AI382980 | 4195761 |
| AI383637 | 4196418 |
| AI383945 | 4196726 |
| AI391501 | 4217505 |
| AI391631 | 4217635 |
| AI391717 | 4217721 |
| AI392607 | 4222154 |
| AI394173 | 4223720 |
| AI394364 | 4223911 |
| AI394420 | 4223967 |
| AI394469 | 4224016 |
| AI394498 | 4224045 |
| AI394646 | 4224193 |
| AI399717 | 4242804 |
| AI400282 | 4243369 |
| AI400410 | 4243497 |
| AI400511 | 4243598 |
| AI400752 | 4243839 |
| AI401150 | 4244237 |
| AI401220 | 4244307 |
| AI401513 | 4244600 |
| AI401771 | 4244858 |
| AI401840 | 4244927 |
| AI417016 | 4260520 |
| AI417131 | 4260635 |
| AI417562 | 4261066 |
| AI417833 | 4261337 |
| AI417910 | 4261414 |
| AI420026 | 4265957 |
| AI420172 | 4266103 |
| AI420227 | 4266158 |
| AI420373 | 4266304 |
| AI420898 | 4266829 |
| AI421027 | 4266958 |
| AI421249 | 4267180 |
| AI421290 | 4267221 |
| AI421401 | 4267332 |
| AI421412 | 4267343 |
| AI421741 | 4267672 |
| AI421761 | 4267692 |
| AI422243 | 4268174 |
| AI422907 | 4268838 |
| AI423180 | 4269111 |
| AI423541 | 4269472 |
| AI431327 | 4302410 |
| AI431350 | 4302571 |
| AI431675 | 4304843 |
| AI431801 | 4305724 |
| AI431814 | 4305815 |
| AI431869 | 4306201 |
| AI431923 | 4306579 |
| AI432306 | 4309261 |
| AI432462 | 4281890 |
| AI432603 | 4283019 |
| AI432969 | 4285894 |
| AI433717 | 4291133 |
| AI433757 | 4291414 |
| AI433818 | 4291841 |
| AI433976 | 4292947 |
| AI434025 | 4293290 |
| AI434109 | 4293878 |
| AI434150 | 4294165 |
| AI434180 | 4294375 |
| AI434250 | 4294864 |
| AI434639 | 4297586 |
| AI434949 | 4299755 |
| AI435153 | 4301186 |
| AI435349 | 4302561 |
| AI435606 | 4304362 |
| AI435945 | 4306732 |
| AI436714 | 4283904 |
| AI439173 | 4302469 |
| AI439248 | 4302993 |
| AI439452 | 4304421 |
| AI439762 | 4306588 |
| AI439774 | 4306672 |
| AI439987 | 4308001 |
| AI440088 | 4308710 |
| AI440194 | 4309452 |
| AI440238 | 4281227 |
| AI445471 | 4288444 |
| AI445592 | 4289290 |
| AI446003 | 4292165 |
| AI446249 | 4293887 |
| AI446426 | 4295127 |
| AI452485 | 4307457 |
| AI452703 | 4287210 |
| AI452707 | 4287238 |
| AI453181 | 4308561 |
| AI453358 | 4281271 |
| AI453402 | 4281623 |
| AI453477 | 4282222 |
| AI457107 | 4309976 |
| AI457157 | 4310026 |
| AI457216 | 4310085 |
| AI457410 | 4310279 |
| AI457964 | 4311982 |
| AI458457 | 4311036 |
| AI458521 | 4311100 |
| AI458673 | 4311252 |
| AI458743 | 4311322 |
| AI458799 | 4311378 |
| AI458823 | 4311402 |
| AI459234 | 4311813 |
| AI459667 | 4312548 |
| AI460230 | 4313111 |
| AI467858 | 4329948 |
| AI467861 | 4329951 |
| AI468347 | 4330437 |
| AI469038 | 4331128 |
| AI469767 | 4331857 |
| AI470061 | 4332151 |
| AI470259 | 4332349 |
| AI471561 | 4333651 |
| AI472067 | 4334157 |
| AI472525 | 4325570 |
| AI472566 | 4325611 |
| AI473310 | 4326355 |
| AI473779 | 4326824 |
| AI474125 | 4327170 |
| AI474235 | 4327280 |
| AI474466 | 4327511 |
| AI474750 | 4327795 |
| AI475476 | 4328521 |
| AI475810 | 4328855 |
| AI476075 | 4329120 |
| AI476254 | 4329299 |
| AI476310 | 4329355 |
| AI476318 | 4329363 |
| AI478365 | 4371591 |
| AI479009 | 4372177 |
| AI479021 | 4372189 |
| AI479439 | 4372607 |
| AI479948 | 4373116 |
| AI480214 | 4373382 |
| AI480345 | 4373513 |
| AI480424 | 4373592 |
| AI491775 | 4392778 |
| AI492263 | 4393266 |
| AI492953 | 4393956 |
| AI493851 | 4394854 |
| AI493961 | 4394964 |
| AI494299 | 4395302 |
| AI497636 | 4389618 |
| AI497759 | 4389741 |
| AI499331 | 4391313 |
| AI499378 | 4391360 |
| AI499393 | 4391375 |
| AI499439 | 4391421 |
| AI499516 | 4391498 |
| AI499584 | 4391566 |
| AI499933 | 4391915 |
| AI499986 | 4391968 |
| AI500531 | 4392513 |
| AI521100 | 4435235 |
| AI521156 | 4435291 |
| AI521270 | 4435405 |
| AI521964 | 4436099 |
| AI521984 | 4436119 |
| AI523222 | 4437357 |
| AI523506 | 4437641 |
| AI523940 | 4438075 |
| AI523964 | 4438099 |
| AI524304 | 4438439 |
| AI524414 | 4438549 |
| AI524826 | 4438961 |
| AI524874 | 4439009 |
| AI524991 | 4439126 |
| AI525102 | 4439237 |
| AI525231 | 4439366 |
| AI525426 | 4439561 |
| AI525454 | 4439589 |
| AI525552 | 4439687 |
| AI525651 | 4439786 |
| AI525654 | 4439789 |
| AI525702 | 4439837 |
| AI525773 | 4439908 |
| AI525843 | 4439978 |
| AI536024 | 4450159 |
| AI536588 | 4450723 |
| AI536685 | 4450820 |
| AI536833 | 4450968 |
| AI536991 | 4451126 |
| AI537244 | 4451379 |
| AI537498 | 4451633 |
| AI538342 | 4452477 |
| AI538681 | 4452816 |
| AI539349 | 4453484 |
| AI546840 | 4464328 |
| AI547041 | 4464529 |
| AI547068 | 4464556 |
| AI547090 | 4464578 |
| AI547125 | 4464613 |
| AI547146 | 4464634 |
| AI547284 | 4464772 |
| AI547285 | 4464773 |
| AI547309 | 4464797 |
| AI547327 | 4464815 |
| AI553845 | 4486208 |
| AI554016 | 4486379 |
| AI554590 | 4486953 |
| AI554809 | 4487172 |
| AI557053 | 4489416 |
| AI557059 | 4489422 |
| AI557082 | 4489445 |
| AI557112 | 4489475 |
| AI557116 | 4489479 |
| AI557118 | 4489481 |
| AI557182 | 4489545 |
| AI557225 | 4489588 |
| AI557226 | 4489589 |
| AI557231 | 4489594 |
| AI557235 | 4489598 |
| AI557237 | 4489600 |
| AI557243 | 4489606 |
| AI557246 | 4489609 |
| AI557269 | 4489632 |
| AI557277 | 4489640 |
| AI557336 | 4489699 |
| AI557338 | 4489701 |
| AI557363 | 4489726 |
| AI557452 | 4489815 |
| AI557458 | 4489821 |
| AI557495 | 4489858 |
| AI557599 | 4489962 |
| AI557626 | 4489989 |
| AI557639 | 4490002 |
| AI559299 | 4509504 |
| AI559680 | 4509885 |
| AI559903 | 4510108 |
| AI559999 | 4510204 |
| AI560184 | 4510525 |
| AI560756 | 4511097 |
| AI560870 | 4511211 |
| AI560958 | 4511299 |
| AI561122 | 4511463 |
| AI563921 | 4522378 |
| AI564173 | 4522630 |
| AI564269 | 4522726 |
| AI564475 | 4522932 |
| AI564487 | 4522944 |
| AI564506 | 4522963 |
| AI564822 | 4523279 |
| AI565591 | 4524048 |
| AI566084 | 4524536 |
| AI566490 | 4524942 |
| AI567102 | 4525554 |
| AI569556 | 4532930 |
| AI569819 | 4533193 |
| AI569955 | 4533329 |
| AI570128 | 4533502 |
| AI570141 | 4533515 |
| AI570221 | 4533595 |
| AI570349 | 4533723 |
| AI570521 | 4533895 |
| AI570611 | 4533985 |
| AI570859 | 4534233 |
| AI570942 | 4534316 |
| AI571069 | 4534443 |
| AI571418 | 4534792 |
| AI571517 | 4534891 |
| AI571730 | 4535104 |
| AI572096 | 4535470 |
| AI573140 | 4536514 |
| AI573212 | 4536586 |
| AI573218 | 4536592 |
| AI580160 | 4564536 |
| AI580162 | 4564538 |
| AI580176 | 4564552 |
| AI580927 | 4565303 |
| AI581840 | 4567737 |
| AI582365 | 4568262 |
| AI582912 | 4568809 |
| AI583257 | 4569154 |
| AI587118 | 4573559 |
| AI587300 | 4573741 |
| AI587642 | 4574083 |
| AI588087 | 4597134 |
| AI588866 | 4597914 |
| AI589075 | 4598123 |
| AI589206 | 4598254 |
| AI589285 | 4598333 |
| AI589476 | 4598524 |
| AI589533 | 4598581 |
| AI589633 | 4598681 |
| AI589756 | 4598804 |
| AI590195 | 4599243 |
| AI590351 | 4599399 |
| AI590878 | 4599926 |
| AI591088 | 4600136 |
| AI597980 | 4607028 |
| AI598180 | 4607228 |
| AI608684 | 4617851 |
| AI608968 | 4618135 |
| AI609659 | 4618826 |
| AI610412 | 4619579 |
| AI610699 | 4619866 |
| AI610920 | 4620087 |
| AI611738 | 4620905 |
| AI612873 | 4622040 |
| AI613093 | 4622260 |
| AI619498 | 4628624 |
| AI619798 | 4628924 |
| AI620111 | 4629237 |
| AI620284 | 4629410 |
| AI620427 | 4629553 |
| AI620673 | 4629799 |
| AI620796 | 4629922 |
| AI620924 | 4630050 |
| AI621153 | 4630279 |
| AI623176 | 4648101 |
| AI623315 | 4648240 |
| AI623804 | 4648735 |
| AI623891 | 4648822 |
| AI623924 | 4648855 |
| AI623984 | 4648915 |
| AI624249 | 4649180 |
| AI624441 | 4649372 |
| AI624709 | 4649640 |
| AI625201 | 4650132 |
| AI625204 | 4650135 |
| AI625404 | 4650335 |
| AI625747 | 4650678 |
| AI625864 | 4650795 |
| AI627356 | 4664156 |
| AI627542 | 4664342 |
| AI627553 | 4664353 |
| AI627750 | 4664550 |
| AI627818 | 4664618 |
| AI627945 | 4664745 |
| AI628389 | 4665189 |
| AI628609 | 4665409 |
| AI628885 | 4665685 |
| AI630104 | 4681434 |
| AI630118 | 4681448 |
| AI630144 | 4681474 |
| AI630188 | 4681518 |
| AI630228 | 4681558 |
| AI630282 | 4681612 |
| AI630306 | 4681636 |
| AI630345 | 4681675 |
| AI630357 | 4681687 |
| AI630362 | 4681692 |
| AI630584 | 4681914 |
| AI630616 | 4681946 |
| AI630634 | 4681964 |
| AI630685 | 4682015 |
| AI630891 | 4682221 |
| AI630985 | 4682315 |
| AI631703 | 4683033 |
| AI631745 | 4683075 |
| AI632108 | 4683438 |
| AI632244 | 4683574 |
| AI632523 | 4683853 |
| AI632534 | 4683864 |
| AI633646 | 4684976 |
| AI634228 | 4685558 |
| AI634702 | 4686032 |
| AI634740 | 4686070 |
| AI635096 | 4686426 |
| AI635209 | 4686539 |
| AI635275 | 4686605 |
| AI635310 | 4686640 |
| AI635478 | 4686808 |
| AI635658 | 4686988 |
| AI635667 | 4686997 |
| AI635681 | 4687011 |
| AI636577 | 4687907 |
| AI636626 | 4687956 |
| AI636628 | 4687958 |
| AI636701 | 4688031 |
| AI636899 | 4688229 |
| AI636911 | 4688241 |
| AI636943 | 4688273 |
| AI637584 | 4689818 |
| AI638327 | 4690561 |
| AI638414 | 4690648 |
| AI638492 | 4690726 |
| AI640588 | 4703697 |
| AI648682 | 4729516 |
| AI648684 | 4729518 |
| AI650721 | 4734700 |
| AI651069 | 4735048 |
| AI651195 | 4735174 |
| AI651974 | 4735953 |
| AI651976 | 4735955 |
| AI652084 | 4736063 |
| AI652112 | 4736091 |
| AI652555 | 4736534 |
| AI653319 | 4737298 |
| AI653367 | 4737346 |
| AI653541 | 4737520 |
| AI653621 | 4737600 |
| AI654122 | 4738101 |
| AI654123 | 4738102 |
| AI654183 | 4738162 |
| AI654284 | 4738263 |
| AI654454 | 4738433 |
| AI655189 | 4739168 |
| AI656122 | 4740101 |
| AI656158 | 4740137 |
| AI656323 | 4740302 |
| AI656402 | 4740381 |
| AI658689 | 4762259 |
| AI658850 | 4762420 |
| AI659354 | 4762924 |
| AI659386 | 4762956 |
| AI659440 | 4763010 |
| AI659898 | 4763468 |
| AI660243 | 4763813 |
| AI660356 | 4763926 |
| AI660606 | 4764189 |
| AI660919 | 4764502 |
| AI669052 | 4833826 |
| AI669210 | 4833984 |
| AI669253 | 4834027 |
| AI669704 | 4834478 |
| AI670002 | 4834776 |
| AI670009 | 4834783 |
| AI670913 | 4850644 |
| AI671180 | 4850911 |
| AI671394 | 4851125 |
| AI671555 | 4851286 |
| AI671642 | 4851373 |
| AI672201 | 4851932 |
| AI672362 | 4852093 |
| AI672868 | 4852599 |
| AI674583 | 4875063 |
| AI674586 | 4875066 |
| AI674594 | 4875074 |
| AI674871 | 4875351 |
| AI675889 | 4876369 |
| AI677810 | 4887992 |
| AI678281 | 4888463 |
| AI678832 | 4889014 |
| AI679619 | 4889801 |
| AI679629 | 4889811 |
| AI680218 | 4890400 |
| AI680242 | 4890424 |
| AI680343 | 4890525 |
| AI680416 | 4890598 |
| AI680506 | 4890688 |
| AI680874 | 4891056 |
| AI681405 | 4891587 |
| AI682088 | 4892270 |
| AI682287 | 4892469 |
| AI682362 | 4892544 |
| AI682937 | 4893119 |
| AI683094 | 4893286 |
| AI683183 | 4893365 |
| AI683298 | 4893480 |
| AI683431 | 4893613 |
| AI684039 | 4895333 |
| AI684157 | 4895451 |
| AI684170 | 4895464 |
| AI685124 | 4896418 |
| AI685632 | 4896926 |
| AI686139 | 4897433 |
| AI686325 | 4897619 |
| AI686779 | 4898073 |
| AI686957 | 4898251 |
| AI687362 | 4898656 |
| AI688098 | 4899392 |
| AI688187 | 4899481 |
| AI688573 | 4899867 |
| AI688807 | 4900101 |
| AI689722 | 4901016 |
| AI689762 | 4901056 |
| AI690157 | 4901451 |
| AI690236 | 4901530 |
| AI690278 | 4901572 |
| AI690979 | 4902281 |
| AI690986 | 4902288 |
| AI691072 | 4902374 |
| AI692513 | 4969853 |
| AI692940 | 4970280 |
| AI692995 | 4970335 |
| AI693198 | 4970538 |
| AI693302 | 4970642 |
| AI693493 | 4970833 |
| AI693561 | 4970901 |
| AI693674 | 4971014 |
| AI693697 | 4971037 |
| AI694087 | 4971427 |
| AI694199 | 4971539 |
| AI694211 | 4971551 |
| AI694320 | 4971660 |
| AI694477 | 4971817 |
| AI694572 | 4971912 |
| AI694596 | 4971936 |
| AI694767 | 4982667 |
| AI696488 | 4984388 |
| AI696513 | 4984413 |
| AI696693 | 4984593 |
| AI696992 | 4984892 |
| AI697249 | 4985149 |
| AI697470 | 4985370 |
| AI697599 | 4985499 |
| AI697815 | 4985715 |
| AI697849 | 4985749 |
| AI697873 | 4985773 |
| AI698092 | 4985992 |
| AI698198 | 4986098 |
| AI700228 | 4988128 |
| AI700642 | 4988542 |
| AI701119 | 4989019 |
| AI701140 | 4989040 |
| AI703256 | 4991156 |
| AI703405 | 4991305 |
| AI708172 | 4997948 |
| AI708364 | 4998140 |
| AI708884 | 4998660 |
| AI708995 | 4998771 |
| AI709186 | 4998962 |
| AI709356 | 4999132 |
| AI718780 | 5036036 |
| AI718825 | 5036081 |
| AI719425 | 5036681 |
| AI719701 | 5036957 |
| AI720132 | 5037426 |
| AI720354 | 5037610 |
| AI720403 | 5037659 |
| AI720603 | 5037859 |
| AI732097 | 5053232 |
| AI732248 | 5053361 |
| AI732541 | 5053654 |
| AI732557 | 5053670 |
| AI732680 | 5053793 |
| AI732784 | 5053897 |
| AI733116 | 5054229 |
| AI733857 | 5054970 |
| AI733862 | 5054975 |
| AI734209 | 5055322 |
| AI734266 | 5055379 |
| AI734854 | 5056378 |
| AI735314 | 5056838 |
| AI735683 | 5057207 |
| AI738488 | 5100469 |
| AI738605 | 5100586 |
| AI739102 | 5101083 |
| AI740534 | 5108822 |
| AI740792 | 5109080 |
| AI740796 | 5109084 |
| AI740869 | 5109157 |
| AI740900 | 5109188 |
| AI741143 | 5109431 |
| AI741192 | 5109480 |
| AI741213 | 5109501 |
| AI741354 | 5109642 |
| AI741446 | 5109734 |
| AI741486 | 5109774 |
| AI741633 | 5109921 |
| AI741843 | 5110131 |
| AI741861 | 5110236 |
| AI742115 | 5110403 |
| AI742468 | 5110756 |
| AI742912 | 5111200 |
| AI743303 | 5111591 |
| AI743852 | 5112140 |
| AI743954 | 5112242 |
| AI744451 | 5112739 |
| AI744509 | 5112797 |
| AI744810 | 5113098 |
| AI744818 | 5113106 |
| AI745218 | 5113506 |
| AI745280 | 5113568 |
| AI749547 | 5127811 |
| AI749886 | 5128150 |
| AI750535 | 5128799 |
| AI750567 | 5128831 |
| AI750682 | 5128946 |
| AI750846 | 5129110 |
| AI751364 | 5129628 |
| AI751565 | 5129829 |
| AI751583 | 5129847 |
| AI751914 | 5130178 |
| AI751930 | 5130194 |
| AI752546 | 5130810 |
| AI752822 | 5131086 |
| AI752913 | 5131177 |
| AI753229 | 5131493 |
| AI753269 | 5131533 |
| AI753280 | 5131544 |
| AI753459 | 5131723 |
| AI753662 | 5131926 |
| AI753664 | 5131928 |
| AI753784 | 5132136 |
| AI753951 | 5132215 |
| AI754013 | 5132277 |
| AI754071 | 5132335 |
| AI754437 | 5132701 |
| AI754461 | 5132725 |
| AI754741 | 5133005 |
| AI754874 | 5133138 |
| AI755085 | 5133349 |
| AI755142 | 5133406 |
| AI758378 | 5152101 |
| AI760699 | 5176366 |
| AI760827 | 5176494 |
| AI761098 | 5176765 |
| AI761110 | 5176777 |
| AI761190 | 5176857 |
| AI761291 | 5176958 |
| AI761469 | 5177136 |
| AI761508 | 5177175 |
| AI761702 | 5177458 |
| AI761728 | 5177484 |
| AI761986 | 5177653 |
| AI762085 | 5177752 |
| AI762634 | 5178301 |
| AI763239 | 5178906 |
| AI764962 | 5231471 |
| AI766083 | 5232592 |
| AI766123 | 5232632 |
| AI766514 | 5233023 |
| AI767050 | 5233559 |
| A1767430 | 5233859 |
| AI767692 | 5234201 |
| AI768132 | 5234641 |
| AI768465 | 5234974 |
| AI768568 | 5235077 |
| AI768624 | 5235133 |
| AI768848 | 5235357 |
| AI768854 | 5235363 |
| AI768877 | 5235386 |
| AI768908 | 5235417 |
| AI769145 | 5235654 |
| AI769392 | 5235901 |
| AI769478 | 5235987 |
| AI769975 | 5236484 |
| AI770054 | 5236650 |
| AI770098 | 5236553 |
| AI791127 | 5338843 |
| AI791165 | 5338881 |
| AI791173 | 5338889 |
| AI791179 | 5338895 |
| AI791370 | 5339086 |
| AI791419 | 5339135 |
| AI791548 | 5339264 |
| AI791719 | 5339519 |
| AI791906 | 5339622 |
| AI791998 | 5339714 |
| AI792191 | 5339907 |
| AI792758 | 5340474 |
| AI793062 | 5340778 |
| AI793283 | 5340999 |
| AI796120 | 5361583 |
| AI796507 | 5361970 |
| AI796560 | 5362023 |
| AI796562 | 5362025 |
| AI796571 | 5362034 |
| AI797042 | 5362514 |
| AI797353 | 5362825 |
| AI798840 | 5364312 |
| AI799502 | 5364974 |
| AI799699 | 5365171 |
| AI799891 | 5365363 |
| AI799974 | 5365446 |
| AI800048 | 5365520 |
| AI800250 | 5365722 |
| AI800673 | 5366067 |
| AI800896 | 5366368 |
| AI801373 | 5366845 |
| AI801589 | 5367061 |
| AI801810 | 5367282 |
| AI802154 | 5367626 |
| AI802542 | 5368014 |
| AI802946 | 5368407 |
| AI802998 | 5368470 |
| AI804327 | 5369799 |
| AI804346 | 5369818 |
| AI804662 | 5370134 |
| AI804733 | 5370205 |
| AI804983 | 5391573 |
| AI805364 | 5391930 |
| AI805379 | 5391945 |
| AI805522 | 5392088 |
| AI806044 | 5392610 |
| AI806161 | 5392727 |
| AI806996 | 5393562 |
| AI808505 | 5395071 |
| AI808684 | 5395250 |
| AI809314 | 5395880 |
| AI809963 | 5396529 |
| AI830002 | 5396568 |
| AI810626 | 5397192 |
| AI810698 | 5397264 |
| AI811144 | 5397710 |
| AI811498 | 5398064 |
| AI813724 | 5424939 |
| AI813758 | 5424973 |
| AI813772 | 5424987 |
| AI813937 | 5425152 |
| AI814148 | 5425363 |
| AI815033 | 5426248 |
| AI815196 | 5426401 |
| AI815380 | 5430926 |
| AI815416 | 5430962 |
| AI815449 | 5430995 |
| AI815454 | 5431000 |
| AI815495 | 5431041 |
| AI815497 | 5431043 |
| AI815498 | 5431044 |
| AI815499 | 5431045 |
| AI815664 | 5431210 |
| AI815677 | 5431223 |
| AI815703 | 5431249 |
| AI815829 | 5431375 |
| AI815966 | 5431512 |
| AI815972 | 5431518 |
| AI815976 | 5431522 |
| AI815987 | 5431533 |
| AI816033 | 5431579 |
| AI816058 | 5431604 |
| AI816102 | 5431648 |
| AI816235 | 5431781 |
| AI816313 | 5431859 |
| AI816322 | 5431868 |
| AI816938 | 5436017 |
| AI817242 | 5436310 |
| AI817249 | 5436317 |
| AI817432 | 5436511 |
| AI818255 | 5437334 |
| AI819014 | 5438178 |
| AI819225 | 5438304 |
| AI819564 | 5438643 |
| AI819701 | 5438780 |
| AI820995 | 5440074 |
| AI821437 | 5440516 |
| AI823661 | 5444332 |
| AI825408 | 5446079 |
| AI825504 | 5446175 |
| AI825799 | 5446470 |
| AI825988 | 5446659 |
| AI826105 | 5446776 |
| AI826427 | 5447098 |
| AI826504 | 5447175 |
| AI827129 | 5447800 |
| AI827550 | 5448221 |
| AI827667 | 5448338 |
| AI828602 | 5449273 |
| AI828670 | 5449341 |
| AI829303 | 5449974 |
| AI829784 | 5450455 |
| AI830758 | 5451514 |
| AI831047 | 5451718 |
| AI831462 | 5452133 |
| AI831502 | 5452173 |
| AI832609 | 5454589 |
| AI858066 | 5511682 |
| AI859156 | 5512772 |
| AI859442 | 5513058 |
| AI859619 | 5513235 |
| AI860016 | 5513632 |
| AI860714 | 5514330 |
| AI861878 | 5525985 |
| AI861997 | 5526104 |
| AI863042 | 5527149 |
| AI864040 | 5528147 |
| AI864870 | 5528977 |
| AI864927 | 5529034 |
| AI865716 | 5529823 |
| AI866262 | 5530369 |
| AI866551 | 5530658 |
| AI867463 | 5540479 |
| AI867729 | 5540745 |
| AI868421 | 5541437 |
| AI869280 | 5543248 |
| AI869322 | 5543290 |
| AI871027 | 5544995 |
| AI871477 | 5545526 |
| AI871745 | 5545717 |
| AI872085 | 5546134 |
| AI873501 | 5547550 |
| AI878846 | 5552895 |
| AI878878 | 5552927 |
| AI878918 | 5552967 |
| AI878926 | 5552975 |
| AI879010 | 5553059 |
| AI879036 | 5553085 |
| AI879040 | 5553089 |
| AI879059 | 5553108 |
| AI879179 | 5553228 |
| AI879242 | 5553291 |
| AI879248 | 5553297 |
| AI879992 | 5554041 |
| AI879995 | 5554044 |
| AI880872 | 5554921 |
| AI884578 | 5589742 |
| AI884829 | 5589993 |
| AI884963 | 5590127 |
| AI885174 | 5590338 |
| AI885833 | 5590997 |
| AI886418 | 5591582 |
| AI887517 | 5592681 |
| AI887632 | 5592796 |
| AI887664 | 5592828 |
| AI887875 | 5593039 |
| AI888438 | 5593525 |
| AI888493 | 5593657 |
| AI889347 | 5594511 |
| AI889759 | 5594923 |
| AI890240 | 5595404 |
| AI890387 | 5595551 |
| AI890616 | 5595780 |
| AI910526 | 5630262 |
| AI910552 | 5630288 |
| AI911059 | 5630795 |
| AI911703 | 5631558 |
| AI911776 | 5631631 |
| AI912057 | 5631912 |
| AI912414 | 5632269 |
| AI913323 | 5633097 |
| AI914030 | 5633885 |
| AI914128 | 5633983 |
| AI914133 | 5633988 |
| AI914231 | 5634086 |
| AI915264 | 5635119 |
| AI916374 | 5636151 |
| AI916419 | 5636196 |
| AI916584 | 5636439 |
| AI917110 | 5636965 |
| AI917649 | 5637504 |
| AI921254 | 5657218 |
| AI921434 | 5657398 |
| AI922040 | 5658004 |
| AI922114 | 5658078 |
| AI922760 | 5658724 |
| AI922855 | 5658819 |
| AI923011 | 5658901 |
| AI923485 | 5659449 |
| AI923574 | 5659538 |
| AI923627 | 5659591 |
| AI923978 | 5659942 |
| AI924598 | 5660562 |
| AI924963 | 5660927 |
| AI925735 | 5661699 |
| AI927301 | 5663265 |
| AI927636 | 5663600 |
| AI927846 | 5663810 |
| AI928622 | 5664586 |
| AI928889 | 5664853 |
| AI929113 | 5665077 |
| AI929339 | 5665303 |
| AI929453 | 5665417 |
| AI929464 | 5665428 |
| AI929509 | 5665473 |
| AI929611 | 5665575 |
| AI929664 | 5665628 |
| AI932951 | 5671688 |
| AI933533 | 5672270 |
| AI934153 | 5673023 |
| AI934595 | 5673392 |
| AI935291 | 5674161 |
| AI935704 | 5674574 |
| AI935710 | 5674580 |
| AI935816 | 5674686 |
| AI936472 | 5675342 |
| AI936548 | 5675418 |
| AI936562 | 5675432 |
| AI937060 | 5675930 |
| AI937150 | 5676020 |
| AI937296 | 5676166 |
| AI940170 | 5687151 |
| AI948503 | 5740813 |
| AI948513 | 5740823 |
| AI948822 | 5741132 |
| AI949242 | 5741552 |
| AI949422 | 5741820 |
| AI951021 | 5743331 |
| AI951049 | 5743359 |
| AI951118 | 5743428 |
| AI951536 | 5743846 |
| AI951970 | 5744280 |
| AI952286 | 5744596 |
| AI952294 | 5744604 |
| AI952763 | 5745073 |
| AI953152 | 5745548 |
| AI953711 | 5746021 |
| AI954167 | 5746477 |
| AI954406 | 5746716 |
| AI954458 | 5746768 |
| AI954585 | 5746895 |
| AI954800 | 5747110 |
| AI955087 | 5747409 |
| AI955769 | 5746079 |
| AI961028 | 5753809 |
| AI961413 | 5754126 |
| AI961592 | 5754305 |
| AI961840 | 5754553 |
| AI962319 | 5755032 |
| AI962736 | 5755449 |
| AI963623 | 5756401 |
| AI968069 | 5764887 |
| AI968159 | 5764977 |
| AI968311 | 5765129 |
| AI968401 | 5765219 |
| AI969567 | 5766385 |
| AI969804 | 5766622 |
| AI970381 | 5767207 |
| AI970594 | 5767420 |
| AI970919 | 5767745 |
| AI971281 | 5168107 |
| AI971655 | 5768481 |
| AI972279 | 5769105 |
| AI972479 | 5769395 |
| AI973022 | 5769848 |
| AI973109 | 5769935 |
| AI973126 | 5769952 |
| AI973218 | 5770044 |
| AI978832 | 5803862 |
| AI979288 | 5804307 |
| AI983220 | 5810439 |
| AI983430 | 5810649 |
| AI984541 | 5811818 |
| AI984636 | 5811913 |
| AI984763 | 5812040 |
| AI985122 | 5812399 |
| AI986240 | 5813517 |
| AI989530 | 5836411 |
| AI989600 | 5836481 |
| AI991281 | 5838186 |
| AI991469 | 5838374 |
| AI991546 | 5838451 |
| AJ000041 | 2661372 |
| AJ000644 | 2695707 |
| AJ000881 | 2924308 |
| AJ001189 | 3059134 |
| AJ001306 | 2370148 |
| AJ001381 | 2764616 |
| AJ001443 | 6006514 |
| AJ001838 | 2832730 |
| AJ002744 | 6318185 |
| AJ002955 | 2632122 |
| AJ004832 | 2982500 |
| AJ004913 | 3288462 |
| AJ004955 | 3005935 |
| AJ005016 | 3005930 |
| AJ005259 | 3043444 |
| AJ005821 | 3123571 |
| AJ005893 | 3114819 |
| AJ006470 | 3687321 |
| AJ006834 | 3236104 |
| AJ007398 | 3668140 |
| AJ007714 | 4938303 |
| AJ010046 | 4127946 |
| AJ010069 | 3483012 |
| AJ010071 | 3483016 |
| AJ010442 | 3954884 |
| AJ010444 | 3954888 |
| AJ010482 | 5689735 |
| AJ010842 | 3646129 |
| AJ010953 | 3646133 |
| AJ011001 | 4456466 |
| AJ011007 | 4468340 |
| AJ011497 | 4128014 |
| AJ012463 | 3850049 |
| AJ012499 | 5441359 |
| AJ130733 | 4995298 |
| AJ131753 | 6273456 |
| AJ132583 | 4210725 |
| AJ132637 | 5689741 |
| AJ132694 | 4454210 |
| AJ133005 | 4585654 |
| AJ223075 | 3355596 |
| AJ223183 | 3925598 |
| AJ223350 | 3255986 |
| AJ223352 | 3255996 |
| AJ223353 | 3255998 |
| AJ223812 | 2894518 |
| AJ224172 | 4107230 |
| AJ224326 | 2894531 |
| AJ224819 | 3133091 |
| AJ224875 | 2996577 |
| AJ227918 | 3183971 |
| AJ228139 | 4585698 |
| AJ237946 | 5701849 |
| AJ238095 | 5262855 |
| AJ242829 | 4995304 |
| AJ243229 | 5706019 |
| AJ243310 | 5262358 |
| AJ243874 | 5911829 |
| AJ249248 | 5834594 |
| AJ249731 | 5921469 |
| AJ250042 | 6013005 |
| AL021683 | 3217033 |
| AL022097 | 3169107 |
| AL031003 | 4007185 |
| AL031346 | 4760290 |
| AL031777 | 10198609 |
| AL034399 | 4493484 |
| AL034555 | 4455444 |
| AL035304 | 4200231 |
| AL035666 | 4914532 |
| AL035731 | 5405368 |
| AL035756 | 5927575 |
| AL035886 | 5927605 |
| AL035985 | 5405615 |
| AL035998 | 5405628 |
| AL036026 | 5405652 |
| AL036123 | 5405743 |
| AL036165 | 5927669 |
| AL036169 | 5405781 |
| AL036170 | 5927671 |
| AL036354 | 5405935 |
| AL036415 | 5405971 |
| AL036423 | 5405978 |
| AL036445 | 5405997 |
| AL036483 | 5927775 |
| AL036499 | 5406046 |
| AL036575 | 5406117 |
| AL036594 | 5406134 |
| AL036650 | 5406181 |
| AL036763 | 5927902 |
| AL036764 | 5927903 |
| AL036801 | 5927917 |
| AL036890 | 5406374 |
| AL036902 | 5927947 |
| AL036988 | 5927998 |
| AL037031 | 5928012 |
| AL037053 | 5928023 |
| AL037105 | 5406560 |
| AL037226 | 5406655 |
| AL037256 | 5866490 |
| AL037267 | 5866492 |
| AL037316 | 5406727 |
| AL037456 | 5928118 |
| AL037471 | 5928129 |
| AL037583 | 5928197 |
| AL037638 | 5406999 |
| AL037646 | 5928237 |
| AL037707 | 5407056 |
| AL037724 | 5407071 |
| AL037732 | 5407078 |
| AL037798 | 5407134 |
| AL037800 | 5407136 |
| AL037812 | 5407147 |
| AL037828 | 5407161 |
| AL037847 | 5407177 |
| AL037895 | 5407219 |
| AL038058 | 5407366 |
| AL038162 | 5407455 |
| AL038234 | 5407512 |
| AL038242 | 5407519 |
| AL038395 | 5407632 |
| AL038549 | 5928464 |
| AL038660 | 5407826 |
| AL038721 | 5407871 |
| AL038802 | 5407936 |
| AL038821 | 5407952 |
| AL038985 | 5408101 |
| AL039175 | 5408254 |
| AL039208 | 5408284 |
| AL039235 | 5408309 |
| AL039253 | 5408327 |
| AL039254 | 5408328 |
| AL039293 | 5408365 |
| AL039349 | 5408416 |
| AL039399 | 5928553 |
| AL039430 | 5928565 |
| AL039507 | 5408549 |
| AL039517 | 5408559 |
| AL039542 | 5408583 |
| AL039599 | 5408636 |
| AL039691 | 5408719 |
| AL039698 | 5408726 |
| AL039814 | 5408819 |
| AL040084 | 5409054 |
| AL040204 | 5409168 |
| AL040264 | 5409223 |
| AL040354 | 5409308 |
| AL040505 | 5409454 |
| AL040518 | 5409467 |
| AL040632 | 5409579 |
| AL040676 | 5409622 |
| AL040692 | 5409638 |
| AL040873 | 5409818 |
| AL040936 | 5409880 |
| AL040993 | 5409936 |
| AL041012 | 5409955 |
| AL041050 | 5409992 |
| AL041176 | 5410111 |
| AL041265 | 5410192 |
| AL041470 | 5420821 |
| AL041780 | 5421127 |
| AL042234 | 5421578 |
| AL042316 | 5421658 |
| AL042404 | 5421751 |
| AL042536 | 5935481 |
| AL042613 | 5935505 |
| AL042815 | 5422257 |
| AL042896 | 5422331 |
| AL043034 | 5422457 |
| AL043046 | 5422467 |
| AL043048 | 5935692 |
| AL043071 | 5422490 |
| AL043113 | 5935737 |
| AL043205 | 5935801 |
| AL043232 | 5935821 |
| AL043245 | 5935830 |
| AL043257 | 5935838 |
| AL043346 | 5422736 |
| AL043506 | 5422893 |
| AL043702 | 5423088 |
| AL043864 | 5935908 |
| AL044016 | 5432244 |
| AL044040 | 5432268 |
| AL044350 | 5432572 |
| AL044447 | 5432665 |
| AL044649 | 5432864 |
| AL044723 | 5432935 |
| AL044891 | 5433089 |
| AL044985 | 5433170 |
| AL045040 | 5433219 |
| AL045213 | 5433375 |
| AL045297 | 5433457 |
| AL045331 | 5433489 |
| AL045332 | 5935985 |
| AL045447 | 5433592 |
| AL045492 | 5433629 |
| AL045500 | 5433637 |
| AL045538 | 5433669 |
| AL045638 | 5433760 |
| AL045977 | 5434077 |
| AL046005 | 5434101 |
| AL046079 | 5434167 |
| AL046107 | 5434194 |
| AL046313 | 5434394 |
| AL046322 | 5434403 |
| AL046640 | 5434705 |
| AL046648 | 5434713 |
| AL046756 | 5434818 |
| AL046802 | 5434864 |
| AL047056 | 5435110 |
| AL047277 | 4727224 |
| AL047334 | 4727905 |
| AL047415 | 4727330 |
| AL047436 | 4727351 |
| AL047535 | 4728531 |
| AL047776 | 4727964 |
| AL047838 | 4728026 |
| AL047850 | 4728038 |
| AL047881 | 4728069 |
| AL048554 | 5936565 |
| AL048699 | 5928420 |
| AL048748 | 5928441 |
| AL048852 | 4728161 |
| AL048890 | 4728199 |
| AL048954 | 4728263 |
| AL049012 | 4728321 |
| AL049145 | 4728455 |
| AL049219 | 4499949 |
| AL049227 | 4499957 |
| AL049229 | 4499961 |
| AL049233 | 4499967 |
| AL049246 | 4499983 |
| AL049252 | 4499993 |
| AL049266 | 4500015 |
| AL049274 | 4500025 |
| AL049276 | 4500029 |
| AL049294 | 4500055 |
| AL049325 | 4500098 |
| AL049367 | 4500158 |
| AL049381 | 4500168 |
| AL049447 | 4500230 |
| AL049470 | 4500264 |
| AL049471 | 4500266 |
| AL049705 | 4678821 |
| AL049784 | 4902698 |
| AL049799 | 4775328 |
| AL049821 | 5791513 |
| AL049932 | 4884176 |
| AL049934 | 4884072 |
| AL049935 | 4884177 |
| AL049941 | 4884184 |
| AL049942 | 4884185 |
| AL049944 | 4884189 |
| AL049951 | 4884198 |
| AL049957 | 4884209 |
| AL049969 | 4884218 |
| AL049974 | 4884224 |
| AL049981 | 4884232 |
| AL049987 | 4884238 |
| AL049996 | 4884248 |
| AL050013 | 4884082 |
| AL050018 | 4884085 |
| AL050035 | 4884276 |
| AL050037 | 4884277 |
| AL050041 | 4884283 |
| AL050051 | 4884099 |
| AL050064 | 4884294 |
| AL050071 | 4884302 |
| AL050073 | 4884305 |
| AL050089 | 4884107 |
| AL050091 | 4884111 |
| AL050100 | 4884127 |
| AL050101 | 4884129 |
| AL050107 | 4884135 |
| AL050136 | 4884346 |
| AL050141 | 4884352 |
| AL050159 | 4884371 |
| AL050161 | 4884375 |
| AL050162 | 4884376 |
| AL050184 | 4884399 |
| AL050186 | 4884401 |
| AL050187 | 4884402 |
| AL050192 | 4884408 |
| AL050197 | 4884434 |
| AL050198 | 4884436 |
| AL050201 | 4884440 |
| AL050204 | 4884443 |
| AL050255 | 4886424 |
| AL050265 | 4886440 |
| AL050268 | 4886442 |
| AL050272 | 4886498 |
| AL050274 | 4886452 |
| AL050282 | 4886464 |
| AL050287 | 4886474 |
| AL050289 | 4886510 |
| AL050290 | 4886512 |
| AL050291 | 4886521 |
| AL050298 | 4886502 |
| AL050355 | 4914575 |
| AL050373 | 4914577 |
| AL079279 | 5102585 |
| AL079288 | 5102747 |
| AL079298 | 5102736 |
| AL079338 | 6522989 |
| AL079529 | 5423423 |
| AL079681 | 5435257 |
| AL079982 | 5435556 |
| AL080059 | 5262463 |
| AL080063 | 5262468 |
| AL080084 | 5262498 |
| AL080088 | 5262504 |
| AL080089 | 5262506 |
| AL080097 | 5262519 |
| AL080118 | 5262548 |
| AL080135 | 5262576 |
| AL080144 | 5262592 |
| AL080172 | 5262642 |
| AL080181 | 5262657 |
| AL080192 | 5262673 |
| AL080201 | 5262685 |
| AL080202 | 5262687 |
| AL080206 | 5262692 |
| AL080209 | 5262698 |
| AL080212 | 5262701 |
| AL080223 | 5262715 |
| AL080224 | 5262716 |
| AL080234 | 5262727 |
| AL096719 | 5419854 |
| AL096857 | 5541862 |
| AL096880 | 5596701 |
| AL109672 | 5689836 |
| AL109728 | 5689794 |
| AL110126 | 5817020 |
| AL11O141 | 5817036 |
| AL110144 | 5817039 |
| AL11O153 | 5817055 |
| AL110159 | 5817063 |
| AL110177 | 5817087 |
| AL110179 | 5817089 |
| AL110180 | 5817090 |
| AL110181 | 5817093 |
| AL110183 | 5817095 |
| AL110185 | 5817098 |
| AL110191 | 5817105 |
| AL110197 | 5817115 |
| AL110206 | 5817125 |
| AL110207 | 5817126 |
| AL11O211 | 5817133 |
| AL110212 | 5817134 |
| AL110214 | 5817136 |
| AL110222 | 5817156 |
| AL110233 | 5817172 |
| AL110238 | 5817180 |
| AL110241 | 5817186 |
| AL110242 | 5817188 |
| AL110256 | 5817216 |
| AL110273 | 5817091 |
| AL110297 | 5817256 |
| AL110357 | 5865965 |
| AL110384 | 5936678 |
| AL110395 | 5866003 |
| AL110412 | 5866020 |
| AL110463 | 5866071 |
| AL117237 | 5834563 |
| AL117340 | 6002147 |
| AL117392 | 5911848 |
| AL117412 | 5912102 |
| AL117413 | 5912103 |
| AL117423 | 5911854 |
| AL117427 | 5911860 |
| AL117429 | 5911863 |
| AL117430 | 5911865 |
| AL117441 | 5911883 |
| AL117443 | 5911887 |
| AL117446 | 5911893 |
| AL117458 | 5911910 |
| AL117471 | 5911940 |
| AL117499 | 5912003 |
| AL117505 | 5912014 |
| AL117514 | 5912027 |
| AL117516 | 5912031 |
| AL117526 | 5912050 |
| AL117534 | 5912062 |
| AL117536 | 5912065 |
| AL117537 | 5912066 |
| AL117543 | 5912075 |
| AL117550 | 5912084 |
| AL117554 | 5912089 |
| AL117563 | 5912112 |
| AL117567 | 5912118 |
| AL117576 | 5912130 |
| AL117584 | 5912144 |
| AL117595 | 5912159 |
| AL117599 | 5912167 |
| AL117609 | 5912183 |
| AL117616 | 5912194 |
| AL117619 | 5912198 |
| AL117621 | 5912202 |
| AL117623 | 5912204 |
| AL117643 | 5912233 |
| AL117644 | 5912234 |
| AL117665 | 5912262 |
| AL117666 | 5912264 |
| AL118582 | 5924481 |
| AL1J8621 | 5924520 |
| AL118746 | 5924645 |
| AL118956 | 5924855 |
| AL118999 | 5924898 |
| AL119319 | 5925218 |
| AL119449 | 5925348 |
| AL119462 | 5925361 |
| AL119562 | 5925461 |
| AL119715 | 5925614 |
| AL119728 | 5925627 |
| AL119859 | 5925758 |
| AL120381 | 5926280 |
| AL120396 | 5926295 |
| AL120521 | 5926420 |
| AL120558 | 5926457 |
| AL120694 | 5926593 |
| AL120741 | 5926641 |
| AL120789 | 5926790 |
| AL120798 | 5926799 |
| AL120807 | 5926808 |
| AL120828 | 5926829 |
| AL120840 | 5926841 |
| AL120972 | 5926973 |
| AL121031 | 5927032 |
| AL121220 | 5927221 |
| AL121262 | 5927263 |
| AL121340 | 5927341 |
| AL121420 | 5927421 |
| AL121423 | 5927424 |
| AL121488 | 5927489 |
| AL121547 | 5927548 |
| AL121603 | 6434634 |
| AL121654 | 6002387 |
| AL121733 | 6012972 |
| AL121735 | 6012990 |
| AL121790 | 8919824 |
| P1122007 | 6635894 |
| AL122063 | 6102855 |
| AL122076 | 6102877 |
| AL122079 | 6102883 |
| AL122084 | 6102892 |
| AL122088 | 6102898 |
| AL122097 | 6102911 |
| AL122121 | 6102948 |
| AL133000 | 6453345 |
| AL133059 | 6453481 |
| AL133074 | 6453517 |
| AL133076 | 6453519 |
| AL133078 | 6453522 |
| AL133080 | 6453526 |
| AL133108 | 6453594 |
| AL133117 | 6453608 |
| AL135879 | 6634068 |
| AP000303 | 4835672 |
| AP000502 | 5926689 |
| AP000527 | 5931505 |
| AP000528 | 5931506 |
| AP000529 | 5931507 |
| AW002185 | 5849101 |
| AW003247 | 5850163 |
| AW003287 | 5850203 |
| AW003442 | 5850358 |
| AW003692 | 5850608 |
| AW003821 | 5850737 |
| AW003952 | 5850868 |
| AW003967 | 5850883 |
| AW005103 | 5853881 |
| AW005947 | 5854725 |
| AW005971 | 5854749 |
| AW006392 | 5855170 |
| AW006468 | 5855246 |
| AW008213 | 5856991 |
| AW008473 | 5857251 |
| AW009562 | 5858340 |
| AW009700 | 5858478 |
| AW009886 | 5858664 |
| AW014145 | 5862902 |
| AW014590 | 5863347 |
| AWOlSOSS | 5863812 |
| AW015094 | 5863851 |
| AW015229 | 5863916 |
| AW015683 | 5864440 |
| AW015828 | 5864585 |
| AW016443 | 5865200 |
| AW016546 | 5865303 |
| AW016611 | 5865368 |
| AW019951 | 5873481 |
| AW020245 | 5873775 |
| AW020429 | 5873959 |
| AW020479 | 5874009 |
| AW020699 | 5874229 |
| AW020820 | 5874350 |
| AW020930 | 5874460 |
| AW020934 | 5874464 |
| AW021402 | 5874932 |
| AW021628 | 5875158 |
| AW021832 | 5875362 |
| AW022017 | 5875557 |
| AW022072 | 5875602 |
| AW022112 | 5875642 |
| AW022300 | 5875830 |
| AW022599 | 5876129 |
| AW022756 | 5876286 |
| AW022792 | 5876322 |
| AW023001 | 5876531 |
| AW023372 | 5876902 |
| AW023413 | 5876943 |
| AW023489 | 5877019 |
| AW023714 | 5877244 |
| AW023884 | 5877344 |
| AW024797 | 5878327 |
| AW025074 | 5878604 |
| AW025130 | 5878660 |
| AW026131 | 5879661 |
| AW026502 | 5880032 |
| AW026543 | 5880073 |
| AW026680 | 5880133 |
| AW027007 | 5885763 |
| AW027219 | 5885975 |
| AW027250 | 5886006 |
| AW027339 | 5886095 |
| AW027586 | 5886342 |
| AW028521 | 5887277 |
| AW028594 | 5887350 |
| AW028606 | 5887362 |
| AW028937 | 5887693 |
| AW029146 | 5887902 |
| AW043908 | 5904437 |
| AW044029 | 5904558 |
| AW044114 | 5904643 |
| AW044142 | 5904671 |
| AW044547 | 5905076 |
| AW051386 | 5913656 |
| AW052140 | 5914499 |
| AW055209 | 5920912 |
| AW057515 | 5933154 |
| AW058559 | 5934198 |
| AW058600 | 5934239 |
| AW062373 | 6013758 |
| AW062376 | 6013761 |
| AW062933 | 6014318 |
| AW067912 | 6022910 |
| AW067913 | 6022911 |
| AW068372 | 6023370 |
| AW068491 | 6023489 |
| AW068579 | 6023577 |
| AW070326 | 6025324 |
| AW071063 | 6026061 |
| AW071696 | 6026694 |
| AW071903 | 6026901 |
| AW071906 | 6026904 |
| AW073731 | 6028729 |
| AW075324 | 6030322 |
| AW075433 | 6030518 |
| AW075906 | 6030904 |
| AW076087 | 6031085 |
| AW078747 | 6033899 |
| AW079132 | 6034284 |
| AW080160 | 6035312 |
| AW080196 | 6035348 |
| AW080456 | 6035608 |
| AW080631 | 6035783 |
| AW080845 | 6035997 |
| AW082236 | 6037388 |
| AW083338 | 6038490 |
| AW083804 | 6038956 |
| AW084017 | 6039169 |
| AW084125 | 6039277 |
| AW084350 | 6039502 |
| AW084663 | 6039815 |
| AW084950 | 6040102 |
| AW085924 | 6041130 |
| AW086063 | 6041310 |
| AW087623 | 6043349 |
| AW088547 | 6044358 |
| AW088608 | 6044425 |
| AW088727 | 6044556 |
| AW090304 | 6047648 |
| AW090544 | 6047888 |
| AW102895 | 6073508 |
| AW102941 | 6073556 |
| AW102951 | 6073566 |
| AW103377 | 6074112 |
| AW103458 | 6074193 |
| AW104561 | 6075296 |
| AW104641 | 6075376 |
| AW105540 | 6076275 |
| AW117546 | 6086130 |
| AW117855 | 6086439 |
| AW118457 | 6087041 |
| AW118595 | 6087179 |
| AW118812 | 6087396 |
| AW129510 | 6117454 |
| AW129657 | 6117601 |
| AW130325 | 6131930 |
| AW130776 | 6132383 |
| AW130969 | 6132576 |
| AW131127 | 6132734 |
| AW131770 | 6133377 |
| AW132009 | 6133616 |
| AW134672 | 6138218 |
| AW134746 | 6138292 |
| AW134772 | 6138318 |
| AW134867 | 6138413 |
| AW135062 | 6138608 |
| AW135187 | 6139408 |
| AW135359 | 6139492 |
| AW135430 | 6139563 |
| AW135666 | 6139799 |
| AW136951 | 6141084 |
| AW138181 | 6142581 |
| AW138413 | 6142731 |
| AW138731 | 6143049 |
| AW139567 | 6144285 |
| AW140049 | 6144767 |
| AW140056 | 6144774 |
| AW148750 | 6196646 |
| AW148884 | 6196780 |
| AW149504 | 6197400 |
| AW149706 | 6197602 |
| AW149817 | 6197713 |
| AW150276 | 6198172 |
| AW150770 | 6198668 |
| AW151254 | 6199152 |
| AW151295 | 6199193 |
| AW151704 | 6199602 |
| AW152107 | 6200005 |
| AW152265 | 6200165 |
| AW152311 | 6200211 |
| AW157047 | 6228448 |
| AW157060 | 6228461 |
| AW157303 | 6228704 |
| AW157674 | 6229075 |
| AW160363 | 6299396 |
| AW160399 | 6299432 |
| AW160447 | 6299480 |
| AW160669 | 6299641 |
| AW160696 | 6299729 |
| AW160731 | 6299764 |
| AW161209 | 6300242 |
| AW161365 | 6300398 |
| AW161467 | 6300500 |
| AW161491 | 6300524 |
| AW161705 | 6300738 |
| AW161853 | 6300886 |
| AW162502 | 6301535 |
| AW163208 | 6302241 |
| AW163314 | 6302347 |
| AW163441 | 6302474 |
| AW163485 | 6302518 |
| AW166338 | 6397863 |
| AW167481 | 6399006 |
| AW167569 | 6399094 |
| AW167579 | 6399104 |
| AW167665 | 6399190 |
| AW168960 | 6400485 |
| AW169156 | 6400681 |
| AW169473 | 6401081 |
| AW169765 | 6401290 |
| AW169876 | 6401401 |
| AW169890 | 6401415 |
| AW169941 | 6401466 |
| AW170193 | 6401707 |
| AW170355 | 6401880 |
| AW170485 | 6402010 |
| AW173161 | 6439109 |
| AW173228 | 6439176 |
| AW173278 | 6439226 |
| AW176020 | 6442070 |
| AW176022 | 6442072 |
| AW176042 | 6442079 |
| AW176062 | 6442099 |
| AW176649 | 6442686 |
| AW176926 | 6442963 |
| AW177204 | 6443228 |
| AW177929 | 6443966 |
| AW177931 | 6443968 |
| AW177936 | 6443973 |
| AW177948 | 6443985 |
| AW177957 | 6443994 |
| AW178342 | 6444379 |
| AW178543 | 6444684 |
| AW178825 | 6444862 |
| AW179006 | 6445043 |
| AW179322 | 6445359 |
| AW181932 | 6450392 |
| AW183601 | 6452115 |
| AW183759 | 6452273 |
| AW183848 | 6452362 |
| AW188391 | 6462827 |
| AW188666 | 6463026 |
| AW235635 | 6568024 |
| C02739 | 1465990 |
| C02912 | 1466163 |
| C03130 | 1466381 |
| C04376 | 1467627 |
| C04910 | 1468161 |
| C04925 | 1468176 |
| C05816 | 1502592 |
| C06196 | 1502972 |
| C06419 | 1503195 |
| C15094 | 1569801 |
| C15322 | 1570029 |
| C16620 | 1571327 |
| C18148 | 1579750 |
| C18181 | 1579783 |
| C18232 | 1579834 |
| C18436 | 1580038 |
| C18484 | 1580086 |
| C18570 | 1580172 |
| C18654 | 1580256 |
| C19105 | 1580707 |
| C20630 | 1621740 |
| C21370 | 1622480 |
| C75042 | 2366104 |
| D00015 | 2618612 |
| D00017 | 219909 |
| D00039 | 3808177 |
| D00422 | 220063 |
| D00860 | 220019 |
| D10040 | 219899 |
| D10495 | 520586 |
| D10924 | 219868 |
| D12135 | 2148331 |
| D12676 | 219702 |
| D13119 | 285909 |
| D13286 | 496368 |
| D13315 | 219663 |
| D13388 | 219587 |
| D13627 | 286010 |
| D13629 | 285984 |
| D13630 | 286000 |
| D13641 | 285986 |
| D13643 | 6630631 |
| D13645 | 286008 |
| D13748 | 219402 |
| D13757 | 219458 |
| D13866 | 433410 |
| D13900 | 433412 |
| D13988 | 285974 |
| D14041 | 2326266 |
| D14043 | 219924 |
| D14446 | 393314 |
| D14530 | 414348 |
| D14657 | 285938 |
| D14658 | 285940 |
| D14659 | 285942 |
| D14662 | 285948 |
| D14664 | 285952 |
| D14665 | 6630617 |
| D14696 | 285962 |
| D14697 | 285964 |
| D14705 | 415305 |
| D14710 | 559324 |
| D14812 | 285968 |
| D14878 | 1435036 |
| D15057 | 493244 |
| D16111 | 435637 |
| D16217 | 303598 |
| D16307 | 303594 |
| D16431 | 598955 |
| D16469 | 758583 |
| D16480 | 493657 |
| D16562 | 506336 |
| D16911 | 598714 |
| D16922 | 598769 |
| D16937 | 598856 |
| D16960 | 598541 |
| D17039 | 598762 |
| D17069 | 598836 |
| D17130 | 598546 |
| D17260 | 598877 |
| D17400 | 451207 |
| D17409 | 2335046 |
| D17554 | 433415 |
| D17652 | 409069 |
| D21092 | 540512 |
| D21163 | 434758 |
| D21209 | 452189 |
| D21210 | 452191 |
| D21243 | 416226 |
| D21260 | 434760 |
| D21262 | 434764 |
| D21851 | 434766 |
| D21853 | 434770 |
| D23662 | 432362 |
| D23672 | 577624 |
| D25215 | 517114 |
| D25274 | 464185 |
| D25283 | 457443 |
| D25328 | 464186 |
| D25542 | 662389 |
| D26068 | 436225 |
| D26129 | 532677 |
| D26362 | 452518 |
| D26485 | 468934 |
| D26488 | 452522 |
| D26598 | 565646 |
| D26600 | 565650 |
| D28118 | 529640 |
| D28358 | 461257 |
| D28420 | 461246 |
| D28473 | 551621 |
| D28476 | 460710 |
| D28480 | 516759 |
| D28482 | 520583 |
| D28540 | 1040688 |
| D28589 | 460714 |
| D29011 | 558525 |
| D29012 | 558527 |
| D29640 | 473930 |
| D29641 | 6633994 |
| D29643 | 473936 |
| D29677 | 473938 |
| D29954 | 473940 |
| D29958 | 473948 |
| D30037 | 1060904 |
| D30655 | 485387 |
| D30921 | 643801 |
| D31763 | 498151 |
| D31764 | 498153 |
| D31767 | 505091 |
| D31770 | 1321631 |
| D31839 | 643590 |
| D31883 | 505093 |
| D31884 | 505095 |
| D31885 | 505097 |
| D31886 | 505099 |
| D31891 | 505109 |
| D32051 | 509032 |
| D37931 | 976228 |
| D37965 | 807818 |
| D37991 | 1019367 |
| D38037 | 532500 |
| D38047 | 1037163 |
| D38073 | 862331 |
| D38293 | 807814 |
| D38305 | 1580723 |
| D38441 | 556513 |
| D38491 | 559327 |
| D38521 | 559329 |
| D38522 | 559331 |
| D38524 | 633070 |
| D38549 | 559702 |
| D38551 | 1531549 |
| D38552 | 559712 |
| D38555 | 559716 |
| D38583 | 560790 |
| D42039 | 577290 |
| D42040 | 577292 |
| D42044 | 577300 |
| D42045 | 577302 |
| D42047 | 577306 |
| D42063 | 924266 |
| D42138 | 1552168 |
| D43947 | 603948 |
| D43948 | 603950 |
| D43950 | 603954 |
| D43951 | 603956 |
| D44466 | 1808577 |
| D44737 | 1572212 |
| D45198 | 971271 |
| D45248 | 1008914 |
| D45370 | 871884 |
| D45887 | 665587 |
| D49372 | 1552240 |
| D49387 | 1088447 |
| D49396 | 682747 |
| D49489 | 1136742 |
| D49490 | 1072306 |
| D50369 | 2605589 |
| D50371 | 2605591 |
| D50372 | 2605593 |
| D50405 | 1665722 |
| D50406 | 3610668 |
| D50420 | 2618577 |
| D50487 | 1742909 |
| D50525 | 1167502 |
| D50579 | 2641989 |
| D50645 | 1741867 |
| D50916 | 1469174 |
| D50919 | 1469180 |
| D50926 | 1469194 |
| D50927 | 6633951 |
| D50931 | 1469204 |
| D50977 | 950730 |
| D52802 | 953038 |
| D53496 | 955393 |
| D54335 | 956232 |
| D55192 | 957089 |
| D55565 | 961356 |
| D55653 | 871882 |
| D55696 | 1890049 |
| D56420 | 971023 |
| D58694 | 968328 |
| D59253 | 1060898 |
| D60110 | 961749 |
| D60970 | 962609 |
| D61285 | 962924 |
| D61391 | 1381026 |
| D63475 | 1665724 |
| D63476 | 1469865 |
| D63477 | 1469867 |
| D63478 | 1469869 |
| D63480 | 1469873 |
| D63486 | 1469885 |
| D63874 | 968887 |
| D63875 | 961441 |
| D63878 | 961447 |
| D63997 | 2662348 |
| D63998 | 974733 |
| D64015 | 2281005 |
| D64110 | 3738221 |
| D67029 | 1669536 |
| D78152 | 1060889 |
| D78275 | 1526425 |
| D78333 | 1655415 |
| D78611 | 1655421 |
| D79205 | 1754620 |
| D79826 | 1180177 |
| D79986 | 1136389 |
| D79991 | 1136397 |
| D79994 | 1136403 |
| D79996 | 1136407 |
| D80001 | 1136417 |
| D80005 | 1136425 |
| D80009 | 1136433 |
| D81635 | 1179512 |
| D81855 | 1180485 |
| D82060 | 1616917 |
| D82149 | 1183573 |
| D82176 | 1183644 |
| D82197 | 1183685 |
| D82303 | 1183700 |
| D82345 | 1841339 |
| D83032 | 1374697 |
| D83077 | 1304131 |
| D83197 | 3893154 |
| D83485 | 1208426 |
| D83703 | 1747315 |
| D83735 | 1526431 |
| D83780 | 1228042 |
| D84105 | 1256700 |
| D84145 | 2114143 |
| D84476 | 1805499 |
| D84557 | 1944481 |
| D85181 | 1906795 |
| D85758 | 1374694 |
| D85777 | 1747323 |
| D86228 | 2081621 |
| D86322 | 2467376 |
| D86326 | 2988343 |
| D86967 | 1504007 |
| D86971 | 1504015 |
| D86972 | 1504017 |
| D86974 | 1504021 |
| D86984 | 1504041 |
| D86985 | 6634002 |
| D87078 | 6634004 |
| D87127 | 1817551 |
| D87292 | 1877030 |
| D87328 | 1813423 |
| D87437 | 1665768 |
| D87438 | 2055294 |
| D87442 | 1665772 |
| D87443 | 1665774 |
| D87444 | 1665776 |
| D87450 | 1665788 |
| D87452 | 1665792 |
| D87453 | 1665794 |
| D87455 | 1665798 |
| D87462 | 1665808 |
| D87466 | 1665816 |
| D87666 | 1620016 |
| D87667 | 1620019 |
| D87682 | 1663699 |
| D87684 | 1663703 |
| D87685 | 1663705 |
| D87735 | 1620021 |
| D87742 | 1665824 |
| D87845 | 1765863 |
| D87930 | 2443337 |
| D87953 | 1596166 |
| D87969 | 1694636 |
| D88153 | 2289785 |
| D88208 | 4586400 |
| D88532 | 1661000 |
| D88674 | 2641951 |
| D89053 | 4165017 |
| D89077 | 1694681 |
| D89092 | 2780747 |
| D89289 | 2055306 |
| D89667 | 1731808 |
| D89675 | 2055308 |
| D89678 | 3218539 |
| D89937 | 3184392 |
| D90209 | 220087 |
| D90226 | 219946 |
| D90228 | 219917 |
| D90359 | 559319 |
| D90373 | 219477 |
| D90427 | 220150 |
| D90452 | 219895 |
| E00195 | 2168491 |
| E00882 | 2169143 |
| E01497 | 2169753 |
| E01500 | 2169756 |
| E01574 | 2169827 |
| E01650 | 2169903 |
| E01888 | 2170137 |
| E01915 | 2170164 |
| E01932 | 2170180 |
| E01954 | 2170202 |
| E01956 | 2170204 |
| E01979 | 2170227 |
| E02135 | 2170373 |
| E02628 | 2170856 |
| E02822 | 2171050 |
| E02823 | 2171051 |
| E03413 | 2171629 |
| E03414 | 2171630 |
| E03569 | 2171785 |
| E03814 | 2172028 |
| E05692 | 2173879 |
| E05957 | 2174144 |
| E06721 | 2174903 |
| E07798 | 2175931 |
| E08293 | 2176413 |
| E08515 | 2176630 |
| E08663 | 2176776 |
| E08764 | 2176876 |
| F01265 | 708357 |
| F01327 | 707344 |
| F02595 | 646152 |
| F05508 | 669324 |
| F05894 | 669710 |
| F06621 | 672219 |
| F06739 | 672353 |
| F07019 | 672659 |
| F07432 | 673092 |
| F07635 | 673321 |
| F07647 | 673334 |
| F08493 | 677060 |
| F08579 | 677146 |
| F09972 | 682505 |
| F12000 | 706326 |
| F12493 | 708486 |
| F12883 | 708906 |
| F13179 | 709211 |
| F13272 | 709376 |
| F19390 | 4825701 |
| F22763 | 2061939 |
| F24428 | 4810054 |
| F25339 | 4810965 |
| F27229 | 4812855 |
| F27302 | 4812928 |
| F27796 | 4813422 |
| F28190 | 4813816 |
| F28764 | 4814390 |
| F33993 | 4819619 |
| F34889 | 4820515 |
| G16768 | 1214194 |
| H00112 | 863045 |
| H00742 | 863675 |
| H00752 | 863685 |
| H00817 | 863750 |
| H01129 | 864062 |
| H01205 | 864138 |
| H01332 | 864265 |
| H01419 | 864352 |
| H01516 | 864449 |
| H01539 | 864472 |
| H01926 | 864859 |
| H01979 | 864912 |
| H02039 | 864972 |
| H02158 | 865091 |
| H02230 | 865163 |
| H02231 | 865164 |
| H02294 | 865227 |
| H02307 | 865240 |
| H02308 | 865241 |
| H02550 | 865483 |
| H02725 | 865658 |
| H02837 | 865770 |
| H03146 | 866079 |
| H03436 | 866369 |
| H03650 | 866583 |
| H03785 | 866718 |
| H03882 | 866815 |
| H04072 | 867005 |
| H04382 | 867315 |
| H04757 | 868309 |
| H04789 | 868341 |
| H04828 | 868380 |
| H05085 | 868637 |
| H05140 | 868692 |
| H05264 | 868816 |
| H05325 | 868877 |
| H05326 | 868878 |
| H05445 | 868997 |
| H05563 | 869115 |
| H05626 | 869178 |
| H05645 | 869197 |
| H05706 | 869258 |
| H05734 | 869286 |
| H05741 | 869293 |
| H05768 | 869320 |
| H05777 | 869329 |
| H05818 | 869370 |
| H05961 | 869513 |
| H06040 | 869592 |
| H06093 | 869645 |
| H06113 | 869665 |
| H06154 | 869706 |
| H06249 | 869801 |
| H06313 | 869865 |
| H06377 | 869929 |
| H06380 | 869932 |
| H06525 | 870057 |
| H06621 | 870153 |
| H06944 | 870476 |
| H07920 | 872742 |
| H07934 | 872756 |
| H08016 | 872838 |
| H08120 | 872942 |
| H08194 | 873016 |
| H08210 | 873032 |
| H08227 | 873049 |
| H08397 | 873219 |
| H08417 | 873239 |
| H08424 | 873246 |
| H08511 | 873333 |
| H08542 | 873364 |
| H08548 | 873370 |
| H08560 | 873382 |
| H08582 | 873404 |
| H08595 | 873417 |
| H08598 | 873420 |
| H08621 | 873443 |
| H08725 | 873547 |
| H08785 | 873607 |
| H08796 | 873618 |
| H08820 | 873642 |
| H08850 | 873672 |
| H08862 | 873684 |
| H08899 | 873721 |
| H08924 | 873746 |
| H08929 | 873751 |
| H08959 | 873781 |
| H09065 | 873887 |
| H09076 | 873898 |
| H09132 | 873954 |
| H09164 | 873986 |
| H09172 | 873994 |
| H09222 | 874044 |
| H09332 | 874154 |
| H09529 | 874351 |
| H09541 | 874363 |
| H09616 | 874438 |
| H09642 | 874464 |
| H09747 | 874569 |
| H09749 | 874571 |
| H09774 | 874596 |
| H09818 | 874640 |
| H09882 | 874704 |
| H09936 | 874758 |
| H09940 | 874762 |
| H09959 | 874781 |
| H09966 | 874788 |
| H09996 | 874818 |
| H09997 | 874819 |
| H10011 | 874833 |
| H10028 | 874850 |
| H10036 | 874858 |
| H10068 | 874890 |
| H10072 | 874894 |
| H10073 | 874895 |
| H10107 | 874929 |
| H10192 | 875014 |
| H10208 | 875030 |
| H10228 | 875050 |
| H10231 | 875053 |
| H10342 | 875164 |
| H10344 | 875166 |
| H10404 | 875226 |
| H10413 | 875235 |
| H10429 | 875251 |
| H10482 | 875304 |
| H10555 | 875377 |
| H10665 | 875487 |
| H10713 | 875564 |
| H10761 | 875581 |
| H10778 | 875598 |
| H10788 | 875608 |
| H10932 | 875752 |
| H10981 | 875801 |
| H11036 | 875856 |
| H11051 | 875871 |
| H11063 | 875883 |
| H11071 | 875891 |
| H11376 | 876196 |
| H11433 | 876253 |
| H11519 | 876339 |
| H11631 | 876451 |
| H11642 | 876462 |
| H11678 | 876498 |
| H11730 | 876550 |
| H11732 | 876552 |
| H11760 | 876580 |
| H11792 | 876612 |
| H12056 | 876876 |
| H12081 | 876901 |
| H12090 | 876910 |
| H12109 | 876929 |
| H12190 | 877010 |
| H12229 | 877049 |
| H12254 | 877074 |
| H12338 | 877158 |
| H12519 | 877339 |
| H12527 | 877347 |
| H12923 | 877743 |
| H12981 | 877801 |
| H13008 | 877828 |
| H13339 | 878159 |
| H13406 | 878226 |
| H13438 | 878258 |
| H13688 | 878508 |
| H13738 | 878558 |
| H13891 | 878711 |
| H14057 | 878905 |
| H14446 | 879266 |
| H14949 | 879769 |
| H14985 | 879805 |
| H15040 | 879860 |
| H15050 | 879870 |
| H15099 | 879919 |
| H15104 | 879924 |
| H15250 | 880070 |
| H15366 | 880186 |
| H15408 | 880228 |
| H15417 | 880237 |
| H15533 | 880353 |
| H15549 | 880369 |
| H15654 | 880474 |
| H15655 | 880475 |
| H5660 | 880480 |
| H15675 | 880495 |
| H15677 | 880497 |
| H15695 | 880515 |
| H15696 | 880516 |
| H15746 | 880566 |
| H15910 | 880730 |
| H15926 | 880746 |
| H16152 | 880972 |
| H16224 | 881044 |
| H16439 | 881259 |
| H16454 | 881274 |
| H16455 | 881275 |
| H16537 | 882762 |
| H16554 | 882779 |
| H16600 | 882840 |
| H16743 | 882983 |
| H16772 | 883012 |
| H16803 | 883043 |
| H16833 | 883073 |
| H16851 | 883091 |
| H16903 | 883143 |
| H17002 | 883242 |
| H17022 | 883262 |
| H17038 | 883278 |
| H17121 | 883361 |
| H17139 | 883379 |
| H17170 | 883410 |
| H17195 | 883435 |
| H17363 | 883603 |
| H17516 | 883756 |
| H17618 | 883858 |
| H17626 | 883866 |
| H17630 | 883870 |
| H17696 | 883936 |
| H17741 | 883981 |
| H17763 | 884003 |
| H17773 | 884013 |
| H17800 | 884040 |
| H17804 | 884044 |
| H17869 | 884109 |
| H17871 | 884111 |
| H17882 | 884122 |
| H17888 | 884128 |
| H17919 | 884159 |
| H17921 | 884161 |
| H17934 | 884174 |
| H17943 | 884183 |
| H18015 | 884255 |
| H18017 | 884257 |
| H18068 | 884308 |
| H18423 | 884663 |
| H18424 | 884664 |
| 818433 | 884673 |
| 818471 | 884711 |
| 818560 | 884800 |
| H18626 | 884866 |
| H18630 | 884870 |
| H18633 | 884873 |
| H18726 | 884966 |
| H18732 | 884972 |
| H18838 | 885078 |
| H18924 | 885164 |
| H18932 | 885172 |
| H18936 | 885176 |
| H19068 | 885308 |
| H19128 | 885368 |
| H19153 | 885393 |
| H19217 | 885457 |
| H19234 | 885474 |
| H19242 | 885482 |
| H19320 | 885560 |
| H19324 | 885564 |
| H19327 | 885567 |
| H19367 | 888062 |
| H19393 | 888088 |
| H19427 | 888122 |
| H19446 | 888141 |
| H19826 | 888521 |
| H20046 | 888741 |
| H20086 | 888781 |
| H20388 | 889083 |
| H20547 | 889242 |
| H20717 | 889412 |
| H20743 | 889438 |
| H20744 | 889439 |
| H20747 | 889442 |
| H20847 | 889542 |
| H20888 | 889583 |
| H21943 | 890638 |
| H22136 | 890831 |
| H22563 | 891258 |
| H22568 | 891263 |
| H22734 | 891429 |
| H22824 | 891519 |
| H22853 | 891548 |
| H22854 | 891549 |
| H22928 | 891623 |
| H22932 | 891627 |
| H22956 | 891651 |
| H23049 | 891744 |
| H23137 | 891832 |
| H23157 | 891852 |
| H23187 | 891882 |
| H23202 | 891897 |
| H23211 | 891906 |
| H23232 | 891927 |
| H23277 | 891972 |
| H23370 | 892065 |
| H23422 | 892117 |
| H23459 | 892154 |
| H23482 | 892177 |
| H23524 | 892219 |
| H23548 | 892243 |
| H23555 | 892250 |
| H23837 | 892532 |
| H23983 | 892678 |
| H23985 | 892680 |
| H24006 | 892701 |
| H24011 | 892706 |
| H24176 | 892871 |
| H24313 | 893008 |
| H24316 | 893011 |
| H24317 | 893012 |
| H24329 | 893024 |
| H24350 | 893045 |
| H24356 | 893051 |
| H24580 | 893479 |
| H24856 | 893755 |
| H24954 | 893853 |
| H25019 | 893918 |
| H25211 | 894334 |
| H25546 | 894669 |
| H25689 | 894812 |
| H25806 | 894929 |
| H25825 | 894948 |
| H25880 | 895003 |
| H26319 | 895442 |
| H26426 | 895549 |
| H26492 | 1146489 |
| H26678 | 896668 |
| H27379 | 897369 |
| H27545 | 897535 |
| H27986 | 898339 |
| H28091 | 898444 |
| H28581 | 898934 |
| H28710 | 899664 |
| H28973 | 899883 |
| H29149 | 900059 |
| H29198 | 900108 |
| H29207 | 900117 |
| H29215 | 900125 |
| H29216 | 900126 |
| H29227 | 900137 |
| H29256 | 900166 |
| H29268 | 900178 |
| H29290 | 900200 |
| H29315 | 900225 |
| H29499 | 900409 |
| H29500 | 900410 |
| H29521 | 900431 |
| H29635 | 900545 |
| H29664 | 900574 |
| H29897 | 900807 |
| H30141 | 901051 |
| H37817 | 907316 |
| H38210 | 907709 |
| H38263 | 907762 |
| H38425 | 907924 |
| H38564 | 908063 |
| H38623 | 908122 |
| H38650 | 908149 |
| H38848 | 908347 |
| H38880 | 908379 |
| H38914 | 908413 |
| H39560 | 915612 |
| H39809 | 915861 |
| H39906 | 915958 |
| H39960 | 916012 |
| H40350 | 916402 |
| H40738 | 916790 |
| H40921 | 916973 |
| H41203 | 917255 |
| H41222 | 917274 |
| H41489 | 917541 |
| H42679 | 918731 |
| H43317 | 919369 |
| H43707 | 919759 |
| H44032 | 920084 |
| H44448 | 920500 |
| H44861 | 920913 |
| H45289 | 921341 |
| H45447 | 921499 |
| H45550 | 921602 |
| H45711 | 921763 |
| H45810 | 921862 |
| H46176 | 922228 |
| H47015 | 923067 |
| H47076 | 923128 |
| H47089 | 923141 |
| H47252 | 923304 |
| H47315 | 923367 |
| H47325 | 923377 |
| H47363 | 923415 |
| H47715 | 923767 |
| H48050 | 924102 |
| H46070 | 924122 |
| H48138 | 924190 |
| H48257 | 986644 |
| H48278 | 986665 |
| H48318 | 986705 |
| H48472 | 986859 |
| H48502 | 988342 |
| H48697 | 988537 |
| H49053 | 988894 |
| H49329 | 989170 |
| H49331 | 989172 |
| H49519 | 989360 |
| H49828 | 989669 |
| H49903 | 989744 |
| H50086 | 989927 |
| H50566 | 990407 |
| H50655 | 990496 |
| H50656 | 990497 |
| H50677 | 990518 |
| H50770 | 990611 |
| H51039 | 990880 |
| H51122 | 990963 |
| H51425 | 991266 |
| H51549 | 991390 |
| H51749 | 991590 |
| H51765 | 991606 |
| H51866 | 991707 |
| H51914 | 991755 |
| H51976 | 991817 |
| H52001 | 991842 |
| H52048 | 991889 |
| H52119 | 991960 |
| H52247 | 992088 |
| H52361 | 992202 |
| H52446 | 992287 |
| H55503 | 992344 |
| H53141 | 993288 |
| H53340 | 993487 |
| H53964 | 994111 |
| H54020 | 994167 |
| H54240 | 994387 |
| H54364 | 994511 |
| H54417 | 994564 |
| H54451 | 994598 |
| H54628 | 994995 |
| H54659 | 995026 |
| H54680 | 995047 |
| H54752 | 995172 |
| H55907 | 1004551 |
| H55915 | 1004559 |
| H55920 | 1004564 |
| H55955 | 1004599 |
| H56028 | 1004672 |
| H56453 | 1005097 |
| H56898 | 1009730 |
| H56903 | 1009735 |
| H57060 | 1009892 |
| H57064 | 1009896 |
| H57082 | 1009914 |
| H57135 | 1009967 |
| H57136 | 1009968 |
| H57382 | 1010214 |
| H57485 | 1010317 |
| H57585 | 1010417 |
| H57648 | 1010480 |
| H57857 | 1010689 |
| H58234 | 1011066 |
| H58534 | 1011366 |
| H58542 | 1011374 |
| H58571 | 1011403 |
| H58702 | 1011534 |
| H58834 | 1011666 |
| H59188 | 1012020 |
| H59203 | 1012035 |
| H59305 | 1012137 |
| H59731 | 1012563 |
| H59791 | 1012623 |
| H60247 | 1013079 |
| H60344 | 1013176 |
| H60581 | 1013413 |
| H60696 | 1013528 |
| H61016 | 1013848 |
| H61082 | 1013914 |
| H61193 | 1014025 |
| H61232 | 1014064 |
| H61552 | 1014384 |
| H61758 | 1014590 |
| H61886 | 1014718 |
| H62162 | 1014994 |
| H62529 | 1016875 |
| H62563 | 1016909 |
| H62839 | 1017185 |
| H63175 | 1017976 |
| H63223 | 1018024 |
| H63416 | 1018217 |
| H63668 | 1018469 |
| H63865 | 1018666 |
| H63952 | 1018753 |
| H64095 | 1018896 |
| H64252 | 1022992 |
| H64556 | 1114343 |
| H64719 | 1023459 |
| H65029 | 1023769 |
| H65030 | 1023770 |
| H65286 | 1024026 |
| H65300 | 1024040 |
| H65395 | 1024135 |
| H65423 | 1024163 |
| H65659 | 1024399 |
| H66398 | 1025138 |
| H66675 | 1025415 |
| H66840 | 1025580 |
| H66877 | 1025617 |
| H67292 | 1026032 |
| H67712 | 1026452 |
| H67975 | 1026715 |
| H67988 | 1026728 |
| H68312 | 1027052 |
| H68542 | 1027282 |
| H68655 | 1027395 |
| H68845 | 1030355 |
| H68848 | 1030358 |
| H68988 | 1030214 |
| H69070 | 1030320 |
| H69529 | 1039735 |
| H69553 | 1039759 |
| H69691 | 1039897 |
| H70047 | 1114777 |
| H70269 | 1040475 |
| H70543 | 1042310 |
| H70670 | 1042486 |
| H70775 | 1042591 |
| H70942 | 1042758 |
| H71076 | 1042892 |
| H71230 | 1043046 |
| H71242 | 1043058 |
| H71713 | 1043529 |
| H72093 | 1043909 |
| H72107 | 1043923 |
| H72322 | 1044138 |
| H72591 | 1044407 |
| H72643 | 1044459 |
| H72722 | 1044538 |
| H72937 | 1044753 |
| H72959 | 1044775 |
| H73013 | 1046553 |
| H73241 | 1047389 |
| H73335 | 1047097 |
| H73387 | 1047099 |
| H73590 | 1046649 |
| H73806 | 1046740 |
| H73947 | 1047015 |
| H74086 | 1047298 |
| H74119 | 1047331 |
| H74265 | 1047611 |
| H75328 | 1049820 |
| H75459 | 1050105 |
| H75599 | 1049527 |
| H75695 | 1049638 |
| H75860 | 1049931 |
| H77297 | 1055386 |
| H77494 | 1055583 |
| H77706 | 1055795 |
| H77727 | 1055816 |
| H77766 | 1055855 |
| H78097 | 1056186 |
| H78134 | 1056223 |
| H78135 | 1056224 |
| H78478 | 1056567 |
| H78517 | 1056606 |
| H78888 | 1056977 |
| H78954 | 1057043 |
| H79035 | 1057124 |
| H79078 | 1057167 |
| H79449 | 1057538 |
| H79466 | 1057555 |
| H79534 | 1057623 |
| H79566 | 1057655 |
| H79639 | 1057728 |
| H79973 | 1058062 |
| H80063 | 1058152 |
| H80215 | 1058304 |
| H80325 | 1058414 |
| H80685 | 1058774 |
| H80853 | 1058942 |
| H80936 | 1059025 |
| H81543 | 1114633 |
| H81554 | 1059643 |
| H81659 | 1059748 |
| H81716 | 1059805 |
| H81755 | 1059844 |
| H81817 | 1059906 |
| H82081 | 1060170 |
| H82380 | 1060469 |
| H82596 | 1060685 |
| H82706 | 1060795 |
| H83215 | 1061885 |
| H83283 | 1061953 |
| H83317 | 1061987 |
| H83328 | 1061998 |
| H83524 | 1062195 |
| H83574 | 1062245 |
| H83781 | 1062452 |
| H83784 | 1062455 |
| H84287 | 1062958 |
| H84369 | 1063040 |
| H84657 | 1063892 |
| H84670 | 1063794 |
| H84729 | 1063923 |
| H84734 | 1063928 |
| H84759 | 1064067 |
| H84871 | 1064170 |
| H84915 | 1064410 |
| H84926 | 1064421 |
| H85200 | 1064009 |
| H85345 | 1064319 |
| H85437 | 1064459 |
| H85464 | 1064486 |
| H85806 | 1067385 |
| H86010 | 1067589 |
| H86275 | 1067854 |
| H86518 | 1068097 |
| H86554 | 1068133 |
| H86559 | 1068138 |
| H86816 | 1068395 |
| H87106 | 1068685 |
| H87143 | 1068722 |
| H87175 | 1068754 |
| H87419 | 1068998 |
| H87420 | 1068999 |
| H87471 | 1069050 |
| H87807 | 1069386 |
| H88143 | 1069722 |
| H88261 | 1069840 |
| H88329 | 1069908 |
| H88334 | 1069913 |
| H88424 | 1070684 |
| H88485 | 1070745 |
| H88588 | 1070848 |
| H89036 | 1071296 |
| H89104 | 1071364 |
| H89330 | 1071590 |
| H89331 | 1115008 |
| H89376 | 1071636 |
| H89563 | 1115032 |
| H89582 | 1115036 |
| H89589 | 1115038 |
| H89698 | 1080128 |
| H89713 | 1080143 |
| H89795 | 1080225 |
| H89883 | 1080313 |
| H91011 | 1081441 |
| H91641 | 1087219 |
| H91680 | 1087258 |
| H91721 | 1087299 |
| H92106 | 1087684 |
| H92215 | 1087793 |
| H92216 | 1087794 |
| H92234 | 1087812 |
| H92525 | 1088103 |
| H92683 | 1099011 |
| H92821 | 1099149 |
| H92875 | 1099203 |
| H93191 | 1099519 |
| H93264 | 1099592 |
| H93328 | 1099656 |
| H93335 | 1099663 |
| H93381 | 1099709 |
| H93424 | 1099752 |
| H93552 | 1099880 |
| H93650 | 1099978 |
| H94179 | 1101475 |
| H94262 | 1101558 |
| H94409 | 1102042 |
| H94471 | 1102104 |
| H94482 | 1102115 |
| H94563 | 1102196 |
| H94617 | 1102250 |
| H94720 | 1102353 |
| H94870 | 1102503 |
| H94927 | 1102560 |
| H94944 | 1102577 |
| H95088 | 1102721 |
| H95161 | 1102794 |
| H95535 | 1108677 |
| H95633 | 1108775 |
| H95638 | 1108780 |
| H95712 | 1108854 |
| H95976 | 1109118 |
| H95978 | 1109120 |
| H96053 | 1109195 |
| H96356 | 1109498 |
| H96392 | 1109534 |
| H96504 | 1109972 |
| H96527 | 1110013 |
| H96557 | 1110043 |
| H96605 | 1110091 |
| H96643 | 1110129 |
| H96647 | 1110133 |
| H96738 | 1110224 |
| H96904 | 1110390 |
| H96908 | 1110394 |
| H96926 | 1113969 |
| H97000 | 1114043 |
| H97033 | 1114076 |
| H97090 | 1114133 |
| H97140 | 1114183 |
| H97186 | 1114229 |
| H97488 | 1118373 |
| H97508 | 1118393 |
| H97514 | 1118399 |
| H97565 | 1118450 |
| H97778 | 1118663 |
| H97851 | 1118736 |
| H97880 | 1118765 |
| H97989 | 1118874 |
| H97993 | 1118878 |
| H98218 | 1119103 |
| H98244 | 1119129 |
| H98255 | 1119140 |
| H98655 | 1123323 |
| H98676 | 1123344 |
| H98688 | 1123356 |
| H98694 | 1123362 |
| H98756 | 1123424 |
| H98812 | 1123480 |
| H98822 | 1123490 |
| H99035 | 1123703 |
| H99075 | 1123743 |
| H99215 | 1123883 |
| H99333 | 1124001 |
| H99460 | 1124128 |
| H99586 | 1124254 |
| H99611 | 1124279 |
| H99650 | 1124318 |
| H99672 | 1124340 |
| H99694 | 1124362 |
| H99722 | 1124390 |
| H99766 | 1124434 |
| H99813 | 1124481 |
| H99997 | 1124665 |
| J00194 | 188231 |
| J00200 | 188411 |
| J02642 | 182862 |
| J02853 | 598146 |
| J02871 | 180968 |
| J02876 | 182413 |
| J02888 | 190817 |
| J02908 | 178854 |
| J02923 | 189501 |
| J02959 | 187174 |
| J02966 | 339919 |
| J03007 | 179211 |
| J03015 | 337755 |
| J03040 | 338312 |
| J03143 | 184650 |
| J03171 | 184645 |
| J03209 | 188618 |
| J03248 | 183053 |
| J03250 | 339805 |
| J03464 | 179595 |
| J03473 | 337423 |
| J03503 | 189765 |
| J03507 | 179715 |
| J03537 | 337513 |
| J03544 | 187596 |
| J03575 | 189737 |
| J03592 | 339722 |
| J03746 | 183655 |
| J03779 | 179833 |
| J03799 | 186840 |
| J03802 | 190717 |
| J03827 | 340418 |
| J03870 | 337751 |
| J03934 | 189245 |
| J04080 | 179645 |
| J04137 | 177782 |
| J04162 | 183036 |
| J04164 | 177801 |
| J04177 | 179729 |
| J04183 | 186929 |
| J04205 | 178686 |
| J04208 | 186391 |
| J04443 | 551175 |
| J04478 | 179697 |
| J04607 | 339666 |
| J04611 | 178649 |
| J04615 | 338246 |
| J04621 | 184428 |
| J04759 | 190280 |
| J04794 | 178480 |
| J04823 | 1311703 |
| J04970 | 5809681 |
| J04973 | 180927 |
| J05021 | 340216 |
| J05032 | 179101 |
| J05176 | 177932 |
| J05192 | 178026 |
| J05211 | 181607 |
| J05480 | 179807 |
| J05533 | 189856 |
| J05594 | 1203981 |
| J05633 | 186504 |
| K00409 | 188523 |
| K00558 | 340020 |
| K01144 | 188469 |
| K01911 | 189273 |
| K02765 | 179664 |
| K03002 | 180468 |
| K03515 | 189237 |
| L00160 | 189904 |
| L00635 | 292032 |
| L01042 | 184097 |
| LO1100 | 184507 |
| L01439 | 183358 |
| L02426 | 403455 |
| L02547 | 180598 |
| L03558 | 291926 |
| L04636 | 338044 |
| L05091 | 388030 |
| L05092 | 388031 |
| L05093 | 401844 |
| L05186 | 182394 |
| L05425 | 179284 |
| L05779 | 181394 |
| L06070 | 292509 |
| L06132 | 340198 |
| L06133 | 179252 |
| L06850 | 180116 |
| L07033 | 184502 |
| L07077 | 452044 |
| L07261 | 178087 |
| L07515 | 184310 |
| L07517 | 292043 |
| L07540 | 190153 |
| L07633 | 186512 |
| L07758 | 177764 |
| L08044 | 307520 |
| L08437 | 179291 |
| L08441 | 179295 |
| L08599 | 340184 |
| L09159 | 307374 |
| L09235 | 291867 |
| L09749 | 339906 |
| L10284 | 186522 |
| L10320 | 182310 |
| L10376 | 307378 |
| L10678 | 190387 |
| L10910 | 405191 |
| L11005 | 438655 |
| L11066 | 307322 |
| L11284 | 307183 |
| L11566 | 337492 |
| L11667 | 348909 |
| L11932 | 307423 |
| L12136 | 181536 |
| L12168 | 178083 |
| L12387 | 459835 |
| L12686 | 349765 |
| L13210 | 307152 |
| L13385 | 349823 |
| L13434 | 291843 |
| L13773 | 306446 |
| L13799 | 306548 |
| L13806 | 306554 |
| L13848 | 307382 |
| L13852 | 520832 |
| L13977 | 431320 |
| L14778 | 306476 |
| L14837 | 292937 |
| L14848 | 508491 |
| L15203 | 402482 |
| L15702 | 291921 |
| L16510 | 291887 |
| L16558 | 307387 |
| L16785 | 349475 |
| L17128 | 1220308 |
| L19161 | 306899 |
| L19184 | 440305 |
| L19185 | 440307 |
| L19437 | 4995970 |
| L19597 | 306467 |
| L19605 | 457128 |
| L19713 | 347318 |
| L19779 | 306828 |
| L19872 | 416141 |
| L19956 | 306456 |
| L20010 | 306831 |
| L20422 | 437362 |
| L20431 | 398028 |
| L20688 | 404044 |
| L20773 | 431308 |
| L20859 | 306769 |
| L20941 | 507251 |
| L22009 | 347313 |
| L22569 | 348706 |
| L23320 | 410217 |
| L25081 | 407698 |
| L25085 | 459833 |
| L25610 | 425142 |
| L25899 | 414586 |
| L26081 | 799328 |
| L27841 | 450276 |
| L28010 | 452047 |
| L28997 | 607027 |
| L29008 | 496077 |
| L29158 | 465157 |
| L31801 | 561721 |
| L31951 | 598182 |
| L33404 | 521214 |
| L33930 | 500848 |
| L34087 | 619817 |
| L34839 | 1220373 |
| L34840 | 2766555 |
| L35249 | 522192 |
| L36642 | 551607 |
| L37080 | 559045 |
| L38486 | 790816 |
| L38608 | 886257 |
| L38941 | 1008855 |
| L38951 | 893287 |
| L38961 | 624703 |
| L38995 | 704415 |
| L39833 | 666896 |
| L40403 | 887377 |
| L41143 | 736684 |
| L42110 | 904118 |
| L42531 | 886283 |
| L42542 | 974142 |
| L47276 | 986910 |
| L47345 | 992562 |
| L47647 | 1000861 |
| L47665 | 1088278 |
| L48984 | 1066728 |
| L49115 | 1369830 |
| L49399 | 1381109 |
| L49504 | 1236232 |
| L77701 | 1280205 |
| L77964 | 1294778 |
| M10036 | 339840 |
| M10119 | 182517 |
| M10905 | 182696 |
| M10906 | 337747 |
| M10941 | 180422 |
| M11058 | 184243 |
| M11119 | 182205 |
| M11147 | 182513 |
| M11233 | 181179 |
| M11353 | 184092 |
| M11548 | 340238 |
| M11560 | 178350 |
| M11887 | 184157 |
| M11949 | 190687 |
| M12125 | 339951 |
| M12623 | 184233 |
| M12670 | 182482 |
| M12938 | 182515 |
| M13231 | 339168 |
| M13520 | 179459 |
| M13692 | 178256 |
| M14200 | 181477 |
| M14328 | 182113 |
| M14335 | 182797 |
| M14631 | 183416 |
| M14648 | 340306 |
| M15178 | 184199 |
| M15395 | 186933 |
| M15470 | 187680 |
| M15661 | 337577 |
| M15796 | 181271 |
| M15885 | 338414 |
| M15887 | 181960 |
| M15990 | 181267 |
| M16247 | 178044 |
| M16342 | 184266 |
| M16553 | 339503 |
| M16660 | 184420 |
| M16768 | 339399 |
| M16804 | 339408 |
| M16827 | 177963 |
| M16942 | 188352 |
| M17254 | 182186 |
| M17323 | 2072750 |
| M17324 | 2072751 |
| M17325 | 2072752 |
| M17517 | 180497 |
| M17733 | 339688 |
| M17851 | 182860 |
| M17885 | 190231 |
| M17886 | 190233 |
| M18366 | 179131 |
| M19308 | 339782 |
| M19961 | 180940 |
| M20030 | 338422 |
| M20260 | 178891 |
| M20372 | 189372 |
| M20471 | 179396 |
| M20681 | 183684 |
| M21154 | 178517 |
| M21302 | 338424 |
| M21574 | 189733 |
| M21895 | 189523 |
| M21896 | 189525 |
| M21897 | 189529 |
| M22324 | 178535 |
| M22382 | 190126 |
| M22414 | 186260 |
| M22538 | 986883 |
| M22590 | 179418 |
| M22865 | 181226 |
| M22918 | 189019 |
| M22920 | 189021 |
| M22976 | 181228 |
| M23161 | 339899 |
| M23613 | 189271 |
| M24194 | 187701 |
| M24594 | 186262 |
| M24630 | 514363 |
| M24795 | 178670 |
| M24902 | 189618 |
| M24906 | 537531 |
| M25077 | 387656 |
| M25246 | 340233 |
| M25756 | 338050 |
| M26324 | 186701 |
| M26325 | 186688 |
| M26383 | 188627 |
| M26481 | 619789 |
| M26663 | 618463 |
| M26880 | 340067 |
| M27110 | 190084 |
| M27332 | 540460 |
| M27334 | 540463 |
| M27335 | 540465 |
| M27504 | 339809 |
| M27544 | 184829 |
| M27689 | 188431 |
| M27691 | 181038 |
| M27826 | 804763 |
| M27937 | 187507 |
| M28016 | 337203 |
| M28211 | 550067 |
| M28372 | 643575 |
| M28526 | 189775 |
| M29064 | 337452 |
| M29366 | 181979 |
| M29536 | 182066 |
| M29548 | 181966 |
| M29550 | 180706 |
| M29870 | 190823 |
| M29872 | 178131 |
| M30448 | 181154 |
| M30818 | 188902 |
| M30938 | 186793 |
| M31212 | 188589 |
| M31606 | 189940 |
| M31627 | 184485 |
| M31630 | 183787 |
| M31899 | 182178 |
| M32790 | 180804 |
| M33146 | 181070 |
| M33197 | 182976 |
| M33308 | 340236 |
| M34175 | 179332 |
| M34181 | 189982 |
| M34840 | 189620 |
| M35252 | 180925 |
| M36072 | 337494 |
| M36341 | 178984 |
| M36634 | 340264 |
| M36647 | 188564 |
| M37197 | 179968 |
| M37400 | 179066 |
| M37583 | 184059 |
| M37716 | 338266 |
| M38188 | 189378 |
| M38690 | 1048988 |
| M55265 | 177993 |
| M55409 | 189596 |
| M55421 | 432978 |
| M55536 | 183299 |
| M55542 | 183001 |
| M55543 | 829176 |
| M57399 | 292072 |
| M57567 | 178986 |
| M58028 | 340071 |
| M58485 | 180154 |
| M58525 | 179954 |
| M58549 | 187592 |
| M58581 | 180988 |
| M59305 | 178651 |
| M59465 | 177865 |
| M59849 | 182591 |
| M60255 | 337759 |
| M60457 | 181249 |
| M60527 | 181509 |
| M60828 | 186738 |
| M60857 | 181334 |
| M61199 | 181122 |
| M61831 | 178276 |
| M61832 | 178278 |
| M61866 | 454818 |
| M61916 | 186836 |
| M62015 | 272265 |
| M62401 | 181291 |
| M62840 | 178068 |
| M63180 | 339679 |
| M63573 | 337998 |
| M63838 | 184568 |
| M64098 | 183891 |
| M64241 | 190813 |
| M64347 | 182564 |
| M64571 | 187382 |
| M64572 | 179912 |
| M65131 | 187451 |
| M65217 | 184404 |
| M68840 | 187352 |
| M68864 | 189396 |
| M69066 | 188625 |
| M69106 | 182999 |
| M69177 | 187358 |
| M69180 | 189029 |
| M69181 | 641957 |
| M69226 | 187354 |
| M69238 | 179003 |
| M73547 | 190161 |
| M73837 | 188685 |
| M74002 | 178996 |
| M74091 | 180627 |
| M74509 | 325464 |
| M74524 | 184043 |
| M74558 | 338087 |
| M74777 | 180082 |
| M76180 | 181520 |
| M76729 | 189519 |
| M76766 | 339489 |
| M77142 | 339700 |
| M77804 | 184656 |
| M77830 | 4689438 |
| M78906 | 273219 |
| M80783 | 179303 |
| M80902 | 178280 |
| M81757 | 337732 |
| M82882 | 180551 |
| M82919 | 182924 |
| M83205 | 184225 |
| M83653 | 179635 |
| M83738 | 190745 |
| M83822 | 1580780 |
| M83941 | 183931 |
| M84443 | 183265 |
| M84711 | 182774 |
| M85164 | 338034 |
| M85168 | 1785860 |
| M85357 | 274005 |
| M85323 | 274071 |
| M86752 | 184564 |
| M86917 | 189402 |
| M87284 | 338651 |
| M87339 | 1498255 |
| M88163 | 292495 |
| M88279 | 186389 |
| M90054 | 337579 |
| M90104 | 337925 |
| M90309 | 182643 |
| M90360 | 184434 |
| M90516 | 183081 |
| M92381 | 339660 |
| M92439 | 177109 |
| M92843 | 183442 |
| M93651 | 338038 |
| M94345 | 187455 |
| M94556 | 188855 |
| M94654 | 186379 |
| M94856 | 182353 |
| M95178 | 178051 |
| M95627 | 870802 |
| M95724 | 180246 |
| M96803 | 338442 |
| M97347 | 183440 |
| M97856 | 184432 |
| M97934 | 186566 |
| M97935 | 2281070 |
| M99701 | 521206 |
| M20046 | 1124713 |
| M20052 | 1124719 |
| M20305 | 1125260 |
| M20328 | 1125283 |
| N20338 | 1125293 |
| N20480 | 1125435 |
| N20482 | 1125437 |
| N20522 | 1125477 |
| N20593 | 1125548 |
| N20602 | 1125557 |
| N20820 | 1126001 |
| N20989 | 1126159 |
| N21081 | 1126251 |
| N21084 | 1126254 |
| N21170 | 1126340 |
| N21201 | 1126371 |
| N21233 | 1126403 |
| N21237 | 1126407 |
| N21334 | 1126504 |
| N21338 | 1126508 |
| N21576 | 1126746 |
| N21624 | 1126794 |
| N21633 | 1126803 |
| N21688 | 1126858 |
| N22007 | 1128141 |
| N22084 | 1128218 |
| N22210 | 1128344 |
| N22230 | 1128364 |
| N22272 | 1128406 |
| N22495 | 1128629 |
| N22687 | 1136837 |
| N22766 | 1136916 |
| N22836 | 1136986 |
| N23010 | 1137160 |
| N23139 | 1137289 |
| N23545 | 1137695 |
| N23605 | 1137755 |
| N23606 | 1137756 |
| N23652 | 1137802 |
| N23771 | 1137921 |
| N23865 | 1138015 |
| N23882 | 1138032 |
| N24042 | 1138192 |
| N24113 | 1138263 |
| N24155 | 1138305 |
| N24284 | 1138434 |
| N24437 | 1138587 |
| N24459 | 1138609 |
| N24538 | 1138688 |
| N24579 | 1138729 |
| N24785 | 1138935 |
| N24848 | 1138998 |
| N24869 | 1139019 |
| N24910 | 1139060 |
| N24973 | 1139123 |
| N25030 | 1139180 |
| N25122 | 1139272 |
| N25145 | 1139295 |
| N25262 | 1139412 |
| N25344 | 1139494 |
| N25552 | 1139900 |
| N25751 | 1140099 |
| N25883 | 1140231 |
| N25920 | 1140268 |
| N25936 | 1140284 |
| N26008 | 1140356 |
| N26026 | 1140374 |
| N26083 | 1140431 |
| N26163 | 1140511 |
| N26171 | 1140519 |
| N26359 | 1140707 |
| N26421 | 1140769 |
| N26515 | 1140863 |
| N26556 | 1140904 |
| N26559 | 1140907 |
| N26608 | 1140956 |
| N26724 | 1141072 |
| N26769 | 1141117 |
| N26993 | 1141341 |
| N27218 | 1141566 |
| N27272 | 1141620 |
| N27366 | 1141847 |
| N27610 | 1142091 |
| N27641 | 1142122 |
| N27899 | 1142380 |
| N27935 | 1142416 |
| N27992 | 1142473 |
| N28008 | 1142489 |
| N28268 | 1146504 |
| N28308 | 1146544 |
| N28331 | 1146567 |
| N28384 | 1146620 |
| N28426 | 1146662 |
| N28522 | 1146758 |
| N29457 | 1147977 |
| N29545 | 1148065 |
| N29624 | 1148144 |
| N29638 | 1148158 |
| N29796 | 1148316 |
| N29851 | 1148371 |
| N29901 | 1148421 |
| N29914 | 1148434 |
| N29918 | 1148438 |
| N29986 | 1148506 |
| N29992 | 1148512 |
| N30096 | 1148616 |
| N30098 | 1148618 |
| N30131 | 1148651 |
| N30185 | 1148705 |
| N30285 | 1148805 |
| N30328 | 1148848 |
| N30372 | 1148892 |
| N30486 | 1149006 |
| N30562 | 1149082 |
| N30570 | 1149090 |
| N30597 | 1149117 |
| N30655 | 1149175 |
| N30680 | 1149200 |
| N30699 | 1149219 |
| N30704 | 1149224 |
| N30751 | 1149271 |
| N31301 | 1151700 |
| N31312 | 1151711 |
| N31529 | 1151928 |
| N31564 | 1151963 |
| N31605 | 1152004 |
| N31641 | 1152040 |
| N31801 | 1152200 |
| N31808 | 1152207 |
| N31825 | 1152224 |
| N31935 | 1152334 |
| N31952 | 1152351 |
| N32044 | 1152443 |
| N32071 | 1152470 |
| N32072 | 1152471 |
| N32095 | 1152494 |
| N32192 | 1152591 |
| N32226 | 1152625 |
| N32485 | 1152884 |
| N32514 | 1152913 |
| N32542 | 1152941 |
| N32587 | 1152986 |
| N32594 | 1152993 |
| N32737 | 1153136 |
| N32768 | 1153167 |
| N32904 | 1153303 |
| N33041 | 1153440 |
| N33228 | 1153627 |
| N33263 | 1153662 |
| N33264 | 1153663 |
| N33274 | 1153673 |
| N33323 | 1153722 |
| N33331 | 1153730 |
| N33366 | 1153765 |
| N33443 | 1153842 |
| N33550 | 1153949 |
| N33567 | 1153966 |
| N33778 | 1154183 |
| N33927 | 1154327 |
| N33965 | 1154365 |
| N34042 | 1154442 |
| N34117 | 1154517 |
| N34111 | 1154541 |
| N34186 | 1154586 |
| N34316 | 1155458 |
| N34395 | 1155537 |
| N34436 | 1155578 |
| N34482 | 1155624 |
| N34611 | 1155753 |
| N34824 | 1155966 |
| N34892 | 1156034 |
| N34895 | 1156037 |
| N34924 | 1156066 |
| N34943 | 1156085 |
| N34945 | 1156087 |
| N35020 | 1156162 |
| N35067 | 1156209 |
| N35369 | 1156511 |
| N35489 | 1156631 |
| N35555 | 1156697 |
| N35579 | 1156721 |
| N35603 | 1156745 |
| N35772 | 1156914 |
| N35863 | 1157005 |
| N35888 | 1157030 |
| N35907 | 1157049 |
| N35922 | 1157064 |
| N36008 | 1157150 |
| N36090 | 1157232 |
| N36233 | 1157375 |
| N36327 | 1157469 |
| N36389 | 1157531 |
| N36402 | 1157544 |
| N36794 | 1157936 |
| N36967 | 1158109 |
| N36968 | 1158110 |
| N38860 | 1162067 |
| N38992 | 1162199 |
| N39233 | 1162440 |
| N39338 | 1162545 |
| N39426 | 1162633 |
| N39590 | 1162797 |
| N39611 | 1162818 |
| N39674 | 1162881 |
| N39836 | 1163381 |
| N40017 | 1163562 |
| N40101 | 1163646 |
| N40202 | 1163747 |
| N40262 | 1163807 |
| N40375 | 1163972 |
| N40582 | 1164179 |
| N40598 | 1164195 |
| N40717 | 1164314 |
| N40743 | 1164340 |
| N40852 | 1164449 |
| N40939 | 1164537 |
| N40951 | 1164549 |
| N40952 | 1164550 |
| N40976 | 1164574 |
| N41386 | 1165417 |
| N41418 | 1165449 |
| N41773 | 1165804 |
| N41826 | 1165857 |
| N41827 | 1165858 |
| N42196 | 1166227 |
| N42772 | 1167202 |
| N42929 | 1166673 |
| N42970 | 1166714 |
| N42986 | 1166730 |
| N43976 | 1182504 |
| N44001 | 1182529 |
| N44337 | 1182855 |
| N44743 | 1185909 |
| N45083 | 1186249 |
| N45167 | 1186333 |
| N45223 | 1186389 |
| N45236 | 1186402 |
| N45282 | 1186448 |
| N45398 | 1186564 |
| N45940 | 1187106 |
| N45983 | 1187149 |
| N46575 | 1187741 |
| N46759 | 1187925 |
| N46845 | 1188011 |
| N46947 | 1188113 |
| N47008 | 1188174 |
| N47012 | 1188178 |
| N47044 | 1188210 |
| N47075 | 1188241 |
| N47113 | 1188279 |
| N47208 | 1188374 |
| N47214 | 1188380 |
| N47240 | 1188406 |
| N47292 | 1188458 |
| N47308 | 1188474 |
| N47316 | 1188482 |
| N47360 | 1188526 |
| N47425 | 1188591 |
| N47443 | 1188609 |
| N47445 | 1188611 |
| N47604 | 1188770 |
| N47691 | 1188857 |
| N47717 | 1188883 |
| N47738 | 1188904 |
| N47829 | 1188995 |
| N47886 | 1189052 |
| N47902 | 1189068 |
| N47941 | 1189107 |
| N47952 | 1189118 |
| N47954 | 1189120 |
| N47961 | 1189127 |
| N48050 | 1189216 |
| N48075 | 1189241 |
| N48078 | 1189244 |
| N48259 | 1189425 |
| N48261 | 1189427 |
| N48271 | 1189437 |
| N48302 | 1189468 |
| N48325 | 1189491 |
| N48356 | 1189522 |
| N48580 | 1189746 |
| N48590 | 1189756 |
| N48701 | 1189867 |
| N48707 | 1189873 |
| N48804 | 1189970 |
| N48809 | 1189975 |
| N48901 | 1190067 |
| N48913 | 1190079 |
| N49079 | 1190245 |
| N49098 | 1190264 |
| N49107 | 1190273 |
| N49109 | 1190275 |
| N49181 | 1190347 |
| N49186 | 1190352 |
| N49261 | 1190427 |
| N49346 | 1190512 |
| N49389 | 1190555 |
| N49405 | 1190571 |
| N49471 | 1190637 |
| N49534 | 1190700 |
| N49548 | 1190714 |
| N49574 | 1190740 |
| N49577 | 1190743 |
| N49589 | 1190755 |
| N49605 | 1190771 |
| N49619 | 1190785 |
| N49629 | 1190795 |
| N49725 | 1190891 |
| N49899 | 1191065 |
| N50079 | 1191245 |
| N50432 | 1191598 |
| N50563 | 1191729 |
| N50647 | 1191813 |
| N50729 | 1191895 |
| N50733 | 1191899 |
| N50745 | 1191911 |
| N50787 | 1191953 |
| N50797 | 1191963 |
| N50806 | 1191972 |
| N50827 | 1191993 |
| N50828 | 1191994 |
| N50843 | 1192009 |
| N50853 | 1192019 |
| N50859 | 1192025 |
| N50864 | 1192030 |
| N50880 | 1192046 |
| N50904 | 1192070 |
| N50912 | 1192078 |
| N50935 | 1192101 |
| N50962 | 1192128 |
| N50983 | 1192149 |
| N51002 | 1192168 |
| N51069 | 1192235 |
| N51107 | 1192273 |
| N51226 | 1192392 |
| N51280 | 1192446 |
| N51290 | 1192456 |
| N51297 | 1192463 |
| N51335 | 1192501 |
| N51343 | 1192509 |
| N51357 | 1192523 |
| N51362 | 1192528 |
| N51367 | 1192533 |
| N51441 | 1192607 |
| N51498 | 1192664 |
| N51563 | 1192729 |
| N51574 | 1192740 |
| N51583 | 1192749 |
| N51709 | 1192875 |
| N51740 | 1192906 |
| N51806 | 1192972 |
| N51883 | 1193049 |
| N51987 | 1193153 |
| N52151 | 1193412 |
| N52158 | 1193419 |
| N52178 | 1193312 |
| N52193 | 1193327 |
| N52271 | 1193437 |
| N52350 | 1193516 |
| N52432 | 1193598 |
| N52450 | 1193616 |
| N52554 | 1193720 |
| N52589 | 1193755 |
| N52675 | 1193841 |
| N52767 | 1193933 |
| N52837 | 1194003 |
| N52875 | 1194041 |
| N52876 | 1194042 |
| N52911 | 1194077 |
| N52973 | 1194139 |
| N53031 | 1194197 |
| N53033 | 1194199 |
| N53133 | 1194299 |
| N53214 | 1194380 |
| N53378 | 1194544 |
| N53380 | 1194546 |
| N53421 | 1194587 |
| N53447 | 1194613 |
| N53456 | 1194622 |
| N53492 | 1194658 |
| N53534 | 1194700 |
| N53940 | 1195106 |
| N53959 | 1195125 |
| N54036 | 1195202 |
| N54254 | 1195420 |
| N54512 | 1195832 |
| N54551 | 1195871 |
| N54596 | 1195916 |
| N54728 | 1196048 |
| N54763 | 1196083 |
| N54788 | 1196108 |
| N54973 | 1196293 |
| N55167 | 1198046 |
| N55266 | 1198145 |
| N55339 | 1198218 |
| N55430 | 1198309 |
| N55459 | 1198338 |
| N56237 | 1199085 |
| N56690 | 1200580 |
| N56875 | 1200765 |
| N56982 | 1200872 |
| N57002 | 1200892 |
| N57475 | 1201365 |
| N57483 | 1201373 |
| N57526 | 1201416 |
| N57722 | 1201612 |
| N57858 | 1201748 |
| N57872 | 1201762 |
| N58052 | 1201942 |
| N58107 | 1201997 |
| N58136 | 1202026 |
| N58148 | 1202038 |
| N58170 | 1202060 |
| N58276 | 1202166 |
| N58417 | 1202307 |
| N58558 | 1202448 |
| N58818 | 1202708 |
| N59078 | 1202968 |
| N59119 | 1203009 |
| N59136 | 1203026 |
| N59158 | 1203048 |
| N59206 | 1203096 |
| N59278 | 1203168 |
| N59295 | 1203185 |
| N59336 | 1203226 |
| N59532 | 1203422 |
| N59626 | 1203516 |
| N59808 | 1203698 |
| N59816 | 1203706 |
| N59826 | 1203716 |
| N59866 | 1203756 |
| N59870 | 1203760 |
| N60172 | 1206323 |
| N62079 | 1210008 |
| N62096 | 1209909 |
| N62128 | 1209941 |
| N62132 | 1209945 |
| N62179 | 1209992 |
| N62244 | 1210073 |
| N62245 | 1210074 |
| N62251 | 1210080 |
| N62271 | 1210100 |
| N62293 | 1210122 |
| N62301 | 1210130 |
| N62332 | 1210161 |
| N62372 | 1210201 |
| N62376 | 1210205 |
| N62394 | 1210223 |
| N62403 | 1210232 |
| N62448 | 1210277 |
| N62498 | 1210327 |
| N62696 | 1210525 |
| N62726 | 1210555 |
| N62745 | 1210574 |
| N62763 | 1210592 |
| N62766 | 1210595 |
| N62780 | 1210609 |
| N62799 | 1210628 |
| N62817 | 1210646 |
| N62866 | 1210695 |
| N62924 | 1210753 |
| N62936 | 1210765 |
| N62948 | 1210777 |
| N63057 | 1210886 |
| N63153 | 1210982 |
| N63172 | 1211001 |
| N63286 | 1211115 |
| N63375 | 1211204 |
| N63387 | 1211216 |
| N63436 | 1211265 |
| N63543 | 1211372 |
| N63567 | 1211396 |
| N63609 | 1211438 |
| N63646 | 1211475 |
| N63727 | 1211556 |
| N63744 | 1211573 |
| N63768 | 1211597 |
| N63784 | 1211613 |
| N63807 | 1211636 |
| N63848 | 1211677 |
| N63988 | 1211817 |
| N63991 | 1211820 |
| N64379 | 1212208 |
| N64391 | 1212220 |
| N64508 | 1212337 |
| N64576 | 1212405 |
| N64597 | 1212426 |
| N64603 | 1212432 |
| N64617 | 1212446 |
| N64706 | 1212535 |
| N64762 | 1212591 |
| N64774 | 1212603 |
| N64796 | 1212625 |
| N64814 | 1212643 |
| N64840 | 1212669 |
| N65950 | 1218075 |
| N66085 | 1218210 |
| N66132 | 1218257 |
| N66135 | 1218260 |
| N66139 | 1218264 |
| N66158 | 1218283 |
| N66178 | 1218303 |
| N66203 | 1218328 |
| N66278 | 1218403 |
| N66346 | 1218471 |
| N66348 | 1218473 |
| N66393 | 1218518 |
| N66454 | 1218579 |
| N66572 | 1218697 |
| N66580 | 1218705 |
| N66724 | 1218849 |
| N66734 | 1218859 |
| N66737 | 1218862 |
| N66900 | 1219025 |
| N66925 | 1219050 |
| N66957 | 1219082 |
| N67039 | 1219164 |
| N67109 | 1219234 |
| N67168 | 1219293 |
| N67274 | 1219399 |
| N67570 | 1219695 |
| N67571 | 1219696 |
| N67619 | 1219744 |
| N67762 | 1219887 |
| N67766 | 1219891 |
| N67832 | 1219957 |
| N67861 | 1219986 |
| N67891 | 1220016 |
| N68399 | 1224560 |
| N68465 | 1224626 |
| N68576 | 1224737 |
| N68686 | 1224847 |
| N68719 | 1224880 |
| N68928 | 1225089 |
| N68998 | 1225159 |
| N69068 | 1225229 |
| N69283 | 1225444 |
| N69425 | 1225586 |
| N69491 | 1225652 |
| N69499 | 1225660 |
| N69528 | 1225689 |
| N69653 | 1225814 |
| N69675 | 1225836 |
| N69876 | 1226456 |
| N69989 | 1226569 |
| N70038 | 1226618 |
| N70592 | 1227172 |
| N70654 | 1227234 |
| N70714 | 1227294 |
| N70791 | 1227371 |
| N70794 | 1227374 |
| N71080 | 1227660 |
| N71769 | 1228481 |
| N71861 | 1228573 |
| N71982 | 1228694 |
| N72115 | 1229219 |
| N72137 | 1229241 |
| N72150 | 1229254 |
| N72196 | 1229300 |
| N72215 | 1229319 |
| N72263 | 1229367 |
| N72274 | 1229378 |
| N72458 | 1229562 |
| N72648 | 1229752 |
| N72697 | 1229801 |
| N72922 | 1230026 |
| N73033 | 1230137 |
| N73083 | 1230187 |
| N73091 | 1230195 |
| N73101 | 1230205 |
| N73252 | 1230356 |
| N73301 | 1230405 |
| N73480 | 1230765 |
| N73563 | 1230848 |
| N73584 | 1230869 |
| N73958 | 1231243 |
| N74131 | 1231416 |
| N74267 | 1231552 |
| N74392 | 1231677 |
| N74617 | 1231902 |
| N74623 | 1231908 |
| N74889 | 1237568 |
| N74956 | 1237502 |
| N74995 | 1237541 |
| N75055 | 1237633 |
| N75318 | 1237896 |
| N75356 | 1237934 |
| N75394 | 1237972 |
| N75468 | 1238046 |
| N75473 | 1238051 |
| N75893 | 1238471 |
| N75947 | 1238525 |
| N75979 | 1238557 |
| N76088 | 1238666 |
| N76101 | 1238679 |
| N76201 | 1238779 |
| N76215 | 1238793 |
| N76672 | 1239250 |
| N77134 | 1239712 |
| N77263 | 1239841 |
| N77277 | 1239855 |
| N77514 | 1240215 |
| N77779 | 1240480 |
| N77828 | 1240529 |
| N78083 | 1240784 |
| N78092 | 1240793 |
| N78279 | 1240980 |
| N78903 | 1241604 |
| N78927 | 1241628 |
| N79180 | 1241881 |
| N79230 | 1241931 |
| N79353 | 1242054 |
| N79427 | 1242128 |
| N79712 | 1242413 |
| N79738 | 1242439 |
| N79745 | 1242446 |
| N79747 | 1242448 |
| N79989 | 1242690 |
| N80129 | 1242830 |
| N80764 | 1243465 |
| N81049 | 1243750 |
| N81158 | 1243859 |
| N81205 | 1243906 |
| N86292 | 1439296 |
| N86927 | 1440129 |
| N89671 | 1442998 |
| N89676 | 1443003 |
| N89783 | 1443110 |
| N89861 | 1443188 |
| N89973 | 1443300 |
| N90051 | 1443378 |
| N90514 | 1443841 |
| N90523 | 1443850 |
| N90583 | 1443910 |
| N90779 | 1444106 |
| N90806 | 1444133 |
| N90882 | 1444209 |
| N90917 | 1444244 |
| N91003 | 1444330 |
| N91096 | 1444423 |
| N91117 | 1444444 |
| N91175 | 1444502 |
| N91258 | 1444585 |
| N91259 | 1444586 |
| N91382 | 1444709 |
| N91385 | 1444712 |
| N91566 | 1444893 |
| N91744 | 1264053 |
| N91767 | 1264076 |
| N91797 | 1264106 |
| N91811 | 1264120 |
| N91825 | 1264134 |
| N91887 | 1264196 |
| N91914 | 1264223 |
| N91921 | 1264230 |
| N92160 | 1264469 |
| N92310 | 1264619 |
| N92478 | 1264787 |
| N92483 | 1264792 |
| N92498 | 1264807 |
| N92646 | 1264955 |
| N92699 | 1265008 |
| N92755 | 1265064 |
| N92764 | 1265073 |
| N92842 | 1265151 |
| N92895 | 1265204 |
| N92901 | 1265210 |
| N93236 | 1265545 |
| N93428 | 1265737 |
| N93438 | 1265747 |
| N93470 | 1265779 |
| N93715 | 1266024 |
| N93721 | 1266030 |
| N93790 | 1266099 |
| N93924 | 1266233 |
| N94098 | 1266407 |
| N94111 | 1266420 |
| N94259 | 1266568 |
| N94351 | 1266660 |
| N94487 | 1266796 |
| N94829 | 1267170 |
| N94855 | 1267125 |
| N95007 | 1267289 |
| N95187 | 1267478 |
| N95226 | 1267507 |
| N95435 | 1267706 |
| N95476 | 1267767 |
| N95495 | 1267834 |
| N98283 | 1269706 |
| N98328 | 1269973 |
| N98569 | 1269992 |
| N98593 | 1270208 |
| N98672 | 1270094 |
| N98749 | 1270172 |
| N99088 | 1270541 |
| N99553 | 1270966 |
| N99659 | 1271101 |
| N99698 | 1271140 |
| N99711 | 1271153 |
| N99839 | 1271382 |
| R00128 | 749864 |
| R00275 | 750011 |
| R00276 | 750012 |
| R00283 | 750019 |
| R00284 | 750020 |
| R00479 | 750215 |
| R00480 | 750216 |
| R00707 | 750443 |
| R00809 | 750545 |
| R00884 | 750620 |
| R01094 | 750830 |
| R01A39 | 750875 |
| R01245 | 750981 |
| R01304 | 751040 |
| R01348 | 751084 |
| ROlSiS | 751251 |
| R01637 | 751373 |
| R01739 | 751475 |
| R01937 | 751673 |
| R02439 | 752175 |
| R02586 | 752322 |
| R05416 | 756036 |
| R05660 | 756280 |
| R05693 | 756313 |
| R05810 | 756430 |
| R05886 | 756506 |
| R06119 | 756739 |
| R06307 | 756927 |
| R06362 | 756982 |
| R06372 | 756992 |
| R06479 | 757099 |
| R06706 | 757326 |
| R06716 | 757336 |
| R06862 | 757482 |
| R06944 | 758867 |
| R07012 | 758935 |
| R07115 | 759038 |
| R07184 | 759107 |
| R07196 | 759119 |
| R07506 | 759429 |
| R07891 | 759814 |
| R07948 | 759871 |
| R08121 | 760044 |
| R08266 | 760189 |
| R08292 | 760215 |
| R08658 | 760581 |
| R08871 | 760794 |
| R08932 | 760855 |
| R08935 | 760858 |
| R08938 | 760861 |
| R09179 | 761102 |
| R09504 | 761427 |
| R09729 | 761652 |
| R09747 | 761670 |
| R09980 | 761936 |
| R10011 | 761967 |
| R10015 | 761971 |
| R10140 | 762096 |
| R10154 | 762110 |
| R10284 | 762240 |
| R10301 | 762257 |
| R10378 | 762334 |
| R10438 | 762394 |
| R10553 | 762509 |
| R10935 | 763670 |
| R10947 | 763682 |
| R10948 | 763683 |
| R11114 | 763849 |
| R11236 | 763971 |
| R11516 | 764245 |
| R11581 | 764316 |
| R11587 | 764322 |
| R11605 | 764340 |
| R11877 | 764612 |
| R11885 | 764620 |
| R11888 | 764623 |
| R12113 | 764848 |
| R12183 | 764918 |
| R12356 | 765432 |
| R12385 | 765461 |
| R12449 | 765525 |
| R12473 | 765549 |
| R12804 | 765880 |
| R12808 | 765884 |
| R13434 | 766510 |
| R13557 | 766633 |
| R13761 | 766837 |
| R13850 | 766926 |
| R13878 | 766954 |
| R14008 | 767084 |
| R14230 | 767306 |
| R14619 | 768892 |
| R14631 | 763904 |
| R14684 | 768957 |
| R14689 | 768962 |
| R14699 | 768972 |
| R15409 | 768157 |
| R15740 | 768024 |
| R15779 | 768063 |
| R15784 | 768199 |
| R15813 | 768228 |
| R15870 | 768285 |
| R15880 | 768295 |
| R15891 | 768306 |
| R16146 | 768074 |
| R16153 | 768081 |
| R16241 | 768489 |
| R16245 | 768493 |
| R16259 | 768507 |
| R16295 | 769905 |
| R16408 | 770018 |
| R16541 | 770151 |
| R16596 | 770206 |
| R16676 | 770286 |
| R16957 | 770567 |
| R17092 | 770702 |
| R17096 | 770706 |
| R17110 | 770720 |
| R17320 | 770930 |
| R17476 | 771086 |
| R17747 | 771357 |
| R17962 | 771572 |
| R18215 | 771825 |
| R18258 | 771868 |
| R18274 | 771884 |
| R18412 | 772022 |
| R18975 | 772585 |
| R19183 | 772793 |
| R19453 | 773063 |
| R19478 | 773088 |
| R19498 | 773108 |
| R19761 | 774395 |
| R19869 | 774503 |
| R19878 | 774512 |
| R19977 | 774611 |
| R20063 | 774697 |
| R20331 | 774965 |
| R20424 | 775058 |
| R20547 | 820487 |
| R20616 | 775397 |
| R20628 | 775409 |
| R20638 | 775419 |
| R20639 | 775420 |
| R20640 | 775421 |
| R20651 | 775432 |
| R20669 | 775450 |
| R20813 | 775594 |
| R22003 | 776784 |
| R22024 | 776805 |
| R22088 | 776869 |
| R22136 | 776917 |
| R22156 | 776937 |
| R22206 | 776987 |
| R22308 | 777089 |
| R22579 | 777360 |
| R22748 | 777573 |
| R23055 | 777943 |
| R23241 | 778129 |
| R23246 | 778134 |
| R23270 | 778158 |
| R23609 | 778497 |
| R23727 | 778615 |
| R23735 | 778623 |
| R23790 | 778678 |
| R23858 | 778746 |
| R24400 | 779288 |
| R24451 | 779339 |
| R24506 | 779394 |
| R24803 | 779691 |
| R24834 | 779722 |
| R25166 | 780054 |
| R25377 | 781512 |
| R25403 | 781538 |
| R25464 | 781599 |
| R25577 | 781712 |
| R25614 | 781749 |
| R25807 | 781942 |
| R25823 | 781958 |
| R26186 | 782321 |
| R26206 | 782341 |
| R26444 | 782579 |
| R26456 | 782591 |
| R26785 | 782920 |
| R26859 | 782994 |
| R26960 | 783095 |
| R27004 | 783139 |
| R27193 | 783328 |
| R27450 | 783585 |
| R27767 | 783902 |
| R28254 | 784389 |
| R28287 | 784422 |
| R28397 | 784532 |
| R28584 | 784719 |
| R28603 | 784738 |
| R28660 | 784795 |
| R31114 | 786957 |
| R31413 | 787256 |
| R31701 | 787544 |
| R31789 | 787632 |
| R32406 | 788249 |
| R32613 | 788456 |
| R32952 | 788795 |
| R33103 | 788961 |
| R33303 | 789161 |
| R33351 | 789209 |
| R33353 | 789211 |
| R33609 | 789467 |
| R33762 | 789620 |
| R33779 | 789637 |
| R33917 | 789775 |
| R34225 | 790083 |
| R34273 | 790131 |
| R34284 | 790142 |
| R34331 | 790189 |
| R34443 | 791344 |
| R34553 | 791454 |
| R34894 | 791795 |
| R35051 | 791952 |
| R35230 | 792131 |
| R35283 | 792184 |
| R35529 | 792430 |
| R35665 | 792566 |
| R35797 | 792698 |
| R35943 | 792844 |
| R36409 | 793310 |
| R36465 | 793366 |
| R36477 | 793378 |
| R36523 | 793424 |
| R36587 | 793488 |
| R36969 | 794425 |
| R36989 | 794445 |
| R37079 | 794535 |
| R37224 | 794680 |
| R37289 | 794745 |
| R37357 | 794813 |
| R37395 | 794851 |
| R37519 | 794975 |
| R37738 | 795194 |
| R37780 | 795236 |
| R38017 | 795473 |
| R38169 | 795625 |
| R38179 | 795635 |
| R38261 | 795717 |
| R38364 | 795820 |
| R38369 | 795825 |
| R38412 | 795868 |
| R38539 | 795995 |
| R38613 | 796069 |
| R38640 | 796096 |
| R38645 | 796101 |
| R38678 | 796134 |
| R38809 | 796265 |
| R38891 | 796347 |
| R38894 | 796350 |
| R38935 | 796391 |
| P28943 | 796399 |
| R38944 | 796400 |
| R38952 | 796408 |
| R39014 | 796470 |
| R39066 | 796522 |
| R39069 | 796525 |
| R39111 | 796567 |
| R39191 | 796647 |
| R39325 | 796781 |
| R39364 | 796820 |
| R39446 | 796902 |
| R39716 | 797172 |
| R39765 | 797221 |
| R39862 | 797478 |
| R39891 | 797507 |
| R39901 | 797517 |
| R39951 | 797567 |
| R40018 | 820767 |
| R40037 | 820786 |
| R40057 | 822754 |
| R40105 | 820806 |
| R40176 | 822802 |
| R40231 | 820875 |
| R40244 | 821032 |
| R40373 | 821116 |
| R40434 | 820883 |
| R40481 | 822861 |
| R40663 | 821001 |
| R40780 | 823031 |
| R40970 | 821229 |
| R41329 | 816637 |
| R41357 | 816664 |
| R41389 | 816695 |
| R41685 | 816978 |
| R41782 | 817487 |
| R41804 | 817508 |
| R41943 | 817640 |
| R41972 | 817667 |
| R41994 | 817689 |
| R42061 | 817007 |
| R42112 | 800336 |
| R42331 | 825268 |
| R42490 | 817254 |
| R42520 | 817283 |
| R42533 | 817295 |
| R42569 | 817331 |
| R42583 | 817345 |
| R42600 | 817361 |
| R42685 | 819630 |
| R42736 | 819675 |
| R42763 | 800987 |
| R42780 | 819699 |
| R42823 | 819734 |
| R42831 | 819742 |
| R42946 | 819851 |
| R42994 | 820056 |
| R43017 | 820079 |
| R43053 | 820114 |
| R43289 | 825415 |
| R43205 | 825430 |
| R43271 | 821378 |
| R43308 | 821415 |
| R43319 | 821426 |
| R43328 | 819855 |
| R43432 | 819950 |
| R43452 | 819970 |
| R43485 | 820003 |
| R43535 | 821464 |
| R43553 | 821482 |
| R43566 | 821493 |
| R43699 | 821612 |
| R43798 | 823647 |
| R43805 | 823652 |
| R43910 | 821788 |
| R43915 | 821793 |
| R44048 | 821916 |
| R44078 | 821946 |
| R44132 | 821997 |
| R44164 | 822028 |
| R44201 | 822064 |
| R44292 | 821263 |
| R44346 | 820642 |
| R44404 | 823302 |
| R44447 | 823344 |
| R44530 | 823920 |
| R44538 | 823927 |
| R44544 | 823933 |
| R44617 | 824005 |
| R44664 | 824046 |
| R44734 | 824112 |
| R44741 | 824119 |
| R44754 | 824132 |
| R44770 | 824147 |
| R44776 | 824153 |
| R44816 | 824193 |
| R44837 | 824212 |
| R44840 | 824215 |
| R44936 | 823203 |
| R44985 | 824339 |
| R45114 | 823468 |
| R45118 | 823472 |
| R45144 | 823498 |
| R45157 | 823511 |
| R45192 | 823546 |
| R45264 | 821667 |
| R45292 | 822151 |
| R45402 | 823670 |
| R45470 | 820725 |
| R45517 | 823731 |
| R45632 | 823844 |
| R45672 | 822118 |
| R45686 | 822132 |
| R45952 | 824285 |
| R45964 | 823208 |
| R46000 | 823239 |
| R46003 | 823242 |
| R46295 | 805692 |
| R46700 | 822667 |
| R46794 | 822611 |
| R46816 | 822633 |
| R46837 | 822654 |
| R47893 | 809919 |
| R47979 | 810005 |
| R48046 | 810072 |
| R48320 | 810346 |
| R48833 | 810859 |
| R48890 | 810916 |
| R48977 | 817743 |
| R49052 | 817814 |
| R49124 | 820193 |
| R49126 | 820195 |
| R49436 | 820334 |
| R49439 | 820337 |
| R49462 | 820360 |
| R49555 | 820399 |
| R49592 | 825123 |
| R49644 | 825174 |
| R49695 | 825225 |
| R49731 | 820445 |
| R49734 | 811636 |
| R50104 | 812006 |
| R50277 | 812179 |
| R50467 | 812369 |
| R50752 | 812654 |
| R50775 | 812677 |
| R51064 | 812966 |
| R51080 | 812982 |
| R51100 | 813002 |
| R51236 | 813138 |
| R51261 | 813163 |
| R51273 | 813175 |
| R51382 | 813284 |
| R51416 | 813318 |
| R51506 | 813408 |
| R51535 | 813437 |
| R51540 | 813442 |
| R51617 | 813519 |
| R51737 | 813639 |
| R51865 | 813767 |
| R51872 | 813774 |
| R51912 | 813814 |
| R52015 | 813917 |
| R52082 | 813984 |
| R52085 | 813987 |
| R52088 | 813990 |
| R52089 | 813991 |
| R52387 | 814289 |
| R52526 | 814428 |
| R52530 | 814432 |
| R52543 | 814445 |
| R52624 | 814526 |
| R52640 | 814542 |
| R52729 | 814631 |
| R52786 | 814688 |
| R52789 | 814691 |
| R52794 | 814696 |
| R52796 | 814698 |
| R52901 | 814803 |
| R53072 | 814974 |
| R53294 | 815196 |
| R53311 | 815213 |
| R53342 | 815244 |
| R53348 | 815250 |
| R53428 | 815330 |
| R53446 | 815348 |
| R53455 | 815357 |
| R53558 | 815460 |
| R53757 | 815659 |
| R53758 | 815660 |
| R53943 | 815845 |
| R53951 | 815853 |
| R53963 | 815865 |
| R54036 | 815938 |
| R54105 | 816007 |
| R54109 | 816011 |
| R54179 | 816081 |
| R54212 | 816114 |
| R54273 | 816175 |
| R54492 | 816394 |
| R54590 | 816492 |
| R54594 | 816496 |
| R54846 | 818968 |
| R54850 | 818972 |
| R54855 | 818977 |
| R55220 | 824515 |
| R55261 | 824556 |
| R55535 | 824830 |
| R55543 | 824838 |
| R55600 | 824895 |
| R55619 | 824914 |
| R55750 | 825825 |
| R55882 | 825988 |
| R55965 | 826071 |
| R56046 | 826152 |
| R56055 | 826161 |
| R56100 | 826206 |
| R56106 | 826212 |
| R56123 | 826229 |
| R56149 | 826255 |
| R56382 | 826488 |
| R56604 | 826710 |
| R56613 | 826719 |
| R56642 | 826748 |
| R56771 | 826877 |
| R56840 | 826946 |
| R56854 | 826960 |
| R56870 | 826976 |
| R56877 | 826983 |
| R56885 | 826991 |
| R56898 | 827004 |
| R56906 | 827012 |
| R58953 | 829648 |
| R59031 | 829726 |
| R59068 | 829763 |
| R59087 | 829782 |
| R59147 | 829842 |
| R59197 | 829892 |
| R59304 | 829999 |
| R59369 | 830064 |
| R59473 | 830168 |
| R59556 | 830251 |
| R59618 | 830313 |
| R59621 | 830316 |
| R59722 | 830417 |
| R59789 | 830484 |
| R59846 | 830541 |
| R59936 | 830631 |
| R59990 | 830685 |
| R60014 | 830709 |
| R60019 | 830714 |
| R60020 | 830715 |
| R60223 | 830918 |
| R60328 | 831023 |
| R60395 | 831090 |
| R60705 | 831400 |
| R60713 | 831408 |
| R60717 | 831412 |
| R60722 | 831417 |
| R60731 | 831426 |
| R60847 | 831542 |
| R60946 | 831641 |
| R60949 | 831644 |
| R60981 | 831676 |
| R60995 | 831690 |
| R61073 | 831768 |
| R61126 | 831821 |
| R61189 | 831884 |
| R61196 | 831891 |
| R61372 | 832067 |
| R61374 | 832069 |
| R61395 | 832090 |
| R61436 | 832131 |
| R61556 | 832251 |
| R61601 | 832296 |
| R61734 | 832429 |
| R61780 | 832475 |
| R61796 | 832491 |
| R61847 | 832542 |
| R61866 | 832561 |
| R61871 | 832566 |
| R61883 | 832578 |
| R62289 | 834168 |
| R62352 | 834231 |
| R62470 | 834349 |
| R62742 | 834621 |
| R62780 | 834659 |
| R62926 | 834805 |
| R63022 | 834901 |
| R63085 | 834964 |
| R63106 | 834985 |
| R63129 | 835008 |
| R63251 | 835130 |
| R63253 | 835132 |
| R63646 | 835525 |
| R63823 | 835702 |
| R64194 | 836073 |
| R64231 | 836110 |
| R64454 | 836333 |
| R64693 | 836572 |
| R65963 | 838601 |
| R66006 | 838644 |
| R66082 | 838720 |
| R66101 | 838739 |
| R66162 | 838800 |
| R66438 | 839076 |
| R66923 | 839561 |
| R67081 | 839719 |
| R67147 | 839785 |
| R67218 | 839856 |
| R67259 | 839897 |
| R67466 | 840104 |
| R68272 | 841789 |
| R68409 | 841926 |
| R68581 | 842098 |
| R68909 | 842426 |
| R69584 | 843101 |
| R69724 | 843241 |
| R69818 | 843335 |
| R69934 | 843451 |
| R69940 | 843457 |
| R69943 | 843460 |
| R70116 | 843633 |
| R70193 | 843710 |
| R70270 | 843787 |
| R70570 | 844087 |
| R70598 | 844115 |
| R70639 | 844156 |
| R70649 | 844166 |
| R70675 | 844192 |
| R70684 | 844201 |
| R70685 | 844202 |
| R70995 | 844512 |
| R71124 | 844641 |
| R71335 | 844852 |
| R71393 | 844910 |
| R71131 | 844948 |
| R71619 | 845136 |
| R71689 | 845721 |
| R72215 | 846247 |
| R72403 | 846435 |
| R72434 | 846466 |
| R72518 | 846550 |
| R72618 | 846650 |
| R72738 | 846770 |
| R72768 | 846800 |
| R72977 | 847009 |
| R73088 | 847120 |
| R73525 | 847557 |
| R73545 | 847577 |
| R74152 | 848522 |
| R74169 | 848539 |
| R74203 | 848573 |
| R74321 | 848691 |
| R74478 | 848848 |
| R74597 | 848967 |
| R75754 | 850436 |
| R76229 | 850911 |
| R76235 | 850917 |
| R76244 | 850926 |
| R76247 | 850929 |
| R76281 | 850963 |
| R76505 | 851154 |
| R76782 | 851414 |
| R77185 | 851817 |
| R77186 | 851818 |
| R77512 | 852622 |
| R77718 | 852828 |
| R77854 | 852964 |
| R77919 | 853029 |
| R77948 | 853058 |
| R78386 | 853496 |
| R78559 | 854840 |
| R78584 | 854865 |
| R78607 | 854888 |
| R78725 | 855006 |
| R80217 | 856498 |
| R80235 | 856516 |
| R80279 | 856560 |
| R80387 | 856668 |
| R80790 | 857071 |
| R80852 | 857133 |
| R81053 | 857334 |
| R81486 | 858089 |
| R81831 | 858434 |
| R82041 | 858644 |
| R82299 | 861690 |
| R82317 | 861708 |
| R82429 | 861820 |
| R82456 | 861847 |
| R82644 | 862035 |
| R82733 | 862124 |
| R83407 | 928284 |
| R83716 | 928593 |
| R83896 | 928773 |
| R84308 | 942714 |
| R84398 | 942804 |
| R85291 | 943697 |
| R86242 | 944648 |
| R86265 | 944671 |
| R86847 | 945484 |
| R86970 | 945711 |
| R87758 | 946571 |
| R87877 | 946690 |
| R88246 | 947059 |
| R88267 | 947080 |
| R88370 | 950265 |
| R88764 | 953591 |
| R88904 | 953731 |
| R88915 | 953742 |
| R88918 | 953745 |
| R89082 | 953909 |
| R89287 | 954114 |
| R89317 | 954144 |
| R89358 | 954185 |
| R89363 | 954190 |
| R89403 | 954230 |
| R89490 | 954317 |
| R89611 | 954438 |
| R89700 | 954527 |
| R89765 | 954592 |
| R89846 | 954673 |
| R89849 | 954676 |
| R90934 | 958474 |
| R91218 | 958758 |
| R91220 | 958760 |
| R91398 | 958938 |
| R91503 | 959043 |
| R91517 | 959057 |
| R91566 | 959106 |
| R91950 | 959490 |
| R92163 | 959703 |
| R92186 | 959726 |
| R92201 | 959741 |
| R92216 | 959756 |
| R92227 | 959767 |
| R92281 | 959821 |
| R92362 | 959902 |
| R92435 | 959975 |
| R92455 | 959995 |
| R92512 | 960052 |
| R92548 | 960088 |
| R92601 | 960141 |
| R92801 | 965155 |
| R92806 | 965160 |
| R92812 | 965166 |
| R92994 | 965348 |
| R93068 | 965422 |
| R93124 | 967290 |
| R93176 | 967342 |
| R93237 | 967403 |
| R93309 | 967475 |
| R93401 | 967567 |
| R93515 | 967681 |
| R93591 | 967757 |
| R93727 | 967893 |
| R93729 | 967895 |
| R93744 | 967910 |
| R93759 | 967925 |
| R93767 | 967933 |
| R93875 | 969270 |
| R94399 | 969794 |
| R94504 | 969899 |
| R94633 | 970028 |
| R95684 | 981344 |
| R95773 | 981433 |
| R95811 | 981471 |
| R95821 | 981481 |
| R95893 | 981553 |
| R95962 | 981622 |
| R96147 | 981807 |
| R96155 | 981815 |
| R96220 | 981880 |
| R96520 | 982180 |
| R96561 | 982221 |
| R96579 | 982239 |
| R96598 | 982258 |
| R96827 | 982487 |
| R96914 | 982574 |
| R96941 | 982601 |
| R97066 | 982726 |
| R97226 | 982886 |
| R98008 | 983668 |
| R98047 | 983707 |
| R98064 | 983724 |
| R98344 | 984004 |
| R98407 | 985119 |
| R98423 | 985135 |
| R98472 | 984989 |
| R98487 | 985004 |
| R98842 | 985443 |
| R98851 | 985452 |
| R99043 | 985644 |
| R99478 | 986079 |
| R99503 | 986104 |
| R99573 | 986174 |
| R99755 | 986356 |
| R99831 | 986432 |
| R39329 | 250893 |
| R41458 | 252252 |
| R42658 | 1679960 |
| R48196 | 258294 |
| R50015 | 260878 |
| R50223 | 260311 |
| R57235 | 298664 |
| R59049 | 299704 |
| R60099 | 300168 |
| R63912 | 399757 |
| R64650 | 407595 |
| R65371 | 410699 |
| R66196 | 239157 |
| R66431 | 435777 |
| R67325 | 455712 |
| R67815 | 460568 |
| R67859 | 239577 |
| R68015 | 461032 |
| R69022 | 516580 |
| R69189 | 545593 |
| R69272 | 546087 |
| S69738 | 545464 |
| S70154 | 546900 |
| S70290 | 546602 |
| S71513 | 240867 |
| S72481 | 632789 |
| S73498 | 688010 |
| S73591 | 688296 |
| S74678 | 241477 |
| S75755 | 861469 |
| S76756 | 913853 |
| S77601 | 998394 |
| S78203 | 999212 |
| S82081 | 1488412 |
| S82240 | 1839516 |
| S94541 | 247146 |
| T00880 | 277361 |
| T02792 | 319308 |
| T04864 | 316023 |
| T05006 | 316161 |
| T05635 | 316785 |
| T06574 | 317723 |
| T06925 | 318074 |
| T06984 | 318133 |
| T07196 | 318345 |
| T08287 | 389315 |
| T08329 | 389357 |
| T08429 | 389457 |
| T08676 | 389704 |
| T10145 | 471494 |
| T10307 | 471656 |
| T18813 | 488232 |
| T18977 | 601020 |
| T19093 | 601136 |
| T27258 | 624974 |
| T28083 | 610181 |
| T28638 | 610736 |
| T28974 | 611072 |
| T29959 | 612057 |
| T30604 | 612702 |
| T30677 | 612775 |
| T30767 | 612865 |
| T31772 | 613870 |
| T32212 | 614310 |
| T34459 | 616557 |
| T34760 | 616858 |
| T35276 | 617374 |
| T36781 | 620598 |
| T37414 | 621231 |
| T40531 | 648153 |
| T40905 | 648489 |
| T41077 | 648648 |
| T41232 | 648790 |
| T43474 | 2758332 |
| T47291 | 649273 |
| T47520 | 649500 |
| T47625 | 649605 |
| T47718 | 649698 |
| T48411 | 650391 |
| T48412 | 650392 |
| T48624 | 650484 |
| T48692 | 650552 |
| T49325 | 651185 |
| T49326 | 651186 |
| T49355 | 651215 |
| T49539 | 651399 |
| T49801 | 651661 |
| T50139 | 651999 |
| T50370 | 652230 |
| T50397 | 652257 |
| T50699 | 652559 |
| T50995 | 652855 |
| T51043 | 652903 |
| T51229 | 653089 |
| T51539 | 653399 |
| T51617 | 653477 |
| T51904 | 653764 |
| T51995 | 653855 |
| T52311 | 654171 |
| T52320 | 654180 |
| T52700 | 654560 |
| T52820 | 654680 |
| T52823 | 654683 |
| T52840 | 654700 |
| T53389 | 655249 |
| T53424 | 655284 |
| T53907 | 655768 |
| T54095 | 655956 |
| T54164 | 656025 |
| T54298 | 656159 |
| T54342 | 656203 |
| T54527 | 656388 |
| T54672 | 656533 |
| T55607 | 657468 |
| T55639 | 657500 |
| T55728 | 657589 |
| T56013 | 657874 |
| T56281 | 658142 |
| T56726 | 658587 |
| T57359 | 659220 |
| T57386 | 659247 |
| T57609 | 659470 |
| T57803 | 659664 |
| T57920 | 659781 |
| T57927 | 659788 |
| T58002 | 659863 |
| T58146 | 660007 |
| T58430 | 660267 |
| T58434 | 660271 |
| T58932 | 660769 |
| T59024 | 660861 |
| T59099 | 660936 |
| T59274 | 661111 |
| T59478 | 661315 |
| T59658 | 661495 |
| T59668 | 661505 |
| T59687 | 661524 |
| T59793 | 661630 |
| T59873 | 661710 |
| T59998 | 661835 |
| T60063 | 661900 |
| T60075 | 661912 |
| T60111 | 661948 |
| T60149 | 661986 |
| T60161 | 661998 |
| T60168 | 662005 |
| T60601 | 663638 |
| T60740 | 663777 |
| T61050 | 664087 |
| T61122 | 664159 |
| T61271 | 664308 |
| T61323 | 664360 |
| T61421 | 664458 |
| T61701 | 664738 |
| T61792 | 665035 |
| T61851 | 665094 |
| T61899 | 665142 |
| T61938 | 665181 |
| T61960 | 665203 |
| T62040 | 665283 |
| T62072 | 665315 |
| T62164 | 665407 |
| T62179 | 665422 |
| T62552 | 666209 |
| T62753 | 666410 |
| T62842 | 666499 |
| T63027 | 666684 |
| T63031 | 666688 |
| T63245 | 667110 |
| T63324 | 667189 |
| T63342 | 667207 |
| T63893 | 667758 |
| T63980 | 667845 |
| T63981 | 667846 |
| T64192 | 668057 |
| T64216 | 668081 |
| T64223 | 668088 |
| T64312 | 668177 |
| T64323 | 668188 |
| T64433 | 668298 |
| T64469 | 673514 |
| T64609 | 673654 |
| T64625 | 673670 |
| T64630 | 673675 |
| T65098 | 674143 |
| T65174 | 674219 |
| T65398 | 674443 |
| T65642 | 674687 |
| T65736 | 674781 |
| T65765 | 674810 |
| T65844 | 674889 |
| T65902 | 674947 |
| T65998 | 675043 |
| T66154 | 675199 |
| T66180 | 675225 |
| T66320 | 675365 |
| T66348 | 675393 |
| T66793 | 676233 |
| T66815 | 676255 |
| T66816 | 676256 |
| T66832 | 676272 |
| T66936 | 676376 |
| T67053 | 676493 |
| T67058 | 676498 |
| T67128 | 676568 |
| T67265 | 676705 |
| T67444 | 678592 |
| T67807 | 678955 |
| T67823 | 678971 |
| T67978 | 679126 |
| T68057 | 679205 |
| T68102 | 679250 |
| T68445 | 679593 |
| T68453 | 679601 |
| T68878 | 680026 |
| T68894 | 680042 |
| T69270 | 680418 |
| T69305 | 680453 |
| T69348 | 680496 |
| T69477 | 680625 |
| T69522 | 680670 |
| T69593 | 680741 |
| T70057 | 681205 |
| T70122 | 681270 |
| T70123 | 681271 |
| T70198 | 681346 |
| T70288 | 681436 |
| T70438 | 681586 |
| T70522 | 681670 |
| T70810 | 685331 |
| T70892 | 685413 |
| T70901 | 685422 |
| T70999 | 685520 |
| T71008 | 685529 |
| T71042 | 685563 |
| T71061 | 685582 |
| T71122 | 685643 |
| T71154 | 685675 |
| T71360 | 685881 |
| T71387 | 685908 |
| T71615 | 686136 |
| T71889 | 686410 |
| T71965 | 686486 |
| T71991 | 686512 |
| T72034 | 686555 |
| T72067 | 686588 |
| T72167 | 686688 |
| T72175 | 686696 |
| T72235 | 686756 |
| T72336 | 686857 |
| T72376 | 686897 |
| T72543 | 689218 |
| T72915 | 689590 |
| T73031 | 689706 |
| T73262 | 689937 |
| T73556 | 690231 |
| T73651 | 690326 |
| T73883 | 690558 |
| T74004 | 690679 |
| T74023 | 690698 |
| T74105 | 690780 |
| T74203 | 690878 |
| T74257 | 690932 |
| T74846 | 691521 |
| T75072 | 691834 |
| T77007 | 694210 |
| T77385 | 694588 |
| T77410 | 694613 |
| T77631 | 694834 |
| T77729 | 694932 |
| T77840 | 695043 |
| T78419 | 696928 |
| T78615 | 697124 |
| T78909 | 697418 |
| T78948 | 697457 |
| T79274 | 697783 |
| T79397 | 697906 |
| T79514 | 698023 |
| T79957 | 698466 |
| T80832 | 703717 |
| T80924 | 703809 |
| T81107 | 703992 |
| T81140 | 704025 |
| T81289 | 704174 |
| T81369 | 704254 |
| T81537 | 704544 |
| T81666 | 704673 |
| T81995 | 705002 |
| T83657 | 711945 |
| T83709 | 711997 |
| T83842 | 712130 |
| T84308 | 712596 |
| T84605 | 712957 |
| T84934 | 713286 |
| T84943 | 713295 |
| T84975 | 713327 |
| T84996 | 713348 |
| T85173 | 713525 |
| T85958 | 714310 |
| T86219 | 714571 |
| T86527 | 714879 |
| T86708 | 715060 |
| T86932 | 715284 |
| T87139 | 715491 |
| T87235 | 715587 |
| T87515 | 715867 |
| T87572 | 715924 |
| T88702 | 717215 |
| T88721 | 717234 |
| T89060 | 717573 |
| T89084 | 717597 |
| T89530 | 718043 |
| T89996 | 718509 |
| T90621 | 719134 |
| T90639 | 719152 |
| T90971 | 722884 |
| T90980 | 722893 |
| T91054 | 722967 |
| T91168 | 723081 |
| T91225 | 723138 |
| T91244 | 723157 |
| T93613 | 726786 |
| T94611 | 728099 |
| T95139 | 733763 |
| T95151 | 733775 |
| T95274 | 733898 |
| T95320 | 733944 |
| T95670 | 734294 |
| T95804 | 734428 |
| T95898 | 734522 |
| T96718 | 735342 |
| T96913 | 735537 |
| T97170 | 735794 |
| T97303 | 746648 |
| T97352 | 746697 |
| T97457 | 746802 |
| T97592 | 746937 |
| T97599 | 746944 |
| T97762 | 747107 |
| T97890 | 747235 |
| T97897 | 747242 |
| T98001 | 747346 |
| T98056 | 747401 |
| T98099 | 747444 |
| T98201 | 747546 |
| T98253 | 747990 |
| T98615 | 748352 |
| T98628 | 748365 |
| T98662 | 748399 |
| T99011 | 748748 |
| T99158 | 748895 |
| T99191 | 748926 |
| T99280 | 749017 |
| T99386 | 749123 |
| T99603 | 749340 |
| T99617 | 749354 |
| 799715 | 749452 |
| T99719 | 749456 |
| T99852 | 749589 |
| T99948 | 749685 |
| U00238 | 404860 |
| U00930 | 405043 |
| U00946 | 405048 |
| U00947 | 405049 |
| U01184 | 440176 |
| U01923 | 460085 |
| U01925 | 460086 |
| U02390 | 409928 |
| U02493 | 407307 |
| U02556 | 413824 |
| U02570 | 450780 |
| U02619 | 414932 |
| U02680 | 451481 |
| U02882 | 433346 |
| U03271 | 595256 |
| U03274 | 468823 |
| U03688 | 501030 |
| U03886 | 458225 |
| U04209 | 434655 |
| U05291 | 450854 |
| U05875 | 463549 |
| U05877 | 463551 |
| U06632 | 458435 |
| U07151 | 460624 |
| U07643 | 467236 |
| U07681 | 706838 |
| U07809 | 469036 |
| U07919 | 995897 |
| U07991 | 468246 |
| U08815 | 508722 |
| U09367 | 487784 |
| U09410 | 488548 |
| U09550 | 1184036 |
| U09559 | 791184 |
| U09564 | 507212 |
| U09813 | 1008454 |
| U09820 | 606832 |
| U09848 | 495567 |
| U10117 | 498909 |
| U10248 | 984280 |
| U10439 | 577169 |
| U10485 | 505685 |
| U12465 | 562073 |
| U12596 | 687238 |
| U12778 | 531390 |
| U12789 | 555832 |
| U12979 | 531394 |
| U13616 | 608024 |
| U13800 | 563171 |
| U13991 | 562076 |
| U14394 | 608128 |
| U14510 | 780373 |
| U14528 | 549987 |
| U14603 | 894158 |
| U14658 | 557469 |
| U14750 | 984955 |
| U14967 | 550014 |
| U14968 | 550016 |
| U14969 | 550018 |
| U14970 | 550020 |
| U14971 | 550022 |
| U15008 | 600747 |
| U15009 | 600749 |
| U15174 | 558845 |
| U15177 | 988207 |
| U15552 | 558457 |
| U16738 | 608516 |
| U16799 | 806753 |
| U16850 | 576644 |
| U16997 | 758419 |
| U17040 | 595945 |
| U17077 | 1000711 |
| U17104 | 609307 |
| U17105 | 576780 |
| U17714 | 4049267 |
| U17743 | 791187 |
| U17838 | 1669774 |
| U17899 | 717053 |
| U18062 | 642794 |
| U18197 | 603073 |
| U18291 | 803230 |
| U18297 | 1117790 |
| U18422 | 604478 |
| U18543 | 758422 |
| U18914 | 790224 |
| U19143 | 914900 |
| U19144 | 914902 |
| U19177 | 726033 |
| U19557 | 1052870 |
| U19769 | 924600 |
| U20157 | 780132 |
| U20770 | 806805 |
| U21049 | 722243 |
| U21090 | 1008457 |
| U21858 | 841307 |
| U21936 | 717118 |
| U22233 | 847723 |
| U22431 | 881345 |
| U22897 | 984286 |
| U24105 | 1638873 |
| U24169 | 1215668 |
| U24704 | 2078477 |
| U25182 | 799380 |
| U25276 | 1184671 |
| U25766 | 808064 |
| U25789 | 808089 |
| U27143 | 862932 |
| U28249 | 897916 |
| U28424 | 1353269 |
| U28964 | 899458 |
| U29091 | 1374791 |
| U30313 | 1050959 |
| U30521 | 963091 |
| U30826 | 1049079 |
| U30897 | 2076716 |
| U31382 | 995916 |
| U31383 | 995918 |
| U31905 | 1353349 |
| U32944 | 1209060 |
| U33760 | 995823 |
| U33821 | 5776544 |
| U34038 | 1041728 |
| U34252 | 1049218 |
| U34994 | 6525311 |
| U36336 | 1209628 |
| U36764 | 1036804 |
| U37143 | 1185451 |
| U37230 | 1574941 |
| U37283 | 1165211 |
| U37518 | 1149557 |
| U37519 | 1051280 |
| U37558 | 1017812 |
| U37689 | 1017822 |
| U37690 | 1017824 |
| U38654 | 4887230 |
| U38784 | 1574947 |
| U38894 | 1589739 |
| U39064 | 1209670 |
| U39318 | 1145690 |
| U39360 | 1066079 |
| U39400 | 1234796 |
| U39657 | 1203817 |
| U39840 | 1066121 |
| U39945 | 1209686 |
| U40272 | 4096802 |
| U40282 | 3150001 |
| U40572 | 1145729 |
| U40671 | 1113938 |
| U40763 | 1117967 |
| U41060 | 1256000 |
| U41515 | 1209723 |
| U41806 | 1145798 |
| U41813 | 1184168 |
| U42068 | 1147738 |
| U42404 | 4096845 |
| U42412 | 1335855 |
| U42594 | 4096861 |
| U43188 | 1420888 |
| U43604 | 1171236 |
| U43701 | 1399085 |
| U43899 | 1556458 |
| U44839 | 1276911 |
| U46309 | 1236451 |
| U46689 | 1870243 |
| U46751 | 3077821 |
| U47414 | 1335886 |
| U47674 | 3860239 |
| U47742 | 1517913 |
| U48857 | 4097194 |
| U49957 | 1537016 |
| U50203 | 1354221 |
| U50733 | 1255187 |
| U50939 | 1314559 |
| U51004 | 1256264 |
| U51134 | 4097252 |
| U51678 | 1915966 |
| U51903 | 1262925 |
| U51920 | 1256819 |
| U51990 | 1805248 |
| U52962 | 4204828 |
| U52969 | 1568614 |
| U53328 | 1431875 |
| U53468 | 1373172 |
| U54558 | 2351377 |
| U54559 | 2351379 |
| U54562 | 2351381 |
| U54831 | 1354506 |
| U54996 | 2661163 |
| U56255 | 1399688 |
| U57052 | 1519039 |
| U57057 | 1654312 |
| U57847 | 1373420 |
| U57877 | 1814225 |
| U58855 | 2745962 |
| U58913 | 4204907 |
| U59435 | 2697004 |
| U59919 | 1633620 |
| U60116 | 4416529 |
| U60205 | 1408205 |
| U60975 | 5030423 |
| U61083 | 4097430 |
| U61232 | 1465771 |
| U61397 | 1518693 |
| U62434 | 1458115 |
| U62740 | 1518041 |
| U62961 | 1519051 |
| U63289 | 1518801 |
| U63542 | 1890646 |
| U63810 | 3282206 |
| U64315 | 1524410 |
| U64820 | 2262194 |
| U65090 | 2462776 |
| U65785 | 1794218 |
| U65928 | 1549382 |
| U66083 | 1519284 |
| U66197 | 1563884 |
| U66469 | 1724072 |
| U66563 | 2228511 |
| U66615 | 1549238 |
| U66616 | 1549240 |
| U67171 | 2326174 |
| U67784 | 1617516 |
| U68140 | 2406564 |
| U68536 | 1698719 |
| U68758 | 4097815 |
| U69263 | 2072789 |
| U69559 | 2731390 |
| U69645 | 1575614 |
| U69668 | 1850341 |
| U70063 | 1743866 |
| U70322 | 1613833 |
| U70323 | 1679683 |
| U70439 | 1698782 |
| U70451 | 1763090 |
| U70730 | 2062370 |
| U70735 | 2360944 |
| U71363 | 1613847 |
| U72041 | 1912457 |
| U72761 | 2102695 |
| U72937 | 6960325 |
| U73642 | 2281066 |
| U73824 | 1857236 |
| U75272 | 1658285 |
| U75329 | 2507612 |
| U75330 | 2507614 |
| U75679 | 1732076 |
| U77396 | 1684871 |
| U77664 | 1885378 |
| U77720 | 2130536 |
| U78516 | 4115908 |
| U78575 | 1743870 |
| U79241 | 1710187 |
| U79254 | 1710206 |
| U79260 | 1710215 |
| U79273 | 1710239 |
| U79278 | 1710247 |
| U79304 | 1710291 |
| U79457 | 4205083 |
| U79716 | 1743884 |
| 079751 | 2257753 |
| U80034 | 1763641 |
| U80040 | 1718501 |
| U80213 | 1857418 |
| U80456 | 2062416 |
| U80669 | 1732377 |
| U80735 | 2565045 |
| U80743 | 2565060 |
| U81001 | 1877436 |
| U81006 | 1737489 |
| U81602 | 1816528 |
| U82226 | 1848270 |
| U82256 | 1773058 |
| U82756 | 2853276 |
| U83115 | 2072424 |
| U83460 | 2315986 |
| U83857 | 2623760 |
| U84138 | 2801404 |
| U84720 | 1903455 |
| U85658 | 2058552 |
| U87166 | 3165428 |
| U88666 | 1857943 |
| U89436 | 2665518 |
| U89505 | 2078528 |
| U90028 | 2745975 |
| U90144 | 1899195 |
| U90548 | 2062697 |
| U90550 | 2062701 |
| U90551 | 2062703 |
| U90904 | 1913382 |
| U90907 | 1913886 |
| U90909 | 1913889 |
| U90915 | 1913895 |
| U90916 | 1913897 |
| U90919 | 1913900 |
| U90942 | 4099879 |
| U91321 | 2951946 |
| U92458 | 1935040 |
| U92544 | 4099968 |
| U94586 | 1946691 |
| U95646 | 4530026 |
| U95825 | 4559409 |
| U96721 | 2454272 |
| U96759 | 2738243 |
| U96915 | 2108209 |
| U97280 | 2689607 |
| U97519 | 2213812 |
| V00478 | 28244 |
| V00594 | 37120 |
| W00471 | 1271880 |
| W00556 | 1271975 |
| W00890 | 1272936 |
| W00895 | 1272875 |
| WO1011 | 1272990 |
| W01113 | 1273161 |
| W01211 | 1273190 |
| W0P739 | 1273719 |
| W01800 | 1274001 |
| W02073 | 1274053 |
| W02256 | 1274254 |
| W02333 | 1274331 |
| W02342 | 1274349 |
| W02426 | 1274612 |
| W02483 | 1274481 |
| W02575 | 1274859 |
| W02978 | 1274985 |
| W03029 | 1275007 |
| W03170 | 1275188 |
| W03191 | 1275209 |
| W03366 | 1275317 |
| W03386 | 1275249 |
| W03471 | 1275335 |
| W03687 | 1275532 |
| W04391 | 1276100 |
| W04240 | 1276139 |
| W04380 | 1276288 |
| W04421 | 1276329 |
| W04588 | 1277455 |
| W05026 | 1277746 |
| W05033 | 1277773 |
| W05240 | 1277972 |
| W05402 | 1278133 |
| W05406 | 1278137 |
| W05463 | 1278185 |
| W05472 | 1278204 |
| W05628 | 1278497 |
| W05661 | 1278393 |
| W06897 | 1281120 |
| W07109 | 1281266 |
| W07165 | 1281176 |
| W07378 | 1281379 |
| W15195 | 1289576 |
| W15207 | 1289588 |
| W15263 | 1289653 |
| W15277 | 1289667 |
| W15305 | 1289705 |
| W15319 | 1289769 |
| W15477 | 1289996 |
| W15554 | 1289984 |
| W16424 | 1289598 |
| W16659 | 1291258 |
| W16724 | 1291124 |
| W16832 | 1291220 |
| W19519 | 1295437 |
| W19757 | 1295803 |
| W19759 | 1295805 |
| W19899 | 1295768 |
| W19970 | 1295840 |
| W20438 | 1295055 |
| W20458 | 1295228 |
| W20462 | 1295061 |
| W20486 | 1295075 |
| W21047 | 1297923 |
| W21373 | 1298425 |
| W21894 | 1298776 |
| W22330 | 1299177 |
| W23631 | 1300446 |
| W23758 | 1300592 |
| W23847 | 1300729 |
| W24228 | 1301190 |
| W24646 | 1301548 |
| W24708 | 1301525 |
| W25008 | 1302863 |
| W25081 | 1302985 |
| W25142 | 1303036 |
| W25202 | 1303076 |
| W25222 | 1303086 |
| W25970 | 1306300 |
| W26008 | 1306275 |
| W26372 | 1306927 |
| W26113 | 1307112 |
| W26780 | 1305999 |
| W27182 | 1306698 |
| W27229 | 1306745 |
| W27440 | 1307307 |
| W27484 | 1307288 |
| W27526 | 1307330 |
| W27968 | 1307916 |
| W28466 | 1308414 |
| W28596 | 1308544 |
| W28729 | 1308677 |
| W28994 | 1308960 |
| W30686 | 1311872 |
| W30832 | 1311889 |
| W31074 | 1312064 |
| W31167 | 1312159 |
| W31255 | 1312247 |
| W31389 | 1312380 |
| W31725 | 1312718 |
| W31819 | 1312809 |
| W31992 | 1313004 |
| W32156 | 1313167 |
| W32272 | 1313264 |
| W32303 | 1313314 |
| W32408 | 1313398 |
| W32729 | 1313720 |
| W35236 | 1317142 |
| W35301 | 1317207 |
| W35398 | 1317334 |
| W37112 | 1318961 |
| W37306 | 1319037 |
| W37322 | 1318917 |
| W37343 | 1318976 |
| W37418 | 1319012 |
| W37447 | 1319061 |
| W37532 | 1319146 |
| W37602 | 1319216 |
| W37614 | 1319228 |
| W37753 | 1319347 |
| W37778 | 1319589 |
| W37779 | 1319590 |
| W37862 | 1319526 |
| W37945 | 1319539 |
| W38407 | 1320008 |
| W38679 | 1320384 |
| W38726 | 1320433 |
| W38913 | 1320619 |
| W38956 | 1320664 |
| W39053 | 1320762 |
| W39249 | 1321040 |
| W39298 | 1321015 |
| W39343 | 1321069 |
| W39609 | 1321357 |
| W40294 | 1324114 |
| W42653 | 1327113 |
| W42812 | 1327272 |
| W42913 | 1327404 |
| W42996 | 1327496 |
| W43028 | 1327528 |
| W44338 | 1329897 |
| W44508 | 1330009 |
| W44623 | 1330172 |
| W44766 | 1328848 |
| W44852 | 1328942 |
| W44889 | 1328989 |
| W44911 | 1329204 |
| W44938 | 1329019 |
| W44954 | 1329035 |
| W45014 | 1329095 |
| W45148 | 1329239 |
| W45179 | 1329270 |
| W45265 | 1329366 |
| W45285 | 1329387 |
| W45330 | 1329422 |
| W45403 | 1329484 |
| W45623 | 1329704 |
| W46577 | 1331242 |
| W46900 | 1331538 |
| W46985 | 1331623 |
| W47000 | 1331638 |
| W47254 | 1332071 |
| W47364 | 1332003 |
| W47487 | 1332274 |
| W47576 | 1332227 |
| W47585 | 1332236 |
| W48700 | 1336878 |
| W48762 | 1336911 |
| W48827 | 1336957 |
| W49563 | 1337820 |
| W49583 | 1337867 |
| W49619 | 1338087 |
| W49672 | 1337927 |
| W51795 | 1349846 |
| W51959 | 1349213 |
| W52208 | 1349338 |
| W52341 | 1349531 |
| W52355 | 1349507 |
| W52896 | 1350320 |
| W56069 | 1357959 |
| W56077 | 1357967 |
| W56388 | 1358278 |
| W56395 | 1358285 |
| W56523 | 1358409 |
| W56718 | 1358575 |
| W56760 | 1358626 |
| W57712 | 1364499 |
| W57818 | 1364533 |
| W58013 | 1364745 |
| W58191 | 1364904 |
| W58209 | 1364922 |
| W58291 | 1365003 |
| W58562 | 1365294 |
| W58683 | 1365415 |
| W58731 | 1365481 |
| W60015 | 1366774 |
| W60040 | 1366868 |
| W60288 | 1367047 |
| W60414 | 1367398 |
| W60565 | 1367343 |
| W60795 | 1367639 |
| W60845 | 1367603 |
| W60894 | 1367779 |
| W61042 | 1367839 |
| W61049 | 1367809 |
| W61215 | 1367982 |
| W61361 | 1366798 |
| W63659 | 1371240 |
| W63676 | 1371257 |
| W63776 | 1371377 |
| W63789 | 1371390 |
| W63793 | 1371127 |
| W65292 | 1373618 |
| W67309 | 1376179 |
| W67735 | 1376626 |
| W68028 | 1376897 |
| W68141 | 1377226 |
| W68162 | 1377032 |
| W68220 | 1377158 |
| W68291 | 1377389 |
| W68396 | 1377267 |
| W69160 | 1378441 |
| W69213 | 1378473 |
| W69271 | 1378746 |
| W69460 | 1378722 |
| W69649 | 1379218 |
| W69741 | 1379072 |
| W69806 | 1379086 |
| W69960 | 1379240 |
| W70074 | 1379335 |
| W70128 | 1379614 |
| W70147 | 1379408 |
| W70234 | 1379503 |
| W70242 | 1379511 |
| W70264 | 1379553 |
| W70343 | 1379643 |
| W71990 | 1382431 |
| W72051 | 1382321 |
| W72231 | 1382854 |
| W72263 | 1382866 |
| W72310 | 1382933 |
| W72431 | 1382386 |
| W72437 | 1382363 |
| W72556 | 1382193 |
| W72666 | 1382486 |
| W72671 | 1382491 |
| W72679 | 1382499 |
| W72729 | 1382764 |
| W72749 | 1382727 |
| W72798 | 1382911 |
| W72838 | 1382814 |
| W72881 | 1383094 |
| W72920 | 1383055 |
| W72973 | 1383116 |
| W73022 | 1383175 |
| W73144 | 1383279 |
| W73290 | 1383425 |
| W73377 | 1383510 |
| W73474 | 1383606 |
| W73523 | 1383656 |
| W73588 | 1383926 |
| W73634 | 1383768 |
| W73724 | 1383916 |
| W73748 | 1384017 |
| W73753 | 1383898 |
| W73781 | 1383944 |
| W73790 | 1383953 |
| W73811 | 1383964 |
| W73817 | 1383970 |
| W73889 | 1382284 |
| W73892 | 1382287 |
| W73994 | 1384641 |
| W74046 | 1384267 |
| W74133 | 1384315 |
| W74216 | 1384396 |
| W74362 | 1384668 |
| W74377 | 1384792 |
| W74462 | 1384749 |
| W74471 | 1384758 |
| W74533 | 1384805 |
| W74633 | 1384846 |
| W74653 | 1384866 |
| W74673 | 1384905 |
| W74696 | 1384919 |
| W74725 | 1384948 |
| W74802 | 1385053 |
| W76133 | 1386357 |
| W76134 | 1386358 |
| W76319 | 1386553 |
| W76331 | 1386575 |
| W76603 | 1386848 |
| W77826 | 1388360 |
| W78168 | 1388702 |
| W78775 | 1389312 |
| W78967 | 1389196 |
| W79069 | 1390572 |
| W79340 | 1390472 |
| W79445 | 1390696 |
| W79499 | 1390759 |
| W79525 | 1390785 |
| W79647 | 1390075 |
| W79668 | 1390057 |
| W79717 | 1390125 |
| W79779 | 1390207 |
| W79834 | 1390242 |
| W80447 | 1391503 |
| W80496 | 1391611 |
| W80611 | 1391698 |
| W80666 | 1391704 |
| W80673 | 1391711 |
| W80730 | 1391748 |
| W80741 | 1391759 |
| W80791 | 1391809 |
| W81084 | 1392136 |
| W81117 | 1391616 |
| W81118 | 1391617 |
| W81124 | 1391623 |
| W81196 | 1392235 |
| W81432 | 1392472 |
| W81526 | 1392555 |
| W84471 | 1395583 |
| W84486 | 1395617 |
| W84560 | 1395672 |
| W84627 | 1395738 |
| W84716 | 1395825 |
| W84751 | 1395870 |
| W84774 | 1395894 |
| W84778 | 1395898 |
| W84815 | 1396014 |
| W85697 | 1398344 |
| W85709 | 1398150 |
| W85764 | 1398425 |
| W85832 | 1398262 |
| W85878 | 1398307 |
| W85881 | 1398310 |
| W85931 | 1398520 |
| W86075 | 1398524 |
| W86124 | 1398554 |
| W86160 | 1398639 |
| W86183 | 1398623 |
| W86196 | 1398746 |
| W86202 | 1398642 |
| W86214 | 1398827 |
| W86282 | 1398720 |
| W86378 | 1398196 |
| W86466 | 1400213 |
| W86653 | 1400529 |
| W86779 | 1400507 |
| W86823 | 1400622 |
| W86832 | 1400561 |
| W86860 | 1400589 |
| W86987 | 1400744 |
| W87281 | 1401476 |
| W87312 | 1401377 |
| W87509 | 1401656 |
| W87522 | 1401728 |
| W87710 | 1401764 |
| W87801 | 1401886 |
| W87823 | 1401898 |
| W87826 | 1401901 |
| W87865 | 1401930 |
| W87891 | 1401976 |
| W88623 | 1404173 |
| W88720 | 1404202 |
| W88737 | 1404219 |
| W88786 | 1404477 |
| W88792 | 1404264 |
| W88823 | 1404295 |
| W88848 | 1404340 |
| W88856 | 1404408 |
| W88942 | 1403848 |
| W88999 | 1403885 |
| W89057 | 1404082 |
| W89074 | 1404167 |
| W90036 | 1406076 |
| W90067 | 1406077 |
| W90128 | 1406118 |
| W90164 | 1406303 |
| W90224 | 1406214 |
| W90323 | 1406703 |
| W90364 | 1406602 |
| W90381 | 1406138 |
| W90519 | 1406324 |
| W90520 | 1406325 |
| W90529 | 1406395 |
| W90705 | 1406651 |
| W90732 | 1406678 |
| W91890 | 1424282 |
| W92011 | 1424374 |
| W92263 | 1424628 |
| W92278 | 1424663 |
| W92431 | 1424815 |
| W92798 | 1421951 |
| W92964 | 1422116 |
| W92970 | 1422141 |
| W93147 | 1422516 |
| W93315 | 1422457 |
| W93379 | 1422501 |
| W93483 | 1422752 |
| W93500 | 1422837 |
| W93523 | 1422644 |
| W93585 | 1422706 |
| W93638 | 1422779 |
| W93655 | 1422845 |
| W93682 | 1422804 |
| W93709 | 1422851 |
| W93717 | 1422859 |
| W93718 | 1423061 |
| W94009 | 1423130 |
| W94121 | 1423244 |
| W94289 | 1423410 |
| W94316 | 1423437 |
| W94398 | 1423529 |
| W94486 | 1423765 |
| W94591 | 1423713 |
| W94647 | 1423829 |
| W95001 | 1424170 |
| W95050 | 1424398 |
| W95082 | 1424200 |
| W95118 | 1424236 |
| W95279 | 1425205 |
| W95409 | 1425324 |
| W95414 | 1425329 |
| W95757 | 1425712 |
| W96707 | 1426014 |
| W96114 | 1426021 |
| W96134 | 1426060 |
| W96155 | 1426061 |
| W96187 | 1426093 |
| W96189 | 1426095 |
| W96197 | 1426103 |
| W96214 | 1426140 |
| W96216 | 1426142 |
| W96273 | 1426180 |
| W96280 | 1426187 |
| W99251 | 1432178 |
| X00351 | 23251 |
| X00497 | 32130 |
| X00630 | 11585 |
| X00632 | 10627 |
| X01037 | 36086 |
| X01630 | 28871 |
| X01742 | 35324 |
| X02422 | 33344 |
| X02457 | 32153 |
| X02530 | 33917 |
| X02544 | 24444 |
| X02596 | 29420 |
| X02761 | 31396 |
| X02875 | 35124 |
| X02974 | 10873 |
| X03559 | 28939 |
| X04098 | 28338 |
| X04106 | 35327 |
| X04125 | 58114 |
| X04297 | 28926 |
| X04318 | 52847 |
| X04325 | 31646 |
| X04407 | 29934 |
| X04408 | 31914 |
| X04412 | 35447 |
| X04470 | 28638 |
| X04503 | 36490 |
| X04526 | 31667 |
| X04571 | 31120 |
| X04697 | 31991 |
| X04707 | 31208 |
| X05276 | 37201 |
| X05332 | 35740 |
| X05345 | 32457 |
| X05606 | 28930 |
| X05908 | 34387 |
| X06272 | 30865 |
| X06537 | 34284 |
| X06617 | 36143 |
| X06747 | 36101 |
| X06834 | 5941 |
| X07270 | 32485 |
| X07429 | 29524 |
| X07466 | 31769 |
| X07695 | 34072 |
| X07767 | 35478 |
| X07819 | 35798 |
| X07820 | 36628 |
| X07876 | 33970 |
| X12433 | 32451 |
| X12451 | 29714 |
| X12597 | 32326 |
| X12654 | 35908 |
| X12791 | 36112 |
| X13238 | 1200056 |
| X13425 | 31590 |
| X13482 | 37546 |
| X13585 | 37579 |
| X13839 | 28329 |
| X14420 | 30057 |
| X14608 | 296365 |
| X15183 | 32487 |
| X15187 | 37260 |
| X15653 | 37598 |
| X15722 | 31824 |
| X15729 | 38317 |
| X15759 | 12583 |
| X15822 | 30146 |
| X15880 | 30029 |
| X15949 | 33966 |
| X16896 | 33800 |
| X16940 | 36502 |
| X17206 | 34391 |
| X17620 | 35067 |
| X51602 | 31431 |
| X51668 | 6641 |
| X52022 | 3127925 |
| X52174 | 28321 |
| X52235 | 34372 |
| X52268 | 44100 |
| X52882 | 311380 |
| X52943 | 28912 |
| X53280 | 29504 |
| X53605 | 35683 |
| X53777 | 34198 |
| X53793 | 28383 |
| X53961 | 34415 |
| X54199 | 31641 |
| X54326 | 31957 |
| X54473 | 311347 |
| X54941 | 29976 |
| X55525 | 30101 |
| X55654 | 14015 |
| X56351 | 28582 |
| X56415 | 28410 |
| X56807 | 30507 |
| X56932 | 23690 |
| X56958 | 4803677 |
| X56998 | 37564 |
| X56999 | 37568 |
| X57025 | 33007 |
| X57351 | 311373 |
| X57352 | 311374 |
| X57398 | 35526 |
| X57500 | 35037 |
| X57766 | 456256 |
| X57810 | 33717 |
| X57812 | 33723 |
| X58141 | 28381 |
| X59131 | 3776087 |
| X59352 | 37214 |
| X59405 | 34508 |
| X59406 | 34509 |
| X59618 | 36154 |
| X59834 | 31830 |
| X59841 | 35314 |
| X59892 | 30820 |
| X60036 | 38261 |
| X60111 | 34768 |
| X60221 | 509290 |
| X60489 | 31099 |
| X60656 | 31134 |
| X60708 | 35335 |
| X61100 | 38078 |
| X61970 | 296739 |
| X62048 | 499071 |
| X62534 | 32332 |
| X62744 | 36062 |
| X63380 | 36168 |
| X63422 | 12585 |
| X63423 | 12587 |
| X63432 | 28335 |
| X63563 | 36121 |
| X63564 | 36123 |
| X63679 | 37264 |
| X63753 | 36545 |
| X64330 | 28934 |
| X64707 | 29382 |
| X64838 | 35998 |
| X64875 | 398163 |
| X65550 | 415818 |
| X65644 | 38259 |
| X65923 | 31302 |
| X66276 | 36500 |
| X66534 | 31683 |
| X66785 | 30489 |
| X67098 | 475908 |
| X67698 | 37476 |
| X67951 | 287640 |
| X68277 | 29980 |
| X68560 | 38417 |
| X68684 | 296460 |
| X69086 | 34811 |
| X69111 | 32294 |
| X69117 | 312394 |
| X69711 | 296534 |
| X70326 | 38434 |
| X70394 | 468707 |
| X71087 | 288396 |
| X71129 | 297901 |
| X72727 | 460788 |
| X73314 | 402618 |
| X73608 | 793844 |
| X73874 | 439271 |
| X73902 | 452754 |
| X74070 | 395086 |
| X74331 | 510407 |
| X74801 | 671526 |
| X75304 | 405714 |
| X75535 | 1644300 |
| X75546 | 453156 |
| X75593 | 452319 |
| X75861 | 456258 |
| X76013 | 531595 |
| X76057 | 416016 |
| X76105 | 434844 |
| X76180 | 452649 |
| X76302 | 431952 |
| X76538 | 434976 |
| X76578 | 435144 |
| X76732 | 2706486 |
| X76770 | 556782 |
| X76771 | 1905802 |
| X77196 | 704462 |
| X77548 | 469145 |
| X77753 | 1524102 |
| X78136 | 460772 |
| X78137 | 460770 |
| X78520 | 854101 |
| X78627 | 607129 |
| X78926 | 498724 |
| X78933 | 498720 |
| X79201 | 531105 |
| X79389 | 510904 |
| X79535 | 496886 |
| X79888 | 780240 |
| X80676 | 1240060 |
| X80695 | 619490 |
| X80754 | 577778 |
| X80822 | 527577 |
| X80910 | 531475 |
| X80916 | 531367 |
| X81109 | 535057 |
| X81198 | 773574 |
| X81695 | 940515 |
| X81789 | 551449 |
| X81836 | 577052 |
| X81889 | 1702923 |
| X81900 | 2274973 |
| X82125 | 1310670 |
| X82157 | 758065 |
| X82321 | 1617117 |
| X83544 | 1089849 |
| X83973 | 639692 |
| X84908 | 1502344 |
| X85038 | 728715 |
| X85129 | 854125 |
| X85134 | 755749 |
| X85337 | 1262344 |
| X85372 | 806563 |
| X85373 | 806565 |
| X86098 | 899293 |
| X87176 | 1050516 |
| X87241 | 1107686 |
| X87388 | 1304319 |
| X87397 | 1304370 |
| X87624 | 8052258 |
| X87949 | 1143491 |
| X89652 | 902619 |
| X89673 | 902329 |
| X89852 | 1050424 |
| X90444 | 1066232 |
| X90568 | 1212991 |
| X90583 | 1071680 |
| X90870 | 1103676 |
| X91257 | 1050526 |
| X91484 | 987789 |
| X92098 | 1212964 |
| X92689 | 1296629 |
| X92845 | 2344811 |
| X92972 | 1945270 |
| X93036 | 1085025 |
| X94323 | 1213612 |
| X94754 | 1702931 |
| X94910 | 3413292 |
| X95240 | 1262818 |
| X95325 | 1167837 |
| X95384 | 2792003 |
| X95677 | 1491701 |
| X97065 | 1296665 |
| X97324 | 1806039 |
| X97335 | 1507823 |
| X97674 | 1877214 |
| X97675 | 1834512 |
| X97676 | 1313923 |
| X97677 | 1313925 |
| X97772 | 1944613 |
| X97790 | 1806089 |
| X97841 | 1322145 |
| X97888 | 1666659 |
| X98263 | 1770461 |
| X98296 | 1666074 |
| X98608 | 1419345 |
| X98743 | 1498228 |
| X98801 | 1419566 |
| X98891 | 1420872 |
| X98901 | 3928200 |
| X99098 | 1592819 |
| X99585 | 1770518 |
| X99920 | 1694827 |
| Y00052 | 30308 |
| Y00281 | 36052 |
| Y00282 | 36048 |
| Y00345 | 35569 |
| Y00361 | 37575 |
| Y00503 | 34038 |
| Y00711 | 34328 |
| Y00815 | 34266 |
| Y00971 | 35699 |
| Y07593 | 1881446 |
| Y07968 | 1552327 |
| Y08319 | 1922312 |
| Y08915 | 1877201 |
| Y08991 | 1817583 |
| Y09188 | 2230868 |
| Y09267 | 1834492 |
| Y09305 | 1669684 |
| Y09615 | 1707506 |
| Y09836 | 1743253 |
| Y10313 | 2706510 |
| Y10351 | 3021391 |
| Y11164 | 2145440 |
| Y11395 | 2894085 |
| Y11435 | 2910996 |
| Y12065 | 2230877 |
| Y12781 | 3021408 |
| Y12860 | 2808530 |
| Y13286 | 2853173 |
| Y13323 | 5042231 |
| Y13834 | 5327058 |
| Y13936 | 2315201 |
| Y14738 | 2765426 |
| Y15286 | 2584788 |
| Y15906 | 5327056 |
| Y16132 | 2765526 |
| Y16241 | 3378195 |
| Y16414 | 2924334 |
| Y16610 | 3273088 |
| Y18314 | 5457356 |
| Z11227 | 34926 |
| Z11241 | 34939 |
| Z11338 | 36236 |
| Z11491 | 41072 |
| Z11531 | 31103 |
| Z11692 | 31107 |
| Z11793 | 36425 |
| Z13009 | 31072 |
| Z14244 | 30150 |
| Z14392 | 6057 |
| Z14468 | 6133 |
| Z15085 | 20243 |
| Z15124 | 7226 |
| Z15132 | 65134 |
| Z15187 | 288856 |
| Z16412 | 48235 |
| Z16695 | 23264 |
| Z17335 | 10476 |
| Z17710 | 16894 |
| Z17864 | 30786 |
| Z18356 | 30768 |
| Z18538 | 28711 |
| Z19002 | 38517 |
| Z19471 | 29313 |
| Z20613 | 27358 |
| Z21507 | 38521 |
| Z22534 | 402184 |
| Z22548 | 438068 |
| Z22658 | 297411 |
| Z22968 | 312141 |
| Z23064 | 3256006 |
| Z24611 | 394980 |
| Z24724 | 505034 |
| Z24862 | 395500 |
| Z24892 | 395530 |
| Z24893 | 395531 |
| Z25142 | 395761 |
| Z25169 | 395808 |
| Z26248 | 940510 |
| Z26317 | 416177 |
| Z27237 | 416010 |
| Z27248 | 454317 |
| Z27252 | 416498 |
| Z29067 | 479172 |
| Z29330 | 483537 |
| Z30311 | 456749 |
| Z31696 | 479156 |
| Z33428 | 485886 |
| Z33445 | 486641 |
| Z33526 | 3702572 |
| Z33526 | 488404 |
| Z33579 | 488668 |
| Z33658 | 488810 |
| Z33671 | 493260 |
| Z33949 | 496436 |
| Z34217 | 498655 |
| Z34975 | 575653 |
| Z36531 | 535184 |
| Z36715 | 531522 |
| Z36731 | 531581 |
| Z36785 | 533902 |
| Z36819 | 533933 |
| Z37986 | 780262 |
| Z41241 | 566997 |
| Z43146 | 570729 |
| Z43304 | 570892 |
| Z43843 | 572935 |
| Z44402 | 573532 |
| Z44556 | 573700 |
| Z45274 | 574484 |
| Z46212 | 574077 |
| Z46606 | 575250 |
| Z46973 | 987947 |
| Z47087 | 860989 |
| Z47553 | 623241 |
| Z48042 | 662993 |
| Z48054 | 732797 |
| Z48605 | 727224 |
| Z48633 | 1165102 |
| Z50749 | 1085027 |
| Z63584 | 1035962 |
| Z68747 | 1546899 |
| Z69043 | 2398656 |
| Z73900 | 1340069 |
| Z74615 | 1418927 |
| Z78396 | 1495169 |
| Z82022 | 2239118 |
| Z83843 | 2578095 |
| Z93020 | 3288048 |
| Z94388 | 1945873 |

What is claimed is:

1. A method of assessing whether a patient is afflicted with prostate cancer, the method comprising comparing:
a) the level of expression of a marker in a patient sample, wherein the marker is selected from the group consisting of the markers listed in Tables 1-1 to 6, and
b) the normal level of expression of the marker in a control non-prostate cancer sample,
wherein a significant difference between the level of expression of the marker in the patient sample and the normal level is an indication that the patient is afflicted with prostate cancer.

2. The method of claim 1, wherein the marker corresponds to a secreted protein.

3. The method of claim 1, wherein the marker corresponds to a transcribed polynucleotide or portion thereof, wherein the polynucleotide comprises the marker.

4. The method of claim 1, wherein the sample comprises cells obtained from the patient.

5. The method of claim 4, wherein the sample is a prostate tissue sample.

6. The method of claim 4, wherein the cells are in a fluid selected from the group consisting of blood fluids, semen, prostate fluid, lymph and urine.

7. The method of claim 1, wherein the level of expression of the marker in the sample is assessed by detecting the presence in the sample of a protein or protein fragment corresponding to the marker.

8. The method of claim 7, wherein the presence of the protein or protein fragment is detected using a reagent which specifically binds with the protein or protein fragment.

9. The method of claim 8, wherein the reagent is selected from the group consisting of an antibody, an antibody derivative, and an antibody fragment.

10. The method of claim 1, wherein the level of expression of the marker in the sample is assessed by detecting the presence in the sample of a transcribed polynucleotide or portion thereof, wherein the transcribed polynucleotide comprises the marker.

11. The method of claim 10, wherein the transcribed polynucleotide is an mRNA.

12. The method of claim 10, wherein the transcribed polynucleotide is a cDNA.

13. The method of claim 10, wherein the step of detecting further comprises amplifying the transcribed polynucleotide.

14. The method of claim 1, wherein the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions.

15. The method of claim 1, wherein the level of expression of the marker in the sample differs from the normal level of expression of the marker in a patient not afflicted with prostate cancer by a factor of at least about 2.

16. The method of claim 1, wherein the level of expression of the marker in the sample differs from the normal level of expression of the marker in a patient not afflicted with prostate cancer by a factor of at least about 5.

17. The method of claim 1, comprising comparing:
a) the level of expression in the sample of each of a plurality of markers independently selected from the markers listed in Tables 1-1 to 6, and
b) the normal level of expression of each of the plurality of markers in samples of the same type obtained from control humans not afflicted with prostate cancer,
wherein the level of expression of more than one of the markers is significantly altered, relative to the corresponding normal levels of expression of the markers, is an indication that the patient is afflicted with prostate cancer.

18. The method of claim 17, wherein the level of expression of each of the markers is significantly altered, relative to the corresponding normal levels of expression of the markers, is an indication that the patient is afflicted with prostate cancer.

19. The method of claim 17, wherein the plurality comprises at least three of the markers.

20. The method of claim 17, wherein the plurality comprises at least five of the markers.

21. A method for monitoring the progression of prostate cancer in a patient, the method comprising:
a) detecting in a patient sample at a first point in time, the expression of a marker, wherein the marker is selected from the group consisting of the markers listed in Tables 1-1 to 6;
b) repeating step a) at a subsequent point in time; and
c) comparing the level of expression detected in steps a) and b), and therefrom monitoring the progression of prostate cancer.

22. The method of claim 21, wherein the marker corresponds to a secreted protein.

23. The method of claim 21, wherein the marker corresponds to a transcribed polynucleotide or portion thereof, wherein the polynucleotide comprises the marker.

24. The method of claim 21, wherein the sample comprises cells obtained from the patient.

25. The method of claim 24, wherein the patient sample is a prostate tissue sample.

26. The method of claim 21, wherein between the first point in time and the subsequent point in time, the patient has undergone surgery to remove prostate tissue.

27. A method of assessing the efficacy of a test compound for inhibiting prostate cancer in a patient, the method comprising comparing:
a) expression of a marker in a first sample obtained from the patient and exposed to the test compound, wherein the marker is selected from the group consisting of the markers listed in Tables 1-1 to 6, and
b) expression of the marker in a second sample obtained from the patient, wherein the sample is not exposed to the test compound,
wherein a significantly lower level of expression of the marker in the first sample, relative to the second sample, is an indication that the test compound is efficacious for inhibiting prostate cancer in the patient.

28. The method of claim 27, wherein the first and second samples are portions of a single sample obtained from the patient.

29. The method of claim 27, wherein the first and second samples are portions of pooled samples obtained from the patient.

30. A method of assessing the efficacy of a therapy for inhibiting prostate cancer in a patient, the method comprising comparing:
a) expression of a marker in the first sample obtained from the patient prior to providing at least a portion of the therapy to the patient, wherein the marker is selected from the group consisting of the markers listed in Tables 1-1 to 6, and
b) expression of the marker in a second sample obtained from the patient following provision of the portion of the therapy,
wherein a significantly lower level of expression of the marker in the second sample, relative to the first sample, is an indication that the therapy is efficacious for inhibiting prostate cancer in the patient.

31. A method of selecting a composition for inhibiting prostate cancer in a patient, the method comprising:
a) obtaining a sample comprising cancer cells from the patient;
b) separately exposing aliquots of the sample in the presence of a plurality of test compositions;
c) comparing expression of a marker in each of the aliquots, wherein the marker is selected from the group consisting of the markers listed in Tables 1-1 to 6; and
d) selecting one of the test compositions which alters the level of expression of the marker in the aliquot containing that test composition, relative to other test compositions.

32. A method of inhibiting prostate cancer in a patient, the method comprising:
a) obtaining a sample comprising cancer cells from the patient;
b) separately maintaining aliquots of the sample in the presence of a plurality of test compositions;
c) comparing expression of a marker in each of the aliquots, wherein the marker is selected from the group consisting of the markers listed in Tables 1-1 to 6; and
d) administering to the patient at least one of the test compositions which alters the level of expression of the marker in the aliquot containing that test composition, relative to other test compositions.

33. A kit for assessing whether a patient is afflicted with prostate cancer, the kit comprising a marker selected from the group consisting of the markers listed in Tables 1-1 to 6.

34. A kit for assessing the presence of prostate cancer cells, the kit comprising a nucleic acid probe wherein the probe specifically binds with a transcribed polynucleotide corresponding to a marker selected from the group consisting of the markers listed in Tables 1-1 to 6.

35. A kit for assessing the suitability of each of a plurality of compounds for inhibiting prostate cancer in a patient, the kit comprising:
a) the plurality of compounds; and
b) a reagent for assessing expression of a marker selected from the group consisting of the markers listed in Tables 1-1 to 6.

36. A method of making an isolated hybridoma which produces an antibody useful for assessing whether a patient is afflicted with prostate cancer, the method comprising:
isolating a protein or protein fragment corresponding to a marker selected from the group consisting of the markers listed in Tables 1-1 to 6;
immunizing a mammal using the isolated protein or protein fragment;
isolating splenocytes from the immunized mammal;
fusing the isolated splenocytes with an immortalized cell line to form hybridomas; and
screening individual hybridomas for production of an antibody which specifically binds with the protein or protein fragment to isolate the hybridoma.

37. An antibody produced by a hybridoma made by the method of claim 36.

38. A kit for assessing the presence of human prostate cancer cells, the kit comprising an antibody, wherein the antibody specifically binds with a protein or protein fragment corresponding to a marker selected from the group consisting of the markers listed in Tables 1-1 to 6.

39. A method of assessing the prostate cell carcinogenic potential of a test compound, the method comprising:
a) maintaining separate aliquots of prostate cells in the presence and absence of the test compound; and
b) comparing expression of a marker in each of the aliquots, wherein the marker is selected from the group consisting of the markers listed in Tables 1-1 to 6,
wherein a significantly altered level of expression of the marker in the aliquot maintained in the presence of the test compound, relative to the aliquot maintained in the absence of the test compound, is an indication that the test compound possesses human prostate cell carcinogenic potential.

40. A kit for assessing the prostate cell carcinogenic potential of a test compound, the kit comprising prostate cells and a reagent for assessing expression of a marker, wherein the marker is selected from the group consisting of the markers listed in Tables 1-1 to 6.

41. A method of inhibiting prostate cancer in a patient at risk for developing prostate cancer, the method comprising inhibiting expression of a gene corresponding to a marker selected from the markers listed in Tables 1-1 to 6, wherein the gene is overexpressed in prostate cancer.

42. The method of claim 41, further comprising the step of providing to cells of the patient an antisense oligonucleotide complementary to a polynucleotide corresponding to a marker selected from the markers listed in Tables 1-1 to 6.

43. A method of inhibiting prostate cancer in a patient at risk for developing prostate cancer, the method comprising increasing expression of a gene corresponding to a marker selected from the markers listed in Tables 1-1 to 6, wherein the gene is underexpressed in prostate cancer or expressed in normal prostate tissue.

44. A method for determining whether prostate cancer has metastasized in a patient, the method comprising comparing:
a) the level of expression of a marker in a patient smaple, wherein the marker is selected from the group consisting of the markers listed in Tables 1-1 to 6, and
b) the normal level or non-metastatic level of expression of the marker in a control sample
wherein a significant difference between the level of expression in the patient sample and the normal level or non-metastatic level is an indication that the prostate cancer has mestastasized.

45. The method of claim 44, wherein the marker corresponds to a secreted protein.

46. The method of claim 44, wherein the marker corresponds to a transcribed polynucleotide or portion thereof, wherein the polynucleotide comprises the marker.

47. The method of claim 44, wherein the sample comprises cells obtained from the patient.

48. The method of claim 47, wherein the patient sample is a prostate tissue sample.

49. A method for assessing the aggressiveness or indolence of prostate cancer comprising comparing:
a) the level of expression of a marker in a sample, wherein at least one marker is selected from the markers of Tables 1-1 to 6, and
b) the normal level of expression of the marker in a control sample,
wherein a significant difference between the level of expression in the sample and the normal level is an indication that the cancer is aggressive or indolent.

50. The method of claim 49, wherein the marker corresponds to a secreted protein.

51. The method of claim 49, wherein marker corresponds to a transcribed polynucleotide or portion thereof, wherein the polynucleotide comprises the marker.

52. The method of claim 49, wherein the sample comprises cells obtained from the patient.

53. The method of claim 52, wherein the patient sample is a prostate tissue sample.

54. A system for identifying selected polynucleotide records that identify a prostate cancer cell, the system comprising:
a digital computer;
a database coupled to the computer;
a database coupled to the database server having data stored therein, the data comprising records of data comprising a polynucleotide corresponding to a marker from the markers in Tables 1-1 to 6; and
a code mechanism for applying queries based upon a desired selection criteria to the data file in the database to produce reports of polynucleotide records which match the desired selection criteria.

55. A method for detecting a prostate cancer cell, using a computer having a processor, memory, display, and input/output devices, the method comprising the steps of:
a) providing a sequence of a polynucleotide isolated from a sample suspected of containing a prostate cancer cell;
b) providing a database comprising records of data comprising a polynucleotide corresponding to a marker from the markers in Tables 1-1 to 6; and
c) using a code mechanism for applying queries based upon a desired selection criteria to the data file in the database to produce reports of polynucleotide records of step a) which provide a match of the desired selection criteria of the sequences in the database of step b), the presence of a match being a positive indication that the polynucleotide of step 1) has been isolated from a cell that is a prostate cancer cell.
